(19) United States
(12) Patent Application Publication
(10) Pub. No.: US 2025/0129070 A1
(43) Pub. Date: Apr. 24, 2025

(54) BIFUNCTIONAL ANDROGEN RECEPTOR COMPOUNDS

(71) Applicant: Kolm Therapeutics Inc., Woodbridge, CT (US)

(72) Inventors: Taavi NEKLESA, Orange, CT (US); Dominico VIGIL, Brookline, MA (US); Bruce A. LEFKER, Nahant, MA (US); Ralph P. ROBINSON, Gales Ferry, CT (US); Soumya RAY, Quincy, MA (US); Todd BOSANAC, Burlington, CT (US); Alexander W. HIRD, Belmont, MA (US)

(21) Appl. No.: 18/913,732

(22) Filed: Oct. 11, 2024

Related U.S. Application Data
(60) Provisional application No. 63/589,861, filed on Oct. 12, 2023.
(60) Provisional application No. 63/636,293, filed on Apr. 19, 2024.

(51) Int. Cl. C07D 471/04; A61K 9/00; A61K 9/08; A61K 9/20; A61K 9/48; A61K 47/55; C07D 401/14; C07D 403/12; C07D 403/14; C07D 417/14; C07D 487/04; C07D 519/00

(52) U.S. Cl. CPC C07D 471/04; A61K 9/0053; A61K 9/08; A61K 9/2013; A61K 9/2054; A61K 9/4825; A61K 47/55; C07D 401/14; C07D 403/12; C07D 403/14; C07D 417/14; C07D 487/04; C07D 519/00

(57) ABSTRACT

Described herein are heterobifunctional small molecules, methods of making, pharmaceutical compositions and medicaments comprising such heterobifunctional small molecules, and methods of using such heterobifunctional small molecules are described herein, in the treatment of diseases and conditions, such as cancer, autoimmune diseases, and inflammatory diseases.

### CROSS-REFERENCE TO RELATED APPLICATIONS

This application claims the benefit of U.S. Provisional Patent Application No. 63/589,861, filed Oct. 12, 2023, and U.S. Provisional Patent Application No. 63/636,293, filed Apr. 19, 2024, which are incorporated herein by reference in their entireties.

### FIELD OF THE INVENTION

Described herein are heterobifunctional small molecules, methods of making, pharmaceutical compositions and medicaments comprising such heterobifunctional small molecules, and methods of using such heterobifunctional small molecules are described herein, in the treatment of diseases and conditions, such as cancer, autoimmune diseases, and inflammatory diseases.

### BACKGROUND OF THE INVENTION

Cellular homeostasis, a key hallmark of living organisms, arises from interactions between biomolecules, such as protein (e.g., enzyme) and substrate interactions, within and outside the cell. Conventionally, the function of a particular protein in a particular disease state has been investigated and controlled through the use of monofunctional molecules (e.g., an inhibitor), which occupy the active site of the disease protein, thereby forming binary complexes that inhibit or downregulate activity of the disease proteins. Such monofunctional molecules have provided a conceptual pathway toward many FDA-approved drugs as a means for treating the disease state.

However, many monofunctional molecules lack specificity for the disease proteins in question leading to undesired toxicities or lack of efficacy, or the disease proteins adapts to the monofunctional molecules thereby leading to resistance. An alternative approach with monofunctional molecules is to target the coregulator or coactivator proteins of the disease proteins. However, such coregulator or coactivator proteins are typically present in every cell, healthy or diseased, and inhibiting their activity typically leads to narrower therapeutic indices.

Described herein are heterobifunctional small molecules that engage the androgen receptor (AR) and a disease-dependent protein, thereby forming ternary complexes only in the disease cells that express both proteins and leading to loss of function of the disease-dependent protein. The heterobifunctional small molecules described herein comprise at least one silent binder to the disease-dependent protein and at least one binder to AR. Such heterobifunctional small molecules offer a new means for treatment of diseases or conditions, with a wider therapeutic index and offer novel therapeutic targets vis a vis expansion of the disease proteins, such as AR.

### SUMMARY OF THE INVENTION

In some embodiments, disclosed herein is a heterobifunctional conditional inhibitor compound of Formula (I), or a pharmaceutically acceptable salt or solvate thereof:

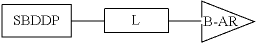

wherein:
SBDDP is a silent binder of a disease-dependent protein (DDP);
L is an optional linker; wherein L is covalently attached at a position of SBDDP that is solvent exposed when SBDDP binds to the DDP;
B-AR is a binder of the androgen receptor (AR), wherein B-AR comprises:
1) a head group that occupies the ligand-binding domain (LBD) of AR and is covalently attached to a core moiety; and
2) an optional tail moiety covalently attached to the core moiety; wherein the core comprises an optionally substituted cyclobutyl having the structure of Formula (C):

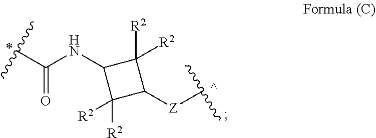

the head group is covalently attached to Z at position (\hat{ });
the optional tail moiety comprises a ring D that is covalently attached to the amide (*), wherein ring D is optionally substituted with s R³;
wherein: Z is —O— or —NR⁵—; and R² and R³ is described herein;
wherein L is covalently attached to the core moiety at position (*) if the optional tail moiety is absent, or L is covalently attached to the tail moiety if present;
wherein DDP and AR are both expressed in a cell of interest (COI), and wherein the relative abundance of the AR in the COI is greater than the relative abundance of the DDP in the COI; or the COI is a diseased cell, and the AR is overexpressed, overactive, or both overexpressed and overactive, or amplified in the diseased cell as compared to when the COI is a non-diseased cell.

In some embodiments, disclosed herein is a heterobifunctional conditional inhibitor compound of Formula (II), or a pharmaceutically acceptable salt or solvate thereof:

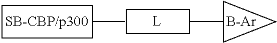

wherein:
SB-CBP/p300 is a silent binder of human CREB-binding protein (CBP) or human ETA-binding protein p300 (p300) (CBP/p300);
L is an optional linker; wherein L is covalently attached at a position of SB-CBP/p300 that is solvent exposed when SB binds to CBP/p300;
B-AR is a binder of the androgen receptor (AR), wherein the B-AR comprises:
1) ahead group that occupies the ligand-binding domain (LBD) of AR and is covalently attached to a core moiety; and
2) an optional tail moiety covalently attached to the core moiety; wherein the core comprises an optionally substituted cyclobutyl having the structure of Formula (C):

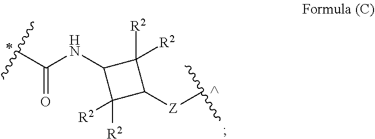

wherein: the head group is covalently attached to Z at position (\hat{ }); the optional tail moiety comprises a ring D that is covalently attached to the amide (*), wherein ring D is optionally substituted with s R³.
wherein: Z is —O— or —NR⁵—; and R² and R³ is described herein;
wherein L is covalently attached to the core moiety at position (*) if the optional tail moiety is absent, or L is covalently attached to the tail moiety if present.

In some embodiments, disclosed herein is a heterobifunctional conditional inhibitor compound of Formula (I), or a pharmaceutically acceptable salt or solvate thereof:

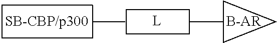

wherein:
SB-CBP/p300 is a silent binder of the bromodomain of human CREB-binding protein (CBP) or human ETA-binding protein p300 (p300) (CBP/p300), wherein the binder of CBP/p300 comprises:
an acetyl-lysine mimetic moiety that binds in the acetyl-lysine (KAc) binding site of the bromodomain of human CREB-binding protein (CBP) or human ETA-binding protein p300 (p300) (CBP/p300) and has the structure:

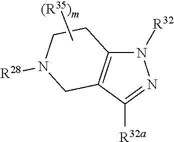

R²⁸ is —C(═O)CH₃, —C(═O)CH₂CH₃, —C(═O)NH₂, —C(═O)NH(CH₃), or —C(═O)NH(CH₂CH₃);
R³² is a moiety that occupies the BC Loop region of the bromodomain of CBP/p300;
R^{32a} is a moiety that occupies the lipophilic shelf (LPF) region of the bromodomain of CBP/p300; each R³⁵ is independently hydrogen, C₁-C₄alkyl, or C₁-C₄fluoroalkyl;
m is 0, 1, 2, 3, or 4;
L is an optional linker; wherein L is covalently attached to the R³² group, or at the position occupied by R³², or the R^{32a} group;
B-AR is a binder of the androgen receptor (AR), wherein the B-AR comprises:
   a head group that occupies the ligand-binding domain (LBD) of AR and is covalently attached to a core with a tail moiety covalently attached to the core moiety; wherein B-AR has the structure of Formula (D):

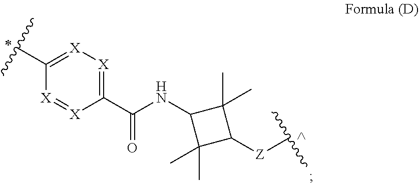

wherein head group is

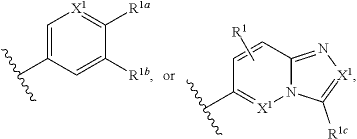

and the the head group is covalently attached to Z at position (\hat{ }), and L is attached at position *;
   Z is —O—, —NH— or —N(C₁-C₄alkyl)-; each X¹ is independently —CR¹— or —N—;
   R^{1a} is —CN, —NO₂, —C(═O)R⁵, —C(═O)OR⁵, or —C(═O)N(R⁵)₂;
   R^{1b} is hydrogen, halogen, substituted or unsubstituted C₁-C₄alkyl, C₁-C₄fluoroalkyl, —CN, —OH, or —OR⁴; R^{1c} is hydrogen, halogen, C₁-C₄alkyl, C₁-C₄fluoroalkyl, or —CN;
   each R¹ is independently hydrogen, halogen, substituted or unsubstituted C₁-C₄alkyl, C₁-C₄fluoroalkyl, —CN, —OH, or —OR⁴;
   each X is independently —CR³— or —N—;
   each R³ is independently hydrogen, halogen, substituted or unsubstituted C₁-C₄alkyl, C₁-C₄fluoroalkyl, —CN, —OH, —OR⁴, or —N(R⁵)₂.
   each R⁴ is independently substituted or unsubstituted C₁-C₄alkyl, or C₁-C₄fluoroalkyl;
   each R⁵ is independently hydrogen, substituted or unsubstituted C₁-C₄alkyl, C₁-C₄fluoroalkyl; and
   each s is 1, 2, or 3.

In some embodiments, disclosed herein is a stable ternary complex comprising:
a. one or more disease-dependent proteins (DDPs);
b. Androgen Receptor (AR); and
c. heterobifunctional conditional inhibitor compound as described herein;
   wherein DDP and AR are present in a cell of interest (COI) and the relative abundance of the AR in the COI is greater than the relative abundance of the DDP in the COI.

In some embodiments, the DDP is CREB-binding protein (CBP)/p300.

In some embodiments, disclosed herein is a stable ternary complex comprising:
d. CBP/p300;
e. Androgen receptor (AR); and
f. heterobifunctional conditional inhibitor compound as described herein; wherein CBP/p300 and DP are present in a cell of interest (COI) and the relative abundance of the DP in the COI is greater than the relative abundance of CBP/p300 in the COI.

In another aspect, described herein is a method of selectively inhibiting the activity of a disease-dependent protein (DDP) in a cell of interest (COI) of a mammal comprising administering a heterobifunctional compound as described herein, or a pharmaceutically acceptable salt or solvate thereof, wherein the COI expresses the androgen receptor (AR). In some embodiments, the heterobifunctional compound as described herein, or a pharmaceutically acceptable salt or solvate thereof, inhibits the activity of the DDP in the COI but does not inhibit the activity of the DDP in cells expressing the DDP and not expressing the AR. In some embodiments, the AR is overexpressed, overactive or both overexpressed and overactive in the COI. In some embodiments, the DDP is CREB-binding protein (CBP)/p300.

In another aspect, described herein is a method of treating cancer in a mammal comprising administering to the mammal a therapeutically effective amount of a compound as described herein, or a pharmaceutically acceptable salt or solvate thereof. In some embodiments, the cancer is a hormone dependent cancer. In some embodiments, the cancer is prostate cancer.

In another aspect, described herein is a method of treating an androgen receptor dependent or androgen receptor mediated disease or condition in mammal comprising administering to the mammal a therapeutically effective amount of a compound as described herein, or a pharmaceutically acceptable salt or solvate thereof. In some embodiments, the androgen receptor dependent or androgen receptor mediated disease or condition is selected from benign prostate hyperplasia, hirsutism, adenomas and neoplasms of the prostate, benign or malignant tumor cells containing the androgen receptor, prostate cancer, breast cancer, endometrial cancer, and uterine cancer.

Also described herein is a pharmaceutical composition comprising a heterobifunctional compound described herein, or a pharmaceutically acceptable salt, or solvate thereof, and at least one pharmaceutically acceptable excipient. In some embodiments, the pharmaceutical composition is formulated for administration to a mammal by oral administration, intravenous administration, or subcutaneous administration. In some embodiments, the pharmaceutical composition is formulated for administration to a mammal by oral administration. In some embodiments, the pharmaceutical composition is in the form of a tablet, a pill, a capsule, a liquid, or a suspension.

In one aspect, the heterobifunctional compounds described herein, or a pharmaceutically acceptable salt, or solvate thereof, are used in the treatment of diseases or conditions, such as cancer, autoimmune diseases, and inflammatory diseases. In some embodiments, the disease or condition is cancer. In some embodiments, the cancer comprises altered AR expression levels. In some embodiments, the cancer comprises altered androgen receptor expression levels. In some embodiments, altered DP expression levels comprises overexpressed AR, overactive AR, amplified AR, or combinations thereof.

In any of the embodiments disclosed herein, the mammal is a human. In some embodiments, compounds disclosed herein are orally administered to a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows the ternary complex formation assay for Compound 109 and Compound 106.

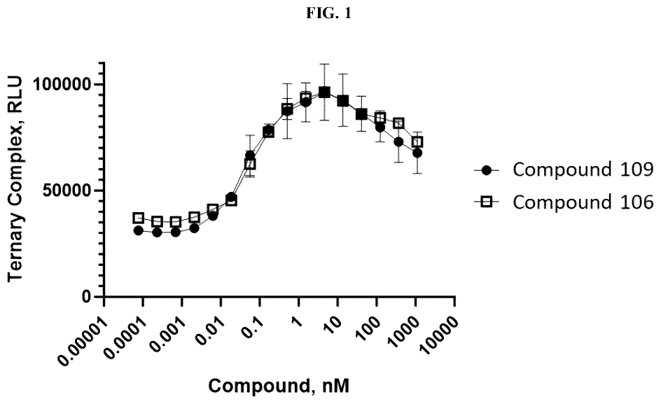

FIG. 2 shows the normalized bioluminescence resonance energy transfer (BRET) response as a function of compound concentration for Compound 109 and control GNE-781.

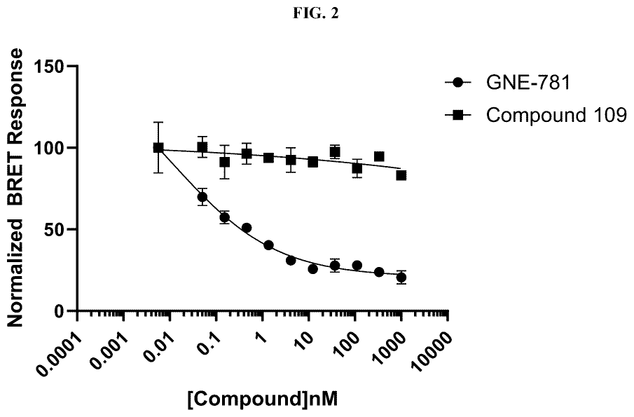

FIG. 3 shows the normalized bioluminescence resonance energy transfer (BRET) response as a function of compound concentration for GNE-781.

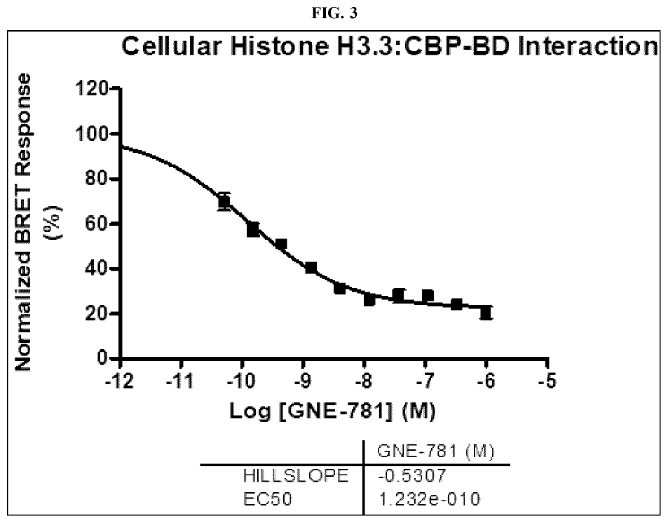

FIG. 4 shows the ternary complex formation assay for Compound 424 and Compound 360.

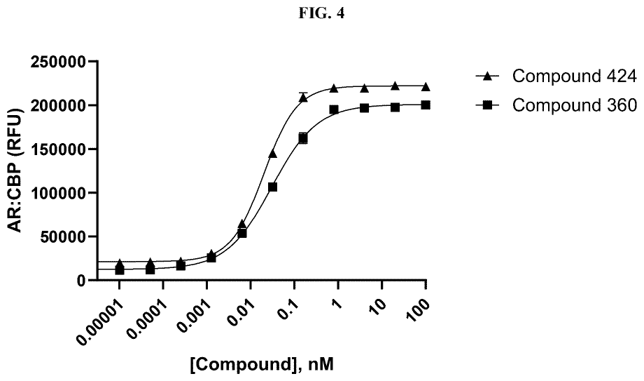

### DETAILED DESCRIPTION OF THE INVENTION

Normally, human cells grow and multiply through cell division to form new cells as the body needs them. Every cell in humans includes a collection of genes and proteins, many of which are required for survival and proliferation, and impairment of these genes and proteins leads to loss of fitness or cell death.

When cells grow old or become damaged, they die and new cells take their place. Sometimes this orderly process breaks down, and abnormal or damaged cells grow and multiply when they shouldn't. In the context of disease states, such as cancerous, autoimmune, or inflammatory states, certain proteins drive the disorderly growth and multiplication of abnormal or damaged cells.

Conventional monofunctional molecules (e.g., activators, inhibitors) form binary complexes with a target protein. In these binary complexes, the activators or inhibitors target a functional site, either orthosterically or allosterically, to modulate the target protein.

Many desired cellular changes cannot be accomplished through inhibition alone. Another class of molecules, i.e., bifunctional molecules, have been developed that operate by inducing proximity between the target proteins to form ternary complexes, which evokes a number of functions beyond inhibition.

Bifunctional molecules have primarily been utilized for their potential to simultaneously engage two macromolecular targets to form ternary complexes that in turn result in new and unique biological and cellular activities. The bifunctional molecules have seen promise in the applications of chemical induced dimerization, the inhibition of protein-protein interactions (PPIs), the degradation of target proteins, the simultaneous catalysis of two or more enzymatic processes, and the promotion or inhibition of protein aggregation. However, bifunctional molecules are not limited to the simultaneous engagement of targets. These molecules can be rationally designed from two functional chemical moieties for various dual functions such as the dual inhibition of synergistic proteins in diseases, targeted drug delivery, and activity-based profiling. Such applications have been proposed to replace combination therapies.

In ternary complexes, the bifunctional molecules can bind to various sites, including active or allosteric sites. While conventional inhibitors are occupancy driven, bifunctional molecules are often event driven. As a result, conventional inhibitors are stoichiometric, while bifunctional molecules can be sub-stoichiometric and catalytic. Furthermore, conventional inhibitors require strong binding affinities, whereas bifunctional molecules may exhibit low-to-moderate binding affinities to targeted proteins, as some ternary complexes rely on cooperativity. Compared with protein inhibitors, which can globally affect protein targets, these bifunctional molecules can be used to localize enzymatic activity to a given target. In addition, binary complexes have a saturation binding effect, where at high concentrations, the binding site is occupied. In contrast, ternary complexes can exhibit a hook effect, where high concentrations of the small molecule can saturate the two binding partners into individual binary complexes, resulting in loss of efficacy at a higher dose. Mathematical frameworks to describe the three-body equilibria have been developed to support experimental and theoretical findings of these ternary complexes (E. F. Douglass Jr., et al., A comprehensive mathematical model for three-body binding equilibria, J. Am. Chem. Soc., 135 (2013), pp. 6092-6099).

Cancer cells show extensive alterations in protein expression levels, which are drivers of their malignant transformation. Proteins with altered expression levels in cancer are involved in protein synthesis and degradation, signaling and metabolic pathways, DNA repair, apoptosis, and other cellular processes, whose alterations cause tumor development and progression.

A key component of successful drug development is the assessment of the therapeutic index (TI), the ratio of the dose or exposure of a drug required to elicit the desired therapeutic effect compared with the dose or exposure at which toxicity becomes limiting. While drugs with a high TI effectively kill cancer cells with manageable toxicities, drugs with a low TI cause significant side effects at or below efficacious doses. Cytotoxic chemotherapies, which typically target proliferating cells, generally have low TIs.

The modulation of disease protein levels is an effective anticancer target, which is achieved by targeting of up-regulated proteins in cancer, such as, but not limited to, androgen receptor, estrogen receptor, epidermal growth factor receptor 2 (HER2), and vascular endothelial growth factor (VEGF). The development of targeted therapeutics, typically monofunctional molecules, has provided alternative routes to achieving high TIs by either targeting cancer dysregulated genes with limited requirements for homeostasis in adults (e.g., ABL, KIT, TRK, ALK), or by developing mutation-biased inhibitors (e.g., KRAS^{G12C}).

However, targeting proteins that are required for survival and proliferation of cancer cells (i.e., disease-dependent proteins), such as cell cycle regulators, mitotic kinases, and epigenetic regulators, results in cellular loss of fitness or cell death of cancer cells, but the same proteins are also targeted in healthy cells. The result is a low TI drug that either fails approval by regulatory agencies or has limited use in treatment.

Described herein is a novel treatment modality for treating cancers, and other disease states. The novel heterobifunctional compounds described herein comprise at least two different monofunctional compounds, one that targets a disease protein and the other that targets a disease-dependent protein, with an optional linker connecting the two together. The targeting of the disease-dependent protein is achieved with a silent binder, such that the binding of the silent binder to the disease-dependent protein in the diseased cell (i.e., cancer cell) or healthy cells leads to minimal or no inhibition of the disease-dependent protein. When the binder of the disease protein binds to its target and the silent binder binds with its target, inhibition of disease-dependent protein is the result. The novel heterobifunctional compounds described herein are therapeutics with a high TI.

Protein pairs for the heterobifunctional compounds described herein can be chosen from resources such as, but not limited to: The Cancer Genome Atlas (TCGA), TCGA Pan-Cancer project, The Cancer Cell Line Encyclopedia (CCLE) consortium, the Genomics of Drug Sensitivity in Cancer (GDSC), Clinical Proteomic Tumour Analysis Consortium (CPTAC), Protein Interaction Network Analysis (PINA) platform, protein-protein interaction (PPI)network based on computational methods have been used to identify disease-specific genes, modules, and cancer-subtype subnetworks (Kann M G. Protein interactions and disease: computational approaches to uncover the etiology of diseases. Brief Bioinform. 2007; 8:333-46), Proteomics Database (PD), Dependency Map (DepMap), and Protein Abundance Database (PAXdb).

### Heterobifunctional Inhibitors

In some embodiments disclosed herein is a heterobifunctional inhibitor. In some embodiments, the heterobifunctional inhibitor is a heterobifunctional conditional inhibitor.

In some embodiments, the heterobifunctional conditional inhibitor compound is a compound of Formula (I):

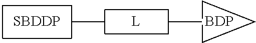

wherein:
SB is a silent binder of a disease-dependent protein (DDP);
L is an optional linker; and
BDP is a binder of a disease protein (DP), wherein the DP is the androgen receptor.

In some embodiments disclosed herein is a stable ternary complex comprising:
a. one or more disease-dependent proteins (DDPs);
b. a disease protein (DP), wherein the DP is the androgen receptor; and
c. heterobifunctional conditional inhibitor compound of Formula (I):

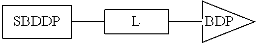

wherein:
SBDDP is a silent binder of a disease-dependent protein (DDP);
L is an optional linker; and
BDP is a binder of a disease protein (DP), wherein the DP is the androgen receptor; wherein DDP and DP are present in a cell of interest (COI) and the relative abundance of the DP in the COI is greater than the relative abundance of the DDP in the COI

In some embodiments disclosed herein is a stable ternary complex comprising:
a. CBP/p300;
b. a disease protein (DP), wherein the DP is the androgen receptor; and
c. heterobifunctional conditional inhibitor compound of Formula (II):

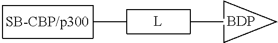

wherein:
SB-CBP/p300 is a silent binder of CBP/p300;
L is an optional linker; and
BDP is a binder of a disease protein (DP), wherein the DP is the androgen receptor;
wherein CBP/p300 and DP are present in a cell of interest (COI) and the relative abundance of the DP in the COI is greater than the relative abundance of CBP/p300 in the COI.

In some embodiments disclosed herein is a compound of Formula (III):

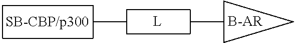

wherein:
SB-CBP/p300 is a silent binder of CBP/p300;
L is an optional linker; and
B-AR is a binder of the androgen receptor (AR).

In some embodiments, disclosed herein is a heterobifunctional compound of Formula (Ia), comprising:
a. a binder of human CREB-binding protein (CBP) or human E1A-binding protein p300 (p300) (CBP/p300);
b. a binder of a disease protein (DP); and
c. an optional linker covalently connecting a) to b);
wherein the optional linker is covalently attached at a position of a) that is solvent exposed when a) binds to CBP/p300.

In some embodiments, wherein SBDDP binds to the DDP and inhibits the activity of the DDP in the COI if the BDP simultaneously binds to the AR and the relative abundance of the DP in the COI is greater than the DDP in the COI.

In some embodiments, BDP is a non-silent binder or silent binder of a disease protein (DP). In some embodiments, BDP is a non-silent binder of a AR. In some embodiments, BDP is a silent binder of a disease protein AR.

In some embodiments, DDP and AR are both expressed in a cell of interest (COI). In some embodiments, CBP/p300 and DP are both expressed in a cell of interest (COI). In some embodiments, CBP/p300 and AR are both expressed in a cell of interest (COI).

In some embodiments, the relative abundance of the AR in the COI is greater than the relative abundance of the DDP in the COI. In some embodiments, the relative abundance of the DP in the COI is greater than the relative abundance of the DDP in the COI by a factor of at least about 2, at least about 5, at least about 10, at least about 50, at least about 100, or at least about 250. In some embodiments, the relative abundance of the DP in the COI is greater than the relative abundance of the DDP in the COI by a factor of at least about 100. In some embodiments, the relative abundance of the DP in the COI is greater than the relative abundance of CBP/p300 in the COI. In some embodiments, the relative abundance of the DP in the COI is greater than the relative abundance of CBP/p300 in the COI by a factor of at least about 2, at least about 5, at least about 10, at least about 50, at least about 100, or at least about 250. In some embodiments, the relative abundance of the DP in the COI is greater than the relative abundance of CBP/p300 in the COI by a factor of at least about 100. In some embodiments, the relative abundance of AR in the COI is greater than the relative abundance of the CBP/p300 in the COI. In some embodiments, the relative abundance of AR in the COI is greater than the relative abundance of CBP/p300 in the COI by a factor of at least about 2, at least about 5, at least about 10, at least about 50, at least about 100, or about least about 250. In some embodiments, the relative abundance of AR in the COI is greater than the relative abundance of CBP/p300 in the COI by a factor of at least about 100.

In some embodiments, the COI is a diseased cell, and the DP is overexpressed, overactive, or both overexpressed and overactive, or amplified in the diseased cell as compared to when the COI is a non-diseased cell. In some embodiments, the COI is a diseased cell, and the DP is overexpressed in the diseased cell as compared to when the COI is a non-diseased cell. In some embodiments, the COI is a diseased cell, and the DP is overactive in the diseased cell as compared to when the COI is a non-diseased cell. In some embodiments, the COI is a diseased cell, and the DP is overexpressed and overactive in the diseased cell as compared to when the COI is a non-diseased cell. In some embodiments, the COI is a diseased cell, and the DP is amplified in the diseased cell as compared to when the COI is a non-diseased cell. In some embodiments, COI is a diseased cell, and AR is overexpressed, overactive, or both overexpressed and overactive, or amplified in the diseased cell as compared to when the COI is a non-diseased cell.

In some embodiments, the activity of the DDP is reduced or inhibited by the compound of Formula (I), (II), or (III) when the DDP and DP are both expressed in the same COI and the relative abundance of the DP in the COI is greater than the relative abundance of the DDP in the COI. In some embodiments, the activity of the DDP is reduced by the compound of Formula (I), (II), or (III) when the DDP and DP are both expressed in the same COI and the relative abundance of the DP in the COI is greater than the relative abundance of the DDP in the COI. In some embodiments, the activity of the DDP is inhibited by the compound of Formula (I), (II), or (III) when the DDP and DP are both expressed in the same COI and the relative abundance of the DP in the COI is greater than the relative abundance of the DDP in the COI.

In some embodiments, the activity of the CBP/p300 is reduced or inhibited by the compound of Formula (Ia) when the CBP/p300 and DP are both expressed in the same COI and the relative abundance of the DP in the COI is greater than the relative abundance of the CBP/p300 in the COI.

In some embodiments, the activity of CBP/p300 is reduced or inhibited by the compound of Formula (III) when CBP/p300 and AR are both expressed in the same COI and the relative abundance of the AR in the COI is greater than the relative abundance of the CBP/p300 in the COI.

In some embodiments, the activity of the DDP is unaltered by the compound of Formula (I), (II), or (III) when the DDP and DP are not both expressed in the same COI and/or the relative abundance of the DP in the COI is not greater than the relative abundance of the DDP in the COI.

In some embodiments, the activity of the CBP/p300 is unaltered by the compound of Formula (Ia) when the CBP/p300 and DP are not both expressed in the same COI and/or the relative abundance of the DP in the COI is not greater than the relative abundance of the CBP/p300 in the COI.

In some embodiments, the activity of CBP/p300 is unaltered by the compound of Formula (III) when CBP/p300 and AR are not both expressed in the same COI and/or the relative abundance of the AR in the COI is not greater than the relative abundance of the CBP/p300 in the COI.

In some embodiments, the DP is the androgen receptor (AR) and the BDP is an AR binder. In some embodiments, the DP is the androgen receptor (AR) and the BDP is an AR antagonist or agonist. In some embodiments, the DP is the androgen receptor (AR) and the BDP is an AR antagonist comprising a N-(3-(3-chloro-4-cyanophenoxy)-2,2,4,4-tetramethylcyclobutyl)-aryl-carboxamide or a N-(3-(3-trifluroromethyl-4-cyanophenoxy)-2,2,4,4-tetramethylcyclobutyl)-aryl-carboxamide. In some embodiments, the DP is the androgen receptor (AR) and the BDP is an AR antagonist that is flutamide, hydroxylflutamide, nilutamide, bicalutamide, enzalutamide, apalutamide, ODM201, AZD3514, BMS641988, or N-[trans-3-(3-Chloro-4-cyanophenoxy)-2,2,4,4-tetramethylcyclobutyl]-1-(2-hydroxyethyl)-1H-pyrazole-4-carboxamide.

### Cell of Interest (COI)

In some embodiments, the COI is a diseased cell where inhibition of disease-dependent protein (DDP) is desirable. In some embodiments, the COI is a diseased cell. In some embodiments, the cell of interest is a cancer cell. In some embodiments, the cancer cell is associated with a carcinoma, sarcoma, leukemia, lymphoma, myeloma, or the central nervous system. In some embodiments, the cancer cell is associated with a carcinoma, for example, squamous cell carcinoma, adenocarcinoma, transitional cell carcinoma, or basal cell carcinoma. In some embodiments, the cancer cell is an epithelial cell, for example, a squamous cell, adenomatous cell, transitional cell, or basal cell. In some embodiments, the cancer cell is associated with a sarcoma, for example, bone sarcoma or soft tissue sarcoma. In some embodiments, the cancer cell is a bone cell, cartilage cell, or muscle cell. In some embodiments, the cancer cell is associated with a leukemia. In some embodiments, the cancer cell is a white blood cell. In some embodiments, the cancer cell is associated with a lymphoma or myeloma. In some embodiments, the cancer cell is a white blood cell or plasma cell. In some embodiments, the cancer cell is associated with the central nervous system, for example, the brain or spinal cord. In some embodiments, the cancer cell is a glial cell.

In some embodiments, the COI is a cell associated with head and neck cancer, laryngeal and hypopharyngeal cancer, nasal cavity and paranasal sinuses cancer, nasopharyngeal cancer, oral cavity (mouth) and oropharyngeal (throat) cancer, or salivary gland cancer. In some embodiments, the COI is a cell associated with anal cancer, bile duct cancer, colorectal cancer, esophagus cancer, gallbladder cancer, gastrointestinal neuroendocrine tumors, gastrointestinal stromal tumor, liver cancer pancreatic cancer, pancreatic neuroendocrine tumor, small intestine cancer, or stomach cancer. In some embodiments, the COI is a cell associated with associated with bladder cancer, kidney cancer, or Wilms tumor. In some embodiments, the COI is a cell associated with lung cancer, lung carcinoid tumor, or malignant mesothelioma. In some embodiments, the COI is a cell associated with breast cancer. In some embodiments, the COI is a cell associated with cervical cancer, endometrial cancer, ovarian cancer, penile cancer, prostate cancer, testicular cancer, uterine sarcoma, vaginal cancer, or vulvar cancer. In some embodiments, the COI is a cell associated with adrenal cancer, gastrointestinal neuroendocrine tumors, lung carcinoid tumor, pancreatic neuroendocrine tumor, pituitary tumors, or thyroid cancer. In some embodiments, the COI is a cell associated with skin cancer, basal and squamous cell skin cancer, Kaposi sarcoma, lymphoma of the skin, melanoma skin cancer, or Merkel cell skin cancer. In some embodiments, the COI is a cell associated with bone cancer, Ewing family of tumors, osteosarcoma, rhabdomyosarcoma, or soft tissue sarcoma. In some embodiments, the COI is a cell associated with eye cancer or retinoblastoma. In some embodiments, the COI is a cell associated with brain and spinal cord tumors or neuroblastoma. In some embodiments, the COI is a cell associated with leukemia, acute lymphocytic leukemia, acute myeloid leukemia, chronic lymphocytic leukemia, chronic myeloid leukemia, chronic myelomonocytic leukemia, lymphoma, non-Hodgkin lymphoma, Hodgkin-lymphoma, multiple myeloma, myelodysplastic syndromes, thymus cancer, or Waldenstrom macroglobulinemia.

In some embodiments, the COI is a cell associated with prostate cancer or breast cancer. In some embodiments, the COI is a cell associated with prostate cancer, luminal breast cancer, or luminal androgen receptor triple-negative breast cancer.

### Disease-Dependent Protein (DDP)

“Disease-dependent protein” or “DDP” refers to any one of the proteins expressed in cell of interest that is/are required for cell functioning and/or maintenance and/or survival in a particular disease. That is, the indicated disease is dependent on the disease-dependent protein to survive. In some embodiments, a cell of interest includes one type of DDP. In some embodiments, a cell of interest expresses more than one type of DDPs, and each type of DDPs performs functions distinct from any other type of DDPs in the cell of interest. In some embodiments, a cell of interest expresses more than one type of DDPs, and each type of DPPs performs functions substantially the same or overlapping with at least one other type of DDPs in the cell of interest.

In some embodiments, the disease-dependent protein (DDP) is AT-Hook containing transcription factor 1 (AHCTF1), Anaphase promoting complex subunit 1 (ANAPC1), ANAPC4, ANAPC5, Ataxia-telangiectasia mutated (ATM), Ataxia telangiectasia and Rad3-related protein (ATR), Aurora Kinase A (AurkA), Aurora Kinase B (AurkB), Bromodomain-containing protein 4 (BRD4), Bromodomain PHD finger transcription factor (BPTF), BUB1 mitotic checkpoint serine/threonine kinase B (BUB1B), CREB-binding protein (CBP)/p300, Cell division cycle 7-related protein kinase (CDC7), CDCl6, CDC23, CDC27, CDC45, Centromere protein W (CENPW), Chromatin assembly factor 1 subunit b (CHAFIB), Chromodomain helicase DNA binding protein 4 (CHD4), Checkpoint kinase 1 (CHK1), CHK2, Cleavage factor polyribonucleotide kinase subunit 1 (CLP1), CWC22, Cyclin-dependent kinase 1 (CDK1), CDK2, CDK4, CDK5, CDK6, CDK9, CDK11a, Cytoskeleton associated protein 5 (CKAP5), DDB1 and CUL4 associated factor 1 (DCAF1), DEAD-box helicase 1 (DBR1), DDX10, DDX41, DDX47, DDX54, DDX56, Dehydrodolichyl diphosphate synthase subunit (DHDDS), Deoxyhypusine synthase (DHPS), DEAH-box helicase 8 (DHX8), DONSON, DNA methyltransferase 1(DNMT1), Denticleless E3 ubiquitin protein ligase homolog (DTL), histone acetyltransferase P300 (EP300), E1A binding protein P400 (EP400), Extra spindle pole bodies like 1, separase (ESPL1)/separin, F-Box Protein 5 (FBXO5), Gem nuclear organelle associated protein 5 (GEMIN5), GINS2, GPN-Loop GTPase 3 (GPN3), HAUS augmin like complex subunit 1 (HAUS1), HAUS6, Host cell factor C1 (HCFC1), Histone deacetylase 1 (HDAC1), HDAC2, HDAC3, HEAT repeat containing 1 (HEATR1), Integrator complex subunit 11 (INTS11), Lysine acetyltransferase 8 (KAT8)/MYST1, Kinesin family member 11 (KIF11), KIF18A, KIF23, Mitotic arrest deficient 2 like 1 (MAD2A/MAD2L1), MAK16, Microtubule associate serine/threonine kinase like (MASTL), Midasin AAA ATPase 1 (MDN1), Mediator complex subunit 14 (MED14), MED11, MED28, Mitogen-activated protein kinase 1 (MEK1), MEK2, MIS18A, Methyl methanesulfonate-sensitivity protein 22-like (MMS22L), Myc, Mammalian target of rapamycin (MTOR), Neuronal precursor cell-expressed developmentally down-regulated protein 8 (NEDD8), NIP7, Nucleolar complex associated 4 homolog (NOC4L), Nucleolar protein 6 (NOL6), Notchless homolog 1 (NLE1), NUF2, Nucleoporin 205 (NUP205), NUS1, Opa interacting protein 5 (OIP5), Programmed cell death 11 (PDCD11), Phosphatidylinositol-3 kinase (PI3K), Polo-like kinase 1 (PLK1), DNA polymerase alpha 1, catalytic subunit (POLA1), DNA polymerase delta 1, catalytic subunit (POLD1), DNA polymerase epsilon, catalytic subunit (POLE), DNA polymerase epsilon, subunit 2 (POLE2), Peptidylprolyl isomerase domain and WD repeat containing 1 (PPWD1), Protein regulator of cytokinesis 1 (PRC1), DNA primase subunit 1 (PRIM1)/DNA PRIMASE, Protein arginine methyltransferase 5 (PRMT5), 20S proteasome subunits, RAD51, Rac GTPase activating protein 1 (RACGAP1), Ribonucleoside-diphosphate reductase subunit M1 (RRM1), Ribonucleic acid export 1 (RAE1), RNA polymerase I subunit B (POLR1B), RNA polymerase II subunit J (POLR2J), RNA polymerase II associated protein 1 (RPAP1), RNA guanylyltransferase and 5′-phosphatase (RNGTT), Splicing factor 3b subunit 1 (SF3B1), SWI/SNF related, matrix associated, actin dependent regulator of chromatin, subfamily A, member 2/4 (SMARCA2/4), Small nuclear RNA activating complex polypeptide 5 (SNAPC5), Structural maintenance of chromosomes 4 (SMC4), SMG1, SGO1, Steroid receptor coactivator 1 (SRC1), SRC2, SRC3, SLU7, SPC24, SPT6H, SPT5H, Symplekin scaffold protein (SYMPK), TIMELESS, Thioredoxin like 4a (TXNL4A), Tonsoku-like, DNA repair protein (TONSL), Topoisomerase II alpha (TOP2A), TPX2, Transformation/transcription domain associated protein (TRRAP), Trafficking protein particle complex subunit 8 (TRAPPC8), TSR2, Ubiquitously expressed transcript protein (UXT), URB1, UTP15, UTP20, Ubiquitin specific peptidase like 1 (USPL1), Vacuolar protein-sorting-associated protein 25 (VPS25), WD repeat-containing protein 3 (WDR3), WDR5, WDR12, WDR43, WDR46, WDR70, WD74, WEE1, WW domain binding protein 11 (WBP11), Xeroderma pigmentosum group A-binding protein 2 (XAB2), or Exportin 1 (XPO1).

In some embodiments, the disease-dependent protein (DDP) is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2, SMARCA4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3. In some embodiments, DDP is Aurora Kinase A (AurkA), Checkpoint kinase 1 (CHK1), CHK2, CDK4, CDK6, Myc, SMARCA2, SMARCA4, CREB-binding protein (CBP)/p300, WD repeat-containing protein 5 (WDR5), DDB1- and CUL4-associated factor 1 (DCAF1), phosphatidylinositol-3 kinase (PI3K), or WEE1. In some embodiments, DDP is Aurora Kinase A (AurkA), Checkpoint kinase 1 (CHK1), CHK2, CDK4, CDK6, Myc, SMARCA2, SMARCA4, CREB-binding protein (CBP)/p300, WD repeat-containing protein 5 (WDR5), DDB1- and CUL4-associated factor 1 (DCAF1), phosphatidylinositol-3 kinase (PI3K), or WEE1. In some embodiments, the DDP is CBP/p300 or SMARCA2/4. In some embodiments, DDP is CBP/p300. In some embodiments, the DDP is SMARCA 2/4.

In some embodiments, when the disease protein is the androgen receptor (AR), then the DDP is an AR coactivator or coregulator. Androgens, functioning through the AR, are essential for the normal development and maintenance of the prostate. Androgen-bound AR functions as a transcription factor to regulate genes involved in an array of physiological processes. The transcriptional activity of AR is affected by coregulators that influence a number of functional properties of AR, including ligand selectivity and DNA binding capacity. As the promoter of target genes, coregulators participate in DNA modification, either directly through modification of histones or indirectly by the recruitment of chromatin-modifying complexes, as well as functioning in the recruitment of the basal transcriptional machinery. Aberrant coregulator activity due to mutation or altered expression levels may be a contributing factor in the progression of diseases related to AR activity, such as prostate cancer.

The progression of prostate cancer is also sensitive to androgens. Surgical and/or pharmacological androgen ablation remain the predominant form of treatment for advanced prostate cancer. Androgen ablation therapy is often combined with treatment with nonsteroidal antiandrogens, such as hydroxyflutamide, to block residual adrenal androgen action. While 70-80% of patients initially respond to androgen ablation therapy, tumors ultimately become resistant and may, in fact, proliferate in response to antiandrogens. Because AR is generally expressed in prostate tumors and their metastases, aberrant regulation of AR activity by coregulators may contribute to prostate cancer progression or the acquired agonist effect of antiandrogens.

In addition, androgen-independent activation of the AR is a well-known phenomenon and can occur via several different mechanisms, including activation by interleukins. For example, interleukin-6 (IL-6) has been shown to activate AR-dependent gene expression in the absence of androgens. Activation of the AR and AR target gene expression by IL-6 requires p300 and its HAT activity. Similar to IL-6, interleukin-4 (IL-4) activates the AR, increases CBP/p300 protein expression, and enhances the interaction of CBP/p300 with the AR at the KLK3 promoter. Therefore, CBP/p300 appears to be crucial for AR transcriptional activity in both the presence and absence of androgens.

In some embodiments, AR coactivators or coactivator or coregulator is AKT1, AurkA, BAG family molecular chaperone regulator 1 (BAG1), beta-catenin, Breast cancer type 1 susceptibility protein (BRCA1), BRD4, C-jun, calmodulin 1, caveolin 1, CDK4/6, CDK9, Cytochrome C oxidase subunit 5b (COX5B), CBP/p300, CD1, CDK7, Dachshund family transcription factor 1 (DACH1), Death domain associated protein (Daxx), DCAF1, L-3,4-dihydroxyphenylalanine (L-DOPA), EF-hand calcium-binding domain-containing protein 6 (EFCAB6), Epidermal growth factor receptor (EGFR), Forkhead Box 01 (FOXO1), Glyceraldehyde-3-Phosphate Dehydrogenase (GAPDH), gelsolin, guanine nucleotide-binding protein subunit beta-2-like 1 (GNB2L1), Glycogen synthase kinase 3 beta (GSK3B), HDAC1, Heat shock protein 90 alpha family class a member 1 (HSP90AA1), HTATIP, MAGEA11, MED1, Myc, MYST2, Nuclear receptor coactivator 1 (NCOA1), NCOA2, NCOA3, NCOA4, NCOA6, Nuclear receptor corepressor 2 (NCOR2), Non-POU domain-containing octamer-binding protein (NONO), Proliferation-associated protein 2G4 (PA2G4), P21 (RAC1) Activated kinase 6 (PAK6), POZ/BTB and at hook containing zinc finger 1 (PATZ1), Protein Inhibitor Of Activated STAT 2 (PIAS2), Pre-MRNA Processing Factor 6 (PRPF6), Phosphatase And Tensin Homolog (PTEN), RAD9A, RAN binding protein 9 (RANBP9), Ring finger and CHY zinc finger domain containing 1 (RCHY1), Retinoblastoma protein, Ring finger protein 4 (RNF4), RNF14, Spliceosome associated factor 3, U4/U6 recycling protein (SART3), Sirtuin 1 (SIRT1), SMAD3, SMARCA2/4, small heterodimer partner, Src, Sex determining region Y (SRY), STAT3, Supervillin (SVIL), Testicular receptor 2, Testicular receptor 4, Transforming growth factor beta 1 induced transcript 1 (TGFBTIT), TATA element modulatory factor 1 (TMF1), Tripartite motif containing 68 (TRIM68), Ubiquitin conjugating enzyme E21 (UBE2I), Ubiquitously expressed prefoldin like chaperone (UXT), WEE1, or Zinc finger MIZ-type containing 1 (ZMIZ1).

### Silent Binder of a Disease-Dependent Protein (SBDDP)

“Silent-binder” refers to a compound (or fragment of a heterobifunctional compound) that binds to a target protein and does not substantially alter protein function. In some embodiments, a silent binder does not result in functional activation or inhibition of the target protein.

It is understood that some silent binders may, upon binding to a target protein, result in or induce some measurable or detectable effect on protein function. However, in such instances the measurable altered protein function is not detrimental to the ordinary functioning of the target protein at concentrations relevant for inducing inhibition of the target protein.

It is also understood that the silent binder to a target protein does not substantially alter protein function when it is incorporated into the heterobifunctional conditional inhibitor compounds disclosed herein unless the non-silent binder (NSB) component of the heterobifunctional conditional inhibitor compound also simultaneously binds to the disease protein (DP) in the same cell.

In some embodiments, a silent binder may be silent because it binds to a domain of a target protein that is not a relevant domain for the activity of the target protein. In other embodiments, a silent binder may be silent because it an allosteric binder of the target protein. In other embodiments, a silent binder may be silent because its incorporation into a heterobifunctional compound reduces the activity of the binder as compared to when it is not incorporated into a heterobifunctional compound. For example, a compound may be a modulator of a target protein when it is not a component of a heterobifunctional compound but becomes a silent binder of the target protein by virtue of its incorporation into a heterobifunctional compounds disclosed herein.

“Binder of a disease protein” or “BDP” refers to a compound (or fragment of a heterobifunctional compound) that binds to a target disease protein and: 1) does not substantially alter protein function; or 2) substantially alters protein function. In some embodiments, a binder of a disease protein when incorporated into the heterobifunctional compounds disclosed herein does not substantially alter protein function of the disease protein. In some embodiments, a binder of a disease protein when incorporated into the heterobifunctional compounds disclosed herein does not substantially alter protein function of the disease protein unless the silent binder component of the heterobifunctional conditional compound also simultaneously binds to a disease-dependent protein in the same cell.

“Non-silent binder” or “NSB” refers to a compound (or fragment of a heterobifunctional compound) that binds to a target disease protein and substantially alters protein function. NSBs include, but are not limited to, agonist, inverse agonist, antagonist, neutral antagonist, silent antagonist, competitive antagonist, irreversible antagonist, reversible antagonist, inhibitor, irreversible inhibitor, reversible inhibitor, allosteric modulator, negative allosteric modulator, and positive allosteric modulator.

Silent Antagonist is a drug that attenuates the effects of agonists or inverse agonists, producing a functional reduction in signal transduction. Affects only ligand-dependent receptor activation and displays no intrinsic activity itself. Also known as a neutral antagonist.

Agonist is a drug that binds to and activates a receptor. Can be full, partial or inverse. A full agonist has high efficacy, producing a full response while occupying a relatively low proportion of receptors. A partial agonist has lower efficacy than a full agonist. It produces sub-maximal activation even when occupying the total receptor population, therefore cannot produce the maximal response, irrespective of the concentration applied. An inverse agonist produces an effect opposite to that of an agonist yet binds to the same receptor binding-site as an agonist.

Allosteric Modulator is a drug that binds to a receptor at a site distinct from the active site. Induces a conformational change in the receptor, which alters the affinity of the receptor for the endogenous ligand. Positive allosteric modulators increase the affinity, whilst negative allosteric modulators decrease the affinity.

Antagonist is a drug that attenuates the effect of an agonist. Can be competitive or non-competitive, each of which can be reversible or irreversible. A competitive antagonist binds to the same site as the agonist but does not activate it, thus blocks the agonist's action. A non-competitive antagonist binds to an allosteric (non-agonist) site on the receptor to prevent activation of the receptor. A reversible antagonist binds non-covalently to the receptor, therefore can be “washed out”. An irreversible antagonist binds covalently to the receptor and cannot be displaced by either competing ligands or washing.

Efficacy describes the way that agonists vary in the response they produce when they occupy the same number of receptors. High efficacy agonists produce their maximal response while occupying a relatively low proportion of the total receptor population. Lower efficacy agonists do not activate receptors to the same degree and may not be able to produce the maximal response (see Agonist, Partial).

In some embodiments, SBDDP is a CBP/p300 binder. In some embodiments, SBDDP is a CBP/p300 bromodomain binder. In some embodiments, SBDDP is a CBP/p300 histone acetyltransferase (HAT) domain binder. In some embodiments, SBDDP is a CBP/p300 bromodomain binder comprising a benzimidazole, piperidine, benzodiazepinone, indole, oxazolidinedione, barbituric skeleton, thiobarbituric skeleton, or alkaloid. In some embodiments, SBDDP is a CBP/p300 binder as described in Zhang-Xu He, et al., European Journal of Medicinal Chemistry, Volume 209, 2021, 112861, which is incorporated by reference for such CBP/p300 binder. In some embodiments, SBDDP is a CBP/p300 binder as any one of compounds 1 to 75 as described in Zhang-Xu He, et al., European Journal of Medicinal Chemistry, Volume 209, 2021, 112861, which is incorporated by reference for such CBP/p300 binders.

In some embodiments, SBDDP has one of the following structures:

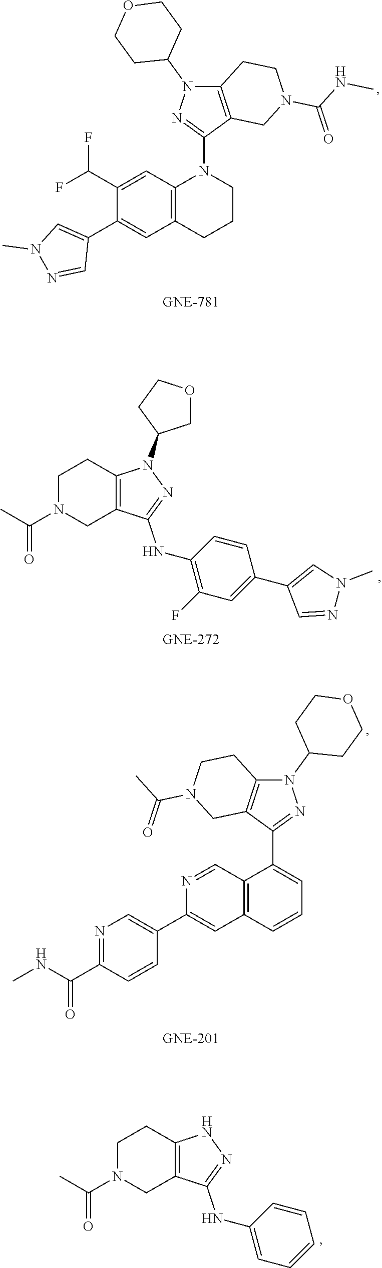

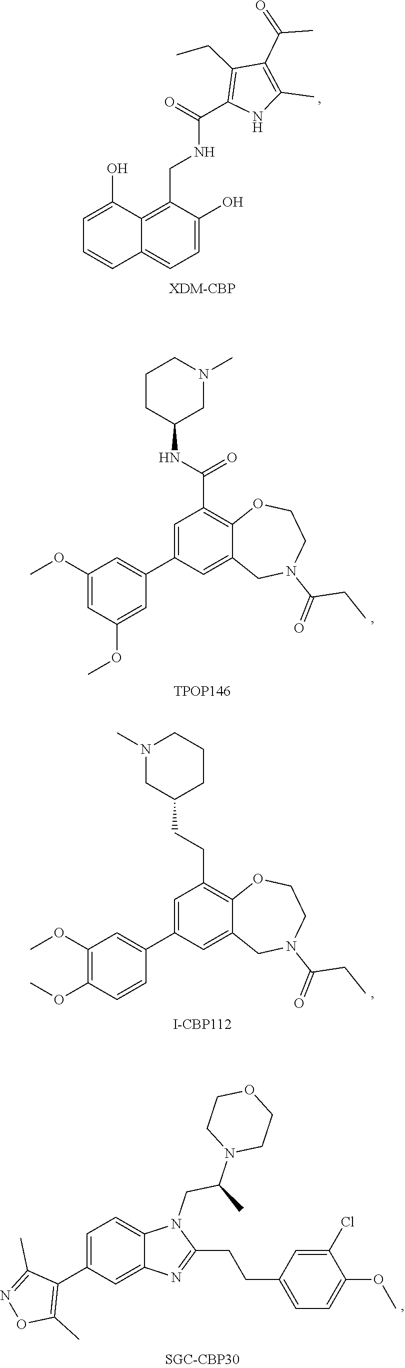

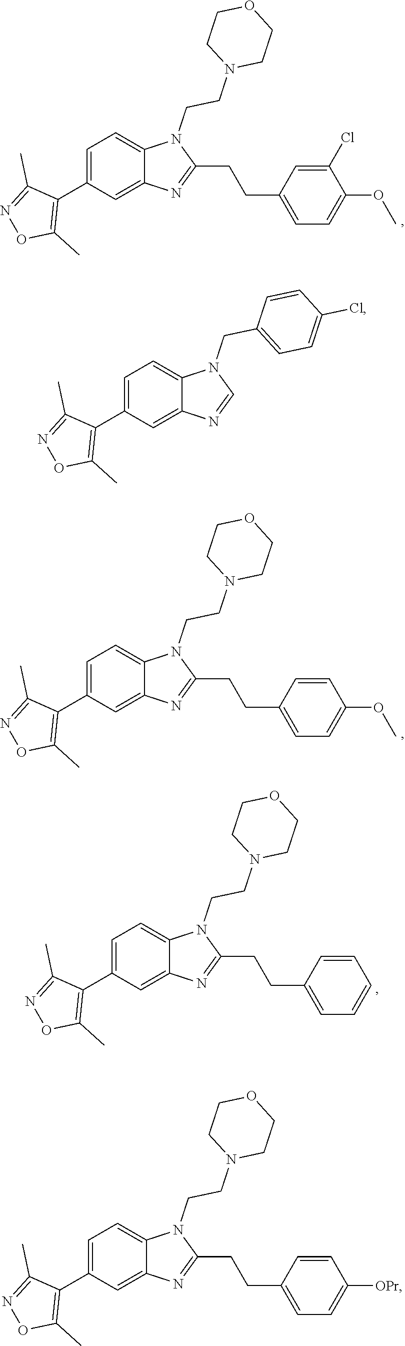

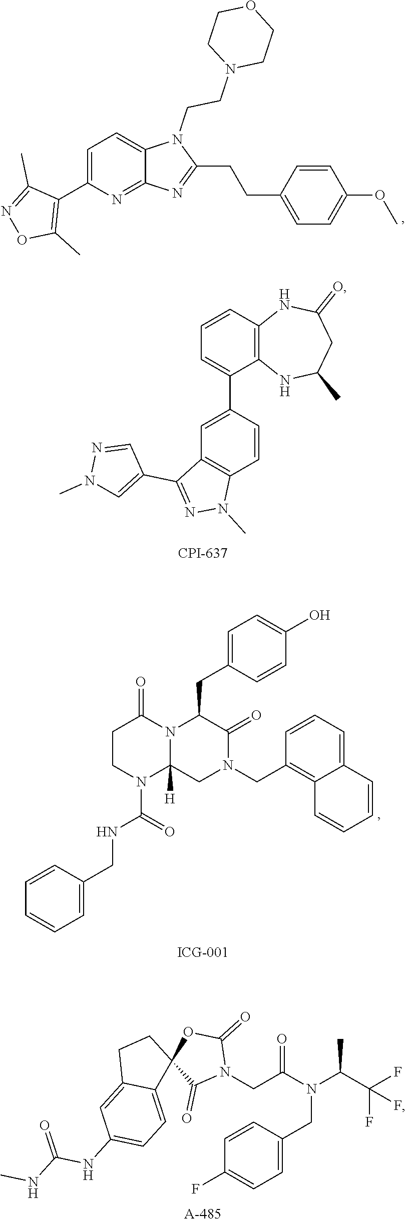

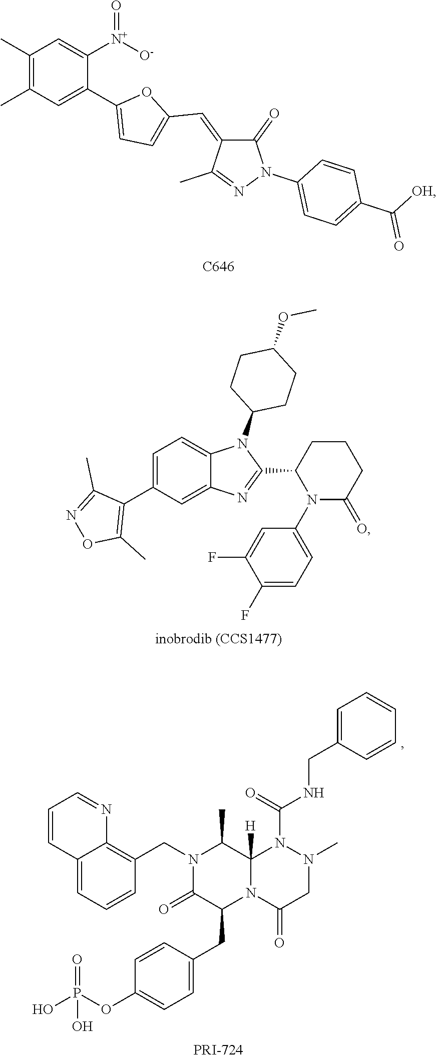 [SMILES: CC1=NN(c2ccc(C(=O)O)cc2)C(=O)/C1=C/c1ccc(-c2cc(C)c(C)cc2[N+](=O)[O-])o1 ; "C646" ; CO[C@H]1CC[C@H](n2c([C@@H]3CCCC(=O)N3c3ccc(F)c(F)c3)nc3cc(-c4c(C)noc4C)ccc32)CC1 ; C[C@H]1[C@H]2N(C(=O)CN(C)N2C(=O)NCc2ccccc2)[C@@H](Cc2ccc(OP(=O)(O)O)cc2)C(=O)N1Cc1cccc2cccnc12]

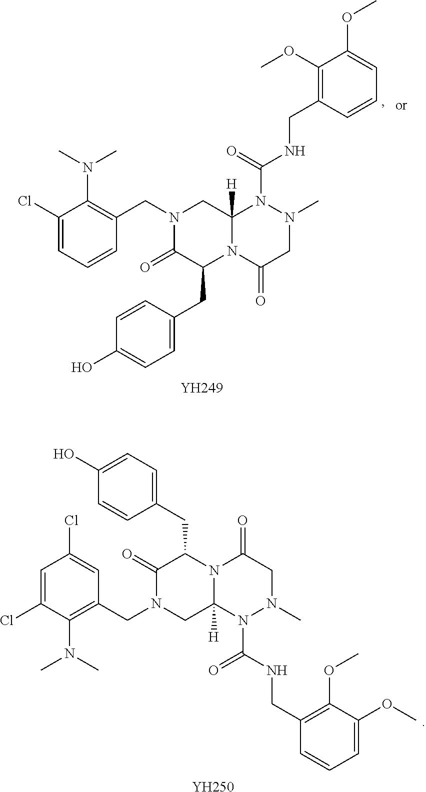 [SMILES: COc1cccc(CNC(=O)N2[C@H]3CN(Cc4cccc(Cl)c4N(C)C)C(=O)[C@H](Cc4ccc(O)cc4)N3C(=O)CN2C)c1OC ; "YH249" ; COc1cccc(CNC(=O)N2[C@H]3CN(Cc4cc(Cl)cc(Cl)c4N(C)C)C(=O)[C@H](Cc4ccc(O)cc4)N3C(=O)CN2C)c1OC ; "YH250"]

In some embodiments, SBDDP has one of the following structures:

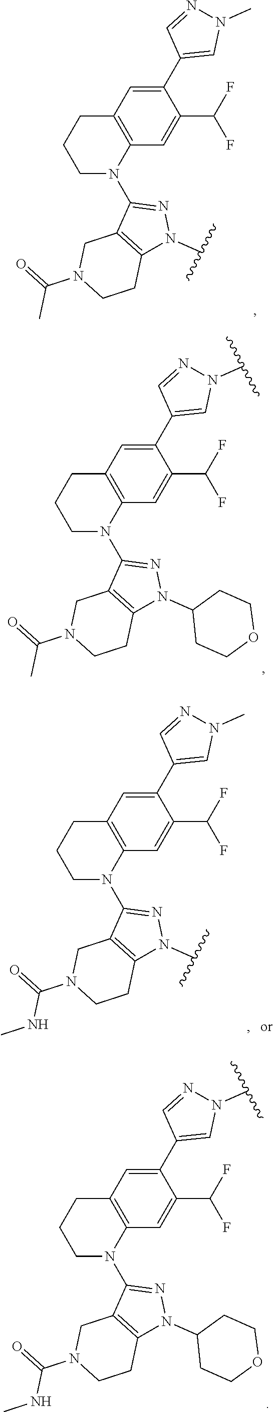

### Disease Protein (DP)

In some embodiments, the DP is a protein selectively expressed in a cancer or COI.

In some embodiments, the DP is a nuclear hormone receptor protein. In some embodiments, the DP is a nuclear hormone receptor, for example, androgen receptor (AR), estrogen receptor (ER), retinoic acid receptor (RAR), vitamin D receptor (VDR), glucocorticoid receptor (GR), mineralocorticoid receptor (MR), or progesterone receptor (PR). In some embodiments, the DP is AR or ER. In some embodiments, the DP is AR.

In some embodiments, the DP is AR.

### Binder of a Disease Protein (BDP)

In some embodiments, the BDP is a non-silent binder of a AR. In some embodiments, the BDP is a silent binder of a AR.

In some embodiments, the AR is a binder of the androgen receptor (AR), wherein B-AR comprises:
1) a head group that occupies the ligand-binding domain (LBD) of AR and is covalently attached to a core moiety, wherein the core comprises an optionally substituted cyclobutyl; and
2) an optional tail moiety covalently attached to the core moiety.

In some embodiments, the core comprises an optionally substituted cyclobutyl having the structure of Formula (C):

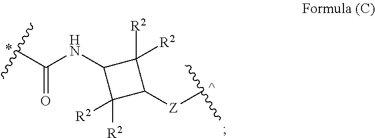

wherein: the head group is covalently attached to Z at position (\hat{ }); the optional tail moiety comprises a ring D that is covalently attached to the amide (*), each R² is —CH₃; wherein ring D is a 5-, 6-, 8-, 9- or 10-membered aryl or a 5-, 6-, 8-, 9- or 10-membered heteroaryl that is optionally substituted with s R³; Z is —O— or —NH— or —N(alkyl)-.

In some embodiments, the head moiety of the AR antagonist forms hydrogen bonds with the side chains of Gln 711 and Arg752 of the LBD of AR

In some embodiments, the head group of B-AR comprises 4-cyanophenyl; 3-fluoro-4-cyanophenyl; 3-chloro-4-cyanophenyl; 3-methoxy-4-cyanophenyl; 3-methyl-4-cyanophenyl; 3-trifluroromethyl-4-cyanophenyl; 3-trifluroromethoxy-4-cyanophenyl; 5-fluoro-6-cyanopyridin-3-yl; 5-chloro-6-cyanopyridin-3-yl; 5-methoxy-6-cyanopyridin-3-yl; 5-methyl-6-cyanopyridin-3-yl; 5-trifluroromethyl-6-cyanopyridin-3-yl; 5-trifluroromethoxy-6-cyanopyridin-3-yl; [1,2,4]triazolo[4,3-b]pyridazin-6-yl; or 3-(trifluoromethyl)-[1,2,4]triazolo[4,3-b]pyridazin-6-yl.

In some embodiments, the BDP is an AR antagonist. In some embodiments, the BDP is an AR antagonist comprising a N-(3-(3-chloro-4-cyanophenoxy)-2,2,4,4-tetramethylcyclobutyl)-aryl-carboxamide or a N-(3-(3-trifluroromethyl-4-cyanophenoxy)-2,2,4,4-tetramethylcyclobutyl)-aryl-carboxamide. In some embodiments, the BDP is an AR antagonist that is N-[trans-3-(3-Chloro-4-cyanophenoxy)-2,2,4,4-tetramethylcyclobutyl]-1-(2-hydroxyethyl)-1H-pyrazole-4-carboxamide.

In some embodiments, the head group and core of B-AR is:
N-(3-(3-fluoro-4-cyanophenoxy)-2,2,4,4-tetramethylcyclobutyl)acetamide-*;
N-(3-(3-chloro-4-cyanophenoxy)-2,2,4,4-tetramethylcyclobutyl)acetamide-*;
N-(3-(3-methoxy-4-cyanophenoxy)-2,2,4,4-tetramethylcyclobutyl)acetamide-*;
N-(3-(3-methyl-4-cyanophenoxy)-2,2,4,4-tetramethylcyclobutyl)acetamide-*;
N-(3-(3-trifluroromethyl-4-cyanophenoxy)-2,2,4,4-tetramethylcyclobutyl)acetamide-*;
N-(3-(3-trifluroromethoxy-4-cyanophenoxy)-2,2,4,4-tetramethylcyclobutyl)acetamide-*;
N-(3-((5-fluoro-6-cyanopyridin-3-yl)oxy)-2,2,4,4-tetramethylcyclobutyl)acetamide-*;
N-(3-((5-chloro-6-cyanopyridin-3-yl)oxy)-2,2,4,4-tetramethylcyclobutyl)acetamide-*;
N-(3-((5-methoxy-6-cyanopyridin-3-yl)oxy)-2,2,4,4-tetramethylcyclobutyl)acetamide-*;
N-(3-((5-methyl-6-cyanopyridin-3-yl)oxy)-2,2,4,4-tetramethylcyclobutyl)acetamide-*;
N-(3-((5-trifluroromethyl-6-cyanopyridin-3-yl)oxy)-2,2,4,4-tetramethylcyclobutyl)acetamide-*; or
N-(3-((5-trifluroromethoxy-6-cyanopyridin-3-yl)oxy)-2,2,4,4-tetramethylcyclobutyl)acetamide-*;
   wherein acetamide-* is

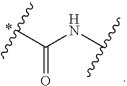 [SMILES: *NC(*)=O]

In some embodiments, the head group and core of B-AR is:
N-(3-(3-chloro-4-cyanophenoxy)-2,2,4,4-tetramethylcyclobutyl)acetamide-*;
N-(3-(3-methoxy-4-cyanophenoxy)-2,2,4,4-tetramethylcyclobutyl)acetamide-*;
N-(3-(3-methyl-4-cyanophenoxy)-2,2,4,4-tetramethylcyclobutyl)acetamide-*; or
N-(3-(3-trifluroromethyl-4-cyanophenoxy)-2,2,4,4-tetramethylcyclobutyl)acetamide-*;
wherein acetamide-* is

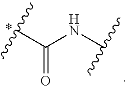 [SMILES: *NC(*)=O]

In some embodiments, B-AR further comprises a tail moiety that is covalently attached to position (*), wherein the tail moiety is a ring D that is phenyl, pyrimidinyl, pyrazinyl, pyridazinyl, triazinyl, triazolyl, pyrrolyl, imidazolyl, pyrazolyl, pyridyl, indolyl, isoindolyl, indolizinyl, benzimidazolyl, napthyl, quinolyl, isoquinolyl, quinoxalinyl, quinazolinyl, indazolyl, or benzotriazolyl; wherein ring D is optionally substituted with s R³; s is 1, 2, or 3; each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂, or —C(═O)NH(CH₃).

### Representative Approaches to Designing Heterobifunctional Compounds

The heterobifunctional compounds described herein include three component parts: the ligand that binds the target protein (i.e., the disease protein), the ligand that binds to the disease-dependent protein (DDP), and the linker. Crystal structures of many of these proteins are known and the binding interactions of each component within its respective binding site can be visualized with suitable modeling software. In addition, computer modeling can also be performed to determine the placement of the two proteins relative to one another.

The Protein Data Bank (PDB) is a database for the three-dimensional structural data of large biological molecules, such as proteins and nucleic acids (Nucleic Acids Res. 2019 Jan. 8; 47(D1):D520-D528. doi: 10.1093/nar/gky949). The data is submitted by biologists and biochemists from around the world, are freely accessible on the Internet via the websites of its member organizations (e.g. PDBe—www.pdbe.org, PDBj—www.pdbj.org, RCSB—www.rcsb.org/pdb, and BMRB—www.bmrb.wisc.edu). The PDB is overseen by an organization called the Worldwide Protein Data Bank—wwPDB—www.wwpdb.org.

In some embodiments, searching on the PDB database and manipulating of protein 3D models is performed with PyMOL. PyMOL is a user-sponsored molecular visualization system on an open-source foundation, maintained and distributed by Schrödinger. Other PDB database search engines and molecular visualization system are contemplated.

Model of ligand docking in candidate proteins, and modelling of ternary complexes can be built in the Rosetta software suite (Leaver-Fay A, et al., ROSETTA3: an object-oriented software suite for the simulation and design of macromolecules. Methods Enzymol. 2011; 487:545-74). Novel silent binders can be identified for DDPs using methods known in the art, such as fragment-based lead discovery (FBLD) (Kirsch P.; et al., Concepts and Core Principles of Fragment-Based Drug Design. Molecules 2019, 24 (23), 4309; Lamoree B.; et al. Current perspectives in fragment-based lead discovery (FBLD). Essays Biochem. 2017, 61 (5), 453-464; Erlanson D. A.; et al. Fragment-Based Drug Discovery. J. Med. Chem. 2004, 47 (14), 3463-3482; Shuker S. B.; et al. Discovering high-affinity ligands for proteins: SAR by NMR. Science 1996, 274 (5292), 1531-1534), DNA-Encoded Library (DEL) approaches and traditional high-throughput screening (HTS) methods.

Screening libraries used in FBLD are composed of small molecules called fragments that are broadly compliant with what is now widely recognized as the rule-of-three (Ro3) (Congreve M.; et al., ‘Rule of Three’ for fragment-based lead discovery?. Drug Discovery Today 2003, 8 (19), 876-877; Jhoti H.; et al., The ‘rule of three’ for fragment-based drug discovery: where are we now?. Nat. Rev. Drug Discovery 2013, 12 (8), 644-644). The small size and simplicity of fragments enables a minimalist approach to drug discovery, whereby a vast chemical space can be efficiently covered using libraries of only a few hundred molecules. This makes fragment screening appealing to industry and academic investigators alike and represents a very accessible, cost-effective, and sustainable approach to hit discovery. Furthermore, unlike conventional libraries used for HTS, fragment libraries are not biased toward previously explored targets. This makes FBLD an indispensable method for assessing the ligandability of novel proteins' binding sites, including PPI sites.

In some embodiments, the small size of fragments, however, results in them having weak binding affinities to proteins. For this reason, fragments tend to be screened at high concentrations using biophysical techniques such as nuclear magnetic resonance (NMR), surface plasmon resonance (SPR), differential scanning fluorimetry (DSF; also known as thermal shift assay (TSA)), and X-ray crystallography. Weak binders can be skillfully optimized into bespoke potent ligands for proteins via fragment growing, merging, and hybridizing methodologies. However, the successful pursuit of such approaches is often contingent on the determination of the binding mode of the fragment hits to the target protein, typically using X-ray crystallographic methods and/or ¹⁵N 2D-NMR experiments and/or cryogenic electron microscopy (cryo-EM) (Saur M.; et al., Fragment-based drug discovery using cryo-EM. Drug Discovery Today 2020, 25 (3), 485-490).

DEL is achieved through combinatorial chemistry and DNA-encoding techniques. With library modularity, DELs can be built in a time-saving and labor-saving way. This technology can construct and screen unprecedented scale combinatorial compound libraries (hundreds of billions scale) and discover numerous high-affinity ligands with high efficiency and low cost through protein target affinity screening and high-throughput sequencing and decoding (Buller et al., (2010). Drug discovery with DNA-encoded chemical libraries. Bioconjug. Chem. 21 (9), 1571-15802010; Kalliokoski T. (2015). Price-focused analysis of commercially available building blocks for combinatorial library synthesis. ACS Comb. Sci. 17 (10), 600-607; Goodnow et al., (2017). DNA-Encoded chemistry: Enabling the deeper sampling of chemical space. Nat. Rev. Drug Discov. 16 (2), 131-147). DEL can be used to create compound libraries with higher molecular weight. Empirically, such DEL libraries appear well suited for discovering ligands for protein-protein interaction (PPI) targets, which are increasingly needed for hits.

Linkers can be designed and assessed as well. Capturing the breadth of viable linker conformations is much akin to modeling the flexibility of drug-like molecules when docking them to proteins. For this purpose, the OMEGA software (Hawkins P C, et al., Comparison of shape-matching and docking as virtual screening tools. J Med Chem. 2007; 50:74-82; Hawkins P C, Nicholls A. Conformer generation with OMEGA: learning from the data set and the analysis of failures. J Chem Inf Model. 2012; 52:2919-36).

### Compounds

In some embodiments, the heterobifunctional conditional inhibitor compound is a compound of Formula (I):

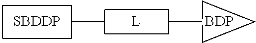

wherein:
SB is a silent binder of a disease-dependent protein (DDP);
L is an optional linker; and
BDP is a binder of a disease protein (DP).

In some embodiments, the BDP is an AR antagonist. In some embodiments, the BDP is an AR antagonist comprising a N-(3-(3-chloro-4-cyanophenoxy)-2,2,4,4-tetramethylcyclobutyl)-aryl-carboxamide or a N-(3-(3-trifluroromethyl-4-cyanophenoxy)-2,2,4,4-tetramethylcyclobutyl)-aryl-carboxamide. In some embodiments, the BDP is an AR antagonist that is N-[trans-3-(3-Chloro-4-cyanophenoxy)-2,2,4,4-tetramethylcyclobutyl]-1-(2-hydroxyethyl)-1H-pyrazole-4-carboxamide.

In some embodiments disclosed herein are heterobifunctional conditional inhibitor compounds of Formula (III):

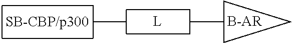

wherein:
SB-CBP/p300 is a silent binder of CBP/p300;
L is an optional linker; and
B-AR is a binder of the androgen receptor (AR).

In some embodiments, disclosed herein is a heterobifunctional compound of Formula (Ia), comprising:
a. a binder of human CREB-binding protein (CBP) or human E1A-binding protein p300 (p300) (CBP/p300);
b. a binder of a disease protein (DP); and
c. an optional linker covalently connecting a) to b);
   wherein the optional linker is covalently attached at a position of a) that is solvent exposed when a) binds to CBP/p300.

In some embodiments, the binder of CBP/p300 binds in the acetyl-lysine (Kac) binding site of the bromodomain CBP/p300. In some embodiments, the binder of CBP/p300 comprises an acetyl-lysine mimetic moiety that binds in the acetyl-lysine (KAc) binding site of the bromodomain of human CREB-binding protein (CBP) or human E1A-binding protein p300 (p300) (CBP/p300).

In some embodiments, the optional linker is covalently attached at a position of a) that is solvent exposed when a) binds the KAc binding site of the bromodomain of CBP/p300.

In some embodiments, the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300.

In some embodiments, a) further comprises a moiety that interacts with Arg1173 in the bromodomain of CBP or Asn1137 in the bromodomain of p300.

In some embodiments, wherein a) further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2).

In some embodiments, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

In some embodiments, the optional linker of c) is covalently attached to a) on: the acetyl-lysine mimetic moiety; or the moiety that occupies the lipophilic shelf (LPF) region of the bromodomain of CBP/p300, if present; or the moiety that occupies the BC Loop region of the bromodomain of CBP/p300, if present; wherein the optional linker of c) is covalently attached to a) at a position that does not interfere with the binding of the acetyl-lysine mimetic moiety in the acetylated lysine (KAc) binding site of CBP/p300.

In some embodiments, the moiety comprising an acetyl-lysine mimetic comprises a moiety selected from:

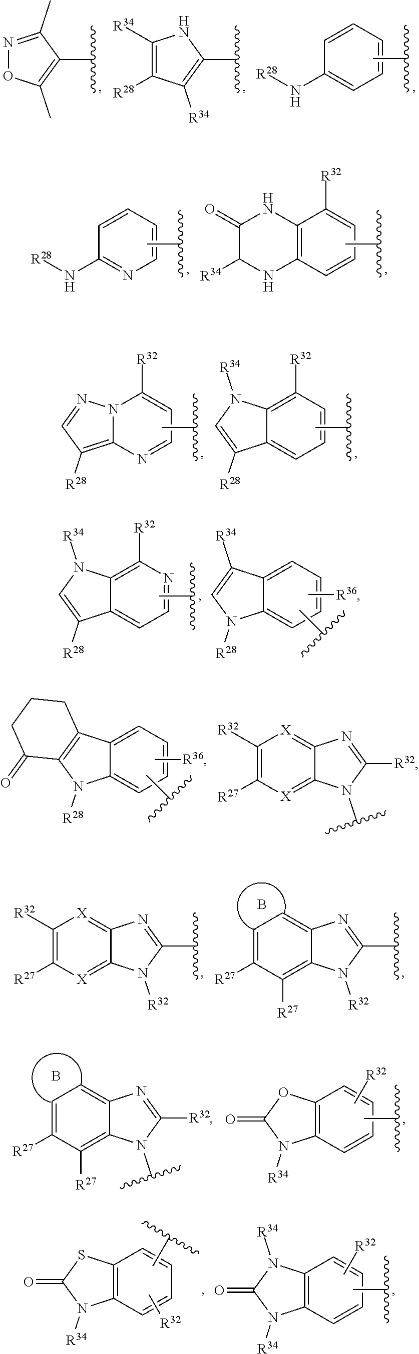

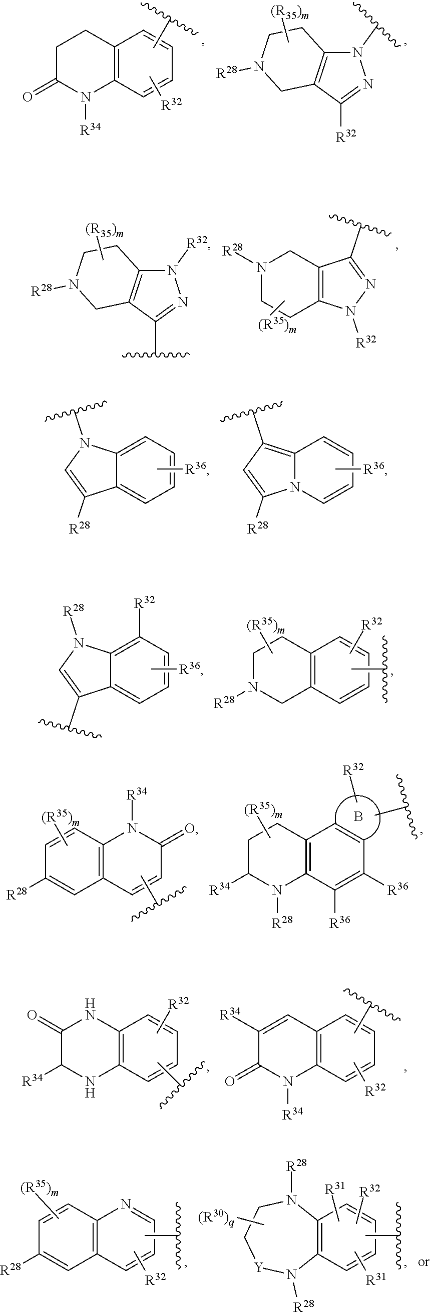

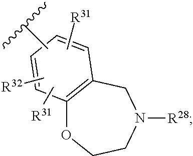

each R³² is independently an optional moiety that occupies the lipophilic shelf (LPF) region of the bromodomain of CBP/p300;
or each R³² is independently an optional moiety that occupies the BC Loop region of the bromodomain of CBP/p300;

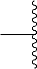

is the point of attachment to the optional linker that covalently connects a) to b);
or

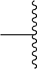

is R³² and the optional linker that covalently connects a) to b) is attached to R³²;
each R²⁸ is independently hydrogen, substituted or unsubstituted C₁-C₆alkyl, substituted or unsubstituted C₁-C₆fluoroalkyl, —C(═O)R^{b}, or —C(═O)N(R^{b})₂;
each R³⁴ is independently hydrogen or substituted or unsubstituted C₁-C₆alkyl;
each R³⁵ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl, —CN, —OH, —OR^{a}, or —N(R^{b})₂;
m is 0, 1, 2, 3, or 4;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O); each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
each R³⁶ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
Ring B is a fused substituted or unsubstituted 5 or 6 membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl;
or R³² is

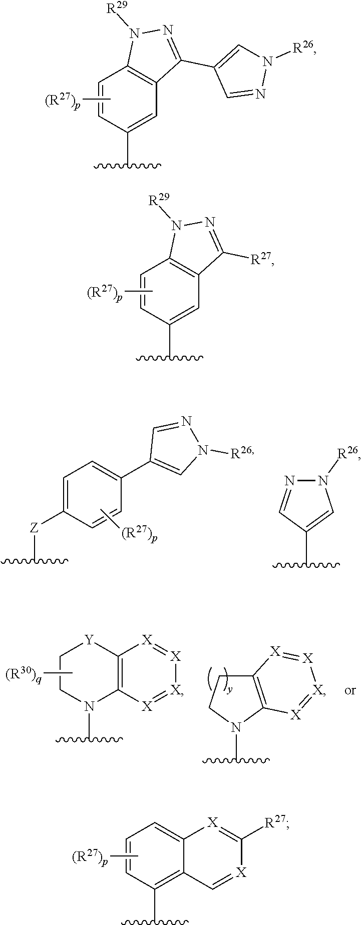

y is 1 or 2;
Z is —NR^{c}—, —O—, or —S—;
   R^{c} is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each X is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Y is —C(R³⁰)₂- or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
or R³² is -L-C;
L is substituted or unsubstituted C₁-C₆alkyl or substituted or unsubstituted C₁-C₆heteroalkyl;
C is substituted or unsubstituted C₃-C₈ cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, the moiety that occupies the BC Loop region of the bromodomain of CBP/p300 is R³², wherein:
R³² is

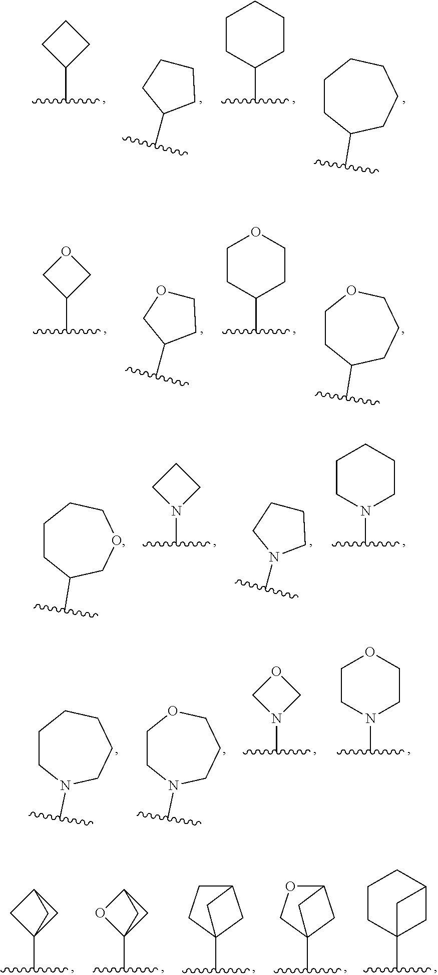

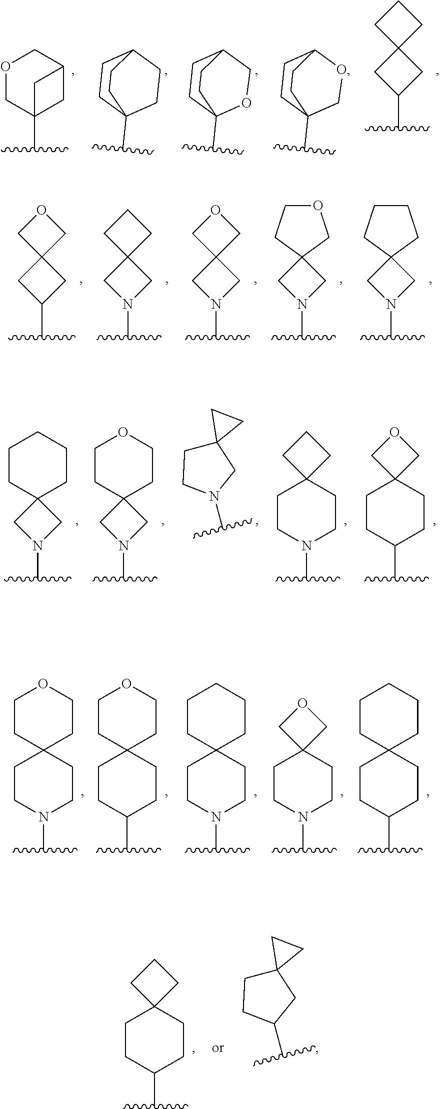

each of which is substituted or unsubstituted.

In some embodiments, the moiety comprising an acetyl-lysine mimetic comprises

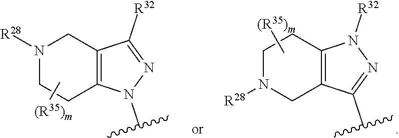

In some embodiments, the moiety comprising an acetyl-lysine mimetic comprises

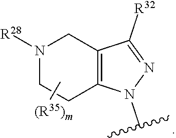

In some embodiments, the moiety comprising an acetyl-lysine mimetic comprises

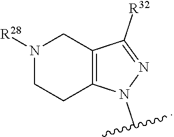

In some embodiments, the moiety comprising an acetyl-lysine mimetic comprises

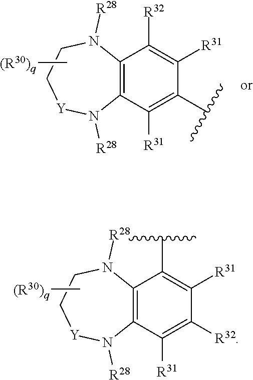

In some embodiments, the moiety comprising an acetyl-lysine mimetic comprises

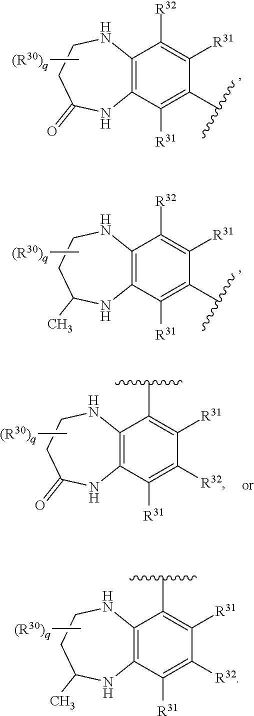

In some embodiments, the moiety comprising an acetyl-lysine mimetic comprises

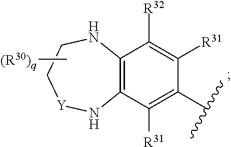

wherein:
each R²⁸ is independently hydrogen or substituted or unsubstituted C₁-C₆alkyl;
R³² is

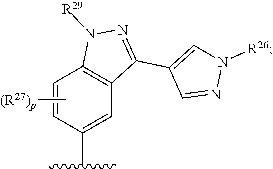

and
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂.

In some embodiments, the moiety comprising an acetyl-lysine mimetic comprises

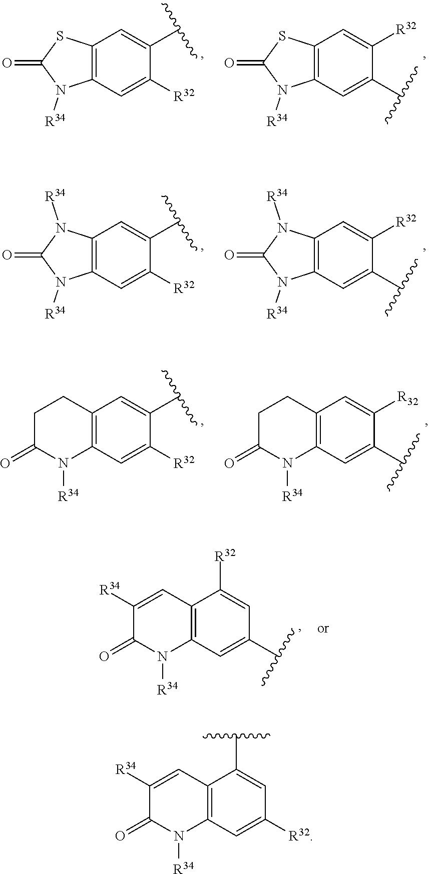

In some embodiments, the moiety comprising an acetyl-lysine mimetic comprises

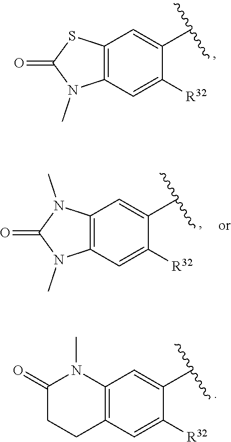

In some embodiments, the moiety comprising an acetyl-lysine mimetic comprises

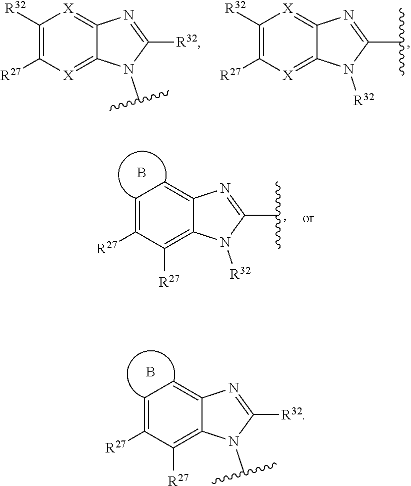

embodiments, the moiety comprising an acetyl-lysine mimetic comprises the moiety comprising an acetyl-lysine mimetic comprises

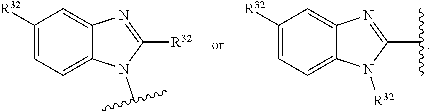

In some embodiments,

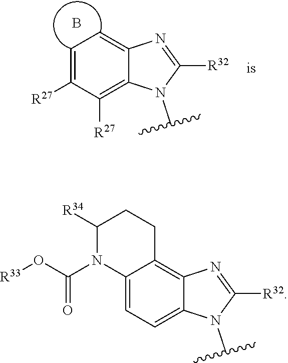

In some embodiments, the moiety comprising an acetyl-lysine mimetic comprises

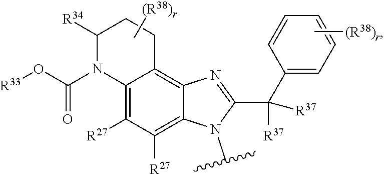

wherein:
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
R³³ is hydrogen, substituted or unsubstituted C₁-C₆alkyl, substituted or unsubstituted C₁-C₆fluoroalkyl, or substituted or unsubstituted C₁-C₆heteroalkyl;
R³⁴ is hydrogen or substituted or unsubstituted C₁-C₆alkyl;
each R³⁸ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, —CN, —NO₂, —OH, —OR^{a}, —OC(═O)R^{a}, —OC(═O)N(R^{b})₂, —OC(═O)OR^{a}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
r is 0, 1, 2, 3, or 4;

In some embodiments, R³² is

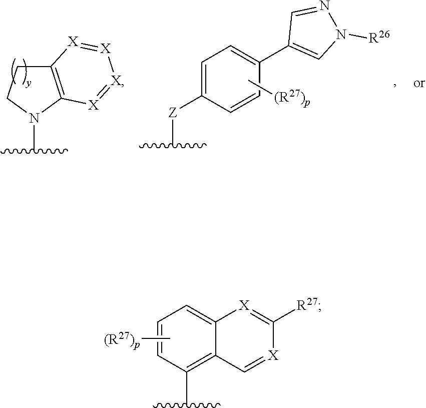

each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and each R^{a} is independently substituted or unsubstituted C₁-C₆alkyl.

In some embodiments, R³² is

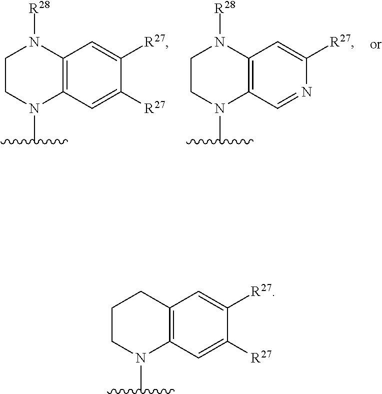

In some embodiments, each R²⁷ is independently hydrogen, —CH₃, —CH₂CH₃, —F, —CHF₂, —CF₃, —CN, —OH, —OCH₃, Cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

In some embodiments, R³² is

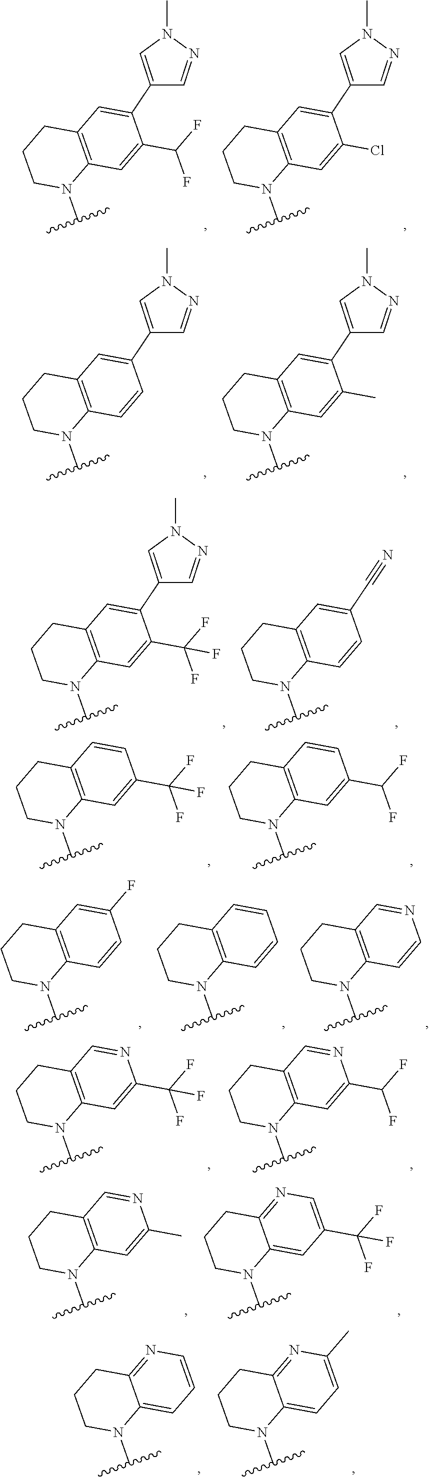

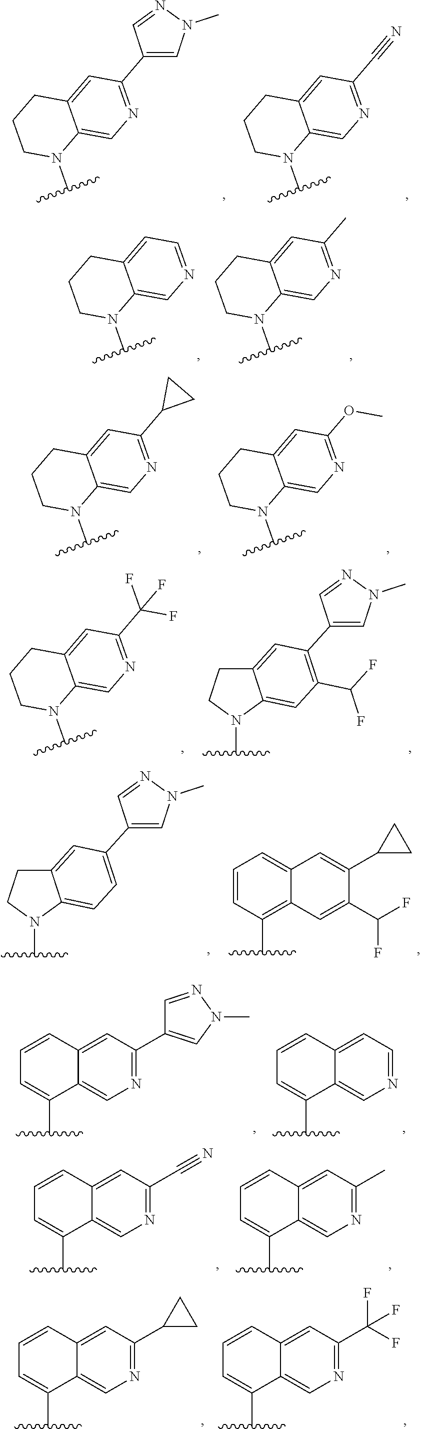

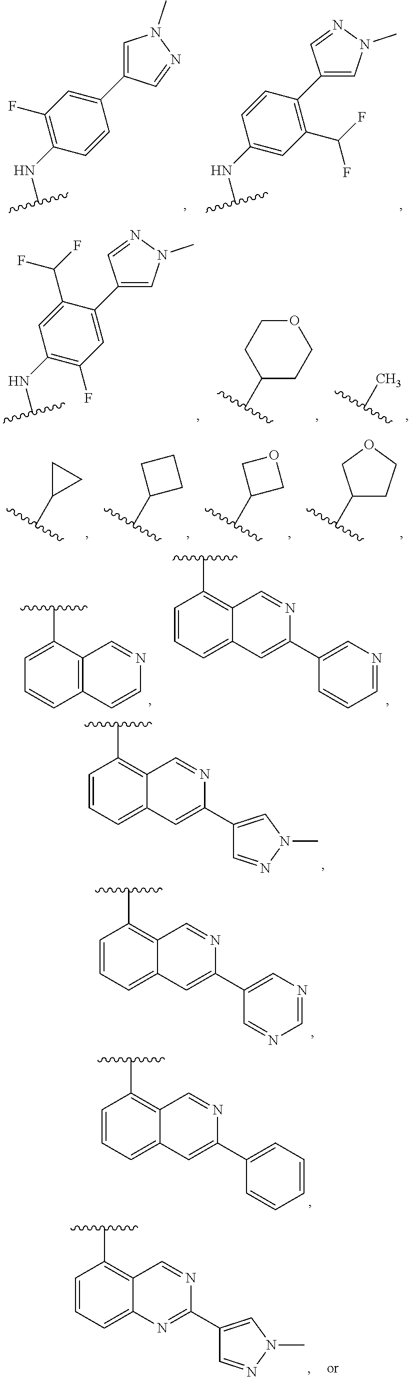

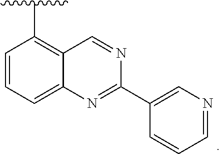 [SMILES: *c1cccc2nc(-c3cccnc3)ncc12]

In some embodiments, R³² is

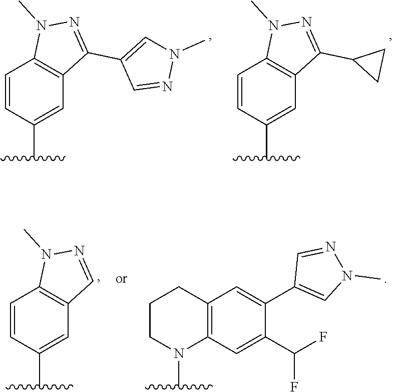 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

In some embodiments, R³² is

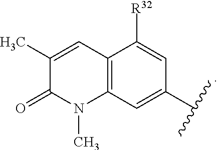

In some embodiments, R³² is

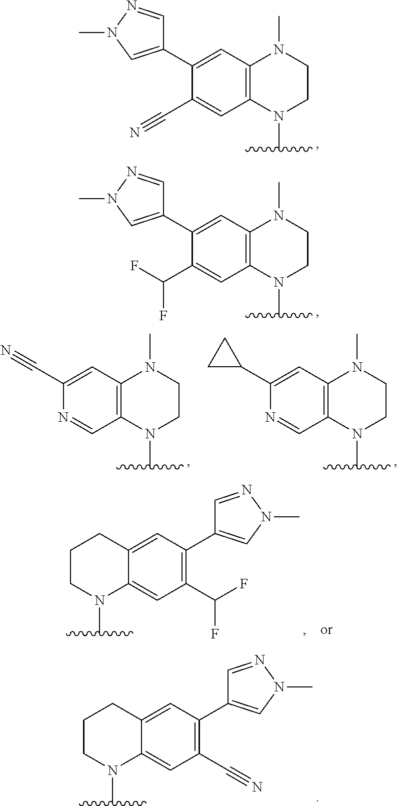

In some embodiments, R³² is

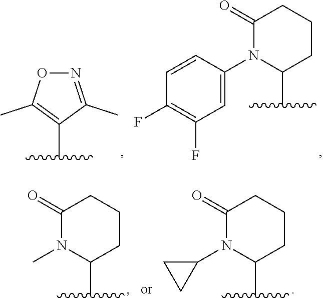

In some embodiments, R³² is

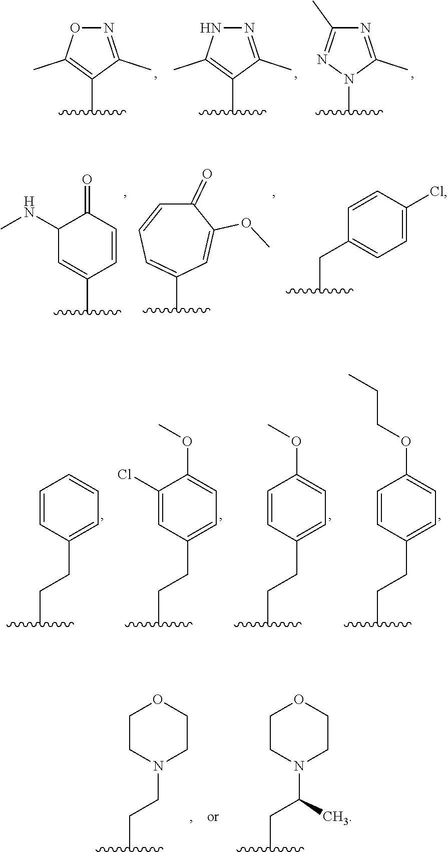

In some embodiments, B-AR is a non-steroidal AR ligand or a steroidal AR ligand.

In some embodiments, B-AR is N-(3-(3-chloro-4-cyanophenoxy)-2,2,4,4-tetramethylcyclobutyl)-aryl-carboxamide or N-(3-(3-trifluroromethyl-4-cyanophenoxy)-2,2,4,4-tetramethylcyclobutyl)-aryl-carboxamide.

In some embodiments, B-AR is flutamide, hydroxylflutamide, nilutamide, bicalutamide, enzalutamide, apalutamide, ODM201, AZD3514, BMS641988, or N-[trans-3-(3-Chloro-4-cyanophenoxy)-2,2,4,4-tetramethylcyclobutyl]-1-(2-hydroxyethyl)-1H-pyrazole-4-carboxamide.

In some embodiments, B-AR comprises ahead group and an optional core, wherein the head group is selected from:

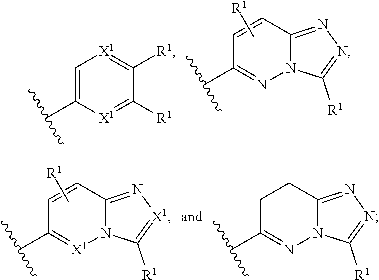

each X¹ is independently —CR¹— or —N—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂.
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

In some embodiments, the head group is selected from:

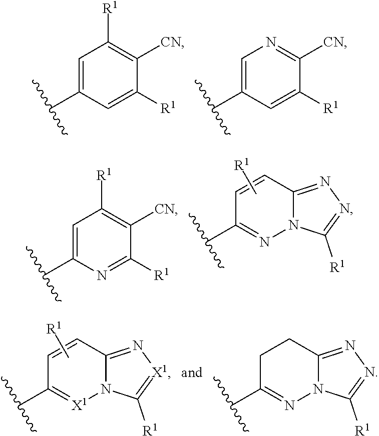

In some embodiments, the head group is selected from:

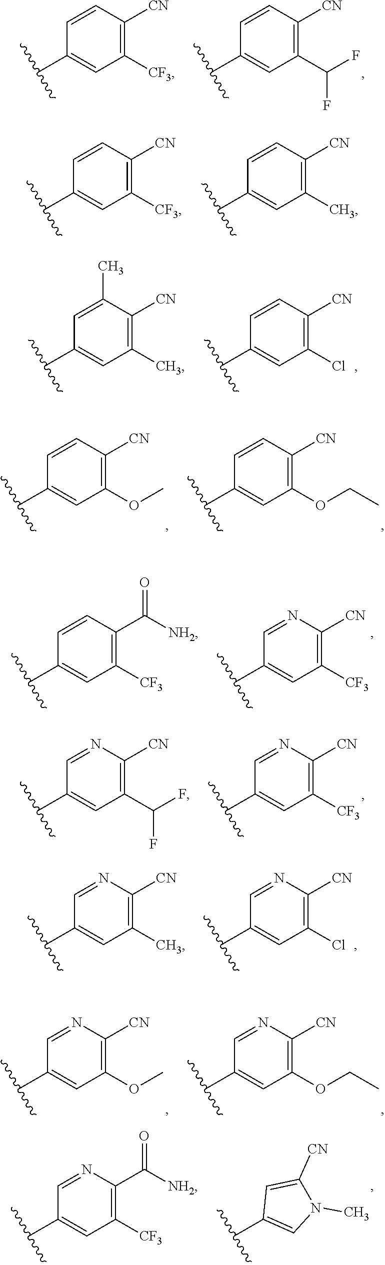

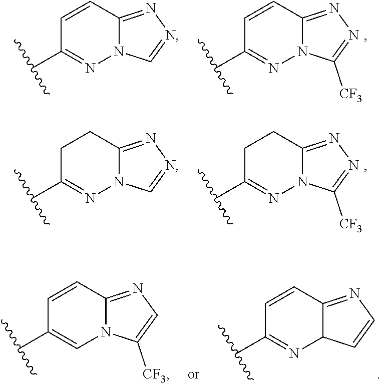

In some embodiments, the optional core comprises a group selected from:

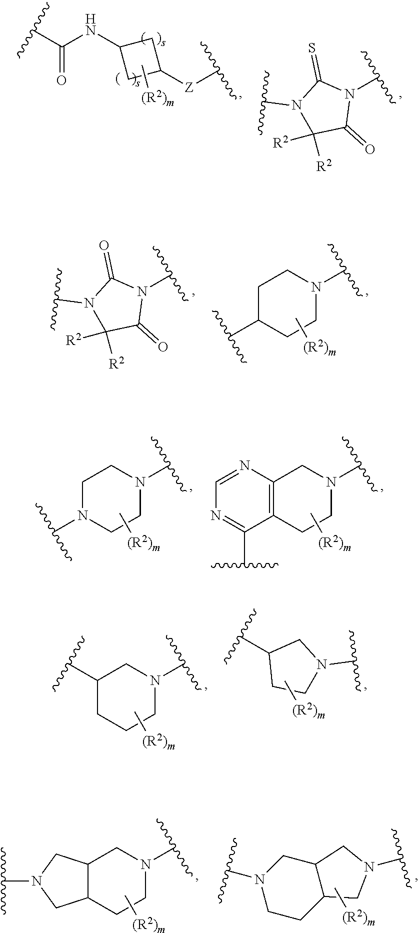

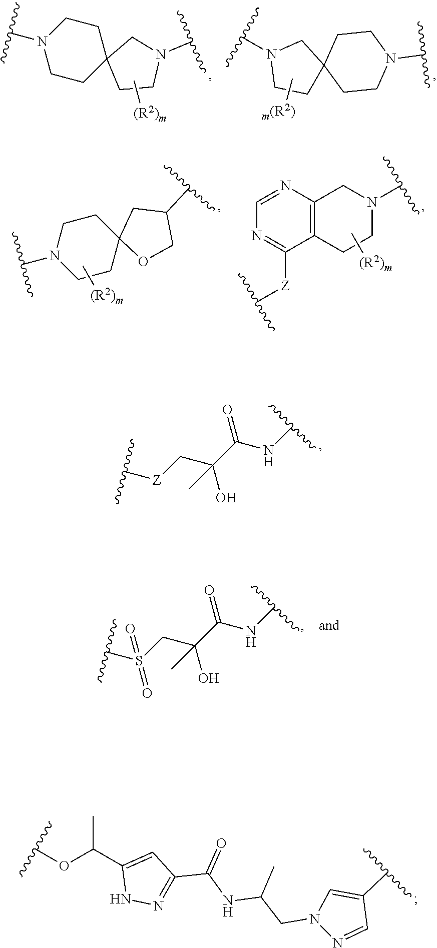

Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈ cycloalkyl or a substituted or unsubstituted monocyclic 3- to 8-membered heterocycloalkyl;
m is 0, 1, 2, 3, or 4;
each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

In some embodiments, the optional core further comprises a ring D that is a 5-, 6-, 8-, 9- or 10-membered aryl or a 5-, 6-, 8-, 9- or 10-membered heteroaryl that is optionally substituted with s R³; each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.

In some embodiments, the optional core comprises a group selected from:

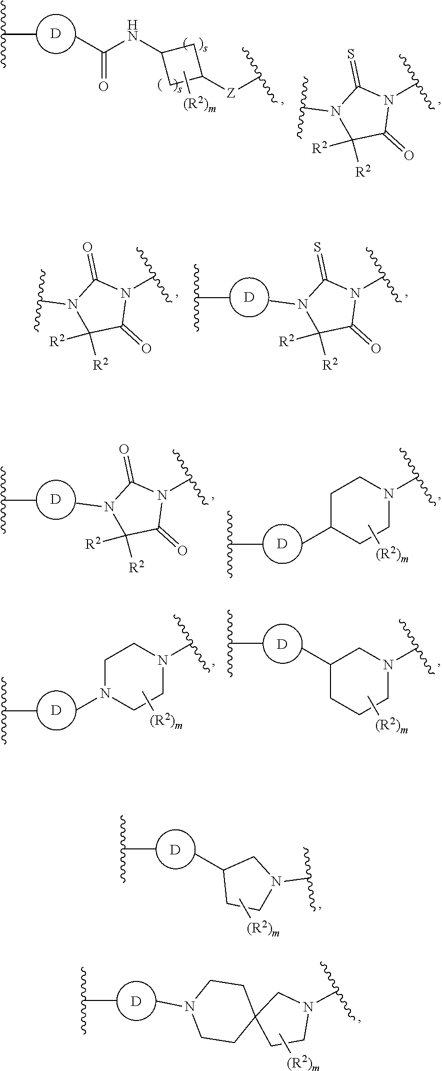

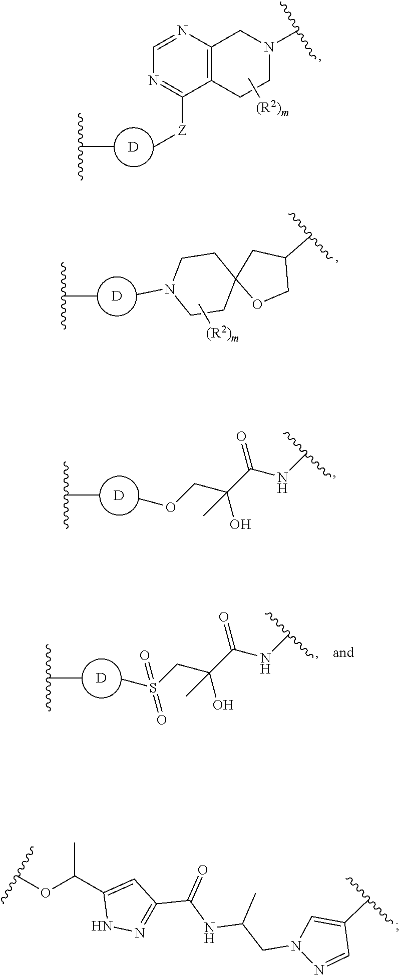

In some embodiments, B-AR has the structure of Formula (IIIa), or a pharmaceutically acceptable salt or solvate thereof:

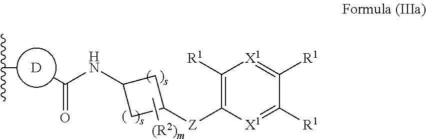

wherein:
each X is independently —CR³— or —N—;
each X¹ is independently —CR¹— or —N—;
Z is —O— or —NR⁵—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂;
ring D that is a 5-, 6-, 8-, 9- or 10-membered aryl or a 5-, 6-, 8-, 9- or 10-membered heteroaryl that is optionally substituted with R³.
R³ is hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂;
n is 0, 1, 2, 3, 4, or 5;
m is 0, 1, 2, 3, or 4;
each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

In some embodiments, ring D is phenyl, naphthyl, pyridinyl, pyrimidinyl, pyridazinyl, pyrazinyl, triazinyl, pyrrolyl, thiazolyl, oxazolyl, isoxazolyl, oxadiazolyl, imidazolyl, pyrazolyl, isothiazolyl, triazolyl, and tetrazolyl, wherein each ring D is optionally substituted substituted with s R³.

In some embodiments, ring D is

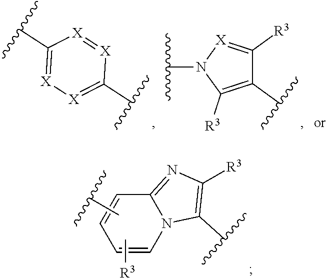

each X is independently —CR³— or —N—.

In some embodiments, ring D is

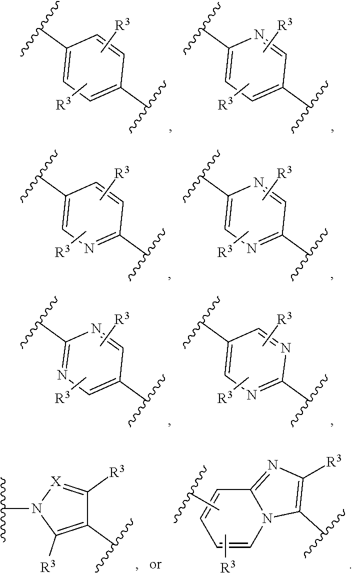

In some embodiments, B-AR has the structure of Formula (IIIa), or a pharmaceutically acceptable salt or solvate thereof:

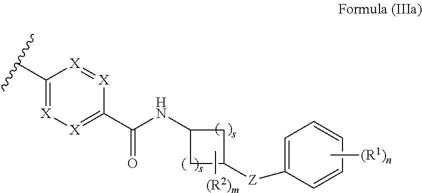

wherein:
each X is independently —CR³— or —N—;
Z is —O— or —NR⁵—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂;
R³ is hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂;
n is 0, 1, 2, 3, 4, or 5;
m is 0, 1, 2, 3, or 4;
each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

In some embodiments, m is 4 and each R² is substituted or unsubstituted C₁-C₆alkyl. In some embodiments, m is 4 and each R² is —CH₃. In some embodiments, two R² on the same carbon atom are taken together with the carbon atom to which they are attached to form a substituted or unsubstituted cyclobutyl, substituted or unsubstituted cyclopentyl, substituted or unsubstituted cyclohexyl, substituted or unsubstituted oxetanyl, substituted or unsubstituted tetrahydrofuranyl, substituted or unsubstituted tetrahydropyranyl, substituted or unsubstituted azetidine, substituted or unsubstituted pyrrolidinyl, or a substituted or unsubstituted piperidinyl. In some embodiments, two R² on the same carbon atom are taken together with the carbon atom to which they are attached to form a substituted or unsubstituted cyclobutyl.

In some embodiments, n is 2 and one R¹ is halogen and the other R¹ is —CN. In some embodiments, n is 2 and one R¹ is —Cl and the other R¹ is —CN.

In some embodiments,

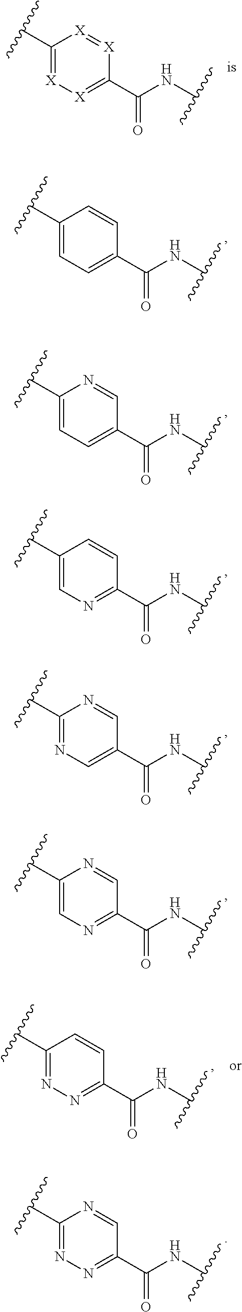

In some embodiments, B-AR has one of the following structures:

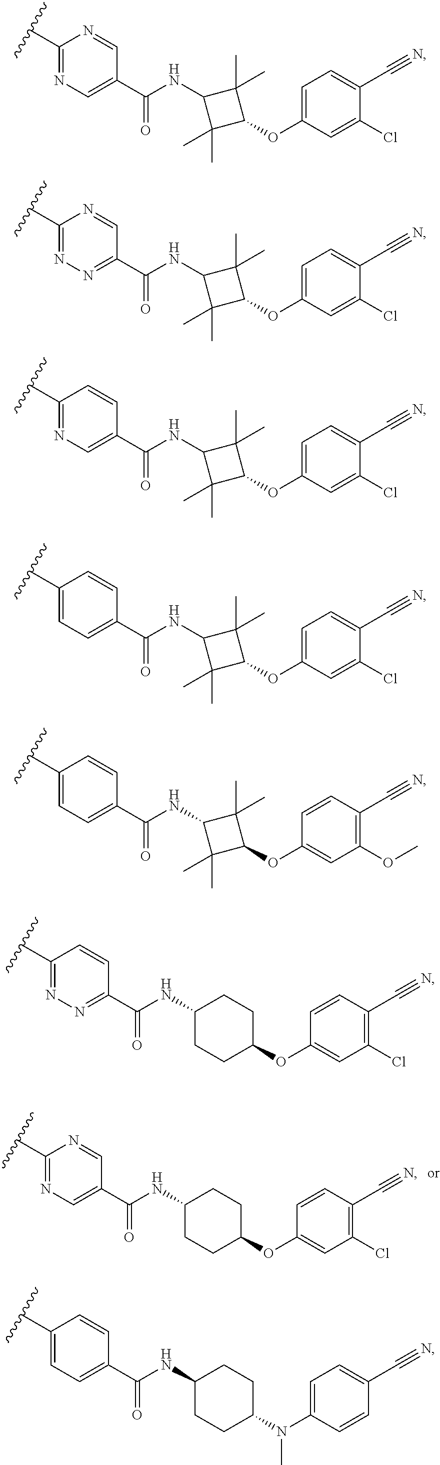

or a pharmaceutically acceptable salt or solvate thereof.

In some embodiments, B—Ar has one of the following structures:

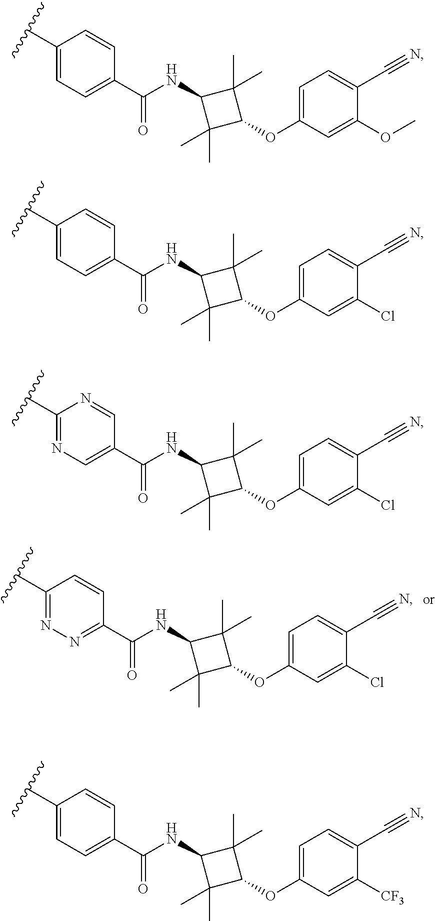 [SMILES: *c1ccc(C(=O)N[C@H]2C(C)(C)[C@H](Oc3ccc(C#N)c(OC)c3)C2(C)C)cc1 ; *c1ccc(C(=O)N[C@H]2C(C)(C)[C@H](Oc3ccc(C#N)c(Cl)c3)C2(C)C)cc1 ; *c1ncc(C(=O)N[C@H]2C(C)(C)[C@H](Oc3ccc(C#N)c(Cl)c3)C2(C)C)cn1 ; *c1ccc(C(=O)N[C@H]2C(C)(C)[C@H](Oc3ccc(C#N)c(Cl)c3)C2(C)C)nn1 ; *c1ccc(C(=O)N[C@H]2C(C)(C)[C@H](Oc3ccc(C#N)c(C(F)(F)F)c3)C2(C)C)cc1]

or a pharmaceutically acceptable salt or solvate thereof.

In some embodiments, the BDP is an AR antagonist. In some embodiments, the BDP is an AR antagonist comprising a N-(3-(3-chloro-4-cyanophenoxy)-2,2,4,4-tetramethylcyclobutyl)-aryl-carboxamide or a N-(3-(3-trifluroromethyl-4-cyanophenoxy)-2,2,4,4-tetramethylcyclobutyl)-aryl-carboxamide. In some embodiments, the BDP is an AR antagonist that is flutamide, hydroxylflutamide, nilutamide, bicalutamide, enzalutamide, apalutamide, ODM201, AZD3514, BMS641988, or N-[trans-3-(3-Chloro-4-cyanophenoxy)-2,2,4,4-tetramethylcyclobutyl]-1-(2-hydroxyethyl)-1H-pyrazole-4-carboxamide.

In some embodiments, the BDP has one of the following structures:

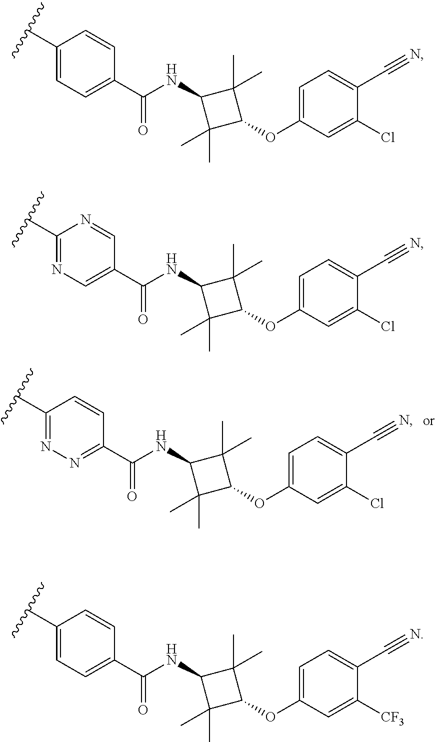 [SMILES: *c1ccc(C(=O)N[C@H]2C(C)(C)[C@H](Oc3ccc(C#N)c(Cl)c3)C2(C)C)cc1 ; *c1ncc(C(=O)N[C@H]2C(C)(C)[C@H](Oc3ccc(C#N)c(Cl)c3)C2(C)C)cn1 ; *c1ccc(C(=O)N[C@H]2C(C)(C)[C@H](Oc3ccc(C#N)c(Cl)c3)C2(C)C)nn1 ; *c1ccc(C(=O)N[C@H]2C(C)(C)[C@H](Oc3ccc(C#N)c(C(F)(F)F)c3)C2(C)C)cc1]

In some embodiments, the BDP has one of the following structures:

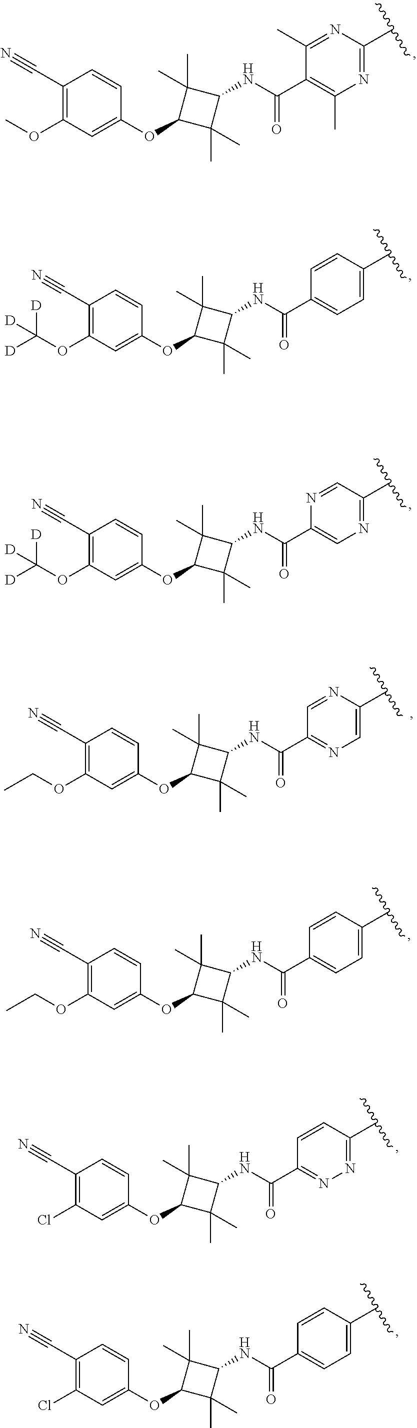

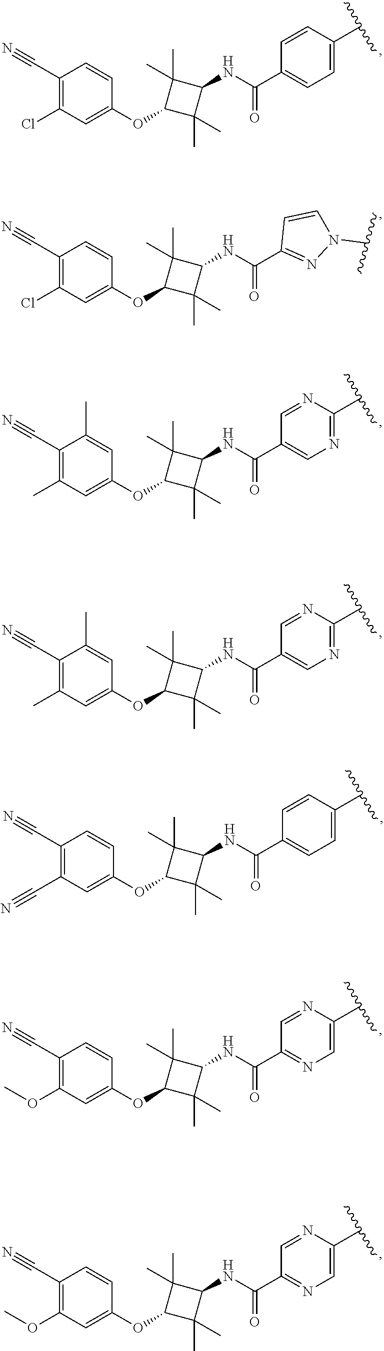

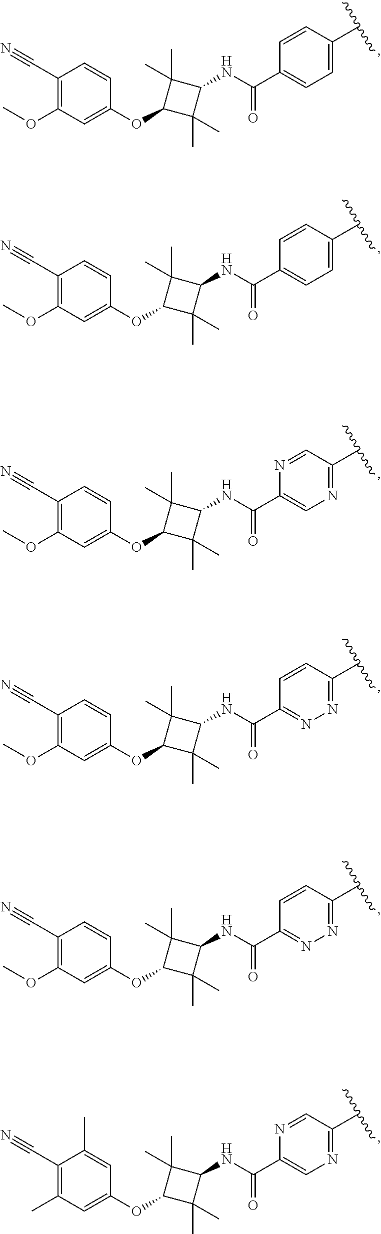 [SMILES: *c1ccc(C(=O)N[C@H]2C(C)(C)[C@H](Oc3ccc(C#N)c(OC)c3)C2(C)C)cc1 ; *c1ccc(C(=O)N[C@H]2C(C)(C)[C@H](Oc3ccc(C#N)c(OC)c3)C2(C)C)cc1 ; *c1cnc(C(=O)N[C@H]2C(C)(C)[C@H](Oc3ccc(C#N)c(OC)c3)C2(C)C)cn1 ; *c1ccc(C(=O)N[C@H]2C(C)(C)[C@H](Oc3ccc(C#N)c(OC)c3)C2(C)C)nn1 ; *c1ccc(C(=O)N[C@H]2C(C)(C)[C@H](Oc3ccc(C#N)c(OC)c3)C2(C)C)nn1 ; *c1cnc(C(=O)N[C@H]2C(C)(C)[C@H](Oc3cc(C)c(C#N)c(C)c3)C2(C)C)cn1]

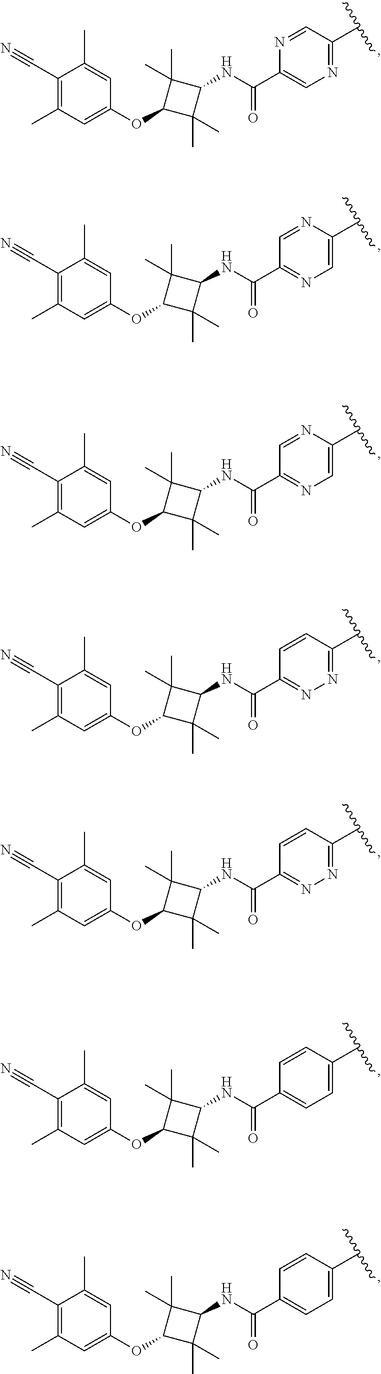

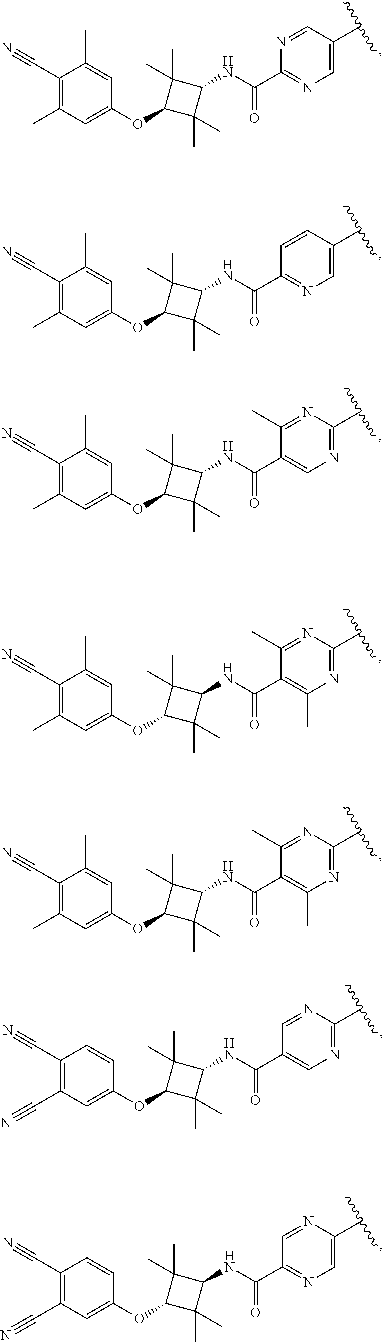

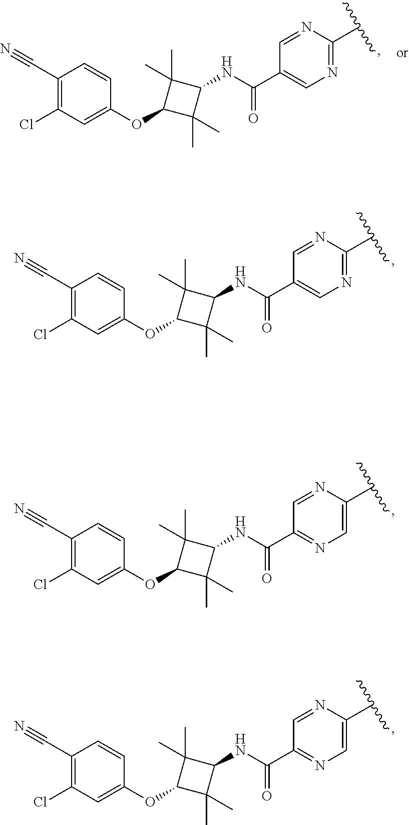 [SMILES: *c1ncc(C(=O)N[C@H]2C(C)(C)[C@H](Oc3ccc(C#N)c(Cl)c3)C2(C)C)cn1 ; *c1ncc(C(=O)N[C@H]2C(C)(C)[C@H](Oc3ccc(C#N)c(Cl)c3)C2(C)C)cn1 ; *c1cnc(C(=O)N[C@H]2C(C)(C)[C@H](Oc3ccc(C#N)c(Cl)c3)C2(C)C)cn1 ; *c1cnc(C(=O)N[C@H]2C(C)(C)[C@H](Oc3ccc(C#N)c(Cl)c3)C2(C)C)cn1]

or a pharmaceutically acceptable salt or solvate thereof.

In some embodiments, the linker is absent.

In some embodiments, the linker has a prescribed length thereby linking the SBDDP and the BDP while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the use of trivalent linkers allow for the preparation of heterotrifunctional compounds comprising one silent binder to a DDP and two binders to a disease protein, or two silent binders to a DDP and one binder to a disease protein, thereby simultaneously binding a disease protein and a DDP and forming a productive ternary complex. In some embodiments, such heterotrifunctional compounds would result in more sustained and more potent anticancer activity.

In some embodiments, the linker comprises one or more linear structures, one or more non-linear structures, one or more branched structures, one or more cyclic structures, one or more flexible moieties, one or more rigid moieties, or combinations thereof.

In some embodiments, the linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids. In some embodiments, the linker comprises a peptide linkage. The peptide linkage comprises L-amino acids and/or D-amino acids.

In some embodiments, the linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, the linker comprises flexible and/or rigid regions.

In some embodiments, the linker is L, wherein L is absent or,

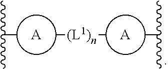

In some embodiments, L is absent. In some embodiments, L is

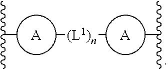

In some embodiments, L comprises substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted 3- to 10-membered heterocycloalkyl, substituted or unsubstituted aryl, substituted or unsubstituted heteroaryl, or combinations thereof.

In some embodiments, L is absent or

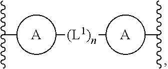

wherein:
each A is independently absent, substituted or unsubstituted monocyclic C₃-C₁₀cycloalkyl, substituted or unsubstituted bridged bicyclic C₃-C₁₀cycloalkyl, substituted or unsubstituted fused bicyclic C₃-C₁₀cycloalkyl, substituted or unsubstituted spiro bicyclic C₃-C₁₀cycloalkyl, substituted or unsubstituted monocyclic 3- to 10-membered heterocycloalkyl, substituted or unsubstituted bridged bicyclic 3- to 10-membered heterocycloalkyl, substituted or unsubstituted fused bicyclic 3- to 10-membered heterocycloalkyl, substituted or unsubstituted spiro bicyclic 3- to 10-membered heterocycloalkyl, substituted or unsubstituted aryl, or substituted or unsubstituted heteroaryl, wherein each A is independently unsubstituted or substituted with x R^{2b};
each L¹ is independently absent,

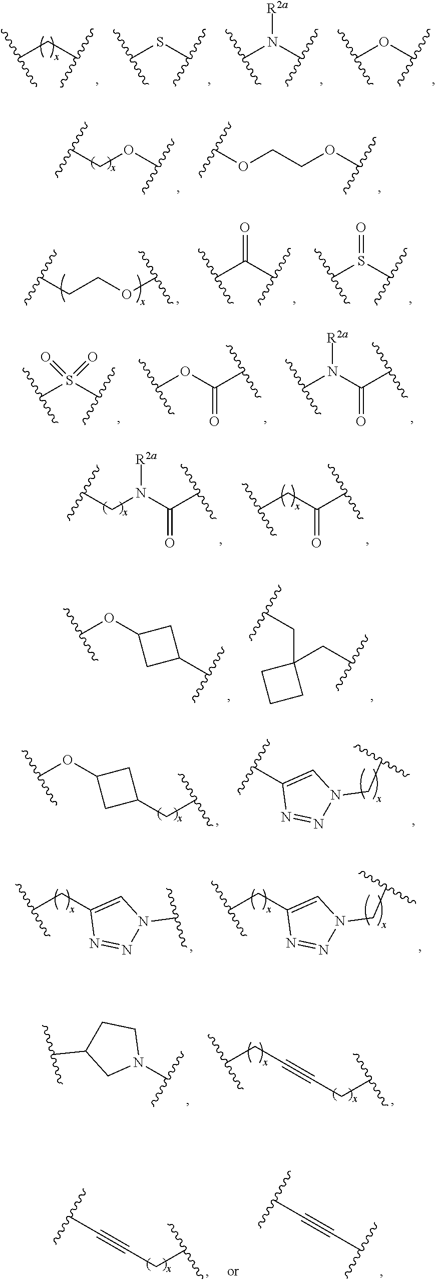

wherein each L¹ is independently unsubstituted or substituted with x R^{2b};
n is 1, 2, 3, 4, 5, or 6;
each x is independently 1, 2, 3, 4, 5, 6, 7, or 8;
each R^{2a} is independently hydrogen or substituted or unsubstituted C₁-C₆alkyl; and
each R^{2b} is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, —OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b});
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, each A is independently absent,

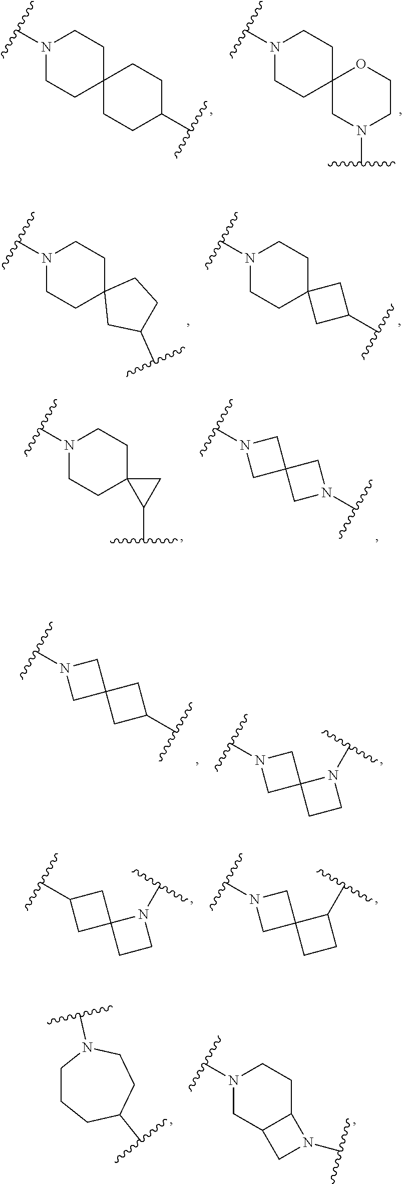

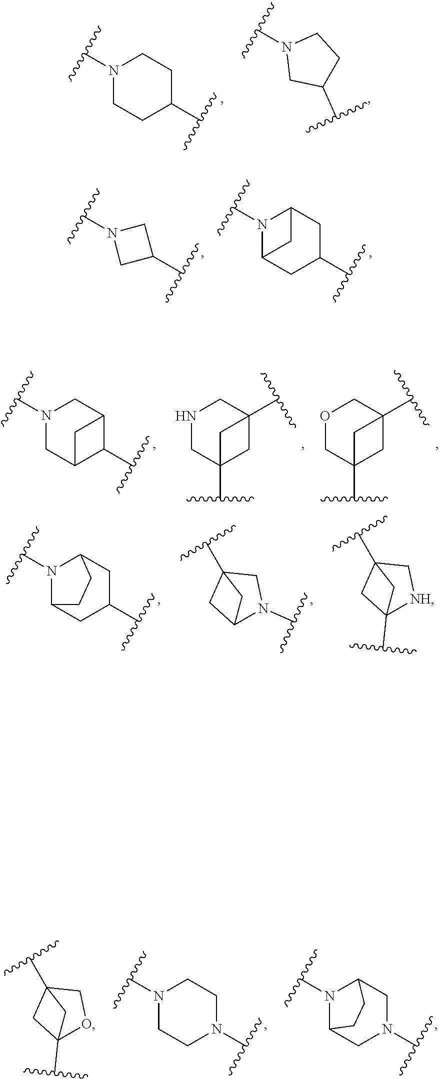

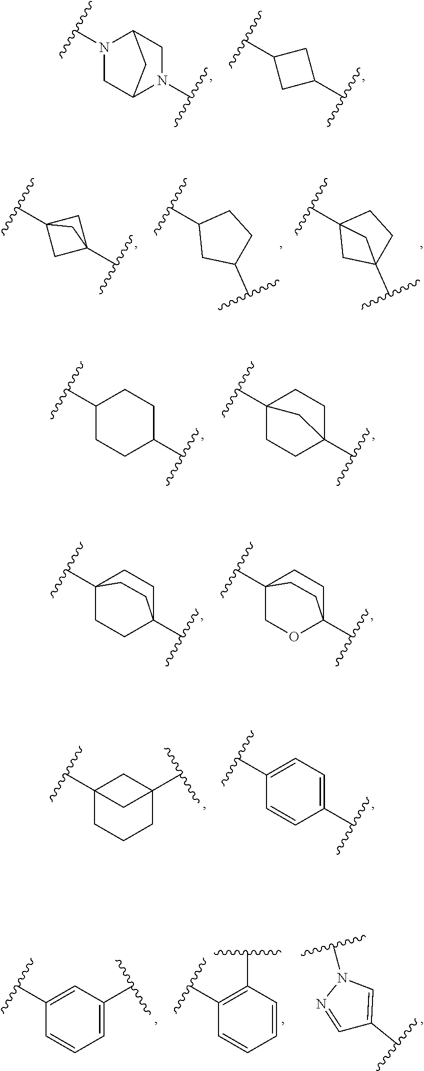

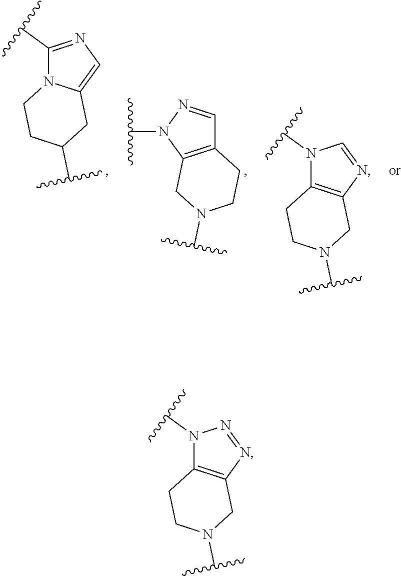

wherein each A is independently unsubstituted or substituted with x R^{2b}. (L¹)ₙ is absent,

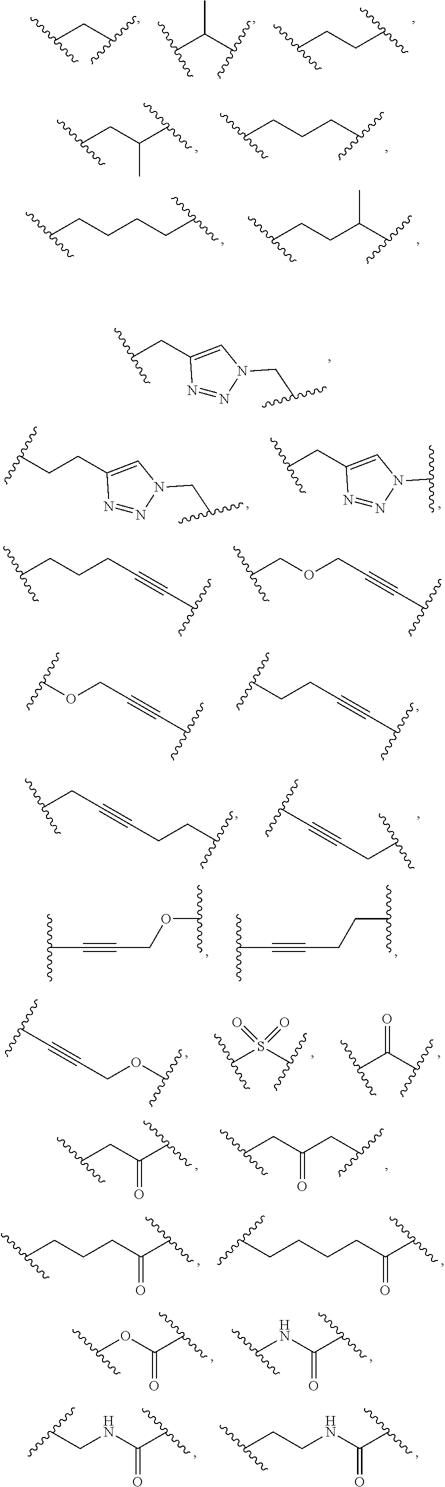

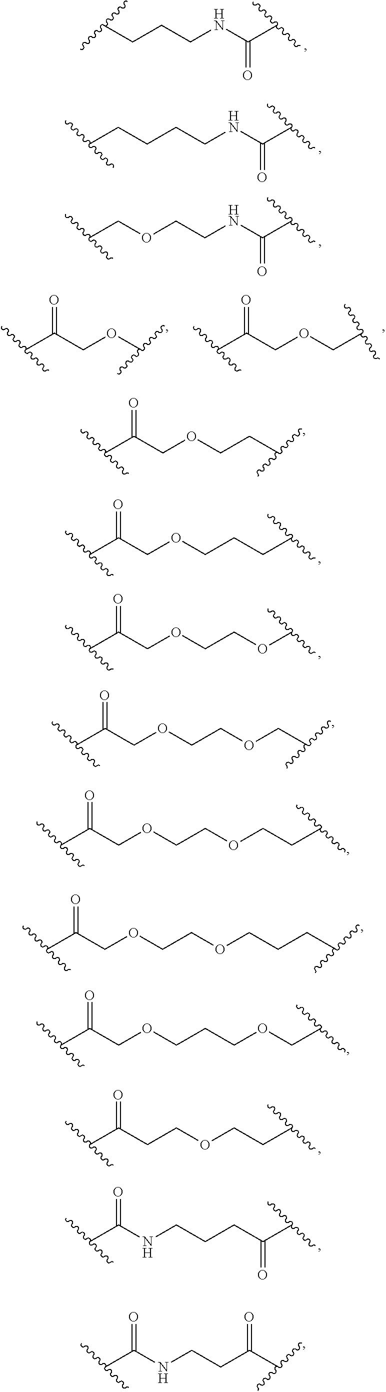

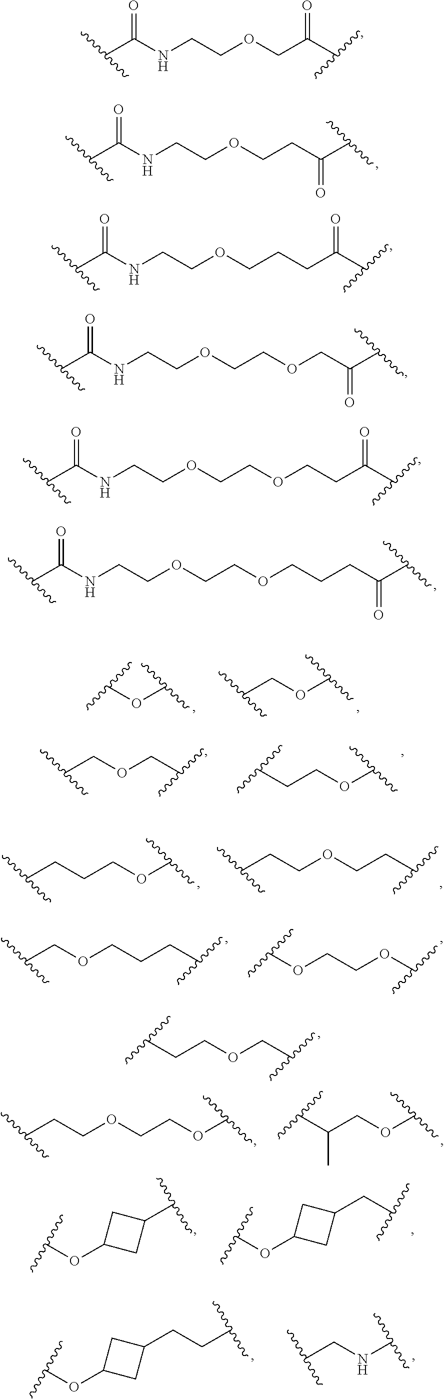

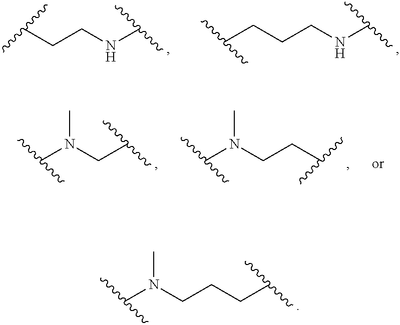

In some embodiments, L is absent or

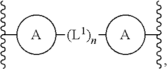

wherein:
each A is independently absent,

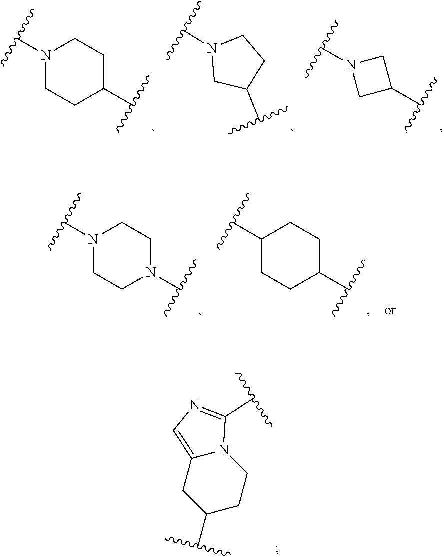 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]

each L¹ is independently absent,

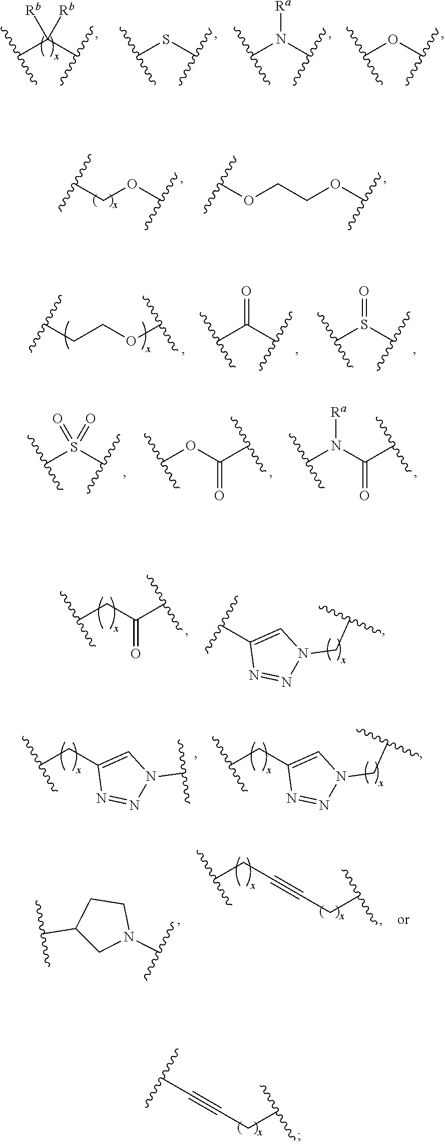

n is 1, 2, or 3;
each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

In some embodiments, each A is independently absent,

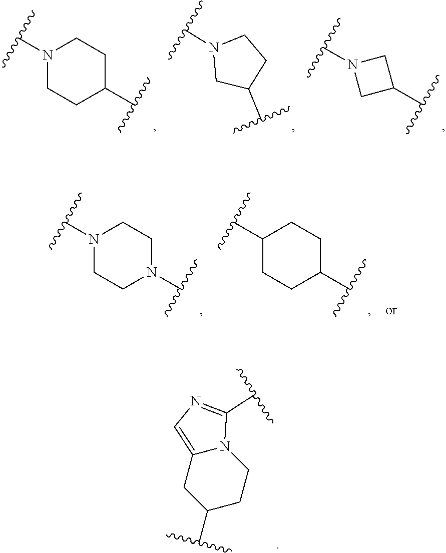 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]

In some embodiments, each A is independently

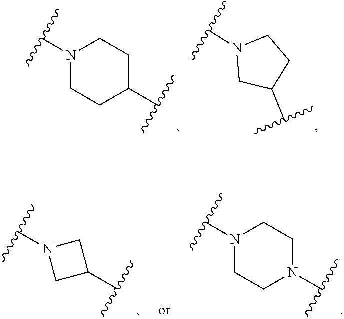 [SMILES: *C1CCN(*)CC1 ; *C1CCN(*)C1 ; *C1CN(*)C1 ; *N1CCN(*)CC1]

In some embodiments, each A is independently absent,

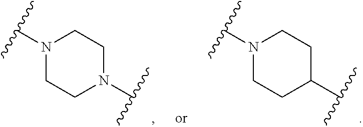 [SMILES: *N1CCN(*)CC1 ; *C1CCN(*)CC1]

In some embodiments, each L¹ is absent,

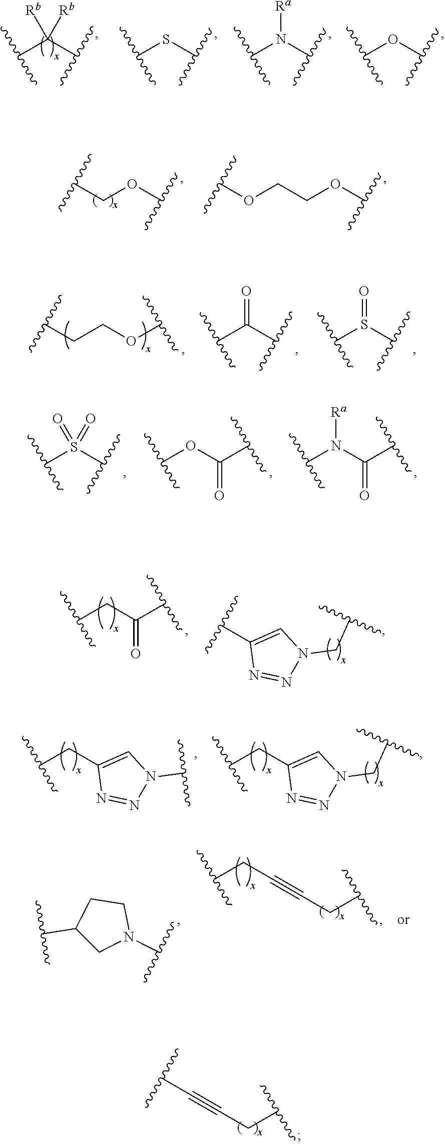

n is 1, 2, or 3;
each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

In some embodiments, n is 1. In some embodiments, n is 2. In some embodiments, n is 3.

In some embodiments, x is 1. In some embodiments, x is 2. In some embodiments, x is 3. In some embodiments, x is 4. In some embodiments, x is 5. In some embodiments, x is 6.

In some embodiments, R^{a} is hydrogen. In some embodiments, each R^{a} is independently substituted or unsubstituted C₁-C₆alkyl. In some embodiments, each R^{a} is independently hydrogen, methyl, ethyl, propyl, iso-propyl, n-butyl, iso-butyl, or tert-butyl. In some embodiments, each R^{a} is independently hydrogen or methyl. In some embodiments, R^{a} is methyl.

In some embodiments, R^{b} is hydrogen. In some embodiments, each R^{b} is independently substituted or unsubstituted C₁-C₆alkyl. In some embodiments, each R^{b} is independently hydrogen, methyl, ethyl, propyl, iso-propyl, n-butyl, iso-butyl, or tert-butyl. In some embodiments, each R^{b} is independently hydrogen or methyl. In some embodiments, R^{b} is methyl.

In some embodiments (L¹)ₙ is absent

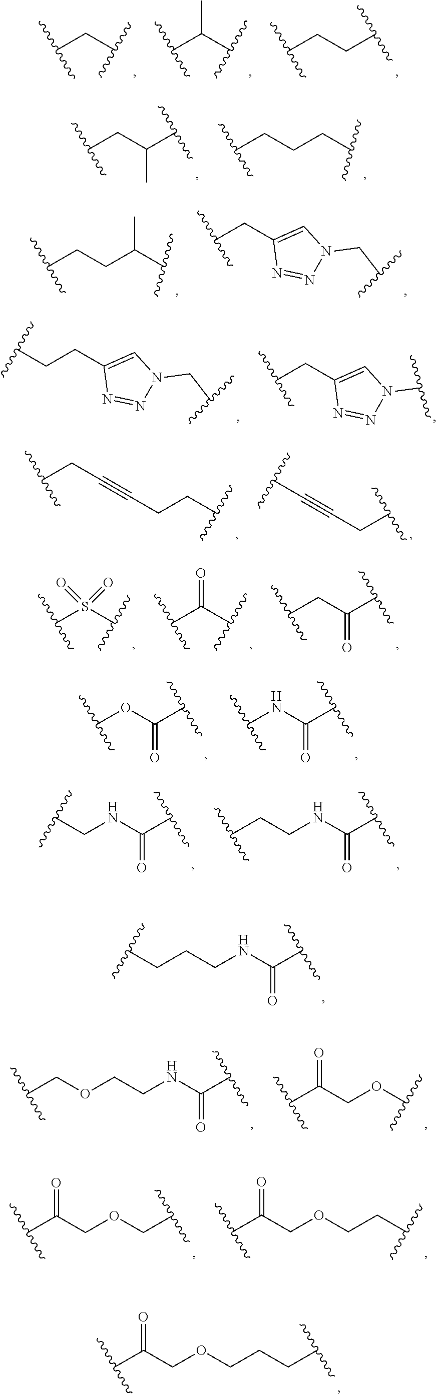

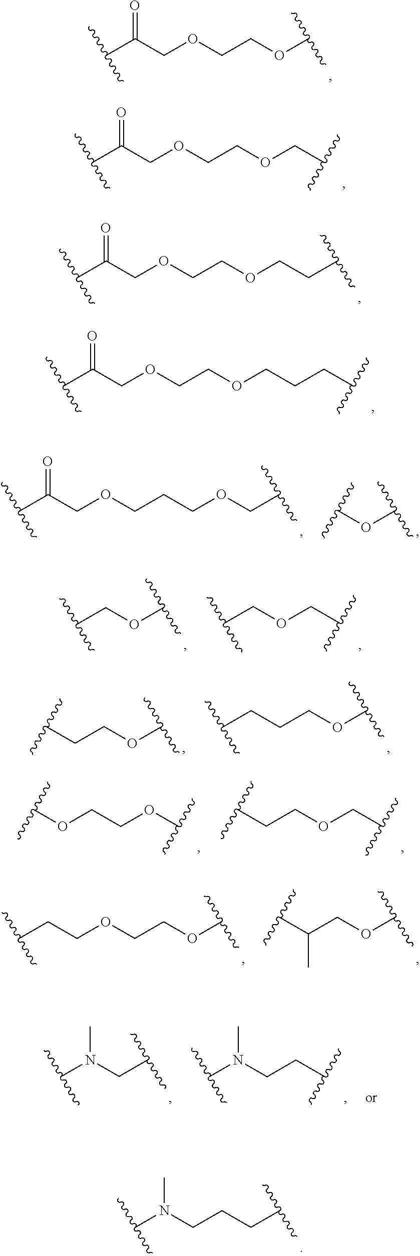

In some embodiments, (L¹)ₙ is absent, or

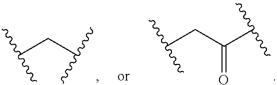

In some embodiments, L is

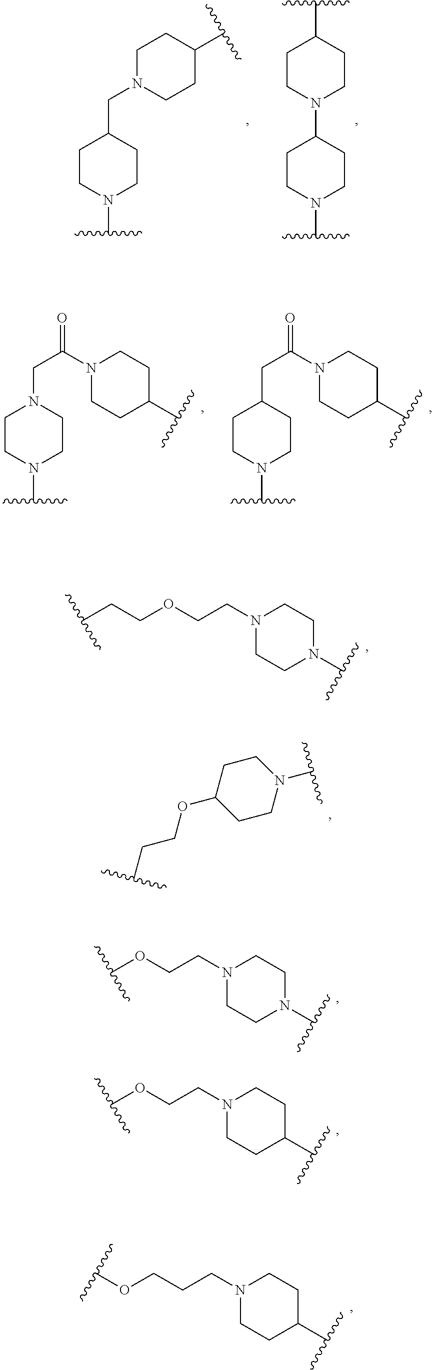

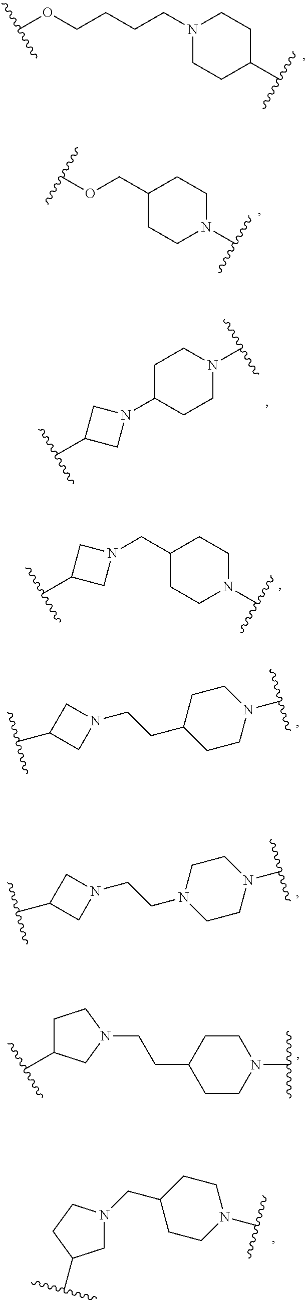

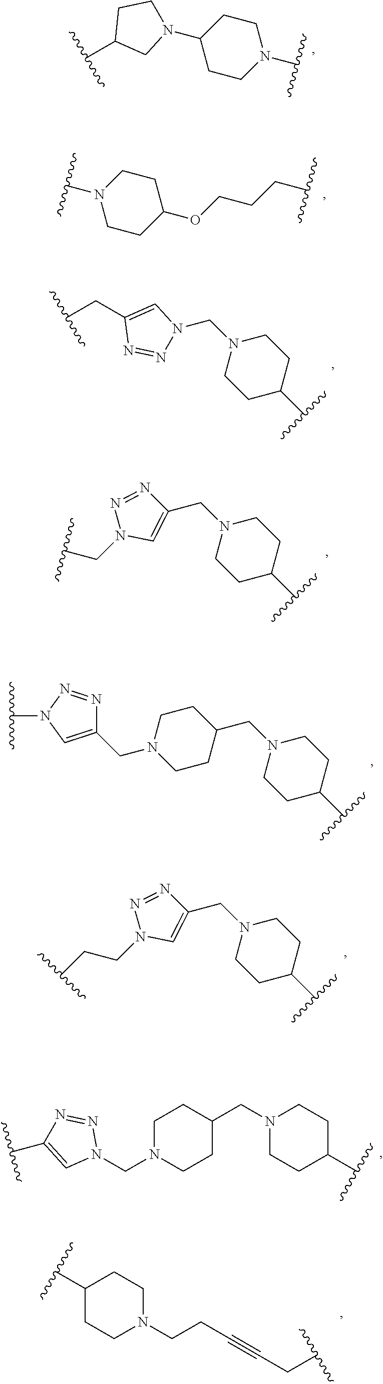

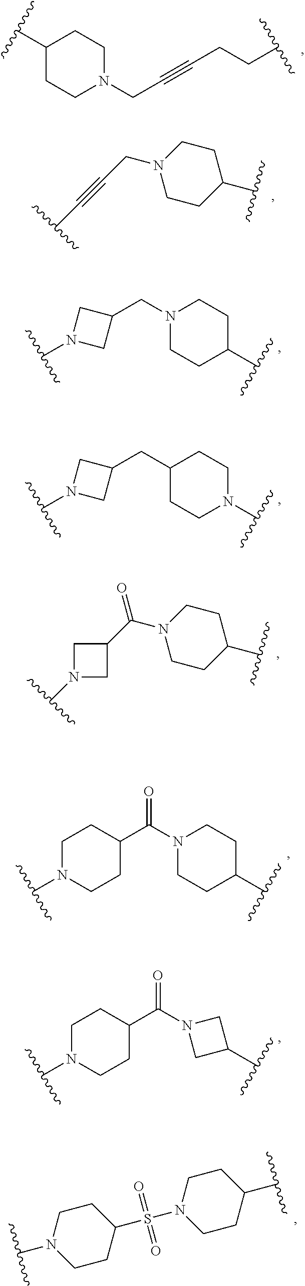

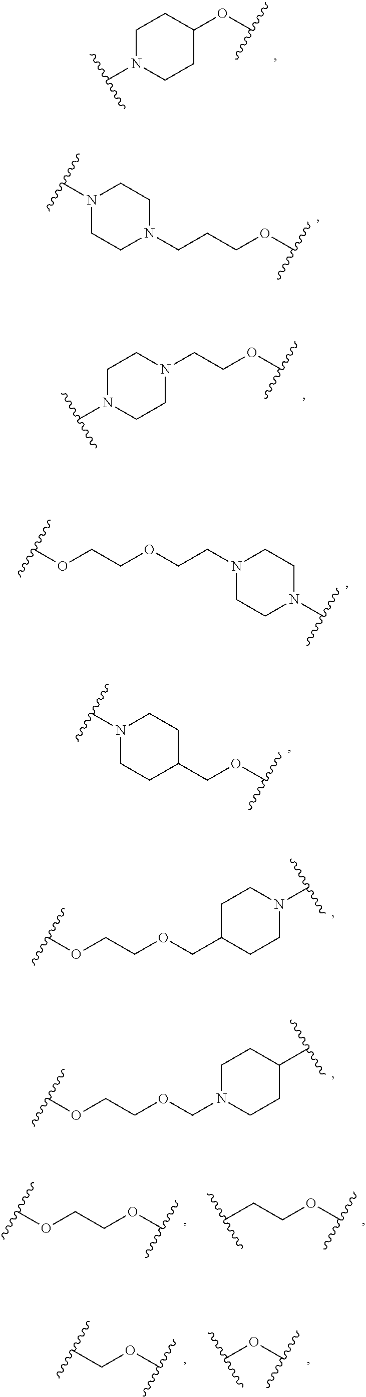

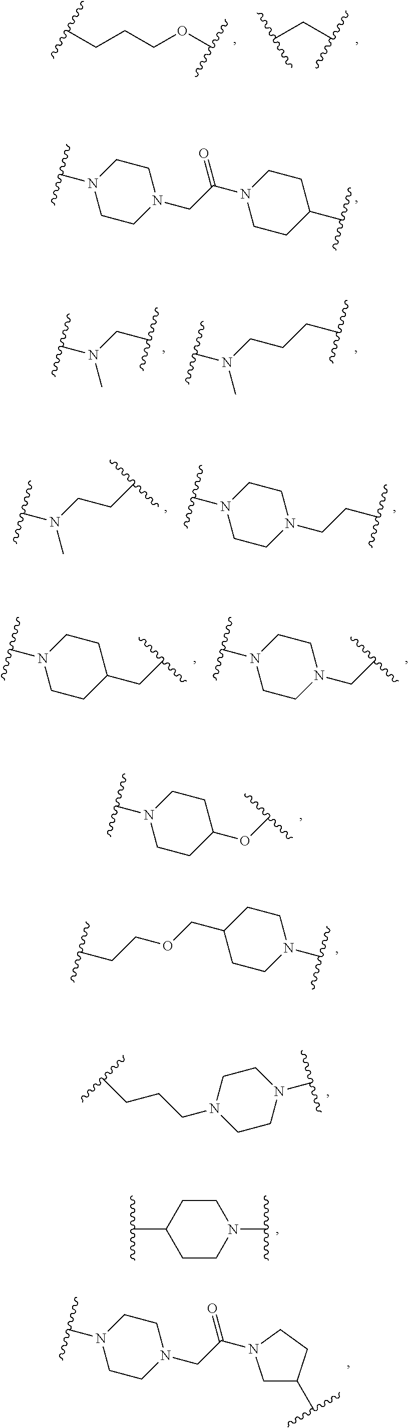

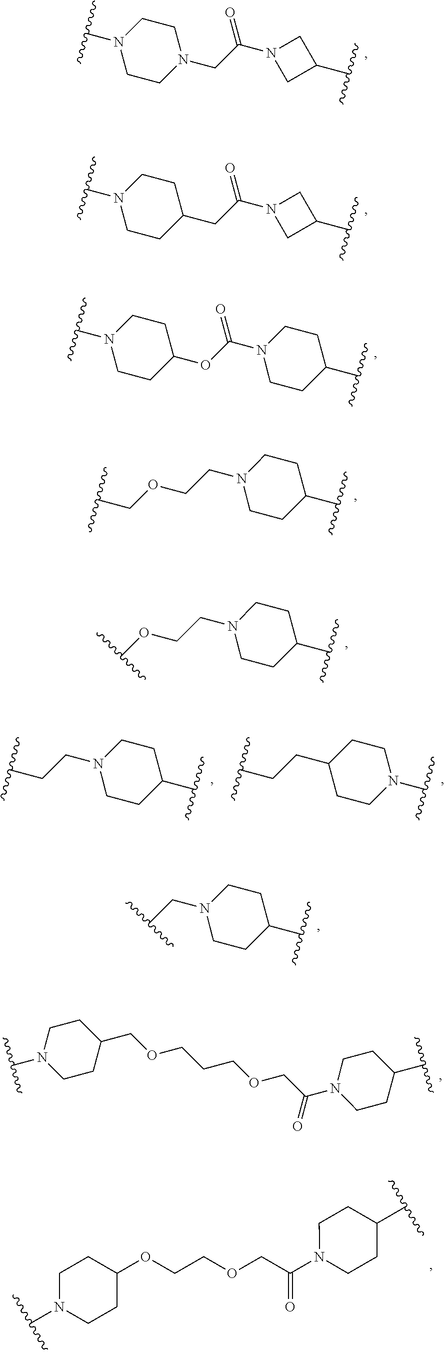

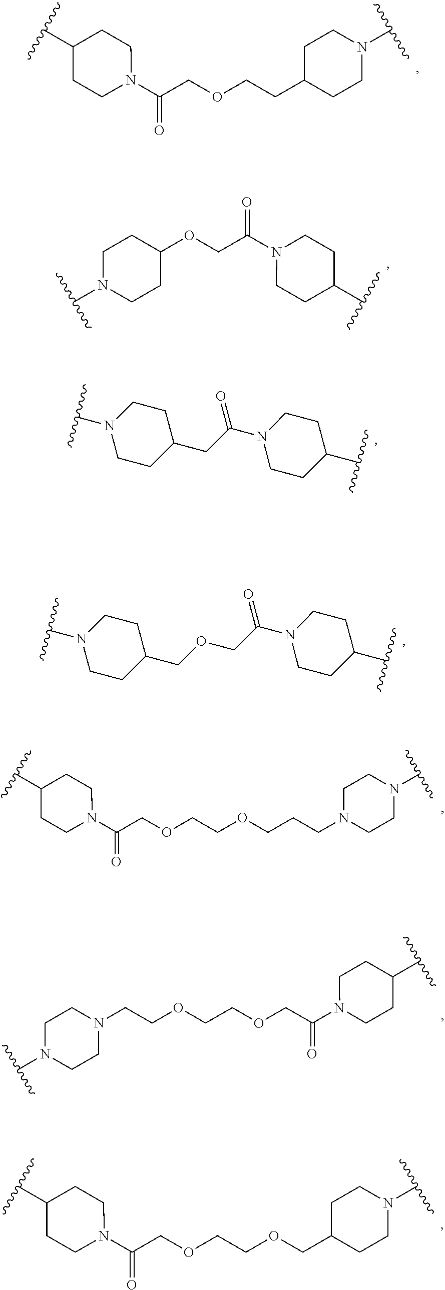

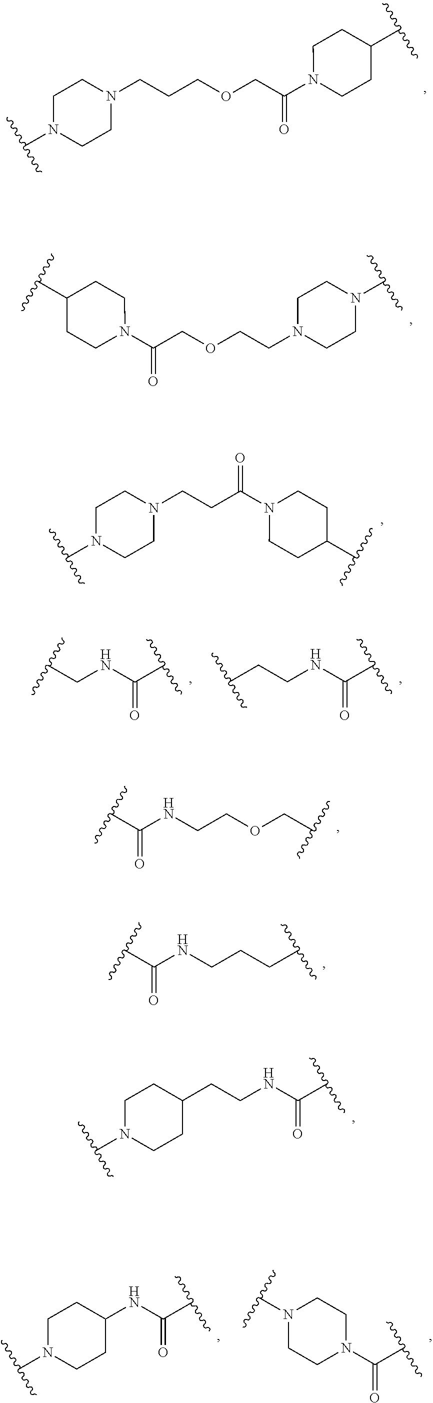

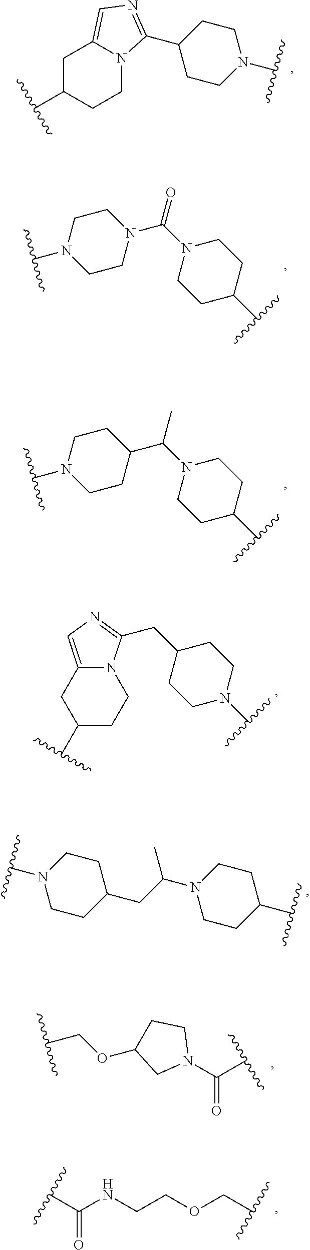

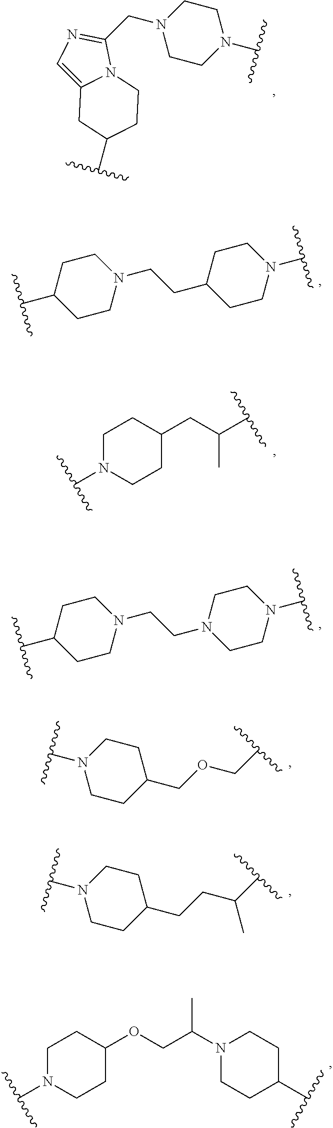

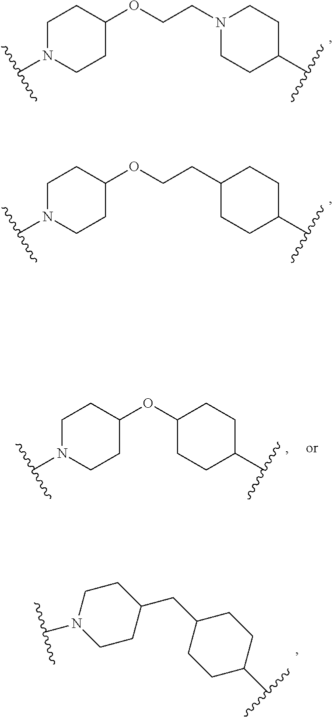 [SMILES: *C1CCN(CCOC2CCN(*)CC2)CC1 ; *C1CCC(CCOC2CCN(*)CC2)CC1 ; *C1CCC(OC2CCN(*)CC2)CC1 ; *C1CCC(CC2CCN(*)CC2)CC1]

or a pharmaceutically acceptable salt or solvate thereof.

In some embodiments, L is

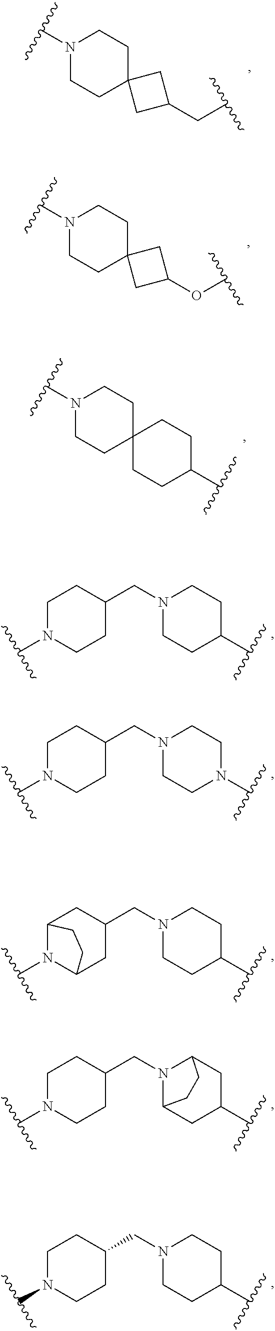

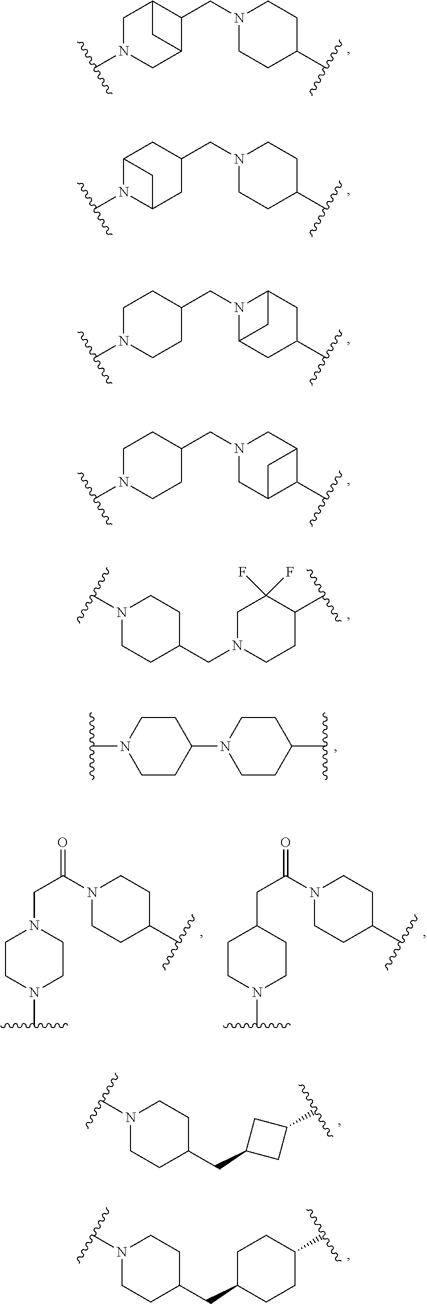

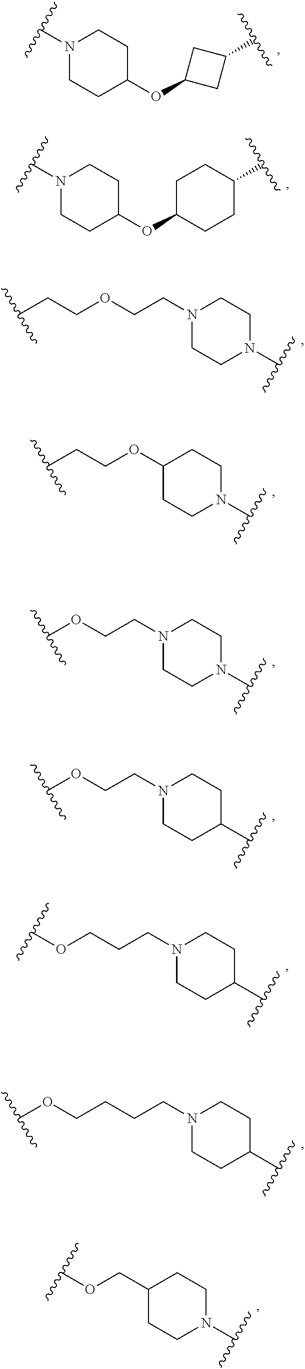

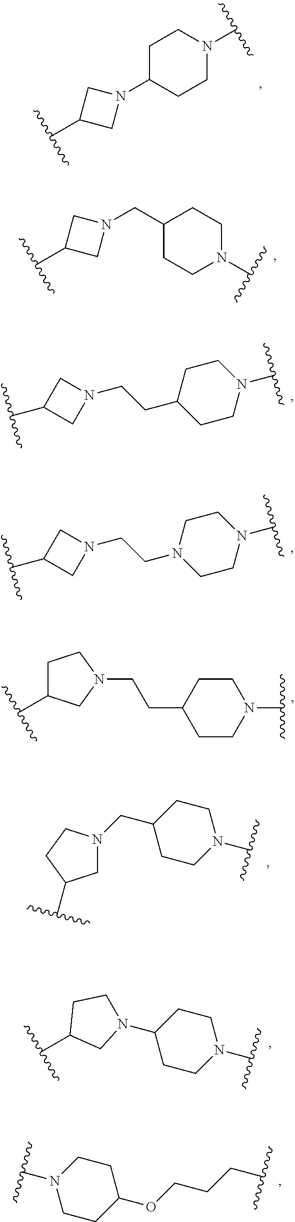

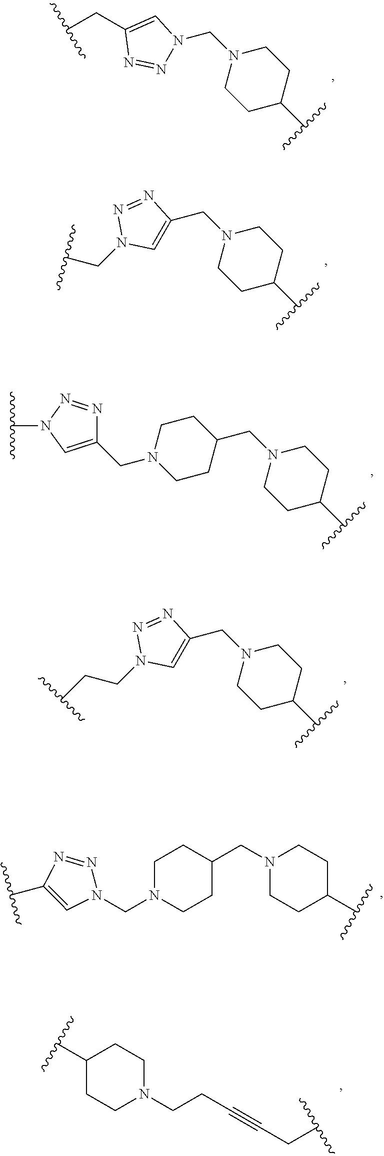 [SMILES: *Cc1cn(CN2CCC(*)CC2)nn1 ; *Cn1cc(CN2CCC(*)CC2)nn1 ; *C1CCN(CC2CCN(Cc3cn(*)nn3)CC2)CC1 ; *CCn1cc(CN2CCC(*)CC2)nn1 ; *c1cn(CN2CCC(CN3CCC(*)CC3)CC2)nn1 ; *CC#CCCN1CCC(*)CC1]

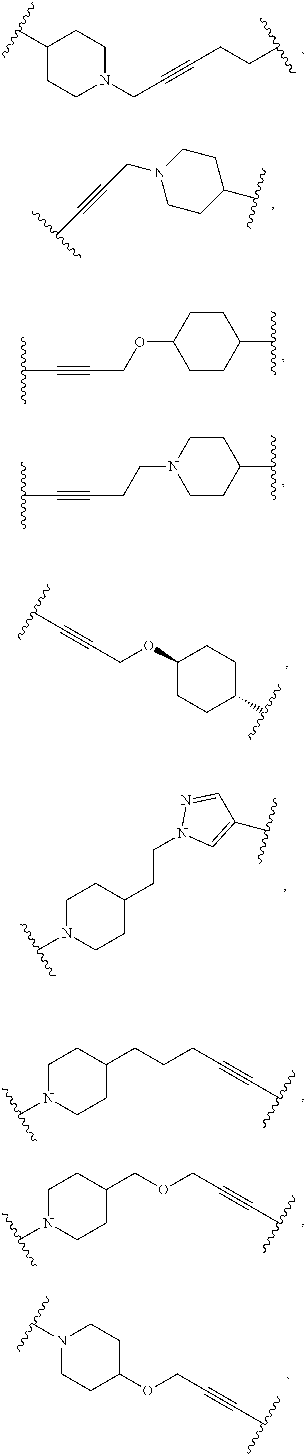

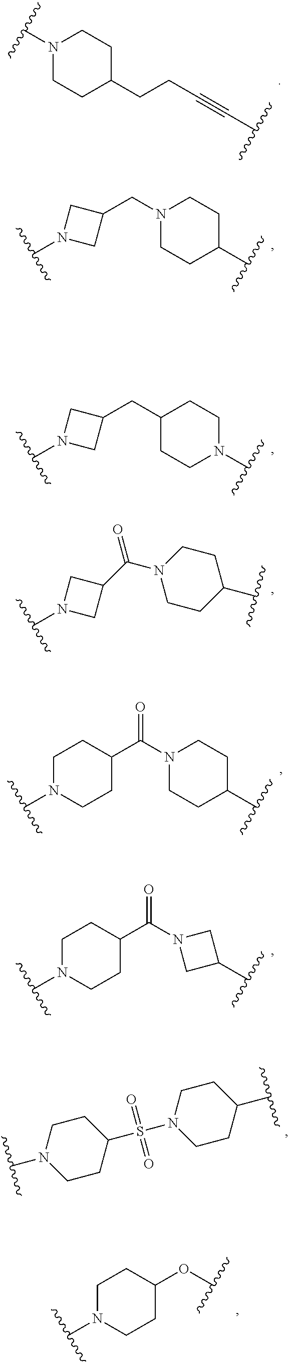

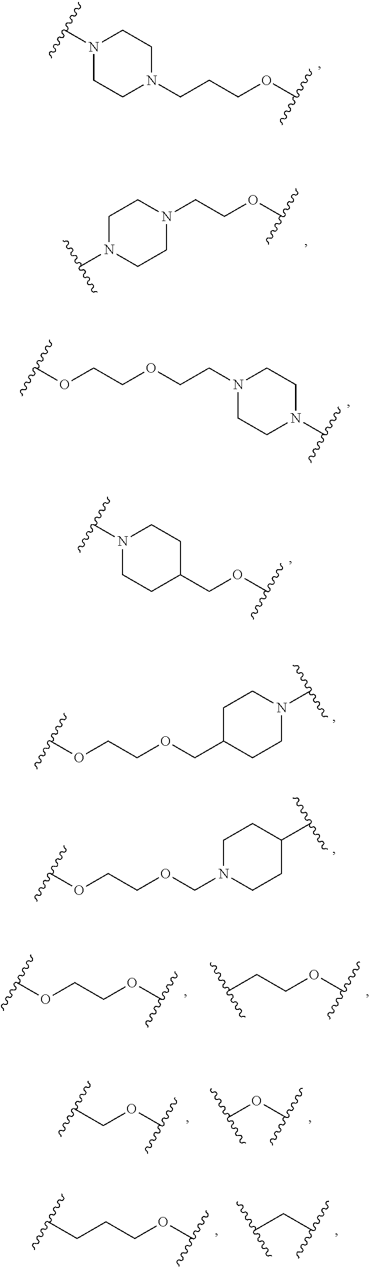

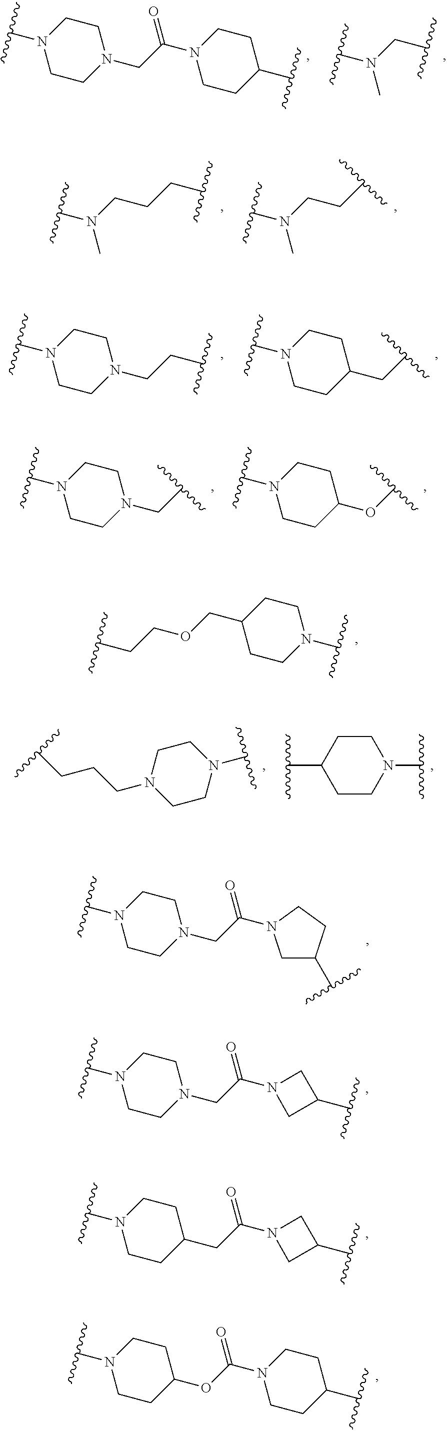

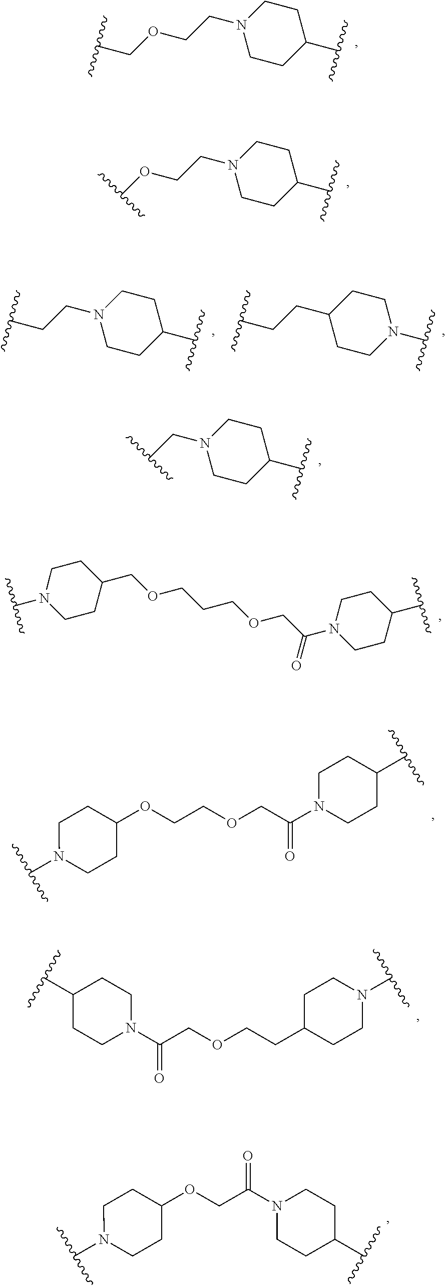

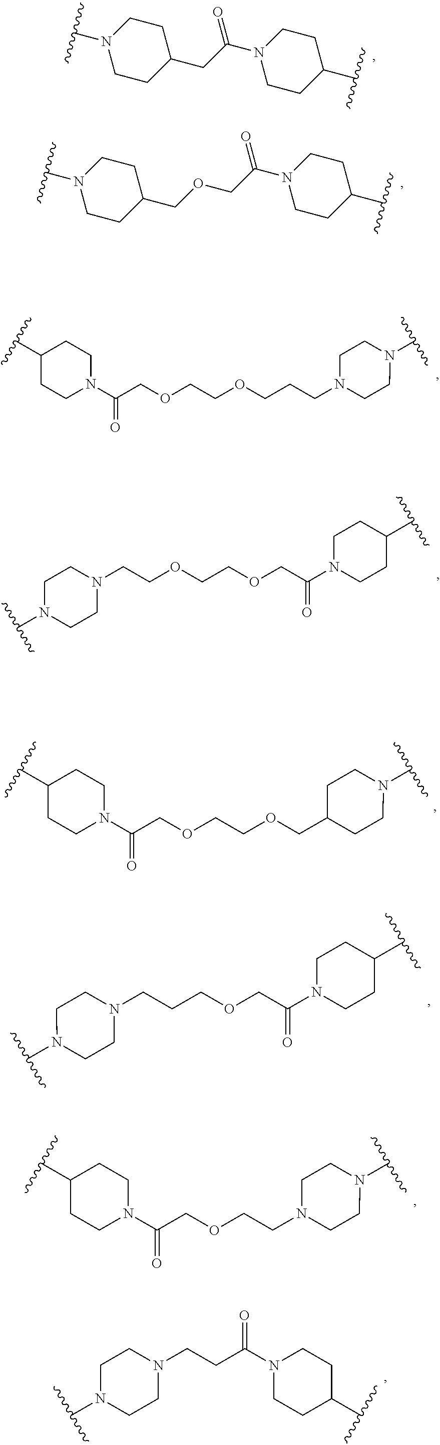

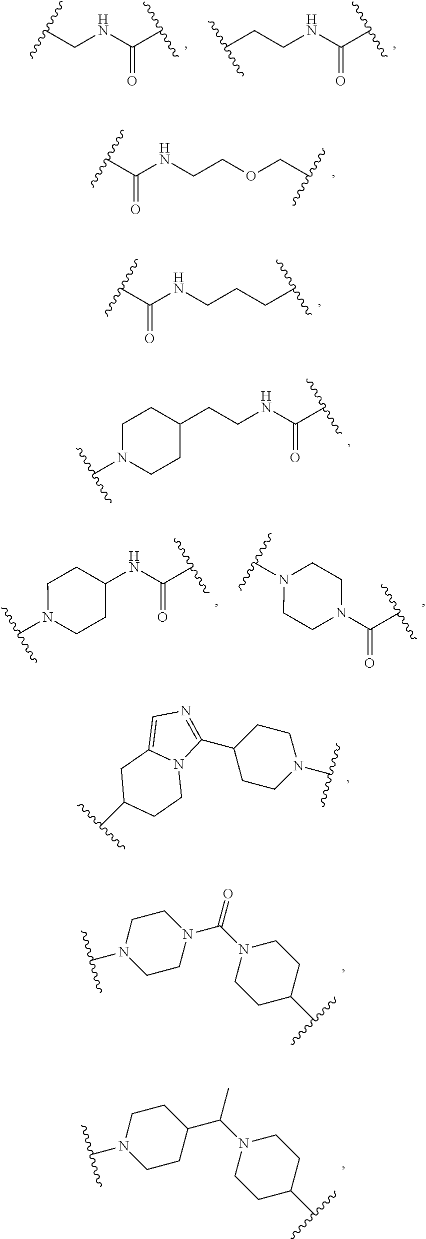

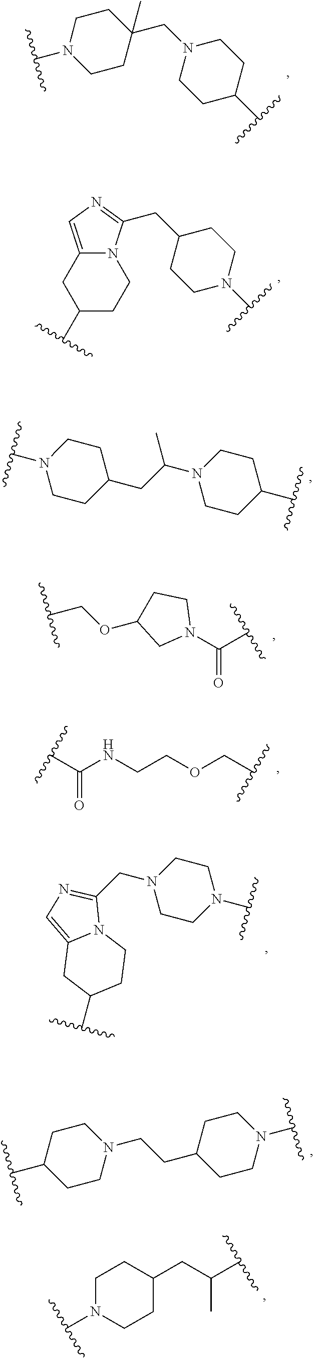

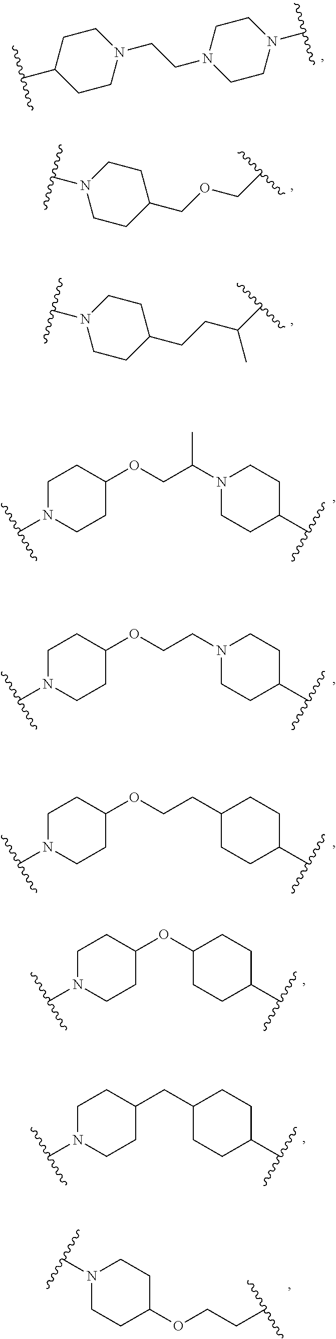

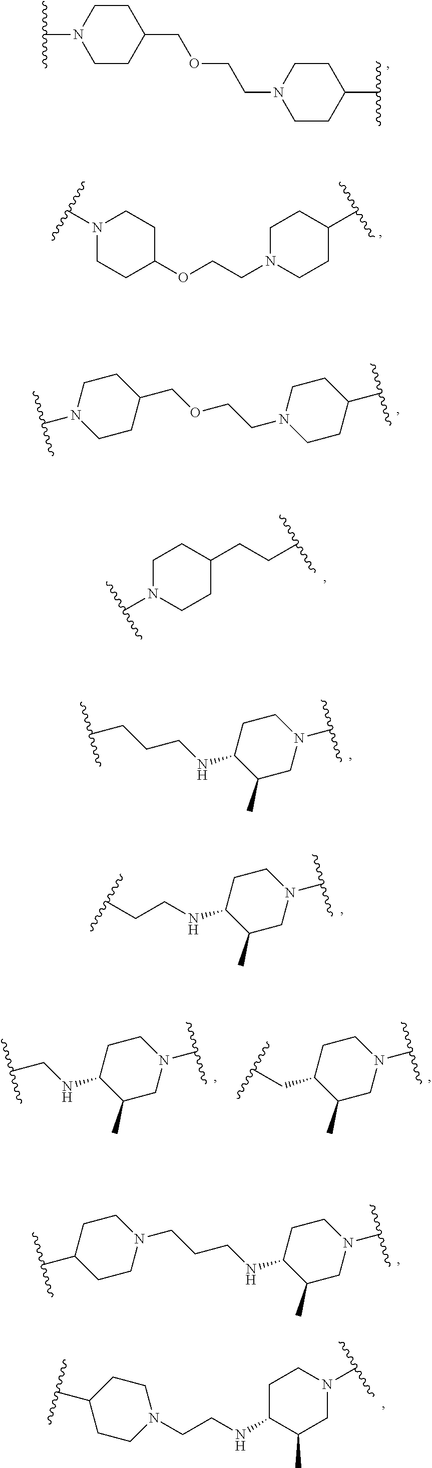

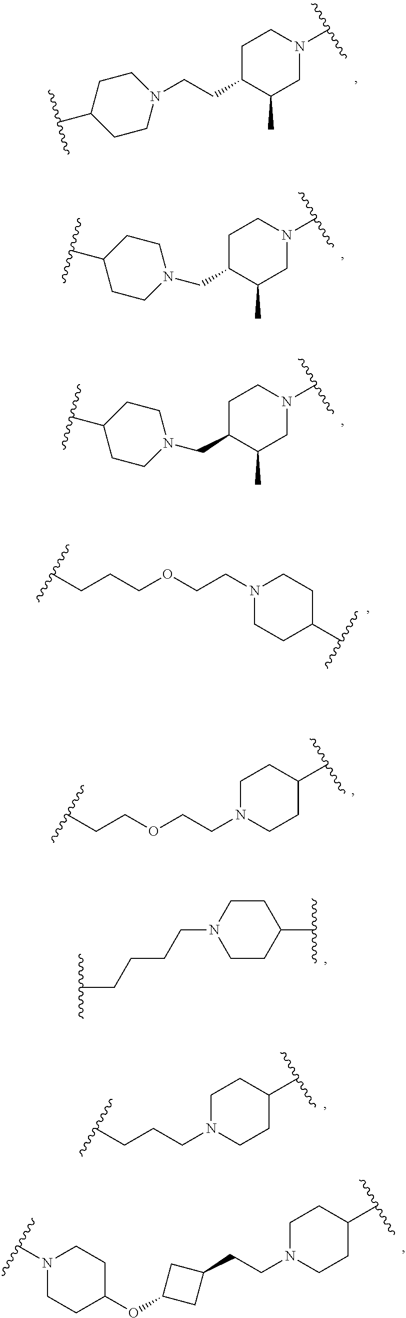

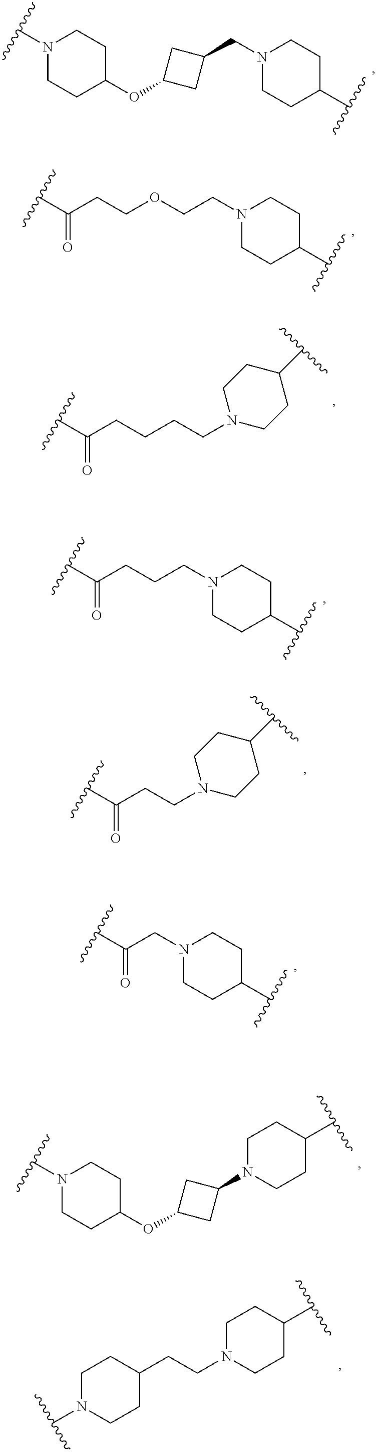

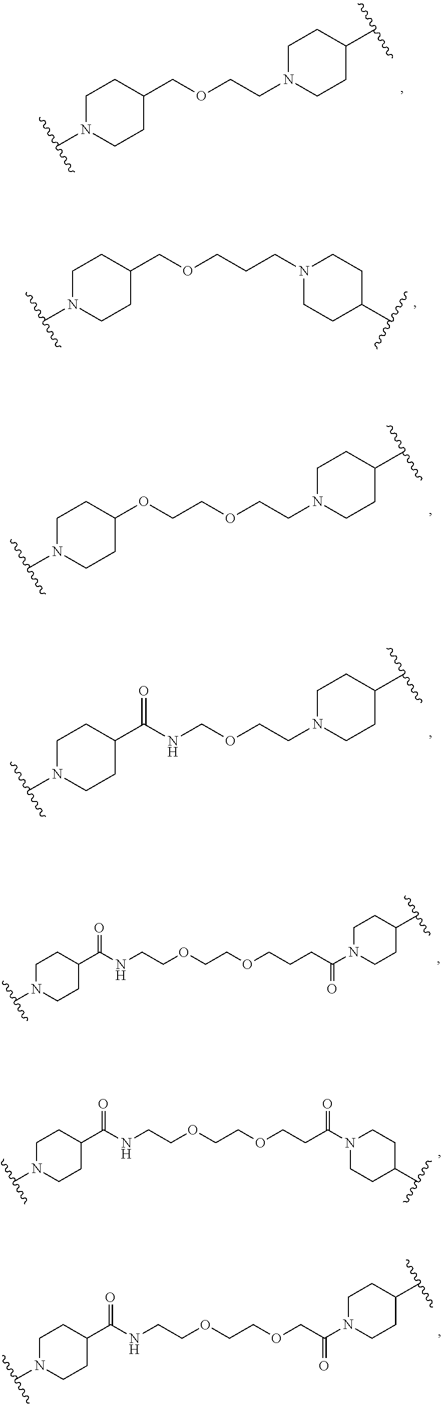

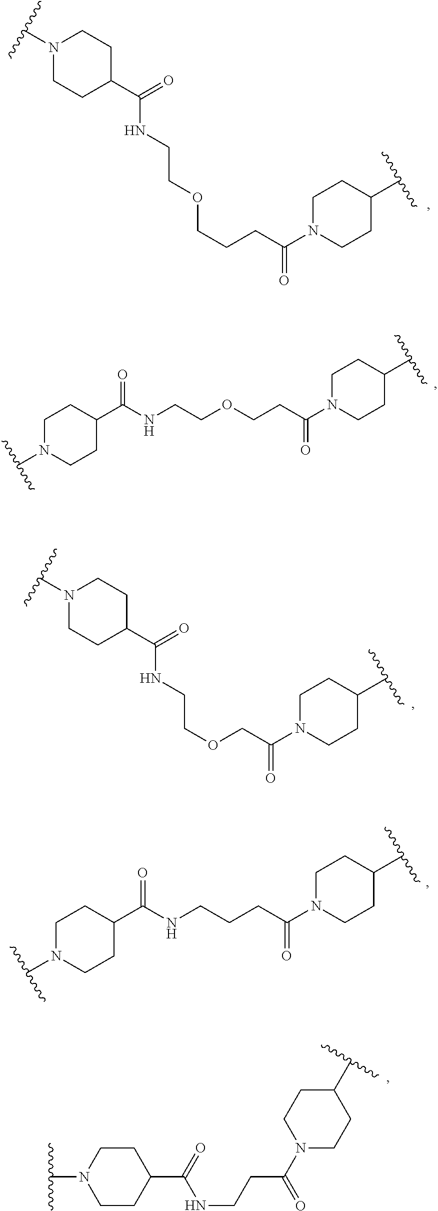 [SMILES: *C1CCN(C(=O)CCCOCCNC(=O)C2CCN(*)CC2)CC1 ; *C1CCN(C(=O)CCOCCNC(=O)C2CCN(*)CC2)CC1 ; *C1CCN(C(=O)COCCNC(=O)C2CCN(*)CC2)CC1 ; *C1CCN(C(=O)CCCNC(=O)C2CCN(*)CC2)CC1 ; *C1CCN(C(=O)CCNC(=O)C2CCN(*)CC2)CC1]

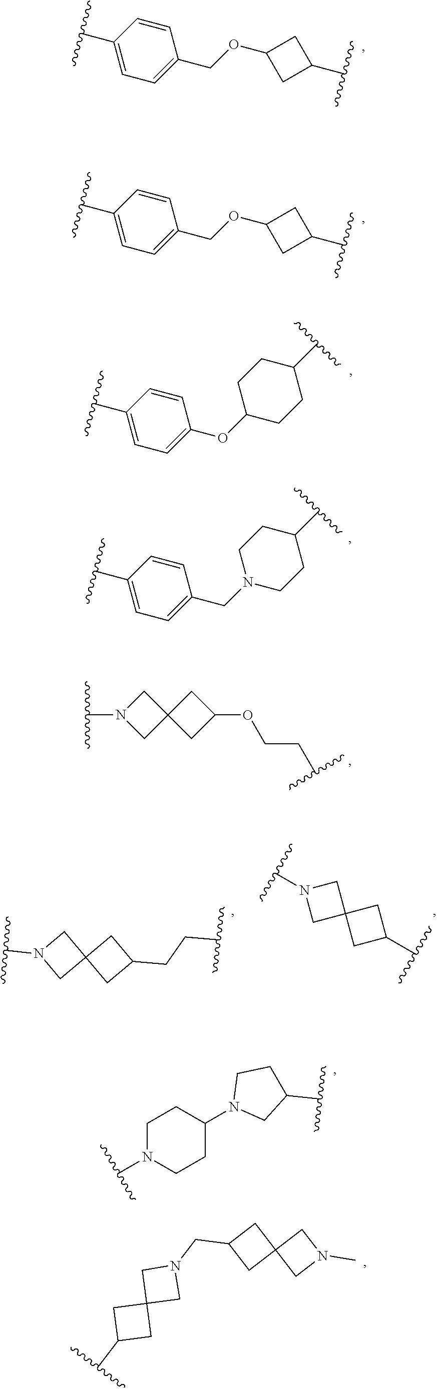

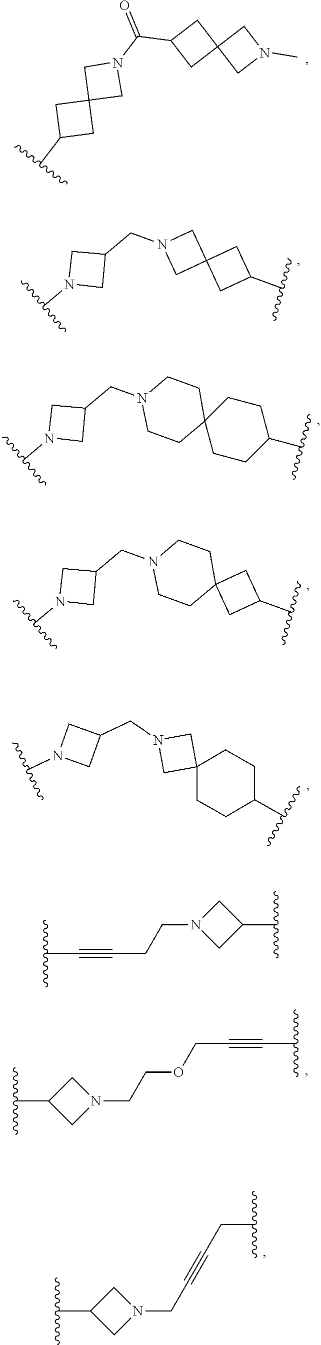

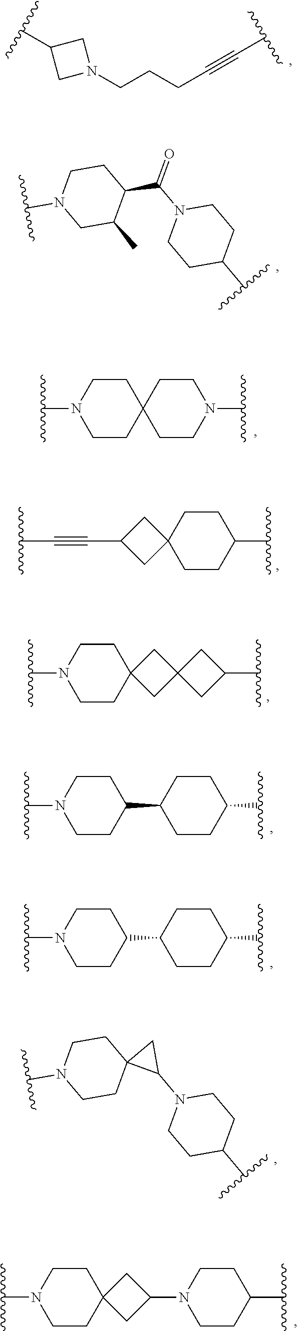

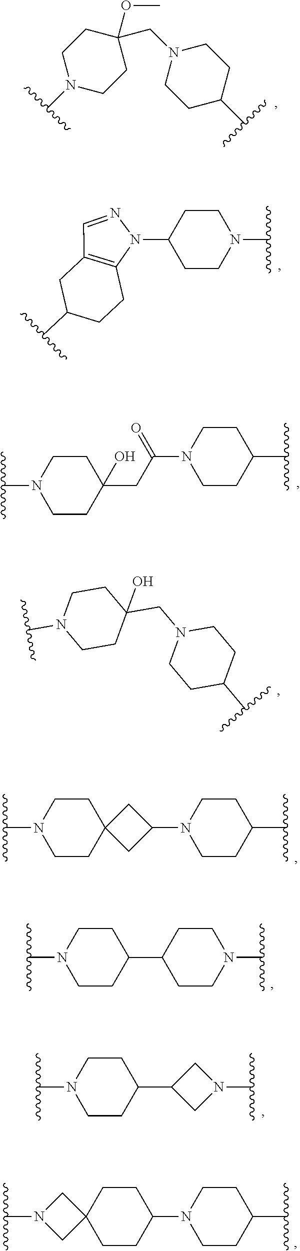

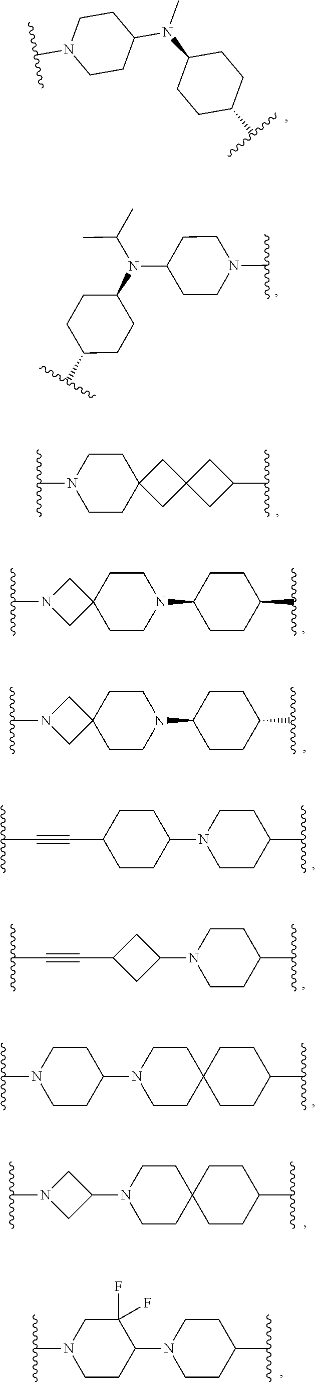

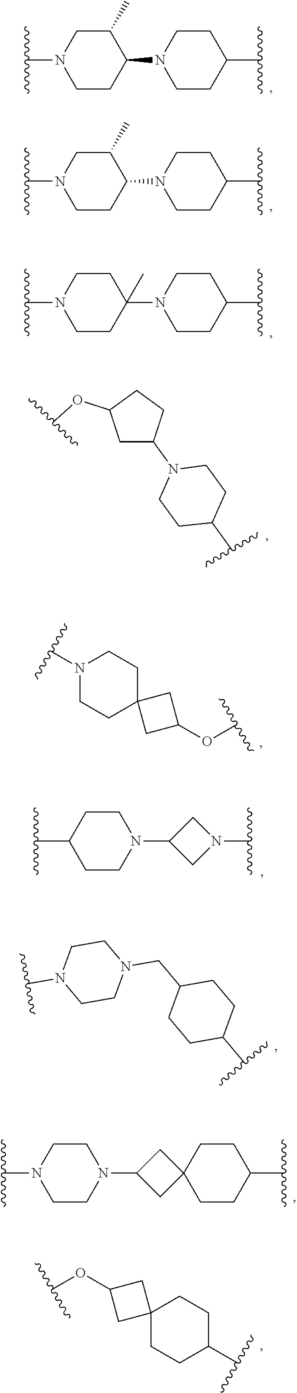

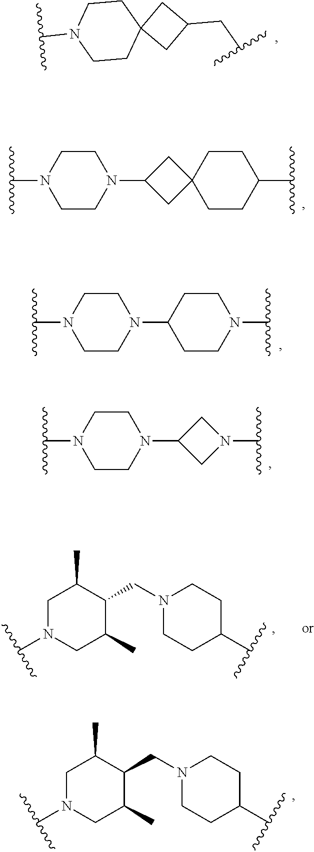 [SMILES: *CC1CC2(CCN(*)CC2)C1 ; *C1CCC2(CC1)CC(N1CCN(*)CC1)C2 ; *N1CCC(N2CCN(*)CC2)CC1 ; *N1CCN(C2CN(*)C2)CC1 ; *C1CCN(C[C@H]2[C@H](C)CN(*)C[C@@H]2C)CC1 ; *C1CCN(C[C@@H]2[C@H](C)CN(*)C[C@@H]2C)CC1]

or a pharmaceutically acceptable salt or solvate thereof.

In some embodiments, SB-CBP/p300 has the structure of Formula (IIIb), or a pharmaceutically acceptable salt or solvate thereof:

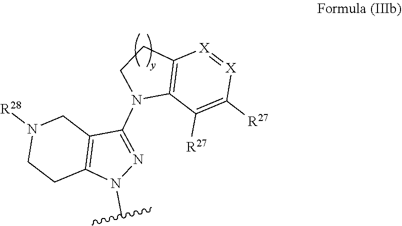

wherein:
each X is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, substituted or unsubstituted C₁-C₆cycloalkyl, or substituted or unsubstituted 3- to 8-membered heterocycloalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{a}, —S(═O)₂R^{b}, —S(═O)₂OR^{a}, —S(═O)₂N(R^{b})₂, —N(R^{b})₂, —NR^{b} C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIb), or a pharmaceutically acceptable salt or solvate thereof, wherein:
each X is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIc), or a pharmaceutically acceptable salt or solvate thereof:

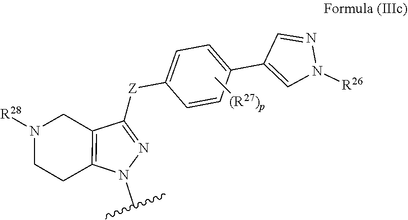

wherein:
R⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{a}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
Z is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIc), or a pharmaceutically acceptable salt or solvate thereof, wherein:
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃—C₈ cycloalkyl, or substituted or unsubstituted 3- to 8-membered heterocycloalkyl; each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIId-1) or (IIId-2), or a pharmaceutically acceptable salt or solvate thereof:

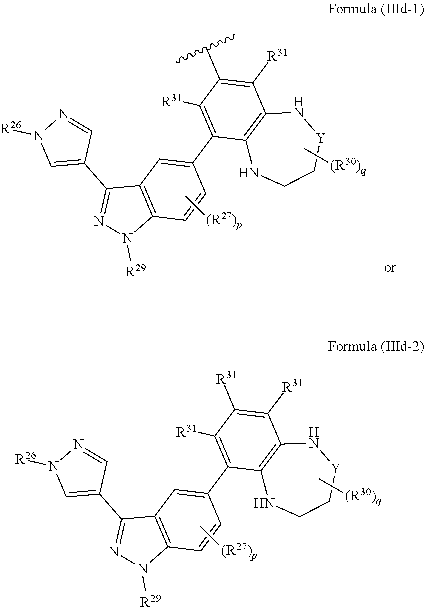

wherein:
R⁶ is hydrogen, substituted or unsubstituted C₁-C₆alkyl, substituted or unsubstituted C₁-C₆heteroalkyl, substituted or unsubstituted C₃-C₈ cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R¹⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
q is 0, 1, 2, 3, or 4;
each R¹¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
Y is —C(R¹⁰)₂- or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIId-1) or (IIId-2), or a pharmaceutically acceptable salt or solvate thereof, wherein:
R²⁶ is hydrogen, substituted or unsubstituted C₁-C₆alkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂- or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; or two R^{b} on the same N atom are taken together with the N atom to which they are attached to form a substituted or unsubstituted N-containing heterocycle.

In some embodiments, SB-CBP/p300 has the structure of Formula (IIIe), or a pharmaceutically acceptable salt or solvate thereof:

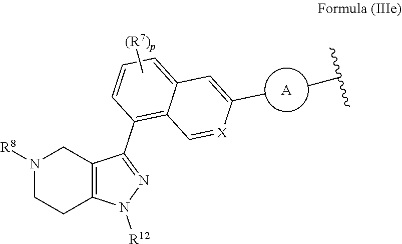

wherein:
X is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R¹² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl; Ring A is absent or substituted or unsubstituted 3- to 8-membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIe), or a pharmaceutically acceptable salt or solvate thereof, wherein:
X is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Ring A is absent or substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has one of the following structures:

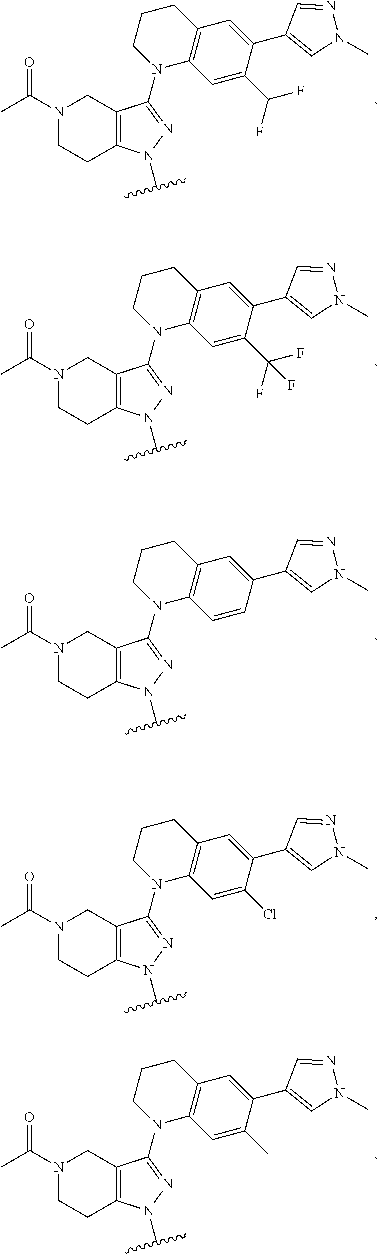

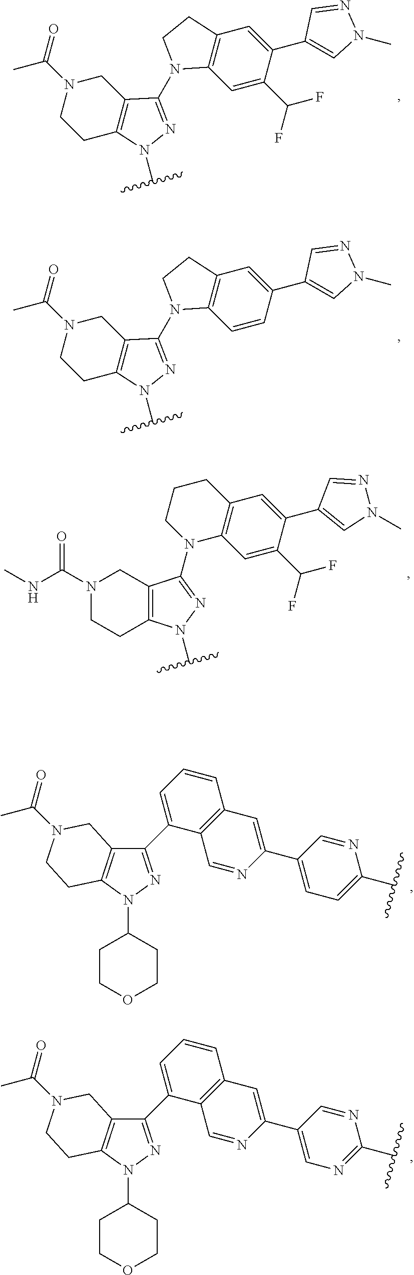

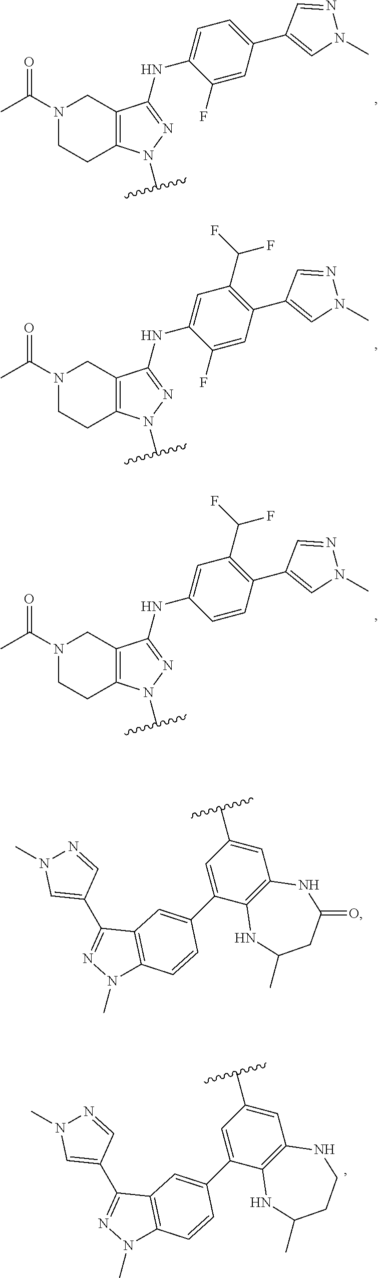

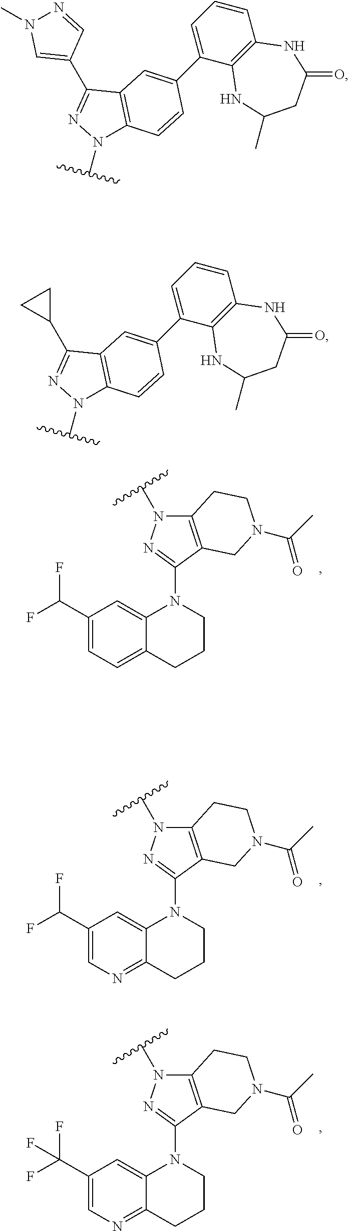 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2]

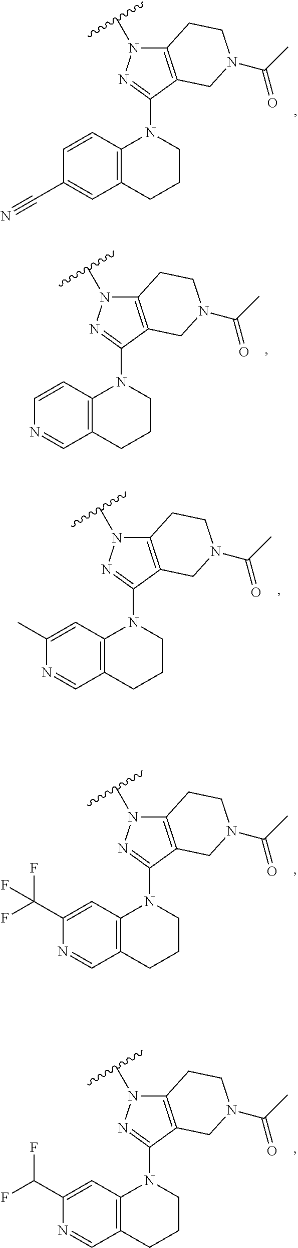

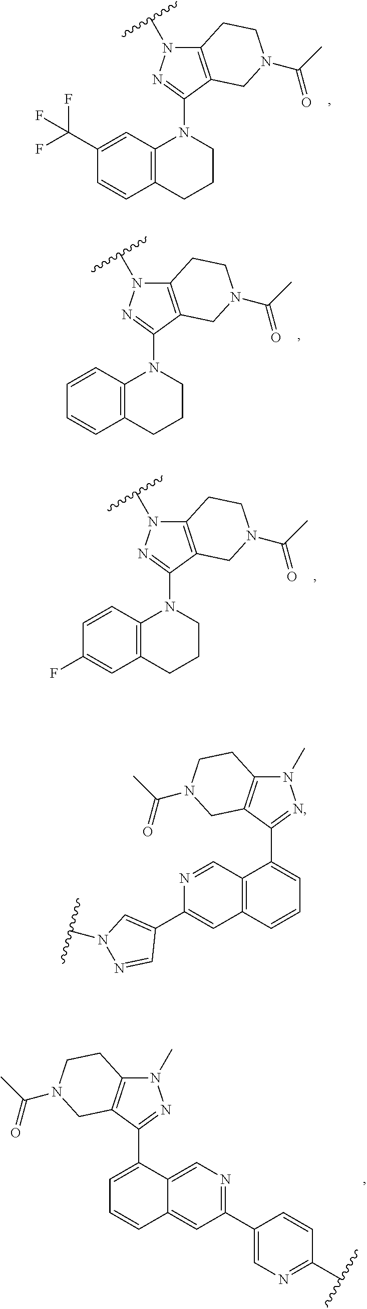

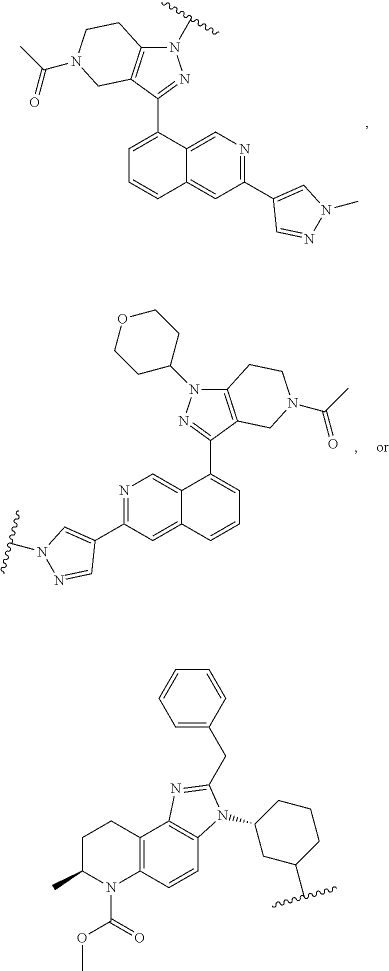

or a pharmaceutically acceptable salt or solvate thereof.

In some embodiments, SB-CBP/p300 has one of the following structures:

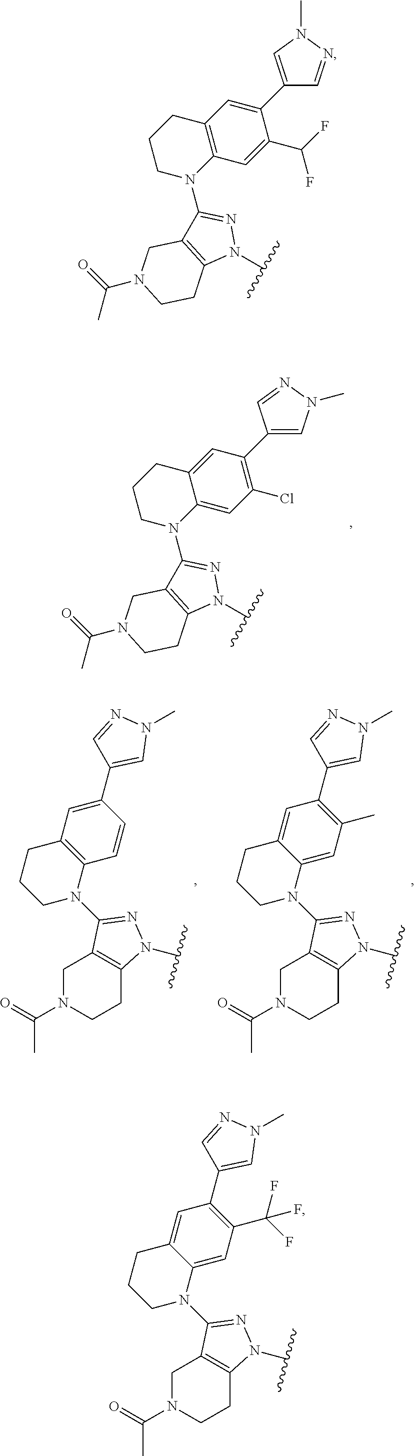 [SMILES: *n1nc(N2CCCc3cc(-c4cnn(C)c4)c(C(F)F)cc32)c2c1CCN(C(C)=O)C2 ; *n1nc(N2CCCc3cc(-c4cnn(C)c4)c(Cl)cc32)c2c1CCN(C(C)=O)C2 ; *n1nc(N2CCCc3cc(-c4cnn(C)c4)ccc32)c2c1CCN(C(C)=O)C2 ; *n1nc(N2CCCc3cc(-c4cnn(C)c4)c(C)cc32)c2c1CCN(C(C)=O)C2 ; *n1nc(N2CCCc3cc(-c4cnn(C)c4)c(C(F)(F)F)cc32)c2c1CCN(C(C)=O)C2]

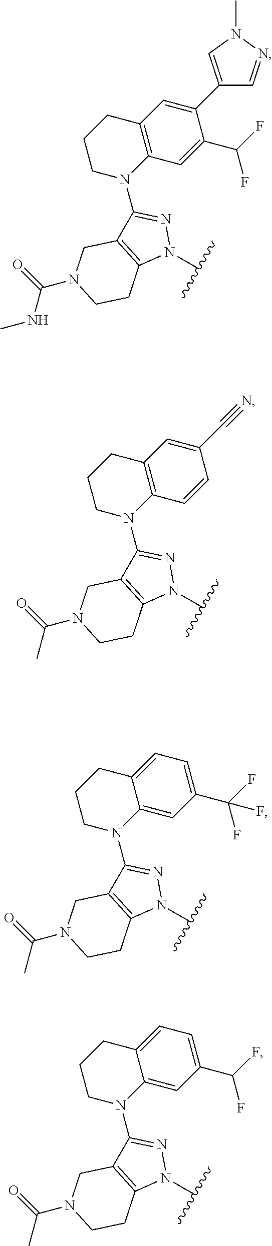 [SMILES: *n1nc(N2CCCc3cc(-c4cnn(C)c4)c(C(F)F)cc32)c2c1CCN(C(=O)NC)C2 ; *n1nc(N2CCCc3cc(C#N)ccc32)c2c1CCN(C(C)=O)C2 ; *n1nc(N2CCCc3ccc(C(F)(F)F)cc32)c2c1CCN(C(C)=O)C2 ; *n1nc(N2CCCc3ccc(C(F)F)cc32)c2c1CCN(C(C)=O)C2]

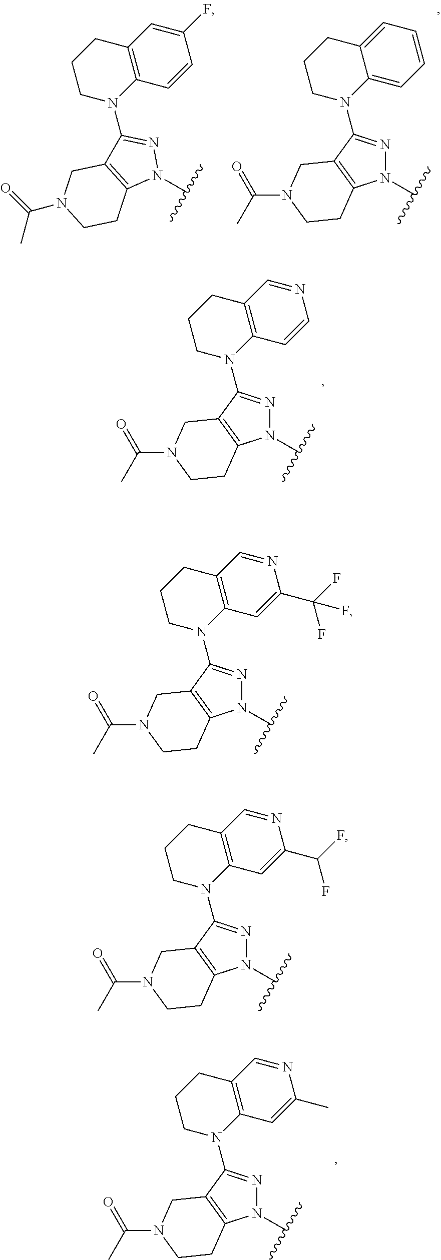 [SMILES: *n1nc(N2CCCc3cc(F)ccc32)c2c1CCN(C(C)=O)C2 ; *n1nc(N2CCCc3ccccc32)c2c1CCN(C(C)=O)C2 ; *n1nc(N2CCCc3cnccc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3cnc(C)cc32)c2c1CCN(C(C)=O)C2]

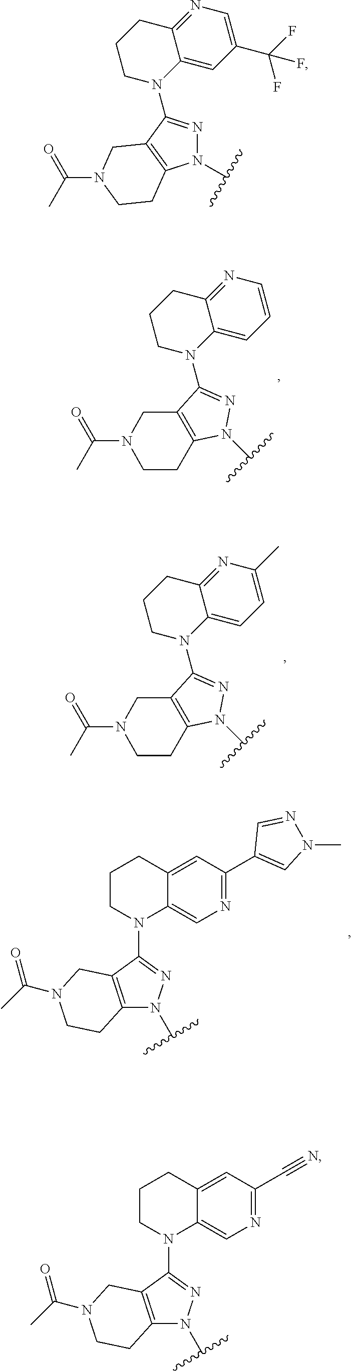

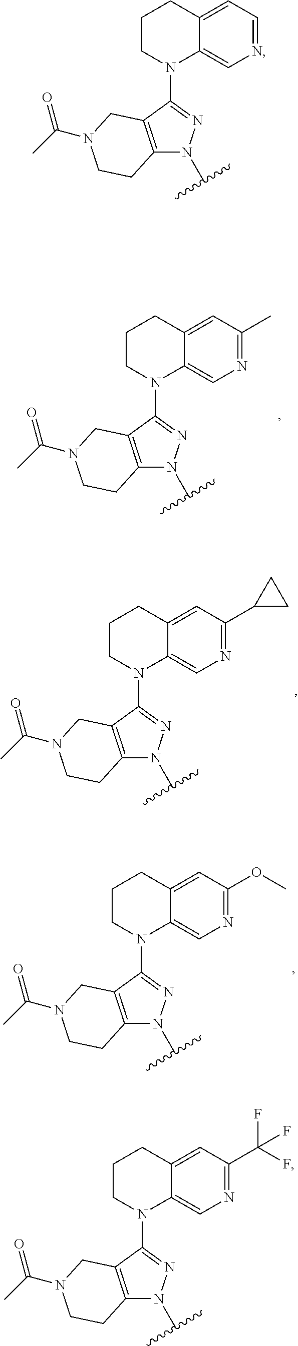

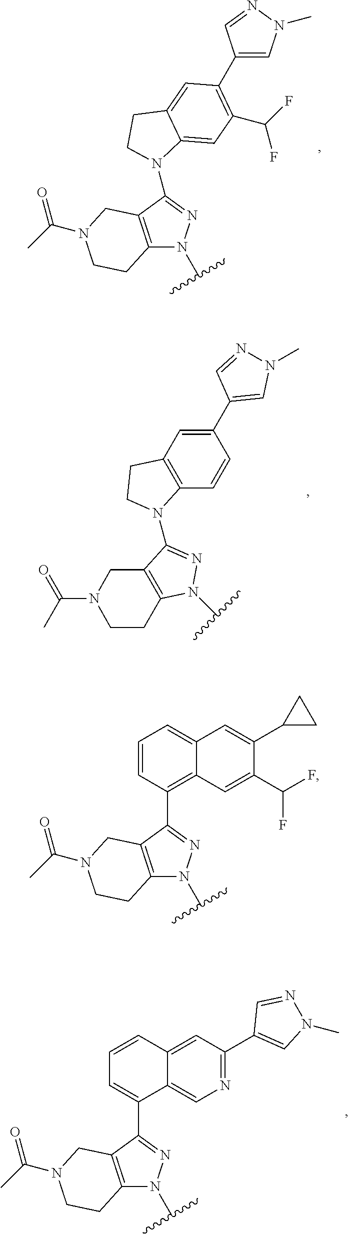

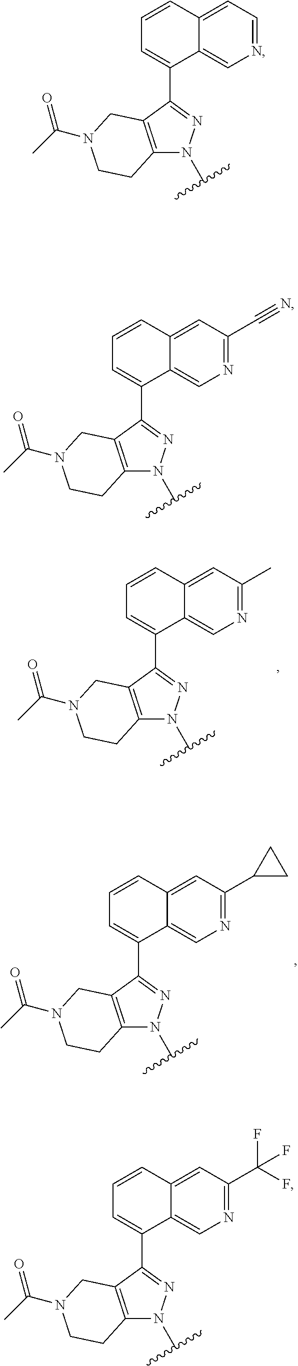

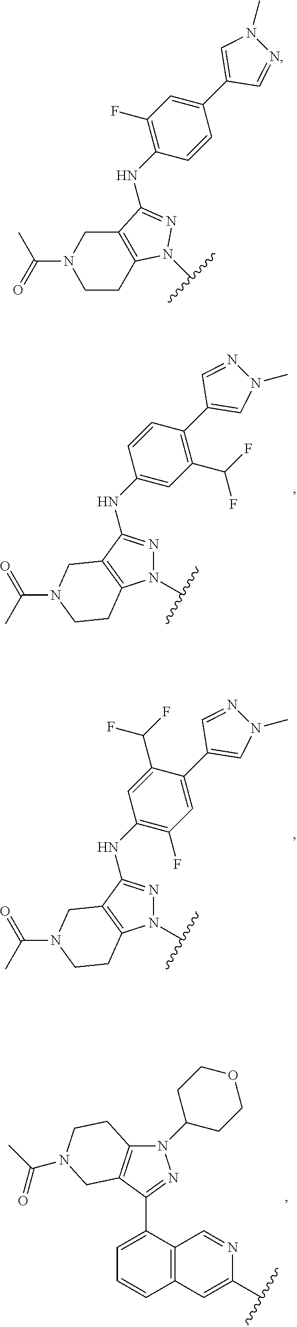

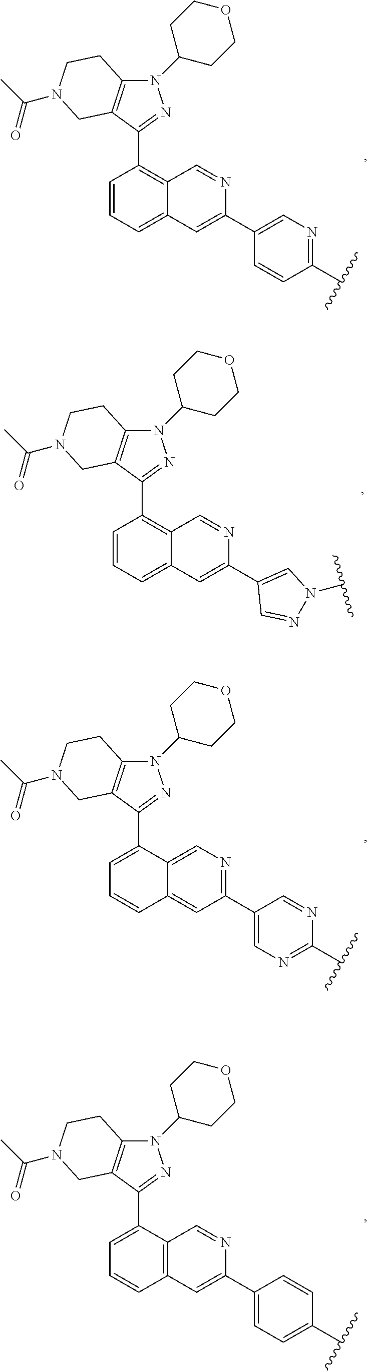 [SMILES: *c1ccc(-c2cc3cccc(-c4nn(C5CCOCC5)c5c4CN(C(C)=O)CC5)c3cn2)cn1 ; *n1cc(-c2cc3cccc(-c4nn(C5CCOCC5)c5c4CN(C(C)=O)CC5)c3cn2)cn1 ; *c1ncc(-c2cc3cccc(-c4nn(C5CCOCC5)c5c4CN(C(C)=O)CC5)c3cn2)cn1 ; *c1ccc(-c2cc3cccc(-c4nn(C5CCOCC5)c5c4CN(C(C)=O)CC5)c3cn2)cc1]

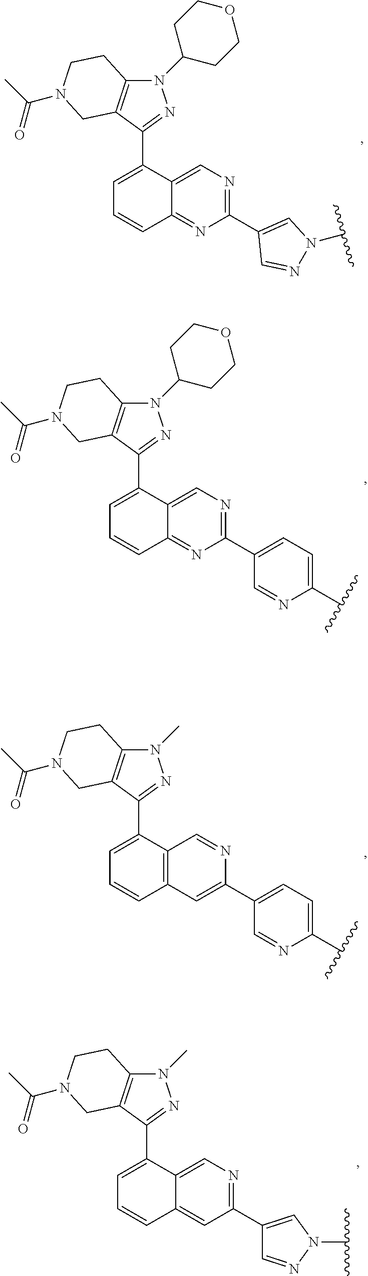 [SMILES: *n1cc(-c2ncc3c(-c4nn(C5CCOCC5)c5c4CN(C(C)=O)CC5)cccc3n2)cn1 ; *c1ccc(-c2ncc3c(-c4nn(C5CCOCC5)c5c4CN(C(C)=O)CC5)cccc3n2)cn1 ; *c1ccc(-c2cc3cccc(-c4nn(C)c5c4CN(C(C)=O)CC5)c3cn2)cn1 ; *n1cc(-c2cc3cccc(-c4nn(C)c5c4CN(C(C)=O)CC5)c3cn2)cn1]

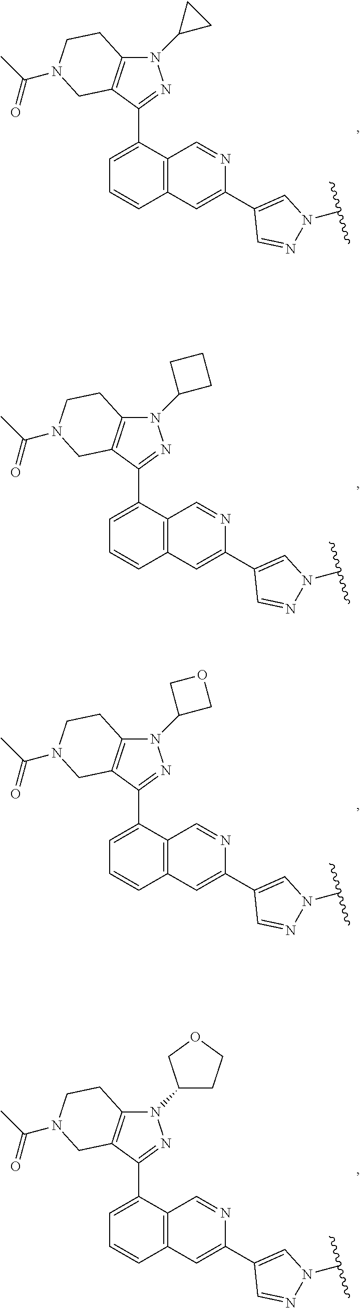 [SMILES: *n1cc(-c2cc3cccc(-c4nn(C5CC5)c5c4CN(C(C)=O)CC5)c3cn2)cn1 ; *n1cc(-c2cc3cccc(-c4nn(C5CCC5)c5c4CN(C(C)=O)CC5)c3cn2)cn1 ; *n1cc(-c2cc3cccc(-c4nn(C5COC5)c5c4CN(C(C)=O)CC5)c3cn2)cn1 ; *n1cc(-c2cc3cccc(-c4nn([C@H]5CCOC5)c5c4CN(C(C)=O)CC5)c3cn2)cn1]

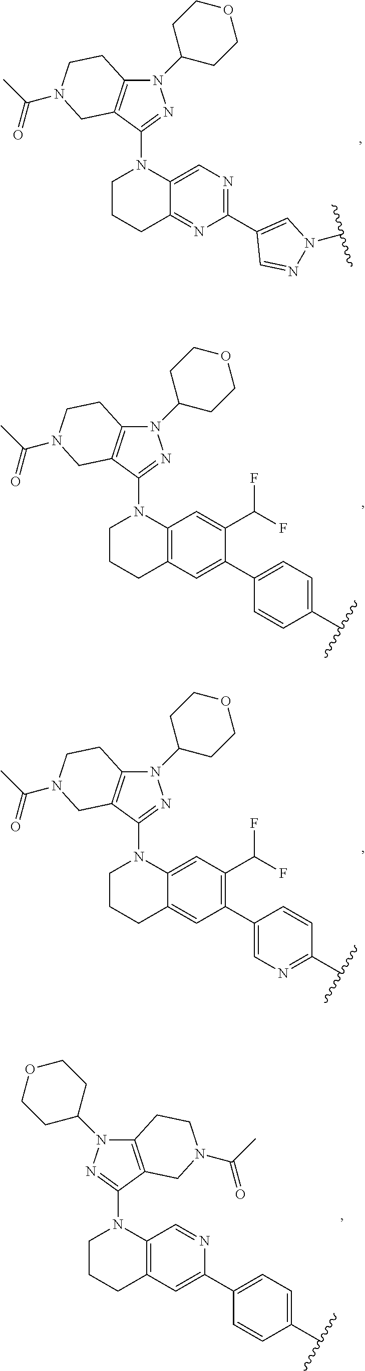 [SMILES: *n1cc(-c2ncc3c(n2)CCCN3c2nn(C3CCOCC3)c3c2CN(C(C)=O)CC3)cn1 ; *c1ccc(-c2cc3c(cc2C(F)F)N(c2nn(C4CCOCC4)c4c2CN(C(C)=O)CC4)CCC3)cc1 ; *c1ccc(-c2cc3c(cc2C(F)F)N(c2nn(C4CCOCC4)c4c2CN(C(C)=O)CC4)CCC3)cn1 ; *c1ccc(-c2cc3c(cn2)N(c2nn(C4CCOCC4)c4c2CN(C(C)=O)CC4)CCC3)cc1]

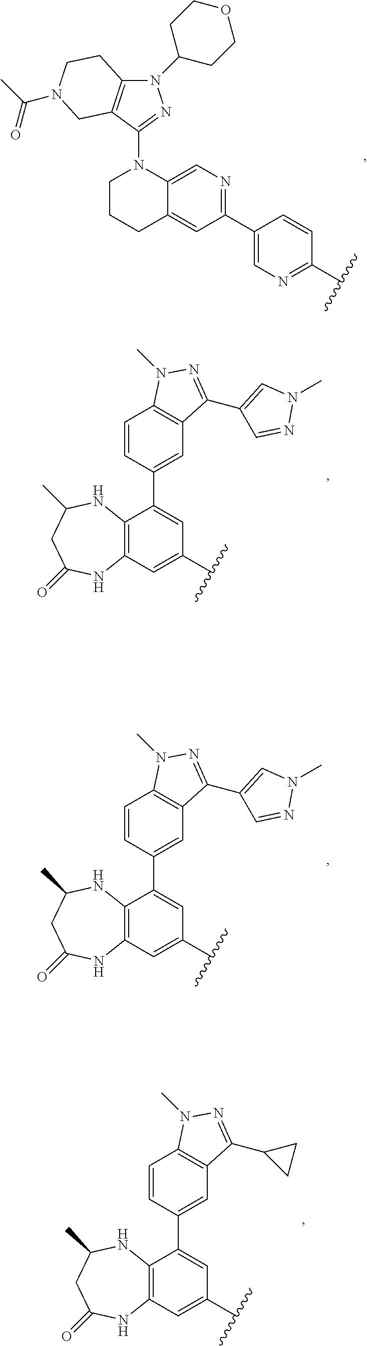 [SMILES: *c1ccc(-c2cc3c(cn2)N(c2nn(C4CCOCC4)c4c2CN(C(C)=O)CC4)CCC3)cn1 ; *c1cc2c(c(-c3ccc4c(c3)c(-c3cnn(C)c3)nn4C)c1)NC(C)CC(=O)N2 ; *c1cc2c(c(-c3ccc4c(c3)c(-c3cnn(C)c3)nn4C)c1)N[C@H](C)CC(=O)N2 ; *c1cc2c(c(-c3ccc4c(c3)c(C3CC3)nn4C)c1)N[C@H](C)CC(=O)N2]

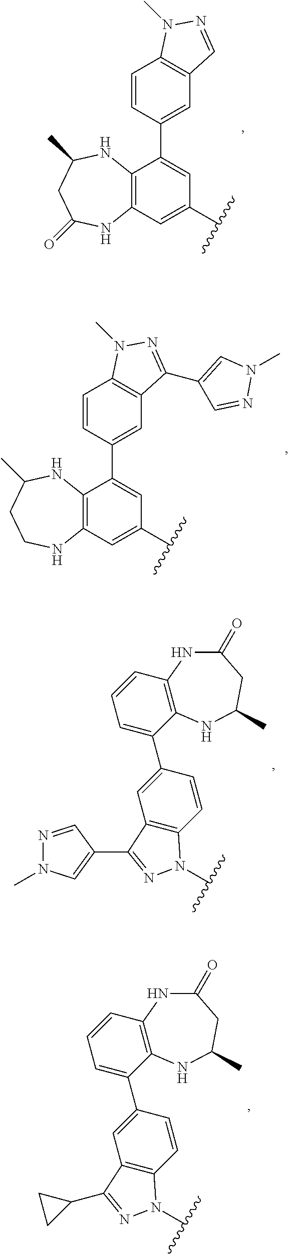

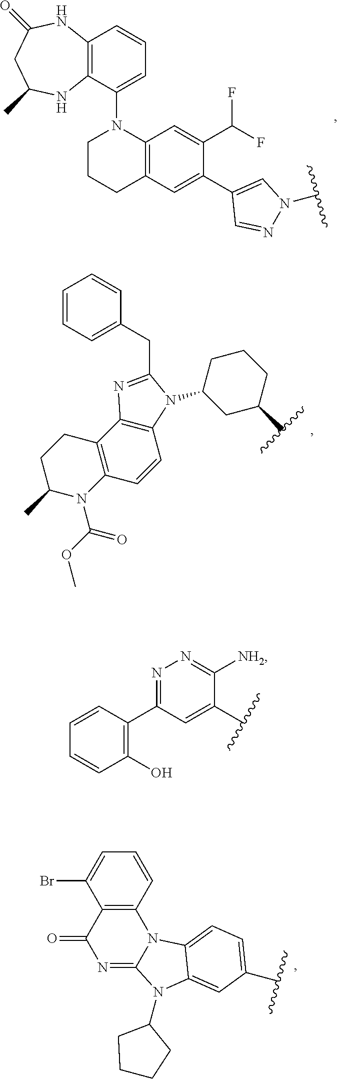 [SMILES: *n1cc(-c2cc3c(cc2C(F)F)N(c2cccc4c2N[C@@H](C)CC(=O)N4)CCC3)cn1 ; *[C@@H]1CCC[C@@H](n2c(Cc3ccccc3)nc3c4c(ccc32)N(C(=O)OC)[C@@H](C)CC4)C1 ; *c1cc(-c2ccccc2O)nnc1N ; *c1ccc2c(c1)n(C1CCCC1)c1nc(=O)c3c(Br)cccc3n21]

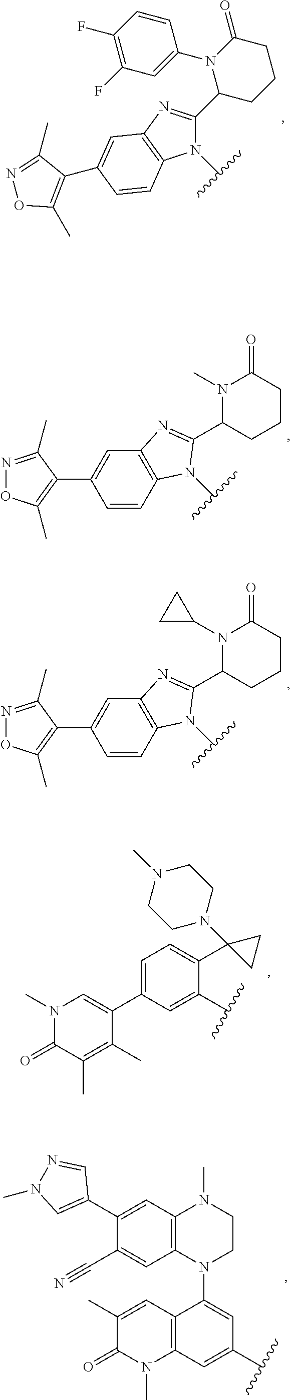 [SMILES: *n1c(C2CCCC(=O)N2c2ccc(F)c(F)c2)nc2cc(-c3c(C)noc3C)ccc21 ; *n1c(C2CCCC(=O)N2C)nc2cc(-c3c(C)noc3C)ccc21 ; *n1c(C2CCCC(=O)N2C2CC2)nc2cc(-c3c(C)noc3C)ccc21 ; *c1cc(-c2cn(C)c(=O)c(C)c2C)ccc1C1(N2CCN(C)CC2)CC1 ; *c1cc(N2CCN(C)c3cc(-c4cnn(C)c4)c(C#N)cc32)c2cc(C)c(=O)n(C)c2c1]

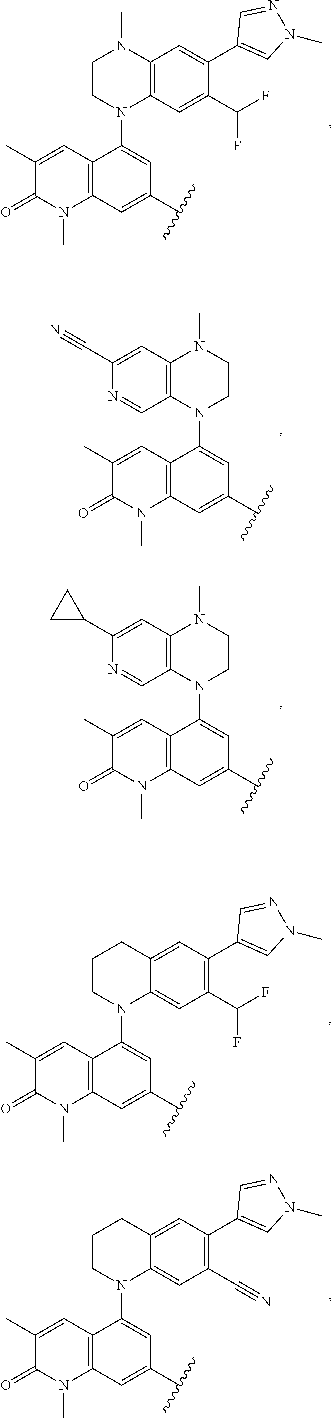 [SMILES: *c1cc(N2CCN(C)c3cc(-c4cnn(C)c4)c(C(F)F)cc32)c2cc(C)c(=O)n(C)c2c1 ; *c1cc(N2CCN(C)c3cc(C#N)ncc32)c2cc(C)c(=O)n(C)c2c1 ; *c1cc(N2CCN(C)c3cc(C4CC4)ncc32)c2cc(C)c(=O)n(C)c2c1 ; *c1cc(N2CCCc3cc(-c4cnn(C)c4)c(C(F)F)cc32)c2cc(C)c(=O)n(C)c2c1 ; *c1cc(N2CCCc3cc(-c4cnn(C)c4)c(C#N)cc32)c2cc(C)c(=O)n(C)c2c1]

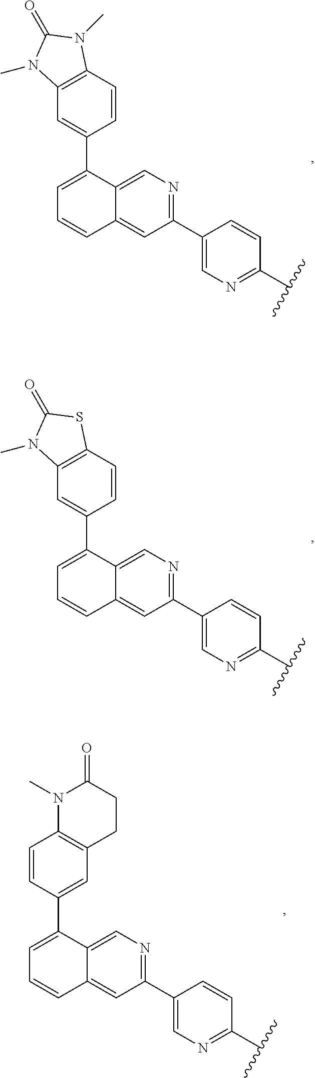 [SMILES: *c1ccc(-c2cc3cccc(-c4ccc5c(c4)n(C)c(=O)n5C)c3cn2)cn1 ; *c1ccc(-c2cc3cccc(-c4ccc5sc(=O)n(C)c5c4)c3cn2)cn1 ; *c1ccc(-c2cc3cccc(-c4ccc5c(c4)CCC(=O)N5C)c3cn2)cn1]

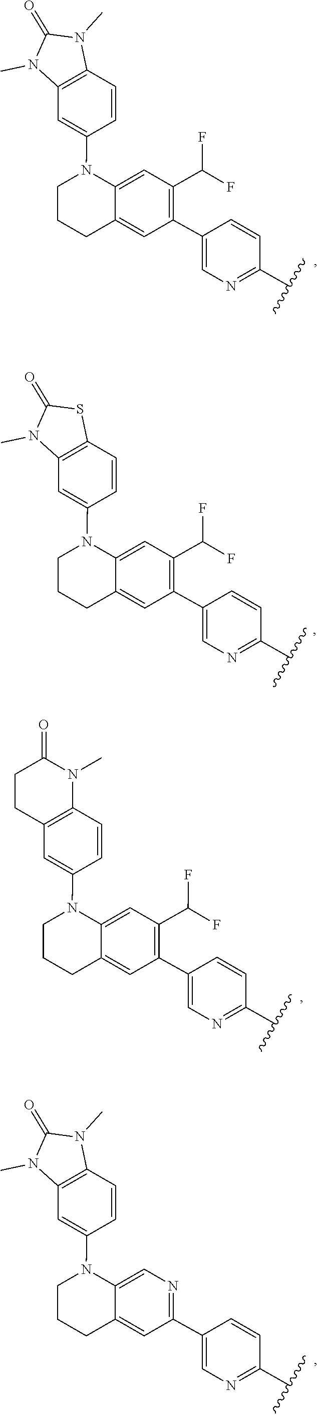 [SMILES: *c1ccc(-c2cc3c(cc2C(F)F)N(c2ccc4c(c2)n(C)c(=O)n4C)CCC3)cn1 ; *c1ccc(-c2cc3c(cc2C(F)F)N(c2ccc4sc(=O)n(C)c4c2)CCC3)cn1 ; *c1ccc(-c2cc3c(cc2C(F)F)N(c2ccc4c(c2)CCC(=O)N4C)CCC3)cn1 ; *c1ccc(-c2cc3c(cn2)N(c2ccc4c(c2)n(C)c(=O)n4C)CCC3)cn1]

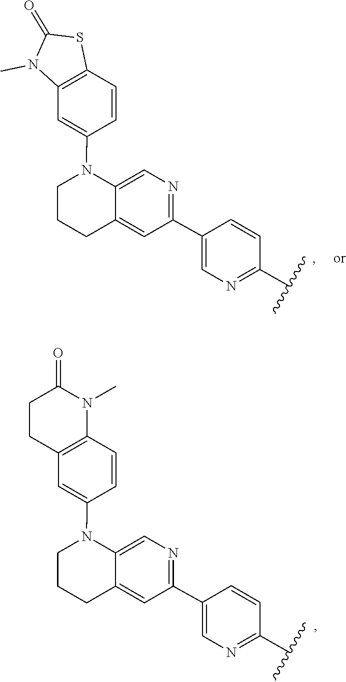 [SMILES: *c1ccc(-c2cc3c(cn2)N(c2ccc4sc(=O)n(C)c4c2)CCC3)cn1 ; *c1ccc(-c2cc3c(cn2)N(c2ccc4c(c2)CCC(=O)N4C)CCC3)cn1]

or a pharmaceutically acceptable salt or solvate thereof.

In some embodiments, SB-CBP/p300 has one of the following structures:

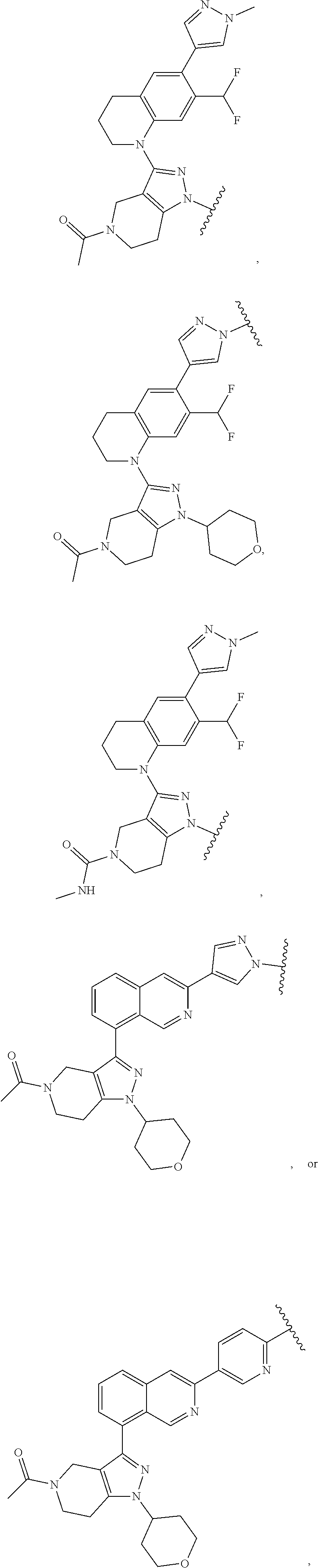

or a pharmaceutically acceptable salt or solvate thereof.

Embodiment 1. A heterobifunctional conditional inhibitor compound of Formula (I), or a pharmaceutically acceptable salt or solvate thereof:

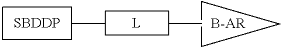

wherein:
SBDDP is a silent binder of a disease-dependent protein (DDP);
L is an optional linker; wherein L is covalently attached at a position of SBDDP that is solvent exposed when SBDDP binds to the DDP;
B-AR is a binder of the androgen receptor (AR), wherein B-AR comprises:
1) a head group that occupies the ligand-binding domain (LBD) of AR and is covalently attached to a core moiety; and
2) an optional tail moiety covalently attached to the core moiety; wherein the core comprises an optionally substituted cyclobutyl having the structure of Formula (C):

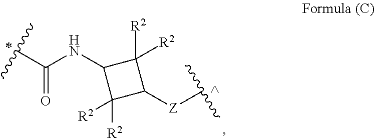

the head group is covalently attached to Z at position (A);
the optional tail moiety comprises a ring D that is covalently attached to the amide (*), wherein ring D is a 5-, 6-, 8-, 9- or 10-membered aryl or a 5-, 6-, 8-, 9- or 10-membered heteroaryl that is optionally substituted with s R³.
wherein:
Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈ cycloalkyl or a substituted or unsubstituted monocyclic 3- to 8-membered heterocycloalkyl;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle; and
each s is 1, 2, or 3;
wherein L is covalently attached to the core moiety at position (*) if the optional tail moiety is absent, or L is covalently attached to the tail moiety if present;
wherein DDP and AR are both expressed in a cell of interest (COI), and wherein the relative abundance of the AR in the COI is greater than the relative abundance of the DDP in the COI; or the COI is a diseased cell, and the AR is overexpressed, overactive, or both overexpressed and overactive, or amplified in the diseased cell as compared to when the COI is a non-diseased cell.

Embodiment 2. The compound of embodiment 1, or a pharmaceutically acceptable salt or solvate thereof, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

Embodiment 3. The compound of embodiment 1 or embodiment 2, or a pharmaceutically acceptable salt or solvate thereof, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

Embodiment 4. The compound of any one of embodiments 1-3, or a pharmaceutically acceptable salt or solvate thereof, wherein DDP is CREB-binding protein (CBP)/p300.

Embodiment 5. The compound of embodiment 4, or a pharmaceutically acceptable salt or solvate thereof, wherein SBDDP binds in the acetyl-lysine (KAc) binding site of the bromodomain of CBP/p300.

Embodiment 6. The compound of embodiment 4, or a pharmaceutically acceptable salt or solvate thereof, wherein the SBDDP comprises an acetyl-lysine mimetic moiety that binds in the acetyl-lysine (KAc) binding site of the bromodomain of CBP/p300.

Embodiment 7. The compound of embodiment 6, or a pharmaceutically acceptable salt or solvate thereof, wherein:
the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300; and the head group of B-AR forms hydrogen bonds with the side chains of Gln 711 and Arg752 of the LBD of AR.

Embodiment 8. The compound of any one of embodiments 1-7, or a pharmaceutically acceptable salt or solvate thereof, wherein: SBDDP further comprises a moiety that interacts with Arg1173 in the bromodomain of CBP or Asn1137 in the bromodomain of p300.

Embodiment 9. The compound of any one of embodiments 1-8, or a pharmaceutically acceptable salt or solvate thereof, wherein SBDDP further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2).

Embodiment 10. The compound of any one of embodiments 6-9, or a pharmaceutically acceptable salt or solvate thereof, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

Embodiment 11. The compound of embodiment 9 or 10, or a pharmaceutically acceptable salt or solvate thereof, wherein L is covalently attached to SBDDP on:
the acetyl-lysine mimetic moiety; or
the moiety that occupies the lipophilic shelf (LPF) region of the bromodomain of CBP/p300, if present; or
the moiety that occupies the BC Loop region of the bromodomain of CBP/p300, if present; wherein L is covalently attached to SBDDP at a position that does not interfere with the binding of the
acetyl-lysine mimetic moiety in the acetylated lysine (KAc) binding site of CBP/p300.

Embodiment 12. The compound of any one of embodiments 1-11, or a pharmaceutically acceptable salt or solvate thereof, wherein:
the head group of B-AR comprises 4-cyanophenyl; 3-fluoro-4-cyanophenyl; 3-chloro-4-cyanophenyl; 3-methoxy-4-cyanophenyl; 3-methyl-4-cyanophenyl; 3-trifluroromethyl-4-cyanophenyl; 3-trifluroromethoxy-4-cyanophenyl; 5-fluoro-6-cyanopyridin-3-yl; 5-chloro-6-cyanopyridin-3-yl; 5-methoxy-6-cyanopyridin-3-yl; 5-methyl-6-cyanopyridin-3-yl; 5-trifluroromethyl-6-cyanopyridin-3-yl; 5-trifluroromethoxy-6-cyanopyridin-3-yl; [1,2,4]triazolo[4,3-b]pyridazin-6-yl; or 3-(trifluoromethyl)-[1,2,4]triazolo[4,3-b]pyridazin-6-yl; and
each R² is —CH₃.

Embodiment 13. The compound of any one of embodiments 1-12, or a pharmaceutically acceptable salt or solvate thereof, wherein the acetyl-lysine mimetic moiety that binds in the acetyl-lysine (KAc) binding site of the bromodomain of CBP/p300 comprises:
1-(1,4,6,7-tetrahydro-5H-pyrazolo[4,3-c]pyridin-5-yl)ethan-1-one; or N-methyl-1,4,6,7-tetrahydro-5H-pyrazolo[4,3-c]pyridine-5-carboxamide; wherein the acetyl-lysine mimetic moiety optionally further comprises:
1) a moiety at the 1-position of the 1,4,6,7-tetrahydro-5H-pyrazolo[4,3-c]pyridin-5-yl group that occupies the lipophilic shelf (LPF) region of the bromodomain of CBP/p300; or
2) a moiety that at the 3-position of the 1,4,6,7-tetrahydro-5H-pyrazolo[4,3-c]pyridin-5-yl group occupies the BC Loop region of the bromodomain of CBP/p300; or
3) both 1) and 2).

Embodiment 14. A heterobifunctional conditional inhibitor compound of Formula (II), or a pharmaceutically acceptable salt or solvate thereof:

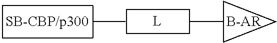

wherein:
SB-CBP/p300 is a silent binder of human CREB-binding protein (CBP) or human E1A-binding protein p300 (p300) (CBP/p300);
L is an optional linker; wherein L is covalently attached at a position of SB-CBP/p300 that is solvent exposed when SB binds to CBP/p300;
B-AR is a binder of the androgen receptor (AR), wherein the B-AR comprises:
1) a head group that occupies the ligand-binding domain (LBD) of AR and is covalently attached to a core moiety; and
2) an optional tail moiety covalently attached to the core moiety; wherein the core comprises an optionally substituted cyclobutyl having the structure of Formula (C):

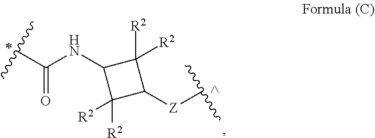

wherein:
the head group is covalently attached to Z at position (\hat{ });
the optional tail moiety comprises a ring D that is covalently attached to the amide (*), wherein ring D is a 5-, 6-, 8-, 9- or 10-membered aryl or a 5-, 6-, 8-, 9- or 10-membered heteroaryl that is optionally substituted with s R³.
Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈cycloalkyl or a substituted or unsubstituted monocyclic 3- to 8-membered heterocycloalkyl;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle; and
each s is 1, 2, or 3;
wherein L is covalently attached to the core moiety at position (*) if the optional tail moiety is absent, or L is covalently attached to the tail moiety if present.

Embodiment 15. The compound of any one of embodiments 1-11 or 14, or a pharmaceutically acceptable salt or solvate thereof, wherein:
the head group of B-AR forms hydrogen bonds with the side chains of Gln 711 and Arg752 of the LBD of AR; and each R² is independently hydrogen, —CH₃, —CD₃, —CH₂F, —CHF₂, or —CF₃.

Embodiment 16. The compound of any one of embodiments 1-11 or 14, or a pharmaceutically acceptable salt or solvate thereof, wherein: each R² is —CH₃.

Embodiment 17. The compound of any one of embodiments 1-11 or 14, or a pharmaceutically acceptable salt or solvate thereof, wherein B-AR comprises a head group and an optional core, wherein the head group is selected from:

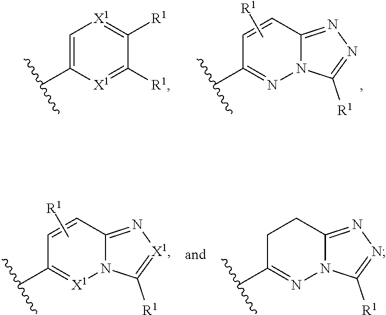

each X¹ is independently —CR¹— or —N—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂.

Embodiment 18. The compound of any one of embodiments 1-11 or 14, or a pharmaceutically acceptable salt or solvate thereof, wherein B-AR comprises a head group and an optional core, wherein the head group is selected from:

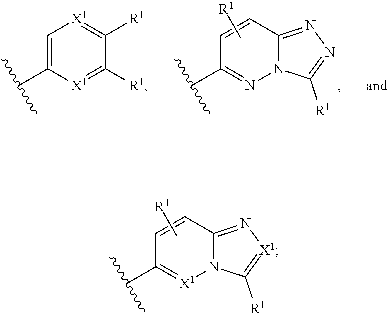

one X¹ is —CR¹— and the other X¹ is —CR¹— or —N—;
each R¹ is independently hydrogen, F, Cl, Br, I, —CH₃, —CD₃, —CH₂CH₃, —CH₂F, —CHF₂, —CF₃, —CH₂CH₂F, —CH₂CHF₂, —CH₂CF₃, —OH, —OCF₃, —OCH₃, —OCD₃, —OCH₂CH₃, —CN, —C(═O)NH₂, —C(═O)NH(CH₃), or —C(═O)NH(CD₃).

Embodiment 19. The compound of any one of embodiments 1-11 or 14, or a pharmaceutically acceptable salt or solvate thereof, wherein B-AR comprises a head group and an optional core, wherein the head group is selected from:

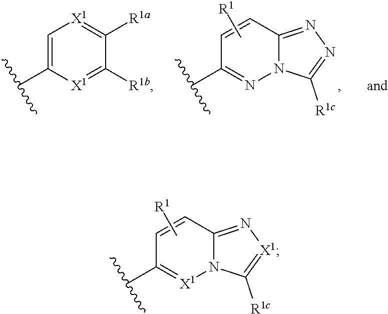

each X¹ is independently —CR¹— or —N—;
R^{1a} is —CN, —NO₂, —C(═O)R⁵, —C(═O)OR⁵, or —C(═O)N(R⁵)₂;
R^{1b} is hydrogen, halogen, C₁-C₄alkyl, C₁-C₄fluoroalkyl, —CN, —OH, —OR⁴, or —SR⁴;
R^{1c} is hydrogen, halogen, C₁-C₄alkyl, C₁-C₄fluoroalkyl, or —CN;
each R¹ is independently hydrogen, halogen, C₁-C₄alkyl, C₁-C₄fluoroalkyl, —CN, —OH, or —OR⁴;
each R⁴ is independently C₁-C₄alkyl, or C₁-C₄fluoroalkyl;
each R⁵ is independently hydrogen, C₁-C₄alkyl, or C₁-C₄fluoroalkyl.

Embodiment 20. The compound of any one of embodiments 1-11 or 14, or a pharmaceutically acceptable salt or solvate thereof, wherein B-AR comprises a head group and an optional core, wherein the head group is selected from:

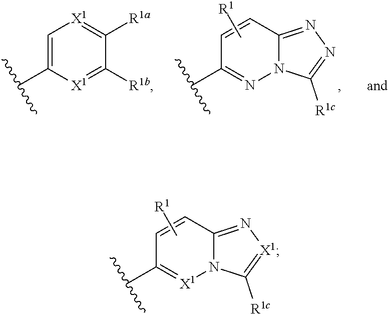

one X¹ is —CR¹— and the other X¹ is —CR¹— or —N—;
R^{1a} is —CN, —NO₂, —C(═O)NH₂ or —C(═O)NH(CH₃); R^{1b} is hydrogen, F, Cl, Br, I, —CH₃, —CH₂CH₃, —CH₂F, —CHF₂, —CF₃, —OH, —OCF₃, —OCH₃, —OCH₂CH₃, or —CN;
each R^{1c} is hydrogen, F, Cl, Br, —CH₃, —CH₂F, —CHF₂, or —CF₃;
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, or —OCH₂CH₃.

Embodiment 21. The compound of any one of embodiments 1-11 or 14, or a pharmaceutically acceptable salt or solvate thereof, wherein B-AR comprises a head group and an optional core, wherein the head group is selected from:

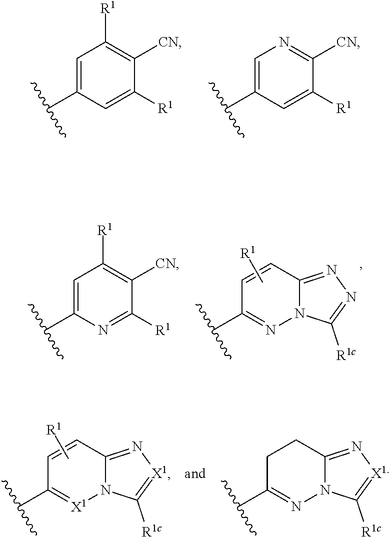

Embodiment 22. The compound of any one of embodiments 1-11 or 14, or a pharmaceutically acceptable salt or solvate thereof, wherein B-AR comprises a head group and an optional core, wherein the head group is selected from:

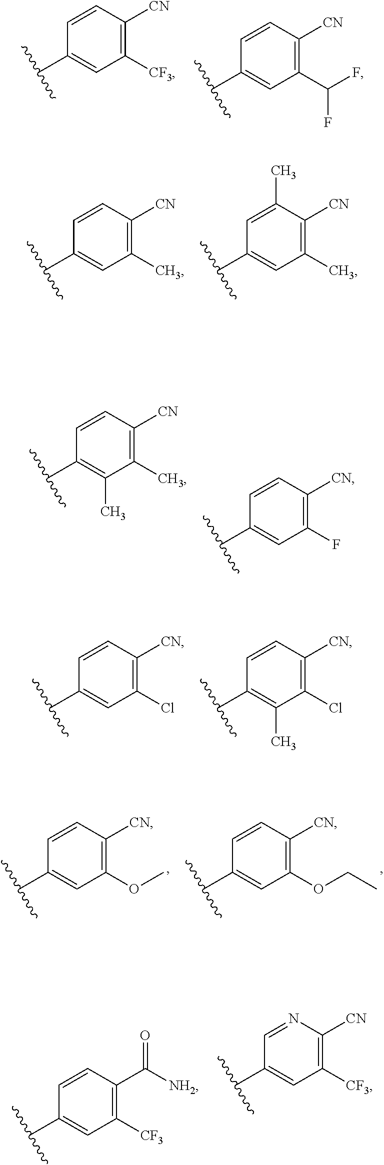

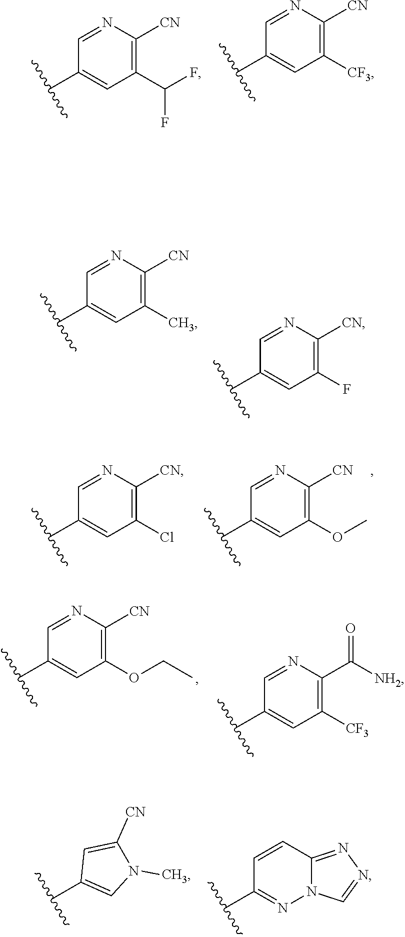

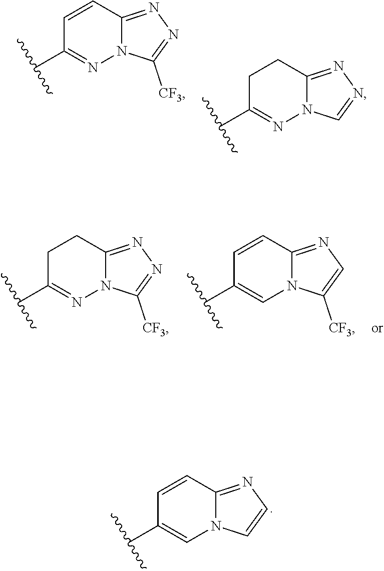 [SMILES: *c1ccc2nnc(C(F)(F)F)n2n1 ; *C1=Nn2cnnc2CC1 ; *C1=Nn2c(nnc2C(F)(F)F)CC1 ; *c1ccc2ncc(C(F)(F)F)n2c1 ; *c1ccc2nccn2c1]

Embodiment 23. The compound of any one of embodiments 1-11 or 14-20, or a pharmaceutically acceptable salt or solvate thereof, wherein B-AR has the structure of Formula (IIa), or a pharmaceutically acceptable salt or solvate thereof:

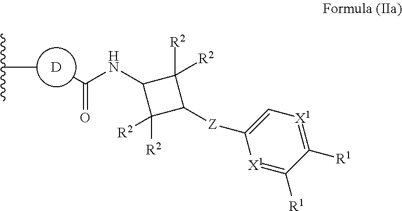

wherein:
each X¹ is independently —CR¹— or —N—;
Z is —O— or —NR⁵—;
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂ or —C(═O)NH(CH₃);
each R² is C₁-C₆alkyl;
ring D that is a 5-, 6-, 8-, 9- or 10-membered aryl or a 5-, 6-, 8-, 9- or 10-membered heteroaryl that is optionally substituted with s R³.
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂;
s is 1, 2, or 3.

Embodiment 24. The compound of embodiment 23, or a pharmaceutically acceptable salt or solvate thereof, or a pharmaceutically acceptable salt or solvate thereof:
one X¹ is —CR¹— and the other X¹ is —CR¹— or —N—;
each R² is —CH₃.

Embodiment 25. The compound of any one of embodiments 1-11 or 14-20, or a pharmaceutically acceptable salt or solvate thereof, wherein B-AR has the structure of Formula (IIIa), or a pharmaceutically acceptable salt or solvate thereof:

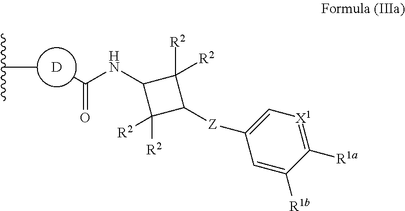

wherein:
X¹ is —CR¹— or —N—;
Z is —O— or —NR⁵—;
R^{1a} is —CN, —NO₂, —C(═O)R⁵, —C(═O)OR⁵, or —C(═O)N(R⁵)₂;
R^{1b} is hydrogen, halogen, C₁-C₄alkyl, C₁-C₄fluoroalkyl, —CN, —OH, or —OR⁴;
each R¹ is independently hydrogen, halogen, C₁-C₄alkyl, C₁-C₄fluoroalkyl, —CN, —OH, or —OR⁴;
each R² is —CH₃;
ring D that is a 5-, 6-, 8-, 9- or 10-membered aryl or a 5-, 6-, 8-, 9- or 10-membered heteroaryl that is optionally substituted with s R³;
each R³ is independently hydrogen, halogen, substituted or unsubstituted C₁-C₄alkyl, C₁-C₄fluoroalkyl, —CN, —OH, —OR⁴, or —N(R⁵)₂;
s is 1, 2, or 3;
each R⁴ is independently C₁-C₄alkyl, or C₁-C₄fluoroalkyl;
each R⁵ is independently hydrogen, C₁-C₄alkyl, or C₁-C₄fluoroalkyl.

Embodiment 26. The compound of any one of embodiments 23-25, or a pharmaceutically acceptable salt or solvate thereof, wherein ring D is phenyl, naphthyl, pyridinyl, pyrimidinyl, pyridazinyl, pyrazinyl, triazinyl, pyrrolyl, thiazolyl, oxazolyl, isoxazolyl, oxadiazolyl, imidazolyl, pyrazolyl, isothiazolyl, triazolyl, and tetrazolyl, wherein each ring D is optionally substituted with s R³.

Embodiment 27. The compound of any one of embodiments 23-25, or a pharmaceutically acceptable salt or solvate thereof, wherein ring D is

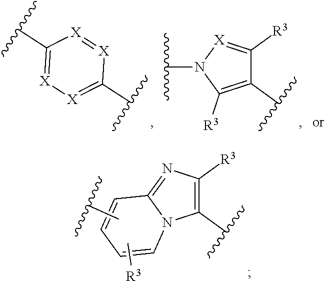

and
each X is independently —CR³— or —N—.

Embodiment 28. The compound of any one of embodiments 23-25, or a pharmaceutically acceptable salt or solvate thereof, wherein ring D is

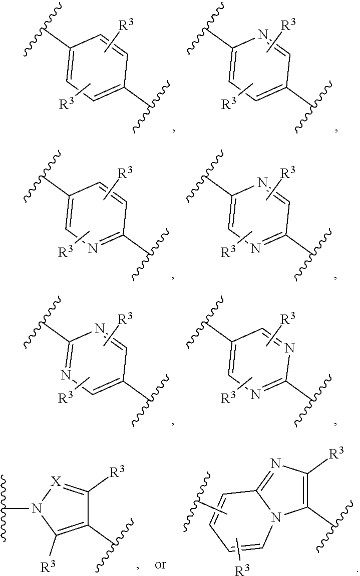

Embodiment 29. The compound of any one of embodiments 1-11 or 14-20, or a pharmaceutically acceptable salt or solvate thereof, wherein B-AR has the structure of Formula (IVa), or a pharmaceutically acceptable salt or solvate thereof:

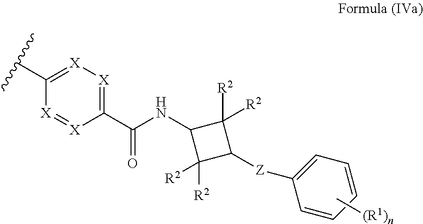

wherein:
each X is independently —CR³— or —N—;
Z is —O— or —NR⁵—;
n is 2 and one R¹ is halogen, —CH₃, —OCH₃, or —CF₃, and the other R¹ is —CN;
each R² is —CH₃.

Embodiment 30. The compound of embodiment 20, or a pharmaceutically acceptable salt or solvate thereof, wherein:
one R¹ is —Cl, —CH₃, —OCH₃, or —CF₃, and the other R¹ is —CN.

Embodiment 31. The compound of any one of embodiments 1-11 or 14-20, or a pharmaceutically acceptable salt or solvate thereof, wherein B-AR has the structure of Formula (Va), or a pharmaceutically acceptable salt or solvate thereof:

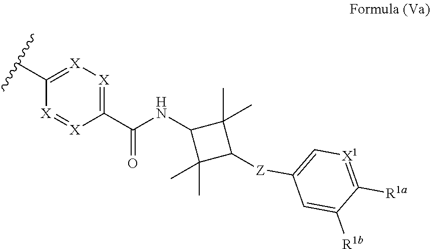

wherein:
each X is independently —CR³— or —N—;
Z is —O— or —NR⁵—;
X¹ is —CR¹— or —N—;
R^{1a} is —CN;
R^{1b} is hydrogen, F, Cl, Br, I, —CH₃, —CH₂CH₃, —CH₂F, —CHF₂, —CF₃, —OH, —OCF₃, —OCH₃, or —CN;
R¹ is hydrogen, F, Cl, Br, I, —CH₃, —CH₂CH₃, —CH₂F, —CHF₂, —CF₃, —CH₂CF₃, —OH, —OCF₃, —OCH₃, —OCH₂CH₃, or —OCH₂CF₃;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂;
each R⁴ is independently substituted or unsubstituted C₁-C₆alkyl, or C₁-C₆fluoroalkyl;
each R⁵ is independently hydrogen, substituted or unsubstituted C₁-C₆alkyl, C₁-C₆fluoroalkyl.

Embodiment 32. The compound of any one of embodiments 29-31, or a pharmaceutically acceptable salt or solvate thereof, wherein

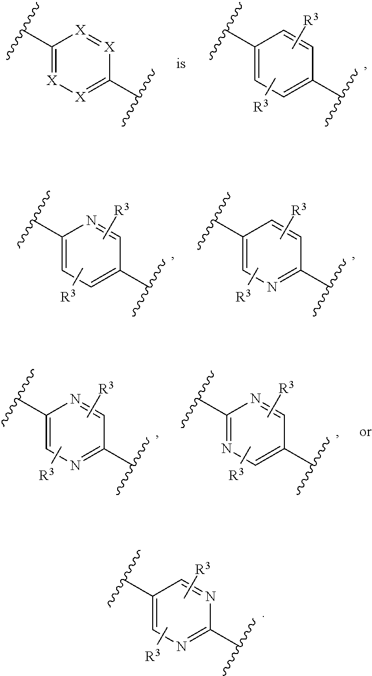

Embodiment 33. The compound of any one of embodiments 1-32, or a pharmaceutically acceptable salt or solvate thereof, wherein:
each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂, or —C(═O)NH(CH₃).

Embodiment 34. The compound of any one of embodiments 29-33, or a pharmaceutically acceptable salt or solvate thereof, wherein:

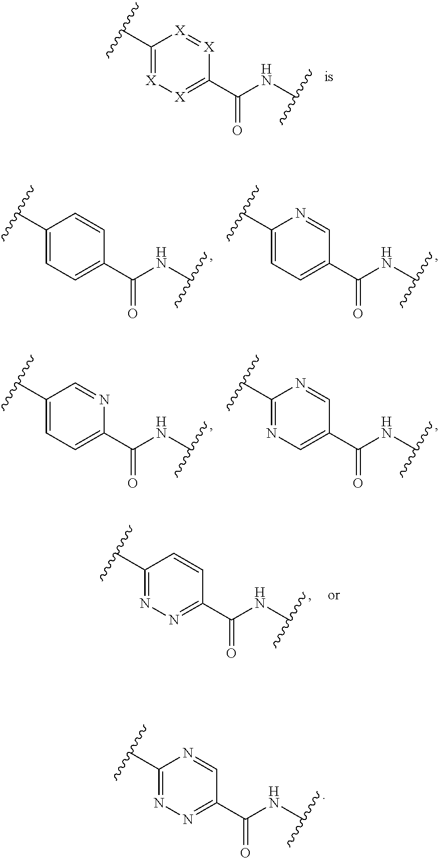

Embodiment 35. The compound of any one of embodiments 1-11 or 14, or a pharmaceutically acceptable salt or solvate thereof, wherein the head group and core of B-AR is:
N-(3-(3-fluoro-4-cyanophenoxy)-2,2,4,4-tetramethylcyclobutyl)acetamide-*;
N-(3-(3-chloro-4-cyanophenoxy)-2,2,4,4-tetramethylcyclobutyl)acetamide-*;
N-(3-(3-methoxy-4-cyanophenoxy)-2,2,4,4-tetramethylcyclobutyl)acetamide-*;
N-(3-(3-methyl-4-cyanophenoxy)-2,2,4,4-tetramethylcyclobutyl)acetamide-*;
N-(3-(3-trifluroromethyl-4-cyanophenoxy)-2,2,4,4-tetramethylcyclobutyl)acetamide-*;
N-(3-(3-trifluroromethoxy-4-cyanophenoxy)-2,2,4,4-tetramethylcyclobutyl)acetamide-*;
N-(3-((5-fluoro-6-cyanopyridin-3-yl)oxy)-2,2,4,4-tetramethylcyclobutyl)acetamide-*;
N-(3-((5-chloro-6-cyanopyridin-3-yl)oxy)-2,2,4,4-tetramethylcyclobutyl)acetamide-*;
N-(3-((5-methoxy-6-cyanopyridin-3-yl)oxy)-2,2,4,4-tetramethylcyclobutyl)acetamide-*;
N-(3-((5-methyl-6-cyanopyridin-3-yl)oxy)-2,2,4,4-tetramethylcyclobutyl)acetamide-*;
N-(3-((5-trifluroromethyl-6-cyanopyridin-3-yl)oxy)-2,2,4,4-tetramethylcyclobutyl)acetamide-*; or
N-(3-((5-trifluroromethoxy-6-cyanopyridin-3-yl)oxy)-2,2,4,4-tetramethylcyclobutyl)acetamide-*;
wherein acetamide-* is

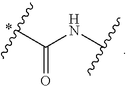 [SMILES: *NC(*)=O]

Embodiment 36. The compound of any one of embodiments 1-11 or 14, or a pharmaceutically acceptable salt or solvate thereof, wherein the head group and core of B-AR is:
N-(3-(3-chloro-4-cyanophenoxy)-2,2,4,4-tetramethylcyclobutyl)acetamide-*;
N-(3-(3-methoxy-4-cyanophenoxy)-2,2,4,4-tetramethylcyclobutyl)acetamide-*;
N-(3-(3-methyl-4-cyanophenoxy)-2,2,4,4-tetramethylcyclobutyl)acetamide-*; or
N-(3-(3-trifluroromethyl-4-cyanophenoxy)-2,2,4,4-tetramethylcyclobutyl)acetamide-*;
wherein acetamide-* is

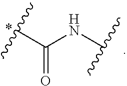 [SMILES: *NC(*)=O]

Embodiment 37. The compound of embodiment 35 or 36, or a pharmaceutically acceptable salt or solvate thereof, wherein B-AR further comprises a tail moiety that is covalently attached to position (*), wherein the tail moiety is a ring D that is phenyl, pyrimidinyl, pyrazinyl, pyridazinyl, triazinyl, triazolyl, pyrrolyl, imidazolyl, pyrazolyl, pyridyl, indolyl, isoindolyl, indolizinyl, benzimidazolyl, napthyl, quinolyl, isoquinolyl, quinoxalinyl, quinazolinyl, indazolyl, or benzotriazolyl; wherein ring D is optionally substituted with s R³.
s is 1, 2, or 3;
each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂, or —C(═O)NH(CH₃).

Embodiment 38. The compound of embodiment 35 or 36, or a pharmaceutically acceptable salt or solvate thereof, wherein ring D is phenyl, pyridyl, pyrimidinyl, pyrazinyl, pyridazinyl, or triazinyl; wherein ring D is optionally substituted with s R³; s is 1, 2, or 3;
each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, or —CN.

Embodiment 39. The compound of embodiment 1-11 or 14, or a pharmaceutically acceptable salt or solvate thereof, wherein B-AR has one of the following structures:

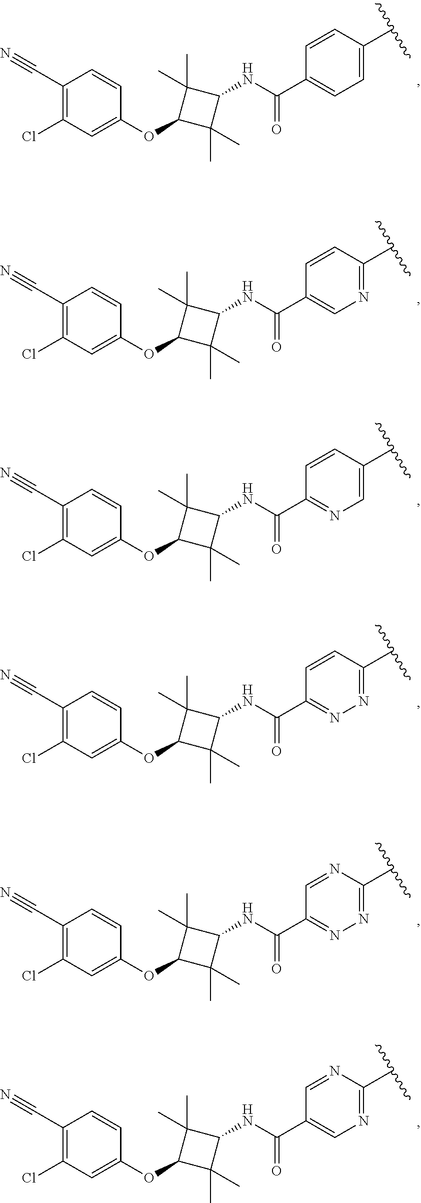 [SMILES: *c1ccc(C(=O)N[C@H]2C(C)(C)[C@H](Oc3ccc(C#N)c(Cl)c3)C2(C)C)cc1 ; *c1ccc(C(=O)N[C@H]2C(C)(C)[C@H](Oc3ccc(C#N)c(Cl)c3)C2(C)C)cn1 ; *c1ccc(C(=O)N[C@H]2C(C)(C)[C@H](Oc3ccc(C#N)c(Cl)c3)C2(C)C)nc1 ; *c1ccc(C(=O)N[C@H]2C(C)(C)[C@H](Oc3ccc(C#N)c(Cl)c3)C2(C)C)nn1 ; *c1ncc(C(=O)N[C@H]2C(C)(C)[C@H](Oc3ccc(C#N)c(Cl)c3)C2(C)C)nn1 ; *c1ncc(C(=O)N[C@H]2C(C)(C)[C@H](Oc3ccc(C#N)c(Cl)c3)C2(C)C)cn1]

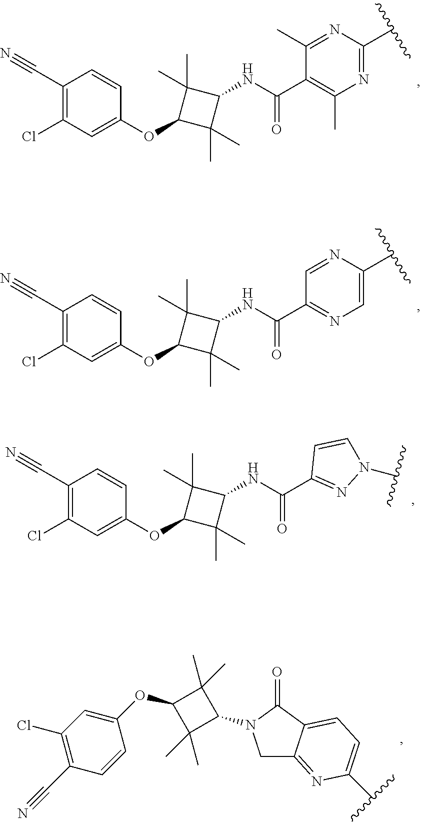 [SMILES: *c1nc(C)c(C(=O)N[C@H]2C(C)(C)[C@H](Oc3ccc(C#N)c(Cl)c3)C2(C)C)c(C)n1 ; *c1cnc(C(=O)N[C@H]2C(C)(C)[C@H](Oc3ccc(C#N)c(Cl)c3)C2(C)C)cn1 ; *n1ccc(C(=O)N[C@H]2C(C)(C)[C@H](Oc3ccc(C#N)c(Cl)c3)C2(C)C)n1 ; *c1ccc2c(n1)CN([C@H]1C(C)(C)[C@H](Oc3ccc(C#N)c(Cl)c3)C1(C)C)C2=O]

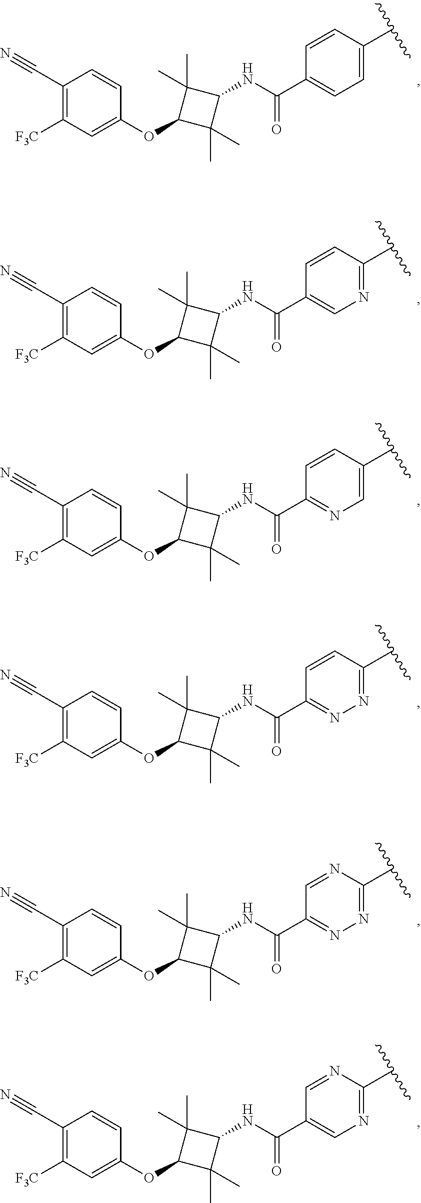 [SMILES: *c1ccc(C(=O)N[C@H]2C(C)(C)[C@H](Oc3ccc(C#N)c(C(F)(F)F)c3)C2(C)C)cc1 ; *c1ccc(C(=O)N[C@H]2C(C)(C)[C@H](Oc3ccc(C#N)c(C(F)(F)F)c3)C2(C)C)cn1 ; *c1ccc(C(=O)N[C@H]2C(C)(C)[C@H](Oc3ccc(C#N)c(C(F)(F)F)c3)C2(C)C)nc1 ; *c1ccc(C(=O)N[C@H]2C(C)(C)[C@H](Oc3ccc(C#N)c(C(F)(F)F)c3)C2(C)C)nn1 ; *c1ncc(C(=O)N[C@H]2C(C)(C)[C@H](Oc3ccc(C#N)c(C(F)(F)F)c3)C2(C)C)nn1 ; *c1ncc(C(=O)N[C@H]2C(C)(C)[C@H](Oc3ccc(C#N)c(C(F)(F)F)c3)C2(C)C)cn1]

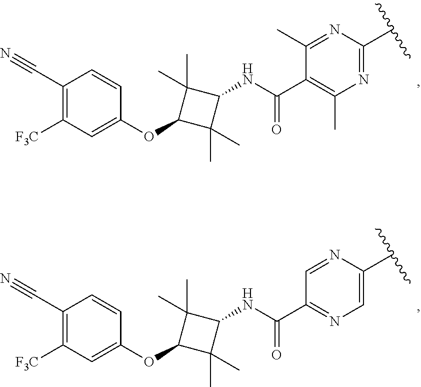 [SMILES: *c1nc(C)c(C(=O)N[C@H]2C(C)(C)[C@H](Oc3ccc(C#N)c(C(F)(F)F)c3)C2(C)C)c(C)n1 ; *c1cnc(C(=O)N[C@H]2C(C)(C)[C@H](Oc3ccc(C#N)c(C(F)(F)F)c3)C2(C)C)cn1]

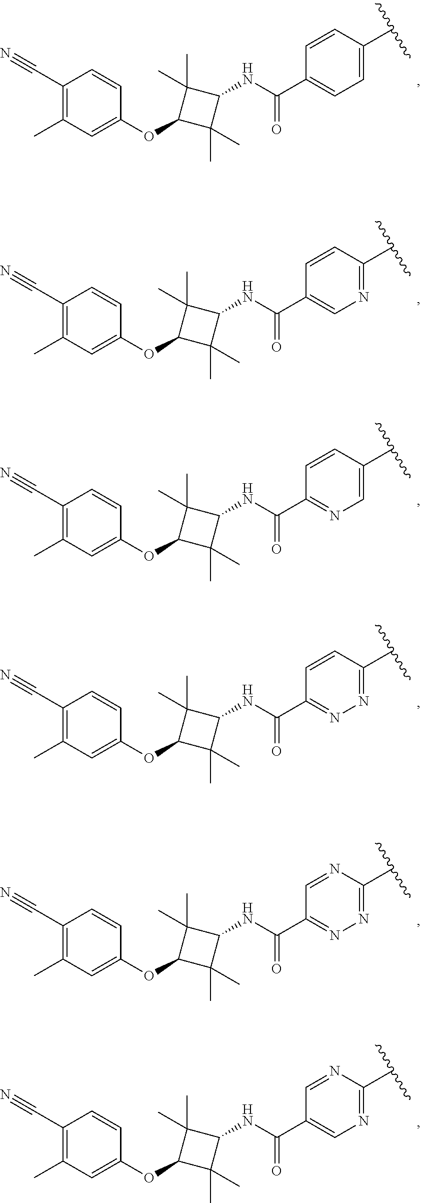 [SMILES: *c1ccc(C(=O)N[C@H]2C(C)(C)[C@H](Oc3ccc(C#N)c(C)c3)C2(C)C)cc1 ; *c1ccc(C(=O)N[C@H]2C(C)(C)[C@H](Oc3ccc(C#N)c(C)c3)C2(C)C)cn1 ; *c1ccc(C(=O)N[C@H]2C(C)(C)[C@H](Oc3ccc(C#N)c(C)c3)C2(C)C)nc1 ; *c1ccc(C(=O)N[C@H]2C(C)(C)[C@H](Oc3ccc(C#N)c(C)c3)C2(C)C)nn1 ; *c1ncc(C(=O)N[C@H]2C(C)(C)[C@H](Oc3ccc(C#N)c(C)c3)C2(C)C)nn1 ; *c1ncc(C(=O)N[C@H]2C(C)(C)[C@H](Oc3ccc(C#N)c(C)c3)C2(C)C)cn1]

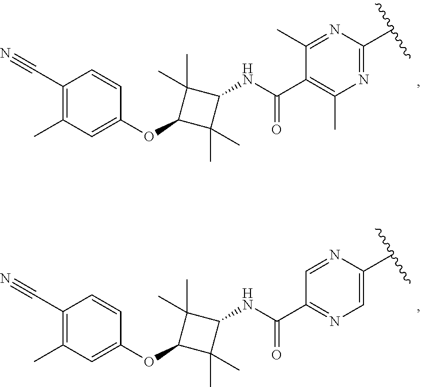 [SMILES: *c1nc(C)c(C(=O)N[C@H]2C(C)(C)[C@H](Oc3ccc(C#N)c(C)c3)C2(C)C)c(C)n1 ; *c1cnc(C(=O)N[C@H]2C(C)(C)[C@H](Oc3ccc(C#N)c(C)c3)C2(C)C)cn1]

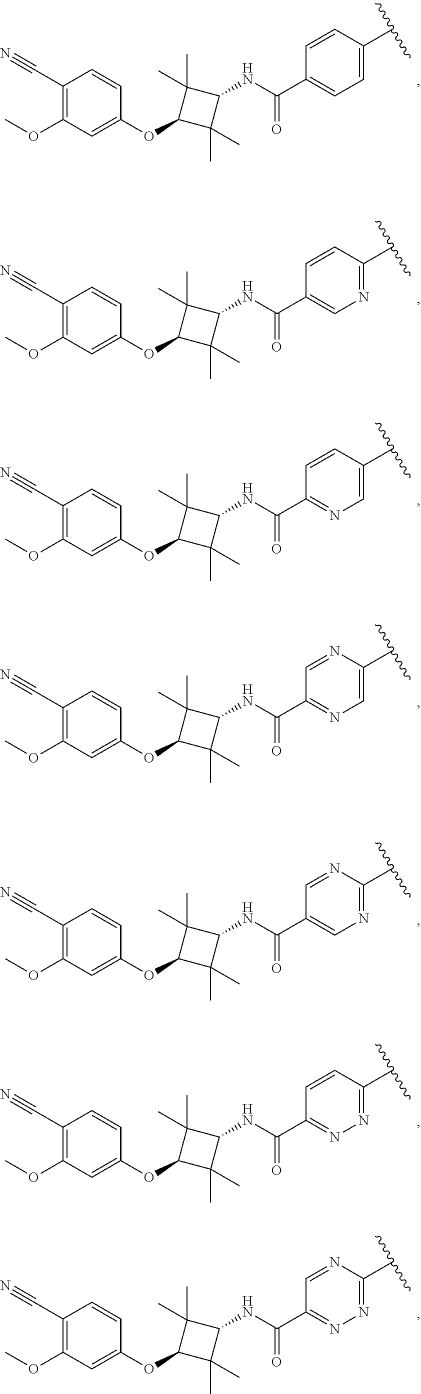

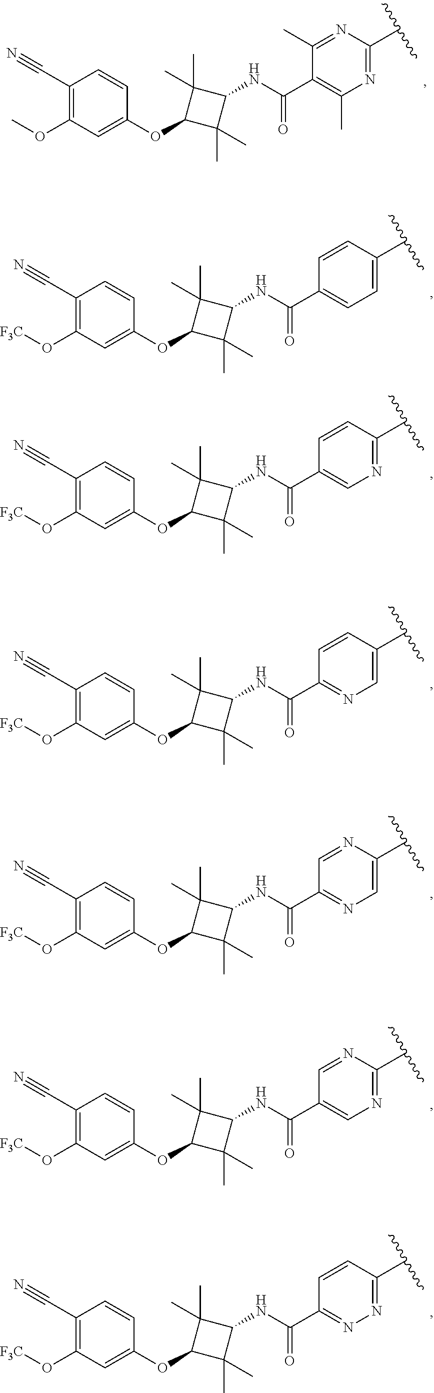

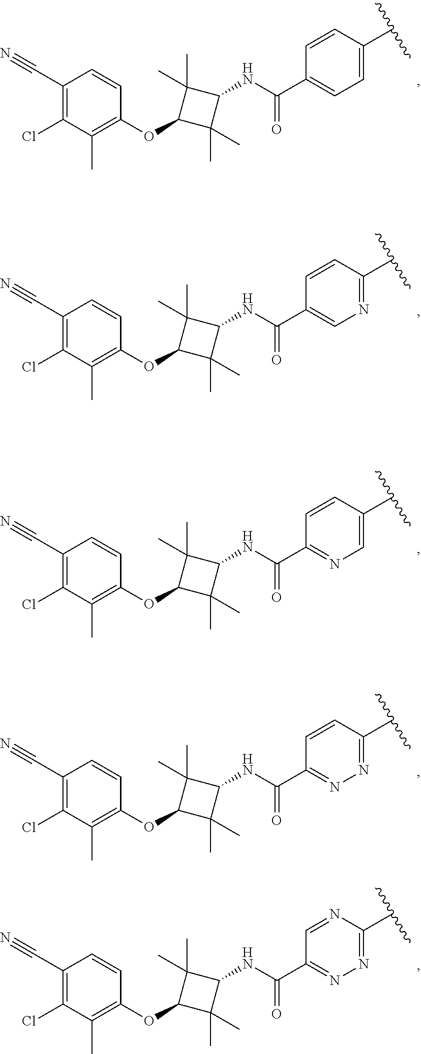 [SMILES: *c1ccc(C(=O)N[C@H]2C(C)(C)[C@H](Oc3ccc(C#N)c(Cl)c3C)C2(C)C)cc1 ; *c1ccc(C(=O)N[C@H]2C(C)(C)[C@H](Oc3ccc(C#N)c(Cl)c3C)C2(C)C)cn1 ; *c1ccc(C(=O)N[C@H]2C(C)(C)[C@H](Oc3ccc(C#N)c(Cl)c3C)C2(C)C)nc1 ; *c1ccc(C(=O)N[C@H]2C(C)(C)[C@H](Oc3ccc(C#N)c(Cl)c3C)C2(C)C)nn1 ; *c1ncc(C(=O)N[C@H]2C(C)(C)[C@H](Oc3ccc(C#N)c(Cl)c3C)C2(C)C)nn1]

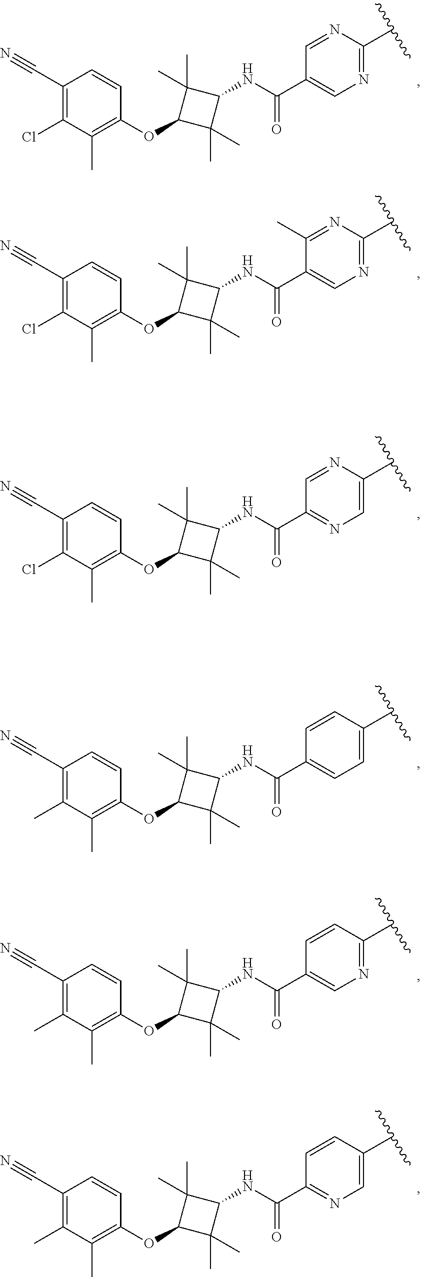 [SMILES: *c1ncc(C(=O)N[C@H]2C(C)(C)[C@H](Oc3ccc(C#N)c(Cl)c3C)C2(C)C)cn1 ; *c1ncc(C(=O)N[C@H]2C(C)(C)[C@H](Oc3ccc(C#N)c(Cl)c3C)C2(C)C)c(C)n1 ; *c1cnc(C(=O)N[C@H]2C(C)(C)[C@H](Oc3ccc(C#N)c(Cl)c3C)C2(C)C)cn1 ; *c1ccc(C(=O)N[C@H]2C(C)(C)[C@H](Oc3ccc(C#N)c(C)c3C)C2(C)C)cc1 ; *c1ccc(C(=O)N[C@H]2C(C)(C)[C@H](Oc3ccc(C#N)c(C)c3C)C2(C)C)cn1 ; *c1ccc(C(=O)N[C@H]2C(C)(C)[C@H](Oc3ccc(C#N)c(C)c3C)C2(C)C)nc1]

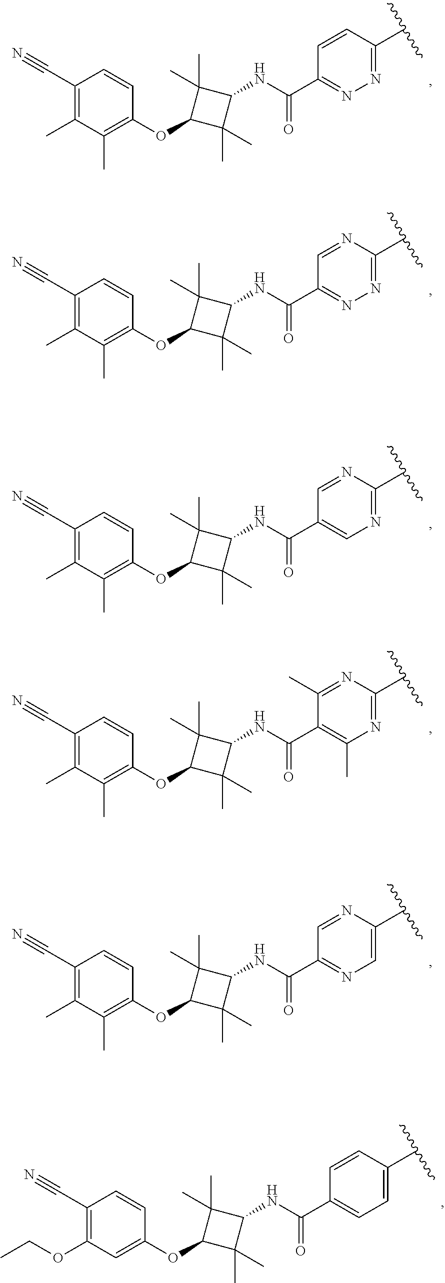 [SMILES: *c1ccc(C(=O)N[C@H]2C(C)(C)[C@H](Oc3ccc(C#N)c(C)c3C)C2(C)C)nn1 ; *c1ncc(C(=O)N[C@H]2C(C)(C)[C@H](Oc3ccc(C#N)c(C)c3C)C2(C)C)nn1 ; *c1ncc(C(=O)N[C@H]2C(C)(C)[C@H](Oc3ccc(C#N)c(C)c3C)C2(C)C)cn1 ; *c1nc(C)c(C(=O)N[C@H]2C(C)(C)[C@H](Oc3ccc(C#N)c(C)c3C)C2(C)C)c(C)n1 ; *c1cnc(C(=O)N[C@H]2C(C)(C)[C@H](Oc3ccc(C#N)c(C)c3C)C2(C)C)cn1 ; *c1ccc(C(=O)N[C@H]2C(C)(C)[C@H](Oc3ccc(C#N)c(OCC)c3)C2(C)C)cc1]

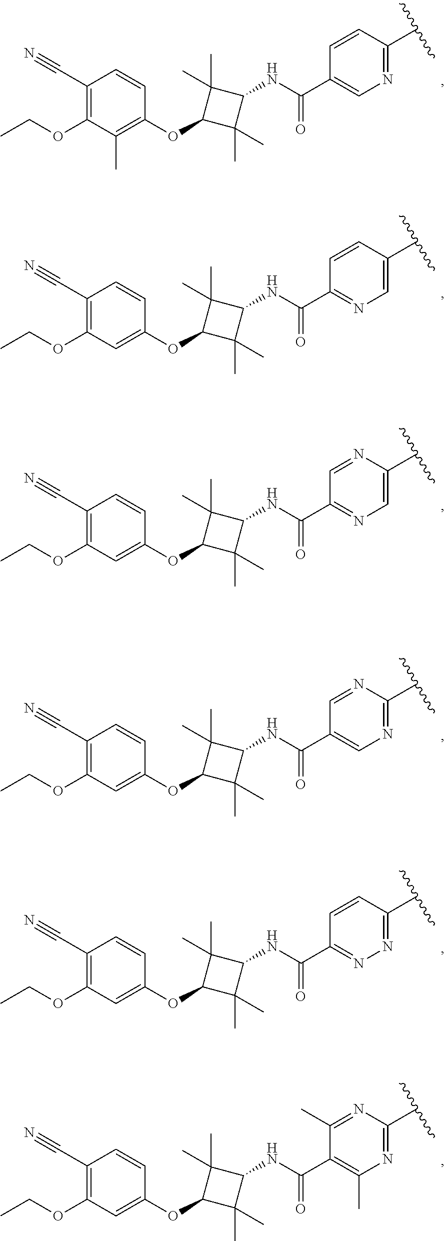 [SMILES: *c1ccc(C(=O)N[C@H]2C(C)(C)[C@H](Oc3ccc(C#N)c(OCC)c3C)C2(C)C)cn1 ; *c1ccc(C(=O)N[C@H]2C(C)(C)[C@H](Oc3ccc(C#N)c(OCC)c3)C2(C)C)nc1 ; *c1cnc(C(=O)N[C@H]2C(C)(C)[C@H](Oc3ccc(C#N)c(OCC)c3)C2(C)C)cn1 ; *c1ncc(C(=O)N[C@H]2C(C)(C)[C@H](Oc3ccc(C#N)c(OCC)c3)C2(C)C)cn1 ; *c1ccc(C(=O)N[C@H]2C(C)(C)[C@H](Oc3ccc(C#N)c(OCC)c3)C2(C)C)nn1 ; *c1nc(C)c(C(=O)N[C@H]2C(C)(C)[C@H](Oc3ccc(C#N)c(OCC)c3)C2(C)C)c(C)n1]

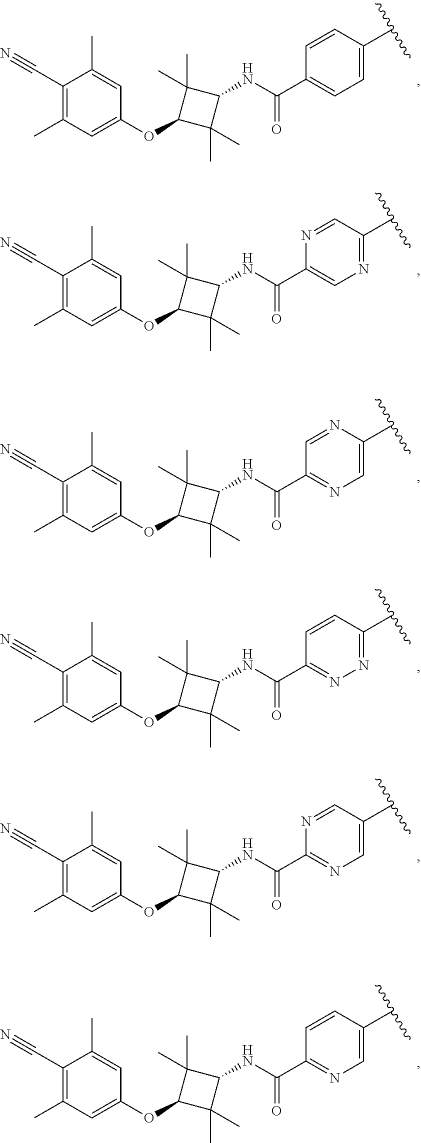 [SMILES: *c1ccc(C(=O)N[C@H]2C(C)(C)[C@H](Oc3cc(C)c(C#N)c(C)c3)C2(C)C)cc1 ; *c1cnc(C(=O)N[C@H]2C(C)(C)[C@H](Oc3cc(C)c(C#N)c(C)c3)C2(C)C)cn1 ; *c1cnc(C(=O)N[C@H]2C(C)(C)[C@H](Oc3cc(C)c(C#N)c(C)c3)C2(C)C)cn1 ; *c1ccc(C(=O)N[C@H]2C(C)(C)[C@H](Oc3cc(C)c(C#N)c(C)c3)C2(C)C)nn1 ; *c1cnc(C(=O)N[C@H]2C(C)(C)[C@H](Oc3cc(C)c(C#N)c(C)c3)C2(C)C)nc1 ; *c1ccc(C(=O)N[C@H]2C(C)(C)[C@H](Oc3cc(C)c(C#N)c(C)c3)C2(C)C)nc1]

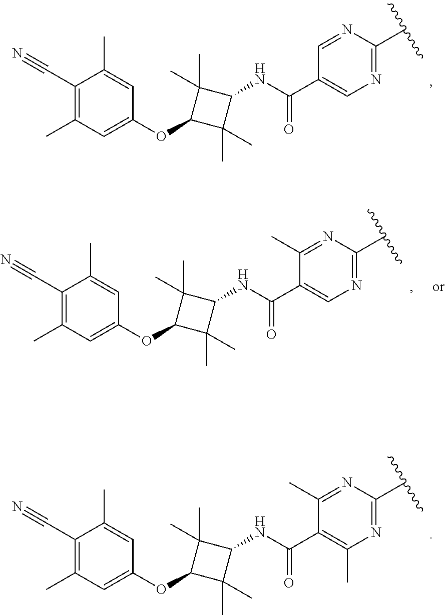 [SMILES: *c1ncc(C(=O)N[C@H]2C(C)(C)[C@H](Oc3cc(C)c(C#N)c(C)c3)C2(C)C)cn1 ; *c1ncc(C(=O)N[C@H]2C(C)(C)[C@H](Oc3cc(C)c(C#N)c(C)c3)C2(C)C)c(C)n1 ; *c1nc(C)c(C(=O)N[C@H]2C(C)(C)[C@H](Oc3cc(C)c(C#N)c(C)c3)C2(C)C)c(C)n1]

Embodiment 39a. The compound of embodiment 39, or a pharmaceutically acceptable salt or solvate thereof, wherein B-AR has one of the following structures of embodiment 39, wherein

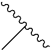

is halide.

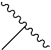

is halide.

Embodiment 39b. The compound of embodiment 39a, or a pharmaceutically acceptable salt or solvate thereof, wherein B-AR has one of the following structures of embodiment 39a, wherein

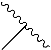

### is Cl, Br or I.

Embodiment 39c. The compound of embodiment 39a, or a pharmaceutically acceptable salt or solvate thereof, wherein B-AR has one of the following structures of embodiment 39a, wherein

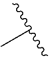

### is Cl.

Embodiment 39d. The compound of embodiment 39a, or a pharmaceutically acceptable salt or solvate thereof, wherein B-AR has one of the following structures of embodiment 39a, wherein

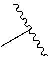

### is H.

Embodiment 39e. A compound, or a pharmaceutically acceptable salt or solvate thereof, that has one of the following structures:

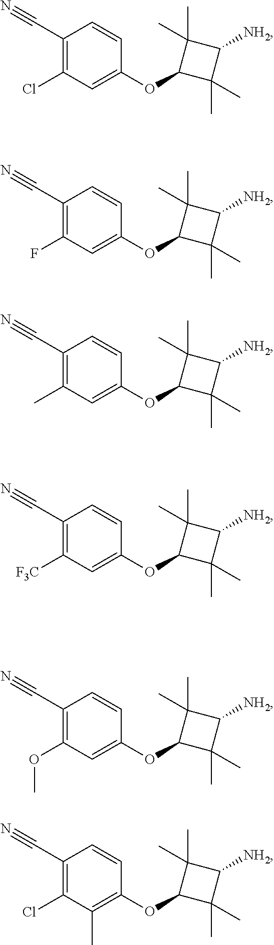

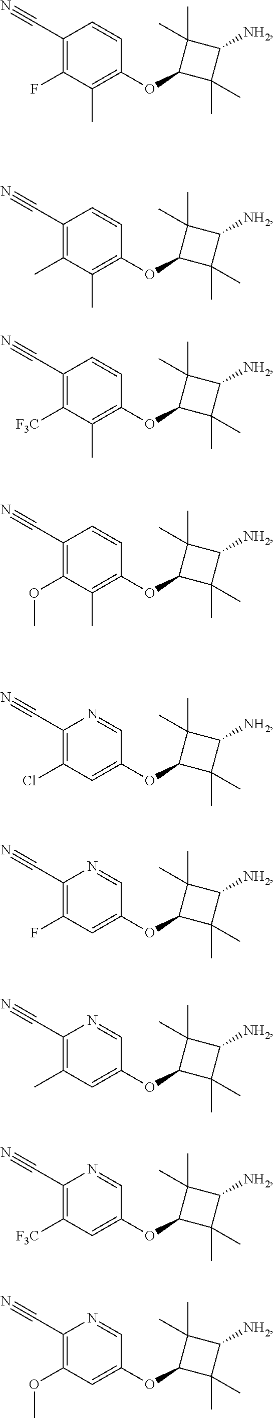

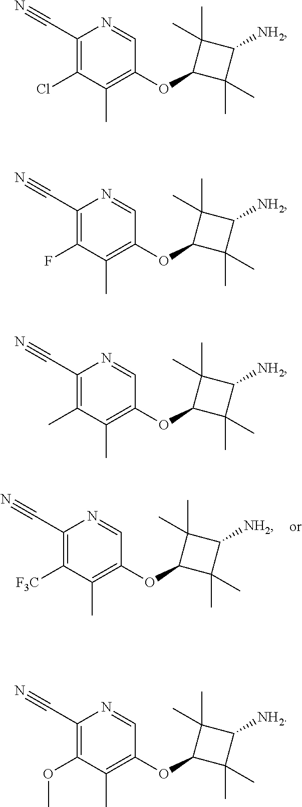 [SMILES: Cc1c(O[C@H]2C(C)(C)[C@H](N)C2(C)C)cnc(C#N)c1Cl ; Cc1c(O[C@H]2C(C)(C)[C@H](N)C2(C)C)cnc(C#N)c1F ; Cc1c(O[C@H]2C(C)(C)[C@H](N)C2(C)C)cnc(C#N)c1C ; Cc1c(O[C@H]2C(C)(C)[C@H](N)C2(C)C)cnc(C#N)c1C(F)(F)F ; COc1c(C#N)ncc(O[C@H]2C(C)(C)[C@H](N)C2(C)C)c1C]

Embodiment 39f. A compound, or a pharmaceutically acceptable salt or solvate thereof, that has one of the following structures:

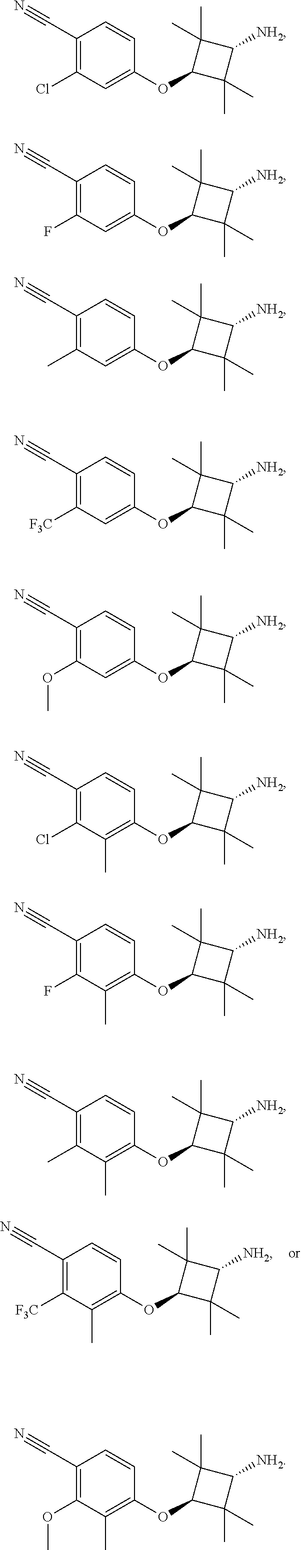

Embodiment 39g. A compound, or a pharmaceutically acceptable salt or solvate thereof, that has one of the following structures:

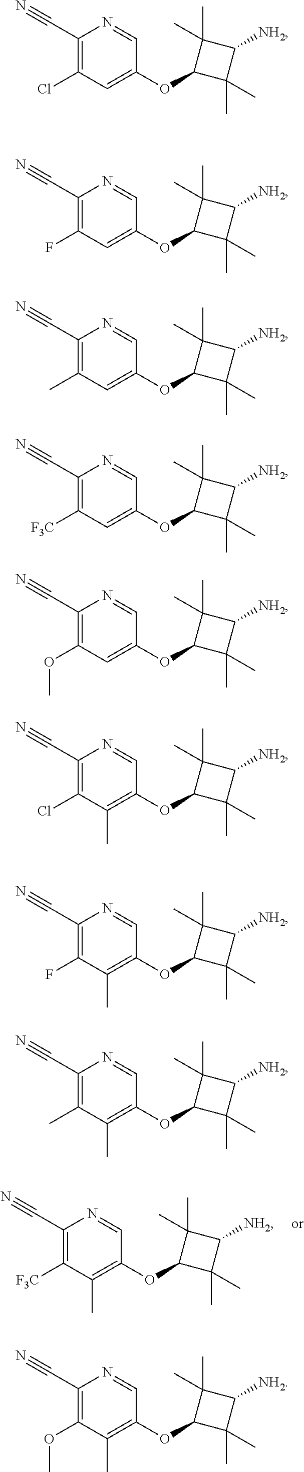

Embodiment 40. The compound of any one of embodiments 14-39, or a pharmaceutically acceptable salt or solvate thereof, wherein SB-CBP/p300 binds in the acetyl-lysine (KAc) binding site of the bromodomain of CBP/p300.

Embodiment 41. The compound of any one of embodiments 14-39, or a pharmaceutically acceptable salt or solvate thereof, wherein SB-CBP/p300 comprises an acetyl-lysine mimetic moiety that binds in the acetyl-lysine (KAc) binding site of the bromodomain of CBP/p300.

Embodiment 42. The compound of embodiment 41, or a pharmaceutically acceptable salt or solvate thereof, wherein L is covalently attached at a position of a) that is solvent exposed when a) binds the KAc binding site of the bromodomain of CBP/p300.

Embodiment 43. The compound of embodiment 41, or a pharmaceutically acceptable salt or solvate thereof, wherein the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300; and the head group of B-AR forms hydrogen bonds with the side chains of Gln 711 and Arg 752 of the LBD of AR.

Embodiment 44. The compound of any one of embodiments 14-43, or a pharmaceutically acceptable salt or solvate thereof, wherein SB-CBP/p300 further comprises a moiety that interacts with Arg1173 in the bromodomain of CBP or Asn1137 in the bromodomain of p300.

Embodiment 45. The compound of any one of embodiments 14-44, or a pharmaceutically acceptable salt or solvate thereof, wherein SB-CBP/p300 further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2).

Embodiment 46. The compound of embodiment 41-45, or a pharmaceutically acceptable salt or solvate thereof, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

Embodiment 47. The compound of embodiment 45 or 46, or a pharmaceutically acceptable salt or solvate thereof, wherein L is covalently attached to SB-CBP/p300 on:
the acetyl-lysine mimetic moiety; or
the moiety that occupies the lipophilic shelf (LPF) region of the bromodomain of CBP/p300, if present; or
the moiety that occupies the BC Loop region of the bromodomain of CBP/p300, if present;
wherein L is covalently attached to SB-p300/CBP at a position that does not interfere with the binding of the acetyl-lysine mimetic moiety in the acetylated lysine (KAc) binding site of CBP/p300.

Embodiment 48. The compound of any one of embodiments 1-47, or a pharmaceutically acceptable salt or solvate thereof, wherein the moiety comprising an acetyl-lysine mimetic comprises a moiety selected from:

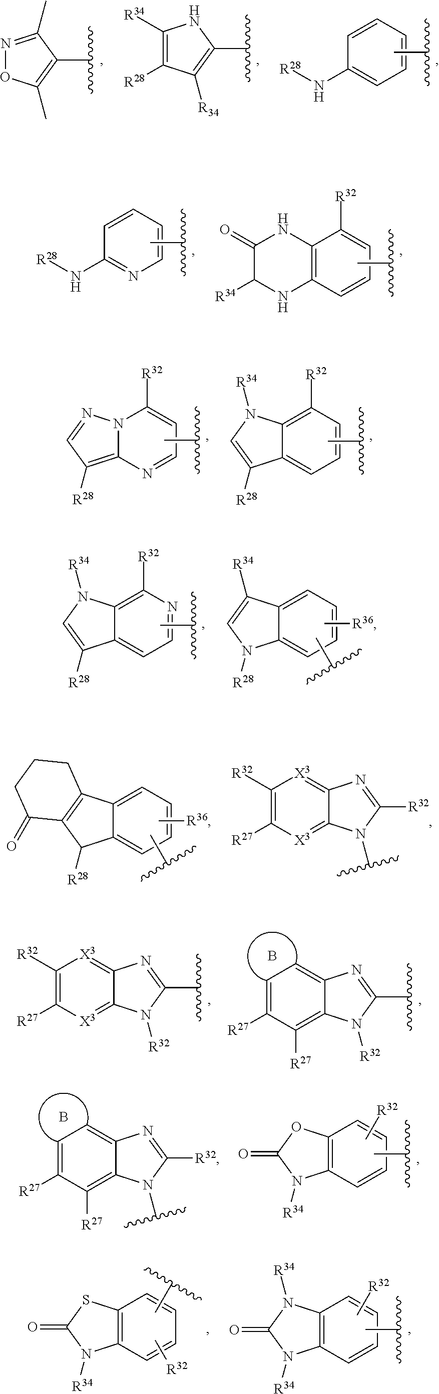

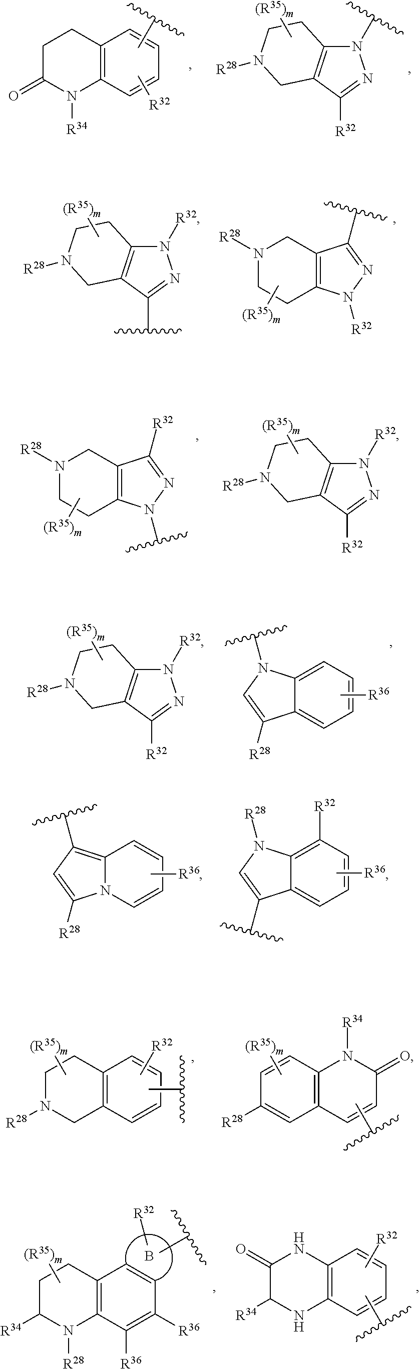

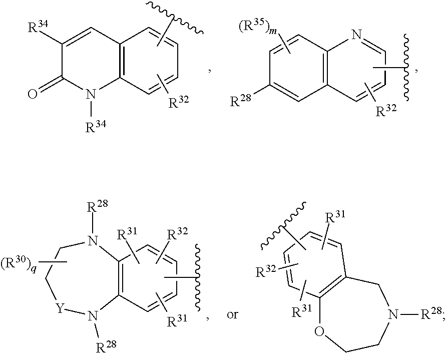

each R³² is independently an optional moiety that occupies the lipophilic shelf (LPF) region of the bromodomain of CBP/p300;
or each R³² is independently an optional moiety that occupies the BC Loop region of the bromodomain of CBP/p300;

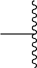

is the point of attachment to L that covalently connects SB-CBP/p300 to B-AR;
or R³² comprises

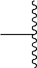

and L that covalently connects SB-CBP/p300 to B-AR is attached to R³²;
each R²⁸ is independently hydrogen, substituted or unsubstituted C₁-C₆alkyl, substituted or unsubstituted C₁-C₆fluoroalkyl, —C(═O)R^{b}, or —C(═O)N(R^{b})₂;
each R³⁴ is independently hydrogen or substituted or unsubstituted C₁-C₆alkyl;
each R³⁵ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl, —CN, —OH, —OR^{a}, or —N(R^{b})₂;
m is 0, 1, 2, 3, or 4;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each X³ is independently CR²⁷ or N;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
each R³⁶ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
Ring B is a fused substituted or unsubstituted 5 or 6 membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 49. The compound of embodiment 45-48, or a pharmaceutically acceptable salt or solvate thereof, wherein the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl;
or R³² is

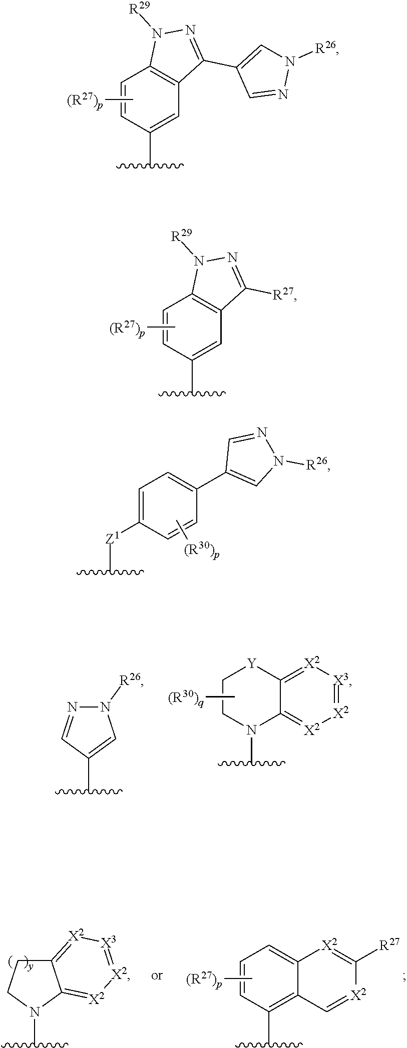

y is 1 or 2;
Z¹ is —NR^{c}—, —O—, or —S—;
R^{c} is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each X² is independently —CR³⁰— or —N—;
each X³ is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Y is —C(R³⁰)₂— or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
or R³² is -L-C;
L is substituted or unsubstituted C₁-C₆alkyl or substituted or unsubstituted C₁-C₆heteroalkyl;
C is substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 50. The compound of any one of embodiments 45-49, or a pharmaceutically acceptable salt or solvate thereof, wherein the moiety that occupies the BC Loop region of the bromodomain of CBP/p300 is R³², wherein:
R³² is

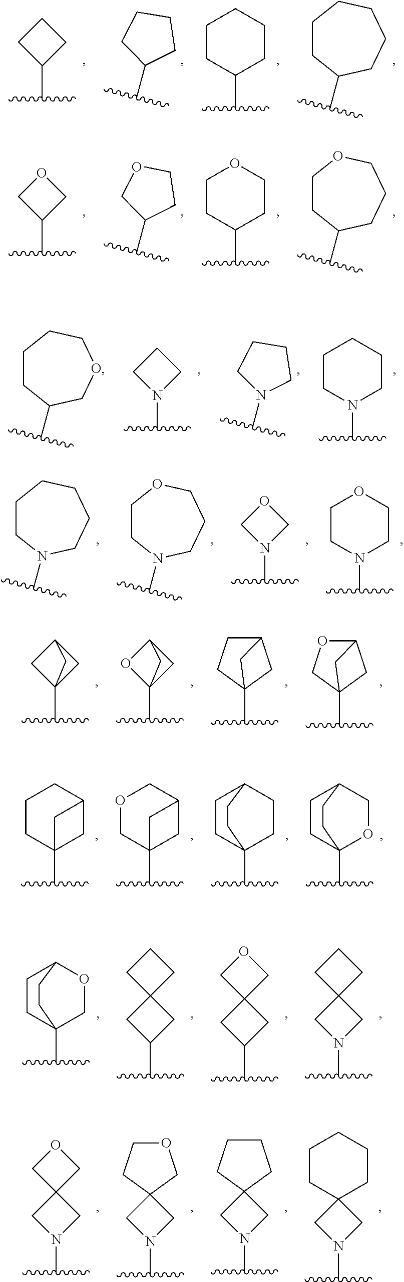

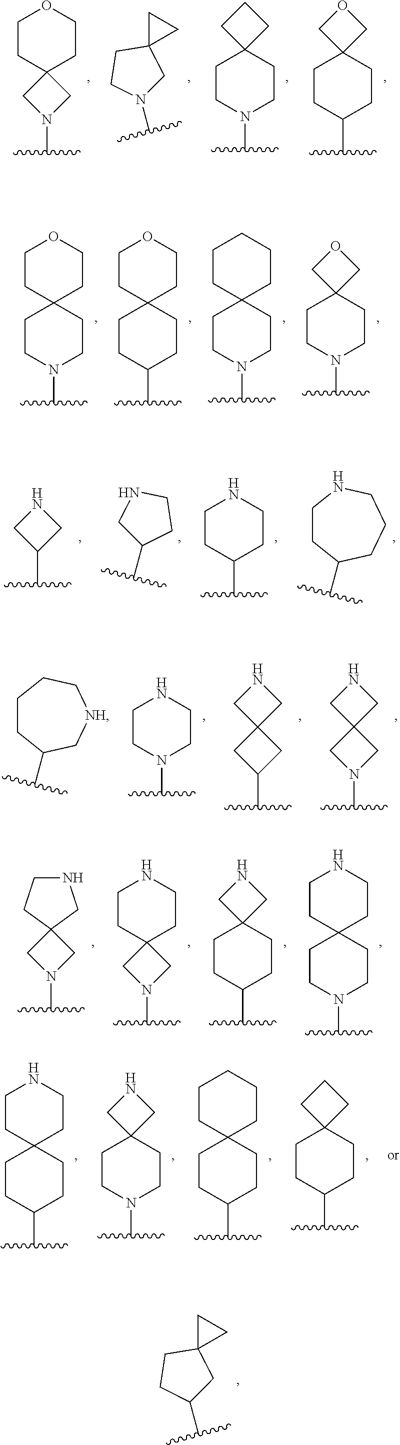

each of which is substituted or unsubstituted.

Embodiment 51. The compound of any one of embodiments 41-50, or a pharmaceutically acceptable salt or solvate thereof, wherein the moiety comprising an acetyl-lysine mimetic comprises

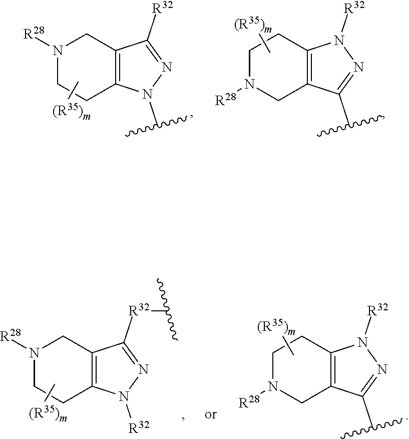

Embodiment 52. The compound of embodiment 51, or a pharmaceutically acceptable salt or solvate thereof, wherein:

R³² is

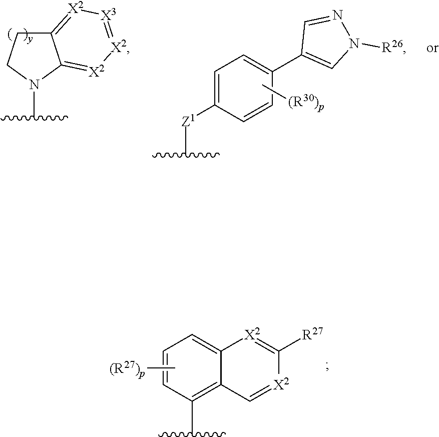

R²⁷ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and
each R²⁷ is independently hydrogen, substituted or unsubstituted C₁-C₆alkyl, substituted or unsubstituted C₁-C₆fluoroalkyl, substituted or unsubstituted C₁-C₆heteroalkyl, —CN, —OH, or —OR^{a}; and
each R^{a} is independently substituted or unsubstituted C₁-C₆alkyl.

Embodiment 53. The compound of embodiment 52, or a pharmaceutically acceptable salt or solvate thereof, wherein:
each R²⁷ is independently hydrogen, —CH₃, —CH₂CH₃, —F, —CHF₂, —CF₃, —CN, —OH, —OCH₃, cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

Embodiment 54. The compound of any one of embodiments 51-53, or a pharmaceutically acceptable salt or solvate thereof, wherein the moiety comprising an acetyl-lysine mimetic comprises

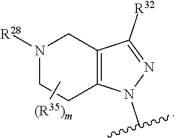

Embodiment 55. The compound of any one of embodiments 51-53, or a pharmaceutically acceptable salt or solvate thereof, wherein the moiety comprising an acetyl-lysine mimetic comprises

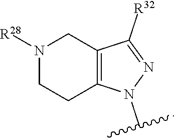

Embodiment 56. The compound of any one of embodiments 51-55, or a pharmaceutically acceptable salt or solvate thereof, wherein:

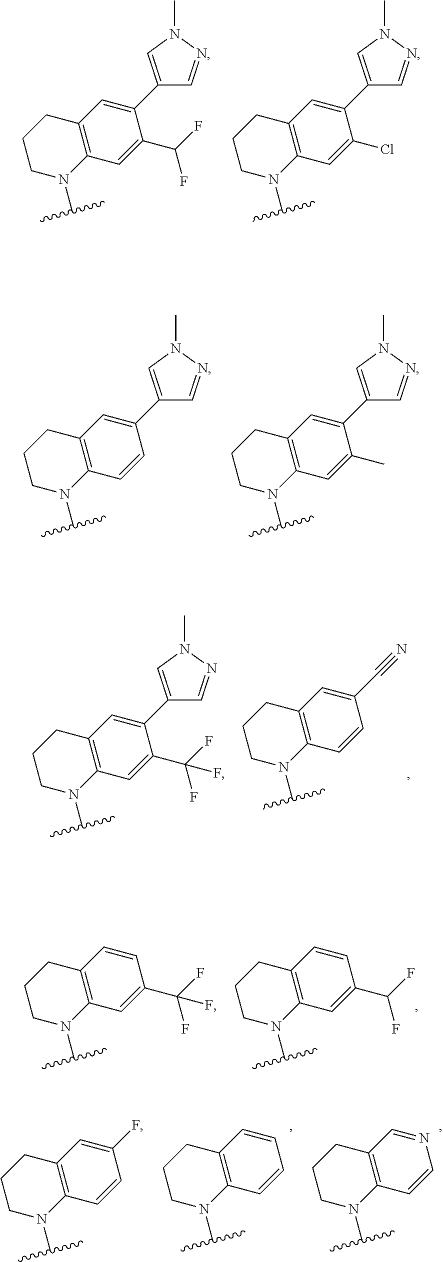

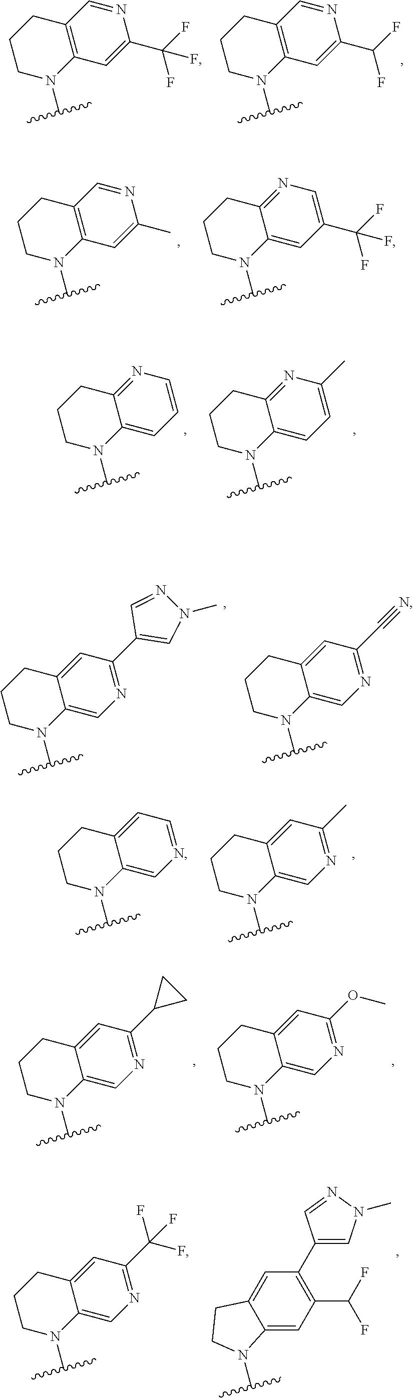

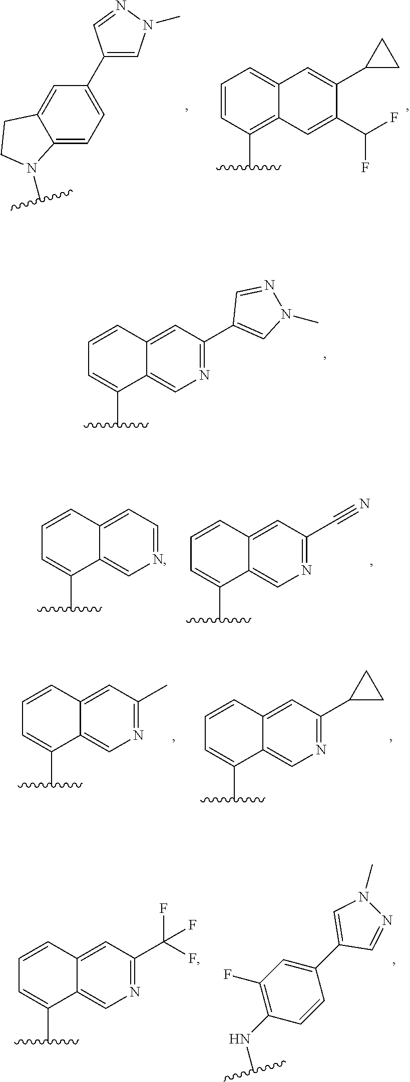

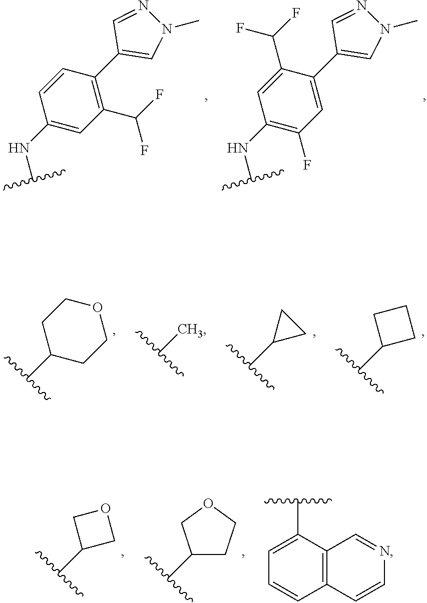

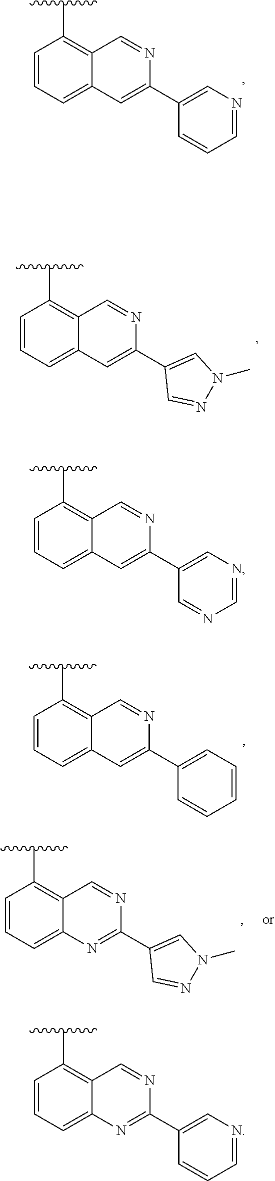 [SMILES: *c1cccc2cc(-c3cccnc3)ncc12 ; *c1cccc2cc(-c3cnn(C)c3)ncc12 ; *c1cccc2cc(-c3cncnc3)ncc12 ; *c1cccc2cc(-c3ccccc3)ncc12 ; *c1cccc2nc(-c3cnn(C)c3)ncc12 ; *c1cccc2nc(-c3cccnc3)ncc12]

Embodiment 57. The compound of any one of embodiments 41-50, or a pharmaceutically acceptable salt or solvate thereof, wherein the moiety comprising an acetyl-lysine mimetic comprises

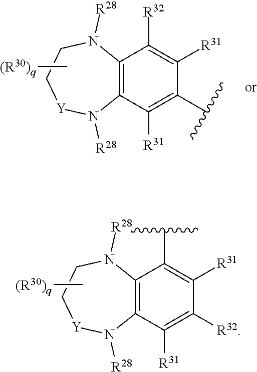

Embodiment 58. The compound of embodiment 57, or a pharmaceutically acceptable salt or solvate thereof, wherein the moiety comprising an acetyl-lysine mimetic comprises

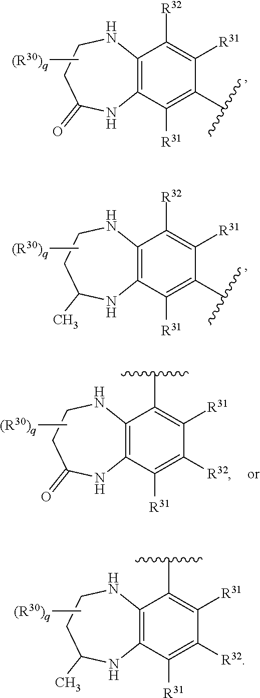

Embodiment 59. The compound of embodiment 57, or a pharmaceutically acceptable salt or solvate thereof, wherein the moiety comprising an acetyl-lysine mimetic comprises

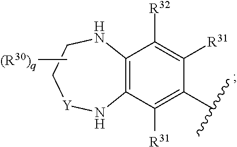

wherein:

R³² is

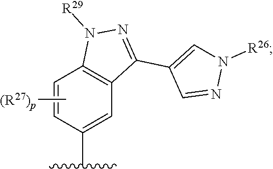

and
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂.

Embodiment 60. The compound of any one of embodiments 57-59, or a pharmaceutically acceptable salt or solvate thereof, wherein:
R³² is

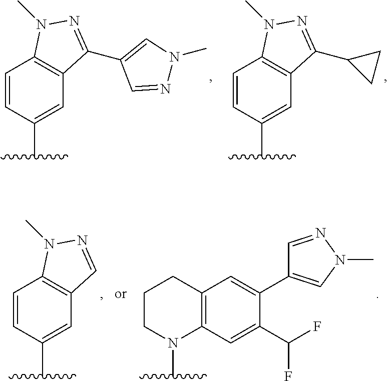

Embodiment 61. The compound of any one of embodiments 41-50, or a pharmaceutically acceptable salt or solvate thereof, wherein the moiety comprising an acetyl-lysine mimetic comprises

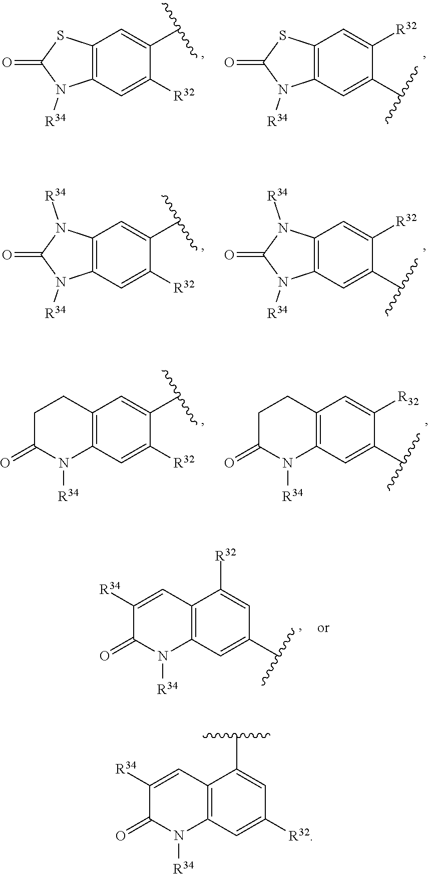

Embodiment 62. The compound of embodiment 61, or a pharmaceutically acceptable salt or solvate thereof, wherein the moiety comprising an acetyl-lysine mimetic comprises

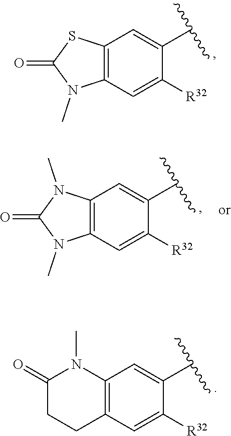

Embodiment 63. The compound of any one of embodiments 61-62, or a pharmaceutically acceptable salt or solvate thereof, wherein:
R³² is

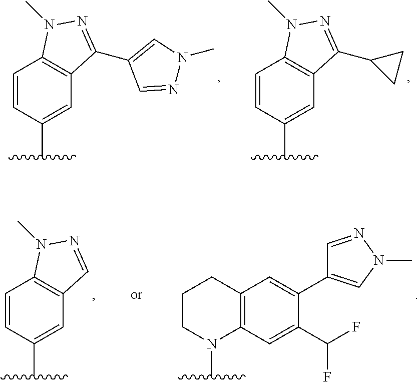 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

Embodiment 64. The compound of embodiment 61, or a pharmaceutically acceptable salt or solvate thereof, wherein the moiety comprising an acetyl-lysine mimetic comprises:

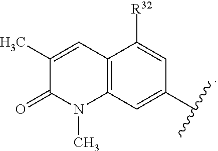

Embodiment 65. The compound of embodiment 61 or 64, or a pharmaceutically acceptable salt or solvate thereof, wherein:
R³² is

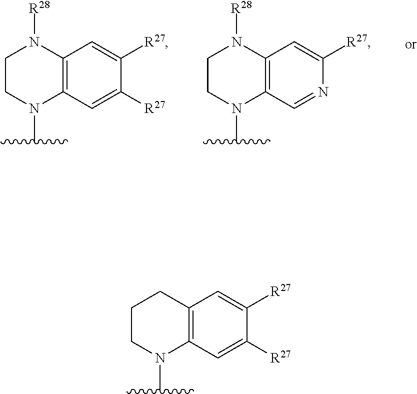

Embodiment 66. The compound of any one of embodiments 61, 62, 64, or 65, or a pharmaceutically acceptable salt or solvate thereof, wherein:
R³² is

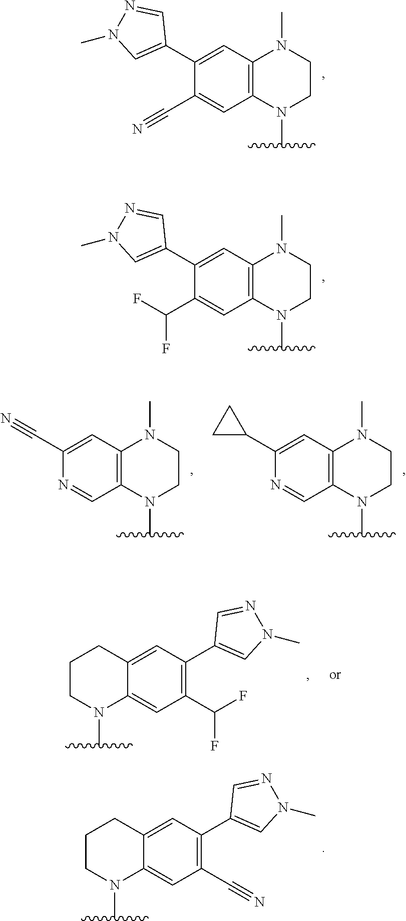

Embodiment 67. The compound of any one of embodiments 41-50, or a pharmaceutically acceptable salt or solvate thereof, wherein the moiety comprising an acetyl-lysine mimetic comprises

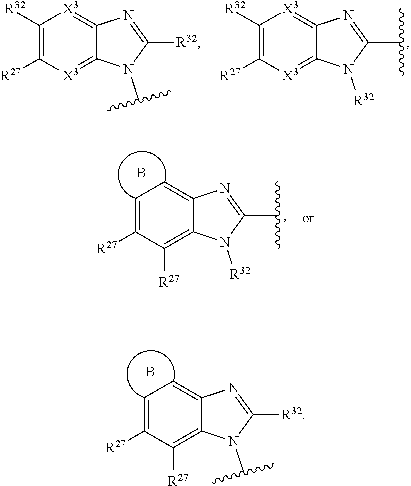

Embodiment 68. The compound of embodiment 67, or a pharmaceutically acceptable salt or solvate thereof, wherein the moiety comprising an acetyl-lysine mimetic comprises

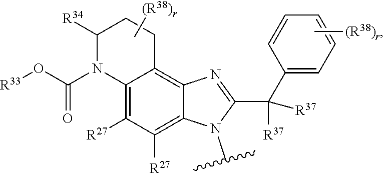

wherein:
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
R³³ is hydrogen, substituted or unsubstituted C₁-C₆alkyl, substituted or unsubstituted C₁-C₆fluoroalkyl, or substituted or unsubstituted C₁-C₆heteroalkyl;
R³⁴ is hydrogen or substituted or unsubstituted C₁-C₆alkyl;
each R³⁸ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, —CN, —NO₂, —OH, —OR^{a}, —OC(═O)R^{a}, —OC(═O)N(R^{b})₂, —OC(═O)OR^{a}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
r is 0, 1, 2, 3, or 4;

Embodiment 69. The compound of embodiment 67, or a pharmaceutically acceptable salt or solvate thereof, wherein:

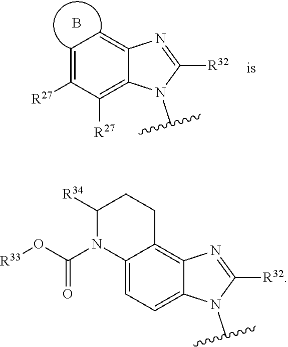

Embodiment 70. The compound of embodiment 67, or a pharmaceutically acceptable salt or solvate thereof, wherein the moiety comprising an acetyl-lysine mimetic comprises

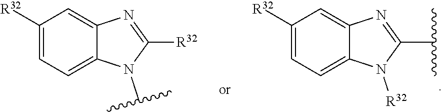

Embodiment 71. The compound of any one of embodiments 67-70, or a pharmaceutically acceptable salt or solvate thereof, wherein:
R³² is

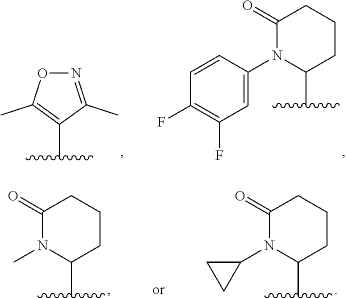

Embodiment 72. The compound of any one of embodiments 67-70, or a pharmaceutically acceptable salt or solvate thereof, wherein:

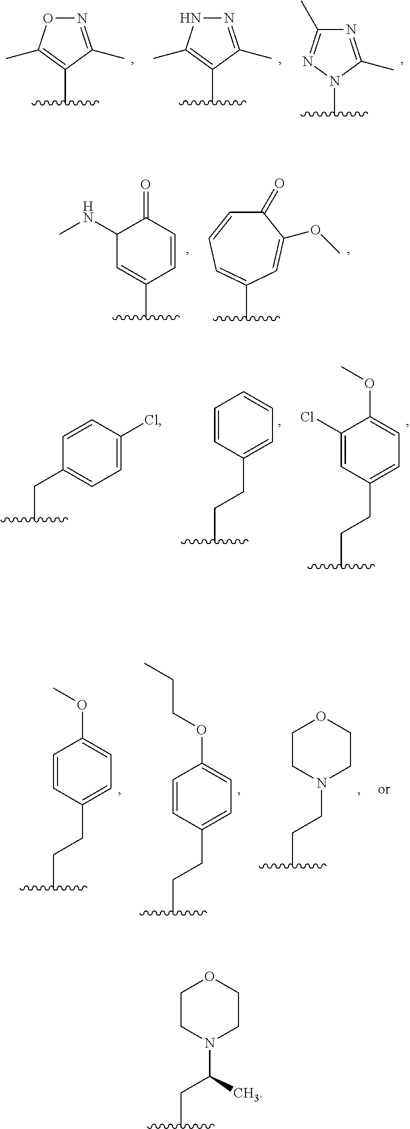

Embodiment 73. A heterobifunctional conditional inhibitor compound of Formula (I) or a pharmaceutically acceptable salt or solvate thereof:

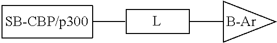

wherein:
SB-CBP/p300 is a silent binder of the bromodomain of human CREB-binding protein (CBP) or human E1A-binding protein p300 (p300) (CBP/p300), wherein the binder of CBP/p300 comprises:
   an acetyl-lysine mimetic moiety that binds in the acetyl-lysine (KAc) binding site of the bromodomain of human CREB-binding protein (CBP) or human E1A-binding protein p300 (p300) (CBP/p300) and has the structure:

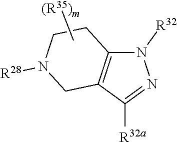

R²⁸ is —C(═O)CH₃, —C(═O)CH₂CH₃, —C(═O)NH₂, —C(═O)NH(CH₃); or —C(═O)NH(CH₂CH₃);
R³² is a moiety that occupies the BC Loop region of the bromodomain of CBP/p300;
R^{32a} is a moiety that occupies the lipophilic shelf (LPF) region of the bromodomain of CBP/p300;
each R³⁵ is independently hydrogen, C₁-C₄alkyl, or C₁-C₄fluoroalkyl;
m is 0, 1, 2, 3, or 4;
L is an optional linker;
wherein L is covalently attached to the R³² group, or at the position occupied by R³², or the R^{32a} group;
B-AR is a binder of the androgen receptor (AR), wherein the B-AR comprises:
   a head group that occupies the ligand-binding domain (LBD) of AR and is covalently attached to a core with a tail moiety covalently attached to the core moiety; wherein B-AR has the structure of Formula (D):

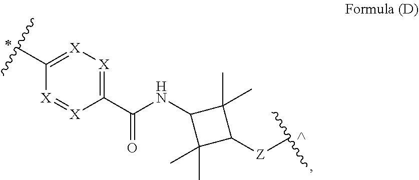

wherein head group is

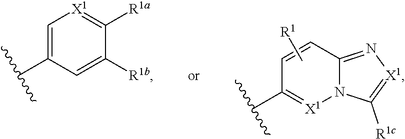

and the the head group is covalently attached to Z at position (\hat{ }), and L is attached at position *;
   Z is —O—, —NH— or —N(C₁-C₄alkyl)-;
   each X¹ is independently —CR¹— or —N—;
   R^{1a} is —CN, —NO₂, —C(═O)R⁵, —C(═O)OR⁵, or —C(═O)N(R⁵)₂;
   R^{1b} is hydrogen, halogen, substituted or unsubstituted C₁-C₄alkyl, C₁-C₄fluoroalkyl, —CN, —OH, or —OR⁴;
   R^{1c} is hydrogen, halogen, C₁-C₄alkyl, C₁-C₄fluoroalkyl, or —CN;
   each R¹ is independently hydrogen, halogen, substituted or unsubstituted C₁-C₄alkyl, C₁-C₄fluoroalkyl, —CN, —OH, or —OR⁴;
   each X is independently —CR³— or —N—;
   each R³ is independently hydrogen, halogen, substituted or unsubstituted C₁-C₄alkyl, C₁-C₄fluoroalkyl, —CN, —OH, —OR⁴, or —N(R⁵)₂.
   each R⁴ is independently substituted or unsubstituted C₁-C₄alkyl, or C₁-C₄fluoroalkyl;
   each R⁵ is independently hydrogen, substituted or unsubstituted C₁-C₄alkyl, C₁-C₄fluoroalkyl; and
   each s is 1, 2, or 3.

Embodiment 74. The compound of embodiment 73, or a pharmaceutically acceptable salt or solvate thereof, wherein:
R³² is hydrogen, substituted or unsubstituted C₁-C₆alkyl, substituted or unsubstituted C₃-C₁₂cycloalkyl, or substituted or unsubstituted 3- to 12-membered heterocycloalkyl;
R^{32a} is

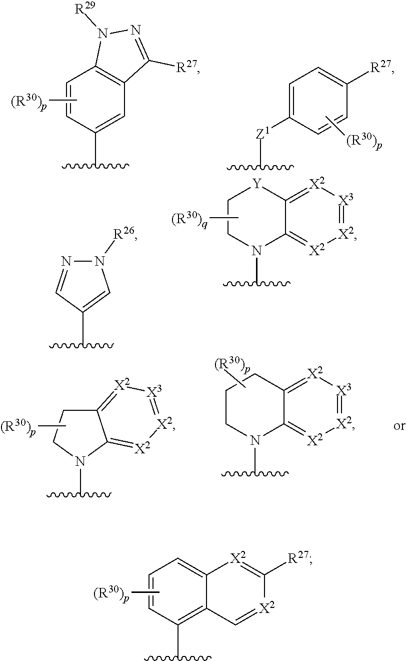

at least one X² is —CR³⁰— and at most two X² are —N—;
Z¹ is —NR^{c}— or —O—;
R^{c} is hydrogen or C₁-C₆alkyl;
R²⁶ is hydrogen, substituted or unsubstituted C₁-C₄alkyl, C₁-C₄fluoroalkyl, substituted or unsubstituted C₃-C₈cycloalkyl, or substituted or unsubstituted 3- to 8-membered heterocycloalkyl;
X³ is —CR²⁷— or —N—;
R²⁷ is hydrogen, halogen, substituted or unsubstituted C₁-C₄alkyl, C₁-C₄fluoroalkyl, substituted or unsubstituted C₃-C₈ cycloalkyl, substituted or unsubstituted 3- to 8-membered heterocycloalkyl, substituted or unsubstituted phenyl, substituted or unsubstituted monocyclic heteroaryl, —CN, —OH, —OR^{a}, —N(R^{b})₂, —NR^{b}C(═O)R^{a}, —C(═O)R^{b}, —C(═O)OR^{b}, or —C(═O)N(R^{b})₂;
or R²⁷ is

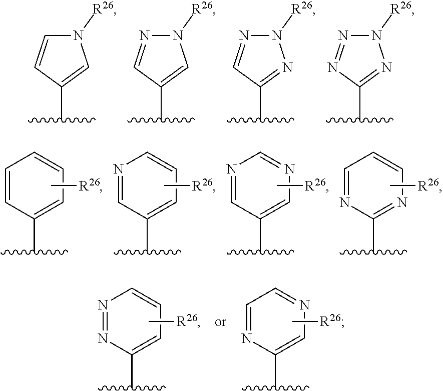

p is 0, 1, 2, or 3;
R²⁹ is hydrogen, substituted or unsubstituted C₁-C₄alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Y is —C(R³⁰)₂— or N(R²⁸)—;
each R³⁰ is independently hydrogen, halogen, C₁-C₄alkyl, C₁-C₄fluoroalkyl, substituted or unsubstituted C₁-C₆heteroalkyl, —CN, —OH, or —OR^{a};
q is 0, 1, 2, 3, or 4;
each R^{a} is independently C₁-C₄alkyl, C₁-C₆fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
each R^{b} is independently hydrogen, C₁-C₄alkyl, C₁-C₄fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
or two R^{b} on the same N atom are taken together with the N atom to which they are attached to form a substituted or unsubstituted N-containing heterocycle.

Embodiment 75. The compound of any one of embodiments 73-74, or a pharmaceutically acceptable salt or solvate thereof, wherein:
R²⁷ is hydrogen, halogen, C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic 5- or 6-membered heteroaryl, —CN, —OH, —OR^{a}, —N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
or R²⁷ is

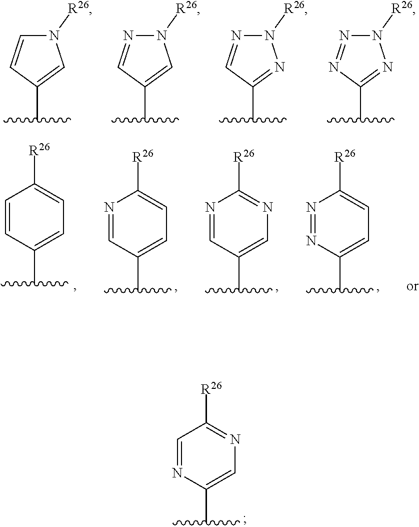

Embodiment 76. The compound of any one of embodiments 73-75, or a pharmaceutically acceptable salt or solvate thereof, wherein:
R³² is

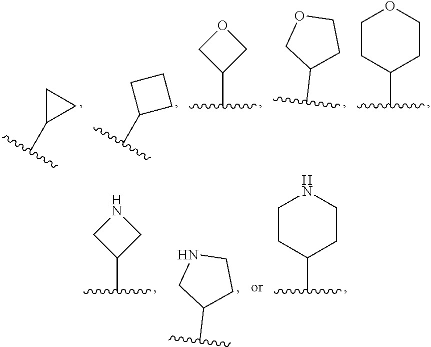

each of which is unsubstituted or substituted with F, Cl, Br, —CH₃, —CD₃, —CH₂CH₃, —CH₂F, —CHF₂, —CF₃, —CH₂CH₂F, —CH₂CHF₂, —CH₂CF₃, —CN, —C(═O)CH₃, —C(═O)CH₂CH₃, —C(═O)CH₂F, —C(═O)CHF₂, —C(═O)CF₃, —C(═O)CH₂CH₂F, —C(═O)CH₂CHF₂, —C(═O)CH₂CF₃, —C(═O)CD₃, —SO₂CH₃, —SO₂CH₂CH₃, —SO₂CD₃, or —SO₂CH₂CD₃.
R^{32a} is

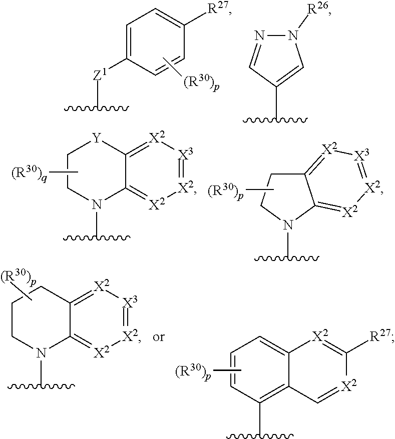

at least one X² is —CR³⁰— and at most two X² are —N—;
each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted aryl, substituted or unsubstituted heteroaryl, —CN, —OH, or —OR^{a}; and
each R^{a} is independently substituted or unsubstituted C₁-C₆alkyl.

Embodiment 77. The compound of any one of embodiments 73-76, or a pharmaceutically acceptable salt or solvate thereof, wherein:
R²⁷ is hydrogen, —CH₃, —CH₂CH₃, —CH₂CH₂CH₃, —CH₂(CH₃)₂, —(CH₃)₃, —F, —CHF₂, —CF₃, —CN, —OH, —OCH₃, cyclopropyl, cyclobutyl, oxetanyl, tetrahydrofuranyl, tetrahydropyranyl, unsubstituted or substituted phenyl, unsubstituted or substituted pyrazolyl, unsubstituted or substituted pyridinyl, or unsubstituted or substituted pyrimidinyl; or
R²⁷ is;

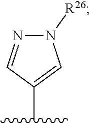

R³² is

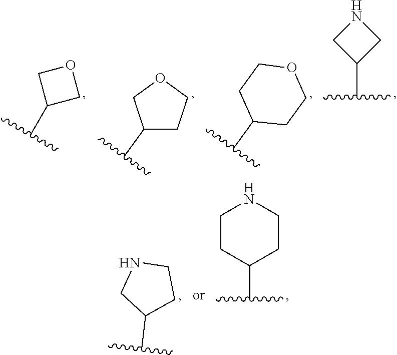 [SMILES: *C1COC1 ; *C1CCOC1 ; *C1CCOCC1 ; *C1CNC1 ; *C1CCNC1 ; *C1CCNCC1]

each of which is unsubstituted or substituted with F, Cl, Br, —CH₃, —CD₃, —CH₂CH₃, —CH₂F, —CHF₂, —CF₃, CN, —C(═O)CH₃, —C(═O)CH₂F, —C(═O)CHF₂, —C(═O)CF₃, or —C(═O)CD₃.

Embodiment 78. The compound of embodiment 73-77, or a pharmaceutically acceptable salt or solvate thereof, wherein:
R³² is

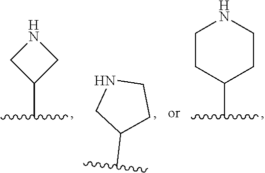 [SMILES: *C1CNC1 ; *C1CCNC1 ; *C1CCNCC1]

wherein the optional linker is covalently attached to the nitrogen of R³² group;
or R³² is absent and L is covalently attached to the acetyl-lysine mimetic moiety at the position occupied by R³²; and
R^{32a} is

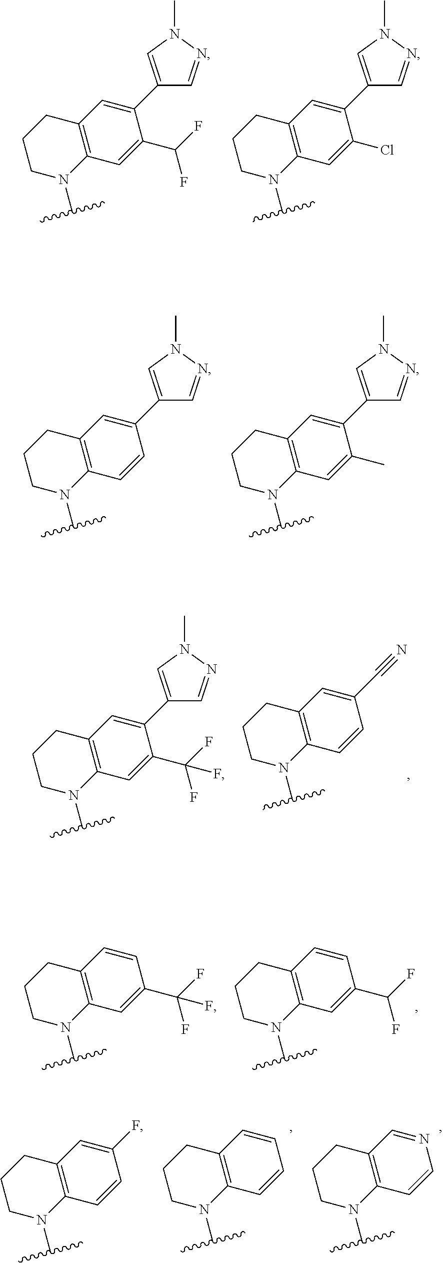

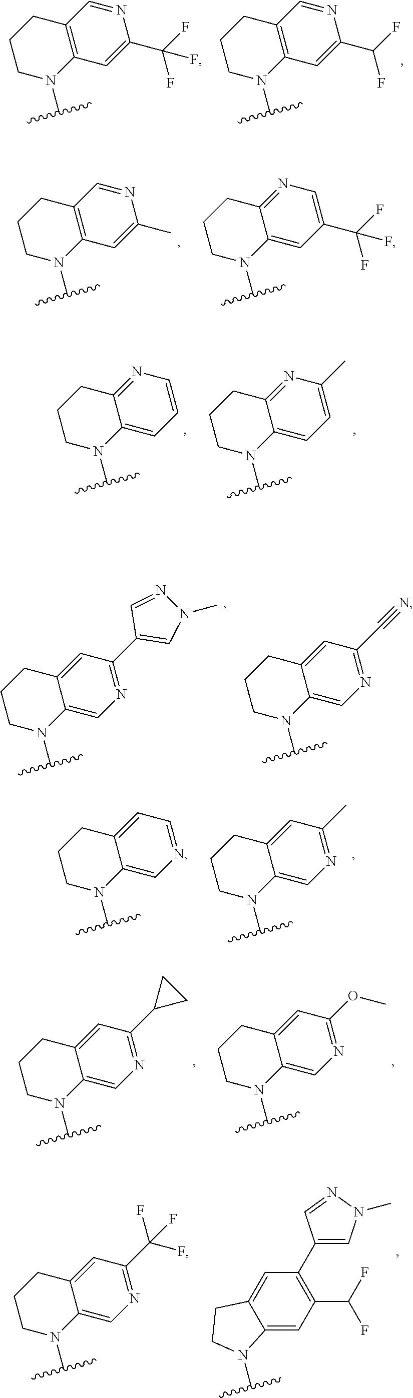

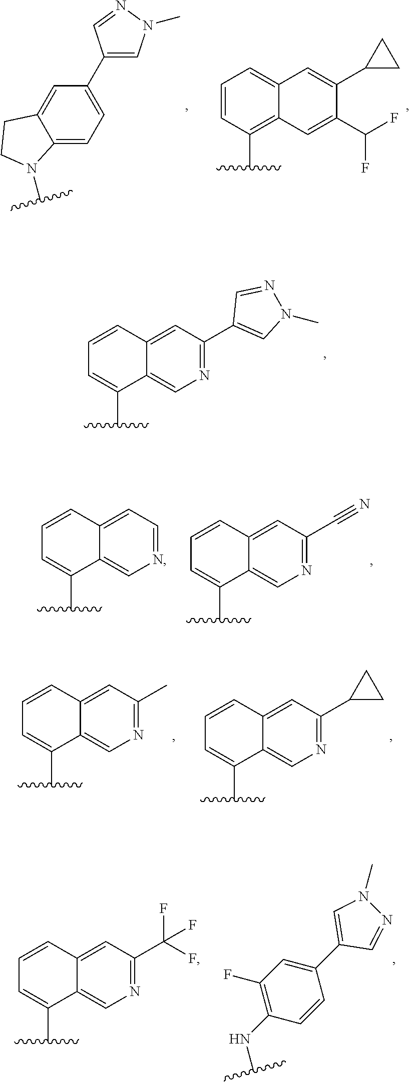

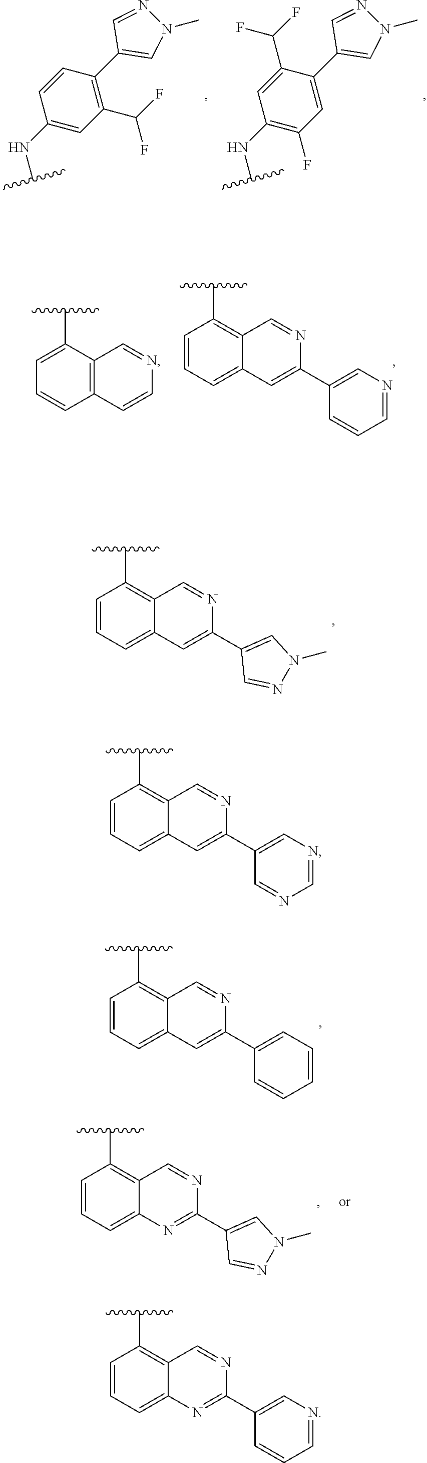

Embodiment 79. The compound of any one of embodiments 73-78, or a pharmaceutically acceptable salt or solvate thereof, wherein:
R³² is

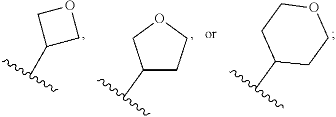 [SMILES: *C1COC1 ; *C1CCOC1 ; *C1CCOCC1]

R^{32a} is

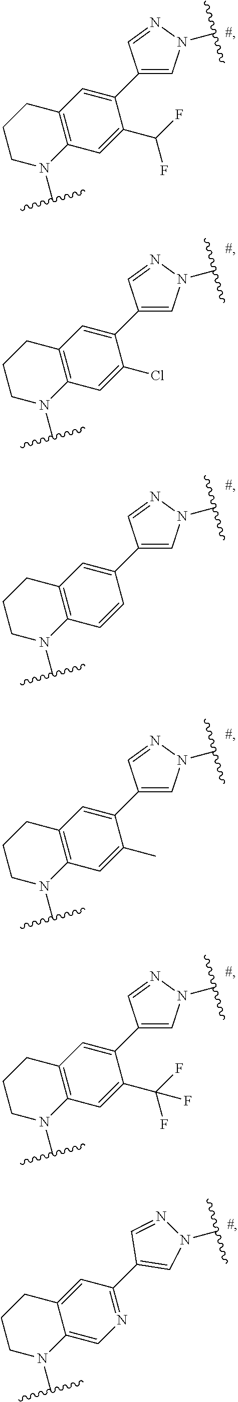 [SMILES: *N1CCCc2cc(-c3cnn(*)c3)c(C(F)F)cc21 ; *N1CCCc2cc(-c3cnn(*)c3)c(Cl)cc21 ; *N1CCCc2cc(-c3cnn(*)c3)ccc21 ; *N1CCCc2cc(-c3cnn(*)c3)c(C)cc21 ; *N1CCCc2cc(-c3cnn(*)c3)c(C(F)(F)F)cc21 ; *N1CCCc2cc(-c3cnn(*)c3)ncc21]

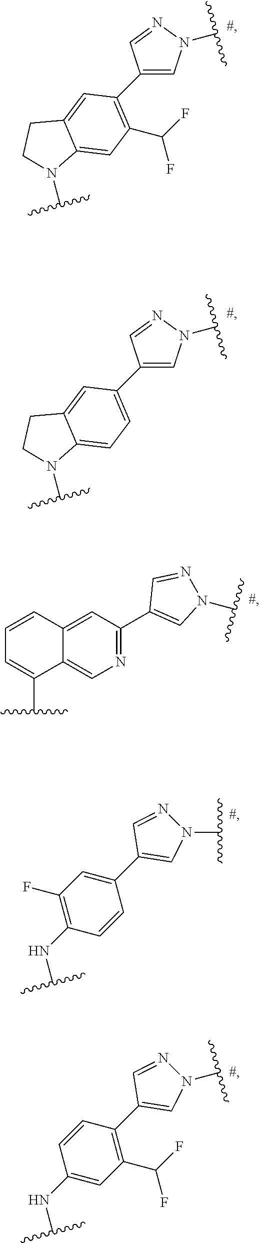 [SMILES: *N1CCc2cc(-c3cnn(*)c3)c(C(F)F)cc21 ; *N1CCc2cc(-c3cnn(*)c3)ccc21 ; *c1cccc2cc(-c3cnn(*)c3)ncc12 ; *Nc1ccc(-c2cnn(*)c2)cc1F ; *Nc1ccc(-c2cnn(*)c2)c(C(F)F)c1]

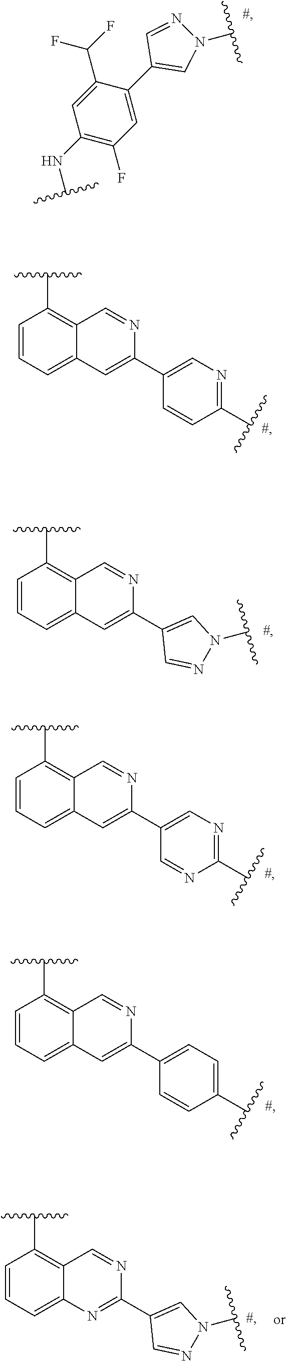 [SMILES: *Nc1cc(C(F)F)c(-c2cnn(*)c2)cc1F ; *c1ccc(-c2cc3cccc(*)c3cn2)cn1 ; *c1cccc2cc(-c3cnn(*)c3)ncc12 ; *c1ncc(-c2cc3cccc(*)c3cn2)cn1 ; *c1ccc(-c2cc3cccc(*)c3cn2)cc1 ; *c1cccc2nc(-c3cnn(*)c3)ncc12]

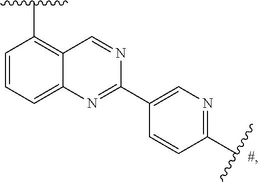 [SMILES: *c1ccc(-c2ncc3c(*)cccc3n2)cn1]

wherein

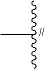

is the point of attachment to the optional linker.

Embodiment 80. The compound of embodiment 73, or a pharmaceutically acceptable salt or solvate thereof, wherein the head group is:

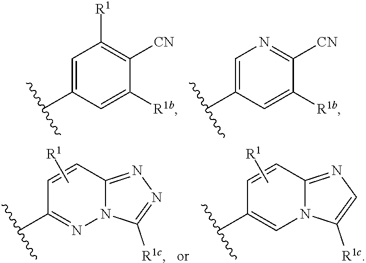

R^{1b} is hydrogen, F, Cl, Br, I, —CH₃, —CD₃, —CH₂CH₃, —CH₂F, —CHF₂, —CF₃, —OH, —OCF₃, —OCH₃, —OCH₂CH₃, —OCD₃, —OCH₂CD₃, or —CN;
each R^{1c} is hydrogen, F, Cl, Br, —CH₃, —CD₃, —CH₂F, —CHF₂, or —CF₃;
each R¹ is independently hydrogen, F, Cl, Br, I, —CH₃, —CD₃, —CH₂CH₃, —CH₂F, —CHF₂, —CF₃, —CH₂CH₂F, —CH₂CHF₂, —CH₂CF₃, —OH, —OCF₃, —OCH₃, —OCD₃, or —OCH₂CH₃.

Embodiment 81. The compound of embodiment 73, or a pharmaceutically acceptable salt or solvate thereof, wherein the head group is selected from:

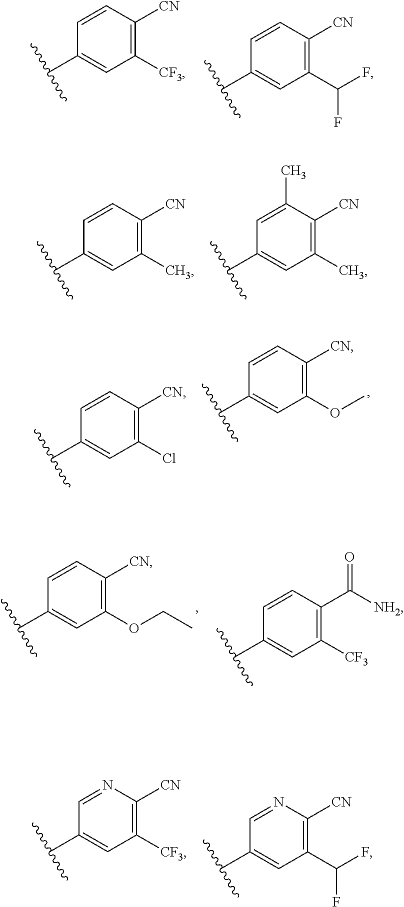

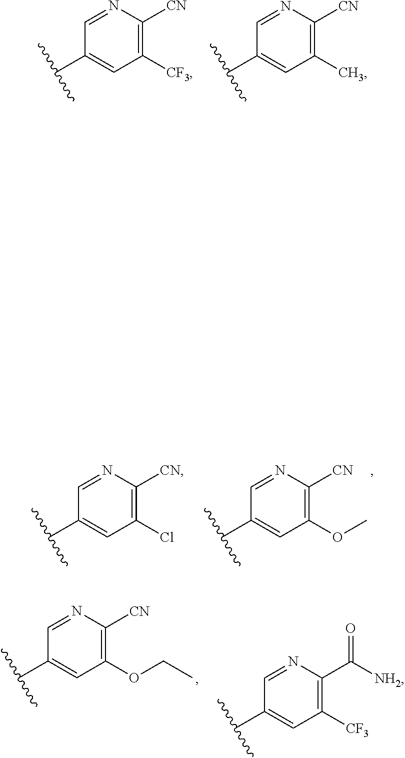 [SMILES: *c1cnc(C#N)c(C(F)(F)F)c1 ; *c1cnc(C#N)c(C)c1 ; *c1cnc(C#N)c(Cl)c1 ; *c1cnc(C#N)c(OC)c1 ; *c1cnc(C#N)c(OCC)c1 ; *c1cnc(C(N)=O)c(C(F)(F)F)c1]

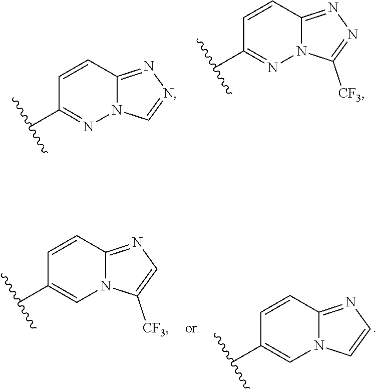 [SMILES: *c1ccc2nncn2n1 ; *c1ccc2nnc(C(F)(F)F)n2n1 ; *c1ccc2ncc(C(F)(F)F)n2c1 ; *c1ccc2nccn2c1]

Embodiment 82. The compound of any one of embodiments 14-81, or a pharmaceutically acceptable salt or solvate thereof, wherein:
SB-CBP/p300 has the structure:

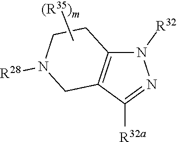

R²⁸ is —C(═O)CH₃ or —C(═O)NH(CH₃);
each R³⁵ is independently hydrogen, —CH₃—CH₂F, —CHF₂, —CF₃;
m is 0, 1, or 2
R³² is

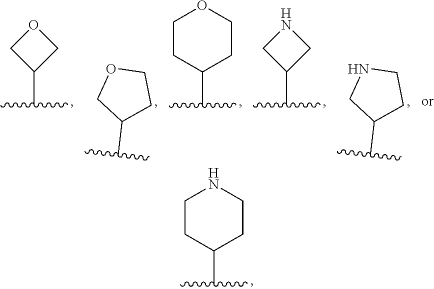

each of which is unsubstituted or substituted with F, —CH₃, —CH₂F, —CHF₂, or —CF₃;
or R³² is absent and the optional linker is covalently attached to the acetyl-lysine mimetic moiety at the position occupied by R².
R^{32a} is

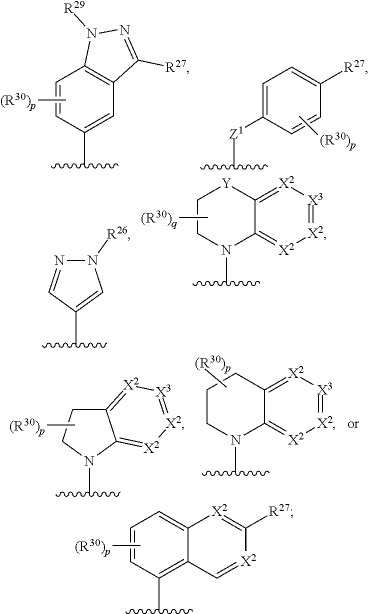

at least one X² is —CR³⁰— and at most two X² are N—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is

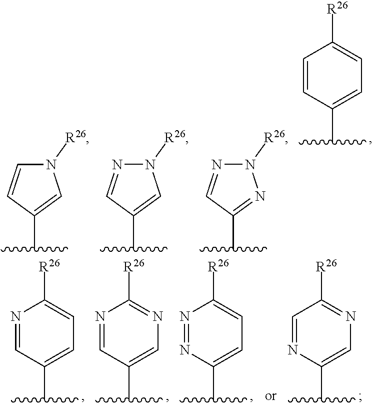

each R³⁰ is independently hydrogen, halogen, C₁-C₄alkyl, C₁-C₄fluoroalkyl, —CN, —OH, or —OR^{a}, R^{a} is C₁-C₄alkyl;
each R^{b} is independently hydrogen or C₁-C₆alkyl;
wherein L is covalently attached to the R³² group, or at the position occupied by R²⁶, or at the position occupied by R²⁷;

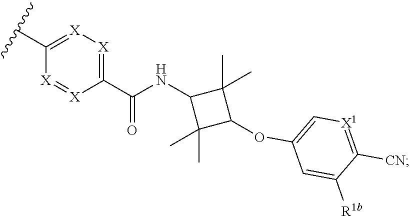

B-AR has the structure: R^{1b}
each X is independently —CR³— or —N—;
X¹ is —CR¹— or —N—;
R^{1b} is hydrogen, F, Cl, —CH₃, —CH₂F, —CHF₂, —CF₃, or —OCF₃;
R¹ is hydrogen, F, Cl, —CH₃, —CH₂F, —CHF₂, or —CF₃;

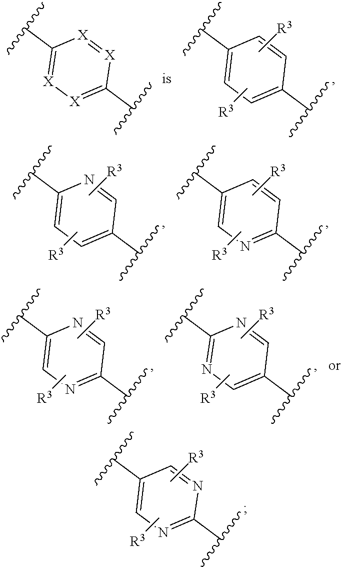

each R³ is independently hydrogen, F, Cl, Br, —CH₃, —CH₂F, —CHF₂, —CF₃, —OH, —OCF₃, —OCH₃, or —CN.

Embodiment 83. The compound of embodiment 82, or a pharmaceutically acceptable salt or solvate thereof, wherein:

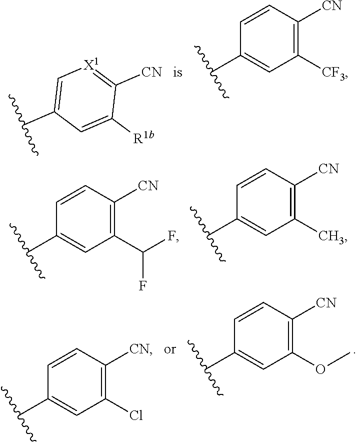

Embodiment 84. The compound of any one of embodiments 82-83, or a pharmaceutically acceptable salt or solvate thereof, wherein:
R³² is

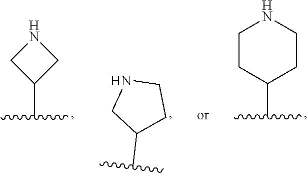 [SMILES: *C1CNC1 ; *C1CCNC1 ; *C1CCNCC1]

wherein the optional linker is covalently attached to the nitrogen of R³² group;
or R³² is absent and the optional linker is covalently attached to the acetyl-lysine mimetic moiety at the position occupied by R³²;
R^{32a} is

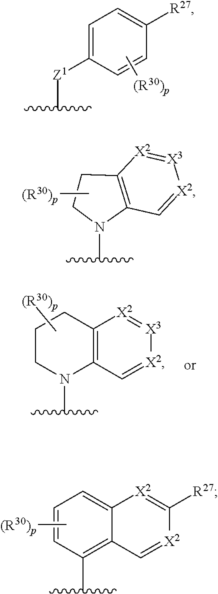

at least one X² is —CR³⁰— and at most two X² are —N—;
X3 is —CR²⁷— or —N—;
Z¹ is —NH— or —O—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is

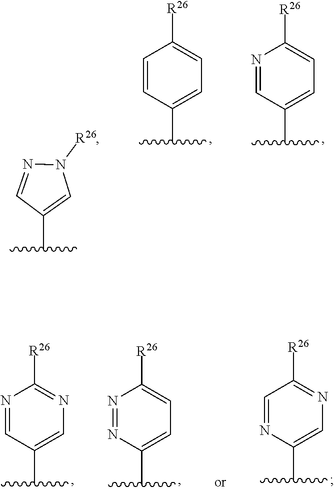

and
each R³⁰ is independently hydrogen, halogen, C₁-C₄alkyl, C₁-C₄fluoroalkyl, —CN, —OH, or —OR^{a}.

Embodiment 85. The compound of any one of embodiments 82-84, or a pharmaceutically acceptable salt or solvate thereof, wherein:
R³² is

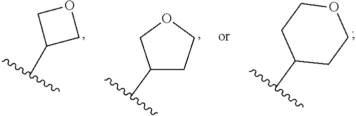 [SMILES: *C1COC1 ; *C1CCOC1 ; *C1CCOCC1]

R^{32a} is

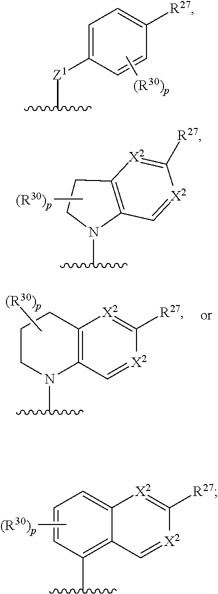

R²⁷ is

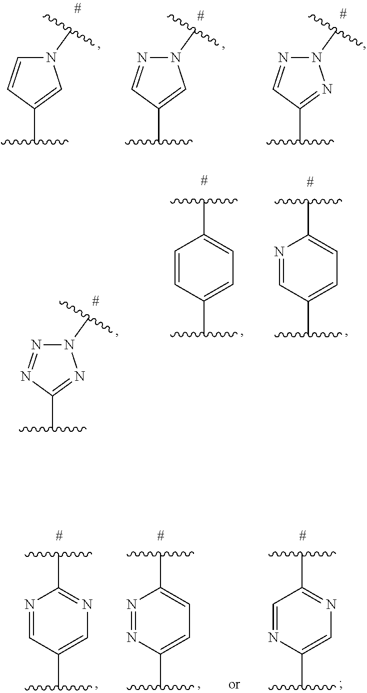

Z¹ is —NH— or —O—;

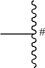

is the point of attachment to the optional linker.

Embodiment 86. The compound of any one of embodiments 14-81, or a pharmaceutically acceptable salt or solvate thereof, wherein SB-CBP/p300 has the structure of Formula (IIIb):

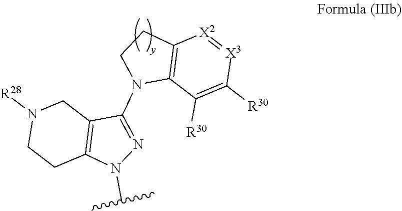

wherein:
X² is —CR³⁰— or —N—;
X³ is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
each R³⁰ is independently hydrogen, halogen, C₁-C₄alkyl, C₁-C₄fluoroalkyl, —CN, —OH, or —OR^{a};
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 87. The compound of any one of embodiments 14-81, or a pharmaceutically acceptable salt or solvate thereof, wherein SB-CBP/p300 has the structure of Formula (IIIc):

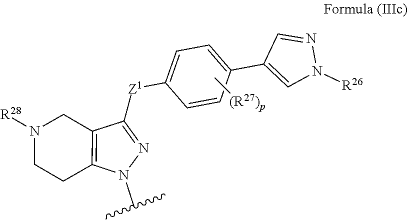

wherein:
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z¹ is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 88. The compound of any one of embodiments 14-81, or a pharmaceutically acceptable salt or solvate thereof, wherein SB-CBP/p300 has the structure of Formula (IIId-1) or (IIId-2):

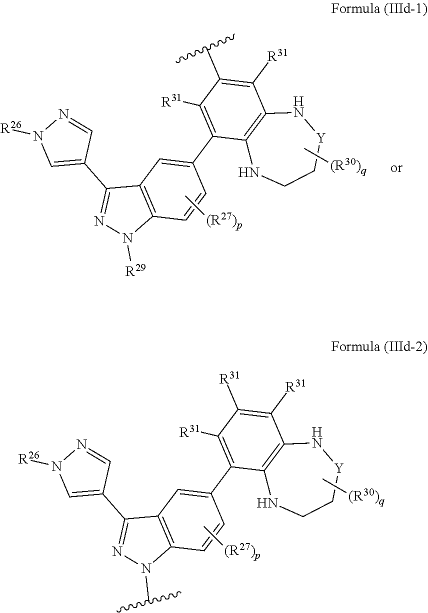

wherein:
R²⁶ is hydrogen, substituted or unsubstituted C₁-C₆alkyl, substituted or unsubstituted C₁-C₆heteroalkyl, substituted or unsubstituted C₃-C₈ cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂- or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 89. The compound of any one of embodiments 14-81, or a pharmaceutically acceptable salt or solvate thereof, wherein SB-CBP/p300 has the structure of Formula (IIIe):

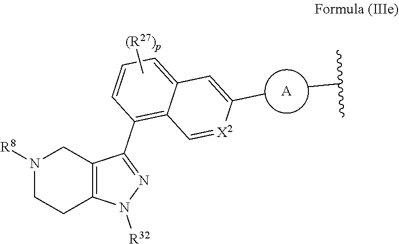

wherein:
X² is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Ring A is absent or substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 90. The compound of any one of embodiments 14-89, or a pharmaceutically acceptable salt or solvate thereof, wherein SB-CBP/p300 has one of the following structures:

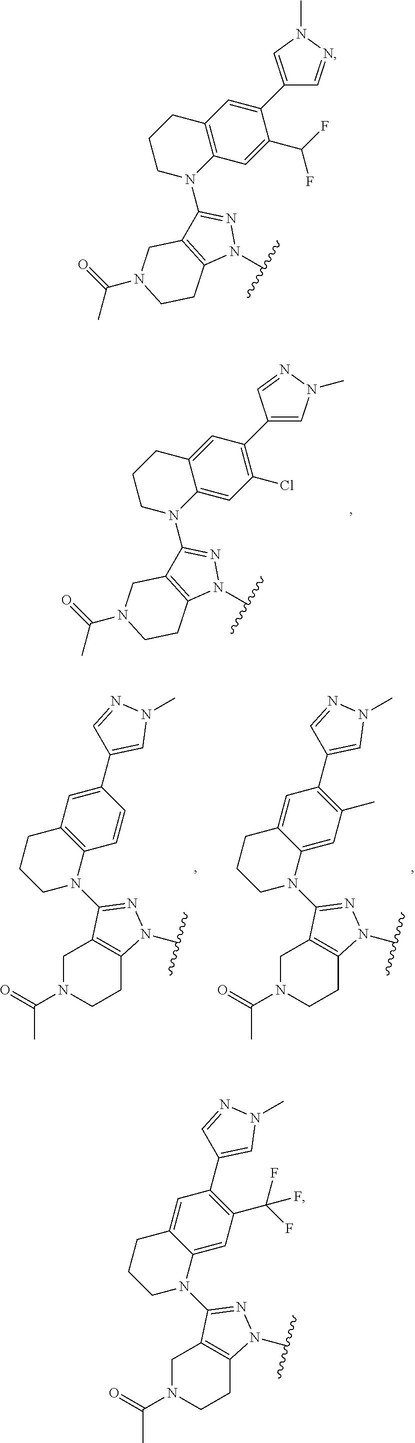 [SMILES: *n1nc(N2CCCc3cc(-c4cnn(C)c4)c(C(F)F)cc32)c2c1CCN(C(C)=O)C2 ; *n1nc(N2CCCc3cc(-c4cnn(C)c4)c(Cl)cc32)c2c1CCN(C(C)=O)C2 ; *n1nc(N2CCCc3cc(-c4cnn(C)c4)ccc32)c2c1CCN(C(C)=O)C2 ; *n1nc(N2CCCc3cc(-c4cnn(C)c4)c(C)cc32)c2c1CCN(C(C)=O)C2 ; *n1nc(N2CCCc3cc(-c4cnn(C)c4)c(C(F)(F)F)cc32)c2c1CCN(C(C)=O)C2]

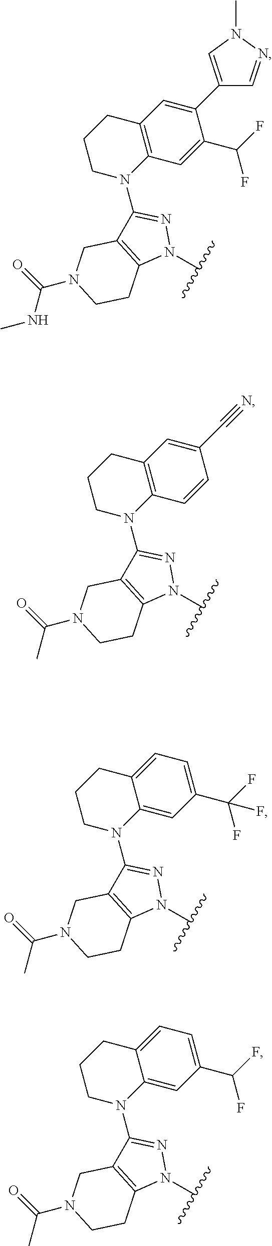 [SMILES: *n1nc(N2CCCc3cc(-c4cnn(C)c4)c(C(F)F)cc32)c2c1CCN(C(=O)NC)C2 ; *n1nc(N2CCCc3cc(C#N)ccc32)c2c1CCN(C(C)=O)C2 ; *n1nc(N2CCCc3ccc(C(F)(F)F)cc32)c2c1CCN(C(C)=O)C2 ; *n1nc(N2CCCc3ccc(C(F)F)cc32)c2c1CCN(C(C)=O)C2]

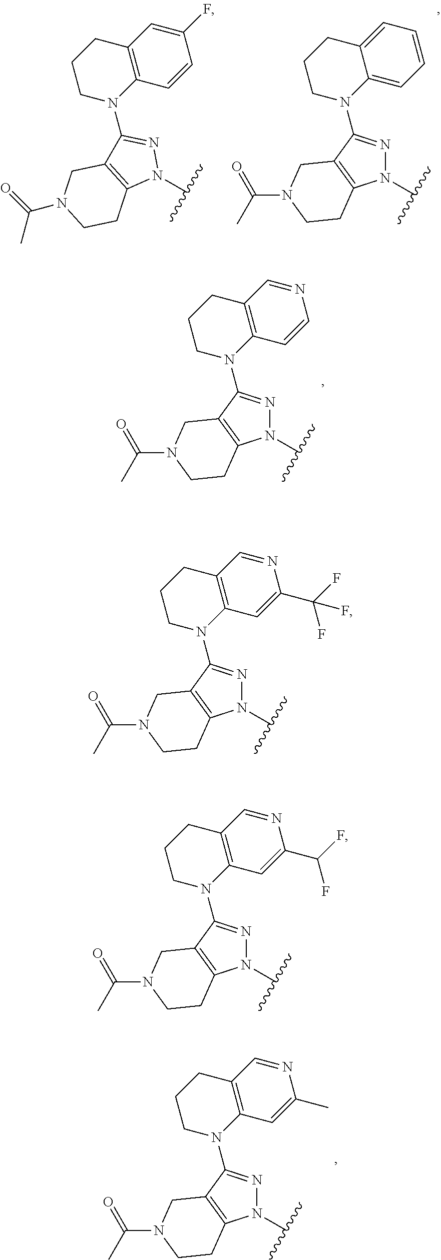 [SMILES: *n1nc(N2CCCc3cc(F)ccc32)c2c1CCN(C(C)=O)C2 ; *n1nc(N2CCCc3ccccc32)c2c1CCN(C(C)=O)C2 ; *n1nc(N2CCCc3cnccc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3cnc(C)cc32)c2c1CCN(C(C)=O)C2]

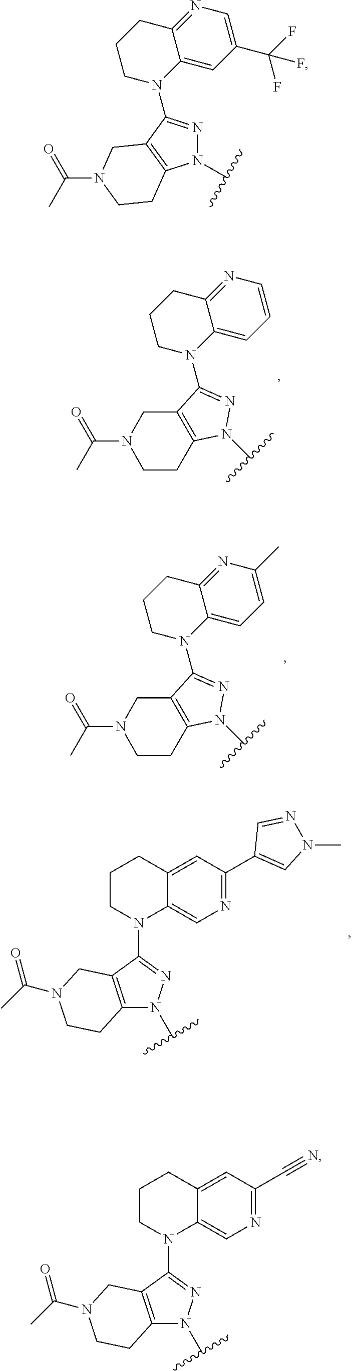

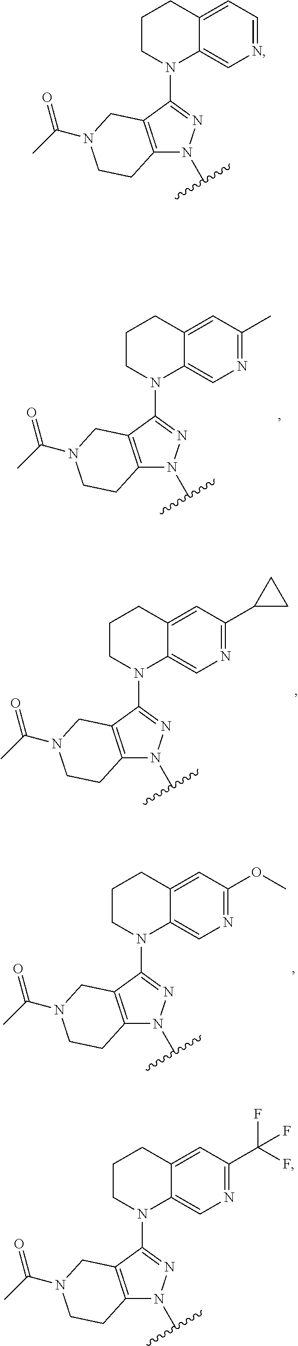

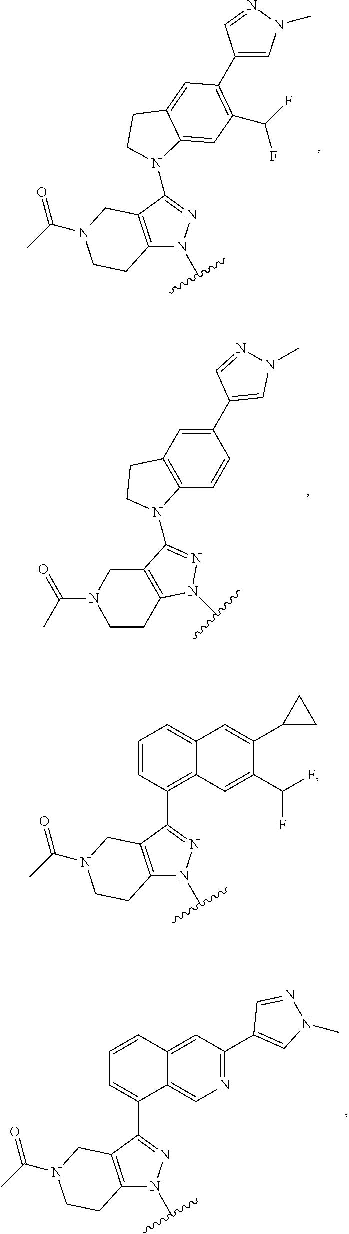

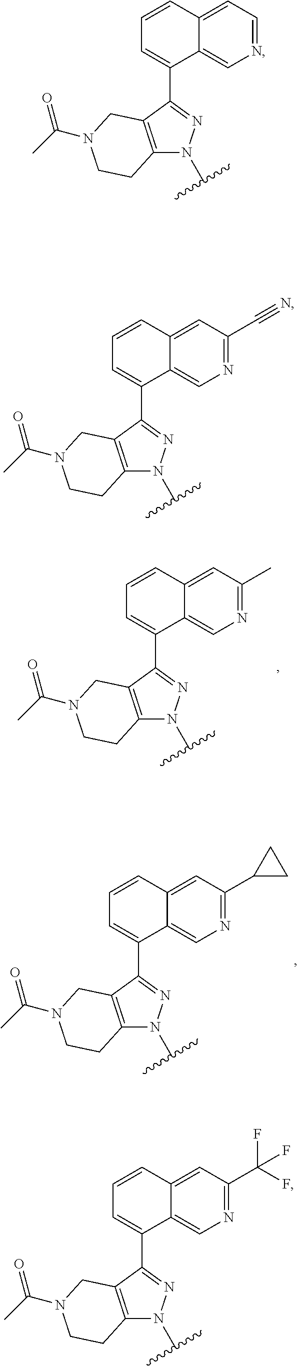

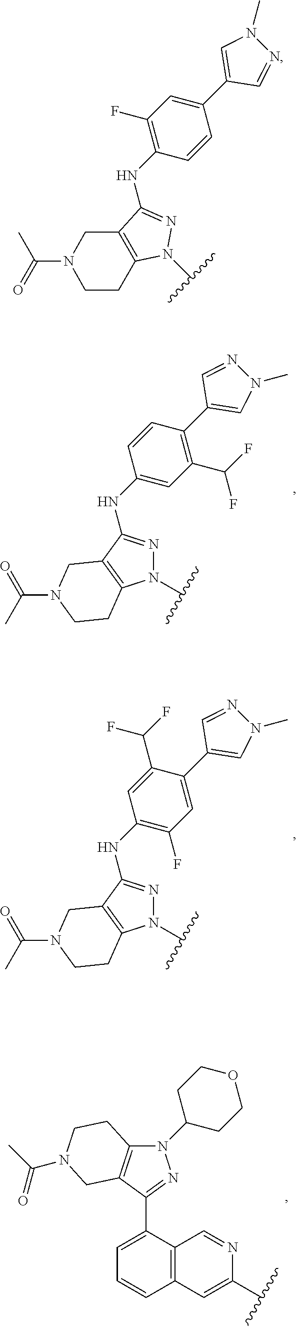

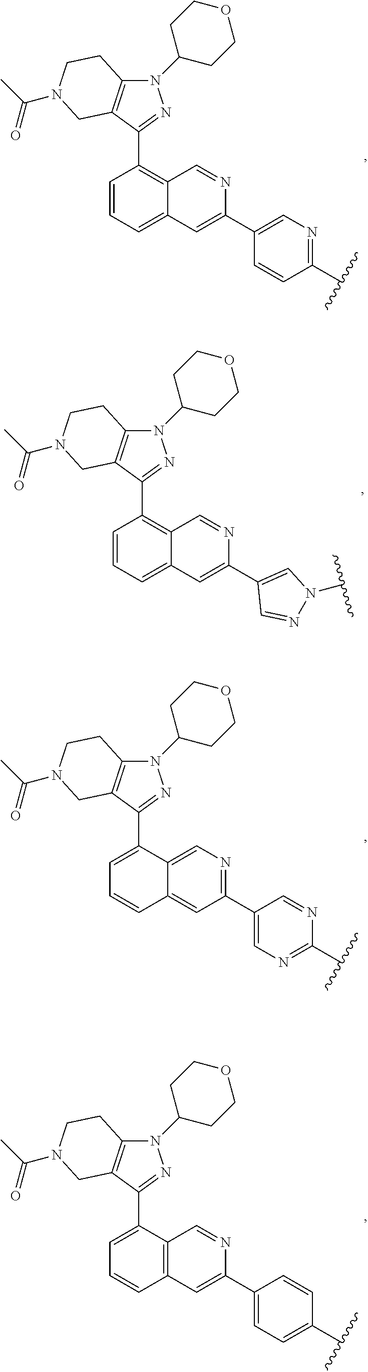 [SMILES: *c1ccc(-c2cc3cccc(-c4nn(C5CCOCC5)c5c4CN(C(C)=O)CC5)c3cn2)cn1 ; *n1cc(-c2cc3cccc(-c4nn(C5CCOCC5)c5c4CN(C(C)=O)CC5)c3cn2)cn1 ; *c1ncc(-c2cc3cccc(-c4nn(C5CCOCC5)c5c4CN(C(C)=O)CC5)c3cn2)cn1 ; *c1ccc(-c2cc3cccc(-c4nn(C5CCOCC5)c5c4CN(C(C)=O)CC5)c3cn2)cc1]

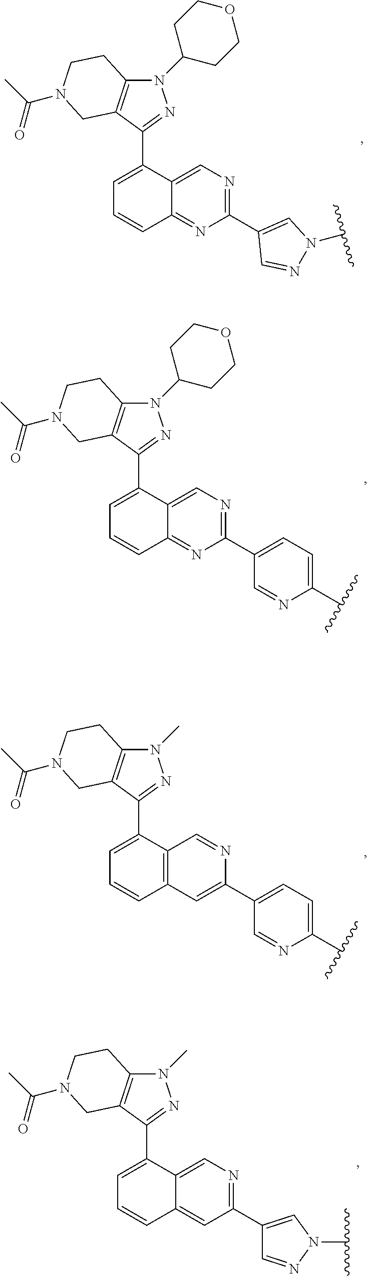 [SMILES: *n1cc(-c2ncc3c(-c4nn(C5CCOCC5)c5c4CN(C(C)=O)CC5)cccc3n2)cn1 ; *c1ccc(-c2ncc3c(-c4nn(C5CCOCC5)c5c4CN(C(C)=O)CC5)cccc3n2)cn1 ; *c1ccc(-c2cc3cccc(-c4nn(C)c5c4CN(C(C)=O)CC5)c3cn2)cn1 ; *n1cc(-c2cc3cccc(-c4nn(C)c5c4CN(C(C)=O)CC5)c3cn2)cn1]

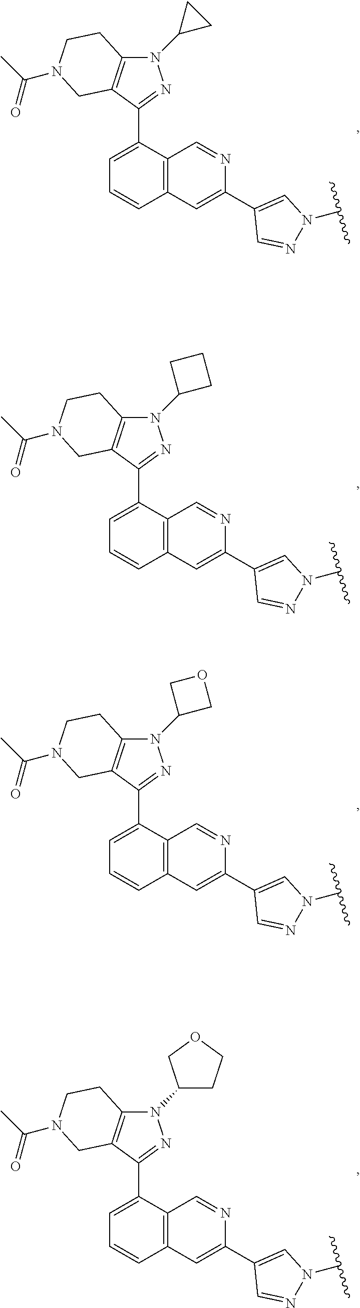 [SMILES: *n1cc(-c2cc3cccc(-c4nn(C5CC5)c5c4CN(C(C)=O)CC5)c3cn2)cn1 ; *n1cc(-c2cc3cccc(-c4nn(C5CCC5)c5c4CN(C(C)=O)CC5)c3cn2)cn1 ; *n1cc(-c2cc3cccc(-c4nn(C5COC5)c5c4CN(C(C)=O)CC5)c3cn2)cn1 ; *n1cc(-c2cc3cccc(-c4nn([C@H]5CCOC5)c5c4CN(C(C)=O)CC5)c3cn2)cn1]

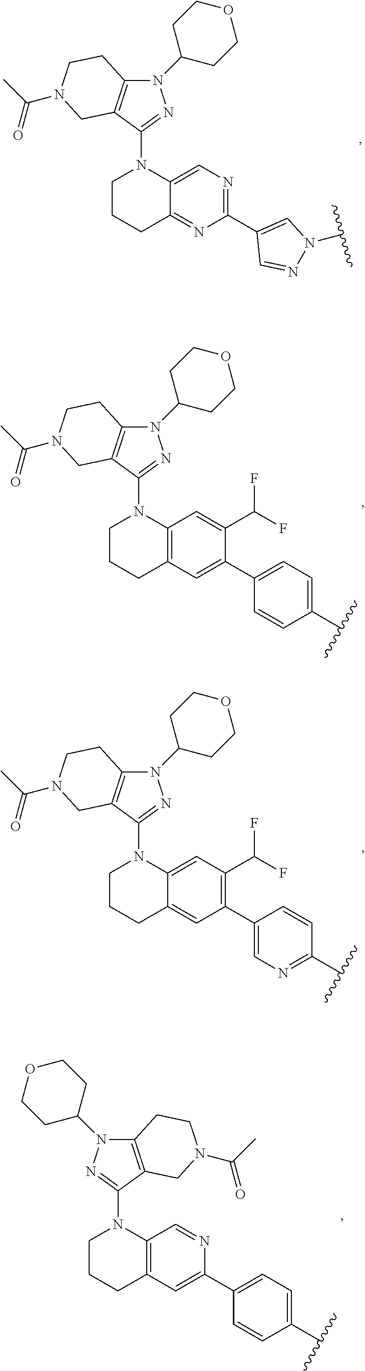 [SMILES: *n1cc(-c2ncc3c(n2)CCCN3c2nn(C3CCOCC3)c3c2CN(C(C)=O)CC3)cn1 ; *c1ccc(-c2cc3c(cc2C(F)F)N(c2nn(C4CCOCC4)c4c2CN(C(C)=O)CC4)CCC3)cc1 ; *c1ccc(-c2cc3c(cc2C(F)F)N(c2nn(C4CCOCC4)c4c2CN(C(C)=O)CC4)CCC3)cn1 ; *c1ccc(-c2cc3c(cn2)N(c2nn(C4CCOCC4)c4c2CN(C(C)=O)CC4)CCC3)cc1]

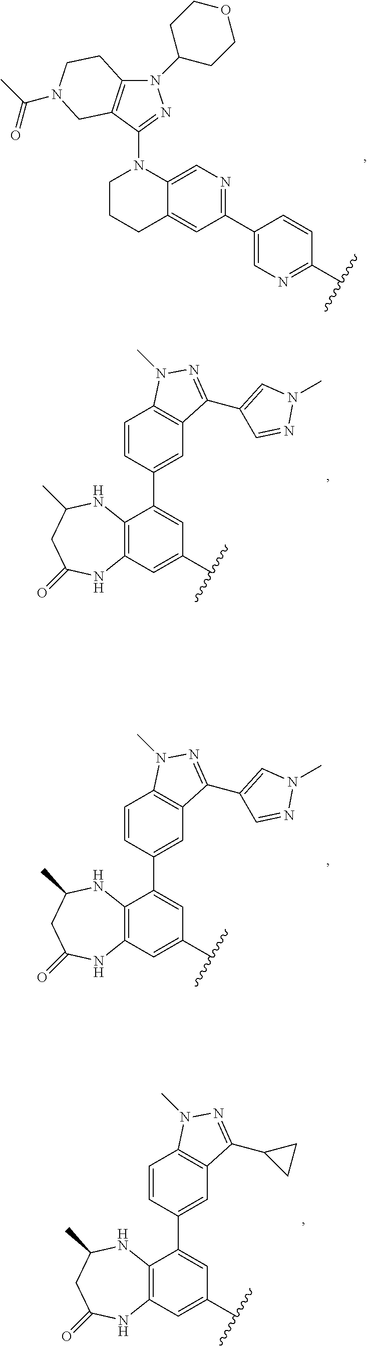 [SMILES: *c1ccc(-c2cc3c(cn2)N(c2nn(C4CCOCC4)c4c2CN(C(C)=O)CC4)CCC3)cn1 ; *c1cc2c(c(-c3ccc4c(c3)c(-c3cnn(C)c3)nn4C)c1)NC(C)CC(=O)N2 ; *c1cc2c(c(-c3ccc4c(c3)c(-c3cnn(C)c3)nn4C)c1)N[C@H](C)CC(=O)N2 ; *c1cc2c(c(-c3ccc4c(c3)c(C3CC3)nn4C)c1)N[C@H](C)CC(=O)N2]

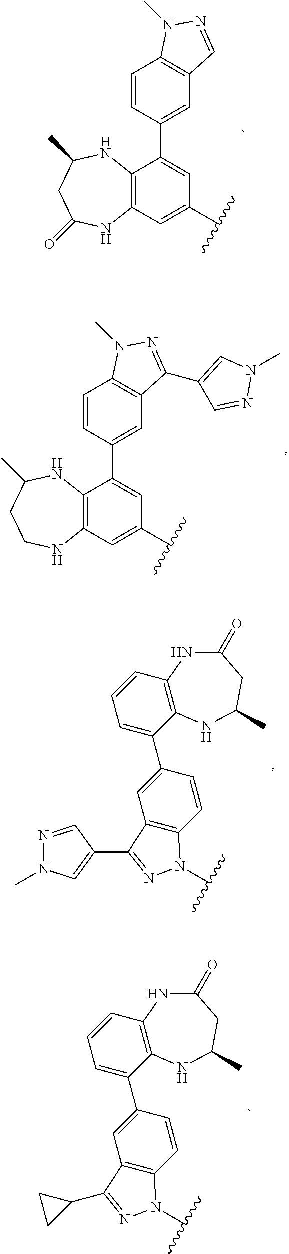

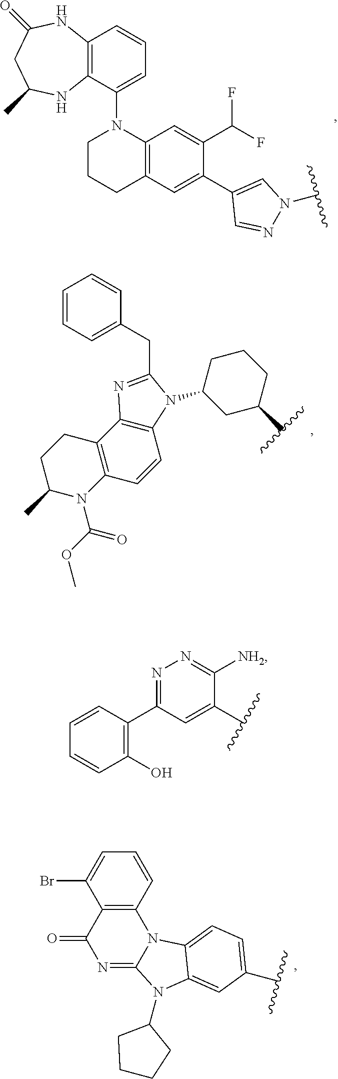 [SMILES: *n1cc(-c2cc3c(cc2C(F)F)N(c2cccc4c2N[C@@H](C)CC(=O)N4)CCC3)cn1 ; *[C@@H]1CCC[C@@H](n2c(Cc3ccccc3)nc3c4c(ccc32)N(C(=O)OC)[C@@H](C)CC4)C1 ; *c1cc(-c2ccccc2O)nnc1N ; *c1ccc2c(c1)n(C1CCCC1)c1nc(=O)c3c(Br)cccc3n21]

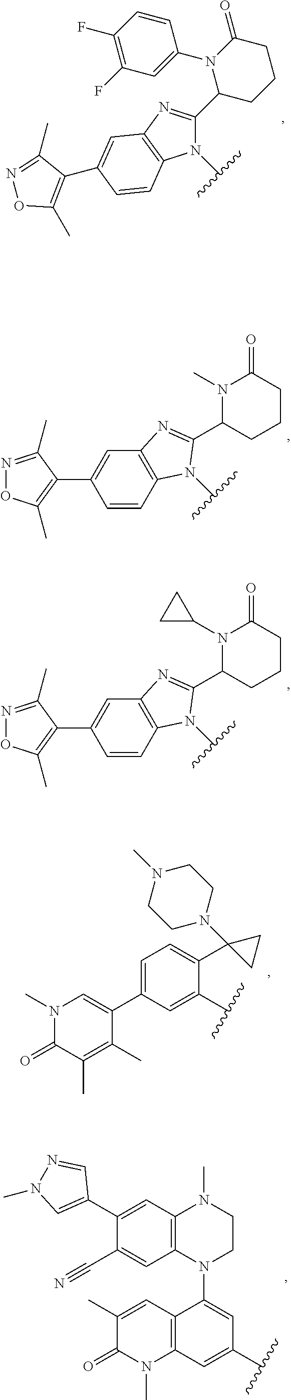 [SMILES: *n1c(C2CCCC(=O)N2c2ccc(F)c(F)c2)nc2cc(-c3c(C)noc3C)ccc21 ; *n1c(C2CCCC(=O)N2C)nc2cc(-c3c(C)noc3C)ccc21 ; *n1c(C2CCCC(=O)N2C2CC2)nc2cc(-c3c(C)noc3C)ccc21 ; *c1cc(-c2cn(C)c(=O)c(C)c2C)ccc1C1(N2CCN(C)CC2)CC1 ; *c1cc(N2CCN(C)c3cc(-c4cnn(C)c4)c(C#N)cc32)c2cc(C)c(=O)n(C)c2c1]

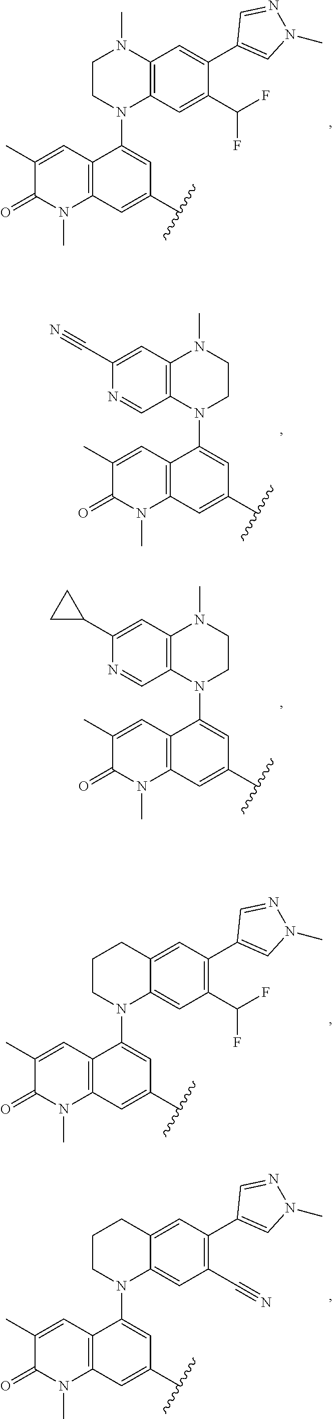 [SMILES: *c1cc(N2CCN(C)c3cc(-c4cnn(C)c4)c(C(F)F)cc32)c2cc(C)c(=O)n(C)c2c1 ; *c1cc(N2CCN(C)c3cc(C#N)ncc32)c2cc(C)c(=O)n(C)c2c1 ; *c1cc(N2CCN(C)c3cc(C4CC4)ncc32)c2cc(C)c(=O)n(C)c2c1 ; *c1cc(N2CCCc3cc(-c4cnn(C)c4)c(C(F)F)cc32)c2cc(C)c(=O)n(C)c2c1 ; *c1cc(N2CCCc3cc(-c4cnn(C)c4)c(C#N)cc32)c2cc(C)c(=O)n(C)c2c1]

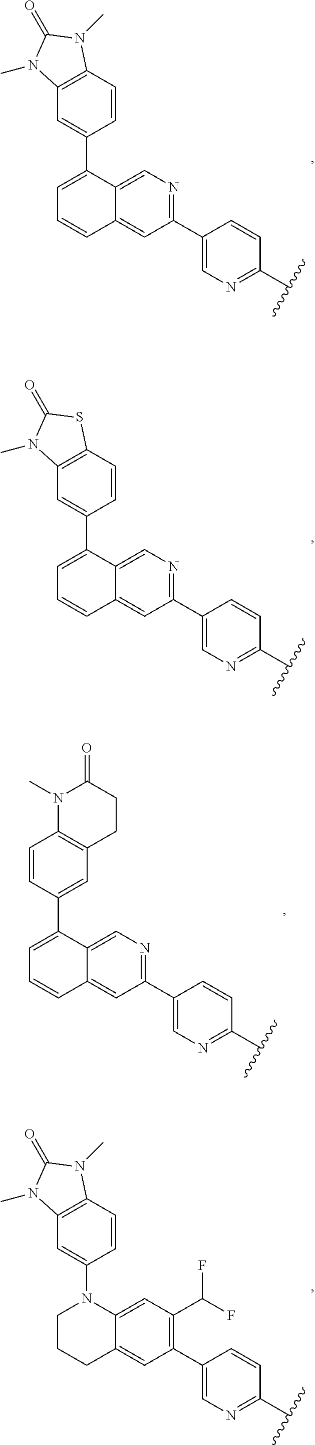 [SMILES: *c1ccc(-c2cc3cccc(-c4ccc5c(c4)n(C)c(=O)n5C)c3cn2)cn1 ; *c1ccc(-c2cc3cccc(-c4ccc5sc(=O)n(C)c5c4)c3cn2)cn1 ; *c1ccc(-c2cc3cccc(-c4ccc5c(c4)CCC(=O)N5C)c3cn2)cn1 ; *c1ccc(-c2cc3c(cc2C(F)F)N(c2ccc4c(c2)n(C)c(=O)n4C)CCC3)cn1]

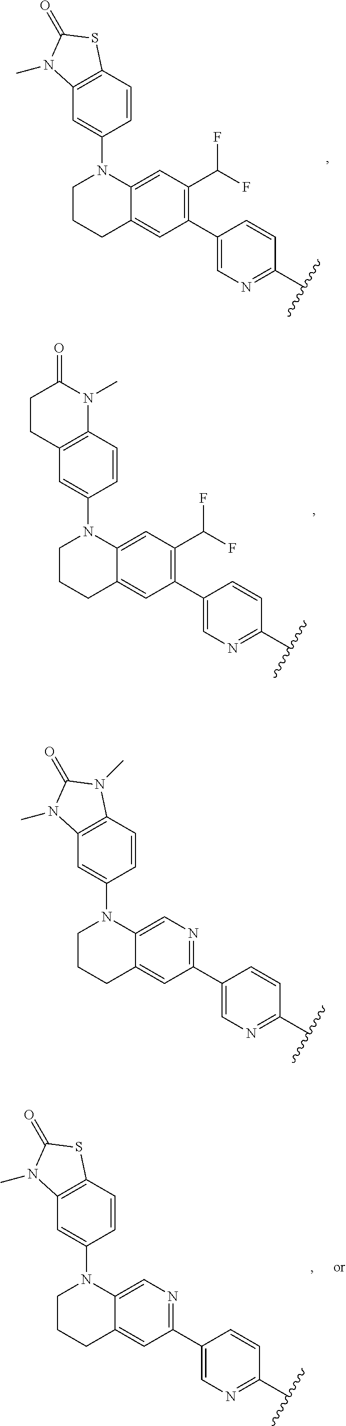 [SMILES: *c1ccc(-c2cc3c(cc2C(F)F)N(c2ccc4sc(=O)n(C)c4c2)CCC3)cn1 ; *c1ccc(-c2cc3c(cc2C(F)F)N(c2ccc4c(c2)CCC(=O)N4C)CCC3)cn1 ; *c1ccc(-c2cc3c(cn2)N(c2ccc4c(c2)n(C)c(=O)n4C)CCC3)cn1 ; *c1ccc(-c2cc3c(cn2)N(c2ccc4sc(=O)n(C)c4c2)CCC3)cn1]

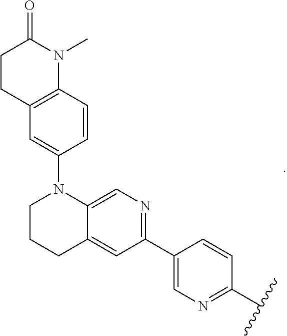 [SMILES: *c1ccc(-c2cc3c(cn2)N(c2ccc4c(c2)CCC(=O)N4C)CCC3)cn1]

Embodiment 91. The compound of anyone of embodiments 14-89, wherein SB-CBP/p300 has one of the following structures:

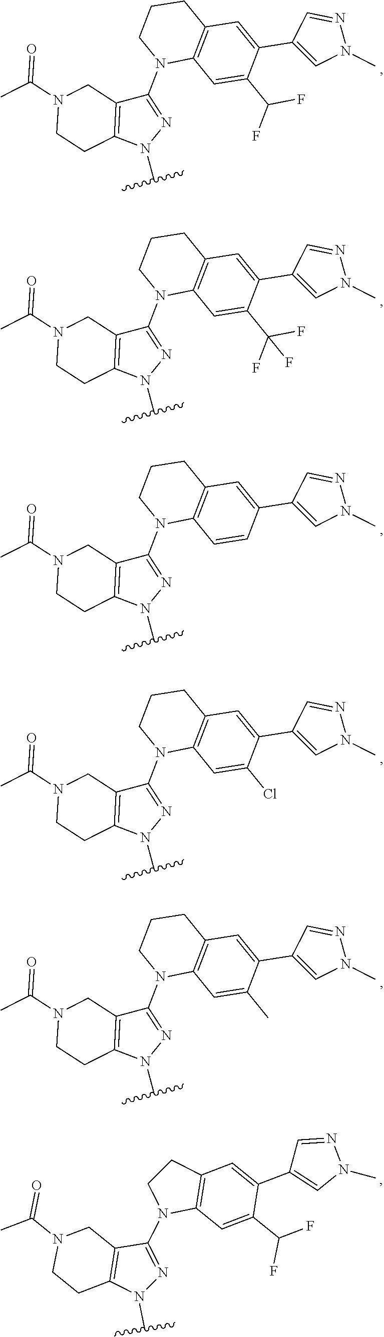

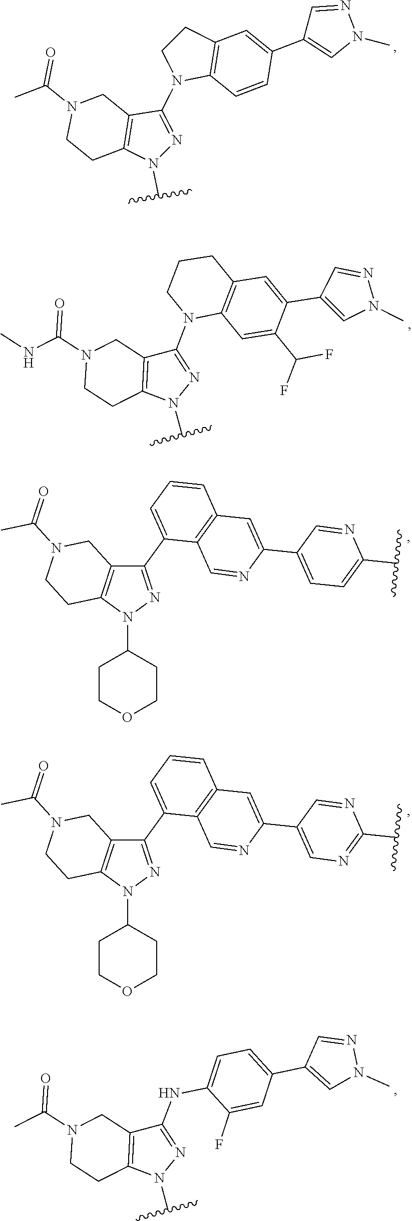

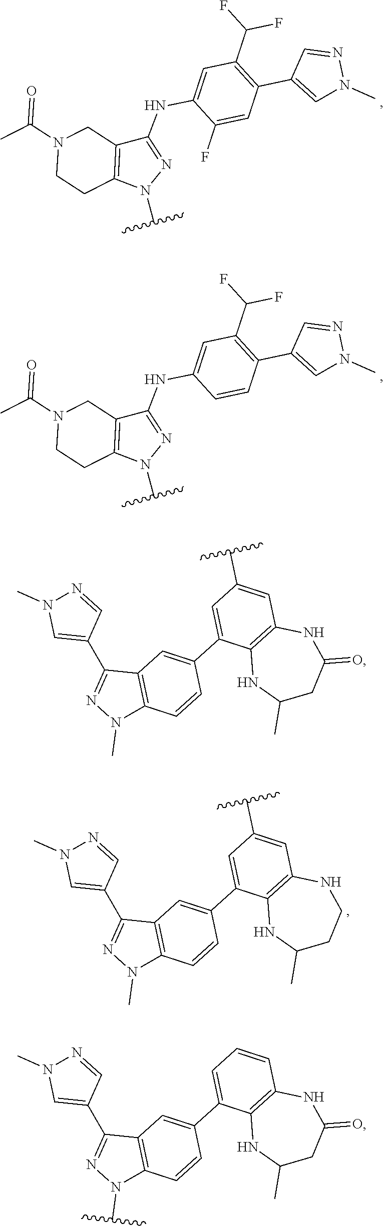

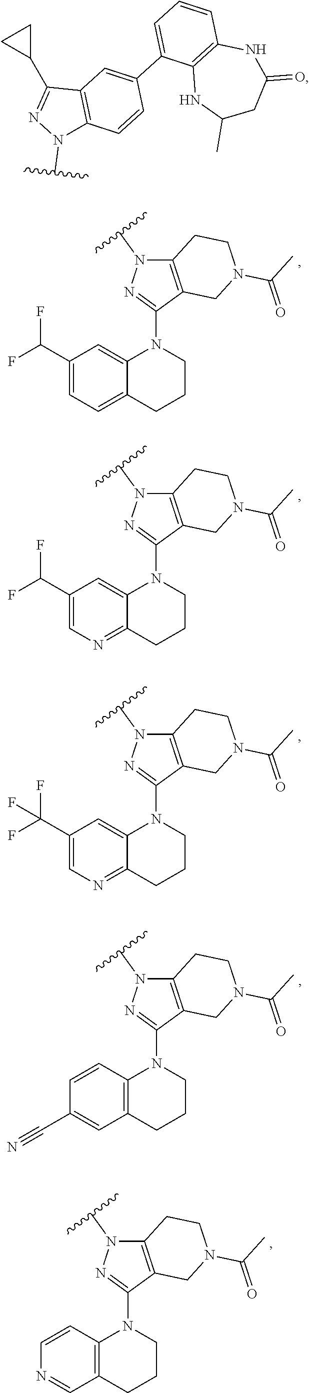 [SMILES: *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2 ; *n1nc(N2CCCc3cc(C#N)ccc32)c2c1CCN(C(C)=O)C2 ; *n1nc(N2CCCc3cnccc32)c2c1CCN(C(C)=O)C2]

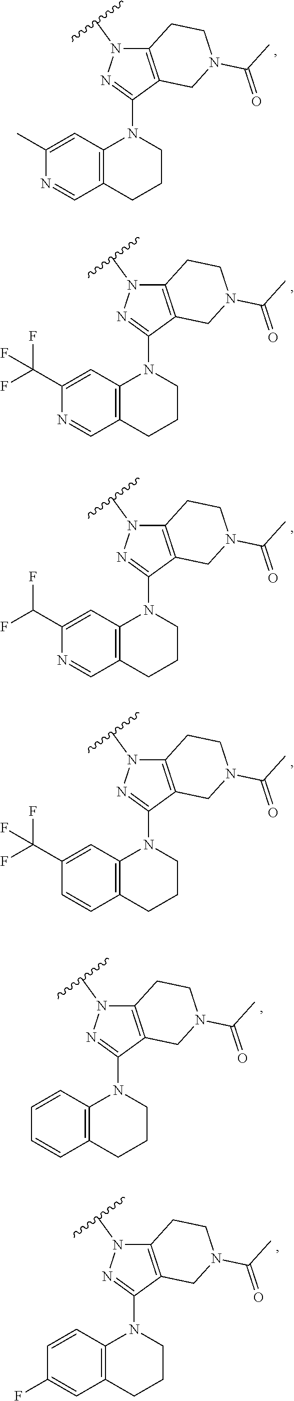 [SMILES: *n1nc(N2CCCc3cnc(C)cc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3ccc(C(F)(F)F)cc32)c2c1CCN(C(C)=O)C2 ; *n1nc(N2CCCc3ccccc32)c2c1CCN(C(C)=O)C2 ; *n1nc(N2CCCc3cc(F)ccc32)c2c1CCN(C(C)=O)C2]

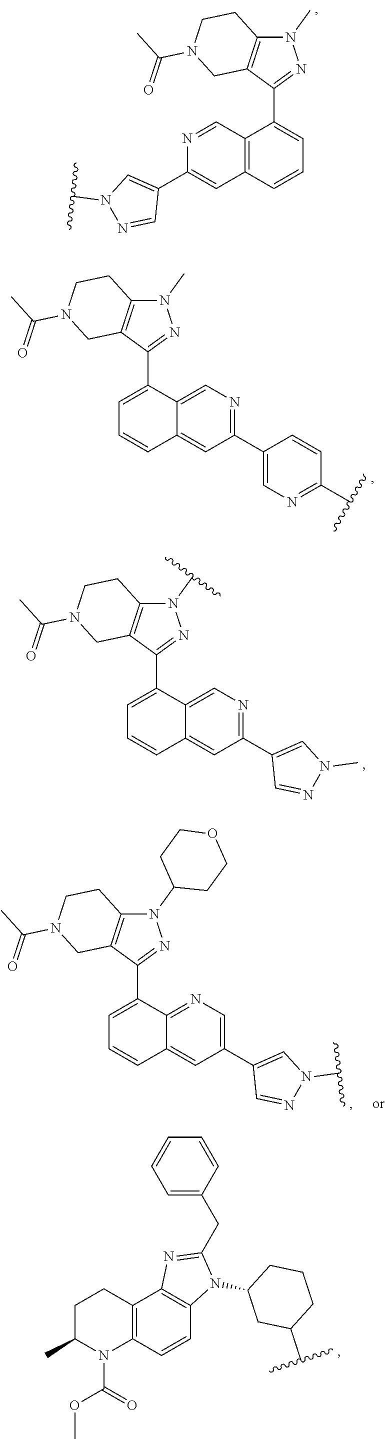

or a pharmaceutically acceptable salt or solvate thereof.

Embodiment 92. The compound of any one of embodiments 14-89, or a pharmaceutically acceptable salt or solvate thereof, wherein SB-CBP/p300 has one of the following structures:

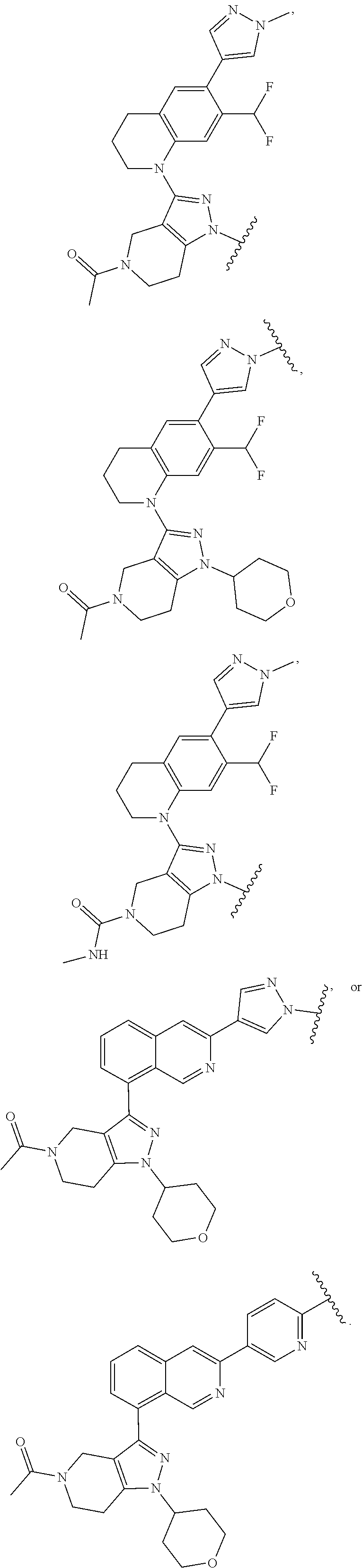

Embodiment 93. The compound of any one of embodiments 14-89, or a pharmaceutically acceptable salt or solvate thereof, wherein SB-CBP/p300 has one of the following structures:

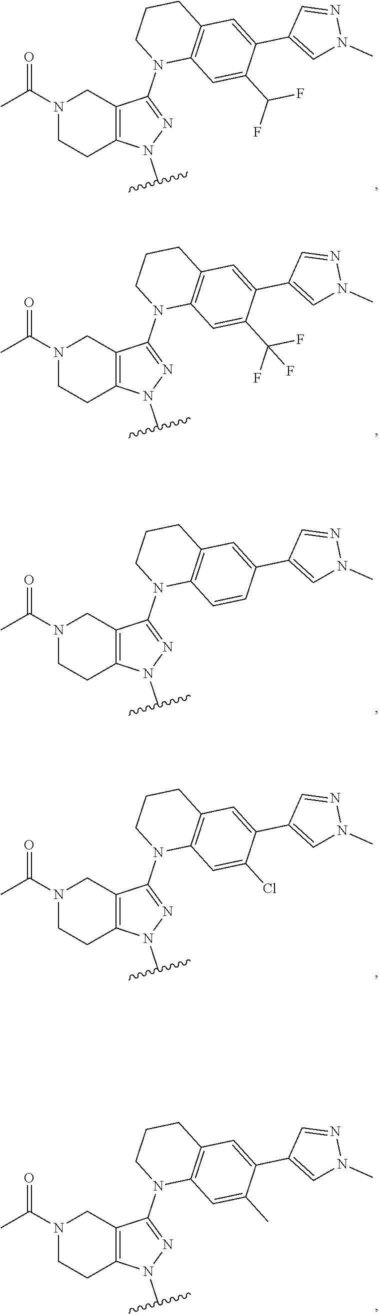

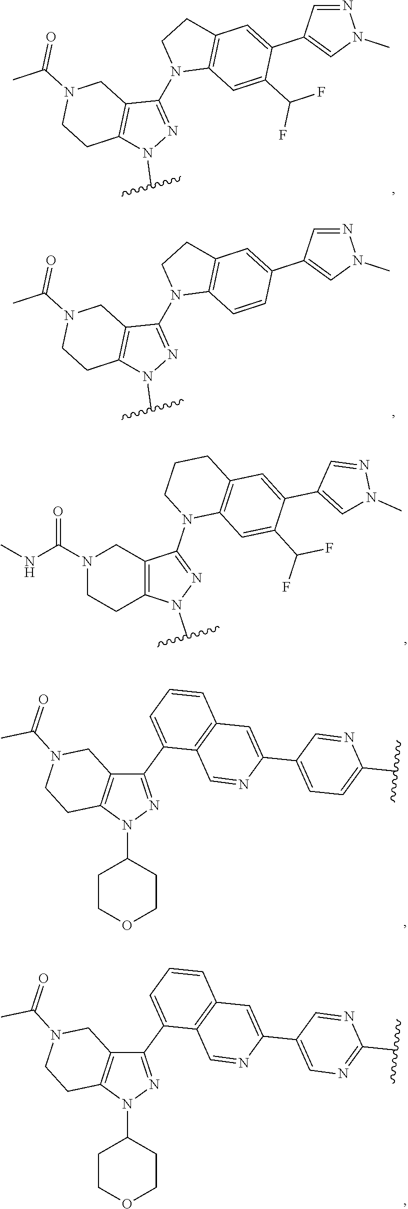

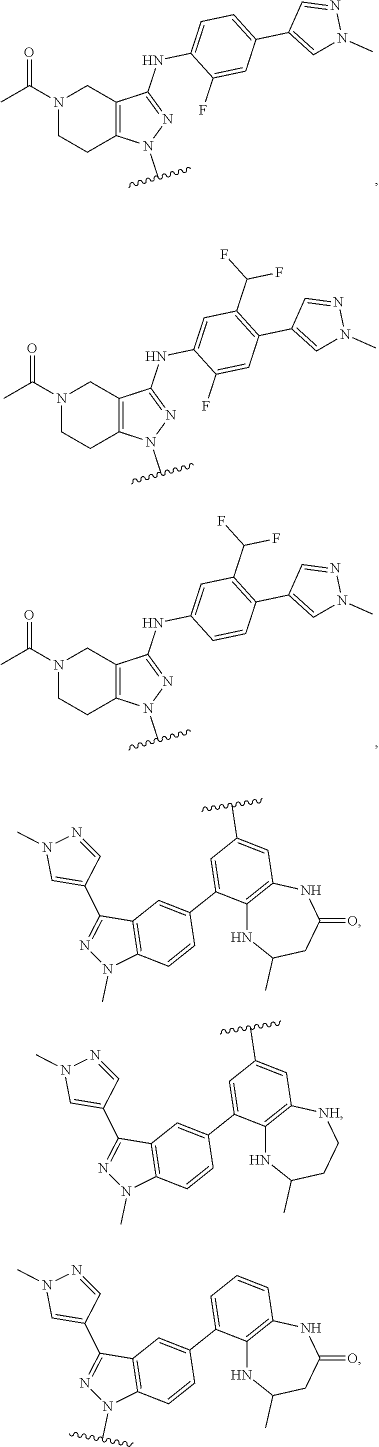

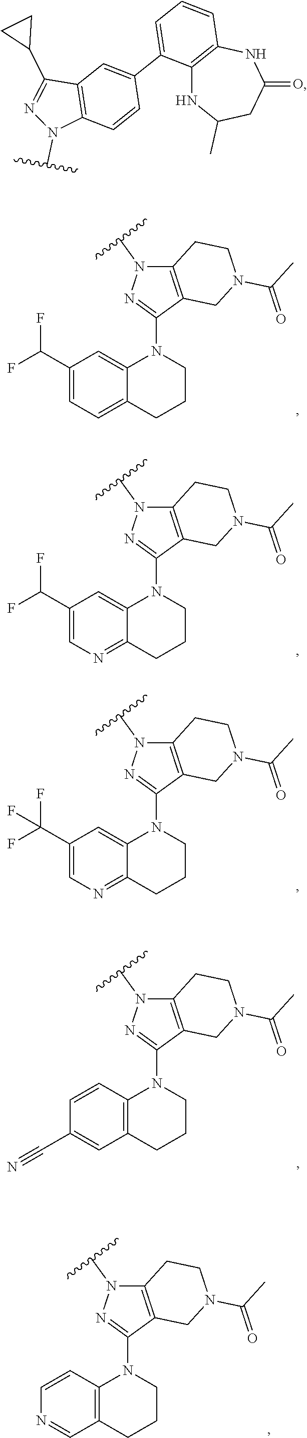 [SMILES: *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2 ; *n1nc(N2CCCc3cc(C#N)ccc32)c2c1CCN(C(C)=O)C2 ; *n1nc(N2CCCc3cnccc32)c2c1CCN(C(C)=O)C2]

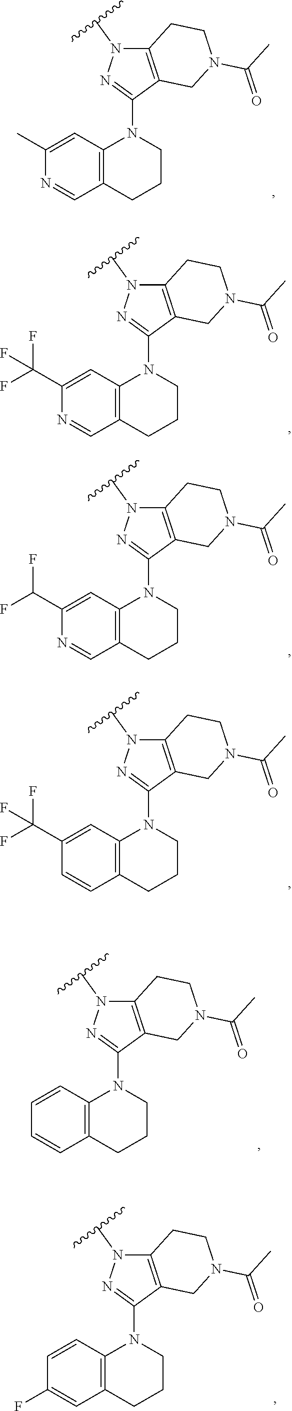 [SMILES: *n1nc(N2CCCc3cnc(C)cc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3ccc(C(F)(F)F)cc32)c2c1CCN(C(C)=O)C2 ; *n1nc(N2CCCc3ccccc32)c2c1CCN(C(C)=O)C2 ; *n1nc(N2CCCc3cc(F)ccc32)c2c1CCN(C(C)=O)C2]

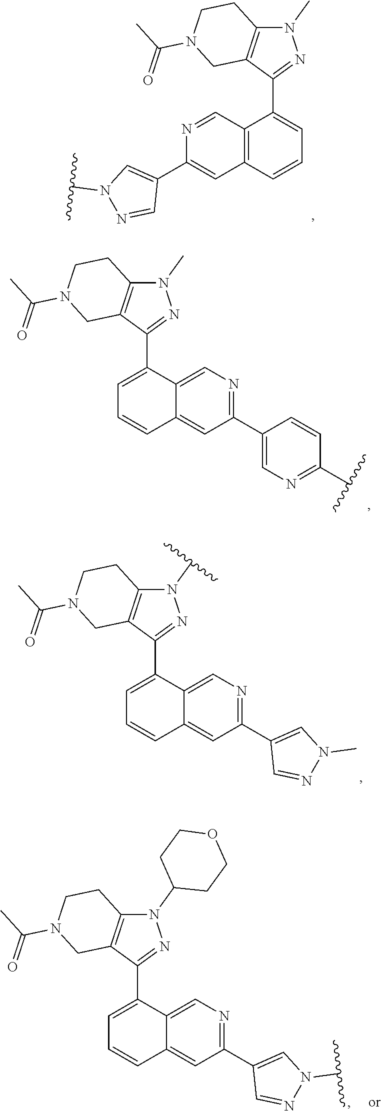

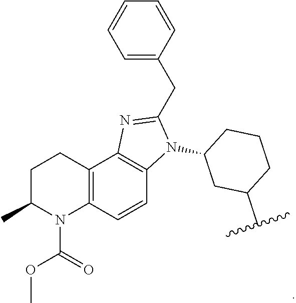 [SMILES: *C1CCC[C@@H](n2c(Cc3ccccc3)nc3c4c(ccc32)N(C(=O)OC)[C@@H](C)CC4)C1]

Embodiment 94. The compound of any one of embodiments 1-93, or a pharmaceutically acceptable salt or solvate thereof, wherein L is absent.

Embodiment 95. The compound of any one of embodiments 1-93, or a pharmaceutically acceptable salt or solvate thereof, wherein L comprises substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted 3- to 10-membered heterocycloalkyl, substituted or unsubstituted aryl, substituted or unsubstituted heteroaryl, or combinations thereof.

Embodiment 96. The compound of any one of embodiments 1-93, or a pharmaceutically acceptable salt or solvate thereof, wherein L is absent or

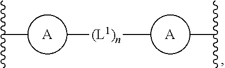

wherein:
each A is independently absent, substituted or unsubstituted monocyclic C₃-C₁₀cycloalkyl, substituted or unsubstituted bridged bicyclic C₃-C₁₀cycloalkyl, substituted or unsubstituted fused bicyclic C₃-C₁₀cycloalkyl, substituted or unsubstituted spiro bicyclic C₃-C₁₀cycloalkyl, substituted or unsubstituted monocyclic 3- to 10-membered heterocycloalkyl, substituted or unsubstituted bridged bicyclic 3- to 10-membered heterocycloalkyl, substituted or unsubstituted fused bicyclic 3- to 10-membered heterocycloalkyl, substituted or unsubstituted spiro bicyclic 3- to 10-membered heterocycloalkyl, substituted or unsubstituted aryl, or substituted or unsubstituted heteroaryl, wherein each A is independently unsubstituted or substituted with x R^{2b};
each L¹ is independently absent,

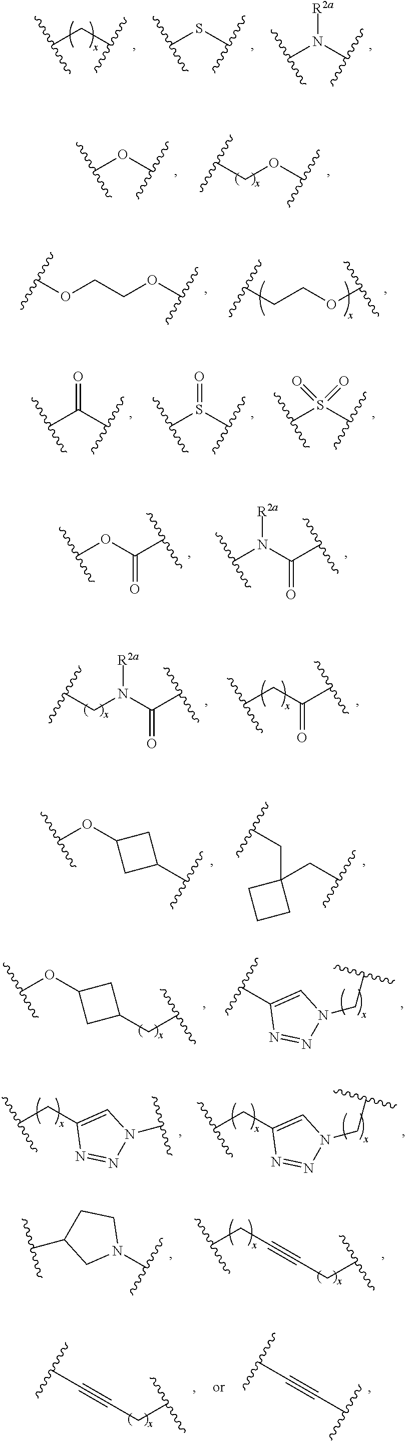

wherein each L¹ is independently unsubstituted or substituted with x R^{2b};
   n is 1, 2, 3, 4, 5, or 6;
each x is independently 1, 2, 3, 4, 5, 6, 7, or 8;
each R^{2a} is independently hydrogen or substituted or unsubstituted C₁-C₆alkyl; and
each R^{2b} is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, —OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b});
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 97. The compound of embodiment 96, or a pharmaceutically acceptable salt or solvate thereof, wherein:
each A is independently absent,

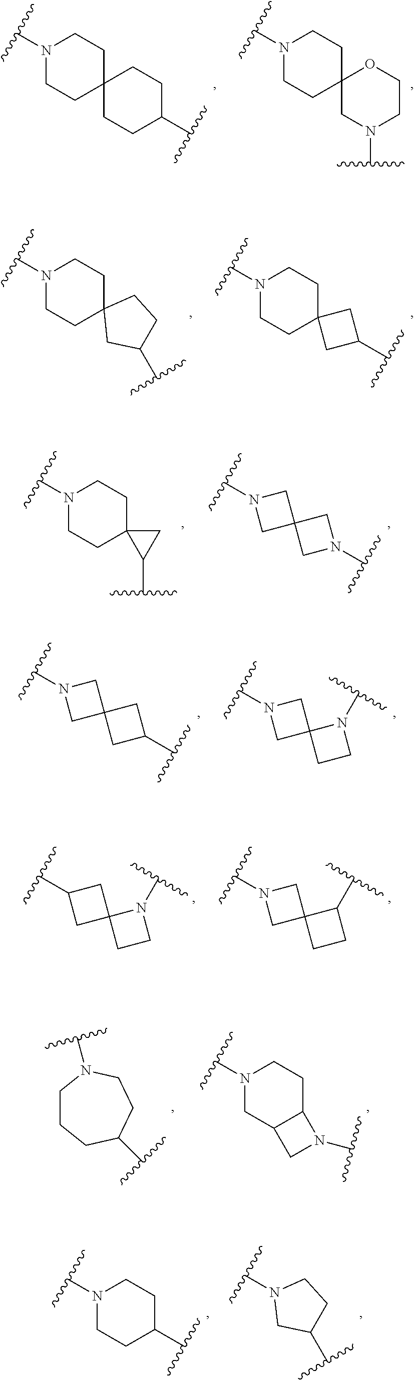

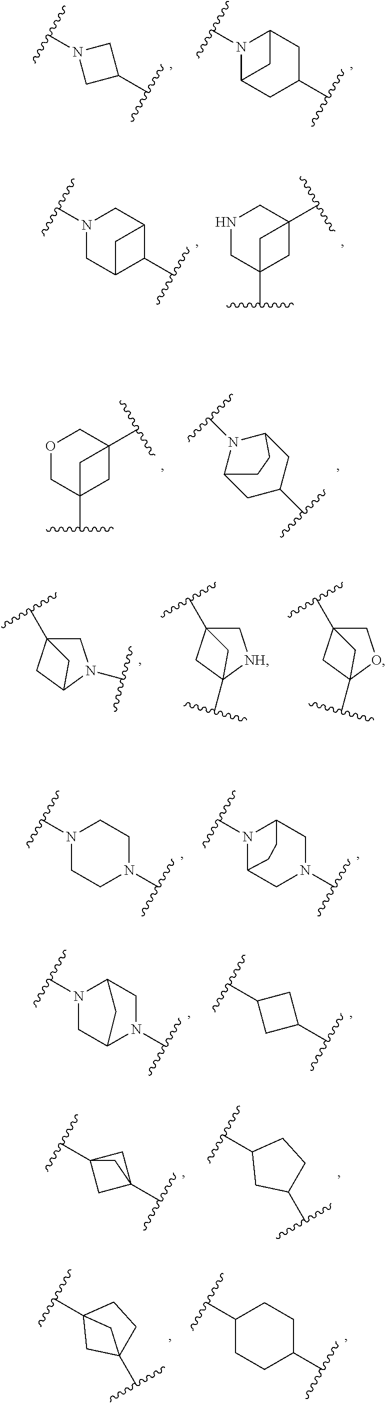

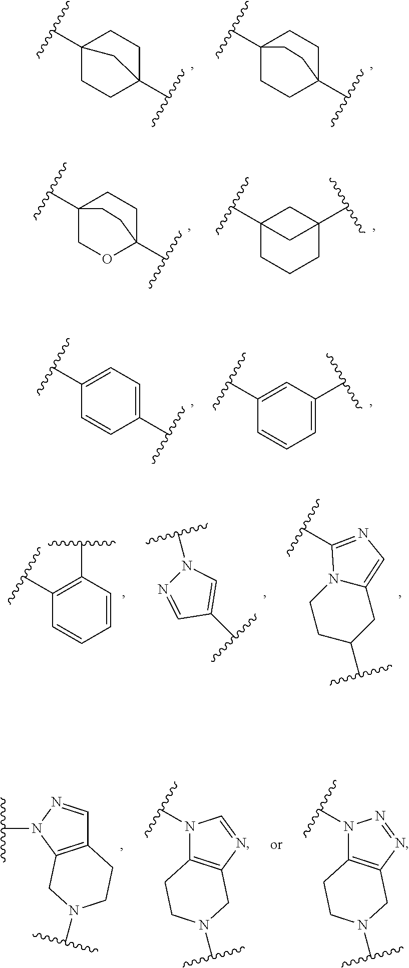

wherein each A is independently unsubstituted or substituted with x R^{2b}; (L¹)ₙ is absent

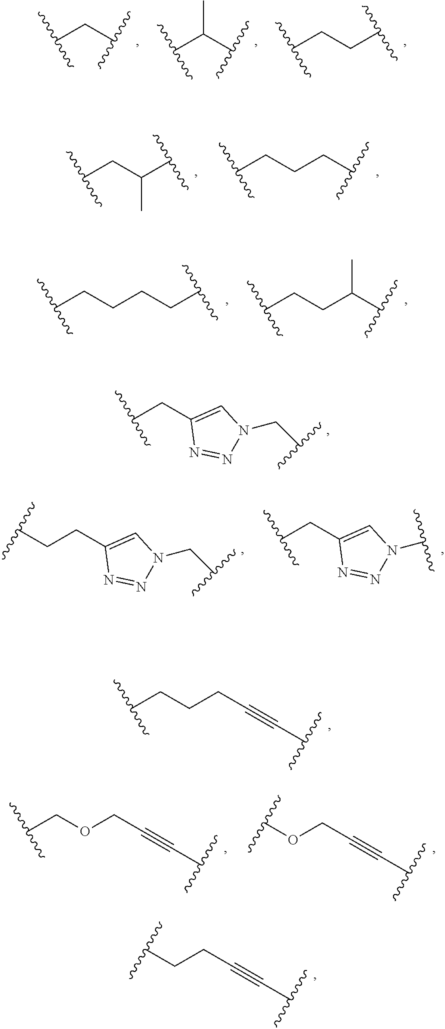

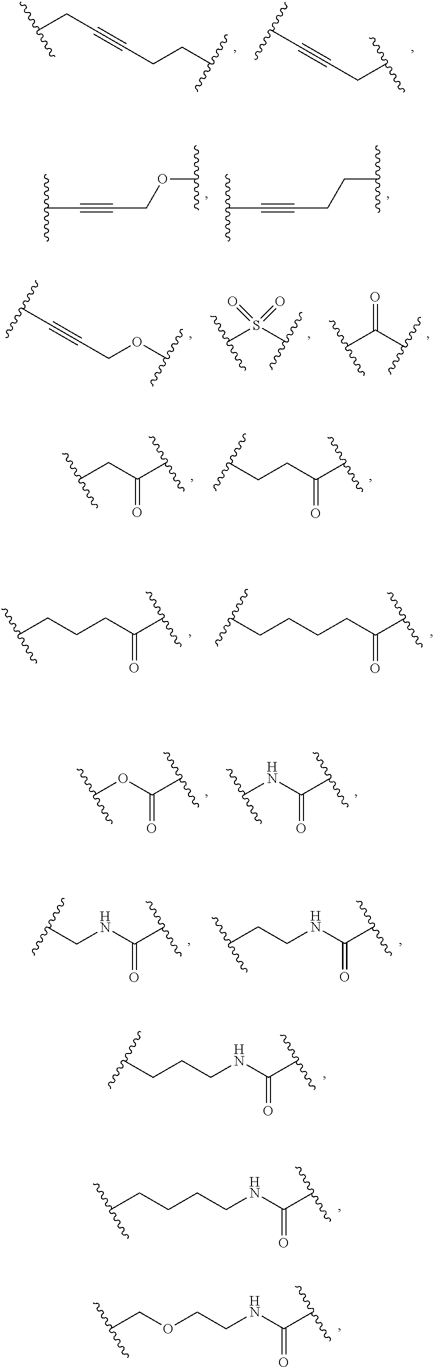

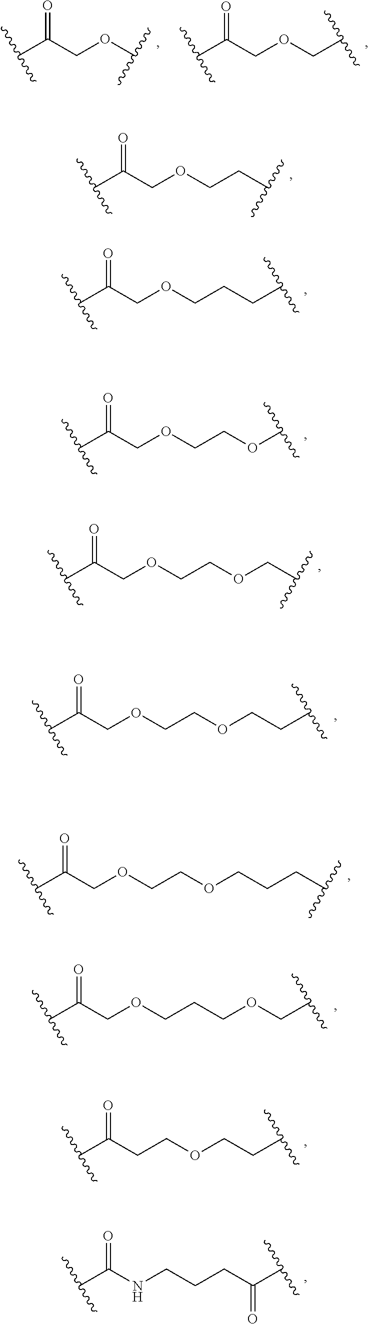

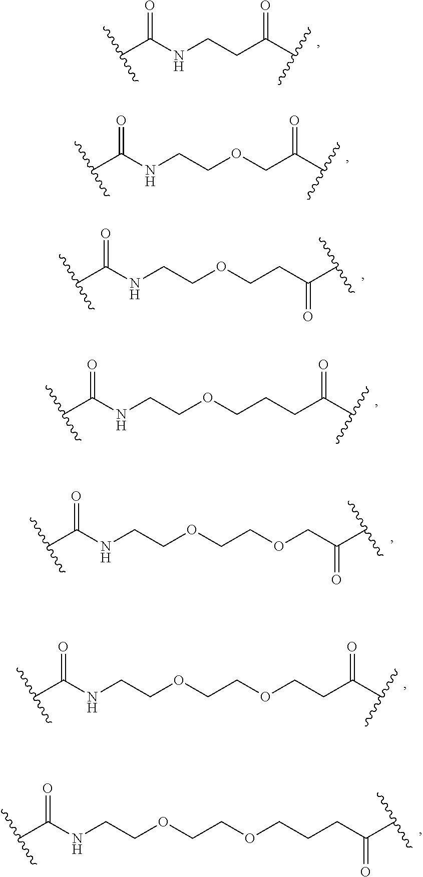

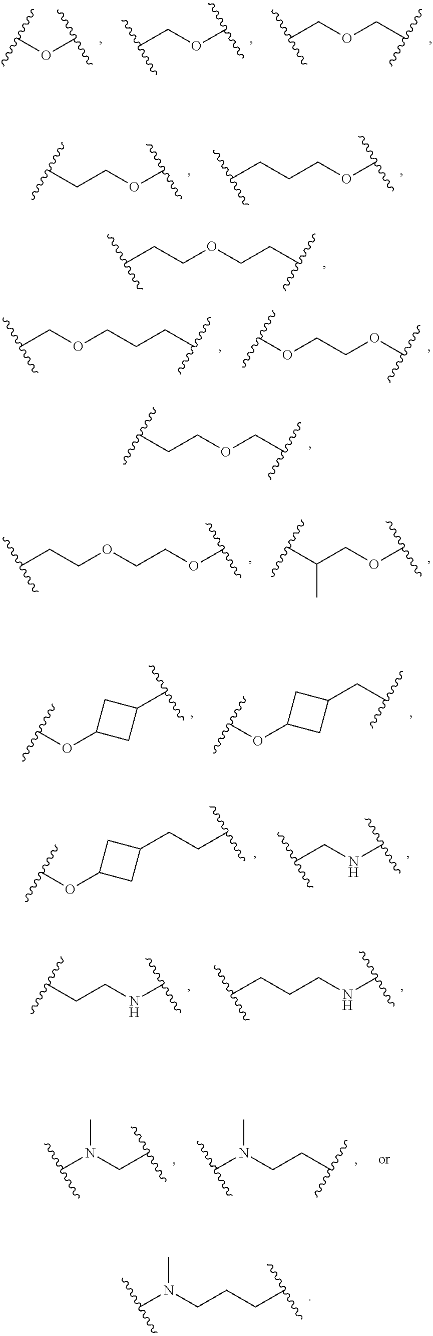

Embodiment 98. The compound of any one of embodiments 1-93, or a pharmaceutically acceptable salt or solvate thereof, wherein L is absent or wherein:

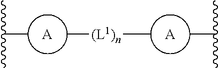

each A is independently absent,

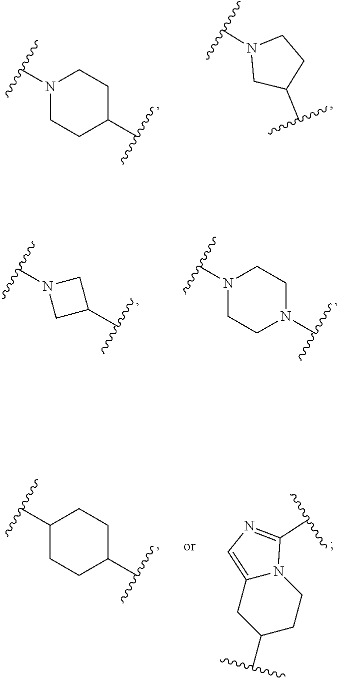 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]

each L is independently absent,

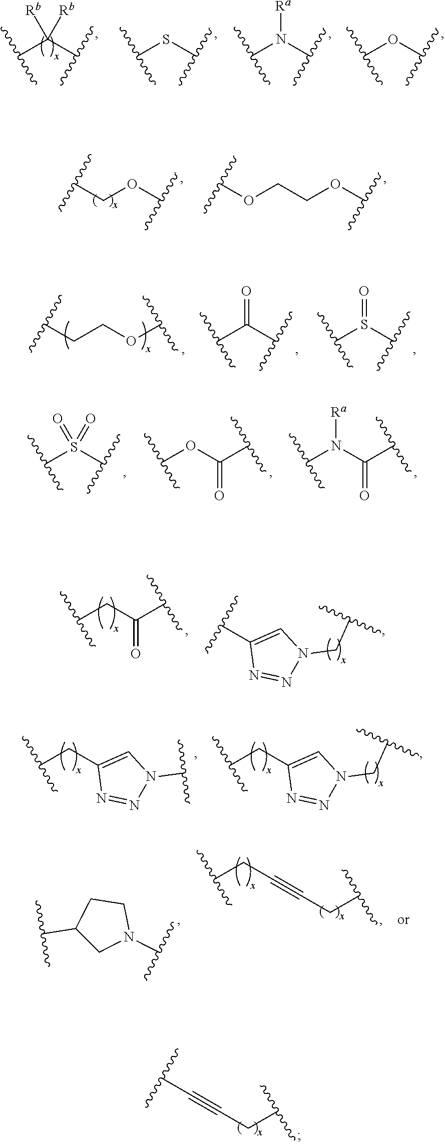

n is 1, 2, or 3; each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

Embodiment 99. The compound of any one of embodiments 96-98, or a pharmaceutically acceptable salt or solvate thereof, wherein:
(L¹)ₙ is absent,

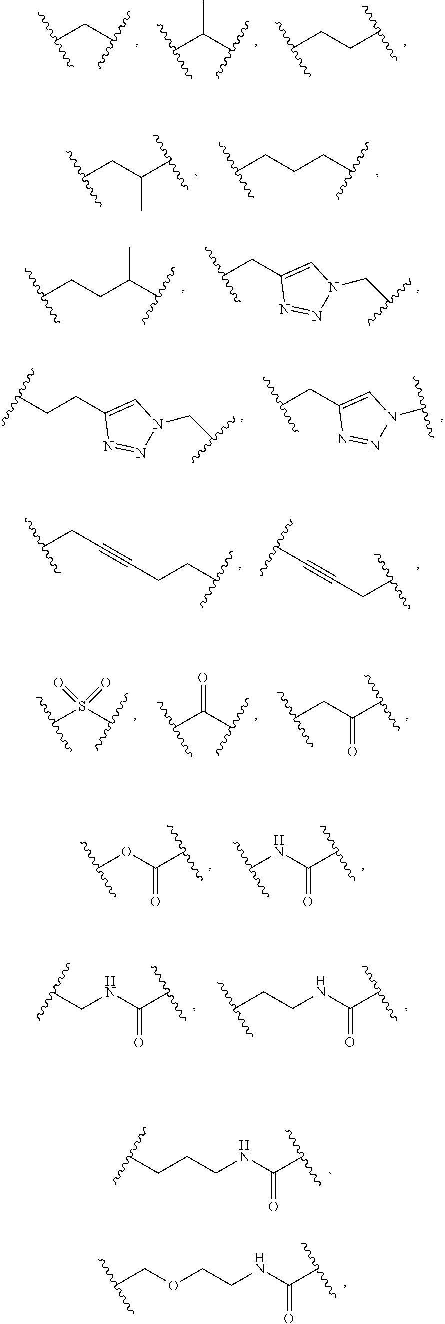

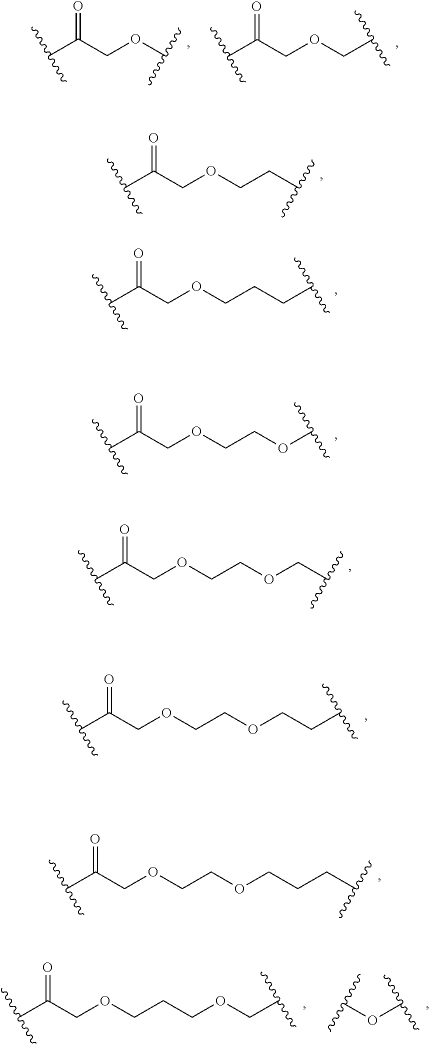

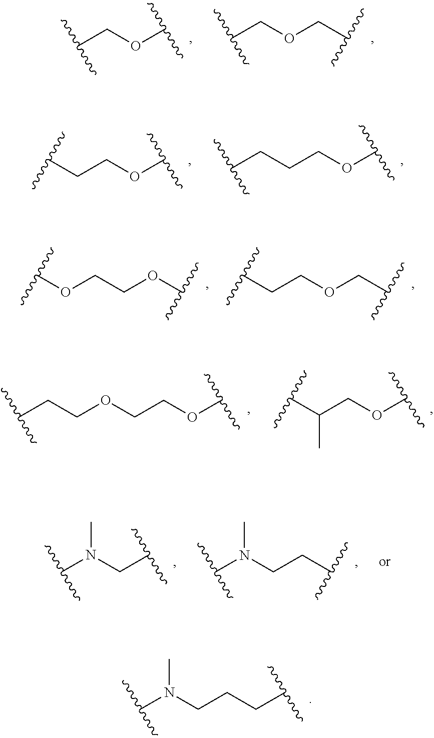

Embodiment 100. The compound of any one of embodiments 1-99, or a pharmaceutically acceptable salt or solvate thereof, wherein L has one of the following structures:

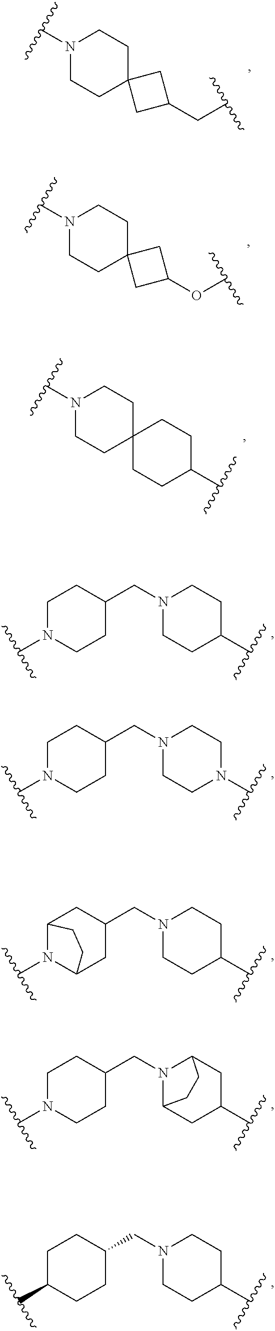

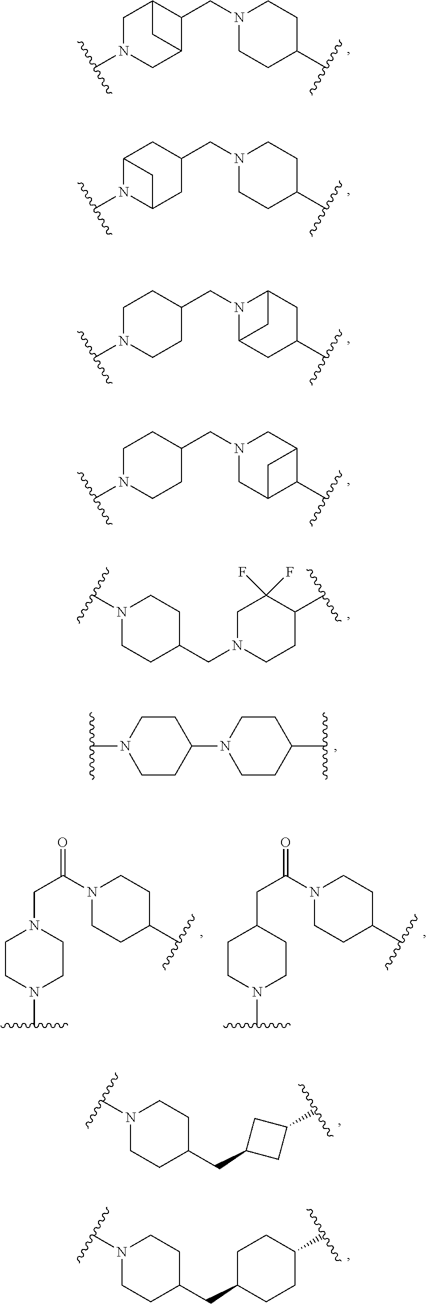

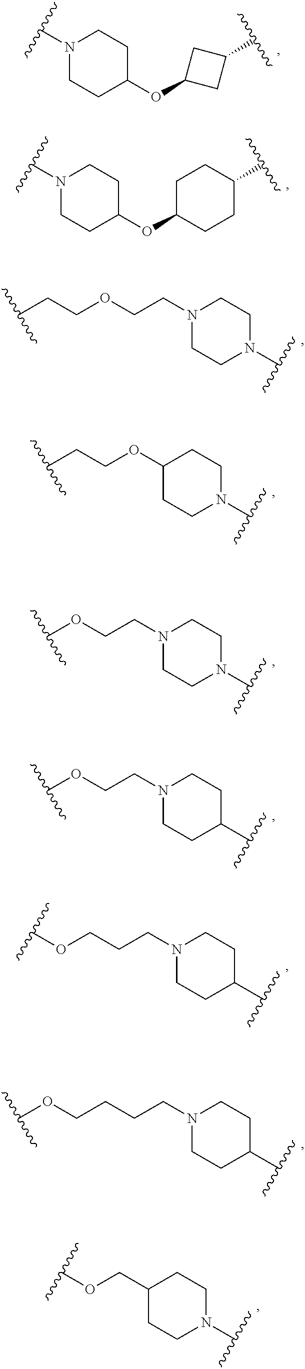

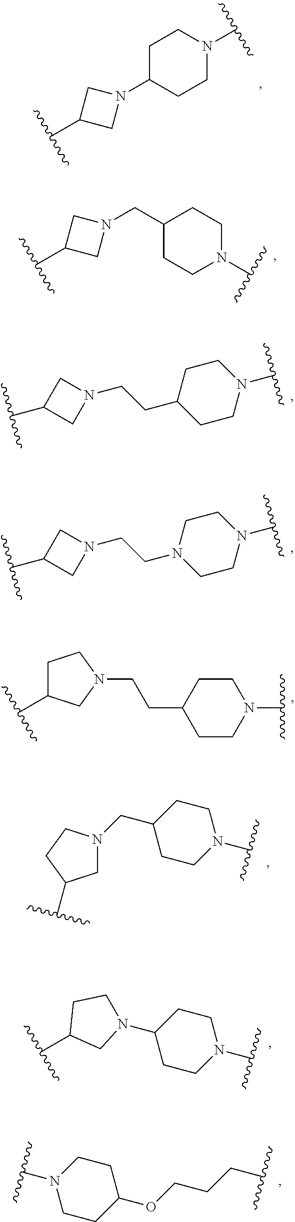

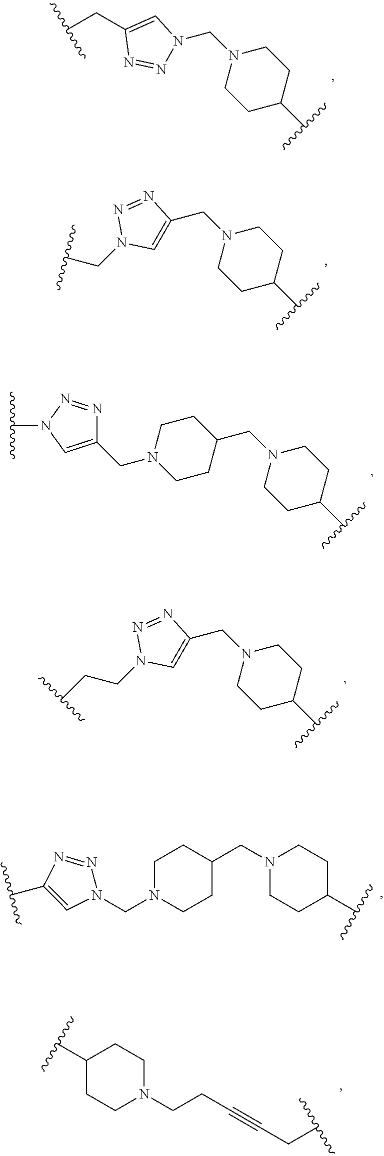 [SMILES: *Cc1cn(CN2CCC(*)CC2)nn1 ; *Cn1cc(CN2CCC(*)CC2)nn1 ; *C1CCN(CC2CCN(Cc3cn(*)nn3)CC2)CC1 ; *CCn1cc(CN2CCC(*)CC2)nn1 ; *c1cn(CN2CCC(CN3CCC(*)CC3)CC2)nn1 ; *CC#CCCN1CCC(*)CC1]

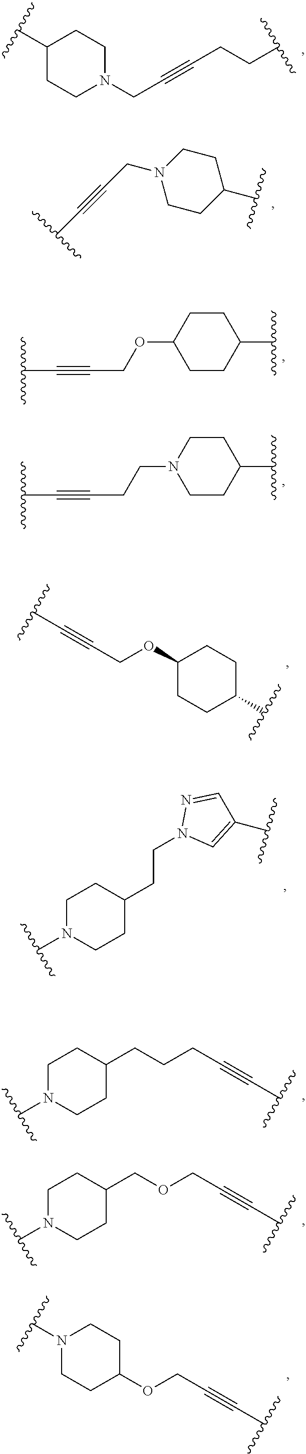

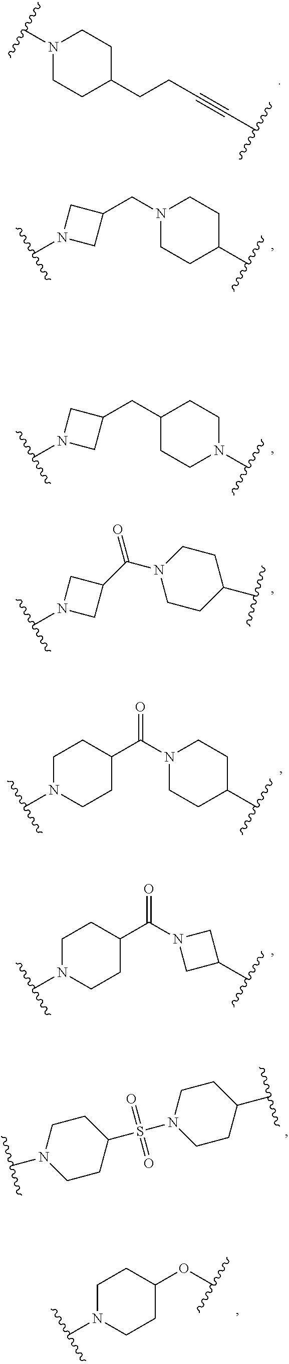

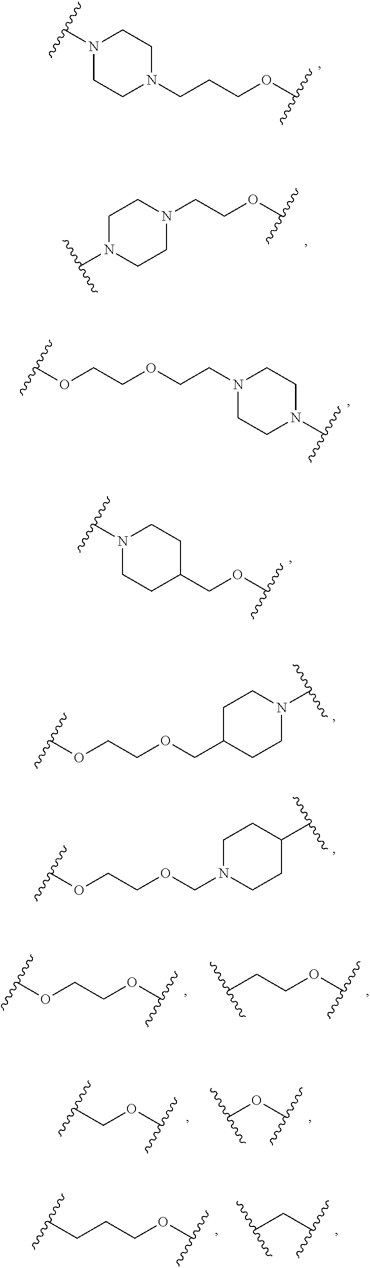

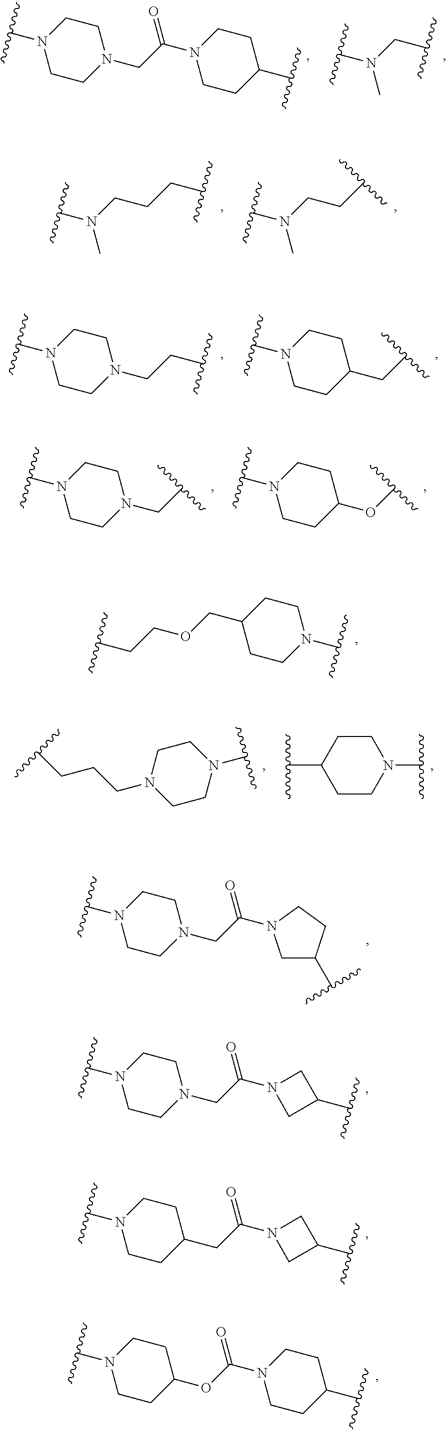

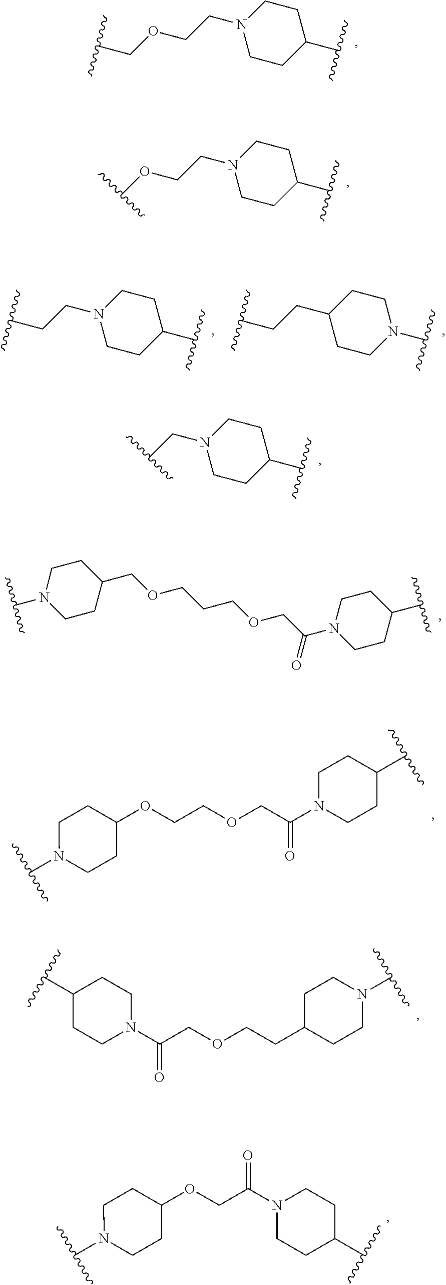

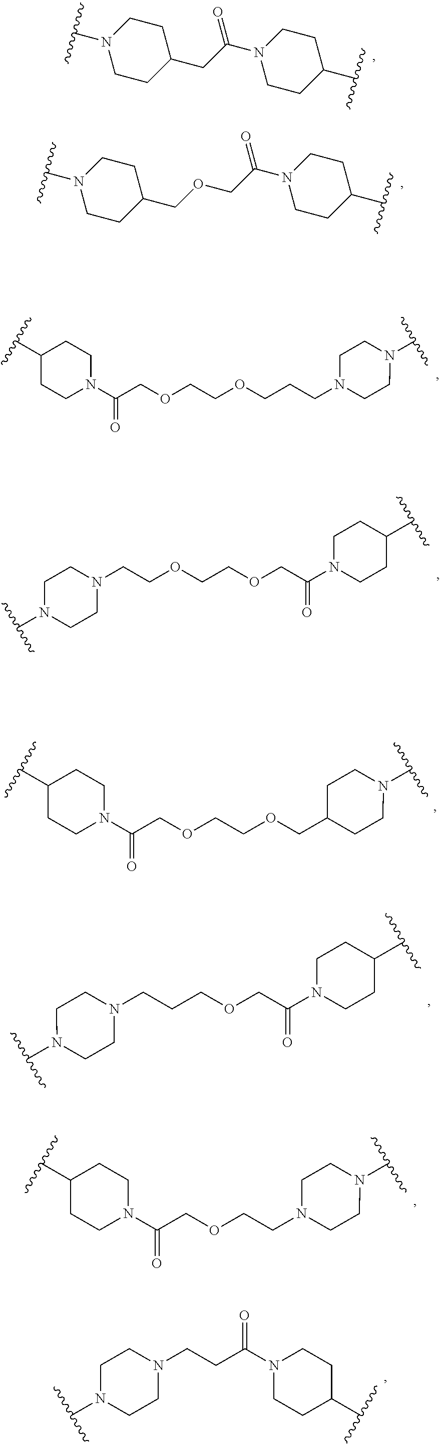

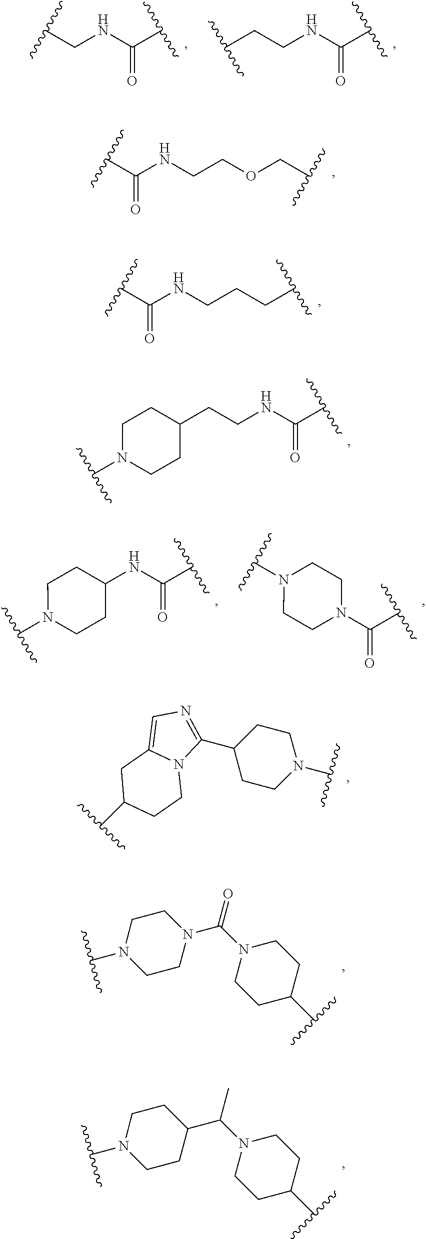

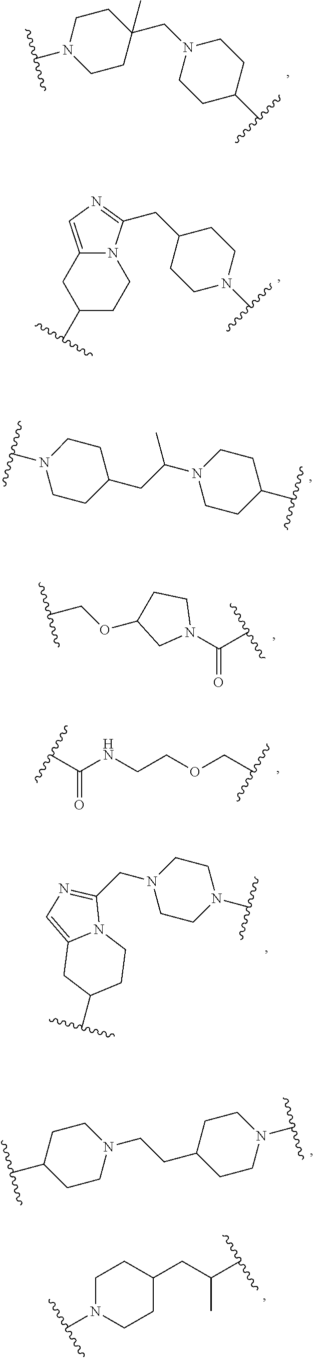

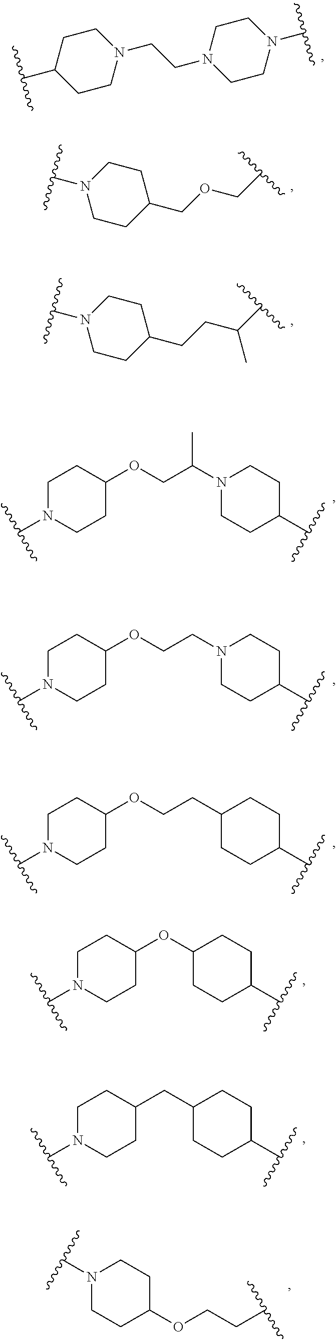

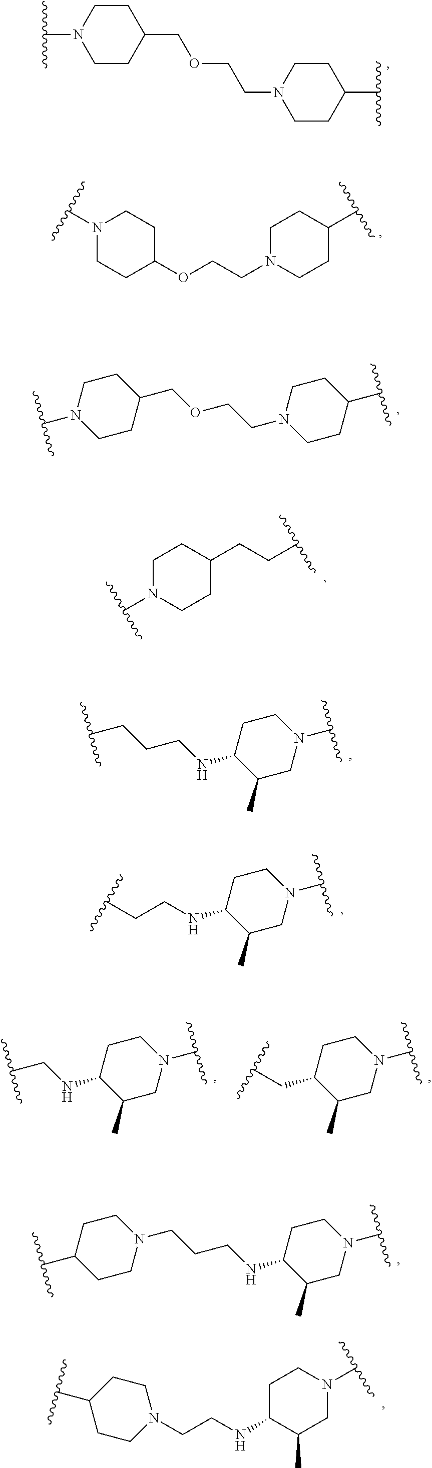

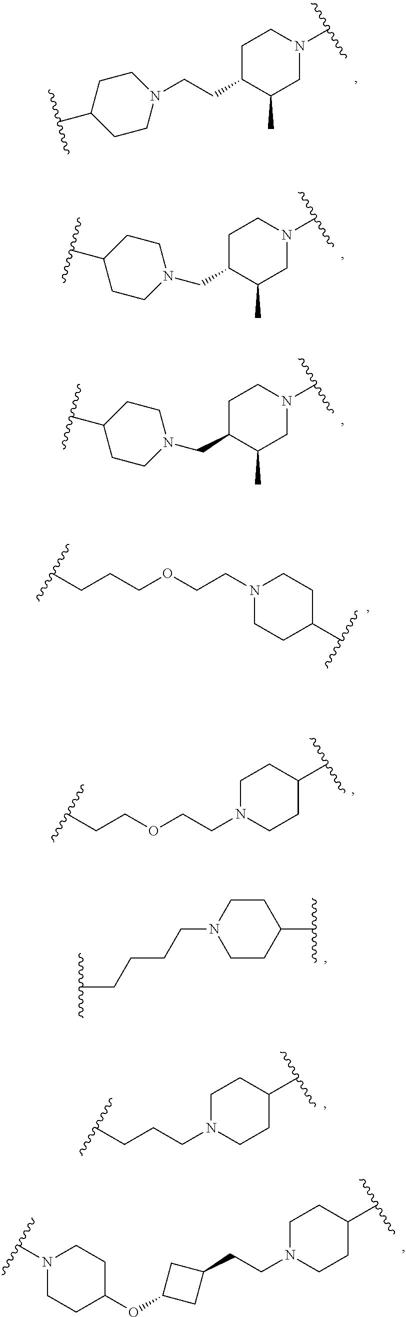

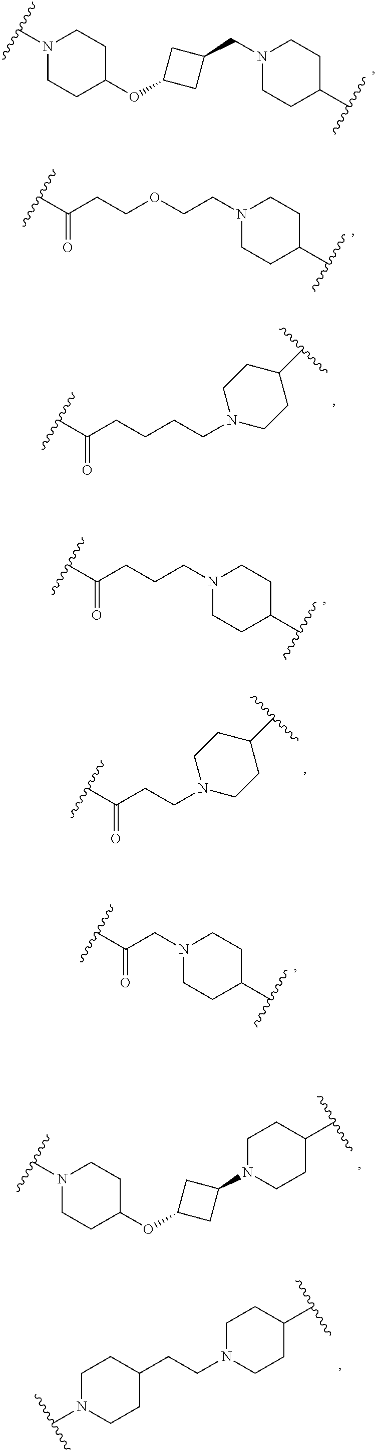

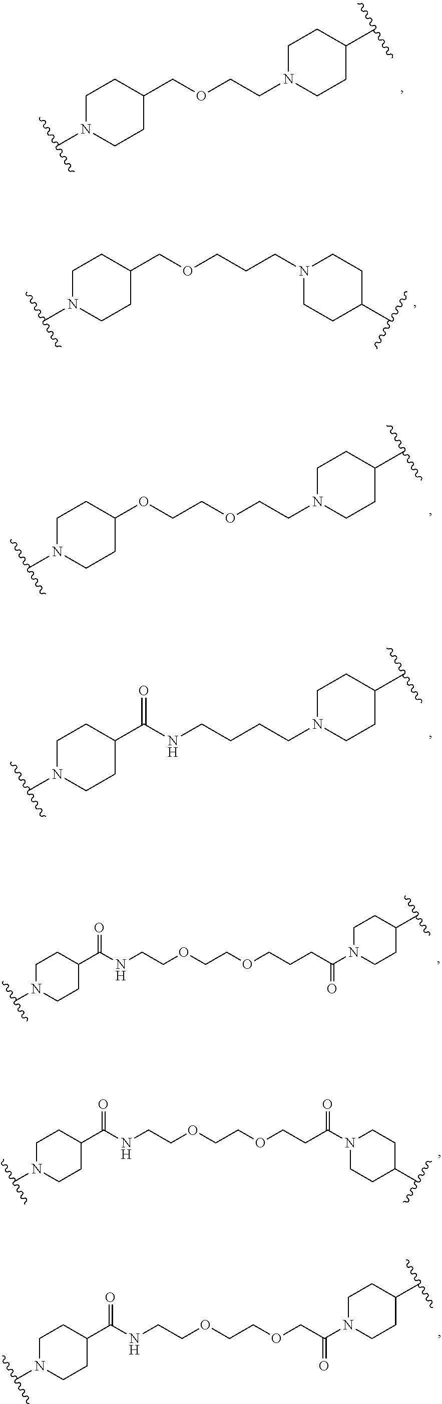

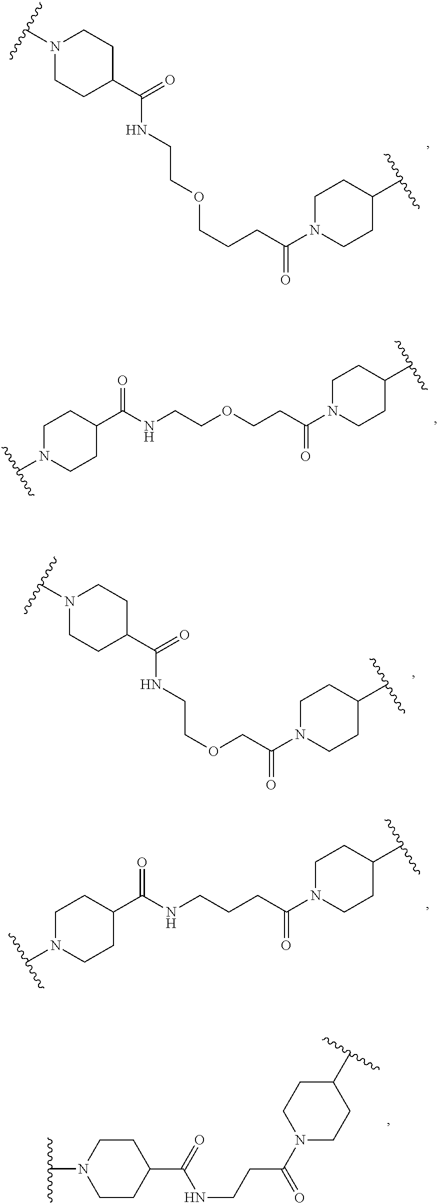 [SMILES: *C1CCN(C(=O)CCCOCCNC(=O)C2CCN(*)CC2)CC1 ; *C1CCN(C(=O)CCOCCNC(=O)C2CCN(*)CC2)CC1 ; *C1CCN(C(=O)COCCNC(=O)C2CCN(*)CC2)CC1 ; *C1CCN(C(=O)CCCNC(=O)C2CCN(*)CC2)CC1 ; *C1CCN(C(=O)CCNC(=O)C2CCN(*)CC2)CC1]

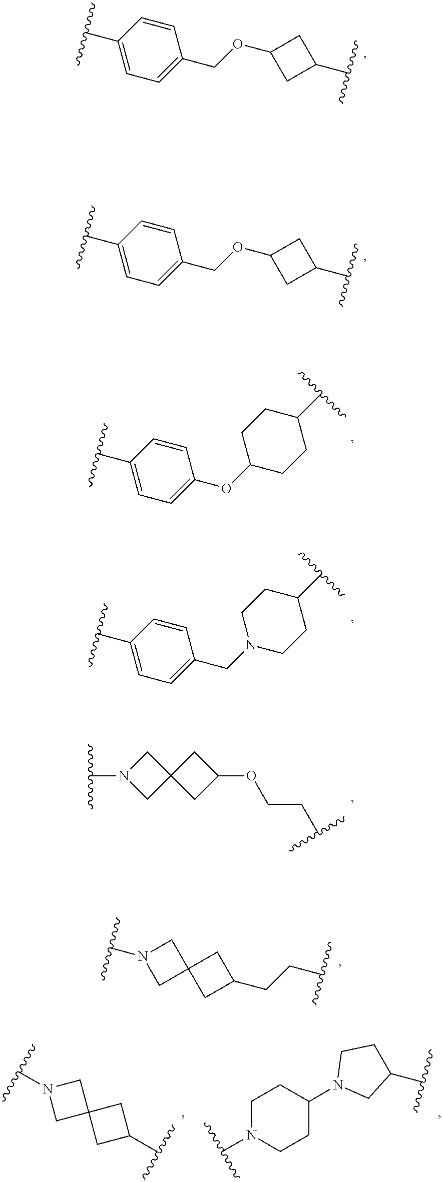

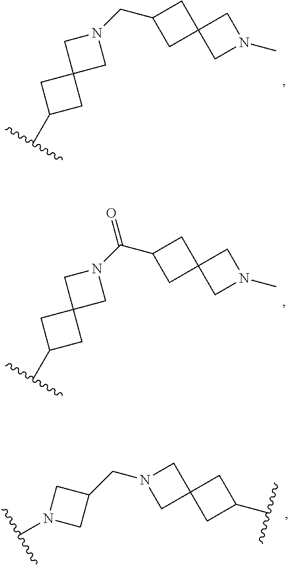 [SMILES: *C1CC2(C1)CN(CC1CC3(C1)CN(C)C3)C2 ; *C1CC2(C1)CN(C(=O)C1CC3(C1)CN(C)C3)C2 ; *C1CC2(C1)CN(CC1CN(*)C1)C2]

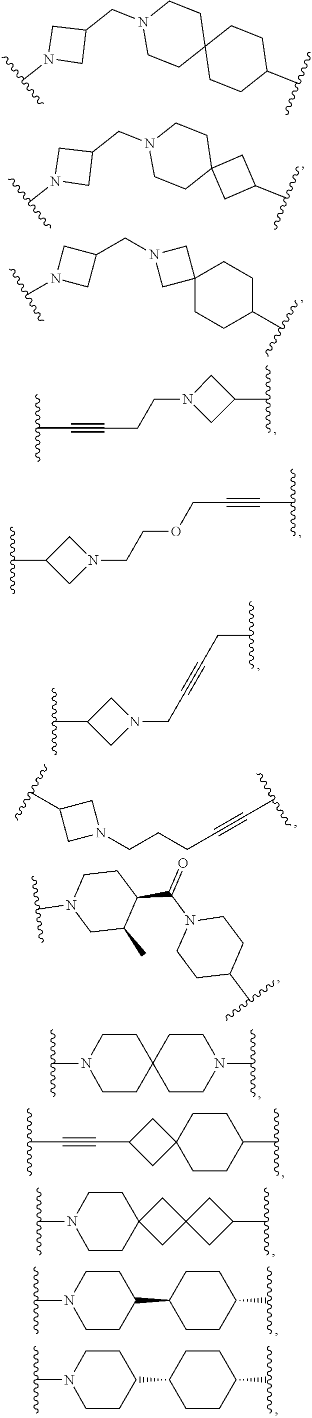

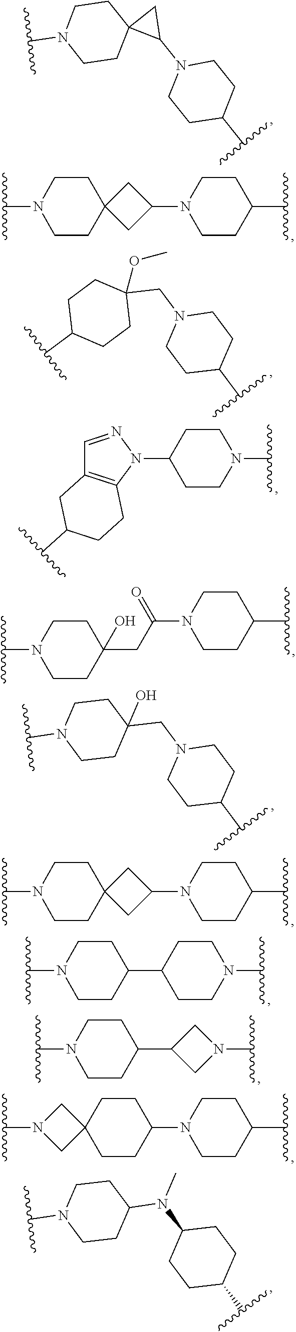

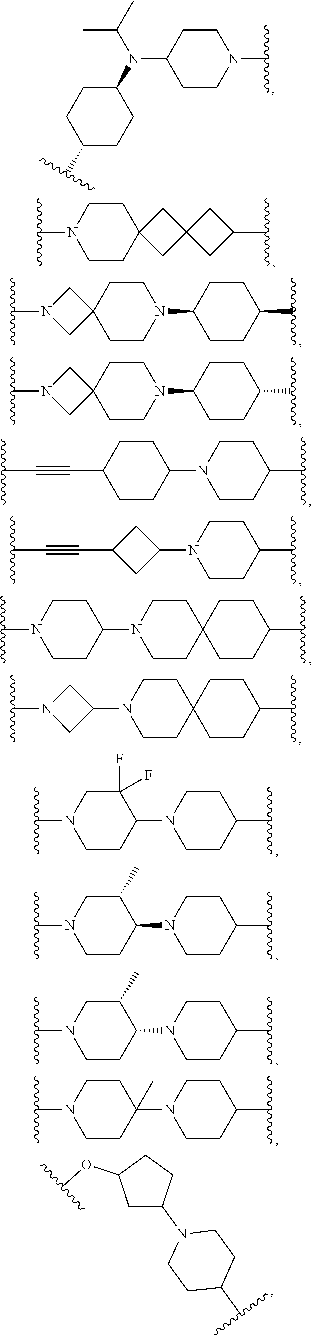

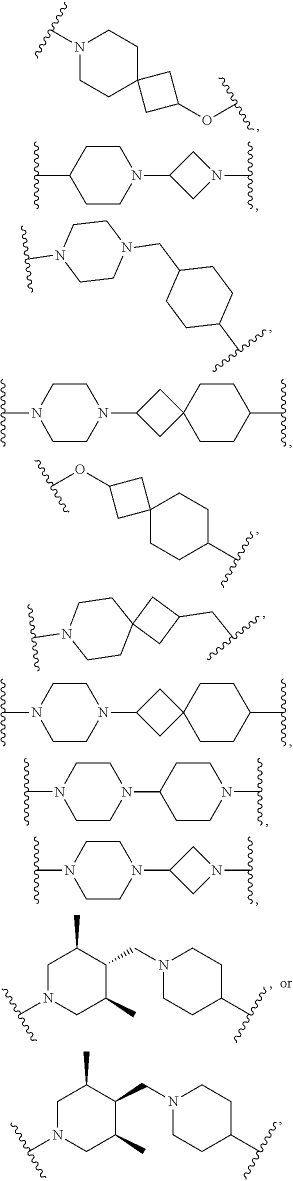

or a pharmaceutically acceptable salt or solvate thereof.

Embodiment 101. The compound of any one of embodiments 1-99, or a pharmaceutically acceptable salt or solvate thereof, wherein L has one of the following structures:

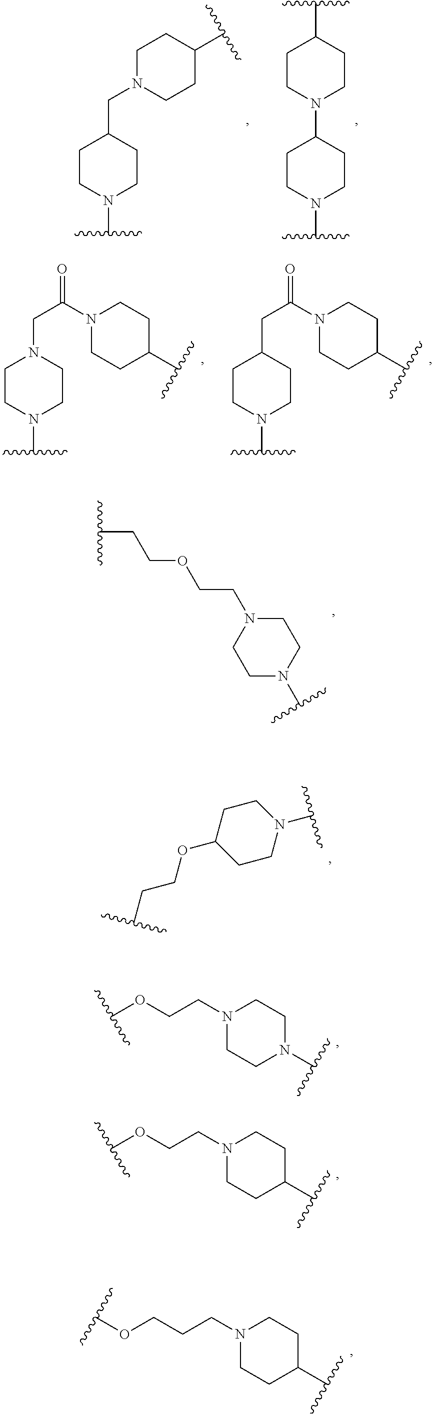

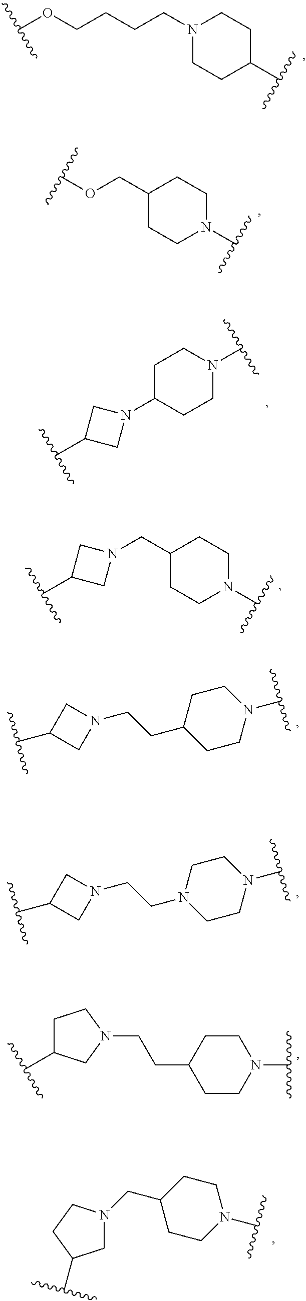

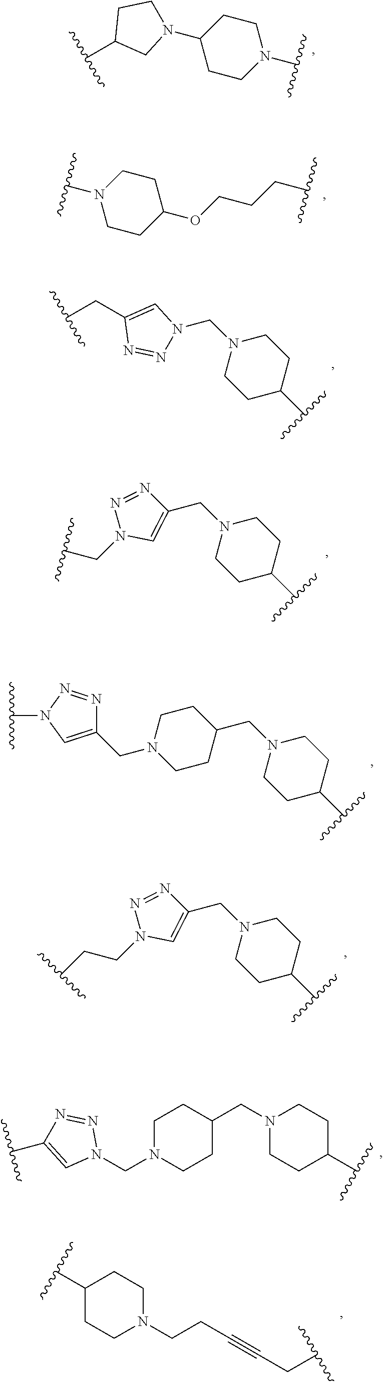

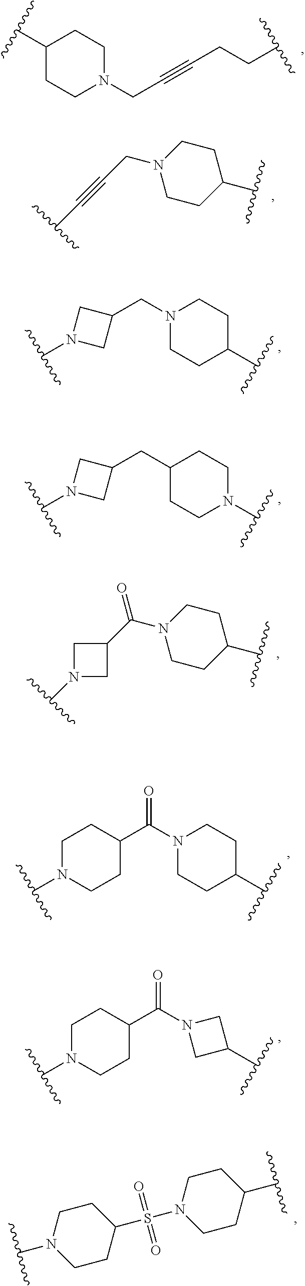

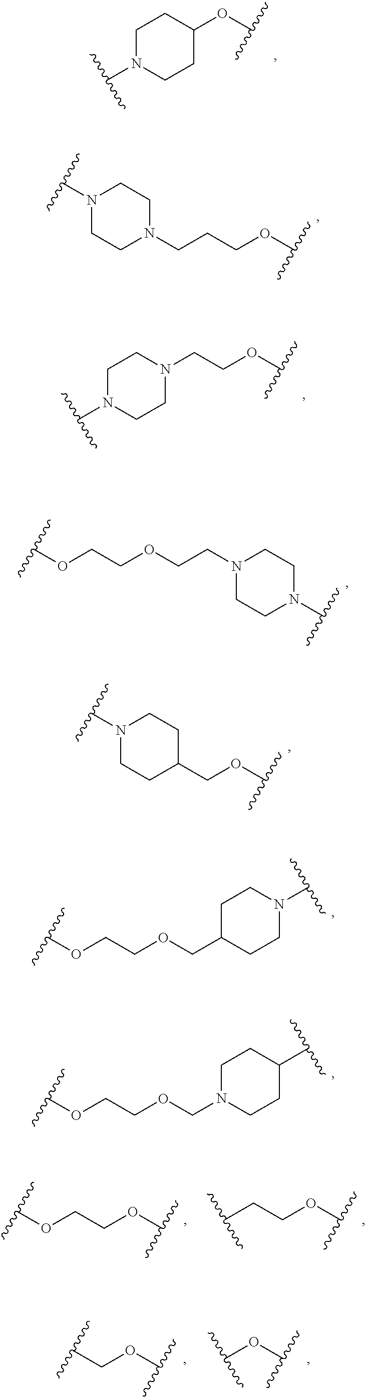

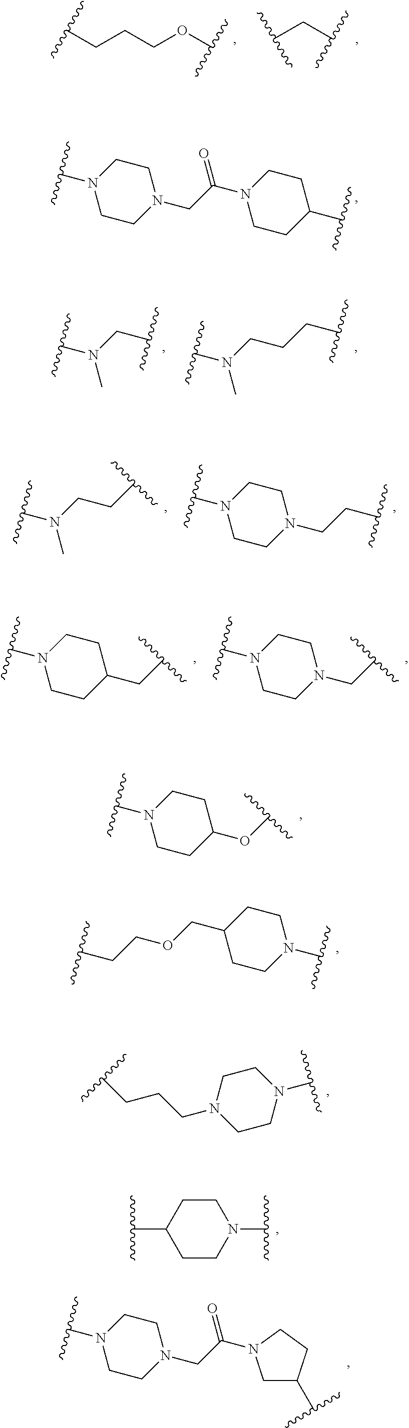

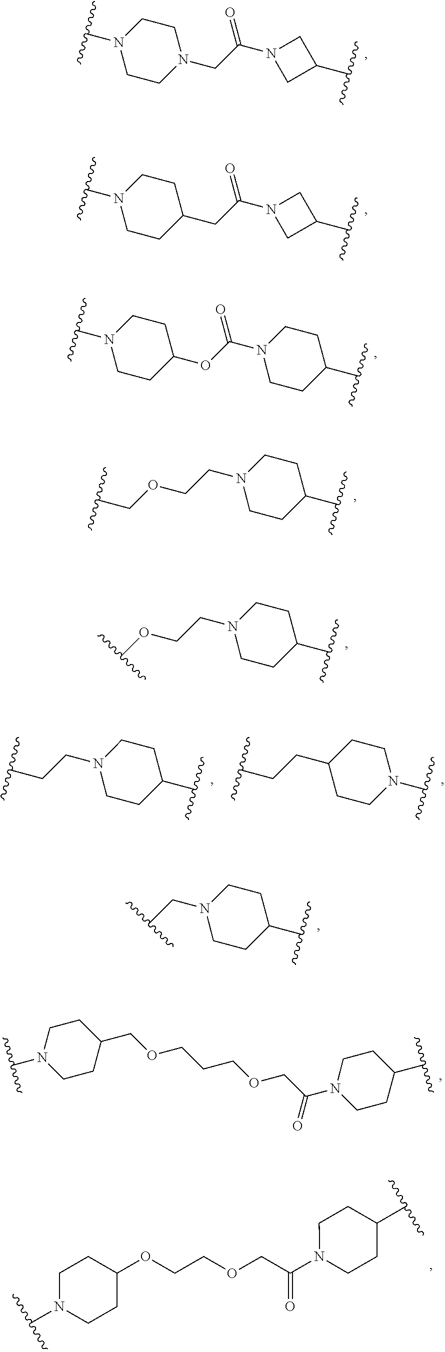

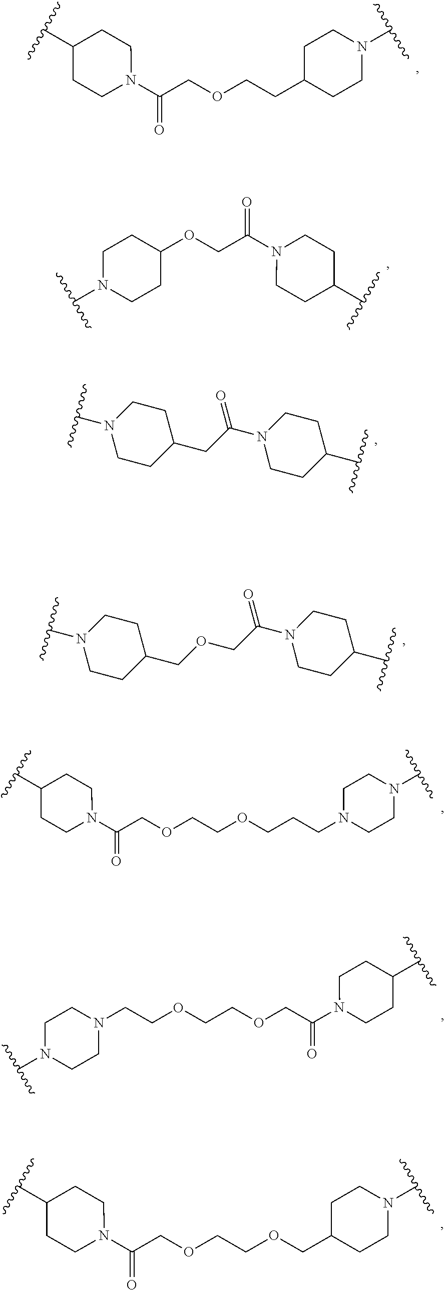

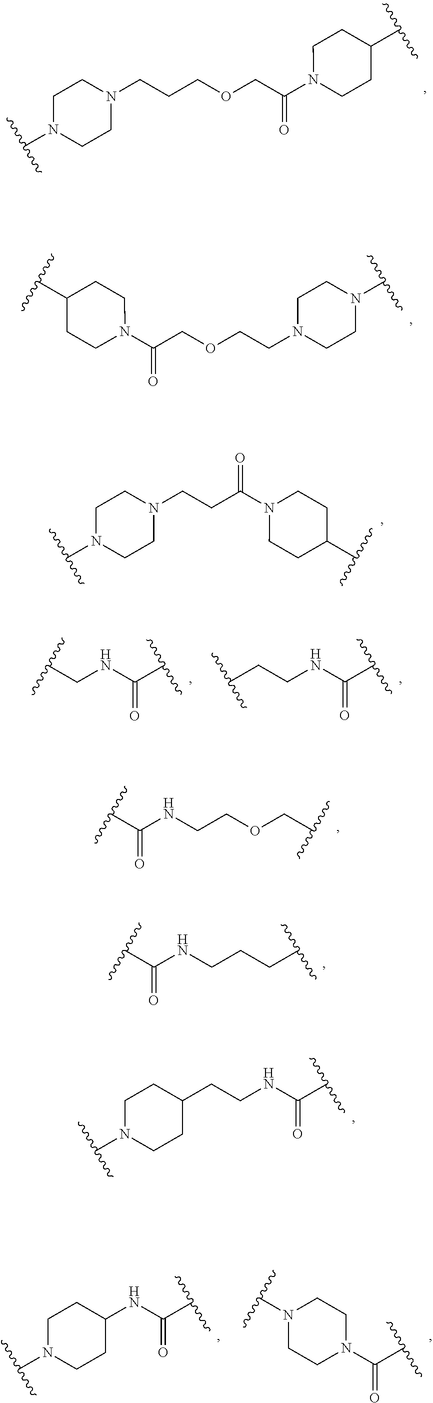

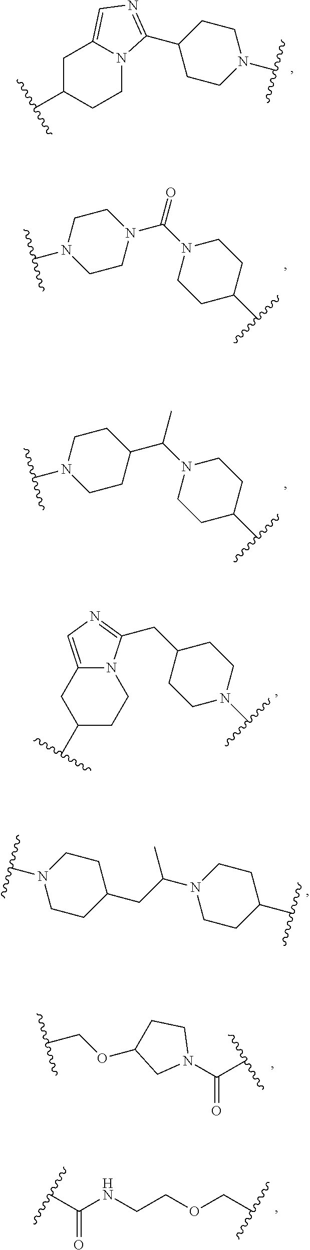

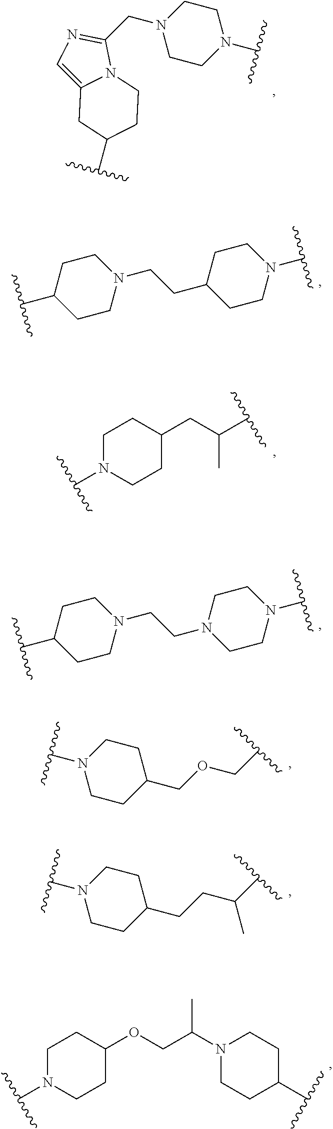

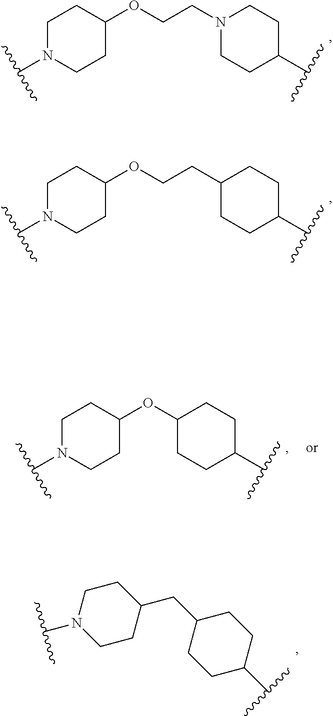 [SMILES: *C1CCN(CCOC2CCN(*)CC2)CC1 ; *C1CCC(CCOC2CCN(*)CC2)CC1 ; *C1CCC(OC2CCN(*)CC2)CC1 ; *C1CCC(CC2CCN(*)CC2)CC1]

or a pharmaceutically acceptable salt or solvate thereof.

Embodiment 102. A compound of Table 1, or a pharmaceutically acceptable salt or solvate thereof.

Embodiment 103. A stable ternary complex comprising:
a. one or more disease-dependent proteins (DDPs);
b. Androgen Receptor (AR); and
c. heterobifunctional conditional inhibitor compound of any one of embodiments 1-105;
wherein DDP and AR are present in a cell of interest (COI) and the relative abundance of the AR in the COI is greater than the relative abundance of the DDP in the COI.

Embodiment 104. The stable ternary complex of embodiment 103, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

Embodiment 105. The stable ternary complex of embodiment 103, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

Embodiment 106. The stable ternary complex of embodiment 103, wherein the DDP is CREB-binding protein (CBP)/p300.

Embodiment 107. A stable ternary complex comprising:
a. CBP/p300;
b. Androgen receptor (AR); and
c. heterobifunctional conditional inhibitor compound of any one of embodiments 1-105;
wherein CBP/p300 and DP are present in a cell of interest (COI) and the relative abundance of the DP in the COI is greater than the relative abundance of CBP/p300 in the COI.

Embodiment 108. A method of selectively inhibiting the activity of a disease-dependent protein (DDP) in a cell of interest (COI) of a mammal comprising administering a heterobifunctional compound of any one of embodiments 1-102, or a pharmaceutically acceptable salt or solvate thereof, wherein the COI expresses the androgen receptor (AR).

Embodiment 109. The method of embodiment 108, wherein the heterobifunctional compound of any one of embodiments 1-102, or a pharmaceutically acceptable salt or solvate thereof, inhibits the activity of the DDP in the COI but does not inhibit the activity of the DDP in cells expressing the DDP and not expressing the AR.

Embodiment 110. The method of embodiment 108, wherein the AR is overexpressed, overactive or both overexpressed and overactive in the COI.

Embodiment 111. The method of any one of embodiments 108-110, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

Embodiment 112. The method of any one of embodiments 108-111, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

Embodiment 113. The method of any one of embodiments 108-111, wherein the DDP is CREB-binding protein (CBP)/p300.

Embodiment 114. A method of treating cancer in a mammal comprising administering to the mammal a heterobifunctional compound of any one of embodiments 1-102.

Embodiment 115. A method of treating cancer in a mammal comprising administering to the mammal a therapeutically effective amount of a compound of any one of embodiments 1-102, or a pharmaceutically acceptable salt or solvate thereof.

Embodiment 116. The method of embodiment 115, wherein the cancer is a hormone dependent cancer.

Embodiment 117. The method of embodiment 115, wherein the cancer is prostate cancer.

Embodiment 118. A method of treating an androgen receptor dependent or androgen receptor mediated disease or condition in mammal comprising administering to the mammal a therapeutically effective amount of a compound according to any one of embodiments 1-102, or a pharmaceutically acceptable salt or solvate thereof.

Embodiment 119. The method of embodiment 118, wherein androgen receptor dependent or androgen receptor mediated disease or condition is selected from benign prostate hyperplasia, hirsutism, adenomas and neoplasms of the prostate, benign or malignant tumor cells containing the androgen receptor, prostate cancer, breast cancer, endometrial cancer, and uterine cancer.

Embodiment 120. A pharmaceutical composition comprising a compound of any one of embodiments 1-102, or a pharmaceutically acceptable salt or solvate thereof, and at least one pharmaceutically acceptable excipient.

Embodiment 121. A compound, or a pharmaceutically acceptable salt or solvate thereof, that has one of the following structures:

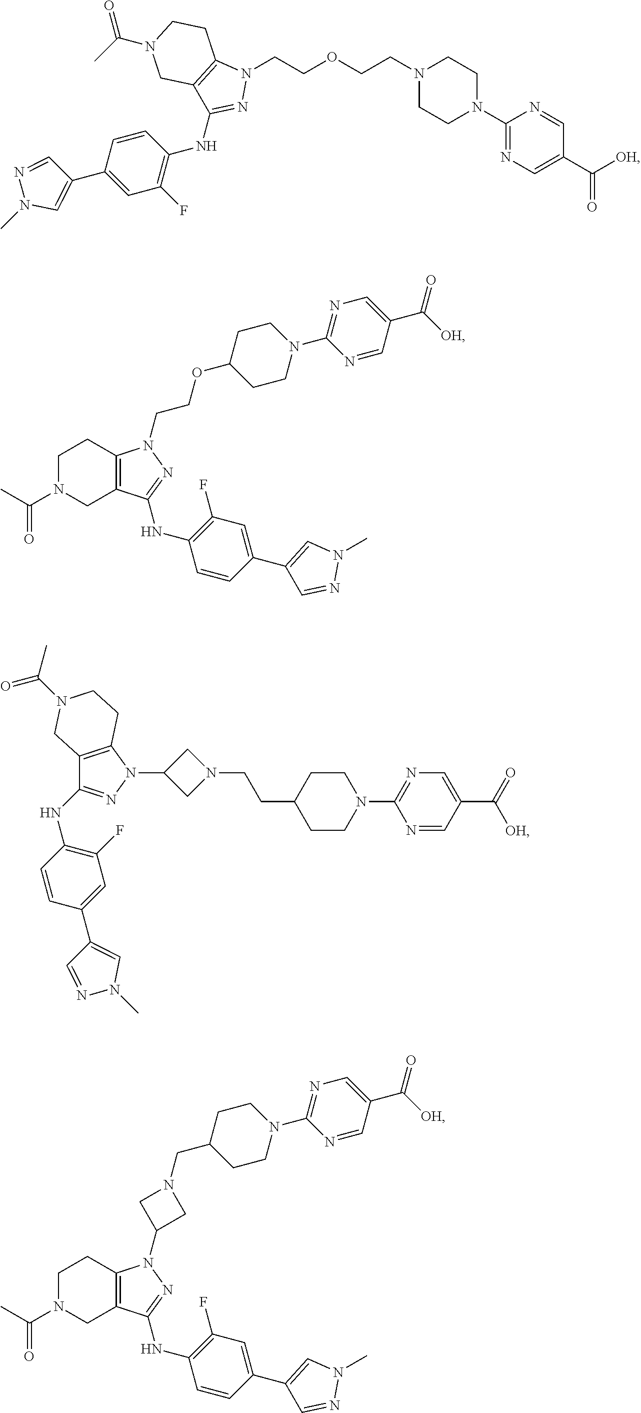 [SMILES: CC(=O)N1CCc2c(c(Nc3ccc(-c4cnn(C)c4)cc3F)nn2CCOCCN2CCN(c3ncc(C(=O)O)cn3)CC2)C1 ; CC(=O)N1CCc2c(c(Nc3ccc(-c4cnn(C)c4)cc3F)nn2CCOC2CCN(c3ncc(C(=O)O)cn3)CC2)C1 ; CC(=O)N1CCc2c(c(Nc3ccc(-c4cnn(C)c4)cc3F)nn2C2CN(CCC3CCN(c4ncc(C(=O)O)cn4)CC3)C2)C1 ; CC(=O)N1CCc2c(c(Nc3ccc(-c4cnn(C)c4)cc3F)nn2C2CN(CC3CCN(c4ncc(C(=O)O)cn4)CC3)C2)C1]

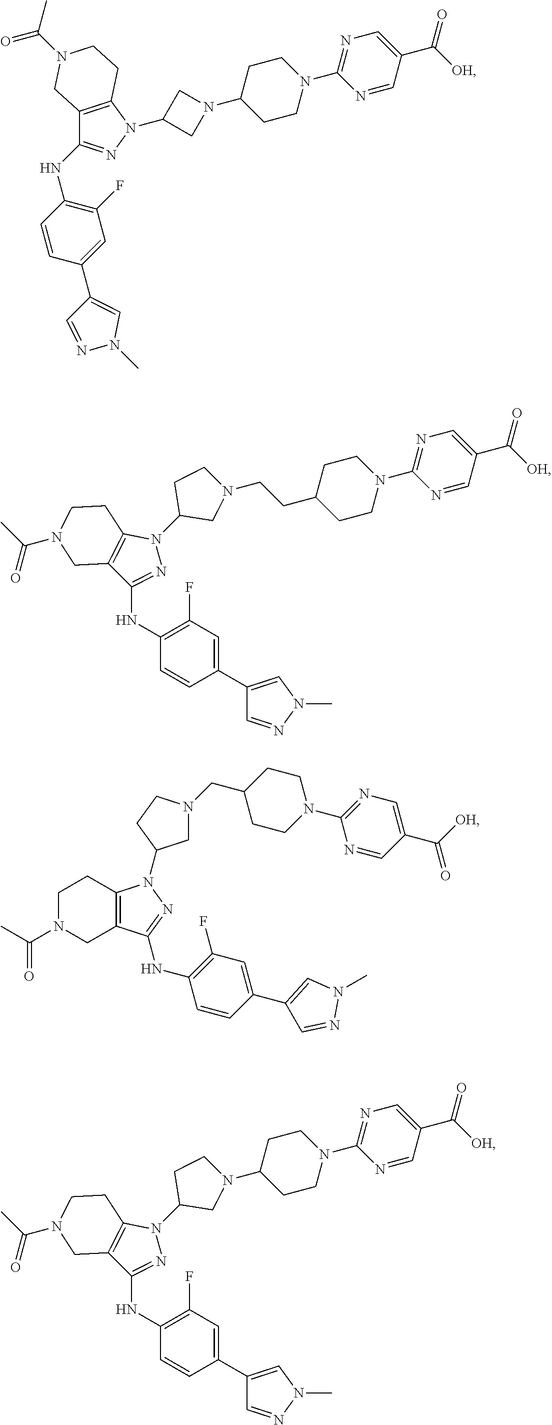

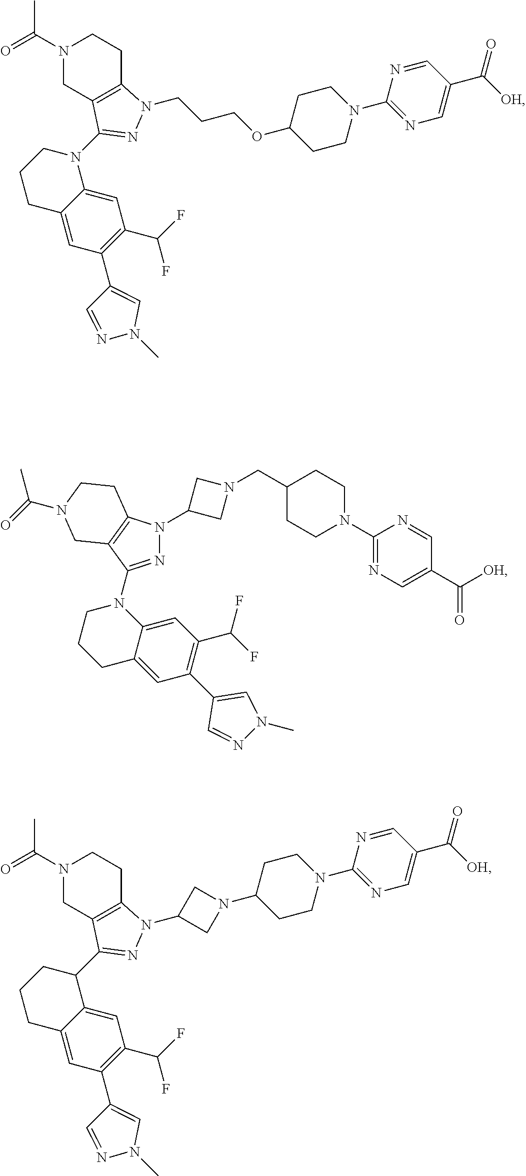 [SMILES: CC(=O)N1CCc2c(c(N3CCCc4cc(-c5cnn(C)c5)c(C(F)F)cc43)nn2CCCOC2CCN(c3ncc(C(=O)O)cn3)CC2)C1 ; CC(=O)N1CCc2c(c(N3CCCc4cc(-c5cnn(C)c5)c(C(F)F)cc43)nn2C2CN(CC3CCN(c4ncc(C(=O)O)cn4)CC3)C2)C1 ; CC(=O)N1CCc2c(c(C3CCCc4cc(-c5cnn(C)c5)c(C(F)F)cc43)nn2C2CN(C3CCN(c4ncc(C(=O)O)cn4)CC3)C2)C1]

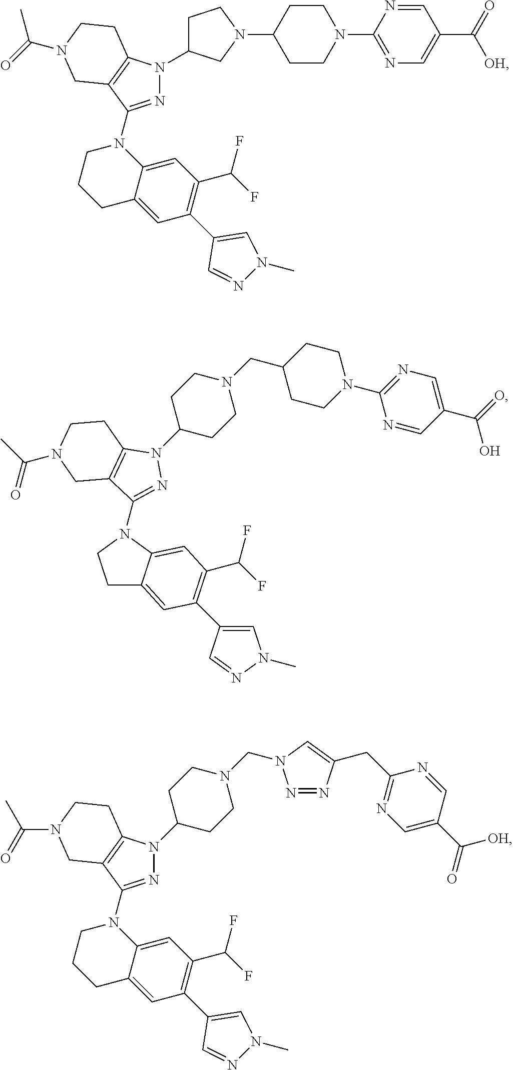 [SMILES: CC(=O)N1CCc2c(c(N3CCCc4cc(-c5cnn(C)c5)c(C(F)F)cc43)nn2C2CCN(C3CCN(c4ncc(C(=O)O)cn4)CC3)C2)C1 ; CC(=O)N1CCc2c(c(N3CCc4cc(-c5cnn(C)c5)c(C(F)F)cc43)nn2C2CCN(CC3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(Cn3cc(Cc4ncc(C(=O)O)cn4)nn3)CC2)C1]

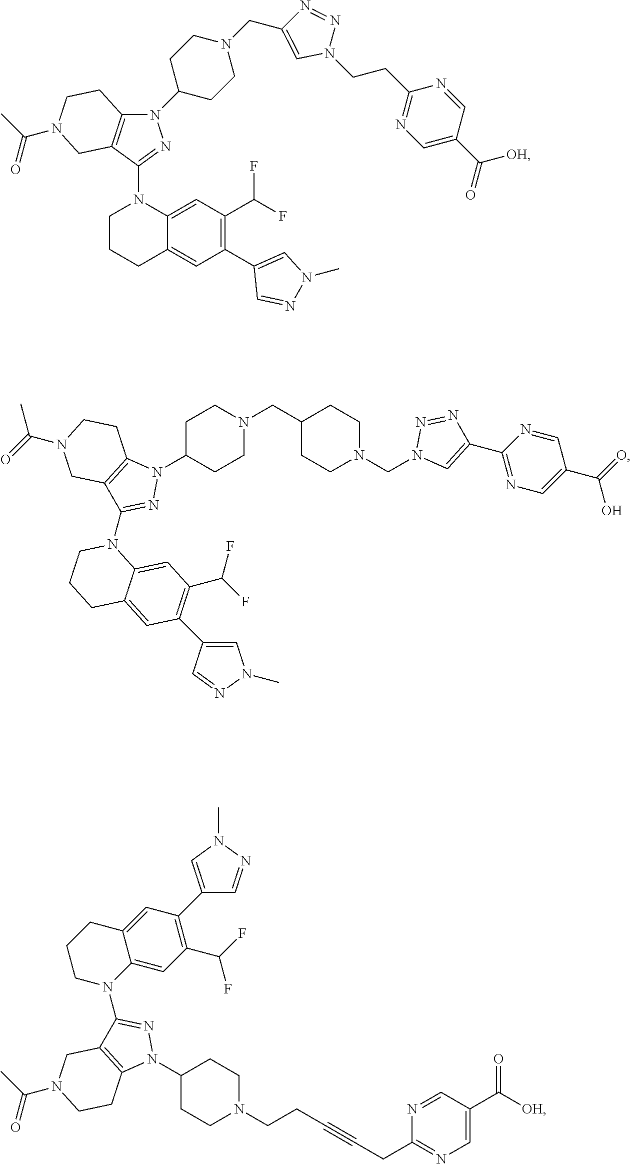 [SMILES: CC(=O)N1CCc2c(c(N3CCCc4cc(-c5cnn(C)c5)c(C(F)F)cc43)nn2C2CCN(Cc3cn(CCc4ncc(C(=O)O)cn4)nn3)CC2)C1 ; CC(=O)N1CCc2c(c(N3CCCc4cc(-c5cnn(C)c5)c(C(F)F)cc43)nn2C2CCN(CC3CCN(Cn4cc(-c5ncc(C(=O)O)cn5)nn4)CC3)CC2)C1 ; CC(=O)N1CCc2c(c(N3CCCc4cc(-c5cnn(C)c5)c(C(F)F)cc43)nn2C2CCN(CCC#CCc3ncc(C(=O)O)cn3)CC2)C1]

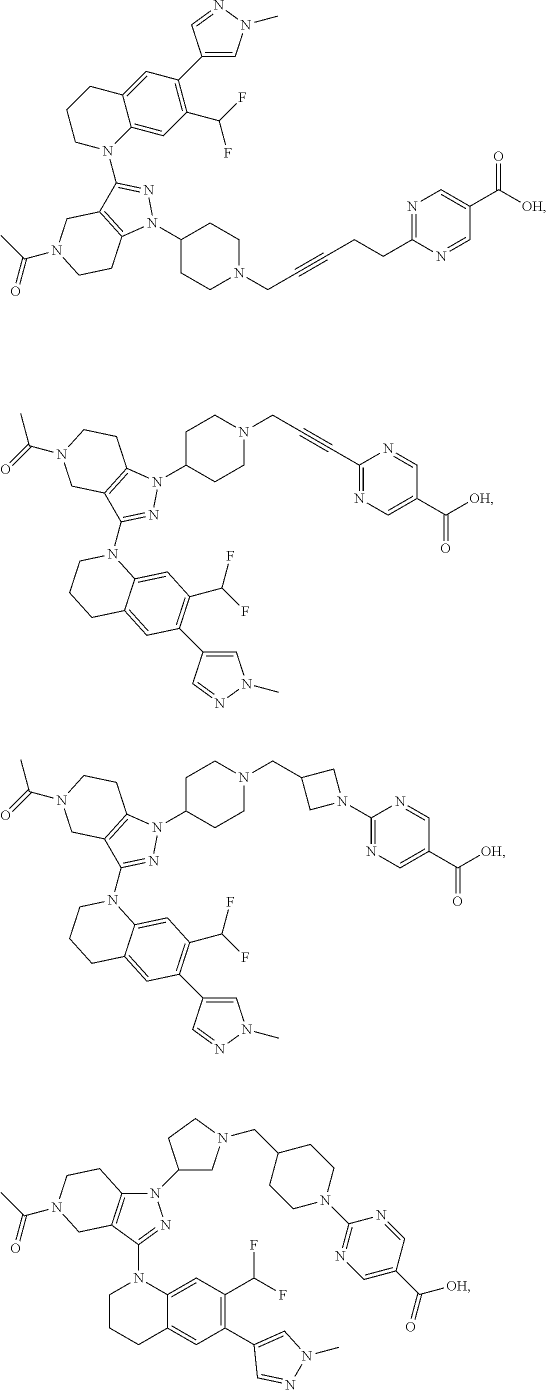 [SMILES: CC(=O)N1CCc2c(c(N3CCCc4cc(-c5cnn(C)c5)c(C(F)F)cc43)nn2C2CCN(CC#CCCc3ncc(C(=O)O)cn3)CC2)C1 ; CC(=O)N1CCc2c(c(N3CCCc4cc(-c5cnn(C)c5)c(C(F)F)cc43)nn2C2CCN(CC#Cc3ncc(C(=O)O)cn3)CC2)C1 ; CC(=O)N1CCc2c(c(N3CCCc4cc(-c5cnn(C)c5)c(C(F)F)cc43)nn2C2CCN(CC3CN(c4ncc(C(=O)O)cn4)C3)CC2)C1 ; CC(=O)N1CCc2c(c(N3CCCc4cc(-c5cnn(C)c5)c(C(F)F)cc43)nn2C2CCN(CC3CCN(c4ncc(C(=O)O)cn4)CC3)C2)C1]

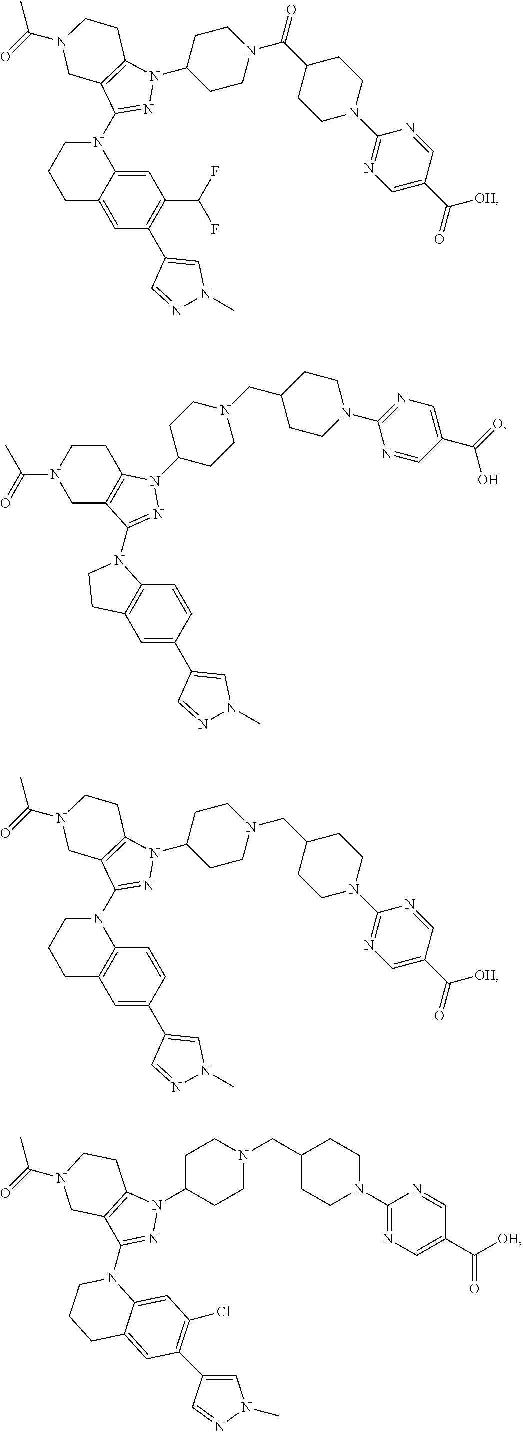 [SMILES: CC(=O)N1CCc2c(c(N3CCCc4cc(-c5cnn(C)c5)c(C(F)F)cc43)nn2C2CCN(C(=O)C3CCN(c4ncc(C(=O)O)cn4)CC3)CC2)C1 ; CC(=O)N1CCc2c(c(N3CCc4cc(-c5cnn(C)c5)ccc43)nn2C2CCN(CC3CCN(c4ncc(C(=O)O)cn4)CC3)CC2)C1 ; CC(=O)N1CCc2c(c(N3CCCc4cc(-c5cnn(C)c5)ccc43)nn2C2CCN(CC3CCN(c4ncc(C(=O)O)cn4)CC3)CC2)C1 ; CC(=O)N1CCc2c(c(N3CCCc4cc(-c5cnn(C)c5)c(Cl)cc43)nn2C2CCN(CC3CCN(c4ncc(C(=O)O)cn4)CC3)CC2)C1]

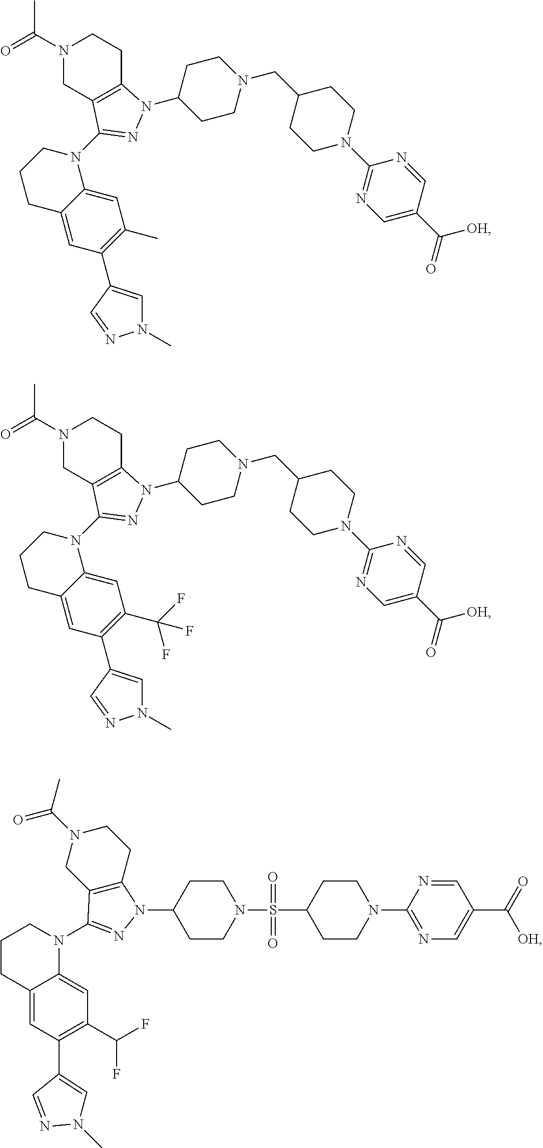 [SMILES: CC(=O)N1CCc2c(c(N3CCCc4cc(-c5cnn(C)c5)c(C)cc43)nn2C2CCN(CC3CCN(c4ncc(C(=O)O)cn4)CC3)CC2)C1 ; CC(=O)N1CCc2c(c(N3CCCc4cc(-c5cnn(C)c5)c(C(F)(F)F)cc43)nn2C2CCN(CC3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(S(=O)(=O)C3CCN(c4ncc(C(=O)O)cn4)CC3)CC2)C1]

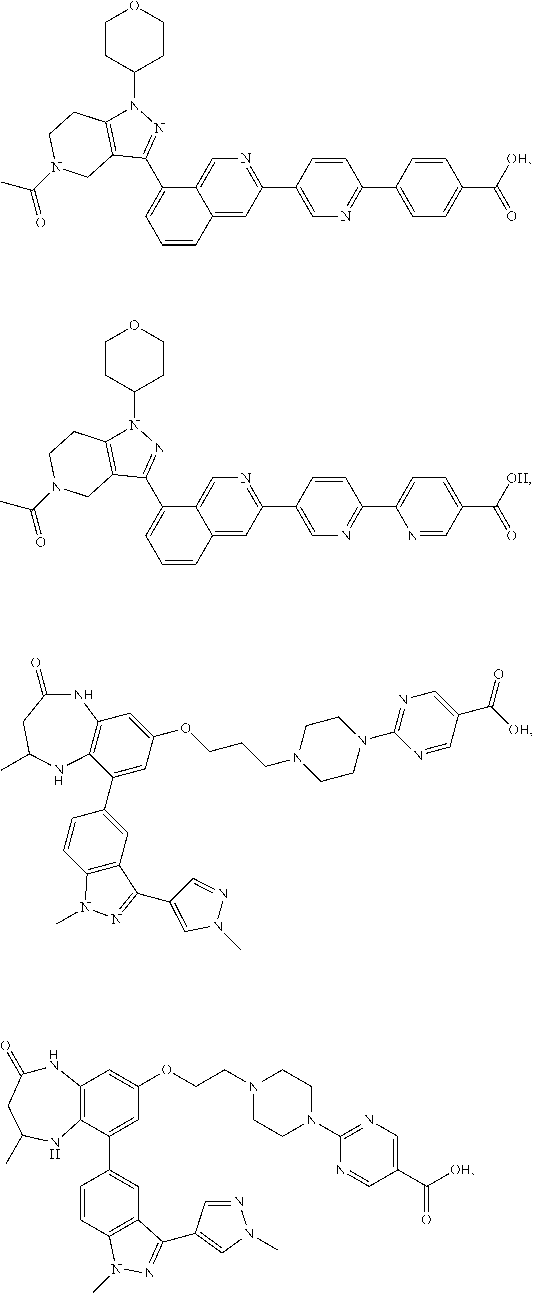 [SMILES: CC(=O)N1CCc2c(c(-c3cccc4cc(-c5ccc(-c6ccc(C(=O)O)cc6)nc5)ncc34)nn2C2CCOCC2)C1 ; CC(=O)N1CCc2c(c(-c3cccc4cc(-c5ccc(-c6ccc(C(=O)O)cn6)nc5)ncc34)nn2C2CCOCC2)C1 ; CC1CC(=O)Nc2cc(OCCCN3CCN(c4ncc(C(=O)O)cn4)CC3)cc(-c3ccc4c(c3)c(-c3cnn(C)c3)nn4C)c2N1 ; CC1CC(=O)Nc2cc(OCCN3CCN(c4ncc(C(=O)O)cn4)CC3)cc(-c3ccc4c(c3)c(-c3cnn(C)c3)nn4C)c2N1]

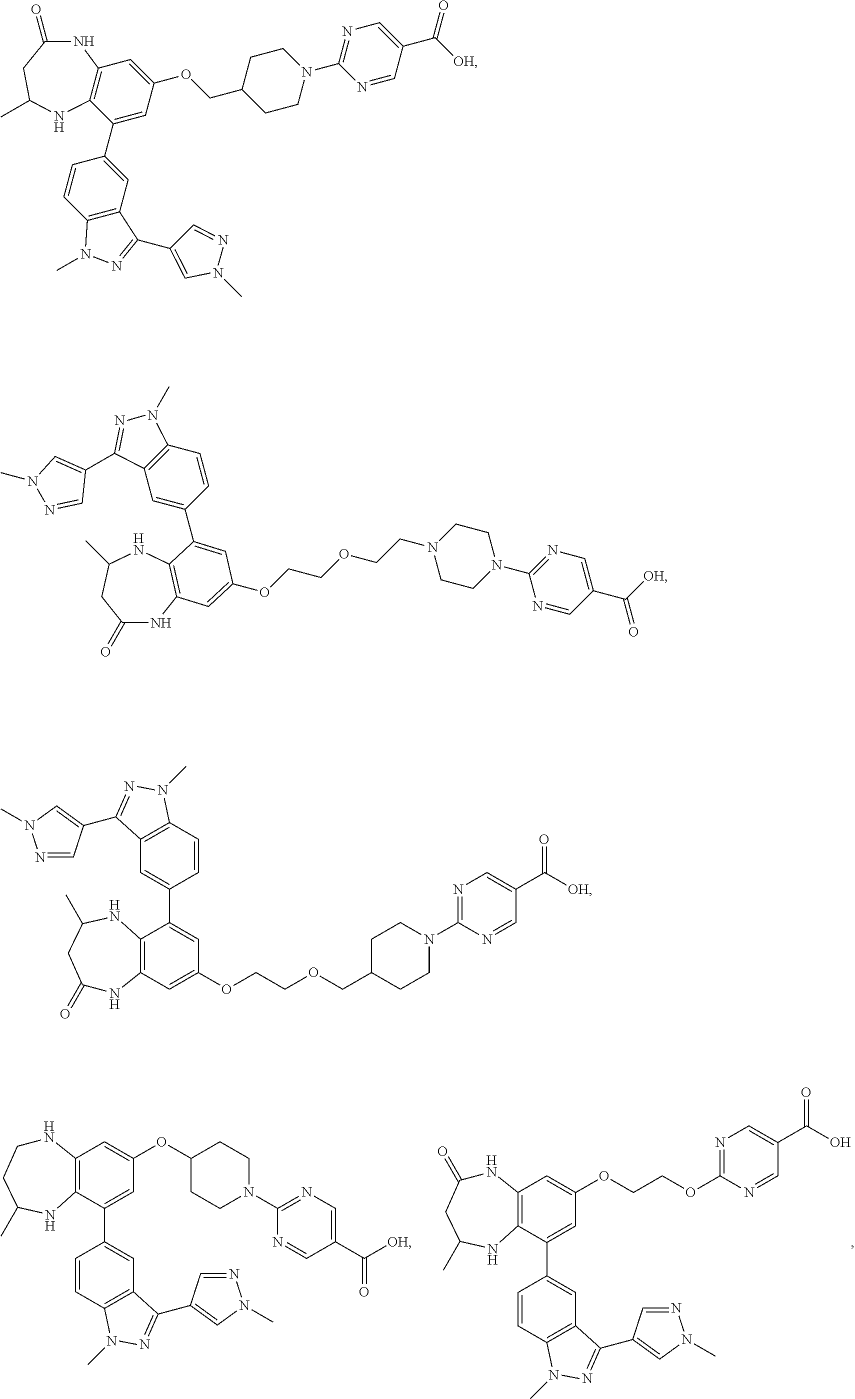 [SMILES: CC1CC(=O)Nc2cc(OCC3CCN(c4ncc(C(=O)O)cn4)CC3)cc(-c3ccc4c(c3)c(-c3cnn(C)c3)nn4C)c2N1 ; CC1CC(=O)Nc2cc(OCCOCCN3CCN(c4ncc(C(=O)O)cn4)CC3)cc(-c3ccc4c(c3)c(-c3cnn(C)c3)nn4C)c2N1 ; CC1CC(=O)Nc2cc(OCCOCC3CCN(c4ncc(C(=O)O)cn4)CC3)cc(-c3ccc4c(c3)c(-c3cnn(C)c3)nn4C)c2N1 ; CC1CCNc2cc(OC3CCN(c4ncc(C(=O)O)cn4)CC3)cc(-c3ccc4c(c3)c(-c3cnn(C)c3)nn4C)c2N1 ; CC1CC(=O)Nc2cc(OCCOc3ncc(C(=O)O)cn3)cc(-c3ccc4c(c3)c(-c3cnn(C)c3)nn4C)c2N1]

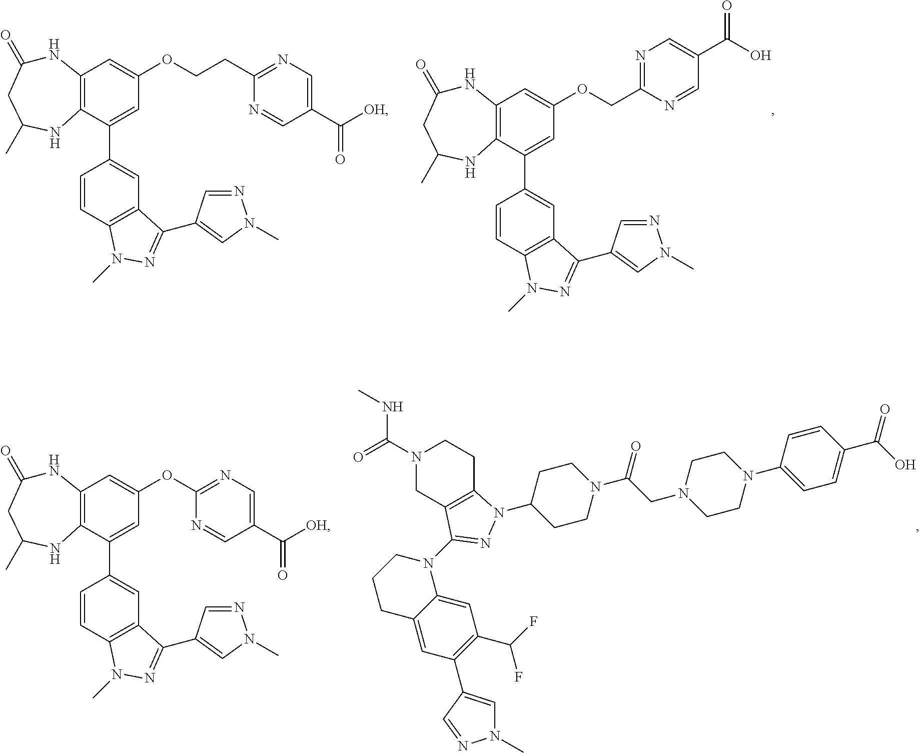 [SMILES: CC1CC(=O)Nc2cc(OCCc3ncc(C(=O)O)cn3)cc(-c3ccc4c(c3)c(-c3cnn(C)c3)nn4C)c2N1 ; CC1CC(=O)Nc2cc(OCc3ncc(C(=O)O)cn3)cc(-c3ccc4c(c3)c(-c3cnn(C)c3)nn4C)c2N1 ; CC1CC(=O)Nc2cc(Oc3ncc(C(=O)O)cn3)cc(-c3ccc4c(c3)c(-c3cnn(C)c3)nn4C)c2N1 ; CNC(=O)N1CCc2c(c(N3CCCc4cc(-c5cnn(C)c5)c(C(F)F)cc43)nn2C2CCN(C(=O)CN3CCN(c4ccc(C(=O)O)cc4)CC3)CC2)C1]

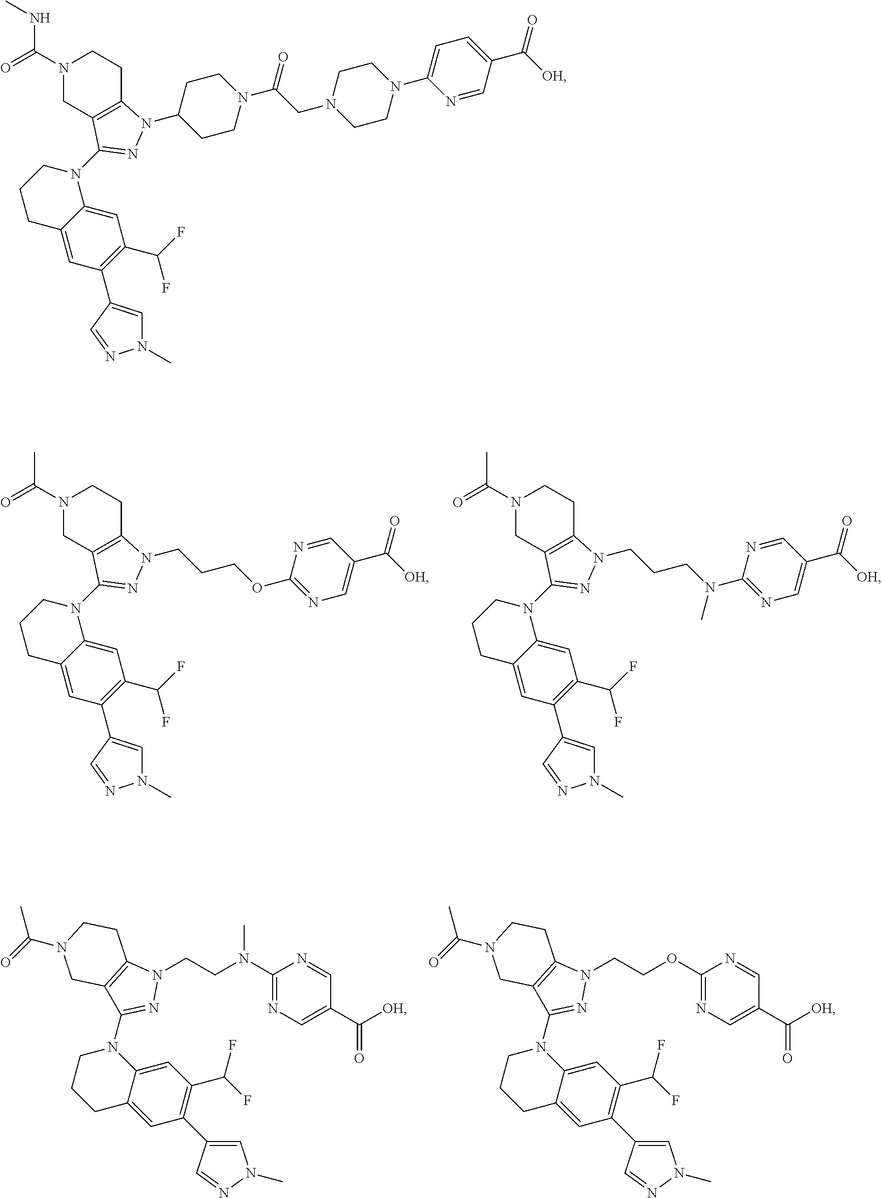 [SMILES: CNC(=O)N1CCc2c(c(N3CCCc4cc(-c5cnn(C)c5)c(C(F)F)cc43)nn2C2CCN(C(=O)CN3CCN(c4ccc(C(=O)O)cn4)CC3)CC2)C1 ; CC(=O)N1CCc2c(c(N3CCCc4cc(-c5cnn(C)c5)c(C(F)F)cc43)nn2CCCOc2ncc(C(=O)O)cn2)C1 ; CC(=O)N1CCc2c(c(N3CCCc4cc(-c5cnn(C)c5)c(C(F)F)cc43)nn2CCCN(C)c2ncc(C(=O)O)cn2)C1 ; CC(=O)N1CCc2c(c(N3CCCc4cc(-c5cnn(C)c5)c(C(F)F)cc43)nn2CCN(C)c2ncc(C(=O)O)cn2)C1 ; CC(=O)N1CCc2c(c(N3CCCc4cc(-c5cnn(C)c5)c(C(F)F)cc43)nn2CCOc2ncc(C(=O)O)cn2)C1]

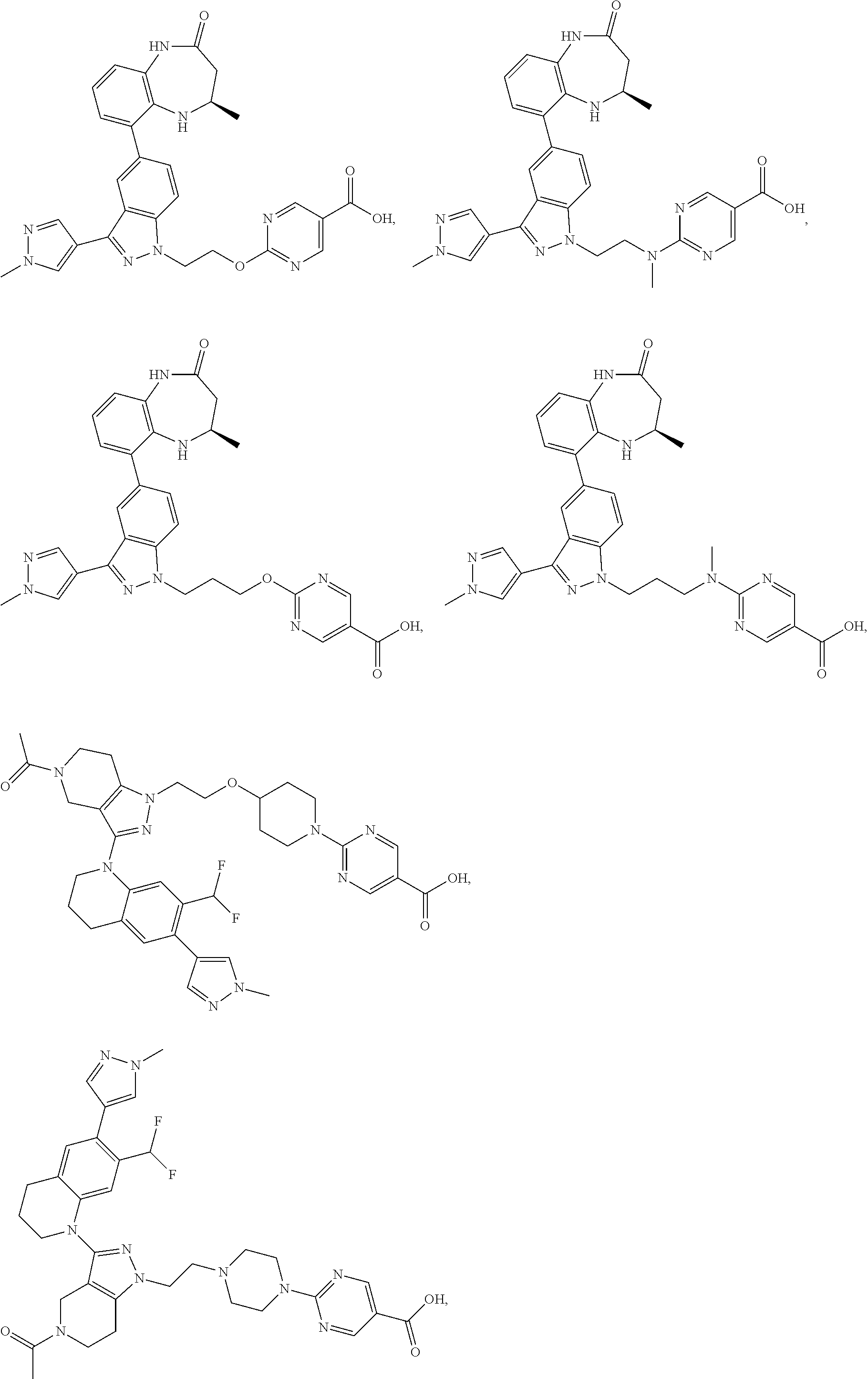 [SMILES: C[C@@H]1CC(=O)Nc2cccc(-c3ccc4c(c3)c(-c3cnn(C)c3)nn4CCOc3ncc(C(=O)O)cn3)c2N1 ; C[C@@H]1CC(=O)Nc2cccc(-c3ccc4c(c3)c(-c3cnn(C)c3)nn4CCN(C)c3ncc(C(=O)O)cn3)c2N1 ; C[C@@H]1CC(=O)Nc2cccc(-c3ccc4c(c3)c(-c3cnn(C)c3)nn4CCCOc3ncc(C(=O)O)cn3)c2N1 ; C[C@@H]1CC(=O)Nc2cccc(-c3ccc4c(c3)c(-c3cnn(C)c3)nn4CCCN(C)c3ncc(C(=O)O)cn3)c2N1 ; CC(=O)N1CCc2c(c(N3CCCc4cc(-c5cnn(C)c5)c(C(F)F)cc43)nn2CCOC2CCN(c3ncc(C(=O)O)cn3)CC2)C1 ; CC(=O)N1CCc2c(c(N3CCCc4cc(-c5cnn(C)c5)c(C(F)F)cc43)nn2CCN2CCN(c3ncc(C(=O)O)cn3)CC2)C1]

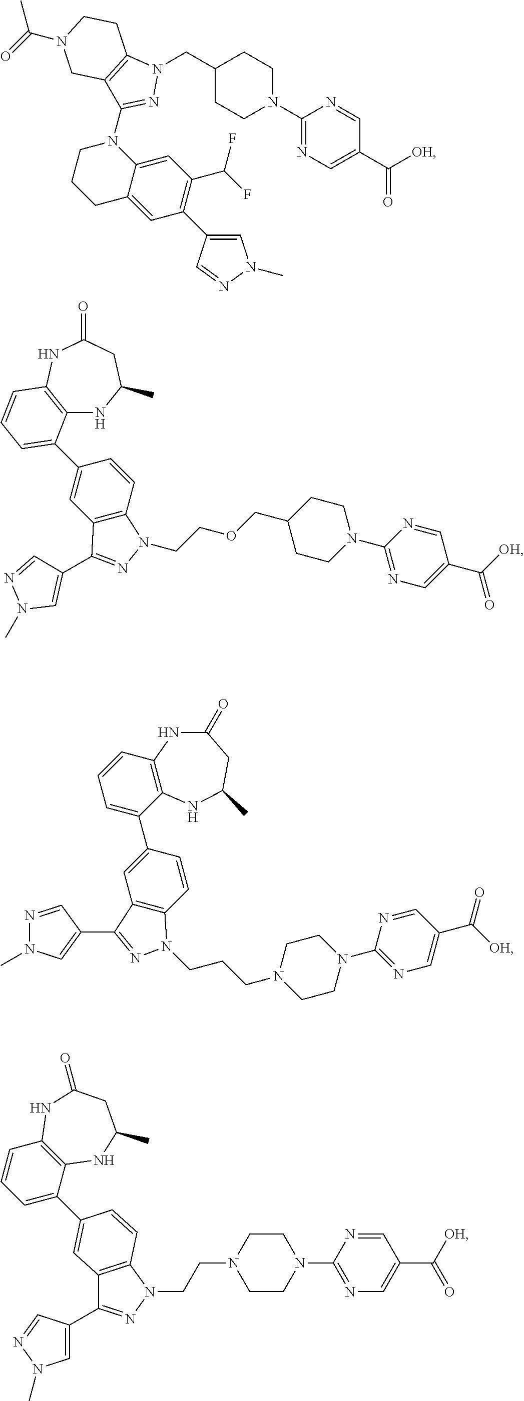 [SMILES: CC(=O)N1CCc2c(c(N3CCCc4cc(-c5cnn(C)c5)c(C(F)F)cc43)nn2CC2CCN(c3ncc(C(=O)O)cn3)CC2)C1 ; C[C@@H]1CC(=O)Nc2cccc(-c3ccc4c(c3)c(-c3cnn(C)c3)nn4CCOCC3CCN(c4ncc(C(=O)O)cn4)CC3)c2N1 ; C[C@@H]1CC(=O)Nc2cccc(-c3ccc4c(c3)c(-c3cnn(C)c3)nn4CCCN3CCN(c4ncc(C(=O)O)cn4)CC3)c2N1 ; C[C@@H]1CC(=O)Nc2cccc(-c3ccc4c(c3)c(-c3cnn(C)c3)nn4CCN3CCN(c4ncc(C(=O)O)cn4)CC3)c2N1]

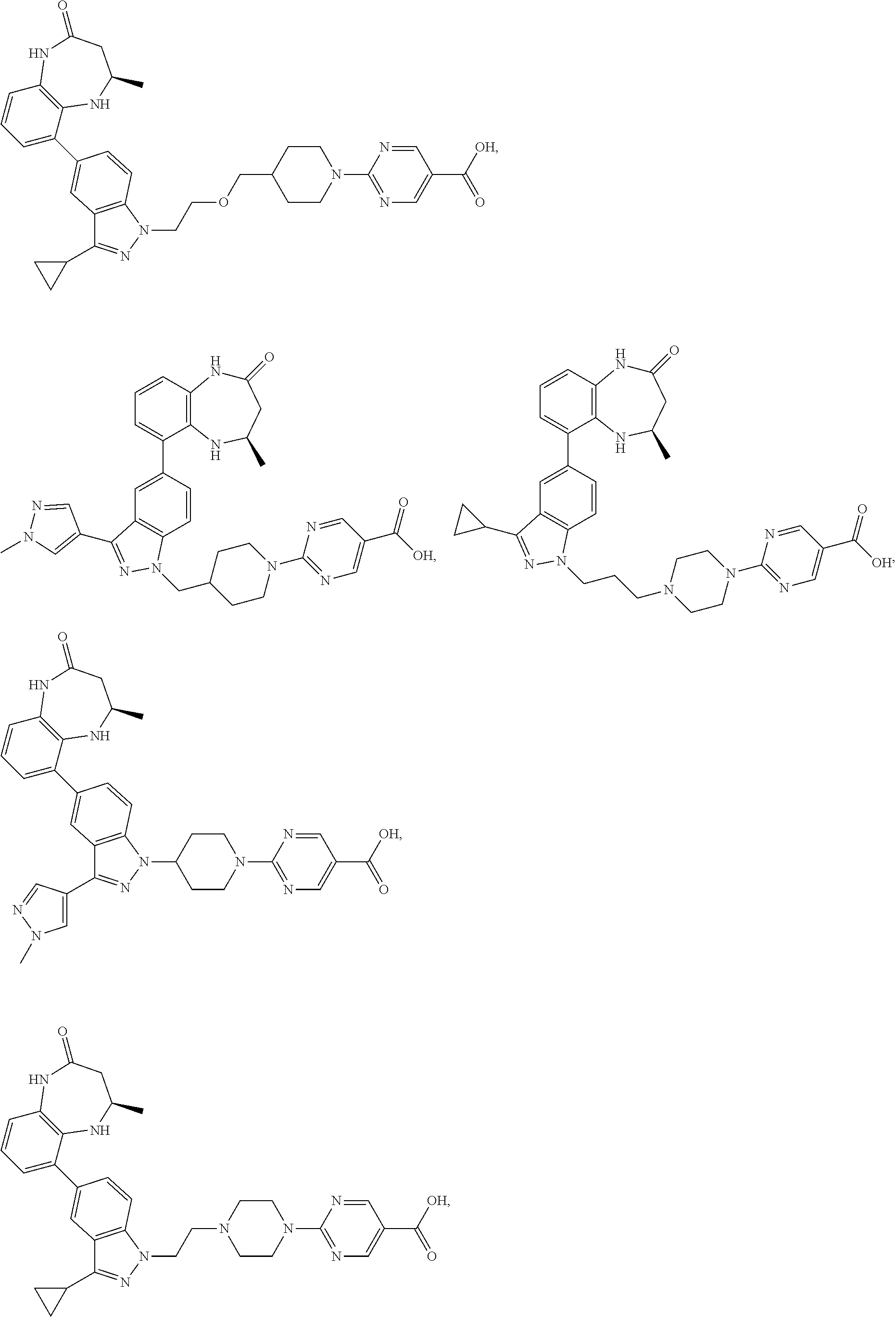

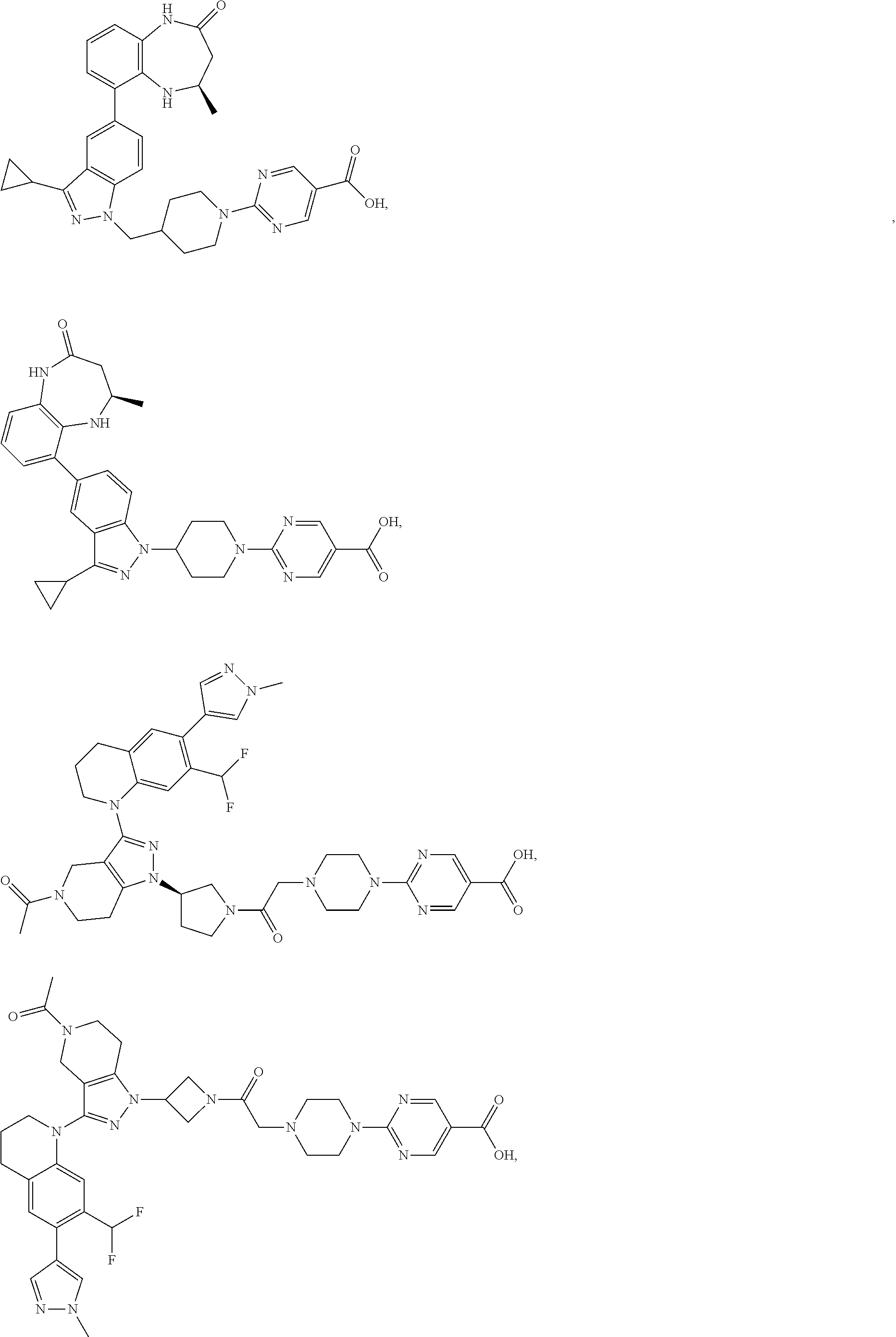 [SMILES: C[C@@H]1CC(=O)Nc2cccc(-c3ccc4c(c3)c(C3CC3)nn4CC3CCN(c4ncc(C(=O)O)cn4)CC3)c2N1 ; C[C@@H]1CC(=O)Nc2cccc(-c3ccc4c(c3)c(C3CC3)nn4C3CCN(c4ncc(C(=O)O)cn4)CC3)c2N1 ; CC(=O)N1CCc2c(c(N3CCCc4cc(-c5cnn(C)c5)c(C(F)F)cc43)nn2[C@@H]2CCN(C(=O)CN3CCN(c4ncc(C(=O)O)cn4)CC3)C2)C1 ; CC(=O)N1CCc2c(c(N3CCCc4cc(-c5cnn(C)c5)c(C(F)F)cc43)nn2C2CN(C(=O)CN3CCN(c4ncc(C(=O)O)cn4)CC3)C2)C1]

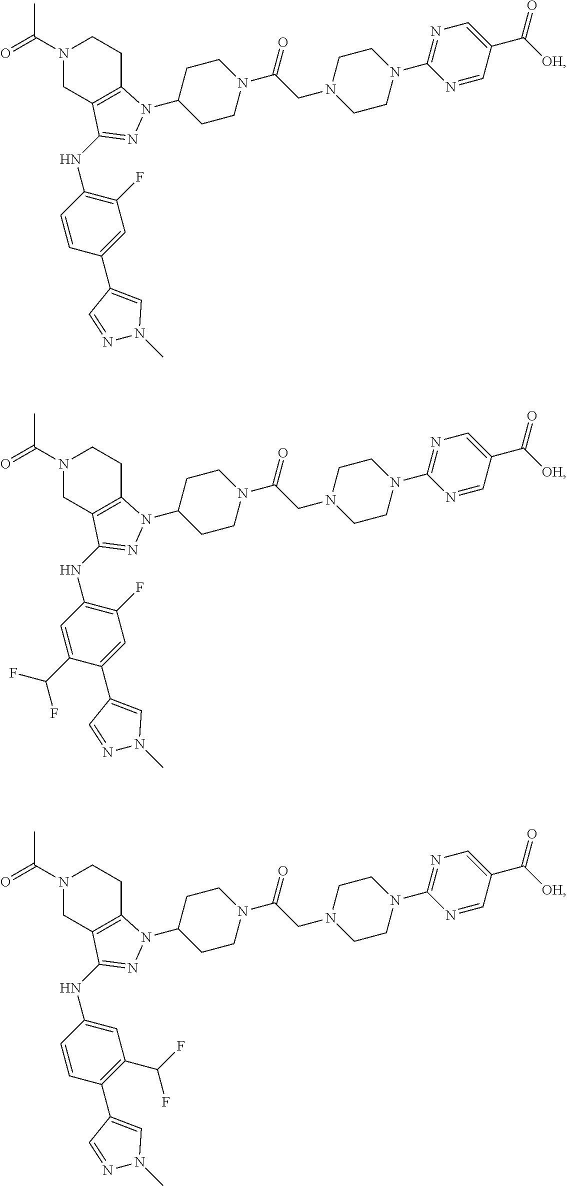 [SMILES: CC(=O)N1CCc2c(c(Nc3ccc(-c4cnn(C)c4)cc3F)nn2C2CCN(C(=O)CN3CCN(c4ncc(C(=O)O)cn4)CC3)CC2)C1 ; CC(=O)N1CCc2c(c(Nc3cc(C(F)F)c(-c4cnn(C)c4)cc3F)nn2C2CCN(C(=O)CN3CCN(c4ncc(C(=O)O)cn4)CC3)CC2)C1 ; CC(=O)N1CCc2c(c(Nc3ccc(-c4cnn(C)c4)c(C(F)F)c3)nn2C2CCN(C(=O)CN3CCN(c4ncc(C(=O)O)cn4)CC3)CC2)C1]

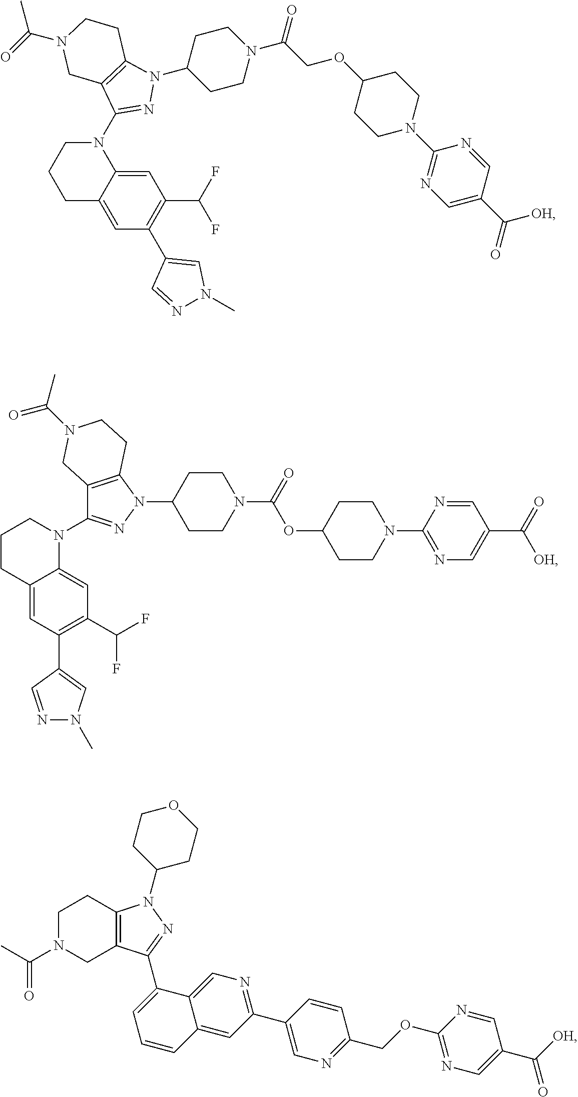 [SMILES: CC(=O)N1CCc2c(c(N3CCCc4cc(-c5cnn(C)c5)c(C(F)F)cc43)nn2C2CCN(C(=O)COC3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(C(=O)OC3CCN(c4ncc(C(=O)O)cn4)CC3)CC2)C1 ; CC(=O)N1CCc2c(c(-c3cccc4cc(-c5ccc(COc6ncc(C(=O)O)cn6)nc5)ncc34)nn2C2CCOCC2)C1]

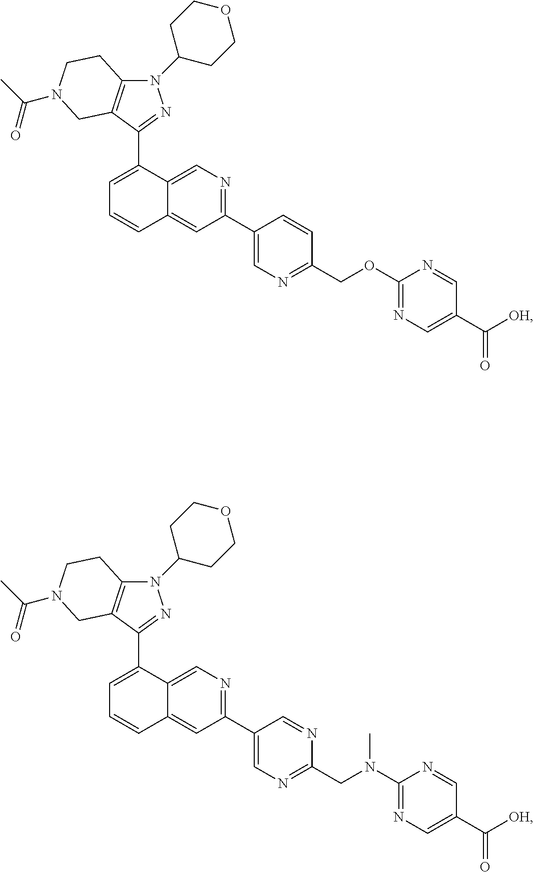 [SMILES: CC(=O)N1CCc2c(c(-c3cccc4cc(-c5ccc(COc6ncc(C(=O)O)cn6)nc5)ncc34)nn2C2CCOCC2)C1 ; CC(=O)N1CCc2c(c(-c3cccc4cc(-c5cnc(CN(C)c6ncc(C(=O)O)cn6)nc5)ncc34)nn2C2CCOCC2)C1]

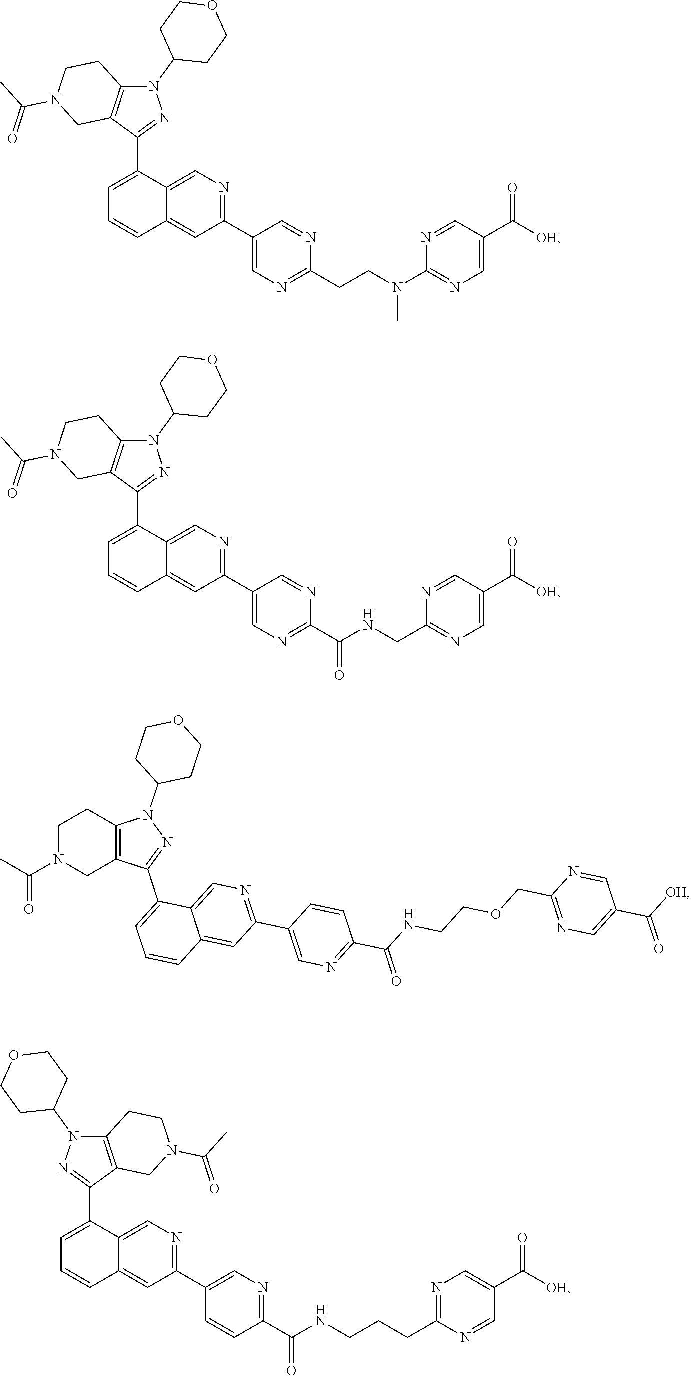 [SMILES: CC(=O)N1CCc2c(c(-c3cccc4cc(-c5cnc(CCN(C)c6ncc(C(=O)O)cn6)nc5)ncc34)nn2C2CCOCC2)C1 ; CC(=O)N1CCc2c(c(-c3cccc4cc(-c5cnc(C(=O)NCc6ncc(C(=O)O)cn6)nc5)ncc34)nn2C2CCOCC2)C1 ; CC(=O)N1CCc2c(c(-c3cccc4cc(-c5ccc(C(=O)NCCOCc6ncc(C(=O)O)cn6)nc5)ncc34)nn2C2CCOCC2)C1 ; CC(=O)N1CCc2c(c(-c3cccc4cc(-c5ccc(C(=O)NCCCc6ncc(C(=O)O)cn6)nc5)ncc34)nn2C2CCOCC2)C1]

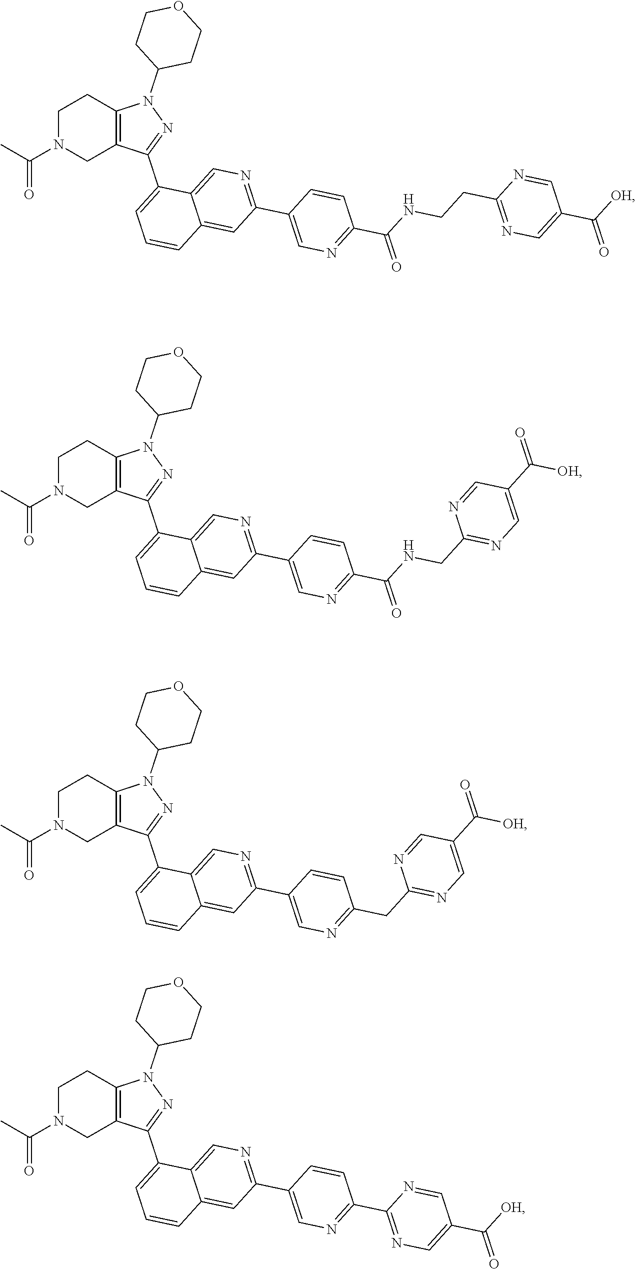 [SMILES: CC(=O)N1CCc2c(c(-c3cccc4cc(-c5ccc(C(=O)NCCc6ncc(C(=O)O)cn6)nc5)ncc34)nn2C2CCOCC2)C1 ; CC(=O)N1CCc2c(c(-c3cccc4cc(-c5ccc(C(=O)NCc6ncc(C(=O)O)cn6)nc5)ncc34)nn2C2CCOCC2)C1 ; CC(=O)N1CCc2c(c(-c3cccc4cc(-c5ccc(Cc6ncc(C(=O)O)cn6)nc5)ncc34)nn2C2CCOCC2)C1 ; CC(=O)N1CCc2c(c(-c3cccc4cc(-c5ccc(-c6ncc(C(=O)O)cn6)nc5)ncc34)nn2C2CCOCC2)C1]

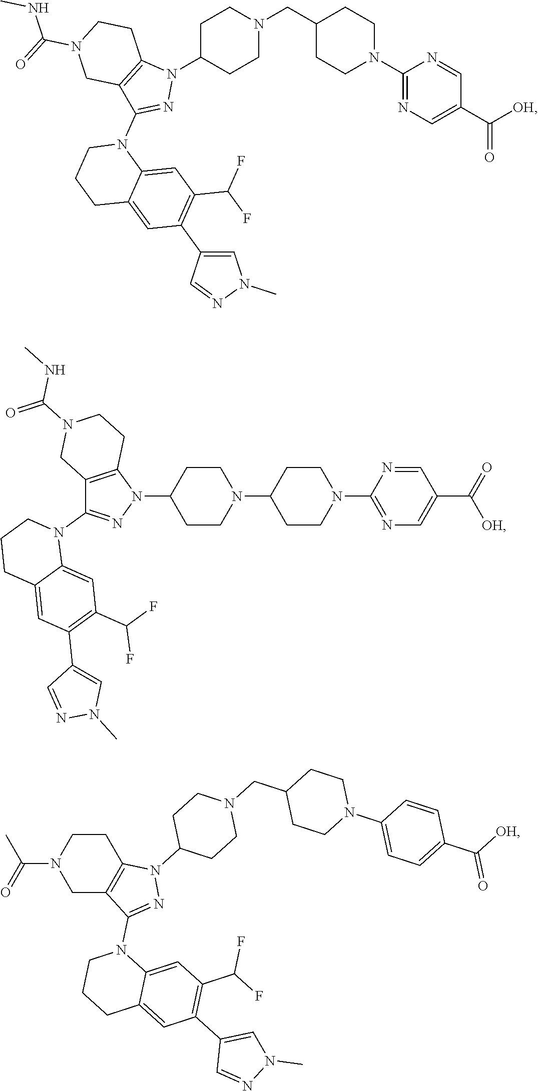 [SMILES: CNC(=O)N1CCc2c(c(N3CCCc4cc(-c5cnn(C)c5)c(C(F)F)cc43)nn2C2CCN(CC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(CC3CCN(c4ccc(C(=O)O)cc4)CC3)CC2)C1]

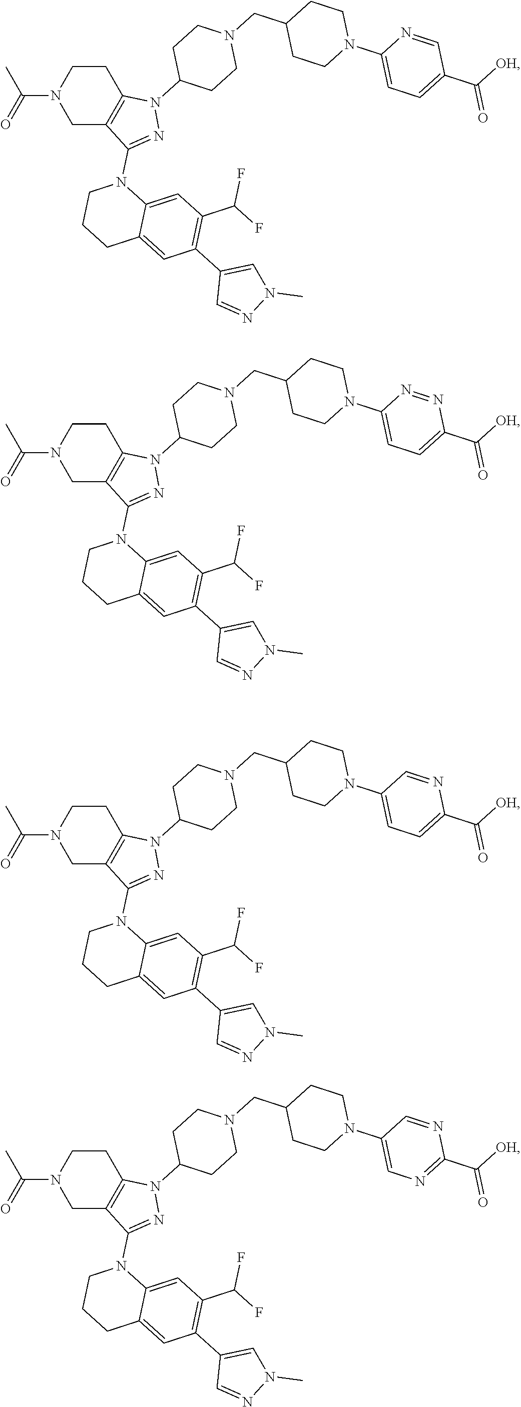

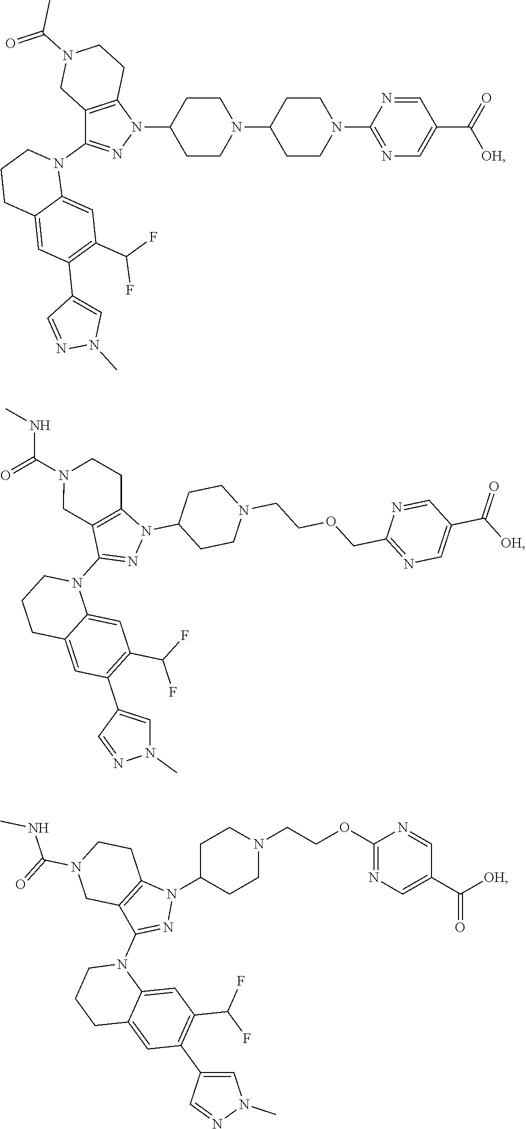 [SMILES: CC(=O)N1CCc2c(c(N3CCCc4cc(-c5cnn(C)c5)c(C(F)F)cc43)nn2C2CCN(C3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(CCOCc3ncc(C(=O)O)cn3)CC2)C1 ; CNC(=O)N1CCc2c(c(N3CCCc4cc(-c5cnn(C)c5)c(C(F)F)cc43)nn2C2CCN(CCOc3ncc(C(=O)O)cn3)CC2)C1]

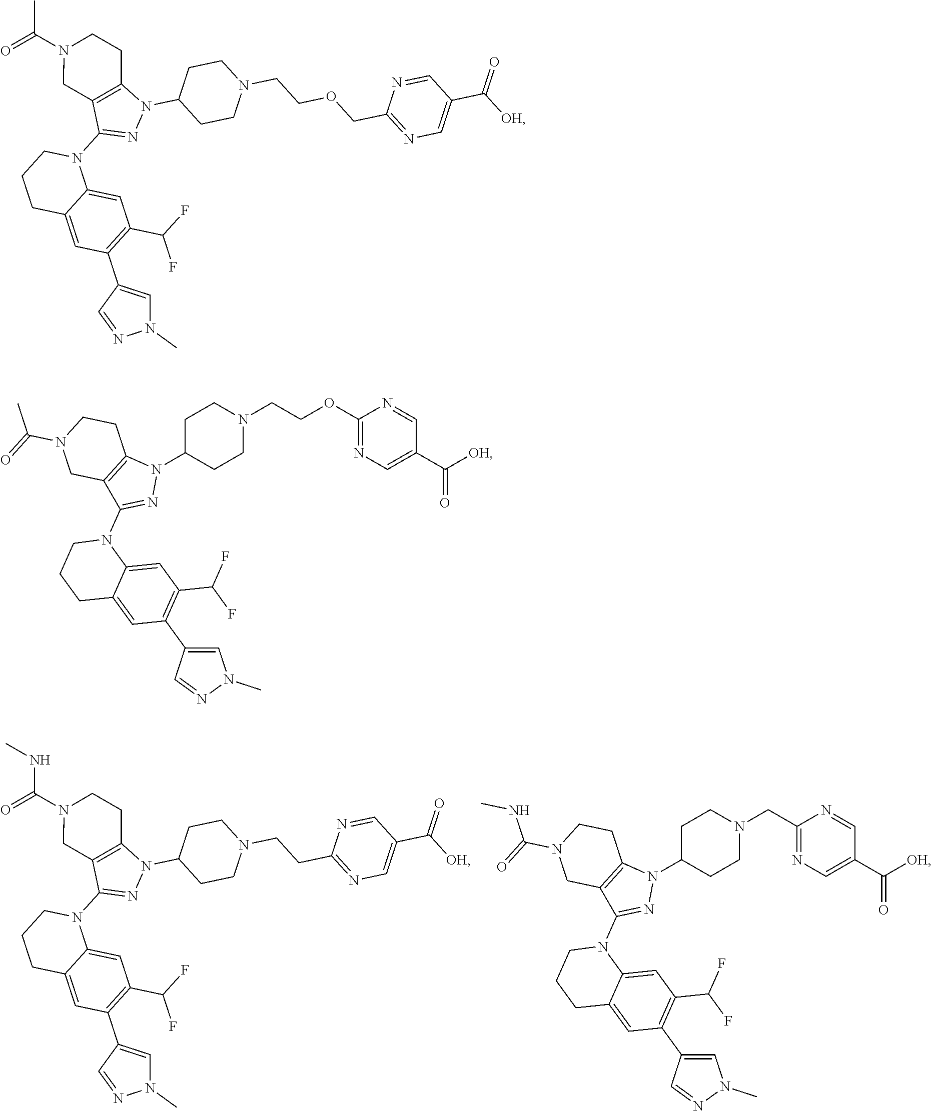

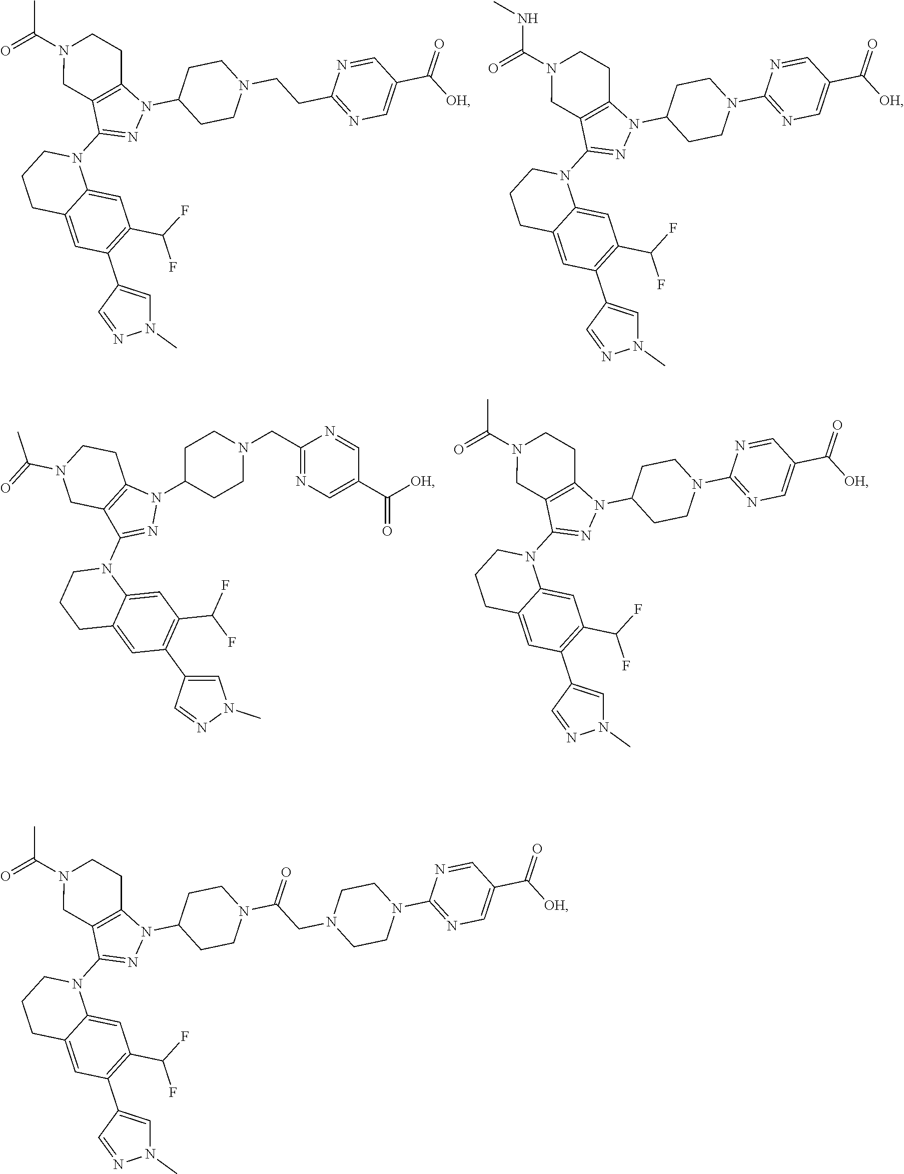 [SMILES: CC(=O)N1CCc2c(c(N3CCCc4cc(-c5cnn(C)c5)c(C(F)F)cc43)nn2C2CCN(CCc3ncc(C(=O)O)cn3)CC2)C1 ; CNC(=O)N1CCc2c(c(N3CCCc4cc(-c5cnn(C)c5)c(C(F)F)cc43)nn2C2CCN(c3ncc(C(=O)O)cn3)CC2)C1 ; CC(=O)N1CCc2c(c(N3CCCc4cc(-c5cnn(C)c5)c(C(F)F)cc43)nn2C2CCN(Cc3ncc(C(=O)O)cn3)CC2)C1 ; CC(=O)N1CCc2c(c(N3CCCc4cc(-c5cnn(C)c5)c(C(F)F)cc43)nn2C2CCN(c3ncc(C(=O)O)cn3)CC2)C1 ; CC(=O)N1CCc2c(c(N3CCCc4cc(-c5cnn(C)c5)c(C(F)F)cc43)nn2C2CCN(C(=O)CN3CCN(c4ncc(C(=O)O)cn4)CC3)CC2)C1]

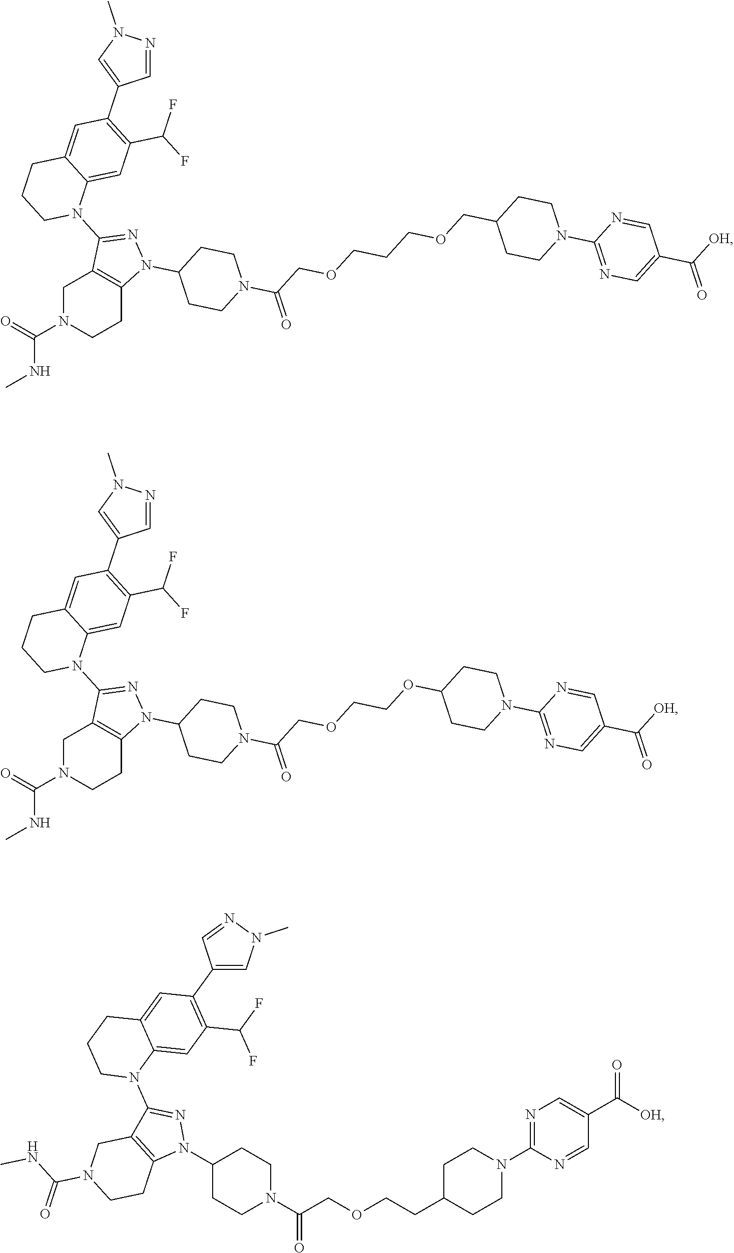 [SMILES: CNC(=O)N1CCc2c(c(N3CCCc4cc(-c5cnn(C)c5)c(C(F)F)cc43)nn2C2CCN(C(=O)COCCCOCC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)COCCOC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)COCCC3CCN(c4ncc(C(=O)O)cn4)CC3)CC2)C1]

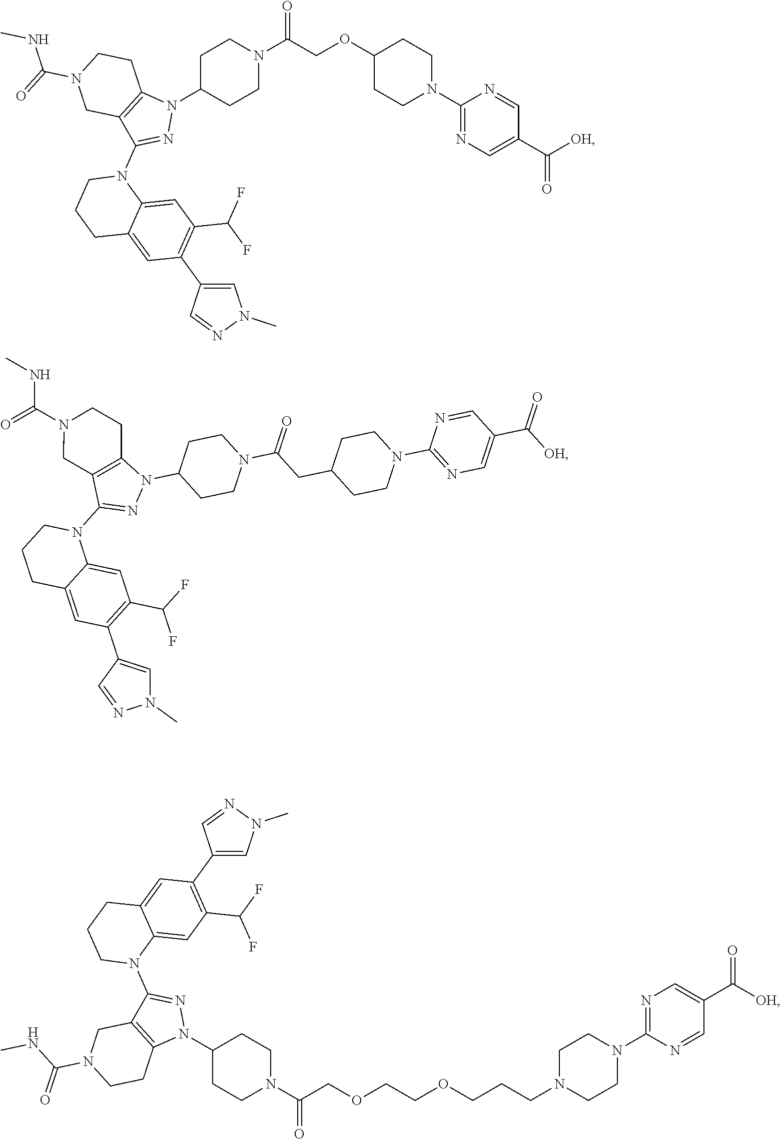 [SMILES: CNC(=O)N1CCc2c(c(N3CCCc4cc(-c5cnn(C)c5)c(C(F)F)cc43)nn2C2CCN(C(=O)COC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)CC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)COCCOCCCN3CCN(c4ncc(C(=O)O)cn4)CC3)CC2)C1]

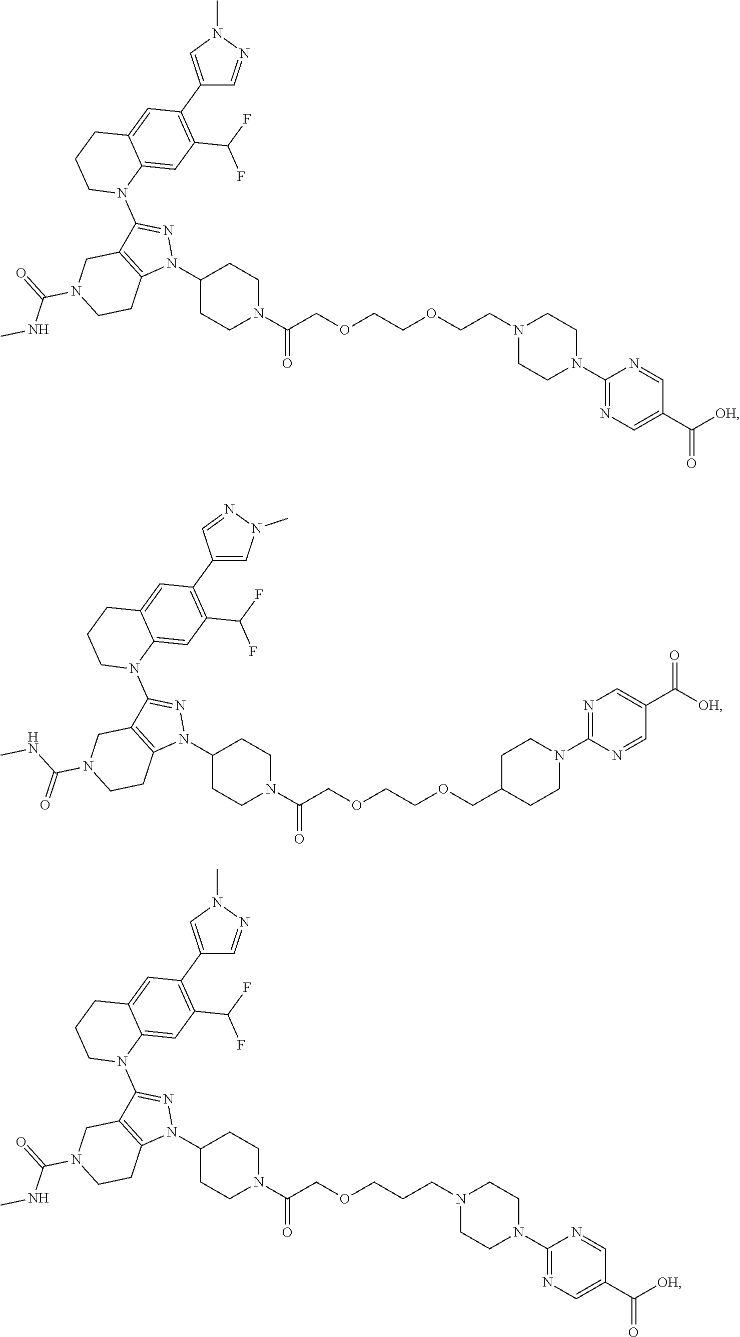 [SMILES: CNC(=O)N1CCc2c(c(N3CCCc4cc(-c5cnn(C)c5)c(C(F)F)cc43)nn2C2CCN(C(=O)COCCOCCN3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)COCCOCC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)COCCCN3CCN(c4ncc(C(=O)O)cn4)CC3)CC2)C1]

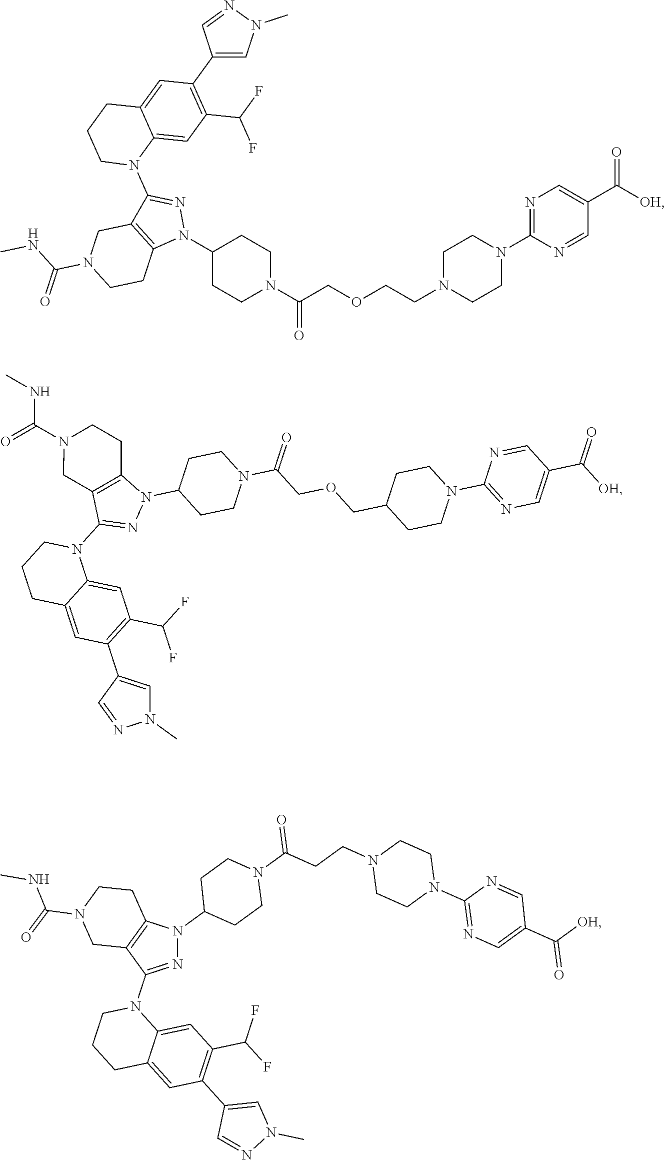 [SMILES: CNC(=O)N1CCc2c(c(N3CCCc4cc(-c5cnn(C)c5)c(C(F)F)cc43)nn2C2CCN(C(=O)COCCN3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)COCC3CCN(c4ncc(C(=O)O)cn4)CC3)CC2)C1 ; CNC(=O)N1CCc2c(c(N3CCCc4cc(-c5cnn(C)c5)c(C(F)F)cc43)nn2C2CCN(C(=O)CCN3CCN(c4ncc(C(=O)O)cn4)CC3)CC2)C1]

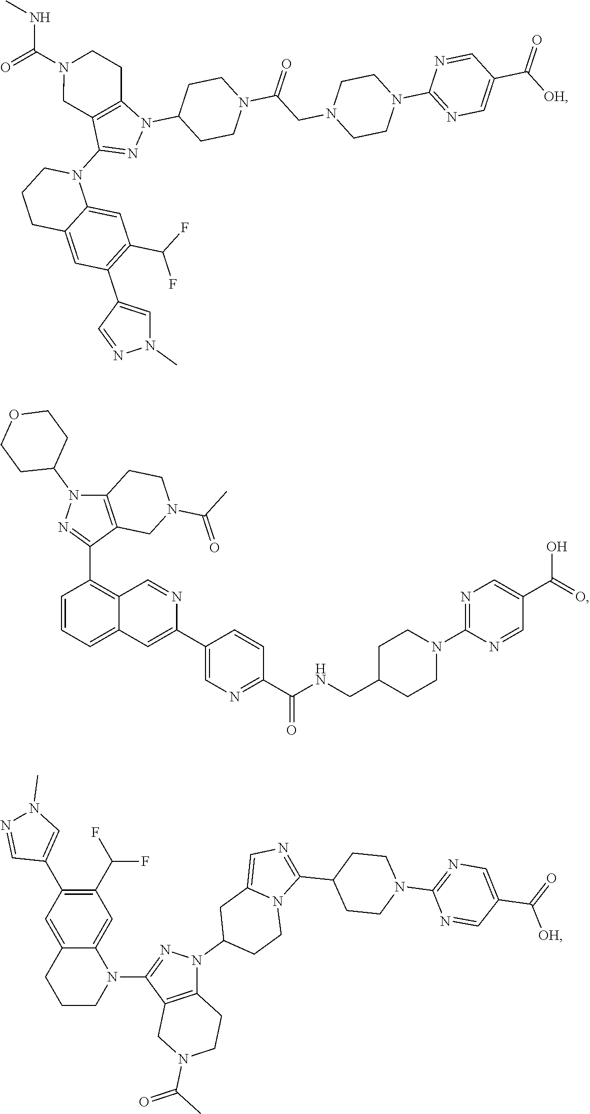 [SMILES: CNC(=O)N1CCc2c(c(N3CCCc4cc(-c5cnn(C)c5)c(C(F)F)cc43)nn2C2CCN(C(=O)CN3CCN(c4ncc(C(=O)O)cn4)CC3)CC2)C1 ; CC(=O)N1CCc2c(c(-c3cccc4cc(-c5ccc(C(=O)NCC6CCN(c7ncc(C(=O)O)cn7)CC6)nc5)ncc34)nn2C2CCOCC2)C1 ; CC(=O)N1CCc2c(c(N3CCCc4cc(-c5cnn(C)c5)c(C(F)F)cc43)nn2C2CCn3c(cnc3C3CCN(c4ncc(C(=O)O)cn4)CC3)C2)C1]

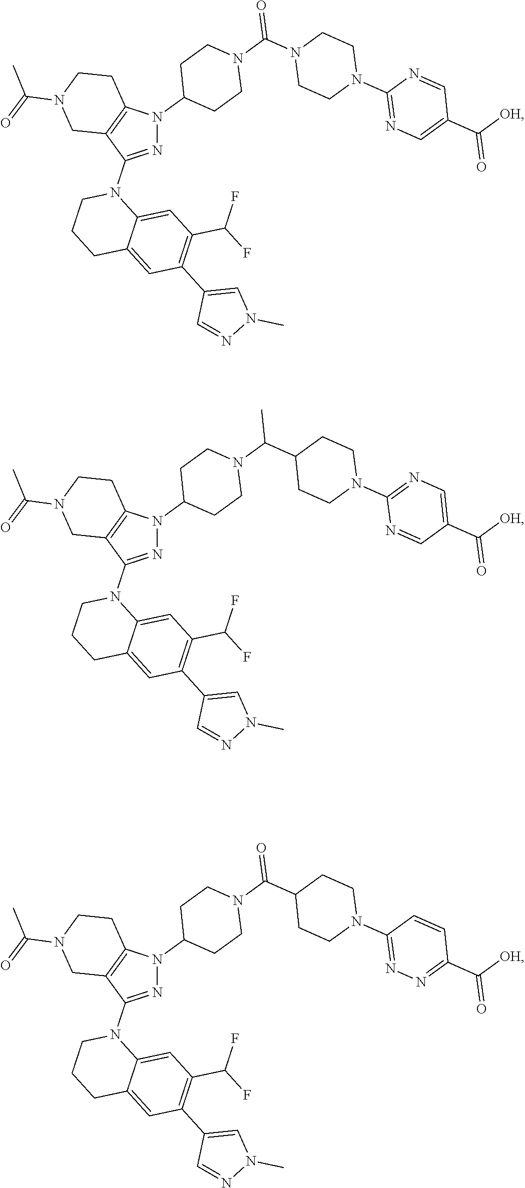 [SMILES: CC(=O)N1CCc2c(c(N3CCCc4cc(-c5cnn(C)c5)c(C(F)F)cc43)nn2C2CCN(C(=O)N3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(C(C)C3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(C(=O)C3CCN(c4ccc(C(=O)O)nn4)CC3)CC2)C1]

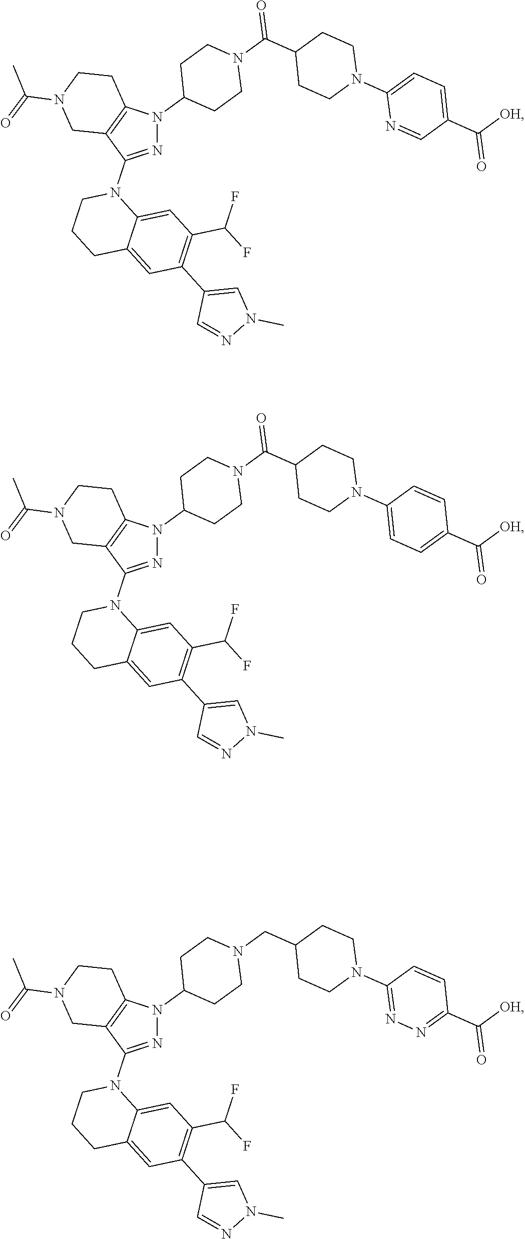 [SMILES: CC(=O)N1CCc2c(c(N3CCCc4cc(-c5cnn(C)c5)c(C(F)F)cc43)nn2C2CCN(C(=O)C3CCN(c4ccc(C(=O)O)cn4)CC3)CC2)C1 ; CC(=O)N1CCc2c(c(N3CCCc4cc(-c5cnn(C)c5)c(C(F)F)cc43)nn2C2CCN(C(=O)C3CCN(c4ccc(C(=O)O)cc4)CC3)CC2)C1 ; CC(=O)N1CCc2c(c(N3CCCc4cc(-c5cnn(C)c5)c(C(F)F)cc43)nn2C2CCN(CC3CCN(c4ccc(C(=O)O)nn4)CC3)CC2)C1]

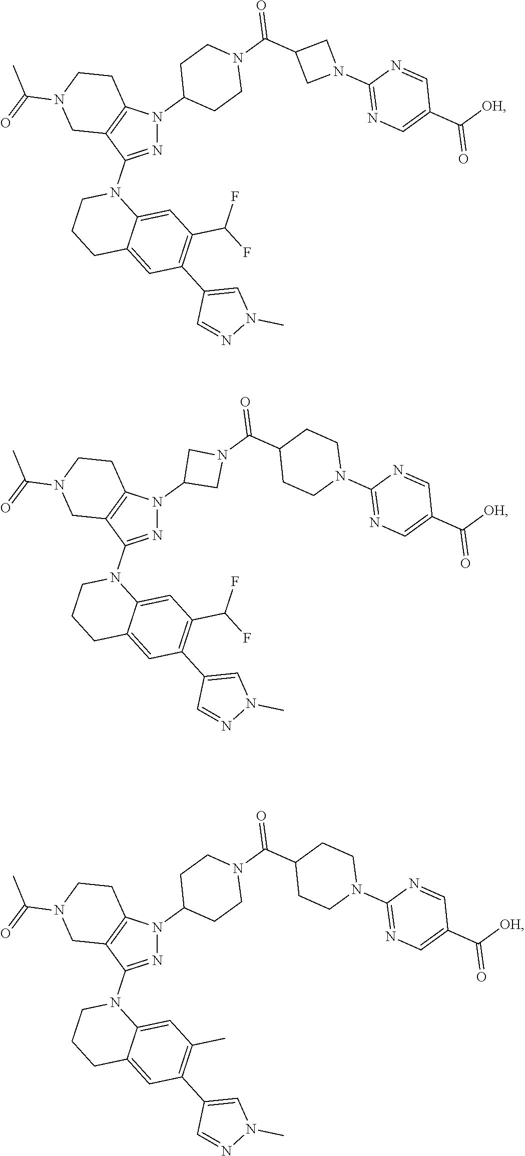 [SMILES: CC(=O)N1CCc2c(c(N3CCCc4cc(-c5cnn(C)c5)c(C(F)F)cc43)nn2C2CCN(C(=O)C3CN(c4ncc(C(=O)O)cn4)C3)CC2)C1 ; CC(=O)N1CCc2c(c(N3CCCc4cc(-c5cnn(C)c5)c(C(F)F)cc43)nn2C2CN(C(=O)C3CCN(c4ncc(C(=O)O)cn4)CC3)C2)C1 ; CC(=O)N1CCc2c(c(N3CCCc4cc(-c5cnn(C)c5)c(C)cc43)nn2C2CCN(C(=O)C3CCN(c4ncc(C(=O)O)cn4)CC3)CC2)C1]

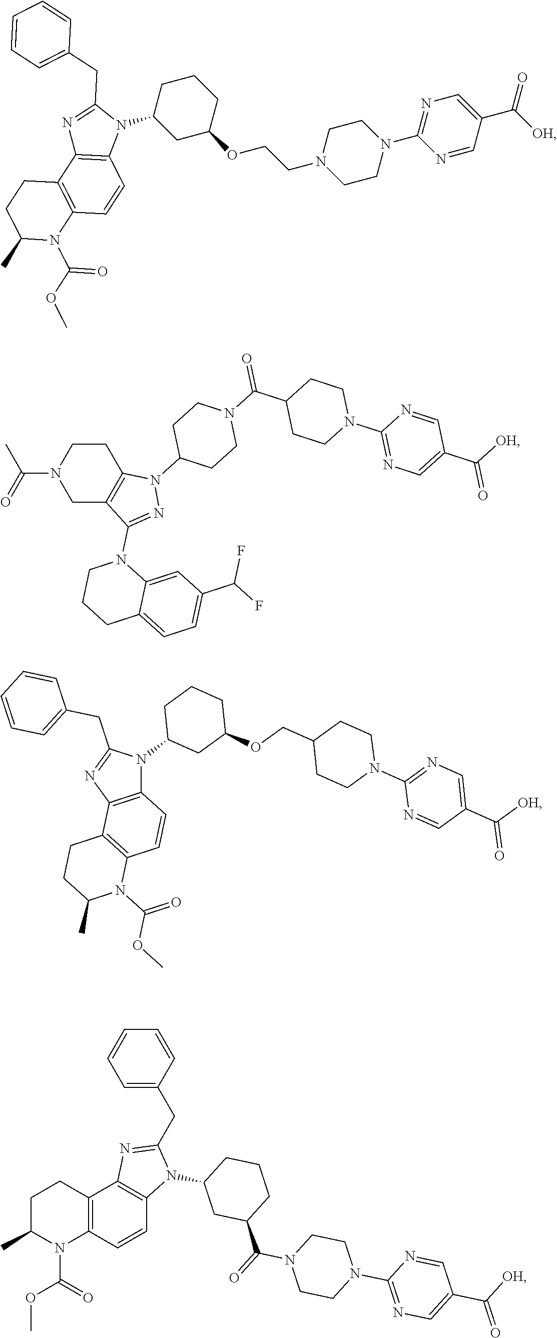 [SMILES: COC(=O)N1c2ccc3c(nc(Cc4ccccc4)n3[C@@H]3CCC[C@@H](OCCN4CCN(c5ncc(C(=O)O)cn5)CC4)C3)c2CC[C@@H]1C ; CC(=O)N1CCc2c(c(N3CCCc4ccc(C(F)F)cc43)nn2C2CCN(C(=O)C3CCN(c4ncc(C(=O)O)cn4)CC3)CC2)C1 ; COC(=O)N1c2ccc3c(nc(Cc4ccccc4)n3[C@@H]3CCC[C@@H](OCC4CCN(c5ncc(C(=O)O)cn5)CC4)C3)c2CC[C@@H]1C ; COC(=O)N1c2ccc3c(nc(Cc4ccccc4)n3[C@@H]3CCC[C@@H](C(=O)N4CCN(c5ncc(C(=O)O)cn5)CC4)C3)c2CC[C@@H]1C]

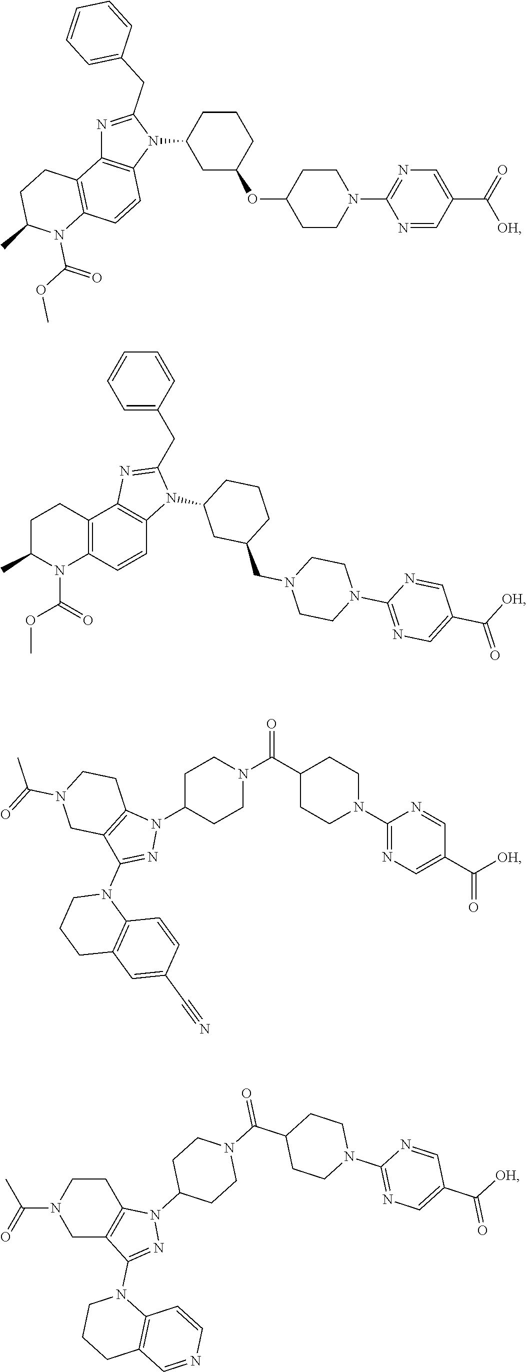 [SMILES: COC(=O)N1c2ccc3c(nc(Cc4ccccc4)n3[C@@H]3CCC[C@@H](OC4CCN(c5ncc(C(=O)O)cn5)CC4)C3)c2CC[C@@H]1C ; COC(=O)N1c2ccc3c(nc(Cc4ccccc4)n3[C@@H]3CCC[C@@H](CN4CCN(c5ncc(C(=O)O)cn5)CC4)C3)c2CC[C@@H]1C ; CC(=O)N1CCc2c(c(N3CCCc4cc(C#N)ccc43)nn2C2CCN(C(=O)C3CCN(c4ncc(C(=O)O)cn4)CC3)CC2)C1 ; CC(=O)N1CCc2c(c(N3CCCc4cnccc43)nn2C2CCN(C(=O)C3CCN(c4ncc(C(=O)O)cn4)CC3)CC2)C1]

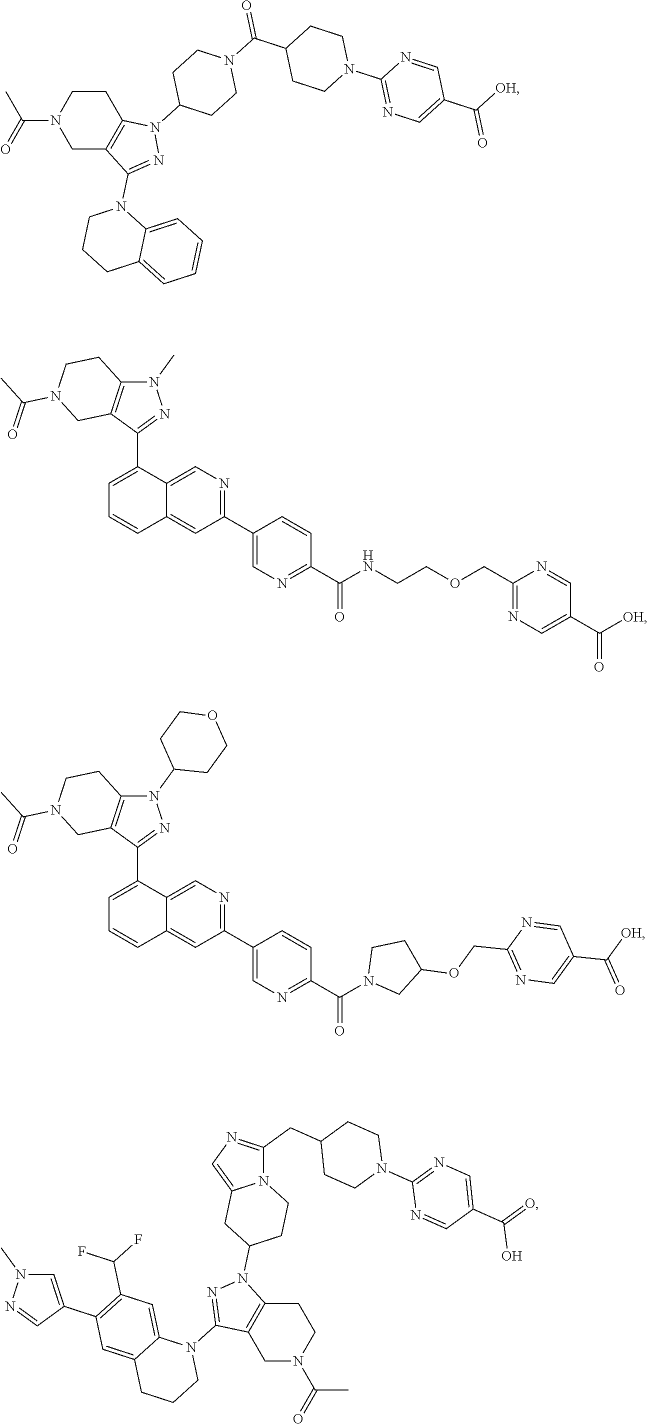 [SMILES: CC(=O)N1CCc2c(c(N3CCCc4ccccc43)nn2C2CCN(C(=O)C3CCN(c4ncc(C(=O)O)cn4)CC3)CC2)C1 ; CC(=O)N1CCc2c(c(-c3cccc4cc(-c5ccc(C(=O)NCCOCc6ncc(C(=O)O)cn6)nc5)ncc34)nn2C)C1 ; CC(=O)N1CCc2c(c(-c3cccc4cc(-c5ccc(C(=O)N6CCC(OCc7ncc(C(=O)O)cn7)C6)nc5)ncc34)nn2C2CCOCC2)C1 ; CC(=O)N1CCc2c(c(N3CCCc4cc(-c5cnn(C)c5)c(C(F)F)cc43)nn2C2CCn3c(cnc3CC3CCN(c4ncc(C(=O)O)cn4)CC3)C2)C1]

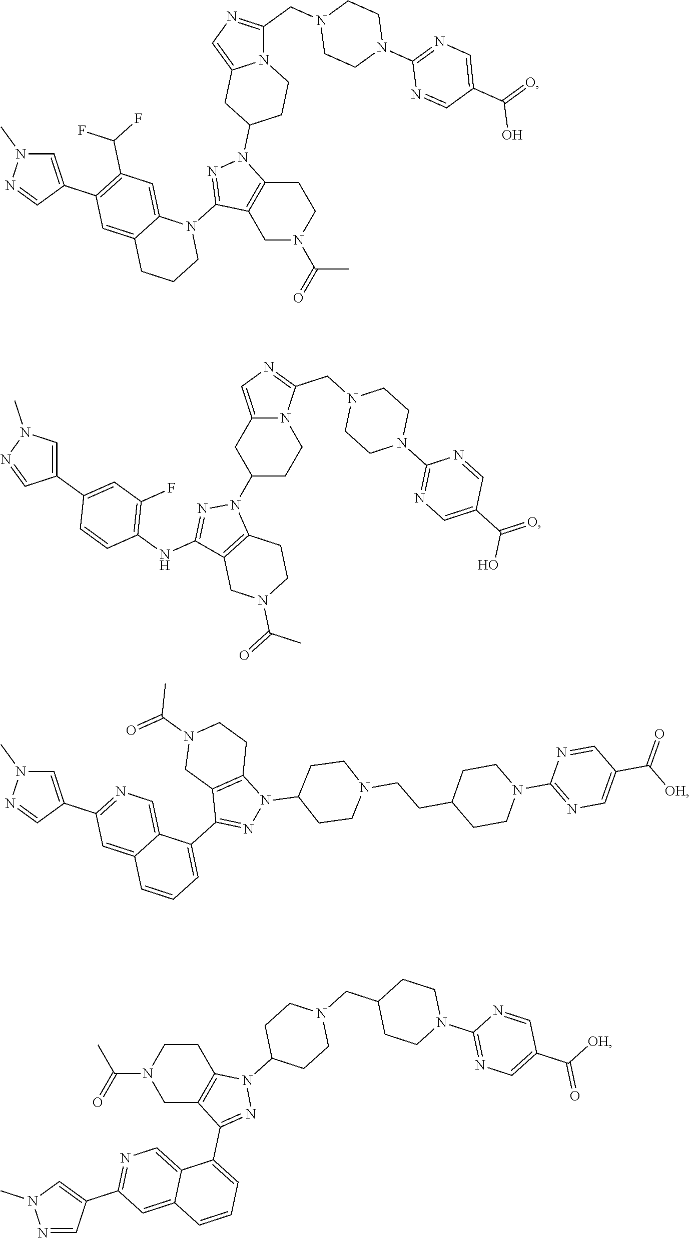 [SMILES: CC(=O)N1CCc2c(c(N3CCCc4cc(-c5cnn(C)c5)c(C(F)F)cc43)nn2C2CCn3c(cnc3CN3CCN(c4ncc(C(=O)O)cn4)CC3)C2)C1 ; CC(=O)N1CCc2c(c(Nc3ccc(-c4cnn(C)c4)cc3F)nn2C2CCn3c(cnc3CN3CCN(c4ncc(C(=O)O)cn4)CC3)C2)C1 ; CC(=O)N1CCc2c(c(-c3cccc4cc(-c5cnn(C)c5)ncc34)nn2C2CCN(CCC3CCN(c4ncc(C(=O)O)cn4)CC3)CC2)C1 ; CC(=O)N1CCc2c(c(-c3cccc4cc(-c5cnn(C)c5)ncc34)nn2C2CCN(CC3CCN(c4ncc(C(=O)O)cn4)CC3)CC2)C1]

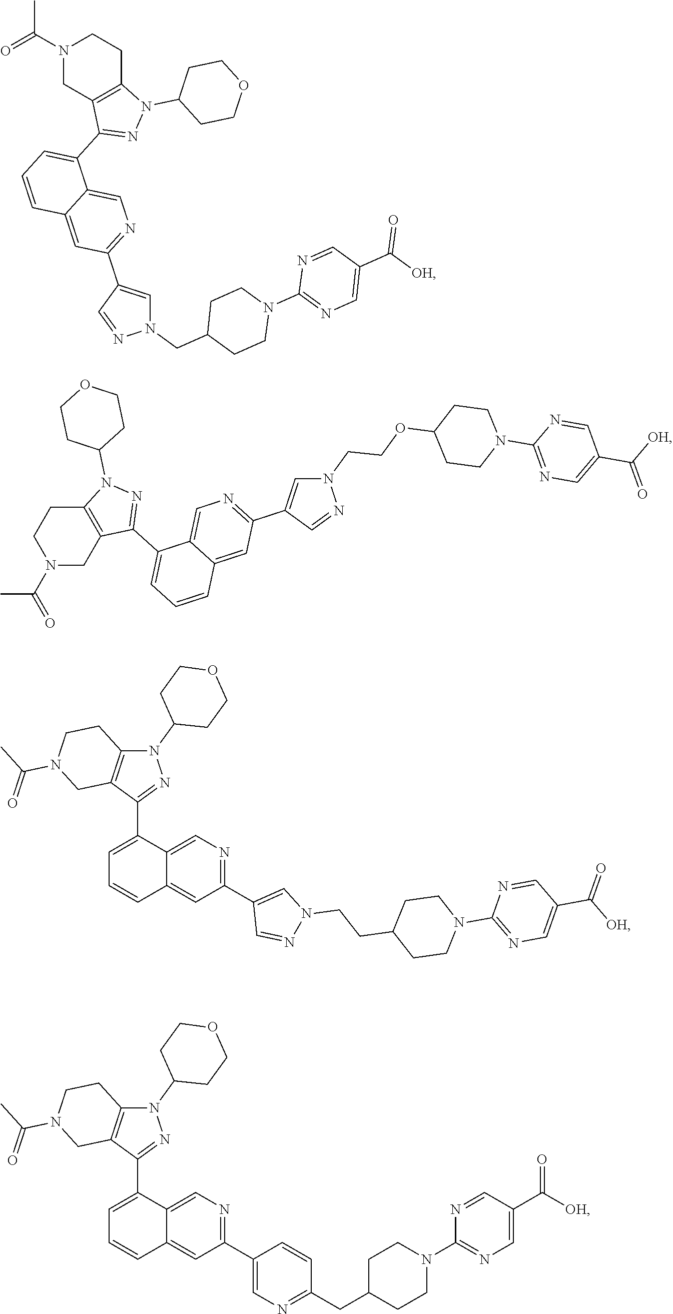 [SMILES: CC(=O)N1CCc2c(c(-c3cccc4cc(-c5cnn(CC6CCN(c7ncc(C(=O)O)cn7)CC6)c5)ncc34)nn2C2CCOCC2)C1 ; CC(=O)N1CCc2c(c(-c3cccc4cc(-c5cnn(CCOC6CCN(c7ncc(C(=O)O)cn7)CC6)c5)ncc34)nn2C2CCOCC2)C1 ; CC(=O)N1CCc2c(c(-c3cccc4cc(-c5cnn(CCC6CCN(c7ncc(C(=O)O)cn7)CC6)c5)ncc34)nn2C2CCOCC2)C1 ; CC(=O)N1CCc2c(c(-c3cccc4cc(-c5ccc(CC6CCN(c7ncc(C(=O)O)cn7)CC6)nc5)ncc34)nn2C2CCOCC2)C1]

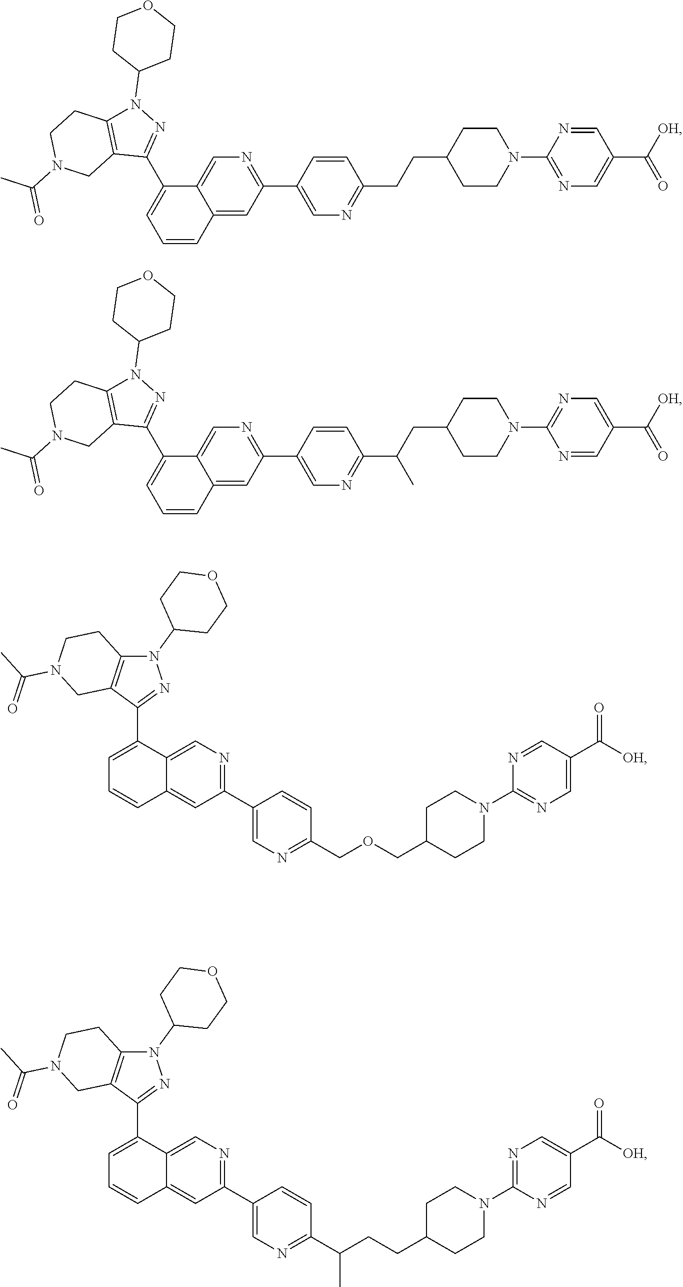

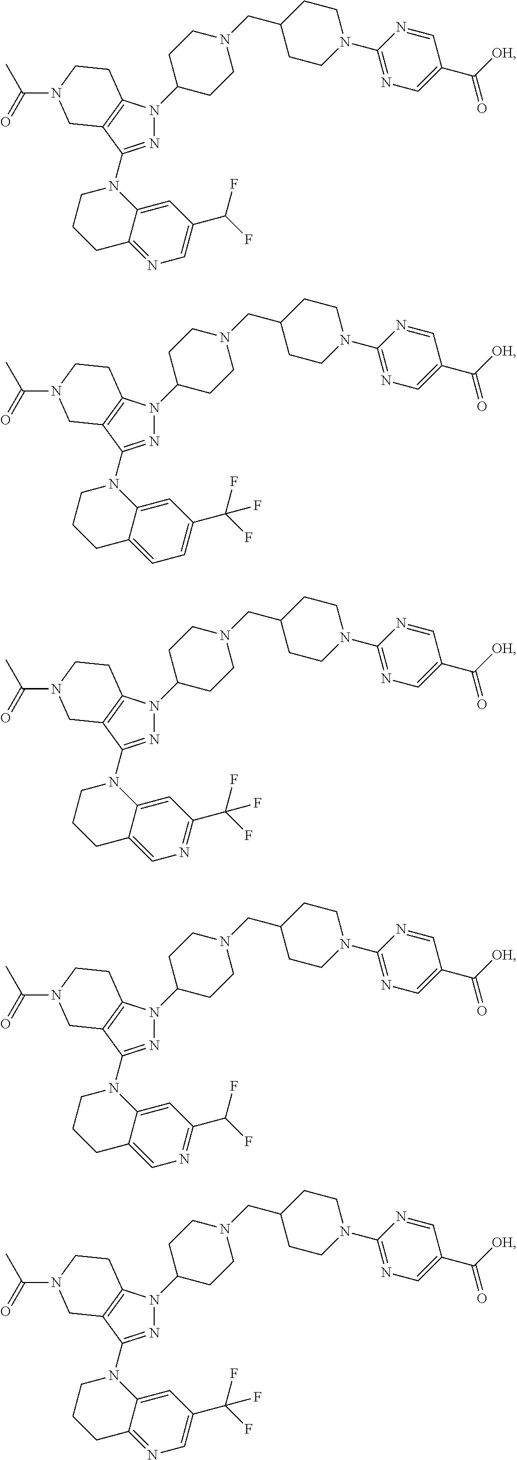 [SMILES: CC(=O)N1CCc2c(c(N3CCCc4ncc(C(F)F)cc43)nn2C2CCN(CC3CCN(c4ncc(C(=O)O)cn4)CC3)CC2)C1 ; CC(=O)N1CCc2c(c(N3CCCc4ccc(C(F)(F)F)cc43)nn2C2CCN(CC3CCN(c4ncc(C(=O)O)cn4)CC3)CC2)C1 ; CC(=O)N1CCc2c(c(N3CCCc4cnc(C(F)(F)F)cc43)nn2C2CCN(CC3CCN(c4ncc(C(=O)O)cn4)CC3)CC2)C1 ; CC(=O)N1CCc2c(c(N3CCCc4cnc(C(F)F)cc43)nn2C2CCN(CC3CCN(c4ncc(C(=O)O)cn4)CC3)CC2)C1 ; CC(=O)N1CCc2c(c(N3CCCc4ncc(C(F)(F)F)cc43)nn2C2CCN(CC3CCN(c4ncc(C(=O)O)cn4)CC3)CC2)C1]

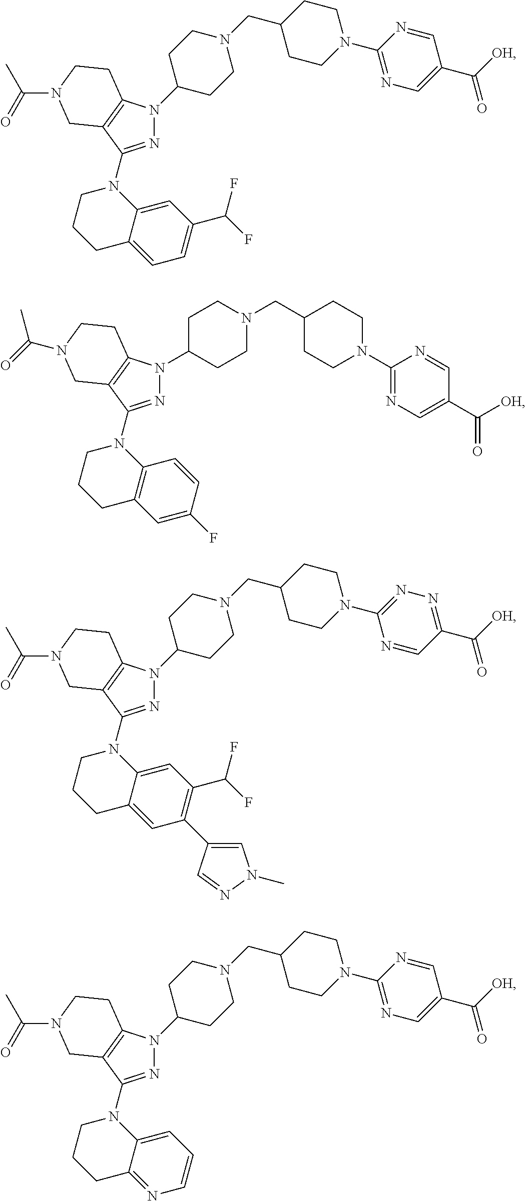 [SMILES: CC(=O)N1CCc2c(c(N3CCCc4ccc(C(F)F)cc43)nn2C2CCN(CC3CCN(c4ncc(C(=O)O)cn4)CC3)CC2)C1 ; CC(=O)N1CCc2c(c(N3CCCc4cc(F)ccc43)nn2C2CCN(CC3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(CC3CCN(c4ncc(C(=O)O)nn4)CC3)CC2)C1 ; CC(=O)N1CCc2c(c(N3CCCc4ncccc43)nn2C2CCN(CC3CCN(c4ncc(C(=O)O)cn4)CC3)CC2)C1]

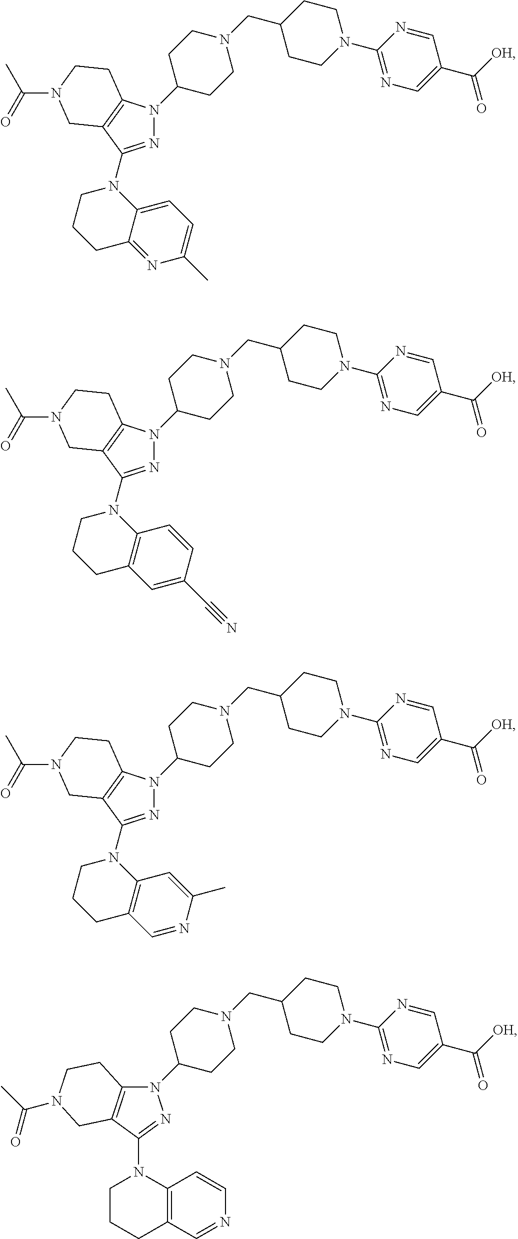 [SMILES: CC(=O)N1CCc2c(c(N3CCCc4nc(C)ccc43)nn2C2CCN(CC3CCN(c4ncc(C(=O)O)cn4)CC3)CC2)C1 ; CC(=O)N1CCc2c(c(N3CCCc4cc(C#N)ccc43)nn2C2CCN(CC3CCN(c4ncc(C(=O)O)cn4)CC3)CC2)C1 ; CC(=O)N1CCc2c(c(N3CCCc4cnc(C)cc43)nn2C2CCN(CC3CCN(c4ncc(C(=O)O)cn4)CC3)CC2)C1 ; CC(=O)N1CCc2c(c(N3CCCc4cnccc43)nn2C2CCN(CC3CCN(c4ncc(C(=O)O)cn4)CC3)CC2)C1]

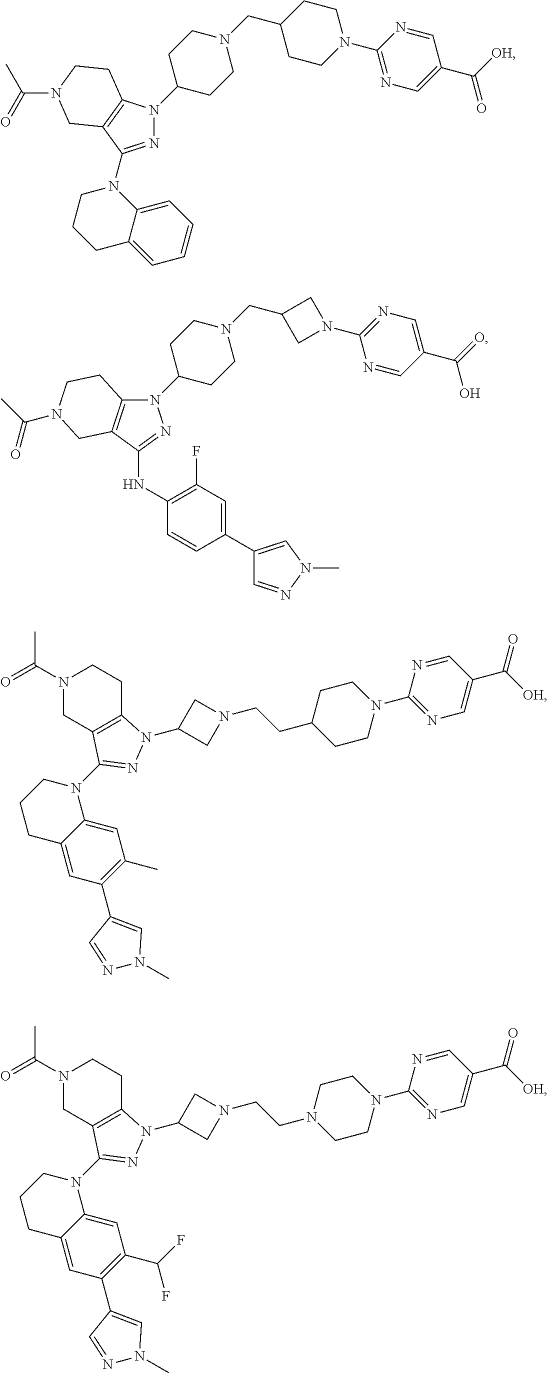 [SMILES: CC(=O)N1CCc2c(c(N3CCCc4ccccc43)nn2C2CCN(CC3CCN(c4ncc(C(=O)O)cn4)CC3)CC2)C1 ; CC(=O)N1CCc2c(c(Nc3ccc(-c4cnn(C)c4)cc3F)nn2C2CCN(CC3CN(c4ncc(C(=O)O)cn4)C3)CC2)C1 ; CC(=O)N1CCc2c(c(N3CCCc4cc(-c5cnn(C)c5)c(C)cc43)nn2C2CN(CCC3CCN(c4ncc(C(=O)O)cn4)CC3)C2)C1 ; CC(=O)N1CCc2c(c(N3CCCc4cc(-c5cnn(C)c5)c(C(F)F)cc43)nn2C2CN(CCN3CCN(c4ncc(C(=O)O)cn4)CC3)C2)C1]

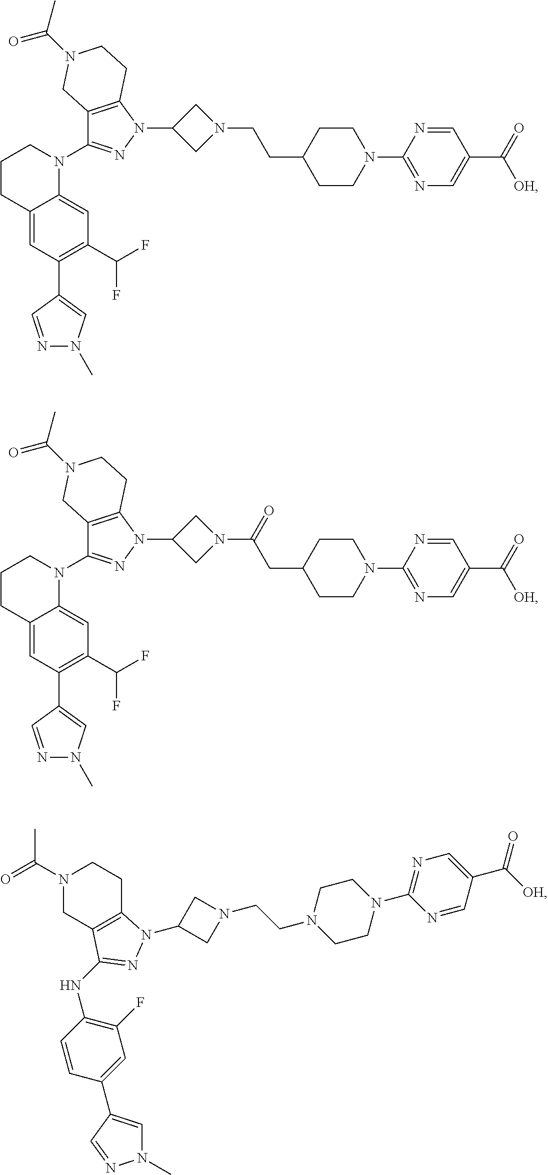 [SMILES: CC(=O)N1CCc2c(c(N3CCCc4cc(-c5cnn(C)c5)c(C(F)F)cc43)nn2C2CN(CCC3CCN(c4ncc(C(=O)O)cn4)CC3)C2)C1 ; CC(=O)N1CCc2c(c(N3CCCc4cc(-c5cnn(C)c5)c(C(F)F)cc43)nn2C2CN(C(=O)CC3CCN(c4ncc(C(=O)O)cn4)CC3)C2)C1 ; CC(=O)N1CCc2c(c(Nc3ccc(-c4cnn(C)c4)cc3F)nn2C2CN(CCN3CCN(c4ncc(C(=O)O)cn4)CC3)C2)C1]

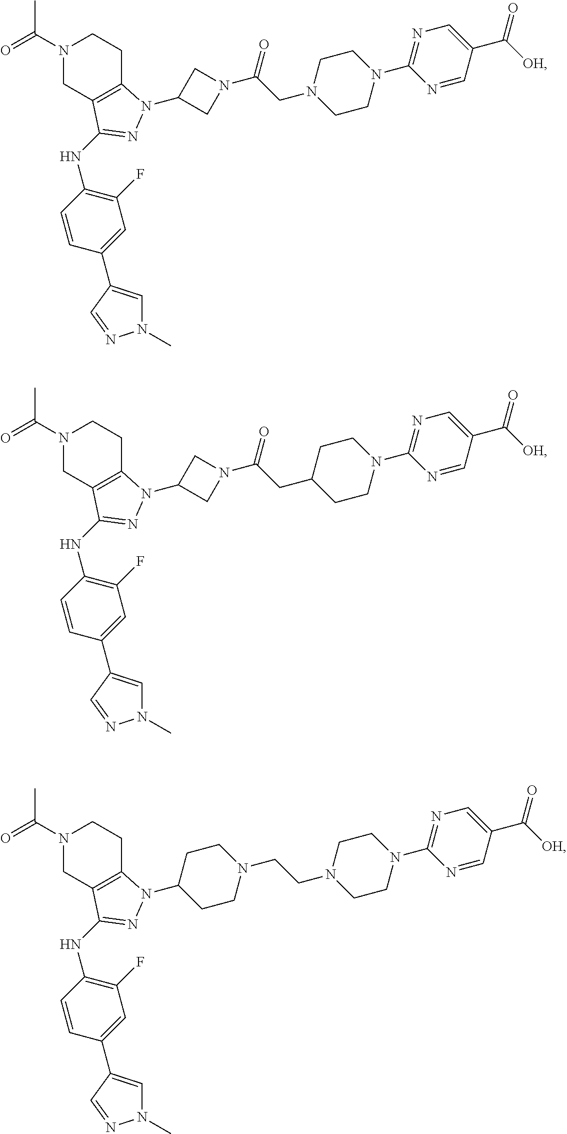 [SMILES: CC(=O)N1CCc2c(c(Nc3ccc(-c4cnn(C)c4)cc3F)nn2C2CN(C(=O)CN3CCN(c4ncc(C(=O)O)cn4)CC3)C2)C1 ; CC(=O)N1CCc2c(c(Nc3ccc(-c4cnn(C)c4)cc3F)nn2C2CN(C(=O)CC3CCN(c4ncc(C(=O)O)cn4)CC3)C2)C1 ; CC(=O)N1CCc2c(c(Nc3ccc(-c4cnn(C)c4)cc3F)nn2C2CCN(CCN3CCN(c4ncc(C(=O)O)cn4)CC3)CC2)C1]

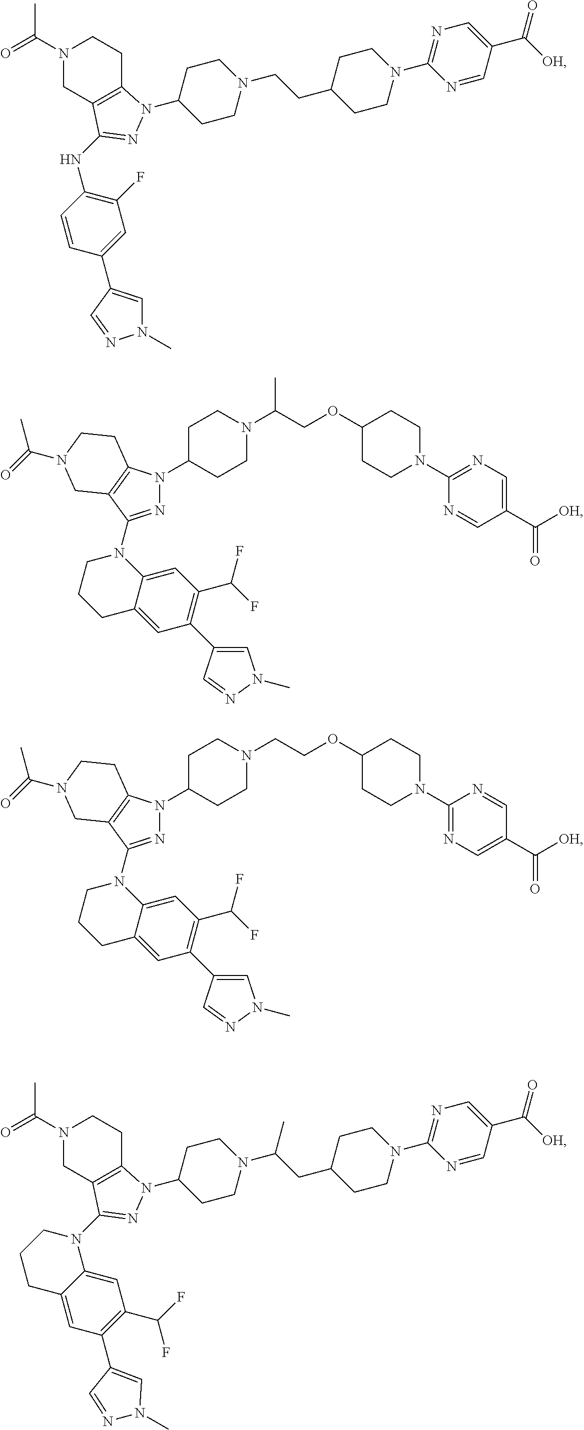

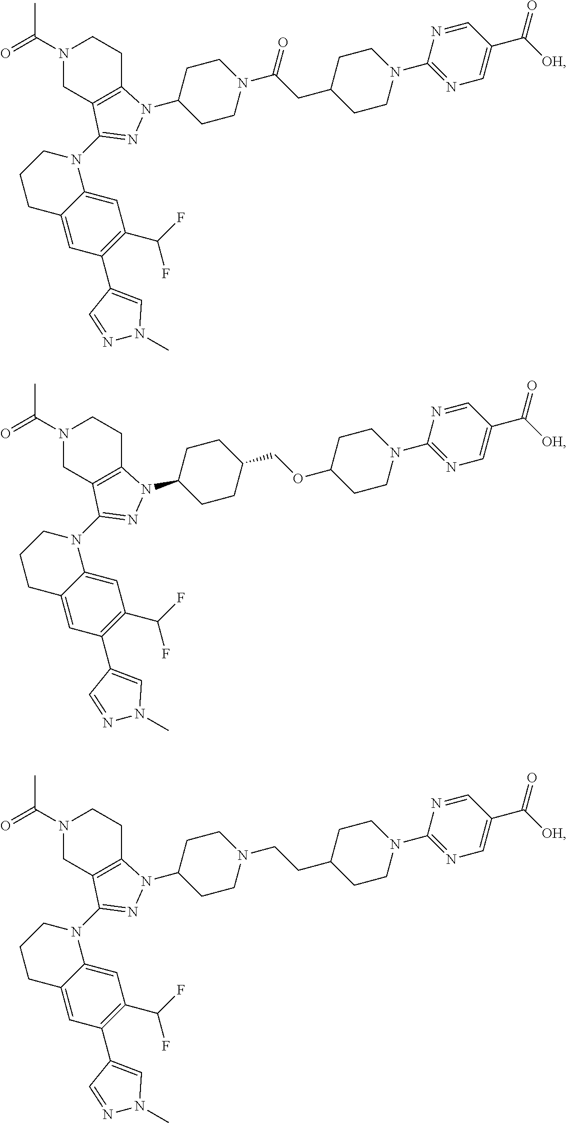 [SMILES: CC(=O)N1CCc2c(c(N3CCCc4cc(-c5cnn(C)c5)c(C(F)F)cc43)nn2C2CCN(C(=O)CC3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@H](COC3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(CCC3CCN(c4ncc(C(=O)O)cn4)CC3)CC2)C1]

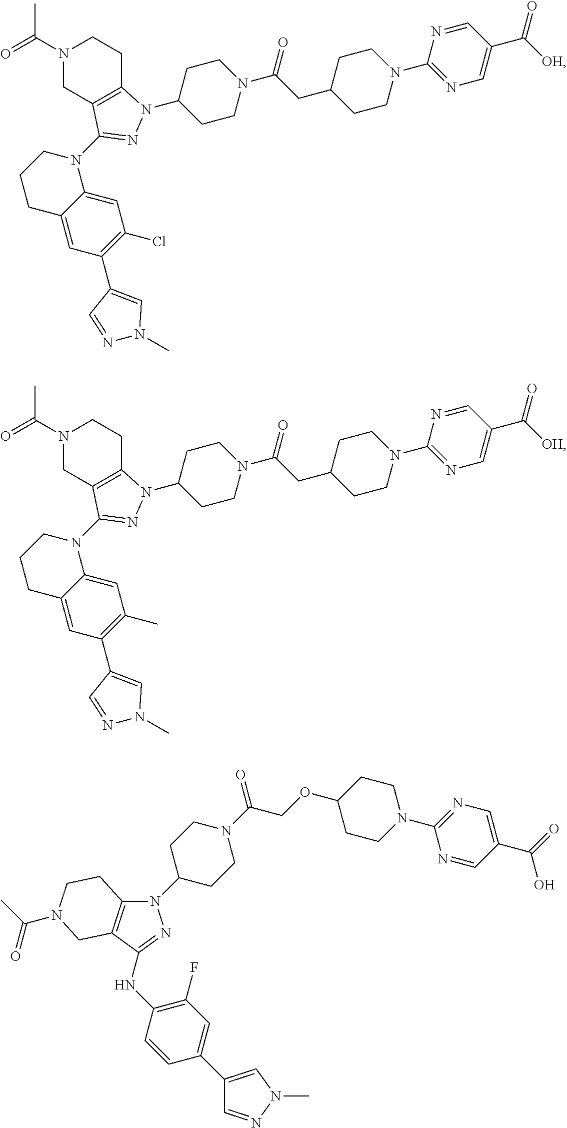 [SMILES: CC(=O)N1CCc2c(c(N3CCCc4cc(-c5cnn(C)c5)c(Cl)cc43)nn2C2CCN(C(=O)CC3CCN(c4ncc(C(=O)O)cn4)CC3)CC2)C1 ; CC(=O)N1CCc2c(c(N3CCCc4cc(-c5cnn(C)c5)c(C)cc43)nn2C2CCN(C(=O)CC3CCN(c4ncc(C(=O)O)cn4)CC3)CC2)C1 ; CC(=O)N1CCc2c(c(Nc3ccc(-c4cnn(C)c4)cc3F)nn2C2CCN(C(=O)COC3CCN(c4ncc(C(=O)O)cn4)CC3)CC2)C1]

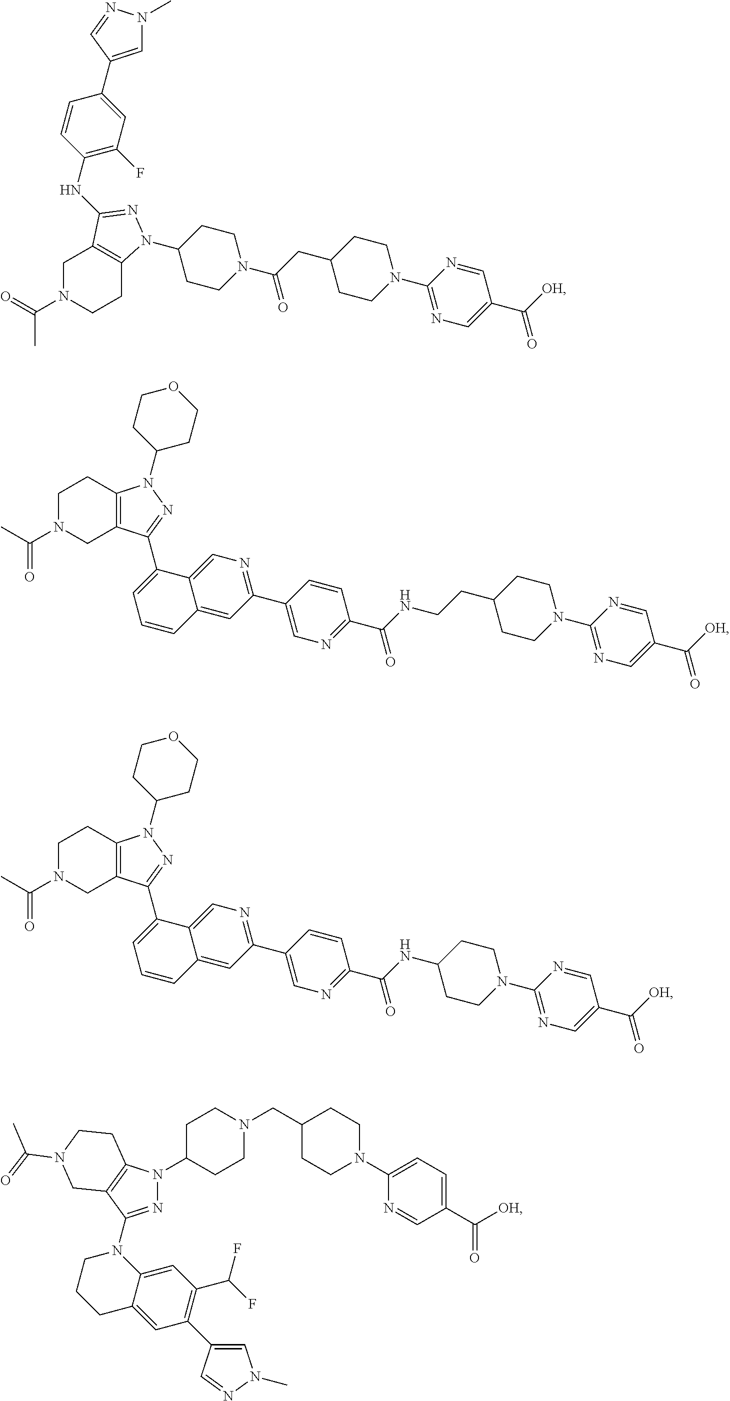 [SMILES: CC(=O)N1CCc2c(c(Nc3ccc(-c4cnn(C)c4)cc3F)nn2C2CCN(C(=O)CC3CCN(c4ncc(C(=O)O)cn4)CC3)CC2)C1 ; CC(=O)N1CCc2c(c(-c3cccc4cc(-c5ccc(C(=O)NCCC6CCN(c7ncc(C(=O)O)cn7)CC6)nc5)ncc34)nn2C2CCOCC2)C1 ; CC(=O)N1CCc2c(c(-c3cccc4cc(-c5ccc(C(=O)NC6CCN(c7ncc(C(=O)O)cn7)CC6)nc5)ncc34)nn2C2CCOCC2)C1 ; CC(=O)N1CCc2c(c(N3CCCc4cc(-c5cnn(C)c5)c(C(F)F)cc43)nn2C2CCN(CC3CCN(c4ccc(C(=O)O)cn4)CC3)CC2)C1]

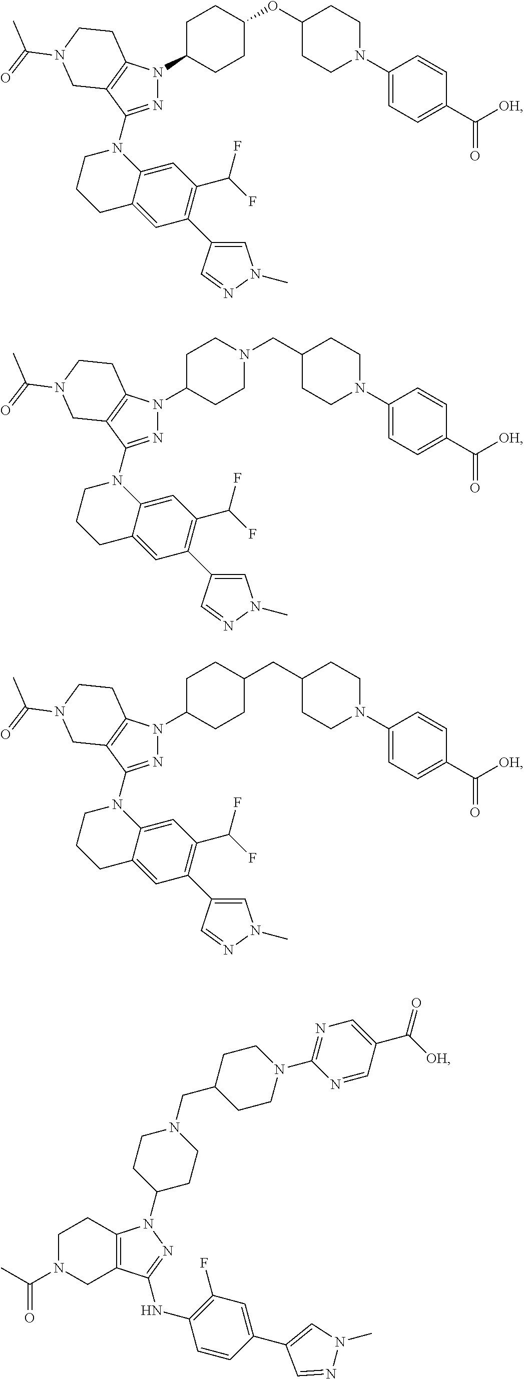

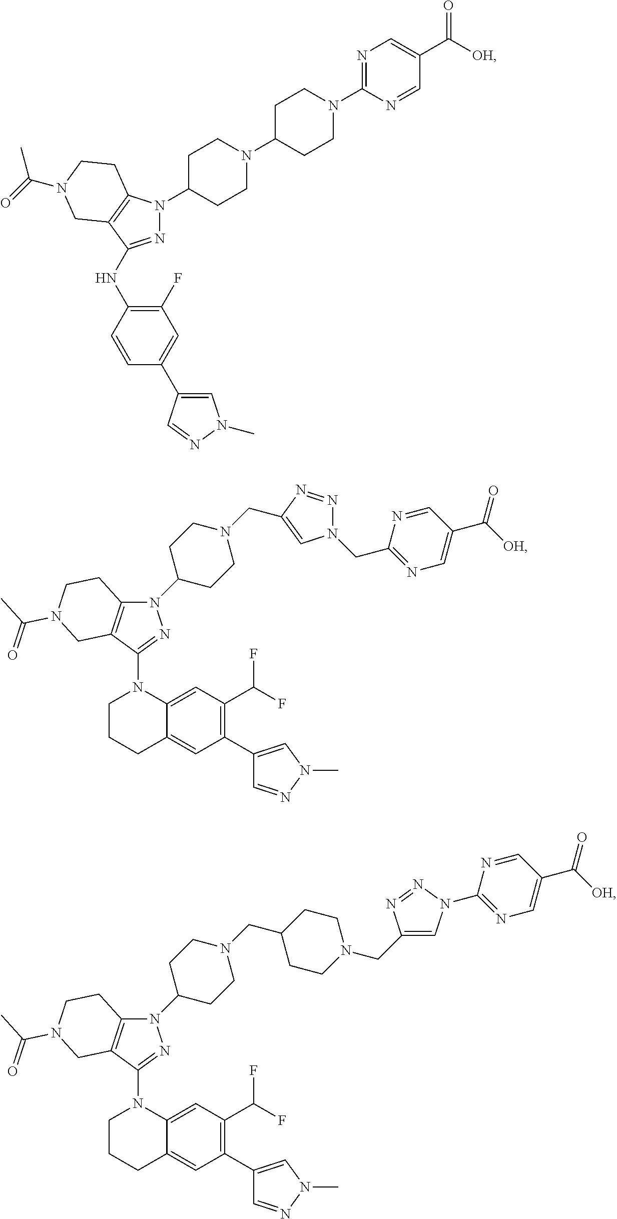 [SMILES: CC(=O)N1CCc2c(c(Nc3ccc(-c4cnn(C)c4)cc3F)nn2C2CCN(C3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(Cc3cn(Cc4ncc(C(=O)O)cn4)nn3)CC2)C1 ; CC(=O)N1CCc2c(c(N3CCCc4cc(-c5cnn(C)c5)c(C(F)F)cc43)nn2C2CCN(CC3CCN(Cc4cn(-c5ncc(C(=O)O)cn5)nn4)CC3)CC2)C1]

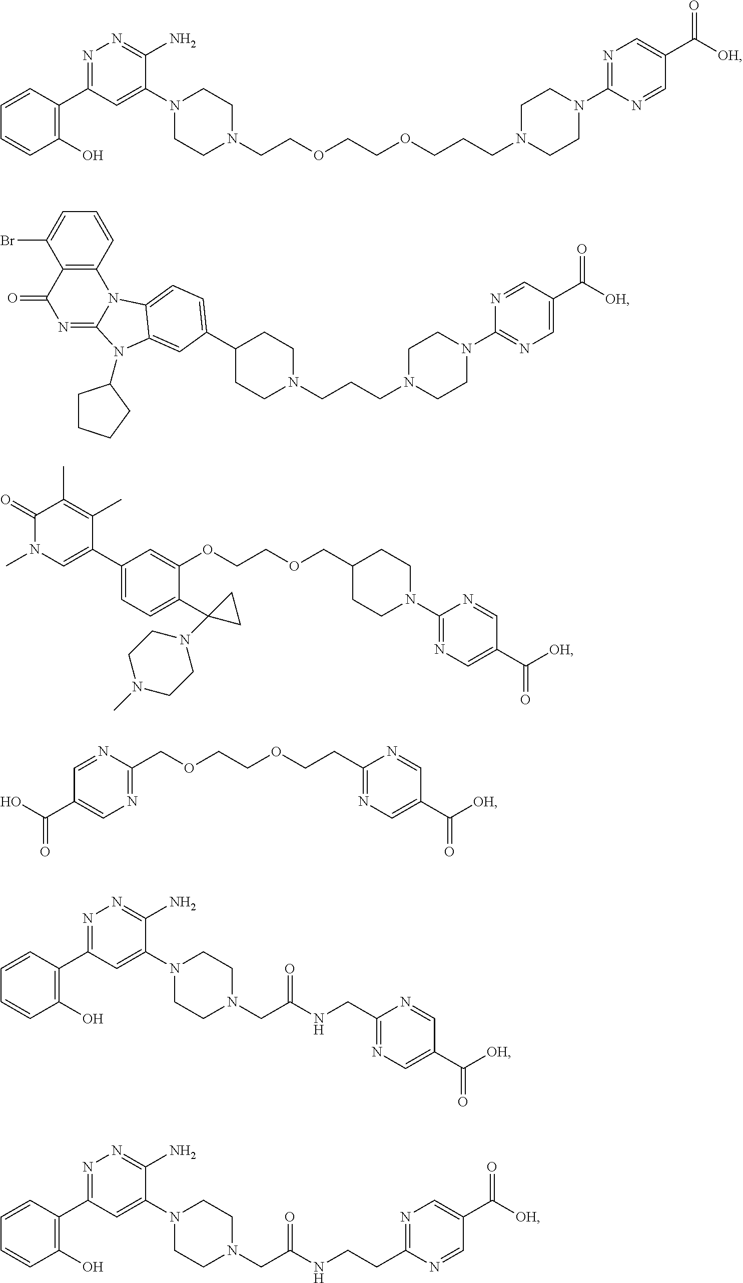

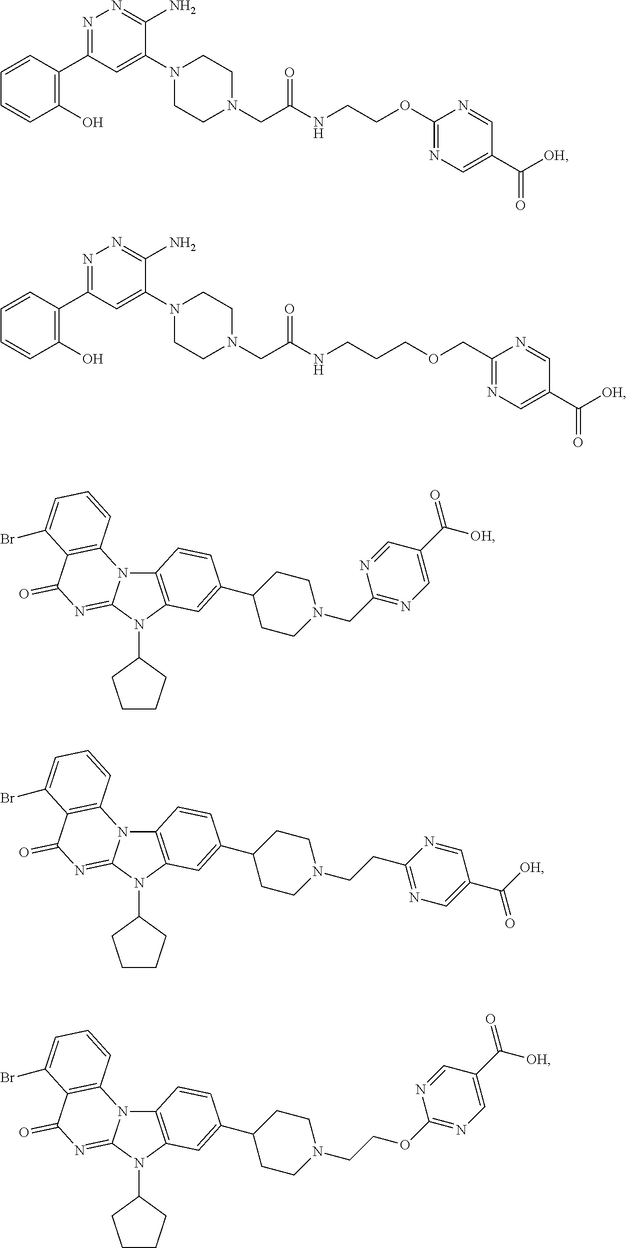 [SMILES: Nc1nnc(-c2ccccc2O)cc1N1CCN(CC(=O)NCCOc2ncc(C(=O)O)cn2)CC1 ; Nc1nnc(-c2ccccc2O)cc1N1CCN(CC(=O)NCCCOCc2ncc(C(=O)O)cn2)CC1 ; O=C(O)c1cnc(CN2CCC(c3ccc4c(c3)n(C3CCCC3)c3nc(=O)c5c(Br)cccc5n43)CC2)nc1 ; O=C(O)c1cnc(CCN2CCC(c3ccc4c(c3)n(C3CCCC3)c3nc(=O)c5c(Br)cccc5n43)CC2)nc1 ; O=C(O)c1cnc(OCCN2CCC(c3ccc4c(c3)n(C3CCCC3)c3nc(=O)c5c(Br)cccc5n43)CC2)nc1]

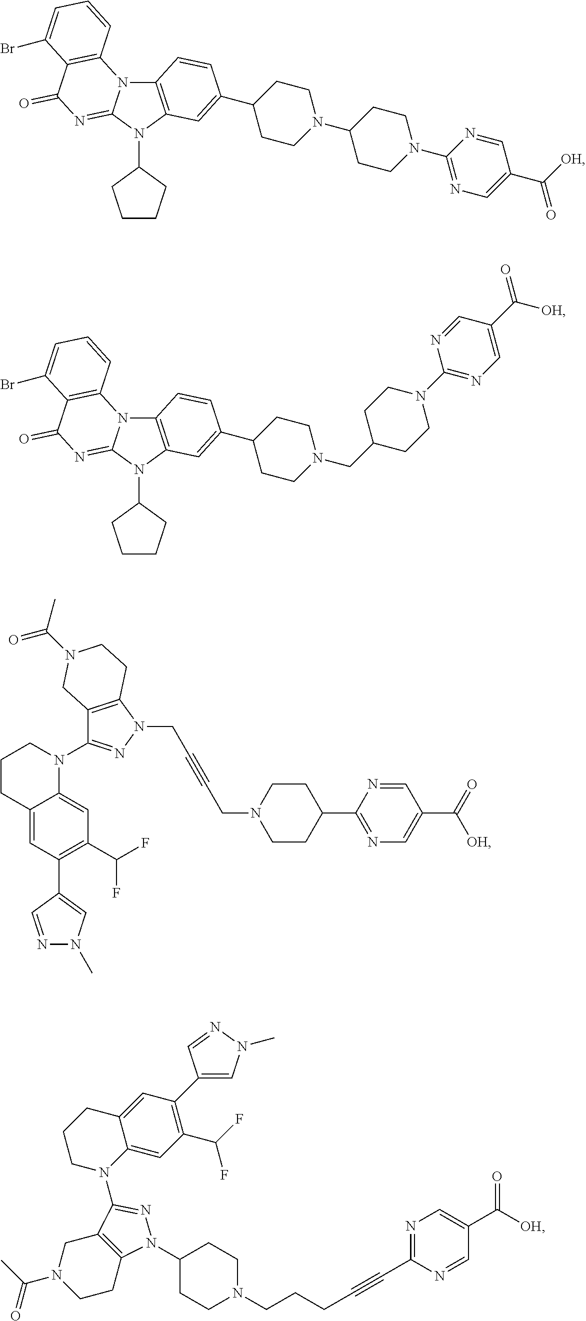 [SMILES: O=C(O)c1cnc(N2CCC(N3CCC(c4ccc5c(c4)n(C4CCCC4)c4nc(=O)c6c(Br)cccc6n54)CC3)CC2)nc1 ; O=C(O)c1cnc(N2CCC(CN3CCC(c4ccc5c(c4)n(C4CCCC4)c4nc(=O)c6c(Br)cccc6n54)CC3)CC2)nc1 ; CC(=O)N1CCc2c(c(N3CCCc4cc(-c5cnn(C)c5)c(C(F)F)cc43)nn2CC#CCN2CCC(c3ncc(C(=O)O)cn3)CC2)C1 ; CC(=O)N1CCc2c(c(N3CCCc4cc(-c5cnn(C)c5)c(C(F)F)cc43)nn2C2CCN(CCCC#Cc3ncc(C(=O)O)cn3)CC2)C1]

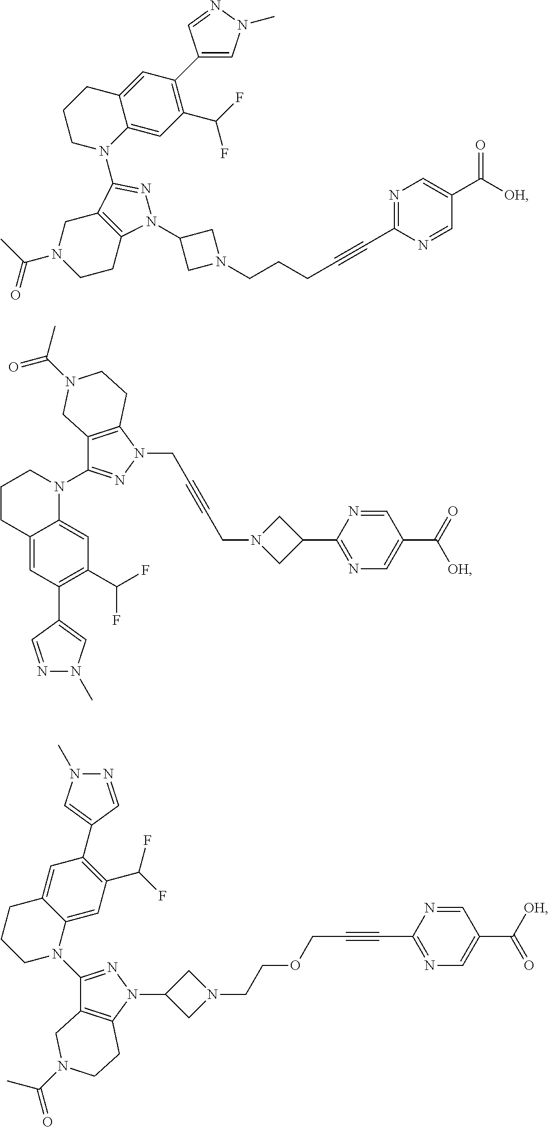 [SMILES: CC(=O)N1CCc2c(c(N3CCCc4cc(-c5cnn(C)c5)c(C(F)F)cc43)nn2C2CN(CCCC#Cc3ncc(C(=O)O)cn3)C2)C1 ; CC(=O)N1CCc2c(c(N3CCCc4cc(-c5cnn(C)c5)c(C(F)F)cc43)nn2CC#CCN2CC(c3ncc(C(=O)O)cn3)C2)C1 ; CC(=O)N1CCc2c(c(N3CCCc4cc(-c5cnn(C)c5)c(C(F)F)cc43)nn2C2CN(CCOCC#Cc3ncc(C(=O)O)cn3)C2)C1]

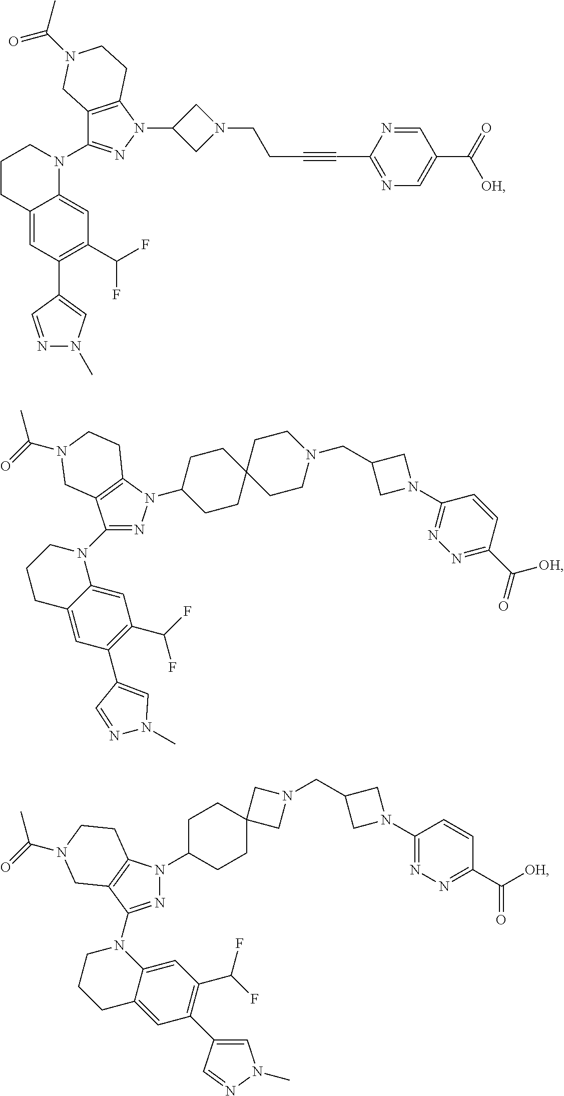 [SMILES: CC(=O)N1CCc2c(c(N3CCCc4cc(-c5cnn(C)c5)c(C(F)F)cc43)nn2C2CN(CCC#Cc3ncc(C(=O)O)cn3)C2)C1 ; CC(=O)N1CCc2c(c(N3CCCc4cc(-c5cnn(C)c5)c(C(F)F)cc43)nn2C2CCC3(CC2)CCN(CC2CN(c4ccc(C(=O)O)nn4)C2)CC3)C1 ; CC(=O)N1CCc2c(c(N3CCCc4cc(-c5cnn(C)c5)c(C(F)F)cc43)nn2C2CCC3(CC2)CN(CC2CN(c4ccc(C(=O)O)nn4)C2)C3)C1]

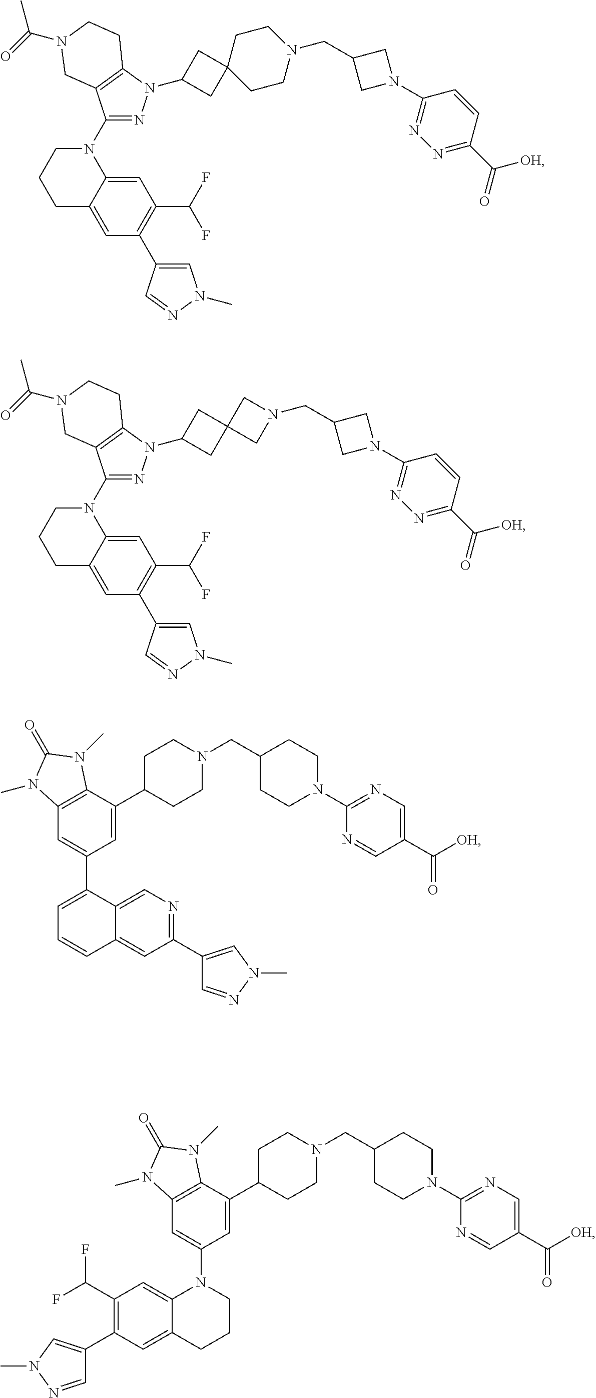 [SMILES: CC(=O)N1CCc2c(c(N3CCCc4cc(-c5cnn(C)c5)c(C(F)F)cc43)nn2C2CC3(CCN(CC4CN(c5ccc(C(=O)O)nn5)C4)CC3)C2)C1 ; CC(=O)N1CCc2c(c(N3CCCc4cc(-c5cnn(C)c5)c(C(F)F)cc43)nn2C2CC3(C2)CN(CC2CN(c4ccc(C(=O)O)nn4)C2)C3)C1 ; Cn1cc(-c2cc3cccc(-c4cc(C5CCN(CC6CCN(c7ncc(C(=O)O)cn7)CC6)CC5)c5c(c4)n(C)c(=O)n5C)c3cn2)cn1 ; Cn1cc(-c2cc3c(cc2C(F)F)N(c2cc(C4CCN(CC5CCN(c6ncc(C(=O)O)cn6)CC5)CC4)c4c(c2)n(C)c(=O)n4C)CCC3)cn1]

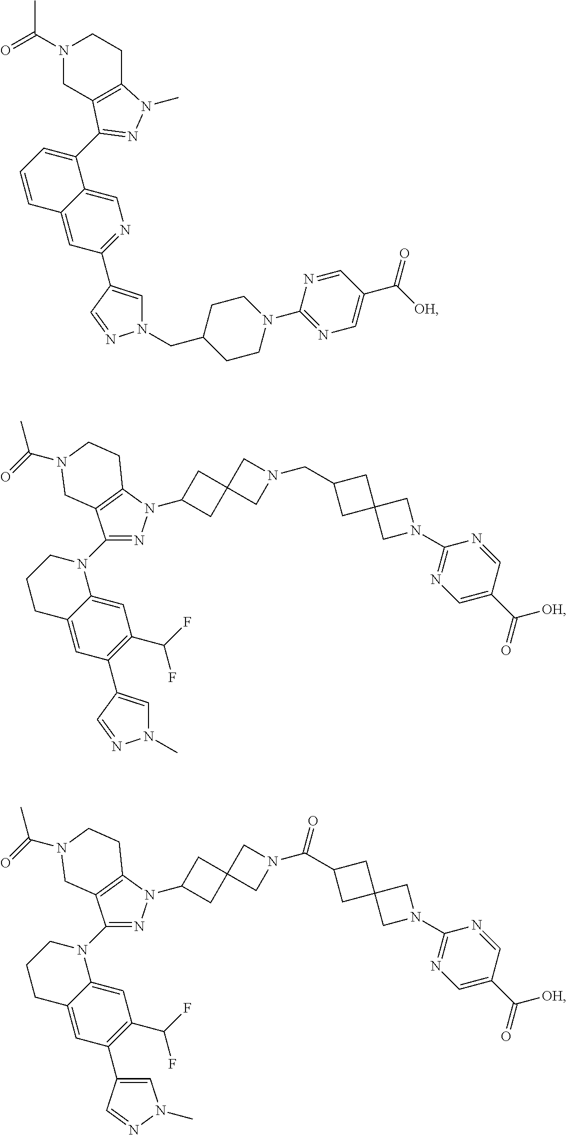 [SMILES: CC(=O)N1CCc2c(c(-c3cccc4cc(-c5cnn(CC6CCN(c7ncc(C(=O)O)cn7)CC6)c5)ncc34)nn2C)C1 ; CC(=O)N1CCc2c(c(N3CCCc4cc(-c5cnn(C)c5)c(C(F)F)cc43)nn2C2CC3(C2)CN(CC2CC4(C2)CN(c2ncc(C(=O)O)cn2)C4)C3)C1 ; CC(=O)N1CCc2c(c(N3CCCc4cc(-c5cnn(C)c5)c(C(F)F)cc43)nn2C2CC3(C2)CN(C(=O)C2CC4(C2)CN(c2ncc(C(=O)O)cn2)C4)C3)C1]

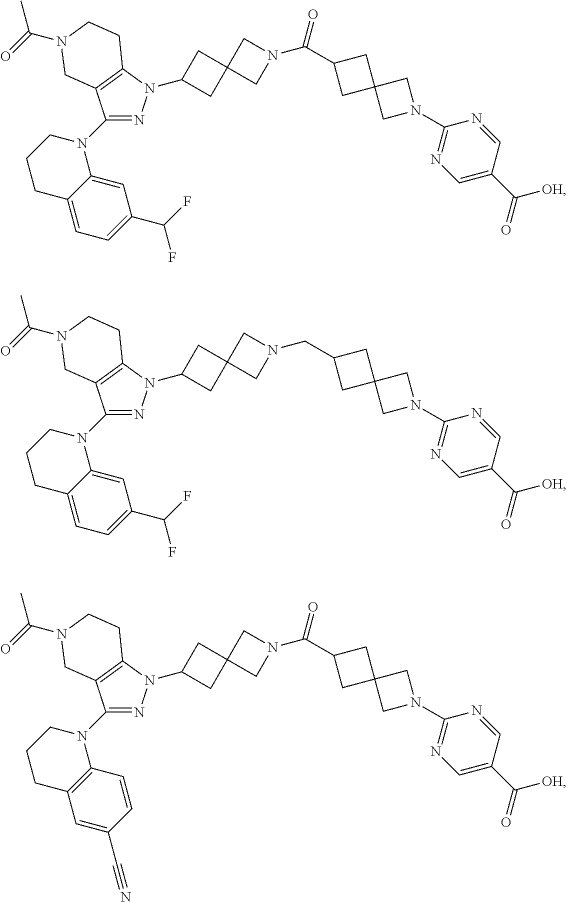 [SMILES: CC(=O)N1CCc2c(c(N3CCCc4ccc(C(F)F)cc43)nn2C2CC3(C2)CN(C(=O)C2CC4(C2)CN(c2ncc(C(=O)O)cn2)C4)C3)C1 ; CC(=O)N1CCc2c(c(N3CCCc4ccc(C(F)F)cc43)nn2C2CC3(C2)CN(CC2CC4(C2)CN(c2ncc(C(=O)O)cn2)C4)C3)C1 ; CC(=O)N1CCc2c(c(N3CCCc4cc(C#N)ccc43)nn2C2CC3(C2)CN(C(=O)C2CC4(C2)CN(c2ncc(C(=O)O)cn2)C4)C3)C1]

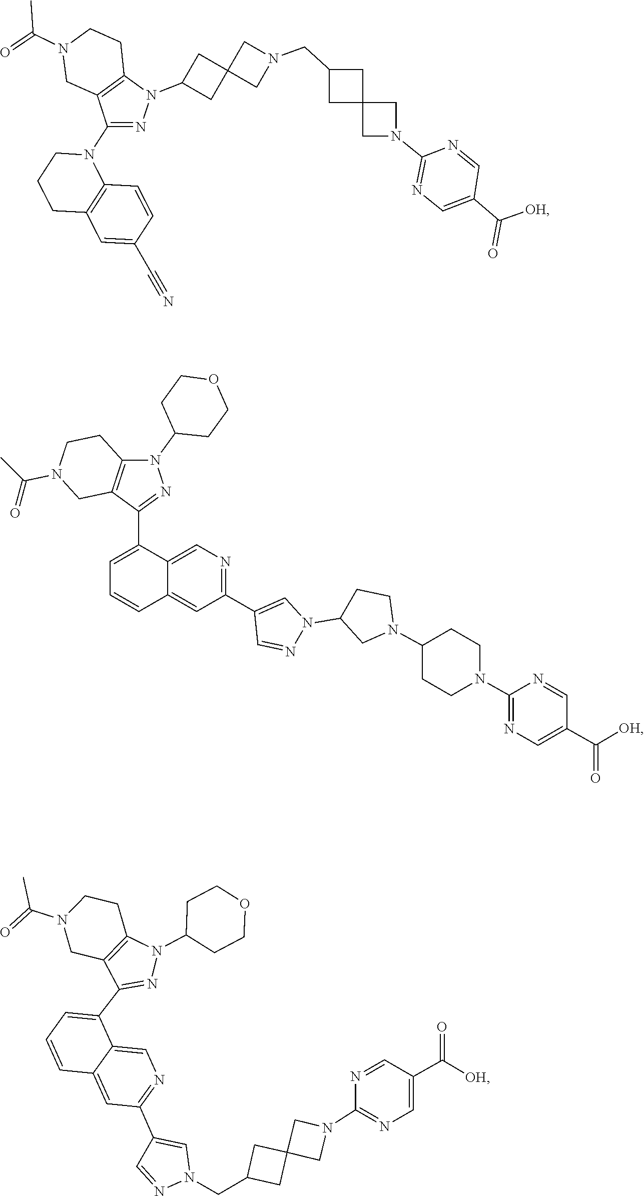 [SMILES: CC(=O)N1CCc2c(c(N3CCCc4cc(C#N)ccc43)nn2C2CC3(C2)CN(CC2CC4(C2)CN(c2ncc(C(=O)O)cn2)C4)C3)C1 ; CC(=O)N1CCc2c(c(-c3cccc4cc(-c5cnn(C6CCN(C7CCN(c8ncc(C(=O)O)cn8)CC7)C6)c5)ncc34)nn2C2CCOCC2)C1 ; CC(=O)N1CCc2c(c(-c3cccc4cc(-c5cnn(CC6CC7(C6)CN(c6ncc(C(=O)O)cn6)C7)c5)ncc34)nn2C2CCOCC2)C1]

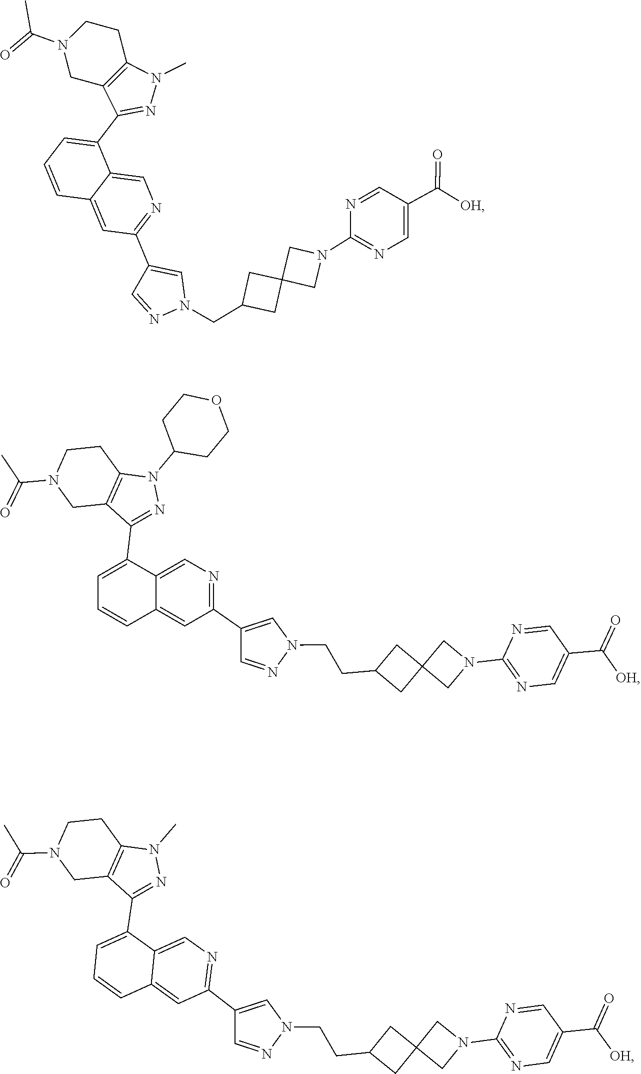 [SMILES: CC(=O)N1CCc2c(c(-c3cccc4cc(-c5cnn(CC6CC7(C6)CN(c6ncc(C(=O)O)cn6)C7)c5)ncc34)nn2C)C1 ; CC(=O)N1CCc2c(c(-c3cccc4cc(-c5cnn(CCC6CC7(C6)CN(c6ncc(C(=O)O)cn6)C7)c5)ncc34)nn2C2CCOCC2)C1 ; CC(=O)N1CCc2c(c(-c3cccc4cc(-c5cnn(CCC6CC7(C6)CN(c6ncc(C(=O)O)cn6)C7)c5)ncc34)nn2C)C1]

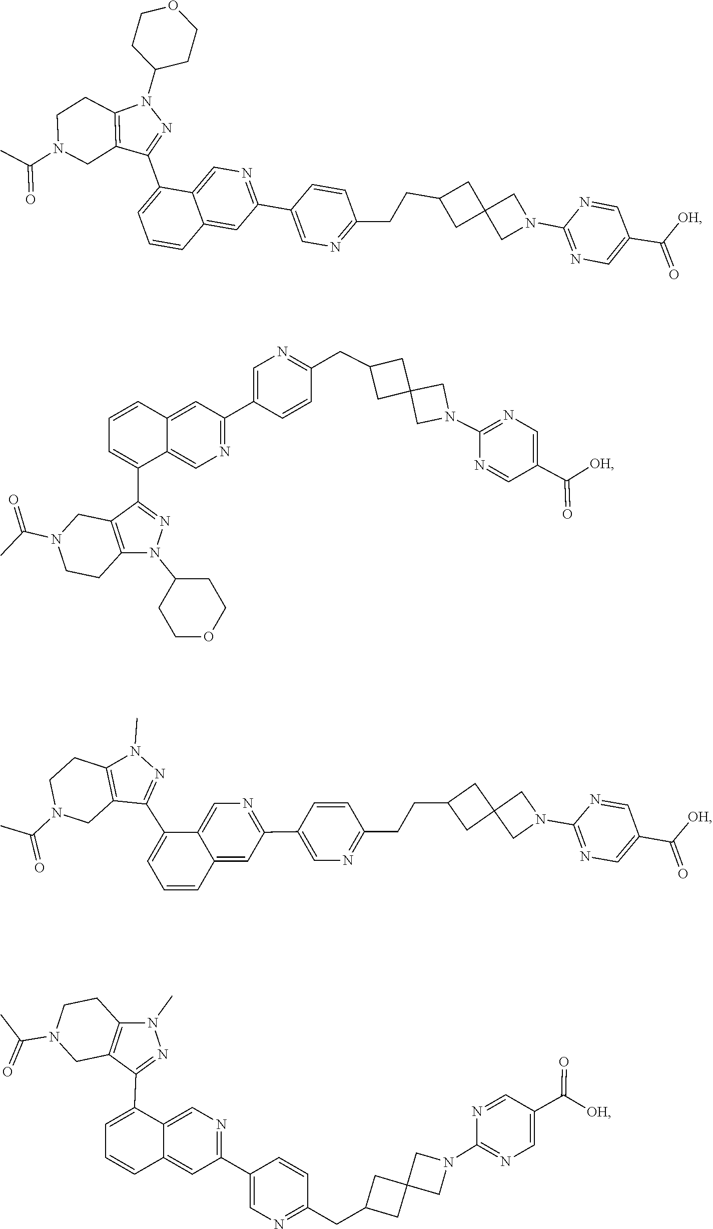 [SMILES: CC(=O)N1CCc2c(c(-c3cccc4cc(-c5ccc(CCC6CC7(C6)CN(c6ncc(C(=O)O)cn6)C7)nc5)ncc34)nn2C2CCOCC2)C1 ; CC(=O)N1CCc2c(c(-c3cccc4cc(-c5ccc(CC6CC7(C6)CN(c6ncc(C(=O)O)cn6)C7)nc5)ncc34)nn2C2CCOCC2)C1 ; CC(=O)N1CCc2c(c(-c3cccc4cc(-c5ccc(CCC6CC7(C6)CN(c6ncc(C(=O)O)cn6)C7)nc5)ncc34)nn2C)C1 ; CC(=O)N1CCc2c(c(-c3cccc4cc(-c5ccc(CC6CC7(C6)CN(c6ncc(C(=O)O)cn6)C7)nc5)ncc34)nn2C)C1]

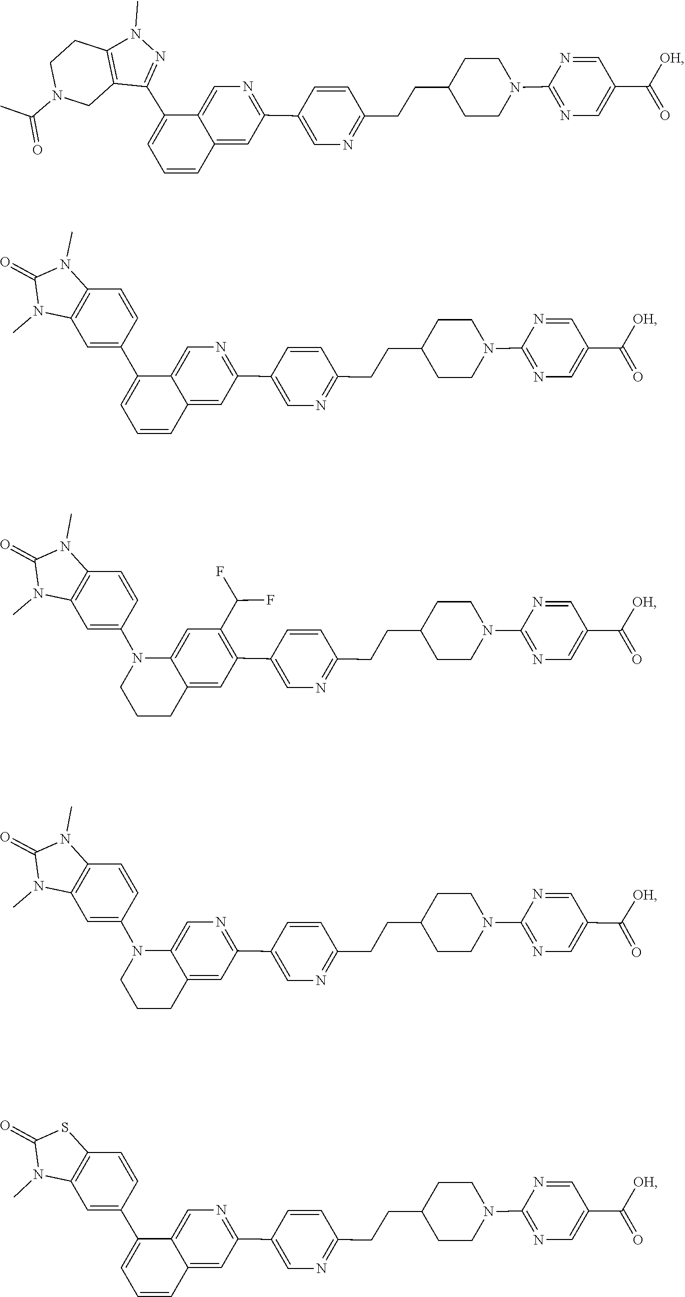 [SMILES: CC(=O)N1CCc2c(c(-c3cccc4cc(-c5ccc(CCC6CCN(c7ncc(C(=O)O)cn7)CC6)nc5)ncc34)nn2C)C1 ; Cn1c(=O)n(C)c2cc(-c3cccc4cc(-c5ccc(CCC6CCN(c7ncc(C(=O)O)cn7)CC6)nc5)ncc34)ccc21 ; Cn1c(=O)n(C)c2cc(N3CCCc4cc(-c5ccc(CCC6CCN(c7ncc(C(=O)O)cn7)CC6)nc5)c(C(F)F)cc43)ccc21 ; Cn1c(=O)n(C)c2cc(N3CCCc4cc(-c5ccc(CCC6CCN(c7ncc(C(=O)O)cn7)CC6)nc5)ncc43)ccc21 ; Cn1c(=O)sc2ccc(-c3cccc4cc(-c5ccc(CCC6CCN(c7ncc(C(=O)O)cn7)CC6)nc5)ncc34)cc21]

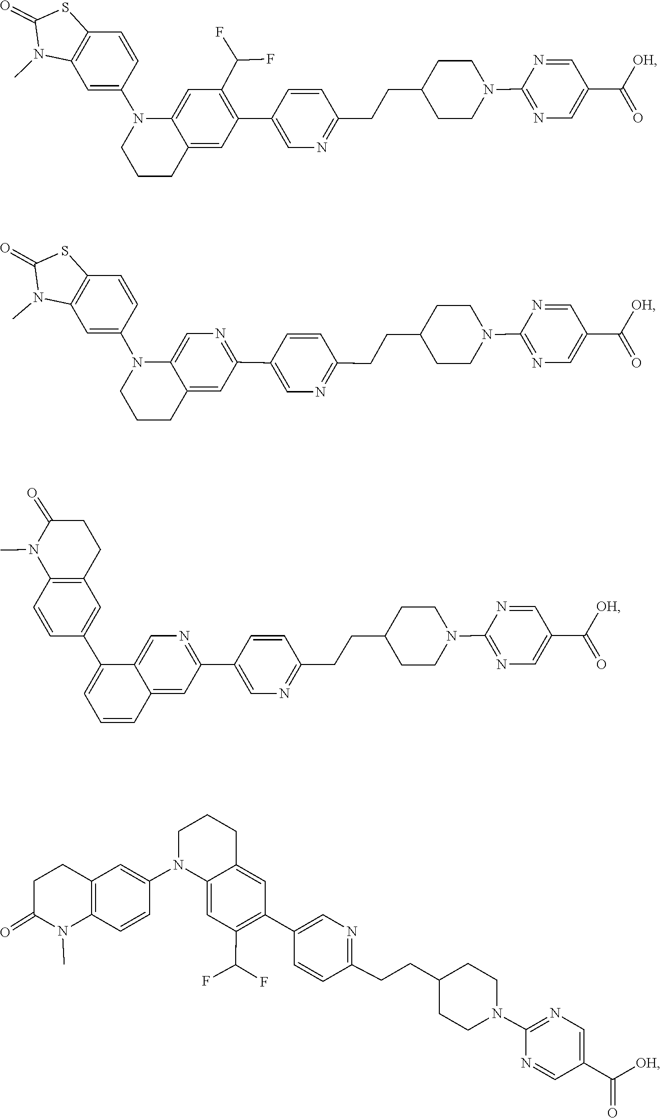 [SMILES: Cn1c(=O)sc2ccc(N3CCCc4cc(-c5ccc(CCC6CCN(c7ncc(C(=O)O)cn7)CC6)nc5)c(C(F)F)cc43)cc21 ; Cn1c(=O)sc2ccc(N3CCCc4cc(-c5ccc(CCC6CCN(c7ncc(C(=O)O)cn7)CC6)nc5)ncc43)cc21 ; CN1C(=O)CCc2cc(-c3cccc4cc(-c5ccc(CCC6CCN(c7ncc(C(=O)O)cn7)CC6)nc5)ncc34)ccc21 ; CN1C(=O)CCc2cc(N3CCCc4cc(-c5ccc(CCC6CCN(c7ncc(C(=O)O)cn7)CC6)nc5)c(C(F)F)cc43)ccc21]

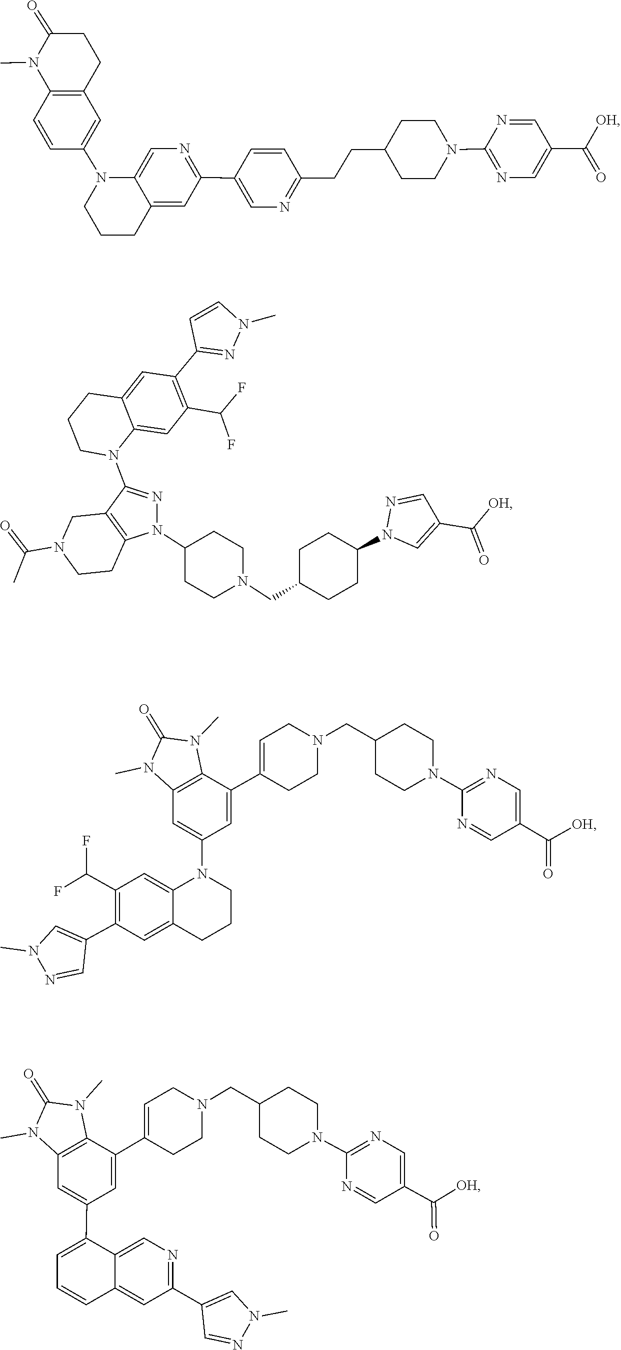 [SMILES: CN1C(=O)CCc2cc(N3CCCc4cc(-c5ccc(CCC6CCN(c7ncc(C(=O)O)cn7)CC6)nc5)ncc43)ccc21 ; CC(=O)N1CCc2c(c(N3CCCc4cc(-c5ccn(C)n5)c(C(F)F)cc43)nn2C2CCN(C[C@H]3CC[C@H](n4cc(C(=O)O)cn4)CC3)CC2)C1 ; Cn1cc(-c2cc3c(cc2C(F)F)N(c2cc(C4=CCN(CC5CCN(c6ncc(C(=O)O)cn6)CC5)CC4)c4c(c2)n(C)c(=O)n4C)CCC3)cn1 ; Cn1cc(-c2cc3cccc(-c4cc(C5=CCN(CC6CCN(c7ncc(C(=O)O)cn7)CC6)CC5)c5c(c4)n(C)c(=O)n5C)c3cn2)cn1]

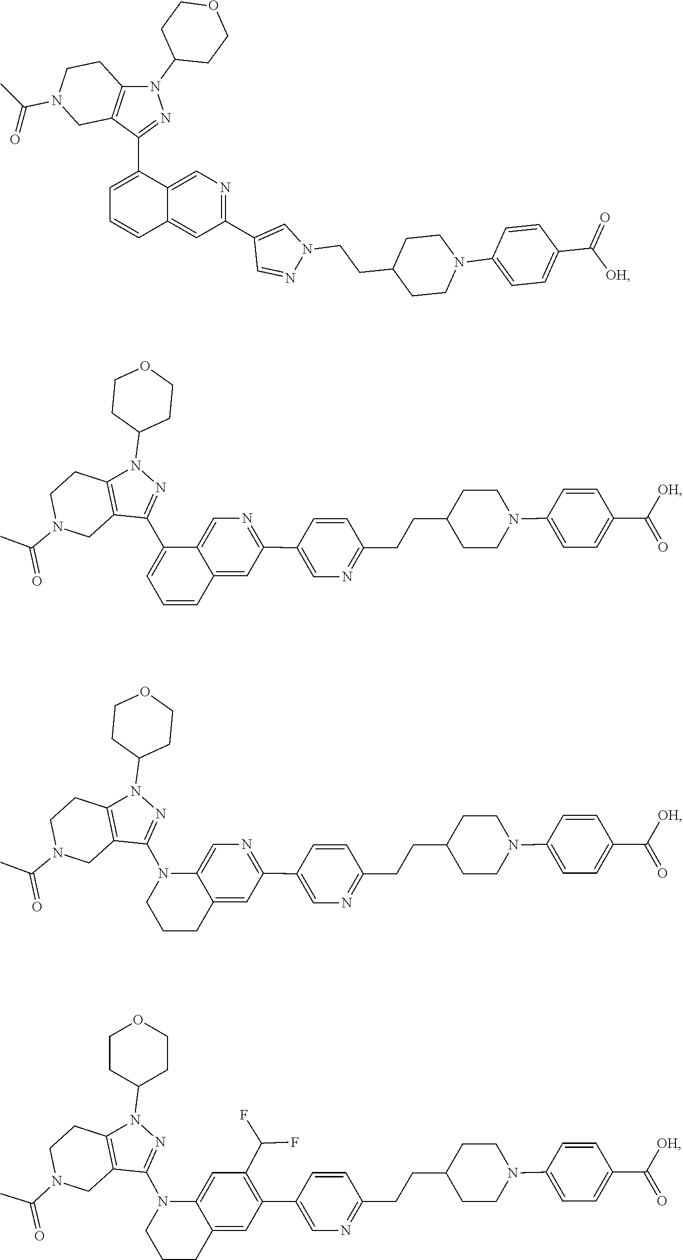 [SMILES: CC(=O)N1CCc2c(c(-c3cccc4cc(-c5cnn(CCC6CCN(c7ccc(C(=O)O)cc7)CC6)c5)ncc34)nn2C2CCOCC2)C1 ; CC(=O)N1CCc2c(c(-c3cccc4cc(-c5ccc(CCC6CCN(c7ccc(C(=O)O)cc7)CC6)nc5)ncc34)nn2C2CCOCC2)C1 ; CC(=O)N1CCc2c(c(N3CCCc4cc(-c5ccc(CCC6CCN(c7ccc(C(=O)O)cc7)CC6)nc5)ncc43)nn2C2CCOCC2)C1 ; CC(=O)N1CCc2c(c(N3CCCc4cc(-c5ccc(CCC6CCN(c7ccc(C(=O)O)cc7)CC6)nc5)c(C(F)F)cc43)nn2C2CCOCC2)C1]

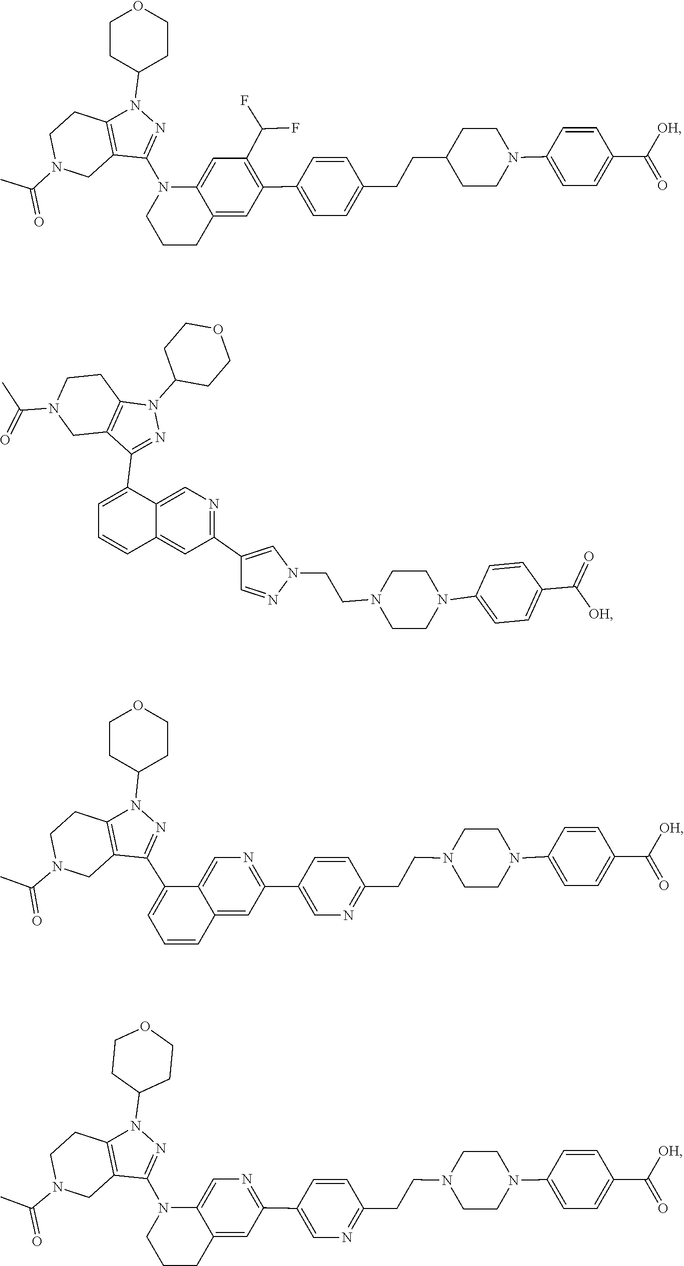 [SMILES: CC(=O)N1CCc2c(c(N3CCCc4cc(-c5ccc(CCC6CCN(c7ccc(C(=O)O)cc7)CC6)cc5)c(C(F)F)cc43)nn2C2CCOCC2)C1 ; CC(=O)N1CCc2c(c(-c3cccc4cc(-c5cnn(CCN6CCN(c7ccc(C(=O)O)cc7)CC6)c5)ncc34)nn2C2CCOCC2)C1 ; CC(=O)N1CCc2c(c(-c3cccc4cc(-c5ccc(CCN6CCN(c7ccc(C(=O)O)cc7)CC6)nc5)ncc34)nn2C2CCOCC2)C1 ; CC(=O)N1CCc2c(c(N3CCCc4cc(-c5ccc(CCN6CCN(c7ccc(C(=O)O)cc7)CC6)nc5)ncc43)nn2C2CCOCC2)C1]

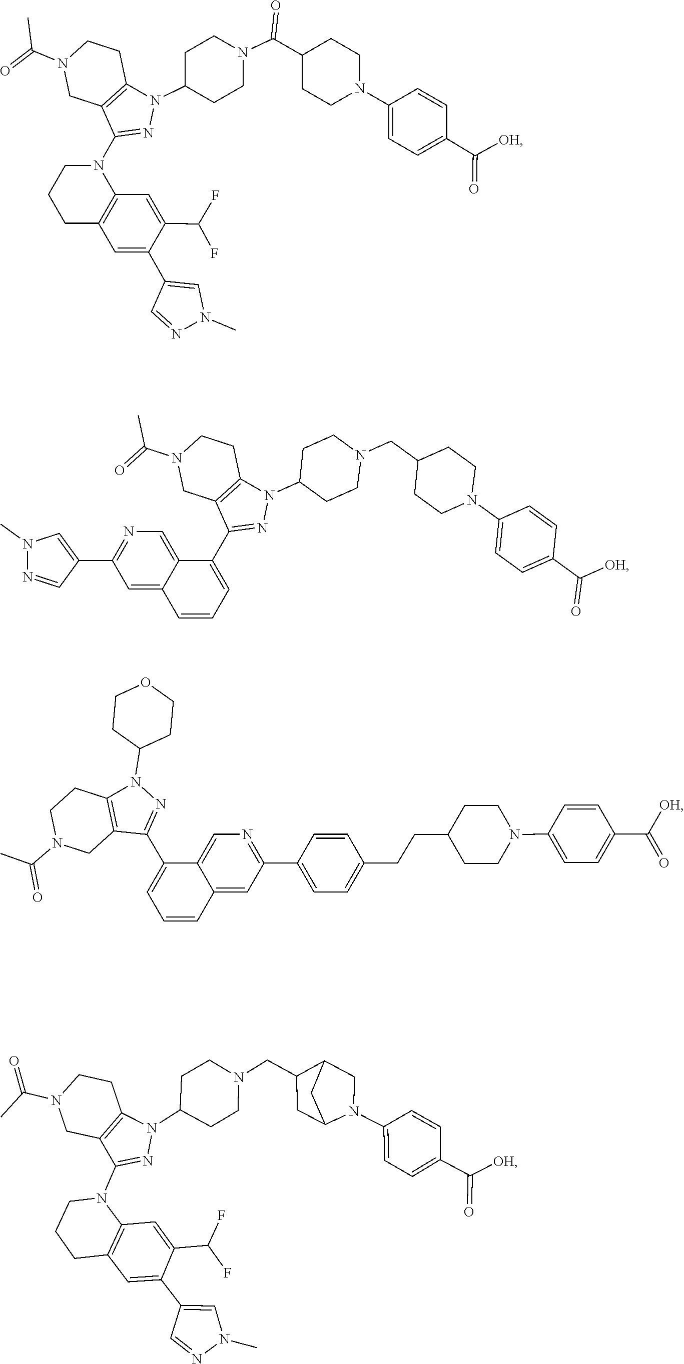 [SMILES: CC(=O)N1CCc2c(c(N3CCCc4cc(-c5cnn(C)c5)c(C(F)F)cc43)nn2C2CCN(C(=O)C3CCN(c4ccc(C(=O)O)cc4)CC3)CC2)C1 ; CC(=O)N1CCc2c(c(-c3cccc4cc(-c5cnn(C)c5)ncc34)nn2C2CCN(CC3CCN(c4ccc(C(=O)O)cc4)CC3)CC2)C1 ; CC(=O)N1CCc2c(c(-c3cccc4cc(-c5ccc(CCC6CCN(c7ccc(C(=O)O)cc7)CC6)cc5)ncc34)nn2C2CCOCC2)C1 ; CC(=O)N1CCc2c(c(N3CCCc4cc(-c5cnn(C)c5)c(C(F)F)cc43)nn2C2CCN(CC3CC4CC3CN4c3ccc(C(=O)O)cc3)CC2)C1]

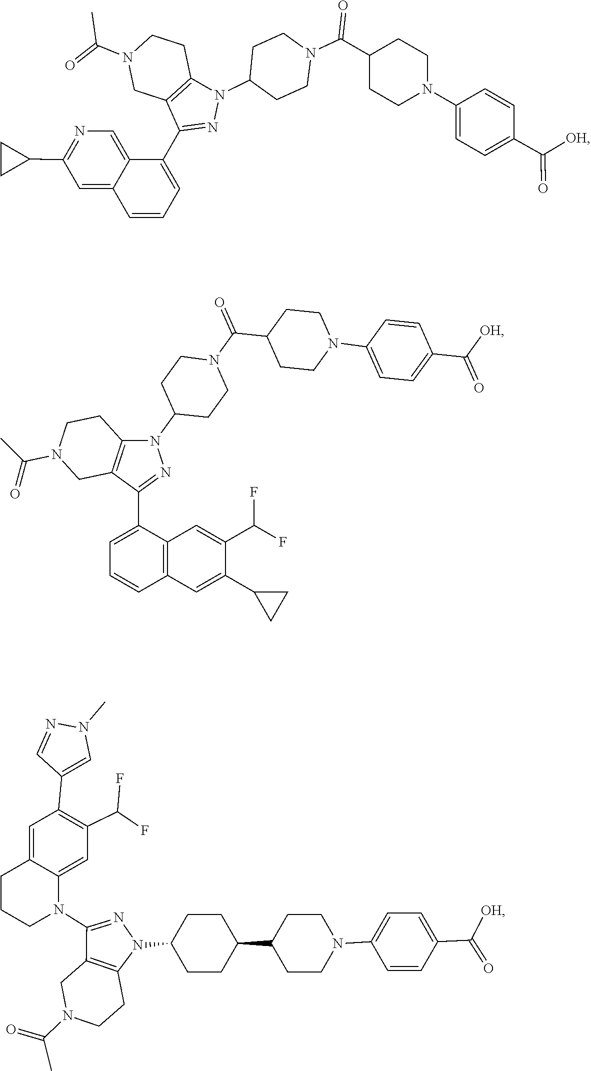 [SMILES: CC(=O)N1CCc2c(c(-c3cccc4cc(C5CC5)ncc34)nn2C2CCN(C(=O)C3CCN(c4ccc(C(=O)O)cc4)CC3)CC2)C1 ; CC(=O)N1CCc2c(c(-c3cccc4cc(C5CC5)c(C(F)F)cc34)nn2C2CCN(C(=O)C3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@H](C3CCN(c4ccc(C(=O)O)cc4)CC3)CC2)C1]

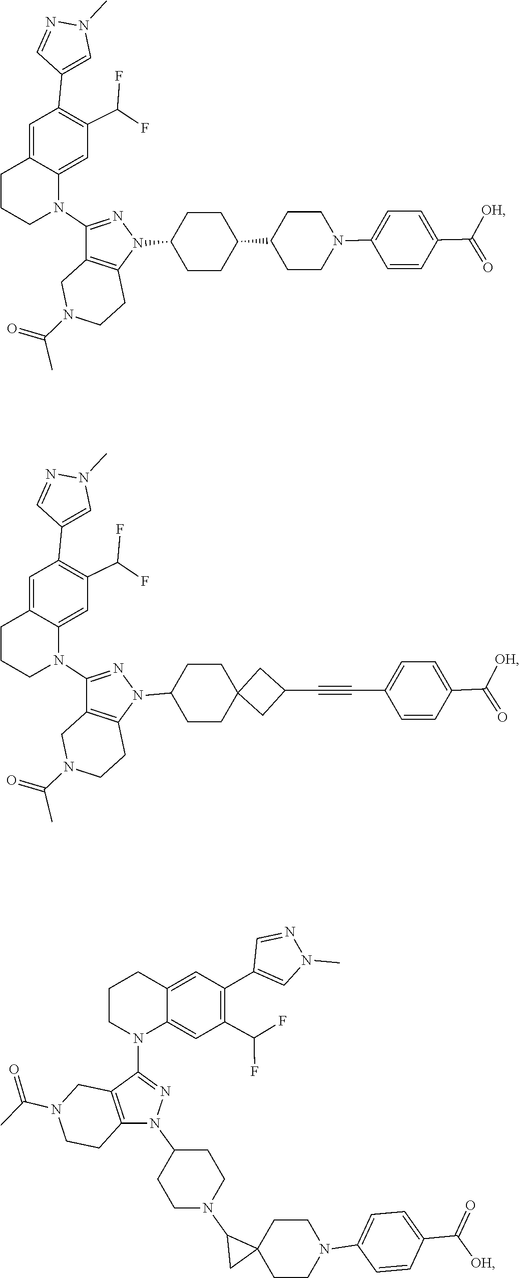 [SMILES: CC(=O)N1CCc2c(c(N3CCCc4cc(-c5cnn(C)c5)c(C(F)F)cc43)nn2[C@H]2CC[C@@H](C3CCN(c4ccc(C(=O)O)cc4)CC3)CC2)C1 ; CC(=O)N1CCc2c(c(N3CCCc4cc(-c5cnn(C)c5)c(C(F)F)cc43)nn2C2CCC3(CC2)CC(C#Cc2ccc(C(=O)O)cc2)C3)C1 ; CC(=O)N1CCc2c(c(N3CCCc4cc(-c5cnn(C)c5)c(C(F)F)cc43)nn2C2CCN(C3CC34CCN(c3ccc(C(=O)O)cc3)CC4)CC2)C1]

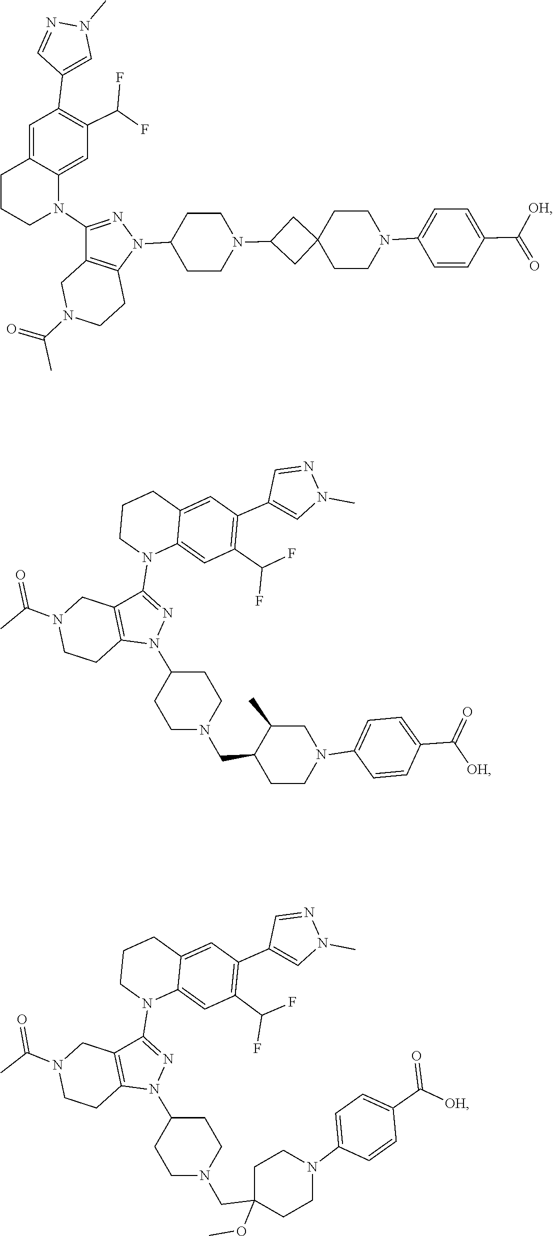 [SMILES: CC(=O)N1CCc2c(c(N3CCCc4cc(-c5cnn(C)c5)c(C(F)F)cc43)nn2C2CCN(C3CC4(CCN(c5ccc(C(=O)O)cc5)CC4)C3)CC2)C1 ; CC(=O)N1CCc2c(c(N3CCCc4cc(-c5cnn(C)c5)c(C(F)F)cc43)nn2C2CCN(C[C@@H]3CCN(c4ccc(C(=O)O)cc4)C[C@@H]3C)CC2)C1 ; COC1(CN2CCC(n3nc(N4CCCc5cc(-c6cnn(C)c6)c(C(F)F)cc54)c4c3CCN(C(C)=O)C4)CC2)CCN(c2ccc(C(=O)O)cc2)CC1]

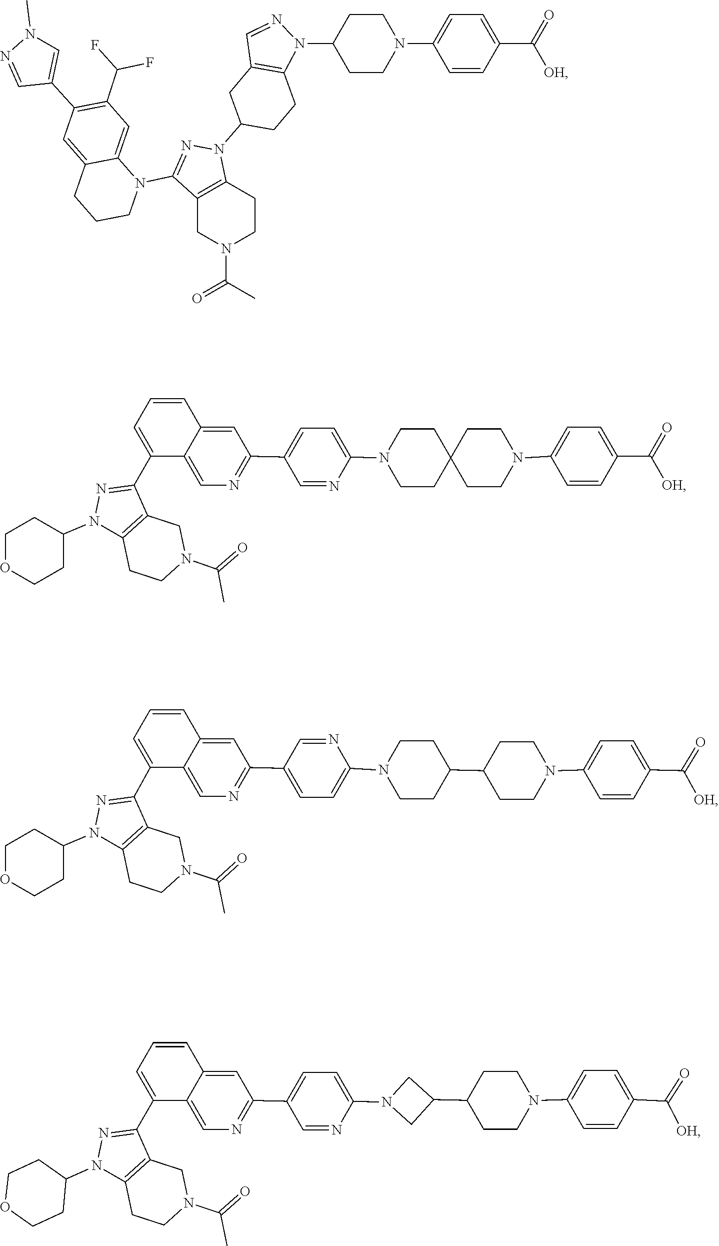 [SMILES: CC(=O)N1CCc2c(c(N3CCCc4cc(-c5cnn(C)c5)c(C(F)F)cc43)nn2C2CCc3c(cnn3C3CCN(c4ccc(C(=O)O)cc4)CC3)C2)C1 ; CC(=O)N1CCc2c(c(-c3cccc4cc(-c5ccc(N6CCC7(CCN(c8ccc(C(=O)O)cc8)CC7)CC6)nc5)ncc34)nn2C2CCOCC2)C1 ; CC(=O)N1CCc2c(c(-c3cccc4cc(-c5ccc(N6CCC(C7CCN(c8ccc(C(=O)O)cc8)CC7)CC6)nc5)ncc34)nn2C2CCOCC2)C1 ; CC(=O)N1CCc2c(c(-c3cccc4cc(-c5ccc(N6CC(C7CCN(c8ccc(C(=O)O)cc8)CC7)C6)nc5)ncc34)nn2C2CCOCC2)C1]

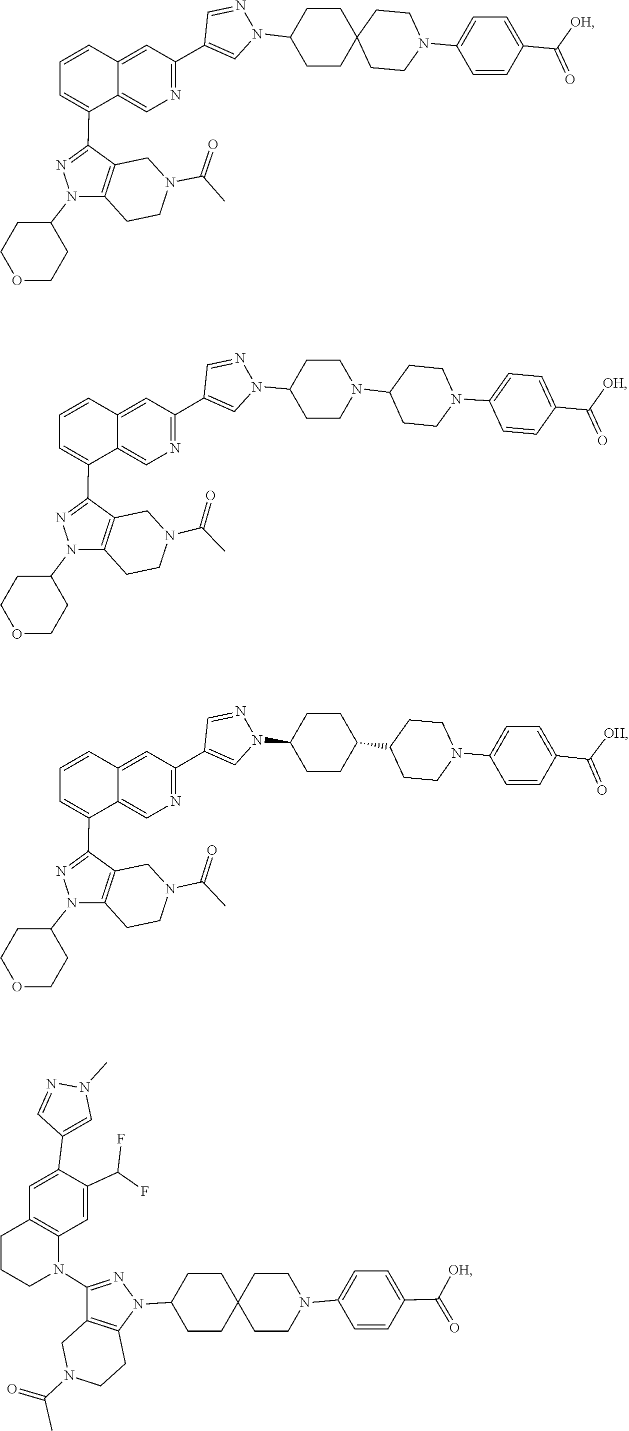 [SMILES: CC(=O)N1CCc2c(c(-c3cccc4cc(-c5cnn(C6CCC7(CC6)CCN(c6ccc(C(=O)O)cc6)CC7)c5)ncc34)nn2C2CCOCC2)C1 ; CC(=O)N1CCc2c(c(-c3cccc4cc(-c5cnn(C6CCN(C7CCN(c8ccc(C(=O)O)cc8)CC7)CC6)c5)ncc34)nn2C2CCOCC2)C1 ; CC(=O)N1CCc2c(c(-c3cccc4cc(-c5cnn([C@H]6CC[C@H](C7CCN(c8ccc(C(=O)O)cc8)CC7)CC6)c5)ncc34)nn2C2CCOCC2)C1 ; CC(=O)N1CCc2c(c(N3CCCc4cc(-c5cnn(C)c5)c(C(F)F)cc43)nn2C2CCC3(CC2)CCN(c2ccc(C(=O)O)cc2)CC3)C1]

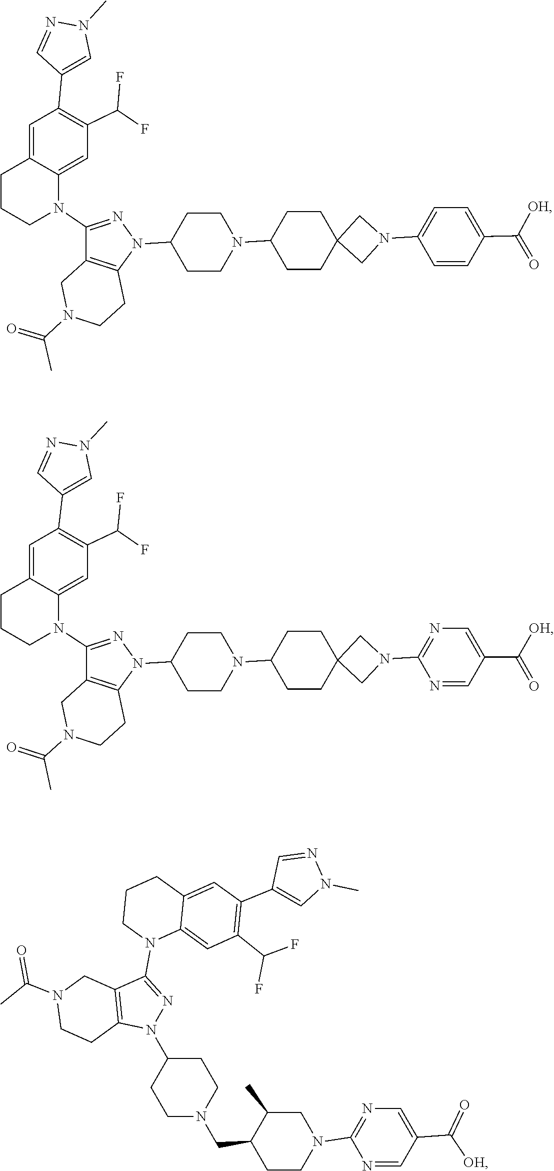 [SMILES: CC(=O)N1CCc2c(c(N3CCCc4cc(-c5cnn(C)c5)c(C(F)F)cc43)nn2C2CCN(C3CCC4(CC3)CN(c3ccc(C(=O)O)cc3)C4)CC2)C1 ; CC(=O)N1CCc2c(c(N3CCCc4cc(-c5cnn(C)c5)c(C(F)F)cc43)nn2C2CCN(C3CCC4(CC3)CN(c3ncc(C(=O)O)cn3)C4)CC2)C1 ; CC(=O)N1CCc2c(c(N3CCCc4cc(-c5cnn(C)c5)c(C(F)F)cc43)nn2C2CCN(C[C@@H]3CCN(c4ncc(C(=O)O)cn4)C[C@@H]3C)CC2)C1]

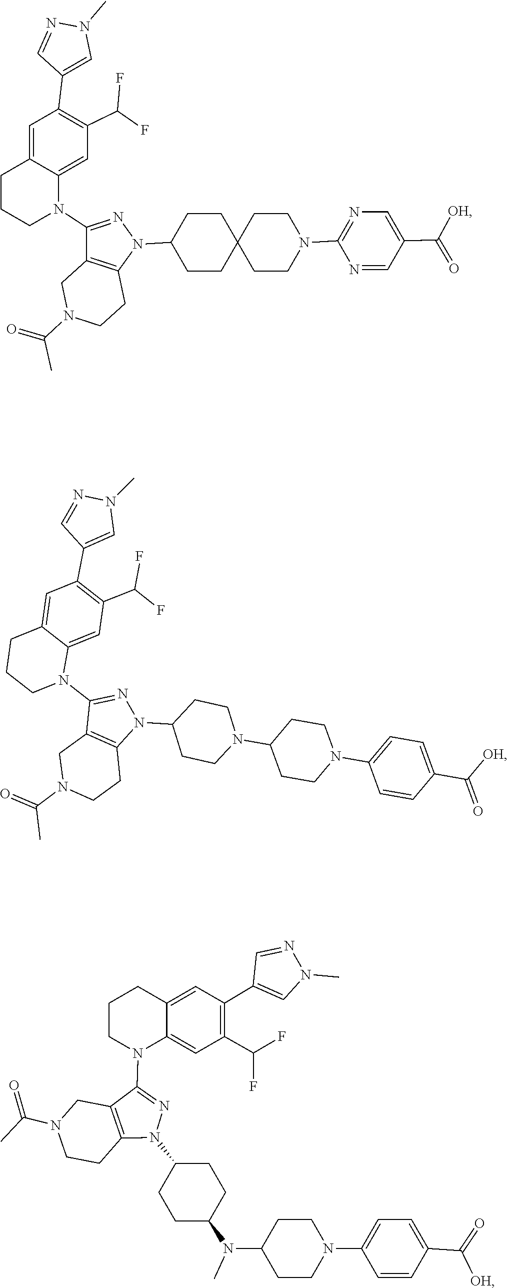 [SMILES: CC(=O)N1CCc2c(c(N3CCCc4cc(-c5cnn(C)c5)c(C(F)F)cc43)nn2C2CCC3(CC2)CCN(c2ncc(C(=O)O)cn2)CC3)C1 ; CC(=O)N1CCc2c(c(N3CCCc4cc(-c5cnn(C)c5)c(C(F)F)cc43)nn2C2CCN(C3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@H](N(C)C3CCN(c4ccc(C(=O)O)cc4)CC3)CC2)C1]

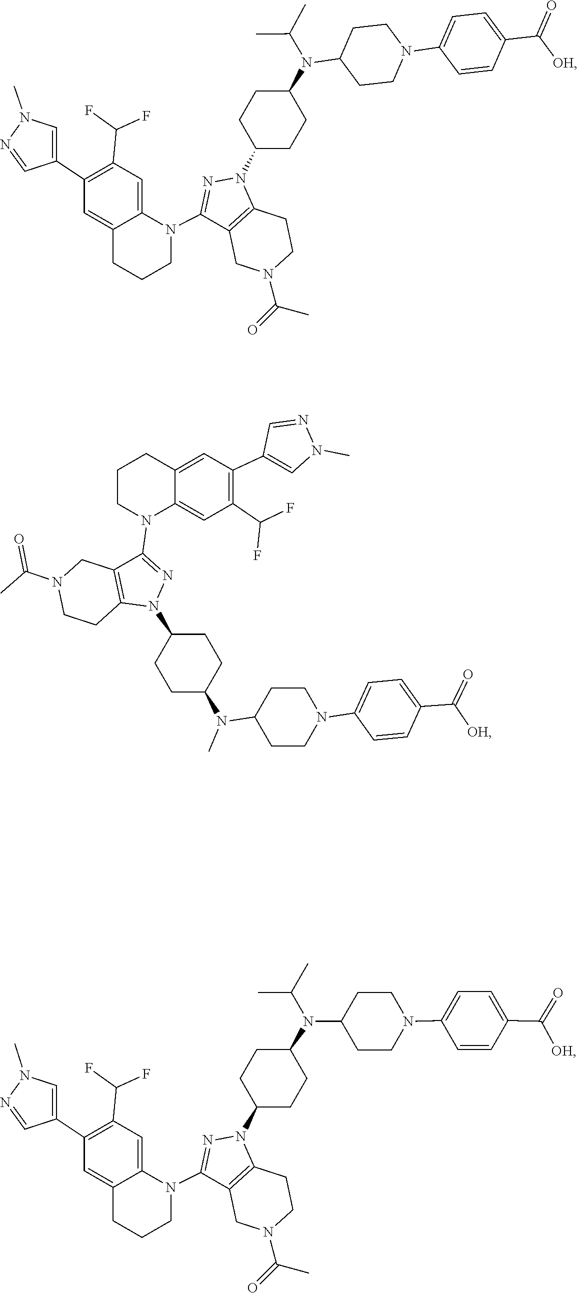 [SMILES: CC(=O)N1CCc2c(c(N3CCCc4cc(-c5cnn(C)c5)c(C(F)F)cc43)nn2[C@H]2CC[C@H](N(C(C)C)C3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@@H](N(C)C3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@@H](N(C(C)C)C3CCN(c4ccc(C(=O)O)cc4)CC3)CC2)C1]

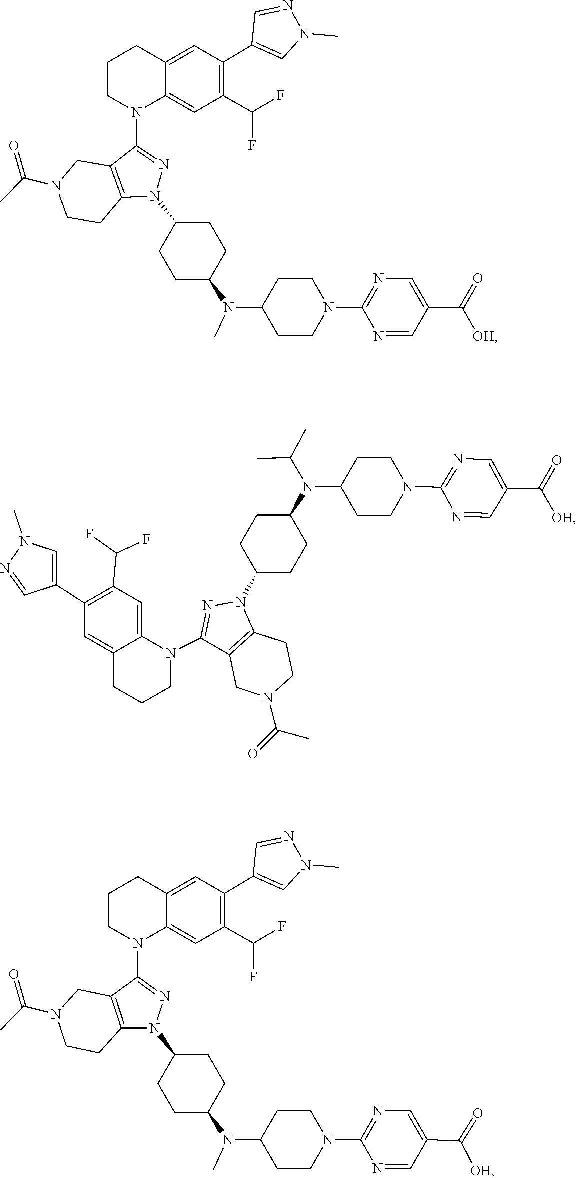 [SMILES: CC(=O)N1CCc2c(c(N3CCCc4cc(-c5cnn(C)c5)c(C(F)F)cc43)nn2[C@H]2CC[C@H](N(C)C3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@H](N(C(C)C)C3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@@H](N(C)C3CCN(c4ncc(C(=O)O)cn4)CC3)CC2)C1]

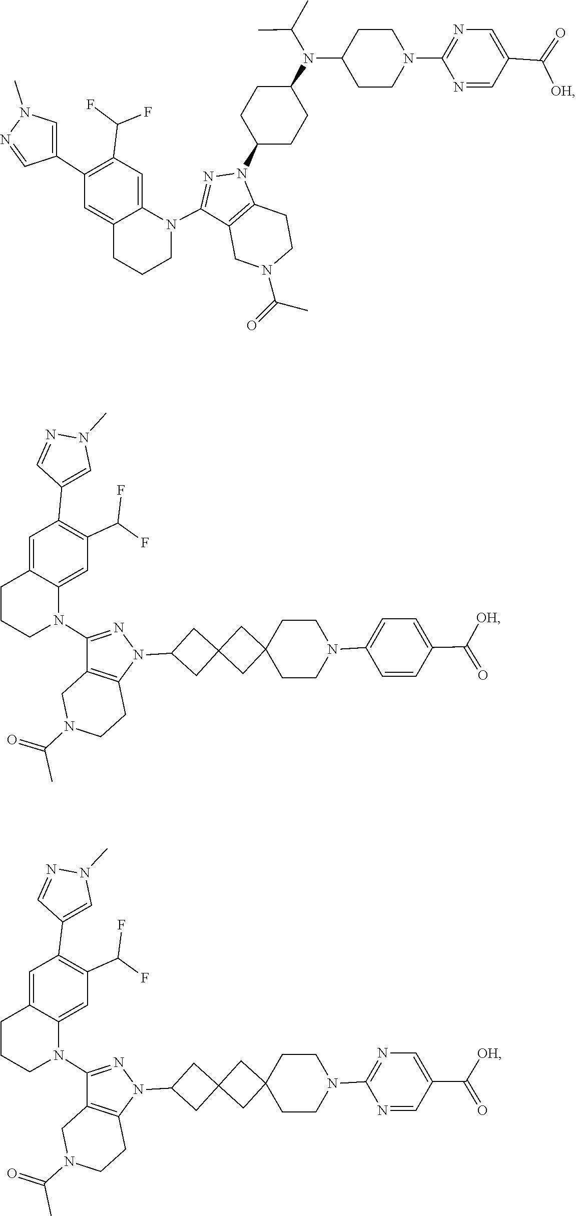 [SMILES: CC(=O)N1CCc2c(c(N3CCCc4cc(-c5cnn(C)c5)c(C(F)F)cc43)nn2[C@H]2CC[C@@H](N(C(C)C)C3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CC3(C2)CC2(CCN(c4ccc(C(=O)O)cc4)CC2)C3)C1 ; CC(=O)N1CCc2c(c(N3CCCc4cc(-c5cnn(C)c5)c(C(F)F)cc43)nn2C2CC3(C2)CC2(CCN(c4ncc(C(=O)O)cn4)CC2)C3)C1]

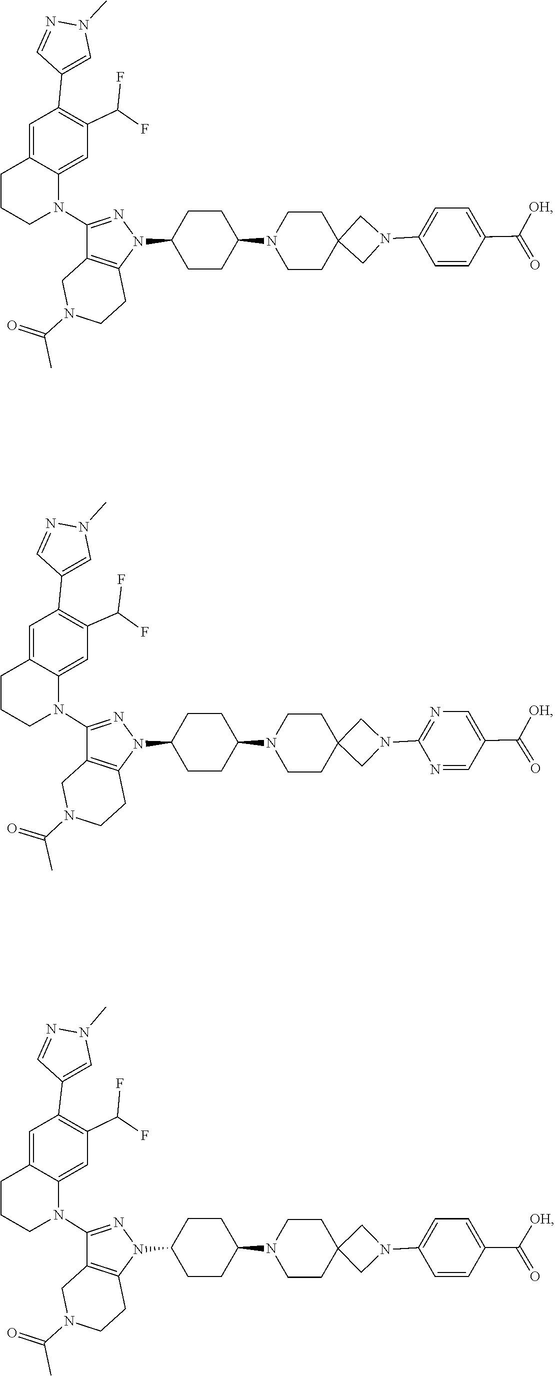 [SMILES: CC(=O)N1CCc2c(c(N3CCCc4cc(-c5cnn(C)c5)c(C(F)F)cc43)nn2[C@H]2CC[C@@H](N3CCC4(CC3)CN(c3ccc(C(=O)O)cc3)C4)CC2)C1 ; CC(=O)N1CCc2c(c(N3CCCc4cc(-c5cnn(C)c5)c(C(F)F)cc43)nn2[C@H]2CC[C@@H](N3CCC4(CC3)CN(c3ncc(C(=O)O)cn3)C4)CC2)C1 ; CC(=O)N1CCc2c(c(N3CCCc4cc(-c5cnn(C)c5)c(C(F)F)cc43)nn2[C@H]2CC[C@H](N3CCC4(CC3)CN(c3ccc(C(=O)O)cc3)C4)CC2)C1]

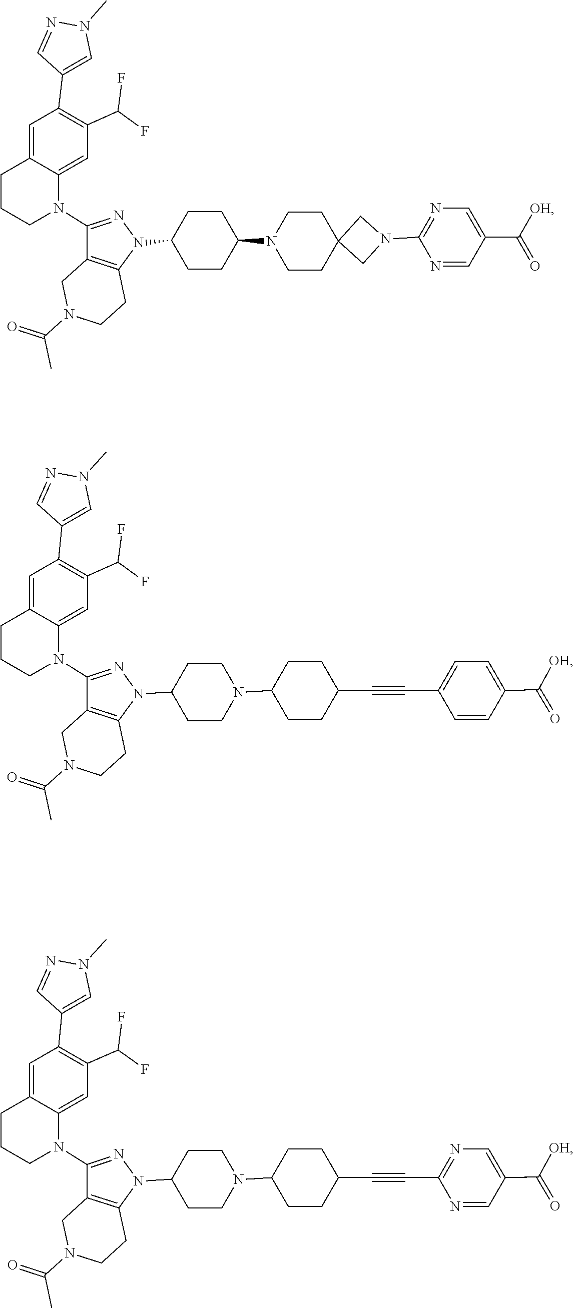 [SMILES: CC(=O)N1CCc2c(c(N3CCCc4cc(-c5cnn(C)c5)c(C(F)F)cc43)nn2[C@H]2CC[C@H](N3CCC4(CC3)CN(c3ncc(C(=O)O)cn3)C4)CC2)C1 ; CC(=O)N1CCc2c(c(N3CCCc4cc(-c5cnn(C)c5)c(C(F)F)cc43)nn2C2CCN(C3CCC(C#Cc4ccc(C(=O)O)cc4)CC3)CC2)C1 ; CC(=O)N1CCc2c(c(N3CCCc4cc(-c5cnn(C)c5)c(C(F)F)cc43)nn2C2CCN(C3CCC(C#Cc4ncc(C(=O)O)cn4)CC3)CC2)C1]

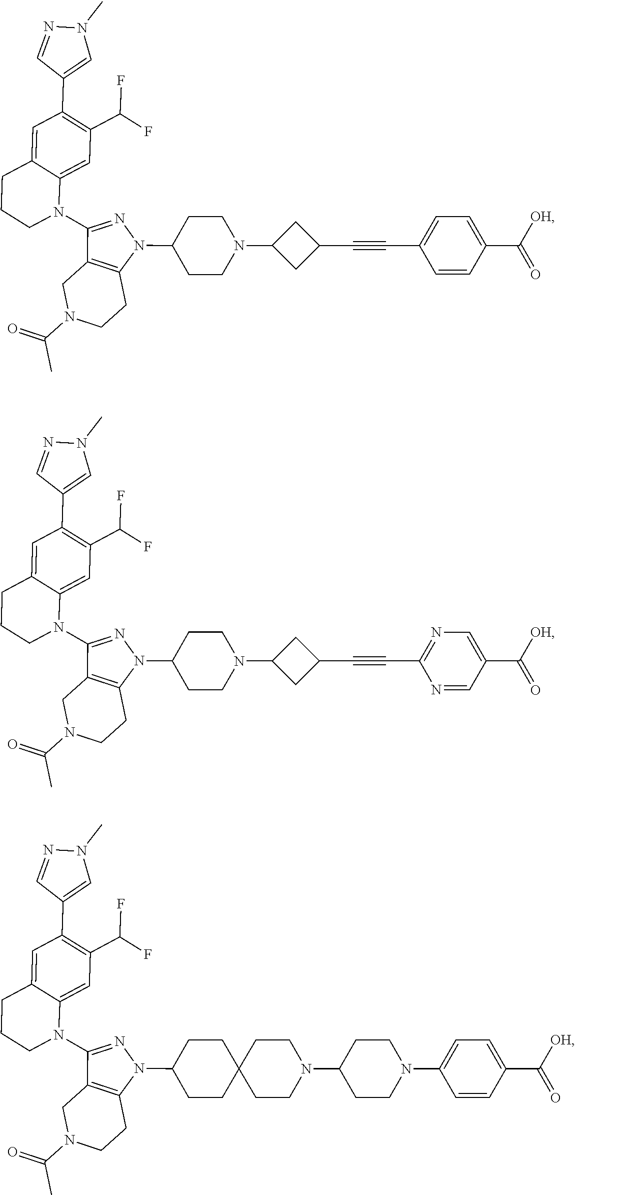 [SMILES: CC(=O)N1CCc2c(c(N3CCCc4cc(-c5cnn(C)c5)c(C(F)F)cc43)nn2C2CCN(C3CC(C#Cc4ccc(C(=O)O)cc4)C3)CC2)C1 ; CC(=O)N1CCc2c(c(N3CCCc4cc(-c5cnn(C)c5)c(C(F)F)cc43)nn2C2CCN(C3CC(C#Cc4ncc(C(=O)O)cn4)C3)CC2)C1 ; CC(=O)N1CCc2c(c(N3CCCc4cc(-c5cnn(C)c5)c(C(F)F)cc43)nn2C2CCC3(CC2)CCN(C2CCN(c4ccc(C(=O)O)cc4)CC2)CC3)C1]

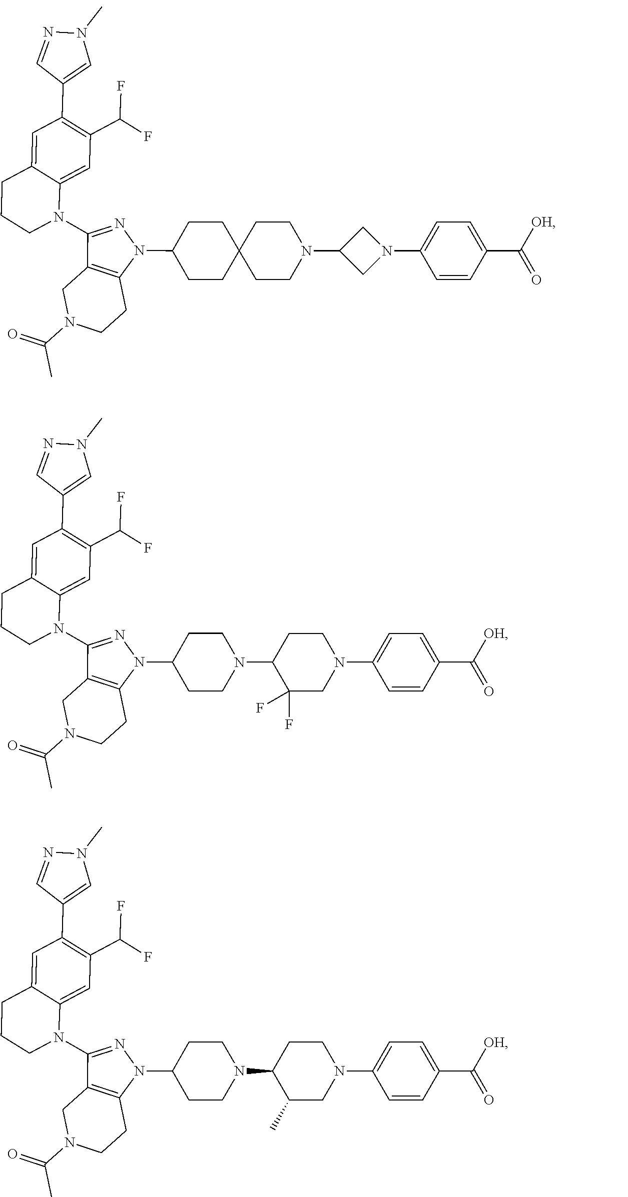 [SMILES: CC(=O)N1CCc2c(c(N3CCCc4cc(-c5cnn(C)c5)c(C(F)F)cc43)nn2C2CCC3(CC2)CCN(C2CN(c4ccc(C(=O)O)cc4)C2)CC3)C1 ; CC(=O)N1CCc2c(c(N3CCCc4cc(-c5cnn(C)c5)c(C(F)F)cc43)nn2C2CCN(C3CCN(c4ccc(C(=O)O)cc4)CC3(F)F)CC2)C1 ; CC(=O)N1CCc2c(c(N3CCCc4cc(-c5cnn(C)c5)c(C(F)F)cc43)nn2C2CCN([C@H]3CCN(c4ccc(C(=O)O)cc4)C[C@@H]3C)CC2)C1]

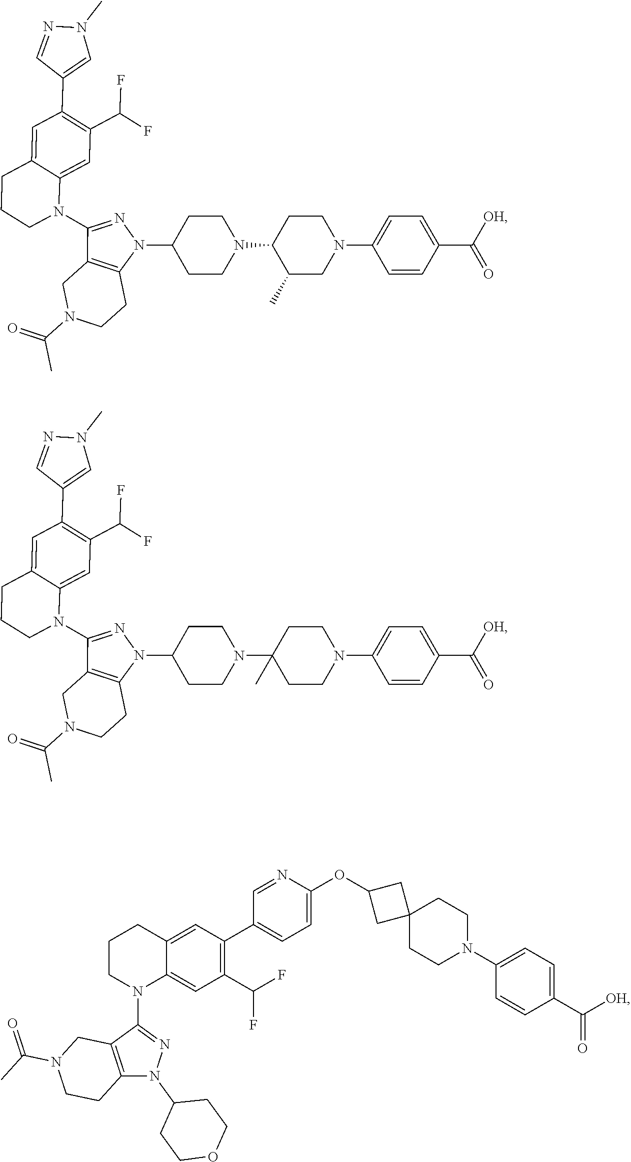 [SMILES: CC(=O)N1CCc2c(c(N3CCCc4cc(-c5cnn(C)c5)c(C(F)F)cc43)nn2C2CCN([C@@H]3CCN(c4ccc(C(=O)O)cc4)C[C@@H]3C)CC2)C1 ; CC(=O)N1CCc2c(c(N3CCCc4cc(-c5cnn(C)c5)c(C(F)F)cc43)nn2C2CCN(C3(C)CCN(c4ccc(C(=O)O)cc4)CC3)CC2)C1 ; CC(=O)N1CCc2c(c(N3CCCc4cc(-c5ccc(OC6CC7(CCN(c8ccc(C(=O)O)cc8)CC7)C6)nc5)c(C(F)F)cc43)nn2C2CCOCC2)C1]

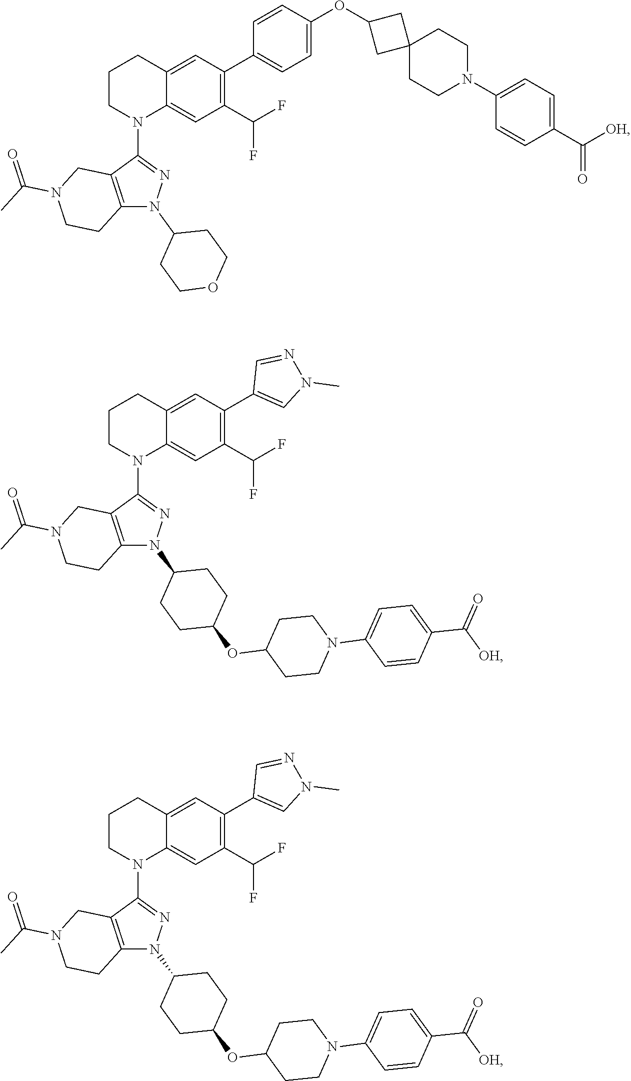 [SMILES: CC(=O)N1CCc2c(c(N3CCCc4cc(-c5ccc(OC6CC7(CCN(c8ccc(C(=O)O)cc8)CC7)C6)cc5)c(C(F)F)cc43)nn2C2CCOCC2)C1 ; CC(=O)N1CCc2c(c(N3CCCc4cc(-c5cnn(C)c5)c(C(F)F)cc43)nn2[C@H]2CC[C@@H](OC3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@H](OC3CCN(c4ccc(C(=O)O)cc4)CC3)CC2)C1]

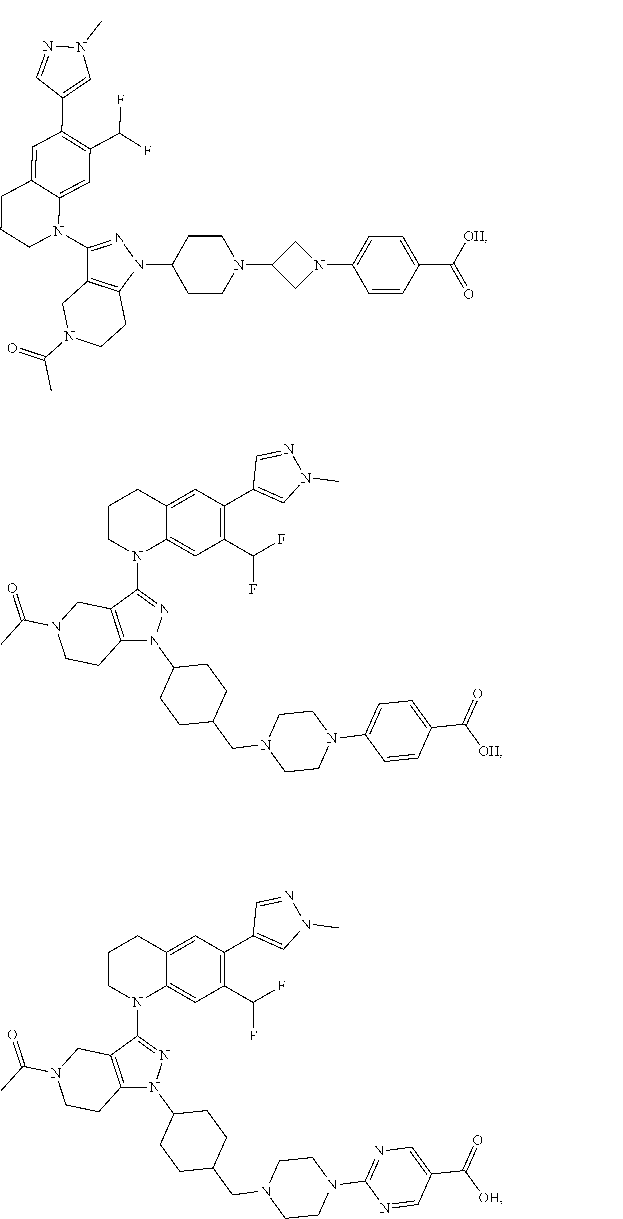 [SMILES: CC(=O)N1CCc2c(c(N3CCCc4cc(-c5cnn(C)c5)c(C(F)F)cc43)nn2C2CCN(C3CN(c4ccc(C(=O)O)cc4)C3)CC2)C1 ; CC(=O)N1CCc2c(c(N3CCCc4cc(-c5cnn(C)c5)c(C(F)F)cc43)nn2C2CCC(CN3CCN(c4ccc(C(=O)O)cc4)CC3)CC2)C1 ; CC(=O)N1CCc2c(c(N3CCCc4cc(-c5cnn(C)c5)c(C(F)F)cc43)nn2C2CCC(CN3CCN(c4ncc(C(=O)O)cn4)CC3)CC2)C1]

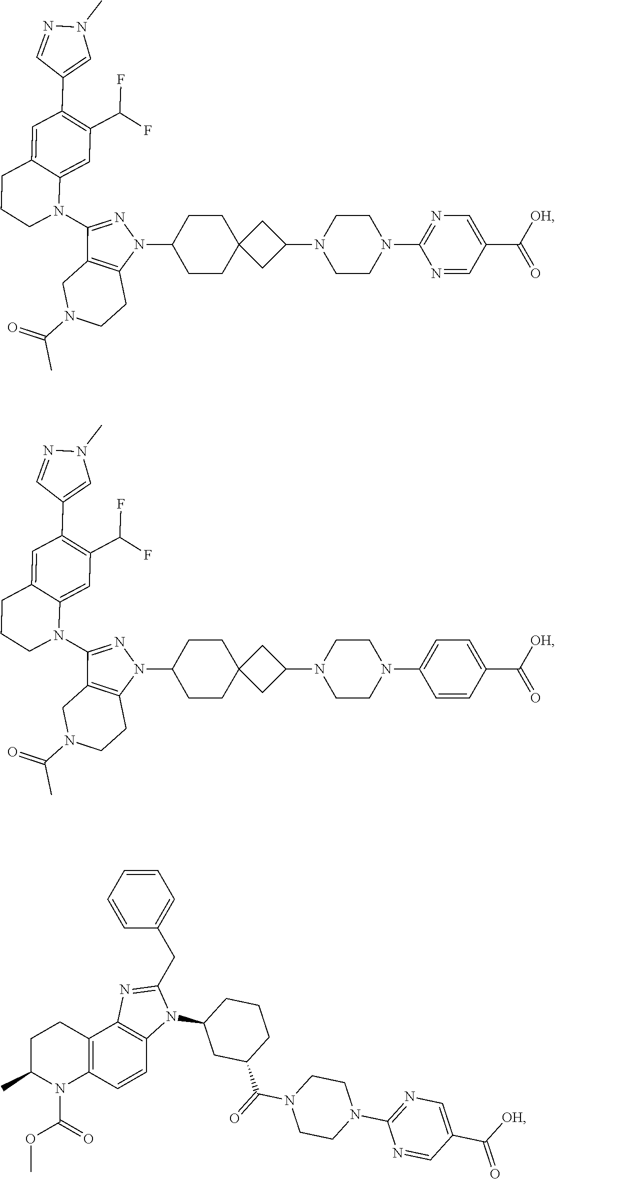 [SMILES: CC(=O)N1CCc2c(c(N3CCCc4cc(-c5cnn(C)c5)c(C(F)F)cc43)nn2C2CCC3(CC2)CC(N2CCN(c4ncc(C(=O)O)cn4)CC2)C3)C1 ; CC(=O)N1CCc2c(c(N3CCCc4cc(-c5cnn(C)c5)c(C(F)F)cc43)nn2C2CCC3(CC2)CC(N2CCN(c4ccc(C(=O)O)cc4)CC2)C3)C1 ; COC(=O)N1c2ccc3c(nc(Cc4ccccc4)n3[C@H]3CCC[C@H](C(=O)N4CCN(c5ncc(C(=O)O)cn5)CC4)C3)c2CC[C@@H]1C]

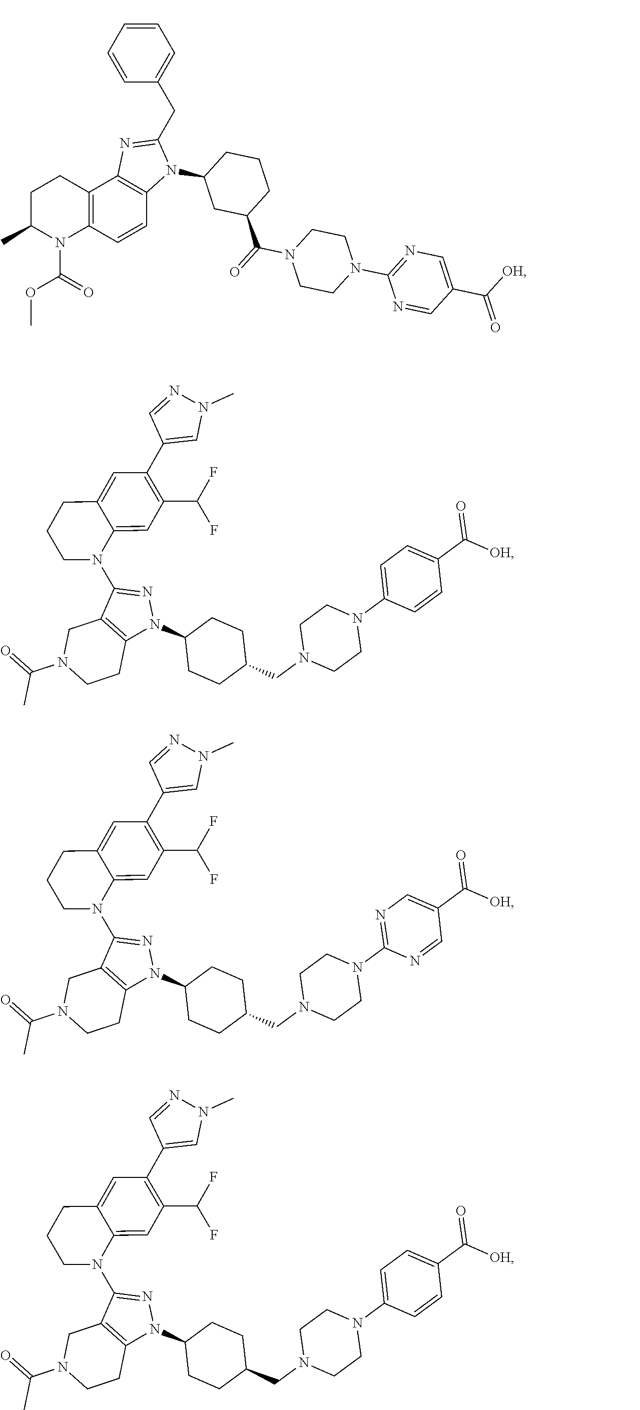 [SMILES: COC(=O)N1c2ccc3c(nc(Cc4ccccc4)n3[C@H]3CCC[C@@H](C(=O)N4CCN(c5ncc(C(=O)O)cn5)CC4)C3)c2CC[C@@H]1C ; CC(=O)N1CCc2c(c(N3CCCc4cc(-c5cnn(C)c5)c(C(F)F)cc43)nn2[C@H]2CC[C@H](CN3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@H](CN3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@@H](CN3CCN(c4ccc(C(=O)O)cc4)CC3)CC2)C1]

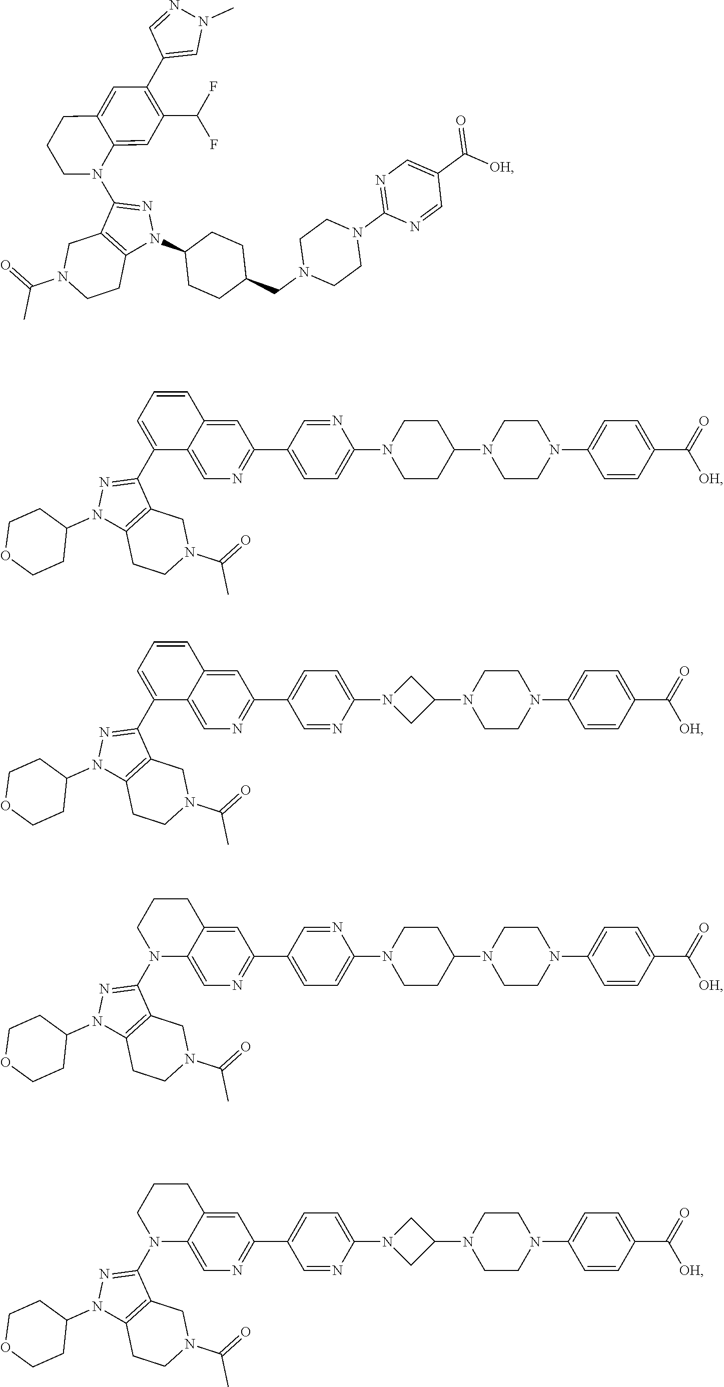 [SMILES: CC(=O)N1CCc2c(c(N3CCCc4cc(-c5cnn(C)c5)c(C(F)F)cc43)nn2[C@H]2CC[C@@H](CN3CCN(c4ncc(C(=O)O)cn4)CC3)CC2)C1 ; CC(=O)N1CCc2c(c(-c3cccc4cc(-c5ccc(N6CCC(N7CCN(c8ccc(C(=O)O)cc8)CC7)CC6)nc5)ncc34)nn2C2CCOCC2)C1 ; CC(=O)N1CCc2c(c(-c3cccc4cc(-c5ccc(N6CC(N7CCN(c8ccc(C(=O)O)cc8)CC7)C6)nc5)ncc34)nn2C2CCOCC2)C1 ; CC(=O)N1CCc2c(c(N3CCCc4cc(-c5ccc(N6CCC(N7CCN(c8ccc(C(=O)O)cc8)CC7)CC6)nc5)ncc43)nn2C2CCOCC2)C1 ; CC(=O)N1CCc2c(c(N3CCCc4cc(-c5ccc(N6CC(N7CCN(c8ccc(C(=O)O)cc8)CC7)C6)nc5)ncc43)nn2C2CCOCC2)C1]

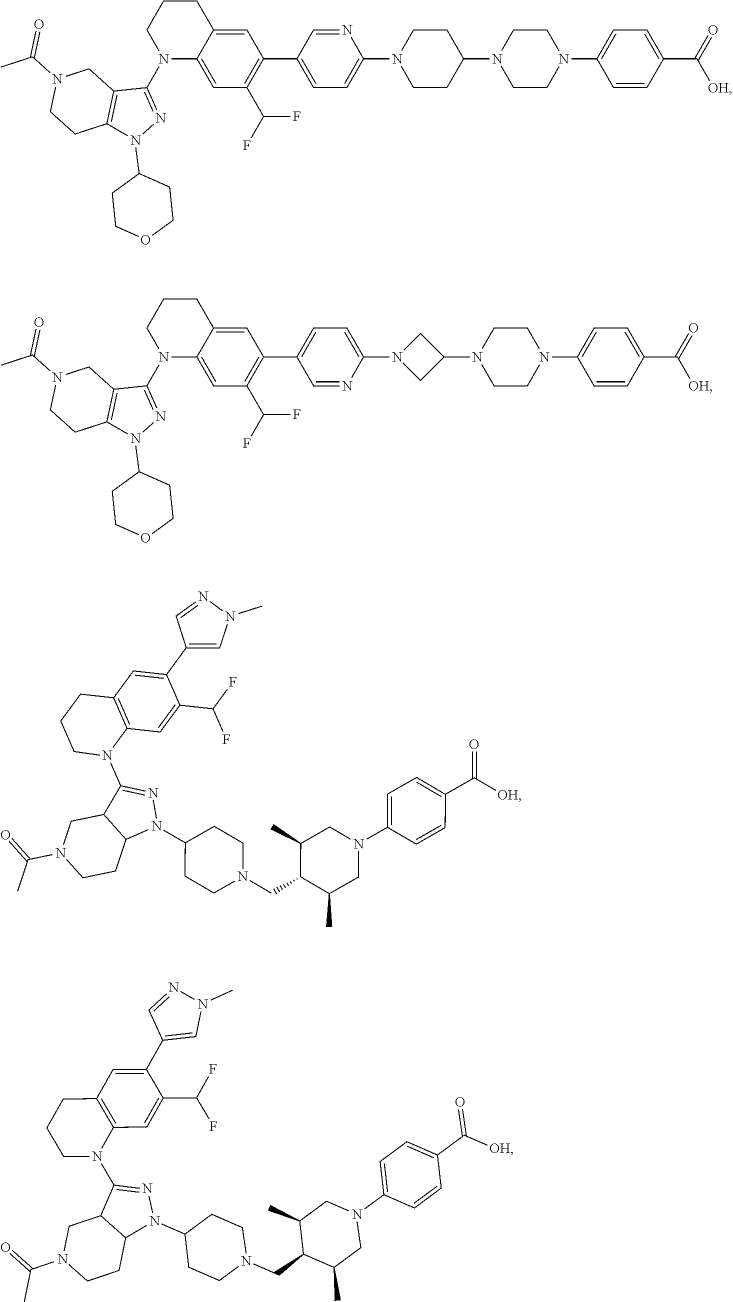 [SMILES: CC(=O)N1CCc2c(c(N3CCCc4cc(-c5ccc(N6CCC(N7CCN(c8ccc(C(=O)O)cc8)CC7)CC6)nc5)c(C(F)F)cc43)nn2C2CCOCC2)C1 ; CC(=O)N1CCc2c(c(N3CCCc4cc(-c5ccc(N6CC(N7CCN(c8ccc(C(=O)O)cc8)CC7)C6)nc5)c(C(F)F)cc43)nn2C2CCOCC2)C1 ; CC(=O)N1CCC2C(C1)C(N1CCCc3cc(-c4cnn(C)c4)c(C(F)F)cc31)=NN2C1CCN(C[C@H]2[C@H](C)CN(c3ccc(C(=O)O)cc3)C[C@@H]2C)CC1 ; CC(=O)N1CCC2C(C1)C(N1CCCc3cc(-c4cnn(C)c4)c(C(F)F)cc31)=NN2C1CCN(C[C@@H]2[C@H](C)CN(c3ccc(C(=O)O)cc3)C[C@@H]2C)CC1]

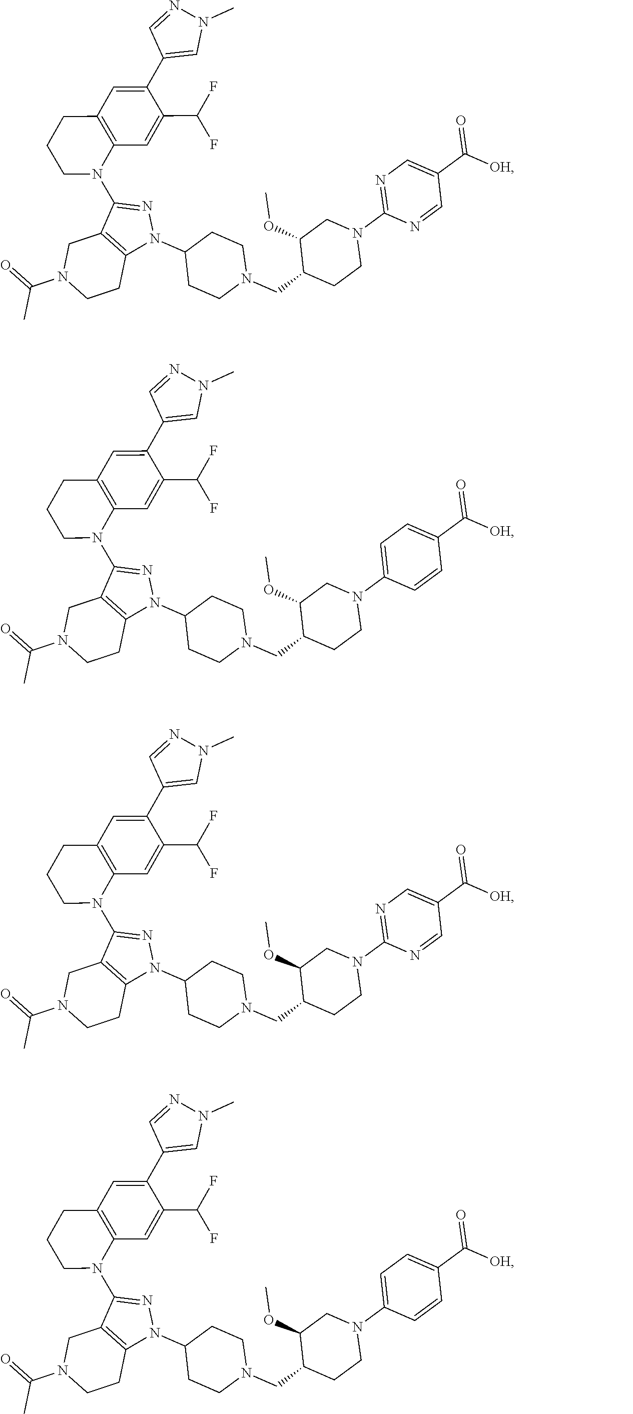 [SMILES: CO[C@@H]1CN(c2ncc(C(=O)O)cn2)CC[C@@H]1CN1CCC(n2nc(N3CCCc4cc(-c5cnn(C)c5)c(C(F)F)cc43)c3c2CCN(C(C)=O)C3)CC1 ; CO[C@@H]1CN(c2ccc(C(=O)O)cc2)CC[C@@H]1CN1CCC(n2nc(N3CCCc4cc(-c5cnn(C)c5)c(C(F)F)cc43)c3c2CCN(C(C)=O)C3)CC1 ; CO[C@H]1CN(c2ncc(C(=O)O)cn2)CC[C@@H]1CN1CCC(n2nc(N3CCCc4cc(-c5cnn(C)c5)c(C(F)F)cc43)c3c2CCN(C(C)=O)C3)CC1 ; CO[C@H]1CN(c2ccc(C(=O)O)cc2)CC[C@@H]1CN1CCC(n2nc(N3CCCc4cc(-c5cnn(C)c5)c(C(F)F)cc43)c3c2CCN(C(C)=O)C3)CC1]

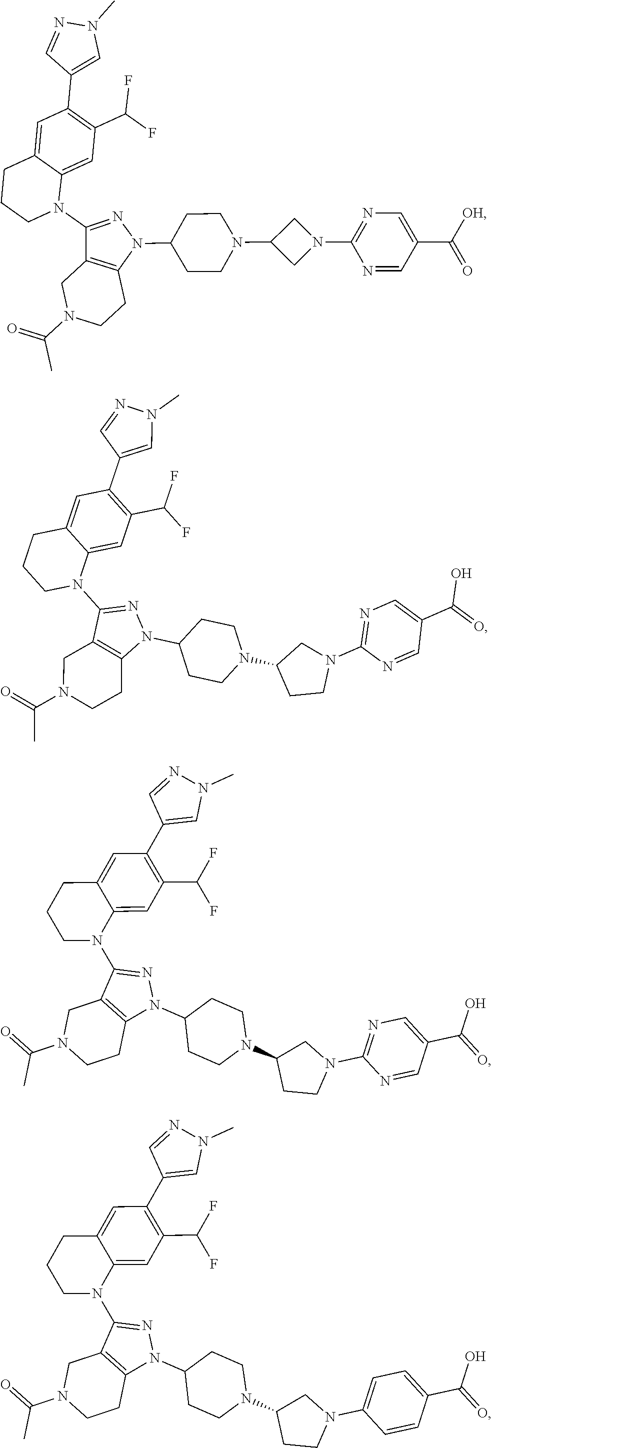 [SMILES: CC(=O)N1CCc2c(c(N3CCCc4cc(-c5cnn(C)c5)c(C(F)F)cc43)nn2C2CCN(C3CN(c4ncc(C(=O)O)cn4)C3)CC2)C1 ; CC(=O)N1CCc2c(c(N3CCCc4cc(-c5cnn(C)c5)c(C(F)F)cc43)nn2C2CCN([C@H]3CCN(c4ncc(C(=O)O)cn4)C3)CC2)C1 ; CC(=O)N1CCc2c(c(N3CCCc4cc(-c5cnn(C)c5)c(C(F)F)cc43)nn2C2CCN([C@@H]3CCN(c4ncc(C(=O)O)cn4)C3)CC2)C1 ; CC(=O)N1CCc2c(c(N3CCCc4cc(-c5cnn(C)c5)c(C(F)F)cc43)nn2C2CCN([C@H]3CCN(c4ccc(C(=O)O)cc4)C3)CC2)C1]

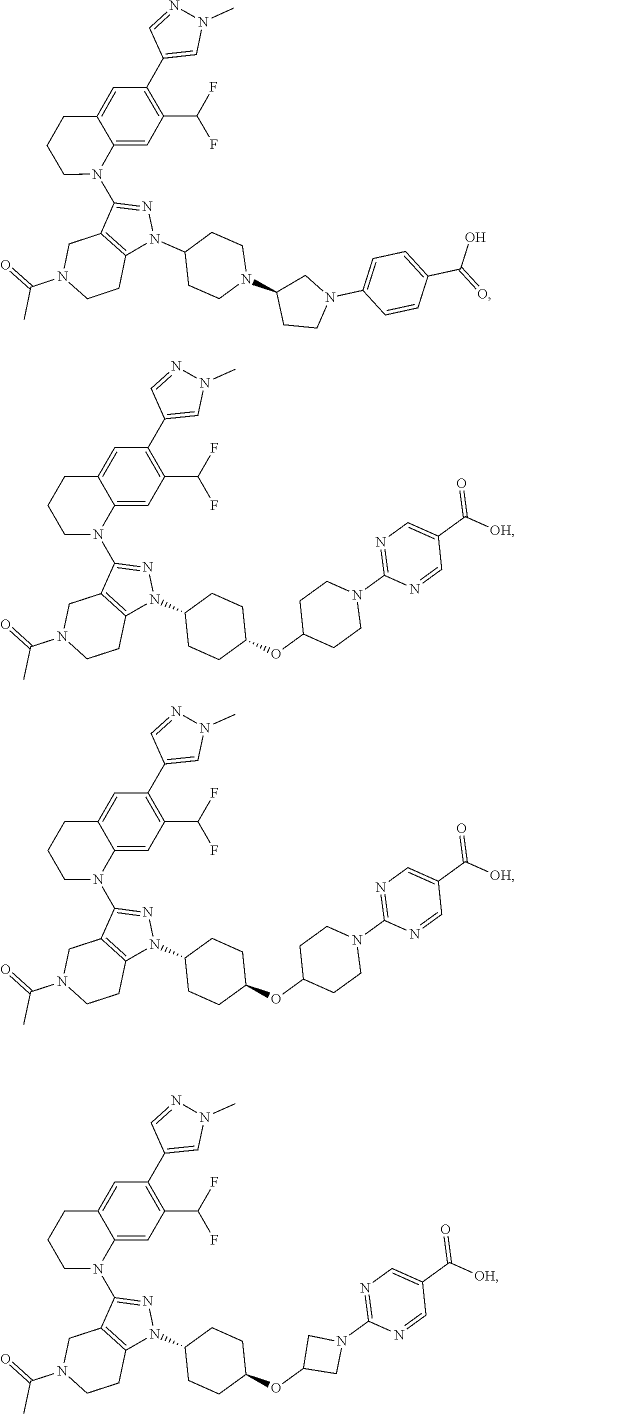 [SMILES: CC(=O)N1CCc2c(c(N3CCCc4cc(-c5cnn(C)c5)c(C(F)F)cc43)nn2C2CCN([C@@H]3CCN(c4ccc(C(=O)O)cc4)C3)CC2)C1 ; CC(=O)N1CCc2c(c(N3CCCc4cc(-c5cnn(C)c5)c(C(F)F)cc43)nn2[C@H]2CC[C@@H](OC3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@H](OC3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@H](OC3CN(c4ncc(C(=O)O)cn4)C3)CC2)C1]

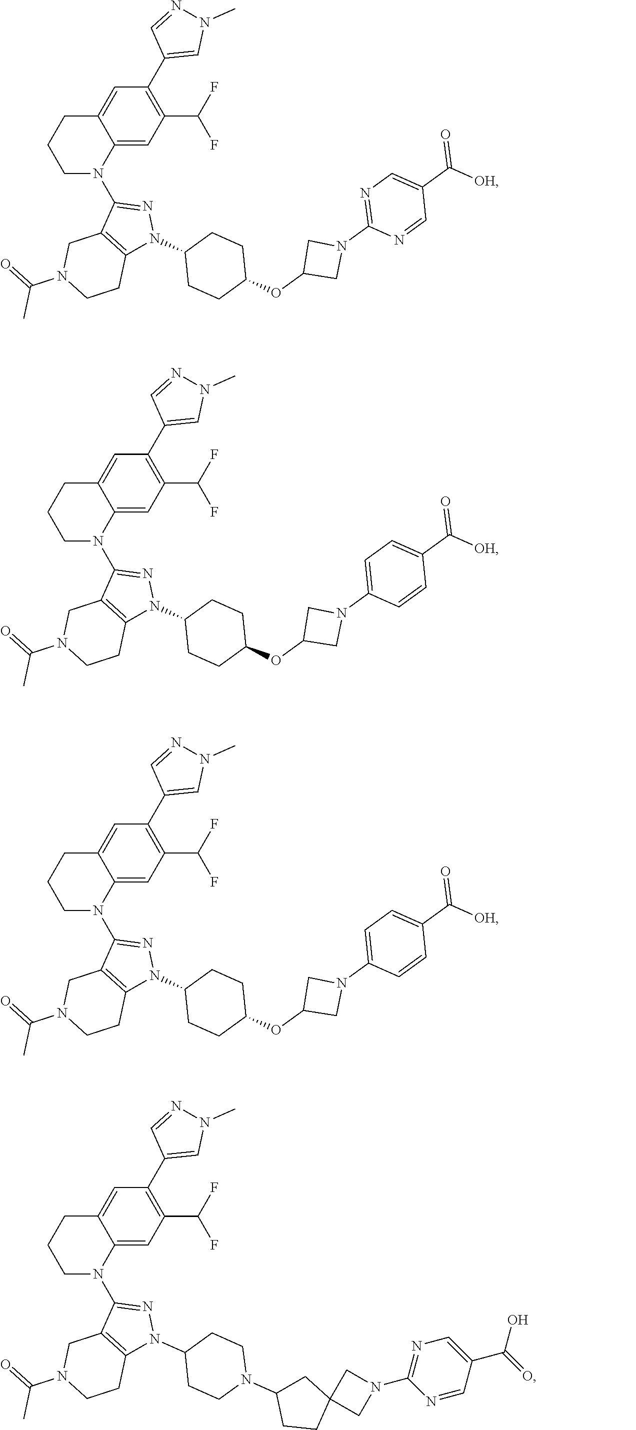 [SMILES: CC(=O)N1CCc2c(c(N3CCCc4cc(-c5cnn(C)c5)c(C(F)F)cc43)nn2[C@H]2CC[C@@H](OC3CN(c4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@H](OC3CN(c4ccc(C(=O)O)cc4)C3)CC2)C1 ; CC(=O)N1CCc2c(c(N3CCCc4cc(-c5cnn(C)c5)c(C(F)F)cc43)nn2[C@H]2CC[C@@H](OC3CN(c4ccc(C(=O)O)cc4)C3)CC2)C1 ; CC(=O)N1CCc2c(c(N3CCCc4cc(-c5cnn(C)c5)c(C(F)F)cc43)nn2C2CCN(C3CCC4(C3)CN(c3ncc(C(=O)O)cn3)C4)CC2)C1]

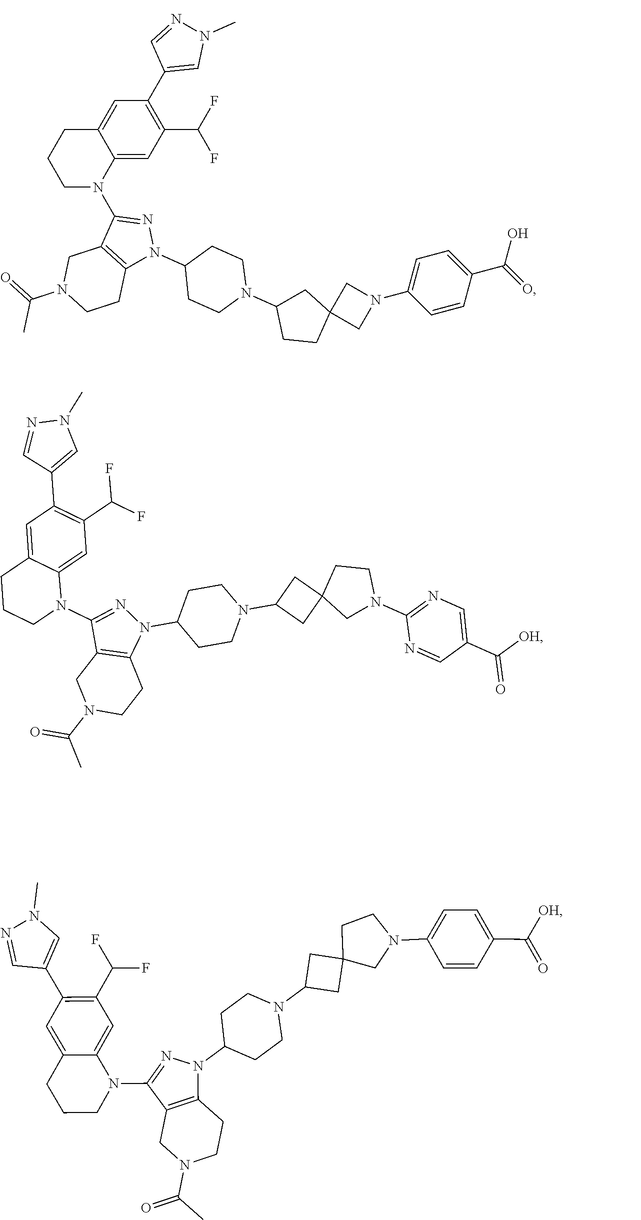 [SMILES: CC(=O)N1CCc2c(c(N3CCCc4cc(-c5cnn(C)c5)c(C(F)F)cc43)nn2C2CCN(C3CCC4(C3)CN(c3ccc(C(=O)O)cc3)C4)CC2)C1 ; CC(=O)N1CCc2c(c(N3CCCc4cc(-c5cnn(C)c5)c(C(F)F)cc43)nn2C2CCN(C3CC4(CCN(c5ncc(C(=O)O)cn5)C4)C3)CC2)C1 ; CC(=O)N1CCc2c(c(N3CCCc4cc(-c5cnn(C)c5)c(C(F)F)cc43)nn2C2CCN(C3CC4(CCN(c5ccc(C(=O)O)cc5)C4)C3)CC2)C1]

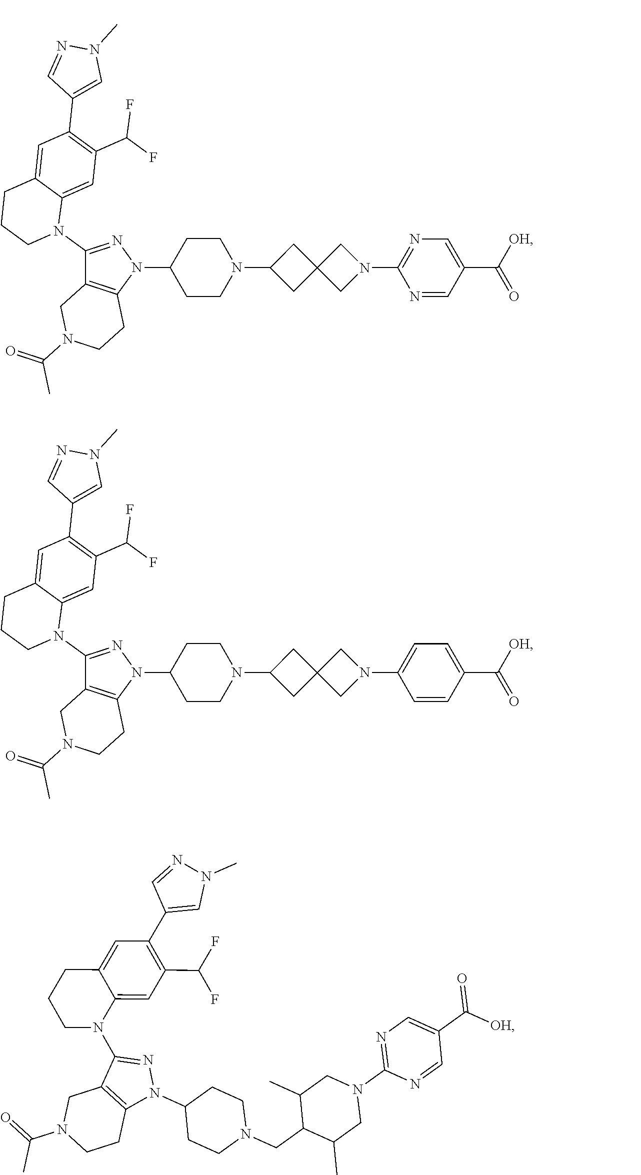 [SMILES: CC(=O)N1CCc2c(c(N3CCCc4cc(-c5cnn(C)c5)c(C(F)F)cc43)nn2C2CCN(C3CC4(C3)CN(c3ncc(C(=O)O)cn3)C4)CC2)C1 ; CC(=O)N1CCc2c(c(N3CCCc4cc(-c5cnn(C)c5)c(C(F)F)cc43)nn2C2CCN(C3CC4(C3)CN(c3ccc(C(=O)O)cc3)C4)CC2)C1 ; CC(=O)N1CCc2c(c(N3CCCc4cc(-c5cnn(C)c5)c(C(F)F)cc43)nn2C2CCN(CC3C(C)CN(c4ncc(C(=O)O)cn4)CC3C)CC2)C1]

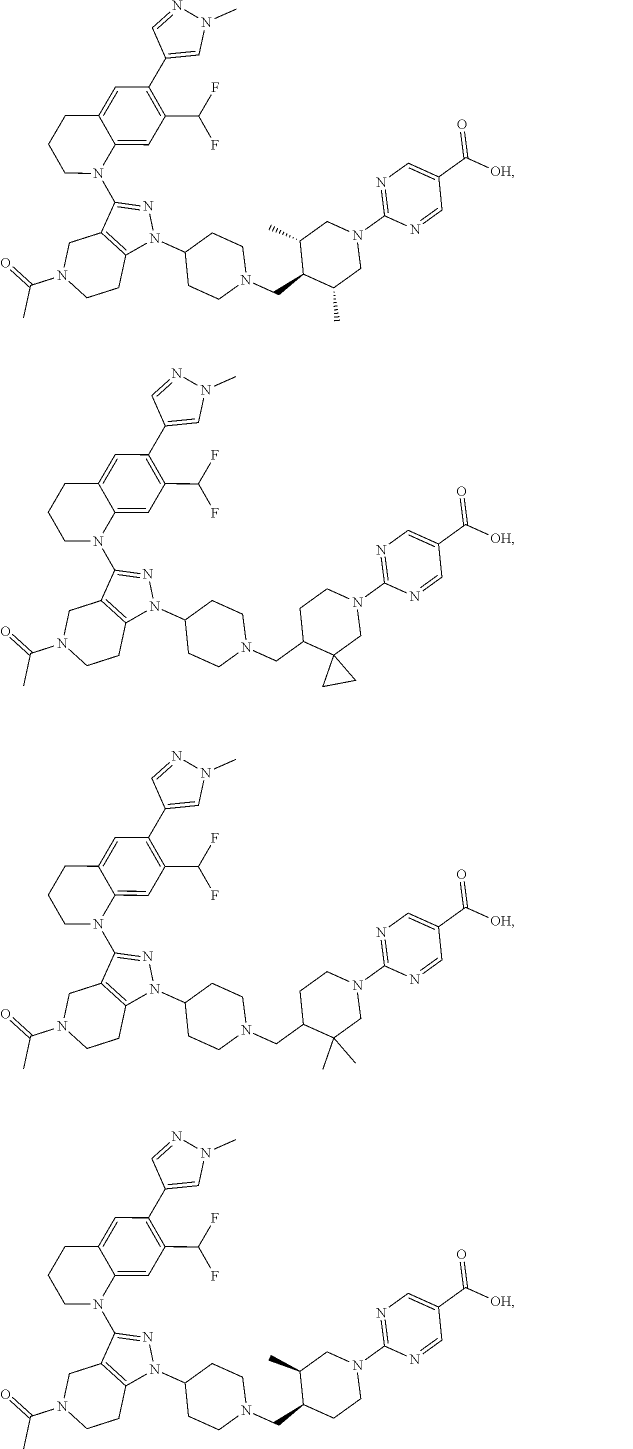 [SMILES: CC(=O)N1CCc2c(c(N3CCCc4cc(-c5cnn(C)c5)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5cnn(C)c5)c(C(F)F)cc43)nn2C2CCN(CC3CCN(c4ncc(C(=O)O)cn4)CC34CC4)CC2)C1 ; CC(=O)N1CCc2c(c(N3CCCc4cc(-c5cnn(C)c5)c(C(F)F)cc43)nn2C2CCN(CC3CCN(c4ncc(C(=O)O)cn4)CC3(C)C)CC2)C1 ; CC(=O)N1CCc2c(c(N3CCCc4cc(-c5cnn(C)c5)c(C(F)F)cc43)nn2C2CCN(C[C@@H]3CCN(c4ncc(C(=O)O)cn4)C[C@@H]3C)CC2)C1]

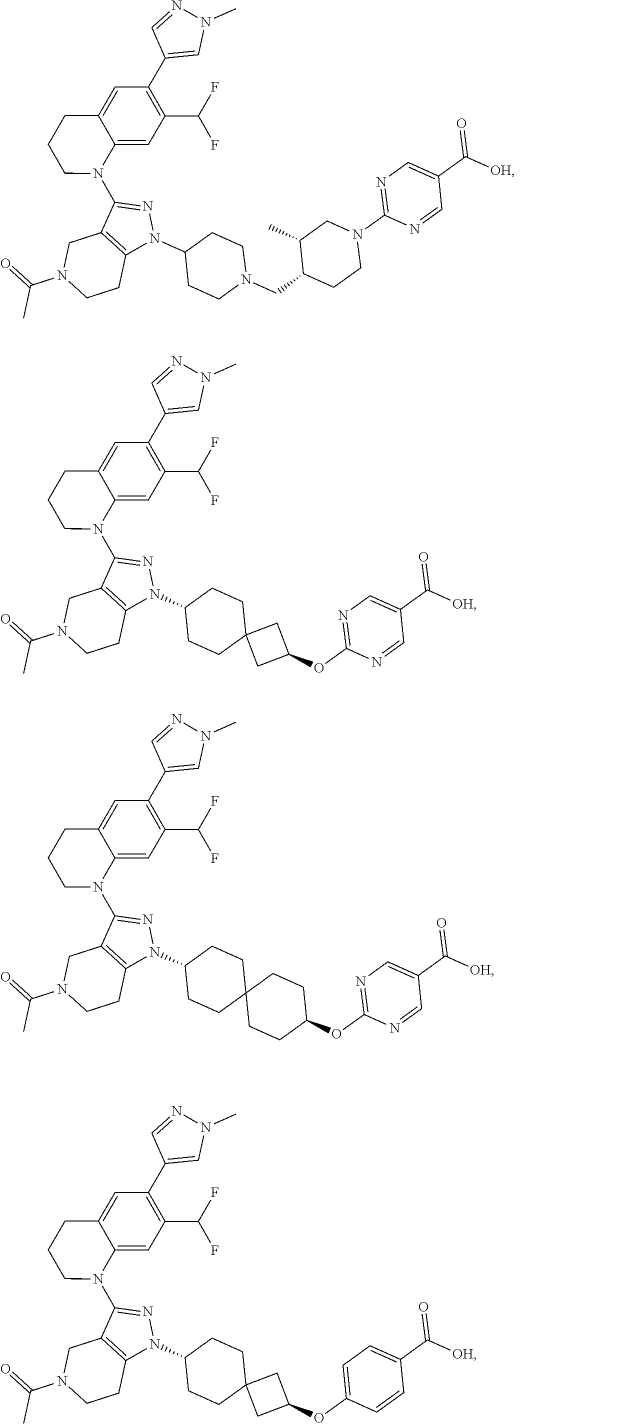 [SMILES: CC(=O)N1CCc2c(c(N3CCCc4cc(-c5cnn(C)c5)c(C(F)F)cc43)nn2C2CCN(C[C@H]3CCN(c4ncc(C(=O)O)cn4)C[C@H]3C)CC2)C1 ; CC(=O)N1CCc2c(c(N3CCCc4cc(-c5cnn(C)c5)c(C(F)F)cc43)nn2[C@H]2CCC3(CC2)C[C@H](Oc2ncc(C(=O)O)cn2)C3)C1 ; CC(=O)N1CCc2c(c(N3CCCc4cc(-c5cnn(C)c5)c(C(F)F)cc43)nn2[C@H]2CCC3(CC[C@H](Oc4ncc(C(=O)O)cn4)CC3)CC2)C1 ; CC(=O)N1CCc2c(c(N3CCCc4cc(-c5cnn(C)c5)c(C(F)F)cc43)nn2[C@H]2CCC3(CC2)C[C@H](Oc2ccc(C(=O)O)cc2)C3)C1]

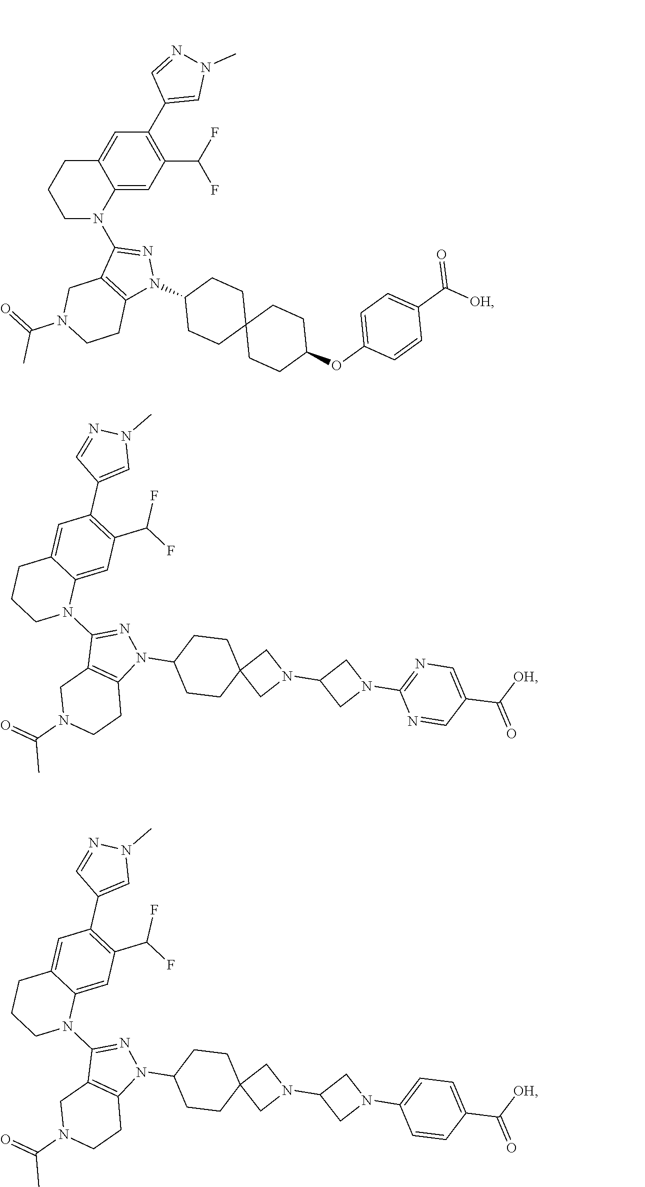 [SMILES: CC(=O)N1CCc2c(c(N3CCCc4cc(-c5cnn(C)c5)c(C(F)F)cc43)nn2[C@H]2CCC3(CC[C@H](Oc4ccc(C(=O)O)cc4)CC3)CC2)C1 ; CC(=O)N1CCc2c(c(N3CCCc4cc(-c5cnn(C)c5)c(C(F)F)cc43)nn2C2CCC3(CC2)CN(C2CN(c4ncc(C(=O)O)cn4)C2)C3)C1 ; CC(=O)N1CCc2c(c(N3CCCc4cc(-c5cnn(C)c5)c(C(F)F)cc43)nn2C2CCC3(CC2)CN(C2CN(c4ccc(C(=O)O)cc4)C2)C3)C1]

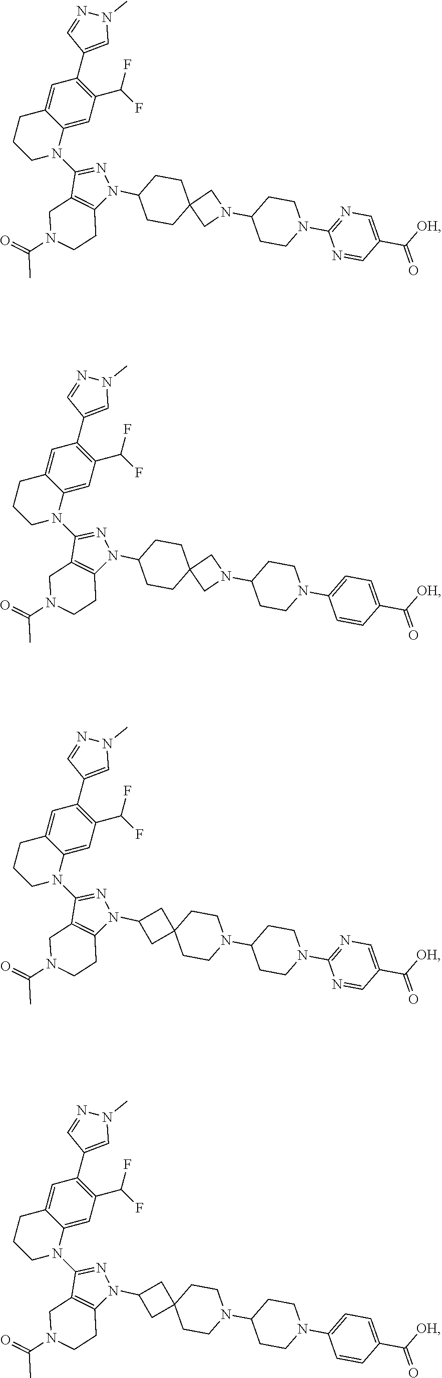 [SMILES: CC(=O)N1CCc2c(c(N3CCCc4cc(-c5cnn(C)c5)c(C(F)F)cc43)nn2C2CCC3(CC2)CN(C2CCN(c4ncc(C(=O)O)cn4)CC2)C3)C1 ; CC(=O)N1CCc2c(c(N3CCCc4cc(-c5cnn(C)c5)c(C(F)F)cc43)nn2C2CCC3(CC2)CN(C2CCN(c4ccc(C(=O)O)cc4)CC2)C3)C1 ; CC(=O)N1CCc2c(c(N3CCCc4cc(-c5cnn(C)c5)c(C(F)F)cc43)nn2C2CC3(CCN(C4CCN(c5ncc(C(=O)O)cn5)CC4)CC3)C2)C1 ; CC(=O)N1CCc2c(c(N3CCCc4cc(-c5cnn(C)c5)c(C(F)F)cc43)nn2C2CC3(CCN(C4CCN(c5ccc(C(=O)O)cc5)CC4)CC3)C2)C1]

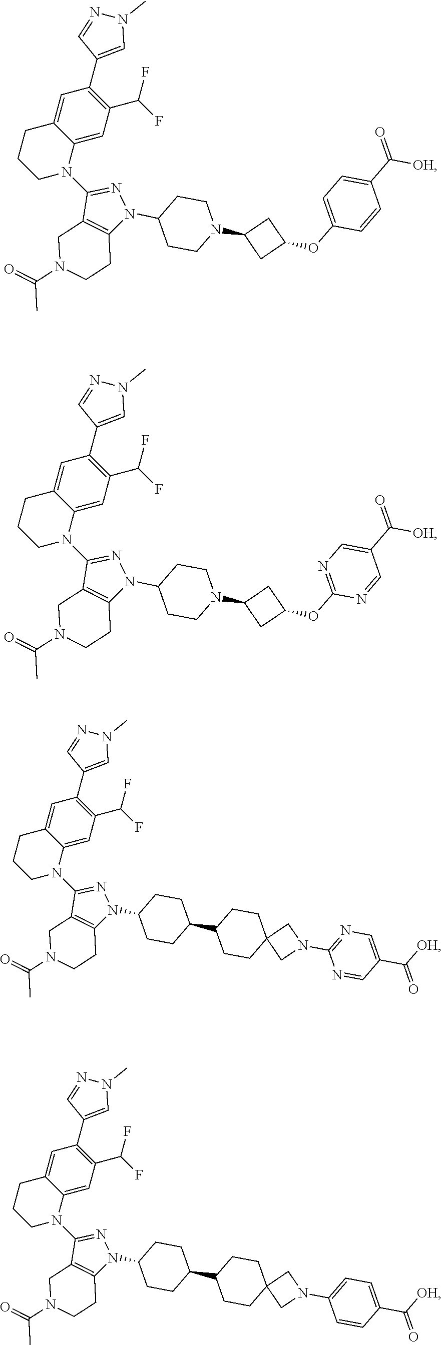 [SMILES: CC(=O)N1CCc2c(c(N3CCCc4cc(-c5cnn(C)c5)c(C(F)F)cc43)nn2C2CCN([C@H]3C[C@H](Oc4ccc(C(=O)O)cc4)C3)CC2)C1 ; CC(=O)N1CCc2c(c(N3CCCc4cc(-c5cnn(C)c5)c(C(F)F)cc43)nn2C2CCN([C@H]3C[C@H](Oc4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@H](C3CCC4(CC3)CN(c3ncc(C(=O)O)cn3)C4)CC2)C1 ; CC(=O)N1CCc2c(c(N3CCCc4cc(-c5cnn(C)c5)c(C(F)F)cc43)nn2[C@H]2CC[C@H](C3CCC4(CC3)CN(c3ccc(C(=O)O)cc3)C4)CC2)C1]

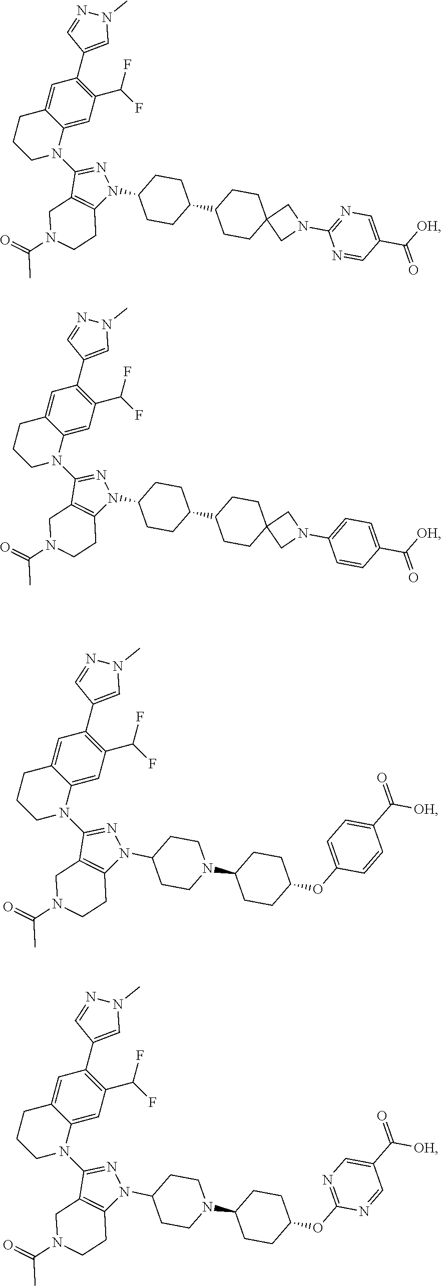 [SMILES: CC(=O)N1CCc2c(c(N3CCCc4cc(-c5cnn(C)c5)c(C(F)F)cc43)nn2[C@H]2CC[C@@H](C3CCC4(CC3)CN(c3ncc(C(=O)O)cn3)C4)CC2)C1 ; CC(=O)N1CCc2c(c(N3CCCc4cc(-c5cnn(C)c5)c(C(F)F)cc43)nn2[C@H]2CC[C@@H](C3CCC4(CC3)CN(c3ccc(C(=O)O)cc3)C4)CC2)C1 ; CC(=O)N1CCc2c(c(N3CCCc4cc(-c5cnn(C)c5)c(C(F)F)cc43)nn2C2CCN([C@H]3CC[C@H](Oc4ccc(C(=O)O)cc4)CC3)CC2)C1 ; CC(=O)N1CCc2c(c(N3CCCc4cc(-c5cnn(C)c5)c(C(F)F)cc43)nn2C2CCN([C@H]3CC[C@H](Oc4ncc(C(=O)O)cn4)CC3)CC2)C1]

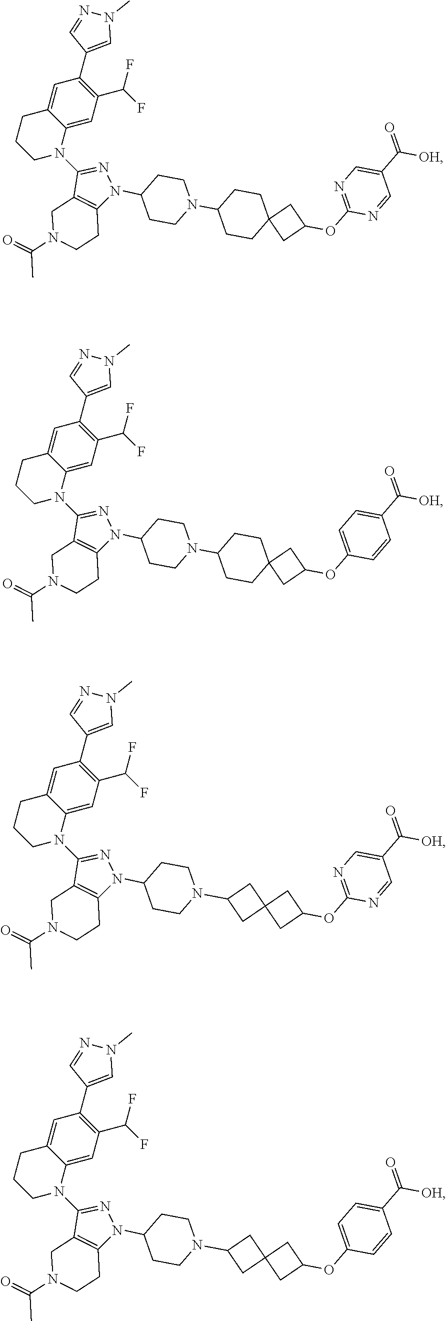 [SMILES: CC(=O)N1CCc2c(c(N3CCCc4cc(-c5cnn(C)c5)c(C(F)F)cc43)nn2C2CCN(C3CCC4(CC3)CC(Oc3ncc(C(=O)O)cn3)C4)CC2)C1 ; CC(=O)N1CCc2c(c(N3CCCc4cc(-c5cnn(C)c5)c(C(F)F)cc43)nn2C2CCN(C3CCC4(CC3)CC(Oc3ccc(C(=O)O)cc3)C4)CC2)C1 ; CC(=O)N1CCc2c(c(N3CCCc4cc(-c5cnn(C)c5)c(C(F)F)cc43)nn2C2CCN(C3CC4(CC(Oc5ncc(C(=O)O)cn5)C4)C3)CC2)C1 ; CC(=O)N1CCc2c(c(N3CCCc4cc(-c5cnn(C)c5)c(C(F)F)cc43)nn2C2CCN(C3CC4(CC(Oc5ccc(C(=O)O)cc5)C4)C3)CC2)C1]

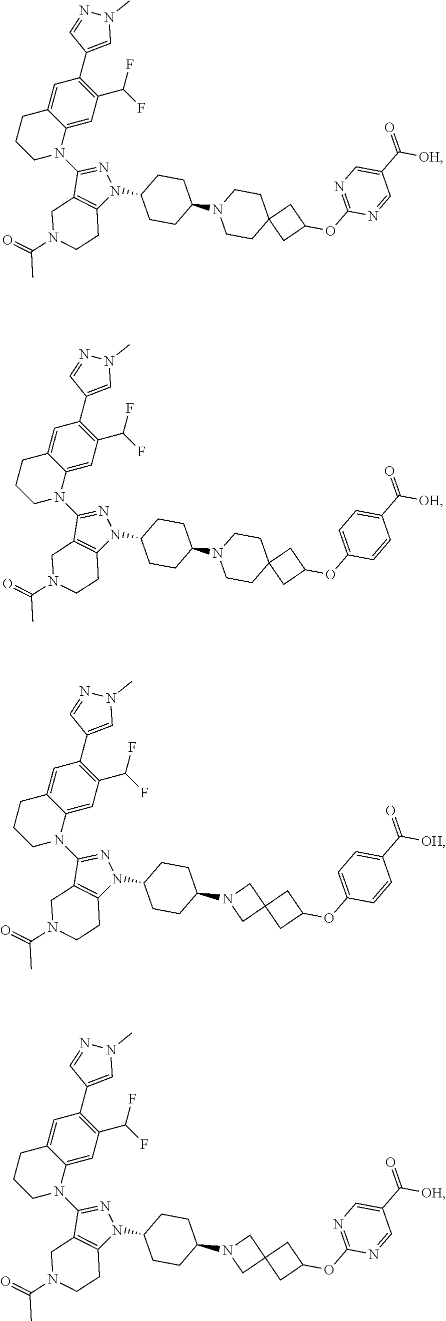 [SMILES: CC(=O)N1CCc2c(c(N3CCCc4cc(-c5cnn(C)c5)c(C(F)F)cc43)nn2[C@H]2CC[C@H](N3CCC4(CC3)CC(Oc3ncc(C(=O)O)cn3)C4)CC2)C1 ; CC(=O)N1CCc2c(c(N3CCCc4cc(-c5cnn(C)c5)c(C(F)F)cc43)nn2[C@H]2CC[C@H](N3CCC4(CC3)CC(Oc3ccc(C(=O)O)cc3)C4)CC2)C1 ; CC(=O)N1CCc2c(c(N3CCCc4cc(-c5cnn(C)c5)c(C(F)F)cc43)nn2[C@H]2CC[C@H](N3CC4(CC(Oc5ccc(C(=O)O)cc5)C4)C3)CC2)C1 ; CC(=O)N1CCc2c(c(N3CCCc4cc(-c5cnn(C)c5)c(C(F)F)cc43)nn2[C@H]2CC[C@H](N3CC4(CC(Oc5ncc(C(=O)O)cn5)C4)C3)CC2)C1]

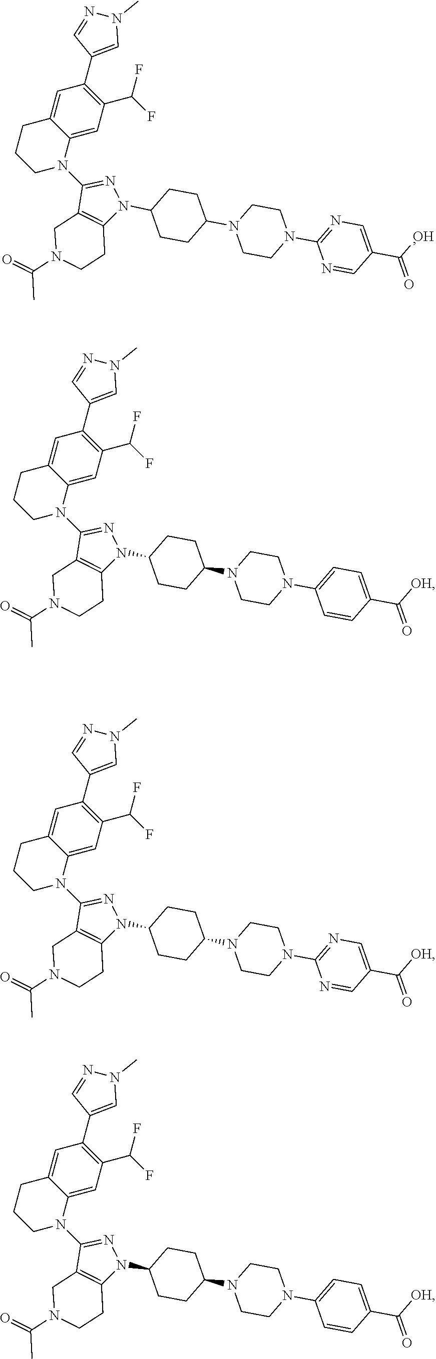 [SMILES: CC(=O)N1CCc2c(c(N3CCCc4cc(-c5cnn(C)c5)c(C(F)F)cc43)nn2C2CCC(N3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@H](N3CCN(c4ccc(C(=O)O)cc4)CC3)CC2)C1 ; CC(=O)N1CCc2c(c(N3CCCc4cc(-c5cnn(C)c5)c(C(F)F)cc43)nn2[C@H]2CC[C@@H](N3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@@H](N3CCN(c4ccc(C(=O)O)cc4)CC3)CC2)C1]

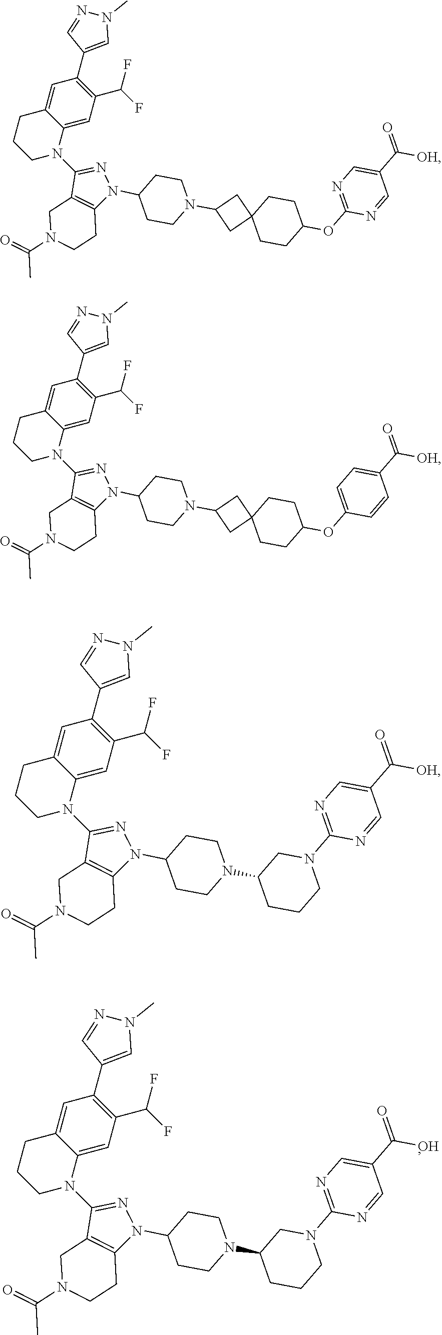 [SMILES: CC(=O)N1CCc2c(c(N3CCCc4cc(-c5cnn(C)c5)c(C(F)F)cc43)nn2C2CCN(C3CC4(CCC(Oc5ncc(C(=O)O)cn5)CC4)C3)CC2)C1 ; CC(=O)N1CCc2c(c(N3CCCc4cc(-c5cnn(C)c5)c(C(F)F)cc43)nn2C2CCN(C3CC4(CCC(Oc5ccc(C(=O)O)cc5)CC4)C3)CC2)C1 ; CC(=O)N1CCc2c(c(N3CCCc4cc(-c5cnn(C)c5)c(C(F)F)cc43)nn2C2CCN([C@H]3CCCN(c4ncc(C(=O)O)cn4)C3)CC2)C1 ; CC(=O)N1CCc2c(c(N3CCCc4cc(-c5cnn(C)c5)c(C(F)F)cc43)nn2C2CCN([C@@H]3CCCN(c4ncc(C(=O)O)cn4)C3)CC2)C1]

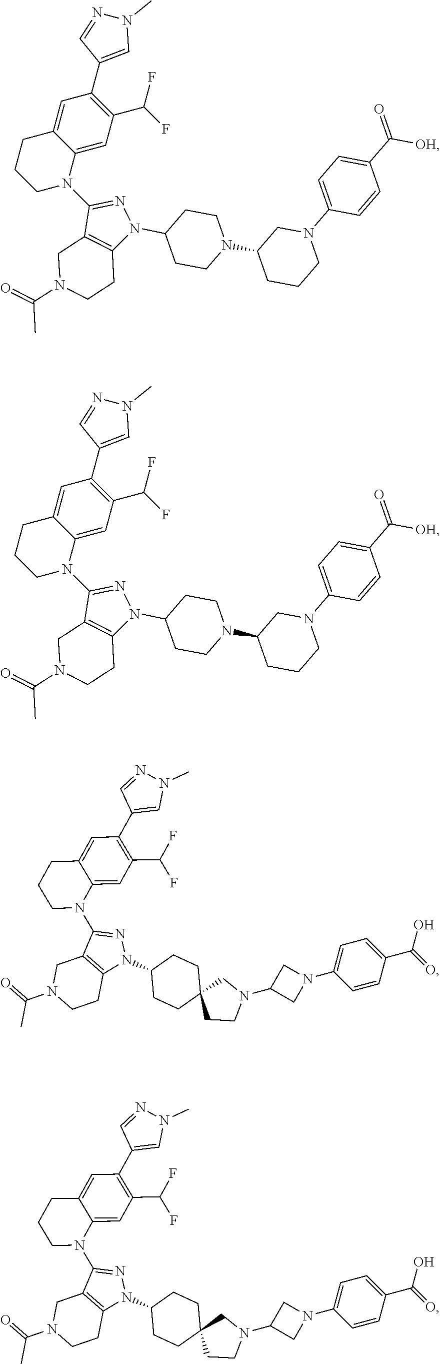 [SMILES: CC(=O)N1CCc2c(c(N3CCCc4cc(-c5cnn(C)c5)c(C(F)F)cc43)nn2C2CCN([C@H]3CCCN(c4ccc(C(=O)O)cc4)C3)CC2)C1 ; CC(=O)N1CCc2c(c(N3CCCc4cc(-c5cnn(C)c5)c(C(F)F)cc43)nn2C2CCN([C@@H]3CCCN(c4ccc(C(=O)O)cc4)C3)CC2)C1 ; CC(=O)N1CCc2c(c(N3CCCc4cc(-c5cnn(C)c5)c(C(F)F)cc43)nn2[C@H]2CC[C@]3(CCN(C4CN(c5ccc(C(=O)O)cc5)C4)C3)CC2)C1 ; CC(=O)N1CCc2c(c(N3CCCc4cc(-c5cnn(C)c5)c(C(F)F)cc43)nn2[C@H]2CC[C@@]3(CCN(C4CN(c5ccc(C(=O)O)cc5)C4)C3)CC2)C1]

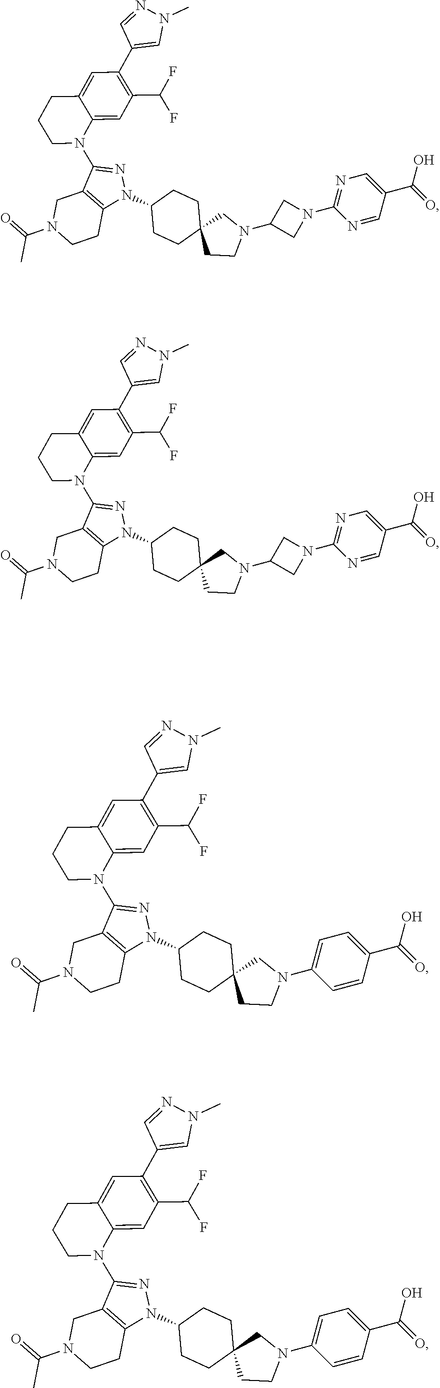 [SMILES: CC(=O)N1CCc2c(c(N3CCCc4cc(-c5cnn(C)c5)c(C(F)F)cc43)nn2[C@H]2CC[C@]3(CCN(C4CN(c5ncc(C(=O)O)cn5)C4)C3)CC2)C1 ; CC(=O)N1CCc2c(c(N3CCCc4cc(-c5cnn(C)c5)c(C(F)F)cc43)nn2[C@H]2CC[C@@]3(CCN(C4CN(c5ncc(C(=O)O)cn5)C4)C3)CC2)C1 ; CC(=O)N1CCc2c(c(N3CCCc4cc(-c5cnn(C)c5)c(C(F)F)cc43)nn2[C@H]2CC[C@]3(CCN(c4ccc(C(=O)O)cc4)C3)CC2)C1 ; CC(=O)N1CCc2c(c(N3CCCc4cc(-c5cnn(C)c5)c(C(F)F)cc43)nn2[C@H]2CC[C@@]3(CCN(c4ccc(C(=O)O)cc4)C3)CC2)C1]

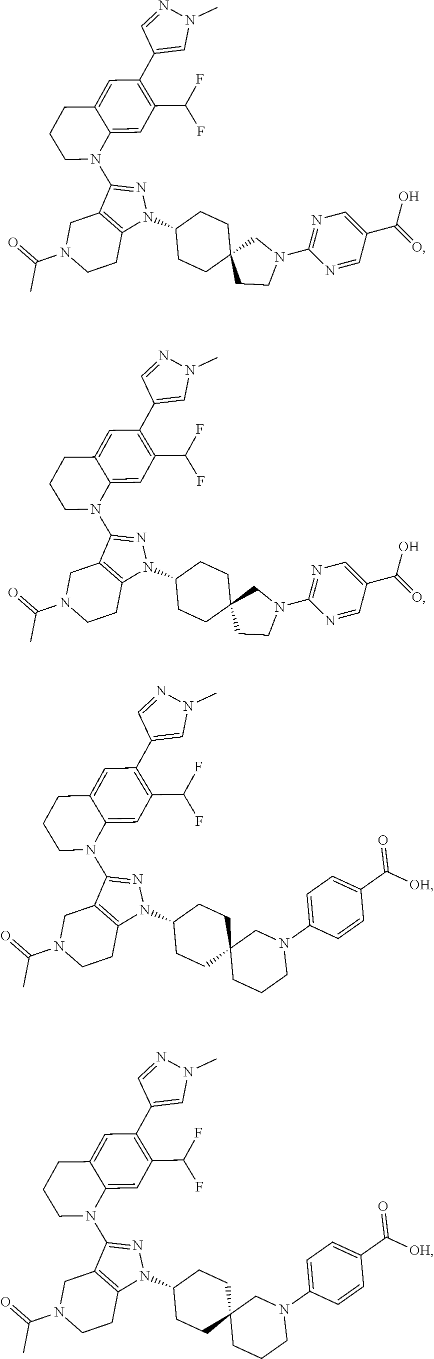 [SMILES: CC(=O)N1CCc2c(c(N3CCCc4cc(-c5cnn(C)c5)c(C(F)F)cc43)nn2[C@H]2CC[C@]3(CCN(c4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@@]3(CCN(c4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@]3(CCCN(c4ccc(C(=O)O)cc4)C3)CC2)C1 ; CC(=O)N1CCc2c(c(N3CCCc4cc(-c5cnn(C)c5)c(C(F)F)cc43)nn2[C@H]2CC[C@@]3(CCCN(c4ccc(C(=O)O)cc4)C3)CC2)C1]

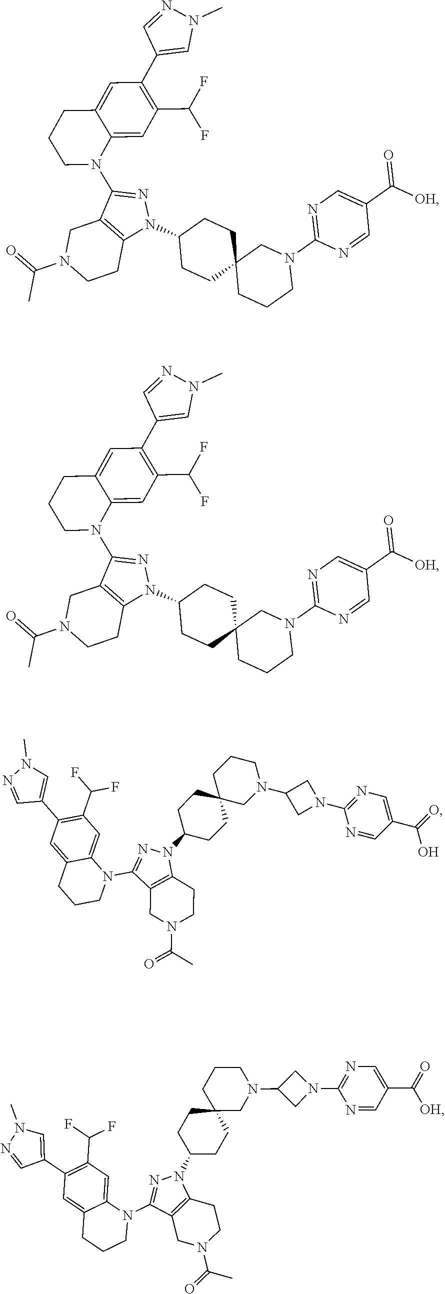 [SMILES: CC(=O)N1CCc2c(c(N3CCCc4cc(-c5cnn(C)c5)c(C(F)F)cc43)nn2[C@H]2CC[C@]3(CCCN(c4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@@]3(CCCN(c4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@]3(CCCN(C4CN(c5ncc(C(=O)O)cn5)C4)C3)CC2)C1 ; CC(=O)N1CCc2c(c(N3CCCc4cc(-c5cnn(C)c5)c(C(F)F)cc43)nn2[C@H]2CC[C@@]3(CCCN(C4CN(c5ncc(C(=O)O)cn5)C4)C3)CC2)C1]

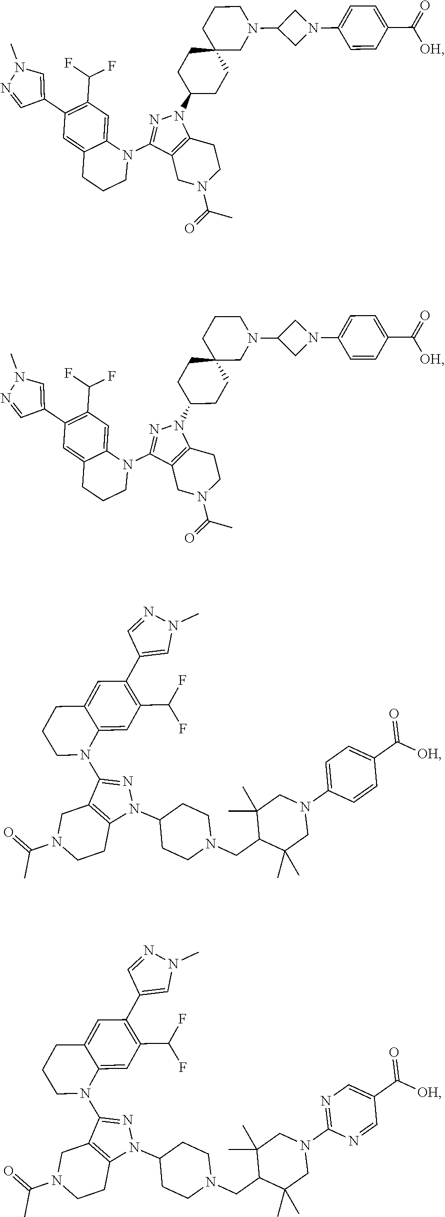 [SMILES: CC(=O)N1CCc2c(c(N3CCCc4cc(-c5cnn(C)c5)c(C(F)F)cc43)nn2[C@H]2CC[C@]3(CCCN(C4CN(c5ccc(C(=O)O)cc5)C4)C3)CC2)C1 ; CC(=O)N1CCc2c(c(N3CCCc4cc(-c5cnn(C)c5)c(C(F)F)cc43)nn2[C@H]2CC[C@@]3(CCCN(C4CN(c5ccc(C(=O)O)cc5)C4)C3)CC2)C1 ; CC(=O)N1CCc2c(c(N3CCCc4cc(-c5cnn(C)c5)c(C(F)F)cc43)nn2C2CCN(CC3C(C)(C)CN(c4ccc(C(=O)O)cc4)CC3(C)C)CC2)C1 ; CC(=O)N1CCc2c(c(N3CCCc4cc(-c5cnn(C)c5)c(C(F)F)cc43)nn2C2CCN(CC3C(C)(C)CN(c4ncc(C(=O)O)cn4)CC3(C)C)CC2)C1]

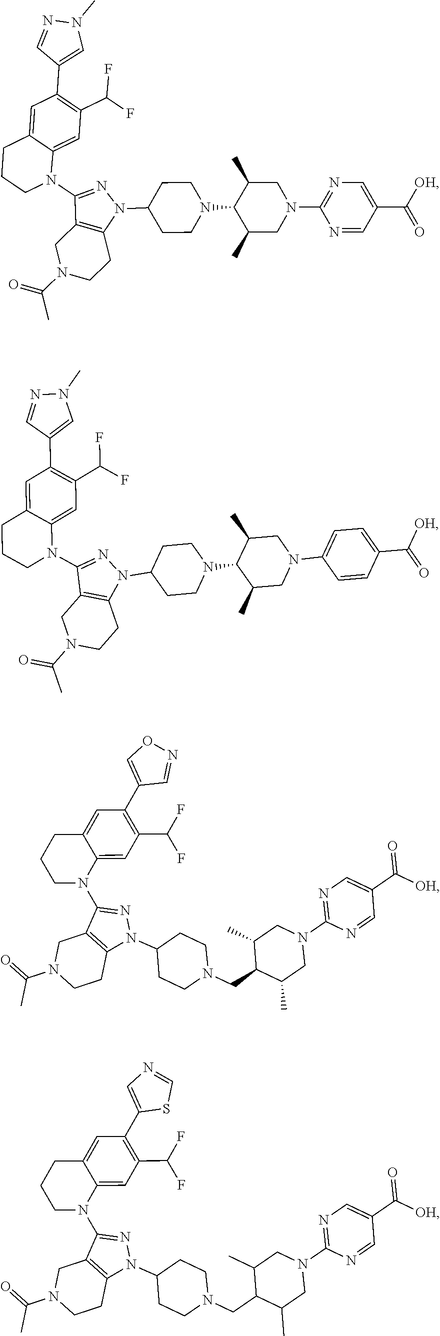 [SMILES: CC(=O)N1CCc2c(c(N3CCCc4cc(-c5cnn(C)c5)c(C(F)F)cc43)nn2C2CCN([C@H]3[C@H](C)CN(c4ncc(C(=O)O)cn4)C[C@@H]3C)CC2)C1 ; CC(=O)N1CCc2c(c(N3CCCc4cc(-c5cnn(C)c5)c(C(F)F)cc43)nn2C2CCN([C@H]3[C@H](C)CN(c4ccc(C(=O)O)cc4)C[C@@H]3C)CC2)C1 ; CC(=O)N1CCc2c(c(N3CCCc4cc(-c5cnoc5)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5cncs5)c(C(F)F)cc43)nn2C2CCN(CC3C(C)CN(c4ncc(C(=O)O)cn4)CC3C)CC2)C1]

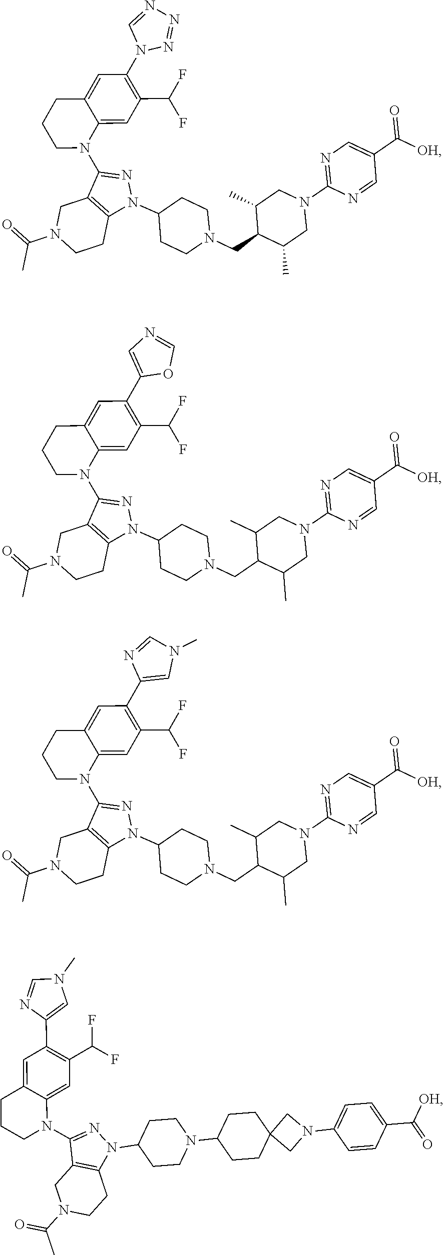 [SMILES: CC(=O)N1CCc2c(c(N3CCCc4cc(-n5cnnn5)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5cnco5)c(C(F)F)cc43)nn2C2CCN(CC3C(C)CN(c4ncc(C(=O)O)cn4)CC3C)CC2)C1 ; CC(=O)N1CCc2c(c(N3CCCc4cc(-c5cn(C)cn5)c(C(F)F)cc43)nn2C2CCN(CC3C(C)CN(c4ncc(C(=O)O)cn4)CC3C)CC2)C1 ; CC(=O)N1CCc2c(c(N3CCCc4cc(-c5cn(C)cn5)c(C(F)F)cc43)nn2C2CCN(C3CCC4(CC3)CN(c3ccc(C(=O)O)cc3)C4)CC2)C1]

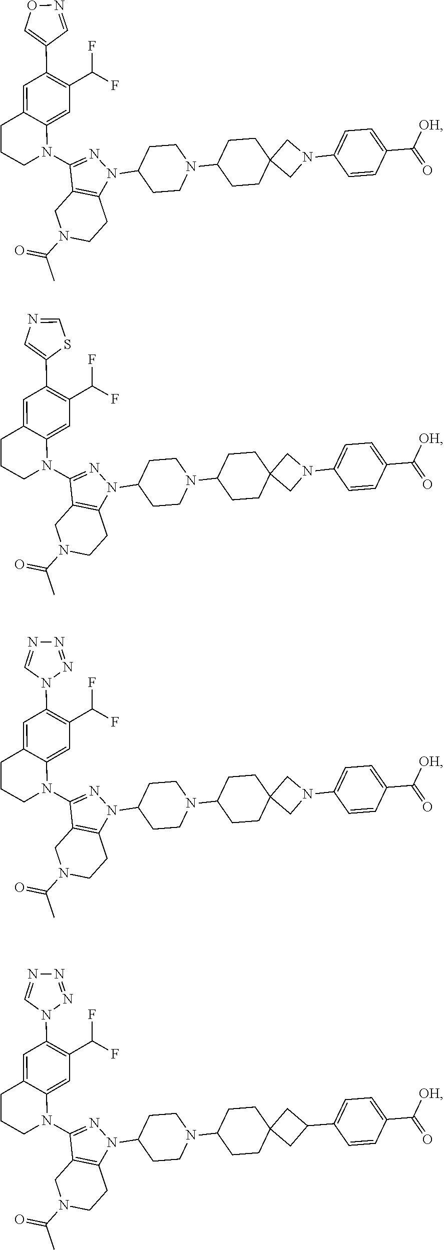 [SMILES: CC(=O)N1CCc2c(c(N3CCCc4cc(-c5cnoc5)c(C(F)F)cc43)nn2C2CCN(C3CCC4(CC3)CN(c3ccc(C(=O)O)cc3)C4)CC2)C1 ; CC(=O)N1CCc2c(c(N3CCCc4cc(-c5cncs5)c(C(F)F)cc43)nn2C2CCN(C3CCC4(CC3)CN(c3ccc(C(=O)O)cc3)C4)CC2)C1 ; CC(=O)N1CCc2c(c(N3CCCc4cc(-n5cnnn5)c(C(F)F)cc43)nn2C2CCN(C3CCC4(CC3)CN(c3ccc(C(=O)O)cc3)C4)CC2)C1 ; CC(=O)N1CCc2c(c(N3CCCc4cc(-n5cnnn5)c(C(F)F)cc43)nn2C2CCN(C3CCC4(CC3)CC(c3ccc(C(=O)O)cc3)C4)CC2)C1]

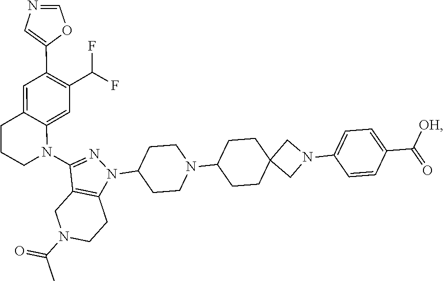 [SMILES: CC(=O)N1CCc2c(c(N3CCCc4cc(-c5cnco5)c(C(F)F)cc43)nn2C2CCN(C3CCC4(CC3)CN(c3ccc(C(=O)O)cc3)C4)CC2)C1]

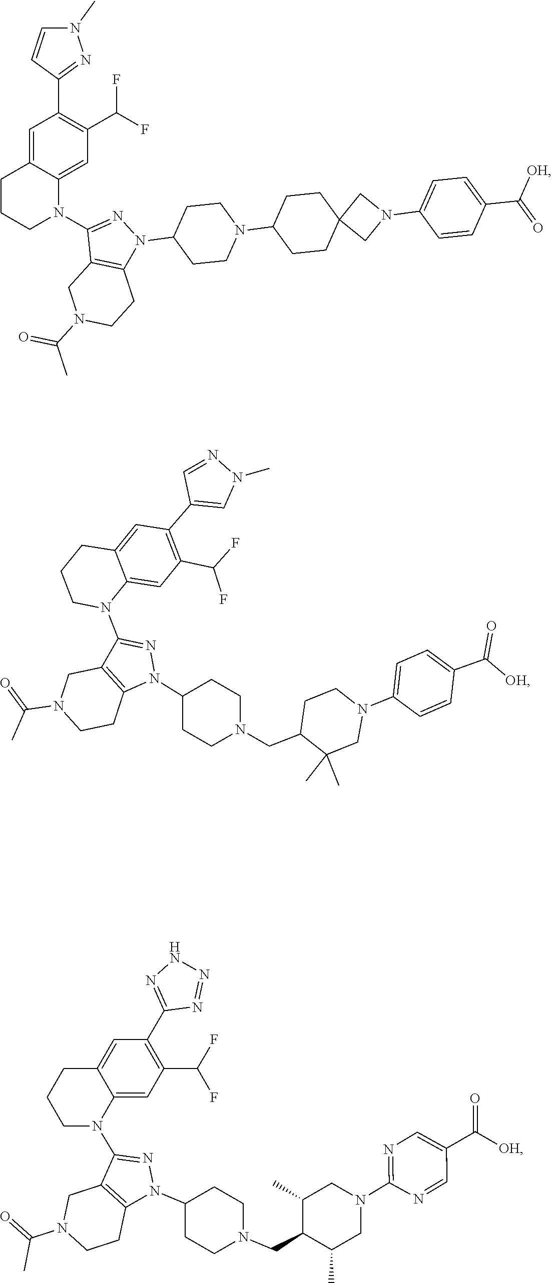 [SMILES: CC(=O)N1CCc2c(c(N3CCCc4cc(-c5ccn(C)n5)c(C(F)F)cc43)nn2C2CCN(C3CCC4(CC3)CN(c3ccc(C(=O)O)cc3)C4)CC2)C1 ; CC(=O)N1CCc2c(c(N3CCCc4cc(-c5cnn(C)c5)c(C(F)F)cc43)nn2C2CCN(CC3CCN(c4ccc(C(=O)O)cc4)CC3(C)C)CC2)C1 ; CC(=O)N1CCc2c(c(N3CCCc4cc(-c5nn[nH]n5)c(C(F)F)cc43)nn2C2CCN(C[C@H]3[C@H](C)CN(c4ncc(C(=O)O)cn4)C[C@@H]3C)CC2)C1]

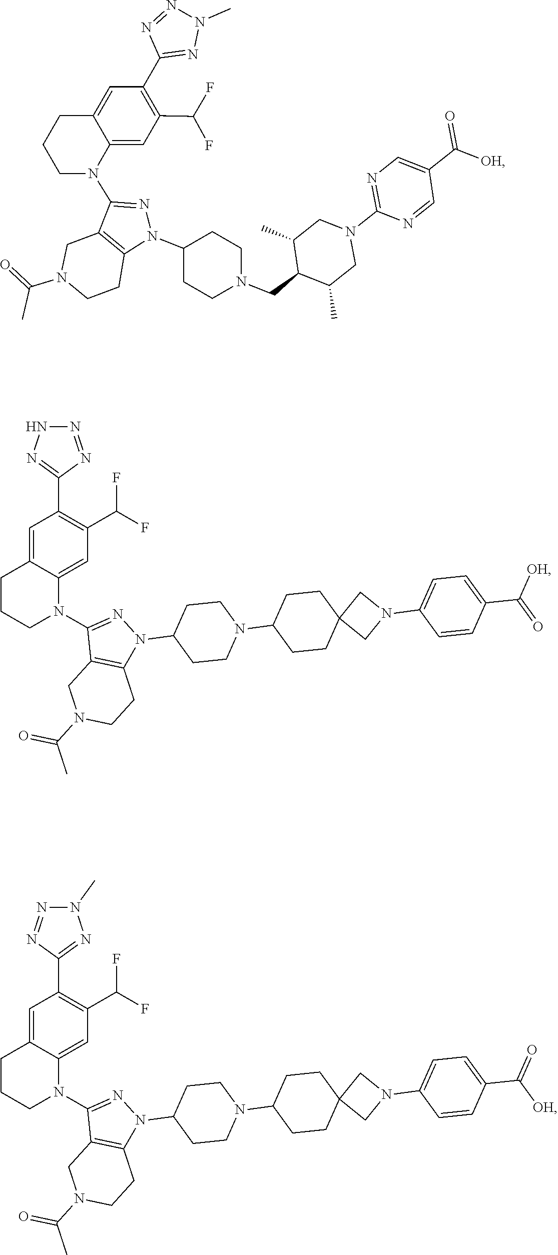 [SMILES: CC(=O)N1CCc2c(c(N3CCCc4cc(-c5nnn(C)n5)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5nn[nH]n5)c(C(F)F)cc43)nn2C2CCN(C3CCC4(CC3)CN(c3ccc(C(=O)O)cc3)C4)CC2)C1 ; CC(=O)N1CCc2c(c(N3CCCc4cc(-c5nnn(C)n5)c(C(F)F)cc43)nn2C2CCN(C3CCC4(CC3)CN(c3ccc(C(=O)O)cc3)C4)CC2)C1]

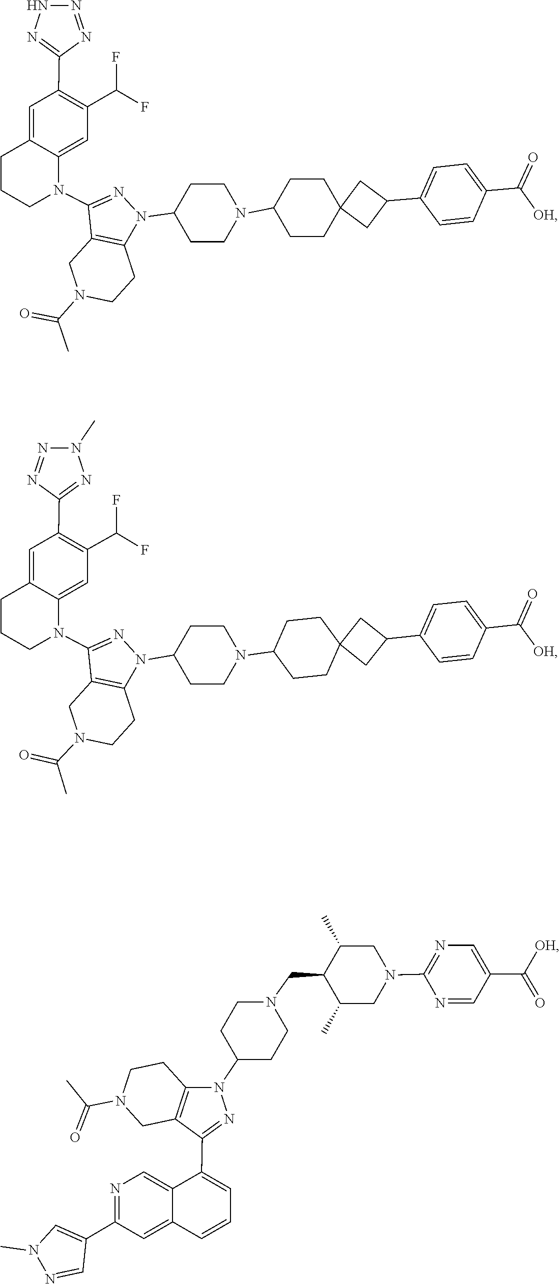 [SMILES: CC(=O)N1CCc2c(c(N3CCCc4cc(-c5nn[nH]n5)c(C(F)F)cc43)nn2C2CCN(C3CCC4(CC3)CC(c3ccc(C(=O)O)cc3)C4)CC2)C1 ; CC(=O)N1CCc2c(c(N3CCCc4cc(-c5nnn(C)n5)c(C(F)F)cc43)nn2C2CCN(C3CCC4(CC3)CC(c3ccc(C(=O)O)cc3)C4)CC2)C1 ; CC(=O)N1CCc2c(c(-c3cccc4cc(-c5cnn(C)c5)ncc34)nn2C2CCN(C[C@H]3[C@H](C)CN(c4ncc(C(=O)O)cn4)C[C@@H]3C)CC2)C1]

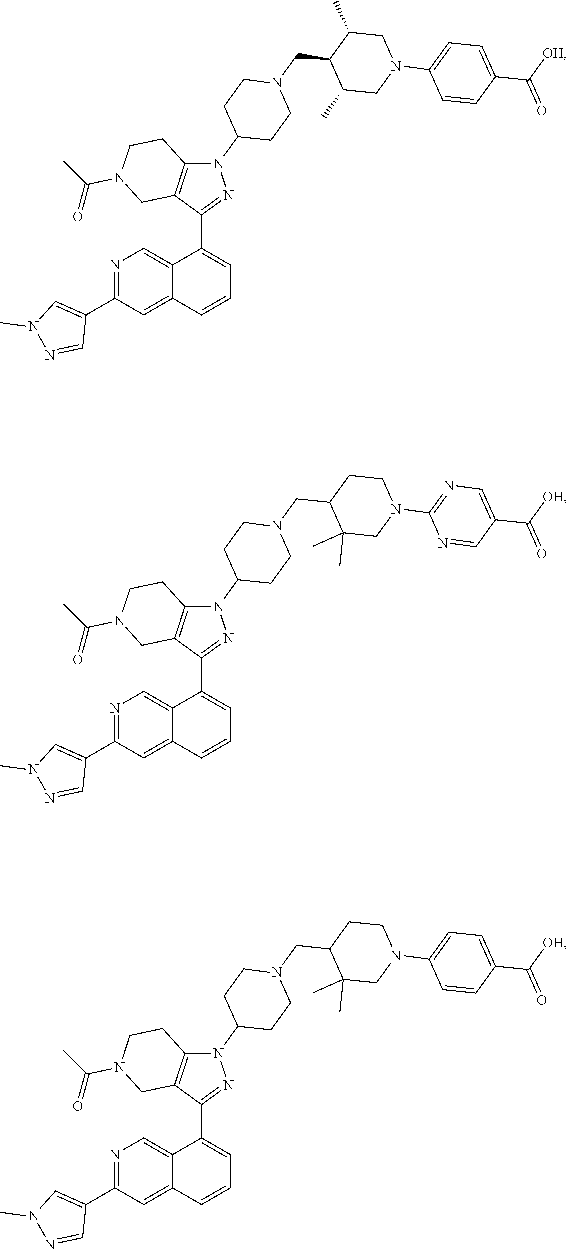 [SMILES: CC(=O)N1CCc2c(c(-c3cccc4cc(-c5cnn(C)c5)ncc34)nn2C2CCN(C[C@H]3[C@H](C)CN(c4ccc(C(=O)O)cc4)C[C@@H]3C)CC2)C1 ; CC(=O)N1CCc2c(c(-c3cccc4cc(-c5cnn(C)c5)ncc34)nn2C2CCN(CC3CCN(c4ncc(C(=O)O)cn4)CC3(C)C)CC2)C1 ; CC(=O)N1CCc2c(c(-c3cccc4cc(-c5cnn(C)c5)ncc34)nn2C2CCN(CC3CCN(c4ccc(C(=O)O)cc4)CC3(C)C)CC2)C1]

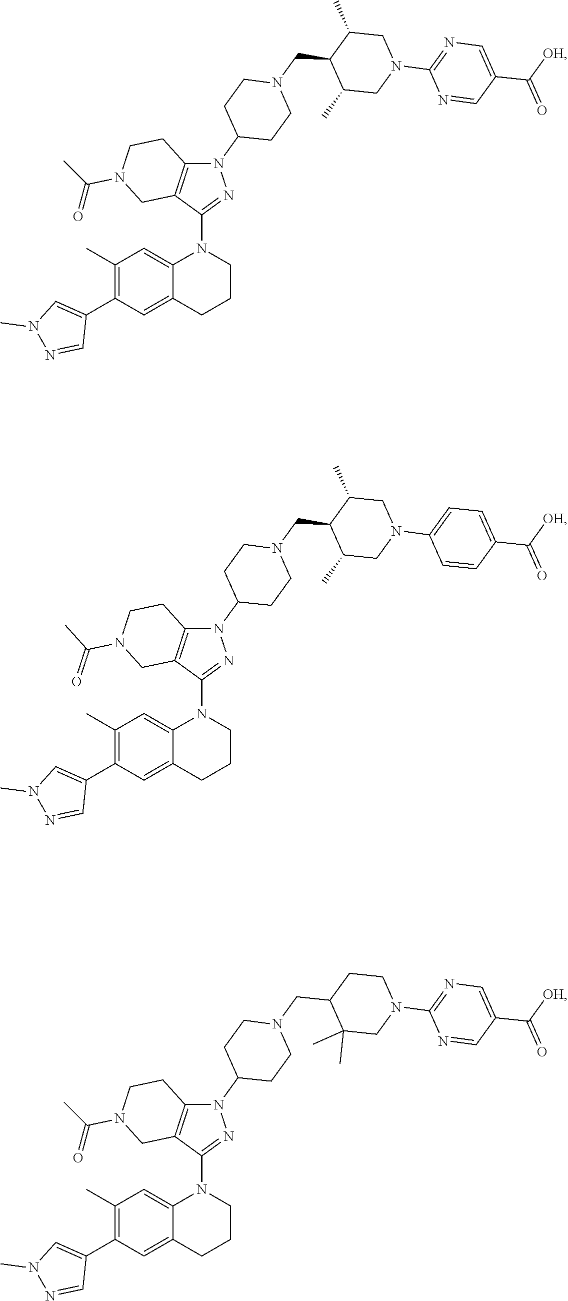 [SMILES: CC(=O)N1CCc2c(c(N3CCCc4cc(-c5cnn(C)c5)c(C)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5cnn(C)c5)c(C)cc43)nn2C2CCN(C[C@H]3[C@H](C)CN(c4ccc(C(=O)O)cc4)C[C@@H]3C)CC2)C1 ; CC(=O)N1CCc2c(c(N3CCCc4cc(-c5cnn(C)c5)c(C)cc43)nn2C2CCN(CC3CCN(c4ncc(C(=O)O)cn4)CC3(C)C)CC2)C1]

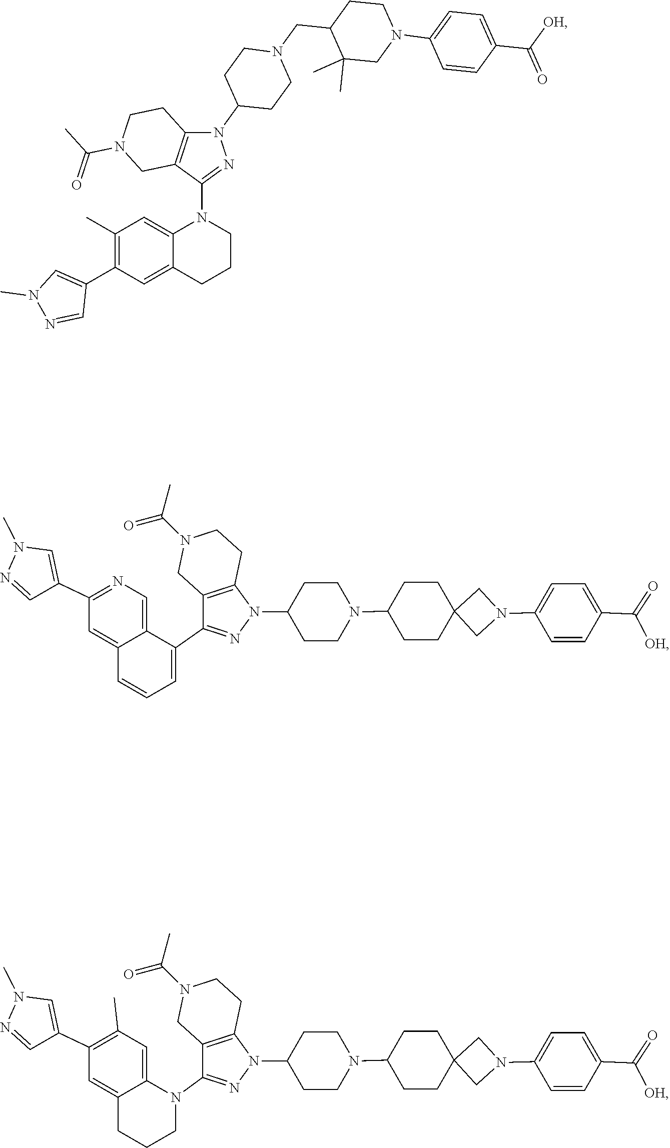 [SMILES: CC(=O)N1CCc2c(c(N3CCCc4cc(-c5cnn(C)c5)c(C)cc43)nn2C2CCN(CC3CCN(c4ccc(C(=O)O)cc4)CC3(C)C)CC2)C1 ; CC(=O)N1CCc2c(c(-c3cccc4cc(-c5cnn(C)c5)ncc34)nn2C2CCN(C3CCC4(CC3)CN(c3ccc(C(=O)O)cc3)C4)CC2)C1 ; CC(=O)N1CCc2c(c(N3CCCc4cc(-c5cnn(C)c5)c(C)cc43)nn2C2CCN(C3CCC4(CC3)CN(c3ccc(C(=O)O)cc3)C4)CC2)C1]

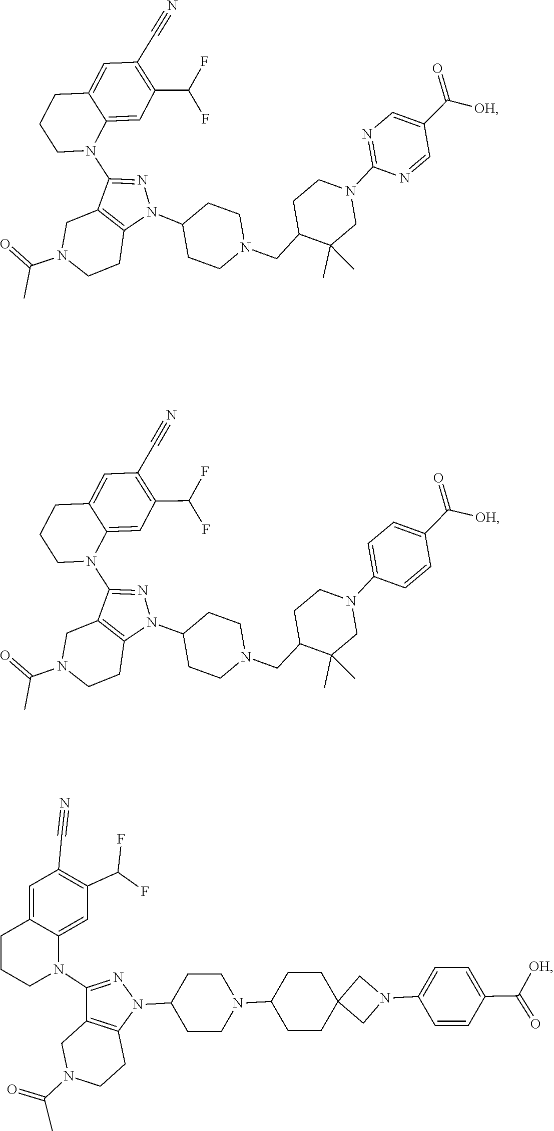 [SMILES: CC(=O)N1CCc2c(c(N3CCCc4cc(C#N)c(C(F)F)cc43)nn2C2CCN(CC3CCN(c4ncc(C(=O)O)cn4)CC3(C)C)CC2)C1 ; CC(=O)N1CCc2c(c(N3CCCc4cc(C#N)c(C(F)F)cc43)nn2C2CCN(CC3CCN(c4ccc(C(=O)O)cc4)CC3(C)C)CC2)C1 ; CC(=O)N1CCc2c(c(N3CCCc4cc(C#N)c(C(F)F)cc43)nn2C2CCN(C3CCC4(CC3)CN(c3ccc(C(=O)O)cc3)C4)CC2)C1]

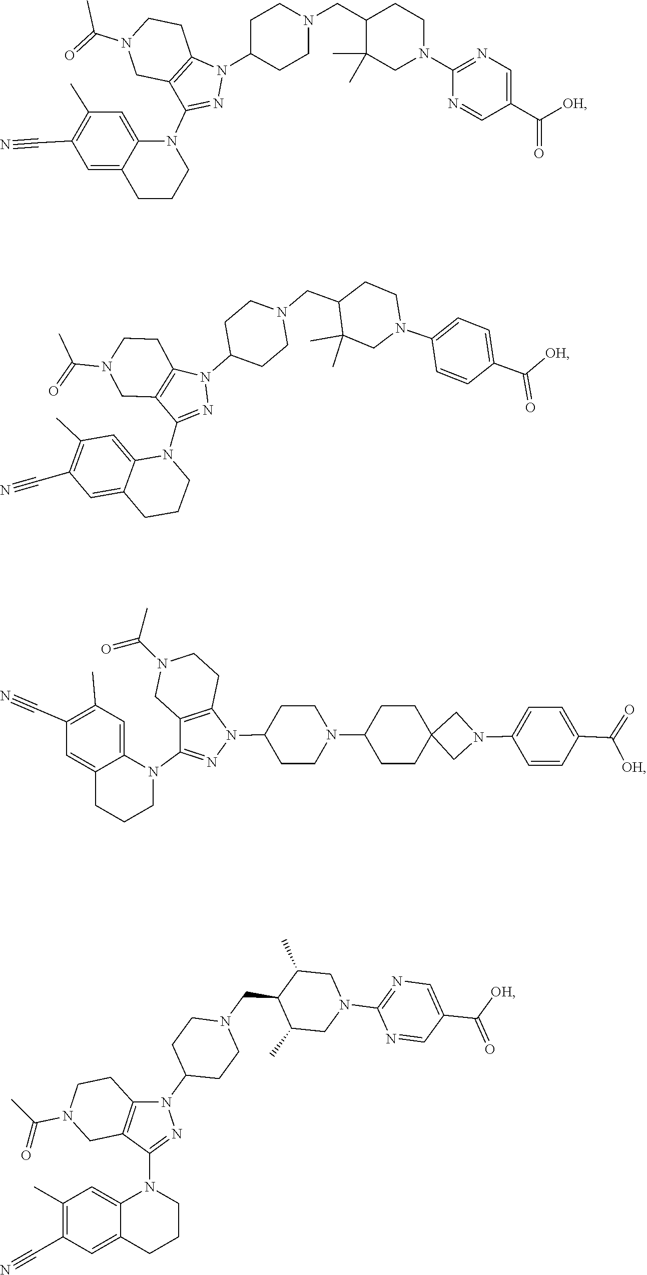 [SMILES: CC(=O)N1CCc2c(c(N3CCCc4cc(C#N)c(C)cc43)nn2C2CCN(CC3CCN(c4ncc(C(=O)O)cn4)CC3(C)C)CC2)C1 ; CC(=O)N1CCc2c(c(N3CCCc4cc(C#N)c(C)cc43)nn2C2CCN(CC3CCN(c4ccc(C(=O)O)cc4)CC3(C)C)CC2)C1 ; CC(=O)N1CCc2c(c(N3CCCc4cc(C#N)c(C)cc43)nn2C2CCN(C3CCC4(CC3)CN(c3ccc(C(=O)O)cc3)C4)CC2)C1 ; CC(=O)N1CCc2c(c(N3CCCc4cc(C#N)c(C)cc43)nn2C2CCN(C[C@H]3[C@H](C)CN(c4ncc(C(=O)O)cn4)C[C@@H]3C)CC2)C1]

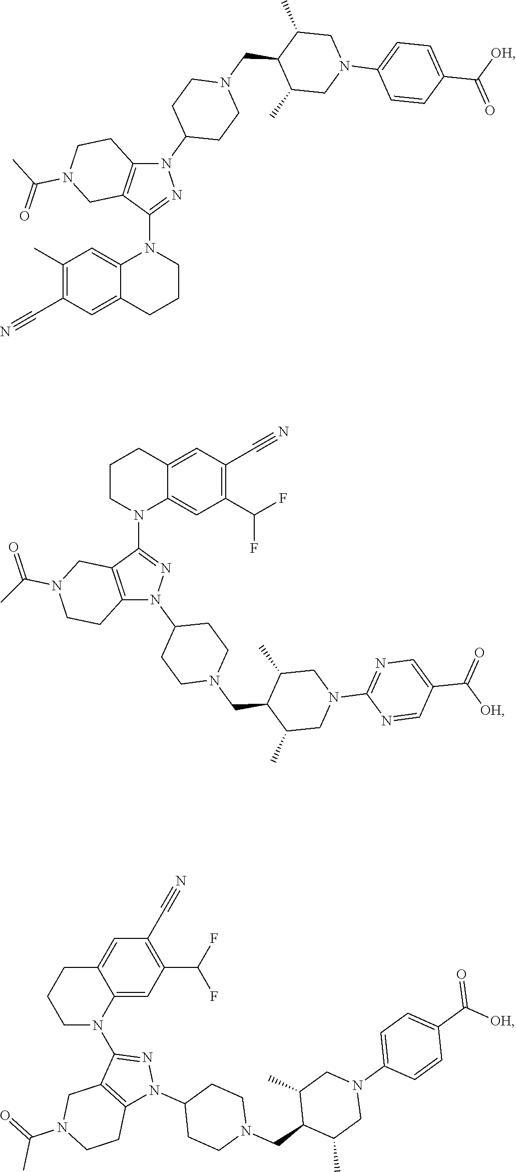 [SMILES: CC(=O)N1CCc2c(c(N3CCCc4cc(C#N)c(C)cc43)nn2C2CCN(C[C@H]3[C@H](C)CN(c4ccc(C(=O)O)cc4)C[C@@H]3C)CC2)C1 ; CC(=O)N1CCc2c(c(N3CCCc4cc(C#N)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(C#N)c(C(F)F)cc43)nn2C2CCN(C[C@H]3[C@H](C)CN(c4ccc(C(=O)O)cc4)C[C@@H]3C)CC2)C1]

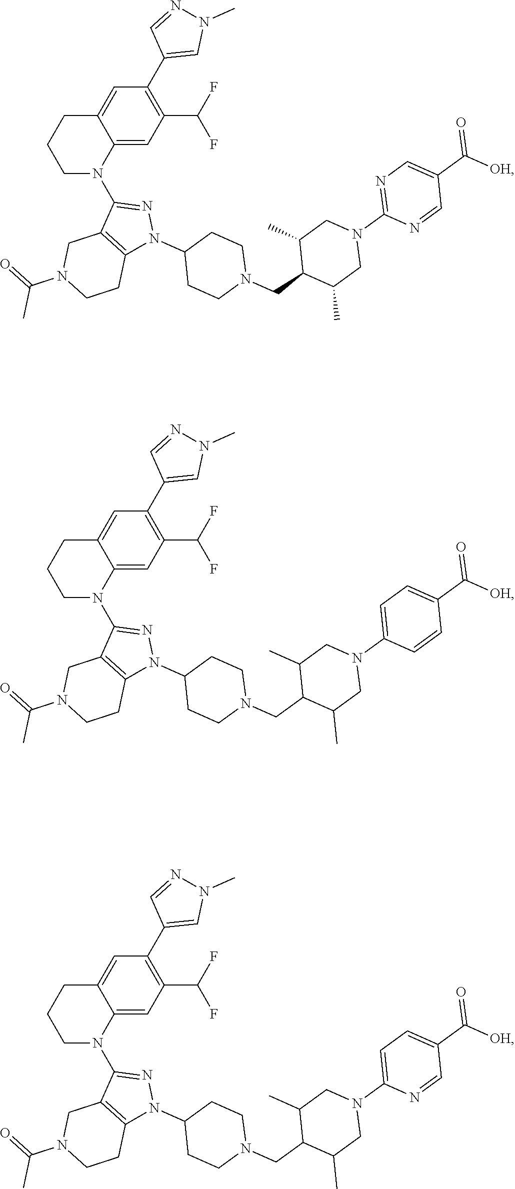 [SMILES: CC(=O)N1CCc2c(c(N3CCCc4cc(-c5cnn(C)c5)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5cnn(C)c5)c(C(F)F)cc43)nn2C2CCN(CC3C(C)CN(c4ccc(C(=O)O)cc4)CC3C)CC2)C1 ; CC(=O)N1CCc2c(c(N3CCCc4cc(-c5cnn(C)c5)c(C(F)F)cc43)nn2C2CCN(CC3C(C)CN(c4ccc(C(=O)O)cn4)CC3C)CC2)C1]

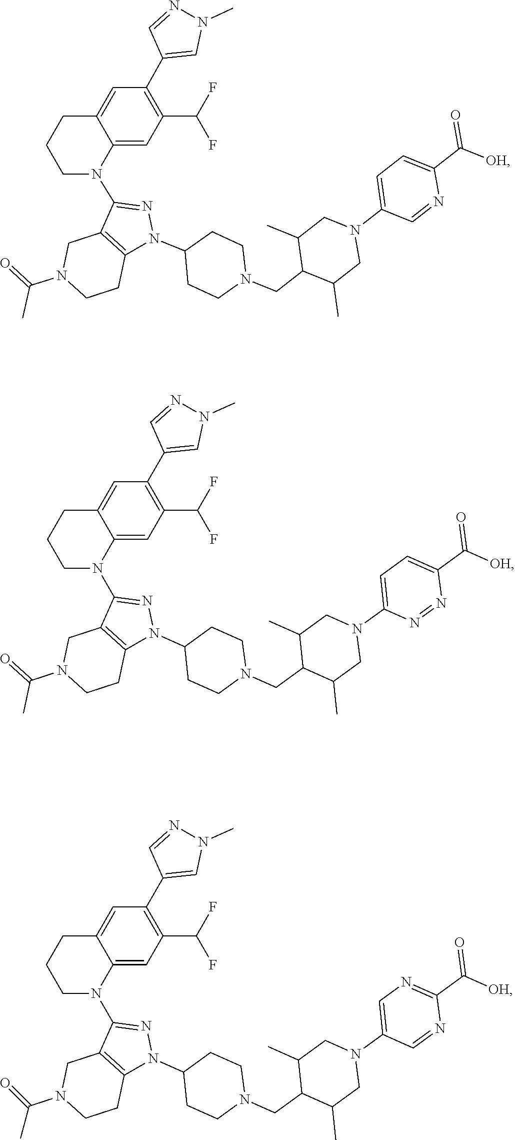 [SMILES: CC(=O)N1CCc2c(c(N3CCCc4cc(-c5cnn(C)c5)c(C(F)F)cc43)nn2C2CCN(CC3C(C)CN(c4ccc(C(=O)O)nc4)CC3C)CC2)C1 ; CC(=O)N1CCc2c(c(N3CCCc4cc(-c5cnn(C)c5)c(C(F)F)cc43)nn2C2CCN(CC3C(C)CN(c4ccc(C(=O)O)nn4)CC3C)CC2)C1 ; CC(=O)N1CCc2c(c(N3CCCc4cc(-c5cnn(C)c5)c(C(F)F)cc43)nn2C2CCN(CC3C(C)CN(c4cnc(C(=O)O)nc4)CC3C)CC2)C1]

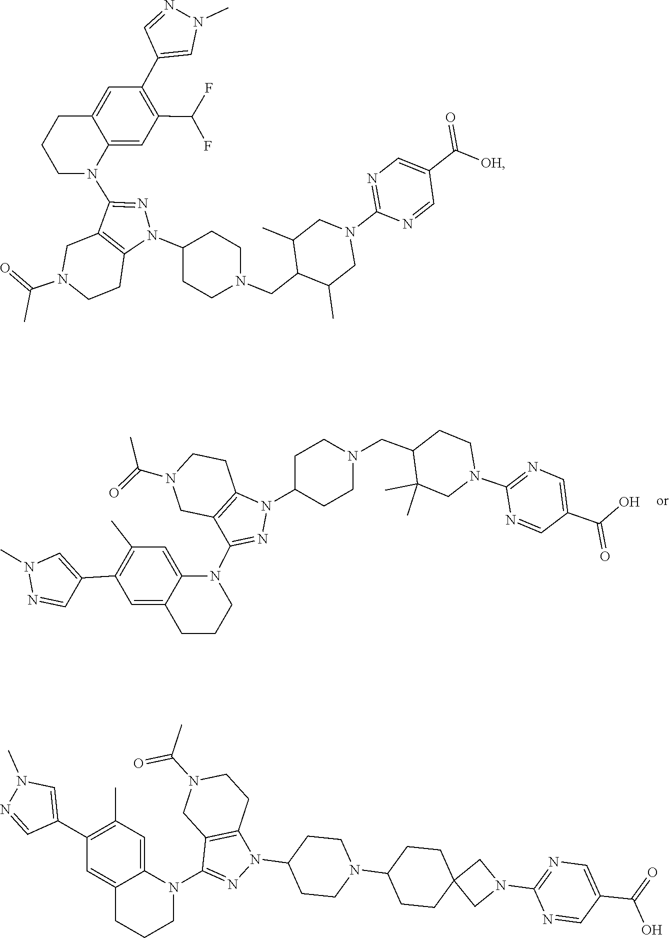 [SMILES: CC(=O)N1CCc2c(c(N3CCCc4cc(-c5cnn(C)c5)c(C(F)F)cc43)nn2C2CCN(CC3C(C)CN(c4ncc(C(=O)O)cn4)CC3C)CC2)C1 ; CC(=O)N1CCc2c(c(N3CCCc4cc(-c5cnn(C)c5)c(C)cc43)nn2C2CCN(CC3CCN(c4ncc(C(=O)O)cn4)CC3(C)C)CC2)C1 ; "or" ; CC(=O)N1CCc2c(c(N3CCCc4cc(-c5cnn(C)c5)c(C)cc43)nn2C2CCN(C3CCC4(CC3)CN(c3ncc(C(=O)O)cn3)C4)CC2)C1]

Embodiment 121a. A compound, or a pharmaceutically acceptable salt or solvate thereof, that has one of the following structures:

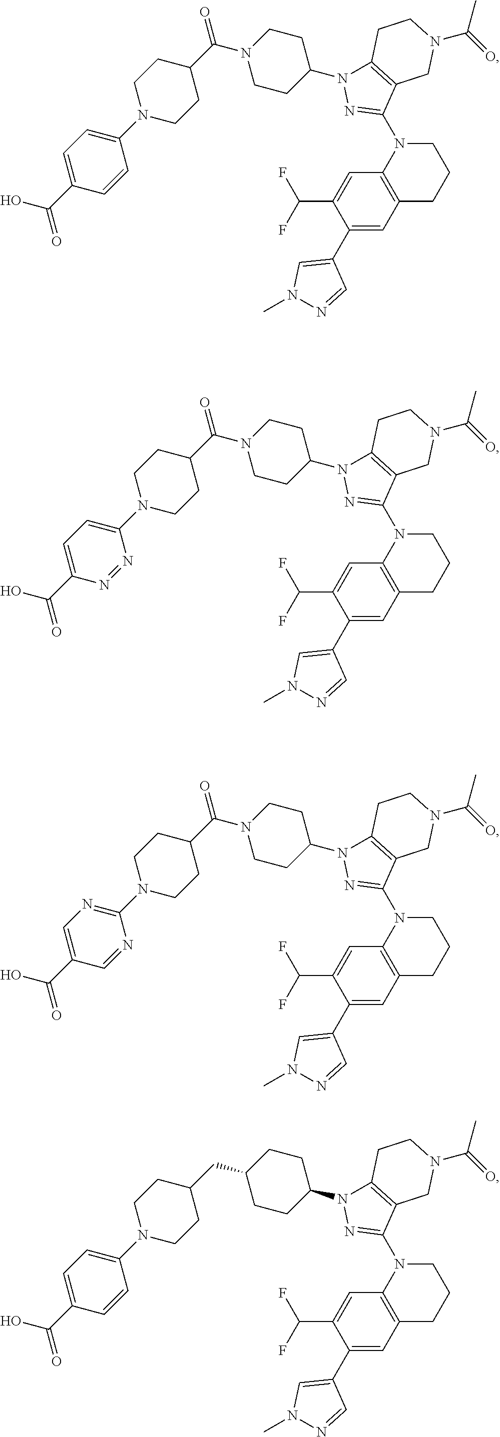 [SMILES: CC(=O)N1CCc2c(c(N3CCCc4cc(-c5cnn(C)c5)c(C(F)F)cc43)nn2C2CCN(C(=O)C3CCN(c4ccc(C(=O)O)cc4)CC3)CC2)C1 ; CC(=O)N1CCc2c(c(N3CCCc4cc(-c5cnn(C)c5)c(C(F)F)cc43)nn2C2CCN(C(=O)C3CCN(c4ccc(C(=O)O)nn4)CC3)CC2)C1 ; CC(=O)N1CCc2c(c(N3CCCc4cc(-c5cnn(C)c5)c(C(F)F)cc43)nn2C2CCN(C(=O)C3CCN(c4ncc(C(=O)O)cn4)CC3)CC2)C1 ; CC(=O)N1CCc2c(c(N3CCCc4cc(-c5cnn(C)c5)c(C(F)F)cc43)nn2[C@H]2CC[C@H](CC3CCN(c4ccc(C(=O)O)cc4)CC3)CC2)C1]

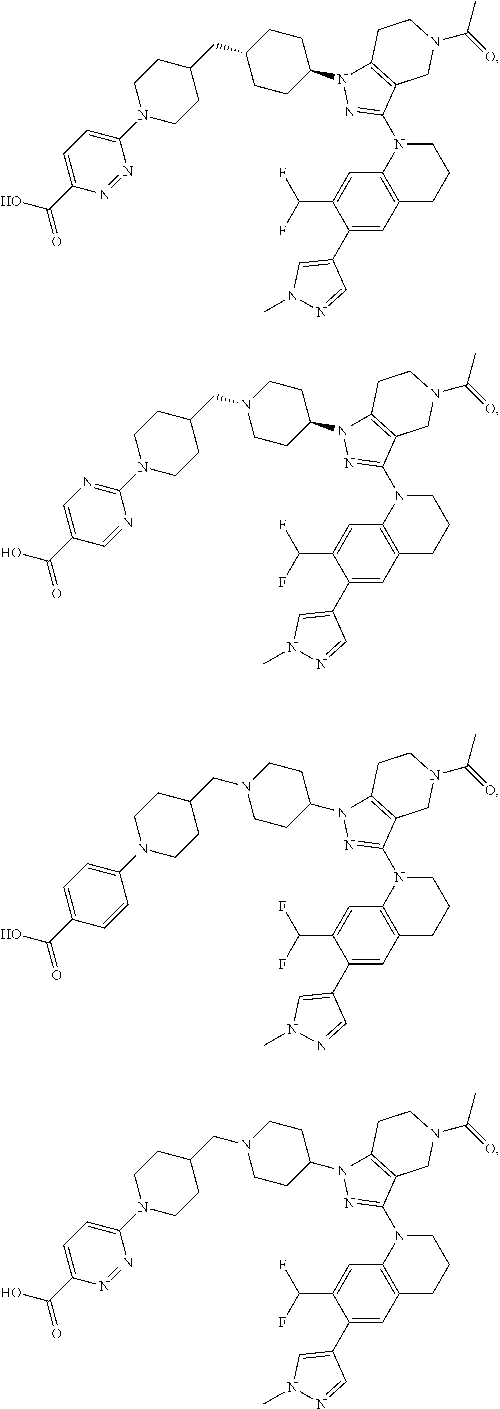

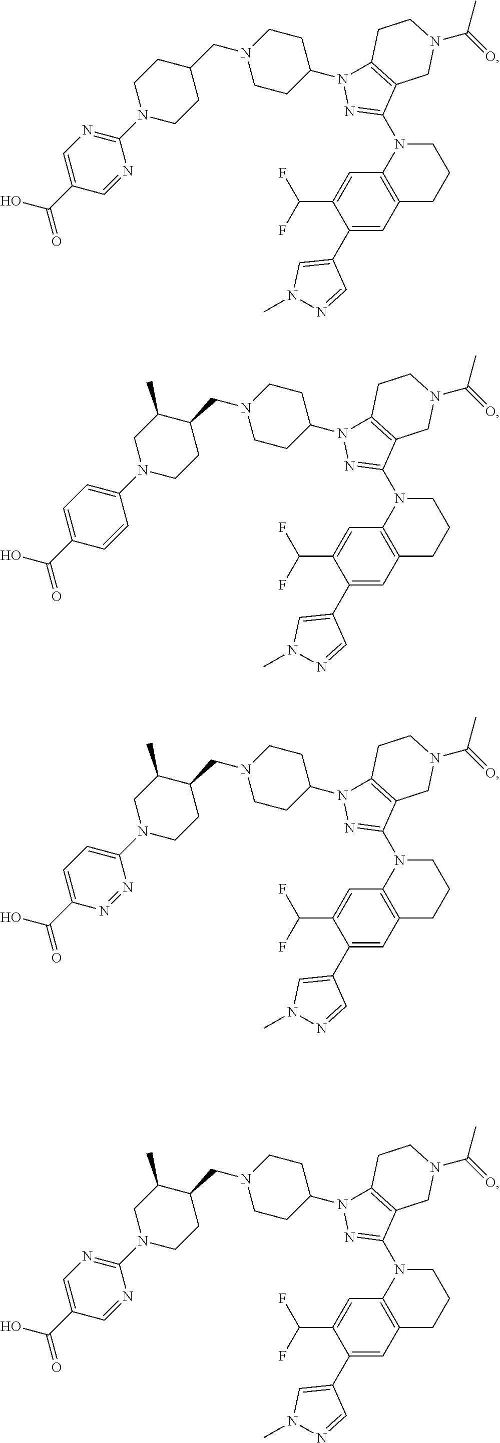 [SMILES: CC(=O)N1CCc2c(c(N3CCCc4cc(-c5cnn(C)c5)c(C(F)F)cc43)nn2C2CCN(CC3CCN(c4ncc(C(=O)O)cn4)CC3)CC2)C1 ; CC(=O)N1CCc2c(c(N3CCCc4cc(-c5cnn(C)c5)c(C(F)F)cc43)nn2C2CCN(C[C@H]3CCN(c4ccc(C(=O)O)cc4)C[C@H]3C)CC2)C1 ; CC(=O)N1CCc2c(c(N3CCCc4cc(-c5cnn(C)c5)c(C(F)F)cc43)nn2C2CCN(C[C@H]3CCN(c4ccc(C(=O)O)nn4)C[C@H]3C)CC2)C1 ; CC(=O)N1CCc2c(c(N3CCCc4cc(-c5cnn(C)c5)c(C(F)F)cc43)nn2C2CCN(C[C@H]3CCN(c4ncc(C(=O)O)cn4)C[C@H]3C)CC2)C1]

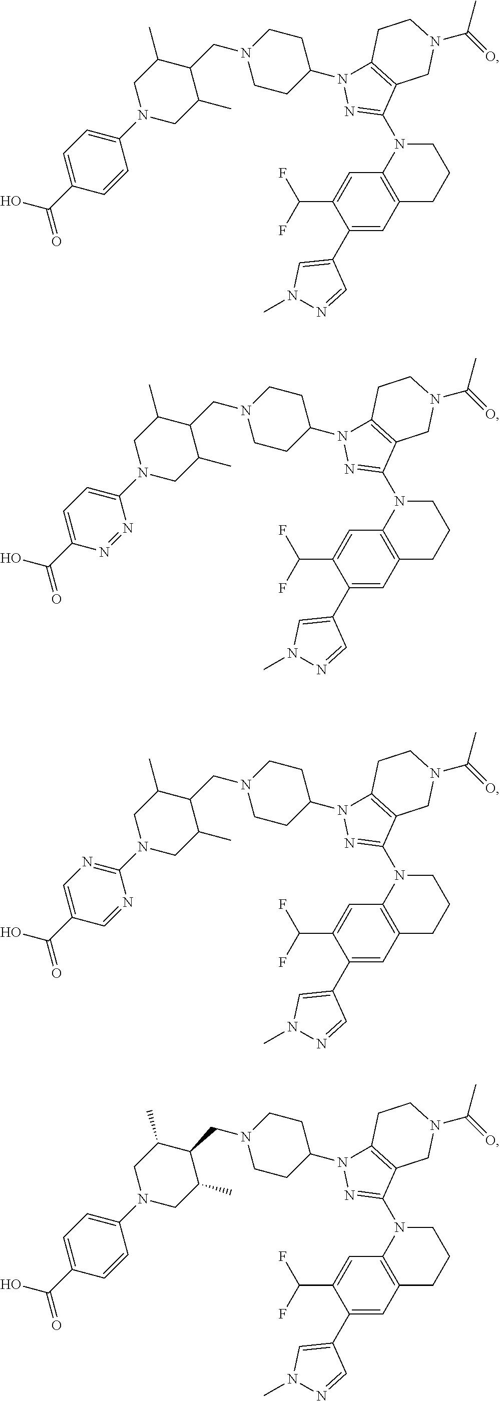 [SMILES: CC(=O)N1CCc2c(c(N3CCCc4cc(-c5cnn(C)c5)c(C(F)F)cc43)nn2C2CCN(CC3C(C)CN(c4ccc(C(=O)O)cc4)CC3C)CC2)C1 ; CC(=O)N1CCc2c(c(N3CCCc4cc(-c5cnn(C)c5)c(C(F)F)cc43)nn2C2CCN(CC3C(C)CN(c4ccc(C(=O)O)nn4)CC3C)CC2)C1 ; CC(=O)N1CCc2c(c(N3CCCc4cc(-c5cnn(C)c5)c(C(F)F)cc43)nn2C2CCN(CC3C(C)CN(c4ncc(C(=O)O)cn4)CC3C)CC2)C1 ; CC(=O)N1CCc2c(c(N3CCCc4cc(-c5cnn(C)c5)c(C(F)F)cc43)nn2C2CCN(C[C@H]3[C@H](C)CN(c4ccc(C(=O)O)cc4)C[C@@H]3C)CC2)C1]

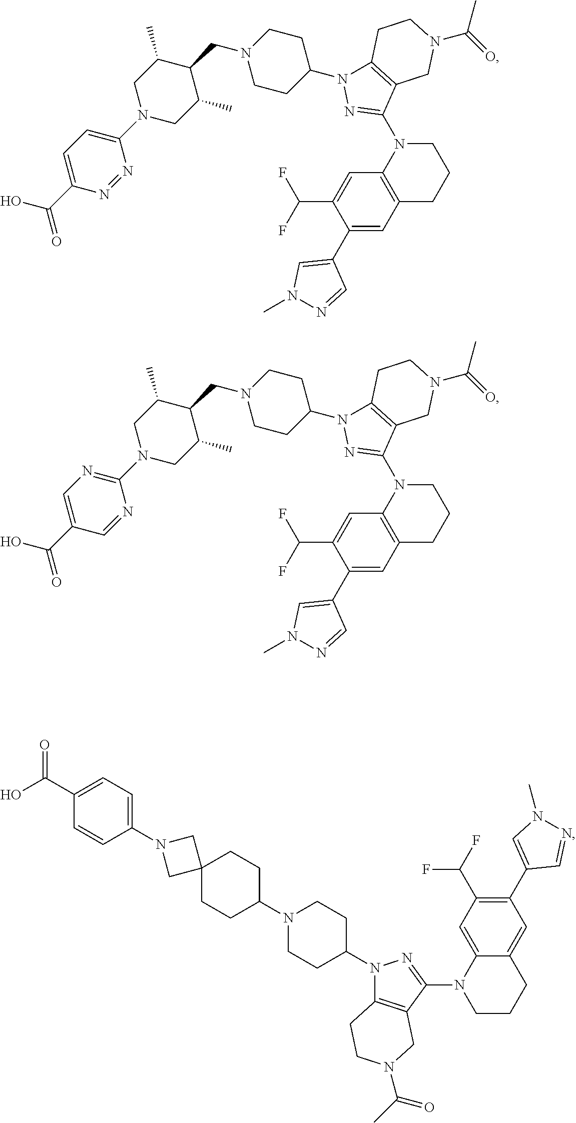 [SMILES: CC(=O)N1CCc2c(c(N3CCCc4cc(-c5cnn(C)c5)c(C(F)F)cc43)nn2C2CCN(C[C@H]3[C@H](C)CN(c4ccc(C(=O)O)nn4)C[C@@H]3C)CC2)C1 ; CC(=O)N1CCc2c(c(N3CCCc4cc(-c5cnn(C)c5)c(C(F)F)cc43)nn2C2CCN(C[C@H]3[C@H](C)CN(c4ncc(C(=O)O)cn4)C[C@@H]3C)CC2)C1 ; CC(=O)N1CCc2c(c(N3CCCc4cc(-c5cnn(C)c5)c(C(F)F)cc43)nn2C2CCN(C3CCC4(CC3)CN(c3ccc(C(=O)O)cc3)C4)CC2)C1]

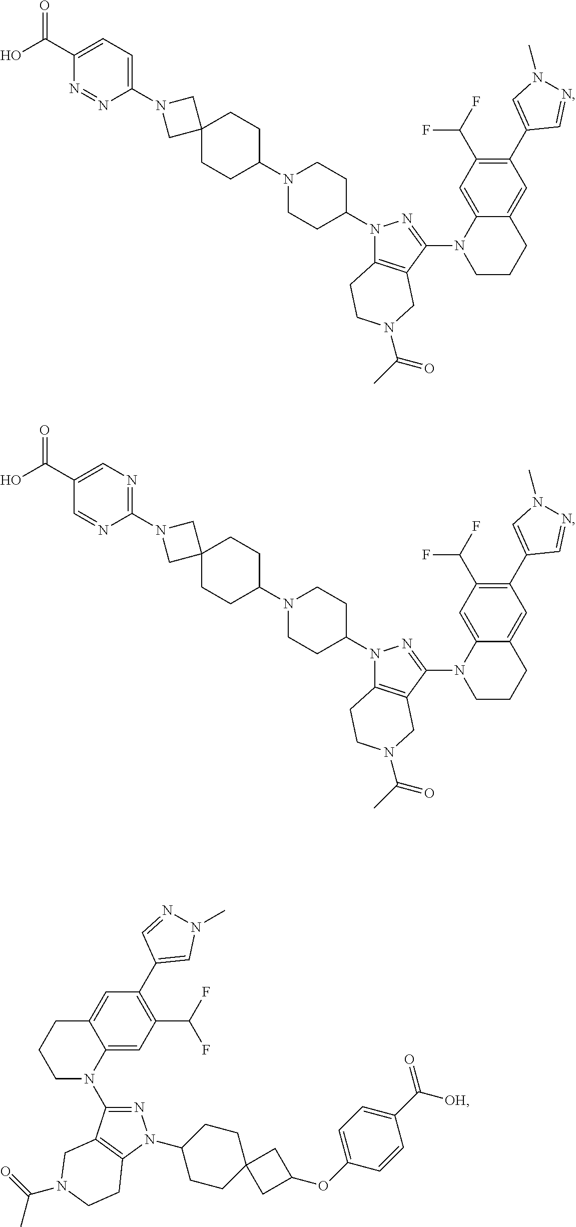 [SMILES: CC(=O)N1CCc2c(c(N3CCCc4cc(-c5cnn(C)c5)c(C(F)F)cc43)nn2C2CCN(C3CCC4(CC3)CN(c3ccc(C(=O)O)nn3)C4)CC2)C1 ; CC(=O)N1CCc2c(c(N3CCCc4cc(-c5cnn(C)c5)c(C(F)F)cc43)nn2C2CCN(C3CCC4(CC3)CN(c3ncc(C(=O)O)cn3)C4)CC2)C1 ; CC(=O)N1CCc2c(c(N3CCCc4cc(-c5cnn(C)c5)c(C(F)F)cc43)nn2C2CCC3(CC2)CC(Oc2ccc(C(=O)O)cc2)C3)C1]

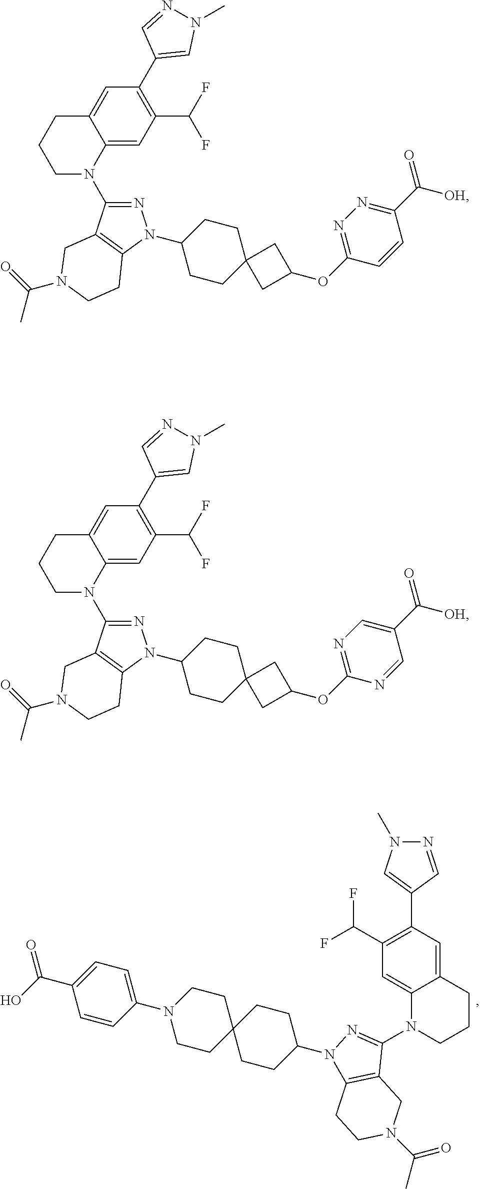 [SMILES: CC(=O)N1CCc2c(c(N3CCCc4cc(-c5cnn(C)c5)c(C(F)F)cc43)nn2C2CCC3(CC2)CC(Oc2ccc(C(=O)O)nn2)C3)C1 ; CC(=O)N1CCc2c(c(N3CCCc4cc(-c5cnn(C)c5)c(C(F)F)cc43)nn2C2CCC3(CC2)CC(Oc2ncc(C(=O)O)cn2)C3)C1 ; CC(=O)N1CCc2c(c(N3CCCc4cc(-c5cnn(C)c5)c(C(F)F)cc43)nn2C2CCC3(CC2)CCN(c2ccc(C(=O)O)cc2)CC3)C1]

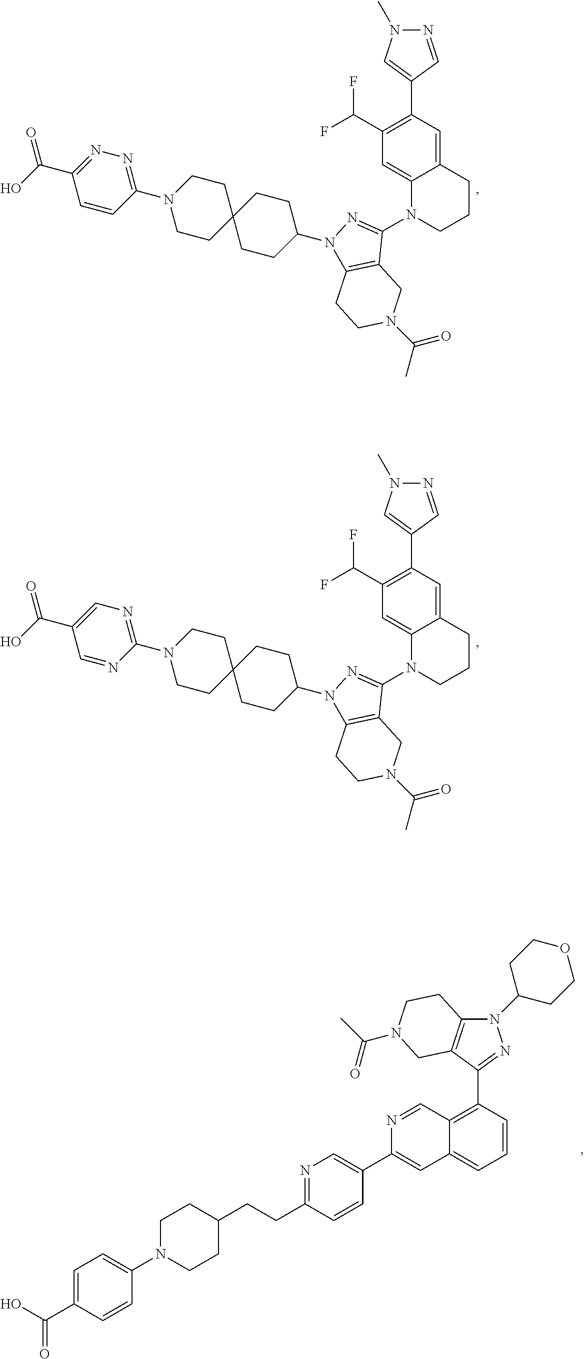 [SMILES: CC(=O)N1CCc2c(c(N3CCCc4cc(-c5cnn(C)c5)c(C(F)F)cc43)nn2C2CCC3(CC2)CCN(c2ccc(C(=O)O)nn2)CC3)C1 ; CC(=O)N1CCc2c(c(N3CCCc4cc(-c5cnn(C)c5)c(C(F)F)cc43)nn2C2CCC3(CC2)CCN(c2ncc(C(=O)O)cn2)CC3)C1 ; CC(=O)N1CCc2c(c(-c3cccc4cc(-c5ccc(CCC6CCN(c7ccc(C(=O)O)cc7)CC6)nc5)ncc34)nn2C2CCOCC2)C1]

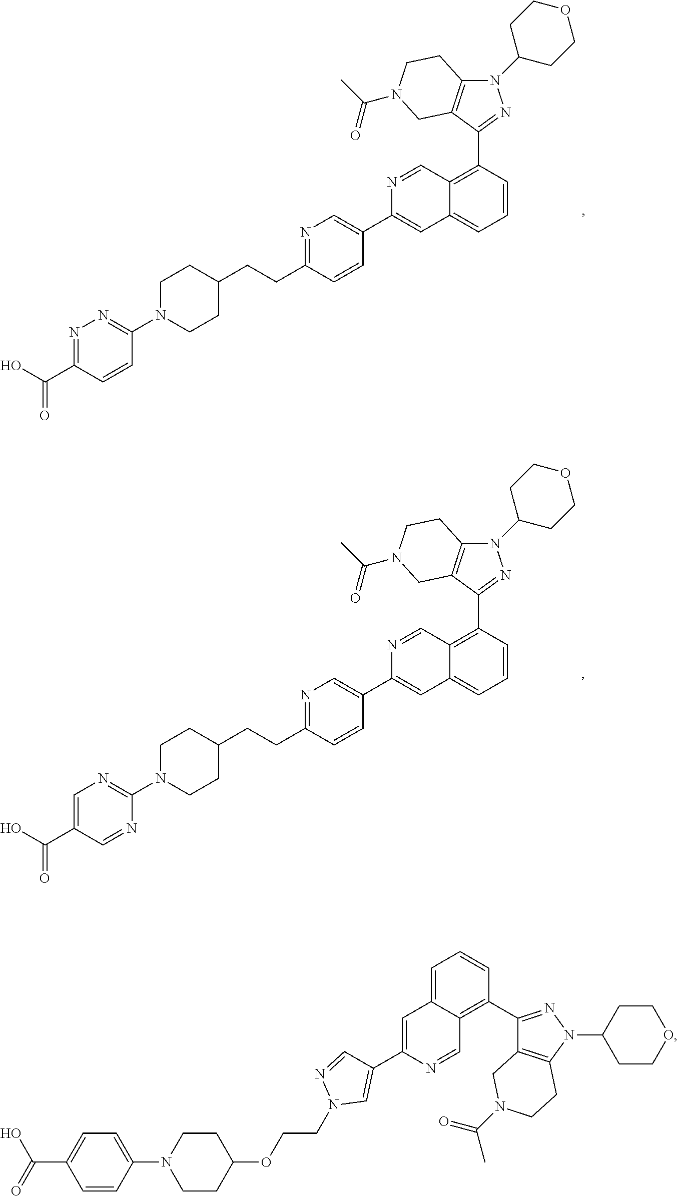 [SMILES: CC(=O)N1CCc2c(c(-c3cccc4cc(-c5ccc(CCC6CCN(c7ccc(C(=O)O)nn7)CC6)nc5)ncc34)nn2C2CCOCC2)C1 ; CC(=O)N1CCc2c(c(-c3cccc4cc(-c5ccc(CCC6CCN(c7ncc(C(=O)O)cn7)CC6)nc5)ncc34)nn2C2CCOCC2)C1 ; CC(=O)N1CCc2c(c(-c3cccc4cc(-c5cnn(CCOC6CCN(c7ccc(C(=O)O)cc7)CC6)c5)ncc34)nn2C2CCOCC2)C1]

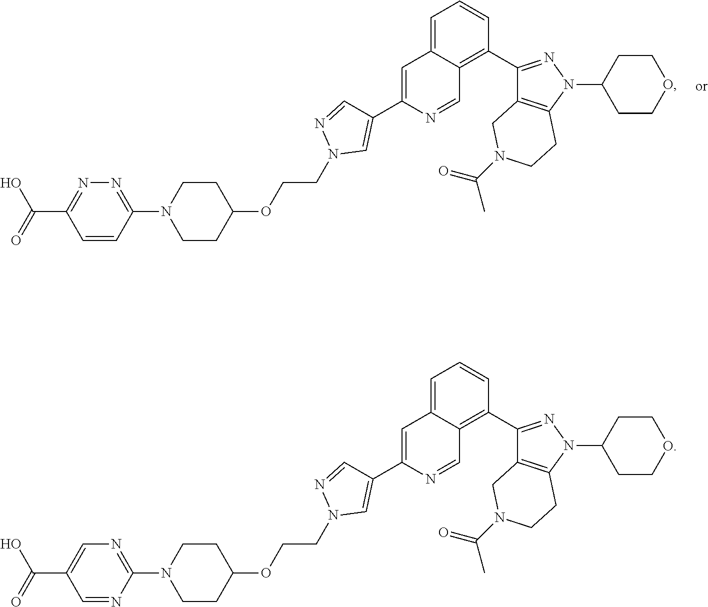 [SMILES: CC(=O)N1CCc2c(c(-c3cccc4cc(-c5cnn(CCOC6CCN(c7ccc(C(=O)O)nn7)CC6)c5)ncc34)nn2C2CCOCC2)C1 ; CC(=O)N1CCc2c(c(-c3cccc4cc(-c5cnn(CCOC6CCN(c7ncc(C(=O)O)cn7)CC6)c5)ncc34)nn2C2CCOCC2)C1]

Embodiment 122. A compound, or a pharmaceutically acceptable salt or solvate thereof, prepared by an amide coupling of the compound of embodiment 39e and the compound of embodiment 121.

Embodiment 122a. A compound, or a pharmaceutically acceptable salt or solvate thereof, prepared by an amide coupling of the compound of embodiment 39f and the compound of embodiment 121.

Embodiment 122b. A compound, or a pharmaceutically acceptable salt or solvate thereof, prepared by an amide coupling of the compound of embodiment 39g and the compound of embodiment 121.

Embodiment 123c. A compound, or a pharmaceutically acceptable salt or solvate thereof, prepared by an amide coupling of the compound of embodiment 39e and the compound of embodiment 121a.

Embodiment 123d. A compound, or a pharmaceutically acceptable salt or solvate thereof, prepared by an amide coupling of the compound of embodiment 39f and the compound of embodiment 121a.

Embodiment 123e. A compound, or a pharmaceutically acceptable salt or solvate thereof, prepared by an amide coupling of the compound of embodiment 39g and the compound of embodiment 121a.

Embodiment 124. A compound, or a pharmaceutically acceptable salt or solvate thereof, that has one of the following structures:

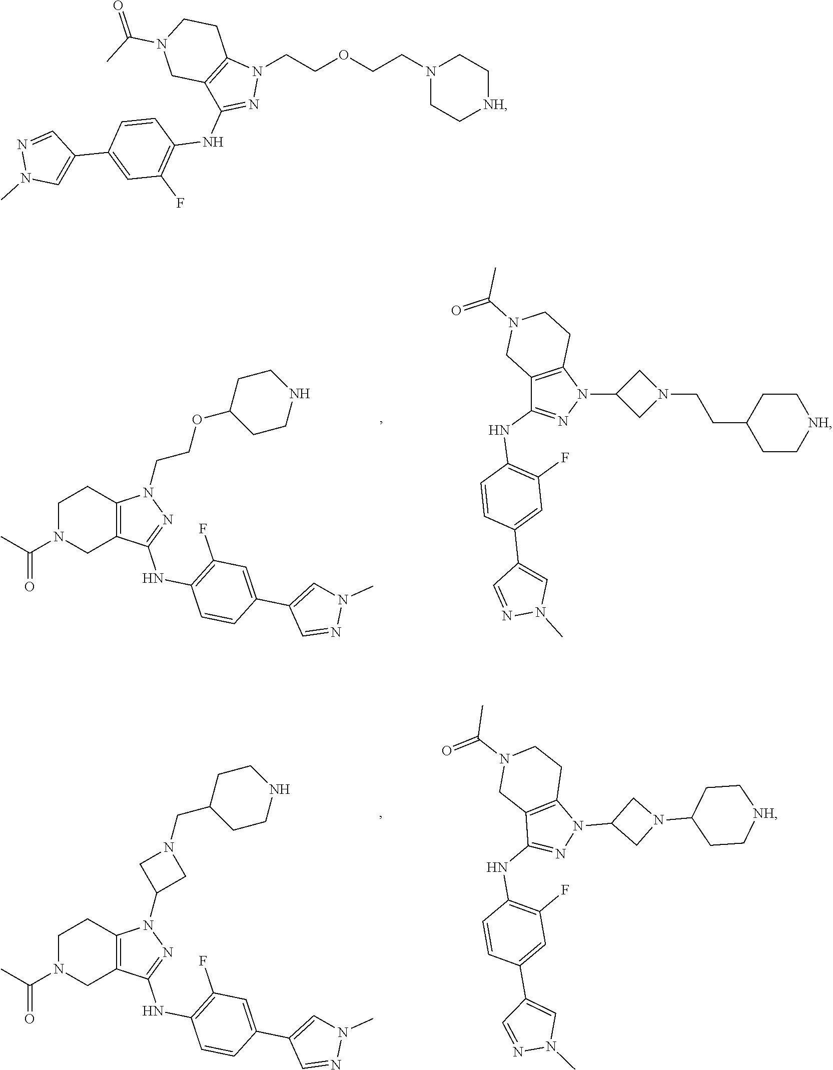 [SMILES: CC(=O)N1CCc2c(c(Nc3ccc(-c4cnn(C)c4)cc3F)nn2CCOCCN2CCNCC2)C1 ; CC(=O)N1CCc2c(c(Nc3ccc(-c4cnn(C)c4)cc3F)nn2CCOC2CCNCC2)C1 ; CC(=O)N1CCc2c(c(Nc3ccc(-c4cnn(C)c4)cc3F)nn2C2CN(CCC3CCNCC3)C2)C1 ; CC(=O)N1CCc2c(c(Nc3ccc(-c4cnn(C)c4)cc3F)nn2C2CN(CC3CCNCC3)C2)C1 ; CC(=O)N1CCc2c(c(Nc3ccc(-c4cnn(C)c4)cc3F)nn2C2CN(C3CCNCC3)C2)C1]

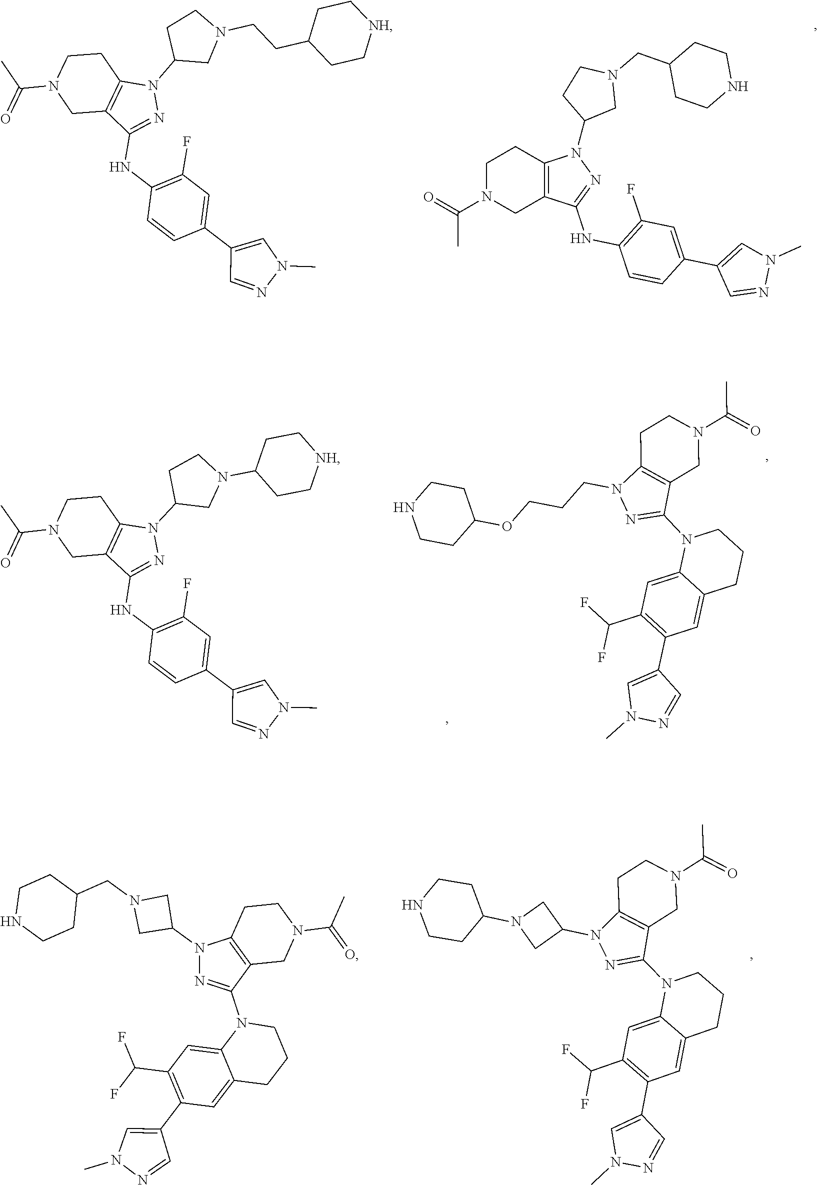 [SMILES: CC(=O)N1CCc2c(c(Nc3ccc(-c4cnn(C)c4)cc3F)nn2C2CCN(CCC3CCNCC3)C2)C1 ; CC(=O)N1CCc2c(c(Nc3ccc(-c4cnn(C)c4)cc3F)nn2C2CCN(CC3CCNCC3)C2)C1 ; CC(=O)N1CCc2c(c(Nc3ccc(-c4cnn(C)c4)cc3F)nn2C2CCN(C3CCNCC3)C2)C1 ; CC(=O)N1CCc2c(c(N3CCCc4cc(-c5cnn(C)c5)c(C(F)F)cc43)nn2CCCOC2CCNCC2)C1 ; CC(=O)N1CCc2c(c(N3CCCc4cc(-c5cnn(C)c5)c(C(F)F)cc43)nn2C2CN(CC3CCNCC3)C2)C1 ; CC(=O)N1CCc2c(c(N3CCCc4cc(-c5cnn(C)c5)c(C(F)F)cc43)nn2C2CN(C3CCNCC3)C2)C1]

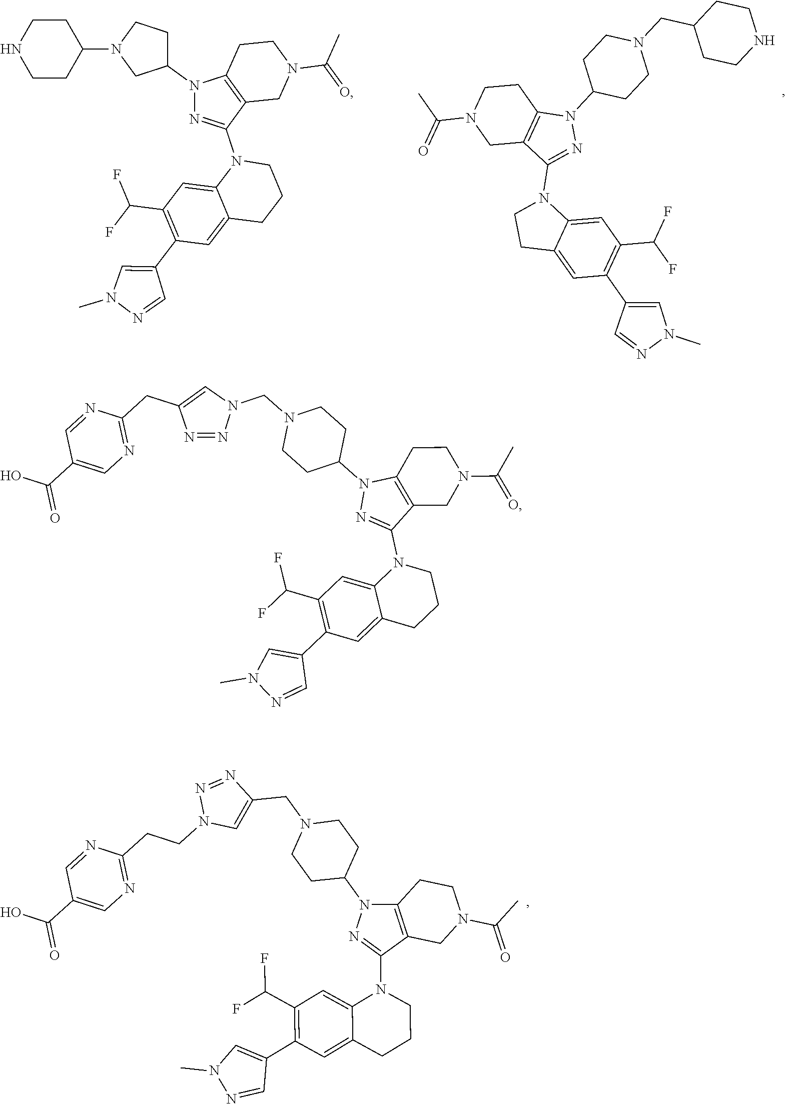 [SMILES: CC(=O)N1CCc2c(c(N3CCCc4cc(-c5cnn(C)c5)c(C(F)F)cc43)nn2C2CCN(C3CCNCC3)C2)C1 ; CC(=O)N1CCc2c(c(N3CCc4cc(-c5cnn(C)c5)c(C(F)F)cc43)nn2C2CCN(CC3CCNCC3)CC2)C1 ; CC(=O)N1CCc2c(c(N3CCCc4cc(-c5cnn(C)c5)c(C(F)F)cc43)nn2C2CCN(Cn3cc(Cc4ncc(C(=O)O)cn4)nn3)CC2)C1 ; CC(=O)N1CCc2c(c(N3CCCc4cc(-c5cnn(C)c5)c(C(F)F)cc43)nn2C2CCN(Cc3cn(CCc4ncc(C(=O)O)cn4)nn3)CC2)C1]

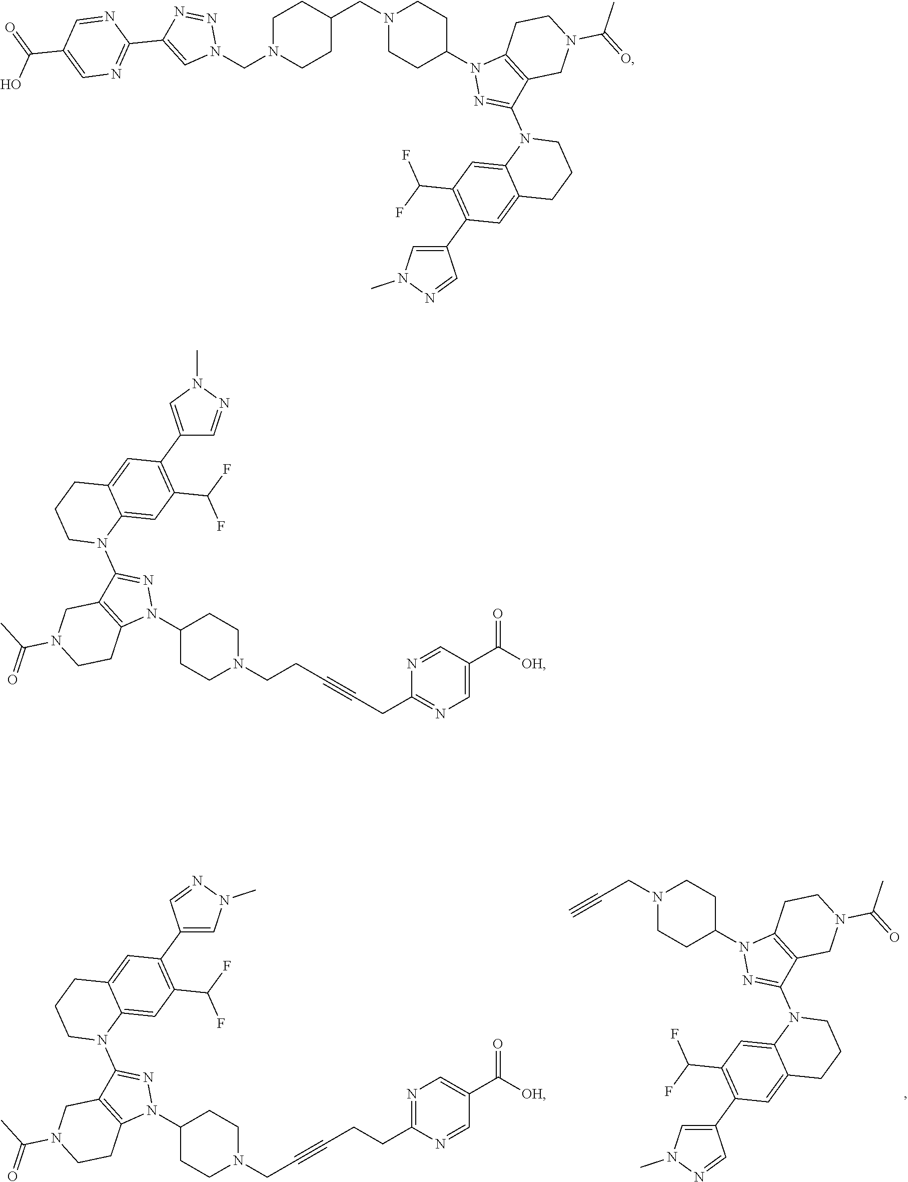 [SMILES: CC(=O)N1CCc2c(c(N3CCCc4cc(-c5cnn(C)c5)c(C(F)F)cc43)nn2C2CCN(CC3CCN(Cn4cc(-c5ncc(C(=O)O)cn5)nn4)CC3)CC2)C1 ; CC(=O)N1CCc2c(c(N3CCCc4cc(-c5cnn(C)c5)c(C(F)F)cc43)nn2C2CCN(CCC#CCc3ncc(C(=O)O)cn3)CC2)C1 ; CC(=O)N1CCc2c(c(N3CCCc4cc(-c5cnn(C)c5)c(C(F)F)cc43)nn2C2CCN(CC#CCCc3ncc(C(=O)O)cn3)CC2)C1 ; C#CCN1CCC(n2nc(N3CCCc4cc(-c5cnn(C)c5)c(C(F)F)cc43)c3c2CCN(C(C)=O)C3)CC1]

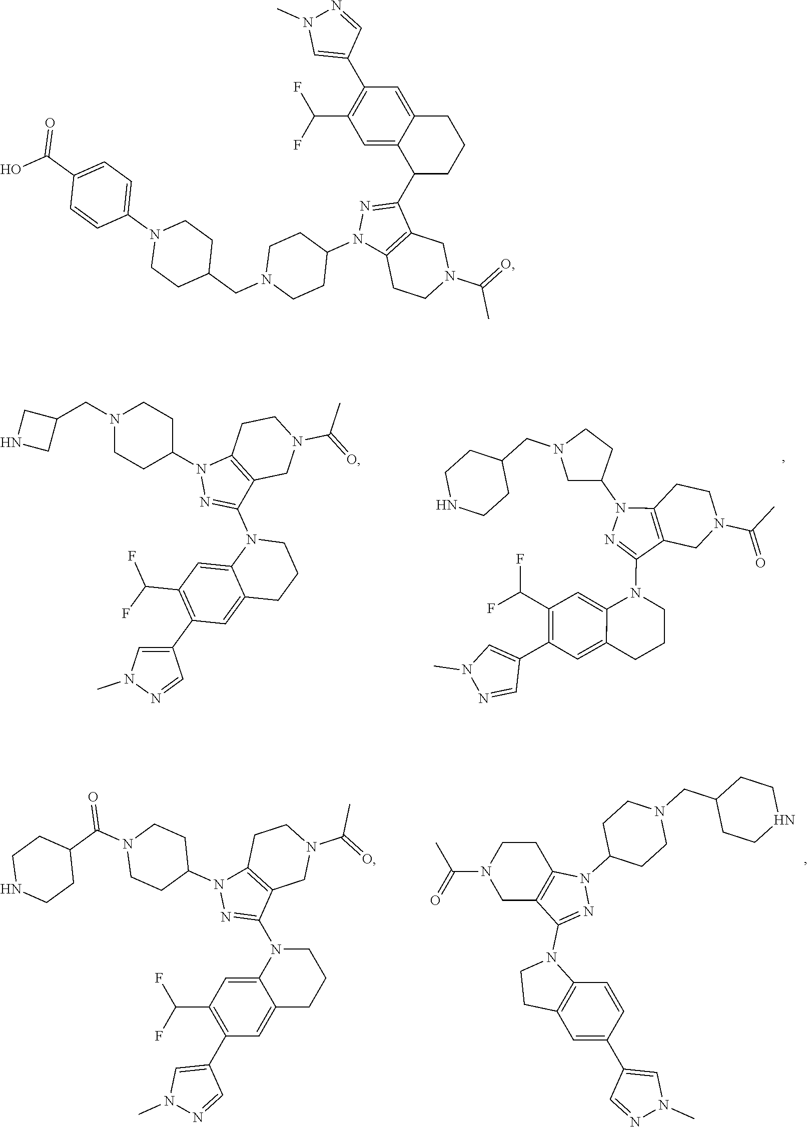 [SMILES: CC(=O)N1CCc2c(c(C3CCCc4cc(-c5cnn(C)c5)c(C(F)F)cc43)nn2C2CCN(CC3CCN(c4ccc(C(=O)O)cc4)CC3)CC2)C1 ; CC(=O)N1CCc2c(c(N3CCCc4cc(-c5cnn(C)c5)c(C(F)F)cc43)nn2C2CCN(CC3CNC3)CC2)C1 ; CC(=O)N1CCc2c(c(N3CCCc4cc(-c5cnn(C)c5)c(C(F)F)cc43)nn2C2CCN(CC3CCNCC3)C2)C1 ; CC(=O)N1CCc2c(c(N3CCCc4cc(-c5cnn(C)c5)c(C(F)F)cc43)nn2C2CCN(C(=O)C3CCNCC3)CC2)C1 ; CC(=O)N1CCc2c(c(N3CCc4cc(-c5cnn(C)c5)ccc43)nn2C2CCN(CC3CCNCC3)CC2)C1]

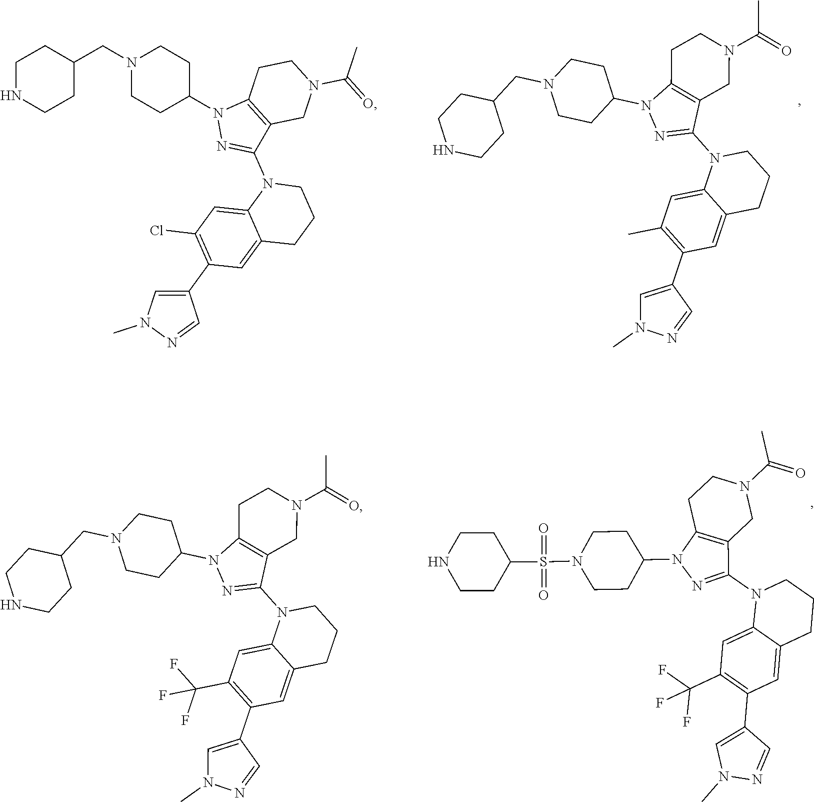 [SMILES: CC(=O)N1CCc2c(c(N3CCCc4cc(-c5cnn(C)c5)c(Cl)cc43)nn2C2CCN(CC3CCNCC3)CC2)C1 ; CC(=O)N1CCc2c(c(N3CCCc4cc(-c5cnn(C)c5)c(C)cc43)nn2C2CCN(CC3CCNCC3)CC2)C1 ; CC(=O)N1CCc2c(c(N3CCCc4cc(-c5cnn(C)c5)c(C(F)(F)F)cc43)nn2C2CCN(CC3CCNCC3)CC2)C1 ; CC(=O)N1CCc2c(c(N3CCCc4cc(-c5cnn(C)c5)c(C(F)(F)F)cc43)nn2C2CCN(S(=O)(=O)C3CCNCC3)CC2)C1]

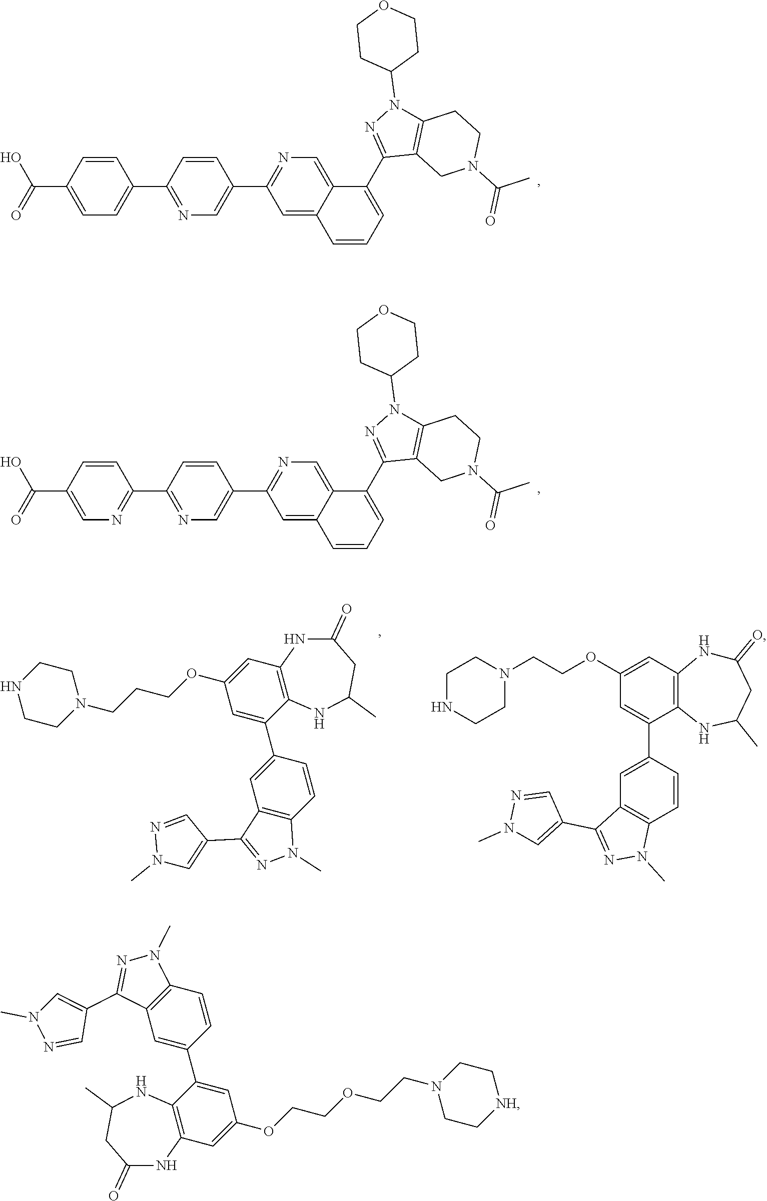

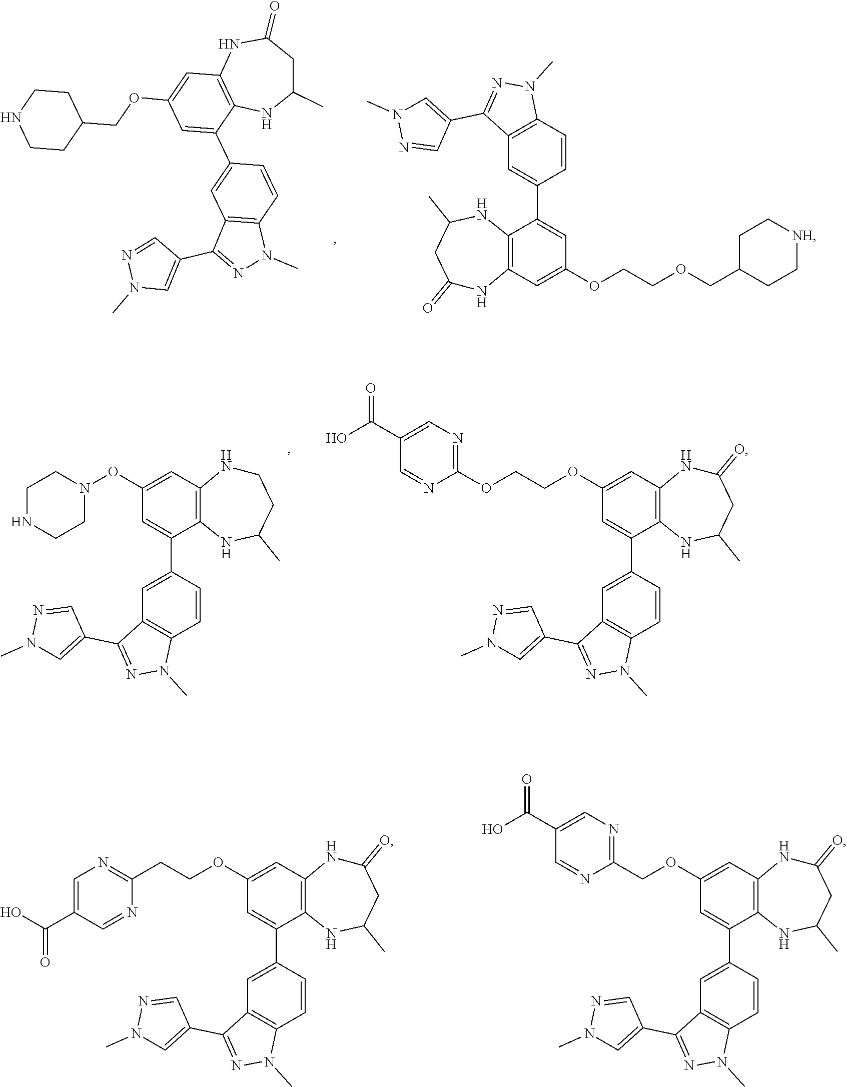

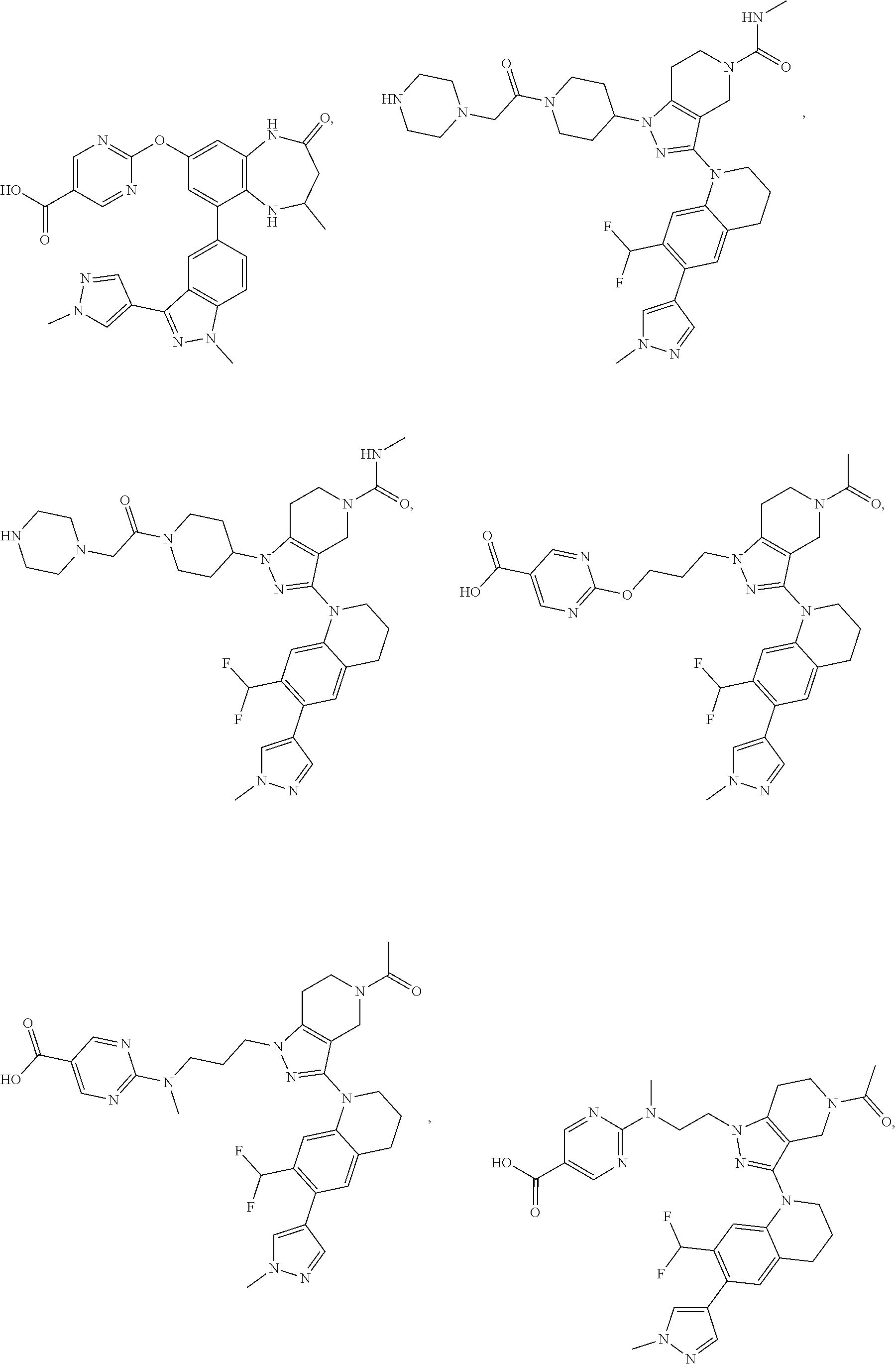 [SMILES: CC1CC(=O)Nc2cc(Oc3ncc(C(=O)O)cn3)cc(-c3ccc4c(c3)c(-c3cnn(C)c3)nn4C)c2N1 ; CNC(=O)N1CCc2c(c(N3CCCc4cc(-c5cnn(C)c5)c(C(F)F)cc43)nn2C2CCN(C(=O)CN3CCNCC3)CC2)C1 ; CNC(=O)N1CCc2c(c(N3CCCc4cc(-c5cnn(C)c5)c(C(F)F)cc43)nn2C2CCN(C(=O)CN3CCNCC3)CC2)C1 ; CC(=O)N1CCc2c(c(N3CCCc4cc(-c5cnn(C)c5)c(C(F)F)cc43)nn2CCCOc2ncc(C(=O)O)cn2)C1 ; CC(=O)N1CCc2c(c(N3CCCc4cc(-c5cnn(C)c5)c(C(F)F)cc43)nn2CCCN(C)c2ncc(C(=O)O)cn2)C1 ; CC(=O)N1CCc2c(c(N3CCCc4cc(-c5cnn(C)c5)c(C(F)F)cc43)nn2CCN(C)c2ncc(C(=O)O)cn2)C1]

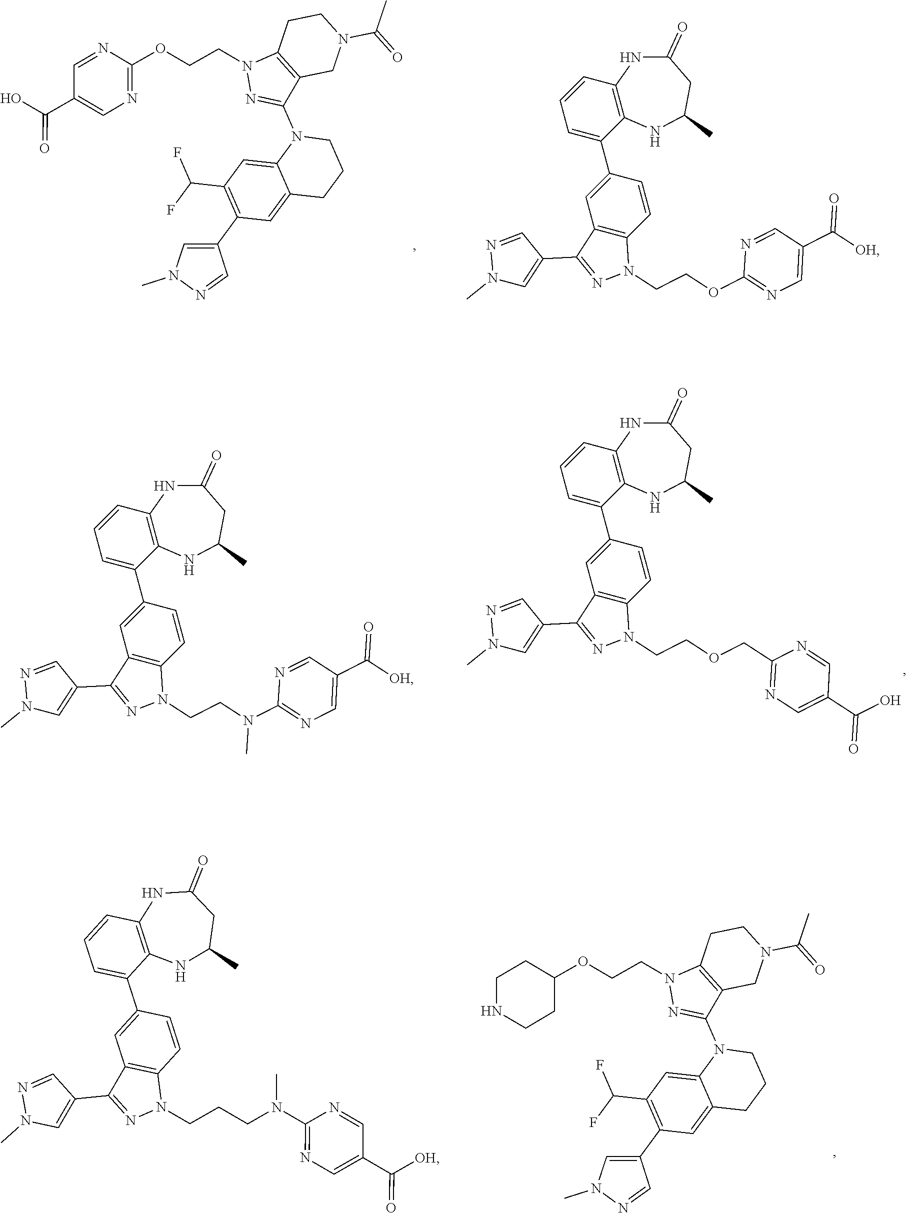 [SMILES: CC(=O)N1CCc2c(c(N3CCCc4cc(-c5cnn(C)c5)c(C(F)F)cc43)nn2CCOc2ncc(C(=O)O)cn2)C1 ; C[C@@H]1CC(=O)Nc2cccc(-c3ccc4c(c3)c(-c3cnn(C)c3)nn4CCOc3ncc(C(=O)O)cn3)c2N1 ; C[C@@H]1CC(=O)Nc2cccc(-c3ccc4c(c3)c(-c3cnn(C)c3)nn4CCN(C)c3ncc(C(=O)O)cn3)c2N1 ; C[C@@H]1CC(=O)Nc2cccc(-c3ccc4c(c3)c(-c3cnn(C)c3)nn4CCOCc3ncc(C(=O)O)cn3)c2N1 ; C[C@@H]1CC(=O)Nc2cccc(-c3ccc4c(c3)c(-c3cnn(C)c3)nn4CCCN(C)c3ncc(C(=O)O)cn3)c2N1 ; CC(=O)N1CCc2c(c(N3CCCc4cc(-c5cnn(C)c5)c(C(F)F)cc43)nn2CCOC2CCNCC2)C1]

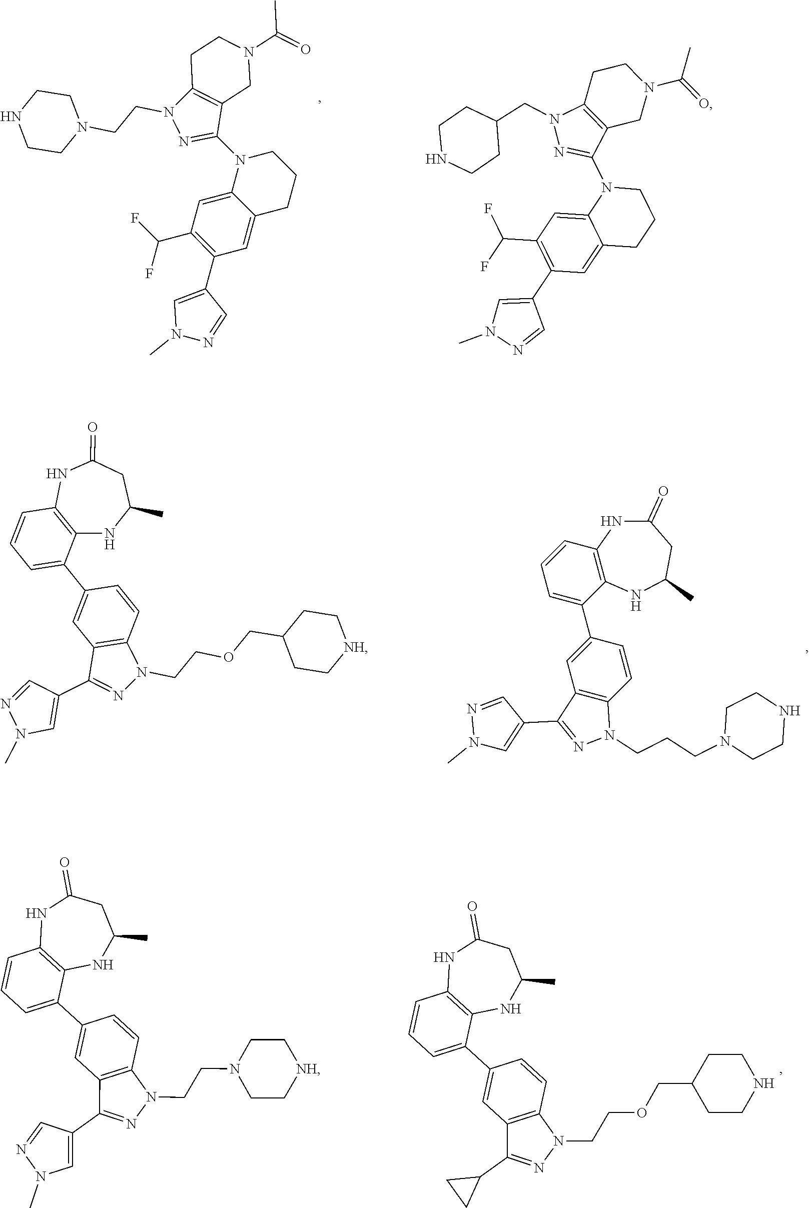 [SMILES: CC(=O)N1CCc2c(c(N3CCCc4cc(-c5cnn(C)c5)c(C(F)F)cc43)nn2CCN2CCNCC2)C1 ; CC(=O)N1CCc2c(c(N3CCCc4cc(-c5cnn(C)c5)c(C(F)F)cc43)nn2CC2CCNCC2)C1 ; C[C@@H]1CC(=O)Nc2cccc(-c3ccc4c(c3)c(-c3cnn(C)c3)nn4CCOCC3CCNCC3)c2N1 ; C[C@@H]1CC(=O)Nc2cccc(-c3ccc4c(c3)c(-c3cnn(C)c3)nn4CCCN3CCNCC3)c2N1 ; C[C@@H]1CC(=O)Nc2cccc(-c3ccc4c(c3)c(-c3cnn(C)c3)nn4CCN3CCNCC3)c2N1 ; C[C@@H]1CC(=O)Nc2cccc(-c3ccc4c(c3)c(C3CC3)nn4CCOCC3CCNCC3)c2N1]

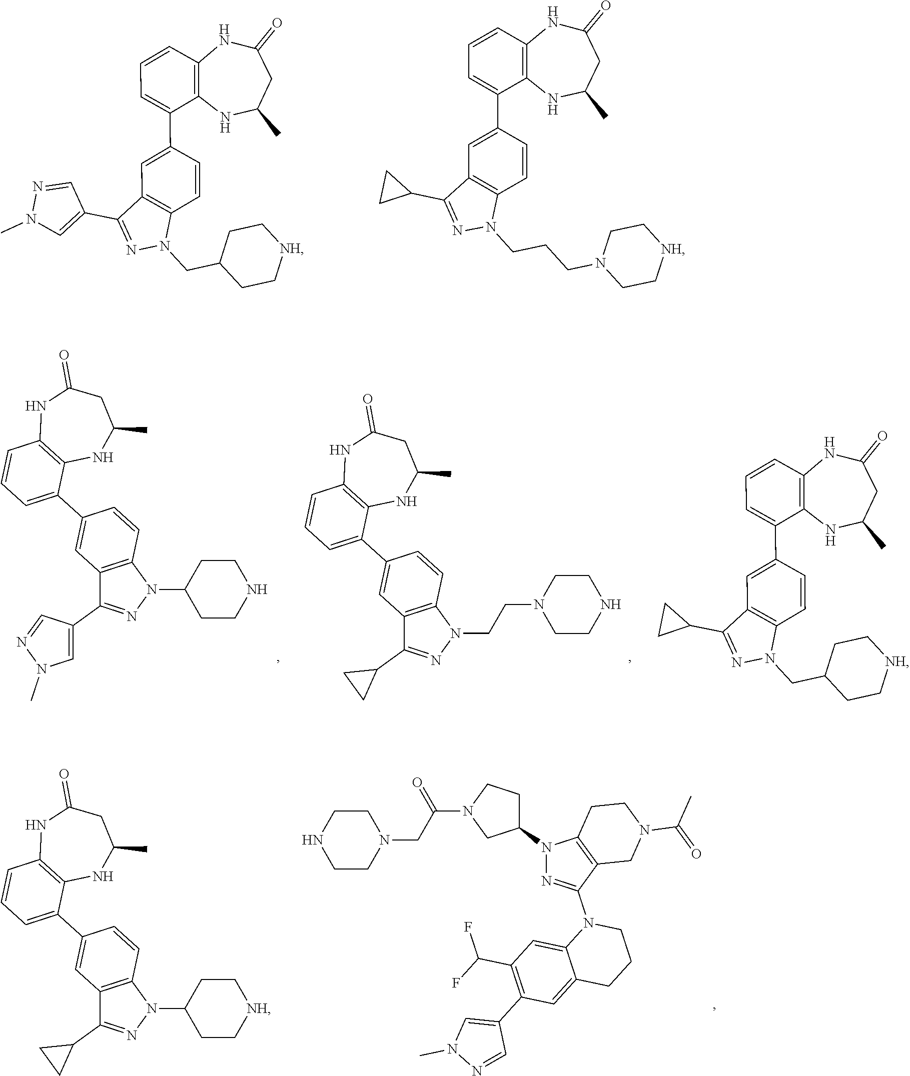

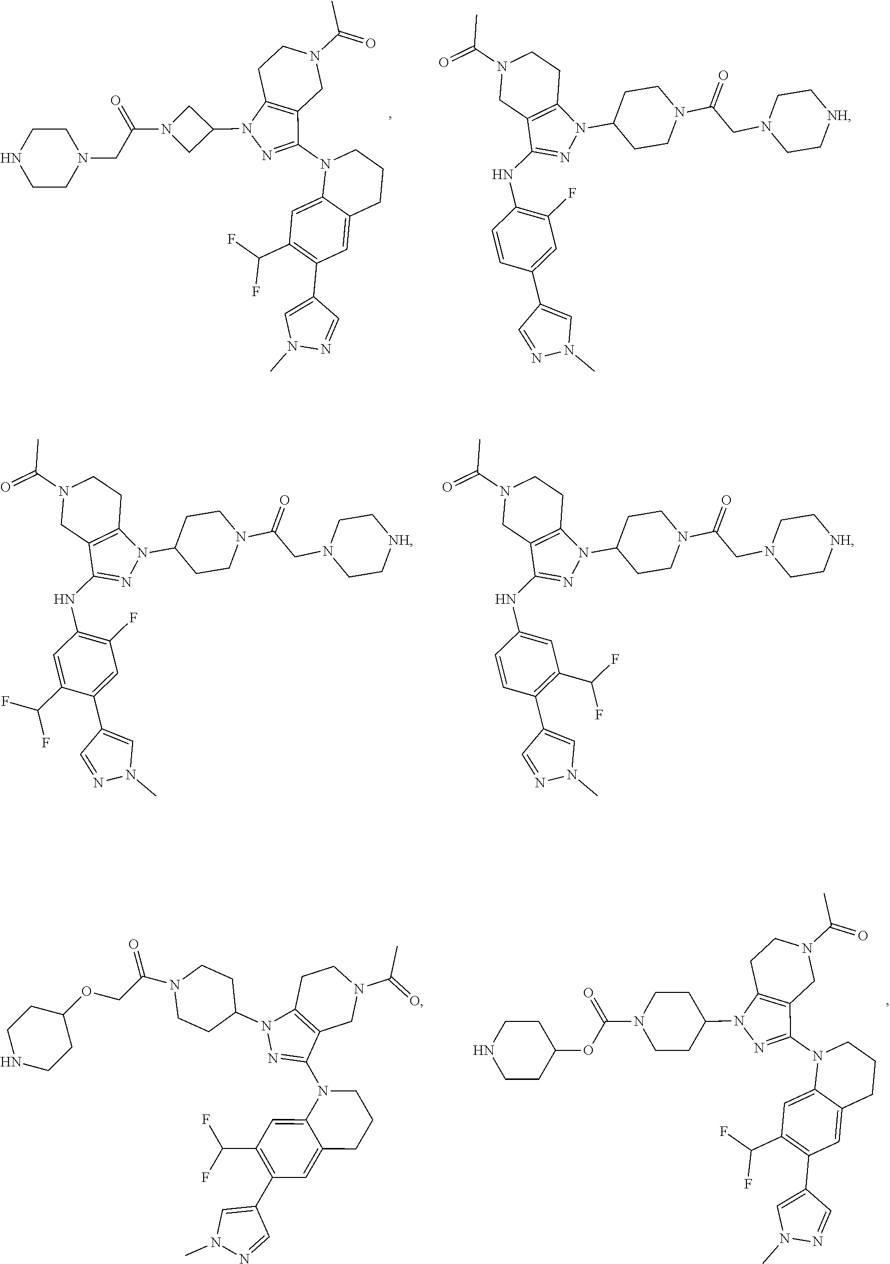 [SMILES: CC(=O)N1CCc2c(c(N3CCCc4cc(-c5cnn(C)c5)c(C(F)F)cc43)nn2C2CN(C(=O)CN3CCNCC3)C2)C1 ; CC(=O)N1CCc2c(c(Nc3ccc(-c4cnn(C)c4)cc3F)nn2C2CCN(C(=O)CN3CCNCC3)CC2)C1 ; CC(=O)N1CCc2c(c(Nc3cc(C(F)F)c(-c4cnn(C)c4)cc3F)nn2C2CCN(C(=O)CN3CCNCC3)CC2)C1 ; CC(=O)N1CCc2c(c(Nc3ccc(-c4cnn(C)c4)c(C(F)F)c3)nn2C2CCN(C(=O)CN3CCNCC3)CC2)C1 ; CC(=O)N1CCc2c(c(N3CCCc4cc(-c5cnn(C)c5)c(C(F)F)cc43)nn2C2CCN(C(=O)COC3CCNCC3)CC2)C1 ; CC(=O)N1CCc2c(c(N3CCCc4cc(-c5cnn(C)c5)c(C(F)F)cc43)nn2C2CCN(C(=O)OC3CCNCC3)CC2)C1]

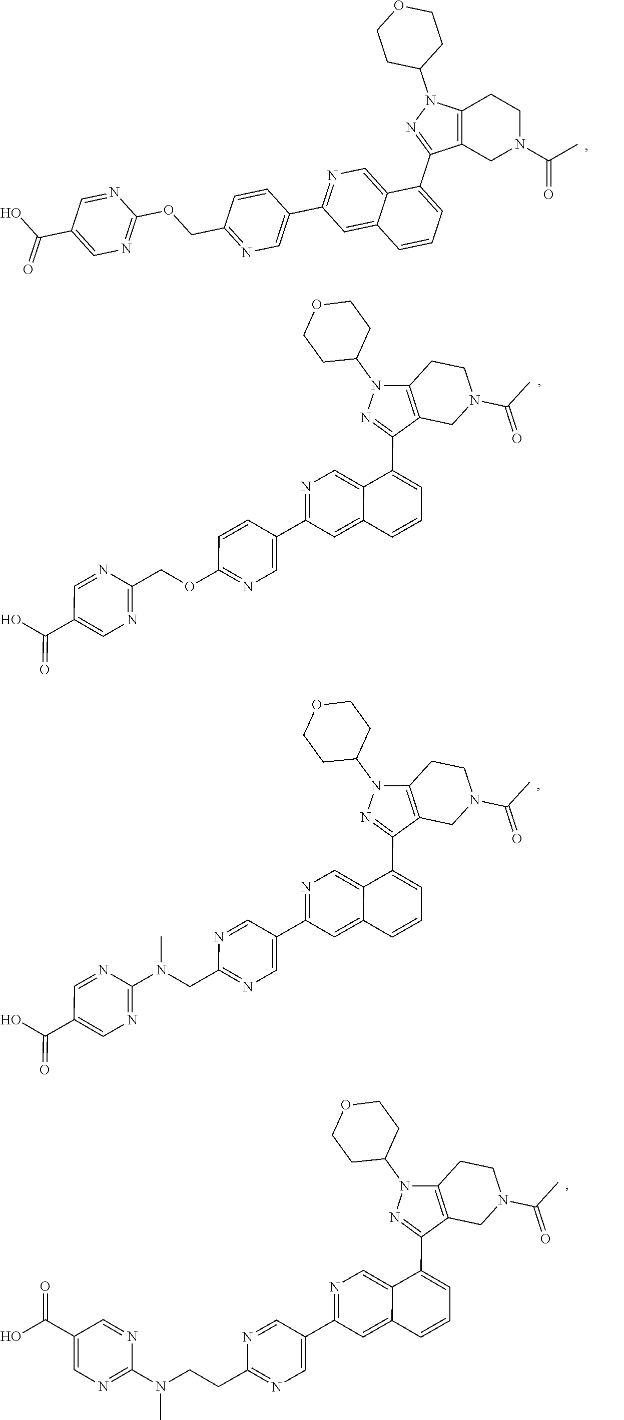 [SMILES: CC(=O)N1CCc2c(c(-c3cccc4cc(-c5ccc(COc6ncc(C(=O)O)cn6)nc5)ncc34)nn2C2CCOCC2)C1 ; CC(=O)N1CCc2c(c(-c3cccc4cc(-c5ccc(OCc6ncc(C(=O)O)cn6)nc5)ncc34)nn2C2CCOCC2)C1 ; CC(=O)N1CCc2c(c(-c3cccc4cc(-c5cnc(CN(C)c6ncc(C(=O)O)cn6)nc5)ncc34)nn2C2CCOCC2)C1 ; CC(=O)N1CCc2c(c(-c3cccc4cc(-c5cnc(CCN(C)c6ncc(C(=O)O)cn6)nc5)ncc34)nn2C2CCOCC2)C1]

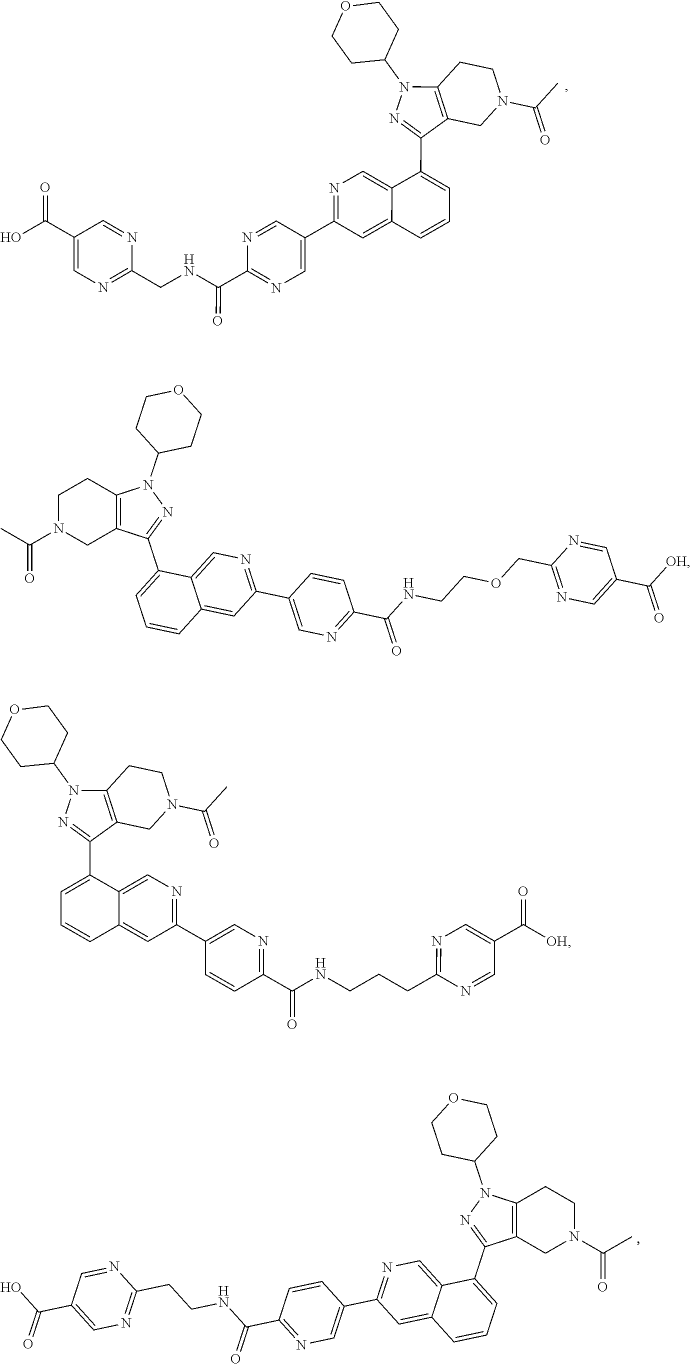

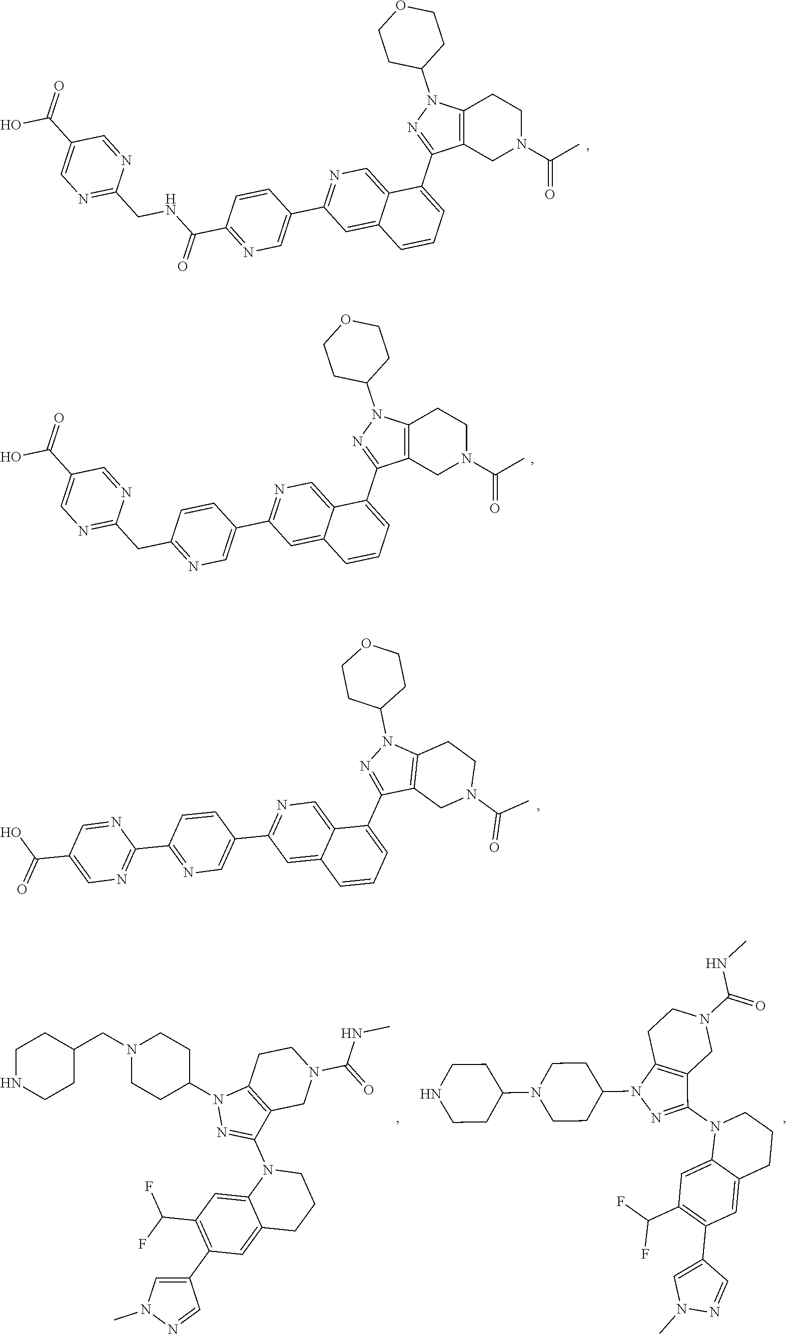 [SMILES: CC(=O)N1CCc2c(c(-c3cccc4cc(-c5ccc(C(=O)NCc6ncc(C(=O)O)cn6)nc5)ncc34)nn2C2CCOCC2)C1 ; CC(=O)N1CCc2c(c(-c3cccc4cc(-c5ccc(Cc6ncc(C(=O)O)cn6)nc5)ncc34)nn2C2CCOCC2)C1 ; CC(=O)N1CCc2c(c(-c3cccc4cc(-c5ccc(-c6ncc(C(=O)O)cn6)nc5)ncc34)nn2C2CCOCC2)C1 ; CNC(=O)N1CCc2c(c(N3CCCc4cc(-c5cnn(C)c5)c(C(F)F)cc43)nn2C2CCN(CC3CCNCC3)CC2)C1 ; CNC(=O)N1CCc2c(c(N3CCCc4cc(-c5cnn(C)c5)c(C(F)F)cc43)nn2C2CCN(C3CCNCC3)CC2)C1]

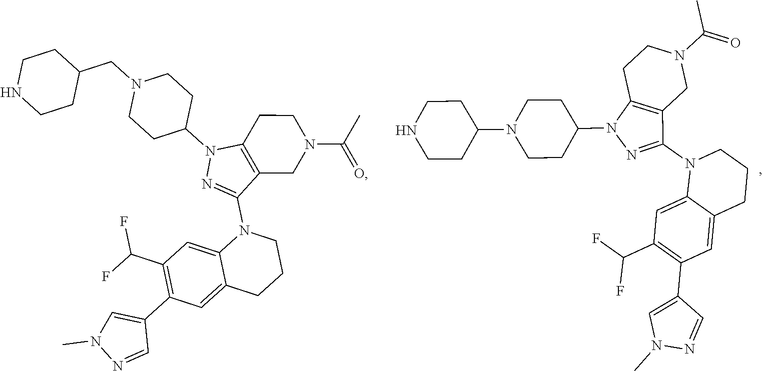 [SMILES: CC(=O)N1CCc2c(c(N3CCCc4cc(-c5cnn(C)c5)c(C(F)F)cc43)nn2C2CCN(CC3CCNCC3)CC2)C1 ; CC(=O)N1CCc2c(c(N3CCCc4cc(-c5cnn(C)c5)c(C(F)F)cc43)nn2C2CCN(C3CCNCC3)CC2)C1 ; ","]

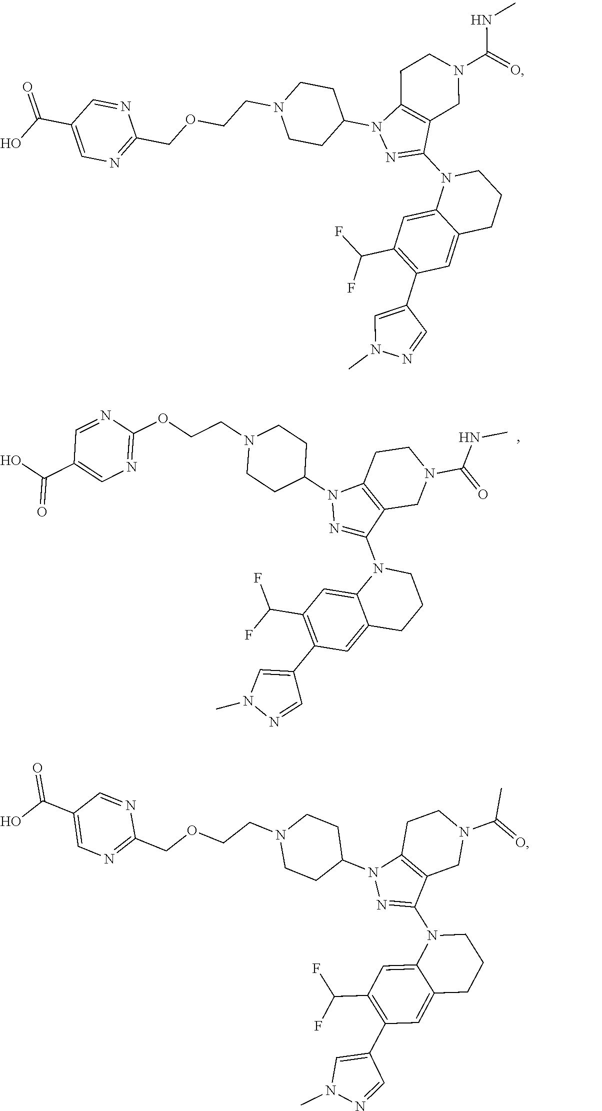 [SMILES: CNC(=O)N1CCc2c(c(N3CCCc4cc(-c5cnn(C)c5)c(C(F)F)cc43)nn2C2CCN(CCOCc3ncc(C(=O)O)cn3)CC2)C1 ; CNC(=O)N1CCc2c(c(N3CCCc4cc(-c5cnn(C)c5)c(C(F)F)cc43)nn2C2CCN(CCOc3ncc(C(=O)O)cn3)CC2)C1 ; CC(=O)N1CCc2c(c(N3CCCc4cc(-c5cnn(C)c5)c(C(F)F)cc43)nn2C2CCN(CCOCc3ncc(C(=O)O)cn3)CC2)C1]

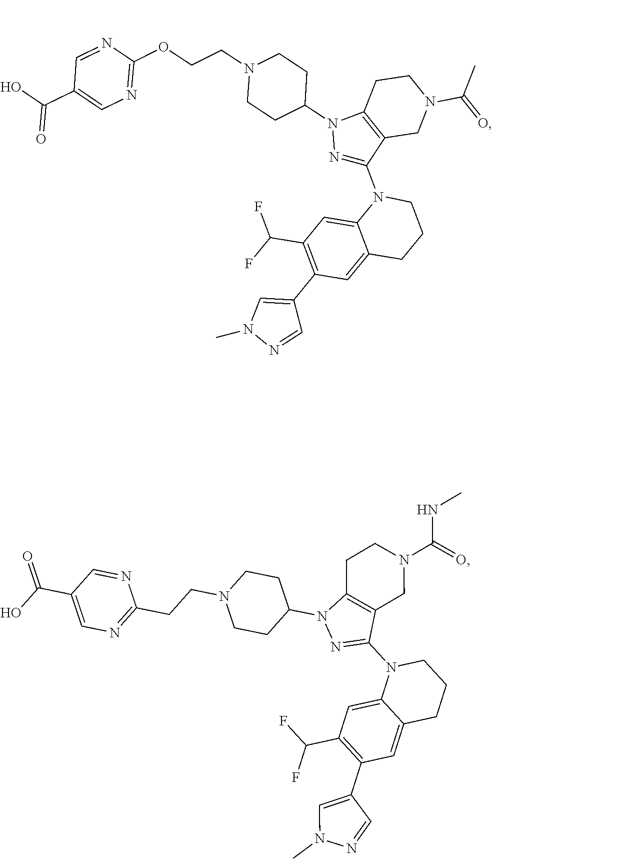 [SMILES: CC(=O)N1CCc2c(c(N3CCCc4cc(-c5cnn(C)c5)c(C(F)F)cc43)nn2C2CCN(CCOc3ncc(C(=O)O)cn3)CC2)C1 ; CNC(=O)N1CCc2c(c(N3CCCc4cc(-c5cnn(C)c5)c(C(F)F)cc43)nn2C2CCN(CCc3ncc(C(=O)O)cn3)CC2)C1]

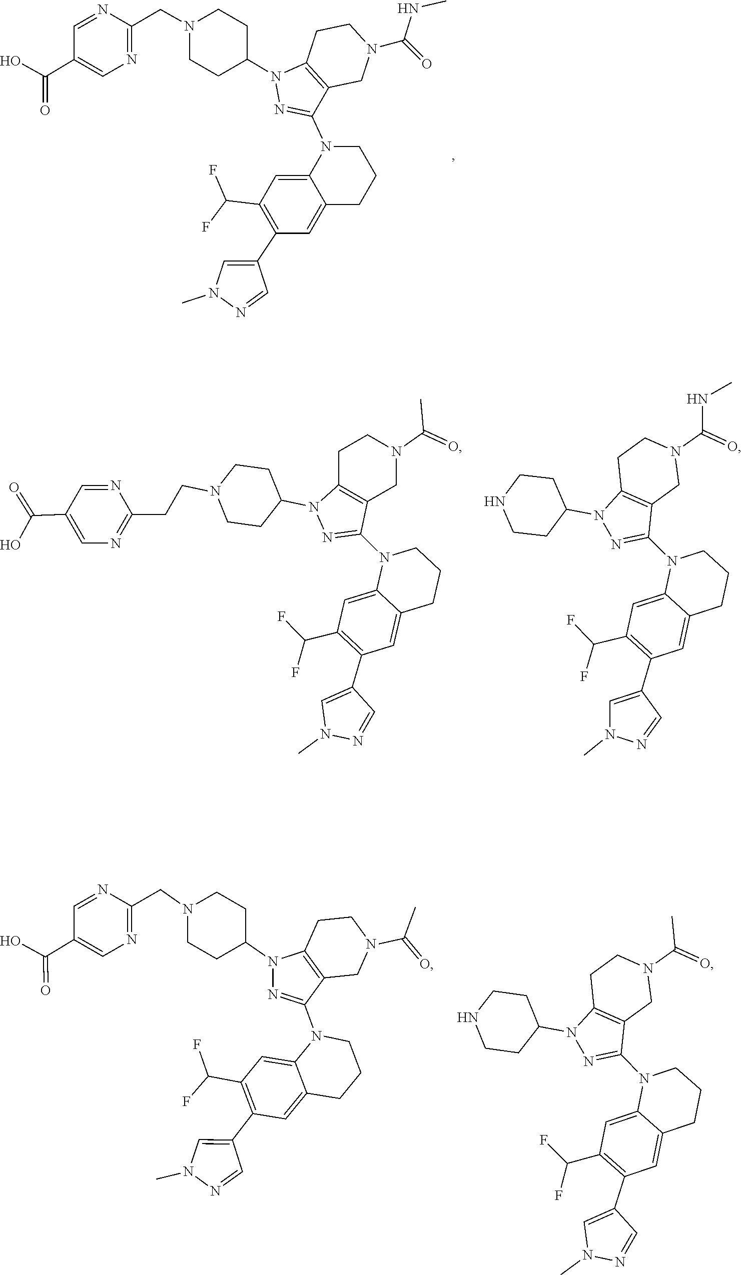 [SMILES: CNC(=O)N1CCc2c(c(N3CCCc4cc(-c5cnn(C)c5)c(C(F)F)cc43)nn2C2CCN(Cc3ncc(C(=O)O)cn3)CC2)C1 ; CC(=O)N1CCc2c(c(N3CCCc4cc(-c5cnn(C)c5)c(C(F)F)cc43)nn2C2CCN(CCc3ncc(C(=O)O)cn3)CC2)C1 ; CNC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(Cc3ncc(C(=O)O)cn3)CC2)C1 ; CC(=O)N1CCc2c(c(N3CCCc4cc(-c5cnn(C)c5)c(C(F)F)cc43)nn2C2CCNCC2)C1]

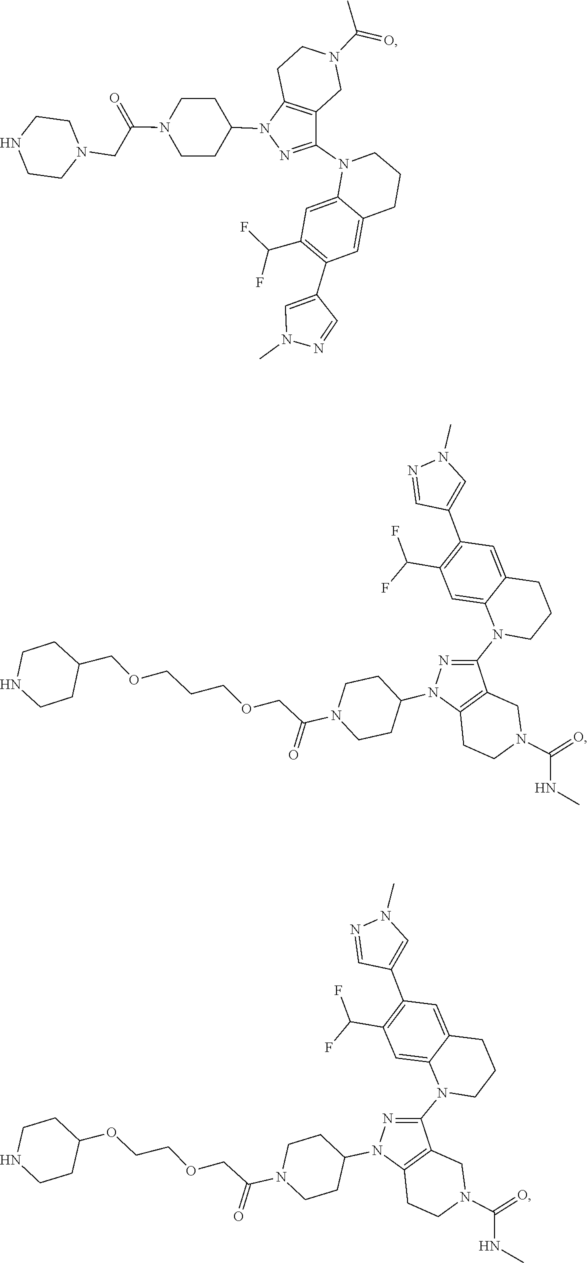 [SMILES: CC(=O)N1CCc2c(c(N3CCCc4cc(-c5cnn(C)c5)c(C(F)F)cc43)nn2C2CCN(C(=O)CN3CCNCC3)CC2)C1 ; CNC(=O)N1CCc2c(c(N3CCCc4cc(-c5cnn(C)c5)c(C(F)F)cc43)nn2C2CCN(C(=O)COCCCOCC3CCNCC3)CC2)C1 ; CNC(=O)N1CCc2c(c(N3CCCc4cc(-c5cnn(C)c5)c(C(F)F)cc43)nn2C2CCN(C(=O)COCCOC3CCNCC3)CC2)C1]

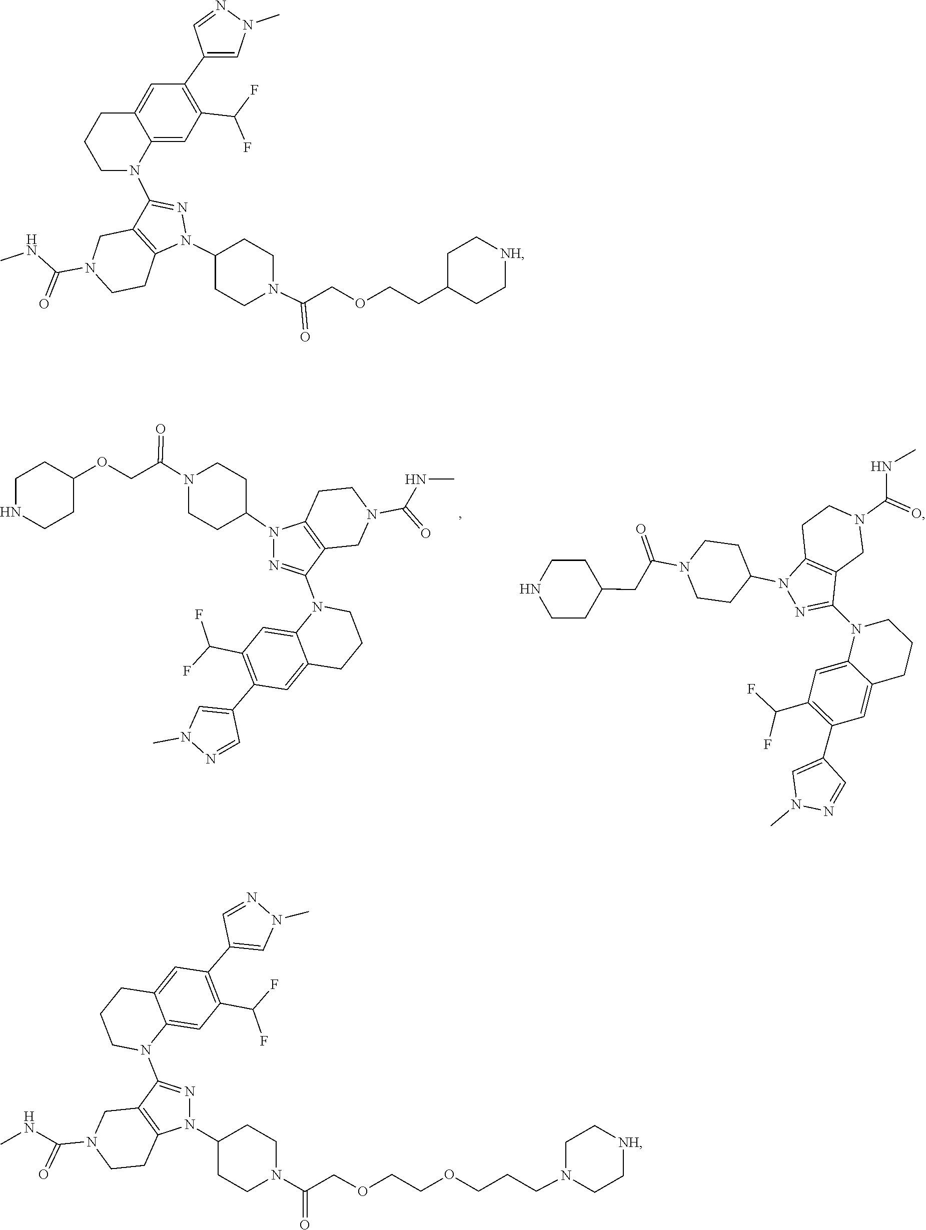 [SMILES: CNC(=O)N1CCc2c(c(N3CCCc4cc(-c5cnn(C)c5)c(C(F)F)cc43)nn2C2CCN(C(=O)COCCC3CCNCC3)CC2)C1 ; CNC(=O)N1CCc2c(c(N3CCCc4cc(-c5cnn(C)c5)c(C(F)F)cc43)nn2C2CCN(C(=O)COC3CCNCC3)CC2)C1 ; CNC(=O)N1CCc2c(c(N3CCCc4cc(-c5cnn(C)c5)c(C(F)F)cc43)nn2C2CCN(C(=O)CC3CCNCC3)CC2)C1 ; CNC(=O)N1CCc2c(c(N3CCCc4cc(-c5cnn(C)c5)c(C(F)F)cc43)nn2C2CCN(C(=O)COCCOCCCN3CCNCC3)CC2)C1]

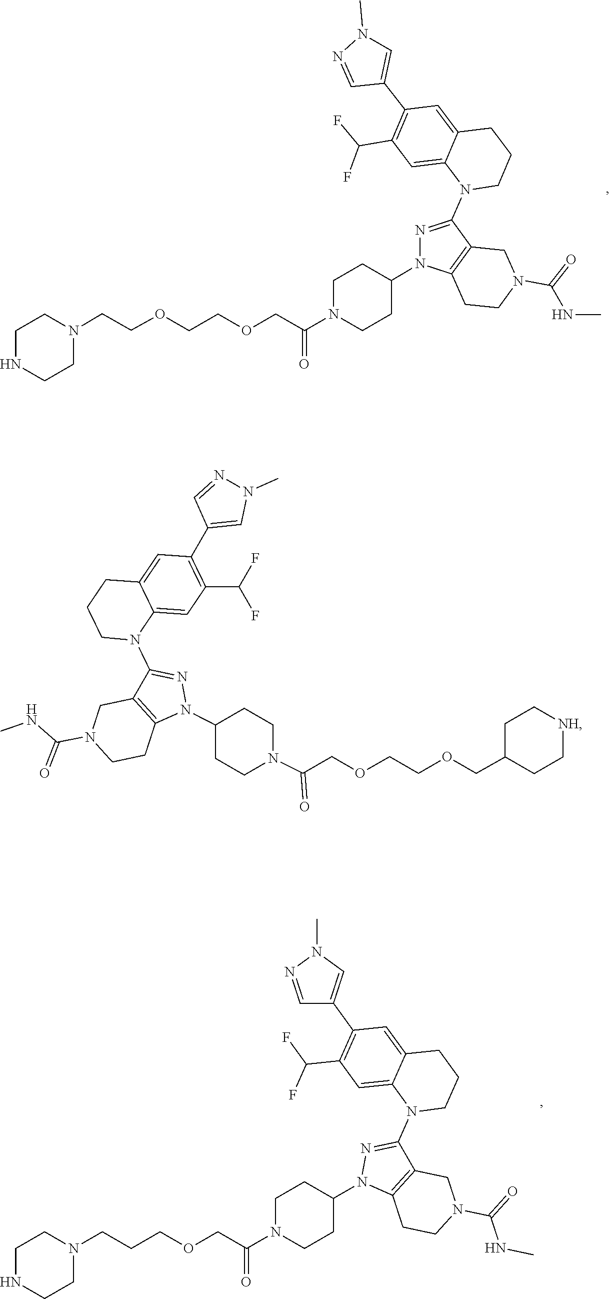

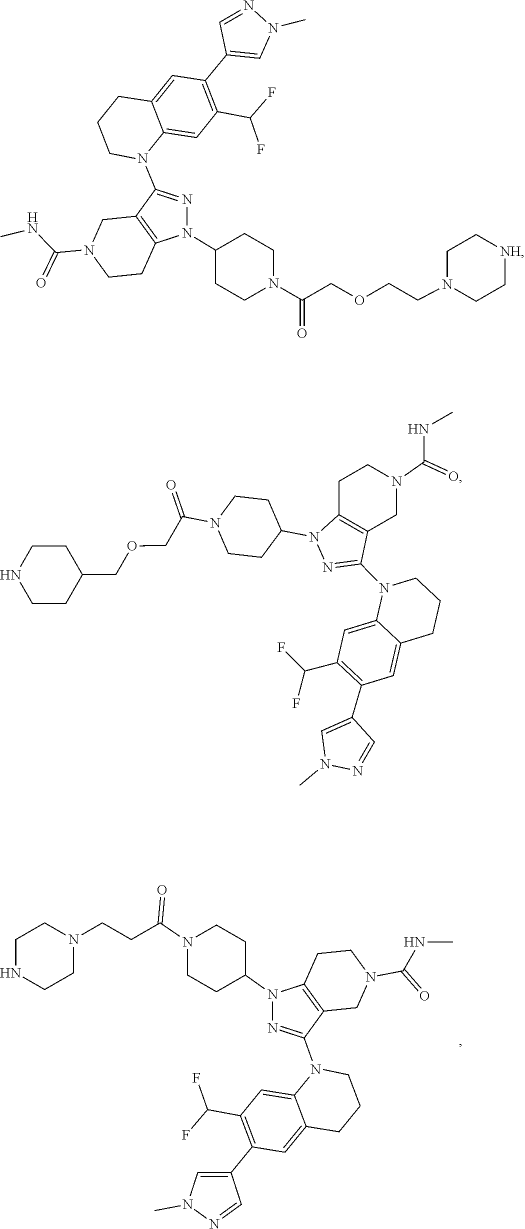 [SMILES: CNC(=O)N1CCc2c(c(N3CCCc4cc(-c5cnn(C)c5)c(C(F)F)cc43)nn2C2CCN(C(=O)COCCN3CCNCC3)CC2)C1 ; CNC(=O)N1CCc2c(c(N3CCCc4cc(-c5cnn(C)c5)c(C(F)F)cc43)nn2C2CCN(C(=O)COCC3CCNCC3)CC2)C1 ; CNC(=O)N1CCc2c(c(N3CCCc4cc(-c5cnn(C)c5)c(C(F)F)cc43)nn2C2CCN(C(=O)CCN3CCNCC3)CC2)C1]

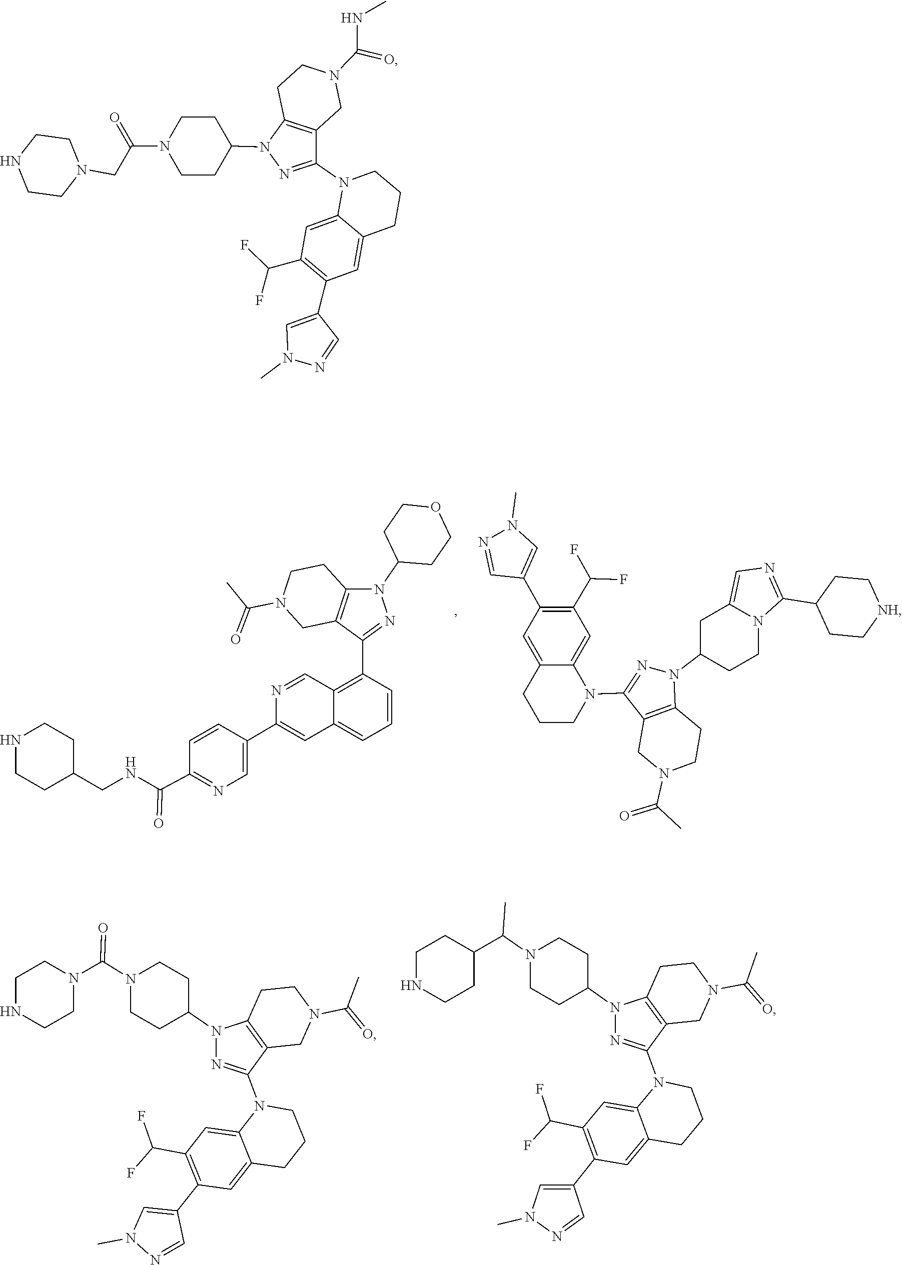

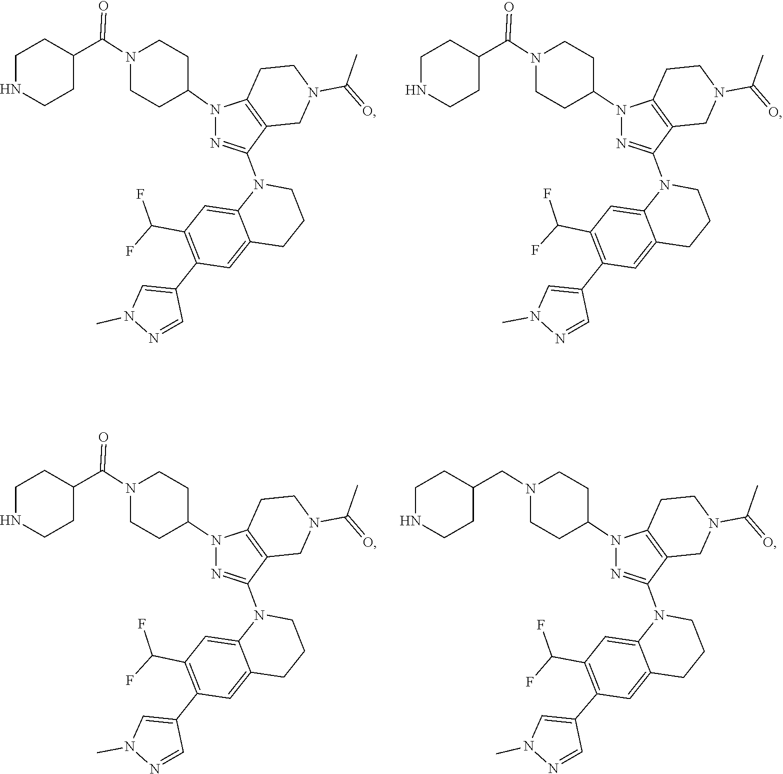 [SMILES: CC(=O)N1CCc2c(c(N3CCCc4cc(-c5cnn(C)c5)c(C(F)F)cc43)nn2C2CCN(C(=O)C3CCNCC3)CC2)C1 ; CC(=O)N1CCc2c(c(N3CCCc4cc(-c5cnn(C)c5)c(C(F)F)cc43)nn2C2CCN(C(=O)C3CCNCC3)CC2)C1 ; CC(=O)N1CCc2c(c(N3CCCc4cc(-c5cnn(C)c5)c(C(F)F)cc43)nn2C2CCN(C(=O)C3CCNCC3)CC2)C1 ; CC(=O)N1CCc2c(c(N3CCCc4cc(-c5cnn(C)c5)c(C(F)F)cc43)nn2C2CCN(CC3CCNCC3)CC2)C1]

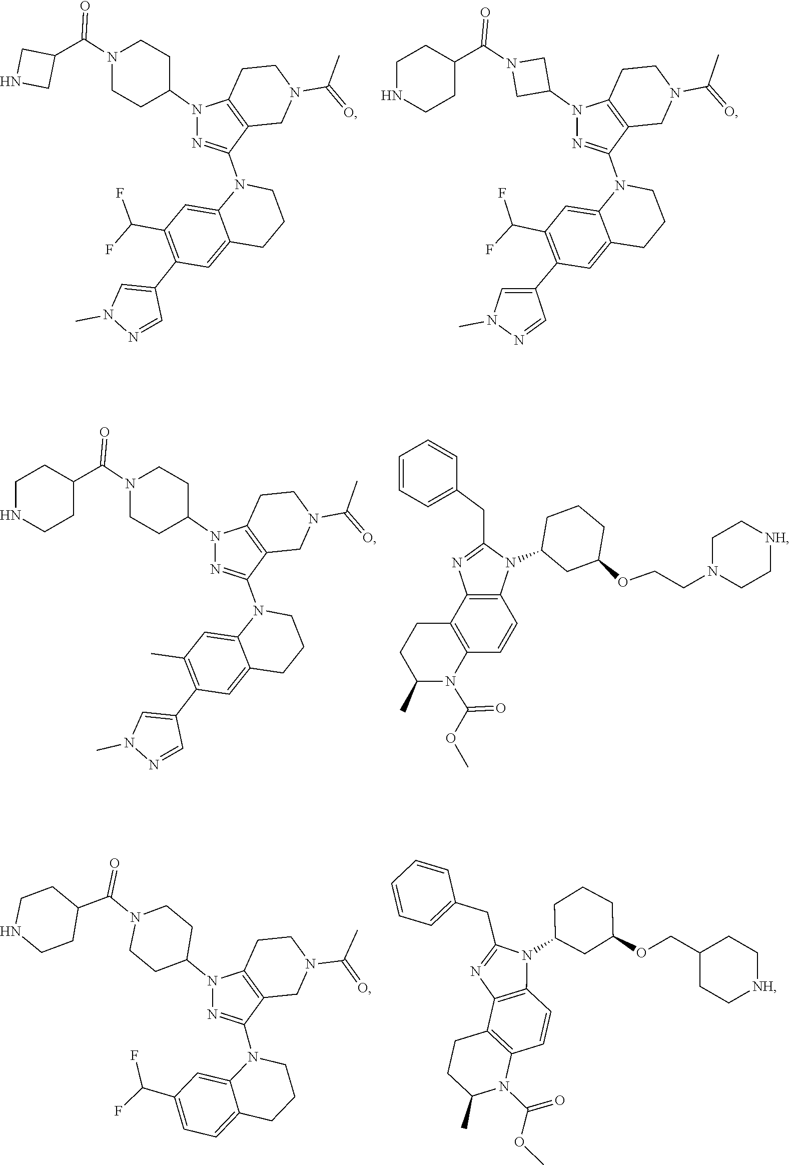

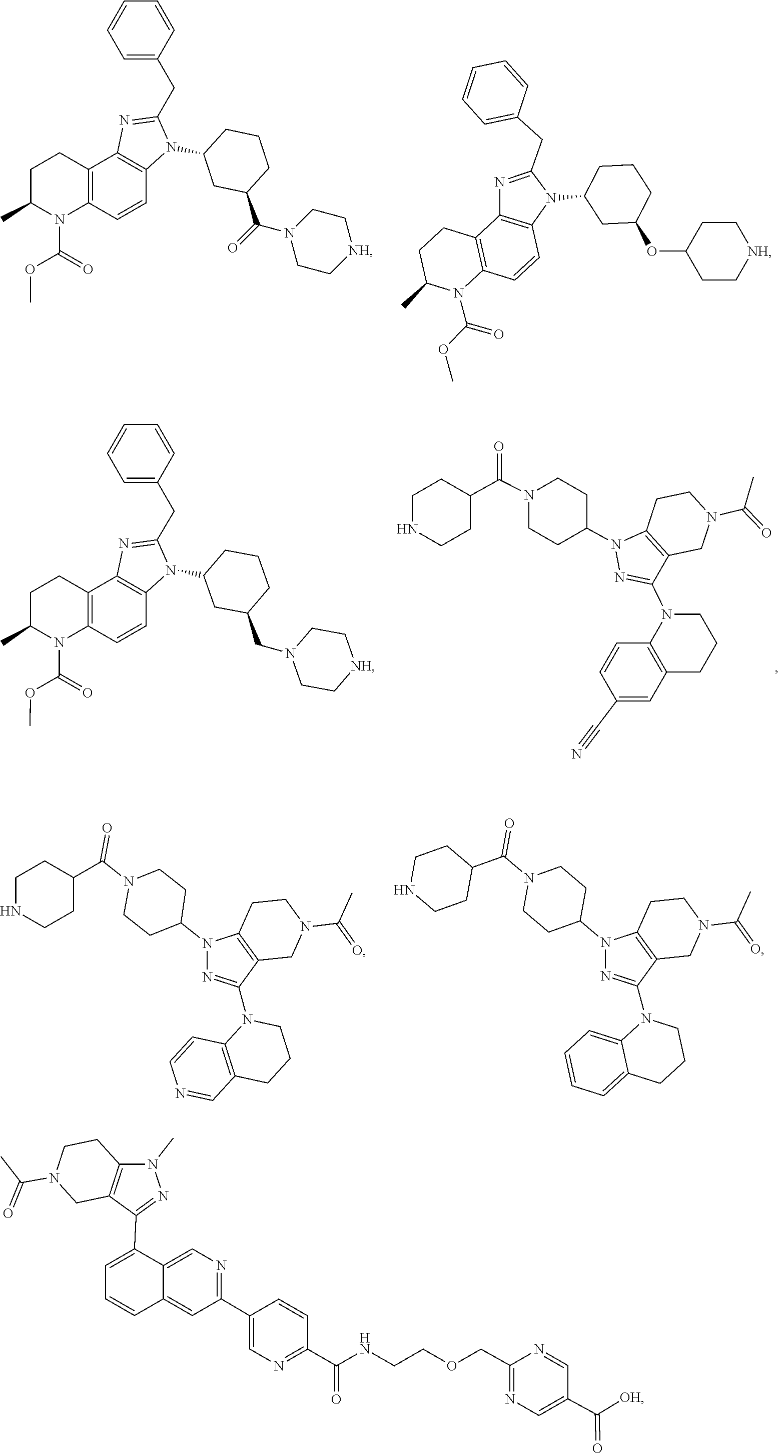

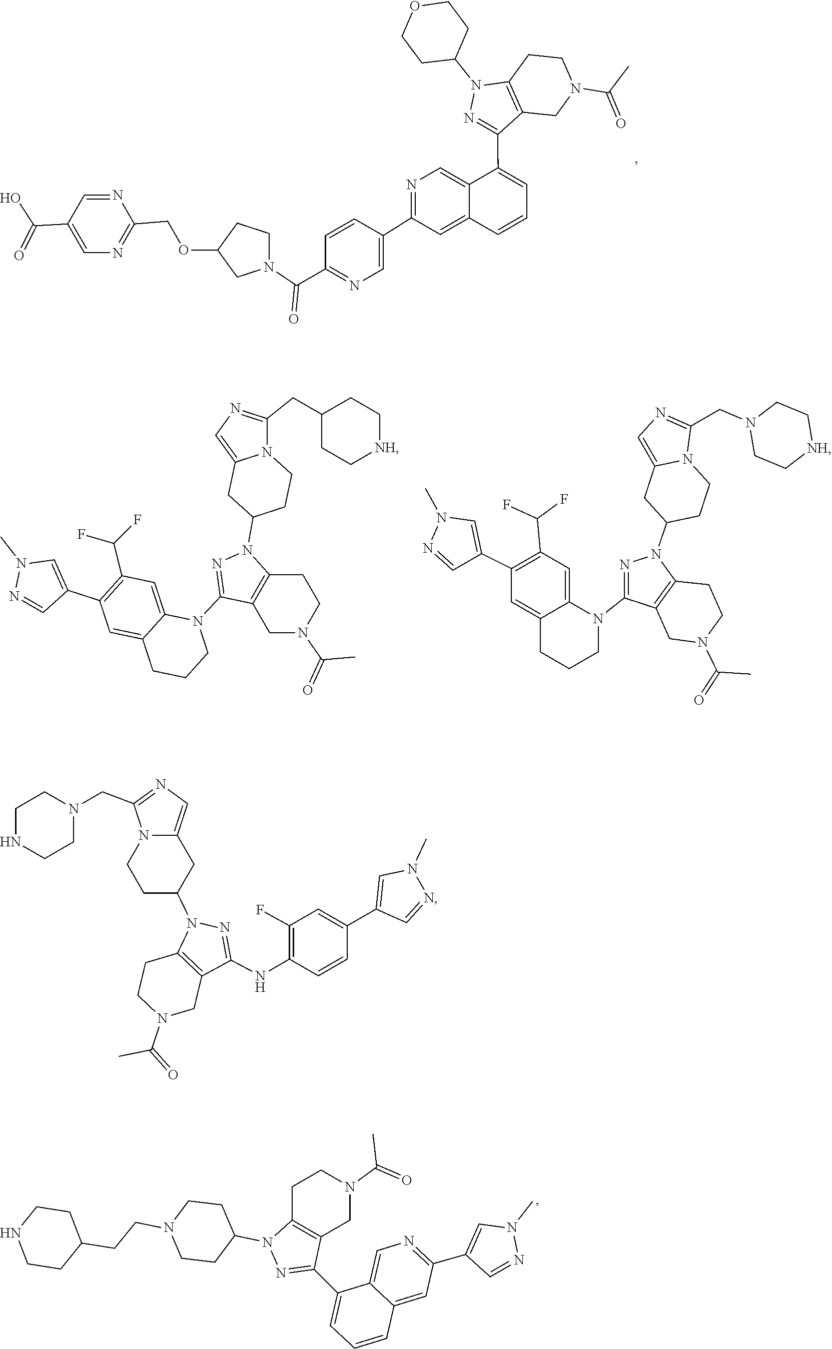

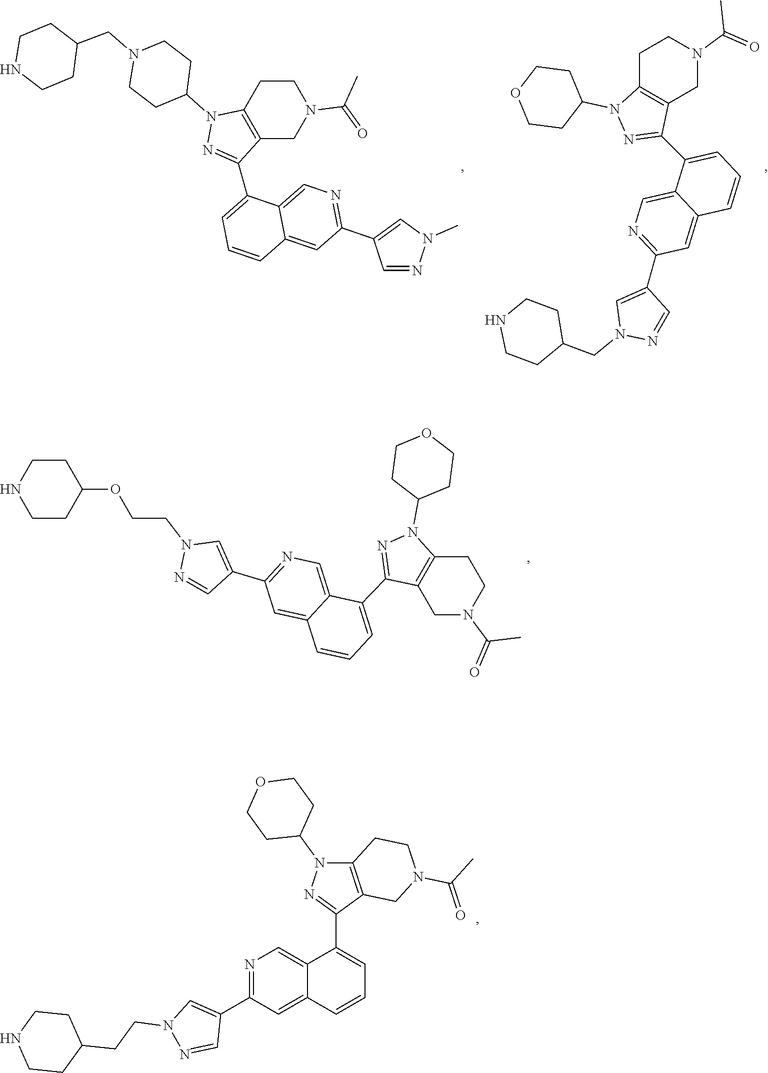 [SMILES: CC(=O)N1CCc2c(c(-c3cccc4cc(-c5cnn(C)c5)ncc34)nn2C2CCN(CC3CCNCC3)CC2)C1 ; CC(=O)N1CCc2c(c(-c3cccc4cc(-c5cnn(CC6CCNCC6)c5)ncc34)nn2C2CCOCC2)C1 ; CC(=O)N1CCc2c(c(-c3cccc4cc(-c5cnn(CCOC6CCNCC6)c5)ncc34)nn2C2CCOCC2)C1 ; CC(=O)N1CCc2c(c(-c3cccc4cc(-c5cnn(CCC6CCNCC6)c5)ncc34)nn2C2CCOCC2)C1]

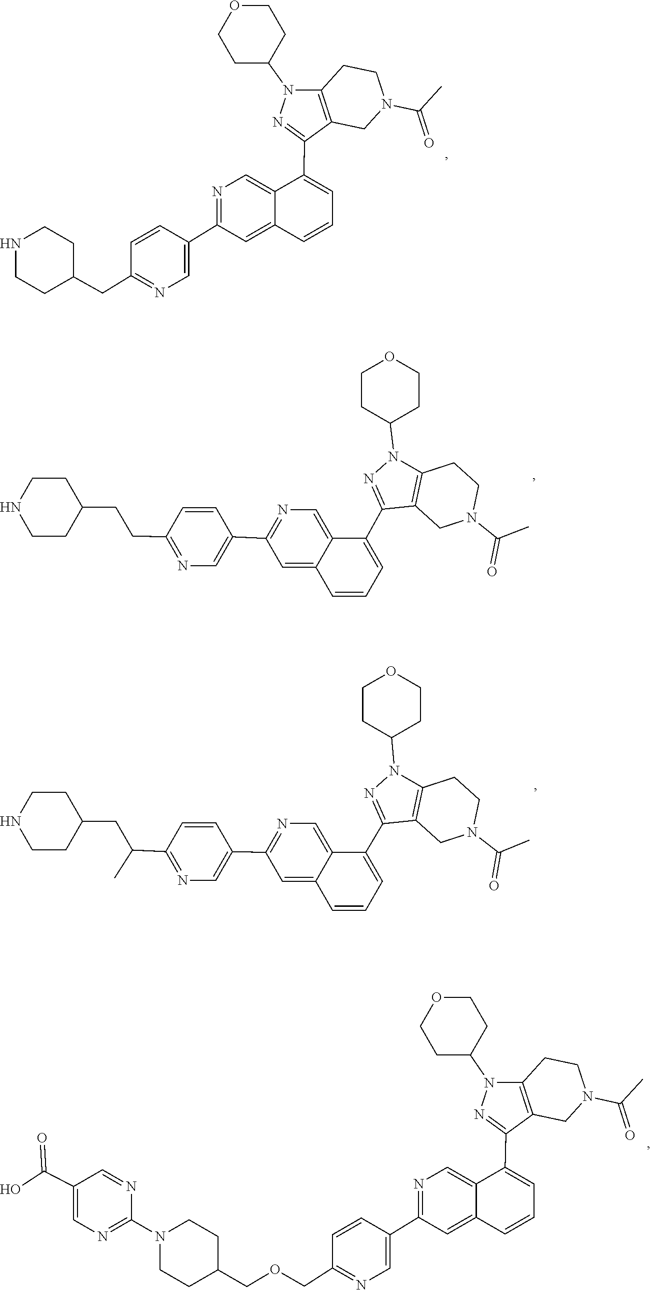 [SMILES: CC(=O)N1CCc2c(c(-c3cccc4cc(-c5ccc(CC6CCNCC6)nc5)ncc34)nn2C2CCOCC2)C1 ; CC(=O)N1CCc2c(c(-c3cccc4cc(-c5ccc(CCC6CCNCC6)nc5)ncc34)nn2C2CCOCC2)C1 ; CC(=O)N1CCc2c(c(-c3cccc4cc(-c5ccc(C(C)CC6CCNCC6)nc5)ncc34)nn2C2CCOCC2)C1 ; CC(=O)N1CCc2c(c(-c3cccc4cc(-c5ccc(COCC6CCN(c7ncc(C(=O)O)cn7)CC6)nc5)ncc34)nn2C2CCOCC2)C1]

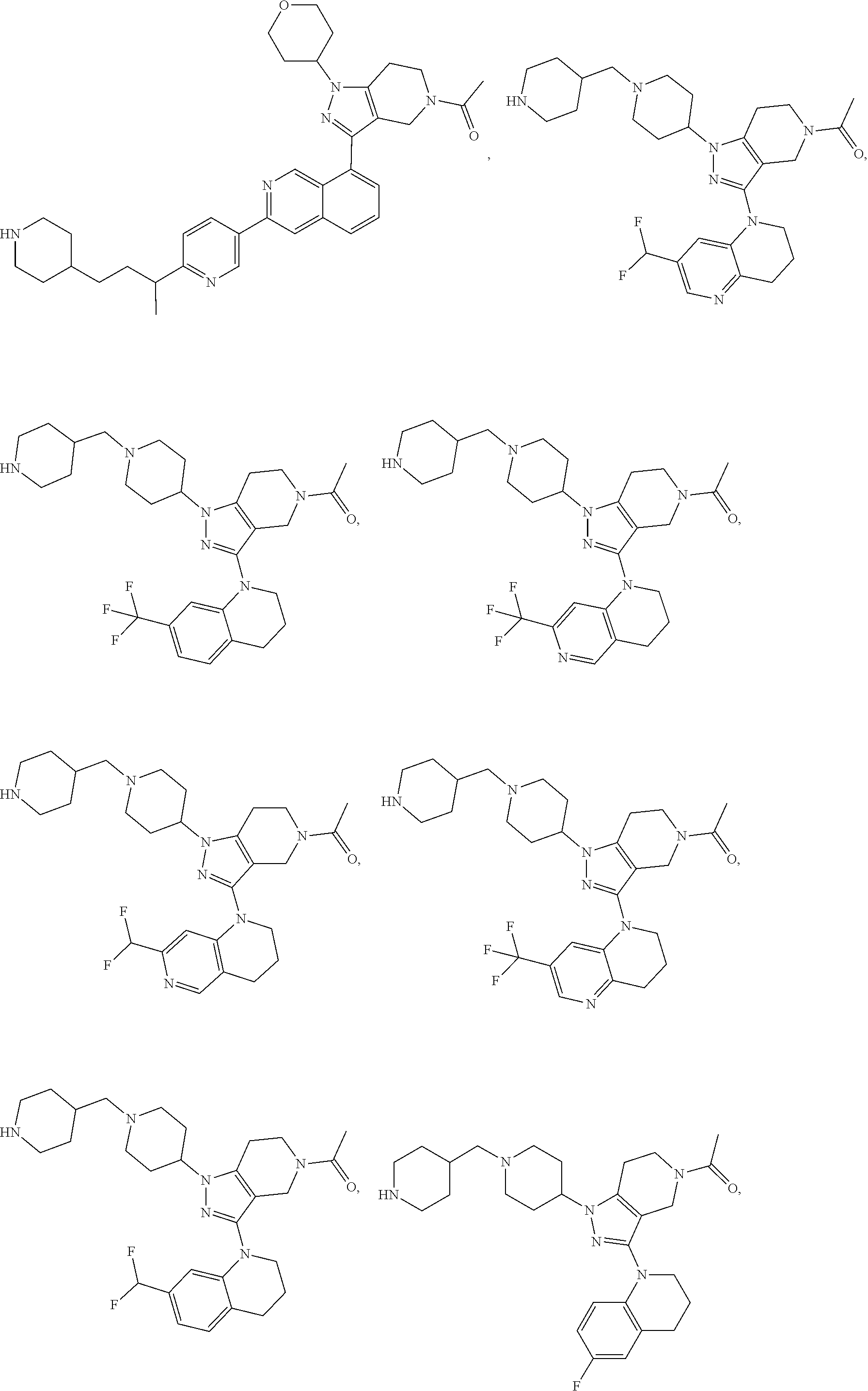

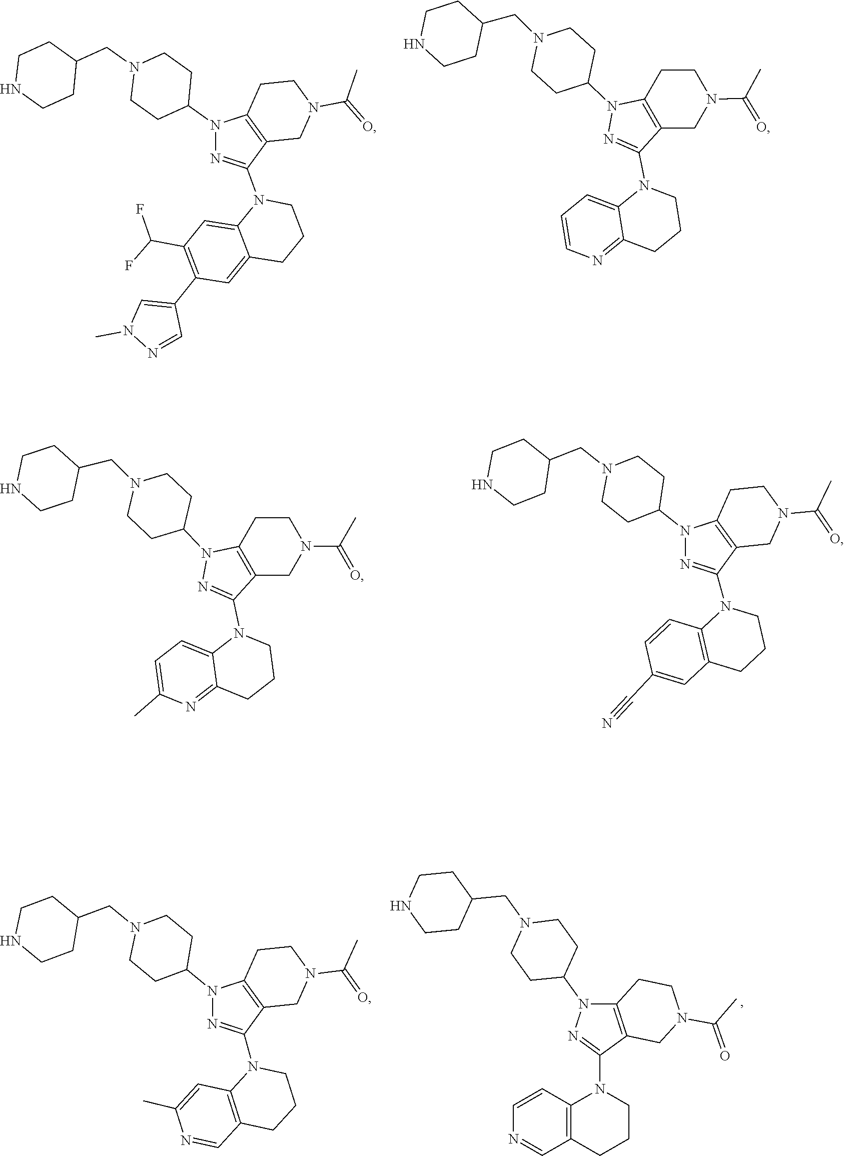 [SMILES: CC(=O)N1CCc2c(c(N3CCCc4cc(-c5cnn(C)c5)c(C(F)F)cc43)nn2C2CCN(CC3CCNCC3)CC2)C1 ; CC(=O)N1CCc2c(c(N3CCCc4ncccc43)nn2C2CCN(CC3CCNCC3)CC2)C1 ; CC(=O)N1CCc2c(c(N3CCCc4nc(C)ccc43)nn2C2CCN(CC3CCNCC3)CC2)C1 ; CC(=O)N1CCc2c(c(N3CCCc4cc(C#N)ccc43)nn2C2CCN(CC3CCNCC3)CC2)C1 ; CC(=O)N1CCc2c(c(N3CCCc4cnc(C)cc43)nn2C2CCN(CC3CCNCC3)CC2)C1 ; CC(=O)N1CCc2c(c(N3CCCc4cnccc43)nn2C2CCN(CC3CCNCC3)CC2)C1]

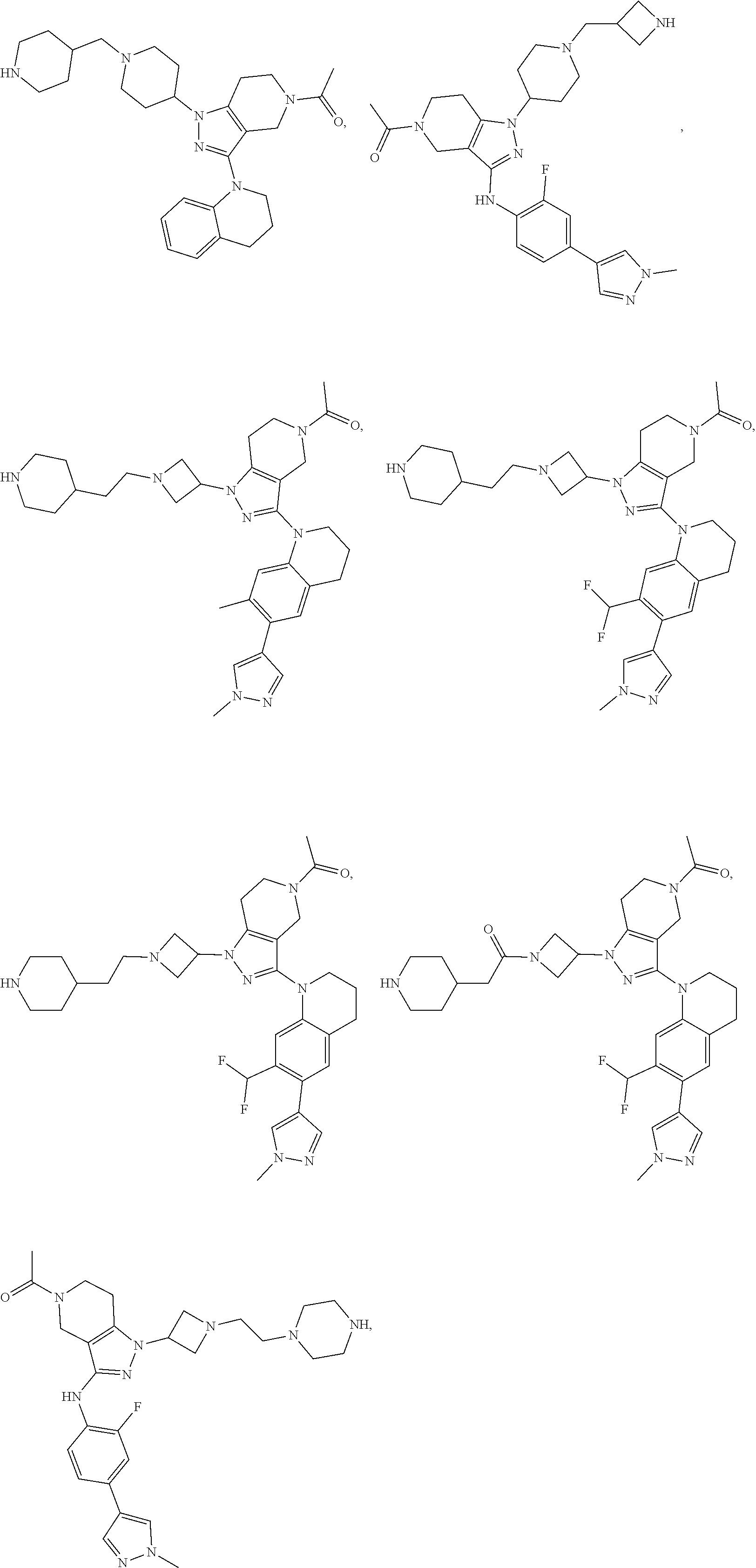

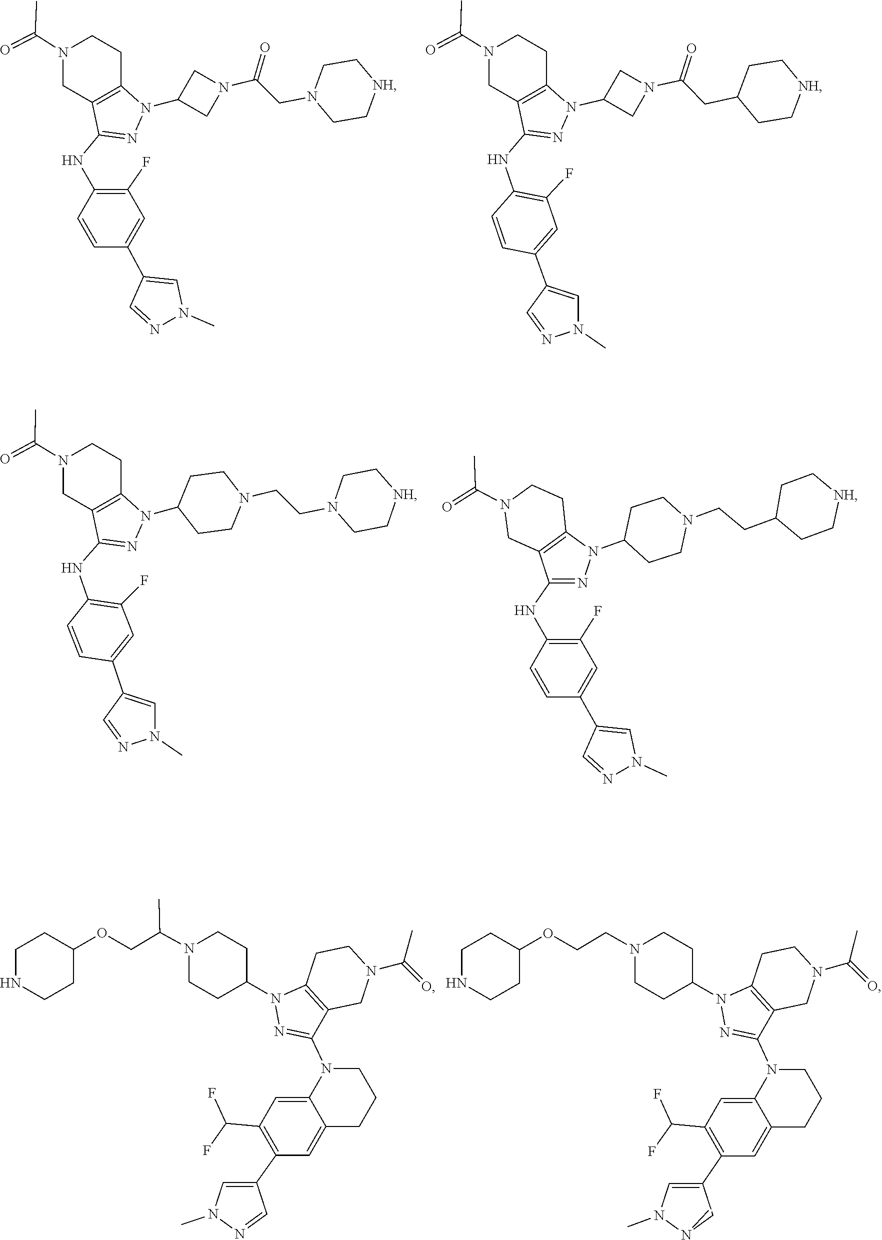

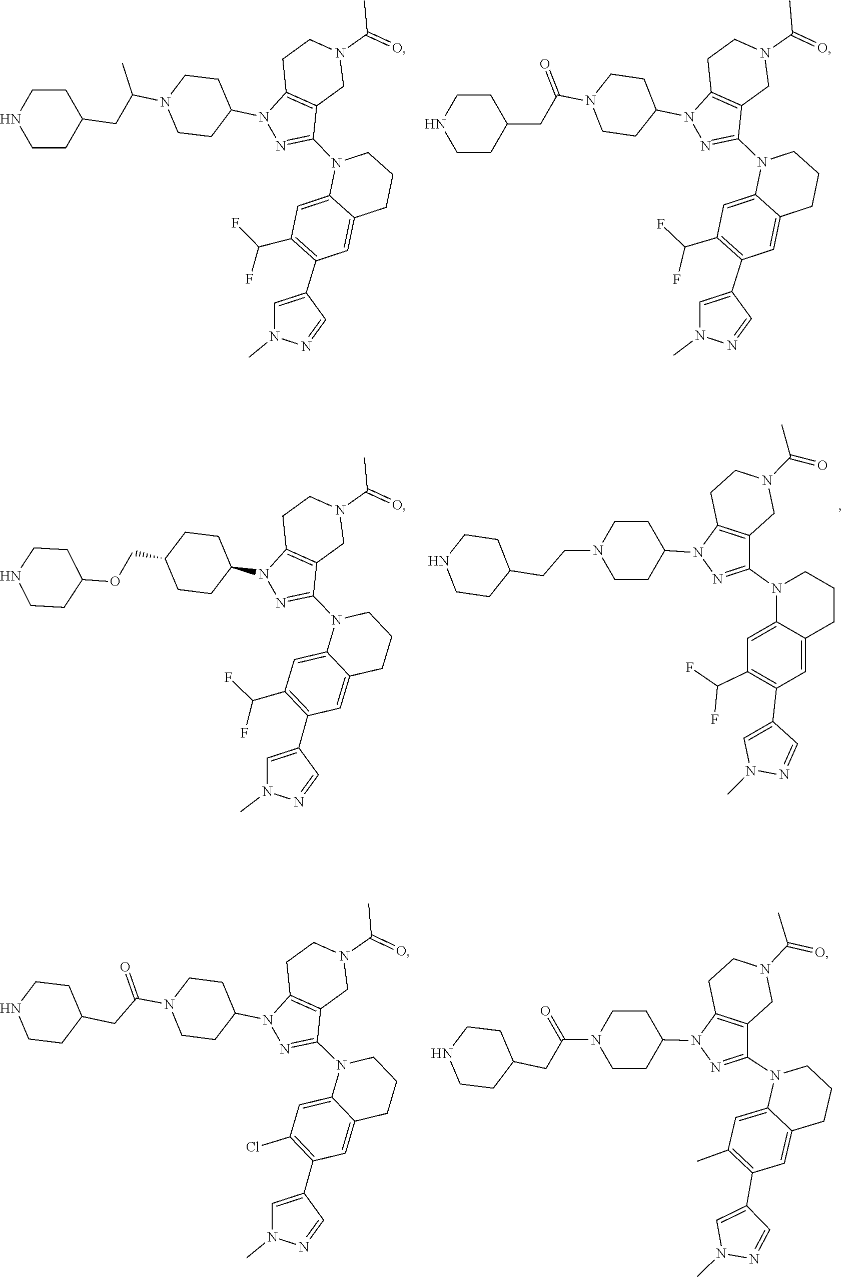 [SMILES: CC(=O)N1CCc2c(c(N3CCCc4cc(-c5cnn(C)c5)c(C(F)F)cc43)nn2C2CCN(C(C)CC3CCNCC3)CC2)C1 ; CC(=O)N1CCc2c(c(N3CCCc4cc(-c5cnn(C)c5)c(C(F)F)cc43)nn2C2CCN(C(=O)CC3CCNCC3)CC2)C1 ; CC(=O)N1CCc2c(c(N3CCCc4cc(-c5cnn(C)c5)c(C(F)F)cc43)nn2[C@H]2CC[C@H](COC3CCNCC3)CC2)C1 ; CC(=O)N1CCc2c(c(N3CCCc4cc(-c5cnn(C)c5)c(C(F)F)cc43)nn2C2CCN(CCC3CCNCC3)CC2)C1 ; CC(=O)N1CCc2c(c(N3CCCc4cc(-c5cnn(C)c5)c(Cl)cc43)nn2C2CCN(C(=O)CC3CCNCC3)CC2)C1 ; CC(=O)N1CCc2c(c(N3CCCc4cc(-c5cnn(C)c5)c(C)cc43)nn2C2CCN(C(=O)CC3CCNCC3)CC2)C1]

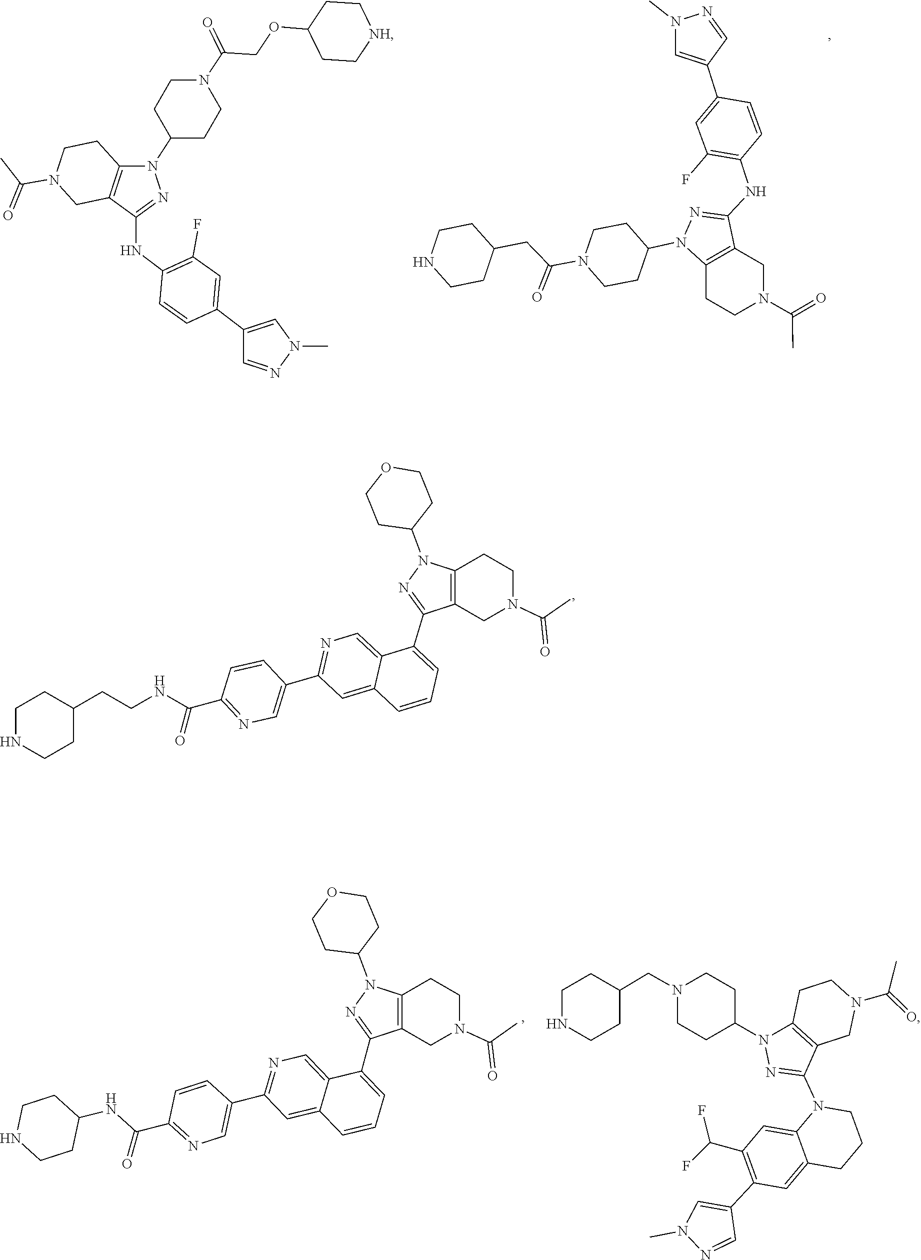

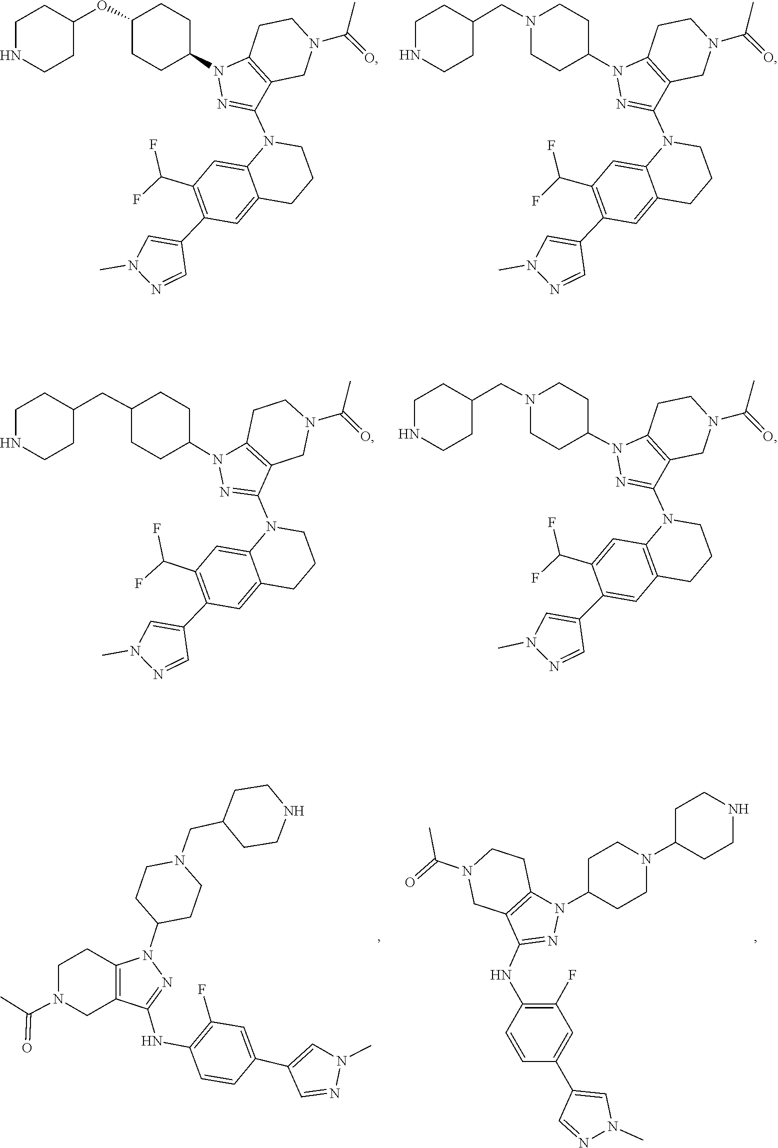

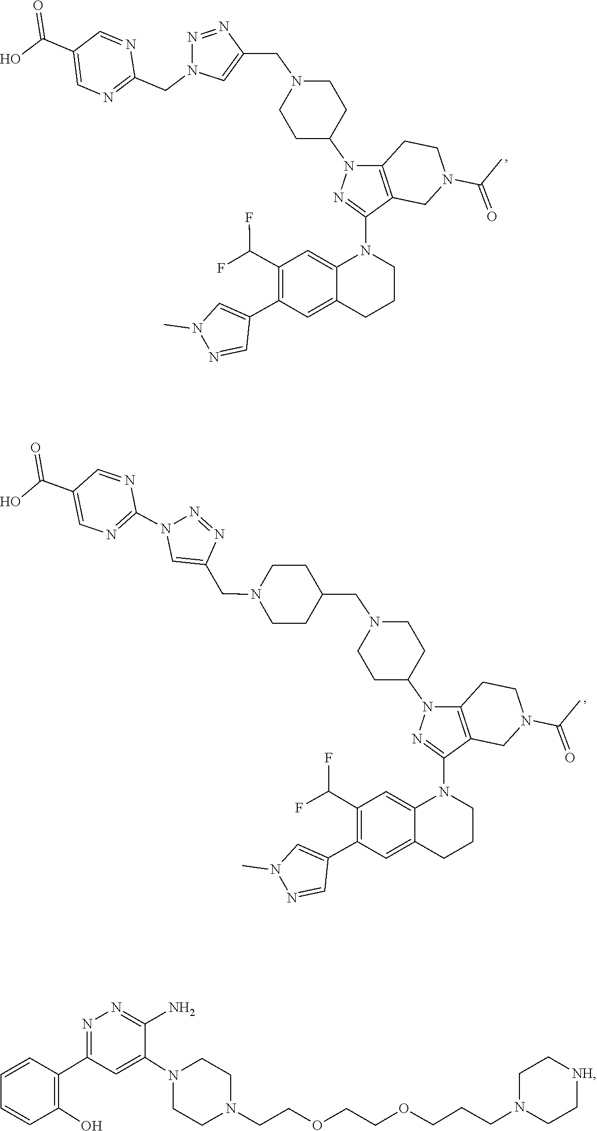 [SMILES: CC(=O)N1CCc2c(c(N3CCCc4cc(-c5cnn(C)c5)c(C(F)F)cc43)nn2C2CCN(Cc3cn(Cc4ncc(C(=O)O)cn4)nn3)CC2)C1 ; CC(=O)N1CCc2c(c(N3CCCc4cc(-c5cnn(C)c5)c(C(F)F)cc43)nn2C2CCN(CC3CCN(Cc4cn(-c5ncc(C(=O)O)cn5)nn4)CC3)CC2)C1 ; Nc1nnc(-c2ccccc2O)cc1N1CCN(CCOCCOCCCN2CCNCC2)CC1]

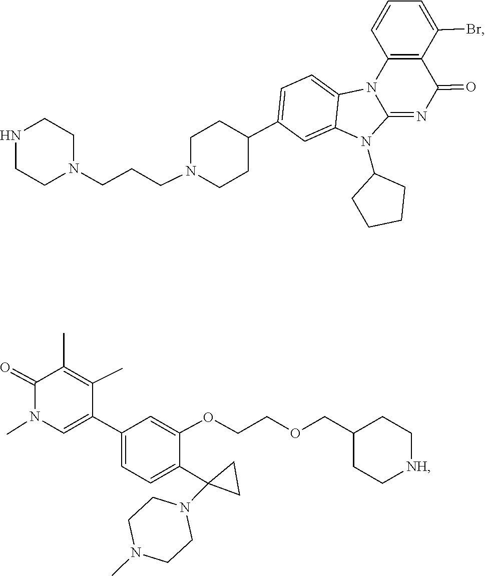 [SMILES: O=c1nc2n(C3CCCC3)c3cc(C4CCN(CCCN5CCNCC5)CC4)ccc3n2c2cccc(Br)c12 ; Cc1c(-c2ccc(C3(N4CCN(C)CC4)CC3)c(OCCOCC3CCNCC3)c2)cn(C)c(=O)c1C]

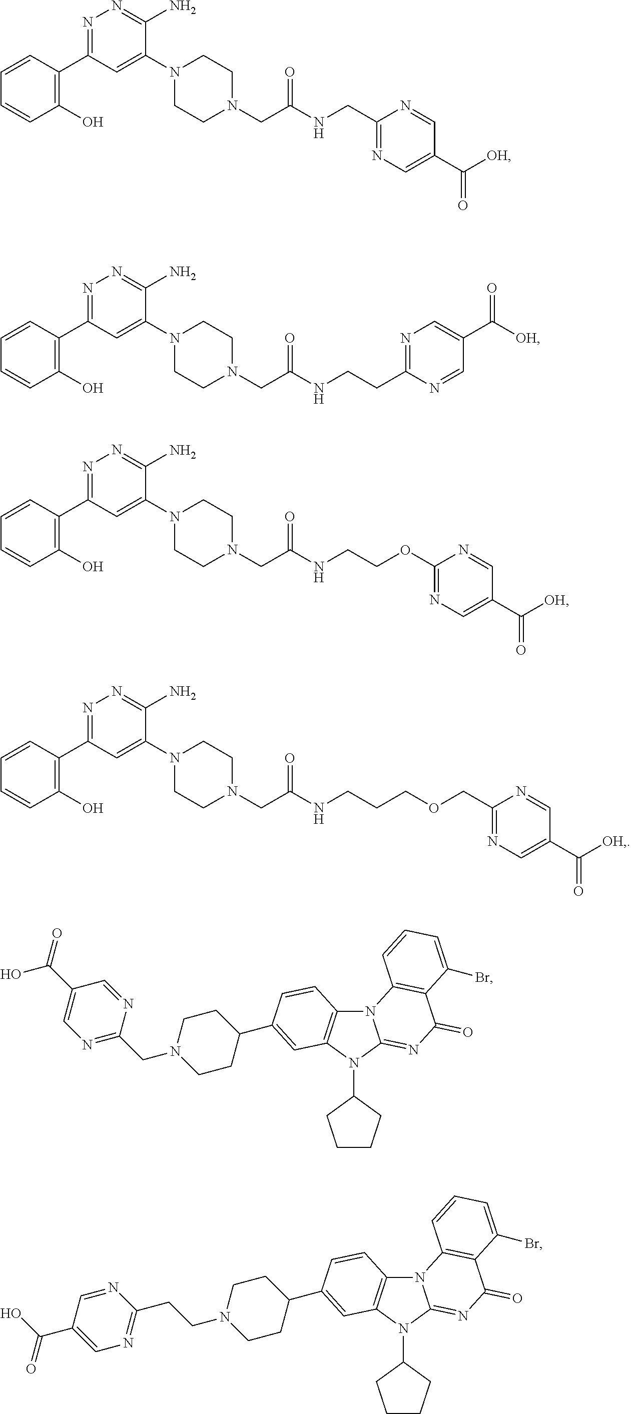 [SMILES: Nc1nnc(-c2ccccc2O)cc1N1CCN(CC(=O)NCc2ncc(C(=O)O)cn2)CC1 ; Nc1nnc(-c2ccccc2O)cc1N1CCN(CC(=O)NCCc2ncc(C(=O)O)cn2)CC1 ; Nc1nnc(-c2ccccc2O)cc1N1CCN(CC(=O)NCCOc2ncc(C(=O)O)cn2)CC1 ; Nc1nnc(-c2ccccc2O)cc1N1CCN(CC(=O)NCCCOCc2ncc(C(=O)O)cn2)CC1 ; O=C(O)c1cnc(CN2CCC(c3ccc4c(c3)n(C3CCCC3)c3nc(=O)c5c(Br)cccc5n43)CC2)nc1 ; O=C(O)c1cnc(CCN2CCC(c3ccc4c(c3)n(C3CCCC3)c3nc(=O)c5c(Br)cccc5n43)CC2)nc1]

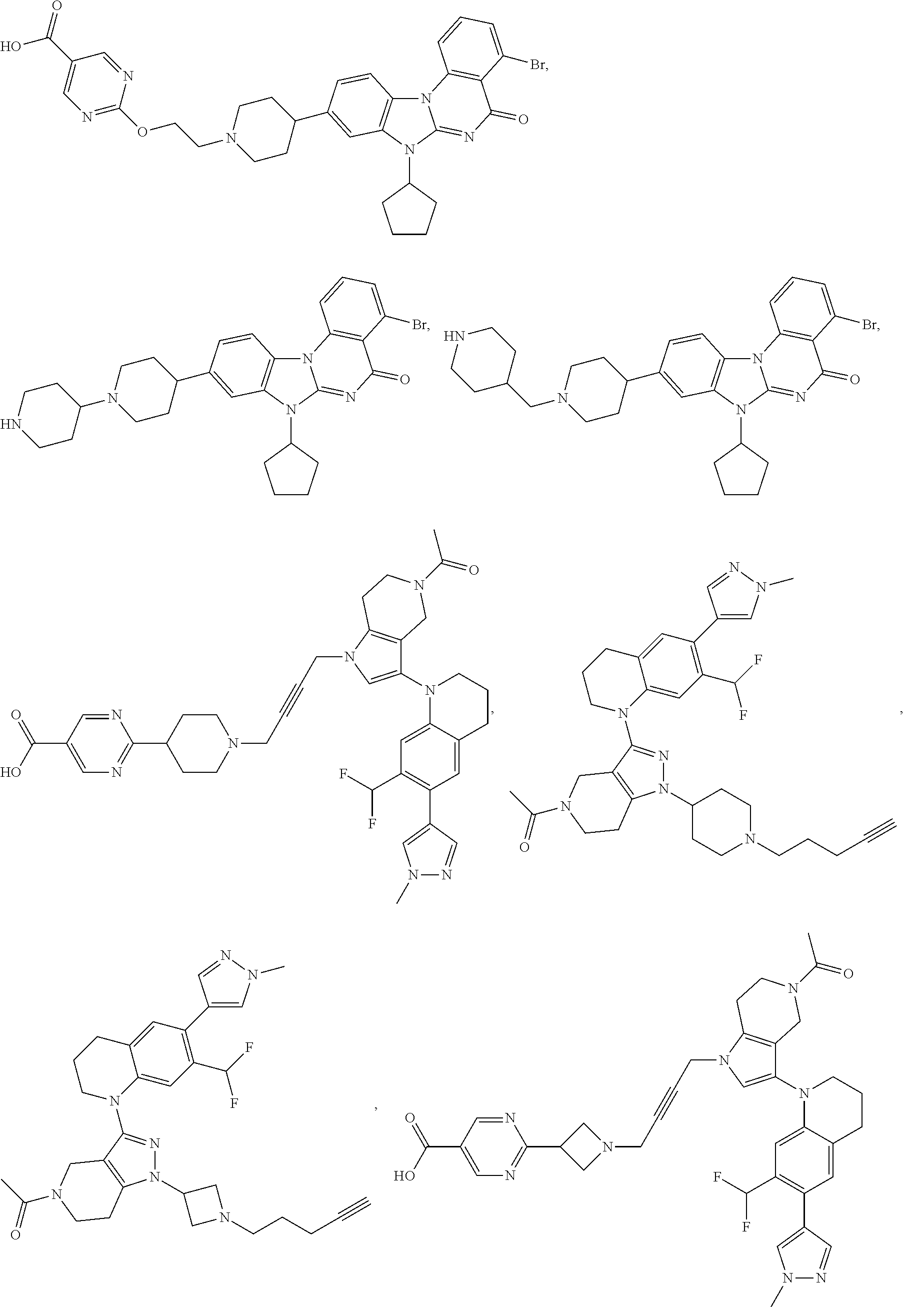

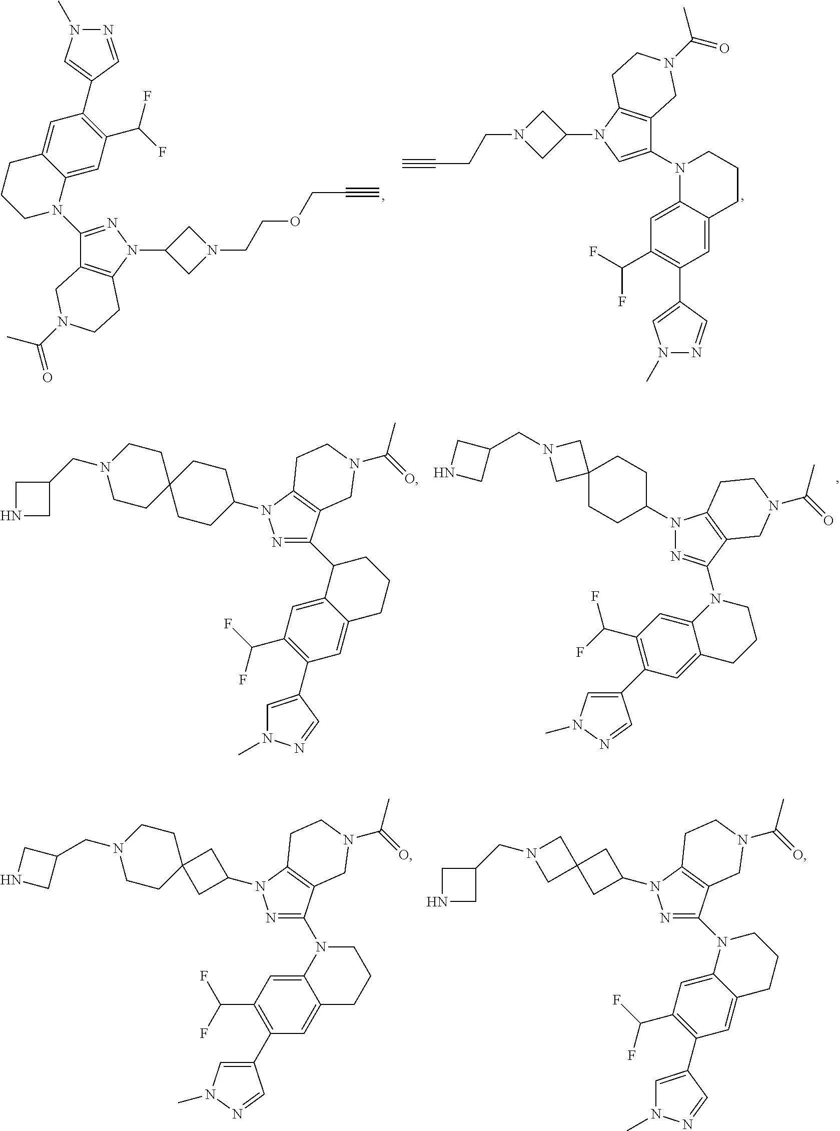

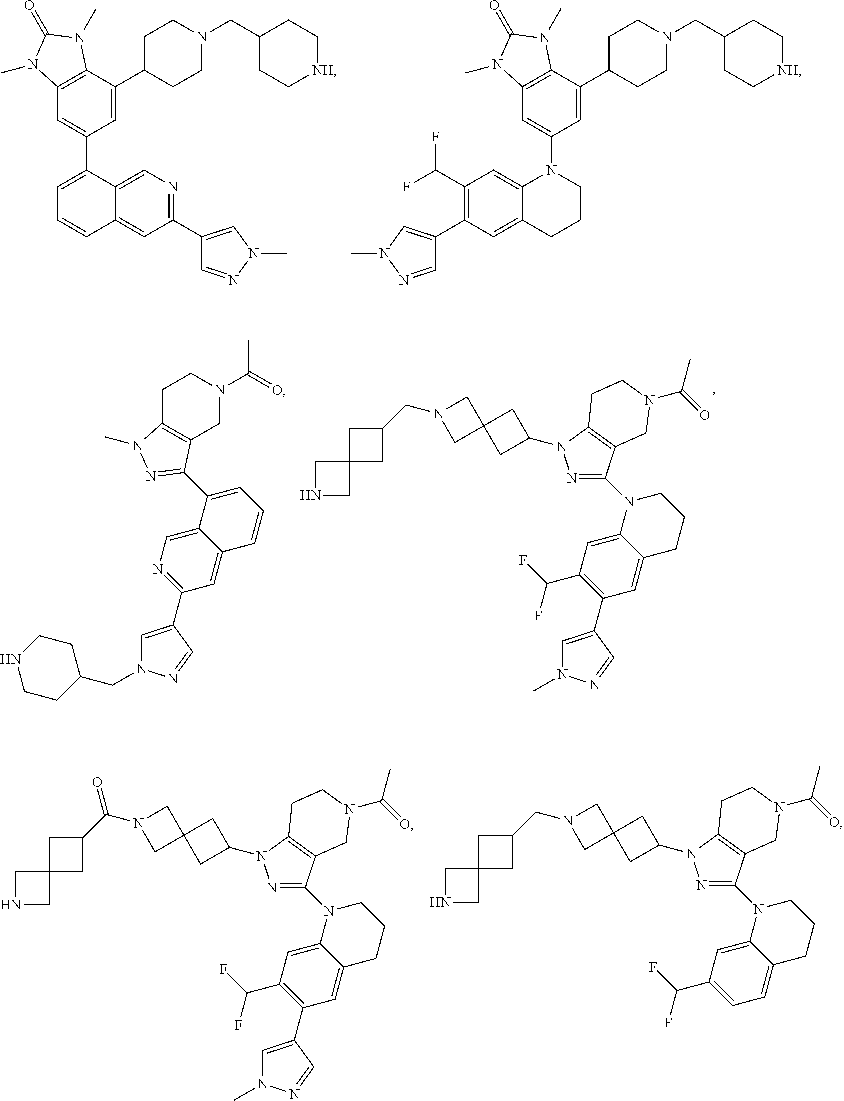 [SMILES: Cn1cc(-c2cc3cccc(-c4cc(C5CCN(CC6CCNCC6)CC5)c5c(c4)n(C)c(=O)n5C)c3cn2)cn1 ; Cn1cc(-c2cc3c(cc2C(F)F)N(c2cc(C4CCN(CC5CCNCC5)CC4)c4c(c2)n(C)c(=O)n4C)CCC3)cn1 ; CC(=O)N1CCc2c(c(-c3cccc4cc(-c5cnn(CC6CCNCC6)c5)ncc34)nn2C)C1 ; CC(=O)N1CCc2c(c(N3CCCc4cc(-c5cnn(C)c5)c(C(F)F)cc43)nn2C2CC3(C2)CN(CC2CC4(CNC4)C2)C3)C1 ; CC(=O)N1CCc2c(c(N3CCCc4cc(-c5cnn(C)c5)c(C(F)F)cc43)nn2C2CC3(C2)CN(C(=O)C2CC4(CNC4)C2)C3)C1 ; CC(=O)N1CCc2c(c(N3CCCc4ccc(C(F)F)cc43)nn2C2CC3(C2)CN(CC2CC4(CNC4)C2)C3)C1]

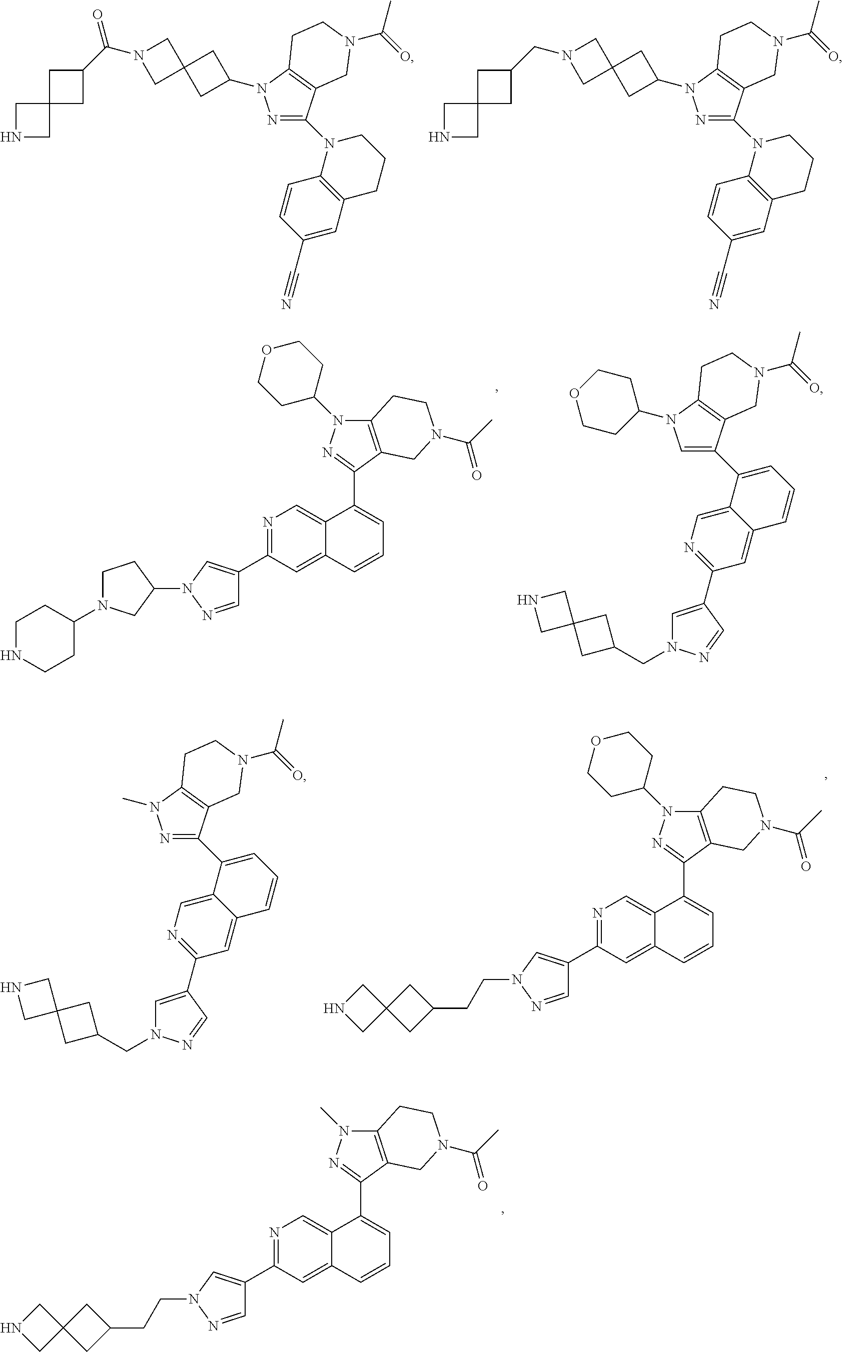

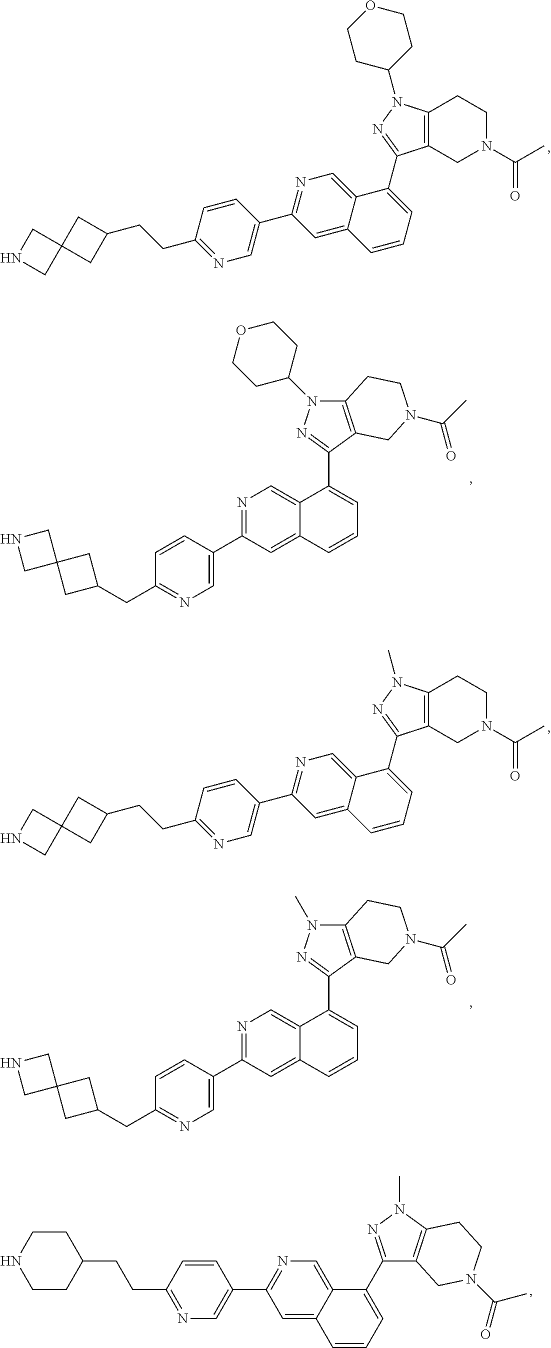 [SMILES: CC(=O)N1CCc2c(c(-c3cccc4cc(-c5ccc(CCC6CC7(CNC7)C6)nc5)ncc34)nn2C2CCOCC2)C1 ; CC(=O)N1CCc2c(c(-c3cccc4cc(-c5ccc(CC6CC7(CNC7)C6)nc5)ncc34)nn2C2CCOCC2)C1 ; CC(=O)N1CCc2c(c(-c3cccc4cc(-c5ccc(CCC6CC7(CNC7)C6)nc5)ncc34)nn2C)C1 ; CC(=O)N1CCc2c(c(-c3cccc4cc(-c5ccc(CC6CC7(CNC7)C6)nc5)ncc34)nn2C)C1 ; CC(=O)N1CCc2c(c(-c3cccc4cc(-c5ccc(CCC6CCNCC6)nc5)ncc34)nn2C)C1]

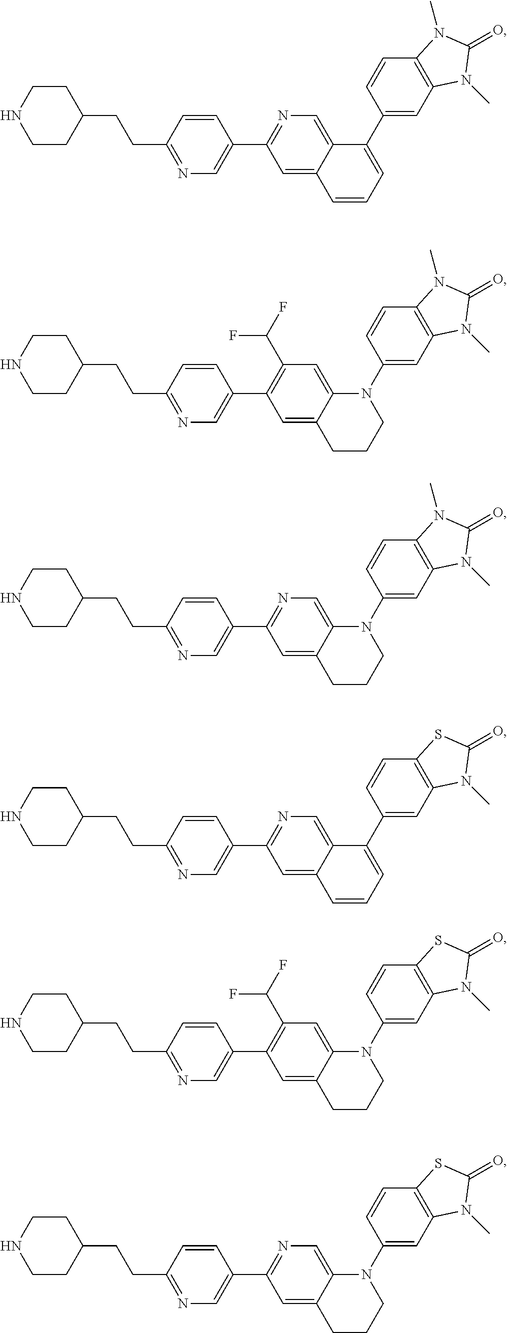 [SMILES: Cn1c(=O)n(C)c2cc(-c3cccc4cc(-c5ccc(CCC6CCNCC6)nc5)ncc34)ccc21 ; Cn1c(=O)n(C)c2cc(N3CCCc4cc(-c5ccc(CCC6CCNCC6)nc5)c(C(F)F)cc43)ccc21 ; Cn1c(=O)n(C)c2cc(N3CCCc4cc(-c5ccc(CCC6CCNCC6)nc5)ncc43)ccc21 ; Cn1c(=O)sc2ccc(-c3cccc4cc(-c5ccc(CCC6CCNCC6)nc5)ncc34)cc21 ; Cn1c(=O)sc2ccc(N3CCCc4cc(-c5ccc(CCC6CCNCC6)nc5)c(C(F)F)cc43)cc21 ; Cn1c(=O)sc2ccc(N3CCCc4cc(-c5ccc(CCC6CCNCC6)nc5)ncc43)cc21]

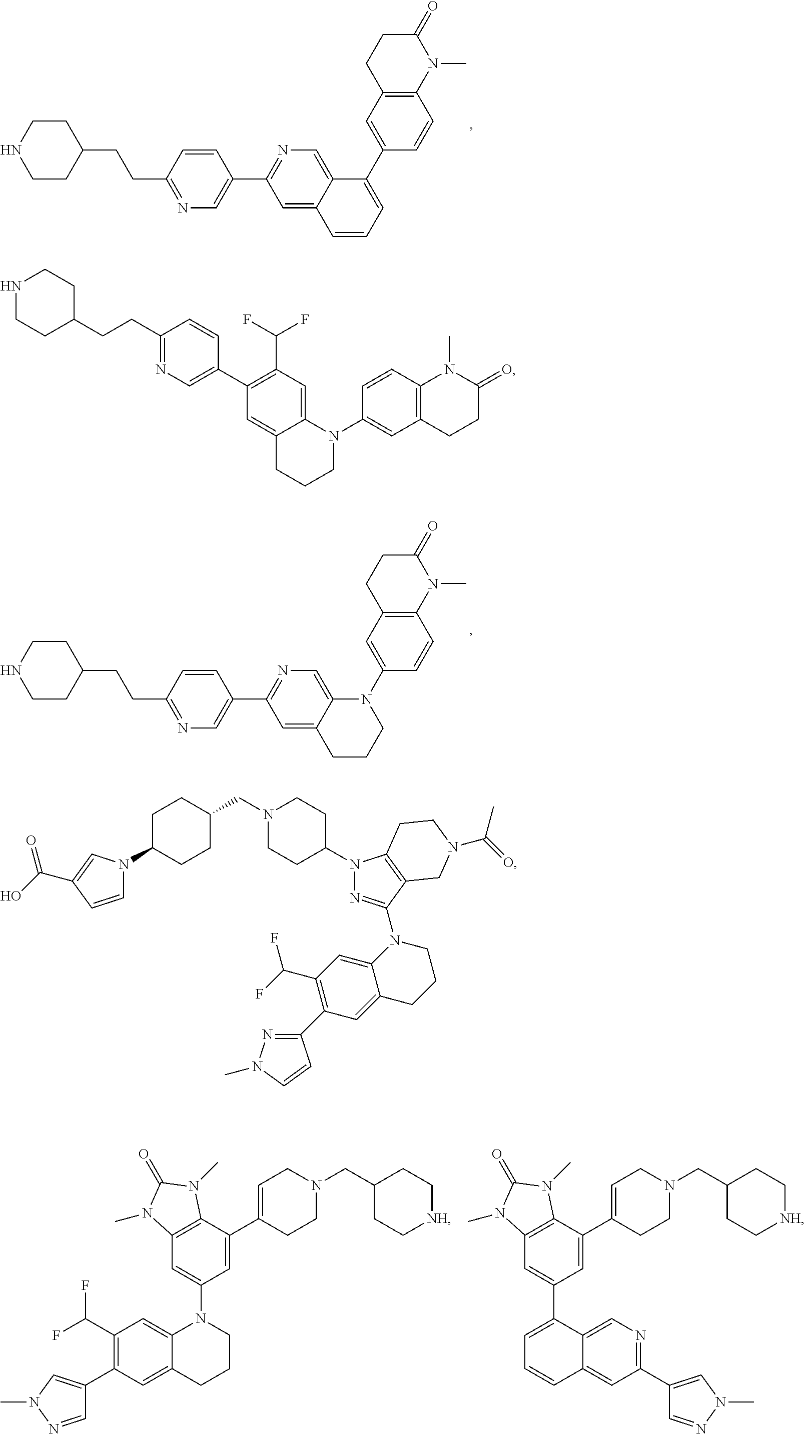

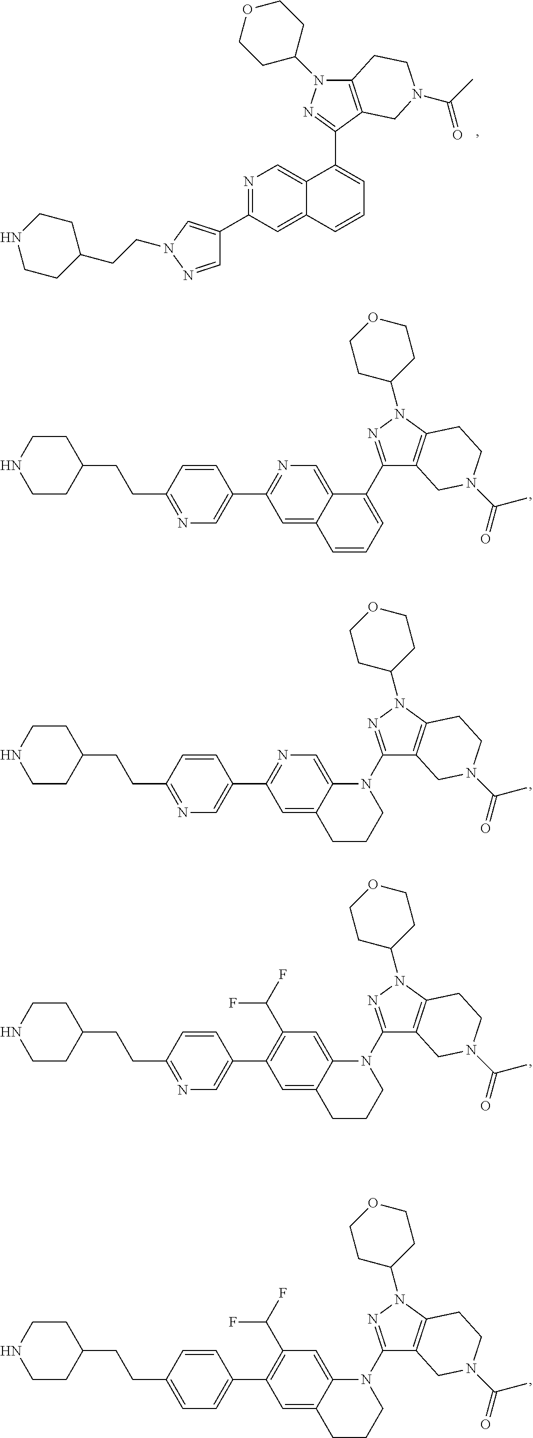

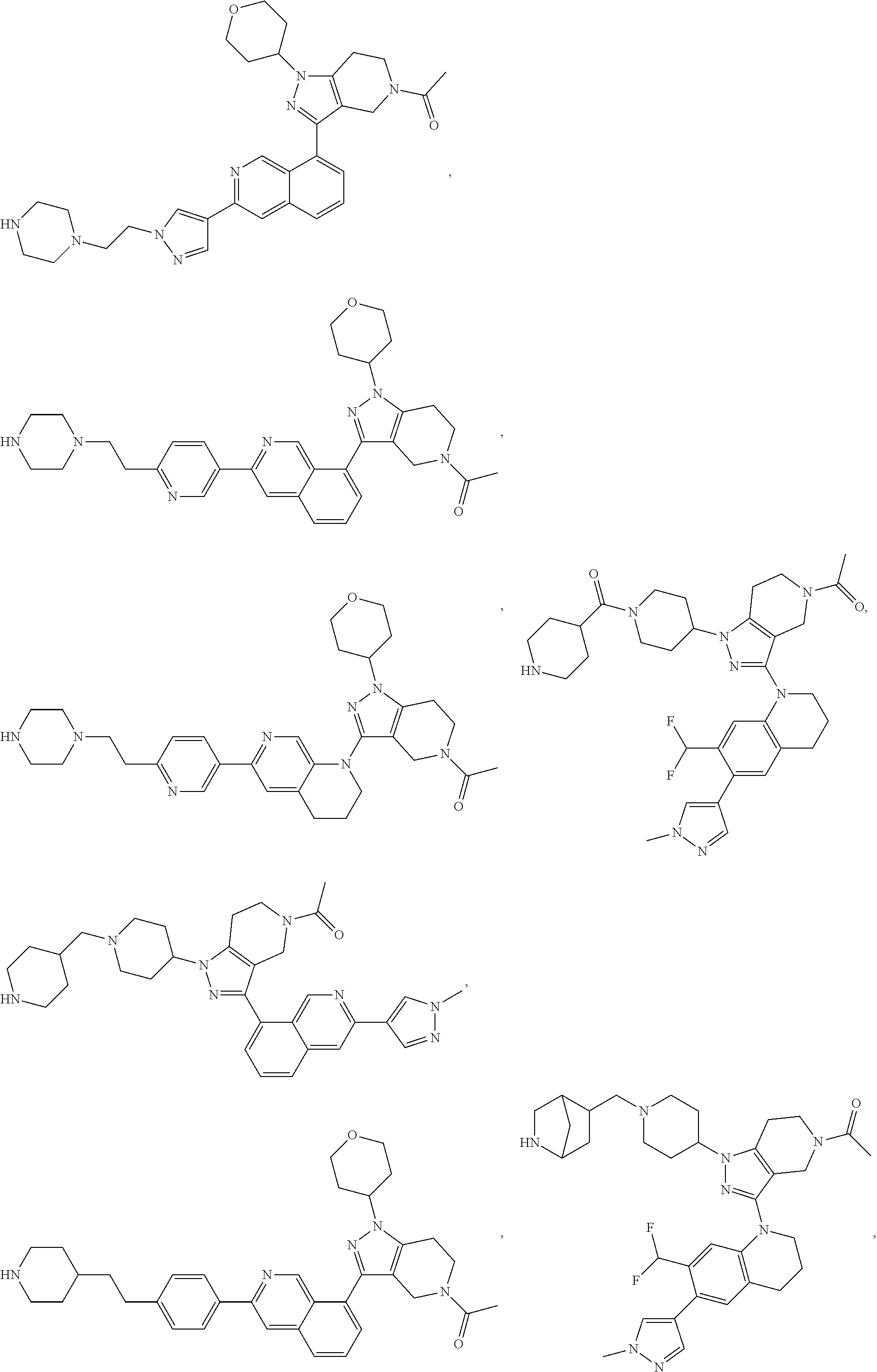

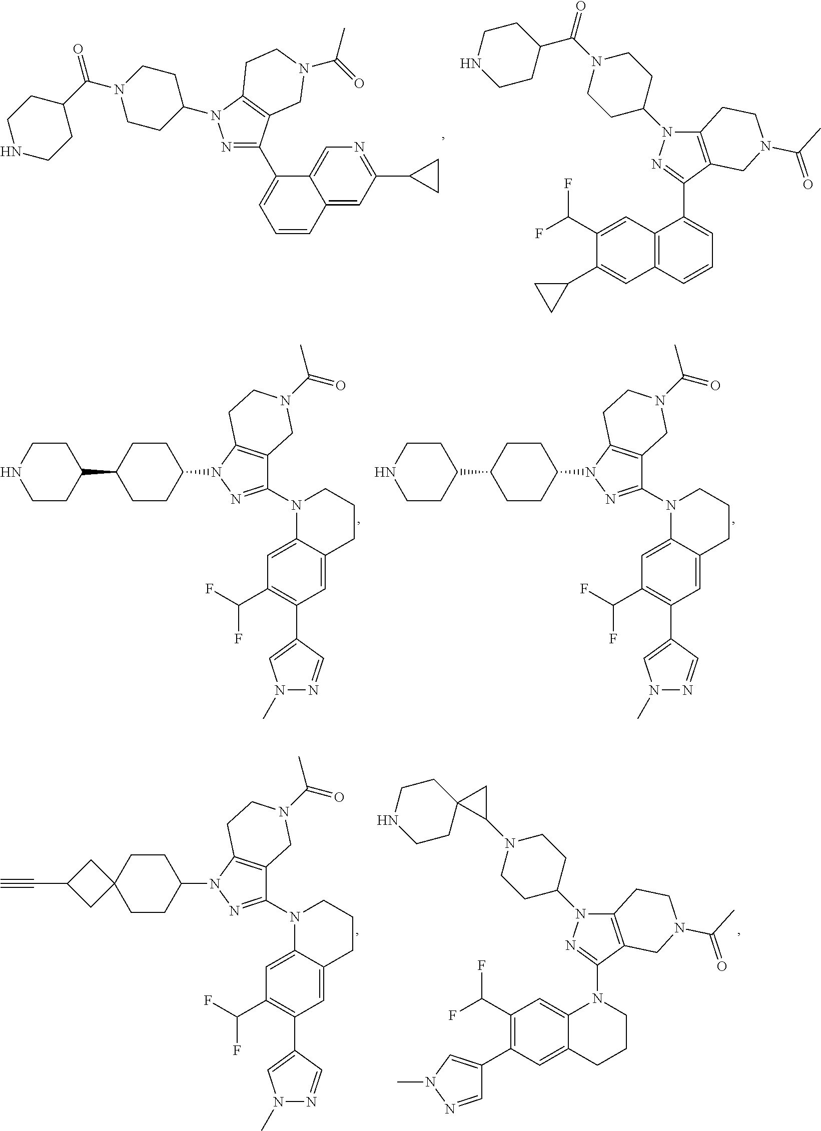 [SMILES: CC(=O)N1CCc2c(c(-c3cccc4cc(C5CC5)ncc34)nn2C2CCN(C(=O)C3CCNCC3)CC2)C1 ; CC(=O)N1CCc2c(c(-c3cccc4cc(C5CC5)c(C(F)F)cc34)nn2C2CCN(C(=O)C3CCNCC3)CC2)C1 ; CC(=O)N1CCc2c(c(N3CCCc4cc(-c5cnn(C)c5)c(C(F)F)cc43)nn2[C@H]2CC[C@H](C3CCNCC3)CC2)C1 ; CC(=O)N1CCc2c(c(N3CCCc4cc(-c5cnn(C)c5)c(C(F)F)cc43)nn2[C@H]2CC[C@@H](C3CCNCC3)CC2)C1 ; C#CC1CC2(CCC(n3nc(N4CCCc5cc(-c6cnn(C)c6)c(C(F)F)cc54)c4c3CCN(C(C)=O)C4)CC2)C1 ; CC(=O)N1CCc2c(c(N3CCCc4cc(-c5cnn(C)c5)c(C(F)F)cc43)nn2C2CCN(C3CC34CCNCC4)CC2)C1]

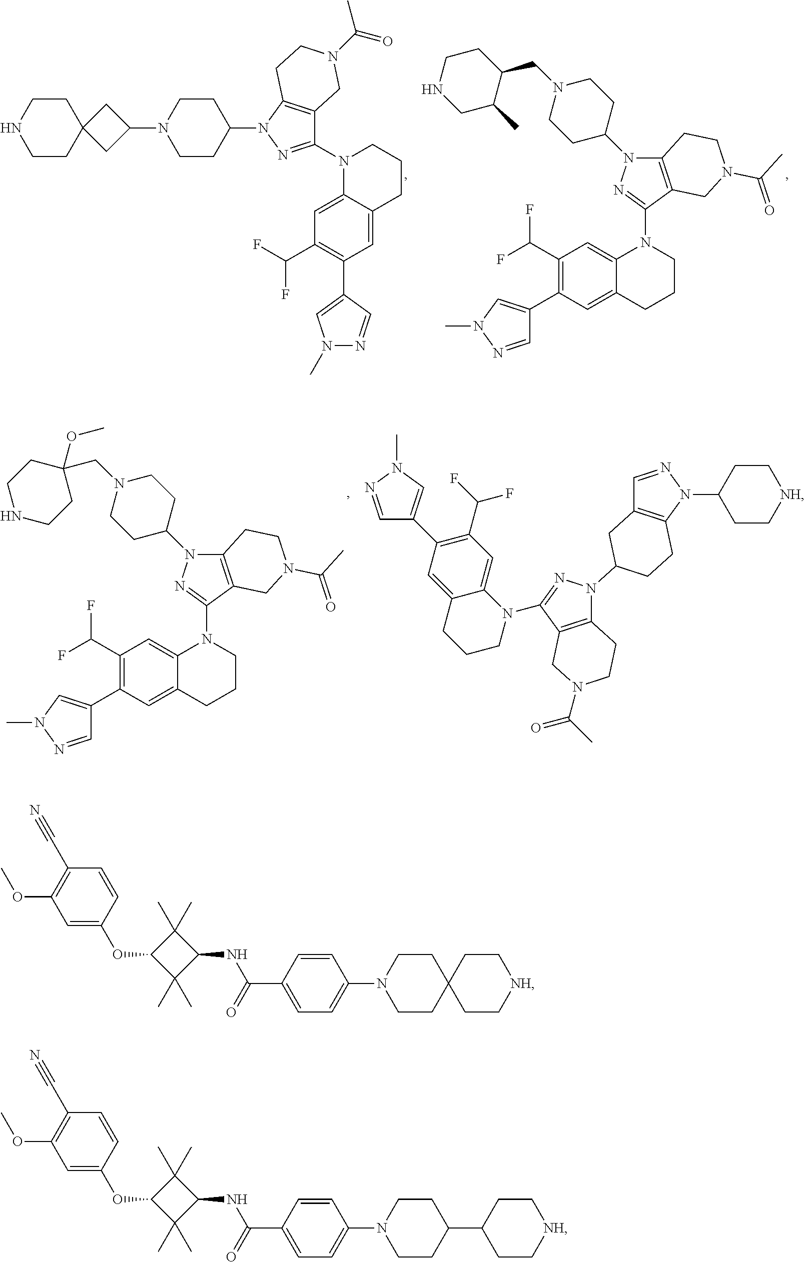 [SMILES: CC(=O)N1CCc2c(c(N3CCCc4cc(-c5cnn(C)c5)c(C(F)F)cc43)nn2C2CCN(C3CC4(CCNCC4)C3)CC2)C1 ; CC(=O)N1CCc2c(c(N3CCCc4cc(-c5cnn(C)c5)c(C(F)F)cc43)nn2C2CCN(C[C@@H]3CCNC[C@@H]3C)CC2)C1 ; COC1(CN2CCC(n3nc(N4CCCc5cc(-c6cnn(C)c6)c(C(F)F)cc54)c4c3CCN(C(C)=O)C4)CC2)CCNCC1 ; CC(=O)N1CCc2c(c(N3CCCc4cc(-c5cnn(C)c5)c(C(F)F)cc43)nn2C2CCc3c(cnn3C3CCNCC3)C2)C1 ; COc1cc(O[C@H]2C(C)(C)[C@H](NC(=O)c3ccc(N4CCC5(CCNCC5)CC4)cc3)C2(C)C)ccc1C#N ; COc1cc(O[C@H]2C(C)(C)[C@H](NC(=O)c3ccc(N4CCC(C5CCNCC5)CC4)cc3)C2(C)C)ccc1C#N]

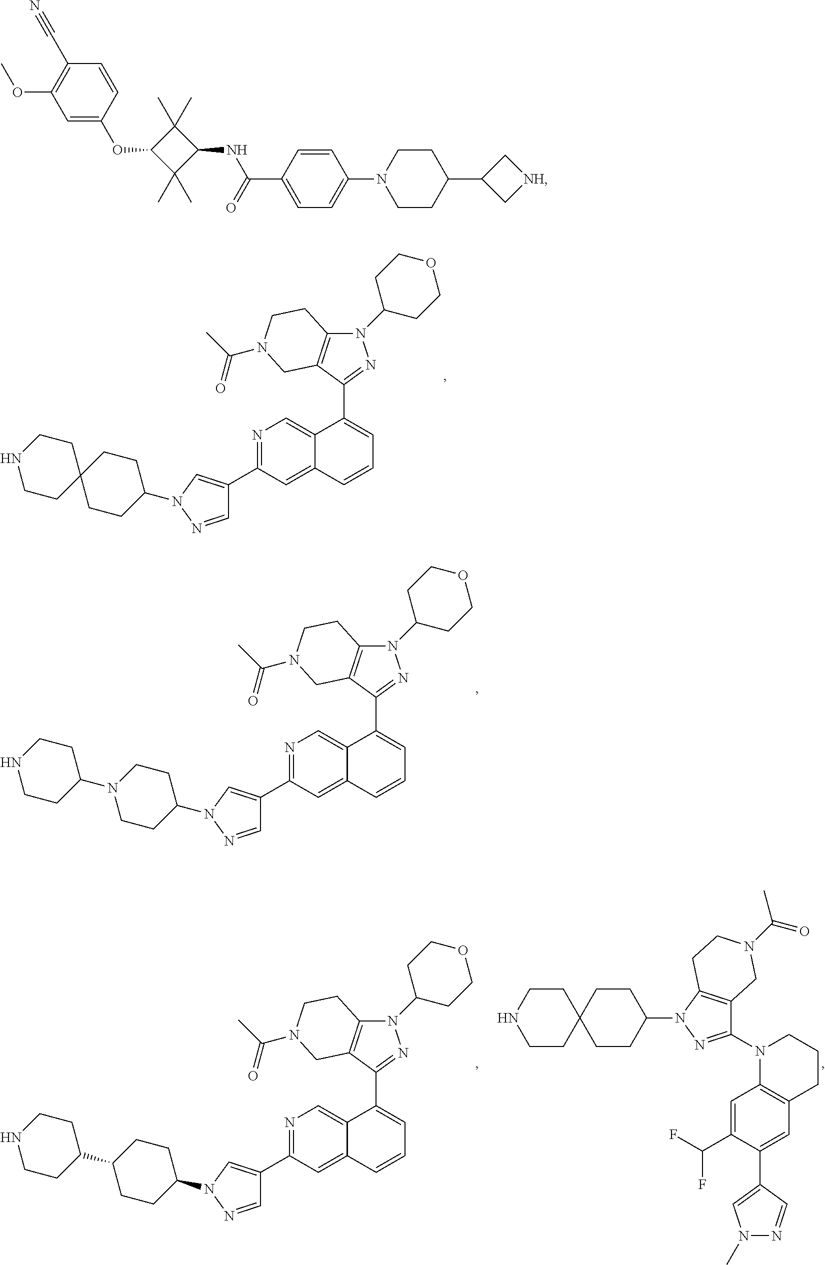

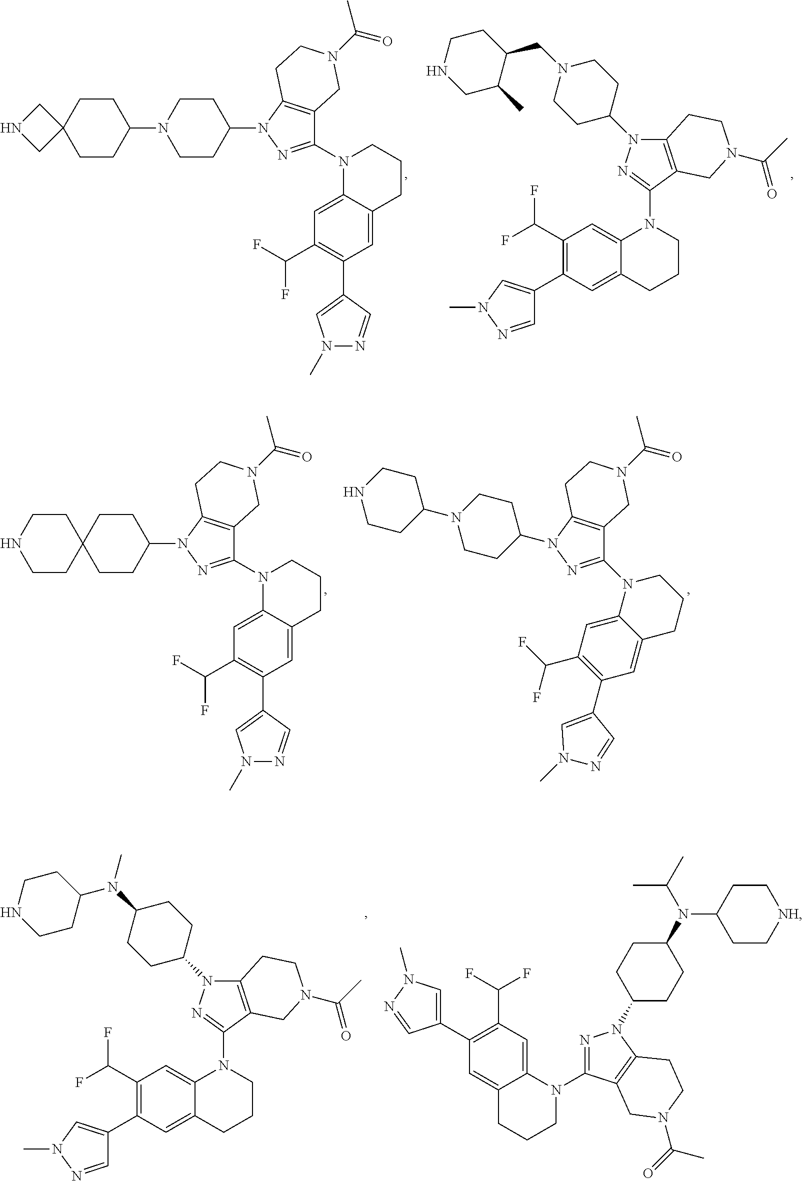

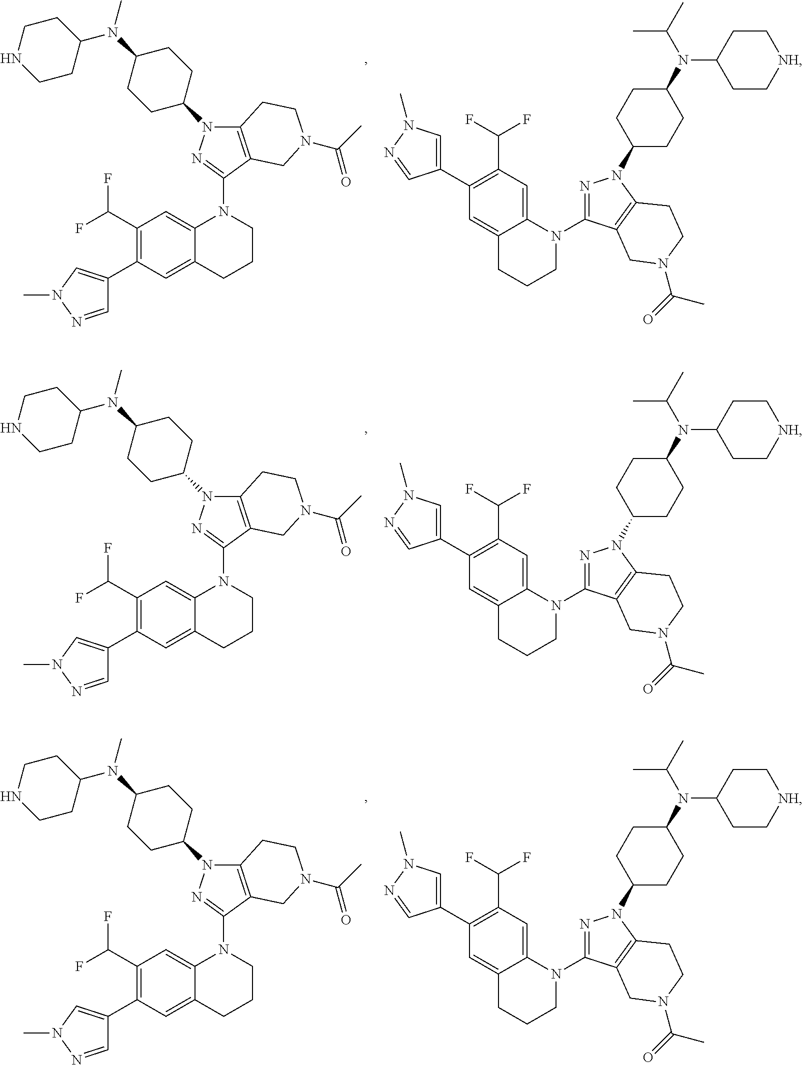

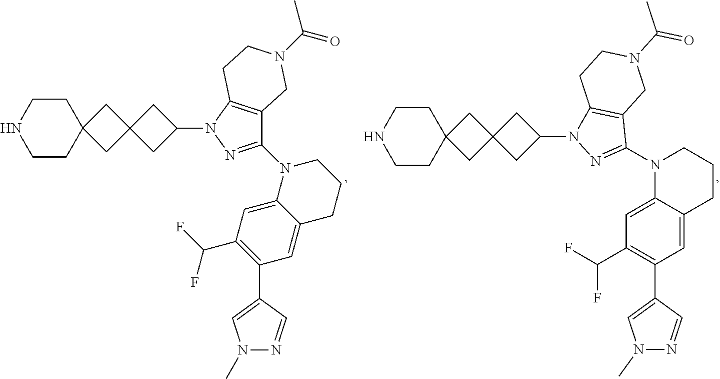 [SMILES: CC(=O)N1CCc2c(c(N3CCCc4cc(-c5cnn(C)c5)c(C(F)F)cc43)nn2C2CC3(C2)CC2(CCNCC2)C3)C1 ; CC(=O)N1CCc2c(c(N3CCCc4cc(-c5cnn(C)c5)c(C(F)F)cc43)nn2C2CC3(C2)CC2(CCNCC2)C3)C1]

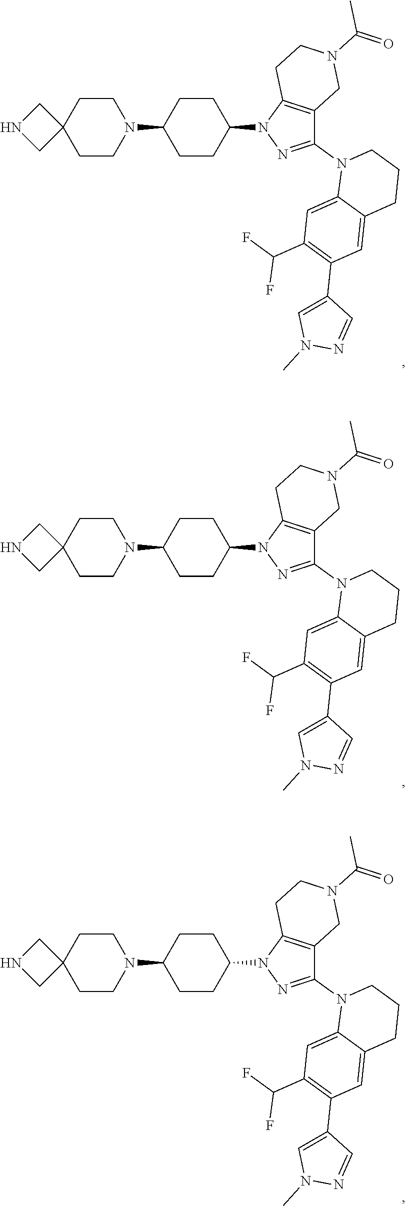 [SMILES: CC(=O)N1CCc2c(c(N3CCCc4cc(-c5cnn(C)c5)c(C(F)F)cc43)nn2[C@H]2CC[C@@H](N3CCC4(CC3)CNC4)CC2)C1 ; CC(=O)N1CCc2c(c(N3CCCc4cc(-c5cnn(C)c5)c(C(F)F)cc43)nn2[C@H]2CC[C@@H](N3CCC4(CC3)CNC4)CC2)C1 ; CC(=O)N1CCc2c(c(N3CCCc4cc(-c5cnn(C)c5)c(C(F)F)cc43)nn2[C@H]2CC[C@H](N3CCC4(CC3)CNC4)CC2)C1]

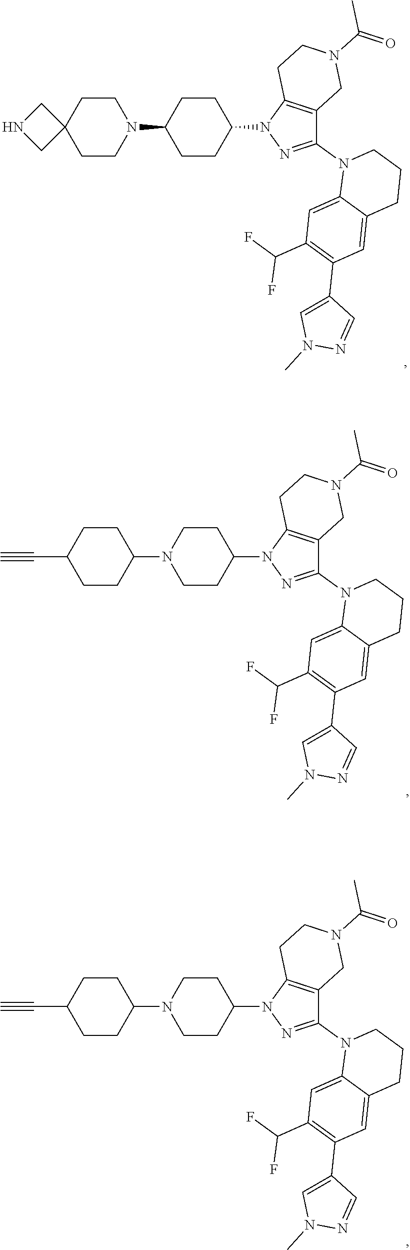 [SMILES: CC(=O)N1CCc2c(c(N3CCCc4cc(-c5cnn(C)c5)c(C(F)F)cc43)nn2[C@H]2CC[C@H](N3CCC4(CC3)CNC4)CC2)C1 ; C#CC1CCC(N2CCC(n3nc(N4CCCc5cc(-c6cnn(C)c6)c(C(F)F)cc54)c4c3CCN(C(C)=O)C4)CC2)CC1 ; C#CC1CCC(N2CCC(n3nc(N4CCCc5cc(-c6cnn(C)c6)c(C(F)F)cc54)c4c3CCN(C(C)=O)C4)CC2)CC1]

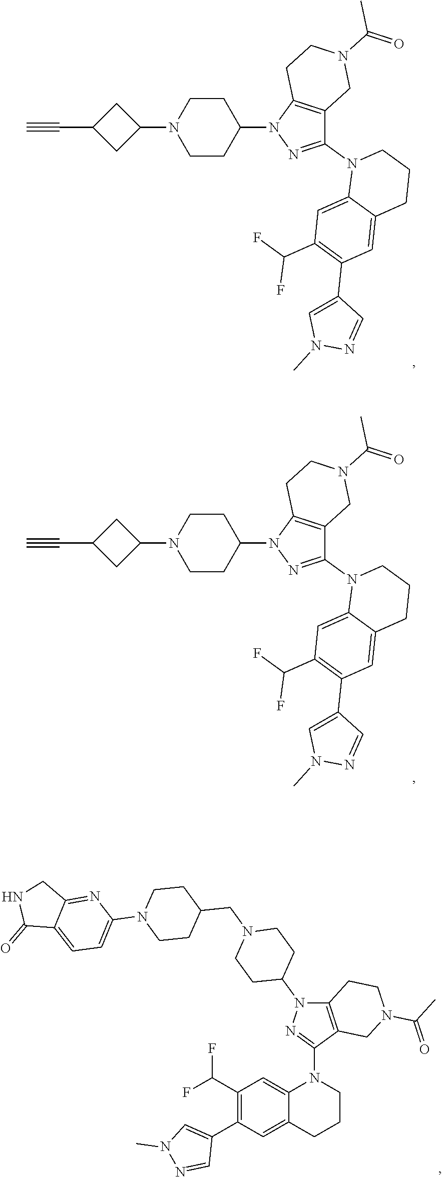 [SMILES: C#CC1CC(N2CCC(n3nc(N4CCCc5cc(-c6cnn(C)c6)c(C(F)F)cc54)c4c3CCN(C(C)=O)C4)CC2)C1 ; C#CC1CC(N2CCC(n3nc(N4CCCc5cc(-c6cnn(C)c6)c(C(F)F)cc54)c4c3CCN(C(C)=O)C4)CC2)C1 ; CC(=O)N1CCc2c(c(N3CCCc4cc(-c5cnn(C)c5)c(C(F)F)cc43)nn2C2CCN(CC3CCN(c4ccc5c(n4)CNC5=O)CC3)CC2)C1]

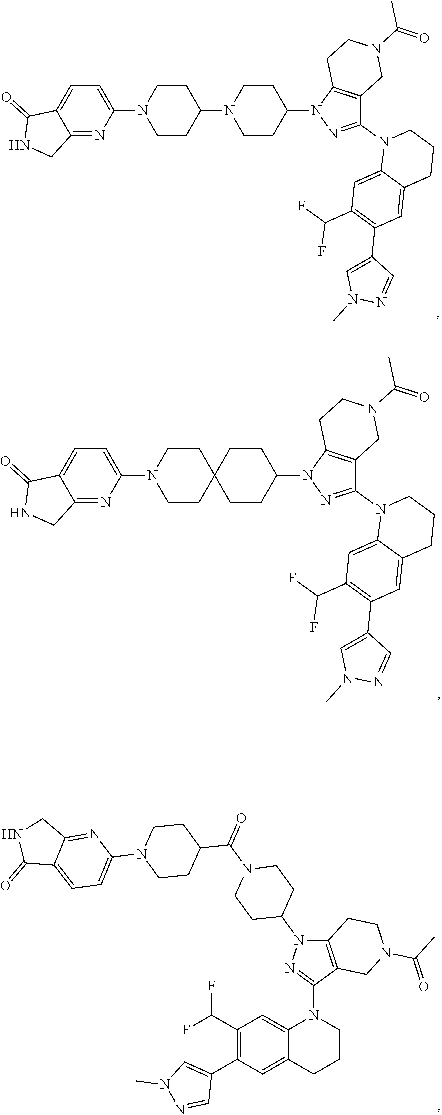 [SMILES: CC(=O)N1CCc2c(c(N3CCCc4cc(-c5cnn(C)c5)c(C(F)F)cc43)nn2C2CCN(C3CCN(c4ccc5c(n4)CNC5=O)CC3)CC2)C1 ; CC(=O)N1CCc2c(c(N3CCCc4cc(-c5cnn(C)c5)c(C(F)F)cc43)nn2C2CCC3(CC2)CCN(c2ccc4c(n2)CNC4=O)CC3)C1 ; CC(=O)N1CCc2c(c(N3CCCc4cc(-c5cnn(C)c5)c(C(F)F)cc43)nn2C2CCN(C(=O)C3CCN(c4ccc5c(n4)CNC5=O)CC3)CC2)C1]

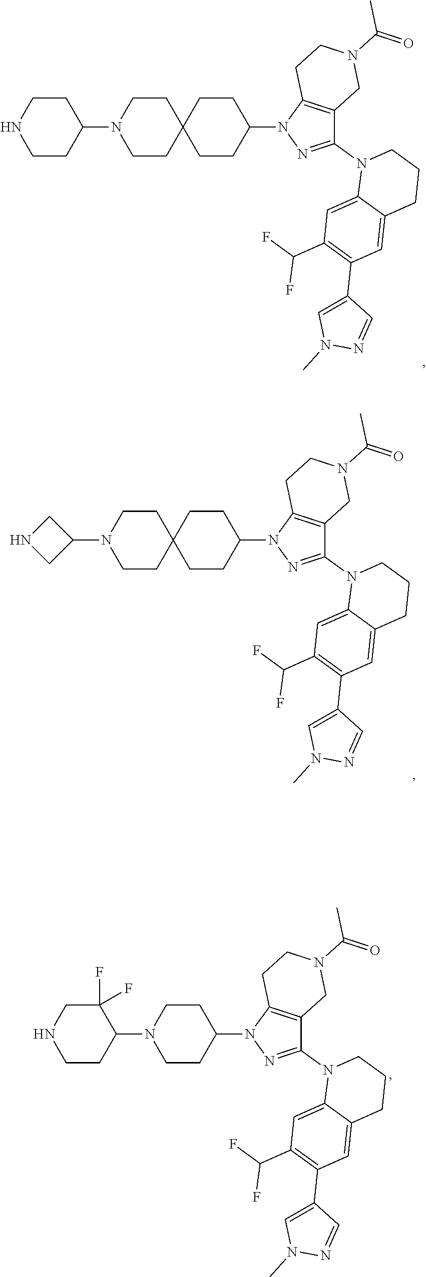 [SMILES: CC(=O)N1CCc2c(c(N3CCCc4cc(-c5cnn(C)c5)c(C(F)F)cc43)nn2C2CCC3(CC2)CCN(C2CCNCC2)CC3)C1 ; CC(=O)N1CCc2c(c(N3CCCc4cc(-c5cnn(C)c5)c(C(F)F)cc43)nn2C2CCC3(CC2)CCN(C2CNC2)CC3)C1 ; CC(=O)N1CCc2c(c(N3CCCc4cc(-c5cnn(C)c5)c(C(F)F)cc43)nn2C2CCN(C3CCNCC3(F)F)CC2)C1]

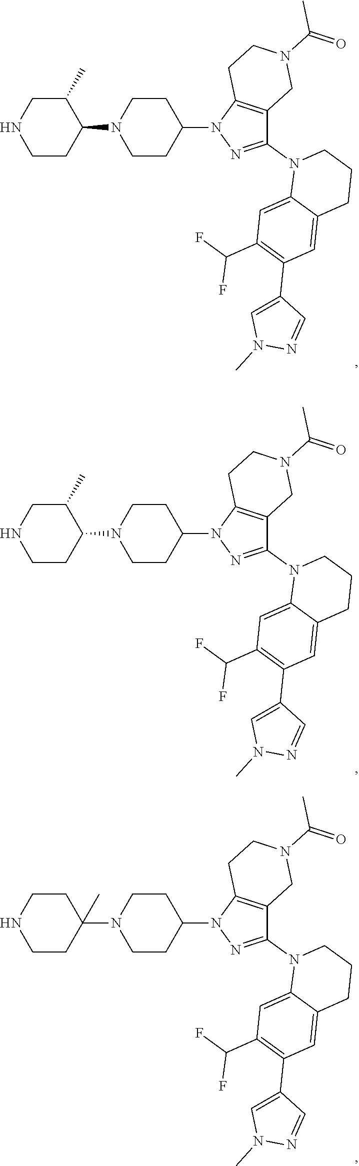

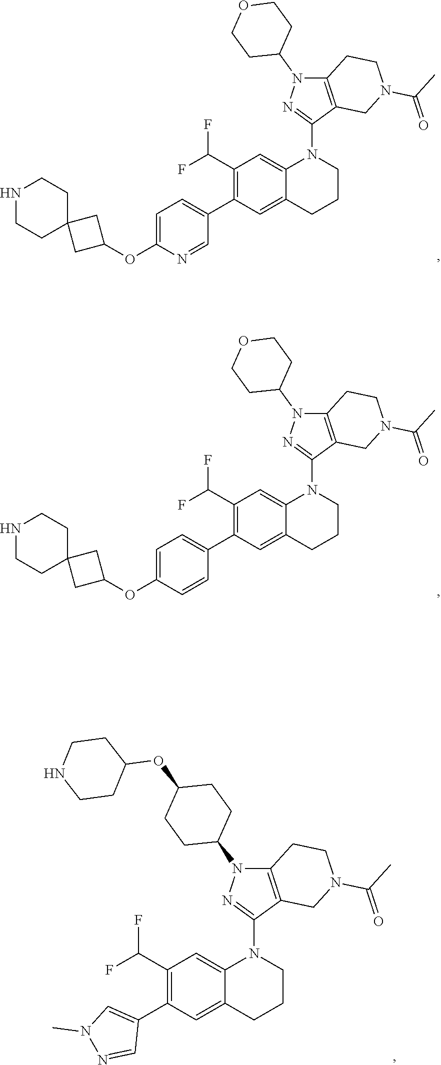

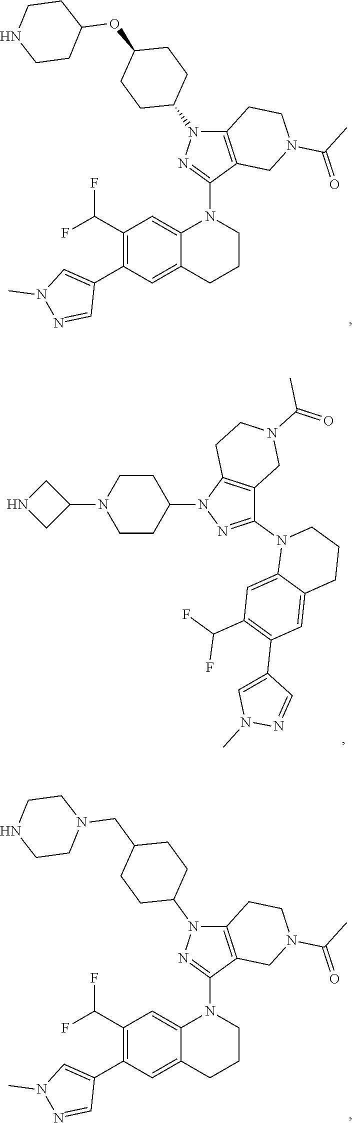

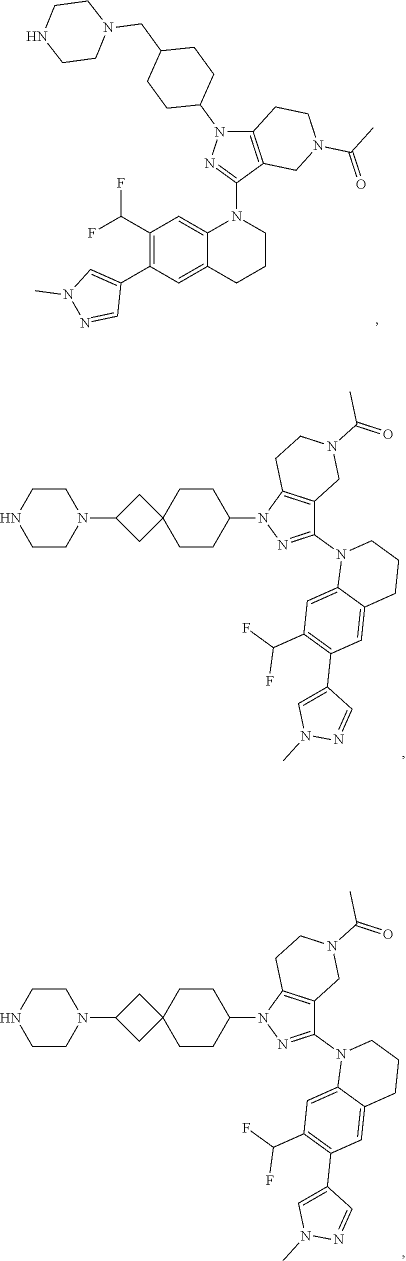 [SMILES: CC(=O)N1CCc2c(c(N3CCCc4cc(-c5cnn(C)c5)c(C(F)F)cc43)nn2C2CCC(CN3CCNCC3)CC2)C1 ; CC(=O)N1CCc2c(c(N3CCCc4cc(-c5cnn(C)c5)c(C(F)F)cc43)nn2C2CCC3(CC2)CC(N2CCNCC2)C3)C1 ; CC(=O)N1CCc2c(c(N3CCCc4cc(-c5cnn(C)c5)c(C(F)F)cc43)nn2C2CCC3(CC2)CC(N2CCNCC2)C3)C1]

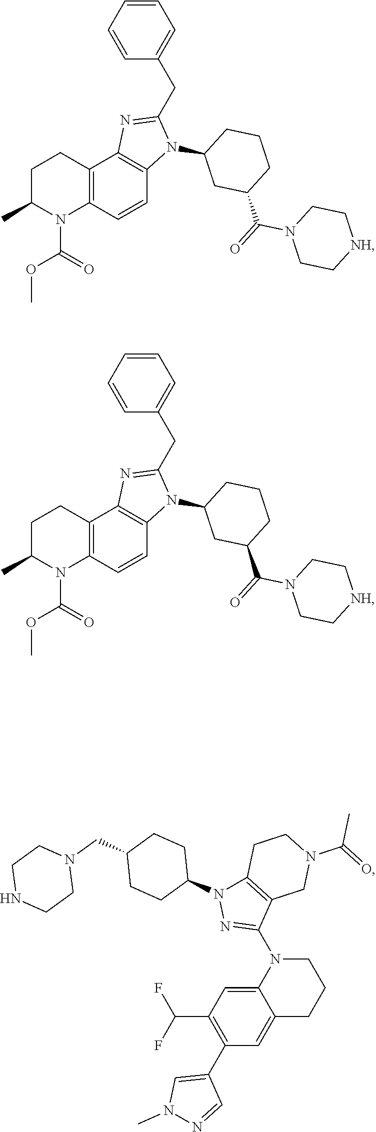 [SMILES: COC(=O)N1c2ccc3c(nc(Cc4ccccc4)n3[C@H]3CCC[C@H](C(=O)N4CCNCC4)C3)c2CC[C@@H]1C ; COC(=O)N1c2ccc3c(nc(Cc4ccccc4)n3[C@H]3CCC[C@@H](C(=O)N4CCNCC4)C3)c2CC[C@@H]1C ; CC(=O)N1CCc2c(c(N3CCCc4cc(-c5cnn(C)c5)c(C(F)F)cc43)nn2[C@H]2CC[C@H](CN3CCNCC3)CC2)C1]

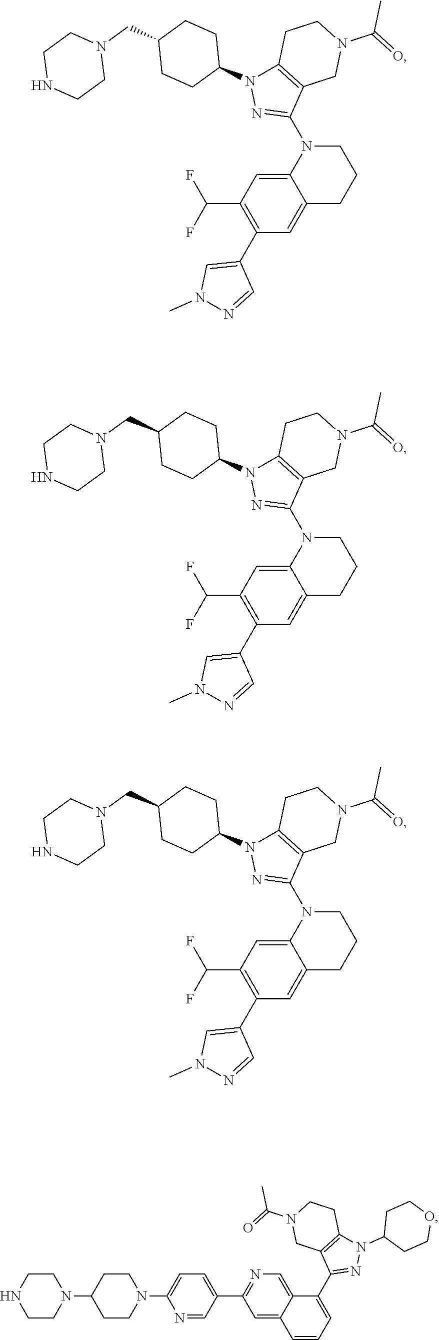 [SMILES: CC(=O)N1CCc2c(c(N3CCCc4cc(-c5cnn(C)c5)c(C(F)F)cc43)nn2[C@H]2CC[C@H](CN3CCNCC3)CC2)C1 ; CC(=O)N1CCc2c(c(N3CCCc4cc(-c5cnn(C)c5)c(C(F)F)cc43)nn2[C@H]2CC[C@@H](CN3CCNCC3)CC2)C1 ; CC(=O)N1CCc2c(c(N3CCCc4cc(-c5cnn(C)c5)c(C(F)F)cc43)nn2[C@H]2CC[C@@H](CN3CCNCC3)CC2)C1 ; CC(=O)N1CCc2c(c(-c3cccc4cc(-c5ccc(N6CCC(N7CCNCC7)CC6)nc5)ncc34)nn2C2CCOCC2)C1]

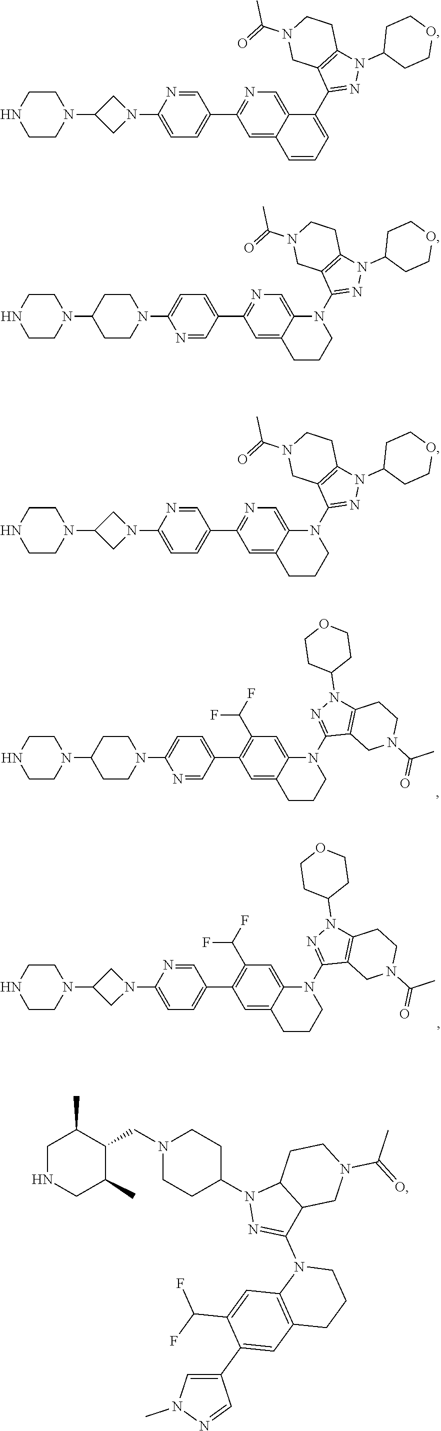 [SMILES: CC(=O)N1CCc2c(c(-c3cccc4cc(-c5ccc(N6CC(N7CCNCC7)C6)nc5)ncc34)nn2C2CCOCC2)C1 ; CC(=O)N1CCc2c(c(N3CCCc4cc(-c5ccc(N6CCC(N7CCNCC7)CC6)nc5)ncc43)nn2C2CCOCC2)C1 ; CC(=O)N1CCc2c(c(N3CCCc4cc(-c5ccc(N6CC(N7CCNCC7)C6)nc5)ncc43)nn2C2CCOCC2)C1 ; CC(=O)N1CCc2c(c(N3CCCc4cc(-c5ccc(N6CCC(N7CCNCC7)CC6)nc5)c(C(F)F)cc43)nn2C2CCOCC2)C1 ; CC(=O)N1CCc2c(c(N3CCCc4cc(-c5ccc(N6CC(N7CCNCC7)C6)nc5)c(C(F)F)cc43)nn2C2CCOCC2)C1 ; CC(=O)N1CCC2C(C1)C(N1CCCc3cc(-c4cnn(C)c4)c(C(F)F)cc31)=NN2C1CCN(C[C@H]2[C@H](C)CNC[C@@H]2C)CC1]

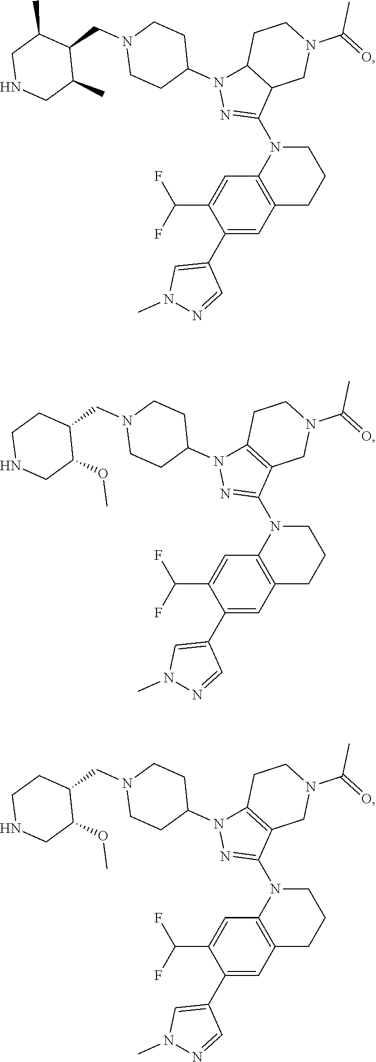 [SMILES: CC(=O)N1CCC2C(C1)C(N1CCCc3cc(-c4cnn(C)c4)c(C(F)F)cc31)=NN2C1CCN(C[C@@H]2[C@H](C)CNC[C@@H]2C)CC1 ; CO[C@@H]1CNCC[C@@H]1CN1CCC(n2nc(N3CCCc4cc(-c5cnn(C)c5)c(C(F)F)cc43)c3c2CCN(C(C)=O)C3)CC1 ; CO[C@@H]1CNCC[C@@H]1CN1CCC(n2nc(N3CCCc4cc(-c5cnn(C)c5)c(C(F)F)cc43)c3c2CCN(C(C)=O)C3)CC1]

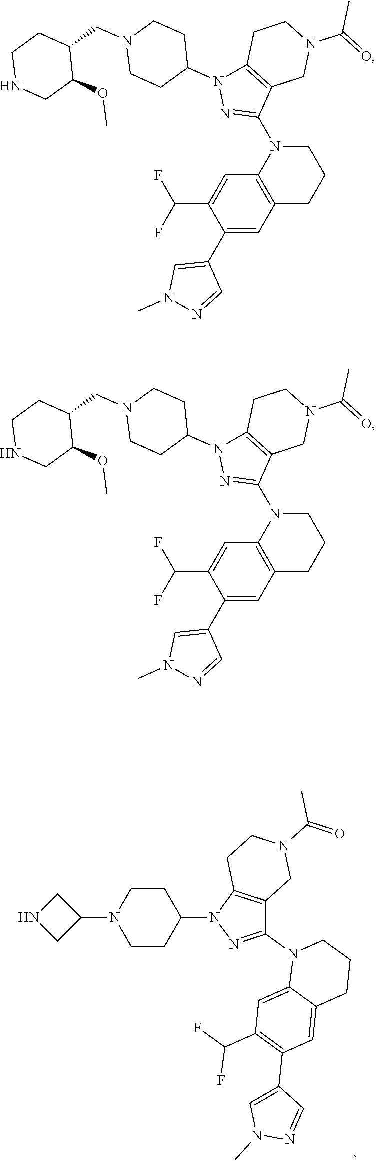 [SMILES: CO[C@H]1CNCC[C@@H]1CN1CCC(n2nc(N3CCCc4cc(-c5cnn(C)c5)c(C(F)F)cc43)c3c2CCN(C(C)=O)C3)CC1 ; CO[C@H]1CNCC[C@@H]1CN1CCC(n2nc(N3CCCc4cc(-c5cnn(C)c5)c(C(F)F)cc43)c3c2CCN(C(C)=O)C3)CC1 ; CC(=O)N1CCc2c(c(N3CCCc4cc(-c5cnn(C)c5)c(C(F)F)cc43)nn2C2CCN(C3CNC3)CC2)C1]

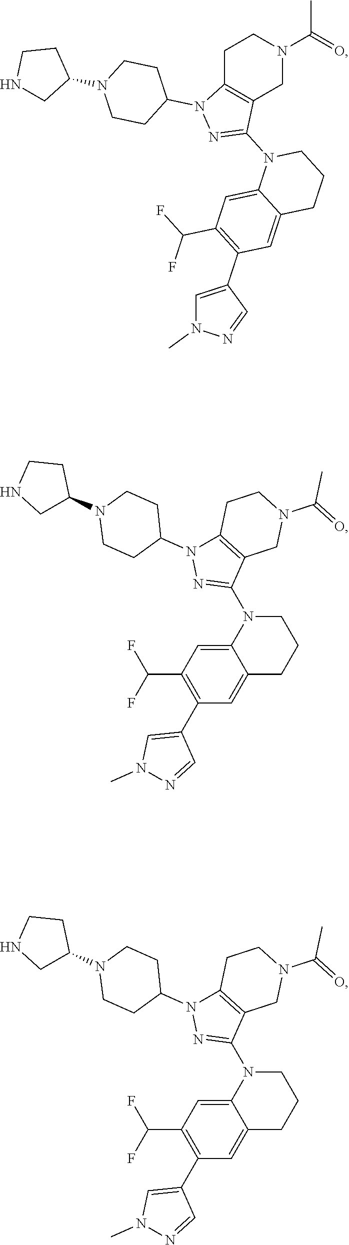 [SMILES: CC(=O)N1CCc2c(c(N3CCCc4cc(-c5cnn(C)c5)c(C(F)F)cc43)nn2C2CCN([C@H]3CCNC3)CC2)C1 ; CC(=O)N1CCc2c(c(N3CCCc4cc(-c5cnn(C)c5)c(C(F)F)cc43)nn2C2CCN([C@@H]3CCNC3)CC2)C1 ; CC(=O)N1CCc2c(c(N3CCCc4cc(-c5cnn(C)c5)c(C(F)F)cc43)nn2C2CCN([C@H]3CCNC3)CC2)C1]

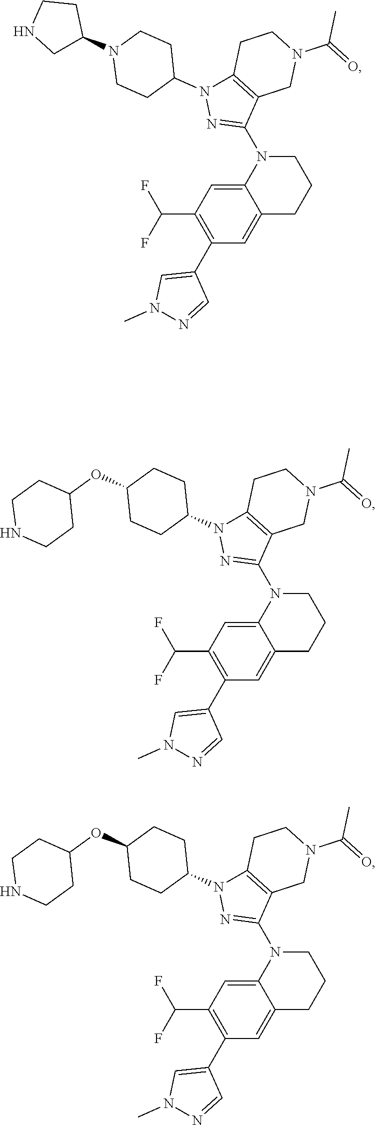 [SMILES: CC(=O)N1CCc2c(c(N3CCCc4cc(-c5cnn(C)c5)c(C(F)F)cc43)nn2C2CCN([C@@H]3CCNC3)CC2)C1 ; CC(=O)N1CCc2c(c(N3CCCc4cc(-c5cnn(C)c5)c(C(F)F)cc43)nn2[C@H]2CC[C@@H](OC3CCNCC3)CC2)C1 ; CC(=O)N1CCc2c(c(N3CCCc4cc(-c5cnn(C)c5)c(C(F)F)cc43)nn2[C@H]2CC[C@H](OC3CCNCC3)CC2)C1]

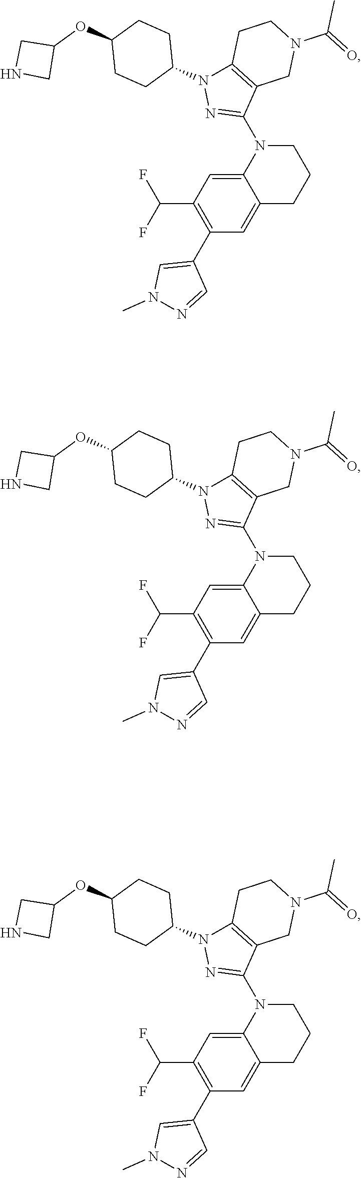 [SMILES: CC(=O)N1CCc2c(c(N3CCCc4cc(-c5cnn(C)c5)c(C(F)F)cc43)nn2[C@H]2CC[C@H](OC3CNC3)CC2)C1 ; CC(=O)N1CCc2c(c(N3CCCc4cc(-c5cnn(C)c5)c(C(F)F)cc43)nn2[C@H]2CC[C@@H](OC3CNC3)CC2)C1 ; CC(=O)N1CCc2c(c(N3CCCc4cc(-c5cnn(C)c5)c(C(F)F)cc43)nn2[C@H]2CC[C@H](OC3CNC3)CC2)C1]

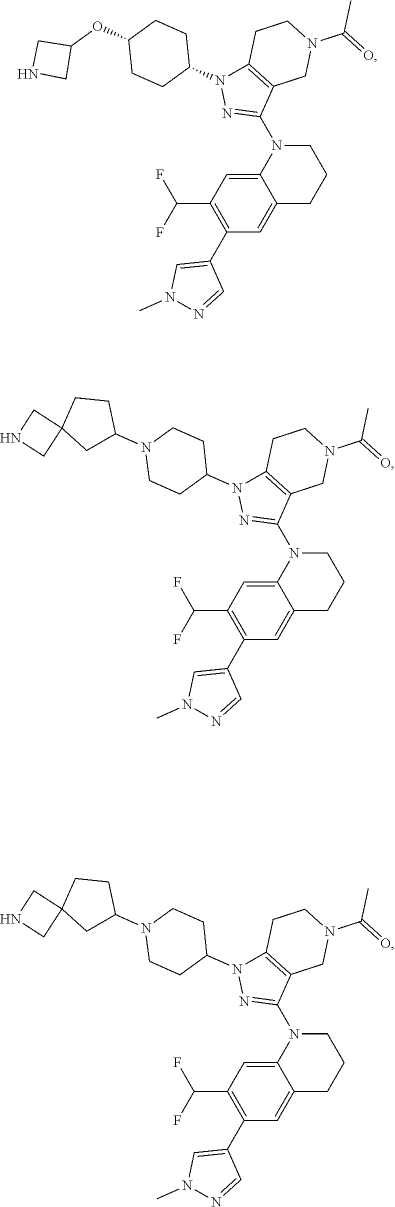 [SMILES: CC(=O)N1CCc2c(c(N3CCCc4cc(-c5cnn(C)c5)c(C(F)F)cc43)nn2[C@H]2CC[C@@H](OC3CNC3)CC2)C1 ; CC(=O)N1CCc2c(c(N3CCCc4cc(-c5cnn(C)c5)c(C(F)F)cc43)nn2C2CCN(C3CCC4(CNC4)C3)CC2)C1 ; CC(=O)N1CCc2c(c(N3CCCc4cc(-c5cnn(C)c5)c(C(F)F)cc43)nn2C2CCN(C3CCC4(CNC4)C3)CC2)C1]

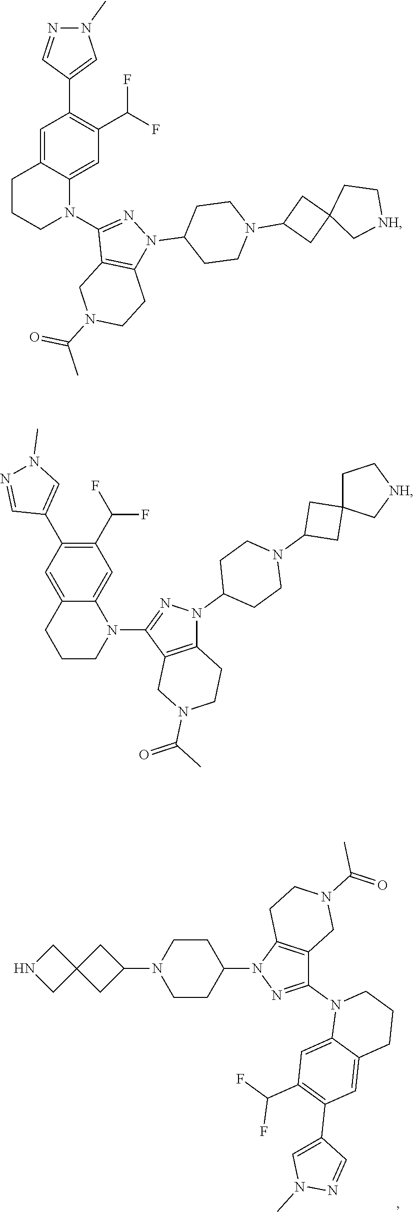 [SMILES: CC(=O)N1CCc2c(c(N3CCCc4cc(-c5cnn(C)c5)c(C(F)F)cc43)nn2C2CCN(C3CC4(CCNC4)C3)CC2)C1 ; CC(=O)N1CCc2c(c(N3CCCc4cc(-c5cnn(C)c5)c(C(F)F)cc43)nn2C2CCN(C3CC4(CCNC4)C3)CC2)C1 ; CC(=O)N1CCc2c(c(N3CCCc4cc(-c5cnn(C)c5)c(C(F)F)cc43)nn2C2CCN(C3CC4(CNC4)C3)CC2)C1]

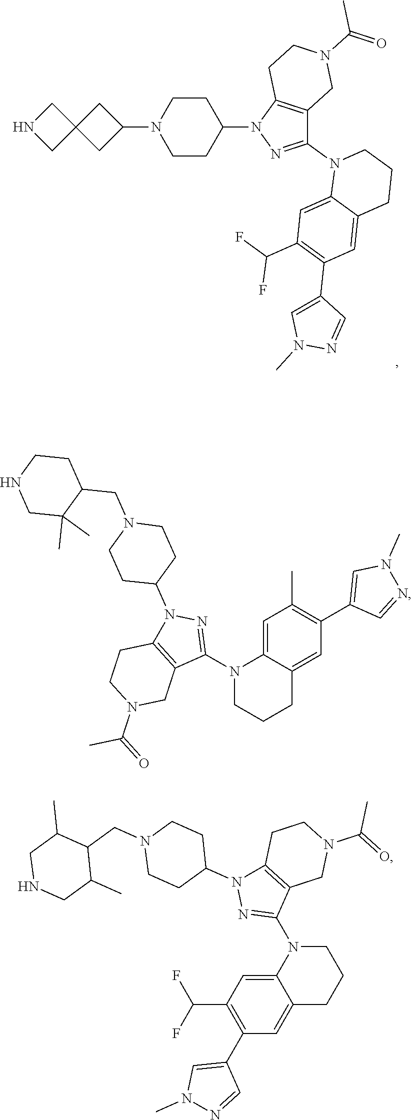 [SMILES: CC(=O)N1CCc2c(c(N3CCCc4cc(-c5cnn(C)c5)c(C(F)F)cc43)nn2C2CCN(C3CC4(CNC4)C3)CC2)C1 ; CC(=O)N1CCc2c(c(N3CCCc4cc(-c5cnn(C)c5)c(C)cc43)nn2C2CCN(CC3CCNCC3(C)C)CC2)C1 ; CC(=O)N1CCc2c(c(N3CCCc4cc(-c5cnn(C)c5)c(C(F)F)cc43)nn2C2CCN(CC3C(C)CNCC3C)CC2)C1]

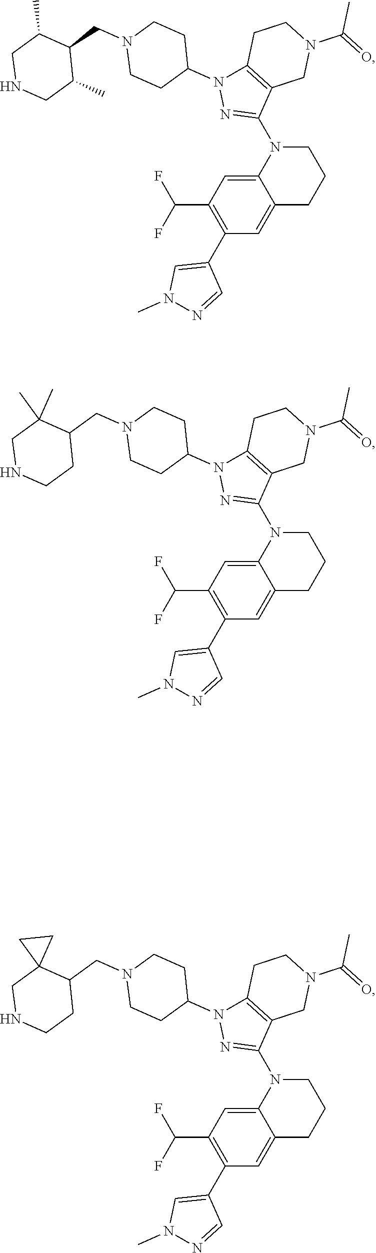 [SMILES: CC(=O)N1CCc2c(c(N3CCCc4cc(-c5cnn(C)c5)c(C(F)F)cc43)nn2C2CCN(C[C@H]3[C@H](C)CNC[C@@H]3C)CC2)C1 ; CC(=O)N1CCc2c(c(N3CCCc4cc(-c5cnn(C)c5)c(C(F)F)cc43)nn2C2CCN(CC3CCNCC3(C)C)CC2)C1 ; CC(=O)N1CCc2c(c(N3CCCc4cc(-c5cnn(C)c5)c(C(F)F)cc43)nn2C2CCN(CC3CCNCC34CC4)CC2)C1]

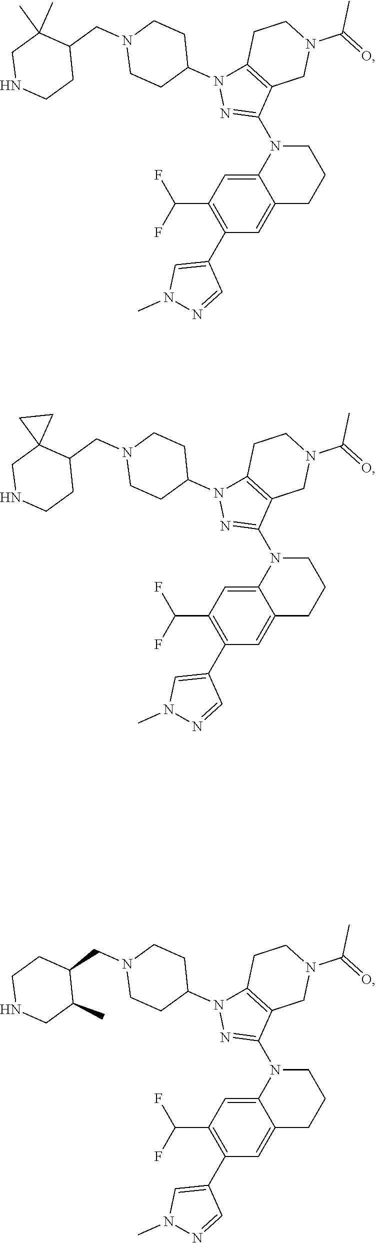 [SMILES: CC(=O)N1CCc2c(c(N3CCCc4cc(-c5cnn(C)c5)c(C(F)F)cc43)nn2C2CCN(CC3CCNCC3(C)C)CC2)C1 ; CC(=O)N1CCc2c(c(N3CCCc4cc(-c5cnn(C)c5)c(C(F)F)cc43)nn2C2CCN(CC3CCNCC34CC4)CC2)C1 ; CC(=O)N1CCc2c(c(N3CCCc4cc(-c5cnn(C)c5)c(C(F)F)cc43)nn2C2CCN(C[C@@H]3CCNC[C@@H]3C)CC2)C1]

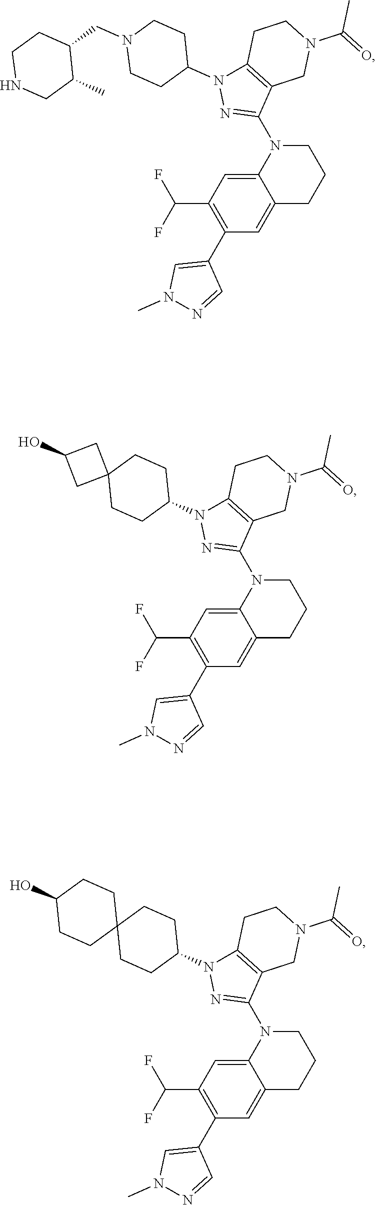 [SMILES: CC(=O)N1CCc2c(c(N3CCCc4cc(-c5cnn(C)c5)c(C(F)F)cc43)nn2C2CCN(C[C@H]3CCNC[C@H]3C)CC2)C1 ; CC(=O)N1CCc2c(c(N3CCCc4cc(-c5cnn(C)c5)c(C(F)F)cc43)nn2[C@H]2CCC3(CC2)C[C@@H](O)C3)C1 ; CC(=O)N1CCc2c(c(N3CCCc4cc(-c5cnn(C)c5)c(C(F)F)cc43)nn2[C@H]2CCC3(CC[C@@H](O)CC3)CC2)C1]

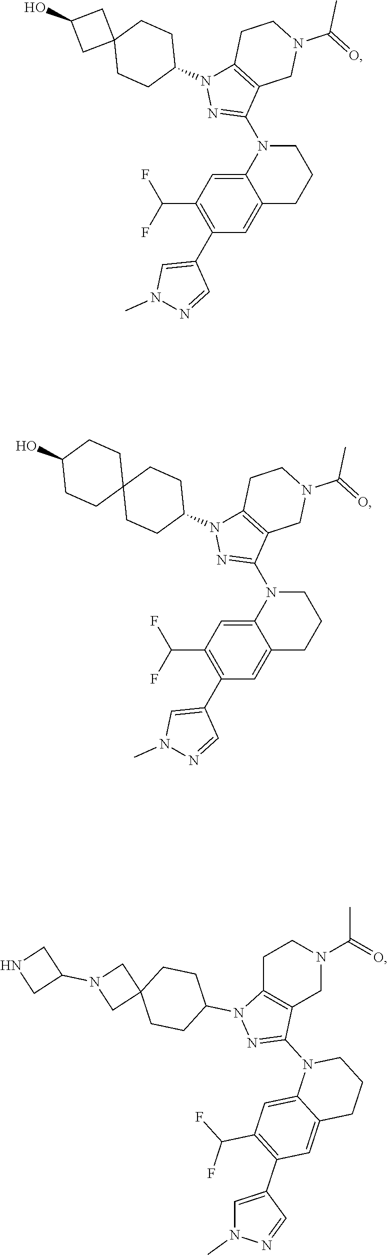 [SMILES: CC(=O)N1CCc2c(c(N3CCCc4cc(-c5cnn(C)c5)c(C(F)F)cc43)nn2[C@H]2CCC3(CC2)C[C@@H](O)C3)C1 ; CC(=O)N1CCc2c(c(N3CCCc4cc(-c5cnn(C)c5)c(C(F)F)cc43)nn2[C@H]2CCC3(CC[C@@H](O)CC3)CC2)C1 ; CC(=O)N1CCc2c(c(N3CCCc4cc(-c5cnn(C)c5)c(C(F)F)cc43)nn2C2CCC3(CC2)CN(C2CNC2)C3)C1]

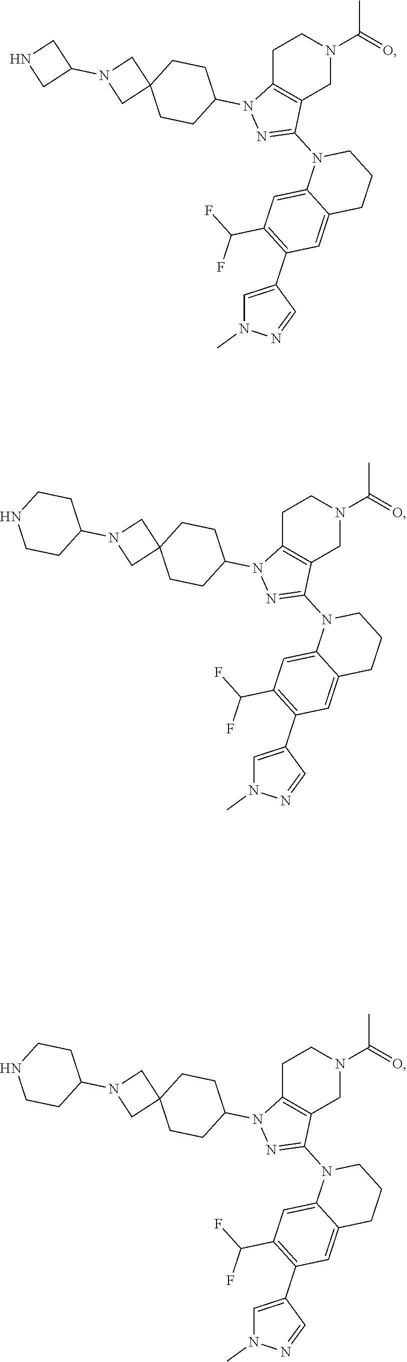 [SMILES: CC(=O)N1CCc2c(c(N3CCCc4cc(-c5cnn(C)c5)c(C(F)F)cc43)nn2C2CCC3(CC2)CN(C2CNC2)C3)C1 ; CC(=O)N1CCc2c(c(N3CCCc4cc(-c5cnn(C)c5)c(C(F)F)cc43)nn2C2CCC3(CC2)CN(C2CCNCC2)C3)C1 ; CC(=O)N1CCc2c(c(N3CCCc4cc(-c5cnn(C)c5)c(C(F)F)cc43)nn2C2CCC3(CC2)CN(C2CCNCC2)C3)C1]

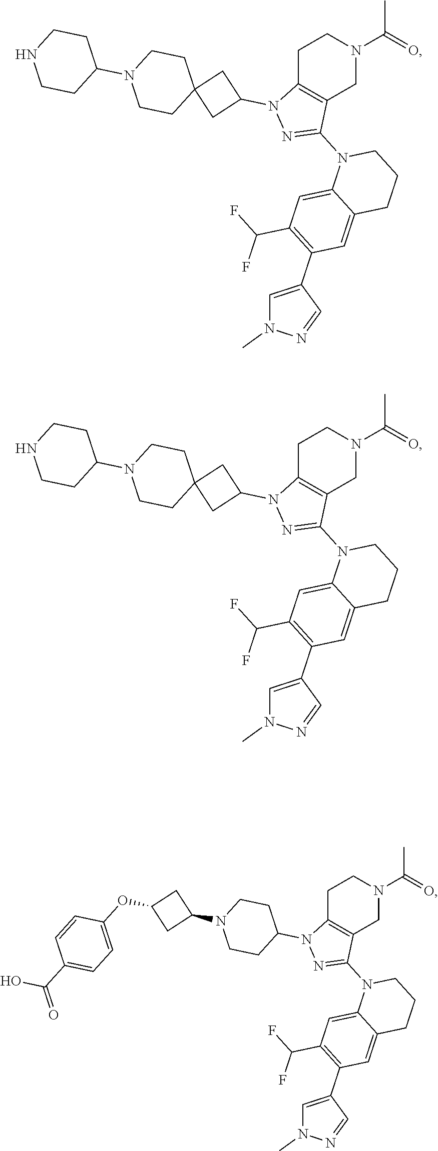 [SMILES: CC(=O)N1CCc2c(c(N3CCCc4cc(-c5cnn(C)c5)c(C(F)F)cc43)nn2C2CC3(CCN(C4CCNCC4)CC3)C2)C1 ; CC(=O)N1CCc2c(c(N3CCCc4cc(-c5cnn(C)c5)c(C(F)F)cc43)nn2C2CC3(CCN(C4CCNCC4)CC3)C2)C1 ; CC(=O)N1CCc2c(c(N3CCCc4cc(-c5cnn(C)c5)c(C(F)F)cc43)nn2C2CCN([C@H]3C[C@H](Oc4ccc(C(=O)O)cc4)C3)CC2)C1]

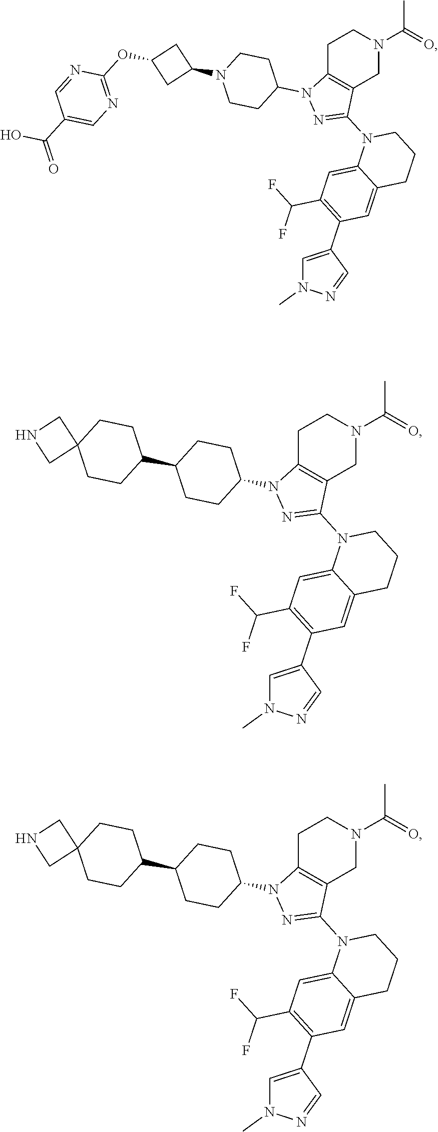 [SMILES: CC(=O)N1CCc2c(c(N3CCCc4cc(-c5cnn(C)c5)c(C(F)F)cc43)nn2C2CCN([C@H]3C[C@H](Oc4ncc(C(=O)O)cn4)C3)CC2)C1 ; CC(=O)N1CCc2c(c(N3CCCc4cc(-c5cnn(C)c5)c(C(F)F)cc43)nn2[C@H]2CC[C@H](C3CCC4(CC3)CNC4)CC2)C1 ; CC(=O)N1CCc2c(c(N3CCCc4cc(-c5cnn(C)c5)c(C(F)F)cc43)nn2[C@H]2CC[C@H](C3CCC4(CC3)CNC4)CC2)C1]

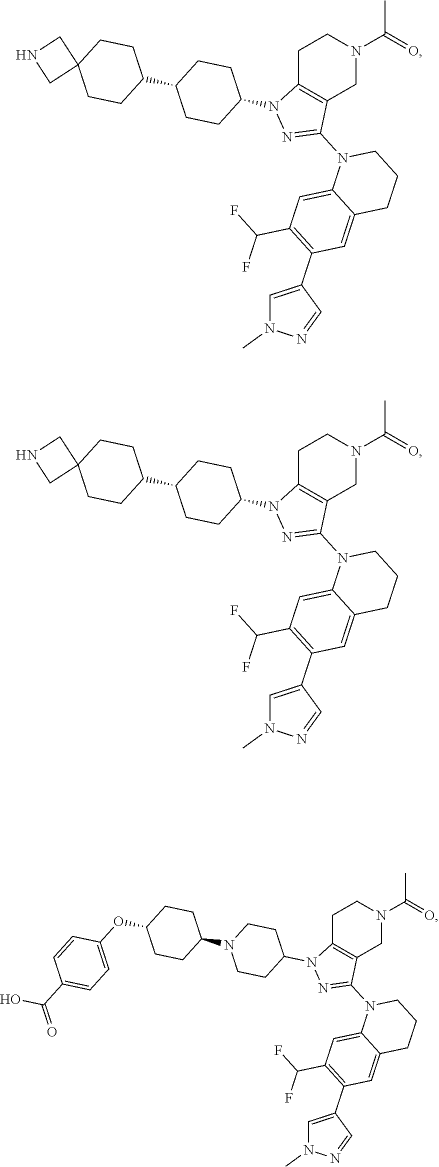 [SMILES: CC(=O)N1CCc2c(c(N3CCCc4cc(-c5cnn(C)c5)c(C(F)F)cc43)nn2[C@H]2CC[C@@H](C3CCC4(CC3)CNC4)CC2)C1 ; CC(=O)N1CCc2c(c(N3CCCc4cc(-c5cnn(C)c5)c(C(F)F)cc43)nn2[C@H]2CC[C@@H](C3CCC4(CC3)CNC4)CC2)C1 ; CC(=O)N1CCc2c(c(N3CCCc4cc(-c5cnn(C)c5)c(C(F)F)cc43)nn2C2CCN([C@H]3CC[C@H](Oc4ccc(C(=O)O)cc4)CC3)CC2)C1]

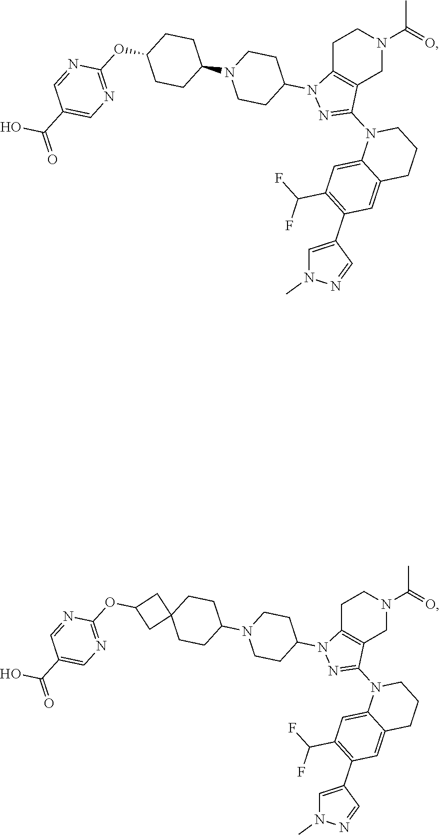 [SMILES: CC(=O)N1CCc2c(c(N3CCCc4cc(-c5cnn(C)c5)c(C(F)F)cc43)nn2C2CCN([C@H]3CC[C@H](Oc4ncc(C(=O)O)cn4)CC3)CC2)C1 ; CC(=O)N1CCc2c(c(N3CCCc4cc(-c5cnn(C)c5)c(C(F)F)cc43)nn2C2CCN(C3CCC4(CC3)CC(Oc3ncc(C(=O)O)cn3)C4)CC2)C1]

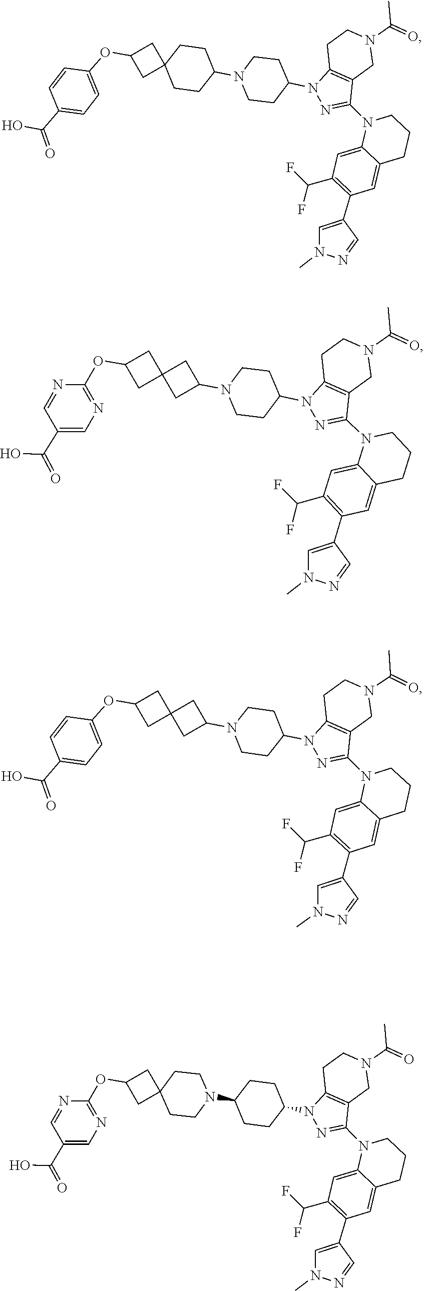 [SMILES: CC(=O)N1CCc2c(c(N3CCCc4cc(-c5cnn(C)c5)c(C(F)F)cc43)nn2C2CCN(C3CCC4(CC3)CC(Oc3ccc(C(=O)O)cc3)C4)CC2)C1 ; CC(=O)N1CCc2c(c(N3CCCc4cc(-c5cnn(C)c5)c(C(F)F)cc43)nn2C2CCN(C3CC4(CC(Oc5ncc(C(=O)O)cn5)C4)C3)CC2)C1 ; CC(=O)N1CCc2c(c(N3CCCc4cc(-c5cnn(C)c5)c(C(F)F)cc43)nn2C2CCN(C3CC4(CC(Oc5ccc(C(=O)O)cc5)C4)C3)CC2)C1 ; CC(=O)N1CCc2c(c(N3CCCc4cc(-c5cnn(C)c5)c(C(F)F)cc43)nn2[C@H]2CC[C@H](N3CCC4(CC3)CC(Oc3ncc(C(=O)O)cn3)C4)CC2)C1]

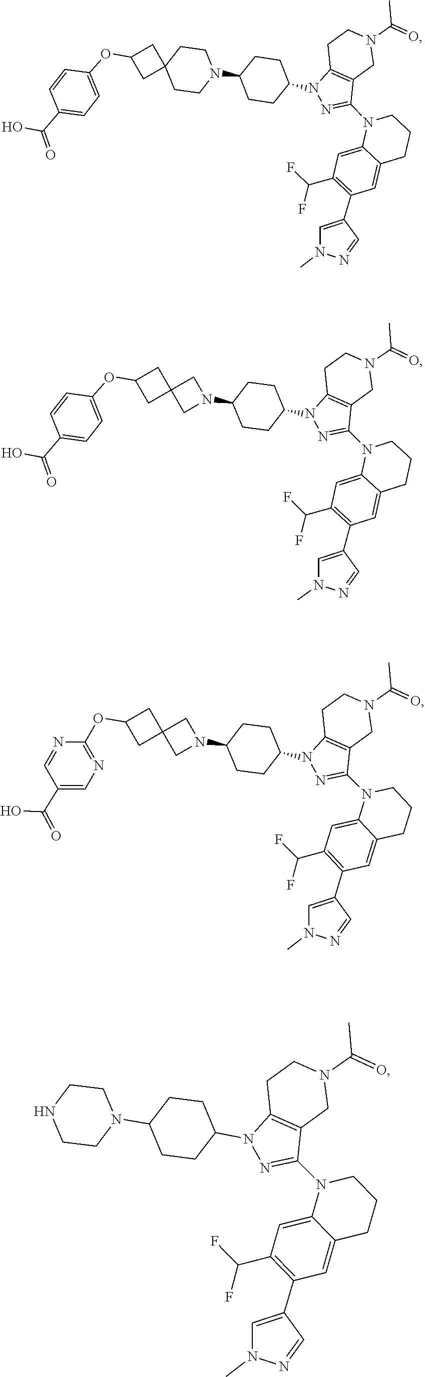 [SMILES: CC(=O)N1CCc2c(c(N3CCCc4cc(-c5cnn(C)c5)c(C(F)F)cc43)nn2[C@H]2CC[C@H](N3CCC4(CC3)CC(Oc3ccc(C(=O)O)cc3)C4)CC2)C1 ; CC(=O)N1CCc2c(c(N3CCCc4cc(-c5cnn(C)c5)c(C(F)F)cc43)nn2[C@H]2CC[C@H](N3CC4(CC(Oc5ccc(C(=O)O)cc5)C4)C3)CC2)C1 ; CC(=O)N1CCc2c(c(N3CCCc4cc(-c5cnn(C)c5)c(C(F)F)cc43)nn2[C@H]2CC[C@H](N3CC4(CC(Oc5ncc(C(=O)O)cn5)C4)C3)CC2)C1 ; CC(=O)N1CCc2c(c(N3CCCc4cc(-c5cnn(C)c5)c(C(F)F)cc43)nn2C2CCC(N3CCNCC3)CC2)C1]

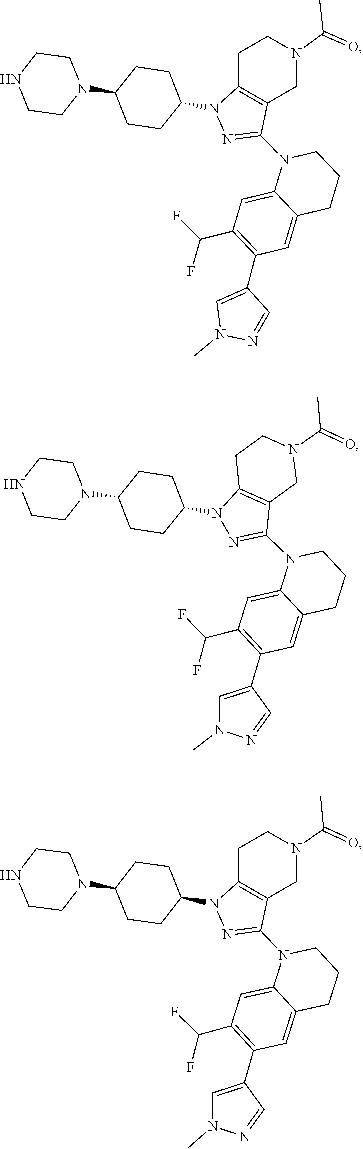 [SMILES: CC(=O)N1CCc2c(c(N3CCCc4cc(-c5cnn(C)c5)c(C(F)F)cc43)nn2[C@H]2CC[C@H](N3CCNCC3)CC2)C1 ; CC(=O)N1CCc2c(c(N3CCCc4cc(-c5cnn(C)c5)c(C(F)F)cc43)nn2[C@H]2CC[C@@H](N3CCNCC3)CC2)C1 ; CC(=O)N1CCc2c(c(N3CCCc4cc(-c5cnn(C)c5)c(C(F)F)cc43)nn2[C@H]2CC[C@@H](N3CCNCC3)CC2)C1]

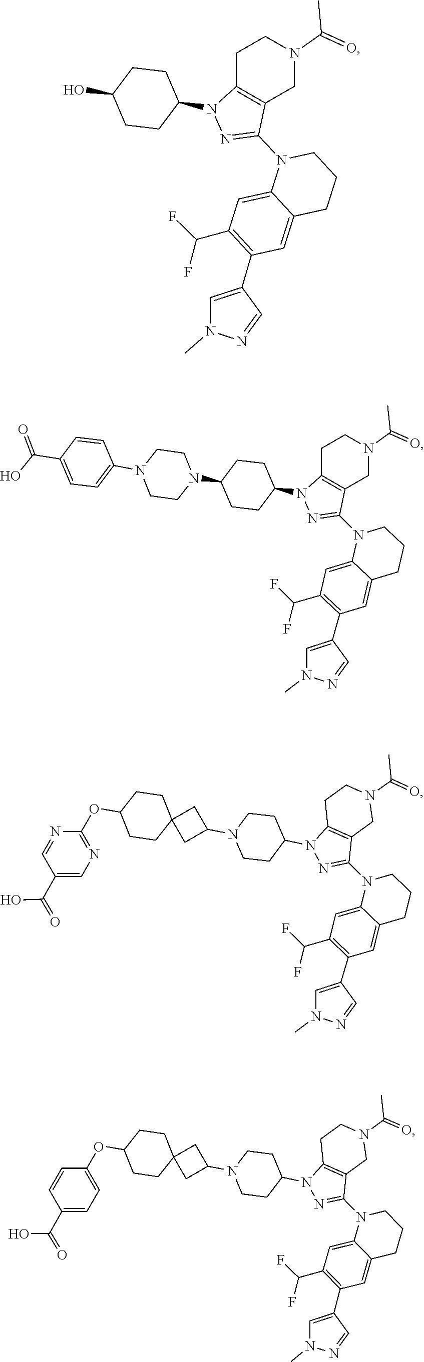 [SMILES: CC(=O)N1CCc2c(c(N3CCCc4cc(-c5cnn(C)c5)c(C(F)F)cc43)nn2[C@H]2CC[C@@H](O)CC2)C1 ; CC(=O)N1CCc2c(c(N3CCCc4cc(-c5cnn(C)c5)c(C(F)F)cc43)nn2[C@H]2CC[C@@H](N3CCN(c4ccc(C(=O)O)cc4)CC3)CC2)C1 ; CC(=O)N1CCc2c(c(N3CCCc4cc(-c5cnn(C)c5)c(C(F)F)cc43)nn2C2CCN(C3CC4(CCC(Oc5ncc(C(=O)O)cn5)CC4)C3)CC2)C1 ; CC(=O)N1CCc2c(c(N3CCCc4cc(-c5cnn(C)c5)c(C(F)F)cc43)nn2C2CCN(C3CC4(CCC(Oc5ccc(C(=O)O)cc5)CC4)C3)CC2)C1]

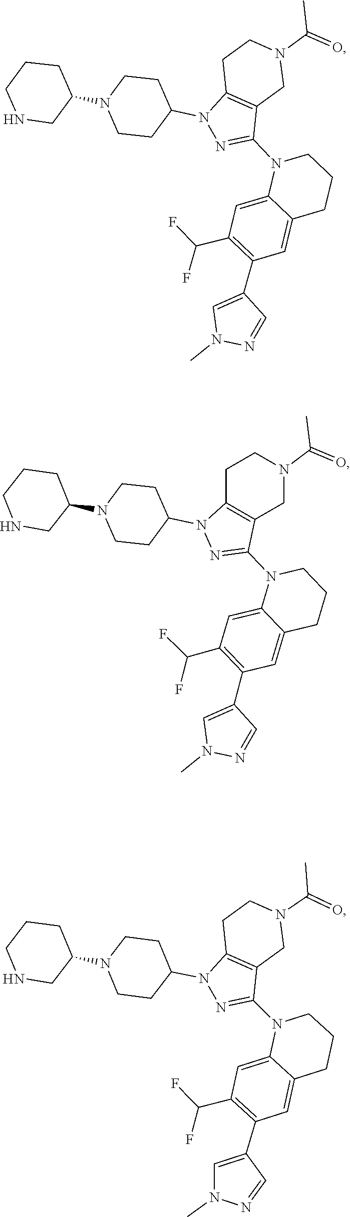 [SMILES: CC(=O)N1CCc2c(c(N3CCCc4cc(-c5cnn(C)c5)c(C(F)F)cc43)nn2C2CCN([C@H]3CCCNC3)CC2)C1 ; CC(=O)N1CCc2c(c(N3CCCc4cc(-c5cnn(C)c5)c(C(F)F)cc43)nn2C2CCN([C@@H]3CCCNC3)CC2)C1 ; CC(=O)N1CCc2c(c(N3CCCc4cc(-c5cnn(C)c5)c(C(F)F)cc43)nn2C2CCN([C@H]3CCCNC3)CC2)C1]

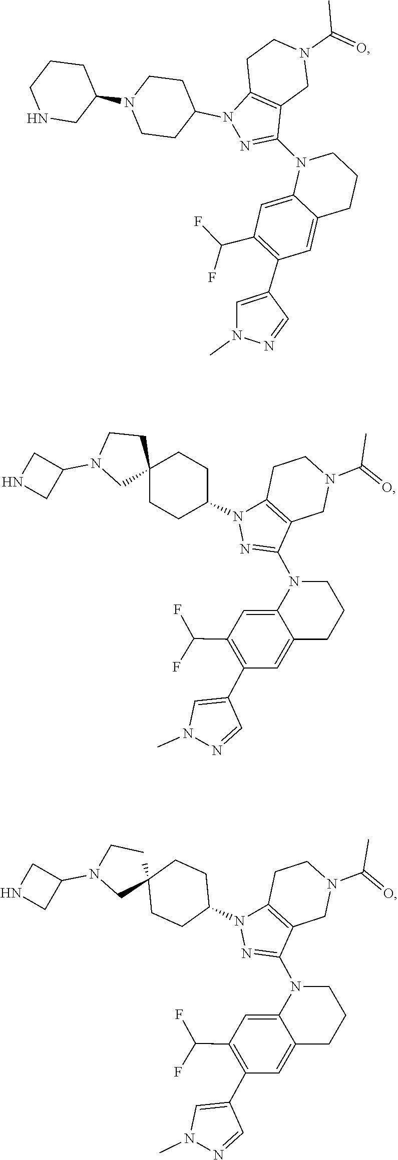 [SMILES: CC(=O)N1CCc2c(c(N3CCCc4cc(-c5cnn(C)c5)c(C(F)F)cc43)nn2C2CCN([C@@H]3CCCNC3)CC2)C1 ; CC(=O)N1CCc2c(c(N3CCCc4cc(-c5cnn(C)c5)c(C(F)F)cc43)nn2[C@H]2CC[C@]3(CCN(C4CNC4)C3)CC2)C1 ; CCN(C[C@]1(C)CC[C@@H](n2nc(N3CCCc4cc(-c5cnn(C)c5)c(C(F)F)cc43)c3c2CCN(C(C)=O)C3)CC1)C1CNC1]

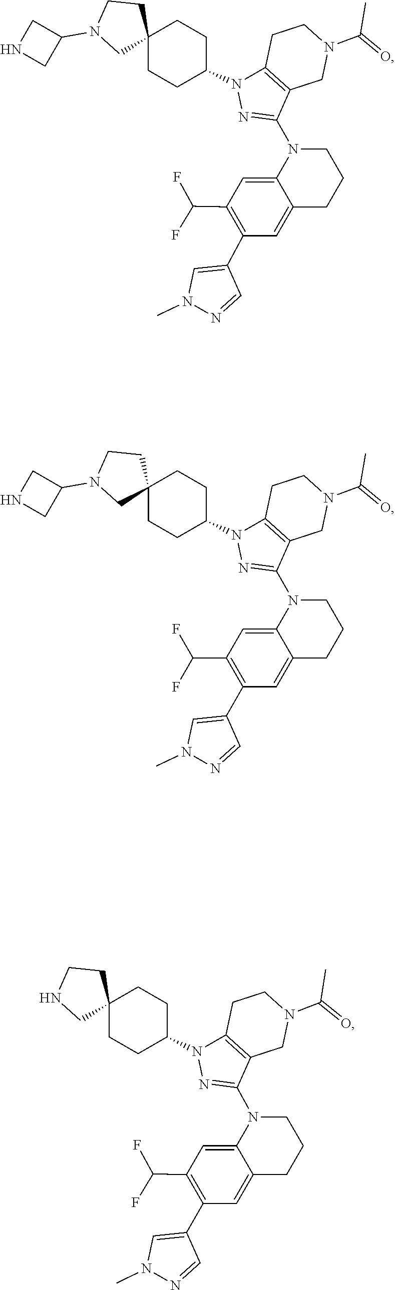 [SMILES: CC(=O)N1CCc2c(c(N3CCCc4cc(-c5cnn(C)c5)c(C(F)F)cc43)nn2[C@H]2CC[C@]3(CCN(C4CNC4)C3)CC2)C1 ; CC(=O)N1CCc2c(c(N3CCCc4cc(-c5cnn(C)c5)c(C(F)F)cc43)nn2[C@H]2CC[C@@]3(CCN(C4CNC4)C3)CC2)C1 ; CC(=O)N1CCc2c(c(N3CCCc4cc(-c5cnn(C)c5)c(C(F)F)cc43)nn2[C@H]2CC[C@]3(CCNC3)CC2)C1]

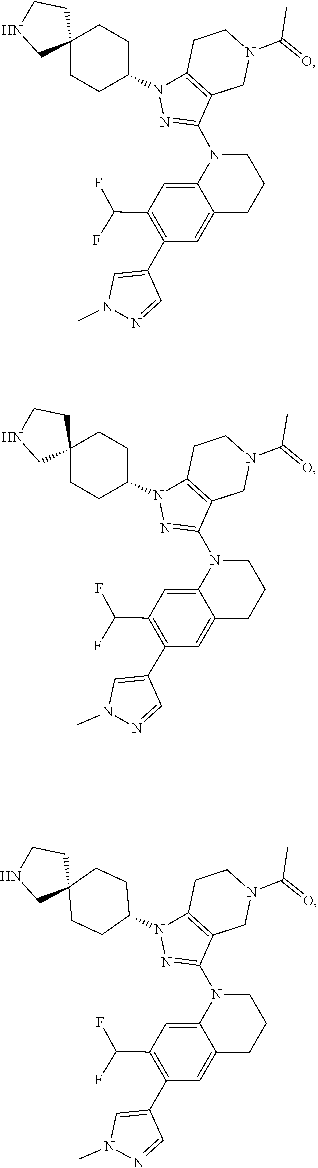 [SMILES: CC(=O)N1CCc2c(c(N3CCCc4cc(-c5cnn(C)c5)c(C(F)F)cc43)nn2[C@H]2CC[C@@]3(CCNC3)CC2)C1 ; CC(=O)N1CCc2c(c(N3CCCc4cc(-c5cnn(C)c5)c(C(F)F)cc43)nn2[C@H]2CC[C@]3(CCNC3)CC2)C1 ; CC(=O)N1CCc2c(c(N3CCCc4cc(-c5cnn(C)c5)c(C(F)F)cc43)nn2[C@H]2CC[C@@]3(CCNC3)CC2)C1]

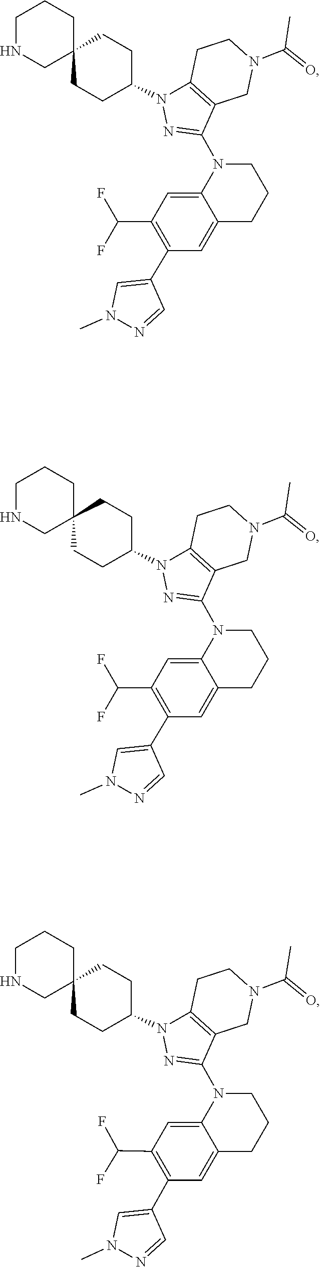 [SMILES: CC(=O)N1CCc2c(c(N3CCCc4cc(-c5cnn(C)c5)c(C(F)F)cc43)nn2[C@H]2CC[C@]3(CCCNC3)CC2)C1 ; CC(=O)N1CCc2c(c(N3CCCc4cc(-c5cnn(C)c5)c(C(F)F)cc43)nn2[C@H]2CC[C@@]3(CCCNC3)CC2)C1 ; CC(=O)N1CCc2c(c(N3CCCc4cc(-c5cnn(C)c5)c(C(F)F)cc43)nn2[C@H]2CC[C@]3(CCCNC3)CC2)C1]

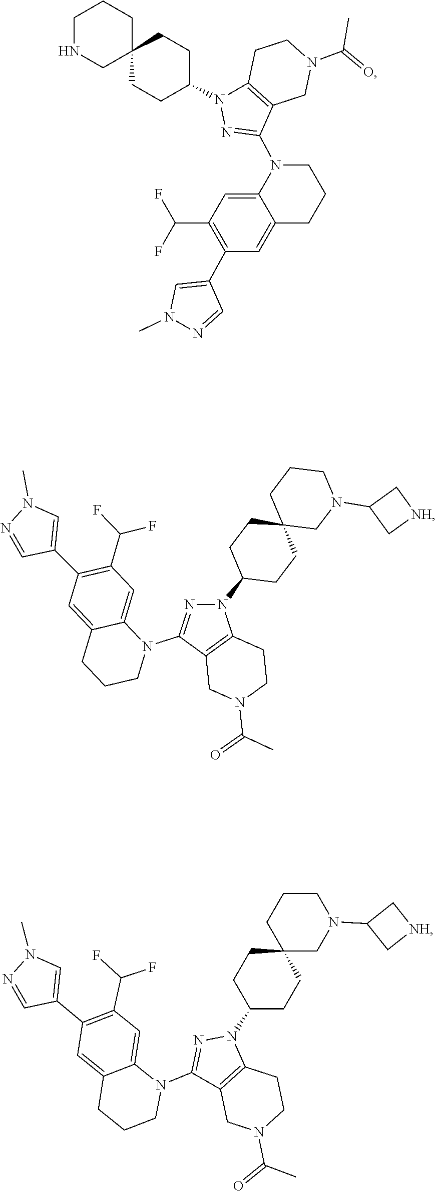 [SMILES: CC(=O)N1CCc2c(c(N3CCCc4cc(-c5cnn(C)c5)c(C(F)F)cc43)nn2[C@H]2CC[C@@]3(CCCNC3)CC2)C1 ; CC(=O)N1CCc2c(c(N3CCCc4cc(-c5cnn(C)c5)c(C(F)F)cc43)nn2[C@H]2CC[C@]3(CCCN(C4CNC4)C3)CC2)C1 ; CC(=O)N1CCc2c(c(N3CCCc4cc(-c5cnn(C)c5)c(C(F)F)cc43)nn2[C@H]2CC[C@@]3(CCCN(C4CNC4)C3)CC2)C1]

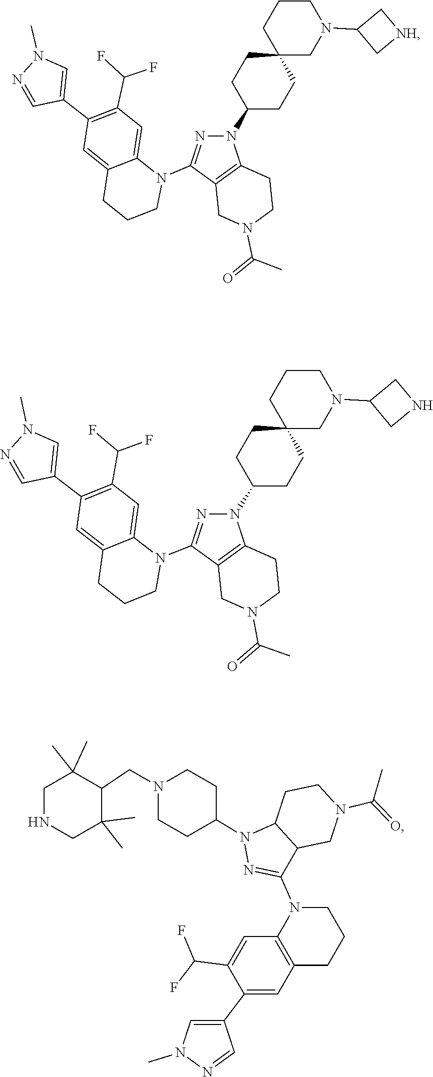 [SMILES: CC(=O)N1CCc2c(c(N3CCCc4cc(-c5cnn(C)c5)c(C(F)F)cc43)nn2[C@H]2CC[C@]3(CCCN(C4CNC4)C3)CC2)C1 ; CC(=O)N1CCc2c(c(N3CCCc4cc(-c5cnn(C)c5)c(C(F)F)cc43)nn2[C@H]2CC[C@@]3(CCCN(C4CNC4)C3)CC2)C1 ; CC(=O)N1CCC2C(C1)C(N1CCCc3cc(-c4cnn(C)c4)c(C(F)F)cc31)=NN2C1CCN(CC2C(C)(C)CNCC2(C)C)CC1]

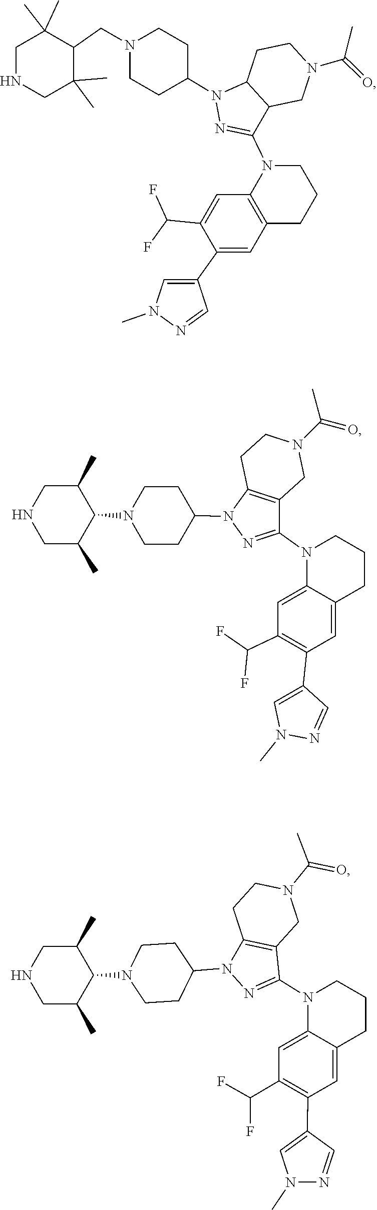 [SMILES: CC(=O)N1CCC2C(C1)C(N1CCCc3cc(-c4cnn(C)c4)c(C(F)F)cc31)=NN2C1CCN(CC2C(C)(C)CNCC2(C)C)CC1 ; CC(=O)N1CCc2c(c(N3CCCc4cc(-c5cnn(C)c5)c(C(F)F)cc43)nn2C2CCN([C@H]3[C@H](C)CNC[C@@H]3C)CC2)C1 ; CC(=O)N1CCc2c(c(N3CCCc4cc(-c5cnn(C)c5)c(C(F)F)cc43)nn2C2CCN([C@H]3[C@H](C)CNC[C@@H]3C)CC2)C1]

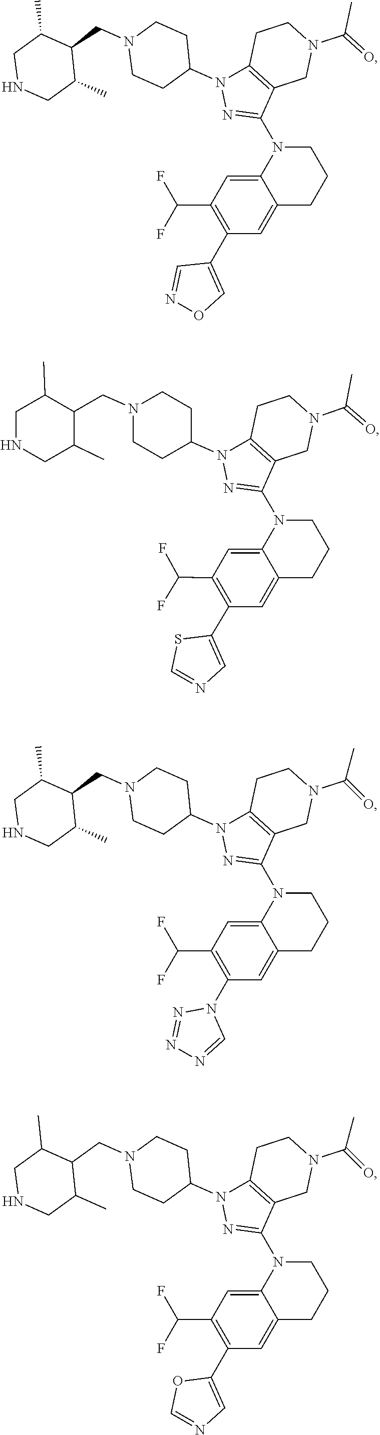 [SMILES: CC(=O)N1CCc2c(c(N3CCCc4cc(-c5cnoc5)c(C(F)F)cc43)nn2C2CCN(C[C@H]3[C@H](C)CNC[C@@H]3C)CC2)C1 ; CC(=O)N1CCc2c(c(N3CCCc4cc(-c5cncs5)c(C(F)F)cc43)nn2C2CCN(CC3C(C)CNCC3C)CC2)C1 ; CC(=O)N1CCc2c(c(N3CCCc4cc(-n5cnnn5)c(C(F)F)cc43)nn2C2CCN(C[C@H]3[C@H](C)CNC[C@@H]3C)CC2)C1 ; CC(=O)N1CCc2c(c(N3CCCc4cc(-c5cnco5)c(C(F)F)cc43)nn2C2CCN(CC3C(C)CNCC3C)CC2)C1]

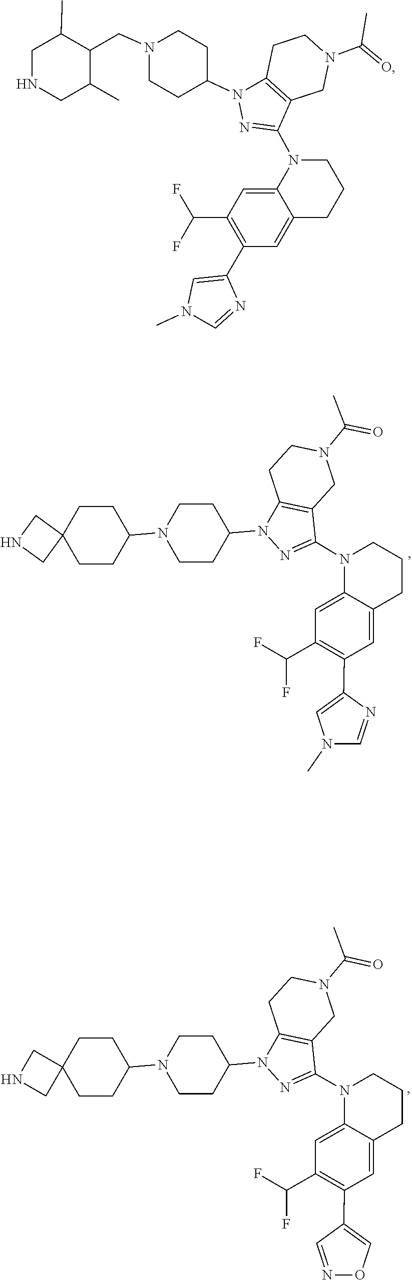 [SMILES: CC(=O)N1CCc2c(c(N3CCCc4cc(-c5cn(C)cn5)c(C(F)F)cc43)nn2C2CCN(CC3C(C)CNCC3C)CC2)C1 ; CC(=O)N1CCc2c(c(N3CCCc4cc(-c5cn(C)cn5)c(C(F)F)cc43)nn2C2CCN(C3CCC4(CC3)CNC4)CC2)C1 ; CC(=O)N1CCc2c(c(N3CCCc4cc(-c5cnoc5)c(C(F)F)cc43)nn2C2CCN(C3CCC4(CC3)CNC4)CC2)C1]

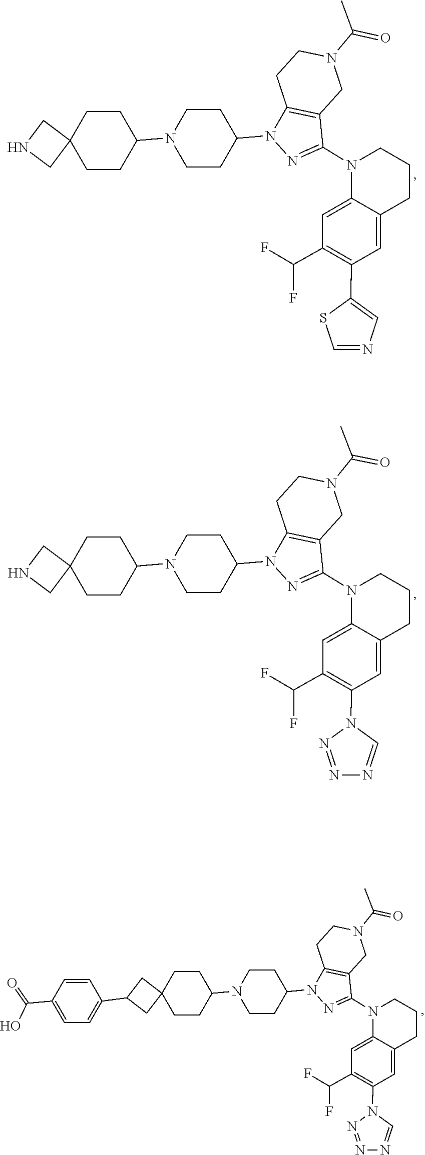 [SMILES: CC(=O)N1CCc2c(c(N3CCCc4cc(-c5cncs5)c(C(F)F)cc43)nn2C2CCN(C3CCC4(CC3)CNC4)CC2)C1 ; CC(=O)N1CCc2c(c(N3CCCc4cc(-n5cnnn5)c(C(F)F)cc43)nn2C2CCN(C3CCC4(CC3)CNC4)CC2)C1 ; CC(=O)N1CCc2c(c(N3CCCc4cc(-n5cnnn5)c(C(F)F)cc43)nn2C2CCN(C3CCC4(CC3)CC(c3ccc(C(=O)O)cc3)C4)CC2)C1]

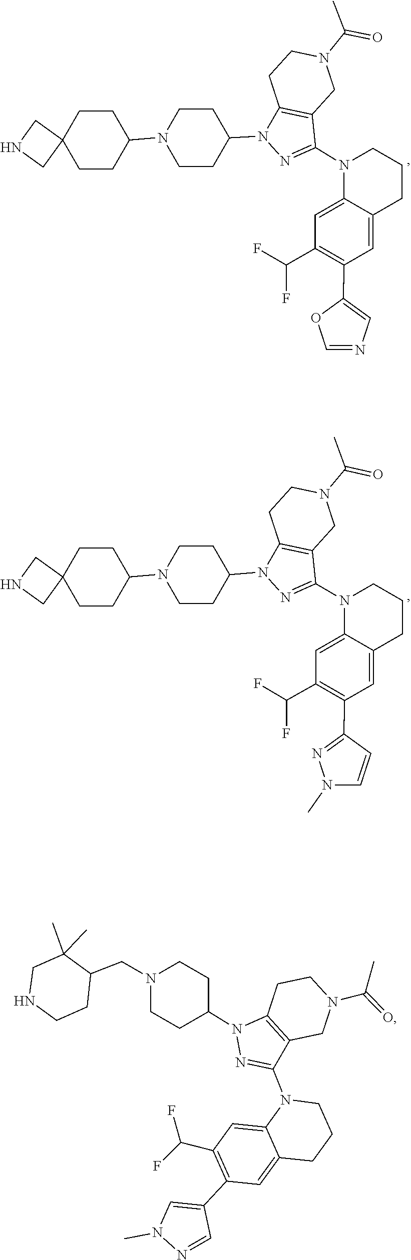 [SMILES: CC(=O)N1CCc2c(c(N3CCCc4cc(-c5cnco5)c(C(F)F)cc43)nn2C2CCN(C3CCC4(CC3)CNC4)CC2)C1 ; CC(=O)N1CCc2c(c(N3CCCc4cc(-c5ccn(C)n5)c(C(F)F)cc43)nn2C2CCN(C3CCC4(CC3)CNC4)CC2)C1 ; CC(=O)N1CCc2c(c(N3CCCc4cc(-c5cnn(C)c5)c(C(F)F)cc43)nn2C2CCN(CC3CCNCC3(C)C)CC2)C1]

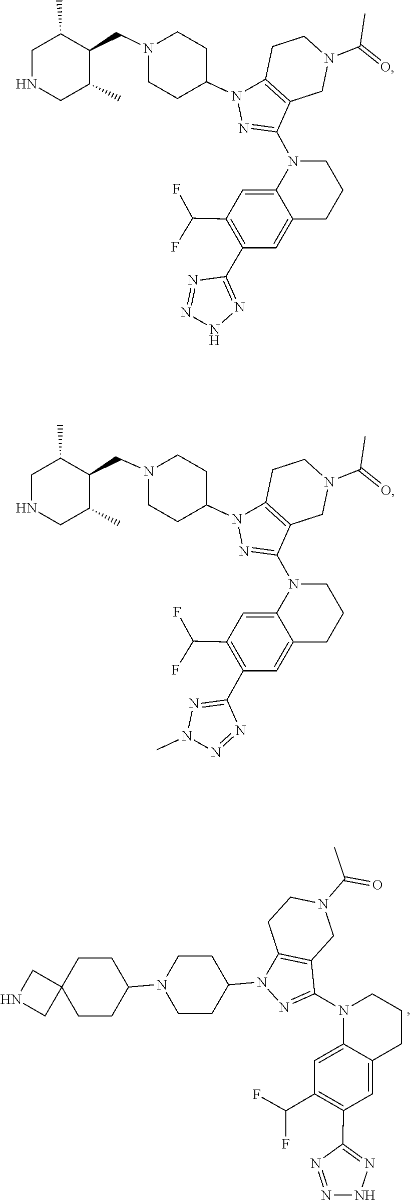 [SMILES: CC(=O)N1CCc2c(c(N3CCCc4cc(-c5nn[nH]n5)c(C(F)F)cc43)nn2C2CCN(C[C@H]3[C@H](C)CNC[C@@H]3C)CC2)C1 ; CC(=O)N1CCc2c(c(N3CCCc4cc(-c5nnn(C)n5)c(C(F)F)cc43)nn2C2CCN(C[C@H]3[C@H](C)CNC[C@@H]3C)CC2)C1 ; CC(=O)N1CCc2c(c(N3CCCc4cc(-c5nn[nH]n5)c(C(F)F)cc43)nn2C2CCN(C3CCC4(CC3)CNC4)CC2)C1]

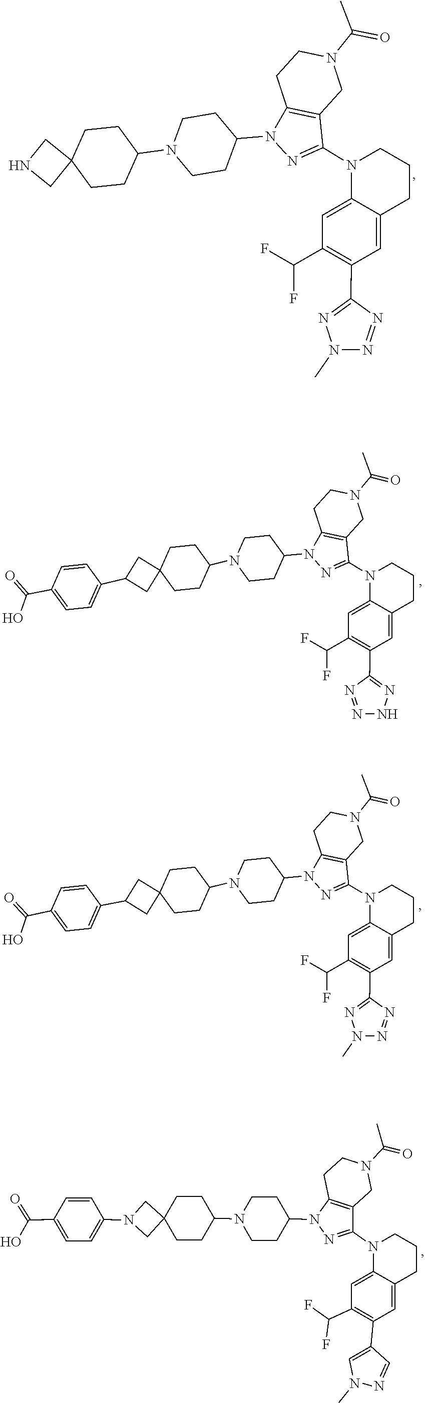 [SMILES: CC(=O)N1CCc2c(c(N3CCCc4cc(-c5nnn(C)n5)c(C(F)F)cc43)nn2C2CCN(C3CCC4(CC3)CNC4)CC2)C1 ; CC(=O)N1CCc2c(c(N3CCCc4cc(-c5nn[nH]n5)c(C(F)F)cc43)nn2C2CCN(C3CCC4(CC3)CC(c3ccc(C(=O)O)cc3)C4)CC2)C1 ; CC(=O)N1CCc2c(c(N3CCCc4cc(-c5nnn(C)n5)c(C(F)F)cc43)nn2C2CCN(C3CCC4(CC3)CC(c3ccc(C(=O)O)cc3)C4)CC2)C1 ; CC(=O)N1CCc2c(c(N3CCCc4cc(-c5cnn(C)c5)c(C(F)F)cc43)nn2C2CCN(C3CCC4(CC3)CN(c3ccc(C(=O)O)cc3)C4)CC2)C1]

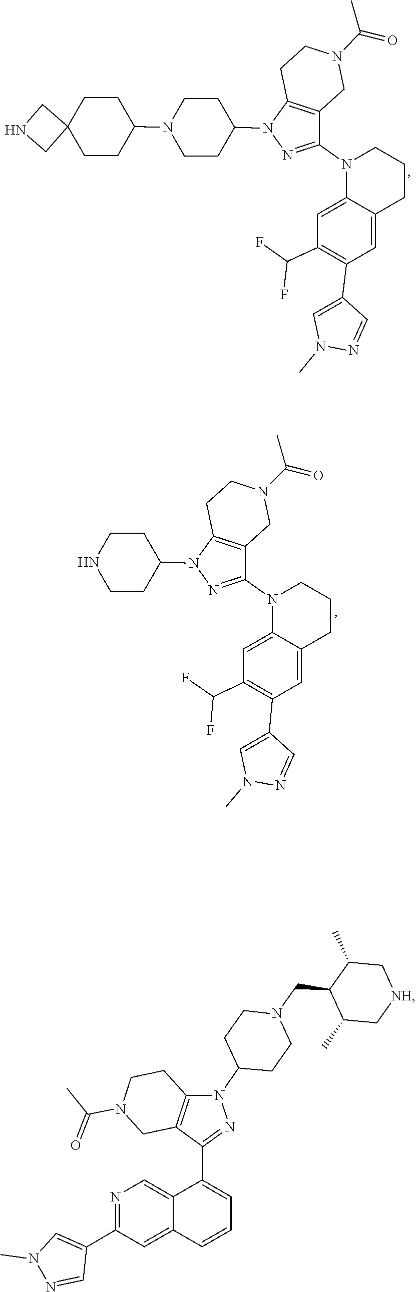 [SMILES: CC(=O)N1CCc2c(c(N3CCCc4cc(-c5cnn(C)c5)c(C(F)F)cc43)nn2C2CCN(C3CCC4(CC3)CNC4)CC2)C1 ; CC(=O)N1CCc2c(c(N3CCCc4cc(-c5cnn(C)c5)c(C(F)F)cc43)nn2C2CCNCC2)C1 ; CC(=O)N1CCc2c(c(-c3cccc4cc(-c5cnn(C)c5)ncc34)nn2C2CCN(C[C@H]3[C@H](C)CNC[C@@H]3C)CC2)C1]

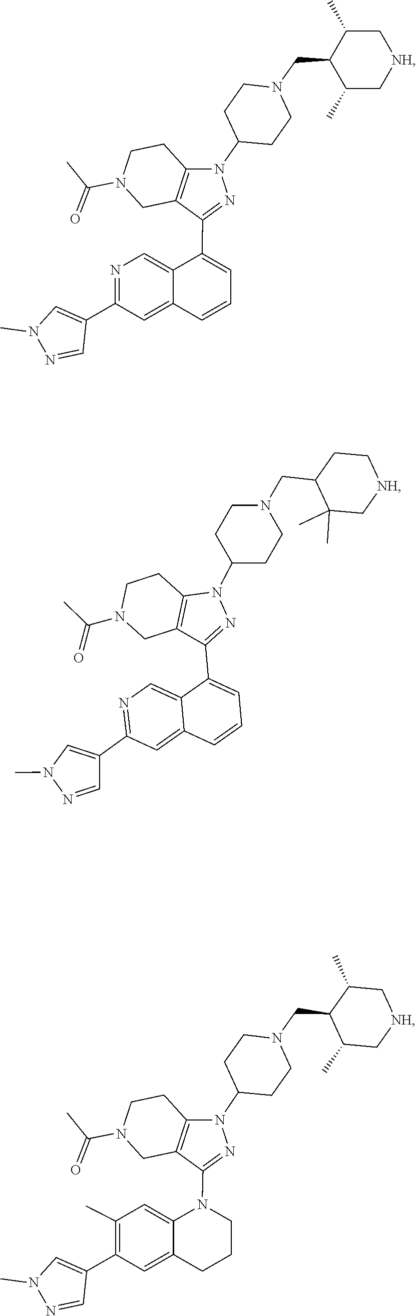 [SMILES: CC(=O)N1CCc2c(c(-c3cccc4cc(-c5cnn(C)c5)ncc34)nn2C2CCN(C[C@H]3[C@H](C)CNC[C@@H]3C)CC2)C1 ; CC(=O)N1CCc2c(c(-c3cccc4cc(-c5cnn(C)c5)ncc34)nn2C2CCN(CC3CCNCC3(C)C)CC2)C1 ; CC(=O)N1CCc2c(c(N3CCCc4cc(-c5cnn(C)c5)c(C)cc43)nn2C2CCN(C[C@H]3[C@H](C)CNC[C@@H]3C)CC2)C1]

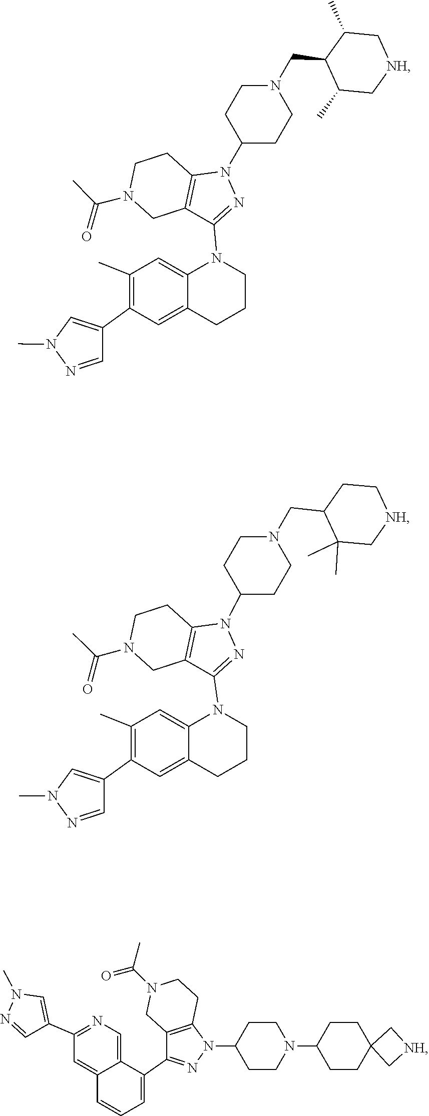 [SMILES: CC(=O)N1CCc2c(c(N3CCCc4cc(-c5cnn(C)c5)c(C)cc43)nn2C2CCN(C[C@H]3[C@H](C)CNC[C@@H]3C)CC2)C1 ; CC(=O)N1CCc2c(c(N3CCCc4cc(-c5cnn(C)c5)c(C)cc43)nn2C2CCN(CC3CCNCC3(C)C)CC2)C1 ; CC(=O)N1CCc2c(c(-c3cccc4cc(-c5cnn(C)c5)ncc34)nn2C2CCN(C3CCC4(CC3)CNC4)CC2)C1]

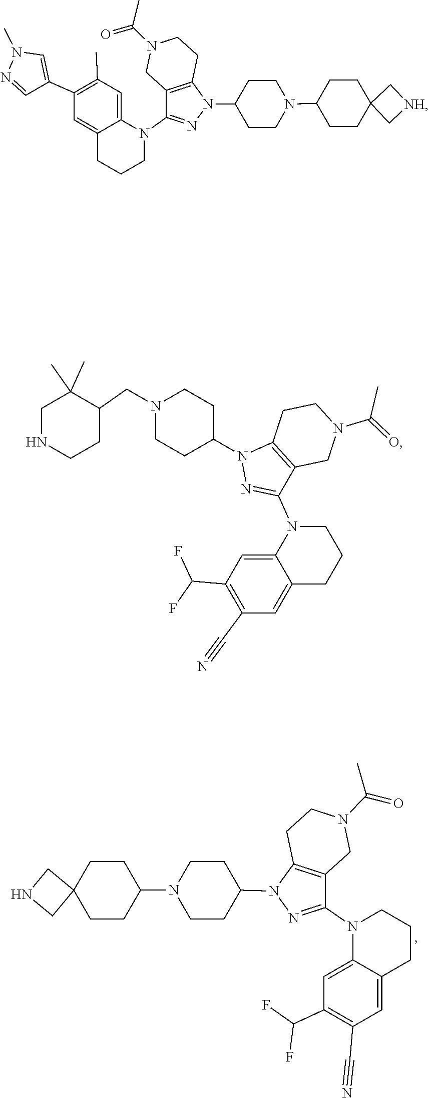 [SMILES: CC(=O)N1CCc2c(c(N3CCCc4cc(-c5cnn(C)c5)c(C)cc43)nn2C2CCN(C3CCC4(CC3)CNC4)CC2)C1 ; CC(=O)N1CCc2c(c(N3CCCc4cc(C#N)c(C(F)F)cc43)nn2C2CCN(CC3CCNCC3(C)C)CC2)C1 ; CC(=O)N1CCc2c(c(N3CCCc4cc(C#N)c(C(F)F)cc43)nn2C2CCN(C3CCC4(CC3)CNC4)CC2)C1]

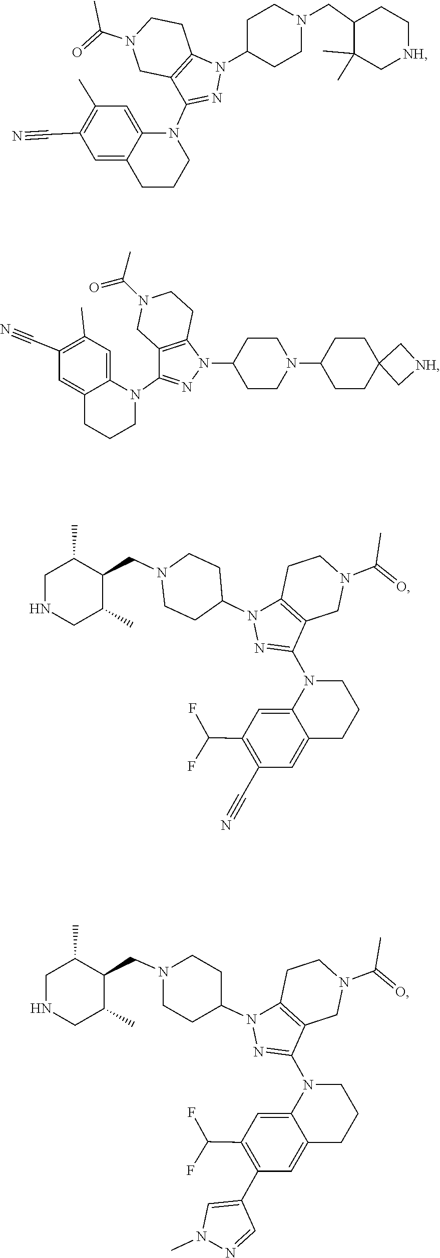 [SMILES: CC(=O)N1CCc2c(c(N3CCCc4cc(C#N)c(C)cc43)nn2C2CCN(CC3CCNCC3(C)C)CC2)C1 ; CC(=O)N1CCc2c(c(N3CCCc4cc(C#N)c(C)cc43)nn2C2CCN(C3CCC4(CC3)CNC4)CC2)C1 ; CC(=O)N1CCc2c(c(N3CCCc4cc(C#N)c(C(F)F)cc43)nn2C2CCN(C[C@H]3[C@H](C)CNC[C@@H]3C)CC2)C1 ; CC(=O)N1CCc2c(c(N3CCCc4cc(-c5cnn(C)c5)c(C(F)F)cc43)nn2C2CCN(C[C@H]3[C@H](C)CNC[C@@H]3C)CC2)C1]

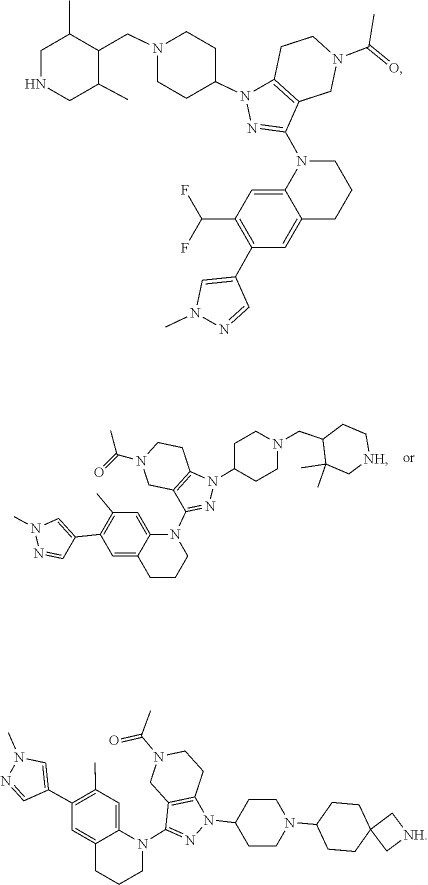 [SMILES: CC(=O)N1CCc2c(c(N3CCCc4cc(-c5cnn(C)c5)c(C(F)F)cc43)nn2C2CCN(CC3C(C)CNCC3C)CC2)C1 ; CC(=O)N1CCc2c(c(N3CCCc4cc(-c5cnn(C)c5)c(C)cc43)nn2C2CCN(CC3CCNCC3(C)C)CC2)C1 ; "or" ; CC(=O)N1CCc2c(c(N3CCCc4cc(-c5cnn(C)c5)c(C)cc43)nn2C2CCN(C3CCC4(CC3)CNC4)CC2)C1]

Embodiment 125. A compound, or a pharmaceutically acceptable salt or solvate thereof, that has one of the following structures:

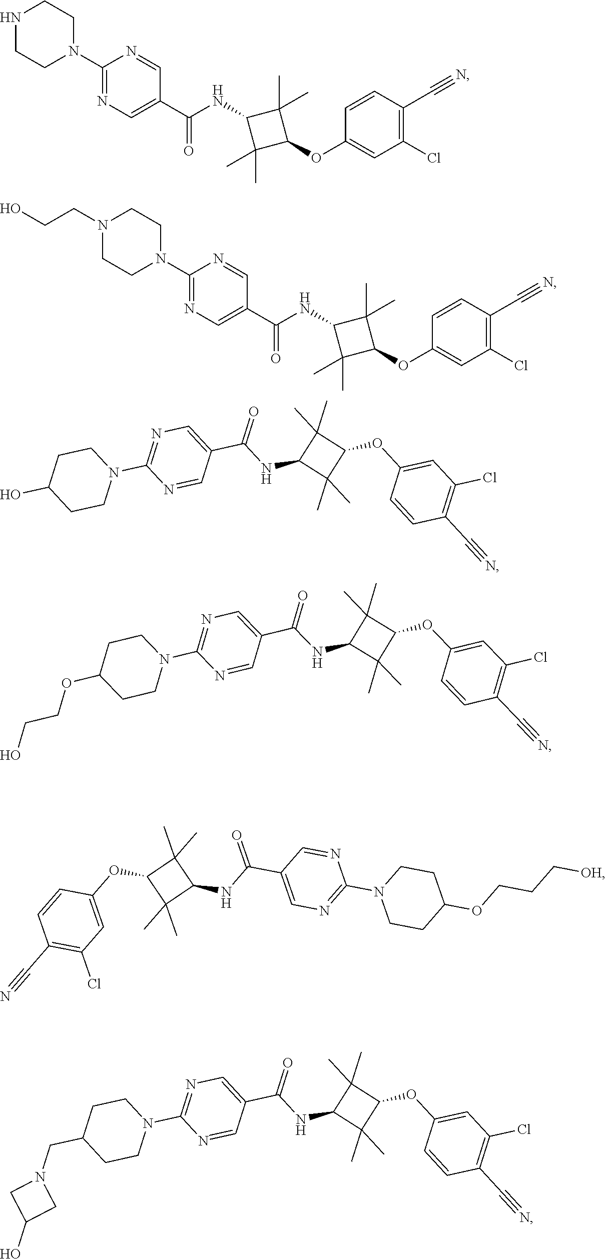

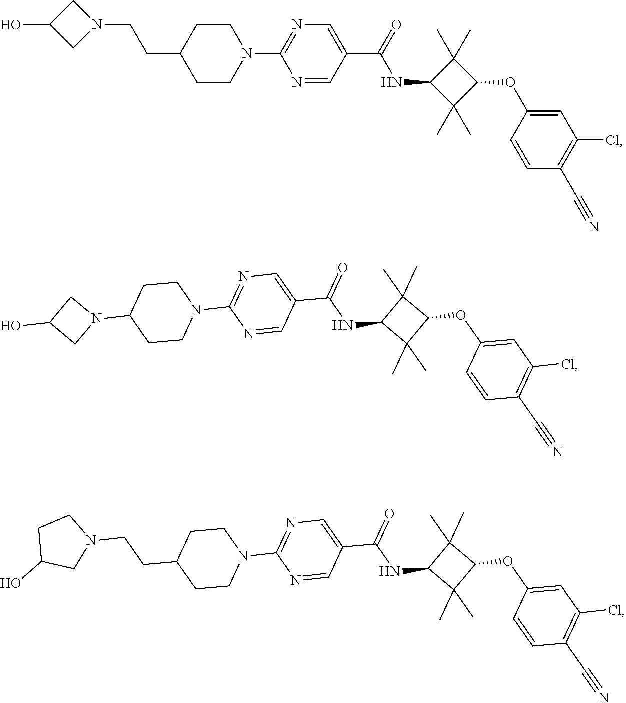 [SMILES: CC1(C)[C@H](NC(=O)c2cnc(N3CCC(CCN4CC(O)C4)CC3)nc2)C(C)(C)[C@H]1Oc1ccc(C#N)c(Cl)c1 ; CC1(C)[C@H](NC(=O)c2cnc(N3CCC(N4CC(O)C4)CC3)nc2)C(C)(C)[C@H]1Oc1ccc(C#N)c(Cl)c1 ; CC1(C)[C@H](NC(=O)c2cnc(N3CCC(CCN4CCC(O)C4)CC3)nc2)C(C)(C)[C@H]1Oc1ccc(C#N)c(Cl)c1]

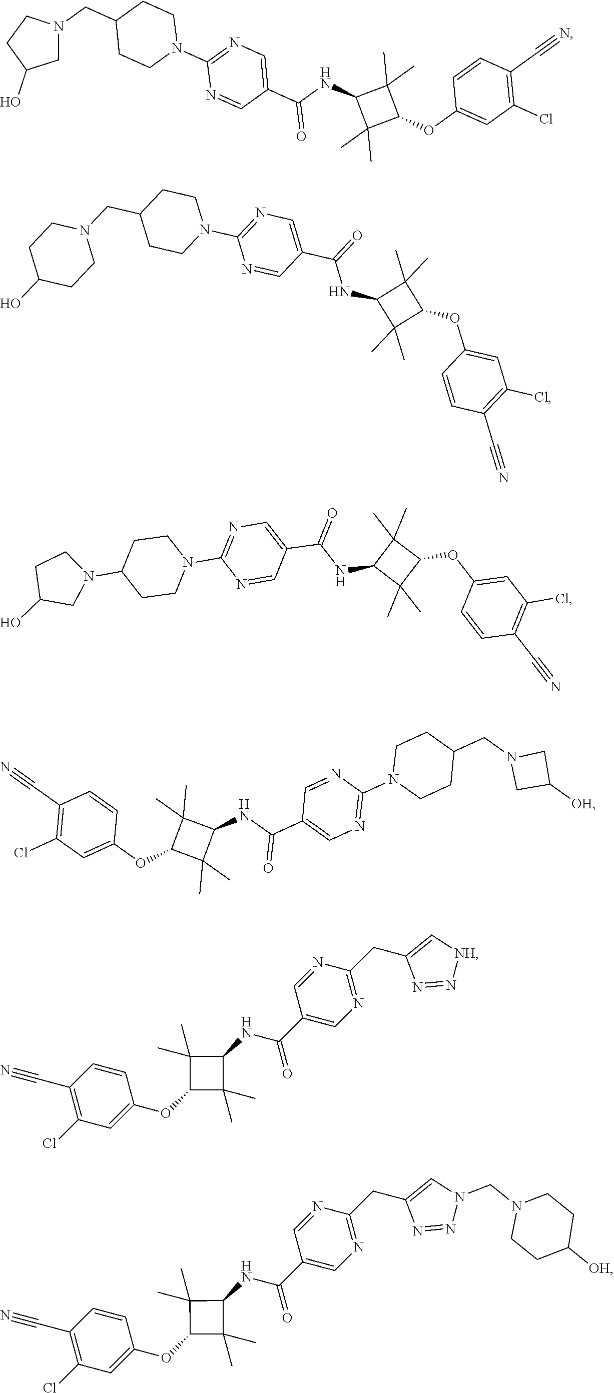 [SMILES: CC1(C)[C@H](NC(=O)c2cnc(N3CCC(CN4CCC(O)C4)CC3)nc2)C(C)(C)[C@H]1Oc1ccc(C#N)c(Cl)c1 ; CC1(C)[C@H](NC(=O)c2cnc(N3CCC(CN4CCC(O)CC4)CC3)nc2)C(C)(C)[C@H]1Oc1ccc(C#N)c(Cl)c1 ; CC1(C)[C@H](NC(=O)c2cnc(N3CCC(N4CCC(O)C4)CC3)nc2)C(C)(C)[C@H]1Oc1ccc(C#N)c(Cl)c1 ; CC1(C)[C@H](NC(=O)c2cnc(N3CCC(CN4CC(O)C4)CC3)nc2)C(C)(C)[C@H]1Oc1ccc(C#N)c(Cl)c1 ; CC1(C)[C@H](NC(=O)c2cnc(Cc3c[nH]nn3)nc2)C(C)(C)[C@H]1Oc1ccc(C#N)c(Cl)c1 ; CC1(C)[C@H](NC(=O)c2cnc(Cc3cn(CN4CCC(O)CC4)nn3)nc2)C(C)(C)[C@H]1Oc1ccc(C#N)c(Cl)c1]

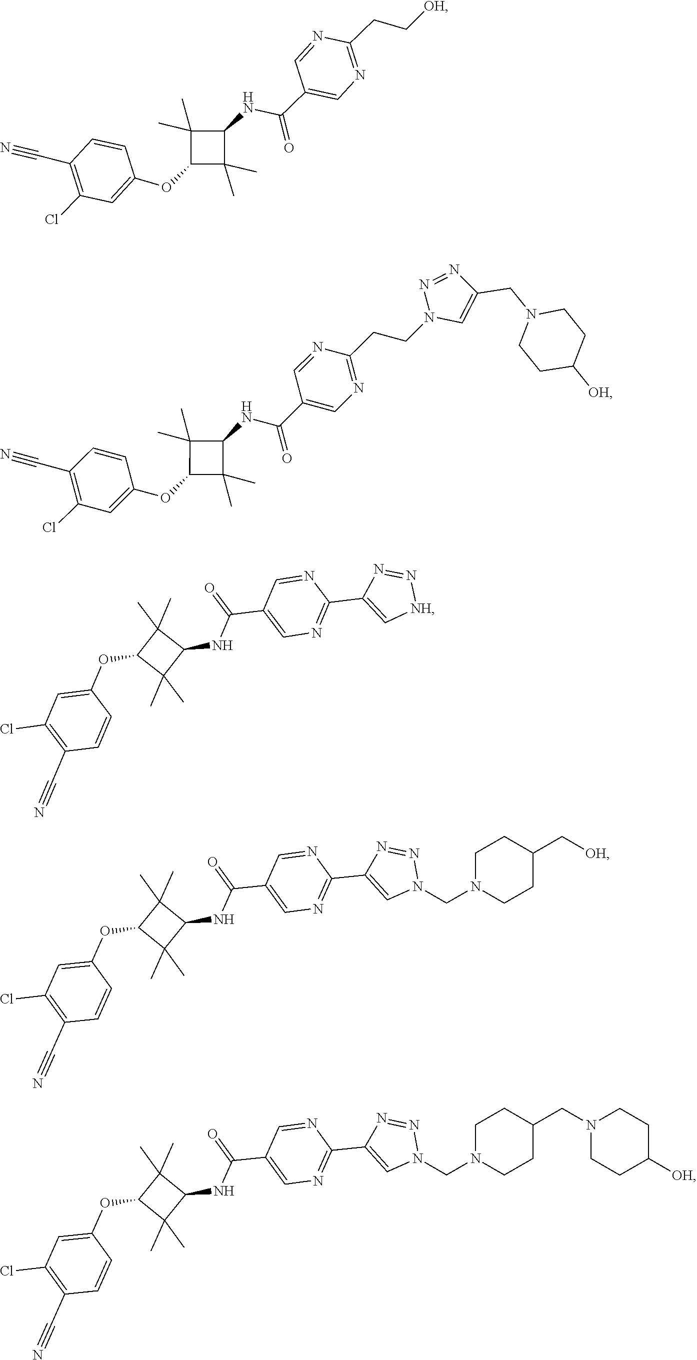 [SMILES: CC1(C)[C@H](NC(=O)c2cnc(CCO)nc2)C(C)(C)[C@H]1Oc1ccc(C#N)c(Cl)c1 ; CC1(C)[C@H](NC(=O)c2cnc(CCn3cc(CN4CCC(O)CC4)nn3)nc2)C(C)(C)[C@H]1Oc1ccc(C#N)c(Cl)c1 ; CC1(C)[C@H](NC(=O)c2cnc(-c3c[nH]nn3)nc2)C(C)(C)[C@H]1Oc1ccc(C#N)c(Cl)c1 ; CC1(C)[C@H](NC(=O)c2cnc(-c3cn(CN4CCC(CO)CC4)nn3)nc2)C(C)(C)[C@H]1Oc1ccc(C#N)c(Cl)c1 ; CC1(C)[C@H](NC(=O)c2cnc(-c3cn(CN4CCC(CN5CCC(O)CC5)CC4)nn3)nc2)C(C)(C)[C@H]1Oc1ccc(C#N)c(Cl)c1]

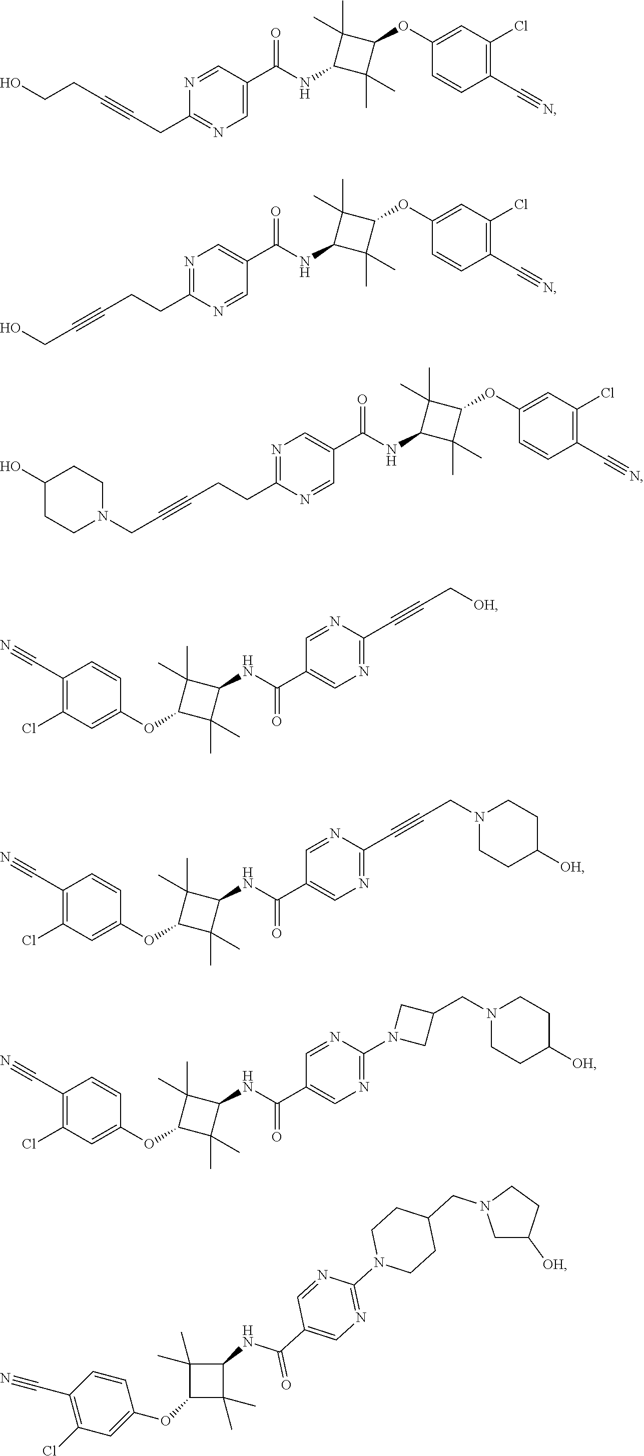

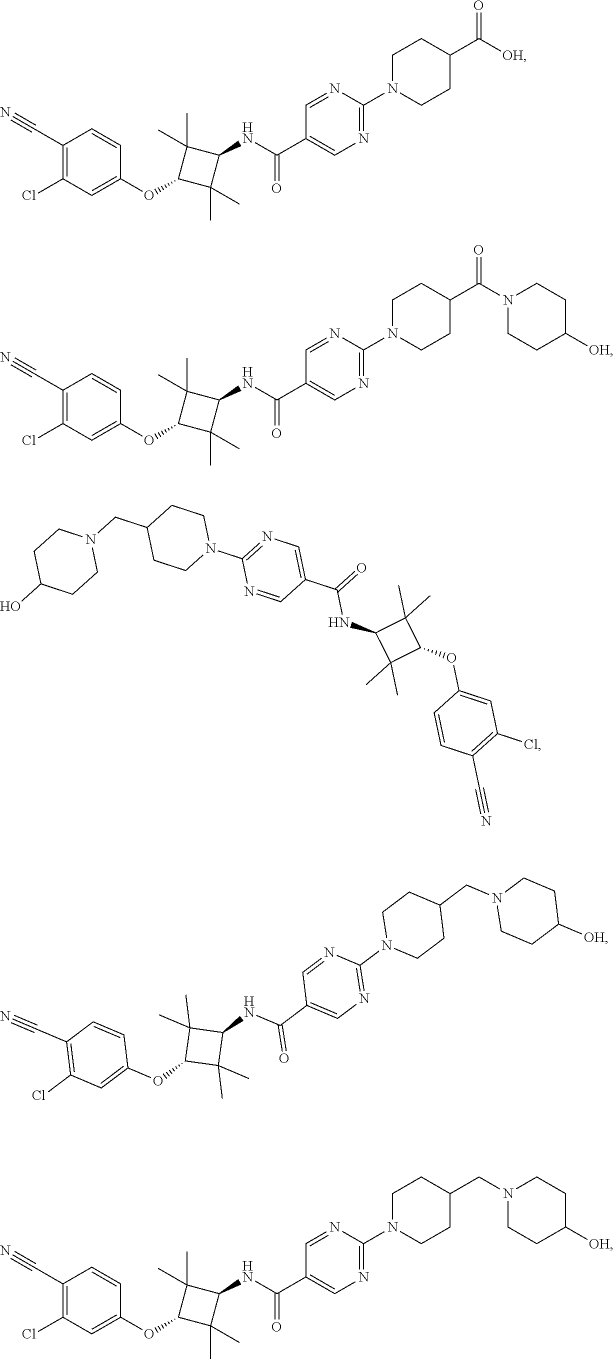 [SMILES: CC1(C)[C@H](NC(=O)c2cnc(N3CCC(C(=O)O)CC3)nc2)C(C)(C)[C@H]1Oc1ccc(C#N)c(Cl)c1 ; CC1(C)[C@H](NC(=O)c2cnc(N3CCC(C(=O)N4CCC(O)CC4)CC3)nc2)C(C)(C)[C@H]1Oc1ccc(C#N)c(Cl)c1 ; CC1(C)[C@H](NC(=O)c2cnc(N3CCC(CN4CCC(O)CC4)CC3)nc2)C(C)(C)[C@H]1Oc1ccc(C#N)c(Cl)c1 ; CC1(C)[C@H](NC(=O)c2cnc(N3CCC(CN4CCC(O)CC4)CC3)nc2)C(C)(C)[C@H]1Oc1ccc(C#N)c(Cl)c1 ; CC1(C)[C@H](NC(=O)c2cnc(N3CCC(CN4CCC(O)CC4)CC3)nc2)C(C)(C)[C@H]1Oc1ccc(C#N)c(Cl)c1]

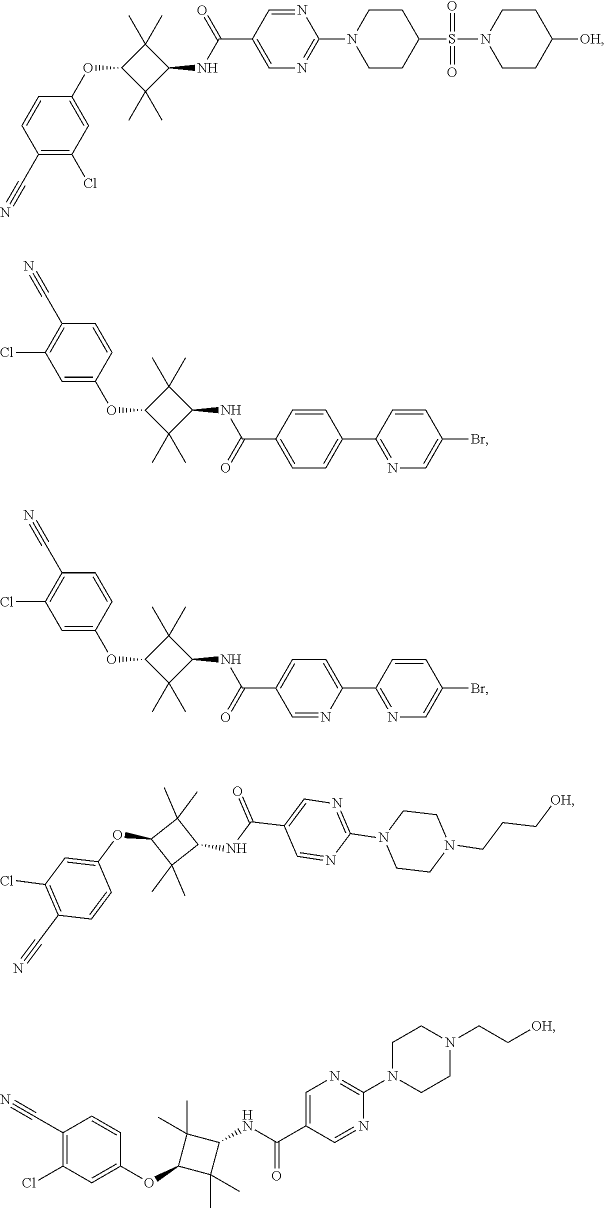 [SMILES: CC1(C)[C@H](NC(=O)c2cnc(N3CCC(S(=O)(=O)N4CCC(O)CC4)CC3)nc2)C(C)(C)[C@H]1Oc1ccc(C#N)c(Cl)c1 ; CC1(C)[C@H](NC(=O)c2ccc(-c3ccc(Br)cn3)cc2)C(C)(C)[C@H]1Oc1ccc(C#N)c(Cl)c1 ; CC1(C)[C@H](NC(=O)c2ccc(-c3ccc(Br)cn3)nc2)C(C)(C)[C@H]1Oc1ccc(C#N)c(Cl)c1 ; CC1(C)[C@H](NC(=O)c2cnc(N3CCN(CCCO)CC3)nc2)C(C)(C)[C@H]1Oc1ccc(C#N)c(Cl)c1 ; CC1(C)[C@H](NC(=O)c2cnc(N3CCN(CCO)CC3)nc2)C(C)(C)[C@H]1Oc1ccc(C#N)c(Cl)c1]

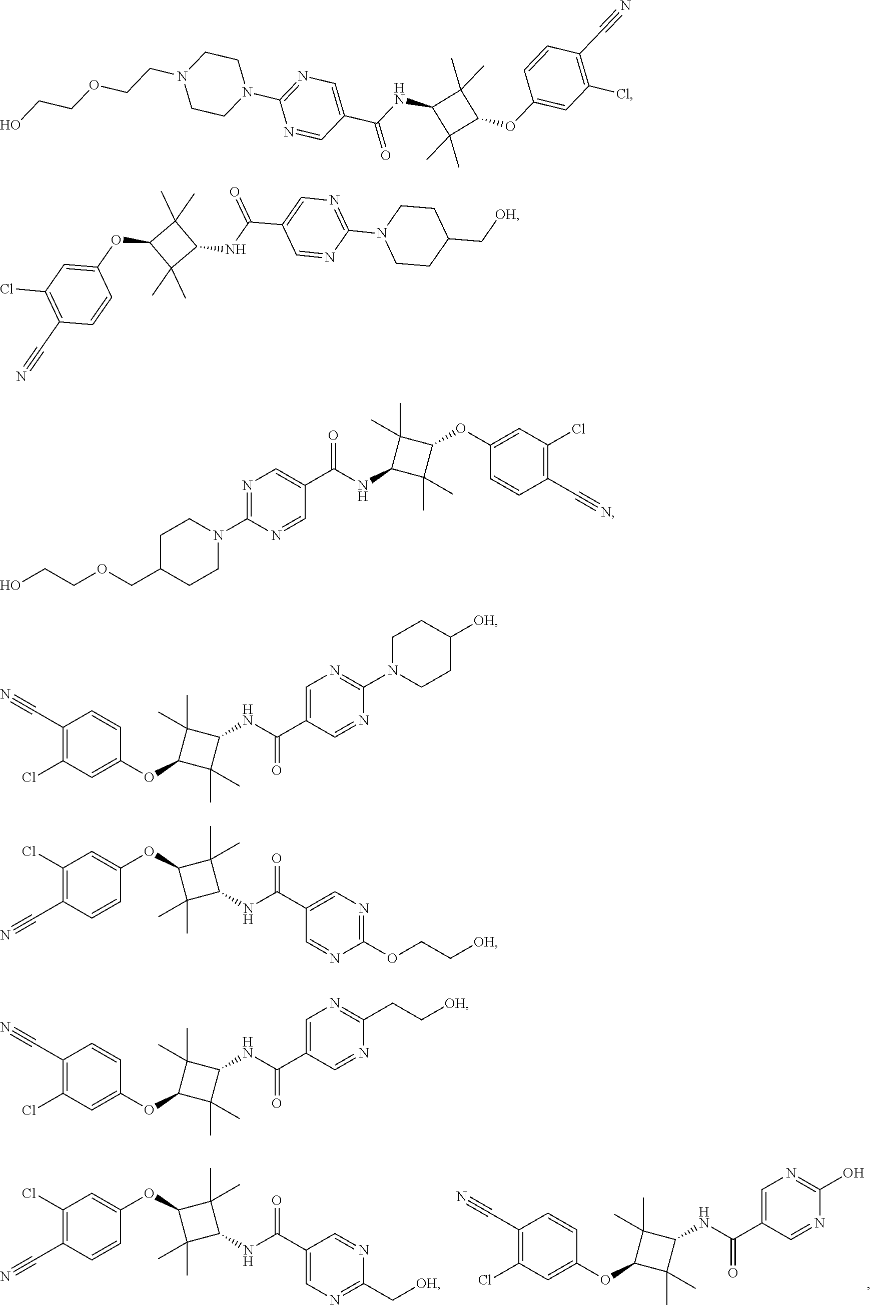

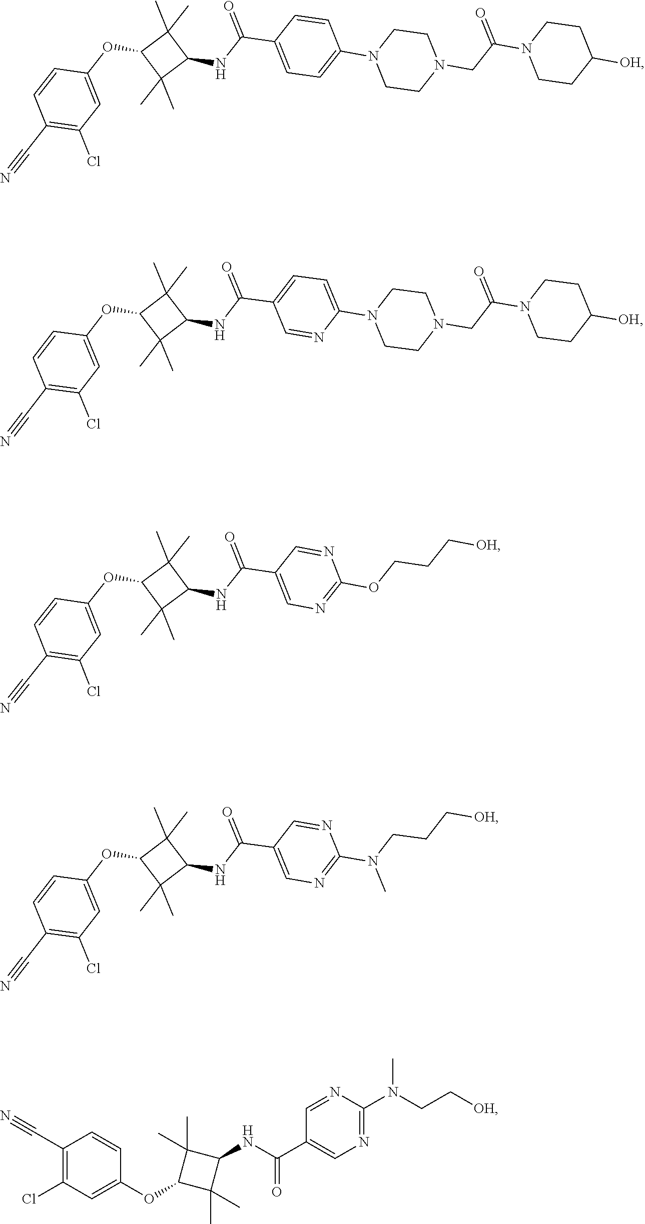 [SMILES: CC1(C)[C@H](NC(=O)c2ccc(N3CCN(CC(=O)N4CCC(O)CC4)CC3)cc2)C(C)(C)[C@H]1Oc1ccc(C#N)c(Cl)c1 ; CC1(C)[C@H](NC(=O)c2ccc(N3CCN(CC(=O)N4CCC(O)CC4)CC3)nc2)C(C)(C)[C@H]1Oc1ccc(C#N)c(Cl)c1 ; CC1(C)[C@H](NC(=O)c2cnc(OCCCO)nc2)C(C)(C)[C@H]1Oc1ccc(C#N)c(Cl)c1 ; CN(CCCO)c1ncc(C(=O)N[C@H]2C(C)(C)[C@H](Oc3ccc(C#N)c(Cl)c3)C2(C)C)cn1 ; CN(CCO)c1ncc(C(=O)N[C@H]2C(C)(C)[C@H](Oc3ccc(C#N)c(Cl)c3)C2(C)C)cn1]

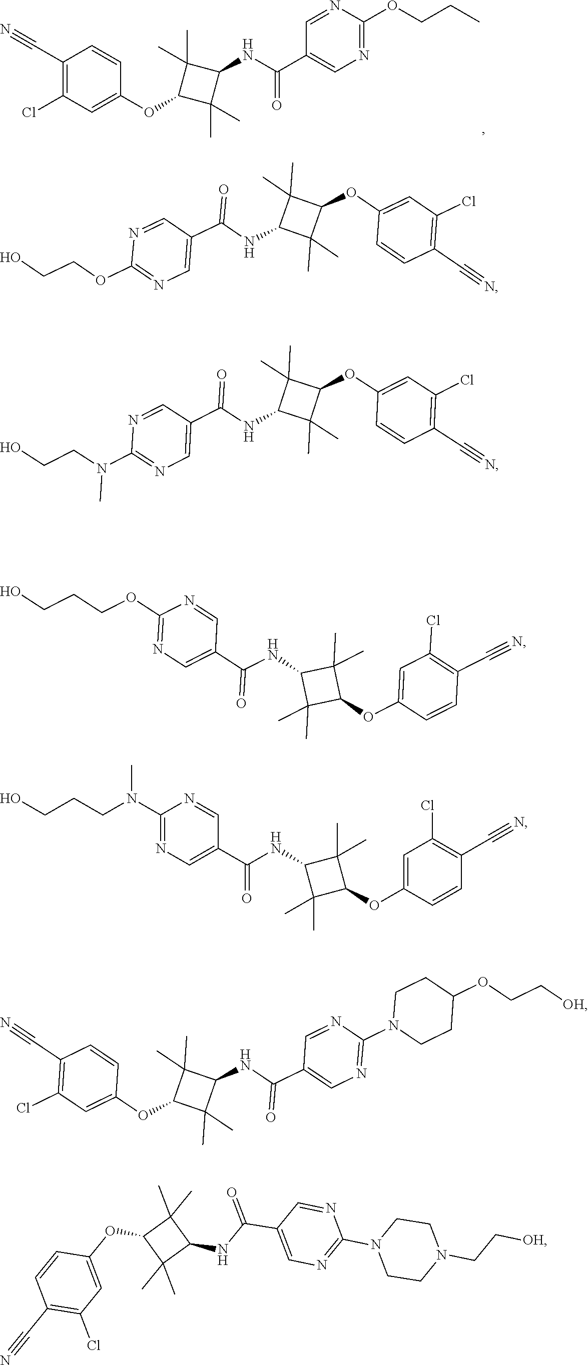

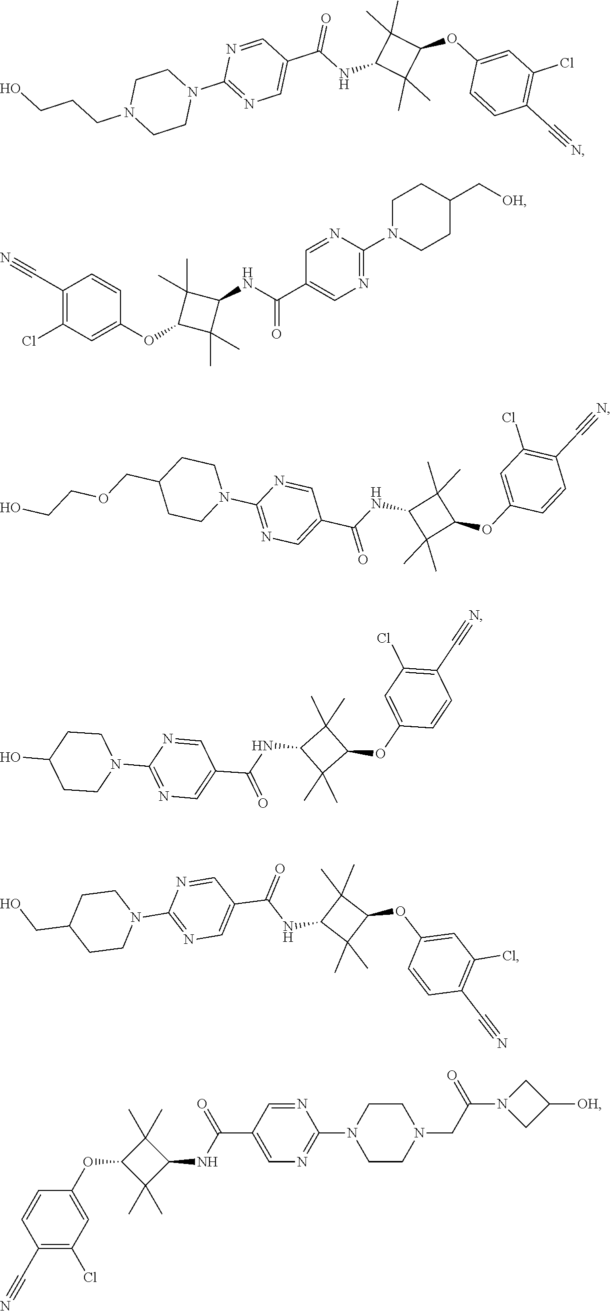

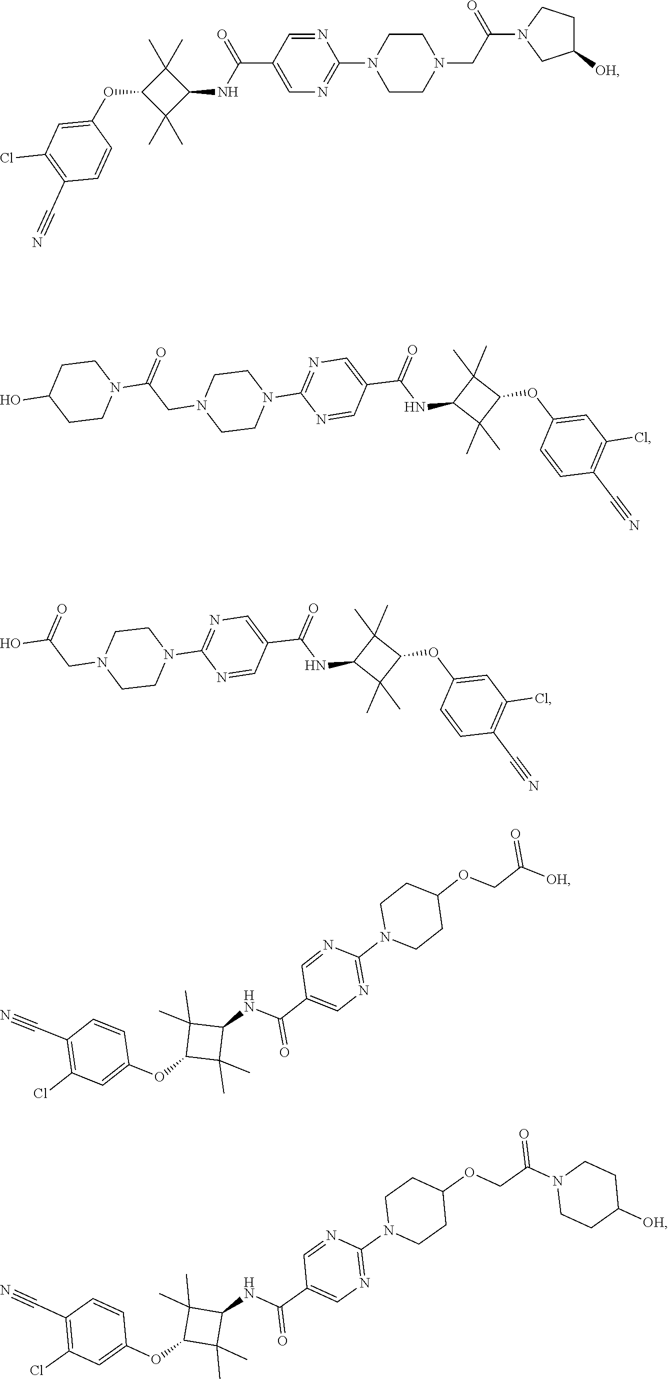 [SMILES: CC1(C)[C@H](NC(=O)c2cnc(N3CCN(CC(=O)N4CC[C@@H](O)C4)CC3)nc2)C(C)(C)[C@H]1Oc1ccc(C#N)c(Cl)c1 ; CC1(C)[C@H](NC(=O)c2cnc(N3CCN(CC(=O)N4CCC(O)CC4)CC3)nc2)C(C)(C)[C@H]1Oc1ccc(C#N)c(Cl)c1 ; CC1(C)[C@H](NC(=O)c2cnc(N3CCN(CC(=O)O)CC3)nc2)C(C)(C)[C@H]1Oc1ccc(C#N)c(Cl)c1 ; CC1(C)[C@H](NC(=O)c2cnc(N3CCC(OCC(=O)O)CC3)nc2)C(C)(C)[C@H]1Oc1ccc(C#N)c(Cl)c1 ; CC1(C)[C@H](NC(=O)c2cnc(N3CCC(OCC(=O)N4CCC(O)CC4)CC3)nc2)C(C)(C)[C@H]1Oc1ccc(C#N)c(Cl)c1]

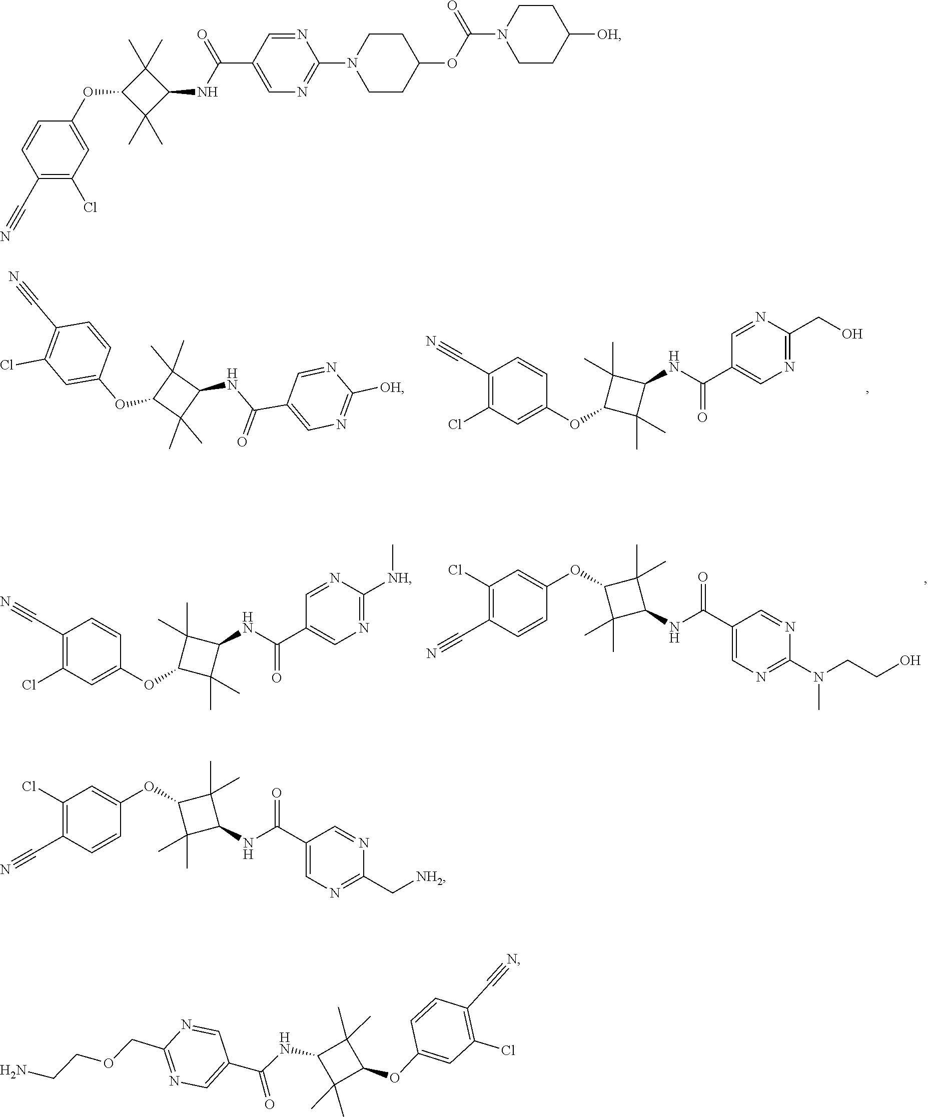

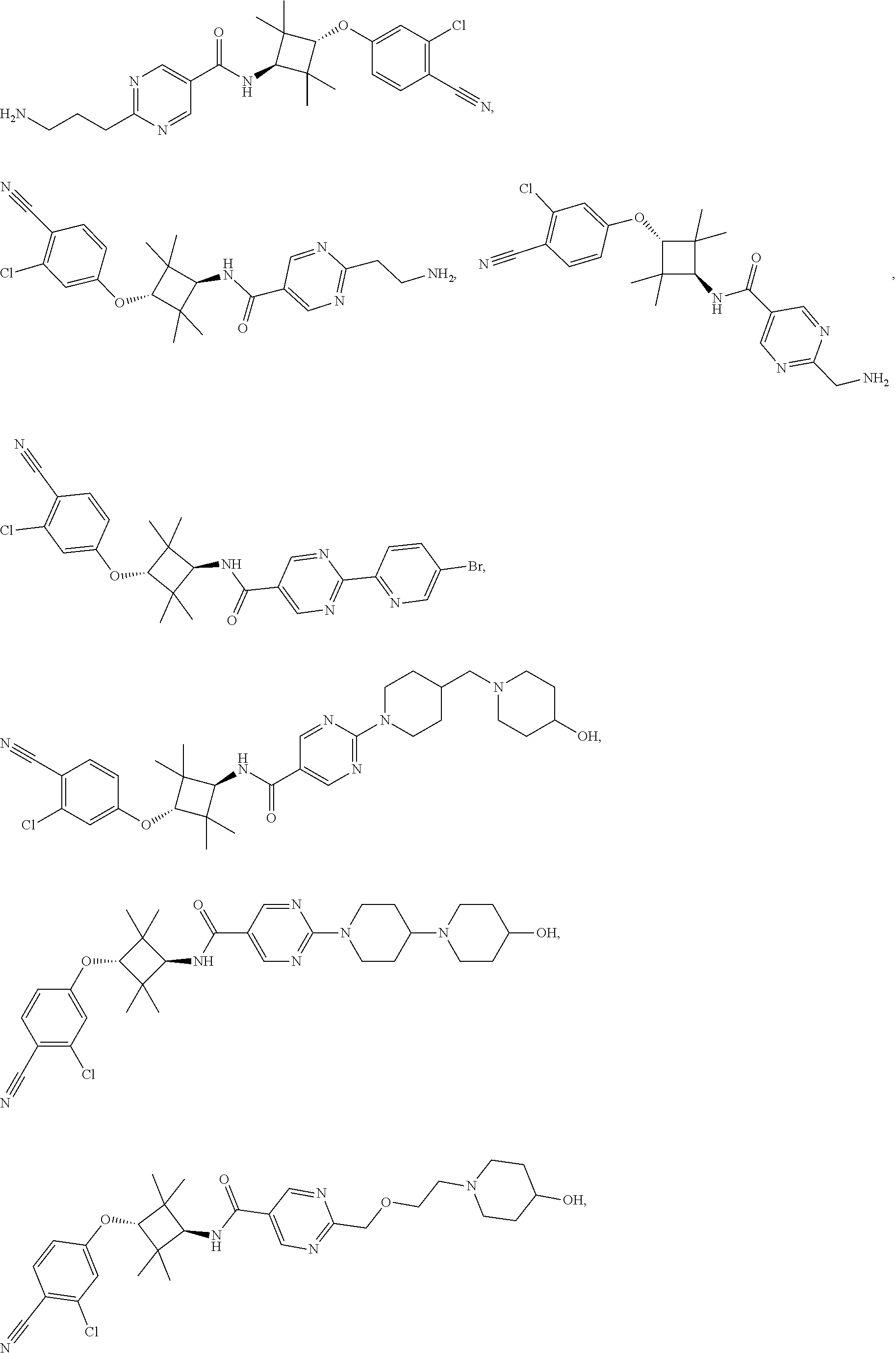

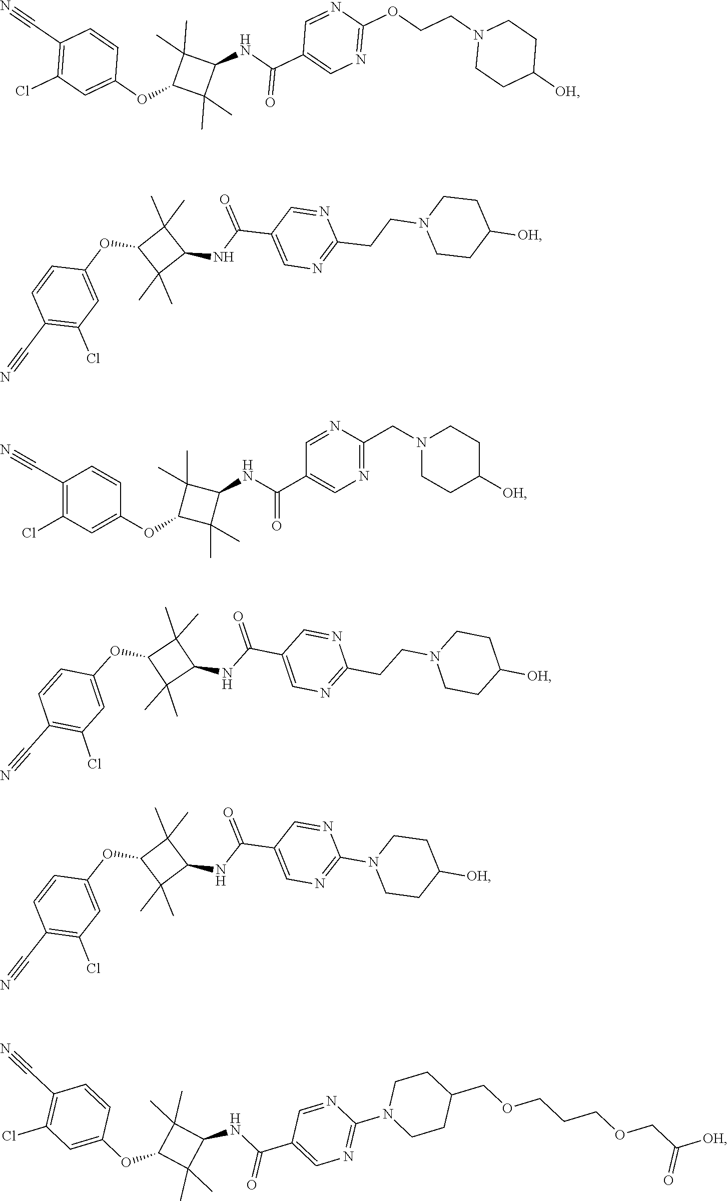 [SMILES: CC1(C)[C@H](NC(=O)c2cnc(OCCN3CCC(O)CC3)nc2)C(C)(C)[C@H]1Oc1ccc(C#N)c(Cl)c1 ; CC1(C)[C@H](NC(=O)c2cnc(CCN3CCC(O)CC3)nc2)C(C)(C)[C@H]1Oc1ccc(C#N)c(Cl)c1 ; CC1(C)[C@H](NC(=O)c2cnc(CN3CCC(O)CC3)nc2)C(C)(C)[C@H]1Oc1ccc(C#N)c(Cl)c1 ; CC1(C)[C@H](NC(=O)c2cnc(CCN3CCC(O)CC3)nc2)C(C)(C)[C@H]1Oc1ccc(C#N)c(Cl)c1 ; CC1(C)[C@H](NC(=O)c2cnc(N3CCC(O)CC3)nc2)C(C)(C)[C@H]1Oc1ccc(C#N)c(Cl)c1 ; CC1(C)[C@H](NC(=O)c2cnc(N3CCC(COCCCOCC(=O)O)CC3)nc2)C(C)(C)[C@H]1Oc1ccc(C#N)c(Cl)c1]

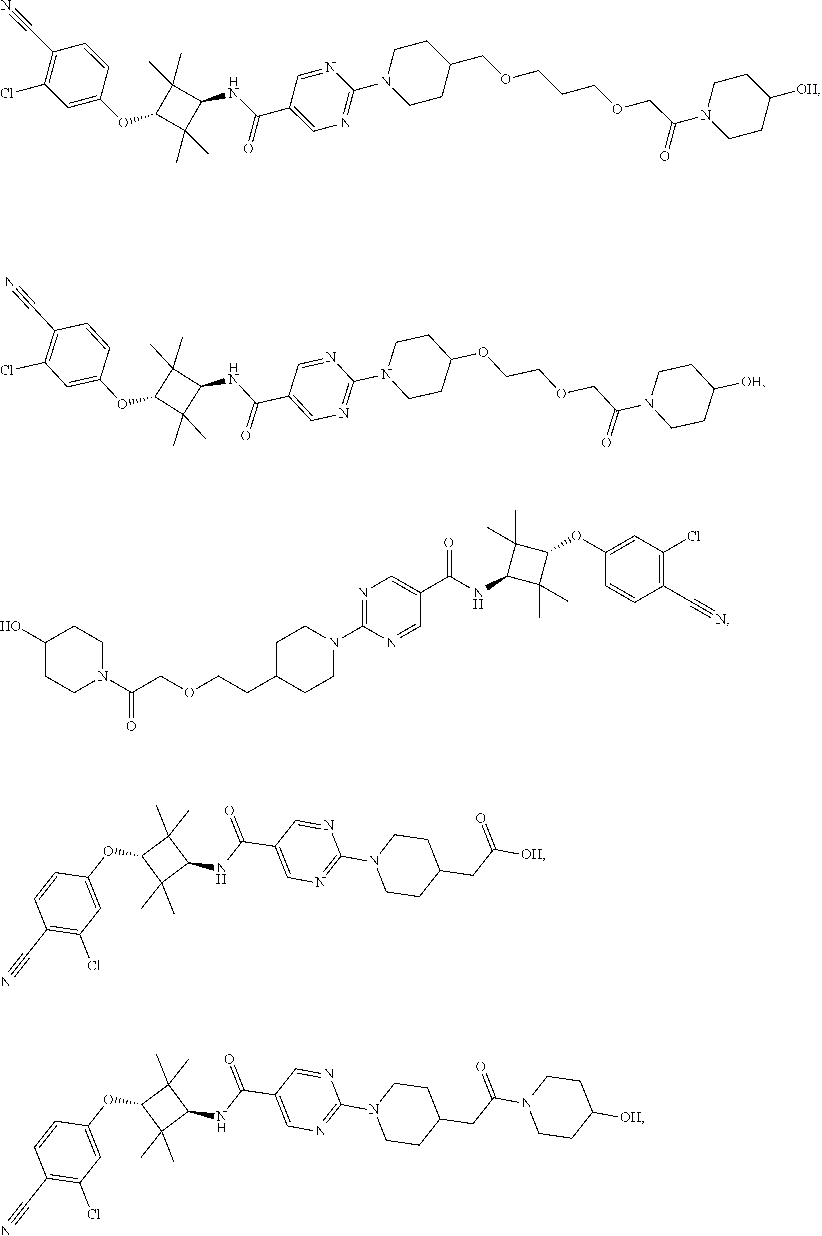 [SMILES: CC1(C)[C@H](NC(=O)c2cnc(N3CCC(COCCCOCC(=O)N4CCC(O)CC4)CC3)nc2)C(C)(C)[C@H]1Oc1ccc(C#N)c(Cl)c1 ; CC1(C)[C@H](NC(=O)c2cnc(N3CCC(OCCOCC(=O)N4CCC(O)CC4)CC3)nc2)C(C)(C)[C@H]1Oc1ccc(C#N)c(Cl)c1 ; CC1(C)[C@H](NC(=O)c2cnc(N3CCC(CCOCC(=O)N4CCC(O)CC4)CC3)nc2)C(C)(C)[C@H]1Oc1ccc(C#N)c(Cl)c1 ; CC1(C)[C@H](NC(=O)c2cnc(N3CCC(CC(=O)O)CC3)nc2)C(C)(C)[C@H]1Oc1ccc(C#N)c(Cl)c1 ; CC1(C)[C@H](NC(=O)c2cnc(N3CCC(CC(=O)N4CCC(O)CC4)CC3)nc2)C(C)(C)[C@H]1Oc1ccc(C#N)c(Cl)c1]

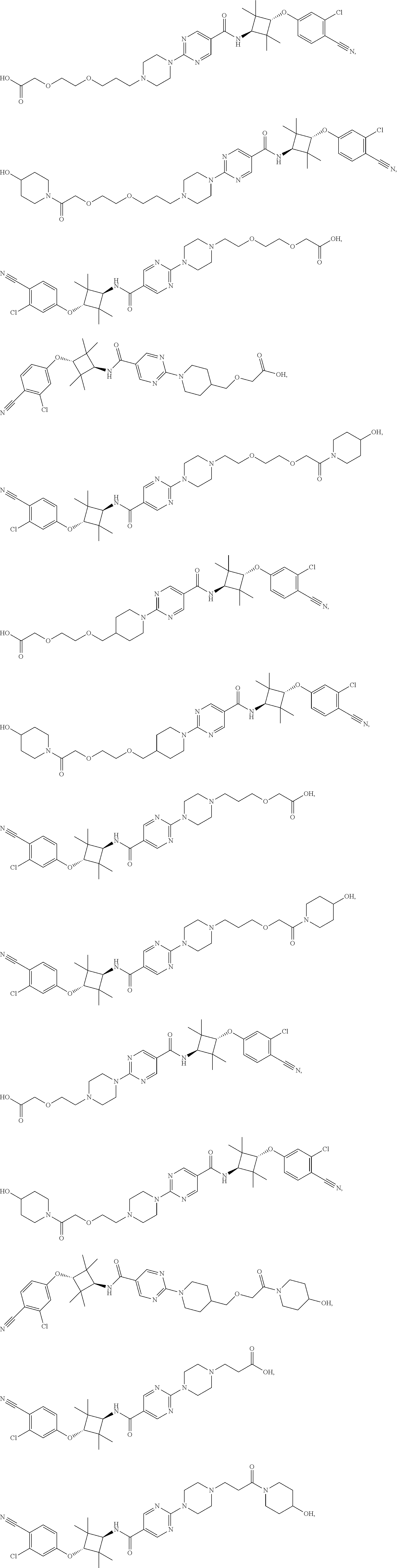

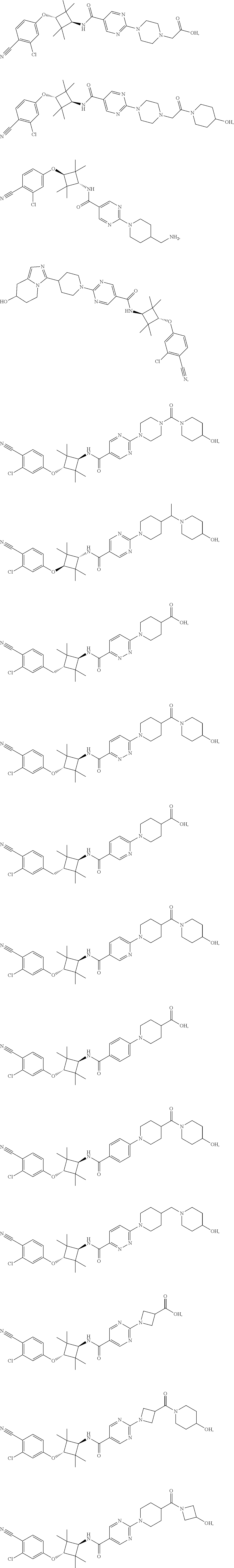

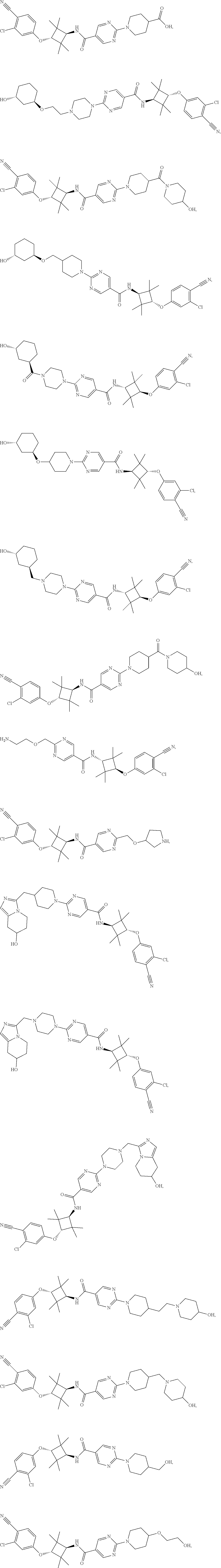

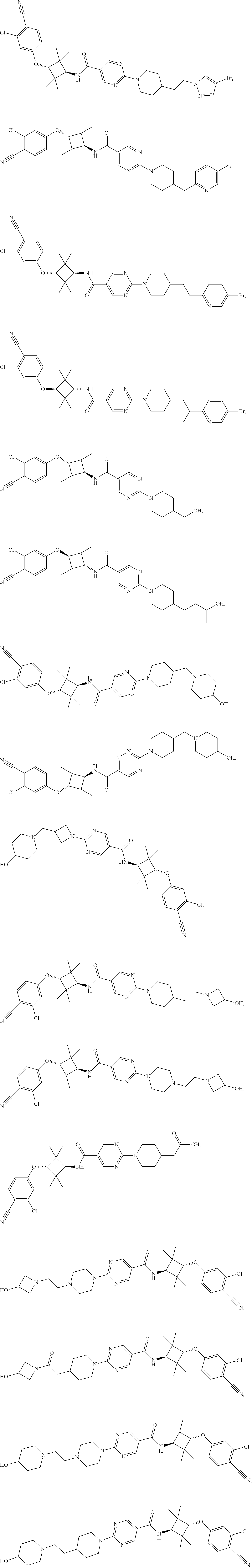

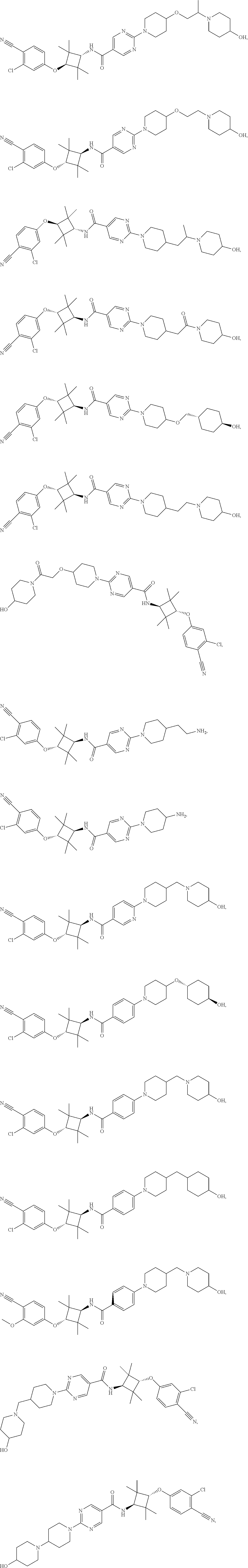

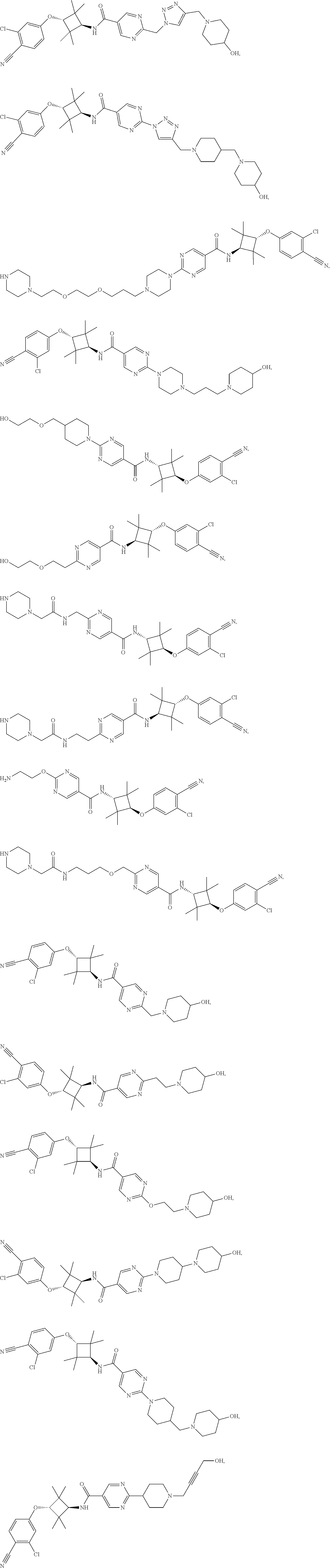

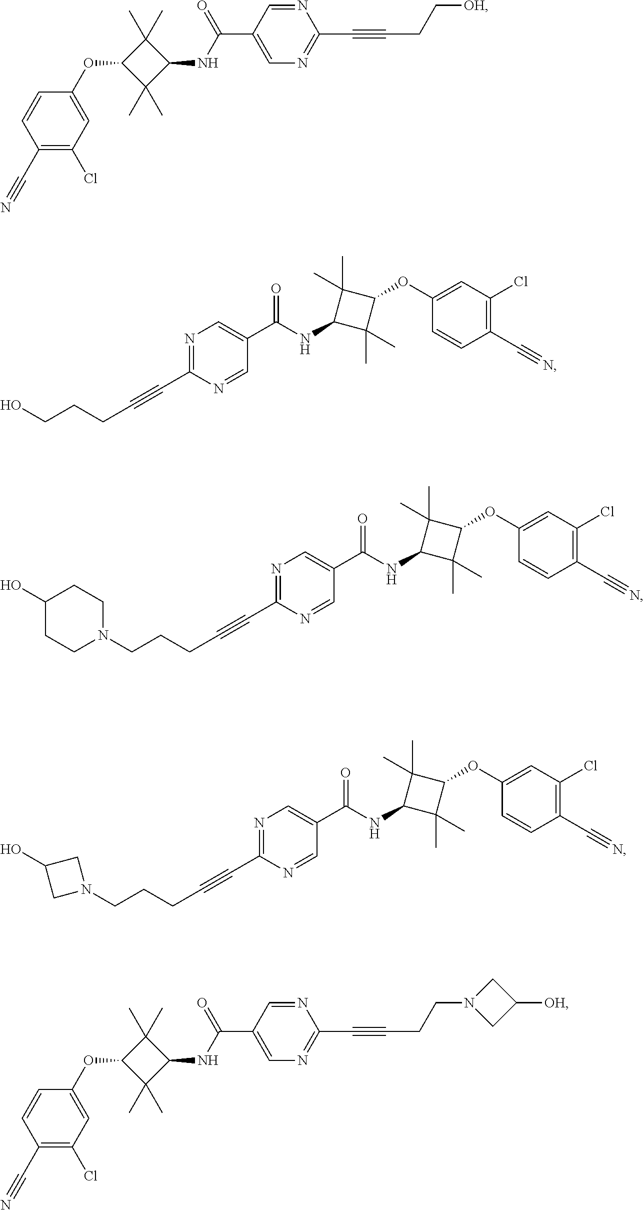 [SMILES: CC1(C)[C@H](NC(=O)c2cnc(C#CCCO)nc2)C(C)(C)[C@H]1Oc1ccc(C#N)c(Cl)c1 ; CC1(C)[C@H](NC(=O)c2cnc(C#CCCCO)nc2)C(C)(C)[C@H]1Oc1ccc(C#N)c(Cl)c1 ; CC1(C)[C@H](NC(=O)c2cnc(C#CCCCN3CCC(O)CC3)nc2)C(C)(C)[C@H]1Oc1ccc(C#N)c(Cl)c1 ; CC1(C)[C@H](NC(=O)c2cnc(C#CCCCN3CC(O)C3)nc2)C(C)(C)[C@H]1Oc1ccc(C#N)c(Cl)c1 ; CC1(C)[C@H](NC(=O)c2cnc(C#CCCN3CC(O)C3)nc2)C(C)(C)[C@H]1Oc1ccc(C#N)c(Cl)c1]

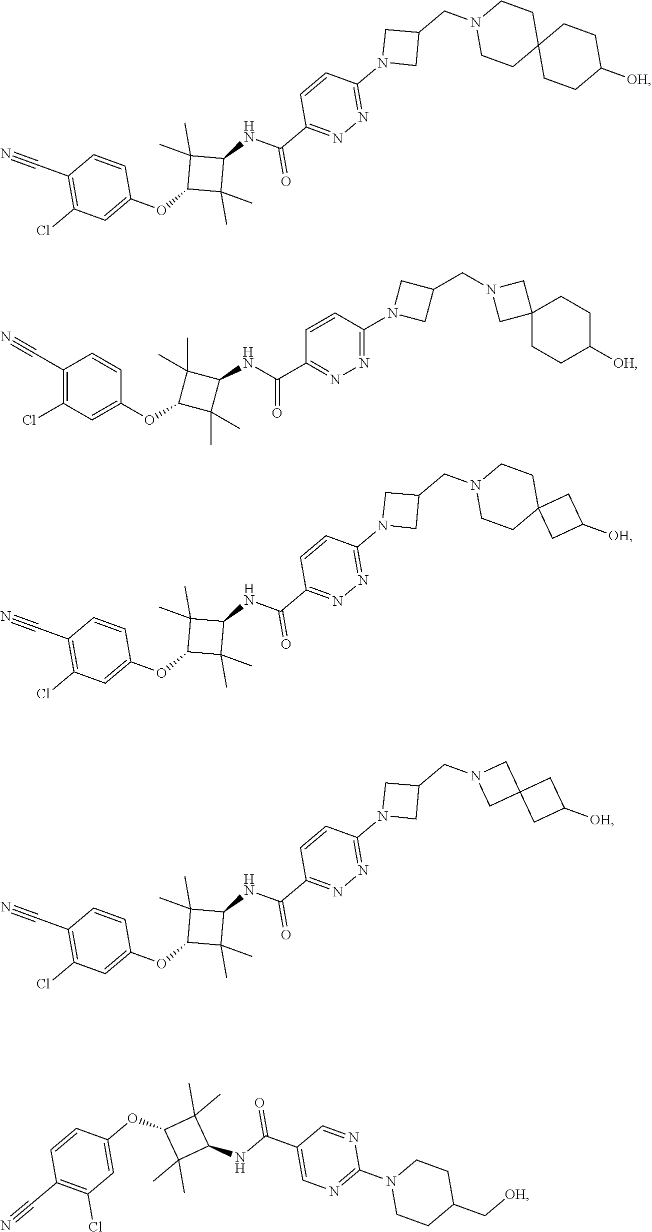 [SMILES: CC1(C)[C@H](NC(=O)c2ccc(N3CC(CN4CCC5(CCC(O)CC5)CC4)C3)nn2)C(C)(C)[C@H]1Oc1ccc(C#N)c(Cl)c1 ; CC1(C)[C@H](NC(=O)c2ccc(N3CC(CN4CC5(CCC(O)CC5)C4)C3)nn2)C(C)(C)[C@H]1Oc1ccc(C#N)c(Cl)c1 ; CC1(C)[C@H](NC(=O)c2ccc(N3CC(CN4CCC5(CC4)CC(O)C5)C3)nn2)C(C)(C)[C@H]1Oc1ccc(C#N)c(Cl)c1 ; CC1(C)[C@H](NC(=O)c2ccc(N3CC(CN4CC5(CC(O)C5)C4)C3)nn2)C(C)(C)[C@H]1Oc1ccc(C#N)c(Cl)c1 ; CC1(C)[C@H](NC(=O)c2cnc(N3CCC(CO)CC3)nc2)C(C)(C)[C@H]1Oc1ccc(C#N)c(Cl)c1]

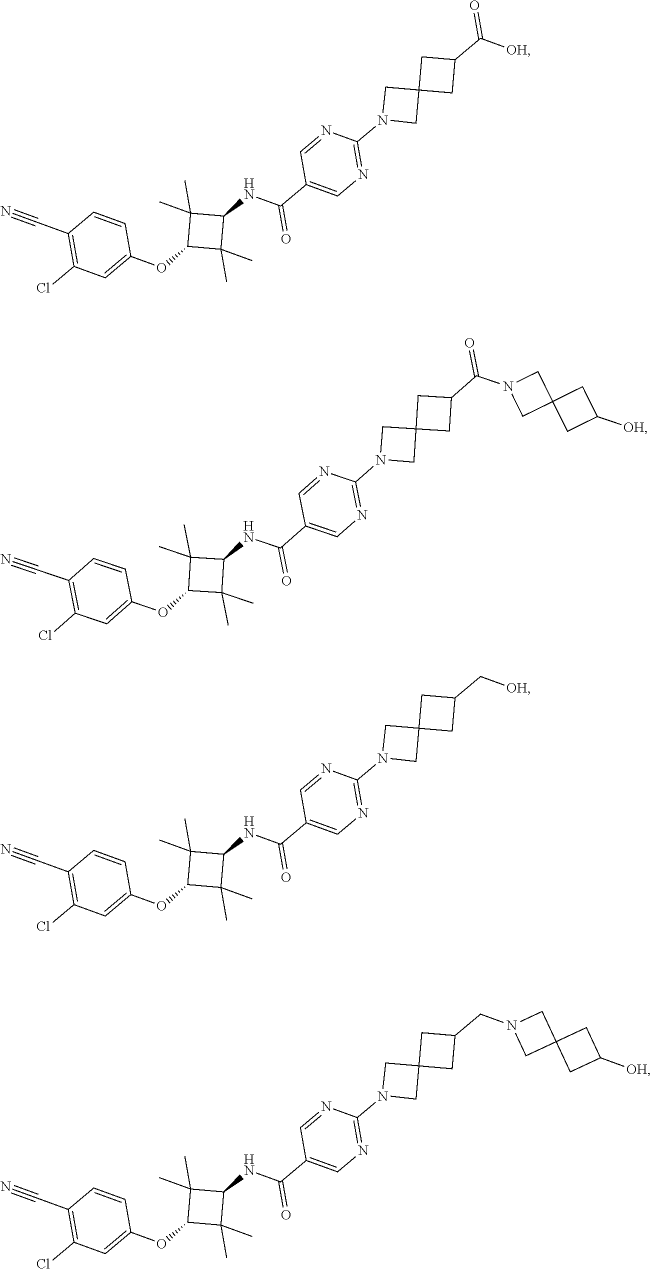 [SMILES: CC1(C)[C@H](NC(=O)c2cnc(N3CC4(CC(C(=O)O)C4)C3)nc2)C(C)(C)[C@H]1Oc1ccc(C#N)c(Cl)c1 ; CC1(C)[C@H](NC(=O)c2cnc(N3CC4(CC(C(=O)N5CC6(CC(O)C6)C5)C4)C3)nc2)C(C)(C)[C@H]1Oc1ccc(C#N)c(Cl)c1 ; CC1(C)[C@H](NC(=O)c2cnc(N3CC4(CC(CO)C4)C3)nc2)C(C)(C)[C@H]1Oc1ccc(C#N)c(Cl)c1 ; CC1(C)[C@H](NC(=O)c2cnc(N3CC4(CC(CN5CC6(CC(O)C6)C5)C4)C3)nc2)C(C)(C)[C@H]1Oc1ccc(C#N)c(Cl)c1]

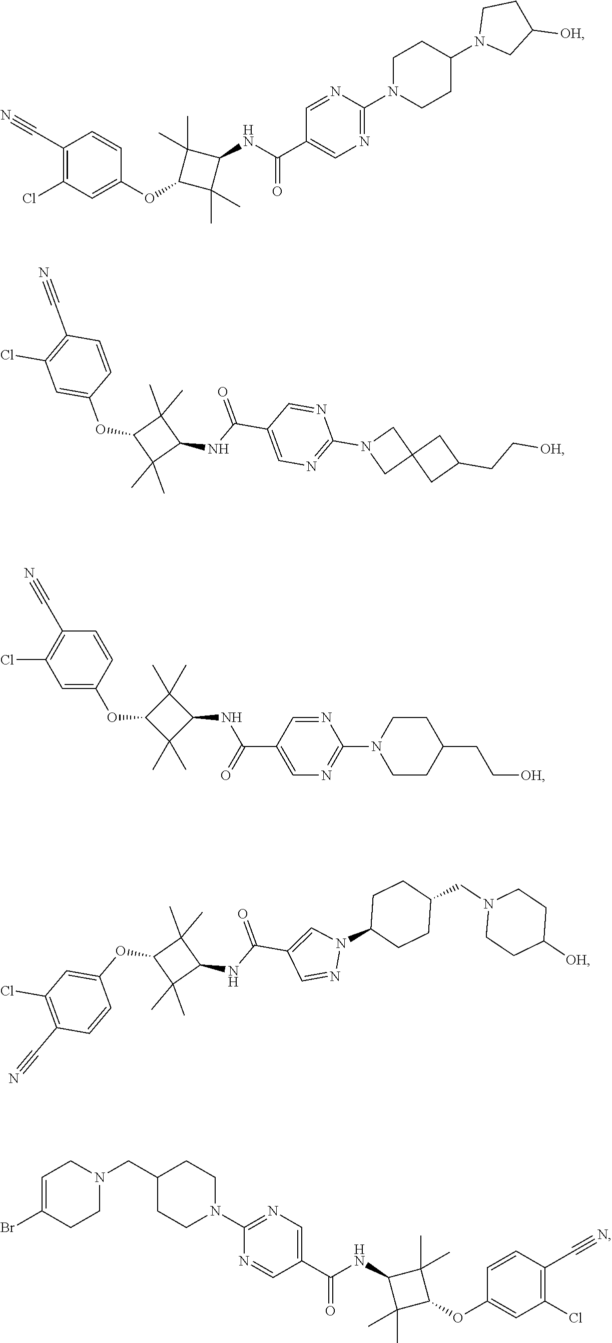 [SMILES: CC1(C)[C@H](NC(=O)c2cnc(N3CCC(N4CCC(O)C4)CC3)nc2)C(C)(C)[C@H]1Oc1ccc(C#N)c(Cl)c1 ; CC1(C)[C@H](NC(=O)c2cnc(N3CC4(CC(CCO)C4)C3)nc2)C(C)(C)[C@H]1Oc1ccc(C#N)c(Cl)c1 ; CC1(C)[C@H](NC(=O)c2cnc(N3CCC(CCO)CC3)nc2)C(C)(C)[C@H]1Oc1ccc(C#N)c(Cl)c1 ; CC1(C)[C@H](NC(=O)c2cnn([C@H]3CC[C@H](CN4CCC(O)CC4)CC3)c2)C(C)(C)[C@H]1Oc1ccc(C#N)c(Cl)c1 ; CC1(C)[C@H](NC(=O)c2cnc(N3CCC(CN4CC=C(Br)CC4)CC3)nc2)C(C)(C)[C@H]1Oc1ccc(C#N)c(Cl)c1]

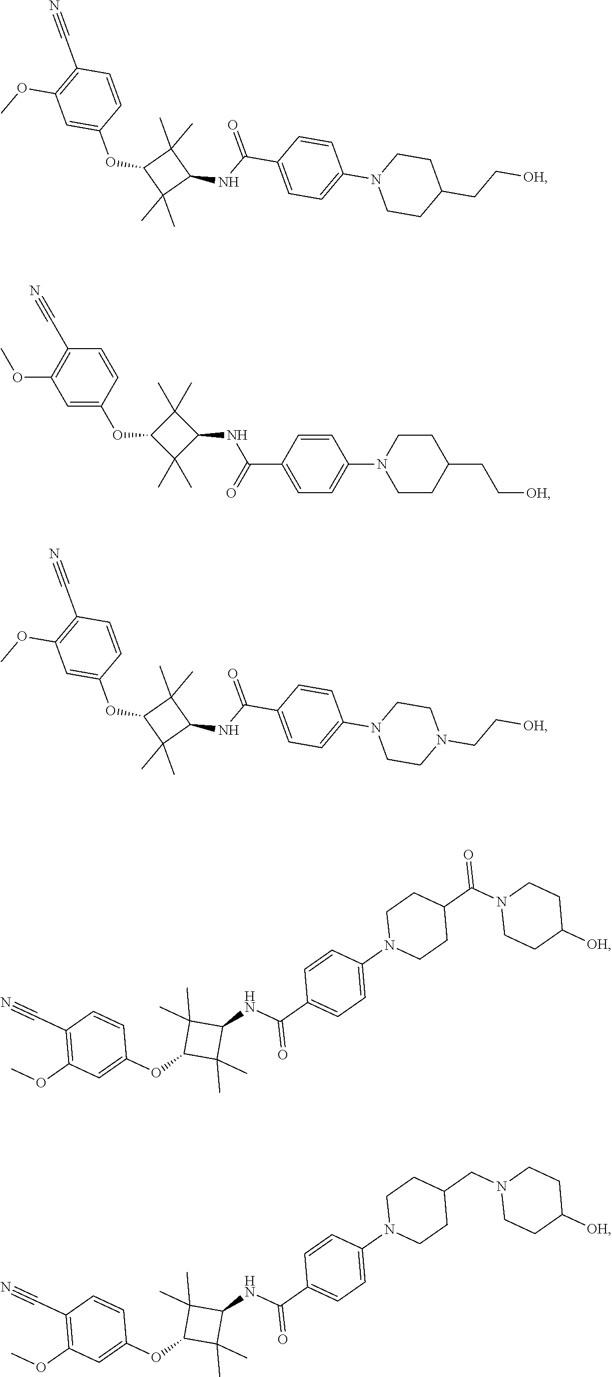 [SMILES: COc1cc(O[C@H]2C(C)(C)[C@H](NC(=O)c3ccc(N4CCC(CCO)CC4)cc3)C2(C)C)ccc1C#N ; COc1cc(O[C@H]2C(C)(C)[C@H](NC(=O)c3ccc(N4CCC(CCO)CC4)cc3)C2(C)C)ccc1C#N ; COc1cc(O[C@H]2C(C)(C)[C@H](NC(=O)c3ccc(N4CCN(CCO)CC4)cc3)C2(C)C)ccc1C#N ; COc1cc(O[C@H]2C(C)(C)[C@H](NC(=O)c3ccc(N4CCC(C(=O)N5CCC(O)CC5)CC4)cc3)C2(C)C)ccc1C#N ; COc1cc(O[C@H]2C(C)(C)[C@H](NC(=O)c3ccc(N4CCC(CN5CCC(O)CC5)CC4)cc3)C2(C)C)ccc1C#N]

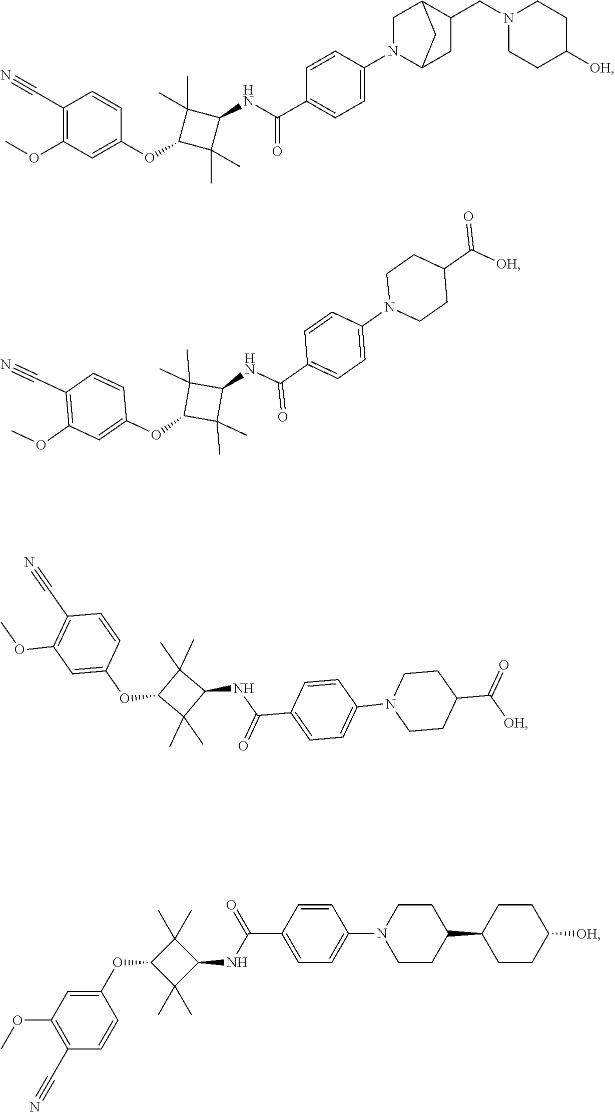 [SMILES: COc1cc(O[C@H]2C(C)(C)[C@H](NC(=O)c3ccc(N4CC5CC4CC5CN4CCC(O)CC4)cc3)C2(C)C)ccc1C#N ; COc1cc(O[C@H]2C(C)(C)[C@H](NC(=O)c3ccc(N4CCC(C(=O)O)CC4)cc3)C2(C)C)ccc1C#N ; COc1cc(O[C@H]2C(C)(C)[C@H](NC(=O)c3ccc(N4CCC(C(=O)O)CC4)cc3)C2(C)C)ccc1C#N ; COc1cc(O[C@H]2C(C)(C)[C@H](NC(=O)c3ccc(N4CCC([C@H]5CC[C@H](O)CC5)CC4)cc3)C2(C)C)ccc1C#N]

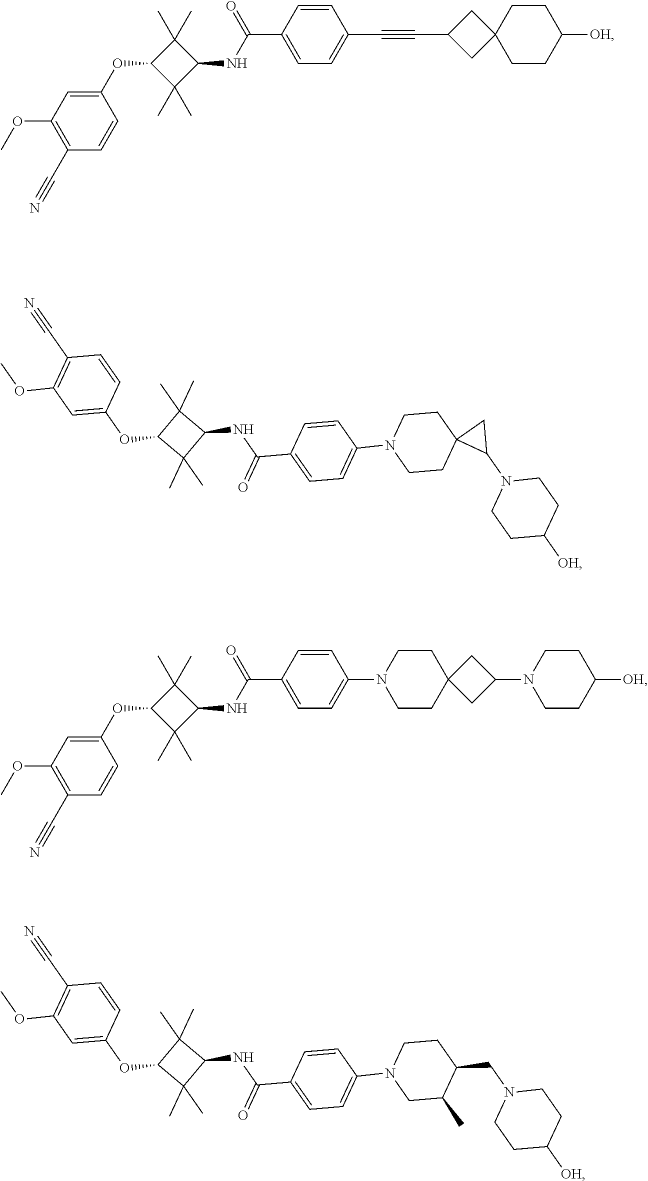 [SMILES: COc1cc(O[C@H]2C(C)(C)[C@H](NC(=O)c3ccc(C#CC4CC5(CCC(O)CC5)C4)cc3)C2(C)C)ccc1C#N ; COc1cc(O[C@H]2C(C)(C)[C@H](NC(=O)c3ccc(N4CCC5(CC4)CC5N4CCC(O)CC4)cc3)C2(C)C)ccc1C#N ; COc1cc(O[C@H]2C(C)(C)[C@H](NC(=O)c3ccc(N4CCC5(CC4)CC(N4CCC(O)CC4)C5)cc3)C2(C)C)ccc1C#N ; COc1cc(O[C@H]2C(C)(C)[C@H](NC(=O)c3ccc(N4CC[C@@H](CN5CCC(O)CC5)[C@@H](C)C4)cc3)C2(C)C)ccc1C#N]

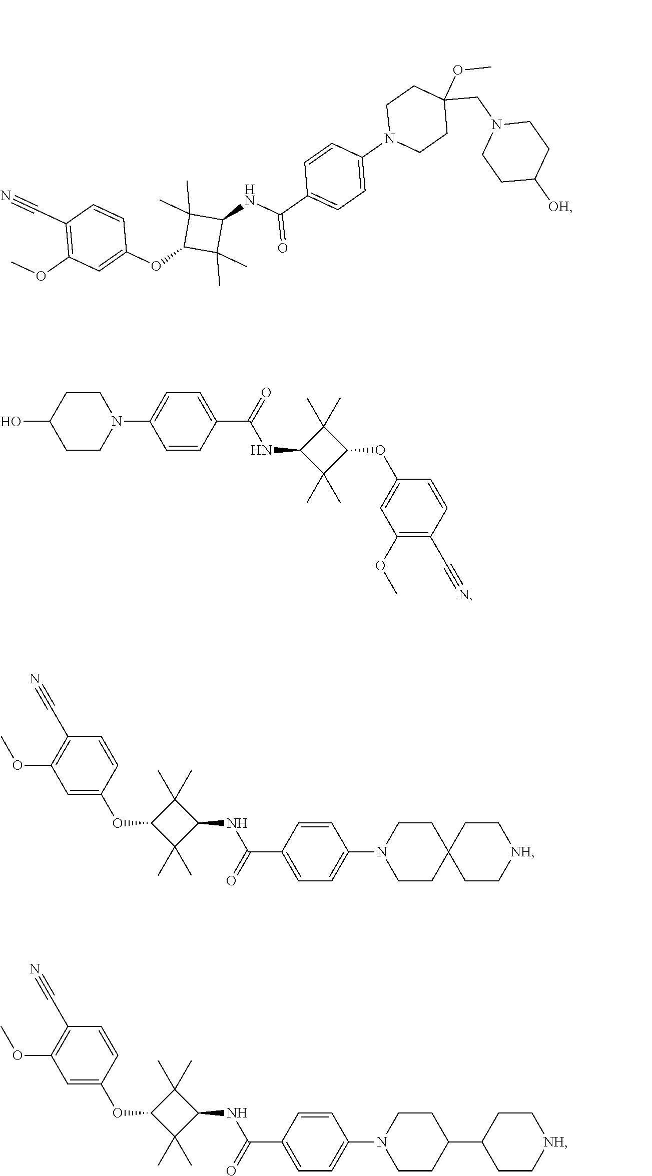 [SMILES: COc1cc(O[C@H]2C(C)(C)[C@H](NC(=O)c3ccc(N4CCC(CN5CCC(O)CC5)(OC)CC4)cc3)C2(C)C)ccc1C#N ; COc1cc(O[C@H]2C(C)(C)[C@H](NC(=O)c3ccc(N4CCC(O)CC4)cc3)C2(C)C)ccc1C#N ; COc1cc(O[C@H]2C(C)(C)[C@H](NC(=O)c3ccc(N4CCC5(CCNCC5)CC4)cc3)C2(C)C)ccc1C#N ; COc1cc(O[C@H]2C(C)(C)[C@H](NC(=O)c3ccc(N4CCC(C5CCNCC5)CC4)cc3)C2(C)C)ccc1C#N]

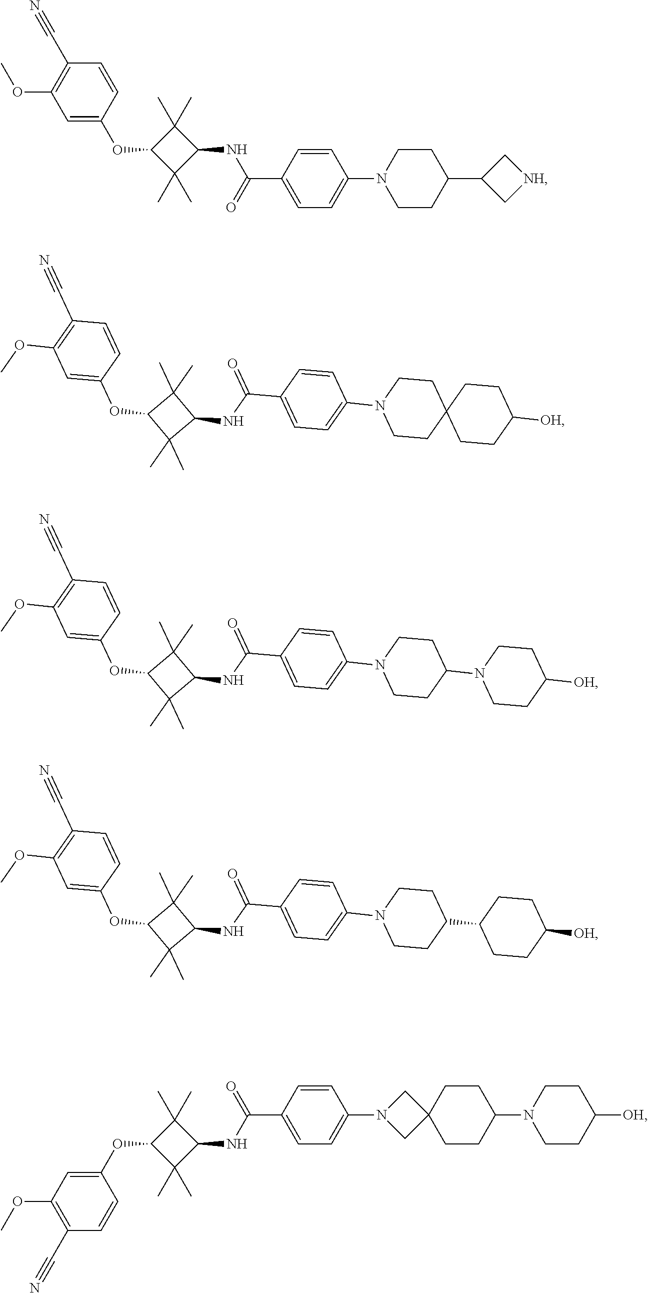 [SMILES: COc1cc(O[C@H]2C(C)(C)[C@H](NC(=O)c3ccc(N4CCC(C5CNC5)CC4)cc3)C2(C)C)ccc1C#N ; COc1cc(O[C@H]2C(C)(C)[C@H](NC(=O)c3ccc(N4CCC5(CCC(O)CC5)CC4)cc3)C2(C)C)ccc1C#N ; COc1cc(O[C@H]2C(C)(C)[C@H](NC(=O)c3ccc(N4CCC(N5CCC(O)CC5)CC4)cc3)C2(C)C)ccc1C#N ; COc1cc(O[C@H]2C(C)(C)[C@H](NC(=O)c3ccc(N4CCC([C@H]5CC[C@H](O)CC5)CC4)cc3)C2(C)C)ccc1C#N ; COc1cc(O[C@H]2C(C)(C)[C@H](NC(=O)c3ccc(N4CC5(CCC(N6CCC(O)CC6)CC5)C4)cc3)C2(C)C)ccc1C#N]

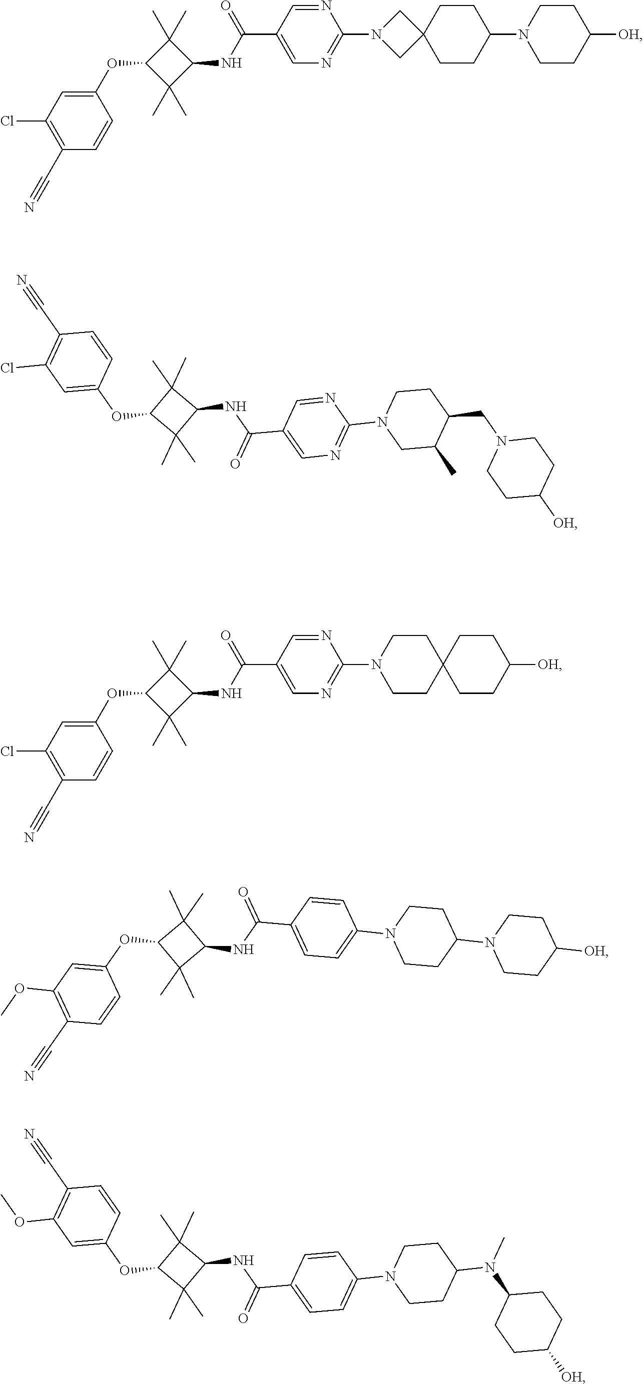 [SMILES: CC1(C)[C@H](NC(=O)c2cnc(N3CC4(CCC(N5CCC(O)CC5)CC4)C3)nc2)C(C)(C)[C@H]1Oc1ccc(C#N)c(Cl)c1 ; C[C@H]1CN(c2ncc(C(=O)N[C@H]3C(C)(C)[C@H](Oc4ccc(C#N)c(Cl)c4)C3(C)C)cn2)CC[C@H]1CN1CCC(O)CC1 ; CC1(C)[C@H](NC(=O)c2cnc(N3CCC4(CCC(O)CC4)CC3)nc2)C(C)(C)[C@H]1Oc1ccc(C#N)c(Cl)c1 ; COc1cc(O[C@H]2C(C)(C)[C@H](NC(=O)c3ccc(N4CCC(N5CCC(O)CC5)CC4)cc3)C2(C)C)ccc1C#N ; COc1cc(O[C@H]2C(C)(C)[C@H](NC(=O)c3ccc(N4CCC(N(C)[C@H]5CC[C@H](O)CC5)CC4)cc3)C2(C)C)ccc1C#N]

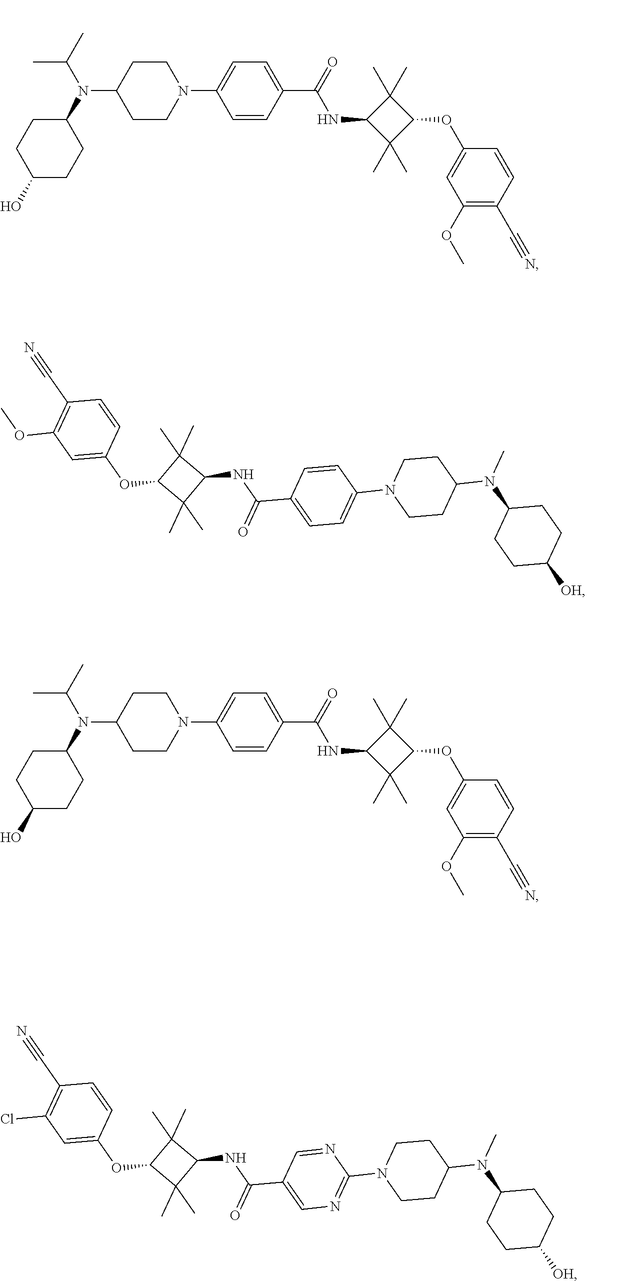 [SMILES: COc1cc(O[C@H]2C(C)(C)[C@H](NC(=O)c3ccc(N4CCC(N(C(C)C)[C@H]5CC[C@H](O)CC5)CC4)cc3)C2(C)C)ccc1C#N ; COc1cc(O[C@H]2C(C)(C)[C@H](NC(=O)c3ccc(N4CCC(N(C)[C@H]5CC[C@@H](O)CC5)CC4)cc3)C2(C)C)ccc1C#N ; COc1cc(O[C@H]2C(C)(C)[C@H](NC(=O)c3ccc(N4CCC(N(C(C)C)[C@H]5CC[C@@H](O)CC5)CC4)cc3)C2(C)C)ccc1C#N ; CN(C1CCN(c2ncc(C(=O)N[C@H]3C(C)(C)[C@H](Oc4ccc(C#N)c(Cl)c4)C3(C)C)cn2)CC1)[C@H]1CC[C@H](O)CC1]

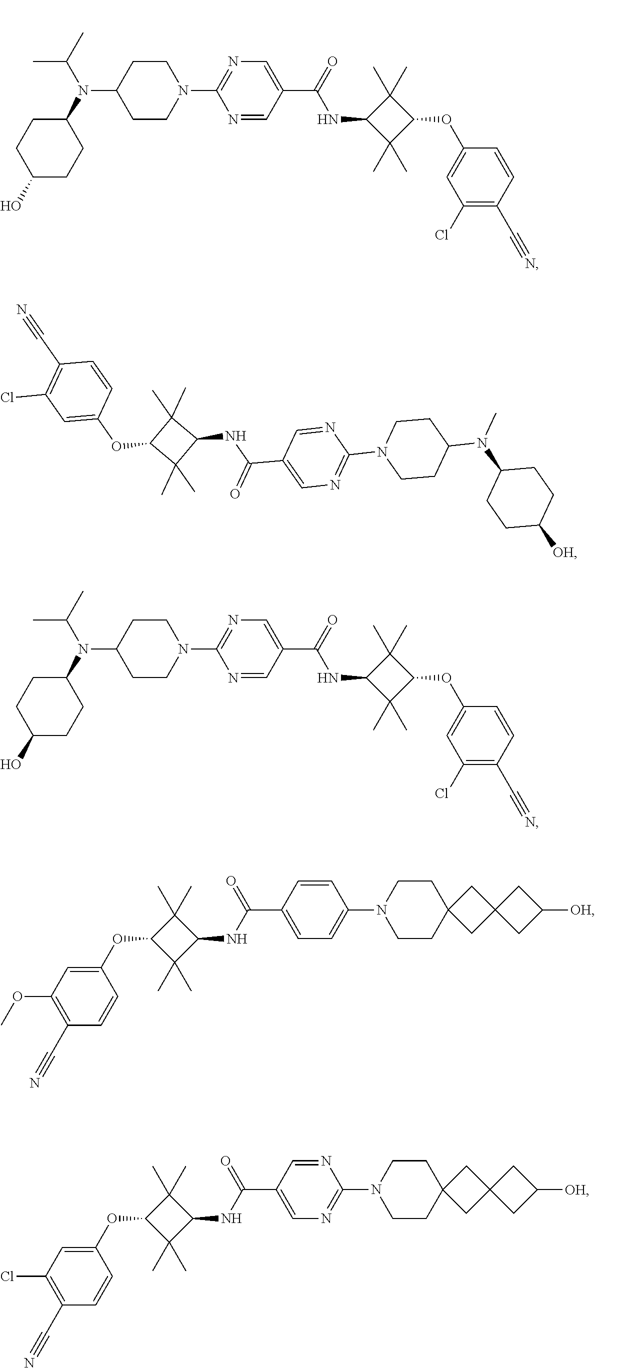 [SMILES: CC(C)N(C1CCN(c2ncc(C(=O)N[C@H]3C(C)(C)[C@H](Oc4ccc(C#N)c(Cl)c4)C3(C)C)cn2)CC1)[C@H]1CC[C@H](O)CC1 ; CN(C1CCN(c2ncc(C(=O)N[C@H]3C(C)(C)[C@H](Oc4ccc(C#N)c(Cl)c4)C3(C)C)cn2)CC1)[C@H]1CC[C@@H](O)CC1 ; CC(C)N(C1CCN(c2ncc(C(=O)N[C@H]3C(C)(C)[C@H](Oc4ccc(C#N)c(Cl)c4)C3(C)C)cn2)CC1)[C@H]1CC[C@@H](O)CC1 ; COc1cc(O[C@H]2C(C)(C)[C@H](NC(=O)c3ccc(N4CCC5(CC4)CC4(CC(O)C4)C5)cc3)C2(C)C)ccc1C#N ; CC1(C)[C@H](NC(=O)c2cnc(N3CCC4(CC3)CC3(CC(O)C3)C4)nc2)C(C)(C)[C@H]1Oc1ccc(C#N)c(Cl)c1]

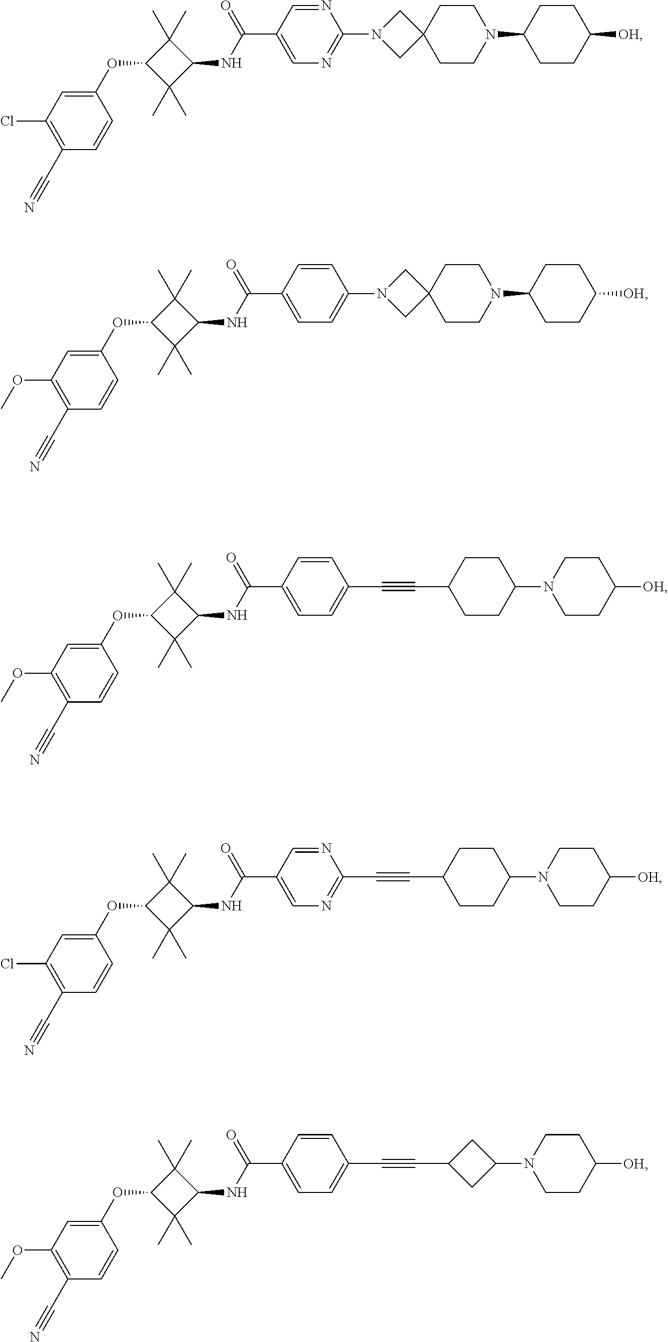 [SMILES: CC1(C)[C@H](NC(=O)c2cnc(N3CC4(CCN([C@H]5CC[C@@H](O)CC5)CC4)C3)nc2)C(C)(C)[C@H]1Oc1ccc(C#N)c(Cl)c1 ; COc1cc(O[C@H]2C(C)(C)[C@H](NC(=O)c3ccc(N4CC5(CCN([C@H]6CC[C@H](O)CC6)CC5)C4)cc3)C2(C)C)ccc1C#N ; COc1cc(O[C@H]2C(C)(C)[C@H](NC(=O)c3ccc(C#CC4CCC(N5CCC(O)CC5)CC4)cc3)C2(C)C)ccc1C#N ; CC1(C)[C@H](NC(=O)c2cnc(C#CC3CCC(N4CCC(O)CC4)CC3)nc2)C(C)(C)[C@H]1Oc1ccc(C#N)c(Cl)c1 ; COc1cc(O[C@H]2C(C)(C)[C@H](NC(=O)c3ccc(C#CC4CC(N5CCC(O)CC5)C4)cc3)C2(C)C)ccc1C#N]

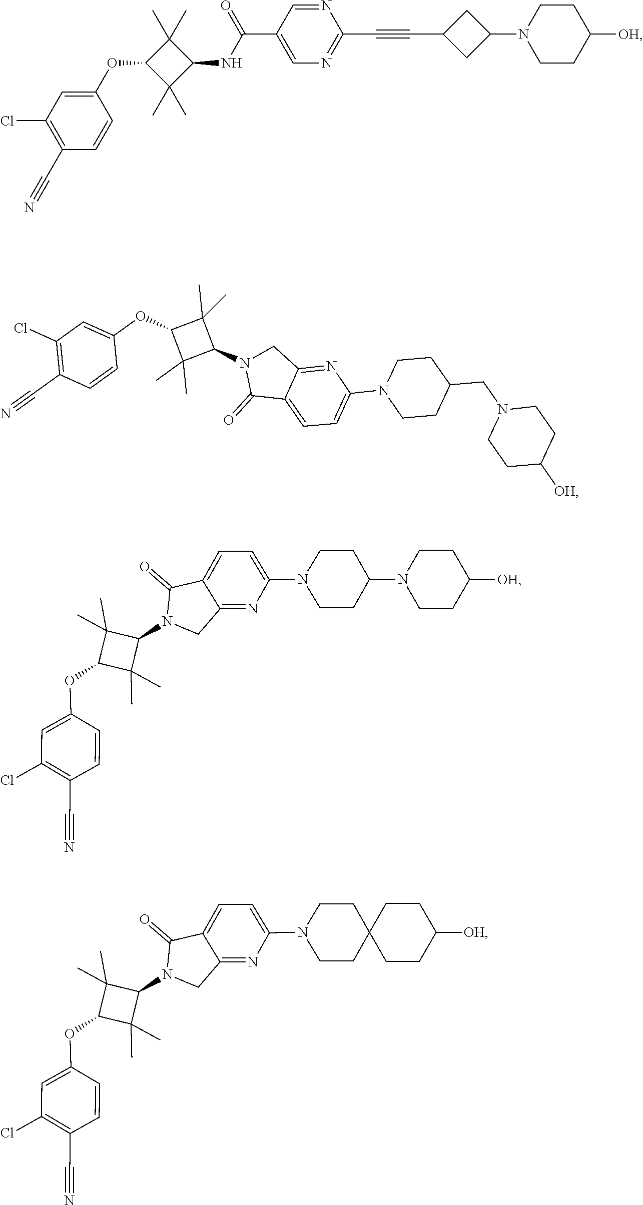 [SMILES: CC1(C)[C@H](NC(=O)c2cnc(C#CC3CC(N4CCC(O)CC4)C3)nc2)C(C)(C)[C@H]1Oc1ccc(C#N)c(Cl)c1 ; CC1(C)[C@H](Oc2ccc(C#N)c(Cl)c2)C(C)(C)[C@H]1N1Cc2nc(N3CCC(CN4CCC(O)CC4)CC3)ccc2C1=O ; CC1(C)[C@H](Oc2ccc(C#N)c(Cl)c2)C(C)(C)[C@H]1N1Cc2nc(N3CCC(N4CCC(O)CC4)CC3)ccc2C1=O ; CC1(C)[C@H](Oc2ccc(C#N)c(Cl)c2)C(C)(C)[C@H]1N1Cc2nc(N3CCC4(CCC(O)CC4)CC3)ccc2C1=O]

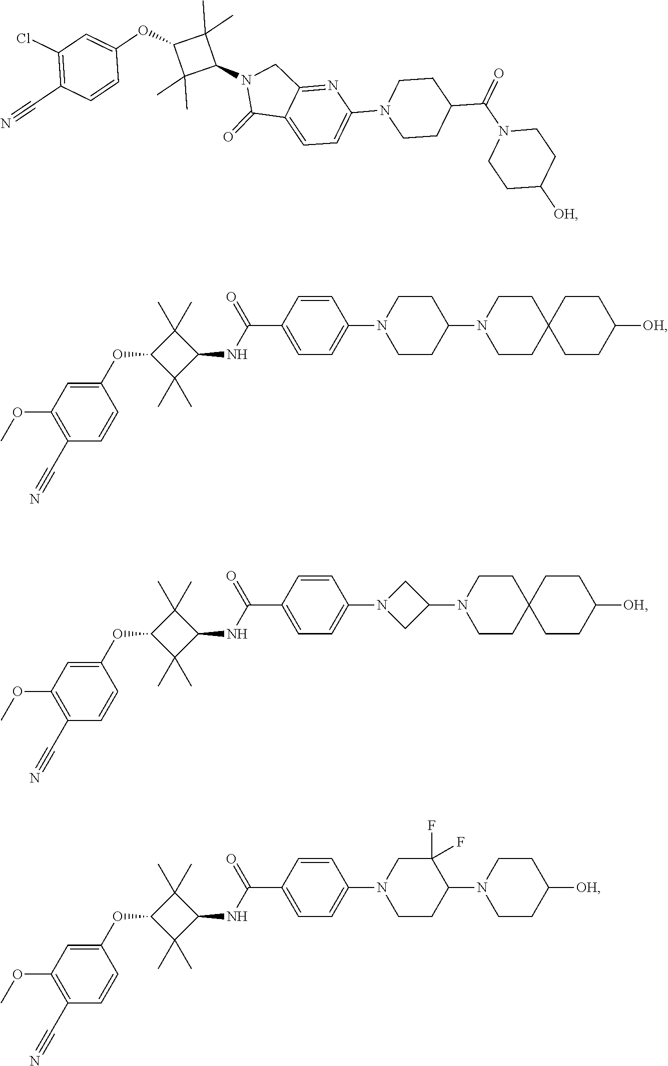 [SMILES: CC1(C)[C@H](Oc2ccc(C#N)c(Cl)c2)C(C)(C)[C@H]1N1Cc2nc(N3CCC(C(=O)N4CCC(O)CC4)CC3)ccc2C1=O ; COc1cc(O[C@H]2C(C)(C)[C@H](NC(=O)c3ccc(N4CCC(N5CCC6(CCC(O)CC6)CC5)CC4)cc3)C2(C)C)ccc1C#N ; COc1cc(O[C@H]2C(C)(C)[C@H](NC(=O)c3ccc(N4CC(N5CCC6(CCC(O)CC6)CC5)C4)cc3)C2(C)C)ccc1C#N ; COc1cc(O[C@H]2C(C)(C)[C@H](NC(=O)c3ccc(N4CCC(N5CCC(O)CC5)C(F)(F)C4)cc3)C2(C)C)ccc1C#N]

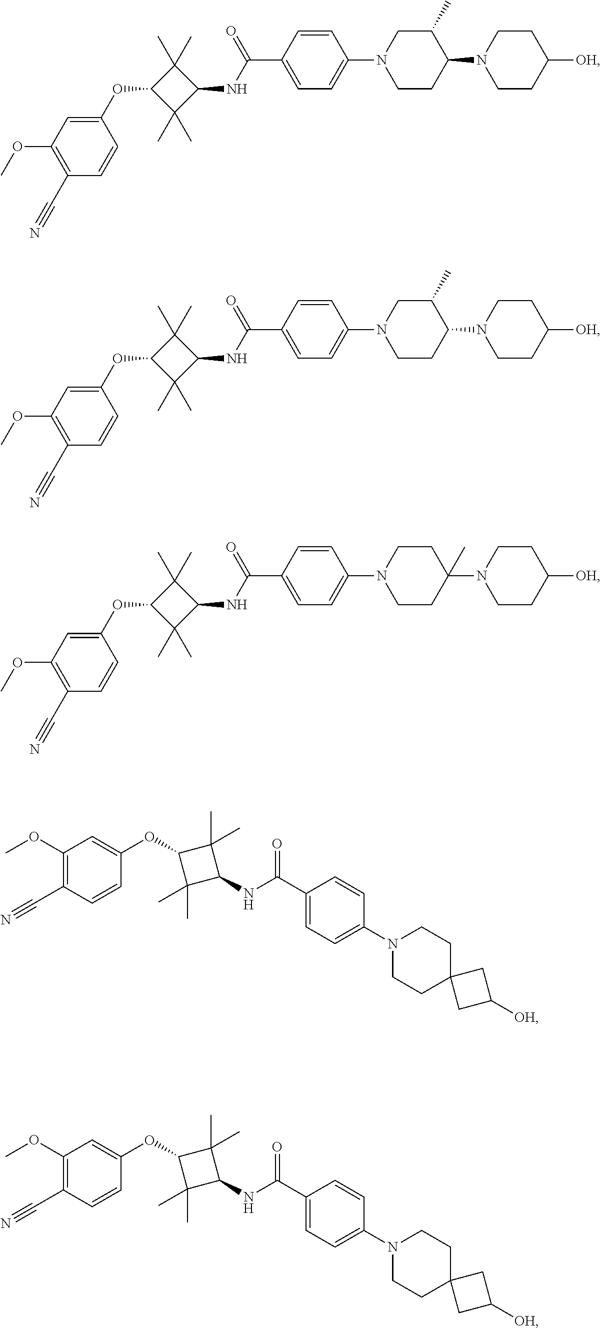 [SMILES: COc1cc(O[C@H]2C(C)(C)[C@H](NC(=O)c3ccc(N4CC[C@H](N5CCC(O)CC5)[C@@H](C)C4)cc3)C2(C)C)ccc1C#N ; COc1cc(O[C@H]2C(C)(C)[C@H](NC(=O)c3ccc(N4CC[C@@H](N5CCC(O)CC5)[C@@H](C)C4)cc3)C2(C)C)ccc1C#N ; COc1cc(O[C@H]2C(C)(C)[C@H](NC(=O)c3ccc(N4CCC(C)(N5CCC(O)CC5)CC4)cc3)C2(C)C)ccc1C#N ; COc1cc(O[C@H]2C(C)(C)[C@H](NC(=O)c3ccc(N4CCC5(CC4)CC(O)C5)cc3)C2(C)C)ccc1C#N ; COc1cc(O[C@H]2C(C)(C)[C@H](NC(=O)c3ccc(N4CCC5(CC4)CC(O)C5)cc3)C2(C)C)ccc1C#N]

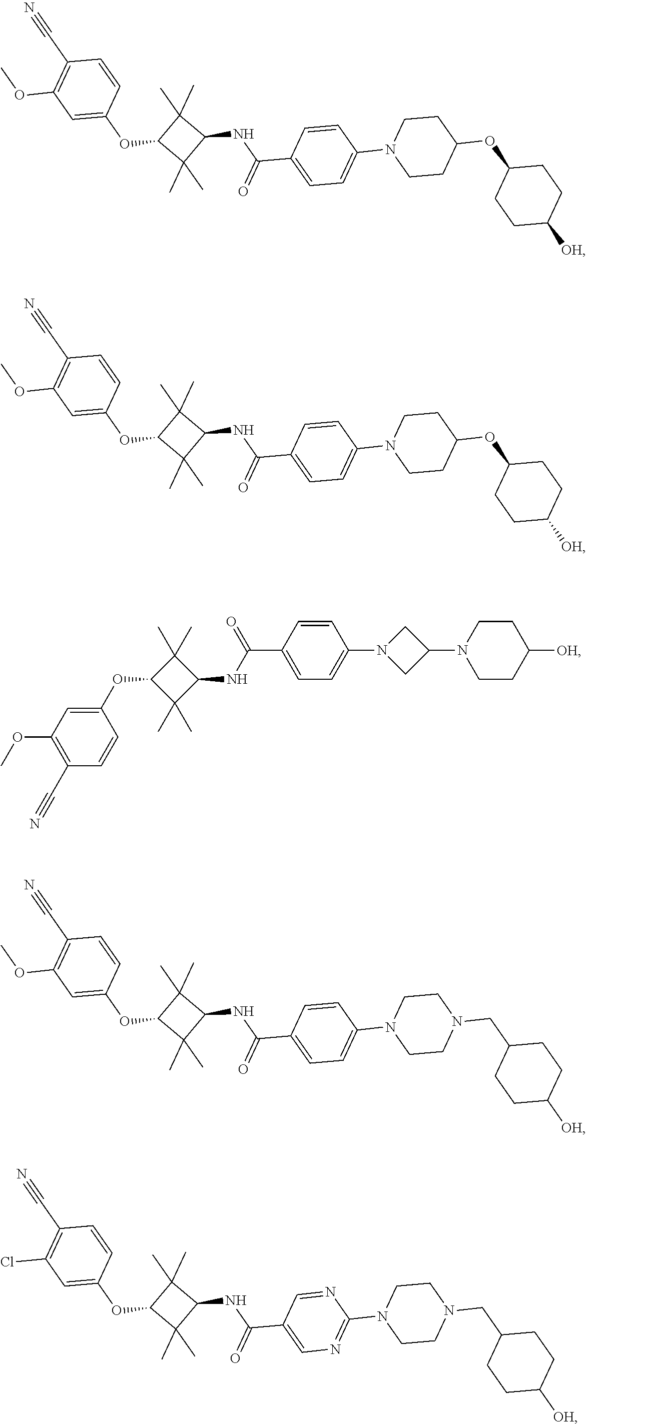 [SMILES: COc1cc(O[C@H]2C(C)(C)[C@H](NC(=O)c3ccc(N4CCC(O[C@H]5CC[C@@H](O)CC5)CC4)cc3)C2(C)C)ccc1C#N ; COc1cc(O[C@H]2C(C)(C)[C@H](NC(=O)c3ccc(N4CCC(O[C@H]5CC[C@H](O)CC5)CC4)cc3)C2(C)C)ccc1C#N ; COc1cc(O[C@H]2C(C)(C)[C@H](NC(=O)c3ccc(N4CC(N5CCC(O)CC5)C4)cc3)C2(C)C)ccc1C#N ; COc1cc(O[C@H]2C(C)(C)[C@H](NC(=O)c3ccc(N4CCN(CC5CCC(O)CC5)CC4)cc3)C2(C)C)ccc1C#N ; CC1(C)[C@H](NC(=O)c2cnc(N3CCN(CC4CCC(O)CC4)CC3)nc2)C(C)(C)[C@H]1Oc1ccc(C#N)c(Cl)c1]

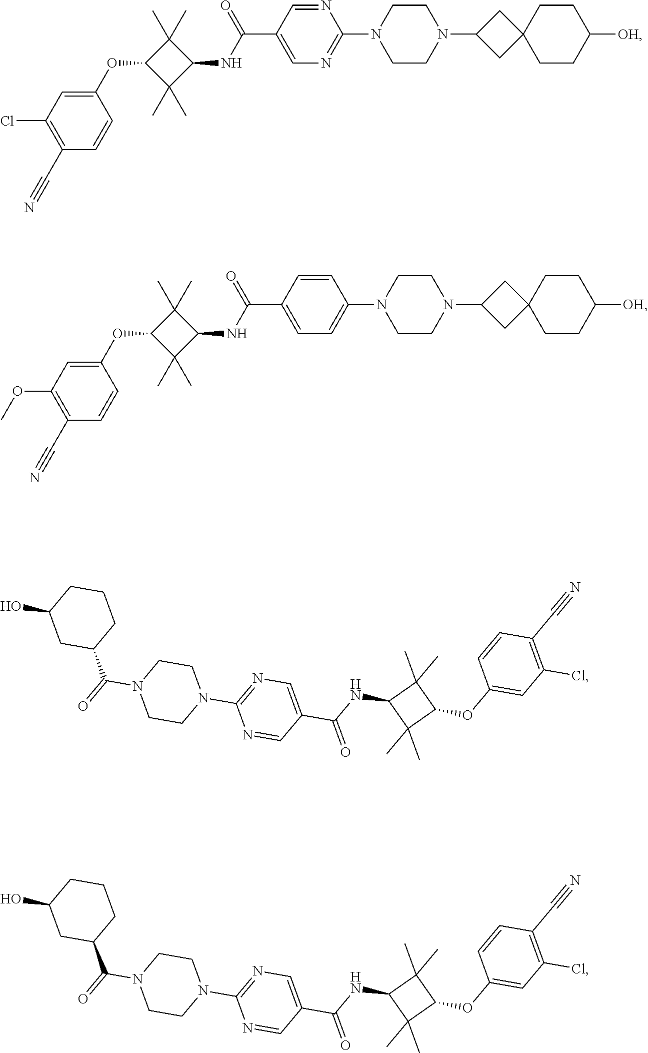 [SMILES: CC1(C)[C@H](NC(=O)c2cnc(N3CCN(C4CC5(CCC(O)CC5)C4)CC3)nc2)C(C)(C)[C@H]1Oc1ccc(C#N)c(Cl)c1 ; COc1cc(O[C@H]2C(C)(C)[C@H](NC(=O)c3ccc(N4CCN(C5CC6(CCC(O)CC6)C5)CC4)cc3)C2(C)C)ccc1C#N ; CC1(C)[C@H](NC(=O)c2cnc(N3CCN(C(=O)[C@H]4CCC[C@H](O)C4)CC3)nc2)C(C)(C)[C@H]1Oc1ccc(C#N)c(Cl)c1 ; CC1(C)[C@H](NC(=O)c2cnc(N3CCN(C(=O)[C@@H]4CCC[C@H](O)C4)CC3)nc2)C(C)(C)[C@H]1Oc1ccc(C#N)c(Cl)c1]

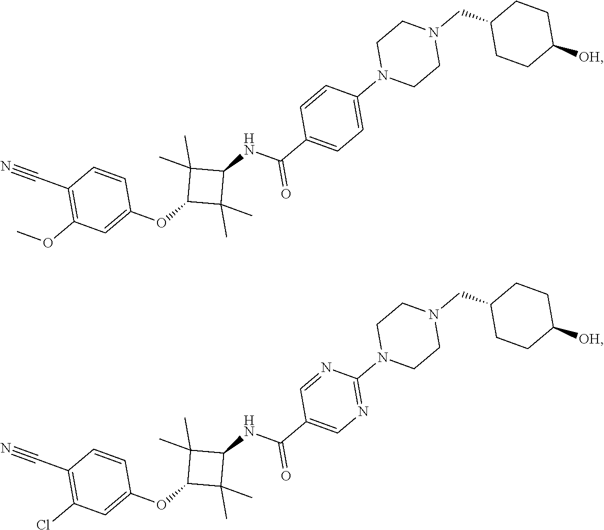 [SMILES: COc1cc(O[C@H]2C(C)(C)[C@H](NC(=O)c3ccc(N4CCN(C[C@H]5CC[C@H](O)CC5)CC4)cc3)C2(C)C)ccc1C#N ; CC1(C)[C@H](NC(=O)c2cnc(N3CCN(C[C@H]4CC[C@H](O)CC4)CC3)nc2)C(C)(C)[C@H]1Oc1ccc(C#N)c(Cl)c1]

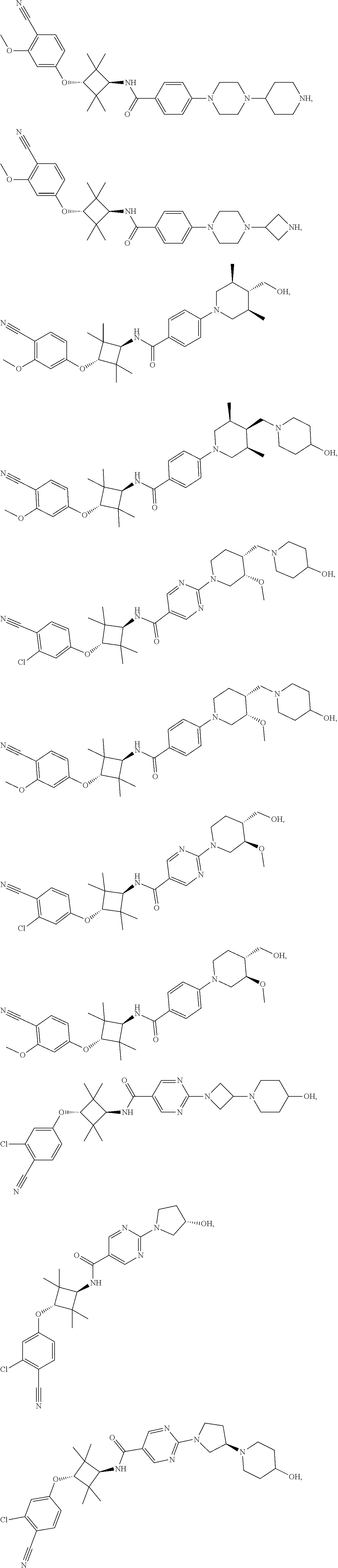

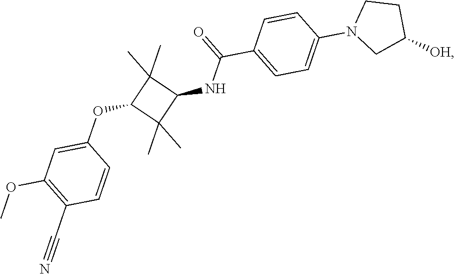 [SMILES: COc1cc(O[C@H]2C(C)(C)[C@H](NC(=O)c3ccc(N4CC[C@H](O)C4)cc3)C2(C)C)ccc1C#N]

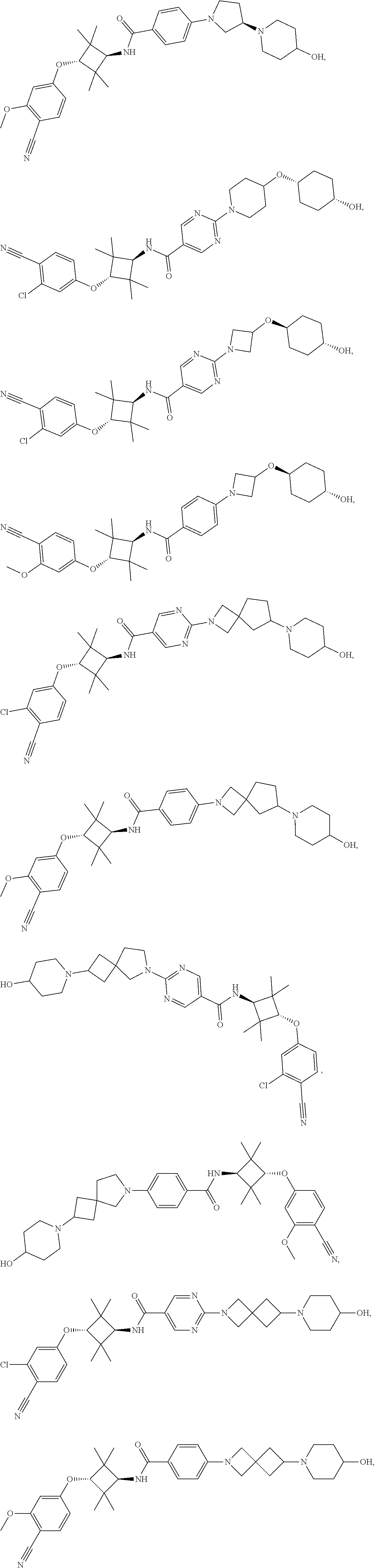

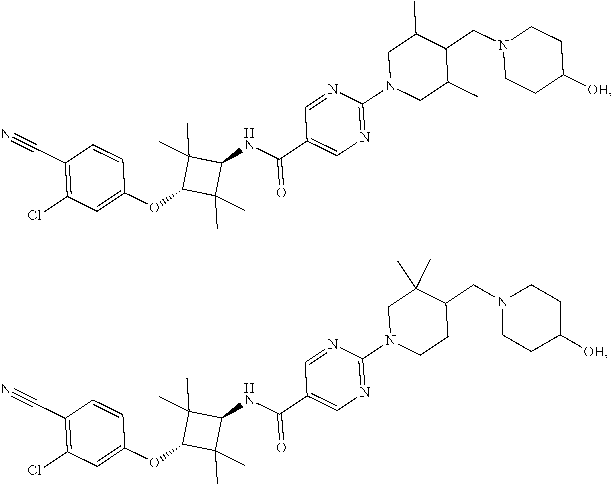 [SMILES: CC1CN(c2ncc(C(=O)N[C@H]3C(C)(C)[C@H](Oc4ccc(C#N)c(Cl)c4)C3(C)C)cn2)CC(C)C1CN1CCC(O)CC1 ; CC1(C)CN(c2ncc(C(=O)N[C@H]3C(C)(C)[C@H](Oc4ccc(C#N)c(Cl)c4)C3(C)C)cn2)CCC1CN1CCC(O)CC1]

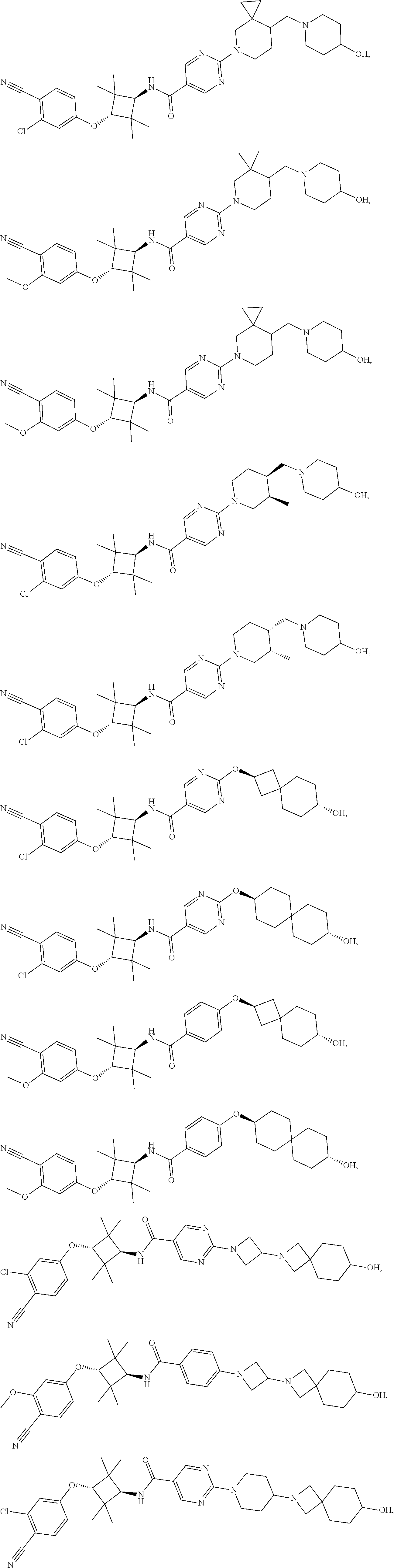

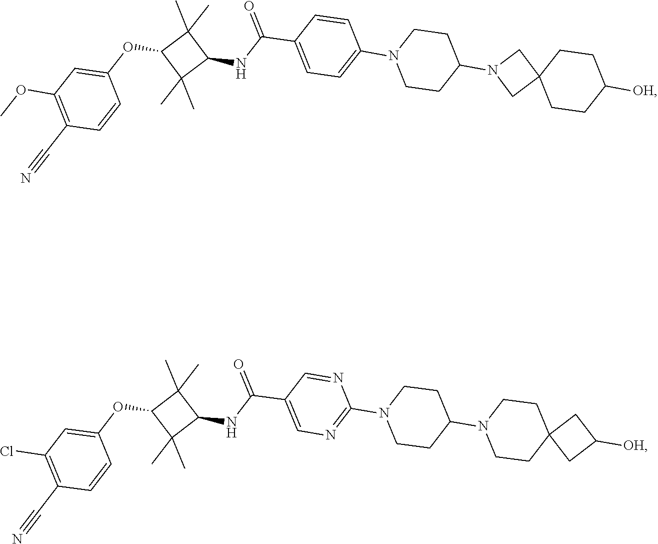 [SMILES: COc1cc(O[C@H]2C(C)(C)[C@H](NC(=O)c3ccc(N4CCC(N5CC6(CCC(O)CC6)C5)CC4)cc3)C2(C)C)ccc1C#N ; CC1(C)[C@H](NC(=O)c2cnc(N3CCC(N4CCC5(CC4)CC(O)C5)CC3)nc2)C(C)(C)[C@H]1Oc1ccc(C#N)c(Cl)c1]

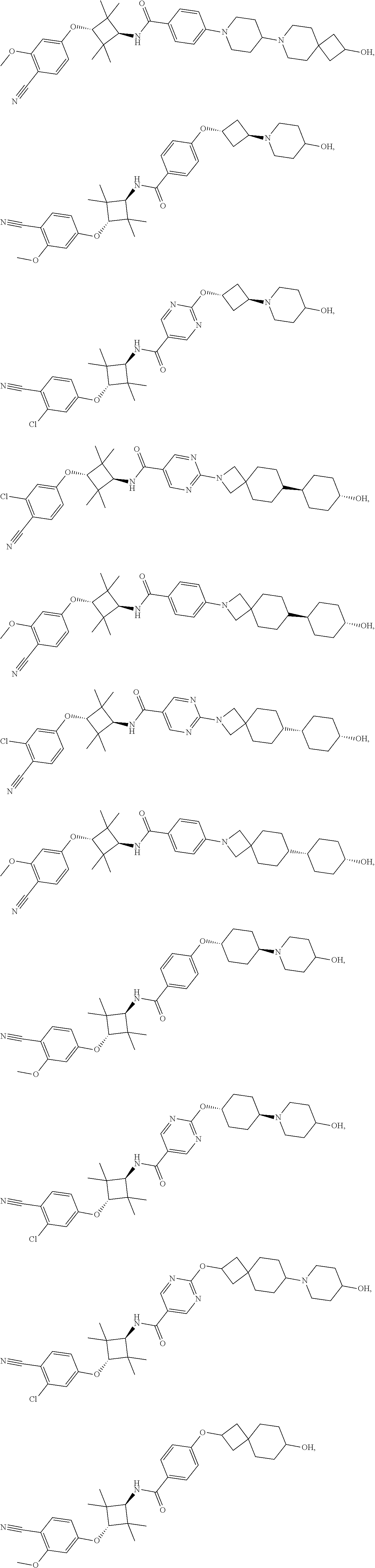

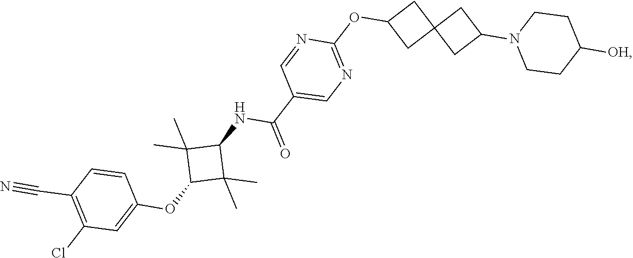 [SMILES: CC1(C)[C@H](NC(=O)c2cnc(OC3CC4(C3)CC(N3CCC(O)CC3)C4)nc2)C(C)(C)[C@H]1Oc1ccc(C#N)c(Cl)c1]

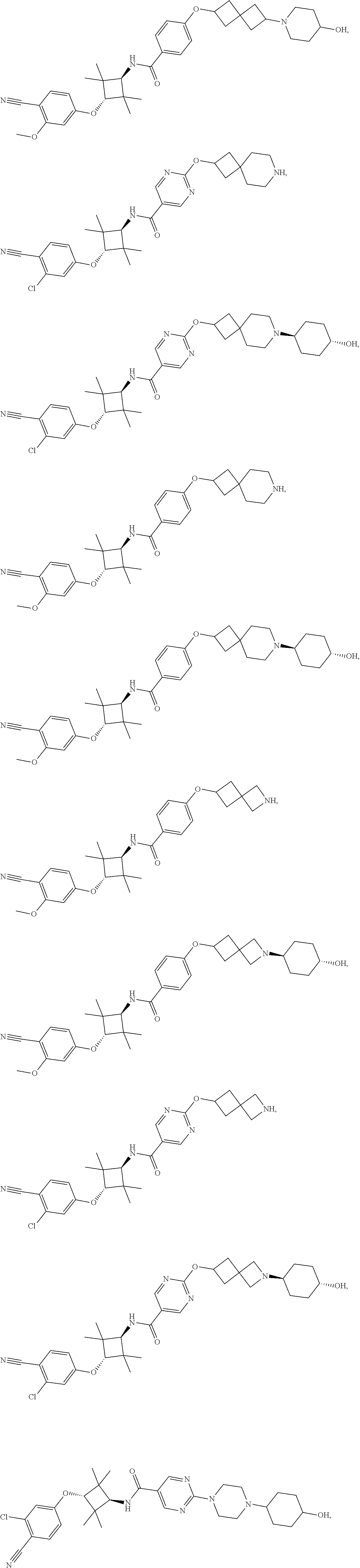

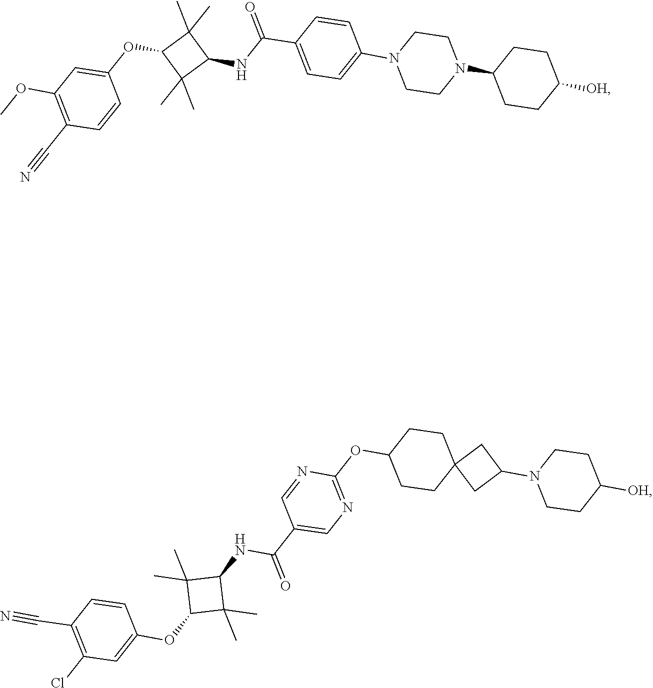 [SMILES: COc1cc(O[C@H]2C(C)(C)[C@H](NC(=O)c3ccc(N4CCN([C@H]5CC[C@H](O)CC5)CC4)cc3)C2(C)C)ccc1C#N ; CC1(C)[C@H](NC(=O)c2cnc(OC3CCC4(CC3)CC(N3CCC(O)CC3)C4)nc2)C(C)(C)[C@H]1Oc1ccc(C#N)c(Cl)c1]

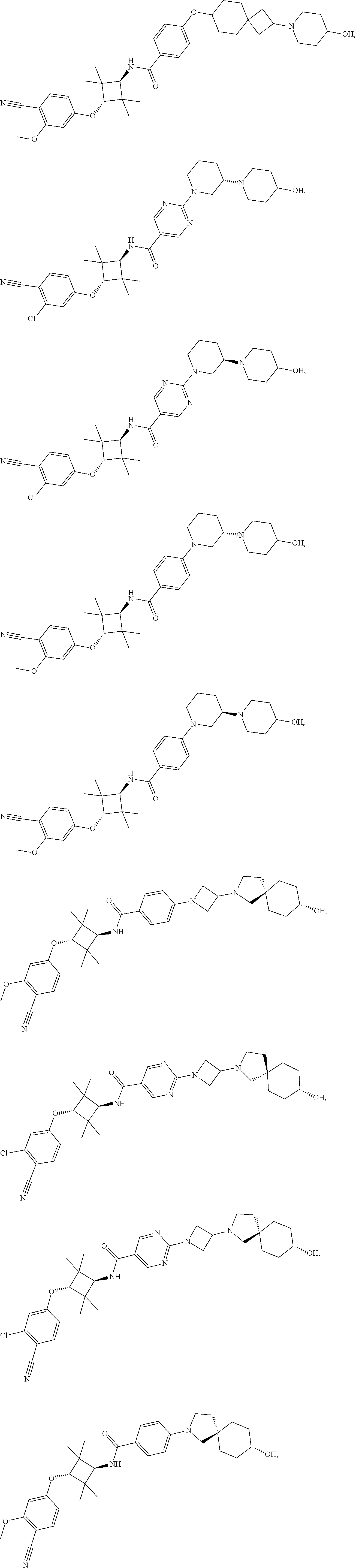

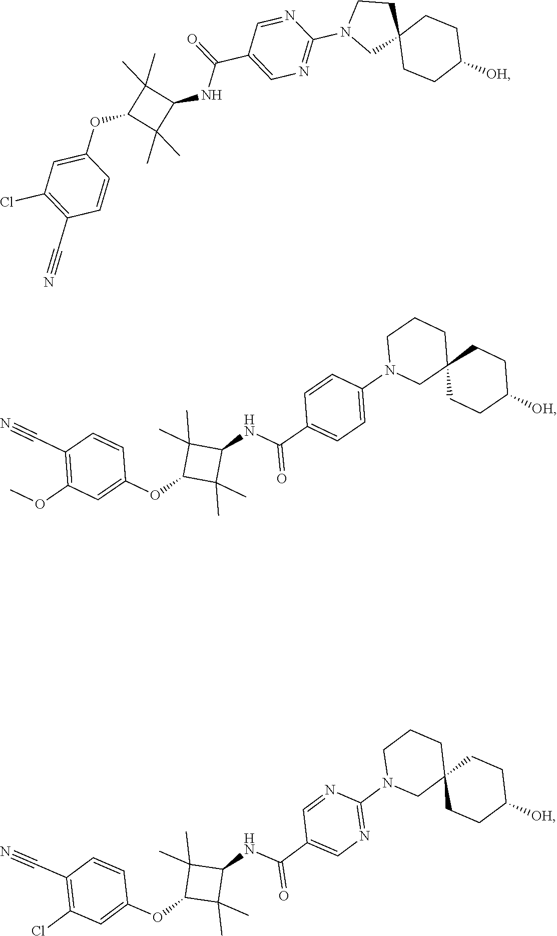 [SMILES: CC1(C)[C@H](NC(=O)c2cnc(N3CC[C@]4(CC[C@H](O)CC4)C3)nc2)C(C)(C)[C@H]1Oc1ccc(C#N)c(Cl)c1 ; COc1cc(O[C@H]2C(C)(C)[C@H](NC(=O)c3ccc(N4CCC[C@]5(CC[C@@H](O)CC5)C4)cc3)C2(C)C)ccc1C#N ; CC1(C)[C@H](NC(=O)c2cnc(N3CCC[C@]4(CC[C@H](O)CC4)C3)nc2)C(C)(C)[C@H]1Oc1ccc(C#N)c(Cl)c1]

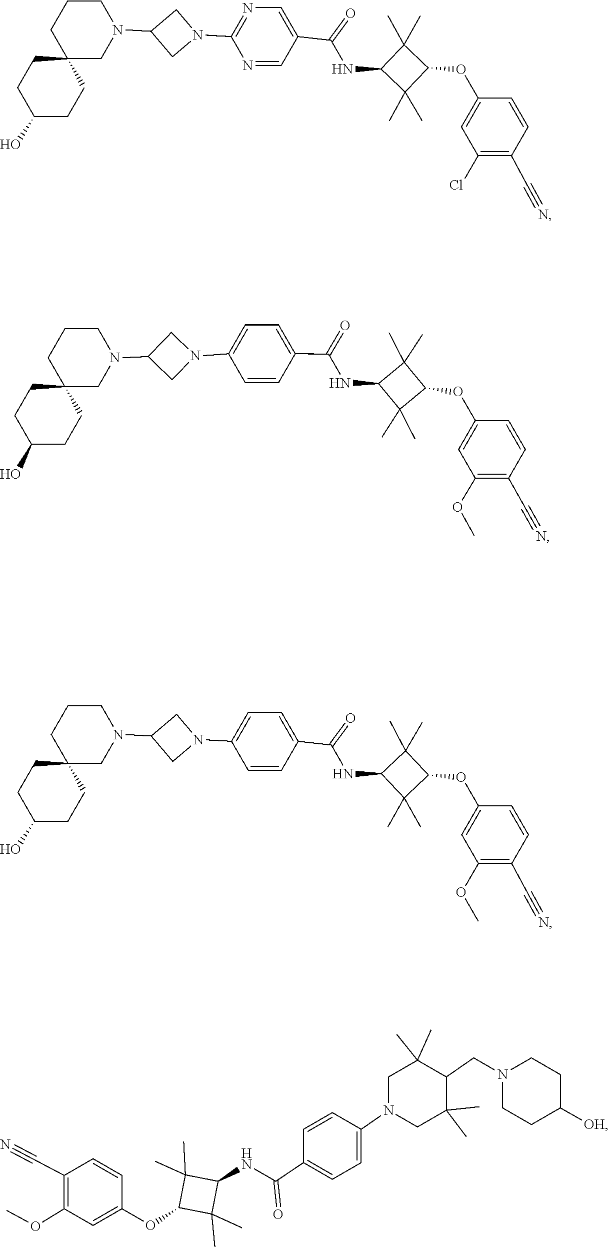 [SMILES: CC1(C)[C@H](NC(=O)c2cnc(N3CC(N4CCC[C@]5(CC[C@@H](O)CC5)C4)C3)nc2)C(C)(C)[C@H]1Oc1ccc(C#N)c(Cl)c1 ; COc1cc(O[C@H]2C(C)(C)[C@H](NC(=O)c3ccc(N4CC(N5CCC[C@]6(CC[C@H](O)CC6)C5)C4)cc3)C2(C)C)ccc1C#N ; COc1cc(O[C@H]2C(C)(C)[C@H](NC(=O)c3ccc(N4CC(N5CCC[C@]6(CC[C@@H](O)CC6)C5)C4)cc3)C2(C)C)ccc1C#N ; COc1cc(O[C@H]2C(C)(C)[C@H](NC(=O)c3ccc(N4CC(C)(C)C(CN5CCC(O)CC5)C(C)(C)C4)cc3)C2(C)C)ccc1C#N]

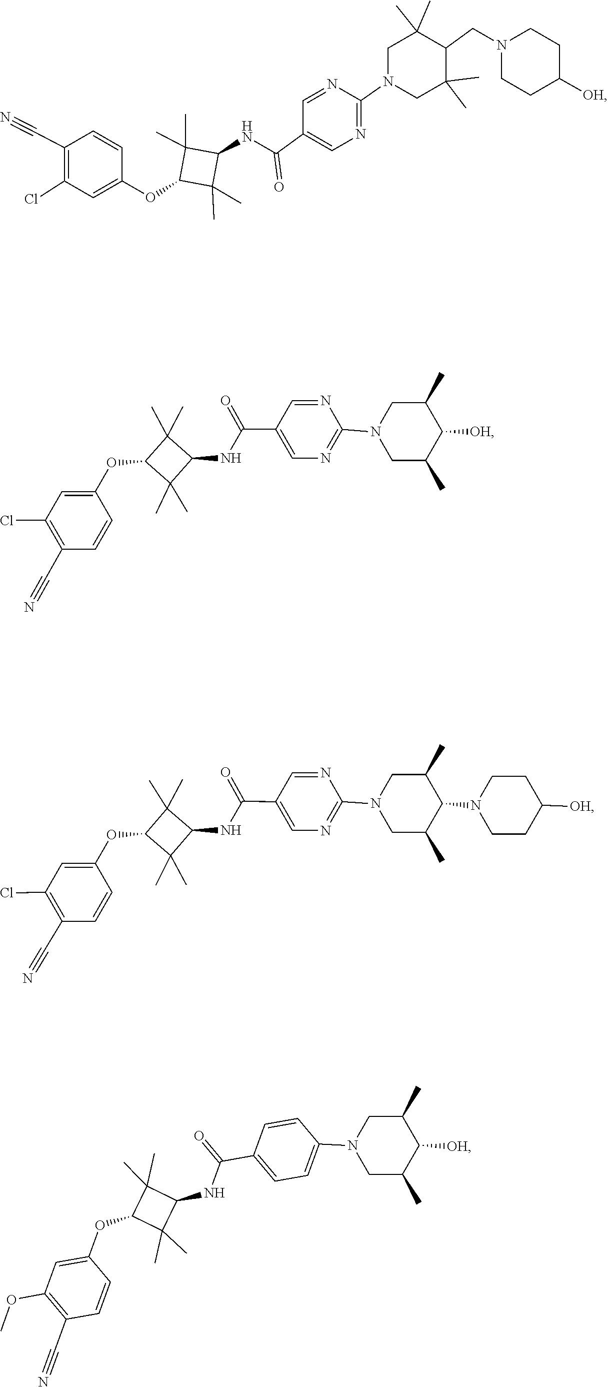 [SMILES: CC1(C)CN(c2ncc(C(=O)N[C@H]3C(C)(C)[C@H](Oc4ccc(C#N)c(Cl)c4)C3(C)C)cn2)CC(C)(C)C1CN1CCC(O)CC1 ; C[C@@H]1CN(c2ncc(C(=O)N[C@H]3C(C)(C)[C@H](Oc4ccc(C#N)c(Cl)c4)C3(C)C)cn2)C[C@H](C)[C@H]1O ; C[C@@H]1CN(c2ncc(C(=O)N[C@H]3C(C)(C)[C@H](Oc4ccc(C#N)c(Cl)c4)C3(C)C)cn2)C[C@H](C)[C@H]1N1CCC(O)CC1 ; COc1cc(O[C@H]2C(C)(C)[C@H](NC(=O)c3ccc(N4C[C@@H](C)[C@H](O)[C@@H](C)C4)cc3)C2(C)C)ccc1C#N]

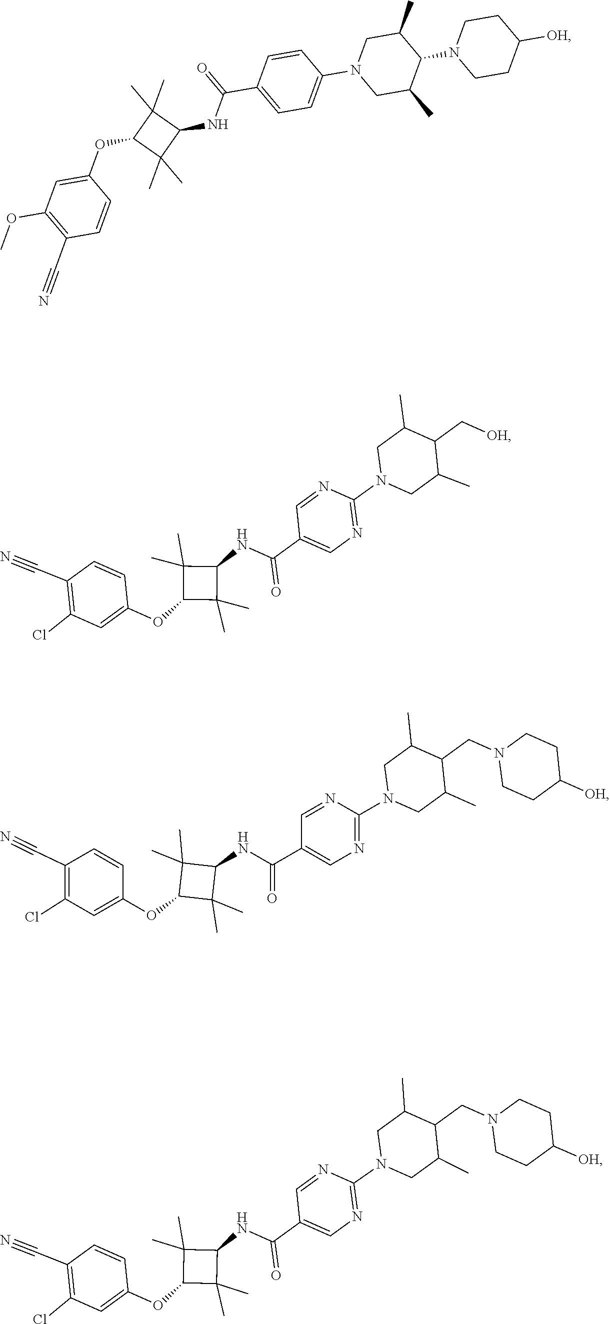 [SMILES: COc1cc(O[C@H]2C(C)(C)[C@H](NC(=O)c3ccc(N4C[C@@H](C)[C@H](N5CCC(O)CC5)[C@@H](C)C4)cc3)C2(C)C)ccc1C#N ; CC1CN(c2ncc(C(=O)N[C@H]3C(C)(C)[C@H](Oc4ccc(C#N)c(Cl)c4)C3(C)C)cn2)CC(C)C1CO ; CC1CN(c2ncc(C(=O)N[C@H]3C(C)(C)[C@H](Oc4ccc(C#N)c(Cl)c4)C3(C)C)cn2)CC(C)C1CN1CCC(O)CC1 ; CC1CN(c2ncc(C(=O)N[C@H]3C(C)(C)[C@H](Oc4ccc(C#N)c(Cl)c4)C3(C)C)cn2)CC(C)C1CN1CCC(O)CC1]

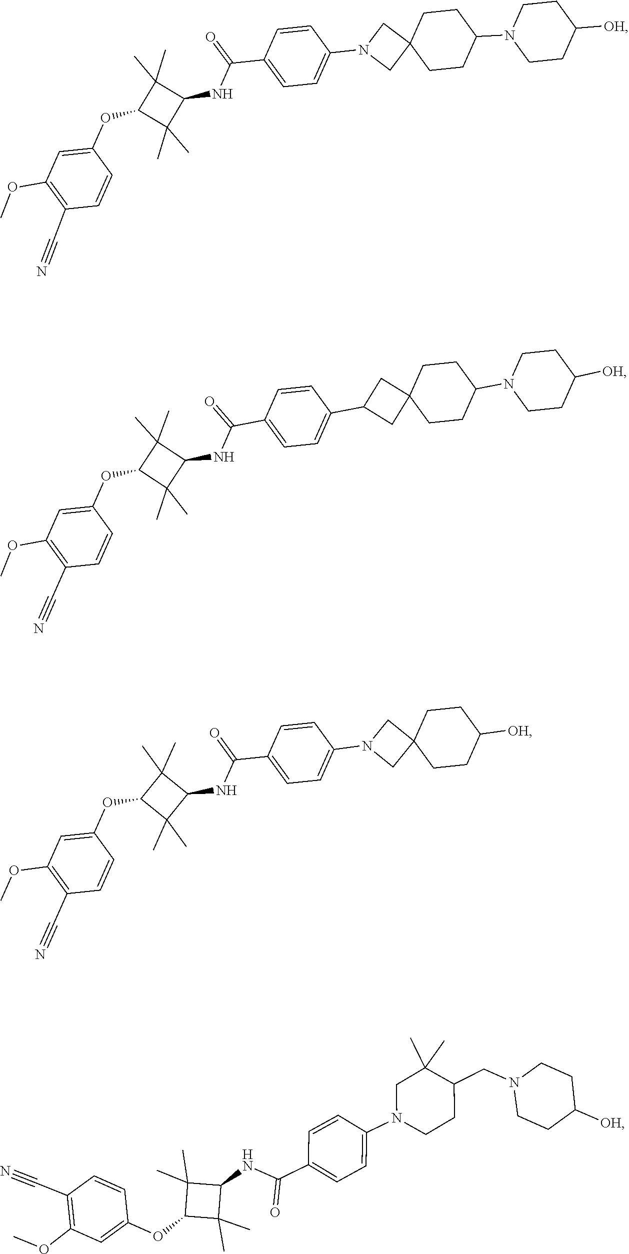 [SMILES: COc1cc(O[C@H]2C(C)(C)[C@H](NC(=O)c3ccc(N4CC5(CCC(N6CCC(O)CC6)CC5)C4)cc3)C2(C)C)ccc1C#N ; COc1cc(O[C@H]2C(C)(C)[C@H](NC(=O)c3ccc(C4CC5(CCC(N6CCC(O)CC6)CC5)C4)cc3)C2(C)C)ccc1C#N ; COc1cc(O[C@H]2C(C)(C)[C@H](NC(=O)c3ccc(N4CC5(CCC(O)CC5)C4)cc3)C2(C)C)ccc1C#N ; COc1cc(O[C@H]2C(C)(C)[C@H](NC(=O)c3ccc(N4CCC(CN5CCC(O)CC5)C(C)(C)C4)cc3)C2(C)C)ccc1C#N]

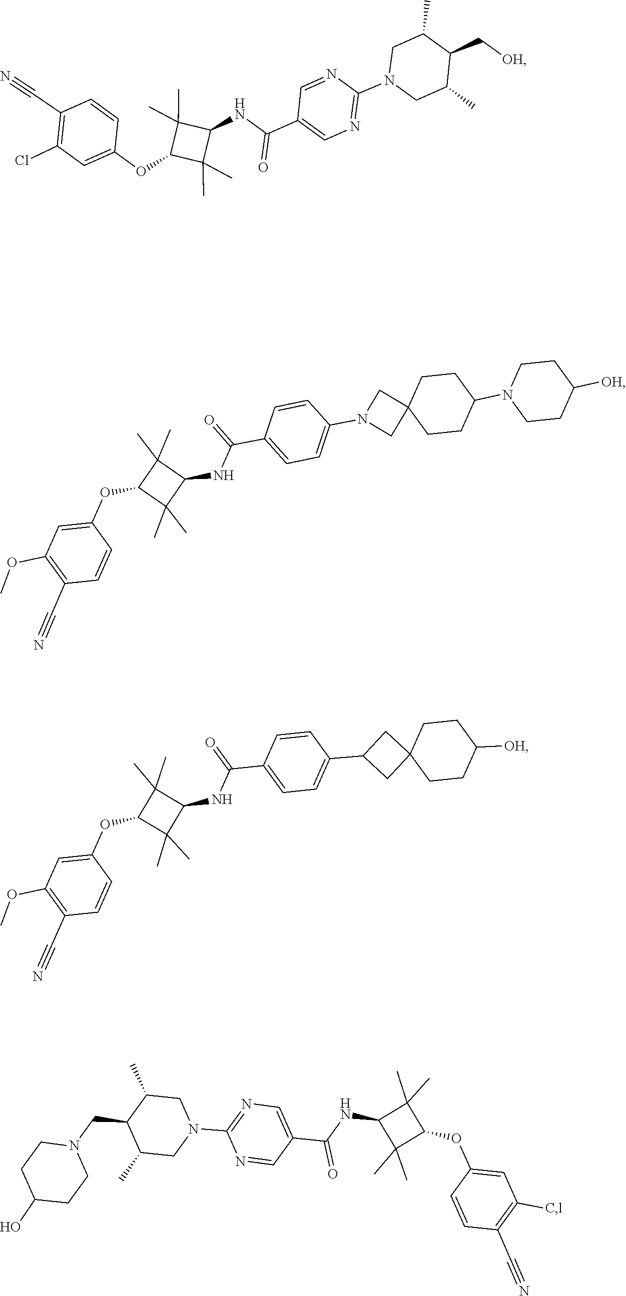

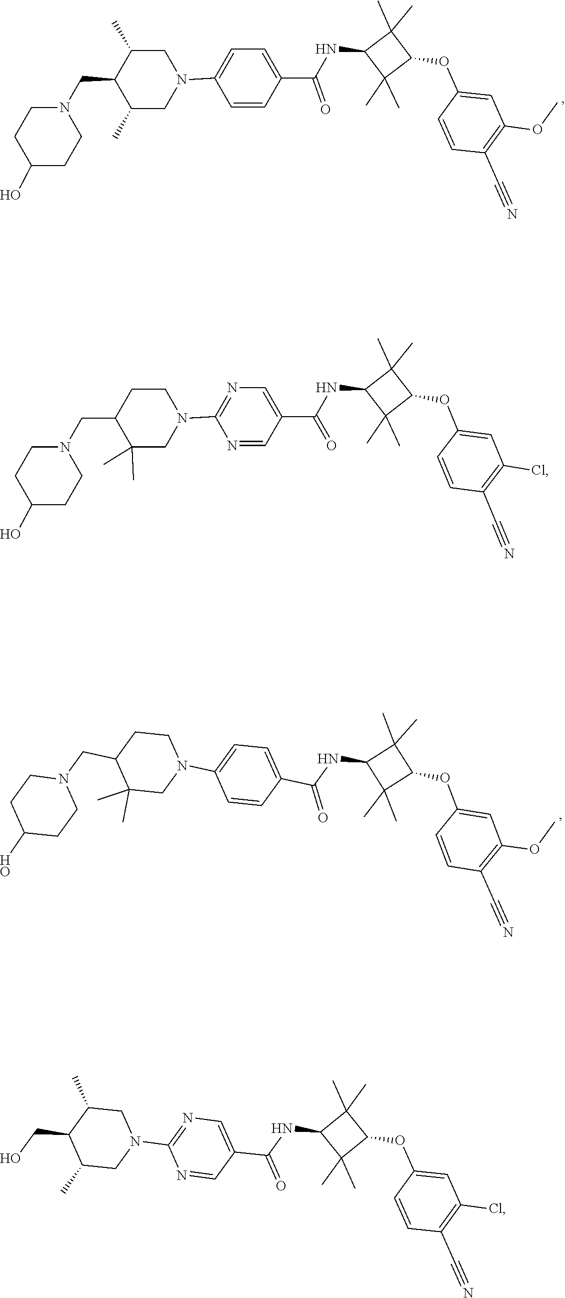 [SMILES: COc1cc(O[C@H]2C(C)(C)[C@H](NC(=O)c3ccc(N4C[C@@H](C)[C@H](CN5CCC(O)CC5)[C@@H](C)C4)cc3)C2(C)C)ccc1C#N ; CC1(C)CN(c2ncc(C(=O)N[C@H]3C(C)(C)[C@H](Oc4ccc(C#N)c(Cl)c4)C3(C)C)cn2)CCC1CN1CCC(O)CC1 ; COc1cc(O[C@H]2C(C)(C)[C@H](NC(=O)c3ccc(N4CCC(CN5CCC(O)CC5)C(C)(C)C4)cc3)C2(C)C)ccc1C#N ; C[C@@H]1CN(c2ncc(C(=O)N[C@H]3C(C)(C)[C@H](Oc4ccc(C#N)c(Cl)c4)C3(C)C)cn2)C[C@H](C)[C@H]1CO]

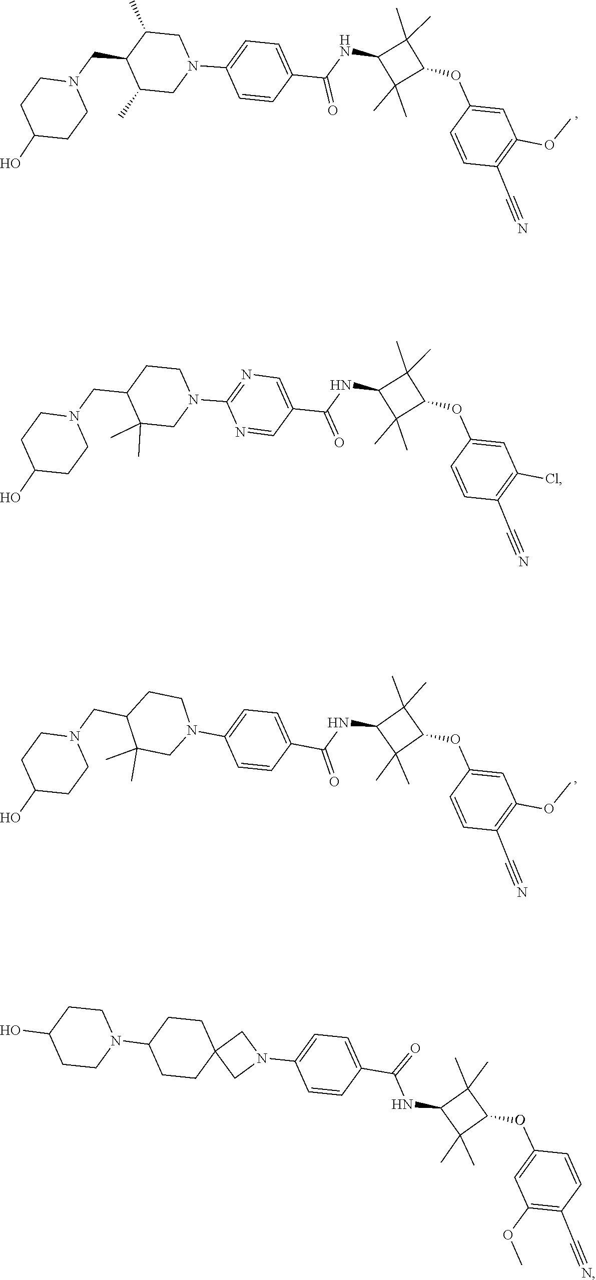 [SMILES: COc1cc(O[C@H]2C(C)(C)[C@H](NC(=O)c3ccc(N4C[C@@H](C)[C@H](CN5CCC(O)CC5)[C@@H](C)C4)cc3)C2(C)C)ccc1C#N ; CC1(C)CN(c2ncc(C(=O)N[C@H]3C(C)(C)[C@H](Oc4ccc(C#N)c(Cl)c4)C3(C)C)cn2)CCC1CN1CCC(O)CC1 ; COc1cc(O[C@H]2C(C)(C)[C@H](NC(=O)c3ccc(N4CCC(CN5CCC(O)CC5)C(C)(C)C4)cc3)C2(C)C)ccc1C#N ; COc1cc(O[C@H]2C(C)(C)[C@H](NC(=O)c3ccc(N4CC5(CCC(N6CCC(O)CC6)CC5)C4)cc3)C2(C)C)ccc1C#N]

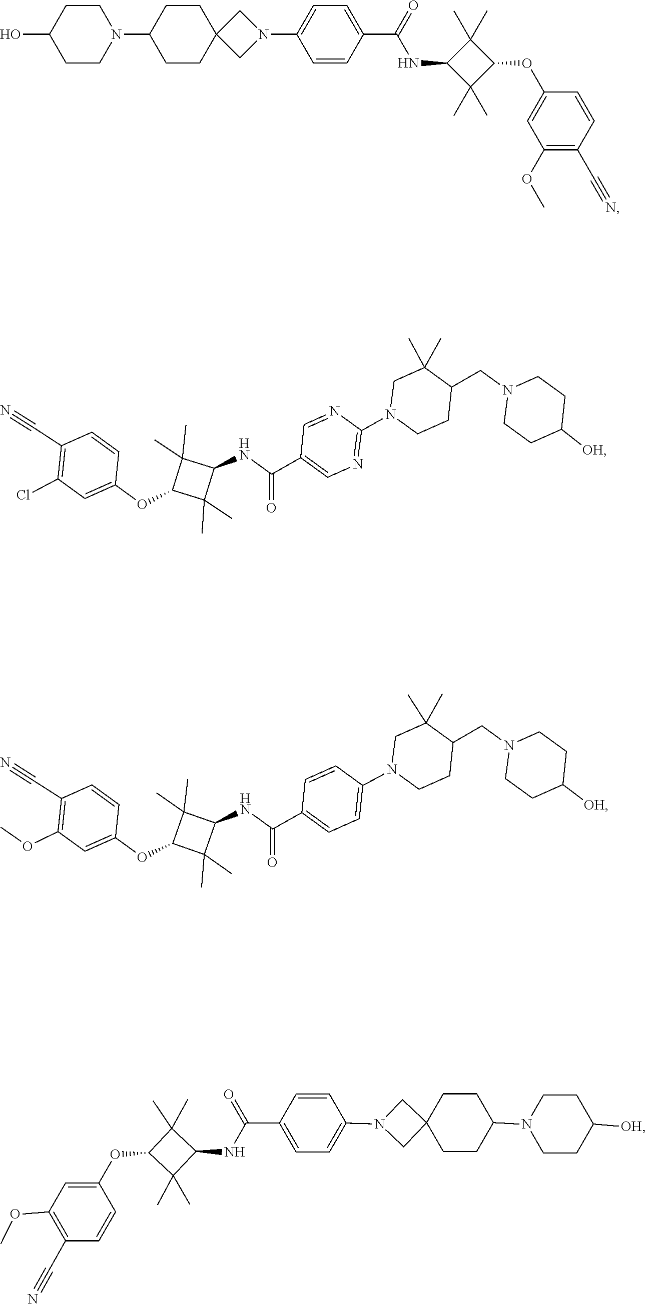 [SMILES: COc1cc(O[C@H]2C(C)(C)[C@H](NC(=O)c3ccc(N4CC5(CCC(N6CCC(O)CC6)CC5)C4)cc3)C2(C)C)ccc1C#N ; CC1(C)CN(c2ncc(C(=O)N[C@H]3C(C)(C)[C@H](Oc4ccc(C#N)c(Cl)c4)C3(C)C)cn2)CCC1CN1CCC(O)CC1 ; COc1cc(O[C@H]2C(C)(C)[C@H](NC(=O)c3ccc(N4CCC(CN5CCC(O)CC5)C(C)(C)C4)cc3)C2(C)C)ccc1C#N ; COc1cc(O[C@H]2C(C)(C)[C@H](NC(=O)c3ccc(N4CC5(CCC(N6CCC(O)CC6)CC5)C4)cc3)C2(C)C)ccc1C#N]

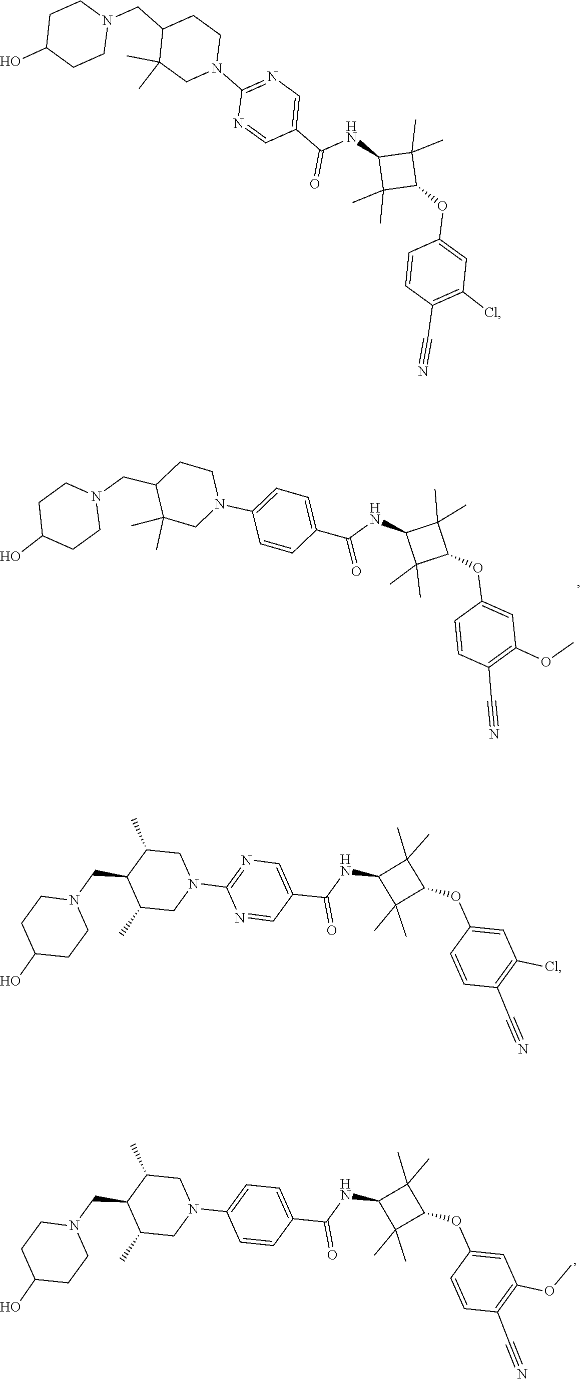 [SMILES: CC1(C)CN(c2ncc(C(=O)N[C@H]3C(C)(C)[C@H](Oc4ccc(C#N)c(Cl)c4)C3(C)C)cn2)CCC1CN1CCC(O)CC1 ; COc1cc(O[C@H]2C(C)(C)[C@H](NC(=O)c3ccc(N4CCC(CN5CCC(O)CC5)C(C)(C)C4)cc3)C2(C)C)ccc1C#N ; C[C@@H]1CN(c2ncc(C(=O)N[C@H]3C(C)(C)[C@H](Oc4ccc(C#N)c(Cl)c4)C3(C)C)cn2)C[C@H](C)[C@H]1CN1CCC(O)CC1 ; COc1cc(O[C@H]2C(C)(C)[C@H](NC(=O)c3ccc(N4C[C@@H](C)[C@H](CN5CCC(O)CC5)[C@@H](C)C4)cc3)C2(C)C)ccc1C#N]

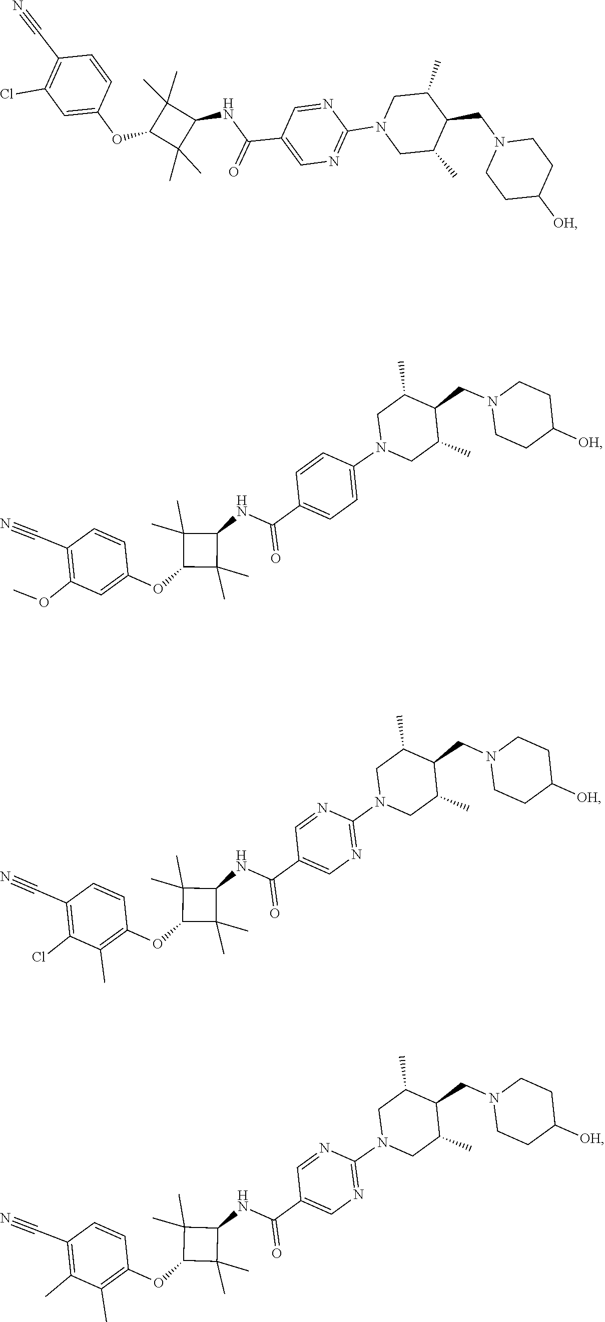 [SMILES: C[C@@H]1CN(c2ncc(C(=O)N[C@H]3C(C)(C)[C@H](Oc4ccc(C#N)c(Cl)c4)C3(C)C)cn2)C[C@H](C)[C@H]1CN1CCC(O)CC1 ; COc1cc(O[C@H]2C(C)(C)[C@H](NC(=O)c3ccc(N4C[C@@H](C)[C@H](CN5CCC(O)CC5)[C@@H](C)C4)cc3)C2(C)C)ccc1C#N ; Cc1c(O[C@H]2C(C)(C)[C@H](NC(=O)c3cnc(N4C[C@@H](C)[C@H](CN5CCC(O)CC5)[C@@H](C)C4)nc3)C2(C)C)ccc(C#N)c1Cl ; Cc1c(C#N)ccc(O[C@H]2C(C)(C)[C@H](NC(=O)c3cnc(N4C[C@@H](C)[C@H](CN5CCC(O)CC5)[C@@H](C)C4)nc3)C2(C)C)c1C]

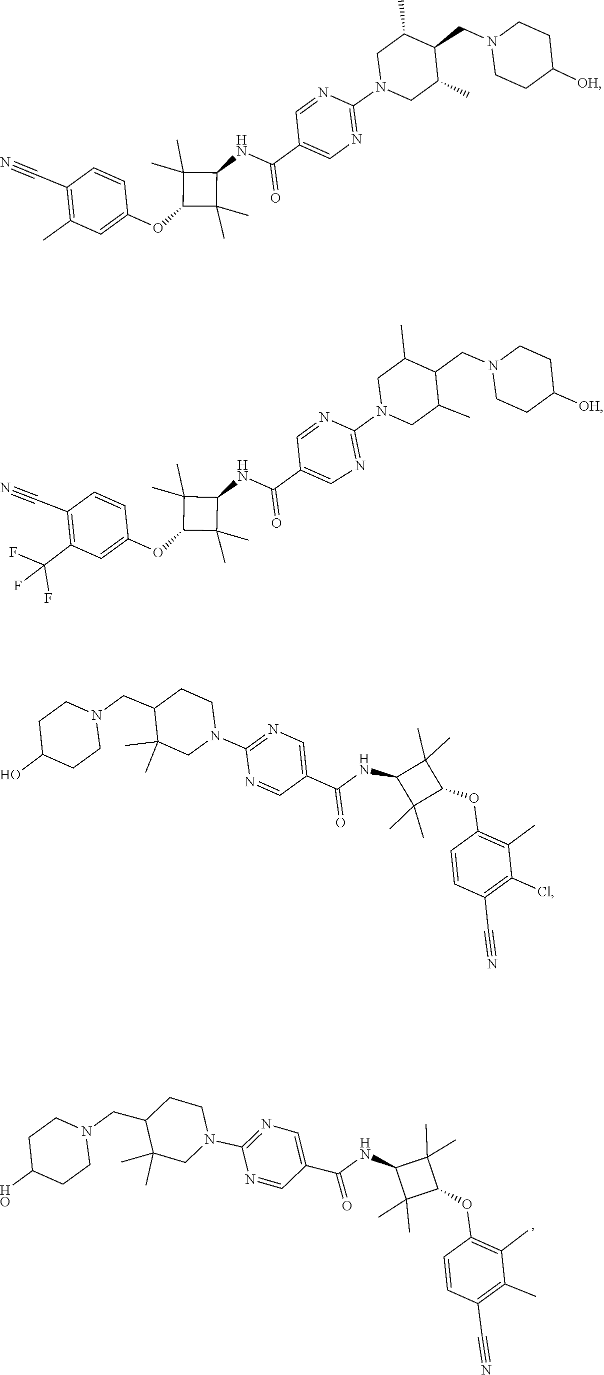 [SMILES: Cc1cc(O[C@H]2C(C)(C)[C@H](NC(=O)c3cnc(N4C[C@@H](C)[C@H](CN5CCC(O)CC5)[C@@H](C)C4)nc3)C2(C)C)ccc1C#N ; CC1CN(c2ncc(C(=O)N[C@H]3C(C)(C)[C@H](Oc4ccc(C#N)c(C(F)(F)F)c4)C3(C)C)cn2)CC(C)C1CN1CCC(O)CC1 ; Cc1c(O[C@H]2C(C)(C)[C@H](NC(=O)c3cnc(N4CCC(CN5CCC(O)CC5)C(C)(C)C4)nc3)C2(C)C)ccc(C#N)c1Cl ; Cc1c(C#N)ccc(O[C@H]2C(C)(C)[C@H](NC(=O)c3cnc(N4CCC(CN5CCC(O)CC5)C(C)(C)C4)nc3)C2(C)C)c1C]

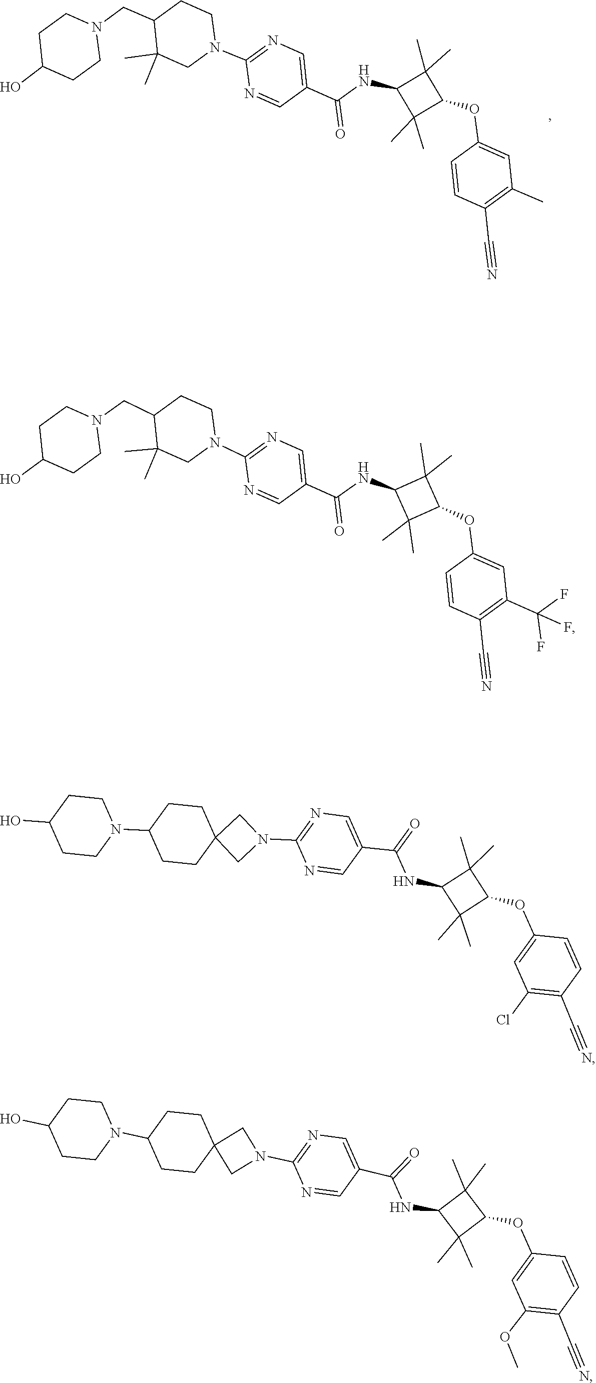 [SMILES: Cc1cc(O[C@H]2C(C)(C)[C@H](NC(=O)c3cnc(N4CCC(CN5CCC(O)CC5)C(C)(C)C4)nc3)C2(C)C)ccc1C#N ; CC1(C)CN(c2ncc(C(=O)N[C@H]3C(C)(C)[C@H](Oc4ccc(C#N)c(C(F)(F)F)c4)C3(C)C)cn2)CCC1CN1CCC(O)CC1 ; CC1(C)[C@H](NC(=O)c2cnc(N3CC4(CCC(N5CCC(O)CC5)CC4)C3)nc2)C(C)(C)[C@H]1Oc1ccc(C#N)c(Cl)c1 ; COc1cc(O[C@H]2C(C)(C)[C@H](NC(=O)c3cnc(N4CC5(CCC(N6CCC(O)CC6)CC5)C4)nc3)C2(C)C)ccc1C#N]

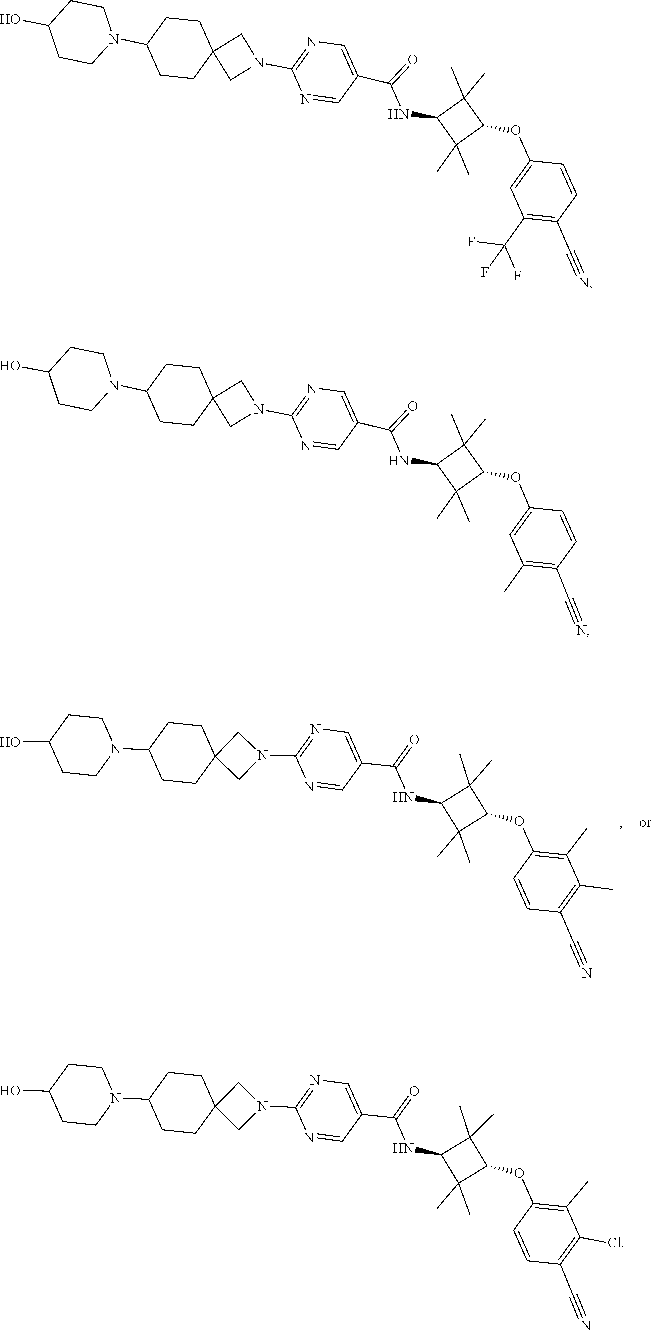 [SMILES: CC1(C)[C@H](NC(=O)c2cnc(N3CC4(CCC(N5CCC(O)CC5)CC4)C3)nc2)C(C)(C)[C@H]1Oc1ccc(C#N)c(C(F)(F)F)c1 ; Cc1cc(O[C@H]2C(C)(C)[C@H](NC(=O)c3cnc(N4CC5(CCC(N6CCC(O)CC6)CC5)C4)nc3)C2(C)C)ccc1C#N ; Cc1c(C#N)ccc(O[C@H]2C(C)(C)[C@H](NC(=O)c3cnc(N4CC5(CCC(N6CCC(O)CC6)CC5)C4)nc3)C2(C)C)c1C ; Cc1c(O[C@H]2C(C)(C)[C@H](NC(=O)c3cnc(N4CC5(CCC(N6CCC(O)CC6)CC5)C4)nc3)C2(C)C)ccc(C#N)c1Cl]

Embodiment 126. A compound, or a pharmaceutically acceptable salt or solvate thereof, that has one of the following structures:

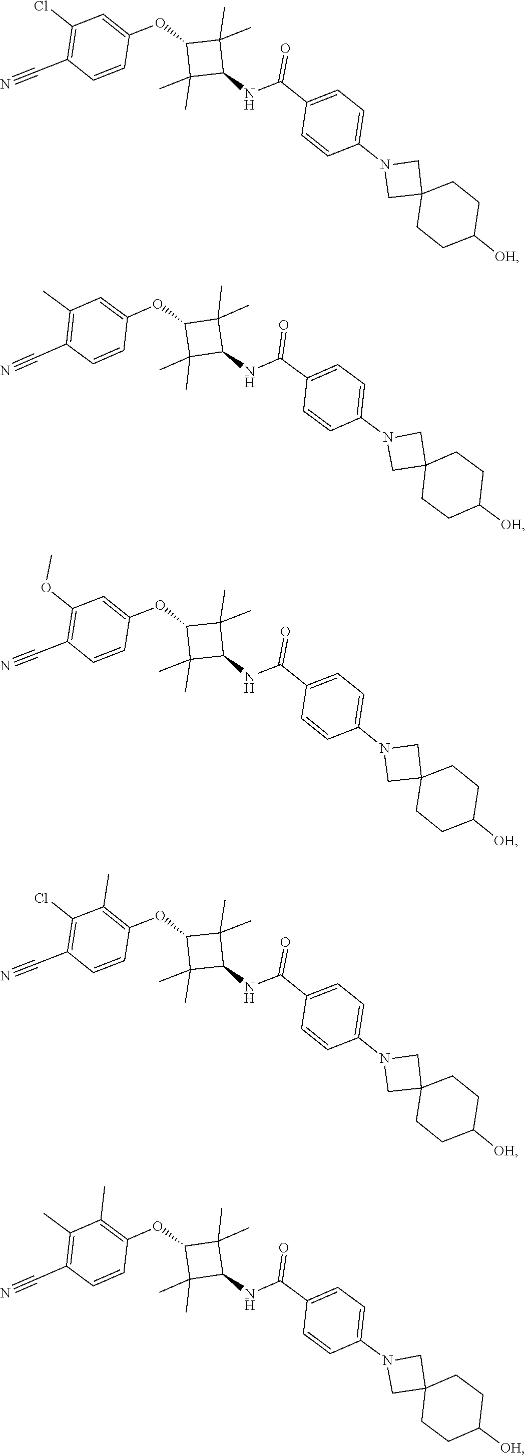 [SMILES: CC1(C)[C@H](NC(=O)c2ccc(N3CC4(CCC(O)CC4)C3)cc2)C(C)(C)[C@H]1Oc1ccc(C#N)c(Cl)c1 ; Cc1cc(O[C@H]2C(C)(C)[C@H](NC(=O)c3ccc(N4CC5(CCC(O)CC5)C4)cc3)C2(C)C)ccc1C#N ; COc1cc(O[C@H]2C(C)(C)[C@H](NC(=O)c3ccc(N4CC5(CCC(O)CC5)C4)cc3)C2(C)C)ccc1C#N ; Cc1c(O[C@H]2C(C)(C)[C@H](NC(=O)c3ccc(N4CC5(CCC(O)CC5)C4)cc3)C2(C)C)ccc(C#N)c1Cl ; Cc1c(C#N)ccc(O[C@H]2C(C)(C)[C@H](NC(=O)c3ccc(N4CC5(CCC(O)CC5)C4)cc3)C2(C)C)c1C]

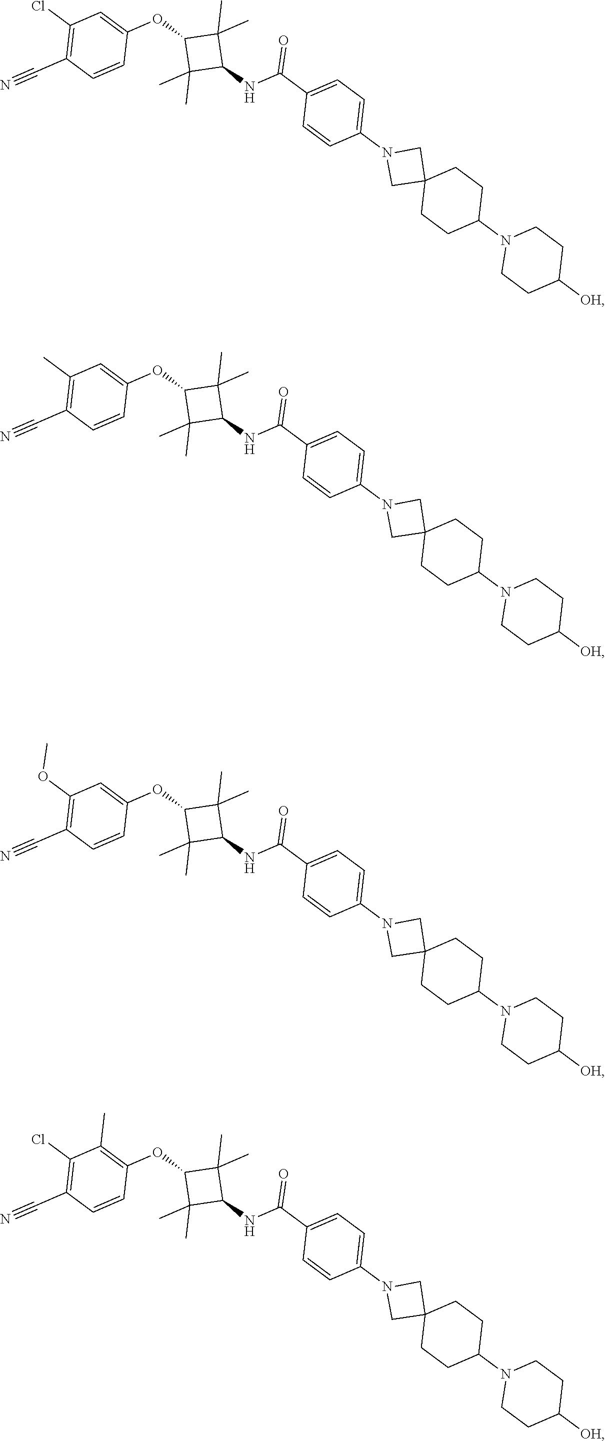 [SMILES: CC1(C)[C@H](NC(=O)c2ccc(N3CC4(CCC(N5CCC(O)CC5)CC4)C3)cc2)C(C)(C)[C@H]1Oc1ccc(C#N)c(Cl)c1 ; Cc1cc(O[C@H]2C(C)(C)[C@H](NC(=O)c3ccc(N4CC5(CCC(N6CCC(O)CC6)CC5)C4)cc3)C2(C)C)ccc1C#N ; COc1cc(O[C@H]2C(C)(C)[C@H](NC(=O)c3ccc(N4CC5(CCC(N6CCC(O)CC6)CC5)C4)cc3)C2(C)C)ccc1C#N ; Cc1c(O[C@H]2C(C)(C)[C@H](NC(=O)c3ccc(N4CC5(CCC(N6CCC(O)CC6)CC5)C4)cc3)C2(C)C)ccc(C#N)c1Cl]

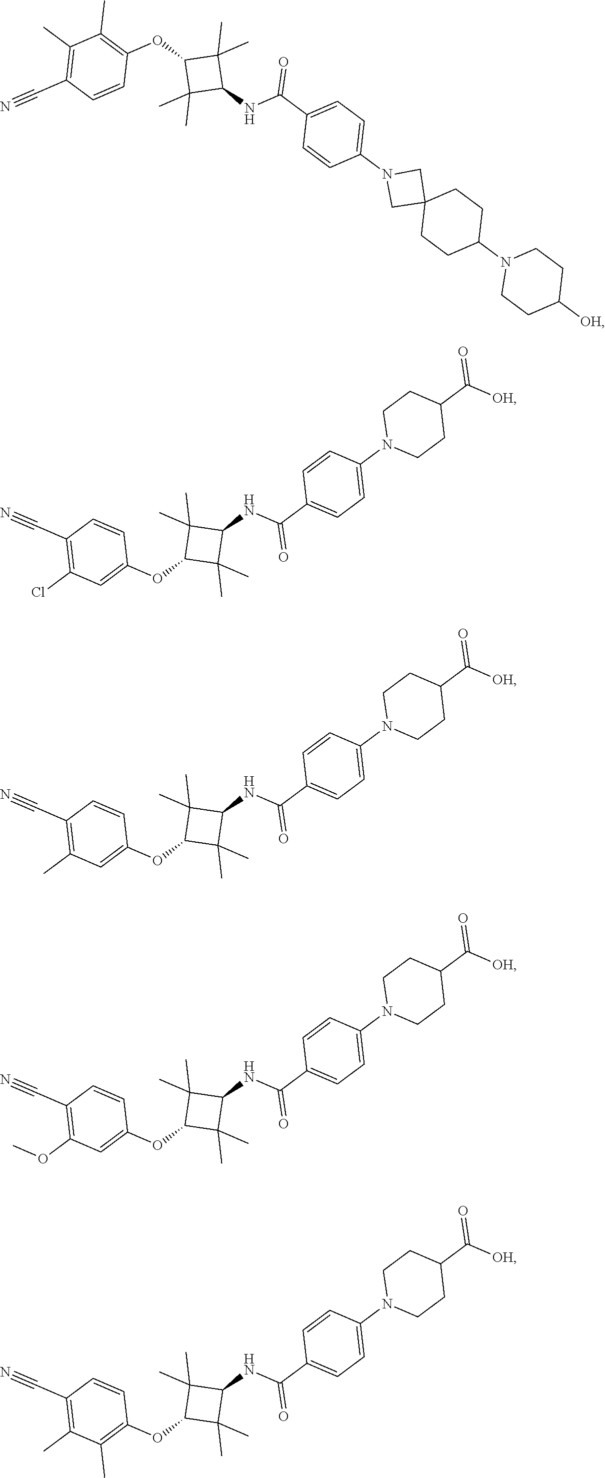 [SMILES: Cc1c(C#N)ccc(O[C@H]2C(C)(C)[C@H](NC(=O)c3ccc(N4CC5(CCC(N6CCC(O)CC6)CC5)C4)cc3)C2(C)C)c1C ; CC1(C)[C@H](NC(=O)c2ccc(N3CCC(C(=O)O)CC3)cc2)C(C)(C)[C@H]1Oc1ccc(C#N)c(Cl)c1 ; Cc1cc(O[C@H]2C(C)(C)[C@H](NC(=O)c3ccc(N4CCC(C(=O)O)CC4)cc3)C2(C)C)ccc1C#N ; COc1cc(O[C@H]2C(C)(C)[C@H](NC(=O)c3ccc(N4CCC(C(=O)O)CC4)cc3)C2(C)C)ccc1C#N ; Cc1c(C#N)ccc(O[C@H]2C(C)(C)[C@H](NC(=O)c3ccc(N4CCC(C(=O)O)CC4)cc3)C2(C)C)c1C]

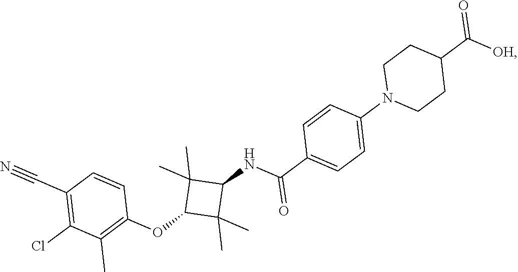 [SMILES: Cc1c(O[C@H]2C(C)(C)[C@H](NC(=O)c3ccc(N4CCC(C(=O)O)CC4)cc3)C2(C)C)ccc(C#N)c1Cl]

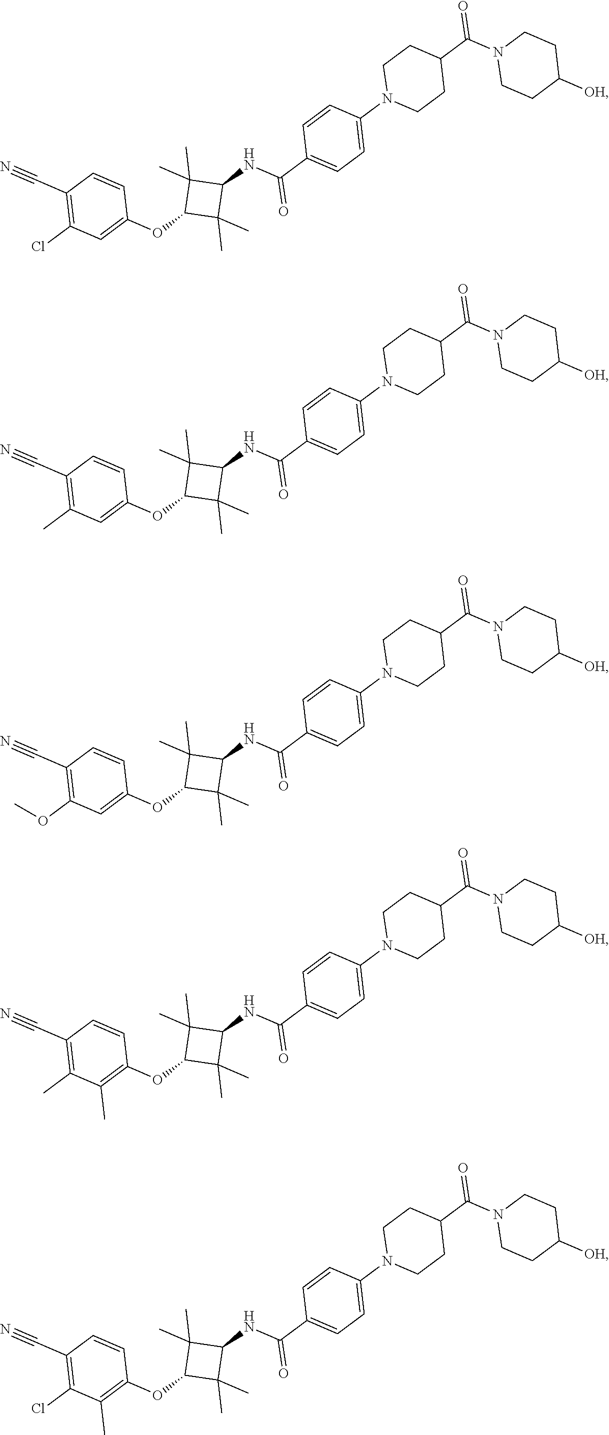 [SMILES: CC1(C)[C@H](NC(=O)c2ccc(N3CCC(C(=O)N4CCC(O)CC4)CC3)cc2)C(C)(C)[C@H]1Oc1ccc(C#N)c(Cl)c1 ; Cc1cc(O[C@H]2C(C)(C)[C@H](NC(=O)c3ccc(N4CCC(C(=O)N5CCC(O)CC5)CC4)cc3)C2(C)C)ccc1C#N ; COc1cc(O[C@H]2C(C)(C)[C@H](NC(=O)c3ccc(N4CCC(C(=O)N5CCC(O)CC5)CC4)cc3)C2(C)C)ccc1C#N ; Cc1c(C#N)ccc(O[C@H]2C(C)(C)[C@H](NC(=O)c3ccc(N4CCC(C(=O)N5CCC(O)CC5)CC4)cc3)C2(C)C)c1C ; Cc1c(O[C@H]2C(C)(C)[C@H](NC(=O)c3ccc(N4CCC(C(=O)N5CCC(O)CC5)CC4)cc3)C2(C)C)ccc(C#N)c1Cl]

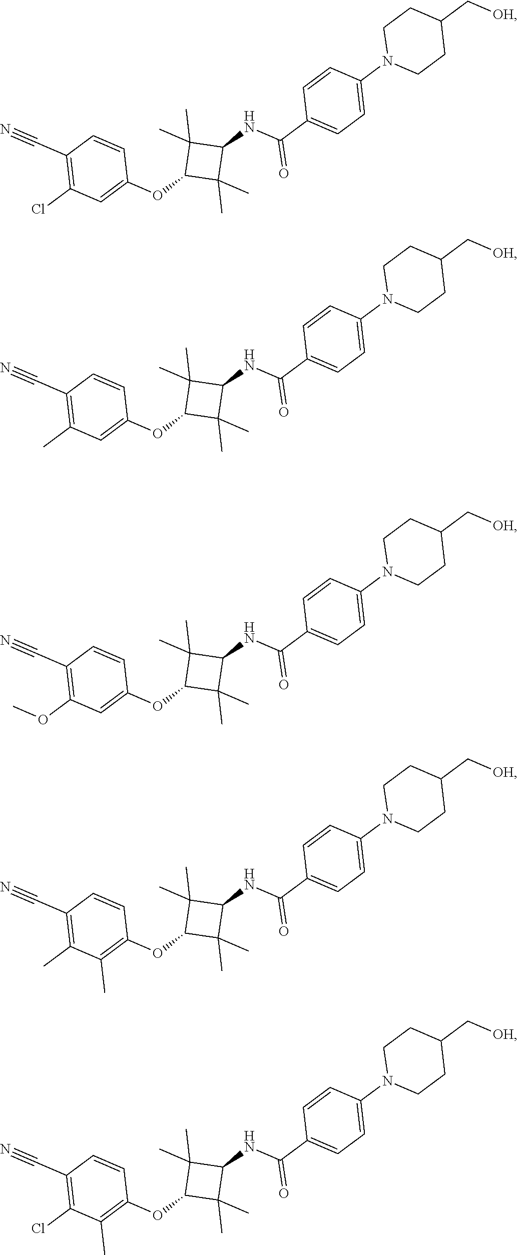 [SMILES: CC1(C)[C@H](NC(=O)c2ccc(N3CCC(CO)CC3)cc2)C(C)(C)[C@H]1Oc1ccc(C#N)c(Cl)c1 ; Cc1cc(O[C@H]2C(C)(C)[C@H](NC(=O)c3ccc(N4CCC(CO)CC4)cc3)C2(C)C)ccc1C#N ; COc1cc(O[C@H]2C(C)(C)[C@H](NC(=O)c3ccc(N4CCC(CO)CC4)cc3)C2(C)C)ccc1C#N ; Cc1c(C#N)ccc(O[C@H]2C(C)(C)[C@H](NC(=O)c3ccc(N4CCC(CO)CC4)cc3)C2(C)C)c1C ; Cc1c(O[C@H]2C(C)(C)[C@H](NC(=O)c3ccc(N4CCC(CO)CC4)cc3)C2(C)C)ccc(C#N)c1Cl]

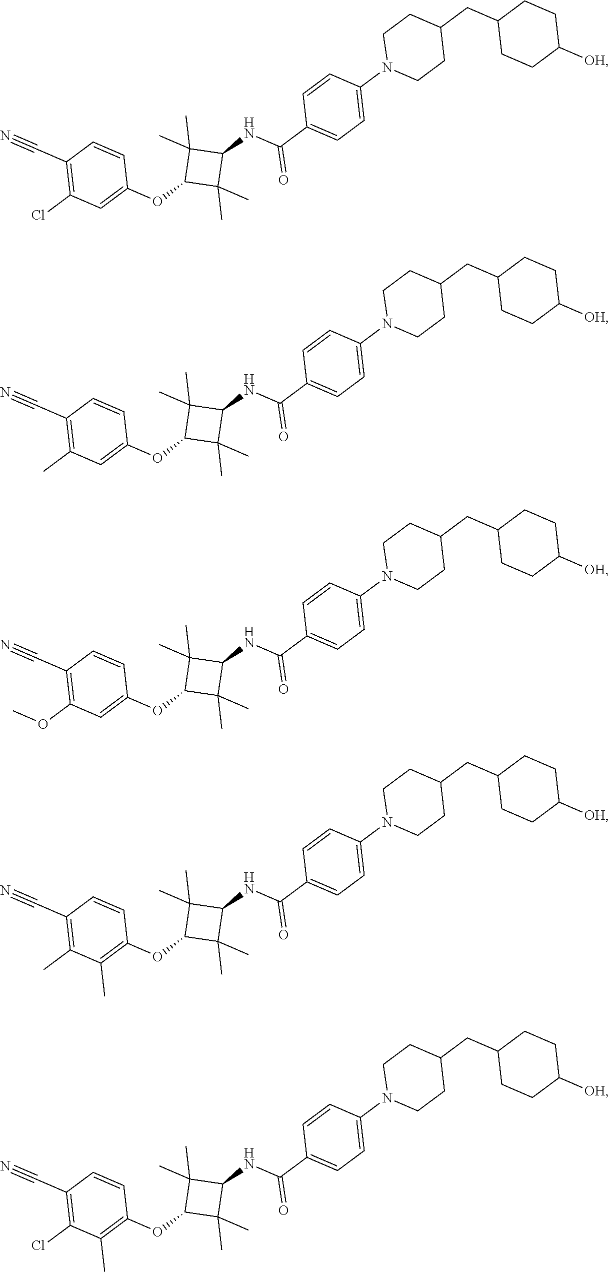 [SMILES: CC1(C)[C@H](NC(=O)c2ccc(N3CCC(CC4CCC(O)CC4)CC3)cc2)C(C)(C)[C@H]1Oc1ccc(C#N)c(Cl)c1 ; Cc1cc(O[C@H]2C(C)(C)[C@H](NC(=O)c3ccc(N4CCC(CC5CCC(O)CC5)CC4)cc3)C2(C)C)ccc1C#N ; COc1cc(O[C@H]2C(C)(C)[C@H](NC(=O)c3ccc(N4CCC(CC5CCC(O)CC5)CC4)cc3)C2(C)C)ccc1C#N ; Cc1c(C#N)ccc(O[C@H]2C(C)(C)[C@H](NC(=O)c3ccc(N4CCC(CC5CCC(O)CC5)CC4)cc3)C2(C)C)c1C ; Cc1c(O[C@H]2C(C)(C)[C@H](NC(=O)c3ccc(N4CCC(CC5CCC(O)CC5)CC4)cc3)C2(C)C)ccc(C#N)c1Cl]

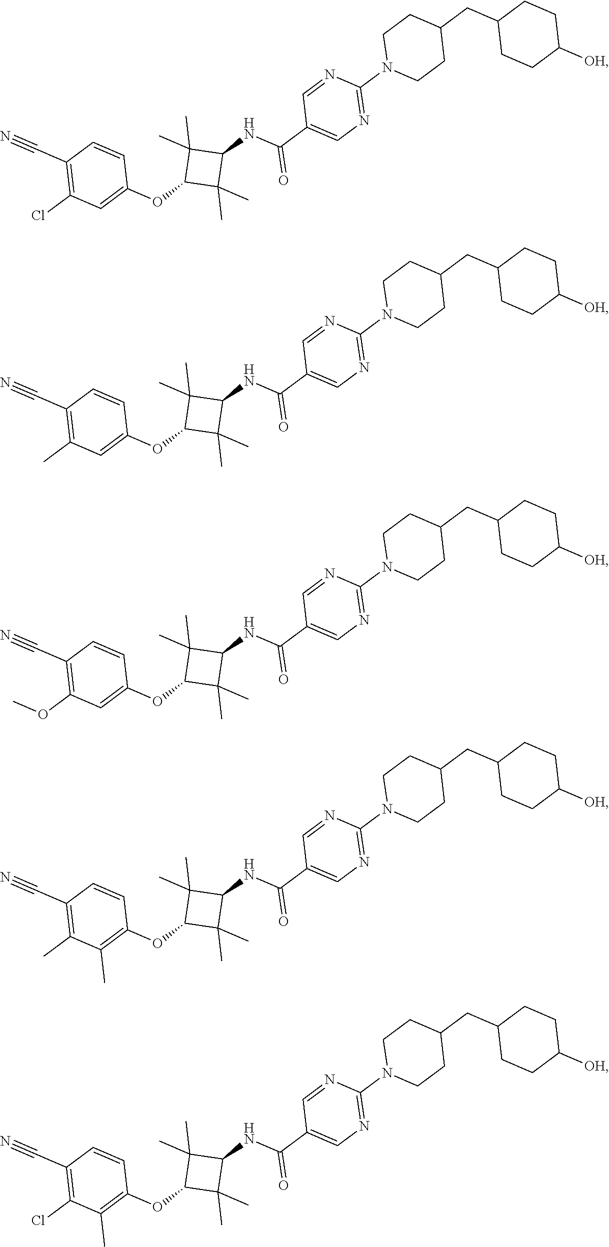 [SMILES: CC1(C)[C@H](NC(=O)c2cnc(N3CCC(CC4CCC(O)CC4)CC3)nc2)C(C)(C)[C@H]1Oc1ccc(C#N)c(Cl)c1 ; Cc1cc(O[C@H]2C(C)(C)[C@H](NC(=O)c3cnc(N4CCC(CC5CCC(O)CC5)CC4)nc3)C2(C)C)ccc1C#N ; COc1cc(O[C@H]2C(C)(C)[C@H](NC(=O)c3cnc(N4CCC(CC5CCC(O)CC5)CC4)nc3)C2(C)C)ccc1C#N ; Cc1c(C#N)ccc(O[C@H]2C(C)(C)[C@H](NC(=O)c3cnc(N4CCC(CC5CCC(O)CC5)CC4)nc3)C2(C)C)c1C ; Cc1c(O[C@H]2C(C)(C)[C@H](NC(=O)c3cnc(N4CCC(CC5CCC(O)CC5)CC4)nc3)C2(C)C)ccc(C#N)c1Cl]

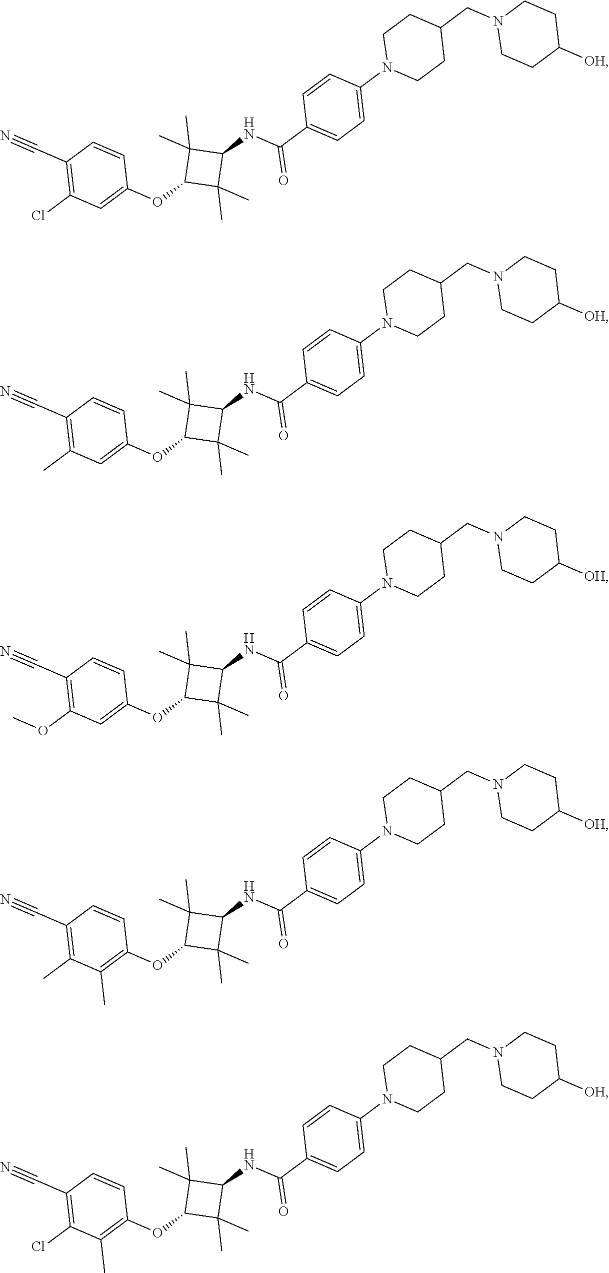 [SMILES: CC1(C)[C@H](NC(=O)c2ccc(N3CCC(CN4CCC(O)CC4)CC3)cc2)C(C)(C)[C@H]1Oc1ccc(C#N)c(Cl)c1 ; Cc1cc(O[C@H]2C(C)(C)[C@H](NC(=O)c3ccc(N4CCC(CN5CCC(O)CC5)CC4)cc3)C2(C)C)ccc1C#N ; COc1cc(O[C@H]2C(C)(C)[C@H](NC(=O)c3ccc(N4CCC(CN5CCC(O)CC5)CC4)cc3)C2(C)C)ccc1C#N ; Cc1c(C#N)ccc(O[C@H]2C(C)(C)[C@H](NC(=O)c3ccc(N4CCC(CN5CCC(O)CC5)CC4)cc3)C2(C)C)c1C ; Cc1c(O[C@H]2C(C)(C)[C@H](NC(=O)c3ccc(N4CCC(CN5CCC(O)CC5)CC4)cc3)C2(C)C)ccc(C#N)c1Cl]

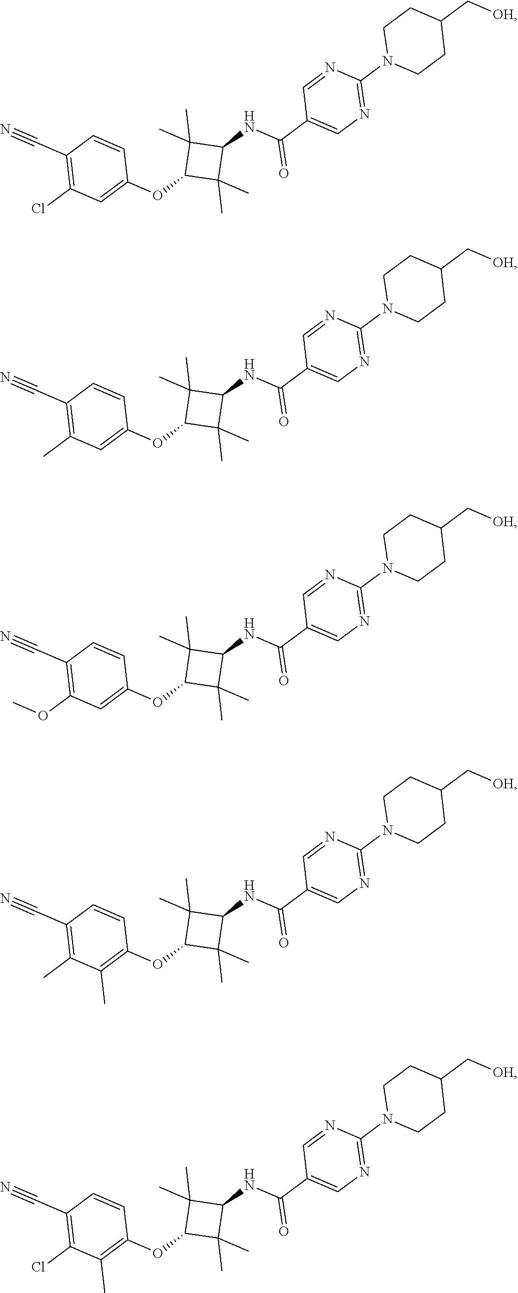 [SMILES: CC1(C)[C@H](NC(=O)c2cnc(N3CCC(CO)CC3)nc2)C(C)(C)[C@H]1Oc1ccc(C#N)c(Cl)c1 ; Cc1cc(O[C@H]2C(C)(C)[C@H](NC(=O)c3cnc(N4CCC(CO)CC4)nc3)C2(C)C)ccc1C#N ; COc1cc(O[C@H]2C(C)(C)[C@H](NC(=O)c3cnc(N4CCC(CO)CC4)nc3)C2(C)C)ccc1C#N ; Cc1c(C#N)ccc(O[C@H]2C(C)(C)[C@H](NC(=O)c3cnc(N4CCC(CO)CC4)nc3)C2(C)C)c1C ; Cc1c(O[C@H]2C(C)(C)[C@H](NC(=O)c3cnc(N4CCC(CO)CC4)nc3)C2(C)C)ccc(C#N)c1Cl]

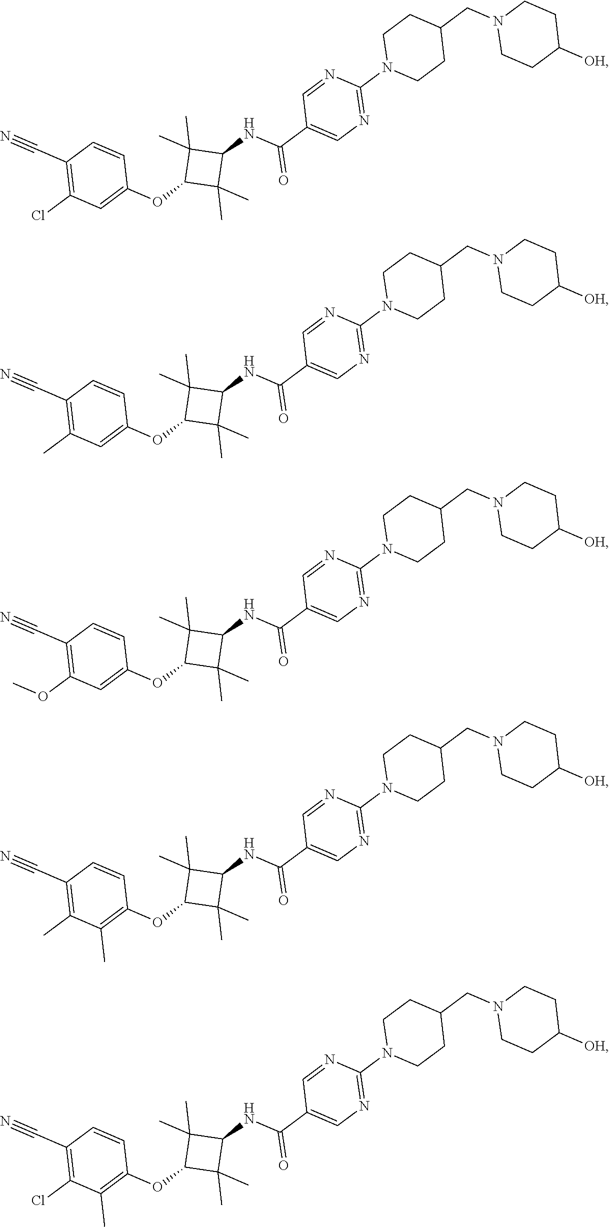 [SMILES: CC1(C)[C@H](NC(=O)c2cnc(N3CCC(CN4CCC(O)CC4)CC3)nc2)C(C)(C)[C@H]1Oc1ccc(C#N)c(Cl)c1 ; Cc1cc(O[C@H]2C(C)(C)[C@H](NC(=O)c3cnc(N4CCC(CN5CCC(O)CC5)CC4)nc3)C2(C)C)ccc1C#N ; COc1cc(O[C@H]2C(C)(C)[C@H](NC(=O)c3cnc(N4CCC(CN5CCC(O)CC5)CC4)nc3)C2(C)C)ccc1C#N ; Cc1c(C#N)ccc(O[C@H]2C(C)(C)[C@H](NC(=O)c3cnc(N4CCC(CN5CCC(O)CC5)CC4)nc3)C2(C)C)c1C ; Cc1c(O[C@H]2C(C)(C)[C@H](NC(=O)c3cnc(N4CCC(CN5CCC(O)CC5)CC4)nc3)C2(C)C)ccc(C#N)c1Cl]

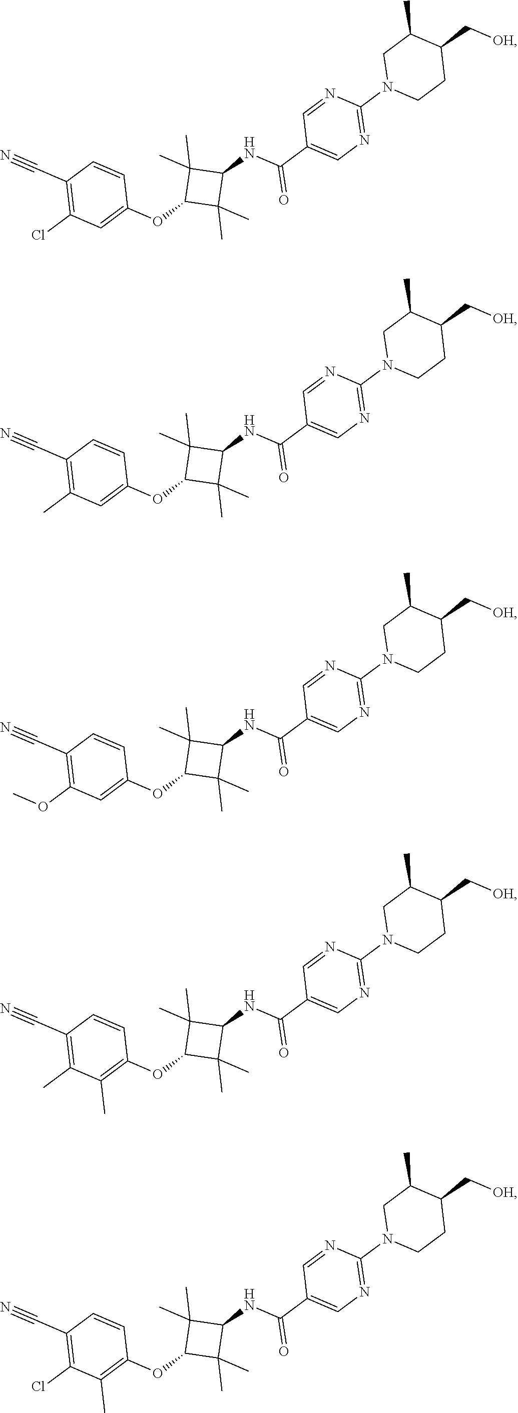 [SMILES: C[C@@H]1CN(c2ncc(C(=O)N[C@H]3C(C)(C)[C@H](Oc4ccc(C#N)c(Cl)c4)C3(C)C)cn2)CC[C@@H]1CO ; Cc1cc(O[C@H]2C(C)(C)[C@H](NC(=O)c3cnc(N4CC[C@H](CO)[C@H](C)C4)nc3)C2(C)C)ccc1C#N ; COc1cc(O[C@H]2C(C)(C)[C@H](NC(=O)c3cnc(N4CC[C@H](CO)[C@H](C)C4)nc3)C2(C)C)ccc1C#N ; Cc1c(C#N)ccc(O[C@H]2C(C)(C)[C@H](NC(=O)c3cnc(N4CC[C@H](CO)[C@H](C)C4)nc3)C2(C)C)c1C ; Cc1c(O[C@H]2C(C)(C)[C@H](NC(=O)c3cnc(N4CC[C@H](CO)[C@H](C)C4)nc3)C2(C)C)ccc(C#N)c1Cl]

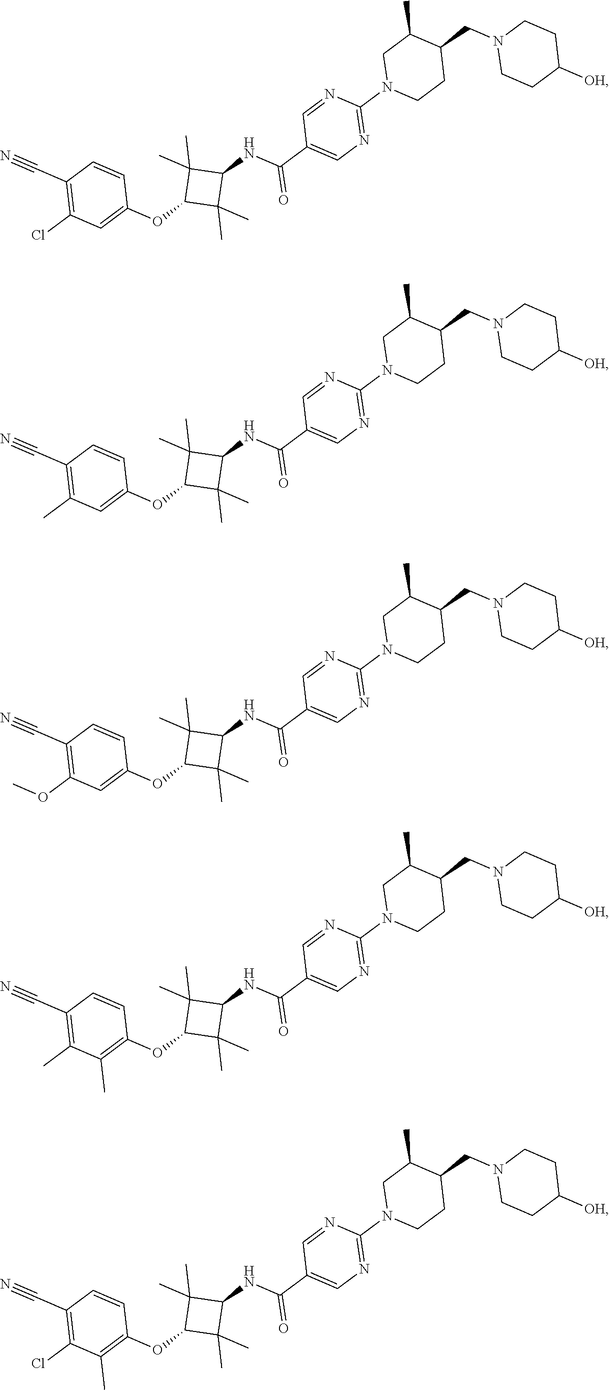 [SMILES: C[C@@H]1CN(c2ncc(C(=O)N[C@H]3C(C)(C)[C@H](Oc4ccc(C#N)c(Cl)c4)C3(C)C)cn2)CC[C@@H]1CN1CCC(O)CC1 ; Cc1cc(O[C@H]2C(C)(C)[C@H](NC(=O)c3cnc(N4CC[C@H](CN5CCC(O)CC5)[C@H](C)C4)nc3)C2(C)C)ccc1C#N ; COc1cc(O[C@H]2C(C)(C)[C@H](NC(=O)c3cnc(N4CC[C@H](CN5CCC(O)CC5)[C@H](C)C4)nc3)C2(C)C)ccc1C#N ; Cc1c(C#N)ccc(O[C@H]2C(C)(C)[C@H](NC(=O)c3cnc(N4CC[C@H](CN5CCC(O)CC5)[C@H](C)C4)nc3)C2(C)C)c1C ; Cc1c(O[C@H]2C(C)(C)[C@H](NC(=O)c3cnc(N4CC[C@H](CN5CCC(O)CC5)[C@H](C)C4)nc3)C2(C)C)ccc(C#N)c1Cl]

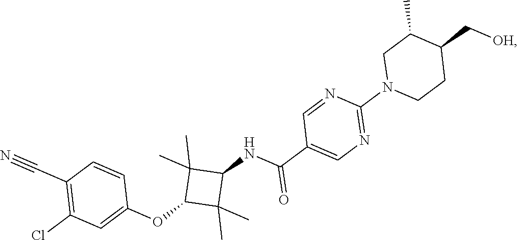 [SMILES: C[C@H]1CN(c2ncc(C(=O)N[C@H]3C(C)(C)[C@H](Oc4ccc(C#N)c(Cl)c4)C3(C)C)cn2)CC[C@@H]1CO]

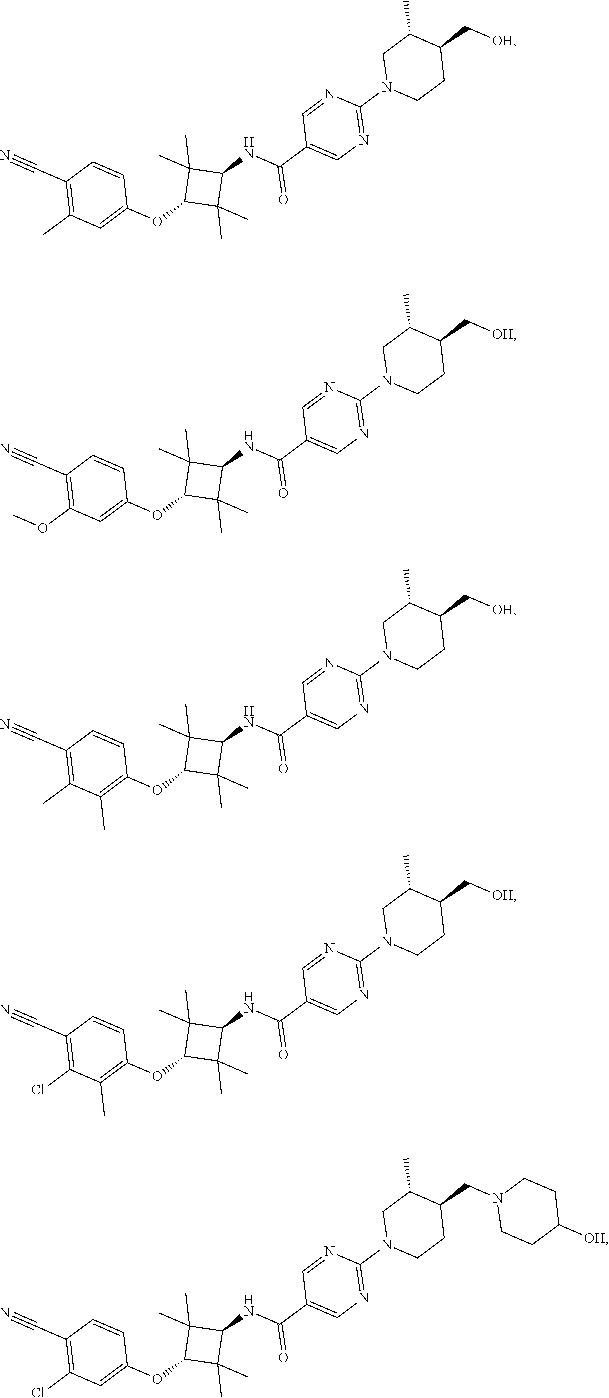 [SMILES: Cc1cc(O[C@H]2C(C)(C)[C@H](NC(=O)c3cnc(N4CC[C@H](CO)[C@@H](C)C4)nc3)C2(C)C)ccc1C#N ; COc1cc(O[C@H]2C(C)(C)[C@H](NC(=O)c3cnc(N4CC[C@H](CO)[C@@H](C)C4)nc3)C2(C)C)ccc1C#N ; Cc1c(C#N)ccc(O[C@H]2C(C)(C)[C@H](NC(=O)c3cnc(N4CC[C@H](CO)[C@@H](C)C4)nc3)C2(C)C)c1C ; Cc1c(O[C@H]2C(C)(C)[C@H](NC(=O)c3cnc(N4CC[C@H](CO)[C@@H](C)C4)nc3)C2(C)C)ccc(C#N)c1Cl ; C[C@H]1CN(c2ncc(C(=O)N[C@H]3C(C)(C)[C@H](Oc4ccc(C#N)c(Cl)c4)C3(C)C)cn2)CC[C@@H]1CN1CCC(O)CC1]

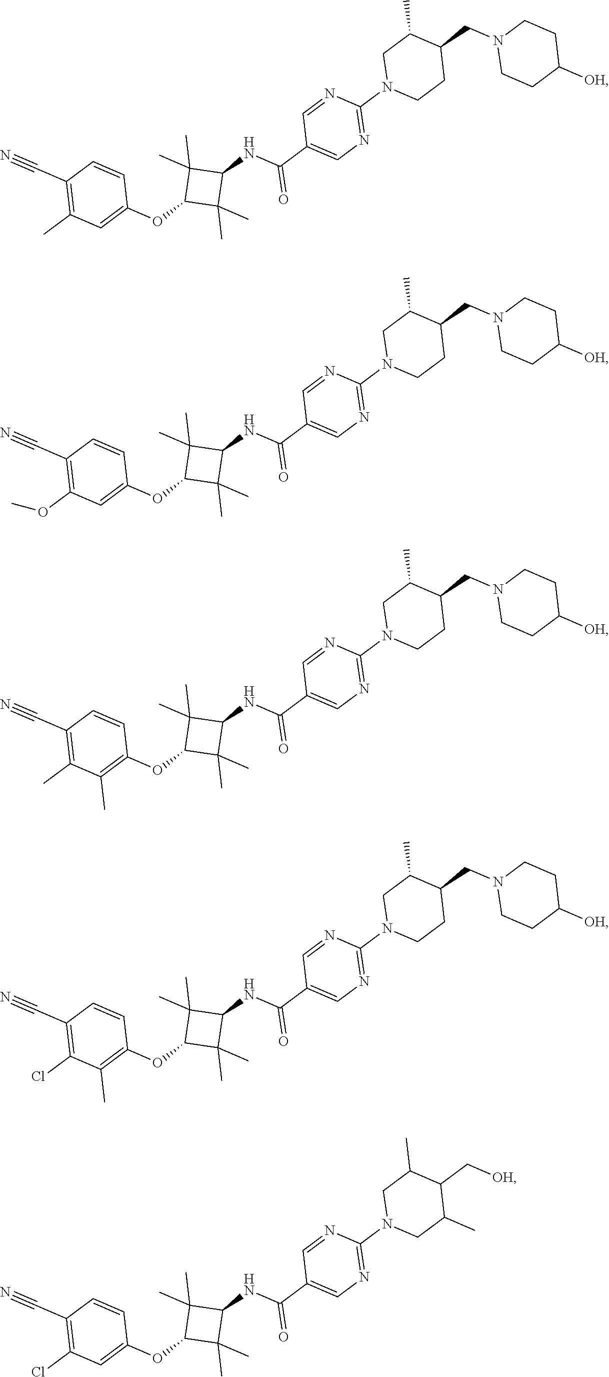 [SMILES: Cc1cc(O[C@H]2C(C)(C)[C@H](NC(=O)c3cnc(N4CC[C@H](CN5CCC(O)CC5)[C@@H](C)C4)nc3)C2(C)C)ccc1C#N ; COc1cc(O[C@H]2C(C)(C)[C@H](NC(=O)c3cnc(N4CC[C@H](CN5CCC(O)CC5)[C@@H](C)C4)nc3)C2(C)C)ccc1C#N ; Cc1c(C#N)ccc(O[C@H]2C(C)(C)[C@H](NC(=O)c3cnc(N4CC[C@H](CN5CCC(O)CC5)[C@@H](C)C4)nc3)C2(C)C)c1C ; Cc1c(O[C@H]2C(C)(C)[C@H](NC(=O)c3cnc(N4CC[C@H](CN5CCC(O)CC5)[C@@H](C)C4)nc3)C2(C)C)ccc(C#N)c1Cl ; CC1CN(c2ncc(C(=O)N[C@H]3C(C)(C)[C@H](Oc4ccc(C#N)c(Cl)c4)C3(C)C)cn2)CC(C)C1CO]

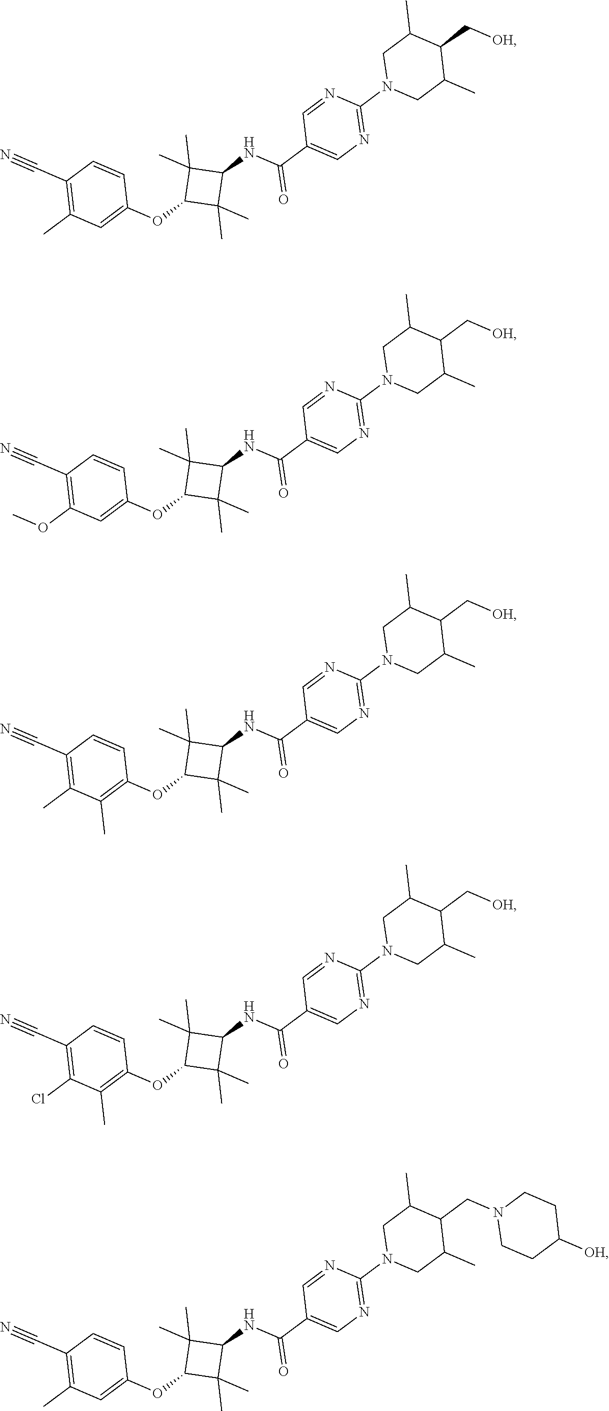

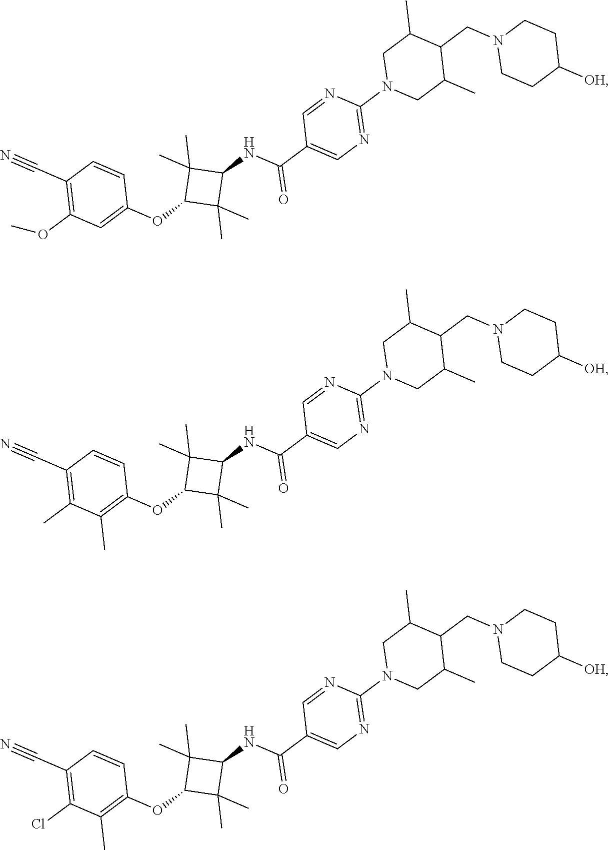 [SMILES: COc1cc(O[C@H]2C(C)(C)[C@H](NC(=O)c3cnc(N4CC(C)C(CN5CCC(O)CC5)C(C)C4)nc3)C2(C)C)ccc1C#N ; Cc1c(C#N)ccc(O[C@H]2C(C)(C)[C@H](NC(=O)c3cnc(N4CC(C)C(CN5CCC(O)CC5)C(C)C4)nc3)C2(C)C)c1C ; Cc1c(O[C@H]2C(C)(C)[C@H](NC(=O)c3cnc(N4CC(C)C(CN5CCC(O)CC5)C(C)C4)nc3)C2(C)C)ccc(C#N)c1Cl]

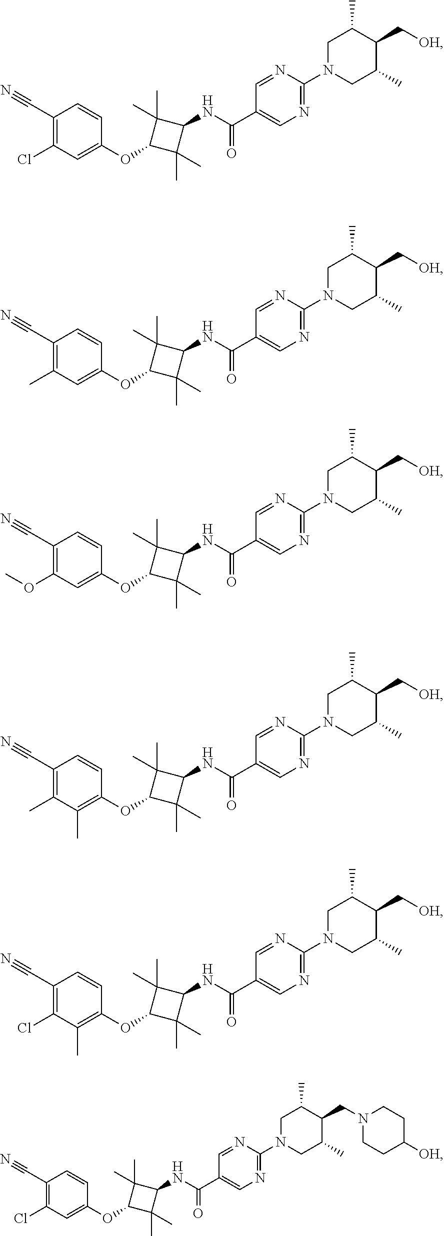

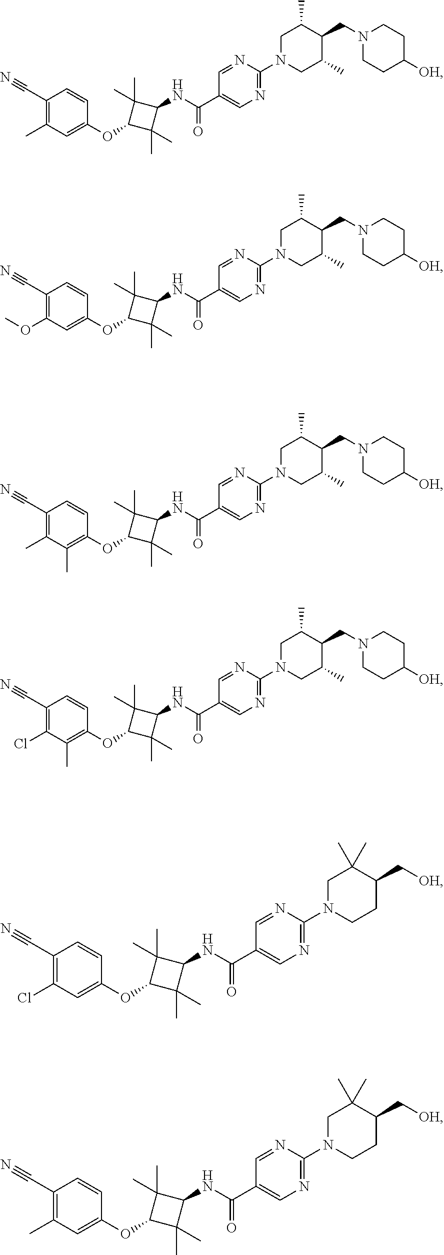

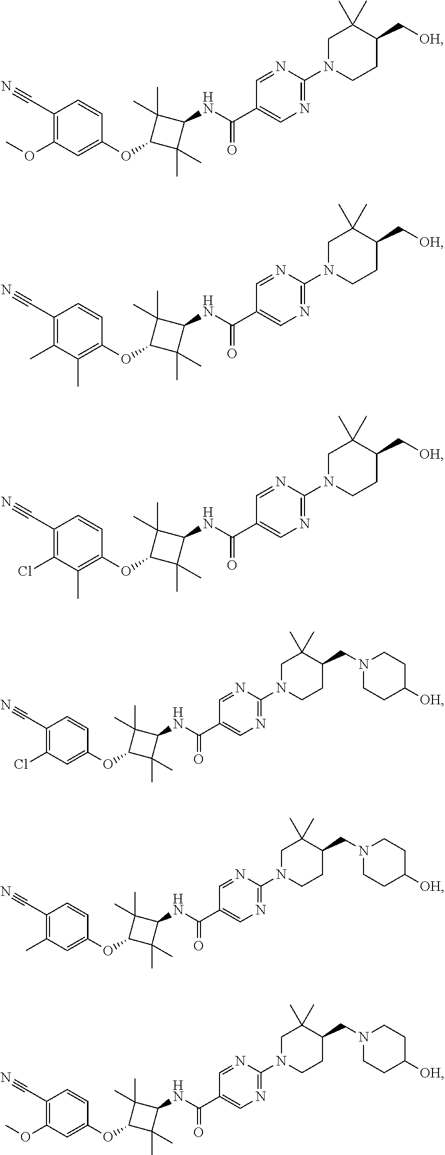

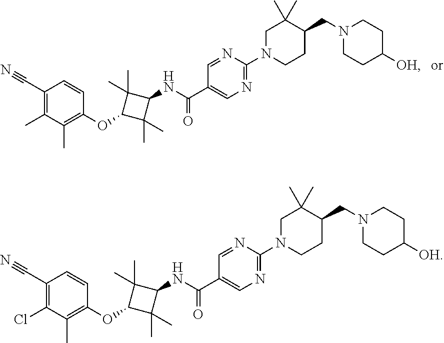 [SMILES: Cc1c(C#N)ccc(O[C@H]2C(C)(C)[C@H](NC(=O)c3cnc(N4CC[C@H](CN5CCC(O)CC5)C(C)(C)C4)nc3)C2(C)C)c1C ; Cc1c(O[C@H]2C(C)(C)[C@H](NC(=O)c3cnc(N4CC[C@H](CN5CCC(O)CC5)C(C)(C)C4)nc3)C2(C)C)ccc(C#N)c1Cl]

In some embodiments, the compound is a compound of Table 1, or a pharmaceutically acceptable salt or solvate thereof:

TABLE 1

| Com- | |
|---|---|
| pound | Structure |
| 1 | 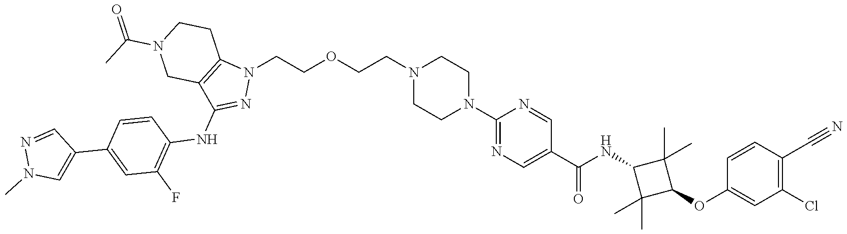 [SMILES: CC(=O)N1CCc2c(c(Nc3ccc(-c4cnn(C)c4)cc3F)nn2CCOCCN2CCN(c3ncc(C(=O)N[C@H]4C(C)(C)[C@H](Oc5ccc(C#N)c(Cl)c5)C4(C)C)cn3)CC2)C1] |
| 2 | 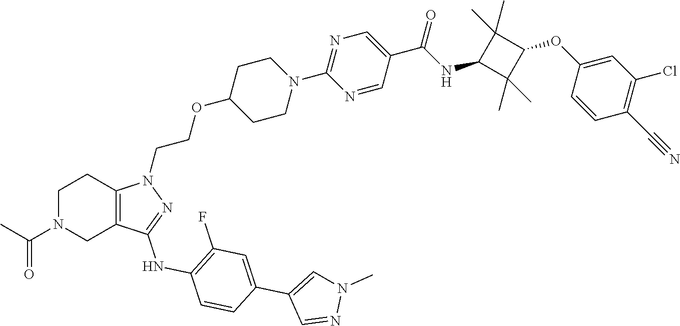 [SMILES: CC(=O)N1CCc2c(c(Nc3ccc(-c4cnn(C)c4)cc3F)nn2CCOC2CCN(c3ncc(C(=O)N[C@H]4C(C)(C)[C@H](Oc5ccc(C#N)c(Cl)c5)C4(C)C)cn3)CC2)C1] |
| 3 | 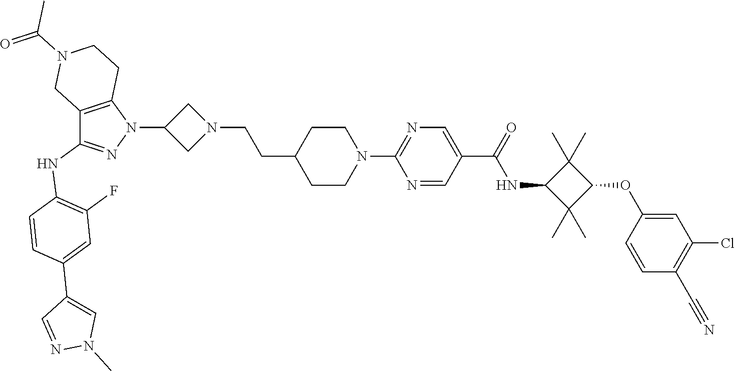 [SMILES: CC(=O)N1CCc2c(c(Nc3ccc(-c4cnn(C)c4)cc3F)nn2C2CN(CCC3CCN(c4ncc(C(=O)N[C@H]5C(C)(C)[C@H](Oc6ccc(C#N)c(Cl)c6)C5(C)C)cn4)CC3)C2)C1] |
| 4 | 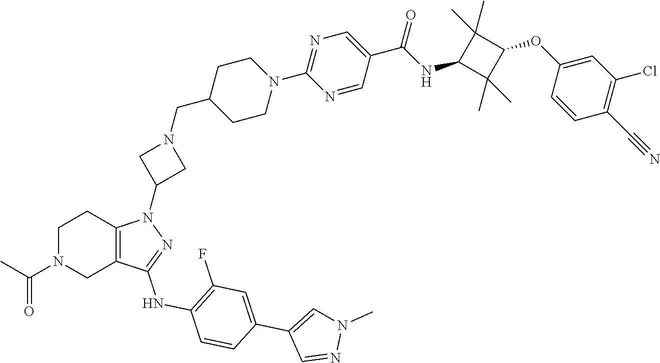 [SMILES: CC(=O)N1CCc2c(c(Nc3ccc(-c4cnn(C)c4)cc3F)nn2C2CN(CC3CCN(c4ncc(C(=O)N[C@H]5C(C)(C)[C@H](Oc6ccc(C#N)c(Cl)c6)C5(C)C)cn4)CC3)C2)C1] |
| 5 | 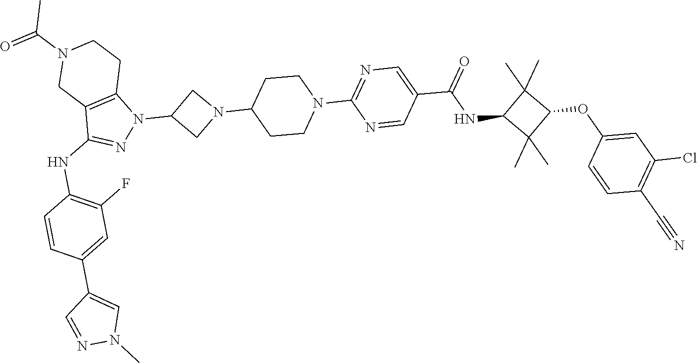 [SMILES: CC(=O)N1CCc2c(c(Nc3ccc(-c4cnn(C)c4)cc3F)nn2C2CN(C3CCN(c4ncc(C(=O)N[C@H]5C(C)(C)[C@H](Oc6ccc(C#N)c(Cl)c6)C5(C)C)cn4)CC3)C2)C1] |
| 6 | 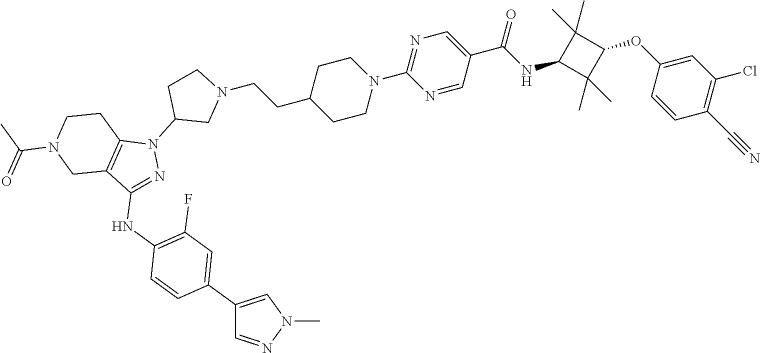 [SMILES: CC(=O)N1CCc2c(c(Nc3ccc(-c4cnn(C)c4)cc3F)nn2C2CCN(CCC3CCN(c4ncc(C(=O)N[C@H]5C(C)(C)[C@H](Oc6ccc(C#N)c(Cl)c6)C5(C)C)cn4)CC3)C2)C1] |
| 7 | 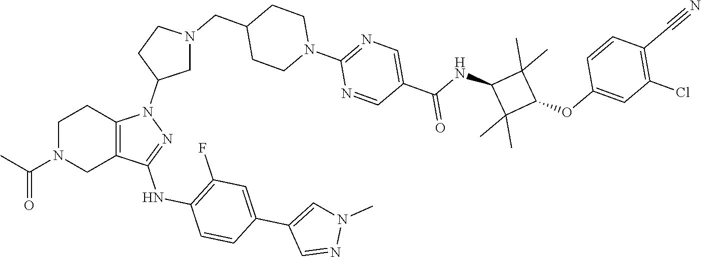 [SMILES: CC(=O)N1CCc2c(c(Nc3ccc(-c4cnn(C)c4)cc3F)nn2C2CCN(CC3CCN(c4ncc(C(=O)N[C@H]5C(C)(C)[C@H](Oc6ccc(C#N)c(Cl)c6)C5(C)C)cn4)CC3)C2)C1] |
| 8 | 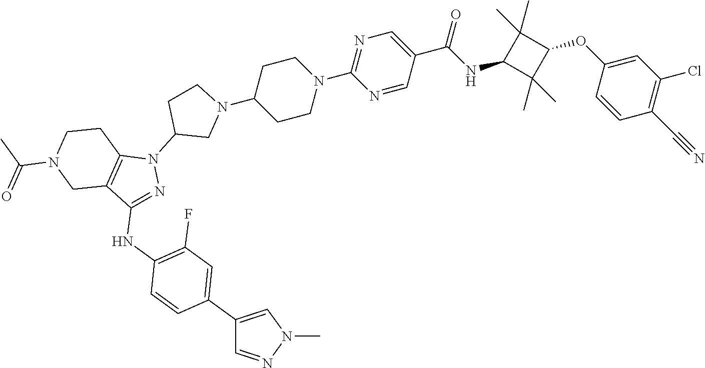 [SMILES: CC(=O)N1CCc2c(c(Nc3ccc(-c4cnn(C)c4)cc3F)nn2C2CCN(C3CCN(c4ncc(C(=O)N[C@H]5C(C)(C)[C@H](Oc6ccc(C#N)c(Cl)c6)C5(C)C)cn4)CC3)C2)C1] |
| 9 | 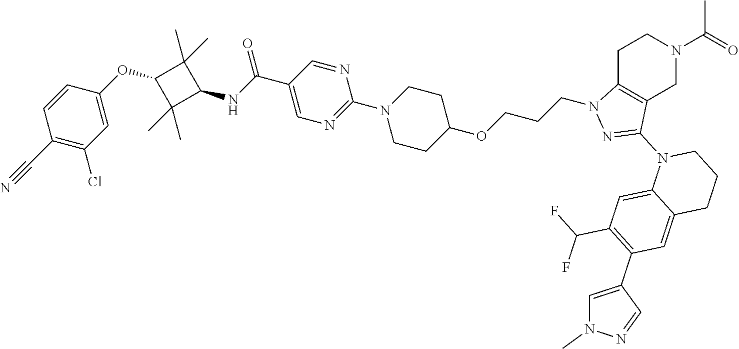 [SMILES: CC(=O)N1CCc2c(c(N3CCCc4cc(-c5cnn(C)c5)c(C(F)F)cc43)nn2CCCOC2CCN(c3ncc(C(=O)N[C@H]4C(C)(C)[C@H](Oc5ccc(C#N)c(Cl)c5)C4(C)C)cn3)CC2)C1] |
| 10 | 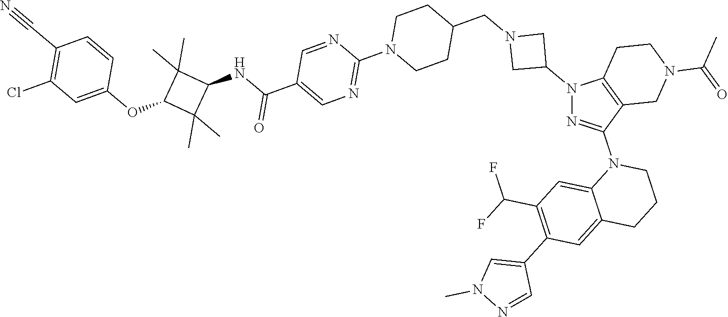 [SMILES: CC(=O)N1CCc2c(c(N3CCCc4cc(-c5cnn(C)c5)c(C(F)F)cc43)nn2C2CN(CC3CCN(c4ncc(C(=O)N[C@H]5C(C)(C)[C@H](Oc6ccc(C#N)c(Cl)c6)C5(C)C)cn4)CC3)C2)C1] |
| 11 | 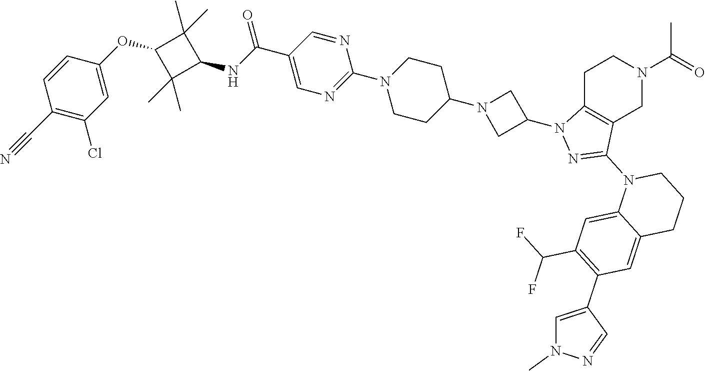 [SMILES: CC(=O)N1CCc2c(c(N3CCCc4cc(-c5cnn(C)c5)c(C(F)F)cc43)nn2C2CN(C3CCN(c4ncc(C(=O)N[C@H]5C(C)(C)[C@H](Oc6ccc(C#N)c(Cl)c6)C5(C)C)cn4)CC3)C2)C1] |
| 12 | 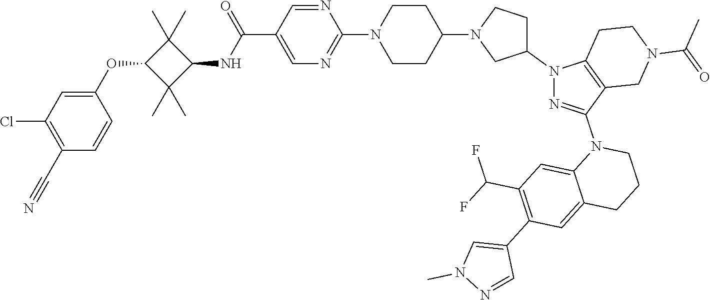 [SMILES: CC(=O)N1CCc2c(c(N3CCCc4cc(-c5cnn(C)c5)c(C(F)F)cc43)nn2C2CCN(C3CCN(c4ncc(C(=O)N[C@H]5C(C)(C)[C@H](Oc6ccc(C#N)c(Cl)c6)C5(C)C)cn4)CC3)C2)C1] |
| 13 | 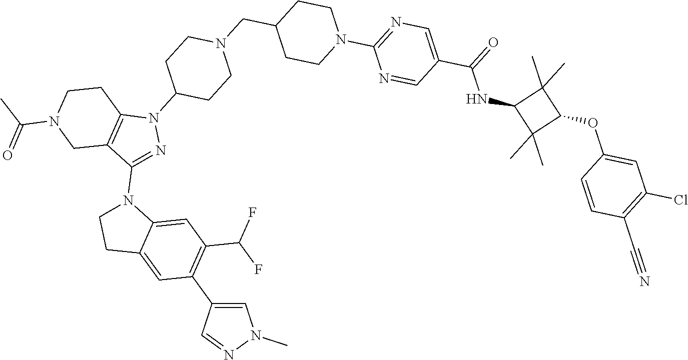 [SMILES: CC(=O)N1CCc2c(c(N3CCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 14 | 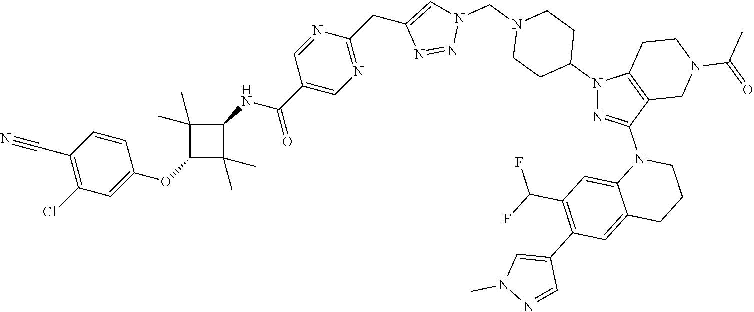 [SMILES: CC(=O)N1CCc2c(c(N3CCCc4cc(-c5cnn(C)c5)c(C(F)F)cc43)nn2C2CCN(Cn3cc(Cc4ncc(C(=O)N[C@H]5C(C)(C)[C@H](Oc6ccc(C#N)c(Cl)c6)C5(C)C)cn4)nn3)CC2)C1] |
| 15 | 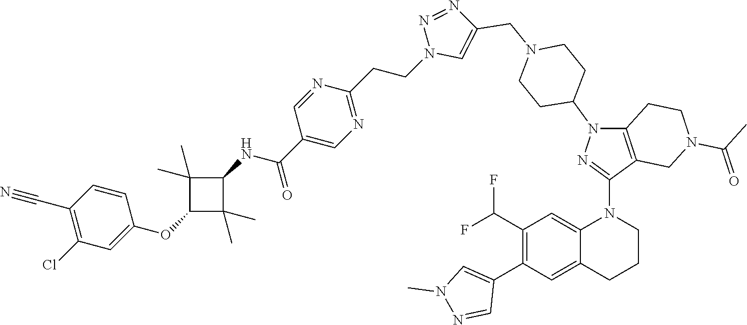 [SMILES: CC(=O)N1CCc2c(c(N3CCCc4cc(-c5cnn(C)c5)c(C(F)F)cc43)nn2C2CCN(Cc3cn(CCc4ncc(C(=O)N[C@H]5C(C)(C)[C@H](Oc6ccc(C#N)c(Cl)c6)C5(C)C)cn4)nn3)CC2)C1] |
| 16 | 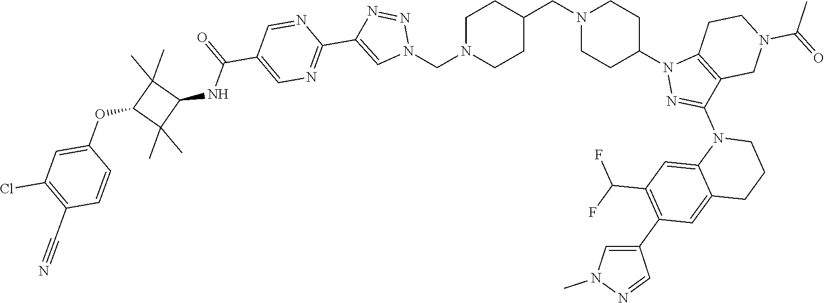 [SMILES: CC(=O)N1CCc2c(c(N3CCCc4cc(-c5cnn(C)c5)c(C(F)F)cc43)nn2C2CCN(CC3CCN(Cn4cc(-c5ncc(C(=O)N[C@H]6C(C)(C)[C@H](Oc7ccc(C#N)c(Cl)c7)C6(C)C)cn5)nn4)CC3)CC2)C1] |
| 17 | 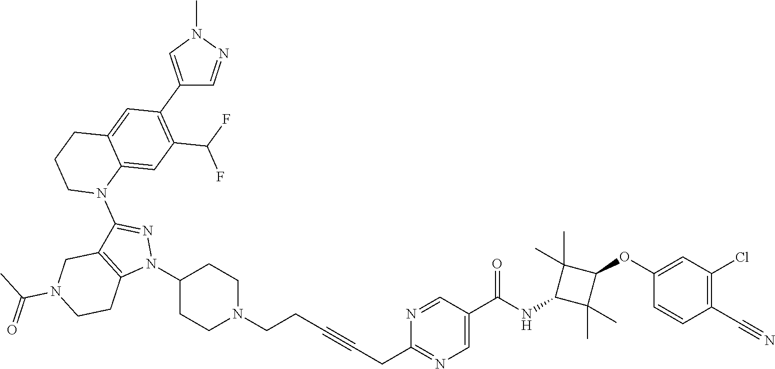 [SMILES: CC(=O)N1CCc2c(c(N3CCCc4cc(-c5cnn(C)c5)c(C(F)F)cc43)nn2C2CCN(CCC#CCc3ncc(C(=O)N[C@H]4C(C)(C)[C@H](Oc5ccc(C#N)c(Cl)c5)C4(C)C)cn3)CC2)C1] |
| 18 | 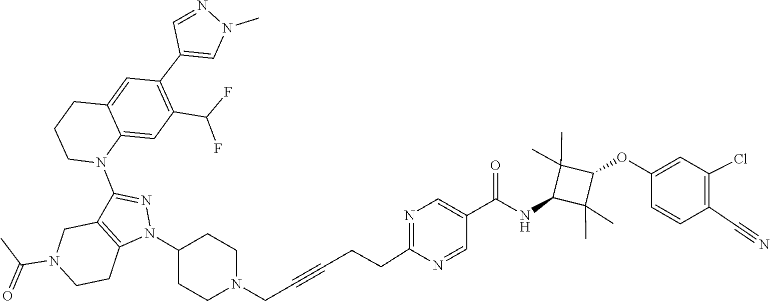 [SMILES: CC(=O)N1CCc2c(c(N3CCCc4cc(-c5cnn(C)c5)c(C(F)F)cc43)nn2C2CCN(CC#CCCc3ncc(C(=O)N[C@H]4C(C)(C)[C@H](Oc5ccc(C#N)c(Cl)c5)C4(C)C)cn3)CC2)C1] |
| 19 | 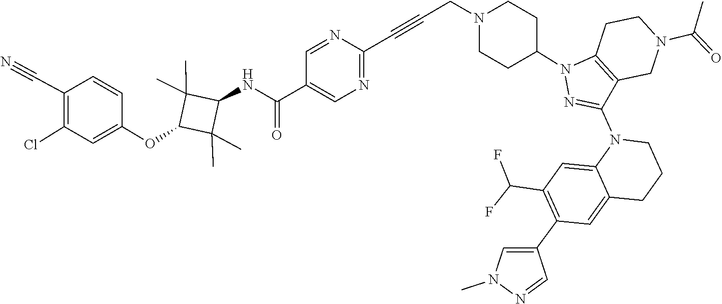 [SMILES: CC(=O)N1CCc2c(c(N3CCCc4cc(-c5cnn(C)c5)c(C(F)F)cc43)nn2C2CCN(CC#Cc3ncc(C(=O)N[C@H]4C(C)(C)[C@H](Oc5ccc(C#N)c(Cl)c5)C4(C)C)cn3)CC2)C1] |
| 20 | 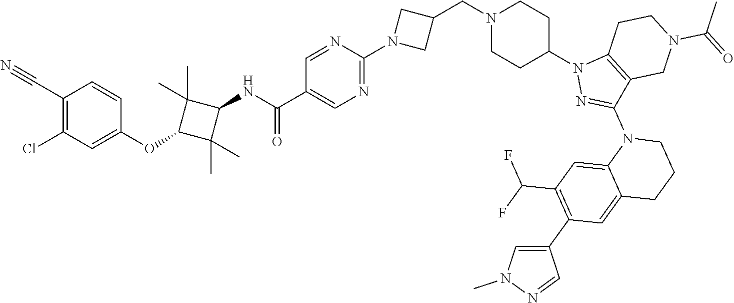 [SMILES: CC(=O)N1CCc2c(c(N3CCCc4cc(-c5cnn(C)c5)c(C(F)F)cc43)nn2C2CCN(CC3CN(c4ncc(C(=O)N[C@H]5C(C)(C)[C@H](Oc6ccc(C#N)c(Cl)c6)C5(C)C)cn4)C3)CC2)C1] |
| 21 | 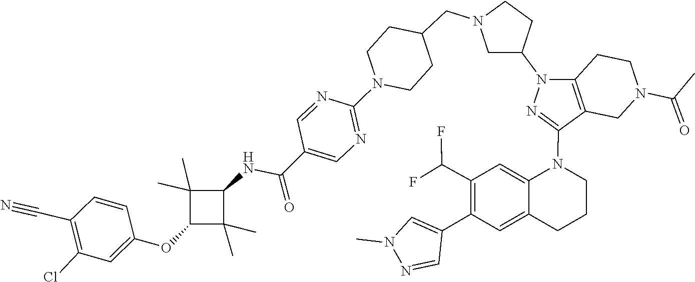 [SMILES: CC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)C2)C1] |
| 22 | 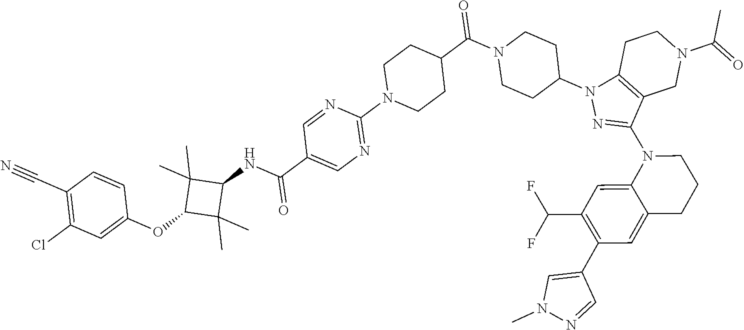 [SMILES: CC(=O)N1CCc2c(c(N3CCCc4cc(-c5cnn(C)c5)c(C(F)F)cc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1] |
| 23 | 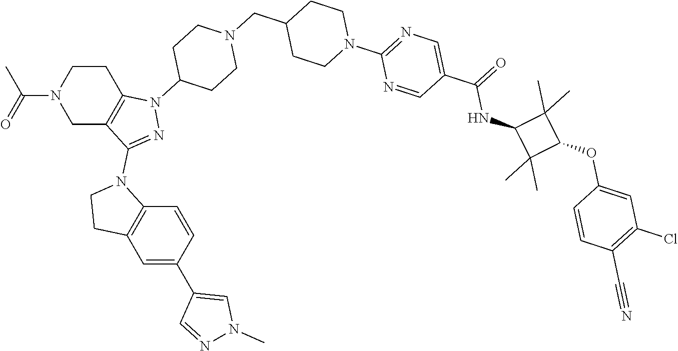 [SMILES: CC(=O)N1CCc2c(c(N3CCc4cc(-c5cnn(C)c5)ccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 24 | 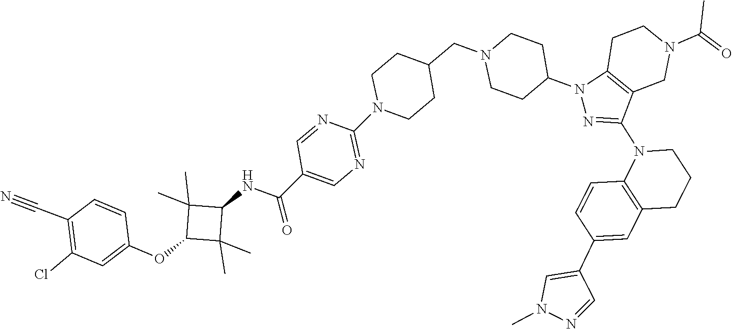 [SMILES: CC(=O)N1CCc2c(c(N3CCCc4cc(-c5cnn(C)c5)ccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 25 | 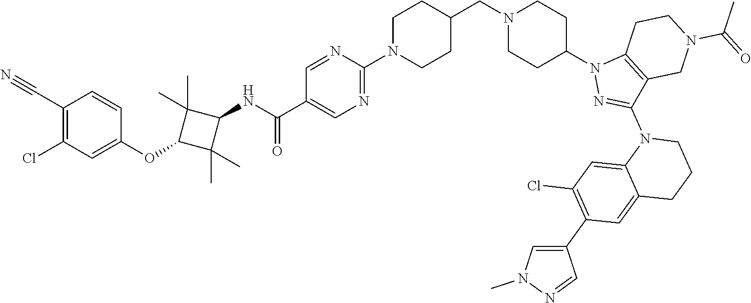 [SMILES: CC(=O)N1CCc2c(c(N3CCCc4cc(-c5cnn(C)c5)c(Cl)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 26 | 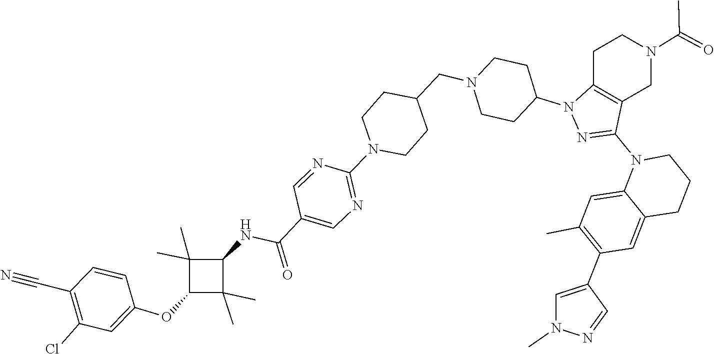 [SMILES: CC(=O)N1CCc2c(c(N3CCCc4cc(-c5cnn(C)c5)c(C)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 27 | 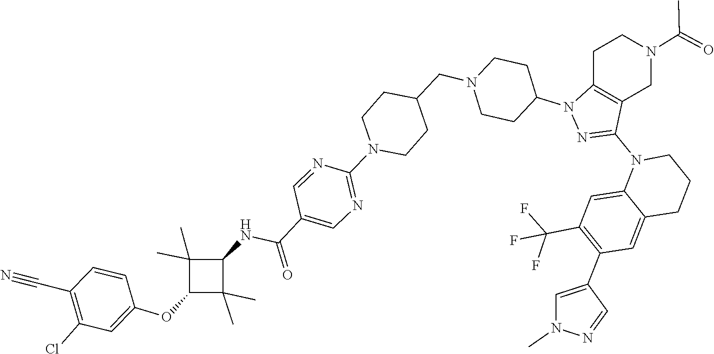 [SMILES: CC(=O)N1CCc2c(c(N3CCCc4cc(-c5cnn(C)c5)c(C(F)(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 28 | 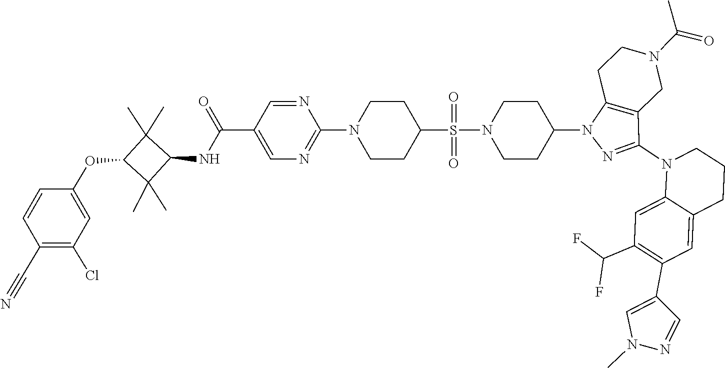 [SMILES: CC(=O)N1CCc2c(c(N3CCCc4cc(-c5cnn(C)c5)c(C(F)F)cc43)nn2C2CCN(S(=O)(=O)C3CCN(c4ncc(C(=O)N[C@H]5C(C)(C)[C@H](Oc6ccc(C#N)c(Cl)c6)C5(C)C)cn4)CC3)CC2)C1] |
| 29 | 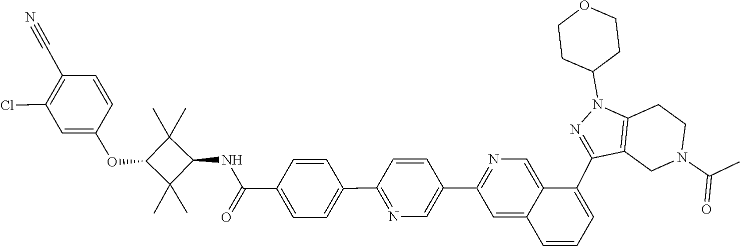 [SMILES: CC(=O)N1CCc2c(c(-c3cccc4cc(-c5ccc(-c6ccc(C(=O)N[C@H]7C(C)(C)[C@H](Oc8ccc(C#N)c(Cl)c8)C7(C)C)cc6)nc5)ncc34)nn2C2CCOCC2)C1] |
| 30 | 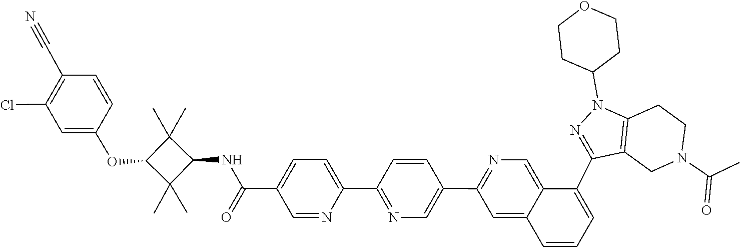 [SMILES: CC(=O)N1CCc2c(c(-c3cccc4cc(-c5ccc(-c6ccc(C(=O)N[C@H]7C(C)(C)[C@H](Oc8ccc(C#N)c(Cl)c8)C7(C)C)cn6)nc5)ncc34)nn2C2CCOCC2)C1] |
| 31 | 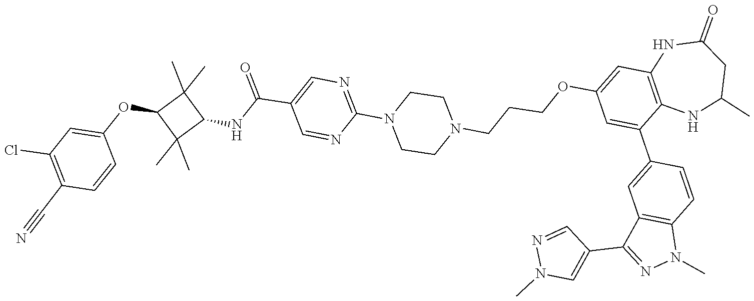 [SMILES: CC1CC(=O)Nc2cc(OCCCN3CCN(c4ncc(C(=O)N[C@H]5C(C)(C)[C@H](Oc6ccc(C#N)c(Cl)c6)C5(C)C)cn4)CC3)cc(-c3ccc4c(c3)c(-c3cnn(C)c3)nn4C)c2N1] |
| 32 | 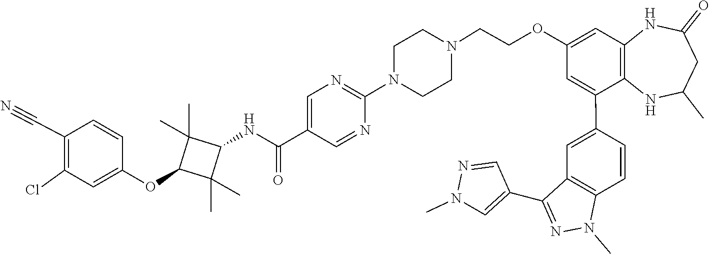 [SMILES: CC1CC(=O)Nc2cc(OCCN3CCN(c4ncc(C(=O)N[C@H]5C(C)(C)[C@H](Oc6ccc(C#N)c(Cl)c6)C5(C)C)cn4)CC3)cc(-c3ccc4c(c3)c(-c3cnn(C)c3)nn4C)c2N1] |
| 33 | 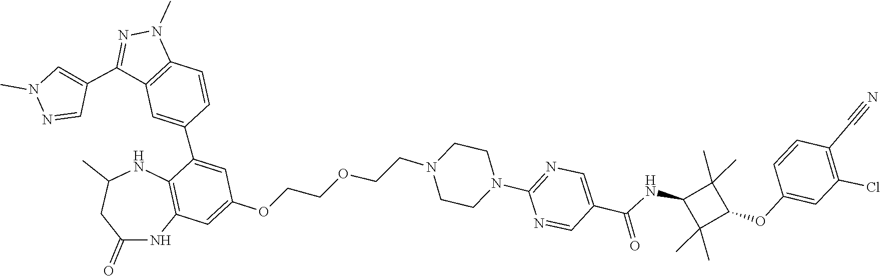 [SMILES: CC1CC(=O)Nc2cc(OCCOCCN3CCN(c4ncc(C(=O)N[C@H]5C(C)(C)[C@H](Oc6ccc(C#N)c(Cl)c6)C5(C)C)cn4)CC3)cc(-c3ccc4c(c3)c(-c3cnn(C)c3)nn4C)c2N1] |
| 34 | 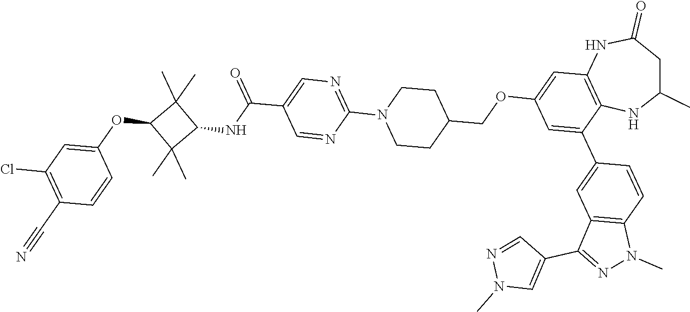 [SMILES: CC1CC(=O)Nc2cc(OCC3CCN(c4ncc(C(=O)N[C@H]5C(C)(C)[C@H](Oc6ccc(C#N)c(Cl)c6)C5(C)C)cn4)CC3)cc(-c3ccc4c(c3)c(-c3cnn(C)c3)nn4C)c2N1] |
| 35 | 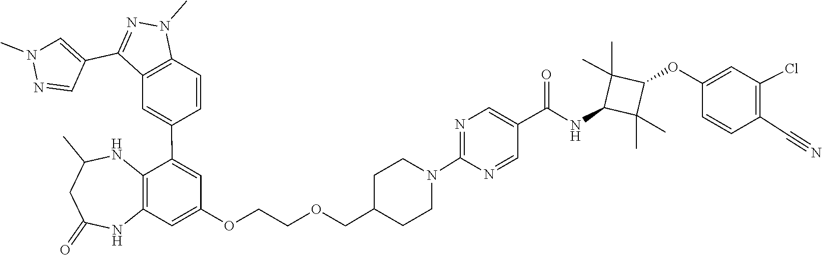 [SMILES: CC1CC(=O)Nc2cc(OCCOCC3CCN(c4ncc(C(=O)N[C@H]5C(C)(C)[C@H](Oc6ccc(C#N)c(Cl)c6)C5(C)C)cn4)CC3)cc(-c3ccc4c(c3)c(-c3cnn(C)c3)nn4C)c2N1] |
| 36 | 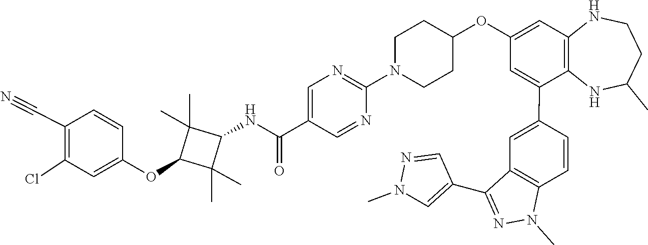 [SMILES: CC1CCNc2cc(OC3CCN(c4ncc(C(=O)N[C@H]5C(C)(C)[C@H](Oc6ccc(C#N)c(Cl)c6)C5(C)C)cn4)CC3)cc(-c3ccc4c(c3)c(-c3cnn(C)c3)nn4C)c2N1] |
| 37 | 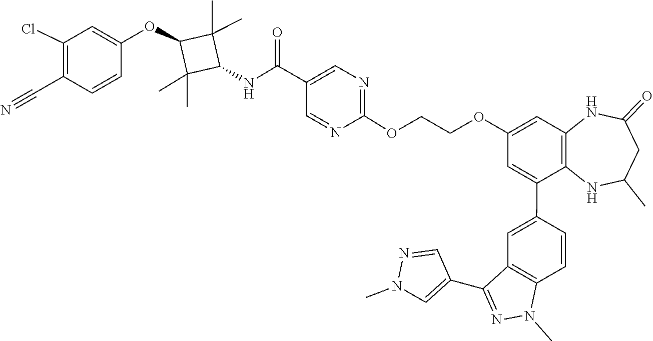 [SMILES: CC1CC(=O)Nc2cc(OCCOc3ncc(C(=O)N[C@H]4C(C)(C)[C@H](Oc5ccc(C#N)c(Cl)c5)C4(C)C)cn3)cc(-c3ccc4c(c3)c(-c3cnn(C)c3)nn4C)c2N1] |
| 38 | 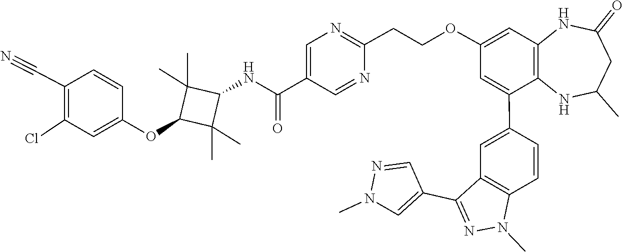 [SMILES: CC1CC(=O)Nc2cc(OCCc3ncc(C(=O)N[C@H]4C(C)(C)[C@H](Oc5ccc(C#N)c(Cl)c5)C4(C)C)cn3)cc(-c3ccc4c(c3)c(-c3cnn(C)c3)nn4C)c2N1] |
| 39 | 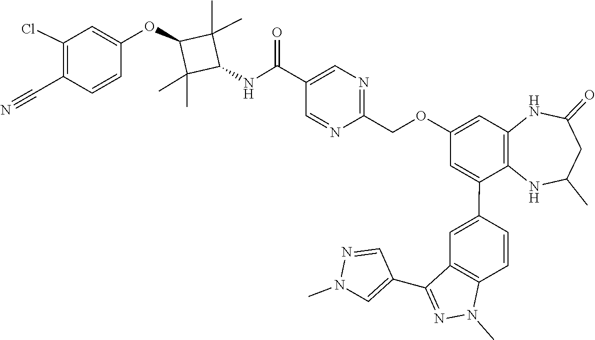 [SMILES: CC1CC(=O)Nc2cc(OCc3ncc(C(=O)N[C@H]4C(C)(C)[C@H](Oc5ccc(C#N)c(Cl)c5)C4(C)C)cn3)cc(-c3ccc4c(c3)c(-c3cnn(C)c3)nn4C)c2N1] |
| 40 | 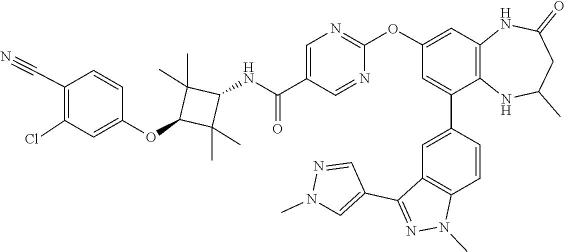 [SMILES: CC1CC(=O)Nc2cc(Oc3ncc(C(=O)N[C@H]4C(C)(C)[C@H](Oc5ccc(C#N)c(Cl)c5)C4(C)C)cn3)cc(-c3ccc4c(c3)c(-c3cnn(C)c3)nn4C)c2N1] |
| 41 | 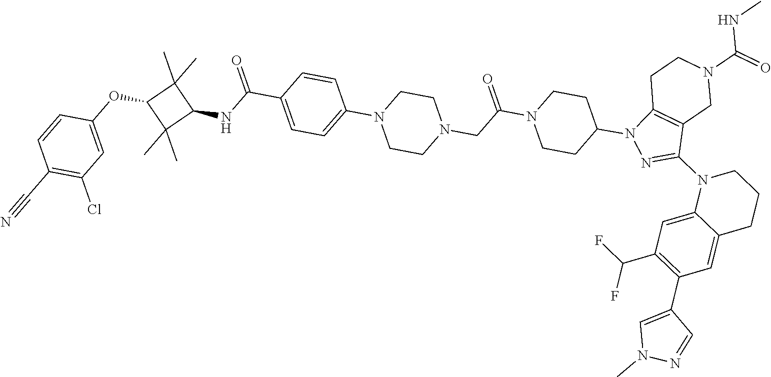 [SMILES: CNC(=O)N1CCc2c(c(N3CCCc4cc(-c5cnn(C)c5)c(C(F)F)cc43)nn2C2CCN(C(=O)CN3CCN(c4ccc(C(=O)N[C@H]5C(C)(C)[C@H](Oc6ccc(C#N)c(Cl)c6)C5(C)C)cc4)CC3)CC2)C1] |
| 42 | 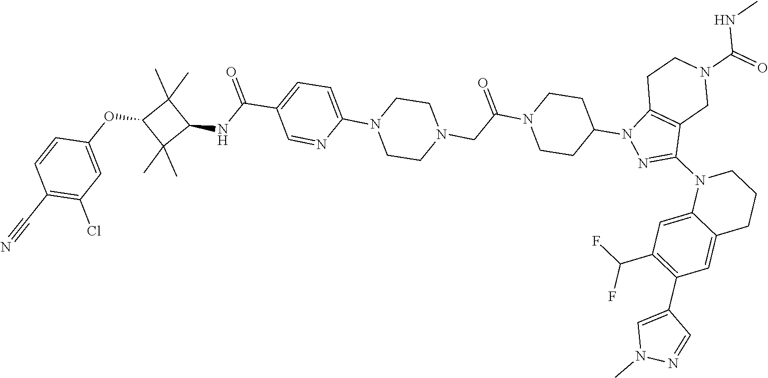 [SMILES: CNC(=O)N1CCc2c(c(N3CCCc4cc(-c5cnn(C)c5)c(C(F)F)cc43)nn2C2CCN(C(=O)CN3CCN(c4ccc(C(=O)N[C@H]5C(C)(C)[C@H](Oc6ccc(C#N)c(Cl)c6)C5(C)C)cn4)CC3)CC2)C1] |
| 43 | 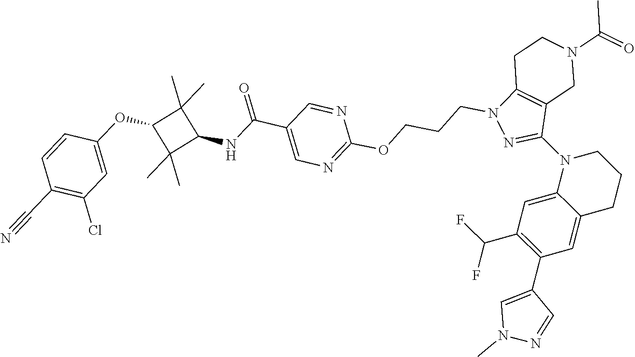 [SMILES: CC(=O)N1CCc2c(c(N3CCCc4cc(-c5cnn(C)c5)c(C(F)F)cc43)nn2CCCOc2ncc(C(=O)N[C@H]3C(C)(C)[C@H](Oc4ccc(C#N)c(Cl)c4)C3(C)C)cn2)C1] |
| 44 | 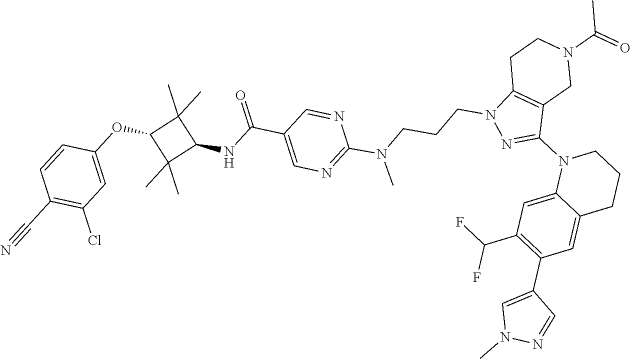 [SMILES: CC(=O)N1CCc2c(c(N3CCCc4cc(-c5cnn(C)c5)c(C(F)F)cc43)nn2CCCN(C)c2ncc(C(=O)N[C@H]3C(C)(C)[C@H](Oc4ccc(C#N)c(Cl)c4)C3(C)C)cn2)C1] |
| 45 | 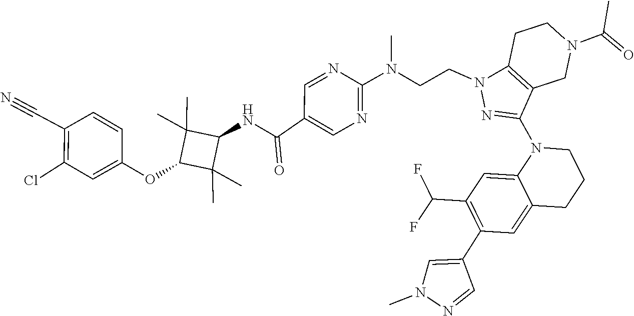 [SMILES: CC(=O)N1CCc2c(c(N3CCCc4cc(-c5cnn(C)c5)c(C(F)F)cc43)nn2CCN(C)c2ncc(C(=O)N[C@H]3C(C)(C)[C@H](Oc4ccc(C#N)c(Cl)c4)C3(C)C)cn2)C1] |
| 46 | 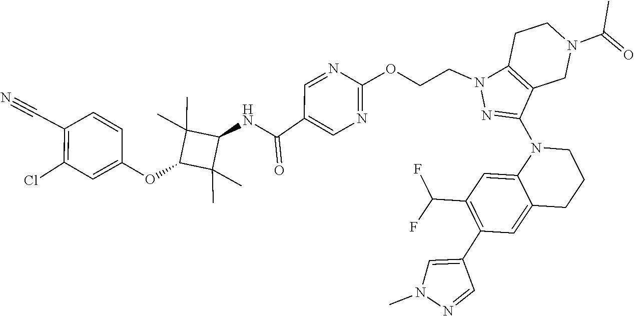 [SMILES: CC(=O)N1CCc2c(c(N3CCCc4cc(-c5cnn(C)c5)c(C(F)F)cc43)nn2CCOc2ncc(C(=O)N[C@H]3C(C)(C)[C@H](Oc4ccc(C#N)c(Cl)c4)C3(C)C)cn2)C1] |
| 47 | 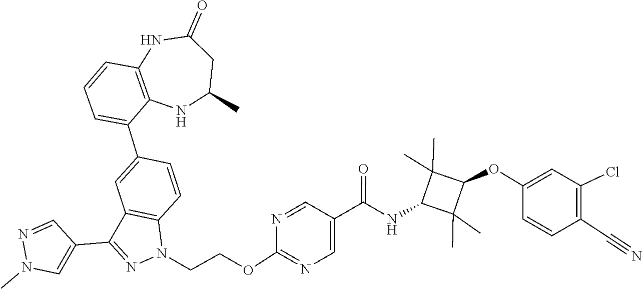 [SMILES: C[C@@H]1CC(=O)Nc2cccc(-c3ccc4c(c3)c(-c3cnn(C)c3)nn4CCOc3ncc(C(=O)N[C@H]4C(C)(C)[C@H](Oc5ccc(C#N)c(Cl)c5)C4(C)C)cn3)c2N1] |
| 48 | 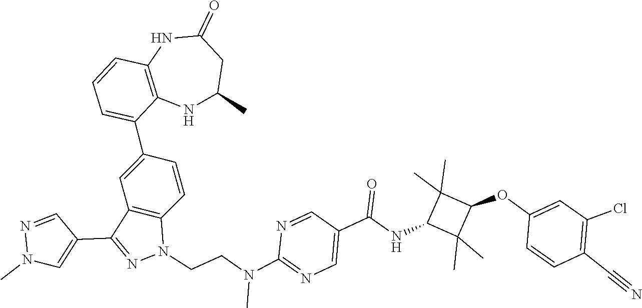 [SMILES: C[C@@H]1CC(=O)Nc2cccc(-c3ccc4c(c3)c(-c3cnn(C)c3)nn4CCN(C)c3ncc(C(=O)N[C@H]4C(C)(C)[C@H](Oc5ccc(C#N)c(Cl)c5)C4(C)C)cn3)c2N1] |
| 49 | 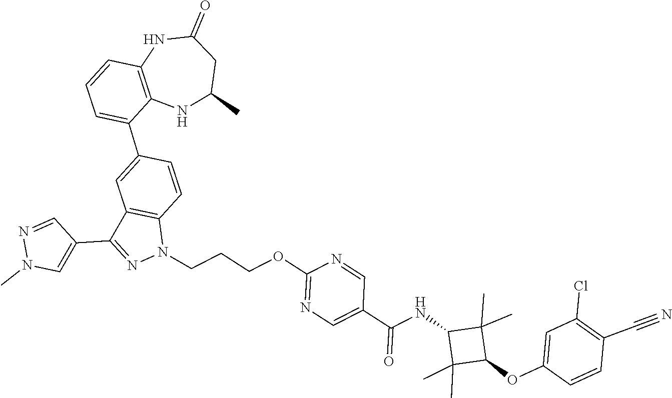 [SMILES: C[C@@H]1CC(=O)Nc2cccc(-c3ccc4c(c3)c(-c3cnn(C)c3)nn4CCCOc3ncc(C(=O)N[C@H]4C(C)(C)[C@H](Oc5ccc(C#N)c(Cl)c5)C4(C)C)cn3)c2N1] |
| 50 | 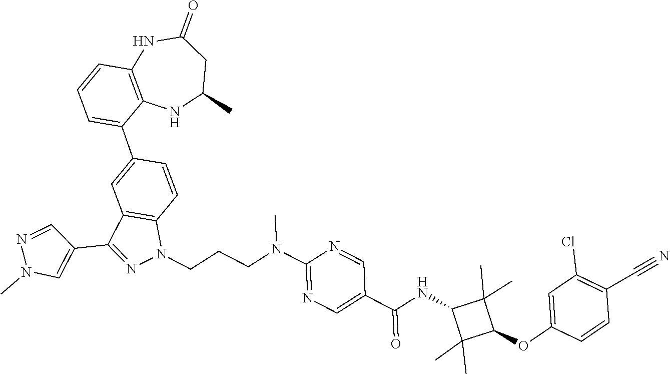 [SMILES: C[C@@H]1CC(=O)Nc2cccc(-c3ccc4c(c3)c(-c3cnn(C)c3)nn4CCCN(C)c3ncc(C(=O)N[C@H]4C(C)(C)[C@H](Oc5ccc(C#N)c(Cl)c5)C4(C)C)cn3)c2N1] |
| 51 | 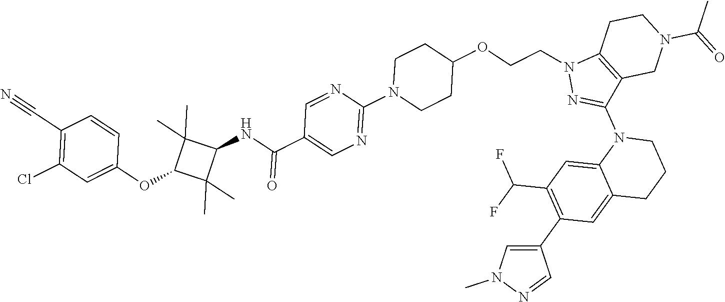 [SMILES: CC(=O)N1CCc2c(c(N3CCCc4cc(-c5cnn(C)c5)c(C(F)F)cc43)nn2CCOC2CCN(c3ncc(C(=O)N[C@H]4C(C)(C)[C@H](Oc5ccc(C#N)c(Cl)c5)C4(C)C)cn3)CC2)C1] |
| 52 | 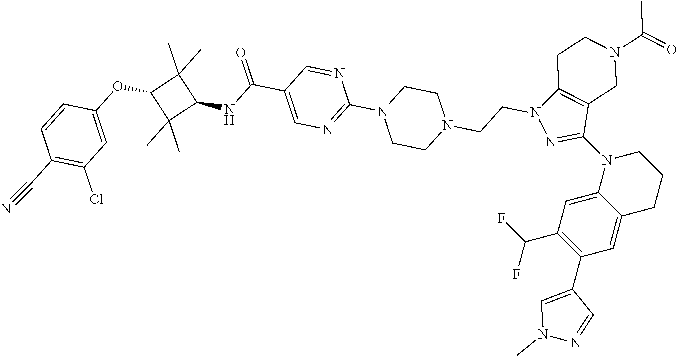 [SMILES: CC(=O)N1CCc2c(c(N3CCCc4cc(-c5cnn(C)c5)c(C(F)F)cc43)nn2CCN2CCN(c3ncc(C(=O)N[C@H]4C(C)(C)[C@H](Oc5ccc(C#N)c(Cl)c5)C4(C)C)cn3)CC2)C1] |
| 53 | 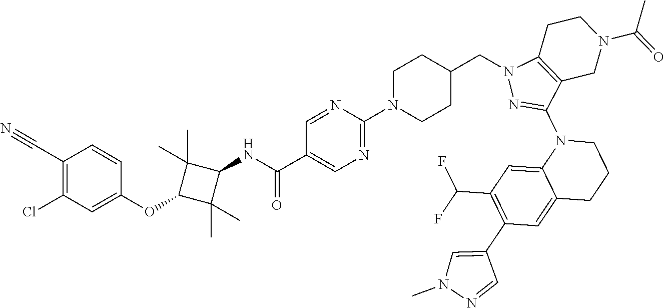 [SMILES: CC(=O)N1CCc2c(c(N3CCCc4cc(-c5cnn(C)c5)c(C(F)F)cc43)nn2CC2CCN(c3ncc(C(=O)N[C@H]4C(C)(C)[C@H](Oc5ccc(C#N)c(Cl)c5)C4(C)C)cn3)CC2)C1] |
| 54 | 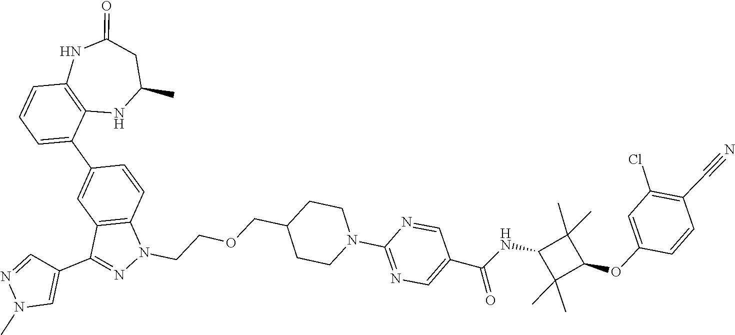 [SMILES: C[C@@H]1CC(=O)Nc2cccc(-c3ccc4c(c3)c(-c3cnn(C)c3)nn4CCOCC3CCN(c4ncc(C(=O)N[C@H]5C(C)(C)[C@H](Oc6ccc(C#N)c(Cl)c6)C5(C)C)cn4)CC3)c2N1] |
| 55 | 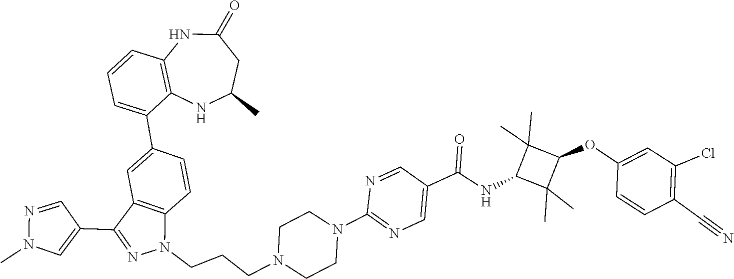 [SMILES: C[C@@H]1CC(=O)Nc2cccc(-c3ccc4c(c3)c(-c3cnn(C)c3)nn4CCCN3CCN(c4ncc(C(=O)N[C@H]5C(C)(C)[C@H](Oc6ccc(C#N)c(Cl)c6)C5(C)C)cn4)CC3)c2N1] |
| 56 | 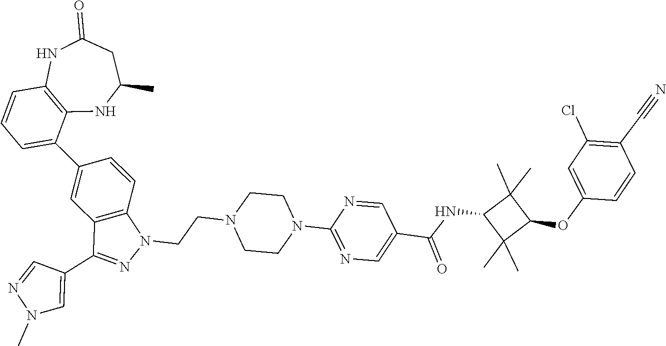 [SMILES: C[C@@H]1CC(=O)Nc2cccc(-c3ccc4c(c3)c(-c3cnn(C)c3)nn4CCN3CCN(c4ncc(C(=O)N[C@H]5C(C)(C)[C@H](Oc6ccc(C#N)c(Cl)c6)C5(C)C)cn4)CC3)c2N1] |
| 57 | 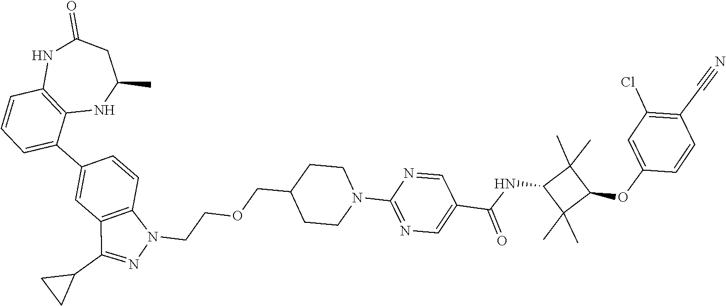 [SMILES: C[C@@H]1CC(=O)Nc2cccc(-c3ccc4c(c3)c(C3CC3)nn4CCOCC3CCN(c4ncc(C(=O)N[C@H]5C(C)(C)[C@H](Oc6ccc(C#N)c(Cl)c6)C5(C)C)cn4)CC3)c2N1] |
| 58 | 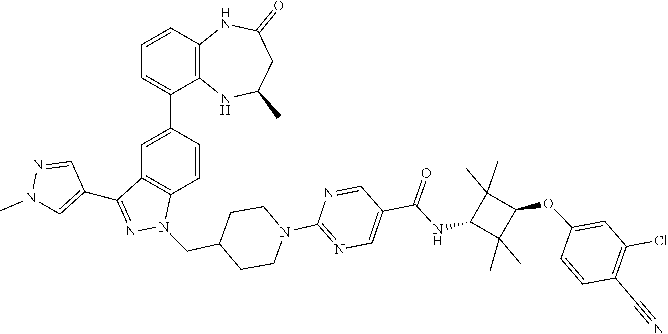 [SMILES: C[C@@H]1CC(=O)Nc2cccc(-c3ccc4c(c3)c(-c3cnn(C)c3)nn4CC3CCN(c4ncc(C(=O)N[C@H]5C(C)(C)[C@H](Oc6ccc(C#N)c(Cl)c6)C5(C)C)cn4)CC3)c2N1] |
| 59 | 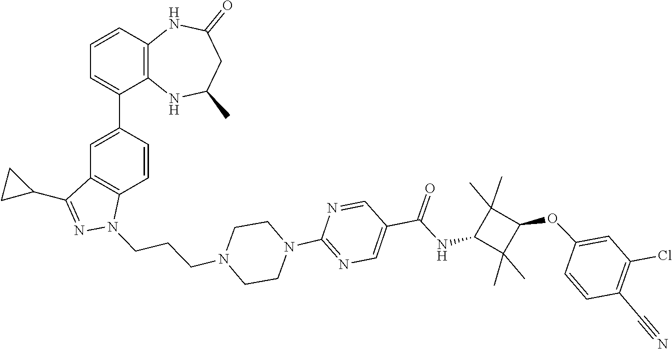 [SMILES: C[C@@H]1CC(=O)Nc2cccc(-c3ccc4c(c3)c(C3CC3)nn4CCCN3CCN(c4ncc(C(=O)N[C@H]5C(C)(C)[C@H](Oc6ccc(C#N)c(Cl)c6)C5(C)C)cn4)CC3)c2N1] |
| 60 | 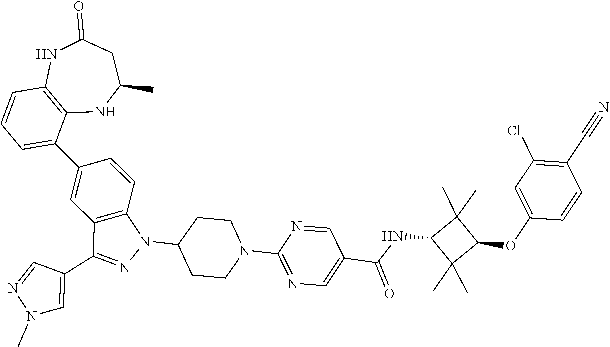 [SMILES: C[C@@H]1CC(=O)Nc2cccc(-c3ccc4c(c3)c(-c3cnn(C)c3)nn4C3CCN(c4ncc(C(=O)N[C@H]5C(C)(C)[C@H](Oc6ccc(C#N)c(Cl)c6)C5(C)C)cn4)CC3)c2N1] |
| 61 | 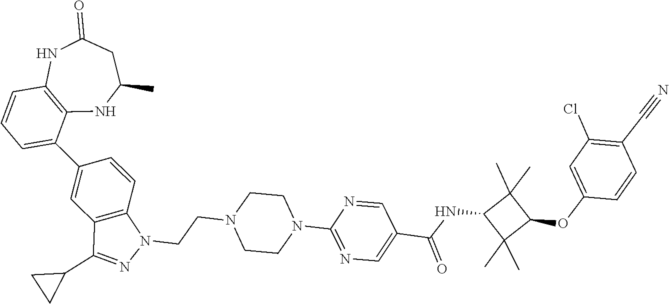 [SMILES: C[C@@H]1CC(=O)Nc2cccc(-c3ccc4c(c3)c(C3CC3)nn4CCN3CCN(c4ncc(C(=O)N[C@H]5C(C)(C)[C@H](Oc6ccc(C#N)c(Cl)c6)C5(C)C)cn4)CC3)c2N1] |
| 62 | 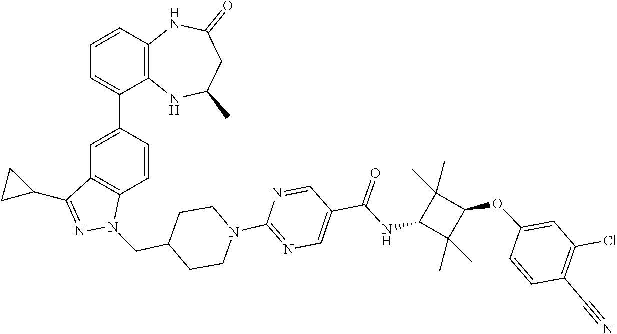 [SMILES: C[C@@H]1CC(=O)Nc2cccc(-c3ccc4c(c3)c(C3CC3)nn4CC3CCN(c4ncc(C(=O)N[C@H]5C(C)(C)[C@H](Oc6ccc(C#N)c(Cl)c6)C5(C)C)cn4)CC3)c2N1] |
| 63 | 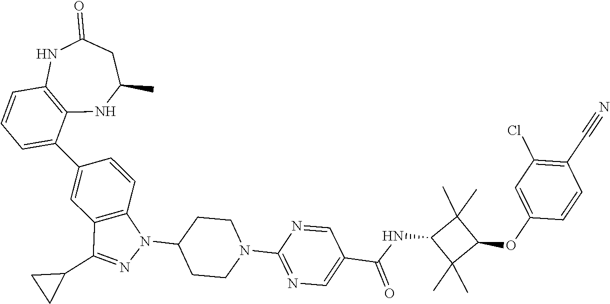 [SMILES: C[C@@H]1CC(=O)Nc2cccc(-c3ccc4c(c3)c(C3CC3)nn4C3CCN(c4ncc(C(=O)N[C@H]5C(C)(C)[C@H](Oc6ccc(C#N)c(Cl)c6)C5(C)C)cn4)CC3)c2N1] |
| 64 | 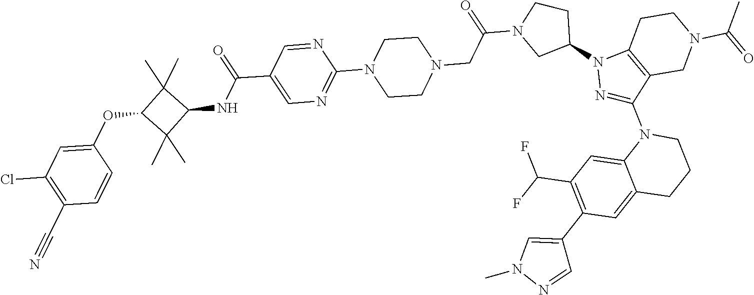 [SMILES: CC(=O)N1CCc2c(c(N3CCCc4cc(-c5cnn(C)c5)c(C(F)F)cc43)nn2[C@@H]2CCN(C(=O)CN3CCN(c4ncc(C(=O)N[C@H]5C(C)(C)[C@H](Oc6ccc(C#N)c(Cl)c6)C5(C)C)cn4)CC3)C2)C1] |
| 65 | 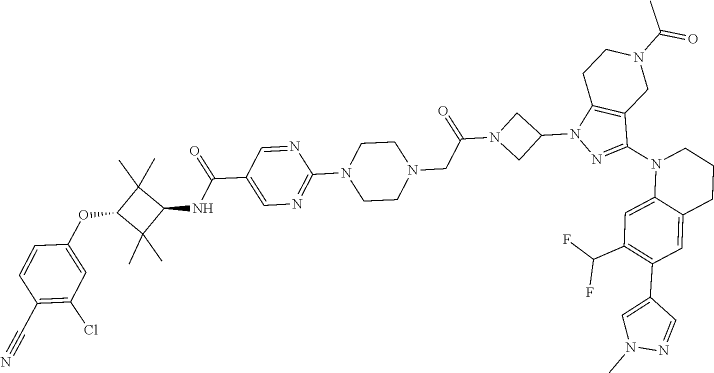 [SMILES: CC(=O)N1CCc2c(c(N3CCCc4cc(-c5cnn(C)c5)c(C(F)F)cc43)nn2C2CN(C(=O)CN3CCN(c4ncc(C(=O)N[C@H]5C(C)(C)[C@H](Oc6ccc(C#N)c(Cl)c6)C5(C)C)cn4)CC3)C2)C1] |
| 66 | 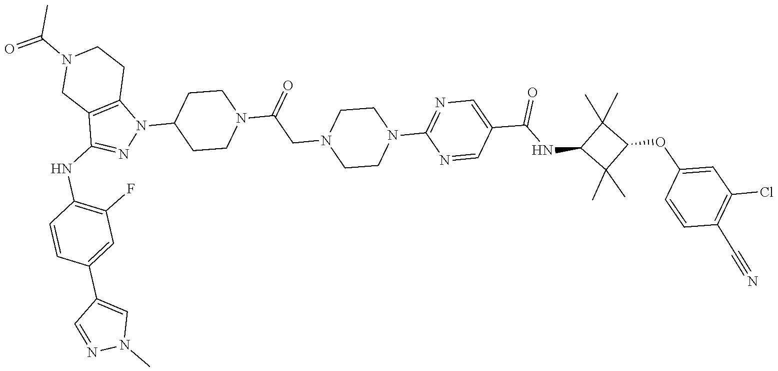 [SMILES: CC(=O)N1CCc2c(c(Nc3ccc(-c4cnn(C)c4)cc3F)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1] |
| 67 | 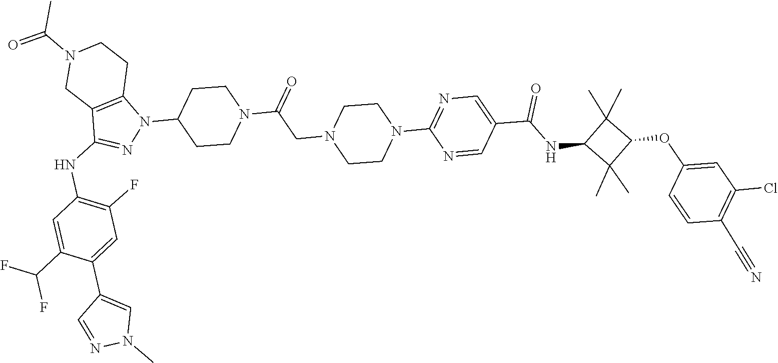 [SMILES: CC(=O)N1CCc2c(c(Nc3cc(C(F)F)c(-c4cnn(C)c4)cc3F)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1] |
| 68 | 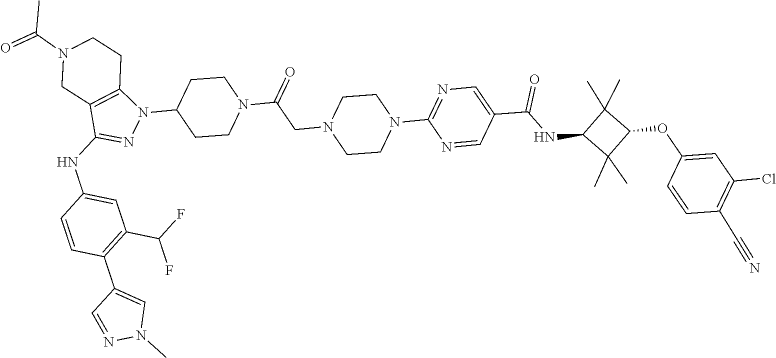 [SMILES: CC(=O)N1CCc2c(c(Nc3ccc(-c4cnn(C)c4)c(C(F)F)c3)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1] |
| 69 | 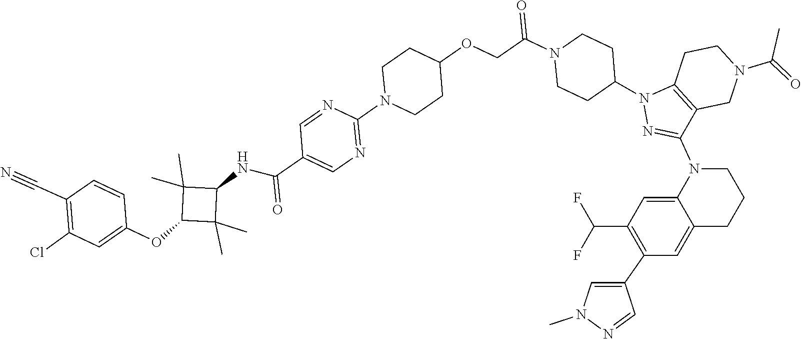 [SMILES: CC(=O)N1CCc2c(c(N3CCCc4cc(-c5cnn(C)c5)c(C(F)F)cc43)nn2C2CCN(C(=O)COC3CCN(c4ncc(C(=O)N[C@H]5C(C)(C)[C@H](Oc6ccc(C#N)c(Cl)c6)C5(C)C)cn4)CC3)CC2)C1] |
| 70 | 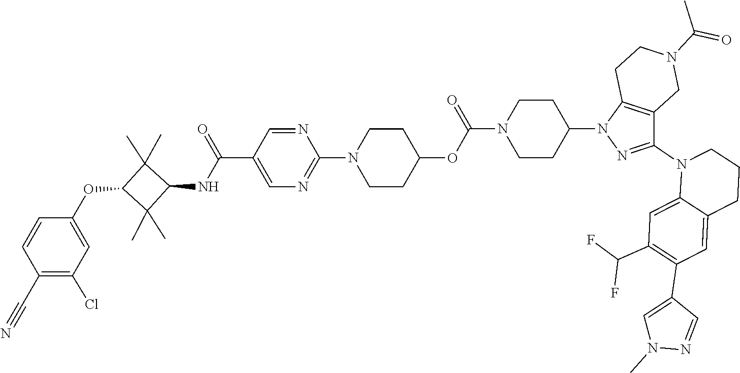 [SMILES: CC(=O)N1CCc2c(c(N3CCCc4cc(-c5cnn(C)c5)c(C(F)F)cc43)nn2C2CCN(C(=O)OC3CCN(c4ncc(C(=O)N[C@H]5C(C)(C)[C@H](Oc6ccc(C#N)c(Cl)c6)C5(C)C)cn4)CC3)CC2)C1] |
| 71 | 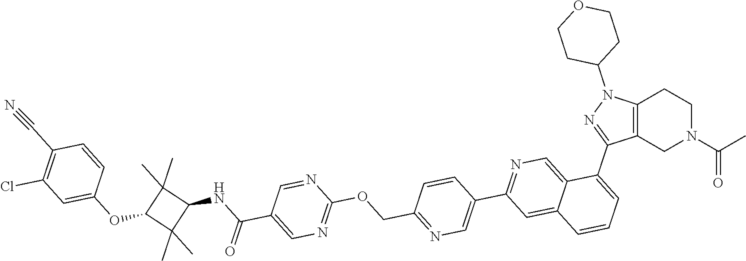 [SMILES: CC(=O)N1CCc2c(c(-c3cccc4cc(-c5ccc(COc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 72 | 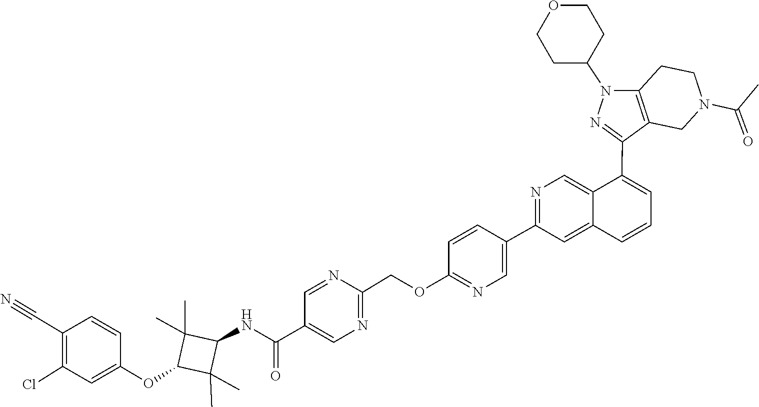 [SMILES: CC(=O)N1CCc2c(c(-c3cccc4cc(-c5ccc(OCc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 73 | 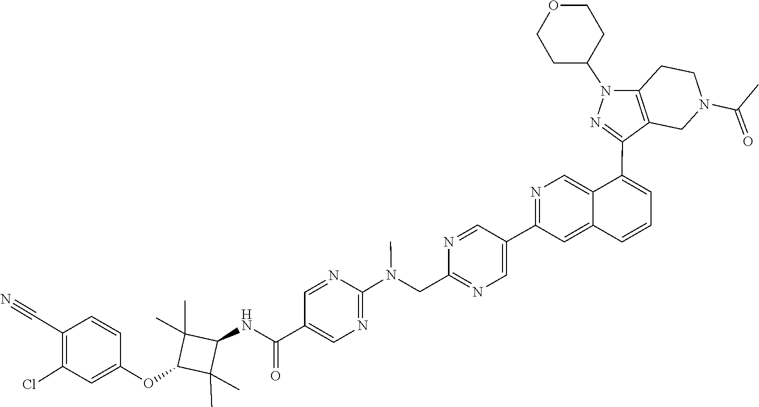 [SMILES: CC(=O)N1CCc2c(c(-c3cccc4cc(-c5cnc(CN(C)c6ncc(C(=O)N[C@H]7C(C)(C)[C@H](Oc8ccc(C#N)c(Cl)c8)C7(C)C)cn6)nc5)ncc34)nn2C2CCOCC2)C1] |
| 74 | 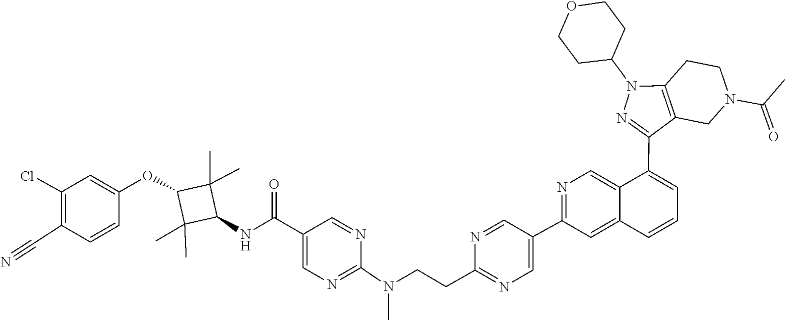 [SMILES: CC(=O)N1CCc2c(c(-c3cccc4cc(-c5cnc(CCN(C)c6ncc(C(=O)N[C@H]7C(C)(C)[C@H](Oc8ccc(C#N)c(Cl)c8)C7(C)C)cn6)nc5)ncc34)nn2C2CCOCC2)C1] |
| 75 | 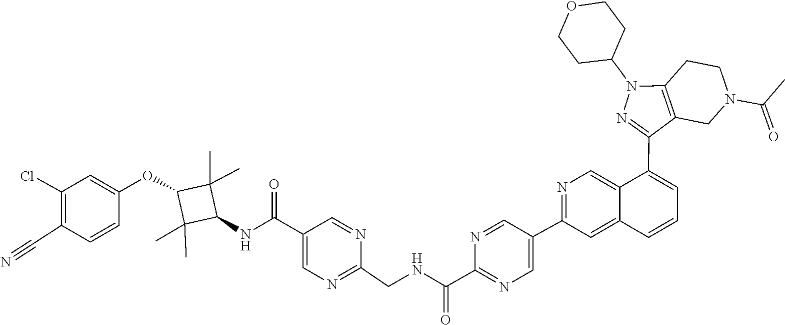 [SMILES: CC(=O)N1CCc2c(c(-c3cccc4cc(-c5cnc(C(=O)NCc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 76 | 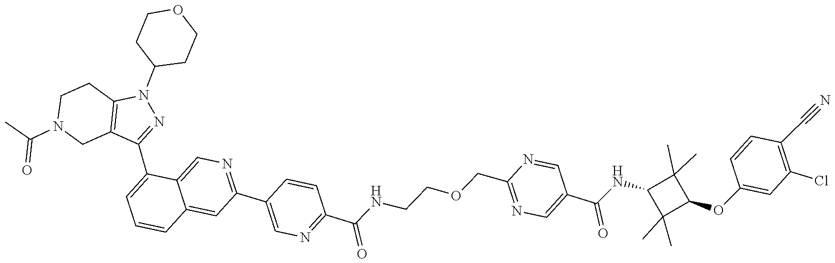 [SMILES: CC(=O)N1CCc2c(c(-c3cccc4cc(-c5ccc(C(=O)NCCOCc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 77 | 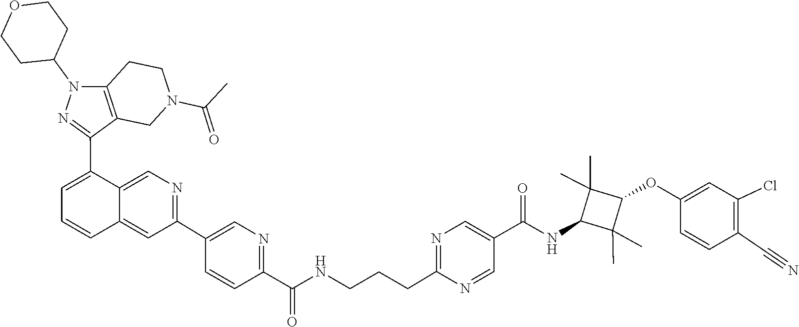 [SMILES: CC(=O)N1CCc2c(c(-c3cccc4cc(-c5ccc(C(=O)NCCCc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 78 | 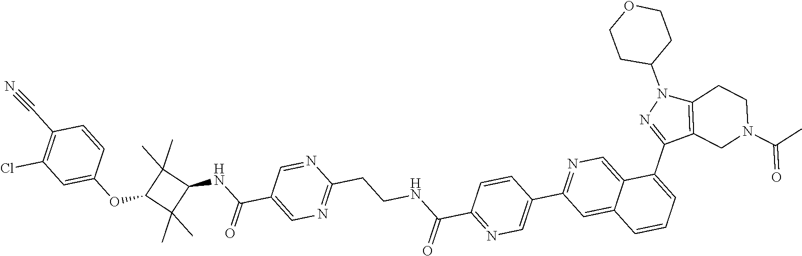 [SMILES: CC(=O)N1CCc2c(c(-c3cccc4cc(-c5ccc(C(=O)NCCc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 79 | 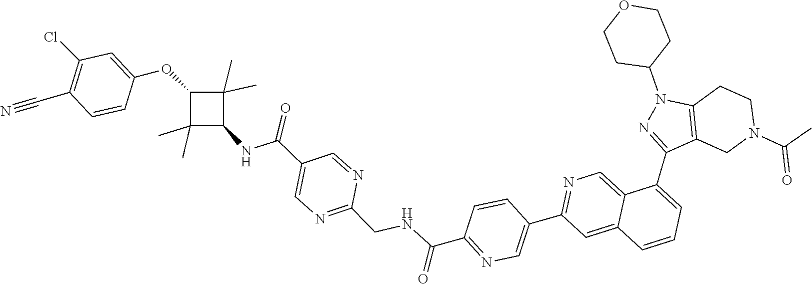 [SMILES: CC(=O)N1CCc2c(c(-c3cccc4cc(-c5ccc(C(=O)NCc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 80 | 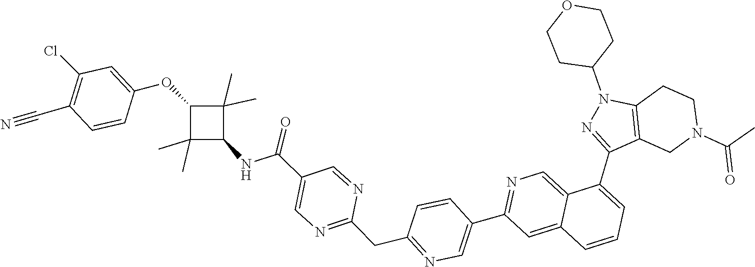 [SMILES: CC(=O)N1CCc2c(c(-c3cccc4cc(-c5ccc(Cc6ncc(C(=O)N[C@H]7C(C)(C)[C@H](Oc8ccc(C#N)c(Cl)c8)C7(C)C)cn6)nc5)ncc34)nn2C2CCOCC2)C1] |
| 81 | 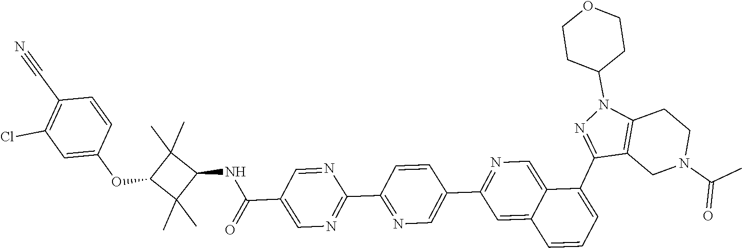 [SMILES: CC(=O)N1CCc2c(c(-c3cccc4cc(-c5ccc(-c6ncc(C(=O)N[C@H]7C(C)(C)[C@H](Oc8ccc(C#N)c(Cl)c8)C7(C)C)cn6)nc5)ncc34)nn2C2CCOCC2)C1] |
| 82 | 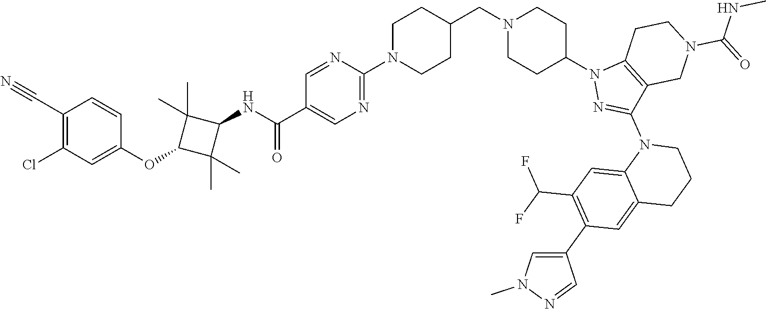 [SMILES: CNC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 83 | 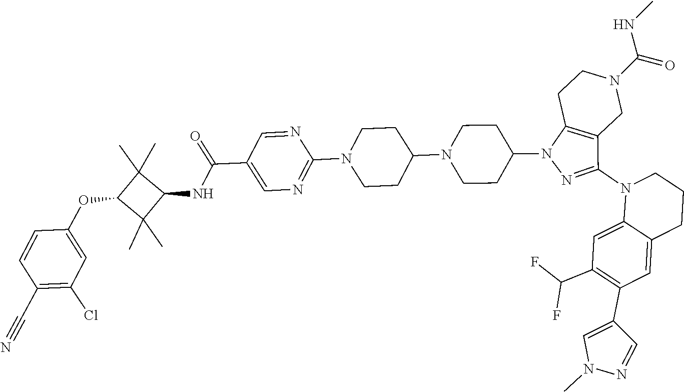 [SMILES: CNC(=O)N1CCc2c(c(N3CCCc4cc(-c5cnn(C)c5)c(C(F)F)cc43)nn2C2CCN(C3CCN(c4ncc(C(=O)N[C@H]5C(C)(C)[C@H](Oc6ccc(C#N)c(Cl)c6)C5(C)C)cn4)CC3)CC2)C1] |
| 84 | 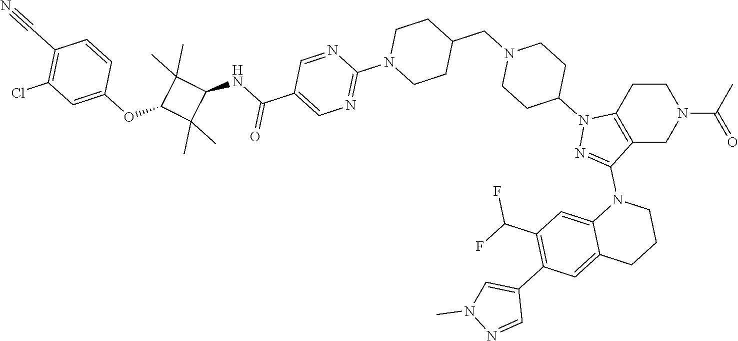 [SMILES: CC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 85 | 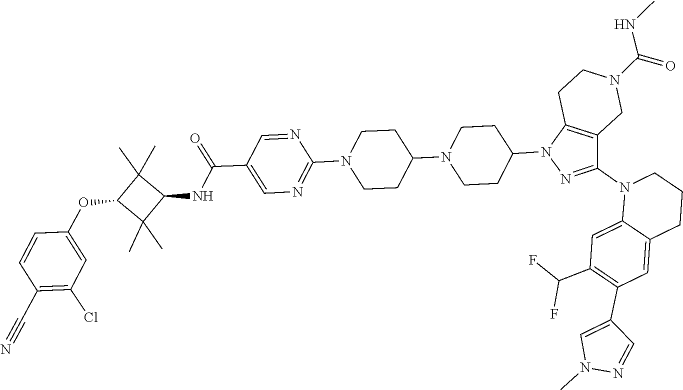 [SMILES: CNC(=O)N1CCc2c(c(N3CCCc4cc(-c5cnn(C)c5)c(C(F)F)cc43)nn2C2CCN(C3CCN(c4ncc(C(=O)N[C@H]5C(C)(C)[C@H](Oc6ccc(C#N)c(Cl)c6)C5(C)C)cn4)CC3)CC2)C1] |
| 86 | 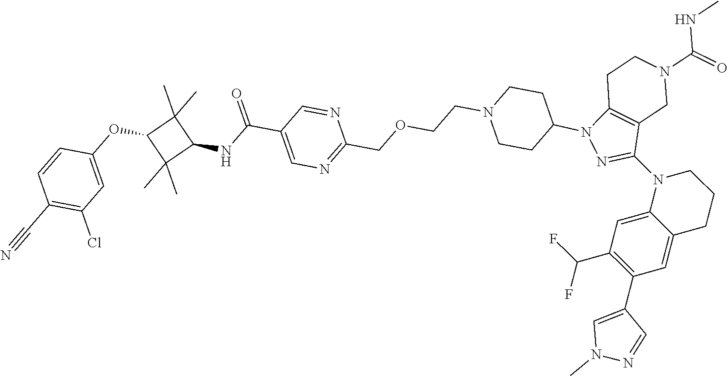 [SMILES: CNC(=O)N1CCc2c(c(N3CCCc4cc(-c5cnn(C)c5)c(C(F)F)cc43)nn2C2CCN(CCOCc3ncc(C(=O)N[C@H]4C(C)(C)[C@H](Oc5ccc(C#N)c(Cl)c5)C4(C)C)cn3)CC2)C1] |
| 87 | 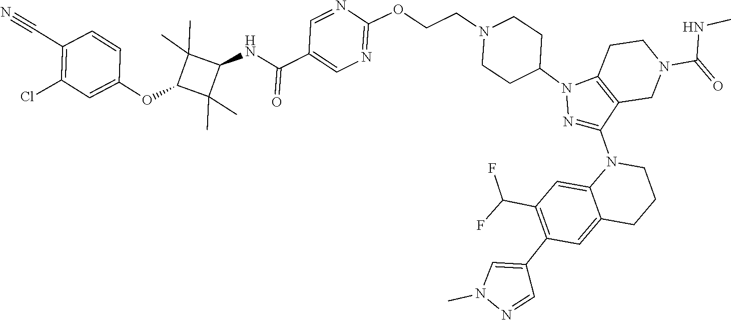 [SMILES: CNC(=O)N1CCc2c(c(N3CCCc4cc(-c5cnn(C)c5)c(C(F)F)cc43)nn2C2CCN(CCOc3ncc(C(=O)N[C@H]4C(C)(C)[C@H](Oc5ccc(C#N)c(Cl)c5)C4(C)C)cn3)CC2)C1] |
| 88 | 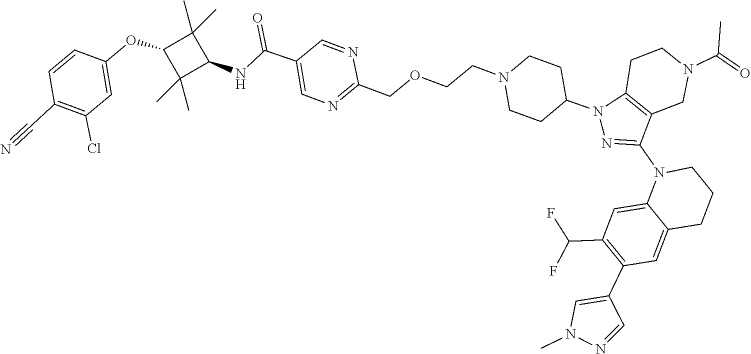 [SMILES: CC(=O)N1CCc2c(c(N3CCCc4cc(-c5cnn(C)c5)c(C(F)F)cc43)nn2C2CCN(CCOCc3ncc(C(=O)N[C@H]4C(C)(C)[C@H](Oc5ccc(C#N)c(Cl)c5)C4(C)C)cn3)CC2)C1] |
| 89 | 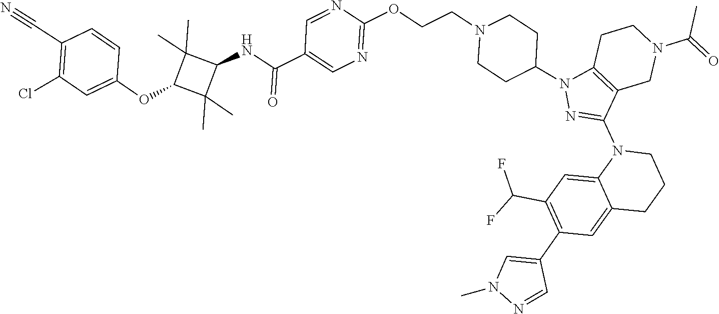 [SMILES: CC(=O)N1CCc2c(c(N3CCCc4cc(-c5cnn(C)c5)c(C(F)F)cc43)nn2C2CCN(CCOc3ncc(C(=O)N[C@H]4C(C)(C)[C@H](Oc5ccc(C#N)c(Cl)c5)C4(C)C)cn3)CC2)C1] |
| 90 | 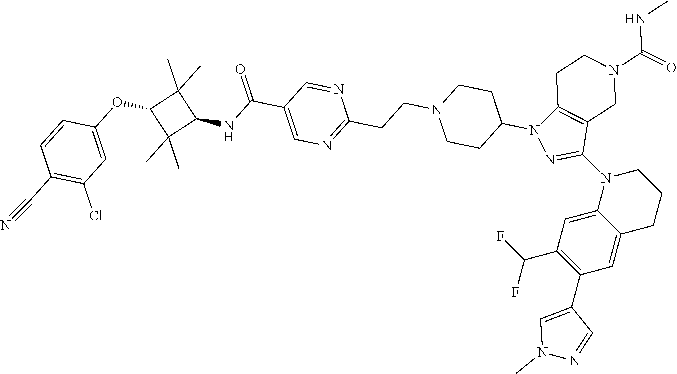 [SMILES: CNC(=O)N1CCc2c(c(N3CCCc4cc(-c5cnn(C)c5)c(C(F)F)cc43)nn2C2CCN(CCc3ncc(C(=O)N[C@H]4C(C)(C)[C@H](Oc5ccc(C#N)c(Cl)c5)C4(C)C)cn3)CC2)C1] |
| 91 | 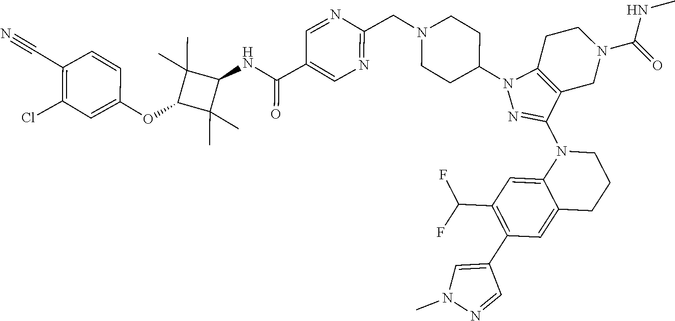 [SMILES: CNC(=O)N1CCc2c(c(N3CCCc4cc(-c5cnn(C)c5)c(C(F)F)cc43)nn2C2CCN(Cc3ncc(C(=O)N[C@H]4C(C)(C)[C@H](Oc5ccc(C#N)c(Cl)c5)C4(C)C)cn3)CC2)C1] |
| 92 | 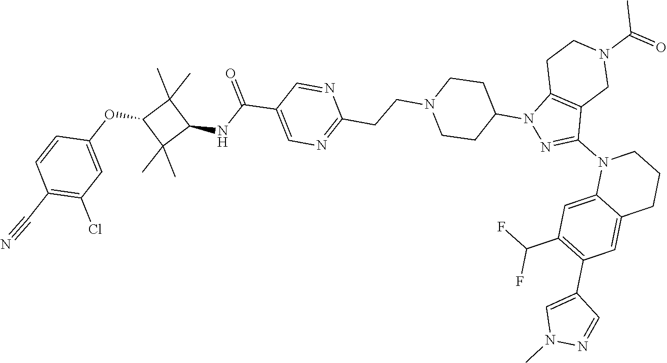 [SMILES: CC(=O)N1CCc2c(c(N3CCCc4cc(-c5cnn(C)c5)c(C(F)F)cc43)nn2C2CCN(CCc3ncc(C(=O)N[C@H]4C(C)(C)[C@H](Oc5ccc(C#N)c(Cl)c5)C4(C)C)cn3)CC2)C1] |
| 93 | 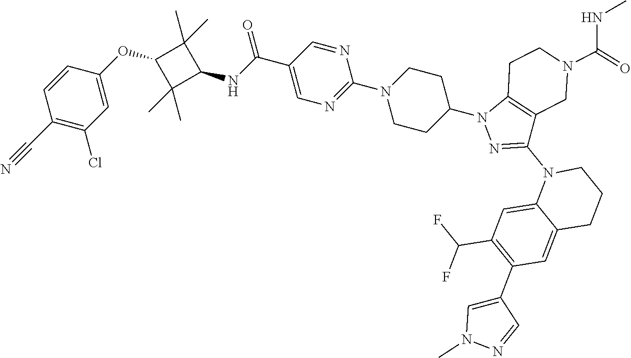 [SMILES: CNC(=O)N1CCc2c(c(N3CCCc4cc(-c5cnn(C)c5)c(C(F)F)cc43)nn2C2CCN(c3ncc(C(=O)N[C@H]4C(C)(C)[C@H](Oc5ccc(C#N)c(Cl)c5)C4(C)C)cn3)CC2)C1] |
| 94 | 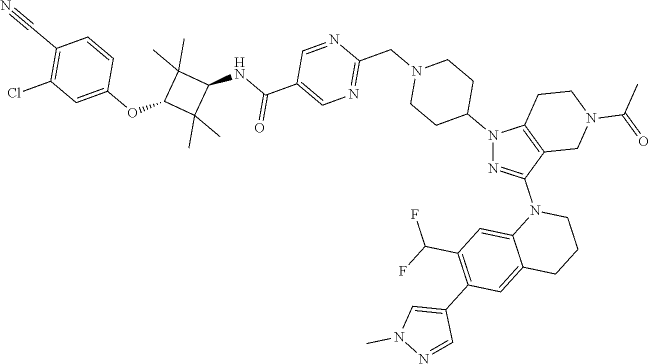 [SMILES: CC(=O)N1CCc2c(c(N3CCCc4cc(-c5cnn(C)c5)c(C(F)F)cc43)nn2C2CCN(Cc3ncc(C(=O)N[C@H]4C(C)(C)[C@H](Oc5ccc(C#N)c(Cl)c5)C4(C)C)cn3)CC2)C1] |
| 95 | 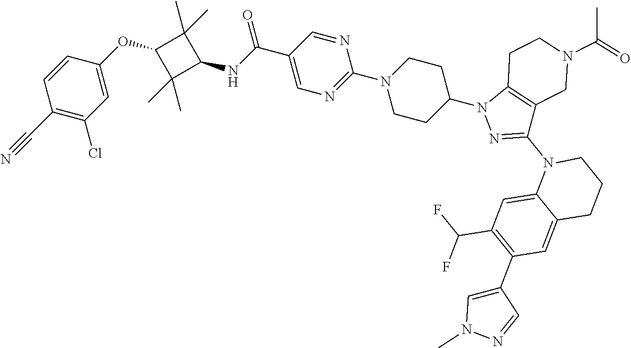 [SMILES: CC(=O)N1CCc2c(c(N3CCCc4cc(-c5cnn(C)c5)c(C(F)F)cc43)nn2C2CCN(c3ncc(C(=O)N[C@H]4C(C)(C)[C@H](Oc5ccc(C#N)c(Cl)c5)C4(C)C)cn3)CC2)C1] |
| 96 | 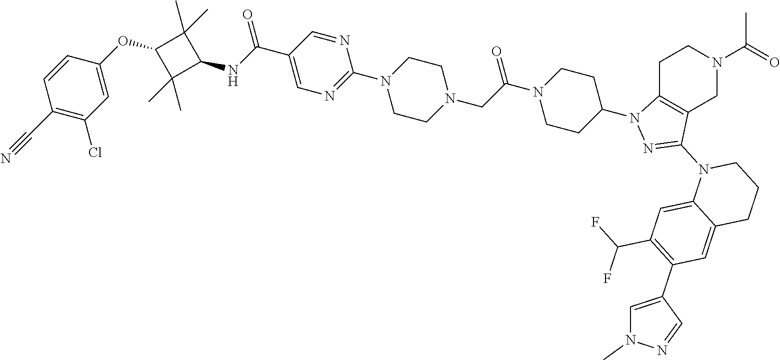 [SMILES: CC(=O)N1CCc2c(c(N3CCCc4cc(-c5cnn(C)c5)c(C(F)F)cc43)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1] |
| 97 | 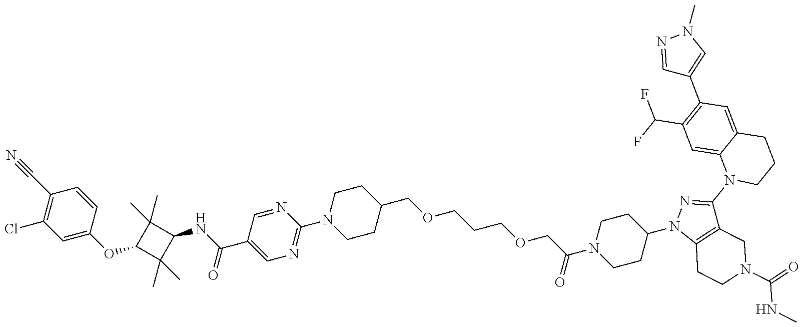 [SMILES: CNC(=O)N1CCc2c(c(N3CCCc4cc(-c5cnn(C)c5)c(C(F)F)cc43)nn2C2CCN(C(=O)COCCCOCC3CCN(c4ncc(C(=O)N[C@H]5C(C)(C)[C@H](Oc6ccc(C#N)c(Cl)c6)C5(C)C)cn4)CC3)CC2)C1] |
| 98 | 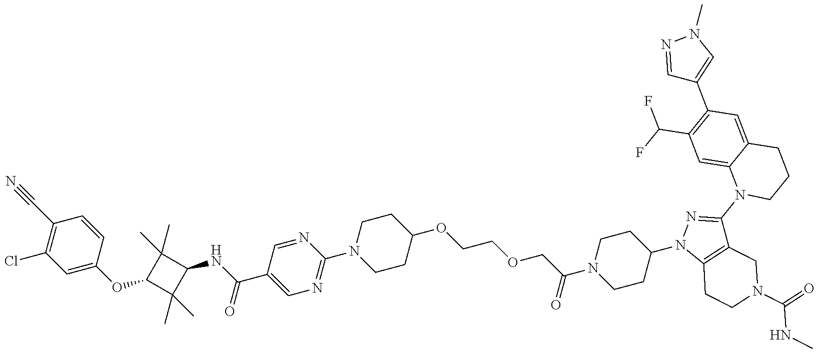 [SMILES: CNC(=O)N1CCc2c(c(N3CCCc4cc(-c5cnn(C)c5)c(C(F)F)cc43)nn2C2CCN(C(=O)COCCOC3CCN(c4ncc(C(=O)N[C@H]5C(C)(C)[C@H](Oc6ccc(C#N)c(Cl)c6)C5(C)C)cn4)CC3)CC2)C1] |
| 99 | 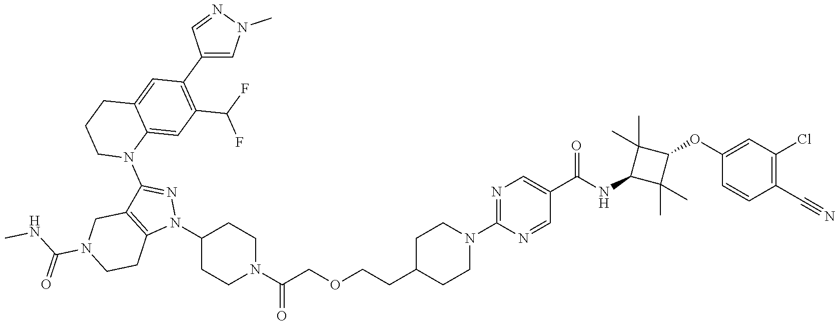 [SMILES: CNC(=O)N1CCc2c(c(N3CCCc4cc(-c5cnn(C)c5)c(C(F)F)cc43)nn2C2CCN(C(=O)COCCC3CCN(c4ncc(C(=O)N[C@H]5C(C)(C)[C@H](Oc6ccc(C#N)c(Cl)c6)C5(C)C)cn4)CC3)CC2)C1] |
| 100 | 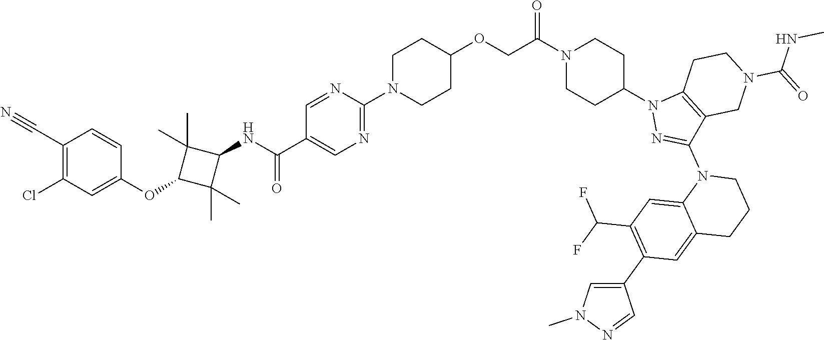 [SMILES: CNC(=O)N1CCc2c(c(N3CCCc4cc(-c5cnn(C)c5)c(C(F)F)cc43)nn2C2CCN(C(=O)COC3CCN(c4ncc(C(=O)N[C@H]5C(C)(C)[C@H](Oc6ccc(C#N)c(Cl)c6)C5(C)C)cn4)CC3)CC2)C1] |
| 101 | 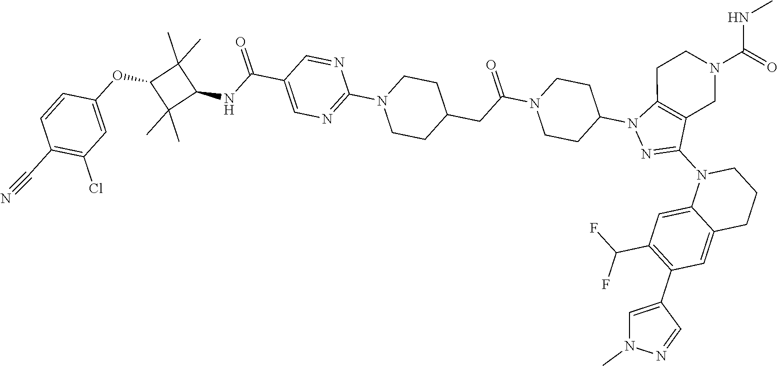 [SMILES: CNC(=O)N1CCc2c(c(N3CCCc4cc(-c5cnn(C)c5)c(C(F)F)cc43)nn2C2CCN(C(=O)CC3CCN(c4ncc(C(=O)N[C@H]5C(C)(C)[C@H](Oc6ccc(C#N)c(Cl)c6)C5(C)C)cn4)CC3)CC2)C1] |
| 102 | 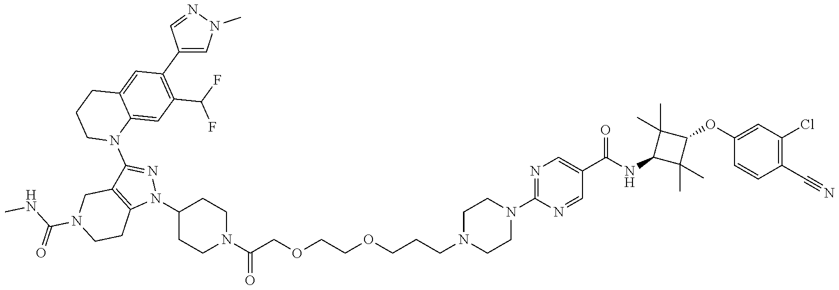 [SMILES: CNC(=O)N1CCc2c(c(N3CCCc4cc(-c5cnn(C)c5)c(C(F)F)cc43)nn2C2CCN(C(=O)COCCOCCCN3CCN(c4ncc(C(=O)N[C@H]5C(C)(C)[C@H](Oc6ccc(C#N)c(Cl)c6)C5(C)C)cn4)CC3)CC2)C1] |
| 103 | 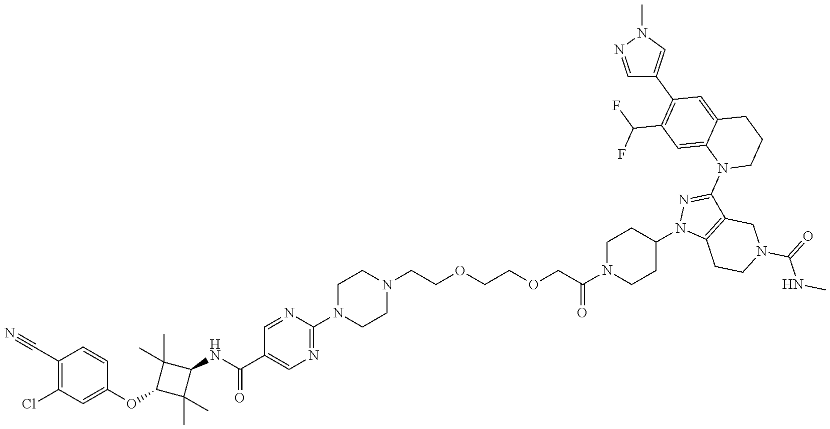 [SMILES: CNC(=O)N1CCc2c(c(N3CCCc4cc(-c5cnn(C)c5)c(C(F)F)cc43)nn2C2CCN(C(=O)COCCOCCN3CCN(c4ncc(C(=O)N[C@H]5C(C)(C)[C@H](Oc6ccc(C#N)c(Cl)c6)C5(C)C)cn4)CC3)CC2)C1] |
| 104 | 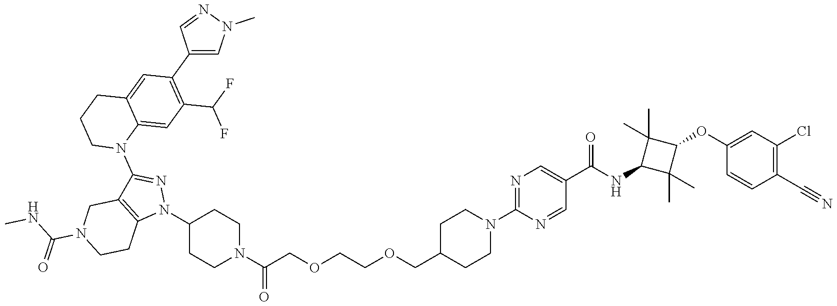 [SMILES: CNC(=O)N1CCc2c(c(N3CCCc4cc(-c5cnn(C)c5)c(C(F)F)cc43)nn2C2CCN(C(=O)COCCOCC3CCN(c4ncc(C(=O)N[C@H]5C(C)(C)[C@H](Oc6ccc(C#N)c(Cl)c6)C5(C)C)cn4)CC3)CC2)C1] |
| 105 | 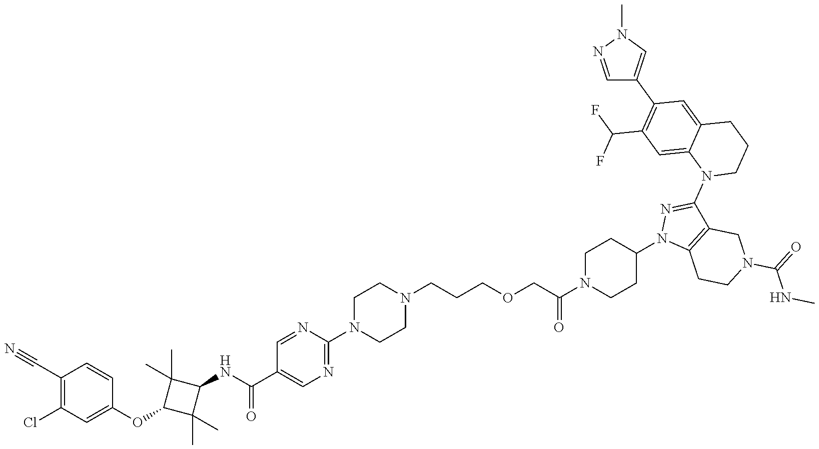 [SMILES: CNC(=O)N1CCc2c(c(N3CCCc4cc(-c5cnn(C)c5)c(C(F)F)cc43)nn2C2CCN(C(=O)COCCCN3CCN(c4ncc(C(=O)N[C@H]5C(C)(C)[C@H](Oc6ccc(C#N)c(Cl)c6)C5(C)C)cn4)CC3)CC2)C1] |
| 106 | 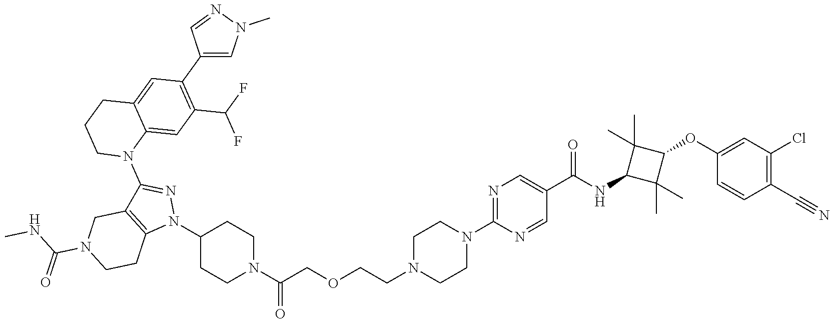 [SMILES: CNC(=O)N1CCc2c(c(N3CCCc4cc(-c5cnn(C)c5)c(C(F)F)cc43)nn2C2CCN(C(=O)COCCN3CCN(c4ncc(C(=O)N[C@H]5C(C)(C)[C@H](Oc6ccc(C#N)c(Cl)c6)C5(C)C)cn4)CC3)CC2)C1] |
| 107 | 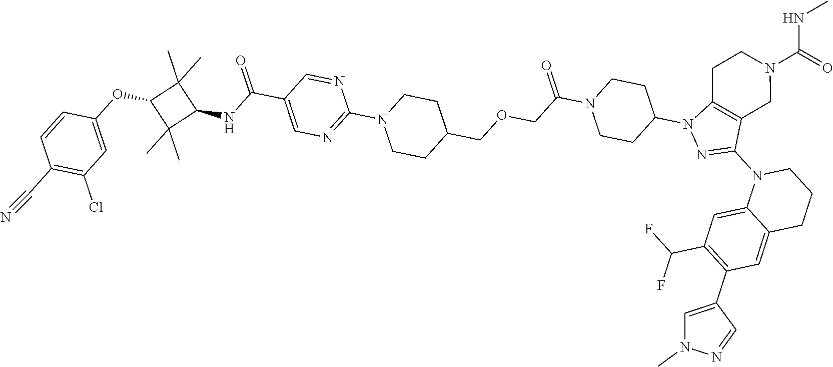 [SMILES: CNC(=O)N1CCc2c(c(N3CCCc4cc(-c5cnn(C)c5)c(C(F)F)cc43)nn2C2CCN(C(=O)COCC3CCN(c4ncc(C(=O)N[C@H]5C(C)(C)[C@H](Oc6ccc(C#N)c(Cl)c6)C5(C)C)cn4)CC3)CC2)C1] |
| 108 | 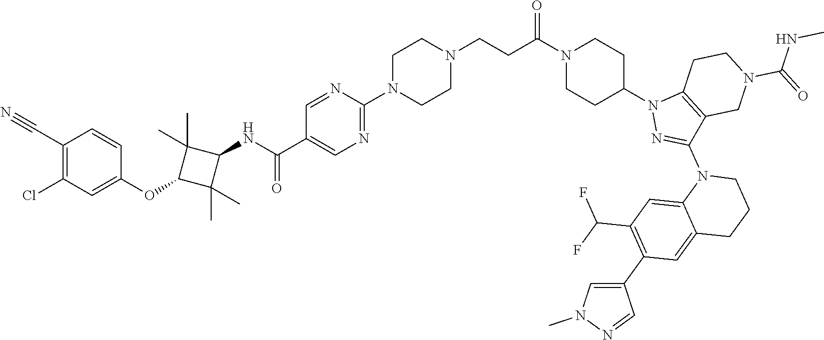 [SMILES: CNC(=O)N1CCc2c(c(N3CCCc4cc(-c5cnn(C)c5)c(C(F)F)cc43)nn2C2CCN(C(=O)CCN3CCN(c4ncc(C(=O)N[C@H]5C(C)(C)[C@H](Oc6ccc(C#N)c(Cl)c6)C5(C)C)cn4)CC3)CC2)C1] |
| 109 | 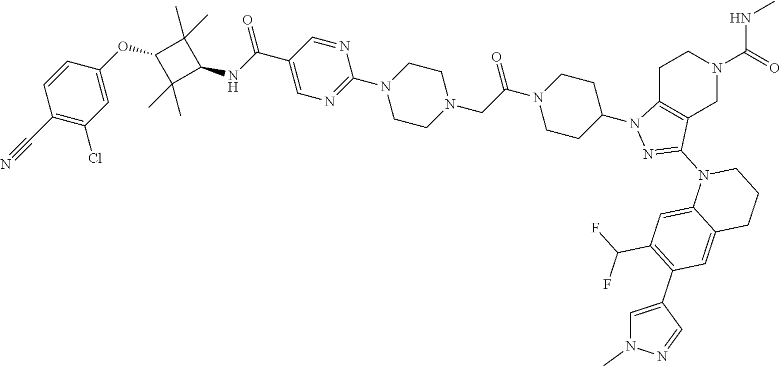 [SMILES: CNC(=O)N1CCc2c(c(N3CCCc4cc(-c5cnn(C)c5)c(C(F)F)cc43)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1] |
| 110 | 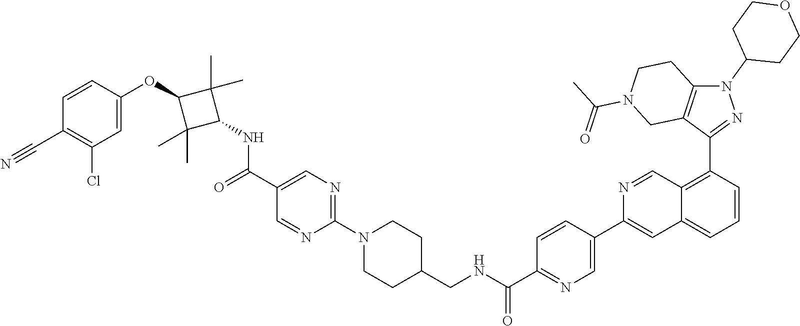 [SMILES: CC(=O)N1CCc2c(c(-c3cccc4cc(-c5ccc(C(=O)NCC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 111 | 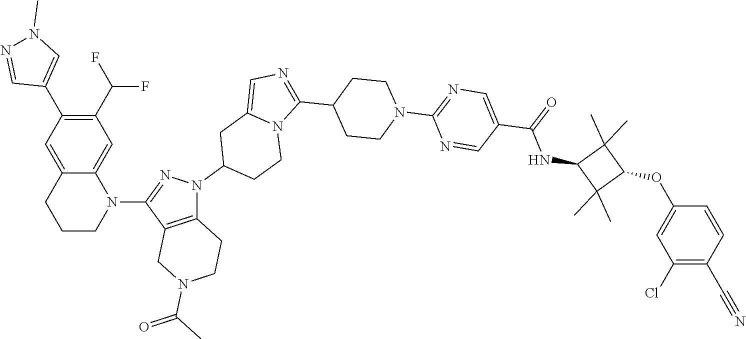 [SMILES: CC(=O)N1CCc2c(c(N3CCCc4cc(-c5cnn(C)c5)c(C(F)F)cc43)nn2C2CCn3c(cnc3C3CCN(c4ncc(C(=O)N[C@H]5C(C)(C)[C@H](Oc6ccc(C#N)c(Cl)c6)C5(C)C)cn4)CC3)C2)C1] |
| 112 | 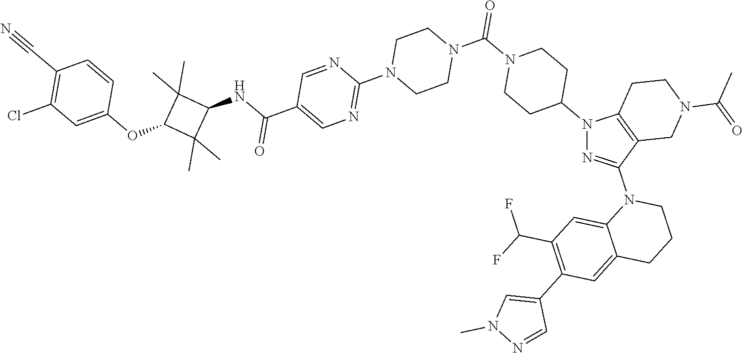 [SMILES: CC(=O)N1CCc2c(c(N3CCCc4cc(-c5cnn(C)c5)c(C(F)F)cc43)nn2C2CCN(C(=O)N3CCN(c4ncc(C(=O)N[C@H]5C(C)(C)[C@H](Oc6ccc(C#N)c(Cl)c6)C5(C)C)cn4)CC3)CC2)C1] |
| 113 | 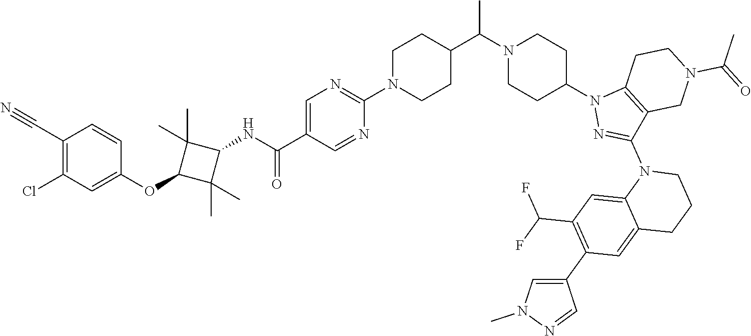 [SMILES: CC(=O)N1CCc2c(c(N3CCCc4cc(-c5cnn(C)c5)c(C(F)F)cc43)nn2C2CCN(C(C)C3CCN(c4ncc(C(=O)N[C@H]5C(C)(C)[C@H](Oc6ccc(C#N)c(Cl)c6)C5(C)C)cn4)CC3)CC2)C1] |
| 114 | 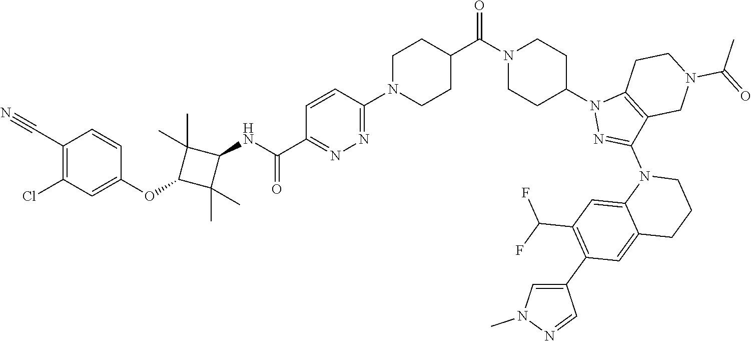 [SMILES: CC(=O)N1CCc2c(c(N3CCCc4cc(-c5cnn(C)c5)c(C(F)F)cc43)nn2C2CCN(C(=O)C3CCN(c4ccc(C(=O)N[C@H]5C(C)(C)[C@H](Oc6ccc(C#N)c(Cl)c6)C5(C)C)nn4)CC3)CC2)C1] |
| 115 | 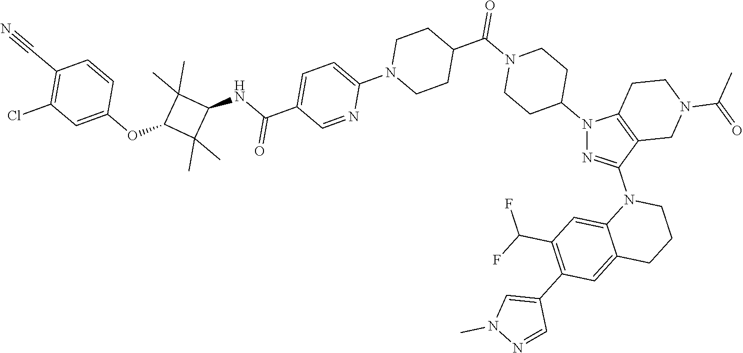 [SMILES: CC(=O)N1CCc2c(c(N3CCCc4cc(-c5cnn(C)c5)c(C(F)F)cc43)nn2C2CCN(C(=O)C3CCN(c4ccc(C(=O)N[C@H]5C(C)(C)[C@H](Oc6ccc(C#N)c(Cl)c6)C5(C)C)cn4)CC3)CC2)C1] |
| 116 | 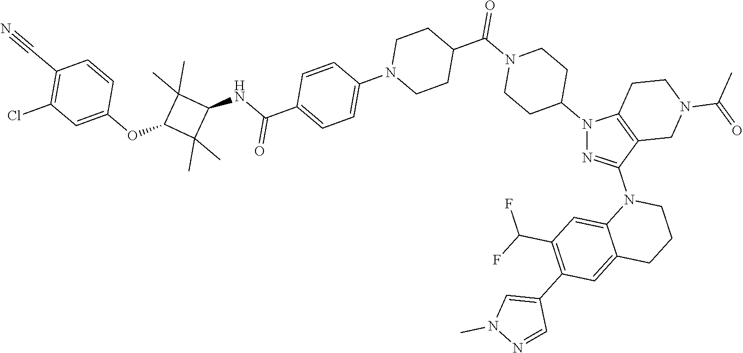 [SMILES: CC(=O)N1CCc2c(c(N3CCCc4cc(-c5cnn(C)c5)c(C(F)F)cc43)nn2C2CCN(C(=O)C3CCN(c4ccc(C(=O)N[C@H]5C(C)(C)[C@H](Oc6ccc(C#N)c(Cl)c6)C5(C)C)cc4)CC3)CC2)C1] |
| 117 | 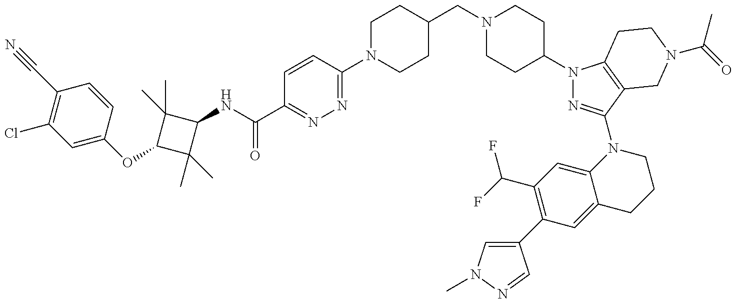 [SMILES: CC(=O)N1CCc2c(c(N3CCCc4cc(-c5cnn(C)c5)c(C(F)F)cc43)nn2C2CCN(CC3CCN(c4ccc(C(=O)N[C@H]5C(C)(C)[C@H](Oc6ccc(C#N)c(Cl)c6)C5(C)C)nn4)CC3)CC2)C1] |
| 118 | 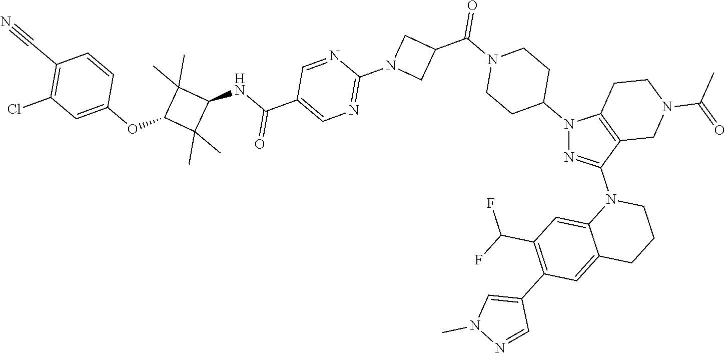 [SMILES: CC(=O)N1CCc2c(c(N3CCCc4cc(-c5cnn(C)c5)c(C(F)F)cc43)nn2C2CCN(C(=O)C3CN(c4ncc(C(=O)N[C@H]5C(C)(C)[C@H](Oc6ccc(C#N)c(Cl)c6)C5(C)C)cn4)C3)CC2)C1] |
| 119 | 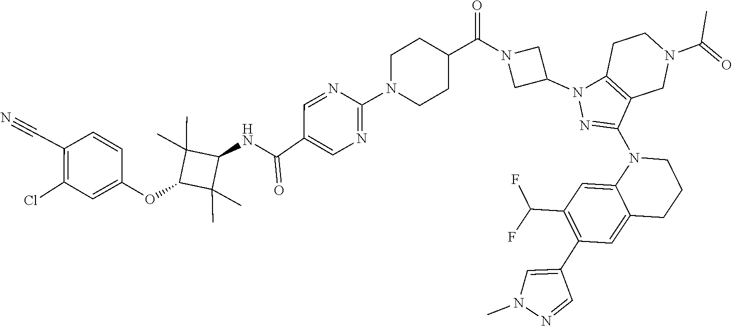 [SMILES: CC(=O)N1CCc2c(c(N3CCCc4cc(-c5cnn(C)c5)c(C(F)F)cc43)nn2C2CN(C(=O)C3CCN(c4ncc(C(=O)N[C@H]5C(C)(C)[C@H](Oc6ccc(C#N)c(Cl)c6)C5(C)C)cn4)CC3)C2)C1] |
| 120 | 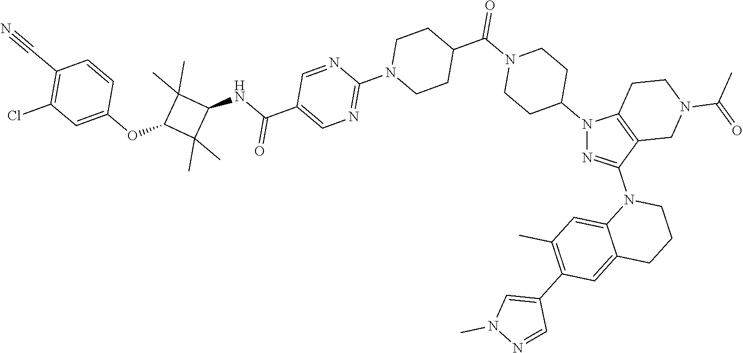 [SMILES: CC(=O)N1CCc2c(c(N3CCCc4cc(-c5cnn(C)c5)c(C)cc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1] |
| 121 | 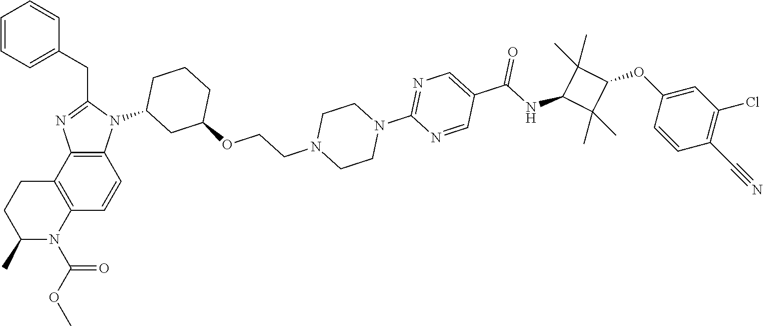 [SMILES: COC(=O)N1c2ccc3c(nc(Cc4ccccc4)n3[C@@H]3CCC[C@@H](OCCN4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 122 | 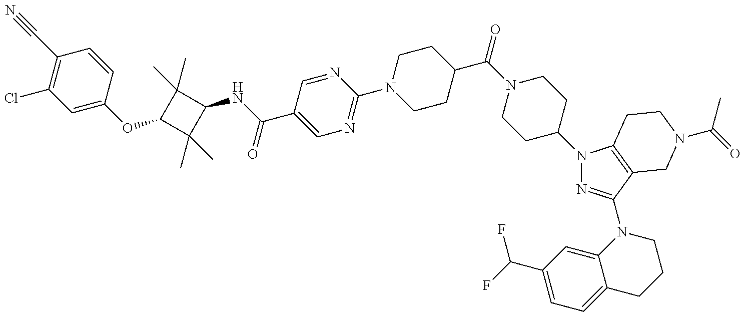 [SMILES: CC(=O)N1CCc2c(c(N3CCCc4ccc(C(F)F)cc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1] |
| 123 | 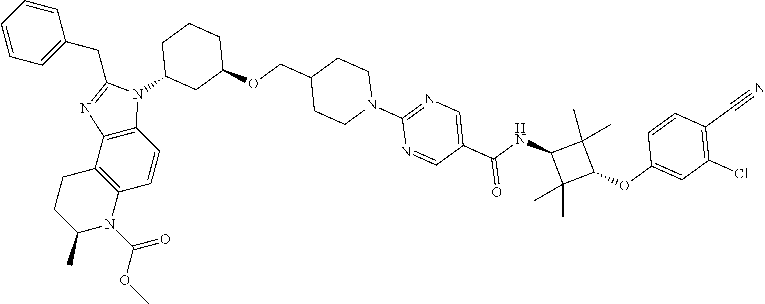 [SMILES: COC(=O)N1c2ccc3c(nc(Cc4ccccc4)n3[C@@H]3CCC[C@@H](OCC4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 124 | 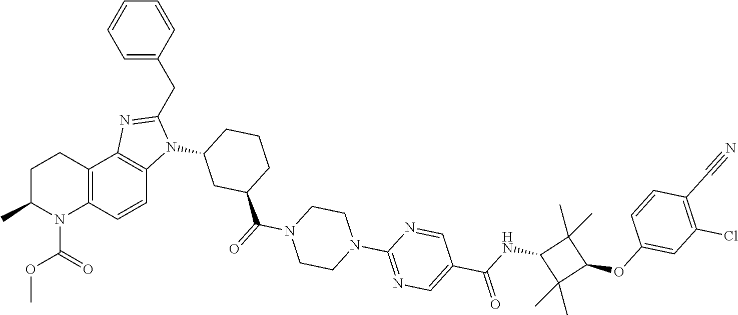 [SMILES: COC(=O)N1c2ccc3c(nc(Cc4ccccc4)n3[C@@H]3CCC[C@@H](C(=O)N4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 125 | 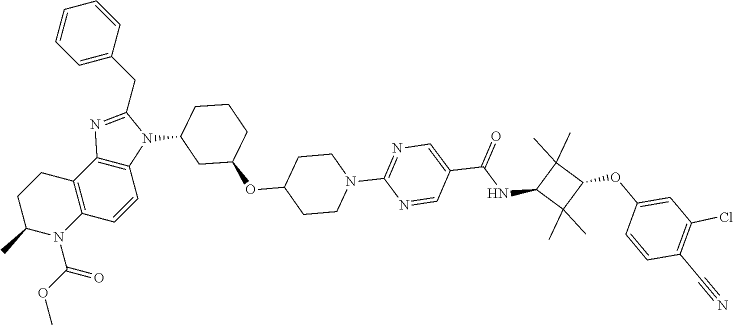 [SMILES: COC(=O)N1c2ccc3c(nc(Cc4ccccc4)n3[C@@H]3CCC[C@@H](OC4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 126 | 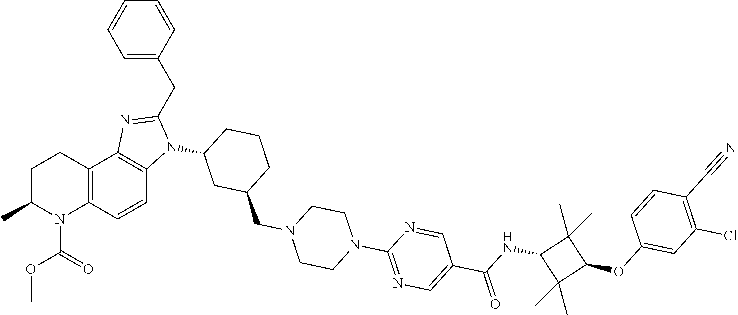 [SMILES: COC(=O)N1c2ccc3c(nc(Cc4ccccc4)n3[C@@H]3CCC[C@@H](CN4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 127 | 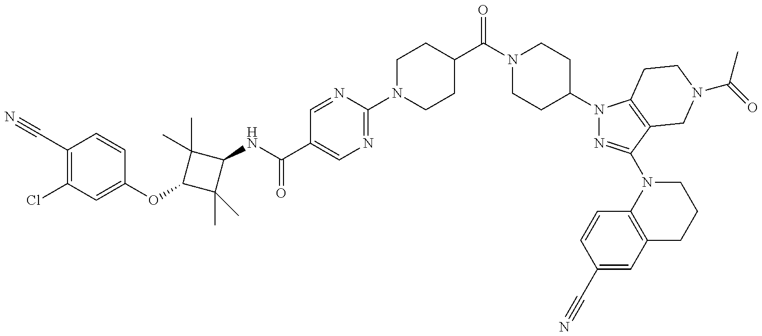 [SMILES: CC(=O)N1CCc2c(c(N3CCCc4cc(C#N)ccc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1] |
| 128 | 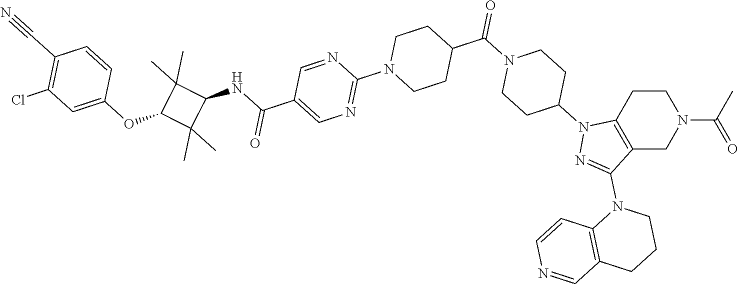 [SMILES: CC(=O)N1CCc2c(c(N3CCCc4cnccc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1] |
| 129 | 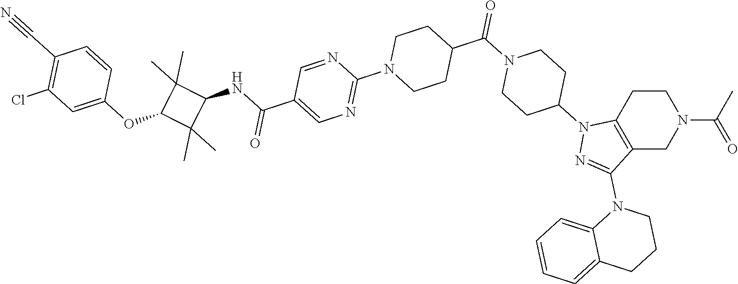 [SMILES: CC(=O)N1CCc2c(c(N3CCCc4ccccc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1] |
| 130 | 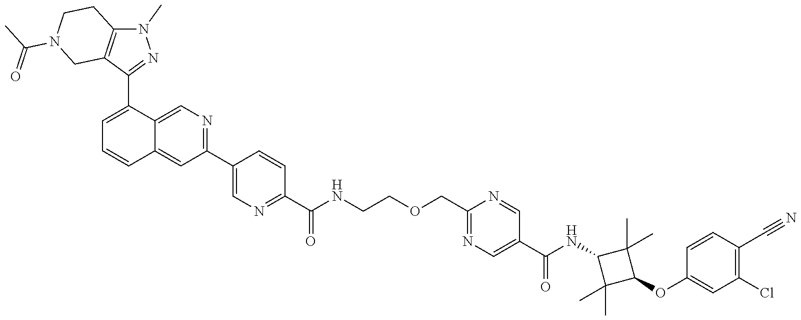 [SMILES: CC(=O)N1CCc2c(c(-c3cccc4cc(-c5ccc(C(=O)NCCOCc6ncc(C(=O)N[C@H]7C(C)(C)[C@H](Oc8ccc(C#N)c(Cl)c8)C7(C)C)cn6)nc5)ncc34)nn2C)C1] |
| 131 | 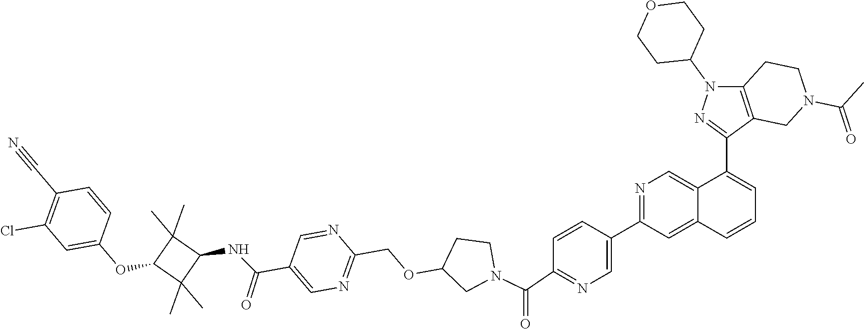 [SMILES: CC(=O)N1CCc2c(c(-c3cccc4cc(-c5ccc(C(=O)N6CCC(OCc7ncc(C(=O)N[C@H]8C(C)(C)[C@H](Oc9ccc(C#N)c(Cl)c9)C8(C)C)cn7)C6)nc5)ncc34)nn2C2CCOCC2)C1] |
| 132 | 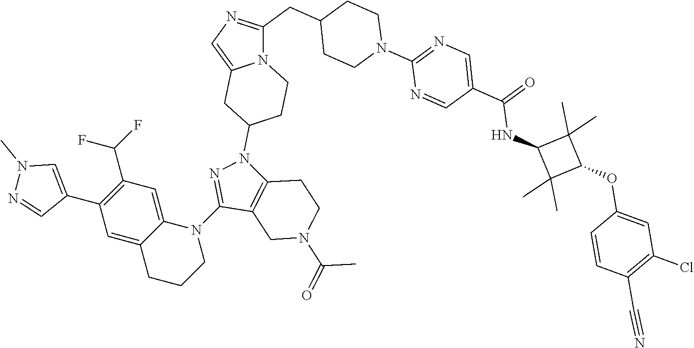 [SMILES: CC(=O)N1CCc2c(c(N3CCCc4cc(-c5cnn(C)c5)c(C(F)F)cc43)nn2C2CCn3c(cnc3CC3CCN(c4ncc(C(=O)N[C@H]5C(C)(C)[C@H](Oc6ccc(C#N)c(Cl)c6)C5(C)C)cn4)CC3)C2)C1] |
| 133 | 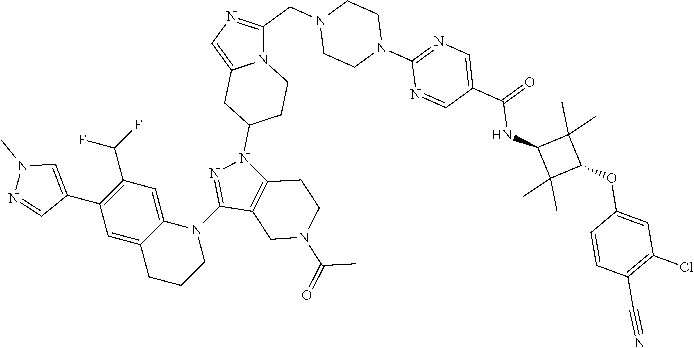 [SMILES: CC(=O)N1CCc2c(c(N3CCCc4cc(-c5cnn(C)c5)c(C(F)F)cc43)nn2C2CCn3c(cnc3CN3CCN(c4ncc(C(=O)N[C@H]5C(C)(C)[C@H](Oc6ccc(C#N)c(Cl)c6)C5(C)C)cn4)CC3)C2)C1] |
| 134 | 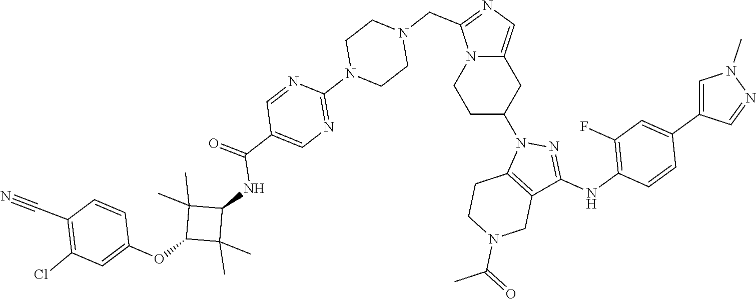 [SMILES: CC(=O)N1CCc2c(c(Nc3ccc(-c4cnn(C)c4)cc3F)nn2C2CCn3c(cnc3CN3CCN(c4ncc(C(=O)N[C@H]5C(C)(C)[C@H](Oc6ccc(C#N)c(Cl)c6)C5(C)C)cn4)CC3)C2)C1] |
| 135 | 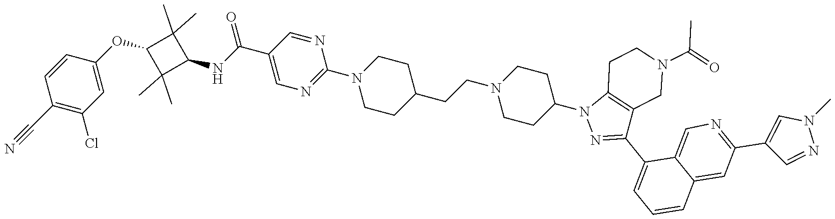 [SMILES: CC(=O)N1CCc2c(c(-c3cccc4cc(-c5cnn(C)c5)ncc34)nn2C2CCN(CCC3CCN(c4ncc(C(=O)N[C@H]5C(C)(C)[C@H](Oc6ccc(C#N)c(Cl)c6)C5(C)C)cn4)CC3)CC2)C1] |
| 136 | 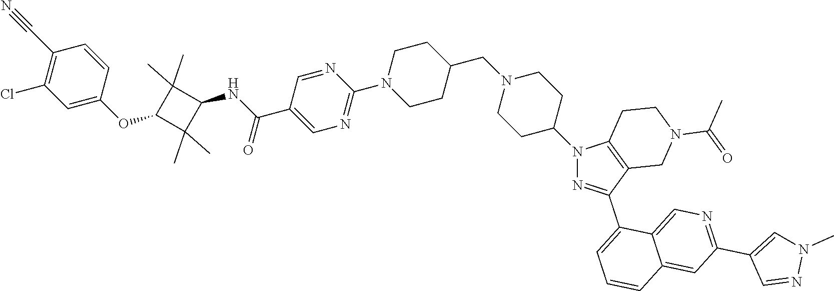 [SMILES: CC(=O)N1CCc2c(c(-c3cccc4cc(-c5cnn(C)c5)ncc34)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 137 | 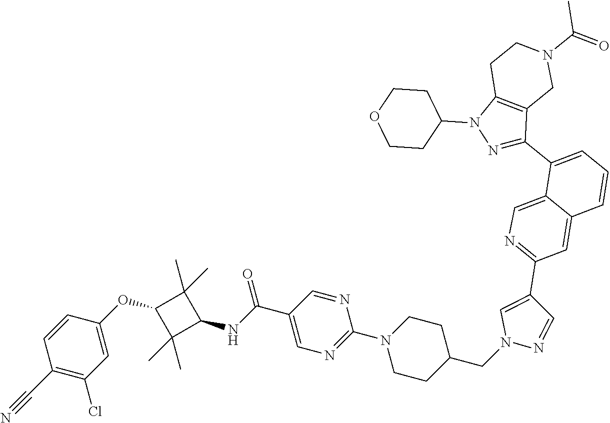 [SMILES: CC(=O)N1CCc2c(c(-c3cccc4cc(-c5cnn(CC6CCN(c7ncc(C(=O)N[C@H]8C(C)(C)[C@H](Oc9ccc(C#N)c(Cl)c9)C8(C)C)cn7)CC6)c5)ncc34)nn2C2CCOCC2)C1] |
| 138 | 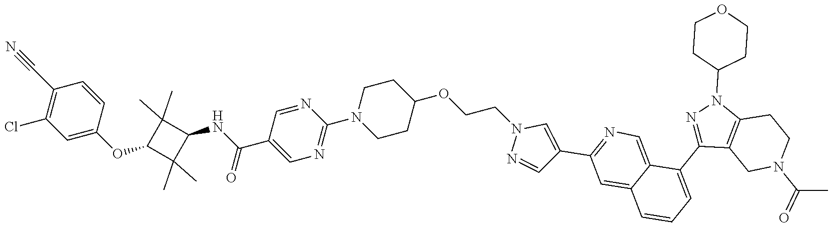 [SMILES: CC(=O)N1CCc2c(c(-c3cccc4cc(-c5cnn(CCOC6CCN(c7ncc(C(=O)N[C@H]8C(C)(C)[C@H](Oc9ccc(C#N)c(Cl)c9)C8(C)C)cn7)CC6)c5)ncc34)nn2C2CCOCC2)C1] |
| 139 | 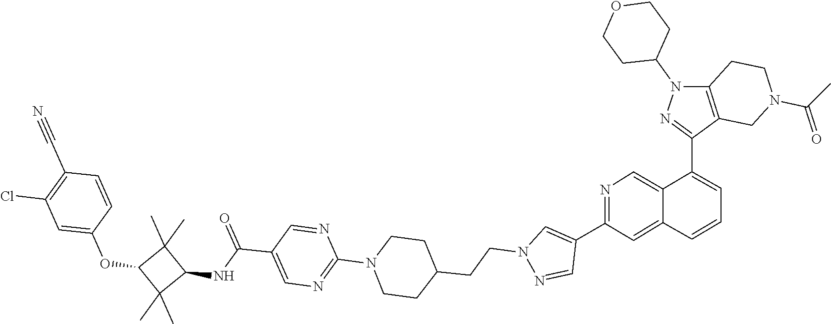 [SMILES: CC(=O)N1CCc2c(c(-c3cccc4cc(-c5cnn(CCC6CCN(c7ncc(C(=O)N[C@H]8C(C)(C)[C@H](Oc9ccc(C#N)c(Cl)c9)C8(C)C)cn7)CC6)c5)ncc34)nn2C2CCOCC2)C1] |
| 140 | 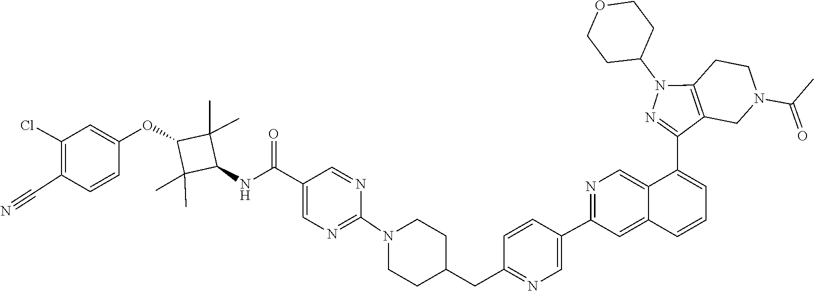 [SMILES: CC(=O)N1CCc2c(c(-c3cccc4cc(-c5ccc(CC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 141 | 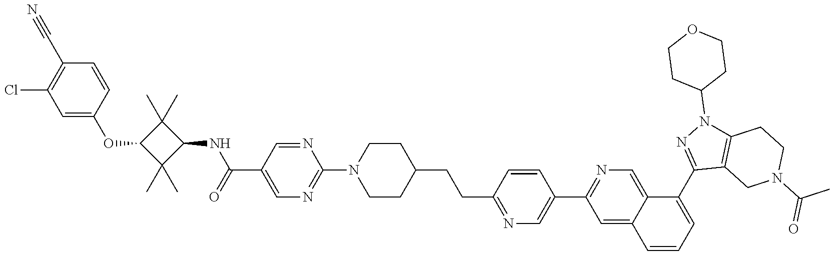 [SMILES: CC(=O)N1CCc2c(c(-c3cccc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 142 | 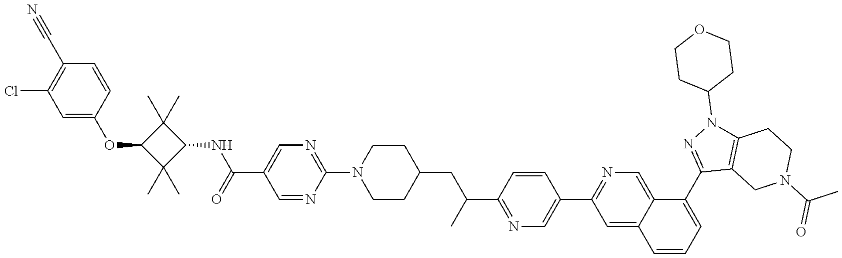 [SMILES: CC(=O)N1CCc2c(c(-c3cccc4cc(-c5ccc(C(C)CC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 143 | 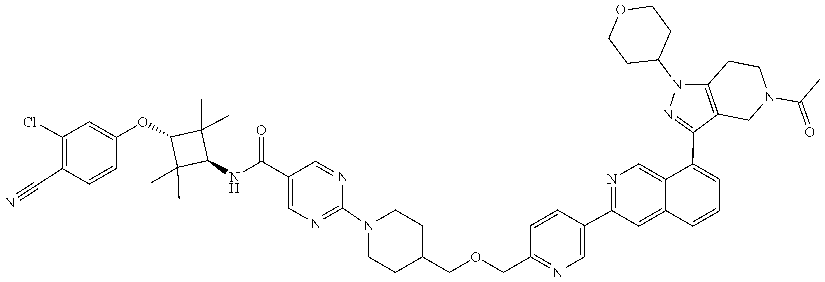 [SMILES: CC(=O)N1CCc2c(c(-c3cccc4cc(-c5ccc(COCC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 144 | 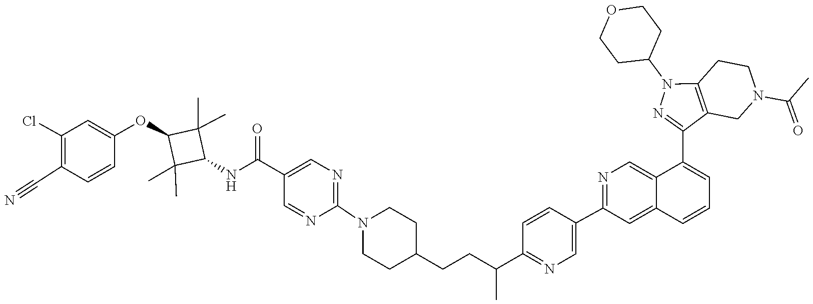 [SMILES: CC(=O)N1CCc2c(c(-c3cccc4cc(-c5ccc(C(C)CCC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 145 | 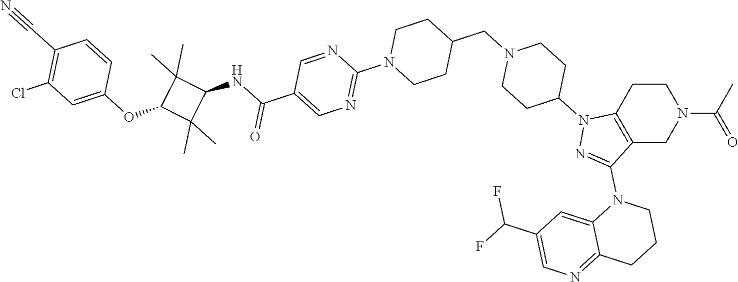 [SMILES: CC(=O)N1CCc2c(c(N3CCCc4ncc(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 146 | 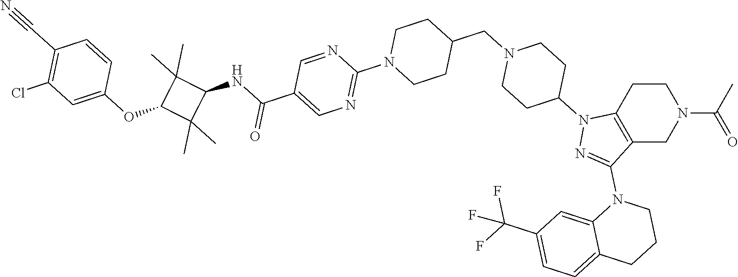 [SMILES: CC(=O)N1CCc2c(c(N3CCCc4ccc(C(F)(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 147 | 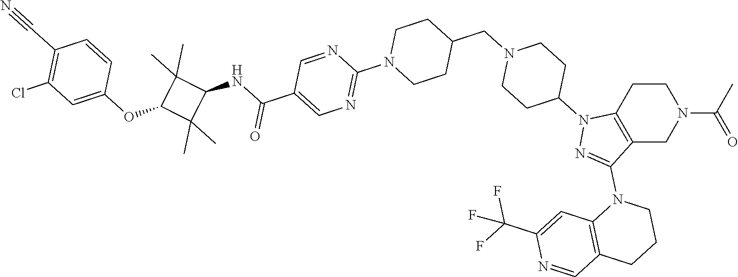 [SMILES: CC(=O)N1CCc2c(c(N3CCCc4cnc(C(F)(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 148 | 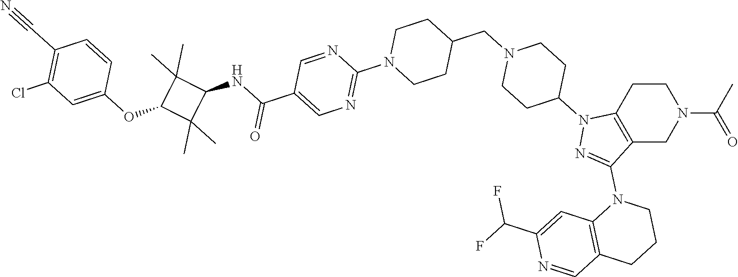 [SMILES: CC(=O)N1CCc2c(c(N3CCCc4cnc(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 149 | 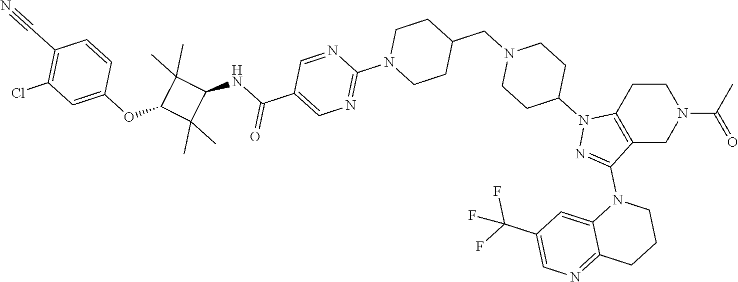 [SMILES: CC(=O)N1CCc2c(c(N3CCCc4ncc(C(F)(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 150 | 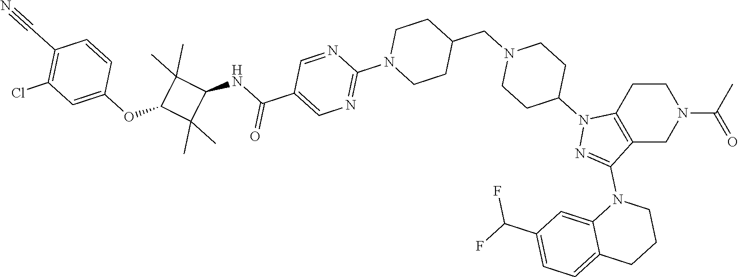 [SMILES: CC(=O)N1CCc2c(c(N3CCCc4ccc(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 151 | 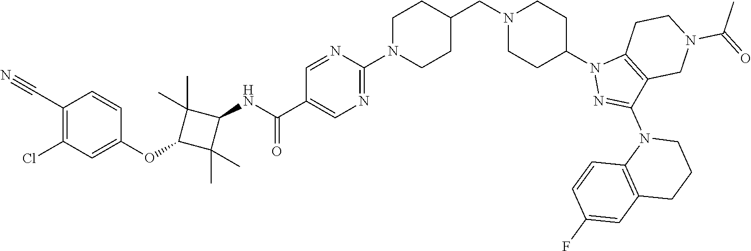 [SMILES: CC(=O)N1CCc2c(c(N3CCCc4cc(F)ccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 152 | 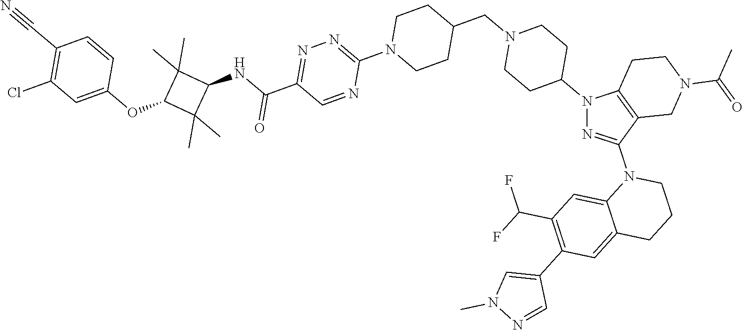 [SMILES: CC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)nn4)CC3)CC2)C1] |
| 153 | 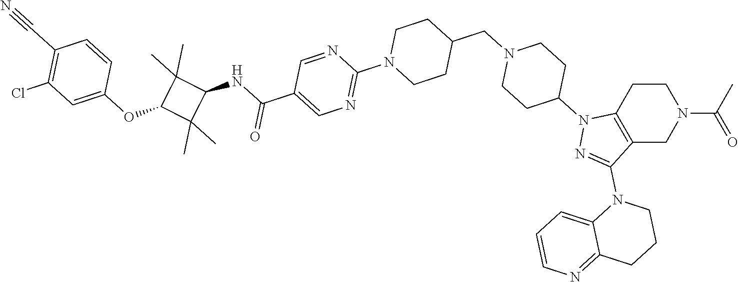 [SMILES: CC(=O)N1CCc2c(c(N3CCCc4ncccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 154 | 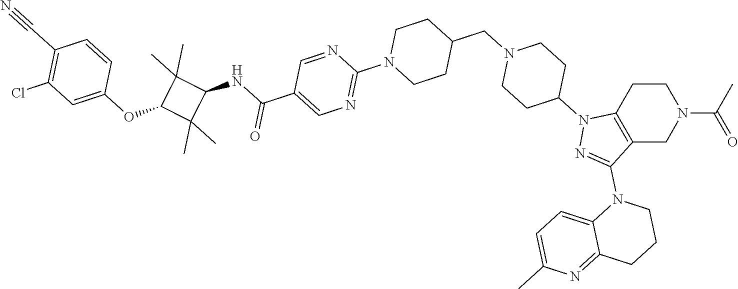 [SMILES: CC(=O)N1CCc2c(c(N3CCCc4nc(C)ccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 155 | 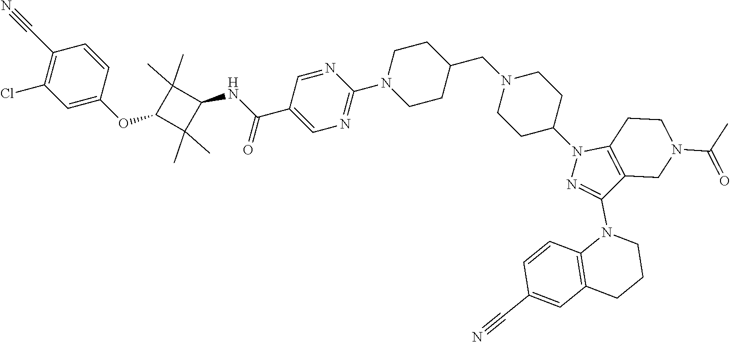 [SMILES: CC(=O)N1CCc2c(c(N3CCCc4cc(C#N)ccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 156 | 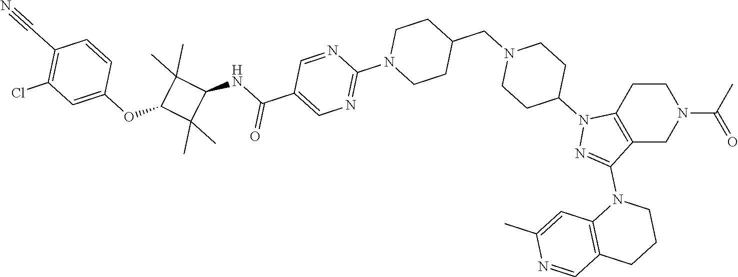 [SMILES: CC(=O)N1CCc2c(c(N3CCCc4cnc(C)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 157 | 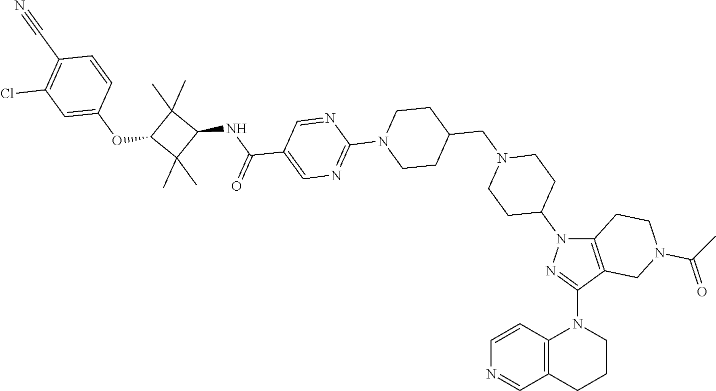 [SMILES: CC(=O)N1CCc2c(c(N3CCCc4cnccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 158 | 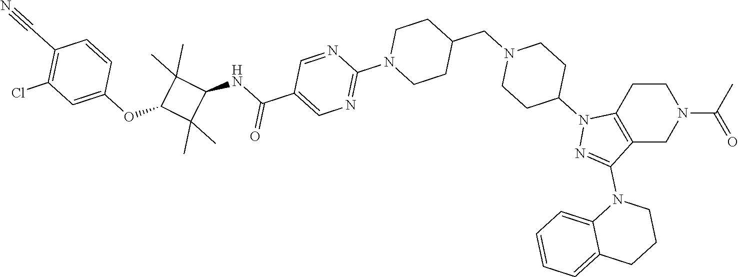 [SMILES: CC(=O)N1CCc2c(c(N3CCCc4ccccc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 159 | 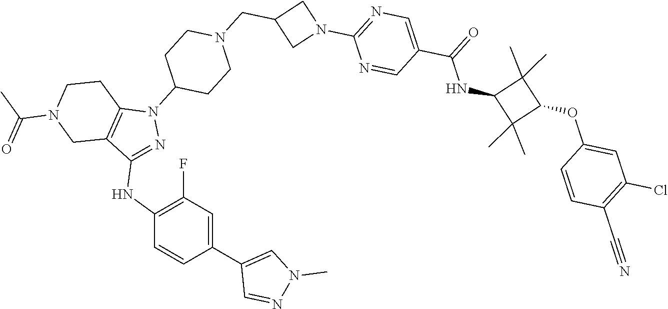 [SMILES: CC(=O)N1CCc2c(c(Nc3ccc(-c4cnn(C)c4)cc3F)nn2C2CCN(CC3CN(c4ncc(C(=O)N[C@H]5C(C)(C)[C@H](Oc6ccc(C#N)c(Cl)c6)C5(C)C)cn4)C3)CC2)C1] |
| 160 | 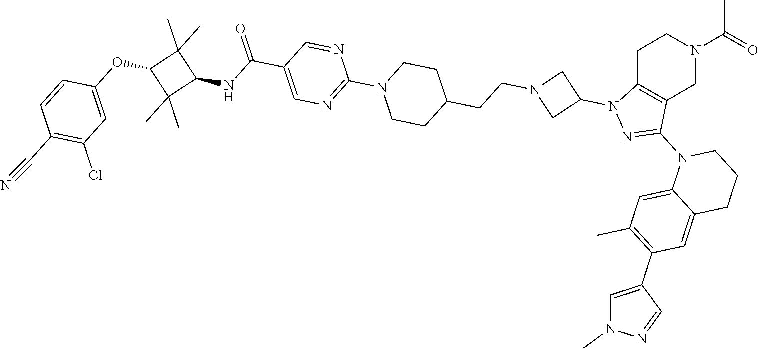 [SMILES: CC(=O)N1CCc2c(c(N3CCCc4cc(-c5cnn(C)c5)c(C)cc43)nn2C2CN(CCC3CCN(c4ncc(C(=O)N[C@H]5C(C)(C)[C@H](Oc6ccc(C#N)c(Cl)c6)C5(C)C)cn4)CC3)C2)C1] |
| 161 | 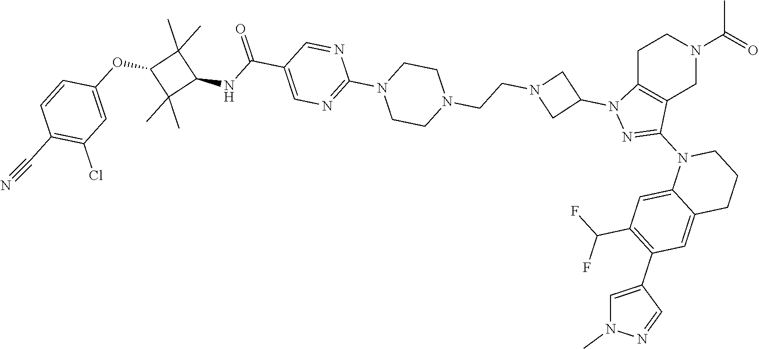 [SMILES: CC(=O)N1CCc2c(c(N3CCCc4cc(-c5cnn(C)c5)c(C(F)F)cc43)nn2C2CN(CCN3CCN(c4ncc(C(=O)N[C@H]5C(C)(C)[C@H](Oc6ccc(C#N)c(Cl)c6)C5(C)C)cn4)CC3)C2)C1] |
| 162 | 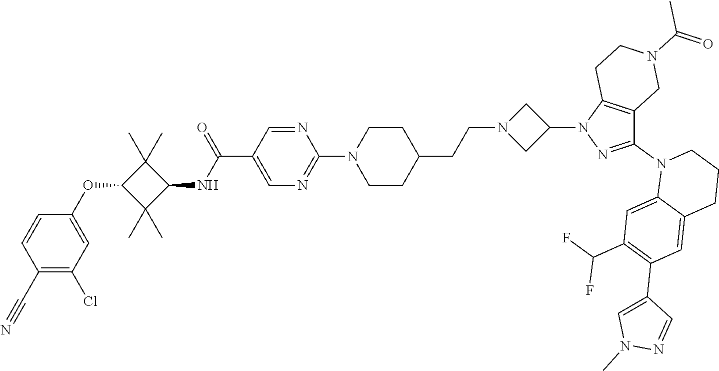 [SMILES: CC(=O)N1CCc2c(c(N3CCCc4cc(-c5cnn(C)c5)c(C(F)F)cc43)nn2C2CN(CCC3CCN(c4ncc(C(=O)N[C@H]5C(C)(C)[C@H](Oc6ccc(C#N)c(Cl)c6)C5(C)C)cn4)CC3)C2)C1] |
| 163 | 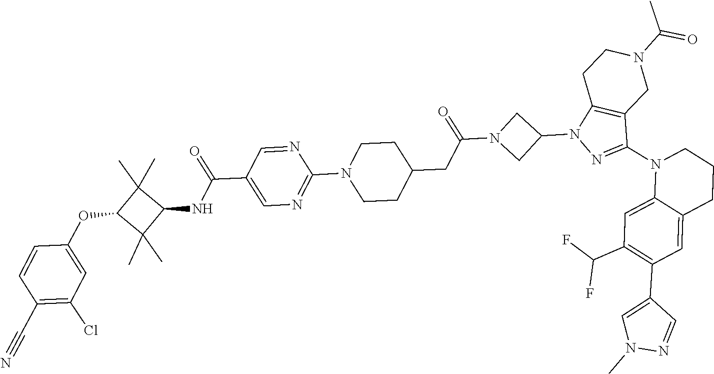 [SMILES: CC(=O)N1CCc2c(c(N3CCCc4cc(-c5cnn(C)c5)c(C(F)F)cc43)nn2C2CN(C(=O)CC3CCN(c4ncc(C(=O)N[C@H]5C(C)(C)[C@H](Oc6ccc(C#N)c(Cl)c6)C5(C)C)cn4)CC3)C2)C1] |
| 164 | 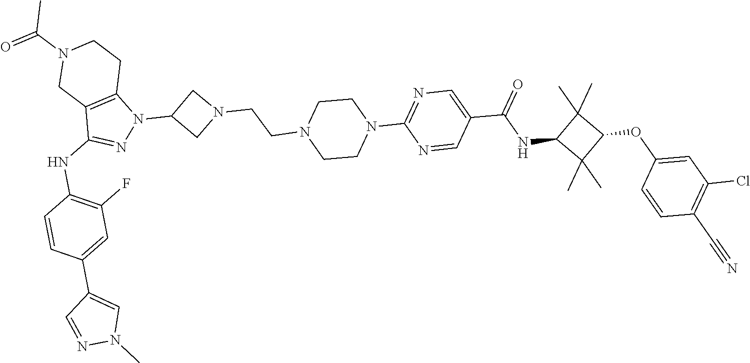 [SMILES: CC(=O)N1CCc2c(c(Nc3ccc(-c4cnn(C)c4)cc3F)nn2C2CN(CCN3CCN(c4ncc(C(=O)N[C@H]5C(C)(C)[C@H](Oc6ccc(C#N)c(Cl)c6)C5(C)C)cn4)CC3)C2)C1] |
| 165 | 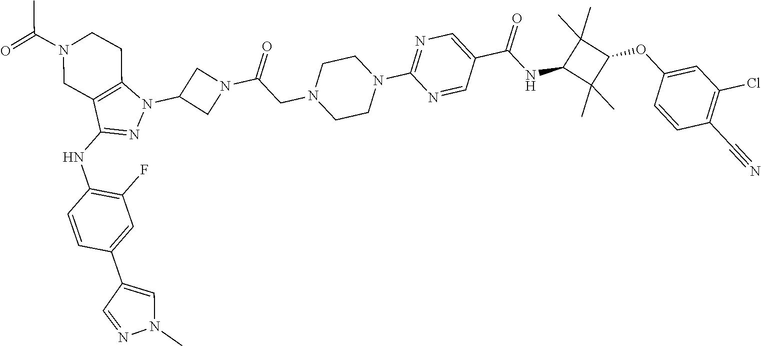 [SMILES: CC(=O)N1CCc2c(c(Nc3ccc(-c4cnn(C)c4)cc3F)nn2C2CN(C(=O)CN3CCN(c4ncc(C(=O)N[C@H]5C(C)(C)[C@H](Oc6ccc(C#N)c(Cl)c6)C5(C)C)cn4)CC3)C2)C1] |
| 166 | 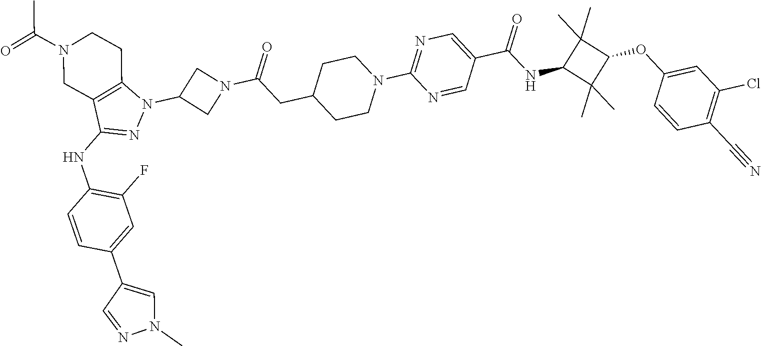 [SMILES: CC(=O)N1CCc2c(c(Nc3ccc(-c4cnn(C)c4)cc3F)nn2C2CN(C(=O)CC3CCN(c4ncc(C(=O)N[C@H]5C(C)(C)[C@H](Oc6ccc(C#N)c(Cl)c6)C5(C)C)cn4)CC3)C2)C1] |
| 167 | 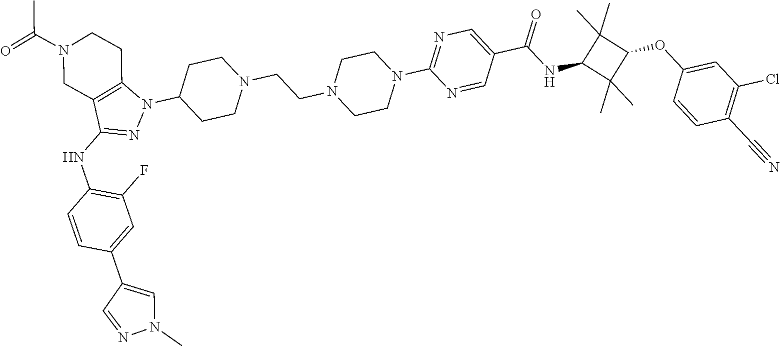 [SMILES: CC(=O)N1CCc2c(c(Nc3ccc(-c4cnn(C)c4)cc3F)nn2C2CCN(CCN3CCN(c4ncc(C(=O)N[C@H]5C(C)(C)[C@H](Oc6ccc(C#N)c(Cl)c6)C5(C)C)cn4)CC3)CC2)C1] |
| 168 | 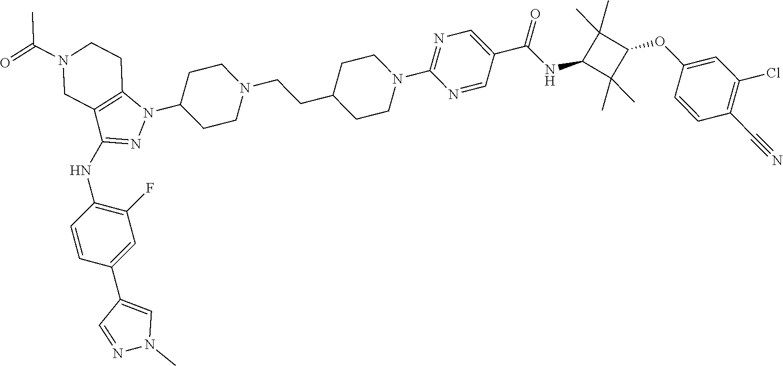 [SMILES: CC(=O)N1CCc2c(c(Nc3ccc(-c4cnn(C)c4)cc3F)nn2C2CCN(CCC3CCN(c4ncc(C(=O)N[C@H]5C(C)(C)[C@H](Oc6ccc(C#N)c(Cl)c6)C5(C)C)cn4)CC3)CC2)C1] |
| 169 | 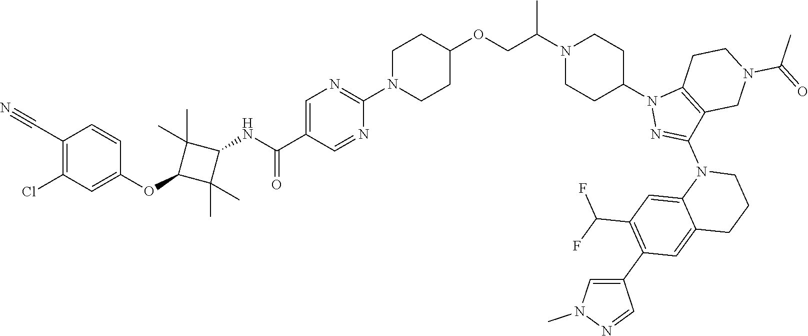 [SMILES: CC(=O)N1CCc2c(c(N3CCCc4cc(-c5cnn(C)c5)c(C(F)F)cc43)nn2C2CCN(C(C)COC3CCN(c4ncc(C(=O)N[C@H]5C(C)(C)[C@H](Oc6ccc(C#N)c(Cl)c6)C5(C)C)cn4)CC3)CC2)C1] |
| 170 | 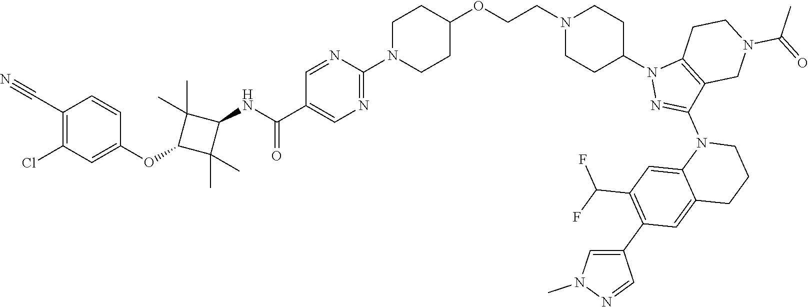 [SMILES: CC(=O)N1CCc2c(c(N3CCCc4cc(-c5cnn(C)c5)c(C(F)F)cc43)nn2C2CCN(CCOC3CCN(c4ncc(C(=O)N[C@H]5C(C)(C)[C@H](Oc6ccc(C#N)c(Cl)c6)C5(C)C)cn4)CC3)CC2)C1] |
| 171 | 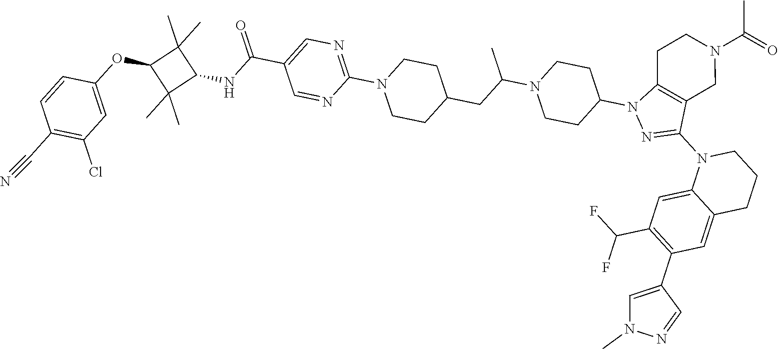 [SMILES: CC(=O)N1CCc2c(c(N3CCCc4cc(-c5cnn(C)c5)c(C(F)F)cc43)nn2C2CCN(C(C)CC3CCN(c4ncc(C(=O)N[C@H]5C(C)(C)[C@H](Oc6ccc(C#N)c(Cl)c6)C5(C)C)cn4)CC3)CC2)C1] |
| 172 | 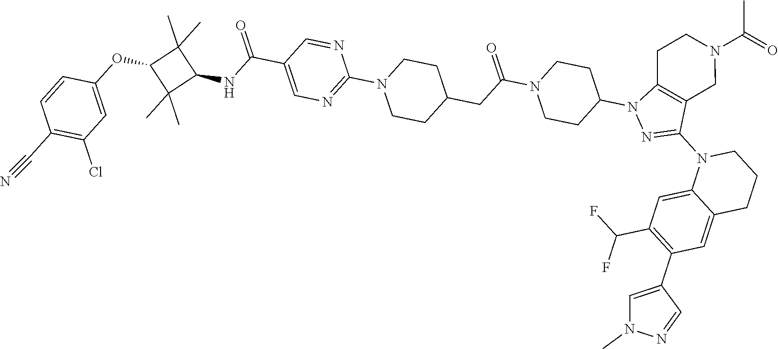 [SMILES: CC(=O)N1CCc2c(c(N3CCCc4cc(-c5cnn(C)c5)c(C(F)F)cc43)nn2C2CCN(C(=O)CC3CCN(c4ncc(C(=O)N[C@H]5C(C)(C)[C@H](Oc6ccc(C#N)c(Cl)c6)C5(C)C)cn4)CC3)CC2)C1] |
| 173 | 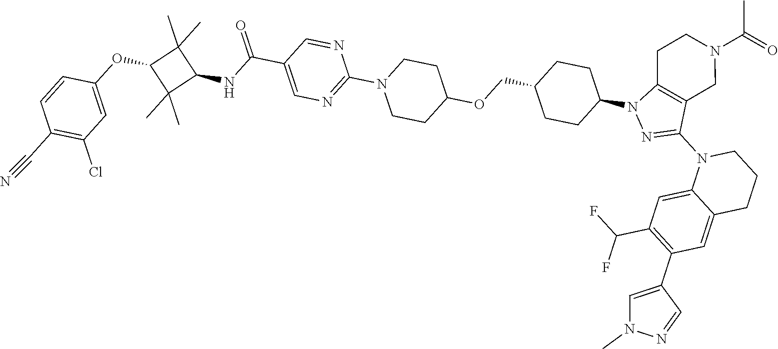 [SMILES: CC(=O)N1CCc2c(c(N3CCCc4cc(-c5cnn(C)c5)c(C(F)F)cc43)nn2[C@H]2CC[C@H](COC3CCN(c4ncc(C(=O)N[C@H]5C(C)(C)[C@H](Oc6ccc(C#N)c(Cl)c6)C5(C)C)cn4)CC3)CC2)C1] |
| 174 | 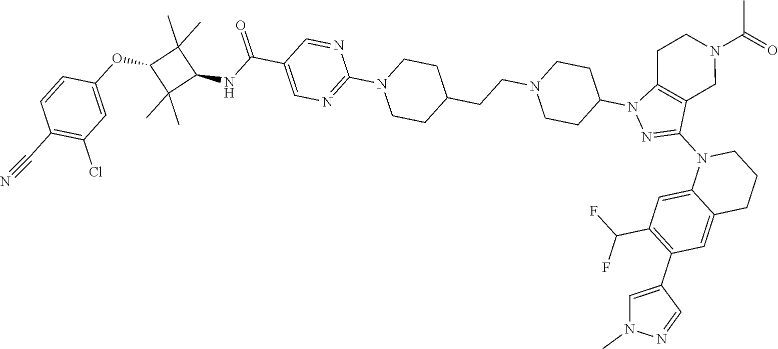 [SMILES: CC(=O)N1CCc2c(c(N3CCCc4cc(-c5cnn(C)c5)c(C(F)F)cc43)nn2C2CCN(CCC3CCN(c4ncc(C(=O)N[C@H]5C(C)(C)[C@H](Oc6ccc(C#N)c(Cl)c6)C5(C)C)cn4)CC3)CC2)C1] |
| 175 | 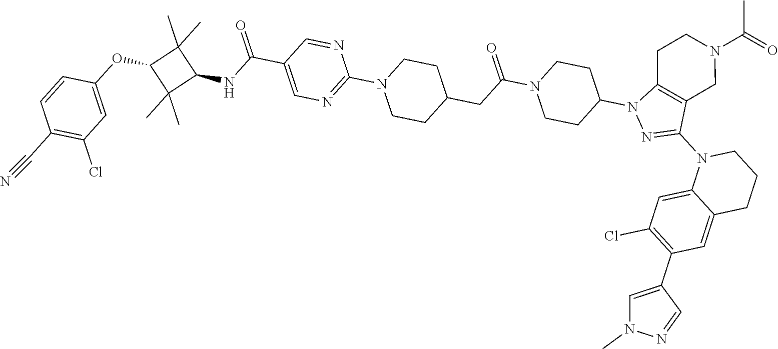 [SMILES: CC(=O)N1CCc2c(c(N3CCCc4cc(-c5cnn(C)c5)c(Cl)cc43)nn2C2CCN(C(=O)CC3CCN(c4ncc(C(=O)N[C@H]5C(C)(C)[C@H](Oc6ccc(C#N)c(Cl)c6)C5(C)C)cn4)CC3)CC2)C1] |
| 176 | 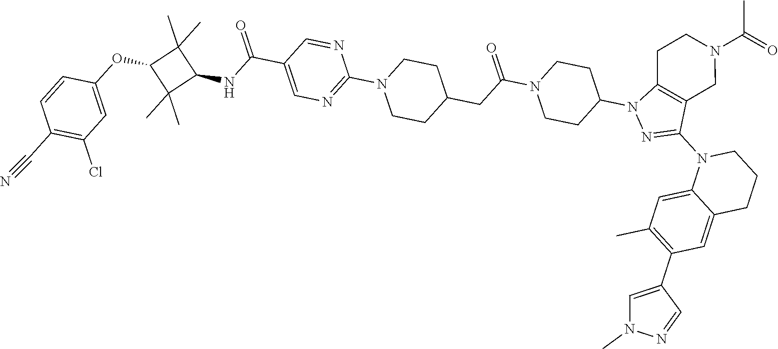 [SMILES: CC(=O)N1CCc2c(c(N3CCCc4cc(-c5cnn(C)c5)c(C)cc43)nn2C2CCN(C(=O)CC3CCN(c4ncc(C(=O)N[C@H]5C(C)(C)[C@H](Oc6ccc(C#N)c(Cl)c6)C5(C)C)cn4)CC3)CC2)C1] |
| 177 | 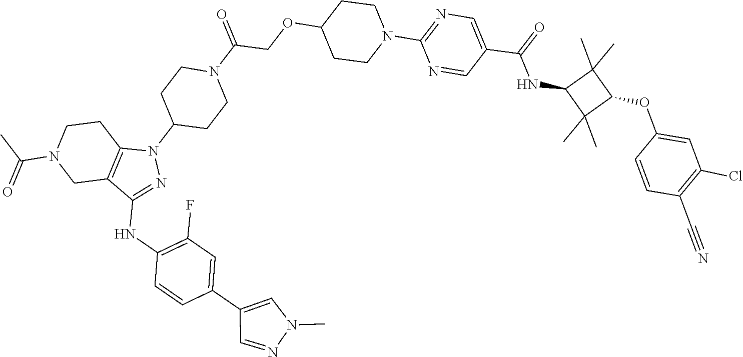 [SMILES: CC(=O)N1CCc2c(c(Nc3ccc(-c4cnn(C)c4)cc3F)nn2C2CCN(C(=O)COC3CCN(c4ncc(C(=O)N[C@H]5C(C)(C)[C@H](Oc6ccc(C#N)c(Cl)c6)C5(C)C)cn4)CC3)CC2)C1] |
| 178 | 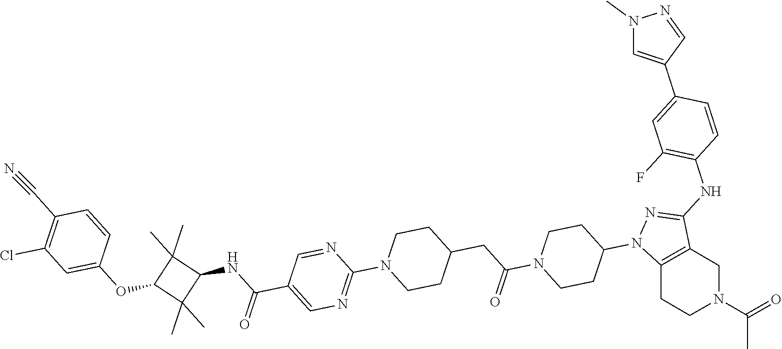 [SMILES: CC(=O)N1CCc2c(c(Nc3ccc(-c4cnn(C)c4)cc3F)nn2C2CCN(C(=O)CC3CCN(c4ncc(C(=O)N[C@H]5C(C)(C)[C@H](Oc6ccc(C#N)c(Cl)c6)C5(C)C)cn4)CC3)CC2)C1] |
| 179 | 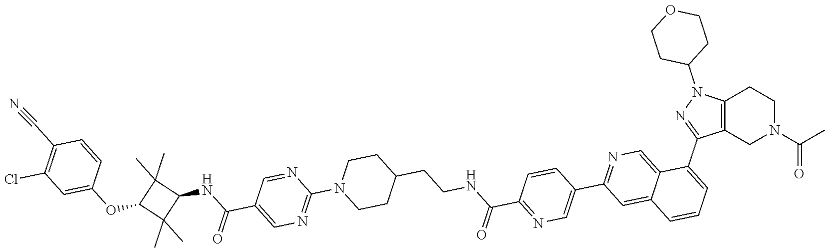 [SMILES: CC(=O)N1CCc2c(c(-c3cccc4cc(-c5ccc(C(=O)NCCC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 180 | 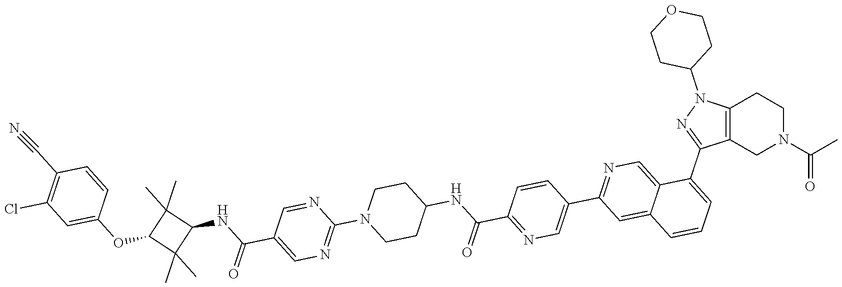 [SMILES: CC(=O)N1CCc2c(c(-c3cccc4cc(-c5ccc(C(=O)NC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1] |
| 181 | 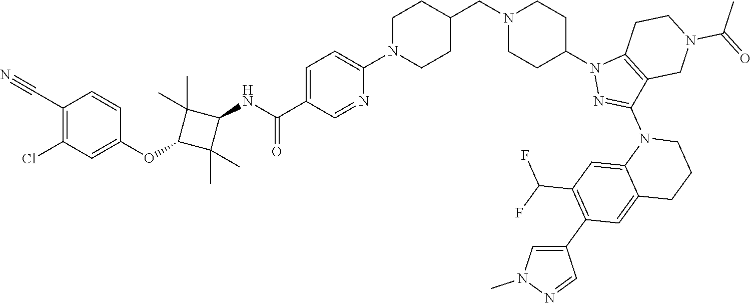 [SMILES: CC(=O)N1CCc2c(c(N3CCCc4cc(-c5cnn(C)c5)c(C(F)F)cc43)nn2C2CCN(CC3CCN(c4ccc(C(=O)N[C@H]5C(C)(C)[C@H](Oc6ccc(C#N)c(Cl)c6)C5(C)C)cn4)CC3)CC2)C1] |
| 182 | 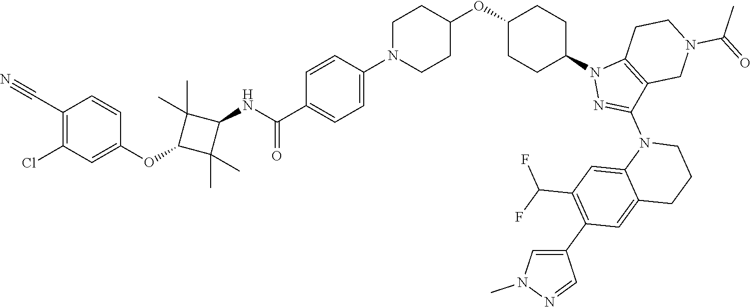 [SMILES: CC(=O)N1CCc2c(c(N3CCCc4cc(-c5cnn(C)c5)c(C(F)F)cc43)nn2[C@H]2CC[C@H](OC3CCN(c4ccc(C(=O)N[C@H]5C(C)(C)[C@H](Oc6ccc(C#N)c(Cl)c6)C5(C)C)cc4)CC3)CC2)C1] |
| 183 | 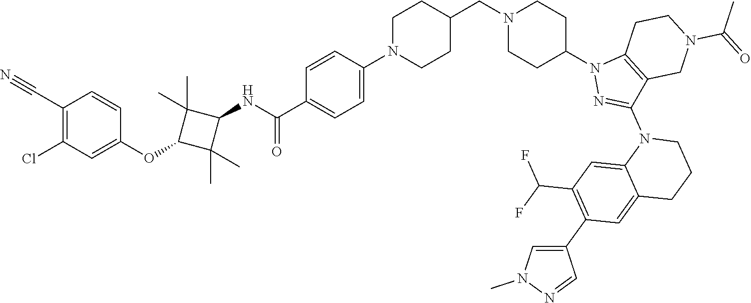 [SMILES: CC(=O)N1CCc2c(c(N3CCCc4cc(-c5cnn(C)c5)c(C(F)F)cc43)nn2C2CCN(CC3CCN(c4ccc(C(=O)N[C@H]5C(C)(C)[C@H](Oc6ccc(C#N)c(Cl)c6)C5(C)C)cc4)CC3)CC2)C1] |
| 184 | 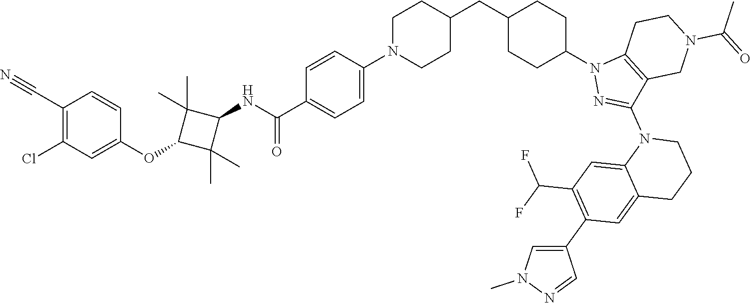 [SMILES: CC(=O)N1CCc2c(c(N3CCCc4cc(-c5cnn(C)c5)c(C(F)F)cc43)nn2C2CCC(CC3CCN(c4ccc(C(=O)N[C@H]5C(C)(C)[C@H](Oc6ccc(C#N)c(Cl)c6)C5(C)C)cc4)CC3)CC2)C1] |
| 185 | 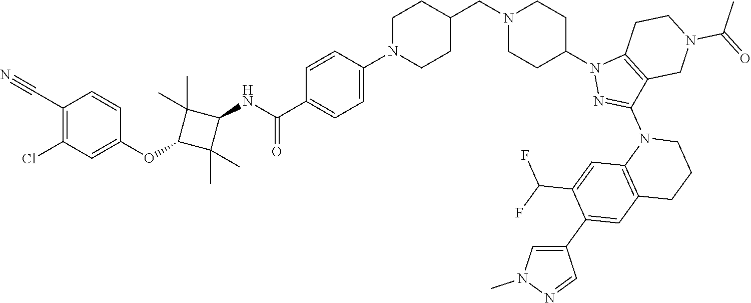 [SMILES: CC(=O)N1CCc2c(c(N3CCCc4cc(-c5cnn(C)c5)c(C(F)F)cc43)nn2C2CCN(CC3CCN(c4ccc(C(=O)N[C@H]5C(C)(C)[C@H](Oc6ccc(C#N)c(Cl)c6)C5(C)C)cc4)CC3)CC2)C1] |
| 186 | 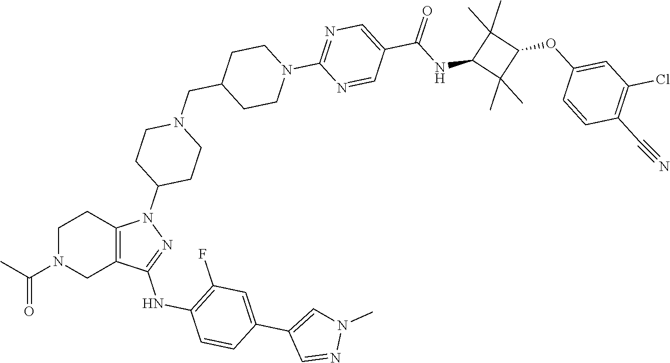 [SMILES: CC(=O)N1CCc2c(c(Nc3ccc(-c4cnn(C)c4)cc3F)nn2C2CCN(CC3CCN(c4ncc(C(=O)N[C@H]5C(C)(C)[C@H](Oc6ccc(C#N)c(Cl)c6)C5(C)C)cn4)CC3)CC2)C1] |
| 187 | 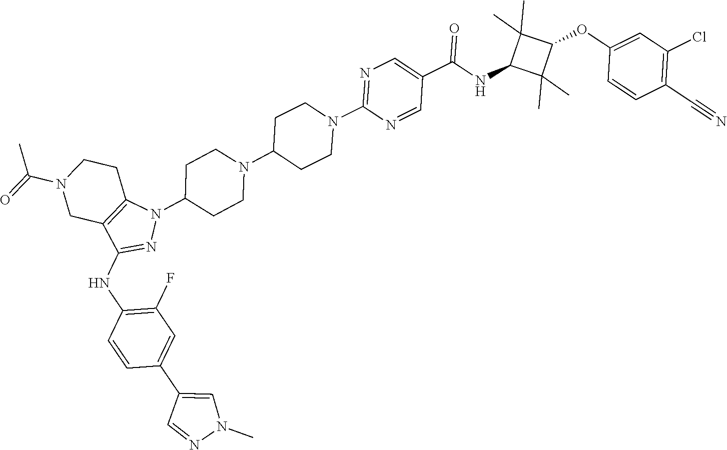 [SMILES: CC(=O)N1CCc2c(c(Nc3ccc(-c4cnn(C)c4)cc3F)nn2C2CCN(C3CCN(c4ncc(C(=O)N[C@H]5C(C)(C)[C@H](Oc6ccc(C#N)c(Cl)c6)C5(C)C)cn4)CC3)CC2)C1] |
| 188 | 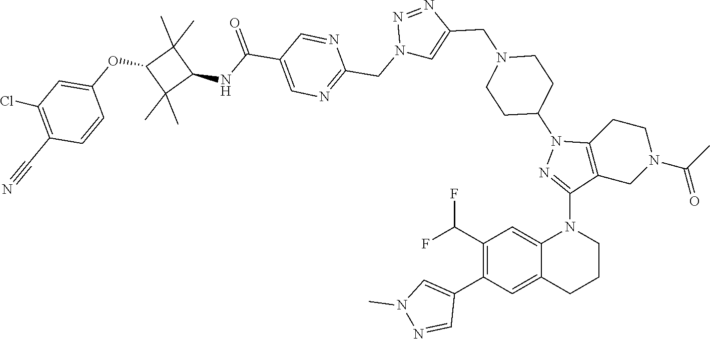 [SMILES: CC(=O)N1CCc2c(c(N3CCCc4cc(-c5cnn(C)c5)c(C(F)F)cc43)nn2C2CCN(Cc3cn(Cc4ncc(C(=O)N[C@H]5C(C)(C)[C@H](Oc6ccc(C#N)c(Cl)c6)C5(C)C)cn4)nn3)CC2)C1] |
| 189 | 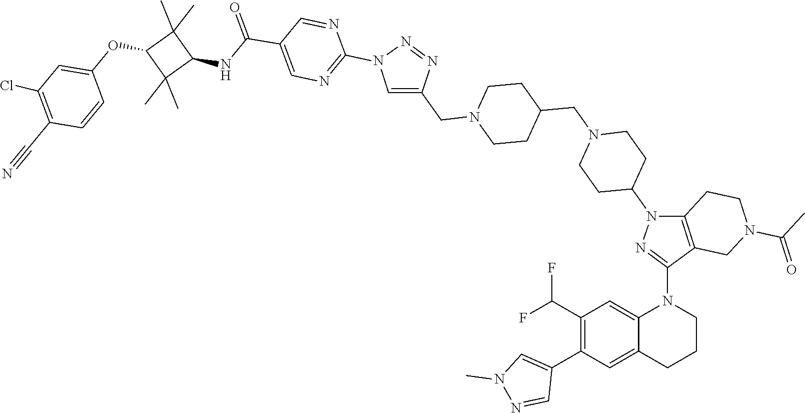 [SMILES: CC(=O)N1CCc2c(c(N3CCCc4cc(-c5cnn(C)c5)c(C(F)F)cc43)nn2C2CCN(CC3CCN(Cc4cn(-c5ncc(C(=O)N[C@H]6C(C)(C)[C@H](Oc7ccc(C#N)c(Cl)c7)C6(C)C)cn5)nn4)CC3)CC2)C1] |
| 190 | 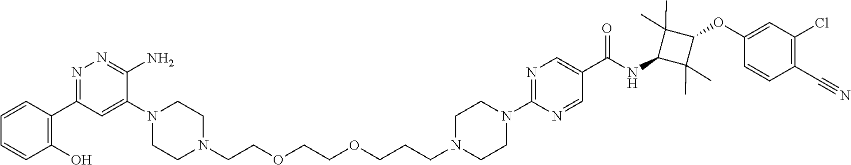 [SMILES: CC1(C)[C@H](NC(=O)c2cnc(N3CCN(CCCOCCOCCN4CCN(c5cc(-c6ccccc6O)nnc5N)CC4)CC3)nc2)C(C)(C)[C@H]1Oc1ccc(C#N)c(Cl)c1] |
| 191 | 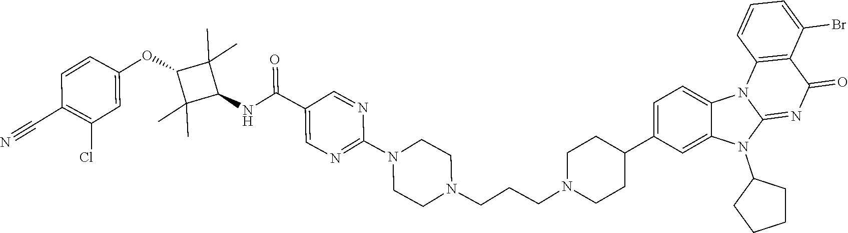 [SMILES: CC1(C)[C@H](NC(=O)c2cnc(N3CCN(CCCN4CCC(c5ccc6c(c5)n(C5CCCC5)c5nc(=O)c7c(Br)cccc7n65)CC4)CC3)nc2)C(C)(C)[C@H]1Oc1ccc(C#N)c(Cl)c1] |
| 192 | 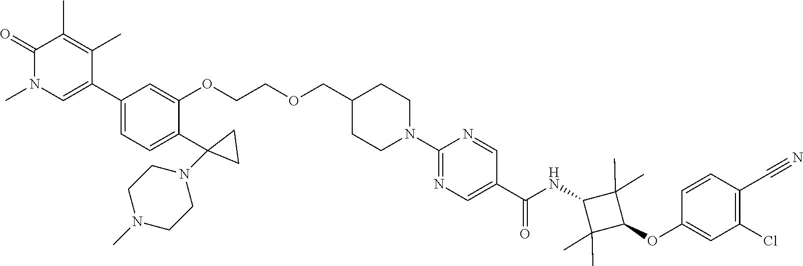 [SMILES: Cc1c(-c2ccc(C3(N4CCN(C)CC4)CC3)c(OCCOCC3CCN(c4ncc(C(=O)N[C@H]5C(C)(C)[C@H](Oc6ccc(C#N)c(Cl)c6)C5(C)C)cn4)CC3)c2)cn(C)c(=O)c1C] |
| 193 | 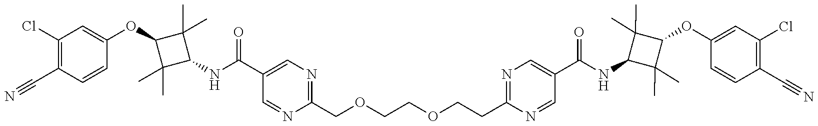 [SMILES: CC1(C)[C@H](NC(=O)c2cnc(CCOCCOCc3ncc(C(=O)N[C@H]4C(C)(C)[C@H](Oc5ccc(C#N)c(Cl)c5)C4(C)C)cn3)nc2)C(C)(C)[C@H]1Oc1ccc(C#N)c(Cl)c1] |
| 194 | 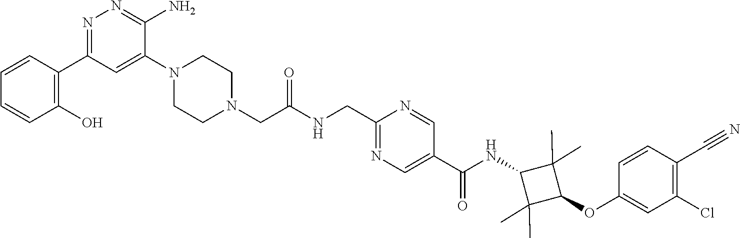 [SMILES: CC1(C)[C@H](NC(=O)c2cnc(CNC(=O)CN3CCN(c4cc(-c5ccccc5O)nnc4N)CC3)nc2)C(C)(C)[C@H]1Oc1ccc(C#N)c(Cl)c1] |
| 195 | 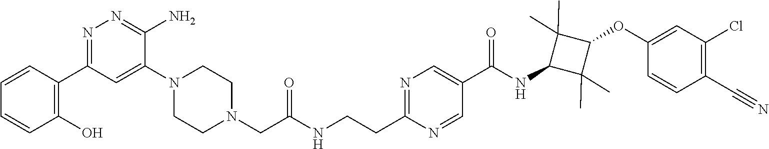 [SMILES: CC1(C)[C@H](NC(=O)c2cnc(CCNC(=O)CN3CCN(c4cc(-c5ccccc5O)nnc4N)CC3)nc2)C(C)(C)[C@H]1Oc1ccc(C#N)c(Cl)c1] |
| 196 | 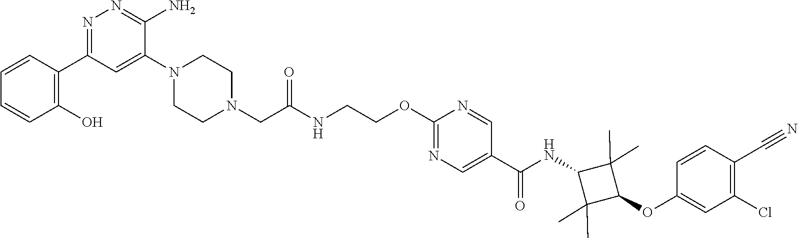 [SMILES: CC1(C)[C@H](NC(=O)c2cnc(OCCNC(=O)CN3CCN(c4cc(-c5ccccc5O)nnc4N)CC3)nc2)C(C)(C)[C@H]1Oc1ccc(C#N)c(Cl)c1] |
| 197 | 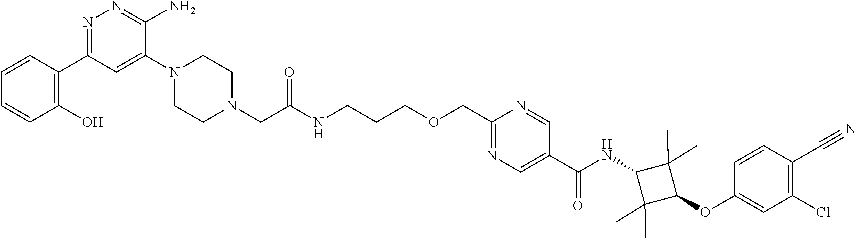 [SMILES: CC1(C)[C@H](NC(=O)c2cnc(COCCCNC(=O)CN3CCN(c4cc(-c5ccccc5O)nnc4N)CC3)nc2)C(C)(C)[C@H]1Oc1ccc(C#N)c(Cl)c1] |
| 198 | 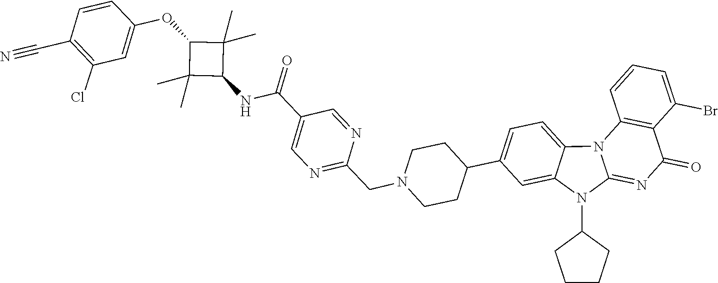 [SMILES: CC1(C)[C@H](NC(=O)c2cnc(CN3CCC(c4ccc5c(c4)n(C4CCCC4)c4nc(=O)c6c(Br)cccc6n54)CC3)nc2)C(C)(C)[C@H]1Oc1ccc(C#N)c(Cl)c1] |
| 199 | 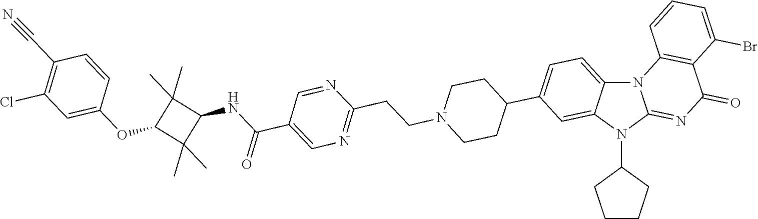 [SMILES: CC1(C)[C@H](NC(=O)c2cnc(CCN3CCC(c4ccc5c(c4)n(C4CCCC4)c4nc(=O)c6c(Br)cccc6n54)CC3)nc2)C(C)(C)[C@H]1Oc1ccc(C#N)c(Cl)c1] |
| 200 | 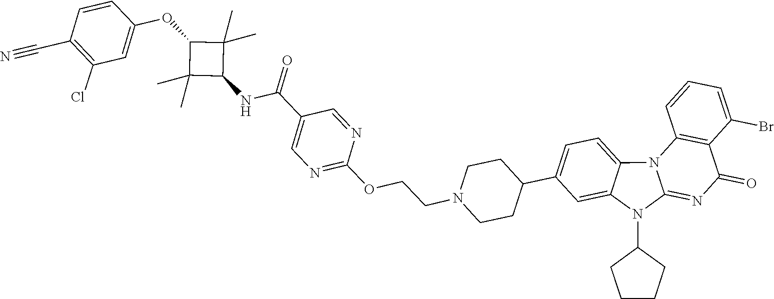 [SMILES: CC1(C)[C@H](NC(=O)c2cnc(OCCN3CCC(c4ccc5c(c4)n(C4CCCC4)c4nc(=O)c6c(Br)cccc6n54)CC3)nc2)C(C)(C)[C@H]1Oc1ccc(C#N)c(Cl)c1] |
| 201 | 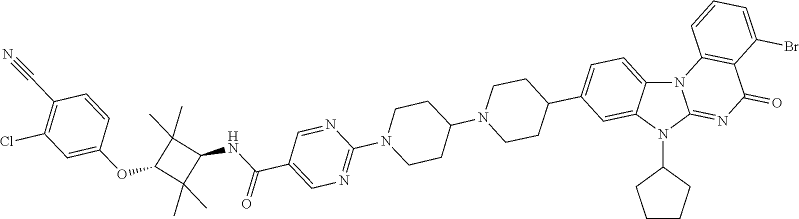 [SMILES: CC1(C)[C@H](NC(=O)c2cnc(N3CCC(N4CCC(c5ccc6c(c5)n(C5CCCC5)c5nc(=O)c7c(Br)cccc7n65)CC4)CC3)nc2)C(C)(C)[C@H]1Oc1ccc(C#N)c(Cl)c1] |
| 202 | 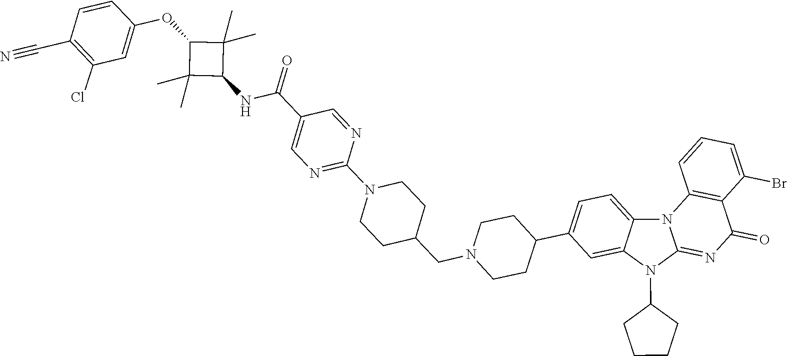 [SMILES: CC1(C)[C@H](NC(=O)c2cnc(N3CCC(CN4CCC(c5ccc6c(c5)n(C5CCCC5)c5nc(=O)c7c(Br)cccc7n65)CC4)CC3)nc2)C(C)(C)[C@H]1Oc1ccc(C#N)c(Cl)c1] |
| 203 | 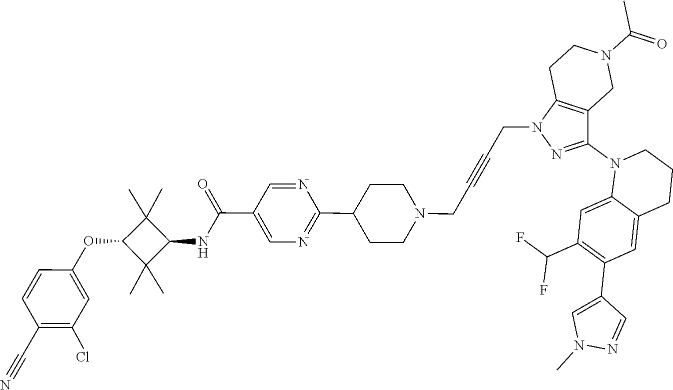 [SMILES: CC(=O)N1CCc2c(c(N3CCCc4cc(-c5cnn(C)c5)c(C(F)F)cc43)nn2CC#CCN2CCC(c3ncc(C(=O)N[C@H]4C(C)(C)[C@H](Oc5ccc(C#N)c(Cl)c5)C4(C)C)cn3)CC2)C1] |
| 204 | 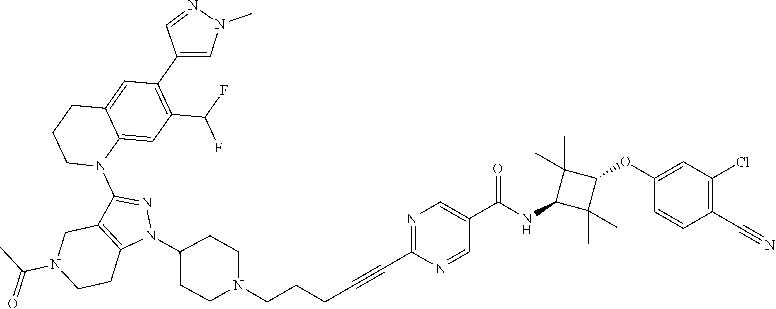 [SMILES: CC(=O)N1CCc2c(c(N3CCCc4cc(-c5cnn(C)c5)c(C(F)F)cc43)nn2C2CCN(CCCC#Cc3ncc(C(=O)N[C@H]4C(C)(C)[C@H](Oc5ccc(C#N)c(Cl)c5)C4(C)C)cn3)CC2)C1] |
| 205 | 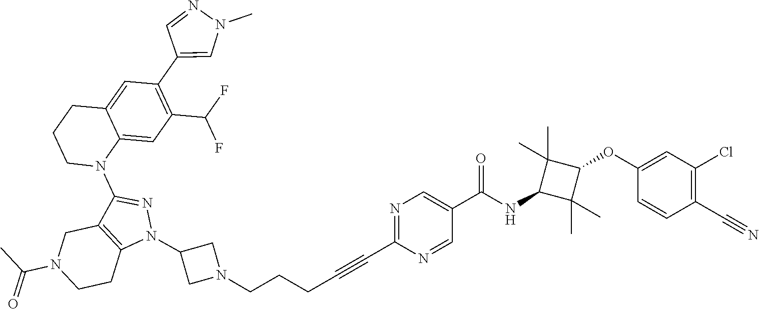 [SMILES: CC(=O)N1CCc2c(c(N3CCCc4cc(-c5cnn(C)c5)c(C(F)F)cc43)nn2C2CN(CCCC#Cc3ncc(C(=O)N[C@H]4C(C)(C)[C@H](Oc5ccc(C#N)c(Cl)c5)C4(C)C)cn3)C2)C1] |
| 206 | 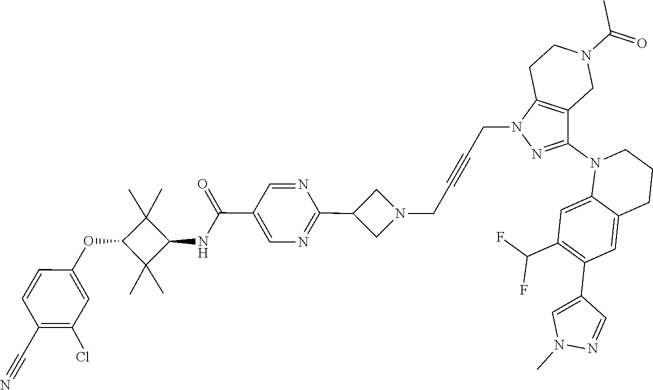 [SMILES: CC(=O)N1CCc2c(c(N3CCCc4cc(-c5cnn(C)c5)c(C(F)F)cc43)nn2CC#CCN2CC(c3ncc(C(=O)N[C@H]4C(C)(C)[C@H](Oc5ccc(C#N)c(Cl)c5)C4(C)C)cn3)C2)C1] |
| 207 | 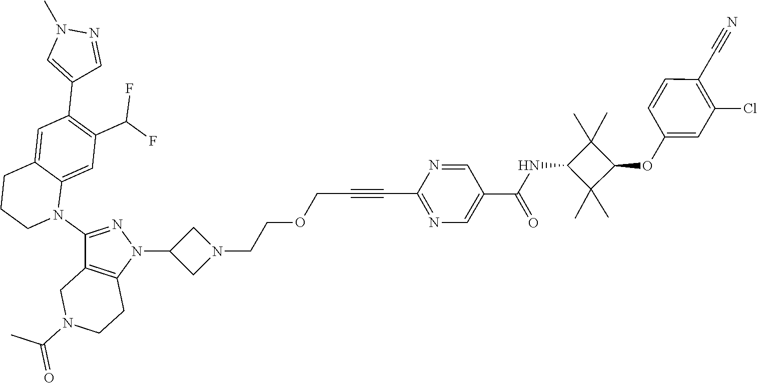 [SMILES: CC(=O)N1CCc2c(c(N3CCCc4cc(-c5cnn(C)c5)c(C(F)F)cc43)nn2C2CN(CCOCC#Cc3ncc(C(=O)N[C@H]4C(C)(C)[C@H](Oc5ccc(C#N)c(Cl)c5)C4(C)C)cn3)C2)C1] |
| 208 | 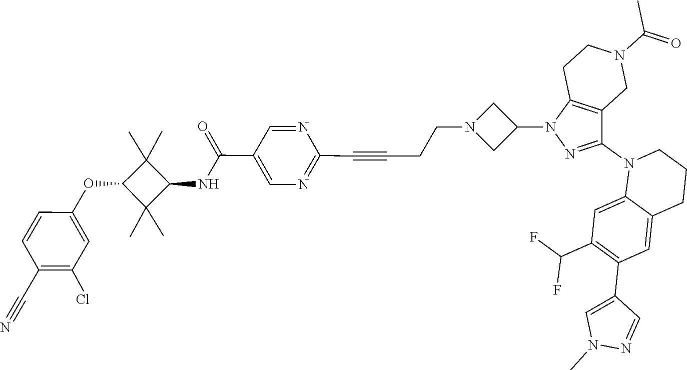 [SMILES: CC(=O)N1CCc2c(c(N3CCCc4cc(-c5cnn(C)c5)c(C(F)F)cc43)nn2C2CN(CCC#Cc3ncc(C(=O)N[C@H]4C(C)(C)[C@H](Oc5ccc(C#N)c(Cl)c5)C4(C)C)cn3)C2)C1] |
| 209 | 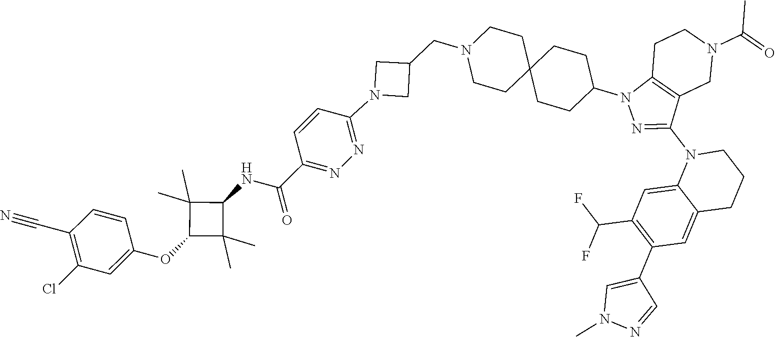 [SMILES: CC(=O)N1CCc2c(c(N3CCCc4cc(-c5cnn(C)c5)c(C(F)F)cc43)nn2C2CCC3(CC2)CCN(CC2CN(c4ccc(C(=O)N[C@H]5C(C)(C)[C@H](Oc6ccc(C#N)c(Cl)c6)C5(C)C)nn4)C2)CC3)C1] |
| 210 | 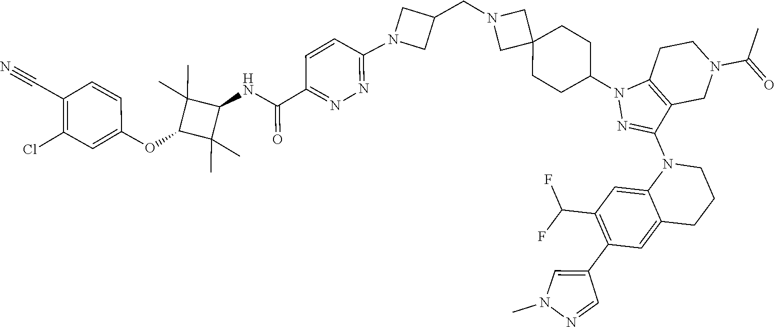 [SMILES: CC(=O)N1CCc2c(c(N3CCCc4cc(-c5cnn(C)c5)c(C(F)F)cc43)nn2C2CCC3(CC2)CN(CC2CN(c4ccc(C(=O)N[C@H]5C(C)(C)[C@H](Oc6ccc(C#N)c(Cl)c6)C5(C)C)nn4)C2)C3)C1] |
| 211 | 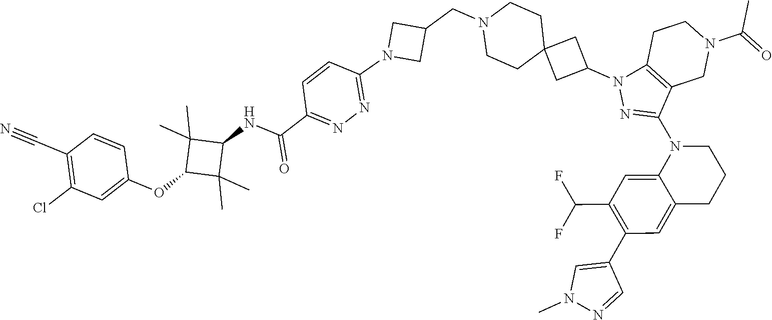 [SMILES: CC(=O)N1CCc2c(c(N3CCCc4cc(-c5cnn(C)c5)c(C(F)F)cc43)nn2C2CC3(CCN(CC4CN(c5ccc(C(=O)N[C@H]6C(C)(C)[C@H](Oc7ccc(C#N)c(Cl)c7)C6(C)C)nn5)C4)CC3)C2)C1] |
| 212 | 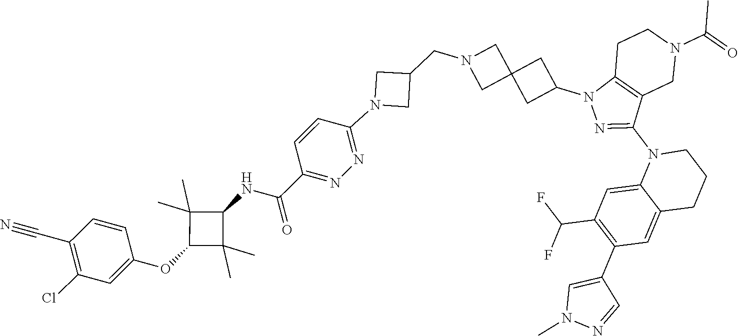 [SMILES: CC(=O)N1CCc2c(c(N3CCCc4cc(-c5cnn(C)c5)c(C(F)F)cc43)nn2C2CC3(C2)CN(CC2CN(c4ccc(C(=O)N[C@H]5C(C)(C)[C@H](Oc6ccc(C#N)c(Cl)c6)C5(C)C)nn4)C2)C3)C1] |
| 213 | 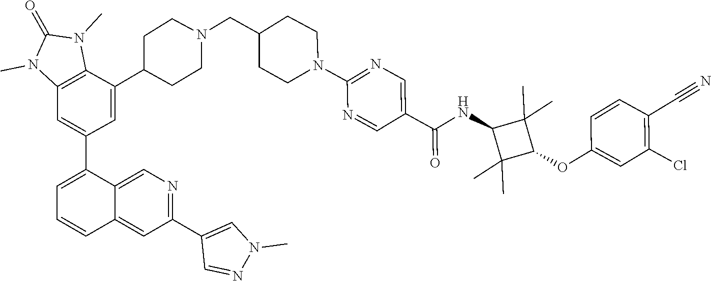 [SMILES: Cn1cc(-c2cc3cccc(-c4cc(C5CCN(CC6CCN(c7ncc(C(=O)N[C@H]8C(C)(C)[C@H](Oc9ccc(C#N)c(Cl)c9)C8(C)C)cn7)CC6)CC5)c5c(c4)n(C)c(=O)n5C)c3cn2)cn1] |
| 214 | 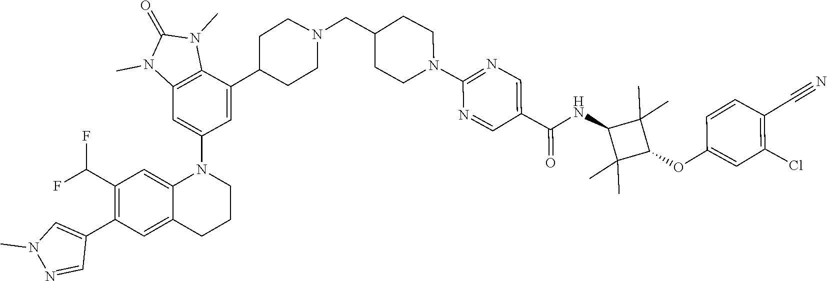 [SMILES: Cn1cc(-c2cc3c(cc2C(F)F)N(c2cc(C4CCN(CC5CCN(c6ncc(C(=O)N[C@H]7C(C)(C)[C@H](Oc8ccc(C#N)c(Cl)c8)C7(C)C)cn6)CC5)CC4)c4c(c2)n(C)c(=O)n4C)CCC3)cn1] |
| 215 | 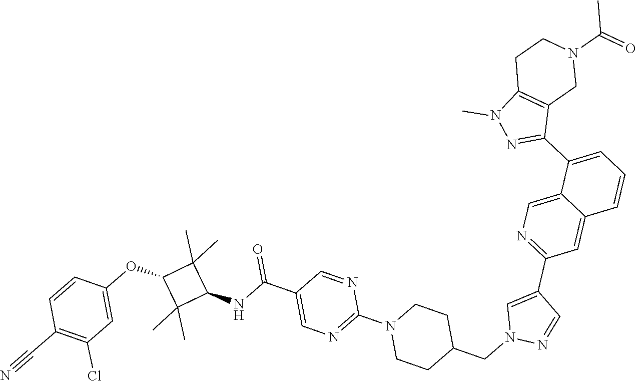 [SMILES: CC(=O)N1CCc2c(c(-c3cccc4cc(-c5cnn(CC6CCN(c7ncc(C(=O)N[C@H]8C(C)(C)[C@H](Oc9ccc(C#N)c(Cl)c9)C8(C)C)cn7)CC6)c5)ncc34)nn2C)C1] |
| 216 | 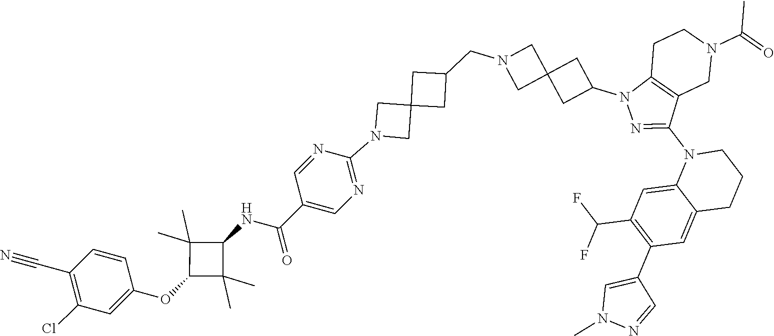 [SMILES: CC(=O)N1CCc2c(c(N3CCCc4cc(-c5cnn(C)c5)c(C(F)F)cc43)nn2C2CC3(C2)CN(CC2CC4(C2)CN(c2ncc(C(=O)N[C@H]5C(C)(C)[C@H](Oc6ccc(C#N)c(Cl)c6)C5(C)C)cn2)C4)C3)C1] |
| 217 | 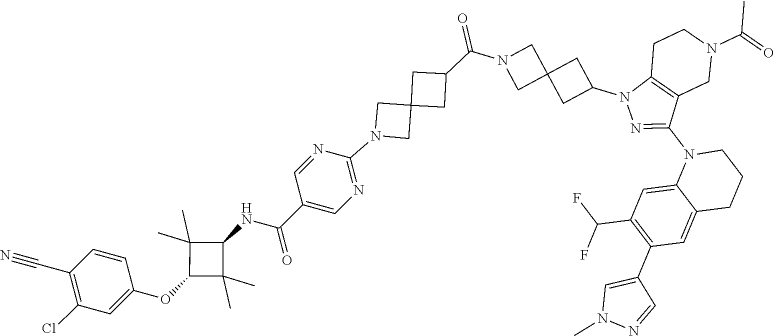 [SMILES: CC(=O)N1CCc2c(c(N3CCCc4cc(-c5cnn(C)c5)c(C(F)F)cc43)nn2C2CC3(C2)CN(C(=O)C2CC4(C2)CN(c2ncc(C(=O)N[C@H]5C(C)(C)[C@H](Oc6ccc(C#N)c(Cl)c6)C5(C)C)cn2)C4)C3)C1] |
| 218 | 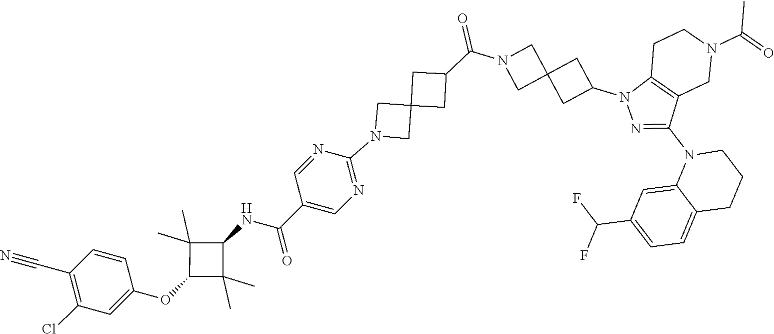 [SMILES: CC(=O)N1CCc2c(c(N3CCCc4ccc(C(F)F)cc43)nn2C2CC3(C2)CN(C(=O)C2CC4(C2)CN(c2ncc(C(=O)N[C@H]5C(C)(C)[C@H](Oc6ccc(C#N)c(Cl)c6)C5(C)C)cn2)C4)C3)C1] |
| 219 | 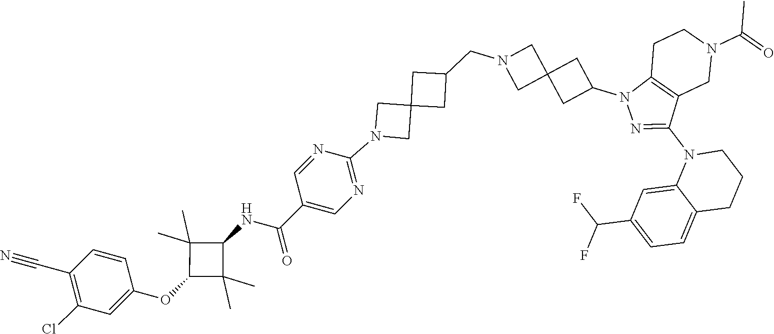 [SMILES: CC(=O)N1CCc2c(c(N3CCCc4ccc(C(F)F)cc43)nn2C2CC3(C2)CN(CC2CC4(C2)CN(c2ncc(C(=O)N[C@H]5C(C)(C)[C@H](Oc6ccc(C#N)c(Cl)c6)C5(C)C)cn2)C4)C3)C1] |
| 220 | 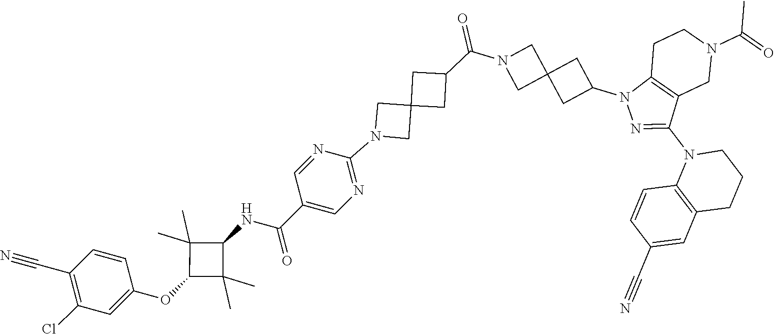 [SMILES: CC(=O)N1CCc2c(c(N3CCCc4cc(C#N)ccc43)nn2C2CC3(C2)CN(C(=O)C2CC4(C2)CN(c2ncc(C(=O)N[C@H]5C(C)(C)[C@H](Oc6ccc(C#N)c(Cl)c6)C5(C)C)cn2)C4)C3)C1] |
| 221 | 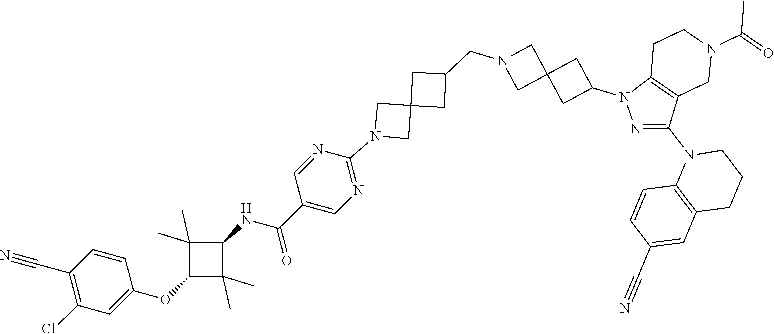 [SMILES: CC(=O)N1CCc2c(c(N3CCCc4cc(C#N)ccc43)nn2C2CC3(C2)CN(CC2CC4(C2)CN(c2ncc(C(=O)N[C@H]5C(C)(C)[C@H](Oc6ccc(C#N)c(Cl)c6)C5(C)C)cn2)C4)C3)C1] |
| 222 | 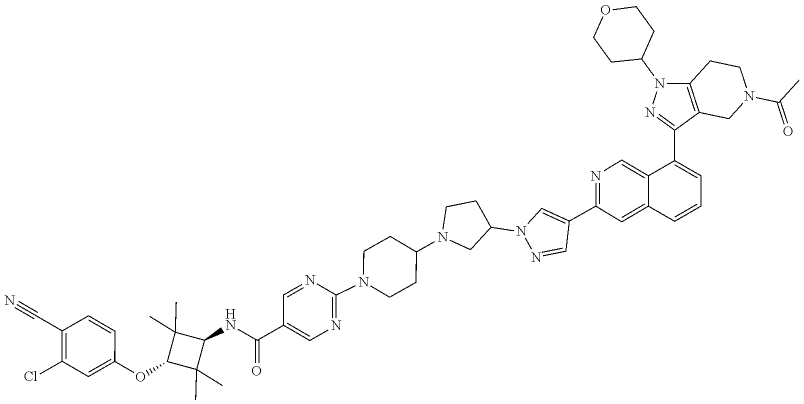 [SMILES: CC(=O)N1CCc2c(c(-c3cccc4cc(-c5cnn(C6CCN(C7CCN(c8ncc(C(=O)N[C@H]9C(C)(C)[C@H](Oc%10ccc(C#N)c(Cl)c%10)C9(C)C)cn8)CC7)C6)c5)ncc34)nn2C2CCOCC2)C1] |
| 223 | 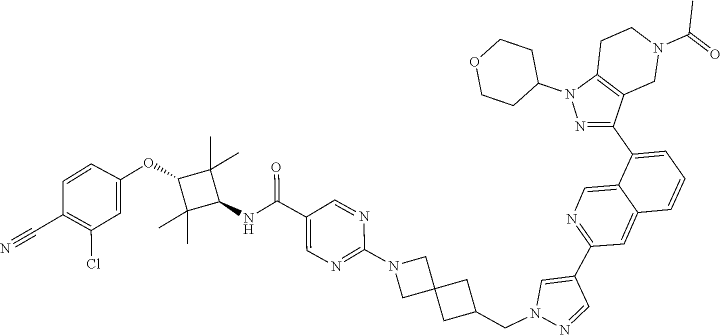 [SMILES: CC(=O)N1CCc2c(c(-c3cccc4cc(-c5cnn(CC6CC7(C6)CN(c6ncc(C(=O)N[C@H]8C(C)(C)[C@H](Oc9ccc(C#N)c(Cl)c9)C8(C)C)cn6)C7)c5)ncc34)nn2C2CCOCC2)C1] |
| 224 | 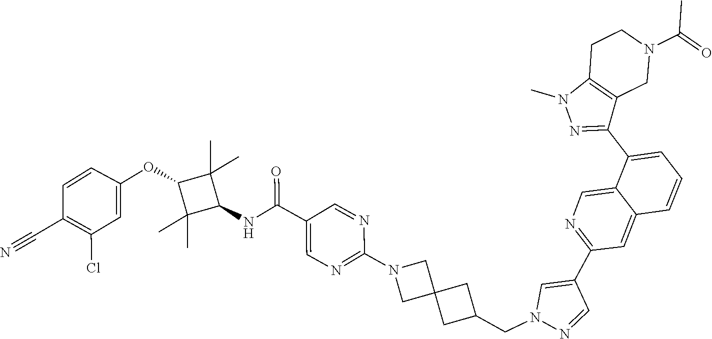 [SMILES: CC(=O)N1CCc2c(c(-c3cccc4cc(-c5cnn(CC6CC7(C6)CN(c6ncc(C(=O)N[C@H]8C(C)(C)[C@H](Oc9ccc(C#N)c(Cl)c9)C8(C)C)cn6)C7)c5)ncc34)nn2C)C1] |
| 225 | 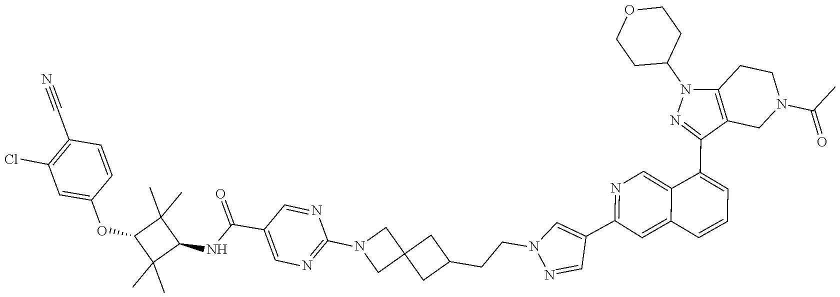 [SMILES: CC(=O)N1CCc2c(c(-c3cccc4cc(-c5cnn(CCC6CC7(C6)CN(c6ncc(C(=O)N[C@H]8C(C)(C)[C@H](Oc9ccc(C#N)c(Cl)c9)C8(C)C)cn6)C7)c5)ncc34)nn2C2CCOCC2)C1] |
| 226 | 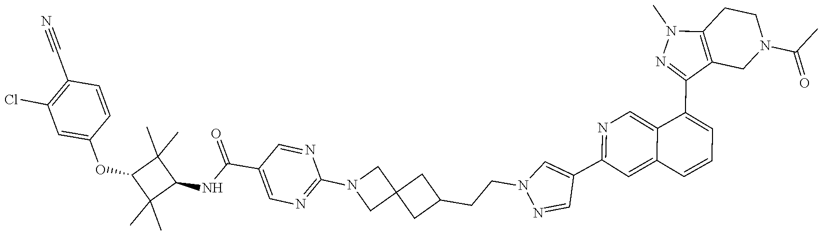 [SMILES: CC(=O)N1CCc2c(c(-c3cccc4cc(-c5cnn(CCC6CC7(C6)CN(c6ncc(C(=O)N[C@H]8C(C)(C)[C@H](Oc9ccc(C#N)c(Cl)c9)C8(C)C)cn6)C7)c5)ncc34)nn2C)C1] |
| 227 | 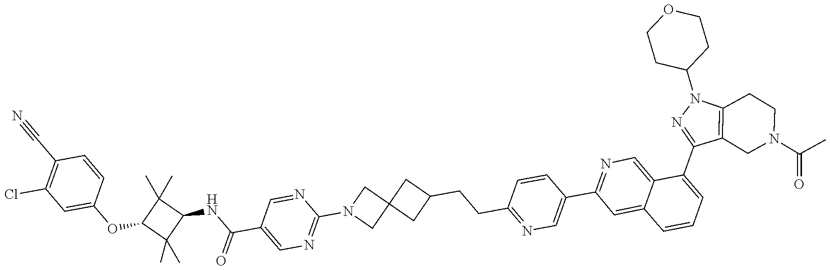 [SMILES: CC(=O)N1CCc2c(c(-c3cccc4cc(-c5ccc(CCC6CC7(C6)CN(c6ncc(C(=O)N[C@H]8C(C)(C)[C@H](Oc9ccc(C#N)c(Cl)c9)C8(C)C)cn6)C7)nc5)ncc34)nn2C2CCOCC2)C1] |
| 228 | 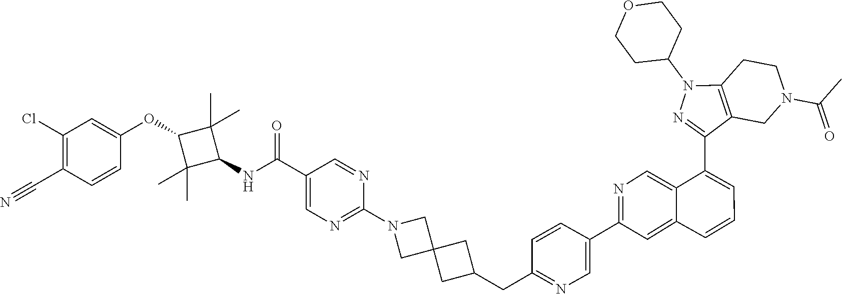 [SMILES: CC(=O)N1CCc2c(c(-c3cccc4cc(-c5ccc(CC6CC7(C6)CN(c6ncc(C(=O)N[C@H]8C(C)(C)[C@H](Oc9ccc(C#N)c(Cl)c9)C8(C)C)cn6)C7)nc5)ncc34)nn2C2CCOCC2)C1] |
| 229 | 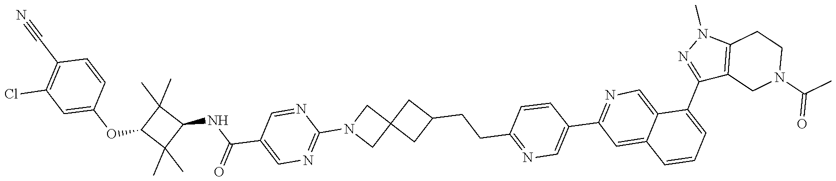 [SMILES: CC(=O)N1CCc2c(c(-c3cccc4cc(-c5ccc(CCC6CC7(C6)CN(c6ncc(C(=O)N[C@H]8C(C)(C)[C@H](Oc9ccc(C#N)c(Cl)c9)C8(C)C)cn6)C7)nc5)ncc34)nn2C)C1] |
| 230 | 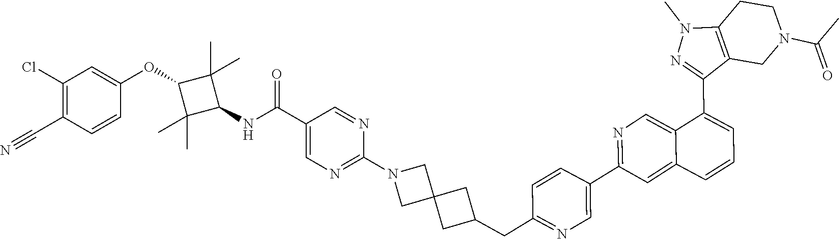 [SMILES: CC(=O)N1CCc2c(c(-c3cccc4cc(-c5ccc(CC6CC7(C6)CN(c6ncc(C(=O)N[C@H]8C(C)(C)[C@H](Oc9ccc(C#N)c(Cl)c9)C8(C)C)cn6)C7)nc5)ncc34)nn2C)C1] |
| 231 | 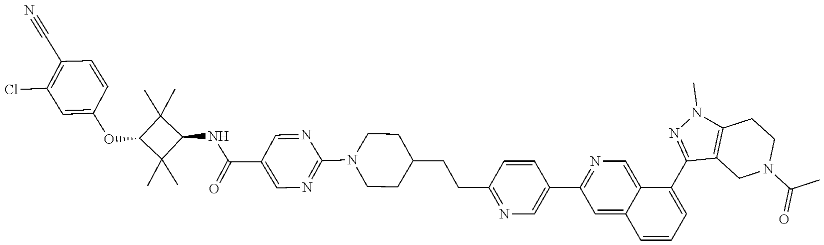 [SMILES: CC(=O)N1CCc2c(c(-c3cccc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc34)nn2C)C1] |
| 232 | 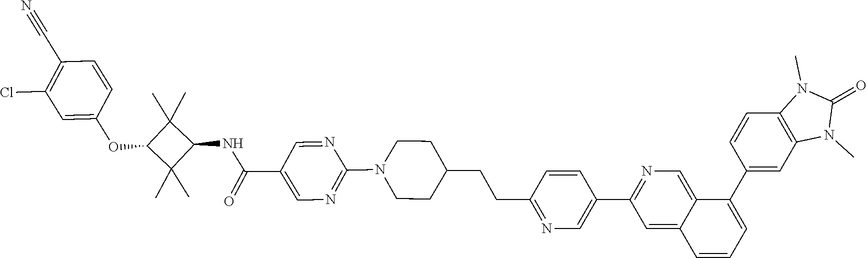 [SMILES: Cn1c(=O)n(C)c2cc(-c3cccc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc34)ccc21] |
| 233 | 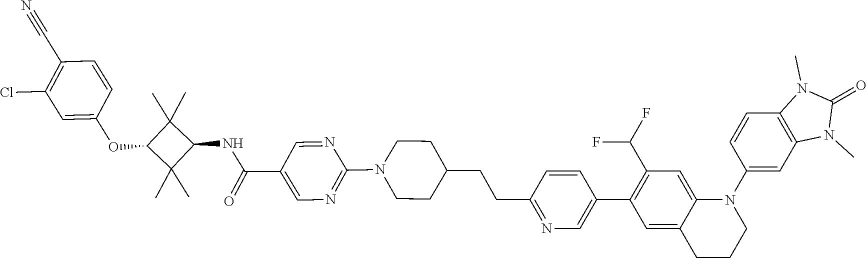 [SMILES: Cn1c(=O)n(C)c2cc(N3CCCc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)c(C(F)F)cc43)ccc21] |
| 234 | 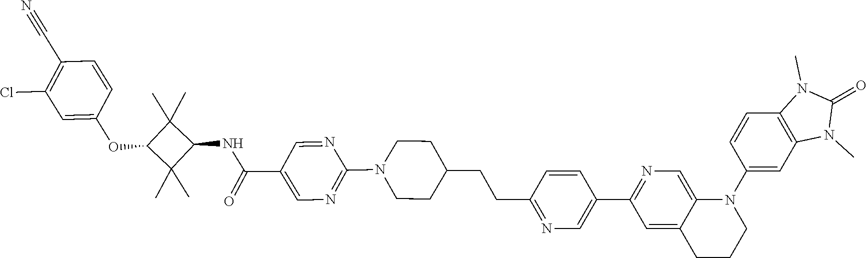 [SMILES: Cn1c(=O)n(C)c2cc(N3CCCc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc43)ccc21] |
| 235 | 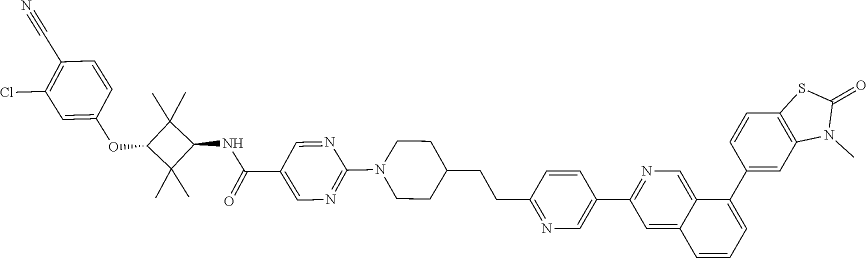 [SMILES: Cn1c(=O)sc2ccc(-c3cccc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc34)cc21] |
| 236 | 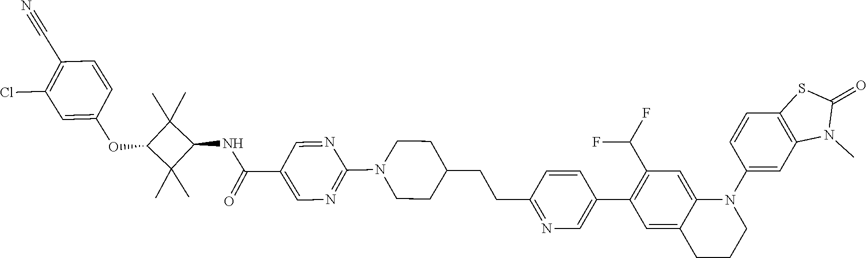 [SMILES: Cn1c(=O)sc2ccc(N3CCCc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)c(C(F)F)cc43)cc21] |
| 237 | 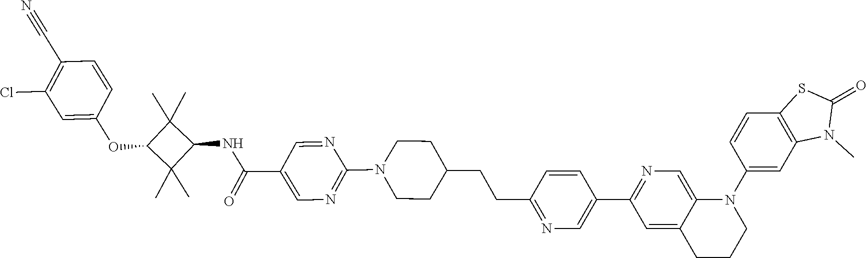 [SMILES: Cn1c(=O)sc2ccc(N3CCCc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc43)cc21] |
| 238 | 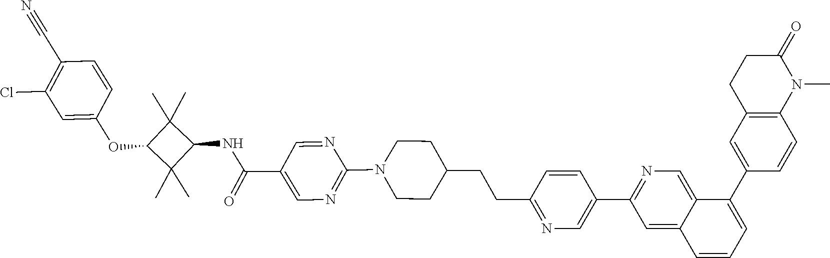 [SMILES: CN1C(=O)CCc2cc(-c3cccc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc34)ccc21] |
| 239 | 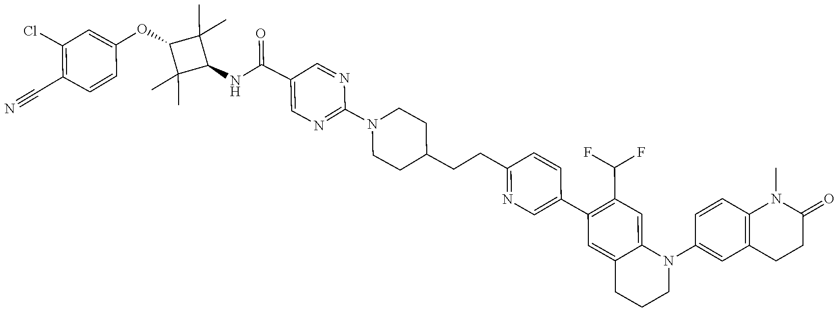 [SMILES: CN1C(=O)CCc2cc(N3CCCc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)c(C(F)F)cc43)ccc21] |
| 240 | 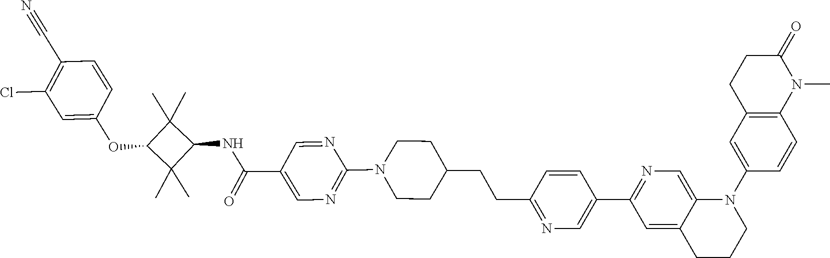 [SMILES: CN1C(=O)CCc2cc(N3CCCc4cc(-c5ccc(CCC6CCN(c7ncc(C(=O)N[C@H]8C(C)(C)[C@H](Oc9ccc(C#N)c(Cl)c9)C8(C)C)cn7)CC6)nc5)ncc43)ccc21] |
| 241 | 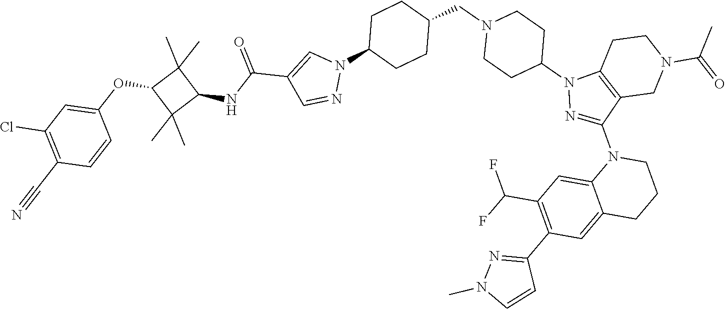 [SMILES: CC(=O)N1CCc2c(c(N3CCCc4cc(-c5ccn(C)n5)c(C(F)F)cc43)nn2C2CCN(C[C@H]3CC[C@H](n4cc(C(=O)N[C@H]5C(C)(C)[C@H](Oc6ccc(C#N)c(Cl)c6)C5(C)C)cn4)CC3)CC2)C1] |
| 242 | 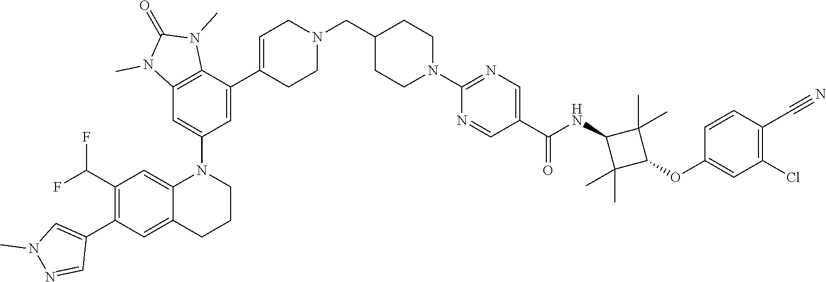 [SMILES: Cn1cc(-c2cc3c(cc2C(F)F)N(c2cc(C4=CCN(CC5CCN(c6ncc(C(=O)N[C@H]7C(C)(C)[C@H](Oc8ccc(C#N)c(Cl)c8)C7(C)C)cn6)CC5)CC4)c4c(c2)n(C)c(=O)n4C)CCC3)cn1] |
| 243 | 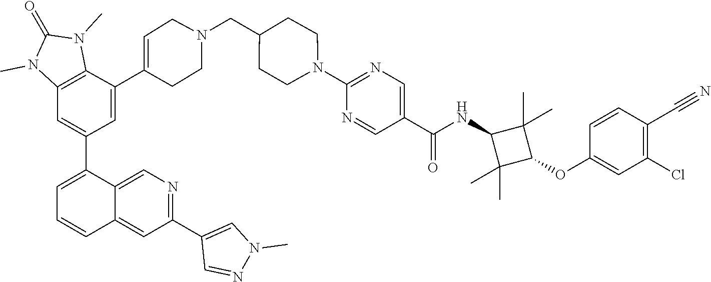 [SMILES: Cn1cc(-c2cc3cccc(-c4cc(C5=CCN(CC6CCN(c7ncc(C(=O)N[C@H]8C(C)(C)[C@H](Oc9ccc(C#N)c(Cl)c9)C8(C)C)cn7)CC6)CC5)c5c(c4)n(C)c(=O)n5C)c3cn2)cn1] |
| 244 | 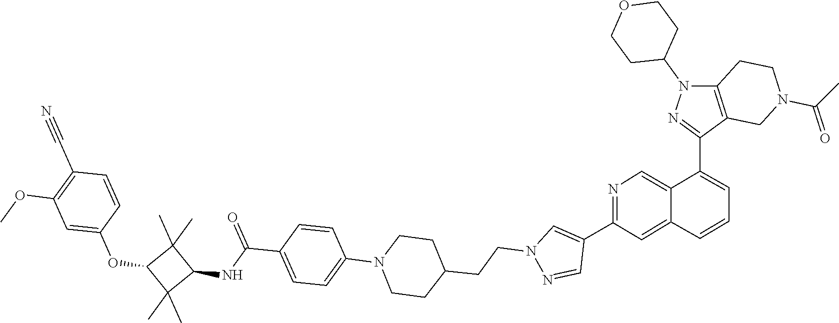 [SMILES: COc1cc(O[C@H]2C(C)(C)[C@H](NC(=O)c3ccc(N4CCC(CCn5cc(-c6cc7cccc(-c8nn(C9CCOCC9)c9c8CN(C(C)=O)CC9)c7cn6)cn5)CC4)cc3)C2(C)C)ccc1C#N] |
| 245 | 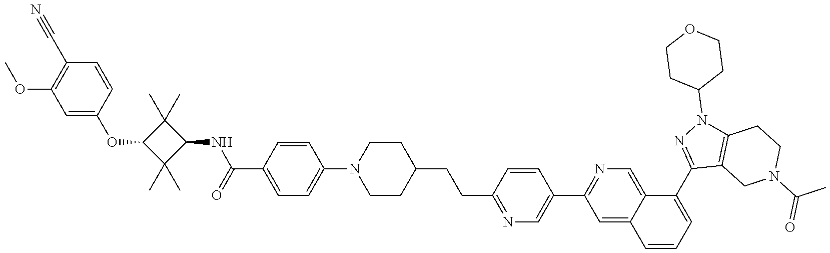 [SMILES: COc1cc(O[C@H]2C(C)(C)[C@H](NC(=O)c3ccc(N4CCC(CCc5ccc(-c6cc7cccc(-c8nn(C9CCOCC9)c9c8CN(C(C)=O)CC9)c7cn6)cn5)CC4)cc3)C2(C)C)ccc1C#N] |
| 246 | 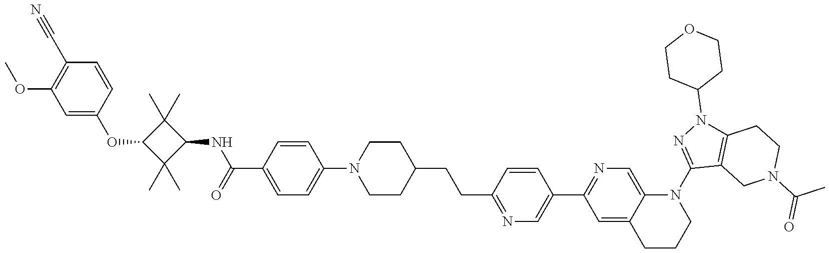 [SMILES: COc1cc(O[C@H]2C(C)(C)[C@H](NC(=O)c3ccc(N4CCC(CCc5ccc(-c6cc7c(cn6)N(c6nn(C8CCOCC8)c8c6CN(C(C)=O)CC8)CCC7)cn5)CC4)cc3)C2(C)C)ccc1C#N] |
| 247 | 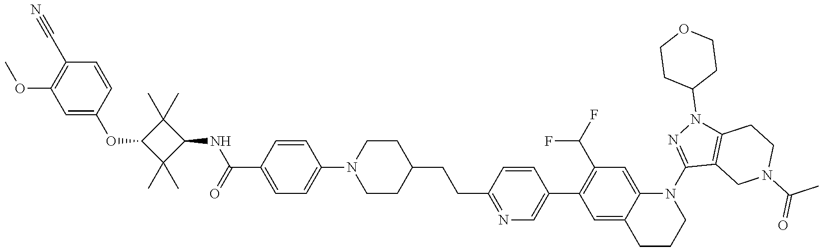 [SMILES: COc1cc(O[C@H]2C(C)(C)[C@H](NC(=O)c3ccc(N4CCC(CCc5ccc(-c6cc7c(cc6C(F)F)N(c6nn(C8CCOCC8)c8c6CN(C(C)=O)CC8)CCC7)cn5)CC4)cc3)C2(C)C)ccc1C#N] |
| 248 | 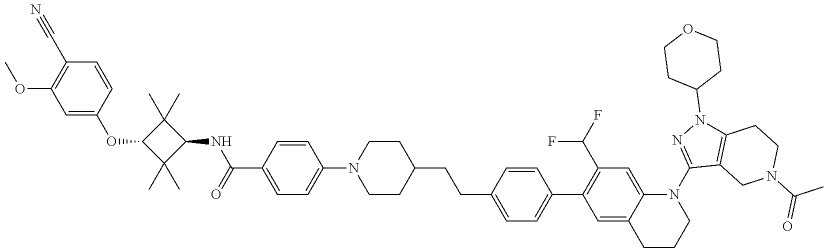 [SMILES: COc1cc(O[C@H]2C(C)(C)[C@H](NC(=O)c3ccc(N4CCC(CCc5ccc(-c6cc7c(cc6C(F)F)N(c6nn(C8CCOCC8)c8c6CN(C(C)=O)CC8)CCC7)cc5)CC4)cc3)C2(C)C)ccc1C#N] |
| 249 | 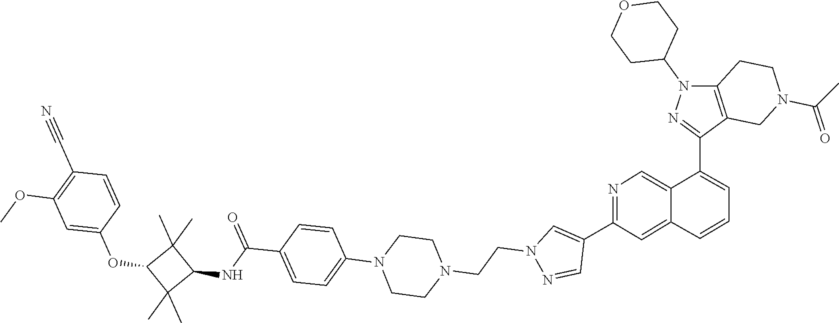 [SMILES: COc1cc(O[C@H]2C(C)(C)[C@H](NC(=O)c3ccc(N4CCN(CCn5cc(-c6cc7cccc(-c8nn(C9CCOCC9)c9c8CN(C(C)=O)CC9)c7cn6)cn5)CC4)cc3)C2(C)C)ccc1C#N] |
| 250 | 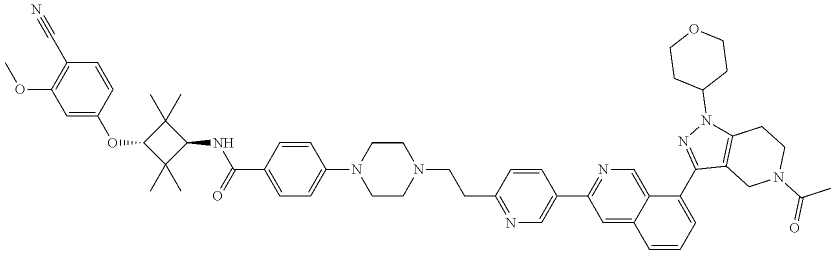 [SMILES: COc1cc(O[C@H]2C(C)(C)[C@H](NC(=O)c3ccc(N4CCN(CCc5ccc(-c6cc7cccc(-c8nn(C9CCOCC9)c9c8CN(C(C)=O)CC9)c7cn6)cn5)CC4)cc3)C2(C)C)ccc1C#N] |
| 251 | 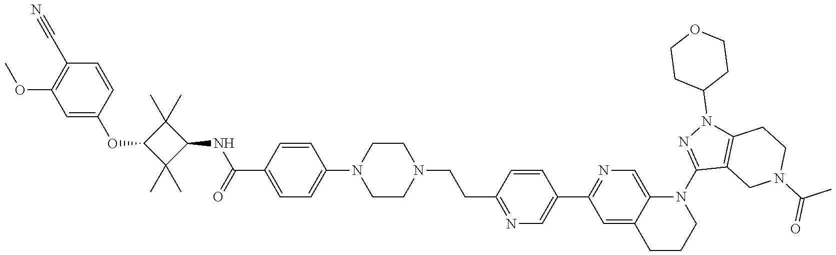 [SMILES: COc1cc(O[C@H]2C(C)(C)[C@H](NC(=O)c3ccc(N4CCN(CCc5ccc(-c6cc7c(cn6)N(c6nn(C8CCOCC8)c8c6CN(C(C)=O)CC8)CCC7)cn5)CC4)cc3)C2(C)C)ccc1C#N] |
| 252 | 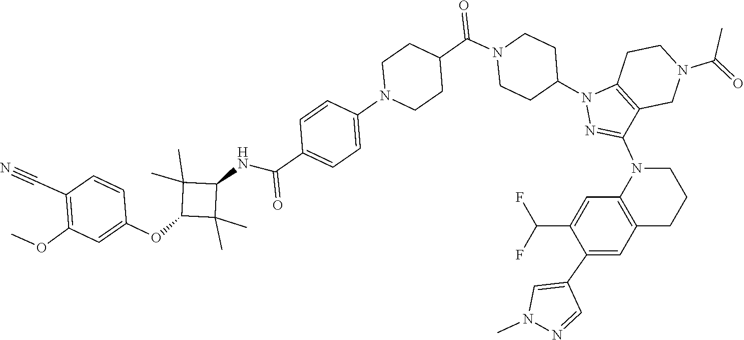 [SMILES: COc1cc(O[C@H]2C(C)(C)[C@H](NC(=O)c3ccc(N4CCC(C(=O)N5CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 253 | 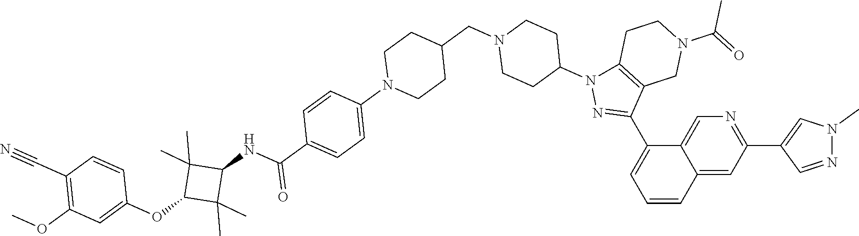 [SMILES: COc1cc(O[C@H]2C(C)(C)[C@H](NC(=O)c3ccc(N4CCC(CN5CCC(n6nc(-c7cccc8cc(-c9cnn(C)c9)ncc78)c7c6CCN(C(C)=O)C7)CC5)CC4)cc3)C2(C)C)ccc1C#N] |
| 254 | 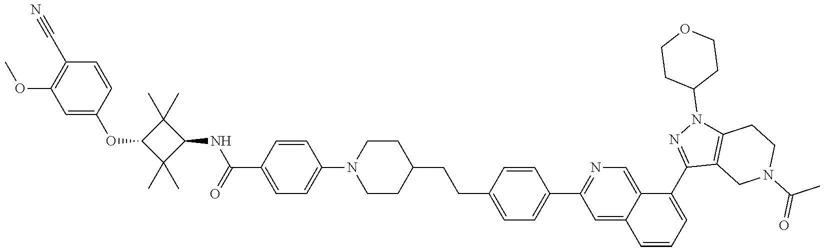 [SMILES: COc1cc(O[C@H]2C(C)(C)[C@H](NC(=O)c3ccc(N4CCC(CCc5ccc(-c6cc7cccc(-c8nn(C9CCOCC9)c9c8CN(C(C)=O)CC9)c7cn6)cc5)CC4)cc3)C2(C)C)ccc1C#N] |
| 255 | 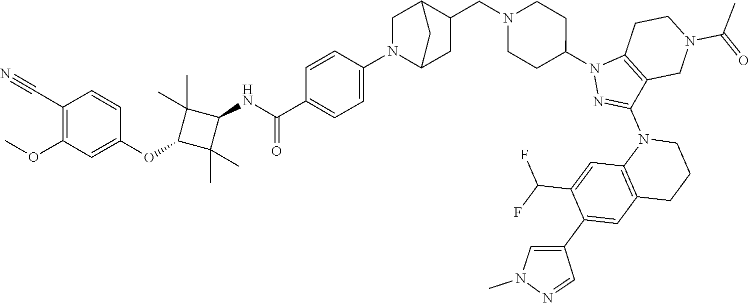 [SMILES: COc1cc(O[C@H]2C(C)(C)[C@H](NC(=O)c3ccc(N4CC5CC4CC5CN4CCC(n5nc(N6CCCc7cc(-c8cnn(C)c8)c(C(F)F)cc76)c6c5CCN(C(C)=O)C6)CC4)cc3)C2(C)C)ccc1C#N] |
| 256 | 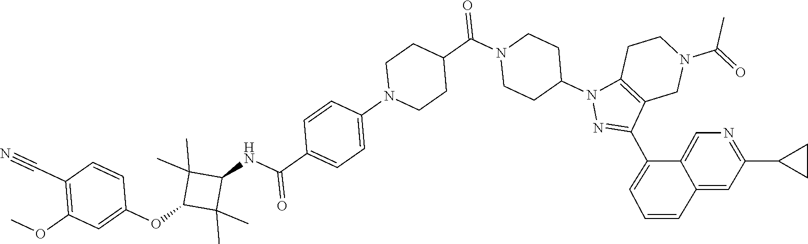 [SMILES: COc1cc(O[C@H]2C(C)(C)[C@H](NC(=O)c3ccc(N4CCC(C(=O)N5CCC(n6nc(-c7cccc8cc(C9CC9)ncc78)c7c6CCN(C(C)=O)C7)CC5)CC4)cc3)C2(C)C)ccc1C#N] |
| 257 | 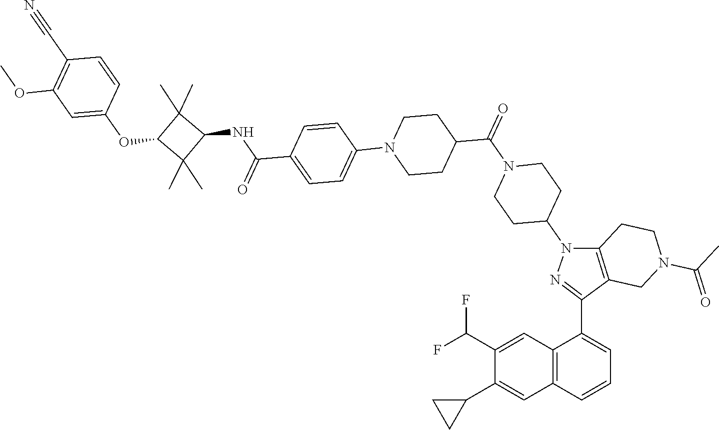 [SMILES: COc1cc(O[C@H]2C(C)(C)[C@H](NC(=O)c3ccc(N4CCC(C(=O)N5CCC(n6nc(-c7cccc8cc(C9CC9)c(C(F)F)cc78)c7c6CCN(C(C)=O)C7)CC5)CC4)cc3)C2(C)C)ccc1C#N] |
| 258 | 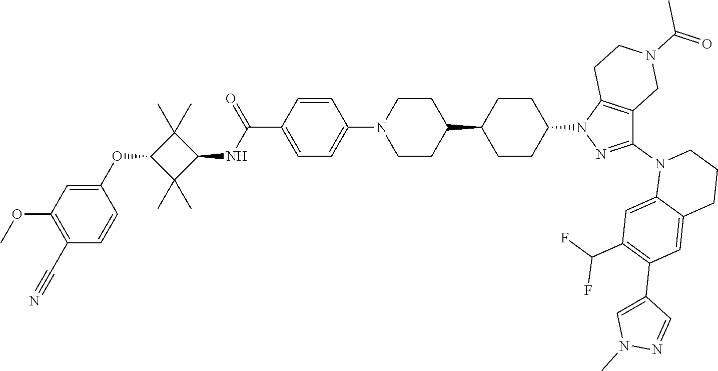 [SMILES: COc1cc(O[C@H]2C(C)(C)[C@H](NC(=O)c3ccc(N4CCC([C@H]5CC[C@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 259 | 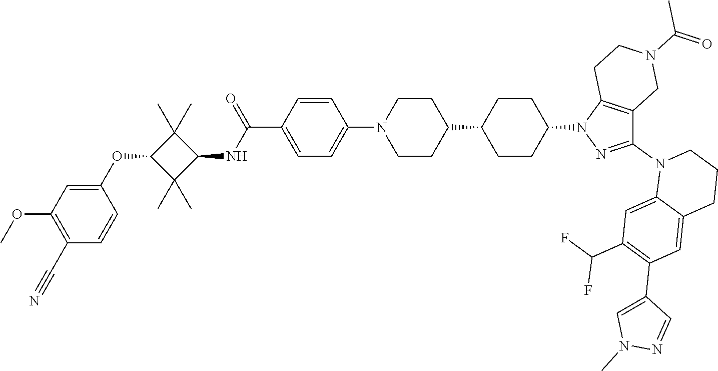 [SMILES: COc1cc(O[C@H]2C(C)(C)[C@H](NC(=O)c3ccc(N4CCC([C@H]5CC[C@@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 260 | 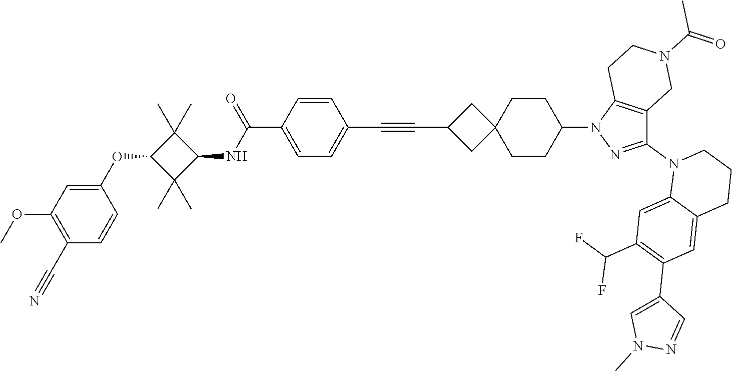 [SMILES: COc1cc(O[C@H]2C(C)(C)[C@H](NC(=O)c3ccc(C#CC4CC5(CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 261 | 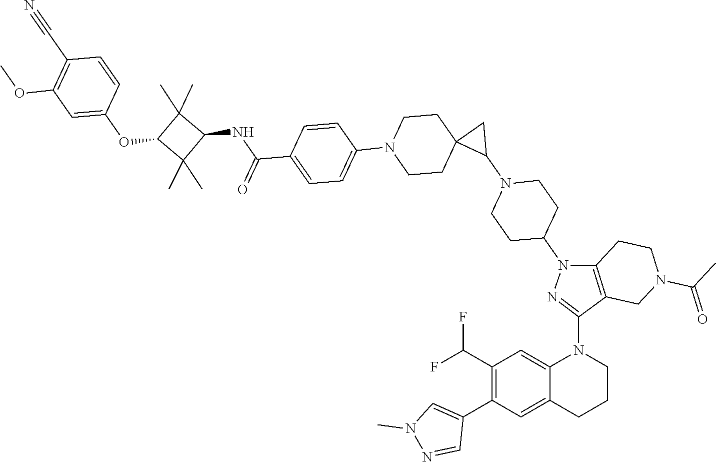 [SMILES: COc1cc(O[C@H]2C(C)(C)[C@H](NC(=O)c3ccc(N4CCC5(CC4)CC5N4CCC(n5nc(N6CCCc7cc(-c8cnn(C)c8)c(C(F)F)cc76)c6c5CCN(C(C)=O)C6)CC4)cc3)C2(C)C)ccc1C#N] |
| 262 | 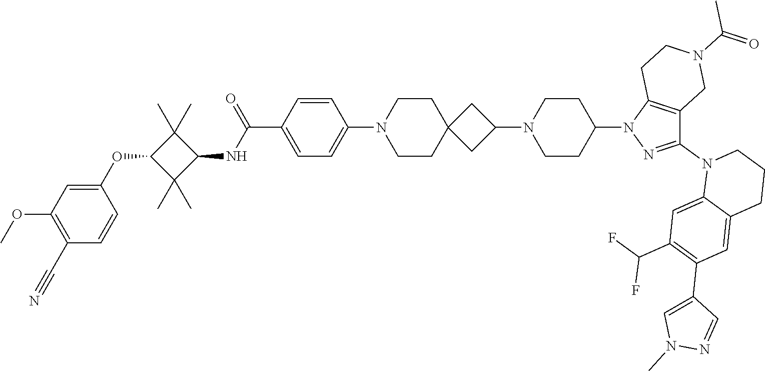 [SMILES: COc1cc(O[C@H]2C(C)(C)[C@H](NC(=O)c3ccc(N4CCC5(CC4)CC(N4CCC(n6nc(N7CCCc8cc(-c9cnn(C)c9)c(C(F)F)cc87)c7c6CCN(C(C)=O)C7)CC4)C5)cc3)C2(C)C)ccc1C#N] |
| 263 | 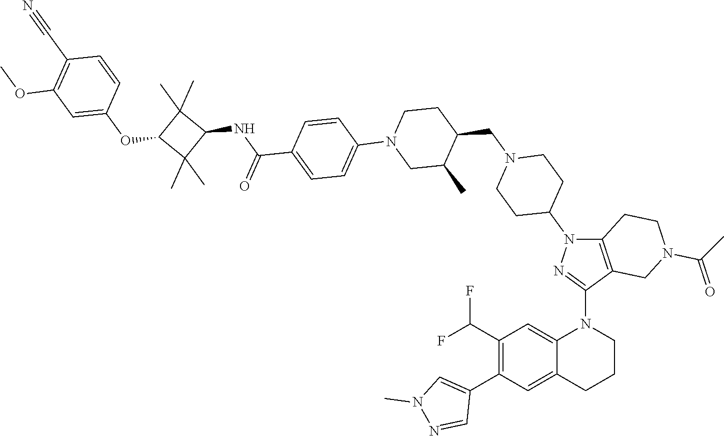 [SMILES: COc1cc(O[C@H]2C(C)(C)[C@H](NC(=O)c3ccc(N4CC[C@@H](CN5CCC(n6nc(N7CCCc8cc(-c9cnn(C)c9)c(C(F)F)cc87)c7c6CCN(C(C)=O)C7)CC5)[C@@H](C)C4)cc3)C2(C)C)ccc1C#N] |
| 264 | 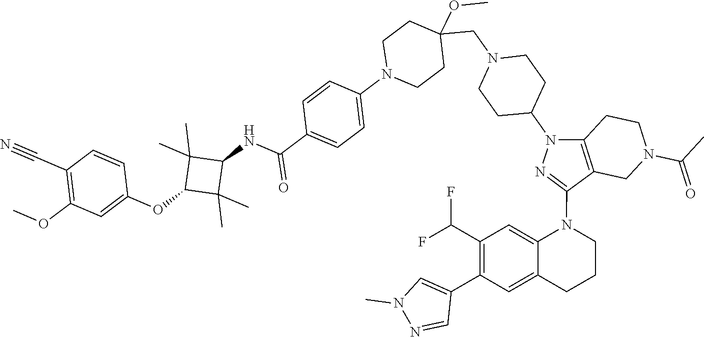 [SMILES: COc1cc(O[C@H]2C(C)(C)[C@H](NC(=O)c3ccc(N4CCC(CN5CCC(n6nc(N7CCCc8cc(-c9cnn(C)c9)c(C(F)F)cc87)c7c6CCN(C(C)=O)C7)CC5)(OC)CC4)cc3)C2(C)C)ccc1C#N] |
| 265 | 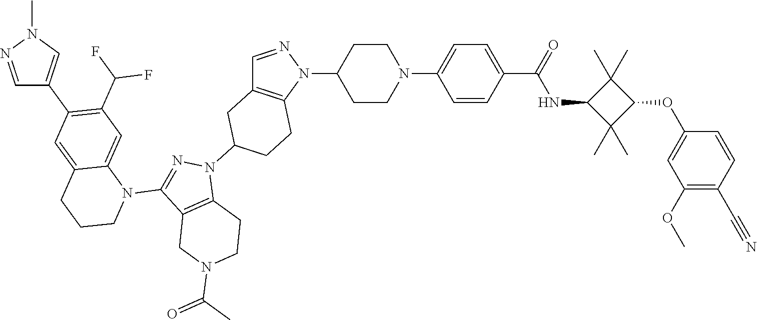 [SMILES: COc1cc(O[C@H]2C(C)(C)[C@H](NC(=O)c3ccc(N4CCC(n5ncc6c5CCC(n5nc(N7CCCc8cc(-c9cnn(C)c9)c(C(F)F)cc87)c7c5CCN(C(C)=O)C7)C6)CC4)cc3)C2(C)C)ccc1C#N] |
| 266 | 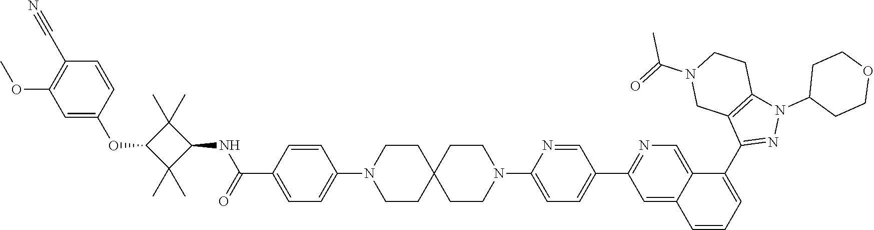 [SMILES: COc1cc(O[C@H]2C(C)(C)[C@H](NC(=O)c3ccc(N4CCC5(CC4)CCN(c4ccc(-c6cc7cccc(-c8nn(C9CCOCC9)c9c8CN(C(C)=O)CC9)c7cn6)cn4)CC5)cc3)C2(C)C)ccc1C#N] |
| 267 | 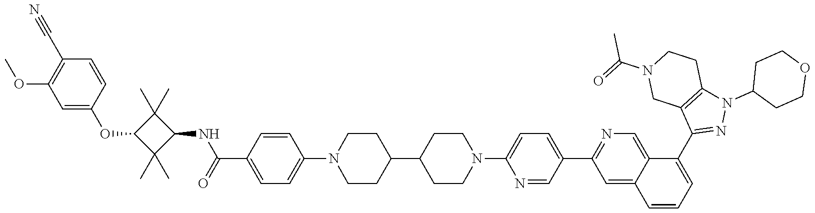 [SMILES: COc1cc(O[C@H]2C(C)(C)[C@H](NC(=O)c3ccc(N4CCC(C5CCN(c6ccc(-c7cc8cccc(-c9nn(C%10CCOCC%10)c%10c9CN(C(C)=O)CC%10)c8cn7)cn6)CC5)CC4)cc3)C2(C)C)ccc1C#N] |
| 268 | 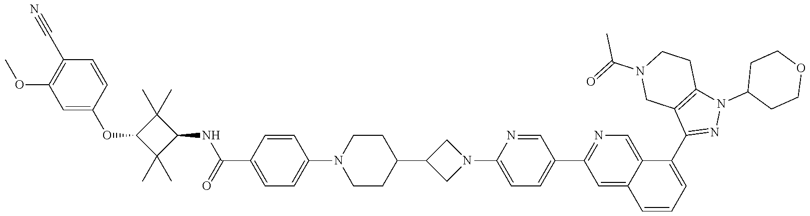 [SMILES: COc1cc(O[C@H]2C(C)(C)[C@H](NC(=O)c3ccc(N4CCC(C5CN(c6ccc(-c7cc8cccc(-c9nn(C%10CCOCC%10)c%10c9CN(C(C)=O)CC%10)c8cn7)cn6)C5)CC4)cc3)C2(C)C)ccc1C#N] |
| 269 | 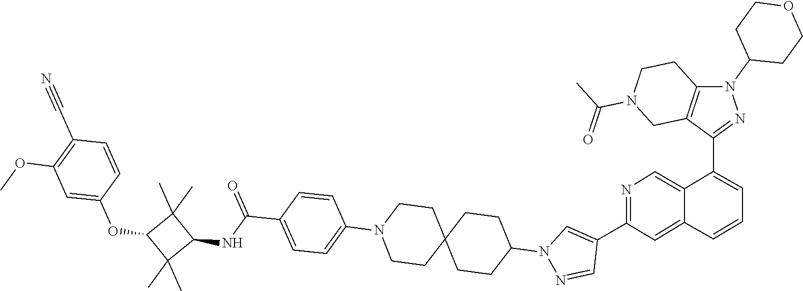 [SMILES: COc1cc(O[C@H]2C(C)(C)[C@H](NC(=O)c3ccc(N4CCC5(CCC(n6cc(-c7cc8cccc(-c9nn(C%10CCOCC%10)c%10c9CN(C(C)=O)CC%10)c8cn7)cn6)CC5)CC4)cc3)C2(C)C)ccc1C#N] |
| 270 | 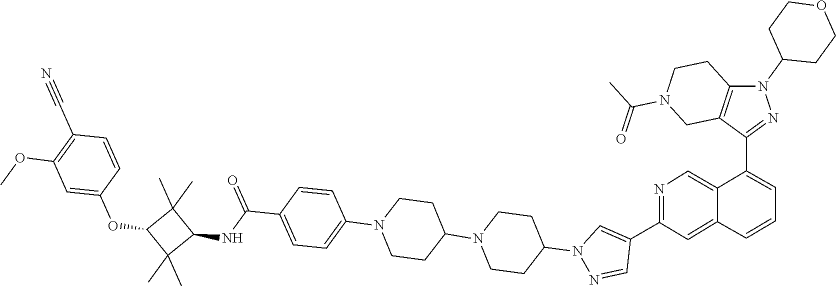 [SMILES: COc1cc(O[C@H]2C(C)(C)[C@H](NC(=O)c3ccc(N4CCC(N5CCC(n6cc(-c7cc8cccc(-c9nn(C%10CCOCC%10)c%10c9CN(C(C)=O)CC%10)c8cn7)cn6)CC5)CC4)cc3)C2(C)C)ccc1C#N] |
| 271 | 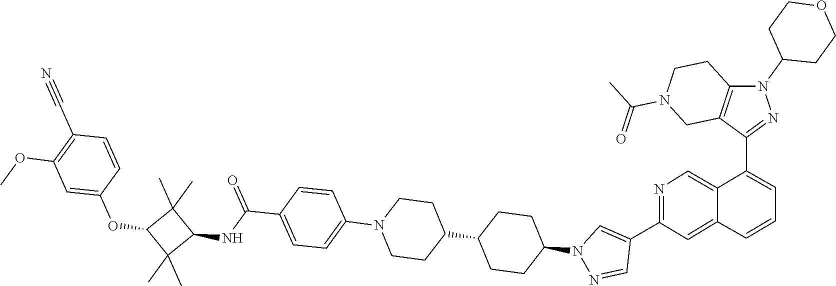 [SMILES: COc1cc(O[C@H]2C(C)(C)[C@H](NC(=O)c3ccc(N4CCC([C@H]5CC[C@H](n6cc(-c7cc8cccc(-c9nn(C%10CCOCC%10)c%10c9CN(C(C)=O)CC%10)c8cn7)cn6)CC5)CC4)cc3)C2(C)C)ccc1C#N] |
| 272 | 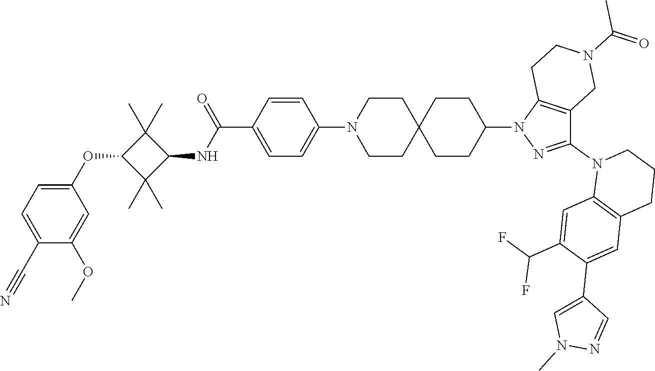 [SMILES: COc1cc(O[C@H]2C(C)(C)[C@H](NC(=O)c3ccc(N4CCC5(CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 273 | 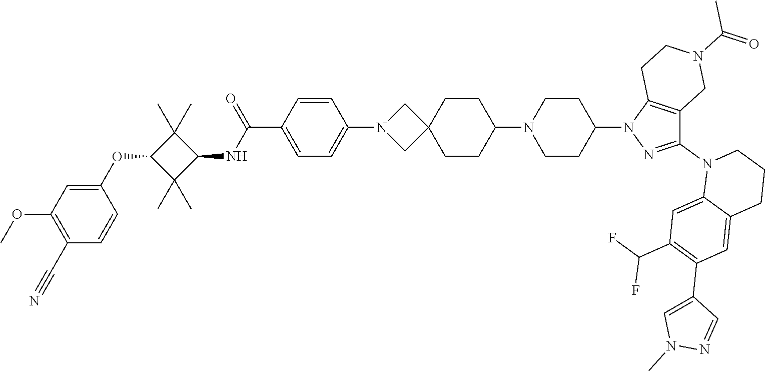 [SMILES: COc1cc(O[C@H]2C(C)(C)[C@H](NC(=O)c3ccc(N4CC5(CCC(N6CCC(n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 274 | 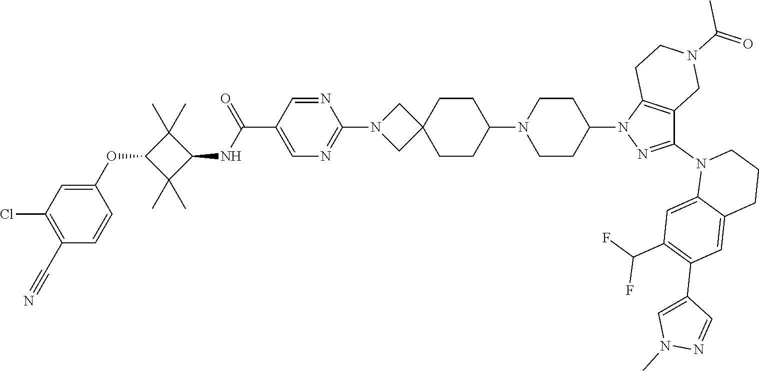 [SMILES: CC(=O)N1CCc2c(c(N3CCCc4cc(-c5cnn(C)c5)c(C(F)F)cc43)nn2C2CCN(C3CCC4(CC3)CN(c3ncc(C(=O)N[C@H]5C(C)(C)[C@H](Oc6ccc(C#N)c(Cl)c6)C5(C)C)cn3)C4)CC2)C1] |
| 275 | 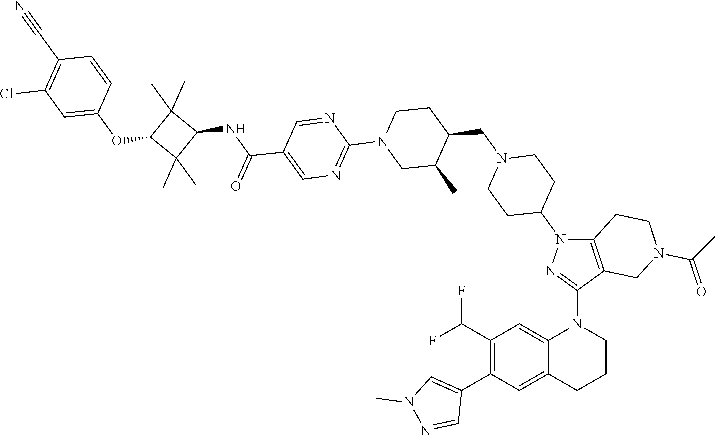 [SMILES: CC(=O)N1CCc2c(c(N3CCCc4cc(-c5cnn(C)c5)c(C(F)F)cc43)nn2C2CCN(C[C@@H]3CCN(c4ncc(C(=O)N[C@H]5C(C)(C)[C@H](Oc6ccc(C#N)c(Cl)c6)C5(C)C)cn4)C[C@@H]3C)CC2)C1] |
| 276 | 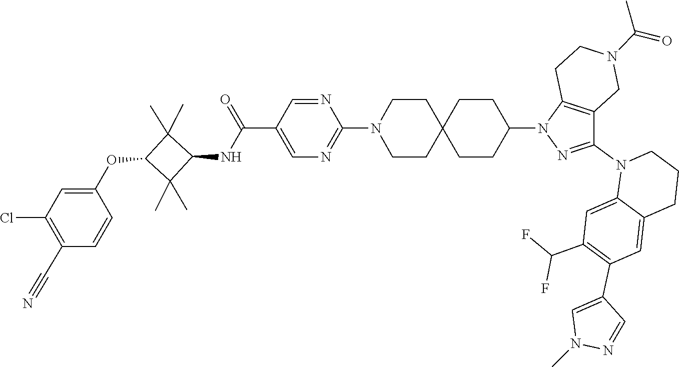 [SMILES: CC(=O)N1CCc2c(c(N3CCCc4cc(-c5cnn(C)c5)c(C(F)F)cc43)nn2C2CCC3(CC2)CCN(c2ncc(C(=O)N[C@H]4C(C)(C)[C@H](Oc5ccc(C#N)c(Cl)c5)C4(C)C)cn2)CC3)C1] |
| 277 | 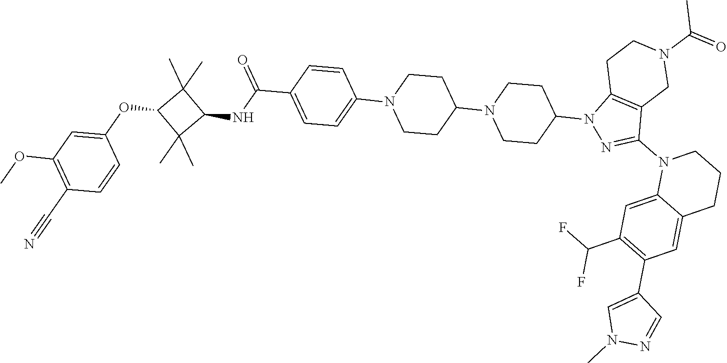 [SMILES: COc1cc(O[C@H]2C(C)(C)[C@H](NC(=O)c3ccc(N4CCC(N5CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 278 | 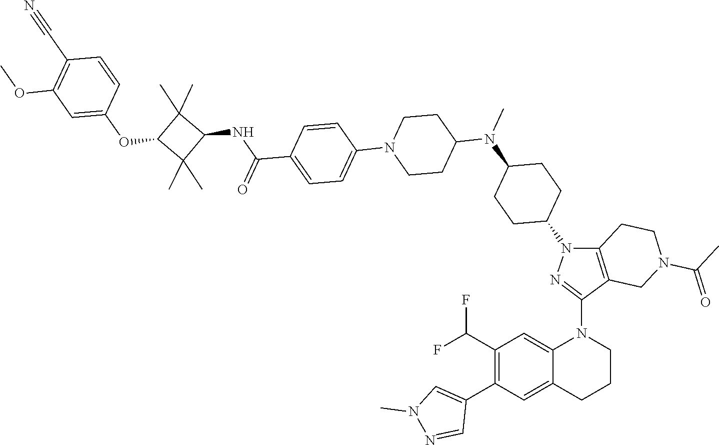 [SMILES: COc1cc(O[C@H]2C(C)(C)[C@H](NC(=O)c3ccc(N4CCC(N(C)[C@H]5CC[C@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 279 | 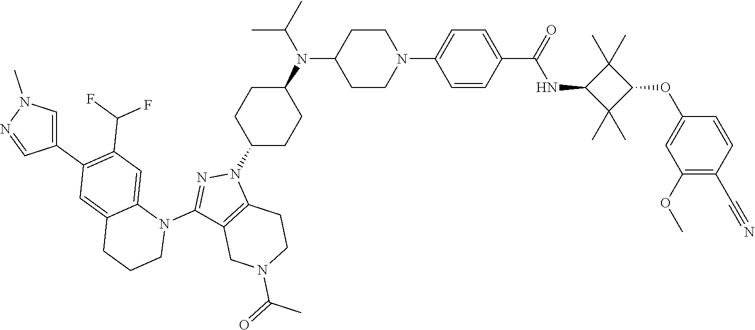 [SMILES: COc1cc(O[C@H]2C(C)(C)[C@H](NC(=O)c3ccc(N4CCC(N(C(C)C)[C@H]5CC[C@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 280 | 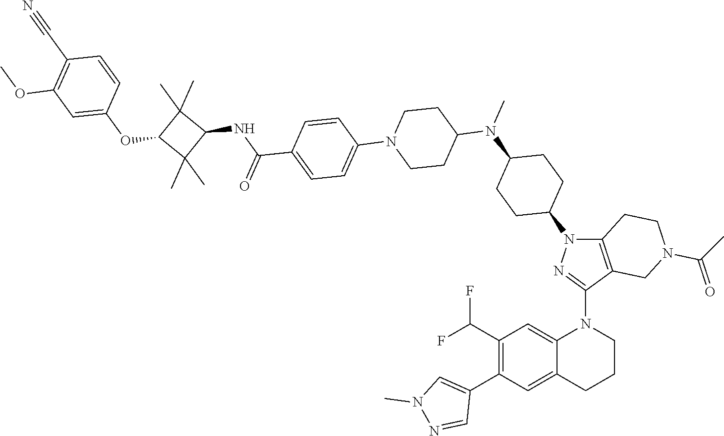 [SMILES: COc1cc(O[C@H]2C(C)(C)[C@H](NC(=O)c3ccc(N4CCC(N(C)[C@H]5CC[C@@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 281 | 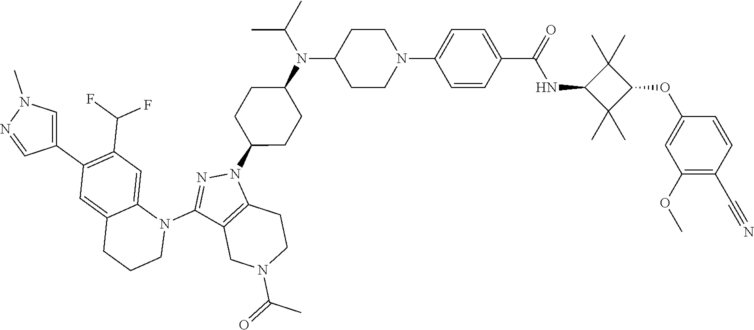 [SMILES: COc1cc(O[C@H]2C(C)(C)[C@H](NC(=O)c3ccc(N4CCC(N(C(C)C)[C@H]5CC[C@@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 282 | 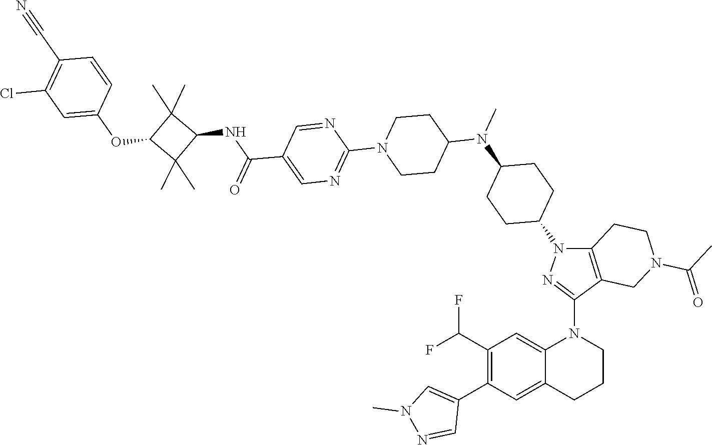 [SMILES: CC(=O)N1CCc2c(c(N3CCCc4cc(-c5cnn(C)c5)c(C(F)F)cc43)nn2[C@H]2CC[C@H](N(C)C3CCN(c4ncc(C(=O)N[C@H]5C(C)(C)[C@H](Oc6ccc(C#N)c(Cl)c6)C5(C)C)cn4)CC3)CC2)C1] |
| 283 | 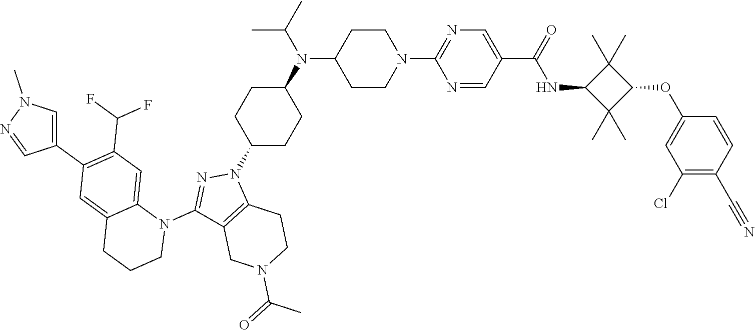 [SMILES: CC(=O)N1CCc2c(c(N3CCCc4cc(-c5cnn(C)c5)c(C(F)F)cc43)nn2[C@H]2CC[C@H](N(C(C)C)C3CCN(c4ncc(C(=O)N[C@H]5C(C)(C)[C@H](Oc6ccc(C#N)c(Cl)c6)C5(C)C)cn4)CC3)CC2)C1] |
| 284 | 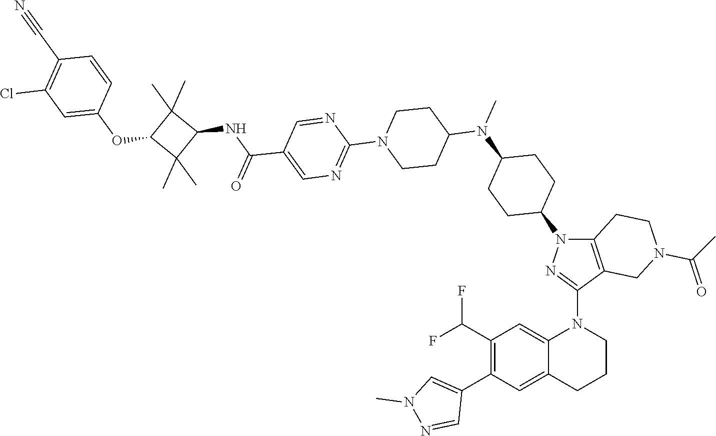 [SMILES: CC(=O)N1CCc2c(c(N3CCCc4cc(-c5cnn(C)c5)c(C(F)F)cc43)nn2[C@H]2CC[C@@H](N(C)C3CCN(c4ncc(C(=O)N[C@H]5C(C)(C)[C@H](Oc6ccc(C#N)c(Cl)c6)C5(C)C)cn4)CC3)CC2)C1] |
| 285 | 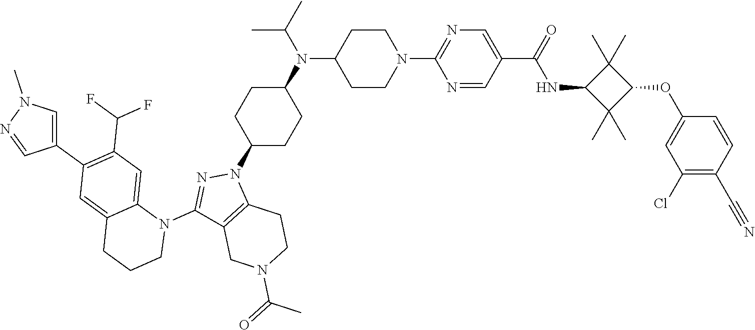 [SMILES: CC(=O)N1CCc2c(c(N3CCCc4cc(-c5cnn(C)c5)c(C(F)F)cc43)nn2[C@H]2CC[C@@H](N(C(C)C)C3CCN(c4ncc(C(=O)N[C@H]5C(C)(C)[C@H](Oc6ccc(C#N)c(Cl)c6)C5(C)C)cn4)CC3)CC2)C1] |
| 286 | 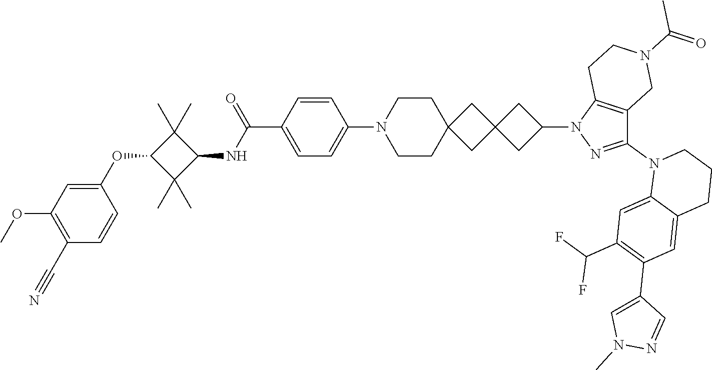 [SMILES: COc1cc(O[C@H]2C(C)(C)[C@H](NC(=O)c3ccc(N4CCC5(CC4)CC4(CC(n6nc(N7CCCc8cc(-c9cnn(C)c9)c(C(F)F)cc87)c7c6CCN(C(C)=O)C7)C4)C5)cc3)C2(C)C)ccc1C#N] |
| 287 | 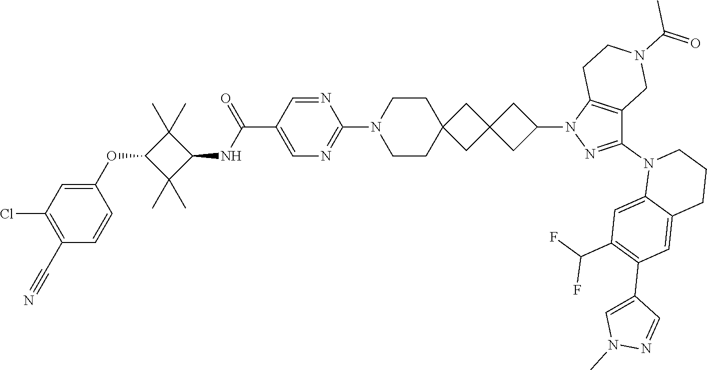 [SMILES: CC(=O)N1CCc2c(c(N3CCCc4cc(-c5cnn(C)c5)c(C(F)F)cc43)nn2C2CC3(C2)CC2(CCN(c4ncc(C(=O)N[C@H]5C(C)(C)[C@H](Oc6ccc(C#N)c(Cl)c6)C5(C)C)cn4)CC2)C3)C1] |
| 288 | 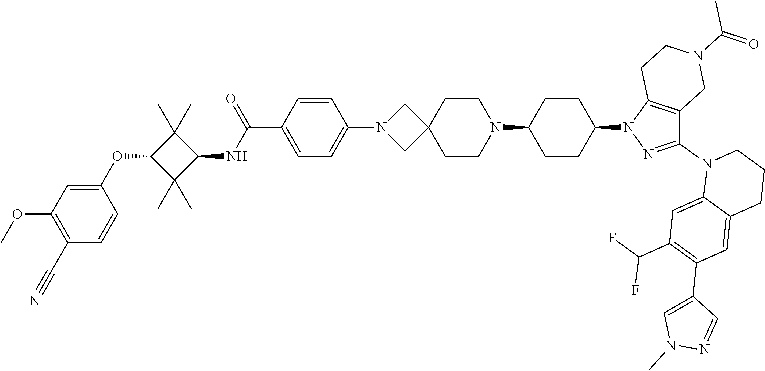 [SMILES: COc1cc(O[C@H]2C(C)(C)[C@H](NC(=O)c3ccc(N4CC5(CCN([C@H]6CC[C@@H](n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 289 | 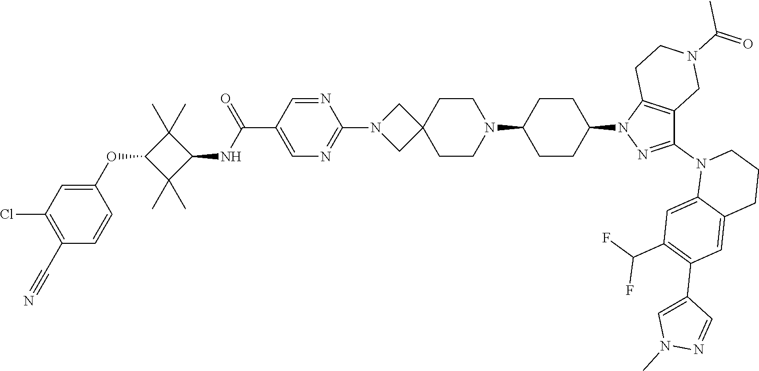 [SMILES: CC(=O)N1CCc2c(c(N3CCCc4cc(-c5cnn(C)c5)c(C(F)F)cc43)nn2[C@H]2CC[C@@H](N3CCC4(CC3)CN(c3ncc(C(=O)N[C@H]5C(C)(C)[C@H](Oc6ccc(C#N)c(Cl)c6)C5(C)C)cn3)C4)CC2)C1] |
| 290 | 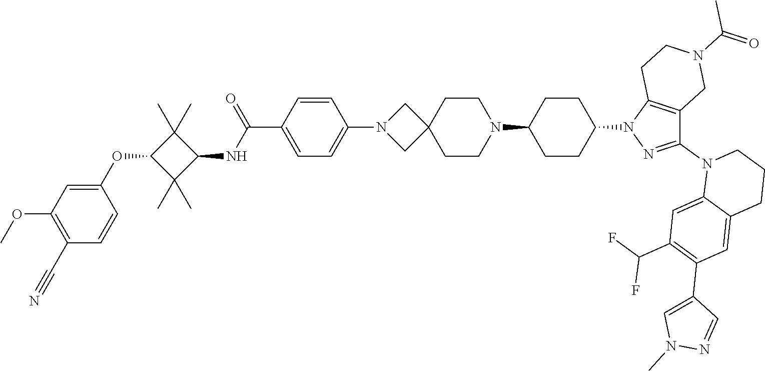 [SMILES: COc1cc(O[C@H]2C(C)(C)[C@H](NC(=O)c3ccc(N4CC5(CCN([C@H]6CC[C@H](n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 291 | 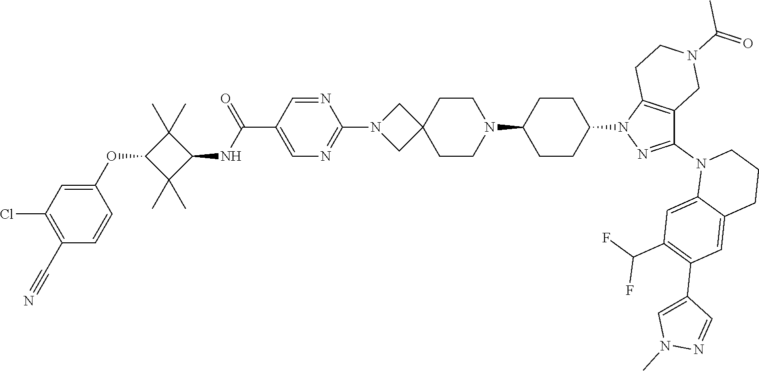 [SMILES: CC(=O)N1CCc2c(c(N3CCCc4cc(-c5cnn(C)c5)c(C(F)F)cc43)nn2[C@H]2CC[C@H](N3CCC4(CC3)CN(c3ncc(C(=O)N[C@H]5C(C)(C)[C@H](Oc6ccc(C#N)c(Cl)c6)C5(C)C)cn3)C4)CC2)C1] |
| 292 | 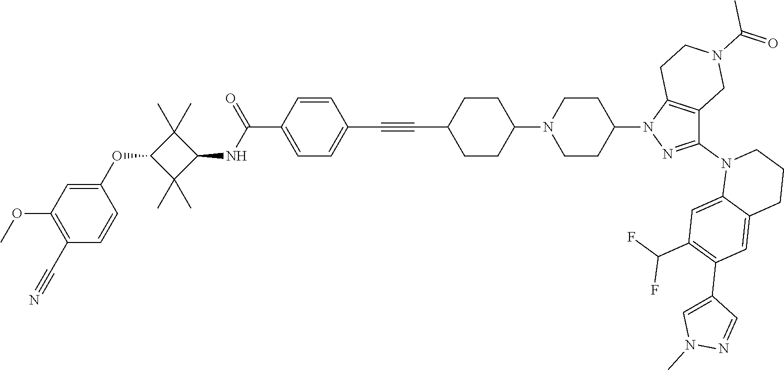 [SMILES: COc1cc(O[C@H]2C(C)(C)[C@H](NC(=O)c3ccc(C#CC4CCC(N5CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 293 | 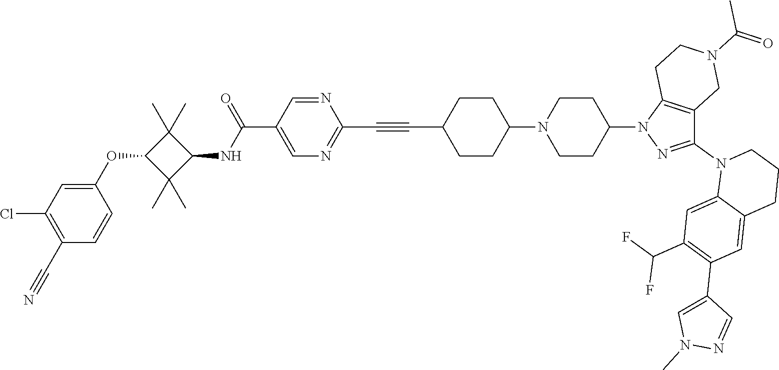 [SMILES: CC(=O)N1CCc2c(c(N3CCCc4cc(-c5cnn(C)c5)c(C(F)F)cc43)nn2C2CCN(C3CCC(C#Cc4ncc(C(=O)N[C@H]5C(C)(C)[C@H](Oc6ccc(C#N)c(Cl)c6)C5(C)C)cn4)CC3)CC2)C1] |
| 294 | 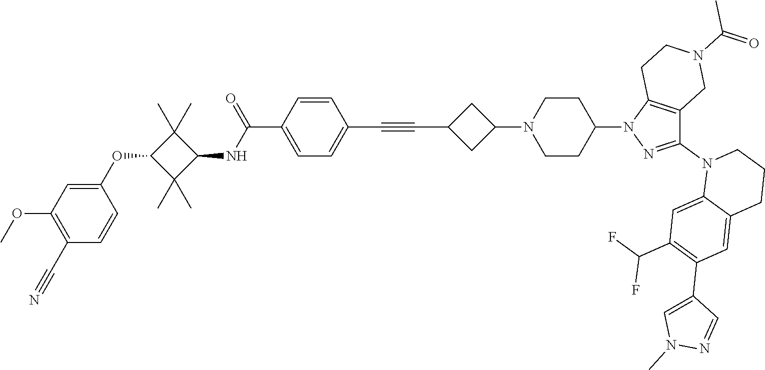 [SMILES: COc1cc(O[C@H]2C(C)(C)[C@H](NC(=O)c3ccc(C#CC4CC(N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 295 | 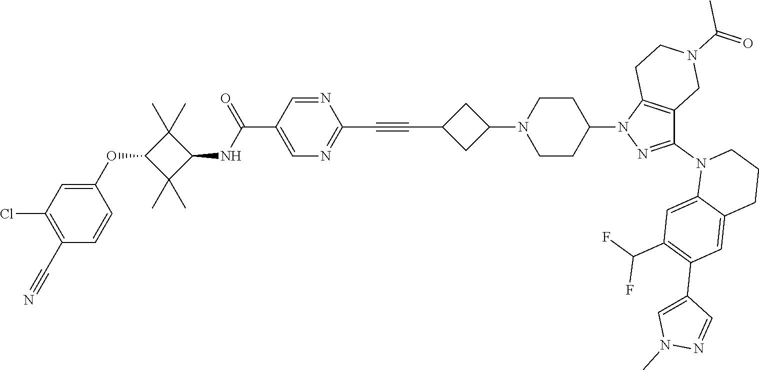 [SMILES: CC(=O)N1CCc2c(c(N3CCCc4cc(-c5cnn(C)c5)c(C(F)F)cc43)nn2C2CCN(C3CC(C#Cc4ncc(C(=O)N[C@H]5C(C)(C)[C@H](Oc6ccc(C#N)c(Cl)c6)C5(C)C)cn4)C3)CC2)C1] |
| 296 | 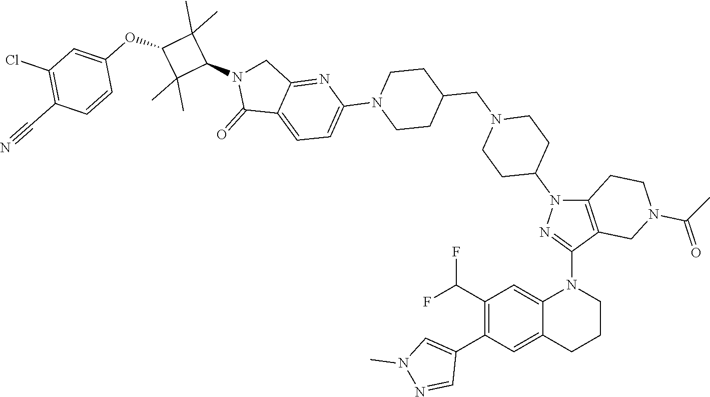 [SMILES: CC(=O)N1CCc2c(c(N3CCCc4cc(-c5cnn(C)c5)c(C(F)F)cc43)nn2C2CCN(CC3CCN(c4ccc5c(n4)CN([C@H]4C(C)(C)[C@H](Oc6ccc(C#N)c(Cl)c6)C4(C)C)C5=O)CC3)CC2)C1] |
| 297 | 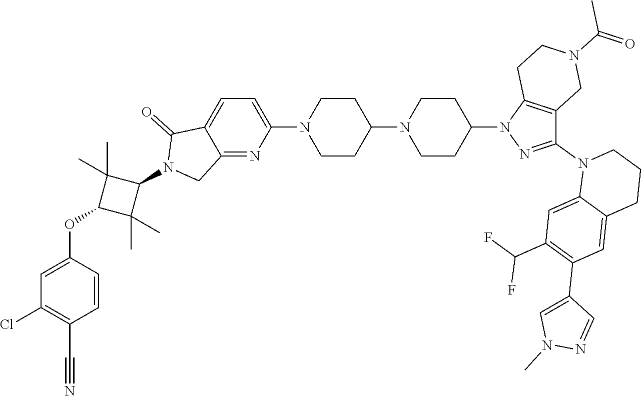 [SMILES: CC(=O)N1CCc2c(c(N3CCCc4cc(-c5cnn(C)c5)c(C(F)F)cc43)nn2C2CCN(C3CCN(c4ccc5c(n4)CN([C@H]4C(C)(C)[C@H](Oc6ccc(C#N)c(Cl)c6)C4(C)C)C5=O)CC3)CC2)C1] |
| 298 | 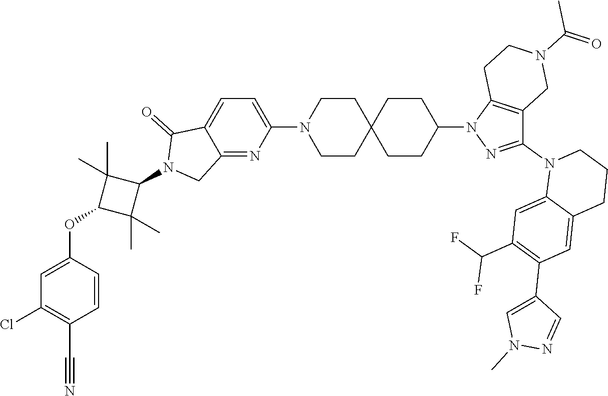 [SMILES: CC(=O)N1CCc2c(c(N3CCCc4cc(-c5cnn(C)c5)c(C(F)F)cc43)nn2C2CCC3(CC2)CCN(c2ccc4c(n2)CN([C@H]2C(C)(C)[C@H](Oc5ccc(C#N)c(Cl)c5)C2(C)C)C4=O)CC3)C1] |
| 299 | 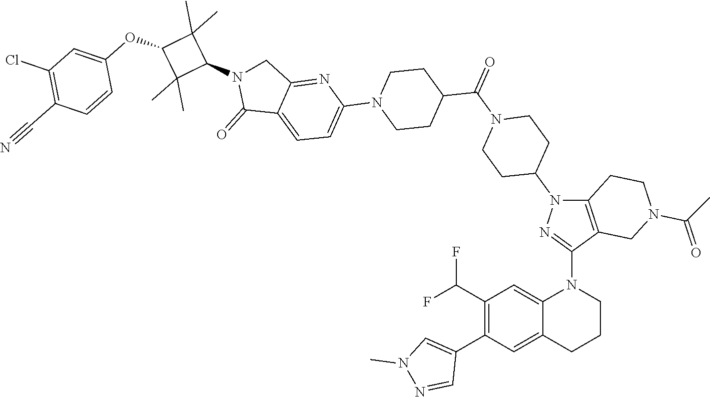 [SMILES: CC(=O)N1CCc2c(c(N3CCCc4cc(-c5cnn(C)c5)c(C(F)F)cc43)nn2C2CCN(C(=O)C3CCN(c4ccc5c(n4)CN([C@H]4C(C)(C)[C@H](Oc6ccc(C#N)c(Cl)c6)C4(C)C)C5=O)CC3)CC2)C1] |
| 300 | 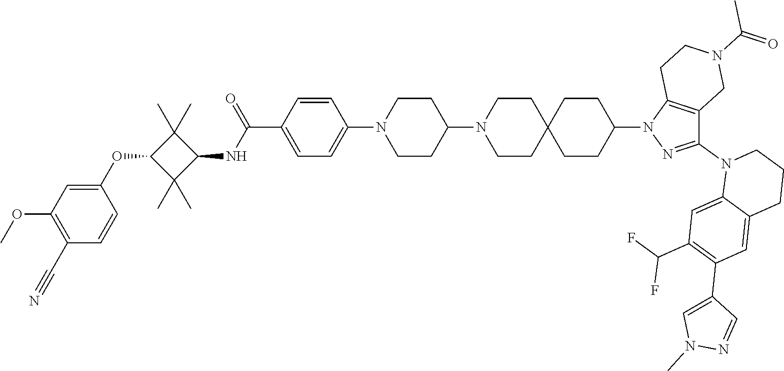 [SMILES: COc1cc(O[C@H]2C(C)(C)[C@H](NC(=O)c3ccc(N4CCC(N5CCC6(CCC(n7nc(N8CCCc9cc(-c%10cnn(C)c%10)c(C(F)F)cc98)c8c7CCN(C(C)=O)C8)CC6)CC5)CC4)cc3)C2(C)C)ccc1C#N] |
| 301 | 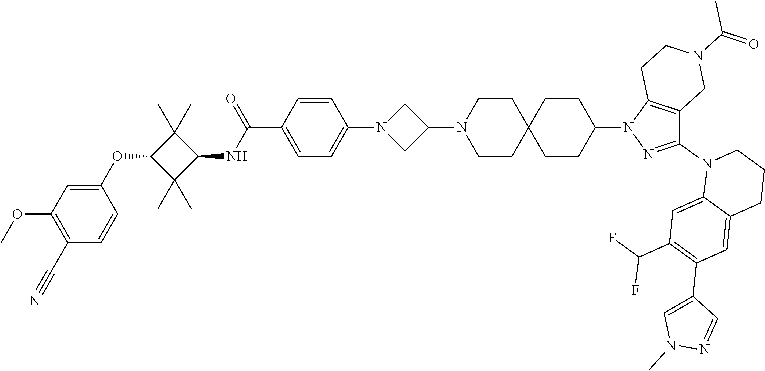 [SMILES: COc1cc(O[C@H]2C(C)(C)[C@H](NC(=O)c3ccc(N4CC(N5CCC6(CCC(n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 302 | 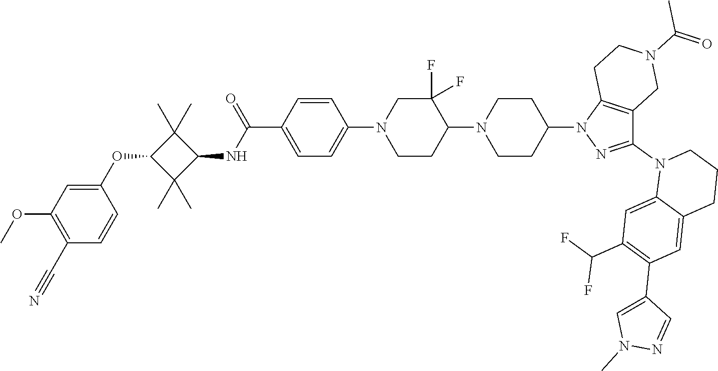 [SMILES: COc1cc(O[C@H]2C(C)(C)[C@H](NC(=O)c3ccc(N4CCC(N5CCC(n6nc(N7CCCc8cc(-c9cnn(C)c9)c(C(F)F)cc87)c7c6CCN(C(C)=O)C7)CC5)C(F)(F)C4)cc3)C2(C)C)ccc1C#N] |
| 303 | 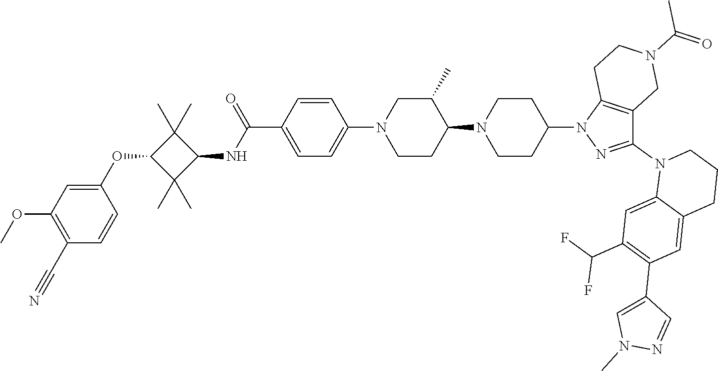 [SMILES: COc1cc(O[C@H]2C(C)(C)[C@H](NC(=O)c3ccc(N4CC[C@H](N5CCC(n6nc(N7CCCc8cc(-c9cnn(C)c9)c(C(F)F)cc87)c7c6CCN(C(C)=O)C7)CC5)[C@@H](C)C4)cc3)C2(C)C)ccc1C#N] |
| 304 | 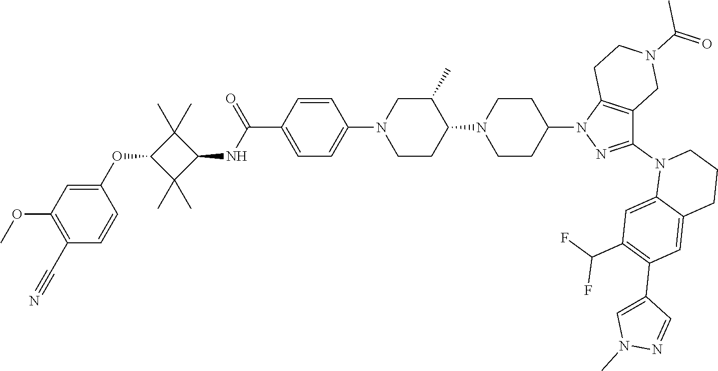 [SMILES: COc1cc(O[C@H]2C(C)(C)[C@H](NC(=O)c3ccc(N4CC[C@@H](N5CCC(n6nc(N7CCCc8cc(-c9cnn(C)c9)c(C(F)F)cc87)c7c6CCN(C(C)=O)C7)CC5)[C@@H](C)C4)cc3)C2(C)C)ccc1C#N] |
| 305 | 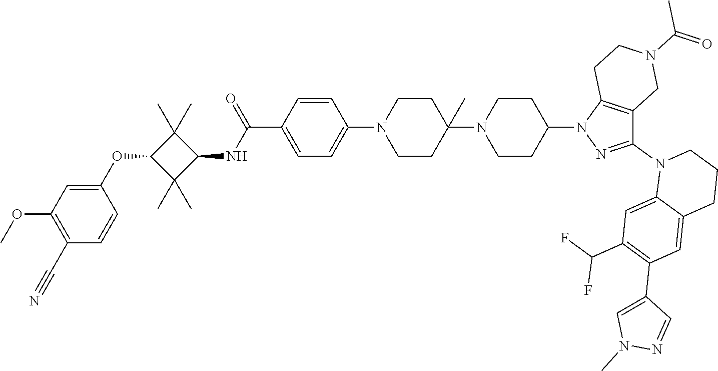 [SMILES: COc1cc(O[C@H]2C(C)(C)[C@H](NC(=O)c3ccc(N4CCC(C)(N5CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 306 | 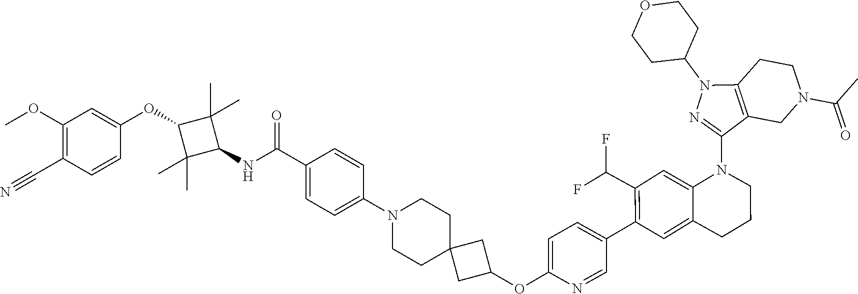 [SMILES: COc1cc(O[C@H]2C(C)(C)[C@H](NC(=O)c3ccc(N4CCC5(CC4)CC(Oc4ccc(-c6cc7c(cc6C(F)F)N(c6nn(C8CCOCC8)c8c6CN(C(C)=O)CC8)CCC7)cn4)C5)cc3)C2(C)C)ccc1C#N] |
| 307 | 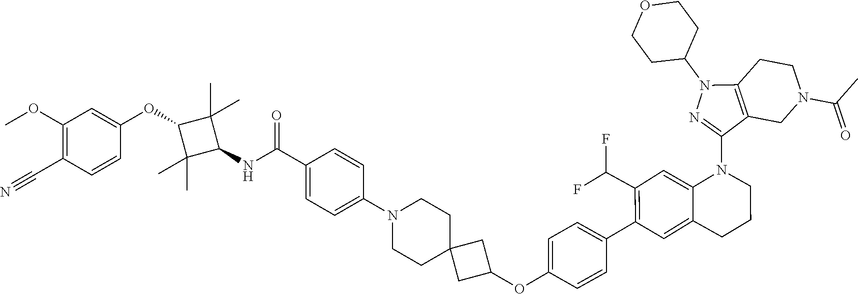 [SMILES: COc1cc(O[C@H]2C(C)(C)[C@H](NC(=O)c3ccc(N4CCC5(CC4)CC(Oc4ccc(-c6cc7c(cc6C(F)F)N(c6nn(C8CCOCC8)c8c6CN(C(C)=O)CC8)CCC7)cc4)C5)cc3)C2(C)C)ccc1C#N] |
| 308 | 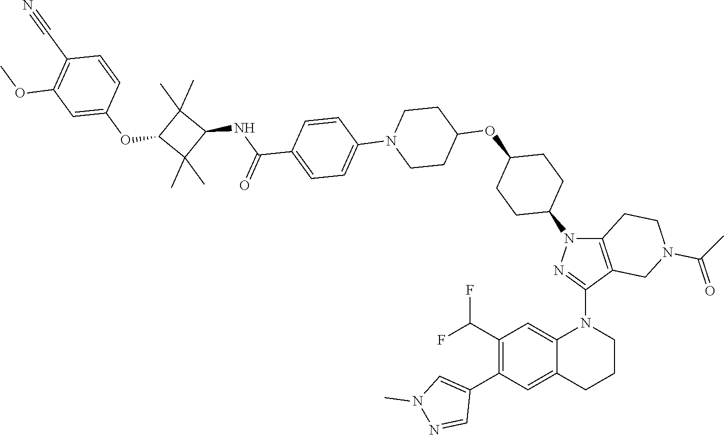 [SMILES: COc1cc(O[C@H]2C(C)(C)[C@H](NC(=O)c3ccc(N4CCC(O[C@H]5CC[C@@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 309 | 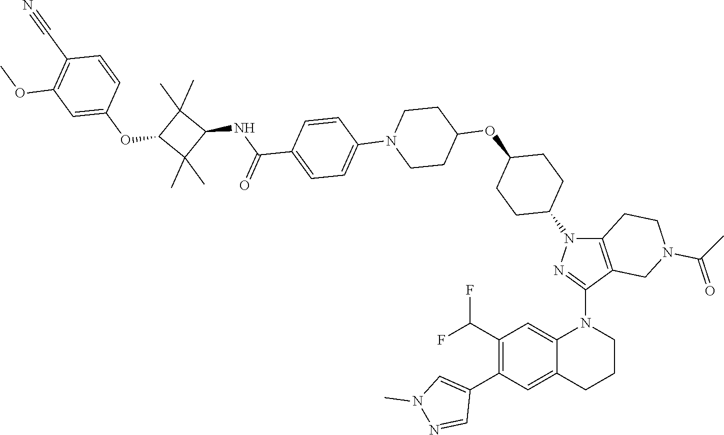 [SMILES: COc1cc(O[C@H]2C(C)(C)[C@H](NC(=O)c3ccc(N4CCC(O[C@H]5CC[C@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 310 | 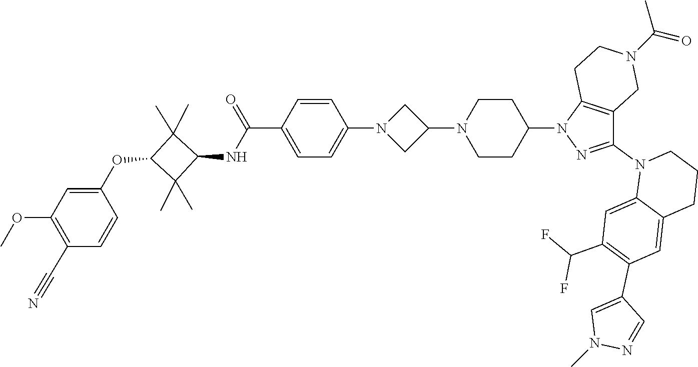 [SMILES: COc1cc(O[C@H]2C(C)(C)[C@H](NC(=O)c3ccc(N4CC(N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 311 | 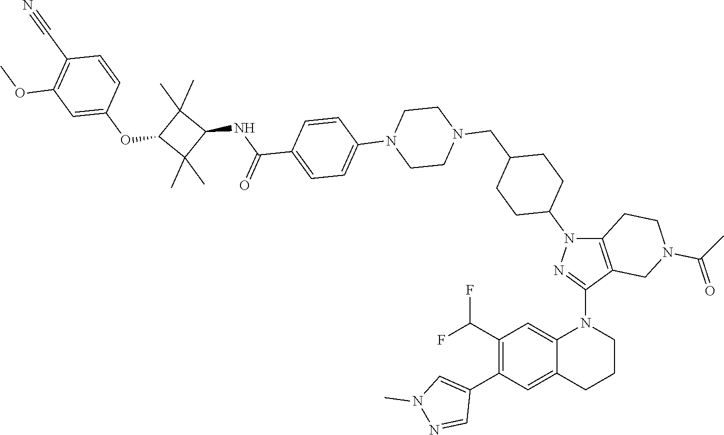 [SMILES: COc1cc(O[C@H]2C(C)(C)[C@H](NC(=O)c3ccc(N4CCN(CC5CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 312 | 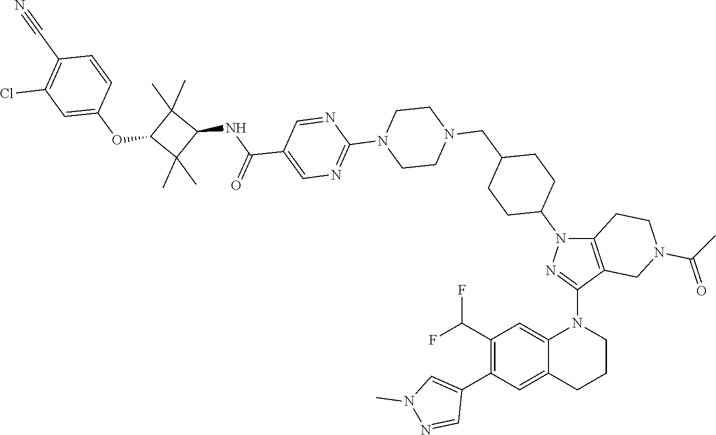 [SMILES: CC(=O)N1CCc2c(c(N3CCCc4cc(-c5cnn(C)c5)c(C(F)F)cc43)nn2C2CCC(CN3CCN(c4ncc(C(=O)N[C@H]5C(C)(C)[C@H](Oc6ccc(C#N)c(Cl)c6)C5(C)C)cn4)CC3)CC2)C1] |
| 313 | 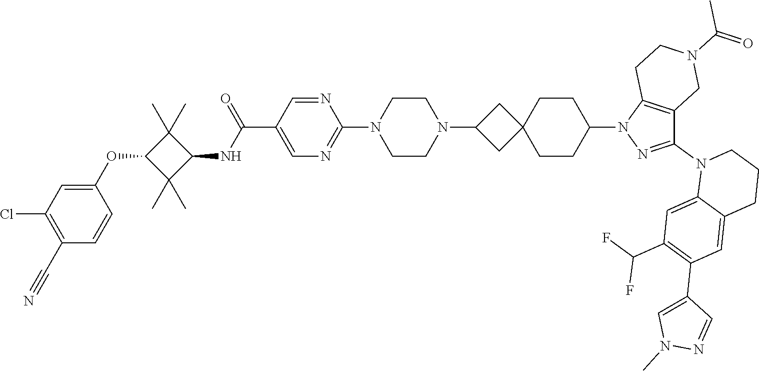 [SMILES: CC(=O)N1CCc2c(c(N3CCCc4cc(-c5cnn(C)c5)c(C(F)F)cc43)nn2C2CCC3(CC2)CC(N2CCN(c4ncc(C(=O)N[C@H]5C(C)(C)[C@H](Oc6ccc(C#N)c(Cl)c6)C5(C)C)cn4)CC2)C3)C1] |
| 314 | 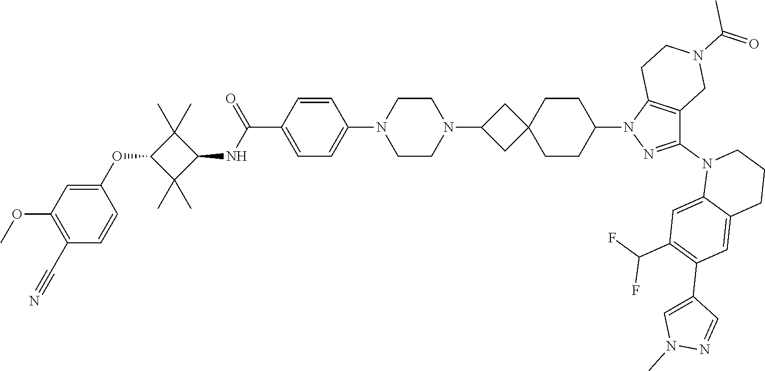 [SMILES: COc1cc(O[C@H]2C(C)(C)[C@H](NC(=O)c3ccc(N4CCN(C5CC6(CCC(n7nc(N8CCCc9cc(-c%10cnn(C)c%10)c(C(F)F)cc98)c8c7CCN(C(C)=O)C8)CC6)C5)CC4)cc3)C2(C)C)ccc1C#N] |
| 315 | 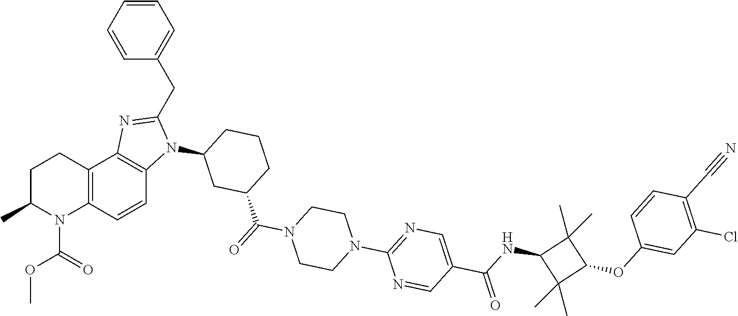 [SMILES: COC(=O)N1c2ccc3c(nc(Cc4ccccc4)n3[C@H]3CCC[C@H](C(=O)N4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 316 | 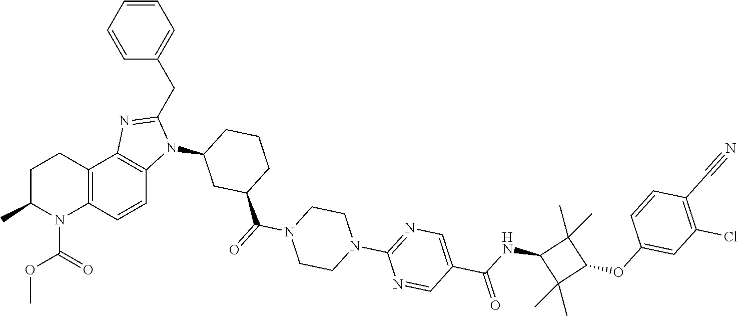 [SMILES: COC(=O)N1c2ccc3c(nc(Cc4ccccc4)n3[C@H]3CCC[C@@H](C(=O)N4CCN(c5ncc(C(=O)N[C@H]6C(C)(C)[C@H](Oc7ccc(C#N)c(Cl)c7)C6(C)C)cn5)CC4)C3)c2CC[C@@H]1C] |
| 317 | 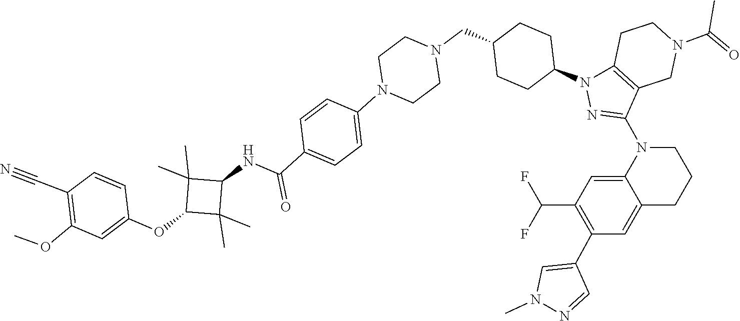 [SMILES: COc1cc(O[C@H]2C(C)(C)[C@H](NC(=O)c3ccc(N4CCN(C[C@H]5CC[C@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 318 | 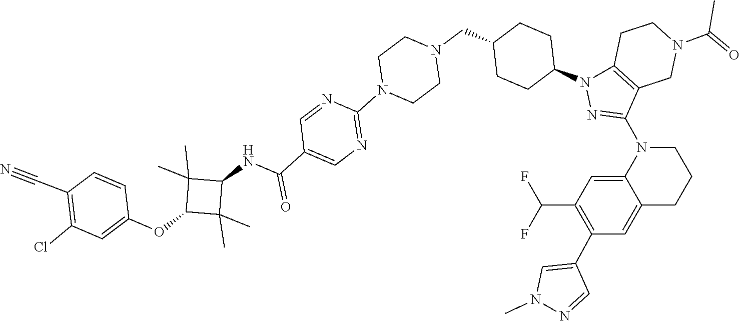 [SMILES: CC(=O)N1CCc2c(c(N3CCCc4cc(-c5cnn(C)c5)c(C(F)F)cc43)nn2[C@H]2CC[C@H](CN3CCN(c4ncc(C(=O)N[C@H]5C(C)(C)[C@H](Oc6ccc(C#N)c(Cl)c6)C5(C)C)cn4)CC3)CC2)C1] |
| 319 | 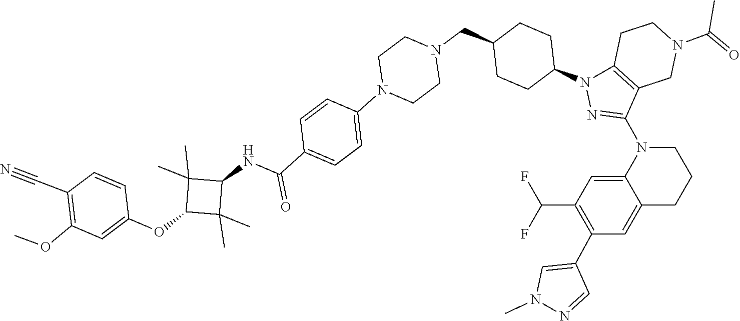 [SMILES: COc1cc(O[C@H]2C(C)(C)[C@H](NC(=O)c3ccc(N4CCN(C[C@H]5CC[C@@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 320 | 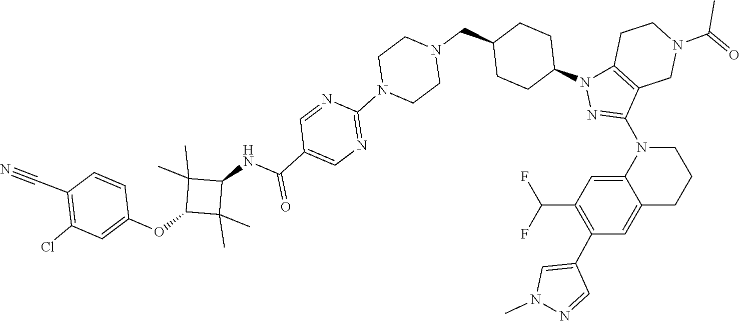 [SMILES: CC(=O)N1CCc2c(c(N3CCCc4cc(-c5cnn(C)c5)c(C(F)F)cc43)nn2[C@H]2CC[C@@H](CN3CCN(c4ncc(C(=O)N[C@H]5C(C)(C)[C@H](Oc6ccc(C#N)c(Cl)c6)C5(C)C)cn4)CC3)CC2)C1] |
| 321 | 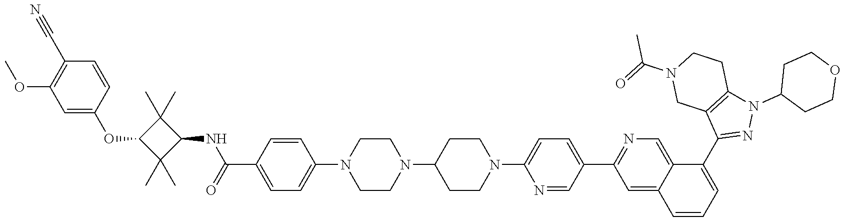 [SMILES: COc1cc(O[C@H]2C(C)(C)[C@H](NC(=O)c3ccc(N4CCN(C5CCN(c6ccc(-c7cc8cccc(-c9nn(C%10CCOCC%10)c%10c9CN(C(C)=O)CC%10)c8cn7)cn6)CC5)CC4)cc3)C2(C)C)ccc1C#N] |
| 322 | 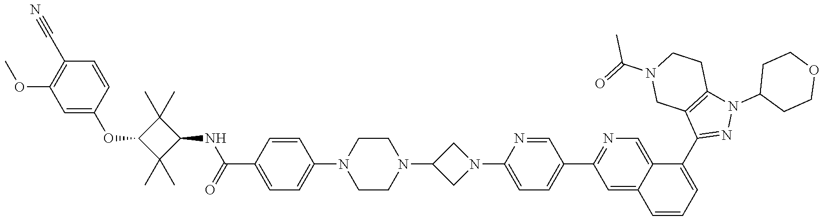 [SMILES: COc1cc(O[C@H]2C(C)(C)[C@H](NC(=O)c3ccc(N4CCN(C5CN(c6ccc(-c7cc8cccc(-c9nn(C%10CCOCC%10)c%10c9CN(C(C)=O)CC%10)c8cn7)cn6)C5)CC4)cc3)C2(C)C)ccc1C#N] |
| 323 | 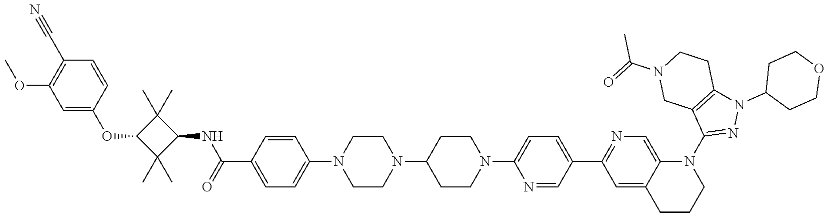 [SMILES: COc1cc(O[C@H]2C(C)(C)[C@H](NC(=O)c3ccc(N4CCN(C5CCN(c6ccc(-c7cc8c(cn7)N(c7nn(C9CCOCC9)c9c7CN(C(C)=O)CC9)CCC8)cn6)CC5)CC4)cc3)C2(C)C)ccc1C#N] |
| 324 | 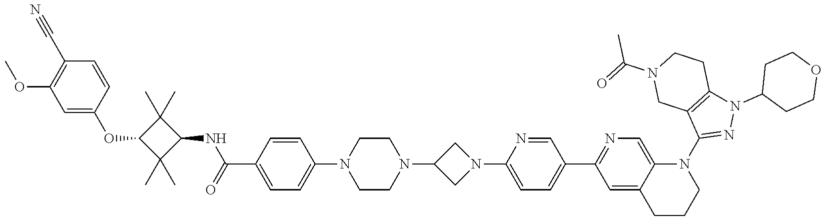 [SMILES: COc1cc(O[C@H]2C(C)(C)[C@H](NC(=O)c3ccc(N4CCN(C5CN(c6ccc(-c7cc8c(cn7)N(c7nn(C9CCOCC9)c9c7CN(C(C)=O)CC9)CCC8)cn6)C5)CC4)cc3)C2(C)C)ccc1C#N] |
| 325 | 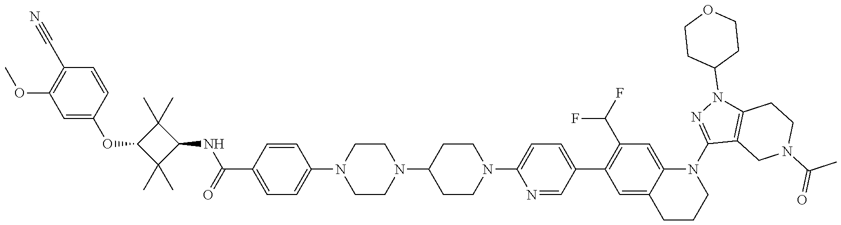 [SMILES: COc1cc(O[C@H]2C(C)(C)[C@H](NC(=O)c3ccc(N4CCN(C5CCN(c6ccc(-c7cc8c(cc7C(F)F)N(c7nn(C9CCOCC9)c9c7CN(C(C)=O)CC9)CCC8)cn6)CC5)CC4)cc3)C2(C)C)ccc1C#N] |
| 326 | 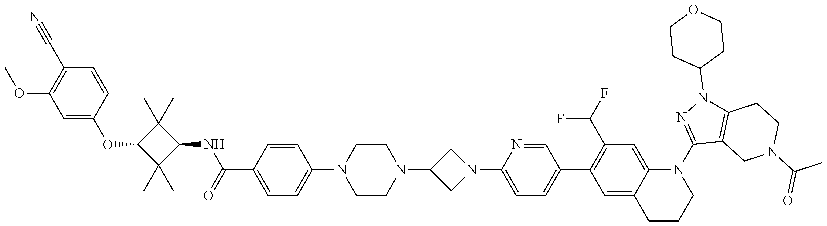 [SMILES: COc1cc(O[C@H]2C(C)(C)[C@H](NC(=O)c3ccc(N4CCN(C5CN(c6ccc(-c7cc8c(cc7C(F)F)N(c7nn(C9CCOCC9)c9c7CN(C(C)=O)CC9)CCC8)cn6)C5)CC4)cc3)C2(C)C)ccc1C#N] |
| 327 | 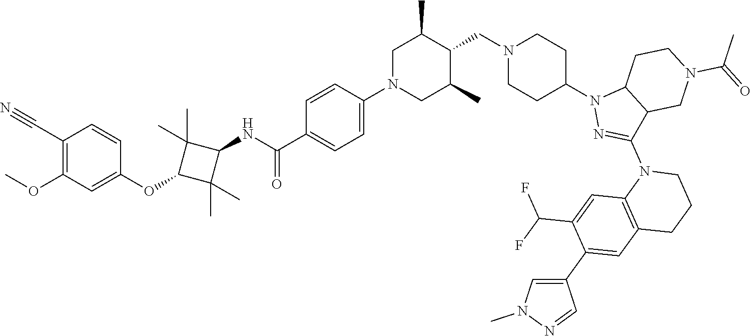 [SMILES: COc1cc(O[C@H]2C(C)(C)[C@H](NC(=O)c3ccc(N4C[C@@H](C)[C@H](CN5CCC(N6N=C(N7CCCc8cc(-c9cnn(C)c9)c(C(F)F)cc87)C7CN(C(C)=O)CCC76)CC5)[C@@H](C)C4)cc3)C2(C)C)ccc1C#N] |
| 328 | 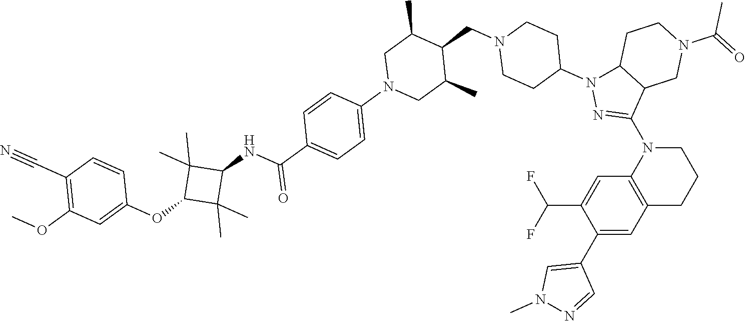 [SMILES: COc1cc(O[C@H]2C(C)(C)[C@H](NC(=O)c3ccc(N4C[C@@H](C)[C@@H](CN5CCC(N6N=C(N7CCCc8cc(-c9cnn(C)c9)c(C(F)F)cc87)C7CN(C(C)=O)CCC76)CC5)[C@@H](C)C4)cc3)C2(C)C)ccc1C#N] |
| 329 | 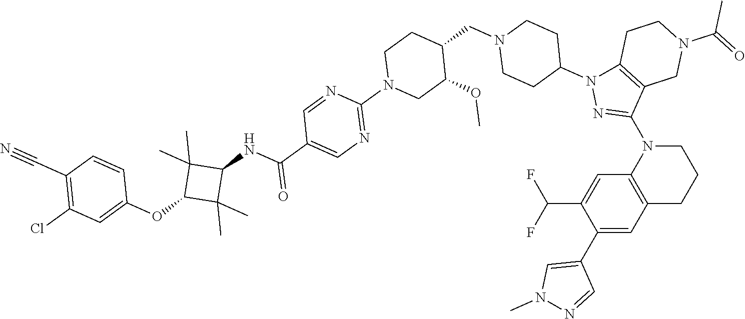 [SMILES: CO[C@@H]1CN(c2ncc(C(=O)N[C@H]3C(C)(C)[C@H](Oc4ccc(C#N)c(Cl)c4)C3(C)C)cn2)CC[C@@H]1CN1CCC(n2nc(N3CCCc4cc(-c5cnn(C)c5)c(C(F)F)cc43)c3c2CCN(C(C)=O)C3)CC1] |
| 330 | 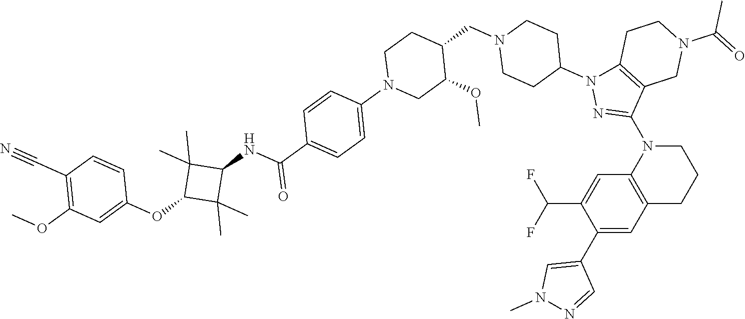 [SMILES: COc1cc(O[C@H]2C(C)(C)[C@H](NC(=O)c3ccc(N4CC[C@H](CN5CCC(n6nc(N7CCCc8cc(-c9cnn(C)c9)c(C(F)F)cc87)c7c6CCN(C(C)=O)C7)CC5)[C@H](OC)C4)cc3)C2(C)C)ccc1C#N] |
| 331 | 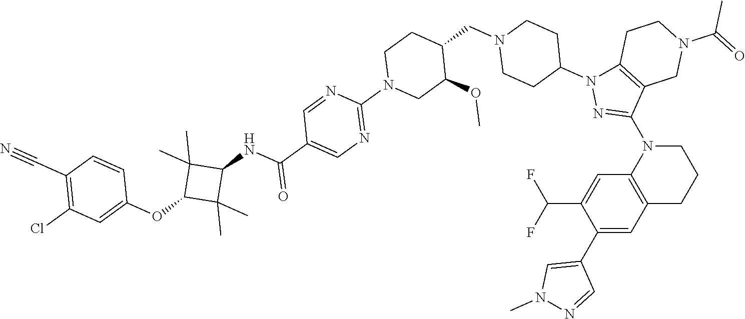 [SMILES: CO[C@H]1CN(c2ncc(C(=O)N[C@H]3C(C)(C)[C@H](Oc4ccc(C#N)c(Cl)c4)C3(C)C)cn2)CC[C@@H]1CN1CCC(n2nc(N3CCCc4cc(-c5cnn(C)c5)c(C(F)F)cc43)c3c2CCN(C(C)=O)C3)CC1] |
| 332 | 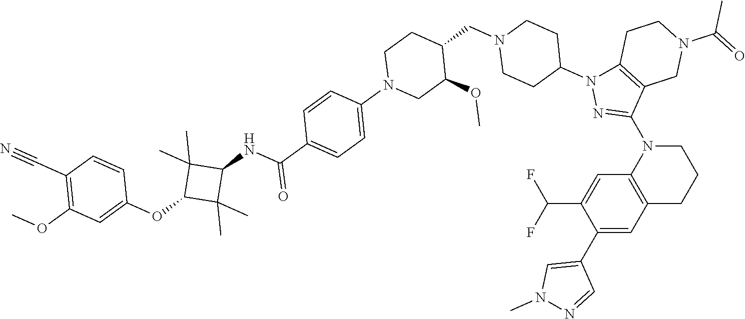 [SMILES: COc1cc(O[C@H]2C(C)(C)[C@H](NC(=O)c3ccc(N4CC[C@H](CN5CCC(n6nc(N7CCCc8cc(-c9cnn(C)c9)c(C(F)F)cc87)c7c6CCN(C(C)=O)C7)CC5)[C@@H](OC)C4)cc3)C2(C)C)ccc1C#N] |
| 333 | 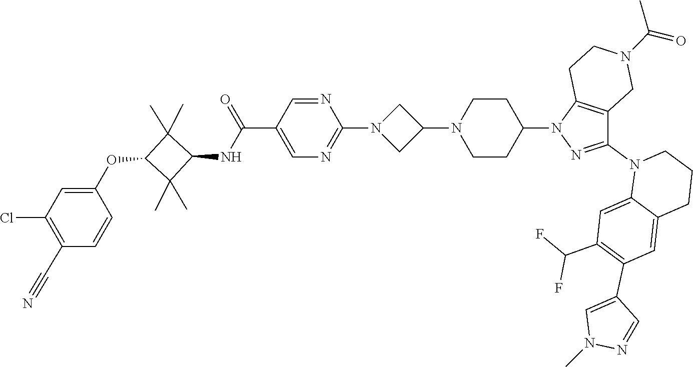 [SMILES: CC(=O)N1CCc2c(c(N3CCCc4cc(-c5cnn(C)c5)c(C(F)F)cc43)nn2C2CCN(C3CN(c4ncc(C(=O)N[C@H]5C(C)(C)[C@H](Oc6ccc(C#N)c(Cl)c6)C5(C)C)cn4)C3)CC2)C1] |
| 334 | 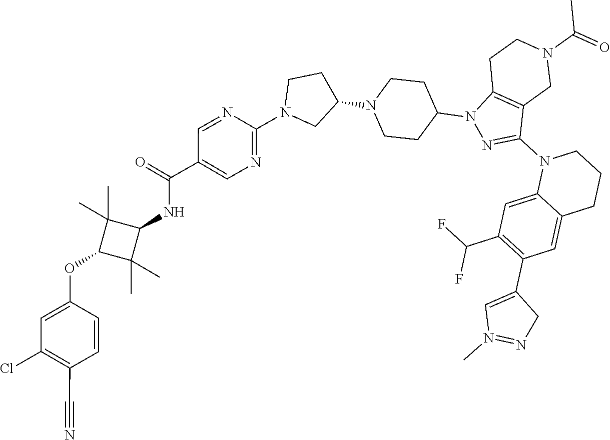 |
| 335 | 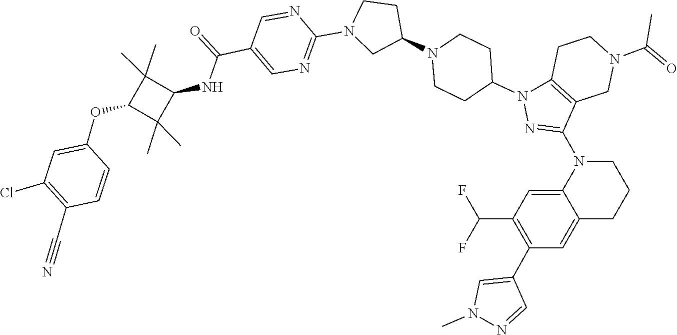 [SMILES: CC(=O)N1CCc2c(c(N3CCCc4cc(-c5cnn(C)c5)c(C(F)F)cc43)nn2C2CCN([C@@H]3CCN(c4ncc(C(=O)N[C@H]5C(C)(C)[C@H](Oc6ccc(C#N)c(Cl)c6)C5(C)C)cn4)C3)CC2)C1] |
| 336 | 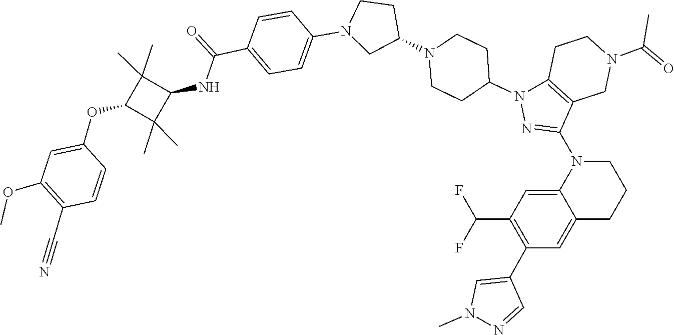 [SMILES: COc1cc(O[C@H]2C(C)(C)[C@H](NC(=O)c3ccc(N4CC[C@H](N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 337 | 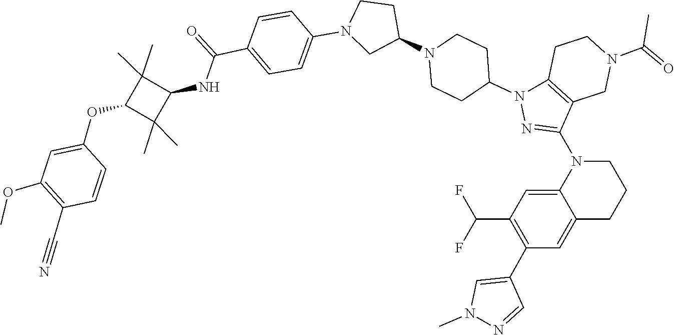 [SMILES: COc1cc(O[C@H]2C(C)(C)[C@H](NC(=O)c3ccc(N4CC[C@@H](N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 338 | 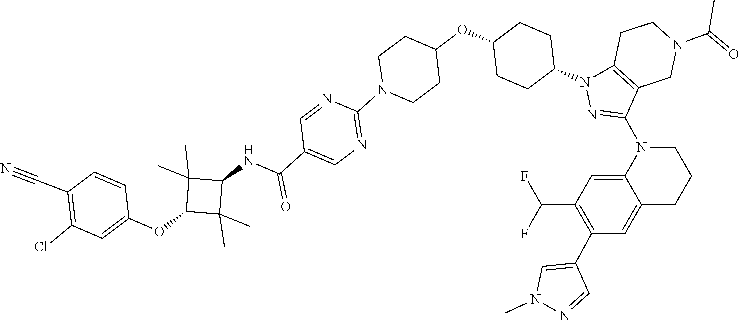 [SMILES: CC(=O)N1CCc2c(c(N3CCCc4cc(-c5cnn(C)c5)c(C(F)F)cc43)nn2[C@H]2CC[C@@H](OC3CCN(c4ncc(C(=O)N[C@H]5C(C)(C)[C@H](Oc6ccc(C#N)c(Cl)c6)C5(C)C)cn4)CC3)CC2)C1] |
| 339 | 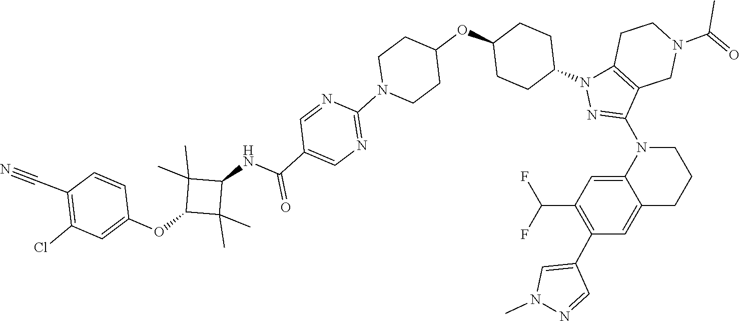 [SMILES: CC(=O)N1CCc2c(c(N3CCCc4cc(-c5cnn(C)c5)c(C(F)F)cc43)nn2[C@H]2CC[C@H](OC3CCN(c4ncc(C(=O)N[C@H]5C(C)(C)[C@H](Oc6ccc(C#N)c(Cl)c6)C5(C)C)cn4)CC3)CC2)C1] |
| 340 | 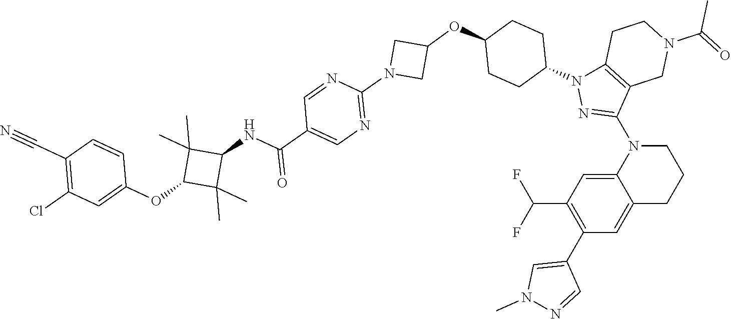 [SMILES: CC(=O)N1CCc2c(c(N3CCCc4cc(-c5cnn(C)c5)c(C(F)F)cc43)nn2[C@H]2CC[C@H](OC3CN(c4ncc(C(=O)N[C@H]5C(C)(C)[C@H](Oc6ccc(C#N)c(Cl)c6)C5(C)C)cn4)C3)CC2)C1] |
| 341 | 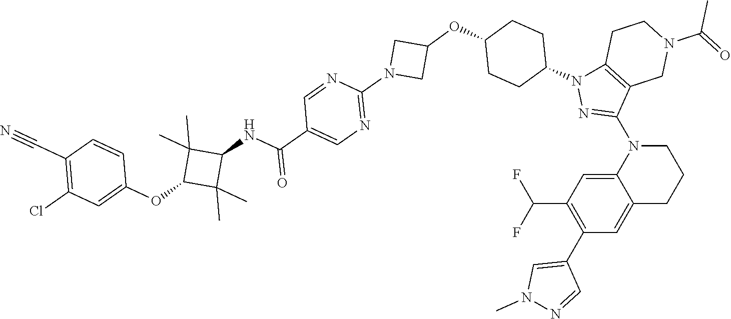 [SMILES: CC(=O)N1CCc2c(c(N3CCCc4cc(-c5cnn(C)c5)c(C(F)F)cc43)nn2[C@H]2CC[C@@H](OC3CN(c4ncc(C(=O)N[C@H]5C(C)(C)[C@H](Oc6ccc(C#N)c(Cl)c6)C5(C)C)cn4)C3)CC2)C1] |
| 342 | 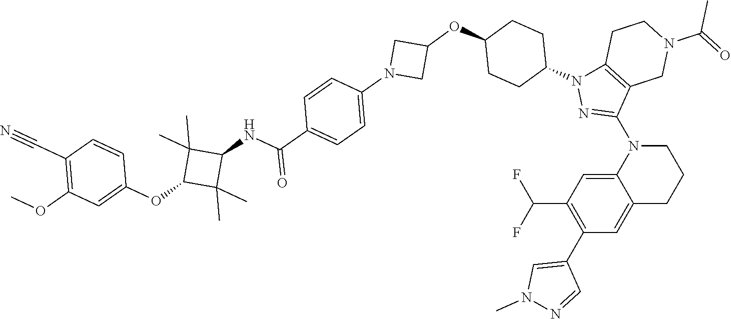 [SMILES: COc1cc(O[C@H]2C(C)(C)[C@H](NC(=O)c3ccc(N4CC(O[C@H]5CC[C@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 343 | 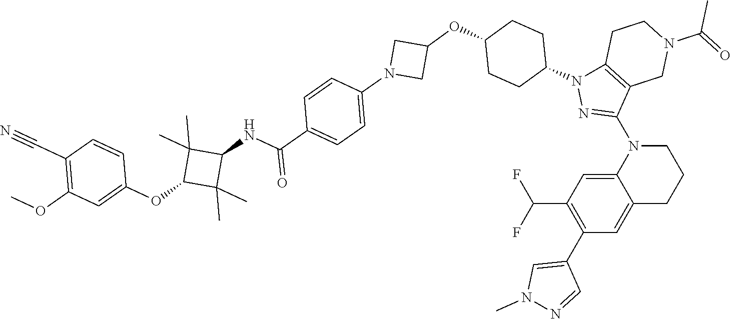 [SMILES: COc1cc(O[C@H]2C(C)(C)[C@H](NC(=O)c3ccc(N4CC(O[C@H]5CC[C@@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 344 | 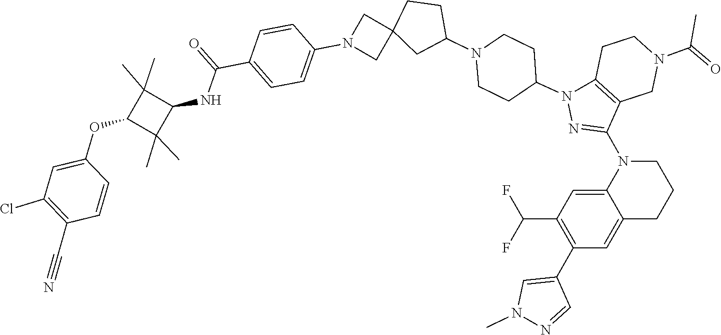 [SMILES: CC(=O)N1CCc2c(c(N3CCCc4cc(-c5cnn(C)c5)c(C(F)F)cc43)nn2C2CCN(C3CCC4(C3)CN(c3ccc(C(=O)N[C@H]5C(C)(C)[C@H](Oc6ccc(C#N)c(Cl)c6)C5(C)C)cc3)C4)CC2)C1] |
| 345 | 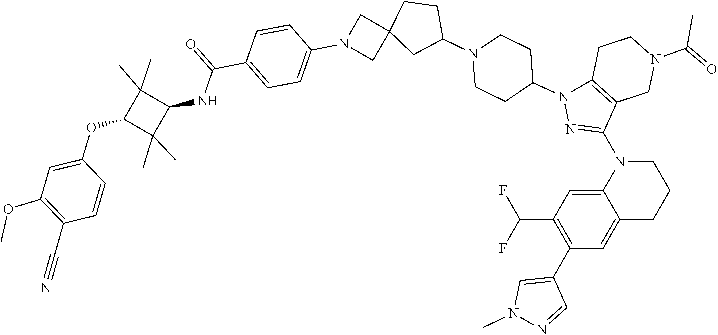 [SMILES: COc1cc(O[C@H]2C(C)(C)[C@H](NC(=O)c3ccc(N4CC5(CCC(N6CCC(n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 346 | 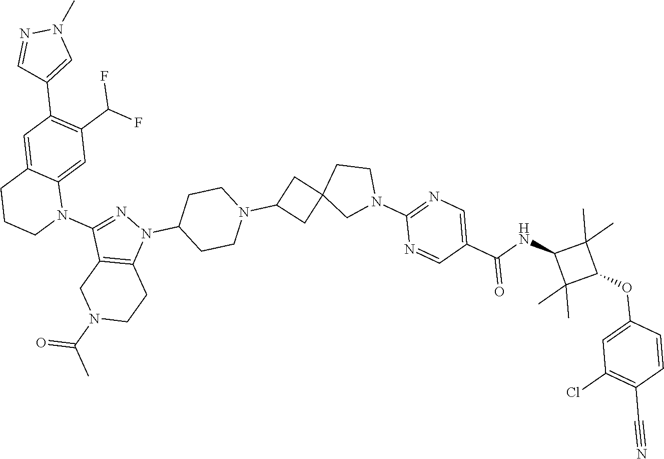 [SMILES: CC(=O)N1CCc2c(c(N3CCCc4cc(-c5cnn(C)c5)c(C(F)F)cc43)nn2C2CCN(C3CC4(CCN(c5ncc(C(=O)N[C@H]6C(C)(C)[C@H](Oc7ccc(C#N)c(Cl)c7)C6(C)C)cn5)C4)C3)CC2)C1] |
| 347 | 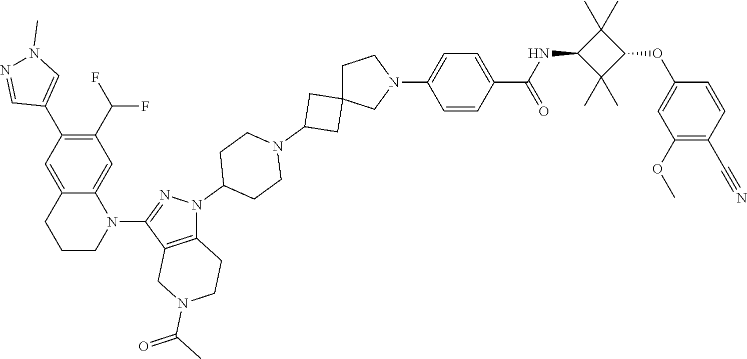 [SMILES: COc1cc(O[C@H]2C(C)(C)[C@H](NC(=O)c3ccc(N4CCC5(CC(N6CCC(n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 348 | 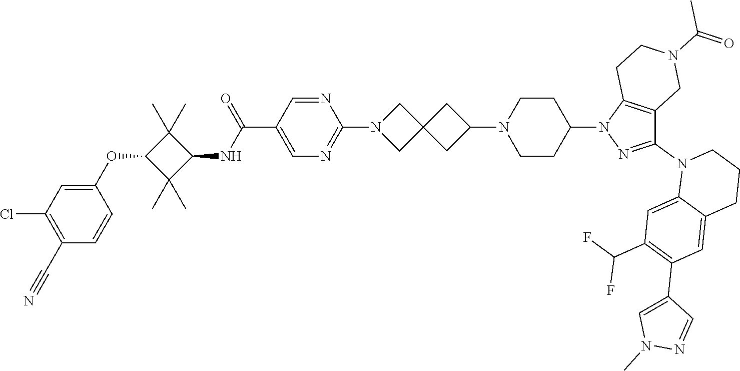 [SMILES: CC(=O)N1CCc2c(c(N3CCCc4cc(-c5cnn(C)c5)c(C(F)F)cc43)nn2C2CCN(C3CC4(C3)CN(c3ncc(C(=O)N[C@H]5C(C)(C)[C@H](Oc6ccc(C#N)c(Cl)c6)C5(C)C)cn3)C4)CC2)C1] |
| 349 | 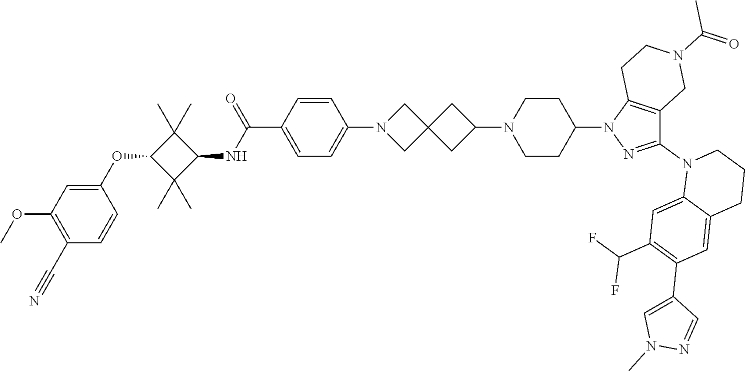 [SMILES: COc1cc(O[C@H]2C(C)(C)[C@H](NC(=O)c3ccc(N4CC5(CC(N6CCC(n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 350 | 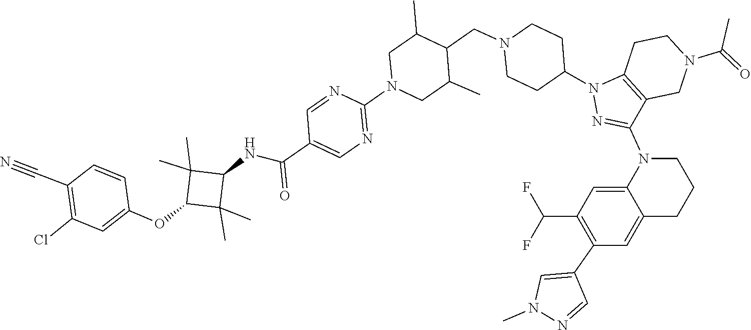 [SMILES: CC(=O)N1CCc2c(c(N3CCCc4cc(-c5cnn(C)c5)c(C(F)F)cc43)nn2C2CCN(CC3C(C)CN(c4ncc(C(=O)N[C@H]5C(C)(C)[C@H](Oc6ccc(C#N)c(Cl)c6)C5(C)C)cn4)CC3C)CC2)C1] |
| 355 | 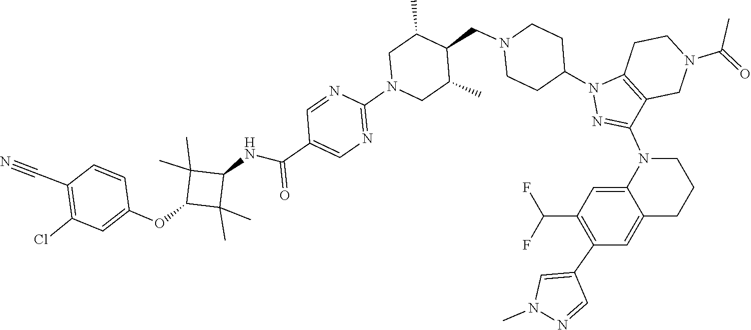 [SMILES: CC(=O)N1CCc2c(c(N3CCCc4cc(-c5cnn(C)c5)c(C(F)F)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 356 | 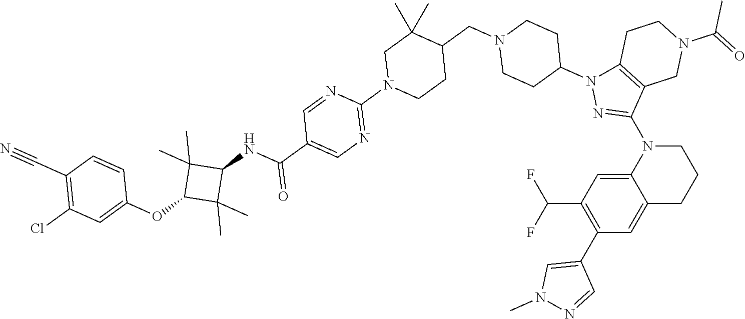 [SMILES: CC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3(C)C)CC2)C1] |
| 357 | 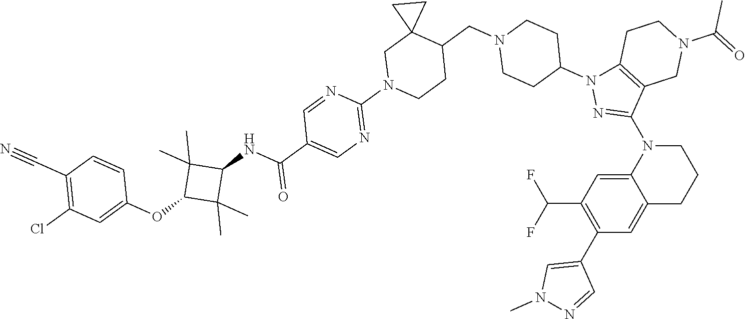 [SMILES: CC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC34CC4)CC2)C1] |
| 360 | 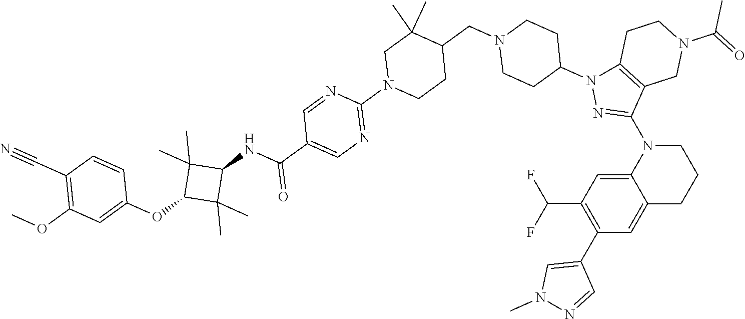 [SMILES: COc1cc(O[C@H]2C(C)(C)[C@H](NC(=O)c3cnc(N4CCC(CN5CCC(n6nc(N7CCCc8cc(-c9cnn(C)c9)c(C(F)F)cc87)c7c6CCN(C(C)=O)C7)CC5)C(C)(C)C4)nc3)C2(C)C)ccc1C#N] |
| 362 | 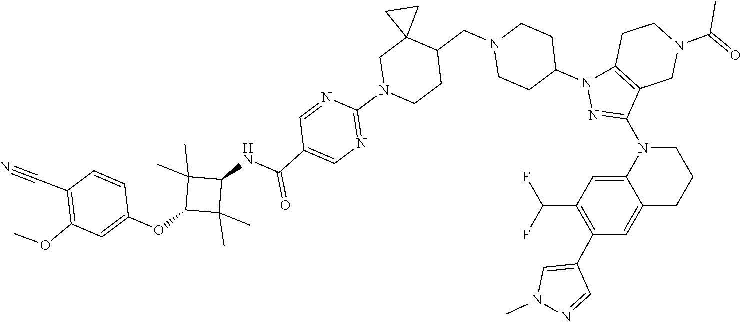 [SMILES: COc1cc(O[C@H]2C(C)(C)[C@H](NC(=O)c3cnc(N4CCC(CN5CCC(n6nc(N7CCCc8cc(-c9cnn(C)c9)c(C(F)F)cc87)c7c6CCN(C(C)=O)C7)CC5)C5(CC5)C4)nc3)C2(C)C)ccc1C#N] |
| 363 | 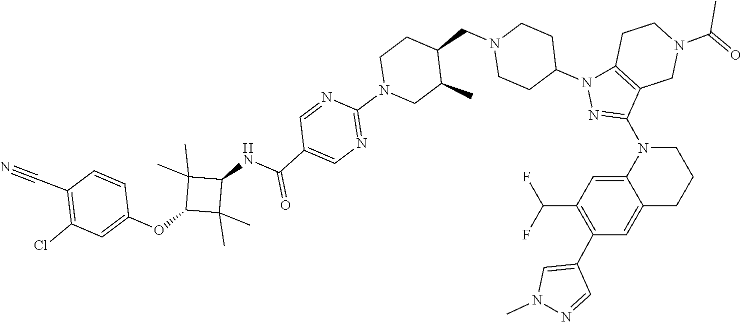 [SMILES: CC(=O)N1CCc2c(c(N3CCCc4cc(-c5cnn(C)c5)c(C(F)F)cc43)nn2C2CCN(C[C@@H]3CCN(c4ncc(C(=O)N[C@H]5C(C)(C)[C@H](Oc6ccc(C#N)c(Cl)c6)C5(C)C)cn4)C[C@@H]3C)CC2)C1] |
| 364 | 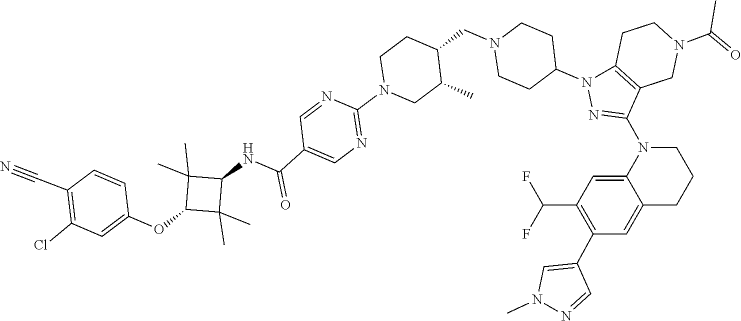 [SMILES: CC(=O)N1CCc2c(c(N3CCCc4cc(-c5cnn(C)c5)c(C(F)F)cc43)nn2C2CCN(C[C@H]3CCN(c4ncc(C(=O)N[C@H]5C(C)(C)[C@H](Oc6ccc(C#N)c(Cl)c6)C5(C)C)cn4)C[C@H]3C)CC2)C1] |
| 365 | 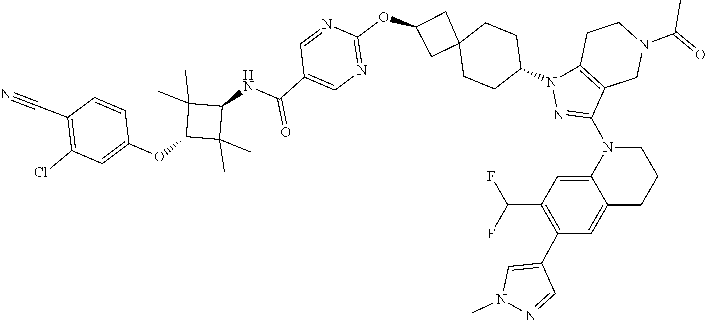 [SMILES: CC(=O)N1CCc2c(c(N3CCCc4cc(-c5cnn(C)c5)c(C(F)F)cc43)nn2[C@H]2CCC3(CC2)C[C@H](Oc2ncc(C(=O)N[C@H]4C(C)(C)[C@H](Oc5ccc(C#N)c(Cl)c5)C4(C)C)cn2)C3)C1] |
| 366 | 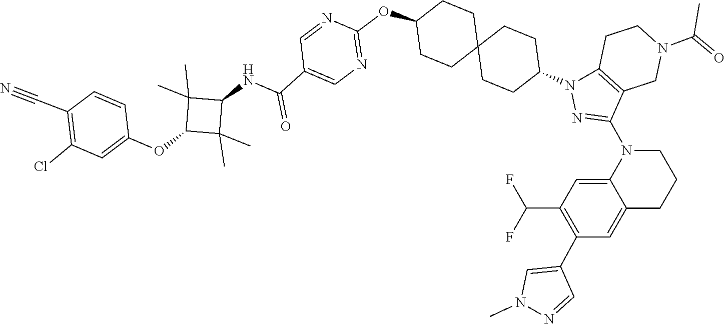 [SMILES: CC(=O)N1CCc2c(c(N3CCCc4cc(-c5cnn(C)c5)c(C(F)F)cc43)nn2[C@H]2CCC3(CC[C@H](Oc4ncc(C(=O)N[C@H]5C(C)(C)[C@H](Oc6ccc(C#N)c(Cl)c6)C5(C)C)cn4)CC3)CC2)C1] |
| 367 | 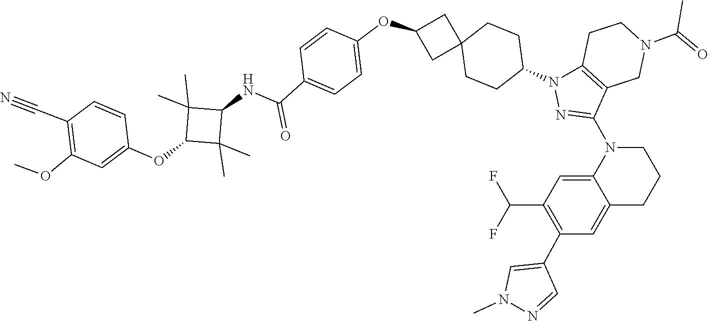 [SMILES: COc1cc(O[C@H]2C(C)(C)[C@H](NC(=O)c3ccc(O[C@H]4CC5(CC[C@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 368 | 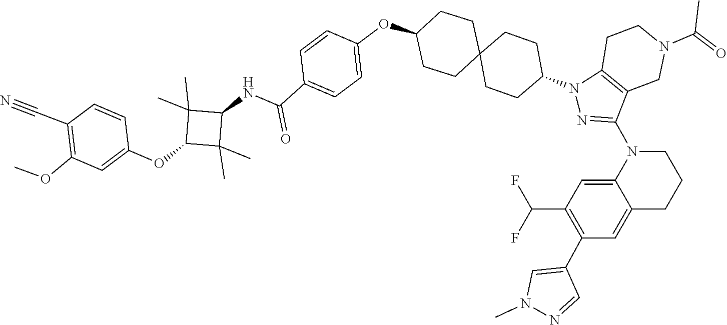 [SMILES: COc1cc(O[C@H]2C(C)(C)[C@H](NC(=O)c3ccc(O[C@H]4CCC5(CC4)CC[C@H](n4nc(N6CCCc7cc(-c8cnn(C)c8)c(C(F)F)cc76)c6c4CCN(C(C)=O)C6)CC5)cc3)C2(C)C)ccc1C#N] |
| 369 | 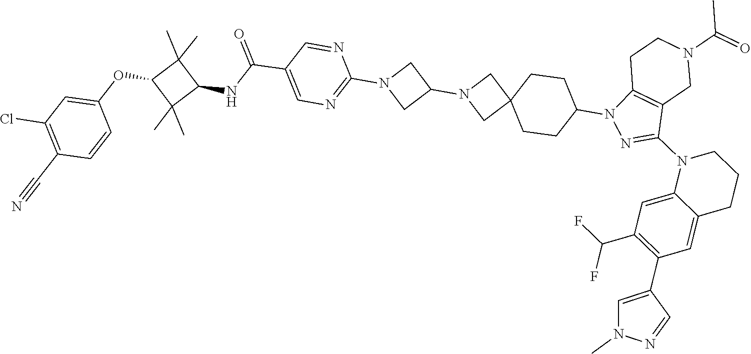 [SMILES: CC(=O)N1CCc2c(c(N3CCCc4cc(-c5cnn(C)c5)c(C(F)F)cc43)nn2C2CCC3(CC2)CN(C2CN(c4ncc(C(=O)N[C@H]5C(C)(C)[C@H](Oc6ccc(C#N)c(Cl)c6)C5(C)C)cn4)C2)C3)C1] |
| 370 | 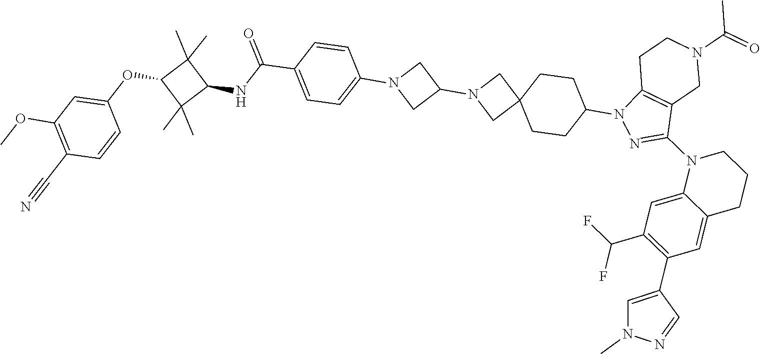 [SMILES: COc1cc(O[C@H]2C(C)(C)[C@H](NC(=O)c3ccc(N4CC(N5CC6(CCC(n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 371 | 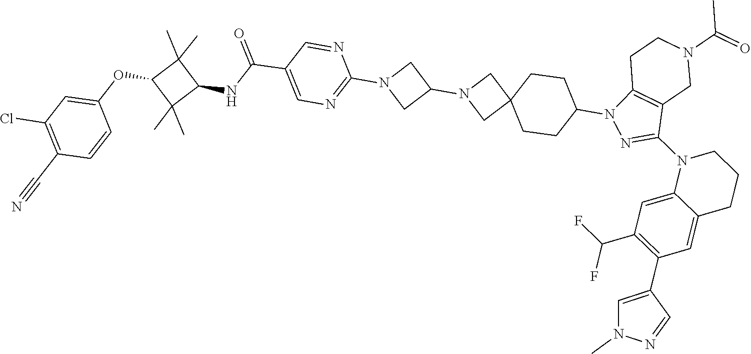 [SMILES: CC(=O)N1CCc2c(c(N3CCCc4cc(-c5cnn(C)c5)c(C(F)F)cc43)nn2C2CCC3(CC2)CN(C2CN(c4ncc(C(=O)N[C@H]5C(C)(C)[C@H](Oc6ccc(C#N)c(Cl)c6)C5(C)C)cn4)C2)C3)C1] |
| 372 | 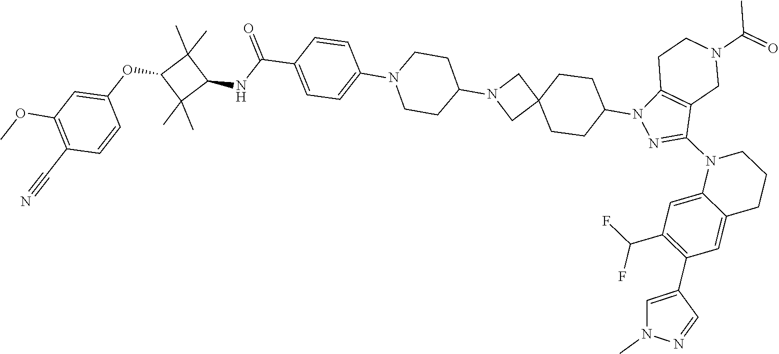 [SMILES: COc1cc(O[C@H]2C(C)(C)[C@H](NC(=O)c3ccc(N4CCC(N5CC6(CCC(n7nc(N8CCCc9cc(-c%10cnn(C)c%10)c(C(F)F)cc98)c8c7CCN(C(C)=O)C8)CC6)C5)CC4)cc3)C2(C)C)ccc1C#N] |
| 373 | 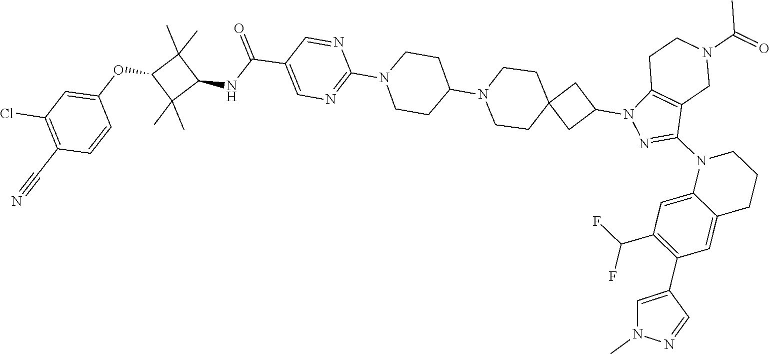 [SMILES: CC(=O)N1CCc2c(c(N3CCCc4cc(-c5cnn(C)c5)c(C(F)F)cc43)nn2C2CC3(CCN(C4CCN(c5ncc(C(=O)N[C@H]6C(C)(C)[C@H](Oc7ccc(C#N)c(Cl)c7)C6(C)C)cn5)CC4)CC3)C2)C1] |
| 374 | 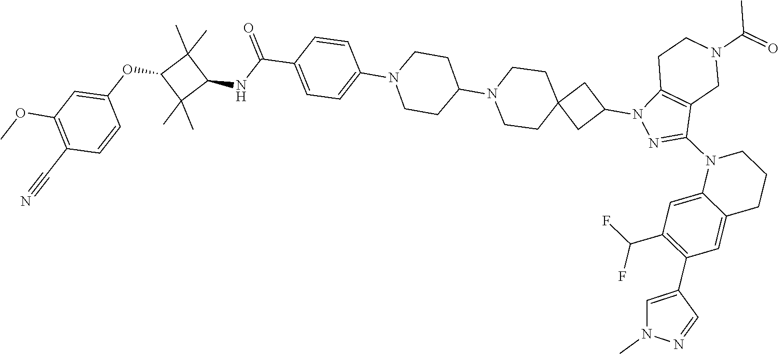 [SMILES: COc1cc(O[C@H]2C(C)(C)[C@H](NC(=O)c3ccc(N4CCC(N5CCC6(CC5)CC(n5nc(N7CCCc8cc(-c9cnn(C)c9)c(C(F)F)cc87)c7c5CCN(C(C)=O)C7)C6)CC4)cc3)C2(C)C)ccc1C#N] |
| 375 | 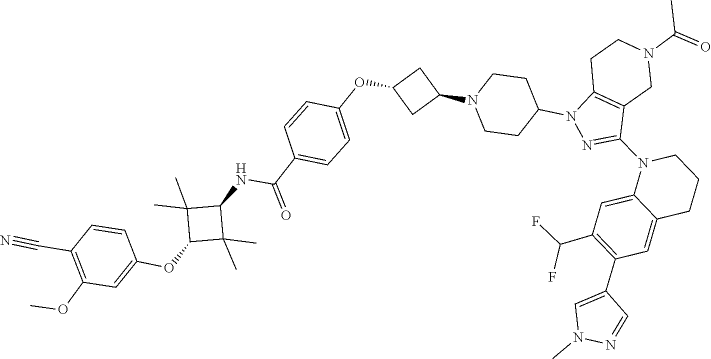 [SMILES: COc1cc(O[C@H]2C(C)(C)[C@H](NC(=O)c3ccc(O[C@H]4C[C@H](N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 376 | 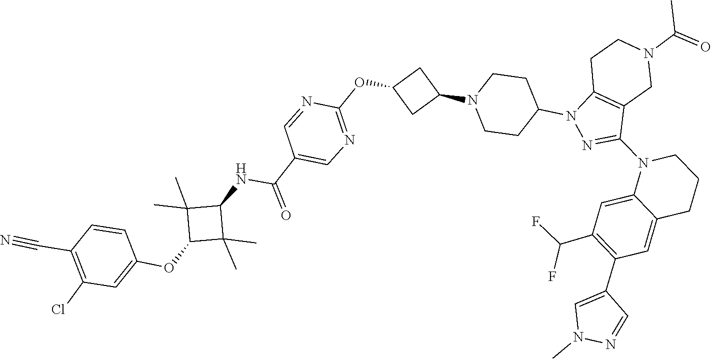 [SMILES: CC(=O)N1CCc2c(c(N3CCCc4cc(-c5cnn(C)c5)c(C(F)F)cc43)nn2C2CCN([C@H]3C[C@H](Oc4ncc(C(=O)N[C@H]5C(C)(C)[C@H](Oc6ccc(C#N)c(Cl)c6)C5(C)C)cn4)C3)CC2)C1] |
| 377 | 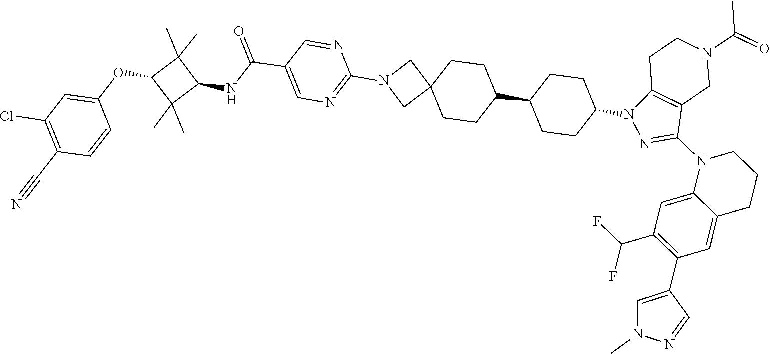 [SMILES: CC(=O)N1CCc2c(c(N3CCCc4cc(-c5cnn(C)c5)c(C(F)F)cc43)nn2[C@H]2CC[C@H](C3CCC4(CC3)CN(c3ncc(C(=O)N[C@H]5C(C)(C)[C@H](Oc6ccc(C#N)c(Cl)c6)C5(C)C)cn3)C4)CC2)C1] |
| 378 | 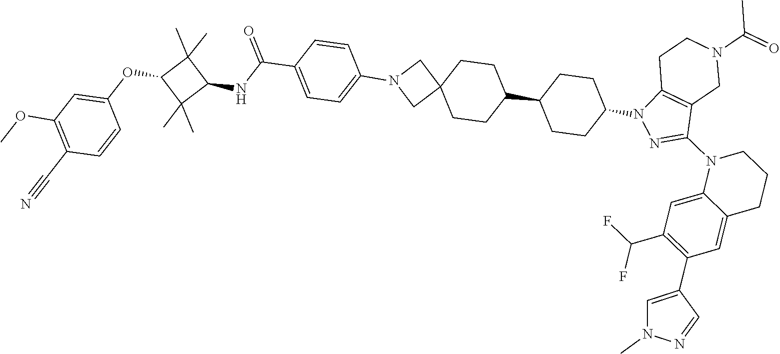 [SMILES: COc1cc(O[C@H]2C(C)(C)[C@H](NC(=O)c3ccc(N4CC5(CCC([C@H]6CC[C@H](n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 379 | 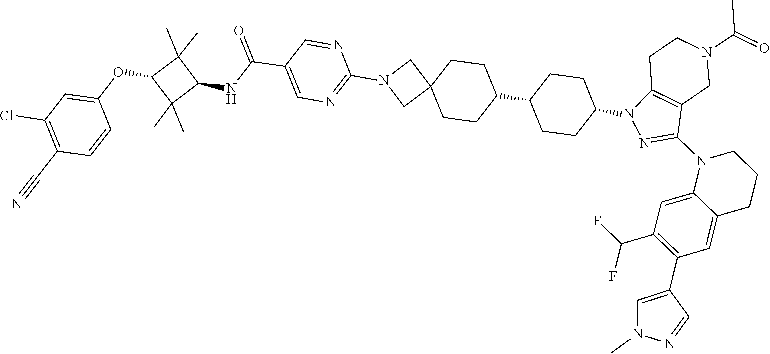 [SMILES: CC(=O)N1CCc2c(c(N3CCCc4cc(-c5cnn(C)c5)c(C(F)F)cc43)nn2[C@H]2CC[C@@H](C3CCC4(CC3)CN(c3ncc(C(=O)N[C@H]5C(C)(C)[C@H](Oc6ccc(C#N)c(Cl)c6)C5(C)C)cn3)C4)CC2)C1] |
| 380 | 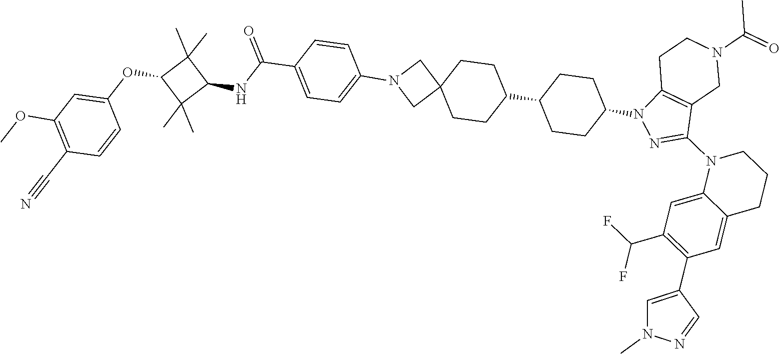 [SMILES: COc1cc(O[C@H]2C(C)(C)[C@H](NC(=O)c3ccc(N4CC5(CCC([C@H]6CC[C@@H](n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 381 | 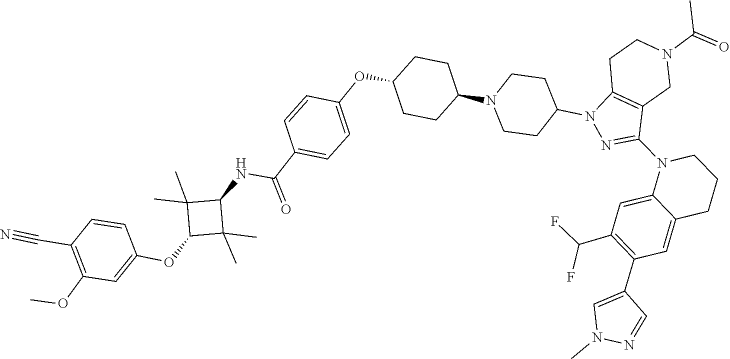 [SMILES: COc1cc(O[C@H]2C(C)(C)[C@H](NC(=O)c3ccc(O[C@H]4CC[C@H](N5CCC(n6nc(N7CCCc8cc(-c9cnn(C)c9)c(C(F)F)cc87)c7c6CCN(C(C)=O)C7)CC5)CC4)cc3)C2(C)C)ccc1C#N] |
| 382 | 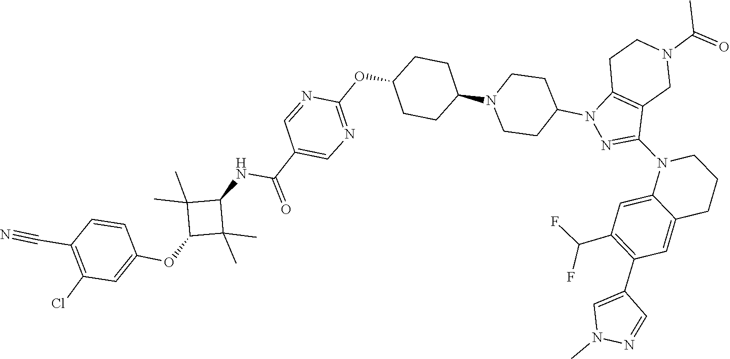 [SMILES: CC(=O)N1CCc2c(c(N3CCCc4cc(-c5cnn(C)c5)c(C(F)F)cc43)nn2C2CCN([C@H]3CC[C@H](Oc4ncc(C(=O)N[C@H]5C(C)(C)[C@H](Oc6ccc(C#N)c(Cl)c6)C5(C)C)cn4)CC3)CC2)C1] |
| 383 | 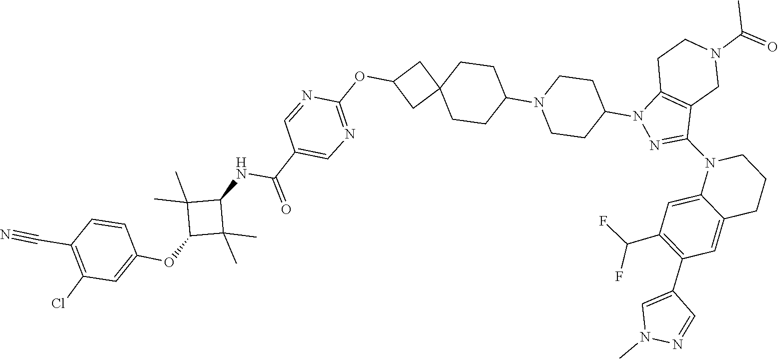 [SMILES: CC(=O)N1CCc2c(c(N3CCCc4cc(-c5cnn(C)c5)c(C(F)F)cc43)nn2C2CCN(C3CCC4(CC3)CC(Oc3ncc(C(=O)N[C@H]5C(C)(C)[C@H](Oc6ccc(C#N)c(Cl)c6)C5(C)C)cn3)C4)CC2)C1] |
| 384 | 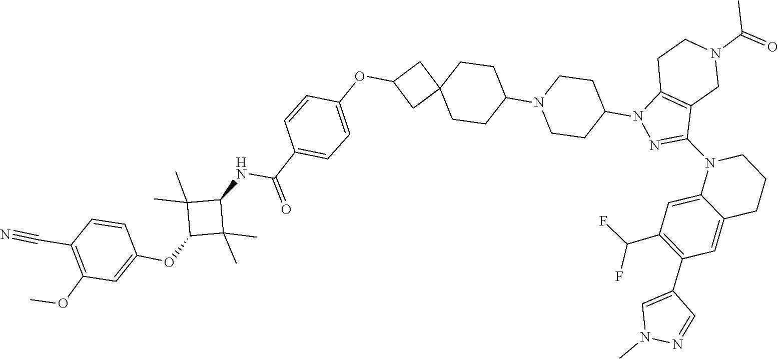 [SMILES: COc1cc(O[C@H]2C(C)(C)[C@H](NC(=O)c3ccc(OC4CC5(CCC(N6CCC(n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 385 | 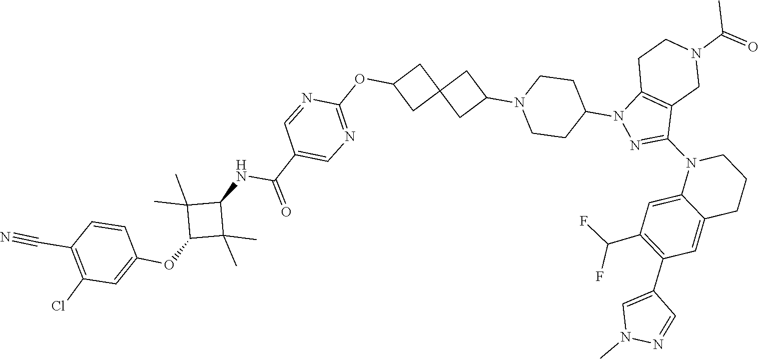 [SMILES: CC(=O)N1CCc2c(c(N3CCCc4cc(-c5cnn(C)c5)c(C(F)F)cc43)nn2C2CCN(C3CC4(CC(Oc5ncc(C(=O)N[C@H]6C(C)(C)[C@H](Oc7ccc(C#N)c(Cl)c7)C6(C)C)cn5)C4)C3)CC2)C1] |
| 386 | 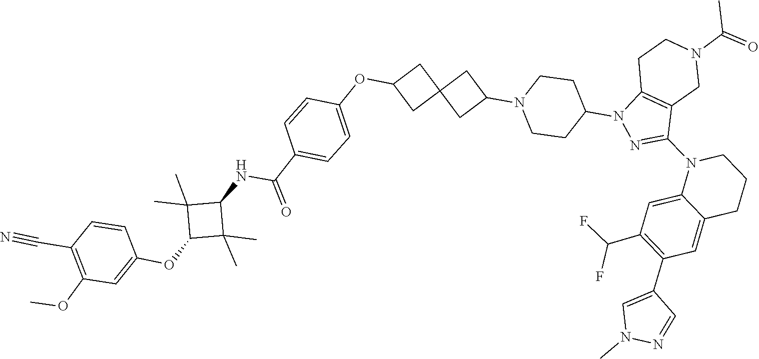 [SMILES: COc1cc(O[C@H]2C(C)(C)[C@H](NC(=O)c3ccc(OC4CC5(C4)CC(N4CCC(n6nc(N7CCCc8cc(-c9cnn(C)c9)c(C(F)F)cc87)c7c6CCN(C(C)=O)C7)CC4)C5)cc3)C2(C)C)ccc1C#N] |
| 387 | 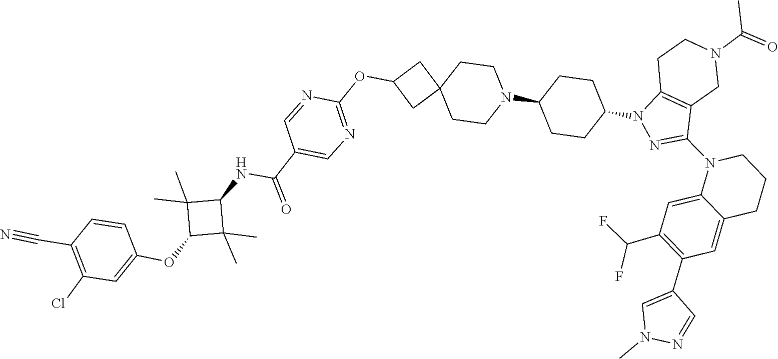 [SMILES: CC(=O)N1CCc2c(c(N3CCCc4cc(-c5cnn(C)c5)c(C(F)F)cc43)nn2[C@H]2CC[C@H](N3CCC4(CC3)CC(Oc3ncc(C(=O)N[C@H]5C(C)(C)[C@H](Oc6ccc(C#N)c(Cl)c6)C5(C)C)cn3)C4)CC2)C1] |
| 388 | 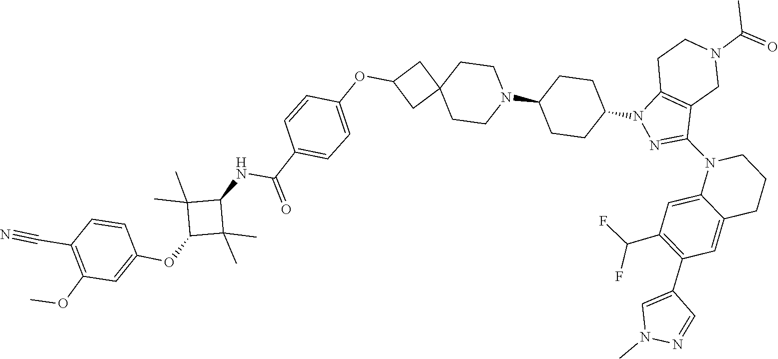 [SMILES: COc1cc(O[C@H]2C(C)(C)[C@H](NC(=O)c3ccc(OC4CC5(CCN([C@H]6CC[C@H](n7nc(N8CCCc9cc(-c%10cnn(C)c%10)c(C(F)F)cc98)c8c7CCN(C(C)=O)C8)CC6)CC5)C4)cc3)C2(C)C)ccc1C#N] |
| 389 | 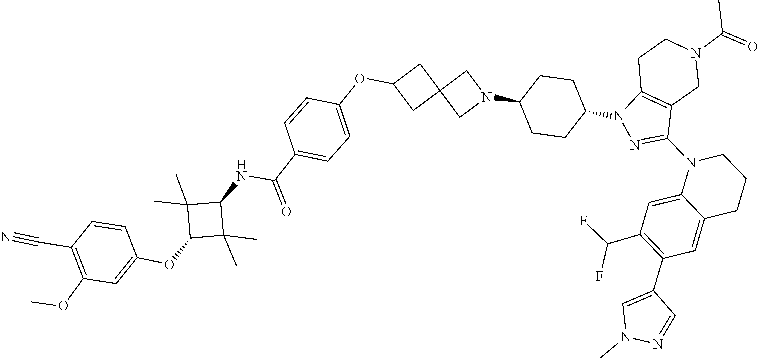 [SMILES: COc1cc(O[C@H]2C(C)(C)[C@H](NC(=O)c3ccc(OC4CC5(C4)CN([C@H]4CC[C@H](n6nc(N7CCCc8cc(-c9cnn(C)c9)c(C(F)F)cc87)c7c6CCN(C(C)=O)C7)CC4)C5)cc3)C2(C)C)ccc1C#N] |
| 390 | 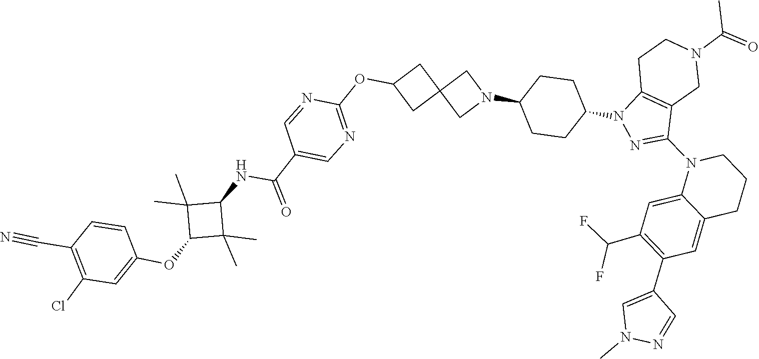 [SMILES: CC(=O)N1CCc2c(c(N3CCCc4cc(-c5cnn(C)c5)c(C(F)F)cc43)nn2[C@H]2CC[C@H](N3CC4(CC(Oc5ncc(C(=O)N[C@H]6C(C)(C)[C@H](Oc7ccc(C#N)c(Cl)c7)C6(C)C)cn5)C4)C3)CC2)C1] |
| 391 | 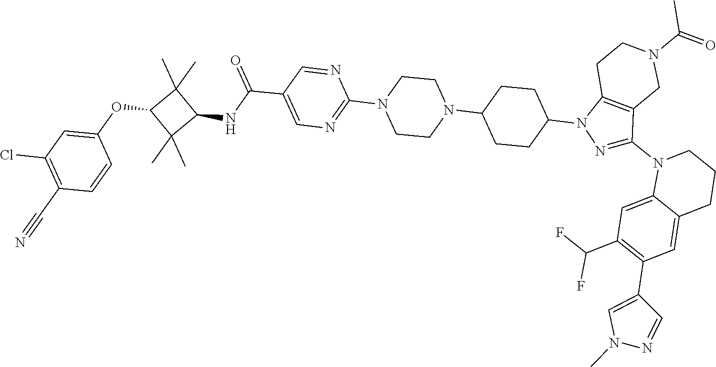 [SMILES: CC(=O)N1CCc2c(c(N3CCCc4cc(-c5cnn(C)c5)c(C(F)F)cc43)nn2C2CCC(N3CCN(c4ncc(C(=O)N[C@H]5C(C)(C)[C@H](Oc6ccc(C#N)c(Cl)c6)C5(C)C)cn4)CC3)CC2)C1] |
| 392 | 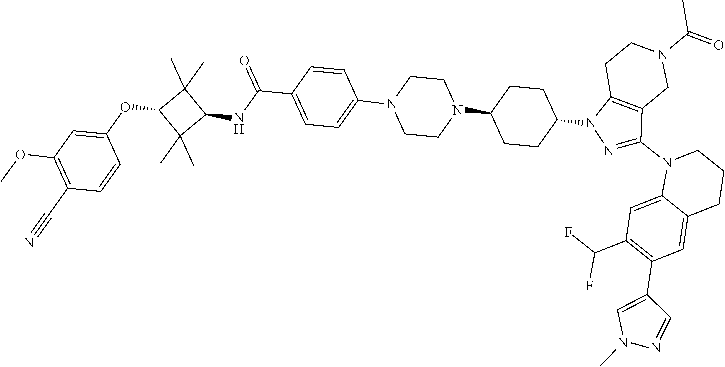 [SMILES: COc1cc(O[C@H]2C(C)(C)[C@H](NC(=O)c3ccc(N4CCN([C@H]5CC[C@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 393 | 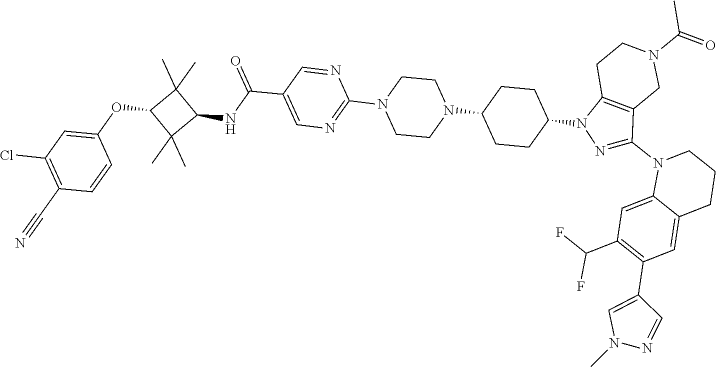 [SMILES: CC(=O)N1CCc2c(c(N3CCCc4cc(-c5cnn(C)c5)c(C(F)F)cc43)nn2[C@H]2CC[C@@H](N3CCN(c4ncc(C(=O)N[C@H]5C(C)(C)[C@H](Oc6ccc(C#N)c(Cl)c6)C5(C)C)cn4)CC3)CC2)C1] |
| 394 | 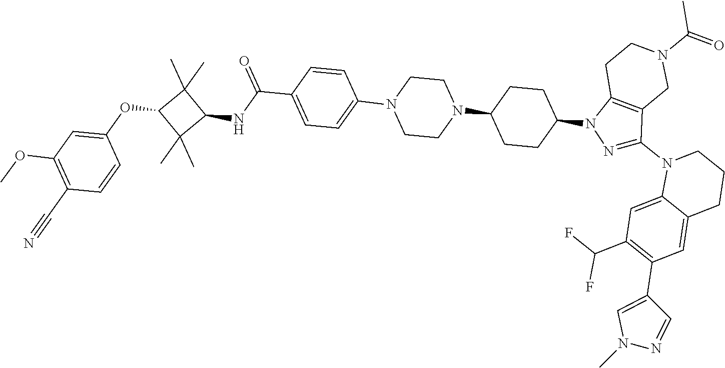 [SMILES: COc1cc(O[C@H]2C(C)(C)[C@H](NC(=O)c3ccc(N4CCN([C@H]5CC[C@@H](n6nc(N7CCCc8cc(-c9cnn(C)c9)c(C(F)F)cc87)c7c6CCN(C(C)=O)C7)CC5)CC4)cc3)C2(C)C)ccc1C#N] |
| 395 | 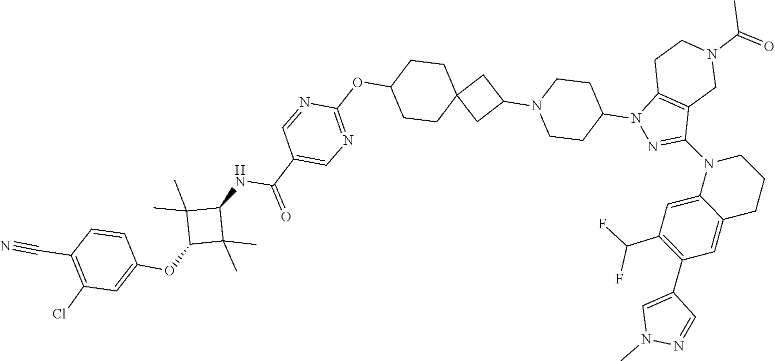 [SMILES: CC(=O)N1CCc2c(c(N3CCCc4cc(-c5cnn(C)c5)c(C(F)F)cc43)nn2C2CCN(C3CC4(CCC(Oc5ncc(C(=O)N[C@H]6C(C)(C)[C@H](Oc7ccc(C#N)c(Cl)c7)C6(C)C)cn5)CC4)C3)CC2)C1] |
| 396 | 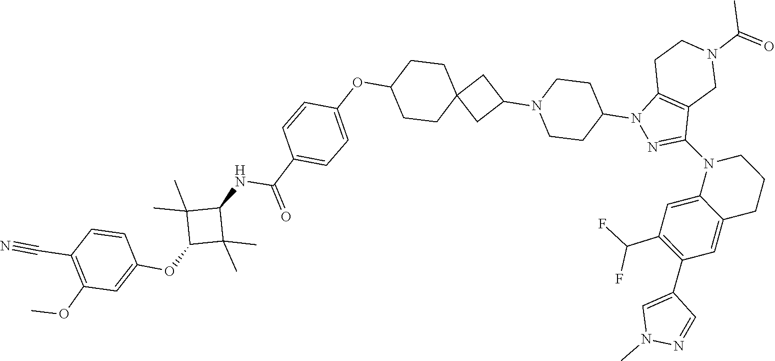 [SMILES: COc1cc(O[C@H]2C(C)(C)[C@H](NC(=O)c3ccc(OC4CCC5(CC4)CC(N4CCC(n6nc(N7CCCc8cc(-c9cnn(C)c9)c(C(F)F)cc87)c7c6CCN(C(C)=O)C7)CC4)C5)cc3)C2(C)C)ccc1C#N] |
| 397 | 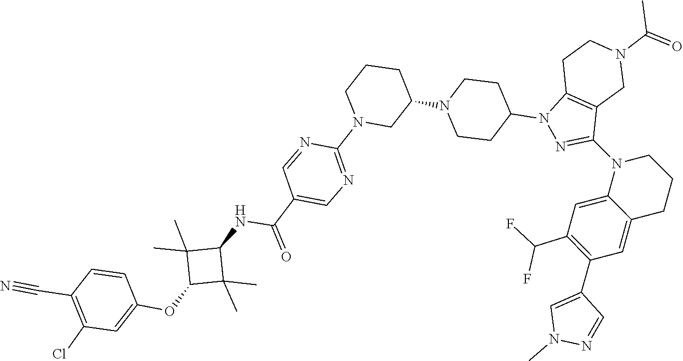 [SMILES: CC(=O)N1CCc2c(c(N3CCCc4cc(-c5cnn(C)c5)c(C(F)F)cc43)nn2C2CCN([C@H]3CCCN(c4ncc(C(=O)N[C@H]5C(C)(C)[C@H](Oc6ccc(C#N)c(Cl)c6)C5(C)C)cn4)C3)CC2)C1] |
| 398 | 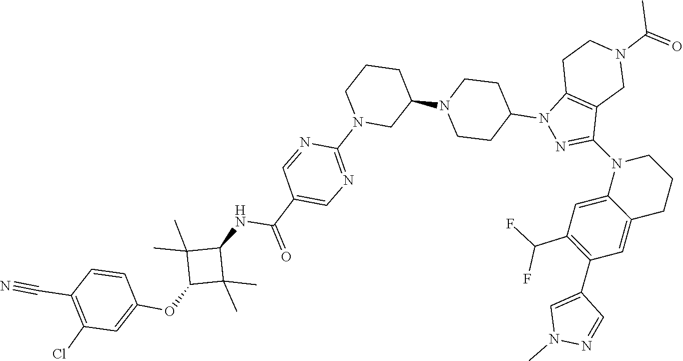 [SMILES: CC(=O)N1CCc2c(c(N3CCCc4cc(-c5cnn(C)c5)c(C(F)F)cc43)nn2C2CCN([C@@H]3CCCN(c4ncc(C(=O)N[C@H]5C(C)(C)[C@H](Oc6ccc(C#N)c(Cl)c6)C5(C)C)cn4)C3)CC2)C1] |
| 399 | 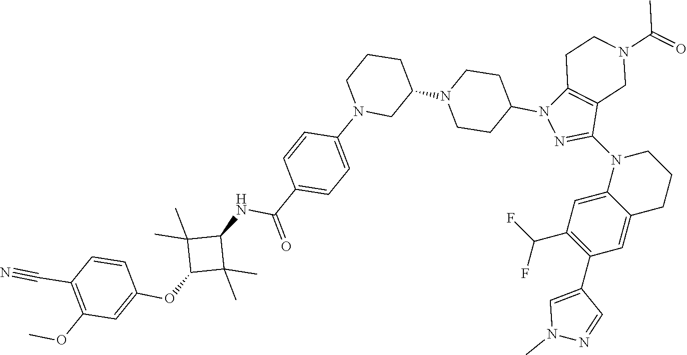 [SMILES: COc1cc(O[C@H]2C(C)(C)[C@H](NC(=O)c3ccc(N4CCC[C@H](N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 400 | 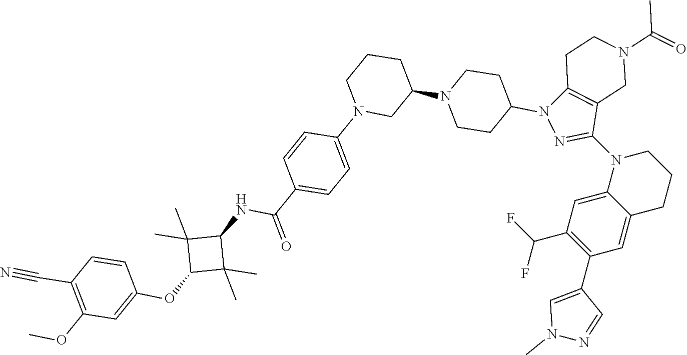 [SMILES: COc1cc(O[C@H]2C(C)(C)[C@H](NC(=O)c3ccc(N4CCC[C@@H](N5CCC(n6nc(N7CCCc8cc(-c9cnn(C)c9)c(C(F)F)cc87)c7c6CCN(C(C)=O)C7)CC5)C4)cc3)C2(C)C)ccc1C#N] |
| 401 | 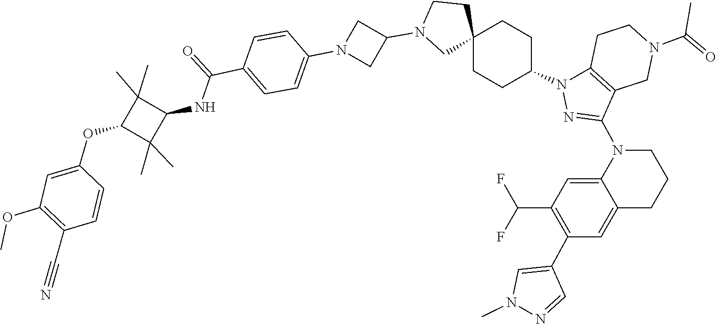 [SMILES: COc1cc(O[C@H]2C(C)(C)[C@H](NC(=O)c3ccc(N4CC(N5CC[C@]6(CC[C@H](n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 402 | 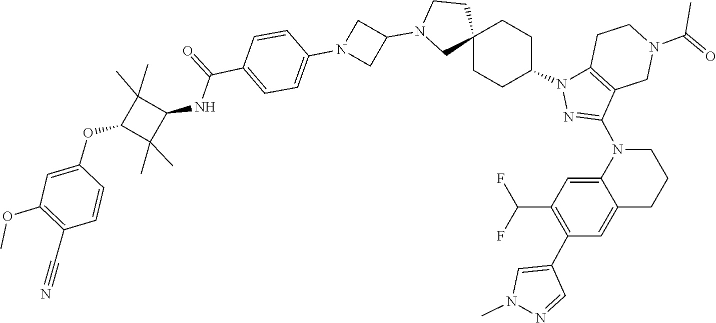 [SMILES: COc1cc(O[C@H]2C(C)(C)[C@H](NC(=O)c3ccc(N4CC(N5CC[C@]6(CC[C@@H](n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 403 | 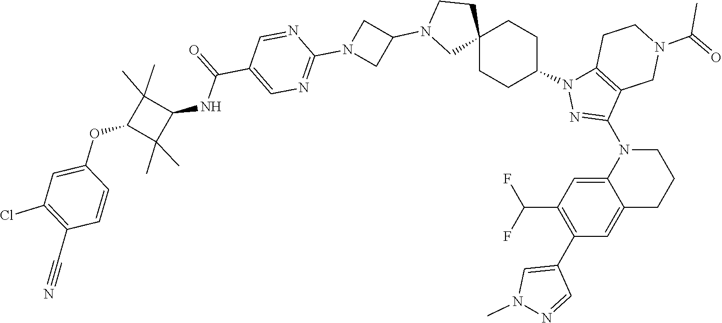 [SMILES: CC(=O)N1CCc2c(c(N3CCCc4cc(-c5cnn(C)c5)c(C(F)F)cc43)nn2[C@H]2CC[C@]3(CCN(C4CN(c5ncc(C(=O)N[C@H]6C(C)(C)[C@H](Oc7ccc(C#N)c(Cl)c7)C6(C)C)cn5)C4)C3)CC2)C1] |
| 404 | 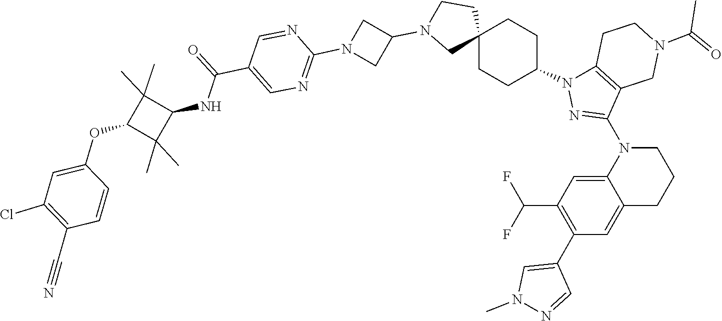 [SMILES: CC(=O)N1CCc2c(c(N3CCCc4cc(-c5cnn(C)c5)c(C(F)F)cc43)nn2[C@H]2CC[C@@]3(CCN(C4CN(c5ncc(C(=O)N[C@H]6C(C)(C)[C@H](Oc7ccc(C#N)c(Cl)c7)C6(C)C)cn5)C4)C3)CC2)C1] |
| 405 | 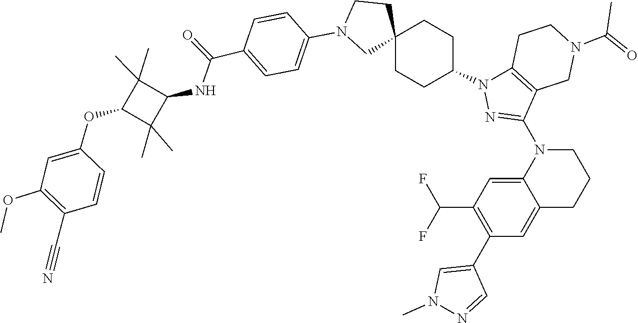 [SMILES: COc1cc(O[C@H]2C(C)(C)[C@H](NC(=O)c3ccc(N4CC[C@]5(CC[C@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 406 | 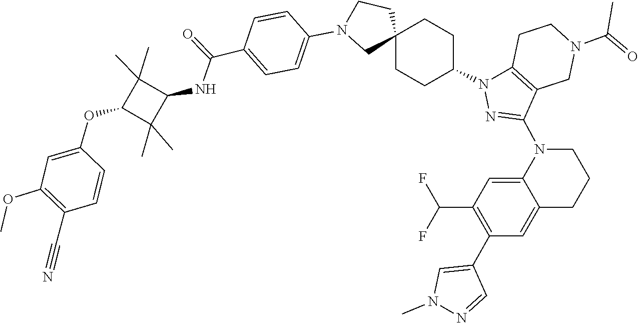 [SMILES: COc1cc(O[C@H]2C(C)(C)[C@H](NC(=O)c3ccc(N4CC[C@]5(CC[C@@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 407 | 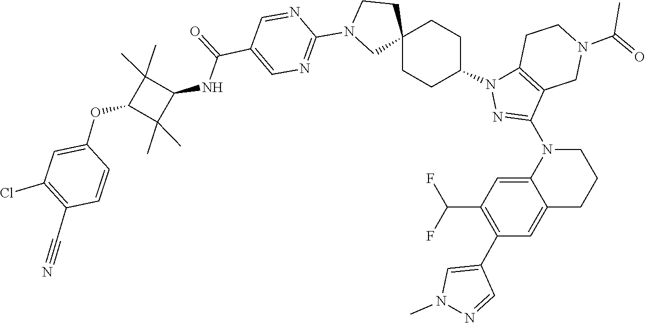 [SMILES: CC(=O)N1CCc2c(c(N3CCCc4cc(-c5cnn(C)c5)c(C(F)F)cc43)nn2[C@H]2CC[C@]3(CCN(c4ncc(C(=O)N[C@H]5C(C)(C)[C@H](Oc6ccc(C#N)c(Cl)c6)C5(C)C)cn4)C3)CC2)C1] |
| 408 | 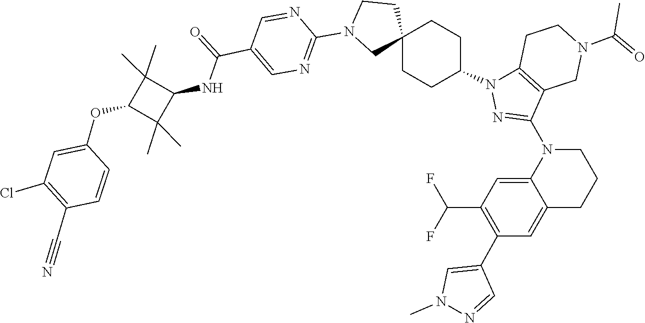 [SMILES: CC(=O)N1CCc2c(c(N3CCCc4cc(-c5cnn(C)c5)c(C(F)F)cc43)nn2[C@H]2CC[C@@]3(CCN(c4ncc(C(=O)N[C@H]5C(C)(C)[C@H](Oc6ccc(C#N)c(Cl)c6)C5(C)C)cn4)C3)CC2)C1] |
| 409 | 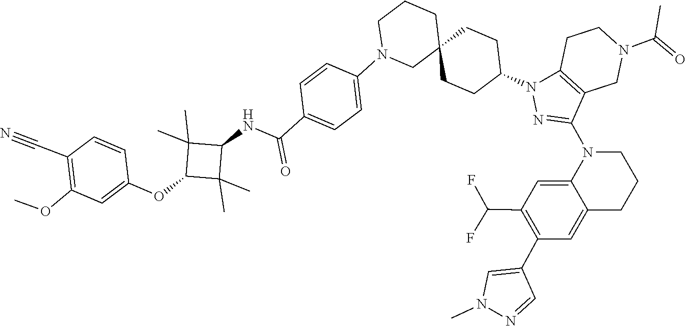 [SMILES: COc1cc(O[C@H]2C(C)(C)[C@H](NC(=O)c3ccc(N4CCC[C@]5(CC[C@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 410 | 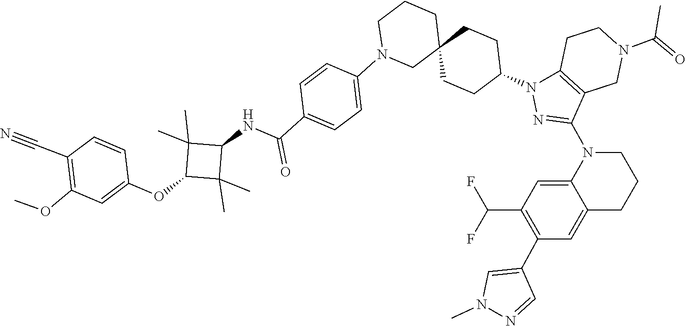 [SMILES: COc1cc(O[C@H]2C(C)(C)[C@H](NC(=O)c3ccc(N4CCC[C@]5(CC[C@@H](n6nc(N7CCCc8cc(-c9cnn(C)c9)c(C(F)F)cc87)c7c6CCN(C(C)=O)C7)CC5)C4)cc3)C2(C)C)ccc1C#N] |
| 411 | 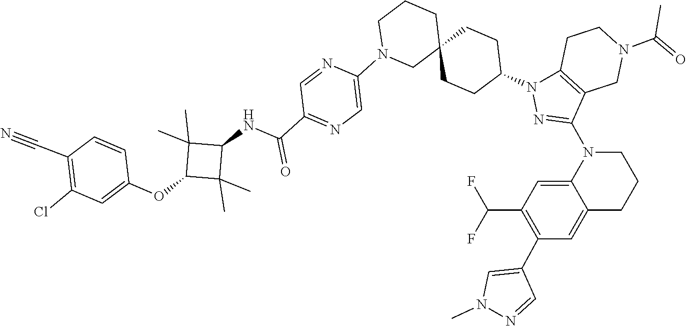 [SMILES: CC(=O)N1CCc2c(c(N3CCCc4cc(-c5cnn(C)c5)c(C(F)F)cc43)nn2[C@H]2CC[C@]3(CCCN(c4cnc(C(=O)N[C@H]5C(C)(C)[C@H](Oc6ccc(C#N)c(Cl)c6)C5(C)C)cn4)C3)CC2)C1] |
| 412 | 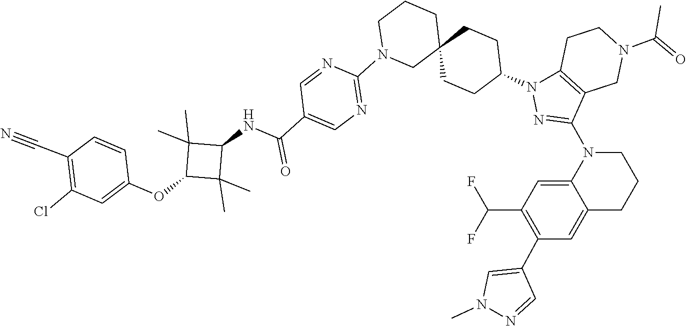 [SMILES: CC(=O)N1CCc2c(c(N3CCCc4cc(-c5cnn(C)c5)c(C(F)F)cc43)nn2[C@H]2CC[C@@]3(CCCN(c4ncc(C(=O)N[C@H]5C(C)(C)[C@H](Oc6ccc(C#N)c(Cl)c6)C5(C)C)cn4)C3)CC2)C1] |
| 413 | 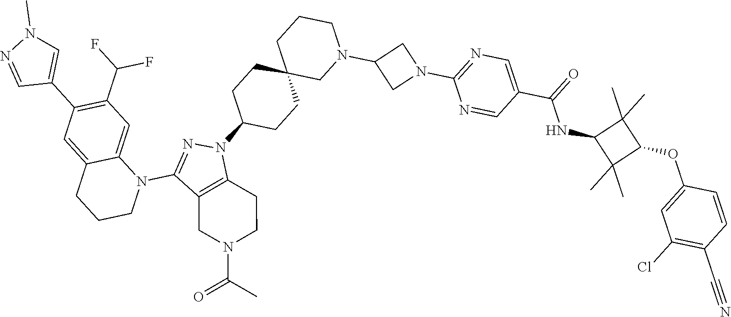 [SMILES: CC(=O)N1CCc2c(c(N3CCCc4cc(-c5cnn(C)c5)c(C(F)F)cc43)nn2[C@H]2CC[C@]3(CCCN(C4CN(c5ncc(C(=O)N[C@H]6C(C)(C)[C@H](Oc7ccc(C#N)c(Cl)c7)C6(C)C)cn5)C4)C3)CC2)C1] |
| 414 | 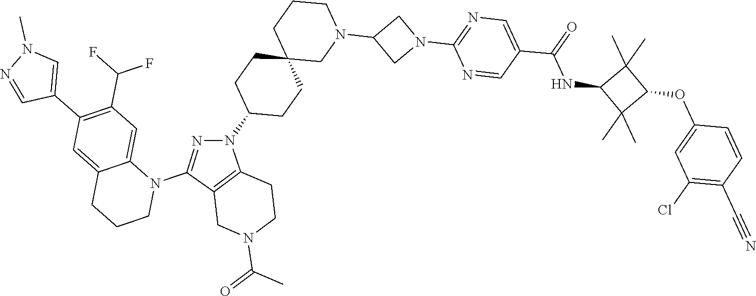 [SMILES: CC(=O)N1CCc2c(c(N3CCCc4cc(-c5cnn(C)c5)c(C(F)F)cc43)nn2[C@H]2CC[C@@]3(CCCN(C4CN(c5ncc(C(=O)N[C@H]6C(C)(C)[C@H](Oc7ccc(C#N)c(Cl)c7)C6(C)C)cn5)C4)C3)CC2)C1] |
| 415 | 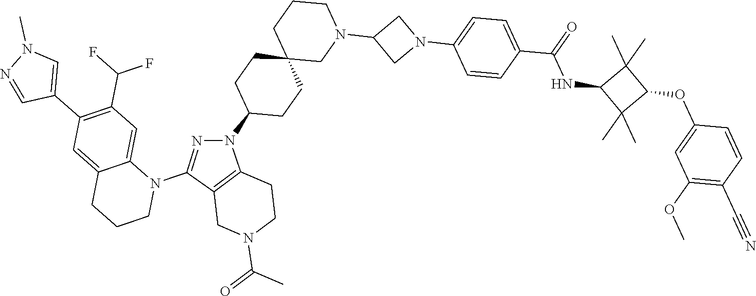 [SMILES: COc1cc(O[C@H]2C(C)(C)[C@H](NC(=O)c3ccc(N4CC(N5CCC[C@]6(CC[C@H](n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 416 | 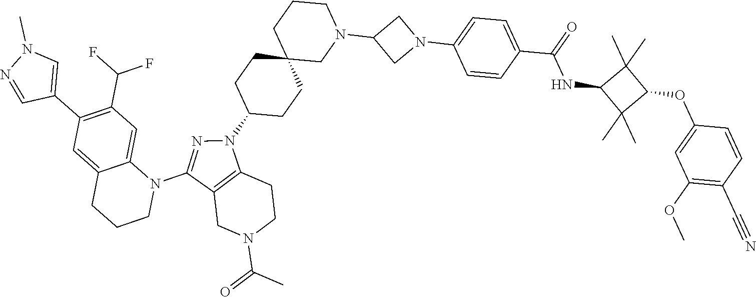 [SMILES: COc1cc(O[C@H]2C(C)(C)[C@H](NC(=O)c3ccc(N4CC(N5CCC[C@]6(CC[C@@H](n7nc(N8CCCc9cc(-c%10cnn(C)c%10)c(C(F)F)cc98)c8c7CCN(C(C)=O)C8)CC6)C5)C4)cc3)C2(C)C)ccc1C#N] |
| 417 | 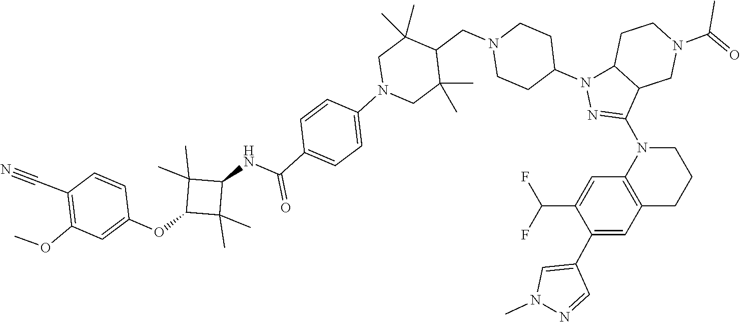 [SMILES: COc1cc(O[C@H]2C(C)(C)[C@H](NC(=O)c3ccc(N4CC(C)(C)C(CN5CCC(N6N=C(N7CCCc8cc(-c9cnn(C)c9)c(C(F)F)cc87)C7CN(C(C)=O)CCC76)CC5)C(C)(C)C4)cc3)C2(C)C)ccc1C#N] |
| 418 | 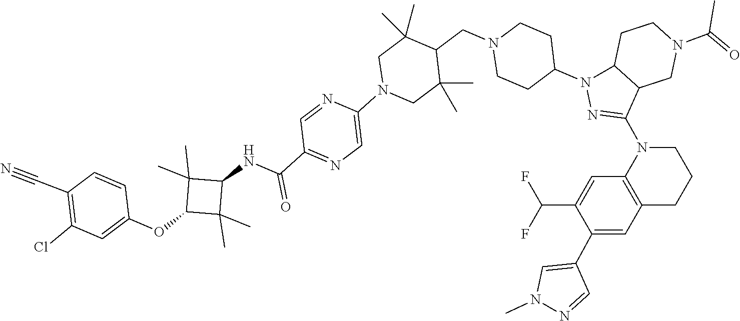 [SMILES: CC(=O)N1CCC2C(C1)C(N1CCCc3cc(-c4cnn(C)c4)c(C(F)F)cc31)=NN2C1CCN(CC2C(C)(C)CN(c3cnc(C(=O)N[C@H]4C(C)(C)[C@H](Oc5ccc(C#N)c(Cl)c5)C4(C)C)cn3)CC2(C)C)CC1] |
| 419 | 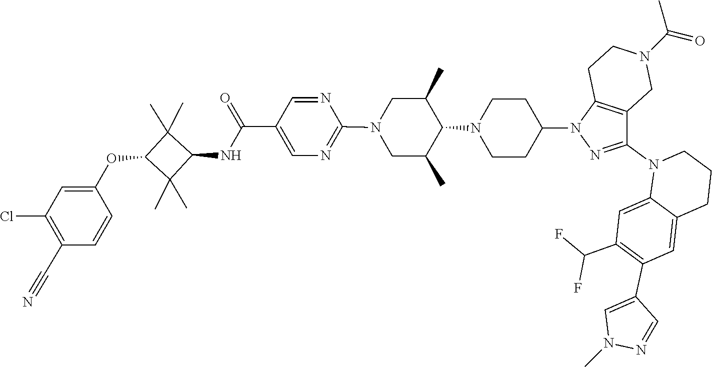 [SMILES: CC(=O)N1CCc2c(c(N3CCCc4cc(-c5cnn(C)c5)c(C(F)F)cc43)nn2C2CCN([C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 420 | 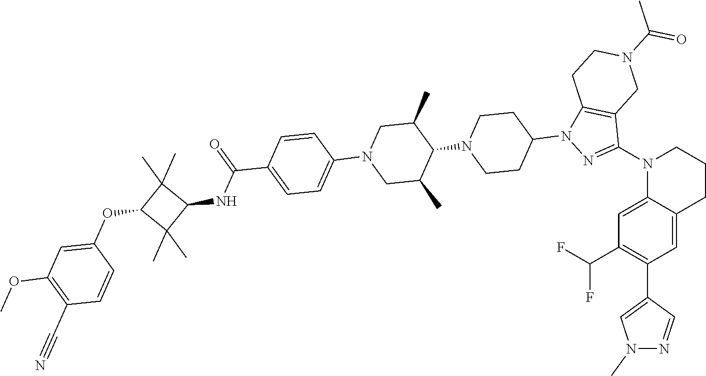 [SMILES: COc1cc(O[C@H]2C(C)(C)[C@H](NC(=O)c3ccc(N4C[C@@H](C)[C@H](N5CCC(n6nc(N7CCCc8cc(-c9cnn(C)c9)c(C(F)F)cc87)c7c6CCN(C(C)=O)C7)CC5)[C@@H](C)C4)cc3)C2(C)C)ccc1C#N] |
| 421 | 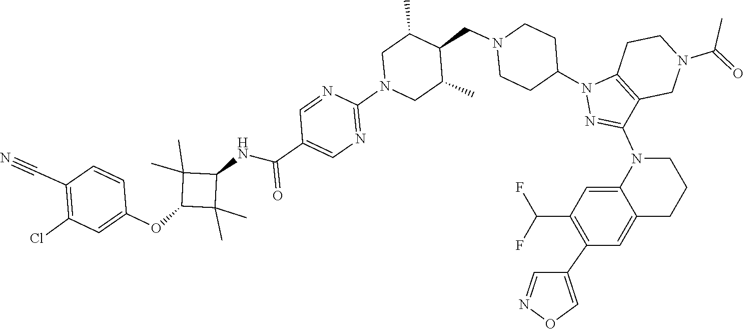 [SMILES: CC(=O)N1CCc2c(c(N3CCCc4cc(-c5cnoc5)c(C(F)F)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 422 | 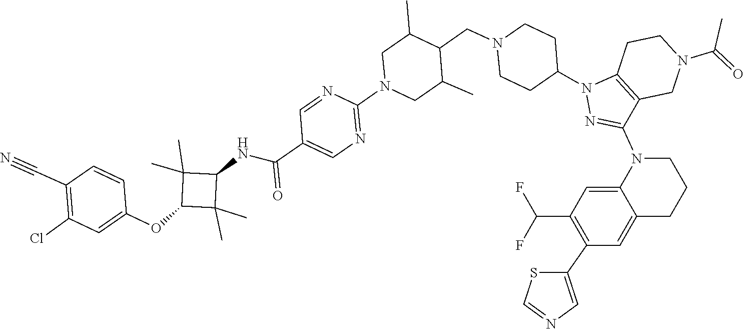 [SMILES: CC(=O)N1CCc2c(c(N3CCCc4cc(-c5cncs5)c(C(F)F)cc43)nn2C2CCN(CC3C(C)CN(c4ncc(C(=O)N[C@H]5C(C)(C)[C@H](Oc6ccc(C#N)c(Cl)c6)C5(C)C)cn4)CC3C)CC2)C1] |
| 423 | 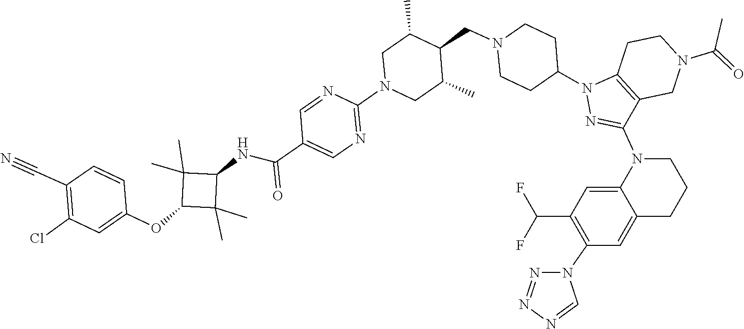 [SMILES: CC(=O)N1CCc2c(c(N3CCCc4cc(-n5cnnn5)c(C(F)F)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 424 | 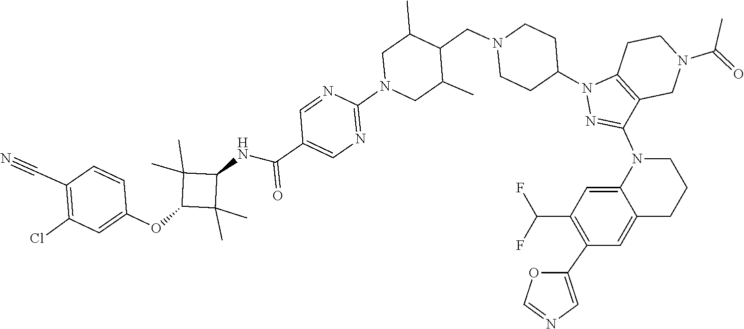 [SMILES: CC(=O)N1CCc2c(c(N3CCCc4cc(-c5cnco5)c(C(F)F)cc43)nn2C2CCN(CC3C(C)CN(c4ncc(C(=O)N[C@H]5C(C)(C)[C@H](Oc6ccc(C#N)c(Cl)c6)C5(C)C)cn4)CC3C)CC2)C1] |
| 425 | 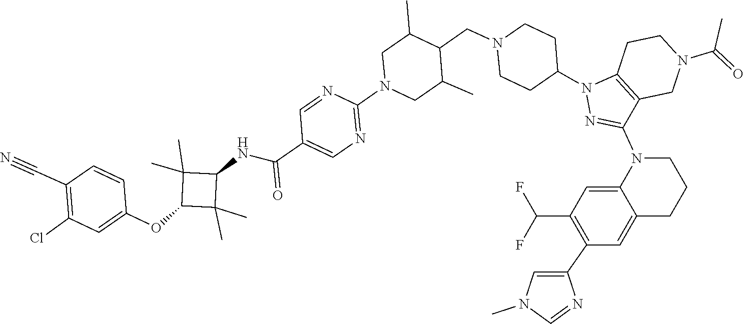 [SMILES: CC(=O)N1CCc2c(c(N3CCCc4cc(-c5cn(C)cn5)c(C(F)F)cc43)nn2C2CCN(CC3C(C)CN(c4ncc(C(=O)N[C@H]5C(C)(C)[C@H](Oc6ccc(C#N)c(Cl)c6)C5(C)C)cn4)CC3C)CC2)C1] |
| 426 | 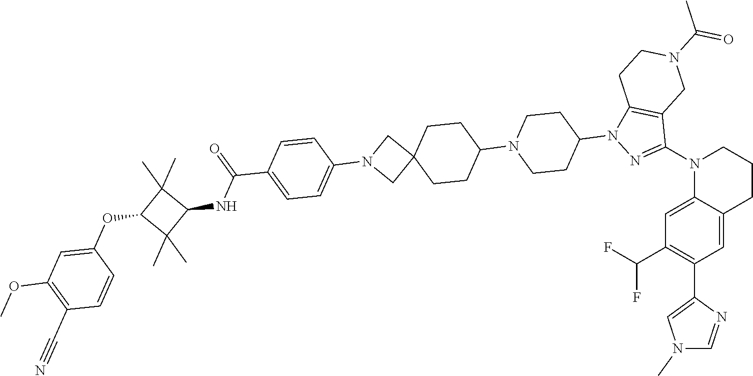 [SMILES: COc1cc(O[C@H]2C(C)(C)[C@H](NC(=O)c3ccc(N4CC5(CCC(N6CCC(n7nc(N8CCCc9cc(-c%10cn(C)cn%10)c(C(F)F)cc98)c8c7CCN(C(C)=O)C8)CC6)CC5)C4)cc3)C2(C)C)ccc1C#N] |
| 427 | 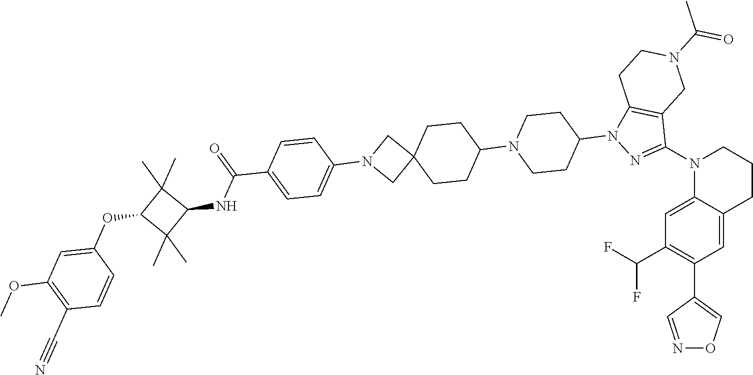 [SMILES: COc1cc(O[C@H]2C(C)(C)[C@H](NC(=O)c3ccc(N4CC5(CCC(N6CCC(n7nc(N8CCCc9cc(-c%10cnoc%10)c(C(F)F)cc98)c8c7CCN(C(C)=O)C8)CC6)CC5)C4)cc3)C2(C)C)ccc1C#N] |
| 428 | 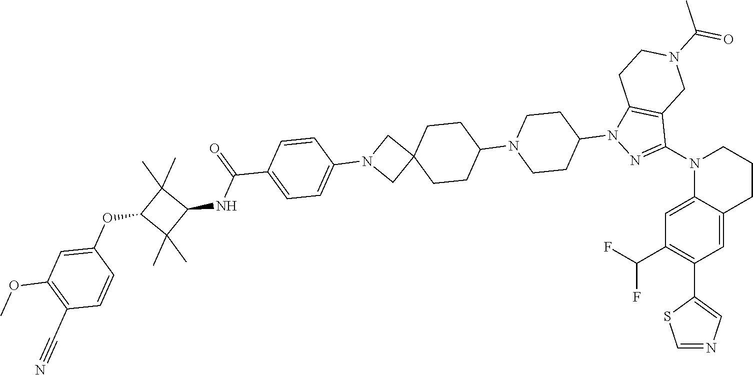 [SMILES: COc1cc(O[C@H]2C(C)(C)[C@H](NC(=O)c3ccc(N4CC5(CCC(N6CCC(n7nc(N8CCCc9cc(-c%10cncs%10)c(C(F)F)cc98)c8c7CCN(C(C)=O)C8)CC6)CC5)C4)cc3)C2(C)C)ccc1C#N] |
| 429 | 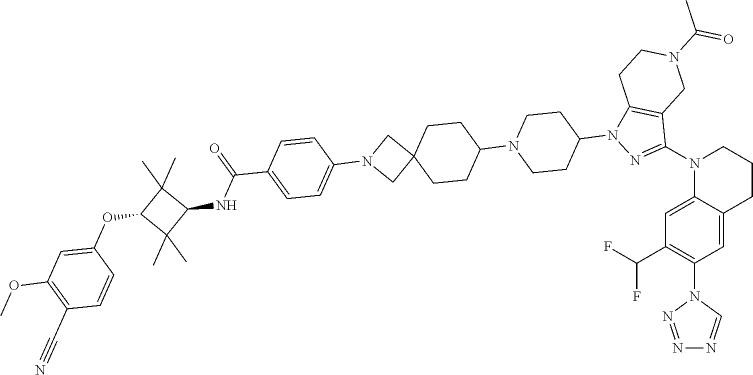 [SMILES: COc1cc(O[C@H]2C(C)(C)[C@H](NC(=O)c3ccc(N4CC5(CCC(N6CCC(n7nc(N8CCCc9cc(-n%10cnnn%10)c(C(F)F)cc98)c8c7CCN(C(C)=O)C8)CC6)CC5)C4)cc3)C2(C)C)ccc1C#N] |
| 430 | 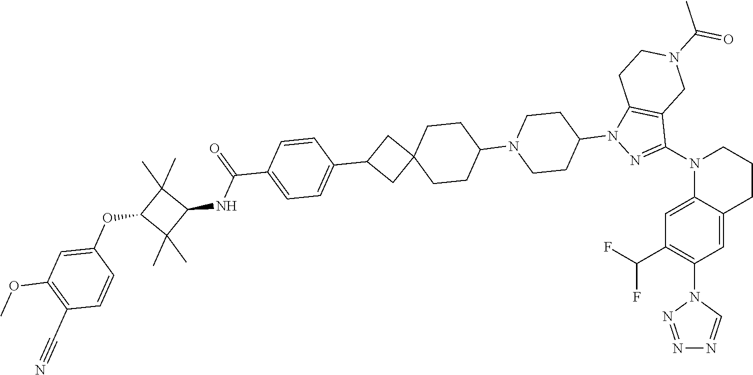 [SMILES: COc1cc(O[C@H]2C(C)(C)[C@H](NC(=O)c3ccc(C4CC5(CCC(N6CCC(n7nc(N8CCCc9cc(-n%10cnnn%10)c(C(F)F)cc98)c8c7CCN(C(C)=O)C8)CC6)CC5)C4)cc3)C2(C)C)ccc1C#N] |
| 431 | 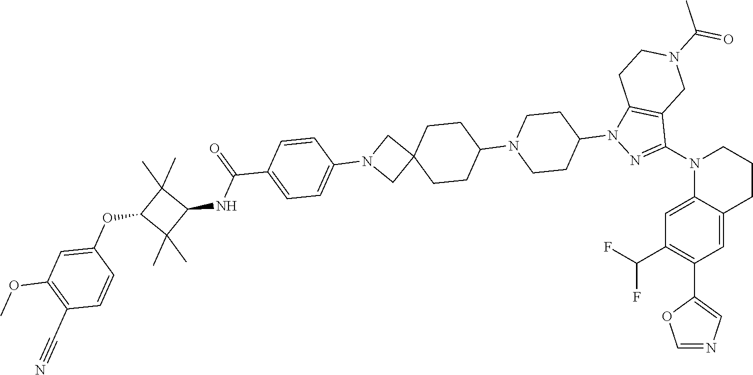 [SMILES: COc1cc(O[C@H]2C(C)(C)[C@H](NC(=O)c3ccc(N4CC5(CCC(N6CCC(n7nc(N8CCCc9cc(-c%10cnco%10)c(C(F)F)cc98)c8c7CCN(C(C)=O)C8)CC6)CC5)C4)cc3)C2(C)C)ccc1C#N] |
| 432 | 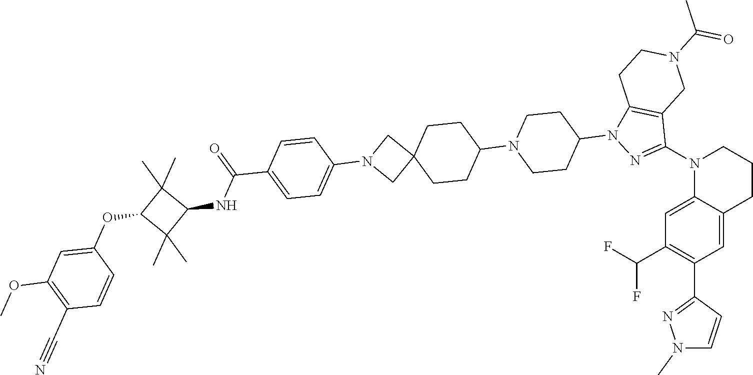 [SMILES: COc1cc(O[C@H]2C(C)(C)[C@H](NC(=O)c3ccc(N4CC5(CCC(N6CCC(n7nc(N8CCCc9cc(-c%10ccn(C)n%10)c(C(F)F)cc98)c8c7CCN(C(C)=O)C8)CC6)CC5)C4)cc3)C2(C)C)ccc1C#N] |
| 433 | 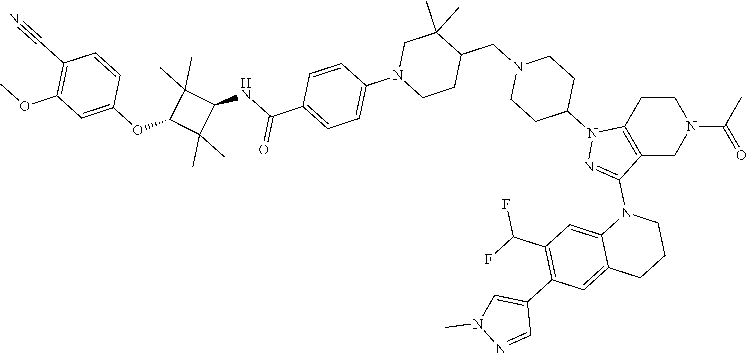 [SMILES: COc1cc(O[C@H]2C(C)(C)[C@H](NC(=O)c3ccc(N4CCC(CN5CCC(n6nc(N7CCCc8cc(-c9cnn(C)c9)c(C(F)F)cc87)c7c6CCN(C(C)=O)C7)CC5)C(C)(C)C4)cc3)C2(C)C)ccc1C#N] |
| 434 | 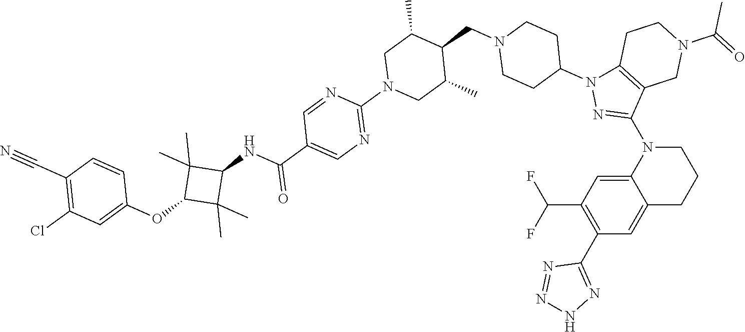 [SMILES: CC(=O)N1CCc2c(c(N3CCCc4cc(-c5nn[nH]n5)c(C(F)F)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 435 | 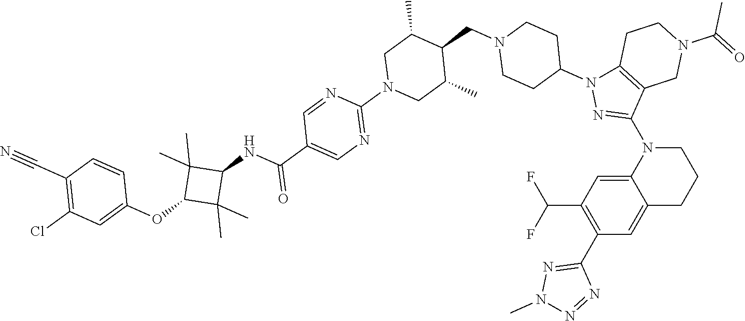 [SMILES: CC(=O)N1CCc2c(c(N3CCCc4cc(-c5nnn(C)n5)c(C(F)F)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 436 | 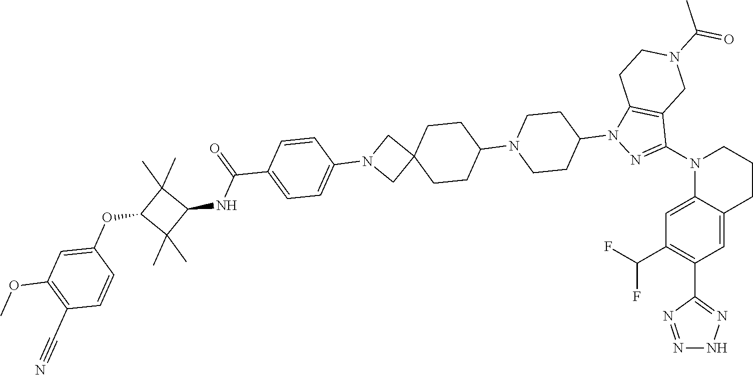 [SMILES: COc1cc(O[C@H]2C(C)(C)[C@H](NC(=O)c3ccc(N4CC5(CCC(N6CCC(n7nc(N8CCCc9cc(-c%10nn[nH]n%10)c(C(F)F)cc98)c8c7CCN(C(C)=O)C8)CC6)CC5)C4)cc3)C2(C)C)ccc1C#N] |
| 437 | 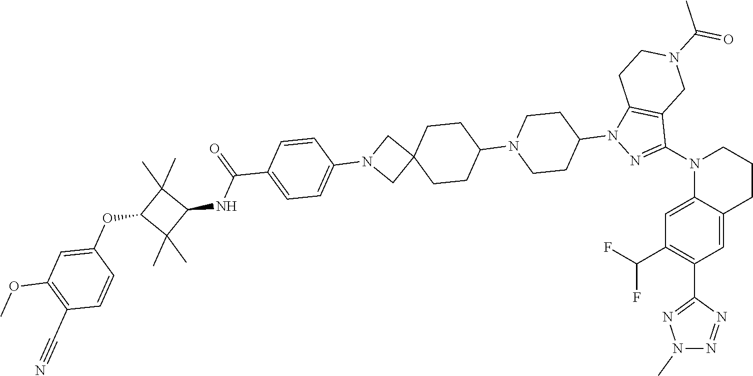 [SMILES: COc1cc(O[C@H]2C(C)(C)[C@H](NC(=O)c3ccc(N4CC5(CCC(N6CCC(n7nc(N8CCCc9cc(-c%10nnn(C)n%10)c(C(F)F)cc98)c8c7CCN(C(C)=O)C8)CC6)CC5)C4)cc3)C2(C)C)ccc1C#N] |
| 438 | 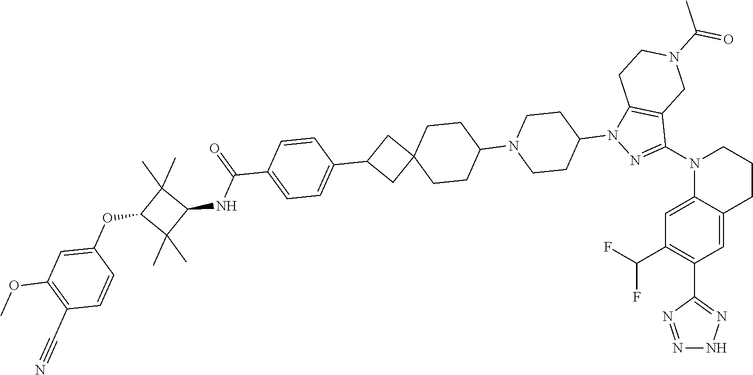 [SMILES: COc1cc(O[C@H]2C(C)(C)[C@H](NC(=O)c3ccc(C4CC5(CCC(N6CCC(n7nc(N8CCCc9cc(-c%10nn[nH]n%10)c(C(F)F)cc98)c8c7CCN(C(C)=O)C8)CC6)CC5)C4)cc3)C2(C)C)ccc1C#N] |
| 439 | 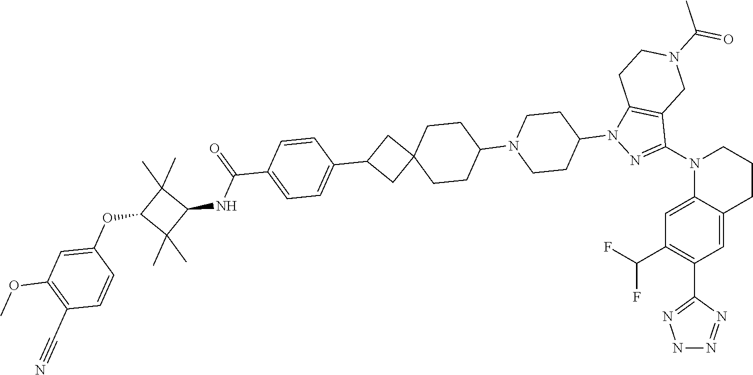 [SMILES: COc1cc(O[C@H]2C(C)(C)[C@H](NC(=O)c3ccc(C4CC5(CCC(N6CCC(n7nc(N8CCCc9cc(-c%10nn[nH]n%10)c(C(F)F)cc98)c8c7CCN(C(C)=O)C8)CC6)CC5)C4)cc3)C2(C)C)ccc1C#N] |
| 440 | 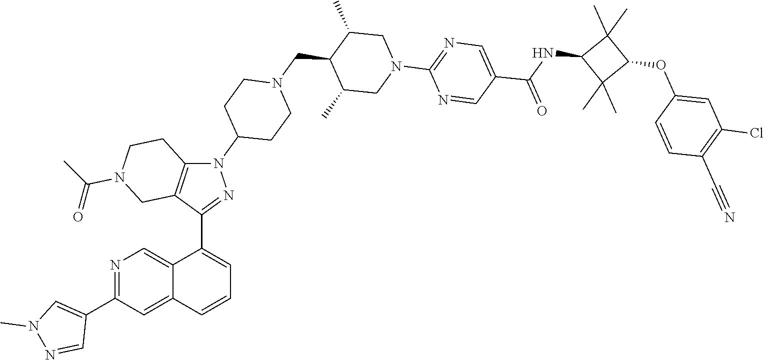 [SMILES: CC(=O)N1CCc2c(c(-c3cccc4cc(-c5cnn(C)c5)ncc34)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 441 | 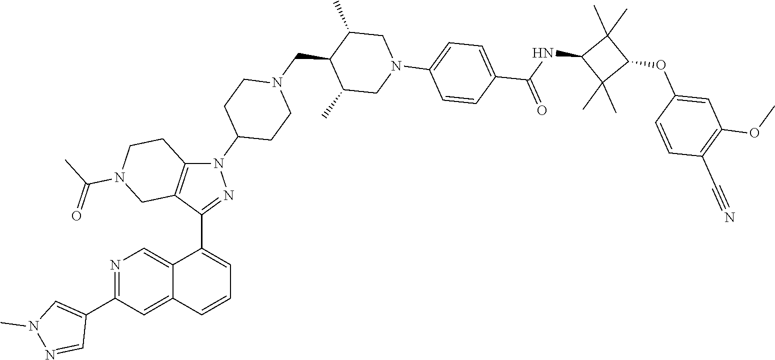 |
| 442 | 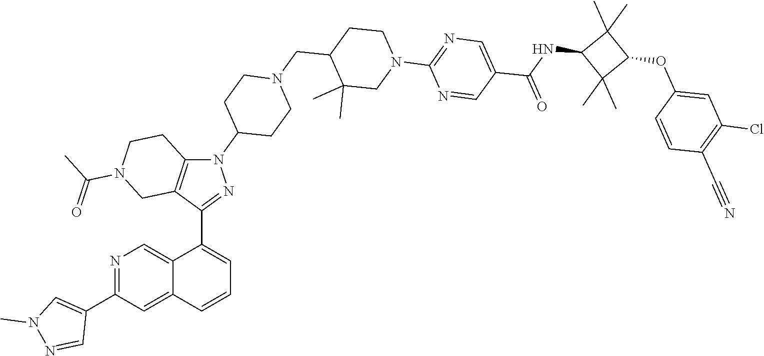 [SMILES: CC(=O)N1CCc2c(c(-c3cccc4cc(-c5cnn(C)c5)ncc34)nn2C2CCN(CC3CCN(c4ncc(C(=O)N[C@H]5C(C)(C)[C@H](Oc6ccc(C#N)c(Cl)c6)C5(C)C)cn4)CC3(C)C)CC2)C1] |
| 443 | 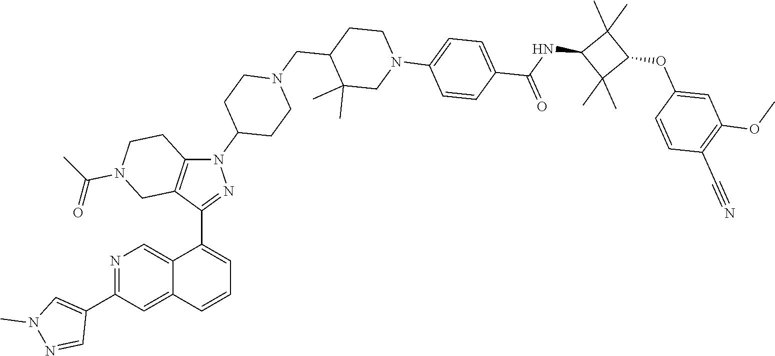 |
| 444 | 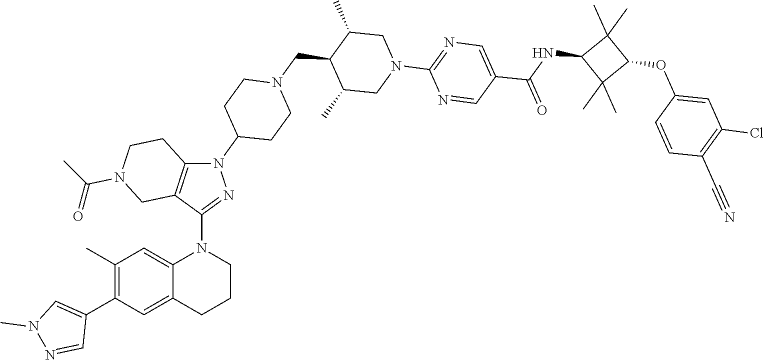 [SMILES: CC(=O)N1CCc2c(c(N3CCCc4cc(-c5cnn(C)c5)c(C)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 445 | 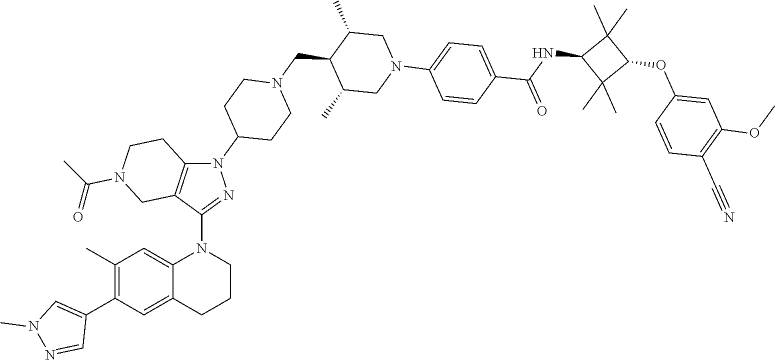 |
| 446 | 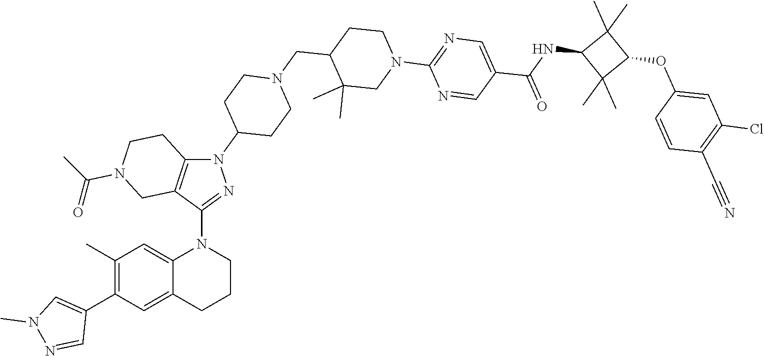 [SMILES: CC(=O)N1CCc2c(c(N3CCCc4cc(-c5cnn(C)c5)c(C)cc43)nn2C2CCN(CC3CCN(c4ncc(C(=O)N[C@H]5C(C)(C)[C@H](Oc6ccc(C#N)c(Cl)c6)C5(C)C)cn4)CC3(C)C)CC2)C1] |
| 447 | 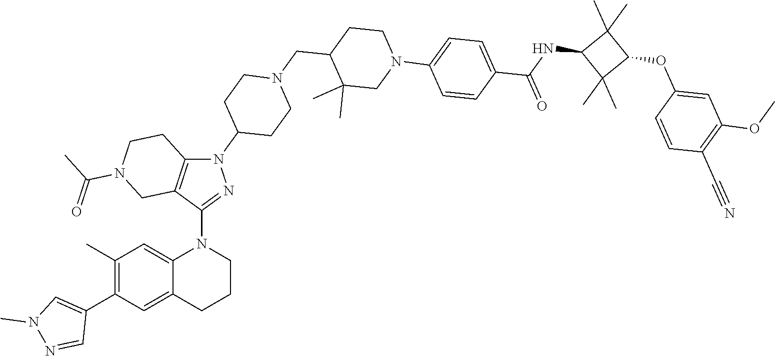 |
| 448 | 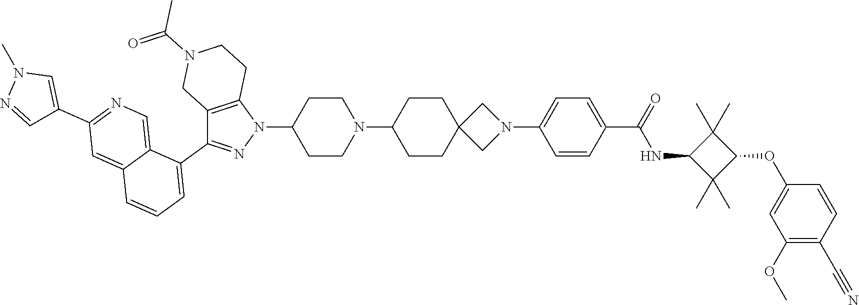 [SMILES: COc1cc(O[C@H]2C(C)(C)[C@H](NC(=O)c3ccc(N4CC5(CCC(N6CCC(n7nc(-c8cccc9cc(-c%10cnn(C)c%10)ncc89)c8c7CCN(C(C)=O)C8)CC6)CC5)C4)cc3)C2(C)C)ccc1C#N] |
| 449 | 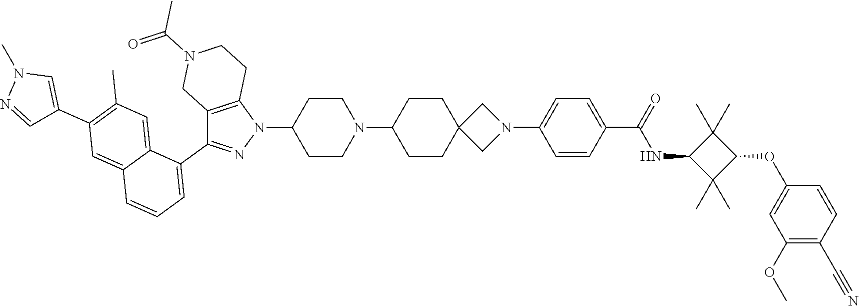 [SMILES: COc1cc(O[C@H]2C(C)(C)[C@H](NC(=O)c3ccc(N4CC5(CCC(N6CCC(n7nc(-c8cccc9cc(-c%10cnn(C)c%10)c(C)cc89)c8c7CCN(C(C)=O)C8)CC6)CC5)C4)cc3)C2(C)C)ccc1C#N] |
| 450 | 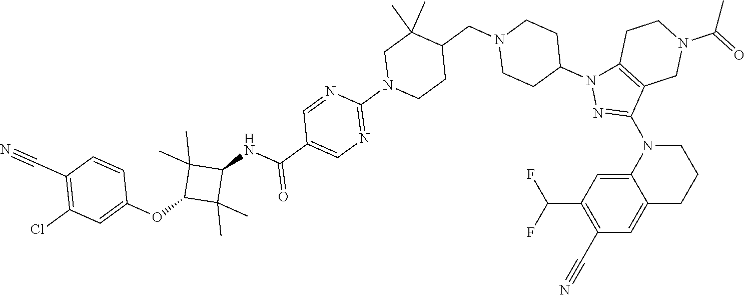 [SMILES: CC(=O)N1CCc2c(c(N3CCCc4cc(C#N)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3(C)C)CC2)C1] |
| 451 | 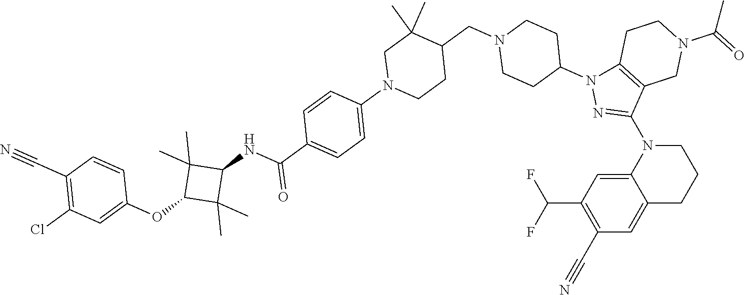 [SMILES: CC(=O)N1CCc2c(c(N3CCCc4cc(C#N)c(C(F)F)cc43)nn2C2CCN(CC3CCN(c4ccc(C(=O)N[C@H]5C(C)(C)[C@H](Oc6ccc(C#N)c(Cl)c6)C5(C)C)cc4)CC3(C)C)CC2)C1] |
| 452 | 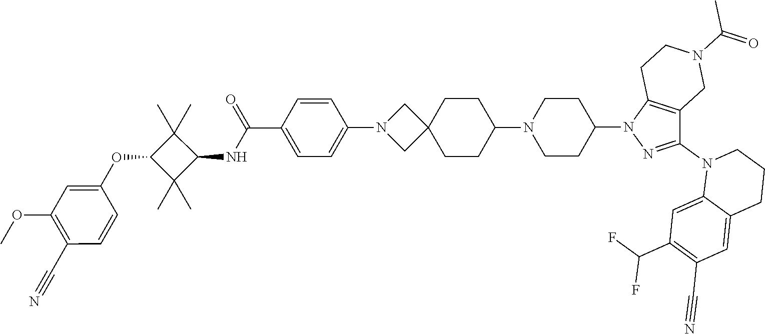 [SMILES: COc1cc(O[C@H]2C(C)(C)[C@H](NC(=O)c3ccc(N4CC5(CCC(N6CCC(n7nc(N8CCCc9cc(C#N)c(C(F)F)cc98)c8c7CCN(C(C)=O)C8)CC6)CC5)C4)cc3)C2(C)C)ccc1C#N] |
| 453 | 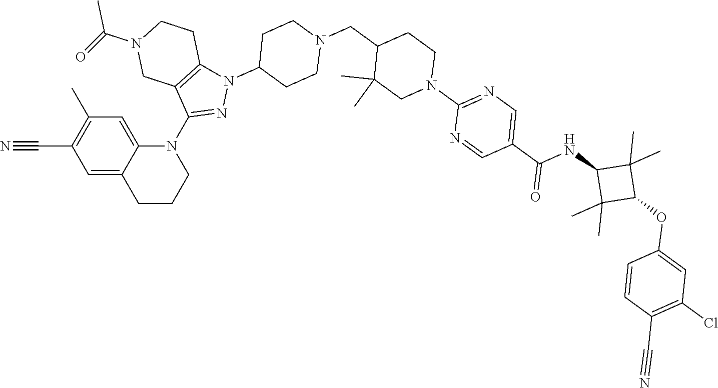 [SMILES: CC(=O)N1CCc2c(c(N3CCCc4cc(C#N)c(C)cc43)nn2C2CCN(CC3CCN(c4ncc(C(=O)N[C@H]5C(C)(C)[C@H](Oc6ccc(C#N)c(Cl)c6)C5(C)C)cn4)CC3(C)C)CC2)C1] |
| 454 | 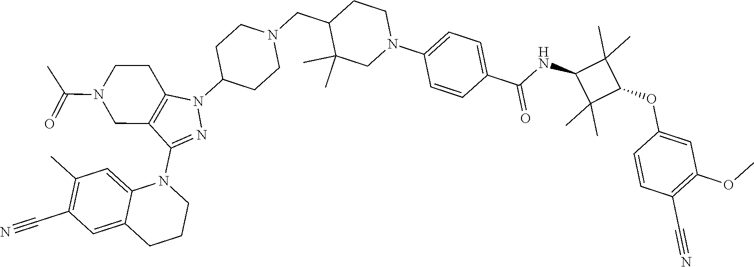 [SMILES: COc1cc(O[C@H]2C(C)(C)[C@H](NC(=O)c3ccc(N4CCC(CN5CCC(n6nc(N7CCCc8cc(C#N)c(C)cc87)c7c6CCN(C(C)=O)C7)CC5)C(C)(C)C4)cc3)C2(C)C)ccc1C#N] |
| 455 | 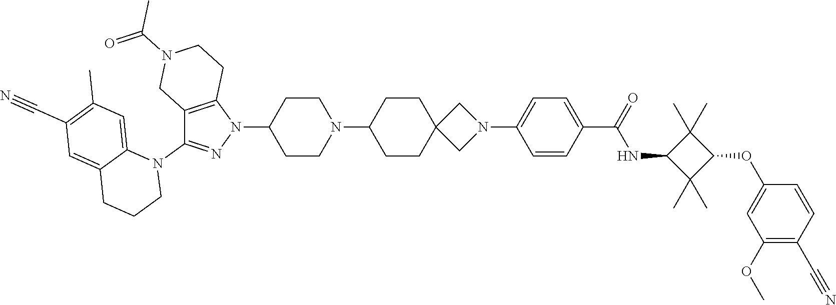 [SMILES: COc1cc(O[C@H]2C(C)(C)[C@H](NC(=O)c3ccc(N4CC5(CCC(N6CCC(n7nc(N8CCCc9cc(C#N)c(C)cc98)c8c7CCN(C(C)=O)C8)CC6)CC5)C4)cc3)C2(C)C)ccc1C#N] |
| 456 | 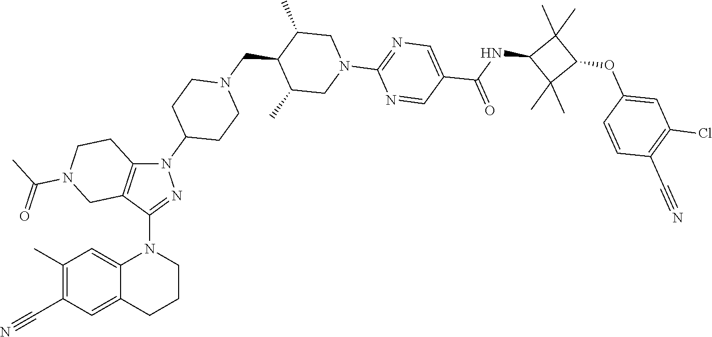 [SMILES: CC(=O)N1CCc2c(c(N3CCCc4cc(C#N)c(C)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 457 | 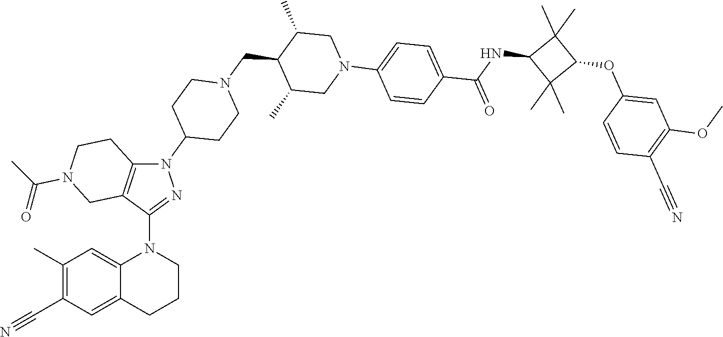 [SMILES: COc1cc(O[C@H]2C(C)(C)[C@H](NC(=O)c3ccc(N4C[C@@H](C)[C@H](CN5CCC(n6nc(N7CCCc8cc(C#N)c(C)cc87)c7c6CCN(C(C)=O)C7)CC5)[C@@H](C)C4)cc3)C2(C)C)ccc1C#N] |
| 458 | 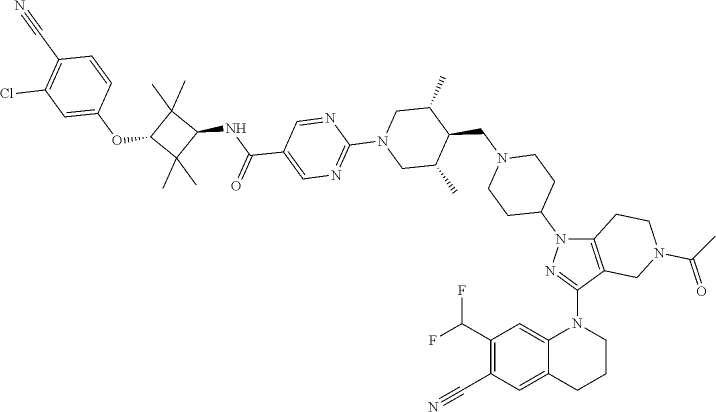 [SMILES: CC(=O)N1CCc2c(c(N3CCCc4cc(C#N)c(C(F)F)cc43)nn2C2CCN(C[C@H]3[C@H](C)CN(c4ncc(C(=O)N[C@H]5C(C)(C)[C@H](Oc6ccc(C#N)c(Cl)c6)C5(C)C)cn4)C[C@@H]3C)CC2)C1] |
| 459 | 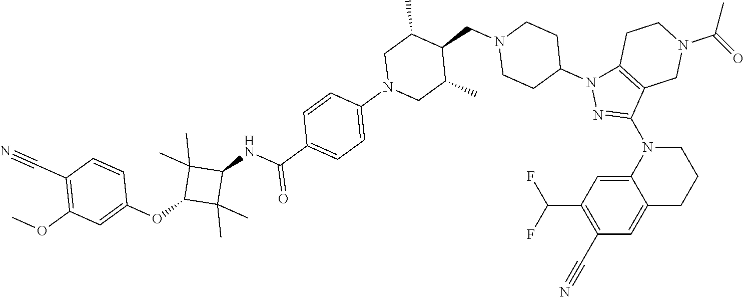 [SMILES: COc1cc(O[C@H]2C(C)(C)[C@H](NC(=O)c3ccc(N4C[C@@H](C)[C@H](CN5CCC(n6nc(N7CCCc8cc(C#N)c(C(F)F)cc87)c7c6CCN(C(C)=O)C7)CC5)[C@@H](C)C4)cc3)C2(C)C)ccc1C#N] |
| 460 | 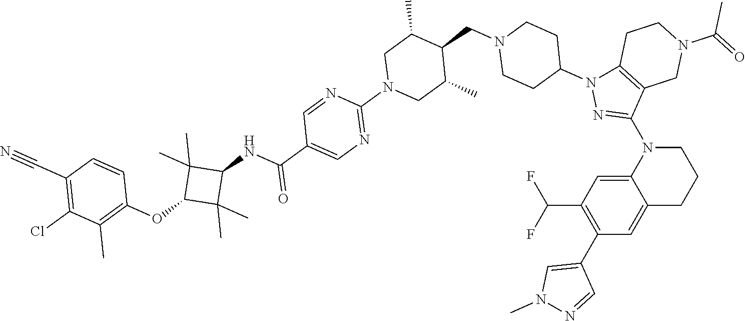 [SMILES: CC(=O)N1CCc2c(c(N3CCCc4cc(-c5cnn(C)c5)c(C(F)F)cc43)nn2C2CCN(C[C@H]3[C@H](C)CN(c4ncc(C(=O)N[C@H]5C(C)(C)[C@H](Oc6ccc(C#N)c(Cl)c6C)C5(C)C)cn4)C[C@@H]3C)CC2)C1] |
| 461 | 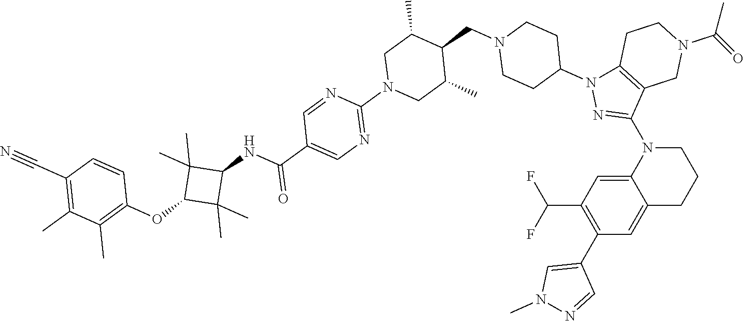 [SMILES: CC(=O)N1CCc2c(c(N3CCCc4cc(-c5cnn(C)c5)c(C(F)F)cc43)nn2C2CCN(C[C@H]3[C@H](C)CN(c4ncc(C(=O)N[C@H]5C(C)(C)[C@H](Oc6ccc(C#N)c(C)c6C)C5(C)C)cn4)C[C@@H]3C)CC2)C1] |
| 462 | 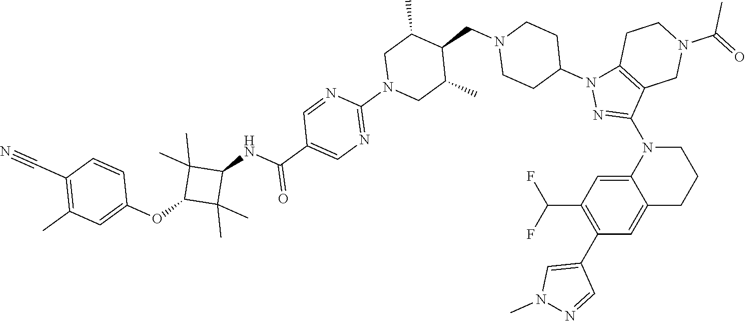 [SMILES: CC(=O)N1CCc2c(c(N3CCCc4cc(-c5cnn(C)c5)c(C(F)F)cc43)nn2C2CCN(C[C@H]3[C@H](C)CN(c4ncc(C(=O)N[C@H]5C(C)(C)[C@H](Oc6ccc(C#N)c(C)c6)C5(C)C)cn4)C[C@@H]3C)CC2)C1] |
| 463 | 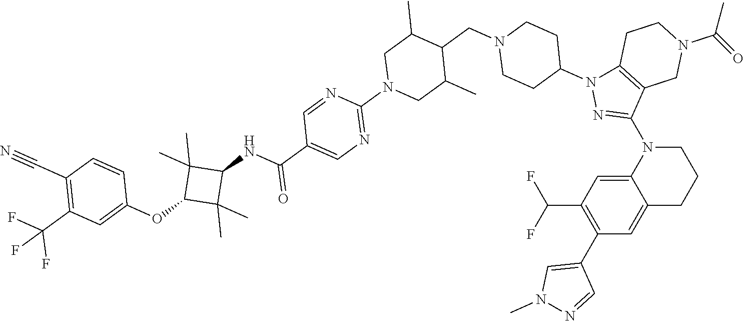 [SMILES: CC(=O)N1CCc2c(c(N3CCCc4cc(-c5cnn(C)c5)c(C(F)F)cc43)nn2C2CCN(CC3C(C)CN(c4ncc(C(=O)N[C@H]5C(C)(C)[C@H](Oc6ccc(C#N)c(C(F)(F)F)c6)C5(C)C)cn4)CC3C)CC2)C1] |
| 464 | 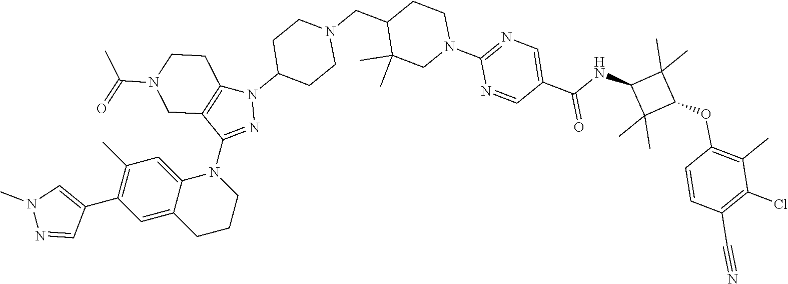 [SMILES: CC(=O)N1CCc2c(c(N3CCCc4cc(-c5cnn(C)c5)c(C)cc43)nn2C2CCN(CC3CCN(c4ncc(C(=O)N[C@H]5C(C)(C)[C@H](Oc6ccc(C#N)c(Cl)c6C)C5(C)C)cn4)CC3(C)C)CC2)C1] |
| 465 | 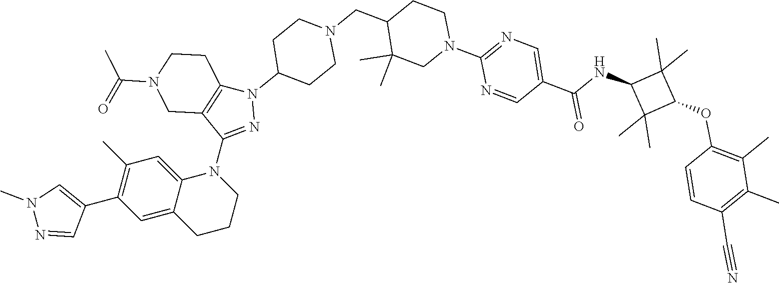 |
| 466 | 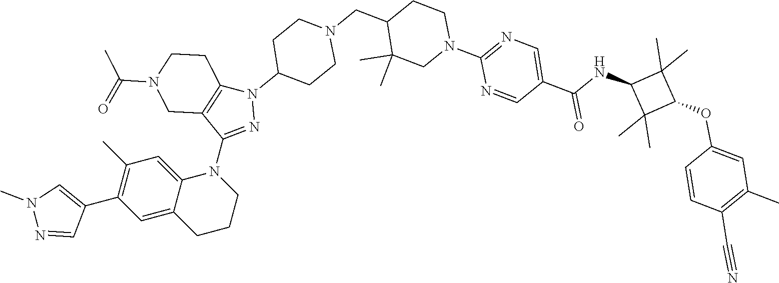 |
| 467 | 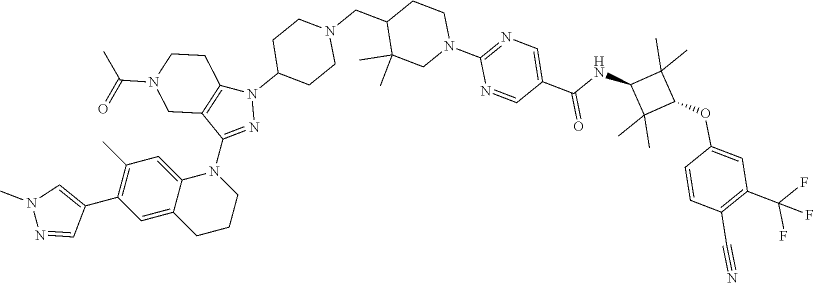 [SMILES: CC(=O)N1CCc2c(c(N3CCCc4cc(-c5cnn(C)c5)c(C)cc43)nn2C2CCN(CC3CCN(c4ncc(C(=O)N[C@H]5C(C)(C)[C@H](Oc6ccc(C#N)c(C(F)(F)F)c6)C5(C)C)cn4)CC3(C)C)CC2)C1] |
| 468 | 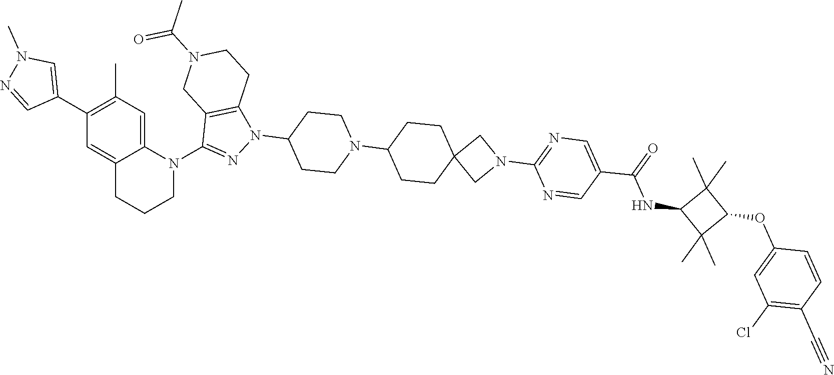 [SMILES: CC(=O)N1CCc2c(c(N3CCCc4cc(-c5cnn(C)c5)c(C)cc43)nn2C2CCN(C3CCC4(CC3)CN(c3ncc(C(=O)N[C@H]5C(C)(C)[C@H](Oc6ccc(C#N)c(Cl)c6)C5(C)C)cn3)C4)CC2)C1] |
| 469 | 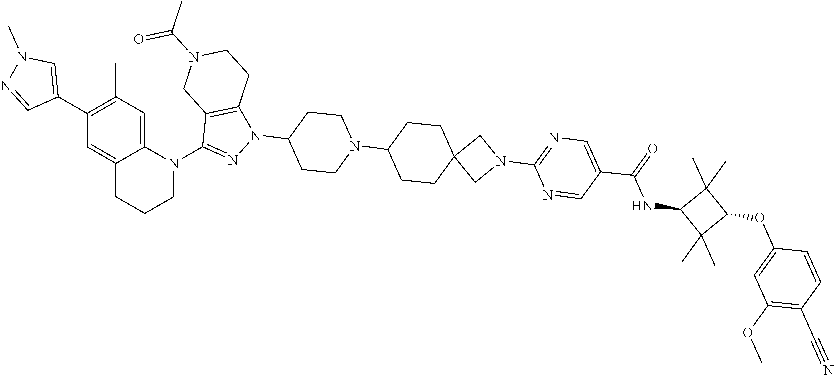 [SMILES: COc1cc(O[C@H]2C(C)(C)[C@H](NC(=O)c3cnc(N4CC5(CCC(N6CCC(n7nc(N8CCCc9cc(-c%10cnn(C)c%10)c(C)cc98)c8c7CCN(C(C)=O)C8)CC6)CC5)C4)nc3)C2(C)C)ccc1C#N] |
| 470 | 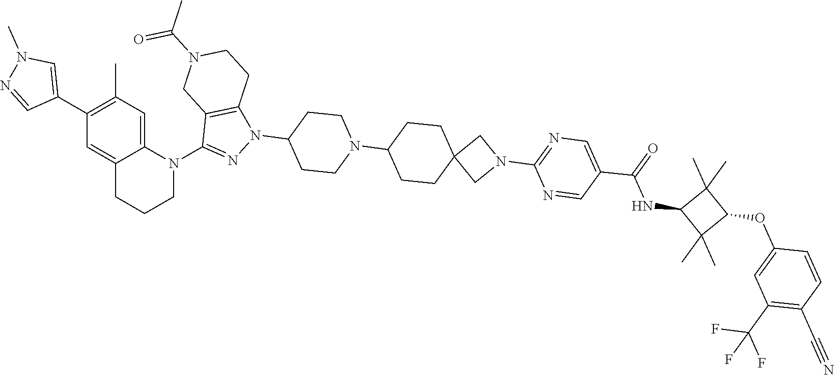 [SMILES: CC(=O)N1CCc2c(c(N3CCCc4cc(-c5cnn(C)c5)c(C)cc43)nn2C2CCN(C3CCC4(CC3)CN(c3ncc(C(=O)N[C@H]5C(C)(C)[C@H](Oc6ccc(C#N)c(C(F)(F)F)c6)C5(C)C)cn3)C4)CC2)C1] |
| 471 | 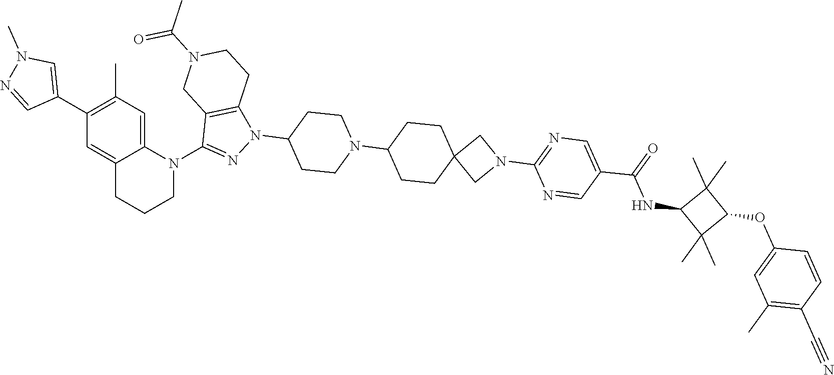 [SMILES: CC(=O)N1CCc2c(c(N3CCCc4cc(-c5cnn(C)c5)c(C)cc43)nn2C2CCN(C3CCC4(CC3)CN(c3ncc(C(=O)N[C@H]5C(C)(C)[C@H](Oc6ccc(C#N)c(C)c6)C5(C)C)cn3)C4)CC2)C1] |
| 472 | 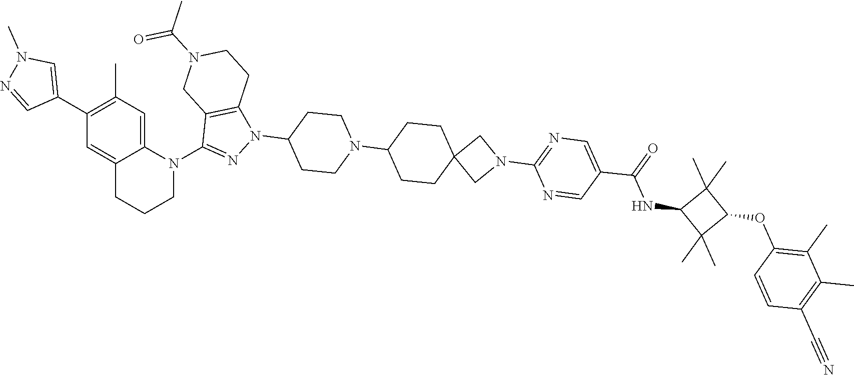 [SMILES: CC(=O)N1CCc2c(c(N3CCCc4cc(-c5cnn(C)c5)c(C)cc43)nn2C2CCN(C3CCC4(CC3)CN(c3ncc(C(=O)N[C@H]5C(C)(C)[C@H](Oc6ccc(C#N)c(C)c6C)C5(C)C)cn3)C4)CC2)C1] |
| 473 | 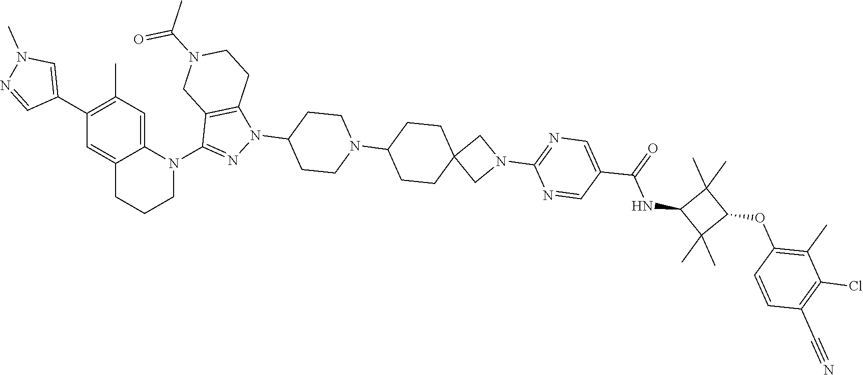 [SMILES: CC(=O)N1CCc2c(c(N3CCCc4cc(-c5cnn(C)c5)c(C)cc43)nn2C2CCN(C3CCC4(CC3)CN(c3ncc(C(=O)N[C@H]5C(C)(C)[C@H](Oc6ccc(C#N)c(Cl)c6C)C5(C)C)cn3)C4)CC2)C1] |

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting a compound disclosed herein with a base. In some embodiments, the compound disclosed herein is acidic and is reacted with abase. In such situations, an acidic proton of the compound disclosed herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure disclosed herein, as well as active metabolites of these compounds having the same type of activity.

In some embodiments, sites on the organic radicals (e.g. alkyl groups, aromatic rings) of compounds disclosed herein are susceptible to various metabolic reactions. Incorporation of appropriate substituents on the organic radicals will reduce, minimize, or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds disclosed herein possess one or more stereocenters and each stereocenter exists independently in either the R or S configuration. In some embodiments, the compound disclosed herein exists in the R configuration. In some embodiments, the compound disclosed herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

### Certain Terminology

Unless otherwise stated, the following terms used in this application have the definitions given below. The use of the term “including” as well as other forms, such as “include”, “includes,” and “included,” is not limiting.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “acyl,” as used herein refers to the group —C(═O)—R, where R is alkyl, alkenyl, alkynyl, aryl, cycloalkyl, heteroaryl, heterocycle, or any other moiety were the atom attached to the carbonyl is carbon. An “acetyl” group refers to a —C(═O)CH₃ group.

The term “alkenyl,” as used herein refers to a straight-chain or branched-chain hydrocarbon radical having one or more double bonds and containing from 2 to 20 carbon atoms. In certain embodiments, alkenyl includes 2 to 6 carbon atoms. The term “alkenylene” refers to a divalent alkenyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)=CH₂, —CH═CHCH₃, —C(CH₃)=CHCH₃, and —CH₂CH═CH₂.

The term “alkoxy” refers to a (alkyl)-O— group, where alkyl is as defined herein. In some embodiments, the alkoxy group is a C₁-C₆alkoxy, which refers to a (C₁-C₆alkyl)-O— group. Examples of alkyl groups include methoxy, ethoxy, n-propoxy, isopropoxy, n-butoxy, iso-butoxy, sec-butoxy, tert-butoxy, and the like.

An “alkyl” group refers to an aliphatic hydrocarbon group. In some embodiments, the alkyl is a straight-chain or branched-chain aliphatic hydrocarbon group containing from 1 to 20 carbon atoms. In certain embodiments, alkyl includes 1 to 10 carbon atoms. In further embodiments, the alkyl includes 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. In some embodiments, an alkyl is a C₁-C₆alkyl. In one aspect the alkyl is methyl, ethyl, propyl, iso-propyl, n-butyl, iso-butyl, sec-butyl, or t-butyl. The term “alkylene” refers to a divalent alkyl, such as methylene (—CH₂—). In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like.

The term “amino,” as used herein refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. In one aspect, “amino” as used herein refers to an —NH₂ group.

The term “alkynyl,” as used herein refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH. The term “alkynylene” refers to a carbon-carbon triple bond attached at two positions such as ethynylene (—C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The term “aromatic” refers to a planar ring having a delocalized it-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

The term “aryl” as used herein means a carbocyclic aromatic system containing one, two or three rings wherein such polycyclic ring systems are fused together. The term “aryl” embraces aromatic groups such as phenyl, naphthyl, anthracenyl, and phenanthryl. In one aspect, aryl is phenyl or a naphthyl. In some embodiments, an aryl is a phenyl. In some embodiments, an aryl is a phenyl, naphthyl, indanyl, indenyl, or tetrahyodronaphthyl. In some embodiments, an aryl is a C₆-C₁₀aryl. Depending on the structure, an aryl group is a monoradical or a diradical (i.e., an arylene group).

The terms “benzo” and “benz,” as used herein refer to fused bicyclic or polyclic ring system that is formed with benzene as one of the rings. Examples include benzofuran, benzothiophene, and benzimidazole.

The term “cycloalkyl,” as used herein refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In some embodiments, cycloalkyl groups include groups having from 3 to 10 ring atoms. In certain embodiments, said cycloalkyl will comprise from 5 to 7 carbon atoms. In certain embodiments, said cycloalkyl will comprise from 3 to 6 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantly, and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane. In some embodiments, acycloalkyl is a C₃-C₆cycloalkyl. In some embodiments, a cycloalkyl is a C₃-C₄cycloalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofiranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. In some embodiments, the term “heteroaryl,” as used herein refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroayl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, and the like. Exemplary tricyclic heterocyclic groups include carbazolyl, benzidolyl, phenanthrolinyl, dibenzofuranyl, acridinyl, phenanthridinyl, xanthenyl, and the like. In some embodiments, a heteroaryl contains 0-4 N atoms in the ring. In some embodiments, a heteroaryl contains 1-4 N atoms in the ring. In some embodiments, a heteroaryl contains 1 O atom in the ring. In some embodiments, a heteroaryl contains 1 S atom in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, the term “heterocycloalkyl” as used herein each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-2 N atoms in the ring. In some embodiments, a heterocycloalkyl contains 1-2 O atoms. In some embodiments, a heterocycloalkyl contains 1 S atom. In some embodiments, a heterocycloalkyl contains 0-2 N atoms, 0-2 O atoms and 0-1 S atoms in the ring.

The term “carbamate,” as used herein refers to an ester of carbamic acid (—NHCOO—) which may be attached to the parent molecular moiety from either the nitrogen or acid end, and which may be optionally substituted as defined herein.

The term “carboxyl” or “carboxy,” as used herein, refers to —C(═O)OH or the corresponding “carboxylate” anion, such as is in a carboxylic acid salt.

The term “cyano,” as used herein refers to —CN.

The term “ester,” as used herein refers to a carboxy group bridging two moieties linked at carbon atoms.

The term “ether,” as used herein refers to an oxy group bridging two moieties linked at carbon atoms.

The term “halo,” or “halogen,” as used herein refers to fluorine, chlorine, bromine, or iodine. In some embodiments, halo is fluoro, chloro, or bromo.

The term “haloalkyl,” as used herein refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—), and the like. In one aspect, a haloalkyl is a C₁-C₆haloalkyl. In another aspect, a haloalkyl is a C₁-C₄haloalkyl.

The term “haloalkoxy,” as used herein refers to a haloalkyl group attached to the parent molecular moiety through an oxygen atom. In one aspect, the haloalkoxy is a C₁-C₆haloalkoxy, which refers to a (C₁-C₆haloalkyl)-O— group. In another aspect, the haloalkoxy is a C₁-C₄haloalkoxy, which refers to a (C₁-C₄haloalkyl)-O— group.

The term “heteroalkyl” refers to an alkyl wherein 1 or more carbon atoms are replaced with a heteroatom. In some embodiments, “heteroalkyl” refers to an alkyl wherein 1 or more carbon atoms are replaced with one or more heteroatoms that are independently selected from NH, —N(alkyl), O, S, S(═O) and S(═O)₂. The attachment of the heteroatom(s) to the remainder of the compound is at a carbon atoms of the heteroalkyl. In some embodiments, up to two heteroatoms may be consecutive, such as, for example, —CH₂—NH—OCH₃. In some embodiments, “heteroalkyl” is an “alkoxyalkyl”, “alkylthioalkyl”, or “alkylaminoalkyl”. “Alkoxyalkyl” refers to an alkyl in which one hydrogen atom is replaced by an alkoxy group, as defined herein. In some embodiments, an alkoxyalkyl is a (C₁-C₆alkoxy)-C₁-C₆alkyl. Typical alkoxyalkyl groups include, but are not limited to, —CH₂OCH₃, —CH₂CH₂OCH₃, —CH₂CH₂CH₂OCH₃, —CH₂CH₂CH₂CH₂OCH₃, —CH₂OCH₂CH₃, —CH₂CH₂OCH₂CH₃, —CH₂CH₂CH₂OCH₂CH₃, —CH₂CH₂CH₂CH₂OCH₂CH₃, and the like. “Alkylthioalkyl” refers to an alkyl in which one hydrogen atom is replaced by an alkylthio group, as defined herein. In some embodiments, an alkoxyalkyl is a (C₁-C₆ alkylthio)-C₁-C₆alkyl. Typical alkoxyalkyl groups include, but are not limited to, —CH₂SCH₃, —CH₂CH₂SCH₃, —CH₂CH₂CH₂SCH₃, —CH₂CH₂CH₂CH₂SCH₃, —CH₂SCH₂CH₃, —CH₂CH₂SCH₂CH₃, —CH₂CH₂CH₂SCH₂CH₃, —CH₂CH₂CH₂CH₂SCH₂CH₃, and the like. “Alkylaminoalkyl” refers to an alkyl in which one hydrogen atom is replaced by an alkylamino group, as defined herein. In some embodiments, an alkoxyalkyl is a (C₁-C₆alkylamino)-C₁-C₆alkyl. Typical alkoxyalkyl groups include, but are not limited to, —CH₂NHCH₃, —CH₂CH₂NHCH₃, —CH₂CH₂CH₂NHCH₃, —CH₂CH₂CH₂CH₂NHCH₃, —CH₂NHCH₂CH₃, —CH₂CH₂NHCH₂CH₃, —CH₂CH₂CH₂NHCH₂CH₃, —CH₂CH₂CH₂CH₂NHCH₂CH₃, and the like.

The term “hydroxy,” or “hydroxyl,” as used herein refers to —OH.

The term “hydroxyalkyl,” as used herein refers to a hydroxy group attached to the parent molecular moiety through an alkyl group. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

The phrase “linear chain of atoms” refers to the longest straight chain of atoms independently selected from carbon, nitrogen, oxygen and sulfur.

The term “nitro,” as used herein refers to —NO₂.

The term “oxo,” as used herein refers to ═O.

The terms “sulfonate,” “sulfonic acid,” and “sulfonic,” as used herein refer the —SO₃H group and its anion as the sulfonic acid is used in salt formation.

The term “sulfanyl,” as used herein refers to —S—.

The term “sulfinyl,” as used herein refers to —S(═O)—.

The term “sulfonyl,” as used herein refers to a —S(═O)₂—, —S(═O)₂R, or —S(═O)₂R— group, with R as defined herein.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

In some embodiments, the term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” or “disorder” as used herein refers to any condition that impairs the normal functioning of the body, such as a functional abnormality or disturbance that impairs normal functioning.

The term “combination therapy” means the administration of two or more therapeutic agents to treat a therapeutic condition or disorder described in the present disclosure.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease or may involve prevention of disease progression. Prevention of diseases may also mean prevention of progression of a disease to a later stage of the disease.

The term “patient” is generally synonymous with the term “subject” and includes all mammals including humans. Examples of patients include humans, non-human primates such as chimpanzees, and other apes and monkey species; livestock such as cows, goats, sheep, pigs, and rabbits, and companion animals such as dogs, cats, rabbits, and horses. Preferably, the patient is a human.

### Pharmaceutical Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro et. al.).

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds provided herein or a salt, solvate, or hydrate thereof can be used as active ingredients in pharmaceutical compositions. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety of factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents.

Aqueous formulations suitable for oral use can be prepared by dissolving or suspending the active component in water and adding suitable colorants, flavors, stabilizing and thickening agents, as desired.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds provided herein may be formulated as ointments, creams, or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

### Methods of Dosing and Treatment Regimens

In one embodiment, the compounds disclosed herein, or a pharmaceutically acceptable salt thereof, are used in the preparation of medicaments for the treatment of diseases or conditions in a mammal. Methods for treating any of the diseases or conditions described herein in a mammal in need of such treatment, involves administration of pharmaceutical compositions that include at least one compound disclosed herein, or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound disclosed herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound disclosed herein, or a pharmaceutically acceptable salt thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound disclosed herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound disclosed herein, or a pharmaceutically acceptable salt thereof, in combination with one or more other therapeutic agents.

As used above, and throughout the description of the invention, the following abbreviations, unless otherwise indicated, shall be understood to have the following meanings:
ACN or MeCN acetonitrile
Ac acetyl
Ac₂O acetic anhydride
AcOH acetic acid
AIBN azobisisobutyronitrile
BINAP 2,2′-bis(diphenylphosphino)-1,1′-binaphthalene
Bn benzyl
BOC or Boc tert-butyl carbamate
Boc₂O di-tert-butyl decarbonate or Boc anhydride
BPO benzoyl peroxide
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
t-Bu tert-butyl
C Phos Pd G3 [(2-Dicyclohexylphosphino-2′,6′-bis(N,N-dimethylamino)-1,1′-biphenyl)-2-(2′-amino-1,1′-biphenyl)]palladium(II) methanesulfonate
Cy cyclohexyl
DBA or dba dibenzylideneacetone
DBU 1,8-diazabicyclo[5.4.0]undec-7-ene
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
Dppf 1,1′-bis(diphenylphosphino)ferrocene
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
FA formic acid
HATU 1-[bis(dimethylamino)methylene]-1H-1,2,3-triazolo[4,5-b]pyridinium 3-oxid hexafluorophosphate
HPLC high performance liquid chromatography
H₂O water
K₂CO₃ potassium carbonate
LAH lithium aluminum anhydride
LCMS liquid chromatography mass spectrometry
Me methyl
MeOH methanol
MS mass spectrometry
MsCl mesyl chloride
Na₂CO₃ sodium carbonate
NaBH(OAc)₃ sodium triacetoxyborohydride
NaH sodium hydride
Na₂SO₄ sodium sulfate
NBS N-bromo succinamide
NMP N-methyl-pyrrolidin-2-one
NMR nuclear magnetic resonance
Pd(dppf)Cl₂ (1,1′-Bis(diphenylphosphino)ferrocene)palladium(II) dichloride
Ph phenyl
Pin pinacolato
Prep-HPLC preparative high performance liquid chromatography
i-Pr iso-propyl
RP-HPLC reverse phase-high pressure liquid chromatography
RuPhos Pd G3 (2-Dicyclohexylphosphino-2′,6′-diisopropoxy-1,1′-biphenyl)[2-(2′-amino-1,1′-biphenyl)]palladium(II) methanesulfonate
TBS tert-butyldimethylsilyl
TFA trifluoroacetic acid
TEA triethylamine
THF tetrahydrofuran
TsCl tosyl chloride
TLC thin layer chromatography

### EXAMPLES

The following examples are provided for illustrative purposes only and not to limit the scope of the claims provided herein.

Compound A was prepared according to the procedure described in WO 2022/42707 A1.

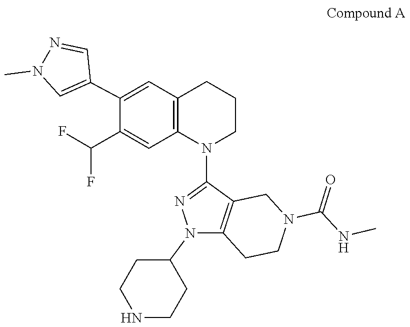

Compound B was prepared according to the procedure described in Journal of Medicinal Chemistry, 2023, 66, 7, 4784-4801.

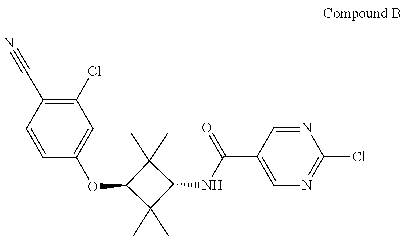 [SMILES: CC1(C)[C@H](NC(=O)c2cnc(Cl)nc2)C(C)(C)[C@H]1Oc1ccc(C#N)c(Cl)c1]

Compound C was prepared according to the following procedure:

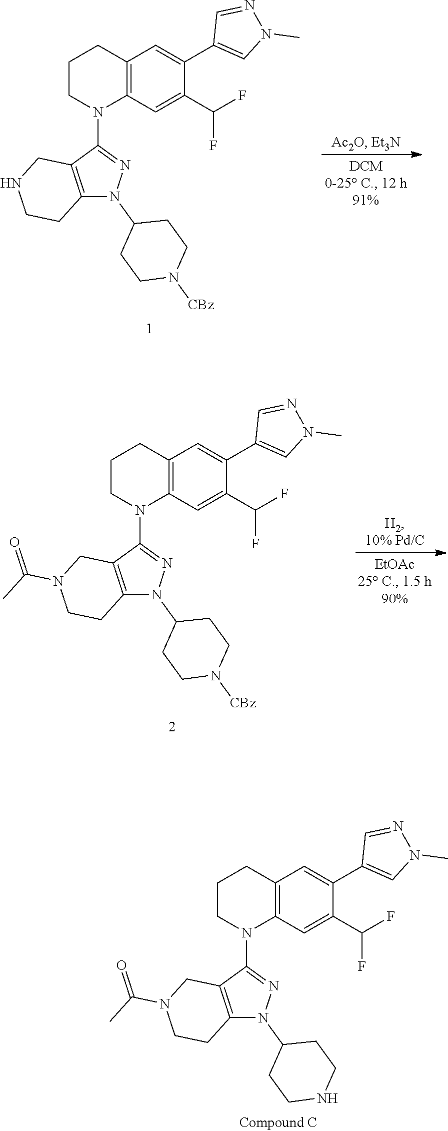

1 was prepared according to the procedure described in WO 2022/42707 A1.

To a solution of benzyl 4-[3-[7-(difluoromethyl)-6-(1-methylpyrazol-4-yl)-3,4-dihydro-2H-quinolin-1-yl]-4,5,6,7-tetrahydropyrazolo[4,3-c]pyridin-1-yl]piperidine-1-carboxylate (0.250 g, 349 μmol, 1.0 eq, TFA) in DCM (0.5 mL) was added TEA (106 mg, 1.05 mmol, 146 μL, 3.0 eq) and Ac₂O (53.5 mg, 524 μmol, 49.2 μL, 1.5 eq) at 0° C. The mixture was stirred at 25° C. for 12 h. The mixture was poured into ice-water (20 mL). The aqueous phase was extracted with dichloromethane (15 mL×3). The combined organic phase was washed with brine (10 mL×2), dried over anhydrous sodium sulfate, filtered and concentrated under vacuum. The residue was purified by prep-TLC (silica, dichloromethane/methyl alcohol=10/1). Compound benzyl 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (0.21 g, 316 μmol, 91% yield) was obtained as a light yellow solid. LC-MS: MS (ESI⁺): t_{R}=0.980 min, m/z=644.3 [M+H⁺]

Pd/C (0.110 g, 10% Pd on carbon, w/w) was added into a 100 mL single-necked round bottom flask under N₂, and then EtOAc (10 mL) was added at 25° C. under N₂. After addition, benzyl 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (0.210 g, 326 μmol, 1.0 eq) was added under N₂. The suspension was degassed under vacuum and purged with H₂ several times. The mixture was stirred under H₂ (15 psi) at 25° C. for 1.5 h. The reaction mixture was filtered and washed with MeOH (20 mL×3). The collected filtrate was concentrated to give a residue. The residue was used for the next step without further purification. Compound 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (0.15 g, 294 μmol, 90% yield) was obtained as a white solid. LC-MS: MS (ESI⁺): t_{R}=0.429 min, m/z=510.3 [M+H⁺]

Compound E was prepared according to the procedure described in WO 2017/205538 A1.

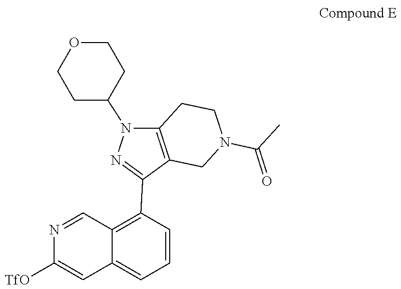 [SMILES: CC(=O)N1CCc2c(c(-c3cccc4cc(OS(=O)(=O)C(F)(F)F)ncc34)nn2C2CCOCC2)C1]

Compound F was prepared according to the procedure described in Journal of Medicinal Chemistry, 2023, 66, 7, 4784-4801.

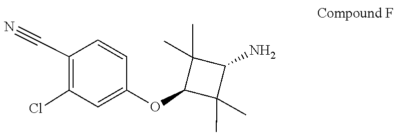 [SMILES: CC1(C)[C@H](N)C(C)(C)[C@H]1Oc1ccc(C#N)c(Cl)c1]

Compound G was prepared according to the following procedure:

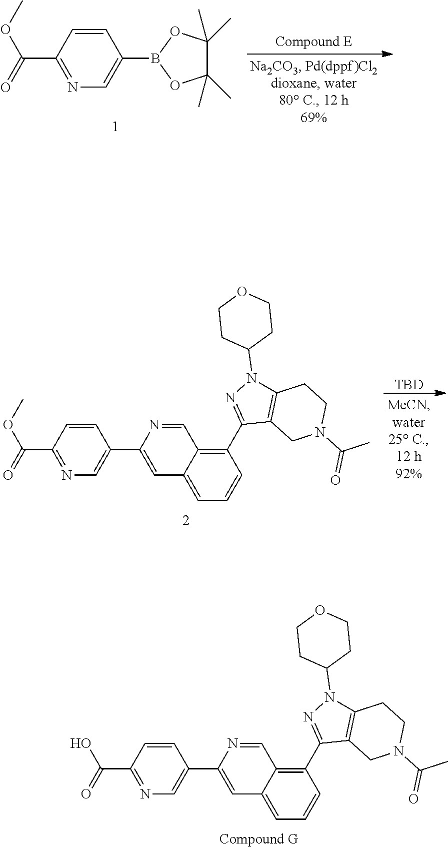 [SMILES: COC(=O)c1ccc(B2OC(C)(C)C(C)(C)O2)cn1 ; COC(=O)c1ccc(-c2cc3cccc(-c4nn(C5CCOCC5)c5c4CN(C(C)=O)CC5)c3cn2)cn1 ; "TBD" ; CC(=O)N1CCc2c(c(-c3cccc4cc(-c5ccc(C(=O)O)nc5)ncc34)nn2C2CCOCC2)C1]

To a solution of methyl 5-(4,4,5,5-tetramethyl-1,3,2-dioxaborolan-2-yl)pyridine-2-carboxylate (0.15 g, 570.13 μmol, 1 eq) and Na₂CO₃ (120.86 mg, 1.14 mmol, 2 eq) and Pd(dppf)Cl₂ (41.72 mg, 57.01 μmol, 0.1 eq) in dioxane (5 mL) and H₂O (0.5 mL) was added [8-(5-acetyl-1-tetrahydropyran-4-yl-6,7-dihydro-4H-pyrazolo[4,3-c]pyridin-3-yl)-3-isoquinolyl]trifluoromethanesulfonate (299 mg, 570 μmol, 1.0 eq). The mixture was stirred at 80° C. for 12 h. The mixture was poured into H₂O (100 mL) and extracted with ethyl acetate (100 mL×2). The combined organics were washed with brine (200 mL×2), dried with anhydrous Na₂SO₄, filtered, and concentrated in vacuum. The residue was purified by column chromatography (silica, petroleum ether/ethyl acetate=50/1 to 0/1, DCM/MeOH=50/1 to 20/1). Compound methyl 5-[8-(5-acetyl-1-tetrahydropyran-4-yl-6,7-dihydro-4H-pyrazolo[4,3-c]pyridin-3-yl)-3-isoquinolyl]pyridine-2-carboxylate (0.20 g, 390.95 μmol, 68% yield) was obtained as a yellow solid. LC-MS: MS (ESI⁺): t_{R}=0.468 min, m/z=512.2 [M+H⁺]

To a solution of methyl 5-[8-(5-acetyl-1-tetrahydropyran-4-yl-6,7-dihydro-4H-pyrazolo[4,3-c]pyridin-3-yl)-3-isoquinolyl]pyridine-2-carboxylate (0.20 g, 391 μmol, 1.0 eq) in MeCN (3 mL) and H₂O (1 mL) was added 3,4,6,7,8,9-hexahydro-2H-pyrimido[1,2-a]pyrimidine (109 mg, 782 μmol, 2.0 eq). The mixture was stirred at 25° C. for 12 h. The mixture was added 1 M HCl (0.10 mL) and extracted with DCM (100 mL×2). The combined organic phase was washed with brine (100 mL×2), dried with anhydrous sodium sulfate, filtered and concentrated in vacuum. The residue was purified by prep-HPLC (column: Phenomenex luna C18 150*25 mm*10 um; mobile phase: [water(HCl)-ACN]; gradient: 13%-43% B over 10 min). Compound 5-[8-(5-acetyl-1-tetrahydropyran-4-yl-6,7-dihydro-4H-pyrazolo[4,3-c]pyridin-3-yl)-3-isoquinolyl]pyridine-2-carboxylic acid (0.12 g, 241 μmol, 62% yield) was obtained as a yellow solid. LC-MS: MS (ESI⁺): t_{R}=0.477 min, m/z=498.3 [M+Na⁺]

Compound H was prepared according to the following procedure:

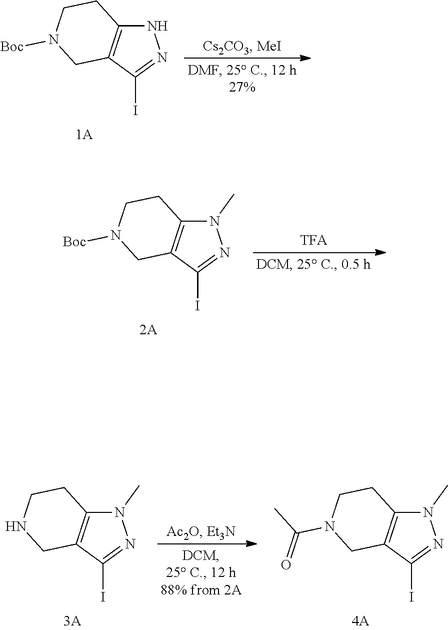 [SMILES: CC(C)(C)OC(=O)N1CCc2[nH]nc(I)c2C1 ; Cn1nc(I)c2c1CCN(C(=O)OC(C)(C)C)C2 ; Cn1nc(I)c2c1CCNC2 ; CC(=O)N1CCc2c(c(I)nn2C)C1]

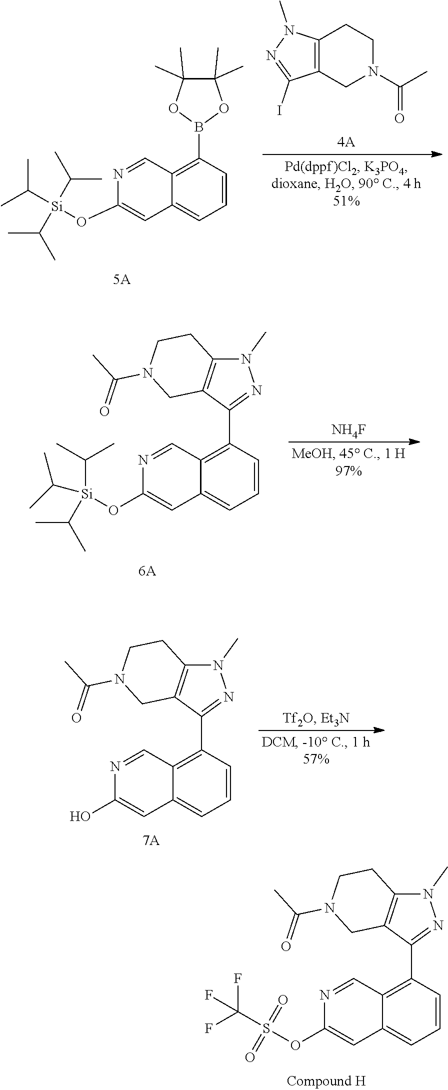

Synthesis of 5A was reported in WO2021/231174 A1.

A mixture of tert-butyl 3-iodo-1,4,6,7-tetrahydropyrazolo[4,3-c]pyridine-5-carboxylate (15.0 g, 42.96 mmol, 1 eq), Cs₂CO₃ (41.9 g, 128.88 mmol, 3.0 eq) in DMF (150 mL) was degassed and purged with N₂ for 3 times, and then added Mel (9.15 g, 64.44 mmol, 4.01 mL, 1.5 eq), the mixture was stirred at 25° C. for 1 2 h under N₂. The reaction mixture was quenched by addition water 1000 mL, and then extracted with ethyl acetate (300 mL×3). The combined organic layers were washed with brine (300 mL×3), dried over Na₂SO₄, filtered and concentrated under reduced pressure to give a residue. The residue was purified by column chromatography (SiO₂, Petroleum ether/Ethyl acetate=10/1 to 2/1). Compound tert-butyl 3-iodo-1-methyl-6,7-dihydro-4H-pyrazolo[4,3-c]pyridine-5-carboxylate (4.3 g, 11.84 mmol, 27% yield) was obtained as a white solid.

¹H NMR (400 MHz, DMSO-d₆): δ=4.09 (s, 2H), 3.69 (s, 3H), 3.59 (t, J=5.6 Hz, 2H), 2.64 (t, J=5.6 Hz, 2H), 1.41 (s, 9H)

LC-MS: MS (ESI⁺): t_{R}=0.521 min, m/z=364.1 [M+H⁺]

To a solution of tert-butyl 3-iodo-1-methyl-6,7-dihydro-4H-pyrazolo[4,3-c]pyridine-5-carboxylate (4.30 g, 11.84 mmol, 1.0 eq) in DCM (20 mL) was added TFA (15.35 g, 134.62 mmol, 10 mL, 11.3 eq). The mixture was stirred at 25° C. for 0.5 h. The reaction mixture was concentrated in vacuo to give the crude product. Compound 3-iodo-1-methyl-4,5,6,7-tetrahydropyrazolo[4,3-c]pyridine (4.46 g) was obtained as a white solid and directly used in the next step without further purification.

LC-MS: MS (ESI⁺): t_{R}=0.218 min, m/z=263.1 [M+H⁺]

To a solution of 3-iodo-1-methyl-4,5,6,7-tetrahydropyrazolo[4,3-c]pyridine (4.46 g, 11.83 mmol, 1.0 eq) in DCM (40 mL) was added Et₃N (3.59 g, 35.48 mmol, 4.94 mL, 3.0 eq), Ac₂O (1.81 g, 17.74 mmol, 1.67 mL, 1.5 eq). The mixture was stirred at 25° C. for 12 h. The reaction mixture was concentrated in vacuo to give the crude product. The residue was purified by column chromatography (SiO₂, Petroleum ether/Ethyl acetate=10/1 to 0/1). Compound 1-(3-iodo-1-methyl-6,7-dihydro-4H-pyrazolo[4,3-c]pyridin-5-yl)ethanone (3.20 g, 10.49 mmol, 88% yield over two steps) was obtained as a white solid.

LC-MS: MS (ESI⁺): t_{R}=0.378 min, m/z=306.1 [M+H⁺]

A mixture of 1-(3-iodo-1-methyl-6,7-dihydro-4H-pyrazolo[4,3-c]pyridin-5-yl)ethanone (3.00 g, 9.83 mmol, 1.0 eq), triisopropyl-[[8-(4,4,5,5-tetramethyl-1,3,2-dioxaborolan-2-yl)-3-isoquinolyl]oxy]silane (7.00 g, 11.46 mmol, 1.1 eq), Pd(dppf)Cl₂ (359 mg, 491.62 μmol, 0.05 eq), K₃PO₄ (7.30 g, 34.41 mmol, 3.5 eq) and water (50 mL) in dioxane (80 mL) was degassed and purged with N₂ for 3 times, and then the mixture was stirred at 90° C. for 4 h under N₂. The reaction mixture was quenched by addition water 500 mL, and then extracted with DCM (100 mL×3). The combined organic layers were washed with brine (100 mL×3), dried over Na₂SO₄, filtered and concentrated under reduced pressure to give a residue. The residue was purified by column chromatography (SiO₂, dichloromethane:methanol=100/1 to 20/1). Compound 1-[1-methyl-3-(3-triisopropylsilyloxy-8-isoquinolyl)-6,7-dihydro-4H-pyrazolo[4,3-c]pyridin-5-yl]ethanone (2.43 g, 5.08 mmol, 51% yield) was obtained as a white solid.

LC-MS: MS (ESI⁺): t_{R}=0.893 min, m/z=479.3 [M+H⁺]

To a solution of 1-[1-methyl-3-(3-triisopropylsilyloxy-8-isoquinolyl)-6,7-dihydro-4H-pyrazolo[4,3-c]pyridin-5-yl]ethanone (2.43 g, 5.08 mmol, 1.0 eq) in MeOH (20 mL) was added NH₄F (1.88 g, 50.76 mmol, 10.0 eq). The mixture was stirred at 45° C. for 1 h. The reaction mixture was filtered and concentrated under reduced pressure to give a residue The residue was purified by column chromatography (SiO₂, dichloromethane:methanol=50/1 to 10/1). Compound 1-[3-(3-hydroxy-8-isoquinolyl)-1-methyl-6,7-dihydro-4H-pyrazolo[4,3-c]pyridin-5-yl]ethanone (1.6 g, 4.96 mmol, 97% yield) was obtained as a yellow solid.

1H NMR (400 MHz, DMSO-d₆): δ=10.97 (br s, 1H), 9.44-9.21 (m, 1H), 7.70-7.63 (m, 1H), 7.63-7.55 (m, 1H), 7.28-7.17 (m, 1H), 6.90 (s, 1H), 4.48 (s, 2H), 3.89-3.72 (m, 5H), 2.92-2.71 (m, 2H), 2.14-1.94 (m, 3H)

LC-MS: MS (ESI⁺): t_{R}=0.368 min, m/z=323.2 [M+H⁺]

To a solution of 1-[3-(3-hydroxy-8-isoquinolyl)-1-methyl-6,7-dihydro-4H-pyrazolo[4,3-c]pyridin-5-yl]ethanone (1.60 g, 4.96 mmol, 1.0 eq) in DCM (20 mL) was added Et₃N (1.51 g, 14.89 mmol, 2.07 mL, 3.0 eq), and added Tf₂O (2.10 g, 7.45 mmol, 1.23 mL, 1.5 eq). The mixture was stirred at −10° C. for 1 h. The reaction mixture was quenched by addition water 200 mL, and then extracted with DCM (50 mL×3). The combined organic layers were washed with brine (50 mL×3), dried over Na₂SO₄, filtered and concentrated under reduced pressure to give a residue. The residue was purified by column chromatography (SiO₂, dichloromethane:methanol=50/1 to 10/1). Compound [8-(5-acetyl-1-methyl-6,7-dihydro-4H-pyrazolo[4,3-c]pyridin-3-yl)-3-isoquinolyl]trifluoromethanesulfonate (1.3 g, 2.86 mmol, 57% yield) was obtained as a yellow solid.

1H NMR (400 MHz, DMSO-d₆): δ=9.82-9.76 (m, 1H), 8.21 (s, 1H), 8.14 (d, J=8.4 Hz, 1H), 8.04-7.96 (m, 1H), 7.82-7.71 (m, 1H), 4.56 (s, 2H), 3.89-3.76 (m, 5H), 2.93-2.74 (m, 2H), 2.16-1.98 (m, 3H)

LC-MS: MS (ESI⁺): t_{R}=0.521 min, m/z=455.2 [M+H⁺]

### Example 1: Synthesis of 1-[1-[2-[4-[5-[[3-(3-chloro-4-cyano-phenoxy)-2,2,4,4-tetramethyl-cyclobutyl]carbamoyl]pyrimidin-2-yl]piperazin-1-yl]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 109)

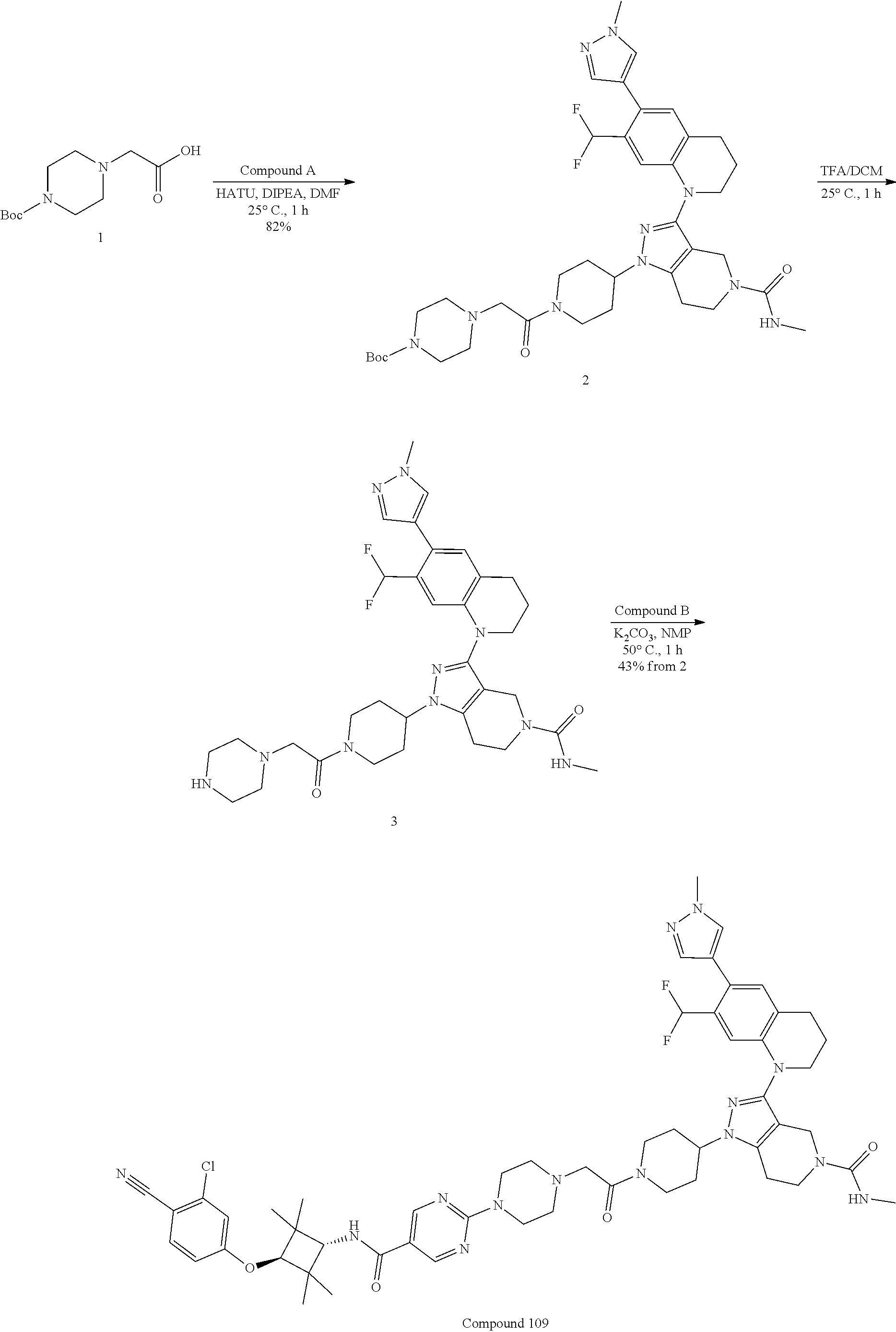 [SMILES: CC(C)(C)OC(=O)N1CCN(CC(=O)O)CC1 ; CNC(=O)N1CCc2c(c(N3CCCc4cc(-c5cnn(C)c5)c(C(F)F)cc43)nn2C2CCN(C(=O)CN3CCN(C(=O)OC(C)(C)C)CC3)CC2)C1 ; CNC(=O)N1CCc2c(c(N3CCCc4cc(-c5cnn(C)c5)c(C(F)F)cc43)nn2C2CCN(C(=O)CN3CCNCC3)CC2)C1 ; CNC(=O)N1CCc2c(c(N3CCCc4cc(-c5cnn(C)c5)c(C(F)F)cc43)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1]

To a mixture of 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (110 mg, 209 μmol, 1.0 eq) and 2-(4-tertbutoxycarbonylpiperazin-1-yl)acetic acid (76 mg, 314 μmol, 1.5 eq) in DMF (2 mL) was added DIPEA (135 mg, 1.05 mmol, 5.0 eq) and HATU (159 mg, 419 μmol, 2.0 eq), the mixture was stirred at 25° C. for 1 h. The reaction mixture was diluted with water (15 mL) and extracted with EtOAc (15 mL*3). The combined organic layers were concentrated to give a residue. The residue was purified by prep-HPLC (column: Phenomenex luna C18 150*25 mm*10 um; mobile phase: [water(FA)-ACN]; gradient: 18%-48% B over 10 min) to give tert-butyl 4-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethyl]piperazine-1-carboxylate (130 mg, 82% yield). LC-MS: MS (ESI⁺): t_{R}=0.828 min, m/z=751.5 [M+H⁺]

To a mixture of tert-butyl 4-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethyl]piperazine-1-carboxylate (130 mg, 173 μmol, 1.0 eq) in DCM (3 mL) was added TFA (1.54 g, 13.4 mmol, 1 mL, 77.7 eq), the mixture was stirred at 25° C. for 1 h. The mixture was concentrated to give a residue. Compound 3-[7-(difluoromethyl)-6-(1-methylpyrazol-4-yl)-3,4-dihydro-2H-quinolin-1-yl]-N-methyl-1-[1-(2-piperazin-1-ylacetyl)-4-piperidyl]-6,7-dihydro-4H-pyrazolo[4,3-c]pyridine-5-carboxamide (112 mg) was obtained as a brown solid and directly used in the next step without further purification. LC-MS: MS (ESI⁺): t_{R}=0.650 min, m/z=651.3 [M+H⁺]

To a mixture of 3-[7-(difluoromethyl)-6-(1-methylpyrazol-4-yl)-3,4-dihydro-2H-quinolin-1-yl]-N-methyl-1-[1-(2-piperazin-1-ylacetyl)-4-piperidyl]-6,7-dihydro-4H-pyrazolo[4,3-c]pyridine-5-carboxamide (112 mg) and 2-chloro-N-[3-(3-chloro-4-cyano-phenoxy)-2,2,4,4-tetramethyl-cyclobutyl]pyrimidine-5-carboxamide (72 mg, 172 μmol, 1.0 eq) in NMP (2 mL) was added K₂CO₃ (118 mg, 860 μmol, 5.0 eq), the mixture was stirred at 50° C. for 1 h. The mixture was filtered and concentrated to give a residue. The residue was purified by prep-HPLC (column: Phenomenex luna C18 150*25 mm*10 um; mobile phase: [water(FA)-ACN]; gradient: 31%-61% B over 10 min) to give 1-[1-[2-[4-[5-[[3-(3-chloro-4-cyano-phenoxy)-2,2,4,4-tetramethyl-cyclobutyl]carbamoyl]pyrimidin-2-yl]piperazin-1-yl]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 109, 170 mg, 43% yield over two steps). ¹H NMR (400 MHz, CDCl₃): δ=8.70 (s, 2H), 7.65-7.55 (m, 1H), 7.52 (s, 1H), 7.40 (s, 1H), 7.15-7.05 (m, 1H), 6.98-6.90 (m, 1H), 6.86 (s, 1H), 6.85-6.75 (m, 1H), 6.70-6.30 (m, 1H), 6.10-6.00 (m, 1H), 4.75-4.58 (m, 1H), 4.55-4.40 (m, 1H), 4.35-4.10 (m, 3H), 4.10-4.00 (m, 5H), 4.00-3.90 (m, 5H), 3.85-3.68 (m, 2H), 3.75-3.65 (m, 2H), 3.60-3.45 (m, 2H), 3.30-3.15 (m, 1H), 3.05-2.80 (m, 6H), 2.79-2.65 (m, 5H), 2.30-2.15 (m, 2H), 2.14-1.90 (m, 5H), 1.40-1.10 (m, 12H). LC-MS:MS (ESI⁺): t_{R}=1.933 min, m/z=1033.7 [M+H⁺].

### Example 2: Synthesis of 1-[1-[2-[1-[5-[[3-(3-chloro-4-cyano-phenoxy)-2,2,4,4-tetramethyl-cyclobutyl]carbamoyl]pyrimidin-2-yl]-4-piperidyl]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 101)

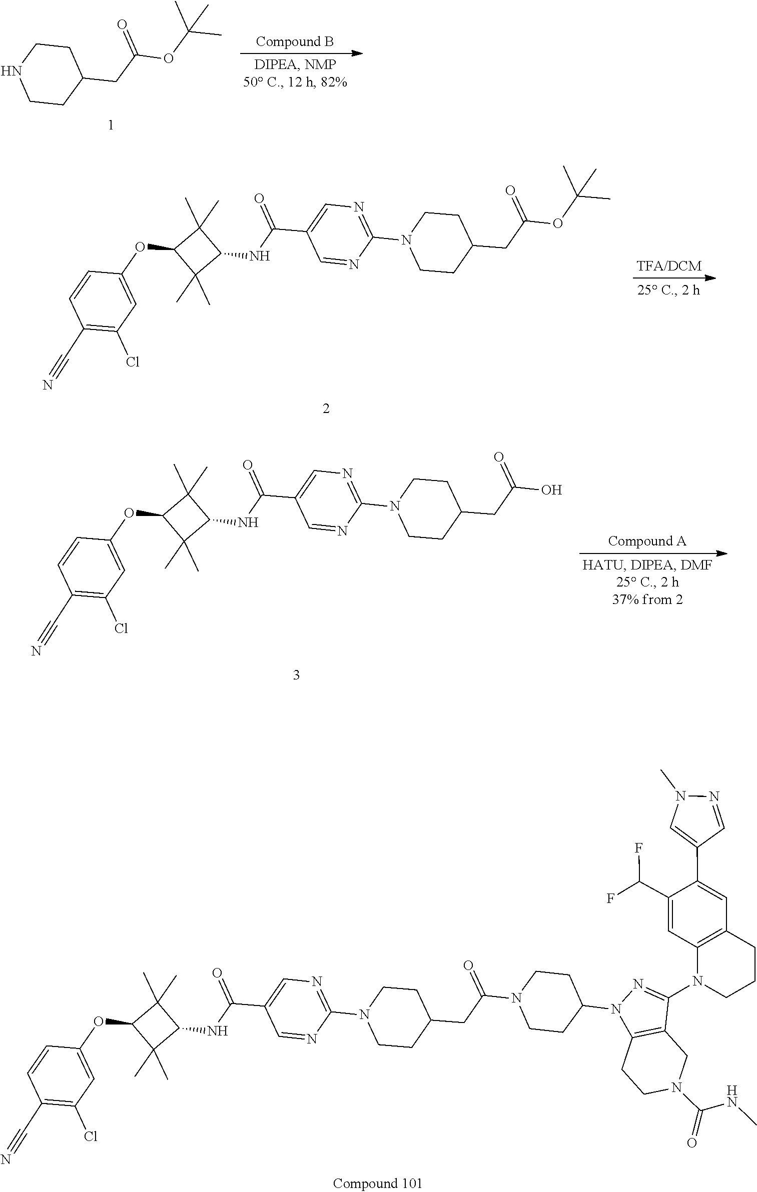 [SMILES: CC(C)(C)OC(=O)CC1CCNCC1 ; CC(C)(C)OC(=O)CC1CCN(c2ncc(C(=O)N[C@H]3C(C)(C)[C@H](Oc4ccc(C#N)c(Cl)c4)C3(C)C)cn2)CC1 ; CC1(C)[C@H](NC(=O)c2cnc(N3CCC(CC(=O)O)CC3)nc2)C(C)(C)[C@H]1Oc1ccc(C#N)c(Cl)c1 ; CNC(=O)N1CCc2c(c(N3CCCc4cc(-c5cnn(C)c5)c(C(F)F)cc43)nn2C2CCN(C(=O)CC3CCN(c4ncc(C(=O)N[C@H]5C(C)(C)[C@H](Oc6ccc(C#N)c(Cl)c6)C5(C)C)cn4)CC3)CC2)C1]

To a solution of tert-butyl 2-(4-piperidyl)acetate (48.0 mg, 238 μmol, 1.0 eq) in NMP (1 mL) was added DIPEA (92.5 mg, 715.47 μmol, 0.13 mL, 3.0 eq) and 2-chloro-N-[3-(3-chloro-4-cyano-phenoxy)-2,2,4,4-tetramethyl-cyclobutyl]pyrimidine-5-carboxamide (100 mg, 238 μmol, 1.0 eq). The mixture was stirred at 50° C. for 12 h. The reaction was diluted with water (10 mL) and the resulting mixture was extracted with DCM (20 mL×2). The combined organic layers were dried over Na₂SO₄ and concentrated to give the residue. The residue was purified by prep-TLC (silica, DCM/MeOH=10/1). Compound tert-butyl 2-[1-[5-[[3-(3-chloro-4-cyano-phenoxy)-2,2,4,4-tetramethyl-cyclobutyl]carbamoyl]pyrimidin-2-yl]-4-piperidyl]acetate (123 mg, 196 μmol, 82% yield) was obtained as a white solid. LC-MS: MS (ESI⁺): t_{R}=0.720 min, m/z=582.6 [M+H⁺]

To a solution of tert-butyl 2-[1-[5-[[3-(3-chloro-4-cyano-phenoxy)-2,2,4,4-tetramethyl-cyclobutyl]carbamoyl]pyrimidin-2-yl]-4-piperidyl]acetate (43.0 mg, 73.9 mol, 1.0 eq) in DCM (1 mL) was added TFA (733 mg, 6.43 mmol, 480 μL, 87.1 eq). The mixture was stirred at 25° C. for 2 h. The mixture was concentrated to give a residue. Compound 2-[1-[5-[[3-(3-chloro-4-cyano-phenoxy)-2,2,4,4-tetramethyl-cyclobutyl]carbamoyl]pyrimidin-2-yl]-4-piperidyl]acetic acid (38 mg) was obtained as a white solid and directly used in the next step without further purification. LC-MS: MS (ESI⁺): t_{R}=0.999 min, m/z=526.2 [M+H⁺]

To a solution of 2-[1-[5-[[3-(3-chloro-4-cyano-phenoxy)-2,2,4,4-tetramethyl-cyclobutyl]carbamoyl]pyrimidin-2-yl]-4-piperidyl]acetic acid (38 mg), HATU (33 mg, 86 μmol, 1.2 equiv) in DMF (0.5 mL) was added 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (42 mg, 81 μmol, 1.1 eq) and DIPEA (28 mg, 0.22 mmol, 0.04 mL, 3.0 eq). The mixture was stirred at 25° C. for 2 h. The reaction was diluted with water (20 mL) and the resulting mixture was extracted with DCM (20 mL×2). The combined organic layers were dried over Na₂SO₄ and concentrated to give the residue. The residue was purified by semi-preparative reverse phase HPLC (column: Phenomenex luna C18 150*25 mm*10 um; mobile phase: [water (FA)-ACN]; gradient: 60%-90% B over 10 min). Compound 1-[1-[2-[1-[5-[[3-(3-chloro-4-cyano-phenoxy)-2,2,4,4-tetramethyl-cyclobutyl]carbamoyl]pyrimidin-2-yl]-4-piperidyl]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 101, 29 mg, 27 μmol, 37% yield over two steps) was obtained as a yellow solid. ¹H NMR (400 MHz, CDCl₃) δ=8.69 (s, 2H), 7.57 (d, J=8.8 Hz, 1H), 7.54 (s, 1H), 7.41 (s, 1H), 7.05 (s, 1H), 6.97 (d, J=2.4 Hz, 1H), 6.87 (s, 1H), 6.81 (dd, J=2.4, 8.8 Hz, 1H), 6.55 (t, J=55.6 Hz, 1H), 5.93 (d, J=8.3 Hz, 1H), 4.86 (br d, J=13.2 Hz, 2H), 4.76 (br d, J=13.2 Hz, 1H), 4.40 (q, J=4.4 Hz, 1H), 4.19-4.10 (m, 2H), 4.08-4.00 (m, 2H), 4.00-3.93 (m, 5H), 3.86-3.75 (m, 2H), 3.71 (q, J=5.2 Hz, 2H), 3.25-3.14 (m, 1H), 3.05-2.94 (m, 2H), 2.88 (br t, J=5.6 Hz, 2H), 2.79 (d, J=4.8 Hz, 4H), 2.75 (br t, J=5.2 Hz, 2H), 2.35-2.27 (m, 2H), 2.27-1.87 (m, 10H), 1.26 (s, 9H), 1.25 (br s, 6H), 1.22 (s, 6H). LC-MS: MS (ESI⁺): t_{R}=2.016 min, m/z=975.7 [M+H⁺]

### Example 3: Synthesis of 1-[1-[2-[[1-[5-[[3-(3-chloro-4-cyano-phenoxy)-2,2,4,4-tetramethyl-cyclobutyl]carbamoyl]pyrimidin-2-yl]-4-piperidyl]oxy]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 100)

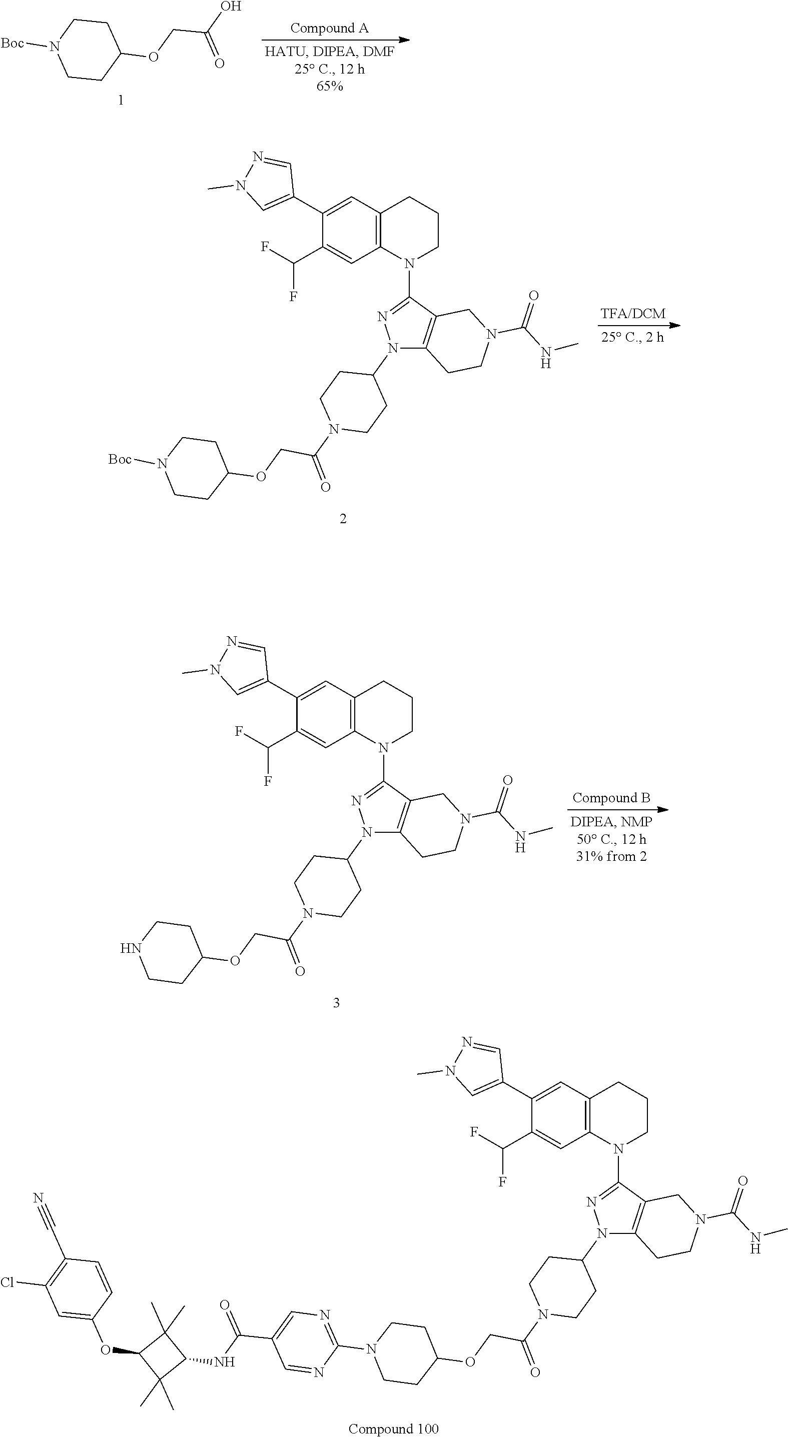 [SMILES: CC(C)(C)OC(=O)N1CCC(OCC(=O)O)CC1 ; CNC(=O)N1CCc2c(c(N3CCCc4cc(-c5cnn(C)c5)c(C(F)F)cc43)nn2C2CCN(C(=O)COC3CCN(C(=O)OC(C)(C)C)CC3)CC2)C1 ; CNC(=O)N1CCc2c(c(N3CCCc4cc(-c5cnn(C)c5)c(C(F)F)cc43)nn2C2CCN(C(=O)COC3CCNCC3)CC2)C1 ; CNC(=O)N1CCc2c(c(N3CCCc4cc(-c5cnn(C)c5)c(C(F)F)cc43)nn2C2CCN(C(=O)COC3CCN(c4ncc(C(=O)N[C@H]5C(C)(C)[C@H](Oc6ccc(C#N)c(Cl)c6)C5(C)C)cn4)CC3)CC2)C1]

To a solution of 2-[(1-tert-butoxycarbonyl-4-piperidyl)oxy]acetic acid (74.0 mg, 286 μmol, 1.5 eq) and 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (100 mg, 191 μmol, 1.0 eq) in DMF (1 mL) was added HATU (87 mg, 229 μmol, 1.2 eq) and DIPEA (74 mg, 572 μmol, 100 μL, 3.0 eq). The mixture was stirred at 25° C. for 12 h. The mixture was poured into ice-water (20 mL). The aqueous phase was extracted with dichloromethane (15 mL×3). The combined organic phase was washed with brine (10 mL×2), dried over anhydrous Na₂SO₄, filtered, and concentrated under vacuum. The residue was purified by prep-HPLC (column: Phenomenex luna C18 150*25 mm*10 um; mobile phase: [water(FA)-ACN]; gradient: 40%-70% B over 10 min) to give tert-butyl 4-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethoxy]piperidine-1-carboxylate (100 mg, 65% yield) was obtained as an off-white solid. LC-MS: MS (ESI⁺): t_{R}=0.570 min, m/z=766.6 [M+H⁺]

To a solution of tert-butyl 4-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethoxy]piperidine-1-carboxylate (100 mg, 131 μmol, 1.0 eq) in DCM (4 mL) was added TFA (1.5 g, 13.5 mmol, 1 mL, 103.1 eq). The mixture was stirred at 25° C. for 2 h. The mixture was concentrated to give a residue. Compound 3-[7-(difluoromethyl)-6-(1-methylpyrazol-4-yl)-3,4-dihydro-2H-quinolin-1-yl]-N-methyl-1-[1-[2-(4-piperidyloxy)acetyl]-4-piperidyl]-6,7-dihydro-4H-pyrazolo[4,3-c]pyridine-5-carboxamide (100 mg) was obtained as a brown solid and directly used in the next step without further purification. LC-MS: MS (ESI⁺): t_{R}=0.786 min, m/z=666.4 [M+H⁺]

To a solution of 3-[7-(difluoromethyl)-6-(1-methylpyrazol-4-yl)-3,4-dihydro-2H-quinolin-1-yl]-N-methyl-1-[1-[2-(4-piperidyloxy)acetyl]-4-piperidyl]-6,7-dihydro-4H-pyrazolo[4,3-c]pyridine-5-carboxamide (100 mg) and 2-chloro-N-[3-(3-chloro-4-cyano-phenoxy)-2,2,4,4-tetramethyl-cyclobutyl]pyrimidine-5-carboxamide (65 mg, 154 μmol, 1.2 eq) in NMP (1 mL) was added DIPEA (50 mg, 385 μmol, 67 μL, 3.0 eq). The mixture was stirred at 50° C. for 12 h. The reaction mixture was diluted with water (15 mL) and extracted with EtOAc (20 mL×3). The combined organic layers were concentrated to give a residue. The residue was purified by prep-HPLC (column: Phenomenex luna C18 150*25 mm*10 um; mobile phase: [water(FA)-ACN]; gradient: 30%-60% B over 10 min) to give 1-[1-[2-[[1-[5-[[3-(3-chloro-4-cyano-phenoxy)-2,2,4,4-tetramethyl-cyclobutyl]carbamoyl]pyrimidin-2-yl]-4-piperidyl]oxy]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 100, 43 mg, 31% yield over two steps). ¹H NMR (400 MHz, CDCl₃) δ=8.70 (s, 2H), 7.61-7.52 (m, 2H), 7.41 (s, 1H), 7.04 (s, 1H), 6.97 (d, J=2.4 Hz, 1H), 6.86 (s, 1H), 6.83-6.78 (m, 1H), 6.71-6.38 (m, 1H), 5.97 (d, J=8.4 Hz, 1H), 4.68 (d, J=12.8 Hz, 1H), 4.46-4.37 (m, 1H), 4.36-4.24 (m, 4H), 4.23-4.10 (m, 3H), 4.05 (s, 1H), 4.00-3.93 (m, 5H), 3.85-3.65 (m, 5H), 3.64-3.53 (m, 2H), 3.29-3.14 (m, 1H), 2.87 (m, 3H), 2.82-2.77 (m, 3H), 2.74 (m, 2H), 2.28-1.93 (m, 8H), 1.73-1.66 (m, 2H), 1.25 (s, 6H), 1.22 (s, 6H) LC-MS: MS (ESI⁺): t_{R}=2.706 min, m/z=1048.6 [M+H⁺]

### Example 4: Synthesis of 1-[1-[2-[2-[1-[5-[[3-(3-chloro-4-cyano-phenoxy)-2,2,4,4-tetramethyl-cyclobutyl]carbamoyl]pyrimidin-2-yl]-4-piperidyl]ethoxy]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 99)

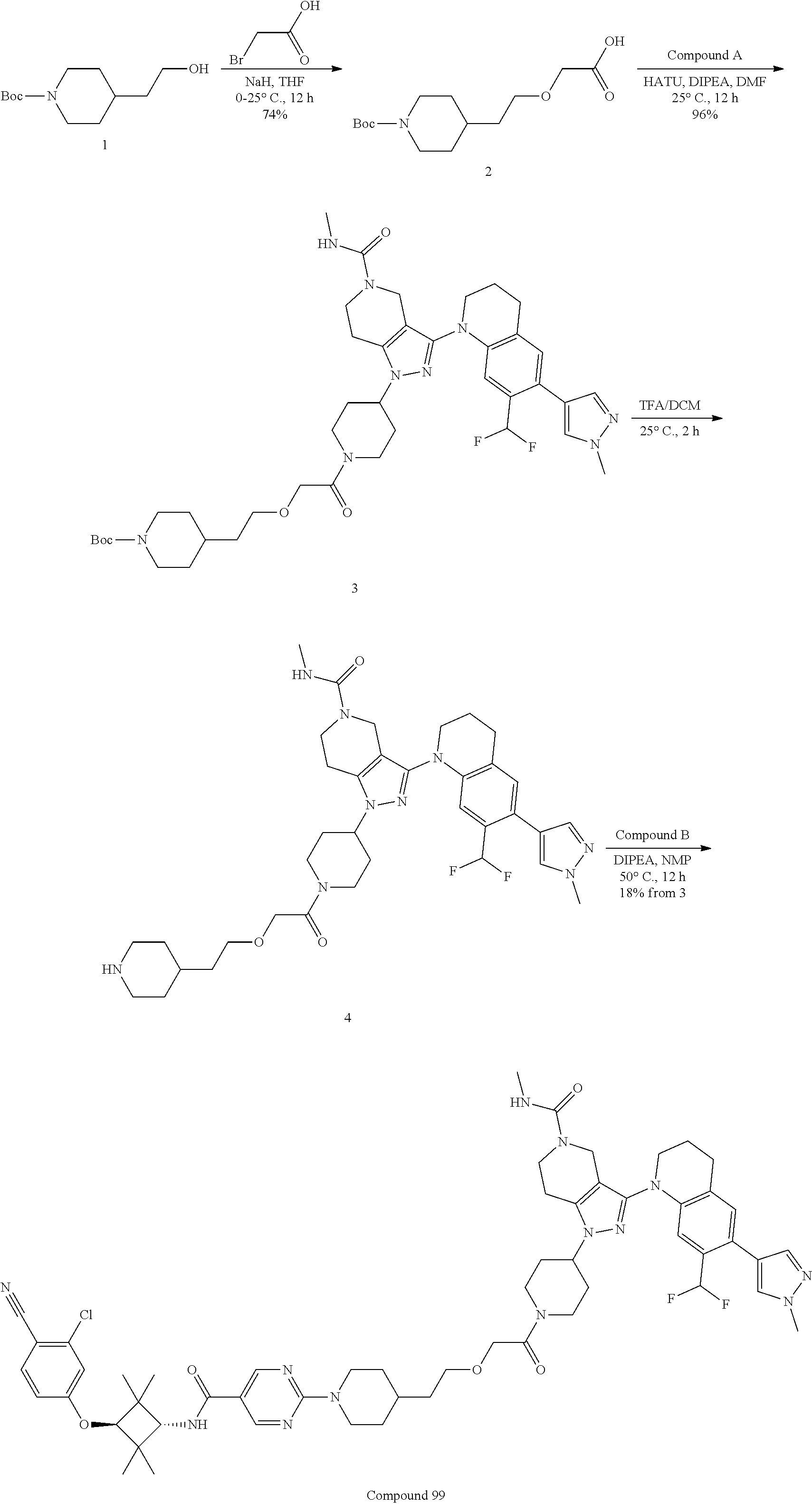 [SMILES: CC(C)(C)OC(=O)N1CCC(CCO)CC1 ; O=C(O)CBr ; CC(C)(C)OC(=O)N1CCC(CCOCC(=O)O)CC1 ; CNC(=O)N1CCc2c(c(N3CCCc4cc(-c5cnn(C)c5)c(C(F)F)cc43)nn2C2CCN(C(=O)COCCC3CCN(C(=O)OC(C)(C)C)CC3)CC2)C1 ; CNC(=O)N1CCc2c(c(N3CCCc4cc(-c5cnn(C)c5)c(C(F)F)cc43)nn2C2CCN(C(=O)COCCC3CCNCC3)CC2)C1 ; CNC(=O)N1CCc2c(c(N3CCCc4cc(-c5cnn(C)c5)c(C(F)F)cc43)nn2C2CCN(C(=O)COCCC3CCN(c4ncc(C(=O)N[C@H]5C(C)(C)[C@H](Oc6ccc(C#N)c(Cl)c6)C5(C)C)cn4)CC3)CC2)C1]

To a solution of NaH (349 mg, 60% w/w, 8.7 mmol, 3.0 eq) in THF (7 mL) was added tert-butyl 4-(2-hydroxyethyl)piperidine-1-carboxylate (2.0 g, 8.7 mmol, 3.0 eq) at 0° C. and stirred at 0° C. for 0.5 h. Then 2-bromoacetic acid (404 mg, 2.9 mmol, 209 μL, 1.0 eq) in THF (1 mL) was added dropwise at 0° C. The resulting mixture was stirred at 25° C. for 11.5 h. The reaction mixture was quenched with saturated aqueous NH₄Cl (2 mL) solution at 0° C. dropwise. And the resulting mixture was extracted with ethyl acetate (15 mL×3). The aqueous phase was adjusted to pH 6 with HCl (1 M) and then lyophilized to give a residue. The residue was used for the next step without further purification. Compound 2-[2-(1-tert-butoxycarbonyl-4-piperidyl)ethoxy]acetic acid (620 mg, 74% yield) was obtained as a yellow gum. LC-MS: MS (ES⁺): t_{R}=0.842 min, m/z=310.1 [M+Na]

To a solution of 2-[2-(1-tert-butoxycarbonyl-4-piperidyl)ethoxy]acetic acid (115 mg, 400 μmol, 3.0 eq) and HATU (61 mg, 160 μmol, 1.2 eq) in DMF (1 mL) was added DIPEA (52 mg, 400 μmol, 70 μL, 3.0 eq) and 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (70 mg, 133 μmol, 1.0 eq) in DMF (1 mL). The mixture was stirred at 25° C. for 12 h. The mixture was poured into ice-water (15 mL). The aqueous phase was extracted with dichloromethane (10 mL×3). The combined organic phase was washed with brine (10 mL×2), dried over anhydrous sodium sulfate, filtered, and concentrated under vacuum. The residue was purified by prep-TLC (silica, DCM/MeOH=15/1). Compound tert-butyl 4-[2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethoxy]ethyl]piperidine-1-carboxylate (120 mg, 128 μmol, 96% yield) was obtained as a yellow oil. LC-MS: MS (ESI⁺): t_{R}=0.970 min, m/z=794.4 [M+H⁺]

To a solution of tert-butyl 4-[2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethoxy]ethyl]piperidine-1-carboxylate (120 mg, 151 μmol, 1.0 eq) in DCM (1 mL) was added TFA (768 mg, 6.73 mmol, 0.5 mL, 44.5 eq). The mixture was stirred at 25° C. for 2 h. The mixture was concentrated to give a residue. Compound 3-[7-(difluoromethyl)-6-(1-methylpyrazol-4-yl)-3,4-dihydro-2H-quinolin-1-yl]-N-methyl-1-[1-[2-[2-(4-piperidyl)ethoxy]acetyl]-4-piperidyl]-6,7-dihydro-4H-pyrazolo[4,3-c]pyridine-5-carboxamide (100 mg) was obtained as a yellow oil and directly used in the next step without further purification. LC-MS: MS (ESI⁺): t_{R}=0.493 min, m/z=694.4 [M+H⁺]

To a solution of 3-[7-(difluoromethyl)-6-(1-methylpyrazol-4-yl)-3,4-dihydro-2H-quinolin-1-yl]-N-methyl-1-[1-[2-[2-(4-piperidyl)ethoxy]acetyl]-4-piperidyl]-6,7-dihydro-4H-pyrazolo[4,3-c]pyridine-5-carboxamide (100 mg) and 2-chloro-N-[3-(3-chloro-4-cyano-phenoxy)-2,2,4,4-tetramethyl-cyclobutyl]pyrimidine-5-carboxamide (78 mg, 186 μmol, 1.5 eq) in NMP (1 mL) was added DIPEA (48 mg, 371 μmol, 65 μL, 3.0 eq). The mixture was stirred at 50° C. for 12 h. After cooled to 25° C., the mixture was poured into ice-water (20 mL) and extracted with DCM (15 mL×3). The combined organic phase was washed with brine (10 mL×3), dried over anhydrous Na₂SO₄, filtered, and concentrated under vacuum. The residue was purified by semi-preparative reverse phase HPLC (column: Phenomenex luna C18 150*25 mm*10 um; mobile phase: [water(FA)-ACN]; gradient: 58%-88% B over 10 min). Compound 1-[1-[2-[2-[1-[5-[[3-(3-chloro-4-cyano-phenoxy)-2,2,4,4-tetramethyl-cyclobutyl]carbamoyl]pyrimidin-2-yl]-4-piperidyl]ethoxy]acetyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 99, 25 mg, 18% yield over two steps) was obtained as a yellow solid. ¹H NMR (400 MHz, CHLOROFORM-d) δ=8.67 (s, 2H), 7.62-7.51 (m, 2H), 7.42 (s, 1H), 7.06-7.00 (m, 1H), 6.97 (d, J=2.4 Hz, 1H), 6.85 (s, 1H), 6.83-6.79 (m, 1H), 6.70-6.39 (m, 1H), 5.92 (d, J=7.6 Hz, 1H), 4.88-4.77 (m, 2H), 4.68 (s, 1H), 4.40 (m, 1H), 4.23-4.09 (m, 5H), 4.05 (s, 1H), 3.96 (s, 5H), 3.86-3.75 (m, 2H), 3.73-3.66 (m, 2H), 3.61 (m, 2H), 3.20 (s, 1H), 3.00-2.90 (m, 2H), 2.85 (m, 3H), 2.81-2.69 (m, 6H), 2.24-1.92 (m, 6H), 1.88-1.72 (m, 3H), 1.64-1.61 (m, 3H), 1.25 (s, 6H), 1.22 (s, 6H). LC-MS: MS (ESI⁺): t_{R}=2.55 min, m/z=1076.7 [M+H⁺]

### Example 5: Synthesis of 2-[4-[2-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-oxo-ethyl]piperazin-1-yl]-N-[3-(3-chloro-4-cyano-phenoxy)-2,2,4,4-tetramethyl-cyclobutyl]pyrimidine-5-carboxamide (Compound 96)

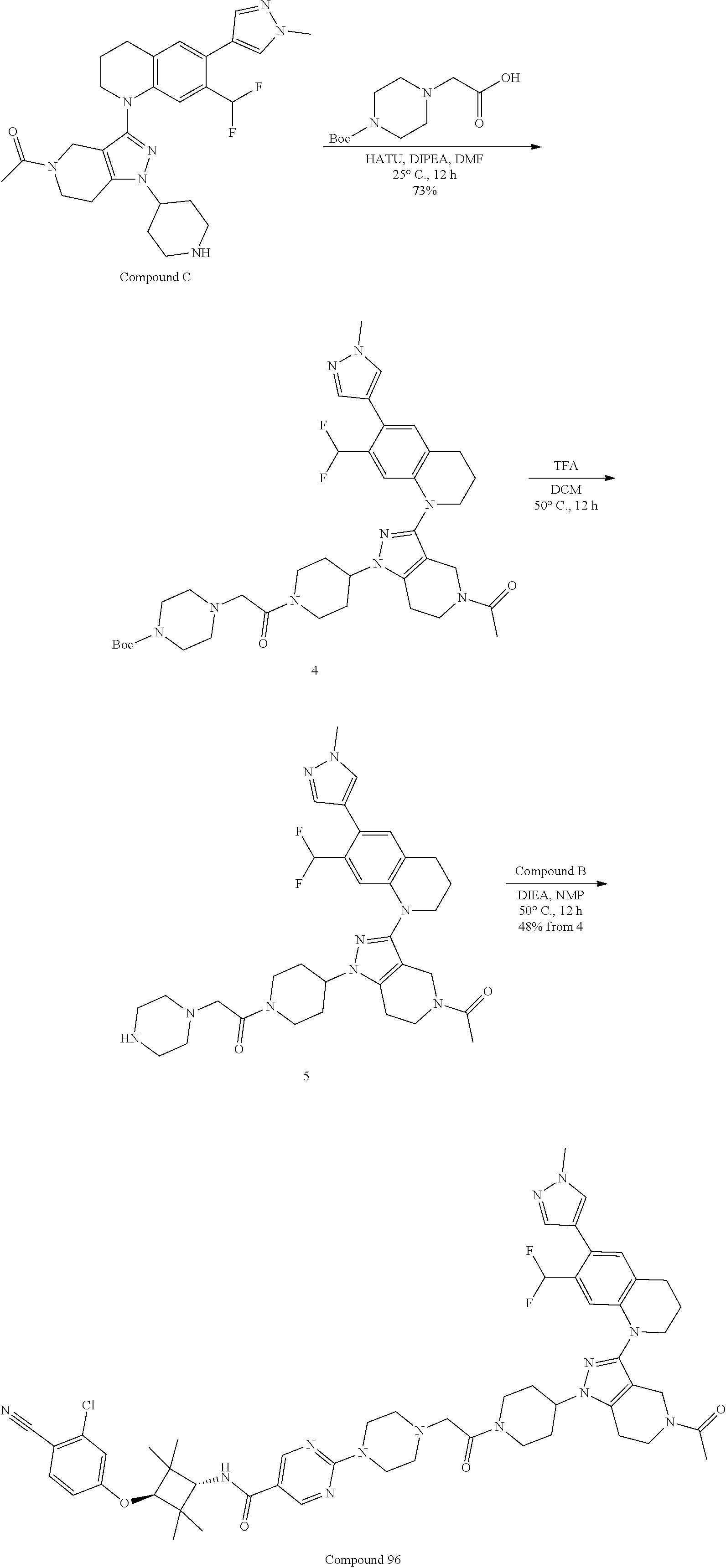 [SMILES: CC(=O)N1CCc2c(c(N3CCCc4cc(-c5cnn(C)c5)c(C(F)F)cc43)nn2C2CCNCC2)C1 ; CC(C)(C)OC(=O)N1CCN(CC(=O)O)CC1 ; CC(=O)N1CCc2c(c(N3CCCc4cc(-c5cnn(C)c5)c(C(F)F)cc43)nn2C2CCN(C(=O)CN3CCN(C(=O)OC(C)(C)C)CC3)CC2)C1 ; CC(=O)N1CCc2c(c(N3CCCc4cc(-c5cnn(C)c5)c(C(F)F)cc43)nn2C2CCN(C(=O)CN3CCNCC3)CC2)C1 ; CC(=O)N1CCc2c(c(N3CCCc4cc(-c5cnn(C)c5)c(C(F)F)cc43)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1]

To a solution of 2-(4-tert-butoxycarbonylpiperazin-1-yl)acetic acid (71.9 mg, 294 μmol, 1.5 eq) in DMF (1 mL) was added HATU (119 mg, 314 μmol, 1.6 eq) and stirred at 25° C. for 0.5 h. 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (100 mg, 196 μmol, 1.0 eq) and Et₃N (59.6 mg, 589 μmol, 81.9 μL, 3.0 eq) was added to the above reaction mixture and stirred at 25° C. for 11.5 h. The mixture was poured into ice-water (15 mL). The aqueous phase was extracted with dichloromethane (10 mL×3). The combined organic phase was washed with brine (10 mL×2), dried over anhydrous sodium sulfate, filtered and concentrated under vacuum. The residue was purified by semi-preparative reverse phase HPLC (column: Phenomenex luna C18 150*25 mm*10 um; mobile phase: [water(FA)-ACN]; gradient: 18%-48% B over 10 min). Compound tert-butyl 4-[2-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-oxo-ethyl]piperazine-1-carboxylate (105 mg, 143 μmol, 73% yield) was obtained as a white solid. LC-MS: MS (ESI⁺): t_{R}=0.521 min, m/z=736.6 [M+H⁺]

To a solution of tert-butyl 4-[2-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-oxo-ethyl]piperazine-1-carboxylate (105 mg, 143 mol, 1 eq) in DCM (1 mL) was added TFA (2.69 g, 23.6 mmol, 1.75 mL, 165 eq) and stirred at 25° C. for 1 h. The mixture was concentrated to give a residue. Compound 1-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-piperazin-1-yl-ethanone (0.12 g) was obtained as a brown solid and directly used in the next step without further purification. LC-MS: MS (ESI⁺): t_{R}=0.474 min, m/z=636.5 [M+H⁺]

To a solution of 1-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-piperazin-1-yl-ethanone (0.12 g) in NMP (1 mL) was added DIEA (103 mg, 800 μmol, 139 μL, 5.0 eq) and 2-chloro-N-[3-(3-chloro-4-cyano-phenoxy)-2,2,4,4-tetramethyl-cyclobutyl]pyrimidine-5-carboxamide (67.1 mg, 160 μmol, 1.0 eq). The reaction mixture was stirred at 50° C. for 12 h. After cooled to 25° C., the mixture was poured into ice-water (20 mL) and extracted with DCM (15 mL×3). The combined organic phase was washed with brine (10 mL×3), dried over anhydrous sodium sulfate, filtered and concentrated under vacuum. The residue was purified by semi-preparative reverse phase HPLC (column: Phenomenex luna C18 150*25 mm*10 um; mobile phase: [water(FA)-ACN]; gradient: 28%-58% B over 10 min). Compound 2-[4-[2-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-oxo-ethyl]piperazin-1-yl]-N-[3-(3-chloro-4-cyano-phenoxy)-2,2,4,4-tetramethyl-cyclobutyl]pyrimidine-5-carboxamide (Compound 96, 81.04 mg, 77.29 μmol, 48% yield over two steps) was obtained as a yellow solid. ¹H NMR (400 MHz, CDCl₃): δ=8.69 (s, 2H) 7.57 (d, J=8.8 Hz, 1H) 7.48-7.54 (m, 1H) 7.35-7.42 (m, 1H) 7.27 (s, 1H) 6.98-7.06 (m, 1H) 6.96 (d, J=2.4 Hz, 1H) 6.85-6.91 (m, 1H) 6.80 (m, 1H) 6.32-6.68 (m, 1H) 6.01 (d, J=8.4 Hz, 1H) 4.62-4.77 (m, 1H) 4.19-4.34 (m, 2H) 4.10-4.18 (m, 3H) 4.05 (s, 1H) 3.94 (m, 6H) 3.83-3.92 (m, 1H) 3.64-3.80 (m, 4H) 3.16-3.38 (m, 3H) 2.72-2.92 (m, 5H) 2.61 (m, 4H) 2.20-2.34 (m, 1H) 2.17 (s, 1H) 1.97-2.08 (m, 6H) 1.25 (s, 6H) 1.20-1.23 (m, 6H). LC-MS: MS (ESI⁺): t_{R}=1.97 min, m/z=1018.7 [M+H⁺]

### Example 6: Synthesis of 2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]-N-[3-(3-chloro-4-cyano-phenoxy)-2,2,4,4-tetramethyl-cyclobutyl]pyrimidine-5-carboxamide (Compound 85)

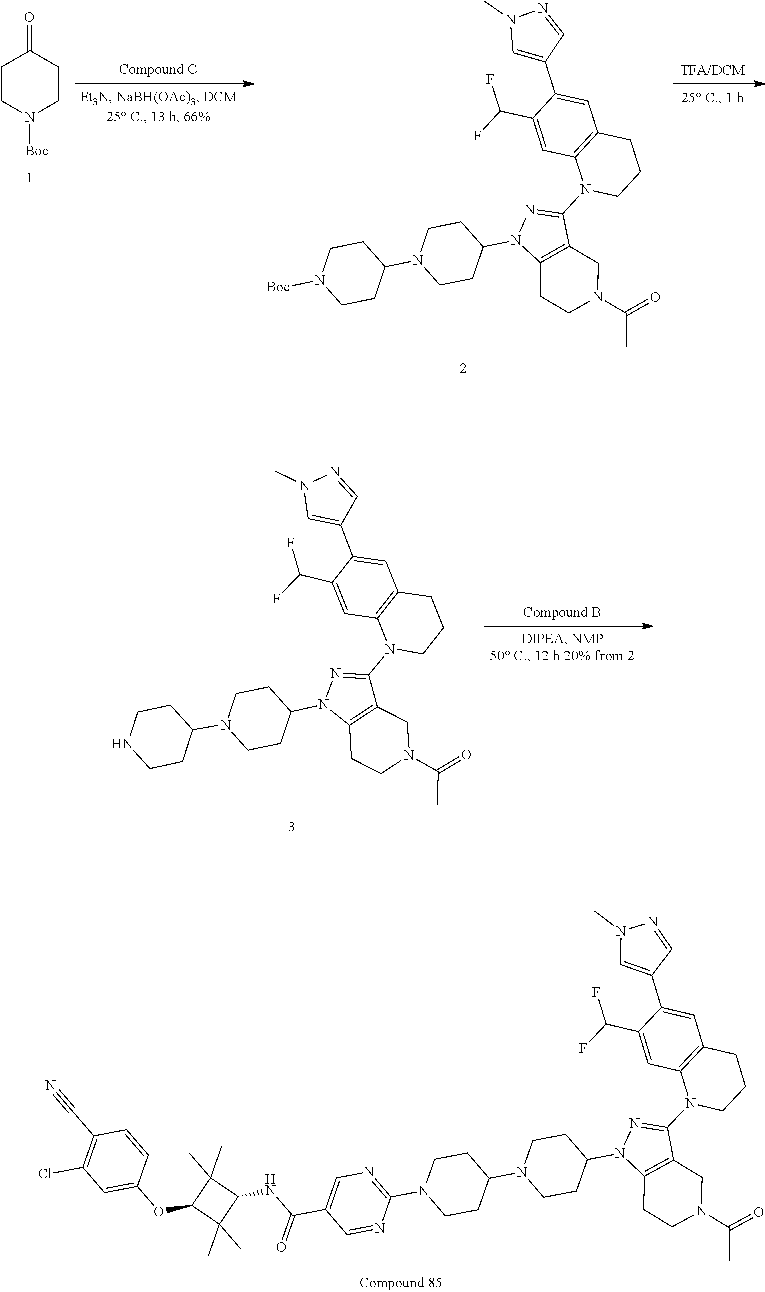 [SMILES: CC(C)(C)OC(=O)N1CCC(=O)CC1 ; CC(=O)N1CCc2c(c(N3CCCc4cc(-c5cnn(C)c5)c(C(F)F)cc43)nn2C2CCN(C3CCN(C(=O)OC(C)(C)C)CC3)CC2)C1 ; CC(=O)N1CCc2c(c(N3CCCc4cc(-c5cnn(C)c5)c(C(F)F)cc43)nn2C2CCN(C3CCNCC3)CC2)C1 ; CC(=O)N1CCc2c(c(N3CCCc4cc(-c5cnn(C)c5)c(C(F)F)cc43)nn2C2CCN(C3CCN(c4ncc(C(=O)N[C@H]5C(C)(C)[C@H](Oc6ccc(C#N)c(Cl)c6)C5(C)C)cn4)CC3)CC2)C1]

To a solution of tert-butyl 4-oxopiperidine-1-carboxylate (235 mg, 1.18 mmol, 4.0 eq), 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (150 mg, 294 μmol, 1.0 eq) in DCM (3 mL) was added Et₃N (109 mg, 1.0 mmol, 0.15 mL, 3.7 equiv) at 25° C. for 1 h. Then NaBH(OAc)₃ (312 mg, 1.47 mmol, 5.0 eq) was added. The reaction mixture was stirred at 25° C. for 12 h. The reaction was diluted with water (10 mL) and the resulting mixture was extracted with methylene chloride (20 mL×2). The combined organic layers were dried over Na₂SO₄ and concentrated to give the residue. The residue was purified by prep-TLC (silica, DCM/MeOH=10/1). Compound tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]piperidine-1-carboxylate (138 mg, 195 μmol, 66% yield) was obtained as a yellow oil. LC-MS: MS (ESI⁺): t_{R}=0.813 min, m/z=693.5 [M+H⁺]

To a solution of tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]piperidine-1-carboxylate (80.0 mg, 115 μmol, 1.0 eq) in DCM (1 mL) was added TFA (768 mg, 6.73 mmol, 0.5 mL, 58 eq). The mixture was stirred at 25° C. for 2 h. The mixture was concentrated to give a residue. Compound 1-[3-[7-(difluoromethyl)-6-(1-methylpyrazol-4-yl)-3,4-dihydro-2H-quinolin-1-yl]-1-[1-(4-piperidyl)-4-piperidyl]-6,7-dihydro-4H-pyrazolo[4,3-c]pyridin-5-yl]ethanone (81 mg) was obtained as a yellow oil and directly used in the next step without further purification. LC-MS: MS (ESI⁺): t_{R}=0.457 min, m/z=593.5 [M+H⁺]

To a solution of 1-[3-[7-(difluoromethyl)-6-(1-methylpyrazol-4-yl)-3,4-dihydro-2H-quinolin-1-yl]-1-[1-(4-piperidyl)-4-piperidyl]-6,7-dihydro-4H-pyrazolo[4,3-c]pyridin-5-yl]ethanone (81 mg) in NMP (0.5 mL) was added 2-chloro-N-[3-(3-chloro-4-cyano-phenoxy)-2,2,4,4-tetramethyl-cyclobutyl]pyrimidine-5-carboxamide (48 mg, 115 μmol, 1.0 eq) and DIPEA (45.0 mg, 344 μmol, 60.0 uL, 3.0 eq). The mixture was stirred at 50° C. for 12 h. The reaction was diluted with water (20 mL) and the resulting mixture was extracted with DCM (20 mL×2). The combined organic layers were dried over Na₂SO₄ and concentrated to give the residue. The residue was purified by semi-preparative reverse phase HPLC (column: Phenomenex luna C18 150*25 mm*10 um; mobile phase: [water(FA)-ACN]; gradient: 28%-58% B over 10 min). Compound 2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]-N-[3-(3-chloro-4-cyano-phenoxy)-2,2,4,4-tetramethyl-cyclobutyl]pyrimidine-5-carboxamide (Compound 85, 23.2 mg, 23.7 μmol, 20% yield over two steps) was obtained as an off-white solid. ¹H NMR (400 MHz, DMSO-d₆) δ=8.79 (s, 2H), 8.13 (s, 1H), 7.90 (d, J=8.8 Hz, 1H), 7.79-7.71 (m, 2H), 7.50 (s, 1H), 7.21 (d, J=2.4 Hz, 1H), 7.11 (s, 1H), 7.01 (dd, J=2.4, 8.8 Hz, 1H), 6.80 (br s, 2H), 4.95-4.75 (m, 2H), 4.30 (s, 1H), 4.20-4.14 (m, 1H), 4.11 (br s, 1H), 4.07-4.02 (m, 1H), 3.86 (s, 3H), 3.77-3.67 (m, 2H), 3.63-3.55 (m, 2H), 3.30-3.09 (m, 3H), 3.06-2.92 (m, 3H), 2.90-2.79 (m, 4H), 2.77-2.70 (m, 1H), 2.20-2.03 (m, 5H), 2.02-1.88 (m, 5H), 1.65-1.42 (m, 2H), 1.30-1.24 (m, 1H), 1.22 (s, 6H), 1.11 (s, 6H). LC-MS: MS (ESI⁺): t_{R}=2.016 min, m/z=975.7 [M+H⁺]

The compounds below were prepared in a similar manner as described in Example 6.

| | | | LC-MS |
|---|---|---|---|
| Compound | Name | ¹H NMR (CDCl₃) | [M + H⁺]/2 |
| 276 | 2-[9-[5-acetyl-3-[7- | δ = 8.71 (s, J = 9.6 Hz, 2H), 7.57 (d, | 480.9 |
| | (difluoromethyl)-6-(1- | J = 8.8 Hz, 1 H), 7.54-7.53 (m, 1H), | |
| | methylpyrazol-4-yl)-3,4- | 7.42-7.41 (m, 1H), 7.10 (s, 1H), 6.97-6.96 | |
| | dihydro-2H-quinolin-1-yl]-6,7- | (m, 1H), 6.91-6.90 (m, 1H), 6.81 (dd, | |
| | dihydro-4H-pyrazolo[4,3- | 1H, J = 2.4, 8.8 Hz), 6.54-6.40 (m, 1H), | |
| | c]pyridin-1-yl]-3- | 5.92 (d, J = 8 Hz, 1 H), 4.28-4.14 (m, | |
| | azaspiro[5.5]undecan-3-yl]-N- | 3H), 4.13 (s, 1H), 3.96 (s, 3H), 3.95-3.90 | |
| | [3-(3-chloro-4-cyano-phenoxy)- | (m, 6H), 3.74-3.73 (m, 3H), 2.89-2.88 | |
| | 2,2,4,4-tetramethyl- | (m, 2H), 2.85-2.83 (m, 1H), 2.75-2.73 | |
| | cyclobutyl]pyrimidine-5- | (m, 1H), 2.25-2.23 (m, 1H), 2.22-2.20 | |
| | carboxamide | (m, 2H), 2.19-2.18(m, 1 H), 2.15-2.13 | |
| | | (m, 3H), 1.96-1.94 (m, 2H), 1.80-1.75(m, | |
| | | 2H), 1.75-1.73 (m, 2H), 1.49-1.48 | |
| | | (m, 2H), 1.30-1.25 (m, 2H), 1.25 | |
| | | (s, 6H), 1.22 (s, 6H) | |

### Example 7: Synthesis of 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]-N-[3-(3-chloro-4-cyano-phenoxy)-2,2,4,4-tetramethyl-cyclobutyl]pyrimidine-5-carboxamide (Compound 84)

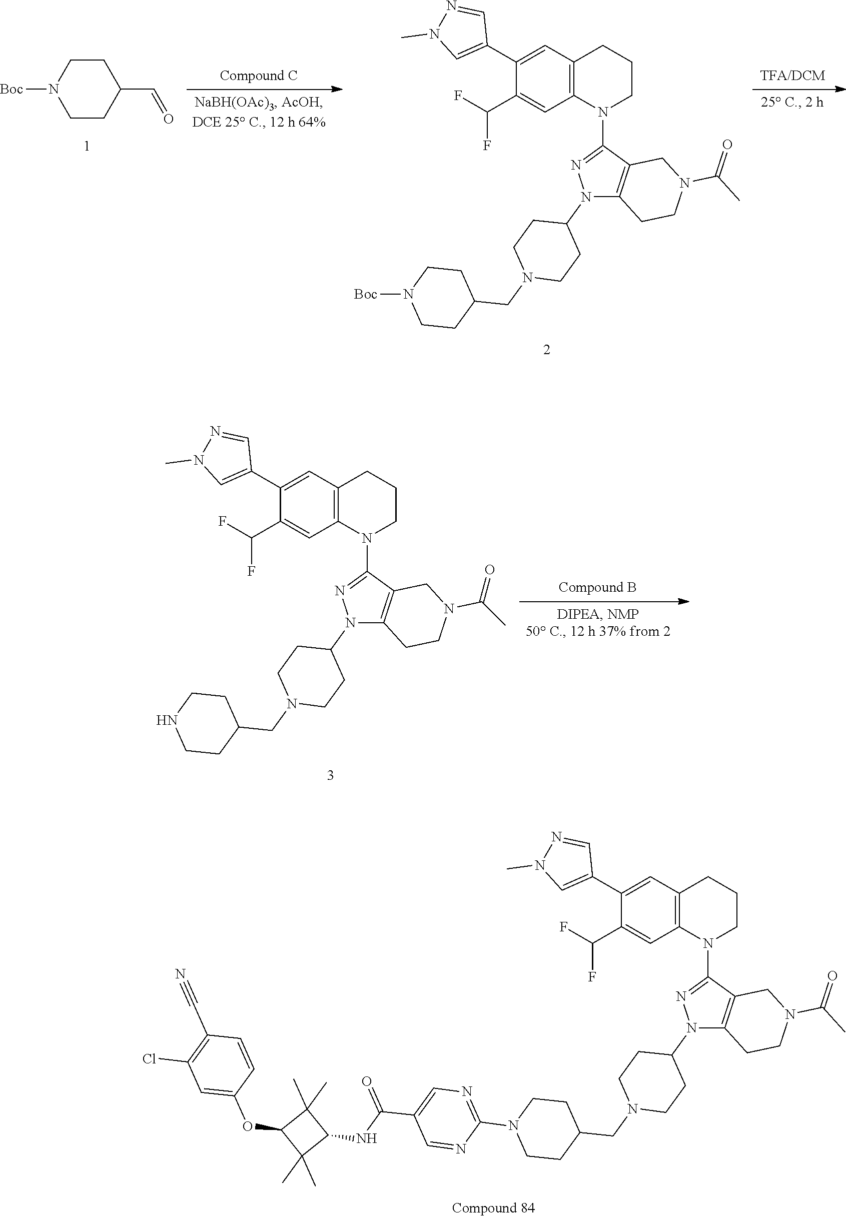 [SMILES: CC(C)(C)OC(=O)N1CCC(C=O)CC1 ; CC(=O)N1CCc2c(c(N3CCCc4cc(-c5cnn(C)c5)c(C(F)F)cc43)nn2C2CCN(CC3CCN(C(=O)OC(C)(C)C)CC3)CC2)C1 ; CC(=O)N1CCc2c(c(N3CCCc4cc(-c5cnn(C)c5)c(C(F)F)cc43)nn2C2CCN(CC3CCNCC3)CC2)C1 ; CC(=O)N1CCc2c(c(N3CCCc4cc(-c5cnn(C)c5)c(C(F)F)cc43)nn2C2CCN(CC3CCN(c4ncc(C(=O)N[C@H]5C(C)(C)[C@H](Oc6ccc(C#N)c(Cl)c6)C5(C)C)cn4)CC3)CC2)C1]

To a solution of tert-butyl 4-formylpiperidine-1-carboxylate (63 mg, 294 μmol, 1.5 eq) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (100 mg, 196 μmol, 1.0 eq) in DCM (3 mL) was added AcOH (18 mg, 294 μmol, 17 μL, 1.5 eq) and NaBH(OAc)₃ (125 mg, 589 μmol, 3.0 eq). The mixture was stirred at 25° C. for 12 h. The mixture was poured into ice-water (20 mL). The aqueous phase was extracted with DCM (15 mL×3). The combined organic phase was washed with brine (10 mL×2), dried over anhydrous Na₂SO₄, filtered, and concentrated under vacuum. The residue was purified by prep-HPLC (column: Phenomenex luna C18 150*25 mm*10 um; mobile phase: [water(FA)-ACN]; gradient: 20%-50% B over 10 min) to give tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]piperidine-1-carboxylate (90 mg, 125 μmol, 64% yield) was obtained as an off-white solid. LC-MS: MS (ESI⁺): t_{R}=0.529 min, m/z=707.5 [M+H⁺]

To a solution of tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]piperidine-1-carboxylate (90 mg, 127 mol, 1.0 eq) in DCM (1 mL) was added TFA (921 mg, 8.1 mmol, 600 μL, 63.4 eq). The mixture was stirred at 25° C. for 2 h. The mixture was concentrated to give a residue. Compound 1-[3-[7-(difluoromethyl)-6-(1-methylpyrazol-4-yl)-3,4-dihydro-2H-quinolin-1-yl]-1-[1-(4-piperidylmethyl)-4-piperidyl]-6,7-dihydro-4H-pyrazolo[4,3-c]pyridin-5-yl]ethanone (70 mg) was obtained as a brown solid and directly used in the next step without further purification. LC-MS: MS (ESI⁺): t_{R}=0.747 min, m/z=607.5 [M+H⁺]

To a solution of 1-[3-[7-(difluoromethyl)-6-(1-methylpyrazol-4-yl)-3,4-dihydro-2H-quinolin-1-yl]-1-[1-(4-piperidylmethyl)-4-piperidyl]-6,7-dihydro-4H-pyrazolo[4,3-c]pyridin-5-yl]ethanone (70 mg) and 2-chloro-N-[3-(3-chloro-4-cyano-phenoxy)-2,2,4,4-tetramethyl-cyclobutyl]pyrimidine-5-carboxamide (58 mg, 138 μmol, 1.2 eq) in NMP (1 mL) was added DIPEA (45 mg, 346 μmol, 60 μL, 3.0 eq). The mixture was stirred at 50° C. for 12 h. The reaction mixture was diluted with water (15 mL) and extracted with EtOAc (20 mL×3). The combined organic layers were concentrated to give a residue. The residue was purified by prep-HPLC (column: Phenomenex luna C18 150*25 mm*10 um; mobile phase: [water(FA)-ACN]; gradient: 30%-60% B over 10 min) to give 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]-N-[3-(3-chloro-4-cyano-phenoxy)-2,2,4,4-tetramethyl-cyclobutyl]pyrimidine-5-carboxamide (Compound 84, 43 mg, 43 μmol, 37% yield over two steps). ¹H NMR (400 MHz, CDCl₃) δ=8.70 (s, 2H), 7.60-7.50 (m, 2H), 7.44-7.35 (m, 1H), 7.09-6.94 (m, 2H), 6.91-6.85 (m, 1H), 6.83-6.77 (m, 1H), 6.72-6.34 (m, 1H), 5.93 (d, J=8.4 Hz, 1H), 4.85 (m, 2H), 4.73 (s, 4H), 4.26 (s, 1H), 4.17-4.07 (m, 2H), 4.04 (s, 1H), 3.99-3.86 (m, 5H), 3.78-3.66 (m, 3H), 3.12-2.70 (m, 8H), 2.33-2.23 (m, 4H), 2.21-2.13 (m, 3H), 2.10-2.01 (m, 4H), 1.99-1.95 (m, 1H), 1.25 (s, 6H), 1.22 (s, 6H), 1.20-1.10 (m, 2H). LC-MS: MS (ESI⁺): t_{R}=2.052 min, m/z=989.6 [M+H⁺]

The compounds below were prepared in a similar manner as described in Example 7.

| | | | LC-MS |
|---|---|---|---|
| Compound | Name | ¹H NMR (CDCl₃) | [M + H⁺] |
| 264 | 4-[4-[[4-[5-acetyl-3-[7- | δ = 7.69 (d, J = 8.8 Hz, 2H), 7.53 (d, J = | 1013.6 |
| | (difluoromethyl)-6-(1- | 7.2 Hz, 1H), 7.45 (d, J = 8.4 Hz, 1H), | |
| | methylpyrazol-4-yl)-3,4- | 7.40 (d, J = 6.8 Hz, 1H), 7.08-6.97 (m, | |
| | dihydro-2H-quinolin-1-yl]-6,7- | 1H), 6.97-6.82 (m, 3H), 6.69-6.38 (m, | |
| | dihydro-4H-pyrazolo[4,3- | 3H), 6.15 (br d, J = 8.0 Hz, 1H), 4.27 (s, | |
| | c]pyridin-1-yl]-1- | 1H), 4.18-4.11 (m, 2H), 4.05 (s, 1H), | |
| | piperidyl]methyl]-4-methoxy-1- | 3.95 (d, J = 2.0 Hz, 3H), 3.92 (s, 3H), | |
| | piperidyl]-N-[3-(4-cyano-3- | 3.91-3.87 (m, 1H), 3.78-3.65 (m, 3H), | |
| | methoxy-phenoxy)-2,2,4,4- | 3.51 (br d, J = 12.4 Hz, 2H), 3.26 (s, 3H), | |
| | tetramethyl- | 3.22-3.11 (m, 3H), 2.91-2.78 (m, 3H), | |
| | cyclobutyl]benzamide | 2.74 (br t, J = 5.6 Hz, 1H), 2.69-2.49 | |
| | | (m, 3H), 2.34-2.24 (m, 2H), 2.17 (s, | |
| | | 2H), 2.11-2.00 (m, 5H), 1.94 (br d, J = | |
| | | 12.8 Hz, 5H), 1.77-1.69 (m, 2H), 1.25 | |
| | | (d, J = 12.4 Hz, 12H) | |

### Example 8: Synthesis of 1-[1-[[1-[5-[[3-(3-chloro-4-cyano-phenoxy)-2,2,4,4-tetramethyl-cyclobutyl]carbamoyl]pyrimidin-2-yl]-4-piperidyl]methyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 82)

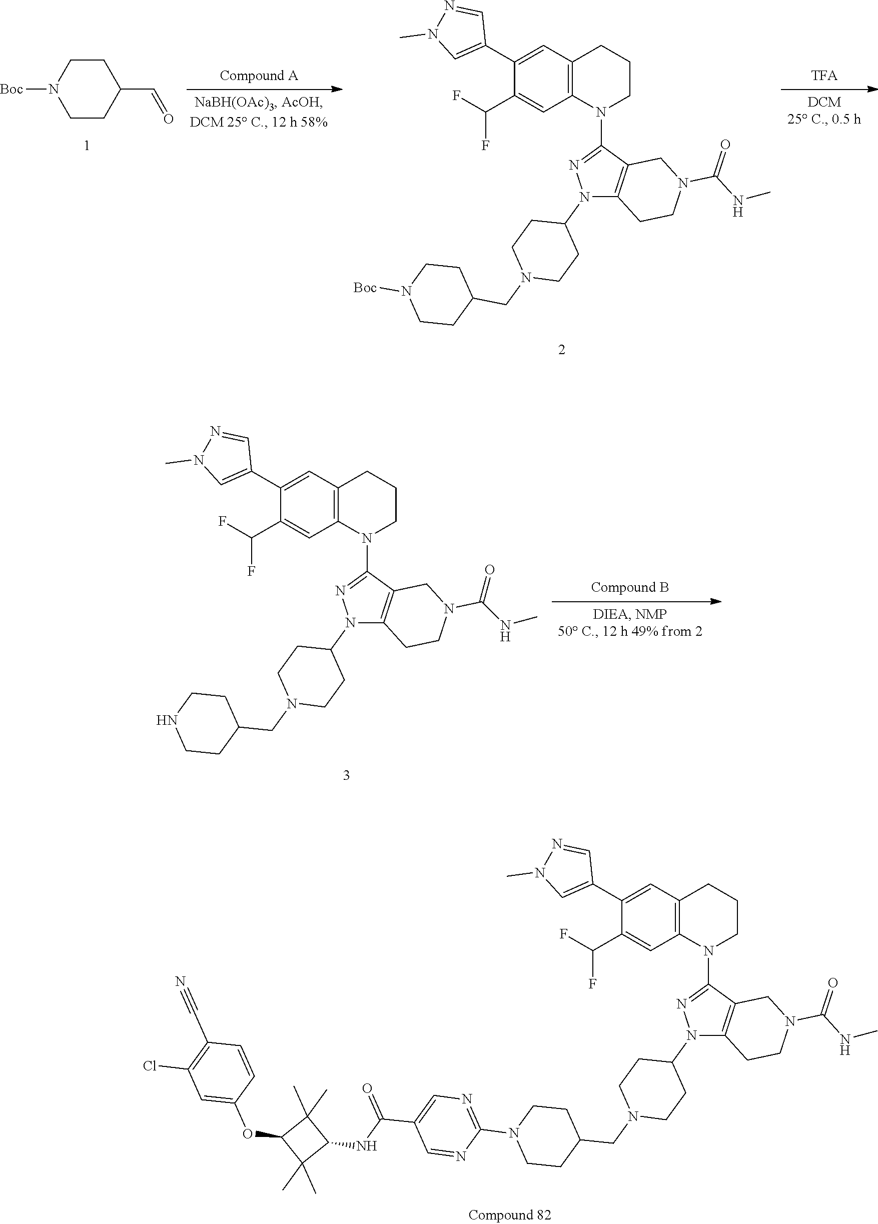

To a solution of 3-[7-(difluoromethyl)-6-(1-methylpyrazol-4-yl)-3,4-dihydro-2H-quinolin-1-yl]-N methyl-1-(4-piperidyl)-6,7-dihydro-4H-pyrazolo[4,3-c]pyridine-5-carboxamide (80.0 mg, 153 μmol, 1.0 eq) and tert-butyl 4-formylpiperidine-1-carboxylate (65.1 mg, 305.0 mol, 2.0 eq) in DCM (4 mL) was added AcOH (18.3 mg, 305 μmol, 17.5 μL, 2.0 eq) and stirred at 25° C. for 0.5 h. NaBH(OAc)₃ (129 mg, 610 μmol, 4.0 eq) was added to the above reaction mixture. The mixture was stirred at 25° C. for 12 h. The reaction mixture was quenched by addition of saturated aqueous NaHCO₃ (30 mL) at 0° C., and then extracted with DCM/MeOH (10:1, 30 mL×3). The combined organics were washed with H₂O (50 mL×2), dried over anhydrous Na₂SO₄, filtered, and concentrated under reduced pressure to give a residue. The residue was purified by prep-HPLC (column: Phenomenex luna C18 150*25 mm*10 um; mobile phase: [water(FA)-ACN]; gradient: 20%-50% B over 10 min). Compound tert-butyl 4-[[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]methyl]piperidine-1-carboxylate (64.0 mg, 88.7 μmol, 58% yield) was obtained as a white solid. LC-MS: MS (ESI⁺): t_{R}=0.795 min, m/z=722.4 [M+H⁺]

To a solution of tert-butyl 4-[[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]methyl]piperidine-1-carboxylate (79.0 mg, 109 μmol, 1.0 eq) in DCM (2 mL) was added TFA (1 mL). The mixture was stirred at 25° C. for 0.5 h. The reaction mixture was concentrated in vacuum. The residue was used for the next step without further purification. Compound 3-[7-(difluoromethyl)-6-(1-methylpyrazol-4-yl)-3,4-dihydro-2H-quinolin-1-yl]-N-methyl-1-[1-(4-piperidylmethyl)-4-piperidyl]-6,7-dihydro-4H-pyrazolo[4,3-c]pyridine-5-carboxamide (80 mg) was obtained as a yellow oil. LC-MS: MS (ESI⁺): t_{R}=0.687 min, m/z=622.4 [M+H⁺]

To a solution of 3-[7-(difluoromethyl)-6-(1-methylpyrazol-4-yl)-3,4-dihydro-2H-quinolin-1-yl]-N-methyl-1-[1-(4-piperidylmethyl)-4-piperidyl]-6,7-dihydro-4H-pyrazolo[4,3-c]pyridine-5-carboxamide (80 mg) and 2-chloro-N-[3-(3-chloro-4-cyano-phenoxy)-2,2,4,4-tetramethyl-cyclobutyl]pyrimidine-5-carboxamide (54.7 mg, 131 μmol, 1.2 eq) in NMP (0.5 mL) was added DIPEA (0.1 mL). The mixture was stirred at 50° C. for 12 h. The reaction mixture was filtered to afford crude product. The residue was then purified by prep-HPLC (column: Phenomenex luna C18 150*25 mm*10 um; mobile phase: [water(FA)-ACN]; gradient: 30%-60% B over 10 min). Compound 1-[1-[[1-[5-[[3-(3-chloro-4-cyano-phenoxy)-2,2,4,4-tetramethyl-cyclobutyl]carbamoyl]pyrimidin-2-yl]-4-piperidyl]methyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 82, 54.4 mg, 53.6 mol, 49% yield over two steps) was obtained as a yellow solid.

¹H NMR (400 MHz, CDCl₃): δ=8.70 (s, 2H), 8.38-8.36 (m, 1H), 7.62-7.51 (m, 2H), 7.41 (s, 1H), 7.04 (s, 1H), 6.98-6.95 (m, 1H), 6.87 (s, 1H), 6.83-6.78 (m, 1H), 6.55 (m, J=55.6 Hz, 1H), 5.98-5.91 (m, 1H), 4.85 (m, J=13.2 Hz, 2H), 4.46-4.39 (m, 1H), 4.13 (m, J=8.1 Hz, 1H), 4.04 (s, 1H), 3.98 (s, 2H), 3.96 (s, 3H), 3.78 (m, J=5.6 Hz, 2H), 3.75-3.70 (m, 2H), 3.12-3.04 (m, 2H), 2.95 (m, J=11.9 Hz, 2H), 2.87 (m, J=6.2 Hz, 2H), 2.80-2.77 (m, 3H), 2.75 (m, J=5.4 Hz, 2H), 2.34-2.28 (m, 3H), 2.20 (m, 1H), 2.10-2.03 (m, 3H), 2.03-1.90 (m, 7H), 1.23 (m, J=13.7 Hz, 11H), 1.20-1.11 (m, 2H) LC-MS: MS (ESI⁺): t_{R}=2.000 min, m/z=1004.6 [M+H⁺]

### Example 9: Synthesis of [1-[5-[[3-(3-chloro-4-cyano-phenoxy)-2,2,4,4-tetramethyl-cyclobutyl]carbamoyl]pyrimidin-2-yl]-4-piperidyl]4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (Compound 70)

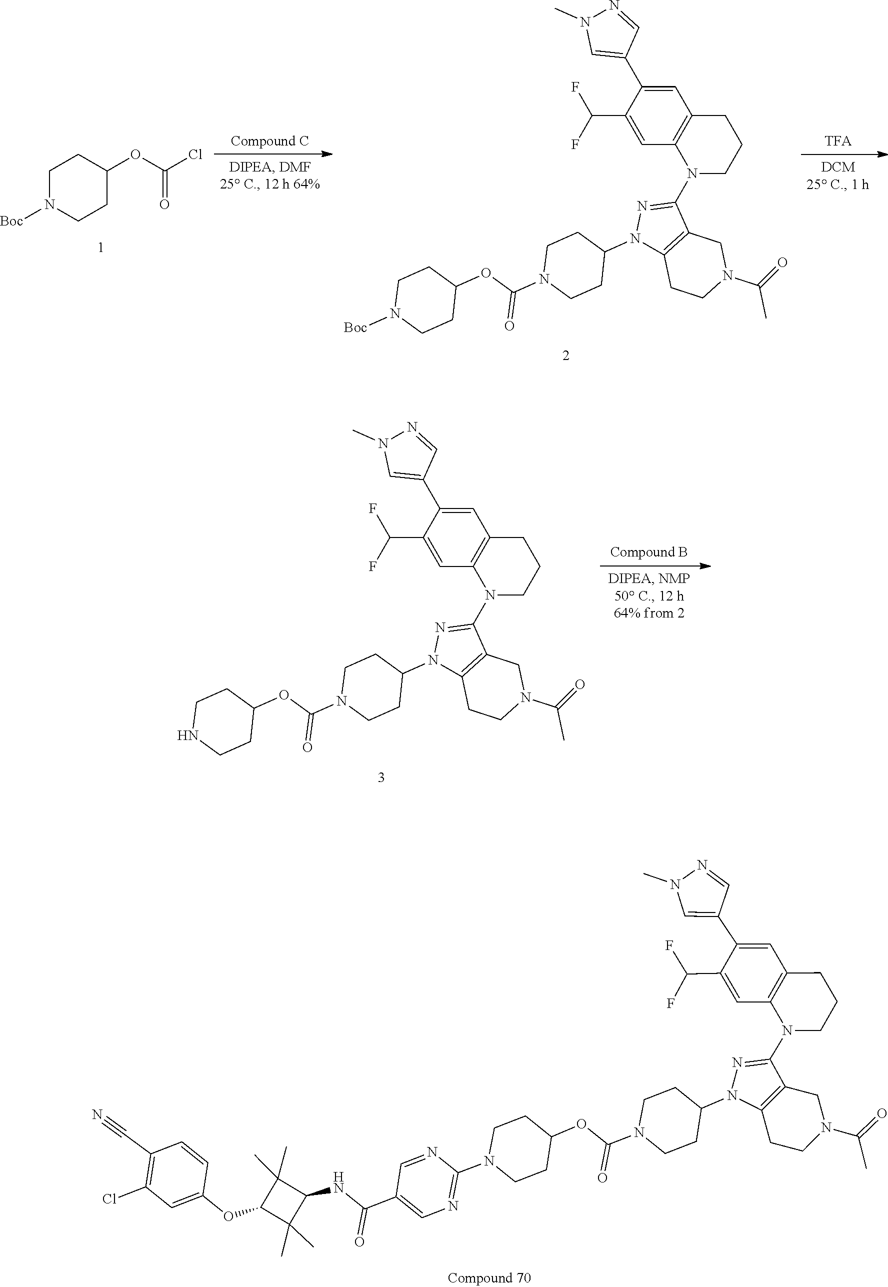 [SMILES: CC(C)(C)OC(=O)N1CCC(OC(=O)Cl)CC1 ; CC(=O)N1CCc2c(c(N3CCCc4cc(-c5cnn(C)c5)c(C(F)F)cc43)nn2C2CCN(C(=O)OC3CCN(C(=O)OC(C)(C)C)CC3)CC2)C1 ; CC(=O)N1CCc2c(c(N3CCCc4cc(-c5cnn(C)c5)c(C(F)F)cc43)nn2C2CCN(C(=O)OC3CCNCC3)CC2)C1 ; CC(=O)N1CCc2c(c(N3CCCc4cc(-c5cnn(C)c5)c(C(F)F)cc43)nn2C2CCN(C(=O)OC3CCN(c4ncc(C(=O)N[C@H]5C(C)(C)[C@H](Oc6ccc(C#N)c(Cl)c6)C5(C)C)cn4)CC3)CC2)C1]

1 was prepared according to the procedure described in WO 2018/237370 A1. To a solution of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (130 mg, 255 μmol, 1.0 eq) and DIPEA (98.9 mg, 765 μmol, 133 μL, 3.0 eq) in DMF (3 mL) was added tert-butyl 4-chlorocarbonyloxypiperidine-1-carboxylate (101 mg, 383 μmol, 1.5 eq). The mixture was stirred at 25° C. for 12 h. The mixture was diluted with EtOAc (100 mL), washed with water (10 mL×2) and brine (10 mL), dried with anhydrous sodium sulfate, filtered and concentrated under vacuum. The residue was purified by prep-TLC (silica, DCM/MeOH=20/1). Compound (1-tert-butoxycarbonyl-4-piperidyl) 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (130 mg, 162 μmol, 64% yield) was obtained as a brown solid. LC-MS: MS (ESI⁺): t_{R}=0.993 min, m/z=737.4 [M+H⁺]

To a solution of (1-tert-butoxycarbonyl-4-piperidyl) 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (130 mg, 176 mol, 1.0 eq) in DCM (6 mL) was added TFA (1.54 g, 13.5 mmol, 1 mL, 76.3 eq). The mixture was stirred at 25° C. for 1 h. The mixture was concentrated to give a residue. Compound 4-piperidyl 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (150 mg) was obtained as a brown gum and directly used in the next step without further purification. LC-MS: MS (ESI⁺): t_{R}=0.458 min, m/z=637.4 [M+H⁺]

To a solution of 4-piperidyl 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (150 mg) in NMP (3 mL) was added DIPEA (77.5 mg, 599 μmol, 104 μL, 3.0 eq) and 2-chloro-N-[3-(3-chloro-4-cyano-phenoxy)-2,2,4,4-tetramethyl-cyclobutyl]pyrimidine-5-carboxamide (100 mg, 238 μmol, 1.2 eq). The mixture was stirred at 50° C. for 12 h. The mixture was diluted with EtOAc (100 mL), washed with water (15 mL×2), brine (15 mL), dried with anhydrous sodium sulfate, filtered and concentrated under vacuum. The residue was purified by semi-preparative reverse phase HPLC (column: Phenomenex luna C18 150*25 mm*10 um; mobile phase: [water(FA)-ACN]; gradient: 63%-93% B over 10 min). Compound [1-[5-[[3-(3-chloro-4-cyano-phenoxy)-2,2,4,4-tetramethyl-cyclobutyl]carbamoyl]pyrimidin-2-yl]-4-piperidyl]4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (Compound 70, 133.17 mg, 128.59 μmol, 64% yield over two steps) was obtained as a yellow solid. ¹H NMR (400 MHz, DMSO-d₆): δ=8.76 (s, 2H) 7.89 (d, J=8.8 Hz, 1H) 7.70-7.79 (m, 2H) 7.49 (s, 1H) 7.21 (d, J=2.4 Hz, 1H) 7.10 (s, 1H) 7.00 (m, 1H) 6.63-6.93 (m, 2H) 4.89 (m, 1H) 4.28-4.37 (m, 2H) 4.02-4.19 (m, 7H) 3.86 (s, 3H) 3.68-3.81 (m, 4H) 3.55-3.62 (m, 2H) 2.94-3.09 (m, 2H) 2.73-2.89 (m, 4H) 2.07 (s, 2H) 1.93-2.01 (m, 4H) 1.88 (s, 4H) 1.59 (m, 2H) 1.19-1.24 (m, 6H) 1.11 (s, 6H) 1.05 (t, J=7.2 Hz, 1H). LC-MS: MS (ESI⁺): t_{R}=2.665 min, m/z=1019.7 [M+H⁺]

### Example 10: Synthesis of 2-[4-[2-[4-[5-acetyl-3-[2-fluoro-4-(1-methylpyrazol-4-yl)anilino]-6,7-dihydro-4H-pyrazolo[4,3-c]pyridin-1-yl]-1-piperidyl]-2-oxo-ethyl]piperazin-1-yl]-N-[3-(3-chloro-4-cyano-phenoxy)-2,2,4,4-tetramethyl-cyclobutyl]pyrimidine-5-carboxamide (Compound 66)

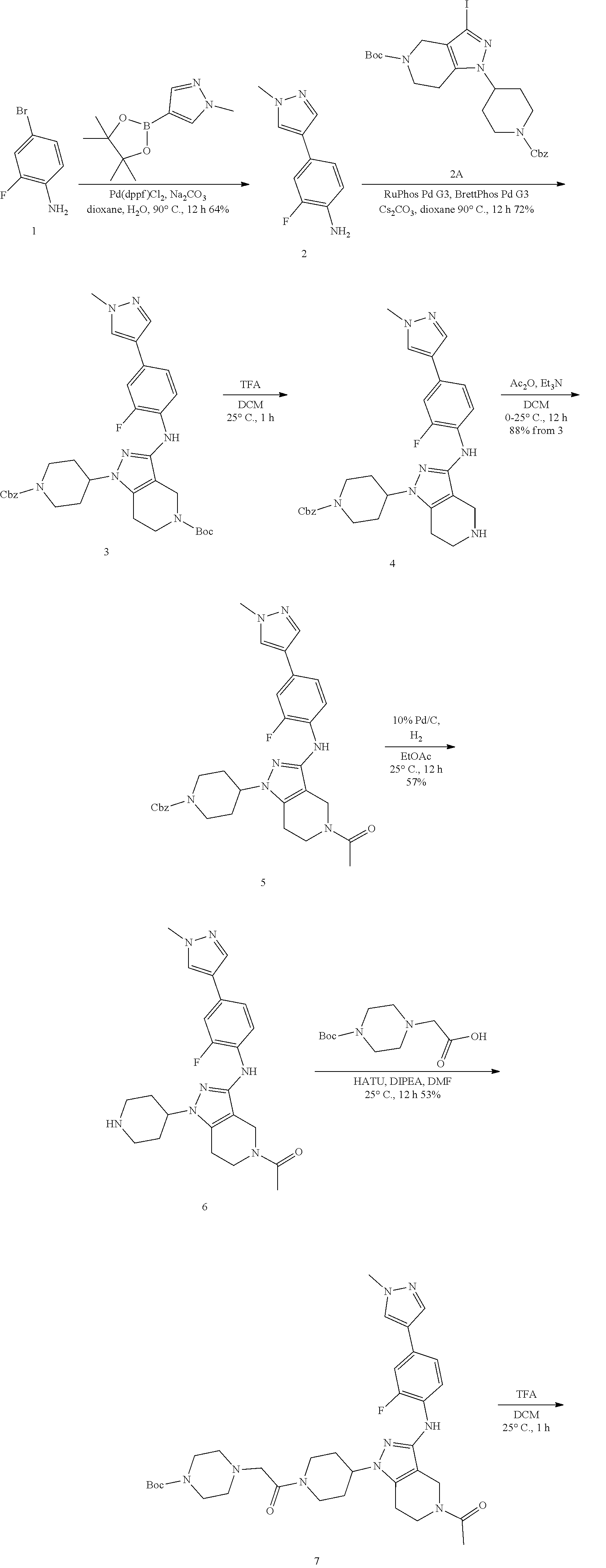

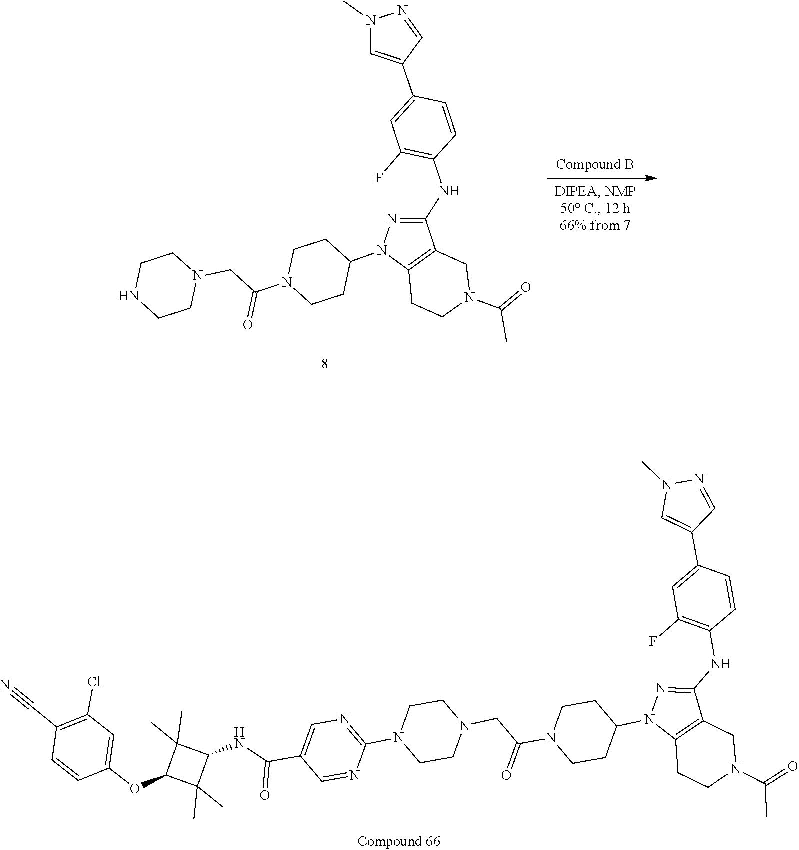 [SMILES: CC(=O)N1CCc2c(c(Nc3ccc(-c4cnn(C)c4)cc3F)nn2C2CCN(C(=O)CN3CCNCC3)CC2)C1 ; CC(=O)N1CCc2c(c(Nc3ccc(-c4cnn(C)c4)cc3F)nn2C2CCN(C(=O)CN3CCN(c4ncc(C(=O)N[C@H]5C(C)(C)[C@H](Oc6ccc(C#N)c(Cl)c6)C5(C)C)cn4)CC3)CC2)C1]

2A was prepared according to the procedure described in WO 2022/42707 A1.

To a mixture of 4-bromo-2-fluoro-aniline (5.00 g, 26.3 mmol, 1.0 eq), 1-methyl-4-(4,4,5,5-tetramethyl-1,3,2-dioxaborolan-2-yl)pyrazole (8.21 g, 39.5 mmol, 1.5 eq) and cyclopentyl(diphenyl)phosphane; dichloropalladium; iron (1.93 g, 2.63 mmol, 0.1 eq) in dioxane (50 mL) and H₂O (6 mL) was added Na₂CO₃ (8.37 g, 78.9 mmol, 3.0 eq). The mixture was stirred at 90° C. for 12 h under N₂. The mixture was poured into ice-water (150 mL). The aqueous phase was extracted with ethyl acetate (70 mL×3). The combined organic phase was washed with brine (60 mL×2), dried with anhydrous sodium sulfate, filtered and concentrated under vacuum. The residue was purified by silica gel column chromatography (petroleum ether/ethyl acetate=2/1 to 1/4). Compound 2-fluoro-4-(1-methylpyrazol-4-yl)aniline (3.5 g, 16.8 mmol, 64% yield) was obtained as an off-white solid. LC-MS: MS (ESI⁺): t_{R}=0.419 min, m/z=192.0 [M+H⁺]¹H NMR (400 MHz, CDCl₃): δ=7.65 (s, 1H) 7.49 (s, 1H) 7.01-7.15 (m, 2H) 6.73-6.82 (m, 1H) 3.92 (s, 3H)

To a solution of tert-butyl 1-(1-benzyloxycarbonyl-4-piperidyl)-3-iodo-6,7-dihydro-4H-pyrazolo[4,3-c]pyridine-5-carboxylate (0.600 g, 1.06 mmol, 1.0 eq) and 2-fluoro-4-(1-methylpyrazol-4-yl)aniline (304 mg, 1.59 mmol, 1.5 eq) in dioxane (6 mL) was added RuPhos Pd G3 (88.6 mg, 106 μmol, 0.1 eq), BrettPhos Pd G3 (96.0 mg, 106 μmol, 0.1 eq) and Cs₂CO₃ (1.04 g, 3.18 mmol, 3.0 eq). The mixture was stirred at 90° C. for 12 h under N₂. The mixture was poured into ice-water (30 mL). The aqueous phase was extracted with ethyl acetate (20 mL×3). The combined organic phase was washed with brine (20 mL), dried with anhydrous sodium sulfate, filtered and concentrated under vacuum. The residue was purified by silica gel column chromatography (petroleum ether/ethyl acetate=1/1 to 1/4). Compound tert-butyl 1-(1-benzyloxycarbonyl-4-piperidyl)-3-[2-fluoro-4-(1-methylpyrazol-4-yl)anilino]-6,7-dihydro-4H-pyrazolo[4,3-c]pyridine-5-carboxylate (0.60 g, 762 μmol, 72% yield) was obtained as a brown solid. LC-MS: MS (ESI⁺): t_{R}=0.650 min, m/z=630.5 [M+H⁺]. ¹H NMR (400 MHz, CDCl₃): δ=7.59-7.71 (m, 2H) 7.52 (s, 1H) 7.31-7.41 (m, 5H) 7.11-7.20 (m, 2H) 5.75 (d, J=2.8 Hz, 1H) 5.16 (s, 2H) 4.24-4.47 (m, 4H) 4.03 (m, 1H) 3.94 (s, 3H) 3.74 (s, 2H) 2.96 (s, 2H) 2.68 (t, J=5.6 Hz, 2H) 2.17 (m, 2H) 1.90 (d, J=11.6 Hz, 2H) 1.67 (s, 3H) 1.48 (s, 9H)

To a solution of tert-butyl 1-(1-benzyloxycarbonyl-4-piperidyl)-3-[2-fluoro-4-(1-methylpyrazol-4-yl)anilino]-6,7-dihydro-4H-pyrazolo[4,3-c]pyridine-5-carboxylate (0.450 g, 715 μmol, 1.0 eq) in DCM (5 mL) was added TFA (6.91 g, 60.6 mmol, 4.50 mL, 84.8 eq). The mixture was stirred at 25° C. for 1 h. The mixture was concentrated to give a residue. Compound benzyl 4-[3-[2-fluoro-4-(1-methylpyrazol-4-yl)anilino]-4,5,6,7-tetrahydropyrazolo[4,3-c]pyridin-1-yl]piperidine-1-carboxylate (0.46 g) was obtained as a brown solid and directly used in the next step without further purification. LC-MS: MS (ESI⁺): t_{R}=0.518 min, m/z=530.4 [M+H⁺]

To a solution of benzyl 4-[3-[2-fluoro-4-(1-methylpyrazol-4-yl)anilino]-4,5,6,7-tetrahydropyrazolo[4,3-c]pyridin-1-yl]piperidine-1-carboxylate (0.46 g) in DCM (8 mL) was added Et₃N (212 mg, 2.10 mmol, 292 μL, 3.0 eq) and Ac₂O (78.5 mg, 769 μmol, 72.2 μL, 1.1 eq) at 0° C. The mixture was stirred at 25° C. for 12 h under N₂. The mixture was poured into ice-water (40 mL). The aqueous phase was extracted with dichloromethane (30 mL×3). The combined organic phase was washed with brine (20 mL×2), dried with anhydrous sodium sulfate, filtered and concentrated under vacuum. The residue was purified by silica gel column chromatography (petroleum ether/ethyl acetate=1/1 to 0/1, then DCM/methyl alcohol=10/1). Compound benzyl 4-[5-acetyl-3-[2-fluoro-4-(1-methylpyrazol-4-yl)anilino]-6,7-dihydro-4H-pyrazolo[4,3-c]pyridin-1-yl]piperidine-1-carboxylate (0.37 g, 615 μmol, 88% yield over two steps) was obtained as a brown solid. LC-MS: MS (ESI⁺): t_{R}=0.578 min, m/z=572.4 [M+H⁺]¹H NMR (400 MHz, DMSO-d₆): δ=8.00 (s, 1H) 7.73-7.86 (m, 2H) 7.60-7.71 (m, 1H) 7.29-7.41 (m, 6H) 7.23 (t, J=10.4 Hz, 1H) 5.10 (s, 2H) 4.31-4.45 (m, 2H) 4.17-4.27 (m, 1H) 4.12 (d, J=12.4 Hz, 2H) 3.83 (s, 3H) 3.64-3.76 (m, 2H) 2.92-3.10 (m, 2H) 2.74-2.82 (m, 1H) 2.66 (m, 1H) 2.07 (d, J=13.2 Hz, 3H) 1.81-1.92 (m, 4H).

Pd/C (0.2 g, 10% Pd on carbon, w/w) was added into a 30 mL single-necked round bottom flask under N₂, and then EtOAc (10 mL) was added at 25° C. under N₂. After addition, benzyl 4-[5-acetyl-3-[2-fluoro-4-(1-methylpyrazol-4-yl)anilino]-6,7-dihydro-4H-pyrazolo[4,3-c]pyridin-1-yl]piperidine-1-carboxylate (0.370 g, 647 μmol, 1.0 eq) was added under N₂. The suspension was degassed under vacuum and purged with H₂ several times. The mixture was stirred under H₂ (15 psi) at 25° C. for 1.5 h. The reaction mixture was filtered and washed with ethanol (20 mL×2). The collected filtrate was concentrated to give a residue. The residue was purified by semi-preparative reverse phase HPLC (column: Phenomenex luna C18 150*25 mm*10 um; mobile phase: [water(FA)-ACN]; gradient: 8%-38% B over 10 min). Compound 1-[3-[2-fluoro-4-(1-methylpyrazol-4-yl)anilino]-1-(4-piperidyl)-6,7-dihydro-4H-pyrazolo[4,3-c]pyridin-5-yl]ethanone (0.160 g, 366 μmol, 57% yield) was obtained as a white solid. LC-MS: MS (ESI⁺): t_{R}=0.732 min, m/z=438.3 [M+H⁺]

To a solution of 2-(4-tert-butoxycarbonylpiperazin-1-yl)acetic acid (67.0 mg, 274 μmol, 1.2 eq) in DMF (1.5 mL) was added HATU (130 mg, 343 μmol, 1.5 eq) and stirred at 25° C. for 0.5 h. 1-[3-[2-fluoro-4-(1-methylpyrazol-4-yl)anilino]-1-(4-piperidyl)-6,7-dihydro-4H-pyrazolo[4,3-c]pyridin-5-yl]ethanone (100 mg, 229 μmol, 1.0 eq) and DIPEA (88.6 mg, 686 μmol, 119 μL, 3.0 eq) in DMF (0.5 mL) was added to the above reaction mixture and stirred at 25° C. for 11.5 h. The mixture was diluted with EtOAc (100 mL), washed with water (15 mL×2), brine (15 mL), dried with anhydrous sodium sulfate, filtered and concentrated under vacuum. The residue was purified by semi-preparative reverse phase HPLC (column: Phenomenex luna C18 150*25 mm*10 um; mobile phase: [water(FA)-ACN]; gradient: 18%-48% B over 10 min). Compound tert-butyl 4-[2-[4-[5-acetyl-3-[2-fluoro-4-(1-methylpyrazol-4-yl)anilino]-6,7-dihydro-4H-pyrazolo[4,3-c]pyridin-1-yl]-1-piperidyl]-2-oxo-ethyl]piperazine-1-carboxylate (80.0 mg, 121 μmol, 53% yield) was obtained as a white solid. LC-MS: MS (ESI⁺): t_{R}=0.496 min, m/z=664.5 [M+H⁺]

To a solution of tert-butyl 4-[2-[4-[5-acetyl-3-[2-fluoro-4-(1-methylpyrazol-4-yl)anilino]-6,7-dihydro-4H-pyrazolo[4,3-c]pyridin-1-yl]-1-piperidyl]-2-oxo-ethyl]piperazine-1-carboxylate (80.0 mg, 121 μmol, 1.0 eq) in DCM (3 mL) was added TFA (1.54 g, 13.5 mmol, 1 mL, 111.7 eq) and stirred at 25° C. for 1 h. The mixture was concentrated to give a residue. Compound 1-[4-[5-acetyl-3-[2-fluoro-4-(1-methylpyrazol-4-yl)anilino]-6,7-dihydro-4H-pyrazolo[4,3-c]pyridin-1-yl]-1-piperidyl]-2-piperazin-1-yl-ethanone (80 mg) was obtained as a brown gum and directly used in the next step without further purification. LC-MS: MS (ESI⁺): t_{R}=0.741 min, m/z=564.3 [M+H⁺]

To a solution of 1-[4-[5-acetyl-3-[2-fluoro-4-(1-methylpyrazol-4-yl)anilino]-6,7-dihydro-4H-pyrazolo[4,3-c]pyridin-1-yl]-1-piperidyl]-2-piperazin-1-yl-ethanone (80 mg) in NMP (3 mL) was added DIPEA (76.3 mg, 590 μmol, 103 μL, 5.0 eq) and 2-chloro-N-[3-(3-chloro-4-cyano-phenoxy)-2,2,4,4-tetramethyl-cyclobutyl]pyrimidine-5-carboxamide (54.5 mg, 130 μmol, 1.1 eq). The reaction mixture was stirred at 50° C. for 12 h. The mixture was diluted with EtOAc (100 mL), washed with water (15 mL×2), brine (15 mL), dried with anhydrous sodium sulfate, filtered and concentrated under vacuum. The residue was purified by semi-preparative reverse phase HPLC (column: Phenomenex luna C18 150*25 mm*10 um; mobile phase: [water (FA)-ACN]: gradient: 30%-60% B over 10 min). Compound 2-[4-[2-[4-[5-acetyl-3-[2-fluoro-4-(1-methylpyrazol-4-yl) anilino]-6,7-dihydro-4H-pyrazolo[4,3-c]pyridin-1-yl]-1-piperidyl]-2-oxo-ethyl]piperazin-1-yl]-N-[3-(3-chloro-4-cyano-phenoxy)-2,2,4,4-tetramethyl-cyclobutyl]pyrimidine-5-carboxamide (Compound 66, 74.3 mg, 78.2 μmol, 66% yield over two steps) was obtained as a white solid. ¹H NMR (400 MHZ, DMSO-d₆): δ=8.72 (s, 2H) 8.29 (s, 1H) 7.94 (s, 1H) 7.87-7.92 (m, 1H) 7.68-7.78 (m, 3H) 7.33 (m, 1H) 7.16-7.28 (m, 2H) 7.00 (m, 1H) 4.48 (d, J=12.4 Hz, 1H) 4.33-4.44 (m, 2H) 4.24-4.33 (m, 2H) 4.13-4.23 (m, 1H) 4.03 (d, J=8.8 Hz, 1H) 3.82 (s, 2H) 3.87 (s, 2H) 3.65-3.78 (m, 2H) 3.42 (m, 7H) 3.08-3.21 (m, 2H) 2.67-2.82 (m, 3H) 2.07 (br d, J=13.6 Hz, 3H) 1.72-1.98 (m, 3H) 1.21 (s, 6H) 1.11 (s, 6H). LC-MS: MS (ESI⁺): t_{R}=2.195 min, m/z=946.7 [M+H⁺]

The compounds below were prepared in a similar manner as described in Example 10.

| | | | LC-MS |
|---|---|---|---|
| Compound | Name | ¹H NMR (CDCl₃) | [M + H⁺] |
| 1 | 2-[4-[2-[2-[5-acetyl-3-[2-fluoro- | δ = 8.70 (d, J = 1.2 Hz, 2H), 8.33 (s, 1H), | 893.5 |
| | 4-(1-methylpyrazol-4- | 7.72-7.39 (m, 4H), 7.20-7.10 (m, 2H), | |
| | yl)anilino]-6,7-dihydro-4H- | 6.97 (d, J = 2.4 Hz, 1H), 6.81 (dd, J = | |
| | pyrazolo[4,3-c]pyridin-1- | 2.4, 8.4 Hz, 1H), 6.22-5.97 (m, 1H), | |
| | yl]ethoxy]ethyl]piperazin-1-yl]- | 5.81 (dd, J = 3.2, 18.0 Hz, 1H), 4.46 (s, | |
| | N-[3-(3-chloro-4-cyano- | 1H), 4.31 (s, 1H), 4.18-4.02 (m, 4H), | |
| | phenoxy)-2,2,4,4-tetramethyl- | 3.93 (d, J = 1.6 Hz, 3H), 3.88 (quin, J = | |
| | cyclobutyl]pyrimidine-5- | 4.8 Hz, 5H), 3.81 (q, J = 5.6 Hz, 2H), | |
| | carboxamide | 3.71 (br t, J = 6.0 Hz, 1H), 3.59 (br t, J = | |
| | | 4.0 Hz, 2H), 2.84-2.71 (m, 2H), 2.59 (t, | |
| | | J = 5.2 Hz, 2H), 2.47 (td, J = 5.2, 10.4 | |
| | | Hz, 4H), 2.16 (d, J = 18.8 Hz, 3H), 1.32-1.17 | |
| | | (m, 12H). | |
| 2 | 2-[4-[2-[5-acetyl-3-[2-fluoro-4- | δ = 8.69 (d, J = 2.8 Hz, 2H), 7.68 (s, 1H), | 432.9 |
| | (1-methylpyrazol-4-yl)anilino]- | 7.64-7.36 (m, 3H), 7.22-7.11 (m, 2H), | [M/2 + H+] |
| | 6,7-dihydro-4H-pyrazolo[4,3- | 6.97 (d, J = 2.4 Hz, 1H), 6.81 (dd, J = | |
| | c]pyridin-1-yl]ethoxy]-1- | 2.4, 8.8 Hz, 1H), 6.24-5.91 (m, 1H), | |
| | piperidyl]-N-[3-(3-chloro-4- | 5.77 (dd, J = 2.8, 17.2 Hz, 1H), 4.47-4.27 | |
| | cyano-phenoxy)-2,2,4,4- | (m, 2H), 4.17-4.10 (m, 3H), 4.10-4.01 | |
| | tetramethyl- | (m, 3H), 3.94 (d, J = 2.0 Hz, 3H), | |
| | cyclobutyl]pyrimidine-5- | 3.90-3.81 (m, 3H), 3.69 (t, J = 5.6 Hz, | |
| | carboxamide | 1H), 3.66-3.53 (m, 3H), 2.78 (td, J = | |
| | | 5.6, 18.0 Hz, 2H), 2.12 (d, J = 6.4 Hz, | |
| | | 3H), 1.82 (dt, J = 3.6, 6.4 Hz, 2H), 1.59-1.46 | |
| | | (m, 2H), 1.23 (d, J = 14.4 Hz, 12H). | |
| 3 | 2-[4-[2-[3-[5-acetyl-3-[2-fluoro- | δ = 8.72-8.63 (m, 2H), 7.83-7.46 (m, | 903.5 |
| | 4-(1-methylpyrazol-4- | 4H), 7.24-7.14 (m, 2H), 6.96 (d, J = 2.4 | |
| | yl)anilino]-6,7-dihydro-4H- | Hz, 1H), 6.81 (dd, J = 2.4, 8.8 Hz, 1H), | |
| | pyrazolo[4,3-c]pyridin-1- | 6.16-5.74 (m, 2H), 5.06-4.74 (m, 3H), | |
| | yl]azetidin-1-yl]ethyl]-1- | 4.46 (s, 1H), 4.36-4.26 (m, 1H), 4.21 | |
| | piperidyl]-N-[3-(3-chloro-4- | (br t, J = 8.0 Hz, 1H), 4.16-4.02 (m, | |
| | cyano-phenoxy)-2,2,4,4- | 3H), 3.98-3.81 (m, 5H), 3.73 (br t, J = | |
| | tetramethyl- | 5.6 Hz, 2H), 3.02-2.85 (m, 4H), 2.75-2.59 | |
| | cyclobutyl]pyrimidine-5- | (m, 5H), 2.26-2.08 (m, 3H), 1.83 | |
| | carboxamide | (br d, J = 12.8 Hz, 2H), 1.55-1.42 (m, | |
| | | 2H), 1.25 (s, 6H), 1.22 (s, 6H) | |
| 4 | 2-[4-[[3-[5-acetyl-3-[2-fluoro-4- | δ = 8.70 (s, 2H), 7.4-7.8 (m, 4H), 7.1-7.2 | 889.5 |
| | (1-methylpyrazol-4-yl)anilino]- | (m, 2H), 6.97 (d, 1H, J = 2.0 Hz), 6.81 | |
| | 6,7-dihydro-4H-pyrazolo[4,3- | (dd, 1H, J = 2.1, 8.8 Hz), 5.8-6.1 (m, 2H), | |
| | c]pyridin-1-yl]azetidin-1- | 5.0-5.2 (m, 1H), 4.8-5.0 (m, 2H), 4.6-4.7 | |
| | yl]methyl]-1-piperidyl]-N-[3-(3- | (m, 1H), 4.4-4.6 (m, 2H), 4.28 (s, 1H), | |
| | chloro-4-cyano-phenoxy)- | 4.1-4.2 (m, 2H), 4.0-4.1 (m, 2H), 3.8-4.0 | |
| | 2,2,4,4-tetramethyl- | (m, 4H), 3.74 (t, 1H, J = 5.6 Hz), 3.13 (d, | |
| | cyclobutyl]pyrimidine-5- | 1H, J = 5.5 Hz), 2.97 (t, 3H, J = 12.8 Hz), | |
| | carboxamide | 2.6-2.8 (m, 2H), 2.1-2.3 (m, 4H), 1.8-2.0 | |
| | | (m, 4H), 1.24 (d, 12H, J = 15.8 Hz) | |
| 5 | 2-[4-[3-[5-acetyl-3-[2-fluoro-4- | δ = 8.71 (d, J = 1.6 Hz, 2H), 8.30 (s, 1H), | 875.5 |
| | (1-methylpyrazol-4-yl)anilino]- | 7.78-7.43 (m, 4H), 7.24-7.15 (m, 2H), | |
| | 6,7-dihydro-4H-pyrazolo[4,3- | 6.97 (d, J = 2.4 Hz, 1H), 6.81 (dd, J = | |
| | c]pyridin-1-yl]azetidin-1-yl]-1- | 2.4, 8.8 Hz, 1H), 5.97 (dd, J = 5.2, 8.0 | |
| | piperidyl]-N-[3-(3-chloro-4- | Hz, 1H), 5.92-5.81 (m, 1H), 5.10-4.74 | |
| | cyano-phenoxy)-2,2,4,4- | (m, 3H), 4.46 (s, 1H), 4.28 (s, 1H), 4.22-4.07 | |
| | tetramethyl- | (m, 3H), 4.05 (s, 1H), 3.99 (br t, J = | |
| | cyclobutyl]pyrimidine-5- | 8.4 Hz, 1H), 3.95 (s, 3H), 3.93-3.83 (m, | |
| | carboxamide | 2H), 3.74 (br t, J = 5.2 Hz, 1H), 3.21-3.07 | |
| | | (m, 2H), 3.05-2.85 (m, 1H), 2.79-2.67 | |
| | | (m, 2H), 2.20-2.13 (m, 3H), 2.02-1.91 | |
| | | (m, 2H), 1.58-1.41 (m, 2H), 1.24 | |
| | | (d, J = 14.8 Hz, 12H). | |
| 6 | 2-[4-[2-[3-[5-acetyl-3-[2-fluoro- | δ = 8.69 (s, 2H), 7.68 (s, 1H), 7.63-7.35 | 917.5 |
| | 4-(1-methylpyrazol-4- | (m, 2H), 7.38 (br t, J = 8.8 Hz, 1H), 7.23-7.08 | |
| | yl)anilino]-6,7-dihydro-4H- | (m, 2H), 6.96 (d, J = 2.4 Hz, 1H), | |
| | pyrazolo[4,3-c]pyridin-1- | 6.80 (dd, J = 2.4, 8.8 Hz, 1H), 5.99 (br | |
| | yl]pyrrolidin-1-yl]ethyl]-1- | dd, J = 5.2, 7.6 Hz, 1H), 5.89-5.77 (m, | |
| | piperidyl]-N-[3-(3-chloro-4- | 1H), 4.83 (br d, J = 13.2 Hz, 3H), 4.46 | |
| | cyano-phenoxy)-2,2,4,4- | (s, 1H), 4.28 (s, 1H), 4.13 (br d, J = 8.4 | |
| | tetramethyl- | Hz, 1H), 4.04 (s, 1H), 3.97-3.88 (m, | |
| | cyclobutyl]pyrimidine-5- | 4H), 3.75-3.47 (m, 2H), 3.49 (br t, J = | |
| | carboxamide | 8.8 Hz, 1H), 3.38-3.17 (m, 2H), 3.06-3.02 | |
| | | (m, 1H), 2.96-2.86 (m, 4H), 2.83-2.70 | |
| | | (m, 3H), 2.51-2.38 (m, 1H), 2.36-2.25 | |
| | | (m, 1H), 2.22-2.09 (m, 3H), 1.80 | |
| | | (br d, J = 13.2 Hz, 2H), 1.66-1.57 (m, | |
| | | 2H), 1.25 (s, 6H), 1.21 (s, 7H) | |
| 7 | 2-[4-[[3-[5-acetyl-3-[2-fluoro-4- | δ = 8.70 (s, 2H), 7.70 (d, J = 2.0 Hz, 1H), | 903.5 |
| | (1-methylpyrazol-4-yl)anilino]- | 7.69-7.40 (m, 3H), 7.22-7.09 (m, 2H), | |
| | 6,7-dihydro-4H-pyrazolo[4,3- | 6.97 (d, J = 2.4 Hz, 1H), 6.81 (dd, J = | |
| | c]pyridin-1-yl]pyrrolidin-1- | 2.4, 8.8 Hz, 1H), 6.00 (br d, J = 8.0 Hz, | |
| | yl]methyl]-1-piperidyl]-N-[3-(3- | 1H), 5.83 (dd, J = 2.8, 13.6 Hz, 1H), 4.87 | |
| | chloro-4-cyano-phenoxy)- | (br d, J = 12.0 Hz, 2H), 4.83-4.70 (m, | |
| | 2,2,4,4-tetramethyl- | 1H), 4.53-4.39 (m, 1H), 4.28 (s, 1H), | |
| | cyclobutyl]pyrimidine-5- | 4.13 (d, J = 8.0 Hz, 1H), 4.05 (s, 1H), | |
| | carboxamide | 3.94 (s, 3H), 3.93-3.83 (m, 1H), 3.75 | |
| | | (br t, J = 5.6 Hz, 1H), 3.48-3.26 (m, | |
| | | 1H), 3.20-3.01 (m, 1H), 3.01-2.88 (m, | |
| | | 4H), 2.81-2.71 (m, 2H), 2.63-2.51 (m, | |
| | | 4H), 2.44-2.25 (m, 2H), 2.22-2.10 (m, | |
| | | 3H), 2.01-1.81 (m, 3H), 1.24 (d, J = | |
| | | 15.6 Hz, 12H). | |
| 8 | 2-[4-[3-[5-acetyl-3-[2-fluoro-4- | δ = 8.70 (s, 2H), 8.35 (s, 1H), 7.69 (s, | 889.5 |
| | (1-methylpyrazol-4-yl)anilino]- | 1H), 7.68-7.38 (m, 3H), 7.21-7.11 (m, | |
| | 6,7-dihydro-4H-pyrazolo[4,3- | 2H), 6.97 (d, J = 2.4 Hz, 1H), 6.81 (dd, | |
| | c]pyridin-1-yl]pyrrolidin-1-yl]- | J = 2.4, 8.8 Hz, 1H), 5.99 (dd, J = 5.2, 8.0 | |
| | 1-piperidyl]-N-[3-(3-chloro-4- | Hz, 1H), 5.82 (dd, J = 2.8, 16.8 Hz, 1H), | |
| | cyano-phenoxy)-2,2,4,4- | 4.96-4.74 (m, 3H), 4.50-4.24 (m, 2H), | |
| | tetramethyl- | 4.13 (d, J = 8.0 Hz, 1H), 4.05 (s, 1H), | |
| | cyclobutyl]pyrimidine-5- | 3.94 (s, 3H), 3.93-3.84 (m, 1H), 3.75 | |
| | carboxamide | (br t, J = 6.0 Hz, 1H), 3.73-3.51 (m, | |
| | | 1H), 3.39-3.18 (m, 1H), 3.16-2.99 (m, | |
| | | 4H), 2.96-2.70 (m, 3H), 2.44-2.30 (m, | |
| | | 2H), 2.20 (br s, 3H), 2.09-2.00 (m, 2H), | |
| | | 1.76-1.60 (m, 2H), 1.24 (d, J = 15.6 Hz, | |
| | | 12H). | |
| 164 | 2-[4-[2-[3-[5-acetyl-3-[2-fluoro- | δ = 8.71 (s, 2H), 7.78-7.46 (m, 4H), | 452.9 |
| | 4-(1-methylpyrazol-4- | 7.23-7.15 (m, 2H), 6.96 (d, J = 2.4 Hz, | [M/2] |
| | yl)anilino]-6,7-dihydro-4H- | 1H), 6.80 (dd, J = 2.4, 8.8 Hz, 1H), 5.97 | |
| | pyrazolo[4,3-c]pyridin-1- | (dd, J = 3.2, 8.4 Hz, 1H), 5.87 (dd, J = | |
| | yl]azetidin-1-yl]ethyl]piperazin- | 2.0, 17.6 Hz, 1H), 4.96-4.79 (m, 1H), | |
| | 1-yl]-N-[3-(3-chloro-4-cyano- | 4.45 (s, 1H), 4.27 (s, 1H), 4.13 (br d, J = | |
| | phenoxy)-2,2,4,4-tetramethyl- | 8.0 Hz, 2H), 4.05-3.82 (m, 10H), 3.74 | |
| | cyclobutyl]pyrimidine-5- | (br t, J = 5.2 Hz, 2H), 2.99 (br t, J = 5.2 | |
| | carboxamide | Hz, 1H), 2.91 (br t, J = 6.0 Hz, 1H), 2.73 | |
| | | (br t, J = 5.2 Hz, 1H), 2.67 (br t, J = 5.6 | |
| | | Hz, 1H), 2.63-2.50 (m, 6H), 2.19-2.12 | |
| | | (m, 3H), 1.24 (d, J = 14.4 Hz, 12H) | |
| 166 | 2-[4-[2-[3-[5-acetyl-3-[2-fluoro- | δ = 8.67 (s, 2H), 7.96-7.64 (m, 2H), | 459.4 |
| | 4-(1-methylpyrazol-4- | 7.61-7.53 (m, 2H), 7.24-7.14 (m, 2H), | [M/2] |
| | yl)anilino]-6,7-dihydro-4H- | 6.96 (d, J = 2.4 Hz, 1H), 6.80 (dd, J = | |
| | pyrazolo[4,3-c]pyridin-1- | 2.4, 8.8 Hz, 1H), 6.06-5.96 (m, 1H), | |
| | yl]azetidin-1-yl]-2-oxo-ethyl]-1- | 5.95-5.83 (m, 1H), 4.96-4.80 (m, 3H), | |
| | piperidyl]-N-[3-(3-chloro-4- | 4.68 (ddd, J = 5.2, 8.4, 14.0 Hz, 1H), | |
| | cyano-phenoxy)-2,2,4,4- | 4.58-4.36 (m, 4H), 4.31 (s, 1H), 4.13 | |
| | tetramethyl- | (d, J = 8.4 Hz, 1H), 4.05 (s, 1H), 3.93 (d, | |
| | cyclobutyl]pyrimidine-5- | J = 2.4 Hz, 4H), 3.82-3.67 (m, 1H), | |
| | carboxamide | 2.97 (br t, J = 12.4 Hz, 2H), 2.78-2.62 | |
| | | (m, 2H), 2.30-2.06 (m, 5H), 2.02-1.75 | |
| | | (m, 3H), 1.23 (d, J = 16.0 Hz, 14H) | |
| 167 | 2-[4-[2-[4-[5-acetyl-3-[2-fluoro- | δ = 8.78 (s, 2H), 8.01 (s, 1H), 7.92-7.69 | 932.5 |
| | 4-(1-methylpyrazol-4- | (m, 5H), 7.36 (ddd, J = 2.0, 8.7, 13.2 Hz, | |
| | yl)anilino]-6,7-dihydro-4H- | 1H), 7.26-7.16 (m, 2H), 7.01 (dd, J = | |
| | pyrazolo[4,3-c]pyridin-1-yl]-1- | 2.4, 8.8 Hz, 1H), 4.42 (s, 1H), 4.35 (s, | |
| | piperidyl]ethyl]piperazin-1-yl]- | 1H), 4.29 (s, 1H), 4.04 (d, J = 9.2 Hz, | |
| | N-[3-(3-chloro-4-cyano- | 1H), 3.86 (br s, 4H), 3.83 (s, 3H), 3.77-3.63 | |
| | phenoxy)-2,2,4,4-tetramethyl- | (m, 3H), 3.43-3.30 (m, 5H), 2.85-2.72 | |
| | cyclobutyl]pyrimidine-5- | (m, 2H), 2.66 (br s, 3H), 2.55 (br s, | |
| | carboxamide | 4H), 2.28-2.12 (m, 2H), 2.07 (d, J = | |
| | | 13.2 Hz, 3H), 2.02-1.83 (m, 2H), 1.21 | |
| | | (s, 6H), 1.11 (s, 6H) | |
| 168 | 2-[4-[2-[4-[5-acetyl-3-[2-fluoro- | δ = 8.70 (s, 2 H) 7.69 (d, J = 2.40 Hz, 1 H) | 931.6 |
| | 4-(1-methylpyrazol-4- | 7.58 (d, J = 8.80 Hz, 1 H) 7.55 (d, J = 3.60 | |
| | yl)anilino]-6,7-dihydro-4H- | Hz, 1 H) 7.11-7.24 (m, 2 H) 6.97 (d, | |
| | pyrazolo[4,3-c]pyridin-1-yl]-1- | J = 2.40 Hz, 1 H) 6.81 (dd, J = 8.80, 2.40 | |
| | piperidyl]ethyl]-1-piperidyl]-N- | Hz, 1 H) 5.97 (br dd, J = 7.20, 4.80 Hz, | |
| | [3-(3-chloro-4-cyano-phenoxy)- | 1 H) 5.70-5.83 (m, 1 H) 4.87 (br s, 1 H) | |
| | 2,2,4,4-tetramethyl- | 4.84 (br s, 1 H) 4.48 (s, 1 H) 4.30 (s, | |
| | cyclobutyl]pyrimidine-5- | 2 H) 4.13 (d, J = 8.00 Hz, 1 H) 4.05 (s, 1 H) | |
| | carboxamide | 3.95 (s, 3 H) 3.88-3.94 (m, 1 H) 3.73-3.80 | |
| | | (m, 1 H) 3.57-3.73 (m, 2 H) 3.19-3.43 | |
| | | (m, 1 H) 3.03-3.15 (m, 2 H) 2.92 | |
| | | (br t, J = 12.40 Hz, 2 H) 2.73-2.80 (m, | |
| | | 1 H) 2.69 (br t, J = 5.20 Hz, 1 H) 2.27-2.56 | |
| | | (m, 3 H) 2.21 (s, 2 H) 2.15 (s, 2 H) 1.82 | |
| | | (br s, 3 H) 1.78 (br d, J = 6.80 Hz, 4 H) | |
| | | 1.26 (s, 6 H) | |
| 177 | 2-[4-[2-[4-[5-acetyl-3-[2-fluoro- | δ = 8.74-8.63 (m, 2H), 7.82-7.49 (m, | 961.7 |
| | 4-(1-methylpyrazol-4- | 4H), 7.22-7.09 (m, 2H), 6.97 (d, J = 2.4 | |
| | yl)anilino]-6, 7-dihydro-4H- | Hz, 1H), 6.81 (dd, J = 2.4, 8.8 Hz, 1H), | |
| | pyrazolo[4,3-c]pyridin-1-yl]-1- | 6.11-5.98 (m, 1H), 5.79 (dd, J = 3.2, | |
| | piperidyl]-2-oxo-ethoxy]-1- | 19.2 Hz, 1H), 4.71 (br d, J = 13.6 Hz, | |
| | piperidyl]-N-[3-(3-chloro-4- | 1H), 4.59-4.40 (m, 1H), 4.37-4.26 (m, | |
| | cyano-phenoxy)-2,2,4,4- | 5H), 4.22-4.04 (m, 4H), 3.96-3.87 (m, | |
| | tetramethyl- | 4H), 3.76 (br d, J = 3.2 Hz, 2H), 3.67-3.55 | |
| | cyclobutyl]pyrimidine-5- | (m, 2H), 3.21 (br t, J = 12.4 Hz, | |
| | carboxamide | 1H), 2.90-2.68 (m, 3H), 2.31-2.09 (m, | |
| | | 5H), 1.99 (br s, 4H), 1.75-1.69 (m, 2H), | |
| | | 1.26 (s, 6H), 1.22 (s, 6H) | |
| 178 | 2-[4-[2-[4-[5-acetyl-3-[2-fluoro- | δ = 8.69 (s, 2 H) 7.70 (s, 1 H) 7.57 (d, | 945.6 |
| | 4-(1-methylpyrazol-4- | J = 8.80 Hz, 1 H) 7.53 (d, J = 4.00 Hz, 1 H) | |
| | yl)anilino]-6,7-dihydro-4H- | 7.46 (t, J = 8.80 Hz, 1 H) 7.12-7.21 (m, | |
| | pyrazolo[4,3-c]pyridin-1-yl]-1- | 2 H) 6.97 (d, J = 2.40 Hz, 1 H) 6.81 (dd, | |
| | piperidyl]-2-oxo-ethyl]-1- | J = 8.80, 2.40 Hz, 1 H) 5.96 (br d, J = 8.40 | |
| | piperidyl]-N-[3-(3-chloro-4- | Hz, 1 H) 5.79 (dd, J = 16.80, 3.20 Hz, | |
| | cyano-phenoxy)-2,2,4,4- | 1 H) 4.88 (br d, J = 12.80 Hz, 2 H) 4.80 (br | |
| | tetramethyl- | d, J = 14.00 Hz, 1 H) 4.40-4.55 (m, 1 H) | |
| | cyclobutyl]pyrimidine-5- | 4.30 (s, 1 H) 4.13 (d, J = 8.00 Hz, 1 H) | |
| | carboxamide | 4.05 (s, 1 H) 3.93 (d, J = 2.80 Hz, 4 H) | |
| | | 3.76 (br d, J = 5.60 Hz, 1 H) 3.20 (br t, | |
| | | J = 12.00 Hz, 1 H) 3.00 (br t, J = 12.40 Hz, | |
| | | 2 H) 2.67-2.85 (m, 3 H) 2.30-2.38 (m, | |
| | | 2 H) 2.21 (s, 2 H) 2.15 (s, 2 H) 1.90-1.99 | |
| | | (m, 4 H) 1.76 (br d, J = 1.60 Hz, 6 H) | |
| | | 1.25 (s, 6 H) 1.22 (s, 6 H). | |
| 186 | 2-[4-[[4-[5-acetyl-3-[2-fluoro-4- | δ = 8.70 (s, 2H), 7.4-7.7 (m, 4H), 7.1-7.2 | 917.5 |
| | (1-methylpyrazol-4-yl)anilino]- | (m, 2H), 6.97 (d, 1H, J = 2.4 Hz), 6.81 | |
| | 6,7-dihydro-4H-pyrazolo[4,3- | (dd, 1H, J = 2.4, 8.8 Hz), 5.9-6.0 (m, 1H), | |
| | c]pyridin-1-yl]-1- | 5.7-5.8 (m, 1H), 4.87 (d, 2H, J = 12.4 Hz), | |
| | piperidyl]methyl]-1-piperidyl]- | 4.47 (s, 1H), 4.30 (s, 1H), 4.13 (d, 1H, | |
| | N-[3-(3-chloro-4-cyano- | J = 8.1 Hz), 4.05 (s, 1H), 3.9-4.0 (m, 5H), | |
| | phenoxy)-2,2,4,4-tetramethyl- | 3.75 (t, 1H, J = 5.6 Hz), 3.0-3.3 (m, 2H), | |
| | cyclobutyl]pyrimidine-5- | 2.96 (t, 2H, J = 12.4 Hz), 2.7-2.8 (m, 2H), | |
| | carboxamide | 2.3-2.4 (m, 4H), 2.2-2.2 (m, 2H), 2.14 (s, | |
| | | 2H), 1.94 (d, 5H, J = 9.6 Hz), 1.7-1.8 (m, | |
| | | 3H), 1.24 (d, 12H, J = 14.8 Hz) | |

### Example 11: Synthesis of 2-[4-[2-[3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidin-1-yl]-2-oxo-ethyl]piperazin-1-yl]-N-[3-(3-chloro-4-cyano-phenoxy)-2,2,4,4-tetramethyl-cyclobutyl]pyrimidine-5-carboxamide (Compound 65)

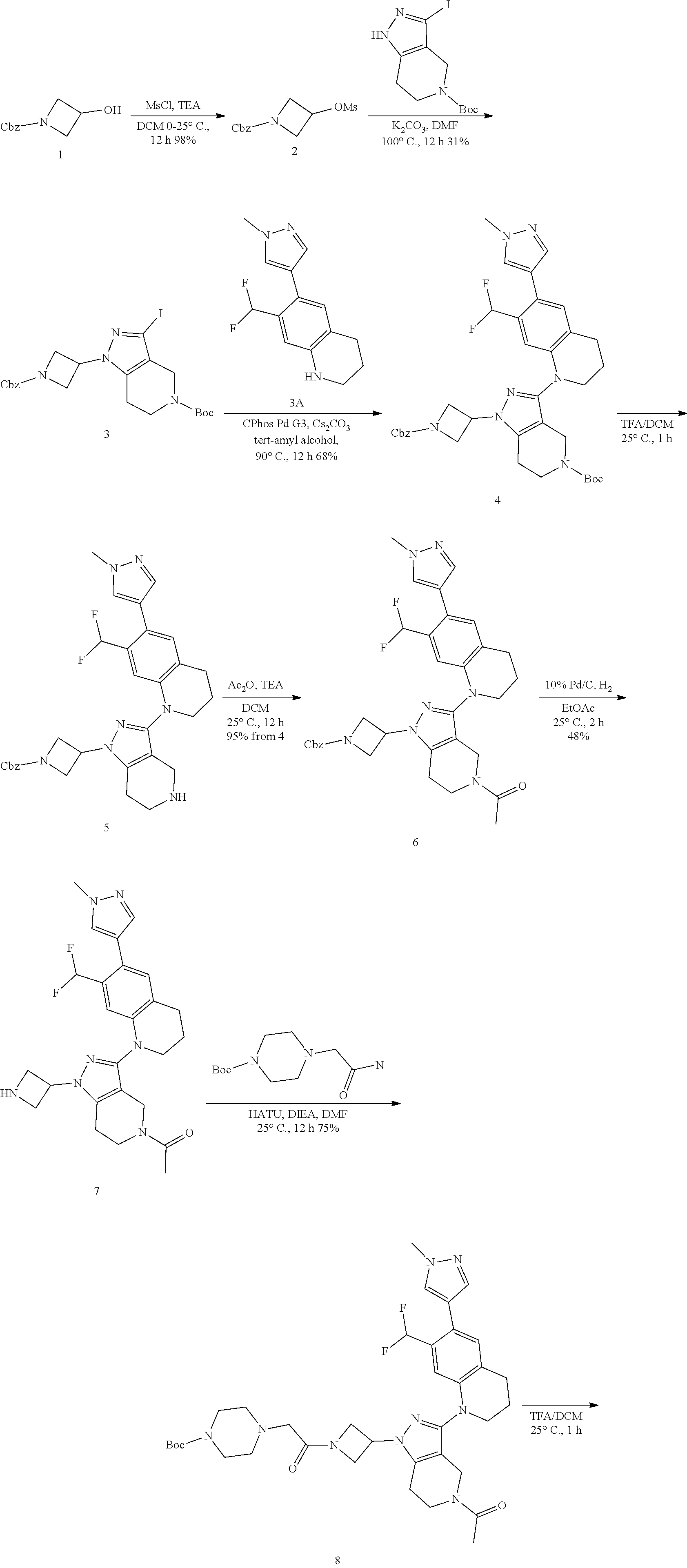

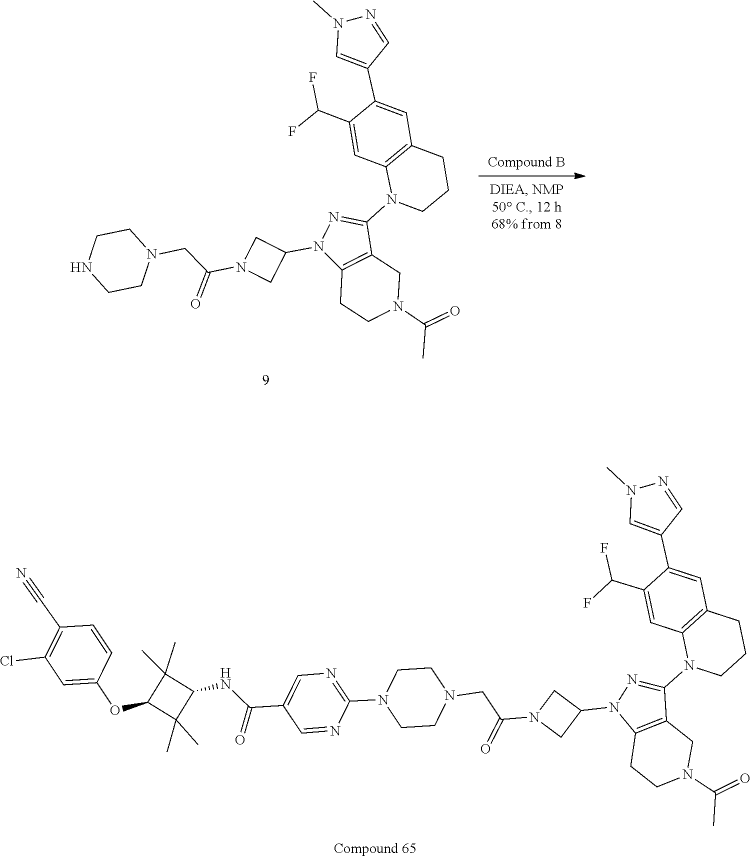 [SMILES: CC(=O)N1CCc2c(c(N3CCCc4cc(-c5cnn(C)c5)c(C(F)F)cc43)nn2C2CN(C(=O)CN3CCNCC3)C2)C1 ; O ; CC(=O)N1CCc2c(c(N3CCCc4cc(-c5cnn(C)c5)c(C(F)F)cc43)nn2C2CN(C(=O)CN3CCN(c4ncc(C(=O)N[C@H]5C(C)(C)[C@H](Oc6ccc(C#N)c(Cl)c6)C5(C)C)cn4)CC3)C2)C1]

3A was prepared according to the procedure described in WO 2022/42707 A1.

To a solution of benzyl 3-hydroxyazetidine-1-carboxylate (5.0 g, 24.13 mmol, 1.0 eq) and Et₃N (3.66 g, 36.19 mmol, 5.04 mL, 1.5 eq) in DCM (80 mL) was added MsCl (3.8 g, 33.17 mmol, 2.57 mL, 1.37 eq) at 0° C. The mixture was stirred at 25° C. for 12 h. The mixture was poured into ice-water (100 mL). The aqueous phase was extracted with DCM (100 mL×2). The combined organic phase was washed with brine (80 mL×2), dried with anhydrous sodium sulfate, filtered, and concentrated under vacuum. The residue was purified by silica gel chromatography (10%-50% ethyl acetate in petroleum ether). Compound benzyl 3-methylsulfonyloxyazetidine-1-carboxylate (6.8 g, 23.83 mmol, 98% yield) was obtained as a colorless oil. LC-MS: MS (ES⁺): t_{R}=0.484 min, m/z=308.0 [M+Na⁺]

To a solution of benzyl 3-methylsulfonyloxyazetidine-1-carboxylate (6.8 g, 23.83 mmol, 2.1 eq) in DMF (60 mL) was added K₂CO₃ (4.75 g, 34.37 mmol, 3.0 eq) and tert-butyl 3-iodo-1,4,6,7-tetrahydropyrazolo[4,3-c]pyridine-5-carboxylate (4.0 g, 11.46 mmol, 1.0 eq). The mixture was stirred at 100° C. for 12 h. The mixture was poured into water (300 mL). The aqueous phase was extracted with ethyl acetate (300 mL×2). The combined organic phase was washed with brine (250 mL×3), dried with anhydrous sodium sulfate, filtered, and concentrated in vacuum. The residue was purified by silica gel chromatography (5-50% ethyl acetate in petroleum ether) and further purified by prep-HPLC (column: Kromasil Eternity XT 250*80 mm*10 um; mobile phase: [water (NH4HCO3)-ACN]; gradient: 50%-80% B over 30 min). Compound tert-butyl 1-(1-benzyloxycarbonylazetidin-3-yl)-3-iodo-6,7-dihydro-4H-pyrazolo[4,3-c]pyridine-5-carboxylate (1.93 g, 3.58 mmol, 31% yield) was obtained as a white solid. LC-MS: MS (ESI⁺): t_{R}=0.627 min, m/z=539.1 [M+H⁺]. ¹H NMR (400 MHz, CDCl₃): δ=7.42-7.31 (m, 5H), 5.14 (s, 2H), 5.00-4.88 (m, 1H), 4.53 (s, 2H), 4.42-4.35 (m, 2H), 4.23 (s, 2H), 3.71 (s, 2H), 2.64 (t, J=5.6 Hz, 2H), 1.51 (s, 9H).

A mixture of tert-butyl 1-(1-benzyloxycarbonylazetidin-3-yl)-3-iodo-6,7-dihydro-4H-pyrazolo[4,3-c]pyridine-5-carboxylate (700 mg, 1.30 mmol, 1.0 eq), 7-(difluoromethyl)-6-(1-methylpyrazol-4-yl)-1,2,3,4-tetrahydroquinoline (479 mg, 1.82 mmol, 1.4 eq), CPHOS PD G3 (105 mg, 130 μmol, 0.1 eq) and Cs₂CO₃ (1.27 g, 3.90 mmol, 3.0 eq) in 2-methylbutan-2-ol (10 mL) was degassed and purged with N₂ for 3 times, and then the mixture was stirred at 90° C. for 12 h under N2 atmosphere. The mixture was concentrated under reduced pressure. The residue was purified by silica gel chromatography (10%-70% ethyl acetate in petroleum ether). Compound tert-butyl 1-(1-benzyloxycarbonylazetidin-3-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridine-5-carboxylate (600 mg, 890 μmol, 68% yield) was obtained as a light yellow solid. LC-MS: MS (ESI⁺): t_{R}=0.611 min, m/z=674.3 [M+H⁺]

To a solution of tert-butyl 1-(1-benzyloxycarbonylazetidin-3-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridine-5-carboxylate (690 mg, 1.02 mmol, 1.0 eq) in DCM (10 mL) was added TFA (7.67 g, 67.31 mmol, 5.00 mL, 65.7 eq). The mixture was stirred at 25° C. for 1 h. The mixture was concentrated under reduced pressure. Compound benzyl 3-[3-[7-(difluoromethyl)-6-(1-methylpyrazol-4-yl)-3,4-dihydro-2H-quinolin-1-yl]-4,5,6,7-tetrahydropyrazolo[4,3-c]pyridin-1-yl]azetidine-1-carboxylate (585 mg) was obtained as a yellow gum and directly used in the next step without further purification. LC-MS: MS (ESI⁺): t_{R}=0.462 min, m/z=574.3 [M+H⁺]

To a solution of benzyl 3-[3-[7-(difluoromethyl)-6-(1-methylpyrazol-4-yl)-3,4-dihydro-2H-quinolin-1-yl]-4,5,6,7-tetrahydropyrazolo[4,3-c]pyridin-1-yl]azetidine-1-carboxylate (585 mg, 1.02 mmol, 1.0 eq) in DCM (15 mL) was added TEA (309 mg, 3.05 mmol, 425 μL, 3.0 eq) and Ac₂O (155 mg, 1.53 mmol, 143 μL, 1.5 eq). The mixture was stirred at 25° C. for 12 h. The mixture was concentrated under reduced pressure. The residue was purified by silica gel chromatography (20%-100% ethyl acetate in petroleum ether). Compound benzyl 3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidine-1-carboxylate (597 mg, 969 μmol, 95% yield over two steps) was obtained as a light yellow solid. LC-MS: MS (ESI⁺): t_{R}=0.528 min, m/z=616.3 [M+H⁺]

To a solution of benzyl 3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidine-1-carboxylate (300 mg, 487 μmol, 1.0 eq) in EtOAc (10 mL) was added Pd/C (100 mg, 10% Pd on carbon, w/w) under N₂ atmosphere. The suspension was degassed and purged with H2 for 3 times. The mixture was stirred under H₂ (15 Psi) at 25° C. for 2 h. The mixture was filtered and concentrated under reduced pressure. The residue was purified by prep-HPLC (column: Welch Xtimate C18 150*25 mm*5 um; mobile phase: [water (FA)-ACN]; gradient: 5%-35% B over 10 min). Compound 1-[1-(azetidin-3-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (130 mg, 234 μmol, 48% yield, FA) was obtained as a light yellow solid. LC-MS: MS (ESI⁺): t_{R}=0.403 min, m/z=482.2 [M+H⁺]. ¹H NMR (400 MHz, CDCl₃): δ=8.53 (s, 1H), 7.55 (d, J=6.0 Hz, 1H), 7.42 (d, J=6.4 Hz, 1H), 7.12-7.02 (m, 1H), 6.90 (d, J=8.4 Hz, 1H), 6.71-6.38 (m, 1H), 5.31-5.12 (m, 1H), 4.59 (t, J=8.0 Hz, 2H), 4.39-4.34 (m, 2H), 4.20 (s, 1H), 4.07 (s, 1H), 3.96 (d, J=2.4 Hz, 3H), 3.88 (t, J=5.6 Hz, 1H), 3.81-3.69 (m, 3H), 2.88 (td, J=6.0, 12.4 Hz, 2H), 2.80-2.63 (m, 2H), 2.17-2.00 (m, 5H).

To a solution of 1-[1-(azetidin-3-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (130 mg, 246 μmol, 1.0 eq, FA) and 2-(4-tert-butoxycarbonylpiperazin-1-yl)acetic acid (66 mg, 271 μmol, 1.1 eq) in DMF (3 mL) was added HATU (140 mg, 369 μmol, 1.5 eq) and DIEA (95 mg, 739 μmol, 128 μL, 3.0 eq). The mixture was stirred at 25° C. for 12 h. The mixture was poured into water (40 mL). The aqueous phase was extracted with ethyl acetate (40 mL×2). The combined organic phase was washed with brine (40 mL×2), dried with anhydrous sodium sulfate, filtered and concentrated in vacuum. The residue was purified by prep-Thin-layer chromatography (silica, dichloromethane:methanol=10:1). Compound tert-butyl 4-[2-[3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidin-1-yl]-2-oxo-ethyl]piperazine-1-carboxylate (130 mg, 183 μmol, 75% yield) was obtained as a yellow solid. LC-MS: MS (ESI⁺): t_{R}=0.446 min, m/z=708.4 [M+H⁺]

To a solution of tert-butyl 4-[2-[3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidin-1-yl]-2-oxo-ethyl]piperazine-1-carboxylate (130 mg, 183 mol, 1.0 eq) in DCM (6 mL) was added TFA (3.07 g, 26.92 mmol, 2.0 mL, 146.6 eq). The mixture was stirred at 25° C. for 1 h. The mixture was concentrated under reduced pressure. Compound 1-[3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidin-1-yl]-2-piperazin-1-yl-ethanone (110 mg) was obtained as a yellow gum and directly used in the next step without further purification. LC-MS: MS (ESI⁺): t_{R}=0.405 min, m/z=608.4 [M+H⁺]

To a solution of 1-[3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidin-1-yl]-2-piperazin-1-yl-ethanone (110 mg) in NMP (2 mL) was added DIEA (93 mg, 720 μmol, 125 μL, 4.0 eq) and 2-chloro-N-[3-(3-chloro-4-cyano-phenoxy)-2,2,4,4-tetramethyl-cyclobutyl]pyrimidine-5-carboxamide (76 mg, 180 μmol, 1.0 eq). The mixture was stirred at 50° C. for 12 h. The mixture was filtered. The residue was purified by prep-HPLC (column: Welch Xtimate C18 150*25 mm*5 um; mobile phase: [water (FA)-ACN]; gradient: 25%-55% B over 15 min). Compound 2-[4-[2-[3-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]azetidin-1-yl]-2-oxo-ethyl]piperazin-1-yl]-N-[3-(3-chloro-4-cyano-phenoxy)-2,2,4,4-tetramethyl-cyclobutyl]pyrimidine-5-carboxamide (Compound 65, 123 mg, 122 μmol, 68% yield over two steps) was obtained as an off-white solid. ¹H NMR (400 MHz, CDCl₃): δ=8.71 (s, 2H), 7.59 (d, J=8.8 Hz, 1H), 7.57-7.50 (m, 1H), 7.46-7.38 (m, 1H), 7.11-7.03 (m, 1H), 6.98 (d, J=2.4 Hz, 1H), 6.92 (d, J=6.0 Hz, 1H), 6.82 (dd, J=2.4, 8.8 Hz, 1H), 6.71-6.38 (m, 1H), 5.98 (d, J=8.0 Hz, 1H), 5.04-4.93 (m, 1H), 4.89-4.79 (m, 1H), 4.75-4.65 (m, 1H), 4.58-4.44 (m, 2H), 4.35-4.18 (m, 1H), 4.18-4.10 (m, 2H), 4.08-4.06 (m, 1H), 4.06-3.89 (m, 8H), 3.89-3.72 (m, 3H), 3.17 (d, J=1.2 Hz, 2H), 2.95-2.86 (m, 2H), 2.82-2.70 (m, 2H), 2.62 (t, J=4.8 Hz, 4H), 2.20-2.03 (m, 5H), 1.25 (d, J=13.6 Hz, 12H). LC-MS: MS (ESI⁺): t_{R}=2.276 min, m/z=990.5 [M+H⁺]

### Example 12: Synthesis of 2-[4-[[[5-[8-(5-acetyl-1-tetrahydropyran-4-yl-6,7-dihydro-4H-pyrazolo[4,3-c]pyridin-3-yl)-3-isoquinolyl]pyridine-2-carbonyl]amino]methyl]-1-piperidyl]-N-[3-(3-chloro-4-cyano-phenoxy)-2,2,4,4-tetramethyl-cyclobutyl]pyrimidine-5-carboxamide (Compound 110)

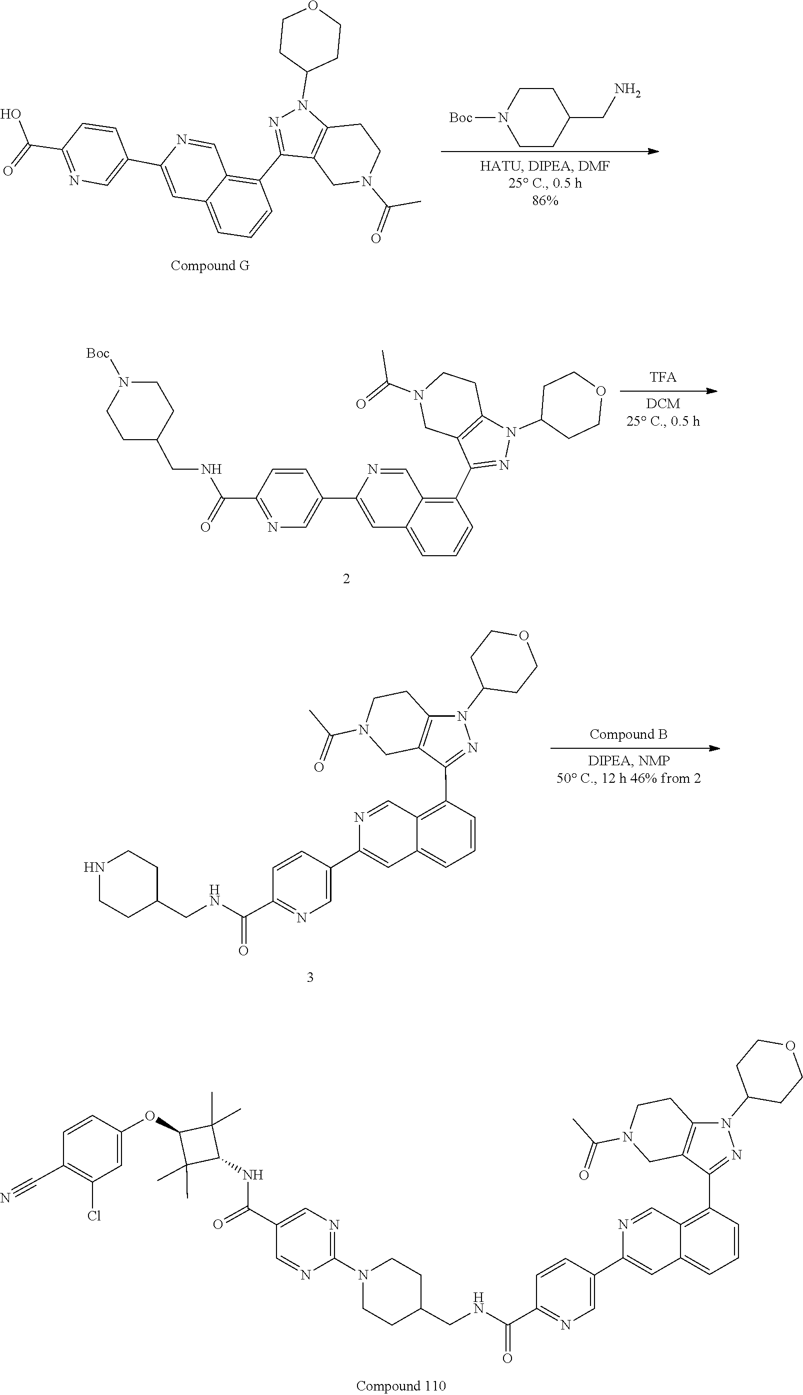 [SMILES: CC(=O)N1CCc2c(c(-c3cccc4cc(-c5ccc(C(=O)O)nc5)ncc34)nn2C2CCOCC2)C1 ; CC(C)(C)OC(=O)N1CCC(CN)CC1 ; CC(=O)N1CCc2c(c(-c3cccc4cc(-c5ccc(C(=O)NCC6CCN(C(=O)OC(C)(C)C)CC6)nc5)ncc34)nn2C2CCOCC2)C1 ; CC(=O)N1CCc2c(c(-c3cccc4cc(-c5ccc(C(=O)NCC6CCNCC6)nc5)ncc34)nn2C2CCOCC2)C1 ; CC(=O)N1CCc2c(c(-c3cccc4cc(-c5ccc(C(=O)NCC6CCN(c7ncc(C(=O)N[C@H]8C(C)(C)[C@H](Oc9ccc(C#N)c(Cl)c9)C8(C)C)cn7)CC6)nc5)ncc34)nn2C2CCOCC2)C1]

To a solution of 5-[8-(5-acetyl-1-tetrahydropyran-4-yl-6,7-dihydro-4H-pyrazolo[4,3-c]pyridin-3-yl)-3-isoquinolyl]pyridine-2-carboxylic acid (16.7 mg, 33.7 μmol, 1.0 eq) tert-butyl 4-(aminomethyl)piperidine-1-carboxylate (7.2 mg, 33.7 μmol, 1.0 eq) and DIPEA (13 mg, 101.12 μmol, 17 μL, 3.0 eq) in DMF (1 mL) was added HATU (25 mg, 67.42 μmol, 2.0 eq). The mixture was stirred at 25° C. for 0.5 h. The mixture was concentrated under reduced pressure. The residue was purified by prep-TLC (silica, ethyl acetate). Compound tert-butyl 4-[[[5-[8-(5-acetyl-1-tetrahydropyran-4-yl-6,7-dihydro-4H-pyrazolo[4,3-c]pyridin-3-yl)-3-isoquinolyl]pyridine-2-carbonyl]amino]methyl]piperidine-1-carboxylate (20 mg, 28.8 μmol, 86% yield) was obtained as a yellow solid. LC-MS: MS (ESI⁺): t_{R}=0.535 min, m/z=694.3 [M+H⁺]

To a solution of tert-butyl 4-[[[5-[8-(5-acetyl-1-tetrahydropyran-4-yl-6,7-dihydro-4H-pyrazolo[4,3-c]pyridin-3-yl)-3-isoquinolyl]pyridine-2-carbonyl]amino]methyl]piperidine-1-carboxylate (20 mg, 28.83 μmol, 1.0 eq) in DCM (1 mL) was added TFA (767 mg, 6.73 mmol, 0.5 mL, 233.5 eq). The mixture was stirred at 25° C. for 0.5 h. The mixture was concentrated in vacuum. Compound 5-[8-(5-acetyl-1-tetrahydropyran-4-yl-6,7-dihydro-4H-pyrazolo[4,3-c]pyridin-3-yl)-3-isoquinolyl]-N-(4-piperidylmethyl)pyridine-2-carboxamide (17 mg) was obtained as a yellow solid and directly used in the next step without further purification. LC-MS: MS (ESI⁺): t_{R}=0.352 min, m/z=233.0 [M+H⁺]

To a solution of 5-[8-(5-acetyl-1-tetrahydropyran-4-yl-6,7-dihydro-4H-pyrazolo[4,3-c]pyridin-3-yl)-3-isoquinolyl]-N-(4-piperidylmethyl)pyridine-2-carboxamide (17 mg) and 2-chloro-N-[3-(3-chloro-4-cyano-phenoxy)-2,2,4,4-tetramethyl-cyclobutyl]pyrimidine-5-carboxamide (10 mg, 24.02 μmol, 1.0 eq) in NMP (1 mL) was added DIPEA (9 mg, 72.06 μmol, 12 μL, 3.0 eq). The mixture was stirred at 50° C. for 12 h and then concentrated under reduced pressure. The residue was purified by prep-HPLC (silica, column: YMC-Actus Triart C18 150*30 mm*7 um; mobile phase: [water(FA)-ACN]; gradient: 65%-95% B over 10 min). Compound 2-[4-[[[5-[8-(5-acetyl-1-tetrahydropyran-4-yl-6,7-dihydro-4H-pyrazolo[4,3-c]pyridin-3-yl)-3-isoquinolyl]pyridine-2-carbonyl]amino]methyl]-1-piperidyl]-N-[3-(3-chloro-4-cyano-phenoxy)-2,2,4,4-tetramethyl-cyclobutyl]pyrimidine-5-carboxamide (Compound 110, 10 mg, 10.9 μmol, 46% yield over two steps) was obtained as a white solid. ¹H NMR (400 MHz, CDCl₃): δ 9.88 (d, J=2.8 Hz, 1H) 9.27-9.34 (m, 1H) 8.70 (s, 2H) 8.57-8.64 (m, 1H) 8.33 (dd, J=8.4, 3.6 Hz, 1H) 8.28 (br t, J=5.2 Hz, 1H) 8.19 (d, J=10.4 Hz, 1H) 7.93 (dd, J=13.6, 8.4 Hz, 1H) 7.80 (dt, J=12.0, 7.6 Hz, 1H) 7.63 (dd, J=17.2, 6.4 Hz, 1H) 7.58 (d, J=8.8 Hz, 1H) 6.97 (d, J=2.4 Hz, 1H) 6.81 (dd, J=8.8, 2.4 Hz, 1H) 5.92 (d, J=8.0 Hz, 1H) 4.88-4.97 (m, 2H) 4.68 (s, 1H) 4.52 (s, 1H) 4.24-4.36 (m, 1H) 4.16-4.23 (m, 2H) 4.13 (d, J=8.0 Hz, 1H) 4.01-4.06 (m, 2H) 3.87 (t, J=5.6 Hz, 1H) 3.53-3.66 (m, 2H) 3.47 (t, J=6.4 Hz, 2H) 2.92-3.06 (m, 3H) 2.88 (br t, J=5.6 Hz, 1H) 2.39-2.55 (m, 2H) 2.23 (s, 1H) 2.09 (s, 2H) 1.93-2.01 (m, 4H) 1.30-1.40 (m, 3H) 1.25 (s, 6H) 1.22 (s, 6H). LC-MS: MS (ESI⁺): t_{R}=2.540 min, m/z=976.5 [M+H⁺]

### Example 13: Synthesis of 2-[[5-[8-(5-acetyl-1-tetrahydropyran-4-yl-6,7-dihydro-4H-pyrazolo[4,3-c]pyridin-3-yl)-3-isoquinolyl]-2-pyridyl]methoxy]-N-[3-(3-chloro-4-cyano-phenoxy)-2,2,4,4-tetramethyl-cyclobutyl]pyrimidine-5-carboxamide (Compound 71)

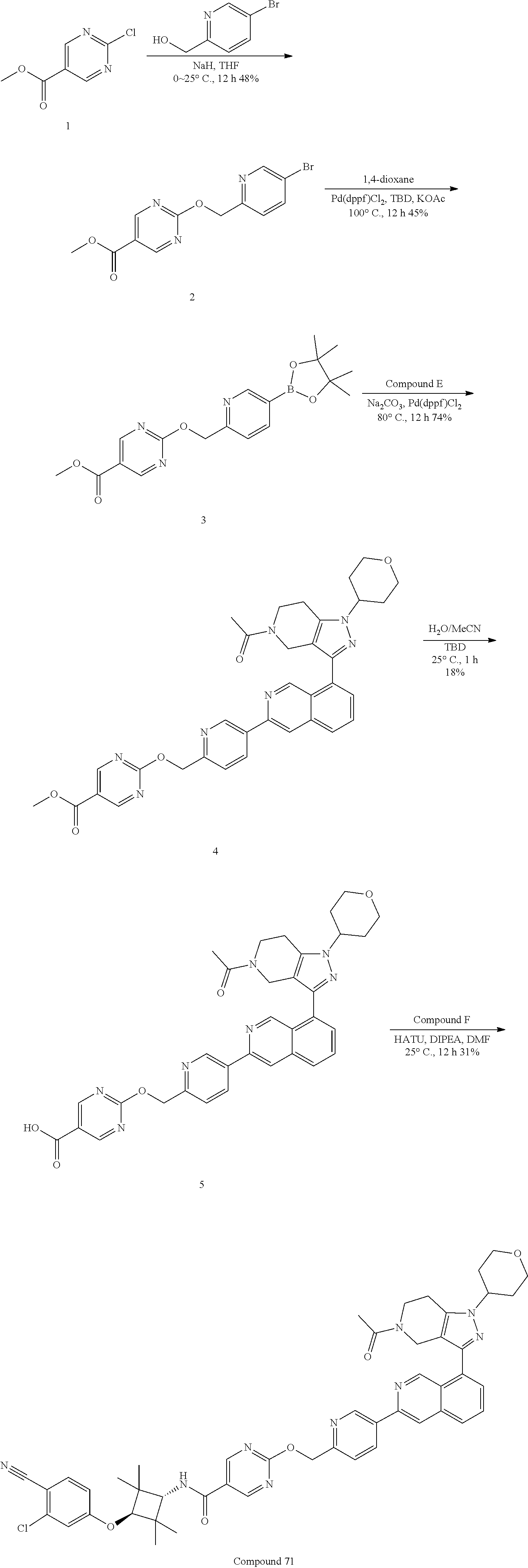

To a solution of (5-bromo-2-pyridyl)methanol (3.27 g, 17 mmol, 1 eq) in THF (30 mL) was added NaH (834.37 mg, 60% w/w, 20 mmol, 1.2 eq) at 0° C. and stirred for 1 h. Then the mixture was added methyl 2-chloropyrimidine-5-carboxylate (3 g, 17 mmol, 1 eq) in THF (10 mL) at 0° C. and stirred at 25° C. for 12 h. The mixture was quenched by saturated NH₄Cl (20 mL). The mixture was extracted with ethyl acetate (20 mL×3). The combined organic phase was washed with brine (20 mL), dried over anhydrous Na₂SO₄, filtered, and concentrated under reduced pressure. The residue was purified by column chromatography (silica, 10˜25% ethyl acetate in petroleum ether). Compound methyl 2-[(5-bromo-2-pyridyl)methoxy]pyrimidine-5-carboxylate (3.0 g, 8 mmol, 48% yield, 90% purity) was obtained as a white solid. LC-MS: MS (ESI⁺): t_{R}=0.495 min, m/z=325.9 [M+H⁺]. ¹H NMR (400 MHz, DMSO-d₆): δ=9.23-8.99 (m, 2H), 8.69 (s, 1H), 8.09 (br dd, J=1.7, 8.2 Hz, 1H), 7.47 (br d, J=8.4 Hz, 1H), 5.54 (s, 2H), 3.87 (s, 3H)

To a solution of methyl 2-[(5-bromo-2-pyridyl)methoxy]pyrimidine-5-carboxylate (1.80 g, 5 mmol, 1 eq) and 4,4,5,5-tetramethyl-2-(4,4,5,5-tetramethyl-1,3,2-dioxaborolan-2-yl)-1,3,2-dioxaborolane (1.41 g, 5 mmol, 1 eq) in 1,4-dioxane (50 mL) was added KOAc (1.64 g, 16 mmol, 3 eq) and Pd(dppf)Cl₂ (81.27 mg, 111 μmol, 0.02 eq). The mixture was stirred at 100° C. for 12 h. The mixture was added water (20 mL) and the mixture was extracted with DCM (20 mL×3). The combined organic phase was washed with brine (20 mL), dried over anhydrous sodium sulfate, filtered and concentrated in vacuo. The residue was purified by column chromatography (silica, 90% ethyl acetate in petroleum ether to 10% MeOH in DCM). Product methyl 2-[[5-(4,4,5,5-tetramethyl-1,3,2-dioxaborolan-2-yl)-2-pyridyl]methoxy]pyrimidine-5-carboxylate (1 g, 2.51 mmol, 45.12% yield) was obtained as a yellow solid. LC-MS: MS (ESI⁺): t_{R}=0.391 min, m/z=372.1 [M+H⁺]. ¹H NMR (400 MHz, CHLOROFORM-d): δ=9.13-9.08 (m, 2H), 8.93 (s, 1H), 8.09 (dd, J=1.6, 7.8 Hz, 1H), 7.50 (d, J=7.8 Hz, 1H), 7.27 (s, 1H), 5.73-5.66 (m, 2H), 3.96-3.93 (m, 3H), 1.36 (s, 12H)

To a solution of [8-(5-acetyl-1-tetrahydropyran-4-yl-6,7-dihydro-4H-pyrazolo[4,3-c]pyridin-3-yl)-3-isoquinolyl]trifluoromethanesulfonate (100 mg, 190 μmol, 1 eq) and methyl 2-[[5-(4,4,5,5-tetramethyl-1,3,2-dioxaborolan-2-yl)-2-pyridyl]methoxy]pyrimidine-5-carboxylate (84.92 mg, 228 μmol, 1.2 eq) in 1,4-dioxane (5 mL) and H₂O (0.5 mL) was added Na₂CO₃ (60.62 mg, 571 μmol, 3 eq) and Pd(dppf)Cl₂ (7.78 mg, 9 μmol, 0.05 eq). The mixture was stirred at 80° C. for 12 h under N₂. The mixture was added water (20 mL) and the mixture was extracted with ethyl acetate (20 mL×3). The combined organic phase was washed with brine (20 mL), dried over anhydrous sodium sulfate, filtered and concentrated in vacuo. The residue was purified by flash silica gel chromatography (10% methanol in dichloromethane). Product methyl 2-[[5-[8-(5-acetyl-1-tetrahydropyran-4-yl-6,7-dihydro-4H-pyrazolo[4,3-c]pyridin-3-yl)-3-isoquinolyl]-2-pyridyl]methoxy]pyrimidine-5-carboxylate (120 mg, 141.37 μmol, 74.15% yield, 73% purity) was obtained as a yellow solid. LC-MS: MS (ESI⁺): t_{R}=0.498 min, m/z=620.3 [M+H⁺]

To a solution of methyl 2-[[5-[8-(5-acetyl-1-tetrahydropyran-4-yl-6,7-dihydro-4H-pyrazolo[4,3-c]pyridin-3-yl)-3-isoquinolyl]-2-pyridyl]methoxy]pyrimidine-5-carboxylate (120 mg, 141 μmol, 1 eq) in MeCN (12 mL) and H₂O (4 mL) was added 3,4,6,7,8,9-hexahydro-2H-pyrimido[1,2-a]pyrimidine (59.03 mg, 424 μmol, 3 eq). The mixture was stirred at 25° C. for 1 h. The mixture was added H₂O (20 mL) and the mixture was extracted with ethyl acetate (20 mL×3). The combined organics were washed with brine (20 mL), dried over anhydrous Na₂SO₄, filtered, and concentrated in vacuo. The residue was purified by semi-preparative reverse-phase HPLC (column: Phenomenex luna C18 150*25 mm*10 um; mobile phase: [water(FA)-ACN]; gradient: 20%-50% B over 1 min). Product 2-[[5-[8-(5-acetyl-1-tetrahydropyran-4-yl-6,7-dihydro-4H-pyrazolo[4,3-c]pyridin-3-yl)-3-isoquinolyl]-2-pyridyl]methoxy]pyrimidine-5-carboxylic acid (15 mg, 24 μmol, 17.52% yield) was obtained as a yellow solid. LC-MS: MS (ESI⁺): t_{R}=0.462 min, m/z=606.3 [M+H⁺]

To a solution of 2-[[5-[8-(5-acetyl-1-tetrahydropyran-4-yl-6,7-dihydro-4H-pyrazolo[4,3-c]pyridin-3-yl)-3-isoquinolyl]-2-pyridyl]methoxy]pyrimidine-5-carboxylic acid (30 mg, 49 μmol, 1 eq) and 4-(3-amino-2,2,4,4-tetramethyl-cyclobutoxy)-2-chloro-benzonitrile (13.81 mg, 49 μmol, 1 eq) in DMF (2 mL) was added DIPEA (32.01 mg, 247 μmol, 43.14 μL, 5 eq) and HATU (94.17 mg, 247 μmol, 5 eq). The mixture was stirred at 25° C. for 12 h. The reaction mixture was concentrated in vacuo to give the crude product. The residue was purified by semi-preparative reverse phase HPLC (column: Welch Xtimate C18 150*25 mm*5 um; mobile phase: [water(FA)-ACN]; gradient: 50%-80% B over 15 min). Product 2-[[5-[8-(5-acetyl-1-tetrahydropyran-4-yl-6,7-dihydro-4H-pyrazolo[4,3-c]pyridin-3-yl)-3-isoquinolyl]-2-pyridyl]methoxy]-N-[3-(3-chloro-4-cyano-phenoxy)-2,2,4,4-tetramethyl-cyclobutyl]pyrimidine-5-carboxamide (Compound 71, 13.62 mg, 15 μmol, 31.13% yield) was obtained as a yellow solid. ¹H NMR (400 MHz, DMSO-d₆): δ=9.97 (s, 1H), 9.41 (d, J=2.0 Hz, 1H), 9.01 (s, 2H), 8.67-8.57 (m, 2H), 8.14-8.02 (m, 2H), 7.94-7.84 (m, 2H), 7.74-7.59 (m, 2H), 7.22 (d, J=2.4 Hz, 1H), 7.01 (dd, J=2.5, 8.8 Hz, 1H), 5.65 (s, 2H), 4.57 (br s, 2H), 4.48 (br d, J=4.3 Hz, 1H), 4.29 (s, 1H), 4.10-3.96 (m, 3H), 3.90-3.77 (m, 2H), 3.53 (br t, J=11.8 Hz, 2H), 3.00-2.82 (m, 2H), 2.11 (s, 4H), 2.02 (s, 1H), 2.00-1.92 (m, 2H), 1.23 (s, 6H), 1.12 (s, 6H). LC-MS: MS (ESI⁺): t_{R}=2.232 min, m/z=866.6 [M+H⁺]

### Example 14: Synthesis of 2-[2-[[5-[8-(5-acetyl-1-tetrahydropyran-4-yl-6,7-dihydro-4H-pyrazolo[4,3-c]pyridin-3-yl)-3-isoquinolyl]pyridine-2-carbonyl]amino]ethoxymethyl]-N-[3-(3-chloro-4-cyano-phenoxy)-2,2,4,4-tetramethyl-cyclobutyl]pyrimidine-5-carboxamide (Compound 76)

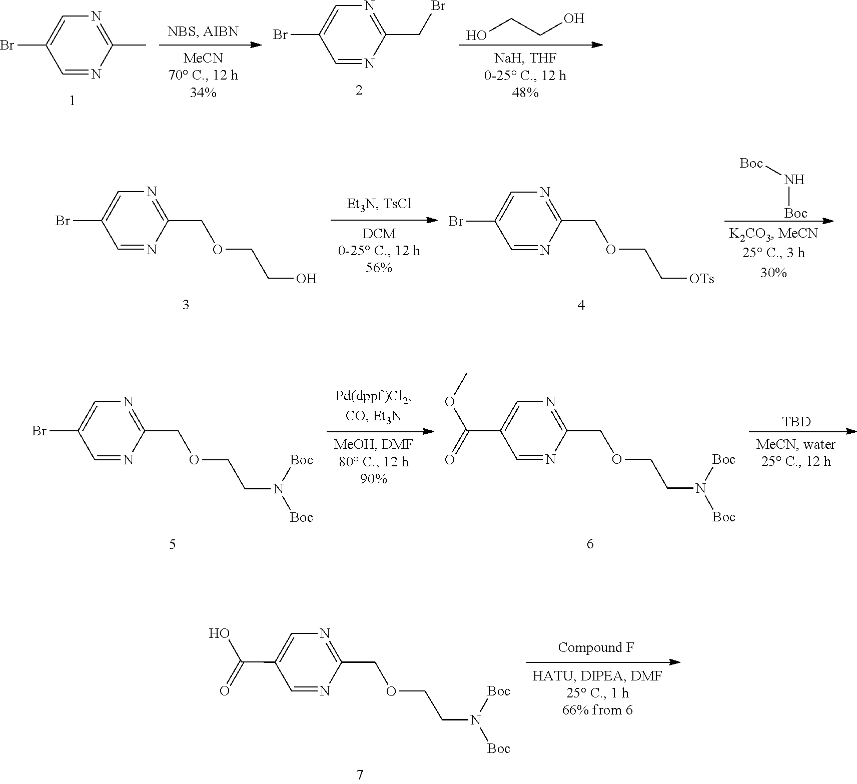

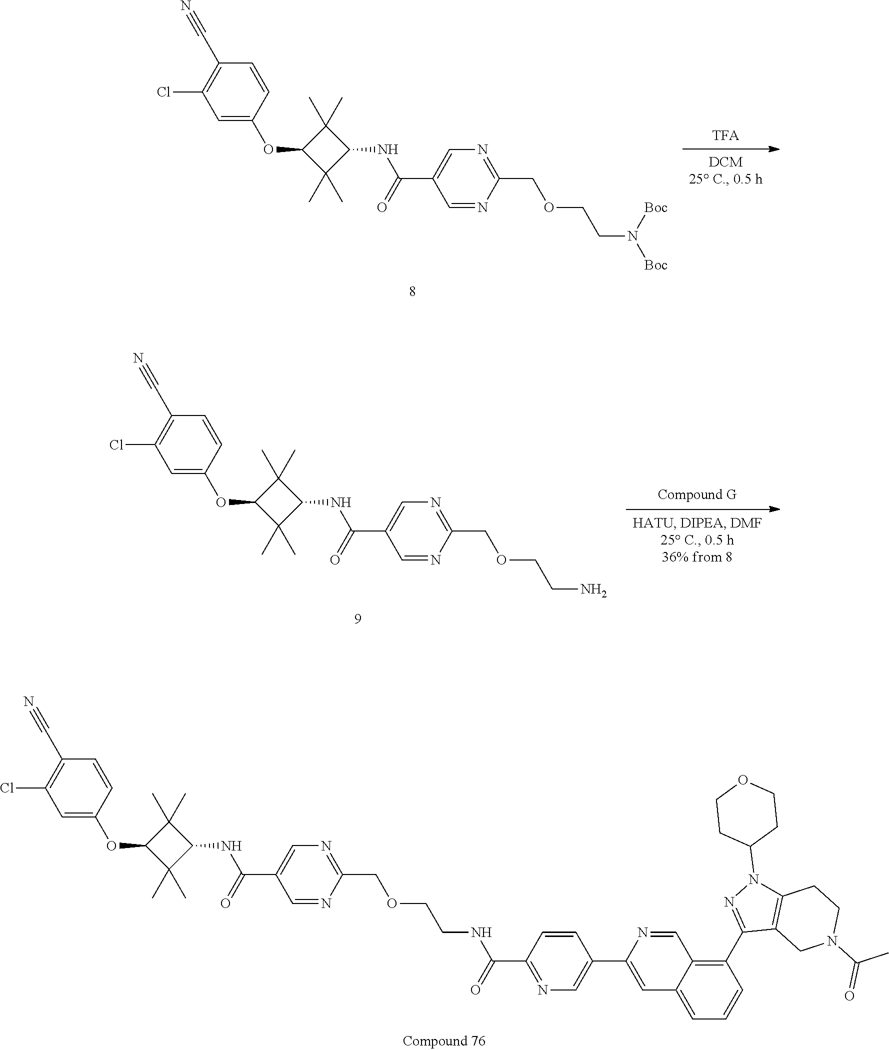 [SMILES: CC(C)(C)OC(=O)N(CCOCc1ncc(C(=O)N[C@H]2C(C)(C)[C@H](Oc3ccc(C#N)c(Cl)c3)C2(C)C)cn1)C(=O)OC(C)(C)C ; CC1(C)[C@H](NC(=O)c2cnc(COCCN)nc2)C(C)(C)[C@H]1Oc1ccc(C#N)c(Cl)c1 ; CC(=O)N1CCc2c(c(-c3cccc4cc(-c5ccc(C(=O)NCCOCc6ncc(C(=O)N[C@H]7C(C)(C)[C@H](Oc8ccc(C#N)c(Cl)c8)C7(C)C)cn6)nc5)ncc34)nn2C2CCOCC2)C1]

To a solution of 5-bromo-2-methyl-pyrimidine (10 g, 57.80 mmol, 1 eq) and NBS (30.86 g, 173.40 mmol, 3 eq) in MeCN (100 mL) was added AIBN (3.32 g, 20.23 mmol, 0.35 eq). The mixture was stirred at 80° C. for 12 h. The mixture concentrated under reduced pressure. The residue was purified by column chromatography (silica, petroleum ether/ethyl acetate=50/1 to 10/1). Compound 5-bromo-2-(bromomethyl)pyrimidine (5.0 g, 19.85 mmol, 34% yield) was obtained as a yellow solid. LC-MS: MS (ESI⁺): t_{R}=0.443 min, m/z=252.7 [M+H⁺]

To a solution of 5-bromo-2-(bromomethyl)pyrimidine (1 g, 3.97 mmol, 1.0 eq) and NaH (220 mg, 60% w/w, 5.9 mmol, 1.5 eq) in THF (10 mL) was added ethylene glycol (493 mg, 7.94 mmol, 442.75 μL, 2.0 eq) at 0° C. The mixture was stirred at 0-25° C. for 12 h under N₂ atmosphere. The reaction mixture was quenched with saturated aqueous NH₄Cl (100 mL) at 0° C. dropwise. The resulting mixture was extracted with ethyl acetate (100 mL×3). The combined organics were dried over anhydrous Na₂SO₄, filtered, and concentrated in vacuum. The residue was purified by column chromatography (silica, petroleum ether/ethyl acetate=50/1 to 10/1). Compound 2-[(5-bromopyrimidin-2-yl)methoxy]ethanol (0.45 g, 1.93 mmol, 48% yield) was obtained as a colorless oil. LC-MS: MS (ESI⁺): t_{R}=0.352 min, m/z=233.0 [M+H⁺]

To a solution of 2-[(5-bromopyrimidin-2-yl)methoxy]ethanol (0.45 g, 1.93 mmol, 1 eq) and Et₃N (586.13 mg, 5.79 mmol, 806.23 μL, 3 eq) in DCM (5 mL) was added TosCl (375.47 mg, 1.97 mmol, 1.02 eq) at 0° C. The mixture was stirred at 0-25° C. for 12 h. The mixture concentrated in vacuum. The residue was purified by column chromatography (silica, petroleum ether/ethyl acetate=50/1 to 10/1). Compound 2-[(5-bromopyrimidin-2-yl)methoxy]ethyl 4-methylbenzenesulfonate (0.42 g, 1.08 mmol, 56% yield) was obtained as a yellow solid. LC-MS: MS (ESI⁺): t_{R}=0.527 min, m/z=409.0 [M+Na⁺]

To a solution of 2-[(5-bromopyrimidin-2-yl)methoxy]ethyl 4-methylbenzenesulfonate (0.42 g, 1.08 mmol, 1.0 eq) and Cs₂CO₃ (530.06 mg, 1.63 mmol, 1.5 eq) in DMF (5 mL) was added tert-butyl N-tert-butoxycarbonylcarbamate (235.64 mg, 1.08 mmol, 1.0 eq). The mixture was stirred at 25° C. for 3 h. The mixture was poured into iced water (100 mL) and extracted with ethyl acetate (100 mL×2). The combined organic phase was washed with brine (100 mL×2), dried with anhydrous sodium sulfate, filtered and concentrated in vacuum. The residue was purified by column chromatography (silica, petroleum ether/ethyl acetate=50/1 to 10/1). Compound tert-butyl N-[2-[(5-bromopyrimidin-2-yl)methoxy]ethyl]-N-tert-butoxycarbonyl-carbamate (0.14 g, 323.84 mol, 30% yield) was obtained as a yellow solid. LC-MS: MS (ESI⁺): t_{R}=0.643 min, m/z=887.3 [2M+Na⁺]

To a solution of tert-butyl N-[2-[(5-bromopyrimidin-2-yl)methoxy]ethyl]-N-tert-butoxycarbonyl-carbamate (0.14 g, 323.8 mol, 1.0 eq), Et₃N (98.3 mg, 971.5 μmol, 135.2 μL, 3.0 eq) in MeOH (2 mL) and DMF (2 mL) was added Pd(dppf)Cl₂ (23.70 mg, 32.38 μmol, 0.1 eq). The mixture was stirred at 80° C. for 12 h under carbon monoxide atmosphere. The mixture was poured into iced water (100 mL) and extracted with ethyl acetate (100 mL×2). The combined organic phase was washed with brine (100 mL×2), dried with anhydrous Na₂SO₄, filtered, and concentrated under reduced pressure. The residue was purified by column chromatography (silica, petroleum ether/ethyl acetate=50/1 to 5/1). Compound methyl 2-[2-[bis(tert-butoxycarbonyl)amino]ethoxymethyl]pyrimidine-5-carboxylate (0.12 g, 291.65 μmol, 90% yield) was obtained as a yellow solid. LC-MS: MS (ESI⁺): t_{R}=0.552 min, m/z=845.4 [2M+Na⁺]

To a solution of methyl 2-[2-[bis(tert-butoxycarbonyl)amino]ethoxymethyl]pyrimidine-5-carboxylate (0.12 g, 292 mol, 1.0 eq) in MeCN (3 mL) and H₂O (1 mL) was added TBD (81.2 mg, 583 μmol, 2.0 eq). The mixture was stirred at 25° C. for 12 h. The mixture was added 1 M HCl (50 μL) and extracted with DCM (100 mL×2). The combined organics were washed with brine (100 mL×2), dried with anhydrous Na₂SO₄, filtered, and concentrated under reduced pressure. Compound 2-[2-[bis(tert-butoxycarbonyl)amino]ethoxymethyl]pyrimidine-5-carboxylic acid (0.11 g) was obtained as a white solid and directly used in the next step without further purification. LC-MS: MS (ESI⁺): t_{R}=0.518 min, m/z=817.4 [2M+Na⁺]

To a solution of 2-[2-[bis(tert-butoxycarbonyl)amino]ethoxymethyl]pyrimidine-5-carboxylic acid (0.11 g), 4-(3-amino-2,2,4,4-tetramethyl-cyclobutoxy)-2-chloro-benzonitrile (70 mg, 252 μmol, 1.0 eq) and DIPEA (98 mg, 752 μmol, 131 μL, 3.0 eq) in DMF (2 mL) was added HATU (191 mg, 503 μmol, 2.0 eq). The mixture was stirred at 25° C. for 1 h. The mixture was poured into iced water (100 mL) and extracted with DCM (100 mL×2). The combined organics were washed with brine (100 mL×2), dried with anhydrous Na₂SO₄, filtered, and concentrated under reduced pressure. The residue was purified by column chromatography (silica, petroleum ether/ethyl acetate=50/1 to 2/1). Compound tert-butyl N-tert-butoxycarbonyl-N-[2-[[5-[[3-(3-chloro-4-cyano-phenoxy)-2,2,4,4-tetramethyl-cyclobutyl]carbamoyl]pyrimidin-2-yl]methoxy]ethyl]carbamate (0.11 g, 167.1 μmol, 66% yield over two steps) was obtained as a white solid. LC-MS: MS (ESI⁺): t_{R}=0.668 min, m/z=680.3 [M+Na⁺]

To a solution of tert-butyl N-tert-butoxycarbonyl-N-[2-[[5-[[3-(3-chloro-4-cyano-phenoxy)-2,2,4,4-tetramethyl-cyclobutyl]carbamoyl]pyrimidin-2-yl]methoxy]ethyl]carbamate (55 mg, 83.56 μmol, 1.0 eq) in DCM (1 mL) was added TFA (844 mg, 7.40 mmol, 550 μL, 88.6 eq). The mixture was stirred at 25° C. for 0.5 h. The mixture was concentrated in vacuum. Compound 2-(2-aminoethoxymethyl)-N-[3-(3-chloro-4-cyano-phenoxy)-2,2,4,4-tetramethyl-cyclobutyl]pyrimidine-5-carboxamide (36 mg) was obtained as a yellow solid and directly used in the next step without further purification.

To a solution of 2-(2-aminoethoxymethyl)-N-[3-(3-chloro-4-cyano-phenoxy)-2,2,4,4-tetramethyl-cyclobutyl]pyrimidine-5-carboxamide (36 mg) 5-[8-(5-acetyl-1-tetrahydropyran-4-yl-6,7-dihydro-4H-pyrazolo[4,3-c]pyridin-3-yl)-3-isoquinolyl]pyridine-2-carboxylic acid (33.6 mg, 67.5 μmol, 1.1 eq) and DIPEA (24.4 mg, 189 μmol, 32.9 μL, 3.0 eq) in DMF (1 mL) was added HATU (47.9 mg, 126 μmol, 2.0 eq). The mixture was stirred at 25° C. for 0.5 h. The mixture was concentrated under reduced pressure. The residue was purified by prep-HPLC (column: YMC-Actus Triart C18 150*30 mm*7 um; mobile phase: [water(FA)-ACN]; gradient: 53%-83% B over 10 min). Compound 2-[2-[[5-[8-(5-acetyl-1-tetrahydropyran-4-yl-6,7-dihydro-4H-pyrazolo[4,3-c]pyridin-3-yl)-3-isoquinolyl]pyridine-2-carbonyl]amino]ethoxymethyl]-N-[3-(3-chloro-4-cyano-phenoxy)-2,2,4,4-tetramethyl-cyclobutyl]pyrimidine-5-carboxamide (Compound 76, 21.82 mg, 22.81 μmol, 36% yield over two steps) was obtained as a yellow solid. ¹H NMR (400 MHz, CDCl₃): δ 9.90 (d, J=13.2 Hz, 1H) 9.31-9.39 (m, 1H) 9.16 (s, 2H) 8.85-9.01 (m, 1H) 8.61 (dd, J=8.0, 2.0 Hz, 1H) 8.34 (d, J=8.0 Hz, 1H) 8.21 (d, J=9.6 Hz, 1H) 7.93 (dd, J=14.8, 7.6 Hz, 1H) 7.80 (dt, J=11.6, 7.6 Hz, 1H) 7.62 (dd, J=15.6, 7.2 Hz, 1H) 7.45-7.56 (m, 1H) 6.93 (dd, J=8.0, 2.4 Hz, 1H) 6.73 (td, J=8.8, 2.0 Hz, 1H) 6.17-6.25 (m, 1H) 4.94 (s, 2H) 4.67 (s, 1H) 4.51 (s, 1H) 4.23-4.37 (m, 1H) 4.10-4.22 (m, 3H) 3.98-4.07 (m, 2H) 3.90-3.96 (m, 2H) 3.80-3.89 (m, 3H) 3.54-3.63 (m, 2H) 2.79-3.02 (m, 2H) 2.32-2.53 (m, 2H) 2.05-2.30 (m, 3H) 1.97 (br d, J=11.6 Hz, 2H) 1.25 (s, 3H) 1.23 (s, 3H) 1.22 (s, 3H) 1.20 (s, 3H). LC-MS: MS (ESI⁺): t_{R}=2.273 min, m/z=937.6 [M+H⁺]

### Example 15: Synthesis of 1-[1-[2-[5-[[3-(3-chloro-4-cyano-phenoxy)-2,2,4,4-tetramethyl-cyclobutyl]carbamoyl]pyrimidin-2-yl]ethyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound

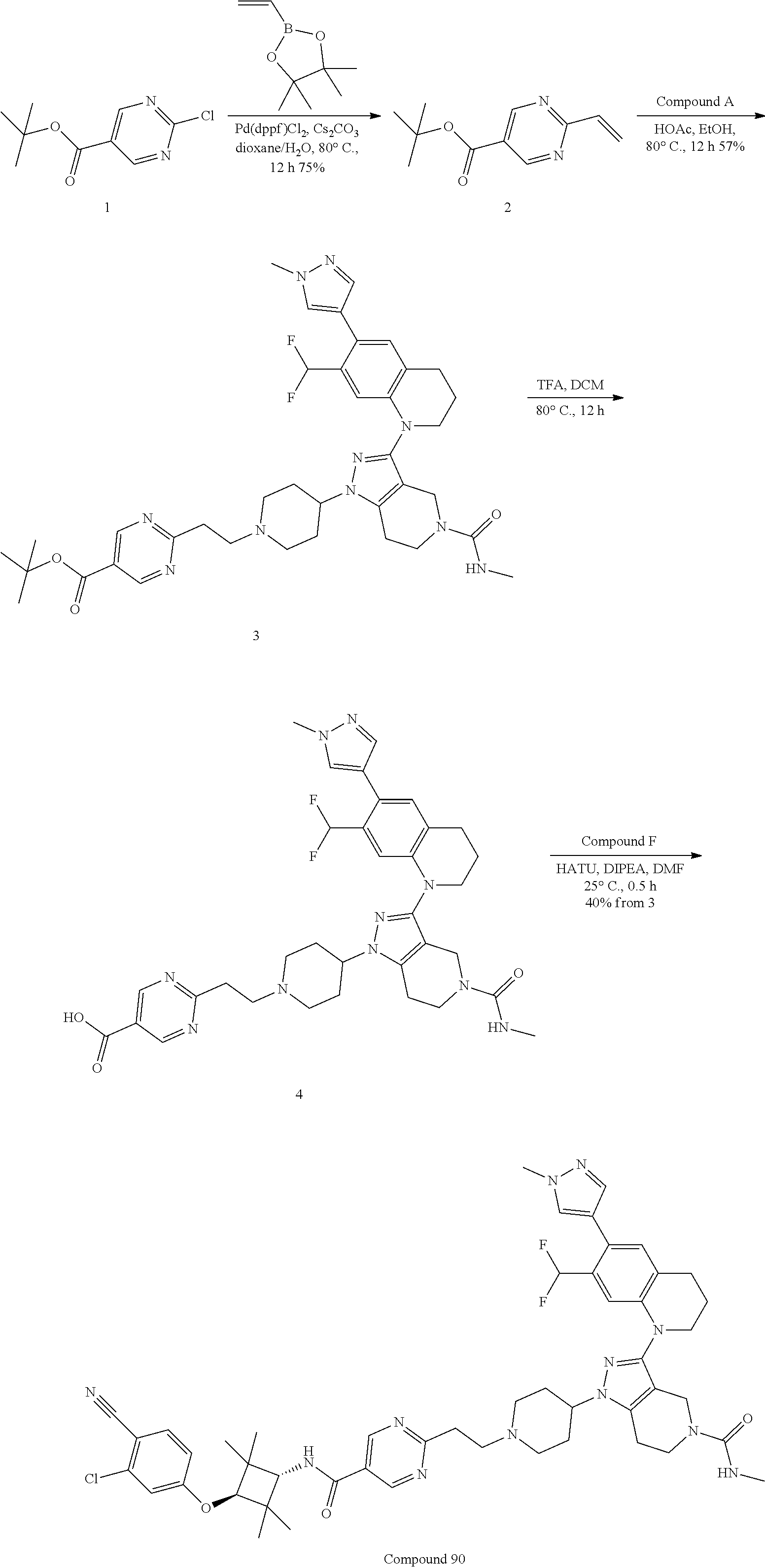 [SMILES: CC(C)(C)OC(=O)c1cnc(Cl)nc1 ; C=CB1OC(C)(C)C(C)(C)O1 ; C=Cc1ncc(C(=O)OC(C)(C)C)cn1 ; CNC(=O)N1CCc2c(c(N3CCCc4cc(-c5cnn(C)c5)c(C(F)F)cc43)nn2C2CCN(CCc3ncc(C(=O)OC(C)(C)C)cn3)CC2)C1 ; CNC(=O)N1CCc2c(c(N3CCCc4cc(-c5cnn(C)c5)c(C(F)F)cc43)nn2C2CCN(CCc3ncc(C(=O)O)cn3)CC2)C1 ; CNC(=O)N1CCc2c(c(N3CCCc4cc(-c5cnn(C)c5)c(C(F)F)cc43)nn2C2CCN(CCc3ncc(C(=O)N[C@H]4C(C)(C)[C@H](Oc5ccc(C#N)c(Cl)c5)C4(C)C)cn3)CC2)C1]

1 was prepared according to the procedure described in US2021/87171 A1. To a mixture of tert-butyl 2-chloropyrimidine-5-carboxylate (5.00 g, 23.3 mmol, 1.0 eq), 4,4,5,5-tetramethyl-2-vinyl-1,3,2-dioxaborolane (5.67 g, 36.9 mmol, 6.25 mL, 1.6 eq), Pd(dppf)Cl₂ (1.75 g, 2.39 mmol, 0.1 eq) in dioxane (75 mL) was degassed and purged with N₂ for 3 times, and then a solution of Cs₂CO₃ (15.2 g, 46.6 mmol, 2.0 eq) in H₂O (15 mL) was added and purged with N₂ for 3 times. The mixture was stirred at 80° C. for 12 h under N₂ atmosphere. The reaction was evaporated under reduced pressure. The residue was purified by column chromatography (silica, petroleum ether/ethyl acetate=10/1 to 3/1). Compound tert-butyl 2-vinylpyrimidine-5-carboxylate (3.60 g, 17.5 mmol, 75% yield) was obtained as a yellow solid. ¹H NMR (400 MHz, CDCl₃): δ=9.17 (s, 2H), 6.99-6.89 (m, 1H), 6.79-6.71 (m, 1H), 5.91-5.82 (m, 1H), 1.62 (s, 9H)

To a mixture of tert-butyl 2-vinylpyrimidine-5-carboxylate (100 mg, 485 μmol, 3.2 eq) in EtOH (3 mL) was added 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (100 mg, 153 μmol, 1.0 eq) and HOAc (31.5 mg, 524 μmol, 30 μL, 3.4 eq). The mixture was heated to 80° C. under N₂ atmosphere for 12 h. The reaction mixture was concentrated under reduced pressure to give a residue. The residue was purified by prep-TLC (silica, DCM/MeOH=10:1). Compound tert-butyl 2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]ethyl]pyrimidine-5-carboxylate (63.0 mg, 86.2 μmol, 57% yield) was obtained as a white solid. LC-MS: MS (ESI⁺): t_{R}=0.536 min, m/z=731.5 [M+H⁺]

To a mixture of tert-butyl 2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]ethyl]pyrimidine-5-carboxylate (63.0 mg, 86.2 μmol, 1.0 eq) in DCM (1 mL) was added TFA (1.54 g, 13.5 mmol, 1 mL, 156 eq). The mixture was heated to 25° C. under nitrogen for 0.5 h. The reaction mixture was concentrated under reduced pressure to give a residue. The crude product 2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]ethyl]pyrimidine-5-carboxylic acid (58.0 mg) was directly used in the next step without further purification. LC-MS: MS (ESI⁺): t_{R}=0.495 min, m/z=675.5 [M+H⁺]

To a solution of 2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]ethyl]pyrimidine-5-carboxylic acid (58.0 mg, 85.96 mol, 1.0 eq) and 4-(3-amino-2,2,4,4-tetramethyl-cyclobutoxy)-2-chloro-benzonitrile (25.0 mg, 89.68 μmol, 1.1 eq) in DMF (0.5 mL) was added HATU (40.0 mg, 105.2 μmol, 1.2 eq) and DIPEA (37.1 mg, 287.1 μmol, 50 μL, 3.3 eq) was added and the mixture was stirred at 25° C. for 0.5 h. The reaction mixture was quenched by addition of H₂O (10 mL) and extracted with DCM (20 mL×3). The organic phase was washed with brine (20 mL), dried over anhydrous Na₂SO₄ and concentrated under reduced pressure to give a residue. The residue was purified by prep-HPLC (FA condition; column: Phenomenex luna C18 150*25 mm*10 um; mobile phase: [water (FA)-ACN]; gradient: 25%-55% B over 10 min). Compound 1-[1-[2-[5-[[3-(3-chloro-4-cyano-phenoxy)-2,2,4,4-tetramethyl-cyclobutyl]carbamoyl]pyrimidin-2-yl]ethyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 90, 32.28 mg, 34.22 μmol, 40% yield over two steps) was obtained as a yellow solid. LC-MS: MS (ESI⁺): t_{R}=1.877 min, m/z=935.6 [M+H⁺]. ¹H NMR (400 MHz, DMSO-d₆): δ=9.04 (s, 2H), 8.22 (t, J=4.4 Hz, 1H), 7.90 (d, J=8.8 Hz, 1H), 7.74 (s, 1H), 7.49 (s, 1H), 7.22 (d, J=2.4 Hz, 1H), 7.08 (s, 1H), 7.04-6.98 (m, 1H), 6.93-6.59 (m, 2H), 6.52 (br d, J=4.4 Hz, 1H), 4.30 (s, 1H), 4.06 (d, J=8.8 Hz, 1H), 4.04-3.96 (m, 3H), 3.86 (s, 3H), 3.61-3.54 (m, 4H), 3.12 (br t, J=7.2 Hz, 2H), 3.02 (br d, J=10.8 Hz, 2H), 2.90-2.80 (m, 4H), 2.71 (br s, 2H), 2.53 (d, J=4.4 Hz, 3H), 2.13 (br t, J=11.2 Hz, 2H), 2.00-1.87 (m, 4H), 1.85-1.77 (m, 2H), 1.23 (s, 6H), 1.12 (s, 6H).

### Example 16: Synthesis of 1-[1-[[5-[[3-(3-chloro-4-cyano-phenoxy)-2,2,4,4-tetramethyl-cyclobutyl]carbamoyl]pyrimidin-2-yl]methyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 91)

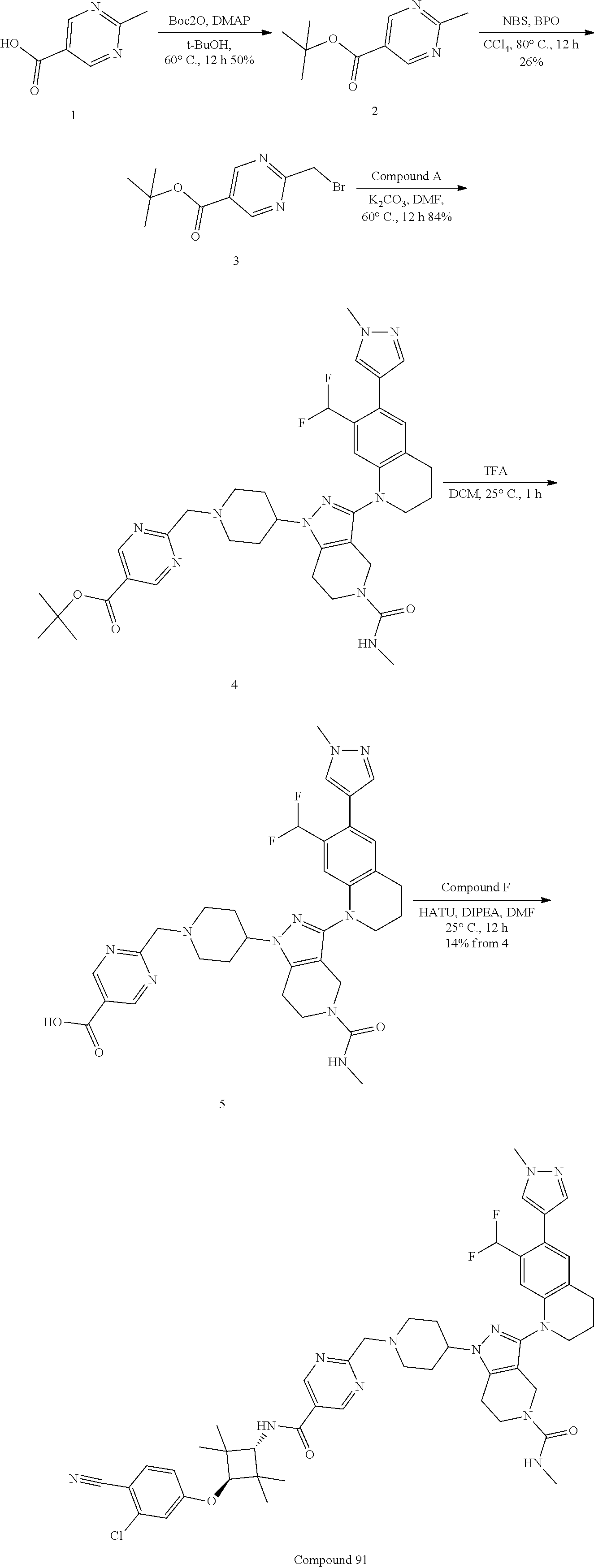

To a solution of 2-methylpyrimidine-5-carboxylic acid (3.00 g, 21.7 mmol, 1.0 eq) in t-BuOH (80 mL) was added Boc₂O (5.69 g, 26.1 mmol, 5.99 mL, 1.2 eq) and DMAP (7.96 g, 65.2 mmol, 3.0 eq). The mixture was stirred at 60° C. for 12 h. The reaction mixture was concentrated to afford crude product. The residue was purified by column chromatography (silica, petroleum ether/ethyl acetate=20/1 to 5/1). Compound tert-butyl 2-methylpyrimidine-5-carboxylate (2.10 g, 10.8 mmol, 50% yield) was obtained as a yellow solid. LC-MS: MS (ESI⁺): t_{R}=0.558 min, m/z=139.2 [M−55]⁺

To a solution of tert-butyl 2-methylpyrimidine-5-carboxylate (0.50 g, 2.57 mmol, 1.0 eq) in CCl₄ (5 mL) was added NBS (549 mg, 3.09 mmol, 1.2 eq) and BPO (62.4 mg, 257 μmol, 0.1 eq). The mixture was stirred at 80° C. for 12 h. The reaction mixture was quenched by addition of Na₂SO₃ (30 mL) at 0° C., and then extracted with CHCl₃ (50 mL×3). The combined organics were washed with H₂O (50 mL×2), dried over anhydrous Na₂SO₄, filtered, and concentrated under reduced pressure to give a residue. The residue was purified by column chromatography (silica, petroleum ether/ethyl acetate=20/1 to 10/1). Compound tert-butyl 2-(bromomethyl)pyrimidine-5-carboxylate (183 mg, 670 μmol, 26% yield) was obtained as a white solid. LC-MS: MS (ESI⁺): t_{R}=0.827 min, m/z=273.2 [M+H⁺]

To a solution of tert-butyl 2-(bromomethyl)pyrimidine-5-carboxylate (50.0 mg, 183 μmol, 1.0 eq), 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (83.3 mg, 159 μmol, 0.8 eq) in DMF (2 mL) was added K₂CO₃ (75.9 mg, 549 μmol, 3.0 eq). The mixture was stirred at 60° C. for 12 h. The residue was diluted with H₂O (100 mL) and extracted with ethyl acetate (100 mL×3). The residue was purified by column chromatography (silica, petroleum ether/ethyl acetate=5/1). Compound tert-butyl 2-[[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]methyl]pyrimidine-5-carboxylate (110 mg, 153 μmol, 84% yield) was obtained as a yellow solid. LC-MS: MS (ESI⁺): t_{R}=0.528 min, m/z=717.5 [M+H⁺]

To a solution of tert-butyl 2-[[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]methyl]pyrimidine-5-carboxylate (130 mg, 181 μmol, 1.0 eq) in DCM (1 mL) was added TFA (1.54 g, 13.46 mmol, 1 mL, 74 eq). The mixture was stirred at 25° C. for 1 h, and then concentrated to give a residue. Compound 2-[[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]methyl]pyrimidine-5-carboxylic acid (110 mg) was obtained as a white solid was directly used for next step without purification. LC-MS: MS (ESI⁺): t_{R}=0.793 min, m/z=661.3 [M+H+]

To a solution of 2-[[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]methyl]pyrimidine-5-carboxylic acid (90 mg of the above material), 4-(3-amino-2,2,4,4-tetramethyl-cyclobutoxy)-2-chloro-benzonitrile (41.8 mg, 150 μmol, 1.1 eq), DIPEA (52.8 mg, 409 μmol, 71.2 μL, 3.0 eq) in DMF (2 mL) was added HATU (62.2 mg, 163 μmol, 1.2 eq) and stirred at 25° C. for 12 h. The residue was diluted with H₂O 10 mL and extracted with Ethyl acetate (10 mL×3). The combined organic layers were washed with brine (10 mL×3), dried over Na₂SO₄, filtered and concentrated under reduced pressure to give a residue. The crude product was purified by reversed-phase HPLC (column: Phenomenex luna C18 150*25 mm*10 um; mobile phase: [water (FA)-ACN]; gradient 24%-54% B over 10 min). Compound 1-[1-[[5-[[3-(3-chloro-4-cyano-phenoxy)-2,2,4,4-tetramethyl-cyclobutyl]carbamoyl]pyrimidin-2-yl]methyl]-4-piperidyl]-3-[7-(difluoromethyl)-6-(1-methylpyrazol-4-yl)-3,4-dihydro-2H-quinolin-1-yl]-N-methyl-6,7-dihydro-4H-pyrazolo[4,3-c]pyridine-5-carboxamide (Compound 91, 19.0 mg, 20.2 μmol, 14% yield over two steps) was obtained as a yellow solid. LC-MS: MS (ESI⁺): t_{R}=1.876 min, m/z=921.7 [M+H+]. ¹H NMR (400 MHz, DMSO-d₆) δ=9.11 (s, 2H), 8.22 (d, J=8.8 Hz, 1H), 8.13 (s, 1H), 7.89 (d, J=8.8 Hz, 1H), 7.74 (s, 1H), 7.49 (s, 1H), 7.22 (d, J=2.4 Hz, 1H), 7.09 (s, 1H), 7.05-6.98 (m, 1H), 6.94-6.61 (m, 2H), 6.49 (br d, J=4.4 Hz, 1H), 4.31 (s, 1H), 4.08 (d, J=9.2 Hz, 1H), 4.01 (s, 3H), 3.86 (s, 5H), 3.64-3.53 (m, 4H), 3.10-2.95 (m, 2H), 2.84 (brt, J=5.6 Hz, 2H), 2.71 (brt, J=4.8 Hz, 2H), 2.54 (d, J=4.4 Hz, 3H), 2.44-2.27 (m, 2H), 2.10-1.93 (m, 4H), 1.91-1.78 (m, 2H), 1.25 (s, 6H), 1.13 (s, 6H).

The compounds below were prepared in a similar manner as described in Examples 1, 2, 3, 4, 8, 15, and 16.

| | | | LC-MS |
|---|---|---|---|
| Compound | Name | ¹H NMR (CDCl₃) | [M + H⁺] |
| 41 | 1-[1-[2-[4-[4-[[3-(3-chloro-4-cyano- | δ = 7.68 (d, J = 8.8 Hz, 2H), 7.57 (d, | 1031.7 |
| | phenoxy)-2,2,4,4-tetramethyl- | J = 8.8 Hz, 1H), 7.53 (s, 1H), 7.40 (s, | |
| | cyclobutyl]carbamoyl]phenyl]piperazin- | 1H), 7.05 (s, 1H), 6.97 (d, J = 2.4 Hz, | |
| | 1-yl]acetyl]-4-piperidyl]-3-[7- | 1H), 6.93-6.85 (m, 3H), 6.84-6.78 | |
| | (difluoromethyl)-6-(1-methylpyrazol-4- | (m, 1H), 6.68-6.36 (m, 1H), 6.12 (d, | |
| | yl)-3,4-dihydro-2H-quinolin-1-yl]-N- | J = 8.0 Hz, 1H), 4.72 (m, 1H), 4.43-4.35 | |
| | methyl-6,7-dihydro-4H-pyrazolo[4,3- | (m, 1H), 4.29 (m, 1H), 4.20-4.09 | |
| | c]pyridine-5-carboxamide | (m, 2H), 4.05 (s, 1H), 4.00-3.91 | |
| | | (m, 5H), 3.87-3.74 (m, 2H), 3.72-3.62 | |
| | | (m, 2H), 3.41-3.28 (m, 5H), | |
| | | 3.26-3.13 (m, 2H), 2.87 (m, 2H), | |
| | | 2.82-2.65 (m, 10H), 2.35-2.17 (m, | |
| | | 1H), 2.15-1.91 (m, 5H), 1.27 (s, 6H), | |
| | | 1.23 (s, 6H) | |
| 42 | 1-[1-[2-[4-[5-[[3-(3-chloro-4-cyano- | (DMSO-d₆) δ = 8.61 (d, J = 2.0 Hz, | 1032.9 |
| | phenoxy)-2,2,4,4-tetramethyl- | 1H), 8.37 (s, 1H), 7.97-7.87 (m, 2H), | |
| | cyclobutyl] carbamoyl]-2-pyridyl] | 7.71 (s, 1H), 7.61 (d, J = 8.8 Hz, 1H), | |
| | piperazin-1-yl] acetyl]-4-piperidyl]-3- | 7.47 (s, 1H), 7.21 (d, J = 2.4 Hz, 1H), | |
| | [7-(difluoromethyl)-6-(1- | 7.08 (s, 1H), 7.03-6.98 (m, 1H), | |
| | methylpyrazol-4-yl)-3,4-dihydro-2H- | 6.90-6.58 (m, 3H), 6.54 (br d, J = 4.8 Hz, | |
| | quinolin-1-yl]-N-methyl-6,7-dihydro- | 1H), 4.49-4.40 (m, 1H), 4.38-4.33 | |
| | 4H-pyrazolo[4,3-c] pyridine-5- | (m, 1H), 4.32-4.29 (m, 1H), 4.20-4.13 | |
| | carboxamide | (m, 1H), 4.05 (br d, J = 9.2 Hz, | |
| | | 1H), 4.01 (s, 2H), 3.85 (s, 3H), 3.71-3.52 | |
| | | (m, 10H), 3.24-3.06 (m, 3H), | |
| | | 2.92-2.68 (m, 6H), 2.21-1.79 (m, | |
| | | 6H), 1.75 (br d, J = 2.4 Hz, 1H), | |
| | | 1.30-1.15 (m, 8H), 1.12 (s, 6H). | |
| 86 | 1-[1-[2-[[5-[[3-(3-chloro-4-cyano- | (DMSO-d6) δ = 9.11 (s, 2H), | 965.7 |
| | phenoxy)-2,2,4,4-tetramethyl- | 8.35-8.32 (m, 1H), 8.24 (d, J = 8.8 Hz, | |
| | cyclobutyl]carbamoyl]pyrimidin-2- | 1H), 7.90 (d, J = 8.4 Hz, 1H), 7.74 (s, | |
| | yl]methoxy]ethyl]-4-piperidyl]-3-[7- | 1H), 7.49 (s, 1H), 7.24-7.21 (m, 1H), | |
| | (difluoromethyl)-6-(1-methylpyrazol-4- | 7.09 (s, 1H), 7.04-6.99 (m, 1H), 6.93- 6.49 | |
| | yl)-3,4-dihydro-2H-quinolin-1-yl]-N- | (m, 3H), 4.73 (s, 2H), 4.31 (s, | |
| | methyl-6,7-dihydro-4H-pyrazolo[4,3- | 1H), 4.07 (d, J = 9.2 Hz, 1H), 4.01 (s, | |
| | c]pyridine-5-carboxamide | 3H), 3.86 (s, 3H), 3.69 (t, J = 6.0 Hz, | |
| | | 2H), 3.61-3.55 (m, 4H), 3.03-2.96 | |
| | | (m, 2H), 2.87-2.81 (m, 2H), 2.75-2.68 | |
| | | (m, 2H), 2.57 (br d, J = 6.4 Hz, | |
| | | 2H), 2.53 (d, J = 4.4 Hz, 3H), 2.18- | |
| | | 2.09 (m, 2H), 2.02-1.94 (m, 4H), | |
| | | 1.84-1.77 (m, 2H), 1.23 (s, 6H), 1.12 | |
| | | (s, 6H) | |
| 87 | 1-[1-[2-[5-[[3-(3-chloro-4-cyano- | δ = 8.91 (s, 2H), 7.64-7.49 (m, 2H), | 951.5 |
| | phenoxy)-2,2,4,4-tetramethyl- | 7.41 (s, 1H), 7.04 (s, 1H), 6.97 (d, J = | |
| | cyclobutyl]carbamoyl]pyrimidin-2- | 2.4 Hz, 1H), 6.86 (s, 1H), 6.84-6.78 | |
| | yl]oxyethyl]-4-piperidyl]-3-[7- | (m, 1H), 6.73-6.37 (m, 1H), 6.10 (m, | |
| | (difluoromethyl)-6-(1-methylpyrazol-4- | 1H), 4.63 (m, 2H), 4.45-4.36 (m, | |
| | yl)-3,4-dihydro-2H-quinolin-1-yl]-N- | 1H), 4.16 (d, J = 8.4 Hz, 1H), 4.07 (s, | |
| | methyl-6,7-dihydro-4H-pyrazolo[4,3- | 1H), 3.99-3.93 (m, 6H), 3.82-3.68 | |
| | c]pyridine-5-carboxamide | (m, 3H), 3.82-3.65 (m, 1H), 3.26-3.15 | |
| | | (m, 2H), 2.94 (s, 2H), 2.87 (m, | |
| | | 2H), 2.80-2.73 (m, 5H), 2.45-2.21 | |
| | | (m, 4H), 2.10-1.92 (m, 4H), 1.28 (s, | |
| | | 6H), 1.24 (s, 6H) | |
| 93 | 1-[1-[5-[[3-(3-chloro-4-cyano- | (400 MHz, METHANOL-d₄) δ = | 907.5 |
| | phenoxy)-2,2,4,4-tetramethyl- | 8.80-8.74 (m, 2H), 7.82-7.76 (m, 1H), | |
| | cyclobutyl]carbamoyl]pyrimidin-2-yl]- | 7.75-7.69 (m, 1H), 7.65-7.60 (m, | |
| | 4-piperidyl]-3-[7-(difluoromethyl)-6-(1- | 1H), 7.51-7.47 (m, 1H), 7.15-7.11 | |
| | methylpyrazol-4-yl)-3,4-dihydro-2H- | (m, 1H), 7.09-7.05 (m, 1H), 7.01-6.94 | |
| | quinolin-1-yl]-N-methyl-6,7-dihydro- | (m, 1H), 6.65 (s, 2H), 5.08-4.99 | |
| | 4H-pyrazolo[4,3-c]pyridine-5- | (m, 3H), 4.30-4.24 (m, 1H), 4.17-4.11 | |
| | carboxamide | (m, 1H), 4.10-4.05 (m, 2H), | |
| | | 3.96-3.89 (m, 3H), 3.78-3.71 (m, | |
| | | 2H), 3.67-3.60 (m, 2H), 3.23-3.22 | |
| | | (m, 1H), 3.23-3.14 (m, 3H), 2.93-2.83 | |
| | | (m, 4H), 2.72-2.66 (m, 3H), | |
| | | 2.10-2.01 (m, 6H), 1.32-1.25 (m, | |
| | | 7H), 1.22-1.20 (m, 6H) | |
| 97 | 1-[1-[2-[3-[[1-[5-[[3-(3-chloro-4-cyano- | δ = 8.75-8.65 (m, 2H), 7.62-7.49 | 1120.7 |
| | phenoxy)-2,2,4,4-tetramethyl- | (m, 2H), 7.46-7.35 (m, 1H), 7.07-7.02 | |
| | cyclobutyl]carbamoyl]pyrimidin-2-yl]- | (m, 1H), 6.99-6.94 (m, 1H), | |
| | 4-piperidyl]methoxy]propoxy]acetyl]-4- | 6.88-6.83 (m, 1H), 6.83-6.78 (m, | |
| | piperidyl]-3-[7-(difluoromethyl)-6-(1- | 1H), 6.72-6.38 (m, 1H), 6.03-5.94 | |
| | methylpyrazol-4-yl)-3,4-dihydro-2H- | (m, 1H), 4.91-4.80 (m, 2H), 4.73-4.60 | |
| | quinolin-1-yl]-N-methyl-6,7-dihydro- | (m, 1H), 4.46-4.37 (m, 1H), | |
| | 4H-pyrazolo[4,3-c]pyridine-5- | 4.23-4.08 (m, 5H), 4.07-4.02 (m, | |
| | carboxamide | 1H), 3.99-3.92 (m, 5H), 3.86-3.76 | |
| | | (m, 2H), 3.75-3.66 (m, 2H), 3.64-3.57 | |
| | | (m, 2H), 3.56-3.47 (m, 2H), | |
| | | 3.33-3.26 (m, 2H), 3.24-3.12 (m, | |
| | | 1H), 3.00-2.82 (m, 5H), 2.79 (d, J = | |
| | | 4.8 Hz, 3H), 2.75 (m, 2H), 2.26-1.94 | |
| | | (m, 7H), 1.93-1.81 (m, 5H), 1.25 (s, | |
| | | 6H), 1.22 (s, 6H). | |
| 98 | 1-[1-[2-[2-[[1-[5-[[3-(3-chloro-4-cyano- | [0001] δ = 8.69 (s, 2H), 7.61-7.52 | 1092.4 |
| | phenoxy)-2,2,4,4-tetramethyl- | (m, 2H), 7.42 (s, 1H), 7.04 (s, | |
| | cyclobutyl]carbamoyl]pyrimidin-2-yl]- | 1H), 6.97 (d, J = 2.4 Hz, 1H), 6.89- | |
| | 4-piperidyl]oxy]ethoxy]acetyl]-4- | 6.77 (m, 2H), 6.55 (m, 1H), 6.01 (m, | |
| | piperidyl]-3-[7-(difluoromethyl)-6-(1- | 1H), 4.65 (m, 1H), 4.42 (m, 1H), 4.33-4.22 | |
| | methylpyrazol-4-yl)-3,4-dihydro-2H- | (m, 4H), 4.20-4.09 (m, 3H), | |
| | quinolin-1-yl]-N-methyl-6,7-dihydro- | 4.05 (s, 1H), 3.96 (s, 5H), 3.81 (m, | |
| | 4H-pyrazolo[4,3-c]pyridine-5- | 1H), 3.78-3.68 (m, 7H), 3.68-3.63 | |
| | carboxamide | (m, 1H), 3.62-3.51 (m, 2H), 3.20 (m, | |
| | | 1H), 2.92-2.82 (m, 3H), 2.79 (d, J = | |
| | | 4.4 Hz, 3H), 2.73 (m, 2H), 2.28-2.10 | |
| | | (m, 2H), 2.09-2.03 (m, 2H), 2.03-1.85 | |
| | | (m, 4H), 1.64 (br s, 2H), 1.26 (s, | |
| | | 6H), 1.22 (s, 6H). | |
| 102 | 1-[1-[2-[2-[3-[4-[5-[[3-(3-chloro-4- | δ = 8.71 (s, 2H), 7.57 (d, J = 8.8 Hz, | 1135.6 |
| | cyano-phenoxy)-2,2,4,4-tetramethyl- | 1H), 7.53 (s, 1H), 7.41 (s, 1H), 7.04 | |
| | cyclobutyl]carbamoyl]pyrimidin-2- | (s, 1H), 6.97 (d, J = 2.4 Hz, 1H), 6.86 | |
| | yl]piperazin-1- | (s, 1H), 6.84-6.78 (m, 1H), 6.71-6.38 | |
| | yl]propoxy]ethoxy]acetyl]-4-piperidyl]- | (m, 1H), 6.09 (d, J = 7.6 Hz, | |
| | 3-[7-(difluoromethyl)-6-(1- | 1H), 4.66 (br d, J = 12.4 Hz, 1H), | |
| | methylpyrazol-4-yl)-3,4-dihydro-2H- | 4.43 (br d, J = 4.4 Hz, 1H), 4.24 (s, | |
| | quinolin-1-yl]-N-methyl-6,7-dihydro- | 2H), 4.18-4.09 (m, 3H), 4.06 (s, 1H), | |
| | 4H-pyrazolo[4,3-c]pyridine-5- | 4.05-3.98 (m, 3H), 3.98-3.95 (m, | |
| | carboxamide | 5H), 3.83-3.74 (m, 2H), 3.73-3.68 | |
| | | (m, 4H), 3.67-3.61 (m, 2H), 3.57 (t, | |
| | | J = 6.4 Hz, 2H), 3.19 (br s, 1H), 2.90-2.81 | |
| | | (m, 3H), 2.78 (d, J = 4.4 Hz, | |
| | | 3H), 2.74 (br t, J = 5.6 Hz, 2H), 2.71-2.59 | |
| | | (m, 5H), 2.24-2.12 (m, 2H), | |
| | | 2.11-2.01 (m, 4H), 2.00-1.95(m, | |
| | | 2H), 1.95-1.92 (m, 1H), 1.26 (s, 6H), | |
| | | 1.22 (s, 6H) | |
| 103 | 1-[1-[2-[2-[2-[4-[5-[[3-(3-chloro-4- | δ = 8.70 (s, 2 H) 7.57 (d, J = 8.8 Hz, | 1121.6 |
| | cyano-phenoxy)-2,2,4,4-tetramethyl- | 1 H) 7.53 (s, 1 H) 7.41 (s, 1 H) 7.27 (s, | |
| | cyclobutyl]carbamoyl]pyrimidin-2- | 2 H) 7.04 (s, 1 H) 6.97 (d, J = 2.4 Hz, | |
| | yl]piperazin-1- | 1 H) 6.85 (s, 1 H) 6.81 (m, 1 H) 6.39-6.69 | |
| | yl]ethoxy]ethoxy]acetyl]-4-piperidyl]-3- | (m, 1 H) 6.12 (d, J = 8.0 Hz, 1 H) | |
| | [7-(difluoromethyl)-6-(1- | 4.67 (d, J = 12.8 Hz, 1 H) 4.44 (q, | |
| | methylpyrazol-4-yl)-3,4-dihydro-2H- | J = 4.4 Hz, 1 H) 4.24 (s, 2 H) 4.09-4.19 | |
| | quinolin-1-yl]-N-methyl-6,7-dihydro- | (m, 3 H) 4.05 (s, 1 H) 3.91-4.00 | |
| | 4H-pyrazolo[4,3-c]pyridine-5- | (m, 8 H) 3.78-3.86 (m, 1 H) 3.66-3.76 | |
| | carboxamide | (m, 9 H) 3.19 (m, 1 H) 2.82-2.90 | |
| | | (m, 3 H) 2.63-2.79 (m, 10 H) | |
| | | 2.11-2.22 (m, 2 H) 2.04-2.08 (m, | |
| | | 2 H) 1.99 (m, 3 H) 1.24 (d, J = 16.4 Hz, | |
| | | 12 H) | |
| 104 | 1-[1-[2-[2-[[1-[5-[[3-(3-chloro-4-cyano- | δ = 8.69 (s, 2H), 7.57 (d, J = 8.8 Hz, | 1106.6 |
| | phenoxy)-2,2,4,4-tetramethyl- | 1H), 7.54 (s, 1H), 7.41 (s, 1H), 7.04 | |
| | cyclobutyl]carbamoyl]pyrimidin-2-yl]- | (s, 1H), 6.97 (d, J = 2.4 Hz, 1H), 6.86 | |
| | 4-piperidyl]methoxy]ethoxy]acetyl]-4- | (s, 1H), 6.83-6.78 (m, 1H), 6.69-6.38 | |
| | piperidyl]-3-[7-(difluoromethyl)-6-(1- | (m, 1H), 6.05 (br d, J = 7.8 Hz, | |
| | methylpyrazol-4-yl)-3,4-dihydro-2H- | 1H), 4.89-4.79 (m, 2H), 4.72-4.62 | |
| | quinolin-1-yl]-N-methyl-6,7-dihydro- | (m, 1H), 4.48-4.39 (m, 1H), 4.24 (s, | |
| | 4H-pyrazolo[4,3-c]pyridine-5- | 2H), 4.19-4.09 (m, 3H), 4.05 (s, 1H), | |
| | carboxamide | 4.01-3.92 (m, 5H), 3.90-3.80 (m, | |
| | | 1H), 3.77-3.67 (m, 5H), 3.66-3.61 | |
| | | (m, 2H), 3.35 (d, J = 6.5 Hz, 2H), | |
| | | 3.26-3.13 (m, 1H), 2.98-2.82 (m, | |
| | | 5H), 2.81-2.72 (m, 6H), 2.25-2.13 | |
| | | (m, 2H), 2.11-2.04 (m, 2H), 2.03-1.89 | |
| | | (m, 4H), 1.88-1.80 (m, 2H), | |
| | | 1.25 (s, 6H), 1.22 (s, 6H) | |
| 105 | 1-[1-[2-[3-[4-[5-[[3-(3-chloro-4-cyano- | δ = 8.73-8.68 (m, 2H), 7.61-7.51 | 1091.6 |
| | phenoxy)-2,2,4,4-tetramethyl- | (m, 2H), 7.42 (s, 1H), 7.06-7.02 (m, | |
| | cyclobutyl]carbamoyl]pyrimidin-2- | 1H), 7.00 (s, 1H), 6.89-6.84 (m, 1H), | |
| | yl]piperazin-1-yl]propoxy]acetyl]-4- | 6.83-6.79 (m, 1H), 6.71-6.38 (m, | |
| | piperidyl]-3-[7-(difluoromethyl)-6-(1- | 1H), 6.10-5.97 (m, 1H), 4.73-4.60 | |
| | methylpyrazol-4-yl)-3,4-dihydro-2H- | (m, 1H), 4.46-4.39 (m, 1H), 4.21 (s, | |
| | quinolin-1-yl]-N-methyl-6,7-dihydro- | 2H), 4.19-4.09 (m, 3H), 4.09-3.99 | |
| | 4H-pyrazolo[4,3-c]pyridine-5- | (m, 4H), 3.98 (s, 2H), 3.96 (s, 3H), | |
| | carboxamide | 3.85-3.75 (m, 2H), 3.74-3.68 (m, | |
| | | 2H), 3.65 (t, J = 6.0 Hz, 2H), 3.27-3.13 | |
| | | (m, 1H), 2.94-2.83 (m, 4H), | |
| | | 2.83-2.77 (m, 5H), 2.75 (t, J = 5.6 | |
| | | Hz, 4H), 2.27-2.12 (m, 2H), 2.11-2.03 | |
| | | (m, 3H), 2.00 (d, J = 8.4 Hz, | |
| | | 4H), 1.26 (s, 6H), 1.23 (s, 6H) | |
| 106 | 1-[1-[2-[2-[4-[5-[[3-(3-chloro-4-cyano- | δ = 8.69 (s, 2 H) 7.57 (d, J = 8.8 Hz, | 1077.7 |
| | phenoxy)-2,2,4,4-tetramethyl- | 1 H) 7.53 (s, 1 H) 7.41 (s, 1 H) 7.03 (s, | |
| | cyclobutyl]carbamoyl]pyrimidin-2- | 1 H) 6.97 (d, J = 2.4 Hz, 1 H) 6.85 (s, | |
| | yl]piperazin-1-yl]ethoxy]acetyl]-4- | 1 H) 6.81 (m, 1 H) 6.34-6.71 (m, 1 H) | |
| | piperidyl]-3-[7-(difluoromethyl)-6-(1- | 5.97 (d, J = 8.00 Hz, 1 H) 4.68 (d, | |
| | methylpyrazol-4-yl)-3,4-dihydro-2H- | J = 12.4 Hz, 1 H) 4.42 (q, J = 4.4 Hz, | |
| | quinolin-1-yl]-N-methyl-6,7-dihydro- | 1 H) 4.23 (d, J = 1.6 Hz, 2 H) 4.06-4.19 | |
| | 4H-pyrazolo[4,3-c]pyridine-5- | (m, 3 H) 4.05 (s, 1 H) 3.90-4.00 (m, | |
| | carboxamide | 9 H) 3.75-3.86 (m, 2 H) 3.67-3.74 | |
| | | (m, 4 H) 3.12-3.25 (m, 1 H) 2.81-2.92 | |
| | | (m, 3 H) 2.78 (d, J = 4.4 Hz, 3 H) | |
| | | 2.73 (m, 4 H) 2.61 (m, 4 H) 2.10-2.27 | |
| | | (m, 2 H) 1.93-2.07 (m, 4 H) | |
| | | 1.25 (s, 6 H) 1.22 (s, 6 H). | |
| 107 | 1-[1-[2-[[1-[5-[[3-(3-chloro-4-cyano- | δ = 8.69 (s, 2H), 7.61-7.51 (m, 2H), | 1062.4 |
| | phenoxy)-2,2,4,4-tetramethyl- | 7.41 (s, 1H), 7.04 (s, 1H), 6.97 (d, J = | |
| | cyclobutyl] carbamoyl] pyrimidin-2-yl]- | 2.4 Hz, 1H), 6.86 (s, 1H), 6.84-6.77 | |
| | 4-piperidyl] methoxy] acetyl]-4- | (m, 1H), 6.72-6.39 (m, 1H), 5.97 (d, | |
| | piperidyl]-3-[7-(difluoromethyl)-6-(1- | J = 8.2 Hz, 1H), 4.86 (br d, J = 12.8 | |
| | methylpyrazol-4-yl)-3,4-dihydro-2H- | Hz, 2H), 4.67 (br d, J = 11.8 Hz, 1H), | |
| | quinolin-1-yl]-N-methyl-6,7-dihydro- | 4.49-4.38 (m, 1H), 4.18 (s, 2H), 4.13 | |
| | 4H-pyrazolo[4,3-c] pyridine-5- | (br d, J = 8.0 Hz, 2H), 4.05 (s, 1H), | |
| | carboxamide | 3.97 (s, 2H), 3.96-3.93 (m, 3H), 3.87-3.75 | |
| | | (m, 2H), 3.74-3.64 (m, 2H), | |
| | | 3.47-3.35 (m, 2H), 3.19 (br t, J = | |
| | | 12.4 Hz, 1H), 2.95 (br t, J = 11.6 Hz, | |
| | | 2H), 2.91-2.81 (m, 3H), 2.79 (d, J = | |
| | | 4.6 Hz, 3H), 2.75 (br t, J = 5.4 Hz, | |
| | | 2H), 2.27-2.09 (m, 2H), 2.09-2.04 | |
| | | (m, 2H), 2.04-1.95 (m, 3H), 1.93-1.84 | |
| | | (m, 2H), 1.66 (brs, 3H), 1.25 (s, | |
| | | 6H), 1.22 (s, 6H) | |
| 108 | 1-[1-[3-[4-[5-[[3-(3-chloro-4-cyano- | δ = 8.71 (s, 2 H), 7.64-7.50 (m, 2 H), | 1047.4 |
| | phenoxy)-2,2,4,4-tetramethyl- | 7.41 (s, 1 H), 7.10-6.93 (m, 2 H), | |
| | cyclobutyl]carbamoyl]pyrimidin-2- | 6.92-6.75 (m, 2 H), 6.74-6.35 (m, | |
| | yl]piperazin-1-yl]propanoyl]-4- | 1 H), 6.04-5.90 (m, 1 H), 4.82-4.63 (m, | |
| | piperidyl]-3-[7-(difluoromethyl)-6-(1- | 1 H), 4.50-4.33 (m, 1 H), 4.30-3.88 | |
| | methylpyrazol-4-yl)-3,4-dihydro-2H- | (m, 13 H), 3.85-3.65 (m, 4 H), 3.30-3.13 | |
| | quinolin-1-yl]-N-methyl-6,7-dihydro- | (m, 1 H), 2.98-2.53 (m, 16 H), | |
| | 4H-pyrazolo[4,3-c]pyridine-5- | 2.22-1.96 (m, 5 H), 1.27-1.17 (m, | |
| | carboxamide | 12 H). | |

### Example 17: Synthesis of 2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carbonyl]-1-piperidyl]-N-[3-(3-chloro-4-cyano-phenoxy)-2,2,4,4-tetramethyl-cyclobutyl]pyrimidine-5-carboxamide (Compound 22)

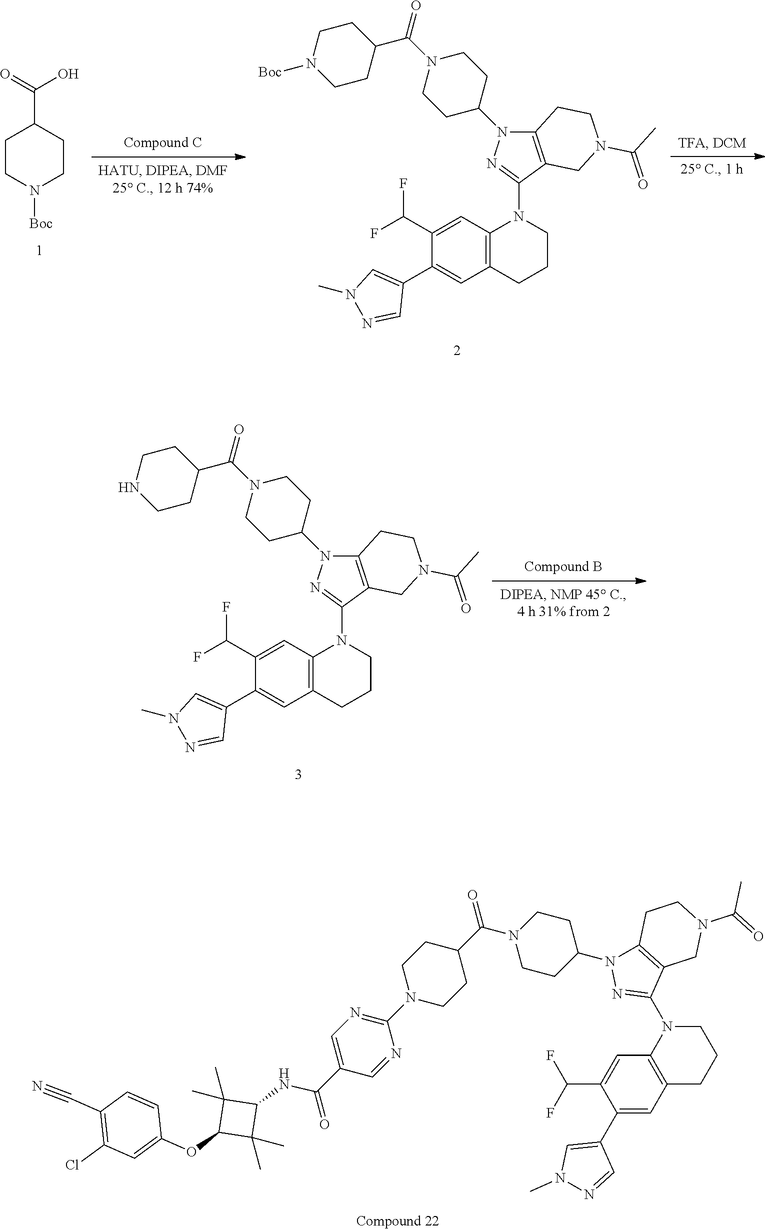 [SMILES: CC(C)(C)OC(=O)N1CCC(C(=O)O)CC1 ; CC(=O)N1CCc2c(c(N3CCCc4cc(-c5cnn(C)c5)c(C(F)F)cc43)nn2C2CCN(C(=O)C3CCN(C(=O)OC(C)(C)C)CC3)CC2)C1 ; CC(=O)N1CCc2c(c(N3CCCc4cc(-c5cnn(C)c5)c(C(F)F)cc43)nn2C2CCN(C(=O)C3CCNCC3)CC2)C1 ; CC(=O)N1CCc2c(c(N3CCCc4cc(-c5cnn(C)c5)c(C(F)F)cc43)nn2C2CCN(C(=O)C3CCN(c4ncc(C(=O)N[C@H]5C(C)(C)[C@H](Oc6ccc(C#N)c(Cl)c6)C5(C)C)cn4)CC3)CC2)C1]

To a solution of 1-tert-butoxycarbonylpiperidine-4-carboxylic acid (23 mg, 100.08 μmol, 1 eq) DIPEA (39 mg, 300.24 μmol, 0.05 mL, 3 eq) in DMF (0.5 mL) was added 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (60 mg, 100.08 μmol, 1 eq) and HATU (42 mg, 110.09 μmol, 1.1 eq). The mixture was stirred at 25° C. for 12 h. The reaction was diluted with water (100 mL) and the resulting mixture was extracted with ethyl acetate (100 mL×2). The combined organic layers were dried over Na₂SO₄ and concentrated to give the residue. The residue was purified by prep-TLC (silica, dichloromethane/methyl alcohol=10:1). Compound tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carbonyl]piperidine-1-carboxylate (56 mg, 74.56 μmol, 74% yield) was obtained as a white solid.

LC-MS: MS (ES⁺): t_{R}=0.586 min, m/z=721.6 [M+H⁺]

To a solution of tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carbonyl]piperidine-1-carboxylate (56 mg, 74.56 μmol, 1 eq) in DCM (1 mL) was added TFA (737 mg, 6.46 mmol, 0.5 mL, 86.64 eq). The mixture was stirred at 25° C. for 1 h. The mixture was concentrated to give a residue. Compound 1-[3-[7-(difluoromethyl)-6-(1-methylpyrazol-4-yl)-3,4-dihydro-2H-quinolin-1-yl]-1-[1-(piperidine-4-carbonyl)-4-piperidyl]-6,7-dihydro-4H-pyrazolo[4,3-c]pyridin-5-yl]ethanone (54 mg) was obtained as a white solid and directly used in the next step without further purification.

LC-MS: MS (ES⁺): t_{R}=0.489 min, m/z=621.6 [M+H⁺]

To a solution of 1-[3-[7-(difluoromethyl)-6-(1-methylpyrazol-4-yl)-3,4-dihydro-2H-quinolin-1-yl]-1-[1-(piperidine-4-carbonyl)-4-piperidyl]-6,7-dihydro-4H-pyrazolo[4,3-c]pyridin-5-yl]ethanone (54 mg) in NMP (0.5 mL) was added DIPEA (28.50 mg, 220.48 μmol, 0.04 mL, 3 eq) and 2-chloro-N-[3-(3-chloro-4-cyano-phenoxy)-2,2,4,4-tetramethyl-cyclobutyl]pyrimidine-5-carboxamide (31 mg, 73.49 μmol, 1 eq). The mixture was stirred at 45° C. for 4 h. The reaction mixture was concentrated to give the residue. The residue was purified by prep-HPLC (column: Phenomenex Luna C18 150*25 mm*10 um; mobile phase: [water(FA)-ACN]; gradient: 53%-83% B over 10 min) to give 2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carbonyl]-1-piperidyl]-N-[3-(3-chloro-4-cyano-phenoxy)-2,2,4,4-tetramethyl-cyclobutyl]pyrimidine-5-carboxamide (23 mg, 22.85 μmol, 31% yield over two steps) was obtained as an off-white solid

¹H NMR (400 MHz, CDCl₃): δ=8.63 (s, 2H), 7.50 (d, J=8.8 Hz, 1H), 7.48-7.45 (m, 1H), 7.35-7.32 (m, 1H), 6.96 (d, J=21.6 Hz, 1H), 6.89 (d, J=2.4 Hz, 1H), 6.83-6.79 (m, 1H), 6.73 (d, J=2.4, 8.8 Hz, 1H), 6.63-6.30 (m, 1H), 5.86 (d, J=8.4 Hz, 1H), 4.81 (d, J=13.2 Hz, 2H), 4.72-4.62 (m, 1H), 4.28-4.12 (m, 1H), 4.11-4.01 (m, 4H), 3.97 (s, 1H), 3.88 (d, J=1.8 Hz, 3H), 3.87-3.74 (m, 1H), 3.73-3.66 (m, 1H), 3.66-3.57 (m, 2H), 3.27-3.11 (m, 1H), 3.07-2.94 (m, 2H), 2.84-2.66 (m, 6H), 2.25-2.12 (m, 1H), 2.10 (s, 1H), 2.06-1.95 (m, 6H), 1.94-1.87 (m, 1H), 1.76 (s, 4H), 1.18 (s, 6H), 1.15 (s, 6H)

LC-MS: MS (ES⁺): t_{R}=2.781 min, m/z=1003.4 [M+H⁺]

### Example 18: Synthesis of 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]-N-[3-(4-cyano-3-methoxy-phenoxy)-2,2,4,4-tetramethyl-cyclobutyl]benzamide (Compound 185)

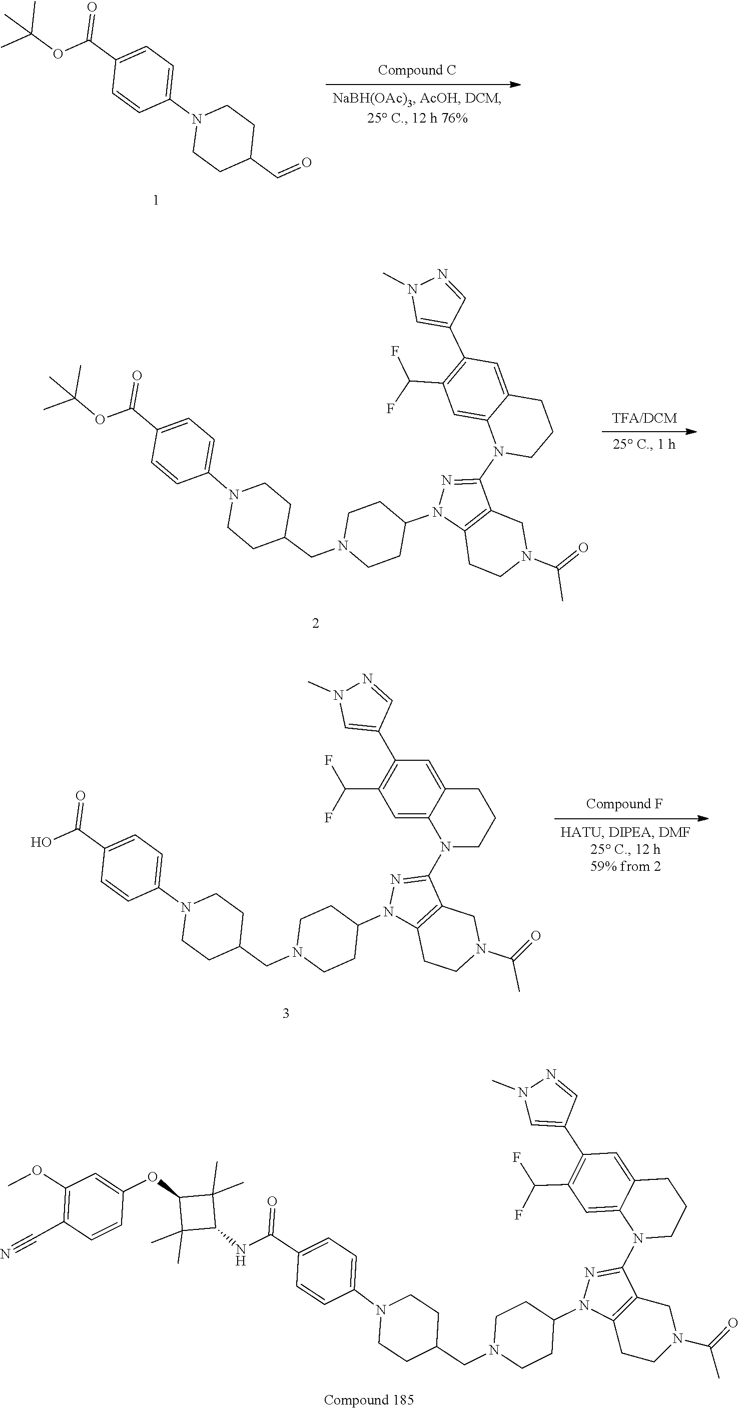 [SMILES: CC(C)(C)OC(=O)c1ccc(N2CCC(C=O)CC2)cc1 ; CC(=O)N1CCc2c(c(N3CCCc4cc(-c5cnn(C)c5)c(C(F)F)cc43)nn2C2CCN(CC3CCN(c4ccc(C(=O)OC(C)(C)C)cc4)CC3)CC2)C1 ; CC(=O)N1CCc2c(c(N3CCCc4cc(-c5cnn(C)c5)c(C(F)F)cc43)nn2C2CCN(CC3CCN(c4ccc(C(=O)O)cc4)CC3)CC2)C1 ; COc1cc(O[C@H]2C(C)(C)[C@H](NC(=O)c3ccc(N4CCC(CN5CCC(n6nc(N7CCCc8cc(-c9cnn(C)c9)c(C(F)F)cc87)c7c6CCN(C(C)=O)C7)CC5)CC4)cc3)C2(C)C)ccc1C#N]

Synthesis of 1 was reported in US2018/215731 A1.

To a solution of tert-butyl 4-(4-formyl-1-piperidyl)benzoate (80 mg, 276 μmol, 1.4 eq) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (100 mg, 196 μmol, 1.0 eq) in DCM (8 mL) was added AcOH (20 mg, 333 μmol, 1.7 eq) and stirred at 25° C. for 0.5 h, NaBH(OAc)₃ (200 mg, 944 μmol, 4.8 eq) was added to the mixture and stirred at 25° C. for 12 h. The mixture was slowly poured into NaHCO₃ solution (80 mL). The aqueous phase was extracted with DCM (40 mL×3). The combined organic phase was washed with brine (30 mL×2), dried with anhydrous sodium sulfate, filtered, and concentrated under vacuum. The residue was purified by column chromatography (SiO₂, Petroleum ether: Ethyl acetate=1:0 to 3:1 and Dichloromethane:Methanol=10:1). Compound tert-butyl 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]benzoate (117 mg, 149 μmol, 76% yield) was obtained as a colorless oil.

LC-MS: MS (ESI⁺): t_{R}=0.559 min, m/z=783.5 [M+H⁺]

To a solution of tert-butyl 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]benzoate (100 mg, 128 μmol, 1.0 eq) in DCM (5 mL) was added TFA (4.60 g, 40.4 mmol, 316.2 eq) and stirred at 25° C. for 1 h. The reaction mixture was concentrated under reduced pressure to give a residue. Compound 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]benzoic acid (103 mg) was obtained as a yellow solid and directly used in the next step without further purification.

LC-MS: MS (ESI⁺): t_{R}=0.470 min, m/z=727.5 [M+H⁺]

To a solution of 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]benzoic acid (60 mg, 71.4 μmol, 1.0 eq), 4-(3-amino-2,2,4,4-tetramethyl-cyclobutoxy)-2-methoxy-benzonitrile (40 mg, 103 μmol, 1.4 eq) in DMF (3 mL) was added DIPEA (74 mg, 574 μmol, 8.1 eq) and HATU (41 mg, 107 μmol, 1.5 eq) and stirred at 25° C. for 1 h. The mixture was diluted with ethyl acetate (100 mL), washed with water (15 mL×2), brine (15 mL), dried with anhydrous sodium sulfate, filtered, and concentrated under vacuum. The crude product was purified by reversed-phase HPLC (column: Phenomenex luna C18 150×25 mm×10 um; mobile phase: [water (FA)-ACN]; gradient: 30%-60% B over 10 min). Compound 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]-N-[3-(4-cyano-3-methoxy-phenoxy)-2,2,4,4-tetramethyl-cyclobutyl]benzamide (41.75 mg, 41.8 μmol, 59% yield over two steps) was obtained as an off-white solid.

¹H NMR (400 MHz, CHLOROFORM-d) δ ppm 7.68 (d, J=8.8 Hz, 2H) 7.54 (d, J=6.0 Hz, 1H) 7.46 (d, J=8.8 Hz, 1H) 7.41 (d, J=6.8 Hz, 1H) 7.04 (d, J=19.2 Hz, 1H) 6.91 (m, 3H) 6.36-6.72 (m, 3H) 6.12 (m, 1H) 4.30 (s, 1H) 4.12-4.19 (m, 2H) 4.05 (s, 1H) 3.96 (s, 3H) 3.88-3.94 (m, 4H) 3.84 (m, 2H) 3.64-3.79 (m, 3H) 3.15 (s, 2H) 2.73-2.92 (m, 6H) 2.34-2.56 (m, 3H) 2.23-2.32 (m, 2H) 2.18 (s, 2H) 2.02-2.13 (m, 5H) 1.87-1.99 (m, 4H) 1.31-1.39 (m, 2H) 1.25 (d, J=10.4 Hz, 12H).

LC-MS: MS (ESI⁺): t_{R}=1.992 min, m/z=983.8 [M+H⁺]

The compounds below were prepared in a similar manner as described in Examples 5, 6, 7, 9, 11, 17, and 18.

| | | | LC-MS |
|---|---|---|---|
| Compound | Name | ¹H NMR (CDCl₃) | [M + H⁺] |
| 10 | 2-[4-[[3-[5-acetyl-3-[7- | δ = 8.69 (s, 2H), 7.57 (d, J = 8.8 Hz, | 961.5 |
| | (difluoromethyl)-6-(1-methylpyrazol- | 1H), 7.54 (d, J = 5.6 Hz, 1H), 7.42 (d, | |
| | 4-yl)-3,4-dihydro-2H-quinolin-1-yl]- | J = 5.6 Hz, 1H), 7.12 (d, J = 16.0 Hz, | |
| | 6,7-dihydro-4H-pyrazolo[4,3- | 1H), 7.04 (d, J = 8.8 Hz, 1H), 6.97 (d, | |
| | c]pyridin-1-yl]azetidin-1-yl]methyl]- | J = 2.4 Hz, 1H), 6.81 (dd, J = 2.4, 8.8 | |
| | 1-piperidyl]-N-[3-(3-chloro-4-cyano- | Hz, 1H), 6.71-6.39 (m, 1H), 5.98 (br | |
| | phenoxy)-2,2,4,4-tetramethyl- | d, J = 8.0 Hz, 1H), 5.22-5.04 (m, 1H), | |
| | cyclobutyl]pyrimidine-5-carboxamide | 4.90 (br d, J = 12.8 Hz, 2H), 4.70- | |
| | | 4.43 (m, 2H), 4.29 (s, 1H), 4.21-4.03 | |
| | | (m, 5H), 3.97 (s, 3H), 3.88 (br t, J = 5.6 | |
| | | Hz, 1H), 3.78-3.69 (m, 3H), 3.06 (br | |
| | | d, J = 6.4 Hz, 1H), 3.00-2.85 (m, 5H), | |
| | | 2.79-2.68 (m, 2H), 2.19-1.97 (m, | |
| | | 7H), 1.90 (br d, J = 12.0 Hz, 2H), 1.32- | |
| | | 1.28 (m, 1H), 1.23 (d, J = 15.2 Hz, 12H). | |
| 11 | 2-[4-[3-[5-acetyl-3-[7- | δ = 8.70 (s, 2H), 7.58 (d, J = 8.8 Hz, | 947.5 |
| | (difluoromethyl)-6-(1-methylpyrazol- | 1H), 7.55 (d, J = 4.4 Hz, 1H), 7.42 (d, J = | |
| | 4-yl)-3,4-dihydro-2H-quinolin-1-yl]- | 4.8 Hz, 1H), 7.12 (br d, J = 5.6 Hz, | |
| | 6,7-dihydro-4H-pyrazolo[4,3- | 1H), 7.07 (s, 1H), 6.97 (d, J = 2.4 Hz, | |
| | c]pyridin-1-yl]azetidin-1-yl]-1- | 1H), 6.81 (dd, J = 2.4, 8.8 Hz, 1H), | |
| | piperidyl]-N-[3-(3-chloro-4-cyano- | 6.73-6.42 (m, 1H), 5.99 (d, J = 8.0 | |
| | phenoxy)-2,2,4,4-tetramethyl- | Hz, 1H), 5.36-5.12 (m, 1H), 5.02- | |
| | cyclobutyl]pyrimidine-5-carboxamide | 4.84 (m, 2H), 4.68-4.41 (m, 2H), 4.33 | |
| | | (s, 1H), 4.32-4.25 (m, 1H), 4.17- | |
| | | 4.11 (m, 2H), 4.06 (s, 1H), 3.97 (d, J = | |
| | | 1.6 Hz, 3H), 3.89 (br t, J = 6.0 Hz, 1H), | |
| | | 3.74 (q, J = 6.0 Hz, 3H), 3.35-3.16 | |
| | | (m, 1H), 3.10-2.98 (m, 2H), 2.95- | |
| | | 2.87 (m, 2H), 2.82-2.69 (m, 2H), 2.17 | |
| | | (s, 2H), 2.14-2.08 (m, 2H), 2.07- | |
| | | 1.96 (m, 4H), 1.68-1.60 (m, 2H), 1.25 | |
| | | (d, J = 17.6 Hz, 12H). | |
| 12 | 2-[4-[3-[5-acetyl-3-[7- | δ = 8.70 (s, 2 H) 7.49-7.61 (m, 2 H) | 961.7 |
| | (difluoromethyl)-6-(1-methylpyrazol- | 7.41 (d, J = 6.0 Hz, 1 H) 6.90-7.08 (m, | |
| | 4-yl)-3,4-dihydro-2H-quinolin-1-yl]- | 3 H) 6.81 (m, 1 H) 6.38-6.71 (m, 1 H) | |
| | 6,7-dihydro-4H-pyrazolo[4,3- | 5.94 (d, J = 8.4 Hz, 1 H) 4.69-4.86 (m, | |
| | c]pyridin-1-yl]pyrrolidin-1-yl]-1- | 3 H) 4.09-4.32 (m, 3 H) 4.05 (s, 1 H) | |
| | piperidyl]-N-[3-(3-chloro-4-cyano- | 3.96 (d, J = 1.6 Hz, 3 H) 3.84-3.94 (m, | |
| | phenoxy)-2,2,4,4-tetramethyl- | 1 H) 3.63-3.78 (m, 3 H) 3.32-3.52 | |
| | cyclobutyl]pyrimidine-5-carboxamide | (m, 1 H) 3.01-3.21 (m, 3 H) 2.84- | |
| | | 2.99 (m, 4 H) 2.73-2.83 (m, 2 H) 2.53- | |
| | | 2.71 (m, 1 H) 2.30-2.44 (m, 2 H) | |
| | | 2.17 (s, 1 H) 1.97-2.12 (m, 6 H) 1.54- | |
| | | 1.62 (m, 2 H) 1.26 (s, 6 H) 1.22 (s, 6 H) | |
| 13 | 2-[4-[[4-[5-acetyl-3-[6- | (DMSO-d₆) δ = 8.75 (s, 2 H) 7.90 (d, | 975.7 |
| | (difluoromethyl)-5-(1-methylpyrazol- | J = 8.8 Hz, 1 H) 7.68-7.79 (m, 3 H) | |
| | 4-yl)indolin-1-yl]-6,7-dihydro-4H- | 7.52 (s, 1 H) 7.17-7.23 (m, 2 H) 6.68- | |
| | pyrazolo[4,3-c]pyridin-1-yl]-1- | 7.03 (m, 2 H) 4.74 (m, 2 H) 4.55 (m, | |
| | piperidyl]methyl]-1-piperidyl]-N-[3- | 2 H) 4.29 (s, 1 H) 3.94-4.13 (m, 4 H) | |
| | (3-chloro-4-cyano-phenoxy)-2,2,4,4- | 3.88 (s, 3 H) 3.66-3.79 (m, 2 H) 3.13- | |
| | tetramethyl-cyclobutyl]pyrimidine-5- | 3.23 (m, 3 H) 2.89-3.06 (m, 5 H) | |
| | carboxamide | 2.81 (m, 1 H) 2.67 (m, 1 H) 2.19 (m, 2 | |
| | | H) 2.10 (m, 3 H) 2.03-2.07 (m, 3 H) | |
| | | 1.82 (m, 4 H) 1.21 (s, 6 H) 1.11 (s, 6 | |
| | | H) 0.99-1.08 (m, 2 H) | |
| 15 | 2-[2-[4-[[4-[5-acetyl-3-[7- | (DMSO-d₆) δ = 9.05 (s, 2H), 8.23 (d, J = | 1001.4 |
| | (difluoromethyl)-6-(1-methylpyrazol- | 8.8 Hz, 1H), 8.02-7.93 (m, 1H), | |
| | 4-yl)-3,4-dihydro-2H-quinolin-1-yl]- | 7.74 (s, 1H), 7.54-7.44 (m, 1H), 7.21 | |
| | 6,7-dihydro-4H-pyrazolo[4,3-c] | (d, J = 2.0 Hz, 1H), 7.09 (s, 1H), 7.04- | |
| | pyridin-1-yl]-1-piperidyl]methyl] | 6.97 (m, 1H), 6.95-6.59 (m, 2H), 4.87 | |
| | triazol-1-yl]ethyl]-N-[3-(3-chloro-4- | (br t, J = 6.8 Hz, 2H), 4.35-4.25 (m, | |
| | cyano-phenoxy)-2,2,4,4-tetramethyl- | 1H), 4.16-4.08 (m, 2H), 4.05 (d, J = | |
| | cyclobutyl]pyrimidine-5- | 9.2 Hz, 1H), 4.01-3.91 (m, 1H), 3.86 | |
| | carboxamide | (s, 3H), 3.76-3.65 (m, 2H), 3.56 (br d, | |
| | | J = 10.8 Hz, 6H), 2.91-2.70 (m, 6H), | |
| | | 2.09-1.90 (m, 9H), 1.86-1.74 (m, | |
| | | 2H), 1.21 (s, 6H), 1.09 (s, 6H). | |
| 18 | 2-[5-[4-[5-acetyl-3-[7- | 8 = 9.06 (s, 2H), 7.56 (d, J = 8.4 Hz, | 968.7 |
| | (difluoromethyl)-6-(1-methylpyrazol- | 1H), 7.52 (br s, 1H), 7.39 (s, 1H), 7.08- | |
| | 4-yl)-3,4-dihydro-2H-quinolin-1-yl]- | 7.00 (m, 1H), 6.98 (d, J = 2.4 Hz, | |
| | 6,7-dihydro-4H-pyrazolo[4,3- | 1H), 6.94-6.86 (m, 1H), 6.84-6.79 | |
| | c]pyridin-1-yl]-1-piperidyl]pent-3- | (m, 1H), 6.70-6.34 (m, 2H), 4.29 (br | |
| | ynyl]-N-[3-(3-chloro-4-cyano- | s, 1H), 4.18 (br d, J = 8.5 Hz, 1H), 4.15- | |
| | phenoxy)-2,2,4,4-tetramethyl- | 4.03 (m, 3H), 3.95 (s, 3H), 3.93- | |
| | cyclobutyl]pyrimidine-5-carboxamide | 3.87 (m, 1H), 3.79-3.65 (m, 3H), 3.54- | |
| | | 3.38 (m, 3H), 3.32-3.13 (m, 4H), | |
| | | 2.93-2.72 (m, 7H), 2.36-2.23 (m, | |
| | | 2H), 2.19-2.04 (m, 7H), 1.30 (s, 6H), | |
| | | 1.25 (s, 6H) | |
| 19 | 2-[3-[4-[5-acetyl-3-[7- | δ = 9.08 (s, 2H), 7.63-7.50 (m, 2H), | 930.5 |
| | (difluoromethyl)-6-(1-methylpyrazol- | 7.44-7.37 (m, 1H), 7.08-6.95 (m, | |
| | 4-yl)-3,4-dihydro-2H-quinolin-1-yl]- | 2H), 6.90-6.78 (m, 2H), 6.72-6.33 | |
| | 6,7-dihydro-4H-pyrazolo[4,3- | (m, 1H), 6.24-6.11 (m, 1H), 4.28- | |
| | c]pyridin-1-yl]-1-piperidyl]prop-1- | 4.07 (m, 4H), 4.02-3.86 (m, 5H), 3.81- | |
| | ynyl]-N-[3-(3-chloro-4-cyano- | 3.65 (m, 5H), 3.17-3.01 (m, 2H), | |
| | phenoxy)-2,2,4,4-tetramethyl- | 2.92-2.71 (m, 4H), 2.70-2.53 (m, | |
| | cyclobutyl]pyrimidine-5-carboxamide | 2H), 2.43-2.26 (m, 2H), 2.17 (s, 1H), | |
| | | 2.11-1.92 (m, 6H), 1.30 (s, 6H), 1.25 | |
| | | (s, 6H) | |
| 20 | 2-[3-[[4-[5-acetyl-3-[7- | δ = 8.74-8.66 (m, 2H), 7.61-7.50 (m, | 961.6 |
| | (difluoromethyl)-6-(1-methylpyrazol- | 2H), 7.43-7.37 (m, 1H), 7.08-6.94 | |
| | 4-yl)-3,4-dihydro-2H-quinolin-1-yl]- | (m, 2H), 6.91-6.85 (m, 1H), 6.83- | |
| | 6,7-dihydro-4H-pyrazolo[4,3- | 6.77 (m, 1H), 6.72-6.35 (m, 1H), 5.93 | |
| | c]pyridin-1-yl]-1- | (m, 1H), 4.75 (s, 1H), 4.38-4.28 (m, | |
| | piperidyl]methyl]azetidin-1-yl]-N-[3- | 2H), 4.28-4.20 (m, 1H), 4.16-4.10 | |
| | (3-chloro-4-cyano-phenoxy)-2,2,4,4- | (m, 2H), 4.05 (s, 1H), 4.01-3.94 (m, | |
| | tetramethyl-cyclobutyl]pyrimidine-5- | 4H), 3.93-3.86 (m, 3H), 3.80-3.66 | |
| | carboxamide | (m, 3H), 3.15-2.94 (m, 3H), 2.93- | |
| | | 2.72 (m, 6H), 2.39-2.21 (m, 4H), 2.17 | |
| | | (s, 1H), 2.11-2.02 (m, 4H), 2.00- | |
| | | 1.86 (m, 2H), 1.25 (s, 6H), 1.22 (s, 6H) | |
| 21 | 2-[4-[[3-[5-acetyl-3-[7- | δ = 8.70 (s, 2H), 7.62-7.51 (m, 2H), | 975.6 |
| | (difluoromethyl)-6-(1-methylpyrazol- | 7.41 (d, J = 6.8 Hz, 1H), 7.09-6.96 | |
| | 4-yl)-3,4-dihydro-2H-quinolin-1-yl]- | (m, 2H), 6.94-6.88 (m, 1H), 6.85- | |
| | 6,7-dihydro-4H-pyrazolo[4,3- | 6.78 (m, 1H), 6.71-6.36 (m, 1H), 5.96- | |
| | c]pyridin-1-yl]pyrrolidin-1- | 5.87 (m, 1H), 4.92-4.80 (m, 2H), | |
| | yl]methyl]-1-piperidyl]-N-[3-(3- | 4.79-4.67 (m, 1H), 4.26 (m, 1H), 4.16- | |
| | chloro-4-cyano-phenoxy)-2,2,4,4- | 4.09 (m, 2H), 4.05 (s, 1H), 3.99- | |
| | tetramethyl-cyclobutyl]pyrimidine-5- | 3.91 (m, 4H), 3.85 (m, 1H), 3.79-3.67 | |
| | carboxamide | (m, 3H), 3.21-3.08 (m, 1H), 3.03- | |
| | | 2.66 (m, 9H), 2.53-2.28 (m, 4H), 2.17 | |
| | | (s, 1H), 2.12-2.00 (m, 4H), 1.98- | |
| | | 1.87 (m, 2H), 1.86-1.72 (m, 1H), 1.25 | |
| | | (s, 6H), 1.22 (s, 6H) | |
| 24 | 2-[4-[[4-[5-acetyl-3-[6-(1- | (DMSO-d6) δ = 8.74 (s, 2H), 7.94- | 939.7 |
| | methylpyrazol-4-yl)-3,4-dihydro-2H- | 7.86 (m, 2H), 7.73-7.64 (m, 2H), 7.24- | |
| | quinolin-1-yl]-6,7-dihydro-4H- | 7.16 (m, 2H), 7.14-7.06 (m, 1H), | |
| | pyrazolo[4,3-c]pyridin-1-yl]-1- | 7.03-6.98 (m, 1H), 6.46-6.38 (m, | |
| | piperidyl]methyl]-1-piperidyl]-N-[3- | 1H), 4.73 (br d, J = 12.8 Hz, 2H), 4.28 | |
| | (3-chloro-4-cyano-phenoxy)-2,2,4,4- | (s, 1H), 4.15-4.07 (m, 2H), 4.05- | |
| | tetramethyl-cyclobutyl]pyrimidine-5- | 3.94 (m, 2H), 3.86-3.79 (m, 3H), 3.74 | |
| | carboxamide | (br t, J = 5.6 Hz, 1H), 3.69 (br t, J = | |
| | | 5.6 Hz, 1H), 3.57-3.51 (m, 2H), 3.01- | |
| | | 2.91 (m, 4H), 2.84-2.77 (m, 3H), 2.73- | |
| | | 2.66 (m, 1H), 2.18 (br d, J = 6.4 Hz, | |
| | | 2H), 2.08-1.92 (m, 9H), 1.89-1.77 | |
| | | (m, 5H), 1.21 (s, 6H), 1.11 (s, 6H), | |
| | | 1.06-0.94 (m, 2H) | |
| 25 | 2-[4-[[4-[5-acetyl-3-[7-chloro-6-(1- | δ = 8.70 (s, 2H), 7.6-7.7 (m, 2H), 7.57 | 973.7 |
| | methylpyrazol-4-yl)-3,4-dihydro-2H- | (d, 1H, J = 8.8 Hz), 7.0-7.1 (m, 1H), | |
| | quinolin-1-yl]-6,7-dihydro-4H- | 6.97 (d, 1H, J = 2.4 Hz), 6.81 (dd, 1H, | |
| | pyrazolo[4,3-c]pyridin-1-yl]-1- | J = 2.4, 8.8 Hz), 6.56 (s, 1H), 5.93 (d, | |
| | piperidyl]methyl]-1-piperidyl]-N-[3- | 1H, J = 8.4 Hz), 4.85 (br d, 2H, J = 13.2 | |
| | (3-chloro-4-cyano-phenoxy)-2,2,4,4- | Hz), 4.31 (s, 1H), 4.1-4.2 (m, 2H), 4.04 | |
| | tetramethyl-cyclobutyl]pyrimidine-5- | (s, 1H), 3.9-4.0 (m, 5H), 3.76 (t, 1H, | |
| | carboxamide | J = 5.6 Hz), 3.6-3.7 (m, 2H), 2.9-3.1 (m, | |
| | | 4H), 2.7-2.9 (m, 4H), 2.2-2.3 (m, 4H), | |
| | | 2.1-2.2 (m, 2H), 2.0-2.1 (m, 4H), 1.8- | |
| | | 2.0 (m, 6H), 1.24 (d, 12H, J = 13.0 Hz), | |
| | | 1.1-1.2 (m, 2H) | |
| 26 | 2-[4-[[4-[5-acetyl-3-[7-methyl-6-(1- | δ = 8.70 (s, 2H), 7.62-7.51 (m, 2H), | 953.6 |
| | methylpyrazol-4-yl)-3,4-dihydro-2H- | 7.41-7.33 (m, 1H), 7.03-6.91 (m, | |
| | quinolin-1-yl]-6,7-dihydro-4H- | 2H), 6.81 (dd, J = 2.4, 8.8 Hz, 1H), | |
| | pyrazolo[4,3-c]pyridin-1-yl]-1- | 6.38 (s, 1H), 5.92 (d, J = 8.0 Hz, 1H), | |
| | piperidyl]methyl]-1-piperidyl]-N-[3- | 4.85 (br d, J = 12.8 Hz, 2H), 4.32- | |
| | (3-chloro-4-cyano-phenoxy)-2,2,4,4- | 4.02 (m, 4H), 3.98-3.88 (m, 5H), 3.78- | |
| | tetramethyl-cyclobutyl]pyrimidine-5- | 3.60 (m, 3H), 3.08-2.90 (m, 4H), | |
| | carboxamide | 2.86-2.71 (m, 4H), 2.38-2.23 (m, | |
| | | 6H), 2.22-2.10 (m, 4H), 2.08-2.00 | |
| | | (m, 4H), 1.90 (br d, J = 12.0 Hz, 5H), | |
| | | 1.24 (d, J = 12.8 Hz, 12H), 1.20-1.09 | |
| | | (m, 2H). | |
| 27 | 2-[4-[[4-[5-acetyl-3-[6-(1- | δ = 8.70 (s, 2H), 7.57 (d, J = 8.8 Hz, | 1007.5 |
| | methylpyrazol-4-yl)-7- | 1H), 7.53 (d, J = 3.6 Hz, 1H), 7.41 (d, J = | |
| | (trifluoromethyl)-3,4-dihydro-2H- | 6.4 Hz, 1H), 7.05 (br d, J = 17.2 Hz, | |
| | quinolin-1-yl]-6,7-dihydro-4H- | 1H), 6.98-6.87 (m, 2H), 6.81 (dd, J = | |
| | pyrazolo[4,3-c]pyridin-1-yl]-1- | 2.4, 8.8 Hz, 1H), 5.94 (brd, J = 8.0 Hz, | |
| | piperidyl]methyl]-1-piperidyl]-N-[3- | 1H), 4.86 (br d, J = 13.2 Hz, 2H), 4.32 | |
| | (3-chloro-4-cyano-phenoxy)-2,2,4,4- | (br s, 1H), 4.18-4.09 (m, 2H), 4.05 (s, | |
| | tetramethyl-cyclobutyl]pyrimidine-5- | 1H), 3.98-3.90 (m, 4H), 3.79-3.63 | |
| | carboxamide | (m, 3H), 3.03-2.68 (m, 7H), 2.43- | |
| | | 2.03 (m, 10H), 1.91 (brd, J = 12.8 Hz, | |
| | | 3H), 1.69 (br s, 5H), 1.24 (d, J = 14.4 | |
| | | Hz, 14H) | |
| 28 | 2-[4-[[4-[5-acetyl-3-[7- | δ = 8.71 (s, 2H), 7.58 (d, J = 8.8 Hz, | 1039.6 |
| | (difluoromethyl)-6-(1-methylpyrazol- | 1H), 7.53 (d, J = 5.6 Hz, 1H), 7.41 (d, J = | |
| | 4-yl)-3,4-dihydro-2H-quinolin-1-yl]- | 6.0 Hz, 1H), 7.09-6.95 (m, 2H), | |
| | 6,7-dihydro-4H-pyrazolo[4,3- | 6.90 (d, J = 13.2 Hz, 1H), 6.84-6.78 | |
| | c]pyridin-1-yl]-1-piperidyl]sulfonyl]- | (m, 1H), 6.71-6.36 (m, 1H), 6.02- | |
| | 1-piperidyl]-N-[3-(3-chloro-4-cyano- | 5.93 (m, 1H), 5.09-4.96 (m, 2H), 4.28 | |
| | phenoxy)-2,2,4,4-tetramethyl- | (s, 1H), 4.17-4.03 (m, 4H), 4.00- | |
| | cyclobutyl]pyrimidine-5-carboxamide | 3.86 (m, 6H), 3.80-3.64 (m, 3H), 3.29- | |
| | | 3.09 (m, 3H), 2.97 (m, 2H), 2.91- | |
| | | 2.84 (m, 2H), 2.80 (m, 1H), 2.73 (m, | |
| | | 1H), 2.33-2.14 (m, 5H), 2.12-1.97 | |
| | | (m, 6H), 1.88-1.71 (m, 2H), 1.27 (s, | |
| | | 6H), 1.23 (s, 6H) | |
| 64 | 2-[4-[2-[3-[5-acetyl-3-[7- | δ = 8.68 (s, 2H), 7.6-7.6 (m, 1H), 7.4- | 1004.5 |
| | (difluoromethyl)-6-(1-methylpyrazol- | 7.5 (m, 1H), 7.40 (d, 1H, J = 7.4 Hz), | |
| | 4-yl)-3,4-dihydro-2H-quinolin-1-yl]- | 6.9-7.1 (m, 3H), 6.81 (br d, 1H, J = 8.0 | |
| | 6,7-dihydro-4H-pyrazolo[4,3- | Hz), 6.3-6.7 (m, 1H), 6.0-6.2 (m, 1H), | |
| | c]pyridin-1-yl]pyrrolidin-1-yl]-2-oxo- | 4.6-4.9 (m, 1H), 4.3-4.5 (m, 1H), 4.1- | |
| | ethyl]piperazin-1-yl]-N-[3-(3-chloro- | 4.2 (m, 3H), 3.9-4.1 (m, 12H), 3.6-3.7 | |
| | 4-cyano-phenoxy)-2,2,4,4- | (m, 3H), 3.3-3.6 (m, 2H), 2.7-3.1 (m, | |
| | tetramethyl-cyclobutyl]pyrimidine-5- | 9H), 2.3-2.6 (m, 4H), 2.06 (d, 2H, | |
| | carboxamide | J = 6.0 Hz), 1.2-1.3 (m, 12H) | |
| 69 | 2-[4-[2-[4-[5-acetyl-3-[7- | δ = 8.76-8.69 (m, 2H), 7.63-7.52 (m, | 1033.6 |
| | (difluoromethyl)-6-(1-methylpyrazol- | 2H), 7.46-7.40 (m, 1H), 7.11-6.96 | |
| | 4-yl)-3,4-dihydro-2H-quinolin-1-yl]- | (m, 2H), 6.92-6.86 (m, 1H), 6.85- | |
| | 6,7-dihydro-4H-pyrazolo[4,3- | 6.79 (m, 1H), 6.73-6.37 (m, 1H), 6.06- | |
| | c]pyridin-1-yl]-1-piperidyl]-2-oxo- | 5.93 (m, 1H), 4.77-4.62 (m, 1H), | |
| | ethoxy]-1-piperidyl]-N-[3-(3-chloro- | 4.36-4.26 (m, 1H), 4.39-4.24 (m, | |
| | 4-cyano-phenoxy)-2,2,4,4- | 4H), 4.23-4.13 (m, 4H), 4.06 (s, 1H), | |
| | tetramethyl-cyclobutyl]pyrimidine-5- | 4.01-3.84 (m, 4H), 3.82-3.66 (m, | |
| | carboxamide | 4H), 3.65-3.55 (m, 2H), 3.24 (m, 1H), | |
| | | 2.95-2.80 (m, 4H), 2.76 (m, 1H), 2.31- | |
| | | 2.11 (m, 3H), 2.11-2.04 (m, 4H), | |
| | | 2.04-1.96 (m, 3H), 2.04-1.95 (m, | |
| | | 1H), 1.74-1.68 (m, 2H), 1.27 (s, 6H), | |
| | | 1.24 (s, 6H) | |
| 88 | 2-[2-[4-[5-acetyl-3-[7- | (DMSO-d6) δ = 9.12 (s, 2H), 8.24 (d, J = | 950.4 |
| | (difluoromethyl)-6-(1-methylpyrazol- | 8.8 Hz, 1H), 8.20 (s, 1H), 7.90 (d, J = | |
| | 4-yl)-3,4-dihydro-2H-quinolin-1-yl]- | 8.8 Hz, 1H), 7.75 (s, 1H), 7.49 (s, | |
| | 6,7-dihydro-4H-pyrazolo[4,3- | 1H), 7.22 (d, J = 2.0 Hz, 1H), 7.10 (s, | |
| | c]pyridin-1-yl]-1- | 1H), 7.04-6.99 (m, 1H), 6.96-6.62 | |
| | piperidyl]ethoxymethyl]-N-[3-(3- | (m, 2H), 4.73 (s, 2H), 4.30 (s, 1H), | |
| | chloro-4-cyano-phenoxy)-2,2,4,4- | 4.19-4.05 (m, 3H), 4.04-3.95 (m, | |
| | tetramethyl-cyclobutyl]pyrimidine-5- | 1H), 3.86 (s, 3H), 3.74-3.65 (m, 4H), | |
| | carboxamide | 3.61-3.55 (m, 2H), 3.05-2.96 (m, | |
| | | 2H), 2.84 (br d, J = 6.0 Hz, 3H), 2.76- | |
| | | 2.69 (m, 1H), 2.58 (br t, J = 6.0 Hz, | |
| | | 2H), 2.19-2.10 (m, 2H), 2.07 (s, 2H), | |
| | | 2.01-1.92 (m, 5H), 1.82 (br d, J = | |
| | | 11.2 Hz, 2H), 1.23 (s, 6H), 1.12 (s, 6H) | |
| 89 | 2-[2-[4-[5-acetyl-3-[7- | (DMSO-d6) δ = 8.97 (s, 2H), 8.14 (s, | 936.6 |
| | (difluoromethyl)-6-(1-methylpyrazol- | 1H), 8.05 (d, J = 9.2 Hz, 1H), 7.90 (d, | |
| | 4-yl)-3,4-dihydro-2H-quinolin-1-yl]- | J = 8.8 Hz, 1H), 7.75 (s, 1H), 7.49 (s, | |
| | 6,7-dihydro-4H-pyrazolo[4,3- | 1H), 7.22 (d, J = 2.0 Hz, 1H), 7.10 (s, | |
| | c]pyridin-1-yl]-1-piperidyl]ethoxy]- | 1H), 7.04-6.98 (m, 1H), 6.95-6.62 | |
| | N-[3-(3-chloro-4-cyano-phenoxy)- | (m, 2H), 4.51 (br t, J = 5.2 Hz, 2H), | |
| | 2,2,4,4-tetramethyl- | 4.30 (s, 1H), 4.19-4.09 (m, 2H), 4.08- | |
| | cyclobutyl]pyrimidine-5-carboxamide | 3.97 (m, 2H), 3.86 (s, 3H), 3.77- | |
| | | 3.65 (m, 2H), 3.62-3.55 (m, 2H), 3.05 | |
| | | (br d, J = 9.6 Hz, 2H), 2.84 (br d, J = | |
| | | 6.4 Hz, 3H), 2.80-2.65 (m, 3H), 2.23 | |
| | | (br t, J = 11.2 Hz, 2H), 2.07 (s, 2H), | |
| | | 2.02-1.93 (m, 5H), 1.88-1.80 (m, | |
| | | 2H), 1.23 (s, 6H), 1.11 (s, 6H) | |
| 92 | 2-[2-[4-[5-acetyl-3-[7- | (DMSO-d₆) δ = 9.04 (s, 2H), 8.22 (d, J = | 920.7 |
| | (difluoromethyl)-6-(1-methylpyrazol- | 9.6 Hz, 1H), 7.90 (d, J = 8.8 Hz, 1H), | |
| | 4-yl)-3,4-dihydro-2H-quinolin-1-yl]- | 7.74 (s, 1H), 7.49 (s, 1H), 7.22 (d, J = | |
| | 6,7-dihydro-4H-pyrazolo[4,3-c] | 2.0 Hz, 1H), 7.09 (s, 1H), 7.04-6.98 | |
| | pyridin-1-yl]-1-piperidyl]ethyl]-N-[3- | (m, 1H), 6.94-6.59 (m, 2H), 4.30 (s, | |
| | (3-chloro-4-cyano-phenoxy)-2,2,4,4- | 1H), 4.19-4.14 (m, 1H), 4.11 (s, 1H), | |
| | tetramethyl-cyclobutyl]pyrimidine-5- | 4.09-3.99 (m, 2H), 3.86 (s, 3H), 3.76- | |
| | carboxamide | 3.66 (m, 2H), 3.59-3.54 (m, 2H), | |
| | | 3.47-3.42 (m, 1H), 3.18-3.11 (m, | |
| | | 2H), 3.08-2.99 (m, 2H), 2.91-2.81 | |
| | | (m, 5H), 2.75-2.70 (m, 1H), 2.23- | |
| | | 2.12 (m, 2H), 2.07 (s, 2H), 1.98-1.91 | |
| | | (m, 4H), 1.88-1.78 (m, 2H), 1.23 (s, | |
| | | 6H), 1.12 (s, 6H), 1.05 (t, J = 6.8 Hz, 1H) | |
| 94 | 2-[[4-[5-acetyl-3-[7-(difluoromethyl)- | δ = 9.12-9.09 (m, 2H), 7.61-7.56 (m, | 906.6 |
| | 6-(1-methylpyrazol-4-yl)-3,4-dihydro- | 1H), 7.56-7.51 (m, 1H), 7.43-7.38 | |
| | 2H-quinolin-1-yl]-6,7-dihydro-4H- | (m, 1H), 7.06-6.99 (m, 1H), 6.98 (d, J = | |
| | pyrazolo[4,3-c]pyridin-1-yl]-1- | 2.4 Hz, 1H), 6.85 (s, 1H), 6.84-6.79 | |
| | piperidyl]methyl]-N-[3-(3-chloro-4- | (m, 1H), 6.75-6.36 (m, 1H), 6.28- | |
| | cyano-phenoxy)-2,2,4,4-tetramethyl- | 6.10 (m, 1H), 4.30-4.20 (m, 1H), 4.20- | |
| | cyclobutyl]pyrimidine-5-carboxamide | 4.19 (m, 1H), 4.17 (d, J = 8.4 Hz, | |
| | | 1H), 4.13-4.10 (m, 1H), 4.08 (s, 1H), | |
| | | 4.03-3.94 (m, 6H), 3.92-3.88 (m, | |
| | | 1H), 3.76-3.66 (m, 3H), 3.31-3.08 | |
| | | (m, 2H), 2.93-2.79 (m, 3H), 2.78- | |
| | | 2.73 (m, 1H), 2.54-2.27 (m, 4H), 2.16 | |
| | | (s, 1H), 2.11-2.02 (m, 4H), 2.02- | |
| | | 1.87 (m, 2H), 1.29 (s, 6H), 1.25 (s, 6H) | |
| 95 | 2-[4-[5-acetyl-3-[7-(difluoromethyl)- | δ = 8.72 (s, 2H), 7.61-7.50 (m, 2H), | 892.5 |
| | 6-(1-methylpyrazol-4-yl)-3,4-dihydro- | 7.43-7.37 (m, 1H), 7.07-6.95 (m, | |
| | 2H-quinolin-1-yl]-6,7-dihydro-4H- | 2H), 6.88-6.78 (m, 2H), 6.69-6.34 | |
| | pyrazolo[4,3-c]pyridin-1-yl]-1- | (m, 1H), 5.98-5.90 (m, 1H), 5.03 (m, | |
| | piperidyl]-N-[3-(3-chloro-4-cyano- | 2H), 4.30-4.17 (m, 2H), 4.17-4.09 | |
| | phenoxy)-2,2,4,4-tetramethyl- | (m, 2H), 4.05 (s, 1H), 3.98-3.90 (m, | |
| | cyclobutyl]pyrimidine-5-carboxamide | 4H), 3.77 (m, 1H), 3.71-3.63 (m, 2H), | |
| | | 3.23-3.09 (m, 2H), 2.92-2.72 (m, | |
| | | 4H), 2.27-2.14 (m, 3H), 2.13-1.97 | |
| | | (m, 6H), 1.24 (d, J = 16 Hz, 12H) | |
| 112 | 2-[4-[4-[5-acetyl-3-[7- | (DMSO-d₆): δ = 8.79 (s, 2H), 7.90 (d, | 1004.8 |
| | (difluoromethyl)-6-(1-methylpyrazol- | J = 8.8 Hz, 1H), 7.80-7.71 (m, 2H), | |
| | 4-yl)-3,4-dihydro-2H-quinolin-1-yl]- | 7.50 (s, 1H), 7.21 (d, J = 2.4 Hz, 1H), | |
| | 6,7-dihydro-4H-pyrazolo[4,3- | 7.10 (s, 1H), 7.00 (d, J = 2.4, 8.8 Hz, | |
| | c]pyridin-1-yl]piperidine-1- | 1H), 6.96-6.62 (m, 2H), 4.33-4.22 | |
| | carbonyl]piperazin-1-yl]-N-[3-(3- | (m, 2H), 4.19-4.10 (m, 2H), 4.04 (d, J = | |
| | chloro-4-cyano-phenoxy)-2,2,4,4- | 9.2 Hz, 1H), 3.86 (s, 7H), 3.77-3.66 | |
| | tetramethyl-cyclobutyl]pyrimidine-5- | (m, 4H), 3.61-3.55 (m, 2H), 3.29- | |
| | carboxamide | 3.23 (m, 4H), 3.00-2.90 (m, 2H), 2.89- | |
| | | 2.71 (m, 4H), 2.10-2.04 (m, 2H), | |
| | | 2.02 (s, 7H), 1.21 (s, 6H), 1.11 (s, 6H) | |
| 113 | 2-[4-[1-[4-[5-acetyl-3-[7- | δ = 8.70 (s, 2H), 7.62-7.51 (m, 2H), | 1003.6 |
| | (difluoromethyl)-6-(1-methylpyrazol- | 7.46-7.37 (m, 1H), 7.09-6.94 (m, | |
| | 4-yl)-3,4-dihydro-2H-quinolin-1-yl]- | 2H), 6.91-6.85 (m, 1H), 6.84-6.78 | |
| | 6,7-dihydro-4H-pyrazolo[4,3- | (m, 1H), 6.71-6.35 (m, 1H), 5.91 (d, J = | |
| | c]pyridin-1-yl]-1-piperidyl]ethyl]-1- | 8.0 Hz, 1H), 4.94-4.77 (m, 2H), | |
| | piperidyl]-N-[3-(3-chloro-4-cyano- | 4.27 (s, 1H), 4.19-4.10 (m, 2H), 4.04 | |
| | phenoxy)-2,2,4,4-tetramethyl- | (s, 1H), 3.96 (d, J = 2.0 Hz, 3H), 3.93- | |
| | cyclobutyl]pyrimidine-5-carboxamide | 3.80 (m, 2H), 3.78-3.67 (m, 3H), 2.99- | |
| | | 2.57 (m, 9H), 2.37-2.16 (m, 6H), | |
| | | 2.13-2.03 (m, 4H), 1.96-1.77 (m, | |
| | | 3H), 1.25 (s, 6H), 1.22 (s, 6H), 1.20- | |
| | | 1.06 (m, 2H), 0.98 (m, 3H) | |
| 114 | 6-[4-[4-[5-acetyl-3-[7- | (DMSO-d₆): δ = 8.24 (d, J = 9.2 Hz, | 1003.6 |
| | (difluoromethyl)-6-(1-methylpyrazol- | 1H), 7.93-7.88 (m, 1H), 7.86-7.81 | |
| | 4-yl)-3,4-dihydro-2H-quinolin-1-yl]- | (m, 1H), 7.78-7.73 (m, 1H), 7.50 (s, | |
| | 6,7-dihydro-4H-pyrazolo[4,3- | 1H), 7.38 (d, J = 10.0 Hz, 1H), 7.27- | |
| | c]pyridin-1-yl]piperidine-1-carbonyl]- | 7.24 (m, 1H), 7.13-7.09 (m, 1H), 7.05- | |
| | 1-piperidyl]-N-[3-(3-chloro-4-cyano- | 7.01 (m, 1H), 6.95-6.63 (m, 2H), | |
| | phenoxy)-2,2,4,4-tetramethyl- | 4.55-4.44 (m, 4H), 4.41-4.30 (m, | |
| | cyclobutyl]pyridazine-3-carboxamide | 1H), 4.23-4.09 (m, 3H), 4.04-3.96 | |
| | | (m, 1H), 3.86 (s, 3H), 3.79-3.67 (m, | |
| | | 2H), 3.62-3.53 (m, 2H), 3.26-3.22 | |
| | | (m, 1H), 3.18-3.05 (m, 3H), 2.92- | |
| | | 2.81 (m, 3H), 2.80-2.69 (m, 2H), 2.12- | |
| | | 2.07 (m, 2H), 2.02-1.88 (m, 6H), | |
| | | 1.80-1.70 (m, 3H), 1.65-1.50 (m, | |
| | | 2H), 1.25-1.20 (m, 6H), 1.14 (s, 6H) | |
| 115 | 6-[4-[4-[5-acetyl-3-[7- | 1.24 (d, J = 15.38 Hz, 12 H) 1.81-1.90 | 1002.6 |
| | (difluoromethyl)-6-(1-methylpyrazol- | (m, 4 H) 1.94-2.01 (m, 1 H) 2.02- | |
| | 4-yl)-3,4-dihydro-2H-quinolin-1-yl]- | 2.13 (m, 6 H) 2.18 (s, 1 H) 2.20-2.31 | |
| | 6,7-dihydro-4H-pyrazolo[4,3- | (m, 1 H) 2.74-2.92 (m, 6 H) 3.01- | |
| | c]pyridin-1-yl]piperidine-1-carbonyl]- | 3.10 (m, 2 H) 3.19-3.34 (m, 1 H) 3.63- | |
| | 1-piperidyl]-N-[3-(3-chloro-4-cyano- | 3.93 (m, 4 H) 3.96 (d, J = 1.88 Hz, 3 | |
| | phenoxy)-2,2,4,4-tetramethyl- | H) 4.05 (s, 1 H) 4.10-4.35 (m, 5 H) | |
| | cyclobutyl]pyridine-3-carboxamide | 4.39-4.52 (m, 2 H) 4.62-4.85 (m, 1 | |
| | | H) 6.06 (d, J = 8.13 Hz, 1 H) 6.37-6.58 | |
| | | (m, 1 H) 6.67-6.70 (m, 1 H) 6.81 (dd, | |
| | | J = 8.76, 2.38 Hz, 1 H) 6.86-6.91 (m, 1 | |
| | | H) 6.96-7.09 (m, 2 H) 7.38-7.44 (m, | |
| | | 1 H) 7.60 (s, 2 H) 7.92 (dd, J = 9.01, | |
| | | 2.50 Hz, 1 H) 8.50-8.62 (m, 1 H) | |
| 116 | 4-[4-[4-[5-acetyl-3-[7- | δ = 7.70 (d, J = 8.9 Hz, 2H), 7.62- | 1001.7 |
| | (difluoromethyl)-6-(1-methylpyrazol- | 7.53 (m, 2H), 7.45-7.40 (m, 1H), 7.10- | |
| | 4-yl)-3,4-dihydro-2H-quinolin-1-yl]- | 7.01 (m, 1H), 6.99 (d, J = 2.4 Hz, | |
| | 6,7-dihydro-4H-pyrazolo[4,3- | 1H), 6.94 (d, J = 9.0 Hz, 2H), 6.92- | |
| | c]pyridin-1-yl]piperidine-1-carbonyl]- | 6.88 (m, 1H), 6.83 (dd, J = 2.5, 8.8 Hz, | |
| | 1-piperidyl]-N-[3-(3-chloro-4-cyano- | 1H), 6.72-6.38 (m, 1H), 6.12 (d, J = | |
| | phenoxy)-2,2,4,4-tetramethyl- | 8.3 Hz, 1H), 4.87-4.69 (m, 1H), 4.21- | |
| | cyclobutyl]benzamide | 4.12 (m, 4H), 4.07 (s, 1H), 3.98 (d, J = | |
| | | 2.0 Hz, 3H), 3.91 (br d, J = 12.0 Hz, | |
| | | 3H), 3.83-3.65 (m, 3H), 3.35-3.19 | |
| | | (m, 1H), 2.97-2.72 (m, 8H), 2.35- | |
| | | 2.22 (m, 1H), 2.19 (s, 1H), 2.16-2.04 | |
| | | (m, 6H), 2.04-1.93 (m, 3H), 1.92- | |
| | | 1.81 (m, 2H), 1.26 (d, J = 17.4 Hz, | |
| | | 12H), 0.91 (br d, J = 7.4 Hz, 1H) | |
| 117 | 6-[4-[[4-[5-acetyl-3-[7- | δ = 8.16 (d, J = 9.2 Hz, 1H), 7.97 (d, | 989.6 |
| | (difluoromethyl)-6-(1-methylpyrazol- | J = 9.6 Hz, 1H), 7.59-7.52 (m, 2H), | |
| | 4-yl)-3,4-dihydro-2H-quinolin-1-yl]- | 7.41 (d, J = 6.0 Hz, 1H), 7.06-6.80 | |
| | 6,7-dihydro-4H-pyrazolo[4,3- | (m, 5H), 6.69-6.38 (m, 1H), 4.52 (d, J = | |
| | c]pyridin-1-yl]-1-piperidyl]methyl]-1- | 13.6 Hz, 2H), 4.32-3.99 (m, 5H), | |
| | piperidyl]-N-[3-(3-chloro-4-cyano- | 3.98-3.80 (m, 5H), 3.78-3.66 (m, | |
| | phenoxy)-2,2,4,4-tetramethyl- | 3H), 3.21-2.95 (m, 4H), 2.94-2.63 | |
| | cyclobutyl]pyridazine-3-carboxamide | (m, 5H), 2.34-1.85 (m, 16H), 1.29 (s, | |
| | | 6H), 1.21 (s, 6H). | |
| 118 | 2-[3-[4-[5-acetyl-3-[7- | 1.22 (s, 6 H) 1.26 (s, 6 H) 1.95-2.13 | 975.5 |
| | (difluoromethyl)-6-(1-methylpyrazol- | (m, 7 H) 2.13-2.28 (m, 3 H) 2.75 (br | |
| | 4-yl)-3,4-dihydro-2H-quinolin-1-yl]- | t, J = 5.50 Hz, 1 H) 2.77-2.96 (m, 4 H) | |
| | 6,7-dihydro-4H-pyrazolo[4,3- | 3.23 (br t, J = 12.69 Hz, 1 H) 3.64-3.84 | |
| | c]pyridin-1-yl]piperidine-1- | (m, 5 H) 3.96 (d, J = 2.25 Hz, 3 H) 4.05 | |
| | carbonyl]azetidin-1-yl]-N-[3-(3- | (s, 1 H) 4.10-4.29 (m, 4 H) 4.38- | |
| | chloro-4-cyano-phenoxy)-2,2,4,4- | 4.55 (m, 4 H) 4.65-4.79 (m, 1 H) 5.96 | |
| | tetramethyl-cyclobutyl]pyrimidine-5- | (d, J = 8.13 Hz, 1 H) 6.37-6.71 (m, 1 | |
| | carboxamide | H) 6.81 (dd, J = 8.69, 2.44 Hz, 1 H) | |
| | | 6.86-6.89 (m, 1 H) 6.96-7.07 (m, 2 | |
| | | H) 7.39-7.43 (m, 1 H) 7.52-7.60 (m, | |
| | | 2 H) 8.71 (s, 2 H). | |
| 119 | 2-[4-[3-[5-acetyl-3-[7- | δ = 8.70 (s, 2H), 7.5-7.6 (m, 2H), 7.42 | 975.7 |
| | (difluoromethyl)-6-(1-methylpyrazol- | (d, 1H, J = 6.4 Hz), 7.06 (d, 1H, J = 21.6 | |
| | 4-yl)-3,4-dihydro-2H-quinolin-1-yl]- | Hz), 6.9-7.0 (m, 2H), 6.81 (dd, 1H, | |
| | 6,7-dihydro-4H-pyrazolo[4,3- | J = 2.4, 8.8 Hz), 6.55 (dt, 1H, J = 11.1, | |
| | c]pyridin-1-yl]azetidine-1-carbonyl]- | 55.6 Hz), 5.96 (d, 1H, J = 8.4 Hz), 4.9- | |
| | 1-piperidyl]-N-[3-(3-chloro-4-cyano- | 5.0 (m, 1H), 4.7-4.9 (m, 3H), 4.60 (t, | |
| | phenoxy)-2,2,4,4-tetramethyl- | 1H, J = 8.4 Hz), 4.4-4.5 (m, 2H), 4.2-4.3 | |
| | cyclobutyl]pyrimidine-5-carboxamide | (m, 1H), 4.1-4.2 (m, 2H), 4.05 (s, 1H), | |
| | | 3.97 (d, 3H, J = 2.1 Hz), 3.6-4.0 (m, | |
| | | 4H), 3.0-3.2 (m, 2H), 2.8-3.0 (m, 2H), | |
| | | 2.7-2.8 (m, 2H), 2.4-2.6 (m, 1H), 2.0- | |
| | | 2.2 (m, 5H), 1.7-1.9 (m, 4H), 1.24 (d, | |
| | | 12H, J = 14.4 Hz) | |
| 120 | 2-[4-[4-[5-acetyl-3-[7-methyl-6-(1- | (400 MHz, DMSO-d₆): δ = 8.75 (s, | 966.6 |
| | methylpyrazol-4-yl)-3,4-dihydro-2H- | 2H), 7.89 (d, J = 7.6 Hz, 1H), 7.76- | |
| | quinolin-1-yl]-6,7-dihydro-4H- | 7.75 (m, 2H), 7.52 (s, 1H), 7.21 (s, | |
| | pyrazolo[4,3-c]pyridin-1- | 1H), 6.99-6.98 (m, 2H), 6.31 (s, 1H), | |
| | yl]piperidine-1-carbonyl]-1- | 4.75-4.73 (m, 2H), 4.50-4.49 (m, 1H), | |
| | piperidyl]-N-[3-(3-chloro-4-cyano- | 4.28-4.02 (m, 6H), 3.84 (s, 3H), 3.74- | |
| | phenoxy)-2,2,4,4-tetramethyl- | 3.71 (m, 2H), 3.53-3.52 (m, 3H), 3.18- | |
| | cyclobutyl]pyrimidine-5-carboxamide | 3.17 (m, 3H), 2.85-2.84 (m, 1H), 2.74- | |
| | | 2.73 (m, 4H), 2.15 (s, 3H), 2.07-2.06 | |
| | | (m, 2H), 1.95-1.91 (m, 6H), 1.73-1.72 | |
| | | (m, 3H), 1.50-1.49 (m, 2H), 1.21(s, 6 | |
| | | H), 1.10(s, 6 H). | |
| 122 | 2-[4-[4-[5-acetyl-3-[7- | (DMSO-d₆): δ = 8.75 (s, 2H), 7.89 (d, | 923.4 |
| | (difluoromethyl)-3,4-dihydro-2H- | J = 8.8 Hz, 1H), 7.71 (d, J = 9.2 Hz, | |
| | quinolin-1-yl]-6,7-dihydro-4H- | 1H), 7.21 (d, J = 2.4 Hz, 1H), 7.11 (d, | |
| | pyrazolo[4,3-c]pyridin-1-yl] | J = 7.2 Hz, 1H), 7.03-6.98 (m, 1H), | |
| | piperidine-1-carbonyl]-1-piperidyl]- | 6.97-6.65 (m, 2H), 6.65-6.60 (m, | |
| | N-[3-(3-chloro-4-cyano-phenoxy)- | 1H), 4.74 (br d, J = 12.4 Hz, 2H), 4.55- | |
| | 2,2,4,4-tetramethyl-cyclobutyl] | 4.43 (m, 1H), 4.40-4.31 (m, 1H), | |
| | pyrimidine-5-carboxamide | 4.28 (s, 1H), 4.21-4.07 (m, 3H), 4.03 | |
| | | (d, J = 8.8 Hz, 1H), 3.80-3.66 (m, | |
| | | 2H), 3.61-3.51 (m, 2H), 3.25-3.20 | |
| | | (m, 1H), 3.13-2.99 (m, 3H), 2.87 (br | |
| | | t, J = 6.0 Hz, 1H), 2.81 (br t, J = 5.6 | |
| | | Hz, 2H), 2.78-2.65 (m, 2H), 2.07 (s, | |
| | | 2H), 2.00-1.82 (m, 6H), 1.73 (br d, J = | |
| | | 12.2 Hz, 3H), 1.58-1.41 (m, 2H), | |
| | | 1.21 (s, 6H), 1.10 (s, 6H) | |
| 127 | 2-[4-[4-[5-acetyl-3-(6-cyano-3,4- | δ = 8.74-8.69 (m, 2H), 7.57 (d, J = | 898.4 |
| | dihydro-2H-quinolin-1-yl)-6,7- | 8.8 Hz, 1H), 7.31-7.27 (m, 1H), 7.25- | |
| | dihydro-4H-pyrazolo[4,3-c]pyridin-1- | 7.17 (m, 1H), 6.97 (d, J = 2.4 Hz, 1H), | |
| | yl]piperidine-1-carbonyl]-1- | 6.79-6.82 (m, 1H), 6.47-6.39 (m, | |
| | piperidyl]-N-[3-(3-chloro-4-cyano- | 1H), 5.98-5.99 (m, 1H), 4.87 (d, J = | |
| | phenoxy)-2,2,4,4-tetramethyl- | 13.2 Hz, 2H), 4.81-4.70 (m, 1H), 4.33- | |
| | cyclobutyl]pyrimidine-5-carboxamide | 4.26 (m, 1H), 4.22-4.09 (m, 4H), | |
| | | 4.05 (s, 1H), 3.98-3.88 (m, 1H), 3.81- | |
| | | 3.59 (m, 3H), 3.32-3.19 (m, 1H), | |
| | | 3.14-3.03 (m, 2H), 2.90-2.80 (m, | |
| | | 4H), 2.79-2.70 (m, 2H), 2.25-2.16 | |
| | | (m, 2H), 1.98 (s, 7H), 1.83 (s, 4H), | |
| | | 1.26 (s, 6H), 1.22 (s, 6H) | |
| 128 | 2-[4-[4-[5-acetyl-3-(3,4-dihydro-2H- | (DMSO-d6) δ = 8.76 (s, 2H), 8.14 (s, | 874.4 |
| | 1,6-naphthyridin-1-yl)-6,7-dihydro- | 1H), 8.06 (s, 1H), 7.98-7.93 (m, 1H), | |
| | 4H-pyrazolo[4,3-c]pyridin-1- | 7.90 (d, J = 8.8 Hz, 1H), 7.72 (d, J = | |
| | yl]piperidine-1-carbonyl]-1- | 9.2 Hz, 1H), 7.21 (d, J = 2.4 Hz, 1H), | |
| | piperidyl]-N-[3-(3-chloro-4-cyano- | 7.04-6.98 (m, 1H), 6.41-6.32 (m, | |
| | phenoxy)-2,2,4,4-tetramethyl- | 1H), 4.74 (br d, J = 12.8 Hz, 2H), 4.49 | |
| | cyclobutyl]pyrimidine-5-carboxamide | (br d, J = 11.2 Hz, 1H), 4.44-4.35 (m, | |
| | | 1H), 4.29 (s, 1H), 4.24-4.13 (m, 3H), | |
| | | 4.03 (d, J = 9.2 Hz, 1H), 3.81-3.71 | |
| | | (m, 2H), 3.65-3.58 (m, 2H), 3.23 (br | |
| | | s, 1H), 3.07 (br t, J = 11.6 Hz, 3H), | |
| | | 2.89 (br s, 1H), 2.82-2.74 (m, 3H), | |
| | | 2.73-2.66 (m, 1H), 2.09 (s, 2H), 2.03- | |
| | | 1.89 (m, 6H), 1.73 (br d, J = 11.2 Hz, | |
| | | 3H), 1.57-1.42 (m, 2H), 1.21 (s, 6H), | |
| | | 1.11 (s, 6H) | |
| 129 | 2-[4-[4-[5-acetyl-3-(3,4-dihydro-2H- | (DMSO-d6): δ = 8.76 (s, 2H), 7.90 (d, | 873.4 |
| | quinolin-1-yl)-6,7-dihydro-4H- | J = 8.8 Hz, 1H), 7.72 (d, J = 9.2 Hz, | |
| | pyrazolo[4,3-c]pyridin-1-yl] | 1H), 7.21 (d, J = 2.4 Hz, 1H), 7.06- | |
| | piperidine-1-carbonyl]-1-piperidyl]- | 6.95 (m, 2H), 6.95-6.85 (m, 1H), 6.60 | |
| | N-[3-(3-chloro-4-cyano-phenoxy)- | (t, J = 7.2 Hz, 1H), 6.45-6.34 (m, 1H), | |
| | 2,2,4,4-tetramethyl-cyclobutyl] | 4.74 (br d, J = 13.2 Hz, 2H), 4.55- | |
| | pyrimidine-5-carboxamide | 4.44 (m, 1H), 4.40-4.24 (m, 2H), 4.22- | |
| | | 4.12 (m, 1H), 4.11-3.98 (m, 3H), | |
| | | 3.81-3.64 (m, 2H), 3.58-3.48 (m, | |
| | | 2H), 3.26-3.16 (m, 1H), 3.14-3.01 | |
| | | (m, 3H), 2.89-2.65 (m, 5H), 2.07 (s, | |
| | | 2H), 2.01-1.83 (m, 6H), 1.74 (br d, J = | |
| | | 10.8 Hz, 3H), 1.59-1.42 (m, 2H), | |
| | | 1.21 (s, 6H), 1.11 (s, 6H) | |
| 150 | 2-[4-[[4-[5-acetyl-3-[7- | (DMSO-d6) δ = 8.74 (s, 2H), 8.14 (s, | 909.7 |
| | (difluoromethyl)-3,4-dihydro-2H- | 1H), 7.90 (d, J = 8.8 Hz, 1H), 7.70 (d, | |
| | quinolin-1-yl]-6,7-dihydro-4H- | J = 9.2 Hz, 1H), 7.21 (d, J = 2.4 Hz, | |
| | pyrazolo[4,3-c]pyridin-1-yl]-1- | 1H), 7.12 (d, J = 7.6 Hz, 1H), 7.04- | |
| | piperidyl]methyl]-1-piperidyl]-N-[3- | 6.98 (m, 1H), 6.98-6.59 (m, 3H), 4.73 | |
| | (3-chloro-4-cyano-phenoxy)-2,2,4,4- | (br d, J = 13.2 Hz, 2H), 4.29 (s, 1H), | |
| | tetramethyl-cyclobutyl]pyrimidine-5- | 4.15-3.98 (m, 4H), 3.78-3.65 (m, | |
| | carboxamide | 2H), 3.61-3.55 (m, 2H), 2.97 (br t, J = | |
| | | 11.2 Hz, 4H), 2.87-2.79 (m, 3H), 2.21 | |
| | | (br d, J = 6.8 Hz, 2H), 2.14-1.91 (m, | |
| | | 10H), 1.83 (br t, J = 11.2 Hz, 5H), 1.21 | |
| | | (s, 6H), 1.11 (s, 6H), 1.06-0.97 (m, 2H) | |
| 152 | 3-[4-[[4-[5-acetyl-3-[7- | (DMSO-d₆) δ = 8.71 (s, 1H), 8.11 (d, | 990.7 |
| | (difluoromethyl)-6-(1-methylpyrazol- | J = 9.2 Hz, 1H), 7.90 (d, J = 8.8 Hz, 1H), | |
| | 4-yl)-3,4-dihydro-2H-quinolin-1-yl]- | 7.75 (s, 1H), 7.50 (s, 1H), 7.24 (d, J = | |
| | 6,7-dihydro-4H-pyrazolo[4,3- | 2.4 Hz, 1H), 7.10 (s, 1H), 7.06-6.99 | |
| | c]pyridin-1-yl]-1-piperidyl\|methyl]-1- | (m, 1H), 6.95-6.63 (m, 2H), 4.96- | |
| | piperidyl]-N-[3-(3-chloro-4-cyano- | 4.53 (m, 2H), 4.44 (s, 1H), 4.24-4.10 | |
| | phenoxy)-2,2,4,4-tetramethyl- | (m, 2H), 4.07-3.96 (m, 2H), 3.87 (s, | |
| | cyclobutyl]-1,2,4-triazine-6- | 3H), 3.79-3.66 (m, 2H), 3.63-3.56 | |
| | carboxamide | (m, 2H), 3.17-3.05 (m, 2H), 2.99- | |
| | | 2.90 (m, 2H), 2.89-2.71 (m, 4H), 2.19 | |
| | | (br d, J = 6.0 Hz, 2H), 2.09-1.82 (m, | |
| | | 14H), 1.22 (s, 6H), 1.17-1.06 (m, 8H) | |
| 155 | 2-[4-[[4-[5-acetyl-3-(6-cyano-3,4- | δ = 8.70 (s, 2H), 7.57 (d, J = 8.8 Hz, | 884.6 |
| | dihydro-2H-quinolin-1-yl)-6,7- | 1H), 7.29 (s, 1H), 7.25-7.16 (m, 1H), | |
| | dihydro-4H-pyrazolo[4,3-c]pyridin-1- | 6.97 (d, J = 2.4 Hz, 1H), 6.88-6.72 | |
| | yl]-1-piperidyl]methyl]-1-piperidyl]- | (m, 1H), 6.49-6.36 (m, 1H), 5.94 (m, | |
| | N-[3-(3-chloro-4-cyano-phenoxy)- | 1H), 4.85 (m, 2H), 4.37-4.09 (m, 3H), | |
| | 2,2,4,4-tetramethyl- | 4.07-3.87 (m, 3H), 3.81-3.62 (m, | |
| | cyclobutyl]pyrimidine-5-carboxamide | 3H), 3.19-2.69 (m, 8H), 2.42-2.14 | |
| | | (m, 7H), 2.12-2.01 (m, 4H), 2.00- | |
| | | 1.73 (m, 6H), 1.25 (s, 6H), 1.22 (s, | |
| | | 6H), 1.20-1.13 (m, 2H) | |
| 157 | 2-[4-[[4-[5-acetyl-3-(3,4-dihydro-2H- | (DMSO-d6) δ = 8.81 (s, 2H), 8.21 (s, | 860.4 |
| | 1,6-naphthyridin-1-yl)-6,7-dihydro- | 1H), 8.10 (s, 1H), 8.02-7.94 (m, 2H), | |
| | 4H-pyrazolo[4,3-c]pyridin-1-yl]-1- | 7.76 (d, J = 9.2 Hz, 1H), 7.27 (d, J = | |
| | piperidyl]methyl]-1-piperidyl]-N-[3- | 2.4 Hz, 1H), 7.09-7.04 (m, 1H), 6.46- | |
| | (3-chloro-4-cyano-phenoxy)-2,2,4,4- | 6.36 (m, 1H), 4.80 (br d, J = 13.2 Hz, | |
| | tetramethyl-cyclobutyl]pyrimidine-5- | 2H), 4.35 (s, 1H), 4.28-4.23 (m, 2H), | |
| | carboxamide | 4.14-4.06 (m, 2H), 3.82 (br t, J = 5.8 | |
| | | Hz, 1H), 3.77 (br t, J = 5.4 Hz, 1H), | |
| | | 3.72-3.60 (m, 3H), 3.07-2.99 (m, | |
| | | 4H), 2.92 (br t, J = 5.2 Hz, 1H), 2.87- | |
| | | 2.81 (m, 2H), 2.27 (br d, J = 6.8 Hz, | |
| | | 2H), 2.18-2.00 (m, 9H), 1.96-1.83 | |
| | | (m, 5H), 1.27 (s, 6H), 1.17 (s, 6H), | |
| | | 1.13-1.03 (m, 2H) | |
| 158 | 2-[4-[[4-[5-acetyl-3-(3,4-dihydro-2H- | δ = 8.70 (s, 2H), 7.58 (d, J = 8.8 Hz, | 859.5 |
| | quinolin-1-yl)-6,7-dihydro-4H- | 1H), 7.08-6.88 (m, 3H), 6.87-6.77 | |
| | pyrazolo[4,3-c]pyridin-1-yl]-1- | (m, 1H), 6.70 (m, 1H), 6.50 (d, J = 7.6 | |
| | piperidyl]methyl]-1-piperidyl]-N-[3- | Hz, 1H), 5.93 (m, 1H), 4.95-4.78 (m, | |
| | (3-chloro-4-cyano-phenoxy)-2,2,4,4- | 2H), 4.04-4.03 (m, 1H), 4.30-4.02 | |
| | tetramethyl-cyclobutyl]pyrimidine-5- | (m, 3H), 3.89 (m, 2H), 3.87-3.48 (m, | |
| | carboxamide | 3H), 3.17-3.03 (m, 2H), 3.01-2.90 | |
| | | (m, 2H), 3.27-2.90 (m, 4H), 2.73 (m, | |
| | | 1H), 2.89-2.68 (m, 1H), 2.41-2.14 | |
| | | (m, 6H), 2.09-2.00 (m, 5H), 1.98- | |
| | | 1.85 (m, 4H), 1.25 (s, 6H), 1.22 (s, | |
| | | 6H), 1.20-1.15 (m, 2H) | |
| 160 | 2-[4-[2-[3-[5-acetyl-3-[7-methyl-6-(1- | δ = 8.69 (s, 2H), 7.62-7.34 (m, 3H), | 1108.6 |
| | methylpyrazol-4-yl)-3,4-dihydro-2H- | 7.06-6.93 (m, 2H), 6.81 (dd, J = 2.4, | |
| | quinolin-1-yl]-6,7-dihydro-4H- | 8.8 Hz, 1H), 6.42 (d, J = 2.0 Hz, 1H), | |
| | pyrazolo[4,3-c]pyridin-1-yl]azetidin- | 5.93 (d, J = 8.0 Hz, 1H), 4.92-4.77 | |
| | 1-yl]ethyl]-1-piperidyl]-N-[3-(3- | (m, 3H), 4.27-3.99 (m, 5H), 3.95 (d, J = | |
| | chloro-4-cyano-phenoxy)-2,2,4,4- | 2.8 Hz, 4H), 3.88 (br t, J = 5.6 Hz, | |
| | tetramethyl-cyclobutyl]pyrimidine-5- | 1H), 3.77-3.60 (m, 5H), 2.95 (br t, J = | |
| | carboxamide | 12.4 Hz, 2H), 2.88-2.66 (m, 6H), 2.28- | |
| | | 2.20 (m, 3H), 2.16-2.01 (m, 6H), | |
| | | 1.84-1.79 (m, 2H), 1.68 (br dd, J = | |
| | | 5.2, 10.8 Hz, 1H), 1.48-1.37 (m, 2H), | |
| | | 1.23 (d, J = 13.2 Hz, 12H), 1.20-1.13 | |
| | | (m, 1H). | |
| 161 | 2-[4-[2-[3-[5-acetyl-3-[7- | δ = 8.70 (s, 2H), 7.63-7.51 (m, 2H), | 488.9 |
| | (difluoromethyl)-6-(1-methylpyrazol- | 7.42 (d, J = 5.6 Hz, 1H), 7.09-6.95 | [M/2] |
| | 4-yl)-3,4-dihydro-2H-quinolin-1-yl]- | (m, 3H), 6.81 (dd, J = 2.4, 8.8 Hz, 1H), | |
| | 6,7-dihydro-4H-pyrazolo[4,3- | 6.55 (dt, J = 8.4, 55.6 Hz, 1H), 5.93 (d, | |
| | c]pyridin-1-yl]azetidin-1- | J = 8.4 Hz, 1H), 5.01-4.81 (m, 1H), | |
| | yl]ethyl]piperazin-1-yl]-N-[3-(3- | 4.26 (s, 1H), 4.16-4.07 (m, 3H), 4.06- | |
| | chloro-4-cyano-phenoxy)-2,2,4,4- | 3.99 (m, 2H), 3.97-3.93 (m, 6H), | |
| | tetramethyl-cyclobutyl]pyrimidine-5- | 3.89 (br t, J = 5.6 Hz, 1H), 3.83-3.68 | |
| | carboxamide | (m, 5H), 2.99-2.84 (m, 4H), 2.79- | |
| | | 2.68 (m, 2H), 2.61-2.49 (m, 6H), 2.21- | |
| | | 2.00 (m, 6H), 1.24 (d, J = 13.2 Hz, 12H) | |
| 162 | 2-[4-[2-[3-[5-acetyl-3-[7- | δ = 8.69 (s, 2H), 7.60 7.48 (m, 2H), | 975.8 |
| | (difluoromethyl)-6-(1-methylpyrazol- | 7.42 (d, J = 5.6 Hz, 1H), 7.11-6.92 (m, | |
| | 4-yl)-3,4-dihydro-2H-quinolin-1-yl]- | 3H), 6.85-6.77 (m, 1H), 6.70-6.38 | |
| | 6,7-dihydro-4H-pyrazolo[4,3- | (m, 1H), 5.92 (d, J = 8.4 Hz, 1H), 4.87- | |
| | c]pyridin-1-yl]azetidin-1-yl]ethyl]-1- | 4.82 (m, 2H), 4.25 (s, 1H), 4.16-4.07 | |
| | piperidyl]-N-[3-(3-chloro-4-cyano- | (m, 2H), 4.04 (s, 1H), 4.04-3.95 (m, | |
| | phenoxy)-2,2,4,4-tetramethyl- | 4H), 3.94-3.84 (m, 2H), 3.76-3.61 | |
| | cyclobutyl]pyrimidine-5-carboxamide | (m, 4H), 2.98-2.71 (m, 7H), 2.63 (s, | |
| | | 3H), 2.17-2.02 (m, 5H), 1.81 (d, J = | |
| | | 12.8 Hz, 2H), 1.45-1.38 (m, 2H), 1.32- | |
| | | 1.10 (m, 15H). | |
| 163 | 2-[4-[2-[3-[5-acetyl-3-[7- | δ = 8.68 (s, 2H), 7.62-7.51 (m, 2H), | 989.6 |
| | (difluoromethyl)-6-(1-methylpyrazol- | 7.42 (d, J = 7.2 Hz, 1H), 7.06 (d, J = | |
| | 4-yl)-3,4-dihydro-2H-quinolin-1-yl]- | 22.0 Hz, 1H), 6.97 (d, J = 2.4 Hz, 1H), | |
| | 6,7-dihydro-4H-pyrazolo[4,3- | 6.90 (d, J = 4.8 Hz, 1H), 6.81 (dd, J = | |
| | c]pyridin-1-yl]azetidin-1-yl]-2-oxo- | 2.4, 8.8 Hz, 1H), 6.55 (dt, J = 11.2, | |
| | ethyl]-1-piperidyl]-N-[3-(3-chloro-4- | 55.6 Hz, 1H), 5.94 (br d, J = 8.4 Hz, | |
| | cyano-phenoxy)-2,2,4,4-tetramethyl- | 1H), 4.99-4.80 (m, 3H), 4.74-4.63 | |
| | cyclobutyl]pyrimidine-5-carboxamide | (m, 1H), 4.54-4.38 (m, 3H), 4.32- | |
| | | 4.09 (m, 3H), 4.05 (s, 1H), 3.96 (d, J = | |
| | | 2.4 Hz, 4H), 3.83-3.67 (m, 3H), 3.02- | |
| | | 2.85 (m, 4H), 2.81-2.67 (m, 2H), 2.25- | |
| | | 2.04 (m, 8H), 1.96-1.85 (m, 2H), | |
| | | 1.24 (d, J = 13.6 Hz, 14H) | |
| 165 | 2-[4-[2-[3-[5-acetyl-3-[2-fluoro-4-(1- | δ = 8.70 (s, 2 H) 7.65-7.91 (m, 2 H) | 918.5 |
| | methylpyrazol-4-yl)anilino]-6,7- | 7.53-7.60 (m, 2 H) 7.15-7.24 (m, 2 | |
| | dihydro-4H-pyrazolo[4,3-c]pyridin-1- | H) 6.97 (d, J = 2.40 Hz, 1 H) 6.81 (dd, | |
| | yl]azetidin-1-yl]-2-oxo- | J = 8.80, 2.40 Hz, 1 H) 6.02 (br t, | |
| | ethyl]piperazin-1-yl]-N-[3-(3-chloro- | J = 7.60 Hz, 1 H) 5.81-5.94 (m, 1 H) | |
| | 4-cyano-phenoxy)-2,2,4,4- | 4.89-5.01 (m, 1 H) 4.77-4.88 (m, 1 | |
| | tetramethyl-cyclobutyl]pyrimidine-5- | H) 4.60-4.71 (m, 1 H) 4.41-4.59 (m, | |
| | carboxamide | 3 H) 4.32 (s, 1 H) 4.14 (d, J = 8.00 Hz, 1 | |
| | | H) 4.06 (s, 1 H) 4.01 (br d, J = 3.20 Hz, | |
| | | 4 H) 3.94 (d, J = 1.60 Hz, 3 H) 3.91 (br | |
| | | s, 1 H) 3.70-3.79 (m, 1 H) 3.27 (br d, | |
| | | J = 6.40 Hz, 2 H) 2.75 (br d, J = 4.80 Hz, | |
| | | 5 H) 2.68 (br t, J = 5.60 Hz, 1 H) 2.14- | |
| | | 2.24 (m, 3 H) 1.27 (s, 6 H) 1.22 (s, 6 H). | |
| 169 | 2-[4-[2-[4-[5-acetyl-3-[7- | δ = 8.74-8.66 (m, 2H), 7.60-7.50 (m, | 1033.7 |
| | (difluoromethyl)-6-(1-methylpyrazol- | 2H), 7.40 (d, J = 6.4 Hz, 1H), 7.07- | |
| | 4-yl)-3,4-dihydro-2H-quinolin-1-yl]- | 6.94 (m, 2H), 6.90-6.77 (m, 2H), 6.71- | |
| | 6,7-dihydro-4H-pyrazolo[4,3- | 6.35 (m, 1H), 6.02-5.89 (m, 1H), | |
| | c]pyridin-1-yl]-1-piperidyl]propoxy]- | 4.29-4.17 (m, 3H), 4.16-4.09 (m, | |
| | 1-piperidyl]-N-[3-(3-chloro-4-cyano- | 2H), 4.05 (s, 1H), 4.00-3.93 (m, 4H), | |
| | phenoxy)-2,2,4,4-tetramethyl- | 3.92-3.86 (m, 1H), 3.77-3.55 (m, | |
| | cyclobutyl]pyrimidine-5-carboxamide | 7H), 3.54-3.43 (m, 1H), 3.24-2.92 | |
| | | (m, 3H), 2.91-2.78 (m, 3H), 2.77- | |
| | | 2.71 (m, 1H), 2.67-2.47 (m, 2H), 2.30- | |
| | | 2.13 (m, 4H), 2.11-1.96 (m, 6H), | |
| | | 1.95-1.84 (m, 3H), 1.60-1.36 (m, | |
| | | 3H), 1.25 (s, 6H), 1.22 (s, 6H) | |
| 170 | 2-[4-[2-[4-[5-acetyl-3-[7- | δ = 8.70 (d, J = 2.4 Hz, 2H), 7.57 (d, | 1019.7 |
| | (difluoromethyl)-6-(1-methylpyrazol- | J = 8.8 Hz, 1H), 7.53 (d, J = 6.0 Hz, 1H), | |
| | 4-yl)-3,4-dihydro-2H-quinolin-1-yl]- | 7.40 (d, J = 6.8 Hz, 1H), 7.07-6.98 | |
| | 6,7-dihydro-4H-pyrazolo[4,3- | (m, 1H), 6.96 (d, J = 2.4 Hz, 1H), 6.91- | |
| | c]pyridin-1-yl]-1-piperidyl]ethoxy]-1- | 6.83 (m, 1H), 6.80 (dd, J = 2.4, 8.8 | |
| | piperidyl]-N-[3-(3-chloro-4-cyano- | Hz, 1H), 6.69-6.36 (m, 1H), 6.05- | |
| | phenoxy)-2,2,4,4-tetramethyl- | 5.90 (m, 1H), 4.31-4.14 (m, 3H), 4.13- | |
| | cyclobutyl]pyrimidine-5-carboxamide | 3.99 (m, 3H), 3.95 (d, J = 2.0 Hz, | |
| | | 3H), 3.89 (br t, J = 5.6 Hz, 1H), 3.75- | |
| | | 3.67 (m, 4H), 3.66-3.54 (m, 3H), 3.32- | |
| | | 3.12 (m, 2H), 2.93-2.68 (m, 6H), | |
| | | 2.54-2.35 (m, 2H), 2.32-2.23 (m, | |
| | | 2H), 2.17-2.00 (m, 9H), 1.93 (ddd, J = | |
| | | 3.2, 6.4, 9.6 Hz, 2H), 1.64 (dtd, J = 4.0, | |
| | | 8.4, 12.4 Hz, 2H), 1.23 (d, J = 14.4 Hz, 12H). | |
| 171 | 2-[4-[2-[4-[5-acetyl-3-[7- | (DMSO-d₆): δ = 8.74 (s, 2H), 7.90 (d, | 1017.8 |
| | (difluoromethyl)-6-(1-methylpyrazol- | J = 8.8 Hz, 1H), 7.74 (s, 1H), 7.69 (br d, | |
| | 4-yl)-3,4-dihydro-2H-quinolin-1-yl]- | J = 9.2 Hz, 1H), 7.49 (s, 1H), 7.21 (d, J = | |
| | 6,7-dihydro-4H-pyrazolo[4,3-c] | 2.0 Hz, 1H), 7.10 (s, 1H), 7.04-6.97 | |
| | pyridin-1-yl]-1-piperidyl]propyl]-1- | (m, 1H), 6.95-6.61 (m, 2H), 4.71 (br | |
| | piperidyl]-N-[3-(3-chloro-4-cyano- | d, J = 12.4 Hz, 2H), 4.28 (s, 1H), 4.20- | |
| | phenoxy)-2,2,4,4-tetramethyl- | 4.10 (m, 2H), 4.08-3.95 (m, 2H), 3.86 | |
| | cyclobutyl]pyrimidine-5- | (s, 3H), 3.78-3.65 (m, 2H), 3.63- | |
| | carboxamide | 3.54 (m, 2H), 3.10-2.78 (m, 8H), 2.72 | |
| | | (br s, 1H), 2.14-1.66 (m, 13H), 1.55- | |
| | | 1.44 (m, 1H), 1.31-0.88 (m, 19H). | |
| 172 | 2-[4-[2-[4-[5-acetyl-3-[7- | (DMSO-d₆): δ = 8.73 (s, 2H), 7.90 (d, | 1017.4 |
| | (difluoromethyl)-6-(1-methylpyrazol- | J = 8.8 Hz, 1H), 7.75 (s, 1H), 7.69 (d, J = | |
| | 4-yl)-3,4-dihydro-2H-quinolin-1-yl]- | 9.2 Hz, 1H), 7.49 (s, 1H), 7.21 (d, J = | |
| | 6,7-dihydro-4H-pyrazolo[4,3- | 2.4 Hz, 1H), 7.10 (s, 1H), 7.00 (d, J = | |
| | c]pyridin-1-yl]-1-piperidyl]-2-oxo- | 2.3, 8.8 Hz, 1H), 6.95-6.62 (m, 2H), | |
| | ethyl]-1-piperidyl]-N-[3-(3-chloro-4- | 4.71 (d, J = 12.8 Hz, 2H), 4.55-4.46 | |
| | cyano-phenoxy)-2,2,4,4-tetramethyl- | (m, 1H), 4.39-4.26 (m, 2H), 4.15 (d, J = | |
| | cyclobutyl]pyrimidine-5-carboxamide | 19.2 Hz, 2H), 4.07-3.96 (m, 2H), | |
| | | 3.86 (s, 3H), 3.79-3.66 (m, 2H), 3.62- | |
| | | 3.54 (m, 2H), 3.24-3.11 (m, 1H), | |
| | | 3.04-2.93 (m, 2H), 2.89-2.80 (m, | |
| | | 3H), 2.79-2.64 (m, 2H), 2.32 (d, J = | |
| | | 7.2 Hz, 2H), 2.08 (s, 3H), 2.00-1.84 | |
| | | (m, 6H), 1.82-1.68 (m, 3H), 1.21 (s, | |
| | | 6H), 1.11 (s, 7H) | |
| 173 | 2-[4-[[4-[5-acetyl-3-[7- | (400 MHz, DMSO-d₆): δ = 8.76 (s, | 1004.7 |
| | (difluoromethyl)-6-(1-methylpyrazol- | 2H), 7.90 (d, J = 8.8 Hz, 1H), 7.77- | |
| | 4-yl)-3,4-dihydro-2H-quinolin-1-yl]- | 7.68 (m, 2H), 7.49 (s, 1H), 7.21 (d, J = | |
| | 6,7-dihydro-4H-pyrazolo[4,3- | 2.4 Hz, 1H), 7.09 (s, 1H), 7.04-6.97 | |
| | c]pyridin-1-yl]cyclohexyl]methoxy]- | (m, 1H), 6.95-6.58 (m, 2H), 4.29 (s, | |
| | 1-piperidyl]-N-[3-(3-chloro-4-cyano- | 1H), 4.24-4.14 (m, 3H), 4.12 (s, 1H), | |
| | phenoxy)-2,2,4,4-tetramethyl- | 4.06-3.93 (m, 2H), 3.86 (s, 3H), 3.80- | |
| | cyclobutyl]pyrimidine-5-carboxamide | 3.65 (m, 2H), 3.62-3.47 (m, 5H), | |
| | | 2.90-2.70 (m, 4H), 2.07 (s, 2H), 2.03- | |
| | | 1.73 (m, 12H), 1.63-1.51 (m, 1H), | |
| | | 1.50-1.39 (m, 2H), 1.25-1.05 (m, 15H) | |
| 174 | 2-[4-[2-[4-[5-acetyl-3-[7- | δ = 8.69 (s, 2H), 7.62-7.50 (m, 2H), | 1003.6 |
| | (difluoromethyl)-6-(1-methylpyrazol- | 7.44-7.37 (m, 1H), 7.08-6.95 (m, | |
| | 4-yl)-3,4-dihydro-2H-quinolin-1-yl]- | 2H), 6.91-6.84 (m, 1H), 6.81 (dd, J = | |
| | 6,7-dihydro-4H-pyrazolo[4,3- | 2.4, 8.8 Hz, 1H), 6.72-6.35 (m, 1H), | |
| | c]pyridin-1-yl]-1-piperidyl]ethyl]-1- | 5.93 (d, J = 8.0 Hz, 1H), 4.84 (br d, J = | |
| | piperidyl]-N-[3-(3-chloro-4-cyano- | 13.2 Hz, 2H), 4.30-4.09 (m, 3H), 4.04 | |
| | phenoxy)-2,2,4,4-tetramethyl- | (s, 1H), 3.98-3.88 (m, 5H), 3.82- | |
| | cyclobutyl]pyrimidine-5-carboxamide | 3.58 (m, 3H), 3.09 (br d, J = 10.8 Hz, | |
| | | 2H), 3.00-2.77 (m, 5H), 2. 75 (br t, J = | |
| | | 5.6 Hz, 1H), 2.53-2.41 (m, 2H), 2.32- | |
| | | 2.23 (m, 2H), 2.17-2.13 (m, 2H), 2.11- | |
| | | 2.04 (m, 4H), 1.95 (br d, J = 9.2 Hz, | |
| | | 4H), 1.88-1.78 (m, 3H), 1.53-1.45 | |
| | | (m, 2H), 1.25 (s, 6H), 1.22 (s, 6H), | |
| | | 1.20-1.14 (m, 1H) | |
| 175 | 2-[4-[2-[4-[5-acetyl-3-[7-chloro-6-(1- | δ = 8.69 (s, 2H), 7.70 (d, J = 6.3 Hz, | 1001.5 |
| | methylpyrazol-4-yl)-3,4-dihydro-2H- | 2H), 7.57 (d, J = 8.8 Hz, 1H), 7.15- | |
| | quinolin-1-yl]-6,7-dihydro-4H- | 7.03 (m, 1H), 6.96 (d, J = 2.4 Hz, 1H), | |
| | pyrazolo[4,3-c]pyridin-1-y]]-1- | 6.81 (dd, J = 2.4, 8.8 Hz, 1H), 6.54 (s, | |
| | piperidyl]-2-oxo-ethyl]-1-piperidyl]- | 1H), 5.94 (d, J = 8.0 Hz, 1H), 4.86 (br | |
| | N-[3-(3-chloro-4-cyano-phenoxy)- | d, J = 13.2 Hz, 2H), 4.77 (br d, J = 13.2 | |
| | 2,2,4,4-tetramethyl- | Hz, 1H), 4.36-4.02 (m, 6H), 3.97- | |
| | cyclobutyl]pyrimidine-5-carboxamide | 3.76 (m, 5H), 3.70-3.58 (m, 2H), 3.26- | |
| | | 3.14 (m, 1H), 2.99 (brt, J = 11.6 Hz, | |
| | | 2H), 2.88-2.71 (m, 5H), 2.34-2.27 | |
| | | (m, 2H), 2.26-2.16 (m, 3H), 2.11- | |
| | | 1.88 (m, 9H), 1.25 (s, 7H), 1.22 (s, 7H). | |
| 176 | 2-[4-[2-[4-[5-acetyl-3-[7-methyl-6-(1- | δ = 8.70 (s, 2 H) 7.58 (d, J = 8.80 Hz, 1 | 981.6 |
| | methylpyrazol-4-yl)-3,4-dihydro-2H- | H) 7.52-7.56 (m, 1 H) 7.35-7.40 (m, | |
| | quinolin-1-yl]-6,7-dihydro-4H- | 1 H) 6.94-7.03 (m, 2 H) 6.81 (dd, | |
| | pyrazolo[4,3-c]pyridin-1-yl]-1- | J = 8.80, 2.40 Hz, 1 H) 6.35-6.40 (m, 1 | |
| | piperidyl]-2-oxo-ethyl]-1-piperidyl]- | H) 5.93 (d, J = 8.40 Hz, 1 H) 4.87 (br d, | |
| | N-[3-(3-chloro-4-cyano-phenoxy)- | J = 12.80 Hz, 2 H) 4.70-4.80 (m, 1 H) | |
| | 2,2,4,4-tetramethyl- | 4.28 (s, 1 H) 4.10-4.16 (m, 3 H) 4.04 | |
| | cyclobutyl]pyrimidine-5-carboxamide | (s, 2 H) 3.93-3.96 (m, 3 H) 3.88- | |
| | | 3.93 (m, 1 H) 3.76 (br t, J = 5.20 Hz, 1 | |
| | | H) 3.58-3.70 (m, 2 H) 3.13-3.27 (m, | |
| | | 1 H) 3.00 (br t, J = 11.60 Hz, 2 H) 2.63- | |
| | | 2.91 (m, 5 H) 2.28-2.35 (m, 2 H) 2.23 | |
| | | (s, 3 H) 2.19 (br d, J = 12.80 Hz, 3 H) | |
| | | 2.08 (br d, J = 3.20 Hz, 1 H) 2.00-2.05 | |
| | | (m, 4 H) 1.89-1.98 (m, 3 H) 1.60- | |
| | | 1.67 (m, 2 H) 1.25 (s, 6 H) 1.22 (s, 6 H). | |
| 188 | 2-[[4-[[4-[5-acetyl-3-[7- | (DMSO-d₆): δ = 9.09 (s, 2H), 8.25 (d, | 987.4 |
| | (difluoromethyl)-6-(1-methylpyrazol- | J = 9.2 Hz, 1H), 8.11 (s, 1H), 7.89 (d, J = | |
| | 4-yl)-3,4-dihydro-2H-quinolin-1-yl]- | 8.8 Hz, 1H), 7.74 (s, 1H), 7.49 (s, | |
| | 6,7-dihydro-4H-pyrazolo[4,3- | 1H), 7.22 (d, J = 2.4 Hz, 1H), 7.09 (s, | |
| | c]pyridin-1-yl]-1- | 1H), 7.05-6.99 (m, 1H), 6.96-6.62 | |
| | piperidyl]methyl]triazol-1-yl]methyl]- | (m, 2H), 5.92 (s, 2H), 4.29 (s, 1H), | |
| | N-[3-(3-chloro-4-cyano-phenoxy)- | 4.20-4.10 (m, 2H), 4.08-3.97 (m, | |
| | 2,2,4,4-tetramethyl- | 2H), 3.86 (s, 3H), 3.76-3.66 (m, 2H), | |
| | cyclobutyl]pyrimidine-5-carboxamide | 3.64 (s, 2H), 3.61-3.55 (m, 2H), 3.02- | |
| | | 2.91 (m, 2H), 2.89-2.70 (m, 4H), | |
| | | 2.16 (br t, J = 11.6 Hz, 2H), 2.07 (s, | |
| | | 2H), 2.03-1.91 (m, 5H), 1.89-1.81 | |
| | | (m, 2H), 1.21 (s, 6H), 1.11 (s, 6H) | |

### Example 19: Synthesis of 5-[8-(5-acetyl-1-tetrahydropyran-4-yl-6,7-dihydro-4H-pyrazolo[4,3-c]pyridin-3-yl)-3-isoquinolyl]-N-[[5-[[3-(3-chloro-4-cyano-phenoxy)-2,2,4,4-tetramethyl-cyclobutyl]carbamoyl]pyrimidin-2-yl]methyl]pyrimidine-2-carboxamide (Compound 75)

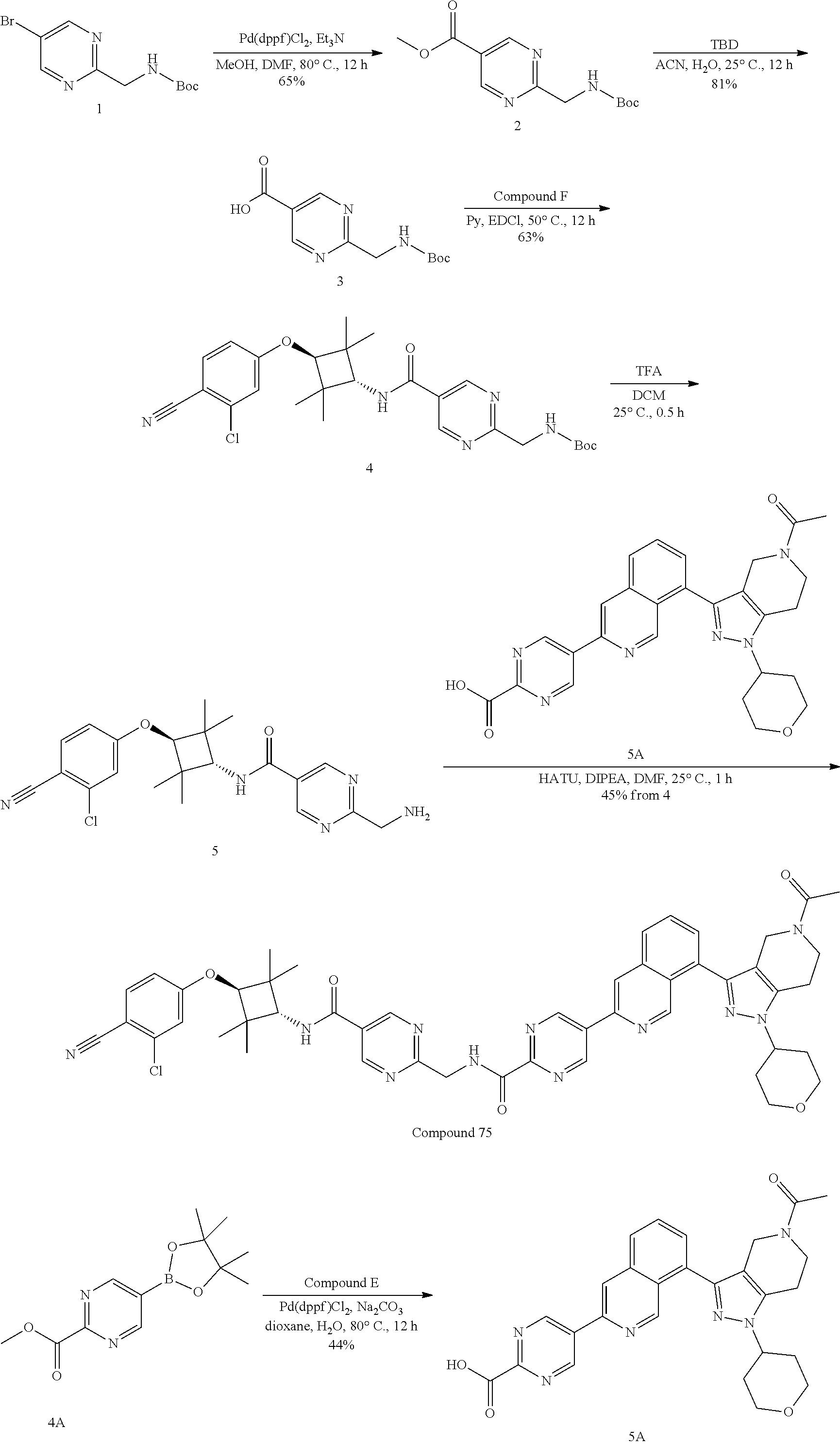

Synthesis of 3A was reported in US2018/99940 A1.

Synthesis of 4A was reported in EP3412669 A1.

To a solution of tert-butyl N-[(5-bromopyrimidin-2-yl)methyl]carbamate (500 mg, 1.74 mmol, 1.0 eq), Et₃N (878 mg, 8.68 mmol, 1.21 mL, 5.0 eq) in MeOH (5 mL), DMF (5 mL) was added Pd(dppf)Cl₂ (254 mg, 347 μmol, 0.2 eq) under N₂. The suspension was degassed and purged with CO for 3 times. The mixture was stirred under CO (50 Psi) at 80° C. for 12 h. The reaction mixture was quenched by addition water 100 mL, and then extracted with ethyl acetate (20 mL×3). The combined organic layers were washed with brine (20 mL×3), dried over anhydrous sodium sulfate, filtered and concentrated under reduced pressure to give a residue. The residue was purified by prep-TLC (SiO₂, Petroleum ether/Ethyl acetate=1:1). Compound methyl 2-[(tert-butoxycarbonylamino)methyl]pyrimidine-5-carboxylate (300 mg, 1.12 mmol, 65% yield) was obtained as a white solid.

¹H NMR (400 MHz, CHLOROFORM-d): δ=9.24 (s, 2H), 5.67 (br s, 1H), 4.68 (br d, J=4.8 Hz, 2H), 3.99 (s, 3H), 1.49 (s, 9H).

LC-MS: MS (ESI⁺): t_{R}=0.453 min, m/z=168.0 [M−100]

To a solution of tert-butyl N-[(5-bromopyrimidin-2-yl)methyl]carbamate (500 mg, 1.74 mmol, 1.0 eq), Et₃N (878 mg, 8.68 mmol, 1.21 mL, 5.0 eq) in MeOH (5 mL), DMF (5 mL) was added Pd(dppf)Cl₂ (254 mg, 347 μmol, 0.2 eq) under N₂. The suspension was degassed and purged with CO for 3 times. The mixture was stirred under CO (50 Psi) at 80° C. for 12 h. The reaction mixture was quenched by addition water 100 mL, and then extracted with ethyl acetate (20 mL×3). The combined organic layers were washed with brine (20 mL×3), dried over anhydrous sodium sulfate, filtered and concentrated under reduced pressure to give a residue. The residue was purified by prep-TLC (SiO₂, Petroleum ether/Ethyl acetate=1:1). Compound methyl 2-[(tert-butoxycarbonylamino)methyl]pyrimidine-5-carboxylate (300 mg, 1.12 mmol, 65% yield) was obtained as a white solid.

¹H NMR (400 MHz, CHLOROFORM-d): δ=9.24 (s, 2H), 5.67 (br s, 1H), 4.68 (br d, J=4.8 Hz, 2H), 3.99 (s, 3H), 1.49 (s, 9H).

LC-MS: MS (ESI⁺): t_{R}=0.453 min, m/z=168.0 [M−100]

To a solution of 2-[(tert-butoxycarbonylamino)methyl]pyrimidine-5-carboxylic acid (210 mg, 829 μmol, 1.0 eq), 4-(3-amino-2,2,4,4-tetramethyl-cyclobutoxy)-2-chloro-benzonitrile (325 mg, 829 μmol, 1.0 eq) in Py (5 mL) was added EDCI (476 mg, 2.49 mmol, 3.0 eq). The mixture was stirred at 50° C. for 12 h. The reaction mixture was concentrated in vacuo to give the crude product. The residue was purified by prep-TLC (SiO₂, Petroleum ether/Ethyl acetate=1:1). Compound tert-butyl N-[[5-[[3-(3-chloro-4-cyano-phenoxy)-2,2,4,4-tetramethyl-cyclobutyl]carbamoyl]pyrimidin-2-yl]methyl]carbamate (270 mg, 525.28 μmol, 63% yield) was obtained as a white solid.

¹H NMR (400 MHz, CHLOROFORM-d): δ=9.07 (s, 2H), 7.58 (d, J=9.6 Hz, 1H), 6.98 (d, J=2.4 Hz, 1H), 6.82 (dd, J=2.4, 8.4 Hz, 1H), 6.17 (br d, J=7.6 Hz, 1H), 5.63 (br s, 1H), 4.66 (br d, J=5.2 Hz, 2H), 4.17 (d, J=8.4 Hz, 1H), 4.08 (s, 1H), 1.49 (s, 9H), 1.27 (d, J=17.6 Hz, 12H).

LC-MS: MS (ESI⁺): t_{R}=0.597 min, m/z=458.1 [M−55]

To a solution of tert-butyl N-[[5-[[3-(3-chloro-4-cyano-phenoxy)-2,2,4,4-tetramethyl-cyclobutyl]carbamoyl]pyrimidin-2-yl]methyl]carbamate (45 mg, 87.55 μmol, 1.0 eq) in DCM (3 mL) was added TFA (767 mg, 6.73 mmol, 0.5 mL, 76 eq). The mixture was stirred at 25° C. for 0.5 h. The reaction mixture was concentrated in vacuo to give the crude product. Compound 2-(aminomethyl)-N-[3-(3-chloro-4-cyano-phenoxy)-2,2,4,4-tetramethyl-cyclobutyl]pyrimidine-5-carboxamide (42 mg) was obtained as a white solid and directly used in the next step without further purification.

LC-MS: MS (ESI⁺): t_{R}=0.473 min, m/z=414.1 [M+H⁺]

To a solution of 5-[8-(5-acetyl-1-tetrahydropyran-4-yl-6,7-dihydro-4H-pyrazolo[4,3-c]pyridin-3-yl)-3-isoquinolyl]pyrimidine-2-carboxylic acid (41 mg, 83.35 μmol, 1.0 eq), 2-(aminomethyl)-N-[3-(3-chloro-4-cyano-phenoxy)-2,2,4,4-tetramethyl-cyclobutyl]pyrimidine-5-carboxamide (42 mg, 83.35 μmol, 1.0 eq) in DMF (3 mL) was added DIPEA (53 mg, 416 μmol, 72 μL, 5.0 eq) and HATU (47 mg, 125 μmol, 1.5 eq). The mixture was stirred at 25° C. for 1 h. The reaction mixture was concentrated in vacuo to give the crude product. The residue was purified by prep-HPLC (FA condition; column: Welch Xtimate C18 150*25 mm*5 um; mobile phase: [water(FA)-ACN]; gradient: 55%-75% B over 15 min). Compound 5-[8-(5-acetyl-1-tetrahydropyran-4-yl-6,7-dihydro-4H-pyrazolo[4,3-c]pyridin-3-yl)-3-isoquinolyl]-N-[[5-[[3-(3-chloro-4-cyano-phenoxy)-2,2,4,4-tetramethyl-cyclobutyl]carbamoyl]pyrimidin-2-yl]methyl]pyrimidine-2-carboxamide (34.22 mg, 38.26 μmol, 45% yield over two steps) was obtained as a white solid.

¹H NMR (400 MHz, CHLOROFORM-d): δ=9.91 (s, 1H), 9.65 (d, J=4.8 Hz, 2H), 9.19-9.11 (m, 3H), 8.24 (d, J=9.6 Hz, 1H), 8.03 (s, 1H), 7.97 (dd, J=8.8, 14.4 Hz, 1H), 7.83 (td, J=7.6, 11.6 Hz, 1H), 7.66 (dd, J=7.2, 18.0 Hz, 1H), 7.59 (d, J=8.8 Hz, 1H), 6.98 (d, J=2.4 Hz, 1H), 6.82 (dd, J=2.4, 8.8 Hz, 1H), 6.24 (br d, J=7.2 Hz, 1H), 5.10 (d, J=5.2 Hz, 2H), 4.68 (s, 1H), 4.52 (s, 1H), 4.38-4.26 (m, 1H), 4.19 (br d, J=8.0 Hz, 3H), 4.09 (s, 1H), 4.04 (br t, J=5.6 Hz, 1H), 3.87 (t, J=6.0 Hz, 1H), 3.59 (br t, J=12.4 Hz, 2H), 3.01-2.81 (m, 2H), 2.58-2.34 (m, 2H), 2.25-2.07 (m, 3H), 2.03-1.96 (m, 2H), 1.28 (d, J=17.6 Hz, 12H)

LC-MS: MS (ESI⁺): t_{R}=2.372 min, m/z=894.5 [M+H⁺]

### Preparation of 5A

A mixture of [8-(5-acetyl-1-tetrahydropyran-4-yl-6,7-dihydro-4H-pyrazolo[4,3-c]pyridin-3-yl)-3-isoquinolyl]trifluoromethanesulfonate (100 mg, 190 μmol, 1.0 eq), methyl 5-(4,4,5,5-tetramethyl-1,3,2-dioxaborolan-2-yl)pyrimidine-2-carboxylate (60 mg, 228 mol, 1.2 eq), Pd(dppf)Cl₂ (6.98 mg, 9.53 μmol, 0.05 eq), Na₂CO₃ (60 mg, 571 μmol, 3.0 eq) and in dioxane (3 mL), water (0.3 mL) was degassed and purged with N₂ for 3 times, and then the mixture was stirred at 80° C. for 12 h under N₂. The reaction mixture was concentrated in vacuo to give the crude product. The residue was purified by prep-TLC (SiO₂, dichloromethane:methanol=10:1). The residue was purified by prep-HPLC (HCl condition; column: Phenomenex luna C18 150*25 mm*10 um; mobile phase: [water(HCl)-ACN]; gradient: 15%-45% B over 10 min). Compound 5-[8-(5-acetyl-1-tetrahydropyran-4-yl-6,7-dihydro-4H-pyrazolo[4,3-c]pyridin-3-yl)-3-isoquinolyl]pyrimidine-2-carboxylic acid (42 mg, 84.25 μmol, 44% yield) was obtained as a white solid.

¹H NMR (400 MHz, DMSO-d₆): δ=10.06 (s, 1H), 9.73 (s, 2H), 8.83 (s, 1H), 8.09 (d, J=8.4 Hz, 1H), 8.01-7.92 (m, 1H), 7.83-7.67 (m, 1H), 4.59 (br s, 2H), 4.52-4.44 (m, 1H), 4.04-4.01 (m, 3H), 3.58-3.48 (m, 3H), 2.98 (br t, J=4.8 Hz, 1H), 2.86 (br t, J=5.6 Hz, 1H), 2.23-2.08 (m, 4H), 2.05-1.92 (m, 3H).

LC-MS: MS (ESI⁺): t_{R}=0.424 min, m/z=499.2 [M+H⁺]

The compounds below were prepared in a similar manner as described in Examples 12, 13, 14, and 19.

| | | | LC-MS |
|---|---|---|---|
| Compound | Name | ¹H NMR (CDCl₃) | [M + H⁺] |
| 72 | 2-[[5-[8-(5-acetyl-1- | δ = 9.7-10.0 (m, 1H), 9.09 (d, 2H, J = 3.6 | 866.5 |
| | tetrahydropyran-4-yl-6,7-dihydro- | Hz), 8.6-8.8 (m, 1H), 8.35 (ddd, 1H, | |
| | 4H-pyrazolo[4,3-c]pyridin-3-yl)- | J = 2.4, 8.6, 18.8 Hz), 8.02 (s, 1H), 7.9- | |
| | 3-isoquinolyl]-2- | 7.9 (m, 1H), 7.7-7.8 (m, 1H), 7.5-7.7 (m, | |
| | pyridyl]oxymethyl]-N-[3-(3- | 2H), 7.12 (d, 1H, J = 8.8 Hz), 6.97 (d, 1H, | |
| | chloro-4-cyano-phenoxy)-2,2,4,4- | J = 2.0 Hz), 6.81 (dd, 1H, J = 2.4, 8.8 Hz), | |
| | tetramethyl- | 6.1-6.3 (m, 1H), 5.77 (s, 2H), 4.67 (s, | |
| | cyclobutyl]pyrimidine-5- | 1H), 4.50 (s, 1H), 4.2-4.4 (m, 1H), 4.16 | |
| | carboxamide | (br d, 3H, J = 8.8 Hz), 4.0-4.1 (m, 2H), | |
| | | 3.85 (t, 1H, J = 5.6 Hz), 3.57 (br t, 2H, | |
| | | J = 12.4 Hz), 2.8-3.0 (m, 2H), 2.44 (dq, | |
| | | 2H, J = 4.7, 12.4 Hz), 2.22 (s, 1H), 2.08 | |
| | | (s, 1H), 1.97 (br d, 2H, J = 12.0 Hz), 1.26 | |
| | | (d, 13H, J = 15.6 Hz) | |
| 74 | 2-[2-[5-[8-(5-acetyl-1- | (DMSO-d6) δ = 9.99 (s, 1 H) 9.47 (s, 2 | 894.5 |
| | tetrahydropyran-4-yl-6,7-dihydro- | H) 8.72 (br s, 2 H) 8.63 (s, 1 H) 8.03 (d, | |
| | 4H-pyrazolo[4,3-c]pyridin-3-yl)- | J = 8.00 Hz, 1 H) 7.84-7.96 (m, 2 H) | |
| | 3-isoquinolyl]pyrimidin-2- | 7.60-7.76 (m, 2 H) 7.19 (d, J = 2.40 Hz, | |
| | yl]ethyl-methyl-amino]-N-[3-(3- | 1 H) 6.97 (dd, J = 8.80, 2.40 Hz, 1 H) | |
| | chloro-4-cyano-phenoxy)-2,2,4,4- | 4.57 (s, 2 H) 4.39-4.53 (m, 1 H) 4.23 | |
| | tetramethyl- | (s, 1 H) 4.18 (br t, J = 7.20 Hz, 2 H) 3.96- | |
| | cyclobutyl]pyrimidine-5- | 4.04 (m, 3 H) 3.79-3.88 (m, 2 H) 3.53 | |
| | carboxamide | (br t, J = 12.00 Hz, 2 H) 3.35-3.36 (m, 1 | |
| | | H) 3.28 (br s, 2 H) 3.17 (s, 3 H) 2.94- | |
| | | 3.00 (m, 1 H) 2.12-2.23 (m, 2 H) 2.11 | |
| | | (s, 2 H) 2.01 (s, 1 H) 1.95 (br dd, | |
| | | J = 7.20, 4.80 Hz, 2 H) 1.14 (s, 6 H) 1.05 | |
| | | (s, 6 H). | |
| 77 | 2-[3-[[5-[8-(5-acetyl-1- | δ = 9.89 (d, J = 10.8 Hz, 1H), 9.28 (br d, | 921.5 |
| | tetrahydropyran-4-yl-6,7-dihydro- | J = 10.4 Hz, 1H), 9.05 (s, 2H), 8.58 (td, | |
| | 4H-pyrazolo[4,3-c]pyridin-3-yl)- | J = 2.4, 8.0 Hz, 1H), 8.36-8.25 (m, | |
| | 3-isoquinolyl]pyridine-2- | 2H), 8.20 (d, J = 7.2 Hz, 1H), 7.94 (dd, | |
| | carbonyl]amino]propyl]-N-[3-(3- | J = 8.4, 12.0 Hz, 1H), 7.80 (q, J = 8.0 Hz, | |
| | chloro-4-cyano-phenoxy)-2,2,4,4- | 1H), 7.67-7.59 (m, 1H), 7.56 (dd, J = | |
| | tetramethyl- | 1.2, 8.8 Hz, 1H), 6.96 (s, 1H), 6.79 (br | |
| | cyclobutyl]pyrimidine-5- | d, J = 8.8 Hz, 1H), 6.22 (br d, J = 8.0 | |
| | carboxamide | Hz, 1H), 4.72-4.47 (m, 2H), 4.36-4.25 | |
| | | (m, 1H), 4.22-4.12 (m, 3H), 4.08-4.00 | |
| | | (m, 2H), 3.87 (br t, J = 5.6 Hz, 1H), 3.67- | |
| | | 3.53 (m, 4H), 3.21 (t, J = 7.2 Hz, 2H), | |
| | | 2.99-2.84 (m, 2H), 2.51-2.39 (m, 2H), | |
| | | 2.31-2.23 (m, 2H), 2.23-2.07 (m, 3H), | |
| | | 1.98 (br d, J = 13.2 Hz, 2H), 1.29-1.19 | |
| | | (m, 12H). | |
| 78 | 2-[2-[[5-[8-(5-acetyl-1- | δ = 9.86 (s, 1 H) 9.29 (dd, J = 7.20, 1.60 | 907.6 |
| | tetrahydropyran-4-yl-6,7-dihydro- | Hz, 1 H) 9.08 (s, 2 H) 8.70 (br d, J = 4.40 | |
| | 4H-pyrazolo[4,3-c]pyridin-3-yl)- | Hz, 1 H) 8.51-8.59 (m, 1 H) 8.29 (dd, | |
| | 3-isoquinolyl]pyridine-2- | J = 8.00, 3.20 Hz, 1 H) 8.16 (d, J = 10.80 | |
| | carbonyl]amino]ethyl]-N-[3-(3- | Hz, 1 H) 7.91 (dd, J = 14.00, 8.40 Hz, 1 | |
| | chloro-4-cyano-phenoxy)-2,2,4,4- | H) 7.79 (dt, J = 11.60, 7.60 Hz, 1 H) 7.59- | |
| | tetramethyl- | 7.66 (m, 1 H) 7.58 (d, J = 8.80 Hz, 1 H) | |
| | cyclobutyl]pyrimidine-5- | 6.98 (d, J = 2.40 Hz, 1 H) 6.81 (dd, | |
| | carboxamide | J = 8.80, 2.40 Hz, 1 H) 6.30-6.41 (m, 1 | |
| | | H) 4.67 (s, 1 H) 4.51 (s, 1 H) 4.25-4.37 | |
| | | (m, 1 H) 4.12-4.22 (m, 3 H) 3.98-4.11 | |
| | | (m, 4 H) 3.86 (br t, J = 5.60 Hz, 1 H) 3.58 | |
| | | (br t, J = 12.00 Hz, 2 H) 3.42 (br t, J = 6.00 | |
| | | Hz, 2 H) 2.83-2.99 (m, 2 H) 2.45 (br d, | |
| | | J = 12.00 Hz, 2 H) 2.05-2.26 (m, 3 H) | |
| | | 1.92-2.03 (m, 2 H) 1.30 (s, 6 H) 1.25 | |
| | | (s, 6 H). | |
| 79 | 2-[[[5-[8-(5-acetyl-1- | δ = 9.88 (d, J = 3.6 Hz, 1H), 9.39 (d, J = | 893.4 |
| | tetrahydropyran-4-yl-6,7-dihydro- | 6.8 Hz, 1H), 9.18-9.05 (m, 3H), 8.62 | |
| | 4H-pyrazolo[4,3-c]pyridin-3-yl)- | (d, J = 8.0 Hz, 1H), 8.36 (dd, J = 3.2, 8.0 | |
| | 3-isoquinolyl]pyridine-2- | Hz, 1H), 8.20 (d, J = 10.0 Hz, 1H), 7.94 | |
| | carbonyl]amino]methyl]-N-[3-(3- | (dd, J = 8.0, 13.2 Hz, 1H), 7.85 (s, 1H), | |
| | chloro-4-cyano-phenoxy)-2,2,4,4- | 7.69-7.54 (m, 2H), 6.98 (d, J = 2.4 Hz, | |
| | tetramethyl- | 1H), 6.81 (dd, J = 2.4, 8.8 Hz, 1H), 6.18 | |
| | cyclobutyl]pyrimidine-5- | (d, J = 7.2 Hz, 1H), 5.06 (d, J = 5.6 Hz, | |
| | carboxamide | 2H), 4.68 (s, 1H), 4.52 (s, 1H), 4.37- | |
| | | 4.26 (m, 1H), 4.18 (d, J = 8.0 Hz, 3H), | |
| | | 4.11-3.98 (m, 2H), 3.87 (t, J = 5.6 Hz, | |
| | | 1H), 3.59 (t, J = 12.4 Hz, 2H), 2.99- | |
| | | 2.93 (m, 1H), 2.88 (t, J = 5.2 Hz, 1H), | |
| | | 2.56-2.39 (m, 2H), 2.28-2.06 (m, 3H), | |
| | | 2.04-1.94 (m, 2H), 1.29 (s, 6H), 1.25 | |
| | | (s, 6H) | |
| 131 | N-[3-(3-chloro-4-cyano- | δ = 9.84-9.90 (m, 1 H) 9.29-9.37 (m, 1 | 482.4 |
| | phenoxy)-2,2,4,4-tetramethyl- | H) 9.13 (s, 1 H) 9.09 (s, 1 H) 8.50-8.58 | [M/2 + H⁺] |
| | cyclobutyl]-2-[[rac-(3S)-1-[5-[8- | (m, 1 H) 8.14-8.21 (m, 1 H) 7.88-8.07 | |
| | (5-acetyl-1-tetrahydropyran-4-yl- | (m, 2 H) 7.79 (dt, J = 12.00, 7.60 Hz, 1 | |
| | 6,7-dihydro-4H-pyrazolo[4,3- | H) 7.54-7.66 (m, 2 H) 6.97 (s, 1 H) | |
| | c]pyridin-3-yl)-3- | 6.80 (br d, J = 8.80 Hz, 1 H) 6.19-6.46 | |
| | isoquinolyl]pyridine-2- | (m, 1 H) 4.85-4.95 (m, 1 H) 4.75-4.83 | |
| | carbonyl]pyrrolidin-3- | (m, 1 H) 4.67 (s, 1 H) 4.52 (s, 1 H) 4.43 | |
| | yl]oxymethyl]pyrimidine-5- | (br d, J = 3.20 Hz, 1 H) 4.24-4.36 (m, 1 | |
| | carboxamide | H) 4.10-4.22 (m, 4 H) 4.01-4.09 (m, 3 | |
| | | H) 3.81-3.99 (m, 3 H) 3.58 (br t, | |
| | | J = 12.00 Hz, 2 H) 2.84-2.99 (m, 2 H) | |
| | | 2.38-2.53 (m, 2 H) 2.24-2.35 (m, 1 H) | |
| | | 2.22 (s, 1 H) 2.11-2.18 (m, 1 H) 2.09 | |
| | | (s, 2 H) 1.98 (br d, J = 12.80 Hz, 2 H) | |
| | | 1.27-1.32 (m, 6 H) 1.24 (br d, J = 5.20 | |
| | | Hz, 6 H). | |
| 137 | 2-[4-[[4-[8-(5-acetyl-1- | (DMSO-d₆): δ = 9.83 (s, 1H), 8.75 (s, | 922.5 |
| | tetrahydropyran-4-yl-6,7-dihydro- | 2H), 8.40 (s, 1H), 8.12 (d, J = 11.6 Hz, | |
| | 4H-pyrazolo[4,3-c]pyridin-3-yl)- | 2H), 7.93-7.86 (m, 2H), 7.83-7.76 (m, | |
| | 3-isoquinolyl]pyrazol-1- | 1H), 7.72 (d, J = 8.8 Hz, 1H), 7.61- | |
| | yl]methyl]-1-piperidyl]-N-[3-(3- | 7.48 (m, 1H), 7.21 (d, J = 2.4 Hz, 1H), | |
| | chloro-4-cyano-phenoxy)-2,2,4,4- | 7.00 (dd, J = 2.4, 8.8 Hz, 1H), 4.75 (br | |
| | tetramethyl- | d, J = 13.6 Hz, 2H), 4.61-4.53 (m, 2H), | |
| | cyclobutyl]pyrimidine-5- | 4.51-4.40 (m, 1H), 4.28 (s, 1H), 4.11 | |
| | carboxamide | (br d, J = 6.4 Hz, 2H), 4.03 (br d, J = 9.2 | |
| | | Hz, 3H), 3.87-3.77 (m, 2H), 3.52 (br t, | |
| | | J = 11.2 Hz, 2H), 3.06-2.79 (m, 5H), | |
| | | 2.31-2.00 (m, 6H), 2.00-1.90 (m, 2H), | |
| | | 1.70-1.60 (m, 2H), 1.23-1.08 (m, 13H) | |
| 138 | 2-[4-[2-[4-[8-(5-acetyl-1- | (DMSO-d6): δ = 9.81 (s, 1H), 8.71 (s, | 952.5 |
| | tetrahydropyran-4-yl-6,7-dihydro | 2H), 8.40 (s, 1H), 8.12 (d, J = 12.8 Hz, | |
| | 4H-pyrazolo[4,3-c]pyridin-3-yl)- | 2H), 7.92-7.84 (m, 2H), 7.82-7.75 (m, | |
| | 3-isoquinolyl]pyrazol-1- | 1H), 7.69 (br d, J = 9.2 Hz, 1H), 7.59- | |
| | yl]ethoxy]-1-piperidyl]-N-[3-(3- | 7.47 (m, 1H), 7.20 (d, J = 2.4 Hz, 1H), | |
| | chloro-4-cyano-phenoxy)-2,2,4,4- | 6.99 (dd, J = 2.4, 8.8 Hz, 1H), 4.55 (br s, | |
| | tetramethyl- | 2H), 4.50-4.41 (m, 1H), 4.34 (br t, J = | |
| | cyclobutyl]pyrimidine-5- | 5.2 Hz, 2H), 4.27 (s, 1H), 4.16-4.07 | |
| | carboxamide | (m, 2H), 4.01 (br d, J = 9.2 Hz, 3H), | |
| | | 3.89 (br t, J = 5.2 Hz, 2H), 3.86-3.78 | |
| | | (m, 2H), 3.66-3.59 (m, 1H), 3.53-3.45 | |
| | | (m, 4H), 2.98-2.81 (m, 2H), 2.18-2.01 | |
| | | (m, 5H), 1.95 (br d, J = 8.0 Hz, 2H), | |
| | | 1.83 (br d, J = 8.8 Hz, 2H), 1.44-1.34 | |
| | | (m, 2H), 1.21-1.06 (m, 12H). | |
| 139 | 2-[4-[2-[4-[8-(5-acetyl-1- | δ = 9.6-10.0 (m, 1H), 8.69 (s, 2H), 8.09 | 936.6 |
| | tetrahydropyran-4-yl-6,7-dihydro- | (s, 1H), 7.7-8.0 (m, 3H), 7.4-7.7 (m, | |
| | 4H-pyrazolo[4,3-c]pyridin-3-yl)- | 2H), 6.97 (d, 1H, J = 2.4 Hz), 6.81 (dd, | |
| | 3-isoquinolyl]pyrazol-1-yl]ethyl]- | 1H, J = 2.4, 8.6 Hz), 5.93 (d, 1H, J = 8.0 | |
| | 1-piperidyl]-N-[3-(3-chloro-4- | Hz), 4.86 (d, 2H, J = 12.8 Hz), 4.68 (s, | |
| | cyano-phenoxy)-2,2,4,4- | 1H), 4.51 (s, 1H), 4.2-4.4 (m, 3H), 4.1- | |
| | tetramethyl- | 4.2 (m, 3H), 4.0-4.1 (m, 2H), 3.85 (t, | |
| | cyclobutyl]pyrimidine-5- | 1H, J = 6.0 Hz), 3.57 (t, 2H, J = 12.0 Hz), | |
| | carboxamide | 2.8-3.0 (m, 4H), 2.4-2.5 (m, 2H), 2. 1-2.2 | |
| | | (m, 3H), 1.8-2.0 (m, 7H), 1.23 (d, 15H, | |
| | | J = 13.2 Hz) | |
| 140 | 2-[4-[[5-[8-(5-acetyl-1- | (DMSO-d₆) δ = 9.96 (s, 1H), 9.34 (d, J = | 467.4 |
| | tetrahydropyran-4-yl-6,7-dihydro- | 2.0 Hz, 1H), 8.74 (s, 2H), 8.57-8.48 | [M/2 + H⁺] |
| | 4H-pyrazolo[4,3-c]pyridin-3-yl)- | (m, 2H), 8.04 (d, J = 8.4 Hz, 1H), 7.91- | |
| | 3-isoquinolyl]-2-pyridyl]methyl]- | 7.85 (m, 2H), 7.73-7.61 (m, 2H), 7.41 | |
| | 1-piperidyl]-N-[3-(3-chloro-4- | (d, J = 8.0 Hz, 1H), 7.21 (d, J = 2.4 Hz, | |
| | cyano-phenoxy)-2,2,4,4- | 1H), 7.00 (dd, J = 2.4, 8.8 Hz, 1H), 4.74 | |
| | tetramethyl- | (d, J = 12.8 Hz, 2H), 4.57 (s, 2H), 4.53- | |
| | cyclobutyl]pyrimidine-5- | 4.43 (m, 1H), 4.28 (s, 1H), 4.03 (d, J = | |
| | carboxamide | 9.2 Hz, 3H), 3.89-3.78 (m, 2H), 3.53 (t, | |
| | | J = 11.6 Hz, 2H), 3.02-2.95 (m, 3H), | |
| | | 2.90-2.82 (m, 1H), 2.77 (d, J = 6.8 Hz, | |
| | | 2H), 2.20-2.10 (m, 5H), 2.03-1.94 (m, | |
| | | 3H), 1.72 (d, J = 11.2 Hz, 2H), 1.21 (s, | |
| | | 8H), 1.10 (s, 6H) | |
| 141 | 2-[4-[2-[5-[8-(5-acetyl-1- | δ = 9.23 (s, 1H), 8.55 (d, J = 8.3 Hz, | 947.8 |
| | tetrahydropyran-4-yl-6,7-dihydro- | 1H), 8.35 (s, 1H), 8.13 (s, 1H), 8.09 (s, | |
| | 4H-pyrazolo[4,3-c]pyridin-3-yl)- | 1H), 7.90 (d, J = 8.4 Hz, 1H), 7.85 (d, J = | |
| | 3-isoquinolyl]-2-pyridyl]ethyl]-1- | 8.9 Hz, 1H), 7.82-7.75 (m, 2H), 7.52 | |
| | piperidyl]-N-[3-(3-chloro-4- | (d, J = 6.4 Hz, 1H), 7.38 (d, J = 2.3 Hz, | |
| | cyano-phenoxy)-2,2,4,4- | 1H), 7.30 (d, J = 9.6 Hz, 1H), 7.27 (d, J = | |
| | tetramethyl- | 1.1 Hz, 1H), 7.19-7.10 (m, 2H), 4.59- | |
| | cyclobutyl]pyrimidine-5- | 4.49 (m, 1H), 4.45 (br d, J = 13.0 Hz, | |
| | carboxamide | 2H), 3.92 (s, 3H), 3.88-3.80 (m, 1H), | |
| | | 3.66 (s, 3H), 3.39 (s, 3H), 3.36-3.32 | |
| | | (m, 1H), 3.06-2.92 (m, 4H), 2.20 (br d, | |
| | | J = 6.8 Hz, 2H), 2.15-2.05 (m, 4H), | |
| | | 1.94-1.77 (m, 9H), 1.68-1.58 (m, 2H), | |
| | | 1.56-1.44 (m, 2H), 1.17-1.02 (m, 2H) | |
| 141 | 2-[4-[2-[5-[8-(5-acetyl-1- | δ = 9.23 (s, 1H), 8.55 (d, J = 8.3 Hz, | 474.4 |
| | tetrahydropyran-4-yl-6,7-dihydro- | 1H), 8.35 (s, 1H), 8.13 (s, 1H), 8.09 (s, | [M/2 + H⁺] |
| | 4H-pyrazolo[4,3-c]pyridin-3-yl)- | 1H), 7.90 (d, J = 8.4 Hz, 1H), 7.85 (d, J = | |
| | 3-isoquinolyl]-2-pyridyl]ethyl]-1- | 8.9 Hz, 1H), 7.82-7.75 (m, 2H), 7.52 | |
| | piperidyl]-N-[3-(3-chloro-4- | (d, J = 6.4 Hz, 1H), 7.38 (d, J = 2.3 Hz, | |
| | cyano-phenoxy)-2,2,4,4- | 1H), 7.30 (d, J = 9.6 Hz, 1H), 7.27 (d, J = | |
| | tetramethyl- | 1.1 Hz, 1H), 7.19-7.10 (m, 2H), 4.59- | |
| | cyclobutyl]pyrimidine-5- | 4.49 (m, 1H), 4.45 (br d, J = 13.0 Hz, | |
| | carboxamide | 2H), 3.92 (s, 3H), 3.88-3.80 (m, 1H), | |
| | | 3.66 (s, 3H), 3.39 (s, 3H), 3.36-3.32 | |
| | | (m, 1H), 3.06-2.92 (m, 4H), 2.20 (br d, | |
| | | J = 6.8 Hz, 2H), 2.15-2.05 (m, 4H), | |
| | | 1.94-1.77 (m, 9H), 1.68-1.58 (m, 2H), | |
| | | 1.56-1.44 (m, 2H), 1.17-1.02 (m, 2H) | |
| 142 | 2-[4-[2-[5-[8-(5-acetyl-1- | δ = 9.84 (d, J = 7.2 Hz, 1H), 9.25 (br s, | 481.5 |
| | tetrahydropyran-4-yl-6,7-dihydro- | 1H), 8.68 (s, 2H), 8.52-8.32 (m, 1H), | [M/2 + H⁺] |
| | 4H-pyrazolo[4,3-c]pyridin-3-yl)- | 8.11 (d, J = 10.4 Hz, 1H), 7.91 (dd, J = | |
| | 3-isoquinolyl]-2-pyridyl]propyl]- | 8.4, 14.0 Hz, 1H), 7.77 (td, J = 7.6, 11.2 | |
| | 1-piperidyl]-N-[3-(3-chloro-4- | Hz, 1H), 7.64-7.54 (m, 2H), 7.33 (brd, | |
| | cyano-phenoxy)-2,2,4,4- | J = 7.6 Hz, 1H), 6.97 (d, J = 2.4 Hz, | |
| | tetramethyl- | 1H), 6.80 (dd, J = 2.4, 8.8 Hz, 1H), 5.91 | |
| | cyclobutyl]pyrimidine-5- | (d, J = 8.0 Hz, 1H), 4.81 (br t, J = 12.4 | |
| | carboxam | Hz, 2H), 4.72-4.46 (m, 2H), 4.36-4.24 | |
| | | (m, 1H), 4.18 (br d, J = 11.2 Hz, 2H), | |
| | | 4.12 (d, J = 8.0 Hz, 1H), 4.06-4.00 (m, | |
| | | 2H), 3.86 (t, J = 5.6 Hz, 1H), 3.58 (br t, | |
| | | J = 12.0 Hz, 2H), 3.17 (br dd, J = 1.6, | |
| | | 4.4 Hz, 1H), 2.94 (br t, J = 5.6 Hz, 1H), | |
| | | 2.90-2.81 (m, 3H), 2.53-2.40 (m, 2H), | |
| | | 2.25-2.06 (m, 3H), 2.01-1.91 (m, 3H), | |
| | | 1.91-1.83 (m, 1H), 1.75 (br d, J = 13.2 | |
| | | Hz, 1H), 1.56-1.47 (m, 2H), 1.36 (d, J = | |
| | | 6.8 Hz, 3H), 1.23 (d, J = 12.4 Hz, | |
| | | 12H), 1.21-1.13 (m, 2H). | |
| 143 | 2-[4-[[5-[8-(5-acetyl-1- | δ = 9.97 (s, 1H), 9.35 (d, J = 2.0 Hz, | 482.4 |
| | tetrahydropyran-4-yl-6,7-dihydro | 1H), 8.75 (s, 2H), 8.66-8.54 (m, 2H), | [M/2] |
| | 4H-pyrazolo[4,3-c]pyridin-3-yl)- | 8.05 (d, J = 8.0 Hz, 1H), 7.95-7.83 (m, | |
| | 3-isoquinolyl]-2- | 2H), 7.74-7.55 (m, 3H), 7.21 (d, J = 2.4 | |
| | pyridyl]methoxymethyl]-1- | Hz, 1H), 7.00 (dd, J = 2.4, 8.8 Hz, 1H), | |
| | piperidyl]-N-[3-(3-chloro-4- | 4.78 (br d, J = 13.2 Hz, 2H), 4.64 (s, | |
| | cyano-phenoxy)-2,2,4,4- | 2H), 4.57 (br s, 2H), 4.52-4.43 (m, | |
| | tetramethyl- | 1H), 4.28 (s, 1H), 4.03 (br d, J = 9.2 Hz, | |
| | cyclobutyl]pyrimidine-5- | 3H), 3.89-3.77 (m, 2H), 3.53 (br t, J = | |
| | carboxamide | 12.0 Hz, 2H), 3.45 (d, J = 6.4 Hz, 2H), | |
| | | 3.08-2.94 (m, 3H), 2.85 (br t, J = 5.2 | |
| | | Hz, 1H), 2.21-2.08 (m, 4H), 2.06-1.93 | |
| | | (m, 4H), 1.89-1.79 (m, 2H), 1.26-1.15 | |
| | | (m, 8H), 1.10 (s, 6H) | |
| 144 | 2-[4-[3-[5-[8-(5-acetyl-1- | δ = 9.82 (d, 1H, J = 8.4 Hz), 9.23 (dd, 1H, | 488.6 |
| | tetrahydropyran-4-yl-6,7-dihydro- | J = 2.4, 3.6 Hz), 8.68 (s, 2H), 8.37 (dt, | [M/2 + H⁺] |
| | 4H-pyrazolo[4,3-c]pyridin-3-yl)- | 1H, J = 2.3, 7.8 Hz), 8.09 (d, 1H, J = 11.2 | |
| | 3-isoquinolyl]-2-pyridyl]butyl]-1- | Hz), 7.90 (dd, 1H, J = 8.2, 14.4 Hz), 7.76 | |
| | piperidyl]-N-[3-(3-chloro-4- | (td, 1H, J = 7.7, 11.6 Hz), 7.5-7.6 (m, | |
| | cyano-phenoxy)-2,2,4,4- | 2H), 7.3-7.3 (m, 1H), 6.96 (d, 1H, J = 2.4 | |
| | tetramethyl- | Hz), 6.80 (dd, 1H, J = 2.4, 8.8 Hz), 5.91 | |
| | cyclobutyl]pyrimidine-5- | (d, 1H, J = 8.0 Hz), 4.81 (d, 2H, J = 13.2 | |
| | carboxamide | Hz), 4.67 (s, 1H), 4.50 (s, 1H), 4.2-4.4 | |
| | | (m, 1H), 4.1-4.2 (m, 3H), 4.0-4.1 (m, | |
| | | 2H), 3.86 (t, 1H, J = 5.6 Hz), 3.58 (t, 2H, | |
| | | J = 11.6 Hz), 2.8-3.0 (m, 5H), 2.4-2.6 (m, | |
| | | 2H), 2.0-2.3 (m, 3H), 1.98 (d, 2H, | |
| | | J = 13.0 Hz), 1.7-1.9 (m, 4H), 1.5-1.6 (m, | |
| | | 2H), 1.36 (d, 3H, J = 6.8 Hz), 1.22 (d, | |
| | | 12H, J = 11.6 Hz), 1.0-1.2 (m, 3H) | |
| 179 | 2-[4-[2-[[5-[8-(5-acetyl-1- | δ = 9.88 (d, J = 4.8 Hz, 1H), 9.30 (dd, | 990.5 |
| | tetrahydropyran-4-yl-6,7-dihydro- | J = 1.6, 8.0 Hz, 1H), 8.70 (s, 2H), 8.59 | |
| | 4H-pyrazolo[4,3-c]pyridin-3-yl)- | (td, J = 2.0, 8.0 Hz, 1H), 8.33 (dd, J = | |
| | 3-isoquinolyl]pyridine-2- | 3.2, 8.0 Hz, 1H), 8.22-8.10 (m, 2H), | |
| | carbonyl]amino]ethyl]-1- | 7.93 (dd, J = 8.4, 13.6 Hz, 1H), 7.80 (td, | |
| | piperidyl]-N-[3-(3-chloro-4- | J = 8.0, 10.8 Hz, 1H), 7.68-7.54 (m, | |
| | cyano-phenoxy)-2,2,4,4- | 2H), 6.97 (d, J = 2.4 Hz, 1H), 6.81 (dd, J = | |
| | tetramethyl- | 2.4, 8.4 Hz, 1H), 5.93 (d, J = 8.4 Hz, | |
| | cyclobutyl]pyrimidine-5- | 1H), 4.87 (br d, J = 13.6 Hz, 2H), 4.68 | |
| | carboxamide | (s, 1H), 4.51 (s, 1H), 4.31 (br s, 1H), | |
| | | 4.22-4.10 (m, 3H), 4.08-3.99 (m, 2H), | |
| | | 3.87 (t, J = 5.6 Hz, 1H), 3.66-3.52 (m, | |
| | | 4H), 3.04-2.83 (m, 4H), 2.54-2.38 (m, | |
| | | 2H), 2.24-2.07 (m, 3H), 2.02-1.89 (m, | |
| | | 4H), 1.79-1.67 (m, 5H), 1.24 (d, J = | |
| | | 13.0 Hz, 12H) | |
| 180 | 2-[4-[[5-[8-(5-acetyl-1- | δ = 9.87 (d, J = 3.2 Hz, 1H), 9.32-9.25 | 482.0 |
| | tetrahydropyran-4-yl-6,7-dihydro- | (m, 1H), 8.73 (s, 2H), 8.63-8.57 (m, | [M − 479⁻] |
| | 4H-pyrazolo[4,3-c]pyridin-3-yl)- | 1H), 8.37-8.31 (m, 1H), 8.18 (d, J = | |
| | 3-isoquinolyl]pyridine-2- | 10.0 Hz, 1H), 8.13-8.06 (m, 1H), 7.98- | |
| | carbonyl]amino]-1-piperidyl]-N- | 7.89 (m, 1H), 7.83-7.75 (m, 1H), 7.65- | |
| | [3-(3-chloro-4-cyano-phenoxy)- | 7.56 (m, 2H), 6.97 (d, J = 2.4 Hz, 1H), | |
| | 2,2,4,4- | 6.84-6.79 (m, 1H), 5.96 (d, J = 8.0 Hz, | |
| | tetramethylcyclobutyl]pyrimidine- | 1H), 4.84 (d, J = 13.6 Hz, 2H), 4.67 (s, | |
| | 5-carboxamide | 1H), 4.51(s, 1H), 4.42-4.24 (m, 2H), | |
| | | 4.23-4.10 (m, 3H), 4.07-3.95 (m, 2H), | |
| | | 3.91-3.78 (m, 1H), 3.66-3.50 (m, 2H), | |
| | | 3.38-3.20 (m, 2H), 3.03-2.82 (m, 2H), | |
| | | 2.57-2.41 (m, 2H), 2.41-1.66 (m, 9H), | |
| | | 1.25 (d, J = 15.2 Hz, 12H). | |
| 215 | 2-[4-[[4-[8-(5-acetyl-1-methyl- | δ = 9.51-9.86 (m, 1 H) 8.99 (s, 1 H) 8.70 | 852.6 |
| | 6,7-dihydro-4H-pyrazolo[4,3- | (s, 2 H) 8.10 (s, 2 H) 7.78-7.91 (m, 2 | |
| | c]pyridin-3-yl)-3- | H) 7.73 (br t, J = 7.20 Hz, 1 H) 7.49- | |
| | isoquinolyl]pyrazol-1-yl\|methyl]- | 7.60 (m, 2 H) 6.95-6.99 (m, 1 H) 6.77- | |
| | 1-piperidyl]-N-[3-(3-chloro-4- | 6.85 (m, 1 H) 5.93 (br d, J = 7.60 Hz, 1 | |
| | cyano-phenoxy)-2,2,4,4- | H) 4.92 (br d, J = 13.20 Hz, 2 H) 4.63 (s, | |
| | tetramethyl- | 1 H) 4.46 (s, 1 H) 4.07-4.19 (m, 3 H) | |
| | cyclobutyl]pyrimidine-5- | 3.98-4.06 (m, 2 H) 3.90 (d, J = 3.60 Hz, | |
| | carboxamide | 3 H) 3.84 (t, J = 5.60 Hz, 1 H) 2.91-3.00 | |
| | | (m, 2 H) 2.80-2.91 (m, 2 H) 2.29-2.43 | |
| | | (m, 1 H) 2.21 (s, 1 H) 2.06 (s, 1 H) 1.76- | |
| | | 1.84 (m, 3 H) 1.29 (s, 1 H) 1.25 (s, 6 | |
| | | H) 1.22 (s, 6 H). | |
| 222 | 2-[4-[3-[4-[8-(5-acetyl-1- | δ = 9.78-9.61 (m, 1H), 8.74-8.68 (m, | 977.6 |
| | tetrahydropyran-4-yl-6,7-dihydro- | 2H), 8.26-8.15 (m, 1H), 8.10-8.02 (m, | |
| | 4H-pyrazolo[4,3-c]pyridin-3-yl)- | 1H), 7.86-7.75 (m, 2H), 7.74-7.64 (m, | |
| | 3-isoquinolyl]pyrazol-1- | 1H), 7.61-7.54 (m, 1H), 7.54-7.44 (m, | |
| | yl]pyrrolidin-1-yl]-1-piperidyl]- | 1H), 7.00-6.95 (m, 1H), 6.85-6.77 (m, | |
| | N-[3-(3-chloro-4-cyano- | 1H), 6.04-5.88 (m, 1H), 4.88 (d, J = 2.8 | |
| | phenoxy)-2,2,4,4-tetramethyl- | Hz, 2H), 4.67-4.43 (m, 2H), 4.35-4.22 | |
| | cyclobutyl]pyrimidine-5- | (m, 1H), 4.20-4.11 (m, 3H), 4.07-4.03 | |
| | carboxamide | (m, 1H), 4.03-3.98 (m, 1H), 3.91-3.78 | |
| | | (m, 1H), 3.64-3.48 (m, 2H), 3.24-3.00 | |
| | | (m, 4H), 2.99-2.74 (m, 3H), 2.54-2.38 | |
| | | (m, 3H), 2.37-2.26 (m, 1H), 2.22-2.20 | |
| | | (m, 1H), 2.16-1.88 (m, 6H), 1.62 (s, | |
| | | 4H), 1.26 (s, 6H), 1.22 (s, 6H). | |
| 231 | 2-[4-[2-[5-[8-(5-acetyl-1-methyl- | δ = 9.80 (d, J = 11.6 Hz, 1H), 9.29 (d, | 877.7 |
| | 6,7-dihydro-4H-pyrazolo[4,3- | J = 1.3 Hz, 1H), 8.72 (s, 2H), 8.58-8.40 | |
| | c]pyridin-3-yl)-3-isoquinolyl]-2- | (m, 1H), 8.15 (d, J = 9.4 Hz, 1H), 7.94 | |
| | pyridyl]ethyl]-1-piperidyl]-N-[3- | (dd, J = 8.2, 13.6 Hz, 1H), 7.85-7.74 | |
| | (3-chloro-4-cyano-phenoxy)- | (m, 1H), 7.68-7.55 (m, 2H), 7.39 (br d, | |
| | 2,2,4,4-tetramethyl- | J = 8.1 Hz, 1H), 6.99 (d, J = 2.4 Hz, | |
| | cyclobutyl]pyrimidine-5- | 1H), 6.82 (dd, J = 2.4, 8.8 Hz, 1H), 5.95 | |
| | carboxamide | (d, J = 8.4 Hz, 1H), 4.89 (br d, J = 13.8 | |
| | | Hz, 2H), 4.65 (s, 1H), 4.49 (s, 1H), 4.15 | |
| | | (d, J = 8.1 Hz, 1H), 4.08-4.00 (m, 2H), | |
| | | 3.94 (d, J = 3.4 Hz, 3H), 3.87 (t, J = 5.8 | |
| | | Hz, 1H), 3.08-2.95 (m, 4H), 2.95-2.89 | |
| | | (m, 2H), 2.86 (br t, J = 5.5 Hz, 1H), 2.24- | |
| | | 2.08 (m, 3H), 1.94 (br d, J = 12.3 Hz, | |
| | | 2H), 1.86-1.79 (m, 3H), 1.30 (br s, | |
| | | 1H), 1.25 (d, J = 13.9 Hz, 12H) | |

### Example 20: Synthesis of N-[3-(3-chloro-4-cyano-phenoxy)-2,2,4,4-tetramethyl-cyclobutyl]-2-[4-[5-[(4R)-4-methyl-2-oxo-1,3,4,5-tetrahydro-1,5-benzodiazepin-6-yl]-3-(1-methylpyrazol-4-yl)indazol-1-yl]-1-piperidyl]pyrimidine-5-carboxamide (Compound 60)

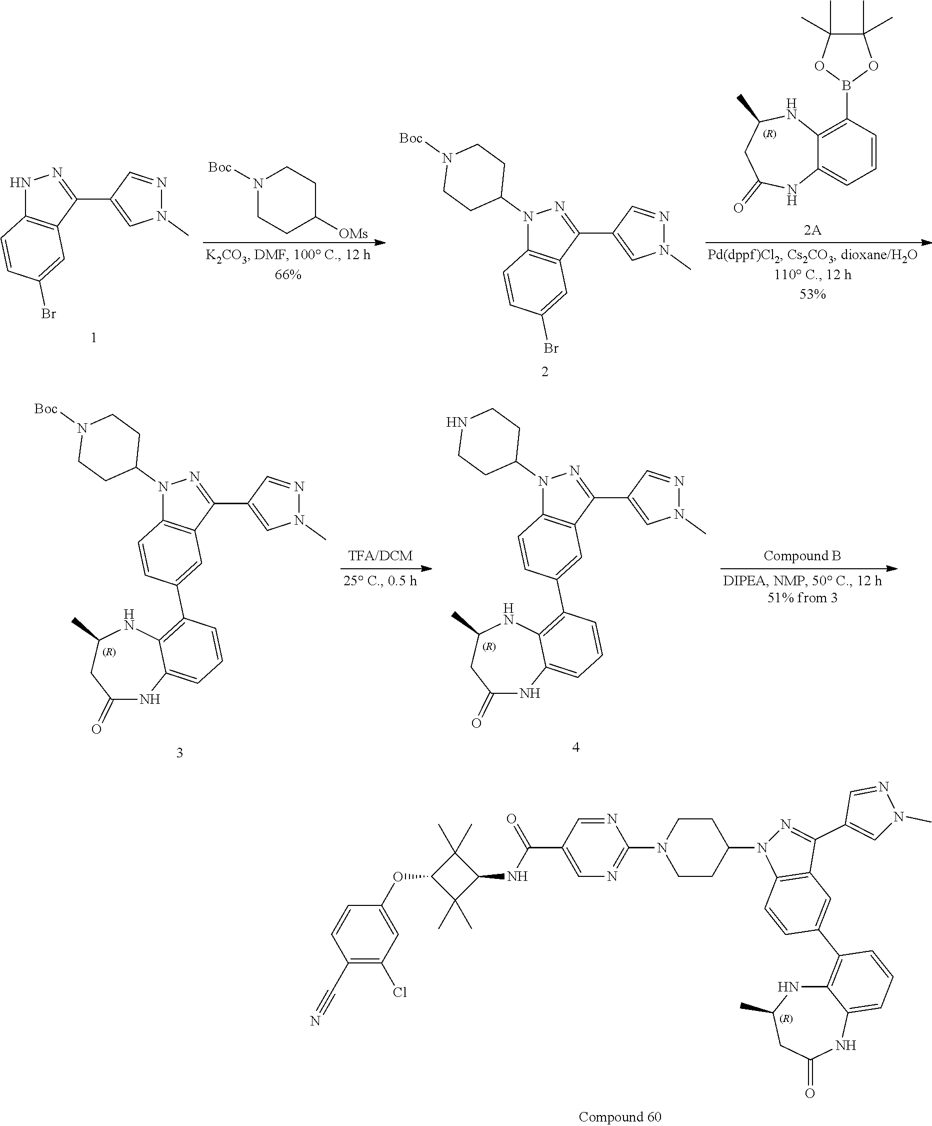

Synthesis of 1 was reported in WO2016/55028 A1.

Synthesis of 2A was reported in Medicinal Chemistry Letters, 2016, vol. 7, #5, p. 531-536

To a solution of 5-bromo-3-(1-methylpyrazol-4-yl)-1H-indazole (1 g, 3.61 mmol, 1.0 eq) and tert-butyl 4-methylsulfonyloxypiperidine-1-carboxylate (2.02 g, 7.22 mmol, 2.0 eq) in DMF (15 mL) was added K₂CO₃ (1.50 g, 10.83 mmol, 3.0 eq). The reaction mixture was stirred at 100° C. for 12 h. The reaction mixture was quenched by addition water (30 mL) at 0° C., and then diluted with water (30 mL) and extracted with ethyl acetate (50 mL×3). The combined organic layers were washed with brine (30 mL×3), dried over anhydrous sodium sulfate, filtered and concentrated under reduced pressure to give a residue. The residue was purified by prep-HPLC (FA condition: column: Phenomenex luna C18 150*40 mm*15 um; mobile phase: [water (FA)-ACN]; gradient: 55%-85% B over 15 min). The desired compound tert-butyl 4-[5-bromo-3-(1-methylpyrazol-4-yl)indazol-1-yl]piperidine-1-carboxylate (1.10 g, 2.39 mmol, 66% yield) was obtained as a white solid.

¹H NMR (400 MHz, CDCl₃): δ=8.03-7.94 (m, 2H), 7.86 (s, 1H), 7.46 (dd, J=1.6, 8.8 Hz, 1H), 7.33 (d, J=8.9 Hz, 1H), 4.61-4.47 (m, 1H), 4.32 (s, 2H), 4.01 (s, 3H), 2.97 (t, J=11.2 Hz, 2H), 2.26 (dq, J=4.4, 12.4 Hz, 2H), 2.07-1.95 (m, 2H), 1.50 (s, 9H).

LC-MS: MS (ESI⁺): t_{R}=0.604 min, m/z=460.1 [M+H⁺]

To a solution of tert-butyl 4-[5-bromo-3-(1-methylpyrazol-4-yl)indazol-1-yl]piperidine-1-carboxylate (200 mg, 434.44 mol, 1.0 eq) and (4R)-4-methyl-6-(4,4,5,5-tetramethyl-1,3,2-dioxaborolan-2-yl)-1,3,4,5-tetrahydro-1,5-benzodiazepin-2-one (144 mg, 477.88 μmol, 1.1 eq) in dioxane (5 mL) and H₂O (1 mL) was added Pd(dppf)Cl₂ (32 mg, 43.44 μmol, 0.1 eq) and Cs₂CO₃ (425 mg, 1.30 mmol, 3.0 eq). The reaction mixture was stirred at 110° C. for 12 h under N₂. The reaction mixture was filtered and concentrated under reduced pressure to give a residue. The desired compound tert-butyl 4-[5-[(4R)-4-methyl-2-oxo-1,3,4,5-tetrahydro-1,5-benzodiazepin-6-yl]-3-(1-methylpyrazol-4-yl)indazol-1-yl]piperidine-1-carboxylate (130 mg, 233.95 μmol, 53% yield) was obtained as light yellow solid.

LC-MS: MS (ESI⁺): t_{R}=0.568 min, m/z=556.3 [M+H⁺]

To a solution of tert-butyl 4-[5-[(4R)-4-methyl-2-oxo-1,3,4,5-tetrahydro-1,5-benzodiazepin-6-yl]-3-(1-methylpyrazol-4-yl)indazol-1-yl]piperidine-1-carboxylate (130 mg, 233.95 μmol, 1.0 eq) in DCM (5 mL) was added TFA (133 mg, 1.17 mmol, 86.89 μL, 5.0 eq). The reaction mixture was stirred at 25° C. for 0.5 h. The reaction mixture was concentrated under reduced pressure to give a residue. Compound (4R)-4-methyl-6-[3-(1-methylpyrazol-4-yl)-1-(4-piperidyl)indazol-5-yl]-1,3,4,5-tetrahydro-1,5-benzodiazepin-2-one (133 mg) was obtained as off-white solid and directly used in the next step without further purification.

LC-MS: MS (ESI⁺): t_{R}=0.406 min, m/z=456.2 [M+H⁺]

To a solution of (4R)-4-methyl-6-[3-(1-methylpyrazol-4-yl)-1-(4-piperidyl)indazol-5-yl]-1,3,4,5-tetrahydro-1,5-benzodiazepin-2-one (133 mg) and DIPEA (302 mg, 2.34 mmol, 406.73 μL, 10.0 eq) in NMP (2 mL) was added 2-chloro-N-[3-(3-chloro-4-cyano-phenoxy)-2,2,4,4-tetramethyl-cyclobutyl]pyrimidine-5-carboxamide (98 mg, 233.51 μmol, 1.0 eq). The reaction mixture was stirred at 50° C. for 12 h. The reaction mixture was filtered and concentrated under reduced pressure to give a residue. The residue was purified by prep-HPLC (FA condition: column: Welch Xtimate C18 150*25 mm*5 um; mobile phase: [water (FA)-ACN]; gradient: 58%-88% B over 15 min). The desired compound N-[3-(3-chloro-4-cyano-phenoxy)-2,2,4,4-tetramethyl-cyclobutyl]-2-[4-[5-[(4R)-4-methyl-2-oxo-1,3,4,5-tetrahydro-1,5-benzodiazepin-6-yl]-3-(1-methylpyrazol-4-yl)indazol-1-yl]-1-piperidyl]pyrimidine-5-carboxamide (103.17 mg, 121.24 μmol, 51% yield over two steps) was obtained as a off-white solid.

1H NMR (400 MHz, CDCl₃): δ=8.76 (s, 2H), 7.98 (s, 1H), 7.86 (s, 1H), 7.82 (s, 1H), 7.64-7.54 (m, 2H), 7.45-7.35 (m, 2H), 7.08 (dd, J=1.6, 7.2 Hz, 1H), 7.01-6.90 (m, 3H), 6.82 (dd, J=2.4, 8.8 Hz, 1H), 5.98 (d, J=8.0 Hz, 1H), 5.11 (d, J=13.2 Hz, 2H), 4.87-4.75 (m, 1H), 4.16 (d, J=8.0 Hz, 1H), 4.06 (s, 1H), 4.03-3.90 (m, 4H), 3.29 (t, J=11.6 Hz, 2H), 2.78 (dd, J=4.4, 13.6 Hz, 1H), 2.54-2.34 (m, 3H), 2.22 (d, J=10.0 Hz, 2H), 1.28 (s, 6H), 1.24 (s, 6H), 1.20 (d, J=6.4 Hz, 3H)

LC-MS: MS (ESI⁺): t_{R}=20.611 min, m/z=838.5 [M+H⁺]

The compounds below were prepared in a similar manner as described in Example 20.

| | | | LC-MS |
|---|---|---|---|
| Compound | Name | ¹H NMR (CDCl₃) | [M + H⁺] |
| 47 | N-[3-(3-chloro-4-cyano- | δ = 8.88 (s, 2H), 7.87-7.73 (m, 3H), | 799.3 |
| | phenoxy)-2,2,4,4-tetramethyl- | 7.58 (d, J = 8.8 Hz, 1H), 7.51 (s, 1H), | |
| | cyclobutyl]-2-[2-[3-(1- | 7.35-7.27 (m, 2H), 7.04 (dd, J = 1.6, 7.2 | |
| | methylpyrazol-4-yl)-5-[rac- | Hz, 1H), 6.97 (d, J = 2.4 Hz, 1H), 6.95-6.85 | |
| | (4R)-4-methyl-2-oxo-1,3,4,5- | (m, 2H), 6.81 (dd, J = 2.4, 8.8 Hz, | |
| | tetrahydro-1,5-benzodiazepin-6- | 1H), 6.11 (d, J = 8.0 Hz, 1H), 5.14-4.97 | |
| | yl]indazol-1- | (m, 2H), 4.94-4.81 (m, 2H), 4.15 (d, J = | |
| | yl]ethoxy]pyrimidine-5- | 8.0 Hz, 1H), 4.07 (s, 1H), 4.01 (s, 3H), | |
| | carboxamide | 3.95 (br d, J = 5.6 Hz, 1H), 3.67 (br d, | |
| | | J = 0.8 Hz, 1H), 2.76 (dd, J = 4.6, 13.6 Hz, | |
| | | 1H), 2.44 (dd, J = 6.3, 13.6 Hz, 1H), 1.28 | |
| | | (s, 6H), 1.24 (s, 6H), 1.20 (d, J = 6.2 Hz, | |
| | | 3H) | |
| 48 | N-[3-(3-chloro-4-cyano- | (DMSO-d₆) δ = 9.60 (s, 1H), 8.74 (s, | 812.6 |
| | phenoxy)-2,2,4,4-tetramethyl- | 2H), 8.39 (s, 1H), 7.9-8.0 (m, 3H), 7.71 | |
| | cyclobutyl]-2-[methyl-[2-[5- | (d, 1H, J = 9.2 Hz), 7.61 (d, 1H, J = 8.8 | |
| | [(4R)-4-methyl-2-oxo-1,3,4,5- | Hz), 7.35 (d, 1H, J = 9.6 Hz), 7.22 (d, 1H, | |
| | tetrahydro-1,5-benzodiazepin-6- | J = 2.4 Hz), 6.8-7.0 (m, 4H), 4.71 (t, 2H, | |
| | yl]-3-(1-methylpyrazol-4- | J = 6.4 Hz), 4.29 (s, 1H), 4.0-4.2 (m, 3H), | |
| | yl)indazol-1- | 3.8-3.9 (m, 4H), 3.74 (d, 1H, J = 2.4 Hz), | |
| | yl]ethyl]amino]pyrimidine-5- | 2.97 (s, 3H), 2.61 (dd, 1H, J = 4.4, 12.8 | |
| | carboxamide | Hz), 2.22 (dd, 1H, J = 6.0, 13.1 Hz), 1.1-1.2 | |
| | | (m, 12H), 1.05 (d, 3H, J = 6.0 Hz) | |
| 49 | N-[3-(3-chloro-4-cyano- | δ = 8.90 (s, 2H), 8.01-7.85 (m, 2H), | 813.6 |
| | phenoxy)-2,2,4,4-tetramethyl- | 7.79 (s, 1H), 7.58 (d, J = 8.8 Hz, 1H), | |
| | cyclobutyl]-2-[3-[5-[(4R)-4- | 7.54 (d, J = 8.8 Hz, 1H), 7.51 (s, 1H), | |
| | methyl-2-oxo-1,3,4,5- | 7.33 (dd, J = 1.6, 8.8 Hz, 1H), 7.05 (dd, | |
| | tetrahydro-1,5-benzodiazepin-6- | J = 2.0, 7.2 Hz, 1H), 6.99-6.90 (m, 3H), | |
| | yl]-3-(1-methylpyrazol-4- | 6.81 (dd, J = 2.4, 8.8 Hz, 1H), 6.09 (d, | |
| | yl)indazol-1- | J = 8.0 Hz, 1H), 4.67 (t, J = 6.4 Hz, 2H), | |
| | yl]propoxy]pyrimidine-5- | 4.49 (t, J = 6.0 Hz, 2H), 4.15 (d, J = 8.0 | |
| | carboxamide | Hz, 1H), 4.07 (s, 1H), 3.99 (s, 3H), 3.97-3.90 | |
| | | (m, 1H), 3.80-3.40 (m, 1H), 2.76 | |
| | | (dd, J = 4.4, 13.2 Hz, 1H), 2.55 (quin, J = | |
| | | 6.4 Hz, 2H), 2.46 (dd, J = 6.4, 13.2 Hz, | |
| | | 1H), 1.26 (d, J = 17.6 Hz, 12H), 1.17 (d, | |
| | | J = 6.4 Hz, 3H). | |
| 50 | N-[3-(3-chloro-4-cyano- | δ = 8.70 (s, 2H), 8.01 (s, 1H), 7.89 (s, | 826.5 |
| | phenoxy)-2,2,4,4-tetramethyl- | 1H), 7.80 (s, 1H), 7.58 (d, J = 8.8 Hz, | |
| | cyclobutyl]-2-[methyl-[3-[5- | 1H), 7.53-7.42 (m, 2H), 7.36 (dd, J = | |
| | [(4R)-4-methyl-2-oxo-1,3,4,5- | 1.2, 8.8 Hz, 1H), 7.07 (dd, J = 2.0, 7.2 | |
| | tetrahydro-1,5-benzodiazepin-6- | Hz, 1H), 6.99-6.90 (m, 3H), 6.81 (dd, | |
| | yl]-3-(1-methylpyrazol-4- | J = 2.4, 8.8 Hz, 1H), 5.94 (br d, J = 8.0 Hz, | |
| | yl)indazol-1- | 1H), 4.48 (t, J = 7.2 Hz, 2H), 4.13 (d, J = | |
| | yl]propyl]amino]pyrimidine-5- | 8.0 Hz, 1H), 4.04 (s, 1H), 4.00 (s, 3H), | |
| | carboxamide | 3.98-3.92 (m, 1H), 3.86 (br t, J = 7.2 | |
| | | Hz, 2H), 3.80-3.46 (m, 1H), 3.23 (s, | |
| | | 3H), 2.77 (dd, J = 4.4, 13.2 Hz, 1H), 2.47 | |
| | | (dd, J = 6.4, 13.6 Hz, 1H), 2.39 (quin, J = | |
| | | 6.8 Hz, 2H), 1.23 (d, J = 12.0 Hz, 12H), | |
| | | 1.18 (d, J = 6.4 Hz, 3H). | |
| 54 | N-[3-(3-chloro-4-cyano- | δ = 8.67 (s, 2H), 8.00 (s, 1H), 7.89 (s, | 1108.6 |
| | phenoxy)-2,2,4,4-tetramethyl- | 1H), 7.78 (s, 1H), 7.58 (dd, J = 2.4, 8.4 | |
| | cyclobutyl]-2-[4-[2-[5-[(4R)-4- | Hz, 2H), 7.40-7.31 (m, 2H), 7.09 (dd, | |
| | methyl-2-oxo-1,3,4,5- | J = 1.6, 7.2 Hz, 1H), 7.01-6.89 (m, 3H), | |
| | tetrahydro-1,5-benzodiazepin-6- | 6.81 (dd, J = 2.4, 8.8 Hz, 1H), 6.04 (d, | |
| | yl]-3-(1-methylpyrazol-4- | J = 8.0 Hz, 1H), 4.82-4.71 (m, 2H), 4.60 | |
| | yl)indazol-1-yl]ethoxymethyl]- | (t, J = 5.6 Hz, 2H), 4.14 (d, J = 8.0 Hz, | |
| | 1-piperidyl]pyrimidine-5- | 1H), 4.06 (s, 1H), 4.03-3.90 (m, 6H), | |
| | carboxamide | 3.71-3.54 (m, 1H), 3.33-3.21 (m, 2H), | |
| | | 2.85 (dt, J = 2.4, 13.2 Hz, 2H), 2.74 (dd, | |
| | | J = 4.4, 13.2 Hz, 1H), 2.46 (dd, J = 6.8, | |
| | | 13.6 Hz, 1H), 1.87-1.67 (m, 3H), 1.24 | |
| | | (d, J = 15.2 Hz, 12H), 1.16 (d, J = 6.4 | |
| | | Hz, 3H), 1.07 (dq, J = 4.0, 12.8 Hz, 2H). | |
| 55 | N-[3-(3-chloro-4-cyano- | δ = 8.71 (s, 2H), 8.00 (s, 1H), 7.89 (s, | 881.5 |
| | phenoxy)-2,2,4,4-tetramethyl- | 1H), 7.80 (s, 1H), 7.61-7.50 (m, 3H), | |
| | cyclobutyl]-2-[4-[3-[5-[(4R)-4- | 7.39 (dd, J = 1.1, 8.4 Hz, 1H), 7.07 (dd, | |
| | methyl-2-oxo-1,3,4,5- | J = 1.9, 7.2 Hz, 1H), 7.00-6.91 (m, 3H), | |
| | tetrahydro-1,5-benzodiazepin-6- | 6.80 (dd, J = 2.4, 8.8 Hz, 1H), 5.98 (d, | |
| | yl]-3-(1-methylpyrazol-4- | J = 8.0 Hz, 1H), 4.54 (br t, J = 6.4 Hz, 2H), | |
| | yl)indazol-1- | 4.13 (br d, J = 8.0 Hz, 2H), 4.05 (s, 2H), | |
| | yl]propyl]piperazin-1- | 4.03-3.97 (m, 4H), 3.97-3.92 (m, 1H), | |
| | yl]pyrimidine-5-carboxamide | 3.39 (t, J = 7.2 Hz, 1H), 2.92-2.66 (m, | |
| | | 7H), 2.52-2.26 (m, 5H), 1.33-1.11 (m, | |
| | | 16H) | |
| 56 | N-[3-(3-chloro-4-cyano- | δ = 8.71 (s, 2H), 8.00 (s, 1H), 7.89 (s, | 867.5 |
| | phenoxy)-2,2,4,4-tetramethyl- | 1H), 7.80 (s, 1H), 7.5-7.6 (m, 3H), 7.40 | |
| | cyclobutyl]-2-[4-[2-[5-[(4R)-4- | (d, 1H, J = 8.4 Hz), 7.07 (dd, 1H, J = 1.8, | |
| | methyl-2-oxo-1,3,4,5- | 7.2 Hz), 6.9-7.0 (m, 3H), 6.81 (dd, 1H, | |
| | tetrahydro-1,5-benzodiazepin-6- | J = 2.4, 8.8 Hz), 5.96 (d, 1H, J = 8.0 Hz), | |
| | yl]-3-(1-methylpyrazol-4- | 4.5-4.8 (m, 2H), 4.13 (d, 1H, J = 8.0 Hz), | |
| | yl)indazol-1-yl]ethyl]piperazin- | 4.05 (s, 1H), 3.9-4.0 (m, 7H), 3.0-3.2 (m, | |
| | 1-yl]pyrimidine-5-carboxamide | 2H), 2.6-2.9 (m, 5H), 2.46 (dd, 1H, | |
| | | J = 6.3, 13.2 Hz), 1.7-1.8 (m, 2H), 1.2-1.3 | |
| | | (m, 15H) | |
| 57 | N-[3-(3-chloro-4-cyano- | δ = 8.68 (s, 2 H) 7.67 (s, 1 H) 7.57 (d, | 856.6 |
| | phenoxy)-2,2,4,4-tetramethyl- | J = 8.8 Hz, 1 H) 7.50 (d, J = 8.4 Hz, 1 H) | |
| | cyclobuty1]-2-[4-[2-[3- | 7.37 (s, 1 H) 7.30 (m, 1 H) 7.08 (m, 1 H) | |
| | cyclopropyl-5-[(4R)-4-methyl- | 6.89-6.99 (m, 3 H) 6.81 (m, 1 H) 6.02 | |
| | 2-oxo-1,3,4,5-tetrahydro-1,5- | (d, J = 8.4 Hz, 1 H) 4.73-4.85 (m, 2 H) | |
| | benzodiazepin-6-yl]indazol-1- | 4.50 (t, J = 5.6 Hz, 2 H) 4.13 (d, J = 8.0 Hz, | |
| | yl]ethoxymethyl]-1- | 1 H) 4.05 (s, 1 H) 3.93-4.01 (m, 1 H) | |
| | piperidyl]pyrimidine-5- | 3.86 (t, J = 5.2 Hz, 2 H) 3.63 (m, 1 H) | |
| | carboxamide | 3.20-3.30 (m, 2 H) 2.86 (m, 2 H) 2.74 | |
| | | (m, 1 H) 2.46 (m, 1 H) 2.20 (m, 1 H) | |
| | | 1.73-1.87 (m, 1 H) 1.64-1.69 (m, 2 H) | |
| | | 1.25 (s, 6 H) 1.22 (s, 6 H) 1.18 (d, J = 6.4 | |
| | | Hz, 3 H) 1.06-1.13 (m, 2 H) 1.02-1.06 | |
| | | (m, 4 H) | |
| 58 | N-[3-(3-chloro-4-cyano- | δ = 8.70 (s, 2H), 8.02 (s, 1H), 7.90 (s, | 852.4 |
| | phenoxy)-2,2,4,4-tetramethyl- | 1H), 7.81 (s, 1H), 7.58 (d, 1H, J = 8.8 Hz), | |
| | cyclobutyl]-2-[4-[[5-[(4R)-4- | 7.4-7.5 (m, 2H), 7.37 (dd, 1H, J = 1.2, 8.6 | |
| | methyl-2-oxo-1,3,4,5- | Hz), 7.08 (dd, 1H, J = 1.8, 7.3 Hz), 6.9-7.0 | |
| | tetrahydro-1,5-benzodiazepin-6- | (m, 3H), 6.81 (dd, 1H, J = 2.4, 8.4 Hz), | |
| | yl]-3-(1-methylpyrazol-4- | 5.93 (d, 1H, J = 8.0 Hz), 4.92 (br d, 2H, | |
| | yl)indazol-1-yl]methyl]-1- | J = 13.2 Hz), 4.32 (d, 2H, J = 7.2 Hz), 4.13 | |
| | piperidyl]pyrimidine-5- | (d, 1H, J = 8.1 Hz), 3.9-4.1 (m, 5H), 2.94 | |
| | carboxamide | (t, 2H, J = 11.9 Hz), 2.78 (dd, 1H, J = 4.4, | |
| | | 13.3 Hz), 2.47 (dd, 2H, J = 6.6, 13.2 Hz), | |
| | | 1.80 (br d, 2H, J = 11.0 Hz), 1.39 (dq, 3H, | |
| | | J = 3.7, 12.4 Hz), 1.2-1.3 (m, 15H) | |
| 59 | N-[3-(3-chloro-4-cyano- | δ = 8.71 (s, 2 H) 7.69 (s, 1 H) 7.57 (d, | 841.6 |
| | phenoxy)-2,2,4,4-tetramethyl- | J = 8.4 Hz, 1 H) 7.55 (s, 1 H) 7.49 (d, | |
| | cyclobutyl]-2-[4-[3-[3- | J = 8.4 Hz, 1 H) 7.31 (m, 1 H) 7.07 (m, | |
| | cyclopropyl-5-[(4R)-4-methyl- | 1 H) 6.90-6.99 (m, 3 H) 6.81 (m, 1 H) | |
| | 2-oxo-1,3,4,5-tetrahydro-1,5- | 5.95 (d, J = 8.0 Hz, 1 H) 4.43 (t, J = 6.4 Hz, | |
| | benzodiazepin-6-yl]indazol-1- | 2 H) 4.13 (d, J = 8.0 Hz, 1 H) 4.05 (s, 1 H) | |
| | yl]propyl]piperazin-1- | 3.95-4.00 (m, 1 H) 3.88-3.95 (m, 4 H) | |
| | yl]pyrimidine-5-carboxamide | 2.78 (m, 1 H) 2.44-2.52 (m, 5 H) 2.40 | |
| | | (t, J = 6.4 Hz, 2 H) 2.12-2.24 (m, 3 H) | |
| | | 1.25 (s, 6 H) 1.22 (s, 6 H) 1.20 (s, 3 H) | |
| | | 1.02-1.09 (m, 4 H) | |

### Example 21: Synthesis of 2-[4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-1-piperidyl]-N-[3-(3-chloro-4-cyano-phenoxy)-2,2,4,4-tetramethyl-cyclobutyl]pyrimidine-5-carboxamide (Compound 136)

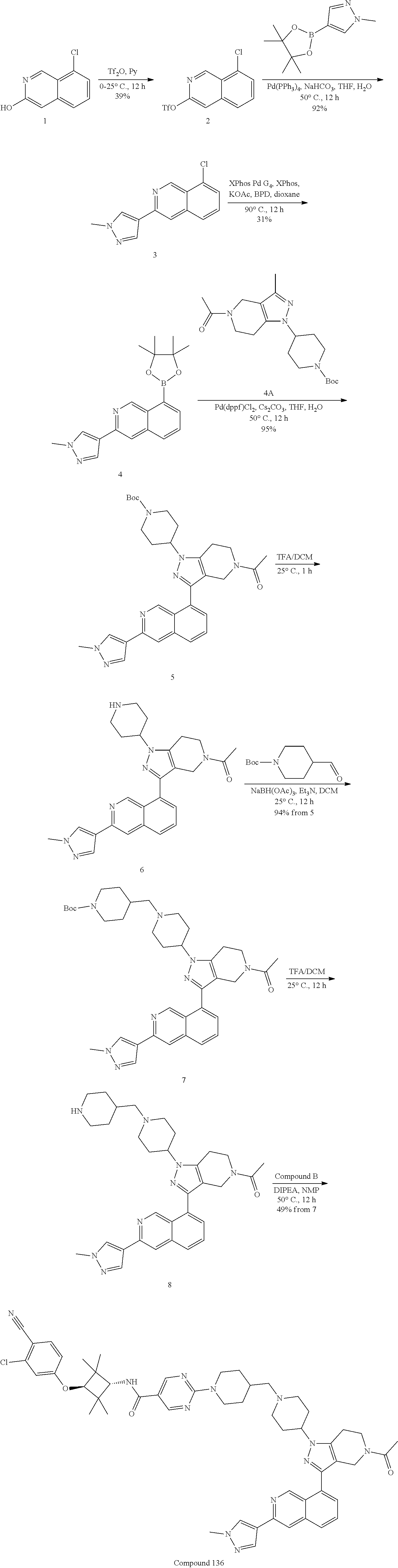

Synthesis of 1 was described in the report of Dalton Transactions, 2008, vol. 8, #4, p. 491-498.

To a solution of 8-chloroisoquinolin-3-ol (3.50 g, 19.5 mmol, 1.0 eq) in Py (90 mL) was added Tf₂O (7.15 g, 25.3 mmol, 1.3 eq) at 0° C. The mixture was stirred at 25° C. for 12 h under N₂. The mixture was poured into water (50 mL). The aqueous phase was extracted with ethyl acetate 150 mL (50 mL×3), dried with anhydrous sodium sulfate, filtered and concentrated under vacuum. The residue was purified by column chromatography (SiO₂, Petroleum ether/Ethyl acetate=5/1 to 0/1). Compound (8-chloro-3-isoquinolyl) trifluoromethanesulfonate (2.50 g, 7.62 mmol, 39% yield) was obtained as a white solid.

LC-MS: MS (ESI⁺): t_{R}=0.660 min, m/z=311.9 [M+H⁺]

To a solution of (8-chloro-3-isoquinolyl) trifluoromethanesulfonate (2.50 g, 8.02 mmol, 1.0 eq) and 1-methyl-4-(4,4,5,5-tetramethyl-1,3,2-dioxaborolan-2-yl)pyrazole (2.00 g, 9.63 mmol, 1.2 eq) in THF (50 mL) and H₂O (10 mL) was added NaHCO₃ (2.02 g, 24.1 mmol, 3.0 eq) and Pd(PPh₃)₄ (927 mg, 802 μmol, 0.1 eq). The mixture was stirred at 50° C. for 12 h under N₂. The mixture was poured into water (100 mL). The aqueous phase was extracted with ethyl acetate (80 mL×3), dried with anhydrous sodium sulfate, filtered and concentrated under vacuum. The residue was purified by prep-HPLC (column: Phenomenex luna C18 (250×70 mm, 10 um); mobile phase: [water(TFA)-ACN]; gradient: 23%-53% B over 20 min). Compound 8-chloro-3-(1-methylpyrazol-4-yl)isoquinoline (2.03 g, 7.39 mmol, 92% yield) was obtained as a yellow solid.

LC-MS: MS (ESI⁺): t_{R}=0.533 min, m/z=244.0 [M+H⁺]

To a solution of 8-chloro-3-(1-methylpyrazol-4-yl)isoquinoline (1.00 g, 4.10 mmol, 1.0 eq) and 4,4,5,5-tetramethyl-2-(4,4,5,5-tetramethyl-1,3,2-dioxaborolan-2-yl)-1,3,2-dioxaborolane (1.25 g, 4.92 mmol, 1.2 eq) in dioxane (20 mL) was added KOAc (1.21 g, 12.3 mmol, 3.0 eq) and Xphos Pd G4 (353 mg, 410 μmol, 0.1 eq) and XPhos (196 mg, 410 μmol, 0.1 eq). The mixture was stirred at 90° C. for 12 h under N₂. The mixture was poured into water (60 mL). The aqueous phase was extracted with ethyl acetate (60 mL×3), dried with anhydrous sodium sulfate, filtered and concentrated under vacuum. The residue was purified by column chromatography (SiO₂, Petroleum ether/Ethyl acetate=10/1 to 1/1). Compound 3-(1-methylpyrazol-4-yl)-8-(4,4,5,5-tetramethyl-1,3,2-dioxaborolan-2-yl)isoquinoline (450 mg, 1.28 mmol, 31% yield) was obtained as a yellow solid.

LC-MS: MS (ESI⁺): t_{R}=0.541 min, m/z=336.2 [M+H⁺]

To a solution of 3-(1-methylpyrazol-4-yl)-8-(4,4,5,5-tetramethyl-1,3,2-dioxaborolan-2-yl)isoquinoline (450 mg, 1.34 mmol, 1.2 eq) and tert-butyl 4-(5-acetyl-3-iodo-6,7-dihydro-4H-pyrazolo[4,3-c]pyridin-1-yl)piperidine-1-carboxylate (531 mg, 1.12 mmol, 1.0 eq) in THF (20 mL) and H₂O (4 mL) was added Cs₂CO₃ (1.09 g, 3.36 mmol, 3.0 eq) and Pd(dppf)Cl₂ (164 mg, 224 μmol, 0.2 eq). The mixture was stirred at 50° C. for 12 h under N₂. The mixture was poured into ice-water (50 mL). The aqueous phase was extracted with dichloromethane (50 mL×3). The combined organic phase was washed with brine (30 mL×2), dried with anhydrous sodium sulfate, filtered and concentrated under vacuum. The residue was purified by prep-HPLC (column: Phenomenex luna C18 150×40 mm×15 um; mobile phase: [water(FA)-ACN]; gradient: 40%-70% B over 15 min). Compound tert-butyl 4-[5-acetyl-3-[3-(1-methylpyrazol-4-yl)-8-isoquinolyl]-6,7-dihydro-4H-pyrazolo[4,3-c]pyridin-1-yl]piperidine-1-carboxylate (600 mg, 1.06 mmol, 95% yield) was obtained as a yellow oil.

LC-MS: MS (ESI⁺): t_{R}=0.538 min, m/z=556.5 [M+H⁺]

To a solution of tert-butyl 4-[5-acetyl-3-[3-(1-methylpyrazol-4-yl)-8-isoquinolyl]-6,7-dihydro-4H-pyrazolo[4,3-c]pyridin-1-yl]piperidine-1-carboxylate (300 mg, 540 μmol, 1.0 eq) in DCM (2 mL) was added TFA (768 mg, 6.73 mmol, 12.5 eq). The mixture was stirred at 25° C. for 1 h under N₂. The mixture was concentrated to give a residue. Compound 1-[3-[3-(1-methylpyrazol-4-yl)-8-isoquinolyl]-1-(4-piperidyl)-6,7-dihydro-4H-pyrazolo[4,3-c]pyridin-5-yl]ethanone (300 mg) as a yellow oil and directly used in the next step without further purification.

LC-MS: MS (ESI⁺): t_{R}=0.435 min, m/z=456.3 [M+H⁺]

1-[3-[3-(1-methylpyrazol-4-yl)-8-isoquinolyl]-1-(4-piperidyl)-6,7-dihydro-4H-pyrazolo[4,3-c]pyridin-5-yl]ethanone (100 mg) was added to the mixture of tert-butyl 4-formylpiperidine-1-carboxylate (56 mg, 263 μmol, 1.5 eq) and Et₃N (178 mg, 1.76 mmol, 10.0 eq) in DCM (3 mL) and stirred at 25° C. for 0.5 h. NaBH(OAc)₃ (186 mg, 878 μmol, 5.0 eq) was added to the above reaction mixture and stirred at 25° C. for 12 h under N₂. The mixture was poured into water (20 mL). The aqueous phase was extracted with ethyl acetate (20 mL×3), dried with anhydrous sodium sulfate, filtered and concentrated under vacuum. The residue was purified by prep-TLC (SiO₂, DCM:MeOH=15:1). Compound tert-butyl 4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]piperidine-1-carboxylate (120 mg, 165 μmol, 94% yield over two steps) was obtained as a yellow oil.

LC-MS: MS (ESI⁺): t_{R}=0.498 min, m/z=653.5 [M+H⁺]

To a solution of tert-butyl 4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]piperidine-1-carboxylate (120 mg, 165 μmol, 1.0 eq) in DCM (1 mL) was added TFA (768 mg, 6.73 mmol, 36.6 eq). The mixture was stirred at 25° C. for 1 h under N₂. The mixture was concentrated to give a residue. Compound 1-[3-[3-(1-methylpyrazol-4-yl)-8-isoquinolyl]-1-[1-(4-piperidylmethyl)-4-piperidyl]-6,7-dihydro-4H-pyrazolo[4,3-c]pyridin-5-yl]ethanone (120 mg) as a yellow oil and directly used in the next step without further purification.

LC-MS: MS (ESI⁺): t_{R}=0.440 min, m/z=553.4 [M+H⁺]

To a solution of 1-[3-[3-(1-methylpyrazol-4-yl)-8-isoquinolyl]-1-[1-(4-piperidylmethyl)-4-piperidyl]-6,7-dihydro-4H-pyrazolo[4,3-c]pyridin-5-yl]ethanone (120 mg) and 2-chloro-N-[3-(3-chloro-4-cyano-phenoxy)-2,2,4,4-tetramethyl-cyclobutyl]pyrimidine-5-carboxamide (91 mg, 216 μmol, 1.2 eq) in NMP (2 mL) was added DIPEA (70 mg, 540 μmol, 3.0 eq). The mixture was stirred at 50° C. for 12 h under N₂. The mixture was poured into water (20 mL). The aqueous phase was extracted with ethyl acetate (20 mL×3), dried with anhydrous sodium sulfate, filtered and concentrated under vacuum. The residue was purified by prep-HPLC (column: Phenomenex luna C18 150×25 mm×10 um; mobile phase: [water(FA)-ACN]; gradient: 32%-62% B over 10 min) to give 2-[4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-1-piperidyl]-N-[3-(3-chloro-4-cyano-phenoxy)-2,2,4,4-tetramethyl-cyclobutyl]pyrimidine-5-carboxamide (86.05 mg, 88.9 μmol, 49% yield over two steps) was obtained as an off-white solid.

¹H NMR (400 MHz, CHLOROFORM-d) δ=9.78-9.60 (m, 1H), 8.69 (s, 2H), 8.05 (m, 2H), 7.87-7.76 (m, 2H), 7.75-7.65 (m, 1H), 7.61-7.55 (m, 1H), 7.54-7.42 (m, 1H), 6.97 (d, J=2.4 Hz, 1H), 6.86-6.77 (m, 1H), 6.00-5.90 (m, 1H), 4.93-4.78 (m, 2H), 4.67 (s, 1H), 4.49 (s, 1H), 4.13 (d, J=8.0 Hz, 1H), 4.07-3.94 (m, 5H), 3.84 (m, 1H), 3.64-3.04 (m, 2H), 3.01-2.80 (m, 4H), 2.57-2.27 (m, 4H), 2.22 (s, 2H), 2.18-2.14 (m, 1H), 2.12-2.04 (m, 2H), 2.02-1.75 (m, 8H), 1.25 (s, 6H), 1.22 (s, 6H)

LC-MS: MS (ESI⁺): t_{R}=1.696 min, m/z=935.6 [M+H⁺]

The compounds below were prepared in a similar manner as described in Example 21.

| | | | LC-MS |
|---|---|---|---|
| Compound | Name | ¹H NMR (CDCl₃) | [M + H⁺] |
| 142 | 2-[4-[[4-[5-acetyl-3-[3-(1- | δ = 9.78-9.60 (m, 1H), 8.69 (s, 2H), | 935.6 |
| | methylpyrazol-4-yl)-8- | 8.05 (m, 2H), 7.87-7.76 (m, 2H), 7.75-7.65 | |
| | isoquinolyl]-6,7-dihydro-4H- | (m, 1H), 7.61-7.55 (m, 1H), 7.54-7.42 | |
| | pyrazolo[4,3-c]pyridin-1-yl]-1- | (m, 1H), 6.97 (d, J = 2.4 Hz, 1H), | |
| | piperidyl]methyl]-1-piperidyl]- | 6.86-6.77 (m, 1H), 6.00-5.90 (m, 1H), | |
| | N-[3-(3-chloro-4-cyano- | 4.93-4.78 (m, 2H), 4.67 (s, 1H), 4.49 (s, | |
| | phenoxy)-2,2,4,4-tetramethyl- | 1H), 4.13 (d, J = 8.0 Hz, 1H), 4.07-3.94 | |
| | cyclobutyl]pyrimidine-5- | (m, 5H), 3.84 (m, 1H), 3.64-3.04 (m, | |
| | carboxamide | 2H), 3.01-2.80 (m, 4H), 2.57-2.27 (m, | |
| | | 4H), 2.22 (s, 2H), 2.18-2.14 (m, 1H), | |
| | | 2.12-2.04 (m, 2H), 2.02-1.75 (m, 8H), | |
| | | 1.25 (s, 6H), 1.22 (s, 6H) | |
| 141 | 2-[4-[2-[4-[5-acetyl-3-[3-(1- | δ = 9.73-9.65 (m, 1H), 8.68 (s, 2H), | 949.7 |
| | methylpyrazol-4-yl)-8- | 8.51-8.38 (m, 1H), 8.09-7.99 (m, 2H), | |
| | isoquinolyl]-6,7-dihydro-4H- | 7.85-7.76 (m, 2H), 7.74-7.66 (m, 1H), | |
| | pyrazolo[4,3-c]pyridin-1-yl]-1- | 7.57 (d, J = 8.8 Hz, 1H), 7.52-7.42 (m, | |
| | piperidyl]ethyl]-1-piperidyl]-N- | 1H), 6.96 (d, J = 2.4 Hz, 1H), 6.85-6.77 | |
| | [3-(3-chloro-4-cyano-phenoxy)- | (m, 1H), 6.05-5.90 (m, 1H), 4.91-4.77 | |
| | 2,2,4,4-tetramethyl- | (m, 2H), 4.69-4.61 (m, 1H), 4.51-4.43 | |
| | cyclobutyl]pyrimidine-5- | (m, 1H), 4.32-4.10 (m, 2H), 4.06-4.03 | |
| | carboxamide | (m, 1H), 4.03-3.97 (m, 4H), 3.88-3.79 | |
| | | (m, 1H), 3.38-3.26 (m, 2H), 3.01-2.81 | |
| | | (m, 5H), 2.75-2.54 (m, 3H), 2.50-2.35 | |
| | | (m, 3H), 2.26-2.15 (m, 3H), 2.07 (s, | |
| | | 2H), 1.87-1.75 (m, 2H), 1.62-1.49 (m, | |
| | | 2H), 1.25 (s, 6H), 1.21 (s, 6H), 1.18-1.15 | |
| | | (m, 1H) | |

### Example 22: Synthesis of 2-[4-[[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]methyl]-1-piperidyl]-N-[3-(3-chloro-4-cyano-phenoxy)-2,2,4,4-tetramethyl-cyclobutyl]pyrimidine-5-carboxamidine (Compound 53)

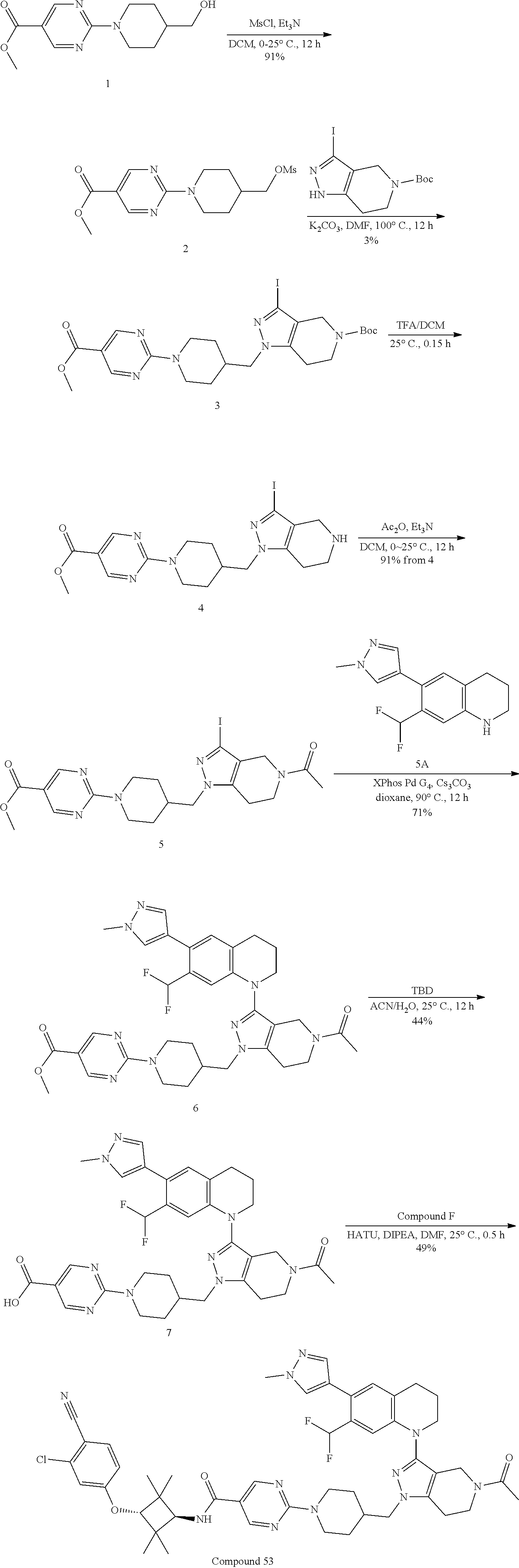

Synthesis of 1 was reported in WO2006/10750 A1.

Synthesis of 5A was reported in WO2016/86200 A1.

Synthesis of 7A was reported in Journal of Medicinal Chemistry, 2023, vol. 66, #7, p. 4784-4801.

To a solution of methyl 2-[4-(hydroxymethyl)-1-piperidyl]pyrimidine-5-carboxylate (4 g, 15.92 mmol, 1.0 eq) and Et₃N (8.05 g, 79.59 mmol, 11.08 mL, 5.0 eq) in DCM (50 mL) was added MsCl (3.29 g, 28.72 mmol, 2.22 mL, 1.8 eq) at 0° C. The reaction mixture was stirred at 25° C. for 12 h under N₂. The reaction mixture was quenched by addition water (30 mL) at 0° C., and then diluted with water 30 mL and extracted with DCM (30 mL×3). The combined organic layers were washed with brine (50 mL×3), dried over anhydrous sodium sulfate, filtered and concentrated under reduced pressure to give a residue. The residue was purified by column chromatography (silica, petroleum ether: ethyl acetate=10:1 to 1:1). The desired compound methyl 2-[4-(methylsulfonyloxymethyl)-1-piperidyl]pyrimidine-5-carboxylate (4.8 g, 14.57 mmol, 91% yield) was obtained as a white solid.

¹H NMR (400 MHz, CDCl₃): δ=8.82 (s, 2H), 4.97 (d, J=13.6 Hz, 2H), 4.10 (d, J=6.4 Hz, 2H), 3.87 (s, 3H), 3.02 (s, 3H), 2.96 (dt, J=2.4, 12.8 Hz, 2H), 2.20-2.05 (m, 1H), 1.90 (d, J=12.4 Hz, 2H), 1.31 (dq, J=4.0, 12.4 Hz, 2H).

LC-MS: MS (ESI⁺): t_{R}=0.476 min, m/z=330.1 [M+H⁺]

To a solution of tert-butyl 3-iodo-1,4,6,7-tetrahydropyrazolo[4,3-c]pyridine-5-carboxylate (4.8 g, 13.75 mmol, 1.0 eq) and methyl 2-[4-(methylsulfonyloxymethyl)-1-piperidyl]pyrimidine-5-carboxylate (4.80 g, 14.57 mmol, 1.06 eq) in DMF (50 mL) was added K₂CO₃ (5.70 g, 41.24 mmol, 3.0 eq). The reaction mixture was stirred at 100° C. for 12 h. The reaction mixture was quenched by addition water (30 mL) at 0° C., and then diluted with water (30 mL) and extracted with ethyl acetate (100 mL×3). The combined organic layers were washed with brine (50 mL×3), dried over anhydrous sodium sulfate, filtered and concentrated under reduced pressure to give a residue. The residue was purified by prep-HPLC (FA condition: column: Phenomenex luna C18 150*40 mm*15 um; mobile phase: [water (FA)-ACN]; gradient: 50%-80% B over 15 min). The desired compound tert-butyl 3-iodo-1-[[1-(5-methoxycarbonylpyrimidin-2-yl)-4-piperidyl]methyl]-6,7-dihydro-4H-pyrazolo[4,3-c]pyridine-5-carboxylate (310 mg, 532.25 μmol, 3% yield) was obtained as a white solid.

¹H NMR (400 MHz, CDCl₃): δ=8.82 (s, 2H), 4.93 (br d, J=13.6 Hz, 2H), 4.23 (br s, 2H), 3.92-3.84 (m, 5H), 3.70 (br s, 2H), 2.98-2.83 (m, 2H), 2.62 (br s, 2H), 2.37-2.22 (m, 1H), 1.70 (br d, J=11.2 Hz, 2H), 1.50 (s, 9H), 1.30-1.16 (m, 2H)

LC-MS: MS (ESI⁺): t_{R}=0.625 min, m/z=583.1 [M+H⁺]

To a solution of tert-butyl 3-iodo-1-[[1-(5-methoxycarbonylpyrimidin-2-yl)-4-piperidyl]methyl]-6,7-dihydro-4H-pyrazolo[4,3-c]pyridine-5-carboxylate (310 mg, 532.25 μmol, 1.0 eq) in DCM (10 mL) was added TFA (303 mg, 2.66 mmol, 197.68 μL, 5.0 eq). The reaction mixture was stirred at 25° C. for 0.15 h. The reaction mixture was concentrated under reduced pressure to give a residue. Compound methyl 2-[4-[(3-iodo-4,5,6,7-tetrahydropyrazolo[4,3-c]pyridin-1-yl)methyl]-1-piperidyl]pyrimidine-5-carboxylate (260 mg) was obtained as yellow oil and directly used in the next step without further purification.

LC-MS: MS (ESI⁺): t_{R}=0.422 min, m/z=483.2 [M+H⁺]

To a solution of methyl 2-[4-[(3-iodo-4,5,6,7-tetrahydropyrazolo[4,3-c]pyridin-1-yl)methyl]-1-piperidyl]pyrimidine-5-carboxylate (260 mg, 539.06 μmol, 1.0 eq) and Et₃N (164 mg, 1.62 mmol, 225.09 μL, 3.0 eq) in DCM (10 mL) was added Ac₂O (83 mg, 808.60 μmol, 75.94 μL, 1.5 eq) at 0° C. The reaction mixture was stirred at 25° C. for 12 h. The reaction mixture was quenched by addition water (20 mL) at 0° C., and then diluted with water 30 mL and extracted with DCM (40 mL×3). The combined organic layers were washed with brine (50 mL×3), dried over anhydrous sodium sulfate, filtered and concentrated under reduced pressure to give a residue. The residue was purified by prep-HPLC (FA condition: column: Welch Xtimate C18 150*25 mm*5 um; mobile phase: [water (FA)-ACN]; gradient: 30%-60% B over 10 min). The desired compound methyl 2-[4-[(5-acetyl-3-iodo-6,7-dihydro-4H-pyrazolo[4,3-c]pyridin-1-yl)methyl]-1-piperidyl]pyrimidine-5-carboxylate (260 mg, 492.77 μmol, 91% yield) was obtained as a white solid.

¹H NMR (400 MHz, CDCl3): δ=8.82 (s, 2H), 4.93 (br d, J=13.6 Hz, 2H), 4.23 (br s, 2H), 3.92-3.84 (m, 5H), 3.70 (br s, 2H), 2.98-2.83 (m, 2H), 2.62 (br s, 2H), 2.37-2.22 (m, 1H), 1.70 (br d, J=11.2 Hz, 2H), 1.50 (s, 9H), 1.30-1.16 (m, 2H)

LC-MS: MS (ESI⁺): t_{R}=0.503 min, m/z=525.2 [M+H⁺]

To a solution of methyl 2-[4-[(5-acetyl-3-iodo-6,7-dihydro-4H-pyrazolo[4,3-c]pyridin-1-yl)methyl]-1-piperidyl]pyrimidine-5-carboxylate (500 mg, 953.55 μmol, 1.0 eq) and 7-(difluoromethyl)-6-(1-methylpyrazol-4-yl)-1,2,3,4-tetrahydroquinoline (377 mg, 1.43 mmol, 1.5 eq) in dioxane (10 mL) was added Cs₂CO₃ (932 mg, 2.86 mmol, 3.0 eq) and Xphos Pd G₄ (82 mg, 95.36 μmol, 0.1 eq). The reaction mixture was stirred at 90° C. for 12 h under N₂. The reaction mixture was concentrated under reduced pressure to give a residue. The residue was purified by column chromatography (silica, petroleum ether/ethyl acetate=1:1 to DCM:MeOH=10:1). The desired compound methyl 2-[4-[[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]methyl]-1-piperidyl]pyrimidine-5-carboxylate (450 mg, 682.10 μmol, 71% yield) was obtained as a light yellow solid.

LC-MS: MS (ESI⁺): t_{R}=0.536 min, m/z=660.4 [M+H⁺]

To a solution of methyl 2-[4-[[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]methyl]-1-piperidyl]pyrimidine-5-carboxylate (250 mg, 378.94 mol, 1.0 eq) in ACN (6 mL) and H₂O (1 mL) was added 3,4,6,7,8,9-hexahydro-2H-pyrimido[1,2-a]pyrimidine (158 mg, 1.14 mmol, 3.0 eq). The reaction mixture was stirred at 25° C. for 12 h. The reaction mixture was added 1 M HCl to pH=3-4, then concentrated under reduced pressure to give a residue. The residue was purified by prep-HPLC (HCl condition: column: Phenomenex luna C18 150*25 mm*10 um; mobile phase: [water (HCl)-CAN]; gradient: 30%-60% B over 10 min). The desired compound 2-[4-[[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]methyl]-1-piperidyl]pyrimidine-5-carboxylic acid (110 mg, 170.36 μmol, 44% yield) was obtained as a white solid.

LC-MS: MS (ESI⁺): t_{R}=0.489 min, m/z=646.3 [M+H⁺]

To a solution of 4-(3-amino-2,2,4,4-tetramethyl-cyclobutoxy)-2-chloro-benzonitrile (49 mg, 154.87 μmol, 1.0 eq) and DIPEA (100 mg, 774.35 μmol, 134.88 μL, 5.0 eq) in DMF (5 mL) was added 2-[4-[[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]methyl]-1-piperidyl]pyrimidine-5-carboxylic acid (100 mg, 154.87 μmol, 1.0 eq) and HATU (118 mg, 309.74 μmol, 2.0 eq). The reaction mixture was stirred at 25° C. for 0.5 h. The reaction mixture was quenched by addition water (10 mL) at 25° C., and then diluted with water (20 mL) and extracted with ethyl acetate (30 mL×3). The combined organic layers were washed with brine (30 mL×3), dried over anhydrous sodium sulfate, filtered and concentrated under reduced pressure to give a residue. The residue was purified by prep-HPLC (FA condition:column: Welch Xtimate C18 150*25 mm*5 um; mobile phase: [water (FA)-ACN]; gradient: 58%-88% B over 10 min). The desired compound 2-[4-[[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]methyl]-1-piperidyl]-N-[3-(3-chloro-4-cyano-phenoxy)-2,2,4,4-tetramethyl-cyclobutyl]pyrimidine-5-carboxamide (69.02 mg, 75.97 μmol, 49% yield) was obtained as a white solid.

1HNMR (400 MHz, CDCl₃): δ=8.70 (s, 2H), 7.64-7.50 (m, 2H), 7.41 (d, J=6.4 Hz, 1H), 7.10-6.94 (m, 2H), 6.88 (d, J=6.8 Hz, 1H), 6.81 (dd, J=2.4, 8.8 Hz, 1H), 6.72-6.35 (m, 1H), 5.94 (br d, J=8.0 Hz, 1H), 4.92 (d, J=13.2 Hz, 2H), 4.37-4.10 (m, 3H), 4.05 (s, 1H), 3.96 (d, J=2.4 Hz, 3H), 3.91 (br t, J=5.6 Hz, 1H), 3.86 (d, J=7.2 Hz, 2H), 3.80-3.63 (m, 3H), 3.01-2.84 (m, 4H), 2.80-2.66 (m, 2H), 2.37-2.25 (m, 1H), 2.18 (s, 1H), 2.15-12.00 (m, 4H), 1.74 (d, J=8.8 Hz, 2H), 1.24 (d, J=13.2 Hz, 14H)

LC-MS: MS (ES⁺): t_{R}=2.687 min, m/z=453.9 [M/2]

The compounds below were prepared in a similar manner as described in Example 22.

| | | | LC-MS |
|---|---|---|---|
| Compound | Name | ¹H NMR (CDCl₃) | [M + H⁺] |
| 9 | 2-[4-[3-[5-acetyl-3-[7- | (DMSO-d₆) δ = 8.76 (s, 2H), 7.89 (d, J = | 950.7 |
| | (difluoromethyl)-6-(1- | 8.8 Hz, 1H), 7.80-7.65 (m, 2H), 7.50 (s, | |
| | methylpyrazol-4-yl)-3,4- | 1H), 7.21 (d, J = 2.4 Hz, 1H), 7.10 (br s, | |
| | dihydro-2H-quinolin-1-yl]-6,7- | 1H), 7.04-6.98 (m, 1H), 6.96-6.60 (m, | |
| | dihydro-4H-pyrazolo[4,3-c] | 2H), 4.32-4.19 (m, 3H), 4.17-4.09 (m, | |
| | pyridin-1-yl] propoxy]-1- | 2H), 4.07-3.99 (m, 3H), 3.86 (s, 3H), | |
| | piperidyl]-N-[3-(3-chloro-4- | 3.76-3.64 (m, 2H), 3.61-3.51 (m, 3H), | |
| | cyano-phenoxy)-2,2,4,4- | 3.49-3.40 (m, 4H), 2.83 (br d, J = 5.6 | |
| | tetramethyl-cyclobutyl] | Hz, 3H), 2.76-2.64 (m, 1H), 2.06 (s, | |
| | pyrimidine-5-carboxamide | 2H), 2.03-1.92 (m, 5H), 1.91-1.82 (m, | |
| | | 2H), 1.46-1.35 (m, 2H), 1.21 (s, 6H), | |
| | | 1.11 (s, 6H) | |
| 43 | 2-[3-[5-acetyl-3-[7- | (DMSO-d₆): δ = 8.96 (s, 2 H) 8.04 d, | 867.6 |
| | (difluoromethyl)-6-(1- | J = 9.2 Hz, 1 H) 7.90 (d, J = 8.8 Hz, 1 H) | |
| | methylpyrazol-4-yl)-3,4- | 7.75 (s, 1 H) 7.49 (s, 1 H) 7.22 (d, J = 2.4 | |
| | dihydro-2H-quinolin-1-yl]-6,7- | Hz, 1 H) 7.10 (s, 1 H) 7.01 (m, 1 H) 6.61-6.94 | |
| | dihydro-4H-pyrazolo[4,3- | (m, 2 H) 4.32-4.41 (m, 2 H) 4.30 | |
| | c]pyridin-1-yl]propoxy]-N-[3- | (s, 1 H) 4.10-4.17 (m, 3 H) 4.05-4.09 | |
| | (3-chloro-4-cyano-phenoxy)- | (m, 2 H) 3.86 (s, 3 H) 3.62-3.72 (m, | |
| | 2,2,4,4-tetramethyl- | 3 H) 3.53-3.57 (m, 2 H) 2.82 (m, 4 H) | |
| | cyclobutyl]pyrimidine-5- | 2.22-2.27 (m, 2 H) 2.04 (s, 2 H) 1.94 (s, | |
| | carboxamide | 2 H) 1.22 (s, 6 H) 1.12 (s, 6 H). | |
| 44 | 2-[3-[5-acetyl-3-[7- | (DMSO-d6) δ = 8.73 (s, 2H), 8.18 (s, | 880.6 |
| | (difluoromethyl)-6-(1- | 1H), 7.89 (d, J = 8.8 Hz, 1H), 7.77-7.67 | |
| | methylpyrazol-4-yl)-3,4- | (m, 2H), 7.49 (s, 1H), 7.21 (d, J = 2.4 | |
| | dihydro-2H-quinolin-1-yl]-6,7- | Hz, 1H), 7.10 (s, 1H), 7.04-6.96 (m, | |
| | dihydro-4H-pyrazolo[4,3- | 1H), 6.94-6.60 (m, 2H), 4.28 (s, 1H), | |
| | c]pyridin-1-yl]propyl-methyl- | 4.16-4.08 (m, 2H), 4.05-3.97 (m, 3H), | |
| | amino]-N-[3-(3-chloro-4- | 3.86 (s, 3H), 3.73-3.64 (m, 4H), 3.61-3.56 | |
| | cyano-phenoxy)-2,2,4,4- | (m, 2H), 3.17-3.13 (m, 3H), 2.86-2.81 | |
| | tetramethyl- | (m, 2H), 2.80-2.76 (m, 1H), 2.69-2.63 | |
| | cyclobutyl]pyrimidine-5- | (m, 1H), 2.13-2.04 (m, 4H), 2.01-1.94 | |
| | carboxamide | (m, 3H), 1.20 (s, 6H), 1.10 (s, 6H) | |
| 45 | 2-[2-[5-acetyl-3-[7- | (DMSO-d₆) δ = 8.91-8.60 (m, 2H), 7.89 | 866.6 |
| | (difluoromethyl)-6-(1- | (br d, J = 8.8 Hz, 1H), 7.79-7.67 (m, | |
| | methylpyrazol-4-yl)-3,4- | 2H), 7.49 (s, 1H), 7.21 (br s, 1H), 7.15-7.06 | |
| | dihydro-2H-quinolin-1-yl]-6,7- | (m, 1H), 7.00 (br d, J = 8.8 Hz, 1H), | |
| | dihydro-4H-pyrazolo[4,3- | 6.95-6.61 (m, 2H), 4.29 (s, 1H), 4.26-4.19 | |
| | c]pyridin-1-yl]ethyl-methyl- | (m, 2H), 4.11-3.95 (m, 5H), 3.86 | |
| | amino]-N-[3-(3-chloro-4- | (s, 3H), 3.69-3.57 (m, 2H), 3.56-3.47 | |
| | cyano-phenoxy)-2,2,4,4- | (m, 2H), 3.28-3.20 (m, 1H), 3.05-2.92 | |
| | tetramethyl- | (m, 3H), 2.87-2.77 (m, 2H), 2.64 (br s, | |
| | cyclobutyl]pyrimidine-5- | 1H), 2.07 (s, 2H), 1.99-1.87 (m, 3H), | |
| | carboxamide | 1.22 (s, 6H), 1.11 (s, 6H) | |
| 46 | 2-[2-[5-acetyl-3-[7- | (DMSO-d₆) δ = 8.94 (s, 2H), 7.84 (d, J = | 853.6 |
| | (difluoromethyl)-6-(1- | 8.8 Hz, 1H), 7.78 (br d, J = 8.8 Hz, 1H), | |
| | methylpyrazol-4-yl)-3,4- | 7.69 (s, 1H), 7.46 (s, 1H), 7.18 (d, J = 2.4 | |
| | dihydro-2H-quinolin-1-yl]-6,7- | Hz, 1H), 7.09 (s, 1H), 7.04-6.99 (m, | |
| | dihydro-4H-pyrazolo[4,3- | 1H), 6.88-6.56 (m, 2H), 4.79 (t, J = 5.6 | |
| | c]pyridin-1-yl]ethoxy]-N-[3-(3- | Hz, 2H), 4.40 (t, J = 5.2 Hz, 2H), 4.30 (s, | |
| | chloro-4-cyano-phenoxy)- | 1H), 4.13-4.05 (m, 3H), 3.87 (s, 3H), | |
| | 2,2,4,4-tetramethyl- | 3.71 (br t, J = 5.2 Hz, 2H), 3.60-3.51 | |
| | cyclobutyl]pyrimidine-5- | (m, 2H), 2.84 (br t, J = 5.6 Hz, 4H), 2.10-1.91 | |
| | carboxamide | (m, 5H), 1.25 (s, 6H), 1.16 (s, 6H) | |
| 51 | 2-[4-[2-[5-acetyl-3-[7- | δ = 8.68 (d, J = 1.6 Hz, 2H), 7.61-7.49 | 468.9 |
| | (difluoromethyl)-6-(1- | (m, 2H), 7.40 (d, J = 6.4 Hz, 1H), 7.10-6.99 | [M/2] |
| | methylpyrazol-4-yl)-3,4- | (m, 1H), 6.96 (d, J = 2.0 Hz, 1H), | |
| | dihydro-2H-quinolin-1-yl]-6,7- | 6.87 (d, J = 3.2 Hz, 1H), 6.80 (dd, J = | |
| | dihydro-4H-pyrazolo[4,3- | 2.0, 8.8 Hz, 1H), 6.69-6.35 (m, 1H), | |
| | c]pyridin-1-yl]ethoxy]-1- | 6.17-5.94 (m, 1H), 4.29-4.02 (m, 8H), | |
| | piperidyl]-N-[3-(3-chloro-4- | 3.95 (d, J = 2.4 Hz, 3H), 3.90-3.81 (m, | |
| | cyano-phenoxy)-2,2,4,4- | 3H), 3.78-3.65 (m, 3H), 3.65-3.50 (m, | |
| | tetramethyl- | 3H), 2.95-2.75 (m, 4H), 2.15-2.00 (m, | |
| | cyclobutyl]pyrimidine-5- | 5H), 1.89-1.79 (m, 2H), 1.61-1.45 (m, | |
| | carboxamide | 2H), 1.23 (d, J = 14.4 Hz, 12H). | |
| 52 | 2-[4-[2-[5-acetyl-3-[7- | δ = 8.71 (d, J = 2.80 Hz, 2 H) 7.58 (d, | 921.6 |
| | (difluoromethyl)-6-(1- | J = 8.80 Hz, 1 H) 7.54 (d, J = 5.60 Hz, 1 H) | |
| | methylpyrazol-4-yl)-3,4- | 7.41 (d, J = 6.40 Hz, 1 H) 6.99-7.09 (m, | |
| | dihydro-2H-quinolin-1-yl]-6,7- | 1 H) 6.97 (d, J = 2.40 Hz, 1 H) 6.86 (s, | |
| | dihydro-4H-pyrazolo[4,3- | 1 H) 6.81 (dd, J = 8.80, 2.40 Hz, 1 H) 6.34-6.70 | |
| | c]pyridin-1-yl]ethyl]piperazin- | (m, 1 H) 5.89-6.04 (m, 1 H) 4.28 | |
| | 1-yl]-N-[3-(3-chloro-4-cyano- | (s, 1 H) 4.15 (br s, 2 H) 4.12 (s, 2 H) 4.05 | |
| | phenoxy)-2,2,4,4-tetramethyl- | (s, 1 H) 3.96 (s, 3 H) 3.91 (br d, J = 3.60 | |
| | cyclobutyl]pyrimidine-5- | Hz, 5 H) 3.65-3.82 (m, 3 H) 2.81-2.96 | |
| | carboxamide | (m, 5 H) 2.78 (br t, J = 5.20 Hz, 1 H) 2.50-2.62 | |
| | | (m, 4 H) 2.17 (s, 1 H) 1.98-2.15 | |
| | | (m, 4 H) 1.25 (s, 6 H) 1.22 (s, 6 H). | |

### Example 23: Synthesis of 6-[3-[[6-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro [3.3]heptan-2-yl]methyl]azetidin-1-yl]-N-[3-(3-chloro-4-cyano-phenoxy)-2,2,4,4-tetramethyl-cyclobutyl]pyridazine-3-carboxamide (Compound 212)

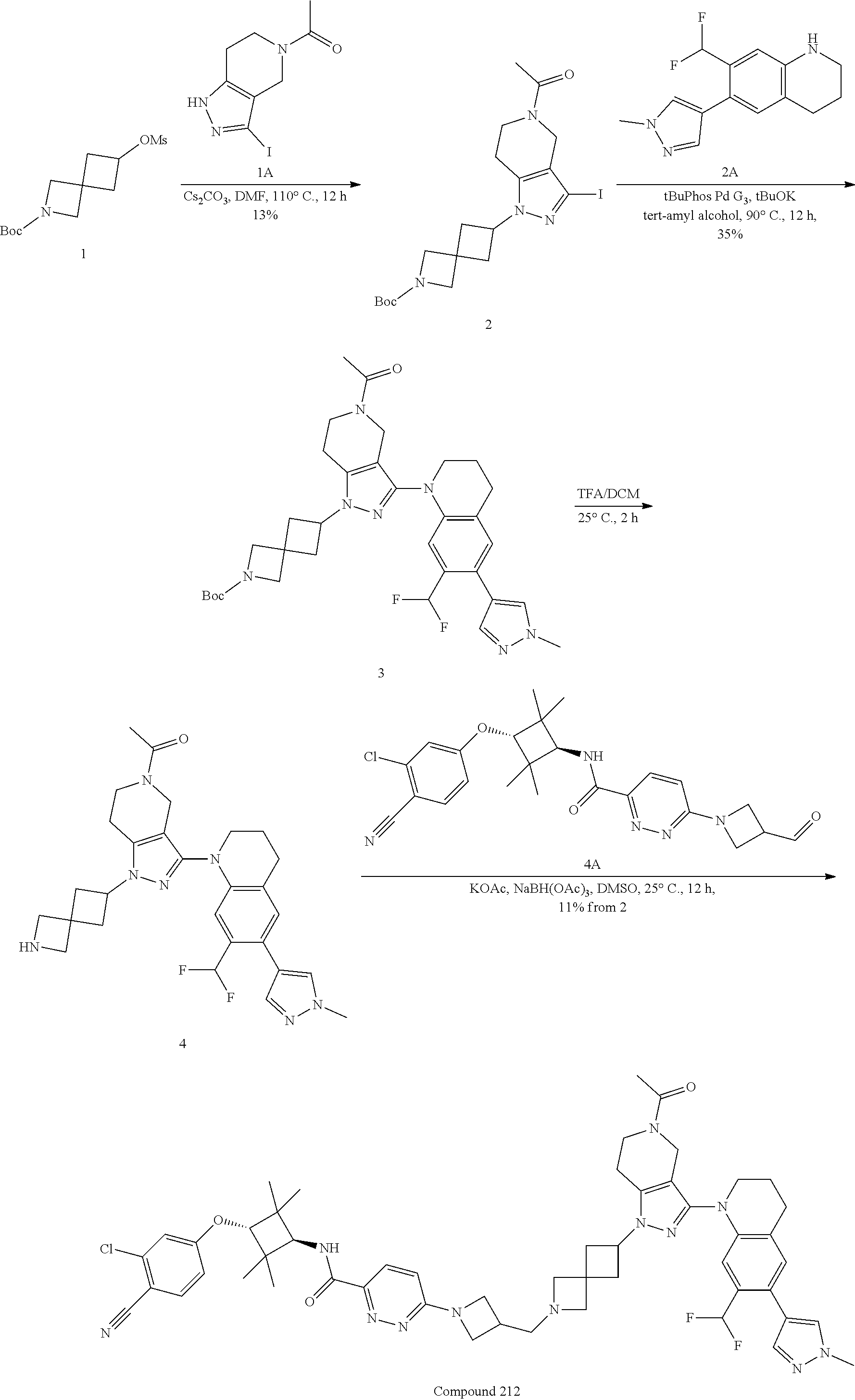

Synthesis of 1 was reported in WO2019/8025 A1.

Synthesis of 2A was reported in WO2016/86200 A1.

To a solution of tert-butyl 6-methylsulfonyloxy-2-azaspiro [3.3]heptane-2-carboxylate (3.40 g, 11.7 mmol, 2.0 eq), 1-(3-iodo-1,4,6,7-tetrahydropyrazolo[4,3-c]pyridin-5-yl) ethanone (1.68 g, 5.77 mmol, 1.0 eq), Cs₂CO₃ (5.64 g, 17.3 mmol, 3.0 eq) in DMF (10 mL). The mixture was stirred at 110° C. for 12 h under N₂. The reaction mixture was poured into ice water 50 ml at 0° C., and then extracted with DCM (20 mL×3). The combined organic layers were washed with brine 20 mL, dried over Na₂SO₄, filtered and concentrated under reduced pressure to give a residue. The crude product was purified by reversed-phase HPLC (column: Phenomenex luna C18 150×40 mm×15 um; mobile phase: [water (FA)-ACN]; gradient: 50%-80% B over 15 min). Compound tert-butyl 6-(5-acetyl-3-iodo-6,7-dihydro-4H-pyrazolo[4,3-c]pyridin-1-yl)-2-azaspiro [3.3]heptane-2-carboxylate (352 mg, 724 μmol, 13% yield) was obtained as a white solid.

LC-MS: MS (ESI⁺): t_{R}=0.572 min, m/z=487.2 [M+H⁺]

To a solution of tert-butyl 6-(5-acetyl-3-iodo-6,7-dihydro-4H-pyrazolo[4,3-c]pyridin-1-yl)-2-azaspiro[3.3]heptane-2-carboxylate (160 mg, 329 μmol, 1.0 eq), 7-(difluoromethyl)-6-(1-methylpyrazol-4-yl)-1,2,3,4-tetrahydroquinoline (106 mg, 40.3 μmol, 1.2 eq), [2-(2-aminophenyl)phenyl]-methylsulfonyloxy-palladium; ditert-butyl-[2-(2,4,6-triisopropylphenyl) phenyl]phosphane (63 mg, 79.0 μmol, 0.2 eq) in tert-amyl alcohol (10 mL). The mixture was added t-BuOK (1 M, 987 μL, 3.0 eq) and stirred at 90° C. for 12 h under N₂. The reaction mixture was poured into ice water 50 mL at 0° C., and then extracted with DCM (20 mL×3). The combined organic layers were washed with brine 20 mL, dried over Na₂SO₄, filtered and concentrated under reduced pressure to give a residue. The residue was purified by prep-TLC (SiO₂, DCM:MeOH=10:1). Compound tert-butyl 6-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro [3.3]heptane-2-carboxylate (71 mg, 114 μmol, 35% yield) was obtained as a white solid.

LC-MS: MS (ESI⁺): t_{R}=0.603 min, m/z=622.5 [M+H⁺]

To a solution of tert-butyl 6-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro [3.3]heptane-2-carboxylate (70 mg, 113 μmol, 1.0 eq) in DCM (0.5 mL) was added TFA (0.5 mL). The mixture was stirred at 25° C. for 2 h. The reaction mixture was concentrated under reduced pressure to give a residue. Compound 1-[1-(2-azaspiro [3,3]heptan-6-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (71 mg) was obtained as a white solid and directly used in the next step without further purification.

LC-MS: MS (ESI⁺): t_{R}=0.493 min, m/z=522.4 [M+H⁺]

To a solution of 1-[1-(2-azaspiro[3.3]heptan-6-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (71 mg), N-[3-(3-chloro-4-cyano-phenoxy)-2,2,4,4-tetramethyl-cyclobutyl]-6-(3-formylazetidin-1-yl)pyridazine-3-carboxamide (60 mg, 129 μmol, 1.2 eq), KOAc (33 mg, 335 μmol, 3.0 eq) in DMSO (1 mL). The mixture was stirred for 0.1 h and added NaBH(OAc)₃ (71 mg, 335 μmol, 3.0 eq). The mixture was stirred at 25° C. for 12 h under N₂. The reaction mixture was poured into ice water 50 mL at 0° C., and then extracted with DCM (20 mL×3). The combined organic layers were washed with brine 20 mL, dried over Na₂SO₄, filtered and concentrated under reduced pressure to give a residue. The crude product was purified by reversed-phase HPLC (column: Phenomenex luna C18 150×25 mm×10 um; mobile phase: [water (FA)-ACN]; gradient: 30%-60% B over 10 min). Compound 6-[3-[[6-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro [3.3]heptan-2-yl]methyl]azetidin-1-yl]-N-[3-(3-chloro-4-cyano-phenoxy)-2,2,4,4-tetramethyl-cyclobutyl]pyridazine-3-carboxamide (12 mg, 12.1 μmol, 11% yield over two steps) was obtained as a white solid.

¹H NMR (400 MHz, DMSO-d₆) δ=8.21 (br d, J=9.2 Hz, 1H), 7.90 (d, J=8.4 Hz, 1H), 7.83 (d, J=9.2 Hz, 1H), 7.75 (s, 1H), 7.50 (s, 1H), 7.25 (d, J=2.4 Hz, 1H), 7.11 (br s, 1H), 7.06-7.01 (m, 1H), 6.97-6.62 (m, 3H), 4.61 (brt, J=8.0 Hz, 1H), 4.45 (s, 1H), 4.22-4.07 (m, 4H), 3.99 (d, J=9.0 Hz, 1H), 3.86 (s, 3H), 3.82-3.74 (m, 2H), 3.74-3.65 (m, 2H), 3.63-3.56 (m, 2H), 3.33-3.33 (m, 1H), 3.20 (br s, 2H), 2.90-2.72 (m, 5H), 2.71-2.63 (m, 3H), 2.56 (br t, J=8.0 Hz, 4H), 2.11-2.03 (m, 2H), 2.02-1.92 (m, 3H), 1.21 (s, 6H), 1.13 (s, 6H).

LC-MS: MS (ESI⁺): t_{R}=2.255 min, m/z=973.6 [M+H⁺]

### Example 24: Synthesis of 2-[6-[[4-[8-(5-acetyl-1-tetrahydropyran-4-yl-6,7-dihydro-4H-pyrazolo[4,3-c]pyridin-3-yl)-3-isoquinolyl]pyrazol-1-yl]methyl]-2-azaspiro[3.3]heptan-2-yl]-N-[3-(3-chloro-4-cyano-phenoxy)-2,2,4,4-tetramethyl-cyclobutyl]pyrimidine-5-carboxamide (Compound 223)

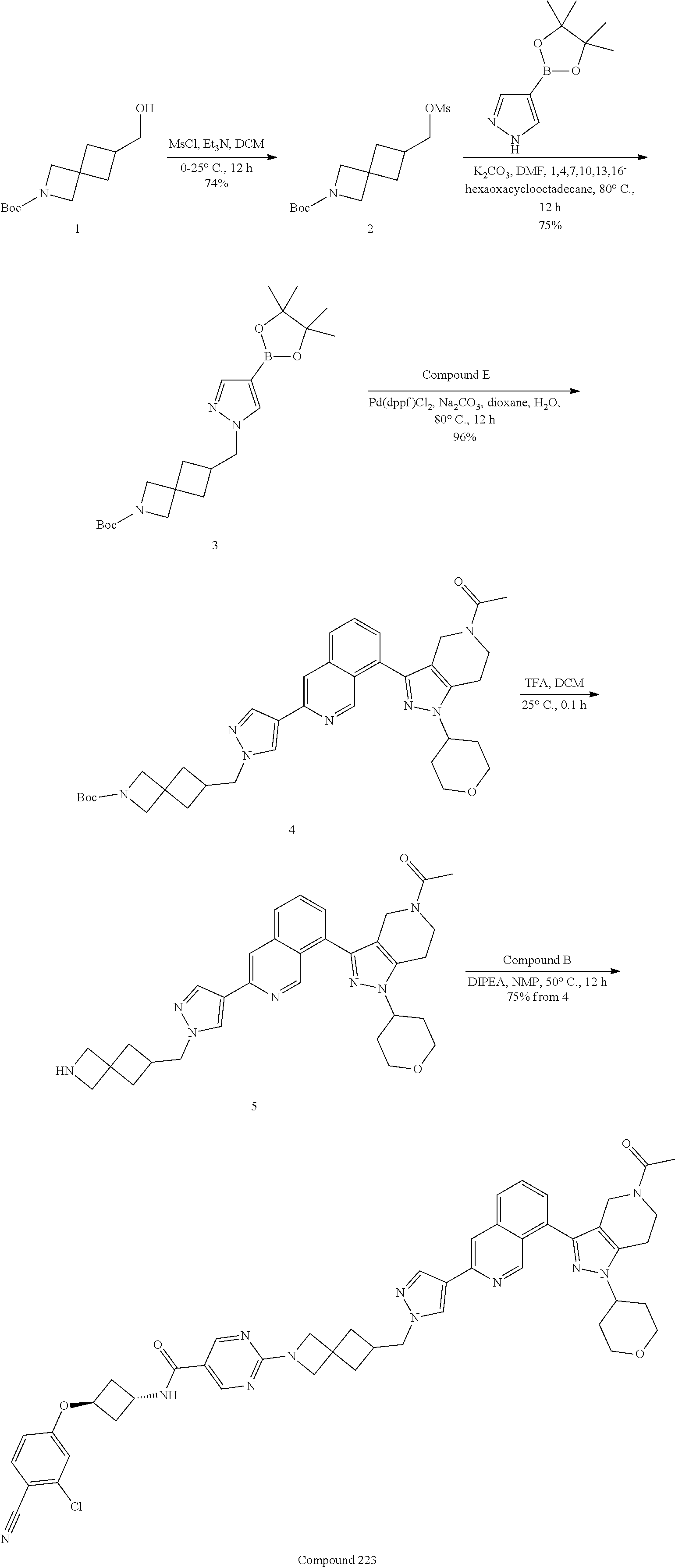

To a solution of tert-butyl 6-(hydroxymethyl)-2-azaspiro[3.3]heptane-2-carboxylate (2 g, 8.80 mmol, 1.0 eq) in DCM (20 mL) was added Et₃N (4.45 g, 43.99 mmol, 6.12 mL, 5.0 eq) and MsCl (1.72 g, 15.02 mmol, 1.16 mL, 1.71 eq) at 0° C. The mixture was stirred at 25° C. for 12 h. The mixture was diluted with ice water (80 mL) and extracted with ethyl acetate (100 mL×3). The combined organic layers were washed with brine (100 mL×3), dried over anhydrous sodium sulfate, filtered and concentrated in vacuum. The residue was purified by column chromatography (SiO₂, Petroleum ether/Ethyl acetate=1/0 to 5/1). Compound tert-butyl 6-(methylsulfonyloxymethyl)-2-azaspiro[3.3]heptane-2-carboxylate (2 g, 6.55 mmol, 74% yield) was obtained as a white solid.

¹H NMR (400 MHz, CDCl3) δ=4.16 (d, J=6.3 Hz, 1H), 3.99-3.91 (m, 2H), 3.90-3.82 (m, 2H), 3.58 (d, J=6.4 Hz, 1H), 3.03 (s, 2H), 2.75-2.46 (m, 1H), 2.39-2.24 (m, 2H), 2.09-1.91 (m, 2H), 1.60 (br d, J=5.1 Hz, 1H), 1.45 (s, 9H)

To a solution of 4-(4,4,5,5-tetramethyl-1,3,2-dioxaborolan-2-yl)-1H-pyrazole (1.27 g, 6.55 mmol, 1.0 eq), tert-butyl 6-(methylsulfonyloxymethyl)-2-azaspiro[3.3]heptane-2-carboxylate (2 g, 6.55 mmol, 1.0 eq) in DMF (20 mL) was added K₂CO₃ (2.72 g, 19.65 mmol, 3.0 eq) and 1,4,7,10,13,16-hexaoxacyclooctadecane (173 mg, 654.90 μmol, 0.1 eq). The mixture was stirred at 80° C. for 12 h. The mixture was diluted with water (60 mL) and extracted with ethyl acetate (80 mL×3). The combined organic layers were washed with brine (60 mL×3), dried over anhydrous sodium sulfate, filtered and concentrated in vacuum. The residue was purified by flash silica gel chromatography (ISCO®; 12 g SepaFlash® Silica Flash Column, Eluent of 0-20% Ethylacetate/Petroleum ethergradient @80 mL/min). Compound tert-butyl 6-[[4-(4,4,5,5-tetramethyl-1,3,2-dioxaborolan-2-yl)pyrazol-1-yl]methyl]-2-azaspiro[3.3]heptane-2-carboxylate (2 g, 4.96 mmol, 75% yield) was obtained as a white solid.

¹H NMR (400 MHz, CDCl3) δ=7.79 (s, 1H), 7.64 (s, 1H), 4.13-4.09 (m, 2H), 3.93 (s, 2H), 3.82 (s, 2H), 2.70 (td, J=7.7, 15.5 Hz, 1H), 2.31-2.26 (m, 2H), 2.02-1.93 (m, 2H), 1.44 (s, 9H), 1.33 (s, 11H)

LC-MS: MS (ESI⁺): t_{R}=0.581 min, m/z=348.0 [M−55]

A mixture of tert-butyl 6-[[4-(4,4,5,5-tetramethyl-1,3,2-dioxaborolan-2-yl)pyrazol-1-yl]methyl]-2-azaspiro[3.3]heptane-2-carboxylate (153.79 mg, 381.31 μmol, 2.0 eq), [8-(5-acetyl-1-tetrahydropyran-4-yl-6,7-dihydro-4H-pyrazolo[4,3-c]pyridin-3-yl)-3-isoquinolyl]trifluoromethanesulfonate (100 mg, 190.65 μmol, 1.0 eq), Pd(dppf)Cl₂ (13.95 mg, 19.07 μmol, 0.1 eq), Na₂CO₃ (101.04 mg, 953.27 μmol, 5 eq) and H₂O (0.5 mL) in dioxane (5 mL) was degassed and purged with N₂ for 3 times, and then the mixture was stirred at 80° C. for 12 h under N₂ atmosphere. The mixture was diluted with water (20 mL) and extracted with ethyl acetate (30 mL×3). The combined organic layers were washed with brine (20 mL×3), dried over anhydrous sodium sulfate, filtered and concentrated in vacuum. The residue was purified by prep-TLC (SiO₂, DCM:MeOH=15:1). Compound tert-butyl 6-[[4-[8-(5-acetyl-1-tetrahydropyran-4-yl-6,7-dihydro-4H-pyrazolo[4,3-c]pyridin-3-yl)-3-isoquinolyl]pyrazol-1-yl]methyl]-2-azaspiro[3.3]heptane-2-carboxylate (120 mg, 184.11 μmol, 96.57% yield) was obtained as a yellow solid.

LC-MS: MS (ESI⁺): t_{R}=0.480 min, m/z=652.5 [M+Na⁺]

To a solution of tert-butyl 6-[[4-[8-(5-acetyl-1-tetrahydropyran-4-yl-6,7-dihydro-4H-pyrazolo[4,3-c]pyridin-3-yl)-3-isoquinolyl]pyrazol-1-yl]methyl]-2-azaspiro[3.3]heptane-2-carboxylate (120 mg, 184.11 μmol, 1.0 eq) in DCM (2 mL) was added TFA (3.07 g, 26.92 mmol, 2 mL, 146.24 eq). The mixture was stirred at 25° C. for 0.1 h. The mixture was concentrated in vacuo. Compound 1-[3-[3-[1-(2-azaspiro[3.3]heptan-6-ylmethyl)pyrazol-4-yl]-8-isoquinolyl]-1-tetrahydropyran-4-yl-6,7-dihydro-4H-pyrazolo[4,3-c]pyridin-5-yl]ethanone (100 mg) was obtained as a yellow oil and directly used in the next step without further purification.

LC-MS: MS (ESI⁺): t_{R}=0.377 min, m/z=522.4 [M+H⁺]

To a solution of 1-[3-[3-[1-(2-azaspiro[3.3]heptan-6-ylmethyl)pyrazol-4-yl]-8-isoquinolyl]-1-tetrahydropyran-4-yl-6,7-dihydro-4H-pyrazolo[4,3-c]pyridin-5-yl]ethanone (100 mg, 181.26 μmol, 1.0 eq) in NMP (5 mL) was added DIPEA (117.14 mg, 906.32 μmol, 157.86 μL, 5.0 eq) and 2-chloro-N-[3-(3-chloro-4-cyano-phenoxy)-2,2,4,4-tetramethyl-cyclobutyl]pyrimidine-5-carboxamide (76.00 mg, 181.26 μmol, 1.0 eq). The mixture was stirred at 50° C. for 12 h. The mixture was diluted with water (20 mL) and extracted with ethyl acetate (30 mL×3). The combined organic layers were washed with brine (20 mL×3), dried over anhydrous sodium sulfate, filtered and concentrated in vacuum. The mixture was purified by prep-HPLC (column: Welch Xtimate C18 150*25 mm*5 um; mobile phase: [water(FA)-ACN]; gradient: 430%-73% B over 10 mi). Compound 2-[6-[[4-[8-(5-acetyl-1-tetrahydropyran-4-yl-6,7-dihydro-4H-pyrazolo[4,3-c]pyridin-3-yl)-3-isoquinolyl]pyrazol-1-yl]methyl]-2-azaspiro[3.3]heptan-2-yl]-[3-(3-chloro-4-cyano-phenoxy)-2,2,4,4-tetramethyl-cyclobutyl]pyrimidine-5-carboxamide (65.78 mg, 68.36 μmol, 37 yield over two steps) was obtained as an off-white solid.

¹H NMR (400 MHz, CDCl₃) δ=9.96-9.52 (m, 1H), 8.70 (s, 2H), 8.12-8.07 (m, 1H), 8.03-7.64 (m, 3H), 7.64-7.42 (m, 2H), 7.31-7.29 (m, 1H), 7.02-6.93 (m, 1H), 6.82 (dd, J=2.3, 8.7 Hz, 1H), 6.02-5.83 (m, 1H), 4.79-4.40 (m, 2H), 4.35-4.23 (m, 5H), 4.22-4.16 (m, 2H), 4.16-4.09 (m, 3H), 4.08-4.00 (m, 2H), 3.87 (brt, J=5.7 Hz, 1H), 3.68-3.51 (m, 2H), 2.99-2.84 (m, 3H), 2.54-2.40 (n, 4H), 2.2-2.17 (m, 3H), 2.10 (s, 1H), 2.06-1.94 (m, 2H), 1.30-1.20 (m, 13H)

LC-MS: MS (ESI⁺): t_{R}=2.675 min m/z=934.7 [M+H⁺]

The compounds below were prepared in a similar manner as described in Examples 23 and 24.

| | | | LC-MS |
|---|---|---|---|
| Compound | Name | ¹H NMR (CDCl₃) | [M + H⁺] |
| 209 | 6-[3-[[9-[5-acetyl-3-[7- | δ = 8.41-8.37 (m, 1H), 8.18 (d, J = 8.8 | 1029.7 |
| | (difluoromethyl)-6-(1- | Hz, 1H), 7.97 (d, J = 9.2 Hz, 1H), 7.62-7.50 | |
| | methylpyrazol-4-yl)-3,4-dihydro- | (m, 2H), 7.41 (d, J = 5.6 Hz, 1H), | |
| | 2H-quinolin-1-yl]-6, 7-dihydro- | 7.07-6.95 (m, 2H), 6.93-6.78 (m, 2H), | |
| | 4H-pyrazolo[4,3-c]pyridin-1-yl]- | 6.59 (d, J = 8.8 Hz, 2H), 4.42-4.32 (m, | |
| | 3-azaspiro[5.5]undecan-3- | 2H), 4.27 (s, 1H), 4.18 (d, J = 9.2 Hz, | |
| | yl]methyl]azetidin-1-yl]-N-[3-(3- | 1H), 4.13 (s, 1H), 4.07 (s, 1H), 4.00-3.88 | |
| | chloro-4-cyano-phenoxy)- | (m, 7H), 3.79-3.66 (m, 3H), 3.25-3.10 | |
| | 2,2,4,4-tetramethyl- | (m, 1H), 2.92-2.78 (m, 5H), 2.73 | |
| | cyclobutyl]pyridazine-3- | (s, 1H), 2.57 (d, J = 8.2 Hz, 4H), 2.22-2.15 | |
| | carboxamide | (m, 2H), 2.10-2.02 (m, 5H), 1.97-1.84 | |
| | | (m, 6H), 1.80 (s, 4H), 1.28 (s, 6H), | |
| | | 1.21 (s, 6H) | |
| 216 | 2-[6-[[6-[5-acetyl-3-[7- | (DMSO-d₆) δ = 8.72 (s, 2H), 7.90 (d, J = | 1013.8 |
| | (difluoromethyl)-6-(1- | 8.8 Hz, 1H), 7.75 (s, 1H), 7.73 (br d, J = | |
| | methylpyrazol-4-yl)-3,4-dihydro | 9.6 Hz, 1H), 7.50 (s, 1H), 7.21 (d, J = | |
| | 2H-quinolin-1-yl]-6,7-dihydro- | 2.4 Hz, 1H), 7.13-7.08 (m, 1H), 7.03-6.97 | |
| | 4H-pyrazolo[4,3-c]pyridin-1-yl]- | (m, 1H), 6.96-6.60 (m, 2H), 4.60 | |
| | 2-azaspiro[3.3]heptan-2- | (br t, J = 8.0 Hz, 1H), 4.28 (s, 1H), 4.17-4.08 | |
| | yl]methyl]-2- | (m, 4H), 4.03 (d, J = 9.2 Hz, 1H), | |
| | azaspiro[3.3]heptan-2-yl]-N-[3- | 3.99 (s, 2H), 3.86 (s, 3H), 3.74-3.64 | |
| | (3-chloro-4-cyano-phenoxy)- | (m, 2H), 3.62-3.56 (m, 2H), 3.26 (br s, | |
| | 2,2,4,4-tetramethyl- | 2H), 3.14 (br s, 2H), 2.87-2.81 (m, 2H), | |
| | cyclobutyl]pyrimidine-5- | 2.78 (br t, J = 3.2 Hz, 1H), 2.68-2.63 | |
| | carboxamide | (m, 1H), 2.61-2.52 (m, 4H), 2.40 (br d, | |
| | | J = 6.4 Hz, 2H), 2.29-2.22 (m, 2H), | |
| | | 2.18-2.11 (m, 1H), 2.06 (s, 2H), 2.01-1.93 | |
| | | (m, 3H), 1.91-1.85 (m, 2H), 1.20 | |
| | | (s, 6H), 1.10 (s, 6H) | |
| 217 | 2-[6-[6-[5-acetyl-3-[7- | (DMSO-d6) δ = 8.73 (d, J = 2.8 Hz, 2H), | 1027.6 |
| | (difluoromethyl)-6-(1- | 8.13 (s, 1H), 7.90 (d, J = 8.8 Hz, 1H), | |
| | methylpyrazol-4-yl)-3,4-dihydro- | 7.78-7.70 (m, 2H), 7.50 (s, 1H), 7.21 | |
| | 2H-quinolin-1-yl]-6,7-dihydro- | (d, J = 2.0 Hz, 1H), 7.11 (s, 1H), 7.03-6.98 | |
| | 4H-pyrazolo[4,3-c]pyridin-1-yl]- | (m, 1H), 6.96-6.63 (m, 2H), 4.72-4.60 | |
| | 2-azaspiro[3.3]heptane-2- | (m, 1H), 4.28 (s, 1H), 4.19-4.08 | |
| | carbonyl]-2-azaspiro[3.3]heptan- | (m, 5H), 4.07-3.99 (m, 4H), 3.96 (s, | |
| | 2-yl]-N-[3-(3-chloro-4-cyano- | 1H), 3.89-3.82 (m, 4H), 3.74-3.58 (m, | |
| | phenoxy)-2,2,4,4-tetramethyl- | 4H), 3.04-2.93 (m, 1H), 2.87-2.77 (m, | |
| | cyclobutyl]pyrimidine-5- | 3H), 2.70-2.60 (m, 5H), 2.33 (br t, J = | |
| | carboxamide | 8.8 Hz, 4H), 2.09-2.04 (m, 2H), 2.01-1.92 | |
| | | (m, 3H), 1.21 (s, 6H), 1.10 (s, 6H) | |
| 218 | 2-[6-[6-[5-acetyl-3-[7- | (DMSO-d6) δ = 8.73 (d, J = 2.4 Hz, 2H), | 947.4 |
| | (difluoromethyl)-3,4-dihydro- | 8.13 (s, 1H), 7.90 (d, J = 8.8 Hz, 1H), | |
| | 2H-quinolin-1-yl]-6,7-dihydro- | 7.72 (br d, J = 8.8 Hz, 1H), 7.21 (d, J = | |
| | 4H-pyrazolo[4,3-c]pyridin-1-yl]- | 2.4 Hz, 1H), 7.12 (d, J = 7.6 Hz, 1H), | |
| | 2-azaspiro[3.3]heptane-2- | 7.03-6.97 (m, 1H), 6.95-6.62 (m, 3H), | |
| | carbonyl]-2-azaspiro[3.3]heptan- | 4.70-4.59 (m, 1H), 4.28 (s, 1H), 4.14 | |
| | 2-yl]-N-[3-(3-chloro-4-cyano- | (br d, J = 7.6 Hz, 3H), 4.09 (br s, 1H), | |
| | phenoxy)-2,2,4,4-tetramethyl- | 4.07-4.04 (m, 2H), 4.01 (br d, J = 3.2 | |
| | cyclobutyl]pyrimidine-5- | Hz, 2H), 3.95 (s, 1H), 3.85 (s, 1H), 3.74-3.66 | |
| | carboxamide | (m, 2H), 3.59 (br d, J = 6.0 Hz, | |
| | | 2H), 3.03-2.91 (m, 1H), 2.90-2.71 (m, | |
| | | 4H), 2.64 (br d, J = 8.0 Hz, 4H), 2.34 (br | |
| | | t, J = 8.8 Hz, 4H), 2.08-2.05 (m, 2H), | |
| | | 1.99-1.91 (m, 3H), 1.79-1.72 (m, 1H), | |
| | | 1.21 (s, 6H), 1.11 (s, 6H) | |
| 219 | 2-[6-[[6-[5-acetyl-3-[7- | (DMSO-d₆) δ = 8.72 (s, 2H), 7.89 (d, J = | 933.5 |
| | (difluoromethyl)-3,4-dihydro- | 8.8 Hz, 1H), 7.71 (d, J = 9.2 Hz, 1H), | |
| | 2H-quinolin-1-yl]-6,7-dihydro- | 7.21 (d, J = 2.4 Hz, 1H), 7.11 (d, J = 7.6 | |
| | 4H-pyrazolo[4,3-c]pyridin-1-yl]- | Hz, 1H), 7.03-6.99 (m, 1H), 6.97-6.67 | |
| | 2-azaspiro[3.3]heptan-2- | (m, 2H), 6.65 (br d, J = 6.4 Hz, 1H), | |
| | yl]methyl]-2- | 4.58 (br t, J = 8.0 Hz, 1H), 4.28 (s, 1H), | |
| | azaspiro[3.3]heptan-2-yl]-N-[3- | 4.11 (s, 2H), 4.09-4.01 (m, 3H), 4.01-3.97 | |
| | (3-chloro-4-cyano-phenoxy)- | (m, 2H), 3.73-3.65 (m, 2H), 3.58 | |
| | 2,2,4,4-tetramethyl- | (br d, J = 6.0 Hz, 2H), 3.23 (br s, 2H), | |
| | cyclobutyl]pyrimidine-5- | 3.12 (br s, 2H), 2.86-2.74 (m, 3H), 2.65 | |
| | carboxamide | (br t, J = 5.6 Hz, 1H), 2.61-2.51 (m, | |
| | | 4H), 2.38 (br d, J = 6.8 Hz, 2H), 2.30-2.21 | |
| | | (m, 2H), 2.18-2.09 (m, 1H), 2.07-2.04 | |
| | | (m, 2H), 2.00-1.84 (m, 5H), 1.20 | |
| | | (s, 6H), 1.11 (s, 6H) | |
| 220 | 2-[6-[6-[5-acetyl-3-(6-cyano-3,4- | (DMSO-d₆) δ = 8.73 (d, J = 2.8 Hz, 2H), | 922.4 |
| | dihydro-2H-quinolin-1-yl)-6,7- | 7.90 (d, J = 8.8 Hz, 1H), 7.76-7.67 (m, | |
| | dihydro-4H-pyrazolo[4,3- | 1H), 7.41 (s, 1H), 7.34-7.25 (m, 1H), | |
| | c]pyridin-1-yl]-2- | 7.23-7.16 (m, 1H), 7.03-6.94 (m, 1H), | |
| | azaspiro[3.3]heptane-2- | 6.49-6.35 (m, 1H), 4.75-4.58 (m, 1H), | |
| | carbonyl]-2-azaspiro[3.3]heptan- | 4.28 (s, 1H), 4.19-4.08 (m, 5H), 4.07-3.98 | |
| | 2-yl]-N-[3-(3-chloro-4-cyano- | (m, 4H), 3.96 (s, 1H), 3.85 (s, 1H), | |
| | phenoxy)-2,2,4,4-tetramethyl- | 3.76-3.56 (m, 4H), 3.05-2.90 (m, 1H), | |
| | cyclobutyl]pyrimidine-5- | 2.85-2.59 (m, 8H), 2.39-2.28 (m, 4H), | |
| | carboxamide | 2.06 (s, 2H), 2.01-1.88 (m, 3H), 1.21 | |
| | | (s, 6H), 1.10 (s, 6H). | |
| 221 | 2-[6-[[6-[5-acetyl-3-(6-cyano- | (MeOD) δ = 8.71 (s, 2H), 7.72 (d, J = | 908.7 |
| | 3,4-dihydro-2H-quinolin-1-yl)- | 8.8 Hz, 1H), 7.37-7.32 (m, 1H), 7.29-7.21 | |
| | 6,7-dihydro-4H-pyrazolo[4,3- | (m, 1H), 7.15-7.10 (m, 1H), 7.01-6.94 | |
| | c]pyridin-1-yl]-2- | (m, 1H), 6.51-6.40 (m, 1H), 4.76-4.64 | |
| | azaspiro[3.3]heptan-2- | (m, 1H), 4.29-4.17 (m, 5H), 4.13 | |
| | yl]methyl]-2- | (s, 1H), 4.11-4.05 (m, 2H), 3.88-3.78 | |
| | azaspiro[3.3]heptan-2-yl]-N-[3- | (m, 2H), 3.75-3.64 (m, 4H), 3.64-3.54 | |
| | (3-chloro-4-cyano-phenoxy)- | (m, 2H), 3.37-3.33 (m, 1H), 2.89-2.81 | |
| | 2,2,4,4-tetramethyl- | (m, 3H), 2.79-2.70 (m, 6H), 2.43-2.31 | |
| | cyclobutyl]pyrimidine-5- | (m, 3H), 2.17 (s, 2H), 2.11-2.00 (m, | |
| | carboxamide | 5H), 1.27 (s, 6H), 1.21 (s, 6H). | |
| 224 | 2-[6-[[4-[8-(5-acetyl-1-methyl- | δ = 10.04-9.46 (m, 1H), 8.70 (s, 2H), | 864.7 |
| | 6,7-dihydro-4H-pyrazolo[4,3- | 8.10 (s, 1H), 7.96-7.75 (m, 2H), 7.59 | |
| | c]pyridin-3-yl)-3- | (d, J = 8.6 Hz, 1H), 7.27-7.15 (m, 1H), | |
| | isoquinolyl]pyrazol-1-yl]methyl]- | 6.98 (d, J = 2.4 Hz, 1H), 6.82 (dd, J = | |
| | 2-azaspiro[3.3]heptan-2-yl]-N- | 2.2, 8.8 Hz, 1H), 6.04-5.84 (m, 1H), | |
| | [3-(3-chloro-4-cyano-phenoxy)- | 4.66 (s, 1H), 4.48 (s, 1H), 4.31-4.19 | |
| | 2,2,4,4-tetramethyl- | (m, 4H), 4.16-4.09 (m, 3H), 4.07-4.00 | |
| | cyclobutyl]pyrimidine-5- | (m, 2H), 3.92 (d, J = 4.8 Hz, 3H), 3.86 | |
| | carboxamide | (br t, J = 5.8 Hz, 1H), 2.93-2.82 (m, | |
| | | 2H), 2.54-2.42 (m, 2H), 2.29-2.15 (m, | |
| | | 3H), 2.13-2.04 (m, 2H), 1.56-1.38 (m, | |
| | | 2H), 1.24 (d, J = 11.9 Hz, 13H) | |
| 225 | 2-[6-[2-[4-[8-(5-acetyl-1- | δ = 9.86-9.62 (m, 1H), 8.67 (s, 2H), | 948.4 |
| | tetrahydropyran-4-yl-6,7- | 8.07 (s, 2H), 7.89-7.76 (m, 2H), 7.69-7.73 | |
| | dihydro-4H-pyrazolo[4,3- | (m, 1H), 7.57 (d, J = 8.8 Hz, 1H), | |
| | c]pyridin-3-yl)-3- | 7.54-7.43 (m, 1H), 6.96 (d, J = 2.4 Hz, | |
| | isoquinolyl]pyrazol-1-yl]ethyl]- | 1H), 6.78-6.81 (m, 1H), 5.90-5.94 (m, | |
| | 2-azaspiro[3.3]heptan-2-yl]-N- | 1H), 4.70-4.45 (m, 2H), 4.35-4.23 (m, | |
| | [3-(3-chloro-4-cyano-phenoxy)- | 1H), 4.22-4.13 (m, 6H), 4.12-4.06 (m, | |
| | 2,2,4,4-tetramethyl- | 3H), 4.05-3.99 (m, 2H), 3.83 (t, J = 5.6 | |
| | cyclobutyl]pyrimidine-5- | Hz, 1H), 3.57 (t, J = 12.4 Hz, 2H), 2.97-2.81 | |
| | carboxamide | (m, 2H), 2.51-2.34 (m, 4H), 2.27-2.19 | |
| | | (m, 2H), 2.14-2.01 (m, 4H), 1.98-1.85 | |
| | | (m, 4H), 1.24 (s, 6H), 1.21 (s, 6H) | |
| 226 | 2-[6-[2-[4-[8-(5-acetyl-1-methyl- | δ = 9.79-9.54 (m, 1H), 8.67 (d, J = 0.8 | 878.5 |
| | 6,7-dihydro-4H-pyrazolo[4,3- | Hz, 2H), 8.07 (s, 2H), 7.89-7.77 (m, | |
| | c]pyridin-3-yl)-3- | 2H), 7.76-7.67 (m, 1H), 7.57 (d, J = 8.4 | |
| | isoquinolyl]pyrazol-1-yl]ethyl]- | Hz, 1H), 7.55-7.47 (m, 1H), 6.96 (d, | |
| | 2-azaspiro[3.3]heptan-2-yl]-N- | J = 2.4 Hz, 1H), 6.84-6.75 (m, 1H), 6.01-5.84 | |
| | [3-(3-chloro-4-cyano-phenoxy)- | (s, 1H), 4.63 (s, 1H), 4.45 (s, 1H), | |
| | 2,2,4,4-tetramethyl- | 4.35-4.14 (m, 4H), 4.14-4.07 (m, 3H), | |
| | cyclobutyl]pyrimidine-5- | 4.07-3.92 (m, 3H), 3.92-3.86 (m, 3H), | |
| | carboxamide | 3.86-3.75 (m, 1H), 2.93-2.85 (m, 1H), | |
| | | 2.85-2.77 (m, 1H), 2.47-2.35 (m, 2H), | |
| | | 2.29-2.22 (m, 1H), 2.21 (s, 1H), 2.09-2.04 | |
| | | (m, 3H), 1.94-1.84 (m, 2H), 1.24 | |
| | | (s, 6H), 1.21 (s, 2H). | |
| 227 | 2-[6-[2-[5-[8-(5-acetyl-1- | δ = 9.83 (d, J = 6.8 Hz, 1H), 9.23 (s, | 959.4 |
| | tetrahydropyran-4-yl-6,7- | 1H), 8.68 (s, 2H), 8.43-8.34 (m, 1H), | |
| | dihydro-4H-pyrazolo[4,3- | 8.24 (s, 1H), 8.10 (d, J = 11.1 Hz, 1H), | |
| | c]pyridin-3-yl)-3-isoquinolyl]-2- | 7.87-7.93 (m, 1H), 7.72-7.79 (m, 1H), | |
| | pyridyl]ethyl]-2- | 7.63-7.53 (m, 2H), 7.33-7.28 (m, 1H), | |
| | azaspiro[3.3]heptan-2-yl]-N-[3- | 6.96 (d, J = 2.3 Hz, 1H), 6.78-6.81 (m, | |
| | (3-chloro-4-cyano-phenoxy)- | 1H), 5.93 (d, J = 8.4 Hz, 1H), 4.72-4.44 | |
| | 2,2,4,4-tetramethyl- | (m, 2H), 4.35-4.25 (m, 1H), 4.24-4.14 | |
| | cyclobutyl]pyrimidine-5- | (m, 4H), 4.13-4.07 (m, 3H), 4.06-3.99 | |
| | carboxamide | (m, 2H), 3.86 (t, J = 5.6 Hz, 1H), 3.54-3.60 | |
| | | (m, 2H), 2.94 (t, J = 5.2 Hz, 1H), | |
| | | 2.90-2.78 (m, 3H), 2.53-2.35 (m, 4H), | |
| | | 2.24-2.32 (m, 1H), 2.21 (s, 1H), 2.08 | |
| | | (s, 2H), 1.98-1.84 (m, 6H), 1.24 (s, | |
| | | 6H), 1.21 (s, 6H) | |
| 228 | 2-[6-[[5-[8-(5-acetyl-1- | δ = 9.84 (d, J = 7.6 Hz, 1H), 9.25 (s, | 945.6 |
| | tetrahydropyran-4-yl-6,7- | 1H), 8.69 (s, 2H), 8.42 (m, 1H), 8.11 (d, | |
| | dihydro-4H-pyrazolo[4,3- | J = 10.0 Hz, 1H), 7.98-7.70 (m, 2H), | |
| | c]pyridin-3-yl)-3-isoquinolyl]-2- | 7.67-7.54 (m, 2H), 7.29 (s, 1H), 6.96 | |
| | pyridyl]methyl]-2- | (d, J = 2.4 Hz, 1H), 6.85-6.74 (m, 1H), | |
| | azaspiro[3.3]heptan-2-yl]-N-[3- | 5.92 (m, 1H), 4.67 (s, 1H), 4.51 (s, 1H), | |
| | (3-chloro-4-cyano-phenoxy)- | 4.36-4.21 (m, 3H), 4.21-4.09 (m, 5H), | |
| | 2,2,4,4-tetramethyl- | 4.06-3.99 (m, 2H), 3.86 (m, 1H), 3.58 | |
| | cyclobutyl]pyrimidine-5- | (m, 2H), 3.02 (m, 2H), 2.94 (m, 1H), | |
| | carboxamide | 2.87 (mz, 1H), 2.82-2.69 (m, 1H), 2.53-2.37 | |
| | | (m, 4H), 2.23-2.21 (m, 1H), 2.17-2.05 | |
| | | (m, 4H), 1.98 (m, 2H), 1.24 (s, | |
| | | 6H), 1.21 (s, 6H) | |
| 229 | 2-[6-[2-[5-[8-(5-acetyl-1-methyl- | δ = 9.78 (d, J = 12.8 Hz, 1H), 9.25 (s, | 889.7 |
| | 6,7-dihydro-4H-pyrazolo[4,3- | 1H), 8.68 (s, 2H), 8.41 (br d, J = 6.4 Hz, | |
| | c]pyridin-3-yl)-3-isoquinolyl]-2- | 1H), 8.11 (d, J = 10.8 Hz, 1H), 7.91 (dd, | |
| | pyridyl]ethyl]-2- | J = 8.4, 14.8 Hz, 1H), 7.76 (td, J = 8.4, | |
| | azaspiro[3.3]heptan-2-yl]-N-[3- | 12.4 Hz, 1H), 7.64-7.53 (m, 2H), 7.33-7.28 | |
| | (3-chloro-4-cyano-phenoxy)- | (m, 1H), 6.96 (d, J = 2.4 Hz, 1H), | |
| | 2,2,4,4-tetramethyl- | 6.80 (dd, J = 2.4, 8.8 Hz, 1H), 5.92 (d, | |
| | cyclobutyl]pyrimidine-5- | J = 8.4 Hz, 1H), 4.63 (s, 1H), 4.47 (s, 1H), | |
| | carboxamide | 4.21 (s, 2H), 4.14-4.07 (m, 3H), 4.06-3.99 | |
| | | (m, 2H), 3.91 (d, J = 2.8 Hz, 3H), | |
| | | 3.85 (t, J = 6.0 Hz, 1H), 2.90 (br t, J = | |
| | | 5.6 Hz, 1H), 2.83 (br t, J = 7.6 Hz, 3H), | |
| | | 2.44-2.35 (m, 2H), 2.29 (td, J = 7.2, | |
| | | 14.8 Hz, 1H), 2.21-2.05 (m, 3H), 1.97-1.88 | |
| | | (m, 4H), 1.23 (d, J = 12.0 Hz, 12H). | |
| 230 | 2-[6-[[5-[8-(5-acetyl-1-methyl- | δ = 9.78 (d, J = 12.00 Hz, 1 H) 9.25 (br s, | 875.6 |
| | 6,7-dihydro-4H-pyrazolo[4,3- | 1 H) 8.69 (s, 2 H) 8.40 (br d, J = 5.60 Hz, | |
| | c]pyridin-3-yl)-3-isoquinolyl]-2- | 1 H) 8.11 (d, J = 11.20 Hz, 1 H) 7.91 (dd, | |
| | pyridyl]methyl]-2- | J = 14.80, 8.40 Hz, 1 H) 7.76 (dt, | |
| | azaspiro[3.3]heptan-2-yl]-N-[3- | J = 12.40, 7.60 Hz, 1 H) 7.59 (br s, 1 H) | |
| | (3-chloro-4-cyano-phenoxy)- | 7.57 (d, J = 8.40 Hz, 1 H) 6.96 (d, J = 1.60 | |
| | 2,2,4,4-tetramethyl- | Hz, 1 H) 6.80 (dd, J = 8.80, 1.60 Hz, 1 H) | |
| | cyclobutyl]pyrimidine-5- | 5.92 (d, J = 8.00 Hz, 1 H) 4.64 (s, 1 H) | |
| | carboxamide | 4.47 (s, 1 H) 4.23 (s, 2 H) 4.09-4.17 | |
| | | (m, 3 H) 3.98-4.06 (m, 2 H) 3.92 (d, | |
| | | J = 2.40 Hz, 3 H) 3.85 (br t, J = 5.60 Hz, | |
| | | 1 H) 3.00 (br d, J = 7.20 Hz, 2 H) 2.90 (br t, | |
| | | J = 5.60 Hz, 1 H) 2.84 (br t, J = 5.60 Hz, | |
| | | 1 H) 2.75 (dt, J = 15.60, 7.60 Hz, 1 H) 2.41 | |
| | | (br t, J = 10.00 Hz, 2 H) 2.21 (s, 1 H) 2.12 | |
| | | (br d, J = 9.20 Hz, 2 H) 2.04-2.08 (m, | |
| | | 2 H) 1.24 (s, 6 H) 1.21 (s, 6 H). | |

### Example 25: Synthesis of N-[3-(3-chloro-4-cyano-phenoxy)-2,2,4,4-tetramethyl-cyclobutyl]-2-[4-[2-[5-[8-(1,3-dimethyl-2-oxo-benzimidazol-5-yl)-3-isoquinolyl]-2-pyridyl]ethyl]-1-piperidyl]pyrimidine-5-carboxamide (Compound 232)

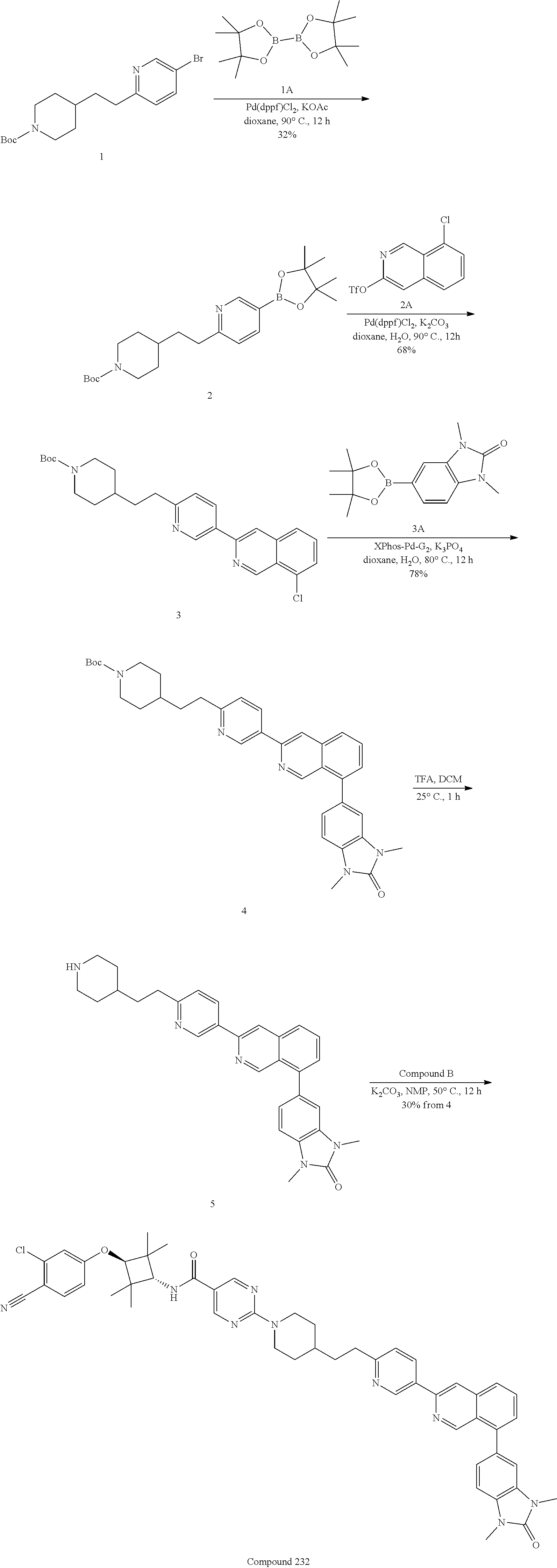

Synthesis of 1 was reported in WO2023/38500 A1.

Synthesis of 2A was reported in Dalton Transactions, 2008, vol. 8, #4, p. 491-498.

Synthesis of 3A was reported in WO2017/205536 A2.

To a solution of tert-butyl 4-[2-(5-bromo-2-pyridyl)ethyl]piperidine-1-carboxylate (3.80 g, 10.20 mmol, 1.0 eq) in dioxane (72 mL) was added Pd(dppf)Cl₂ (752 mg, 1.00 mmol, 0.1 eq), KOAc (3.03 g, 30.81 mmol, 3.0 eq) and 4,4,5,5-tetramethyl-2-(4,4,5,5-tetramethyl-1,3,2-dioxaborolan-2-yl)-1,3,2-dioxaborolane (5.23 g, 20.50 mmol, 2.0 eq). The mixture was stirred to 90° C. for 12 h under N₂. The reaction mixture was diluted with water (15 mL) and extracted with EtOAc (15 mL*3). The combined organic layers were washed with sat. NaCl (20 mL*3), dried over Na₂SO₄, filtered and concentrated under reduced pressure to give a residue. The residue was purified by silica gel column chromatography (petroleum ether/ethyl acetate=50/1 to 5/1). Compound tert-butyl 4-[2-[5-(4,4,5,5-tetramethyl-1,3,2-dioxaborolan-2-yl)-2-pyridyl]ethyl]piperidine-1-carboxylate (1.40 g, 3.30 mmol, 32% yield) was obtained as a black solid.

LC-MS: MS (ESI⁺): t_{R}=0.808 min, m/z=416.2 [M+H⁺]

To a solution of (8-chloro-3-isoquinolyl) trifluoromethanesulfonate (300 mg, 962 μmol, 1.0 eq) and tert-butyl 4-[2-[5-(4,4,5,5-tetramethyl-1,3,2-dioxaborolan-2-yl)-2-pyridyl]ethyl]piperidine-1-carboxylate (400 mg, 962 mol, 1.0 eq) in dioxane (5 mL) and H₂O (0.5 mL) was added K₂CO₃ (399 mg, 2.80 mmol, 3.0 eq) and Pd(dppf)Cl₂ (70 mg, 96 μmol, 0.1 eq) under N₂ atmosphere. The mixture was stirred at 90° C. for 12 h under N₂ atmosphere. The residue was diluted with H₂O (15 mL) and extracted with EtOAc (10 mL*3). The combined organic layers were washed with sat. NaCl (10 ml*3), dried over Na₂SO₄, filtered and concentrated under reduced pressure to give a residue. The residue was purified by silica gel column chromatography (petroleum ether/ethyl acetate=10/1 to 0/1). Compound tert-butyl 4-[2-[5-(8-chloro-3-isoquinolyl)-2-pyridyl]ethyl]piperidine-1-carboxylate (300 mg, 663 μmol, 68% yield) was obtained as a white solid.

LC-MS: MS (ESI⁺): t_{R}=0.566 min, m/z=451.2 [M+H⁺]

To a solution of tert-butyl 4-[2-[5-(8-chloro-3-isoquinolyl)-2-pyridyl]ethyl]piperidine-1-carboxylate (240 mg, 530 mol, 1.0 eq) and 1,3-dimethyl-5-(4,4,5,5-tetramethyl-1,3,2-dioxaborolan-2-yl)benzimidazol-2-one (198 mg, 690 μmol, 1.3 eq) in dioxane (5 mL) and H₂O (1.5 mL) was added K₃PO₄ (338 mg, 1.50 mmol, 3.0 eq) and XPhos-Pd-G₂ (41 mg, 53 μmol, 0.1 eq) under N₂ atmosphere. The mixture was stirred at 80° C. for 12 h under N₂ atmosphere. The residue was diluted with H₂O (15 ml) and extracted with EA (10 mL*3). The combined organic layers were washed with sat. NaCl (10 mL*3), dried over Na₂SO₄, filtered and concentrated under reduced pressure to give a residue. The residue was purified by silica gel column chromatography (petroleum ether/ethyl acetate=10/1 to 0/1). Compound tert-butyl 4-[2-[5-[8-(1,3-dimethyl-2-oxo-benzimidazol-5-yl)-3-isoquinolyl]-2-pyridyl]ethyl]piperidine-1-carboxylate (240 mg, 415 μmol, 78% yield) was obtained as a white solid.

LC-MS: MS (ESI⁺): t_{R}=0.555 min, m/z=577.3 [M+H⁺]

To a solution of tert-butyl 4-[2-[5-[8-(1,3-dimethyl-2-oxo-benzimidazol-5-yl)-3-isoquinolyl]-2-pyridyl]ethyl]piperidine-1-carboxylate (240 mg, 415 μmol, 1.0 eq) in DCM (3 mL) was added TFA (1.54 g, 13.40 mmol, 1.0 mL, 32.4 eq). The mixture was stirred at 25° C. for 1 h. The reaction mixture was concentrated under reduced pressure to give a crude product. Compound 1,3-dimethyl-5-[3-[6-[2-(4-piperidyl)ethyl]-3-pyridyl]-8-isoquinolyl]benzimidazol-2-one (198 mg) was obtained as a yellow oil and directly used in the next step without further purification.

LC-MS: MS (ESI⁺): t_{R}=0.434 min, m/z=477.2 [M+H⁺]

A mixture of 1,3-dimethyl-5-[3-[6-[2-(4-piperidyl)ethyl]-3-pyridyl]-8-isoquinolyl]benzimidazol-2-one (99 mg), 2-chloro-N-[3-(3-chloro-4-cyano-phenoxy)-2,2,4,4-tetramethyl-cyclobutyl]pyrimidine-5-carboxamide (86 mg, 207 mol, 1.0 eq) and K₂CO₃ (85 mg, 621 mol, 3.0 eq) in NMP (1.5 mL) was stirred at 50° C. for 12 h. The reaction mixture was filtered and concentrated under reduced pressure to give a residue. The residue was purified by prep-HPLC (column: Phenomenex luna C18 150*25 mm*10 um; mobile phase: [water (FA)-ACN]; gradient: 58%-88% B over 10 min). Compound N-[3-(3-chloro-4-cyano-phenoxy)-2,2,4,4-tetramethyl-cyclobutyl]-2-[4-[2-[5-[8-(1,3-dimethyl-2-oxo-benzimidazol-5-yl)-3-isoquinolyl]-2-pyridyl]ethyl]-1-piperidyl]pyrimidine-5-carboxamide (55 mg, 30% yield over two steps) was obtained as a white solid.

¹H NMR (400 MHz, DMSO-d₆) δ=9.38-9.27 (m, 2H), 8.75 (s, 2H), 8.57 (s, 1H), 8.47 (s, 1H), 8.04 (s, 1H), 7.92-7.83 (m, 2H), 7.73-7.58 (m, 2H), 7.47-7.40 (m, 2H), 7.36-7.32 (m, 1H), 7.30-7.26 (m, 1H), 7.21 (m, 1H), 7.00 (m, 1H), 4.75 (m, 2H), 4.28 (s, 1H), 4.03 (m, 1H), 3.41 (s, 6H), 2.98-2.90 (m, 2H), 2.89-2.82 (m, 2H), 1.85 (m, 2H), 1.73-1.58 (m, 3H), 1.21 (s, 6H), 1.16-1.05 (m, 8H).

LC-MS: MS (ESI⁺): t_{R}=2.298 min, m/z=859.3 [M+H⁺]

### Example 26: Synthesis of N-[3-(3-chloro-4-cyano-phenoxy)-2,2,4,4-tetramethyl-cyclobutyl]-2-[4-[2-[5-[1-(1-methyl-2-oxo-3,4-dihydroquinolin-6-yl)-3,4-dihydro-2H-1,7-naphthyridin-6-yl]-2-pyridyl]ethyl]-1-piperidyl]pyrimidine-5-carboxamide (Compound 240)

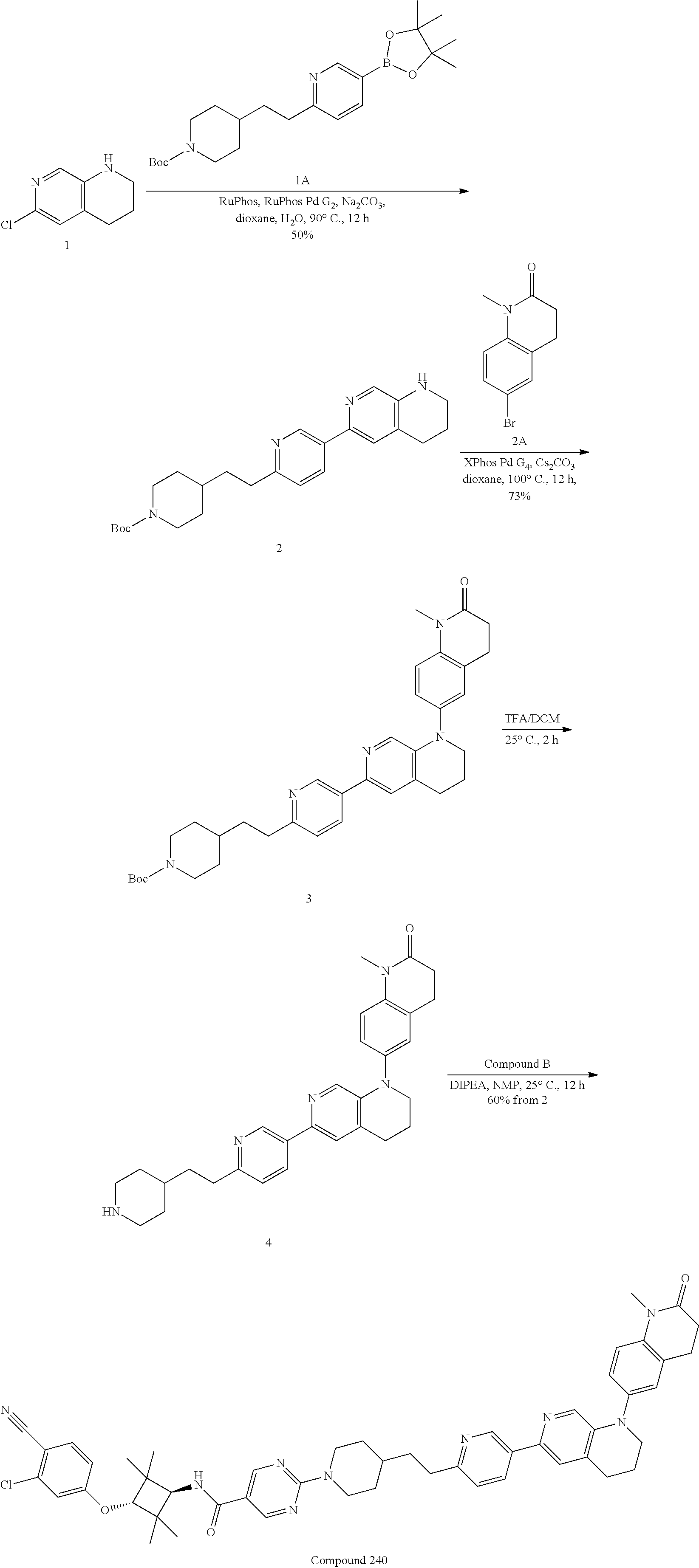

Synthesis of 1 was reported in WO2022/20342 A1.

To a solution of 6-chloro-1,2,3,4-tetrahydro-1,7-naphthyridine (200 mg, 1.19 mmol, 1.0 eq), tert-butyl 4-[2-[5-(4,4,5,5-tetramethyl-1,3,2-dioxaborolan-2-yl)-2-pyridyl]ethyl]piperidine-1-carboxylate (1.33 g, 3.20 mmol, 2.7 eq), dicyclohexyl-[2-(2,6-diisopropoxyphenyl)phenyl]phosphane (55 mg, 119 μmol, 0.1 eq), [2-(2-aminophenyl)phenyl]-chloro-palladium; dicyclohexyl-[2-(2,6-diisopropoxyphenyl)phenyl]phosphane (92 mg, 119 μmol, 0.1 eq) in dioxane (6.5 mL) was added Na₂CO₃ (2 M, 1.8 mL, 3.0 eq) in H₂O (1.8 mL) under N₂. The mixture was stirred at 90° C. for 12 h. The reaction mixture was poured into ice water 50 mL at 0° C., and then extracted with DCM (20 mL×3). The combined organic layers were washed with brine 20 mL, dried over Na₂SO₄, filtered and concentrated under reduced pressure to give a residue. The residue was purified by column chromatography (SiO₂, Petroleum ether/Ethyl acetate=10/1 to I/O). Compound tert-butyl 4-[2-[5-(1,2,3,4-tetrahydro-1,7-naphthyridin-6-yl)-2-pyridyl]ethyl]piperidine-1-carboxylate (253 mg, 599 μmol, 50% yield) was obtained as a yellow solid.

LC-MS: MS (ESI⁺): t_{R}=0.553 min, m/z=582.6 [M+H⁺]

To a solution of tert-butyl 4-[2-[5-(1,2,3,4-tetrahydro-1,7-naphthyridin-6-yl)-2-pyridyl]ethyl]piperidine-1-carboxylate (200 mg, 473 μmol, 1.0 eq), 6-bromo-1-methyl-3,4-dihydroquinolin-2-one (140 mg, 583 μmol, 1.2 eq), Xphos Pd G4 (40 mg, 46.5 μmol, 1.0 eq), Cs₂CO₃ (312 mg, 958 μmol, 2.0 eq) in dioxane (8 mL). The mixture was stirred at 100° C. for 12 h under N₂. The reaction mixture was poured into ice/water 50 mL at 0° C., and then extracted with DCM (20 mL×3). The combined organic layers were washed with brine 20 mL, dried over Na₂SO₄, filtered and concentrated under reduced pressure to give a residue. The crude product was purified by reversed-phase HPLC (column: Phenomenex Luna C18 150×25 mm×10 um; mobile phase: [water (FA)-ACN]; gradient: 37%-67% B over 10 min). Compound tert-butyl 4-[2-[5-[1-(1-methyl-2-oxo-3,4-dihydroquinolin-6-yl)-3,4-dihydro-2H-1,7-naphthyridin-6-yl]-2-pyridyl]ethyl]piperidine-1-carboxylate (201 mg, 346 mol, 73% yield) was obtained as a yellow solid.

LC-MS: MS (ESI⁺): t_{R}=0.554 min, m/z=582.6 [M+H⁺]

To a solution of tert-butyl 4-[2-[5-[1-(1-methyl-2-oxo-3,4-dihydroquinolin-6-yl)-3,4-dihydro-2H-1,7-naphthyridin-6-yl]-2-pyridyl]ethyl]piperidine-1-carboxylate (100 mg, 172 μmol, 1.0 eq) in DCM (1 mL) was added TFA (0.5 mL). The mixture was stirred at 25° C. for 2 h. The reaction mixture was concentrated under reduced pressure to give a residue. Compound 1-methyl-6-[6-[6-[2-(4-piperidyl) ethyl]-3-pyridyl]-3,4-dihydro-2H-1,7-naphthyridin-1-yl]-3,4-dihydroquinolin-2-one (102 mg) was obtained as a yellow solid and directly used in the next step without further purification.

LC-MS: MS (ESI⁺): t_{R}=0.469 min, m/z=482.4 [M+H⁺]

To a solution of 1-methyl-6-[6-[6-[2-(4-piperidyl)ethyl]-3-pyridyl]-3,4-dihydro-2H-1,7-naphthyridin-1-yl]-3,4-dihydroquinolin-2-one (51 mg), 2-chloro-N-[3-(3-chloro-4-cyano-phenoxy)-2,2,4,4-tetramethyl-cyclobutyl]pyrimidine-5-carboxamide (54 mg, 129 μmol, 1.5 eq) in NMP (1 mL) was added DIPEA (33 mg, 257 μmol, 45.0 μL, 3.0 eq). The mixture was stirred at 25° C. for 12 h. The reaction mixture was poured into ice water 50 mL at 0° C., and then extracted with DCM (20 mL×3). The combined organic layers were washed with brine 20 mL, dried over Na₂SO₄, filtered and concentrated under reduced pressure to give a residue. The crude product was purified by reversed-phase HPLC (column: Phenomenex luna C18 150×25 mm×10 um; mobile phase: [water (FA)-ACN]; gradient: 50%-80% B over 10 min). Compound N-[3-(3-chloro-4-cyano-phenoxy)-2,2,4,4-tetramethyl-cyclobutyl]-2-[4-[2-[5-[1-(1-methyl-2-oxo-3,4-dihydroquinolin-6-yl)-3,4-dihydro-2H-1,7-naphthyridin-6-yl]-2-pyridyl]ethyl]-1-piperidyl]pyrimidine-5-carboxamide (44.87 mg, 51.4 μmol, 60% yield over two steps) was obtained as a yellow solid.

¹H NMR (400 MHZ, DMSO-d₆): δ=9.03 (d, J=2.4 Hz, 1H), 8.75 (s, 2H), 8.27 (br d, J=8.0 Hz, 1H), 7.90 (d, J=8.8 Hz, 1H), 7.85 (s, 1H), 7.74-7.66 (m, 2H), 7.39 (br d, J=8.4 Hz, 1H), 7.25-7.19 (m, 3H), 7.18-7.13 (m, 1H), 7.03-6.97 (m, 1H), 4.75 (br d, J=13.2 Hz, 2H), 4.29 (s, 1H), 4.03 (d, J=9.2 Hz, 1H), 3.66-3.58 (m, 2H), 3.27 (s, 3H), 2.99-2.91 (m, 2H), 2.89 (br d, J=4.4 Hz, 4H), 2.86-2.80 (m, 2H), 2.60-2.54 (m, 2H), 2.06-1.97 (m, 2H), 1.83 (br d, J=11.2 Hz, 2H), 1.72-1.54 (m, 3H), 1.21 (s, 6H), 1.11 (s, 8H)

LC-MS: MS (ESI⁺): t_{R}=2.182 min, m/z=864.6 [M+H⁺]

The compounds below were prepared in a similar manner as described in Examples 25 and 26.

| | | | LC-MS |
|---|---|---|---|
| Compound | Name | ¹H NMR (CDCl₃) | [M + H⁺] |
| 233 | N-[3-(3-chloro-4-cyano- | (DMSO-d₆) δ = 8.75 (s, 2H), 8.38 (s, | 913.4 |
| | phenoxy)-2,2,4,4-tetramethyl- | 1H), 8.21-8.11 (m, 1H), 7.90 (m, 1H), | |
| | cyclobutyl]-2-[4-[2-[5-[7- | 7.74-7.55 (m, 2H), 7.34-7.29 (m, 1H), | |
| | (difluoromethyl)-1-(1,3- | 7.25-7.20 (m, 2H), 7.17 (s, 1H), 7.05-6.97 | |
| | dimethyl-2-oxo-benzimidazol- | (m, 3H), 6.75-6.41 (m, 2H), 4.75 | |
| | 5-yl)-3,4-dihydro-2H-quinolin- | (s, 2H), 4.29 (s, 1H), 4.03 (m, 1H), 3.62 | |
| | 6-yl]-2-pyridyl]ethyl]-1- | (s, 2H), 3.36 (s, 5H), 3.00-2.86 (m, 4H), | |
| | piperidyl]pyrimidine-5- | 2.85-2.76 (m, 2H), 2.10-1.99 (m, 2H), | |
| | carboxamide | 1.84 (m, 2H), 1.72-1.58 (m, 3H), 1.21 | |
| | | (s, 7H), 1.11 (s, 8H). | |
| 234 | N-[3-(3-chloro-4-cyano- | (DMSO-d₆) δ = 9.03-8.95 (m, 1H), 8.74 | 865.6 |
| | phenoxy)-2,2,4,4-tetramethyl- | (s, 2H), 8.18-8.12 (m, 1H), 7.92-7.85 | |
| | cyclobutyl]-2-[4-[2-[5-[1-(1,3- | (m, 1H), 7.75-7.61 (m, 3H), 7.30-7.24 | |
| | dimethyl-2-oxo-benzimidazol- | (m, 1H), 7.23-7.15 (m, 3H), 7.04-6.96 | |
| | 5-yl)-3,4-dihydro-2H-1,7- | (m, 2H), 4.82-4.64 (m, 2H), 4.28 (s, | |
| | naphthyridin-6-yl]-2-pyridyl] | 1H), 4.08-3.97 (m, 1H), 3.65-3.59 (m, | |
| | ethyl]-1-piperidyl] pyrimidine- | 2H), 3.58-3.33 (m, 6H), 2.96-2.86 (m, | |
| | 5-carboxamide | 4H), 2.83-2.69 (m, 2H), 2.09-1.99 (m, | |
| | | 2H), 1.88-1.74 (m, 2H), 1.69-1.53 (m, | |
| | | 3H), 1.38-0.93 (m, 14H). | |
| 235 | N-[3-(3-chloro-4-cyano- | δ = 9.41 (s, 1 H) 9.27 (s, 1 H) 8.70 (s, | 863.3 |
| | phenoxy)-2,2,4,4-tetramethyl- | 2 H) 8.42 (m, 1 H) 8.17 (s, 1 H) 7.96 (d, | |
| | cyclobutyl]-2-[4-[2-[5-[8-(3- | J = 8.8 Hz, 1 H) 7.80 (t, J = 7.6 Hz, 1 H) | |
| | methyl-2-oxo-1,3-benzothiazol- | 7.55-7.66 (m, 3 H) 7.31-7.41 (m, 2 H) | |
| | 5-yl)-3-isoquinolyl]-2- | 7.15-7.24 (m, 1 H) 6.97 (d, J = 2.4 Hz, | |
| | pyridyl]ethyl]-1- | 1 H) 6.81 (m, 1 H) 5.92 (m, 1 H) 4.87 (m, | |
| | piperidyl]pyrimidine-5- | 2 H) 4.13 (d, J = 8.0 Hz, 1 H) 4.05 (s, 1 H) | |
| | carboxamide | 3.51 (s, 3 H) 2.89-3.06 (m, 4 H) 1.87-1.98 | |
| | | (m, 2 H) 1.76-1.86 (m, 2 H) 1.66-1.72 | |
| | | (m, 1 H) 1.27-1.34 (m, 2 H) 1.25 | |
| | | (s, 6 H) 1.22 (s, 6 H). | |
| 236 | N-[3-(3-chloro-4-cyano- | δ = 8.71 (s, 2H), 8.52 (d, J = 2.0 Hz, 1H), | 917.5 |
| | phenoxy)-2,2,4,4-tetramethyl- | 7.66-7.54 (m, 2H), 7.45 (d, J = 8.4 Hz, | |
| | cyclobutyl]-2-[4-[2-[5-[7- | 1H), 7.26-7.21 (m, 1H), 7.14-7.07 (m, | |
| | (difluoromethyl)-1-(3-methyl-2- | 1H), 7.04 (d, J = 8.0 Hz, 2H), 7.02-6.94 | |
| | oxo-1,3-benzothiazol-5-yl)-3,4- | (m, 2H), 6.85-6.76 (m, 1H), 6.52-6.19 | |
| | dihydro-2H-quinolin-6-yl]-2- | (m, 1H), 5.92 (d, J = 8.3 Hz, 1H), 4.88 | |
| | pyridyl]ethyl]-1- | (m, 2H), 4.13 (d, J = 8.4 Hz, 1H), 4.05 (s, | |
| | piperidyl]pyrimidine-5- | 1H), 3.77-3.64 (m, 2H), 3.43 (s, 3H), | |
| | carboxamide | 3.06-2.82 (m, 6H), 2.20-2.08 (m, 2H), | |
| | | 1.97-1.86 (m, 2H), 1.82-1.67 (m, 3H), | |
| | | 1.65-1.58 (m, 2H), 1.50-1.50 (m, 1H), | |
| | | 1.26 (s, 6H), 1.22 (s, 6H) | |
| 237 | N-[3-(3-chloro-4-cyano- | δ = 9.07 (s, 1H), 8.70 (s, 1H), 8.77-8.64 | 868.5 |
| | phenoxy)-2,2,4,4-tetramethyl- | (m, 1H), 8.54-8.18 (m, 1H), 8.13-7.99 | |
| | cyclobutyl]-2-[4-[2-[5-[1-(3- | (m, 1H), 7.58 (d, J = 8.8 Hz, 1H), 7.50-7.42 | |
| | methyl-2-oxo-1,3-benzothiazol- | (m, 2H), 7.41-7.32 (m, 1H), 7.27-7.21 | |
| | 5-yl)-3,4-dihydro-2H-1,7- | (m, 1H), 7.14-7.07 (m, 1H), 6.97 | |
| | naphthyridin-6-yl]-2- | (d, J = 2.4 Hz, 2H), 6.83-6.77 (m, 1H), | |
| | pyridyl]ethyl]-1- | 5.94 (d, J = 8.0 Hz, 1H), 4.86 (m, 2H), | |
| | piperidyl]pyrimidine-5- | 4.18-4.10 (m, 1H), 4.08-4.02 (m, 1H), | |
| | carboxamide | 3.77-3.69 (m, 2H), 3.45-3.37 (m, 3H), | |
| | | 3.05-2.87 (m, 6H), 2.85 (s, 1H), 2.42-2.34 | |
| | | (m, 1H), 2.20-2.11 (m, 2H), 2.09-1.97 | |
| | | (m, 1H), 1.94-1.85 (m, 2H), 1.83-1.73 | |
| | | (m, 2H), 1.25 (s, 6H), 1.22 (s, 6H) | |
| 238 | N-[3-(3-chloro-4-cyano- | (DMSO-d₆): δ = 8.79 (s, 2H), 7.90 (d, J = | 1004.8 |
| | phenoxy)-2,2,4,4-tetramethyl- | 8.8 Hz, 1H), 7.80-7.71 (m, 2H), 7.50 (s, | |
| | cyclobutyl]-2-[4-[2-[5-[8-(1- | 1H), 7.21 (d, J = 2.4 Hz, 1H), 7.10 (s, | |
| | methyl-2-oxo-3,4- | 1H), 7.00 (d, J = 2.4, 8.8 Hz, 1H), 6.96-6.62 | |
| | dihydroquinolin-6-yl)-3- | (m, 2H), 4.33-4.22 (m, 2H), 4.19-4.10 | |
| | isoquinolyl]-2-pyridyl]ethyl]-1- | (m, 2H), 4.04 (d, J = 9.2 Hz, 1H), | |
| | piperidyl]pyrimidine-5- | 3.86 (s, 7H), 3.77-3.66 (m, 4H), 3.61-3.55 | |
| | carboxamide | (m, 2H), 3.29-3.23 (m, 4H), 3.00-2.90 | |
| | | (m, 2H), 2.89-2.71 (m, 4H), 2.10-2.04 | |
| | | (m, 2H), 2.02 (s, 7H), 1.21 (s, 6H), | |
| | | 1.11 (s, 6H) | |
| 239 | N-[3-(3-chloro-4-cyano- | (DMSO-d₆): δ = 8.75 (s, 2H), 8.39 (d, J = | 913.7 |
| | phenoxy)-2,2,4,4-tetramethyl- | 2.4 Hz, 1H), 7.90 (d, J = 8.8 Hz, 1H), | |
| | cyclobutyl]-2-[4-[2-[5-[7- | 7.71 (d, J = 9.2 Hz, 1H), 7.61 (d, J = 2.3, | |
| | (difluoromethyl)-1-(1-methyl-2- | 8.0 Hz, 1H), 7.32 (d, J = 8.0 Hz, 1H), | |
| | oxo-3,4-dihydroquinolin-6-yl)- | 7.23-7.14 (m, 4H), 7.05 (s, 1H), 7.01 (d, | |
| | 3,4-dihydro-2H-quinolin-6-yl]- | J = 2.3, 8.8 Hz, 1H), 6.77-6.48 (m, 2H), | |
| | 2-pyridyl]ethyl]-1- | 4.75 (d, J = 13.6 Hz, 2H), 4.29 (s, 1H), | |
| | piperidyl]pyrimidine-5- | 4.03 (d, J = 9.2 Hz, 1H), 3.59 (t, J = 5.2 | |
| | carboxamide | Hz, 2H), 3.27 (s, 3H), 2.99-2.91 (m, | |
| | | 2H), 2.91-2.85 (m, 4H), 2.84-2.79 (m, | |
| | | 2H), 2.59-2.54 (m, 2H), 2.04-1.97 (m, | |
| | | 2H), 1.84 (d, J = 12.0 Hz, 2H), 1.70-1.58 | |
| | | (m, 3H), 1.21 (s, 6H), 1.15 (s, 2H), | |
| | | 1.11 (s, 6H) | |

### Example 27: Synthesis of N-[3-(3-chloro-4-cyano-phenoxy)-2,2,4,4-tetramethyl-cyclobutyl]-2-[4-[[4-[1,3-dimethyl-6-[3-(1-methylpyrazol-4-yl)-8-isoquinolyl]-2-oxo-benzimidazol-4-yl]-1-piperidyl]methyl]-1-piperidyl]pyrimidine-5-carboxamide (Compound 213)

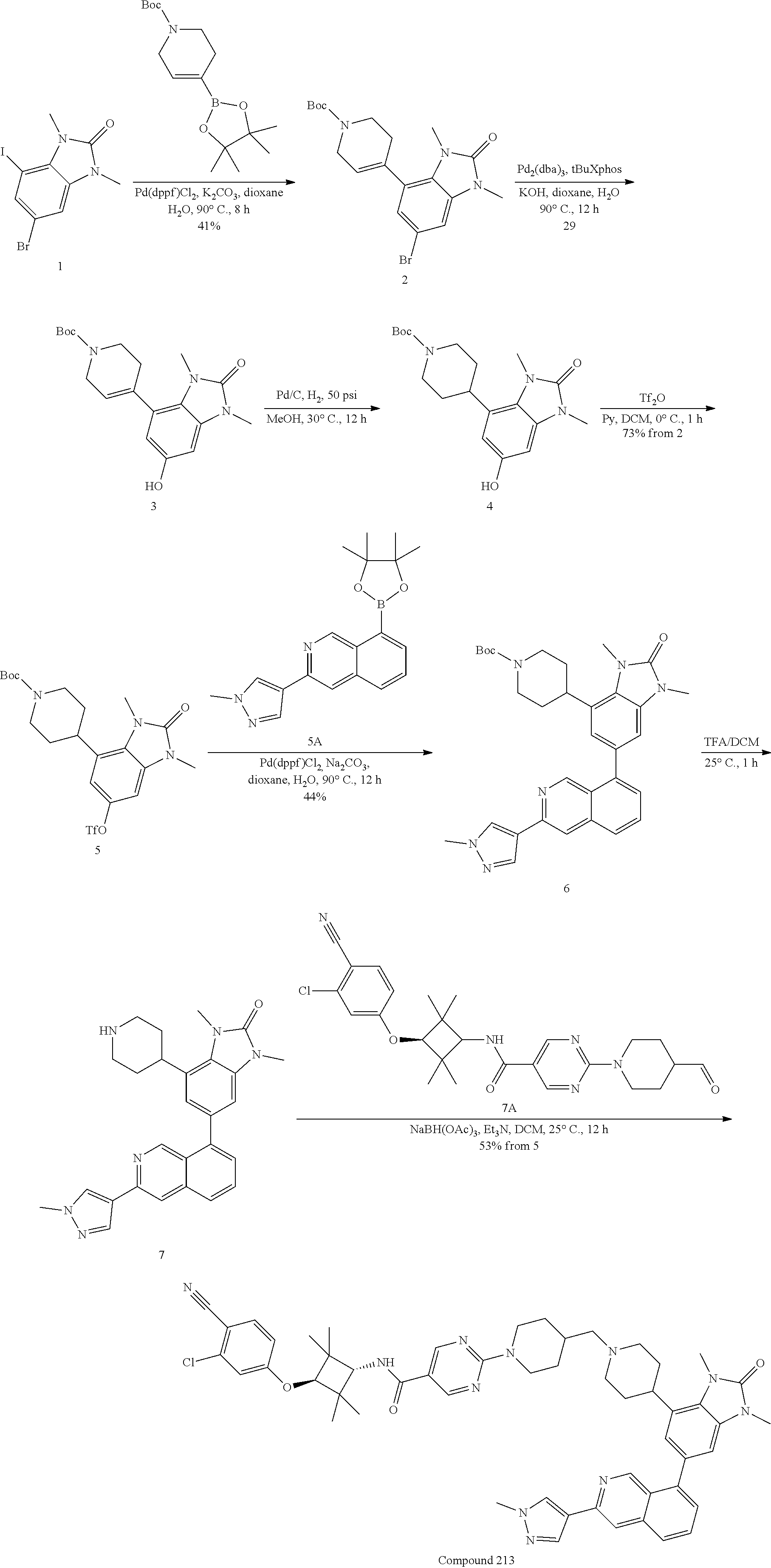

Synthesis of 1 was reported in Journal of Organic Chemistry, 2018, vol. 83, #17, p. 10627-10635

Synthesis of 5A was reported in US2019/308978 A1

Synthesis of 7A was reported in WO2021/249534 A1

To a solution of 6-bromo-4-iodo-1,3-dimethyl-benzimidazol-2-one (4.00 g, 10.90 mmol, 1.0 eq) and tert-butyl 4-(4,4,5,5-tetramethyl-1,3,2-dioxaborolan-2-yl)-3,6-dihydro-2H-pyridine-1-carboxylate (6.74 g, 21.80 mmol, 2.0 eq) in dioxane (82 mL) and H₂O (16 mL) was added K₂CO₃ (3.77 g, 27.25 mmol, 2.5 eq) and Pd(dppf)Cl₂ (1.20 g, 1.63 mmol, 0.1 eq) under N₂ atmosphere. The mixture was stirred at 90° C. for 8 h under N₂ atmosphere. The residue was diluted with water (50 mL) and extracted with EtOAc (60 mL*3). The combined organic layers were washed with brine (80 mL*2), dried over Na₂SO₄, filtered and concentrated under reduced pressure to give a residue. The residue was purified by silica gel column chromatography (petroleum ether/ethyl acetate=10/1 to 1/1). Compound tert-butyl 4-(6-bromo-1,3-dimethyl-2-oxo-benzimidazol-4-yl)-3,6-dihydro-2H-pyridine-1-carboxylate (1.90 g, 4.50 mmol, 41% yield) was obtained as a yellow oil.

LC-MS: MS (ESI⁺: t_{R}=0.913 min, m/z=422.1 [M+H⁺]

To a mixture of tert-butyl 4-(6-bromo-1,3-dimethyl-2-oxo-benzimidazol-4-yl)-3,6-dihydro-2H-pyridine-1-carboxylate (800 mg, 1.89 mmol, 1.0 eq) in dioxane (8 mL) and H₂O (8 mL) was added KOH (318 mg, 5.68 mmol, 3.0 eq), Pd₂(dba)₃ (173 mg, 189 μmol, 0.1 eq) and tBuXphos (160 mg, 378 μmol, 0.2 eq) under N₂. The mixture was stirred at 90° C. for 12 h. The mixture was adjusted to pH about 4 with HCl (1 M), and the mixture was extracted with EtOAc (10 mL×3). The combined organic layers were washed with brine (10 mL*3), dried over Na₂SO₄, filtered and concentrated under reduced pressure to give a residue. The residue was purified by silica gel column chromatography (petroleum ether/ethyl acetate=10/1 to 1/3). Compound tert-butyl 4-(6-hydroxy-1,3-dimethyl-2-oxo-benzimidazol-4-yl)-3,6-dihydro-2H-pyridine-1-carboxylate (201 mg, 559 mol, 29% yield) was obtained as a yellow solid.

LC-MS: MS (ESI: t_{R}=0.562 min, m/z=360.1 [M+H⁺]

To a mixture of tert-butyl 4-(6-hydroxy-1,3-dimethyl-2-oxo-benzimidazol-4-yl)-3,6-dihydro-2H-pyridine-1-carboxylate (200 mg, 556 μmol, 1.0 eq) in MeOH (10 mL) was added Pd/C (100 mg, 10% purity) under N₂. The suspension was degassed under vacuum and purged with H₂ for three times. The mixture was stirred under H₂ (50 psi) at 30° C. for 12 h. The reaction mixture was filtered and washed with MeOH (50 mL×3). The collected filtrate was concentrated to give a crude product. Compound tert-butyl 4-(6-hydroxy-1,3-dimethyl-2-oxo-benzimidazol-4-yl)piperidine-1-carboxylate (140 mg) was obtained as a red solid and directly used into the next step without further purification.

LC-MS: MS (ESI: t_{R}=0.764 min, m/z=384.1 [M+Na⁺]

To a solution of tert-butyl 4-(6-hydroxy-1,3-dimethyl-2-oxo-benzimidazol-4-yl)piperidine-1-carboxylate (140 mg) in DCM (4 mL) was added Py (306 mg, 3.87 mmol, 312 μL, 10.0 eq) and Tf₂O (218 mg, 774 μmol, 127 μL, 2.0 eq) at 0° C. The mixture was stirred at 0° C. for 1 h. The reaction mixture was diluted with water (15 mL), adjusted to pH about 8 by sat. NaHCO₃, extracted with DCM (20 mL*3). The combined organic layers were dried over Na₂SO₄, filtered and the filtrate was concentrated under reduced pressure to give a residue. The residue was purified by prep-TLC (SiO₂, Petroleum ether/Ethyl acetate=1/3). Compound tert-butyl 4-[1,3-dimethyl-2-oxo-6-(trifluoromethylsulfonyloxy)benzimidazol-4-yl]piperidine-1-carboxylate (140 mg, 283 mol, 73% yield over two steps) was obtained as a white solid.

LC-MS: MS (ESI⁺): t_{R}=0.900 min, m/z=516.3 [M+Na⁺]

To a solution of tert-butyl 4-[1,3-dimethyl-2-oxo-6-(trifluoromethylsulfonyloxy)benzimidazol-4-yl]piperidine-1-carboxylate (140 mg, 283 μmol, 1.0 eq) and 3-(1-methylpyrazol-4-yl)-8-(4,4,5,5-tetramethyl-1,3,2-dioxaborolan-2-yl)isoquinoline (114 mg, 340 μmol, 1.2 eq) in dioxane (4 mL) and H₂O (0.8 mL) was added Pd(dppf)Cl₂ (20 mg, 28 μmol, 0.1 eq) and Na₂CO₃ (90 mg, 851 μmol, 3.0 eq). The mixture was stirred at 90° C. for 12 h under N₂. The reaction mixture was diluted with water (15 mL) and extracted with EtOAc (20 mL*3). The combined organic layers were washed with brine (30 mL), dried over Na₂SO₄, filtered and concentrated under reduced pressure to give a residue. The residue was purified by prep-TLC (SiO₂, Ethyl acetate/MeOH=10/1). Compound tert-butyl 4-[1,3-dimethyl-6-[3-(1-methylpyrazol-4-yl)-8-isoquinolyl]-2-oxo-benzimidazol-4-yl]piperidine-1-carboxylate (70 mg, 126 mol, 44% yield) was obtained as a yellow solid.

LC-MS: MS (ESI⁺): t_{R}=0.790 min, m/z=553.4 [M+H⁺]

To a solution of tert-butyl 4-[1,3-dimethyl-6-[3-(1-methylpyrazol-4-yl)-8-isoquinolyl]-2-oxo-benzimidazol-4-yl]piperidine-1-carboxylate (70 mg, 126 μmol, 1.0 eq) in DCM (3 mL) was added TFA (1.54 g, 13.4 mmol, 1 mL, 106.2 eq). The mixture was stirred at 25° C. for 1 h. The mixture was concentrated to give a crude product. Compound 1,3-dimethyl-6-[3-(1-methylpyrazol-4-yl)-8-isoquinolyl]-4-(4-piperidyl)benzimidazol-2-one (57 mg) was obtained as a yellow solid and directly used into the next step without further purification.

LC-MS: MS (ESI⁺): t_{R}=0.698 min, m/z=453.3 [M+H⁺]

To a solution of 1,3-dimethyl-6-[3-(1-methylpyrazol-4-yl)-8-isoquinolyl]-4-(4-piperidyl)benzimidazol-2-one (57 mg) and N-[3-(3-chloro-4-cyano-phenoxy)-2,2,4,4-tetramethyl-cyclobutyl]-2-(4-formyl-1-piperidyl)pyrimidine-5-carboxamide (68 mg, 138 μmol, 1.1 eq) in DCM (3 mL) was added Et₃N (63 mg, 629 μmol, 87 μL, 5.0 eq) and NaBH(OAc)₃ (80 mg, 377 μmol, 3.0 eq). The mixture was stirred at 25° C. for 12 h. The mixture was concentrated to give a residue. The residue was purified by prep-HPLC (column: Waters xbridge 150*25 mm 10 um; mobile phase: [water (NH₄HCO₃)-ACN]; gradient: 75%-95% B over 8 min). Compound N-[3-(3-chloro-4-cyano-phenoxy)-2,2,4,4-tetramethyl-cyclobutyl]-2-[4-[[4-[1,3-dimethyl-6-[3-(1-methylpyrazol-4-yl)-8-isoquinolyl]-2-oxo-benzimidazol-4-yl]-1-piperidyl]methyl]-1-piperidyl]pyrimidine-5-carboxamide (65.82 mg, 67.57 μmol, 53% yield over two steps) was obtained as a white solid.

¹H NMR (400 MHz, DMSO-d₆) δ=9.22 (s, 1H), 8.76-8.69 (m, 2H), 8.34 (s, 1H), 8.15-8.05 (m, 2H), 7.93-7.84 (m, 2H), 7.81-7.74 (m, 1H), 7.71-7.64 (m, 1H), 7.54-7.46 (m, 1H), 7.28-7.23 (m, 1H), 7.23-7.18 (m, 1H), 7.16-7.11 (m, 1H), 7.04-6.95 (m, 1H), 4.78-4.60 (m, 2H), 4.27 (s, 1H), 4.06-3.96 (m, 1H), 3.91 (s, 3H), 3.64 (s, 3H), 3.37 (s, 3H), 3.30-3.20 (m, 1H), 3.01-2.83 (m, 4H), 2.22-2.12 (m, 2H), 2.11-1.99 (m, 2H), 1.90-1.70 (m, 7H), 1.20 (s, 6H), 1.10 (s, 6H), 1.05-0.92 (in, 2H).

LC-MS: MS (ESI⁺): t_{R}=1.819 min m/z=932.7 [M+H⁺]

The compounds below were prepared in a similar manner as described in Example 27.

| | | | LC-MS |
|---|---|---|---|
| Compound | Name | ¹H NMR (DMSO-d6) | [M + H⁺] |
| 243 | N-[3-(3-chloro-4-cyano- | δ = 9.24 (s, 1H), 8.79-8.70 (m, 2H), | 930.7 |
| | phenoxy)-2,2,4,4-tetramethyl- | 8.33 (s, 1H), 8.14 (s, 1H), 8.10 (d, J = | |
| | cyclobutyl]-2-[4-[[4-[1,3- | 15.2 Hz, 2H), 7.92-7.86 (m, 2H), 7.78 | |
| | dimethyl-6-[3-(1- | (s, J = 7.7 Hz, 1H), 7.72-7.67 (m, 1H), | |
| | methylpyrazol-4-yl)-8- | 7.52 (d, J = 6.4 Hz, 1H), 7.33 (d, J = 1.5 | |
| | isoquinolyl]-2-oxo- | Hz, 1H), 7.21 (d, J = 2.3 Hz, 1H), 7.03-6.97 | |
| | benzimidazol-4-yl]-3,6- | (m, 2H), 5.79 (s, 1H), 4.81-4.68 | |
| | dihydro-2H-pyridin-1- | (m, 2H), 4.28 (s, 1H), 4.03 (d, J = 9.2 Hz, | |
| | yl]methyl]-1- | 1H), 3.91 (s, 3H), 3.40 (d, J = 9.0 Hz, | |
| | piperidyl]pyrimidine-5- | 6H), 3.20-3.09 (m, 2H), 3.04-2.91 (m, | |
| | carboxamide | 2H), 2.75-2.63 (m, 2H), 2.39-2.25 (m, | |
| | | 2H), 2.01-1.89 (m, 1H), 1.88-1.78 (m, | |
| | | 2H), 1.21 (s, 7H), 1.16-0.98 (m, 9H). | |
| 214 | N-[3-(3-chloro-4-cyano- | δ = 8.73 (s, 2H), 7.96-7.85 (m, 1H), | 986.7 |
| | phenoxy)-2,2,4,4-tetramethyl- | 7.80-7.65 (m, 2H), 7.48 (s, 1H), 7.25-7.18 | |
| | cyclobutyl]-2-[4-[[4-[6-[7- | (m, 1H), 7.09 (s, 1H), 7.05-6.96 | |
| | (difluoromethyl)-6-(1- | (m, 2H), 6.95-6.55 (m, 3H), 4.83-4.63 | |
| | methylpyrazol-4-yl)-3,4- | (m, 2H), 4.28 (s, 1H), 4.09-3.96 (m, | |
| | dihydro-2H-quinolin-1-yl]-1,3- | 1H), 3.86 (s, 3H), 3.72-3.51 (m, 5H), | |
| | dimethyl-2-oxo-benzimidazol- | 3.30 (s, 3H), 3.26-3.13 (m, 1H), 3.07-2.79 | |
| | 4-yl]-1-piperidyl]methyl]-1- | (m, 6H), 2.28-1.95 (m, 6H), 1.93-1.60 | |
| | piperidyl]pyrimidine-5- | (m, 7H), 1.21 (s, 6H), 1.10 (s, 6H), | |
| | carboxamide | 1.05-0.94 (m, 2H). | |
| 242 | N-[3-(3-chloro-4-cyano- | δ = 8.74 (s, 2H), 8.00-7.85 (m, 1H), | 984.7 |
| | phenoxy)-2,2,4,4-tetramethyl- | 7.78-7.65 (m, 2H), 7.53-7.43 (m, 1H), | |
| | cyclobutyl]-2-[4-[[4-[6-[7- | 7.25-7.17 (m, 1H), 7.16-7.04 (m, 2H), | |
| | (difluoromethyl)-6-(1- | 7.04-6.96 (m, 1H), 6.92-6.56 (m, 3H), | |
| | methylpyrazol-4-yl)-3,4- | 5.68 (s, 1H), 4.85-4.62 (m, 2H), 4.29 (s, | |
| | dihydro-2H-quinolin-1-yl]-1,3- | 1H), 4.13-3.97 (m, 1H), 3.86 (s, 3H), | |
| | dimethyl-2-oxo-benzimidazol- | 3.67-3.53 (m, 2H), 3.35 (s, 3H), 3.31 (s, | |
| | 4-yl]-3,6-dihydro-2H-pyridin-1- | 3H), 3.13-3.03 (m, 2H), 3.02-2.93 (m, | |
| | yl] methyl]-1-piperidyl] | 2H), 2.86 (s, 2H), 2.74-2.58 (m, 3H), | |
| | pyrimidine-5-carboxamide | 2.41-2.35 (m, 2H), 2.31-2.22 (m, 2H), | |
| | | 2.04-1.97 (m, 2H), 1.88-1.77 (m, 2H), | |
| | | 1.21 (s, 6H), 1.11 (s, 6H), 1.08-1.00 (m, | |
| | | 2H). | |

### Example 28: Synthesis of 1-[4-[[4-[5-acetyl-3-[7-(difluoromethyl)-6-(1-methylpyrazol-3-yl)-3,4-dihydro-2H-quinolin-1-yl]-6,7-dihydro-4H-pyrazolo[4,3-c]pyridin-1-yl]-1-piperidyl]methyl]cyclohexyl]-N—[3-(3-chloro-4-cyano-phenoxy)-2,2,4,4-tetramethyl-cyclobutyl]pyrazole-4-carboxamide (Compound 241)

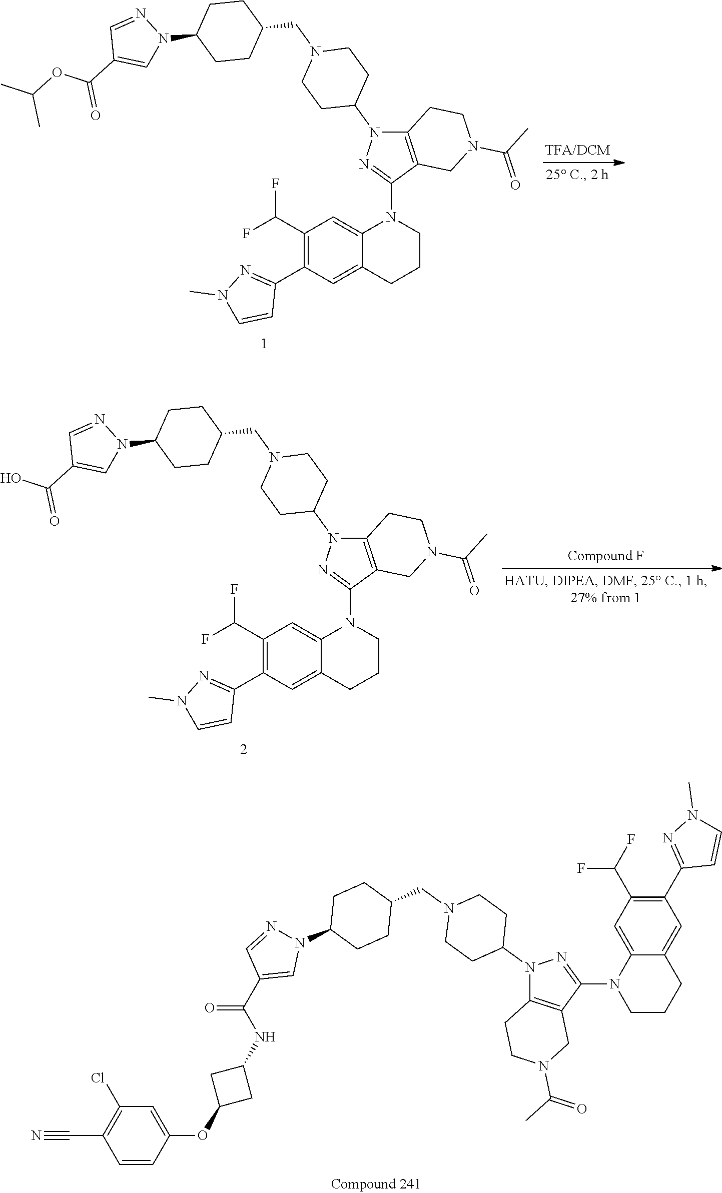 [SMILES: CC(=O)N1CCc2c(c(N3CCCc4cc(-c5ccn(C)n5)c(C(F)F)cc43)nn2C2CCN(C[C@H]3CC[C@H](n4cc(C(=O)OC(C)C)cn4)CC3)CC2)C1 ; CC(=O)N1CCc2c(c(N3CCCc4cc(-c5ccn(C)n5)c(C(F)F)cc43)nn2C2CCN(C[C@H]3CC[C@H](n4cc(C(=O)O)cn4)CC3)CC2)C1 ; CC(=O)N1CCc2c(c(N3CCCc4cc(-c5ccn(C)n5)c(C(F)F)cc43)nn2C2CCN(C[C@H]3CC[C@H](n4cc(C(=O)N[C@H]5C[C@H](Oc6ccc(C#N)c(Cl)c6)C5)cn4)CC3)CC2)C1]

Synthesis of 2A was reported in Journal of Medicinal Chemistry, 2019, vol. 62, #24, p. 11218-11231.

### Synthesis of 1:

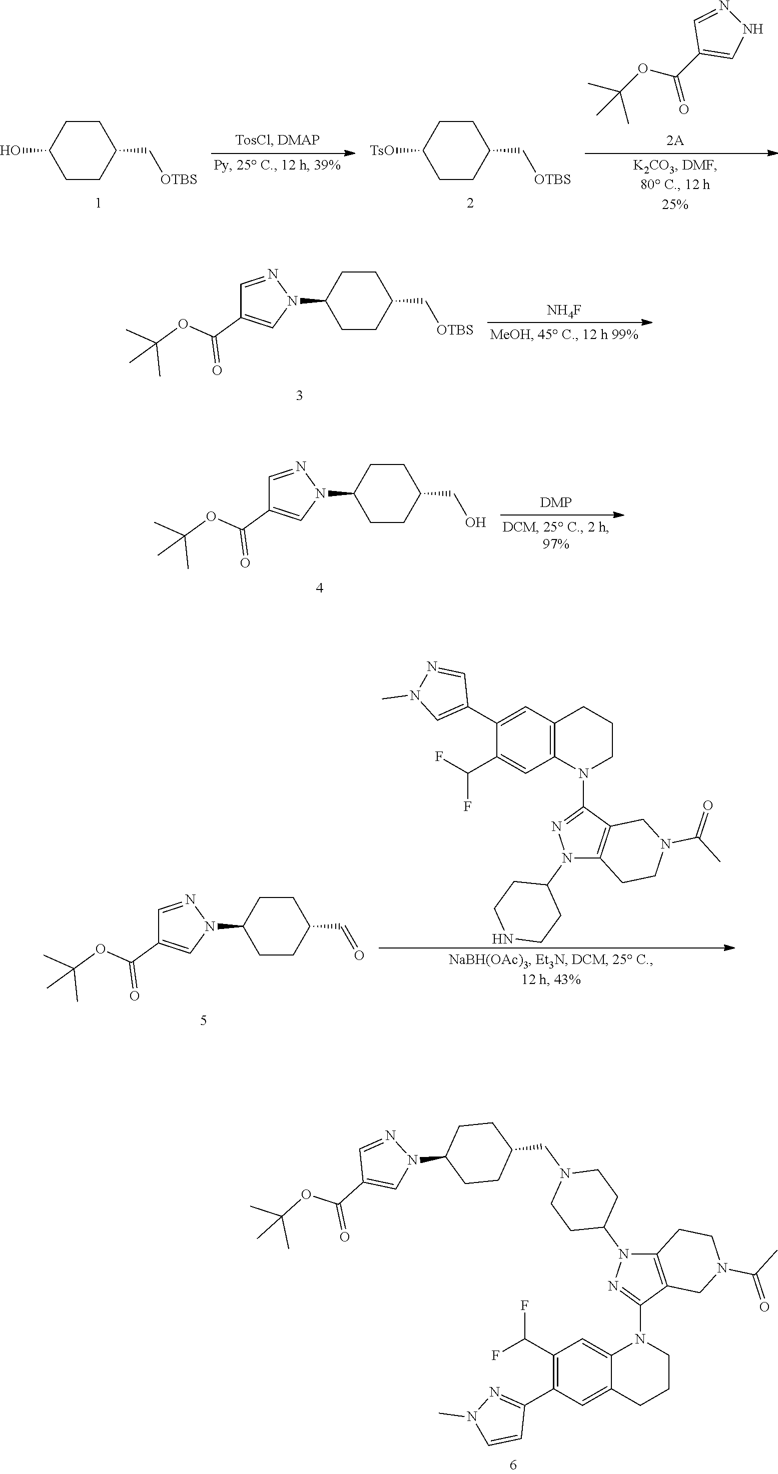

To a mixture of 4-[[tert-butyl(dimethyl)silyl]oxymethyl]cyclohexanol (2.80 g, 11.5 mmol, 1.0 eq) in Py (50 mL) was added DMAP (280 mg, 2.30 mmol, 0.2 eq) and TosCl (2.80 g, 14.7 mmol, 1.3 eq). The mixture was stirred at 25° C. for 12 h under N₂ atmosphere. The reaction mixture was quenched by addition water 100 mL, and then extracted with ethyl acetate (30 mL×3). The combined organic layers were washed with brine (30 mL), dried over Na₂SO₄, filtered and concentrated under reduced pressure to give a residue. The residue was purified by column chromatography (SiO₂, Petroleum ether: Ethyl acetate=1:0 to 20:1). Compound [4-[[tert-butyl(dimethyl)silyl]oxymethyl]cyclohexyl]4-methylbenzenesulfonate (1.80 g, 4.52 mmol, 39% yield) was obtained as a Colorless oil.

¹H NMR (400 MHz, CHLOROFORM-d) δ ppm 7.80 (d, J=8.4 Hz, 2H) 7.33 (d, J=8.0 Hz, 2H) 4.76 (s, 1H) 3.40 (d, J=6.4 Hz, 2H) 2.45 (s, 3H) 1.88 (m, 2H) 1.41-1.61 (m, 5H) 1.17-1.41 (m, 2H) 0.88 (s, 9H) 0.03 (s, 6H).

A mixture of tert-butyl 1H-pyrazole-4-carboxylate (900 mg, 5.35 mmol, 1.2 eq), [4-[[tert-butyl(dimethyl)silyl]oxymethyl]cyclohexyl]4-methylbenzenesulfonate (1.80 g, 4.52 mmol, 1.0 eq), K₂CO₃ (1.80 g, 13.0 mmol, 2.9 eq) in DMF (40 mL) was degassed and purged with N₂ for 3 times, and then the mixture was stirred at 80° C. for 12 h under N₂ atmosphere. The reaction mixture was quenched by addition water 250 mL, and then extracted with ethyl acetate (30 mL×3). The combined organic layers were washed with brine (30 mL×3), dried over Na₂SO₄, filtered and concentrated under reduced pressure to give a residue. The residue was purified by column chromatography (SiO₂, Petroleum ether: Ethyl acetate=1:0 to 5:1). Compound tert-butyl 1-[4-[[tert-butyl(dimethyl)silyl]oxymethyl]cyclohexyl]pyrazole-4-carboxylate (450 mg, 1.14 mmol, 25% yield) was obtained as a colorless oil.

LC-MS: MS (ESI⁺): t_{R}=0.768 min, m/z=395.2 [M+H⁺]

A mixture of tert-butyl 1-[4-[[tert-butyl(dimethyl)silyl]oxymethyl]cyclohexyl]pyrazole-4-carboxylate (400 mg, 1.01 mmol, 1.0 eq), NH₄F (400 mg, 10.8 mmol, 10.7 eq) in MeOH (20 mL) was degassed and purged with N₂ for 3 times, and then the mixture was stirred at 45° C. for 12 h under N₂ atmosphere. Afterwards, the reaction mixture was concentrated under reduced pressure to remove solvent. The residue was purified by column chromatography (SiO₂, Dichloromethane:Methanol=30:1 to 10:1). Compound tert-butyl 1-[4-(hydroxymethyl)cyclohexyl]pyrazole-4-carboxylate (280 mg, 999 μmol, 99% yield) was obtained as a yellow solid.

LC-MS: MS (ESI⁺): t_{R}=0.527 min, m/z=281.2 [M+H⁺]

A mixture of tert-butyl 1-[4-(hydroxymethyl)cyclohexyl]pyrazole-4-carboxylate (280 mg, 999 μmol, 1.0 eq), DMP (560 mg, 1.32 mmol, 1.3 eq) in DCM (10 mL) was degassed and purged with N2 for 3 times, and then the mixture was stirred at 25° C. for 2 h under N2 atmosphere. Afterwards, the reaction mixture was quenched by addition saturated eq. NaHCO3 (50 mL) and saturated eq. Na₂SO₃ (50 mL) at 0° C., and then extracted with DCM (40 mL×3). The combined organic layers were washed with brine (40 mL), dried over Na2SO4, filtered and concentrated under reduced pressure to give a residue. The residue was purified by column chromatography (SiO2, Petroleum ether: Ethyl acetate=1:0 to 0:1). Compound tert-butyl 1-(4-formylcyclohexyl)pyrazole-4-carboxylate (270 mg, 970 μmol, 97% yield) was obtained as a yellow solid.

LC-MS: MS (ESI⁺): t_{R}=0.552 min, m/z=279.2 [M+H⁺]

To a solution of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (100 mg, 196 μmol, 1.0 eq), tert-butyl 4-formylpiperidine-1-carboxylate (50 mg, 234 μmol, 1.2 eq) and Et₃N (218 mg, 2.16 mmol, 11.0 eq) in DCM (5 mL) was added NaBH(OAc)₃ (210 mg, 991 μmol, 5.1 eq). The mixture was stirred at 25° C. for 12 h, and then the mixture was diluted with DCM (100 mL), washed with water (15 mL×2), brine (15 mL), dried with anhydrous sodium sulfate, filtered and concentrated under vacuum. The residue was purified by prep-TLC (SiO₂, ethyl acetate:MeOH=10:1). Compound tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]piperidine-1-carboxylate (61 mg, 84.9 μmol, 43% yield) was obtained as a yellow oil.

LC-MS: MS (ESI⁺): t_{R}=0.527 min, m/z=772.5 [M+H⁺]

### Synthesis of Compound 241:

A mixture of tert-butyl 1-[4-[[4-[5-acetyl-3-[7-(difluoromethyl)-6-(1-methylpyrazol-3-yl)-3,4-dihydro-2H-quinolin-1-yl]-6,7-dihydro-4H-pyrazolo[4,3-c]pyridin-1-yl]-1-piperidyl]methyl]cyclohexyl]pyrazole-4-carboxylate (80 mg, 104 μmol, 1.0 eq), TFA (480 mg, 4.00 mmol, 40.0 eq) in DCM (10 mL) was stirred at 25° C. for 2 h. The reaction mixture was concentrated under reduced pressure to give a residue. Compound 1-[4-[[4-[5-acetyl-3-[7-(difluoromethyl)-6-(1-methylpyrazol-3-yl)-3,4-dihydro-2H-quinolin-1-yl]-6,7-dihydro-4H-pyrazolo[4,3-c]pyridin-1-yl]-1-piperidyl]methyl]cyclohexyl]pyrazole-4-carboxylic acid (85 mg) was obtained as a white solid and directly used in the next step without further purification. LC-MS: MS (ESI⁺): t_{R}=0.452 min, m/z=716.4 [M+H⁺]

To a solution of 1-[4-[[4-[5-acetyl-3-[7-(difluoromethyl)-6-(1-methylpyrazol-3-yl)-3,4-dihydro-2H-quinolin-1-yl]-6,7-dihydro-4H-pyrazolo[4,3-c]pyridin-1-yl]-1-piperidyl]methyl]cyclohexyl]pyrazole-4-carboxylic acid (84 mg, 101 mol, 1.0 eq) and 4-(3-amino-2,2,4,4-tetramethyl-cyclobutoxy)-2-chloro-benzonitrile (50 mg, 179 μmol, 1.8 eq) in DMF (4 mL) was added DIPEA (110 mg, 849 μmol, 8.4 eq) and HATU (59 mg, 155 μmol, 1.5 eq) and stirred at 25° C. for 1 h. The mixture was diluted with ethyl acetate (100 mL) washed with water (15 mL×2), brine (15 mL), dried with anhydrous sodium sulfate, filtered and concentrated under vacuum. The crude product was purified by reversed-phase HPLC (column: Phenomenex luna C18 150×25 mm×10 um; mobile phase: [water (FA)-ACN]; gradient: 30%-60% B over 10 min). Compound 1-[4-[[4-[5-acetyl-3-[7-(difluoromethyl)-6-(1-methylpyrazol-3-yl)-3,4-dihydro-2H-quinolin-1-yl]-6,7-dihydro-4H-pyrazolo[4,3-c]pyridin-1-yl]-1-piperidyl]methyl]cyclohexyl]-N-[3-(3-chloro-4-cyano-phenoxy)-2,2,4,4-tetramethyl-cyclobutyl]pyrazole-4-carboxamide (28.5 mg, 27.4 μmol, 27% yield over two steps) was obtained as a yellow solid.

¹H NMR (400 MHz, CHLOROFORM-d) δ ppm 8.37 (s, 1H) 7.91 (s, 1H) 7.72 (s, 1H) 7.50-7.62 (m, 2H) 7.41 (d, J=6.4 Hz, 1H) 7.03 (d, J=20.0 Hz, 1H) 6.97 (d, J=2.4 Hz, 1H) 6.89 (d, J=11.2 Hz, 1H) 6.81 (m, 1H) 6.33-6.73 (m, 1H) 5.81 (d, J=8.0 Hz, 1H) 4.28 (s, 1H) 4.07-4.16 (m, 3H) 3.93-4.06 (m, 5H) 3.91 (m, 1H) 3.63-3.80 (m, 3H) 3.02-3.23 (m, 2H) 2.78-2.94 (m, 1H) 2.68-2.77 (m, 1H) 2.67-2.78 (m, 1H) 2.65-2.79 (m, 1H) 2.20-2.41 (m, 8H) 1.99-2.13 (m, 12H) 1.68-1.83 (m, 3H) 1.18-1.29 (m, 12H) 1.09-1.18 (m, 2H)

LC-MS: MS (ESI⁺): t_{R}=1.981 min, m/z=976.8 [M+H⁺]

### Example 29: Synthesis of 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.5]nonan-2-yl]-N-[3-(4-cyano-3-methoxy-phenyl)-2,2,4,4-tetramethyl-cyclobutyl]benzamide (Compound 273)

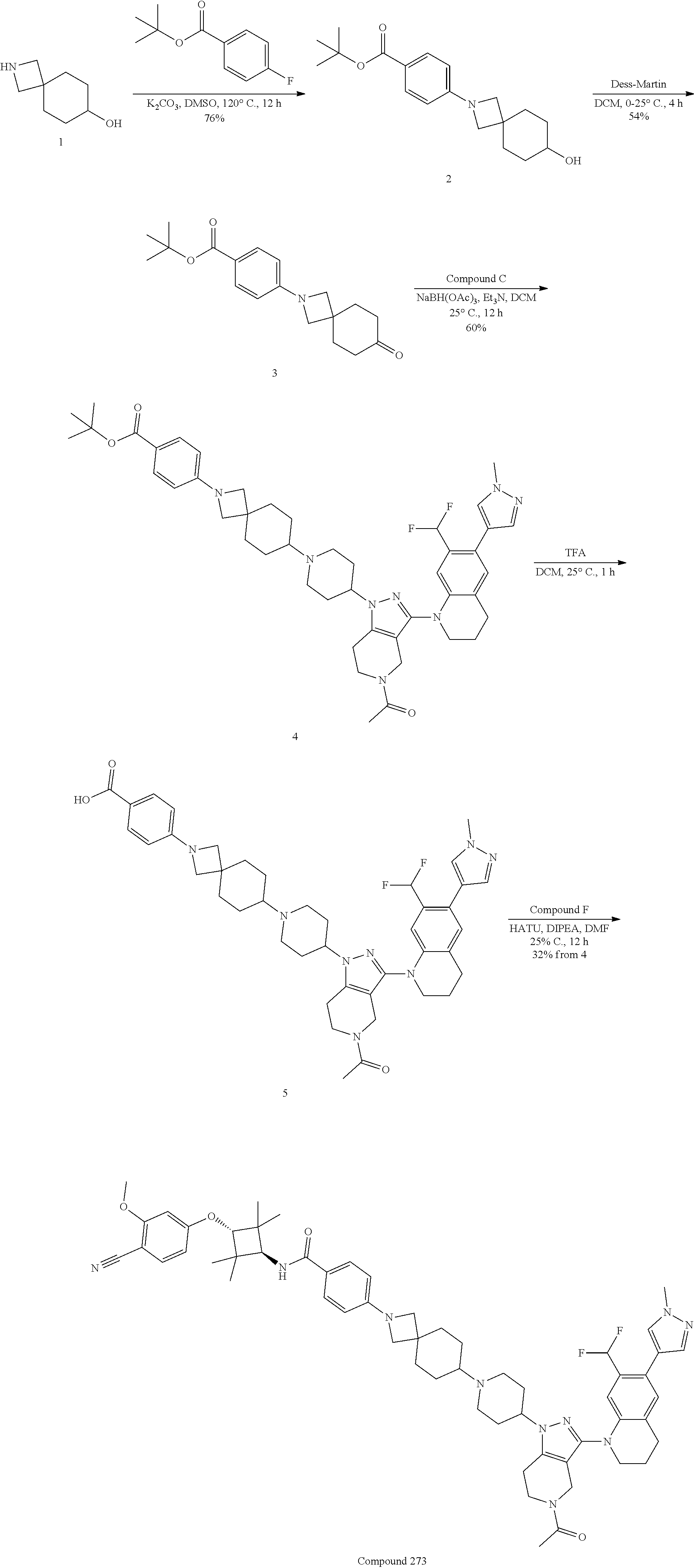

Synthesis of 1 was reported in WO2023/278402 A1

To a mixture of 2-azaspiro[3.5]nonan-7-ol (2.89 g, 20.4 mmol, 1.0 eq) and tert-butyl 4-fluorobenzoate (8.03 g, 40.9 mmol, 2.0 eq) in DMSO (30 mL) was added K₂CO₃ (14.14 g, 102.33 mmol, 5.0 eq), the mixture was stirred at 120° C. for 12 h. The reaction mixture was diluted with water (100 mL) and extracted with EtOAc (50 mL*3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=10/1 to 1/1). Compound tert-butyl 4-(7-hydroxy-2-azaspiro[3.5]nonan-2-yl)benzoate (5.00 g, 15.75 mmol, 76% yield) was obtained as a white solid.

LC-MS: MS (ESI⁺: t_{R}=0.842 min, m/z=318.4 [M+H⁺]

To a mixture of tert-butyl 4-(7-hydroxy-2-azaspiro[3.5]nonan-2-yl)benzoate (4.80 g, 15.12 mmol, 1.0 eq) in DCM (400 mL) was added Dess-Martin (12.83 g, 30.24 mmol, 9.3 mL, 2.0 eq) at 0° C., the mixture was stirred at 25° C. for 4 h. The reaction mixture was diluted with water (200 mL), extracted with DCM (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=20/1 to 2/1). Compound tert-butyl 4-(7-oxo-2-azaspiro[3.5]nonan-2-yl)benzoate (2.60 g, 8.24 mmol, 54% yield) was obtained as a white solid.

¹H NMR (400 MHz, CDCl₃) δ=7.86 (d, J=8.8 Hz, 2H), 6.39 (d, J=8.8 Hz, 2H), 3.81 (s, 4H), 2.46-2.34 (m, 4H), 2.20-2.11 (m, 4H), 1.57 (s, 9H).

LC-MS: MS (ESI⁺: t_{R}=0.848 min, m/z=316.5 [M+H⁺]

To a mixture of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (1.50 g, 2.94 mmol, 1.0 eq) and tert-butyl 4-(7-oxo-2-azaspiro[3.5]nonan-2-yl)benzoate (1.39 g, 4.42 mmol, 1.5 eq) in DCM (30 mL) was added Et₃N (1.49 g, 14.72 mmol, 2.1 mL, 5.0 eq) and NaBH(OAc)₃ (1.87 g, 8.83 mmol, 3.0 eq), the mixture was stirred at 25° C. for 12 h. The reaction mixture was diluted with water (30 mL), extracted with DCM (30 mL*3). The combined organic layers were dried over Na₂SO₄, filtered and the filtrate was concentrated under reduced pressure to give a residue. The residue was purified by column chromatography (SiO₂, DCM/MeOH=100/1 to 10/1, NH₃·H₂O). Compound tert-butyl 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.5]nonan-2-yl]benzoate (1.45 g, 1.79 mmol, 60% yield) was obtained as a light yellow solid.

¹H NMR (400 MHz, CDCl₃) δ=7.88-7.79 (m, 2H), 7.58-7.49 (m, 1H), 7.44-7.37 (m, 1H), 7.08-6.94 (m, 1H), 6.86 (s, 1H), 6.72-6.27 (m, 3H), 4.26-4.10 (m, 3H), 3.97-3.93 (m, 3H), 3.92-3.86 (m, 2H), 3.79-3.67 (m, 3H), 3.65 (s, 2H), 3.60 (s, 2H), 3.15-3.00 (m, 2H), 2.92-2.70 (m, 4H), 2.36-2.28 (m, 2H), 2.28-2.18 (m, 2H), 2.11-1.99 (m, 8H), 1.98-1.90 (m, 2H), 1.89-1.79 (m, 3H), 1.59-1.55 (m, 9H), 1.42-1.30 (m, 2H).

LC-MS: MS (ESI⁺: t_{R}=0.810 min, m/z=809.3 [M+H⁺]

To a mixture of tert-butyl 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.5]nonan-2-yl]benzoate (1.45 g, 1.79 mmol, 1.0 eq) in DCM (10 mL) was added TFA (7.68 g, 67.31 mmol, 5 mL, 37.5 eq), the mixture was stirred at 25° C. for 1 h. The mixture was concentrated to give a crude product. Compound 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.5]nonan-2-yl]benzoic acid (1.35 g) was obtained as a yellow solid and directly used into the next step without further purification.

LC-MS: MS (ESI⁺): t_{R}=0.728 min, m/z=753.3 [M+H⁺]

To a mixture of 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.5]nonan-2-yl]benzoic acid (1.35 g) and 4-(3-amino-2,2,4,4-tetramethyl-cyclobutoxy)-2-methoxy-benzonitrile (585 mg, 1.88 mmol, 1.05 eq) in DMF (15 mL) was added DIPEA (1.16 g, 8.97 mmol, 1.5 mL, 5.0 eq) and HATU (818 mg, 2.15 mmol, 1.2 eq), the mixture was stirred at 25° C. for 12 h. The reaction mixture was diluted with water (60 mL) and extracted with EtOAc (50 mL*3). The combined organic layers were washed with brine (100 mL), dried over Na₂SO₄, filtered and concentrated under reduced pressure to give a residue. The residue was purified by column chromatography (SiO₂, DCM/MeOH=100/1 to 10/1, NH₃H₂O) to give a crude product, then the crude product was purified by prep-HPLC (column: Phenomenex luna C18 150*40 mm*15 um; mobile phase: [water (FA)-ACN]; gradient: 40%-70% B over 10 min). Compound 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.5]nonan-2-yl]-N-[3-(4-cyano-3-methoxy-phenoxy)-2,2,4,4-tetramethyl-cyclobutyl]benzamide (584.55 mg, 575.61 μmol, 32% yield over two steps) was obtained as a yellow solid.

¹H NMR (400 MHz, DMSO-d₆) δ=8.17 (s, 1H), 7.86-7.69 (m, 3H), 7.64 (d, J=8.8 Hz, 1H), 7.50 (s, 1H), 7.46-7.38 (m, 1H), 7.10 (s, 1H), 6.97-6.61 (m, 3H), 6.58-6.50 (m, 1H), 6.48-6.35 (m, 2H), 4.26 (s, 1H), 4.18-4.10 (m, 2H), 4.08-4.01 (m, 2H), 3.90 (s, 3H), 3.86 (s, 3H), 3.76-3.66 (m, 3H), 3.64-3.50 (m, 7H), 3.05-2.95 (m, 2H), 2.88-2.70 (m, 4H), 2.44-2.38 (m, 2H), 2.07 (s, 2H), 2.02-1.85 (m, 8H), 1.80-1.68 (m, 2H), 1.58-1.46 (m, 2H), 1.44-1.32 (m, 2H), 1.32-1.02 (m, 12H).

LC-MS: MS (ESI⁺): t_{R}=2.085 min, m/z=1009.4 [M+H⁺]

The compounds below were prepared in a similar manner as described in Example 29.

| | | | LC-MS |
|---|---|---|---|
| Compound | Name | ¹H NMR (CDCl₃) | [M + H⁺] |
| 262 | 4-[2-[4-[5-acetyl-3-[7- | δ = 7.68 (d, J = 8.8 Hz, 2H), 7.54 (d, J = | 1009.6 |
| | (difluoromethyl)-6-(1- | 5.2 Hz, 1H), 7.48-7.38 (m, 2H), 7.04 (d, | |
| | methylpyrazol-4-yl)-3,4- | J = 18.0 Hz, 1H), 6.96-6.83 (m, 3H), | |
| | dihydro-2H-quinolin-1-yl]-6,7- | 6.71-6.38 (m, 3H), 6.12 (d, J = 8.2 Hz, | |
| | dihydro-4H-pyrazolo[4,3- | 1H), 4.29 (s, 1H), 4.16-4.12 (m, 2H), | |
| | c]pyridin-1-yl]-1-piperidyl]-7- | 4.05 (s, 1H), 3.96 (s, 3H), 3.94-3.88 (m, | |
| | azaspiro[3.5]nonan-7-yl]-N-[3- | 4H), 3.80-3.63 (m, 3H), 3.30 (s, 2H), | |
| | (4-cyano-3-methoxy-phenoxy)- | 3.25-3.07 (m, 4H), 3.03-2.79 (m, 4H), | |
| | 2,2,4,4-tetramethyl- | 2.74 (t, J = 5.6 Hz, 1H), 2.32-2.21 (m, | |
| | cyclobutyl]benzamide | 3H), 2.17 (s, 2H), 2.14-2.03 (m, 7H), | |
| | | 1.93-1.76 (m, 8H), 1.25 (d, J = 10.4 Hz, | |
| | | 12H) | |
| 274 | 2-[7-[4-[5-acetyl-3-[7- | δ = 11.07-11.04 (m, 1 H), 8.69 (s, 2 H), | 1015.6 |
| | (difluoromethyl)-6-(1- | 7.60-7.50 (m, 2 H), 7.42-7.36 (m, 1 H), | |
| | methylpyrazol-4-yl)-3,4- | 7.06-6.93 (m, 2 H), 6.89-6.74 (m, | |
| | dihydro-2H-quinolin-1-yl]-6,7- | 2 H), 6.71-6.34 (m, 1 H), 5.94-5.88 (m, | |
| | dihydro-4H-pyrazolo[4,3- | 1 H), 4.24 (s, 1 H), 4.14-4.09 (m, 2 H), | |
| | c]pyridin-1-yl]-1-piperidyl]-2- | 4.04 (s, 1 H), 3.95 (s, 3 H), 3.91-3.83 | |
| | azaspiro[3.5]nonan-2-yl]-N-[3- | (m, 5 H), 3.78-3.64 (m, 3 H), 3.23-2.93 | |
| | (3-chloro-4-cyano-phenoxy)- | (m, 2 H), 2.92-2.77 (m, 3 H), 2.77-2.71 | |
| | 2,2,4,4-tetramethyl- | (m, 1 H), 2.50-2.11 (m, 6 H), | |
| | cyclobutyl]pyrimidine-5- | 2.11-2.00 (m, 6 H), 1.99-1.76 (m, | |
| | carboxamide | 4 H), 1.60-1.49 (m, 3 H), 1.44-1.30 (m, | |
| | | 2 H), 1.23 (d, J = 12.0 Hz, 12 H). | |
| 300 | 4-[4-[9-[5-acetyl-3-[7- | δ = 7.70-7.68 (m, 2 H), 7.53-7.52 (d, | 1037.7 |
| | (difluoromethyl)-6-(1- | J = 4.4 Hz, 1 H), 7.47-7.45 (d, J = 8.4 Hz, | |
| | methylpyrazol-4-yl)-3,4- | 1 H), 7.41-7.40 (d, J = 6.0 Hz, 1 H), 7.04-7.02 | |
| | dihydro-2H-quinolin-1-yl]-6,7- | (m, 1 H), 6.92-6.90 (m, 3 H), 6.70-6.41 | |
| | dihydro-4H-pyrazolo[4,3- | (m, 3 H), 6.13 (d, J = 8.8 Hz, 1 H), | |
| | c]pyridin-1-yl]-3- | 4.27 (s, 1 H), 4.16-4.13 (m, 3H), 3.95-3.94 | |
| | azaspiro[5.5]undecan-3-yl]-1- | (m, 4 H), 3.92-3.90 (m, 6H), 3.70-3.68 | |
| | piperidyl]-N-[3-(4-cyano-3- | (m, 3 H), 2.90-2.85 (m, 5 H), 2.85-2.75 | |
| | methoxy-phenoxy)-2,2,4,4- | (m, 4H), 2.74-2.60 (m, 1 H), 2.25-2.22 | |
| | tetramethyl- | (m, 2 H), 2.20-2.10 (m, 7 H), 1.90-1.80 | |
| | cyclobutyl]benzamide | (m, 3 H), 1.80-1.70 (m, 4 H), 1.70-1.60 | |
| | | (m, 4 H), 1.40-1.30 (m, 2 H), 1.25 | |
| | | (d, J = 10.4 Hz, 12 H). | |
| 301 | 4-[3-[9-[5-acetyl-3-[7- | δ = 7.65 (d, J = 8.8 Hz, 2H), 7.53 (d, J = | 1009.7 |
| | (difluoromethyl)-6-(1- | 5.2 Hz, 1H), 7.45 (d, J = 8.8 Hz, 1H), | |
| | methylpyrazol-4-yl)-3,4- | 7.43-7.37 (m, 1H), 7.09-6.97 (m, 1H), | |
| | dihydro-2H-quinolin-1-yl]-6,7- | 6.94-6.83 (m, 1H), 6.68-6.37 (m, 5H), | |
| | dihydro-4H-pyrazolo[4,3- | 6.09 (d, J = 8.0 Hz, 1H), 4.28-4.12 (m, | |
| | c]pyridin-1-yl]-3- | 3H), 4.08-4.02 (m, 3H), 3.96 (d, J = | |
| | azaspiro[5.5]undecan-3- | 2.14 Hz, 3H), 3.93-3.86 (m, 5H), 3.80 | |
| | yl]azetidin-1-yl]-N-[3-(4- | (t, J = 6.8 Hz, 2H), 3.77-3.66 (m, 3H), | |
| | cyano-3-methoxy-phenoxy)- | 3.39-3.29 (m, 1H), 2.92-2.83 (m, 2H), | |
| | 2,2,4,4-tetramethyl- | 2.79 (t, J = 6.0 Hz, 1H), 2.73 (t, J = 5.6 | |
| | cyclobutyl]benzamide | Hz, 1H), 2.39 (s, 4H), 2.18-2.04 (m, | |
| | | 7H), 1.88 (d, J = 12.0 Hz, 2H), 1.82-1.73 | |
| | | (m, 6H), 1.49 (s, 2H), 1.25 (d, J = | |
| | | 11.2 Hz, 12H) | |
| 310 | 4-[3-[4-[5-acetyl-3-[7- | (CD₃OD) δ = 7.73 (d, J = 8.8 Hz, 2H), | 941.6 |
| | (difluoromethyl)-6-(1- | 7.66-7.61 (m, 1H), 7.55-7.49 (m, 2H), | |
| | methylpyrazol-4-yl)-3,4- | 7.45-7.07 (m, 2H), 6.76-6.69 (m, 1H), | |
| | dihydro-2H-quinolin-1-yl]-6,7- | 6.64-6.48 (m, 4H), 4.33-4.05 (m, 6H), | |
| | dihydro-4H-pyrazolo[4,3- | 3.99-3.86 (m, 7H), 3.85-3.73 (m, 3H), | |
| | c]pyridin-1-yl]-1- | 3.72-3.62 (m, 2H), 3.51-3.43 (m, 1H), | |
| | piperidyl]azetidin-1-yl]-N-[3- | 3.39-3.33 (m, 2H), 3.16-3.03 (m, 2H), | |
| | (4-cyano-3-methoxy-phenoxy)- | 2.99-2.78 (m, 4H), 2.26-2.13 (m, 5H), | |
| | 2,2,4,4-tetramethyl- | 2.13-1.95 (m, 5H), 1.35-1.26 (s, 6H), | |
| | cyclobutyl]benzamide | 1.25-1.20 (s, 6H) | |
| 333 | 2-[3-[4-[5-acetyl-3-[7- | δ = 8.72 (s, 2H), 7.65-7.52 (m, 2H), | 947.3 |
| | (difluoromethyl)-6-(1- | 7.45-7.39 (m, 1H), 7.10-6.94 (m, 2H), | |
| | methylpyrazol-4-yl)-3,4- | 6.90-6.78 (m, 2H), 6.74-6.32 (m, 1H), | |
| | dihydro-2H-quinolin-1-yl]-6,7- | 5.96 (s, 1H), 4.35-4.21 (m, 3H), 4.18-4.04 | |
| | dihydro-4H-pyrazolo[4,3- | (m, 5H), 4.03-3.89 (m, 5H), 3.81-3.65 | |
| | c]pyridin-1-yl]-1- | (m, 3H), 3.45-3.32 (m, 1H), 3.05 | |
| | piperidyl]azetidin-1-yl]-N-[3- | (s, 2H), 2.93-2.72 (m, 4H), 2.38-2.23 | |
| | (3-chloro-4-cyano-phenoxy)- | (m, 2H), 2.19-1.97 (m, 9H), 1.30-1.20 | |
| | 2,2,4,4-tetramethyl- | (m, 12H) | |
| | cyclobutyl]pyrimidine-5- | | |
| | carboxamide | | |
| 334 | 2-[(3S)-3-[4-[5-acetyl-3-[7- | δ = 8.74 (s, 2H), 7.61-7.51 (m, 2H), | 961.7 |
| | (difluoromethyl)-6-(1- | 7.43-7.38 (m, 1H), 7.08-6.95 (m, 2H), | |
| | methylpyrazol-4-yl)-3,4- | 6.90-6.77 (m, 2H), 6.70-6.34 (m, 1H), | |
| | dihydro-2H-quinolin-1-yl]-6,7- | 5.99-5.89 (m, 1H), 4.25 (s, 1H), 4.17-4.09 | |
| | dihydro-4H-pyrazolo[4,3- | (m, 2H), 4.07-4.00 (m, 2H), 3.99-3.87 | |
| | c]pyridin-1-yl]-1- | (m, 6H), 3.79-3.66 (m, 3H), 3.62-3.50 | |
| | piperidyl]pyrrolidin-1-yl]-N-[3- | (m, 1H), 3.48-3.36 (m, 1H), 3.26-2.98 | |
| | (3-chloro-4-cyano-phenoxy)- | (m, 3H), 2.93-2.79 (m, 3H), 2.78-2.70 | |
| | 2,2,4,4-tetramethyl- | (m, 1H), 2.39-2.20 (m, 5H), 2.17 | |
| | cyclobutyl]pyrimidine-5- | (s, 1H), 2.12-1.91 (m, 7H), 1.26 (s, 6H), | |
| | carboxamide | 1.22 (s, 6H). | |
| 335 | 2-[(3R)-3-[4-[5-acetyl-3-[7- | (DMSO-d₆) δ = 8.76 (s, 2H), 7.91-7.88 | 961.6 |
| | (difluoromethyl)-6-(1- | (m, 1H), 7.78-7.66 (m, 2H), 7.49 (s, | |
| | methylpyrazol-4-yl)-3,4- | 1H), 7.23-7.18 (m, 1H), 7.10 (s, 1H), | |
| | dihydro-2H-quinolin-1-yl]-6,7- | 7.04-6.97 (m, 1H), 6.96-6.62 (m, 2H), | |
| | dihydro-4H-pyrazolo[4,3-c] | 4.29 (s, 1H), 4.18-4.01 (m, 4H), 3.86 (s, | |
| | pyridin-1-yl]-1-piperidyl] | 3H), 3.81-3.67 (m, 3H), 3.62-3.56 (m, | |
| | pyrrolidin-1-yl]-N-[3-(3-chloro- | 2H), 2.90-2.65 (m, 6H), 2.33 (s, 1H), | |
| | 4-cyano-phenoxy)-2,2,4,4- | 2.24-2.15 (m, 3H), 2.09-1.95 (m, 7H), | |
| | tetramethyl-cyclobutyl] | 1.93-1.77 (m, 4H), 1.27-1.17 (m, 8H), | |
| | pyrimidine-5-carboxamide | 1.11 (s, 6H). | |
| 336 | 4-[(3S)-3-[4-[5-acetyl-3-[7- | δ = 7.74-7.63 (m, 2H), 7.57 -7.50 (m, | 955.7 |
| | (difluoromethyl)-6-(1- | 1H), 7.46 (d, J = 8.4 Hz, 1H), 7.43-7.35 | |
| | methylpyrazol-4-yl)-3,4- | (m, 1H), 7.10-6.82 (m, 2H), 6.71-6.45 | |
| | dihydro-2H-quinolin-1-yl]-6,7- | (m, 4H), 6.44-6.34 (m, 1H), 6.09 (d, J = | |
| | dihydro-4H-pyrazolo[4,3- | 8.0 Hz, 1H), 4.29-4.09 (m, 3H), 4.05 (s, | |
| | c]pyridin-1-yl]-1- | 1H), 4.02-3.86 (m, 9H), 3.82-3.66 (m, | |
| | piperidyl]pyrrolidin-1-yl]-N-[3- | 3H), 3.65-3.49 (m, 2H), 3.44-3.15 (m, | |
| | (4-cyano-3-methoxy-phenoxy)- | 3H), 3.14-2.98 (m, 2H), 2.94-2.79 (m, | |
| | 2,2,4,4-tetramethyl- | 3H), 2.79-2.72 (m, 1H), 2.39-2.22 (m, | |
| | cyclobutyl]benzamide | 4H), 2.210-2.13 (m, 2H), 2.12-2.03 (m, | |
| | | 4H), 2.02-1.93 (m, 2H), 1.27 (s, 6H), | |
| | | 1.24 (s, 6H). | |
| 337 | 4-[(3R)-3-[4-[5-acetyl-3-[7- | (DMSO-d₆) δ = 8.17-8.08 (m, 1H), 7.82-7.71 | 940.7 |
| | (difluoromethyl)-6-(1- | (m, 3H), 7.69-7.57 (m, 1H), 7.50 | |
| | methylpyrazol-4-yl)-3,4- | (s, 1H), 7.46-7.36 (m, 1H), 7.11 (s, 1H), | |
| | dihydro-2H-quinolin-1-yl]-6,7- | 6.98-6.72 (m, 2H), 6.67-6.58 (m, 3H), | |
| | dihydro-4H-pyrazolo[4,3-c] | 6.56-6.46 (m, 1H), 6.30-6.29 (m, 1H), | |
| | pyridin-1-yl]-1-piperidyl] | 4.27 (s, 6H), 3.90 (s, 3H), 3.88-3.84 (m, | |
| | pyrrolidin-1-yl]-N-[3-(4-cyano- | 3H), 3.78-3.68 (m, 3H), 3.64-3.55 (m, | |
| | 3-methoxy-phenoxy)-2,2,4,4- | 4H), 2.92-2.79 (m, 4H), 2.78-2.72 (m, | |
| | tetramethyl-cyclobutyl] | 1H), 2.63-2.53 (m, 2H), 2.42-2.23 (m, | |
| | benzamide | 2H), 2.19-2.04 (m, 5H), 2.02-1.91 (m, | |
| | | 5H), 1.22 (s, 6H), 1.14 (s, 6H). | |

### Example 30: Synthesis of 2-[(3R,4R)-4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3-methyl-1-piperidyl]-N-[3-(3-chloro-4-cyano-phenoxy)-2,2,4,4-tetramethyl-cyclobutyl]pyrimidine-5-carboxamide (Compound 275)

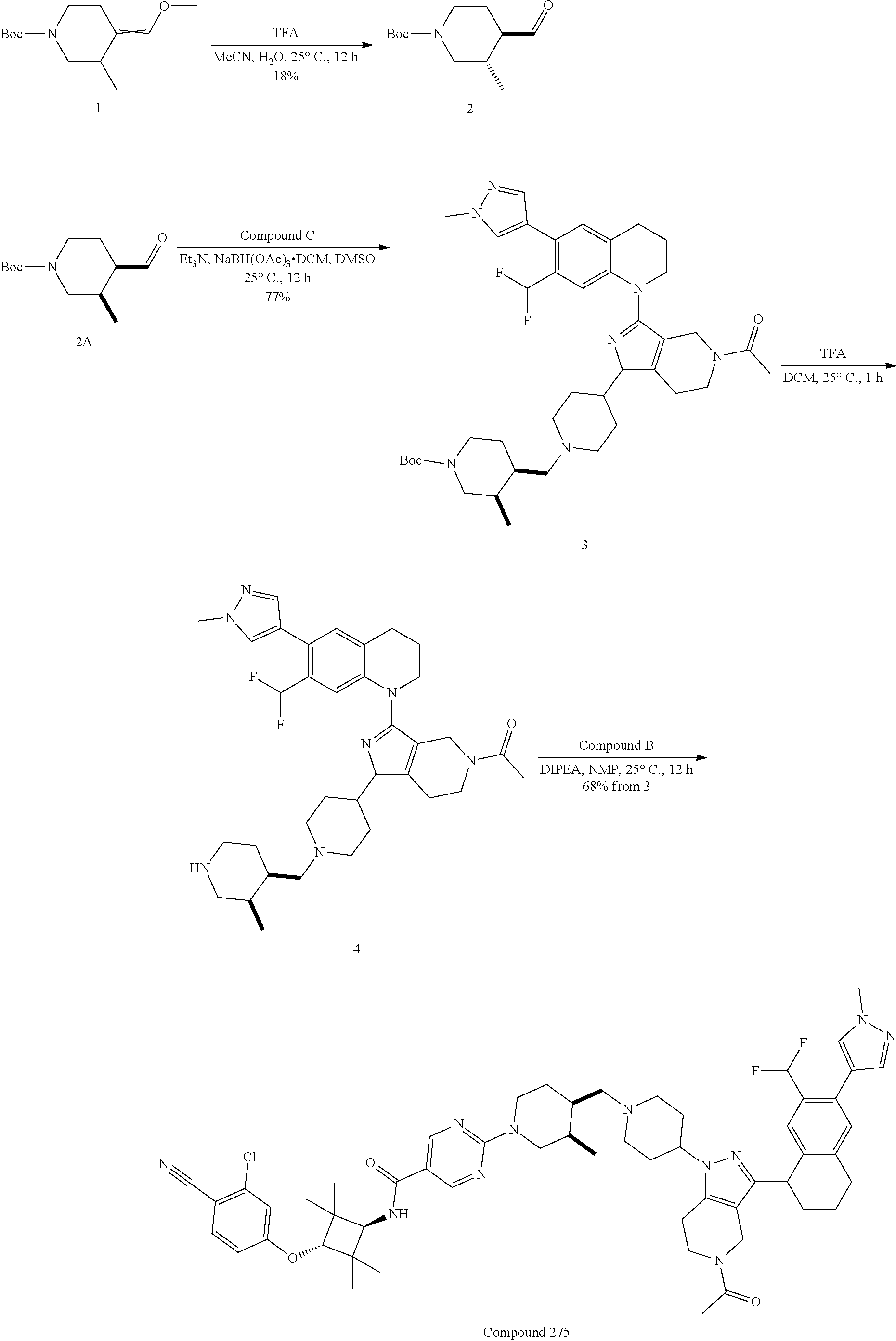

Synthesis of 1 was reported in WO2006/97292 A1

To a solution of tert-butyl (4E)-4-(methoxymethylene)-3-methyl-piperidine-1-carboxylate (10.00 g, 41.4 mmol, 1.0 eq) in MeCN (80 mL) and H₂O (20 mL) was added dropwise TFA (6 mL, 2.0 eq) and the mixture was stirred at 25° C. for 12 h under N₂. The reaction mixture was quenched by addition 10% NaHCO₃ to pH=7.0, and the mixture was dried over Na₂SO₄, filtered and concentrated under reduced pressure to give a residue. The residue was purified by column chromatography (SiO₂, Petroleum ether/Ethyl acetate=20/1 to 10/1). Compound tert-butyl 4-formyl-3-methyl-piperidine-1-carboxylate (1.70 g, 7.48 mmol, 18% yield) was obtained as a white solid.

¹H NMR (400 MHz, DMSO-d₆) δ=9.72 (s, 1H), 4.21-3.85 (m, 1H), 3.83-3.72 (m, 1H), 3.22-3.06 (m, 1H), 3.05-2.79 (m, 1H), 2.59-2.45 (m, 1H), 2.35 (br s, 1H), 1.90-1.75 (m, 1H), 1.74-1.64 (m, 1H), 1.46 (s, 9H), 0.96 (d, J=7.2 Hz, 3H).

To a solution of tert-butyl (3R,4R)-4-formyl-3-methyl-piperidine-1-carboxylate (1.64 g, 7.22 mmol, 1.0 eq), 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (3.68 g, 7.22 mmol, 1.0 eq) in DCM (40 mL) and DMSO (10 mL) was added Et₃N (3.3 mL, 3.3 eq) and NaBH(OAc)₃ (3.28 g, 15.5 mmol, 2.1 eq). The mixture was stirred at 25° C. for 12 h. The reaction mixture was quenched by addition water 50 mL and extracted with dichloromethane (50 mL×3). The orange phase was washed with brine 50 mL, dried over anhydrous sodium sulfate and concentrated under reduced pressure to give residue. The residue was purified by column chromatography (SiO₂, DCM/MeOH=200/1 to 50/1). Compound tert-butyl (3R,4R)-4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3-methyl-piperidine-1-carboxylate (4.00 g, 5.55 mmol, 77% yield) was obtained as a white solid.

¹H NMR (400 MHz, DMSO-d₆) δ=7.75 (s, 1H), 7.49 (s, 1H), 7.10 (s, 1H), 6.96-6.62 (m, 2H), 4.20-4.10 (m, 2H), 4.06-3.90 (m, 2H), 3.86 (s, 3H), 3.78-3.65 (m, 3H), 3.62-3.54 (m, 2H), 3.04-2.67 (m, 8H), 2.22-1.93 (m, 11H), 1.82 (br s, 4H), 1.46-1.14 (m, 11H), 0.75 (br d, J=6.4 Hz, 3H).

LC-MS: MS (ESI⁺) t_{R}=0.542 min, m/z=721.6 [M+H⁺]

SFC: t_{R}=2.350 min, 50%; t_{R}=4.072 min; 50%.

To a solution of tert-butyl (3R,4R)-4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3-methyl-piperidine-1-carboxylate (2.00 g, 2.77 mmol, 1.0 eq) in dichloromethane (10 mL) was added TFA (6.0 mL) and The mixture was stirred at 25° C. for 1 h. The reaction mixture was concentrated under reduced pressure to remove solvent. The residue was diluted with dichloromethane (50 mL), basified with saturated sodium bicarbonate solution (pH=8), extracted with dichloromethane (50 mL×3), The orange phase was washed with brine 50 mL, dried over anhydrous sodium sulfate and concentrated under reduced pressure to give residue. The crude product 1-[3-[7-(difluoromethyl)-6-(1-methylpyrazol-4-yl)-3,4-dihydro-2H-quinolin-1-yl]-1-[i-[(3-methyl-4-piperidyl)methyl]-4-piperidyl]-6,7-dihydro-4H-pyrazolo[4,3-c]pyridin-5-yl]ethanone (1.72 g) as a white solid was used into the next step without further purification.

LC-MS: MS (ESI⁺): t_{R}=0.467 min, m/z=621.5 [M+H⁺]

To a solution of 2-chloro-N-[3-(3-chloro-4-cyano-phenoxy)-2,2,4,4-tetramethyl-cyclobutyl]pyrimidine-5-carboxamide (1.20 g) and 1-[3-[7-(difluoromethyl)-6-(1-methylpyrazol-4-yl)-3,4-dihydro-2H-quinolin-1-yl]-1-[1-[(3-methyl-4-piperidyl)methyl]-4-piperidyl]-6,7-dihydro-4H-pyrazolo[4,3-c]pyridin-5-yl]ethanone (1.72 g, 2.77 mmol, 1.0 eq) in NMP (7.5 mL) was added DIPEA (1.11 g, 8.61 mmol, 1.5 mL, 3.1 eq) and the mixture was stirred at 25° C. for 12 h. The reaction mixture was quenched by addition water 50 mL and extracted with Ethyl acetate (50 mL×3). The orange phase was washed with brine 50 mL, dried over anhydrous sodium sulfate and concentrated under reduced pressure to give residue. The residue was purified by column chromatography (SiO₂, DCM/MeOH=100/1 to 20/1). Compound 2-[(3R,4R)-4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3-methyl-1-piperidyl]-N-[3-(3-chloro-4-cyano-phenoxy)-2,2,4,4-tetramethyl-cyclobutyl]pyrimidine-5-carboxamide (1893.96 mg, 1.89 mmol, 68% yield over two steps) was obtained as an off-white solid.

¹H NMR (400 MHz, DMSO-d₆) δ=8.71 (s, 2H), 7.89 (d, J=8.8 Hz, 1H), 7.75 (s, 1H), 7.69 (d, J=9.2 Hz, 1H), 7.50 (s, 1H), 7.21 (d, J=2.4 Hz, 1H), 7.10 (s, 1H), 7.04-6.97 (m, 1H), 6.95-6.61 (m, 2H), 4.70 (br d, J=13.2 Hz, 1H), 4.58 (br d, J=12.4 Hz, 1H), 4.28 (s, 1H), 4.20-4.10 (m, 2H), 4.02 (br d, J=9.2 Hz, 2H), 3.87 (s, 3H), 3.79-3.66 (m, 2H), 3.64-3.54 (m, 2H), 3.19-3.12 (m, 1H), 3.07-2.96 (m, 2H), 2.95-2.71 (m, 5H), 2.24-1.92 (m, 13H), 1.85 (br s, 2H), 1.55-1.45 (m, 1H), 1.41-1.28 (m, 1H), 1.21 (d, J=3.2 Hz, 6H), 1.10 (s, 6H), 0.74 (br d, J=6.4 Hz, 3H)

LC-MS: MS (ESI⁺): t_{R}=2.087 min, m/z=1003.3 [M+H⁺]

SFC: t_{R}=5.963 min, 46%; t_{R}=6.837 min; 54%.

The compounds below were prepared in a similar manner as described in Example 30.

| | | | LC-MS |
|---|---|---|---|
| Compound | Name | ¹H NMR (DMSO-d₆) | [M + H⁺] |
| 263 | 4-[4-[[4-[5-acetyl-3-[7- | δ = 7.75 (s, 1H), 7.74-7.67 (m, 2H), | 997.8 |
| | (difluoromethyl)-6-(1- | 7.64 (d, J = 8.4 Hz, 1H), 7.50 (s, 1H), | |
| | methylpyrazol-4-yl)-3,4- | 7.46 (d, J = 9.2 Hz, 1H), 7.11 (s, 1H), | |
| | dihydro-2H-quinolin-1-yl]-6,7- | 6.92 (d, J = 8.8 Hz, 2H), 6.82-6.51 (m, | |
| | dihydro-4H-pyrazolo[4,3- | 4H), 4.26 (s, 1H), 4.19-4.10 (m, 2H), | |
| | c]pyridin-1-yl]-1- | 4.04 (d, J = 9.2 Hz, 2H), 3.90 (s, 3H), | |
| | piperidyl]methyl]-3-methyl-1- | 3.87 (s, 3H), 3.76-3.66 (m, 3H), 3.65- | |
| | piperidyl]-N-[3-(4-cyano-3- | 3.57 (m, 3H), 2.95 (d, J = 11.6 Hz, 3H), | |
| | methoxy-phenoxy)-2,2,4,4- | 2.85 (d, J = 6.0 Hz, 4H), 2.76 (d, J = 18.8 | |
| | tetramethyl- | Hz, 1H), 2.20 (s, 2H), 2.09-1.94 (m, | |
| | cyclobutyl]benzamide | 10H), 1.92-1.78 (m, 3H), 1.52 (s, 2H), | |
| | | 1.22 (s, 6H), 1.14 (s, 6H), 0.88 (d, J = 6.8 | |
| | | Hz, 3H). | |
| 363 | 2-[(3R,4R)-4-[4-[5-acetyl-3-[7- | δ = 8.72 (s, 2H), 7.91 (d, J = 8.4 Hz, 1H), | 1003.4 |
| | (difluoromethyl)-6-(1- | 7.76 (s, 1H), 7.70 (br d, J = 9.2 Hz, 1H), | |
| | methylpyrazol-4-yl)-3,4- | 7.51 (s, 1H), 7.22 (d, J = 2.4 Hz, 1H), | |
| | dihydro-2H-quinolin-1-yl]-6,7- | 7.12 (s, 1H), 7.04-6.99 (m, 1H), 6.97- | |
| | dihydro-4H-pyrazolo[4,3-c] | 6.65 (m, 2H), 4.75-4.67 (m, 1H), 4.63- | |
| | pyridin-1-yl]-1-piperidyl] | 4.55 (m, 1H), 4.29 (s, 1H), 4.20-4.11 | |
| | methyl]-3-methyl-1-piperidyl]- | (m, 2H), 4.07-3.99 (m, 2H), 3.88 (s, | |
| | N-[3-(3-chloro-4-cyano- | 3H), 3.78-3.67 (m, 2H), 3.64-3.55 (m, | |
| | phenoxy)-2,2,4,4-tetramethyl- | 2H), 3.21-3.10 (m, 2H), 3.07-2.97 (m, | |
| | cyclobutyl] pyrimidine-5- | 2H), 2.94-2.81 (m, 4H), 2.79-2.72 (m, | |
| | carboxamide | 1H), 2.21-2.15 (m, 2H), 2.09-1.96 (m, | |
| | | 10H), 1.84 (br d, J = 9.6 Hz, 2H), 1.51 | |
| | | (br d, J = 10.4 Hz, 1H), 1.40-1.30 (m, | |
| | | 1H), 1.22 (d, J = 2.8 Hz, 6H), 1.11 (s, | |
| | | 6H), 0.76 (br d, J = 6.4 Hz, 3H). | |
| 364 | 2-[(3S,4S)-4-[4-[5-acetyl-3-[7- | δ = 8.72 (s, 2H), 7.91 (d, J = 8.4 Hz, 1H), | 1003.4 |
| | (difluoromethyl)-6-(1- | 7.76 (s, 1H), 7.70 (br d, J = 9.2 Hz, 1H), | |
| | methylpyrazol-4-yl)-3,4- | 7.51 (s, 1H), 7.22 (d, J = 2.4 Hz, 1H), | |
| | dihydro-2H-quinolin-1-yl]-6,7- | 7.12 (s, 1H), 7.04-6.99 (m, 1H), 6.97- | |
| | dihydro-4H-pyrazolo[4,3-c] | 6.65 (m, 2H), 4.75-4.67 (m, 1H), 4.63- | |
| | pyridin-1-yl]-1-piperidyl] | 4.55 (m, 1H), 4.29 (s, 1H), 4.20-4.11 | |
| | methyl]-3-methyl-1-piperidyl]- | (m, 2H), 4.07-3.99 (m, 2H), 3.88 (s, | |
| | N-[3-(3-chloro-4-cyano- | 3H), 3.78-3.67 (m, 2H), 3.64-3.55 (m, | |
| | phenoxy)-2,2,4,4-tetramethyl- | 2H), 3.21-3.10 (m, 2H), 3.07-2.97 (m, | |
| | cyclobutyl] pyrimidine-5- | 2H), 2.94-2.81 (m, 4H), 2.79-2.72 (m, | |
| | carboxamide | 1H), 2.21-2.15 (m, 2H), 2.09-1.96 (m, | |
| | | 10H), 1.84 (br d, J = 9.6 Hz, 2H), 1.51 | |
| | | (br d, J = 10.4 Hz, 1H), 1.40-1.30 (m, | |
| | | 1H), 1.22 (d, J = 2.8 Hz, 6H), 1.11 (s, | |
| | | 6H), 0.76 (br d, J = 6.4 Hz, 3H). | |

### Example 31: Synthesis of 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,5-dimethyl-1l-piperidyl]-N-[3-(3-chloro-4-cyano-phenoxy)-2,2,4,4-tetramethyl-cyclobutyl]pyrimidine-5-carboxamide (Compound 350)

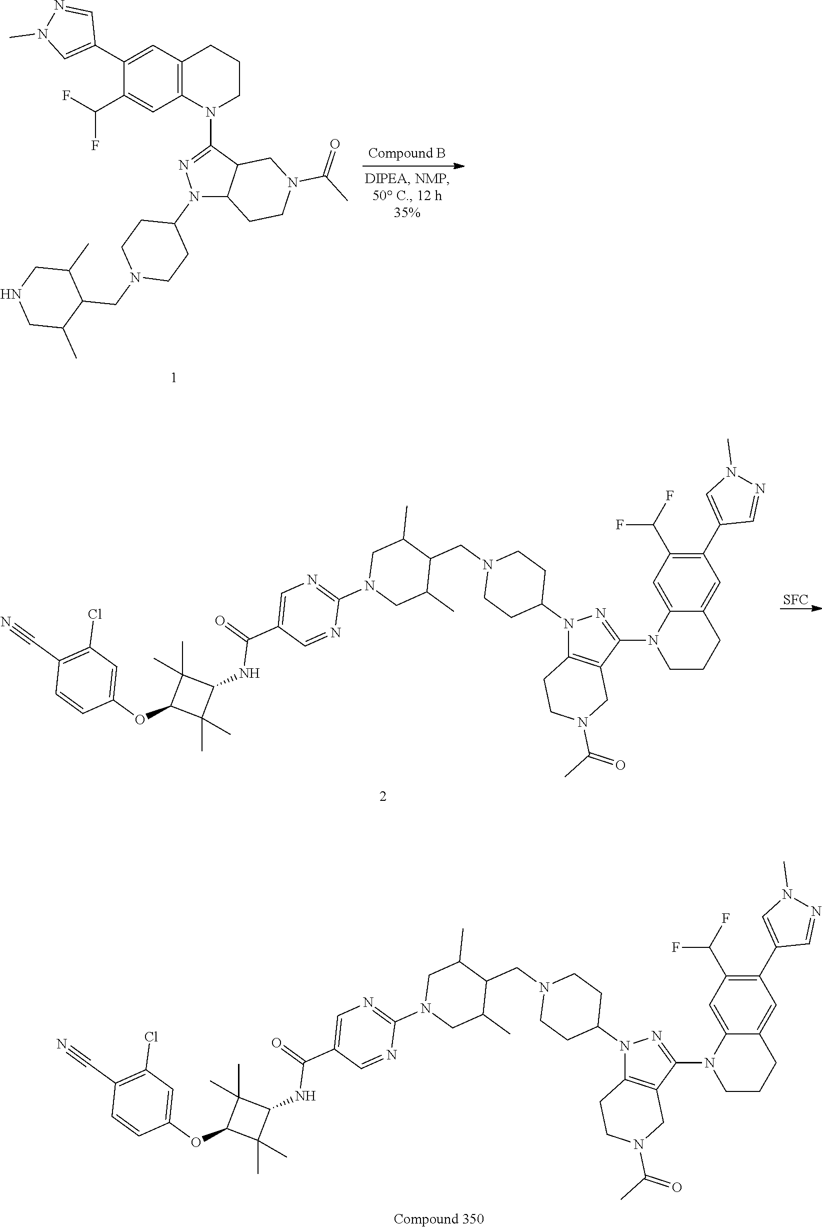

### Synthesis of 1

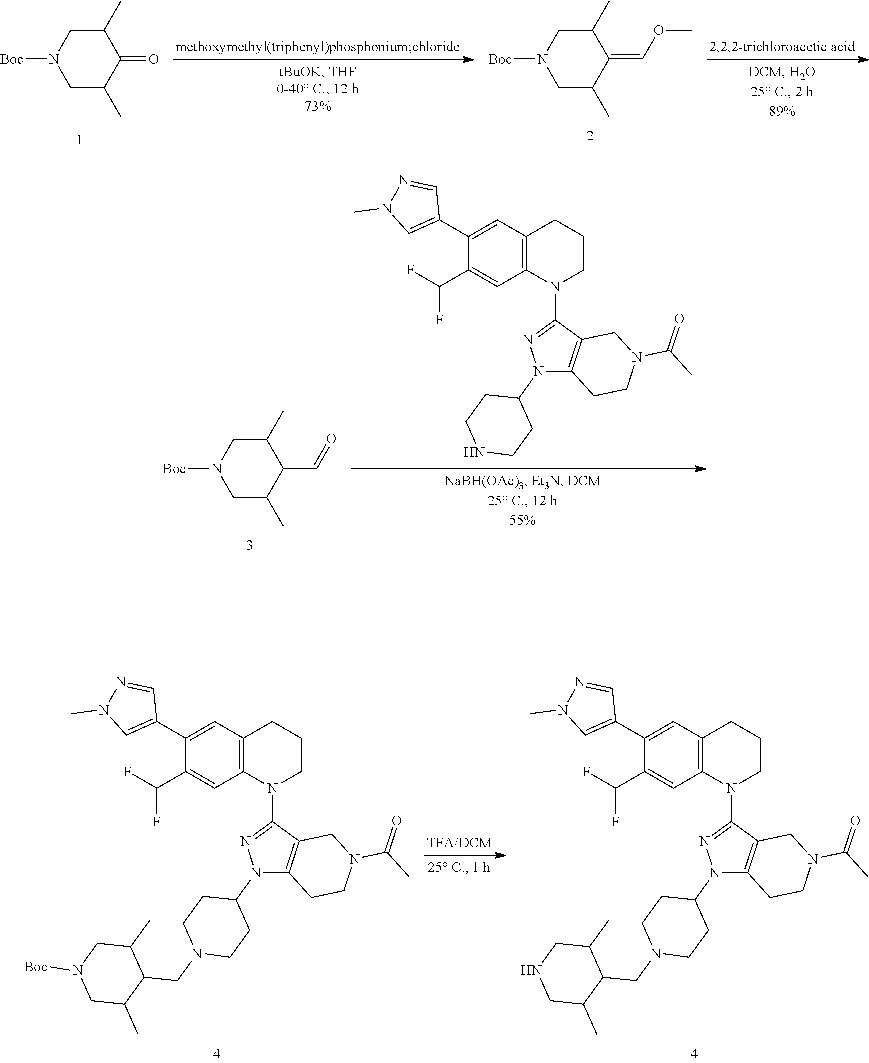 [SMILES: CC1CN(C(=O)OC(C)(C)C)CC(C)C1=O ; COC=C1C(C)CN(C(=O)OC(C)(C)C)CC1C ; CC1CN(C(=O)OC(C)(C)C)CC(C)C1C=O ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(CC3C(C)CN(C(=O)OC(C)(C)C)CC3C)CC2)C1 ; CC(=O)N1CCc2c(c(N3CCCc4cc(-c5cnn(C)c5)c(C(F)F)cc43)nn2C2CCN(CC3C(C)CNCC3C)CC2)C1]

To a solution of methoxymethyl(triphenyl)phosphonium; chloride (15.08 g, 44.0 mmol, 2.0 eq) in THF (150 mL) was added tBuOK (1 M, 66 mL, 3.0 eq) at 0° C. and stirred at 0° C. for 1 h. And then tert-butyl 3,5-dimethyl-4-oxo-piperidine-1-carboxylate (5.00 g, 22.0 mmol, 1.0 eq) in THF (30 mL) was added at 0° C. The reaction mixture was stirred at 40° C. for 11 h under N₂. The mixture was poured into ethyl acetate (300 mL). The aqueous phase was extracted with water (100 mL×3), dried with anhydrous sodium sulfate, concentrated under vacuum. The residue was purified by column chromatography (SiO₂, Petroleum ether/Ethyl acetate=5/1 to 2/1). Compound tert-butyl 4-(methoxymethylene)-3,5-dimethyl-piperidine-1-carboxylate (4.15 g, 16.1 mmol, 73% yield) was obtained as a colorless oil

¹H NMR (400 MHz, CHLOROFORM-d) δ=5.79 (s, 1H), 3.88-3.49 (m, 5H), 3.14-2.72 (m, 3H), 2.28 (d, J=1.2 Hz, 1H), 1.46 (s, 9H), 1.12 (s, 6H)

To a solution of tert-butyl 4-(methoxymethylene)-3,5-dimethyl-piperidine-1-carboxylate (6.80 g, 26.6 mmol, 1.0 eq) in DCM (120 mL) and H₂O (40 mL) was added 2,2,2-trichloroacetic acid (26.11 g, 160 mmol, 6.0 eq). The mixture was stirred at 25° C. for 2 h under N₂. Adjust the PH to 9-10 with NaHCO₃ and then concentrate the system, extracted with DCM (300 mL), wash with water (100 mL×3), dried over Na₂SO₄, concentrated under reduced pressure to give a residue. The residue was purified by column chromatography (SiO₂, Petroleum ether/Ethyl acetate=100/1 to 10/1). Compound tert-butyl 4-formyl-3,5-dimethyl-piperidine-1-carboxylate (5.81 g, 23.8 mmol, 89% yield) was obtained as a colorless oil.

¹H NMR (400 MHz, CHLOROFORM-d) δ=10.11-9.38 (m, 1H), 4.24-3.76 (m, 2H), 2.86 (s 1H), 2.64-2.51 (m, 1H), 2.42-2.19 (m, 1H), 1.99-1.76 (m, 2H), 1.46 (s, 9H), 1.00 (d, J=7.2 Hz, 4H), 0.86 (d, J=6.4 Hz, 2H)

To a solution of tert-butyl 4-formyl-3,5-dimethyl-piperidine-1-carboxylate (268 mg, 1.11 mmol, 1.3 eq) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (435 mg, 854 μmol, 1.0 eq) in DCM (8 mL) was added dropwise Et₃N (864 mg, 8.54 mmol, 10.0 eq) and NaBH(OAc)₃ (905 mg, 4.27 mmol, 5.0 eq) at 25° C. under N₂. The mixture was stirred at 25° C. for 12 h under N₂. The mixture was poured into water (30 mL). The aqueous phase was extracted with DCM (30 mL×3), dried with anhydrous sodium sulfate, concentrated under vacuum. The residue was purified by prep-HPLC (column: Phenomenex luna C18 150×40 mm×15 um; mobile phase: [water(FA)-ACN]; gradient: 32%-62% B over 10 min). Compound tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,5-dimethyl-piperidine-1-carboxylate (364 mg, 471 μmol, 55% yield) was obtained as a yellow solid.

LC-MS: MS (ESI⁺): t_{R}=0.557 min, m/z=735.4 [M+H⁺]

To a solution of tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,5-dimethyl-piperidine-1-carboxylate (364 mg, 471 μmol, 1.0 eq) in DCM (4 mL) was added TFA (1.54 g, 13.5 mmol, 27.3 eq). The mixture was stirred at 25° C. for 1 h under N₂. The reaction mixture was concentrated. Compound 1-[3-[7-(difluoromethyl)-6-(1-methylpyrazol-4-yl)-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (360 mg) as a yellow solid and directly used in the next step without further purification.

LC-MS: MS (ESI⁺): t_{R}=0.471 min, m/z=635.6 [M+H⁺]

### Synthesis of Compound 350:

To a solution of 1-[3-[7-(difluoromethyl)-6-(1-methylpyrazol-4-yl)-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (11 g) and 2-chloro-N-[3-(3-chloro-4-cyano-phenoxy)-2,2,4,4-tetramethyl-cyclobutyl]pyrimidine-5-carboxamide (8.01 g, 19.1 mmol, 1.3 eq) in NMP (100 mL) was added DIPEA (5.70 g, 44.1 mmol, 3.0 eq) at 25° C. The mixture was stirred at 50° C. for 12 h under N₂. The mixture was poured into ethyl acetate (150 mL). The aqueous phase was extracted with water 300 mL (100 mL×3), dried with anhydrous sodium sulfate, concentrated under vacuum. The residue was purified by column chromatography (SiO₂, DCM/MeOH=100/1 to 10/1) as a yellow solid, which was further separated by SFC (column: DAICEL CHIRALPAK AD (250 mm×30 mm, 10 um); mobile phase: [CO₂-ACN/EtOH (0.1% NH₃H₂O)]; B %: 40%, isocratic elution mode). Compound 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (5224.17 mg, 5.08 mmol, 35% yield) was obtained as a yellow solid.

¹H NMR (400 MHz, CHLOROFORM-d) δ=8.68 (s, 2H), 7.59-7.49 (m, 2H), 7.43-7.36 (m, 1H), 7.07-6.93 (m, 2H), 6.89-6.75 (m, 2H), 6.69-6.33 (m, 1H), 5.98 (d, J=8.4 Hz, 1H), 4.29-4.08 (m, 3H), 4.07-3.98 (m, 3H), 3.97-3.85 (m, 5H), 3.77-3.64 (m, 3H), 3.42-3.29 (m, 2H), 3.07 (d, J=10.0 Hz, 2H), 2.93-2.69 (m, 4H), 2.35-2.15 (m, 5H), 2.10-2.00 (m, 6H), 1.86 (s, 5H), 1.24 (s, 6H), 1.21 (s, 6H), 1.01 (d, J=7.2 Hz, 6H)

LC-MS: MS (ESI⁺): t_{R}=3.133 min, m/z=1017.7 [M+H⁺]

The compounds below were prepared in a similar manner as described in Example 31.

| | | | LC-MS |
|---|---|---|---|
| Compound | Name | ¹H NMR (CDCl₃) | [M + H⁺] |
| 327 | 4-[4-[[4-[5-acetyl-3-[7- | δ = 7.75-7.63 (m, 2H), 7.58-7.50 (m, | 1011.7 |
| | (difluoromethyl)-6-(1- | 1H), 7.49-7.44 (m, 1H), 7.43-7.37 (m, | |
| | methylpyrazol-4-yl)-3,4- | 1H), 7.10-6.86 (m, 4H), 6.70-6.36 (m, | |
| | dihydro-2H-quinolin-1-yl]-6,7- | 3H), 6.19-6.06 (m, 1H), 4.34-4.24 (m, | |
| | dihydro-4H-pyrazolo[4,3- | 1H), 4.17-4.12 (m, 2H), 4.08-4.03 (m, | |
| | c]pyridin-1-yl]-1- | 1H), 3.99-3.86 (m, 8H), 3.81-3.65 (m, | |
| | piperidyl]methyl]-3,5-dimethyl- | 3H), 3.43-3.30 (m, 2H), 3.16-3.02 (m, | |
| | 1-piperidyl]-N-[3-(4-cyano-3- | 2H), 2.94-2.72 (m, 6H), 2.32-2.17 (m, | |
| | methoxy-phenoxy)-2,2,4,4- | 5H), 2.14-1.98 (m, 8H), 1.93-1.80 (m, | |
| | tetramethyl- | 3H), 1.32-1.20 (m, 12H), 1.11-1.01 | |
| | cyclobutyl]benzamide | (m, 6H) | |

### Example 32: Synthesis of 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,3-dimethyl-1-piperidyl]-N-[3-(4-cyano-3-methoxy-phenoxy)-2,2,4,4-tetramethyl-cyclobutyl]pyrimidine-5-carboxamide (Compound 360)

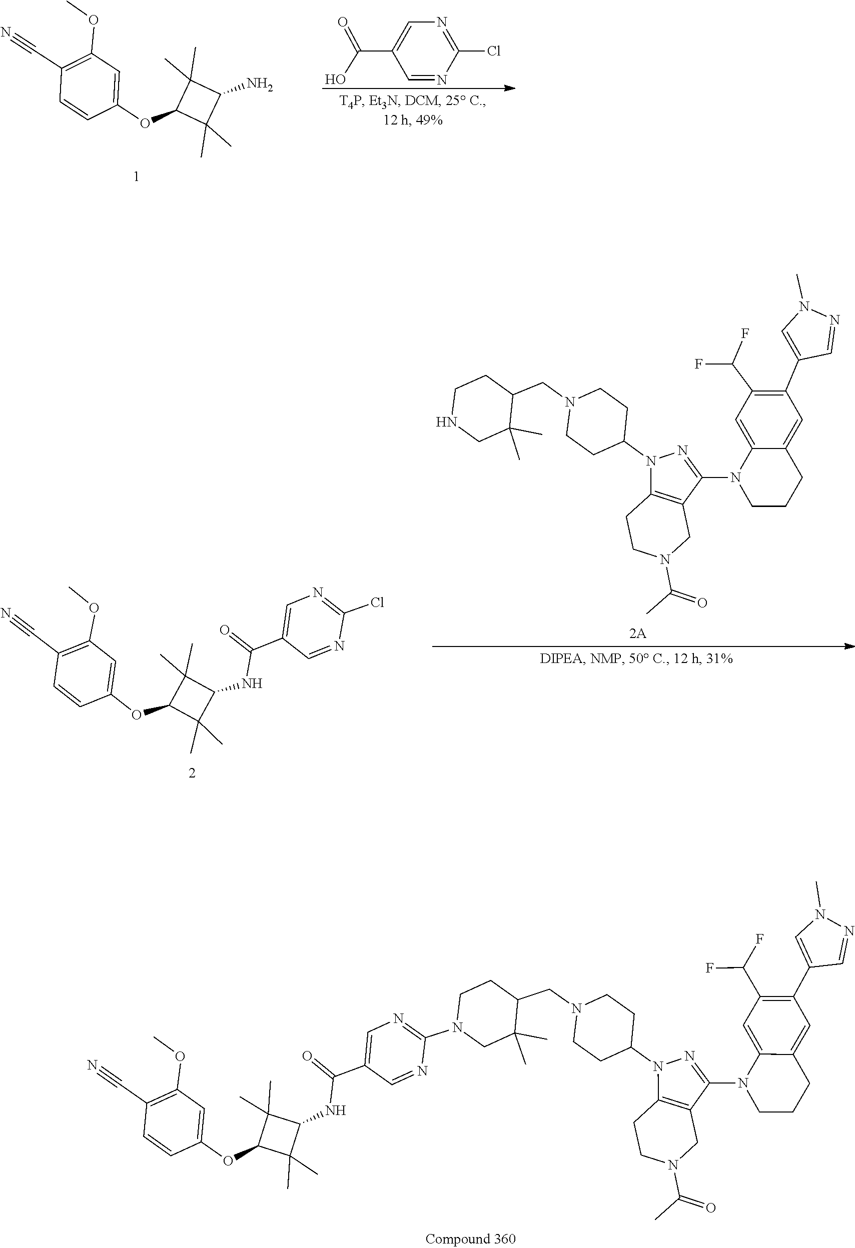 [SMILES: COc1cc(O[C@H]2C(C)(C)[C@H](N)C2(C)C)ccc1C#N ; O=C(O)c1cnc(Cl)nc1 ; COc1cc(O[C@H]2C(C)(C)[C@H](NC(=O)c3cnc(Cl)nc3)C2(C)C)ccc1C#N ; CC(=O)N1CCc2c(c(N3CCCc4cc(-c5cnn(C)c5)c(C(F)F)cc43)nn2C2CCN(CC3CCNCC3(C)C)CC2)C1 ; COc1cc(O[C@H]2C(C)(C)[C@H](NC(=O)c3cnc(N4CCC(CN5CCC(n6nc(N7CCCc8cc(-c9cnn(C)c9)c(C(F)F)cc87)c7c6CCN(C(C)=O)C7)CC5)C(C)(C)C4)nc3)C2(C)C)ccc1C#N]

Synthesis of 1 was described in the report of WO2021/127443 A1

### Synthesis of 2A:

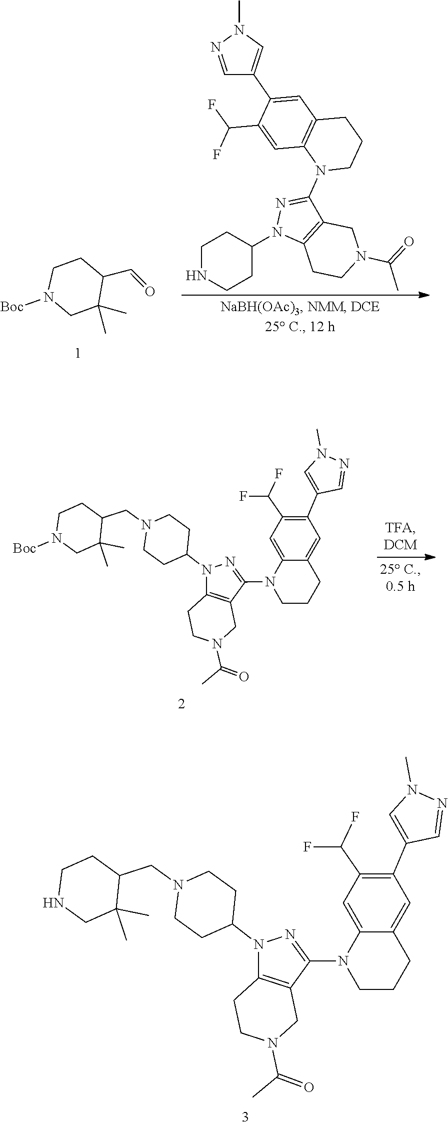 [SMILES: CC(C)(C)OC(=O)N1CCC(C=O)C(C)(C)C1 ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(CC3CCN(C(=O)OC(C)(C)C)CC3(C)C)CC2)C1 ; CC(=O)N1CCc2c(c(N3CCCc4cc(-c5cnn(C)c5)c(C(F)F)cc43)nn2C2CCN(CC3CCNCC3(C)C)CC2)C1]

To a solution of 1-[3-[7-(difluoromethyl)-6-(1-methylpyrazol-4-yl)-3,4-dihydro-2H-quinolin-1-yl]1-(4-piperidyl)-6,7-dihydro-4H-pyrazolo[4,3-c]pyridin-5-yl]ethanone (350 mg, 561.24 μmol, 1 eq, TFA) and NMM (568 mg, 5.61 mmol, 617.04 μL, 10 eq) in DCM (5 mL) was added tert-butyl 4-formyl-3,3-dimenthyl-piperidine-1-carboxylate (270 mg, 1.12 mmol, 1.99 eq) and NaBH(OAc)₃ (238 mg, 1.12 mmol, 2 eq). The reaction mixture was stirred at 25° C. for 12 h. The reaction mixture was concentrated under reduced pressure to give a residue. The residue was purified by column chromatography (silica, Petroleum ether: Ethyl acetate=0:1 to DCM:MeOH=10:1). Compound tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,3-dimethyl-piperidine-1-carboxylate (420 mg, crude) was obtained as off-white solid.

¹H NMR (400 MHz, CDCl₃) δ=7.53 (d, J=5.6 Hz, 1H), 7.40 (d, J=6.0 Hz, 1H), 7.02 (d, J=20.8 Hz, 1H), 6.92-6.81 (m, 1H), 6.52 (dt, J=10.4, 55.6 Hz, 1H), 4.29-4.09 (m, 3H), 3.99-3.87 (m, 5H), 3.79-3.67 (m, 6H), 3.21-3.09 (m, 1H), 3.07-2.95 (m, 1H), 2.92-2.78 (m, 3H), 2.74 (t, J=5.6 Hz, 1H), 2.60 (s, 3H), 2.52-2.35 (m, 5H), 2.32-2.13 (m, 5H), 2.11-2.03 (m, 9H), 2.01-1.90 (m, 2H), 1.84-1.75 (m, 1H), 0.97-0.90 (m, 3H), 0.79 (s, 3H)

LC-MS: MS (ESI⁺): t_{R}=0.488 min, m/z=735.4 [M+H⁺]

To a solution of tert-butyl 4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,3-dimethyl-piperidine-1-carboxylate (120 mg, 163.28 μmol, 1 eq) in DCM (5 mL) was added TFA (93 mg, 816.42 μmol, 60.65 μL, 5 eq). The reaction mixture was stirred at 25° C. for 0.5 h. The reaction mixture was concentrated under reduced pressure to give a residue. Compound 1-[3-[7-(difluoromethyl)-6-(1-methylpyrazol-4-yl)-3,4-dihydro-2H-quinolin-1-yl]-1-[1-[(3,3-dimethyl-4-piperidyl)methyl]-4-piperidyl]-6,7-dihydro-4H-pyrazolo[4,3-c]pyridin-5-yl]ethanone (104 mg, crude) was obtained as yellow oil and directly used in the next step without further purification.

LC-MS: MS (ESI⁺): t_{R}=0.401 min, m/z=635.4 [M+H⁺]

### Synthesis of Compound 360:

To a solution of 4-(3-amino-2,2,4,4-tetramethyl-cyclobutoxy)-2-methoxy-benzonitrile (2.00 g, 7.29 mmol, 1.0 eq) in dichloromethane (20 mL) was added Et₃N (5.90 g, 58.32 mmol, 8.12 mL, 8.0 eq) and 2-chloropyrimidine-5-carboxylic acid (1.16 g, 7.29 mmol, 1.0 eq), T₄P (3.94 g, 10.93 mmol, 1.5 eq). The mixture was stirred at 25° C. for 12 h. The mixture was poured into water (50 mL). The aqueous phase was extracted with dichloromethane (50 mL×2). The combined organic phase was washed with brine (100 mL×3), dried with anhydrous sodium sulfate, filtered and concentrated in vacuum. The residue was purified by silica gel chromatography (0%-40% ethyl acetate in petroleum ether). Compound 2-chloro-N-[3-(4-cyano-3-methoxy-phenoxy)-2,2,4,4-tetramethyl-cyclobutyl]pyrimidine-5-carboxamide (1.49 g, 3.59 mmol, 49% yield) was obtained as a white solid

¹H NMR (400 MHz, DMSO) δ=9.09 (s, 2H), 8.30 (d, J=9.0 Hz, 1H), 7.64 (d, J=8.5 Hz, 1H), 6.70-6.44 (m, 2H), 4.06 (d, J=9.1 Hz, 1H), 3.91 (s, 3H), 1.27-1.11 (m, 12H)

LC-MS: MS (ESI⁺): t_{R}=0.543 min, m/z=415.1 [M+H⁺]

To a solution of 1-[3-[7-(difluoromethyl)-6-(1-methylpyrazol-4-yl)-3,4-dihydro-2H-quinolin-1-yl]-1-[1-[(3,3-dimethyl-4-piperidyl)methyl]-4-piperidyl]-6,7-dihydro-4H-pyrazolo[4,3-c]pyridin-5-yl]ethanone (129 mg, 203.21 μmol, 1.0 eq) in NMP (3 mL) was added DIPEA (210 mg, 1.63 mmol, 283.16 μL, 8.0 eq) and 2-chloro-N-[3-(4-cyano-3-methoxy-phenoxy)-2,2,4,4-tetramethyl-cyclobutyl]pyrimidine-5-carboxamide (126 mg, 304.82 μmol, 1.5 eq). The mixture was stirred at 50° C. for 12 hours. The mixture was poured into water (50 mL). The aqueous phase was extracted with ethyl acetate (50 mL×2). The combined organic phase was washed with brine (100 mL×3), dried with anhydrous sodium sulfate, filtered and concentrated in vacuum. The residue was purified by prep-TLC (SiO₂, Dichloromethane:Methanol=15:1). The residue was purified by prep-HPLC (column: Unisil 3-100 C18 Ultra 150*50 mm*3 um; mobile phase: [water (FA)-ACN]; gradient: 30%-60% B over 10 min) to give desired compound. Then it was lyophilized. Compound 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,3-dimethyl-1-piperidyl]-N-[3-(4-cyano-3-methoxy-phenoxy)-2,2,4,4-tetramethyl-cyclobutyl]pyrimidine-5-carboxamide (65.78 mg, 64.26 μmol, 31% yield) was obtained as a white solid.

¹H NMR (400 MHz, CDCl₃) δ=8.68 (s, 2H), 7.54 (d, J=5.8 Hz, 1H), 7.46 (d, J=8.6 Hz, 1H), 7.41 (d, J=6.6 Hz, 1H), 7.09-6.98 (m, 1H), 6.93-6.84 (m, 1H), 6.69-6.38 (m, 3H), 5.92 (d, J=8.3 Hz, 1H), 4.87 (d, J=13.5 Hz, 1H), 4.50 (d, J=13.0 Hz, 1H), 4.29-4.10 (m, 3H), 4.04 (s, 1H), 3.96 (d, J=1.9 Hz, 4H), 3.93-3.89 (m, 4H), 3.80-3.66 (m, 3H), 3.25-3.08 (m, 1H), 3.05-2.94 (m, 1H), 2.93-2.84 (m, 3H), 2.82 (t, J=5.5 Hz, 1H), 2.77-2.69 (m, 2H), 2.51-2.40 (m, 1H), 2.37-2.21 (m, 3H), 2.17 (s, 2H), 2.12-2.02 (m, 5H), 1.98 (d, J=10.5 Hz, 3H), 1.61-1.56 (m, 1H), 1.44-1.35 (m, 1H), 1.24 (d, J=7.1 Hz, 12H), 1.05 (s, 3H), 0.79 (s, 3H)

LC-MS: MS (ESI⁺): t_{R}=3.050 min, m/z=1013.8 [M+H⁺]

The compounds below were prepared in a similar manner as described in Example 32.

| | | | LC-MS |
|---|---|---|---|
| Compound | Name | ¹H NMR (DMSO-d₆) | [M + H⁺] |
| 329 | 2-[4-[[4-[5-acetyl-3-[7- | δ = 8.72 (s, 2H), 7.90 (d, J = 8.8 Hz, 1H), | 1019.5 |
| | (difluoromethyl)-6-(1- | 7.75 (s, 1H), 7.70 (d, J = 9.4 Hz, 1H), | |
| | methylpyrazol-4-yl)-3,4- | 7.50 (s, 1H), 7.21 (d, J = 2.4 Hz, 1H), | |
| | dihydro-2H-quinolin-1-yl]-6,7- | 7.10 (s, 1H), 7.04-6.98 (m, 1H), 6.96- | |
| | dihydro-4H-pyrazolo[4,3-c] | 6.63 (m, 2H), 5.09 (br d, J = 13.6 Hz, | |
| | pyridin-1-yl]-1-piperidyl] | 1H), 4.83-4.71 (m, 1H), 4.29 (s, 1H), | |
| | methyl]-3-methoxy-1- | 4.20-4.10 (m, 2H), 4.07-3.98 (m, 2H), | |
| | piperidyl]-N-[3-(3-chloro-4- | 3.86 (s, 3H), 3.78-3.66 (m, 2H), 3.62- | |
| | cyano-phenoxy)-2,2,4,4- | 3.55 (m, 2H), 3.24 (s, 3H), 3.02-2.81 | |
| | tetramethyl-cyclobutyl] | (m, 8H), 2.77-2.71 (m, 1H), 2.25-1.92 | |
| | pyrimidine-5-carboxamide | (m, 12H), 1.90-1.80 (m, 2H), 1.56- | |
| | | 1.47 (m, 1H), 1.42-1.32 (m, 1H), 1.21 | |
| | | (s, 6H), 1.11 (s, 6H). | |
| 331 | 2-[4-[[4-[5-acetyl-3-[7- | (CDCl₃) δ = 8.71 (s, 2 H) 7.58 (d, J = 8.8 | 1019.7 |
| | (difluoromethyl)-6-(1- | Hz, 1 H) 7.54 (d, J = 5.2 Hz, 1 H) 7.41 (d, | |
| | methylpyrazol-4-yl)-3,4- | J = 6.4 Hz, 1 H) 7.03 (d, J = 20.0 Hz, 1 H) | |
| | dihydro-2H-quinolin-1-yl]-6,7- | 6.97 (d, J = 2.4 Hz, 1 H) 6.93-6.85 (m, 1 | |
| | dihydro-4H-pyrazolo[4,3- | H) 6.81 (dd, J = 8.8, 2.4 Hz, 1 H) 6.69- | |
| | c]pyridin-1-yl]-1- | 6.38 (m, 1 H) 5.95 (d, J = 8.4 Hz, 1 H) | |
| | piperidyl]methyl]-3-methoxy-1- | 4.96-4.73 (m, 1 H) 4.63-4.46 (m, 1 H) | |
| | piperidyl]-N-[3-(3-chloro-4- | 4.28 (s, 1 H) 4.16-4.12 (m, 2 H) 4.05 (s, | |
| | cyano-phenoxy)-2,2,4,4- | 1 H) 3.96 (d, J = 1.6 Hz, 4 H) 3.92-3.88 | |
| | tetramethyl- | (m, 1 H) 3.77-3.66 (m, 3 H) 3.46 (s, 3 H) | |
| | cyclobutyl]pyrimidine-5- | 3.21-3.09 (m, 2 H) 3.07-3.00 (m, 2 H) | |
| | carboxamide | 2.90-2.84 (m, 2 H) 2.81 (t, J = 5.6 Hz, 1 | |
| | | H) 2.76-2.73 (m, 1 H) 2.39-2.19 (m, 4 H) | |
| | | 2.17 (s, 2 H) 2.09 (d, J = 6.0 Hz, 2 H) | |
| | | 2.07-2.02 (m, 3 H) 1.96-1.85 (m, 2 H) | |
| | | 1.80-1.67 (m, 3 H) 1.28 (s, 1 H) 1.26 (s, | |
| | | 6 H) 1.22 (s, 6 H). | |
| 332 | 4-[4-[[4-[5-acetyl-3-[7- | δ = 7.70 (d, J = 8.4 Hz, 2H), 7.54 (d, J = | 1013.8 |
| | (difluoromethyl)-6-(1- | 5.6 Hz, 1H), 7.46 (d, J = 8.4 Hz, 1H), | |
| | methylpyrazol-4-yl)-3,4- | 7.41 (d, J = 6.4 Hz, 1H), 7.08-6.98 (m, | |
| | dihydro-2H-quinolin-1-yl]-6,7- | 1H), 6.92 (d, J = 8.4 Hz, 2H), 6.87 (d, | |
| | dihydro-4H-pyrazolo[4,3- | J = 4.0 Hz, 1H), 6.69-6.36 (m, 3H), 6.12 | |
| | c]pyridin-1-yl]-1- | (d, J = 8.0 Hz, 1H), 4.28-4.03 (m, 4H), | |
| | piperidyl]methyl]-3-methoxy-1- | 4.03-3.87 (m, 9H), 3.82-3.61 (m, 4H), | |
| | piperidyl]-N-[3-(4-cyano-3- | 3.46 (s, 3H), 3.17-3.03 (m, 2H), 3.02- | |
| | methoxy-phenoxy)-2,2,4,4- | 2.93 (m, 1H), 2.93-2.78 (m, 4H), 2.78- | |
| | tetramethyl- | 2.67 (m, 3H), 2.36-2.19 (m, 4H), 2.19- | |
| | cyclobutyl]benzamide | 2.01 (m, 7H), 1.92 (s, 2H), 1.44-1.35 | |
| | | (m, 1H), 1.25 (d, J = 10.0 Hz, 12H). | |
| 356 | 2-[4-[[4-[5-acetyl-3-[7- | (CDCl₃) δ = 8.68 (s, 2H), 7.59-7.53 (m, | 1017.7 |
| | (difluoromethyl)-6-(1- | 2H), 7.44-7.38 (m, 1H), 7.06-6.99 (m, | |
| | methylpyrazol-4-yl)-3,4- | 1H), 6.97 (d, J = 2.4 Hz, 1H), 6.89-6.86 | |
| | dihydro-2H-quinolin-1-yl]-6,7- | (m, 1H), 6.81 (dd, J = 2.4, 8.7 Hz, 1H), | |
| | dihydro-4H-pyrazolo[4,3- | 6.69-6.37 (m, 1H), 5.91 (d, J = 8.1 Hz, | |
| | c]pyridin-1-yl]-1- | 1H), 4.86 (d, J = 12.9 Hz, 1H), 4.49 (d, | |
| | piperidyl]methyl]-3,3-dimethyl- | J = 13.0 Hz, 1H), 4.26 (s, 1H), 4.14- | |
| | 1-piperidyl]-N-[3-(3-chloro-4- | 4.11 (m, 2H), 4.04 (s, 1H), 3.96 (d, J = | |
| | cyano-phenoxy)-2,2,4,4- | 2.0 Hz, 3H), 3.91 (t, J = 5.7 Hz, 2H), | |
| | tetramethyl- | 3.78-3.68 (m, 3H), 3.21-3.05 (m, 1H), | |
| | cyclobutyl]pyrimidine-5- | 2.98-2.80 (m, 5H), 2.77-2.70 (m, 2H), | |
| | carboxamide | 2.47-2.37 (m, 1H), 2.34-2.21 (m, 3H), | |
| | | 2.17 (s, 1H), 2.13-2.04 (m, 5H), 2.02- | |
| | | 1.87 (m, 4H), 1.58-1.53 (m, 1H), 1.42- | |
| | | 1.35 (m, 1H), 1.25 (s, 6H), 1.22 (s, 6H), | |
| | | 1.05 (s, 3H), 0.79 (s, 3H) | |

### Example 33: Synthesis of methyl (7S)-2-benzyl-3-[(1R,3R)-3-[4-[5-[[3-(3-chloro-4-cyano-phenoxy)-2,2,4,4-tetramethyl-cyclobutyl]carbamoyl]pyrimidin-2-yl]piperazine-1-carbonyl]cyclohexyl]-7-methyl-8,9-dihydro-7H-imidazo[4,5-f]quinoline-6-carboxylate (Compound 127) and methyl (7S)-2-benzyl-3-[(1S,3S)-3-[4-[5-[[3-(3-chloro-4-cyano-phenoxy)-2,2,4,4-tetramethyl-cyclobutyl]carbamoyl]pyrimidin-2-yl]piperazine-1-carbonyl]cyclohexyl]-7-methyl-8,9-dihydro-7H-imidazo[4,5-f]quinoline-6-carboxylate (Compound 315)

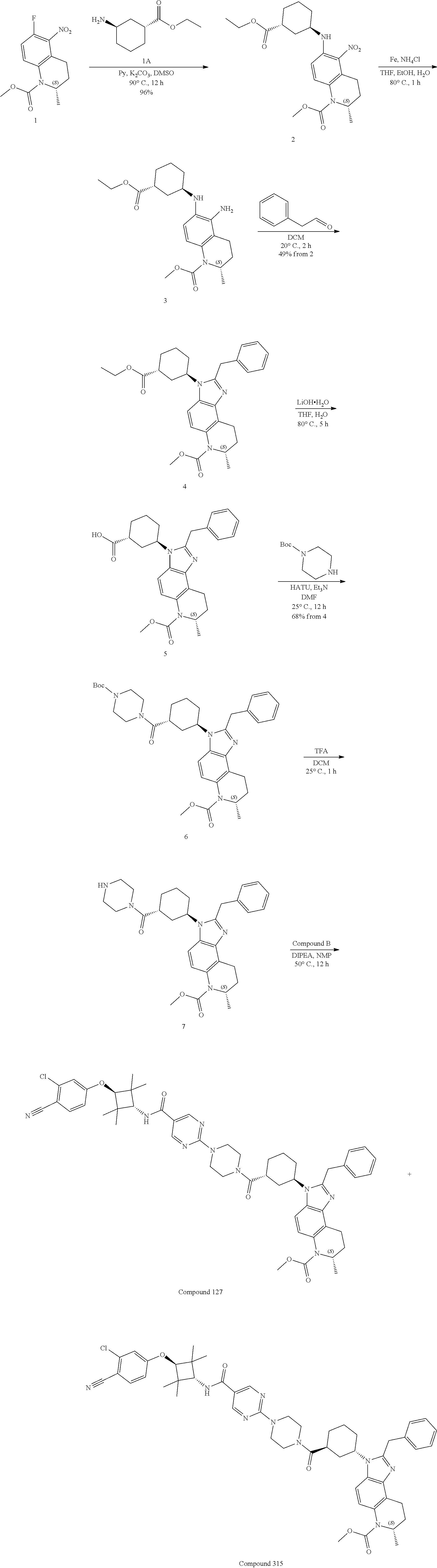

Synthesis of 1A was reported in WO2023/110936 A1

To a solution of methyl (2S)-6-fluoro-2-methyl-5-nitro-3,4-dihydro-2H-quinoline-1-carboxylate (330 mg, 1.23 mmol, 1.0 eq) and ethyl (1R,3R)-3-aminocyclohexanecarboxylate (316 mg, 1.85 mmol, 1.5 eq) in DMSO (12 mL) was added pyridine (292 mg, 3.69 mmol, 298 μL, 3.0 eq) and K₂CO₃ (340 mg, 2.46 mmol, 2.0 eq). The reaction mixture was stirred at 90° C. for 12 h. The mixture was poured into iced water (120 mL). The aqueous phase was extracted with ethyl acetate (40 mL×3). The combined organic phase was washed with brine (40 mL×2), dried with anhydrous sodium sulfate, filtered and concentrated under vacuum. The residue was purified by silica gel column chromatography (petroleum ether/ethyl acetate=10/1 to 3/1). Compound methyl (2S)-6-[[(1R,3R)-3-ethoxycarbonylcyclohexyl]amino]-2-methyl-5-nitro-3,4-dihydro-2H-quinoline-1-carboxylate (500 mg, 1.18 mmol, 96% yield) was obtained as a red gum.

LC-MS: MS (ESI⁺: t_{R}=1.066 min, m/z=420.2 [M−H⁺]

To a mixture of methyl (2S)-6-[[(1R,3R)-3-ethoxycarbonylcyclohexyl]amino]-2-methyl-5-nitro-3,4-dihydro-2H-quinoline-1-carboxylate (400 mg, 954 μmol, 1.0 eq) and NH₄Cl (480 mg, 8.97 mmol, 9.4 eq) in THF (6 mL) and EtOH (6 mL) and H₂O (4 mL) was added Fe (480 mg, 8.59 mmol, 490 μL, 9.0 eq). The reaction mixture was stirred at 80° C. for 1 h under N₂. The reaction mixture was filtered and washed with EtOAc (20 mL×3). The collected filtrate was concentrated to give a residue. Compound methyl (2S)-5-amino-6-[[(1R,3R)-3-ethoxycarbonylcyclohexyl]amino]-2-methyl-3,4-dihydro-2H-quinoline-1-carboxylate (340 mg) was obtained as a colorless oil and directly used in the next step without further purification.

LC-MS: MS (ESI: t_{R}=0.511 min, m/z=390.3 [M+H⁺]

To a mixture of methyl (2S)-5-amino-6-[[(1R,3R)-3-ethoxycarbonylcyclohexyl]amino]-2-methyl-3,4-dihydro-2H-quinoline-1-carboxylate (340 mg) in DCM (20 mL) was added 2-phenylacetaldehyde (340 mg, 2.83 mmol, 221 μL, 3.2 eq). The reaction mixture was stirred at 20° C. for 2 h under N₂. The mixture was concentrated to give a residue. The residue was purified by silica gel column chromatography (petroleum ether/ethyl acetate=5/1 to 2/1). Compound methyl (7S)-2-benzyl-3-[(1R,3R)-3-ethoxycarbonylcyclohexyl]-7-methyl-8,9-dihydro-7H-imidazo[4,5-f]quinoline-6-carboxylate (210 mg, 428.9 μmol, 49% yield over two steps) was obtained as a colorless gum.

LC-MS: MS (ESI: t_{R}=0.578 min, m/z=490.3 [M+H⁺]

To a mixture of methyl (7S)-2-benzyl-3-[(1R,3R)-3-ethoxycarbonylcyclohexyl]-7-methyl-8,9-dihydro-7H-imidazo[4,5-f]quinoline-6-carboxylate (150 mg, 306 μmol, 1.0 eq) in THF (2 mL) was added LiOH H₂O (129 mg, 3.1 mmol, 10.0 eq) in H₂O (2 mL). The reaction mixture was stirred at 80° C. for 5 h. The reaction mixture was concentrated to removed THF, poured into iced water (15 mL), adjusted to pH=6 with hydrochloric acid (1 M) at 0° C., and lyophilization to give a residue. Compound (1R,3R)-3-[(7S)-2-benzyl-6-methoxycarbonyl-7-methyl-8,9-dihydro-7H-imidazo[4,5-f]quinolin-3-yl]cyclohexanecarboxylic acid (140 mg) was obtained as a off-white solid and directly used in the next step without further purification.

To a mixture of (1R,3R)-3-[(7S)-2-benzyl-6-methoxycarbonyl-7-methyl-8,9-dihydro-7H-imidazo[4,5-f]quinolin-3-yl]cyclohexanecarboxylic acid (140 mg) in DMF (6 mL) was added Et₃N (92 mg, 910 μmol, 127 μL, 3.0 eq), tert-butyl piperazine-1-carboxylate (85 mg, 455 μmol, 1.5 eq) and HATU (231 mg, 607 μmol, 2.0 eq). The reaction mixture was stirred at 25° C. for 12 h. The mixture was poured into iced water (80 mL). The aqueous phase was extracted with ethyl acetate (25 mL×3). The combined organic phase was washed with brine (20 mL×2), dried with anhydrous sodium sulfate, filtered and concentrated under vacuum. The residue was purified by prep-TLC (petroleum ether/ethyl acetate=1/1). Compound methyl (7S)-2-benzyl-3-[(1R,3R)-3-(4-tert-butoxycarbonylpiperazine-1-carbonyl)cyclohexyl]-7-methyl-8,9-dihydro-7H-imidazo[4,5-f]quinoline-6-carboxylate (140 mg, 207 μmol, 68% yield over two steps) was obtained as a brown gum.

LC-MS: MS (ESI⁺): t_{R}=0.574 min, m/z=630.6 [M+H⁺]

To a mixture of methyl (7S)-2-benzyl-3-[(1R,3R)-3-(4-tert-butoxycarbonylpiperazine-1-carbonyl)cyclohexyl]-7-methyl-8,9-dihydro-7H-imidazo[4,5-f]quinoline-6-carboxylate (140 mg, 222 μmol, 1.0 eq) in DCM (4 mL) was added TFA (3.07 g, 26.9 mmol, 2 mL, 121.0 eq). The reaction mixture was stirred at 25° C. for 1 h. The mixture was concentrated to give a residue. Compound methyl (7S)-2-benzyl-7-methyl-3-[(1R,3R)-3-(piperazine-1-carbonyl)cyclohexyl]-8,9-dihydro-7H-imidazo[4,5-f]quinoline-6-carboxylate (140 mg) was obtained as a brown gum and directly used in the next step without further purification.

LC-MS: MS (ESI⁺): t_{R}=0.763 min, 0.777 min, m/z=530.4 [M+H⁺]

To a mixture of methyl (7S)-2-benzyl-7-methyl-3-[(1R,3R)-3-(piperazine-1-carbonyl)cyclohexyl]-8,9-dihydro-7H-imidazo[4,5-f]quinoline-6-carboxylate (140 mg) in NMP (2 mL) was added DIPEA (141 mg, 1.09 mmol, 189 μL, 5.0 eq) and 2-chloro-N-[3-(3-chloro-4-cyano-phenoxy)-2,2,4,4-tetramethyl-cyclobutyl]pyrimidine-5-carboxamide (110 mg, 262 mol, 1.2 eq). The reaction mixture was stirred at 50° C. for 12 h. The mixture was diluted with EtOAc (100 mL), washed with water (15 mL×2), brine (15 mL), dried with anhydrous sodium sulfate, filtered and concentrated under vacuum. The residue was purified by semi-preparative reverse phase HPLC (column: Phenomenex luna C18 150×25 mm×10 um; mobile phase: [water (TFA)-ACN]; gradient: 45%-75% B over 10 min) and prep-TLC (ethyl acetate/methyl alcohol=40/1).

Compound methyl (7S)-2-benzyl-3-[(1R,3R)-3-[4-[5-[[3-(3-chloro-4-cyano-phenoxy)-2,2,4,4-tetramethyl-cyclobutyl]carbamoyl]pyrimidin-2-yl]piperazine-1-carbonyl]cyclohexyl]-7-methyl-8,9-dihydro-7H-imidazo[4,5-f]quinoline-6-carboxylate (27.09 mg, 29.55 μmol, 14% yield over two steps) was obtained as a white solid.

¹H NMR (400 MHz, CHLOROFORM-d) δ=8.75 (s, 2H) 7.58 (d, J=8.8 Hz, 1H) 7.32-7.48 (m, 3H) 7.30 (s, 1H) 7.24 (s, 2H) 7.13-7.20 (m, 1H) 6.97 (s, 1H) 6.81 (m, 1H) 5.95 (m, 1H) 5.20-5.39 (m, 1H) 4.75-4.85 (m, 1H) 4.43-4.56 (m, 1H) 4.35-4.42 (m, 1H) 4.14 (d, J=7.6 Hz, 1H) 4.06 (s, 1H) 3.95 (s, 4H) 3.79 (s, 3H) 3.69-3.78 (m, 2H) 3.46-3.60 (m, 2H) 3.24-3.37 (m, 1H) 3.18 (s, 1H) 2.99-3.12 (m, 1H) 2.20-2.39 (m, 2H) 1.93-2.07 (m, 2H) 1.86 (m, 1H) 1.74-1.81 (m, 1H) 1.64-1.72 (m, 2H) 1.32-1.40 (m, 1H) 1.25 (d, J=14.4 Hz, 12H) 1.20 (d, J=6.4 Hz, 3H).

LC-MS: MS (ESI⁺): t_{R}=2.301 min, m/z=912.4 [M+H⁺]

Compound methyl (7S)-2-benzyl-3-[(1S,3S)-3-[4-[5-[[3-(3-chloro-4-cyano-phenoxy)-2,2,4,4-tetramethyl-cyclobutyl]carbamoyl]pyrimidin-2-yl]piperazine-1-carbonyl]cyclohexyl]-7-methyl-8,9-dihydro-7H-imidazo[4,5-f]quinoline-6-carboxylate (33.33 mg, 36.27 μmol, 17% yield over two steps) was obtained as a white solid.

¹H NMR (400 MHz, CHLOROFORM-d) δ=8.75 (s, 2H) 7.58 (d, J=8.8 Hz, 1H) 7.31-7.44 (m, 3H) 7.30 (s, 1H) 7.22-7.26 (m, 2H) 7.14-7.20 (m, 1H) 6.97 (d, J=2.0 Hz, 1H) 6.78-6.84 (m, 1H) 5.91-5.98 (m, 1H) 5.21-5.40 (m, 1H) 4.75-4.87 (m, 1H) 4.43-4.65 (m, 1H) 4.31-4.42 (m, 1H) 4.15 (d, J=8.0 Hz, 1H) 4.06 (s, 1H) 3.89-4.03 (m, 4H) 3.69-3.85 (m, 5H) 3.48-3.64 (m, 2H) 3.23-3.38 (m, 1H) 3.16-3.22 (m, 1H) 3.02-3.15 (m, 1H) 2.21-2.35 (m, 2H) 1.94-2.14 (m, 2H) 1.82-1.91 (m, 1H) 1.64-1.81 (m, 3H) 1.38-1.49 (m, 1H) 1.25 (d, J=14.4 Hz, 12H) 1.18 (d, J=6.4 Hz, 3H).

LC-MS: MS (ESI⁺): t_{R}=2.276 min, m/z=912.6 [M+H⁺]

### Example 34: Synthesis of 4-[4-[2-[4-[8-(5-acetyl-1-tetrahydropyran-4-yl-6,7-dihydro-4H-pyrazolo[4,3-c]pyridin-3-yl)-3-isoquinolyl]pyrazol-1-yl]ethyl]-1-piperidyl]-N-[3-(4-cyano-3-methoxy-phenoxy)-2,2,4,4-tetramethyl-cyclobutyl]benzamide (Compound 244)

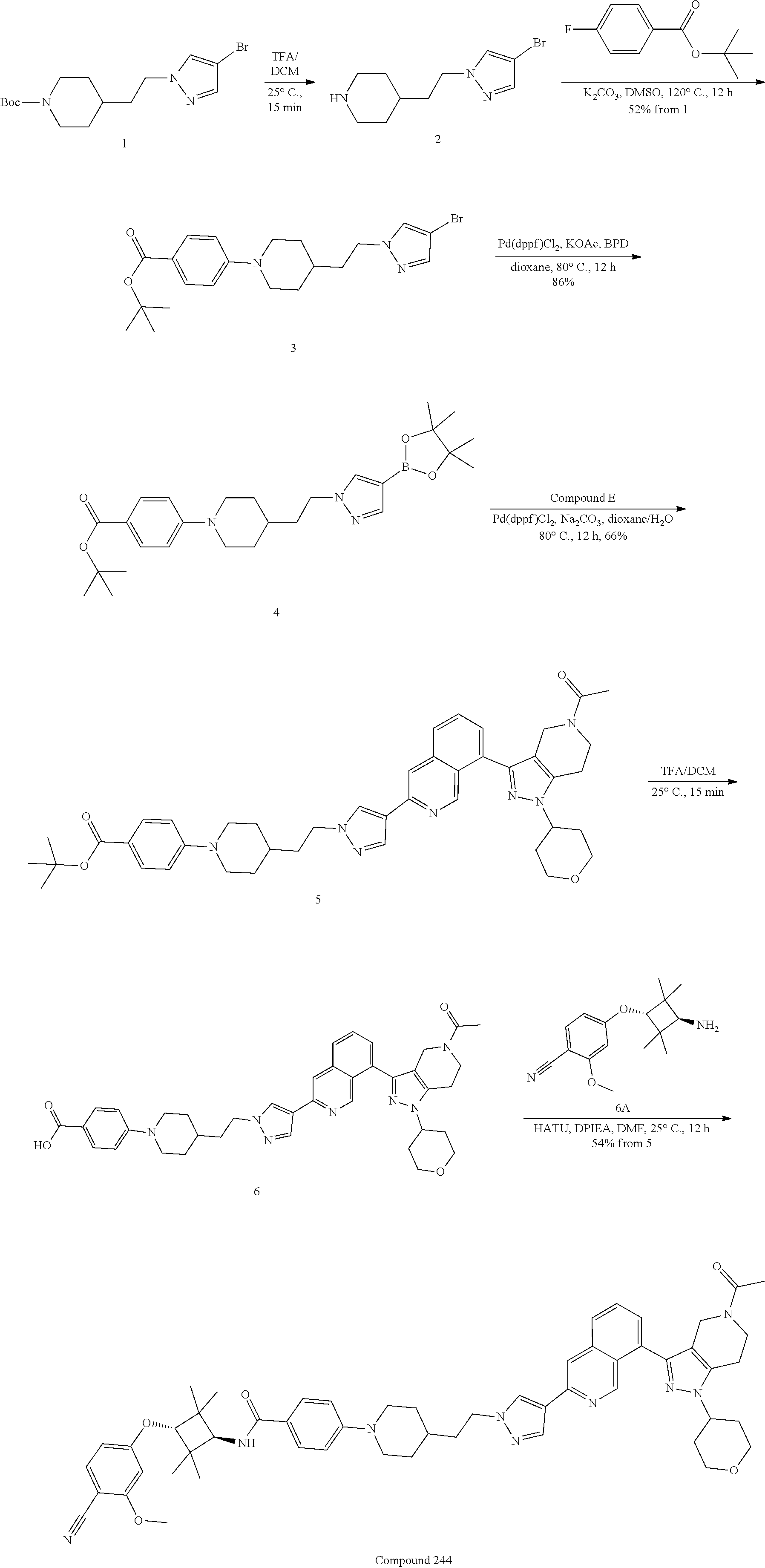

To a solution of tert-butyl 4-[2-(4-bromopyrazol-1-yl)ethyl]piperidine-1-carboxylate (3.22 g, 8.99 mmol, 1.0 eq) in DCM (30 mL) was added TFA (1.02 g, 8.99 mmol, 667.61 μL, 1.0 eq). The mixture was stirred at 25° C. for 15 min. The residue was filtered and concentrated under reduced pressure to give a residue. Compound 4-[2-(4-bromopyrazol-1-yl)ethyl]piperidine (3.20 g) was obtained as a colorless oil and directly used in the next step without further purification.

LC-MS: MS (ESI: t_{R}=0.365 min, m/z=258.1 [M+H⁺]

To a solution of 4-[2-(4-bromopyrazol-1-yl)ethyl]piperidine (3.20 g), tert-butyl 4-fluorobenzoate (1.69 g, 8.60 mmol, 1.0 eq) in DMSO (30 mL) was added K₂CO₃ (11.88 g, 85.98 mmol, 10.0 eq). The mixture was stirred at 120° C. for 12 h. The residue was diluted with water 500 mL and extracted with ethyl acetate 600 mL. The combined organic layers were washed with brine 400 mL, dried over sodium sulfate, filtered and concentrated under reduced pressure to give a residue. The residue was purified by column chromatography (SiO₂, Petroleum ether/Ethyl acetate=20/1 to 5/1). Compound tert-butyl 4-[4-[2-(4-bromopyrazol-1-yl)ethyl]-1-piperidyl]benzoate (1.95 g, 4.49 mmol, 52% yield over two steps) was obtained as a white solid.

¹H NMR (400 MHz, CDCl₃) δ=7.86 (d, 2H, J=8.8 Hz), 7.44 (d, 2H, J=19.6 Hz), 6.85 (d, 2H, J=8.8 Hz), 4.18 (t, 2H, J=7.2 Hz), 3.84 (d, 2H, J=12.8 Hz), 2.80 (t, 2H, J=11.4 Hz), 1.7-2.0 (m, 4H), 1.6-1.6 (m, 9H), 1.4-1.5 (m, 1H), 1.3-1.4 (m, 2H)

LC-MS: MS (ESI: t_{R}=0.614 min, m/z=434.1 [M+H⁺]

To a solution of tert-butyl 4-[4-[2-(4-bromopyrazol-1-yl)ethyl]-1-piperidyl]benzoate (900 mg, 2.07 mmol, 1.0 eq) in dioxane (10 mL) was added BPD (789 mg, 3.11 mmol, 1.5 eq), Pd(dppf)Cl₂ (152 mg, 207.20 μmol, 0.1 eq) and KOAc (610 mg, 6.22 mmol, 3.0 eq). The mixture was stirred at 80° C. for 12 h. The residue was diluted with water 500 mL and extracted with ethyl acetate 600 mL. The combined organic layers were washed with brine 400 mL, dried over sodium sulfate, filtered and concentrated under reduced pressure to give a residue. The residue was purified by column chromatography (SiO₂, Petroleum ether/Ethyl acetate=20/1 to 4/1). Compound tert-butyl 4-[4-[2-[4-(4,4,5,5-tetramethyl-1,3,2-dioxaborolan-2-yl)pyrazol-1-yl]ethyl]-1-piperidyl]benzoate (858 mg, 1.78 mmol, 86% yield) was obtained as a colorless oil.

LC-MS: MS (ESI⁺: t_{R}=0.626 min, m/z=482.3 [M+H⁺]

To a solution of tert-butyl 4-[4-[2-[4-(4,4,5,5-tetramethyl-1,3,2-dioxaborolan-2-yl)pyrazol-1-yl]ethyl]-1-piperidyl]benzoate (138 mg, 285.98 μmol, 1.5 eq), [8-(5-acetyl-1-tetrahydropyran-4-yl-6,7-dihydro-4H-pyrazolo[4,3-c]pyridin-3-yl)-3-isoquinolyl]trifluoromethanesulfonate (100 mg, 190.65 μmol, 1.0 eq) in dioxane (5 mL), H₂O (1 mL) was added Pd(dppf)Cl₂ (7 mg, 9.53 μmol, 0.05 eq) and Na₂CO₃ (61 mg, 571.96 μmol, 3.0 eq). The mixture was stirred at 80° C. for 12 h. The residue was diluted with water 500 mL and extracted with ethyl acetate 600 mL. The combined organic layers were washed with brine 400 mL, dried over sodium sulfate, filtered and concentrated under reduced pressure to give a residue. The residue was purified by prep-TLC (SiO₂, DCM:MeOH=10:1). Compound tert-butyl 4-[4-[2-[4-[8-(5-acetyl-1-tetrahydropyran-4-yl-6,7-dihydro-4H-pyrazolo[4,3-c]pyridin-3-yl)-3-isoquinolyl]pyrazol-1-yl]ethyl]-1-piperidyl]benzoate (92 mg, 126.04 μmol, 66% yield) was obtained as a colorless oil.

LC-MS: MS (ESI⁺): t_{R}=0.547 min, m/z=730.5 [M+H⁺]

To a solution of tert-butyl 4-[4-[2-[4-[8-(5-acetyl-1-tetrahydropyran-4-yl-6,7-dihydro-4H-pyrazolo[4,3-c]pyridin-3-yl)-3-isoquinolyl]pyrazol-1-yl]ethyl]-1-piperidyl]benzoate (152 mg, 208.25 μmol, 1.0 eq) in DCM (3 mL) was added TFA (237 mg, 2.08 mmol, 154.69 μL, 10.0 eq). The mixture was stirred at 25° C. for 15 min. The residue was filtered and concentrated under reduced pressure to give a residue. Compound 4-[4-[2-[4-[8-(5-acetyl-1-tetrahydropyran-4-yl-6,7-dihydro-4H-pyrazolo[4,3-c]pyridin-3-yl)-3-isoquinolyl]pyrazol-1-yl]ethyl]-1-piperidyl]benzoic acid (140 mg) was obtained as a colorless oil and directly used in the next step without further purification.

LC-MS: MS (ESI⁺): t_{R}=0.450 min, m/z=674.5 [M+H⁺]

To a solution of 4-[4-[2-[4-[8-(5-acetyl-1-tetrahydropyran-4-yl-6,7-dihydro-4H-pyrazolo[4,3-c]pyridin-3-yl)-3-isoquinolyl]pyrazol-1-yl]ethyl]-1-piperidyl]benzoic acid (140 mg), 4-(3-amino-2,2,4,4-tetramethyl-cyclobutoxy)-2-methoxy-benzonitrile (58 mg, 213.25 μmol, 1.2 eq) in DMF (2 mL) was added HATU (202.71 mg, 533.11 μmol, 3.0 eq) and DIPEA (229 mg, 1.78 mmol, 309.53 μL, 10.0 eq). The mixture was stirred at 25° C. for 12 h. The residue was diluted with water 500 mL and extracted with ethyl acetate 600 mL. The combined organic layers were washed with brine 400 mL, dried over sodium sulfate, filtered and concentrated under reduced pressure to give a residue. The residue was purified by prep-TLC (SiO₂, DCM:MeOH=10:1). Compound 4-[4-[2-[4-[8-(5-acetyl-1-tetrahydropyran-4-yl-6,7-dihydro-4H-pyrazolo[4,3-c]pyridin-3-yl)-3-isoquinolyl]pyrazol-1-yl]ethyl]-1-piperidyl]-N-[3-(4-cyano-3-methoxy-phenoxy)-2,2,4,4-tetramethyl-cyclobutyl]benzamide (95.45 mg, 97.26 μmol, 54% yield over two steps) was obtained as a yellow solid.

¹H NMR (400 MHz, CDCl₃) δ=10.0-9.7 (m, 1H), 8.1-8.2 (m, 2H), 7.92 (d, 1H, J=16.4 Hz), 7.9-7.7 (m, 2H), 7.68 (d, 2H, J=8.0 Hz), 7.6-7.4 (m, 2H), 6.91 (d, 2H, J=8.8 Hz), 6.5-6.3 (m, 2H), 6.12 (d, 1H, J=8.0 Hz), 4.69 (s, 1H), 4.51 (s, 1H), 4.3-4.2 (m, 3H), 4.2-4.1 (m, 3H), 4.1-4.0 (m, 2H), 3.92 (s, 3H), 3.9-3.8 (m, 3H), 3.57 (t, 2H, J=12.0 Hz), 3.0-2.9 (m, 1H), 2.9-2.8 (m, 3H), 2.43 (dq, 3H, J=4.4, 12.4 Hz), 2.22 (s, 2H), 2.09 (s, 2H), 2.0-1.9 (m, 3H), 1.87 (d, 2H, J=12.0 Hz), 1.5-1.4 (m, 2H), 1.25 (d, 12H, J=10.8 Hz)

LC-MS: MS (ESI⁺): t_{R}=2.616 min, m/z=930.6 [M+H⁺]

The compounds below were prepared in a similar manner as described in Example 34.

| | | | LC-MS |
|---|---|---|---|
| Compound | Name | 1H NMR (CDCl3) | [M + H⁺] |
| 249 | 4-[4-[2-[4-[8-(5-acetyl-1- | δ = 9.70 (d, J = 11.6 Hz, 1H), 8.14 (d, J = | 931.7 |
| | tetrahydropyran-4-yl-6,7- | 5.2 Hz, 1H), 8.09 (s, 1H), 7.86-7.74 (m, | |
| | dihydro-4H-pyrazolo[4,3- | 2H), 7.74-7.64 (m, 3H), 7.55-7.42 (m, | |
| | c]pyridin-3-yl)-3- | 2H), 6.90 (d, J = 8.8 Hz, 2H), 6.47 (d, J = | |
| | isoquinolyl]pyrazol-1- | 2.0 Hz, 1H), 6.40 (dd, J = 2.0, 8.8 Hz, | |
| | yl]ethyl]piperazin-1-yl]-N-[3- | 1H), 6.12 (dd, J = 3.6, 8.0 Hz, 1H), 4.69- | |
| | (4-cyano-3-methoxy-phenoxy)- | 4.46 (m, 2H), 4.37 (t, J = 6.4 Hz, 2H), | |
| | 2,2,4,4-tetramethyl- | 4.32-4.22 (m, 1H), 4.20-4.11 (m, 3H), | |
| | cyclobutyl]benzamide | 4.06-3.98 (m, 2H), 3.92 (s, 3H), 3.84 (t, | |
| | | J = 6.0 Hz, 1H), 3.57 (t, J = 11.6 Hz, | |
| | | 2H), 3.38-3.25 (m, 4H), 3.05-2.89 (m, | |
| | | 3H), 2.86 (t, J = 5.6 Hz, 1H), 2.68 (s, | |
| | | 4H), 2.52-2.37 (m, 2H), 2.23-2.05 (m, | |
| | | 3H), 1.96 (d, J = 12.8 Hz, 2H), 1.25 (d, | |
| | | J = 9.2 Hz, 12H) | |
| 245 | 4-[4-[2-[5-[8-(5-acetyl-1- | δ = 9.83 (d, J = 6.0 Hz, 1H), 9.24 (s, | 941.6 |
| | tetrahydropyran-4-yl-6,7- | 1H), 8.46-8.29 (m, 1H), 8.10 (d, J = | |
| | dihydro-4H-pyrazolo[4,3- | 10.8 Hz, 1H), 7.98-7.85 (m, 1H), 7.76 | |
| | c]pyridin-3-yl)-3-isoquinolyl]- | (td, J = 7.6, 11.7 Hz, 1H), 7.68 (d, J = | |
| | 2-pyridyl]ethyl]-1-piperidyl]-N- | 8.4 Hz, 2H), 7.58 (dd, J = 6.8, 16.2 Hz, | |
| | [3-(4-cyano-3-methoxy- | 1H), 7.45 (d, J = 8.6 Hz, 1H), 7.32 (dd, | |
| | phenoxy)-2,2,4,4-tetramethyl- | J = 4.0, 7.8 Hz, 1H), 6.92 (d, J = 8.4 Hz, | |
| | cyclobutyl]benzamide | 2H), 6.54-6.34 (m, 2H), 6.12 (d, J = 8.0 | |
| | | Hz, 1H), 4.67 (s, 1H), 4.50 (s, 1H), 4.37- | |
| | | 4.22 (m, 1H), 4.22-4.11 (m, 3H), 4.07- | |
| | | 4.00 (m, 2H), 3.96-3.81 (m, 6H), 3.58 ( | |
| | | t, J = 12.0 Hz, 2H), 2.93 (d, J = 6.4 Hz, | |
| | | 2H), 2.90-2.79 (m, 3H), 2.52-2.37 (m, | |
| | | 2H), 2.24-2.06 (m, 3H), 1.97 (d, J = | |
| | | 12.0 Hz, 2H), 1.90 (d, J = 12.0 Hz, 2H), | |
| | | 1.79 (d, J = 7.6 Hz, 3H), 1.57 (s, 1H), | |
| | | 1.46-1.37 (m, 2H), 1.25 (d, J = 10.8 | |
| | | Hz, 12H) | |
| 246 | 4-[4-[2-[5-[1-(5-acetyl-1- | δ = 9.00 (s, 1H), 8.30-8.10 (m, 1H), 7.94 | 946.7 |
| | tetrahydropyran-4-yl-6,7- | (s, 1H), 7.68 (d, 2H, J = 8.6 Hz), 7.50-7.40 | |
| | dihydro-4H-pyrazolo[4,3- | (m, 2H), 6.91 (d, 2H, J = 8.8 Hz), 6.50- | |
| | c]pyridin-3-yl)-3,4-dihydro-2H- | 6.30 (m, 2H), 6.12 (d, 1H, J = 8.0 Hz), | |
| | 1,7-naphthyridin-6-yl]-2- | 4.35 (s, 1H), 4.20-4.10 (m, 5H), 4.05 (s, | |
| | pyridyl]ethyl]-1-piperidyl]-N- | 1H), 3.92 (s, 4H), 3.84 (d, 2H, J = 12.8 | |
| | [3-(4-cyano-3-methoxy- | Hz), 3.80-3.70 (m, 3H), 3.52 (t, 2H, | |
| | phenoxy)-2,2,4,4-tetramethyl- | J = 12.0 Hz), 2.93 (d, 4H, J = 6.4 Hz), 2.90- | |
| | cyclobutyl]benzamide | 2.70 (m, 4H), 2.40-2.20 (m, 2H), 2.20- | |
| | | 2.10 (m, 5H), 1.87 (d, 5H, J = 8.4 Hz), | |
| | | 1.80-1.70 (m, 2H), 1.40-1.30 (m, 2H), | |
| | | 1.25 (d, 12H, J = 11.2 Hz) | |
| 247 | 4-[4-[2-[5-[1-(5-acetyl-1- | δ = 8.51 (d, 1H, J = 1.6 Hz), 7.69 (d, 2H, | 995.7 |
| | tetrahydropyran-4-yl-6,7- | J = 8.8 Hz), 7.57 (dd, 1H, J = 2.4, 7.8 Hz), | |
| | dihydro-4H-pyrazolo[4,3- | 7.46 (d, 1H, J = 8.4 Hz), 7.20-7.10 (m, | |
| | c]pyridin-3-yl)-7- | 1H), 7.10-6.90 (m, 4H), 6.60-6.20 (m, | |
| | (difluoromethyl)-3,4-dihydro- | 3H), 6.12 (d, 1H, J = 8.0 Hz), 4.29 (s, 1H), | |
| | 2H-quinolin-6-yl]-2- | 4.20-4.10 (m, 5H), 4.05 (s, 1H), 3.92 (s, | |
| | pyridyl]ethyl]-1-piperidyl]-N- | 4H), 3.86 (d, 2H, J = 12.4 Hz), 3.80-3.70 | |
| | [3-(4-cyano-3-methoxy- | (m, 3H), 3.53 (t, 2H, J = 12.0 Hz), 2.90- | |
| | phenoxy)-2,2,4,4-tetramethyl- | 2.70 (m, 8H), 2.40-2.20 (m, 2H), 2.20- | |
| | cyclobutyl]benzamide | 2.00 (m, 4H), 1.90 (t, 4H, J = 9.6 Hz), | |
| | | 1.80-1.70 (m, 2H), 1.60-1.50 (m, 2H), | |
| | | 1.50-1.40 (m, 2H), 1.25 (d, 12H, J = 10.4 | |
| | | Hz) | |
| 248 | 4-[4-[2-[4-[1-(5-acetyl-1- | δ = 7.68 (s, 2H), 7.46 (d, J = 8.4 Hz, 1H), | 994.2 |
| | tetrahydropyran-4-yl-6,7- | 7.26-7.23 (m, 3H), 7.23 (s, 1H), 7.00 (d, | |
| | dihydro-4H-pyrazolo[4,3- | J = 20.4 Hz, 1H), 6.93 (d, J = 6.8 Hz, | |
| | c]pyridin-3-yl)-7- | 2H), 6.90 (s, 1H), 6.65-6.29 (m, 3H), | |
| | (difluoromethyl)-3,4-dihydro- | 6.12 (d, J = 8.0 Hz, 1H), 4.34-4.23 (m, | |
| | 2H-quinolin-6-yl]phenyl]ethyl]- | 1H), 4.18-4.10 (m, 5H), 4.05 (s, 1H), | |
| | 1-piperidyl]-N-[3-(4-cyano-3- | 3.93 (s, 4H), 3.86 (d, J = 12.4 Hz, 2H), | |
| | methoxy-phenoxy)-2,2,4,4- | 3.80-3.70 (m, 3H), 3.53 (s, 2H), 2.94- | |
| | tetramethyl- | 2.80 (m, 5H), 2.77 (t, J = 5.6 Hz, 1H), | |
| | cyclobutyl]benzamide | 2.74-2.68 (m, 2H), 2.39-2.25 (m, 2H), | |
| | | 2.18 (s, 1H), 2.14-2.03 (m, 4H), 1.89 ( | |
| | | d, J = 12.4 Hz, 4H), 1.72-1.49 (m, 7H), | |
| | | 1.46-1.36 (m, 2H), 1.25 (d, J = 10.8 Hz, | |
| | | 12H) | |
| 250 | 4-[4-[2-[5-[8-(5-acetyl-1- | δ = 9.84 (d, J = 6.4 Hz, 1H), 9.25 (t, J = | 942.6 |
| | tetrahydropyran-4-yl-6,7- | 2.8 Hz, 1H), 8.39 (dt, J = 2.4, 7.6 Hz, | |
| | dihydro-4H-pyrazolo[4,3- | 1H), 8.10 (d, J = 10.8 Hz, 1H), 7.91 (dd, | |
| | c]pyridin-3-yl)-3-isoquinolyl]- | J = 8.4, 14.4 Hz, 1H), 7.81-7.68 (m, | |
| | 2-pyridyl]ethyl] piperazin-1-yl]- | 3H), 7.59 (dd, J = 6.8, 16.4 Hz, 1H), 7.46 | |
| | N-[3-(4-cyano-3-methoxy- | (d, J = 8.4 Hz, 1H), 7.37 (dd, J = 4.4, 8.0 | |
| | phenoxy)-2,2,4,4-tetramethyl- | Hz, 1H), 6.93 (d, J = 8.8 Hz, 2H), 6.47 | |
| | cyclobutyl]benzamide | (d, J = 2.0 Hz, 1H), 6.40 (dd, J = 2.4, 8.8 | |
| | | Hz, 1H), 6.13 (d, J = 8.4 Hz, 1H), 4.67 | |
| | | (s, 1H), 4.51 (s, 1H), 4.35-4.25 (m, 1H), | |
| | | 4.21-4.13 (m, 3H), 4.06-4.00 (m, 2H), | |
| | | 3.92 (s, 3H), 3.86 (t, J = 5.6 Hz, 1H), | |
| | | 3.58 (t, J = 12.0 Hz, 2H), 3.37 (s, 4H), | |
| | | 3.20-3.11 (m, 2H), 2.94 (t, J = 5.6 Hz, | |
| | | 3H), 2.88 (t, J = 5.6 Hz, 1H), 2.77 (s, | |
| | | 4H), 2.46-2.45 (m, 2H), 2.22 (s, 1H), | |
| | | 2.09 (s, 1H), 2.01-1.95 (m, 2H), 1.25 (d, | |
| | | J = 10.8 Hz, 12H) | |
| 251 | 4-[4-[2-[5-[1-(5-acetyl-1- | 8 = 9.00 (dd, J = 2.0, 5.6 Hz, 1H), 8. 12 (dd, | 947.7 |
| | tetrahydropyran-4-yl-6,7- | J = 8.4, 12.0 Hz, 1H), 7.93 (s, 1H), 7.70 | |
| | dihydro-4H-pyrazolo[4,3- | (d, J = 8.4 Hz, 2H), 7.3-7.5 (m, 2H), 6.92 | |
| | c]pyridin-3-yl)-3,4-dihydro-2H- | (d, J = 8.8 Hz, 2H), 6.4-6.5 (m, 2H), 6.12 | |
| | 1,7-naphthyridin-6-yl]-2- | (d, J = 8.0 Hz, 1H), 4.35 (s, 1H), 4.1-4.2 | |
| | pyridyl]ethyl]piperazin-1-yl]-N- | (m, 5H), 4.05 (s, 1H), 3.9-4.0 (m, 4H), | |
| | [3-(4-cyano-3-methoxy- | 3.74 (qd, J = 5.6, 11.6 Hz, 3H), 3.52 (t, | |
| | phenoxy)-2,2,4,4-tetramethyl- | J = 12.0 Hz, 2H), 3.34 (s, 4H), 3.0-3.1 (m, | |
| | cyclobutyl]benzamide | 2H), 2.8-3.0 (m, 5H), 2.6-2.8 (m, 5H), | |
| | | 2.2-2.4 (m, 2H), 2.0-2.2 (m, 4H), 1.86 (d, | |
| | | J = 12.4 Hz, 2H), 1.25 (d, J = 10.2 Hz, | |
| | | 12H) | |
| 254 | 4-[4-[2-[4-[8-(5-acetyl-1- | δ = 10.26-9.95 (m, 1H), 8.28-8.14 (m, | 940.2 |
| | tetrahydropyran-4-yl-6,7- | 1H), 8.06-7.92 (m, 4H), 7.91-7.75 (m, | |
| | dihydro-4H-pyrazolo[4,3- | 1H), 7.69 (d, J = 8.8 Hz, 2H), 7.64-7.33 | |
| | c]pyridin-3-yl)-3- | (m, 4H), 6.97-6.89 (m, 2H), 6.47 (d, J = | |
| | isoquinolyl]phenyl]ethyl]-1- | 2.0 Hz, 1H), 6.38 (s, 1H), 6.16-6.10 (m, | |
| | piperidyl]-N-[3-(4-cyano-3- | 1H), 4.78-4.50 (m, 2H), 4.42-4.25 (m, | |
| | methoxy-phenoxy)-2,2,4,4- | 1H), 4.20-4.13 (m, 3H), 4.08-3.99 (m, | |
| | tetramethyl- | 2H), 3.92 (s, 3H), 3.89-3.83 (m, 3H), | |
| | cyclobutyl]benzamide | 3.63-3.54 (m, 2H), 2.98-2.94 (m, 1H), | |
| | | 2.89-2.84 (m, 2H), 2.82-2.76 (m, 2H), | |
| | | 2.47-2.38 (m, 2H), 2.08 (s, 4H), 2.07- | |
| | | 1.99 (m, 2H), 1.91-1.86 (m, 2H), 1.72- | |
| | | 1.66 (m, 2H), 1.43-1.36 (m, 2H), 1.27 | |
| | | (s, 6H), 1.24 (s, 6H) | |

### Example 35: Synthesis of 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carbonyl]-1-piperidyl]-N-[3-(4-cyano-3-methoxy-phenoxy)-2,2,4,4-tetramethyl-cyclobutyl]benzamide (Compound 252)

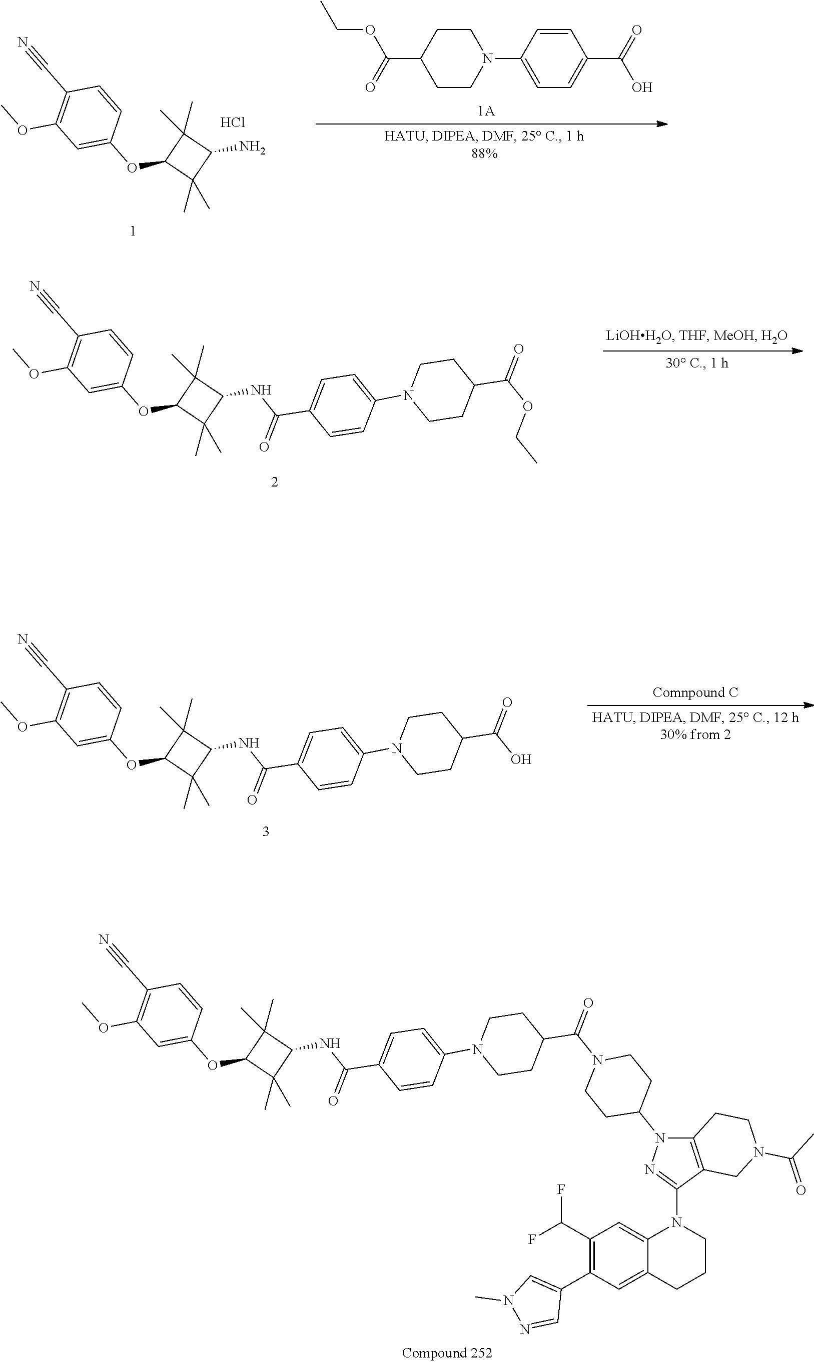

Synthesis of 1 was reported in WO2021/127443 A1.

Synthesis of 1A was reported in WO2023/242598 A1

To a solution of 4-(3-amino-2,2,4,4-tetramethyl-cyclobutoxy)-2-methoxy-benzonitrile (400 mg) and 4-(4-ethoxycarbonyl-1-piperidyl) benzoic acid (356 mg, 1.29 mmol, 1.0 eq) in DMF (4 mL) was added HATU (587 mg, 1.54 mmol, 1.2 eq) and DIPEA (831 mg, 6.43 mmol, 1.1 mL, 5.0 eq). The mixture was stirred at 25° C. for 1 h. Then the reaction mixture was diluted with water (40 mL) and extracted with EtOAc (40 mL×2). The combined organic layers were washed with brine (60 ml×2), dried over Na₂SO₄, filtered and concentrated under reduced pressure to give a residue. The residue was purified by column chromatography (SiO₂, Petroleum ether/Ethyl acetate=10/1 to 1/1). Compound ethyl 1-[4-[[3-(4-cyano-3-methoxy-phenoxy)-2,2,4,4-tetramethyl-cyclobutyl]carbamoyl]phenyl]piperidine-4-carboxylate (605 mg, 1.13 mmol, 88% yield) was obtained as a white oil.

LC-MS: MS (ESI⁺): t_{R}=0.896 min, m/z=534.4 [M+H⁺]

To a solution of ethyl 1-[4-[[3-(4-cyano-3-methoxy-phenoxy)-2,2,4,4-tetramethyl-cyclobutyl]carbamoyl]phenyl]piperidine-4-carboxylate (605 mg, 1.13 mmol, 1.0 eq) in THF (4 mL), MeOH (2 mL) and H₂O (2 mL) was added LiOH H₂O (142 mg, 3.40 mmol, 3.0 eq). The mixture was stirred at 30° C. for 1 h. The mixture was concentrated to give a residue. Compound 1-[4-[[3-(4-cyano-3-methoxy-phenoxy)-2,2,4,4-tetramethyl-cyclobutyl]carbamoyl]phenyl]piperidine-4-carboxylic acid (508 mg) was obtained as a white solid and directly used into the next step without further purification.

LC-MS: MS (ESI⁺): t_{R}=0.567 min, m/z=506.3 [M+H⁺]

To a solution of 1-[4-[[3-(4-cyano-3-methoxy-phenoxy)-2,2,4,4-tetramethyl-cyclobutyl]carbamoyl]phenyl]piperidine-4-carboxylic acid (76 mg) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (70 mg, 137 μmol, 1.0 eq) in DMF (1.5 mL) was added HATU (62 mg, 164 μmol, 1.2 eq) and DIPEA (88 mg, 686 μmol, 119 μL, 5.0 eq). The mixture was stirred at 25° C. for 12 h. The mixture was filtered and concentrated to give a residue, the residue was purified by prep-HPLC (column: Phenomenex luna C18 150*25 mm*10 um; mobile phase: [water (FA)-ACN]; gradient: 55%-85% B over 10 min). Compound 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carbonyl]-1-piperidyl]-N-[3-(4-cyano-3-methoxy-phenoxy)-2,2,4,4-tetramethyl-cyclobutyl]benzamide (43.92 mg, 30% yield over two steps) was obtained as a white solid.

¹H NMR (400 MHz, DMSO-d₆) δ=7.80-7.70 (m, 3H), 7.69-7.60 (m, 1H), 7.58-7.45 (m, 2H), 7.10 (s, 1H), 7.02-6.93 (m, 2H), 6.92 (s, 3H), 6.57-6.48 (m, 1H), 4.58-4.42 (m, 1H), 4.42-4.31 (m, 1H), 4.26 (s, 1H), 4.21-4.08 (m, 3H), 4.08-4.01 (m, 1H), 3.83 (s, 8H), 3.78-3.68 (m, 2H), 3.61-3.55 (m, 2H), 3.28-3.17 (m, 1H), 2.96-2.81 (m, 6H), 2.80-2.65 (m, 2H), 2.08 (s, 2H), 2.01-1.86 (m, 6H), 1.79-1.58 (m, 5H), 1.22 (s, 6H), 1.14 (s, 6H).

LC-MS: MS (ESI⁺): t_{R}=2.611 min, m/z=997.2 [M+H⁺]

### Example 36: Synthesis of 4-[4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-1-piperidyl]-N-[3-(4-cyano-3-methoxy-phenoxy)-2,2,4,4-tetramethyl-cyclobutyl]benzamide (Compound 253)

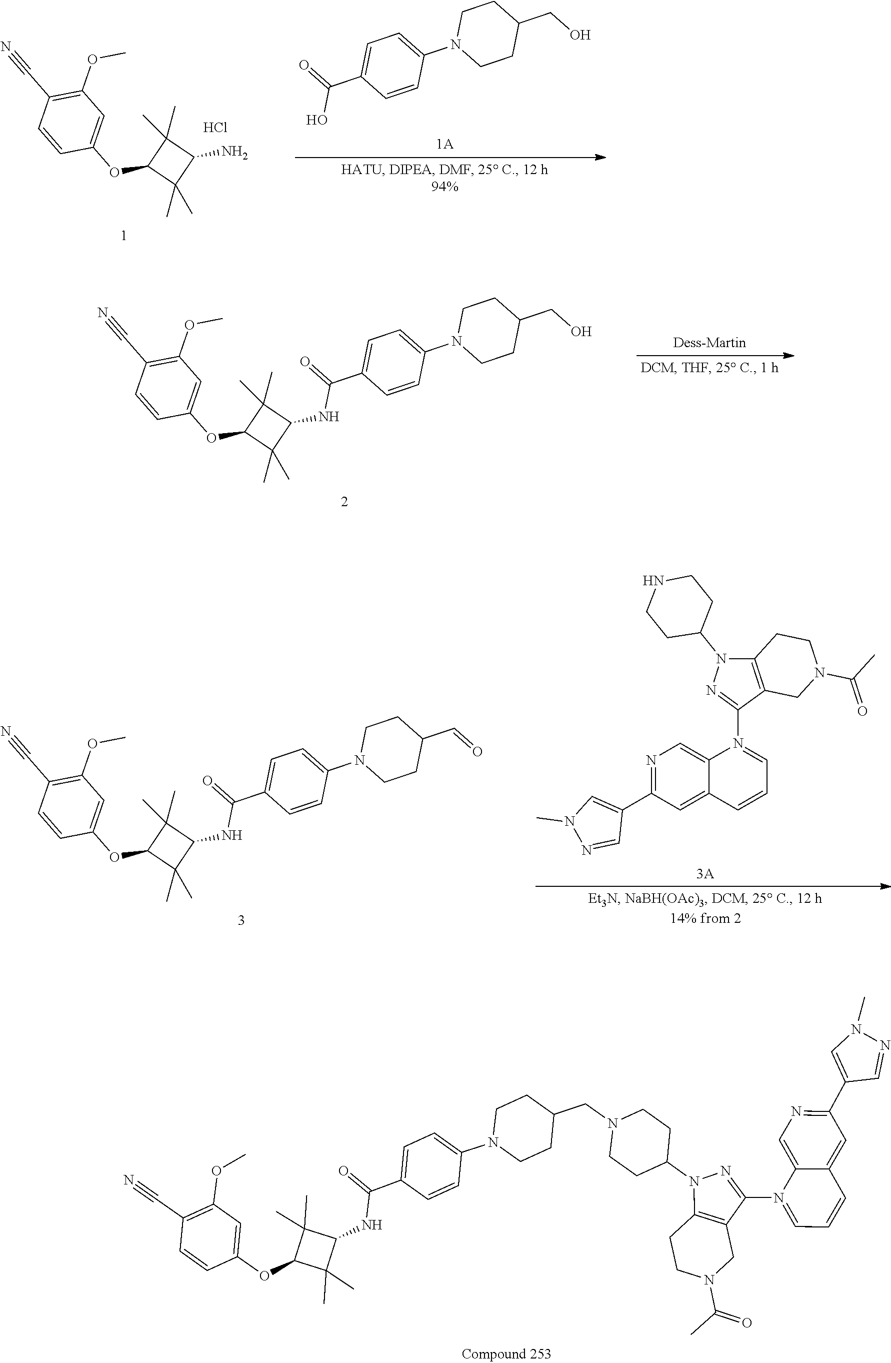

Synthesis of 1A was reported in Journal of Medicinal Chemistry, 2021, vol. 64, #17, p. 12831-12854.

To a solution of 4-(3-amino-2,2,4,4-tetramethyl-cyclobutoxy)-2-methoxy-benzonitrile (200 mg, 643 μmol, 1.0 eq) and 4-[4-(hydroxymethyl)-1-piperidyl]benzoic acid (151 mg, 643 μmol, 1.0 eq) in DMF (3 mL) was added HATU (293 mg, 772 μmol, 1.2 eq) and DIPEA (415 mg, 3.22 mmol, 560 μL, 5.0 eq). The mixture was stirred at 25° C. for 12 h. Then reaction mixture was diluted with water (30 mL) and extracted with EtOAc (30 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=30/1 to 0/1). Compound N-[3-(4-cyano-3-methoxy-phenoxy)-2,2,4,4-tetramethyl-cyclobutyl]-4-[4-(hydroxymethyl)-1-piperidyl]benzamide (300 mg, 610 μmol, 94% yield) was obtained as a white solid.

LC-MS: MS (ESI⁺) t_{R}=0.533 min, m/z=492.3 [M+H⁺]

To a solution of N-[3-(4-cyano-3-methoxy-phenoxy)-2,2,4,4-tetramethyl-cyclobutyl]-4-[4-(hydroxymethyl)-1-piperidyl]benzamide (300 mg, 610 μmol, 1.0 eq) in DCM (5 mL) and THF (0.5 mL) was added Dess-Martin (388 mg, 915 μmol, 283 μL, 1.5 eq). The mixture was stirred at 25° C. for 1 h. The reaction mixture was diluted with water (15 mL) and extracted with DCM (15 mL×2). The combined organic layers were washed with brine (20 mL×2), dried over Na₂SO₄, filtered and concentrated under reduced pressure to give a residue. Compound N-[3-(4-cyano-3-methoxy-phenoxy)-2,2,4,4-tetramethyl-cyclobutyl]-4-(4-formyl-1-piperidyl) benzamide (298 mg) was obtained as a red oil and directly used into the next step without further purification.

LC-MS: MS (ESI⁺): t_{R}=0.582 min, m/z=490.3 [M+H⁺]

To a solution of 1-[3-[3-(1-methylpyrazol-4-yl)-8-isoquinolyl]-1-(4-piperidyl)-6,7-dihydro-4H-pyrazolo[4,3-c]pyridin-5-yl]ethanone (81 mg, 177 μmol, 1.0 eq) and N-[3-(4-cyano-3-methoxy-phenoxy)-2,2,4,4-tetramethyl-cyclobutyl]-4-(4-formyl-1-piperidyl)benzamide (95 mg) in DCM (2 mL) was added Et₃N (89 mg, 889 μmol, 123 μL, 5.0 eq) and NaBH(OAc)₃ (113 mg, 533 μmol, 3.0 eq). The mixture was stirred at 25° C. for 12 h. The mixture was filtered and concentrated to give a residue, the residue was purified by prep-HPLC (column: Waters xbridge 150*25 mm 10 um; mobile phase: [water (NH₄HCO₃)-ACN]; gradient: 55%-75% B over 8 min). Compound 4-[4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-1-piperidyl]-N-[3-(4-cyano-3-methoxy-phenoxy)-2,2,4,4-tetramethyl-cyclobutyl]benzamide (25.25 mg, 14% yield over two steps) was obtained as a white solid.

¹H NMR (400 MHz, DMSO-d₆) δ=9.85 (s, 1H), 8.37 (s, 1H), 8.14-8.03 (m, 2H), 7.92-7.85 (m, 1H), 7.83-7.76 (m, 1H), 7.75-7.68 (m, 2H), 7.67-7.61 (m, 1H), 7.60-7.45 (m, 2H), 7.00-6.90 (m, 2H), 6.67-6.61 (m, 1H), 6.57-6.47 (m, 1H), 4.57 (s, 2H), 4.26 (s, 1H), 4.23-4.12 (m, 1H), 4.10-4.00 (m, 1H), 3.96-3.89 (m, 6H), 3.88-3.77 (m, 4H), 3.04-2.92 (m, 3H), 2.90-2.63 (m, 3H), 2.26-2.18 (m, 2H), 2.18-2.06 (m, 6H), 2.06-1.88 (m, 4H), 1.85-1.74 (m, 3H), 1.25-1.10 (m, 13H).

LC-MS: MS (ESI⁺): t_{R}=2.042 min, m/z=929.6 [M+H⁺]

### Example 37: Synthesis of 4-[5-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-2-azabicyclo[2.2.1]heptan-2-yl]-N-[3-(4-cyano-3-methoxy-phenoxy)-2,2,4,4-tetramethyl-cyclobutyl]benzamide (Compound 255)

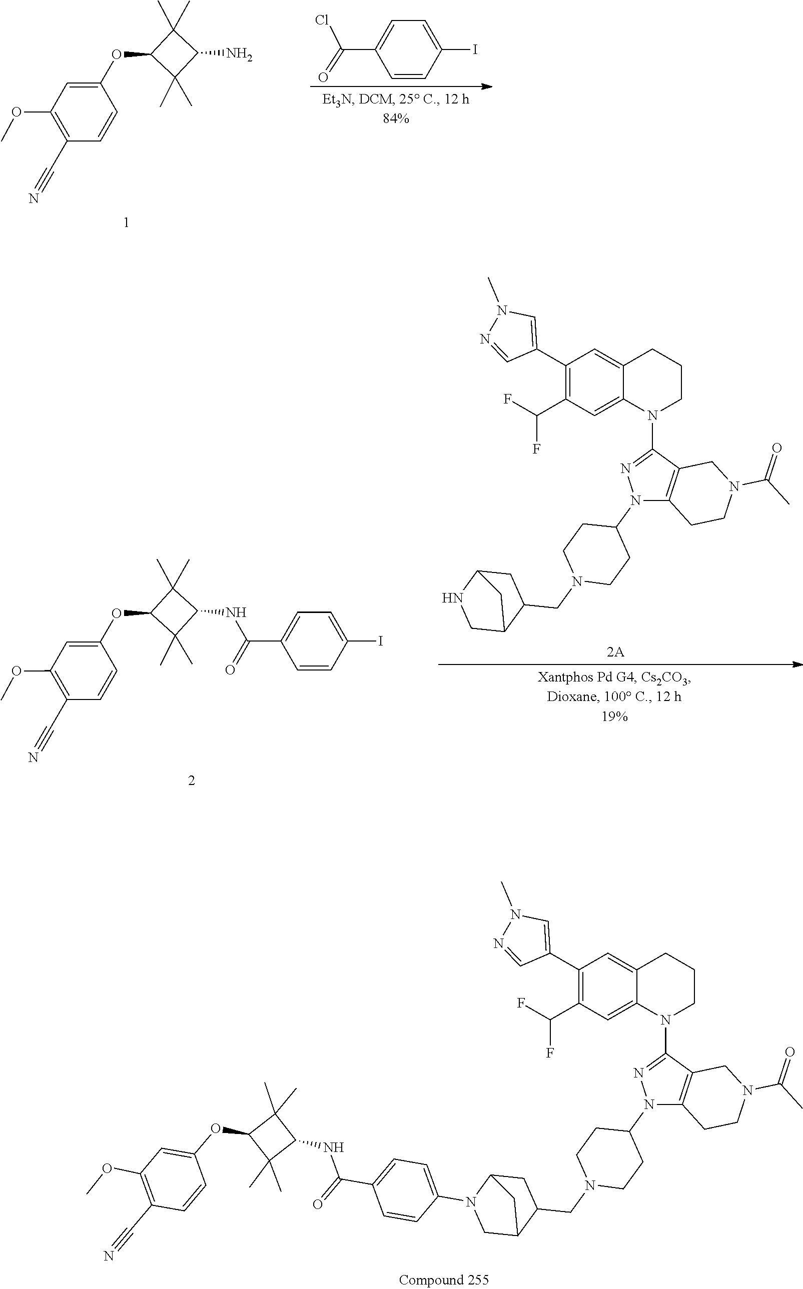 [SMILES: COc1cc(O[C@H]2C(C)(C)[C@H](N)C2(C)C)ccc1C#N ; O=C(Cl)c1ccc(I)cc1 ; COc1cc(O[C@H]2C(C)(C)[C@H](NC(=O)c3ccc(I)cc3)C2(C)C)ccc1C#N ; CC(=O)N1CCc2c(c(N3CCCc4cc(-c5cnn(C)c5)c(C(F)F)cc43)nn2C2CCN(CC3CC4CC3CN4)CC2)C1 ; COc1cc(O[C@H]2C(C)(C)[C@H](NC(=O)c3ccc(N4CC5CC4CC5CN4CCC(n5nc(N6CCCc7cc(-c8cnn(C)c8)c(C(F)F)cc76)c6c5CCN(C(C)=O)C6)CC4)cc3)C2(C)C)ccc1C#N]

To a solution of 4-(3-amino-2,2,4,4-tetramethyl-cyclobutoxy)-2-methoxy-benzonitrile (100 mg, 322 μmol, 1.0 eq), 4-iodobenzoyl chloride (130 mg, 488 μmol, 1.5 eq) in DCM (1 mL) was added Et₃N (146 mg, 1.44 mmol, 0.2 mL, 4.5 eq). The mixture was stirred at 25° C. for 12 h. The reaction mixture was concentrated to give the residue. The residue was purified by prep-TLC (SiO₂, Petroleum ether/Ethyl acetate=1/1) to give N-[3-(4-cyano-3-methoxy-phenoxy)-2,2,4,4-tetramethyl-cyclobutyl]-4-iodo-benzamide (144 mg, 273 μmol, 84% yield) as a white solid.

LC-MS: MS (ESI⁺): t_{R}=0.656 min, m/z=505.2 [M+H⁺]

A mixture of N-[3-(4-cyano-3-methoxy-phenoxy)-2,2,4,4-tetramethyl-cyclobutyl]-4-iodo-benzamide (66 mg, 131 μmol, 1.2 eq), 1-[1-[1-(2-azabicyclo[2.2.1]heptan-5-ylmethyl)-4-piperidyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (66 mg, 107 μmol, 1.0 eq), Xantphos Pd G4 (14 mg, 15 μmol, 0.1 eq) and Cs₂CO₃ (165 mg, 507 μmol, 4.8 eq) in dioxane (1 mL) was degassed and purged with N₂ for 3 times, and then the mixture was stirred at 100° C. for 12 h under N₂ atmosphere. The reaction mixture was filtered and concentrated under reduced pressure to give a residue. The residue was purified by prep-HPLC (column: Phenomenex luna C18 150*25 mm*10 um; mobile phase: [water(FA)-ACN]; gradient: 31%-61% B over 10 min) to give 4-[5-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-2-azabicyclo[2.2.1]heptan-2-yl]-N-[3-(4-cyano-3-methoxy-phenoxy)-2,2,4,4-tetramethyl-cyclobutyl]benzamide (20.25 mg, 21 μmol, 19% yield) as a pink solid.

¹H NMR (400 MHz, DMSO-d₆): δ=7.75 (s, 1H), 7.70 (d, J=8.8 Hz, 2H), 7.64 (d, J=8.4 Hz, 1H), 7.50 (s, 1H), 7.40-7.32 (m, 1H), 7.10 (s, 1H), 6.93-6.66 (m, 2H), 6.63 (d, J=2.0 Hz, 1H), 6.53 (t, J=2.8, 5.6 Hz, 3H), 4.26 (s, 1H), 4.24-4.14 (m, 2H), 4.11 (s, 1H), 4.04 (d, J=9.2 Hz, 1H), 4.02-3.95 (m, 1H), 3.90 (s, 3H), 3.86 (s, 3H), 3.75-3.66 (m, 2H), 3.59 (q, J=5.6 Hz, 2H), 3.44 (d, J=5.6 Hz, 1H), 3.04-2.88 (m, 2H), 2.87-2.80 (m, 4H), 2.18-2.08 (m, 2H), 2.07 (s, 3H), 2.02-1.93 (m, 6H), 1.91-1.77 (m, 4H), 1.77-1.70 (m, 1H), 1.69-1.57 (m, 2H), 1.31 (s, 1H), 1.21 (s, 6H), 1.14 (s, 6H), 1.11-1.01 (m, 1H).

LC-MS: MS (ESI⁺): t_{R}=2.037 min, m/z=995.8 [M+H⁺]

SFC: t_{R1=1.235} min, 15%, t_{R2}=1.350 min, 14%, t_{R3}=1.877 min, 31%, t_{R4}=1.913 min, 40%

The compounds below were prepared in a similar manner as described in Example 37.

| | | | LC-MS |
|---|---|---|---|
| Compound | Name | ¹H NMR (CD₃OD) | [M + H⁺] |
| 305 | 4-[4-[4-[5-acetyl-3-[7- | δ 8.49 (s, 3H), 7.76-7.74 (d, J = 8.8 Hz, | 983.6 |
| | (difluoromethyl)-6-(1- | 2H), 7.63 (s, 1H), 7.54-7.52 (d, J = 8.4 | |
| | methylpyrazol-4-yl)-3,4- | Hz, 2H), 7.09 (d, J = 8.4 Hz, 1H), 7.04- | |
| | dihydro-2H-quinolin-1-yl]-6,7- | 7.01 (d, J = 9.2 Hz, 2H), 6.72-6.42 (m, | |
| | dihydro-4H-pyrazolo[4,3- | 4H), 4.31-4.21 (m, 6H), 3.93-3.88 (m, | |
| | c]pyridin-1-yl]-1-piperidyl]-4- | 7H), 3.82 (m, 1H), 3.75-3.65 (m, 4H), | |
| | methyl-1-piperidyl]-N-[3-(4- | 3.55-3.45 (m, 2H), 3.25-3.15 (m, 3H), | |
| | cyano-3-methoxy-phenoxy)- | 2.90-2.75 (m, 5H), 2.18-1.85 (m, 13H), | |
| | 2,2,4,4-tetramethyl- | 1.41-1.24 (m, 15H). | |
| | cyclobutyl]benzamide | | |

### Example 38: Synthesis of 4-[4-[4-[5-acetyl-3-(3-cyclopropyl-8-isoquinolyl)-6,7-dihydro-4H-pyrazolo[4,3-c]pyridin-1-yl]piperidine-1-carbonyl]-1-piperidyl]-N-[3-(4-cyano-3-methoxy-phenoxy)-2,2,4,4-tetramethyl-cyclobutyl]benzamide (Compound 256)

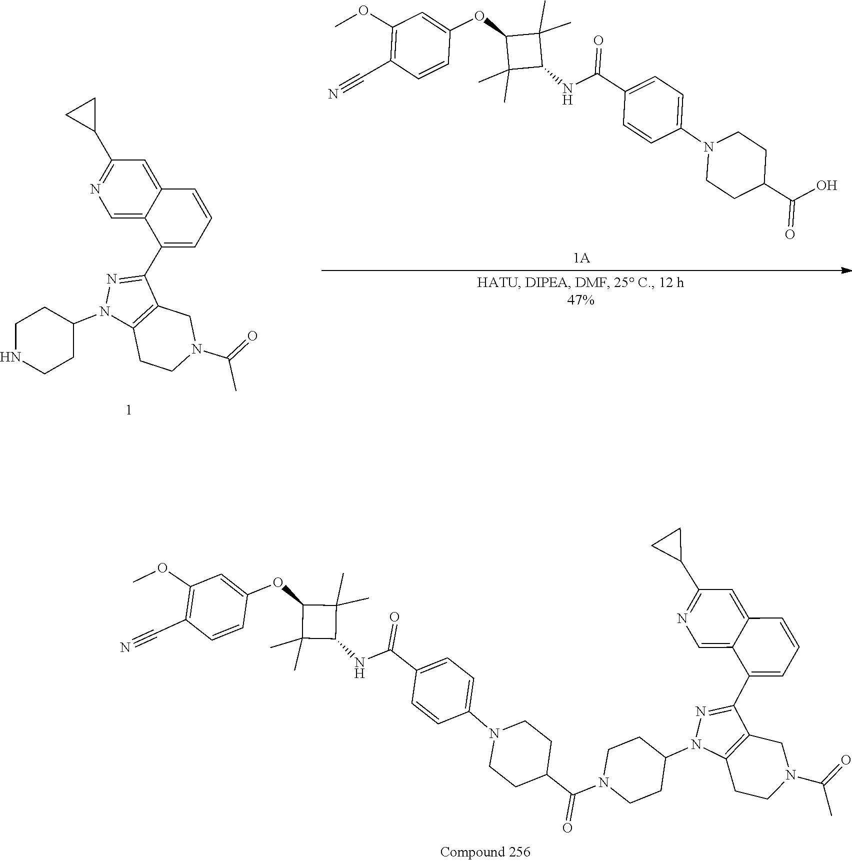 [SMILES: CC(=O)N1CCc2c(c(-c3cccc4cc(C5CC5)ncc34)nn2C2CCNCC2)C1 ; COc1cc(O[C@H]2C(C)(C)[C@H](NC(=O)c3ccc(N4CCC(C(=O)O)CC4)cc3)C2(C)C)ccc1C#N ; COc1cc(O[C@H]2C(C)(C)[C@H](NC(=O)c3ccc(N4CCC(C(=O)N5CCC(n6nc(-c7cccc8cc(C9CC9)ncc78)c7c6CCN(C(C)=O)C7)CC5)CC4)cc3)C2(C)C)ccc1C#N]

To a solution of 1-[3-(3-cyclopropyl-8-isoquinolyl)-1-(4-piperidyl)-6,7-dihydro-4H-pyrazolo[4,3-c]pyridin-5-yl]ethanone (56 mg, 134 μmol, 1.0 eq) and 1-[4-[[3-(4-cyano-3-methoxy-phenoxy)-2,2,4,4-tetramethyl-cyclobutyl]carbamoyl]phenyl]piperidine-4-carboxylic acid (74 mg, 148 μmol, 1.1 eq) in DMF (1 mL) was added HATU (61 mg, 161 μmol, 1.2 eq) and DIPEA (87 mg, 673 μmol, 117 μL, 5.0 eq). The mixture was stirred at 25° C. for 12 h. The mixture was filtered and concentrated to give a residue, the residue was purified by prep-HPLC (column: Phenomenex luna C18 150*25 mm*10 um; mobile phase: [water (FA)-ACN]; gradient: 37%-67% B over 10 min). Compound 4-[4-[4-[5-acetyl-3-(3-cyclopropyl-8-isoquinolyl)-6,7-dihydro-4H-pyrazolo[4,3-c]pyridin-1-yl]piperidine-1-carbonyl]-1-piperidyl]-N-[3-(4-cyano-3-methoxy-phenoxy)-2,2,4,4-tetramethyl-cyclobutyl]benzamide (61.6 mg, 47% yield) was obtained as a yellow solid.

¹H NMR (400 MHz, DMSO-d₆) δ=9.73 (s, 1H), 7.95-7.81 (m, 3H), 7.78-7.69 (m, 2H), 7.68-7.54 (m, 2H), 7.52-7.45 (m, 1H), 7.02-6.89 (m, 2H), 6.71-6.60 (m, 1H), 6.56-6.47 (m, 1H), 4.69-4.42 (m, 4H), 4.31-4.14 (m, 2H), 4.09-4.00 (m, 1H), 3.95-3.78 (m, 7H), 3.29-3.23 (m, 1H), 3.01-2.83 (m, 5H), 2.82-2.71 (m, 1H), 2.32-2.22 (m, 1H), 2.16-1.97 (m, 6H), 1.92-1.78 (m, 1H), 1.76-1.56 (m, 4H), 1.28-1.19 (m, 6H), 1.19-1.11 (m, 6H), 1.09-0.95 (m, 4H).

LC-MS: MS (ESI⁺): t_{R}=2.23 min, m/z=903.4 [M+H⁺]

The compounds below were prepared in a similar manner as described in Example 38.

| | | | LC-MS |
|---|---|---|---|
| Compound | Name | ¹H NMR (DMSO-d₆) | [M + H⁺] |
| 257 | 4-[4-[4-[5-acetyl-3-[6- | δ = 8.82-8.70 (m, 1H), 7.99-7.88 (m, | 952.6 |
| | cyclopropyl-7-(difluoromethyl)- | 1H), 7.79-7.70 (m, 3H), 7.69-7.61 (m, | |
| | 1-naphthyl]-6,7-dihydro-4H- | 2H), 7.61-7.28 (m, 3H), 7.06-6.91 (m, | |
| | pyrazolo[4,3-c]pyridin-1- | 2H), 6.66-6.62 (m, 1H), 6.57-6.51 (m, | |
| | yl] piperidine-1-carbonyl]-1- | 1H), 4.61-4.40 (m, 4H), 4.33-4.14 (m, | |
| | piperidyl]-N-[3-(4-cyano-3- | 2H), 4.09-4.00 (m, 1H), 3.98-3.76 (m, | |
| | methoxy-phenoxy)-2,2,4,4- | 7H), 3.31-3.20 (m, 1H), 3.02-2.75 (m, | |
| | tetramethyl- | 6H), 2.28-2.17 (m, 1H), 2.15-1.97 (m, | |
| | cyclobutyl]benzamide | 6H), 1.95-1.81 (m, 1H), 1.79-1.59 (m, | |
| | | 4H), 1.22 (s, 6H), 1.15 (s, 6H), 1.07- | |
| | | 1.02 (m, 2H), 0.91-0.82 (m, 2H) | |

### Example 39: Synthesis of 4-[2-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-9-azadispiro[3.1.5⁶.1⁴]dodecan-9-yl]-N-[3-(4-cyano-3-methoxy-phenoxy)-2,2,4,4-tetramethyl-cyclobutyl]benzamide (Compound 286)

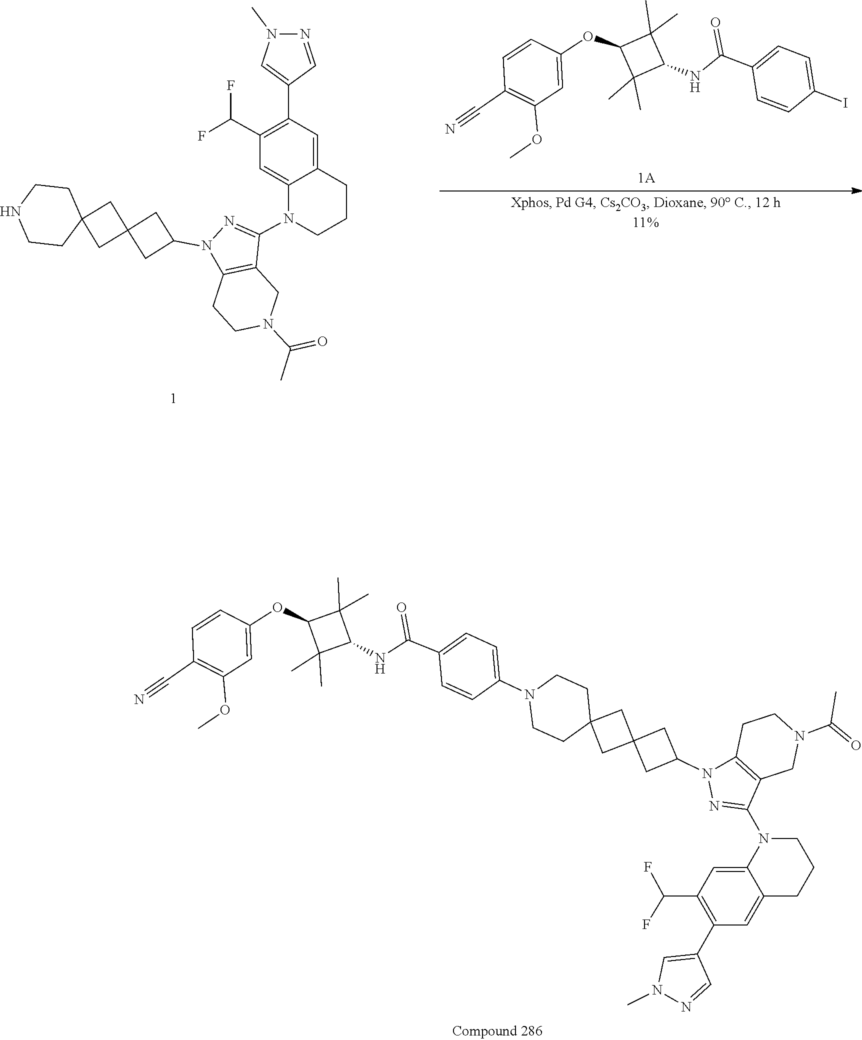 [SMILES: CC(=O)N1CCc2c(c(N3CCCc4cc(-c5cnn(C)c5)c(C(F)F)cc43)nn2C2CC3(C2)CC2(CCNCC2)C3)C1 ; COc1cc(O[C@H]2C(C)(C)[C@H](NC(=O)c3ccc(I)cc3)C2(C)C)ccc1C#N ; COc1cc(O[C@H]2C(C)(C)[C@H](NC(=O)c3ccc(N4CCC5(CC4)CC4(CC(n6nc(N7CCCc8cc(-c9cnn(C)c9)c(C(F)F)cc87)c7c6CCN(C(C)=O)C7)C4)C5)cc3)C2(C)C)ccc1C#N]

A mixture of 1-[1-(9-azadispiro[3.1.5⁶.1⁴]dodecan-2-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (160 mg, 227 mol, 1.0 eq), N-[3-(4-cyano-3-methoxy-phenoxy)-2,2,4,4-tetramethyl-cyclobutyl]-4-iodo-benzamide (114 mg, 227 mol, 1.0 eq), Cs₂CO₃ (222 mg, 682 mol, 3.0 eq), Xphos Pd G4 (19 mg, 22 mol, 0.1 eq) in dioxane (5 mL) was degassed and purged with N₂ for 3 times, and then the mixture was stirred 90° C. for 12 h under N₂ atmosphere. The reaction mixture was diluted with water (15 mL) and extracted with EtOAc (15 mL×2). The combined organic layers were washed with brine (30 mL×2), dried over Na₂SO₄, filtered and concentrated under reduced pressure to give a residue. The residue was purified by prep-HPLC (column: Unisil 3-100 C18 Ultra 150*50 mm*3 um; mobile phase: [water(FA)-ACN]; gradient: 65%-95% B over 10 min). Compound 4-[2-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-9-azadispiro[3.1.5⁶.14]dodecan-9-yl]-N-[3-(4-cyano-3-methoxy-phenoxy)-2,2,4,4-tetramethyl-cyclobutyl]benzamide (24.16 mg, 11% yield) was obtained as a white solid.

¹H NMR (400 MHz, CDCL₃) δ=8.01 (s, 1H), 7.72-7.63 (m, 2H), 7.57-7.49 (m, 1H), 7.48-7.38 (m, 2H), 7.07-6.96 (m, 1H), 6.93-6.86 (m, 3H), 6.69-6.36 (m, 3H), 6.17-6.06 (m, 1H), 4.55-4.43 (m, 1H), 4.25-4.08 (m, 3H), 4.07-4.03 (m, 1H), 3.98-3.90 (m, 6H), 3.89-3.68 (m, 4H), 3.32-3.15 (m, 4H), 2.97-2.84 (m, 5H), 2.81-2.66 (m, 4H), 2.52-2.42 (m, 2H), 2.16-2.14 (m, 1H), 2.12-2.05 (m, 3H), 1.97 (s, 2H), 1.74-1.60 (m, 4H), 1.29-1.17 (m, 12H).

LC-MS: MS (ESI⁺): t_{R}=3.572 min, m/z=966.7 [M+H⁺]

The compounds below were prepared in a similar manner as described in Example 39.

| | | | LC-MS |
|---|---|---|---|
| Compound | Name | ¹H NMR (CDCl₃) | [M + H⁺] |
| 287 | 2-[2-[5-acetyl-3-[7- | (DMSO-d₆) δ = 8.73 (s, 1H), 7.95-7.85 | 972.3 |
| | (difluoromethyl)-6-(1- | (m, 1H), 7.80-7.75 (m, 1H), 7.72-7.65 | |
| | methylpyrazol-4-yl)-3,4-dihydro- | (m, 1H), 7.49 (s, 1H), 7.30-7.20 (m, | |
| | 2H-quinolin-1-yl]-6,7-dihydro- | 1H), 7.16-7.08 (m, 1H), 7.05-6.95 (m, | |
| | 4H-pyrazolo[4,3-c]pyridin-1-yl]- | 1H), 6.94-6.60 (m, 2H), 4.70-4.55 (m, | |
| | 9-azadispiro[3.1.5⁶.1⁴]dodecan-9- | 1H), 4.28 (s, 1H), 4.20-4.08 (m, 2H), | |
| | yl]-N-[3-(3-chloro-4-cyano- | 4.05-3.98 (m, 1H), 3.90-3.85 (m, 3H), | |
| | phenoxy)-2,2,4,4-tetramethyl- | 3.82-3.73 (m, 4H), 3.70-3.55 (m, 4H), | |
| | cyclobutyl]pyrimidine-5- | 2.88-2.64 (m, 5H), 2.38-2.27 (m, 1H), | |
| | carboxamide | 2.15-2.02 (m, 5H), 1.98-1.86 (m, 6H), | |
| | | 1.55-1.47 (m, 4H), 1.20 (s, 6H), 1.10 | |
| | | (s, 6H). | |

### Example 40: Synthesis of 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-1-piperidyl]-N-[3-(4-cyano-3-methoxy-phenoxy)-2,2,4,4-tetramethyl-cyclobutyl]benzamide (Compound 258)

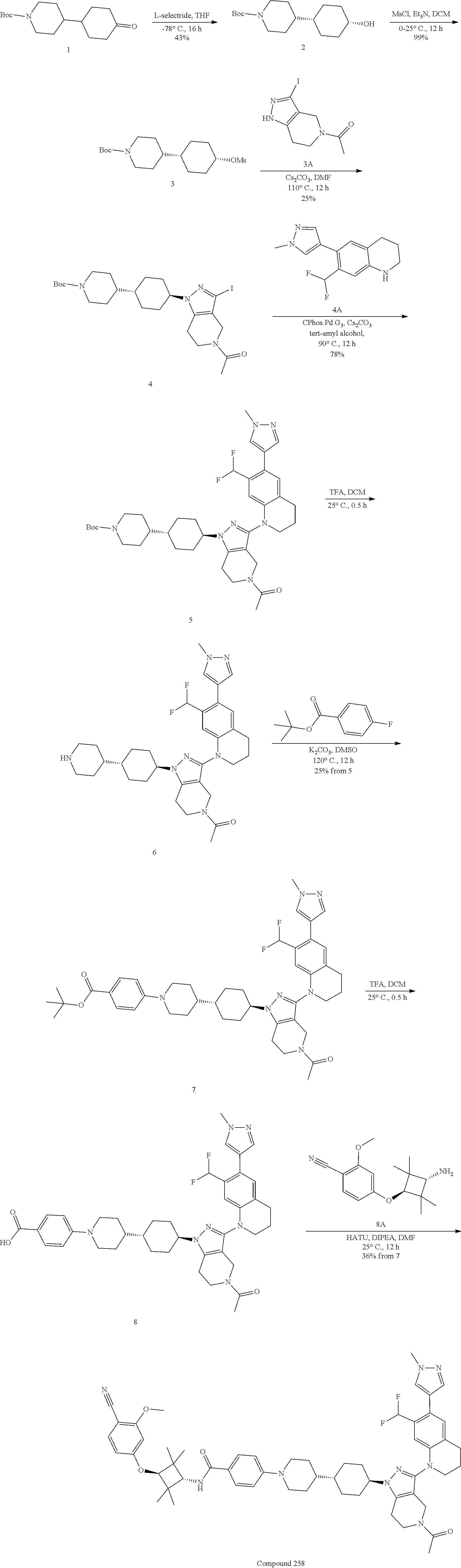

Synthesis of 1 was reported in ACS Medicinal Chemistry Letters, 2021, vol. 12, #7, p. 1108-1115.

To a solution of tert-butyl 4-(4-oxocyclohexyl)piperidine-1-carboxylate (6 g, 21.32 mmol, 1 eq) in THF (60 mL) was added L-selectride (1 M, 25.59 mL, 1.2 eq). The mixture was stirred at −78° C. for 16 h under N₂ atmosphere. The reaction mixture was diluted with ice water (100 mL). The mixture was diluted with water (50 mL) and extracted with ethyl acetate (100 mL×3). The combined organic layers were washed with brine (80 mL×3), dried over anhydrous sodium sulfate, filtered and concentrated in vacuum. The mixture was purified by prep-HPLC (column: Welch Ultimate XB—SiOH 250*50*10 um; mobile phase: [Hexane-EtOH]; B %: 5%, isocratic elution mode). Compound tert-butyl 4-(4-hydroxycyclohexyl)piperidine-1-carboxylate (2.8 g, 9.19 mmol, 43% yield) was obtained as a colorless oil.

¹H NMR (400 MHz, CDCl₃) δ=4.13 (d, J=7.0 Hz, 2H), 4.03-3.98 (m, 1H), 2.64 (t, J=12.1 Hz, 2H), 1.80-1.71 (m, 2H), 1.68 (d, J=12.8 Hz, 2H), 1.58-1.51 (m, 2H), 1.51-1.47 (m, 3H), 1.46 (s, 9H), 1.44 (s, 1H), 1.42-1.37 (m, 1H), 1.32-1.22 (m, 1H), 1.21-1.04 (m, 3H)

LC-MS: MS (ESI⁺): t_{R}=0.585 min, m/z=228.0 [M−55]

To a solution of tert-butyl 4-(4-hydroxycyclohexyl)piperidine-1-carboxylate (1.3 g, 4.59 mmol, 1 eq) tert-butyl 4-(4-hydroxycyclohexyl)piperidine-1-carboxylate (1.3 g, 4.59 mmol, 1 eq) in DCM (15 mL) was added Et₃N (2.32 g, 22.94 mmol, 3.19 mL, 5 eq) and MsCl (1.18 g, 10.30 mmol, 797.30 μL, 2.25 eq) at 0° C. The mixture was stirred at 25° C. for 12 h. The mixture was diluted with ice water (80 mL) and extracted with dichloromethane (100 mL×3). The combined organic layers were washed with brine (100 mL×3), dried over anhydrous sodium sulfate, filtered and concentrated in vacuum. The residue was purified by flash silica gel chromatography (ISCO®; 20 g SepaFlash® Silica Flash Column, Eluent of 0-16% petroleum ether/ethyl acetatelethergradient@80 mL/min). Compound tert-butyl 4-(4-methylsulfonyloxycyclohexyl)piperidine-1-carboxylate (1.65 g, 4.56 mmol, 99% yield) was obtained as a colourless oil.

¹H NMR (400 MHz, CDCl₃) δ=4.98 (s, 1H), 3.01 (s, 3H), 2.64 (t, J=11.9 Hz, 2H), 2.15-2.06 (m, 2H), 1.71-1.54 (m, 9H), 1.46 (s, 9H), 1.44-1.38 (m, 2H), 1.21-1.09 (m, 3H)

To a solution of tert-butyl 4-(4-methylsulfonyloxycyclohexyl)piperidine-1-carboxylate (1.61 g, 4.47 mmol, 1.3 eq) and 1-(3-iodo-1,4,6,7-tetrahydropyrazolo[4,3-c]pyridin-5-yl)ethanone (1 g, 3.44 mmol, 1 eq) in DMF (10 mL) was added Cs₂CO₃ (3.36 g, 10.31 mmol, 3 eq). The mixture was stirred at 110° C. for 12 h. The mixture was diluted with water (80 mL) and extracted with ethyl acetate (100 mL×3). The combined organic layers were washed with brine (100 mL×3), dried over anhydrous sodium sulfate, filtered and concentrated in vacuum. The mixture was purified by prep-HPLC (column: Welch Ultimate XB—CN 250*50*10 um; mobile phase: [Hexane-EtOH]; gradient: 1%-35% B over 15 min). Compound tert-butyl 4-[4-(5-acetyl-3-iodo-6,7-dihydro-4H-pyrazolo[4,3-c]pyridin-1-yl)cyclohexyl]piperidine-1-carboxylate (480 mg, 862.57 μmol, 25% yield) was obtained as a white solid.

LC-MS: MS (ESI⁺): t_{R}=0.583 min, m/z=557.1 [M+H⁺]

To a solution of tert-butyl 4-[4-(5-acetyl-3-iodo-6,7-dihydro-4H-pyrazolo[4,3-c]pyridin-1-yl)cyclohexyl]piperidine-1-carboxylate (429 mg, 771.32 μmol, 1 eq)and 7-(difluoromethyl)-6-(1-methylpyrazol-4-yl)-1,2,3,4-tetrahydroquinoline (243 mg, 925.58 μmol, 1.2 eq) in tert-amyl alcohol (10 mL) was added CPHOS PD G₃ (62 mg, 77.13 μmol, 0.1 eq) and Cs₂CO₃ (753 mg, 2.31 mmol, 3 eq). The reaction mixture was stirred at 90° C. for 12 h under N₂. The mixture was diluted with water (50 mL) and extracted with dichloromethane (60 mL×3). The combined organic layers were washed with brine (50 mL×3), dried over anhydrous sodium sulfate, filtered and concentrated in vacuum. The residue was purified by flash silica gel chromatography (ISCO®; 20 g SepaFlash® Silica Flash Column, Eluent of 0˜100% Ethylacetate/Petroleum ethergradient @100 mL/min).

Compound tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]piperidine-1-carboxylate (420 mg, 607.07 μmol, 78% yield) was obtained as a yellow solid.

LC-MS: MS (ESI⁺): t_{R}=0.620 min, m/z=692.4 [M+H⁺]

To a solution of tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]piperidine-1-carboxylate (350 mg, 505.89 μmol, 1 eq) in DCM (2 mL) was added TFA (3.07 g, 26.93 mmol, 2 mL, 53.22 eq). The mixture was stirred at 25° C. for 0.5 h. The mixture was concentrated in vacuo. Compound 1-[3-[7-(difluoromethyl)-6-(1-methylpyrazol-4-yl)-3,4-dihydro-2H-quinolin-1-yl]-1-[4-(4-piperidyl)cyclohexyl]-6,7-dihydro-4H-pyrazolo[4,3-c]pyridin-5-yl]ethanone (299 mg) was obtained as a yellow oil and directly used in the next step without further purification.

LC-MS: MS (ESI⁺): t_{R}=0.435 min, m/z=592.3 [M+H⁺]

To a solution of 1-[3-[7-(difluoromethyl)-6-(1-methylpyrazol-4-yl)-3,4-dihydro-2H-quinolin-1-yl]-1-[4-(4-piperidyl)cyclohexyl]-6,7-dihydro-4H-pyrazolo[4,3-c]pyridin-5-yl]ethanone (299 mg, 505.29 μmol, 1 eq) in DMSO (5 mL) was added K₂CO₃ (698 mg, 5.05 mmol, 10 eq), tert-butyl 4-fluorobenzoate (198 mg, 1.01 mmol, 2 eq). The mixture was stirred at 120° C. for 12 h. The mixture was diluted with water (20 mL) and extracted with ethyl acetate (30 mL×3). The combined organic layers were washed with brine (30 mL×3), dried over anhydrous sodium sulfate, filtered and concentrated in vacuum. The residue was purified by prep-TLC (SiO₂, DCM:MeOH=20:1). Compound tert-butyl 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-1-piperidyl]benzoate (100 mg, 130.22 μmol, 25% yield over two steps) was obtained as a yellow solid.

LC-MS: MS (ESI⁺): t_{R}=0.625 min, m/z=768.2 [M+H⁺]

To a solution of tert-butyl 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-1-piperidyl]benzoate (100 mg, 130.22 μmol, 1 eq) in DCM (2 mL) was added TFA (3.07 g, 26.93 mmol, 2 mL, 206.77 eq). The mixture was stirred at 25° C. for 0.5 h. The mixture was concentrated in vacuo. Compound 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-1-piperidyl]benzoic acid (92 mg) was obtained as a blue oil and directly used in the next step without further purification.

LC-MS: MS (ESI⁺): t_{R}=0.523 min, m/z=712.3 [M+H⁺]

To a solution of 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-1-piperidyl]benzoic acid (90 mg, 126.43 μmol, 1 eq) in DMF (3 mL) was added DIPEA (326 mg, 2.53 mmol, 440.44 μL, 20 eq), 4-(3-amino-2,2,4,4-tetramethyl-cyclobutoxy)-2-methoxy-benzonitrile (34 mg, 126.43 μmol, 1 eq) and HATU (144 mg, 379.30 μmol, 3 eq). The mixture was stirred at 25° C. for 12 h. The mixture was diluted with water (20 mL) and extracted with ethyl acetate (30 mL×3). The combined organic layers were washed with brine (20 mL×3), dried over anhydrous sodium sulfate, filtered and concentrated in vacuum. The residue was purified by prep-HPLC (column: Unisil 3-100 C18 Ultra 150*50 mm*3 um; mobile phase: [water(FA)-ACN]; gradient: 65%-95% B over 2 min). Compound 4-[4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-1-piperidyl]-N-[3-(4-cyano-3-methoxy-phenoxy)-2,2,4,4-tetramethyl-cyclobutyl]benzamide (44.47 mg, 45.93 μmol, 36% yield over two steps) was obtained as a white solid.

1H NMR (400 MHz, CDCl₃) δ=7.69 (d, J 8.8 Hz, 2H), 7.53 (d, J 5.3 Hz, 1H), 7.46 (d, J 8.6 Hz, 1H), 7.42-7.38 (m, 1H), 7.05-6.86 (2, 4H), 6.68-6.38 (m, 3H), 6.12 (d, J=8.1 Hz, 1H), 4.26 (s, 1H), 4.17-4.12 (m, 2H), 4.05 (s, 1H), 3.9 (d, J=2.3 Hz, 3H), 3.94-3.86 (m, 7H), 3.77-3.67 (m, 3H), 2.91-2.72 (m, 6H), 2.17 (s, 1H), 2.12-2.04 (m, 4H), 2.03-4 (m, 6H), 1.82 (br d, J=12. Hz, 2H), 1.42 (br s, 4H), 1.25 (d, J=10.1 Hz, 12H), 1.22-1.12 (4, 2H)

LC-MS: MS (ESI⁺): t_{R}=3.557 min, m/z=968.7 [M+H⁺],

The compounds below were prepared in a similar manner as described in Example 40.

| | | | LC-MS |
|---|---|---|---|
| Compound | Name | ¹H NMR (CDCl₃) | [M + H⁺] |
| 259 | 4-[4-[4-[5-acetyl-3-[7- | δ = 7.68 (d, J = 8.9 Hz, 2H), 7.53 (d, J = | 968.7 |
| | (difluoromethyl)-6-(1- | 5.0 Hz, 1H), 7.46 (d, J = 8.5 Hz, 1H), | |
| | methylpyrazol-4-yl)-3,4- | 7.40 (d, J = 6.6 Hz, 1H), 7.06-6.90 (m, | |
| | dihydro-2H-quinolin-1-yl]-6,7- | 1H), 6.68-6.38 (m, 3H), 6.11 (d, J = 8.1 | |
| | dihydro-4H-pyrazolo[4,3- | Hz, 1H), 4.30 (s, 1H), 4.17-4.13 (m, | |
| | c]pyridin-1-yl]cyclohexyl]-1- | 2H), 4.07-3.99 (m, 2H), 3.95 (d, J = 2.6 | |
| | piperidyl]-N-[3-(4-cyano-3- | Hz, 3H), 3.94-3.90 (m, 4H), 3.90-3.84 | |
| | methoxy-phenoxy)-2,2,4,4- | (m, 2H), 3.77-3.68 (m, 3H), 2.92-2.77 | |
| | tetramethyl- | (m, 5H), 2.74 (br t, J = 5.7 Hz, 1H), 2.18 | |
| | cyclobutyl]benzamide | (s, 1H), 2.15-2.04 (m, 6H), 2.02-1.90 | |
| | | (m, 4H), 1.81-1.69 (m, 3H), 1.53 (br s, | |
| | | 4H), 1.29 (br s, 3H), 1.25 (d, J = 9.9 Hz, | |
| | | 13H) | |
| 304 | 4-[4-[4-[5-acetyl-3-[7- | (DMSO-d₆): δ = 7.81-7.68 (m, 3H), | 983.7 |
| | (difluoromethyl)-6-(1- | 7.65 (d, J = 8.8 Hz, 1H), 7.54-7.46 (m, | |
| | methylpyrazol-4-yl)-3,4- | 2H), 7.12 (s, 1H), 6.87 (d, J = 44.0 Hz, | |
| | dihydro-2H-quinolin-1-yl]-6,7- | 1H), 6.53 (m, J = 2.0, 8.8 Hz, 1H), 4.27 | |
| | dihydro-4H-pyrazolo[4,3- | (s, 1H), 4.20-4.10 (m, 2H), 4.05 (d, J = | |
| | c]pyridin-1-yl]-1-piperidyl]-3- | 9.2 Hz, 1H), 3.93-3.81 (m, 8H), 3.80- | |
| | methyl-1-piperidyl]-N-[3-(4- | 3.65 (m, 3H), 3.62-3.55 (m, 2H), 3.35 | |
| | cyano-3-methoxy-phenoxy)- | (s, 3H), 3.20-3.02 (m, 2H), 2.97-2.68 | |
| | 2,2,4,4-tetramethyl- | (m, 7H), 2.26-1.81 (m, 11H), 1.25- | |
| | cyclobutyl]benzamide | 1.21 (m, 6H), 1.18-1.12 (m, 6H), 1.10- | |
| | | 0.86 (m, 3H). | |

### Example 41: Synthesis of 4-[4-[5-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-4,5,6,7-tetrahydroindazol-1-yl]-1-piperidyl]-N-[3-(4-cyano-3-methoxy-phenoxy)-2,2,4,4-tetramethyl-cyclobutyl]benzamide (Compound 265)

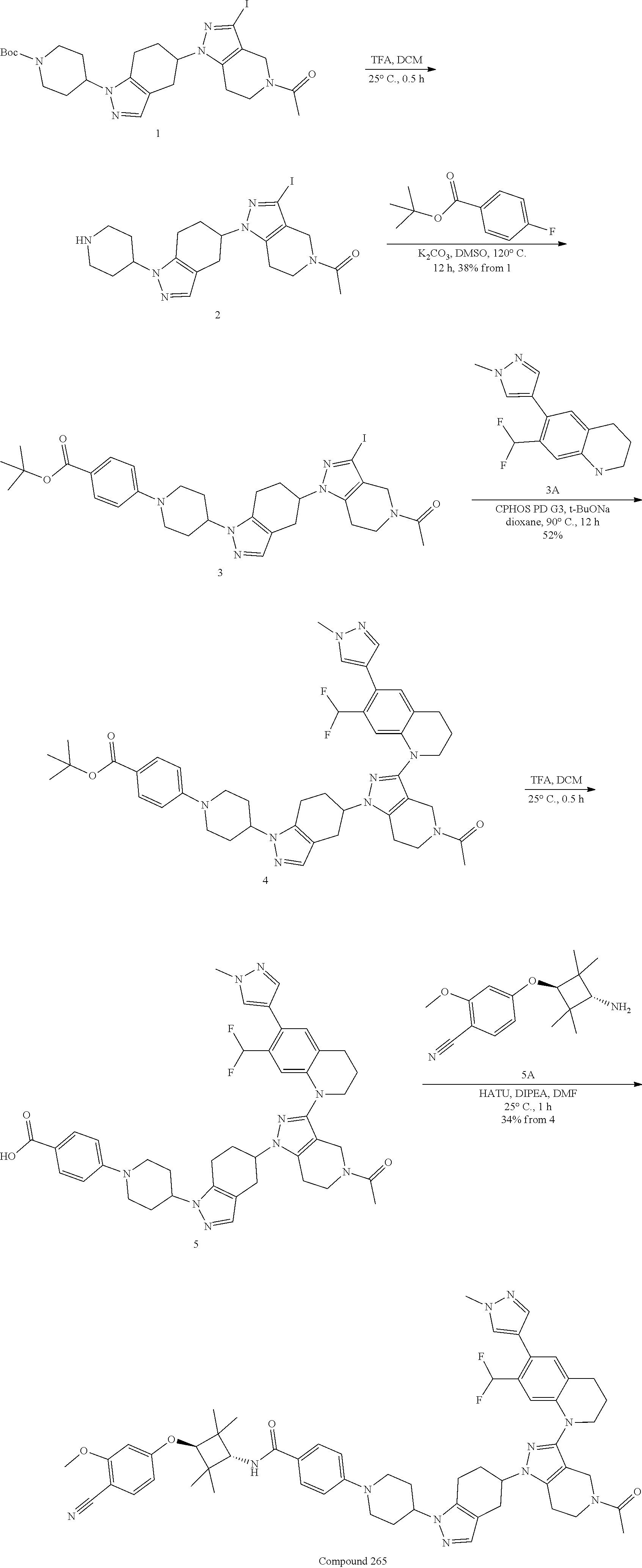

To a solution of tert-butyl 4-[5-(5-acetyl-3-iodo-6,7-dihydro-4H-pyrazolo[4,3-c]pyridin-1-yl)-4,5,6,7-tetrahydroindazol-1-yl]piperidine-1-carboxylate (270 mg, 454.17 μmol, 1 eq) in DCM (5 mL) was added TFA (259 mg, 2.27 mmol, 168.68 μL, 5 eq). The reaction mixture was stirred at 25° C. for 0.5 h. The reaction mixture was concentrated under reduced pressure to give a residue. Compound 1-[3-iodo-1-[1-(4-piperidyl)-4,5,6,7-tetrahydroindazol-5-yl]-6,7-dihydro-4H-pyrazolo[4,3-c]pyridin-5-yl]ethanone (230 mg, crude) was obtained as yellow oil and directly used in the next step without further purification.

LC-MS: MS (ESI⁺): t_{R}=0.396 min, m/z=495.0 [M+H⁺]

To a solution of 1-[3-iodo-1-[1-(4-piperidyl)-4,5,6,7-tetrahydroindazol-5-yl]-6,7-dihydro-4H-pyrazolo[4,3-c]pyridin-5-yl]ethanone (230 mg, 465.24 μmol, 1 eq) and K₂CO₃ (643 mg, 4.65 mmol, 10 eq) in DMSO (5 mL) was added tert-butyl 4-fluorobenzoate (456 mg, 2.33 mmol, 5 eq). The reaction mixture was stirred at 120° C. for 12 h. The reaction mixture was quenched by addition water (10 mL) at 0° C., and then diluted with water (30 mL) and extracted with ethyl acetate (50 mL×3). The combined organic layers were washed with brine (50 mL×3), dried over anhydrous sodium sulfate, filtered and concentrated under reduced pressure to give a residue. The residue was purified by prep-TLC (silica, DCM:MeOH=20:1). Compound tert-butyl 4-[4-[5-(5-acetyl-3-iodo-6,7-dihydro-4H-pyrazolo[4,3-c]pyridin-1-yl)-4,5,6,7-tetrahydroindazol-1-yl]-1-piperidyl]benzoate (120 mg, 178.95 μmol, 38% yield over two steps) was obtained as light yellow solid.

LC-MS: MS (ESI⁺): t_{R}=0.578 min, m/z=671.1 [M+H⁺]

To a solution of tert-butyl 4-[4-[5-(5-acetyl-3-iodo-6,7-dihydro-4H-pyrazolo[4,3-c]pyridin-1-yl)-4,5,6,7-tetrahydroindazol-1-yl]-1-piperidyl]benzoate (110 mg, 164.04 μmol, 1 eq) and 7-(difluoromethyl)-6-(1-methylpyrazol-4-yl)-1,2,3,4-tetrahydroquinoline (65 mg, 246.05 μmol, 1.5 eq) in dioxane (10 mL) was added CPHOS PD G₃ (13 mg, 16.40 μmol, 0.1 eq) and t-BuONa (2 M, 246.05 μL, 3 eq). The reaction mixture was stirred at 90° C. for 12 h under N₂. The reaction mixture was concentrated under reduced pressure to give a residue. The residue was purified by prep-TLC (silica, DCM:MeOH=20:1). Compound tert-butyl 4-[4-[5-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-4,5,6,7-tetrahydroindazol-1-yl]-1-piperidyl]benzoate (70 mg, 86.85 μmol, 52% yield) was obtained as yellow oil.

LC-MS: MS (ESI⁺): t_{R}=0.597 min, m/z=806.3 [M+H⁺]

To a solution of tert-butyl 4-[4-[5-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-4,5,6,7-tetrahydroindazol-1-yl]-1-piperidyl]benzoate (70 mg, 86.85 μmol, 1 eq) in DCM (3 mL) was added TFA (50 mg, 434.27 μmol, 32.26 μL, 5 eq). The reaction mixture was stirred at 25° C. for 0.5 h. The reaction mixture was concentrated under reduced pressure to give a residue. Compound 4-[4-[5-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-4,5,6,7-tetrahydroindazol-1-yl]-1-piperidyl]benzoic acid (65 mg) was obtained as yellow oil and directly used in the next step without further purification.

LC-MS: MS (ESI⁺): t_{R}=0.509 min, m/z=750.4 [M+H⁺]

To a solution of 4-[4-[5-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-4,5,6,7-tetrahydroindazol-1-yl]-1-piperidyl]benzoic acid (65 mg) and DIPEA (111 mg, 861.17 μmol, 0.15 mL, 9.93 eq) in DMF (3 mL) was added 4-(3-amino-2,2,4,4-tetramethyl-cyclobutoxy)-2-methoxy-benzonitrile (24 mg, 86.68 μmol, 1 eq) and HATU (66 mg, 173.37 μmol, 2 eq). The reaction mixture was stirred at 25° C. for 1 h. The reaction mixture was quenched by addition water (10 mL) at 25° C., and then diluted with water (20 mL) and extracted with ethyl acetate (30 mL×3). The combined organic layers were washed with brine (30 mL×3), dried over anhydrous sodium sulfate, filtered and concentrated under reduced pressure to give a residue. The residue was purified by prep-TLC (silica, DCM:MeOH=20:1). The residue was purified by prep-HPLC (FA condition: column: Unisil 3-100 C18 Ultra 150*50 mm*3 um; mobile phase: [water(FA)-ACN]; gradient: 55%-85% B over 10 min). Compound 4-[4-[5-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-4,5,6,7-tetrahydroindazol-1-yl]-1-piperidyl]-N-[3-(4-cyano-3-methoxy-phenoxy)-2,2,4,4-tetramethyl-cyclobutyl]benzamide (30.66 mg, 30.23 μmol, 34% yield over two steps) was obtained as a white solid.

¹H NMR (400 MHz, CDCl₃) δ=7.70 (d, J=8.8 Hz, 2H), 7.54 (d, J=5.6 Hz, 1H), 7.46 (d, J=8.8 Hz, 1H), 7.41 (d, J=6.8 Hz, 1H), 7.33-7.29 (m, 1H), 7.08-6.99 (m, 1H), 6.96 (d, J=8.8 Hz, 2H), 6.92-6.87 (m, 1H), 6.70-6.45 (m, 2H), 6.41 (dd, J=2.0, 8.8 Hz, 1H), 6.13 (d, J=8.0 Hz, 1H), 4.37-4.21 (m, 2H), 4.19-4.12 (m, 3H), 4.07-3.99 (m, 3H), 3.96 (d, J=2.4 Hz, 3H), 3.94-3.83 (m, 4H), 3.81-3.66 (m, 3H), 3.17-3.08 (m, 1H), 3.02 (t, J=12.8 Hz, 2H), 2.96-2.73 (m, 7H), 2.58-2.46 (m, 1H), 2.43-2.26 (m, 3H), 2.18 (s, 1H), 2.11-1.98 (m, 6H), 1.26 (d, J=10.8 Hz, 12H)

LC-MS: MS (ESI⁺): t_{R}=3.476 min, m/z=1006.7 [M+H⁺]

### Example 42: Synthesis of 4-[3-[5-[8-(5-acetyl-1-tetrahydropyran-4-yl-6,7-dihydro-4H-pyrazolo[4,3-c]pyridin-3-yl)-3-isoquinolyl]-2-pyridyl]-3,9-diazaspiro[5.5]undecan-9-yl]-N-[3-(4-cyano-3-methoxy-phenoxy)-2,2,4,4-tetramethyl-cyclobutyl]benzamide (Compound 266)

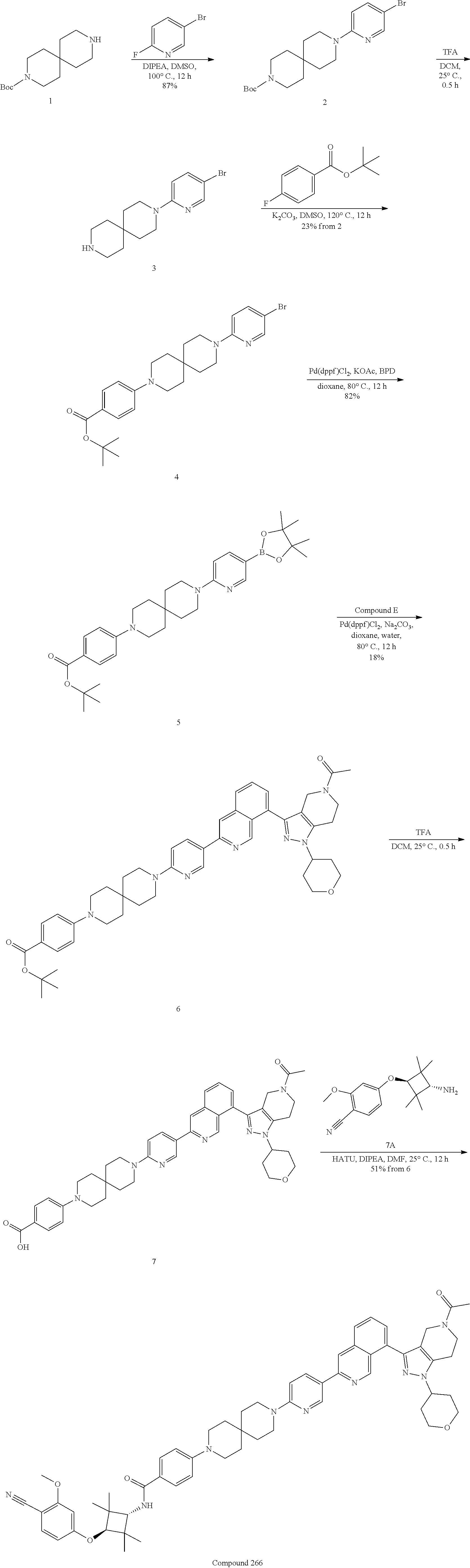

To a solution of tert-butyl 3,9-diazaspiro[5.5]undecane-3-carboxylate (3.00 g, 11.79 mmol, 1 eq) in DMSO (30 mL) was added DIPEA (4.57 g, 35.38 mmol, 6.16 mL, 3 eq) and 5-bromo-2-fluoro-pyridine (2.08 g, 11.79 mmol, 1.21 mL, 1 eq). The mixture was stirred at 100° C. for 12 h. The mixture was poured into water (100 mL) and extracted with ethyl acetate (100 mL×3). The combined organic phase was washed with brine (100 mL×3), dried with anhydrous sodium sulfate, filtered and concentrated in vacuum. The residue was purified by column chromatography (SiO₂, petroleum ether/ethyl acetate=50/1 to 10/1). Compound tert-butyl 9-(5-bromo-2-pyridyl)-3,9-diazaspiro[5.5]undecane-3-carboxylate (4.20 g, 10.24 mmol, 87% yield) was obtained as a yellow solid.

To a solution of tert-butyl 9-(5-bromo-2-pyridyl)-3,9-diazaspiro[5.5]undecane-3-carboxylate (4.50 g, 10.97 mmol, 1 eq) in DCM (40 mL) was added TFA (58.79 g, 515.57 mmol, 38.30 mL, 47.01 eq). The mixture was stirred at 25° C. for 0.5 h. The mixture was concentrated in vacuum. Compound 3-(5-bromo-2-pyridyl)-3,9-diazaspiro[5.5]undecane (4.50 g) was obtained as a yellow oil and directly used in the next step without further purification.

To a solution of 3-(5-bromo-2-pyridyl)-3,9-diazaspiro[5.5]undecane (4.50 g) in DMSO (60 mL) was added K₂CO₃ (14.60 g, 106 mmol, 10 eq) and tert-butyl 4-fluorobenzoate (2.08 g, 10.61 mmol, 1 eq). The mixture was stirred at 120° C. for 12 h. The mixture was poured into water (100 mL) and extracted with dichloromethane (100 mL×3). The combined organic phase was washed with brine (100 mL×6), dried with anhydrous sodium sulfate, filtered and concentrated in vacuum. The residue was purified by column chromatography (SiO₂, petroleum ether/ethyl acetate=50/1 to 3/1). Compound tert-butyl 4-[3-(5-bromo-2-pyridyl)-3,9-diazaspiro[5.5]undecan-9-yl]benzoate (1.20 g, 2.47 mmol, 23% yield) was obtained as a yellow solid.

LC-MS: MS (ESI+): t_{R}=0.587 min, m/z=488.2 [M+H⁺]

To a solution of tert-butyl 4-[3-(5-bromo-2-pyridyl)-3,9-diazaspiro[5.5]undecan-9-yl]benzoate (900 mg, 1.85 mmol, 1 eq) and BPD (563 mg, 2.22 mmol, 1.2 eq) in dioxane (15 mL) was added KOAc (363 mg, 3.70 mmol, 2 eq) and Pd(dppf)Cl₂ (67 mg, 92 μmol, 0.05 eq). The mixture was stirred at 80° C. for 12 h under nitrogen atmosphere. The mixture was filtered, washed with dichloromethane (100 mL) and concentrated in vacuum. The residue was purified by column chromatography (SiO₂, petroleum ether/ethyl acetate=50/1 to 0/1). Compound tert-butyl 4-[3-[5-(4,4,5,5-tetramethyl-1,3,2-dioxaborolan-2-yl)-2-pyridyl]-3,9-diazaspiro[5.5]undecan-9-yl]benzoate (800 mg, 1.50 mmol, 81% yield) was obtained as a yellow solid.

To a solution of tert-butyl 4-[3-[5-(4,4,5,5-tetramethyl-1,3,2-dioxaborolan-2-yl)-2-pyridyl]-3,9-diazaspiro[5.5]undecan-9-yl]benzoate (300 mg, 562 μmol, 1 eq) and [8-(5-acetyl-1-tetrahydropyran-4-yl-6,7-dihydro-4H-pyrazolo[4,3-c]pyridin-3-yl)-3-isoquinolyl]trifluoromethanesulfonate (97 mg, 185 μmol, 0.33 eq) in dioxane (5 mL) and water (0.5 mL) was added Na₂CO₃ (119 mg, 1.12 mmol, 2 eq) and Pd(dppf)Cl₂ (20 mg, 28 μmol, 0.05 eq). The mixture was stirred at 80° C. for 12 h under nitrogen atmosphere. The mixture was poured into water (50 mL) and extracted with dichloromethane (50 mL×3). The combined organic phase was washed with brine (100 mL×2), dried with anhydrous sodium sulfate, filtered and concentrated in vacuum. The residue was purified by prep-TLC (SiO₂, dichloromethane/methanol=15/1). Compound tert-butyl 4-[3-[5-[8-(5-acetyl-1-tetrahydropyran-4-yl-6,7-dihydro-4H-pyrazolo[4,3-c]pyridin-3-yl)-3-isoquinolyl]-2-pyridyl]-3,9-diazaspiro[5.5]undecan-9-yl]benzoate (80 mg, 102 μmol, 18% yield) was obtained as a yellow solid.

LC-MS: MS (ESI+): t_{R}=0.520 min, m/z=782.4 [M+H⁺]

To a solution of tert-butyl 4-[3-[5-[8-(5-acetyl-1-tetrahydropyran-4-yl-6,7-dihydro-4H-pyrazolo[4,3-c]pyridin-3-yl)-3-isoquinolyl]-2-pyridyl]-3,9-diazaspiro[5.5]undecan-9-yl]benzoate (80 mg, 102 μmol, 1 eq) in DCM (1 mL) was added TFA (1.54 g, 13.46 mmol, 1 mL, 131.59 eq). The mixture was stirred at 25° C. for 0.5 h. The mixture was concentrated in vacuum. Compound 4-[3-[5-[8-(5-acetyl-1-tetrahydropyran-4-yl-6,7-dihydro-4H-pyrazolo[4,3-c]pyridin-3-yl)-3-isoquinolyl]-2-pyridyl]-3,9-diazaspiro[5.5]undecan-9-yl]benzoic acid (75 mg) was obtained as a yellow oil and directly used in the next step without further purification.

To a solution of 4-[3-[5-[8-(5-acetyl-1-tetrahydropyran-4-yl-6,7-dihydro-4H-pyrazolo[4,3-c]pyridin-3-yl)-3-isoquinolyl]-2-pyridyl]-3,9-diazaspiro[5.5]undecan-9-yl]benzoic acid (75 mg) and DIPEA (34 mg, 267 μmol, 46 μL, 3 eq) in DMF (1 mL) was added 4-(3-amino-2,2,4,4-tetramethyl-cyclobutoxy)-2-methoxy-benzonitrile (24 mg, 89 μmol, 1 eq) and HATU (67 mg, 178 μmol, 2 eq). The mixture was stirred at 25° C. for 12 h. The mixture was poured into water (50 mL) and extracted with dichloromethane (50 mL×3). The combined organic phase was washed with brine (100 mL×3), dried with anhydrous sodium sulfate, filtered and concentrated in vacuum. The residue was purified by prep-TLC (SiO₂, dichloromethane/methanol=15/1) and by prep-HPLC (column: Phenomenex luna C18 150*25 mm*10 um; mobile phase: [water(FA)-ACN]; gradient: 35%-65% B over 9 min). Compound 4-[3-[5-[8-(5-acetyl-1-tetrahydropyran-4-yl-6,7-dihydro-4H-pyrazolo[4,3-c]pyridin-3-yl)-3-isoquinolyl]-2-pyridyl]-3,9-diazaspiro[5.5]undecan-9-yl]-N-[3-(4-cyano-3-methoxy-phenoxy)-2,2,4,4-tetramethyl-cyclobutyl]benzamide (47.5 mg, 45 μmol, 51% yield over two steps) was obtained as a yellow solid.

¹H NMR (400 MHz, CHLOROFORM-d) δ=9.76 (d, J=15.2 Hz, 1H) 8.93 (dd, J=6.0, 2.0 Hz, 1H) 8.33 (s, 1H) 8.00 (d, J=9.2 Hz, 1H) 7.91-7.81 (m, 1H) 7.76-7.72 (m, 1H) 7.72-7.67 (m, 2H) 7.57-7.48 (m, 1H) 7.46 (d, J=8.4 Hz, 1H) 6.94 (d, J=8.8 Hz, 2H) 6.83 (d, J=9.2 Hz, 1H) 6.47 (d, J=2.0 Hz, 1H) 6.41 (dd, J=8.8, 2.0 Hz, 1H) 6.13 (d, J=8.0 Hz, 1H) 4.66 (s, 1H) 4.49 (s, 1H) 4.34-4.24 (m, 1H) 4.21-4.12 (m, 3H) 4.07-3.99 (m, 2H) 3.85 (t, J=5.6 Hz, 1H) 3.70 (s, 4H) 3.63-3.52 (m, 2H) 3.43-3.30 (m, 4H) 2.98-2.90 (m, 1H) 2.87 (t, J=5.6 Hz, 1H) 2.53-2.38 (m, 2H) 2.22 (s, 1H) 2.08 (s, 2H) 1.98 (d, J=13.2 Hz, 4H) 1.72 (dt, J=14.8, 5.6 Hz, 8H) 1.27 (s, 6H) 1.24 (s, 6H).

LC-MS: MS (ESI+): t_{R}=2.744 min, m/z=982.6 [M+H⁺]

The compounds below were prepared in a similar manner as described in Example 42.

| | | | LC-MS |
|---|---|---|---|
| Compound | Name | ¹H NMR (CDCl₃) | [M + H⁺] |
| 267 | 4-[4-[1-[5-[8-(5-acetyl-1- | δ = 9.73 (d, J = 14.0 Hz, 1H), 8.90 | 996.6 |
| | tetrahydropyran-4-yl-6,7-dihydro-4H- | (dd, J = 2.4, 6.0 Hz, 1H), 8.27 (ddd, | |
| | pyrazolo[4,3-c]pyridin-3-yl)-3- | J = 2.4, 6.4, 8.8 Hz, 1H), 7.96 (d, J = | |
| | isoquinolyl]-2-pyridyl]-4-piperidyl]- | 8.8 Hz, 1H), 7.89-7.79 (m, 1H), 7.75- | |
| | 1-piperidyl]-N-[3-(4-cyano-3- | 7.64 (m, 3H), 7.57-7.42 (m, 2H), | |
| | methoxy-phenoxy)-2,2,4,4- | 6.92 (d, J = 9.2 Hz, 2H), 6.81 (br d, | |
| | tetramethyl-cyclobutyl]benzamide | J = 8.8 Hz, 1H), 6.47 (d, J = 2.0 Hz, | |
| | | 1H), 6.40 (dd, J = 2.0, 8.4 Hz, 1H), | |
| | | 6.12 (d, J = 8.0 Hz, 1H), 4.65 (s, 1H), | |
| | | 4.52-4.42 (m, 3H), 4.28 (dt, J = 3.6, | |
| | | 12.0 Hz, 1H), 4.21-4.11 (m, 3H), | |
| | | 4.06-3.99 (m, 2H), 3.96-3.81 (m, | |
| | | 6H), 3.57 (br t, J = 12.0 Hz, 2H), 2.96- | |
| | | 2.73 (m, 6H), 2.52-2.39 (m, 2H), | |
| | | 2.22-2.06 (m, 3H), 1.97 (br d, J = | |
| | | 12.4 Hz, 2H), 1.86 (br d, J = 11.6 Hz, | |
| | | 4H), 1.45-1.33 (m, 6H), 1.25 (d, J = | |
| | | 10.4 Hz, 12H) | |
| 268 | 4-[4-[1-[5-[8-(5-acetyl-1- | δ = 9.74 (d, J = 13.2 Hz, 1H), 8.94- | 968.7 |
| | tetrahydropyran-4-yl-6,7-dihydro-4H- | 8.80 (m, 1H), 8.27 (dd, J = 6.4, 8.6 | |
| | pyrazolo[4,3-c]pyridin-3-yl)-3- | Hz, 1H), 7.95 (d, J = 9.6 Hz, 1H), | |
| | isoquinolyl]-2-pyridyl]azetidin-3-yl]- | 7.90-7.79 (m, 1H), 7.76-7.67 (m, | |
| | 1-piperidyl]-N-[3-(4-cyano-3- | 3H), 7.54-7.44 (m, 2H), 6.93 (d, J = | |
| | methoxy-phenoxy)-2,2,4,4- | 8.8 Hz, 2H), 6.47 (d, J = 2.0 Hz, 1H), | |
| | tetramethyl-cyclobutyl]benzamide | 6.45-6.37 (m, 2H), 6.12 (d, J = 8.0 | |
| | | Hz, 1H), 4.65 (s, 1H), 4.48 (s, 1H), | |
| | | 4.34-4.25 (m, 1H), 4.24-4.14 (m, | |
| | | 5H), 4.05 (s, 1H), 4.02 (br t, J = 6.0 | |
| | | Hz, 1H), 3.92 (s, 3H), 3.90-3.83 (m, | |
| | | 4H), 3.57 (br t, J = 12.0 Hz, 2H), 2.95- | |
| | | 2.82 (m, 4H), 2.63-2.54 (m, 1H), | |
| | | 2.51-2.40 (m, 2H), 2.21 (s, 1H), 2.08 | |
| | | (s, 2H), 1.97 (d, J = 12.4 Hz, 2H), | |
| | | 1.85 (d, J = 13.2 Hz, 3H), 1.44-1.30 | |
| | | (m, 3H), 1.25 (d, J = 10.4 Hz, 12H) | |
| 269 | 4-[9-[4-[8-(5-acetyl-1- | δ = 9.71 (d, J = 12.4 Hz, 1H), 8.15 (d, | 970.6 |
| | tetrahydropyran-4-yl-6,7-dihydro-4H- | J = 4.4 Hz, 1H), 8.08 (d, J = 2.0 Hz, | |
| | pyrazolo[4,3-c]pyridin-3-yl)-3- | 1H), 7.87-7.76 (m, 2H), 7.74-7.65 | |
| | isoquinolyl]pyrazol-1-yl]-3- | (m, 3H), 7.54-7.43 (m, 2H), 6.93 (d, | |
| | azaspiro[5.5]undecan-3-yl]-N-[3-(4- | J = 8.8 Hz, 2H), 6.47 (d, J = 2.0 Hz, | |
| | cyano-3-methoxy-phenoxy)-2,2,4,4- | 1H), 6.40 (dd, J = 2.4, 7.6 Hz, 1H), | |
| | tetramethyl-cyclobutyl]benzamide | 6.13 (d, J = 8.4 Hz, 1H), 4.66 (s, 1H), | |
| | | 4.49 (s, 1H), 4.33-4.12 (m, 5H), 4.07- | |
| | | 3.99 (m, 2H), 3.92 (s, 3H), 3.85 (t, | |
| | | J = 5.6 Hz, 1H), 3.57 (t, J = 12.0 Hz, | |
| | | 2H), 3.33 (q, J = 5.2 Hz, 4H), 2.97- | |
| | | 2.82 (m, 2H), 2.52-2.37 (m, 2H), | |
| | | 2.22-2.04 (m, 7H), 1.95 (t, J = 12.8 | |
| | | Hz, 4H), 1.84 (d, J = 5.6 Hz, 2H), | |
| | | 1.64-1.57 (m, 2H), 1.44-1.36 (m, | |
| | | 2H), 1.25 (d, J = 11.2 Hz, 12H) | |
| 270 | 4-[4-[4-[4-[8-(5-acetyl-1- | δ = 9.68 (s, 1H), 8.23-8.03 (m, 2H), | 985.6 |
| | tetrahydropyran-4-yl-6,7-dihydro-4H- | 7.90-7.65 (m, 5H), 7.57-7.42 (m, | |
| | pyrazolo[4,3-c]pyridin-3-yl)-3- | 2H), 7.02-6.80 (m, 2H), 6.56-6.37 | |
| | isoquinolyl]pyrazol-1-yl]-1- | (m, 2H), 6.17 (s, 1H), 4.78-4.49 (m, | |
| | piperidyl]-1-piperidyl]-N-[3-(4- | 2H), 4.49-4.21 (m, 3H), 4.21-4.10 | |
| | cyano-3-methoxy-phenoxy)-2,2,4,4- | (m, 3H), 4.09-3.96 (m, 3H), 3.92 (s, | |
| | tetramethylcyclobutyl]benzamide | 4H), 3.89-3.81 (m, 1H), 3.63-3.51 | |
| | | (m, 2H), 3.50-3.29 (m, 2H), 2.99- | |
| | | 2.82 (m, 5H), 2.57-2.37 (m, 5H), | |
| | | 2.35-2.23 (m, 3H), 2.21 (s, 2H), | |
| | | 2.00-1.91 (m, 3H), 1.89-1.72 (m, | |
| | | 3H), 1.34-1.23 (m, 12H) | |
| 271 | 4-[4-[4-[4-[8-(5-acetyl-1- | δ = 9.68 (d, J = 11.2 Hz, 1H), 8.17- | 984.8 |
| | tetrahydropyran-4-yl-6,7-dihydro-4H- | 7.99 (m, 2H), 7.88-7.76 (m, 2H), | |
| | pyrazolo[4,3-c]pyridin-3-yl)-3- | 7.73-7.63 (m, 3H), 7.51-7.42 (m, | |
| | isoquinolyl]pyrazol-1-yl]cyclohexyl]- | 2H), 6.93 (d, J = 8.8Hz, 2H), 6.49- | |
| | 1-piperidyl]-N-[3-(4-cyano-3- | 6.38 (m, 2H), 6.13 (d, J = 8.0 Hz, 1H), | |
| | methoxy-phenoxy)-2,2,4,4- | 4.79 (s, 8H), 4.66-4.47 (m, 2H), 4.32- | |
| | tetramethylcyclobutyl]benzamide | 4.24 (m, 1H), 4.20-4.11 (m, 4H), | |
| | | 4.05 (s, 1H), 4.01 (t, J = 5.6 Hz, 1H), | |
| | | 3.95-3.90 (m, 4H), 3.89-3.81 (m, | |
| | | 2H), 3.57 (t, J = 12.0 Hz, 2H), 2.93 (t, | |
| | | J = 5.6 Hz, 1H), 2.86 (t, J = 5.6 Hz, | |
| | | 1H), 2.84-2.75 (m, 2H), 2.51-2.38 | |
| | | (m, 2H), 2.33 (d, J = 10.4 Hz, 2H), | |
| | | 2.21 (s, 1H), 2.07 (s, 2H), 1.98 (s, | |
| | | 2H), 1.86-1.83 (m, 2H), 1.45-1.39 | |
| | | (m, 2H), 1.25 (d, J = 10.4 Hz, 12H). | |
| 272 | 4-[9-[5-acetyl-3-[7-(difluoromethyl)- | δ = 7.69 (d, J = 8.8 Hz, 2H), 7.54 (d, | 954.6 |
| | 6-(1-methylpyrazol-4-yl)-3,4- | J = 5.2 Hz, 1H), 7.46 (d, J = 8.4 Hz, | |
| | dihydro-2H-quinolin-1-yl]-6,7- | 1H), 7.43-7.38 (m, 1H), 7.07-6.98 | |
| | dihydro-4H-pyrazolo[4,3-c]pyridin-1- | (m, 1H), 6.92 (d, J = 10.0 Hz, 3H), | |
| | yl]-3-azaspiro[5.5]undecan-3-yl]-N- | 6.69-6.37 (m, 3H), 6.12 (d, J = 8.4 | |
| | [3-(4-cyano-3-methoxy-phenoxy)- | Hz, 1H), 4.31-4.11 (m, 3H), 4.05 (s, | |
| | 2,2,4,4-tetramethyl- | 1H), 3.96 (d, J = 2.0 Hz, 3H), 3.94- | |
| | cyclobutyl]benzamide | 3.86 (m, 5H), 3.80-3.66 (m, 3H), | |
| | | 3.32 (t, J = 10.4 Hz, 4H), 2.92-2.85 | |
| | | (m, 2H), 2.84-2.71 (m, 2H), 2.24- | |
| | | 2.14 (m, 3H), 2.13-2.03 (m, 4H), | |
| | | 1.92 (d, J = 13.2 Hz, 2H), 1.87-1.78 | |
| | | (m, 4H), 1.58 (s, 2H), 1.33 (d, J = | |
| | | 2.8 Hz, 2H), 1.25 (d, J = 10.0 Hz, | |
| | | 12H). | |
| 321 | 4-[4-[1-[5-[8-(5-acetyl-1- | δ = 9.73 (d, J = 12.0 Hz, 1H), 8.91 | 997.6 |
| | tetrahydropyran-4-yl-6,7-dihydro-4H- | (dd, J = 2.4, 4.6 Hz, 1H), 8.33-8.22 | |
| | pyrazolo[4,3-c]pyridin-3-yl)-3- | (m, 1H), 7.97 (d, J = 9.6 Hz, 1H), | |
| | isoquinolyl]-2-pyridyl]-4- | 7.89-7.80 (m, 1H), 7.76-7.66 (m, | |
| | piperidyl]piperazin-1-yl]-N-[3-(4- | 3H), 7.55-7.42 (m, 2H), 6.93 (d, J = | |
| | cyano-3-methoxy-phenoxy)-2,2,4,4- | 8.8 Hz, 2H), 6.82 (dd, J = 2.4, 8.9 Hz, | |
| | tetramethyl-cyclobutyl]benzamide | 1H), 6.47 (d, J = 2.0 Hz, 1H), 6.40 | |
| | | (dd, J = 2.4, 8.6 Hz, 1H), 6.13 (d, J = | |
| | | 8.0 Hz, 1H), 4.65 (s, 1H), 4.53-4.45 | |
| | | (m, 3H), 4.34-4.24 (m, 1H), 4.20- | |
| | | 4.12 (m, 3H), 4.06-4.00 (m, 2H), | |
| | | 3.92 (s, 3H), 3.85 (t, J = 5.6 Hz, 1H), | |
| | | 3.57 (t, J = 12.0 Hz, 2H), 3.39-3.28 | |
| | | (m, 4H), 3.04-2.89 (m, 3H), 2.87 (t, | |
| | | J = 5.4 Hz, 1H), 2.82-2.73 (m, 4H), | |
| | | 2.67-2.58 (m, 1H), 2.51-2.39 (m, | |
| | | 2H), 2.21 (s, 1H), 2.11-1.93 (m, 6H), | |
| | | 1.68-1.62 (m, 2H), 1.25 (d, J = 9.6 | |
| | | Hz, 12H) | |
| 322 | 4-[4-[1-[5-[8-(5-acetyl-1- | δ = 9.74 (d, J = 12.8 Hz, 1H), 8.88 | 969.6 |
| | tetrahydropyran-4-yl-6,7-dihydro-4H- | (dd, J = 5.8, 2.3 Hz, 1H), 8.33-8.23 | |
| | pyrazolo[4,3-c]pyridin-3-yl)-3- | (m, 1H), 7.96 (d, J = 9.8 Hz, 1H), | |
| | isoquinolyl]-2-pyridyl]azetidin-3- | 7.90-7.79 (m, 1H), 7.75-7.68 (m, 3H), | |
| | yl]piperazin-1-yl]-N-[3-(4-cyano-3- | 7.56-7.44 (m, 2H), 6.94 (d, J = 8.9 Hz, | |
| | methoxy-phenoxy)-2,2,4,4- | 2H), 6.49-6.43 (m, 2H), 6.40 (dd, J = | |
| | tetramethyl-cyclobutyl]benzamide | 8.6, 2.1 Hz, 1H), 6.13 (d, J = 8.1 Hz, | |
| | | 1H), 4.65 (s, 1H), 4.48 (s, 1H), 4.36- | |
| | | 4.26 (m, 1H), 4.24 (t, J = 7.5 Hz, 2H), | |
| | | 4.20-4.14 (m, 3H), 4.07-4.00 (m, 4H), | |
| | | 3.92 (s, 3H), 3.85 (t, J = 5.6 Hz, 1H), | |
| | | 3.57 (t, J = 12.0 Hz, 2H), 3.48-3.42 | |
| | | (m, 1H), 3.40-3.33 (m, 4H), 2.94 (t, | |
| | | J = 5.8 Hz, 1H), 2.87 (t, J = 5.4 Hz, | |
| | | 1H), 2.68-2.58 (m, 4H), 2.51-2.40 (m, | |
| | | 2H), 2.21 (s, 1H), 2.08 (s, 2H), 1.97 ( | |
| | | d, J = 12.5 Hz, 2H), 1.25 (d, J = 9.9 | |
| | | Hz, 12H) | |

### Example 43: Synthesis of 4-[4-[1-[5-[l-(5-acetyl-1-tetrahydropyran-4-yl-6,7-dihydro-4H-pyrazolo[4,3-c]pyridin-3-yl)-3,4-dihydro-2H-1,7-naphthyridin-6-yl]-2-pyridyl]-4-piperidyl]piperazin-1-yl]-N-[3-(4-cyano-3-methoxy-phenoxy)-2,2,4,4-tetramethyl-cyclobutyl]benzamide (Compound 323)

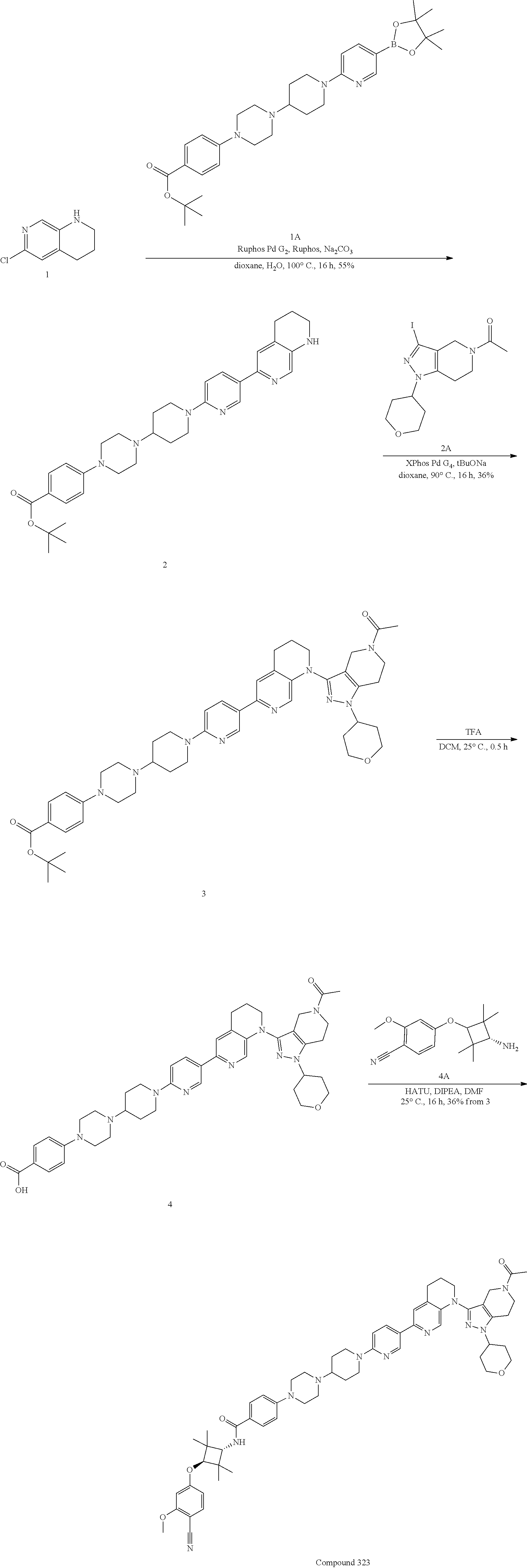

Synthesis of Compound 1 was reported in WO2022/20342 A1.

To a solution of 6-chloro-1,2,3,4-tetrahydro-1,7-naphthyridine (100 mg, 593.04 μmol, 1.0 eq) and tert-butyl 4-[4-[1-[5-(4,4,5,5-tetramethyl-1,3,2-dioxaborolan-2-yl)-2-pyridyl]-4-piperidyl]piperazin-1-yl]benzoate (400 mg, 729.23 μmol, 1.23 eq) in dioxane (10 mL) and H₂O (2 mL) was added sodium carbonate (189 mg, 1.78 mmol, 3.0 eq), RuPhos (28 mg, 59.30 μmol, 0.1 eq) and Ruphos Pd G₂ (46 mg, 59.30 μmol, 0.1 eq). The mixture was stirred at 100° C. for 16 h. The reaction mixture was quenched by addition water 50 mL, and then extracted with ethyl acetate (50 mL×3). The combined organic layers were washed with brine (50 mL×3), dried over anhydrous sodium sulfate, filtered and concentrated under reduced pressure to give a residue. The residue was purified by prep-TLC (SiO₂, dichloromethane:methanol=25:1). Compound tert-butyl 4-[4-[1-[5-(1,2,3,4-tetrahydro-1,7-naphthyridin-6-yl)-2-pyridyl]-4-piperidyl]piperazin-1-yl]benzoate (180 mg, 324.49 μmol, 55% yield) was obtained as a yellow solid.

LC-MS: MS (ESI⁺): t_{R}=0.465 min, m/z=555.4 [M+H⁺]

To a solution of tert-butyl 4-[4-[1-[5-(1,2,3,4-tetrahydro-1,7-naphthyridin-6-yl)-2-pyridyl]-4-piperidyl]piperazin-1-yl]benzoate (190 mg, 342.51 μmol, 1.0 eq) and 1-(3-iodo-1-tetrahydropyran-4-yl-6,7-dihydro-4H-pyrazolo[4,3-c]pyridin-5-yl)ethanone (193 mg, 513.77 μmol, 1.5 eq) in dioxane (10 mL) was added t-BuONa (2 M, 513.77 μL, 3.0 eq) and methanesulfonato(2-dicyclohexylphosphino-2,4,6-tri-1-propyl-1,1-biphenyl)(2-methylamino-1,1-biphenyl-2-yl)palladium(II) (30 mg, 34.25 μmol, 0.1 eq). The mixture was stirred at 90° C. for 16 h. The reaction mixture was quenched by addition water 50 mL, and then extracted with ethyl acetate (50 mL×3). The combined organic layers were washed with brine (50 mL×3), dried over anhydrous sodium sulfate, filtered and concentrated under reduced pressure to give a residue. The residue was purified by prep-TLC (SiO₂, dichloromethane:methanol=15:1). Compound tert-butyl 4-[4-[1-[5-[1-(5-acetyl-1-tetrahydropyran-4-yl-6,7-dihydro-4H-pyrazolo[4,3-c]pyridin-3-yl)-3,4-dihydro-2H-1,7-naphthyridin-6-yl]-2-pyridyl]-4-piperidyl]piperazin-1-yl]benzoate (100 mg, 124.69 μmol, 36% yield) was obtained as a yellow oil.

LC-MS: MS (ESI⁺): t_{R}=0.429 min, m/z=802.4[M+H⁺]

To a solution of tert-butyl 4-[4-[1-[5-[1-(5-acetyl-1-tetrahydropyran-4-yl-6,7-dihydro-4H-pyrazolo[4,3-c]pyridin-3-yl)-3,4-dihydro-2H-1,7-naphthyridin-6-yl]-2-pyridyl]-4-piperidyl]piperazin-1-yl]benzoate (100 mg, 124.69 μmol, 1.0 eq) in dichloromethane (3 mL) was added trifluoroacetic acid (4.61 g, 40.39 mmol, 3 mL, 323.91 eq). The mixture was stirred at 25° C. for 0.5 h. The reaction mixture was concentrated in vacuum to give a residue. The residue was used into next step directly. Compound 4-[4-[1-[5-[1-(5-acetyl-1-tetrahydropyran-4-yl-6,7-dihydro-4H-pyrazolo[4,3-c]pyridin-3-yl)-3,4-dihydro-2H-1,7-naphthyridin-6-yl]-2-pyridyl]-4-piperidyl]piperazin-1-yl]benzoic acid (100 mg) was obtained as a yellow oil and directly used in the next step without further purification.

LC-MS: MS (ESI⁺): t_{R}=0.390 min, m/z=746.3 [M+1]

To a solution of 4-[4-[1-[5-[1-(5-acetyl-1-tetrahydropyran-4-yl-6,7-dihydro-4H-pyrazolo[4,3-c]pyridin-3-yl)-3,4-dihydro-2H-1,7-naphthyridin-6-yl]-2-pyridyl]-4-piperidyl]piperazin-1-yl]benzoic acid (100 mg) and 4-(3-amino-2,2,4,4-tetramethyl-cyclobutoxy)-2-methoxy-benzonitrile (73 mg, 232.58 μmol, 2.0 eq, HCl) in N,N-dimethylformamide (5 mL) was added O-(7-azabenzotriazol-1-yl)-N,N,N′,N′-tetramethyluronium hexafluorophosphate (133 mg, 348.86 μmol, 3.0 eq) and N,N-diisopropylethylamine (45 mg, 348.86 μmol, 60.77 μL, 3.0 eq). The mixture was stirred at 25° C. for 16 h. The reaction mixture was quenched by addition water 50 mL, and then extracted with ethyl acetate (50 mL×3). The combined organic layers were washed with brine (50 mL×3), dried over anhydrous sodium sulfate, filtered and concentrated under reduced pressure to give a residue. The residue was purified by prep-HPLC (column: Unisil 3-100 C18 Ultra 150*50 mm*3 urn; mobile phase: [water(FA)-ACN]; gradient: 15%-45% B over 10 mi). Compound 4-[4-[1-[5-[1-(5-acetyl-1-tetrahydroran-4-yl-6,7-dihydro-4H-pyrazolo[4,3-c]pyridin-3-yl)-3,4-dihydro-2H-1,7-naphthyridin-6-yl]-2-pyridyl]-4-piperidyl]piperazin-1-yl]-N-[3-(4-cyano-3-methoxy-phenoxy)-2,2,4,4-tetramethyl-cyclobutyl]benzamide (43.59 mg, 41.78 mol, 3600 yield over two steps) was obtained as an off-white solid).

¹H NMR (400 MHz, CHLOROFORM-d) δ=8.65 (dd, J=2.4, 7.3 Hz, 1H), 8.50 (s, 1H), 8.03 (ddd, J=2.4, 8.8, 10.8 Hz, 1H), 7.88 (s, 1H), 7.70 (d, J=8.8 Hz, 2H), 7.45 (d, J=8.8 Hz, 1H), 7.35-7.27 (m, 1H), 6.92 (d, J=8.8 Hz, 2H), 6.73 (dd, J=4.8, 9.0 Hz, 1H), 6.47 (d, J=2.0 Hz, 1H), 6.40 (dd, J=2.4, 8.6 Hz, 1H), 6.12 (d, J=8.0 Hz, 1H), 4.43 (d, J=13.2 Hz, 2H), 4.34 (s, 1H), 4.20-4.09 (m, 5H), 4.05 (s, 1H), 3.92 (s, 4H), 3.79-3.65 (m, 3H), 3.52 (t, J=12.4 Hz, 2H), 3.36-3.27 (m, 4H), 2.95-2.86 (m, 4H), 2.82 (t, J=5.6 Hz, 1H), 2.79-2.72 (m, 5H), 2.63-2.53 (m, 1H), 2.37-2.25 (m, 2H), 2.18-2.05 (m, 5H), 1.97 (d, J=12.4 Hz, 2H), 1.86 (d, J=13.2 Hz, 2H), 1.67-1.56 (m, 2H), 1.25 (d, J=9.6 Hz, 12H)

LC-MS: MS (ESI⁺): t_{R}=2.583 min, m/z=1002.8 [M+H⁺]

The compounds below were prepared in a similar manner as described in Example 43.

| | | | LC-MS |
|---|---|---|---|
| Compound | Name | ¹H NMR (CDCl₃) | [M + H⁺] |
| 324 | 4-[4-[1-[5-[1-(5-acetyl-1- | δ = 8.61 (dd, J = 7.8, 2.1 Hz, 1H), | 974.7 |
| | tetrahydropyran-4-yl-6,7-dihydro-4H- | 8.19 (s, 1H), 8.11-7.96 (m, 1H), 7.88 | |
| | pyrazolo[4,3-c]pyridin-3-yl)-3,4- | (s, 1H), 7.71 (d, J = 8.8 Hz, 2H), 7.45 | |
| | dihydro-2H-1,7-naphthyridin-6-yl]-2- | (d, J = 8.6 Hz, 1H), 7.28 (s, 1H), 7.25 | |
| | pyridyl]azetidin-3-yl]piperazin-1-yl]- | (s, 1H), 6.93 (d, J = 9.0 Hz, 2H), 6.47 | |
| | N-[3-(4-cyano-3-methoxy-phenoxy)- | (d, J = 2.1 Hz, 1H), 6.44-6.33 (m, | |
| | 2,2,4,4-tetramethyl- | 2H), 6.13 (d, J = 8. 1 Hz, 1H), 4.34 (s, | |
| | cyclobutyl]benzamide | 1H), 4.22-4.15 (m, 4H), 4.15-4.09 (m, | |
| | | 3H), 4.05 (s, 1H), 3.99 (dd, J = 7.9, | |
| | | 5.4 Hz, 2H), 3.92 (s, 4H), 3.78-3.66 | |
| | | (m, 3H), 3.52 (t, J = 12.0 Hz, 2H), | |
| | | 3.41 (t, J = 6.3 Hz, 1H), 3.39-3.32 | |
| | | (m, 4H), 2.95-2.85 (m, 2H), 2.82 (t, | |
| | | J = 5.4 Hz, 1H), 2.75 (t, J = 5.4 Hz, | |
| | | 1H), 2.67-2.56 (m, 4H), 2.36-2.24 (m, | |
| | | 2H), 2.17 (s, 1H), 2.15-2.03 (m, 4H), | |
| | | 1.84 (s, 1H), 1.25 (d, J = 9.6 Hz, | |
| | | 12H) | |
| 325 | 4-[4-[1-[5-[1-(5-acetyl-1- | (DMSO-d₆) δ = 8.04 (s, 1H), 7.86- | 1051.8 |
| | tetrahydropyran-4-yl-6,7-dihydro-4H- | 7.73 (m, 2H), 7.68-7.62 (m, 1H), | |
| | pyrazolo[4,3-c] pyridin-3-yl)-7- | 7.60-7.44 (m, 2H), 7.11-6.90 (m, | |
| | (difluoromethyl)-3,4-dihydro-2H- | 4H), 6.89-6.82 (m, 1H), 6.80-6.48 | |
| | quinolin-6-yl]-2-pyridyl]-4-piperidyl] | (m, 3H), 4.58-4.39 (m, 2H), 4.34- | |
| | piperazin-1-yl]-N-[3-(4-cyano-3- | 4.26 (m, 2H), 4.22-4.13 (m, 2H), | |
| | methoxy-phenoxy)-2,2,4,4- | 4.11-4.02 (m, 1H), 4.02-3.82 (m, | |
| | tetramethyl-cyclobutyl]benzamide | 6H), 3.79-3.58 (m, 5H), 3.54-3.39 | |
| | | (m, 4H), 3.27-3.02 (m, 4H), 2.93- | |
| | | 2.72 (m, 7H), 2.21-1.91 (m, 9H), | |
| | | 1.90-1.76 (m, 2H), 1.69-1.42 (m, | |
| | | 2H), 1.22 (s, 6H), 1.15 (s, 6H). | |
| 326 | 4-(4-(1-(5-(1-(5-acetyl-1-(tetrahydro- | δ = 7.99 (d, J = 2.0 Hz, 1H), 7.81- | 1023.4 |
| | 2H-pyran-4-yl)-4,5,6,7-tetrahydro- | 7.73 (m, 2H), 7.68-7.61 (m, 1H), | |
| | 1H-pyrazolo[4,3-c]pyridin-3-yl)-7- | 7.53 (d, J = 9.2 Hz, 1H), 7.50-7.43 | |
| | (difluoromethyl)-1,2,3,4- | (m, 1H), 7.05-6.94 (m, 3H), 6.89- | |
| | tetrahydroquinolin-6-yl)pyridin-2- | 6.81 (m, 1H), 6.78-6.49 (m, 3H), | |
| | yl)azetidin-3-yl)piperazin-1-yl)-N- | 6.48-6.44 (m, 1H), 4.35-4.25 (m, | |
| | ((1r,3r)-3-(4-cyano-3- | 2H), 4.21-4.12 (m, 2H), 4.10-4.02 | |
| | methoxyphenoxy)-2,2,4,4- | (m, 3H), 3.95 (d, J = 10.8 Hz, 2H), | |
| | tetramethylcyclobutyl)benzamide | 3.90 (s, 3H), 3.84 (d, J = 5.2, 7.6 Hz, | |
| | | 2H), 3.77-3.68 (m, 2H), 3.63-3.58 | |
| | | (m, 2H), 3.49-3.42 (m, 3H), 3.30 (d, | |
| | | J = 3.2 Hz, 8H), 2.87 (d, J = 6.4 Hz, | |
| | | 3H), 2.76 (d, J = 5.2 Hz, 1H), 2.08 (s, | |
| | | 2H), 2.04-1.93 (m, 5H), 1.87-1.77 | |
| | | (m, 2H), 1.22 (s, 6H), 1.14 (s, 6H) | |

### Example 44: Synthesis of 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]-N-[3-(4-cyano-3-methoxy-phenoxy)-2,2,4,4-tetramethyl-cyclobutyl]benzamide (Compound 277)

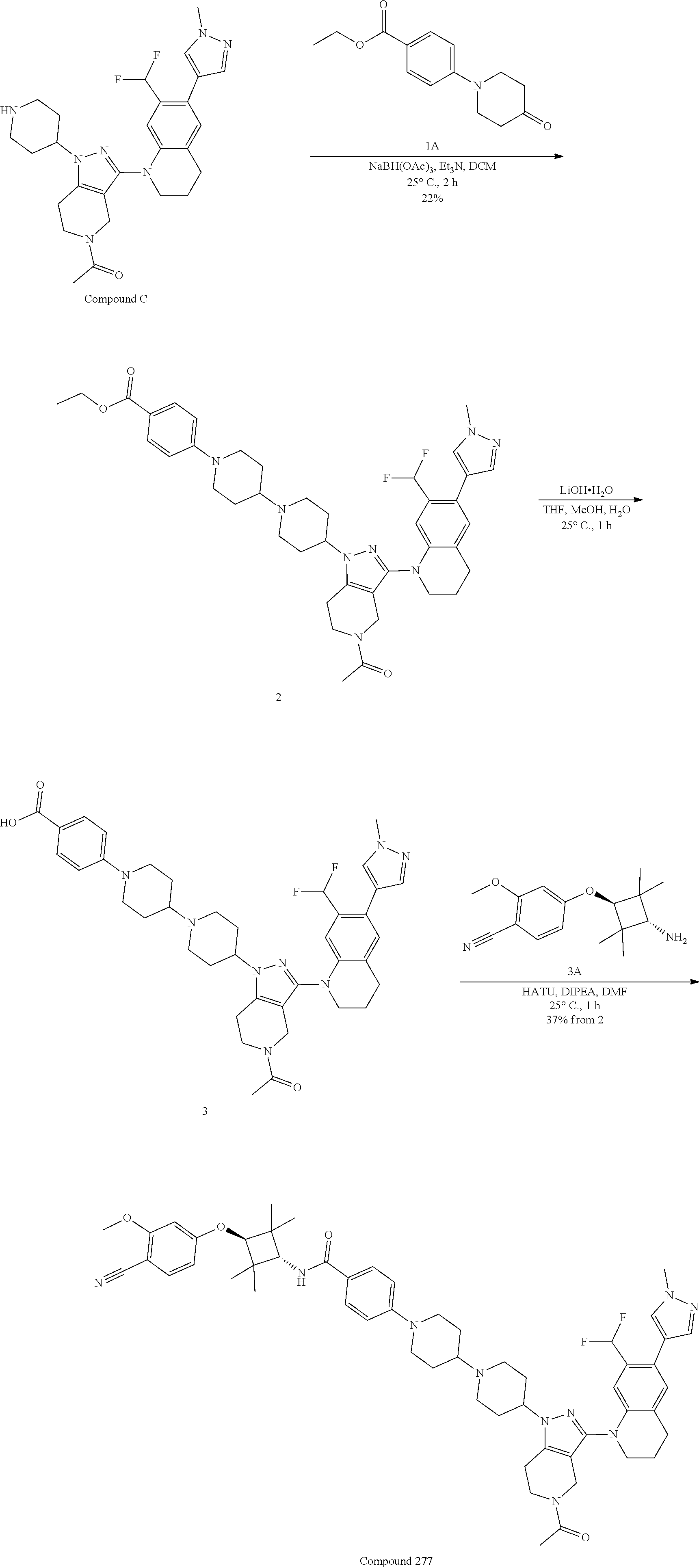

Synthesis of 1A was reported in European Journal of Medicinal Chemistry, 2013, vol. 66, p. 32-45.

To a solution of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (180 mg, 353 μmol, 1.0 eq), ethyl 4-(4-oxo-1-piperidyl)benzoate (530 μmol, 1.5 eq) in DCM (5 mL) was added Et₃N (107 mg, 1.0 mmol, 0.1 mL, 3.0 eq) and NaBH(OAc)₃ (370 mg, 1.77 mmol, 5.0 eq). The mixture was stirred at 25° C. for 2 h. The reaction mixture was quenched by addition of saturated NaHCO₃ (20 mL) at 0° C., and then extracted with ethyl acetate (30 mL×3) The combined organic layers were concentrated under reduce pressure to give a residue. The residue was purified by prep-HPLC (column: Welch Xtimate C18 150*25 mm*5 um; mobile phase: [water(FA)-ACN]; gradient: 20%-50% B over 10 min). Compound ethyl 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]benzoate (60 mg, 80.0 μmol, 22% over two steps) was obtained as a white solid.

LC-MS: MS (ESI⁺): t_{R}=0.513 min, m/z=741.4 [M+H⁺]

To a solution of ethyl 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]benzoate (50 mg, 67.4 μmol, 1.0 eq) in THF (0.5 mL) and MeOH (0.5 mL) was added the solution of LiOH H₂O (14 mg, 5.0 eq) in H₂O (0.5 mL). The mixture was stirred at 25° C. for 1 h. The reaction was acidified with aqueous 1 M HCl till pH=5, and then extracted with DCM (10 mL×3). The combined organic layers were dried, filtered, and concentrated under reduce pressure. Compound 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]benzoic acid (45 mg) was obtained as a white solid.

LC-MS: MS (ESI⁺): t_{R}=0.476 min, m/z=713.4 [M+H⁺]

To a solution of 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]benzoic acid (40 mg) in DMF (2 mL) was added DIPEA (36 mg, 281 μmol, 49 μL, 5.0 eq), HATU (43 mg, 112.23 μmol, 2.0 eq), and 4-(3-amino-2,2,4,4-tetramethyl-cyclobutoxy)-2-methoxy-benzonitrile (19 mg, 61.73 μmol, 1.1 eq, HCl salt). The mixture was stirred at 25° C. for 1 h. The reaction mixture was concentrated under reduced pressure to give the crude product. The residue was purified by prep-HPLC (column: Welch Xtimate C18 150*25 mm*5 um; mobile phase: [water(FA)-ACN]; gradient: 28%-58% B over 10 min). Compound 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]-N-[3-(4-cyano-3-methoxy-phenoxy)-2,2,4,4-tetramethyl-cyclobutyl]benzamide (20.36 mg, 20.17 μmol, 37% over two steps) was obtained as a yellow solid.

¹H NMR (400 MHz, CD₃OD) δ=7.76 (d, J=8.8 Hz, 2H), 7.64 (s, 1H), 7.55-7.44 (m, 3H), 7.13 (br d, J=8.4 Hz, 1H), 7.03 (br d, J=8.4 Hz, 2H), 6.77 (s, 1H), 6.68-6.51 (m, 3H), 4.53-4.35 (m, 1H), 4.28-4.22 (m, 3H), 4.16-4.12 (m, 1H), 4.11-4.08 (m, 3H), 4.08-4.05 (in, H), 3.93 (s, 3H), 3.92 (d, J=1.6 Hz, 3H), 3.91-3.88 (m, 1H), 3.87-3.81 (m, 1H), 3.79-3.63 (m, 4H), 3.26-3.14 (m, 2H), 2.96-2.82 (m, 6H), 2.44-2.32 (m, 2H), 2.31-2.23 (m, 2H), 2.19 (s, 3H), 2.12-2.05 (m, 2H), 2.05-1.98 (m, 2H), 1.92-1.74 (m, 2H), 1.29 (s, 6H), 1.24 (s, 6H)

LC-MS: MS (ESI⁺): t_{R}=2.303 min, m/z=969.6 [M+H⁺]

The compounds below were prepared in a similar manner as described in Example 44.

| | | | LC-MS |
|---|---|---|---|
| Compound | Name | ¹H NMR (CDCl₃) | [M + H⁺] |
| 370 | 4-[3-[7-[5-acetyl-3-[7- | δ = 7.66 (d, J = 8.8 Hz, 2H), 7.54-7.39 | 981.7 |
| | (difluoromethyl)-6-(1- | (m, 3H), 7.06-6.85 (m, 2H), 6.69-6.38 | |
| | methylpyrazol-4-yl)-3,4- | (m, 5H), 6.09 (d, J = 8.1 Hz, 1H), 4.27- | |
| | dihydro-2H-quinolin-1-yl]-6,7- | 4.10 (m, 3H), 4.05 (s, 1H), 4.03-3.80 (m, | |
| | dihydro-4H-pyrazolo[4,3- | 11H), 3.79-3.59 (m, 6H), 3.19 (s, 2H), | |
| | c]pyridin-1-yl]-2- | 3.09 (s, 2H), 2.92-2.83 (m, 2H), 2.78 (t, | |
| | azaspiro[3.5]nonan-2- | J = 5.4 Hz, 1H), 2.72 (t, J = 5.6 Hz, 1H), | |
| | yl]azetidin-1-yl]-N-[3-(4- | 2.16 (s, 2H), 2.13 (s, 1H), 2. 10-2.03 (m, | |
| | cyano-3-methoxy-phenoxy)- | 4H), 2.02-1.93 (m, 2H), 1.93-1.86 (m, | |
| | 2,2,4,4-tetramethyl- | 2H), 1.25 (d, J = 9.9 Hz, 12H) | |
| | cyclobutyl]benzamide | | |
| 371 | 2-[4-[7-[5-acetyl-3-[7- | δ = 8.71 (s, 2H), 7.62-7.50 (m, 2H), 7.41 | 1015.7 |
| | (difluoromethyl)-6-(1- | (d, J = 5.6 Hz, 1H), 7.08-6.95 (m, 2H), | |
| | methylpyrazol-4-yl)-3,4- | 6.90-6.78 (m, 2H), 6.70-6.33 (m, 1H), | |
| | dihydro-2H-quinolin-1-yl]-6,7- | 6.21-6.00 (m, 1H), 4.89-4.81 (m, 2H), | |
| | dihydro-4H-pyrazolo[4,3- | 4.76 (d, J = 5.4 Hz, 8H), 4.26 (s, 1H), | |
| | c]pyridin-1-yl]-2- | 4.16-4.04 (m, 3H), 3.96 (s, 3H), 3.92- | |
| | azaspiro[3.5]nonan-2-yl]-1- | 3.81 (m, 2H), 3.78-3.65 (m, 3H), 3.47- | |
| | piperidyl]-N-[3-(3-chloro-4- | 3.21 (m, 3H), 3.16-3.04 (m, 2H), 2.94- | |
| | cyano-phenoxy)-2,2,4,4- | 2.84 (m, 2H), 2.81-2.67 (m, 2H), 2.16 | |
| | tetramethyl- | (s, 1H), 2.11-2.03 (m, 4H), 1.95-1.81 | |
| | cyclobutyl]pyrimidine-5- | (m, 6H), 1.24 (d, J = 17.2 Hz, 12H) | |
| | carboxamide | | |
| 372 | 4-[4-[7-[5-acetyl-3-[7- | δ = 8.87-8.67 (m, 1H), 7.67-7.57 (m, | 1009.7 |
| | (difluoromethyl)-6-(1- | 2H), 7.54-7.49 (m, 1H), 7.46-7.40 (m, | |
| | methylpyrazol-4-yl)-3,4- | 2H), 7.08-6.99 (m, 1H), 6.94-6.82 (m, | |
| | dihydro-2H-quinolin-1-yl]-6,7- | 3H), 6.60-6.28 (m, 4H), 4.30-4.20 (m, | |
| | dihydro-4H-pyrazolo[4,3- | 1H), 4.17-4.04 (m, 4H), 4.02-3.81 (m, | |
| | c]pyridin-1-yl]-2- | 13H), 3.76-3.61 (m, 3H), 3.31 (d, J = | |
| | azaspiro[3.5]nonan-2-yl]-1- | 10.8 Hz, 1H), 2.93-2.62 (m, 6H), 2.37- | |
| | piperidyl]-N-[3-(4-cyano-3- | 2.25 (m, 1H), 2.18-2.00 (m, 8H), 1.98- | |
| | methoxy-phenoxy)-2,2,4,4- | 1.82 (m, 4H), 1.78-1.67 (m, 4H), 1.23 | |
| | tetramethyl- | (d, J = 16.8 Hz, 12H) | |
| | cyclobutyl]benzamide | | |
| 373 | 2-[4-[2-[5-acetyl-3-[7- | δ = 8.70 (s, 2 H) 7.57 (d, J = 8.8 Hz, 1 H) | 1015.7 |
| | (difluoromethyl)-6-(1- | 7.53 (d, J = 5.2 Hz, 1 H) 7.41 (d, J = 6.4 | |
| | methylpyrazol-4-yl)-3,4- | Hz, 1 H) 7.07-7.00 (m, 1 H) 6.99-6.94 | |
| | dihydro-2H-quinolin-1-yl]-6,7- | (m, 2 H) 6.81 (dd, J = 8.8, 2.4 Hz, 1 H) | |
| | dihydro-4H-pyrazolo[4,3- | 6.69-6.37 (m, 1 H) 5.96 (d, J = 8.0 Hz, 1 | |
| | c]pyridin-1-yl]-7- | H) 4.97 (d, J = 13.2 Hz, 2 H) 4.62-4.50 | |
| | azaspiro[3.5]nonan-7-yl]-1- | (m, 1 H) 4.25 (s, 1 H) 4.15-4.10 (m, 2 H) | |
| | piperidyl]-N-[3-(3-chloro-4- | 4.05 (s, 1 H) 3.96 (d, J = 2.4 Hz, 3 H) | |
| | cyano-phenoxy)-2,2,4,4- | 3.91-3.86 (m, 1 H) 3.73 (d, J = 5.6 Hz, 2 | |
| | tetramethyl- | H) 3.00-2.92 (m, 2 H) 2.92-2.85 (m, 3 H) | |
| | cyclobutyl]pyrimidine-5- | 2.75 (d, J = 4.8 Hz, 3 H) 2.70-2.61 (m, 3 | |
| | carboxamide | H) 2.55-2.42 (m, 3 H) 2.32 (d, J = 10.4 | |
| | | Hz, 3 H) 2.15 (s, 1 H) 2.11-2.05 (m, 3 H) | |
| | | 2.04 (s, 2 H) 1.88 (d, J = 5.2 Hz, 4 H) | |
| | | 1.58 (qd, J = 12.0, 3.6 Hz, 2 H) 1.26 (s, 6 | |
| | | H) 1.22 (s, 6 H) | |
| 374 | 4-[4-[2-[5-acetyl-3-[7- | δ = 7.67 (d, J = 8.5 Hz, 2H), 7.53 (d, J = | 1009.7 |
| | (difluoromethyl)-6-(1- | 6.0 Hz, 1H), 7.47-7.40 (m, 2H), 7.07 (d, | |
| | methylpyrazol-4-yl)-3,4- | J = 5.5 Hz, 1H), 7.03-6.97 (m, 1H), | |
| | dihydro-2H-quinolin-1-yl]-6,7- | 6.92-6.84 (m, 2H), 6.76-6.44 (m, 2H), | |
| | dihydro-4H-pyrazolo[4,3- | 6.40 (dd, J = 1.9, 8.6 Hz, 1H), 6.34-6.28 | |
| | c]pyridin-1-yl]-7- | (m, 1H), 4.71-4.52 (m, 1H), 4.34-4.23 | |
| | azaspiro[3.5]nonan-7-yl]-1- | (m, 1H), 4.17-4.06 (m, 3H), 4.04-3.82 | |
| | piperidyl]-N-[3-(4-cyano-3- | (m, 9H), 3.76-3.66 (m, 3H), 3.58-3.30 | |
| | methoxy-phenoxy)-2,2,4,4- | (m, 3H), 3.09-2.79 (m, 6H), 2.78-2.71 | |
| | tetramethyl- | (m, 1H), 2.69-2.64 (m, 1H), 2.61-2.33 | |
| | cyclobutyl]benzamide | (m, 4H), 2.30-2.12 (m, 5H), 2.12-2.02 | |
| | | (m, 6H), 1.90-1.80 (m, 2H), 1.24 (d, J = | |
| | | 15.3 Hz, 12H) | |

### Example 45: Synthesis of 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-methyl-amino]-1-piperidyl]-N-[3-(4-cyano-3-methoxy-phenoxy)-2,2,4,4-tetramethyl-cyclobutyl]benzamidine (Compound 278)

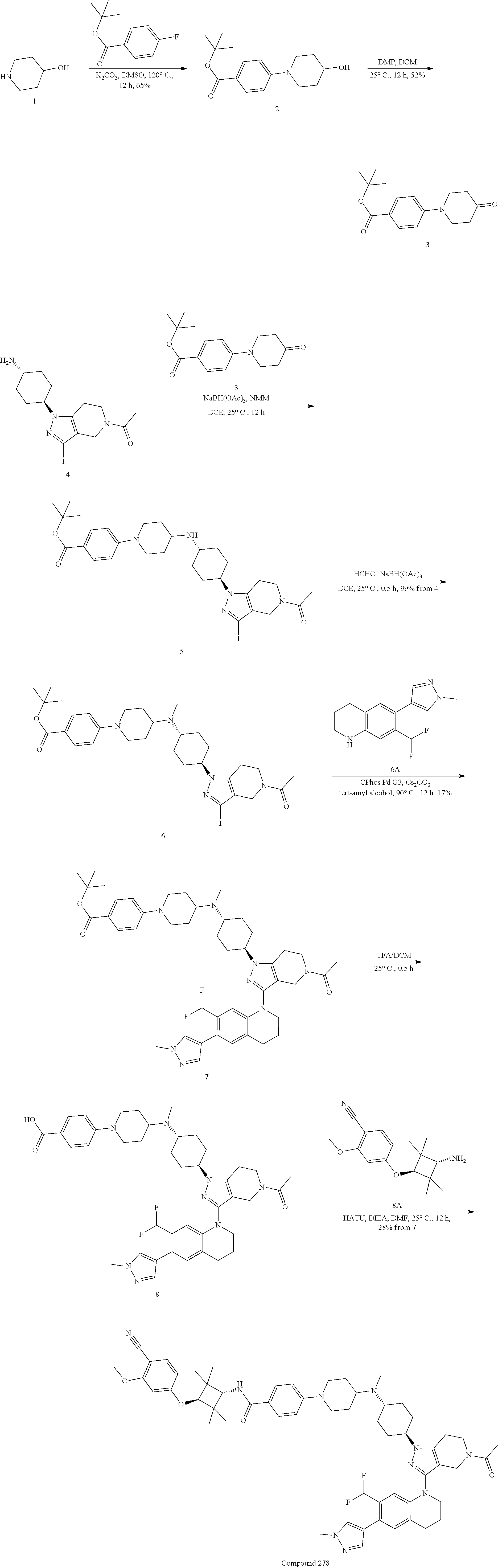

To a solution of tert-butyl 4-fluorobenzoate (10.00 g, 50.96 mmol, 1.0 eq) in DMSO (50 mL) was added K₂CO₃ (17.61 g, 127.41 mmol, 2.5 eq) and piperidin-4-ol (5.15 g, 50.96 mmol, 1.0 eq). The mixture was stirred at 120° C. for 12 hours. The mixture was diluted with water (200 mL) and extracted with ethyl acetate (80 mL×3). The combined organic layers were washed with brine (300 mL×3), dried over anhydrous sodium sulfate, filtered and concentrated in vacuum. The residue was purified by silica gel chromatography (0%-40% ethyl acetate in petroleum ether). Compound tert-butyl 4-(4-hydroxy-1-piperidyl)benzoate (9.24 g, 33.31 mmol, 65% yield) was obtained as a white solid.

¹H NMR (400 MHz, CDCl₃) δ=7.96-7.77 (m, 2H), 6.87 (d, J=9.0 Hz, 2H), 3.97-3.85 (m, 1H), 3.80-3.64 (m, 2H), 3.07 (dd, J=13.0, 9.8 Hz, 2H), 2.07-1.91 (m, 2H), 1.69-1.56 (m, 12H)

To a solution of tert-butyl 4-(4-hydroxy-1-piperidyl)benzoate (5.24 g, 18.89 mmol, 1.0 eq) in dichloromethane (50 mL) was added Dess-Martin (10.42 g, 24.56 mmol, 7.61 mL, 1.3 eq). The mixture was stirred at 25° C. for 12 h. The mixture was diluted with water (200 mL) and extracted with ethyl acetate (80 mL×3). The combined organic layers were washed with brine (300 mL×3), dried over anhydrous sodium sulfate, filtered and concentrated in vacuum. The residue was purified by silica gel chromatography (0%-10% ethyl acetate in petroleum ether). Compound tert-butyl 4-(4-oxo-1-piperidyl)benzoate (2.73 g, 9.91 mmol, 52% yield) was obtained as a yellow solid.

¹H NMR (400 MHz, CDCl₃) δ=7.92 (d, J=9.0 Hz, 2H), 6.90 (d, J=9.0 Hz, 2H), 3.74 (t, J=6.1 Hz, 4H), 2.56 (t, J=6.1 Hz, 4H), 1.59 (s, 9H)

LC-MS: MS (ESI⁺: t_{R}=0.533 min, m/z=276.0 [M+H⁺]

To a solution of 1-[1-(4-aminocyclohexyl)-3-iodo-6,7-dihydro-4H-pyrazolo[4,3-c]pyridin-5-yl]ethanone (357 mg, 919.52 μmol, 1.0 eq) in DCE (5 mL) was added NMM (930 mg, 9.20 mmol, 1.01 mL, 10.0 eq) and tert-butyl 4-(4-oxo-1-piperidyl)benzoate (379 mg, 1.38 mmol, 1.5 eq). The mixture was stirred at 25° C. for 10 min. NaBH(OAc)₃ (584 mg, 2.76 mmol, 3.0 eq) was added and the mixture was stirred at 25° C. for 12 h. The mixture was poured into water (50 mL). The aqueous phase was extracted with ethyl acetate (20 mL×3). The combined organic phase was washed with brine (100 mL×3), dried with anhydrous sodium sulfate, filtered and concentrated in vacuum. Compound tert-butyl 4-[4-[[4-(5-acetyl-3-iodo-6,7-dihydro-4H-pyrazolo[4,3-c]pyridin-1-yl)cyclohexyl]amino]-1-piperidyl]benzoate (595 mg, 918.79 μmol, 99% yield) was obtained as a yellow oil and directly used in the next step.

LC-MS: MS (ESI⁺: t_{R}=0.488 min, m/z=648.3 [M+H⁺]

To a solution of tert-butyl 4-[4-[[4-(5-acetyl-3-iodo-6,7-dihydro-4H-pyrazolo[4,3-c]pyridin-1-yl)cyclohexyl]amino]-1-piperidyl]benzoate (595 mg, 918.79 μmol, 1.0 eq) in DCE (8 mL) was added HCHO (137 mg, 4.59 mmol, 126.57 μL, 5.0 eq) and NaBH(OAc)₃ (584 mg, 2.76 mmol, 3.0 eq). The mixture was stirred at 25° C. for 0.5 h. The mixture was poured into water (50 mL). The aqueous phase was extracted with dichloromethane (30 mL×2). The combined organic phase was washed with brine (80 mL×3), dried with anhydrous sodium sulfate, filtered and concentrated in vacuum. The residue was purified by silica gel chromatography (0%-10% methanol in dichloromethane). Compound tert-butyl 4-[4-[[4-(5-acetyl-3-iodo-6,7-dihydro-4H-pyrazolo[4,3-c]pyridin-1-yl)cyclohexyl]-methyl-amino]-1-piperidyl]benzoate (600 mg, 906.87 μmol, 98% yield) was obtained as a white solid.

¹H NMR (400 MHz, CDCl₃) δ=7.87 (d, J=8.9 Hz, 2H), 6.86 (d, J=9.0 Hz, 2H), 4.25 (s, 1H), 3.95-3.86 (m, 4H), 2.90-2.62 (m, 6H), 2.30 (s, 3H), 2.22-2.16 (m, 3H), 2.13-1.94 (m, 8H), 1.87 (d, J=12.0 Hz, 2H), 1.75-1.65 (m, 2H), 1.58 (s, 9H), 1.57-1.44 (m, 2H)

LC-MS: MS (ESI: t_{R}=0.497 min, m/z=662.3 [M+H⁺]

To a solution of 7-(difluoromethyl)-6-(1-methylpyrazol-4-yl)-1,2,3,4-tetrahydroquinoline (178 mg, 676.07 μmol, 1.0 eq), tert-butyl 4-[4-[[4-(5-acetyl-3-iodo-6,7-dihydro-4H-pyrazolo[4,3-c]pyridin-1-yl)cyclohexyl]-methyl-amino]-1-piperidyl]benzoate (447 mg, 676.07 μmol, 1.0 eq) in tert-amyl alcohol (8 mL) was added Cs₂CO₃ (660 mg, 2.03 mmol, 3.0 eq) and Cphos Pd G3 (54 mg, 67.61 μmol, 0.1 eq). The mixture was stirred at 90° C. for 12 h under N₂. The mixture was poured into water (50 mL). The aqueous phase was extracted with ethyl acetate (30 mL×2). The combined organic phase was washed with brine (50 mL×3), dried with anhydrous sodium sulfate, filtered and concentrated in vacuum. The residue was purified by prep-TLC (SiO₂, Dichloromethane:Methanol=10:1). The residue was purified by prep-HPLC (column: Phenomenex luna C18 150*25 mm*10 um; mobile phase: [water (FA)-ACN]; gradient: 20%-50% B over 10 min) to give desired compound. Then it was lyophilized. Compound tert-butyl 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-methyl-amino]-1-piperidyl]benzoate (96 mg, 120.45 μmol, 17% yield) was obtained as a white solid.

LC-MS: MS (ESI⁺): t_{R}=0.505 min, m/z=797.3 [M+H⁺]

To a solution of tert-butyl 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-methyl-amino]-1-piperidyl]benzoate (96 mg, 120.45 μmol, 1.0 eq) in dichloromethane (3 mL) was added trifluoroacetic acid (4.61 g, 40.39 mmol, 3 mL, 335.30 eq). The mixture was stirred at 25° C. for 0.5 h. The reaction mixture was concentrated under reduced pressure to give the product. Compound 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-methyl-amino]-1-piperidyl]benzoic acid (89 mg) was obtained as a yellow oil and directly used in the next step without further purification.

LC-MS: MS (ESI⁺): t_{R}=0.455 min, m/z=741.4 [M+H⁺]

To a solution of 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-methyl-amino]-1-piperidyl]benzoic acid (89 mg) in DMF (3 mL) was added DIPEA (310 mg, 2.40 mmol, 418.47 μL, 20.0 eq), 4-(3-amino-2,2,4,4-tetramethyl-cyclobutoxy)-2-methoxy-benzonitrile (37 mg, 120.13 μmol, 1.0 eq) and the mixture was stirred at 25° C. for 10 min. hexafluorophosphate azabenzotriazole tetramethyl uronium (91 mg, 240.25 μmol, 2.0 eq) was added and the mixture was stirred at 25° C. for 12 h. The mixture was poured into water (50 mL). The aqueous phase was extracted with ethyl acetate (30 mL×2). The combined organic phase was washed with brine (50 mL×3), dried with anhydrous sodium sulfate, filtered and concentrated in vacuum. The residue was purified by prep-TLC (SiO₂, Dichloromethane:Methanol=15:1). The residue was purified by prep-HPLC (column: Phenomenex luna C18 150*25 mm*10 um; mobile phase: [water (FA)-ACN]; gradient: 230%-533% B over 10 min) to give desired compound. Then it was lyophilized. Compound 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-methyl-amino]-1-piperidyl]-N-[3-(4-cyano-3-methoxy-phenoxy)-2,2,4,4-tetramethyl-cyclobutyl]benzamide (35.07 mg, 34.82 μmol, 28% yield) was obtained as a white solid

¹H NMR (400 MHz, CDCl₃) δ=8.48 (s, 1H), 7.69 (d, J=8.8 Hz, 2H), 7.54 (d, J=5.4 Hz, 1H), 7.46 (d, J=8.6 Hz, 1H), 7.41 (d, J=6.5 Hz, 1H), 7.05 (s, 1H), 6.92 (d, J=8.9 Hz, 2H), 6.87 (d, J=5.4 Hz, 1H), 6.68-6.37 (m, 3H), 6.12 (d, J=8.4 Hz, 1H), 4.30-4.10 (m, 3H), 4.05 (s, 1H), 4.00-3.84 (n, 10H), 3.80-3.62 (m, 3H), 2.92-2.79 (m, 6H), 2.79-2.70 (m, 2H), 2.40-2.27 (m, 3H), 2.17 (s, 1H), 2.13-1.97 (m, 10H), 1.91 (d, J=12.5 Hz, 2H), 1.70-1.49 (m, 4H), 1.25 (d, J=10.5 Hz, 12H)

LC-MS: MS (ESI⁺): t_{R}=3.034 min, m/z=997.8 [M+H⁺]

The compounds below were prepared in a similar manner as described in Example 45.

| | | | LC-MS |
|---|---|---|---|
| Compound | Name | ¹H NMR (CDCl₃) | [M + H⁺] |
| 280 | 4-[4-[[4-[5-acetyl-3-[7- | δ = 9.73 (d, J = 8.8 Hz, 1H), 7.96 (d, J = | 997.7 |
| | (difluoromethyl)-6-(1- | 9.6 Hz, 1H), 7.87 (d, J = 8.0 Hz, 1H), | |
| | methylpyrazol-4-yl)-3,4- | 7.79-7.62 (m, 2H), 7.60-7.44 (m, 3H), | |
| | dihydro-2H-quinolin-1-yl]-6,7- | 7.06-6.94 (m, 2H), 6.85 (dd, J = 2.4, 8.8 | |
| | dihydro-4H-pyrazolo[4,3- | Hz, 1H), 4.66 (s, 1H), 4.69-4.60 (m, | |
| | c]pyridin-1-yl]cyclohexyl]- | 1H), 4.55-4.44 (m, 3H), 4.38-4.27 (m, | |
| | methyl-amino]-1-piperidyl]-N- | 1H), 4.11-3.96 (m, 3H), 3.83 (br t, J = | |
| | [3-(4-cyano-3-methoxy- | 5.6 Hz, 1H), 3.09-2.98 (m, 4H), 2.97- | |
| | phenoxy)-2,2,4,4-tetramethyl- | 2.79 (m, 3H), 2.71 (d, J = 6.0 Hz, 3H), | |
| | cyclobutyl]benzamide | 2.45-2.34 (m, 2H), 2.31-2.26 (m, 2H), | |
| | | 2.25-2.14 (m, 7H), 2.07 (s, 2H), 2.04- | |
| | | 1.92 (m, 4H), 1.76-1.61 (m, 2H), 1.53- | |
| | | 1.39 (m, 2H), 1.32-1.17 (m, 2H) | |
| 282 | 2-[4-[[4-[5-acetyl-3-[7- | δ = 8.75 (s, 2H), 8.32 (s, 1H), 7.95-7.84 | 1003.4 |
| | (difluoromethyl)-6-(1- | (m, 1H), 7.76-7.67 (m, 2H), 7.49 (s, | |
| | methylpyrazol-4-yl)-3,4- | 1H), 7.24-7.17 (m, 1H), 7.09 (s, 1H), | |
| | dihydro-2H-quinolin-1-yl]-6,7- | 7.04-6.96 (m, 1H), 6.94-6.62 (m, 2H), | |
| | dihydro-4H-pyrazolo[4,3-c] | 4.80-4.67 (m, 2H), 4.28 (s, 1H), 4.20- | |
| | pyridin-1-yl] cyclohexyl]- | 4.08 (m, 2H), 4.07-3.97 (m, 2H), 3.86 | |
| | methyl-amino]-1-piperidyl]-N- | (s, 3H), 3.76-3.54 (m, 5H), 3.08-2.93 | |
| | [3-(3-chloro-4-cyano-phenoxy)- | (m, 2H), 2.83 (s, 4H), 2.75-2.59 (m, | |
| | 2,2,4,4-tetramethyl-cyclobutyl] | 2H), 2.19 (s, 3H), 2.07 (s, 2H), 2.00- | |
| | pyrimidine-5-carboxamide | 1.87 (m, 6H), 1.84 (s, 4H), 1.56-1.34 | |
| | | (m, 4H), 1.21 (s, 6H), 1.10 (s, 6H). | |
| 284 | 2-[4-[[4-[5-acetyl-3-[7- | δ = 8.69 (s, 2H), 7.57 (d, J = 8.8 Hz, 1H), | 1003.6 |
| | (difluoromethyl)-6-(1- | 7.53 (d, J = 5.2 Hz, 1H), 7.41 (d, J = 6.4 | |
| | methylpyrazol-4-yl)-3,4- | Hz, 1H), 7.08-6.95 (m, 2H), 6.90 (d, J = | |
| | dihydro-2H-quinolin-1-yl]-6,7- | 8.4 Hz, 1H), 6.81 (dd, J = 2.4, 8.8 Hz, | |
| | dihydro-4H-pyrazolo[4,3- | 1H), 6.71-6.34 (m, 1H), 5.98 (d, J = 8.0 | |
| | c]pyridin-1-yl]cyclohexyl]- | Hz, 1H), 4.94 (d, J = 12.4 Hz, 2H), 4.28 | |
| | methyl-amino]-1-piperidyl]-N- | (s, 1H), 4.20-4.10 (m, 3H), 4.05 (s, 1H), | |
| | [3-(3-chloro-4-cyano-phenoxy)- | 3.96 (d, J = 2.4 Hz, 3H), 3.90 (t, J = 5.6 | |
| | 2,2,4,4-tetramethyl- | Hz, 1H), 3.81-3.66 (m, 3H), 3.15-3.02 | |
| | cyclobutyl]pyrimidine-5- | (m, 1H), 2.97-2.83 (m, 5H), 2.82-2.71 | |
| | carboxamide | (m, 2H), 2.32-2.22 (m, 6H), 2.17 (s, | |
| | | 1H), 2.13-1.99 (m, 4H), 1.89 (d, J = | |
| | | 12.4 Hz, 2H), 1.79 (s, 2H), 1.70-1.55 | |
| | | (m, 4H), 1.24 (d, J = 15.2 Hz, 12H) | |

### Example 46: Synthesis of 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-2,7-diazaspiro[3.5]nonan-2-yl]-N-[3-(4-cyano-3-methoxy-phenoxy)-2,2,4,4-tetramethyl-cyclobutyl]benzamide (Compound 288)

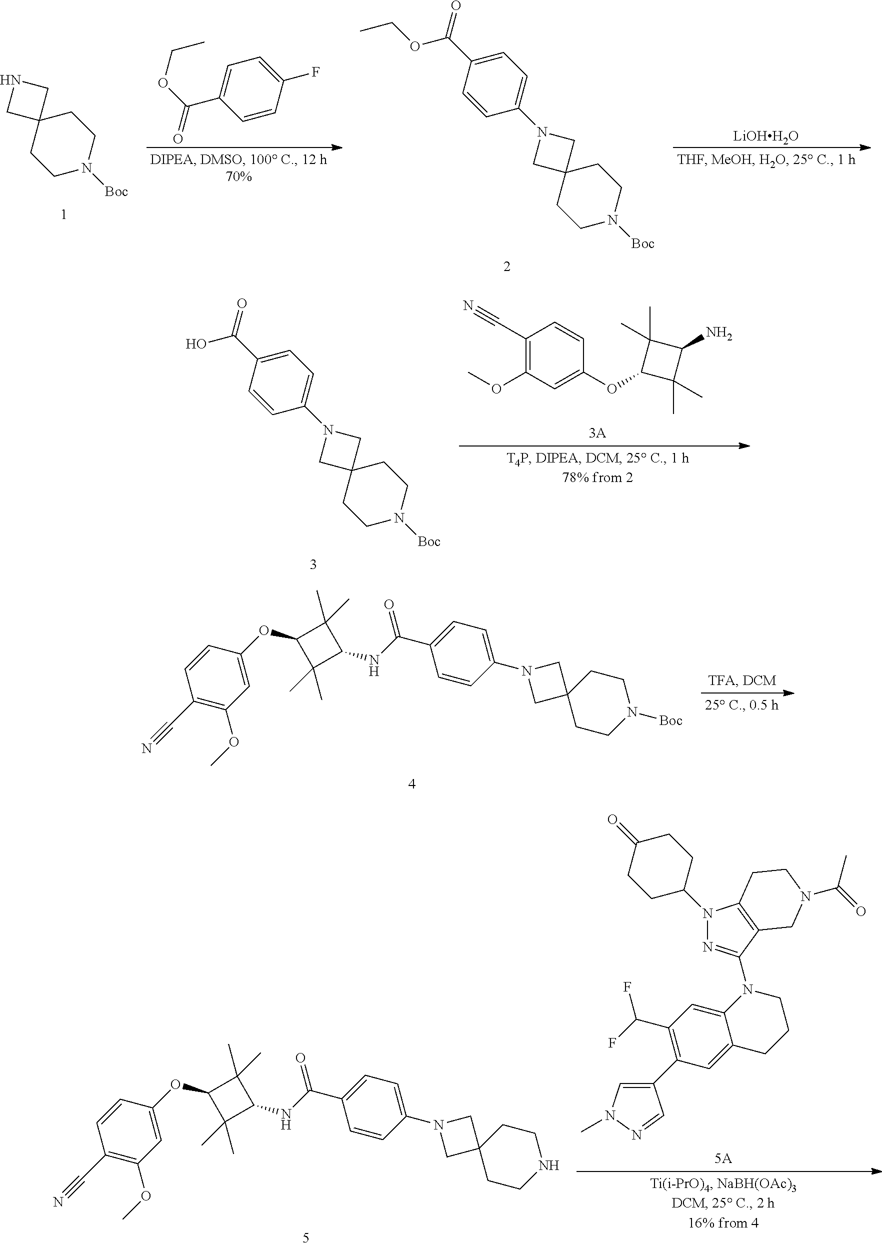

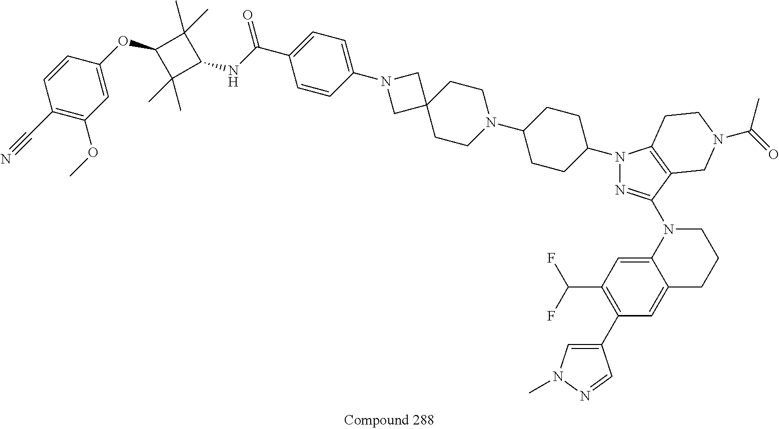 [SMILES: COc1cc(O[C@H]2C(C)(C)[C@H](NC(=O)c3ccc(N4CC5(CCN(C6CCC(n7nc(N8CCCc9cc(-c%10cnn(C)c%10)c(C(F)F)cc98)c8c7CCN(C(C)=O)C8)CC6)CC5)C4)cc3)C2(C)C)ccc1C#N]

Synthesis of 5A was reported in Journal of Medicinal Chemistry, 2024, vol. 67, #7, p. 5275-5304.

A mixture of tert-butyl 2,7-diazaspiro [3.5]nonane-7-carboxylate (5.00 g, 22.1 mmol, 1.0 eq), ethyl 4-fluorobenzoate (3.72 g, 22.1 mmol, 3.2 mL, 1.0 eq), DIPEA (8.57 g, 66.3 mmol, 11.5 mL, 3.0 eq) in DMSO (50 mL) was degassed and purged with N₂ for 3 times, and then the mixture was stirred at 100° C. for 12 h under N₂ atmosphere. The reaction mixture was quenched by addition water (500 mL), and then extracted with EtOAc (200 mL×3). The combined organic layers were washed with brine (500 mL×2), dried over Na₂SO₄, filtered and concentrated under reduced pressure to give a residue. The residue was purified by column chromatography (SiO₂, Petroleum ether/Ethyl acetate=1/0 to 3/1). Compound tert-butyl 2-(4-ethoxycarbonylphenyl)-2,7-diazaspiro [3.5]nonane-7-carboxylate (5.87 g, 15.7 mmol, 70% yield) was obtained as a yellow oil.

LC-MS: MS (ESI⁺: t_{R}=0.706 min, m/z=375.1 [M+H⁺]

To a solution of tert-butyl 2-(4-ethoxycarbonylphenyl)-2,7-diazaspiro [3.5]nonane-7-carboxylate (3.00 g, 8.01 mmol, 1.0 eq) in THF (20 mL), MeOH (20 mL) and H₂O (10 mL) was added LiOH·H₂O (3.36 g, 80.1 mmol, 10.0 eq). The mixture was stirred at 25° C. for 1 h. The reaction mixture was adjusted pH about 3 by HCl (1 M) and then extracted with EtOAc (100 mL×3). The combined organic layers were washed with brine (200 mL×2), dried over Na₂SO₄, filtered and concentrated under reduced pressure to give a residue. Compound 4-(7-tert-butoxycarbonyl-2,7-diazaspiro [3.5]nonan-2-yl) benzoic acid (2.78 g) was obtained as a white solid and directly used in the next step without further purification.

LC-MS: MS (ESI: t_{R}=0.535 min, m/z=247.0 [M−99]

To a solution of 4-(3-amino-2,2,4,4-tetramethyl-cyclobutoxy)-2-methoxy-benzonitrile (350 mg, 1.28 mmol, 1.0 eq) in DCM (10 mL) was added DIPEA (449 mg, 3.48 mmol, 606 μL, 3.0 eq) and T₄P (1.67 g, 2.32 mmol, 50% purity, 2.0 eq). Then 4-(7-tert-butoxycarbonyl-2,7-diazaspiro [3.5]nonan-2-yl) benzoic acid (401 mg) was added. The mixture was stirred at 25° C. for 1 h. The reaction mixture was quenched by addition water (60 mL), and then extracted with DCM (40 mL×3). The combined organic layers were washed with brine (70 mL×2), dried over Na₂SO₄, filtered and concentrated under reduced pressure to give a residue. The residue was purified by prep-TLC (SiO₂, Petroleum ether/Ethyl acetate=2/1). Compound tert-butyl 2-[4-[[3-(4-cyano-3-methoxy-phenoxy)-2,2,4,4-tetramethyl-cyclobutyl]carbamoyl]phenyl]-2,7-diazaspiro[3.5]nonane-7-carboxylate (550 mg, 912 μmol, 78% yield over two steps) was obtained as a white solid.

LC-MS: MS (ESI: t_{R}=0.624 min, m/z=603.4 [M+H⁺]

To a solution of tert-butyl 2-[4-[[3-(4-cyano-3-methoxy-phenoxy)-2, 2, 4, 4-tetramethyl-cyclobutyl]carbamoyl]phenyl]-2, 7-diazaspiro [3.5]nonane-7-carboxylate (150 mg, 248 μmol, 1.0 eq) in DCM (2 mL) was added TFA (2 mL). The mixture was stirred at 25° C. for 0.5 h. The reaction mixture was concentrated under reduced pressure to give a residue. Compound N-[3-(4-cyano-3-methoxy-phenoxy)-2, 2, 4, 4-tetramethyl-cyclobutyl]-4-(2, 7-diazaspiro [3.5]nonan-2-yl) benzamide (110 mg) was obtained as a white solid and directly used in the next step without further purification.

LC-MS: MS (ESI: t_{R}=0.521 min, m/z=503.3 [M+H⁺]

To a solution of 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexanone (50 mg, 95 μmol, 1.0 eq) and N-[3-(4-cyano-3-methoxy-phenoxy)-2,2,4,4-tetramethyl-cyclobutyl]-4-(2,7-diazaspiro[3.5]nonan-2-yl)benzamide (57 mg) in DCM (4 mL) was added Ti(i-PrO)₄ (54 mg, 191 μmol, 56 μL, 2.0 eq) at 25° C. for 1 h, then NaBH(OAc)₃ (101 mg, 478 μmol, 5.0 eq) was added. The mixture was stirred at 60° C. for 1 h. The reaction mixture was quenched by addition water (40 mL), and then extracted with DCM (30 mL×3). The combined organic layers were washed with brine (60 mL×2), dried over Na₂SO₄, filtered and concentrated under reduced pressure to give a residue. The residue was purified by prep-HPLC (column: Phenomenex Luna C18 150*25 mm*10 um; mobile phase: [H₂O (0.225% FA)-ACN]; gradient: 30%-60% B over 15.0 min). Compound 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-2,7-diazaspiro[3.5]nonan-2-yl]-N-[3-(4-cyano-3-methoxy-phenoxy)-2,2,4,4-tetramethyl-cyclobutyl]benzamide (16.10 mg, 15.95 μmol, 16% yield over two steps) was obtained as a white solid.

¹H NMR (400 MHz, DMSO-d₆) δ=8.13 (s, 1H), 7.78-7.71 (m, 3H), 7.64 (d, J=8.4 Hz, 1H), 7.49 (s, 1H), 7.43 (d, J=9.2 Hz, 1H), 7.11 (s, 1H), 6.82-6.61 (m, 3H), 6.53 (d, J=8.4 Hz, 1H), 6.41 (d, J=8.4 Hz, 2H), 4.45-4.31 (m, 1H), 4.27 (s, 1H), 4.21-4.11 (m, 2H), 4.04 (d, J=9.2 Hz, 1H), 3.90 (s, 3H), 3.84 (s, 3H), 3.75-3.58 (m, 9H), 3.35 (s, 3H), 2.85 (s, 4H), 2.73 (s, 1H), 2.23-2.10 (m, 4H), 2.08 (s, 3H), 2.03-1.93 (m, 6H), 1.92-1.76 (m, 4H), 1.21 (s, 6H), 1.14 (s, 6H).

LC-MS: MS (ESI⁺): t_{R}=2.515 min, m/z=1009.8 [M+H⁺]

The compounds below were prepared in a similar manner as described in Example 46.

| | | | LC-MS |
|---|---|---|---|
| Compound | Name | ¹H NMR (CDCl₃) | [M + H⁺] |
| 391 | 2-[4-[4-[5-acetyl-3-[7- | δ = 8.71 (s, 2H), 7.66-7.50 (m, 2H), | 975.6 |
| | (difluoromethyl)-6-(1- | 7.45-7.37 (m, 1H), 7.11-7.00 (m, 1H), | |
| | methylpyrazol-4-yl)-3,4- | 6.98 (d, J = 2.4 Hz, 1H), 6.95-6.88 (m, | |
| | dihydro-2H-quinolin-1-yl]-6,7- | 1H), 6.86-6.78 (m, 1H), 6.73-6.36 (m, | |
| | dihydro-4H-pyrazolo[4,3- | 1H), 5.97 (d, J = 8.0 Hz, 1H), 4.31-4.08 | |
| | c]pyridin-1- | (m, 4H), 4.06 (s, 1H), 4.01-3.89 (m, | |
| | yl]cyclohexyl]piperazin-1-yl]- | 8H), 3.80-3.69 (m, 3H), 3.01-2.75 (m, | |
| | N-[3-(3-chloro-4-cyano- | 4H), 2.63 (s, 4H), 2.45-2.16 (m, 6H), | |
| | phenoxy)-2,2,4,4-tetramethyl- | 2.14-2.05 (m, 4H), 1.77 (s, 2H), 1.60 (t, | |
| | cyclobutyl]pyrimidine-5- | J = 12.0 Hz, 2H), 1.25 (d, J = 14.0 Hz, | |
| | carboxamide | 12H). | |

### Example 47: Synthesis of 4-[2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclohexyl]ethynyl]-N-[3-(4-cyano-3-methoxy-phenoxy)-2,2,4,4-tetramethyl-cyclobutyl]benzamide (Compound 292)

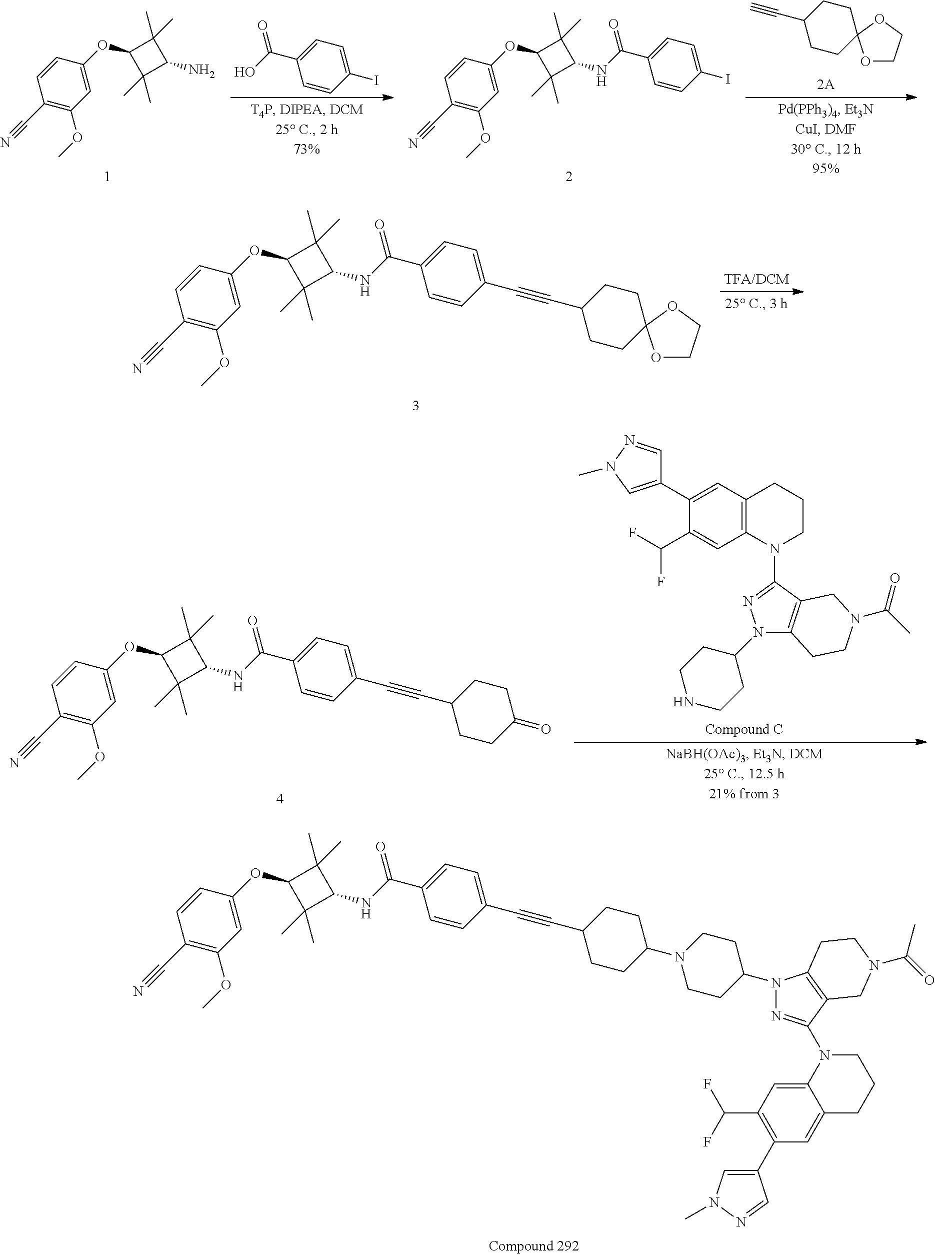

To a solution of 4-(3-amino-2,2,4,4-tetramethyl-cyclobutoxy)-2-methoxy-benzonitrile (1.40 g, 5.10 mmol, 1.0 eq) and 4-iodobenzoic acid (1.52 g, 6.12 mmol, 1.2 eq) in DCM (28 mL) was added DIPEA (3.32 g, 25.7 mmol, 5.0 eq) and T₄P (3.71 g, 10.3 mmol, 2.0 eq). The mixture was stirred at 25° C. for 2 h under N₂ atmosphere. The reaction mixture was quenched by addition water 20 mL, and then diluted with water 40 mL and extracted with ethyl acetate (50 mL×3). The combined organic layers were washed with brine (50 mL), dried over Na₂SO₄, filtered and concentrated under reduced pressure to give a residue. The residue was purified by column chromatography (SiO₂, petroleum ether/ethyl acetate=10/1 to 3/1). The crude product was triturated with petroleum ether/ethyl acetate=3/1 (12 mL) at 25° C. for 60 min. The desired compound N-[3-(4-cyano-3-methoxy-phenoxy)-2,2,4,4-tetramethyl-cyclobutyl]-4-iodo-benzamide (1.89 g, 3.75 mmol, 73% yield) was obtained as white solid.

LC-MS: MS (ESI⁺): t_{R}=0.961 min, m/z=505.1 [M+H⁺]

To a solution of N-[3-(4-cyano-3-methoxy-phenoxy)-2,2,4,4-tetramethyl-cyclobutyl]-4-iodo-benzamide (200 mg, 397 mol, 1.0 eq), 8-ethynyl-1,4-dioxaspiro[4.5]decane (198 mg, 1.19 mmol, 3.0 eq), Et₃N (120 mg, 1.19 mmol, 3.0 eq), CuI (15 mg, 79 μmol, 0.2 eq) and Pd(PPh₃)₄ (92 mg, 79 μmol, 0.2 eq) in DMF (4 mL). The mixture was stirred at 30° C. for 12 h under N₂ atmosphere. The reaction mixture was quenched by addition water 20 mL, and then extracted with ethyl acetate (50 mL×3). The combined organic layers were washed with brine (50 mL), dried over Na₂SO₄, filtered and concentrated under reduced pressure to give a residue. The residue was purified by column chromatography (SiO₂, petroleum ether/ethyl acetate=10/1 to 2/1). The desired compound N-[3-(4-cyano-3-methoxy-phenoxy)-2,2,4,4-tetramethyl-cyclobutyl]-4-[2-(1,4-dioxaspiro[4.5]decan-8-yl)ethynyl]benzamide (204 mg, 376 μmol, 95% yield) was obtained as brown oil.

LC-MS: MS (ESI⁺): t_{R}=1.054 min, m/z=543.4 [M+H⁺]

To a solution of N-[3-(4-cyano-3-methoxy-phenoxy)-2,2,4,4-tetramethyl-cyclobutyl]-4-[2-(1,4-dioxaspiro[4.5]decan-8-yl)ethynyl]benzamide (96 mg, 177 μmol, 1.0 eq) in DCM (2 mL) was added TFA (1.54 g, 13.5 mmol, 76.1 eq). The mixture was stirred at 25° C. for 3 h. The reaction mixture was concentrated under reduced pressure to give a residue. Compound N-[3-(4-cyano-3-methoxy-phenoxy)-2,2,4,4-tetramethyl-cyclobutyl]-4-[2-(4-oxocyclohexyl)ethynyl]benzamide (88 mg) was obtained as a brown oil and directly used in the next step without further purification.

LC-MS: MS (ESI⁺): t_{R}=0.642 min, m/z=499.4 [M+H⁺]

To a solution of N-[3-(4-cyano-3-methoxy-phenoxy)-2,2,4,4-tetramethyl-cyclobutyl]-4-[2-(4-oxocyclohexyl)ethynyl]benzamide (88 mg), 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (90 mg, 177 μmol, 1.0 eq) in DCM (2 mL) was added Et₃N (179 mg, 1.76 mmol, 10.0 eq). The mixture was stirred at 25° C. for 0.5 h. And then NaBH(OAc)₃ (187 mg, 883 μmol, 5.0 eq) was added. The mixture was stirred at 25° C. for 12 h. The reaction mixture was diluted with DCM 150 mL, and then washed by water (30 mL×2). The combined organic layers were dried over Na₂SO₄, filtered and concentrated under reduced pressure to give a residue. The residue was purified by prep-HPLC (FA condition; column: Phenomenex luna C18 150×25 mm×10 um; mobile phase: [water (FA)-ACN]; gradient: 33%-63% B over 10 min). The desired compound 4-[2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclohexyl]ethynyl]-N-[3-(4-cyano-3-methoxy-phenoxy)-2,2,4,4-tetramethyl-cyclobutyl]benzamide (37.71 mg, 37.57 μmol, 21% yield over two steps) was obtained as yellow solid.

¹H NMR (400 MHz, CHLOROFORM-d) δ=9.86-9.62 (m, 1H), 8.67 (s, 2H), 8.07 (s, 2H), 7.89-7.76 (m, 2H), 7.73-7.69 (m, 1H), 7.57 (d, J=8.8 Hz, 1H), 7.54-7.43 (m, 1H), 6.96 (d, J=2.4 Hz, 1H), 6.81-6.78 (m, 1H), 5.94-5.90 (m, 1H), 4.70-4.45 (m, 2H), 4.35-4.23 (m, 1H), 4.22-4.13 (m, 6H), 4.12-4.06 (m, 3H), 4.05-3.99 (m, 2H), 3.83 (t, J=5.6 Hz, 1H), 3.57 (t, J=12.4 Hz, 2H), 2.97-2.81 (m, 2H), 2.51-2.34 (m, 4H), 2.27-2.19 (m, 2H), 2.14-2.01 (m, 4H), 1.98-1.85 (m, 4H), 1.24 (s, 6H), 1.21 (s, 6H)

LC-MS: MS (ESI⁺): t_{R}=2.461 min, m/z=992.6 [M+H⁺]

The compounds below were prepared in a similar manner as described in Example 47.

| | | | LC-MS |
|---|---|---|---|
| Compound | Name | ¹H NMR (CDCl₃) | [M + H⁺] |
| 293 | 2-[2-[4-[4-[5-acetyl-3-[7- | δ = 9.14-9.02 (m, 2H), 8.66 (s, 1H), | 998.4 |
| | (difluoromethyl)-6-(1- | 7.58 (d, J = 8.8 Hz, 1H), 7.53 (d, J = | |
| | methylpyrazol-4-yl)-3,4-dihydro- | 6.0 Hz, 1H), 7.42-7.39 (m, 1H), 7.02 | |
| | 2H-quinolin-1-yl]-6,7-dihydro-4H- | (s, 1H), 6.97 (s, 1H), 6.88-6.78 (m, | |
| | pyrazolo[4,3-c]pyridin-1-yl]-1- | 2H), 6.70-6.26 (m, 2H), 5.49-4.44 | |
| | piperidyl]cyclohexyl]ethynyl]-N- | (m, 1H), 4.39-4.22 (m, 1H), 4.20- | |
| | [3-(3-chloro-4-cyano-phenoxy)- | 4.08 (m, 3H), 4.00-3.93 (m, 4H), 3.91- | |
| | 2,2,4,4-tetramethyl- | 3.88 (m, 1H), 3.76-3.65 (m, 3H), | |
| | cyclobutyl]pyrimidine-5- | 3.21-2.96 (m, 2H), 2.93-2.80 (m, | |
| | carboxamide | 3H), 2.76-2.57 (m, 2H), 2.54-2.45 | |
| | | (m, 3H), 2.21 (s, 2H), 2.18-2.16 (m, | |
| | | 2H), 2.09-2.04 (m, 4H), 2.00 (d, J = | |
| | | 10.0 Hz, 4H), 1.71-1.50 (m, 2H), 1.45- | |
| | | 1.35 (m, 2H), 1.33-1.29 (m, 6H), | |
| | | 1.24 (s, 6H) | |

### Example 48: Synthesis of 4-[2-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutyl]ethynyl]-N-[3-(4-cyano-3-methoxy-phenoxy)-2,2,4,4-tetramethyl-cyclobutyl]benzamide (Compound 294)

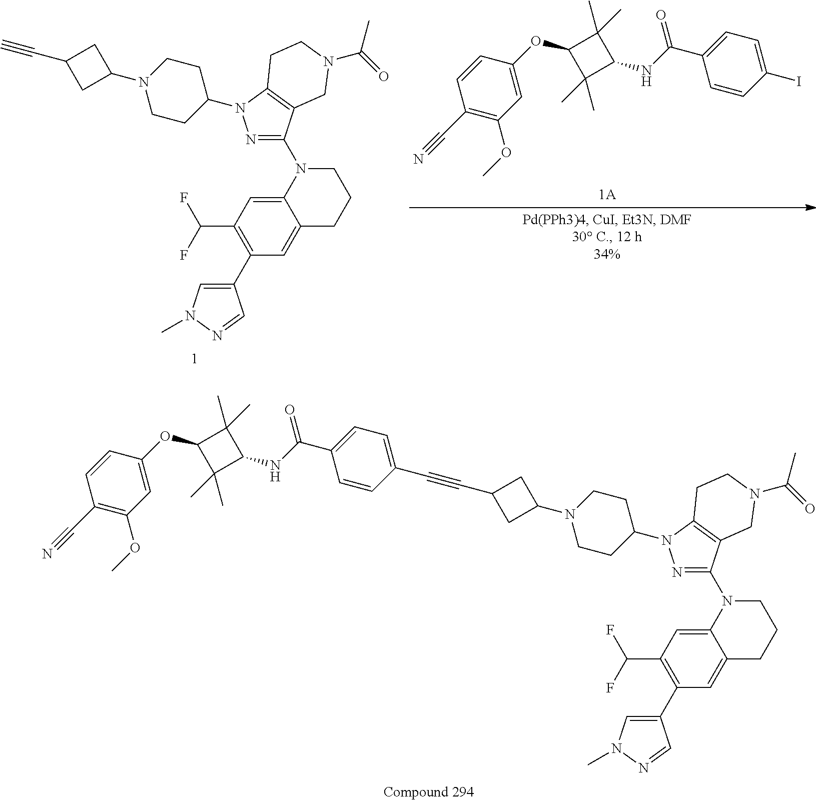 [SMILES: C#CC1CC(N2CCC(n3nc(N4CCCc5cc(-c6cnn(C)c6)c(C(F)F)cc54)c4c3CCN(C(C)=O)C4)CC2)C1 ; COc1cc(O[C@H]2C(C)(C)[C@H](NC(=O)c3ccc(I)cc3)C2(C)C)ccc1C#N ; COc1cc(O[C@H]2C(C)(C)[C@H](NC(=O)c3ccc(C#CC4CC(N5CCC(n6nc(N7CCCc8cc(-c9cnn(C)c9)c(C(F)F)cc87)c7c6CCN(C(C)=O)C7)CC5)C4)cc3)C2(C)C)ccc1C#N]

To a solution of 1-[3-[7-(difluoromethyl)-6-(1-methylpyrazol-4-yl)-3,4-dihydro-2H-quinolin-1-yl]-1-[1-(3-ethynylcyclobutyl)-4-piperidyl]-6,7-dihydro-4H-pyrazolo[4,3-c]pyridin-5-yl]ethanone (80 mg, 136 μmol, 1.0 eq) and N-[3-(4-cyano-3-methoxy-phenoxy)-2,2,4,4-tetramethyl-cyclobutyl]-4-iodo-benzamide (69 mg, 136 μmol, 1.0 eq) in DMF (2 mL) was added Et₃N (41 mg, 408 μmol, 3.0 eq) and CuI (5 mg, 27.2 μmol, 0.2 eq) and Pd(PPh₃)₄ (31 mg, 27.2 μmol, 0.2 eq) at 25° C. The mixture was stirred at 30° C. for 12 h under N₂. The mixture was poured into iced water (20 mL). The aqueous phase was extracted with ethyl acetate (20 mL×3). The combined organic layer was dried with anhydrous Na₂SO₄, concentrated under vacuum. The residue was purified by semi-preparative reverse-phase HPLC (column: Phenomenex Luna C18 150×25 mm×10 um; mobile phase: [water (FA)-ACN]; gradient: 31%-61% B over 10 min). Compound 4-[2-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutyl]ethynyl]-N-[3-(4-cyano-3-methoxy-phenoxy)-2,2,4,4-tetramethyl-cyclobutyl]benzamide (44.60 mg, 46.2 μmol, 34% yield) was obtained as a off-white solid.

¹H NMR (400 MHz, CHLOROFORM-d) δ=7.69 (d, J=8.0 Hz, 2H), 7.53 (d, J=5.6 Hz, 1H), 7.46 (d, J=8.4 Hz, 3H), 7.43-7.37 (m, 1H), 7.08-6.97 (m, 1H), 6.87 (d, J=5.6 Hz, 1H), 6.71-6.35 (m, 3H), 6.20 (d, J=8.0 Hz, 1H), 4.74 (d, J=2.4 Hz, 1H), 4.24 (s, 1H), 4.17-4.09 (m, 2H), 4.06 (s, 1H), 4.00-3.86 (m, 8H), 3.80-3.64 (m, 3H), 3.13-2.99 (m, 2H), 2.97-2.70 (m, 6H), 2.55 (d, J=7.2 Hz, 2H), 2.29-2.19 (m, 3H), 2.18-2.13 (m, 2H), 2.11-2.03 (m, 5H), 2.02-1.95 (m, 2H), 1.27 (s, 6H), 1.25 (s, 6H)

LC-MS: MS (ESI⁺): t_{R}=2.099 min, m/z=964.2 [M+H⁺]

The compounds below were prepared in a similar manner as described in Example 48.

| | | | LC-MS |
|---|---|---|---|
| Compound | Name | ¹H NMR (CDCl₃) | [M + H⁺] |
| 295 | 2-[2-[3-[4-[5-acetyl-3-[7- | δ = 9.03 (s, 2H), 7.63-7.49 (m, 2H), | 970.3 |
| | (difluoromethyl)-6-(1- | 7.41 (d, J = 6.8 Hz, 1H), 7.09-6.94 (m, | |
| | methylpyrazol-4-yl)-3,4-dihydro- | 2H), 6.93-6.76 (m, 2H), 6.76-6.24 | |
| | 2H-quinolin-1-yl]-6,7-dihydro- | (m, 2H), 4.28-4.14 (m, 2H), 4.10 (d, | |
| | 4H-pyrazolo[4,3-c]pyridin-1-yl]- | J = 12.4 Hz, 2H), 3.96 (s, 3H), 3.89 (t, | |
| | 1-piperidyl]cyclobutyl]ethynyl]- | J = 5.6 Hz, 1H), 3.78-3.64 (m, 3H), 3.49 | |
| | N-[3-(3-chloro-4-cyano-phenoxy)- | (s, 4H), 3.19-3.04 (m, 2H), 3.02-2.96 | |
| | 2,2,4,4-tetramethyl- | (m, 1H), 2.91-2.81 (m, 3H), 2.77- | |
| | cyclobutyl]pyrimidine-5- | 2.71 (m, 1H), 2.63-2.53 (m, 2H), 2.41 | |
| | carboxamide | (s, 1H), 2.30-2.23 (m, 2H), 2.16 (s, | |
| | | 2H), 2.11-2.02 (m, 6H), 1.29 (s, 6H), | |
| | | 1.24 (s, 6H) | |

### Example 49: Synthesis of 4-[3-[2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]-5-oxo-7H-pyrrolo[3,4-b]pyridin-6-yl]-2,2,4,4-tetramethyl-cyclobutoxy]-2-chloro-benzonitrile (Compound 296)

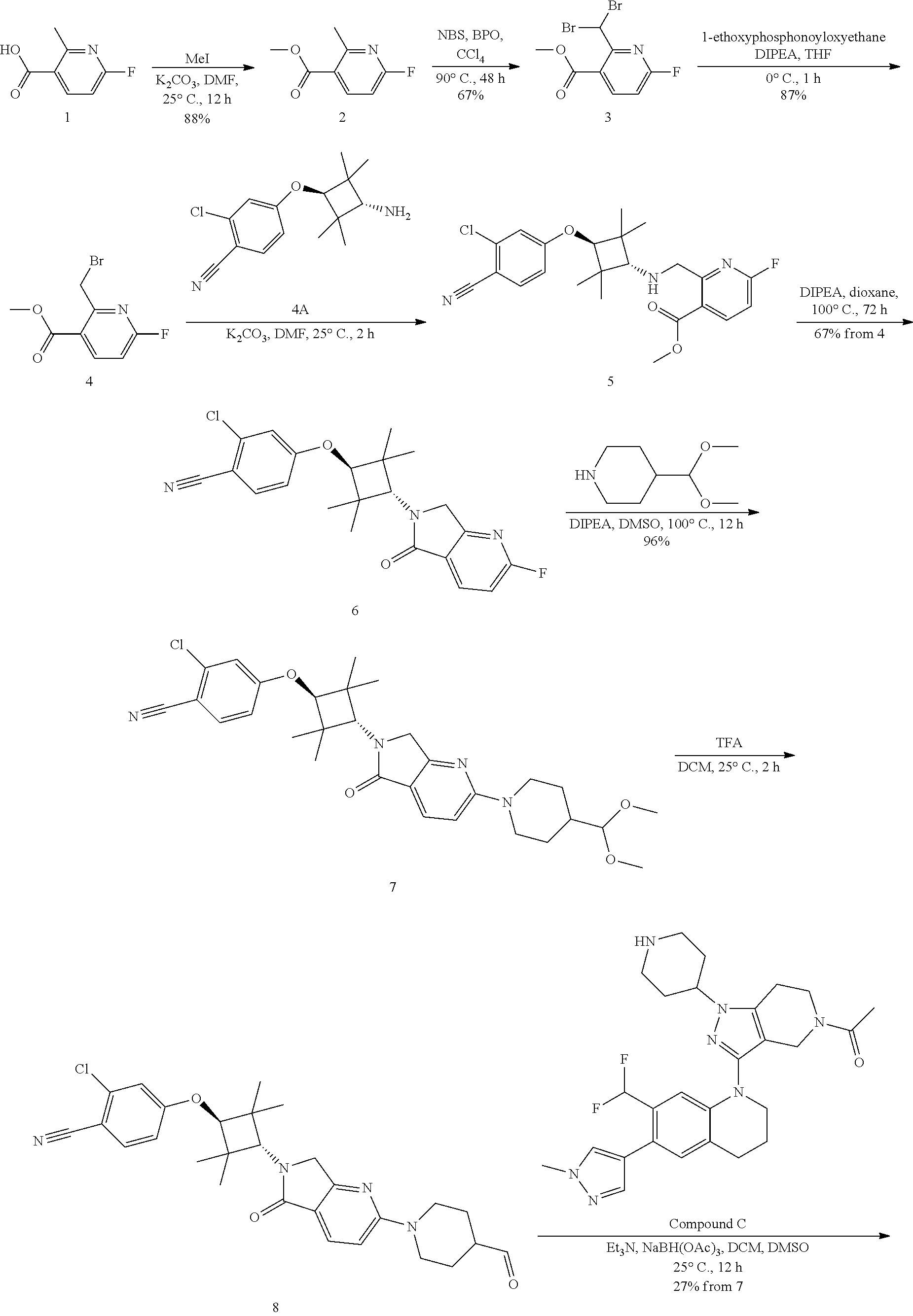

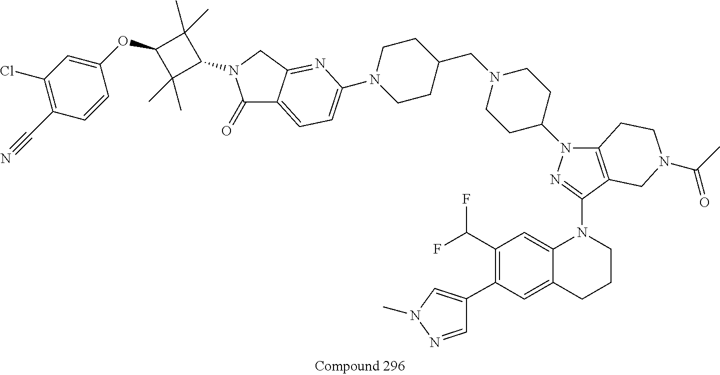 [SMILES: CC(=O)N1CCc2c(c(N3CCCc4cc(-c5cnn(C)c5)c(C(F)F)cc43)nn2C2CCN(CC3CCN(c4ccc5c(n4)CN([C@H]4C(C)(C)[C@H](Oc6ccc(C#N)c(Cl)c6)C4(C)C)C5=O)CC3)CC2)C1]

To a solution of 6-fluoro-2-methyl-pyridine-3-carboxylic acid (2.00 g, 12.89 mmol, 1.0 eq) in DMF (20 mL) was added K₂CO₃ (4.00 g, 28.94 mmol, 2.24 eq) and Mel (2.50 g, 17.61 mmol, 1.10 mL, 1.37 eq). The mixture was stirred at 25° C. for 12 h. The mixture was quenched with water (100 mL) and the aqueous was extracted with ethyl acetate (150 mL×2) and the combined organic layers were washed with brine (120 mL), dried over anhydrous Na₂SO₄, filtered and concentrated under reduced pressure to give a residue. The residue was purified by column chromatography (SiO₂, petroleum ether/ethyl acetate=10/1 to 6/1) to give methyl 6-fluoro-2-methyl-pyridine-3-carboxylate (1.91 g, 11.29 mmol, 88% yield) as a colorless oil.

LC-MS: MS (ESI⁺): t_{R}=0.508 min, m/z=170.1 [M+H⁺]

To a solution of methyl 6-fluoro-2-methyl-pyridine-3-carboxylate (1.91 g, 11.29 mmol, 1.0 eq) in CCl₄ (20 mL) was added BPO (1.4 g, 5.78 mmol, 0.5 eq) and NBS (6.00 g, 33.71 mmol, 3.0 eq). The mixture was stirred at 90° C. for 48 h. The mixture was concentrated under reduced pressure to give a residue. The residue was purified by column chromatography (SiO₂, petroleum ether/ethyl acetate=20/1 to 10/1) to give methyl 2-(dibromomethyl)-6-fluoro-pyridine-3-carboxylate (2.45 g, 7.49 mmol, 67% yield) as a white solid.

LC-MS: MS (ESI⁺: t_{R}=0.574 min, m/z=327.9 [M+H⁺]

To a solution of methyl 2-(dibromomethyl)-6-fluoro-pyridine-3-carboxylate (2.45 g, 7.49 mmol, 1.0 eq) in THF (30 mL) was added DIPEA (4.08 g, 31.58 mol, 5.5 mL, 4.2 eq) and 1-ethoxyphosphonoyloxyethane (3.22 g, 23.29 mmol, 3 mL, 3.1 eq). The mixture was stirred at 0° C. for 1 h. The reaction mixture was quenched by addition water 250 mL at 0° C., and then diluted with ethyl acetate (100 mL) and extracted with ethyl acetate (100 mL×3). The combined organic layers were washed with brine (100 mL×2), dried over Na₂SO₄, filtered and concentrated under reduced pressure to give a residue. The residue was purified by column chromatography (SiO₂, petroleum ether/ethyl acetate=50/1 to 10/1) to give methyl 2-(bromomethyl)-6-fluoro-pyridine-3-carboxylate (1.6 g, 6.45 mmol, 87% yield) as a white solid.

LC-MS: MS (ESI⁺: t_{R}=0.551 min, m/z=248.0 [M+H⁺]

To a solution of 4-(3-amino-2,2,4,4-tetramethyl-cyclobutoxy)-2-chloro-benzonitrile (650 mg, 2.06 mmol, 1.02 eq) and K₂CO₃ (700 mg, 5.06 mmol, 1.0 mL, 2.51 eq) in DMF (2 mL) was added dropwise a solution of methyl 2-(bromomethyl)-6-fluoro-pyridine-3-carboxylate (0.5 g, 2.02 mmol, 1.0 eq) in DMF (1 mL) at 25° C. and stirred at 25° C. for 2 h under N₂. The reaction mixture was quenched by addition water 20 mL at 0° C., and then diluted with ethyl acetate 20 mL and extracted with ethyl acetate (20 mL×3). The combined organic layers were washed with brine (20 mL×2), dried over Na₂SO₄, filtered and concentrated under reduced pressure to give methyl 2-[[[3-(3-chloro-4-cyano-phenoxy)-2,2,4,4-tetramethyl-cyclobutyl]amino]methyl]-6-fluoropyridine-3-carboxylate (0.89 g) as a yellow gum and used into the next step without further purification.

LC-MS: MS (ESI: t_{R}=0.547 min, m/z=446.2 [M+H⁺]

To a solution of methyl 2-[[[3-(3-chloro-4-cyano-phenoxy)-2,2,4,4-tetramethyl-cyclobutyl]amino]methyl]-6-fluoropyridine-3-carboxylate (1.78 g, 3.99 mmol, 1.0 eq) in dioxane (20 mL) was added DIPEA (1.48 g, 11.48 mmol, 2.0 mL, 2.9 eq), the mixture was stirred at 100° C. for 72 h under N₂. The reaction mixture was quenched by addition water 250 mL at 0° C., and then diluted with ethyl acetate 100 mL and extracted with ethyl acetate (100 mL×3). The combined organic layers were washed with brine (100 mL×2), dried over Na₂SO₄, filtered and concentrated under reduced pressure to give a residue. The residue was purified by column chromatography (SiO₂, Dichloromethane/methanol=100/1 to 50/1) to give 2-chloro-4-[3-(2-fluoro-5-oxo-7H-pyrrolo[3,4-b]pyridin-6-yl)-2,2,4,4-tetramethylcyclobutoxy]benzonitrile (1.10 g, 2.66 mmol, 67% yield over two steps) as a white solid.

LC-MS: MS (ESI: t_{R}=0.640 min, m/z=414.2 [M+H⁺]

To a solution of 2-chloro-4-[3-(2-fluoro-5-oxo-7H-pyrrolo[3,4-b]pyridin-6-yl)-2,2,4,4-tetramethyl-cyclobutoxy]benzonitrile (300 mg, 725 mol, 1.0 eq), 4-(dimethoxymethyl)piperidine (127 mg, 798 μmol, 1.1 eq) in DMSO (4 mL) was added DIPEA (297 mg, 2.30 mmol, 0.4 mL, 3.2 eq). The mixture was stirred at 100° C. for 12 h. The reaction was diluted with water (100 mL) and the resulting mixture was extracted with ethyl acetate (100 mL×2). The combined organic layers were dried over Na₂SO₄ and concentrated to give the residue. The residue was purified by column chromatography (SiO₂, petroleum ether/ethyl acetate=1/0 to 1/1) to give 2-chloro-4-[3-[2-[4-(dimethoxymethyl)-1-piperidyl]-5-oxo-7H-pyrrolo[3,4-b]pyridin-6-yl]-2,2,4,4-tetramethyl-cyclobutoxy]benzonitrile (383 mg, 693 μmol, 96% yield) as a white solid.

LC-MS: MS (ESI⁺): t_{R}=0.961 min, m/z=553.3 [M+H⁺]

To a solution of 2-chloro-4-[3-[2-[4-(dimethoxymethyl)-1-piperidyl]-5-oxo-7H-pyrrolo[3,4-b]pyridin-6-yl]-2,2,4,4-tetramethyl-cyclobutoxy]benzonitrile (190 mg, 344 μmol, 1.0 eq) in DCM (2 mL) was added TFA (1 mL). The mixture was stirred at 25° C. for 2 h. The reaction mixture was concentrated to give 2-chloro-4-[3-[2-(4-formyl-1-piperidyl)-5-oxo-7H-pyrrolo[3,4-b]pyridin-6-yl]-2,2,4,4-tetramethyl-cyclobutoxy]benzonitrile (174 mg) as a white solid and used into the next step without further purification.

LC-MS: MS (ESI⁺): t_{R}=0.681 min, m/z=507.2 [M+H⁺]

To a solution of 2-chloro-4-[3-[2-(4-formyl-1-piperidyl)-5-oxo-7H-pyrrolo[3,4-b]pyridin-6-yl]-2,2,4,4-tetramethyl-cyclobutoxy]benzonitrile (174 mg), 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (175 mg, 344 μmol, 1.0 eq) in DCM (1 mL) and DMSO (1 mL) was added Et₃N (182 mg, 1.80 mmol, 0.25 mL, 5.2 eq) at 25° C. over a period of 0.5 h under N₂. Then NaBH(OAc)₃ (220 mg, 1.04 mmol, 3.0 eq) was added. The reaction mixture was stirred at 25° C. for 12 h. The reaction was diluted with water (100 mL) and the resulting mixture was extracted with ethyl acetate (100 mL×2). The combined organic layers were dried over Na₂SO₄ and concentrated to give the residue. The residue was purified by prep-HPLC (column: Phenomenex luna C18 150*25 mm*10 um; mobile phase: [water(FA)-ACN]; gradient: 30%-60% B over 10 min) to give 4-[3-[2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-1-piperidyl]-5-oxo-7H-pyrrolo[3,4-b]pyridin-6-yl]-2,2,4,4-tetramethyl-cyclobutoxy]-2-chloro-benzonitrile (92.46 mg, 91 μmol, 27% yield over two steps) as a white solid.

¹H NMR (400 MHz, DMSO-d₆): δ=7.89 (d, J=8.8 Hz, 1H), 7.75 (s, 1H), 7.69 (d, J=8.8 Hz, 1H), 7.50 (s, 1H), 7.27 (d, J=2.4 Hz, 1H), 7.10 (s, 1H), 7.05 (d, J=2.4, 8.8 Hz, 1H), 6.96-6.63 (m, 3H), 4.59 (s, 2H), 4.50 (s, 1H), 4.43 (d, J=12.0 Hz, 2H), 4.29 (s, 1H), 4.19-4.10 (m, 2H), 4.05-3.99 (m, 1H), 3.86 (s, 3H), 3.73 (d, J=5.2 Hz, 1H), 3.68 (d, J=5.2 Hz, 2H), 3.59 (d, J=5.2 Hz, 2H), 3.17 (s, 1H), 2.98-2.89 (m, 4H), 2.85 (d, J=5.6 Hz, 3H), 2.73 (s, 1H), 2.18 (d, J=6.0 Hz, 2H), 2.08-2.02 (m, 4H), 2.02-1.93 (m, 4H), 1.87-1.78 (m, 4H), 1.37 (s, 6H), 1.18-0.99 (m, 8H).

LC-MS: MS (ESI⁺): t_{R}=1.974 min, m/z=1000.6 [M+H⁺]

SFC: t_{R1}=1.260 min, 99%.

The compounds below were prepared in a similar manner as described in Example 49.

| | | | LC-MS |
|---|---|---|---|
| Compound | Name | ¹H NMR (DMSO-d₆) | [M + H⁺] |
| 297 | 4-[3-[2-[4-[4-[5-acetyl-3-[7- | δ = 7.89 (d, J = 8.8 Hz, 1H), 7.77-7.68 | 986.6 |
| | (difluoromethyl)-6-(1- | (m, 2H), 7.49 (s, 1H), 7.32-7.23 (m, | |
| | methylpyrazol-4-yl)-3,4- | 1H), 7.10 (s, 1H), 7.08-7.03 (m, 1H), | |
| | dihydro-2H-quinolin-1-yl]-6,7- | 6.94-6.63 (m, 3H), 4.60 (s, 2H), 4.52- | |
| | dihydro-4H-pyrazolo[4,3- | 4.45 (m, 3H), 4.29 (s, 1H), 4.19-3.97 | |
| | c]pyridin-1-yl]-1-piperidyl]-1- | (m, 9H), 3.88-3.84 (m, 3H), 3.76-3.65 | |
| | piperidyl]-5-oxo-7H- | (m, 3H), 3.60-3.55 (m, 3H), 3.02-2.88 | |
| | pyrrolo[3,4-b]pyridin-6-yl]- | (m, 5H), 2.84 (d, J = 4.8 Hz, 3H), 2.71- | |
| | 2,2,4,4-tetramethyl- | 2.60 (m, 2H), 2.34-2.27 (m, 2H), 2.09- | |
| | cyclobutoxy]-2-chloro- | 2.05 (m, 4H), 2.02-1.93 (m, 5H), 1.88- | |
| | benzonitrile | 1.80 (m, 4H), 1.52-1.40 (m, 3H), 1.37 | |
| | | (s, 5H), 1.30-1.16 (m, 2H), 1. 13 (s, 5H), | |
| | | 1.09-1.02 (m, 1H), 0.91-0.78 (m, 1H) | |
| 298 | 4-[3-[2-[9-[5-acetyl-3-[7- | δ = 7.89 (d, J = 8.8 Hz, 1H), 7.75 (s, 1H), | 971.6 |
| | (difluoromethyl)-6-(1- | 7.69 (d, J = 8.8 Hz, 1H), 7.50 (s, 1H), | |
| | methylpyrazol-4-yl)-3,4- | 7.27 (d, J = 2.4 Hz, 1H), 7.10 (br s, 1H), | |
| | dihydro-2H-quinolin-1-yl]-6,7- | 7.07-7.02 (m, 1H), 6.96-6.62 (m, 3H), | |
| | dihydro-4H-pyrazolo[4,3-c] | 4.59 (s, 2H), 4.50 (s, 1H), 4.29 (s, 1H), | |
| | pyridin-1-yl]-3-azaspiro [5.5] | 4.22-4.12 (m, 2H), 4.10-4.00 (m, 1H), | |
| | undecan-3-yl]-5-oxo-7H- | 3.87 (s, 3H), 3.74 (br d, J = 5.2 Hz, 1H), | |
| | pyrrolo[3,4-b] pyridin-6-yl]- | 3.67 (br s, 5H), 3.62-3.55 (m, 2H), 2.91- | |
| | 2,2,4,4-tetramethyl- | 2.71 (m, 4H), 2.13-1.97 (m, 7H), 1.85 | |
| | cyclobutoxy]-2-chloro- | (br d, J = 10.4 Hz, 2H), 1.74 (br d, J = | |
| | benzonitrile | 8.8 Hz, 2H), 1.62 (br s, 2H), 1.43-1.28 | |
| | | (m, 9H), 1.25-1.19 (m, 1H), 1.13 (s, | |
| | | 6H) | |
| 299 | 4-[3-[2-[4-[4-[5-acetyl-3-[7- | δ = 7.89 (d, J = 8.8 Hz, 1H), 7.77-7.73 | 1014.6 |
| | (difluoromethyl)-6-(1- | (m, 1H), 7.71 (d, J = 8.8 Hz, 1H), 7.50 (s, | |
| | methylpyrazol-4-yl)-3,4- | 1H), 7.27 (d, J = 2.4 Hz, 1H), 7.10 (s, | |
| | dihydro-2H-quinolin-1-yl]-6,7- | 1H), 7.05 (dd, J = 2.4, 8.8 Hz, 1H), 6.93- | |
| | dihydro-4H-pyrazolo[4,3- | 6.66 (m, 3H), 4.60 (s, 2H), 4.51-4.41 | |
| | c]pyridin-1-yl]piperidine-1- | (m, 4H), 4.39-4.33 (m, 1H), 4.29 (s, | |
| | carbonyl]-1-piperidyl]-5-oxo- | 1H), 4.16 (br d, J = 18.4 Hz, 3H), 3.86 (s, | |
| | 7H-pyrrolo[3,4-b]pyridin-6-yl]- | 3H), 3.78-3.67 (m, 2H), 3.61-3.55 (m, | |
| | 2,2,4,4-tetramethyl- | 2H), 3.22-3.14 (m, 1H), 3.06 (br t, J = | |
| | cyclobutoxy]-2-chloro- | 11.6 Hz, 3H), 2.88 (br d, J = 6.0 Hz, 1H), | |
| | benzonitrile | 2.84 (br s, 2H), 2.78-2.64 (m, 2H), 2.08 | |
| | | (s, 2H), 2.01-1.88 (m, 6H), 1.71 (br d, | |
| | | J = 11.2 Hz, 3H), 1.61-1.49 (m, 2H), 1.37 | |
| | | (s, 6H), 1.13 (s, 6H) | |

### Example 50: Synthesis of 4-[4-[4-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]-1-piperidyl]-3, 3-difluoro-1l-piperidyl]-N-[3-(4-cyano-3-methoxy-phenoxy)-2,2,4,4-tetramethyl-cyclobutyl]benzamide (Compound 302)

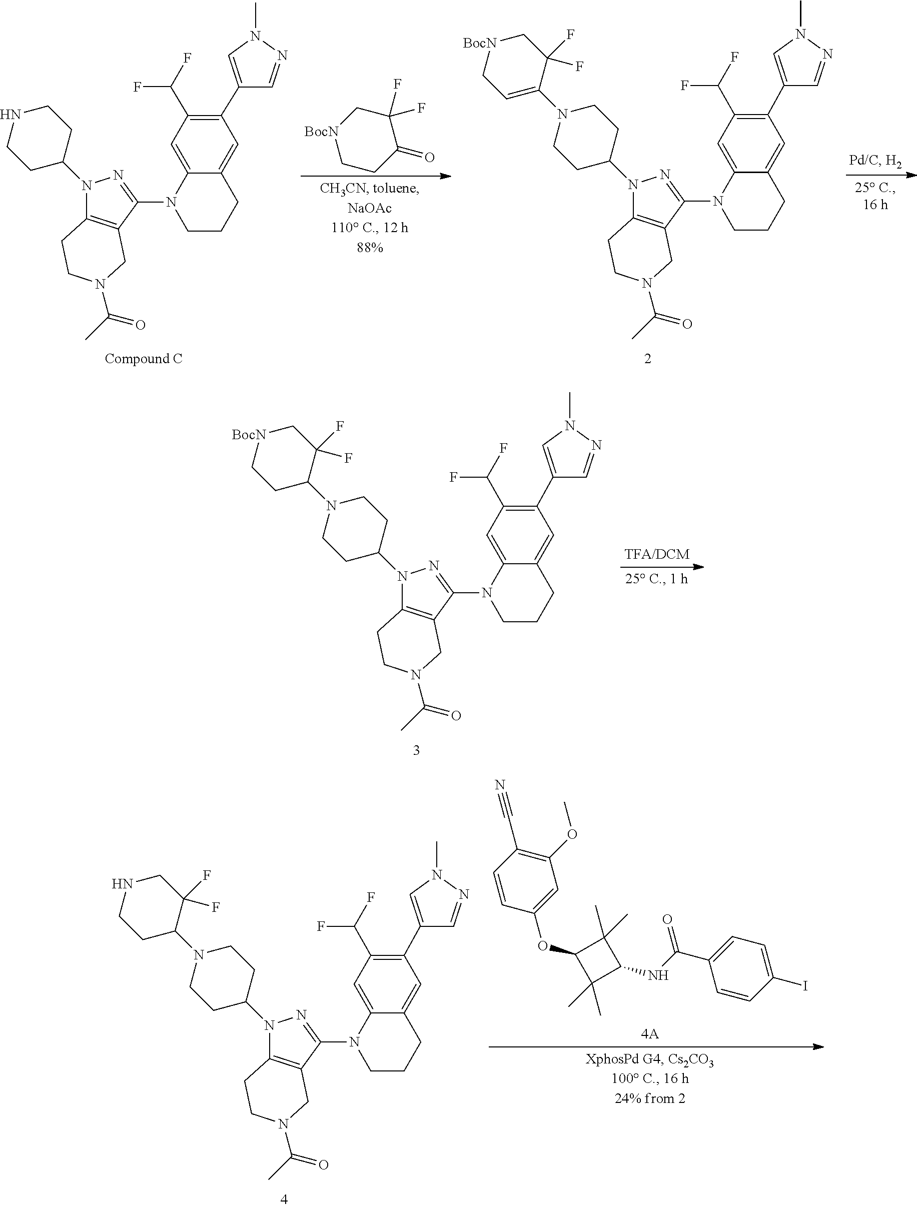

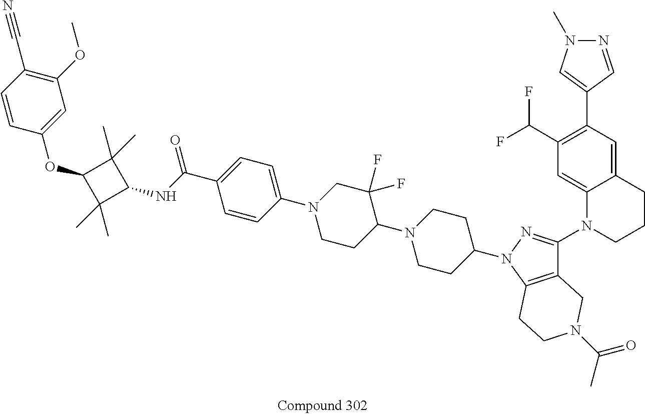 [SMILES: COc1cc(O[C@H]2C(C)(C)[C@H](NC(=O)c3ccc(N4CCC(N5CCC(n6nc(N7CCCc8cc(-c9cnn(C)c9)c(C(F)F)cc87)c7c6CCN(C(C)=O)C7)CC5)C(F)(F)C4)cc3)C2(C)C)ccc1C#N]

A mixture of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (300 mg, 549 μmol, 1.0 eq, HCl salt), tert-butyl 3,3-difluoro-4-oxo-piperidine-1-carboxylate (388 mg, 1.65 mmol, 3.0 eq), NaOAc (180 mg, 2.20 mmol, 4.0 eq), HOAc (99 mg, 1.65 mmol, 94 μL, 3.0 eq) and 4A MS (2 g) in CH₃CN (12 mL) and ToI. (12 mL) was degassed and purged with N₂ for 3 times, and then the mixture was stirred at 110° C. for 12 h under N₂ atmosphere. The mixture was filtered and the filter cake was washed with EA (100 mL). The solution was washed with saturated NaHCO₃ (20 mL), brine (20 mL), dried, filtered, and concentrated to give a residue. The residue was purified by prep-TLC (EA) to give the desired product tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-3,3-difluoro-2,6-dihydropyridine-1-carboxylate (350 mg, 482 μmol, 88% yield) as a yellow solid.

LC-MS: MS (ESI⁺) t_{R}=0.611 min, m/z=727.3 [M+H⁺]

Pd/C (500 mg) was added into a 100 mL single-necked round bottom flask under N₂, and then THF (20 mL) was added at 25° C. under N₂. After addition, tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-3,3-difluoro-2,6-dihydropyridine-1-carboxylate (350 mg, 482 μmol, 1.0 eq) was added under N₂. The suspension was degassed under vacuum and purged with H₂ several times. The mixture was stirred under H₂ (15 psi) at 25° C. for 16 h. The reaction mixture was filtered and the filter was concentrated to afford product. The product was used into the next without further purification. Compound tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-3,3-difluoro-piperidine-1-carboxylate (180 mg) was obtained as a yellow solid.

LC-MS: MS (ESI⁺): t_{R}=0.507 min, m/z=729.4 [M+H⁺]

To a solution of tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-3,3-difluoro-piperidine-1-carboxylate (180 mg) in DCM (5 mL) was added TFA (1 mL). The mixture was stirred at 25° C. for 1 h. Saturated NaHCO₃ aqueous solution was added to reaction mixture until pH=7-8. Then the mixture was extracted with DCM (20 mL×3). The combined organic layers were washed with brine (20 mL), dried over anhydrous sodium sulfate, filtered, and concentrated under reduce pressure to give product. Compound 1-[3-[7-(difluoromethyl)-6-(1-methylpyrazol-4-yl)-3,4-dihydro-2H-quinolin-1-yl]-1-[1-(3,3-difluoro-4-piperidyl)-4-piperidyl]-6,7-dihydro-4H-pyrazolo[4,3-c]pyridin-5-yl]ethanone (155 mg) was obtained as a yellow solid.

LC-MS: MS (ESI⁺): t_{R}=0.496 min, m/z=629.3 [M+H⁺]

To a solution of 1-[3-[7-(difluoromethyl)-6-(1-methylpyrazol-4-yl)-3,4-dihydro-2H-quinolin-1-yl]-1-[1-(3,3-difluoro-4-piperidyl)-4-piperidyl]-6,7-dihydro-4H-pyrazolo[4,3-c]pyridin-5-yl]ethanone (75 mg) and N-[3-(4-cyano-3-methoxy-phenoxy)-2,2,4,4-tetramethyl-cyclobutyl]-4-iodo-benzamide (72 mg, 143 μmol, 1.2 eq) in dioxane (2 mL) was added Xphos Pd G4 (21 mg, 24 μmol, 0.2 eq) and Cs₂CO₃ (117 mg, 358 μmol, 3.0 eq). The mixture was stirred at 100° C. for 16 h under N₂ atmosphere. The mixture was concentrated to give a residue. The residue was purified by prep-HPLC (column: Waters xbridge 150*25 mm 10 um; mobile phase: [water (ammonia hydroxide v/v)-ACN]; gradient: 46%-76% B over 9 min). Compound 4-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-3,3-difluoro-1-piperidyl]-N-[3-(4-cyano-3-methoxy-phenoxy)-2,2,4,4-tetramethyl-cyclobutyl]benzamide (57.16 mg, 55.81 μmol, 24% yield over three steps) was obtained as an off-white solid.

¹H NMR (400 MHz, CD₃OD) δ=7.76-7.74 (d, J=9.2 Hz, 2H), 7.64 (s, 1H), 7.53-7.51 (d, J=8.8 Hz, 2H), 7.09 (d, J=8.4 Hz, 1H), 7.03-7.01 (d, J=9.2 Hz, 2H), 6.72 (s, 3H), 6.57-6.55 (m, 1H), 6.55-6.42 (m, 2H), 4.26 (s, 1H), 4.21 (m, 2H), 4.13-4.04 (m, 4H), 3.93 (m, 6H), 3.81 (m, 1H), 3.75 (m, 1H), 3.68-3.67 (m, 2H), 3.25-2.93 (m, 5H), 2.91-2.82 (m, 2H), 2.18-1.93 (m, 11H), 1.41-1.15 (m, 12H).

LC-MS: MS (ESI⁺): t_{R}=2.028 min, m/z=1005.4 [M+H⁺]

### Example 51: Synthesis of 4-[2-[[5-[1-(5-acetyl-1-tetrahydropyran-4-yl-6,7-dihydro-4H-pyrazolo[4,3-c]pyridin-3-yl)-7-(difluoromethyl)-3,4-dihydro-2H-quinolin-6-yl]-2-pyridyl]oxy]-7-azaspiro[3.5]nonan-7-yl]-N-[3-(4-cyano-3-methoxy-phenoxy)-2,2,4, 4-tetramethyl-cyclobutyl]benzamide (Compound 306)

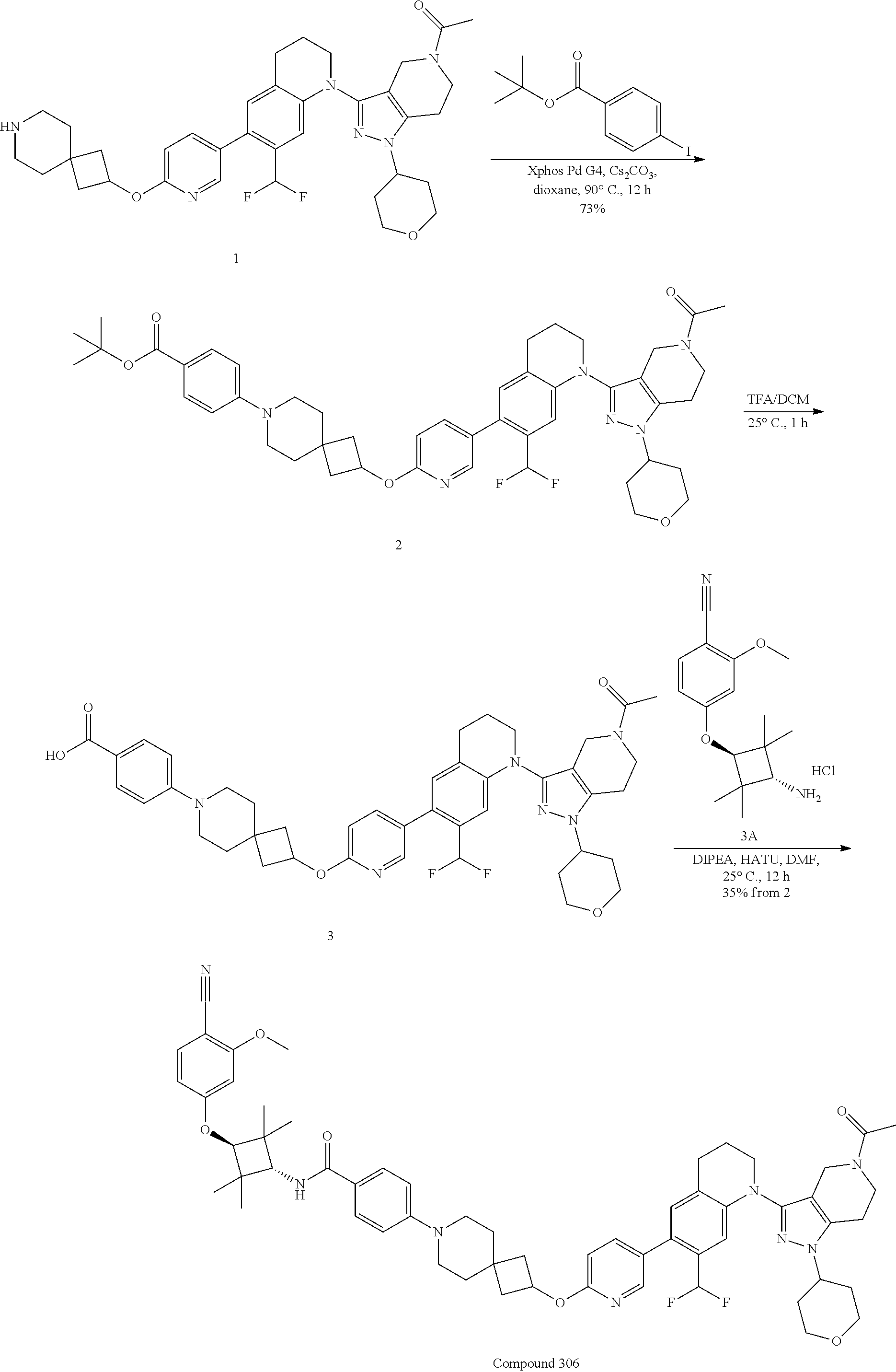

To a mixture of 1-[3-[6-[6-(7-azaspiro[3.5]nonan-2-yloxy)-3-pyridyl]-7-(difluoromethyl)-3,4-dihydro-2H-quinolin-1-yl]-1-tetrahydropyran-4-yl-6,7-dihydro-4H-pyrazolo[4,3-c]pyridin-5-yl]ethanone (150 mg, 231 μmol, 1.0 eq) and tert-butyl 4-iodobenzoate (105 mg, 347 μmol, 1.5 eq) in dioxane (3 mL) was added Cs₂CO₃ (226 mg, 695 μmol, 3.0 eq) and Xphos Pd G4 (19 mg, 23 μmol, 0.1 eq) under N₂. The mixture was stirred at 90° C. for 12 h. The mixture was filtered and concentrated to give a residue. The residue was purified by prep-TLC (SiO₂, DCM/MeOH=10/1). Compound tert-butyl 4-[2-[[5-[1-(5-acetyl-1-tetrahydropyran-4-yl-6,7-dihydro-4H-pyrazolo[4,3-c]pyridin-3-yl)-7-(difluoromethyl)-3,4-dihydro-2H-quinolin-6-yl]-2-pyridyl]oxy]-7-azaspiro[3.5]nonan-7-yl]benzoate (142 mg, 170 μmol, 73% yield) was obtained as a white solid.

LC-MS: MS (ESI⁺): t_{R}=0.998 min, m/z=823.3 [M+H⁺]

To a mixture of tert-butyl 4-[2-[[5-[1-(5-acetyl-1-tetrahydropyran-4-yl-6,7-dihydro-4H-pyrazolo[4,3-c]pyridin-3-yl)-7-(difluoromethyl)-3,4-dihydro-2H-quinolin-6-yl]-2-pyridyl]oxy]-7-azaspiro[3.5]nonan-7-yl]benzoate (142 mg, 172 μmol, 1.0 eq) in DCM (3 mL) was added TFA (1.54 g, 13.4 mmol, 1 mL, 78.0 eq). The mixture was stirred at 25° C. for 1 h. The mixture was concentrated to give a crude product. Compound 4-[2-[[5-[1-(5-acetyl-1-tetrahydropyran-4-yl-6,7-dihydro-4H-pyrazolo[4,3-c]pyridin-3-yl)-7-(difluoromethyl)-3,4-dihydro-2H-quinolin-6-yl]-2-pyridyl]oxy]-7-azaspiro[3.5]nonan-7-yl]benzoic acid (132 mg) was obtained as a yellow solid and directly used into the next step without further purification.

LC-MS: MS (ESI⁺): t_{R}=0.872 min, m/z=767.3 [M+H⁺]

To a mixture of 4-[2-[[5-[1-(5-acetyl-1-tetrahydropyran-4-yl-6,7-dihydro-4H-pyrazolo[4,3-c]pyridin-3-yl)-7-(difluoromethyl)-3,4-dihydro-2H-quinolin-6-yl]-2-pyridyl]oxy]-7-azaspiro[3.5]nonan-7-yl]benzoic acid (132 mg) and 4-(3-amino-2,2,4,4-tetramethyl-cyclobutoxy)-2-methoxy-benzonitrile (53 mg, 172 μmol, 1.0 eq) in DMF (2 mL) was added DIPEA (111 mg, 860 μmol, 149 μL, 5.0 eq) and HATU (78 mg, 206 μmol, 1.2 eq). The mixture was stirred at 25° C. for 12 h. The mixture was filtered and concentrated to give a residue. The residue was purified by prep-HPLC (column: Phenomenex luna C18 150*25 mm*10 um; mobile phase: [water (FA)-ACN]; gradient: 65%-95% B over 10 min). Compound 4-[2-[[5-[1-(5-acetyl-1-tetrahydropyran-4-yl-6,7-dihydro-4H-pyrazolo[4,3-c]pyridin-3-yl)-7-(difluoromethyl)-3,4-dihydro-2H-quinolin-6-yl]-2-pyridyl]oxy]-7-azaspiro[3.5]nonan-7-yl]-N-[3-(4-cyano-3-methoxy-phenoxy)-2,2,4,4-tetramethyl-cyclobutyl]benzamide (62.61 mg, 60.41 μmol, 35% yield over two steps) was obtained as a yellow solid.

¹H NMR (400 MHz, DMSO-d₆) δ=8.10-8.00 (m, 1H), 7.81-7.70 (m, 2H), 7.68-7.57 (m, 2H), 7.56-7.43 (m, 1H), 7.08-6.93 (m, 3H), 6.90-6.82 (m, 2H), 6.81-6.50 (m, 3H), 5.32-5.15 (m, 1H), 4.37-4.25 (m, 2H), 4.23-4.13 (m, 2H), 4.09-4.02 (m, 1H), 3.99-3.88 (m, 5H), 3.80-3.68 (m, 2H), 3.65-3.57 (m, 2H), 3.51-3.43 (m, 2H), 3.31-3.27 (m, 2H), 3.25-3.17 (m, 2H), 2.91-2.74 (m, 4H), 2.48-2.39 (m, 2H), 2.08 (s, 2H), 2.03-1.94 (m, 5H), 1.93-1.79 (m, 4H), 1.76-1.63 (m, 4H), 1.22 (s, 6H), 1.14 (s, 6H).

LC-MS: MS (ESI⁺): t_{R}=2.917 min, m/z=1023.6 [M+H⁺]

The compounds below were prepared in a similar manner as described in Example 51.

| | | | LC-MS |
|---|---|---|---|
| Compound | Name | ¹H NMR (CDCl₃) | [M + H⁺] |
| 307 | 4-[2-[4-[1-(5-acetyl-1- | δ = 7.69 (d, J = 8.8 Hz, 2 H) 7.46 (d, | 1022.4 |
| | tetrahydropyran-4-yl-6,7- | J = 8.8 Hz, 1 H) 7.22 (d, J = 8.4 Hz, 2 H) | |
| | dihydro-4H-pyrazolo[4,3- | 7.02-6.91 (m, 3 H) 6.90-6.80 (m, 3 H) | |
| | c]pyridin-3-yl)-7- | 6.61-6.27 (m, 3 H) 6.12 (d, J = 8.4 Hz, 1 | |
| | (difluoromethyl)-3,4-dihydro- | H) 4.83-4.69 (m, 1 H) 4.28 (s, 1 H) 4.21- | |
| | 2H-quinolin-6-yl] phenoxy]-7- | 4.09 (m, 5 H) 4.05 (s, 1 H) 3.92 (s, 4 H) | |
| | azaspiro[3.5]nonan-7-yl]-N-[3- | 3.80-3.68 (m, 3 H) 3.53 (m, 2 H) 3.37- | |
| | (4-cyano-3-methoxy-phenoxy)- | 3.22 (m, 4 H) 2.95-2.80 (m, 3 H) 2.76 | |
| | 2,2,4,4-tetramethyl- | (m, 1 H) 2.55-2.44 (m, 2 H) 2.39-2.24 | |
| | cyclobutyl]benzamide | (m, 2 H) 2.17 (s, 1 H) 2.14-1.99 (m, 6 | |
| | | H) 1.88 (m, 2 H) 1.83-1.77 (m, 4 H) | |
| | | 1.25 (d, J = 10.0 Hz, 12 H) | |

### Example 52: Synthesis of 2-[4-[7-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]spiro[3.5]nonan-2-yl]piperazin-1-yl]-N-[3-(3-chloro-4-cyano-phenoxy)-2,2,4,4-tetramethyl-cyclobutyl]pyrimidine-5-carboxamide (Compound

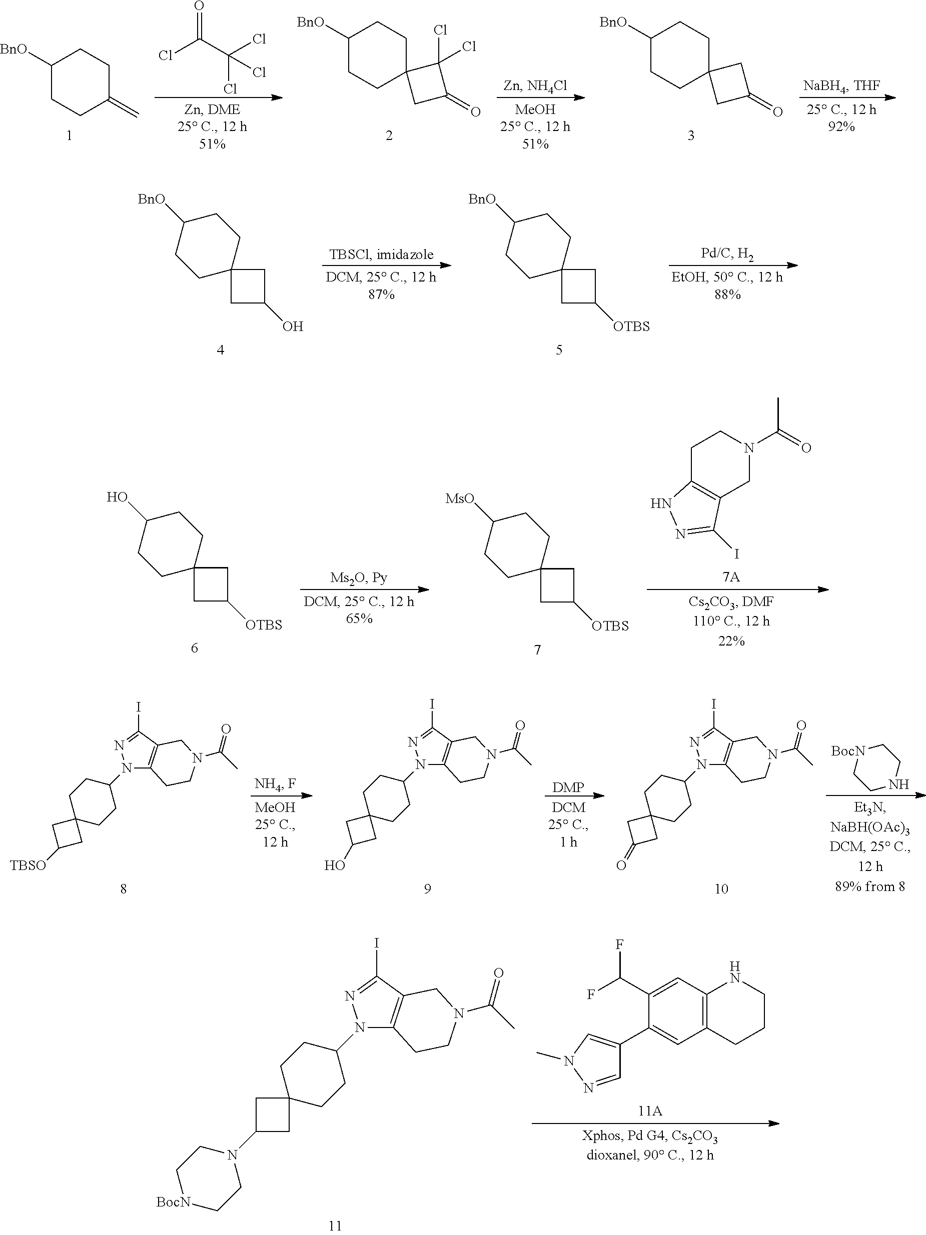

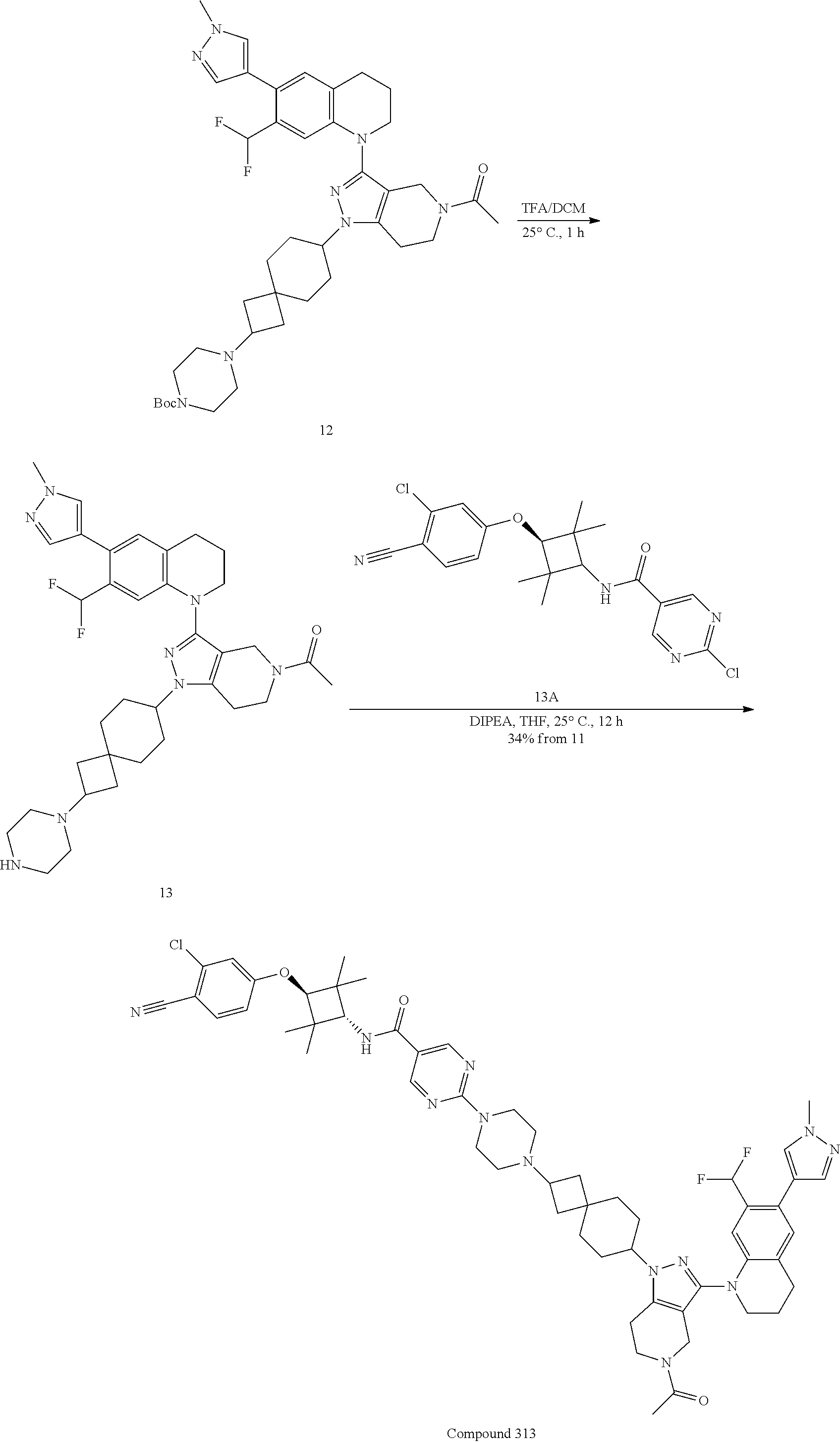

Synthesis of 1 was reported in Bioorganic and Medicinal Chemistry Letters, 2019, vol. 29, #16, p. 2100-2106.

Synthesis of 7A was reported in Journal of Medicinal Chemistry, 2024, vol. 67, #7, p. 5275-5304.

Synthesis of 11A was reported in Journal of Medicinal Chemistry, 2017, vol. 60, #22, p. 9162-9183

Synthesis of 13A was reported in Journal of Medicinal Chemistry, 2023, vol. 66, #7, p. 4784-4801.

To a solution of (4-methylenecyclohexoxy)methylbenzene (23.00 g, 113.7 mmol, 1.0 eq), Zn (29.93 g, 457.7 mmol, 4.0 eq) in dioxane (230 mL) was added the solution of 1,2-dimethoxyethane (20.49 g, 227.4 mmol, 23.6 mL, 2.0 eq) and 2,2,2-trichloroacetyl chloride (31.01 g, 170.6 mmol, 190 mL, 1.5 eq) in dioxane (100 mL) dropwise under N₂. The mixture was stirred at 25° C. for 12 h under N₂ atmosphere. The reaction mixture was adjusted with saturated aqueous NaHCO₃ (150 mL) solution to pH=7-8 and then extracted with EA (200 mL×3). The combined organic layers were dried, filtered, and concentrated under reduced pressure to give a residue. The residue was purified by column chromatography (SiO₂, petroleum ether/ethyl acetate=1/0 to 5/1) to give the product 7-benzyloxy-3,3-dichloro-spiro[3.5]nonan-2-one (18.00 g, 57.47 mmol, 51% yield) as a light yellow gum.

LC-MS: MS (ESI⁺) t_{R}=0.662 min, m/z=335.1 [M+Na⁺]

A mixture of 7-benzyloxy-3,3-dichloro-spiro[3.5]nonan-2-one (18.00 g, 57.47 mmol, 1.0 eq), Zn (18.58 g, 284.1 mmol, 4.9 eq), NH₄Cl (18.44 g, 344.8 mmol, 6.0 eq) in MeOH (180 mL) was degassed and purged with N₂ for 3 times, and then the mixture was stirred at 25° C. for 12 h under N₂ atmosphere. The reaction mixture was adjusted with saturated aqueous NaHCO₃ (150 mL) solution to pH=7-8 and then extracted with EA (200 mL×3). The combined organic layers were dried, filtered, and concentrated under reduced pressure to give a residue. The residue was purified by column chromatography (SiO₂, petroleum ether/ethyl acetate=1/0 to 3/1) to give the product 7-benzyloxyspiro[3.5]nonan-2-one (7.20 g, 29.47 mmol, 51% yield) as a colorless oil.

¹H NMR (400 MHz, CDCl₃) δ=7.36-7.30 (m, 4H), 7.29-7.28 (m, 1H), 4.58 (s, 2H), 3.50-3.48 (m, 1H), 2.81-2.78 (d, J=2.4 Hz, 4H), 1.89-1.87 (m, 4H), 1.63-1.55 (m, 4H).

To a solution of 7-benzyloxyspiro[3.5]nonan-2-one (7.20 g, 29.47 mmol, 1.0 eq) in THF (75 mL) was added NaBH₄ (2.23 g, 58.94 mmol, 2.0 eq) at 0° C. under N₂. The mixture was stirred at 25° C. for 12 h under N₂. The mixture was quenched by addition sat. NH₄Cl (50 mL), diluted with water (50 mL), extracted with EtOAc (100 mL×3). The combined organic layers were dried over Na₂SO₄, filtered, and concentrated under reduced pressure to give a residue. The residue was purified by column chromatography (SiO₂, petroleum ether/ethyl acetate=50/1 to 5/1) to give the product 7-benzyloxyspiro[3.5]nonan-2-ol (6.70 g, 27.20 mmol, 92% yield) as a colorless oil.

¹H NMR (400 MHz, CD₃OD) δ=7.35-7.32 (m, 4H), 7.28 (m, 1H), 4.54 (s, 2H), 4.23-4.18 (m, 1H), 3.39 (m, 1H), 2.26 (m, 1H), 2.14 (m, 1H), 1.83-1.81 (m, 2H), 1.71-1.62 (m, 4H), 1.40-1.28 (m, 4H).

A mixture of 7-benzyloxyspiro[3.5]nonan-2-ol (6.70 g, 27.20 mmol, 1.0 eq), imidazole (5.55 g, 81.59 mmol, 3.0 eq), and tert-butyl-chloro-dimethyl-silane (6.15 g, 40.80 mmol, 5.0 mL, 1.5 eq) in DCM (60 mL) was degassed and purged with N₂ for 3 times, and then the mixture was stirred at 25° C. for 12 h under N₂ atmosphere. The mixture was diluted with water (30 mL), extracted with DCM (100 mL×3). The combined organic layers were dried over Na₂SO₄, filtered, and concentrated under reduced pressure to give a residue. The residue was purified by column chromatography (SiO₂, petroleum ether/ethyl acetate=50/1 to 5/1) to give the product (7-benzyloxyspiro[3.5]nonan-2-yl)oxy-tert-butyl-dimethyl-silane (8.50 g, 23.57 mmol, 87% yield) as a light yellow oil.

¹H NMR (400 MHz, CD₃OD) δ=7.32-7.29 (m, 4H), 7.27-7.25 (m, 1H), 4.51 (s, 2H), 4.29-4.26 (m, 1H), 3.38-3.35 (m, 1H), 2.23 (m, 1H), 2.11 (m, 1H), 1.82 (m, 2H), 1.68-1.61 (m, 4H), 1.39-1.32 (m, 4H), 0.88 (s, 9H), 0.05 (s, 6H).

To a solution of (7-benzyloxyspiro[3.5]nonan-2-yl)oxy-tert-butyl-dimethyl-silane (8.50 g, 23.57 mmol, 1.0 eq) in EtOH (100 mL) was added Pd/C (2.5 g) under N₂ atmosphere. The suspension was degassed and purged with H₂ for three times. The mixture was stirred under H₂ at 50° C. for 12 h. The reaction mixture was filtered and concentrated under reduced pressure to give a residue. The crude product was used into next step without further purification. Compound 2-[tert-butyl(dimethyl)silyl]oxyspiro[3.5]nonan-7-ol (5.60 g, 20.70 mmol, 88% yield) as yellow gum.

¹H NMR (400 MHz, CD₃OD) δ=4.25-4.22 (m, 1H), 3.47-3.45 (m, 1H), 2.20 (m, 1H), 2.05 (m, 1H), 1.68-1.56 (m, 6H), 1.31-1.28 (m, 4H), 0.86 (s, 9H), 0.01 (s, 6H).

To a solution of 2-[tert-butyl(dimethyl)silyl]oxyspiro[3.5]nonan-7-ol (5.60 g, 20.70 mmol, 1.0 eq) in DCM (60 mL) was added Py (8.19 g, 103.52 mmol, 8.4 mL, 5.0 eq) and the solution of methylsulfonyl methanesulfonate (10.82 g, 62.11 mmol, 3.0 eq) in DCM (15 mL) at 0° C. under N₂. The mixture was stirred at 25° C. for 12 h under N₂. The reaction mixture was diluted with DCM (150 mL), washed with saturated NaCl (50 mL×3), dried over dried over Na₂SO₄, filtered, and concentrated under reduced pressure to give a residue. The residue was purified by column chromatography (SiO₂, petroleum ether/ethyl acetate=1/0 to 5/1) to give the product [2-[tert-butyl(dimethyl)silyl]oxyspiro[3.5]nonan-7-yl]methanesulfonate (4.70 g, 13.48 mmol, 65% yield) as a light yellow solid.

¹H NMR (400 MHz, CDCl₃) δ=4.71-4.66 (m, 1H), 4.25-4.20 (m, 1H), 3.02 (s, 3H), 2.22-2.21 (m, 1H), 2.12 (m, 1H), 1.87 (m, 2H), 1.72-1.64 (m, 6H), 1.46-1.41 (m, 2H), 0.92 (s, 9H), 0.05 (s, 6H).

A mixture of 1-(3-iodo-1,4,6,7-tetrahydropyrazolo[4,3-c]pyridin-5-yl)ethanone (3.92 g, 13.48 mmol, 1.0 eq), [2-[tert-butyl(dimethyl)silyl]oxyspiro[3.5]nonan-7-yl]methanesulfonate (4.70 g, 13.48 mmol, 1 eq), and Cs₂CO₃ (13.18 g, 40.45 mmol, 3.0 eq) in DMF (40 mL) was degassed and purged with N₂ for 3 times, and then the mixture was stirred at 110° C. for 12 h under N₂ atmosphere. The reaction mixture was diluted with water (20 mL), extracted with EA (100 mL×3). The combined organic layers were washed with brine (30 mL×3), dried over Na₂SO₄, filtered, and concentrated under reduced pressure to give a residue. The residue was purified by prep-HPLC (column: Phenomenex luna C18 (250*70 mm, 10 um); mobile phase: [water(FA)-ACN]; gradient: 60%-90% B over 30 min) to give the product 1-[1-[2-[tert-butyl(dimethyl)silyl]oxyspiro[3.5]nonan-7-yl]-3-iodo-6,7-dihydro-4H-pyrazolo[4,3-c]pyridin-5-yl]ethanone (1.60 g, 2.94 mmol, 22% yield) as a white solid.

LC-MS: MS (ESI⁺: t_{R}=0.739 min, m/z=544.2 [M+H⁺]

To a solution of 1-[1-[2-[tert-butyl(dimethyl)silyl]oxyspiro[3.5]nonan-7-yl]-3-iodo-6,7-dihydro-4H-pyrazolo[4,3-c]pyridin-5-yl]ethanone (300 mg, 552 μmol, 1.0 eq) in MeOH (10 mL) was added NH₄F (204 mg, 5.52 mmol, 10.0 eq). The mixture was stirred at 25° C. for 12 h. The mixture was diluted with water (15 mL), extracted with DCM (70 mL×3). The combined organic layers were dried over Na₂SO₄, filtered, and concentrated under reduce pressure to give the product 1-[1-(2-hydroxyspiro[3.5]nonan-7-yl)-3-iodo-6,7-dihydro-4H-pyrazolo[4,3-c]pyridin-5-yl]ethanone (240 mg) as a white solid. The crude product was used into next step without further purification.

LC-MS: MS (ESI⁺: t_{R}=0.465 min, m/z=430.1 [M+H⁺]

To a solution of 1-[1-(2-hydroxyspiro[3.5]nonan-7-yl)-3-iodo-6,7-dihydro-4H-pyrazolo[4,3-c]pyridin-5-yl]ethanone (240 mg) in DCM (5 mL) was added DMP (474 mg, 1.12 mmol, 346 μL, 2.0 eq) at 0° C. The mixture was stirred at 25° C. for 1 h under N₂. The mixture was quenched by addition sat. NaHCO₃ (15 mL). The solution was extracted with DCM (70 mL×3). The combined organic layers were dried over Na₂SO₄, filtered, and concentrated under reduce pressure to give the product 7-(5-acetyl-3-iodo-6,7-dihydro-4H-pyrazolo[4,3-c]pyridin-1-yl)spiro[3.5]nonan-2-one (238 mg) as yellow gum. The crude product was used into next step without further purification.

LC-MS: MS (ESI: t_{R}=0.485 min, m/z=428.1 [M+H⁺]

To a solution of 7-(5-acetyl-3-iodo-6,7-dihydro-4H-pyrazolo[4,3-c]pyridin-1-yl)spiro[3.5]nonan-2-one (238 mg), tert-butyl piperazine-1-carboxylate (124 mg, 668 μmol, 1.2 eq) in DCM (5 mL) was added Et₃N (113 mg, 1.11 mmol, 155 μL, 2.0 eq) and NaBH(OAc)₃ (590 mg, 2.79 mmol, 5.0 eq). The mixture was stirred at 25° C. for 12 h under N₂. The mixture was quenched by addition sat. NaHCO₃ (15 mL). The solution was extracted with DCM (50 mL×3). The combined organic layers were dried over Na₂SO₄, filtered, and concentrated under reduce pressure to give a residue. The residue was purified by column chromatography (SiO₂, petroleum ether/ethyl acetate=10/1 to dichloromethane/methanol=10/1) to give the product tert-butyl 4-[7-(5-acetyl-3-iodo-6,7-dihydro-4H-pyrazolo[4,3-c]pyridin-1-yl)spiro[3.5]nonan-2-yl]piperazine-1-carboxylate (295 mg, 494 μmol, 89% yield over three steps) as a white solid.

LC-MS: MS (ESI: t_{R}=0.468 min, m/z=598.3 [M+H⁺]

To a solution of tert-butyl 4-[7-(5-acetyl-3-iodo-6,7-dihydro-4H-pyrazolo[4,3-c]pyridin-1-yl)spiro[3.5]nonan-2-yl]piperazine-1-carboxylate (155 mg, 259 μmol, 1.0 eq), 7-(difluoromethyl)-6-(1-methylpyrazol-4-yl)-1,2,3,4-tetrahydroquinoline (68 mg, 259 μmol, 1.0 eq) in dioxane (2 mL) was added Xphos Pd G4 (22 mg, 25.94 μmol, 0.1 eq) and Cs₂CO₃ (254 mg, 778.20 μmol, 3.0 eq). The mixture was stirred at 90° C. for 12 h under N₂. The reaction mixture was filtered and concentrated under reduce pressure to give a residue. The residue was purified by prep-TLC (dichloromethane:methanol=10:1) to give the product tert-butyl 4-[7-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]spiro[3.5]nonan-2-yl]piperazine-1-carboxylate (50 mg) as a light yellow solid.

LC-MS: MS (ESI⁺): t_{R}=0.504 min, m/z=733.5 [M+H⁺]

To a solution of tert-butyl 4-[7-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]spiro[3.5]nonan-2-yl]piperazine-1-carboxylate (50 mg) in DCM (1 mL) was added TFA (0.5 mL). The mixture was stirred at 25° C. for 1 h. The mixture was quenched by addition sat. NaHCO₃ (15 mL). The solution was extracted with DCM (20 mL×3). The combined organic layers were dried over Na₂SO₄, filtered, and concentrated under reduce pressure to give the product 1-[3-[7-(difluoromethyl)-6-(1-methylpyrazol-4-yl)-3,4-dihydro-2H-quinolin-1-yl]-1-(2-piperazin-1-ylspiro[3.5]nonan-7-yl)-6,7-dihydro-4H-pyrazolo[4,3-c]pyridin-5-yl]ethanone (43 mg) as a yellow solid. The crude product was used into next step without further purification.

LC-MS: MS (ESI⁺): t_{R}=0.481 min, m/z=633.4 [M+H⁺]

To a solution of 1-[3-[7-(difluoromethyl)-6-(1-methylpyrazol-4-yl)-3,4-dihydro-2H-quinolin-1-yl]-1-(2-piperazin-1-ylspiro[3.5]nonan-7-yl)-6,7-dihydro-4H-pyrazolo[4,3-c]pyridin-5-yl]ethanone (43 mg), 2-chloro-N-[3-(3-chloro-4-cyano-phenoxy)-2,2,4,4-tetramethyl-cyclobutyl]pyrimidine-5-carboxamide (28 mg, 68 μmol, 1.0 eq) in THF (1 mL) was added DIPEA (26 mg, 204 μmol, 35 μL, 3.0 eq). The mixture was stirred at 25° C. for 12 h under N₂. The reaction mixture was concentrated under reduced pressure to give a residue. The residue was purified by prep-HPLC (column: Phenomenex luna C18 150*25 mm*10 um; mobile phase: [water(FA)-ACN]; gradient: 26%-56% B over 10 min) to give the product 2-[4-[7-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]spiro[3.5]nonan-2-yl]piperazin-1-yl]-N-[3-(3-chloro-4-cyano-phenoxy)-2,2,4,4-tetramethyl-cyclobutyl]pyrimidine-5-carboxamide (24.52 mg, 23.31 μmol, 34% yield over three steps) as a white solid.

¹H NMR (400 MHz, CD₃OD) δ=8.75 (s, 2H), 7.73-7.71 (d, J=8.8 Hz, 1H), 7.64 (s, 1H), 7.51 (s, 1H), 7.13-7.09 (m, 2H), 6.99-6.97 (m, 1H), 6.73-6.42 (m, 2H), 4.59 (s, 2H), 4.27 (m, 1H), 4.23-4.21 (m, 2H), 4.13 (m, 1H), 3.96 (m, 3H), 3.93 (m, 3H), 3.92 (m, 1H), 3.81 (m, 1H), 3.65 (m, 2H), 2.90-2.89 (m, 3H), 2.81 (m, 2H), 2.49 (m, 4H), 2.25 (m, 1H), 2.18 (m, 2H), 2.08 (m, 2H), 2.03 (m, 3H), 1.81 (m, 3H), 1.78 (m, 3H), 1.61 (m, 3H), 1.28 (s, 6H), 1.21 (s, 6H).

LC-MS: MS (ESI⁺): t_{R}=3.132 min, m/z=1015.7 [M+H⁺]

### Example 53: Synthesis of 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]methyl]piperazin-1-yl]-N—[(1R)-3-(4-cyano-3-methoxy-phenoxy)-1-isopropyl-2,2-dimethyl-propyl]benzamide (Compound 317)

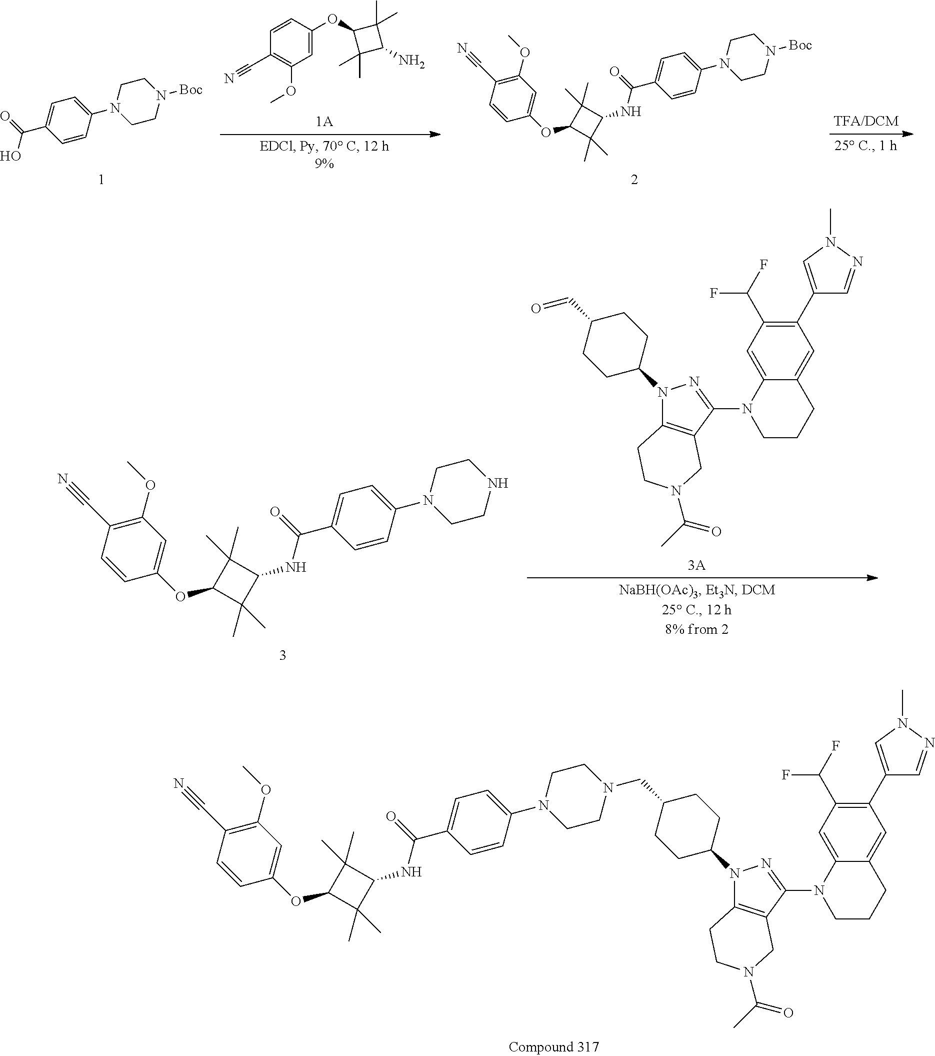

To a solution of 4-(4-tert-butoxycarbonylpiperazin-1-yl)benzoic acid (150 mg, 490 μmol, 1.0 eq) in pyridine (5 mL) was added 4-(3-amino-2,2,4,4-tetramethyl-cyclobutoxy)-2-methoxy-benzonitrile (161 mg, 588 μmol, 1.2 eq) and EDCI (94 mg, 490 μmol, 1.0 eq). The mixture was stirred at 70° C. for 12 h. The reaction mixture was diluted with DCM 100 mL, and then washed by water (20 mL×3). The combined organic layers were dried over Na₂SO₄, filtered and concentrated under reduced pressure to give a residue. The residue was purified by column chromatography (SiO₂, petroleum ether/ethyl acetate=1/1). The desired compound tert-butyl 4-[4-[[3-(4-cyano-3-methoxy-phenoxy)-2,2,4,4-tetramethyl-cyclobutyl]carbamoyl]phenyl]piperazine-1-carboxylate (68 mg, 44 μmol, 9% yield) was obtained as white solid.

LC-MS: MS (ESI⁺): t_{R}=0.597 min, m/z=563.4 [M+H⁺]

To a solution of tert-butyl 4-[4-[[3-(4-cyano-3-methoxy-phenoxy)-2,2,4,4-tetramethyl-cyclobutyl]carbamoyl]phenyl]piperazine-1-carboxylate (68 mg, 121 μmol, 1.0 eq) in DCM (1 mL) was added TFA (768 mg, 6.73 mmol, 0.5 mL, 55.7 eq). The mixture was stirred at 25° C. for 1 h. The reaction mixture was filtered and concentrated under reduced pressure to give a residue. Compound N-[3-(4-cyano-3-methoxy-phenoxy)-2,2,4,4-tetramethyl-cyclobutyl]-4-piperazin-1-yl-benzamide (68 mg) was obtained as yellow oil and directly used in the next step without further purification.

LC-MS: MS (ESI⁺): t_{R}=0.524 min, m/z=463.2 [M+H⁺]

To a solution of N-[3-(4-cyano-3-methoxy-phenoxy)-2,2,4,4-tetramethyl-cyclobutyl]-4-piperazin-1-yl-benzamide (68 mg) and 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexanecarbaldehyde (63 mg, 118 μmol, 1.0 eq) in DCM (5 mL) was added Et₃N (119 mg, 1.18 mmol, 164 μL, 10.0 eq) and NaBH(OAc)₃ (125 mg, 590 μmol, 5.0 eq). The mixture was stirred at 25° C. for 12 h. The reaction mixture was diluted with DCM 50 mL, and then washed by water (10 mL×2). The combined organic layers were dried over Na₂SO₄, filtered and concentrated under reduced pressure to give a residue. The residue was purified by column chromatography (SiO₂, dichloromethane/methanol=10/1). The residue was purified by prep-HPLC (FA column: Unisil 3-100 C18 Ultra 150×50 mm×3 um; mobile phase: [water(FA)-ACN]; gradient: 30%-60% B over 10 min). The desired compound 4-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]methyl]piperazin-1-yl]-N-[(1R)-3-(4-cyano-3-methoxy-phenoxy)-1-isopropyl-2,2-dimethyl-propyl]benzamide (9 mg, 8.86 mol, 80 yield over two steps) was obtained as off-white solid.

¹H NMR (400 MHz, CHLOROFORM-d) δ=7.70 (d, J=8.8 Hz, 2H), 7.54 (d, J=5.6 Hz, 1H), 7.46 (d, J=8.8 Hz, 1H), 7.43-7.38 (m, 1H), 7.05 (s, 1H), 6.96-6.85 (m, 3H), 6.71-6.34 (m, 3H), 6.12 (d, J=8.0 Hz, 1H), 4.26 (s, 1H), 4.17-4.12 (m, 2H), 4.05 (s, 1H), 4.00-3.83 (m, 9H), 3.80-3.61 (m, 3H), 3.45-3.17 (m, 4H), 2.94-2.84 (m, 2H), 2.81 (t, J=5.6 Hz, 1H), 2.75 (t, J=5.6 Hz, 1H), 2.66-2.47 (m, 4H), 2.31-2.20 (m, 2H), 2.30-2.16 (m, 1H), 2.12-2.04 (m, 5H), 2.01 (s, 5H), 1.25 (d, J=10.0 Hz, 12H), 1.14-1.06 (m, 2H)

LC-MS: MS (ESI⁺): t_{R}=3.043 min m/z=983.8 [M+H⁺]

The compounds below were prepared in a similar manner as described in Example 53.

| | | | LC-MS |
|---|---|---|---|
| Compound | Name | ¹H NMR (CDCl₃) | [M + H⁺] |
| 318 | 2-[4-[[4-[5-acetyl-3-[7- | δ = 8.72 (s, 2 H) 7.59 (d, J = 8.8 Hz, 1 | 989.7 |
| | (difluoromethyl)-6-(1- | H) 7.55 (d, J = 5.2 Hz, 1 H) 7.42 (d, | |
| | methylpyrazol-4-yl)-3,4-dihydro- | J = 6.0 Hz, 1 H) 7.06 (s, 1 H) 7.02- | |
| | 2H-quinolin-1-yl]-6,7-dihydro-4H- | 6.96 (m, 1 H) 6.88 (d, J = 3.6 Hz, 1 H) | |
| | pyrazolo[4,3-c] pyridin-1- | 6.82 (m, 1 H) 6.72-6.35 (m, 1 H) 5.94 | |
| | yl]cyclohexyl]methyl]piperazin-1- | (d, J = 8.0 Hz, 1 H) 4.28 (s, 1 H) 4.17- | |
| | yl]-N-[3-(3-chloro-4-cyano- | 4.12 (m, 2 H) 4.06 (s, 1 H) 4.00-3.89 | |
| | phenoxy)-2,2,4,4-tetramethyl- | (m, 9 H) 3.79-3.68 (m, 3 H) 2.92- | |
| | cyclobutyl]pyrimidine-5- | 2.74 (m, 4 H) 2.49 (m, 4 H) 2.24 (m, 2 | |
| | carboxamide | H) 2.18 (s, 1 H) 2.14-1.99 (m, 10 H) | |
| | | 1.27 (s, 6 H) 1.23 (s, 6 H) 1.11 (m, 2 | |
| | | H) | |
| 319 | 4-[4-[[4-[5-acetyl-3-[7- | (CD₃OD) δ = 7.75-7.73 (d, J = 8.8 | 985.7 |
| | (difluoromethyl)-6-(1- | Hz, 2H), 7.64 (s, 1H), 7.53-7.51 (d, | |
| | methylpyrazol-4-yl)-3,4-dihydro- | J = 8.4 Hz, 2H), 7.09 (m, 1H), 7.00- | |
| | 2H-quinolin-1-yl]-6,7-dihydro-4H- | 6.98 (d, J = 9.2 Hz, 2H), 6.75 (s, 1H), | |
| | pyrazolo[4,3-c]pyridin-1- | 6.63-6.43 (m, 3H), 4.26-4.23 (m, | |
| | yl]cyclohexyl]methyl]piperazin-1- | 3H), 4.13 (m, 2H), 3.93 (m, 6H), 3.89- | |
| | yl]-N-[3-(4-cyano-3-methoxy- | 3.81 (m, 2H), 3.68-3.67 (m, 2H), | |
| | phenoxy)-2,2,4,4-tetramethyl- | 3.35 (m, 2H), 2.91 (m, 3H), 2.85 (m, | |
| | cyclobutyl]benzamide | 1H), 2.64-2.63 (m, 4H), 2.55-2.53 | |
| | | (m, 2H), 2.19 (m, 2H), 2.10-2.04 (m, | |
| | | 6H), 1.91 (m, 2H), 1.75 (m, 4H), 1.41- | |
| | | 1.15 (m, 14H) | |
| 320 | 2-[4-[[4-[5-acetyl-3-[7- | (CD₃OD) δ = 8.74 (s, 2H), 7.73-7.71 | 989.7 |
| | (difluoromethyl)-6-(1- | (d, J = 8.4 Hz, 1H), 7.64 (s, 1H), 7.51 | |
| | methylpyrazol-4-yl)-3,4-dihydro- | (s, 1H), 7.13-7.09 (m, 2H), 6.99- | |
| | 2H-quinolin-1-yl]-6,7-dihydro-4H- | 6.97 (d, J = 8.8 Hz, 1H), 6.75 (s, 1H), | |
| | pyrazolo[4,3-c]pyridin-1- | 6.75-6.42 (m, 1H), 4.27-4.23 (m, | |
| | yl]cyclohexyl]methyl]piperazin-1- | 3H), 4.13 (m, 2H), 3.93 (m, 7H), 3.89 | |
| | yl]-N-[3-(3-chloro-4-cyano- | (m, 1H), 3.68-3.67 (m, 1H), 3.65 (m, | |
| | phenoxy)-2,2,4,4-tetramethyl- | 2H), 2.92 (m, 3H), 2.85 (m, 1H), 2.55- | |
| | cyclobutyl]pyrimidine-5- | 2.52 (m, 6H), 2.18 (s, 2H), 2.10- | |
| | carboxamide | 2.04 (m, 3H), 1.93 (m, 2H), 1.75-1.72 | |
| | | (m, 4H), 1.28 (s, 6H), 1.21 (s, 6H) | |

### Example 54: Synthesis of 2-[4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexoxy]-1-piperidyl]-N-[3-(3-chloro-4-cyano-phenoxy)-2,2,4,4-tetramethyl-cyclobutyl]pyrimidine-5-carboxamide (Compound 339)

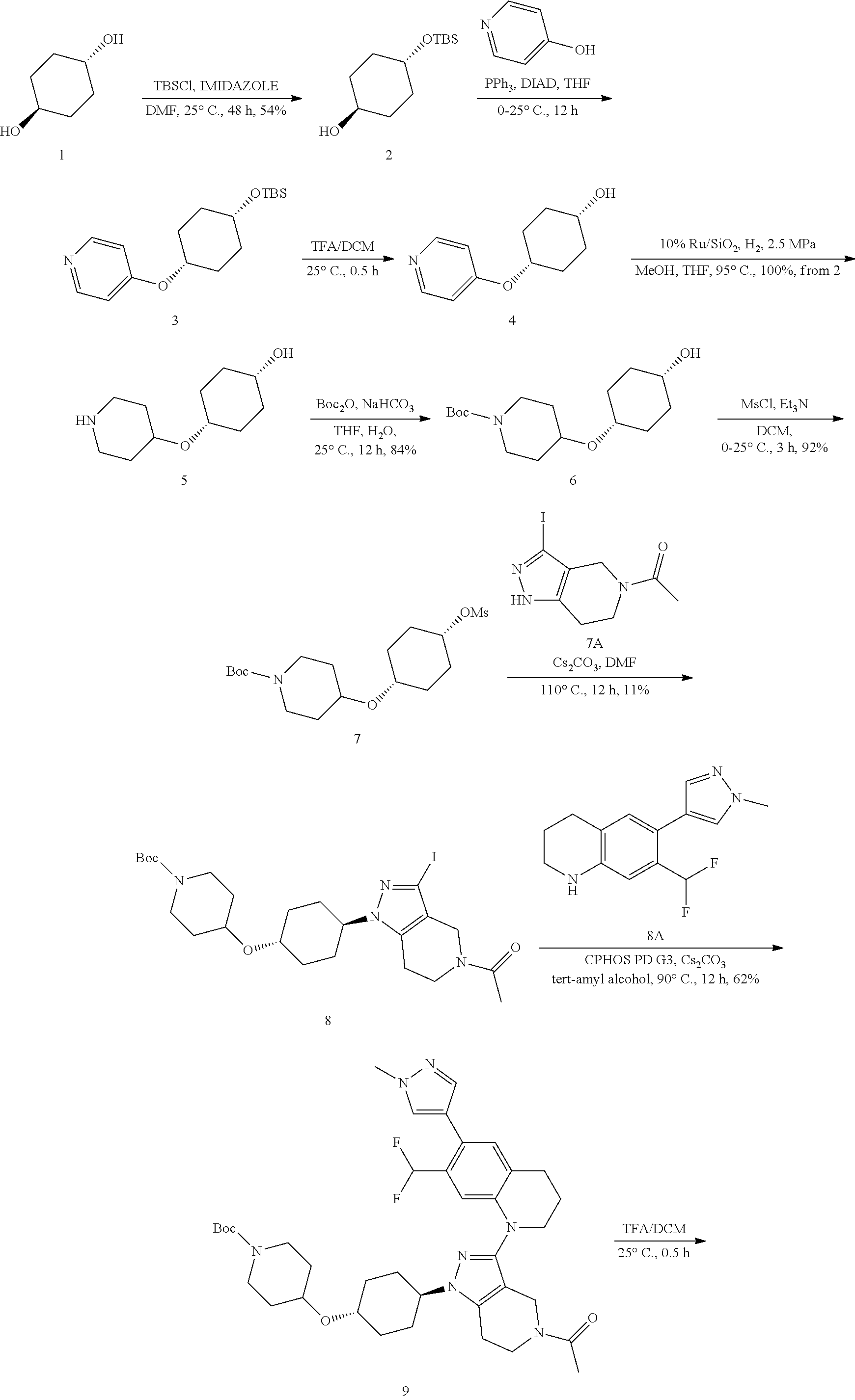

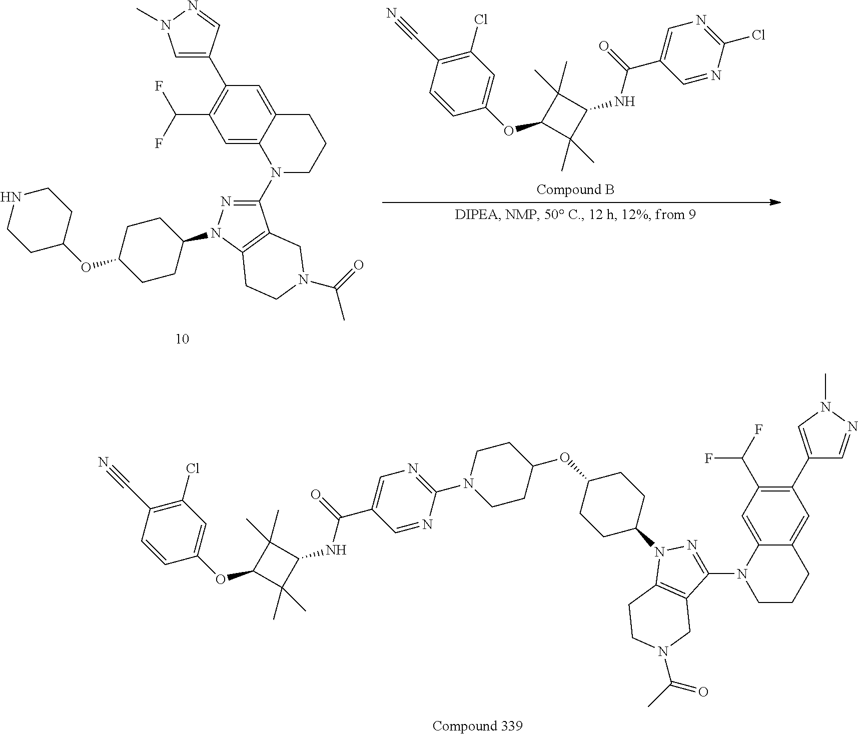 [SMILES: CC(=O)N1CCc2c(c(N3CCCc4cc(-c5cnn(C)c5)c(C(F)F)cc43)nn2[C@H]2CC[C@H](OC3CCNCC3)CC2)C1 ; CC1(C)[C@H](NC(=O)c2cnc(Cl)nc2)C(C)(C)[C@H]1Oc1ccc(C#N)c(Cl)c1 ; CC(=O)N1CCc2c(c(N3CCCc4cc(-c5cnn(C)c5)c(C(F)F)cc43)nn2[C@H]2CC[C@H](OC3CCN(c4ncc(C(=O)N[C@H]5C(C)(C)[C@H](Oc6ccc(C#N)c(Cl)c6)C5(C)C)cn4)CC3)CC2)C1]

To a solution of cyclohexane-1,4-diol (10.00 g, 86.09 mmol, 1.0 eq), IMIDAZOLE (8.80 g, 129.26 mmol, 1.5 eq) in DMF (100 mL) was added TBSCl (14.27 g, 94.66 mmol, 11.65 mL, 1.1 eq). The mixture was stirred at 25° C. for 48 h. The mixture was poured into water (300 mL). The aqueous phase was extracted with ethyl acetate (100 mL×2). The combined organic phase was washed with brine (200 mL×3), dried with anhydrous sodium sulfate, filtered and concentrated in vacuum. The residue was purified by silica gel chromatography (0%-15% ethyl acetate in petroleum ether). Compound 4-[tert-butyl(dimethyl)silyl]oxycyclohexanol (10.80 g, 46.87 mmol, 54% yield) was obtained as a colourless oil.

¹H NMR (400 MHz, CDCl₃) δ=3.75-3.53 (m, 2H), 2.01-1.90 (m, 2H), 1.89-1.78 (m, 2H), 1.43-1.28 (m, 4H), 0.88 (s, 9H), 0.05 (s, 6H)

To a solution of 4-[tert-butyl(dimethyl)silyl]oxycyclohexanol (15.67 g, 68.01 mmol, 1.0 eq), pyridin-4-ol (6.47 g, 68.01 mmol, 1.0 eq) and PPh₃ (17.84 g, 68.01 mmol, 1.0 eq) in THF (160 mL) was added DIAD (16.50 g, 81.61 mmol, 15.82 mL, 1.2 eq) at 0° C. The mixture was stirred at 25° C. for 12 h. The mixture was poured into water (50 mL). The aqueous phase was extracted with ethyl acetate (50 mL×2). The combined organic phase was washed with brine (100 mL×3), dried with anhydrous sodium sulfate, filtered and concentrated in vacuum. The residue was purified by silica gel chromatography (5%-25% ethyl acetate in petroleum ether). Compound tert-butyl-dimethyl-[4-(4-pyridyloxy)cyclohexoxy]silane (25.57 g) was obtained as a colourless oil.

¹H NMR (400 MHz, CDCl₃) δ=8.41-8.37 (m, 2H), 6.81-6.76 (m, 2H), 4.44-4.30 (m, 1H), 3.91-3.77 (m, 1H), 2.02-1.94 (m, 2H), 1.80-1.69 (m, 4H), 1.64-1.53 (m, 2H), 0.90 (s, 9H), 0.06 (s, 6H)

LC-MS: MS (ESI⁺): t_{R}=0.504 min, m/z=308.1 [M+H⁺]

To a solution of tert-butyl-dimethyl-[4-(4-pyridyloxy)cyclohexoxy]silane (22.57 g, 73.40 mmol, 1.0 eq) in dichloromethane (90 mL) was added trifluoroacetic acid (138.15 g, 1.21 mol, 90 mL, 16.51 eq). The mixture was stirred at 25° C. for 0.5 h. The mixture was poured into water (30 mL). The aqueous mixture was adjusted to pH=7 using NaHCO₃. The aqueous phase was extracted with ethyl acetate (30 mL×2). The combined organic phase was washed with brine (70 mL×3), dried with anhydrous sodium sulfate, filtered and concentrated in vacuum. The residue was purified by silica gel chromatography (10%-100% ethyl acetate in petroleum ether, and 0%-20% methanol in dichloromethane). Compound 4-(4-pyridyloxy)cyclohexanol (6.07 g, 31.41 mmol, 42% yield) was obtained as a white solid.

¹H NMR (400 MHz, CDCl₃) δ=8.25-8.48 (m, 2H), 6.70-6.89 (m, 2H), 4.48 (tt, J=5.8, 2.8 Hz, 1H), 3.82 (quin, J=5.7 Hz, 1H), 2.01-2.10 (m, 2H), 1.63-1.86 ppm (m, 6H)

Solution 1: {4-(4-pyridyloxy)cyclohexanol, 1 eq, 6.07 g} in {MeOH, 91.05 mL} and {THF, 30.35 mL}

The fixed bed (named FLR1, volume 20 mL) was completely packed with granular catalyst 10% Ru/SiO₂ (1.00 eq) 10.00 g. The H₂ back pressure regulator was adjusted to 2.5 MPa, and the flow rate of H₂ was 60 mL/min. Then the solution S1 was pumped by Pump 1 {S1, P1,1.212 mL/min} to fixed bed {FLR1, SS, Fixed bed, 9.525 (1/2″) mm, 20 mL, 95° C.}. Then the reaction mixture was collected from the reactor output. The fixed bed was washed by extra {MeOH, 91.05 mL}. Compound 4-(4-piperidyloxy)cyclohexanol (6.28 g, 31.51 mmol, 100% yield) was obtained as a colourless oil.

To a solution of 4-(4-piperidyloxy)cyclohexanol (6.28 g, 31.51 mmol, 1.0 eq) in THF (60 mL) and water (30 mL) was added NaHCO₃ (13.24 g, 157.56 mmol, 6.13 mL, 5.0 eq) and Boc₂O (10.32 g, 47.27 mmol, 10.86 mL, 1.5 eq). The mixture was stirred at 25° C. for 12 h. The mixture was poured into water (70 mL). The aqueous phase was extracted with ethyl acetate (100 mL×2). The combined organic phase was washed with brine (200 mL×3), dried with anhydrous sodium sulfate, filtered and concentrated in vacuum. The residue was purified by silica gel chromatography (0%-40% ethyl acetate in petroleum ether). Compound tert-butyl 4-(4-hydroxycyclohexoxy)piperidine-1-carboxylate (7.94 g, 26.52 mmol, 84% yield) was obtained as a colourless oil.

¹H NMR (400 MHz, CDCl₃) δ=3.74 (td, J=7.3, 3.6 Hz, 3H), 3.58-3.43 (m, 2H), 3.10 (ddd, J=13.2, 9.2, 3.5 Hz, 2H), 1.81-1.73 (m, 4H), 1.71 (s, 4H), 1.56-1.48 (m, 4H), 1.45 (s, 9H)

LC-MS: MS (ESI⁺) t_{R}=0.492 min, m/z=244.1 [M−55]

To a solution of tert-butyl 4-(4-hydroxycyclohexoxy)piperidine-1-carboxylate (7.94 g, 26.52 mmol, 1.0 eq) in dichloromethane (80 mL) was added Et₃N (21.47 g, 212.15 mmol, 29.53 mL, 8.0 eq) and MsCl (4.00 g, 34.92 mmol, 2.70 mL, 1.3 eq) at 0° C. The mixture was stirred at 25° C. for 3 h. The mixture was poured into iced water (200 mL). The aqueous phase was extracted with ethyl acetate (100 mL×2). The combined organic phase was washed with brine (200 mL×3), dried with anhydrous sodium sulfate, filtered and concentrated in vacuum The reaction mixture was concentrated under reduced pressure to give the product. The residue was purified by silica gel chromatography (0%-50% ethyl acetate in petroleum ether). Compound tert-butyl 4-(4-methylsulfonyloxycyclohexoxy)piperidine-1-carboxylate (9.24 g, 24.48 mmol, 92% yield) was obtained as a colourless oil.

¹H NMR (400 MHz, CDCl₃) δ=4.80-4.70 (m, 1H), 3.82-3.64 (m, 3H), 3.58-3.43 (m, 3H), 3.14-3.06 (m, 3H), 2.03-1.96 (m, 2H), 1.79-1.73 (m, 6H), 1.47-1.41 (m, 13H)

To a solution of 1-(3-iodo-1,4,6,7-tetrahydropyrazolo[4,3-c]pyridin-5-yl)ethanone (3.00 g, 10.31 mmol, 1.0 eq) and tert-butyl 4-(4-methylsulfonyloxycyclohexoxy)piperidine-1-carboxylate (4.67 g, 12.37 mmol, 1.2 eq) in DMF (35 mL) was added Cs₂CO₃ (10.07 g, 30.92 mmol, 3.0 eq). The reaction mixture was stirred at 110° C. for 12 h. The mixture was poured into water (150 mL). The aqueous phase was extracted with ethyl acetate (100 mL×2). The combined organic phase was washed with brine (200 mL×3), dried with anhydrous sodium sulfate, filtered and concentrated in vacuum. The residue was purified by prep-HPLC (column: Welch Ultimate XB—CN 250*70*10 um; mobile phase: [Hexane-EtOH]; gradient: 10%-50% B over 15 min) to give desired compound. Compound tert-butyl 4-[4-(5-acetyl-3-iodo-6,7-dihydro-4H-pyrazolo[4,3-c]pyridin-1-yl)cyclohexoxy]piperidine-1-carboxylate (660 mg, 1.15 mmol, 11% yield) was obtained as a white solid.

¹H NMR (400 MHz, CDCl₃) δ=4.41-4.24 (m, 2H), 3.91-3.85 (m, 2H), 3.79 (d, J=10.9 Hz, 2H), 3.71 (t, J=5.7 Hz, 1H), 3.60-3.53 (m, 1H), 3.50-3.41 (m, 1H), 3.06 (ddd, J=13.2, 9.6, 3.2 Hz, 2H), 2.76-2.65 (m, 2H), 2.19-2.17 (m, 3H), 2.14-2.08 (m, 2H), 2.03-1.98 (m, 1H), 1.96-1.89 (m, 2H), 1.79 (d, J=9.8 Hz, 2H), 1.66 (s, 1H), 1.53-1.40 (m, 13H)

LC-MS: MS (ESI⁺): t_{R}=0.558 min, m/z=573.1 [M+H⁺]

To a solution of tert-butyl 4-[4-(5-acetyl-3-iodo-6,7-dihydro-4H-pyrazolo[4,3-c]pyridin-1-yl)cyclohexoxy]piperidine-1-carboxylate (300 mg, 524.04 μmol, 1.0 eq), 7-(difluoromethyl)-6-(1-methylpyrazol-4-yl)-1,2,3,4-tetrahydroquinoline (206 mg, 786.06 μmol, 1.5 eq) in tert-amyl alcohol (5 mL) was added Cs₂CO₃ (512 mg, 1.57 mmol, 3.0 eq) and Cphos Pd G₃ (42 mg, 52.40 μmol, 0.1 eq). The mixture was stirred at 90° C. for 12 hours under N₂. The mixture was poured into water (70 mL). The aqueous phase was extracted with ethyl acetate (100 mL×2). The combined organic phase was washed with brine (200 mL×3), dried with anhydrous sodium sulfate, filtered and concentrated in vacuum. The residue was purified by prep-TLC (SiO₂, Dichloromethane:Methanol=15:1). Compound tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexoxy]piperidine-1-carboxylate (230 mg, 324.93 μmol, 62% yield) was obtained as a yellow solid.

LC-MS: MS (ESI⁺): t_{R}=0.574 min, m/z=708.4 [M+H⁺]

To a solution of tert-butyl 4-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexoxy]piperidine-1-carboxylate (115 mg, 162.46 μmol, 1.0 eq) in dichloromethane (3 mL) was added trifluoroacetic acid (4.61 g, 40.39 mmol, 3 mL, 248.6 eq). The mixture was stirred at 25° C. for 0.5 h. The reaction mixture was concentrated under reduced pressure to give the product. Compound 1-[3-[7-(difluoromethyl)-6-(1-methylpyrazol-4-yl)-3,4-dihydro-2H-quinolin-1-yl]-1-[4-(4-piperidyloxy)cyclohexyl]-6,7-dihydro-4H-pyrazolo[4,3-c]pyridin-5-yl]ethanone (98 mg) was obtained as a yellow oil and directly used in the next step without further purification.

LC-MS: MS (ESI⁺): t_{R}=0.442 min, m/z=608.3 [M+H⁺]

To a solution of 1-[3-[7-(difluoromethyl)-6-(1-methylpyrazol-4-yl)-3,4-dihydro-2H-quinolin-1-yl]-1-[4-(4-piperidyloxy)cyclohexyl]-6,7-dihydro-4H-pyrazolo[4,3-c]pyridin-5-yl]ethanone (98 mg) in NMP (3 mL) was added DIPEA (416 mg, 3.23 mmol, 561.74 μL, 20.0 eq) and 2-chloro-N-[3-(3-chloro-4-cyano-phenoxy)-2,2,4,4-tetramethyl-cyclobutyl]pyrimidine-5-carboxamide (67 mg, 161.25 μmol, 1.0 eq). The mixture was stirred at 50° C. for 12 h. The mixture was poured into water (50 mL). The aqueous phase was extracted with dichloromethane (50 mL×2). The combined organic phase was washed with brine (100 mL×3), dried with anhydrous sodium sulfate, filtered and concentrated in vacuum. The residue was purified by prep-TLC (SiO₂, Dichloromethane:Methanol=40:1) for twice. The residue was purified by prep-HPLC (column: Unisil 3-100 C18 Ultra 150*5 mm*3 um; mobile phase: [water (FA)-ACN]; gradient: 60%-90% B over 10 mi) to give desired compound. Then it was lyophilized. Compound 2-[4-[4-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]cyclohexoxy]-1-piperidyl]-N-[3-(3-chloro-4-cyano-phenoxy)-2, 2,4,4-tetramethyl-cyclobutyl]pyrimidine-5-carboxamide (20.19 mg, 19.95 μmol, 12.37% yield) was obtained as a white solid.

¹H NMR (400 MHz, CDCl₃) δ=8.70 (s, 2H), 7.67-7.49 (s, 2H), 7.41 (d, J=6.4 Hz, 1H), 7.08-6.95 (m, 2H), 6.88 (d, J=5.9 Hz, 1H), 6.81 (dd, J=8.8, 2.4 Hz, 1H), 6.70-6.35 (m, 1H), 5.93 (d, J=8.1 Hz, 1H), 4.33 (dt, J=13.1, 5.0 Hz, 2H), 4.26 (s, 1H), 4.15-4.08 (m, 2H), 4.05 (s, 1H), 3.96 (d, J=2.1 Hz, 3H), 3.94-3.85 (m, 2H), 3.82-3.63 (m, 4H), 3.62-3.46 (m, 3H), 2.99-2.84 (m, 2H), 2.80 (t, J=5.5 Hz, 1H), 2.74 (t, J=5.5 Hz, 1H), 2.19-2.13 (m, 3H), 2.13-2.07 (m, 2H), 2.07-1.95 (m, 6H), 1.91 (td, J 6.4, 3.1 Hz, 2H), 1.63-1.60 (d, 2H), 1.51-1.42 (m, 2H), 1.24 (d, J=13.1 Hz, 12H)

LC-MS: MS (ESI⁺): t_{R}=3.280 min, m/z=990.7 [M+H⁺]

The compounds below were prepared in a similar manner as described in Example 54.

| | | | LC-MS |
|---|---|---|---|
| Compound | Name | ¹H NMR (CDCl₃) | [M + H⁺] |
| 338 | 2-[4-[4-[5-acetyl-3-[7- | δ = 8.70 (s, 2H), 7.61-7.48 (m, 2H), | 990.6 |
| | (difluoromethyl)-6-(1- | 7.45-7.34 (m, 1H), 7.08-6.94 (m, 2H), | |
| | methylpyrazol-4-yl)-3,4- | 6.92-6.85 (m, 1H), 6.81 (dd, J = 2.4, 8.8 | |
| | dihydro-2H-quinolin-1-yl]-6,7- | Hz, 1H), 6.71-6.31 (m, 1H), 5.96 (d, J = | |
| | dihydro-4H-pyrazolo[4,3- | 9.2 Hz, 1H), 4.34-4.18 (m, 3H), 4.15- | |
| | c]pyridin-1-yl]cyclohexoxy]-1- | 4.11 (m, 2H), 4.04 (s, 1H), 3.95 (d, J = | |
| | piperidyl]-N-[3-(3-chloro-4- | 2.4 Hz, 4H), 3.90 (t, J = 5.6 Hz, 1H), | |
| | cyano-phenoxy)-2,2,4,4- | 3.78-3.64 (m, 7H), 2.93-2.71 (m, 4H), | |
| | tetramethyl- | 2.44-2.26 (m, 2H), 2.16 (s, 1H), 2.10- | |
| | cyclobutyl]pyrimidine-5- | 2.01 (m, 6H), 1.93-1.85 (m, 2H), 1.76 ( | |
| | carboxamide | d, J = 12.8 Hz, 3H), 1.69-1.61 (m, 3H), | |
| | | 1.61-1.52 (m, 2H), 1.23 (d, J = 12.0 Hz, | |
| | | 10H) | |
| 340 | 2-[3-[4-[5-acetyl-3-[7- | δ = 8.71 (s, 2H), 7.62-7.50 (m, 2H), | 962.7 |
| | (difluoromethyl)-6-(1- | 7.41 (d, J = 6.4 Hz, 1H), 7.03 (br d, J = | |
| | methylpyrazol-4-yl)-3,4- | 21.6 Hz, 1H), 6.97 (d, J = 2.4 Hz, 1H), | |
| | dihydro-2H-quinolin-1-yl]-6,7- | 6.87 (d, J = 6.0 Hz, 1H), 6.81 (dd, J = | |
| | dihydro-4H-pyrazolo[4,3- | 2.4, 8.8 Hz, 1H), 6.53 (dt, J = 10.4, 55.6 | |
| | c]pyridin-1- | Hz, 1H), 5.97-5.88 (m, 1H), 4.62-4.51 | |
| | yl]cyclohexoxy]azetidin-1-yl]- | (m, 1H), 4.50-4.38 (m, 2H), 4.26 (s, | |
| | N-[3-(3-chloro-4-cyano- | 1H), 4.14-4.09 (m, 4H), 4.05 (s, 1H), | |
| | phenoxy)-2,2,4,4-tetramethyl- | 3.96 (d, J = 2.0 Hz, 3H), 3.94-3.88 (m, | |
| | cyclobutyl]pyrimidine-5- | 2H), 3.80-3.65 (m, 3H), 3.52-3.39 (m, | |
| | carboxamide | 1H), 2.87 (td, J = 6.0, 11.9 Hz, 2H), 2.83- | |
| | | 2.77 (m, 1H), 2.74 (br t, J = 5.6 Hz, | |
| | | 1H), 2.19-2.13 (m, 3H), 2.12-1.98 (m, | |
| | | 8H), 1.56-1.42 (m, 3H), 1.24 (d, J = | |
| | | 13.2 Hz, 12H) | |
| 308 | 4-[4-[4-[5-acetyl-3-[7- | δ = 8.04-7.99 (m, 1H), 7.72-7.64 (m, | 984.7 |
| | (difluoromethyl)-6-(1- | 2H), 7.57-7.51 (m, 1H), 7.49-7.37 (m, | |
| | methylpyrazol-4-yl)-3,4- | 2H), 7.07-6.97 (m, 1H), 6.96-6.84 (m, | |
| | dihydro-2H-quinolin-1-yl]-6,7- | 3H), 6.75-6.34 (m, 3H), 6.13 (d, J = 8.0 | |
| | dihydro-4H-pyrazolo[4,3- | Hz, 1H), 4.25 (s, 1H), 4.17-4.12 (m, | |
| | c]pyridin-1-yl]cyclohexoxy]-1- | 2H), 4.05 (s, 1H), 3.98-3.86 (m, 8H), | |
| | piperidyl]-N-[3-(4-cyano-3- | 3.83-3.68 (m, 4H), 3.62 (d, J = 3.6 Hz, | |
| | methoxy-phenoxy)-2,2,4,4- | 3H), 3.14 (t, J = 9.2 Hz, 2H), 2.96 (s, | |
| | tetramethyl- | 1H), 2.91-2.79 (m, 4H), 2.78-2.74 (m, | |
| | cyclobutyl]benzamide | 1H), 2.39-2.26 (m, 2H), 2.16 (s, 1H), | |
| | | 2.12-1.99 (m, 6H), 1.98-1.91 (m, 2H), | |
| | | 1.83 (s, 2H), 1.61-1.52 (m, 2H), 1.25 (d, | |
| | | J = 10.0 Hz, 12H) | |
| 309 | 4-[4-[4-[5-acetyl-3-[7- | δ = 7.74-7.63 (m, 2H), 7.58-7.51 (m, | 984.7 |
| | (difluoromethyl)-6-(1- | 1H), 7.49-7.43 (m, 1H), 7.43-7.38 (m, | |
| | methylpyrazol-4-yl)-3,4- | 1H), 7.08-6.98 (m, 1H), 6.93 (d, J = 8.8 | |
| | dihydro-2H-quinolin-1-yl]-6,7- | Hz, 2H), 6.87 (d, J = 6.0 Hz, 1H), 6.73- | |
| | dihydro-4H-pyrazolo[4,3- | 6.42 (m, 2H), 6.41-6.33 (m, 1H), 6.18- | |
| | c]pyridin-1-yl]cyclohexoxy]-1- | 6.04 (m, 1H), 4.26 (s, 1H), 4.18-4.10 | |
| | piperidyl]-N-[3-(4-cyano-3- | (m, 2H), 4.05 (s, 1H), 3.96 (d, J = 2.0 | |
| | methoxy-phenoxy)-2,2,4,4- | Hz, 3H), 3.93-3.90 (m, 4H), 3.78-3.60 | |
| | tetramethyl- | (m, 6H), 3.56-3.45 (m, 1H), 3.09 (t, J = | |
| | cyclobutyl]benzamide | 10.0 Hz, 2H), 2.91-2.72 (m, 4H), 2.17 | |
| | | (s, 3H), 2.09-1.93 (m, 9H), 1.75-1.66 | |
| | | (m, 3H), 1.54-1.39 (m, 3H), 1.25 (d, J = | |
| | | 10.0 Hz, 12H) | |
| 341 | -[3-[4-[5-acetyl-3-[7- | δ = 8.72 (s, 2H), 7.59-7.52 (m, 2H), | 962.6 |
| | (difluoromethyl)-6-(1- | 7.43-7.38 (m, 1H), 7.05-6.98 (m, 1H), | |
| | methylpyrazol-4-yl)-3,4- | 6.97 (d, J = 2.4 Hz, 1H), 6.91-6.86 (m, | |
| | dihydro-2H-quinolin-1-yl]-6,7- | 1H), 6.81 (dd, J = 2.4, 8.7 Hz, 1H), 6.69- | |
| | dihydro-4H-pyrazolo[4,3- | 6.35 (m, 1H), 6.06-5.93 (m, 1H), 4.58- | |
| | c]pyridin-1- | 4.49 (m, 1H), 4.43 (dd, J = 6.4, 9.9 Hz, | |
| | yl]cyclohexoxyJazetidin-1-yl]- | 2H), 4.26 (s, 1H), 4.17 (dd, J = 4.4, 10.7 | |
| | N-[3-(3-chloro-4-cyano- | Hz, 2H), 4.13 (t, J = 3.9 Hz, 2H), 4.05 (s, | |
| | phenoxy)-2,2,4,4-tetramethyl- | 1H), 3.99-3.92 (m, 4H), 3.90 (t, J = 5.8 | |
| | cyclobutyl]pyrimidine-5- | Hz, 1H), 3.79-3.65 (m, 4H), 2.93-2.78 | |
| | carboxamide | (m, 3H), 2.75 (t, J = 5.7 Hz, 1H), 2.39- | |
| | | 2.29 (m, 2H), 2.17 (s, 1H), 2.11-2.02 | |
| | | (m, 6H), 1.81-1.77 (m, 2H), 1.62 (d, J = | |
| | | 14.0 Hz, 2H), 1.24 (d, J = 14.4 Hz, | |
| | | 12H) | |
| 342 | 4-[3-[4-[5-acetyl-3-[7- | δ 7.67 (d, J = 8.50 Hz, 2 H), 7.67 (d, | 956.6 |
| | (difluoromethyl)-6-(1- | J = 8.50 Hz, 2 H), 7.54 (d, J = 5.00 Hz, 1 | |
| | methylpyrazol-4-yl)-3,4- | H), 7.45 (d, J = 8.50 Hz, 1 H), 7.41 (d, | |
| | dihydro-2H-quinolin-1-yl]-6,7- | J = 6.13 Hz, 1 H), 7.03 (d, J = 21.51 Hz, 1 | |
| | dihydro-4H-pyrazolo[4,3- | H), 6.87 (d, J = 6.25 Hz, 1 H), 6.68-6.38 | |
| | c]pyridin-1- | (m, 5 H), 6.10 (d, J = 8.00 Hz, 1 H), 4.58 | |
| | yl]cyclohexoxy]azetidin-1-yl]- | (q, J = 5.50 Hz, 1 H), 4.31-4.17 (m, 3 H), | |
| | N-[3-(4-cyano-3-methoxy- | 4.16-4.12 (m, 2 H), 4.05 (s, 1 H), 3.96 (d, | |
| | phenoxy)-2,2,4,4-tetramethyl- | J = 1.75 Hz, 3 H), 3.94-3.88 (m, 5 H), | |
| | cyclobutyl]benzamide | 3.79 (dd, J = 7.94, 4.69 Hz, 2 H), 3.77- | |
| | | 3.64 (m, 3 H), 3.51-3.37 (m, 1 H), 2.88 | |
| | | (dt, J = 11.82, 6.10 Hz, 2 H), 2.83-2.71 | |
| | | (m, 2 H), 2.18-2.13 (m, 3 H), 2.12-1.96 | |
| | | (m, 8 H), 1.51-1.41 (m, 2 H) | |
| | | 1.25 (d, J = 9.88 Hz, 12 H) | |
| 343 | 4-[3-[4-[5-acetyl-3-[7- | δ = 7.66 (d, J = 8.5 Hz, 2H), 7.56-7.51 | 956.7 |
| | (difluoromethyl)-6-(1- | (m, 1H), 7.45 (d, J = 8.5 Hz, 1H), 7.43- | |
| | methylpyrazol-4-yl)-3,4- | 7.38 (m, 1H), 7.04 (s, 1H), 6.90-6.84 | |
| | dihydro-2H-quinolin-1-yl]-6,7- | (m, 1H), 6.70-6.49 (m, 1H), 6.48-6.45 | |
| | dihydro-4H-pyrazolo[4,3- | (m, 2H), 6.44 (s, 1H), 6.42-6.38 (m, | |
| | c]pyridin-1- | 1H), 6.10 (d, J = 7.9 Hz, 1H), 4.59-4.49 | |
| | yl]cyclohexoxy]azetidin-1-yl]- | (m, 1H), 4.25 (s, 1H), 4.20 (t, J = 7.2 Hz, | |
| | N-[3-(4-cyano-3-methoxy- | 2H), 4.15 (d, J = 8.3 Hz, 1H), 4.12 (s, | |
| | phenoxy)-2,2,4,4-tetramethyl- | 1H), 4.05 (s, 1H), 3.96 (d, J = 1.6 Hz, | |
| | cyclobutyl]benzamide | 4H), 3.92 (s, 3H), 3.91-3.88 (m, 1H), | |
| | | 3.85 (dd, J = 4.9, 7.6 Hz, 2H), 3.78-3.64 | |
| | | (m, 4H), 2.94-2.84 (m, 2H), 2.84-2.78 | |
| | | (m, 1H), 2.75 (br t, J = 5.5 Hz, 1H), 2.38- | |
| | | 2.25 (m, 2H), 2.17 (s, 1H), 2.08 (br d, | |
| | | J = 5.5 Hz, 2H), 2.05 (s, 4H), 1.78 (d, J = | |
| | | 10.8 Hz, 2H), 1.56 (s, 2H), 1.25 (d, J = | |
| | | 9.8 Hz, 12H) | |

### Example 55: Synthesis of 2-[6-[4-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]-1-piperidyl]-2-azaspiro[3.4]octan-2-yl]-N-[3-(3-chloro-4-cyano-phenoxy)-2,2,4,4-tetramethyl-cyclobutyl]pyrimidine-5-carboxamide (Compound 344)

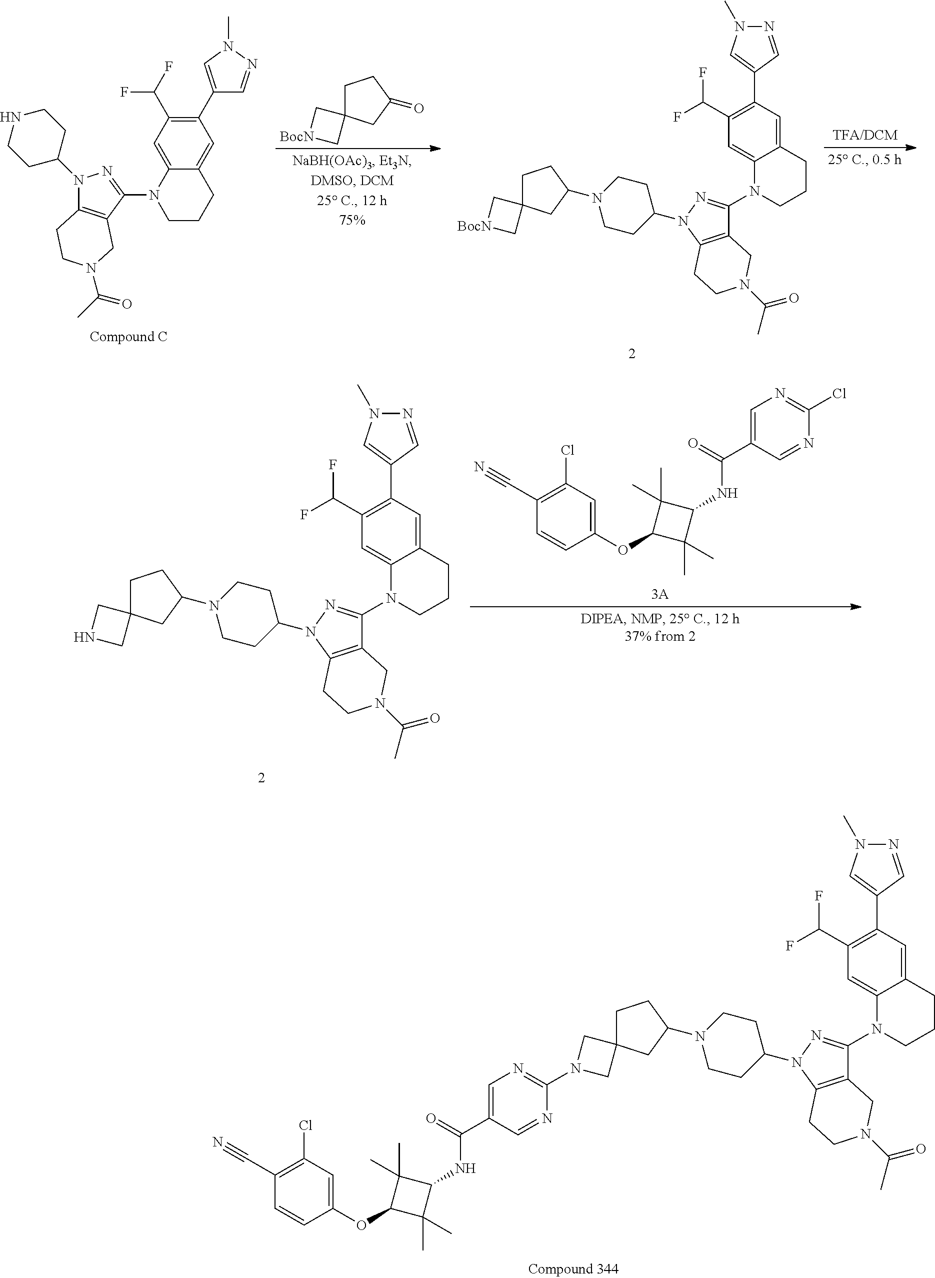

To a solution of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (300 mg, 549 μmol, 1.0 eq, HCl salt), tert-butyl 6-oxo-2-azaspiro[3.4]octane-2-carboxylate (247 mg, 1.10 mmol, 2.0 eq) in a mixture of DCM (4 mL) and DMSO (2 mL) was added Et₃N (166 mg, 1.65 mmol, 229 μL, 3.0 eq). The reaction mixture was stirred at 25° C. for 0.5 h, and then NaBH(OAc)₃ (582 mg, 2.75 mmol, 5.0 eq) was added. The reaction was stirred at 25° C. for 11.5 h. To the reaction mixture was added water (30 mL) and the mixture was extracted with DCM (30 mL×3). The combined organic phase was washed with brine (20 mL), dried over anhydrous Na₂SO₄, filtered, and concentrated. The residue was purified by column chromatography (SiO₂, DCM/MeOH=30/1 to 10/1). Compound tert-butyl 6-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.4]octane-2-carboxylate (300 mg, 417 mol, 75% yield) was obtained as a yellow solid.

LC-MS: MS (ESI⁺): t_{R}=0.534 min, m/z=719.4 [M+H⁺]

To a solution of tert-butyl 6-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.4]octane-2-carboxylate (300 mg, 417 mol, 1.0 eq) in DCM (6 mL) was added TFA (3.07 g, 26.9 mmol, 2 mL, 64.5 eq). The mixture was stirred at 25° C. for 0.5 h. The reaction mixture was adjusted to pH=8 with saturated NaHCO₃ and the mixture was extracted with DCM (30 mL×3). The combined organic phase was washed with brine (20 mL), dried over anhydrous Na₂SO₄, filtered, and concentrated under reduce pressure. Compound 1-[1-[1-(2-azaspiro[3.4]octan-6-yl)-4-piperidyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (260 mg) was obtained as a brown solid and directly used in the next step without further purification.

LC-MS: MS (ESI⁺): t_{R}=0.469 min, m/z=619.3 [M+H⁺]

To a solution of 1-[1-[1-(2-azaspiro[3.4]octan-6-yl)-4-piperidyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (80 mg), 2-chloro-N-[3-(3-chloro-4-cyano-phenoxy)-2,2,4,4-tetramethyl-cyclobutyl]pyrimidine-5-carboxamide (54 mg, 129 μmol, 1.0 eq) in NMP (1 mL) was added DIPEA (84 mg, 646 μmol, 112 μL, 5.0 eq). The mixture was stirred at 25° C. for 12 h. The reaction mixture was purified by preparative HPLC (column: Phenomenex Luna C18 150*25 mm*10 um; mobile phase: [water(FA)-ACN]; gradient: 33%-63% B over 10 min). Compound 2-[6-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-2-azaspiro[3.4]octan-2-yl]-N-[3-(3-chloro-4-cyano-phenoxy)-2,2,4,4-tetramethyl-cyclobutyl]pyrimidine-5-carboxamide (48.23 mg, 48 μmol, 37% yield over two steps) was obtained as a yellow solid.

¹H NMR (400 MHz, CDCl₃): δ 8.70 (s, 2H), 8.42 (s, 1H), 7.57 (d, J=8.8 Hz, 1H), 7.54 (d, J=4.8 Hz, 1H), 7.41 (d, J=5.2 Hz, 1H), 7.04 (d, J=18.0 Hz, 1H), 6.99-6.88 (m, 2H), 6.84-6.77 (m, 1H), 6.73-6.36 (m, 1H), 6.06-5.93 (m, 1H), 4.32 (s, 1H), 4.21-4.07 (m, 5H), 4.05 (s, 1H), 4.01 (s, 2H), 3.96 (s, 3H), 3.91 (t, J=5.6 Hz, 1H), 3.76 (t, J=5.6 Hz, 1H), 3.72-3.63 (m, 2H), 3.46-3.28 (m, 2H), 3.15-2.95 (m, 2H), 2.92-2.69 (m, 7H), 2.25 (d, J=7.6 Hz, 4H), 2.18 (s, 2H), 2.12-2.04 (m, 5H), 2.00-1.88 (m, 2H), 1.26 (s, 6H), 1.22 (s, 6H).

LC-MS: MS (ESI⁺): t_{R}=1.96 min, m/z=1001.6 [M+H⁺]

The compounds below were prepared in a similar manner as described in Example 55.

| | | | LC-MS |
|---|---|---|---|
| Compound | Name | ¹H NMR (DMSO-d₆) | [M + H⁺] |
| 345 | 4-[6-[4-[5-acetyl-3-[7- | (CDCl₃) δ 7.66 (d, J = 8.8 Hz, 2H), 7.54 | 995.2 |
| | (difluoromethyl)-6-(1- | (d, J = 4.8 Hz, 1H), 7.45 (d, J = 8.8 Hz, | |
| | methylpyrazol-4-yl)-3,4- | 1H), 7.41 (d, J = 6.0 Hz, 1H), 7.04 (d, | |
| | dihydro-2H-quinolin-1-yl]-6,7- | J = 18.0 Hz, 1H), 6.97-6.84 (m, 1H), 6.72- | |
| | dihydro-4H-pyrazolo[4,3- | 6.51 (m, 1H), 6.47 (d, J = 2.0 Hz, 1H), | |
| | c]pyridin-1-yl]-1-piperidyl]-2- | 6.45-6.36 (m, 3H), 6.09 (d, J = 8.0 Hz, | |
| | azaspiro[3.4]octan-2-yl]-N-[3- | 1H), 4.30 (s, 1H), 4.16-4.12 (m, 2H), | |
| | (4-cyano-3-methoxy-phenoxy)- | 4.05 (s, 1H), 3.96 (d, J = 1.2 Hz, 3H), | |
| | 2,2,4,4-tetramethyl- | 3.94-3.89 (m, 5H), 3.87-3.83 (m, 1H), | |
| | cyclobutyl]benzamide | 3.77 (s, 2H), 3.73-3.66 (m, 2H), 3.40- | |
| | | 3.21 (m, 2H), 2.91-2.85 (m, 2H), 2.82 | |
| | | (d, J = 5.6 Hz, 1H), 2.74 (t, J = 5.6 Hz, | |
| | | 1H), 2.49-2.38 (m, 1H), 2.31-2.21 (m, | |
| | | 4H), 2.19-1.97 (m, 12H), 1.95-1.87 | |
| | | (m, 2H), 1.85-1.74 (m, 1H), 1.25 (d, J = | |
| | | 10.0 Hz, 12H) | |
| 346 | 2-[2-[4-[5-acetyl-3-[7- | δ = 8.75 (s, 2H), 8.15-8.12 (m, 1H), | 1001.4 |
| | (difluoromethyl)-6-(1- | 7.93-7.86 (m, 1H), 7.75 (s, 1H), 7.73- | |
| | methylpyrazol-4-yl)-3,4- | 7.64 (m, 1H), 7.50 (s, 1H), 7.25-7.18 | |
| | dihydro-2H-quinolin-1-yl]-6,7- | (m, 1H), 7.11 (s, 1H), 7.05-6.97 (m, | |
| | dihydro-4H-pyrazolo[4,3-c] | 1H), 6.95-6.64 (m, 2H), 6.33-6.31 (m, | |
| | pyridin-1-yl]-1-piperidyl]-6- | 1H), 4.36-4.25 (m, 1H), 4.19-4.00 (m, | |
| | azaspiro [3.4] octan-6-yl]-N-[3- | 4H), 3.86 (s, 3H), 3.76-3.66 (m, 2H), | |
| | (3-chloro-4-cyano-phenoxy)- | 3.62-3.55 (m, 4H), 3.55-3.44 (m, 3H), | |
| | 2,2,4,4-tetramethyl-cyclobutyl] | 3.02-2.91 (m, 2H), 2.91-2.71 (m, 5H), | |
| | pyrimidine-5-carboxamide | 2.13 (s, 2H), 2.09-1.87 (m, 14H), 1.24- | |
| | | 1.17 (m, 6H), 1.13-1.04 (m, 6H) | |
| 347 | 4-[2-[4-[5-acetyl-3-[7- | δ = 8.22 (s, 1H), 7.83-7.70 (m, 3H), | 995.4 |
| | (difluoromethyl)-6-(1- | 7.68-7.60 (m, 1H), 7.50 (s, 1H), 7.45- | |
| | methylpyrazol-4-yl)-3,4- | 7.31 (m, 1H), 7.15-7.07 (m, 1H), 6.97- | |
| | dihydro-2H-quinolin-1-yl]-6,7- | 6.62 (m, 3H), 6.60-6.47 (m, 3H), 4.32- | |
| | dihydro-4H-pyrazolo[4,3- | 4.24 (m, 1H), 4.19-4.10 (m, 2H), 4.09- | |
| | c]pyridin-1-yl]-1-piperidyl]-6- | 3.99 (m, 2H), 3.91 (s, 3H), 3.89-3.84 | |
| | azaspiro[3.4]octan-6-yl]-N-[3- | (m, 3H), 3.76-3.68 (m, 2H), 3.61-3.56 | |
| | (4-cyano-3-methoxy-phenoxy)- | (m, 2H), 3.36-3.32 (m, 2H), 3.28-3.22 | |
| | 2,2,4,4-tetramethyl- | (m, 2H), 2.93-2.70 (m, 7H), 2.15-2.05 | |
| | cyclobutyl]benzamide | (m, 4H), 2.05-1.93 (m, 7H), 1.92-1.80 | |
| | | (m, 6H), 1.28-1.19 (m, 6H), 1.18-1.10 | |
| | | (m, 6H) | |
| 348 | 2-[6-[4-[5-acetyl-3-[7- | δ = 8.73 (s, 2H), 7.90 (d, J = 8.4 Hz, 1H), | 987.4 |
| | (difluoromethyl)-6-(1- | 7.82-7.72 (m, 2H), 7.51 (s, 1H), 7.22 (d, | |
| | methylpyrazol-4-yl)-3,4- | J = 2.4 Hz, 1H), 7.11 (s, 1H), 7.05-6.99 | |
| | dihydro-2H-quinolin-1-yl]-6,7- | (m, 1H), 6.97-6.62 (m, 2H), 4.29 (s, | |
| | dihydro-4H-pyrazolo[4,3-c] | 1H), 4.22-4.09 (m, 4H), 4.08-3.99 (m, | |
| | pyridin-1-yl]-1-piperidyl]-2- | 4H), 3.87 (s, 3H), 3.78-3.67 (m, 2H), | |
| | azaspiro [3.3] heptan-2-yl]-N- | 3.63-3.56 (m, 2H), 2.94-2.82 (m, 5H), | |
| | [3-(3-chloro-4-cyano-phenoxy)- | 2.76-2.64 (m, 2H), 2.40-2.32 (m, 2H), | |
| | 2,2,4,4-tetramethyl-cyclobutyl] | 2.11-2.04 (m, 3H), 2.03-1.94 (m, 6H), | |
| | pyrimidine-5-carboxamide | 1.93-1.83 (m, 4H), 1.21 (s, 6H), 1.11 (s, | |
| | | 6H) | |
| 349 | 4-[6-[4-[5-acetyl-3-[7- | δ = 7.79-7.68 (m, 3H), 7.67-7.62 (m, | 981.7 |
| | (difluoromethyl)-6-(1- | 1H), 7.50 (s, 1H), 7.45 (d, J = 8.8 Hz, | |
| | methylpyrazol-4-yl)-3,4- | 1H), 7.10 (s, 1H), 6.94-6.62 (m, 3H), | |
| | dihydro-2H-quinolin-1-yl]-6,7- | 6.58-6.49 (m, 1H), 6.40 (d, J = 8.4 Hz, | |
| | dihydro-4H-pyrazolo[4,3- | 2H), 4.26 (s, 1H), 4.20-4.09 (m, 2H), | |
| | c]pyridin-1-yl]-1-piperidyl]-2- | 4.04 (d, J = 9.2 Hz, 2H), 3.90 (s, 5H), | |
| | azaspiro[3.3]heptan-2-yl]-N-[3- | 3.87 (s, 3H), 3.79 (s, 2H), 3.75-3.66 (m, | |
| | (4-cyano-3-methoxy-phenoxy)- | 2H), 3.62-3.56 (m, 2H), 2.91-2.81 (m, | |
| | 2,2,4,4-tetramethyl- | 5H), 2.76-2.61 (m, 2H), 2.33 (m, J = 8.4 | |
| | cyclobutyl]benzamide | Hz, 2H), 2.07 (s, 2H), 2.05-1.92 (m, | |
| | | 7H), 1.87 (m, J = 11.2 Hz, 4H), 1.21 (s, | |
| | | 6H), 1.17-1.05 (m, 6H). | |
| 426 | 4-[7-[4-[5-acetyl-3-[7- | δ = 7.81-7.70 (m, 2H), 7.71-7.55 (m, | 1009.8 |
| | (difluoromethyl)-6-(1- | 3H), 7.48-7.45 (m, 1H), 7.41-7.30 (m, | |
| | methylimidazol-4-yl)-3,4- | 2H), 6.86-6.75 (m, 1H), 6.69-6.62 (m, | |
| | dihydro-2H-quinolin-1-yl]-6,7- | 1H), 6.60-6.51 (m, 1H), 6.48-6.36 (m, | |
| | dihydro-4H-pyrazolo[4,3- | 2H), 4.27 (s, 1H), 4.21-4.09 (m, 2H), | |
| | c]pyridin-1-yl]-1-piperidyl]-2- | 4.09-4.04 (m, 1H), 4.05-3.85 (m, 3H), | |
| | azaspiro[3.5]nonan-2-yl]-N-[3- | 3.80-3.66 (m, 4H), 3.65-3.56 (m, 4H), | |
| | (4-cyano-3-methoxy-phenoxy)- | 3.56-3.52 (m, 3H), 3.02-2.91 (m, 2H), | |
| | 2,2,4,4-tetramethyl- | 2.85 (s, 3H), 2.78-2.62 (m, 1H), 2.41- | |
| | cyclobutyl]benzamide | 2.22 (m, 3H), 2.08 (s, 2H), 2.05-1.80 | |
| | | (m, 10H), 1.75-1.65 (m, 2H), 1.56- | |
| | | 1.46 (m, 2H), 1.41-1.28 (m, 2H), 1.22 | |
| | | (m, 6H), 1.15 (m, 6H) | |
| 428 | 4-[7-[4-[5-acetyl-3-[7- | δ = 9.19 (s, 1H), 7.85-7.71 (m, 3H), 7.68- | 1012.8 |
| | (difluoromethyl)-6-thiazol-5-yl- | 7.61 (m, 1H), 7.46-7.39 (m, 1H), 7.22- | |
| | 3,4-dihydro-2H-quinolin-1-yl]- | 7.15 (m, 1H), 7.01-6.78 (m, 2H), 6.72- | |
| | 6,7-dihydro-4H-pyrazolo[4,3- | 6.61 (m, 1H), 6.57-6.51 (m, 1H), 6.48- | |
| | c]pyridin-1-yl]-1-piperidyl]-2- | 6.36 (m, 2H), 4.27 (s, 1H), 4.25-4.14 | |
| | azaspiro[3.5]nonan-2-yl]-N-[3- | (m, 2H), 4.10-3.95 (m, 2H), 3.91 (s, | |
| | (4-cyano-3-methoxy-phenoxy)- | 3H), 3.81-3.66 (m, 2H), 3.66-3.56 (m, | |
| | 2,2,4,4-tetramethyl- | 4H), 3.55 (s, 3H), 3.02-2.81 (m, 5H), | |
| | cyclobutyl]benzamide | 2.81-2.62 (m, 1H), 2.45-2.26 (m, 3H), | |
| | | 2.08 (s, 2H), 2.05-1.85 (m, 8H), 1.78- | |
| | | 1.64 (m, 2H), 1.56-1.47 (m, 2H), 1.41- | |
| | | 1.30 (m, 2H), 1.22 (m, 6H), 1.15 (m, 6H) | |
| 431 | 4-[7-[4-[5-acetyl-3-[7- | (CDCl₃) δ = 8.47-8.39 (m, 1H), 8.26- | 996.7 |
| | (difluoromethyl)-6-oxazol-5-yl- | 8.16 (m, 1H), 7.78-7.70 (m, 2H), 7.68- | |
| | 3,4-dihydro-2H-quinolin-1-yl]- | 7.63 (m, 1H), 7.48-7.39 (m, 2H), 7.34- | |
| | 6,7-dihydro-4H-pyrazolo[4,3- | 7.27 (m, 1H), 7.24-6.91 (m, 1H), 6.91- | |
| | c] pyridin-1-yl]-1-piperidyl]-2- | 6.86 (m, 1H), 6.67-6.61 (m, 1H), 6.57- | |
| | azaspiro[3.5]nonan-2-yl]-N-[3- | 6.51 (m, 1H), 6.46-6.38 (m, 2H), 4.27 | |
| | (4-cyano-3-methoxy-phenoxy)- | (s, 1H), 4.22-4.14 (m, 2H), 4.09-3.96 | |
| | 2,2,4,4-tetramethyl- | (m, 2H), 3.91 (s, 3H), 3.79-3.58 (m, | |
| | cyclobutyl]benzamide | 7H), 3.57-3.50 (m, 3H), 2.94 (br d, J = | |
| | | 7.5 Hz, 2H), 2.88 (br d, J = 5.4 Hz, 3H), | |
| | | 2.38-2.32 (m, 2H), 2.13-2.06 (m, 2H), | |
| | | 2.04-1.91 (m, 7H), 1.90-1.83 (m, 2H), | |
| | | 1.71 (br d, J = 9.9 Hz, 2H), 1.57-1.46 | |
| | | (m, 2H), 1.41-1.29 (m, 2H), 1.22 (s, | |
| | | 6H), 1.19-1.12 (m, 6H) | |
| 432 | 4-[7-[4-[5-acetyl-3-[7- | (CDCl₃) δ = 7.79-7.75 (m, 3H), 7.67- | 1009.8 |
| | (difluoromethyl)-6-(1- | 7.61 (m, 1H), 7.51-7.23 (m, 3H), 6.83 | |
| | methylpyrazol-3-yl)-3,4- | (s, 1H), 6.66-6.61 (m, 1H), 6.57-6.51 | |
| | dihydro-2H-quinolin-1-yl]-6,7- | (m, 1H), 6.49-6.45 (m, 1H), 6.44-6.35 | |
| | dihydro-4H-pyrazolo[4,3- | (m, 2H), 4.26 (s, 1H), 4.21-4.10 (m, | |
| | c]pyridin-1-yl]-1-piperidyl]-2- | 2H), 4.09-3.92 (m, 2H), 3.86 (s, 3H), | |
| | azaspiro[3.5]nonan-2-yl]-N-[3- | 3.68 (s, 3H), 3.80-3.65 (m, 2H), 3.65- | |
| | (4-cyano-3-methoxy-phenoxy)- | 3.56 (m, 4H), 3.44 (s, 2H), 3.02-2.91 | |
| | 2,2,4,4-tetramethyl- | (m, 2H), 2.86 (s, 3H), 2.76-2.62 (m, | |
| | cyclobutyl]benzamide | 1H), 2.36-2.23 (m, 3H), 2.07 (s, 2H), | |
| | | 2.04-1.90 (m, 7H), 1.90-1.80 (m, | |
| | | 2H), 1.75-1.62 (m, 2H), 1.58-1.45 (m, | |
| | | 2H), 1.38-1.22 (m, 3H), 1.21 (s, 6H), | |
| | | 1.14 (s, 6H) | |
| 448 | 4-[7-[4-[5-acetyl-3-[3-(1- | δ = 9.86 (s, 1H), 8.36 (s, 1H), 8.17-8.16 | 955.8 |
| | methylpyrazol-4-yl)-8- | (m, 1H), 8.09 (d, J = 2.8 Hz, 2H), 7.89- | |
| | isoquinolyl]-6,7-dihydro-4H- | 7.85 (m, 1H), 7.79 (brs, 1H), 7.73 (d, J = | |
| | pyrazolo[4,3-c] pyridin-1-yl]-1- | 8.4 Hz, 2H), 7.64 (d, J = 8.6 Hz, 1H), | |
| | piperidyl]-2-azaspiro [3.5] | 7.58-7.40 (m, 2H), 6.63 (d, J = 2.0 Hz, | |
| | nonan-2-yl]-N-[3-(4-cyano-3- | 1H), 6.53 (dd, J = 2.0, 8.7 Hz, 1H), 6.42 | |
| | methoxy-phenoxy)-2,2,4,4- | (d, J = 8.6 Hz, 2H), 4.57 (br s, 2H), 4.27 | |
| | tetramethyl-cyclobutyl] | (s, 1H), 4.22-4.12 (m, 1H), 4.05 (d, J = | |
| | benzamide | 9.2 Hz, 1H), 3.91 (s, 6H), 3.81 (br d, J = | |
| | | 5.4 Hz, 2H), 3.63 (s, 2H), 3.55 (s, 2H), | |
| | | 3.02 (br d, J = 6.2 Hz, 2H), 2.94 (br s, | |
| | | 1H), 2.88-2.74 (m, 1H), 2.44-2.36 (m, | |
| | | 3H), 2.15-2.07 (m, 4H), 2.03-1.93 (m, | |
| | | 5H), 1.79-1.70 (m, 2H), 1.53 (brs, 2H), | |
| | | 1.37 (br d, J = 11.6 Hz, 2H), 1.22 (s, 6H), | |
| | | 1.14 (s, 6H) | |
| 449 | 4-[7-[4-[5-acetyl-3-[7-methyl- | δ = 7.81-7.69 (m, 3H), 7.65 (d, J = 8.4 | 973.7 |
| | 6-(1-methylpyrazol-4-yl)-3,4- | Hz, 1H), 7.52 (s, 1H), 7.43 (d, J = 9.6 | |
| | dihydro-2H-quinolin-1-yl]-6,7- | Hz, 1H), 7.01-6.95 (m, 1H), 6.63 (d, J = | |
| | dihydro-4H-pyrazolo[4,3- | 2.4 Hz, 1H), 6.57-6.50 (m, 1H), 6.41 (d, | |
| | c]pyridin-1-yl]-1-piperidyl]-2- | J = 8.8 Hz, 2H), 6.35-6.28 (m, 1H), | |
| | azaspiro[3.5]nonan-2-yl]-N-[3- | 4.26 (s, 1H), 4.15-4.07 (m, 2H), 4.05 (d, | |
| | (4-cyano-3-methoxy-phenoxy)- | J = 9.2 Hz, 1H), 4.01-3.93 (m, 1H), | |
| | 2,2,4,4-tetramethyl- | 3.90 (s, 3H), 3.84 (s, 3H), 3.77-3.68 (m, | |
| | cyclobutyl]benzamide | 2H), 3.62 (s, 2H), 3.58-3.51 (m, 4H), | |
| | | 2.98-2.88 (m, 2H), 2.87-2.68 (m, 4H), | |
| | | 2.38-2.25 (m, 3H), 2.19-2.14 (m, 3H), | |
| | | 2.07 (s, 2H), 2.02-1.80 (m, 9H), 1.74- | |
| | | 1.65 (m, 2H), 1.51 (br t, J = 11.6 Hz, | |
| | | 2H), 1.42-1.28 (m, 2H), 1.21 (s, 6H), | |
| | | 1.14 (s, 6H) | |
| 452 | 4-[7-[4-[5-acetyl-3-[6-cyano-7- | (CDCl₃) δ = 8.50 (s, 1H), 7.66 (d, J = 8.8 | 954.8 |
| | (difluoromethyl)-3,4-dihydro- | Hz, 2H), 7.45 (d, J = 8.8 Hz, 1H), 7.34 | |
| | 2H-quinolin-1-yl]-6,7-dihydro- | (d, J = 10.4 Hz, 1H), 6.94-6.59 (m, 2H), | |
| | 4H-pyrazolo[4,3-c]pyridin-1- | 6.47 (d, J = 2.4 Hz, 1H), 6.44-6.37 (m, | |
| | yl]-1-piperidyl]-2- | 3H), 6.09 (d, J = 8.4 Hz, 1H), 4.28 (s, | |
| | azaspiro[3.5]nonan-2-yl]-N-[3- | 1H), 4.18-4.09 (m, 2H), 4.05 (s, 1H), | |
| | (4-cyano-3-methoxy-phenoxy)- | 4.00-3.86 (m, 5H), 3.80-3.68 (m, 3H), | |
| | 2,2,4,4-tetramethyl- | 3.64 (d, J = 16.8 Hz, 4H), 3.20-3.08 (m, | |
| | cyclobutyl]benzamide | 2H), 2.92-2.72 (m, 4H), 2.51-2.33 (m, | |
| | | 3H), 2.29-2.13 (m, 4H), 2.12-2.02 (m, | |
| | | 6H), 1.92-1.84 (m, 3H), 1.62-1.51 (m, | |
| | | 2H), 1.45-1.32 (m, 2H), 1.25 (d, J = 9.6 | |
| | | Hz, 12H) | |
| 455 | 4-[7-[4-[5-acetyl-3-(6-cyano-7- | δ = 7.73 (d, J = 8.4 Hz, 2H), 7.64 (d, J = | 918.7 |
| | methyl-3,4-dihydro-2H- | 8.4 Hz, 1H), 7.42 (d, J = 9.6 Hz, 1H), | |
| | quinolin-1-yl)-6,7-dihydro-4H- | 7.32 (s, 1H), 6.63 (d, J = 2.0 Hz, 1H), | |
| | pyrazolo[4,3-c]pyridin-1-yl]-1- | 6.57-6.50 (m, 1H), 6.41 (d, J = 8.4 Hz, | |
| | piperidyl]-2- | 2H), 6.36-6.29 (m, 1H), 4.26 (s, 1H), | |
| | azaspiro[3.5]nonan-2-yl]-N-[3- | 4.14-4.08 (m, 2H), 4.07-3.95 (m, 2H), | |
| | (4-cyano-3-methoxy-phenoxy)- | 3.90 (s, 3H), 3.77-3.68 (m, 2H), 3.64- | |
| | 2,2,4,4-tetramethyl- | 3.57 (m, 4H), 3.54 (s, 2H), 2.94 (br d, J = | |
| | cyclobutyl]benzamide | 7.6 Hz, 2H), 2.88-2.69 (m, 4H), 2.40- | |
| | | 2.29 (m, 3H), 2.23 (s, 3H), 2.08 (s, 2H), | |
| | | 2.00-1.82 (m, 9H), 1.70 (br d, J = 11.6 | |
| | | Hz, 2H), 1.57-1.45 (m, 2H), 1.41-1.27 | |
| | | (m, 2H), 1.21 (s, 6H), 1.14 (s, 6H) | |
| 468 | 2-[7-[4-[5-acetyl-3-[7-methyl- | δ = 8.74 (s, 2H), 8.30-8.25 (m, 1H), | 979.6 |
| | 6-(1-methylpyrazol-4-yl)-3,4- | 7.95-7.88 (m, 1H), 7.81-7.69 (m, 2H), | |
| | dihydro-2H-quinolin-1-yl]-6,7- | 7.53 (s, 1H), 7.22 (d, J = 2.4 Hz, 1H), | |
| | dihydro-4H-pyrazolo[4,3- | 7.07-6.92 (m, 2H), 6.37-6.28 (m, 1H), | |
| | c]pyridin-1-yl]-1-piperidyl]-2- | 4.30 (s, 1H), 4.16-4.02 (m, 3H), 4.01- | |
| | azaspiro[3.5]nonan-2-yl]-N-[3- | 3.91 (m, 1H), 3.89-3.80 (m, 5H), 3.78- | |
| | (3-chloro-4-cyano-phenoxy)- | 3.67 (m, 4H), 3.59-3.52 (m, 2H), 3.01- | |
| | 2,2,4,4-tetramethyl- | 2.89 (m, 2H), 2.87-2.69 (m, 4H), 2.36- | |
| | cyclobutyl]pyrimidine-5- | 2.25 (m, 3H), 2.19-2.12 (m, 3H), 2.07 | |
| | carboxamide | (s, 2H), 2.03-1.88 (m, 7H), 1.86 (br s, | |
| | | 2H), 1.76-1.64 (m, 2H), 1.57-1.46 (m, | |
| | | 2H), 1.41-1.29 (m, 2H), 1.21 (s, 6H), | |
| | | 1.11 (s, 6H) | |
| 469 | 2-[7-[4-[5-acetyl-3-[7-methyl- | δ = 8.74 (s, 2H), 8.15 (s, 1H), 7.78-7.70 | 975.9 |
| | 6-(1-methylpyrazol-4-yl)-3,4- | (m, 2H), 7.64 (d, J = 8.4 Hz, 1H), 7.52 (s, | |
| | dihydro-2H-quinolin-1-yl]-6,7- | 1H), 7.01-6.96 (m, 1H), 6.64 (d, J = 2.0 | |
| | dihydro-4H-pyrazolo[4,3- | Hz, 1H), 6.54 (m, 1H), 6.35-6.30 (m, | |
| | c]pyridin-1-yl]-1-piperidyl]-2- | 1H), 4.24 (s, 1H), 4.13-4.07 (m, 2H), | |
| | azaspiro [3.5]nonan-2-yl]-N-[3- | 4.03 (d, J = 9.2 Hz, 2H), 3.90 (s, 3H), | |
| | (4-cyano-3-methoxy-phenoxy)- | 3.84 (s, 3H), 3.82 (s, 2H), 3.75 (s, 2H), | |
| | 2,2,4,4-tetramethyl- | 3.70 (t, J = 5.6 Hz, 1H), 3.57-3.51 (m, | |
| | cyclobutyl]pyrimidine-5- | 2H), 3.04-2.94 (m, 2H), 2.82 (s, 1H), | |
| | carboxamide | 2.78-2.68 (m, 3H), 2.44-2.32 (m, 3H), | |
| | | 2.18-2.14 (m, 3H), 2.07 (s, 2H), 2.04- | |
| | | 1.82 (m, 10H), 1.77-1.68 (m, 2H), 1.52 | |
| | | (t, J = 11.6 Hz, 2H), 1.42-1.33 (m, 2H), | |
| | | 1.22 (s, 6H), 1.13 (s, 6H) | |
| 471 | 2-[7-[4-[5-acetyl-3-[7-methyl- | (CDCl₃) δ = 8.63 (s, 2H), 7.52-7.39 (m, | 959.8 |
| | 6-(1-methylpyrazol-4-yl)-3,4- | 2H), 7.34-7.24 (m, 1H), 6.98-6.84 (m, | |
| | dihydro-2H-quinolin-1-yl]-6,7- | 1H), 6.71 (d, J = 2.0 Hz, 1H), 6.65-6.55 | |
| | dihydro-4H-pyrazolo[4,3- | (m, 1H), 6.31 (s, 1H), 5.88 (br d, J = 8.0 | |
| | c]pyridin-1-yl]-1-piperidyl]-2- | Hz, 1H), 4.24-4.01 (m, 3H), 3.97 (s, | |
| | azaspiro[3.5]nonan-2-yl]-N-[3- | 1H), 3.92-3.76 (m, 9H), 3.76-3.53 (m, | |
| | (4-cyano-3-methyl-phenoxy)- | 3H), 3.21-3.01 (m, 2H), 2.81-2.62 (m, | |
| | 2,2,4,4-tetramethyl- | 4H), 2.55-2.28 (m, 7H), 2.23-2.06 (m, | |
| | cyclobutyl]pyrimidine-5- | 8H), 1.99-1.95 (m, 5H), 1.84 (br d, J = | |
| | carboxamide | 10.4 Hz, 2H), 1.53 (br t, J = 11.6 Hz, | |
| | | 2H), 1.40-1.25 (m, 2H), 1.16 (d, J = | |
| | | 10.4 Hz, 12H) | |
| 472 | 2-[7-[4-[5-acetyl-3-[7-methyl- | (CDCl₃) δ = 8.64 (s, 2H), 7.52-7.42 (m, | 973.7 |
| | 6-(1-methylpyrazol-4-yl)-3,4- | 1H), 7.37-7.26 (m, 2H), 6.98-6.82 (m, | |
| | dihydro-2H-quinolin-1-yl]-6,7- | 1H), 6.45 (d, J = 8.8 Hz, 1H), 6.35-6.23 | |
| | dihydro-4H-pyrazolo[4,3- | (m, 1H), 5.89 (d, J = 8.0 Hz, 1H), 4.23- | |
| | c]pyridin-1-yl]-1-piperidyl]-2- | 4.04 (m, 3H), 4.03-3.96 (m, 1H), 3.89- | |
| | azaspiro[3.5]nonan-2-yl]-N-[3- | 3.77 (m, 8H), 3.71-3.63 (m, 1H), 3.62- | |
| | (4-cyano-2,3-dimethyl- | 3.49 (m, 2H), 3.05 (br d, J = 11.6 Hz, | |
| | phenoxy)-2,2,4,4-tetramethyl- | 2H), 2.81-2.62 (m, 4H), 2.41 (s, 3H), | |
| | cyclobutyl]pyrimidine-5- | 2.38-2.29 (m, 3H), 2.20-2.13 (m, 9H), | |
| | carboxamide | 2.10 (s, 1H), 2.02-1.77 (m, 10H), 1.52 | |
| | | (br t, J = 12.0 Hz, 2H), 1.31 (br d, J = | |
| | | 11.6 Hz, 2H), 1.23-1.05 (m, 12H) | |
| 473 | 2-[7-[4-[5-acetyl-3-[7-methyl- | (CDCl₃) δ = 8.63 (s, 2H), 7.51-7.26 (m, | 993.8 |
| | 6-(1-methylpyrazol-4-yl)-3,4- | 3H), 7.00-6.81 (m, 1H), 6.51 (d, J = 8.8 | |
| | dihydro-2H-quinolin-1-yl]-6,7- | Hz, 1H), 6.31 (s, 1H), 5.88 (d, J = 8.0 | |
| | dihydro-4H-pyrazolo[4,3- | Hz, 1H), 4.24-3.99 (m, 4H), 3.95-3.74 | |
| | c]pyridin-1-yl]-1-piperidyl]-2- | (m, 10H), 3.71-3.53 (m, 3H), 3.03 (br d, | |
| | azaspiro[3.5]nonan-2-yl]-N-[3- | J = 10.4 Hz, 2H), 2.80-2.63 (m, 4H), | |
| | (3-chloro-4-cyano-2-methyl- | 2.40-2.24 (m, 6H), 2.23-2.06 (m, 6H), | |
| | phenoxy)-2,2,4,4-tetramethyl- | 2.04-1.95 (m, 7H), 1.81 (br d, J = 12.0 | |
| | cyclobutyl]pyrimidine-5- | Hz, 2H), 1.59-1.45 (m, 2H), 1.38-1.25 | |
| | carboxamide | (m, 2H), 1.17 (d, J = 10.4 Hz, 12H) | |

### Example 56: Synthesis of 2-[8-[[4-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]-1-piperidyl]methyl]-5-azaspiro [2.5]octan-5-yl]-N-[3-(3-chloro-4-cyano-phenoxy)-2,2,4,4-tetramethyl-cyclobutyl]pyrimidine-5-carboxamide (Compound 357)

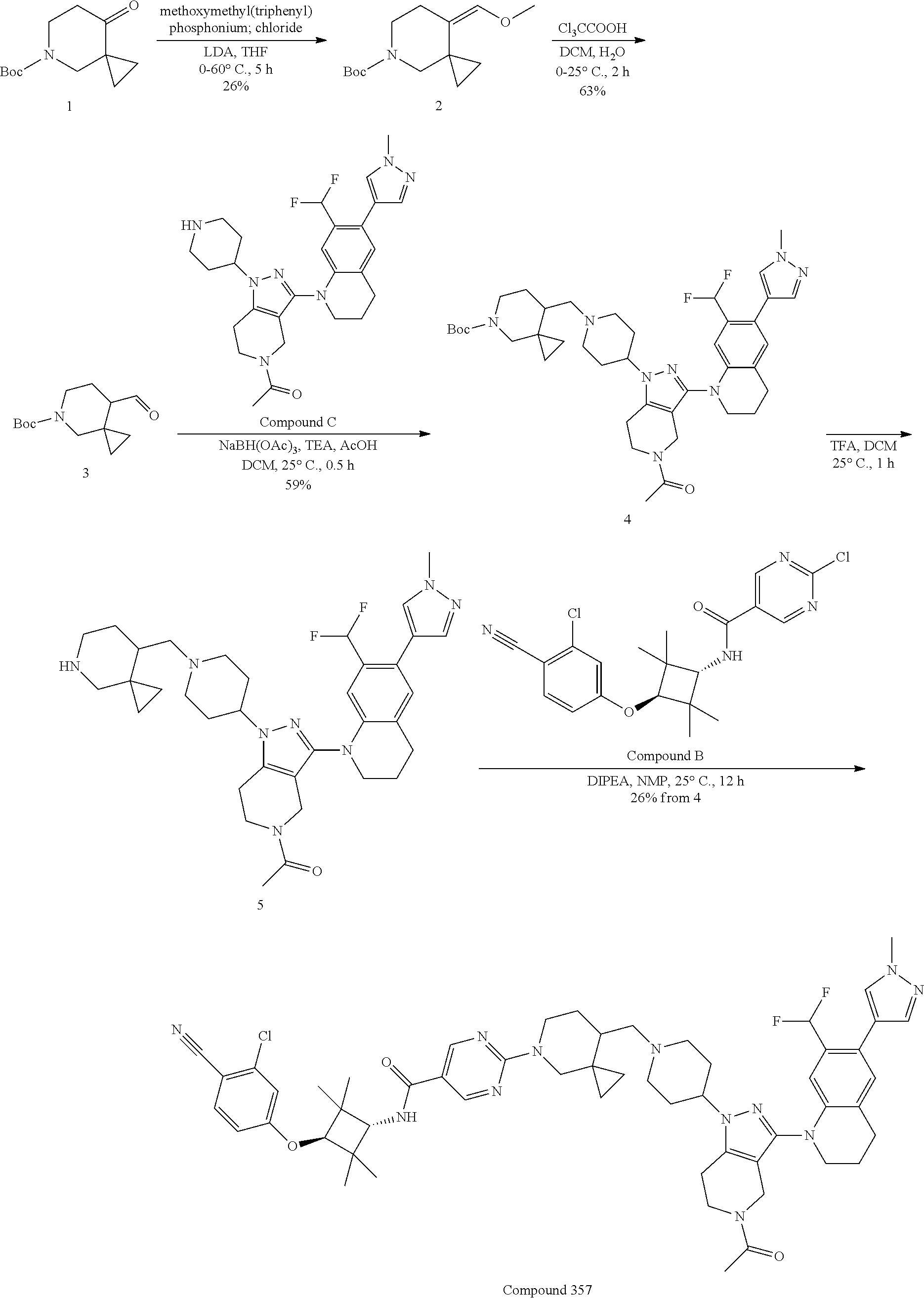

To a solution of methoxymethyl(triphenyl)phosphonium; chloride (3.04 g, 8.88 mmol, 2.0 eq) in THF (14 mL) was added dropwise LDA (2 M, 3.99 mL, 1.8 eq) under N₂ at 0° C., the mixture was stirred at 25° C. under N₂ for 2 h, then the tert-butyl 8-oxo-5-azaspiro[2.5]octane-5-carboxylate (1.00 g, 4.44 mmol, 1.0 eq) in THF (5.6 mL) was added dropwise to the reaction mixture, the mixture was stirred at 60° C. under N₂ for 3 h. The reaction mixture was quenched by addition saturated NH₄Cl aqueous solution (10 mL) at 0° C., and then extracted with EtoAc (10 mL×3). The combined organic layers were washed with water (10 mL×2), dried over anhydrous Na₂SO₄, filtered, and concentrated under reduced pressure to give a residue. The residue was purified by flash silica gel chromatography (ISCO®; 2 g SepaFlash® Silica Flash Column, Eluent of 0˜10% Ethyl acetate/Petroleum ethergradient @50 mL/min). Compound tert-butyl (8Z)-8-(methoxymethylene)-5-azaspiro[2.5]octane-5-carboxylate (300 mg, 1.18 mmol, 26% yield) was obtained as a colorless oil.

LC-MS: MS (ESI⁺): t_{R}=0.865 min, m/z=198.1 [M−55]

To a solution of tert-butyl (8Z)-8-(methoxymethylene)-5-azaspiro[2.5]octane-5-carboxylate (300 mg, 1.18 mmol, 1.0 eq) in DCM (3 mL) and H₂O (1.5 mL) was added 2,2,2-trichloroacetic acid (773 mg, 4.74 mmol, 478 μL, 4.0 eq) at 0° C. The mixture was stirred at 25° C. for 2 h. The resulting mixture was basified with saturated aqueous NaHCO₃ solution to adjust pH=8, then extracted with DCM (20 mL×3). The combined organic layer was dried over anhydrous Na₂SO₄, filtered and concentrated under reduced pressure to give a residue. The residue was purified by column chromatography (SiO₂, Petroleum ether/Ethyl acetate=10:1 to 5:1). Compound tert-butyl 8-formyl-5-azaspiro[2.5]octane-5-carboxylate (180 mg, 752 μmol, 63% yield) was obtained as a white solid.

LC-MS: MS (ESI⁺): t_{R}=0.757 min, m/z=184.0 [M−55]

To a solution of tert-butyl 8-formyl-5-azaspiro[2.5]octane-5-carboxylate (98 mg, 412 μmol, 1.5 eq) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (150 mg, 275 μmol, 1.0 eq) in DCM (1 mL) was added TEA (138 mg, 1.37 mmol, 191 μL, 5.0 eq) and NaBH(OAc)₃ (174 mg, 824 μmol, 3.0 eq) and AcOH (165 μg, 2.75 μmol, 1.57e-1 μL, 0.01 eq). The mixture was stirred at 25° C. for 0.5 h. The reaction mixture was diluted with water (2 mL) and then extracted with DCM (2 mL×3). The combined organic layer was dried over anhydrous Na₂SO₄, filtered, and concentrated under reduced pressure. The residue was purified by prep-TLC (SiO₂, DCM:MeOH=10:1). Compound tert-butyl 8-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-5-azaspiro[2.5]octane-5-carboxylate (120 mg, 163 μmol, 59% yield) was obtained as a white solid.

LC-MS: MS (ESI⁺): t_{R}=0.757 min, m/z=733.4 [M+H⁺]

To a solution of tert-butyl 8-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-5-azaspiro[2.5]octane-5-carboxylate (240 mg, 327.46 μmol, 1.0 eq) in DCM (3 mL) was added TFA (1.54 g, 13.46 mmol, 1 mL, 41.1 eq). The mixture was stirred at 25° C. for 1 h. The resulting mixture was concentrated under reduced pressure to give a residue. The crude product Compound 1-[1-[1-(5-azaspiro[2.5]octan-8-ylmethyl)-4-piperidyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (207 mg) was obtained as a white solid and directly used into the next step without further purification.

LC-MS: MS (ESI⁺): t_{R}=0.747 min, m/z=633.4 [M+H⁺]

To a solution of 1-[1-[1-(5-azaspiro[2.5]octan-8-ylmethyl)-4-piperidyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (69 mg, 109 μmol, 1.0 eq) and 2-chloro-N-[3-(3-chloro-4-cyano-phenoxy)-2,2,4,4-tetramethyl-cyclobutyl]pyrimidine-5-carboxamide (45 mg, 109 mol, 1.0 eq) in NMP (0.5 mL) was added DIPEA (28 mg, 218 μmol, 38 μL, 2.0 eq). The mixture was stirred at 25° C. for 12 h. The resulting mixture was concentrated under reduced pressure to give a residue. The residue was purified by prep-HPLC (column: Phenomenex luna C18 150*25 mm*10 um; mobile phase: [water (FA)-ACN]; gradient: 29%-59% B over 10 min). Compound 2-[8-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-5-azaspiro[2.5]octan-5-yl]-N-[3-(3-chloro-4-cyano-phenoxy)-2,2,4,4-tetramethyl-cyclobutyl]pyrimidine-5-carboxamide (27.46 mg, 29.10 μmol, 26% yield over two steps was obtained as a white solid.

¹H NMR (400 MHz, CHLOROFORM-d) δ=8.67 (s, 2H), 7.64-7.48 (m, 2H), 7.41 (d, J=6.2 Hz, 1H), 7.04 (br d, J=18.0 Hz, 1H), 6.98-6.87 (m, 2H), 6.80 (br d, J=8.8 Hz, 1H), 6.72-6.35 (m, 1H), 5.93 (br d, J=8.4 Hz, 1H), 4.38-4.18 (m, 2H), 4.18-4.10 (m, 2H), 4.04 (s, 1H), 3.96 (s, 3H), 3.91 (br t, J=5.2 Hz, 1H), 3.84-3.64 (m, 5H), 3.58-3.47 (m, 1H), 3.33-2.98 (m, 2H), 2.95-2.69 (m, 5H), 2.36-2.00 (m, 10H), 1.98-1.75 (m, 5H), 1.23 (d, J=14.0 Hz, 12H), 0.58-0.39 (m, 4H)

LC-MS: MS (ESI: t_{R}=2.119 min, m/z=1015.4 [M+H⁺]

The compounds below were prepared in a similar manner as described in Example 56.

| | | | LC-MS |
|---|---|---|---|
| Compound | Name | ¹H NMR (CDCl₃) | [M + H⁺] |
| 362 | 2-[8-[[4-[5-acetyl-3-[7- | δ = 8.67 (s, 2H), 7.53 (d, J = 5.6 Hz, 1H), | 1011.4 |
| | (difluoromethyl)-6-(1- | 7.48-7.38 (m, 2H), 7.04 (br d, J = 18.4 | |
| | methylpyrazol-4-yl)-3,4- | Hz, 1H), 6.96-6.82 (m, 1H), 6.71-6.35 | |
| | dihydro-2H-quinolin-1-yl]-6,7- | (m, 3H), 5.94 (br d, J = 7.6 Hz, 1H), 4.30 | |
| | dihydro-4H-pyrazolo[4,3- | (br d, J = 1.2 Hz, 2H), 4.12 (br d, J = 8.4 | |
| | c]pyridin-1-yl]-1- | Hz, 2H), 4.04 (s, 1H), 3.94 (brd, J = 15.2 | |
| | piperidyl]methyl]-5- | Hz, 8H), 3.79-3.65 (m, 5H), 3.59-3.51 | |
| | azaspiro[2.5] octan-5-yl]-N-[3- | (m, 1H), 3.15-3.00 (m, 1H), 2.94-2.67 | |
| | (4-cyano-3-methoxy-phenoxy)- | (m, 5H), 2.40-1.99 (m, 11H), 1.96- | |
| | 2,2,4,4-tetramethyl- | 1.75 (m, 5H), 1.24 (d, J = 6.8 Hz, 12H), | |
| | cyclobutyl]pyrimidine-5- | 0.55-0.40 (m, 4H) | |
| | carboxamide | | |

### Example 57: Synthesis of 4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutoxy]-N-[3-(4-cyano-3-methoxy-phenoxy)-2,2,4,4-tetramethyl-cyclobutyl]benzamide (Compound 375)

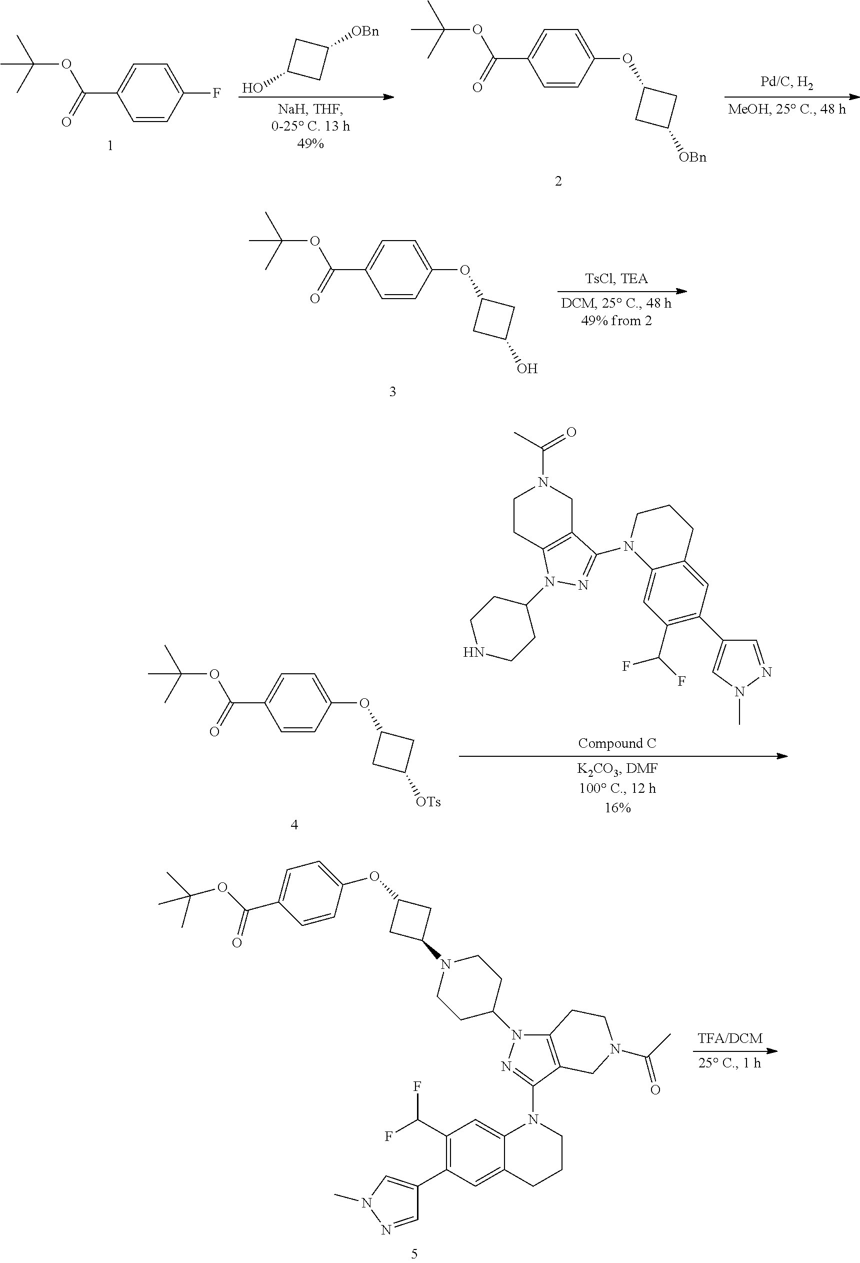

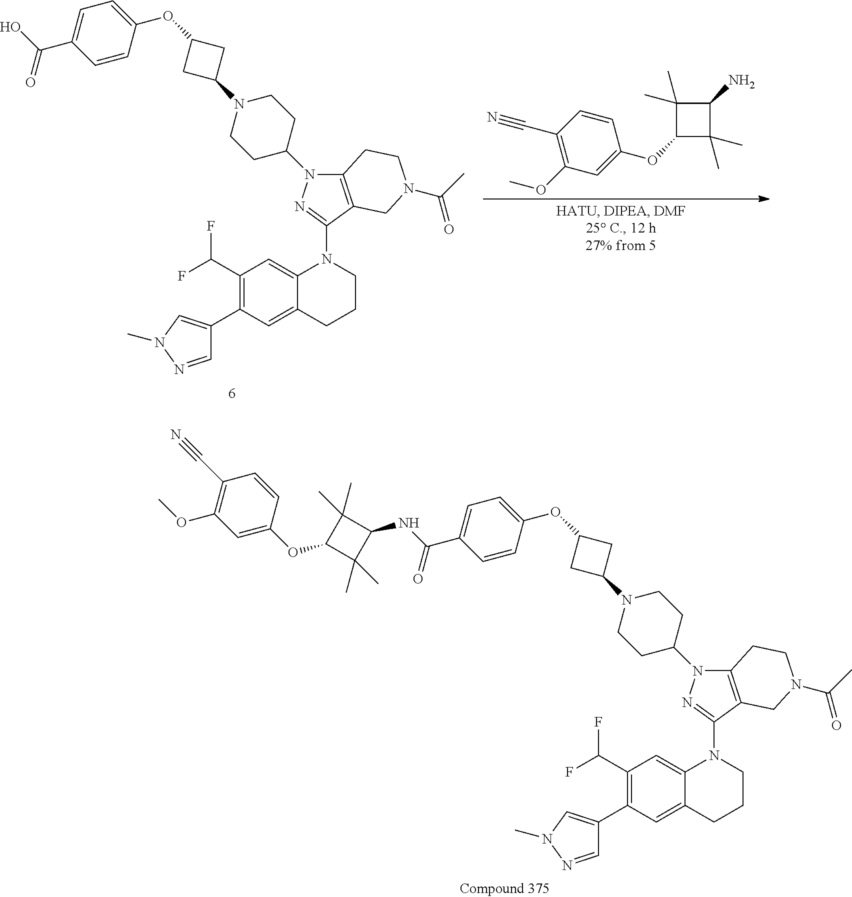

To a solution of 3-benzyloxycyclobutanol (5.00 g, 28.05 mmol, 1.0 eq) in THF (50 mL) was added NaH (1.68 g, 42.08 mmol, 94 μL, 60% purity, 1.5 eq) at 0° C. under N₂, The mixture was stirred at 0° C. for 1 h under N₂. Then tert-butyl 4-fluorobenzoate (8.26 g, 42.08 mmol, 1.5 eq) was added into it. The mixture was stirred at 25° C. for 12 h under N₂. The reaction mixture was diluted with NH₄Cl solution (50 mL) and then extracted with ethyl acetate (50 mL×3). The combined organic layer was washed with brine (20 mL), dried over anhydrous Na₂SO₄, filtered, and concentrated under reduced pressure. The residue was purified by flash silica gel chromatography (ISCO®; 10 g SepaFlash® Silica Flash Column, Eluent of 0˜20% Ethyl acetate/Petroleum ethergradient @50 mL/min). Compound tert-butyl 4-(3-benzyloxycyclobutoxy)benzoate (4.90 g, 13.82 mmol, 49% yield) was obtained as a white solid.

LC-MS: MS (ESI⁺): t_{R}=0.998 min, m/z=299.1 [M−55]

To a solution of tert-butyl 4-(3-benzyloxycyclobutoxy)benzoate (4.90 g, 13.82 mmol, 1.0 eq) in MeOH (25 mL) was added Pd/C (500 mg, 470 μmol, 10% purity, 3.40e-2 eq) under N₂ atmosphere. The suspension was degassed and purged with H₂ for 3 times. The mixture was stirred under H₂ at 25° C. for 48 h. The reaction mixture was filtered and concentrated under reduced pressure to give a residue. The crude product tert-butyl 4-(3-hydroxycyclobutoxy)benzoate (3.60 g) was obtained as a white solid and directly used in the next step without further purification.

LC-MS: MS (ESI⁺): t_{R}=0.552 min, m/z=209.1 [M−55]

To a solution of tert-butyl 4-(3-hydroxycyclobutoxy)benzoate (500 mg) in DCM (5 mL) was added TEA (765 mg, 7.57 mmol, 1.05 mL, 4.0 eq) and TsCl (721 mg, 3.78 mmol, 2.0 eq). The mixture was stirred at 25° C. for 12 h. The reaction mixture was diluted with water (50 mL) and the mixture was extracted with DCM (25 mL*3). The combined organic phase was washed with brine (30 mL), dried over anhydrous Na₂SO₄, filtered, and concentrated in vacuo. The residue was purified by column chromatography (SiO₂, Petroleum ether: Ethyl acetate=10:1 to 5:1). Compound tert-butyl 4-[3-(p-tolylsulfonyloxy)cyclobutoxy]benzoate (400 mg, 955.79 μmol, 49% yield over two steps) was obtained as a white solid.

LC-MS: MS (ESI⁺): t_{R}=0.720 min, m/z=441.1 [M+Na⁺]

To a solution of tert-butyl 4-[3-(p-tolylsulfonyloxy)cyclobutoxy]benzoate (201 mg, 481 μmol, 2.0 eq) in DMF (2 mL) was added K₂CO₃ (99 mg, 722 μmol, 3.0 eq) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (150 mg, 240 μmol, 1.0 eq). The mixture was stirred at 100° C. for 12 h. The resulting mixture was concentrated under reduced pressure to give a residue. The residue was purified by prep-HPLC (column: Phenomenex luna C18 150*25 mm*10 um; mobile phase: [water(FA)-ACN]; gradient: 30%-60% B over 10 min). Compound tert-butyl 4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutoxy]benzoate (30 mg, 39.69 μmol, 16% yield) was obtained as a white solid.

LC-MS: MS (ESI⁺): t_{R}=0.567 min, m/z=756.4 [M+H⁺]

To a solution of tert-butyl 4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutoxy]benzoate (20 mg, 26 μmol, 1.0 eq) in DCM (1 mL) was added TFA (767 mg, 6.73 mmol, 0.5 mL, 254.4 eq). The mixture was stirred at 25° C. for 1 h. The reaction mixture was concentrated under reduced pressure to give a residue. The crude product 4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutoxy]benzoic acid (21 mg) was obtained as a white solid and directly used in the next step without further purification.

LC-MS: MS (ESI⁺): t_{R}=0.719 min, m/z=700.3 [M+H⁺]

To a solution of 4-(3-amino-2,2,4,4-tetramethyl-cyclobutoxy)-2-methoxy-benzonitrile (10 mg, 26 μmol, 1.0 eq) and 4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]cyclobutoxy]benzoic acid (21 mg) in DMF (0.5 mL) was added DIPEA (6 mg, 52 μmol, 9 μL, 2.0 eq) and HATU (12 mg, 34 μmol, 1.3 eq). The mixture was stirred at 25° C. for 12 h. The reaction mixture was concentrated under reduced pressure to give a residue. The residue was purified by prep-HPLC (column: Phenomenex luna C18 150*25 mm*10 um; mobile phase: [water (FA)-ACN]; gradient: 30%-60% B over 10 mi). Compound 4-[3-[4-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]-1-piperidyl]cyclobutoxy]-N-[3-(4-cyano-3-methoxy-phenoxy)-2,2,4, 4-tetramethyl-cyclobutyl]benzamide (6.88 mg, 7.09 μmol, 27 yield over two steps) was obtained as a white solid.

¹H NMR (400 MHz, CHLOROFORM-d) δ=7.66 (d, J=8.8 Hz, 2H), 7.48-7.31 (m, 3H), 7.04-6.92 (m, 2H), 6.71 (d, J=8.8 Hz, 2H), 6.51-6.05 (m, 4H), 4.90 (brs, 1H), 4.51-4.28 (m, 2H), 4.15 (s, 1H), 4.07 (d, J=8.0 Hz, 1H), 3.99 (s, 1H), 3.91-3.80 (m, 7H), 3.73-3.53 (m, 5H), 3.38 (br s, 2H), 2.95-2.46 (m, 10H), 2.19-1.91 (m, 7H), 1.20 (s, 6H), 1.17 (s, 6H)

LC-MS: MS (ESI⁺): t_{R}=3.027 min, m/z=956.7 [M+H⁺]

The compounds below were prepared in a similar manner as described in Example 57.

| | | | LC-MS |
|---|---|---|---|
| Compound | Name | ¹H NMR (CDCl₃) | [M + H⁺] |
| 376 | 2-[3-[4-[5-acetyl-3-[7- | δ = 8.83 (s, 2H), 8.37 (s, 1H), 7.57-7.43 | 962.7 |
| | (difluoromethyl)-6-(1- | (m, 2H), 7.35 (d, J = 5.1 Hz, 1H), 7.03- | |
| | methylpyrazol-4-yl)-3,4- | 6.70 (m, 4H), 6.66-6.27 (m, 1H), 6.17- | |
| | dihydro-2H-quinolin-1-yl]-6,7- | 6.01 (m, 1H), 5.37-5.22 (m, 1H), 4.20 | |
| | dihydro-4H-pyrazolo[4,3- | (s, 1H), 4.12-3.95 (m, 4H), 3.93-3.79 | |
| | c]pyridin-1-yl]-1- | (m, 4H), 3.76-3.54 (m, 3H), 3.38-3.00 | |
| | piperidyl]cyclobutoxy]-N-[3-(3- | (m, 3H), 2.87-2.71 (m, 3H), 2.70-2.50 | |
| | chloro-4-cyano-phenoxy)- | (m, 3H), 2.42-2.32 (m, 2H), 2.26-2.13 | |
| | 2,2,4,4-tetramethyl- | (m, 3H), 2.10 (s, 2H), 2.05-1.94 (m, | |
| | cyclobutyl]pyrimidine-5- | 6H), 1.24-1.14 (m, 12H) | |
| | carboxamide | | |
| 381 | 4-[4-[4-[5-acetyl-3-[7- | (DMSO-d₆): δ = 7.81 (d, J = 8.4 Hz, 2H), | 984.8 |
| | (difluoromethyl)-6-(1- | 7.76 (s, 1H), 7.70-7.63 (m, 2H), 7.54- | |
| | methylpyrazol-4-yl)-3,4- | 7.49 (m, 1H), 7.14-6.89 (m, 4H), 6.82- | |
| | dihydro-2H-quinolin-1-yl]-6,7- | 6.53 (m, 3H), 4.42-4.34 (m, 1H), 4.30- | |
| | dihydro-4H-pyrazolo[4,3- | 4.26 (m, 1H), 4.20-4.11 (m, 2H), 4.06 | |
| | c]pyridin-1-yl]-1- | (d, J = 9.6 Hz, 1H), 4.03-3.97 (m, 1H), | |
| | piperidyl]cyclohexoxy]-N-[3- | 3.91 (s, 3H), 3.87 (s, 3H), 3.76-3.68 (m, | |
| | (4-cyano-3-methoxy-phenoxy)- | 2H), 3.61-3.57 (m, 2H), 3.02-2.80 (m, | |
| | 2,2,4,4-tetramethyl- | 5H), 2.80-2.68 (m, 1H), 2.46-2.29 (m, | |
| | cyclobutyl]benzamide | 3H), 2.16-2.05 (m, 4H), 2.03-1.91 (m, | |
| | | 5H), 1.91-1.78 (m, 4H), 1.52-1.35 (m, | |
| | | 4H), 1.23 (s, 6H), 1.15 (s, 6H) | |
| 382 | 2-[4-[4-[5-acetyl-3-[7- | (DMSO-d₆): δ = 8.96 (s, 2H), 8.03 (d, J = | 990.8 |
| | (difluoromethyl)-6-(1- | 9.2 Hz, 1H), 7.90 (d, J = 8.8 Hz, 1H), | |
| | methylpyrazol-4-yl)-3,4- | 7.75 (s, 1H), 7.50 (s, 1H), 7.22 (d, J = 2.0 | |
| | dihydro-2H-quinolin-1-yl]-6,7- | Hz, 1H), 7.10 (s, 1H), 7.04-6.98 (m, | |
| | dihydro-4H-pyrazolo[4,3-c] | 1H), 6.96-6.63 (m, 2H), 5.02-4.89 (m, | |
| | pyridin-1-yl]-1-piperidyl] | 1H), 4.30 (s, 1H), 4.19-4.10 (m, 2H), | |
| | cyclohexoxy]-N-[3-(3-chloro-4- | 4.06 (br d, J = 9.2 Hz, 2H), 3.86 (s, 3H), | |
| | cyano-phenoxy)-2,2,4,4- | 3.76-3.67 (m, 2H), 3.62-3.56 (m, 2H), | |
| | tetramethyl-cyclobutyl] | 3.08-3.00 (m, 2H), 2.92-2.68 (m, 5H), | |
| | pyrimidine-5-carboxamide | 2.61-2.55 (m, 1H), 2.17 (br s, 2H), 2.09- | |
| | | 1.82 (m, 12H), 1.50 (br s, 4H), 1.23 (s, | |
| | | 6H), 1.12 (s, 6H) | |

### Example 58: Synthesis of 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-2-azaspiro[3.5]nonan-2-yl]-N-[3-(4-cyano-3-methoxy-phenoxy)-2,2,4,4-tetramethyl-cyclobutyl]benzamide (Compound 378)

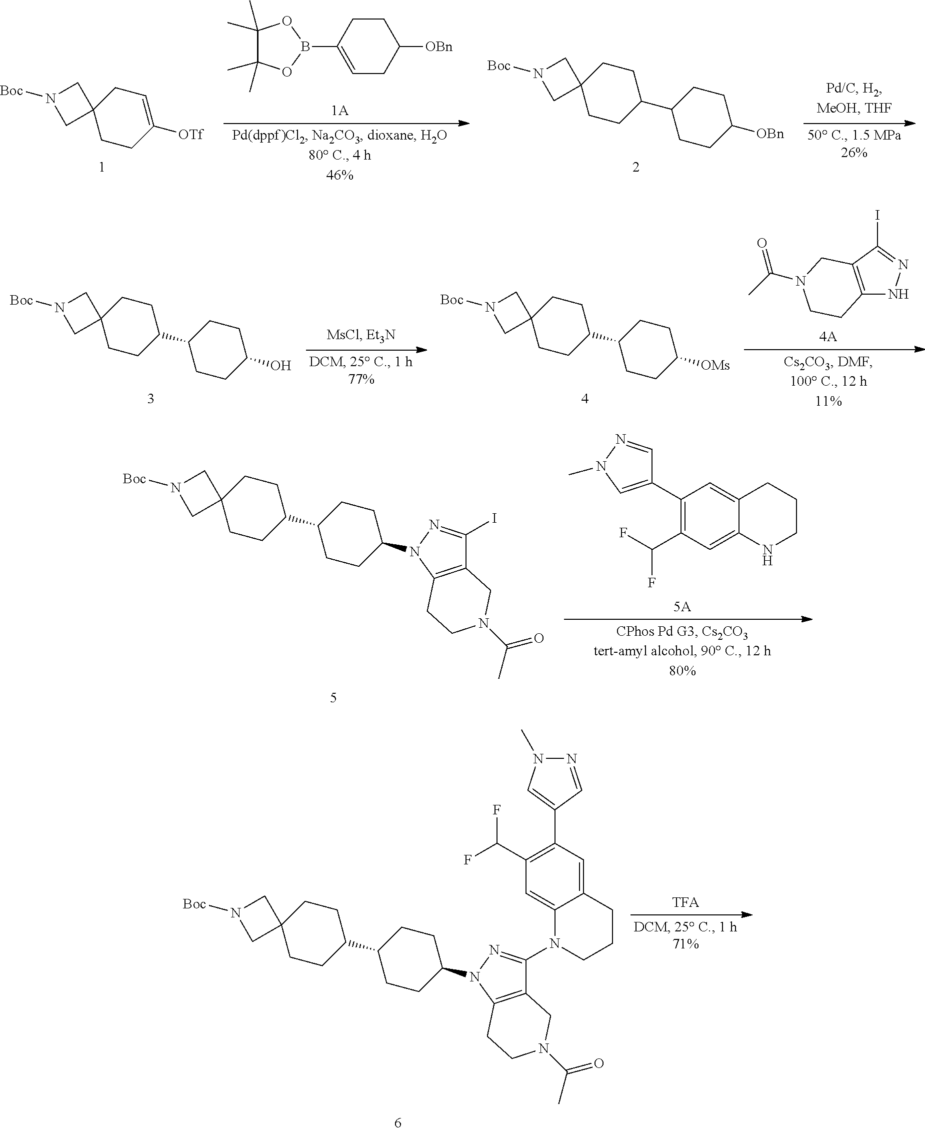

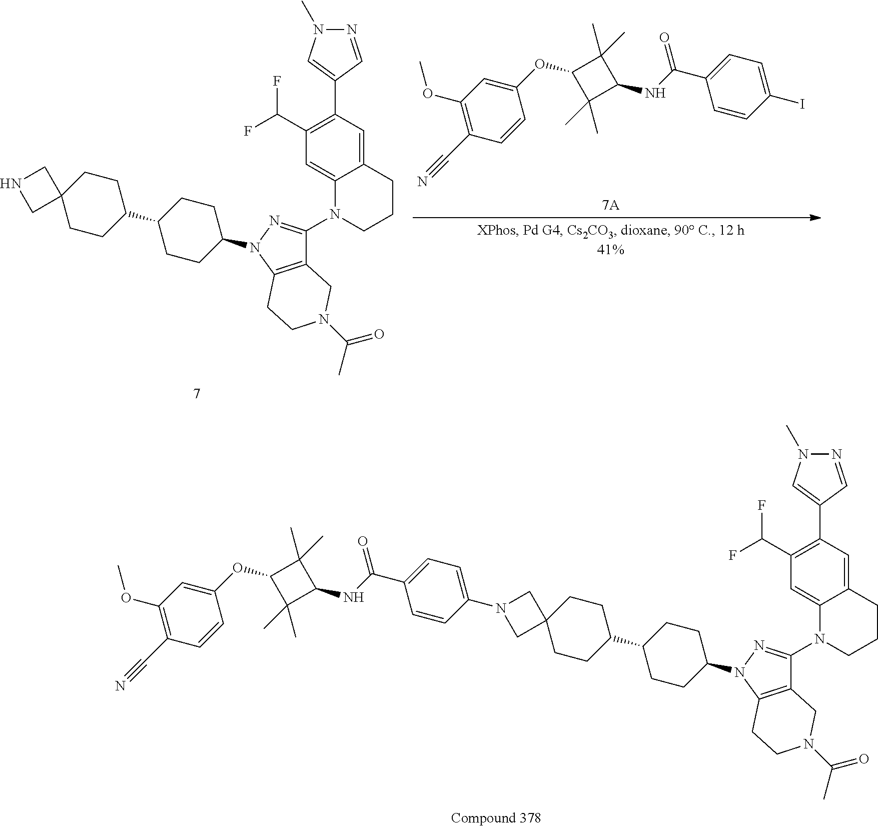

Synthesis of 1 was reported in WO2021/216661 A1.

Synthesis of 1A was reported in WO2019/236487 A1.

A mixture of 2-(4-benzyloxycyclohexen-1-yl)-4,4,5,5-tetramethyl-1,3,2-dioxaborolane (7.11 g, 22.6 mmol, 1.2 eq), tert-butyl 7-(trifluoromethylsulfonyloxy)-2-azaspiro[3.5]non-6-ene-2-carboxylate (7.00 g, 18.8 mmol, 1.0 eq), Na₂CO₃ (5.99 g, 56.5 mmol, 3.0 eq) and Pd(dppf)Cl₂ (1.38 g, 1.88 mmol, 0.1 eq) in dioxane (200 mL) and H₂O (20 mL) was degassed and purged with N₂ for 3 times, and then the mixture was stirred at 80° C. for 4 h under N₂ atmosphere. The mixture was diluted with water (200 mL) then extracted with EtOAc (50 mL×3). The combined organic phase was washed with brine (30 mL×2), dried with anhydrous Na₂SO₄, filtered and concentrated in vacuum. The residue was purified by silica gel chromatography (Petroleum ether/Ethyl acetate=1/0 to 20/1). Compound tert-butyl 7-(4-benzyloxycyclohexen-1-yl)-2-azaspiro [3.5]non-6-ene-2-carboxylate (3.60 g, 8.79 mmol, 46% yield) was obtained as a yellow oil.

LC-MS: MS (ESI⁺): t_{R}=0.681 min, m/z=354.1 [M-tBu⁺]

Solution 1: {tert-butyl 7-(4-benzyloxycyclohexen-1-yl)-2-azaspiro [3.5]non-6-ene-2-carboxylate (3.60 g, 8.79 mmol, 1.0 eq) in MeOH (20 mL) and THF (20 mL). The fixed bed (named FLR1, volume 5 mL) was packed with granular catalyst 5% Pd/C (WXC1030, 3.00 g). The H₂ back pressure regulator was adjusted to 1.5 MPa and the flow rate of H₂ was 20 mL/min. Then the solution S1 was pumped by Pump 1{S1, P1,0.303 mL/min} to fixed bed{FLRT, SS, Fixed bed, 6.350(1/4″) mm, 1 mL, 50° C.}. Then the reaction mixture was collected from the reactor output. After the reaction fluid injection completed, the fixed bed was washed by extra MeOH (20 mL). The residue was purified by silica gel chromatography (Petroleum ether/Ethyl acetate=4/1 to 3/1). Compound tert-butyl 7-(4-hydroxycyclohexyl)-2-azaspiro [3.5]nonane-2-carboxylate (750 mg, 2.32 mmol, 26% yield) was obtained as a white solid.

To a solution of tert-butyl 7-(4-hydroxycyclohexyl)-2-azaspiro [3.5]nonane-2-carboxylate (1.20 g, 3.71 mmol, 1.0 eq) in DCM (15 mL) was added Et₃N (1.13 g, 11.13 mmol, 1.55 mL, 3.0 eq) and methylsulfonyl methanesulfonate (1.29 g, 7.42 mmol, 2.0 eq). The mixture was stirred at 25° C. for 1 h. The reaction mixture was quenched by addition water 60 mL at 25° C., and then extracted with DCM (30 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/0 to 10/1). Compound tert-butyl 7-(4-methylsulfonyloxycyclohexyl)-2-azaspiro [3.5]nonane-2-carboxylate (1.16 g, 2.89 mmol, 77% yield) was obtained as a colorless oil.

¹H NMR (400 MHz, CDCl₃) δ=4.97 (s, 1H), 3.56 (d, J=14.4 Hz, 4H), 3.01 (s, 3H), 2.08 (d, J=12.0 Hz, 2H), 1.90 (d, J=13.2 Hz, 2H), 1.68 (d, J=12.0 Hz, 2H), 1.59-1.53 (m, 4H), 1.44 (s, 9H), 1.43-1.34 (m, 4H), 1.15-0.91 (m, 4H).

To a solution of tert-butyl 7-(4-methylsulfonyloxycyclohexyl)-2-azaspiro[3.5]nonane-2-carboxylate (1.06 g, 2.64 mmol, 1.0 eq) in DMF (10 mL) was added Cs₂CO₃ (2.58 g, 7.92 mmol, 3.0 eq) and 1-(3-iodo-1,4,6,7-tetrahydropyrazolo[4,3-c]pyridin-5-yl)ethanone (768 mg, 2.64 mmol, 1.0 eq). The mixture was stirred at 100° C. for 12 h. The reaction mixture was quenched by addition water (100 mL) at 25° C., and then extracted with EtOAc (50 mL×3). The combined organic layers were washed with brine (70 mL×2), dried over Na₂SO₄, filtered and concentrated under reduced pressure to give a residue. The residue was purified by prep-HPLC (column: Phenomenex luna C18 250*50 mm*10 um; mobile phase: [water(TFA)-ACN]; gradient: 55%-85% B over 21 min) and prep-HPLC (column: Welch Ultimate XB—CN 250*50*10 um; mobile phase: [Hexane-EtOH]; gradient: 1%-40% B over 15 min). Compound tert-butyl 7-[4-(5-acetyl-3-iodo-6,7-dihydro-4H-pyrazolo[4,3-c]pyridin-1-yl)cyclohexyl]-2-azaspiro[3.5]nonane-2-carboxylate (190 mg, 318 mol, 11% yield) was obtained as a white solid.

¹H NMR (400 MHz, CDCl₃) δ=4.45-4.20 (m, 2H), 3.92-3.67 (m, 3H), 3.56 (d, J=16.8 Hz, 4H), 2.78-2.62 (m, 2H), 2.24-2.14 (m, 3H), 1.90 (d, J=12.4 Hz, 8H), 1.65 (d, J=13.6 Hz, 2H), 1.45 (s, 9H), 1.43-1.33 (m, 2H), 1.23-0.93 (m, 6H).

A mixture of tert-butyl 7-[4-(5-acetyl-3-iodo-6,7-dihydro-4H-pyrazolo[4,3-c]pyridin-1-yl)cyclohexyl]-2-azaspiro[3.5]nonane-2-carboxylate (180 mg, 301 μmol, 1.0 eq), 7-(difluoromethyl)-6-(1-methylpyrazol-4-yl)-1,2,3,4-tetrahydroquinoline (95 mg, 362 μmol, 1.2 eq), CPhos Pd G3 (24 mg, 30 μmol, 0.1 eq), Cs₂CO₃ (295 mg, 905 μmol, 3.0 eq) in 2-methylbutan-2-ol (5 mL) was degassed and purged with N₂ for 3 times, and then the mixture was stirred at 90° C. for 12 h under N₂ atmosphere. The reaction mixture was quenched by addition water (60 mL) at 25° C., and then extracted with EtOAc (50 mL×3). The combined organic layers were washed with brine (70 mL×2), dried over Na₂SO₄, filtered and concentrated under reduced pressure to give a residue. The residue was purified by prep-TLC (SiO₂, DCM/MeOH=15/1). Compound tert-butyl 7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-2-azaspiro[3.5]nonane-2-carboxylate (178 mg, 243 μmol, 80% yield) was obtained as a yellow solid.

LC-MS: MS (ESI⁺): t_{R}=0.651 min, m/z=732.4 [M+H⁺]

To a solution of tert-butyl 7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-2-azaspiro[3.5]nonane-2-carboxylate (178 mg, 243 μmol, 1.0 eq) in DCM (2 mL) was added TFA (2 mL). The mixture was stirred at 25° C. for 1 h. The reaction mixture was concentrated under reduced pressure to give a residue. The residue was purified by prep-HPLC (column: Phenomenex Luna C18 150*25 mm*10 um; mobile phase: [H₂O (0.225% FA)-ACN]; gradient: 18%-48% B over 15.0 min). Compound 1-[1-[4-(2-azaspiro[3.5]nonan-7-yl)cyclohexyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (110 mg, 174 μmol, 71% yield) was obtained as a white solid.

LC-MS: MS (ESI⁺): t_{R}=0.442 min, m/z=632.5 [M+H⁺]

A mixture of 1-[1-[4-(2-azaspiro[3.5]nonan-7-yl)cyclohexyl]-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (100 mg, 158 μmol, 1.0 eq), N-[3-(4-cyano-3-methoxy-phenoxy)-2,2,4,4-tetramethyl-cyclobutyl]-4-iodo-benzamide (87 mg, 174 μmol, 1.1 eq), Xphos Pd G4 (13 mg, 15 μmol, 0.1 eq), Cs₂CO₃ (154 mg, 474 μmol, 3.0 eq) in dioxane (8 mL) was degassed and purged with N₂ for 3 times, and then the mixture was stirred at 90° C. for 12 h under N₂ atmosphere. The reaction mixture was quenched by addition water (40 mL) at 25° C., and then extracted with EtOAc (20 mL×3). The combined organic layers were washed with brine (50 mL×2), dried over Na₂SO₄, filtered and concentrated under reduced pressure to give a residue. The residue was purified by prep-HPLC (column: Phenomenex Luna C18 150*25 mm*10 um; mobile phase: [H₂O (0.225% FA)-ACN]; gradient: 80%-100% B over 9.0 min). Compound 4-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]cyclohexyl]-2-azaspiro[3.5]nonan-2-yl]-N-[3-(4-cyano-3-methoxy-phenoxy)-2,2,4,4-tetramethyl-cyclobutyl]benzamide (66.16 mg, 65.62 μmol, 41% yield) was obtained as a white solid.

¹H NMR (400 MHz, DMSO-d₆) δ=7.76-7.70 (m, 3H), 7.65 (d, J=8.4 Hz, 1H), 7.50 (s, 1H), 7.44 (d, J=9.2 Hz, 1H), 7.09 (s, 1H), 6.81-6.61 (m, 3H), 6.53 (m, 1H), 6.41 (d, J=8.8 Hz, 2H), 4.26 (s, 1H), 4.18-4.10 (m, 2H), 4.04 (d, J=9.6 Hz, 1H), 3.97 (d, J=4.4 Hz, 1H), 3.90 (s, 3H), 3.86 (s, 3H), 3.75-3.66 (m, 2H), 3.61-3.52 (m, 6H), 3.36 (s, 5H), 2.86-2.81 (m, 3H), 2.75-2.69 (m, 1H), 2.07 (s, 2H), 1.96 (s, 3H), 1.92 (d, J=12.4 Hz, 4H), 1.85-1.73 (m, 4H), 1.66 (m, 2H), 1.52-1.42 (m, 2H), 1.21 (s, 6H), 1.14 (s, 7H).

LC-MS: MS (ESI⁺): t_{R}=3.661 min, m/z=1008.8 [M+H⁺]

The compounds below were prepared in a similar manner as described in Example 58.

| | | | LC-MS |
|---|---|---|---|
| Compound | Name | ¹H NMR (CDCl₃) | [M + H⁺] |
| 377 | 2-[7-[4-[5-acetyl-3-[7- | δ = 8.72 (s, 2 H), 7.60-7.58 (d, J = 8.8 Hz, | 1014.7 |
| | (difluoromethyl)-6-(1- | 1 H), 7.56-7.54 (d, J = 5.6 Hz, 1 H), 7.42- | |
| | methylpyrazol-4-yl)-3,4- | 7.41 (m, 1 H), 7.06 (s, 1 H), 6.98-6.97 | |
| | dihydro-2H-quinolin-1-yl]-6,7- | (m, 1 H), 6.88 (s, 1 H), 6.83-6.81 (d, | |
| | dihydro-4H-pyrazolo[4,3- | J = 10.8 Hz, 1 H), 6.54-6.41 (m, 1 H), | |
| | c]pyridin-1-yl]cyclohexyl]-2- | 5.94-5.92 (d, J = 7.2 Hz, 1 H), 4.80 (s, | |
| | azaspiro[3.5]nonan-2-yl]-N-[3- | 1H), 4.27 (s, 1H), 4.14-4.13 (m, 2H), | |
| | (3-chloro-4-cyano-phenoxy)- | 4.12 (s, 1H), 4.06 (s, 3H), 3.91-3.90 (m, | |
| | 2,2,4,4-tetramethyl- | 6 H), 3.85-3.73 (m, 3H), 2.91-2.90 (m, | |
| | cyclobutyl]pyrimidine-5- | 2H), 2.75-2.74 (m, 1 H), 2.74-2.73 | |
| | carboxamide | (m, 1 H), 2.18(s, 1H), 2.09-2.08 (m, 4H), | |
| | | 2.03-2.02(m, 5H), 2.01-1.95 (m, 3 H), | |
| | | 1.77-1.74 (m, 2 H), 1.60-1.55 (m, 2 H), | |
| | | 1.26-1.23 (m, 12 H), 1. 14-1.09 (m, 5 H) | |
| 379 | 2-[7-[4-[5-acetyl-3-[7- | δ = 8.70 (s, 2H), 7.57 (d, J = 8.8 Hz, 1H), | 1014.7 |
| | (difluoromethyl)-6-(1- | 7.54 (d, J = 5.2 Hz, 1H), 7.41 (d, J = 6.4 | |
| | methylpyrazol-4-yl)-3,4- | Hz, 1H), 7.06-6.99 (m, 1H), 6.97 (d, J = | |
| | dihydro-2H-quinolin-1-yl]-6,7- | 2.4 Hz, 1H), 6.92 (d, J = 9.6 Hz, 1H), | |
| | dihydro-4H-pyrazolo[4,3- | 6.81 (dd, J = 2.4, 8.8 Hz, 1H), 6.69-6.36 | |
| | c]pyridin-1-yl]cyclohexyl]-2- | (m, 1H), 5.92 (d, J = 8.0 Hz, 1H), 4.31- | |
| | azaspiro[3.5]nonan-2-yl]-N-[3- | 4.13 (m, 2H), 4.12 (d, J = 8.4 Hz, 1H), | |
| | (3-chloro-4-cyano-phenoxy)- | 4.04 (s, 1H), 4.00 (td, J = 4.4, 9.6 Hz, | |
| | 2,2,4,4-tetramethyl- | 1H), 3.96 (d, J = 2.0 Hz, 3H), 3.92-3.88 | |
| | cyclobutyl]pyrimidine-5- | (m, 1H), 3.85 (d, J = 14.0 Hz, 4H), 3.80- | |
| | carboxamide | 3.64 (m, 3H), 2.92-2.84 (m, 2H), 2.82- | |
| | | 2.70 (m, 2H), 2.19-2.03 (m, 7H), 2.02- | |
| | | 1.94 (m, 4H), 1.87 (d, J = 12.0 Hz, 2H), | |
| | | 1.75 (dd, J = 1.6, 9.6 Hz, 2H), 1.59- | |
| | | 1.45 (m, 5H), 1.33-1.29 (m, 1H), 1.23 | |
| | | (d, J = 12.4 Hz, 12H), 1.01-0.88 (m, | |
| | | 2H) | |
| 380 | 4-[7-[4-[5-acetyl-3-[7- | (DMSO-d₆) δ = 7.78-7.65 (m, 3H), 7.64- | 1008.7 |
| | (difluoromethyl)-6-(1- | 7.60 (m, 1H), 7.49 (s, 1H), 7.40-7.35 | |
| | methylpyrazol-4-yl)-3,4- | (m, 1H), 7.09 (s, 1H), 6.95-6.85 (m, | |
| | dihydro-2H-quinolin-1-yl]-6,7- | 2H), 6.63 (s, 1H), 6.54-6.51 (m, 2H), | |
| | dihydro-4Hpyrazolo[4,3- | 6.45-6.35 (m, 2H), 4.26 (s, 1H), 4.24- | |
| | c]pyridin-1-yl]cyclohexyl]-2- | 4.16 (m, 3H), 4.09-4.03 (m, 1H), 3.87 | |
| | azaspiro[3.5]nonan-2-yl]-N-[3- | (d, J = 2.0 Hz, 6H), 3.75-3.63 (m, 2H), | |
| | (4-cyano-3-methoxy-phenoxy)- | 3.59-3.45 (m, 6H), 2.85-2.61 (m, 4H), | |
| | 2,2,4,4-tetramethyl- | 2.07 (s, 2H), 2.02-1.89 (m, 9H), 1.88- | |
| | cyclobutyl]benzamide | 1.62 (m, 4H), 1.55-1.42 (m, 4H), 1.39- | |
| | | 1.36 (m, 1H), 1.35-1.12 (m, 13H), 0.95- | |
| | | 0.85 (m, 2H) | |

### Example 59: Synthesis of 2-[7-[4-[5-acetyl-3-[7-(difluoromethyl)-6-(1-methyl pyrazol-4-yl)-3,4-dihydro-2H-quinolin-1-yl]-6,7-dihydro-4H-pyrazolo[4,3-c]pyridin-1-yl]-1-piperidyl]spiro[3.5]nonan-2-yl]oxy-N-[3-(3-chloro-4-cyano-phenoxy)-2,2,4,4-tetramethyl-cyclobutyl]pyrimidine-5-carboxamide (Compound 383)

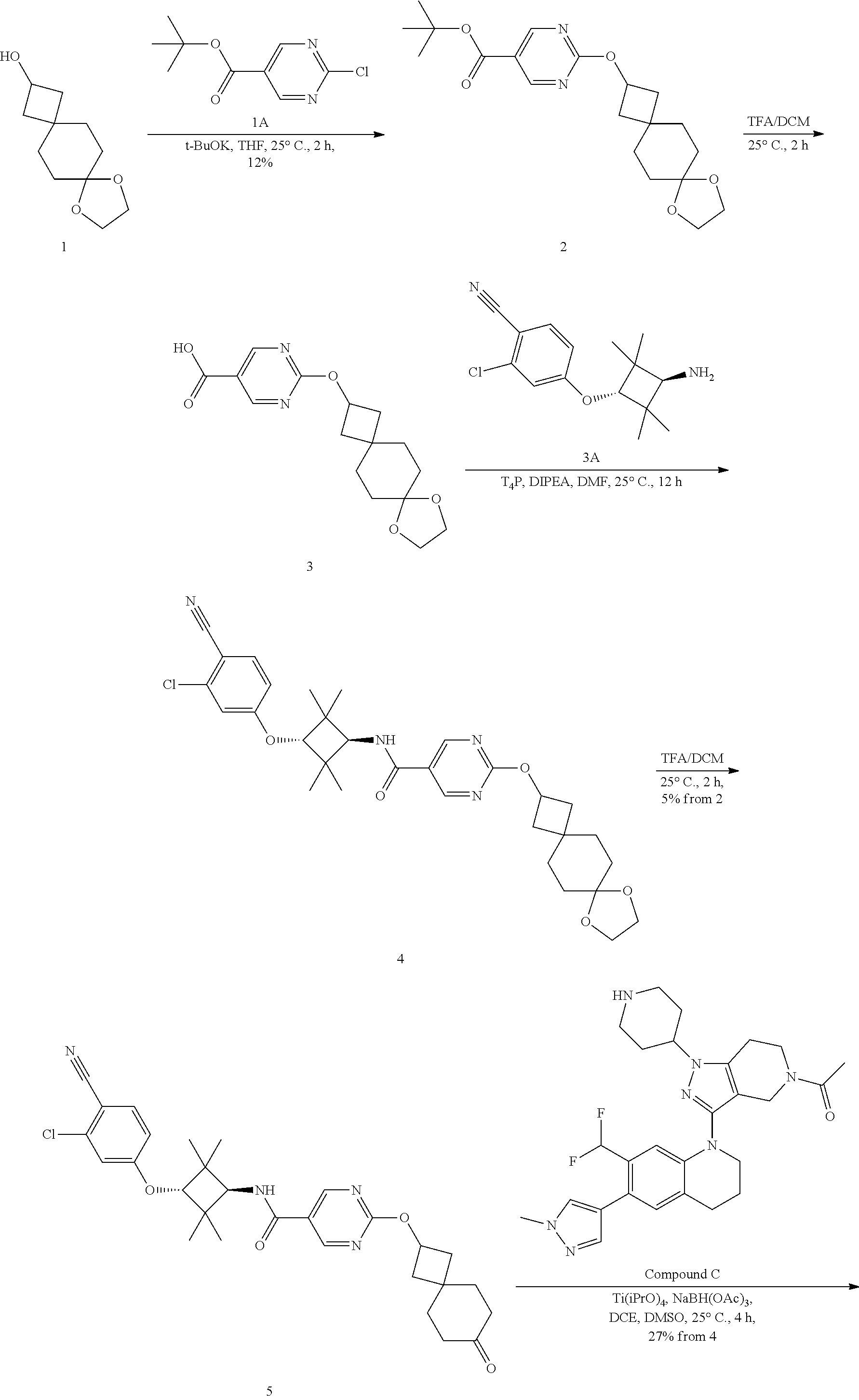

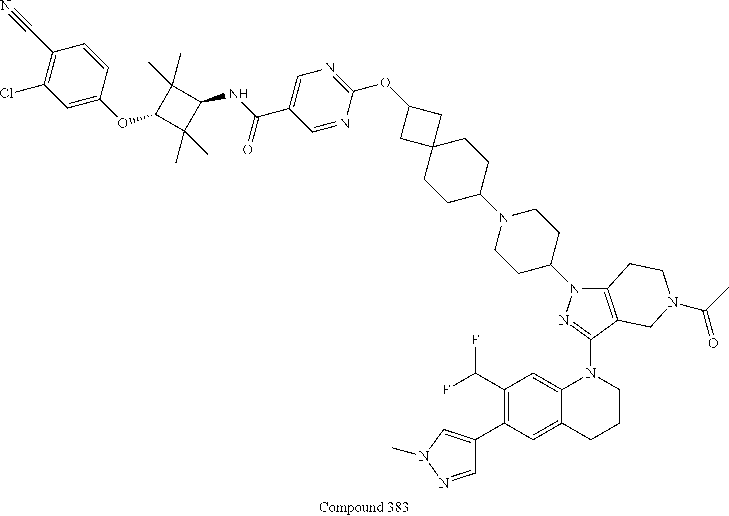 [SMILES: CC(=O)N1CCc2c(c(N3CCCc4cc(-c5cnn(C)c5)c(C(F)F)cc43)nn2C2CCN(C3CCC4(CC3)CC(Oc3ncc(C(=O)N[C@H]5C(C)(C)[C@H](Oc6ccc(C#N)c(Cl)c6)C5(C)C)cn3)C4)CC2)C1]

Synthesis of 1 was reported in WO2022/122876 A1.

Synthesis of 1A was reported in U.S. Ser. No. 10/836,749 B1.

Synthesis of 3A was reported in US2017/8904 A1.

To a solution of tert-butyl 2-chloropyrimidine-5-carboxylate (220 mg, 1.02 mmol, 1.0 eq) 8,11-dioxadispiro[3.2.47.24]tridecan-2-ol (200 mg, 1.01 mmol, 1.0 eq) was added t-BuOK (1 M, 2 mL, 2.0 eq) at 25° C. The mixture was stirred at 60° C. for 2 h. The reaction mixture was concentrated under reduced pressure to give a residue. The residue was purified by column chromatography (SiO₂, Petroleum ether/Ethyl acetate=3/1 to 0/1). Compound tert-butyl 2-(8,11-dioxadispiro[3.2.47.24]tridecan-2-yloxy) pyrimidine-5-carboxylate (45 mg, 120 mol, 12% yield) was obtained as white solid.

LC-MS: MS (ESI⁺): t_{R}=0.645 min, m/z=377.2 [M+H⁺]

To a solution of tert-butyl 2-(8,11-dioxadispiro[3.2.47.24]tridecan-2-yloxy) pyrimidine-5-carboxylate (45 mg, 120 mol, 1.0 eq) in DCM (0.5 mL) was added TFA (0.5 mL) at 25° C. The mixture was stirred at 25° C. for 2 h. Then to the residue was added dichloromethane/Methanol (10/1) 50 mL, and adjusted the pH to 7-8 with ammonium hydroxide, dried over anhydrous sodium sulfate, filtered and concentrated in vacuum to give a residue. Compound 2-(8,11-dioxadispiro[3.2.47.24]tridecan-2-yloxy) pyrimidine-5-carboxylic acid (38 mg) was obtained as a yellow solid and directly used in the next step without further purification.

LC-MS: MS (ESI⁺): t_{R}=0.483 min, m/z=321.1 [M+H⁺]

To a solution of 2-(8,11-dioxadispiro[3.2.47.24]tridecan-2-yloxy) pyrimidine-5-carboxylic acid (35 mg), 4-(3-amino-2,2,4,4-tetramethyl-cyclobutoxy)-2-chloro-benzonitrile (38 mg), DIPEA (111 mg, 861 μmol, 150 μL, 7.9 eq) in DMF (0.5 mL) was added T₄P (160 mg, 222 μmol, 50% purity, 2.0 eq). The mixture was stirred at 25° C. for 12 h. The mixture was poured into water 50 mL, Ethyl acetate (50 mL×3) was added to the mixture and the organic phase was separated, the resulting mixture was washed with brine (50 mL×2). The combined organic phase was dried over sodium sulfate, filtered and concentrated in vacuum. Compound N-[3-(3-chloro-4-cyano-phenoxy)-2,2,4,4-tetramethyl-cyclobutyl]-2-(8,11-dioxadispiro[3.2.47.24]tridecan-2-yloxy) pyrimidine-5-carboxamide (45 mg) was obtained as a yellow solid and directly used in the next step without further purification.

LC-MS: MS (ESI⁺): t_{R}=0.700 min, m/z=581.3 [M+H⁺]

To a solution of N-[3-(3-chloro-4-cyano-phenoxy)-2,2,4,4-tetramethyl-cyclobutyl]-2-(8,11-dioxadispiro[3.2.47.24]tridecan-2-yloxy) pyrimidine-5-carboxamide (45 mg) in DCM (1 mL) was added TFA (110 mg, 966 μmol, 71.4 μL, 12 eq). The mixture was stirred at 25° C. for 1 h. The mixture was poured into water 50 mL, Ethyl acetate (50 mL×3) was added to the mixture and the organic phase was separated, the resulting mixture was washed with brine (50 mL×2). The combined organic phase was dried over sodium sulfate, filtered and concentrated in vacuum. The crude product was purified by reversed-phase HPLC (column: Phenomenex luna C18 150×25 mm×10 um; mobile phase: [water (FA)-ACN]; gradient: 50%-80% B over 10 min). Compound N-[3-(3-chloro-4-cyano-phenoxy)-2,2,4,4-tetramethyl-cyclobutyl]-2-(7-oxospiro[3,5]nonan-2-yl) oxy-pyrimidine-5-carboxamide (32 mg, 59.6 μmol, 55% yield over three steps) was obtained as a white solid.

LC-MS: MS (ESI⁺): t_{R}=0.655 min, m/z=537.3 [M+H⁺]

To a solution of N-[3-(3-chloro-4-cyano-phenoxy)-2,2,4,4-tetramethyl-cyclobutyl]-2-(7-oxospiro[3.5]nonan-2-yl)oxy-pyrimidine-5-carboxamide (32 mg, 59.6 μmol, 1.0 eq), 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (31 mg, 60.8 μmol, 1.0 eq) in DCE (0.3 mL) and DMSO (0.3 mL) was added Ti(Oi-Pr)₄ (254 mg, 894 μmol, 263 μL, 15 eq) at 25° C. The mixture was stirred at 25° C. for 2 h. The mixture was added NaBH(OAc)₃ (189 mg, 894 μmol, 15 eq) and stirred at 25° C. for 2 h. The reaction mixture was concentrated under reduced pressure to give a residue. The crude product was purified by reversed-phase HPLC (column: Phenomenex luna C18 150×25 mm×10 um; mobile phase: [water (FA)-ACN]; gradient: 32%-62% B over 10 mi). Compound 2-[7-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]spiro[3.5]nonan-2-yl]oxy-N-[3-(3-chloro-4-cyano-phenoxy)-2,2,4,4-tetramethyl-cyclobutyl]pyrimidine-5-carboxamide (16.63 mg, 15.97 μmol, 27% yield) was obtained as a white solid.

¹H NMR (400 MHz, DMSO-d₆) δ=8.95 (s, 2H), 8.07 (d, J=9.6 Hz, 1H), 7.90 (d, J=8.8 Hz, 1H), 7.75 (s, 1H), 7.50 (s, 1H), 7.22 (d, J=2.6 Hz, 1H), 7.12 (s, 1H), 7.04-6.99 (m, 1H), 6.97-6.62 (m, 2H), 5.27-5.12 (m, 1H), 4.49-4.24 (m, 2H), 4.21-4.09 (m, 2H), 4.05 (d, J=9.2 Hz, 1H), 3.86 (s, 3H), 3.78-3.67 (m, 2H), 3.62-3.56 (m, 2H), 3.29-3.22 (m, 2H), 2.88-2.73 (m, 5H), 2.34-2.27 (m, 2H), 2.19-1.70 (m, 15H), 1.58-1.36 (m, 6H), 1.23 (s, 6H), 1.12 (s, 6H).

LC-MS: MS (ESI⁺): t_{R}=3.143 min m/z=1030.7 [M+H⁺]

The compounds below were prepared in a similar manner as described in Example 59.

| | | | LC-MS |
|---|---|---|---|
| Compound | Name | 1H NMR (DMSO-d6) | [M + H⁺] |
| 384 | 4-[7-[4-[5-acetyl-3-[7- | δ = 8.38 (s, 1H), 7.80 (d, J = 8.8 Hz, 2H), | 1024.8 |
| | (difluoromethyl)-6-(1- | 7.75 (s, 1H), 7.69-7.62 (m, 2H), 7.49 (s, | |
| | methylpyrazol-4-yl)-3,4- | 1H), 7.10 (s, 1H), 6.90 (d, J = 8.8 Hz, | |
| | dihydro-2H-quinolin-1-yl]-6,7- | 2H), 6.79-6.51 (m, 4H), 4.77 (br t, J = | |
| | dihydro-4H-pyrazolo[4,3-c] | 6.8 Hz, 1H), 4.26 (s, 1H), 4.16-4.08 (m, | |
| | pyridin-1-yl]-1-piperidyl] spiro | 2H), 4.04 (d, J = 9.0 Hz, 1H), 3.96 (br d, | |
| | [3.5] nonan-2-yl] oxy-N-[3-(4- | J = 4.6 Hz, 1H), 3.88 (d, J = 16.6 Hz, | |
| | cyano-3-methoxy-phenoxy)- | 6H), 3.74-3.66 (m, 2H), 3.63-3.55 (m, | |
| | 2,2,4,4-tetramethyl-cyclobutyl] | 3H), 2.96-2.80 (m, 6H), 2.77-2.69 (m, | |
| | benzamide | 1H), 2.28 (br d, J = 9.6 Hz, 3H), 2.07 (s, | |
| | | 2H), 1.99-1.91 (m, 5H), 1.88-1.76 (m, | |
| | | 4H), 1.74-1.58 (m, 4H), 1.45-1.29 (m, | |
| | | 4H), 1.22 (s, 6H), 1.14 (s, 6H) | |
| 385 | 2-[6-[4-[5-acetyl-3-[7- | δ = 9.00-8.90 (m, 2H), 8.11-8.02 (m, | 1002.7 |
| | (difluoromethyl)-6-(1- | 1H), 7.91 (d, J = 8.8 Hz, 1H), 7.76 (s, | |
| | methylpyrazol-4-yl)-3,4- | 1H), 7.51 (s, 1H), 7.28-7.19 (m, 1H), | |
| | dihydro-2H-quinolin-1-yl]-6,7- | 7.14-7.10 (m, 1H), 7.05-7.00 (m, 1H), | |
| | dihydro-4H-pyrazolo[4,3- | 6.96-6.65 (m, 2H), 5.19-5.07 (m, 1H), | |
| | c]pyridin-1-yl]-1- | 4.35-4.28 (m, 1H), 4.20-3.99 (m, 4H), | |
| | piperidyl]spiro[3.3]heptan-2- | 3.76-3.66 (m, 2H), 3.64-3.56 (m, 2H), | |
| | yl]oxy-N-[3-(3-chloro-4-cyano- | 3.40 (br s, 2H), 2.93-2.80 (m, 6H), 2.76- | |
| | phenoxy)-2,2,4,4-tetramethyl- | 2.69 (m, 1H), 2.65-2.59 (m, 1H), 2.45- | |
| | cyclobutyl]pyrimidine-5- | 2.41 (m, 1H), 2.26-2.15 (m, 2H), 2.13- | |
| | carboxamide | 2.05 (m, 4H), 2.04-1.94 (m, 5H), 1.94- | |
| | | 1.80 (m, 7H), 1.23 (s, 6H), 1.16-1.09 | |
| | | (m, 6H) | |
| 386 | 4-[6-[4-[5-acetyl-3-[7- | δ = 7.81-7.79 (d, J = 8.8 Hz, 2H), 7.75 | 996.8 |
| | (difluoromethyl)-6-(1- | (s, 1H), 7.68-7.66 (d, J = 8.8 Hz, 2H), | |
| | methylpyrazol-4-yl)-3,4- | 7.50 (s, 1H), 7.11 (s, 1H), 6.91-6.80 (m, | |
| | dihydro-2H-quinolin-1-yl]-6,7- | 4H), 6.65-6.64 (d, J = 2.0 Hz, 1H), 6.56- | |
| | dihydro-4H-pyrazolo[4,3- | 6.53 (m, 1H), 4.73-4.68 (m, 1H), 4.27 | |
| | c]pyridin-1-yl]-1- | (s, 1H), 4.17-4.12 (m, 2H), 4.06-4.04 | |
| | piperidyl]spiro[3.3]heptan-2- | (m, 2H), 3.91-3.87 (m, 6H), 3.69 (m, | |
| | yl]oxy-N-[3-(4-cyano-3- | 2H), 3.60-3.57 (m, 2H), 2.85 (m, 6H), | |
| | methoxy-phenoxy)-2,2,4,4- | 2.67-2.65 (m, 3H), 2.10-2.08 (m, 4H), | |
| | tetramethyl- | 2.00-1.96 (m, 6H), 1.88-1.83 (m, 6H), | |
| | cyclobutyl]benzamide | 1.23 (s, 6H), 1.15 (s, 6H) | |
| 395 | 2-[2-[4-[5-acetyl-3-[7- | (CDCl₃) δ = 8.84 (s, 2H), 7.52-7.42 (m, | 1030.7 |
| | (difluoromethyl)-6-(1- | 2H), 7.33 (d, J = 5.6 Hz, 1H), 7.03-6.83 | |
| | methylpyrazol-4-yl)-3,4- | (m, 3H), 6.74 (dd, J = 2.4, 8.8 Hz, 1H), | |
| | dihydro-2H-quinolin-1-yl]-6,7- | 6.64-6.31 (m, 1H), 6.20 (br d, J = 8.0 | |
| | dihydro-4H-pyrazolo[4,3- | Hz, 1H), 5.00 (br d, J = 3.2 Hz, 1H), 4.39- | |
| | c]pyridin-1-yl]-1- | 4.19 (m, 2H), 4.15-4.05 (m, 2H), 4.02 | |
| | piperidyl]spiro[3.5]nonan-7- | (s, 1H), 3.94-3.78 (m, 4H), 3.75-3.47 | |
| | yl]oxy-N-[3-(3-chloro-4-cyano- | (m, 4H), 3.43-3.00 (m, 4H), 2.87-2.58 | |
| | phenoxy)-2,2,4,4-tetramethyl- | (m, 6H), 2.50-2.22 (m, 3H), 2.10 (s, | |
| | cyclobutyl]pyrimidine-5- | 2H), 2.05-1.97 (m, 5H), 1.85-1.41 (m, | |
| | carboxamide | 9H), 1.21 (s, 6H), 1.18-1.13 (m, 6H) | |
| 396 | 4-[2-[4-[5-acetyl-3-[7- | (CDCl₃) δ = 8.50 (s, 1H), 7.65 (d, J = 8.8 | 1024.8 |
| | (difluoromethyl)-6-(1- | Hz, 2H), 7.50-7.43 (m, 1H), 7.38 (d, J = | |
| | methylpyrazol-4-yl)-3,4- | 8.8 Hz, 1H), 7.33 (d, J = 7.2 Hz, 1H), | |
| | dihydro-2H-quinolin-1-yl]-6,7- | 7.00-6.76 (m, 4H), 6.64-6.41 (m, 1H), | |
| | dihydro-4H-pyrazolo[4,3- | 6.39 (d, J = 2.0 Hz, 1H), 6.35-6.29 (m, | |
| | c]pyridin-1-yl]-1- | 1H), 6.06 (d, J = 8.0 Hz, 1H), 4.25 (br d, | |
| | piperidyl]spiro[3.5]nonan-7- | J = 3.2 Hz, 1H), 4.17 (s, 1H), 4.10-4.02 | |
| | yl]oxy-N-[3-(4-cyano-3- | (m, 2H), 3.98 (s, 1H), 3.93-3.79 (m, | |
| | methoxy-phenoxy)-2,2,4,4- | 8H), 3.73-3.54 (m, 3H), 2.97 (br d, J = | |
| | tetramethyl- | 7.2 Hz, 2H), 2.84-2.60 (m, 5H), 2.17 (br | |
| | cyclobutyl]benzamide | d, J = 9.6 Hz, 2H), 2.08 (s, 1H), 2.04- | |
| | | 1.95 (m, 6H), 1.81-1.47 (m, 10H), 1.46- | |
| | | 1.37 (m, 2H), 1.18 (d, J = 10.4 Hz, | |
| | | 12H) | |

### Example 60: Synthesis of 2-[(3S)-3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]-1-piperidyl]-N-[3-(3-chloro-4-cyano-phenoxy)-2,2,4,4-tetramethyl-cyclobutyl]pyrimidine-5-carboxamide (Compound 397)

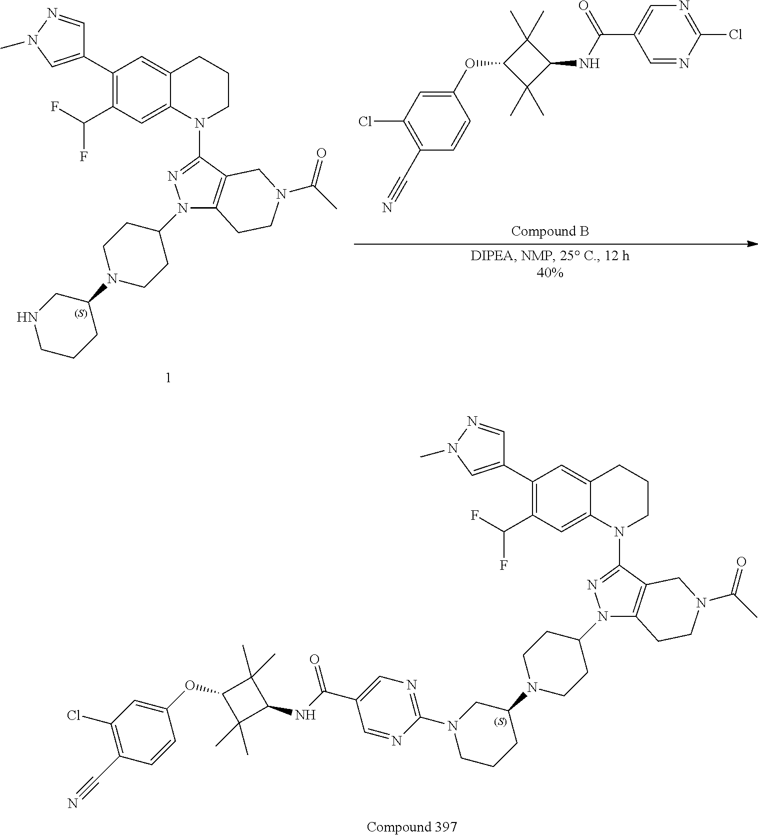

To a mixture of 1-[3-[7-(difluoromethyl)-6-(1-methylpyrazol-4-yl)-3,4-dihydro-2H-quinolin-1-yl]-1-[1-[(3S)-3-piperidyl]-4-piperidyl]-6,7-dihydro-4H-pyrazolo[4,3-c]pyridin-5-yl]ethanone (60 mg, 93 mol, 1.0 eq) and 2-chloro-N-[3-(3-chloro-4-cyano-phenoxy)-2,2,4,4-tetramethyl-cyclobutyl]pyrimidine-5-carboxamide (39 mg, 93 μmol, 1.0 eq) in NMP (1 my) was added DIPEA (60 mg, 469 μmol, 81 μL, 5.0 eq), the mixture was stirred at 25° C. for 12 h. The mixture was filtered and concentrated to give a residue. The residue was purified by prep-HPLC (column: Unisil 3-100 C18 Ultra 150*50 mm*3 um; mobile phase: [water(FA)-ACN]; gradient: 30%-50% B over 10 min). Compound 2-[(3S)-3-[4-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]-1-piperidyl]-1-piperidyl]-N-[3-(3-chloro-4-cyano-phenoxy)-2, 2,4,4-tetramethyl-cyclobutyl]pyrimidine-5-carboxamide (38.2 mg, 38.48 mol, 40% yield) was obtained as a yellow solid.

¹H NMR (400 MHz, DMSO-d₆) δ=8.78 (s, 2H), 7.94-7.86 (m, 1H), 7.84-7.68 (m, 2H), 7.50 (s, 1H), 7.25-7.18 (m, 1H), 7.15-7.08 (m, 1H), 7.05-6.98 (m, 1H), 6.97-6.61 (m, 2H), 5.02-4.69 (m, 1H), 4.66-4.47 (m, 1H), 4.29 (s, 1H), 4.21-4.09 (m, 2H), 4.08-4.00 (m, 1H), 3.87 (s, 3H), 3.80-3.67 (m, 2H), 3.65-3.56 (m, 2H), 3.30-2.96 (m, 5H), 2.93-2.70 (m, 5H), 2.61-2.54 (m, 1H), 2.21-1.62 (m, 12H), 1.53-1.34 (m, 1H), 1.27-1.18 (m, 6H), 1.16-1.07 (m, 6H).

LC-MS: MS (ESI⁺): t_{R}=3.047 min, m/z=975.6 [M+H⁺]

SFC: t₁=1.375 min, 100%

The compounds below were prepared in a similar manner as described in Example 60.

| | | | LC-MS |
|---|---|---|---|
| Compound | Name | ¹H NMR (CDCl₃) | [M + H⁺] |
| 398 | 2-[(3R)-3-[4-[5-acetyl-3-[7- | δ = 8.70 (s, 2H), 7.61-7.50 (m, 2H), | 975.6 |
| | (difluoromethyl)-6-(1- | 7.44-7.37 (m, 1H), 7.09-6.94 (m, 2H), | |
| | methylpyrazol-4-yl)-3,4- | 6.90-6.77 (m, 2H), 6.71-6.36 (m, 1H), | |
| | dihydro-2H-quinolin-1-yl]-6,7- | 5.94 (d, J = 8.0 Hz, 1H), 4.99-4.84 (m, | |
| | dihydro-4H-pyrazolo[4,3- | 1H), 4.75-4.62 (m, 1H), 4.29-4.08 (m, | |
| | c]pyridin-1-yl]-1-piperidyl]-1- | 3H), 4.04 (s, 1H), 4.00-3.86 (m, 5H), | |
| | piperidyl]-N-[3-(3-chloro-4- | 3.79-3.66 (m, 3H), 3.32-3.10 (m, 2H), | |
| | cyano-phenoxy)-2,2,4,4- | 3.06-2.93 (m, 2H), 2.92-2.79 (m, 3H), | |
| | tetramethyl- | 2.78-2.71 (m, 1H), 2.54-2.33 (m, 3H), | |
| | cyclobutyl]pyrimidine-5- | 2.31-2.19 (m, 2H), 2.17 (s, 1H), 2.12- | |
| | carboxamide | 2.01 (m, 5H), 2.00-1.85 (m, 3H), 1.60- | |
| | | 1.45 (m, 2H), 1.30-1.19 (m, 12H) | |
| 399 | 4-[(3S)-3-[4-[5-acetyl-3-[7- | (DMSO-d₆) δ = 7.83-7.69 (m, 3H), 7.68- | 969.7 |
| | (difluoromethyl)-6-(1- | 7.60 (m, 1H), 7.54-7.45 (m, 2H), 7.16- | |
| | methylpyrazol-4-yl)-3,4- | 7.08 (m, 1H), 6.99-6.61 (m, 5H), 6.58- | |
| | dihydro-2H-quinolin-1-yl]-6,7- | 6.49 (m, 1H), 4.27 (s, 1H), 4.21-4.09 | |
| | dihydro-4H-pyrazolo[4,3- | (m, 2H), 4.07-3.97 (m, 2H), 3.95-3.89 | |
| | c]pyridin-1-yl]-1-piperidyl]-1- | (m, 4H), 3.86 (s, 3H), 3.84-3.78 (m, | |
| | piperidyl]-N-[3-(4-cyano-3- | 1H), 3.77-3.67 (m, 2H), 3.63-3.56 (m, | |
| | methoxy-phenoxy)-2,2,4,4- | 2H), 3.47-3.41 (m, 1H), 3.10-2.96 (m, | |
| | tetramethyl- | 2H), 2.90-2.69 (m, 6H), 2.47-2.37 (m, | |
| | cyclobutyl]benzamide | 2H), 2.11-2.04 (m, 2H), 2.04-1.84 (m, | |
| | | 8H), 1.81-1.72 (m, 1H), 1.58-1.41 (m, | |
| | | 2H), 1.26-1.19 (m, 6H), 1.18-1.11 (m, | |
| | | 6H) | |
| 400 | 4-[(3R)-3-[4-[5-acetyl-3-[7- | δ = 7.69 (d, J = 8.8 Hz, 2H), 7.53 (d, J = | 969.9 |
| | (difluoromethyl)-6-(1- | 6.4 Hz, 1H), 7.46 (d, J = 8.8 Hz, 1H), | |
| | methylpyrazol-4-yl)-3,4- | 7.43-7.37 (m, 1H), 7.07-6.99 (m, 1H), | |
| | dihydro-2H-quinolin-1-yl]-6,7- | 6.91 (d, J = 9.2 Hz, 2H), 6.88-6.83 (m, | |
| | dihydro-4H-pyrazolo[4,3- | 1H), 6.76-6.50 (m, 1H), 6.47 (d, J = 2.0 | |
| | c]pyridin-1-yl]-1-piperidyl]-1- | Hz, 1H), 6.43-6.37 (m, 1H), 6.12 (d, J = | |
| | piperidyl]-N-[3-(4-cyano-3- | 8.0 Hz, 1H), 4.26 (s, 1H), 4.20-4.09 (m, | |
| | methoxy-phenoxy)-2,2,4,4- | 2H), 4.05 (s, 1H), 3.96 (d, J = 2.4 Hz, | |
| | tetramethyl- | 5H), 3.94-3.89 (m, 4H), 3.82-3.68 (m, | |
| | cyclobutyl]benzamide | 4H), 3.28-3.10 (m, 2H), 2.94-2.78 (m, | |
| | | 4H), 2.78-2.67 (m, 3H), 2.63-2.46 (m, | |
| | | 2H), 2.30-2.20 (m, 2H), 2.17 (s, 1H), | |
| | | 2.11-2.00 (m, 6H), 1.91-1.84 (m, 2H), | |
| | | 1.51 (br d, J = 11.6 Hz, 2H), 1.25 (d, J = | |
| | | 10.4 Hz, 12H) | |

### Example 61: Synthesis of 4-[3-[8-[5-acetyl-3-[7-(difluoromethyl)-6-(1-methylpyrazol-4-yl)-3,4-dihydro-2H-quinolin-1-yl]-6, 7-dihydro-4H-pyrazolo[4,3-c]pyridin-1-yl]-2-azaspiro [4.5]decan-2-yl]azetidin-1-yl]-N-[3-(4-cyano-3-methoxy-phenoxy)-2,2,4,4-tetramethyl-cyclobutyl]benzamide (Compound 401)

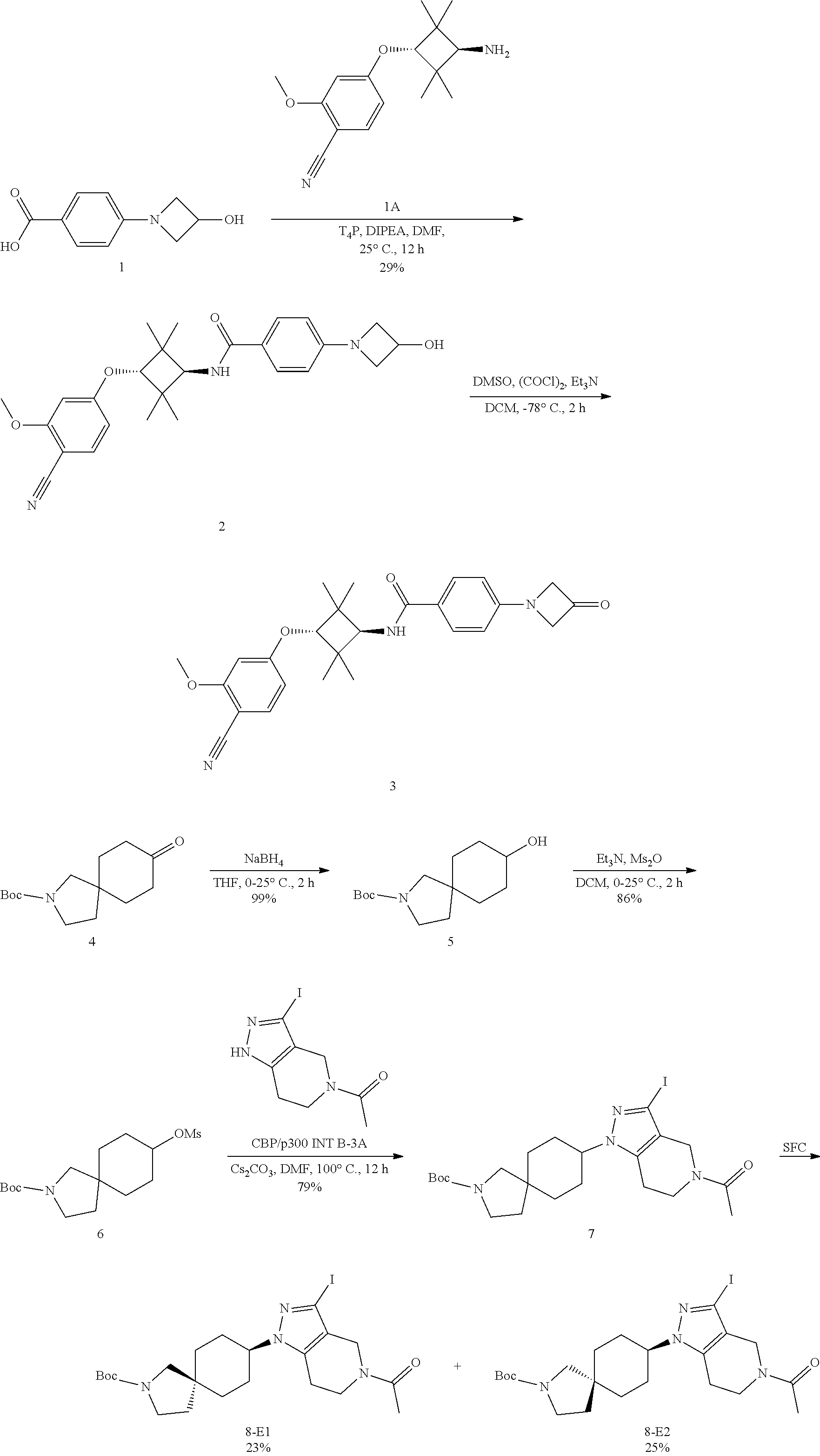

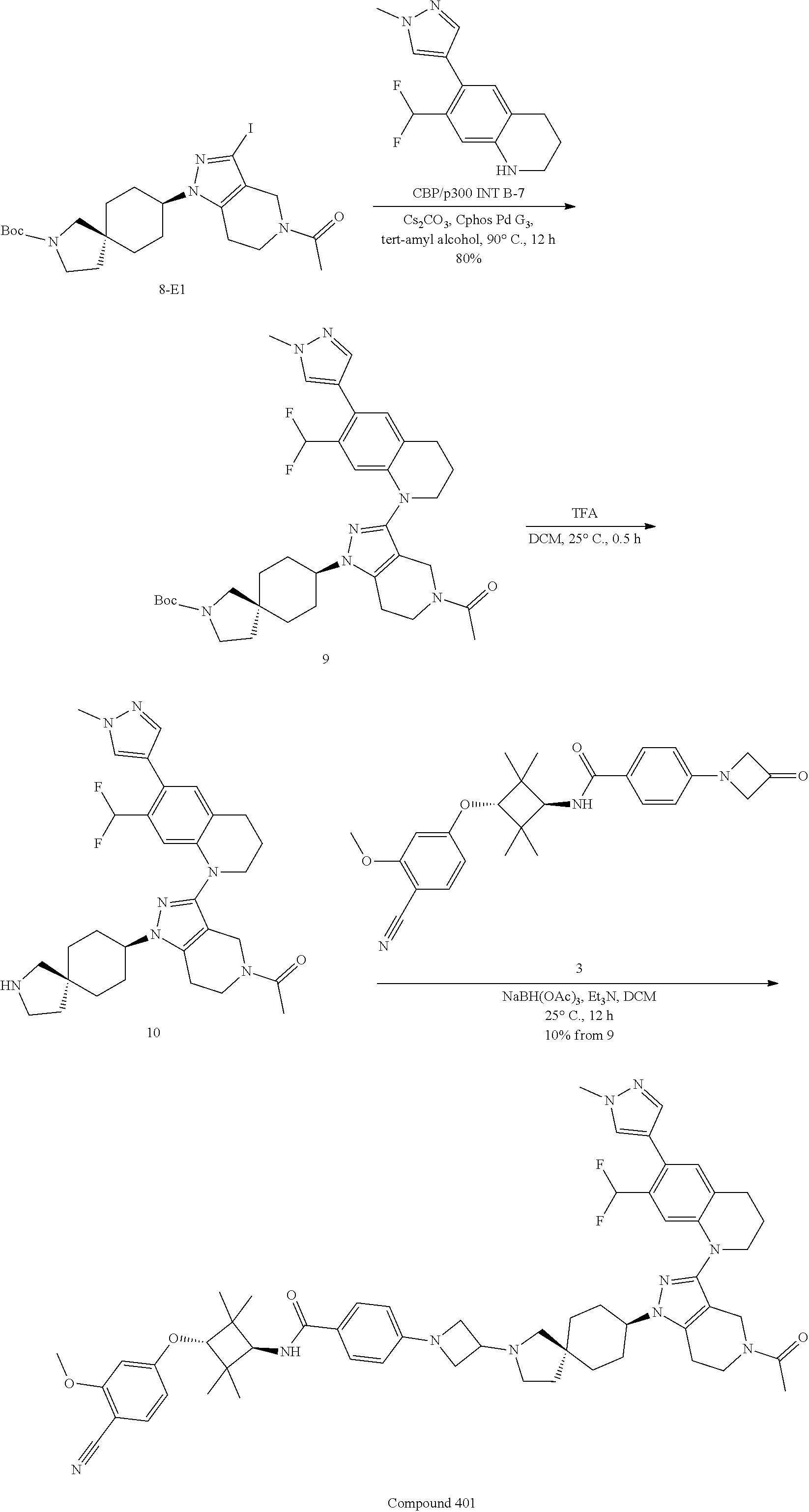

Synthesis of 1 was reported in WO2012/177606 A1.

Synthesis of 1A was reported in WO2021/127443 A1.

Synthesis of 4 was reported in WO2015/94803 A1.

A solution of 4-(3-hydroxyazetidin-1-yl)benzoic acid (700 mg, 2.28 mmol, 1.0 eq, TFA) and 4-(3-amino-2,2,4,4-tetramethyl-cyclobutoxy)-2-methoxy-benzonitrile (885 mg, 2.28 mmol, 1.0 eq, TFA) in DCM (10 mL) was added DIPEA (1.47 g, 11.39 mmol, 1.98 mL, 5.0 eq) and T₄P (3.28 g, 4.56 mmol, 50% purity, 2.0 eq). The mixture was stirred at 25° C. for 1 h. The reaction mixture was quenched by saturated ammonium chloride solution (30 mL) and extracted with EtOAc (50 mL×3). The combined organic layers were washed with brine (30 mL×2), dried over Na₂SO₄, filtered and concentrated under reduced pressure to give a residue. The residue was triturated with Petroleum ether/Ethyl acetate=10/1 at 25° C. for 1 h. Compound N-[3-(4-cyano-3-methoxy-phenoxy)-2,2,4,4-tetramethyl-cyclobutyl]-4-(3-hydroxyazetidin-1-yl)benzamide (300 mg, 667 μmol, 29.29% yield) was obtained as a white solid.

LC-MS: MS (ESI⁺: t_{R}=0.549 min, m/z=450.3 [M+H⁺]

A solution of (COCl)₂ (106 mg, 834 μmol, 73 μL, 1.5 eq) in DCM (3 mL) was cooled to −78° C., DMSO (130 mg, 1.67 mmol, 1305 μL, 3.0 eq) was added into the mixture at −78° C. and stirred at −78° C. for 0.5 h. N-[3-(4-cyano-3-methoxy-phenoxy)-2,2,4,4-tetramethyl-cyclobutyl]-4-(3-hydroxyazetidin-1-yl)benzamide (250 mg, 556 μmol, 1.0 eq) in DCM (3 mL) was added into the mixture at −78° C. and stirred at −78° C. for 1 h. Et₃N (338 mg, 3.34 mmol, 464 μL, 6.0 eq) was added into the mixture at −78° C. and stirred at −78° C. for 0.5 h. The mixture was quenched with water (10 mL), extracted with EtOAc (20 mL×3), washed with brine (20 mL), dried over Na₂SO₄, dried over Na₂SO₄, filtered and concentrated under reduced pressure to give a residue. The crude product was N-[3-(4-cyano-3-methoxy-phenoxy)-2,2,4,4-tetramethyl-cyclobutyl]-4-(3-oxoazetidin-1-yl)benzamide (250 mg) was obtained as a yellow solid and directly used in the next step without further purification.

LC-MS: MS (ESI⁺: t_{R}=0.584 min, m/z=448.1 [M+H⁺]

A solution of tert-butyl 8-oxo-2-azaspiro [4.5]decane-2-carboxylate (10.00 g, 39.47 mmol, 1.0 eq) in THF (100 mL) was added NaBH₄ (2.39 g, 63.17 mmol, 1.6 eq) at 0° C. under N₂ atmosphere. The mixture was stirred at 25° C. for 1 h. The reaction mixture was quenched by addition saturated ammonium chloride solution 100 mL, and then extracted with EtOAc (200 mL×2). The combined organic layers were washed with brine (500 mL), dried over Na₂SO₄, filtered and concentrated under reduced pressure to give a residue. The residue was purified by column chromatography (SiO₂, Petroleum ether/Ethyl acetate=10/1 to 1/1). Compound tert-butyl 8-hydroxy-2-azaspiro [4.5]decane-2-carboxylate (10.00 g, 39.16 mmol, 99% yield) was obtained as a colorless oil.

A solution of tert-butyl 8-hydroxy-2-azaspiro [4.5]decane-2-carboxylate (10.00 g, 39.16 mmol, 1.0 eq) in DCM (100 mL) was added Et₃N (11.89 g, 117.48 mmol, 16.4 mL, 3.0 eq) and methylsulfonyl methanesulfonate (13.64 g, 78.32 mmol, 2.0 eq) at 0° C. The mixture was stirred at 25° C. for 2 h. The reaction mixture was quenched by addition water 30 mL, and then extracted with DCM (20 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=20/1 to 10/1). Compound tert-butyl 8-methylsulfonyloxy-2-azaspiro [4.5]decane-2-carboxylate (11.20 g, 33.59 mmol, 86% yield) was obtained as a colorless oil.

A solution of tert-butyl 8-methylsulfonyloxy-2-azaspiro[4.5]decane-2-carboxylate (11.20 g, 33.59 mmol, 1.0 eq) in DMF (200 mL) was added Cs₂CO₃ (32.83 g, 100.77 mmol, 3.0 eq) and 1-(3-iodo-1,4,6,7-tetrahydropyrazolo[4,3-c]pyridin-5-yl)ethanone (9.78 g, 33.59 mmol, 1.0 eq). The mixture was stirred at 100° C. for 12 h. The reaction mixture was quenched by addition water 100 mL, and then extracted with EtOAc (100 mL×2). The combined organic layers were washed with brine (200 mL×2), dried over Na₂SO₄, filtered and concentrated under reduced pressure to give a residue. The residue was purified by column chromatography (SiO₂, Petroleum ether/Ethyl acetate=1/1 to 1/3). Compound tert-butyl 8-(5-acetyl-3-iodo-6,7-dihydro-4H-pyrazolo[4,3-c]pyridin-1-yl)-2-azaspiro[4.5]decane-2-carboxylate (14.00 g, 26.49 mmol, 79% yield) was obtained as a white solid.

LC-MS: MS (ESI⁺): t_{R}=0.581 min, m/z=529.1 [M+H⁺]

Tert-butyl 8-(5-acetyl-3-iodo-6,7-dihydro-4H-pyrazolo[4,3-c]pyridin-1-yl)-2-azaspiro[4.5]decane-2-carboxylate (14.00 g, 26.49 mmol, 1.0 eq) was separated by SFC three times: the first method (column: DAICEL CHIRALPAK AD (250 mm*50 mm, 10 um); mobile phase: [CO₂-EtOH (0.1% NH₃H₂O)]; B %: 32%, isocratic elution mode), the second method (column: DAICEL CHIRALCEL OJ (250 mm*30 mm, 10 um); mobile phase: [CO₂-EtOH (0.1% NH₃H₂O)]; B %: 20%, isocratic elution mode), the third method (column: DAICEL CHIRALCEL OD (250 mm*30 mm, 10 um); mobile phase: [CO₂-EtOH (0.1% NH₃H₂O)]; B %: 30%, isocratic elution mode). Compound tert-butyl 8-(5-acetyl-3-iodo-6,7-dihydro-4H-pyrazolo[4,3-c]pyridin-1-yl)-2-azaspiro[4.5]decane-2-carboxylate (3.25 g, 6.01 mmol, 23% yield) was obtained as a white solid. Compound tert-butyl 8-(5-acetyl-3-iodo-6,7-dihydro-4H-pyrazolo[4,3-c]pyridin-1-yl)-2-azaspiro[4.5]decane-2-carboxylate (3.55 g, 6.65 mmol, 25% yield) was obtained as a white solid.

¹H NMR (400 MHz, DMSO-d₆) δ=4.28-4.21 (m, 2H), 4.16-4.05 (m, 1H), 3.80-3.69 (m, 2H), 3.36-3.28 (m, 2H), 3.25-3.19 (m, 2H), 2.86-2.67 (m, 2H), 2.16-2.09 (m, 3H), 1.87-1.75 (m, 4H), 1.73-1.64 (m, 4H), 1.55-1.42 (m, 11H)

LC-MS: MS (ESI⁺): t_{R}=0.573 min, m/z=473.0 [M-tBu⁺]

SFC: t_{R}=1.281 min, 98.004%

¹H NMR (400 MHz, DMSO-d₆) δ=4.28-4.22 (m, 2H), 4.14-4.07 (m, 1H), 3.80-3.69 (m, 2H), 3.38-3.31 (m, 2H), 3.06 (s, 2H), 2.87-2.68 (m, 2H), 2.16-2.10 (m, 3H), 1.96-1.74 (m, 6H), 1.71-1.62 (m, 2H), 1.59-1.48 (m, 2H), 1.44 (s, 9H)

LC-MS: MS (ESI⁺): t_{R}=0.578 min, m/z=529.0 [M+H⁺]

SFC: t_{R}=1.839 min, 99.338%

A mixture of tert-butyl 8-(5-acetyl-3-iodo-6,7-dihydro-4H-pyrazolo[4,3-c]pyridin-1-yl)-2-azaspiro[4.5]decane-2-carboxylate (1.00 g, 1.89 mmol, 1.0 eq), 7-(difluoromethyl)-6-(1-methylpyrazol-4-yl)-1,2,3,4-tetrahydroquinoline (598 mg, 2.27 mmol, 1.2 eq), Cphos Pd G3 (153 mg, 189 μmol, 0.1 eq), Cs₂CO₃ (2.47 g, 7.57 mmol, 4.0 eq) in 2-methylbutan-2-ol (15 mL) was degassed and purged with N₂ for 3 times, and then the mixture was stirred at 90° C. for 12 h under N₂ atmosphere. The reaction mixture was quenched by addition water 30 mL, and then extracted with EtOAc (20 mL×2). The combined organic layers were washed with brine (50 mL×2), dried over Na₂SO₄, filtered and concentrated under reduced pressure to give a residue. The residue was purified by column chromatography (SiO₂, Petroleum ether/THF=10/1 to 1/2). Compound tert-butyl 8-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[4.5]decane-2-carboxylate (1.00 g, 1.51 mmol, 80% yield) was obtained as a yellow solid.

To a solution of tert-butyl 8-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[4.5]decane-2-carboxylate (100 mg, 151 μmol, 1.0 eq) in DCM (1 mL) was added TFA (1 mL). The mixture was stirred at 25° C. for 0.5 h. The reaction mixture was concentrated under reduced pressure to remove solvent. Compound 1-[1-(2-azaspiro[4.5]decan-8-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (80 mg) was obtained as a yellow oil and directly used in the next step without further purification.

A solution of 1-[1-(2-azaspiro[4.5]decan-8-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (80 mg) in DCM (2 mL) was added Et₃N (43 mg, 425 μmol, 59 μL, 3.0 eq), N-[3-(4-cyano-3-methoxy-phenoxy)-2,2,4,4-tetramethyl-cyclobutyl]-4-(3-oxoazetidin-1-yl)benzamide (95 mg, 213 μmol, 1.5 eq) and NaBH(OAc)₃ (150 mg, 709 μmol, 5.0 eq). The mixture was stirred at 25° C. for 12 h. The reaction mixture was quenched by addition water 30 mL, and then extracted with EtOAc (20 mL×2). The combined organic layers were washed with brine (50 mL×2), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 33%-63% B over 11 min). Compound 4-[3-[8-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[4.5]decan-2-yl]azetidin-1-yl]-N-[3-(4-cyano-3-methoxy-phenoxy)-2,2,4,4-tetramethyl-cyclobutyl]benzamide (14.23 mg, 14.30 mol, 10.07% yield over two steps) was obtained as a white solid,

¹H NMR (400 MHz, DMSO-d₆) δ=8.43-8.38 (m, 1H), 7.78-7.69 (m, 3H), 7.65 (d, J=8.6 Hz, 1H), 7.51 (s, 1H), 7.45 (br d, J=9.4 Hz, 1H), 7.14-7.07 (m, 1H), 6.96-6.61 (m, 3H), 6.58-6.51 (m, 1H), 6.48-6.37 (m, 2H), 4.30-4.24 (m, 1H), 4.22-4.11 (m, 2H), 4.08-4.01 (m, 2H), 3.97 (br t, J=7.4 Hz, 2H), 3.89 (d, J=12.8 Hz, 6H), 3.77-3.67 (m, 4H), 3.61-3.54 (m, 2H), 3.48-3.43 (m, 2H), 2.88-2.71 (m, 4H), 2.58-2.55 (m, 1H), 2.48-2.43 (m, 2H), 2.11-2.06 (m, 2H), 1.97 (s, 3H), 1.85-1.70 (m, 6H), 1.62-1.55 (m, 2H), 1.54-1.44 (m, 2H), 1.22 (s, 6H), 1.14 (s, 6H)

LC-MS: MS (ESI⁺) t_{R}=3.072 min, m/z=995.7 [M+H⁺]

The compounds below were prepared in a similar manner as described in Example 61.

| | | | LC-MS |
|---|---|---|---|
| Compound | Name | ¹H NMR (DMSO-d6) | [M + H⁺] |
| 402 | 4-[3-[8-[5-acetyl-3-[7- | δ = 8.51-8.45 (m, 1H), 7.79-7.71 (m, | 995.6 |
| | (difluoromethyl)-6-(1- | 3H), 7.65 (d, J = 8.4 Hz, 1H), 7.55-7.41 | |
| | methylpyrazol-4-yl)-3,4- | (m, 2H), 7.14-7.05 (m, 1H), 6.95-6.62 | |
| | dihydro-2H-quinolin-1-yl]-6,7- | (m, 3H), 6.58-6.50 (m, 1H), 6.47-6.40 | |
| | dihydro-4Hpyrazolo[4,3- | (m, 2H), 4.27 (s, 1H), 4.22-4.11 (m, | |
| | c]pyridin-1-yl]-2- | 2H), 4.09-4.04 (m, 1H), 4.03-3.94 (m, | |
| | azaspiro[4.5]decan-2- | 3H), 3.89 (d, J = 17.2 Hz, 6H), 3.77- | |
| | yl]azetidin-1-yl]-N-[3-(4- | 3.66 (m, 4H), 3.63-3.55 (m, 2H), 3.45- | |
| | cyano-3-methoxy-phenoxy)- | 3.37 (m, 2H), 2.90-2.70 (m, 4H), 2.60- | |
| | 2,2,4,4-tetramethyl- | 2.56 (m, 2H), 2.38-2.29 (m, 2H), 2.08 | |
| | cyclobutyl]benzamide | (s, 2H), 1.97 (s, 3H), 1.90-1.78 (m, 3H), | |
| | | 1.73-1.61 (m, 4H), 1.57-1.45 (m, 2H), | |
| | | 1.23 (s, 6H), 1.15 (s, 6H). | |
| 405 | 4-[8-[5-acetyl-3-[7- | δ = 7.78-7.71 (m, 3H), 7.65 (d, J = 8.4 | 940.9 |
| | (difluoromethyl)-6-(1- | Hz, 1H), 7.53-7.49 (m, 1H), 7.38 (d, J = | |
| | methylpyrazol-4-yl)-3,4- | 9.2 Hz, 1H), 7.11 (s, 1H), 6.95-6.65 (m, | |
| | dihydro-2H-quinolin-1-yl]-6,7- | 2H), 6.64 (d, J = 2.0 Hz, 1H), 6.62-6.52 | |
| | dihydro-4H-pyrazolo[4,3- | (m, 3H), 4.30-4.25 (m, 1H), 4.22-4.15 | |
| | c]pyridin-1-yl]-2- | (m, 2H), 4.14-4.02 (m, 2H), 3.91 (s, | |
| | azaspiro[4.5]decan-2-yl]-N-[3- | 3H), 3.87 (s, 3H), 3.79-3.68 (m, 2H), | |
| | (4-cyano-3-methoxy-phenoxy)- | 3.65-3.57 (m, 2H), 3.40-3.34 (m, 3H), | |
| | 2,2,4,4-tetramethyl- | 3.30-3.24 (m, 2H), 2.91-2.73 (m, 4H), | |
| | cyclobutyl]benzamide | 2.09 (s, 2H), 2.03-1.96 (m, 3H), 1.92- | |
| | | 1.83 (m, 5H), 1.77 (br d, J = 13.0 Hz, | |
| | | 2H), 1.64-1.53 (m, 2H), 1.27-1.19 (m, | |
| | | 6H), 1.18-1.11 (m, 6H) | |
| 406 | 4-[8-[5-acetyl-3-[7- | δ = 7.78-7.70 (m, 3H), 7.65 (d, J = 8.4 | 940.6 |
| | (difluoromethyl)-6-(1- | Hz, 1H), 7.50 (s, 1H), 7.40 (d, J = 9.2 | |
| | methylpyrazol-4-yl)-3,4- | Hz, 1H), 7.10 (s, 1H), 6.95-6.61 (m, | |
| | dihydro-2H-quinolin-1-yl]-6,7- | 3H), 6.57-6.48 (m, 3H), 4.27 (s, 1H), | |
| | dihydro-4H-pyrazolo[4,3- | 4.20-4.12 (m, 2H), 4.05 (d, J = 9.2 Hz, | |
| | c]pyridin-1-yl]-2- | 2H), 3.91 (s, 3H), 3.87 (s, 3H), 3.78- | |
| | azaspiro[4.5]decan-2-yl]-N-[3- | 3.66 (m, 2H), 3.63-3.56 (m, 2H), 3.40- | |
| | (4-cyano-3-methoxy-phenoxy)- | 3.36 (m, 2H), 3.13 (s, 2H), 2.90-2.71 | |
| | 2,2,4,4-tetramethyl- | (m, 4H), 2.08 (s, 2H), 1.97 (s, 7H), 1.88- | |
| | cyclobutyl]benzamide | 1.80 (m, 2H), 1.78-1.69 (m, 2H), 1.66- | |
| | | 1.55 (m, 2H), 1.22 (s, 6H), 1.14 (s, 6H) | |
| 407 | 2-[8-[5-acetyl-3-[7- | δ = 8.75 (br d, J = 8.0 Hz, 2H), 8.42- | 946.8 |
| | (difluoromethyl)-6-(1- | 8.38 (m, 1H), 7.90 (d, J = 8.8 Hz, 1H), | |
| | methylpyrazol-4-yl)-3,4- | 7.76 (s, 1H), 7.69 (d, J = 9.2 Hz, 1H), | |
| | dihydro-2H-quinolin-1-yl]-6,7- | 7.51 (s, 1H), 7.21 (d, J = 2.4 Hz, 1H), | |
| | dihydro-4H-pyrazolo[4,3- | 7.12 (s, 1H), 7.01 (dd, J = 2.4, 8.8 Hz, | |
| | c]pyridin-1-yl]-2- | 1H), 6.96-6.62 (m, 2H), 4.29 (s, 1H), | |
| | azaspiro[4.5]decan-2-yl]-N-[3- | 4.21-4.07 (m, 3H), 4.03 (d, J = 9.2 Hz, | |
| | (3-chloro-4-cyano-phenoxy)- | 1H), 3.87 (s, 3H), 3.78-3.68 (m, 2H), | |
| | 2,2,4,4-tetramethyl- | 3.65-3.53 (m, 6H), 2.91-2.72 (m, 4H), | |
| | cyclobutyl]pyrimidine-5- | 2.09 (s, 2H), 2.02-1.95 (m, 3H), 1.86 | |
| | carboxamide | (br d, J = 6.4 Hz, 6H), 1.75 (br d, J = | |
| | | 12.0 Hz, 2H), 1.64-1.52 (m, 2H), 1.21 | |
| | | (s, 6H), 1.15-1.07 (m, 6H) | |
| 408 | 2-[8-[5-acetyl-3-[7- | δ = 8.75 (d, J = 4.4 Hz, 2H), 8.21-8.18 | 946.7 |
| | (difluoromethyl)-6-(1- | (m, 1H), 7.90 (d, J = 8.8 Hz, 1H), 7.75 (s, | |
| | methylpyrazol-4-yl)-3,4- | 1H), 7.71 (d, J = 9.2 Hz, 1H), 7.50 (s, | |
| | dihydro-2H-quinolin-1-yl]-6,7- | 1H), 7.21 (d, J = 2.4 Hz, 1H), 7.10 (s, | |
| | dihydro-4H-pyrazolo[4,3- | 1H), 7.00 (m, 1H), 6.95-6.63 (m, 2H), | |
| | c]pyridin-1-yl]-2- | 4.29 (s, 1H), 4.21-4.11 (m, 2H), 4.04 (d, | |
| | azaspiro[4.5]decan-2-yl]-N-[3- | J = 9.2 Hz, 2H), 3.87 (s, 3H), 3.78-3.66 | |
| | (3-chloro-4-cyano-phenoxy)- | (m, 2H), 3.66-3.55 (m, 4H), 3.39 (s, | |
| | 2,2,4,4-tetramethyl- | 2H), 2.90-2.70 (m, 4H), 2.08 (s, 2H), | |
| | cyclobutyl]pyrimidine-5- | 2.03-1.90 (m, 7H), 1.88-1.78 (m, 2H), | |
| | carboxamide | 1.77-1.68 (m, 2H), 1.66-1.54 (m, 2H), | |
| | | 1.21 (s, 6H), 1.11 (s, 6H) | |

### Example 62: Synthesis of 2-[9-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[5.5]undecan-2-yl]-N-[3-(3-chloro-4-cyano-phenoxy)-2,2,4,4-tetramethyl-cyclobutyl]pyrimidine-5-carboxamide (Compound 412)

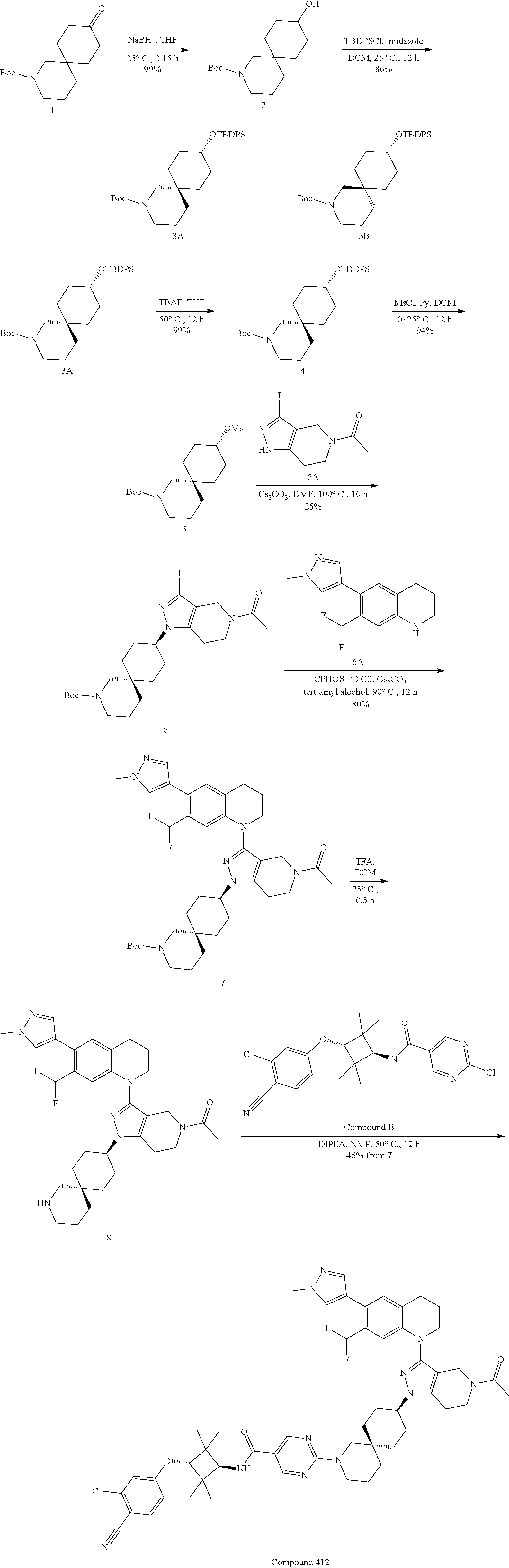

tert-butyl 9-oxo-2-azaspiro[5.5]undecane-2-carboxylate (1 g, 3.74 mmol, 1 eq) in THF (10 mL) was added NaBH₄ (71 mg, 1.87 mmol, 0.5 eq) in THF (4 mL) and EtOH (6 mL). The reaction was stirred at 25° C. for 0.15 h. The reaction mixture was quenched by addition water (30 mL) at 0° C., and then diluted with water (30 mL) and extracted with ethyl acetate (30 mL×3). The combined organic layers were washed with brine (30 mL×3), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜30% Ethylacetate/Petroleum ethergradient @80 mL/min). The desired compound tert-butyl 9-hydroxy-2-azaspiro[5.5]undecane-2-carboxylate (1 g, 3.71 mmol, 99% yield) was obtained as colorless oil (HNMR EW48069-636-P1A).

¹H NMR (400 MHz, CDCl₃) δ=3.74 (tt, J=4.0, 8.0 Hz, 1H), 3.80-3.54 (m, 1H), 3.36 (s, 2H), 3.28 (s, 1H), 3.13 (s, 1H), 1.84-1.73 (m, 2H), 1.66-1.51 (m, 6H), 1.46 (d, J=5.4 Hz, 10H), 1.37-1.31 (m, 1H), 1.28-1.07 (m, 2H)

To a solution of tert-butyl 9-hydroxy-2-azaspiro[5.5]undecane-2-carboxylate (950 mg, 3.53 mmol, 1 eq) in DCM (10 mL) was added imidazole (600 mg, 8.82 mmol, 2.5 eq) and TBDPSCl (1.45 g, 5.29 mmol, 1.35 mL, 1.5 eq). The reaction mixture was stirred at 25° C. for 12 h. The reaction mixture was concentrated under reduced pressure to give a residue. The residue was purified by prep-HPLC (FA condition: column: Phenomenex luna C18 150*40 mm*15 um; mobile phase: [water(FA)-ACN]; gradient: 95%-100% B over 15 min). The desired compound tert-butyl 9-[tert-butyl(diphenyl)silyl]oxy-2-azaspiro[5.5]undecane-2-carboxylate (780 mg, 1.53 mmol, 86% yield) was obtained as yellow oil. The desired compound tert-butyl 9-[tert-butyl(diphenyl)silyl]oxy-2-azaspiro[5.5]undecane-2-carboxylate (920 mg, 1.81 mmol, 51% yield) was obtained as red oil.

¹H NMR (400 MHz, CDCl₃) δ=7.68 (dd, J=1.6, 8.0 Hz, 4H), 7.49-7.31 (m, 6H), 3.73-3.55 (m, 1H), 3.34 (s, 2H), 3.26 (s, 2H), 1.62 (s, 5H), 1.54 (d, J=4.4 Hz, 2H), 1.50 (s, 11H), 1.06 (s, 9H), 1.00-0.90 (m, 2H)

LC-MS: MS (ESI⁺): t_{R}=0.796 min, m/z=508.4 [M+H⁺]

To a solution of tert-butyl 9-[tert-butyl(diphenyl)silyl]oxy-2-azaspiro[5.5]undecane-2-carboxylate (780 mg, 1.54 mmol, 1 eq) in THF (10 mL) was added TBAF (1 M, 3.07 mL, 2 eq). The reaction mixture was stirred at 50° C. for 12 h. The reaction mixture was concentrated under reduced pressure to give a residue. The residue was purified by flash silica gel chromatography (ISCO®; 12 g SepaFlash® Silica Flash Column, Eluent of 0˜30% Ethylacetate/Petroleum ethergradient @80 mL/min). Compound tert-butyl 9-hydroxy-2-azaspiro[5.5]undecane-2-carboxylate (410 mg, 1.52 mmol, 99% yield) was obtained as colorless oil.

¹H NMR (400 MHz, CDCl₃) δ=3.62 (d, J=0.8 Hz, 1H), 3.36 (s, 2H), 3.27 (s, 2H), 1.86-1.74 (m, 2H), 1.57-1.42 (m, 14H), 1.39-1.30 (m, 3H), 1.19-1.09 (m, 2H)

To a solution of tert-butyl 9-hydroxy-2-azaspiro[5.5]undecane-2-carboxylate (410 mg, 1.52 mmol, 1 eq) and Py (392 mg, 4.96 mmol, 0.4 mL, 3.26 eq) in DCM (10 mL) was added methylsulfonyl methanesulfonate (400 mg, 2.30 mmol, 1.51 eq) at 0° C. The reaction mixture was stirred at 25° C. for 12 h. The reaction mixture was concentrated under reduced pressure to give a residue. The residue was purified by flash silica gel chromatography (ISCO®; 20 g SepaFlash® Silica Flash Column, Eluent of 0˜30% Ethylacetate/Petroleum ethergradient @100 mL/min). Compound tert-butyl 9-methylsulfonyloxy-2-azaspiro[5.5]undecane-2-carboxylate (500 mg, 1.44 mmol, 94% yield) was obtained as colorless oil.

¹H NMR (400 MHz, CDCl₃) δ=4.78-4.60 (m, 1H), 3.37 (s, 2H), 3.27 (s, 2H), 3.02 (s, 3H), 2.00-1.77 (m, 4H), 1.74-1.63 (m, 2H), 1.53 (s, 2H), 1.47 (s, 9H), 1.40-1.34 (m, 2H), 1.29-1.17 (m, 2H)

To a solution of tert-butyl 9-methylsulfonyloxy-2-azaspiro[5.5]undecane-2-carboxylate (500 mg, 1.44 mmol, 1 eq) and 1-(3-iodo-1,4,6,7-tetrahydropyrazolo[4,3-c]pyridin-5-yl)ethanone (419 mg, 1.44 mmol, 1 eq) in DMF (5 mL) was added Cs₂CO₃ (1.41 g, 4.32 mmol, 3 eq). The reaction mixture was stirred at 110° C. for 12 h. The reaction mixture was quenched by addition water (30 mL) at 0° C., and then diluted with water (30 mL) and extracted with ethyl acetate (30 mL×3). The combined organic layers were washed with brine (30 mL×3), dried over anhydrous sodium sulfate, filtered and concentrated under reduced pressure to give a residue. The residue was purified by flash silica gel chromatography (ISCO®; 20 g SepaFlash® Silica Flash Column, Eluent of 0-100% Ethylacetate/Petroleum ethergradient @80 mL/min). The residue was purified by prep-HPLC (neutral condition: column: Welch Ultimate XB—CN 250*50*10 um; mobile phase: [Hexane-EtOH]; gradient: 1%-35% B over 15 min. Compound tert-butyl 9-(5-acetyl-3-iodo-6,7-dihydro-4H-pyrazolo[4,3-c]pyridin-1-yl)-2-azaspiro[5.5]undecane-2-carboxylate (200 mg, 368.70 μmol, 25% yield) was obtained as a white solid.

¹H NMR (400 MHz, CDCl₃) δ=4.41 (s, 1H), 4.25 (s, 1H), 3.89 (t, J=5.6 Hz, 2H), 3.71 (t, J=5.6 Hz, 1H), 3.38 (s, 2H), 3.11 (s, 2H), 2.79-2.60 (m, 2H), 2.24-2.17 (m, 3H), 2.15-2.00 (m, 2H), 1.74 (t, J=9.2 Hz, 4H), 1.69-1.64 (m, 2H), 1.53 (s, 2H), 1.46 (s, 9H), 1.33-1.18 (m, 2H)

LC-MS: MS (ESI⁺): t_{R}=0.573 min, m/z=543.2 [M+H⁺]

To a solution of 7-(difluoromethyl)-6-(1-methylpyrazol-4-yl)-1,2,3,4-tetrahydroquinoline (131 mg, 497.74 μmol, 1.5 eq) and tert-butyl 9-(5-acetyl-3-iodo-6,7-dihydro-4H-pyrazolo[4,3-c]pyridin-1-yl)-2-azaspiro[5.5]undecane-2-carboxylate (180 mg, 331.83 μmol, 1 eq) in 2-methylbutan-2-ol (10 mL) was added CPHOS PD G₃ (27 mg, 33.18 μmol, 0.1 eq) and Cs₂CO₃ (324 mg, 995.48 μmol, 3 eq). The reaction mixture was stirred at 90° C. for 12 h under N₂. The reaction mixture was quenched by addition water (10 mL), and then diluted with water (30 mL) and extracted with ethyl acetate (30 mL×3). The combined organic layers were washed with brine (30 mL×3), dried over anhydrous sodium sulfate, filtered and concentrated under reduced pressure to give a residue. The residue was purified by prep-TLC (silica, Petroleum ether/Ethyl acetate=1:3). Compound tert-butyl 9-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[5.5]undecane-2-carboxylate (180 mg, 265.56 μmol, 80% yield) was obtained as light yellow oil.

LC-MS: MS (ESI⁺): t_{R}=0.633 min, m/z=678.3 [M+H⁺]

To a solution of tert-butyl 9-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[5.5]undecane-2-carboxylate (180 mg, 265.56 μmol, 1 eq) in DCM (5 mL) was added TFA (91 mg, 796.67 μmol, 59.18 μL, 3 eq). The reaction mixture was stirred at 25° C. for 0.5 h. The reaction mixture was concentrated under reduced pressure to give a residue. Compound 1-[1-(2-azaspiro[5.5]undecan-9-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (184 mg, crude, TFA) was obtained as yellow oil and directly used in the next step without further purification.

LC-MS: MS (ESI⁺): t_{R}=0.435 min, m/z=578.3 [M+H⁺]

To a solution of 1-[1-(2-azaspiro[5.5]undecan-9-yl)-3-[7-(difluoromethyl)-6-(1-methylpyrazol-4-yl)-3,4-dihydro-2H-quinolin-1-yl]-6,7-dihydro-4H-pyrazolo[4,3-c]pyridin-5-yl]ethanone (92 mg) and DIPEA (172 mg, 1.33 mmol, 231.66 μL, 10 eq) in NMP (2 mL) was added 2-chloro-N-[3-(3-chloro-4-cyano-phenoxy)-2,2,4,4-tetramethyl-cyclobutyl]pyrimidine-5-carboxamide (61 mg, 146.30 μmol, 1.1 eq). The reaction mixture was stirred at 50° C. for 12 h. The reaction mixture was quenched by addition water (10 mL) at 25° C., and then diluted with water (20 mL) and extracted with ethyl acetate (30 mL×3). The combined organic layers were washed with brine (30 mL×3), dried over anhydrous sodium sulfate, filtered and concentrated under reduced pressure to give a residue. The residue was purified by prep-TLC (silica, DCM:MeOH=10:1). The residue was purified by prep-HPLC (FA condition:column: Unisil 3-100 C18 Ultra 150*50 mm*3 um; mobile phase: [water(FA)-ACN]; gradient: 65%-85% B over 10 min). Compound 2-[9-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-2-azaspiro[5.5]undecan-2-yl]-N-[3-(3-chloro-4-cyano-phenoxy)-2,2,4,4-tetramethyl-cyclobutyl]pyrimidine-5-carboxamide (60.17 mg, 62.49 μmol, 46% yield over two steps) was obtained as a white solid.

¹H NMR (400 MHz, CDCl₃) δ=8.68 (s, 2H), 7.63-7.50 (m, 2H), 7.40 (d, J=6.8 Hz, 1H), 7.07-6.95 (m, 2H), 6.89 (d, J=8.8 Hz, 1H), 6.81 (dd, J=2.4, 8.8 Hz, 1H), 6.51 (dt, J=11.2, 55.6 Hz, 1H), 5.93 (d, J=8.0 Hz, 1H), 4.29-4.09 (m, 3H), 4.04 (s, 1H), 3.95 (d, J=2.4 Hz, 3H), 3.92-3.82 (m, 4H), 3.80-3.63 (m, 5H), 2.86 (td, J=6.0, 11.6 Hz, 2H), 2.80-2.67 (m, 2H), 2.20-2.01 (m, 7H), 1.83-1.70 (m, 8H), 1.38 (t, J=13.2 Hz, 2H), 1.24 (d, J=14.8 Hz, 12H)

LC-MS: MS (ESI⁺): t_{R}=3.289 min, m/z=960.6 [M+H⁺]

The compounds below were prepared in a similar manner as described in Example 62.

| | | | LC-MS |
|---|---|---|---|
| Compound | Name | ¹H NMR (CDCl₃) | [M + H⁺] |
| 414 | 2-[3-[9-[5-acetyl-3-[7- | δ = 8.76 (s, 2H), 7.91 (d, J = 8.8 Hz, 1H), | 1015.6 |
| | (difluoromethyl)-6-(1- | 7.77-7.75 (m, 2H), 7.50 (s, 1H), 7.22 (d, | |
| | methylpyrazol-4-yl)-3,4- | J = 2.0 Hz, 1H), 7.10 (s, 1H), 6.92 (dd, | |
| | dihydro-2H-quinolin-1-yl]-6,7- | J = 2.4, 8.8 Hz, 1H), 6.85-6.64 (m, 2H), | |
| | dihydro-4H-pyrazolo[4,3- | 4.30 (s, 1H), 4.18-4.14 (m, 4H), 4.06- | |
| | c]pyridin-1-yl]-2- | 4.01 (m, 2H), 3.89-3.85 (m, 5H), 3.77- | |
| | azaspiro[5.5]undecan-2- | 3.67 (m, 2H), 3.60-3.57 (m, 2H), 3.20- | |
| | yl]azetidin-1-yl]-N-[3-(3- | 3.10 (m, 1H), 2.90-2.80 (m, 3H), 2.75- | |
| | chloro-4-cyano-phenoxy)- | 2.65 (m, 1H), 2.60-2.50 (m, 1H), 2.35- | |
| | 2,2,4,4-tetramethyl- | 2.20 (m, 1H), 2.08 (s, 2H), 2.05-1.90 (m, | |
| | cyclobutyl]pyrimidine-5- | 7H), 1.85-1.65 (m, 4H), 1.60-1.40 (m, | |
| | carboxamide | 4H), 1.35-1.25 (m, 2H), 1.22 (s, 6H), | |
| | | 1.12 (s, 6H) | |
| 415 | 4-[3-[9-[5-acetyl-3-[7- | δ = 7.76-7.64 (m, 4H), 7.52-7.40 (m, | 1009.7 |
| | (difluoromethyl)-6-(1- | 2H), 7.13 (s, 1H), 6.90-6.64 (m, 3H), | |
| | methylpyrazol-4-yl)-3,4- | 6.58-6.38 (m, 3H), 4.26 (s, 1H), 4.25- | |
| | dihydro-2H-quinolin-1-yl]-6,7- | 4.10 (m, 2H), 4.09-3.95 (m, 4H), 3.99 (s, | |
| | dihydro-4Hpyrazolo[4,3- | 3H), 3.91 (s, 3H), 3.75-3.65 (m, 2H), | |
| | c]pyridin-1-yl]-2- | 3.60 (s, 4H), 3.26 (s, 1H), 2.87 (s, 3H), | |
| | azaspiro[5.5]undecan-2- | 2.85 (s, 1H), 2.32-2.20 (m, 2H), 2.08 (s, | |
| | yl]azetidin-1-yl]-N-[3-(4- | 2H), 1.97-1.89 (m, 4H), 1.85-1.40 (m, | |
| | cyano-3-methoxy-phenoxy)- | 9H), 1.24 (s, 9H), 1.14 (s, 6H) | |
| | 2,2,4,4-tetramethyl- | | |
| | cyclobutyl]benzamide | | |
| 416 | 4-[3-[9-[5-acetyl-3-[7- | δ = 7.76-7.74 (m, 3H), 7.65 (d, J = 8.8 Hz, | 1009.7 |
| | (difluoromethyl)-6-(1- | 1H), 7.49-7.48 (m, 2H), 7.10 (s, 1H), | |
| | methylpyrazol-4-yl)-3,4- | 6.83-6.75 (m, 2H), 6.64 (s, 1H), 6.55- | |
| | dihydro-2H-quinolin-1-yl]-6,7- | 6.53 (dd, J₁ = 8.8 Hz; J₂ = 2.4 Hz, 2H), | |
| | dihydro-4Hpyrazolo[4,3- | 6.44 (d, J = 8.4 Hz, 1H), 4.14-4.12 (m, | |
| | c]pyridin-1-yl]-2- | 2H), 4.07-4.05 (m, 4H), 3.98 (s, 3H), | |
| | azaspiro[5.5]undecan-2- | 3.92 (s, 3H), 3.75-3.70 (m, 2H), 3.70- | |
| | yl]azetidin-1-yl]-N-[3-(4- | 3.65 (m, 4H), 3.15-3.10 (m, 1H), 2.80- | |
| | cyano-3-methoxy-phenoxy)- | 2.75 (m, 3H), 2.74-2.70 (m, 1H), 2.25- | |
| | 2,2,4,4-tetramethyl- | 2.23 (m, 2H), 2.10-2.05 (m, 2H), 1.95- | |
| | cyclobutyl]benzamide | 1.90 (m, 7H), 1.80-1.70 (m, 4H), 1.60- | |
| | | 1.45 (m, 4H), 1.23 (s, 8H), 1.15 (s, 6H) | |

### Example 63: Synthesis of 2-[4-[[4-[5-acetyl-3-[7-(difluoromethyl)-6-thiazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (Compound 422)

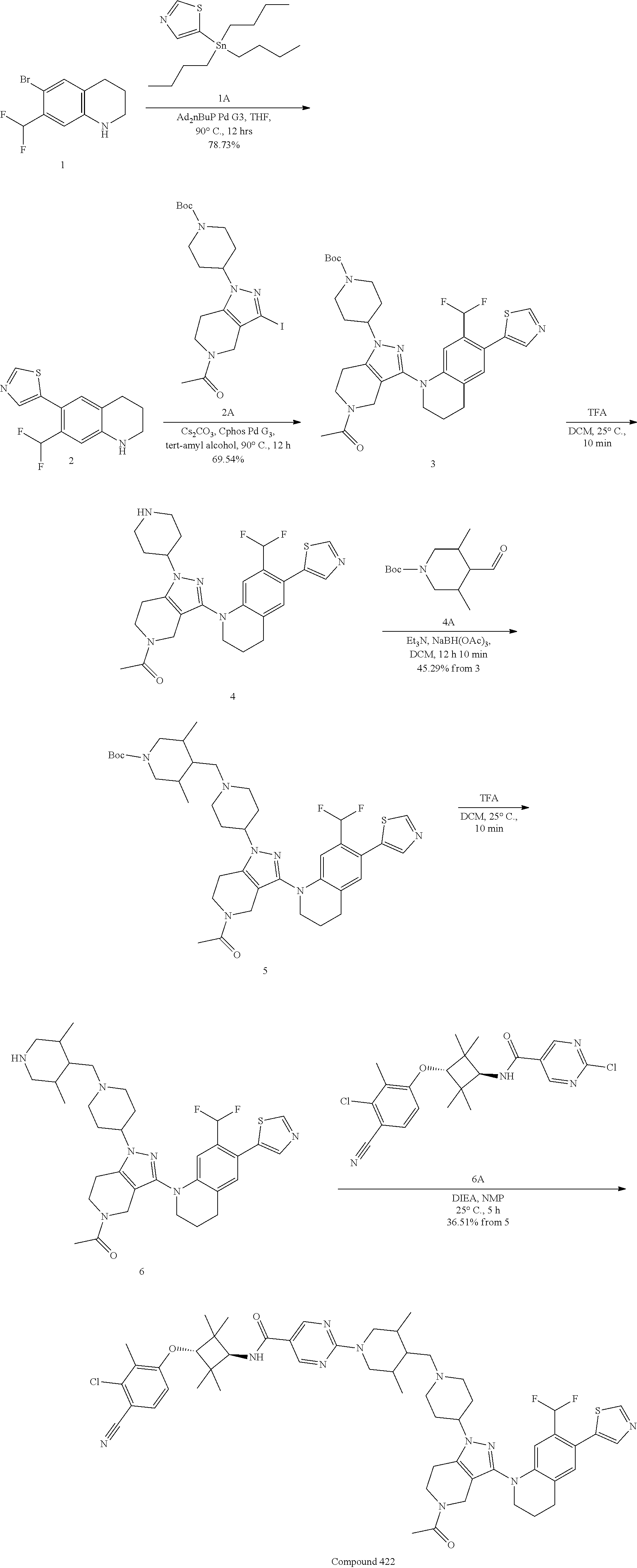

A mixture of 6-bromo-7-(difluoromethyl)-1,2,3,4-tetrahydroquinoline (2.00 g, 7.63 mmol, 1.0 eq), tributyl(thiazol-5-yl)stannane (3.14 g, 8.39 mmol, 1.1 eq), Ad₂nBuP Pd G3 (cataCXium® A Pd G3) (556 mg, 763.09 μmol, 0.1 eq) in THF (30 mL) was degassed and purged with N₂ for 3 times, and then the mixture was stirred at 90° C. for 12 hr under N₂ atmosphere. The reaction mixture was quenched by water (100 mL) and extracted with ethyl acetate (60 mL×3). The combined organic layers were washed with brine (150 mL×2), dried over Na₂SO₄, filtered and concentrated under reduced pressure to give a residue. The residue was purified by flash silica gel chromatography (SiO₂, Petroleum ether/Ethyl acetate=10/1 to 3/1). 5-[7-(difluoromethyl)-1,2,3,4-tetrahydroquinolin-6-yl]thiazole (1.60 g, 6.01 mmol, 78.73% yield) was obtained as a yellow oil.

LC-MS: MS (ESI⁺): t_{R}=0.503 min, m/z=266.9 [M+H⁺]

A mixture of 5-[7-(difluoromethyl)-1,2,3,4-tetrahydroquinolin-6-yl]thiazole (750 mg, 2.82 mmol, 1.2 eq), tert-butyl 4-(5-acetyl-3-iodo-6,7-dihydro-4H-pyrazolo[4,3-c]pyridin-1-yl)piperidine-1-carboxylate (1.11 g, 2.35 mmol, 1.0 eq), Cs₂CO₃ (2.29 g, 7.04 mmol, 3.0 eq), CPHOS Pd G3 (189 mg, 234.69 μmol, 0.1 eq) in 2-methylbutan-2-ol (8 mL) was degassed and purged with N₂ for 3 times, and then the mixture was stirred at 90° C. for 12 hr under N₂ atmosphere. The reaction mixture was quenched by water (80 mL) and extracted with EtOAc (50 mL×3). The combined organic layers were washed with brine (100 mL×2), dried over Na₂SO₄, filtered and concentrated under reduced pressure to give a residue. The residue was purified by flash silica gel chromatography (SiO₂, Petroleum ether/Tetrahydrofuran=10/1 to 1/1). tert-butyl 4-[5-acetyl-3-[7-(difluoromethyl)-6-thiazol-5-yl-3,4-dihydro-2H-quinolin-1-yl]-6,7-dihydro-4H-pyrazolo[4,3-c]pyridin-1-yl]piperidine-1-carboxylate (1.00 g, 1.63 mmol, 69.54% yield) was obtained as a yellow oil.

LC-MS: MS (ESI⁺): t_{R}=0.560 min, m/z=613.2 [M+H⁺]

To a solution of tert-butyl 4-[5-acetyl-3-[7-(difluoromethyl)-6-thiazol-5-yl-3,4-dihydro-2H-quinolin-1-yl]-6,7-dihydro-4H-pyrazolo[4,3-c]pyridin-1-yl]piperidine-1-carboxylate (150 mg, 244.81 μmol, 1.0 eq) in DCM (2 mL) was added TFA (3.07 g, 26.93 mmol, 2 mL, 109.99 eq). The mixture was stirred at 25° C. for 10 min. The reaction mixture was concentrated under reduced pressure to give a residue. 1-[3-[7-(difluoromethyl)-6-thiazol-5-yl-3, 4-dihydro-2H-quinolin-1-yl]-1-(4-piperidyl)-6, 7-dihydro-4H-pyrazolo[4,3-c]pyridin-5-yl]ethanone (150 mg) was obtained as a yellow oil and used into the next step without further purification.

LC-MS: MS (ESI⁺): t_{R}=0.419 min, m/z=513.1 [M+H⁺]

To a solution of 1-[3-[7-(difluoromethyl)-6-thiazol-5-yl-3,4-dihydro-2H-quinolin-1-yl]-1-(4-piperidyl)-6,7-dihydro-4H-pyrazolo[4,3-c]pyridin-5-yl]ethanone (150 mg) in DCM (5 mL) was added Et₃N (73 mg, 718.12 μmol, 99.95 μL, 3.0 eq) and tert-butyl 4-formyl-3,5-dimethyl-piperidine-1-carboxylate (87 mg, 359.06 μmol, 1.5 eq), then the mixture was stirred at 25° C. for 10 min. Then NaBH(OAc)₃ (254 mg, 1.20 mmol, 5.0 eq) was added to the reaction mixture at 25° C. and it was stirred at 25° C. for 12 h. The reaction mixture was quenched by water (20 mL) and extracted with DCM (10 mL×3). The combined organic layers were washed with brine (50 mL×2), dried over Na₂SO₄, filtered and concentrated under reduced pressure to give a residue. The residue was purified by prep-TLC (SiO₂, Dichloromethane:Methanol=15:1). tert-butyl 4-[[4-[5-acetyl-3-[7-(difluoromethyl)-6-thiazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-piperidine-1-carboxylate (80 mg, 108.41 μmol, 45.29% yield over two steps) was obtained as a yellow solid.

LC-MS: MS (ESI⁺): t_{R}=0.485 min, m/z=738.3 [M+H⁺]

To a solution of tert-butyl 4-[[4-[5-acetyl-3-[7-(difluoromethyl)-6-thiazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-piperidine-1-carboxylate (80 mg, 108.41 mol, 1.0 eq) in DCM (2 mL) was added TFA (3.07 g, 26.93 mmol, 2 mL, 248.36 eq). The mixture was stirred at 25° C. for 10 min. The reaction mixture was concentrated under reduced pressure to give a residue. 1-[3-[7-(difluoromethyl)-6-thiazol-5-yl-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (80 mg) was obtained as a yellow oil and used into the next step without further purification.

To a solution of 1-[3-[7-(difluoromethyl)-6-thiazol-5-yl-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (80 mg) in NMP (3 mL) was added DIPEA (41 mg, 319.21 μmol, 55.60 μL, 3.0 eq). The mixture was added 2-chloro-N-[3-(3-chloro-4-cyano-phenoxy)-2,2,4,4-tetramethyl-cyclobutyl]pyrimidine-5-carboxamide (45 mg, 106.40 μmol, 1.0 eq) and stirred at 25° C. for 5 hr. The reaction mixture was concentrated under reduced pressure to give a residue. The residue was purified by prep-HPLC (column: Phenomenex luna C18 150*25 mm*10 um; mobile phase: [water(FA)-ACN]; gradient: 33%-63% B over 10 min). 2-[4-[[4-[5-acetyl-3-[7-(difluoromethyl)-6-thiazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (39.65 mg, 38.85 μmol, 36.51% yield over two steps) was obtained as a white solid.

¹H NMR (400 MHz, DMSO-d₆) δ=9.16 (s, 1H), 8.78 (s, 2H), 7.92-7.80 (d, 1H), 7.80 (s, 1H), 7.72-7.70 (d, 1H), 7.22-7.18 (d, 2H), 7.02-7.00 (d, 1H), 7.00-6.82 (m, 2H), 4.29 (s, 1H), 4.22-4.17 (d, 2H), 4.04-4.02 (d, 2H), 3.94-3.91 (d, 2H), 3.75-3.65 (m, 2H), 3.62-3.60 (m, 2H), 3.45-3.43 (m, 2H), 3.02-2.97 (m, 2H), 2.75-2.60 (m, 4H), 2.30-2.25 (m, 2H), 2.10-1.90 (m, 9H), 2.00-1.75 (m, 5H), 1.25 (s, 6H), 1.10 (s, 6H), 1.00-0.90 (d, 6H).

LC-MS: MS (ESI⁺): t_{R}=2.555 min, m/z=1020.6 [M+H⁺]

### Example 64: Synthesis of 2-[4-[[4-[5-acetyl-3-[7-(difluoromethyl)-6-oxazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (Compound 424)

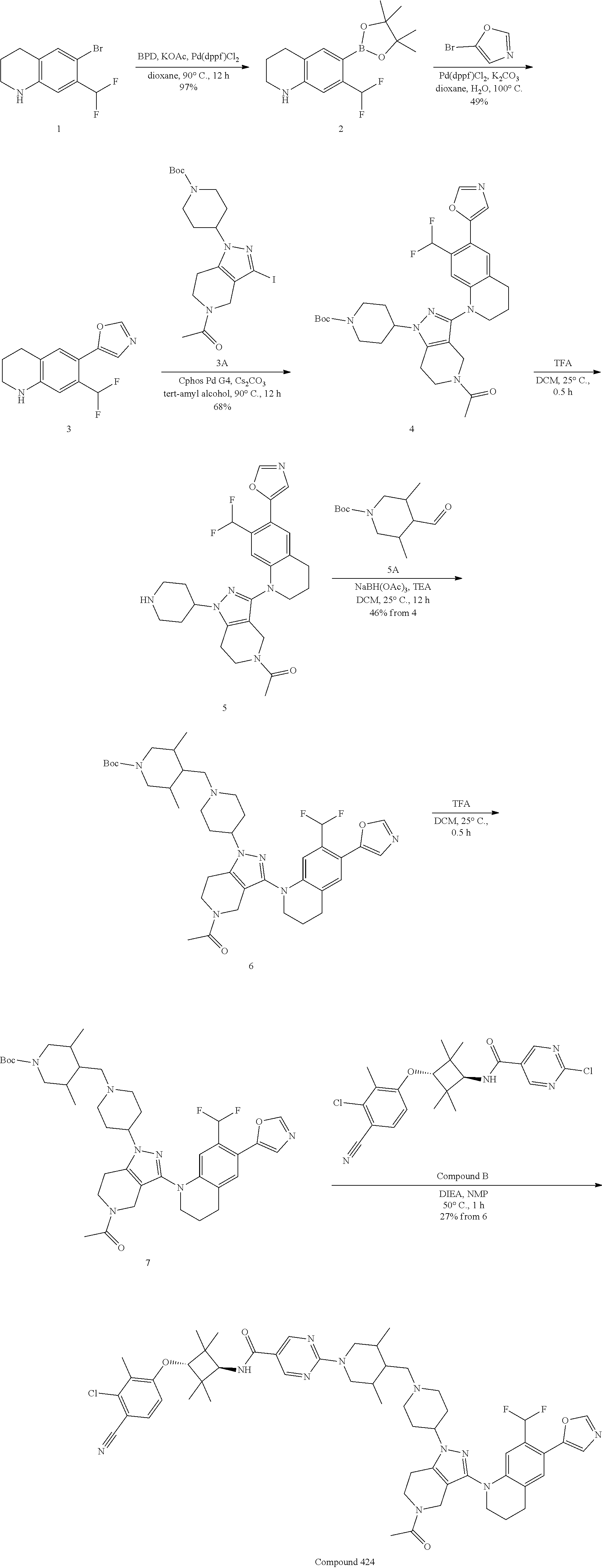

A mixture of 6-bromo-7-(difluoromethyl)-1,2,3,4-tetrahydroquinoline (2.00 g, 7.63 mmol, 1.0 eq), BPD (2.91 g, 11.45 mmol, 1.5 eq), Pd(dppf)Cl₂ (558 mg, 763 μmol, 0.1 eq), KOAc (2.25 g, 22.89 mmol, 3.0 eq) in dioxane (30 mL) was degassed and purged with N₂ for 3 times, and then the mixture was stirred at 90° C. for 12 h under N₂ atmosphere. The reaction mixture was quenched by addition water 30 mL, and then extracted with EtOAc (20 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/0 to 10/1). Compound 7-(difluoromethyl)-6-(4,4,5,5-tetramethyl-1,3,2-dioxaborolan-2-yl)-1,2,3,4-tetrahydroquinoline (2.30 g, 7.44 mmol, 97% yield) was obtained as a yellow solid.

A mixture of 7-(difluoromethyl)-6-(4,4,5,5-tetramethyl-1,3,2-dioxaborolan-2-yl)-1,2,3,4-tetrahydroquinoline (1.88 g, 6.08 mmol, 1.5 eq), 5-bromooxazole (0.60 g, 4.06 mmol, 1.0 eq), Pd(dppf)Cl₂ (297 mg, 405 μmol, 0.1 eq), K₂CO₃ (1.5 M, 8.11 mL, 3.0 eq) in dioxane (20 mL) was degassed and purged with N₂ for 3 times, and then the mixture was stirred at 90° C. for 12 h under N₂ atmosphere. The reaction mixture was quenched by addition water 30 mL, and then extracted with EtOAc (20 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=10/1 to 3/1). Compound 5-[7-(difluoromethyl)-1,2,3,4-tetrahydroquinolin-6-yl]oxazole (500 mg, 2.00 mmol, 49% yield) was obtained as a yellow solid.

¹H NMR (400 MHz, DMSO-d₆) 8.43-8.37 (m, 1H), 7.27-7.22 (m, 2H), 7.22-6.91 (m, 1H), 6.83 (s, 1H), 6.47 (br s, 1H), 3.31-3.25 (m, 2H), 2.79-2.73 (m, 2H), 1.88-1.81 (m, 2H).

LC-MS: MS (ESI⁺: t_{R}=0.460 min, m/z=250.9 [M+H⁺]

A mixture of tert-butyl 4-(5-acetyl-3-iodo-6,7-dihydro-4H-pyrazolo[4,3-c]pyridin-1-yl)piperidine-1-carboxylate (700 mg, 1.48 mmol, 1.0 eq), 5-[7-(difluoromethyl)-1,2,3,4-tetrahydroquinolin-6-yl]oxazole (388 mg, 1.55 mmol, 1.05 eq), Cphos Pd G3 (119 mg, 148 μmol, 0.1 eq), Cs₂CO₃ (1.44 g, 4.43 mmol, 3.0 eq) in 2-methylbutan-2-ol (10 mL) was degassed and purged with N₂ for 3 times, and then the mixture was stirred at 90° C. for 12 h under N₂ atmosphere. The reaction mixture was quenched by addition water 30 mL, and then extracted with EtOAc (20 mL×2). The combined organic layers were washed with brine (50 mL×2), dried over Na₂SO₄, filtered and concentrated under reduced pressure to give a residue. The residue was purified by column chromatography (SiO₂, Petroleum ether/THF=10/1 to 1/2). Compound tert-butyl 4-[5-acetyl-3-[7-(difluoromethyl)-6-oxazol-5-yl-3,4-dihydro-2H-quinolin-1-yl]-6,7-dihydro-4H-pyrazolo[4,3-c]pyridin-1-yl]piperidine-1-carboxylate (600 mg, 1.01 mmol, 68% yield) was obtained as a yellow solid.

LC-MS: MS (ESI: t_{R}=0.532 min, m/z=597.3 [M+H⁺]

To a solution of tert-butyl 4-[5-acetyl-3-[7-(difluoromethyl)-6-oxazol-5-yl-3,4-dihydro-2H-quinolin-1-yl]-6,7-dihydro-4H-pyrazolo[4,3-c]pyridin-1-yl]piperidine-1-carboxylate (150 mg, 251 μmol, 1.0 eq) in DCM (2 mL) was added TFA (2 mL). The mixture was stirred at 25° C. for 0.5 h. The reaction mixture was concentrated under reduced pressure to remove solvent. Compound 1-[3-[7-(difluoromethyl)-6-oxazol-5-yl-3,4-dihydro-2H-quinolin-1-yl]-1-(4-piperidyl)-6,7-dihydro-4H-pyrazolo[4,3-c]pyridin-5-yl]ethanone (120 mg) was obtained as a yellow oil and directly used in the next step without further purification.

LC-MS: MS (ESI⁺: t_{R}=0.394 min, m/z=497.2 [M+H⁺]

To a solution of 1-[3-[7-(difluoromethyl)-6-oxazol-5-yl-3,4-dihydro-2H-quinolin-1-yl]-1-(4-piperidyl)-6,7-dihydro-4H-pyrazolo[4,3-c]pyridin-5-yl]ethanone (120 mg) and tert-butyl 4-formyl-3,5-dimethyl-piperidine-1-carboxylate (79 mg, 326 μmol, 1.35 eq) in DCM (2 mL) was added NaBH(OAc)₃ (256 mg, 1.21 mmol, 5.0 eq) and Et₃N (73 mg, 725 μmol, 101 μL, 3.0 eq). The mixture was stirred at 25° C. for 2 h. The reaction mixture was quenched by addition water 30 mL, and then extracted with DCM (20 mL×2). The combined organic layers were washed with brine (50 mL×2), dried over Na₂SO₄, filtered and concentrated under reduced pressure to give a residue. The residue was purified by prep-TLC (SiO₂, DCM:MeOH=10:1). Compound tert-butyl 4-[[4-[5-acetyl-3-[7-(difluoromethyl)-6-oxazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-piperidine-1-carboxylate (80 mg, 111 μmol, 46% yield over two steps) was obtained as a yellow solid.

LC-MS: MS (ESI⁺): t_{R}=0.481 min, m/z=722.4 [M+H⁺]

To a solution of tert-butyl 4-[[4-[5-acetyl-3-[7-(difluoromethyl)-6-oxazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-piperidine-1-carboxylate (80 mg, 111 μmol, 1.0 eq) in DCM (1 mL) was added TFA (1 mL). The mixture was stirred at 25° C. for 0.5 h. The reaction mixture was concentrated under reduced pressure to remove solvent. Compound 1-[3-[7-(difluoromethyl)-6-oxazol-5-yl-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (65 mg) was obtained as a yellow oil and directly used in the next step without further purification.

LC-MS: MS (ESI⁺): t_{R}=0.399 min, m/z=622.3 [M+H⁺]

A solution of 1-[3-[7-(difluoromethyl)-6-oxazol-5-yl-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (65 mg) in NMP (1 mL) was added DIPEA (68 mg, 523 μmol, 91.05 μL, 5.0 eq) and 2-chloro-N-[3-(3-chloro-4-cyano-phenoxy)-2,2,4,4-tetramethyl-cyclobutyl]pyrimidine-5-carboxamide (44 mg, 105 μmol, 1.0 eq). The mixture was stirred at 25° C. for 12 h. The reaction mixture was quenched by addition water 30 mL, and then extracted with EtOAc (20 mL*2). The combined organic layers were washed with brine (50 mL*2), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 32%-62% B over 10 min). Compound 2-[4-[[4-[5-acetyl-3-[7-(difluoromethyl)-6-oxazol-5-yl-3,4-dihydro-2H-quinolin-1-yl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (28.49 mg, 28.36 μmol, 27.13% yield over two steps) was obtained as an off-white solid.

¹H NMR (400 MHz, CDCl₃) δ=8.77-8.70 (m, 2H), 8.47-8.40 (m, 1H), 8.30-8.28 (m, 1H), 7.90 (d, J=8.8 Hz, 1H), 7.70 (br d, J=9.3 Hz, 1H), 7.44-7.40 (m, 1H), 7.30 (s, 1H), 7.22 (d, J=2.1 Hz, 1H), 7.01 (dd, J=2.3, 8.8 Hz, 2H), 6.91-6.86 (m, 1H), 4.29 (s, 1H), 4.24-4.15 (m, 2H), 4.03 (br d, J=9.3 Hz, 2H), 3.97-3.90 (m, 2H), 3.79-3.68 (m, 2H), 3.67-3.59 (m, 2H), 3.47 (br dd, J=8.7, 11.1 Hz, 3H), 3.03 (br d, J=6.3 Hz, 2H), 2.92-2.72 (m, 4H), 2.36-2.29 (m, 2H), 2.09 (s, 3H), 2.04 (br d, J=9.6 Hz, 3H), 1.98 (br s, 3H), 1.86 (br d, J=9.4 Hz, 5H), 1.22 (s, 6H), 1.11 (s, 6H), 0.98-0.91 (m, 6H)

LC-MS: MS (ESI⁺): t_{R}=2.511 min, m/z=1004.7 [M+H⁺]

### Example 65: Synthesis of 2-[4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (Compound 440)

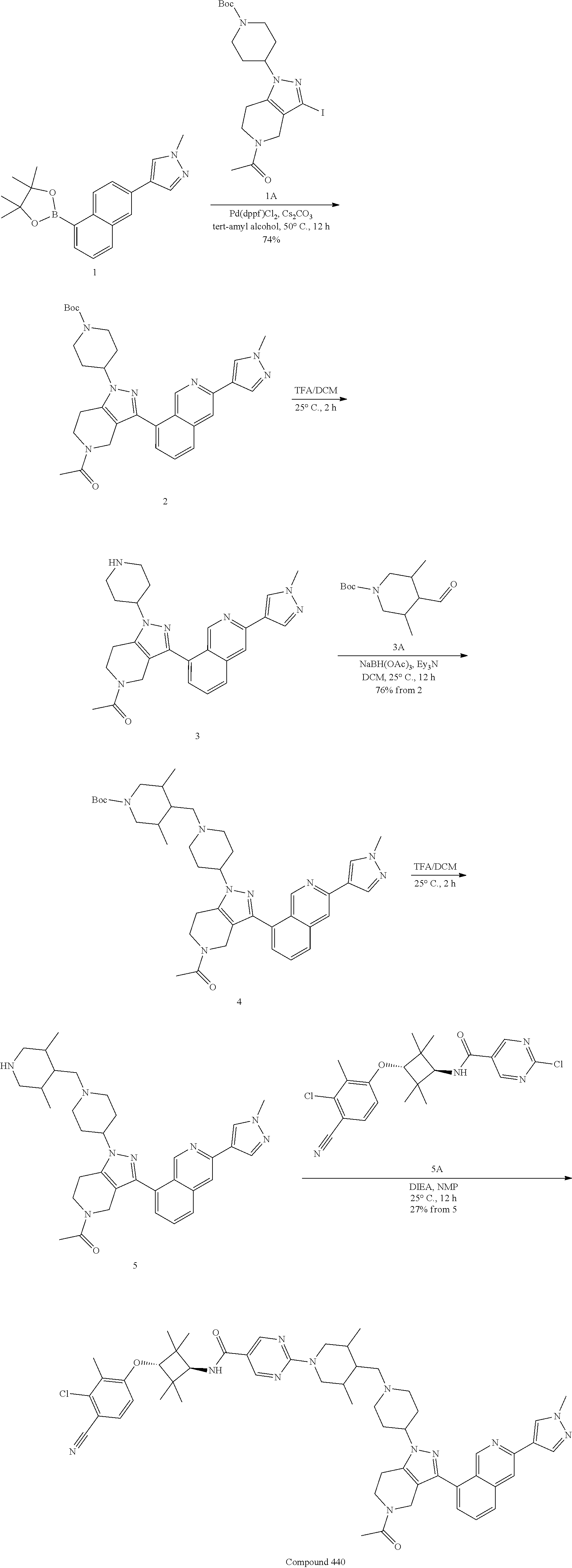

To a solution of 3-(1-methylpyrazol-4-yl)-8-(4,4,5,5-tetramethyl-1,3,2-dioxaborolan-2-yl)isoquinoline (650 mg, 2 mmol, 1.0 eq) and tert-butyl 4-(5-acetyl-3-iodo-6,7-dihydro-4H-pyrazolo[4,3-c]pyridin-1-yl)piperidine-1-carboxylate (920 mg, 2 mmol, 1.0 eq) in THF (20 mL) and H₂O (5 mL) was added Cs₂CO₃ (1.90 g, 6 mmol, 3.0 eq), the mixture was purged with N₂ for 3 times and then Pd(dppf)Cl₂ (142 mg, 194 μmol, 0.1 eq) was added into the mixture. The mixture was stirred at 50° C. for 12 h and the mixture was diluted with water (20 mL), extracted with EtOAc (30 mL×2), washed with brine (20 mL), dried over Na₂SO₄, filtered and then concentrated. Then the residue was purified by column chromatography (SiO₂, Petroleum ether:THF=3:1 to 0:1). Compound tert-butyl 4-[5-acetyl-3-[3-(1-methylpyrazol-4-yl)-8-isoquinolyl]-6,7-dihydro-4H-pyrazolo[4,3-c]pyridin-1-yl]piperidine-1-carboxylate (800 mg, 1 mmol, 74% yield) as a yellow solid.

LC-MS: MS (ESI⁺): t_{R}=0.477 min, m/z=556.4 [M+H⁺]

To a solution of tert-butyl 4-[5-acetyl-3-[3-(1-methylpyrazol-4-yl)-8-isoquinolyl]-6,7-dihydro-4H-pyrazolo[4,3-c]pyridin-1-yl]piperidine-1-carboxylate (470 mg, 846 μmol, 1.0 eq) in DCM (5 mL) was added TFA (4.50 g, 40 mmol, 3 mL, 56.1 eq). The mixture was stirred at 25° C. for 2 h and the reaction mixture was concentrated under reduced pressure to give a residue. Compound 1-[3-[3-(1-methylpyrazol-4-yl)-8-isoquinolyl]-1-(4-piperidyl)-6,7-dihydro-4H-pyrazolo[4,3-c]pyridin-5-yl]ethanone (500 mg) was obtained as a yellow oil and directly used in the next step without further purification.

LC-MS: MS (ESI⁺): t_{R}=0.452 min, m/z=456.1 [M+H⁺]

To a mixture of 1-[3-[3-(1-methylpyrazol-4-yl)-8-isoquinolyl]-1-(4-piperidyl)-6,7-dihydro-4H-pyrazolo[4,3-c]pyridin-5-yl]ethanone (450 mg) and tert-butyl 4-formyl-3,5-dimethyl-piperidine-1-carboxylate (358 mg, 2 mmol, 1.5 eq) in DCM (6 mL) was added Et₃N (500 mg, 5 mmol, 687 μL, 5.0 eq) and NaBH(OAc)₃ (628 mg, 3 mmol, 3.0 eq), the mixture was stirred at 25° C. for 12 h and then the reaction mixture was concentrated under reduced pressure to give a residue. Then the residue was purified by prep-TLC (Dichloromethane:Methanol=20:1). Compound tert-butyl 4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-3,5-dimethyl-piperidine-1-carboxylate (517 mg, 753 μmol, 76% yield from two steps) was obtained as a yellow oil.

LC-MS: MS (ESI⁺): t_{R}=0.503 min, m/z=681.4 [M+H⁺]

To a solution of tert-butyl 4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-3,5-dimethyl-piperidine-1-carboxylate (517 mg, 759 μmol, 1.0 eq) in DCM (5.2 mL) was added TFA (4.00 g, 35 mmol, 2.6 mL, 55.3 eq) then the mixture was stirred at 25° C. for 2 h and then the reaction mixture was concentrated to give the residue. Compound 1-[1-[1-[(3,5-dimethyl-4-piperidyl) methyl]-4-piperidyl]-3-[3-(1-methylpyrazol-4-yl)-8-isoquinolyl]-6,7-dihydro-4H-pyrazolo[4,3-c]pyridin-5-yl]ethanone (460 mg) was obtained as a yellow oil and directly used in the next step without further purification.

LC-MS: MS (ESI⁺): t_{R}=0.455 min, m/z=581.4 [M+H⁺]

To a solution of 1-[1-[1-[(3,5-dimethyl-4-piperidyl)methyl]-4-piperidyl]-3-[3-(1-methylpyrazol-4-yl)-8-isoquinolyl]-6,7-dihydro-4H-pyrazolo[4,3-c]pyridin-5-yl]ethanone (50 mg) and 2-chloro-N-[3-(3-chloro-4-cyano-phenoxy)-2,2,4,4-tetramethyl-cyclobutyl]pyrimidine-5-carboxamide (36 mg, 86 μmol, 1.0 eq) in NMP (1 mL) was added DIPEA (56 mg, 430 μmol, 75 μL, 5.0 eq). The mixture was stirred at 25° C. for 12 h and then the mixture was poured into water (20 mL). The aqueous phase was extracted with EtOAc (20 mL×2). The combined organic phase was washed with brine (20 mL×3), dried with anhydrous sodium sulfate, filtered and concentrated in vacuum. Then the residue was purified by prep-HPLC (column: Phenomenex Luna C18 150*25 mm*10 um; mobile phase: [H₂O (0.225% FA)-ACN]; gradient: 21%-51% B over 10.0 min). Compound 2-[4-[[4-[5-acetyl-3-[3-(1-methylpyrazol-4-yl)-8-isoquinolyl]-6,7-dihydro-4H-pyrazolo[4,3-c]pyridin-1-yl]-1-piperidyl]methyl]-3,5-dimethyl-1-piperidyl]-N-[3-(3-chloro-4-cyano-phenoxy)-2,2,4,4-tetramethyl-cyclobutyl]pyrimidine-5-carboxamide (22.54 mg, 23.33 μmol, 27% yield) was obtained as an off-white solid.

¹H NMR (400 MHz, DMSO-d₆) δ=9.81 (s, 1H), 8.73 (s, 2H), 8.36 (s, 1H), 8.14 (s, 1H), 8.09 (s, 2H), 7.92-7.86 (m, 2H), 7.80 (m, J=7.22 Hz, 1H), 7.70 (d, J=9.2 Hz, 1H), 7.60-7.49 (m, 1H), 7.21 (d, J=2.4 Hz, 1H), 7.00 (dd, J=2.4, 8.8 Hz, 1H), 4.56 (m, 2H), 4.28 (s, 1H), 4.22-4.13 (m, 1H), 4.02 (d, J=9.2 Hz, 1H), 3.93-3.78 (m, 7H), 3.58-3.50 (8, 2H), 3.11 (m, 2H), 2.95 (m, 1H), 2.90-2.72 (m, 1H), 2.36 (m, 2H), 2.11 (s, 5H), 2.03-1.96 (m, 3H), 1.95-1.84 (m, 3H), 1.60-1.53 (d, 1H), 1.21 (s, 6H), 1.10 (s, 6H), 0.95 (in, J=7.0 Hz, 6H)

LC-MS: MS (ESI⁺): t_{R}=1.475 min, m/z=963.8 [M+H⁺].

SFC: t_{R}=2.831 min

The compounds below were prepared in a similar manner as described in Example 65.

| | | | LC-MS |
|---|---|---|---|
| Compound | Name | ¹H NMR (DMSO-d6) | [M + H⁺] |
| 441 | 4-[4-[[4-[5-acetyl-3-[3-(1- | δ = 9.80 (s, 1H), 8.36 (s, 1H), 8.09 (s, | 957.9 |
| | methylpyrazol-4-yl)-8- | 2H), 7.91-7.85 (m, 1H), 7.82-7.76 (m, | |
| | isoquinolyl]-6,7-dihydro-4H- | 1H), 7.72 (d, J = 8.8 Hz, 2H), 7.64 (d, J = | |
| | pyrazolo[4,3-c]pyridin-1-yl]-1- | 8.8 Hz, 1H), 7.60-7.44 (m, 2H), 6.94 (d, | |
| | piperidyl]methyl]-3,5-dimethyl- | J = 8.8 Hz, 2H), 6.63 (d, J = 2.0 Hz, 1H), | |
| | 1-piperidyl]-N-[3-(4-cyano-3- | 6.53 (d, J = 2.0, 8.6 Hz, 1H), 4.55 (s, | |
| | methoxy-phenoxy)-2,2,4,4- | 2H), 4.27 (s, 1H), 4.22-4.10 (m, 1H), | |
| | tetramethyl- | 4.04 (d, J = 9.2 Hz, 1H), 3.91 (d, J = 3.2 | |
| | cyclobutyl]benzamide | Hz, 6H), 3.87-3.77 (m, 2H), 3.38 (s, | |
| | | 2H), 3.09 (s, 2H), 2.98-2.73 (m, 4H), | |
| | | 2.33-2.25 (m, 2H), 2.15-1.90 (m, | |
| | | 11H), 1.86-1.77 (m, 1H), 1.22 (s, 6H), | |
| | | 1.14 (s, 6H), 1.02 (d, J = 6.8 Hz, 6H) | |
| 425 | 2-[4-[[4-[5-acetyl-3-[7- | δ = 8.73 (s, 2H), 7.95-7.85 (d, 1H), 7.75- | 1017.8 |
| | (difluoromethyl)-6-(1- | 7.55 (m, 3H), 7.35 (s, 2H), 7.25 (s, 1H), | |
| | methylimidazol-4-yl)-3,4- | 7.05-6.95 (d, 1H), 6.80 (s, 1H), 4.35 (s, | |
| | dihydro-2H-quinolin-1-yl]-6,7- | 1H), 4.25-4.15 (m, 2H), 4.10-4.00 (m, | |
| | dihydro-4H-pyrazolo[4,3- | 2H), 4.00-3.90 (m, 2H), 3.80-3.55 (m, | |
| | c]pyridin-1-yl]-1- | 8H), 3.10-3.00 (m, 2H), 2.95-2.80 (m, | |
| | piperidyl]methyl]-3,5-dimethyl- | 4H), 2.80-2.65 (m, 1H), 2.35-2.30 (m, | |
| | 1-piperidyl]-N-[3-(3-chloro-4- | 2H), 2.10-1.95 (m, 9H), 1.95-1.80 (m, | |
| | cyano-phenoxy)-2,2,4,4- | 5H), 1.30 (s, 6H), 1.12 (s, 6H), 1.00- | |
| | tetramethyl- | 0.90 (d, 6H) | |
| | cyclobutyl]pyrimidine-5- | | |
| | carboxamide | | |
| 444 | 2-[4-[[4-[5-acetyl-3-[7-methyl- | (CDCl₃) δ = 8.74-8.65 (m, 2H), 7.62- | 981.7 |
| | 6-(1-methylpyrazol-4-yl)-3,4- | 7.51 (m, 2H), 7.41-7.33 (m, 1H), 7.03- | |
| | dihydro-2H-quinolin-1-yl]-6,7- | 6.90 (m, 2H), 6.84-6.77 (m, 1H), 6.42- | |
| | dihydro-4H-pyrazolo[4,3- | 6.33 (m, 1H), 5.93 (d, J = 8.0 Hz, 1H), | |
| | c]pyridin-1-yl]-1- | 4.28 (s, 1H), 4.16-4.10 (m, 2H), 4.09- | |
| | piperidyl]methyl]-3,5-dimethyl- | 3.97 (m, 3H), 3.97-3.87 (m, 5H), 3.78- | |
| | 1-piperidyl]-N-[3-(3-chloro-4- | 3.61 (m, 3H), 3.49-3.29 (m, 2H), 3.18- | |
| | cyano-phenoxy)-2,2,4,4- | 2.98 (m, 2H), 2.89-2.70 (m, 4H), 2.41- | |
| | tetramethyl- | 2.31 (m, 2H), 2.29-2.19 (m, 5H), 2.17 | |
| | cyclobutyl]pyrimidine-5- | (s, 1H), 2.12-2.00 (m, 6H), 1.98-1.83 | |
| | carboxamide | (m, 5H), 1.23 (d, J = 13.6 Hz, 12H), 1.08- | |
| | | 0.99 (m, 6H) | |
| 446 | 2-[4-[[4-[5-acetyl-3-[7-methyl- | (CDCl₃) δ = 8.69 (s, 2H), 7.64-7.50 (m, | 981.7 |
| | 6-(1-methylpyrazol-4-yl)-3,4- | 2H), 7.46-7.33 (m, 1H), 7.09-6.93 (m, | |
| | dihydro-2H-quinolin-1-yl]-6,7- | 2H), 6.82 (dd, J = 2.4, 8.8 Hz, 1H), 6.39 | |
| | dihydro-4H-pyrazolo[4,3- | (s, 1H), 5.97 (br d, J = 8.4 Hz, 1H), 4.89 | |
| | c]pyridin-1-yl]-1- | (br d, J = 12.0 Hz, 1H), 4.53 (br d, J = | |
| | piperidyl]methyl]-3,3-dimethyl- | 12.8 Hz, 1H), 4.30 (s, 1H), 4.17-4.12 | |
| | 1-piperidyl]-N-[3-(3-chloro-4- | (m, 2H), 4.06 (s, 1H), 3.99-3.94 (m, | |
| | cyano-phenoxy)-2,2,4,4- | 3H), 3.92 (br t, J = 5.6 Hz, 1H), 3.76 (br | |
| | tetramethyl- | t, J = 5.6 Hz, 1H), 3.71-3.60 (m, 2H), | |
| | cyclobutyl]pyrimidine-5- | 3.46-3.08 (m, 2H), 2.99-2.68 (m, 7H), | |
| | carboxamide | 2.67-2.58 (m, 1H), 2.41-2.22 (m, 7H), | |
| | | 2.18 (s, 2H), 2.12-1.95 (m, 7H), 1.63- | |
| | | 1.34 (m, 2H), 1.25 (d, J = 15.6 Hz, 12H), | |
| | | 1.07 (s, 3H), 0.80 (s, 3H) | |
| 447 | 4-[4-[[4-[5-acetyl-3-[7-methyl- | (CDCl₃) δ = 7.67 (d, J = 8.8 Hz, 2H), | 975.8 |
| | 6-(1-methylpyrazol-4-yl)-3,4- | 7.54 (d, J = 7.6 Hz, 1H), 7.45 (d, J = 8.8 | |
| | dihydro-2H-quinolin-1-yl]-6,7- | Hz, 1H), 7.40-7.34 (m, 1H), 7.05-6.95 | |
| | dihydro-4H-pyrazolo[4,3- | (m, 1H), 6.86 (d, J = 8.8 Hz, 2H), 6.47 | |
| | c]pyridin-1-yl]-1- | (d, J = 2.0 Hz, 1H), 6.43-6.35 (m, 2H), | |
| | piperidyl]methyl]-3,3-dimethyl- | 6.13 (d, J = 8.0 Hz, 1H), 4.29 (s, 1H), | |
| | 1-piperidyl]-N-[3-(4-cyano-3- | 4.18-4.11 (m, 2H), 4.05 (s, 1H), 3.97- | |
| | methoxy-phenoxy)-2,2,4,4- | 3.88 (m, 7H), 3.84-3.73 (m, 2H), 3.70- | |
| | tetramethyl- | 3.52 (m, 3H), 3.39 (br d, J = 12.8 Hz, | |
| | cyclobutyl]benzamide | 2H), 2.97-2.70 (m, 7H), 2.61 (br d, J = | |
| | | 12.8 Hz, 2H), 2.25 (s, 4H), 2.20 (br d, J = | |
| | | 17.6 Hz, 3H), 2.09-2.00 (m, 4H), 1.81- | |
| | | 1.45 (m, 6H), 1.25 (d, J = 10.8 Hz, 12H), | |
| | | 1.03 (s, 3H), 0.92 (s, 3H) | |
| 450 | 2-[4-[[4-[5-acetyl-3-[6-cyano-7- | (CDCl₃) δ = 8.68 (s, 2H), 7.57 (d, J = 8.8 | 962.7 |
| | (difluoromethyl)-3,4-dihydro- | Hz, 1H), 7.34 (d, J = 10.8 Hz, 1H), 6.97 | |
| | 2H-quinolin-1-yl]-6,7-dihydro- | (d, J = 2.4 Hz, 1H), 6.91-6.54 (m, 3H), | |
| | 4H-pyrazolo[4,3-c]pyridin-1- | 5.93 (d, J = 8.0 Hz, 1H), 4.86 (br d, J = | |
| | yl]-1-piperidyl]methyl]-3,3- | 12.4 Hz, 1H), 4.50 (br d, J = 13.2 Hz, | |
| | dimethyl-1-piperidyl]-N-[3-(3- | 1H), 4.29 (s, 1H), 4.18-4.07 (m, 2H), | |
| | chloro-4-cyano-phenoxy)- | 4.04 (s, 1H), 4.01-3.85 (m, 2H), 3.83- | |
| | 2,2,4,4-tetramethyl- | 3.66 (m, 3H), 3.26-3.06 (m, 1H), 3.02- | |
| | cyclobutyl]pyrimidine-5- | 2.93 (m, 1H), 2.92-2.80 (m, 4H), 2.79- | |
| | carboxamide | 2.68 (m, 2H), 2.52-2.38 (m, 1H), 2.34- | |
| | | 2.14 (m, 5H), 2.14-2.03 (m, 5H), 2.03- | |
| | | 1.90 (m, 3H), 1.62-1.52 (m, 1H), 1.45- | |
| | | 1.32 (m, 1H), 1.23 (d, J = 14.8 Hz, 12H), | |
| | | 1.05 (s, 3H), 0.78 (s, 3H) | |
| 451 | 4-[4-[[4-[5-acetyl-3-[6-cyano-7- | (CDCl₃) δ = 7.66 (br d, J = 8.8 Hz, 2H), | 956.8 |
| | (difluoromethyl)-3,4-dihydro- | 7.45 (br d, J = 8.4 Hz, 1H), 7.33 (br d, | |
| | 2H-quinolin-1-yl]-6,7-dihydro- | J = 11.6 Hz, 1H), 6.88 (br d, J = 8.8 Hz, | |
| | 4H-pyrazolo[4,3-c]pyridin-1- | 2H), 6.81-6.74 (m, 1H), 6.63-6.38 (m, | |
| | yl]-1-piperidyl]methyl]-3,3- | 2H), 6.11 (br d, J = 8.4 Hz, 1H), 4.32- | |
| | dimethyl-1-piperidyl]-N-[3-(4- | 4.04 (m, 4H), 3.92 (s, 5H), 3.87-3.61 | |
| | cyano-3-methoxy-phenoxy)- | (m, 4H), 3.38 (br d, J = 12.4 Hz, 1H), | |
| | 2,2,4,4-tetramethyl- | 3.19-3.06 (m, 1H), 3.03-2.69 (m, 6H), | |
| | cyclobutyl]benzamide | 2.68-2.55 (m, 1H), 2.48-2.37 (m, 1H), | |
| | | 2.31-2.04 (m, 9H), 1.94 (br s, 4H), 1.46 | |
| | | (br s, 2H), 1.24 (br d, J = 9.2 Hz, 12H), | |
| | | 1.02 (s, 3H), 0.99-0.83 (m, 3H) | |
| 457 | 4-[4-[[4-[5-acetyl-3-(6-cyano-7- | δ = 7.73 (d, J = 8.8 Hz, 2H), 7.64 (d, J = | 920.9 |
| | methyl-3,4-dihydro-2H- | 8.4 Hz, 1H), 7.48 (d, J = 9.2 Hz, 1H), | |
| | quinolin-1-yl)-6,7-dihydro-4H- | 7.32 (s, 1H), 6.94 (d, J = 8.8 Hz, 2H), | |
| | pyrazolo[4,3-c]pyridin-1-yl]-1- | 6.63 (d, J = 2.0 Hz, 1H), 6.53 (d, J = 2.0, | |
| | piperidyl]methyl]-3,5-dimethyl- | 8.8 Hz, 1H), 6.38-6.28 (m, 1H), 4.27 (s, | |
| | 1-piperidyl]-N-[3-(4-cyano-3- | 1H), 4.18-4.09 (m, 2H), 4.09-3.98 (m, | |
| | methoxy-phenoxy)-2,2,4,4- | 2H), 3.90 (s, 3H), 3.80-3.68 (m, 2H), | |
| | tetramethyl- | 3.59 (q, J = 5.6 Hz, 2H), 3.39 (s, 2H), | |
| | cyclobutyl]benzamide | 3.11-2.95 (m, 2H), 2.89-2.68 (m, 6H), | |
| | | 2.34-2.20 (m, 5H), 2.11-1.78 (m, | |
| | | 14H), 1.22 (s, 6H), 1.14 (s, 6H), 1.00 (d, | |
| | | J = 7.2 Hz, 6H) | |
| 458 | 2-[4-[[4-[5-acetyl-3-[6-cyano-7- | δ = 8.73 (s, 2H), 7.90 (d, J = 8.8 Hz, 1H), | 962.6 |
| | (difluoromethyl)-3,4-dihydro- | 7.70 (d, J = 9.2 Hz, 1H), 7.58 (s, 1H), | |
| | 2H-quinolin-1-yl]-6,7-dihydro- | 7.21 (d, J = 2.4 Hz, 1H), 7.17-6.84 (m, | |
| | 4H-pyrazolo[4,3-c]pyridin-1- | 2H), 6.80-6.72 (m, 1H), 4.28 (s, 1H), | |
| | yl]-1-piperidyl]methyl]-3,5- | 4.24-4.13 (m, 2H), 4.10-3.99 (m, 2H), | |
| | dimethyl-1-piperidyl]-N-[3-(3- | 3.98-3.85 (m, 2H), 3.81-3.68 (m, 2H), | |
| | chloro-4-cyano-phenoxy)- | 3.67-3.58 (m, 2H), 3.51-3.40 (m, 2H), | |
| | 2,2,4,4-tetramethyl- | 3.08-2.94 (m, 2H), 2.91-2.71 (m, 4H), | |
| | cyclobutyl]pyrimidine-5- | 2.35-2.24 (m, 2H), 2.10-1.92 (m, 9H), | |
| | carboxamide | 1.91-1.79 (m, 5H), 1.21 (s, 6H), 1.10 (s, | |
| | | 6H), 1.03-0.89 (m, 6H) | |
| 459 | 4-[4-[[4-[5-acetyl-3-[6-cyano-7- | δ = 7.78-7.69 (m, 2H), 7.64 (d, J = 8.4 | 956.7 |
| | (difluoromethyl)-3,4-dihydro- | Hz, 1H), 7.58 (s, 1H), 7.54-7.43 (m, | |
| | 2H-quinolin-1-yl]-6,7-dihydro- | 1H), 7.16-6.85 (m, 3H), 6.80-6.72 (m, | |
| | 4H-pyrazolo[4,3-c]pyridin-1- | 1H), 6.67-6.61 (m, 1H), 6.57-6.49 (m, | |
| | yl]-1-piperidyl]methyl]-3,5- | 1H), 4.27 (s, 1H), 4.22-4.12 (m, 2H), | |
| | dimethyl-1-piperidyl]-N-[3-(4- | 4.10-3.98 (m, 2H), 3.90 (s, 3H), 3.78- | |
| | cyano-3-methoxy-phenoxy)- | 3.69 (m, 2H), 3.67-3.58 (m, 2H), 3.42- | |
| | 2,2,4,4-tetramethyl- | 3.35 (m, 2H), 3.07-2.96 (m, 2H), 2.91- | |
| | cyclobutyl]benzamide | 2.70 (m, 6H), 2.33-2.20 (m, 2H), 2.10- | |
| | | 1.77 (m, 14H), 1.22 (s, 6H), 1.14 (s, 6H), | |
| | | 1.06-0.95 (m, 6H) | |
| 460 | 2-[4-[[4-[5-acetyl-3-[7- | δ = 8.73 (s, 2H), 7.85-7.67 (m, 3H), | 1031.6 |
| | (difluoromethyl)-6-(1- | 7.50 (s, 1H), 7.10 (s, 1H), 6.96-6.61 (m, | |
| | methylpyrazol-4-yl)-3,4- | 3H), 4.26 (s, 1H), 4.20-4.10 (m, 2H), | |
| | dihydro-2H-quinolin-1-yl]-6,7- | 4.08-3.97 (m, 2H), 3.96-3.89 (m, 2H), | |
| | dihydro-4H-pyrazolo[4,3-c] | 3.86 (s, 3H), 3.78-3.65 (m, 2H), 3.63- | |
| | pyridin-1-yl]-1-piperidyl] | 3.55 (m, 2H), 3.52-3.42 (m, 2H), 3.07- | |
| | methyl]-3,5-dimethyl-1- | 2.99 (m, 2H), 2.89-2.81 (m, 3H), 2.77- | |
| | piperidyl]-N-[3-(3-chloro-4- | 2.70 (m, 1H), 2.36-2.29 (m, 5H), 2.11- | |
| | cyano-2-methyl-phenoxy)- | 1.93 (m, 9H), 1.93-1.81 (m, 5H), 1.22 | |
| | 2,2,4,4-tetramethyl-cyclobutyl] | (s, 6H), 1.12 (s, 6H), 0.94 (br d, J = 6.8 | |
| | pyrimidine-5-carboxamide | Hz, 6H) | |
| 462 | 2-[4-[[4-[5-acetyl-3-[7- | δ = 8.73 (s, 2H), 7.75 (s, 1H), 7.69 (d, J = | 997.7 |
| | (difluoromethyl)-6-(1- | 8.8 Hz, 2H), 7.50 (s, 1H), 7.10 (s, 1H), | |
| | methylpyrazol-4-yl)-3,4- | 6.98-6.62 (m, 4H), 4.22-4.09 (m, 3H), | |
| | dihydro-2H-quinolin-1-yl]-6,7- | 4.08-3.83 (m, 7H), 3.77-3.66 (m, 2H), | |
| | dihydro-4H-pyrazolo[4,3-c] | 3.62-3.55 (m, 2H), 3.51-3.42 (m, 2H), | |
| | pyridin-1-yl]-1-piperidyl] | 3.02 (br d, J = 5.6 Hz, 2H), 2.85 (br d, | |
| | methyl]-3,5-dimethyl-1- | J = 6.0 Hz, 3H), 2.77-2.68 (m, 1H), 2.44 | |
| | piperidyl]-N-[3-(4-cyano-3- | (s, 3H), 2.31 (br s, 2H), 2.07 (s, 2H), | |
| | methyl-phenoxy)-2,2,4,4- | 2.05-1.80 (m, 11H), 1.47-1.16 (m, 7H), | |
| | tetramethyl-cyclobutyl] | 1.10 (s, 6H), 1.04-0.89 (m, 6H) | |
| | pyrimidine-5-carboxamide | | |
| 463 | 2-[4-[[4-[5-acetyl-3-[7- | (CDCl₃) δ = 8.75-8.64 (m, 2H), 7.75 (d, | 1051.7 |
| | (difluoromethyl)-6-(1- | J = 8.8 Hz, 1H), 7.53 (d, J = 5.6 Hz, 1H), | |
| | methylpyrazol-4-yl)-3,4- | 7.40 (d, J = 6.4 Hz, 1H), 7.24 (d, J = 2.4 | |
| | dihydro-2H-quinolin-1-yl]-6,7- | Hz, 1H), 7.08-6.97 (m, 2H), 6.92-6.83 | |
| | dihydro-4H-pyrazolo[4,3- | (m, 1H), 6.68-6.37 (m, 1H), 5.94 (d, J = | |
| | c]pyridin-1-yl]-1- | 8.4 Hz, 1H), 4.68-4.64 (m, 1H), 4.32- | |
| | piperidyl]methyl]-3,5-dimethyl- | 4.21 (m, 1H), 4.17-4.09 (m, 3H), 4.03- | |
| | 1-piperidyl]-N-[3-[4-cyano-3- | 3.95 (m, 4H), 3.91 (d, J = 5.6 Hz, 2H), | |
| | (trifluoromethyl)phenoxy]- | 3.80-3.66 (m, 3H), 3.44-3.31 (m, 1H), | |
| | 2,2,4,4-tetramethyl- | 3.18-3.01 (m, 2H), 2.94-2.60 (m, 5H), | |
| | cyclobutyl]pyrimidine-5- | 2.38 (s, 2H), 2.31-2.10 (m, 6H), 2.10- | |
| | carboxamide | 2.03 (m, 4H), 1.94 (s, 4H), 1.29-1.21 | |
| | | (m, 12H), 1.09-1.00 (m, 6H) | |
| 465 | 2-[4-[[4-[5-acetyl-3-[7-methyl- | (CDCl₃) δ = 8.62 (s, 2H), 7.51-7.44 (m, | 975.7 |
| | 6-(1-methylpyrazol-4-yl)-3,4- | 1H), 7.38-7.25 (m, 2H), 6.98-6.82 (m, | |
| | dihydro-2H-quinolin-1-yl]-6,7- | 1H), 6.45 (d, J = 8.8 Hz, 1H), 6.31 (s, | |
| | dihydro-4H-pyrazolo[4,3- | 1H), 5.87 (d, J = 8.4 Hz, 1H), 4.86-4.71 | |
| | c]pyridin-1-yl]-1- | (m, 1H), 4.42 (br d, J = 12.8 Hz, 1H), | |
| | piperidyl]methyl]-3,3-dimethyl- | 4.25-4.03 (m, 3H), 3.99 (s, 1H), 3.89- | |
| | 1-piperidyl]-N-[3-(4-cyano-2,3- | 3.86 (m, 3H), 3.67 (brt, J = 5.6 Hz, 3H), | |
| | dimethyl-phenoxy)-2,2,4,4- | 3.64-3.53 (m, 2H), 3.11-2.97 (m, 1H), | |
| | tetramethyl- | 2.91-2.79 (m, 2H), 2.79-2.70 (m, 3H), | |
| | cyclobutyl]pyrimidine-5- | 2.70-2.60 (m, 2H), 2.42 (s, 3H), 2.37- | |
| | carboxamide | 2.28 (m, 1H), 2.26-2.08 (m, 10H), 2.07- | |
| | | 1.95 (m, 5H), 1.94-1.81 (m, 4H), 1.51- | |
| | | 1.44 (m, 1H), 1.36-1.27 (m, 1H), 1.17 | |
| | | (d, J = 10.4 Hz, 12H), 0.98 (s, 3H), 0.72 | |
| | | (s, 3H) | |
| 466 | 2-[4-[[4-[5-acetyl-3-[7-methyl- | δ = 8.61 (s, 2H), 7.54-7.40 (m, 2H), | 961.8 |
| | 6-(1-methylpyrazol-4-yl)-3,4- | 7.36-7.26 (m, 1H), 6.96-6.85 (m, 1H), | |
| | dihydro-2H-quinolin-1-yl]-6,7- | 6.71 (d, J = 2.0 Hz, 1H), 6.65-6.58 (m, | |
| | dihydro-4H-pyrazolo[4,3- | 1H), 6.31 (s, 1H), 5.86 (d, J = 8.0 Hz, | |
| | c]pyridin-1-yl]-1- | 1H), 4.79 (br d, J = 13.6 Hz, 1H), 4.43 | |
| | piperidyl]methyl]-3,3-dimethyl- | (br d, J = 13.2 Hz, 1H), 4.25-4.00 (m, | |
| | 1-piperidyl]-N-[3-(4-cyano-3- | 3H), 3.97 (s, 1H), 3.91-3.54 (m, 8H), | |
| | methyl-phenoxy)-2,2,4,4- | 3.06 (br d, J = 10.8 Hz, 1H), 2.97-2.60 | |
| | tetramethyl- | (m, 7H), 2.44 (s, 3H), 2.41-2.32 (m, | |
| | cyclobutyl]pyrimidine-5- | 1H), 2.29-2.01 (m, 9H), 2.00-1.89 (m, | |
| | carboxamide | 7H), 1.51 (br d, J = 10.8 Hz, 1H), 1.37- | |
| | | 1.27 (m, 1H), 1.16 (d, J = 12.0 Hz, 12H), | |
| | | 0.98 (s, 3H), 0.72 (s, 3H) | |

### Example 66: Synthesis of 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3, 5-dimethyl-1-piperidyl]-N-[3-(4-cyano-2,3-dimethyl-phenoxy)-2,2,4,4-tetramethyl-cyclobutyl]pyrimidine-5-carboxamide (Compound 461)

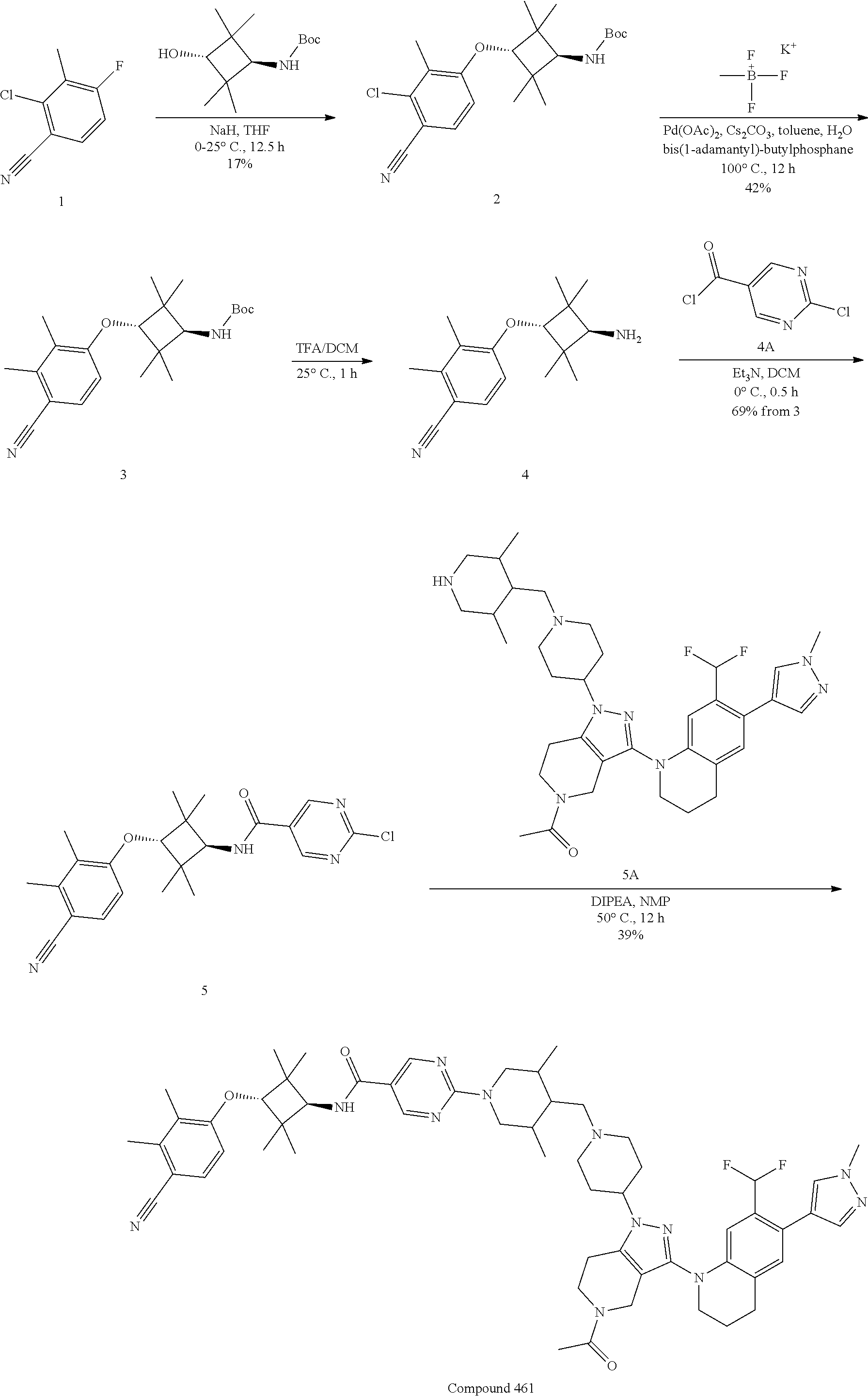

Synthesis of 4A was reported in WO2017/181177, 2017, A1

To a solution of 2-chloro-4-fluoro-3-methyl-benzonitrile (500 mg, 2.95 mmol, 1.0 eq), tert-butyl N-(3-hydroxy-2,2,4,4-tetramethyl-cyclobutyl)carbamate (718 mg, 2.95 mmol, 1.0 eq) in THF (10 mL) was added NaH (259 mg, 60% w/w, 6.49 mmol, 2.2 eq). The mixture was stirred at 0° C. for 0.5 h under N₂. And then 2-chloro-4-fluoro-3-methyl-benzonitrile (500 mg, 2.95 mmol, 1.0 eq) was added at 0° C. The mixture was stirred at 25° C. for 12 h under N₂. The reaction mixture was quenched with saturated aqueous NH₄Cl (20 mL) solution at 0° C. under N₂. And the resulting mixture was extracted with ethyl acetate (50 mL×3). The combined organic layers were dried over anhydrous Na₂SO₄, filtered and concentrated in vacuum. The residue was purified by column chromatography (SiO₂, petroleum ether/ethyl acetate=50/1 to 5/1). The desired compound tert-butyl N-[3-(3-chloro-4-cyano-2-methyl-phenoxy)-2,2,4,4-tetramethyl-cyclobutyl]carbamate (200 mg, 509 μmol, 17% yield) was obtained as yellow solid.

LC-MS: MS (ESI⁺): t_{R}=1.133 min, m/z=337.1 [M−55]

To a solution of tert-butyl N-[3-(3-chloro-4-cyano-2-methyl-phenoxy)-2,2,4,4-tetramethyl-cyclobutyl]carbamate (200 mg, 509 mol, 1.0 eq), potassium; trifluoro(methyl)boranuide (200 mg, 1.64 mmol, 3.2 eq) in toluene (6 mL), H₂O (2 mL) was added Pd(OAc)₂ (20 mg, 89 μmol, 0.2 eq), bis(1-adamantyl)-butyl-phosphane (40 mg, 112 μmol, 0.2 eq), Cs₂CO₃ (350 mg, 1.07 mmol, 2.1 eq). The mixture was stirred at 100° C. for 12 h. The reaction was diluted with water (60 mL) and the resulting mixture was extracted with ethyl acetate (30 mL×2). The combined organic layers were dried over Na₂SO₄ and concentrated to give the residue. The residue was purified by prep-TLC (SiO₂, petroleum ether/ethyl acetate=3/1). Compound tert-butyl N-[3-(4-cyano-2,3-dimethyl-phenoxy)-2,2,4,4-tetramethyl-cyclobutyl]carbamate (80 mg, 215 mol, 42% yield) was obtained as a yellow oil.

LC-MS: MS (ESI⁺): t_{R}=0.617 min, m/z=373.2 [M+H⁺]

To a solution of tert-butyl N-[3-(4-cyano-2,3-dimethyl-phenoxy)-2,2,4,4-tetramethyl-cyclobutyl]carbamate (80 mg, 215 mol, 1.0 eq) in DCM (2 mL) was added TFA (1.54 g, 13.5 mmol, 62.7 eq). The mixture was stirred at 25° C. for 0.5 h. The reaction mixture was concentrated under reduced pressure to give a residue. Compound 4-(3-amino-2,2,4,4-tetramethyl-cyclobutoxy)-2,3-dimethyl-benzonitrile (58 mg) was obtained as colourless oil and directly used in the next step without further purification.

LC-MS: MS (ESI⁺): t_{R}=0.617 min, m/z=273.2 [M+H⁺]

To a solution of 4-(3-amino-2,2,4,4-tetramethyl-cyclobutoxy)-2,3-dimethyl-benzonitrile (58 mg) in DCM (1 mL) was added Et₃N (43 mg, 426 μmol, 2.0 eq). Followed by 2-chloropyrimidine-5-carbonyl chloride (45 mg, 256 μmol, 1.2 eq) in DCM (1 mL) at 0° C. The mixture was stirred at 0° C. for 0.5 h. The reaction mixture was concentrated under reduced pressure to give a residue. The residue was purified by prep-TLC (SiO₂, petroleum ether/ethyl acetate=1/1). The desired compound 2-chloro-N-[3-(4-cyano-2,3-dimethyl-phenoxy)-2,2,4,4-tetramethyl-cyclobutyl]pyrimidine-5-carboxamide (61 mg, 148 μmol, 69% yield over two steps) as obtained as off-white solid.

LC-MS: MS (ESI⁺): t_{R}=0.664 min, m/z=413.1 [M+H⁺]

To a solution of 1-[3-[7-(difluoromethyl)-6-(1-methylpyrazol-4-yl)-3,4-dihydro-2H-quinolin-1-yl]-1-[1-[(3,5-dimethyl-4-piperidyl)methyl]-4-piperidyl]-6,7-dihydro-4H-pyrazolo[4,3-c]pyridin-5-yl]ethanone (85 mg, 134 μmol, 1.0 eq), DIPEA (87 mg, 672 μmol, 5.0 eq) in NMP (2 mL) was added 2-chloro-N-[3-(4-cyano-2,3-dimethyl-phenoxy)-2,2,4,4-tetramethyl-cyclobutyl]pyrimidine-5-carboxamide (61 mg, 148 μmol, 1.1 eq). The mixture was stirred at 50° C. for 12 h. The reaction mixture was concentrated under reduced pressure to give a residue. The residue was purified by prep-HPLC (FA condition; column: Phenomenex Luna C18 150×25 mm×10 um; mobile phase: [H₂O (0.225% FA)-ACN]; gradient: 32%-62% B over 10.0 min). The desired compound 2-[4-[[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,5-dimethyl-1-piperidyl]-N-[3-(4-cyano-2,3-dimethyl-phenoxy)-2,2,4,4-tetramethyl-cyclobutyl]pyrimidine-5-carboxamide (52.63 mg, 52.04 μmol, 39% yield) was obtained as off-white solid.

¹H NMR (400 MHz, CHLOROFORM-d) δ=8.78-8.61 (m, 2H), 7.53 (d, J=5.6 Hz, 1H), 7.45-7.36 (m, 2H), 7.07-6.97 (m, 1H), 6.92-6.83 (m, 1H), 6.70-6.35 (m, 2H), 5.95 (d, J=8.0 Hz, 1H), 4.68-4.26 (m, 1H), 4.18-4.10 (m, 2H), 4.06 (s, 1H), 4.04-3.95 (m, 4H), 3.95-3.67 (m, 5H), 3.57-2.93 (m, 4H), 2.92-2.52 (m, 5H), 2.49 (s, 3H), 2.38 (s, 2H), 2.30-2.21 (m, 5H), 2.19-2.02 (m, 7H), 1.99-1.80 (m, 3H), 1.58-1.44 (m, 1H), 1.24 (d, J=9.2 Hz, 12H), 1.09-0.99 (m, 6H)

LC-MS: MS (ESI⁺): t_{R}=3.186 min, m/z=1011.7 [M+H⁺]

The compounds below were prepared in a similar manner as described in Example 66.

| | | | LC-MS |
|---|---|---|---|
| Compound | Name | ¹H NMR (DMSO-d6) | [M + H⁺] |
| 462 | 2-[4-[[4-[5-acetyl-3-[7- | δ = 8.73 (s, 2H), 7.75 (s, 1H), 7.69 (d, J = | 997.7 |
| | (difluoromethyl)-6-(1- | 8.8 Hz, 2H), 7.50 (s, 1H), 7.10 (s, 1H), | |
| | methylpyrazol-4-yl)-3,4- | 6.98-6.62 (m, 4H), 4.22-4.09 (m, 3H), | |
| | dihydro-2H-quinolin-1-yl]-6,7- | 4.08-3.83 (m, 7H), 3.77-3.66 (m, 2H), | |
| | dihydro-4H-pyrazolo[4,3-c] | 3.62-3.55 (m, 2H), 3.51-3.42 (m, 2H), | |
| | pyridin-1-yl]-1-piperidyl] | 3.02 (br d, J = 5.6 Hz, 2H), 2.85 (br d, | |
| | methyl]-3,5-dimethyl-1- | J = 6.0 Hz, 3H), 2.77-2.68 (m, 1H), 2.44 | |
| | piperidyl]-N-[3-(4-cyano-3- | (s, 3H), 2.31 (br s, 2H), 2.07 (s, 2H), | |
| | methyl-phenoxy)-2,2,4,4- | 2.05-1.80 (m, 11H), 1.47-1.16 (m, 7H), | |
| | tetramethyl-cyclobutyl] | 1.10 (s, 6H), 1.04-0.89 (m, 6H) | |
| | pyrimidine-5-carboxamide | | |

### Example 67: Synthesis of 2-[4-[[4-[5-acetyl-3-[7-methyl-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,3-dimethyl-1-piperidyl]-N-[3-(3-chloro-4-cyano-2-methyl-phenoxy)-2,2,4,4-tetramethyl-cyclobutyl]pyrimidine-5-carboxamide (Compound 464)

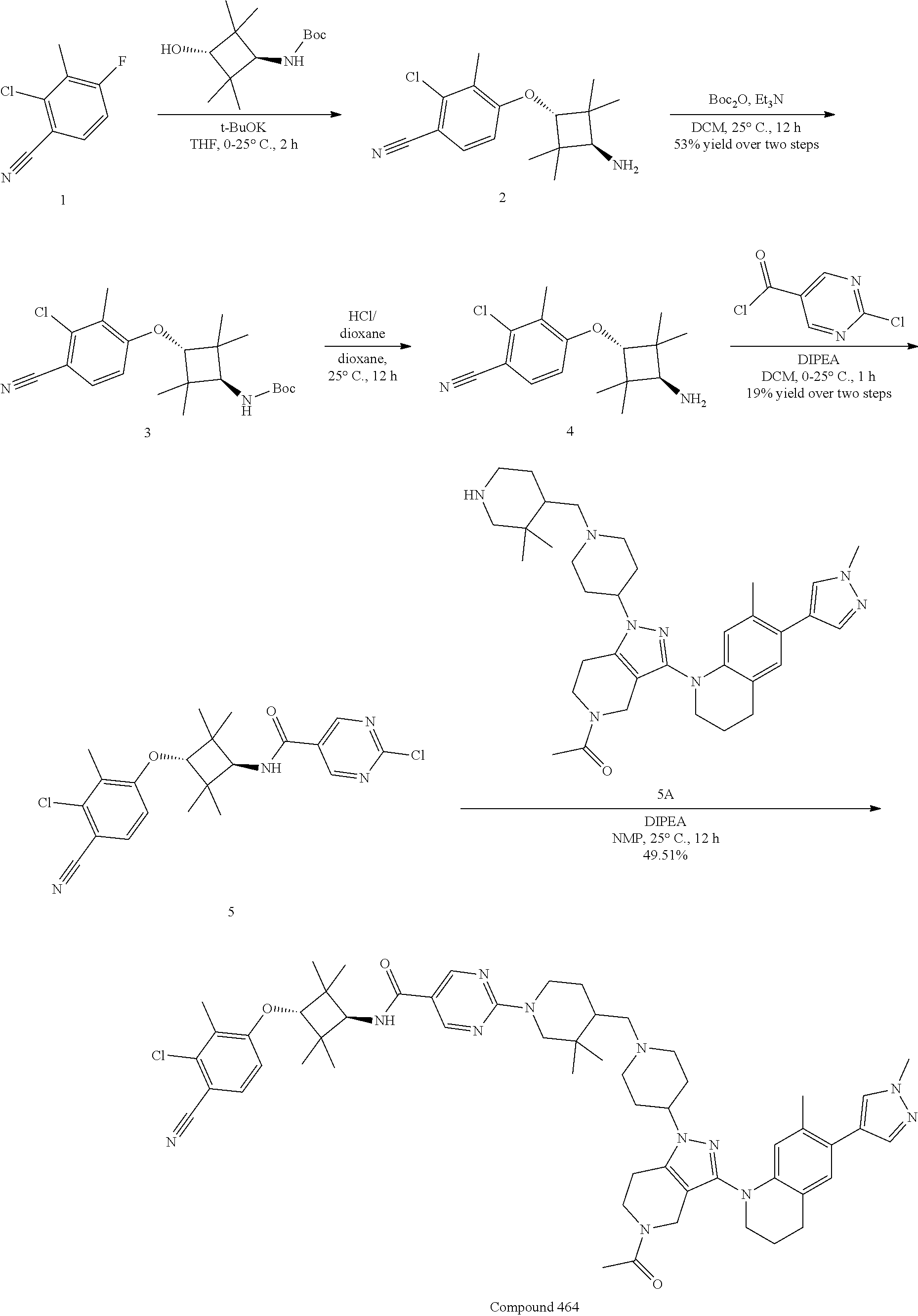

To a solution of 2-chloro-4-fluoro-3-methyl-benzonitrile (3.48 g, 21 mmol, 1.0 eq) and tert-butyl N-(3-hydroxy-2,2,4,4-tetramethyl-cyclobutyl)carbamate (5.00 g, 21 mmol, 1.0 eq) in THF (15.0 mL) was added t-BuOK (1 M, 61.60 mL, 3.0 eq) at 0° C. The mixture was stirred at 25° C. for 2 h. The mixture was poured into water (100 mL) at 0° C. The aqueous phase was extracted with EtoAc (100 mL×3). The combined organic phase was washed with brine (100 mL×2), dried over anhydrous Na₂SO₄, filtered, and concentrated to give a residue. Compound 4-(3-amino-2,2,4,4-tetramethyl-cyclobutoxy)-2-chloro-3-methyl-benzonitrile (6.00 g) was obtained as a yellow solid was used into the next step without further purification.

LC-MS: MS (ESI⁺): t_{R}=0.738 min, m/z=293.0 [M+H⁺]

To a solution of 4-(3-amino-2,2,4,4-tetramethyl-cyclobutoxy)-2-chloro-3-methyl-benzonitrile (6.00 g) in DCM (60 mL) was added ET₃N (4.20 g, 41 mmol, 5.70 mL, 2.0 eq) and Boc₂O (4.50 g, 20 mmol, 4.70 mL, 1.0 eq). The mixture was stirred at 25° C. for 12 h. The reaction mixture was filtered and concentrated to give a residue. The residue was purified by column chromatography (SiO₂, Petroleum ether: Ethyl acetate=20:1 to 10:1). The crude product was triturated with PE:EA (3:1 30 ml) at 25° C. for 30 min. Compound tert-butyl N-[3-(3-chloro-4-cyano-2-methyl-phenoxy)-2,2,4,4-tetramethyl-cyclobutyl]carbamate (4.30 g, 11 mmol, 53% yield over two steps) was obtained as a yellow solid.

¹H NMR (400 MHz, CDCl₃): δ=7.47 (d, J=8.4 Hz, 1H), 6.58 (d, J=8.8 Hz, 1H), 4.70 (br d, J=7.6 Hz, 1H), 4.01 (s, 1H), 3.73 (br d, J=8.4 Hz, 1H), 2.37 (s, 3H), 1.48 (s, 9H), 1.31-1.06 (m, 12H).

LC-MS: MS (ESI⁺): t_{R}=0.735 min, m/z=337.1 [M−55]

To a solution of tert-butyl N-[3-(3-chloro-4-cyano-2-methyl-phenoxy)-2,2,4,4-tetramethyl-cyclobutyl]carbamate (1.00 g, 3 mmol, 1.0 eq) in dioxane (2 mL) was added HCl/dioxane (10 mL). The mixture was stirred at 25° C. for 12 h. The mixture was filtered and concentrated to give a residue. Compound 4-(3-amino-2,2,4,4-tetramethyl-cyclobutoxy)-2-chloro-3-methyl-benzonitrile (745 mg) was obtained as a white solid was used into the next step without further purification.

LC-MS: MS (ESI⁺): t_{R}=0.509 min, m/z=293.1 [M+H⁺]

To a solution of 2-chloropyrimidine-5-carbonyl chloride (495 mg, 3 mmol, 1.1 eq) in DCM (10 mL) was added DIPEA (987 mg, 8 mmol, 1.30 mL, 3.0 eq) at 0° C., then 4-(3-amino-2,2,4,4-tetramethyl-cyclobutoxy)-2-chloro-3-methyl-benzonitrile (745 mg) was added. The mixture was stirred at 25° C. for 1 h. The reaction mixture was diluted with water (10 mL) and the mixture was extracted with dichloromethane (10 mL*3). The combined organic phase was washed with brine (20 mL), dried over anhydrous Na₂SO₄, filtered, and concentrated to give a residue. The was triturated with PE:EA=0:1 (20 mL) at 25° C. for 0.5 h. Compound 2-chloro-N-[3-(3-chloro-4-cyano-2-methyl-phenoxy)-2,2,4,4-tetramethyl-cyclobutyl]pyrimidine-5-carboxamide (230 mg, 477 mol, 19% yield over two steps) was obtained as a white solid.

LC-MS: MS (ESI⁺): t_{R}=0.684 min, m/z=433.1 [M+H⁺]

¹H NMR (400 MHz, CDCl₃): δ=8.92 (s, 2H), 7.40 (d, J=8.8 Hz, 1H), 6.51 (d, J=8.8 Hz, 1H), 6.11 (br d, J=7.6 Hz, 1H), 4.12 (d, J=8.4 Hz, 1H), 4.04 (s, 1H), 2.32 (s, 3H), 1.21 (d, J=17.6 Hz, 12H).

To a solution of 2-chloro-N-[3-(3-chloro-4-cyano-2-methyl-phenoxy)-2,2,4,4-tetramethyl-cyclobutyl]pyrimidine-5-carboxamide (60 mg, 125 mol, 1.0 eq) and 1-[1-[1-[(3,3-dimethyl-4-piperidyl)methyl]-4-piperidyl]-3-[7-methyl-6-(1-methylpyrazol-4-yl)-3,4-dihydro-2H-quinolin-1-yl]-6,7-dihydro-4H-pyrazolo[4,3-c]pyridin-5-yl]ethanone (75 mg, 125 μmol, 1.0 eq) in NMP (0.5 mL) was added DIPEA (49 mg, 376 μmol, 65 μL, 3.0 eq). The mixture was stirred at 25° C. for 12 h. The mixture was filtered and concentrated under reduced pressure to give a residue. The residue was purified by prep-HPLC (column: Phenomenex Luna C18 150*25 mm*10 um; mobile phase: [H₂O (0.225% FA)-ACN]; gradient: 34%-64% B over 10.0 min). Compound 2-[4-[[4-[5-acetyl-3-[7-methyl-6-(1-methylpyrazol-4-yl)-3,4-dihydro-2H-quinolin-1-yl]-6,7-dihydro-4H-pyrazolo[4,3-c]pyridin-1-yl]-1-piperidyl]methyl]-3,3-dimethyl-1-piperidyl]-N-[3-(3-chloro-4-cyano-2-methyl-phenoxy)-2,2,4,4-tetramethyl-cyclobutyl]pyrimidine-5-carboxamide (62.78 mg, 62.00 μmol, 49.51% yield) was obtained as a white solid.

¹H NMR (400 MHz, CDCl₃) δ=8.61 (s, 2H), 7.53-7.26 (m, 3H), 6.97-6.84 (m, 1H), 6.51 (d, J=8.8 Hz, 1H), 6.37-6.28 (m, 1H), 5.87 (d, J=8.4 Hz, 1H), 4.79 (br d, J=12.8 Hz, 1H), 4.42 (br d, J=13.2 Hz, 1H), 4.26-3.97 (m, 4H), 3.93-3.81 (m, 5H), 3.74-3.52 (m, 3H), 3.04 (br d, J=10.4 Hz, 1H), 2.94-2.60 (m, 7H), 2.40-2.29 (m, 4H), 2.27-2.12 (m, 6H), 2.10 (s, 1H), 2.08-1.92 (m, 6H), 1.92-1.80 (m, 3H), 1.56-1.42 (m, 1H), 1.38-1.26 (m, 1H), 1.18 (d, J=11.6 Hz, 12H), 0.98 (s, 3H), 0.72 (s, 3H).

LC-MS: MS (ESI⁺): t_{R}=2.575 min, m/z=995.8 [M+H⁺]

### Example A-1: Oral Solution

To prepare a pharmaceutical composition for oral delivery, a sufficient amount of a compound of Formula (I) or (II), or a pharmaceutically acceptable salt thereof, is added to water (with optional solubilizer(s), optional buffer(s) and taste masking excipients) to provide a 20 mg/mL solution.

### Example A-2: Oral Tablet

A tablet is prepared by mixing 20-50% by weight of a compound of Formula (I) or (II), or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-3: Oral Capsule

To prepare a pharmaceutical composition for oral delivery, 10-500 mg of a compound of Formula (I) or (II), or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound of Formula (I) or (II), or a pharmaceutically acceptable salt thereof, is placed into Size 4 capsule, or size 1 capsule (hypromellose or hard gelatin) and the capsule is closed.

### Example B-1: Ternary Complex Formation Assay

### Preparation of Mouse Anti-AR Antibody Coated 96 Well Plates

Black MaxiSorp plates: coat with 50 μL of mouse 1/50 diluted AR antibody (Monoclonal Antibody (AR 441 from Thermo Fisher); diluted in phosphate buffered saline (PBS). Spin the liquid to the bottom of the wells, if necessary. Incubate overnight at 4° C. Next day, wash 3 times with 200 μL of TBST. Block with 200 μL of blocking buffer (5% BSA in TBST) at room temperature for 2 hours (BSA: A4503 or A2153 from Sigma). Wash plates 3 times with TBST. Plates are ready for lysates Preparation of dimerizer treated lysates

Culture cells in 96 wells at 50K VCaP cells/well for at least 3 days. Plate in 5% Omega charcoal stripped FBS in total volume of 200 uL. Treat with bifunctional for 1.5 hours by adding 1 μL of 200× stock in DMSO. Aspirate media/compound. Add and pipet-to-mix 70 μL of 1× AlphaLisa Lysis Buffer to each well (Perkin Elmer, AlphaLISA SureFire Ultra, diluted in water). Pipet up and down when adding lysis buffer to disrupt cells. Let lysis proceed for 30 min at 4° C.

### Sandwich ELISA

Transfer 50 μL of lysate to MaxiSorp plates coated with antibody and subsequently blocked with BSA. Incubate for 1 hour at room temperature. Flick out the lysates. Wash 3 times with TBST. Add 75 μL of rabbit CBP or p300 antibody to each well (diluted 1:5000 in blocking buffer (5% BSA in TBST). CBP antibody: (D6C5) Rabbit mAb #7389; p300 antibody: (D8Z4E) Rabbit mAb #86377. Incubate overnight at 4° C. Wash 3 times with 300 uL TBST, each time on shaker for 5-10 min. Prepare goat anti-rabbit antibody conjugated to HRP (1/100K dilution in blocking buffer) Invitrogen Catalog number: 32260. Add 75 μL of HRP-conjugated goat anti-rabbit antibody. Incubate at room temperature for 1 hour. Flick out the HRP-antibody mixture. Wash 7-8 times with TBST, each time on shaker for 10 min. Later washes try to overflow the wells so that no residual HRP remains bound to the sides of the well. Prepare and add 75 uL/well of QuantaRed Enhance Chemifluorescent HRP Substrate solution. Immediately read fluorescence on BMG ClarioStar (ex: 570/em 585), and repeat the read in 5 minutes and 15 minutes.

Results of the ternary complex formation assay are shown in Table 2.

TABLE 2

| | Average |
|---|---|
| | Ternary |
| | Complex* |
| Compound | (nM) |
| 1 | A |
| 3 | D |
| 4 | D |
| 5 | D |
| 6 | D |
| 8 | D |
| 9 | A |
| 10 | D |
| 11 | C |
| 12 | B |
| 13 | B |
| 15 | C |
| 18 | A |
| 19 | A |
| 20 | A |
| 21 | B |
| 22 | A |
| 24 | A |
| 25 | B |
| 26 | A |
| 27 | A |
| 28 | A |
| 41 | A |
| 42 | A |
| 43 | D |
| 44 | D |
| 45 | D |
| 46 | A |
| 48 | D |
| 49 | D |
| 50 | A |
| 51 | A |
| 53 | A |
| 54 | D |
| 55 | D |
| 56 | A |
| 57 | D |
| 58 | D |
| 59 | D |
| 60 | D |
| 64 | A |
| 65 | A |
| 66 | A |
| 69 | A |
| 70 | A |
| 71 | A |
| 72 | A |
| 74 | A |
| 75 | D |
| 76 | A |
| 77 | A |
| 78 | A |
| 79 | A |
| 82 | A |
| 83 | A |
| 84 | A |
| 85 | A |
| 86 | D |
| 87 | A |
| 89 | A |
| 90 | A |
| 91 | D |
| 92 | D |
| 93 | D |
| 94 | C |
| 95 | B |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 111 | A |
| 112 | A |
| 113 | A |
| 114 | A |
| 115 | A |
| 116 | D |
| 117 | B |
| 118 | D |
| 119 | A |
| 120 | A |
| 122 | A |
| 127 | A |
| 128 | D |
| 129 | A |
| 131 | A |
| 135 | A |
| 136 | A |
| 137 | A |
| 138 | A |
| 139 | D |
| 140 | A |
| 141 | A |
| 142 | A |
| 143 | A |
| 144 | A |
| 150 | B |
| 152 | D |
| 155 | A |
| 157 | A |
| 158 | B |
| 160 | D |
| 161 | C |
| 162 | A |
| 163 | A |
| 167 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 185 | A |
| 186 | A |
| 188 | A |
| 193 | D |
| 209 | C |
| 212 | C |
| 213 | D |
| 214 | A |
| 215 | A |
| 216 | A |
| 217 | A |
| 218 | D |
| 219 | B |
| 220 | A |
| 221 | B |
| 222 | A |
| 223 | A |
| 224 | A |
| 225 | A |
| 226 | A |
| 227 | A |
| 228 | B |
| 229 | B |
| 230 | C |
| 231 | C |
| 232 | C |
| 233 | B |
| 234 | B |
| 235 | A |
| 236 | D |
| 237 | A |
| 238 | C |
| 239 | A |
| 240 | B |
| 241 | B |
| 242 | D |
| 243 | D |
| 244 | B |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | B |
| 259 | B |
| 263 | A |
| 264 | A |
| 265 | A |
| 267 | B |
| 268 | A |
| 271 | A |
| 273 | A |
| 274 | A |
| 275 | A |
| 277 | A |
| 278 | A |
| 280 | A |
| 282 | A |
| 284 | A |
| 286 | D |
| 287 | B |
| 288 | A |
| 289 | A |
| 292 | A |
| 293 | A |
| 294 | A |
| 295 | A |
| 296 | A |
| 299 | A |
| 302 | A |
| 304 | A |
| 305 | A |
| 307 | B |
| 308 | A |
| 309 | A |
| 310 | A |
| 313 | A |
| 314 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 329 | A |
| 331 | A |
| 332 | A |
| 333 | D |
| 334 | A |
| 335 | A |
| 336 | A |
| 337 | A |
| 338 | A |
| 339 | A |
| 340 | A |
| 341 | A |
| 342 | A |
| 343 | A |
| 344 | A |
| 345 | A |
| 346 | A |
| 347 | A |
| 348 | A |
| 349 | A |
| 350 | A |
| 356 | A |
| 357 | A |
| 360 | A |
| 362 | A |
| 363 | A |
| 364 | A |
| 367 | A |
| 369 | A |
| 370 | A |
| 371 | A |
| 372 | A |
| 373 | A |
| 374 | A |
| 375 | A |
| 376 | A |
| 377 | B |
| 378 | B |
| 379 | B |
| 380 | B |
| 381 | A |
| 382 | A |
| 383 | A |
| 384 | A |
| 385 | A |
| 386 | A |
| 387 | A |
| 388 | A |
| 389 | A |
| 390 | A |
| 391 | A |
| 392 | A |
| 395 | A |
| 396 | A |
| 397 | B |
| 398 | B |
| 399 | B |
| 400 | A |
| 401 | A |
| 402 | A |
| 403 | D |
| 404 | A |
| 405 | C |
| 406 | B |
| 407 | C |
| 408 | D |
| 410 | D |
| 412 | D |
| 413 | A |
| 414 | A |
| 415 | B |
| 416 | B |
| 422 | A |
| 424 | A |
| 425 | A |
| 426 | A |
| 428 | A |
| 431 | A |
| 432 | A |
| 433 | A |
| 434 | A |
| 435 | A |
| 437 | A |
| 440 | A |
| 441 | A |
| 442 | A |
| 443 | A |
| 444 | A |
| 446 | A |
| 447 | A |
| 448 | A |
| 449 | A |
| 450 | A |
| 451 | A |
| 452 | A |
| 453 | A |
| 455 | A |
| 456 | A |
| 457 | A |
| 458 | A |
| 459 | A |
| 460 | A |
| 461 | A |
| 462 | A |
| 463 | A |
| 464 | A |
| 465 | A |
| 466 | A |
| 467 | A |
| 468 | A |
| 469 | A |
| 470 | B |
| 471 | A |
| 472 | A |
| 473 | A |

*A is <1 nM; B is >1 nM and ≤10 nM; C is >10 nM and ≤100 nM; D is active and >100 nM

### Example B-2: NanoBRET Cellular Assay

NanoBRET™ Histone H3.3/CBP-BD Interaction Assay Reagents and FuGENE® HD Transfection Reagents were purchased from Promega. Reference compound SGC-CBP-30 was purchased from MedChem Express. HEK293 cell line was purchased from American Type Culture Collection (Manassas, VA). HEK293 cells were cultured in EMEM media supplemented with 10% FBS, 100 μg/ml of penicillin, and 100 μg/ml of streptomycin. Cultures were maintained at 37° C. in a humidified atmosphere of 5% CO₂ and 95% air.

HEK293 cells were transfected with 2 μg of Histone H3.3-HaloTag Fusion Vector DNA plus 0.05 ug of CBP-BD-NanoLuc fusion vector. The transfected cells were treated with NanoBRET™ 618 Ligand and test compounds (starting at 1 uM, 10-dose with 3-fold dilution) or with reference compound SGC-CBP-30 (starting at 10 uM, 10-dose with 3-fold dilution) for 22 hours. Cellular Histone H3.3/CBP-BD Interaction was measured by NanoBRET assay. Curve fits were performed only when % NanoBret signal at the highest concentration of compounds was less than 55%.

Results of the NanoBRET assay are shown in Table 3.

TABLE 3

| Compound | IC₅₀ value (M) |
|---|---|
| GNE-781 | 1.23E−10 |
| Compound 109 | >1.0E−06 |
| SGC-CBP-30 | 6.09E−07 |

Transfection for NanoBRET™ Histone H3.3/CBP-BD Interaction Assay
1. Culture HEK293 cells appropriately prior to assay.
2. Remove medium from cell flask by aspiration, trypsinize and allow cells to dissociate from the flask bottom.
3. Neutralize trypsin using cell culture medium, count cells to estimate density and resuspend to a final density of 4×10⁵ cells/ml in cell culture medium.
4. Plate 2 ml of cells into each well of a six-well plate.
5. Allow the cells to attach and recover overnight at 37° C., 5% CO₂.
6. Prepare a transfection mixture consisting of the following DNA amounts: 2 μg of Histone H3.3-HaloTag 0.05 μg of NanoLuc-CBP-BD
7. Add 100 μl of Opti-MEM® I Reduced Serum Medium, no phenol red, to the transfection mixture, and mix well.
8. Add 8 μl of FuGENE® HD Transfection Reagent and incubate at room temperature for 10 minutes.
9. Add transfection mixture to wells with attached cells, and express proteins for 24 hours at 37° C., 5% CO₂.
   Replating Transfected HEK293 Cells into Multiwell Plates and Adding HaloTag® NanoBRET™ 618 Ligand
1. For each well in a six-well plate, remove medium from cells, and wash with 1 ml of PBS. Discard.
2. Add 0.25% trypsin-EDTA and incubate at room temperature until cells lift from well bottom.
3. Add cell culture medium to neutralize trypsin, mix to collect and resuspend cells, and transfer cell suspension to a 15 ml conical tube.
4. Spin cells down at 125×g for 5 minutes. Discard cell culture medium and resuspend in an equal volume of assay medium (Opti-MEM® I Reduced Serum Medium, no phenol red+4% FBS).
5. Count to estimate cell density and adjust density to 2×10⁵ cells/ml in assay medium.
6. Divide cells into two pools and add HaloTag® NanoBRET™ 618 Ligand or DMSO vehicle as follows: Experimental samples (+ ligand): Add 1 μl of 0.1 mM HaloTag® NanoBRET™ 618 Ligand per milliliter of cells (100 nM final concentration). No-acceptor controls (−ligand): Add 1 μl of DMSO per milliliter of cells (0.1% DMSO final concentration). Deliver compounds to the 384-well plate with Echo 550.
7. For NanoBRET™ measurements with/without compound treatment, dispense 25 μl of each pool of the cells prepared in Step 6 in duplicate to reach the requested final test concentrations (starting at 1 or 10 μM, 10-dose with 3-fold dilution).
8. Incubate plates at 37° C., 5% CO₂ 22 hours.

### Adding NanoBRET™ Nano-Glo® Substrate and Taking NanoBRET™ Measurements

1. Prepare a 5× solution of NanoBRET™ Nano-Glo® Substrate in Opti-MEM® I Reduced Serum Medium, no phenol red.
2. Add substrate to cells and shake plate to mix for 30 seconds.
3. Measure donor emission (460 nm) and acceptor emission (600 nm) within 10 minutes of substrate addition using an Envision 2104 Multilabel Reader.

### NanoBRET™ Calculations

1. Divide the acceptor emission value (e.g., 600 nm) by the donor emission value (e.g., 460 nm) for each sample to generate raw NanoBRET™ ratio values.
2. Determine the mean NanoBRET™ ratio for each set of samples: Experimental samples with HaloTag® NanoBRET™ 618 Ligand and no-acceptor control samples.
3. The IC50 curves were plotted and IC50 values were calculated using the GraphPad Prism program based on a sigmoidal dose-response equation.

### Example B-3: VCaP Growth Inhibition Assay

VCaP cells (ATCC Cat #CRL-2876) are plated at 1×10⁴ cells per well of a white plastic 96-well cluster plate (Thermo Scientific Nunc Cat #165306) in 100 uL phenol red-free DMEM (Gibco Cat #21063-029) media with 5% CSS (Omega Scientific Cat #FB-04). Cluster plates are returned to the incubator (37*C/5% CO₂) for 72 hours prior to treatment. Just prior to treatment, 100 μL of phenol red-free DMEM/5% CSS and 60 μM R1881 (Sigma Cat #R0908-10 mg) was added to each well of the cluster plates (Final R1881 concentration=30 pM). Compounds (200×) were added to each well in a volume of 1 uL, with serial dilutions (1:5) from 20 uM to 2 pM (final concentrations in well are 100 nM to 10 fM). Cluster plates are returned to the incubator for 9 days.

After the 9-day treatment period, media was removed from the cluster plates by inversion and 100 uL room temperature CTG reagent (Promega Cat #G7573) is added to each well. The cluster plates are placed on a shaker and incubated at room temperature for 20 minutes. Following this shaking/incubation, cluster plates are read on a luminometer (BMG ClarioStar). IC₅₀ values are obtained by graphing the RLU data vs. compound concentration with nonlinear regression 4-parameter logistic curves (GraphPad Prism).

Results of the VCaP growth inhibition assay are shown in Table 4.

TABLE 4

| Compound | IC₅₀** (M) |
|---|---|
| 1 | ++ |
| 2 | +++ |
| 3 | ++++ |
| 4 | ++++ |
| 5 | ++++ |
| 7 | ++ |
| 9 | ++ |
| 10 | +++ |
| 11 | ++ |
| 12 | +++ |
| 13 | ++ |
| 15 | ++++ |
| 18 | ++ |
| 19 | ++ |
| 20 | + |
| 21 | ++ |
| 22 | + |
| 24 | ++ |
| 25 | ++ |
| 26 | ++ |
| 27 | ++ |
| 28 | + |
| 41 | ++ |
| 42 | + |
| 43 | ++++ |
| 44 | ++++ |
| 45 | ++++ |
| 48 | +++ |
| 49 | +++ |
| 50 | ++ |
| 51 | ++ |
| 52 | ++ |
| 53 | ++++ |
| 54 | +++ |
| 55 | ++++ |
| 56 | ++ |
| 57 | ++++ |
| 58 | ++++ |
| 59 | ++++ |
| 60 | ++++ |
| 64 | ++ |
| 65 | + |
| 66 | ++ |
| 69 | ++ |
| 70 | ++ |
| 71 | +++ |
| 72 | ++ |
| 74 | +++ |
| 75 | ++ |
| 76 | + |
| 77 | ++ |
| 78 | +++ |
| 79 | ++ |
| 82 | + |
| 83 | ++ |
| 84 | + |
| 85 | ++ |
| 87 | ++ |
| 88 | ++ |
| 89 | ++ |
| 90 | ++++ |
| 91 | ++++ |
| 92 | ++++ |
| 93 | ++ |
| 94 | +++ |
| 95 | ++ |
| 96 | + |
| 97 | + |
| 98 | + |
| 99 | + |
| 100 | + |
| 101 | + |
| 102 | ++ |
| 103 | ++ |
| 104 | ++ |
| 105 | ++ |
| 106 | + |
| 107 | + |
| 108 | + |
| 109 | + |
| 110 | + |
| 114 | + |
| 120 | + |
| 122 | + |
| 127 | + |
| 128 | ++++ |
| 129 | + |
| 135 | + |
| 136 | + |
| 139 | + |
| 141 | + |
| 142 | + |
| 144 | ++ |
| 150 | + |
| 152 | + |
| 155 | ++ |
| 158 | + |
| 164 | + |
| 165 | + |
| 167 | ++ |
| 168 | ++ |
| 169 | + |
| 170 | ++ |
| 171 | + |
| 172 | + |
| 174 | + |
| 175 | + |
| 177 | ++++ |
| 178 | ++ |
| 179 | ++ |
| 180 | + |
| 185 | + |
| 186 | ++ |
| 188 | + |
| 190 | ++++ |
| 191 | ++++ |
| 192 | +++ |
| 194 | +++ |
| 195 | +++ |
| 196 | +++ |
| 197 | +++ |
| 198 | ++++ |
| 199 | ++++ |
| 200 | ++++ |
| 201 | ++++ |
| 202 | ++++ |
| 209 | + |
| 213 | ++ |
| 214 | +++ |
| 215 | ++ |
| 216 | + |
| 217 | ++ |
| 218 | +++ |
| 219 | ++ |
| 220 | +++ |
| 221 | +++ |
| 222 | ++ |
| 223 | + |
| 224 | ++ |
| 225 | ++ |
| 226 | + |
| 227 | + |
| 228 | ++ |
| 229 | ++ |
| 230 | ++ |
| 231 | ++ |
| 232 | ++++ |
| 233 | ++++ |
| 234 | ++++ |
| 235 | ++++ |
| 236 | ++++ |
| 237 | +++ |
| 238 | ++++ |
| 239 | +++ |
| 240 | +++ |
| 241 | ++ |
| 242 | ++ |
| 243 | ++ |
| 244 | ++ |
| 245 | ++ |
| 246 | ++ |
| 247 | ++ |
| 248 | ++ |
| 249 | ++ |
| 250 | + |
| 251 | ++ |
| 252 | ++ |
| 253 | + |
| 254 | ++ |
| 255 | ++ |
| 256 | ++ |
| 257 | ++ |
| 263 | + |
| 264 | ++ |
| 265 | + |
| 266 | ++ |
| 267 | +++ |
| 268 | ++ |
| 269 | ++ |
| 270 | ++ |
| 271 | ++ |
| 273 | + |
| 274 | + |
| 275 | + |
| 277 | + |
| 284 | + |
| 286 | +++ |
| 287 | +++ |
| 288 | + |
| 289 | + |
| 292 | ++ |
| 293 | ++ |
| 294 | ++ |
| 296 | ++ |
| 297 | +++ |
| 298 | ++++ |
| 299 | ++ |
| 300 | + |
| 301 | + |
| 302 | + |
| 304 | ++ |
| 305 | + |
| 306 | +++ |
| 308 | + |
| 309 | ++ |
| 310 | + |
| 313 | ++ |
| 317 | + |
| 318 | + |
| 319 | + |
| 320 | ++ |
| 321 | + |
| 322 | + |
| 323 | + |
| 324 | + |
| 325 | ++ |
| 327 | ++ |
| 329 | + |
| 331 | ++ |
| 332 | + |
| 333 | + |
| 334 | ++ |
| 335 | ++ |
| 336 | +++ |
| 337 | ++ |
| 338 | ++ |
| 339 | + |
| 341 | ++ |
| 342 | ++ |
| 343 | ++ |
| 347 | + |
| 348 | + |
| 349 | + |
| 350 | + |
| 356 | + |
| 357 | + |
| 360 | + |
| 362 | + |
| 363 | + |
| 364 | + |
| 369 | + |
| 371 | + |
| 372 | + |
| 373 | + |
| 374 | ++ |
| 375 | ++ |
| 376 | ++ |
| 377 | +++ |
| 378 | +++ |
| 379 | +++ |
| 380 | +++ |
| 383 | ++ |
| 384 | + |
| 385 | ++ |
| 386 | ++ |
| 387 | ++ |
| 388 | ++ |
| 389 | + |
| 390 | ++ |
| 392 | ++ |
| 395 | ++ |
| 396 | ++ |
| 397 | ++++ |
| 398 | +++ |
| 399 | +++ |
| 401 | ++ |
| 402 | + |
| 406 | +++ |
| 410 | ++++ |
| 412 | ++++ |
| 415 | +++ |
| 432 | + |
| 442 | + |
| 443 | + |
| 444 | ++ |
| 446 | ++ |
| 447 | ++ |
| 448 | ++ |
| 449 | ++ |
| 450 | ++ |
| 451 | ++ |
| 455 | ++ |
| 458 | ++ |
| 459 | ++ |
| 460 | + |
| 461 | + |
| 462 | + |
| 464 | + |
| 465 | ++ |
| 466 | ++ |
| 468 | ++ |
| 471 | ++ |
| 472 | ++ |
| 473 | ++ |

**++++ is IC₅₀ active and >1E−7; +++ is 1E−7 ≤ IC₅₀ > 1E−9; ++ is 1E−9 ≤ IC₅₀ > 1E−10; + is IC₅₀ ≤ 1E−10

### Example B-4: HEK-293 Growth Inhibition Assay

HEK-293 cells (ATCC Cat #CRL-1573) are plated at 3×10³ cells per well of a white plastic 96-well cluster plate (Thermo Scientific Nunc Cat #165306) in 200 uL DMEM (ATCC Cat #30-2002) media with 10% FBS (Gibco Cat #26140-079). Cluster plates are returned to the incubator (37*C/5% CO2) for 24 hours prior to treatment. Compounds (200×) were added to each well in a volume of 1 uL, with serial dilutions (1:5) from 2 mM to 200 pM (final concentrations in well are 10 uM to 1 pM). Cluster plates are returned to the incubator for 3 days.

After the 3-day treatment period, 100 uL media was removed from each well by aspiration and 100 uL room temperature CTG reagent (Promega Cat #G7573) is added back to each well. The cluster plates are placed on a shaker and incubated at room temperature for 20 minutes. Following this shaking/incubation, cluster plates are read on a luminometer (BMG ClarioStar). IC₅₀ values are obtained by graphing the RLU data vs. compound concentration with nonlinear regression 4-parameter logistic curves (GraphPad Prism).

Results of the HEK-293 growth inhibition assay are shown in Table 5.

TABLE 5

| Compound | IC₅₀ (μM) |
|---|---|
| 20 | >10 |
| 22 | >10 |
| 28 | >10 |
| 41 | >10 |
| 42 | >10 |
| 57 | >10 |
| 59 | >10 |
| 64 | >10 |
| 65 | >10 |
| 66 | >10 |
| 69 | >10 |
| 70 | >10 |
| 72 | >10 |
| 74 | >10 |
| 75 | >10 |
| 77 | >10 |
| 79 | >10 |
| 82 | >10 |
| 83 | >10 |
| 84 | >10 |
| 85 | >10 |
| 87 | >10 |
| 90 | 2.159 |
| 91 | >10 |
| 96 | >10 |
| 97 | >10 |
| 98 | >10 |
| 99 | >10 |
| 100 | >10 |
| 101 | >10 |
| 102 | >10 |
| 103 | >10 |
| 104 | >10 |
| 105 | >10 |
| 106 | >10 |
| 107 | >10 |
| 108 | >10 |
| 109 | >10 |
| 110 | >10 |
| 172 | >10 |
| 263 | >1 |
| 273 | >1 |
| 275 | >1 |
| 327 | >1 |
| 350 | >1 |
| 356 | >1 |
| 360 | >1 |

What is claimed is:

1. A heterobifunctional conditional inhibitor compound of Formula (I), or a pharmaceutically acceptable salt or solvate thereof: 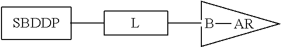
wherein:
SBDDP is a silent binder of a disease-dependent protein (DDP);
L is an optional linker; wherein L is covalently attached at a position of SBDDP that is solvent exposed when SBDDP binds to the DDP;
B-AR is a binder of the androgen receptor (AR), wherein B-AR comprises:
1) a head group that occupies the ligand-binding domain (LBD) of AR and is covalently attached to a core moiety; and
2) an optional tail moiety covalently attached to the core moiety; wherein the core comprises an optionally substituted cyclobutyl having the structure of Formula (C): 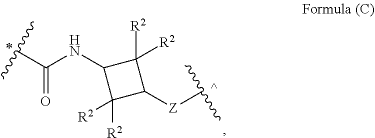
the head group is covalently attached to Z at position (\hat{ });
the optional tail moiety comprises a ring D that is covalently attached to the amide (*), wherein ring D is a 5-, 6-, 8-, 9- or 10-membered aryl or a 5-, 6-, 8-, 9- or 10-membered heteroaryl that is optionally substituted with s R³;
wherein:
Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈ cycloalkyl or a substituted or unsubstituted monocyclic 3- to 8-membered heterocycloalkyl;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle; and
each s is 1, 2, or 3;
wherein L is covalently attached to the core moiety at position (*) if the optional tail moiety is absent, or L is covalently attached to the tail moiety if present;
wherein DDP and AR are both expressed in a cell of interest (COI), and wherein the relative abundance of the AR in the COI is greater than the relative abundance of the DDP in the COI; or the COI is a diseased cell, and the AR is overexpressed, overactive, or both overexpressed and overactive, or amplified in the diseased cell as compared to when the COI is a non-diseased cell.

2. The compound of claim 1, or a pharmaceutically acceptable salt or solvate thereof, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

3. The compound of claim 1 or claim 2, or a pharmaceutically acceptable salt or solvate thereof, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

4. The compound of any one of claims 1-3, or a pharmaceutically acceptable salt or solvate thereof, wherein DDP is CREB-binding protein (CBP)/p300.

5. The compound of claim 4, or a pharmaceutically acceptable salt or solvate thereof, wherein SBDDP binds in the acetyl-lysine (KAc) binding site of the bromodomain of CBP/p300.

6. The compound of claim 4, or a pharmaceutically acceptable salt or solvate thereof, wherein the SBDDP comprises an acetyl-lysine mimetic moiety that binds in the acetyl-lysine (KAc) binding site of the bromodomain of CBP/p300.

7. The compound of claim 6, or a pharmaceutically acceptable salt or solvate thereof, wherein:
the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300; and
the head group of B-AR forms hydrogen bonds with the side chains of Gln 711 and Arg752 of the LBD of AR.

8. The compound of any one of claims 1-7, or a pharmaceutically acceptable salt or solvate thereof, wherein:
SBDDP further comprises a moiety that interacts with Arg1173 in the bromodomain of CBP or Asn1137 in the bromodomain of p300.

9. The compound of any one of claims 1-8, or a pharmaceutically acceptable salt or solvate thereof, wherein SBDDP further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2).

10. The compound of any one of claims 6-9, or a pharmaceutically acceptable salt or solvate thereof, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

11. The compound of claim 9 or 10, or a pharmaceutically acceptable salt or solvate thereof, wherein L is covalently attached to SBDDP on:
the acetyl-lysine mimetic moiety; or
the moiety that occupies the lipophilic shelf (LPF) region of the bromodomain of CBP/p300, if present; or
the moiety that occupies the BC Loop region of the bromodomain of CBP/p300, if present;
wherein L is covalently attached to SBDDP at a position that does not interfere with the binding of the acetyl-lysine mimetic moiety in the acetylated lysine (KAc) binding site of CBP/p300.

12. The compound of any one of claims 1-11, or a pharmaceutically acceptable salt or solvate thereof, wherein:
the head group of B-AR comprises 4-cyanophenyl; 3-fluoro-4-cyanophenyl; 3-chloro-4-cyanophenyl; 3-methoxy-4-cyanophenyl; 3-methyl-4-cyanophenyl; 3-trifluroromethyl-4-cyanophenyl; 3-trifluroromethoxy-4-cyanophenyl; 5-fluoro-6-cyanopyridin-3-yl; 5-chloro-6-cyanopyridin-3-yl; 5-methoxy-6-cyanopyridin-3-yl; 5-methyl-6-cyanopyridin-3-yl; 5-trifluroromethyl-6-cyanopyridin-3-yl; 5-trifluroromethoxy-6-cyanopyridin-3-yl; [1,2,4]triazolo[4,3-b]pyridazin-6-yl; or 3-(trifluoromethyl)-[1,2,4]triazolo[4,3-b]pyridazin-6-yl; and
each R² is —CH₃.

13. The compound of any one of claims 1-12, or a pharmaceutically acceptable salt or solvate thereof, wherein the acetyl-lysine mimetic moiety that binds in the acetyl-lysine (KAc) binding site of the bromodomain of CBP/p300 comprises:
1-(1,4,6,7-tetrahydro-5H-pyrazolo[4,3-c]pyridin-5-yl)ethan-1-one; or N-methyl-1,4,6,7-tetrahydro-5H-pyrazolo[4,3-c]pyridine-5-carboxamide; wherein the acetyl-lysine mimetic moiety optionally further comprises:
1) a moiety at the 1-position of the 1,4,6,7-tetrahydro-5H-pyrazolo[4,3-c]pyridin-5-yl group that occupies the lipophilic shelf (LPF) region of the bromodomain of CBP/p300; or
2) a moiety that at the 3-position of the 1,4,6,7-tetrahydro-5H-pyrazolo[4,3-c]pyridin-5-yl group occupies the BC Loop region of the bromodomain of CBP/p300; or
3) both 1) and 2).

14. A heterobifunctional conditional inhibitor compound of Formula (II), or a pharmaceutically acceptable salt or solvate thereof: 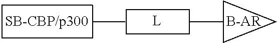
wherein:
SB-CBP/p300 is a silent binder of human CREB-binding protein (CBP) or human E1A-binding protein p300 (p300) (CBP/p300);
L is an optional linker; wherein L is covalently attached at a position of SB-CBP/p300 that is solvent exposed when SB binds to CBP/p300;
B-AR is a binder of the androgen receptor (AR), wherein the B-AR comprises:
1) ahead group that occupies the ligand-binding domain (LBD) of AR and is covalently attached to a core moiety; and
2) an optional tail moiety covalently attached to the core moiety; wherein the core comprises an optionally substituted cyclobutyl having the structure of Formula (C): 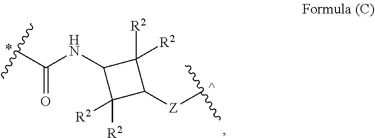
wherein:
the head group is covalently attached to Z at position (\hat{ });
the optional tail moiety comprises a ring D that is covalently attached to the amide (*), wherein ring D is a 5-, 6-, 8-, 9- or 10-membered aryl or a 5-, 6-, 8-, 9- or 10-membered heteroaryl that is optionally substituted with s R³;
Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈cycloalkyl or a substituted or unsubstituted monocyclic 3- to 8-membered heterocycloalkyl;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle; and
each s is 1, 2, or 3;
wherein L is covalently attached to the core moiety at position (*) if the optional tail moiety is absent, or L is covalently attached to the tail moiety if present.

15. The compound of any one of claims 1-11 or 14, or a pharmaceutically acceptable salt or solvate thereof, wherein:
the head group of B-AR forms hydrogen bonds with the side chains of Gln 711 and Arg752 of the LBD of AR; and each R² is independently hydrogen, —CH₃, —CD₃, —CH₂F, —CHF₂, or —CF₃.

16. The compound of any one of claims 1-11 or 14, or a pharmaceutically acceptable salt or solvate thereof, wherein: each R² is —CH₃.

17. The compound of any one of claims 1-11 or 14, or a pharmaceutically acceptable salt or solvate thereof, wherein B-AR comprises a head group and an optional core, wherein the head group is selected from: 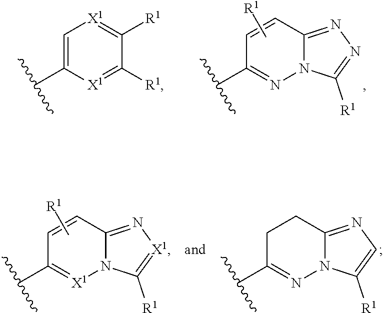
each X¹ is independently —CR¹— or —N—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂.

18. The compound of any one of claims 1-11 or 14, or a pharmaceutically acceptable salt or solvate thereof, wherein B-AR comprises a head group and an optional core, wherein the head group is selected from: 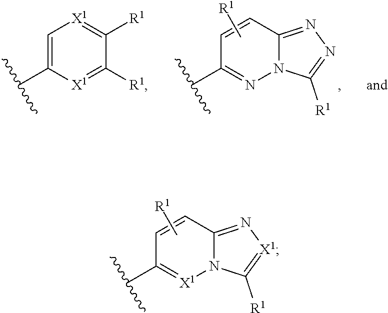
one X¹ is —CR¹— and the other X¹ is —CR¹— or —N—;
each R¹ is independently hydrogen, F, Cl, Br, I, —CH₃, —CD₃, —CH₂CH₃, —CH₂F, —CHF₂, —CF₃, —CH₂CH₂F, —CH₂CHF₂, —CH₂CF₃, —OH, —OCF₃, —OCH₃, —OCD₃, —OCH₂CH₃, —CN, —C(═O)NH₂—C(═O)NH(CH₃) or —C(═O)NH(CD₃).

19. The compound of any one of claims 1-11 or 14, or a pharmaceutically acceptable salt or solvate thereof, wherein B-AR comprises a head group and an optional core, wherein the head group is selected from: 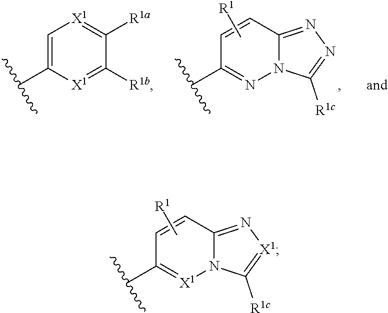
each X¹ is independently —CR¹— or —N—;
R^{1a} is —CN, —NO₂, —C(═O)R⁵, —C(═O)OR⁵, or —C(═O)N(R⁵)₂;
R^{1b} is hydrogen, halogen, C₁-C₄alkyl, C₁-C₄fluoroalkyl, —CN, —OH, —OR⁴, or —SR⁴;
R^{1c} is hydrogen, halogen, C₁-C₄alkyl, C₁-C₄fluoroalkyl, or —CN;
each R¹ is independently hydrogen, halogen, C₁-C₄alkyl, C₁-C₄fluoroalkyl, —CN, —OH, or —OR⁴;
each R⁴ is independently C₁-C₄alkyl, or C₁-C₄fluoroalkyl;
each R⁵ is independently hydrogen, C₁-C₄alkyl, or C₁-C₄fluoroalkyl.

20. The compound of any one of claims 1-11 or 14, or a pharmaceutically acceptable salt or solvate thereof, wherein B-AR comprises a head group and an optional core, wherein the head group is selected from: 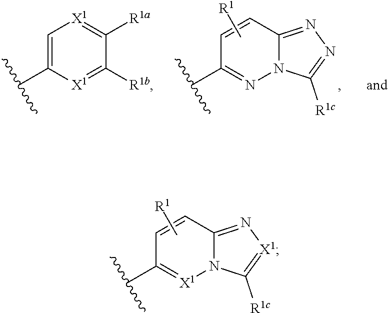
one X¹ is —CR¹— and the other X¹ is —CR¹— or —N—;
R^{1a} is —CN, —NO₂, —C(═O)NH₂ or —C(═O)NH(CH₃);
R^{1b} is hydrogen, F, Cl, Br, I, —CH₃, —CH₂CH₃, —CH₂F, —CHF₂, —CF₃, —OH, —OCF₃, —OCH₃, —OCH₂CH₃, or —CN;
each R^{1c} is hydrogen, F, Cl, Br, —CH₃, —CH₂F, —CHF₂, or —CF₃;
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, or —OCH₂CH₃.

21. The compound of any one of claims 1-11 or 14, or a pharmaceutically acceptable salt or solvate thereof, wherein B-AR comprises a head group and an optional core, wherein the head group is selected from:

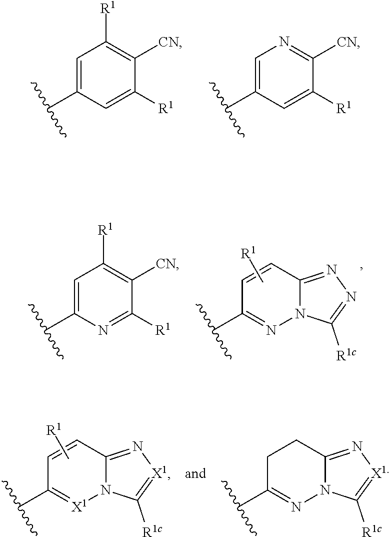

22. The compound of any one of claims 1-11 or 14, or a pharmaceutically acceptable salt or solvate thereof, wherein B-AR comprises a head group and an optional core, wherein the head group is selected from:

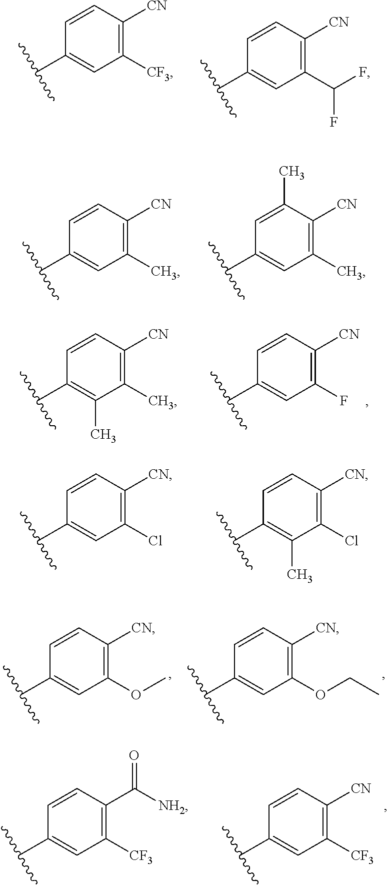

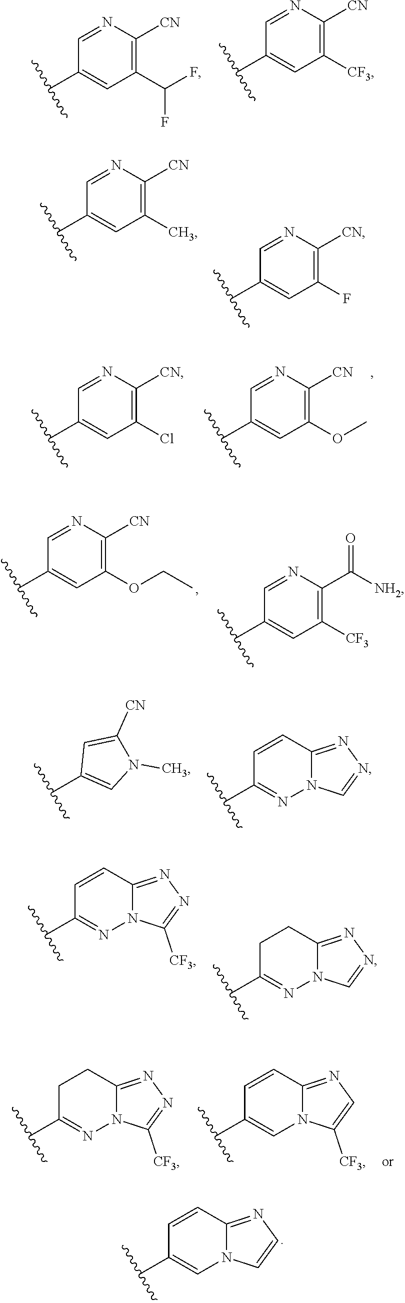

23. The compound of any one of claim 1-11 or 14-20, or a pharmaceutically acceptable salt or solvate thereof, wherein B-AR has the structure of Formula (IIa), or a pharmaceutically acceptable salt or solvate thereof: 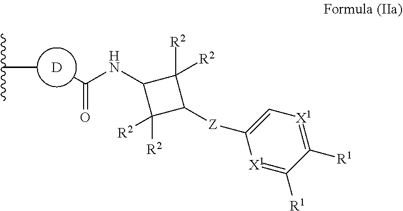
wherein:
each X¹ is independently —CR¹— or —N—;
Z is —O— or —NR⁵—;
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂ or —C(═O)NTH(CH₃);
each R² is C₁-C₆alkyl;
ring D that is a 5-, 6-, 8-, 9- or 10-membered aryl or a 5-, 6-, 8-, 9- or 10-membered heteroaryl that is optionally substituted with R³;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂;
s is 1, 2, or 3.

24. The compound of claim 23, or a pharmaceutically acceptable salt or solvate thereof, or a pharmaceutically acceptable salt or solvate thereof:
one X¹ is —CR¹— and the other X¹ is —CR¹— or —N—;
each R² is —CH₃.

25. The compound of any one of claims 1-11 or 14-20, or a pharmaceutically acceptable salt or solvate thereof, wherein B-AR has the structure of Formula (IIIa), or a pharmaceutically acceptable salt or solvate thereof: 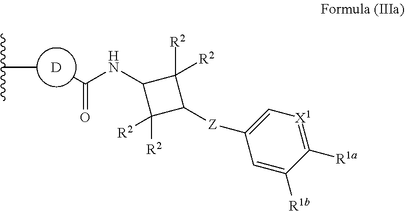
wherein:
X¹ is —CR¹— or —N—;
Z is —O— or —NR⁵—;
R^{1a} is —CN, —NO₂, —C(═O)R⁵, —C(═O)OR⁵, or —C(═O)N(R⁵)₂;
R^{1b} is hydrogen, halogen, C₁-C₄alkyl, C₁-C₄fluoroalkyl, —CN, —OH, or —OR⁴;
each R¹ is independently hydrogen, halogen, C₁-C₄alkyl, C₁-C₄fluoroalkyl, —CN, —OH, or —OR⁴;
each R² is —CH₃;
ring D that is a 5-, 6-, 8-, 9- or 10-membered aryl or a 5-, 6-, 8-, 9- or 10-membered heteroaryl that is optionally substituted with s R³;
each R³ is independently hydrogen, halogen, substituted or unsubstituted C₁-C₄alkyl, C₁-C₄fluoroalkyl, —CN, —OH, —OR⁴, or —N(R⁵)₂;
s is 1, 2, or 3;
each R⁴ is independently C₁-C₄alkyl, or C₁-C₄fluoroalkyl;
each R⁵ is independently hydrogen, C₁-C₄alkyl, or C₁-C₄fluoroalkyl.

26. The compound of any one of claims 23-25, or a pharmaceutically acceptable salt or solvate thereof, wherein ring D is phenyl, naphthyl, pyridinyl, pyrimidinyl, pyridazinyl, pyrazinyl, triazinyl, pyrrolyl, thiazolyl, oxazolyl, isoxazolyl, oxadiazolyl, imidazolyl, pyrazolyl, isothiazolyl, triazolyl, and tetrazolyl, wherein each ring D is optionally substituted with s R³.

27. The compound of any one of claims 23-25, or a pharmaceutically acceptable salt or solvate thereof,
wherein ring D is 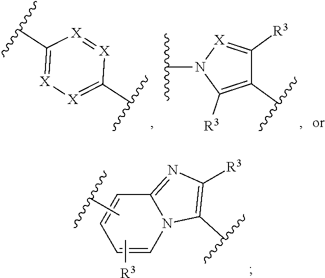
and each X is independently —CR³— or —N—.

28. The compound of any one of claims 23-25, or a pharmaceutically acceptable salt or solvate thereof, wherein ring D is

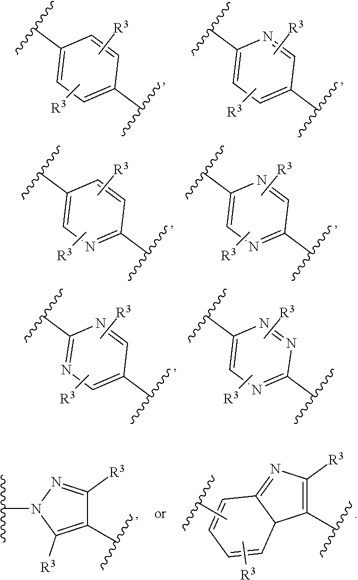

29. The compound of any one of claims 1-11 or 14-20, or a pharmaceutically acceptable salt or solvate thereof, wherein B-AR has the structure of Formula (IVa), or a pharmaceutically acceptable salt or solvate thereof: 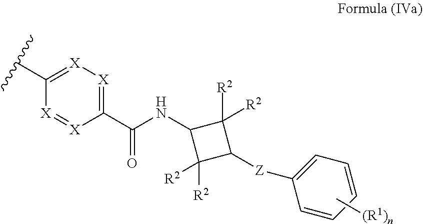
wherein:
each X is independently —CR³— or —N—;
Z is —O— or —NR⁵—;
n is 2 and one R¹ is halogen, —CH₃, —OCH₃, or —CF₃, and the other R¹ is —CN;
each R² is —CH₃.

30. The compound of claim 20, or a pharmaceutically acceptable salt or solvate thereof, wherein:
one R¹ is —Cl, —CH₃, —OCH₃, or —CF₃, and the other R¹ is —CN.

31. The compound of any one of claims 1-11 or 14-20, or a pharmaceutically acceptable salt or solvate thereof, wherein B-AR has the structure of Formula (Va), or a pharmaceutically acceptable salt or solvate thereof: 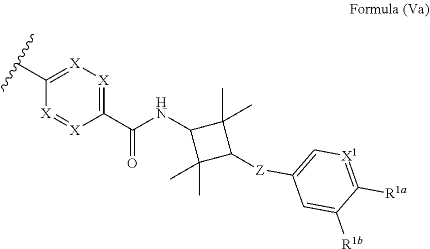
wherein:
each X is independently —CR³— or —N—;
Z is —O— or —NR⁵—;
X¹ is —CR¹— or —N—;
R^{1a} is —CN;
R^{1b} is hydrogen, F, Cl, Br, I, —CH₃, —CH₂CH₃, —CH₂F, —CHF₂, —CF₃, —OH, —OCF₃, —OCH₃, or —CN;
R¹ is hydrogen, F, Cl, Br, I, —CH₃, —CH₂CH₃, —CH₂F, —CHF₂, —CF₃, —CH₂CF₃, —OH, —OCF₃, —OCH₃, —OCH₂CH₃, or —OCH₂CF₃;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂;
each R⁴ is independently substituted or unsubstituted C₁-C₆alkyl, or C₁-C₆fluoroalkyl;
each R⁵ is independently hydrogen, substituted or unsubstituted C₁-C₆alkyl, C₁-C₆fluoroalkyl.

32. The compound of any one of claims 29-31, or a pharmaceutically acceptable salt or solvate thereof, wherein

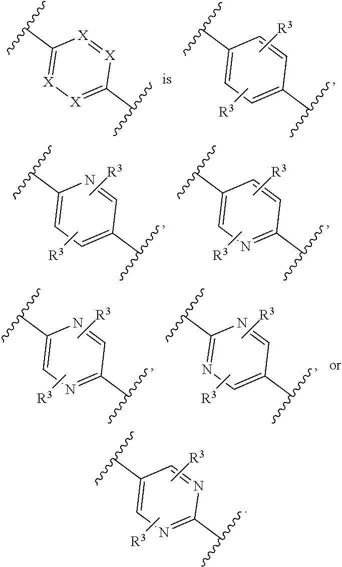

33. The compound of any one of claims 1-32, or a pharmaceutically acceptable salt or solvate thereof, wherein:
each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂, or —C(═O)NH(CH₃).

34. The compound of any one of claims 29-33, or a pharmaceutically acceptable salt or solvate thereof, wherein:

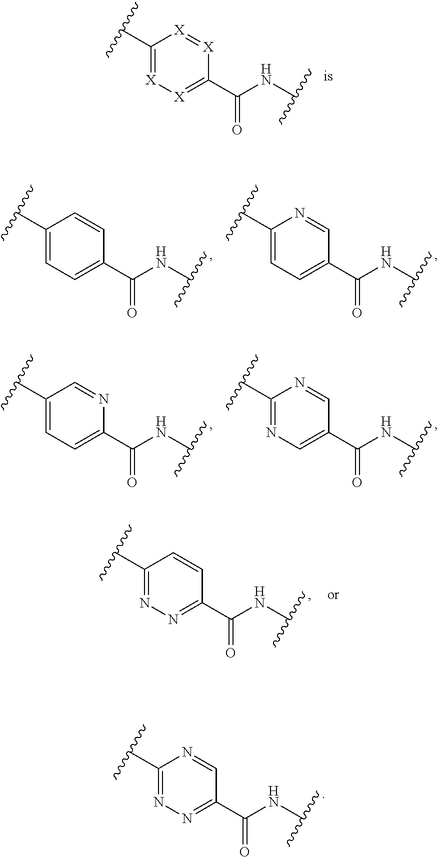

35. The compound of any one of claims 1-11 or 14, or a pharmaceutically acceptable salt or solvate thereof, wherein the head group and core of B-AR is:
N-(3-(3-fluoro-4-cyanophenoxy)-2,2,4,4-tetramethylcyclobutyl)acetamide-*;
N-(3-(3-chloro-4-cyanophenoxy)-2,2,4,4-tetramethylcyclobutyl)acetamide-*;
N-(3-(3-methoxy-4-cyanophenoxy)-2,2,4,4-tetramethylcyclobutyl)acetamide-*;
N-(3-(3-methyl-4-cyanophenoxy)-2,2,4,4-tetramethylcyclobutyl)acetamide-*;
N-(3-(3-trifluroromethyl-4-cyanophenoxy)-2,2,4,4-tetramethylcyclobutyl)acetamide-*;
N-(3-(3-trifluroromethoxy-4-cyanophenoxy)-2,2,4,4-tetramethylcyclobutyl)acetamide-*;
N-(3-((5-fluoro-6-cyanopyridin-3-yl)oxy)-2,2,4,4-tetramethylcyclobutyl)acetamide-*;
N-(3-((5-chloro-6-cyanopyridin-3-yl)oxy)-2,2,4,4-tetramethylcyclobutyl)acetamide-*;
N-(3-((5-methoxy-6-cyanopyridin-3-yl)oxy)-2,2,4,4-tetramethylcyclobutyl)acetamide-*;
N-(3-((5-methyl-6-cyanopyridin-3-yl)oxy)-2,2,4,4-tetramethylcyclobutyl)acetamide-*;
N-(3-((5-trifluroromethyl-6-cyanopyridin-3-yl)oxy)-2,2,4,4-tetramethylcyclobutyl)acetamide-*; or
N-(3-((5-trifluroromethoxy-6-cyanopyridin-3-yl)oxy)-2,2,4,4-tetramethylcyclobutyl)acetamide-*;
wherein acetamide-* is

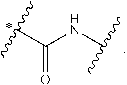 [SMILES: *NC(*)=O]

36. The compound of any one of claims 1-11 or 14, or a pharmaceutically acceptable salt or solvate thereof, wherein the head group and core of B-AR is:
N-(3-(3-chloro-4-cyanophenoxy)-2,2,4,4-tetramethylcyclobutyl)acetamide-*;
N-(3-(3-methoxy-4-cyanophenoxy)-2,2,4,4-tetramethylcyclobutyl)acetamide-*;
N-(3-(3-methyl-4-cyanophenoxy)-2,2,4,4-tetramethylcyclobutyl)acetamide-*; or
N-(3-(3-trifluroromethyl-4-cyanophenoxy)-2,2,4,4-tetramethylcyclobutyl)acetamide-*;
wherein acetamide-* is

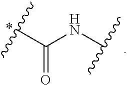 [SMILES: *NC(*)=O]

37. The compound of claim 35 or 36, or a pharmaceutically acceptable salt or solvate thereof, wherein B-AR further comprises a tail moiety that is covalently attached to position (*), wherein the tail moiety is a ring D that is phenyl, pyrimidinyl, pyrazinyl, pyridazinyl, triazinyl, triazolyl, pyrrolyl, imidazolyl, pyrazolyl, pyridyl, indolyl, isoindolyl, indolizinyl, benzimidazolyl, napthyl, quinolyl, isoquinolyl, quinoxalinyl, quinazolinyl, indazolyl, or benzotriazolyl; wherein ring D is optionally substituted with s R³;
s is 1, 2, or 3;
each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂, or —C(═O)NH(CH₃).

38. The compound of claim 35 or 36, or a pharmaceutically acceptable salt or solvate thereof, wherein ring D is phenyl, pyridyl, pyrimidinyl, pyrazinyl, pyridazinyl, or triazinyl; wherein ring D is optionally substituted with s R³; s is 1, 2, or 3;
each R³ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, or —CN.

39. The compound of claim 1-11 or 14, or a pharmaceutically acceptable salt or solvate thereof, wherein B-AR has one of the following structures:

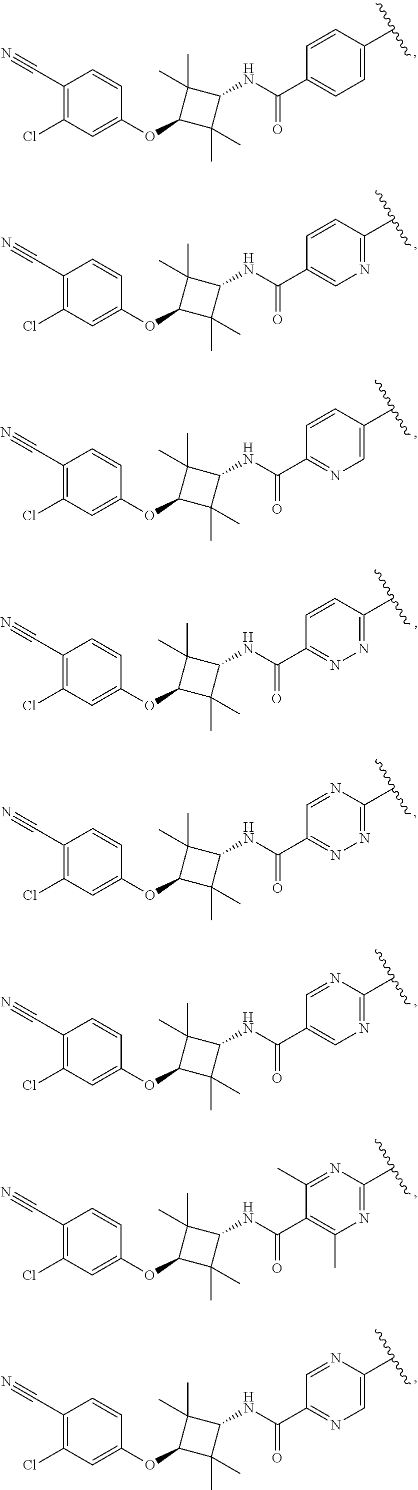

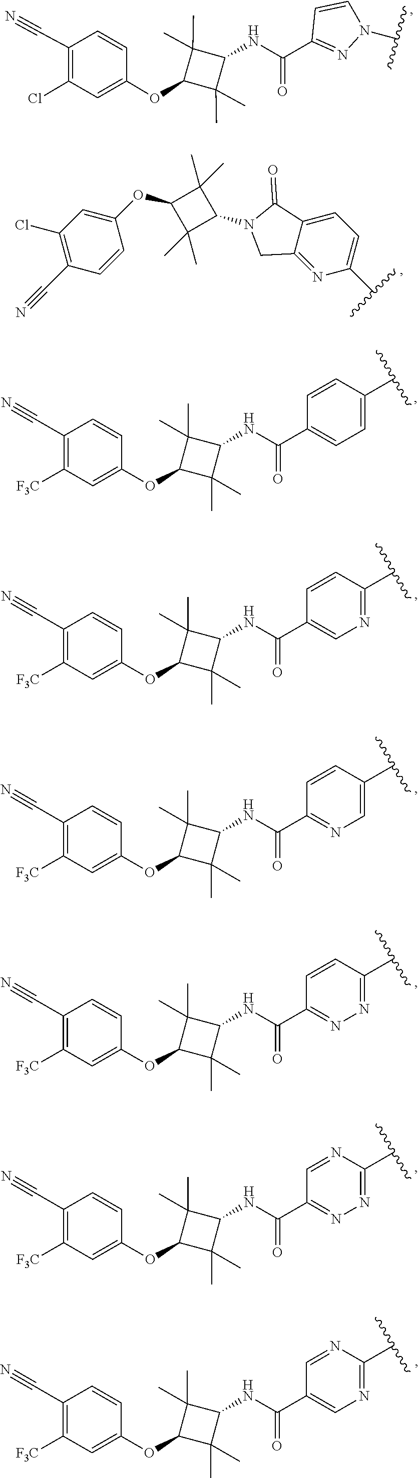

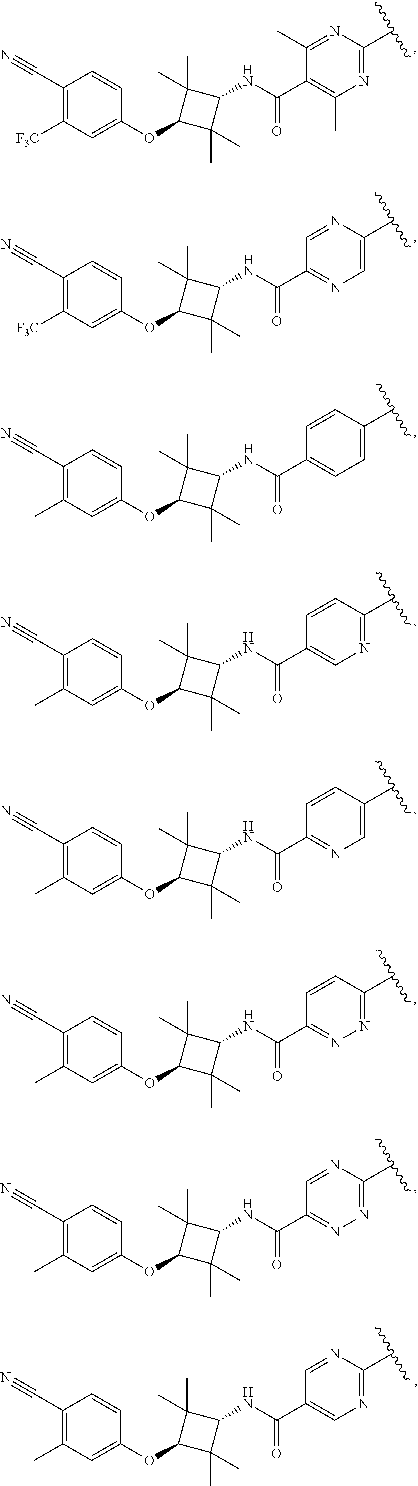

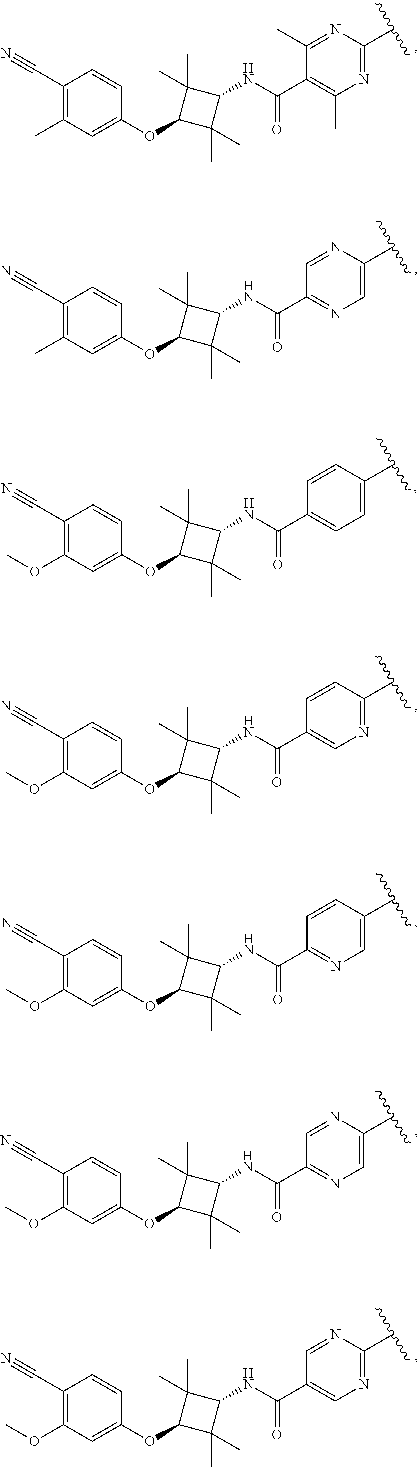

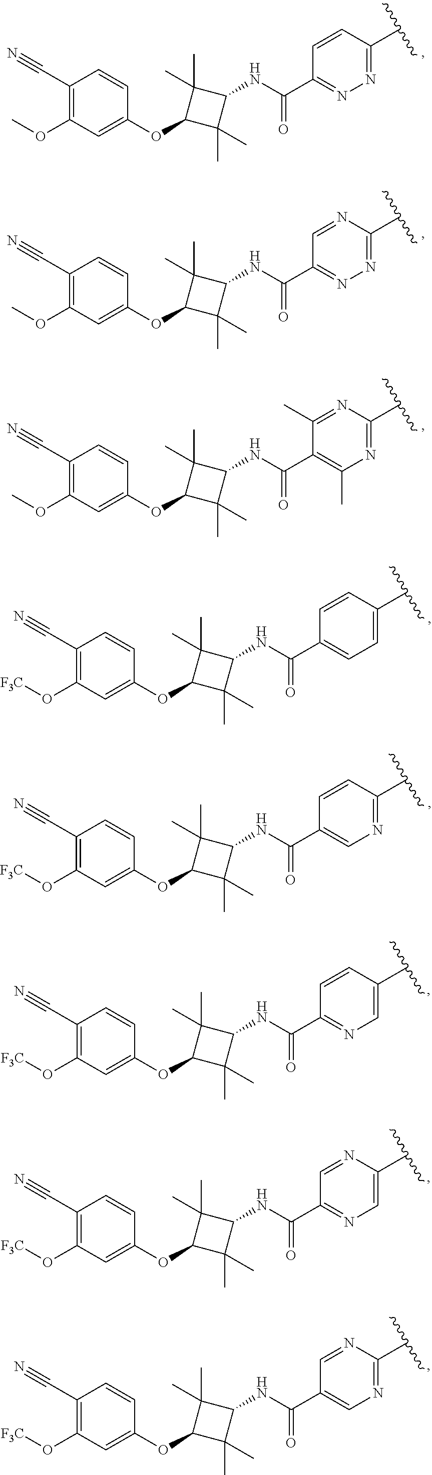

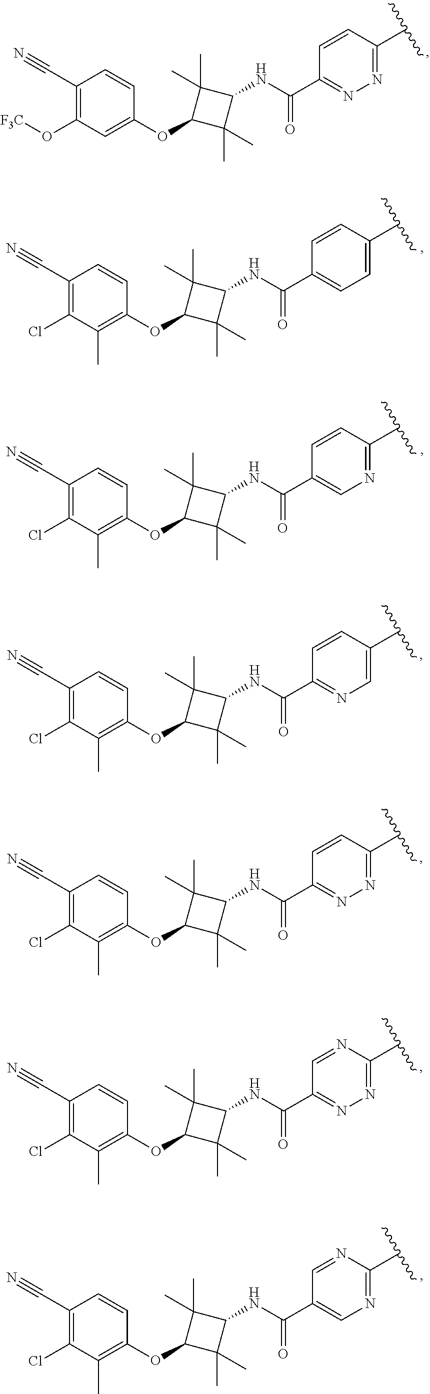

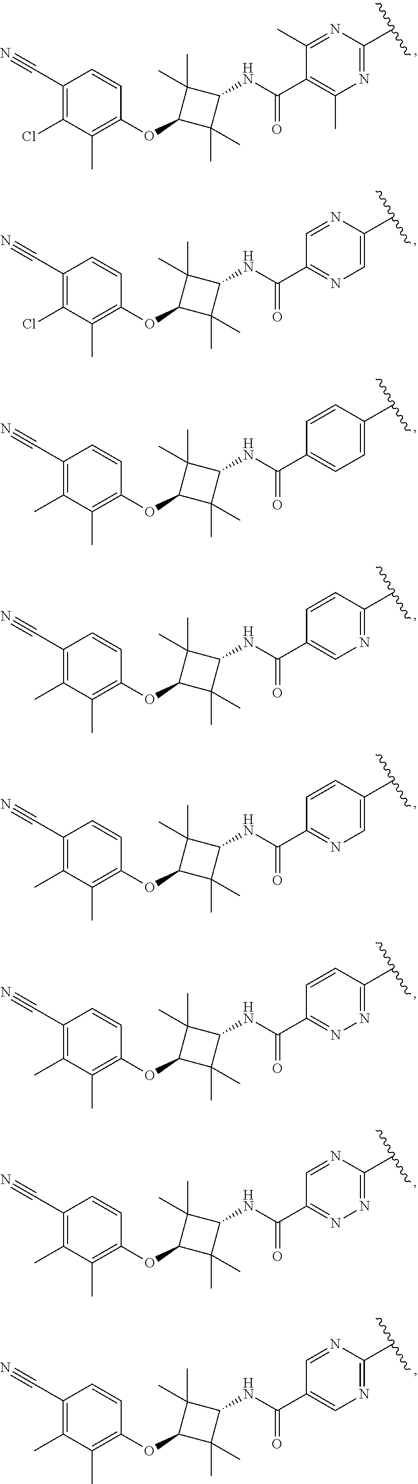

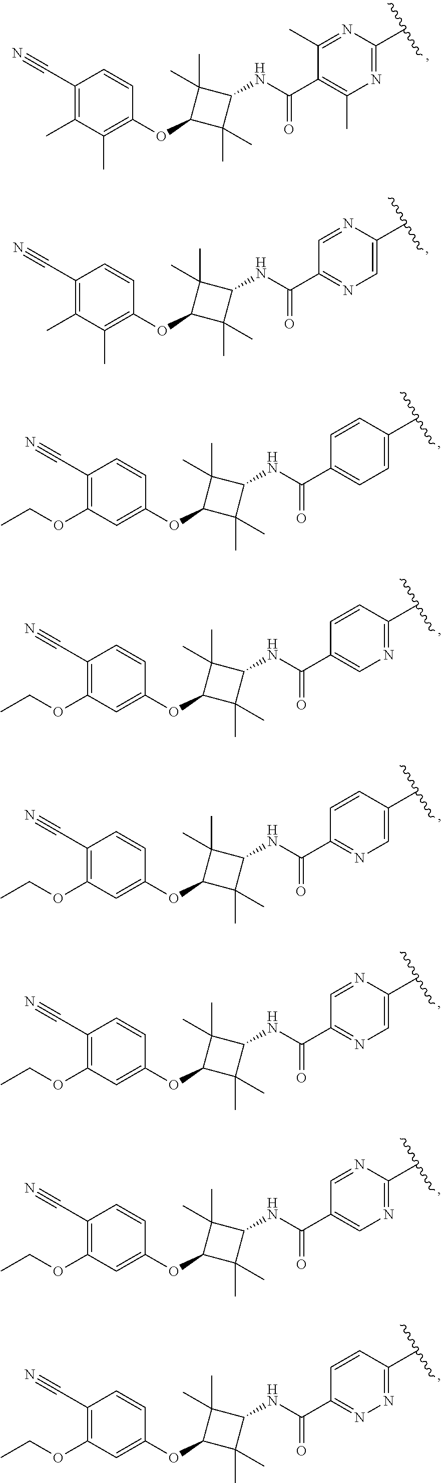

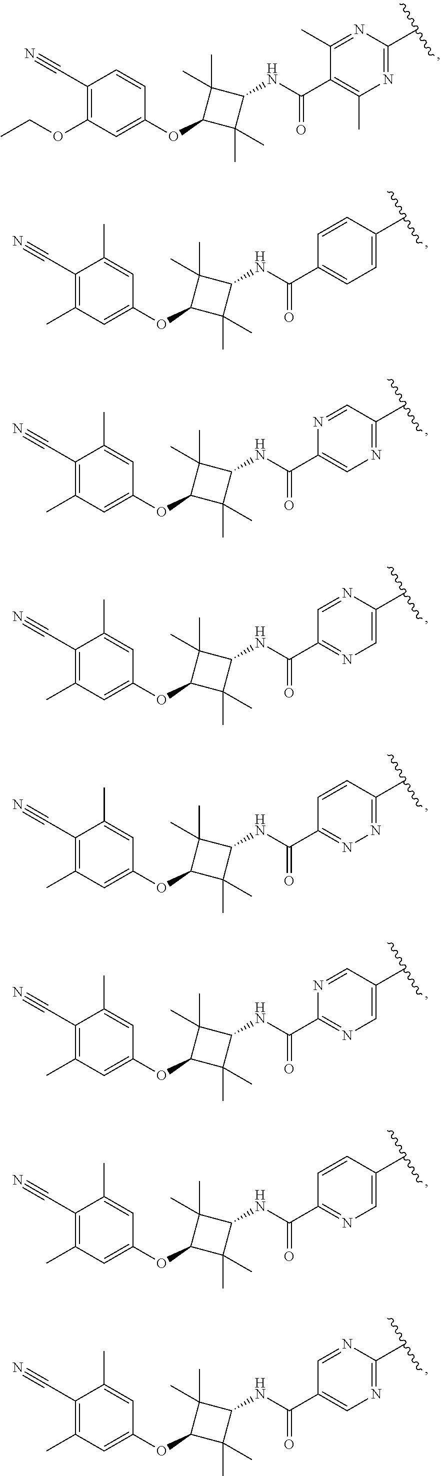

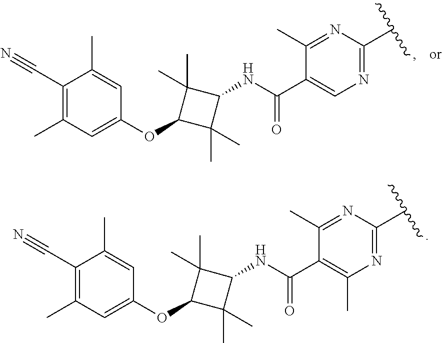 [SMILES: *c1ncc(C(=O)N[C@H]2C(C)(C)[C@H](Oc3cc(C)c(C#N)c(C)c3)C2(C)C)c(C)n1 ; *c1nc(C)c(C(=O)N[C@H]2C(C)(C)[C@H](Oc3cc(C)c(C#N)c(C)c3)C2(C)C)c(C)n1]

40. The compound of any one of claims 14-39, or a pharmaceutically acceptable salt or solvate thereof, wherein SB-CBP/p300 binds in the acetyl-lysine (KAc) binding site of the bromodomain of CBP/p300.

41. The compound of any one of claims 14-39, or a pharmaceutically acceptable salt or solvate thereof, wherein SB-CBP/p300 comprises an acetyl-lysine mimetic moiety that binds in the acetyl-lysine (KAc) binding site of the bromodomain of CBP/p300.

42. The compound of claim 41, or a pharmaceutically acceptable salt or solvate thereof, wherein L is covalently attached at a position of a) that is solvent exposed when a) binds the KAc binding site of the bromodomain of CBP/p300.

43. The compound of claim 41, or a pharmaceutically acceptable salt or solvate thereof, wherein the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300; and the head group of B-AR forms hydrogen bonds with the side chains of Gln 711 and Arg 752 of the LBD of AR.

44. The compound of any one of claims 14-43, or a pharmaceutically acceptable salt or solvate thereof, wherein SB-CBP/p300 further comprises a moiety that interacts with Arg1173 in the bromodomain of CBP or Asn1137 in the bromodomain of p300.

45. The compound of any one of claims 14-44, or a pharmaceutically acceptable salt or solvate thereof, wherein SB-CBP/p300 further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2).

46. The compound of claim 41-45, or a pharmaceutically acceptable salt or solvate thereof, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

47. The compound of claim 45 or 46, or a pharmaceutically acceptable salt or solvate thereof, wherein L is covalently attached to SB-CBP/p300 on:
the acetyl-lysine mimetic moiety; or
the moiety that occupies the lipophilic shelf (LPF) region of the bromodomain of CBP/p300, if present; or
the moiety that occupies the BC Loop region of the bromodomain of CBP/p300, if present;
wherein L is covalently attached to SB-p300/CBP at a position that does not interfere with the binding of the acetyl-lysine mimetic moiety in the acetylated lysine (KAc) binding site of CBP/p300.

48. The compound of any one of claims 1-47, or a pharmaceutically acceptable salt or solvate thereof, wherein the moiety comprising an acetyl-lysine mimetic comprises a moiety selected from: 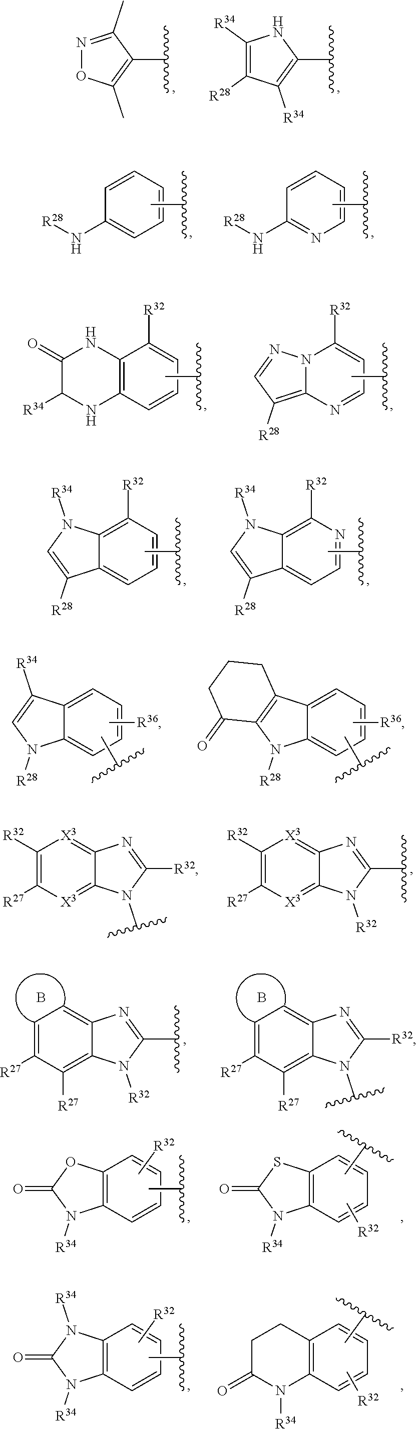 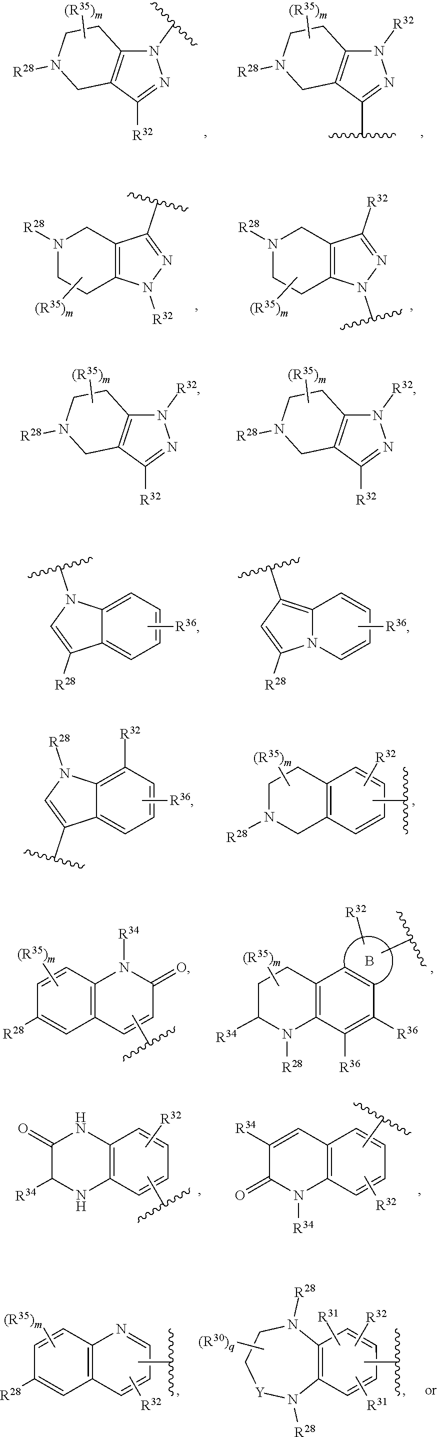 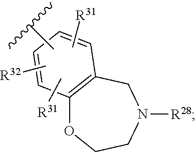
each R³² is independently an optional moiety that occupies the lipophilic shelf (LPF) region of the bromodomain of CBP/p300;
or each R³² is independently an optional moiety that occupies the BC Loop region of the bromodomain of CBP/p300; 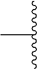
is the point of attachment to L that covalently connects SB-CBP/p300 to B-AR;
or R³² comprises 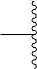
and L that covalently connects SB-CBP/p300 to B-AR is attached to R³²;
each R²⁸ is independently hydrogen, substituted or unsubstituted C₁-C₆alkyl, substituted or unsubstituted C₁-C₆fluoroalkyl, —C(═O)R^{b}, or —C(═O)N(R^{b})₂;
each R³⁴ is independently hydrogen or substituted or unsubstituted C₁-C₆alkyl;
each R³⁵ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl, —CN, —OH, —OR^{a}, or —N(R^{b})₂;
m is 0, 1, 2, 3, or 4;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂- or C(═O);
each X³ is independently CR²⁷ or N;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
each R³⁶ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
Ring B is a fused substituted or unsubstituted 5 or 6 membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

49. The compound of claim 45-48, or a pharmaceutically acceptable salt or solvate thereof, wherein the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl; 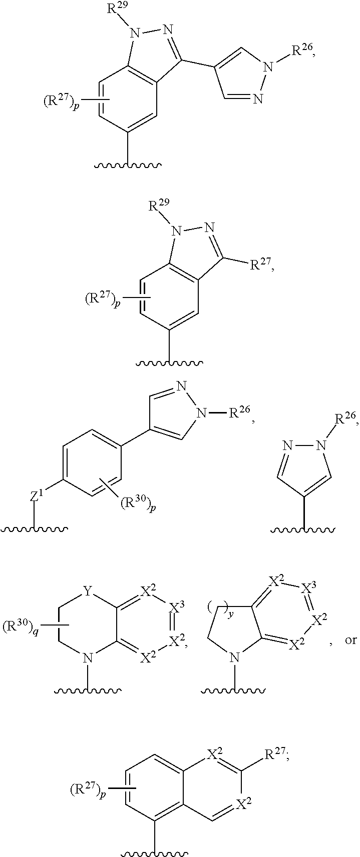
y is 1 or 2;
Z¹ is —NR^{c}—, —O—, or —S—;
Re is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
each X² is independently —CR³⁰— or —N—;
each X³ is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Y is —C(R³⁰)₂- or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
or R³² is -L-C;
L is substituted or unsubstituted C₁-C₆alkyl or substituted or unsubstituted C₁-C₆heteroalkyl;
C is substituted or unsubstituted C₃-C₈ cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

50. The compound of any one of claims 45-49, or a pharmaceutically acceptable salt or solvate thereof, wherein the moiety that occupies the BC Loop region of the bromodomain of CBP/p300 is R³², wherein: 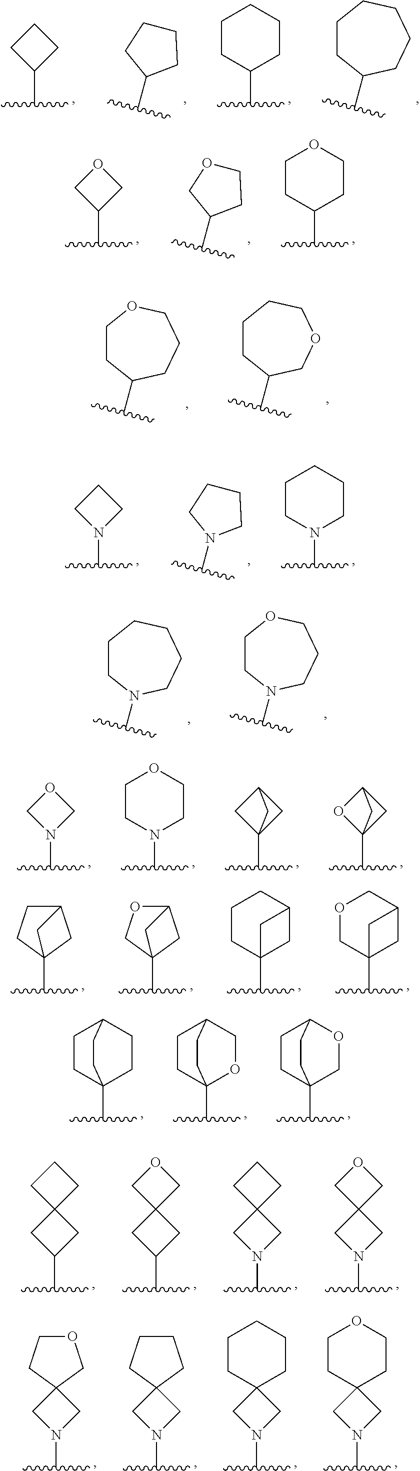 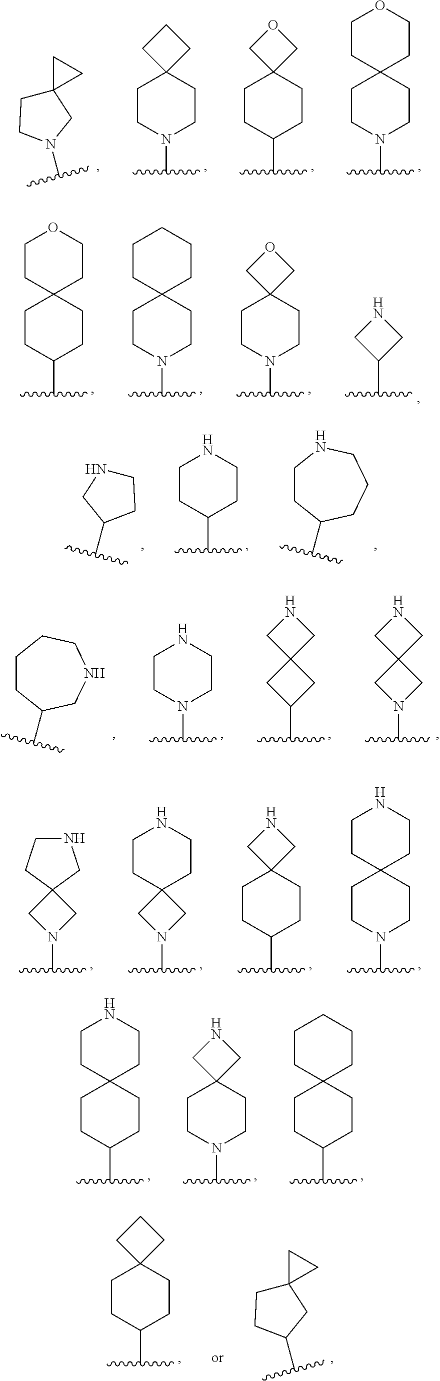
each of which is substituted or unsubstituted.

51. The compound of any one of claims 41-50, or a pharmaceutically acceptable salt or solvate thereof, wherein the moiety comprising an acetyl-lysine mimetic comprises

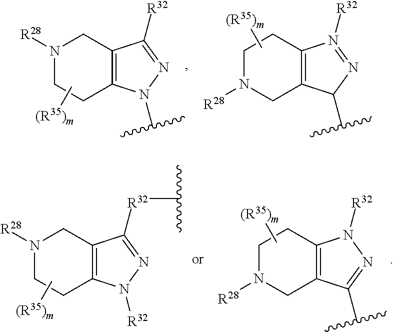

52. The compound of claim 51, or a pharmaceutically acceptable salt or solvate thereof, wherein:
R³² is 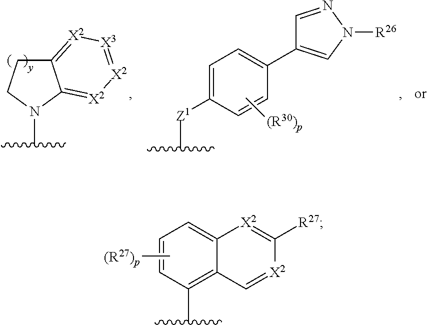
R²⁷ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and
each R²⁷ is independently hydrogen, substituted or unsubstituted C₁-C₆alkyl, substituted or unsubstituted C₁-C₆fluoroalkyl, substituted or unsubstituted C₁-C₆heteroalkyl, —CN, —OH, or —OR^{a}; and
each R^{a} is independently substituted or unsubstituted C₁-C₆alkyl.

53. The compound of claim 52, or a pharmaceutically acceptable salt or solvate thereof, wherein:
each R²⁷ is independently hydrogen, —CH₃, —CH₂CH₃, —F, —CHF₂, —CF₃, —CN, —OH, —OCH₃, cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

54. The compound of any one of claims 51-53, or a pharmaceutically acceptable salt or solvate thereof, wherein the moiety comprising an acetyl-lysine mimetic comprises

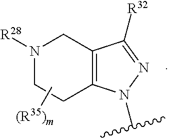

55. The compound of any one of claims 51-53, or a pharmaceutically acceptable salt or solvate thereof, wherein the moiety comprising an acetyl-lysine mimetic comprises

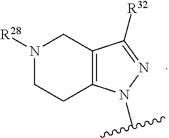

56. The compound of any one of claims 51-55, or a pharmaceutically acceptable salt or solvate thereof, wherein:
R³² is

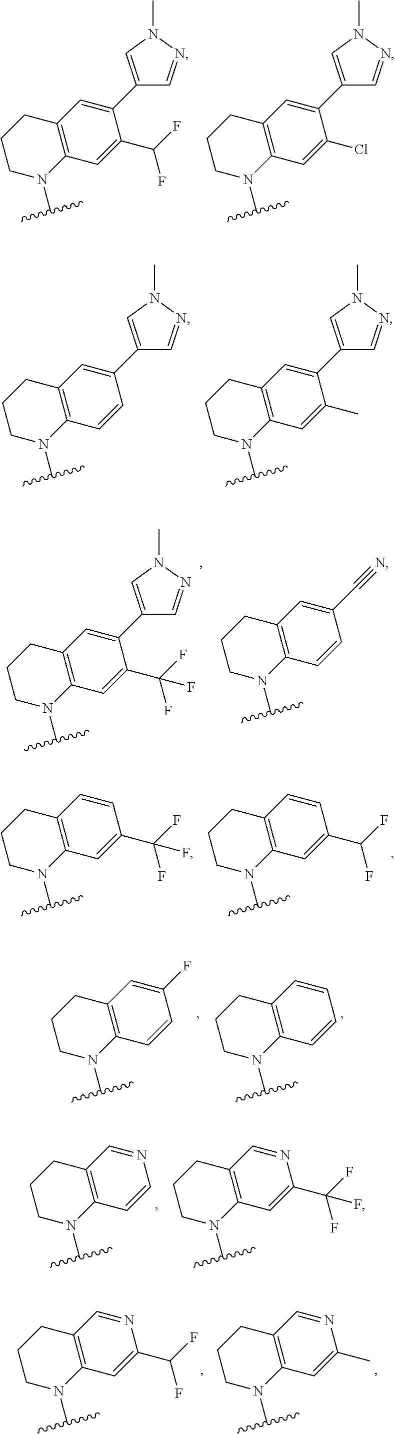

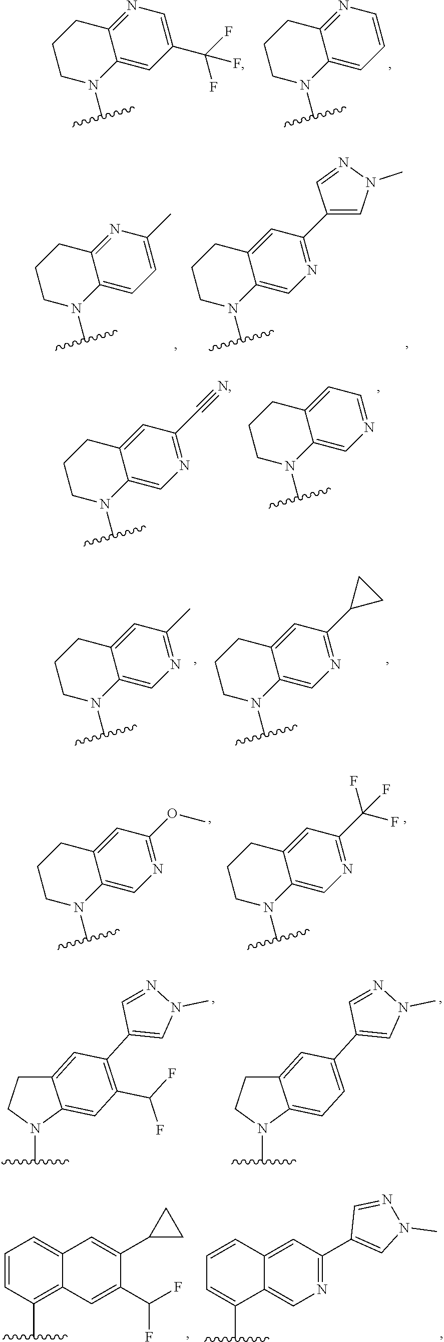

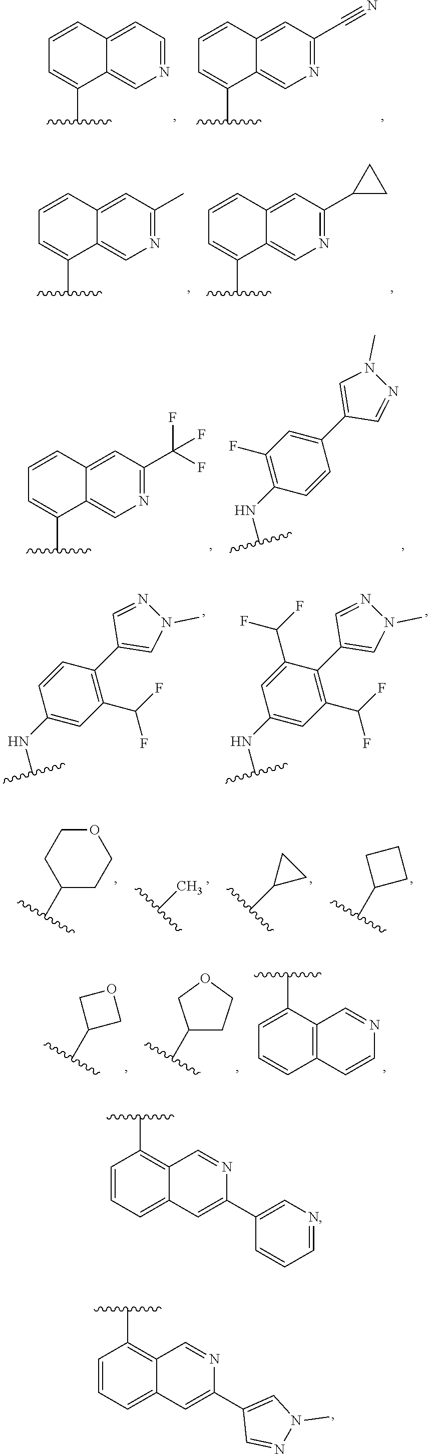

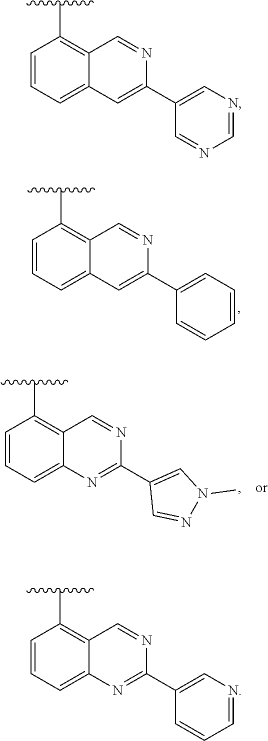 [SMILES: *c1cccc2cc(-c3cncnc3)ncc12 ; *c1cccc2cc(-c3ccccc3)ncc12 ; *c1cccc2nc(-c3cnn(C)c3)ncc12 ; *c1cccc2nc(-c3cccnc3)ncc12]

57. The compound of any one of claims 41-50, or a pharmaceutically acceptable salt or solvate thereof, wherein the moiety comprising an acetyl-lysine mimetic comprises

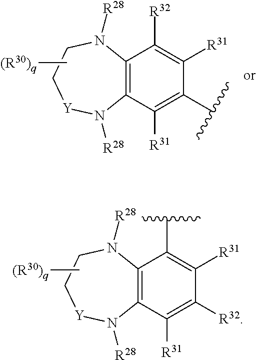

58. The compound of claim 57, or a pharmaceutically acceptable salt or solvate thereof, wherein the moiety comprising an acetyl-lysine mimetic comprises

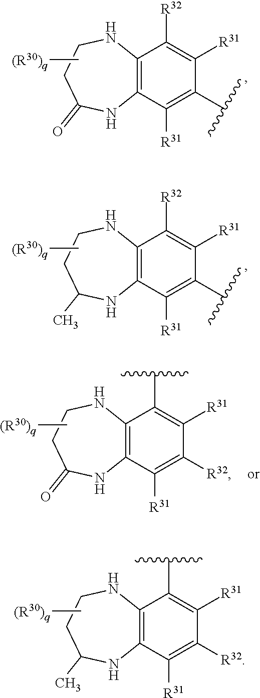

59. The compound of claim 57, or a pharmaceutically acceptable salt or solvate thereof, wherein the moiety comprising an acetyl-lysine mimetic comprises 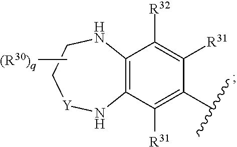
wherein:
R³² is 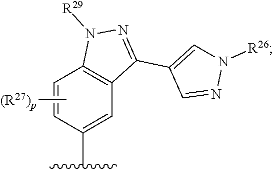
and
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂.

60. The compound of any one of claims 57-59, or a pharmaceutically acceptable salt or solvate thereof, wherein:
R³² is

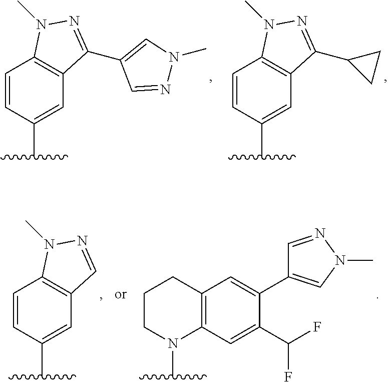

61. The compound of any one of claims 41-50, or a pharmaceutically acceptable salt or solvate thereof, wherein the moiety comprising an acetyl-lysine mimetic comprises

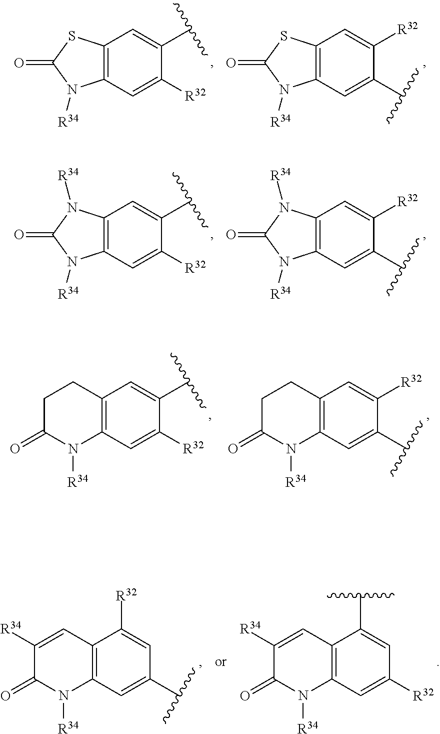

62. The compound of claim 61, or a pharmaceutically acceptable salt or solvate thereof, wherein the moiety comprising an acetyl-lysine mimetic comprises

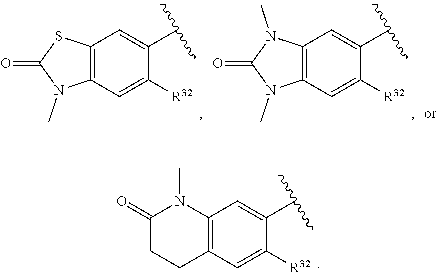

63. The compound of any one of claims 61-62, or a pharmaceutically acceptable salt or solvate thereof, wherein:
R³² is

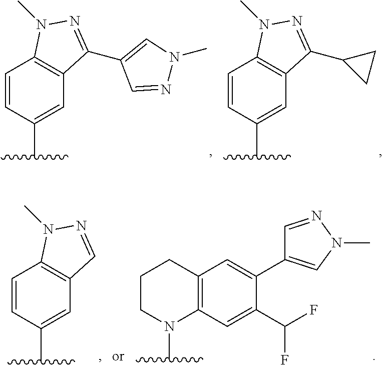

64. The compound of claim 61, or a pharmaceutically acceptable salt or solvate thereof, wherein the moiety comprising an acetyl-lysine mimetic comprises:

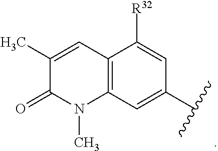

65. The compound of claim 61 or 64, or a pharmaceutically acceptable salt or solvate thereof, wherein:
R³² is

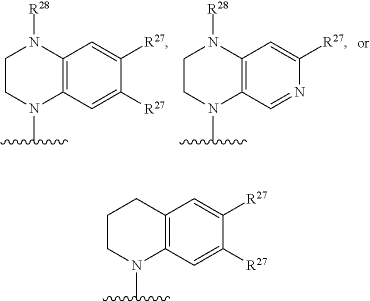

66. The compound of any one of claims 61, 62, 64, or 65, or a pharmaceutically acceptable salt or solvate thereof, wherein:
R³² is

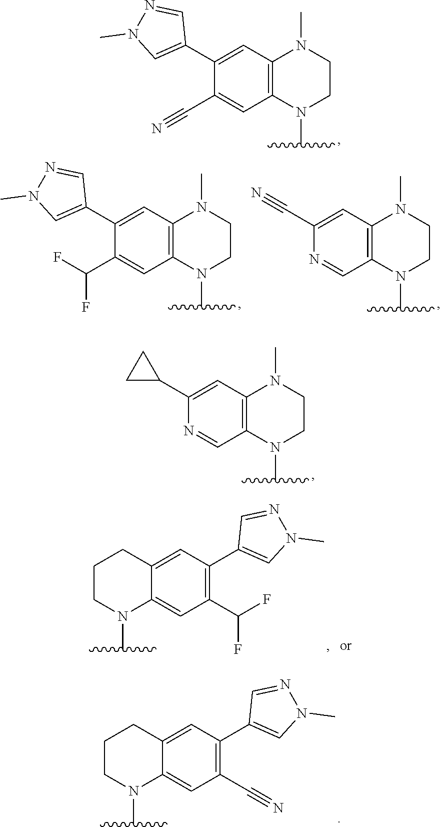

67. The compound of any one of claims 41-50, or a pharmaceutically acceptable salt or solvate thereof, wherein the moiety comprising an acetyl-lysine mimetic comprises

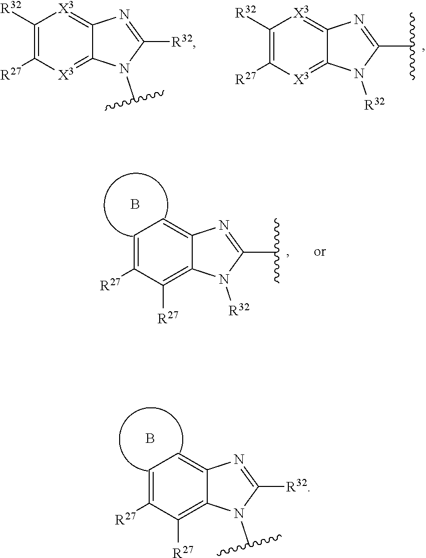

68. The compound of claim 67, or a pharmaceutically acceptable salt or solvate thereof, wherein the moiety comprising an acetyl-lysine mimetic comprises 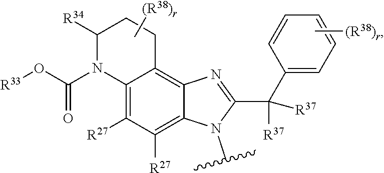
wherein:
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
R³³ is hydrogen, substituted or unsubstituted C₁-C₆alkyl, substituted or unsubstituted C₁-C₆fluoroalkyl, or substituted or unsubstituted C₁-C₆heteroalkyl;
R³⁴ is hydrogen or substituted or unsubstituted C₁-C₆alkyl;
each R³⁸ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, —CN, —NO₂, —OH, —OR^{a}, —OC(═O)R^{a}, —OC(═O)N(R^{b})₂, —OC(═O)OR^{a}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
r is 0, 1, 2, 3, or 4;

69. The compound of claim 67, or a pharmaceutically acceptable salt or solvate thereof, wherein:

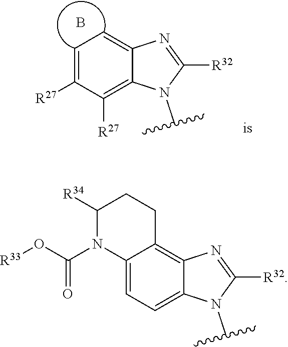

70. The compound of claim 67, or a pharmaceutically acceptable salt or solvate thereof, wherein the moiety comprising an acetyl-lysine mimetic comprises

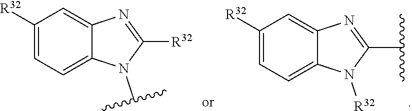

71. The compound of any one of claims 67-70, or a pharmaceutically acceptable salt or solvate thereof, wherein:
R³² is

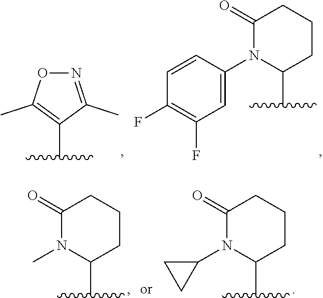

72. The compound of any one of claims 67-70, or a pharmaceutically acceptable salt or solvate thereof, wherein:

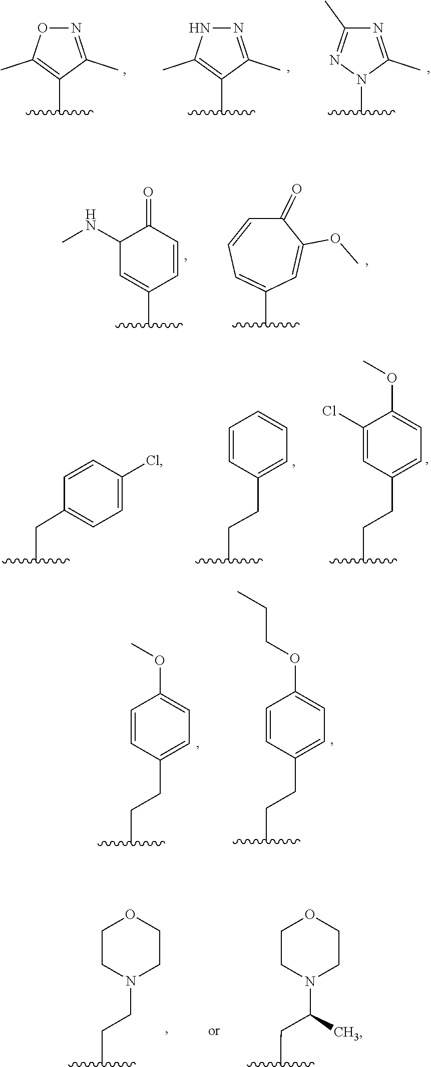

73. A heterobifunctional conditional inhibitor compound of Formula (I) or a pharmaceutically acceptable salt or solvate thereof: 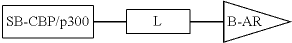
wherein:
SB-CBP/p300 is a silent binder of the bromodomain of human CREB-binding protein (CBP) or human E1A-binding protein p300 (p300) (CBP/p300), wherein the binder of CBP/p300 comprises:
an acetyl-lysine mimetic moiety that binds in the acetyl-lysine (KAc) binding site of the bromodomain of human CREB-binding protein (CBP) or human E1A-binding protein p300 (p300) (CBP/p300) and has the structure: 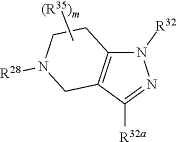
R²⁸ is —C(═O)CH₃, —C(═O)CH₂CH₃, —C(═O)NH₂, —C(═O)NH(CH₃); or —C(═O)NH(CH₂CH₃);
R³² is a moiety that occupies the BC Loop region of the bromodomain of CBP/p300;
R^{32a} is a moiety that occupies the lipophilic shelf (LPF) region of the bromodomain of CBP/p300;
each R³⁵ is independently hydrogen, C₁-C₄alkyl, or C₁-C₄fluoroalkyl;
m is 0, 1, 2, 3, or 4;
L is an optional linker;
wherein L is covalently attached to the R³² group, or at the position occupied by R³², or the R^{32a} group;
B-AR is a binder of the androgen receptor (AR), wherein the B-AR comprises:
a head group that occupies the ligand-binding domain (LBD) of AR and is covalently attached to a core with a tail moiety covalently attached to the core moiety; wherein B-AR has the structure of Formula (D): 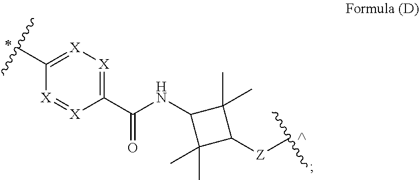
wherein head group is 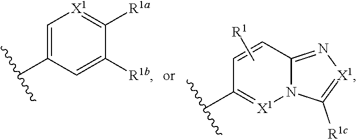
and the the head group is covalently attached to Z at position (\hat{ }), and L is attached at position *;
Z is —O—, —NH— or —N(C₁-C₄alkyl)-;
each X¹ is independently —CR¹— or —N—;
R^{1a} is —CN, —NO₂, —C(═O)R⁵, —C(═O)OR⁵, or —C(═O)N(R⁵)₂;
R^{1b} is hydrogen, halogen, substituted or unsubstituted C₁-C₄alkyl, C₁-C₄fluoroalkyl, —CN, —OH, or —OR⁴;
R^{1c} is hydrogen, halogen, C₁-C₄alkyl, C₁-C₄fluoroalkyl, or —CN;
each R¹ is independently hydrogen, halogen, substituted or unsubstituted C₁-C₄alkyl, C₁-C₄fluoroalkyl, —CN, —OH, or —OR⁴;
each X is independently —CR³— or —N—;
each R³ is independently hydrogen, halogen, substituted or unsubstituted C₁-C₄alkyl, C₁-C₄fluoroalkyl, —CN, —OH, —OR⁴, or —N(R⁵)₂.
each R⁴ is independently substituted or unsubstituted C₁-C₄alkyl, or C₁-C₄fluoroalkyl;
each R⁵ is independently hydrogen, substituted or unsubstituted C₁-C₄alkyl, C₁-C₄fluoroalkyl; and
each s is 1, 2, or 3.

74. The compound of claim 73, or a pharmaceutically acceptable salt or solvate thereof, wherein:
R³² is hydrogen, substituted or unsubstituted C₁-C₆alkyl, substituted or unsubstituted C₃-C₁₂cycloalkyl, or substituted or unsubstituted 3- to 12-membered heterocycloalkyl;
R^{32a} is 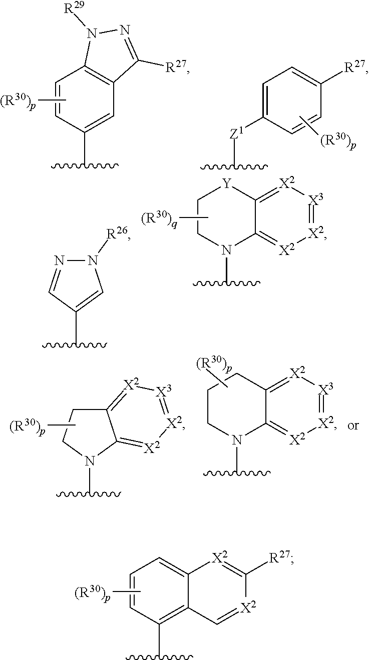
at least one X² is —CR³⁰— and at most two X² are —N—;
Z¹ is —NR^{c}— or —O—;
R^{c} is hydrogen or C₁-C₆alkyl;
R²⁶ is hydrogen, substituted or unsubstituted C₁-C₄alkyl, C₁-C₄fluoroalkyl, substituted or unsubstituted C₃-C₈ cycloalkyl, or substituted or unsubstituted 3- to 8-membered heterocycloalkyl;
X³ is —CR²⁷— or —N—;
R²⁷ is hydrogen, halogen, substituted or unsubstituted C₁-C₄alkyl, C₁-C₄fluoroalkyl, substituted or unsubstituted C₃-C₈ cycloalkyl, substituted or unsubstituted 3- to 8-membered heterocycloalkyl, substituted or unsubstituted phenyl, substituted or unsubstituted monocyclic heteroaryl, —CN, —OH, —OR^{a}, —N(R^{b})₂, —NR^{b}C(═O)R^{a}, —C(═O)R^{b}, —C(═O)OR^{b}, or —C(═O)N(R^{b})₂;
or R²⁷ is 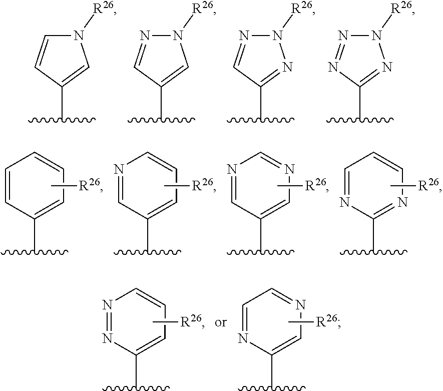
p is 0, 1, 2, or 3;
R²⁹ is hydrogen, substituted or unsubstituted C₁-C₄alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Y is —C(R³⁰)₂- or N(R²⁸)—;
each R³⁰ is independently hydrogen, halogen, C₁-C₄alkyl, C₁-C₄fluoroalkyl, substituted or unsubstituted C₁-C₆heteroalkyl, —CN, —OH, or —OR^{a};
q is 0, 1, 2, 3, or 4;
each R^{a} is independently C₁-C₄alkyl, C₁-C₆fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
each R^{b} is independently hydrogen, C₁-C₄alkyl, C₁-C₄fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
or two R^{b} on the same N atom are taken together with the N atom to which they are attached to form a substituted or unsubstituted N-containing heterocycle.

75. The compound of any one of claims 73-74, or a pharmaceutically acceptable salt or solvate thereof, wherein:
R²⁷ is hydrogen, halogen, C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic 5- or 6-membered heteroaryl, —CN, —OH, —OR^{a}, —N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
or R²⁷ is

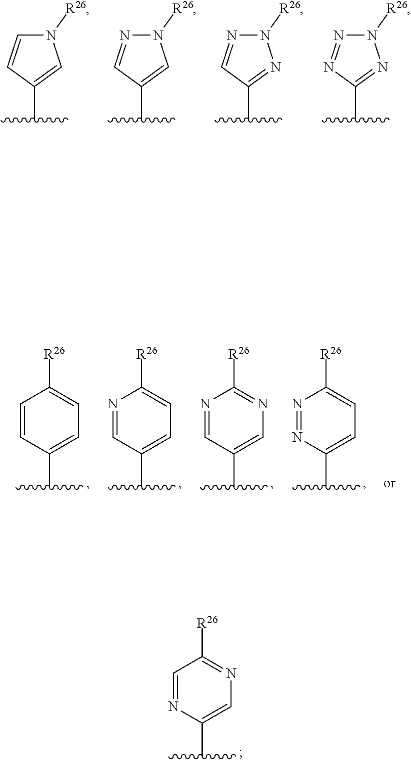

76. The compound of any one of claims 73-75, or a pharmaceutically acceptable salt or solvate thereof, wherein:
R³² is 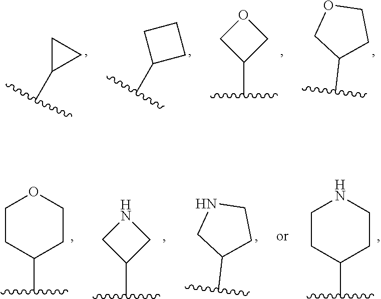
each of which is unsubstituted or substituted with F, Cl, Br, —CH₃, —CD₃, —CH₂CH₃, —CH₂F, —CHF₂, —CF₃, —CH₂CH₂F, —CH₂CHF₂, —CH₂CF₃, —CN, —C(═O)CH₃, —C(═O)CH₂CH₃, —C(═O)CH₂F, —C(═O)CHF₂, —C(═O)CF₃, —C(═O)CH₂CH₂F, —C(═O)CH₂CHF₂, —C(═O)CH₂CF₃, —C(═O)CD₃, or —SO₂CH₃, —SO₂CH₂CH₃, —SO₂CD₃, or —SO₂CH₂CD₃.
R^{32a} is 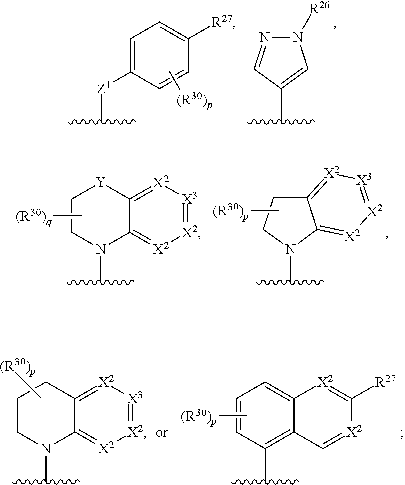
at least one X² is —CR³⁰— and at most two X² are —N—;
each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted aryl, substituted or unsubstituted heteroaryl, —CN, —OH, or —OR^{a}; and
each R^{a} is independently substituted or unsubstituted C₁-C₆alkyl.

77. The compound of any one of claims 73-76, or a pharmaceutically acceptable salt or solvate thereof, wherein:
R²⁷ is hydrogen, —CH₃, —CH₂CH₃, —CH₂CH₂CH₃, —CH₂(CH₃)₂, —(CH₃)₃, —F, —CHF₂, —CF₃, —CN, —OH, —OCH₃, cyclopropyl, cyclobutyl, oxetanyl, tetrahydrofuranyl, tetrahydropyranyl, unsubstituted or substituted phenyl, unsubstituted or substituted pyrazolyl, unsubstituted or substituted pyridinyl, or unsubstituted or substituted pyrimidinyl; or
R²⁷ is 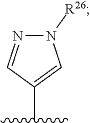
R³² is 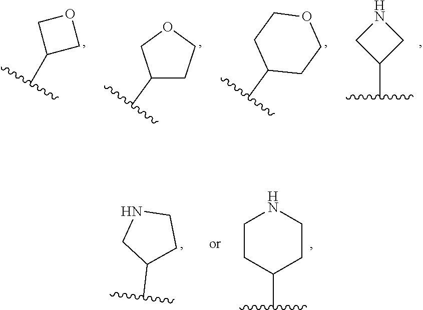
each of which is unsubstituted or substituted with F, Cl, Br, —CH₃, —CD₃, —CH₂CH₃, —CH₂F, —CHF₂, —CF₃, CN, —C(═O)CH₃, —C(═O)CH₂F, —C(═O)CHF₂, —C(═O)CF₃, —C(═O)CD₃.

78. The compound of claim 73-77, or a pharmaceutically acceptable salt or solvate thereof, wherein:
R³² is 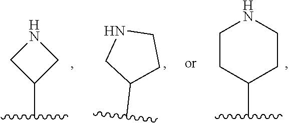 [SMILES: *C1CNC1 ; *C1CCNC1 ; *C1CCNCC1]
wherein the optional linker is covalently attached to the nitrogen of R³² group;
or R³² is absent and L is covalently attached to the acetyl-lysine mimetic moiety at the position occupied by R³²; and
R^{32a} is

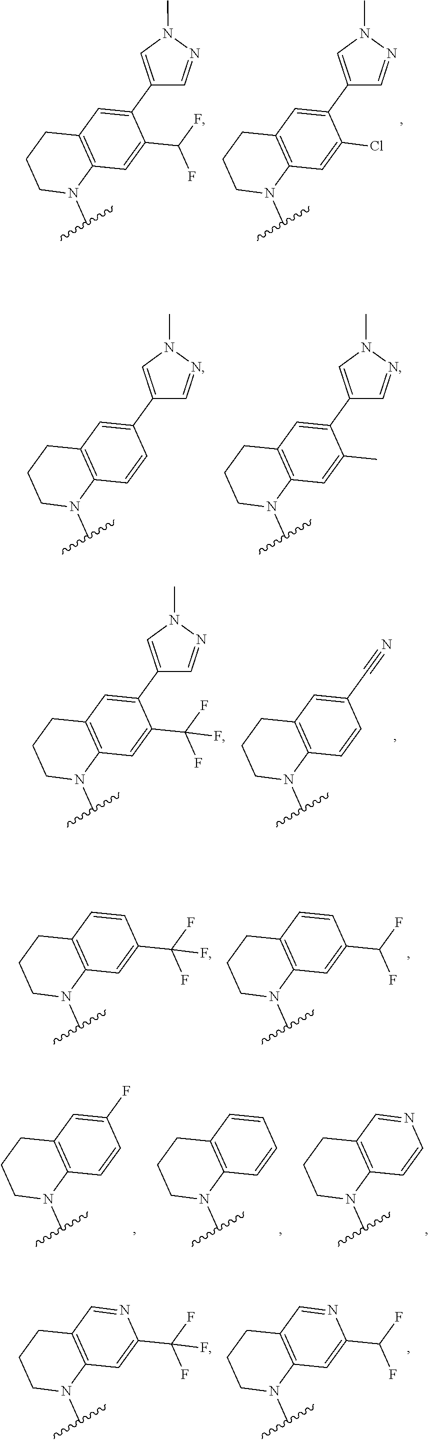

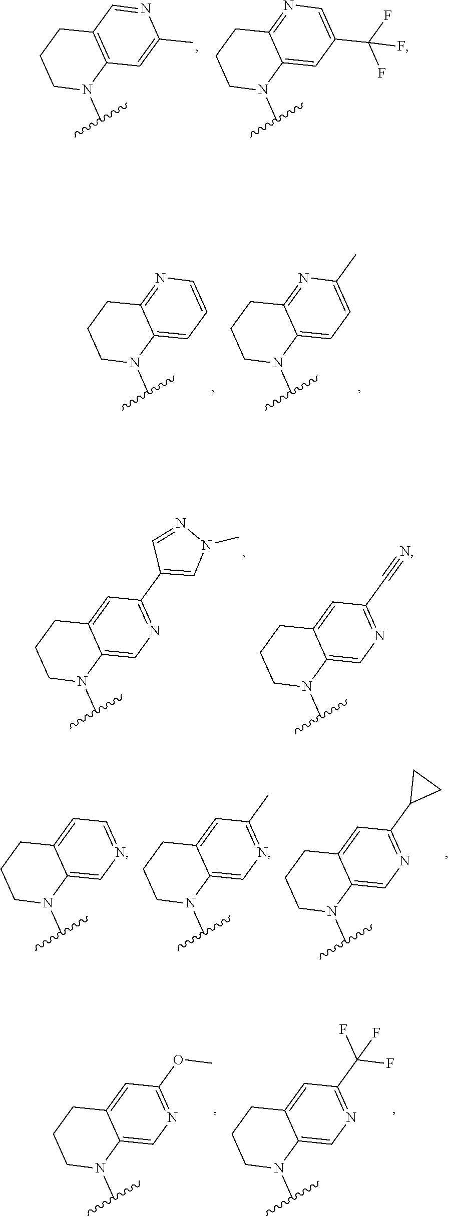

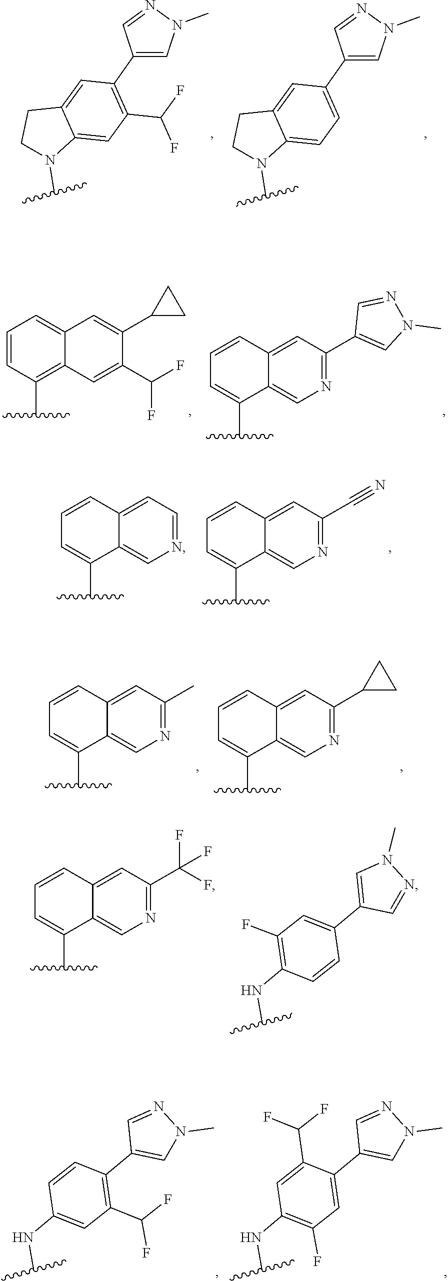

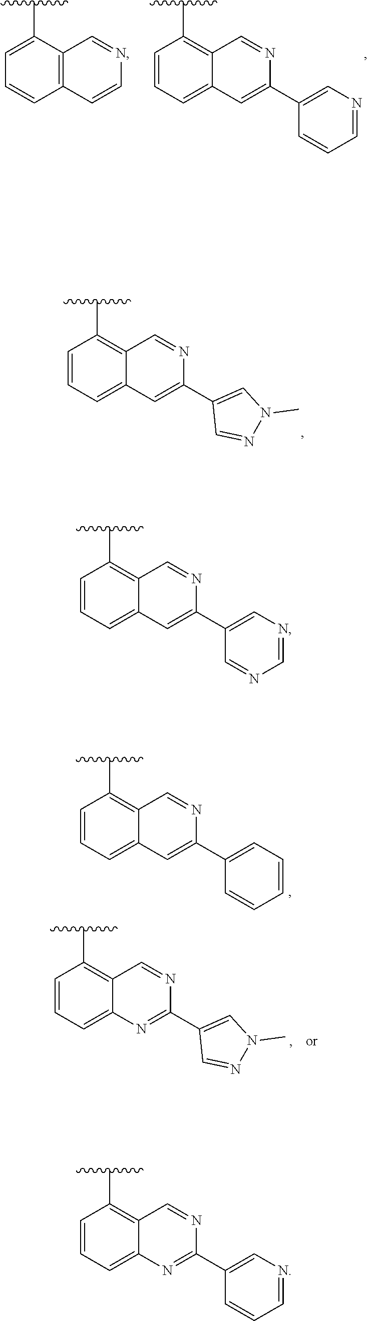

79. The compound of any one of claims 73-78, or a pharmaceutically acceptable salt or solvate thereof, wherein:
R³² is 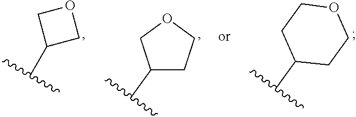 [SMILES: *C1COC1 ; *C1CCOC1 ; *C1CCOCC1]
R^{32a} is 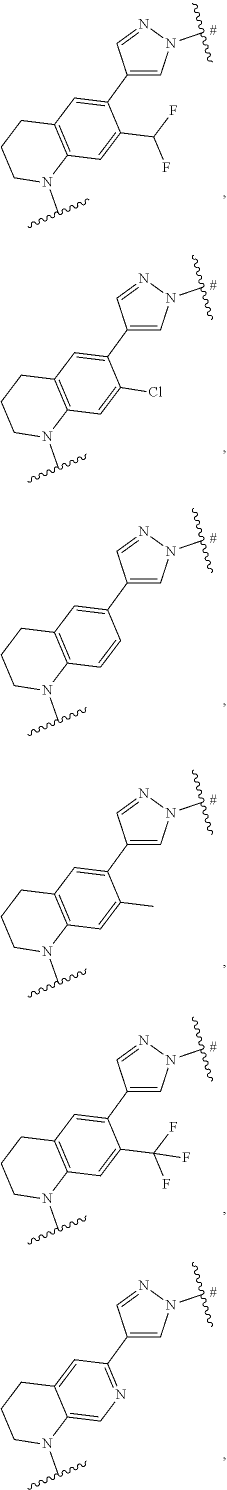 [SMILES: *N1CCCc2cc(-c3cnn(*)c3)c(C(F)F)cc21 ; *N1CCCc2cc(-c3cnn(*)c3)c(Cl)cc21 ; *N1CCCc2cc(-c3cnn(*)c3)ccc21 ; *N1CCCc2cc(-c3cnn(*)c3)c(C)cc21 ; *N1CCCc2cc(-c3cnn(*)c3)c(C(F)(F)F)cc21 ; *N1CCCc2cc(-c3cnn(*)c3)ncc21] 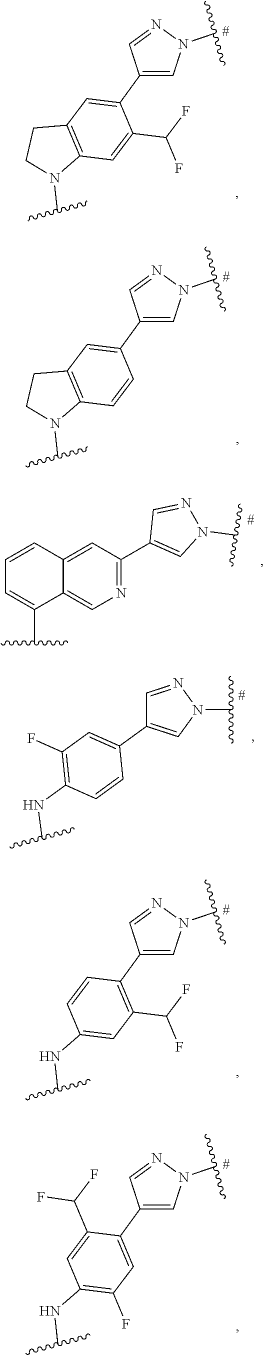 [SMILES: *N1CCc2cc(-c3cnn(*)c3)c(C(F)F)cc21 ; *N1CCc2cc(-c3cnn(*)c3)ccc21 ; *c1cccc2cc(-c3cnn(*)c3)ncc12 ; *Nc1ccc(-c2cnn(*)c2)cc1F ; *Nc1ccc(-c2cnn(*)c2)c(C(F)F)c1 ; *Nc1cc(C(F)F)c(-c2cnn(*)c2)cc1F] 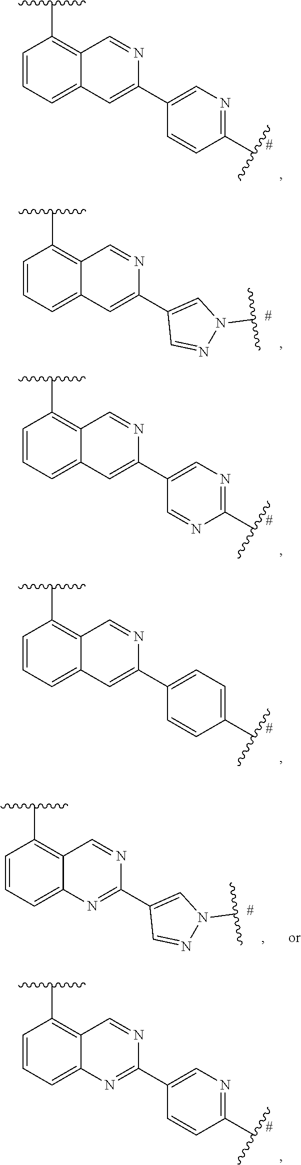 [SMILES: *c1ccc(-c2cc3cccc(*)c3cn2)cn1 ; *c1cccc2cc(-c3cnn(*)c3)ncc12 ; *c1ncc(-c2cc3cccc(*)c3cn2)cn1 ; *c1ccc(-c2cc3cccc(*)c3cn2)cc1 ; *c1cccc2nc(-c3cnn(*)c3)ncc12 ; *c1ccc(-c2ncc3c(*)cccc3n2)cn1]
wherein 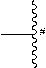
is the point of attachment to the optional linker.

80. The compound of claim 73, or a pharmaceutically acceptable salt or solvate thereof, wherein the head group is: 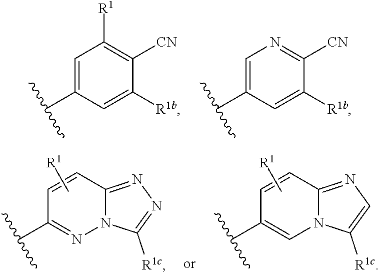
R^{1b} is hydrogen, F, Cl, Br, I, —CH₃, —CD₃, —CH₂CH₃, —CH₂F, —CHF₂, —CF₃, —OH, —OCF₃, —OCH₃, —OCH₂CH₃, —OCD₃, —OCH₂CD₃, or —CN;
each R^{1c} is hydrogen, F, Cl, Br, —CH₃, —CD₃, —CH₂F, —CHF₂, or —CF₃;
each R¹ is independently hydrogen, F, Cl, Br, I, —CH₃, —CD₃, —CH₂CH₃, —CH₂F, —CHF₂, —CF₃, —CH₂CH₂F, —CH₂CHF₂, —CH₂CF₃, —OH, —OCF₃, —OCH₃, —OCD₃, or —OCH₂CH₃.

81. The compound of claim 73, or a pharmaceutically acceptable salt or solvate thereof, wherein the head group is selected from:

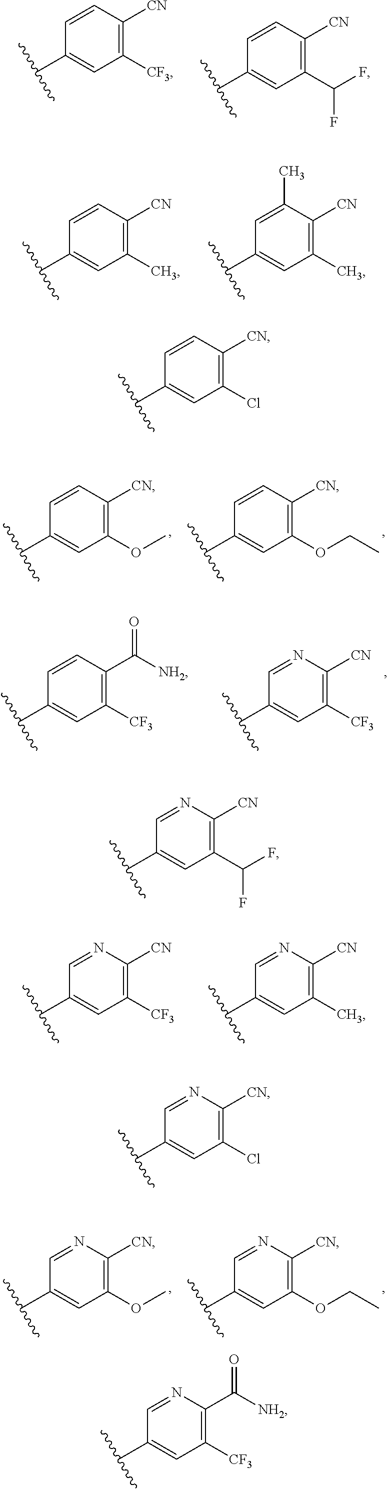

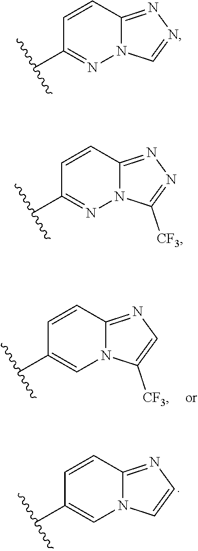 [SMILES: *c1ccc2nncn2n1 ; *c1ccc2nnc(C(F)(F)F)n2n1 ; *c1ccc2ncc(C(F)(F)F)n2c1 ; *c1ccc2nccn2c1]

82. The compound of any one of claims 14-81, or a pharmaceutically acceptable salt or solvate thereof, wherein:
SB-CBP/p300 has the structure: 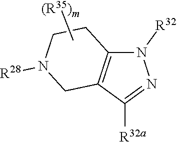
R²⁸ is —C(═O)CH₃ or —C(═O)NH(CH₃);
each R³⁵ is independently hydrogen, —CH₃, —CH₂F, —CHF₂, —CF₃;
m is 0, 1, or 2;
R³² is 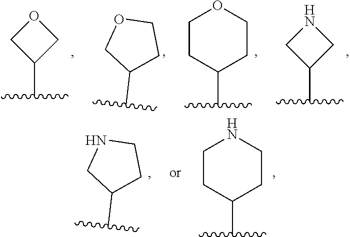
each of which is unsubstituted or substituted with F, —CH₃, —CH₂F, —CHF₂, or —CF₃;
or R³² is absent and the optional linker is covalently attached to the acetyl-lysine mimetic moiety at the position occupied by R³²;
R^{32a} is 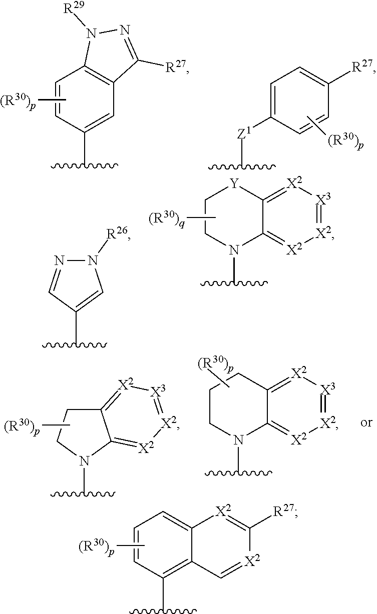
at least one X² is —CR³⁰— and at most two X² are —N—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is 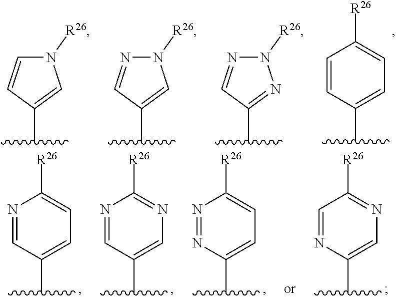
each R³⁰ is independently hydrogen, halogen, C₁-C₄alkyl, C₁-C₄fluoroalkyl, —CN, —OH, or —OR^{a};
R^{a} is C₁-C₄alkyl;
each R^{b} is independently hydrogen or C₁-C₆alkyl;
wherein L is covalently attached to the R³² group, or at the position occupied by R²⁶, or at the position occupied by R²⁷;
B-AR has the structure: 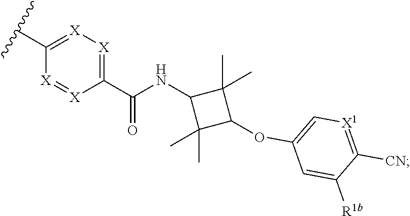
each X is independently —CR³— or —N—;
X¹ is —CR¹— or —N—;
R^{1b} is hydrogen, F, Cl, —CH₃, —CH₂F, —CHF₂, —CF₃, or —OCF₃;
R¹ is hydrogen, F, Cl, —CH₃, —CH₂F, —CHF₂, or —CF₃; 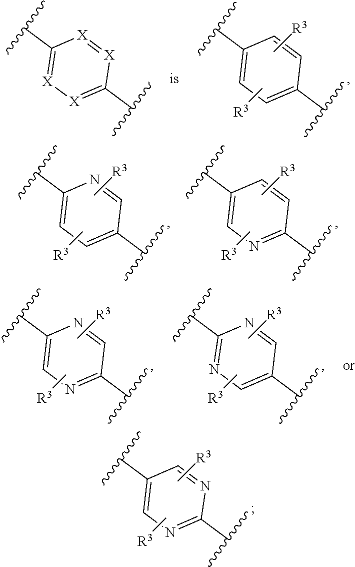
each R³ is independently hydrogen, F, Cl, Br, —CH₃, —CH₂F, —CHF₂, —CF₃, —OH, —OCF₃, —OCH₃, or —CN.

83. The compound of claim 82, or a pharmaceutically acceptable salt or solvate thereof, wherein:

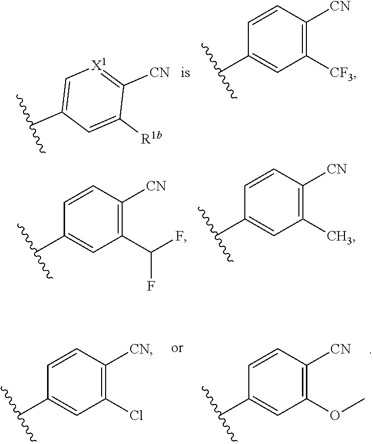

84. The compound of any one of claims 82-83, or a pharmaceutically acceptable salt or solvate thereof, wherein:
R³² is 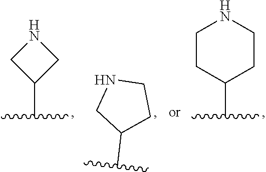 [SMILES: *C1CNC1 ; *C1CCNC1 ; *C1CCNCC1]
wherein the optional linker is covalently attached to the nitrogen of R³² group;
or R³² is absent and the optional linker is covalently attached to the acetyl-lysine mimetic moiety at the position occupied by R³²;
R^{32a} is 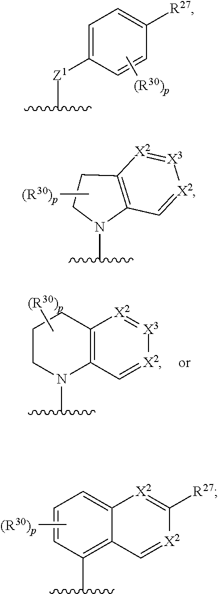
at least one X² is —CR³⁰— and at most two X² are —N—;
X³ is —CR²⁷— or —N—;
Z¹ is —NH— or —O—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is 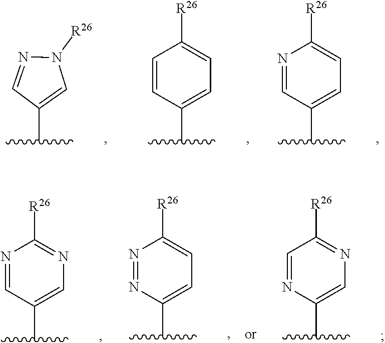
and
each R³⁰ is independently hydrogen, halogen, C₁-C₄alkyl, C₁-C₄fluoroalkyl, —CN, —OH, or —OR^{a}.

85. The compound of any one of claims 82-84, or a pharmaceutically acceptable salt or solvate thereof, wherein:
R³² is 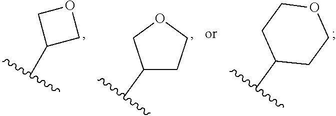 [SMILES: *C1COC1 ; *C1CCOC1 ; *C1CCOCC1]
R^{32a} is 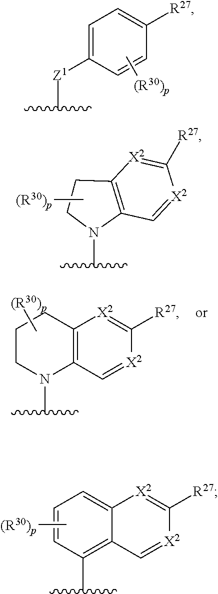
R²⁷ is 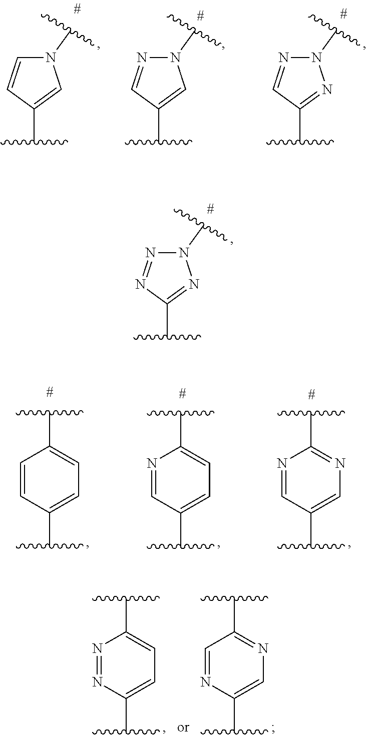
Z¹ is —NH— or —O—; 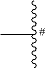
is the point of attachment to the optional linker.

86. The compound of any one of claims 14-81, or a pharmaceutically acceptable salt or solvate thereof, wherein SB-CBP/p300 has the structure of Formula (IIIb): 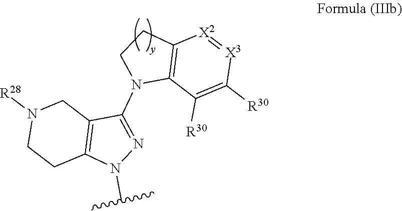
wherein:
X² is —CR³⁰— or —N—;
X³ is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
each R³⁰ is independently hydrogen, halogen, C₁-C₄alkyl, C₁-C₄fluoroalkyl, —CN, —OH, or —OR^{a};
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

87. The compound of any one of claims 14-81, or a pharmaceutically acceptable salt or solvate thereof, wherein SB-CBP/p300 has the structure of Formula (IIIc): 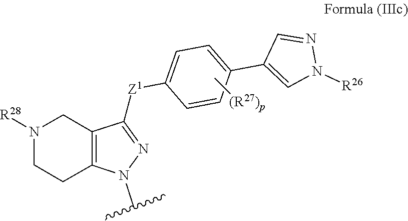
wherein:
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z¹ is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

88. The compound of any one of claims 14-81, or a pharmaceutically acceptable salt or solvate thereof, wherein SB-CBP/p300 has the structure of Formula (IIId-1) or (IIId-2): 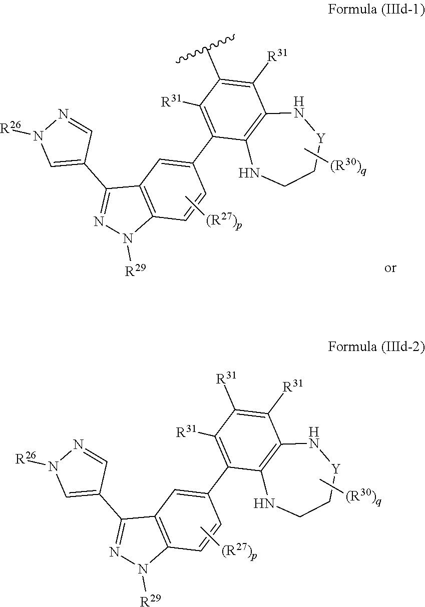
wherein:
R²⁶ is hydrogen, substituted or unsubstituted C₁-C₆alkyl, substituted or unsubstituted C₁-C₆heteroalkyl, substituted or unsubstituted C₃-C₈ cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂- or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

89. The compound of any one of claims 14-81, or a pharmaceutically acceptable salt or solvate thereof, wherein SB-CBP/p300 has the structure of Formula (IIIe): 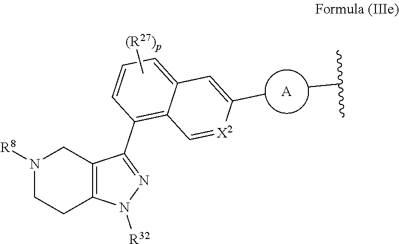
wherein:
X² is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, or substituted or unsubstituted 3- to 8-membered heterocycloalkyl;
Ring A is absent or substituted or unsubstituted C₃-C₈ cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

90. The compound of any one of claims 14-89, or a pharmaceutically acceptable salt or solvate thereof, wherein SB-CBP/p300 has one of the following structures:

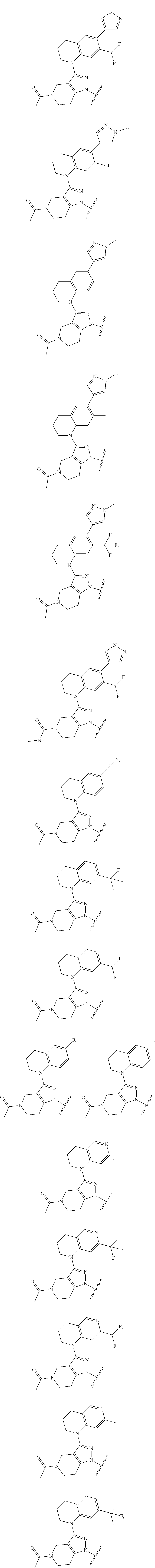

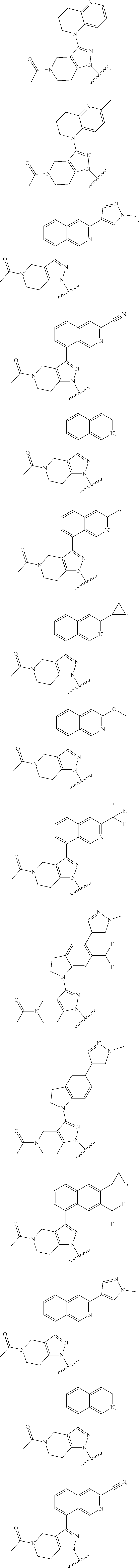

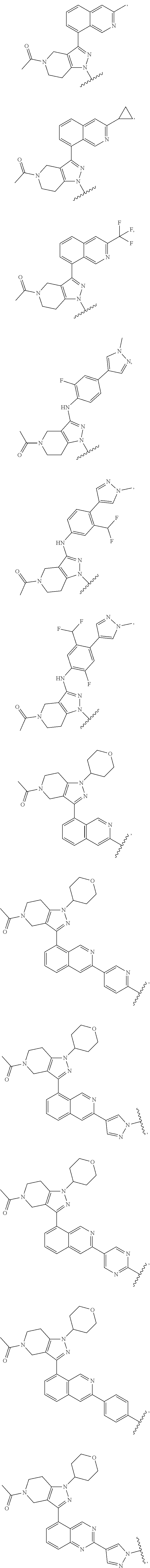

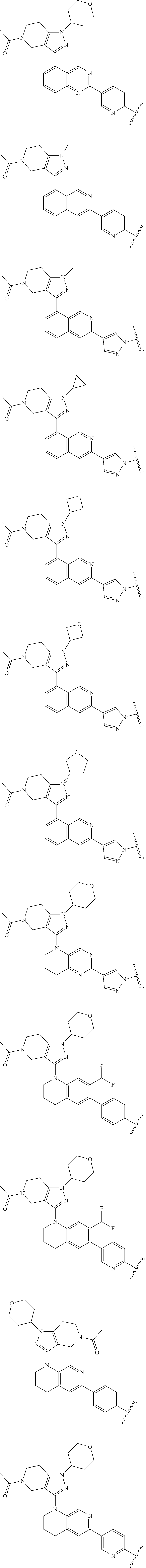

[chemical structure]

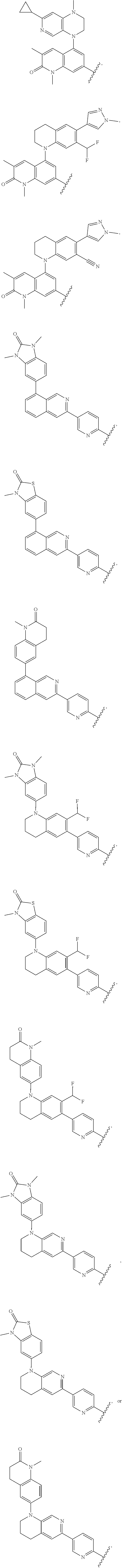

91. The compound of any one of claims 14-89, wherein SB-CBP/p300 has one of the following structures: 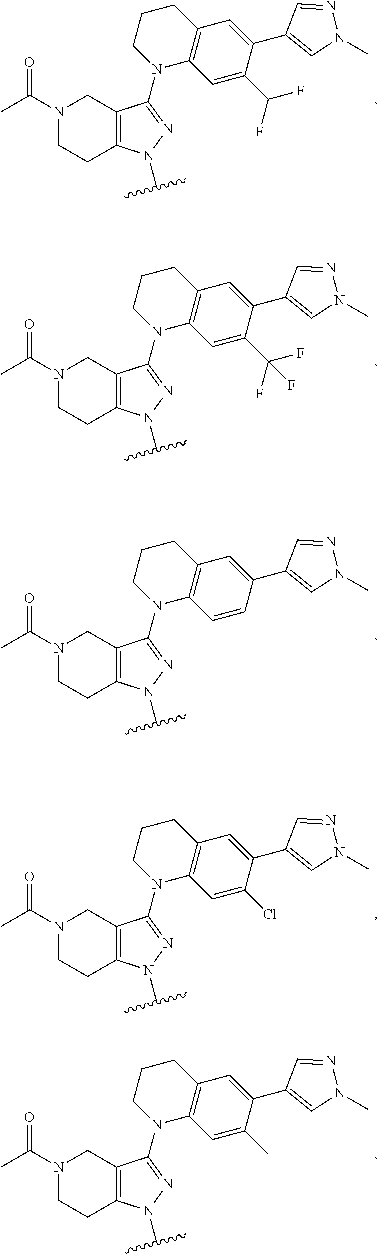 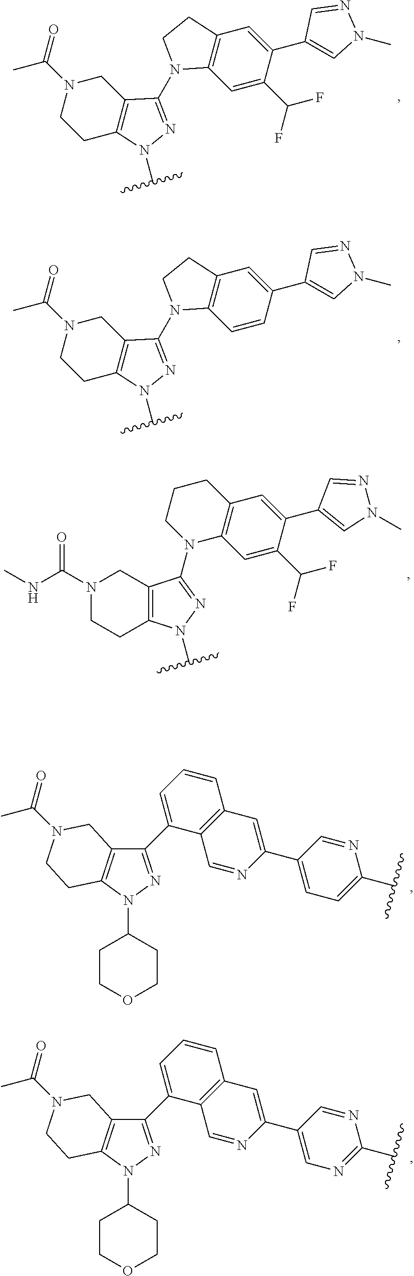 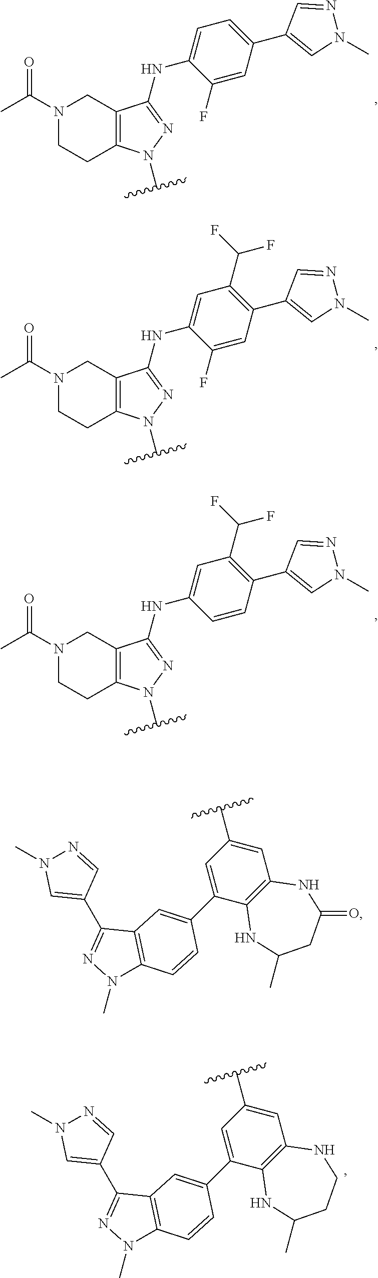 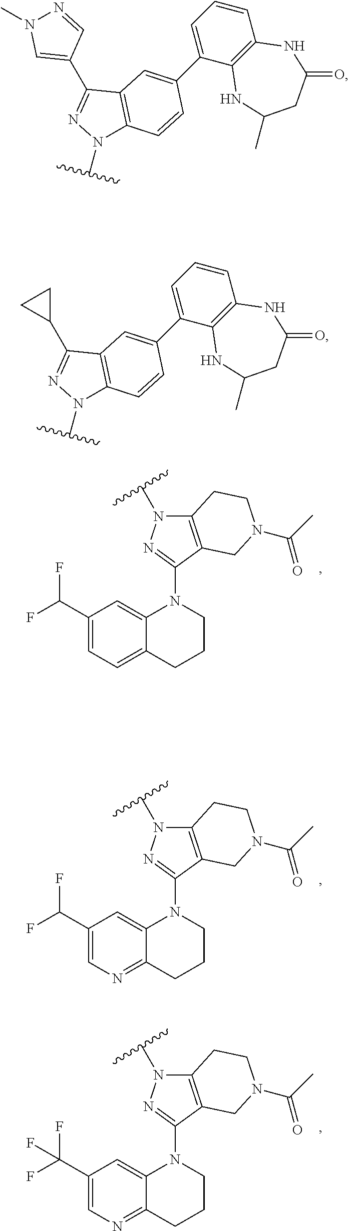 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2] 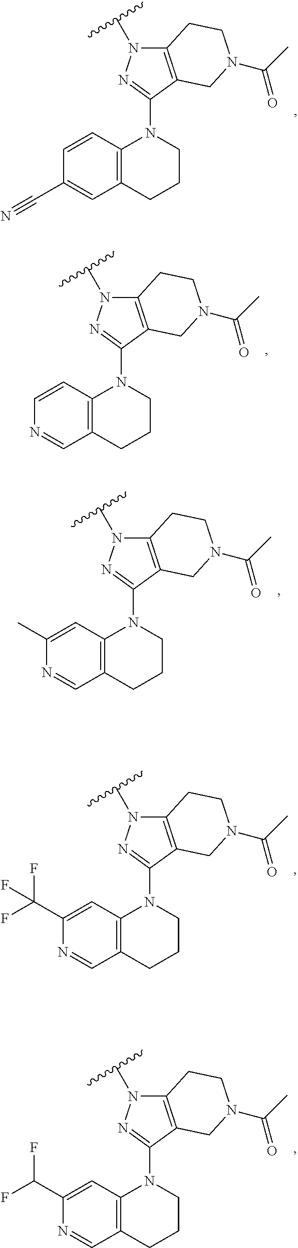 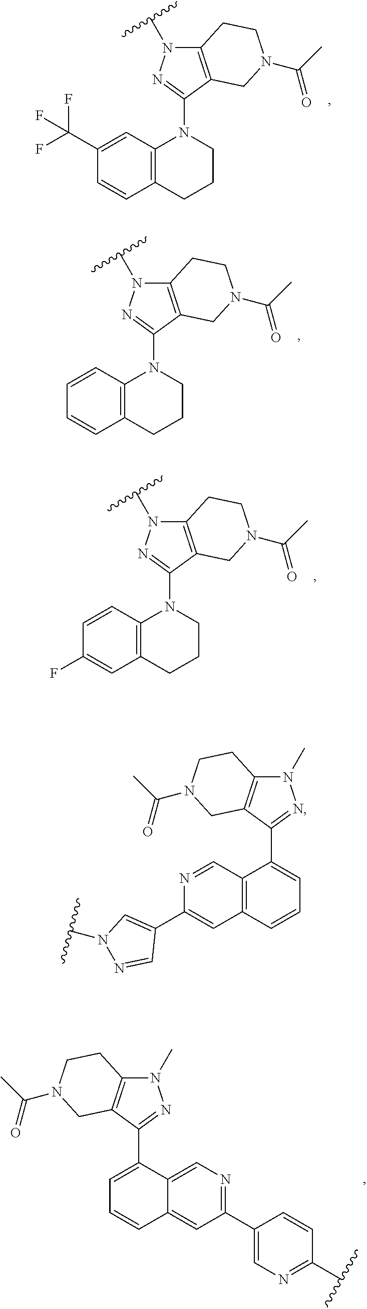 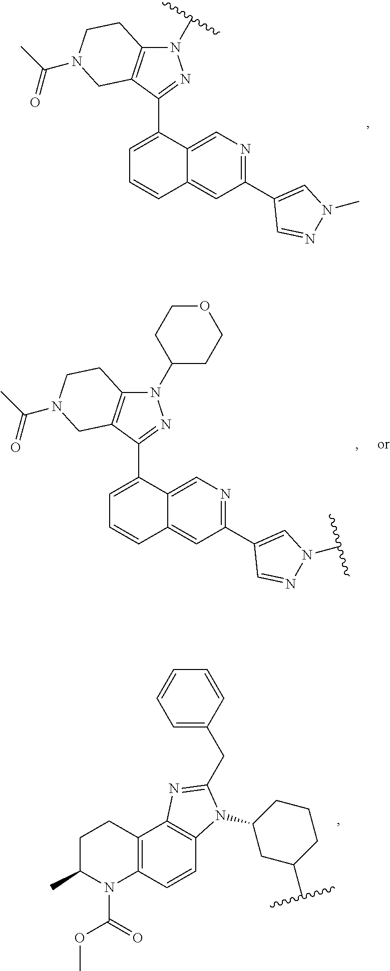
or a pharmaceutically acceptable salt or solvate thereof.

92. The compound of any one of claims 14-89, or a pharmaceutically acceptable salt or solvate thereof, wherein SB-CBP/p300 has one of the following structures:

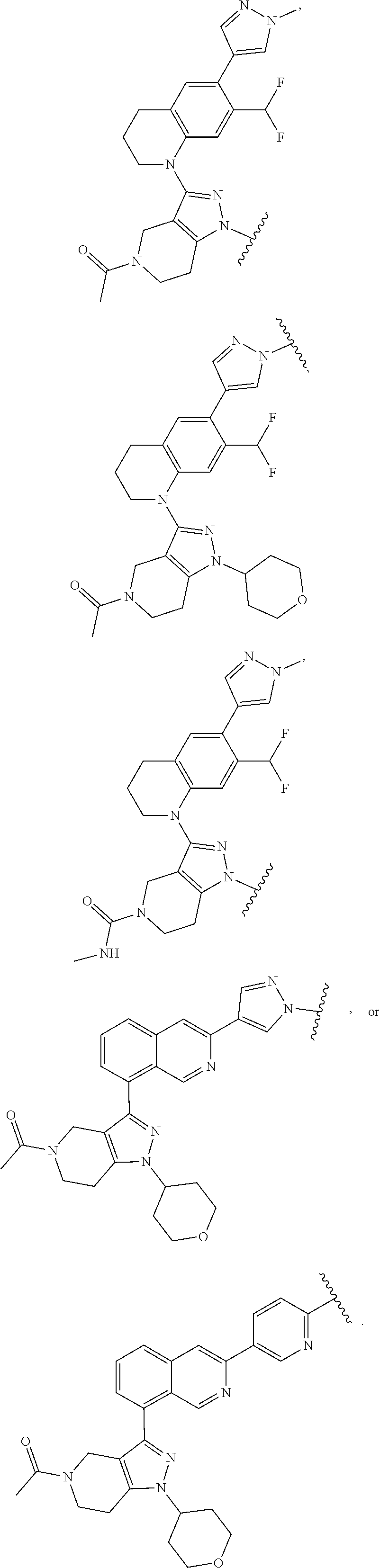

93. The compound of any one of claims 14-89, or a pharmaceutically acceptable salt or solvate thereof, wherein SB-CBP/p300 has one of the following structures:

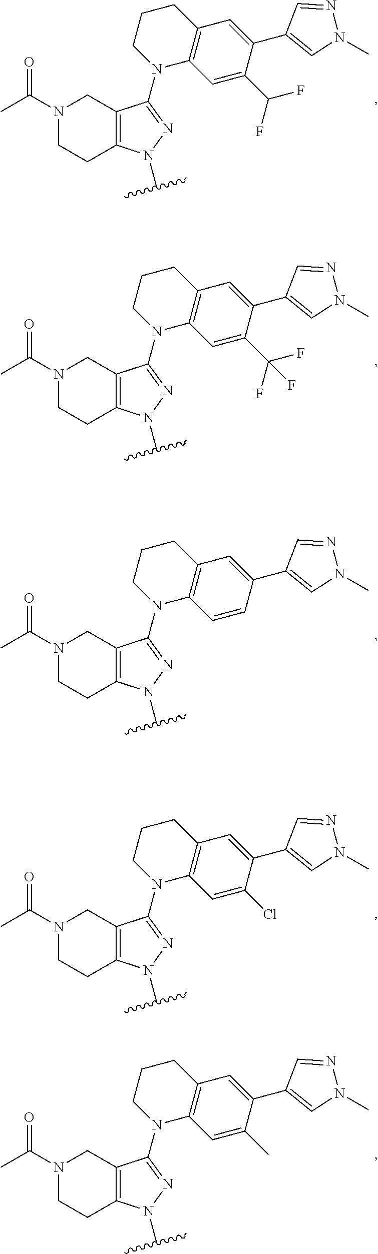

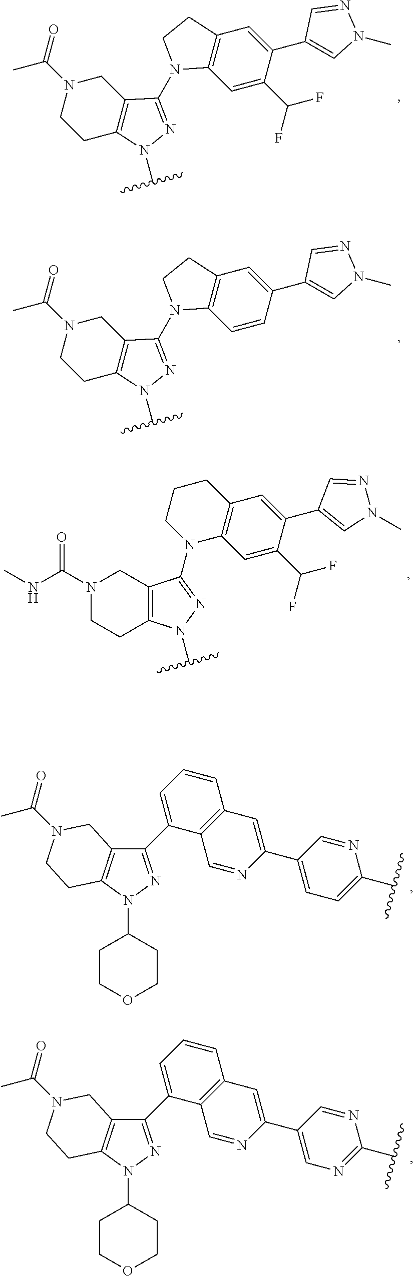

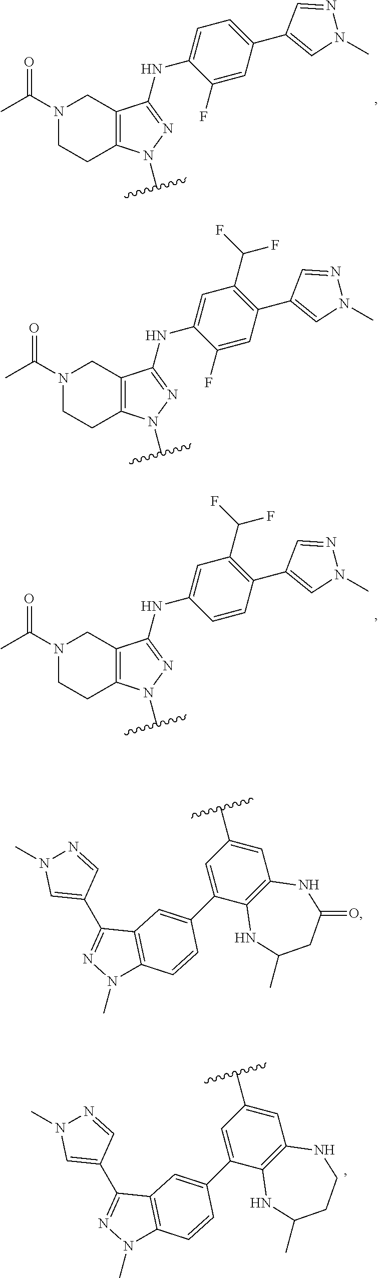

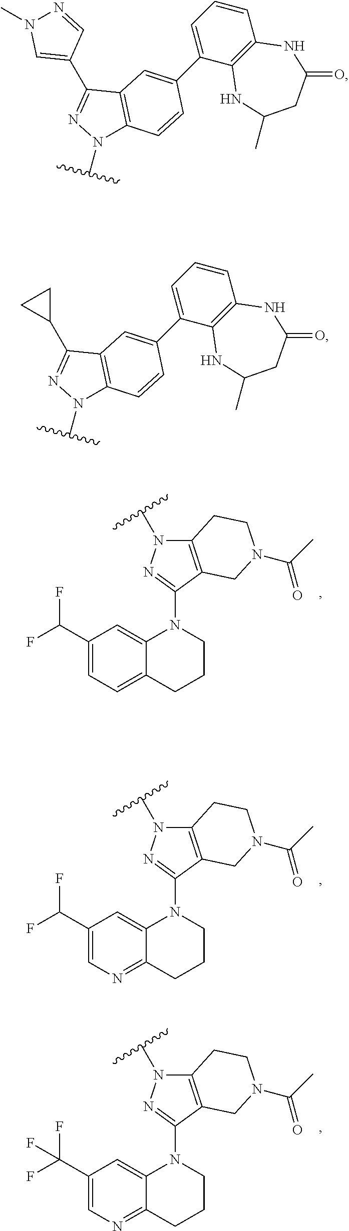 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2]

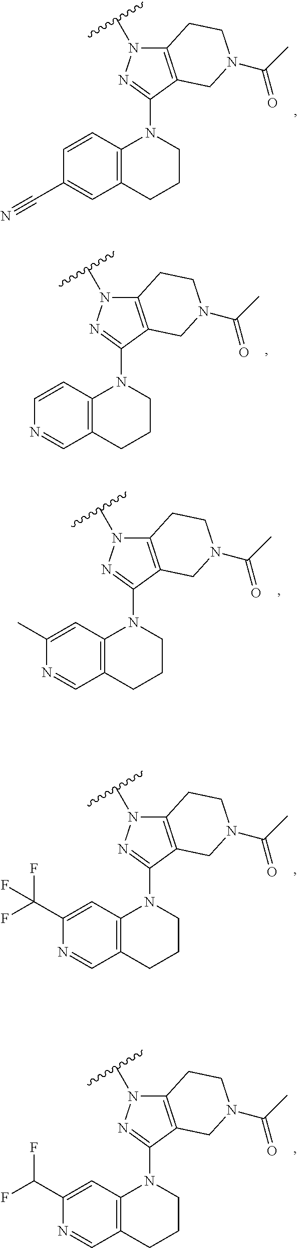

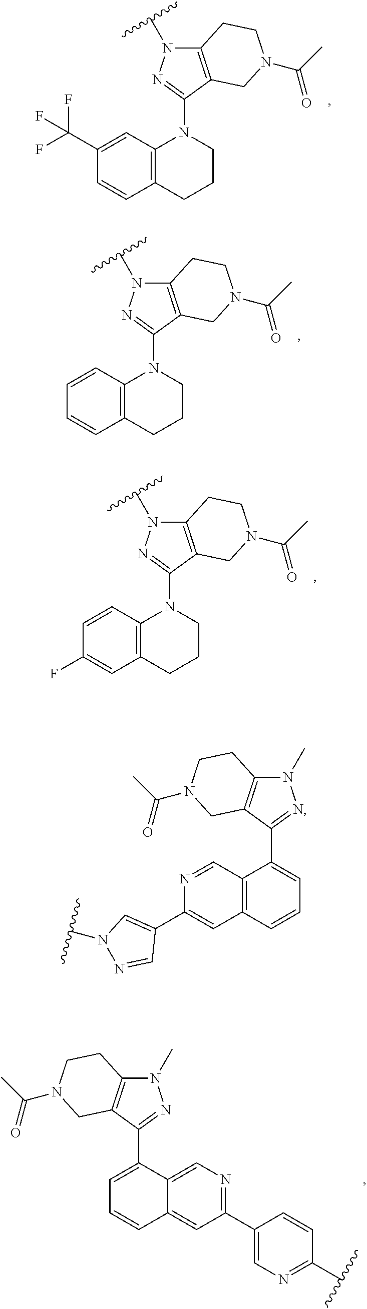

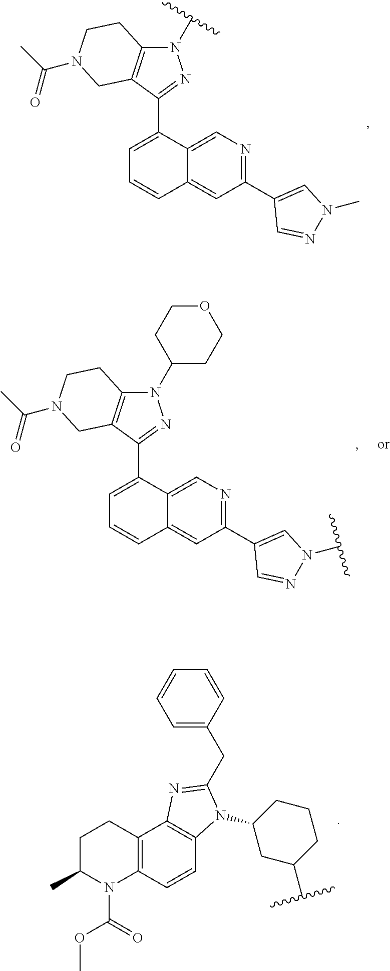

94. The compound of any one of claims 1-93, or a pharmaceutically acceptable salt or solvate thereof, wherein L is absent.

95. The compound of any one of claims 1-93, or a pharmaceutically acceptable salt or solvate thereof, wherein L comprises substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted 3- to 10-membered heterocycloalkyl, substituted or unsubstituted aryl, substituted or unsubstituted heteroaryl, or combinations thereof.

96. The compound of any one of claims 1-93, or a pharmaceutically acceptable salt or solvate thereof, wherein L is absent or 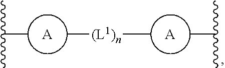
wherein:
each A is independently absent, substituted or unsubstituted monocyclic C₃-C₁₀cycloalkyl, substituted or unsubstituted bridged bicyclic C₃-C₁₀cycloalkyl, substituted or unsubstituted fused bicyclic C₃-C₁₀cycloalkyl, substituted or unsubstituted Spiro bicyclic C₃-C₁₀cycloalkyl, substituted or unsubstituted monocyclic 3- to 10-membered heterocycloalkyl, substituted or unsubstituted bridged bicyclic 3- to 10-membered heterocycloalkyl, substituted or unsubstituted fused bicyclic 3- to 10-membered heterocycloalkyl, substituted or unsubstituted spiro bicyclic 3- to 10-membered heterocycloalkyl, substituted or unsubstituted aryl, or substituted or unsubstituted heteroaryl, wherein each A is independently unsubstituted or substituted with x R^{2b};
each L¹ is independently absent, 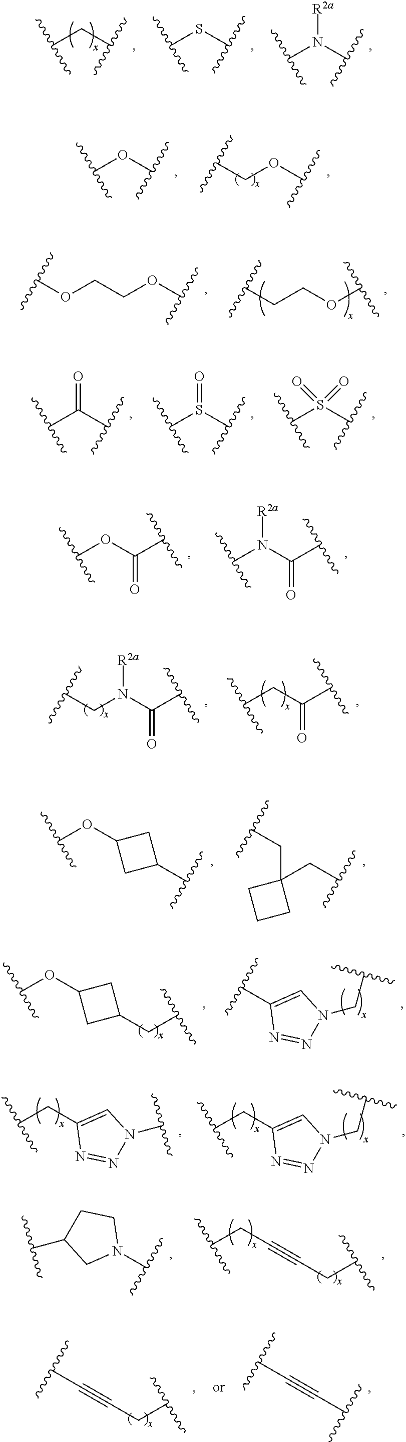
wherein each L¹ is independently unsubstituted or substituted with x R^{2b};
n is 1, 2, 3, 4, 5, or 6;
each x is independently 1, 2, 3, 4, 5, 6, 7, or 8;
each R^{2a} is independently hydrogen or substituted or unsubstituted C₁-C₆alkyl; and
each R^{2b} is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, —OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b});
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

97. The compound of claim 96, or a pharmaceutically acceptable salt or solvate thereof, wherein:
each A is independently absent, 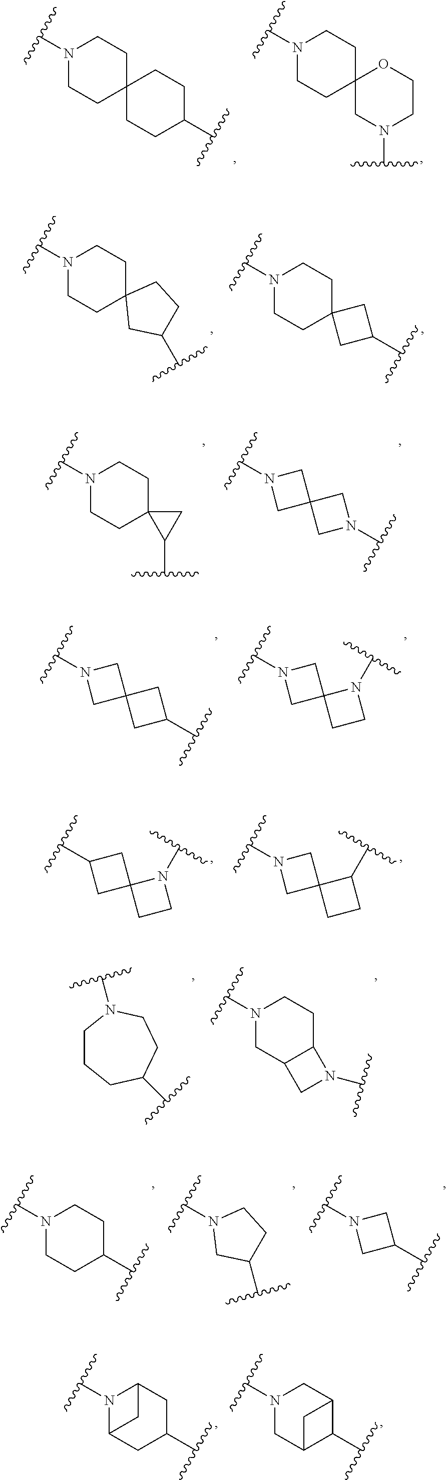 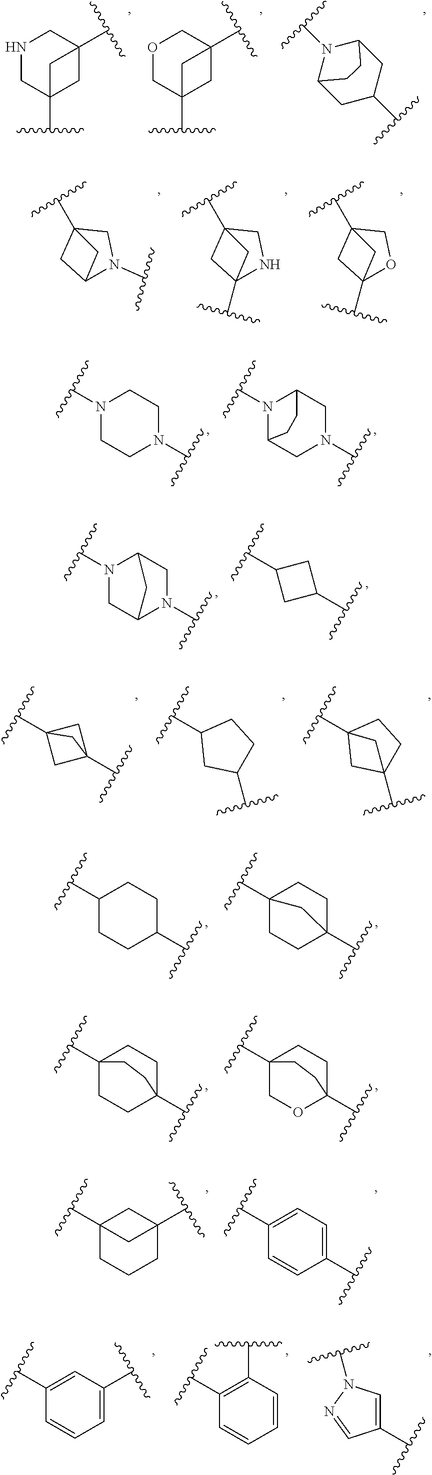 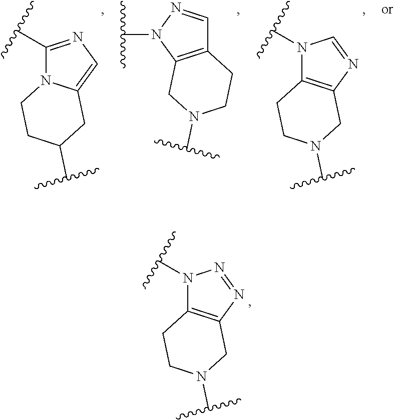
wherein each A is independently unsubstituted or substituted with x R^{2b};
(L¹)ₙ is absent,

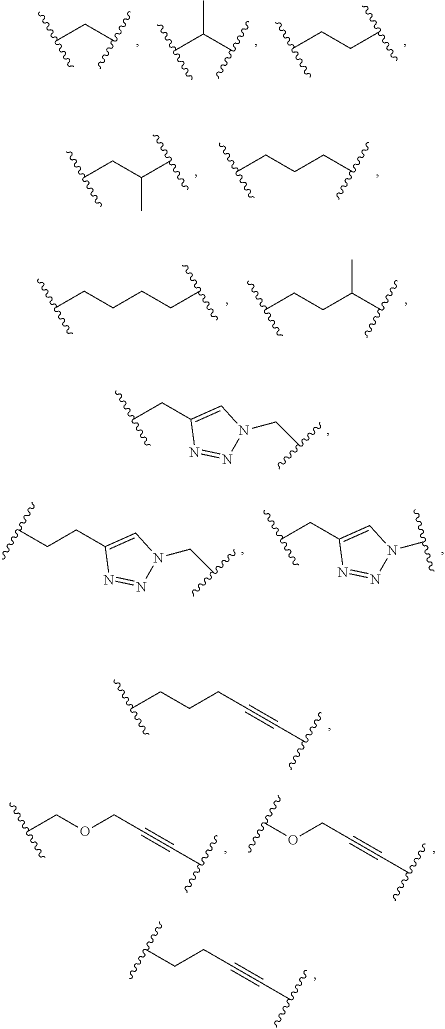

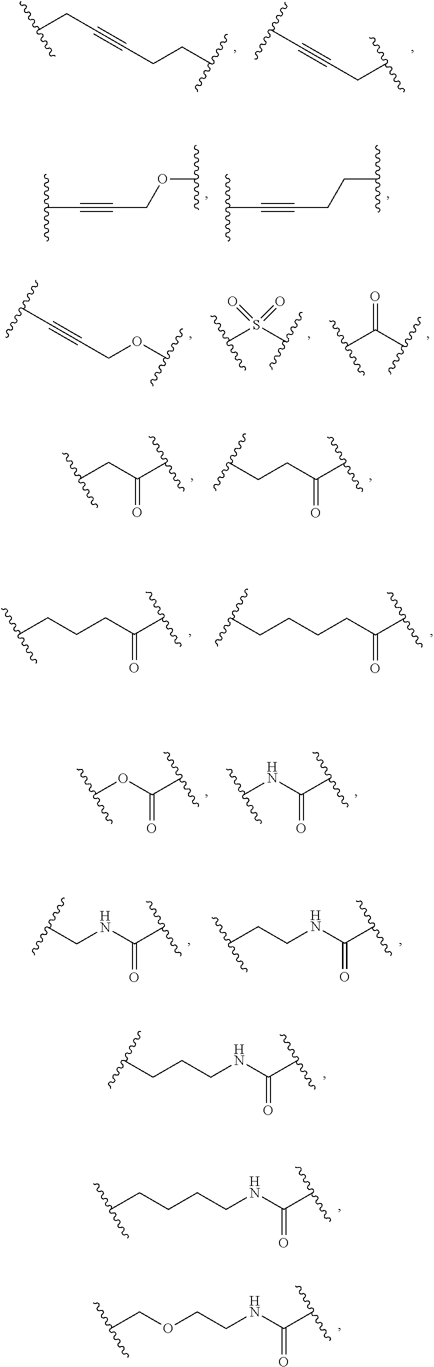

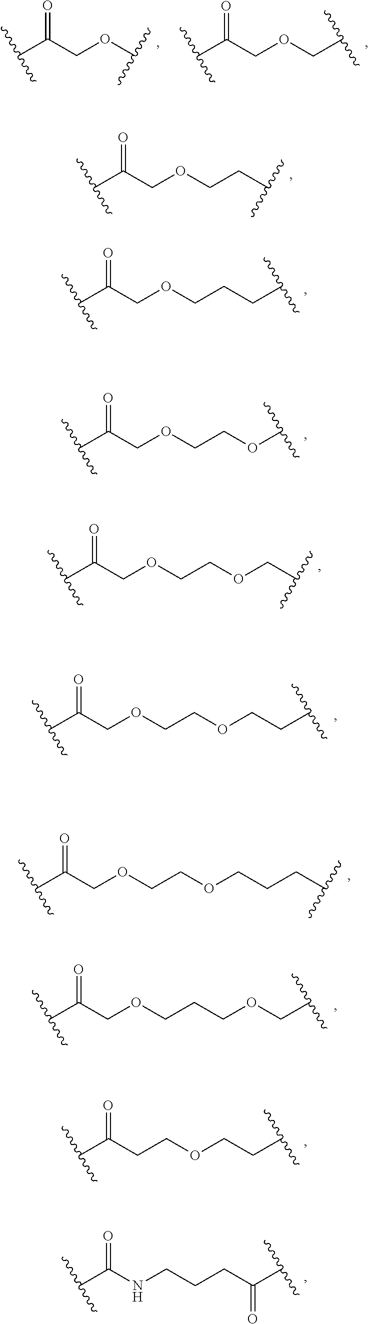

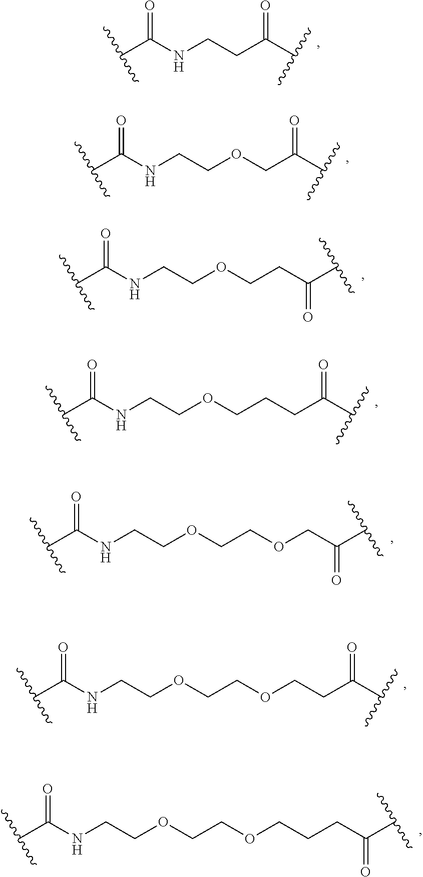

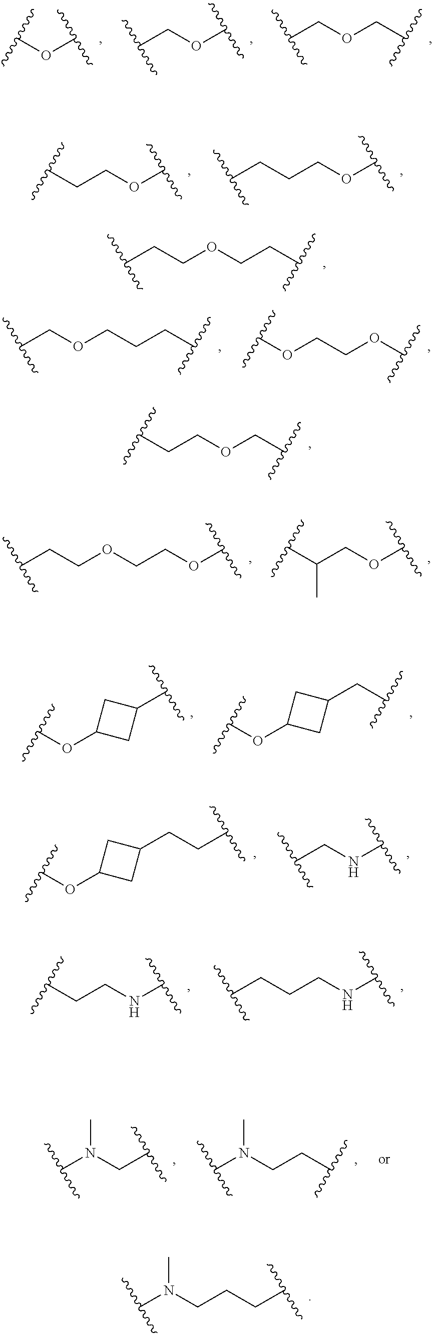

98. The compound of any one of claims 1-93, or a pharmaceutically acceptable salt or solvate thereof, wherein L is absent or 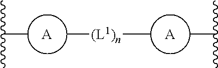
wherein:
each A is independently absent, 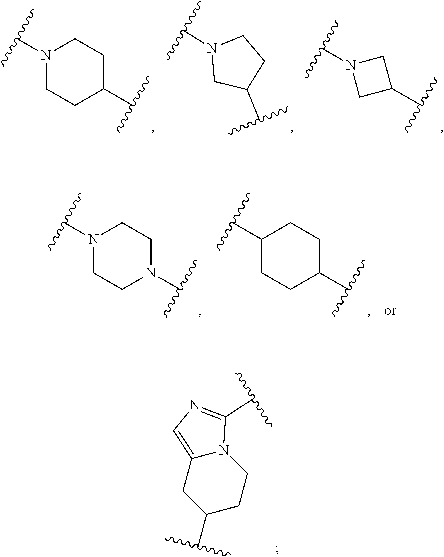 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]
each L¹ is independently absent, 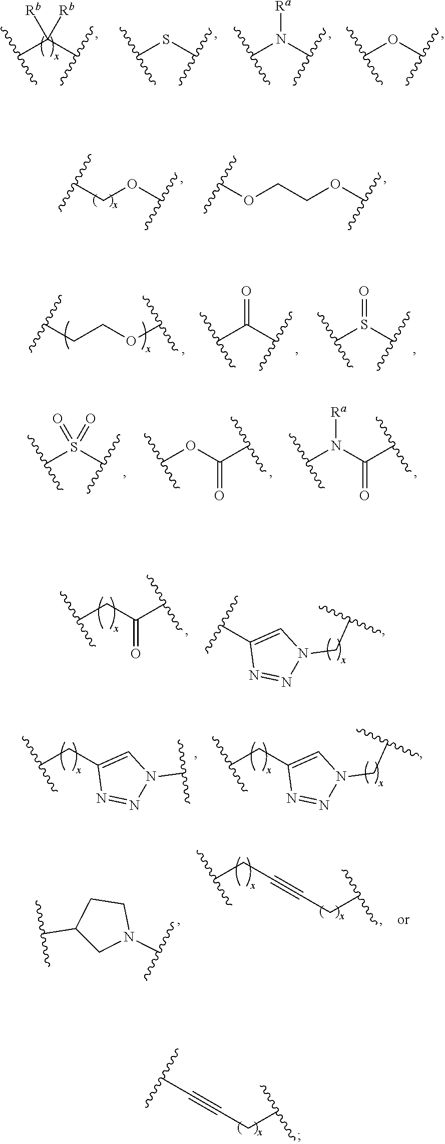
n is 1, 2, or 3; each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

99. The compound of any one of claims 96-98, or a pharmaceutically acceptable salt or solvate thereof, wherein:
(L¹)ₙ is absent,

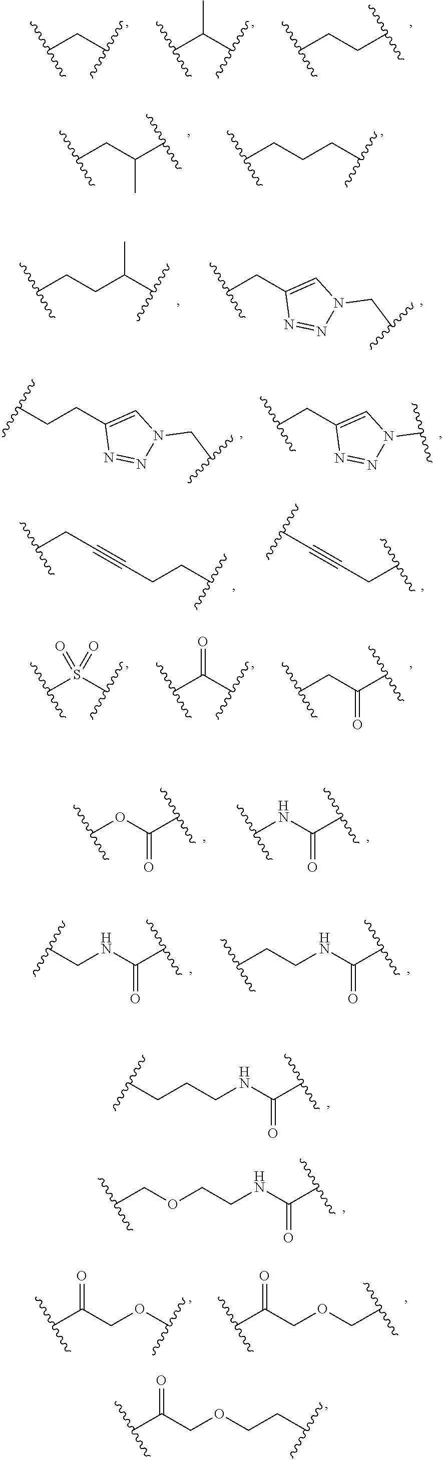

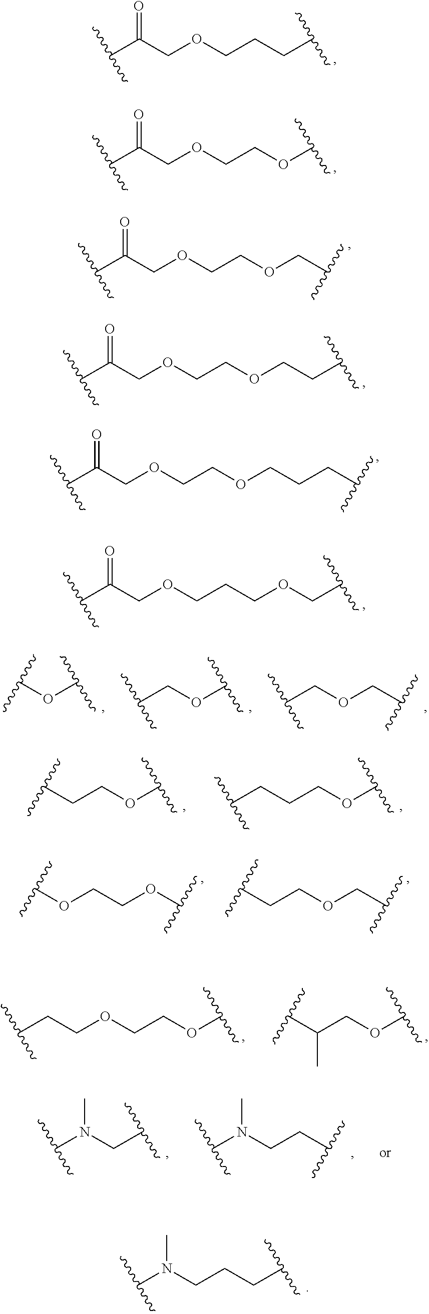

100. The compound of any one of claims 1-99, or a pharmaceutically acceptable salt or solvate thereof, wherein L has one of the following structures: 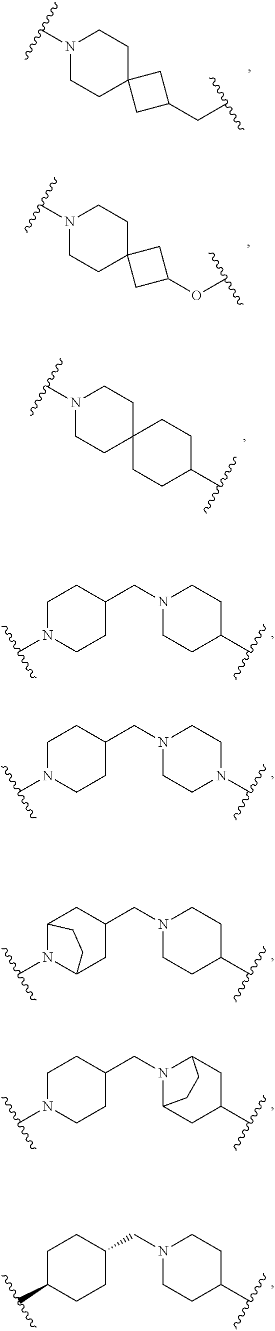 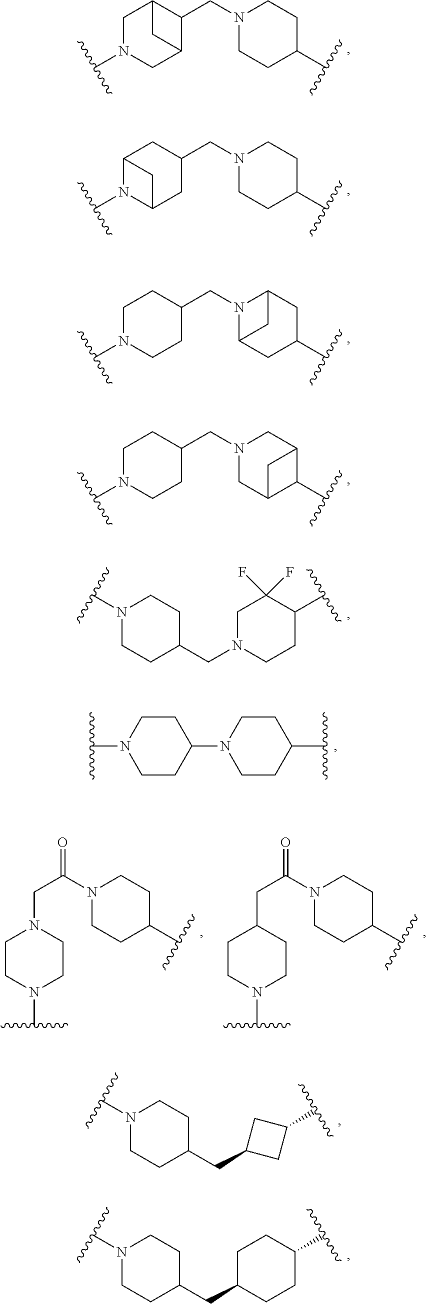 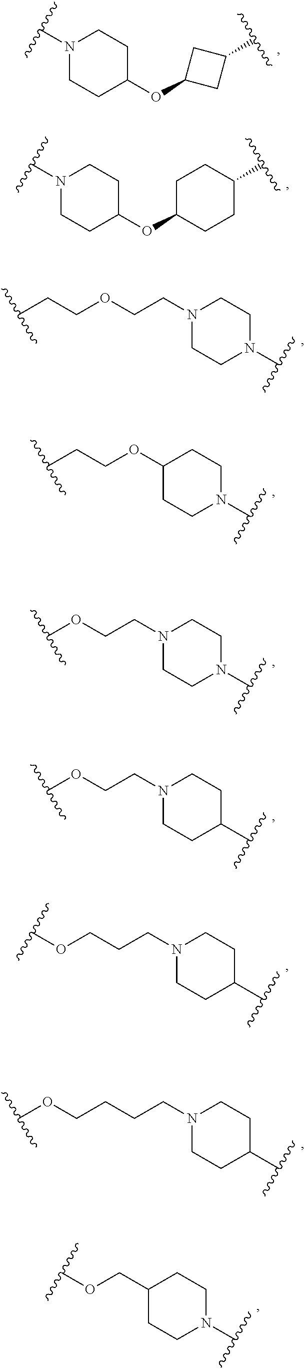 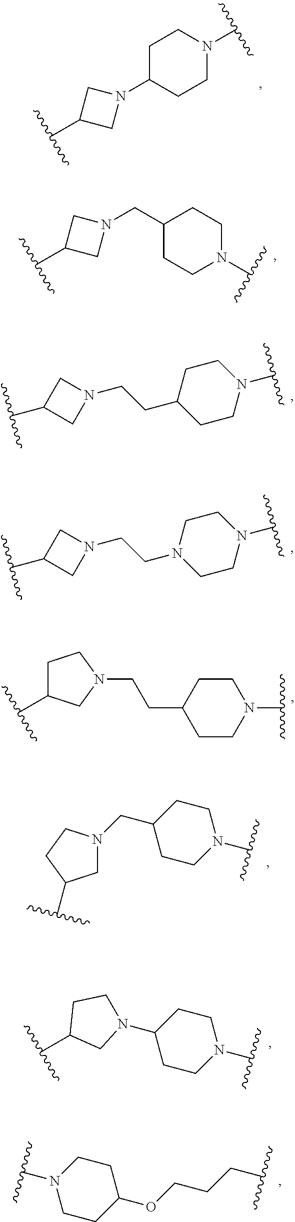 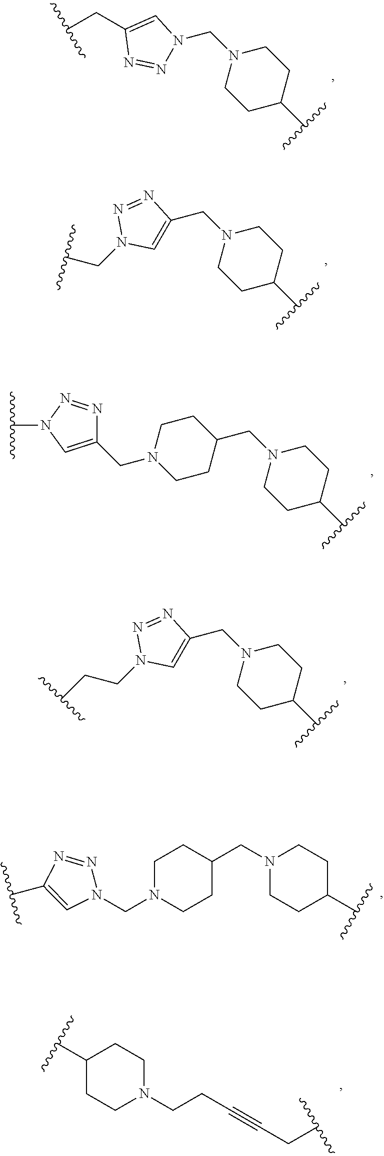 [SMILES: *Cc1cn(CN2CCC(*)CC2)nn1 ; *Cn1cc(CN2CCC(*)CC2)nn1 ; *C1CCN(CC2CCN(Cc3cn(*)nn3)CC2)CC1 ; *CCn1cc(CN2CCC(*)CC2)nn1 ; *c1cn(CN2CCC(CN3CCC(*)CC3)CC2)nn1 ; *CC#CCCN1CCC(*)CC1] 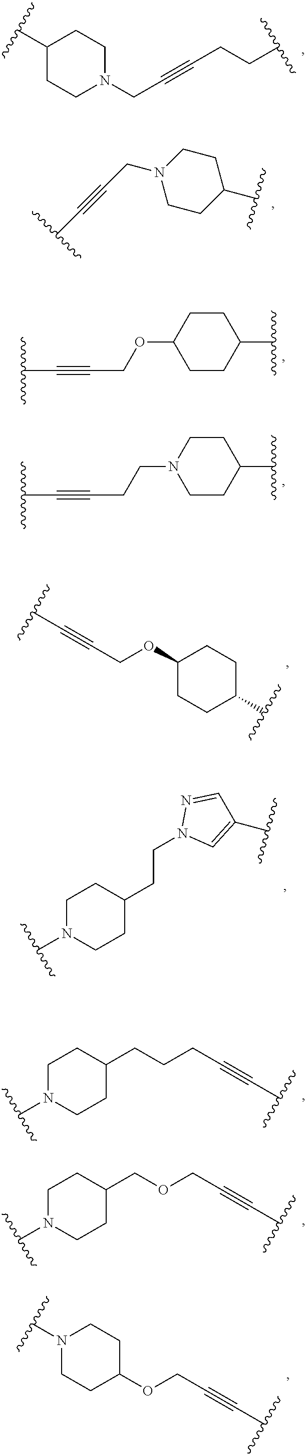 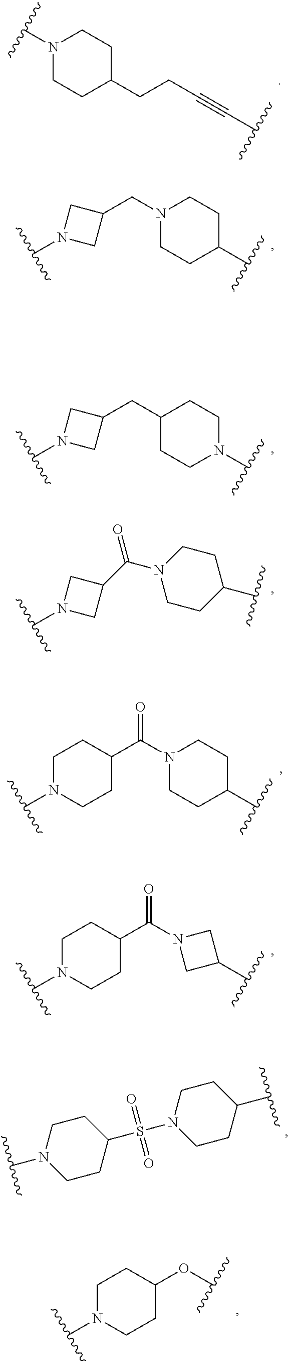 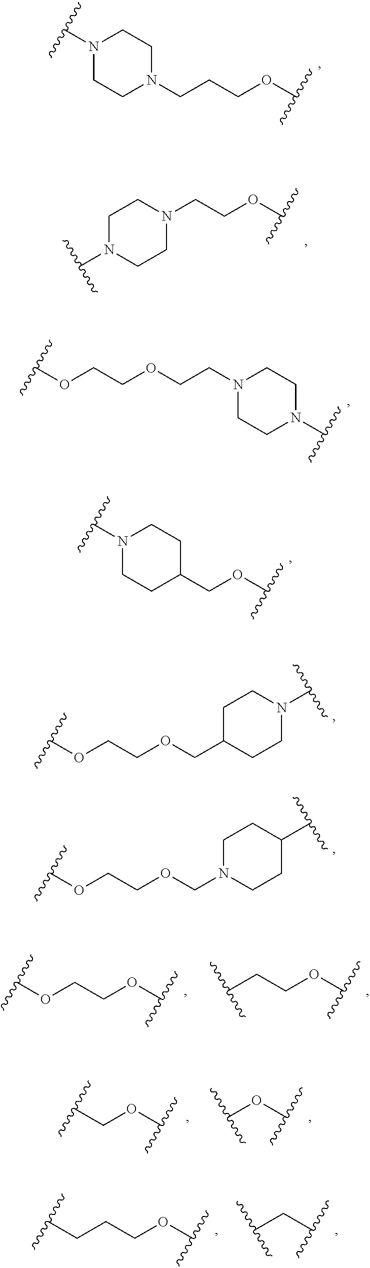 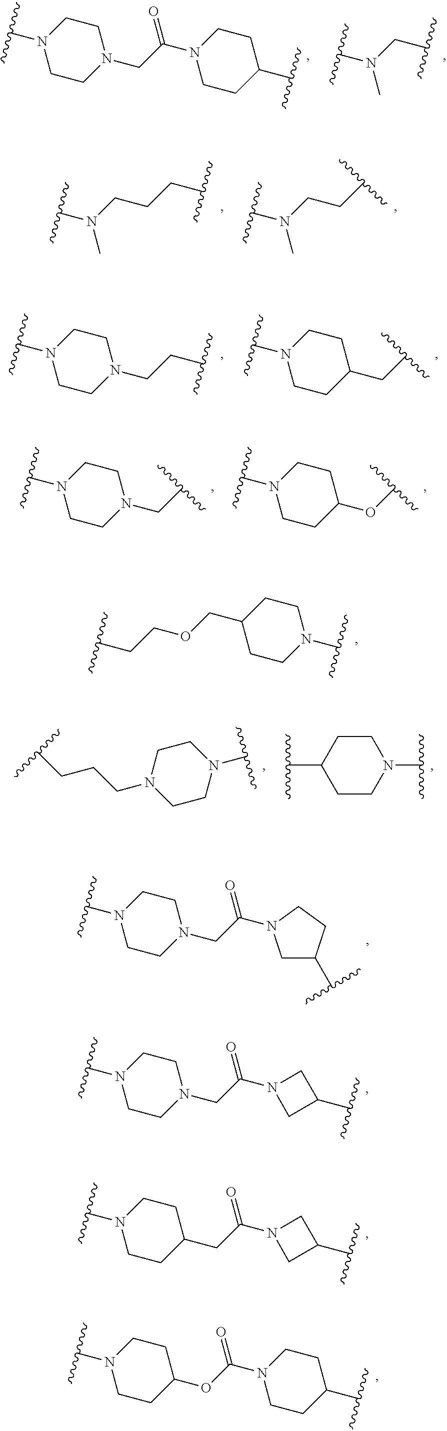 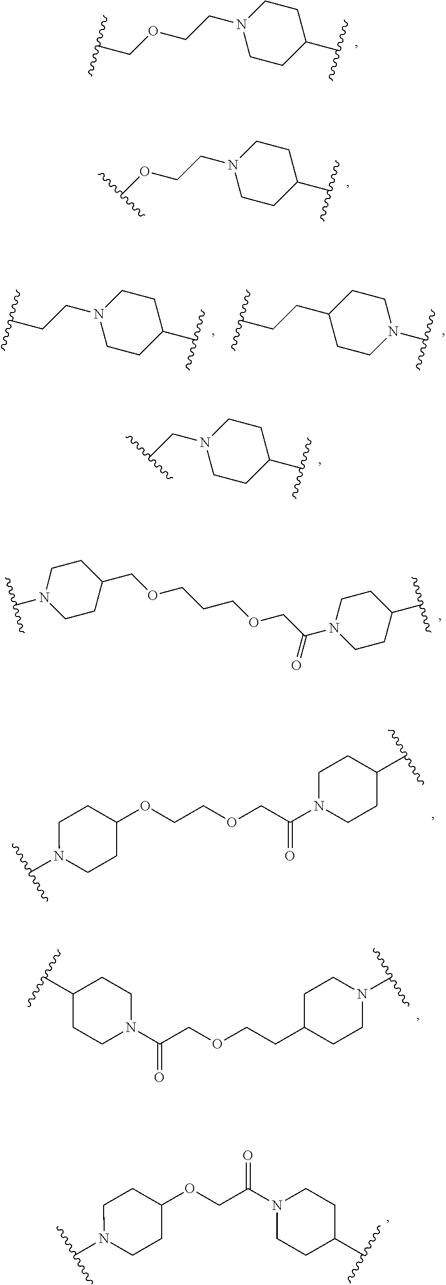 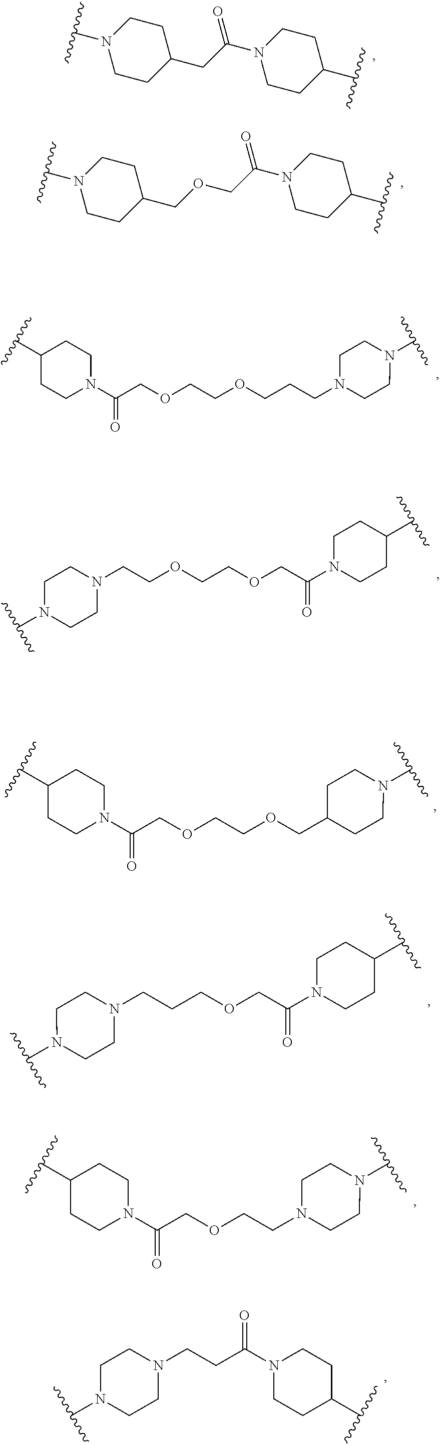 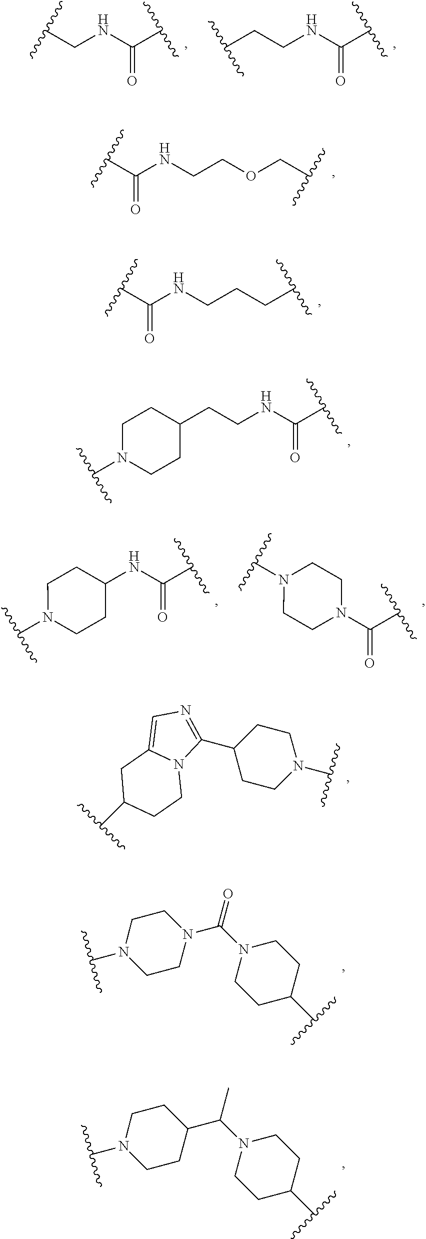 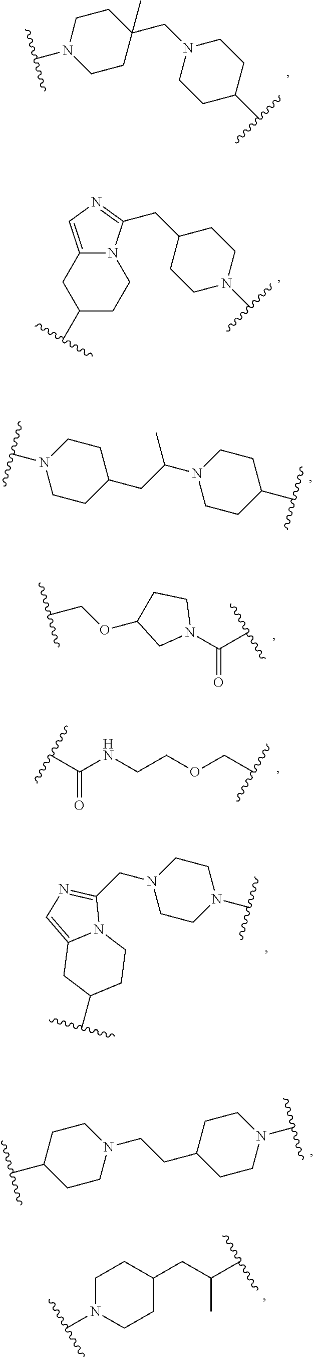 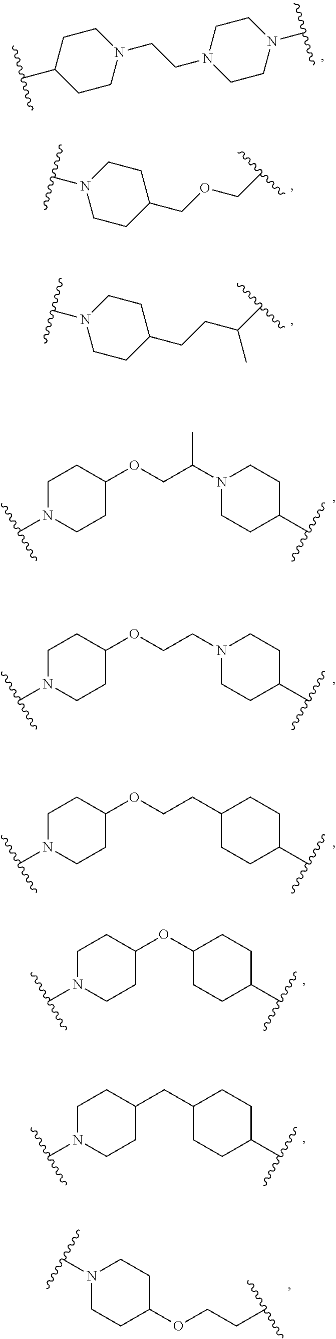 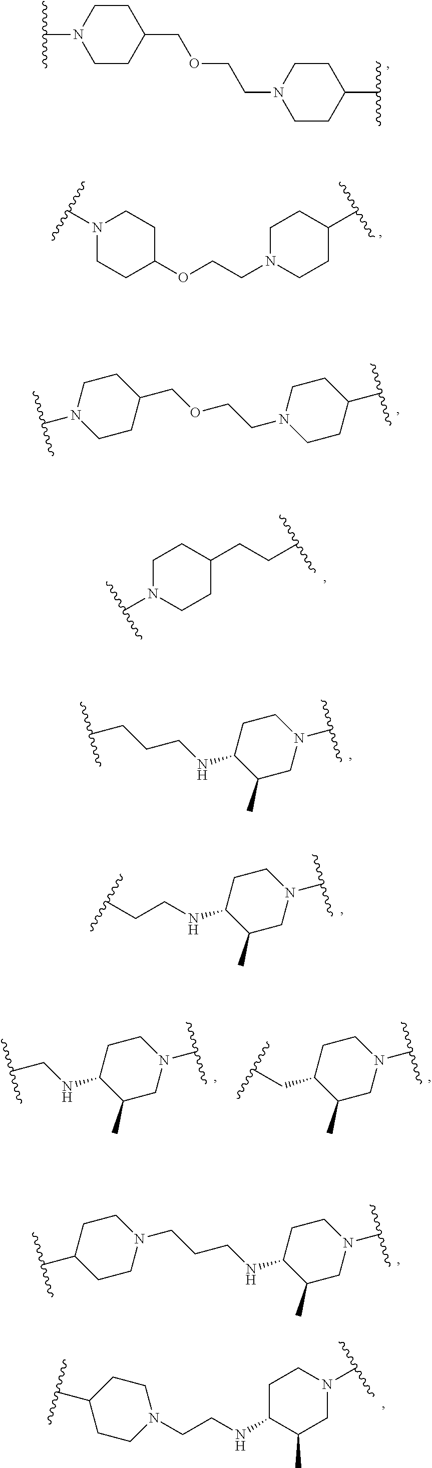 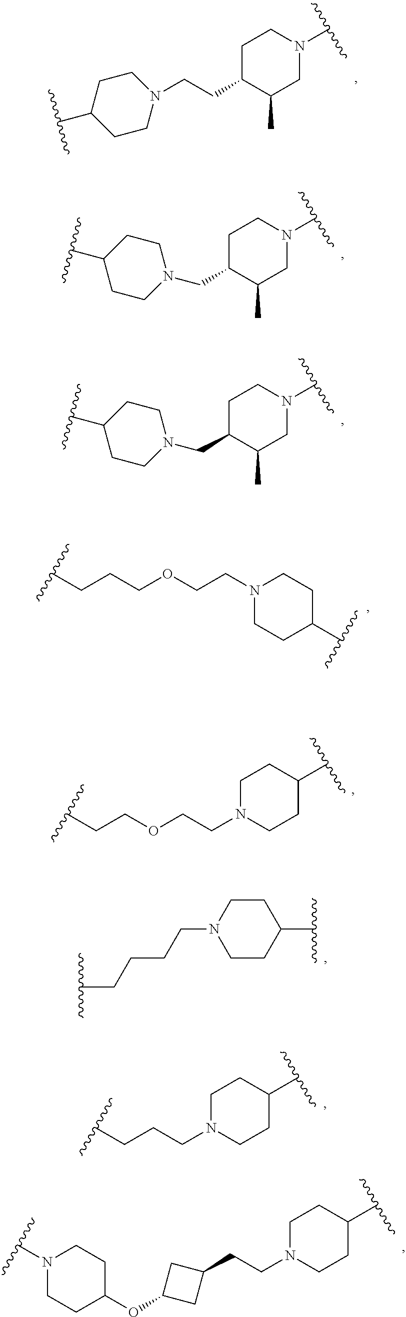 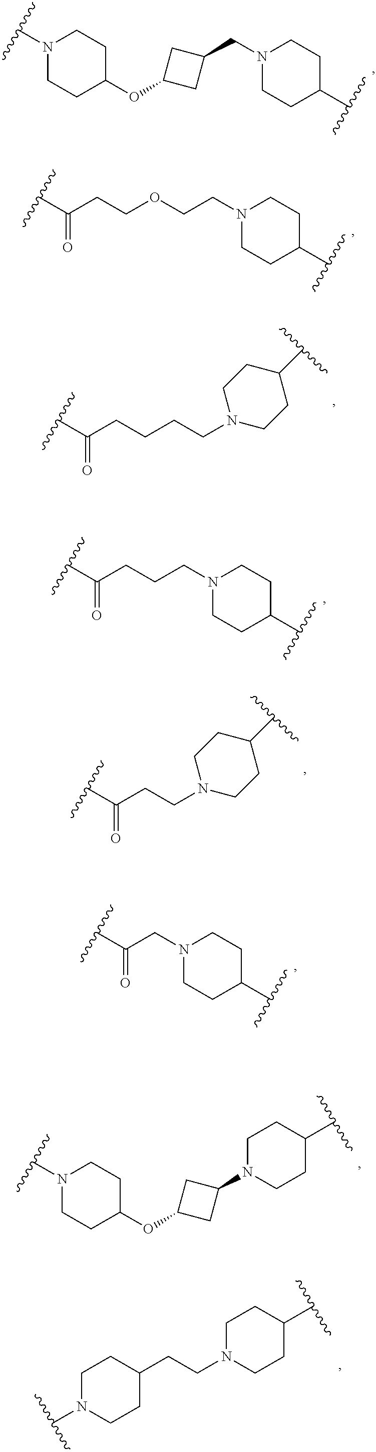 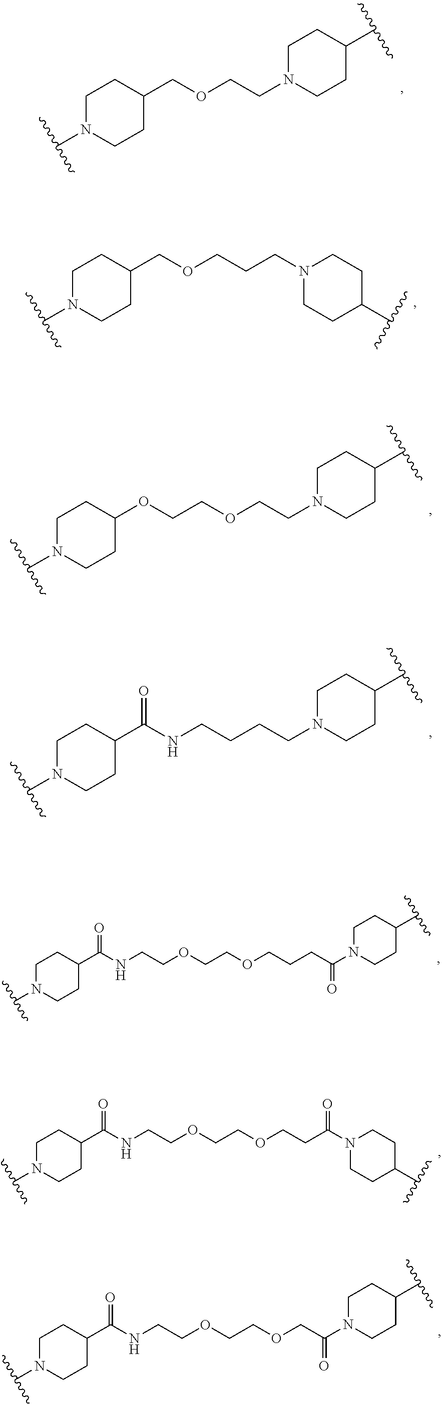 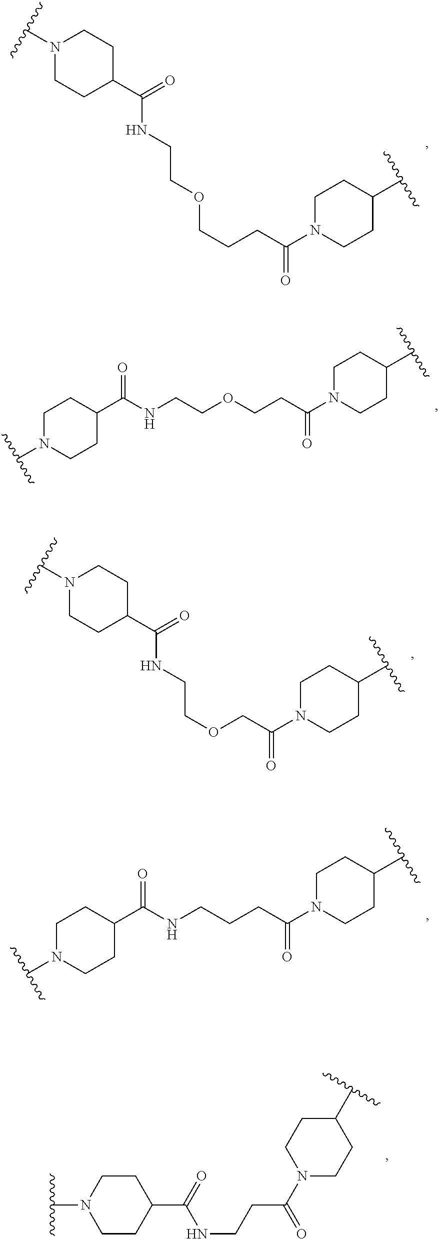 [SMILES: *C1CCN(C(=O)CCCOCCNC(=O)C2CCN(*)CC2)CC1 ; *C1CCN(C(=O)CCOCCNC(=O)C2CCN(*)CC2)CC1 ; *C1CCN(C(=O)COCCNC(=O)C2CCN(*)CC2)CC1 ; *C1CCN(C(=O)CCCNC(=O)C2CCN(*)CC2)CC1 ; *C1CCN(C(=O)CCNC(=O)C2CCN(*)CC2)CC1] 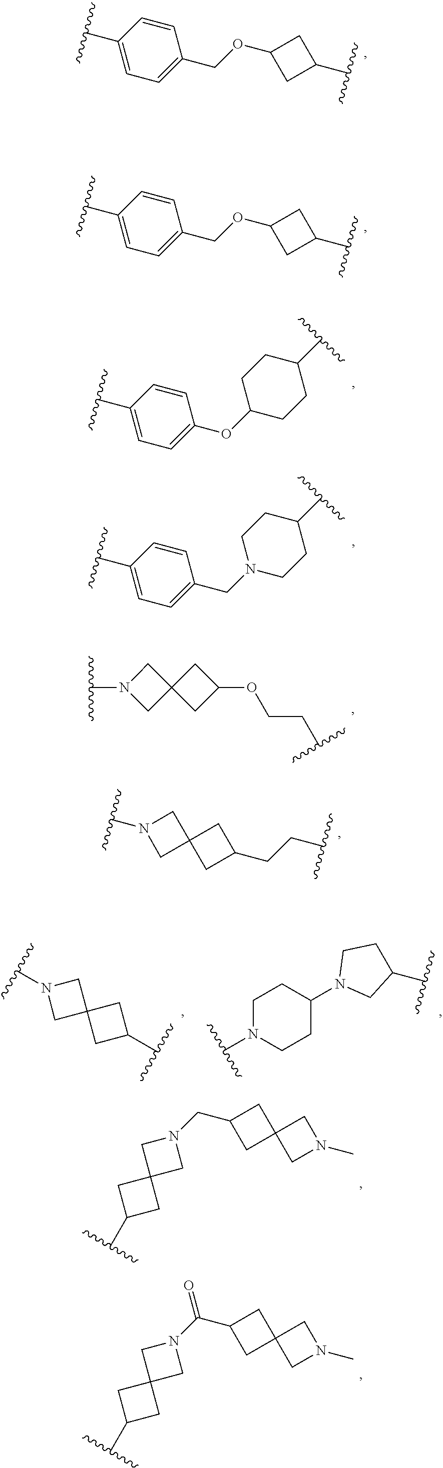 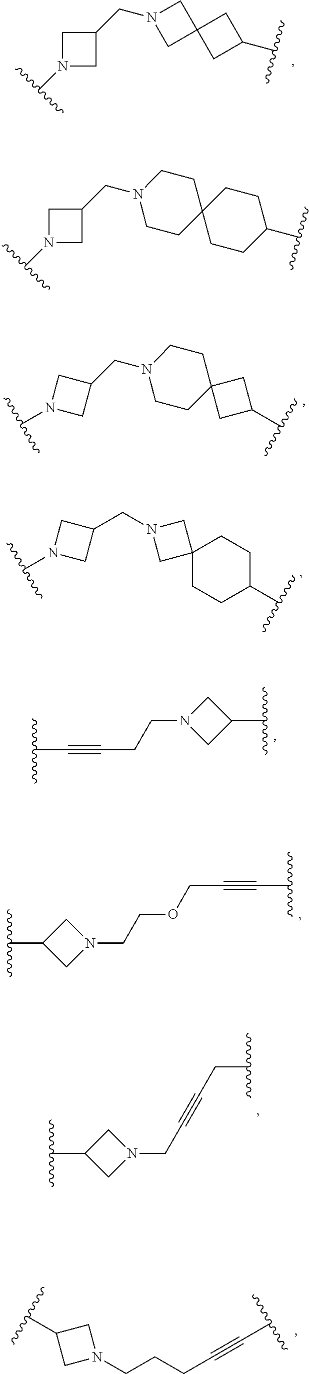 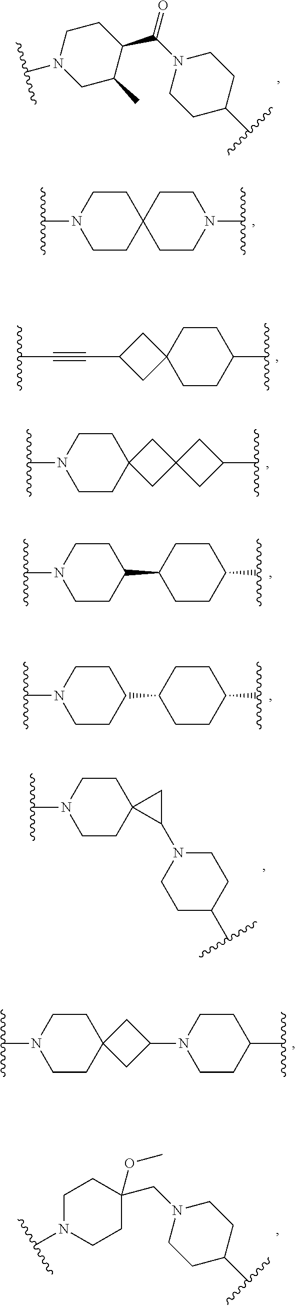 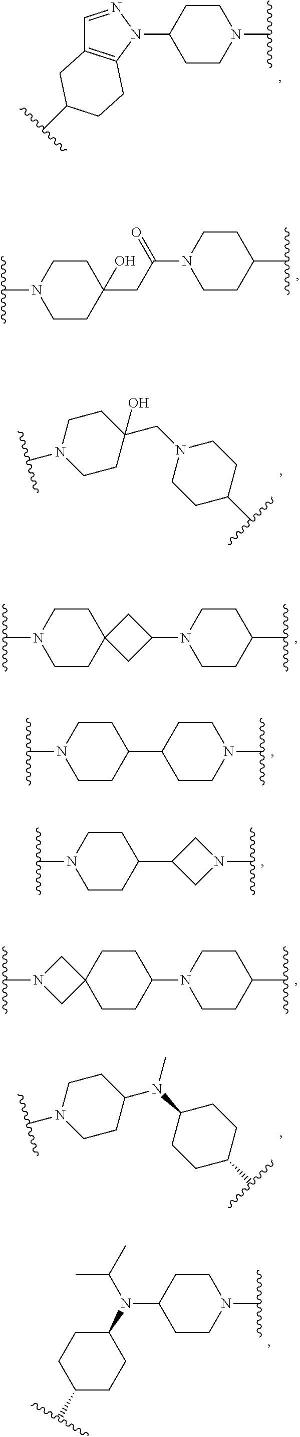 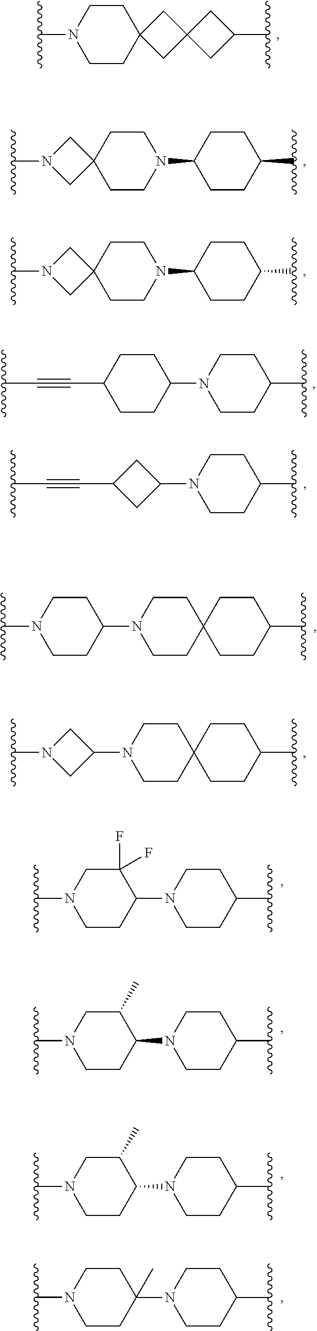 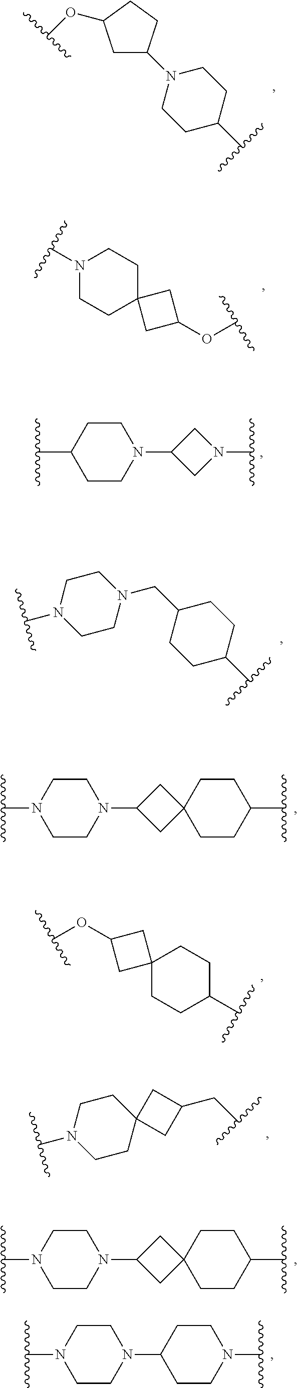 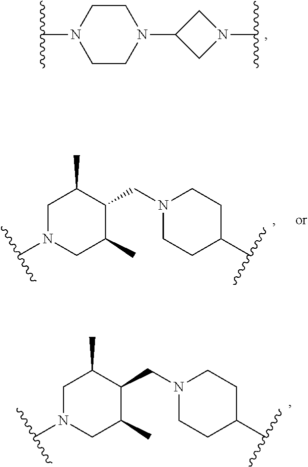 [SMILES: *N1CCN(C2CN(*)C2)CC1 ; *C1CCN(C[C@H]2[C@H](C)CN(*)C[C@@H]2C)CC1 ; *C1CCN(C[C@@H]2[C@H](C)CN(*)C[C@@H]2C)CC1]
or a pharmaceutically acceptable salt or solvate thereof.

101. The compound of any one of claims 1-99, or a pharmaceutically acceptable salt or solvate thereof, wherein L has one of the following structures: 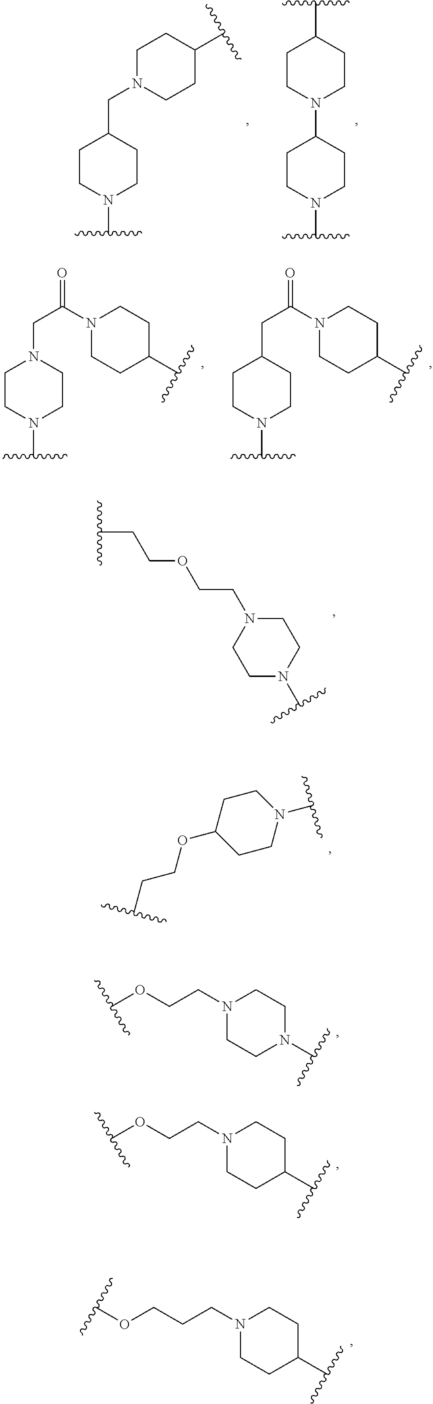 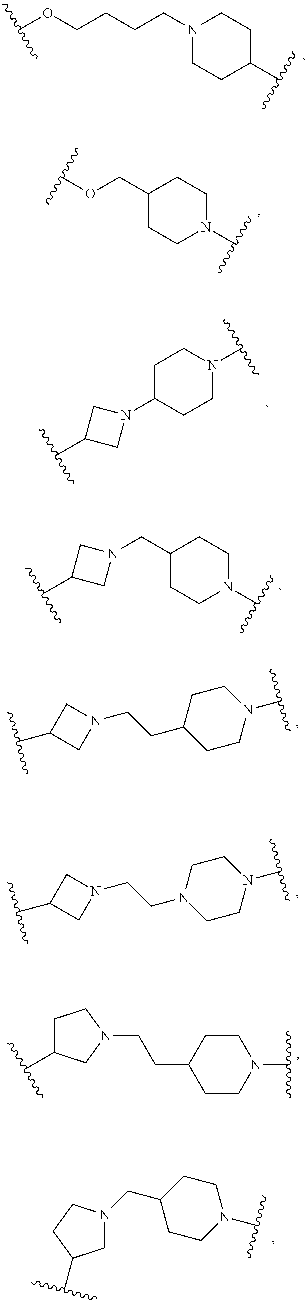 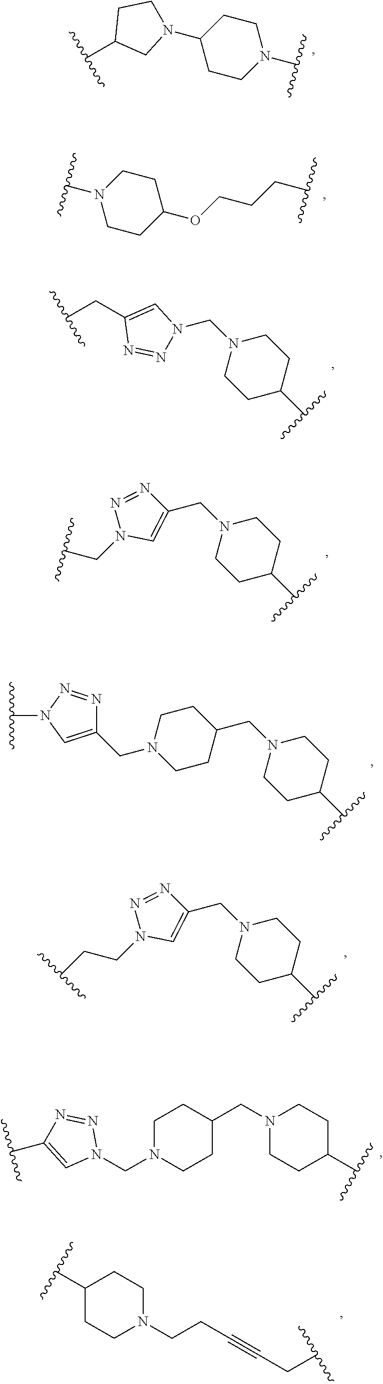 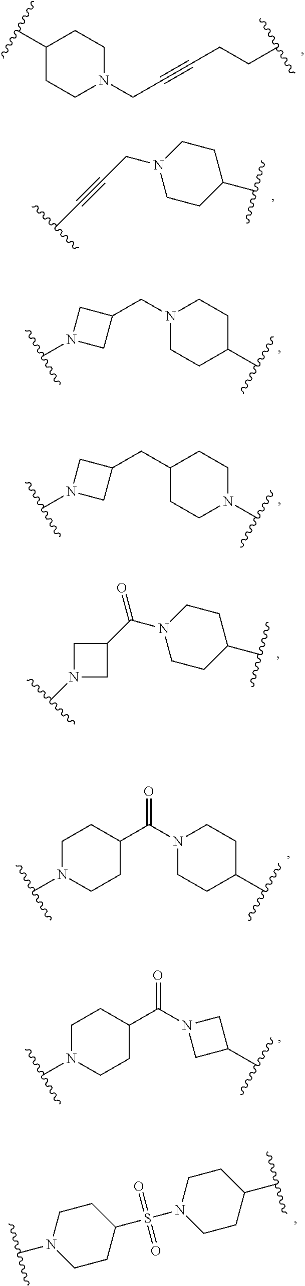 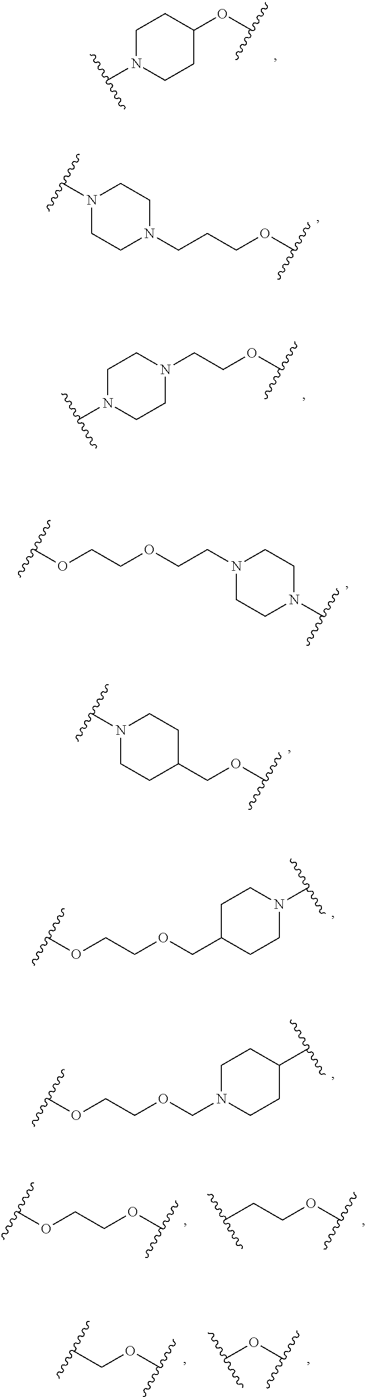 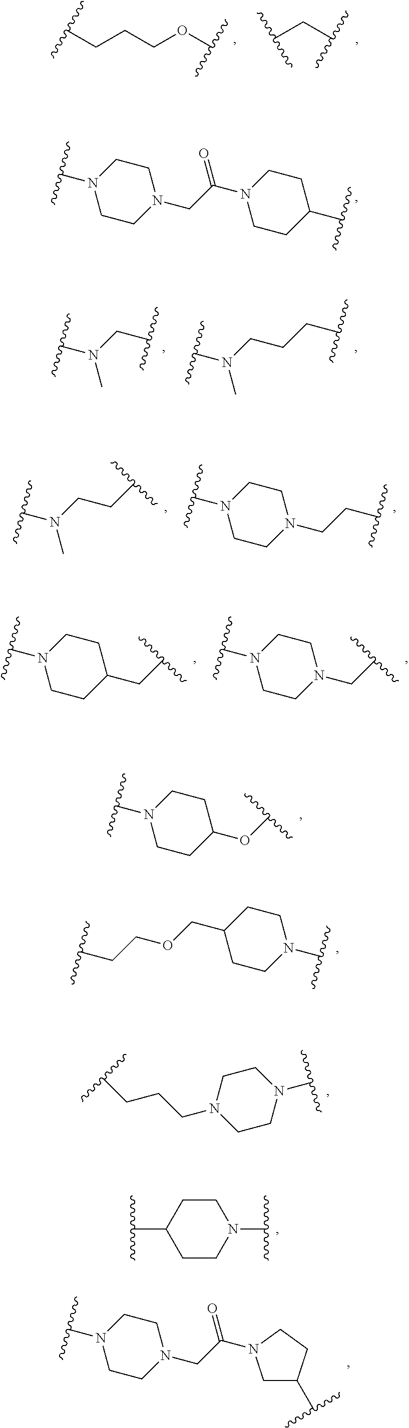 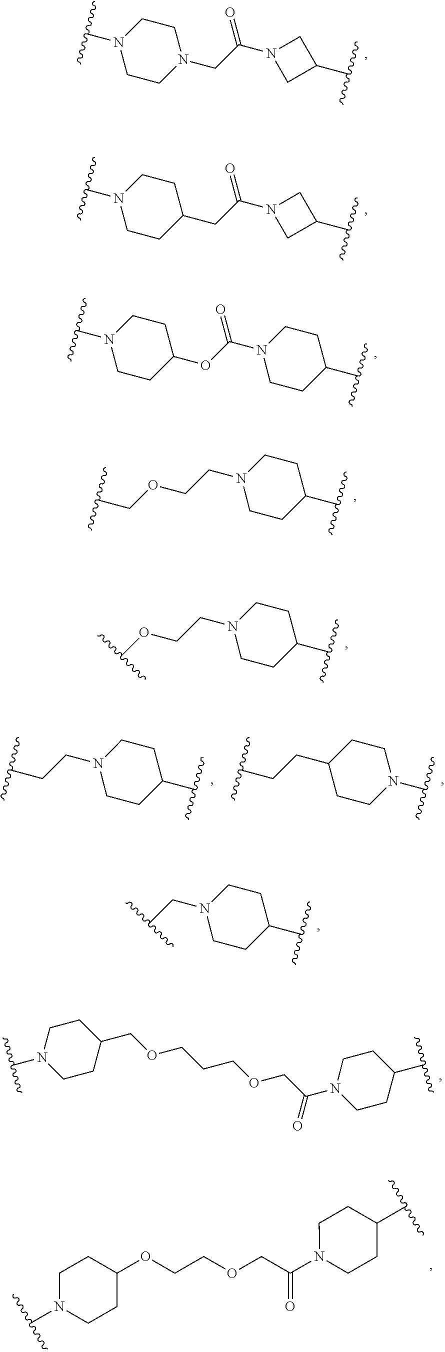 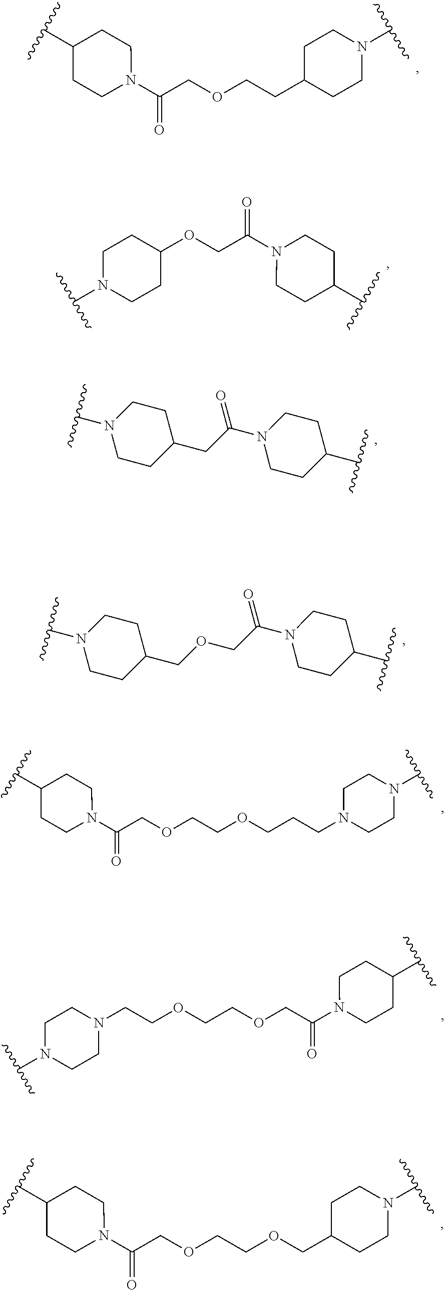 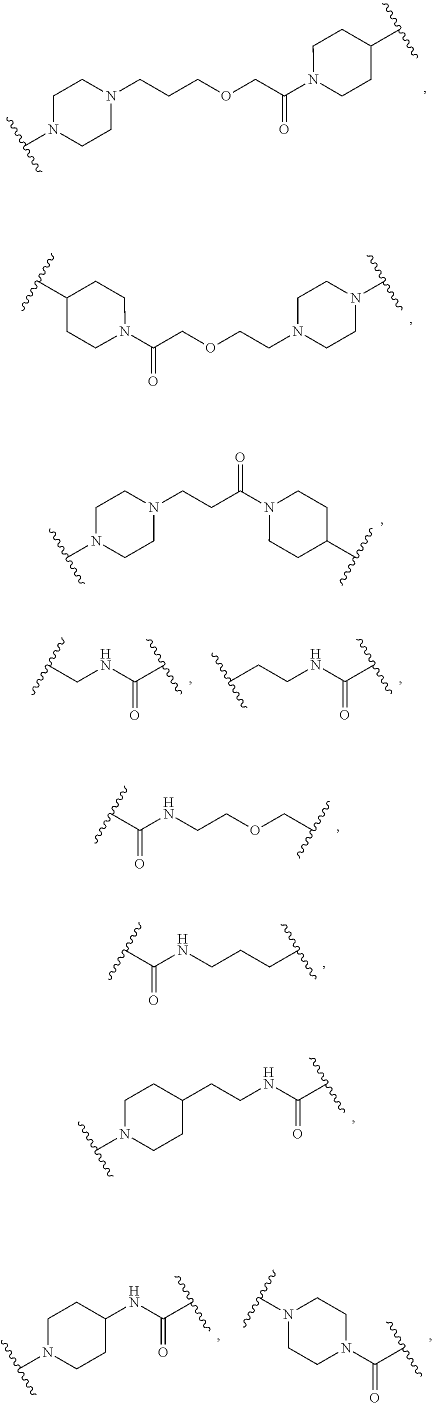 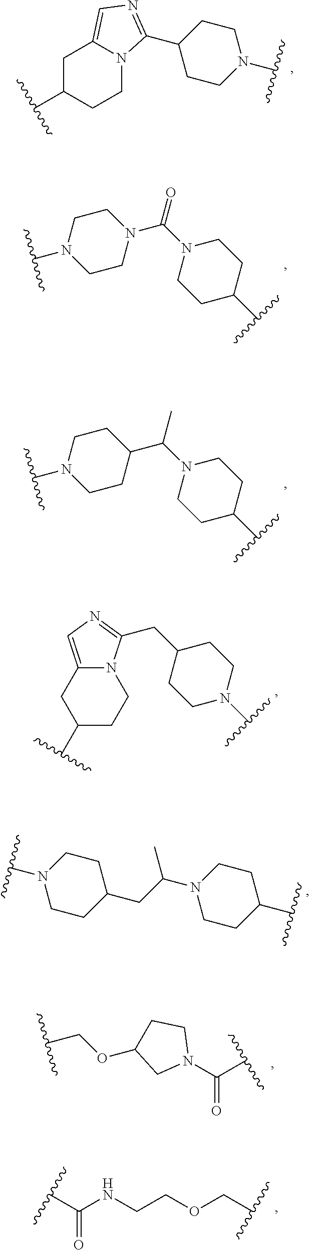 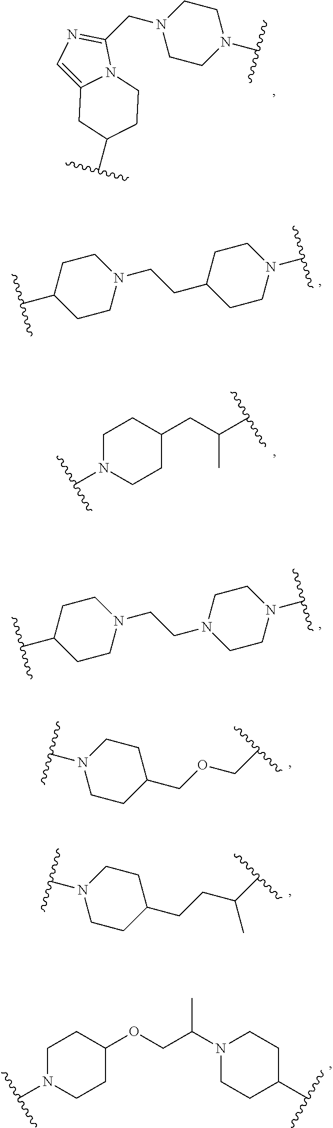 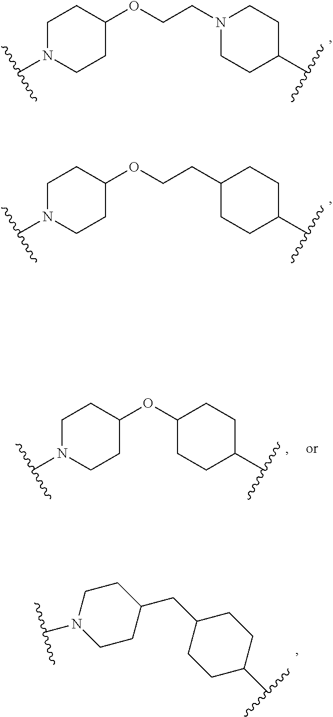 [SMILES: *C1CCN(CCOC2CCN(*)CC2)CC1 ; *C1CCC(CCOC2CCN(*)CC2)CC1 ; *C1CCC(OC2CCN(*)CC2)CC1 ; *C1CCC(CC2CCN(*)CC2)CC1]
or a pharmaceutically acceptable salt or solvate thereof.

102. A compound of Table 1, or a pharmaceutically acceptable salt or solvate thereof.

103. A stable ternary complex comprising:
a. one or more disease-dependent proteins (DDPs);
b. Androgen Receptor (AR); and
c. heterobifunctional conditional inhibitor compound of any one of claims 1-105;
wherein DDP and AR are present in a cell of interest (COI) and the relative abundance of the AR in the COI is greater than the relative abundance of the DDP in the COI.

104. The stable ternary complex of claim 103, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

105. The stable ternary complex of claim 103, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

106. The stable ternary complex of claim 103, wherein the DDP is CREB-binding protein (CBP)/p300.

107. A stable ternary complex comprising:
a. CBP/p300;
b. Androgen receptor (AR); and
c. heterobifunctional conditional inhibitor compound of any one of claims 1-105;
wherein CBP/p300 and DP are present in a cell of interest (COI) and the relative abundance of the DP in the COI is greater than the relative abundance of CBP/p300 in the COI.

108. A method of selectively inhibiting the activity of a disease-dependent protein (DDP) in a cell of interest (COI) of a mammal comprising administering a heterobifunctional compound of any one of claims 1-102, or a pharmaceutically acceptable salt or solvate thereof, wherein the COI expresses the androgen receptor (AR).

109. The method of claim 108, wherein the heterobifunctional compound of any one of claims 1-102, or a pharmaceutically acceptable salt or solvate thereof, inhibits the activity of the DDP in the COI but does not inhibit the activity of the DDP in cells expressing the DDP and not expressing the AR.

110. The method of claim 108, wherein the AR is overexpressed, overactive or both overexpressed and overactive in the COI.

111. The method of any one of claims 108-110, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRAT5), splicing factor 3b subunit 1 (SF3B1), WEE1, 20S proteasome subunits, Steroid Receptor Coactivator 1 (SRC1), SRC2, or SRC3.

112. The method of any one of claims 108-111, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

113. The method of any one of claims 108-111, wherein the DDP is CREB-binding protein (CBP)/p300.

114. A method of treating cancer in a mammal comprising administering to the mammal a heterobifunctional compound of any one of claims 1-102.

115. A method of treating cancer in a mammal comprising administering to the mammal a therapeutically effective amount of a compound of any one of claims 1-102, or a pharmaceutically acceptable salt or solvate thereof.

116. The method of claim 115, wherein the cancer is a hormone dependent cancer.

117. The method of claim 115, wherein the cancer is prostate cancer.

118. A method of treating an androgen receptor dependent or androgen receptor mediated disease or condition in mammal comprising administering to the mammal a therapeutically effective amount of a compound according to any one of claims 1-102, or a pharmaceutically acceptable salt or solvate thereof.

119. The method of claim 118, wherein androgen receptor dependent or androgen receptor mediated disease or condition is selected from benign prostate hyperplasia, hirsutism, adenomas and neoplasms of the prostate, benign or malignant tumor cells containing the androgen receptor, prostate cancer, breast cancer, endometrial cancer, and uterine cancer.

120. A pharmaceutical composition comprising a compound of any one of claims 1-102, or a pharmaceutically acceptable salt or solvate thereof, and at least one pharmaceutically acceptable excipient.
